(19) United States
(12) Patent Application Publication
(10) Pub. No.: US 2014/0329814 A2
(43) Pub. Date: Nov. 6, 2014

(54) Compositions for Treatment of Cystic Fibrosis and Other Chronic Diseases

(71) Applicant: Vertex Pharmaceuticals Incorporated, Boston, MA (US)

(72) Inventors: Fredrick F. Van Goor, San Diego, CA (US); William Lawrence Burton, San Diego, CA (US)

(21) Appl. No.: 14/107,700

(22) Filed: Dec. 16, 2013

Related U.S. Application Data
(63) Continuation of application No. 12/910,727, filed on Oct. 22, 2010, now abandoned.

(51) Int. Cl. A61K 31/5377; A61K 31/357; A61K 31/443; A61K 31/506; A61K 31/404; A61K 31/4439; A61K 31/497; A61K 31/4709; A61K 31/4025

(52) U.S. Cl. CPC A61K 31/5377; A61K 31/4709; A61K 31/357; A61K 31/443; A61K 31/4025; A61K 31/404; A61K 31/4439; A61K 31/497; A61K 31/506
USPC 514/235.8; 514/255.05

(57) ABSTRACT

The present invention relates to pharmaceutical compositions comprising an inhibitor of epithelial sodium channel activity in combination with at least one ABC Transporter modulator compound of Formula A, Formula B, Formula C, or Formula D. The invention also relates to pharmaceutical formulations thereof, and to methods of using such compositions in the treatment of CFTR mediated diseases, particularly cystic fibrosis using the pharmaceutical combination compositions.

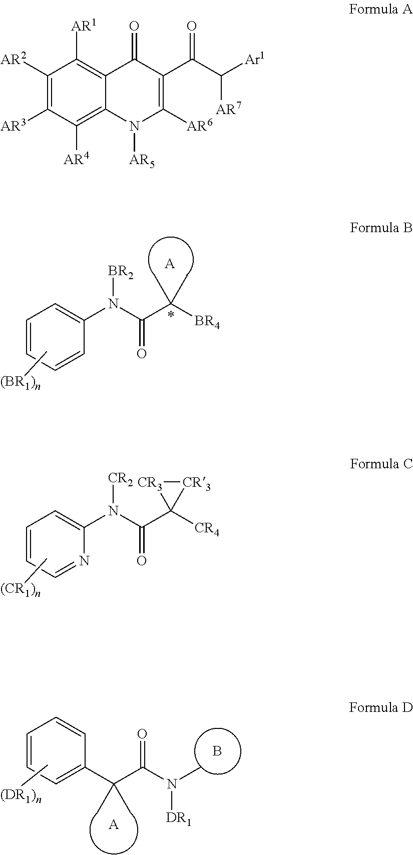

### CROSS REFERENCE TO RELATED APPLICATIONS

This application claims the benefit of U.S. Provisional Patent Application No. 61/254,180-filed on Oct. 22, 2009. The disclosure of the above referenced application is incorporated herein by reference in its entirety.

### TECHNICAL FIELD

The present invention relates to compositions for the treatment of cystic fibrosis (CF) and other chronic diseases, methods for preparing the compositions and methods for using the compositions for the treatment of CF and other chronic diseases, including chronic diseases involving regulation of fluid volumes across epithelial membranes.

### BACKGROUND

Cystic fibrosis (CF) is a recessive genetic disease that affects approximately 30,000 children and adults in the United States and approximately 30,000 children and adults in Europe. Despite progress in the treatment of CF, there is no cure.

CF is caused by mutations in the cystic fibrosis transmembrane conductance regulator (CFTR) gene that encodes an epithelial chloride ion channel responsible for aiding in the regulation of salt and water absorption and secretion in various tissues. Small molecule drugs, known as potentiators that increase the probability of CFTR channel opening, represent one potential therapeutic strategy to treat CF. Potentiators of this type are disclosed in WO 2006/002421, which is herein incorporated by reference in its entirety. Another potential therapeutic strategy involves small molecule drugs known as CF correctors that increase the number and function of CFTR channels. Correctors of this type are disclosed in WO 2005/075435, which are herein incorporated by reference in their entirety.

Specifically, CFTR is a cAMP/ATP-mediated anion channel that is expressed in a variety of cells types, including absorptive and secretory epithelia cells, where it regulates anion flux across the membrane, as well as the activity of other ion channels and proteins. In epithelia cells, normal functioning of CFTR is critical for the maintenance of electrolyte transport throughout the body, including respiratory and digestive tissue. CFTR is composed of approximately 1480 amino acids that encode a protein made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). A defect in this gene causes mutations in CFTR resulting in cystic fibrosis (“CF”), the most common fatal genetic disease in humans. Cystic fibrosis affects approximately one in every 2,500 infants in the United States. Within the general United States population, up to 10 million people carry a single copy of the defective gene without apparent ill effects. In contrast, individuals with two copies of the CF associated gene suffer from the debilitating and fatal effects of CF, including chronic lung disease.

In patients with CF, mutations in CFTR endogenously expressed in respiratory epithelia leads to reduced apical anion secretion causing an imbalance in ion and fluid transport. The resulting decrease in anion transport contributes to enhanced mucus accumulation in the lung and the accompanying microbial infections that ultimately cause death in CF patients. In addition to respiratory disease, CF patients typically suffer from gastrointestinal problems and pancreatic insufficiency that, if left untreated, results in death. In addition, the majority of males with cystic fibrosis are infertile and fertility is decreased among females with cystic fibrosis. In contrast to the severe effects of two copies of the CF associated gene, individuals with a single copy of the CF associated gene exhibit increased resistance to cholera and to dehydration resulting from diarrhea—perhaps explaining the relatively high frequency of the CF gene within the population.

Sequence analysis of the CFTR gene of CF chromosomes has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 1000 disease causing mutations in the CF gene have been identified (http://www.genet.sickkids.on.ca/cftr/app). The most prevalent mutation is a deletion of phenylalanine at position 508 of the CFTR amino acid sequence, and is commonly referred to as ΔF508-CFTR. This mutation occurs in approximately 70% of the cases of cystic fibrosis and is associated with a severe disease.

The deletion of residue 508 in ΔF508-CFTR prevents the nascent protein from folding correctly. This results in the inability of the mutant protein to exit the ER, and traffic to the plasma membrane. As a result, the number of channels present in the membrane is far less than observed in cells expressing wild-type CFTR. In addition to impaired trafficking, the mutation results in defective channel gating. Together, the reduced number of channels in the membrane and the defective gating lead to reduced anion transport across epithelia leading to defective ion and fluid transport. (Quinton, P. M. (1990), FASEB J. 4: 2709-2727). Studies have shown, however, that the reduced numbers of ΔF508-CFTR in the membrane are functional, albeit less than wild-type CFTR. (Dalemans et al. (1991), Nature Lond. 354: 526-528; Denning et al., supra; Pasyk and Foskett (1995), J. Cell. Biochem. 270: 12347-50). In addition to ΔF508-CFTR, other disease causing mutations in CFTR that result in defective trafficking, synthesis, and/or channel gating could be up- or down-regulated to alter anion secretion and modify disease progression and/or severity.

Although CFTR transports a variety of molecules in addition to anions, it is clear that this role (the transport of anions) represents one element in an important mechanism of transporting ions and water across the epithelium. The other elements include the epithelial Na+ channel (“ENaC”), Na+/2Cl−/K+ co-transporter, Na+−K+−ATPase pump and the basolateral membrane K+ channels, that are responsible for the uptake of chloride into the cell.

These elements work together to achieve directional transport across the epithelium via their selective expression and localization within the cell. Chloride absorption takes place by the coordinated activity of ENaC and CFTR present on the apical membrane and the Na+−K+−ATPase pump and Cl− ion channels expressed on the basolateral surface of the cell. Secondary active transport of chloride from the luminal side leads to the accumulation of intracellular chloride, which can then passively leave the cell via Cl− channels, resulting in a vectorial transport. Arrangement of Na+/2Cl−/K+ co-transporter, Na+−K+− ATPase pump and the basolateral membrane K+ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

As discussed above, it is believed that the deletion of residue 508 in ΔF508-CFTR prevents the nascent protein from folding correctly, resulting in the inability of this mutant protein to exit the ER, and traffic to the plasma membrane. As a result, insufficient amounts of the mature protein are present at the plasma membrane and chloride transport within epithelial tissues is significantly reduced. In fact, this cellular phenomenon of defective ER processing of ABC transporters by the ER machinery has been shown to be the underlying basis not only for CF disease, but for a wide range of other isolated and inherited diseases.

There is a need for methods of treating ABC transporter/ENaC mediated diseases using such combination compositions comprising at least one modulator of ABC transporter activity and at least one inhibitor of ENaC activity.

There is a need for methods for modulating an ABC transporter activity and/or ENaC activity in an ex vivo cell membrane of a mammal.

There is a need for modulators of CFTR activity that can be used to modulate the activity of CFTR in the cell membrane of a mammal.

There is a need for methods for treating CFTR-mediated diseases using such modulators of CFTR activity.

There is a need for methods for treating ENaC-mediated diseases using such modulators, in particular, inhibitors of ENaC activity.

There is a need for methods of modulating CFTR activity in an ex vivo cell membrane of a mammal.

### SUMMARY

These and other needs are met by the present invention which is directed to a pharmaceutical composition comprising:

A. an epithelial sodium channel (ENaC) inhibitor; and

B. at least one ABC transporter modulator, the ABC transporter comprising:
I. a compound of Formula A:

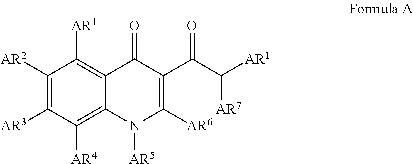

or pharmaceutically acceptable salts thereof, wherein:

Ar¹ is selected from:

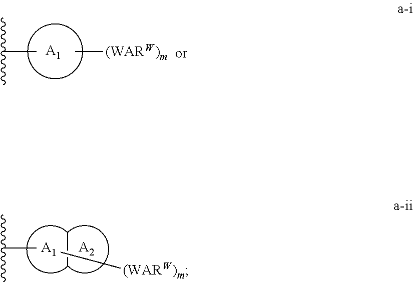

wherein ring A₁ is a 5-6 membered aromatic monocyclic ring having 0-4 heteroatoms independently selected from nitrogen, oxygen, or sulfur; or

A₁ and A₂, together, form an 8-14 membered aromatic, bicyclic or tricyclic aryl ring, wherein each ring contains 0-4 heteroatoms independently selected from nitrogen, oxygen, or sulfur; or
II. a compound of Formula B:

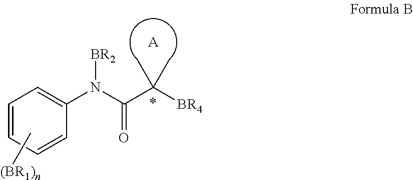

or a pharmaceutically acceptable salt thereof wherein: each BR₁ is an optionally substituted C₁₋₆ aliphatic, an optionally substituted aryl, an optionally substituted heteroaryl, an optionally substituted C₃₋₁₀ cycloaliphatic, or an optionally substituted 4 to 10 membered heterocycloaliphatic, carboxy [e.g., hydroxycarbonyl or alkoxycarbonyl], alkoxy, amido [e.g., aminocarbonyl], amino, halo, cyano, alkylsulfanyl, or hydroxy; provided that at least one BR₁ is an optionally substituted aryl or an optionally substituted heteroaryl and said BR₁ is attached to the 3- or 4-position of the phenyl ring; each BR₂ is hydrogen, an optionally substituted C₁₋₆ aliphatic, an optionally substituted C₃₋₆ cycloaliphatic, an optionally substituted phenyl, or an optionally substituted heteroaryl; each BR₄ is an optionally substituted aryl or an optionally substituted heteroaryl; each n is 1, 2, 3, 4 or 5; and ring A is an optionally substituted cycloaliphatic or an optionally substituted heterocycloaliphatic where the atoms of ring A adjacent to C* are carbon atoms, and each of which is optionally substituted with 1, 2, or 3 substituents; or
III. a compound of Formula C:

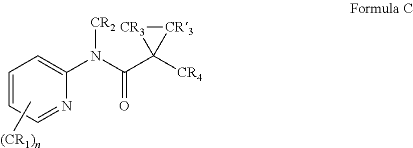

or a pharmaceutically acceptable salt thereof, wherein each CR₁ is a an optionally substituted C₁-C₆ aliphatic, an optionally substituted aryl, an optionally substituted heteroaryl, an optionally substituted 3 to 10 membered cycloaliphatic, an optionally substituted 3 to 10 membered heterocycloaliphatic, carboxy [e.g., hydroxycarbonyl or alkoxycarbonyl], amido, amino, halo, or hydroxy, provided that at least one CR₁ is an optionally substituted aryl or an optionally substituted heteroaryl attached to the 5- or 6-position of the pyridyl ring, each CR₂ is hydrogen, an optionally substituted C₁₋₆ aliphatic, an optionally substituted C₃₋₆ cycloaliphatic, an optionally substituted phenyl, or an optionally substituted heteroaryl, each CR₃ and CR′₃ together with the carbon atom to which they are attached form an optionally substituted C₃₋₇ cycloaliphatic or an optionally substituted heterocycloaliphatic, each CR₄ is an optionally substituted aryl or an optionally substituted heteroaryl, each n is 1-4; or
IV. a compound of Formula D:

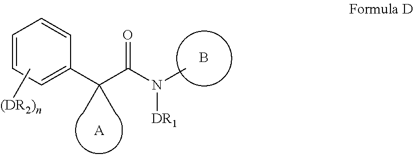

or a pharmaceutically acceptable salt thereof, wherein DR₁ is —Z^{A}DR₄, and wherein each Z^{A} is independently a bond or an optionally substituted branched or straight C₁₋₆ aliphatic chain wherein up to two carbon units of Z^{A} are optionally and independently replaced by —CO—, —CS—, —CONDR^{A}—, —CONDR^{A}NDR^{A}—, —CO₂—, —OCO—, —NDR^{A}CO₂—, —O—, —NDR^{A}CONDR^{A}—, —OCONDR^{A}—, —NDR^{A}NDR^{A}—, —NDR^{A}CO—, —S—, —SO—, —SO₂—, —NDR^{A}—, —SO₂NDR^{A}—, —NDR^{A}SO₂—, or —NDR^{A}SO₂NDR^{A}—,

Each DR₄ is independently DR^{A}, halo, —OH, —NH₂, —NO₂, —CN, or —OCF₃, each DR^{A} is independently hydrogen, an optionally substituted aliphatic, an optionally substituted cycloaliphatic, an optionally substituted heterocycloaliphatic, an optionally substituted aryl, or an optionally substituted heteroaryl, DR₂ is —Z^{B}DR₅, and wherein each Z^{B} is independently a bond or an optionally substituted branched or straight C₁₋₆ aliphatic chain wherein up to two carbon units of Z^{B} are optionally and independently replaced by —CO—, —CS—, —CONDR^{B}—, —CONDR^{B}NDR^{B}—, —CO₂—, —OCO—, —NDR^{B}CO₂—, —O—, —NDR^{B}CONDR^{B}—, —OCONDR^{B}—, —NDR^{B}NDR^{B}—, —NDR^{B}CO—, —S—, —SO—, —SO₂—, —NDR^{B}—, —SO₂NDR^{B}—, —NDR^{B}SO₂—, or —NDR^{B}SO₂NDR^{B}—, each DR₅ is independently DR^{B}, halo, —OH, —NH₂, —NO₂, —CN, —CF₃, or —OCF₃,

Each DR^{B} is independently hydrogen, an optionally substituted aliphatic, an optionally substituted cycloaliphatic, an optionally substituted heterocycloaliphatic, an optionally substituted aryl, or an optionally substituted heteroary, and wherein any two adjacent DR₂ groups together with the atoms to which they are attached form an optionally substituted carbocycle or an optionally substituted heterocycle,

wherein ring A is an optionally substituted 3-7 membered monocyclic ring having 0-3 heteroatoms selected from N, O, and S and ring B is a group having formula DIa.

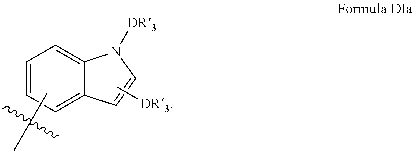

Each DR₃ and DR′₃ is independently —Z^{C}DR₆, where each Z^{C} is independently a bond or an optionally substituted branched or straight C₁₋₆ aliphatic chain wherein up to two carbon units of Z^{C} are optionally and independently replaced by —CO—, —CS—, —CONDR^{C}—, —CONDR^{c}NDR^{c}—, —CO₂—, —OCO—, —NDR^{c}CO₂—, —O—, —NDR^{C}CONDR^{C}—, —OCONDR^{C}—, —NDR^{C}NDR^{C}—, —NDR^{C}CO—, —S—, —SO—, —SO₂—, —NDR^{C}—, —SO₂NDR^{C}—, —NDR^{C}SO₂—, or —NDR^{C}SO₂NDR^{C}—. Each DR₆ is independently DR^{C}, halo, —OH, —NH₂, —NO₂, —CN, or —OCF₃. Each DR^{C} is independently hydrogen, an optionally substituted aliphatic, an optionally substituted cycloaliphatic, an optionally substituted heterocycloaliphatic, an optionally substituted aryl, or an optionally substituted heteroaryl. Alternatively, any two adjacent DR₃ groups together with the atoms to which they are attached form an optionally substituted carbocycle or an optionally substituted heterocycle, or DR′₃ and an adjacent DR₃, i.e., attached to the 2 position of the indole of formula Ia, together with the atoms to which they are attached form an optionally substituted heterocycle.

In some embodiments, the pharmaceutical composition comprises:

A. an epithelial sodium channel (ENaC) inhibitor; and at least one of:

B. a compound of Formula A1;

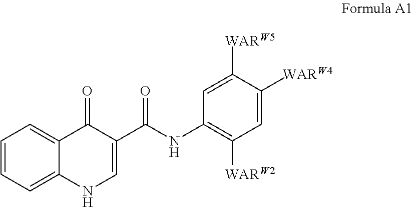

or pharmaceutically acceptable salts thereof, wherein:

Each of WAR^{W2} and WAR^{W4} is independently selected from CN, CF₃, halo, C₂₋₆ straight or branched alkyl, C₃₋₁₂ membered cycloaliphatic, phenyl, a 5-10 membered heteroaryl or 3-7 membered heterocyclic, wherein said heteroaryl or heterocyclic has up to 3 heteroatoms selected from O, S, or N, wherein said WAR^{W2} and WAR^{W4} is independently and optionally substituted with up to three substituents selected from —OAR′, —CF₃, —OCF₃, SDR′, S(O)AR′, SO₂AR′, —SCF₃, halo, CN, —COOAR′, —COAR′, —O(CH₂)₂N(AR′)₂, —O(CH₂)N(AR′)₂, —CON(AR′)₂, —(CH₂)₂OAR′, —(CH₂)OAR′, —CH₂CN, optionally substituted phenyl or phenoxy, —N(AR′)₂, —NAR′C(O)OAR′, —NAR′C(O)AR′, —(CH₂)₂N(AR′)₂, or —(CH₂)N(AR′)₂; WR^{W5} is selected from hydrogen, —OCF₃, —CF₃, —OH, —OCH₃, —NH₂, —CN, —CHF₂, —NHR′, —N(AR′)₂, —NHC(O)AR′, —NHC(O)OAR′, —NHSO₂AR′, —CH₂OH, —CH₂N(AR′)₂, —C(O)OAR′, —SO₂NHAR′, —SO₂N(AR′)₂, or —CH₂NHC(O)OAR′; and

Each AR′ is independently selected from an optionally substituted group selected from a C₁₋₈ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or two occurrences of AR′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur;

provided that:

i) WAR^{W2} and WAR^{W4} are not both —Cl;

WAR^{W2}, WAR^{W4} and WAR^{W5} are not —OCH₂CH₂Ph, —OCH₂CH₂(2-trifluoromethyl-phenyl), —OCH₂CH₂-(6,7-dimethoxy-1,2,3,4-tetrahydroisoquinolin-2-yl), or substituted 1H-pyrazol-3-yl; or
C. a compound of Formula C1

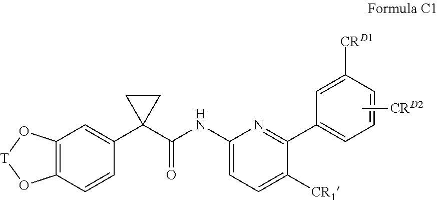

or pharmaceutically acceptable salts thereof, wherein:

### T is —CH₂—, —CH₂CH₂—, —CF₂—, —C(CH₃)₂—, or —C(O)—;

CR₁′ is H, C₁₋₆ aliphatic, halo, CF₃, CHF₂, O(C₁₋₆ aliphatic); and

### CR^{D1} or CR^{D2} is Z^{D}CR₉

wherein:
Z^{D} is a bond, CONH, SO₂NH, SO₂N(C₁₋₆ alkyl), CH₂NHSO₂, CH₂N(CH₃)SO₂, CH₂NHCO, COO, SO₂, or CO; and CR₉ is H, C₁₋₆ aliphatic, or aryl; or
D. a compound of Formula D1

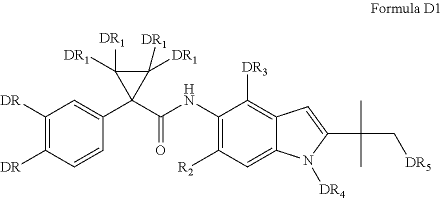

or pharmaceutically acceptable salts thereof, wherein:
DR is H, OH, OCH₃ or two R taken together form —OCH₂O— or —OCF₂O—;
DR₄ is H or alkyl;

### DR₅ is H or F;

### DR₆ is H or CN;

DR₇ is H, —CH₂CH(OH)CH₂OH, —CH₂CH₂N⁺(CH₃)₃, or —CH₂CH₂OH; DR₈ is H, OH, —CH₂CH(OH)CH₂OH, —CH₂OH, or DR₇ and DR₈ taken together form a five membered ring.

In some embodiments, the at least one ENaC inhibitor comprises a compound of Formula E

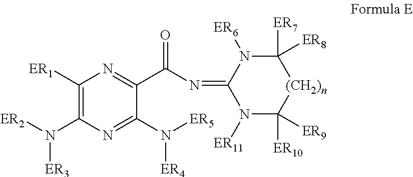

In one aspect, the pharmaceutical composition comprises an inhibitor of ENaC activity and at least one compound of Formula AI, or Formula CI or Formula DI.

In another aspect, the pharmaceutical composition comprises an inhibitor of ENaC activity and Compound 1.

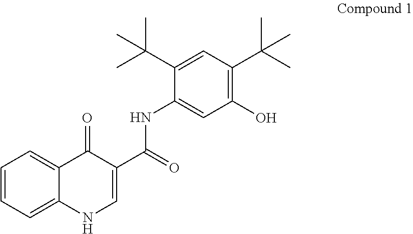 [SMILES: CC(C)(C)c1cc(C(C)(C)C)c(NC(=O)c2c[nH]c3ccccc3c2=O)cc1O]

In another aspect, the pharmaceutical composition comprises an inhibitor of ENaC activity and Compound 2.

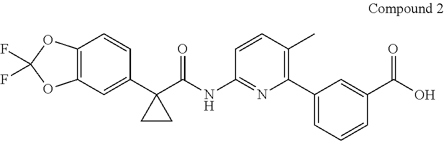 [SMILES: Cc1ccc(NC(=O)C2(c3ccc4c(c3)OC(F)(F)O4)CC2)nc1-c1cccc(C(=O)O)c1]

In another aspect, the pharmaceutical composition comprises an inhibitor of ENaC activity and Compound 3.

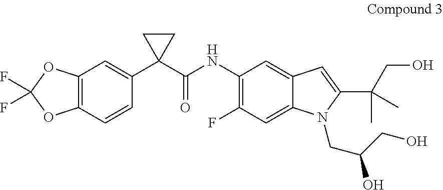 [SMILES: CC(C)(CO)c1cc2cc(NC(=O)C3(c4ccc5c(c4)OC(F)(F)O5)CC3)c(F)cc2n1C[C@H](O)CO]

In another aspect, the invention is directed to a composition, preferably a pharmaceutical composition comprising at least one component from: Column A of Table I, or Column B of Table I, or Column C of Table I, or Column D of Table I, in combination with at least one ENaC inhibitor component from Column E of Table I. These components are described in the corresponding sections of the following pages as embodiments of the invention. For convenience, Table I recites the section number and corresponding heading title of the embodiments of the compounds.

TABLE I
Compounds

| Column A | | Column B | | Column C | | Column D | | Column E | |
|---|---|---|---|---|---|---|---|---|---|
| Embodiments | | Embodiments | | Embodiments | | Embodiments | | Embodiments | |
| Section | Heading | Section | Heading | Section | Heading | Section | Heading | Section | Heading |
| II.A.1. | Compound | II.B.1. | Compound | II.C.1. | Compound | II.D.1. | Compound | II.E.1. | ENAC |
| | of Formula | | of Formula | | of Formula | | of Formula | | Compounds |
| | A | | B | | C | | D | | |
| II.A.2 | Compound | II.B.2 | Compound | II.C.2 | Compound | II.D.2 | Compound | II.E.2 | Compound of |
| | of Formula | | of Formula | | of Formula | | of Formula | | Formula E |
| | A1 | | B1 & B2 | | C1 | | D1 | | |
| II.A.3. | Compound | | | II.C.3. | Compound | II.D.3. | Compound | | |
| | 1 | | | | 2 | | 3 | | |

For example, the embodiments of the compounds of Formula A are disclosed in section II.A.1. of this specification.

For another example, the embodiments of the compounds of Formula B are disclosed in section II.B.1. of this specification.

For another example, the embodiments of the compounds of Formula C are disclosed in section II.C.1. of this specification.

For another example, the embodiments of the compounds of Formula D are disclosed in section II.D.1. of this specification.

For another example, the embodiments of the ENaC compounds of Formula E are illustratively described in section II.E.2. of this specification.

In one embodiment based on Table I, the Column A component is Compound 1, the Column C Component is Compound 2, and the Column D Component is Compound 3.

In another aspect, the invention is directed to method of treating a CFTR mediated disease in a human comprising administering to the human, an effective amount of a pharmaceutical composition comprising an ENaC inhibitor component of Column E and an ABC modulator component selected from at least one of Columns A, or B, or C, or D according to Table I.

It has now been found that pharmaceutically acceptable compositions of the present invention, include the combination of a modulator of ABC transporter activity or cAMP/ATP-mediated anion channel, Cystic Fibrosis Transmembrane Conductance Regulator (“CFTR”) and a modulator of ENaC activity.

In another aspect, the combination compounds are provided to treat a variety of diseases and disorders mediated by ABC transporters and/or ENaC. The combination composition can include a modulator of an ABC transporter corresponding to one or more of Formulas I, II and III and an inhibitor of ENaC, for example, compounds of Formula IV. While the methods for treating said variety of diseases and disorders mediated by ABC transporters and/or ENaC comprises a combination of a an ENaC inhibitor component of Column D and an ABC modulator component selected from at least one of Columns A, B, C, or D according to Table I, the individual active agents can be administered in a single dose unit, as separate dosage units, administered simultaneously, or may be administered sequentially, optionally within a specified time period of the other's administration.

In another aspect, the invention is directed to method of treating a CFTR mediated disease in a human comprising administering to the human an effective amount of a ENaC inhibitor component of Column E and at least one of Compounds 1, 2, or 3 according to Table I.

Methods are provided to treat CF and other chronic diseases mediated by dysregulation or dysfunctional ABC transporter activity or cAMP/ATP-mediated anion channel and epithelial sodium channel (ENaC) activity using the pharmaceutical compositions described herein.

In another aspect, the invention is directed to a kit for the treatment of a CFTR mediated disease in a human, the kit comprising an ENaC inhibitor component of Column E and an ABC modulator component selected from at least one of Columns A, or B, or C, or D according to Table I, and optionally, instructions for preparing and administering a pharmaceutical composition for the treatment of said disease.

In another aspect, the invention is directed to a kit for the treatment of a CFTR mediated disease in a human, the kit comprising an ENaC inhibitor component of Formula E and an ABC modulator component selected from at least one of Formulas A1, or B1, or C1, or D1 according to Table I, and optionally, instructions for preparing and administering a pharmaceutical composition for the treatment of said disease.

Various components listed in Table I have been disclosed and can be found in have been disclosed and can be found in U.S. Pat. No. 7,691,902 (US 2008/0044355), U.S. Pat. No. 7,671,221 (US 2008/0009524), U.S. Pat. No. 7,741,321, U.S. Pat. No. 7,645,789, U.S. Pat. No. 7,495,103, U.S. Pat. No. 7,776,905, U.S. Pat. No. 7,659,268, U.S. Patent Application publications US 2007/0244159A1, US 2008/0113985A1, US 2008/0019915A1, US 2008/0306062A1, US 2006/0074075A1 and US 2009/0131492A1 the contents of all of the above published patent applications and patents are incorporated herein by reference in their entireties.

### DETAILED DESCRIPTION

The invention relates to a combination of active agents, particularly a pharmaceutical combination, such as a combined preparation or pharmaceutical composition, respectively, which comprises 1) a modulator of ATP-Binding Cassette (“ABC”) transporters or fragments thereof, including Cystic Fibrosis Transmembrane Conductance Regulator (“CFTR”) and 2) an epithelial sodium channel inhibitor (“ENaC”), for simultaneous, separate or sequential use, especially in the prevention, delay of progression or treatment of conditions mediated by CFTR and ENaC, conditions directly caused by ABC Transporter and/or CFTR activities and alleviation of symptoms of diseases not directly caused by ABC Transporter and/or CFTR anion channel activities.

Examples of diseases whose symptoms may be affected by ABC Transporter e.g. CFTR and/or ENaC activity include, but are not limited to, CF, Hereditary emphysema, Hereditary hemochromatosis, Coagulation-Fibrinolysis deficiencies, such as Protein C deficiency, Type 1 hereditary angioedema, Lipid processing deficiencies, such as Familial hypercholesterolemia, Type 1 chylomicronemia, Abetalipoproteinemia, Lysosomal storage diseases, such as I-cell disease/Pseudo-Hurler, Mucopolysaccharidoses, Sandhof/Tay-Sachs, Crigler-Najjar type II, Polyendocrinopathy/Hyperinsulemia, Diabetes mellitus, Laron dwarfism, Myleoperoxidase deficiency, Primary hypoparathyroidism, Melanoma, Glycanosis CDG type 1, Hereditary emphysema, Congenital hyperthyroidism, Osteogenesis imperfecta, Hereditary hypofibrinogenemia, ACT deficiency, Diabetes insipidus (DI), Neurophysiol DI, Nephrogenic DI, Charcot-Marie Tooth syndrome, Perlizaeus-Merzbacher disease, neurodegenerative diseases such as Alzheimer's disease, Parkinson's disease, Amyotrophic lateral sclerosis, Progressive supranuclear palsy, Pick's disease, several polyglutamine neurological disorders such as Huntington, Spinocerebullar ataxia type I, Spinal and bulbar muscular atrophy, Dentatorubal pallidoluysian, and Myotonic dystrophy, as well as Spongiform encephalopathies, such as Hereditary Creutzfeldt-Jakob disease, Fabry disease, Straussler-Scheinker syndrome, COPD, dry-eye disease, and Sjogren's disease

In some embodiments, the present invention also provides for the use of such combination, for the preparation of a pharmaceutical composition for the prevention, delay, of progression or treatment of such conditions, diseases and disorders; providing kits comprising such combination for the treatment of a mammal.

### DEFINITIONS

As used herein, the following definitions shall apply unless otherwise indicated.

The term “ABC-transporter” as used herein means an ABC-transporter protein or a fragment thereof comprising at least one binding domain, wherein said protein or fragment thereof is present in vivo or in vitro. The term “binding domain” as used herein means a domain on the ABC-transporter that can bind to a modulator. See, e.g., Hwang, T. C. et al., J. Gen. Physiol. (1998): 111(3), 477-90.

The term “CFTR” as used herein means cystic fibrosis transmembrane conductance regulator or a mutation thereof capable of regulator activity, including, but not limited to, ΔF508 CFTR and G551D CFTR (see, e.g., http://www.genet.sickkids.on.ca/cftr/, for CFTR mutations).

The term “modulating” as used herein means increasing or decreasing, e.g. activity, by a measurable amount. Compounds that modulate ABC Transporter activity, such as CFTR activity, by increasing the activity of the ABC Transporter, e.g., a CFTR anion channel, are called agonists. Compounds that modulate ABC Transporter activity, such as CFTR activity, by decreasing the activity of the ABC Transporter, e.g., CFTR anion channel, are called antagonists. An agonist interacts with an ABC Transporter, such as CFTR anion channel, to increase the ability of the receptor to transduce an intracellular signal in response to endogenous ligand binding. An antagonist interacts with an ABC Transporter, such as CFTR, and competes with the endogenous ligand(s) or substrate(s) for binding site(s) on the receptor to decrease the ability of the receptor to transduce an intracellular signal in response to endogenous ligand binding.

The phrase “treating or reducing the severity of an ABC Transporter mediated disease” refers both to treatments for diseases that are directly caused by ABC Transporter and/or CFTR activities and alleviation of symptoms of diseases not directly caused by ABC Transporter and/or CFTR anion channel activities. Examples of diseases whose symptoms may be affected by ABC Transporter and/or CFTR activity include, but are not limited to, Cystic fibrosis, Hereditary emphysema, Hereditary hemochromatosis, Coagulation-Fibrinolysis deficiencies, such as Protein C deficiency, Type 1 hereditary angioedema, Lipid processing deficiencies, such as Familial hypercholesterolemia, Type 1 chylomicronemia, Abetalipoproteinemia, Lysosomal storage diseases, such as I-cell disease/Pseudo-Hurler, Mucopolysaccharidoses, Sandhof/Tay-Sachs, Crigler-Najjar type II, Polyendocrinopathy/Hyperinsulemia, Diabetes mellitus, Laron dwarfism, Myleoperoxidase deficiency, Primary hypoparathyroidism, Melanoma, Glycanosis CDG type 1, Hereditary emphysema, Congenital hyperthyroidism, Osteogenesis imperfecta, Hereditary hypofibrinogenemia, ACT deficiency, Diabetes insipidus (DI), Neurophyseal DI, Neprogenic DI, Charcot-Marie Tooth syndrome, Perlizaeus-Merzbacher disease, neurodegenerative diseases such as Alzheimer's disease, Parkinson's disease, Amyotrophic lateral sclerosis, Progressive supranuclear plasy, Pick's disease, several polyglutamine neurological disorders such as Huntington, Spinocerebullar ataxia type I, Spinal and bulbar muscular atrophy, Dentatorubal pallidoluysian, and Myotonic dystrophy, as well as Spongiform encephalopathies, such as Hereditary Creutzfeldt-Jakob disease, Fabry disease, Straussler-Scheinker syndrome, COPD, dry-eye disease, and Sjogren's disease.

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

For the purposes of this invention formula specific R groups have been designated a preceding letter representing the column in which they are recited. For example, an R¹ group that is specific for Formula A1 has been written as AR¹, an R^{A} group in Formula D is designated DR^{A} to distinguish from other R^{A} groups used in other Formulas from the other columns, and so on and so forth.

As used herein the term “aliphatic” encompasses the terms alkyl, alkenyl, alkynyl, each of which being optionally substituted as set forth below.

As used herein, an “alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino], amino [e.g., aliphaticamino, cycloaliphaticamino, or heterocycloaliphaticamino], sulfonyl [e.g., aliphaticsulfonyl], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, hydroxyalkyl, aralkyl, (alkoxyaryl)alkyl, (sulfonylamino)alkyl (such as (alkylsulfonylamino)alkyl), aminoalkyl, amidoalkyl, (cycloaliphatic)alkyl, cyanoalkyl, or haloalkyl.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as halo, cycloaliphatic, heterocycloaliphatic, aryl, heteroaryl, alkoxy, aroyl, heteroaroyl, acyl [e.g., (cycloaliphatic)carbonyl, or (heterocycloaliphatic)carbonyl], nitro, cyano, acyl [e.g., aliphaticcarbonyl, cycloaliphaticcarbonyl, arylcarbonyl, heterocycloaliphaticcarbonyl or heteroarylcarbonyl], amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, or aliphaticsulfonylamino], sulfonyl [e.g., alkylsulfonyl, cycloaliphaticsulfonyl, or arylsulfonyl], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy.

As used herein, an “alkynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl [e.g., aliphaticsulfanyl or cycloaliphaticsulfanyl], sulfinyl [e.g., aliphaticsulfinyl or cycloaliphaticsulfinyl], sulfonyl [e.g., aliphaticsulfonyl, aliphaticaminosulfonyl, or cycloaliphaticsulfonyl], amido [e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl], urea, thiourea, sulfamoyl, sulfamide, alkoxycarbonyl, alkylcarbonyloxy, cycloaliphatic, heterocycloaliphatic, aryl, heteroaryl, acyl [e.g., (cycloaliphatic)carbonyl or (heterocycloaliphatic)carbonyl], amino [e.g., aliphaticamino], sulfoxy, oxo, carboxy, carbamoyl, (cycloaliphatic)oxy, (heterocycloaliphatic)oxy, or (heteroaryl)alkoxy.

As used herein, an “amido” encompasses both “aminocarbonyl” and “carbonylamino”. These terms when used alone or in connection with another group refers to an amido group such as N(RXRY)—C(O)— or RYC(O)—N(RX)— when used terminally and —C(O)—N(RX)— or —N(RX)—C(O)— when used internally, wherein RX and RY are defined below. Examples of amido groups include alkylamido (such as alkylcarbonylamino or alkylcarbonylamino), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NRXRY wherein each of RX and RY is independently hydrogen, alkyl, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NRX—. RX has the same meaning as defined above.

As used herein, an “aryl” group used alone or as part of a larger moiety as in “aralkyl”, “aralkoxy”, or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic ring systems include benzofused 2-3 membered carbocyclic rings. For example, a benzofused group includes phenyl fused with two or more C4-8 carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphaticsulfonyl or aminosulfonyl]; sulfinyl [e.g., aliphaticsulfinyl or cycloaliphaticsulfinyl]; sulfanyl [e.g., aliphaticsulfanyl]; cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl [e.g., mono-, di (such as p,m-dihaloaryl), and (trihalo)aryl]; (carboxy)aryl [e.g., (alkoxycarbonyl)aryl, ((aralkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl or ((dialkyl)amino)aryl]; (cyanoalkyl)aryl; (alkoxy)aryl; (sulfamoyl)aryl [e.g., (aminosulfonyl)aryl]; (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C1-4 alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C1-4 alkyl group) that is substituted with an aryl group. Both “alkyl” and “aryl” have been defined above. An example of an aralkyl group is benzyl. An aralkyl is optionally substituted with one or more substituents such as aliphatic [e.g., alkyl, alkenyl, or alkynyl, including carboxyalkyl, hydroxyalkyl, or haloalkyl such as trifluoromethyl], cycloaliphatic [e.g., cycloalkyl or cycloalkenyl], (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido [e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino], cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, azacycloalkyl, or ((aminocarbonyl)cycloalkyl)cycloalkyl. A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, or bicyclo[3.3.1]nonenyl. A cycloalkyl or cycloalkenyl group can be optionally substituted with one or more substituents such as aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl [e.g., alkylsulfonyl and arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

As used herein, “cyclic moiety” includes cycloaliphatic, heterocycloaliphatic, aryl, or heteroaryl, each of which has been defined previously.

As used herein, the term “heterocycloaliphatic” encompasses a heterocycloalkyl group and a heterocycloalkenyl group, each of which being optionally substituted as set forth below.

As used herein, a “heterocycloalkyl” group refers to a 3-10 membered mono- or bicyclic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. A monocyclic heterocycloalkyl group can be fused with a phenyl moiety such as tetrahydroisoquinoline. A “heterocycloalkenyl” group, as used herein, refers to a mono- or bicyclic (e.g., 5- to 10-membered mono- or bicyclic) non-aromatic ring structure having one or more double bonds, and wherein one or more of the ring atoms is a heteroatom (e.g., N, O, or S). Monocyclic and bicycloheteroaliphatics are numbered according to standard chemical nomenclature.

A heterocycloalkyl or heterocycloalkenyl group can be optionally substituted with one or more substituents such as aliphatic [e.g., alkyl, alkenyl, or alkynyl], cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], nitro, cyano, halo, hydroxy, mercapto, sulfonyl [e.g., alkylsulfonyl or arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophenyl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pyranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphaticsulfonyl or aminosulfonyl]; sulfinyl [e.g., aliphaticsulfinyl]; sulfanyl [e.g., aliphaticsulfanyl]; nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl [e.g., mono- and di-(halo)heteroaryl]; (carboxy)heteroaryl [e.g., (alkoxycarbonyl)heteroaryl]; cyanoheteroaryl; aminoheteroaryl [e.g., ((alkylsulfonyl)amino)heteroaryl and ((dialkyl)amino)heteroaryl]; (amido)heteroaryl [e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl]; (cyanoalkyl)heteroaryl; (alkoxy)heteroaryl; (sulfamoyl)heteroaryl [e.g., (aminosulfonyl)heteroaryl]; (sulfonyl)heteroaryl [e.g., (alkylsulfonyl)heteroaryl]; (hydroxyalkyl)heteroaryl; (alkoxyalkyl)heteroaryl; (hydroxy)heteroaryl; ((carboxy)alkyl)heteroaryl; [((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl [e.g., (alkylcarbonyl)heteroaryl]; (alkyl)heteroaryl, and (haloalkyl)heteroaryl [e.g., trihaloalkylheteroaryl].

A “heteroaraliphatic (such as a heteroaralkyl group) as used herein, refers to an aliphatic group (e.g., a C1-4 alkyl group) that is substituted with a heteroaryl group. “Aliphatic,” “alkyl,” and “heteroaryl” have been defined above.

A “heteroaralkyl” group, as used herein, refers to an alkyl group (e.g., a C1-4 alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, a “carbamoyl” group refers to a group having the structure —O—CO—NR^{X}R^{Y} or —NR^{X}—CO—O—R^{Z} wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X} when used as a terminal group; or —OC(O)— or —C(O)O— when used as an internal group.

As used herein, a “haloaliphatic” group refers to an aliphatic group substituted with 1-3 halogen. For instance, the term haloalkyl includes the group —CF₃.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —S(O)₂—NR^{X}R^{Y} or —NR^{X}—S(O)₂—R^{Z} when used terminally; or —S(O)₂—NR^{X}— or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include alkylsulfanyl.

As used herein a “sulfinyl” group refers to —S(O)—R^{X} when used terminally and —S(O)— when used internally, wherein R^{X} has been defined above.

As used herein, a “sulfonyl” group refers to —S(O)₂—R^{X} when used terminally and —S(O)₂— when used internally, wherein R^{X} has been defined above.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refer to —C(O)—.

As used herein, an “oxo” refers to ═O.

As used herein, an “aminoalkyl” refers to the structure (R^{X}R^{Y})N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidino” group refers to the structure —N═C(N(R^{X}R^{Y}))N(R^{X}R^{Y}) wherein R^{X} and R^{Y} have been defined above.

As used herein, the term “amidino” group refers to the structure —C═(NR^{X})N(R^{X}R^{Y}) wherein R^{X} and R^{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent to at the end of the substituent bound to the rest of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, the term “amidino” group refers to the structure —C═(NR^{X})N(R^{X}R^{Y}) wherein R^{X} and R^{Y} have been defined above.

As used herein, “cyclic group” includes mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicaliphatic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-6. A branched aliphatic chain is a straight aliphatic chain that is substituted with one or more aliphatic groups. A branched aliphatic chain has the structure —[CHQ]ᵥ- where Q is hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, R₃, and R₄, and other variables contained therein formulae I encompass specific groups, such as alkyl and aryl. Unless otherwise noted, each of the specific groups for the variables R₁, R₂, R₃, and R₄, and other variables contained therein can be optionally substituted with one or more substituents described herein. Each substituent of a specific group is further optionally substituted with one to three of halo, cyano, oxoalkoxy, hydroxy, amino, nitro, aryl, haloalkyl, and alkyl. For instance, an alkyl group can be substituted with alkylsulfanyl and the alkylsulfanyl can be optionally substituted with one to three of halo, cyano, oxoalkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an effective amount is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970). As used herein, “patient” refers to a mammal, including a human.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### I. Compositions

The compositions of the present invention includes a combination of at least one modulator of ABC transporter activity, for example, a modulator of CFTR recited below in Columns A, B, C, and D and one compound that blocks, suppresses or inhibits the activity of ENaC, recited below in Column E. In another aspect, the invention is directed to a pharmaceutical composition comprising at least one compound selected from Formulas A, B, C, or D and one compound from Formula E from Columns A-E of Table I. These components are described in the corresponding sections of the following pages as embodiments of the invention. For convenience, Table I recites the section number and corresponding heading title of the embodiments of the formulas and compounds.

TABLE I
Compounds

| Column A | | Column B | | Column C | | Column D | | Column E | |
|---|---|---|---|---|---|---|---|---|---|
| Embodiments | | Embodiments | | Embodiments | | Embodiments | | Embodiments | |
| Section | Heading | Section | Heading | Section | Heading | Section | Heading | Section | Heading |
| II.A.1. | Compound | II.B.1. | Compound | II.C.1. | Compound | II.D.1. | Compound | II.E.1. | ENAC |
| | of Formula | | of Formula | | of Formula | | of Formula | | Compounds |
| | A | | B | | C | | D | | |
| II.A.2 | Compound | II.B.2 | Compound | II.C.2 | Compound | II.D.2 | Compound | II.E.2 | Compound of |
| | of Formula | | of Formula | | of Formula | | of Formula | | Formula E |
| | A1 | | B1 & B2 | | C1 | | D1 | | |
| II.A.3. | Compound | | | II.C.3. | Compound | II.D.3. | Compound | | |
| | 1 | | | | 2 | | 3 | | |

Subgeneric formulas of Formulas A-E are provided as Formula A1, Formula B1 & B2, Formula C1, Formula D1, and Formula E1.

Various components listed in Table I above have been disclosed and can be found in U.S. Pat. No. 7,691,902 (US 2008/0044355), U.S. Pat. No. 7,671,221 (US 2008/0009524), US 2007/0244159A1, U.S. Pat. No. 7,645,789, U.S. Pat. No. 7,495,103, U.S. Pat. No. 7,553,855, U.S. Pat. App. Pub. Nos: 2010-0074949, U.S. 2008/0113985, U.S. 2008/0019915, U.S. 2008/0306062, U.S. 2009/0170905, U.S. 2009/0176839 and U.S. 2010/00847490 the contents of which are incorporated herein by reference in their entireties.

### II.A Embodiments of Column a Compounds

The modulators of ABC transporter activity in Column A are fully described and exemplified in U.S. Pat. No. 7,495,103 and US Application Publication US 2010/0184739 which are commonly assigned to the Assignee of the present invention. All of the compounds recited in the above patents are useful in the present invention and are hereby incorporated into the present disclosure in their entirety. In some embodiments, the compositions, including pharmaceutical compositions of the present invention, include at least one component of Column A in combination with an ENaC inhibitor component of Column E.

### II.A.1 Compounds of Formula A

It has now been found that compounds of this invention, and pharmaceutically acceptable compositions thereof, are useful as modulators of ABC transporter activity. These compounds have the general Formula A

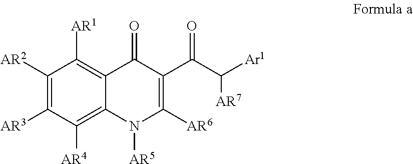

or a pharmaceutically acceptable salt thereof, wherein AR′, AR², AR³, AR⁴, AR⁵, AR⁶, AR⁷, and Ar¹ are described generally and in classes and subclasses below.

One compound of the combined composition can include a compound provided wherein, Ar¹ is selected from:

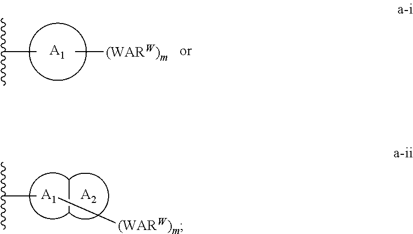

wherein ring A₁ 5-6 membered aromatic monocyclic ring having 0-4 heteroatoms independently selected from nitrogen, oxygen, or sulfur; or

A₁ and A₂, together, is an 8-14 aromatic, bicyclic or tricyclic aryl ring, wherein each ring contains 0-4 heteroatoms independently selected from nitrogen, oxygen, or sulfur.

In some embodiments, A₁ is an optionally substituted 6 membered aromatic ring having 0-4 heteroatoms, wherein said heteroatom is nitrogen. In some embodiments, A₁ is an optionally substituted phenyl. Or, A₁ is an optionally substituted pyridyl, pyrimidinyl, pyrazinyl or triazinyl. Or, A₁ is an optionally substituted pyrazinyl or triazinyl. Or, Aₜ is an optionally substituted pyridyl.

In some embodiments, A₁ is an optionally substituted 5-membered aromatic ring having 0-3 heteroatoms, wherein said heteroatom is nitrogen, oxygen, or sulfur. In some embodiments, A₁ is an optionally substituted 5-membered aromatic ring having 1-2 nitrogen atoms. In one embodiment, A₁ is an optionally substituted 5-membered aromatic ring other than thiazolyl.

In some embodiments, A₂ is an optionally substituted 6 membered aromatic ring having 0-4 heteroatoms, wherein said heteroatom is nitrogen. In some embodiments, A₂ is an optionally substituted phenyl. Or, A₂ is an optionally substituted pyridyl, pyrimidinyl, pyrazinyl, or triazinyl.

In some embodiments, A₂ is an optionally substituted 5-membered aromatic ring having 0-3 heteroatoms, wherein said heteroatom is nitrogen, oxygen, or sulfur. In some embodiments, A₂ is an optionally substituted 5-membered aromatic ring having 1-2 nitrogen atoms. In certain embodiments, A₂ is an optionally substituted pyrrolyl.

In some embodiments, A₂ is an optionally substituted 5-7 membered saturated or unsaturated heterocyclic ring having 1-3 heteroatoms independently selected from nitrogen, sulfur, or oxygen. Exemplary such rings include piperidyl, piperazyl, morpholinyl, thiomorpholinyl, pyrrolidinyl, tetrahydrofuranyl, etc.

In some embodiments, A₂ is an optionally substituted 5-10 membered saturated or unsaturated carbocyclic ring. In one embodiment, A₂ is an optionally substituted 5-10 membered saturated carbocyclic ring. Exemplary such rings include cyclohexyl, cyclopentyl, etc.

In some embodiments, ring A₂ is selected from:

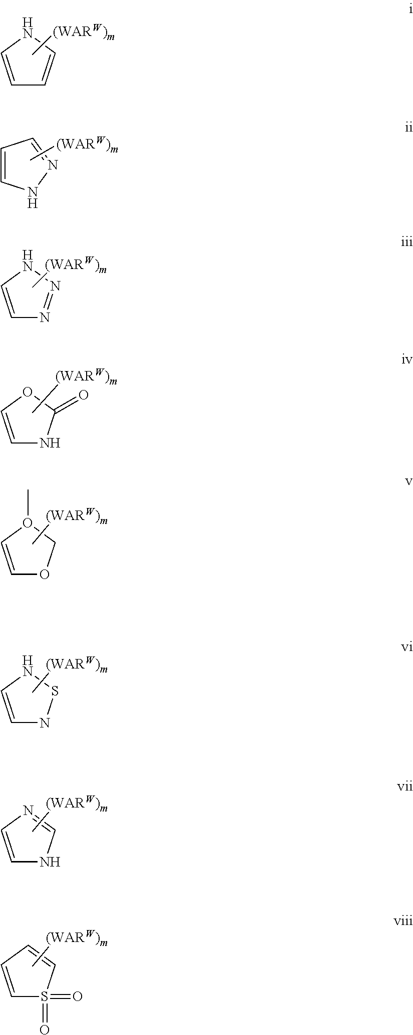

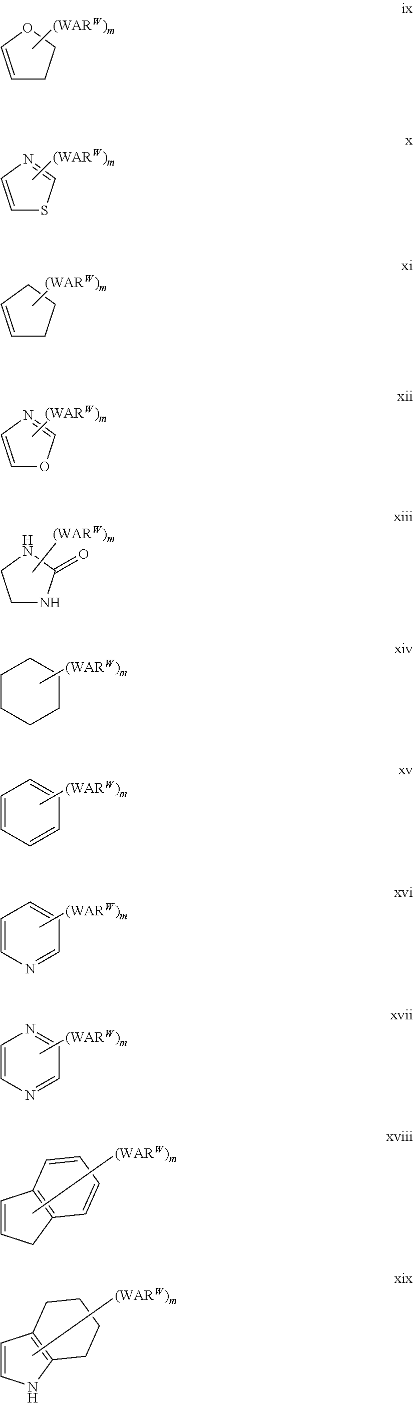

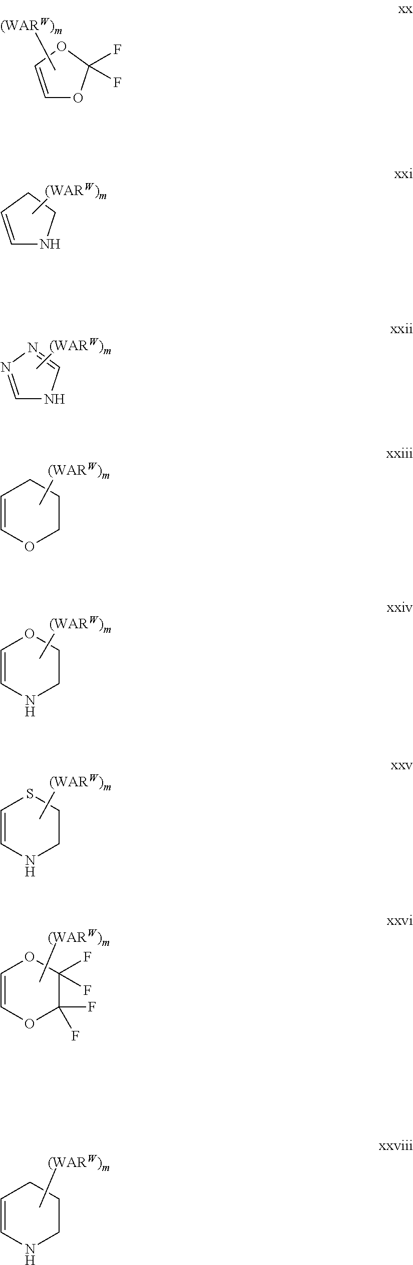

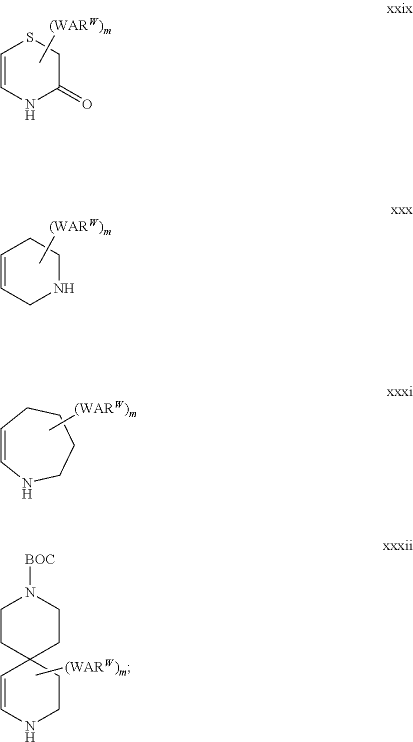

wherein ring A₂ is fused to ring A, through two adjacent ring atoms.

In other embodiments, W is a bond or is an optionally substituted C₁₋₆ alkylidene chain wherein one or two methylene units are optionally and independently replaced by O, NAR′, S, SO, SO₂, or COO, CO, SO₂NAR′, NAR′SO₂, C(O)NAR′, NAR′C(O), OC(O), OC(O)NAR′, and AR^{W} is AR′ or halo. In still other embodiments, each occurrence of WAR^{W} is independently —C1-C3 alkyl, C1-C3 perhaloalkyl, —O(C1-C3alkyl), —CF₃, —OCF₃, —SCF₃, —F, —Cl, —Br, or —COOAR′, —COAR′, —O(CH₂)₂N(AR′)(AR′), —O(CH₂)N(AR′)(AR′), —CON(AR′)(AR′), —(CH₂)₂OAR′, —(CH₂)OAR′, optionally substituted monocyclic or bicyclic aromatic ring, optionally substituted arylsulfone, optionally substituted 5-membered heteroaryl ring, —N(AR′)(AR′), —(CH₂)₂N(AR′)(AR′), or —(CH₂)N(AR′)(AR′).

In some embodiments, m is 0. Or, m is 1. Or, m is 2. In some embodiments, m is 3. In yet other embodiments, m is 4.

In one embodiment, AR⁵ is X-AR^{X}. In some embodiments AR⁵ is hydrogen. Or, AR⁵ is an optionally substituted C₁₋₈ aliphatic group. In some embodiments, AR⁵ is optionally substituted C₁₋₄ aliphatic. Or, AR^{S} is benzyl.

In some embodiments AR⁶ is hydrogen. Or, AR⁶ is an optionally substituted C₁₋₈ aliphatic group. In some embodiments, AR⁶ is optionally substituted C₁₋₄ aliphatic. In certain other embodiments, AR⁶ is —(O—C₁₋₄ aliphatic) or —(S—C₁₋₄ aliphatic). Preferably, AR⁶ is —OMe or —SMe. In certain other embodiments, AR₆ is CF₃.

In one embodiment of the present invention, AR¹, AR², AR³, and AR⁴ are simultaneously hydrogen. In another embodiment, AR⁶ and AR⁷ are both simultaneously hydrogen.

In another embodiment of the present invention, AR¹, AR², AR³, AR⁴, and AR⁵ are simultaneously hydrogen. In another embodiment of the present invention, AR¹, AR², AR³, AR⁴, AR⁵ and AR⁶ are simultaneously hydrogen.

In another embodiment of the present invention, AR² is X-AR^{X}, wherein X is —SO₂NAR′—, and AR^{X} is AR′; i.e., AR² is —SO₂N(AR′)₂. In one embodiment, the two AR′ therein taken together form an optionally substituted 5-7 membered ring with 0-3 additional heteroatoms selected from nitrogen, oxygen, or sulfur. Or, AR¹, AR³, AR⁴, AR⁵ and AR⁶ are simultaneously hydrogen, and AR² is SO₂N(AR′)₂.

In some embodiments, X is a bond or is an optionally substituted C₁₋₆ alkylidene chain wherein one or two non-adjacent methylene units are optionally and independently replaced by O, NAR′, S, SO₂, or COO, CO, and AR^{X} is AR′ or halo. In still other embodiments, each occurrence of XAR^{X} is independently —C₁₋₃alkyl, —O(C₁₋₃alkyl), —CF₃, —OCF₃, —SCF₃, —F, —Cl, —Br, OH, —COOAR′, —COAR′, —O(CH₂)₂N(AR′)(AR′), —O(CH₂)N(AR′)(AR′), —CON(AR′)(AR′), —(CH₂)₂OAR′, —(CH₂)OAR′, optionally substituted phenyl, —N(AR′)(AR′), —(CH₂)₂N(AR′)(AR′), or —(CH₂)N(AR′)(AR′).

In some embodiments, AR⁷ is hydrogen. In certain other embodiment, AR⁷ is C₁₋₄ straight or branched aliphatic.

In some embodiments, AR^{W} is selected from halo, cyano, CF₃, CHF₂, OCHF₂, Me, Et, CH(Me)₂, CHMeEt, n-propyl, t-butyl, OMe, OEt, OPh, O-fluorophenyl, O-difluorophenyl, O-methoxyphenyl, O-tolyl, O-benzyl, SMe, SCF₃, SCHF₂, SEt, CH₂CN, NH₂, NHMe, N(Me)₂, NHEt, N(Et)₂, C(O)CH₃, C(O)Ph, C(O)NH₂, SPh, SO₂— (amino-pyridyl), SO₂NH₂, SO₂Ph, SO₂NHPh, SO₂—N-morpholino, SO₂—N-pyrrolidyl, N-pyrrolyl, N-morpholino, 1-piperidyl, phenyl, benzyl, (cyclohexyl-methylamino)methyl, 4-Methyl-2,4-dihydro-pyrazol-3-one-2-yl, benzimidazol-2y1, furan-2-yl, 4-methyl-4H-[1,2,4]triazol-3-yl, 3-(4′-chlorophenyl)-[1,2,4]oxadiazol-5-yl, NHC(O)Me, NHC(O)Et, NHC(O)Ph, NHSO₂Me, 2-indolyl, 5-indolyl, —CH₂CH₂OH, —OCF₃, O-(2,3-dimethylphenyl), 5-methylfuryl, —SO₂—N-piperidyl, 2-tolyl, 3-tolyl, 4-tolyl, O-butyl, NHCO₂C(Me)₃, CO₂C(Me)₃, isopropenyl, n-butyl, O-(2,4-dichlorophenyl), NHSO₂PhMe, O-(3-chloro-5-trifluoromethyl-2-pyridyl), phenylhydroxymethyl, 2,5-dimethylpyrrolyl, NHCOCH₂C(Me)₃, O-(2-tert-butyl)phenyl, 2,3-dimethylphenyl, 3,4-dimethylphenyl, 4-hydroxymethyl phenyl, 4-dimethylaminophenyl, 2-trifluoromethylphenyl, 3-trifluoromethylphenyl, 4-trifluoromethylphenyl, 4-cyanomethylphenyl, 4-isobutylphenyl, 3-pyridyl, 4-pyridyl, 4-isopropylphenyl, 3-isopropylphenyl, 2-methoxyphenyl, 3-methoxyphenyl, 4-methoxyphenyl, 3,4-methylenedioxyphenyl, 2-ethoxyphenyl, 3-ethoxyphenyl, 4-ethoxyphenyl, 2-methylthiophenyl, 4-methylthiophenyl, 2,4-dimethoxyphenyl, 2,5-dimethoxyphenyl, 2,6-dimethoxyphenyl, 3,4-dimethoxyphenyl, 5-chloro-2-methoxyphenyl, 2-OCF₃-phenyl, 3-trifluoromethoxy-phenyl, 4-trifluoromethoxyphenyl, 2-phenoxyphenyl, 4-phenoxyphenyl, 2-fluoro-3-methoxy-phenyl, 2,4-dimethoxy-5-pyrimidyl, 5-isopropyl-2-methoxyphenyl, 2-fluorophenyl, 3-fluorophenyl, 4-fluorophenyl, 3-cyanophenyl, 3-chlorophenyl, 4-chlorophenyl, 2,3-difluorophenyl, 2,4-difluorophenyl, 2,5-difluorophenyl, 3,4-difluorophenyl, 3,5-difluorophenyl, 3-chloro-4-fluoro-phenyl, 3,5-dichlorophenyl, 2,5-dichlorophenyl, 2,3-dichlorophenyl, 3,4-dichlorophenyl, 2,4-dichlorophenyl, 3-methoxycarbonylphenyl, 4-methoxycarbonyl phenyl, 3-isopropyloxycarbonylphenyl, 3-acetamidophenyl, 4-fluoro-3-methylphenyl, 4-methanesulfinyl-phenyl, 4-methanesulfonyl-phenyl, 4-N-(2-N,N-dimethylaminoethyl)carbamoylphenyl, 5-acetyl-2-thienyl, 2-benzothienyl, 3-benzothienyl, furan-3-yl, 4-methyl-2-thienyl, 5-cyano-2-thienyl, N′-phenylcarbonyl-N-piperazinyl, —NHCO₂Et, —NHCO₂Me, N-pyrrolidinyl, —NHSO₂(CH₂)₂ N-piperidine, —NHSO₂(CH₂)₂ N-morpholine, —NHSO₂(CH₂)₂N(Me)₂, COCH₂N(Me)COCH₂NHMe, —CO₂Et, O-propyl, —CH₂CH₂NHCO₂C(Me)₃, hydroxy, aminomethyl, pentyl, adamantyl, cyclopentyl, ethoxyethyl, C(Me)₂CH₂OH, C(Me)₂CO₂Et, —CHOHMe, CH₂CO₂Et, —C(Me)₂CH₂NHCO₂C(Me)₃, O(CH₂)₂OEt, O(CH₂)₂OH, CO₂Me, hydroxymethyl, 1-methyl-1-cyclohexyl, 1-methyl-1-cyclooctyl, 1-methyl-1-cycloheptyl, C(Et)₂C(Me)₃, C(Et)₃, CONHCH₂CH(Me)₂, 2-aminomethyl-phenyl, ethenyl, 1-piperidinylcarbonyl, ethynyl, cyclohexyl, 4-methylpiperidinyl, —OCO₂Me, —C(Me)₂CH₂NHCO₂CH₂CH(Me)₂, —C(Me)₂CH₂NHCO₂CH₂CH₂CH₃, —C(Me)₂CH₂NHCO₂Et, —C(Me)₂CH₂NHCO₂Me, —C(Me)₂CH₂NHCO₂CH₂C(Me)₃, —CH₂NHCOCF₃, —CH₂NHCO₂C(Me)₃, —C(Me)₂CH₂NHCO₂(CH₂)₃CH₃, C(Me)₂CH₂NHCO₂(CH₂)₂OMe, C(OH)(CF₃)₂, —C(Me)₂CH₂NHCO₂CH₂-tetrahydrofurane-3-yl, C(Me)₂CH₂O(CH₂)₂OMe, or 3-ethyl-2,6-dioxopiperidin-3-yl.

In one embodiment, AR′ is hydrogen.

In one embodiment, AR′ is a C1-C8 aliphatic group, optionally substituted with up to 3 substituents selected from halo, CN, CF₃, CHF₂, OCF₃, or OCHF₂, wherein up to two methylene units of said C1-C8 aliphatic is optionally replaced with —CO—, —CONH(C1-C4 alkyl)-, —CO₂—, —OCO—, —N(C1-C4 alkyl)CO₂—, —O—, —N(C1-C4 alkyl)CON(C1-C4 alkyl)-, —OCON(C1-C4 alkyl)-, —N(C1-C4 alkyl)CO—, —S—, —N(C1-C4 alkyl)-, —SO₂N(C1-C4 alkyl)-, N(C1-C4 alkyl)SO₂—, or —N(C1-C4 alkyl)SO₂N(C1-C4 alkyl)-.

In one embodiment, AR′ is a 3-8 membered saturated, partially unsaturated, or fully unsaturated monocyclic ring having 0-3 heteroatoms independently selected from nitrogen, oxygen, or sulfur, wherein AR′ is optionally substituted with up to 3 substituents selected from halo, CN, CF₃, CHF₂, OCF₃, OCHF₂, or C1-C6 alkyl, wherein up to two methylene units of said C1-C6 alkyl is optionally replaced with —CO—, —CONH(C1-C4 alkyl)-, —CO₂—, —OCO—, —N(C1-C4 alkyl)CO₂—, —O—, —N(C1-C4 alkyl)CON(C1-C4 alkyl)-, —OCON(C1-C4 alkyl)-, —N(C1-C4 alkyl)CO—, —S—, —N(C1-C4 alkyl)-, —SO₂N(C1-C4 alkyl)-, N(C1-C4 alkyl)SO₂—, or —N(C1-C4 alkyl)SO₂N(C1-C4 alkyl)-.

In one embodiment, AR′ is an 8-12 membered saturated, partially unsaturated, or fully unsaturated bicyclic ring system having 0-5 heteroatoms independently selected from nitrogen, oxygen, or sulfur; wherein AR′ is optionally substituted with up to 3 substituents selected from halo, CN, CF₃, CHF₂, OCF₃, OCHF₂, or C1-C6 alkyl, wherein up to two methylene units of said C1-C6 alkyl is optionally replaced with —CO—, —CONH(C1-C4 alkyl)-, —CO₂—, —OCO—, —N(C1-C4 alkyl)CO₂—, —O—, —N(C1-C4 alkyl)CON(C1-C4 alkyl)-, —OCON(C1-C4 alkyl)-, —N(C1-C4 alkyl)CO—, —S—, —N(C1-C4 alkyl)-, —SO₂N(C1-C4 alkyl)-, N(C1-C4 alkyl)SO₂—, or —N(C1-C4 alkyl)SO₂N(C1-C4 alkyl)-.

In one embodiment, two occurrences of AR′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur, wherein AR′ is optionally substituted with up to 3 substituents selected from halo, CN, CF₃, CHF₂, OCF₃, OCHF₂, or C1-C6 alkyl, wherein up to two methylene units of said C1-C6 alkyl is optionally replaced with —CO—, —CONH(C1-C4 alkyl)-, —CO₂—, —OCO—, —N(C1-C4 alkyl)CO₂—, —O—, —N(C1-C4 alkyl)CON(C1-C4 alkyl)-, —OCON(C1-C4 alkyl)-, —N(C1-C4 alkyl)CO—, —S—, —N(C1-C4 alkyl)-, —SO₂N(C1-C4 alkyl)-, N(C1-C4 alkyl)SO₂—, or —N(C1-C4 alkyl)SO₂N(C1-C4 alkyl)-.

According to one embodiment, the present invention provides compounds of formula AIIA or formula AIIB:

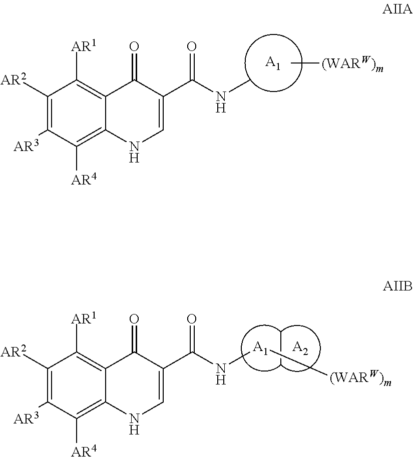

According to another embodiment, the present invention provides compounds of formula AIIIA, formula AIIIB, formula AIIIC, formula AIIID, or formula AIIIE:

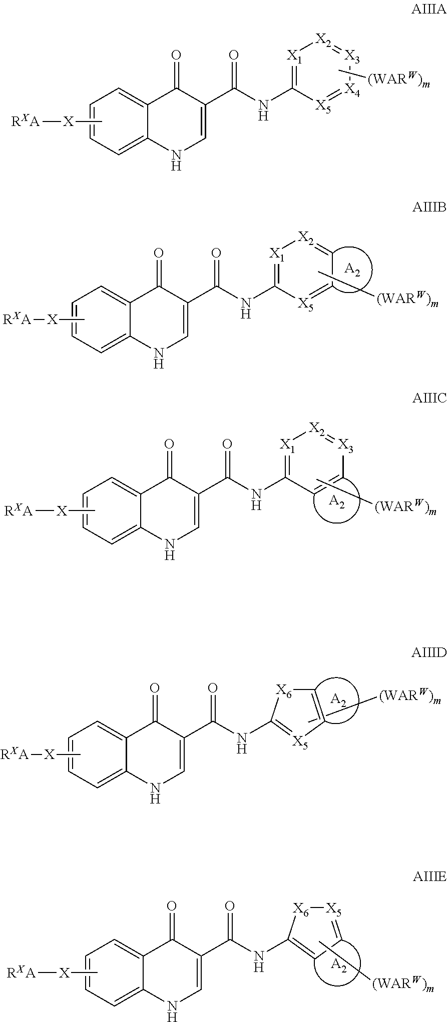

wherein each of X₁, X₂, X₃, X₄, and X₅ is independently selected from CH or N; and X₆ is O, S, or NAR′.

In one embodiment, compounds of formula AIIIA, formula AIIIB, formula AIIIC, formula AIIID, or formula AIIIE have y occurrences of substituent X-AR^{X}, wherein y is 0-4. Or, y is 1. Or, y is 2.

In some embodiments of formula AIIIA, X₁, X₂, X₃, X₄, and X₅ taken together with WAR^{W} and m is optionally substituted phenyl.

In some embodiments of formula ARIA, X₁, X₂, X₃, X₄, and X₅ taken together is an optionally substituted ring selected from:

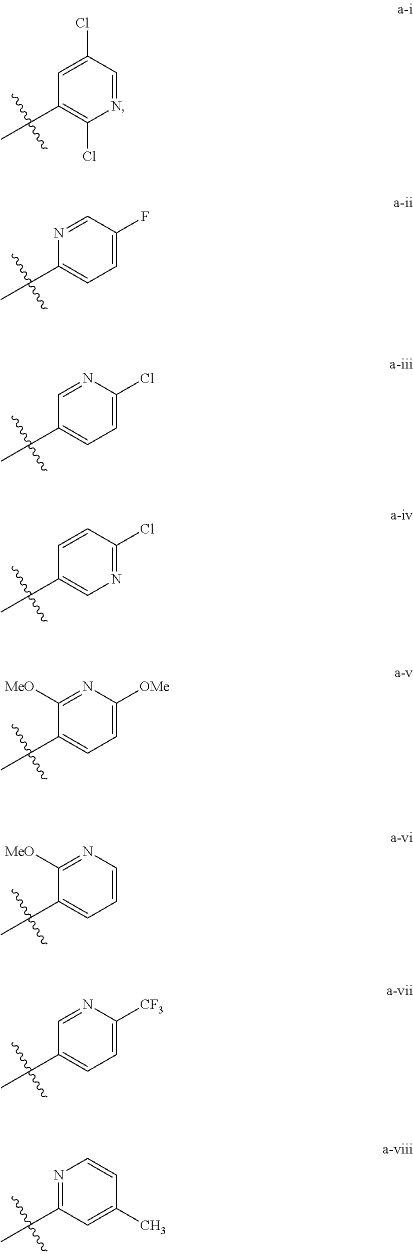

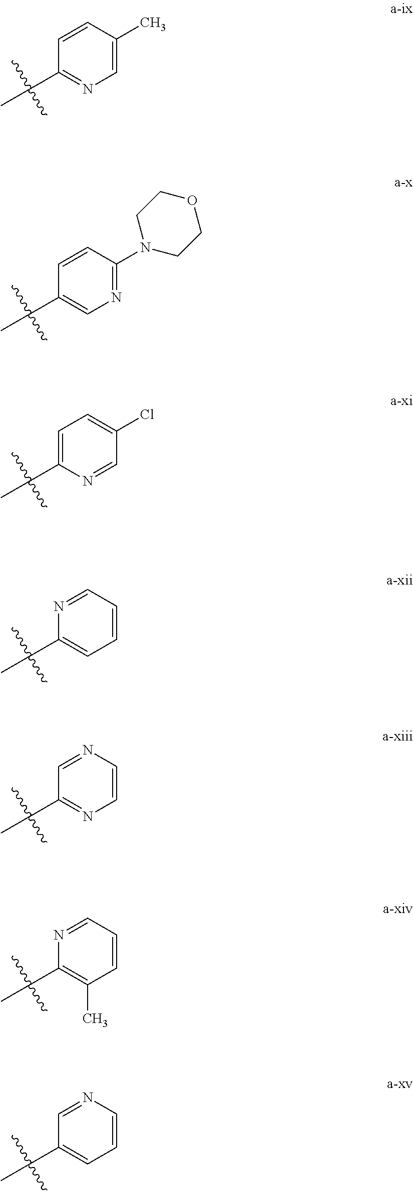

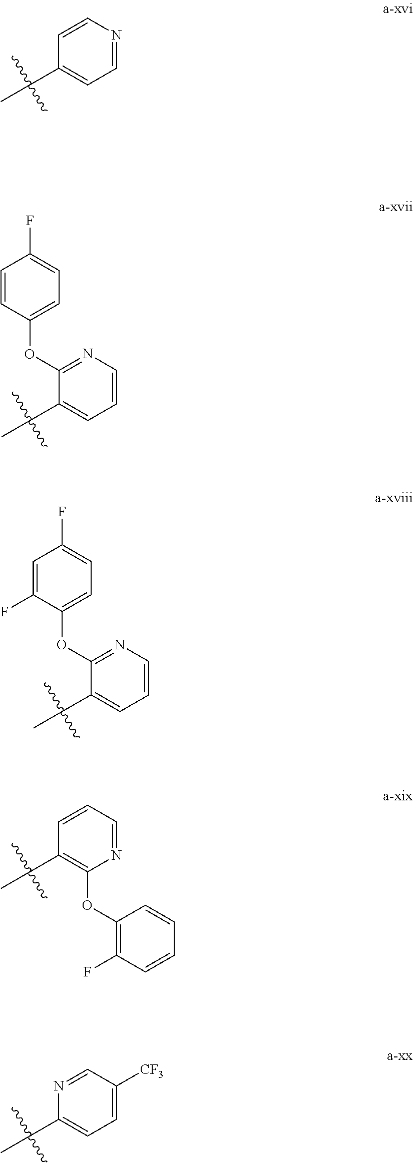 [SMILES: *c1ccncc1 ; *c1cccnc1Oc1ccc(F)cc1 ; *c1cccnc1Oc1ccc(F)cc1F ; *c1cccnc1Oc1ccccc1F ; *c1ccc(C(F)(F)F)cn1]

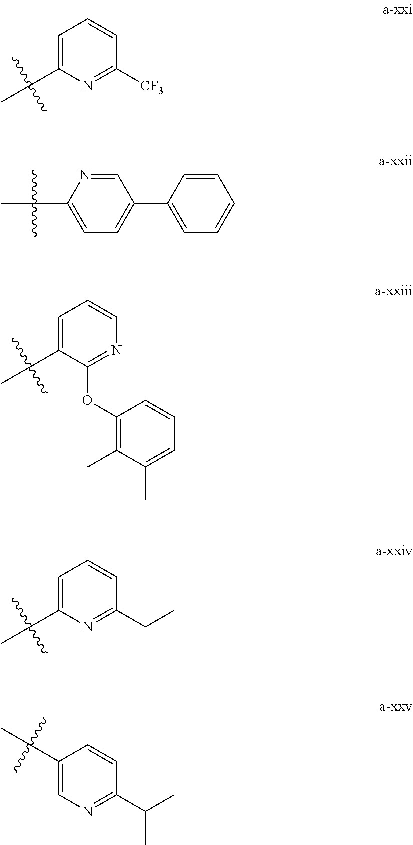

In some embodiments of formula AIIIB, formula AIIIC, formula AIIID, or formula AIIIE, X₁, X₂, X₃, X₄, X₅, or X₆, taken together with ring A₂ is an optionally substituted ring selected from:

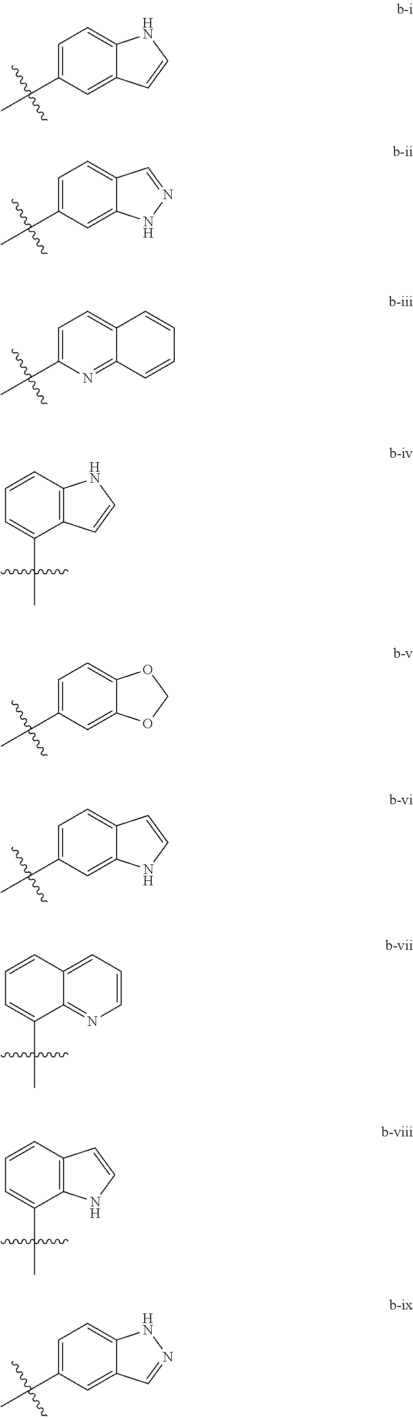

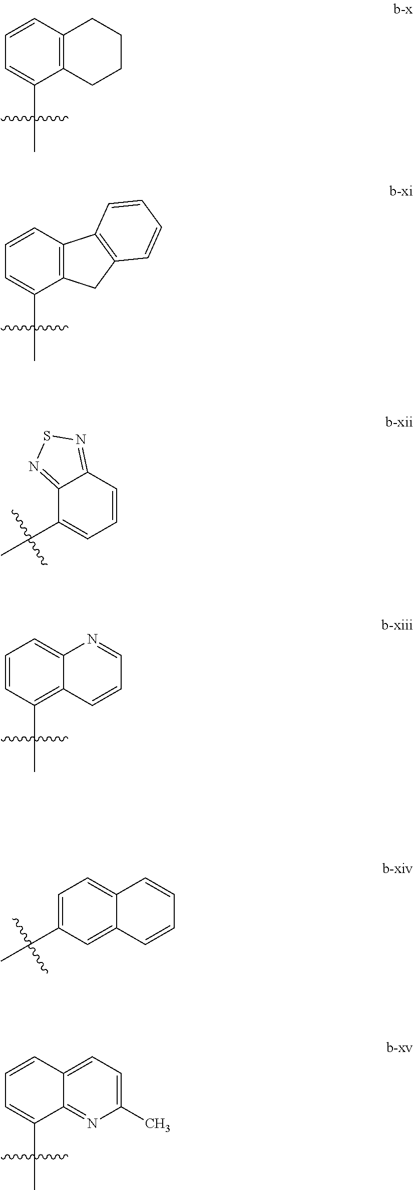 [SMILES: *c1cccc2c1CCCC2 ; *c1cccc2c1Cc1ccccc1-2 ; *c1cccc2nsnc12 ; *c1cccc2ncccc12 ; *c1ccc2ccccc2c1 ; *c1cccc2ccc(C)nc12]

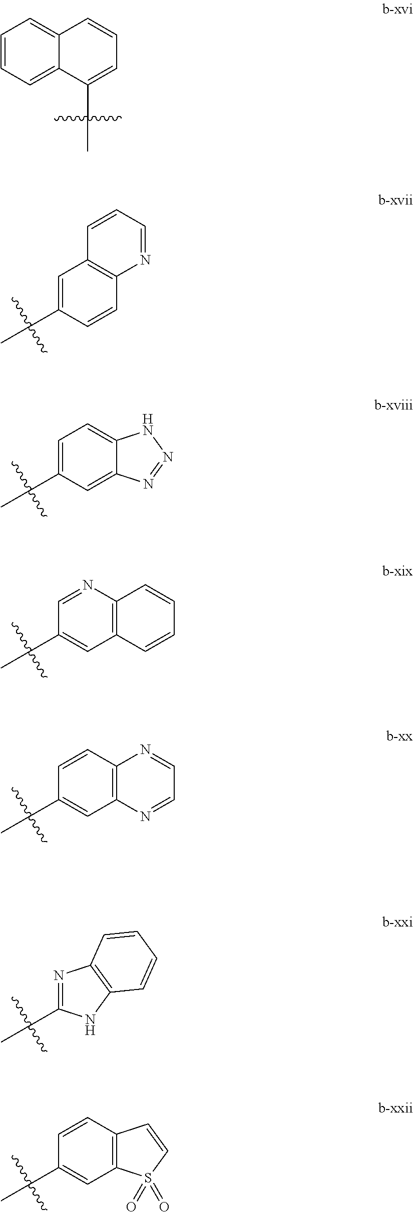

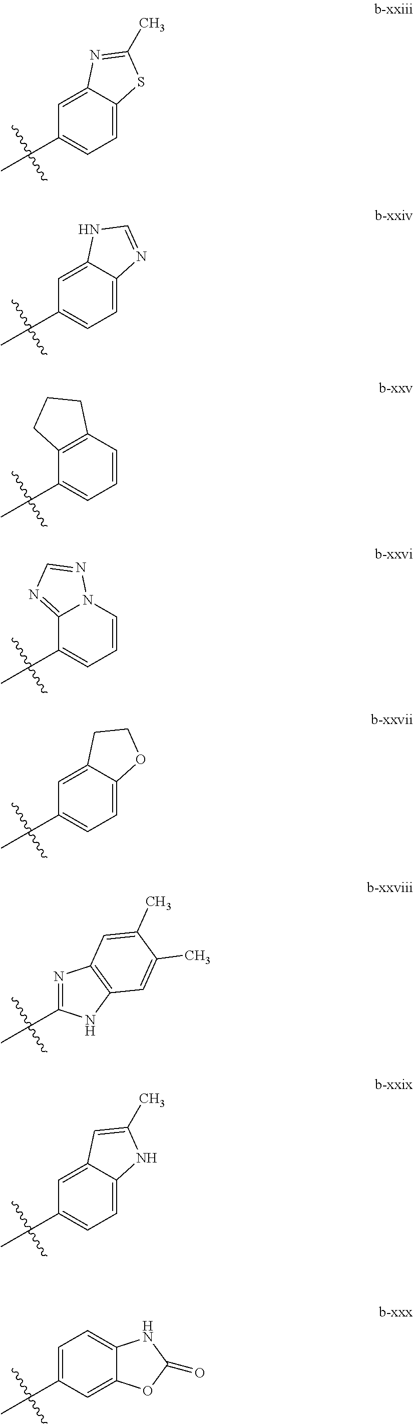

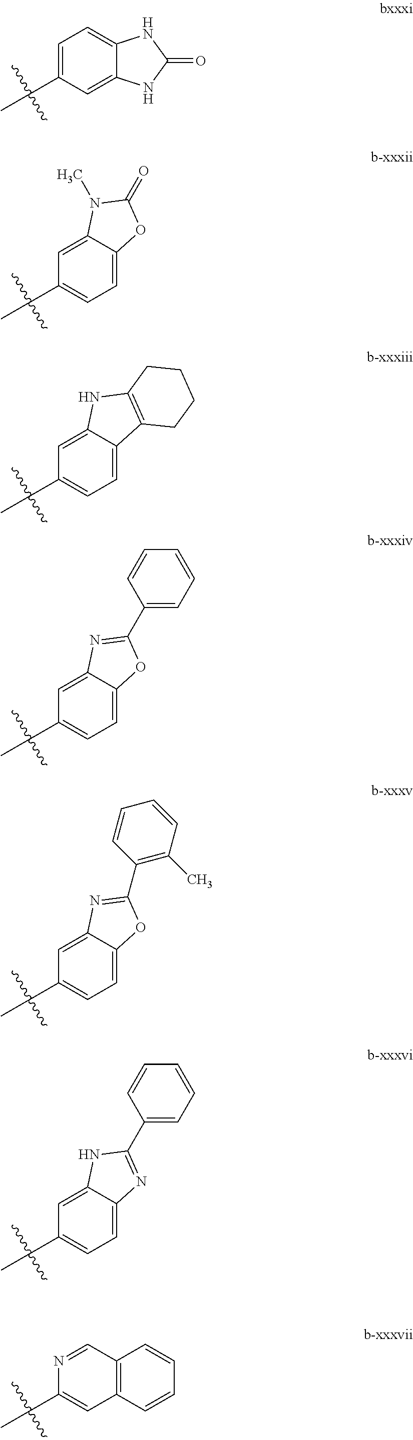

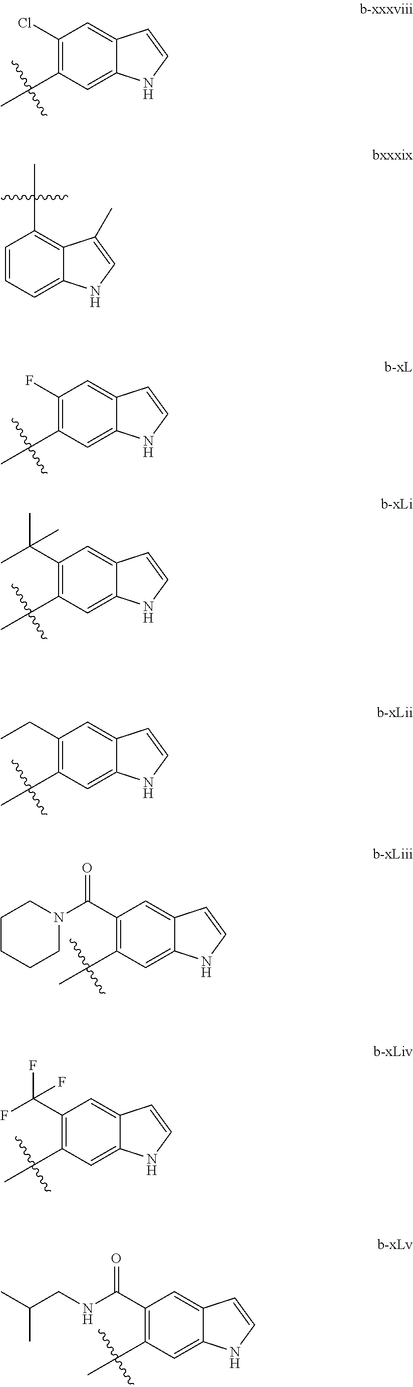

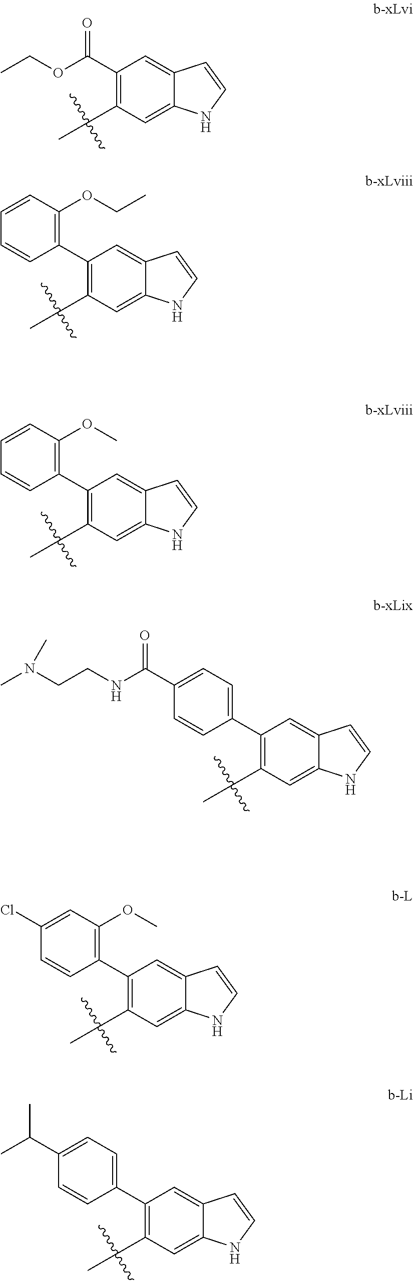 [SMILES: *c1cc2[nH]ccc2cc1C(=O)OCC ; *c1cc2[nH]ccc2cc1-c1ccccc1OCC ; *c1cc2[nH]ccc2cc1-c1ccccc1OC ; *c1cc2[nH]ccc2cc1-c1ccc(C(=O)NCCN(C)C)cc1 ; *c1cc2[nH]ccc2cc1-c1ccc(Cl)cc1OC ; *c1cc2[nH]ccc2cc1-c1ccc(C(C)C)cc1]

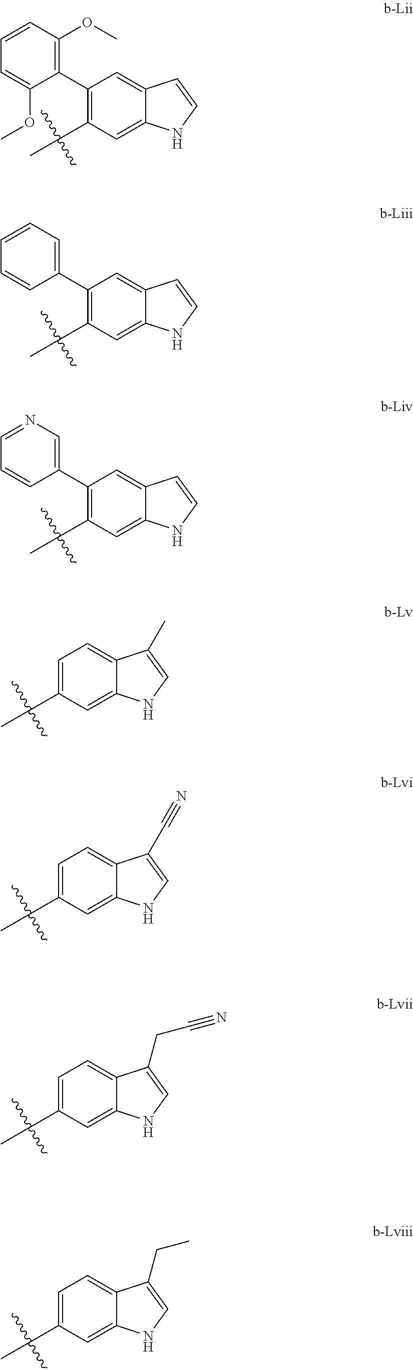

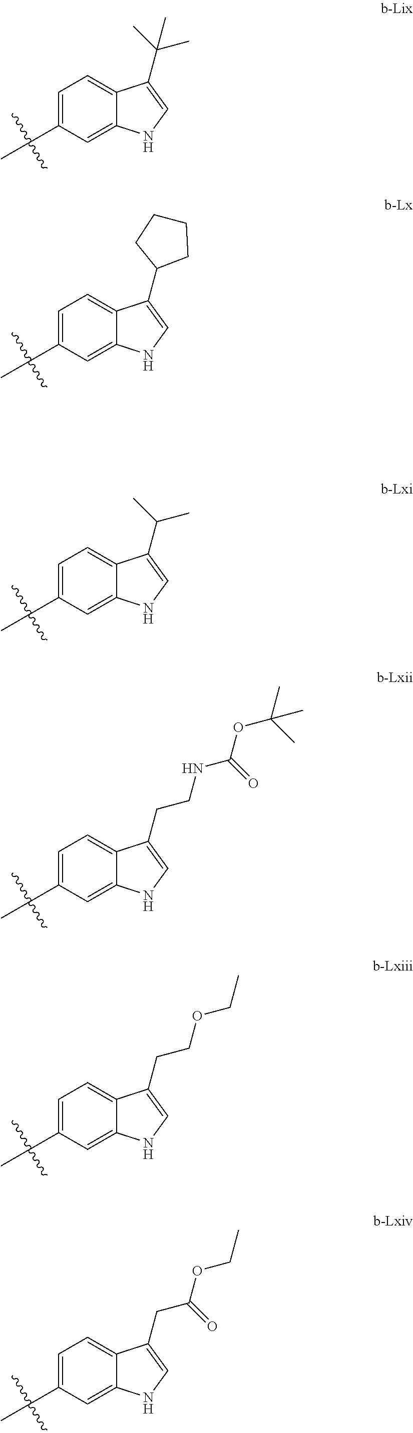 [SMILES: *c1ccc2c(C(C)(C)C)c[nH]c2c1 ; *c1ccc2c(C3CCCC3)c[nH]c2c1 ; *c1ccc2c(C(C)C)c[nH]c2c1 ; *c1ccc2c(CCNC(=O)OC(C)(C)C)c[nH]c2c1 ; *c1ccc2c(CCOCC)c[nH]c2c1 ; *c1ccc2c(CC(=O)OCC)c[nH]c2c1]

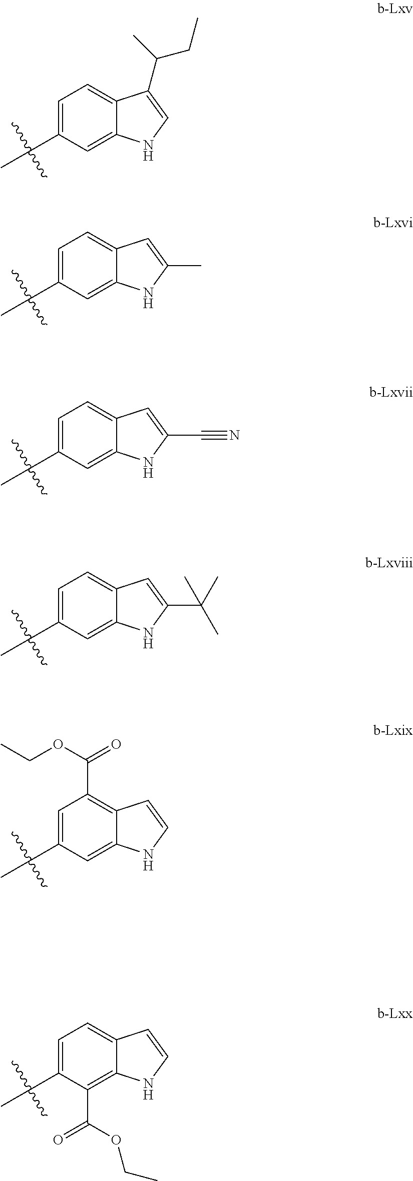

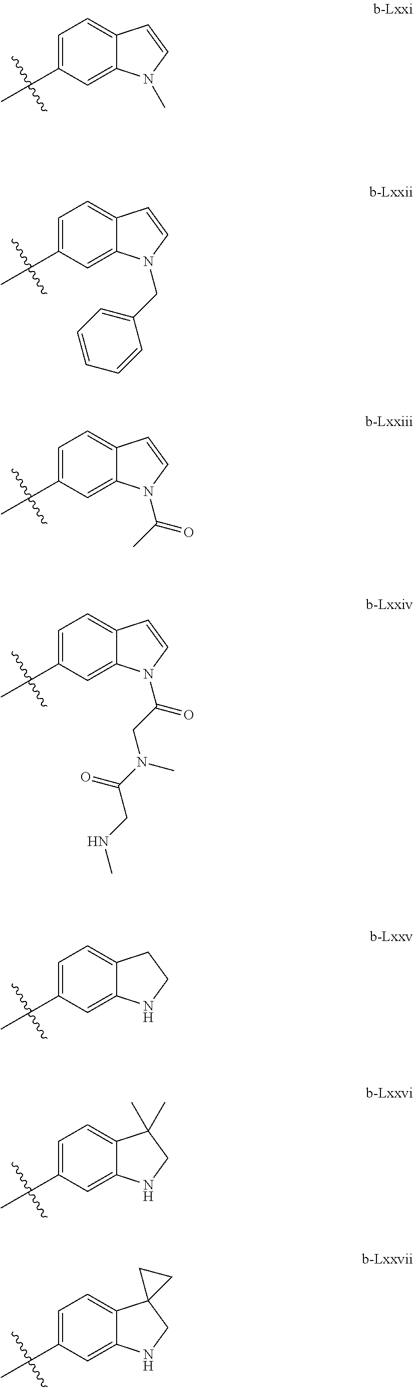

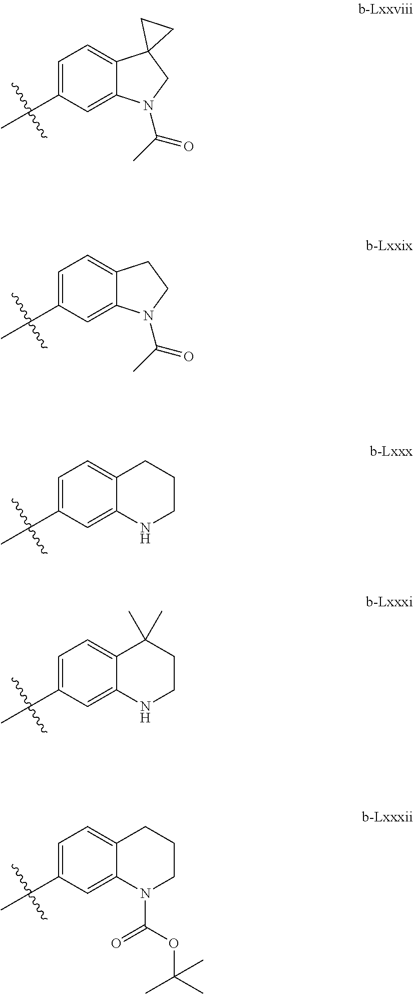 [SMILES: *c1ccc2c(c1)N(C(C)=O)CC21CC1 ; *c1ccc2c(c1)N(C(C)=O)CC2 ; *c1ccc2c(c1)NCCC2 ; *c1ccc2c(c1)NCCC2(C)C ; *c1ccc2c(c1)N(C(=O)OC(C)(C)C)CCC2]

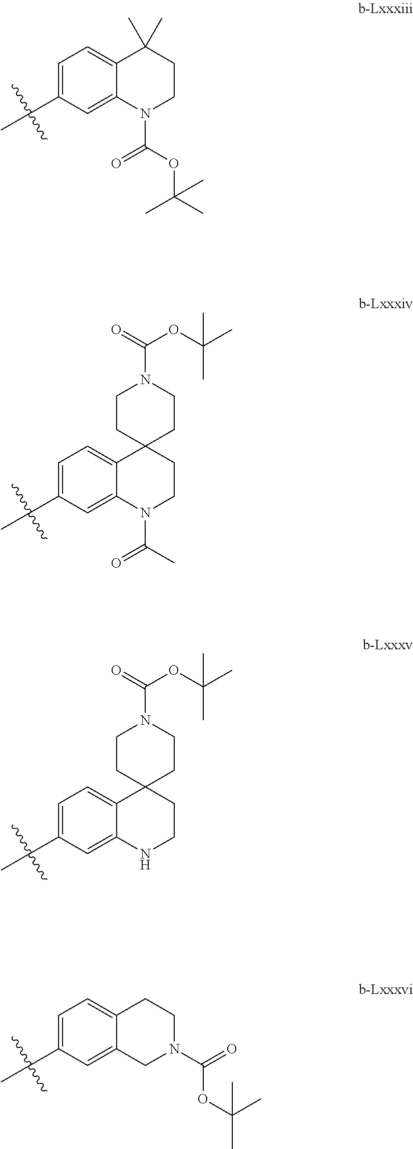 [SMILES: *c1ccc2c(c1)N(C(=O)OC(C)(C)C)CCC2(C)C ; *c1ccc2c(c1)N(C(C)=O)CCC21CCN(C(=O)OC(C)(C)C)CC1 ; *c1ccc2c(c1)NCCC21CCN(C(=O)OC(C)(C)C)CC1 ; *c1ccc2c(c1)CN(C(=O)OC(C)(C)C)CC2]

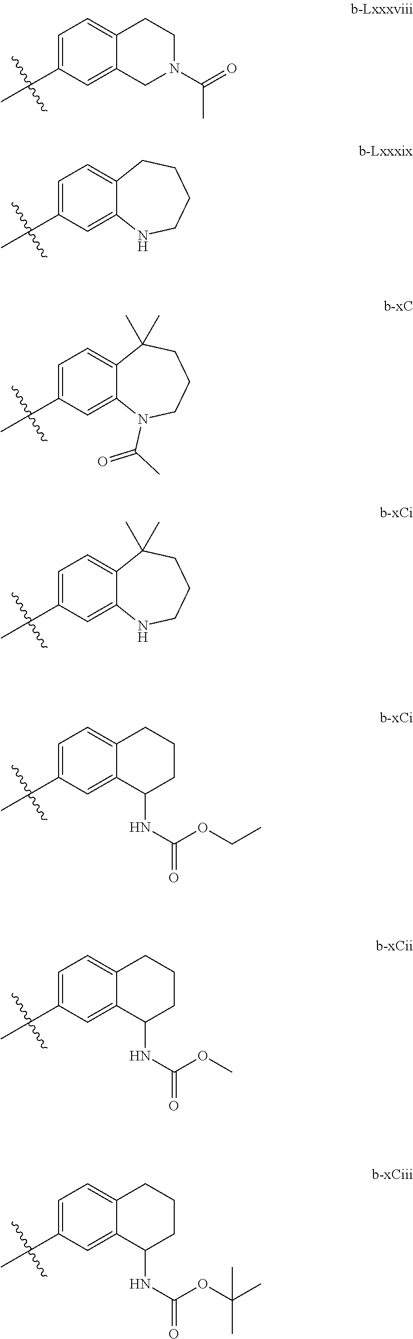

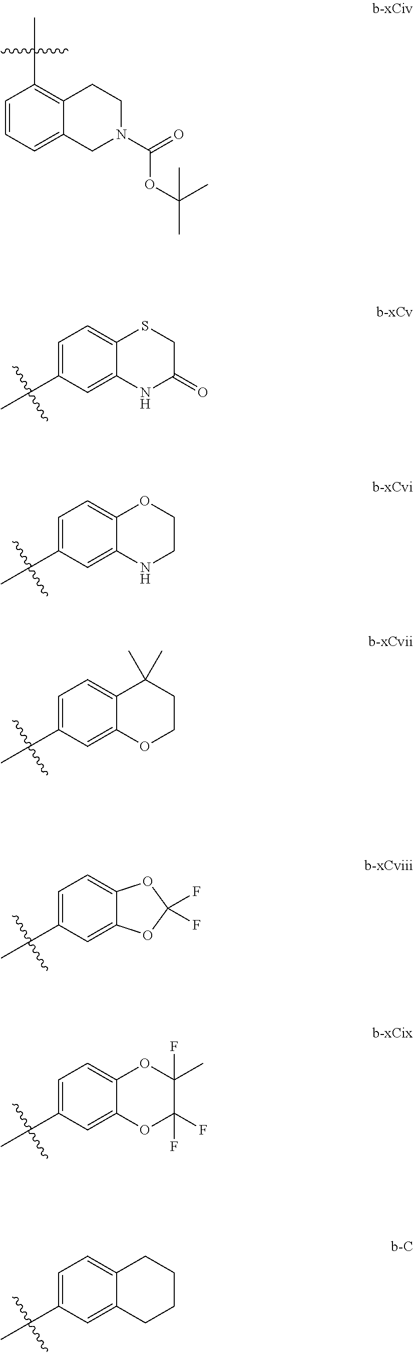

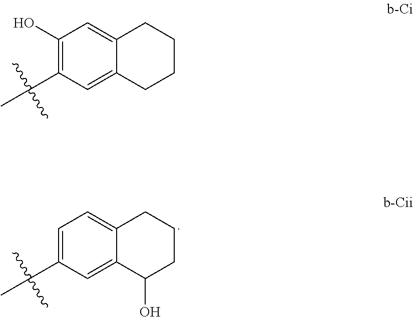 [SMILES: *c1cc2c(cc1O)CCCC2 ; *c1ccc2c(c1)C(O)CCC2]

In some embodiments, AR^{W} is selected from halo, cyano, CF₃, CHF₂, OCHF₂, Me, Et, CH(Me)₂, CHMeEt, n-propyl, t-butyl, OMe, OEt, OPh, O-fluorophenyl, O-difluorophenyl, O-methoxyphenyl, O-tolyl, O-benzyl, SMe, SCF₃, SCHF₂, SEt, CH₂CN, NH₂, NHMe, N(Me)₂, NHEt, N(Et)₂, C(O)CH₃, C(O)Ph, C(O)NH₂, SPh, SO₂-(amino-pyridyl), SO₂NH₂, SO₂Ph, SO₂NHPh, SO₂—N-morpholino, SO₂—N-pyrrolidyl, N-pyrrolyl, N-morpholino, 1-piperidyl, phenyl, benzyl, (cyclohexyl-methylamino)methyl, 4-Methyl-2,4-dihydro-pyrazol-3-one-2-yl, benzimidazol-2yl, furan-2-yl, 4-methyl-4H-[1,2,4]triazol-3-yl, 3-(4′-chlorophenyl)-[1,2,4]oxadiazol-5-yl, NHC(O)Me, NHC(O)Et, NHC(O)Ph, or NHSO₂Me

In some embodiments, X and AR^{X}, taken together, is Me, Et, halo, CN, CF₃, OH, OMe, OEt, SO₂N(Me)(fluorophenyl), SO₂-(4-methyl-piperidin-1-yl, or SO₂—N-pyrrolidinyl.

According to another embodiment, the present invention provides compounds of formula AIVA, formula AIVB, or formula AIVC:

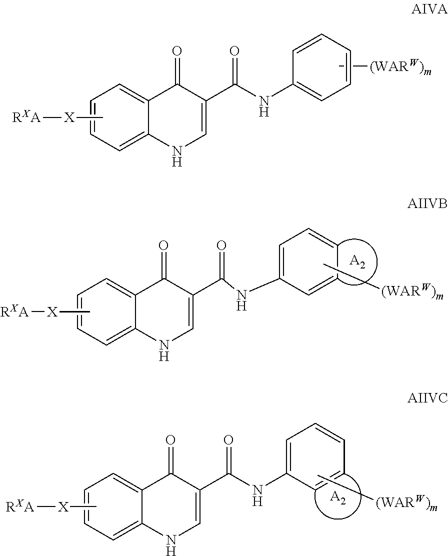

In one embodiment compounds of formula AIVA, formula AIVB, and formula AIVC have y occurrences of substituent X-AR^{X}, wherein y is 0-4. Or, y is 1. Or, y is 2.

In one embodiment, the present invention provides compounds of formula AIVA, formula AIVB, and formula AIVC, wherein X is a bond and AR^{X} is hydrogen.

In one embodiment, the present invention provides compounds of formula formula AIVB, and formula AIVC, wherein ring A₂ is an optionally substituted, saturated, unsaturated, or aromatic seven membered ring with 0-3 heteroatoms selected from O, S, or N. Exemplary rings include azepanyl, 5,5-dimethyl azepanyl, etc.

In one embodiment, the present invention provides compounds of formula AIVB and AIVC, wherein ring A₂ is an optionally substituted, saturated, unsaturated, or aromatic six membered ring with 0-3 heteroatoms selected from O, S, or N. Exemplary rings include piperidinyl, 4,4-dimethylpiperidinyl, etc.

In one embodiment, the present invention provides compounds of formula AIVB and AIVC, wherein ring A₂ is an optionally substituted, saturated, unsaturated, or aromatic five membered ring with 0-3 heteroatoms selected from O, S, or N.

In one embodiment, the present invention provides compounds of formula IVB and IVC, wherein ring A₂ is an optionally substituted five membered ring with one nitrogen atom, e.g., pyrrolyl or pyrrolidinyl.

According to one embodiment of formula AIVA, the following compound of formula AVA-1 is provided:

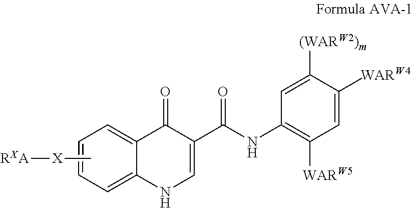

wherein each of WAR^{W2} and WAR^{W4} is independently selected from hydrogen, CN, CF₃, halo, C1-C6 straight or branched alkyl, 3-12 membered cycloaliphatic, phenyl, C5-C10 heteroaryl or C3-C7 heterocyclic, wherein said heteroaryl or heterocyclic has up to 3 heteroatoms selected from O, S, or N, wherein said WAR^{W2} and WAR^{W4} is independently and optionally substituted with up to three substituents selected from —OAR′, —CF₃, —OCF₃, SR′, S(O)AR′, SO₂AR′, —SCF₃, halo, CN, —COOAR′, —COAR′, —O(CH₂)₂N(AR′)(AR′), —O(CH₂)N(AR′)(AR′), —CON(AR′)(AR′), —(CH₂)₂OAR′, —(CH₂)OAR′, CH₂CN, optionally substituted phenyl or phenoxy, —N(AR′)(AR′), —NAR′C(O)OAR′, —NAR′C(O)AR′, —(CH₂)₂N(AR′)(AR′), or —(CH₂)N(AR′)(AR′); and

WAR^{W5} is selected from hydrogen, —OH, NH₂, CN, CHF₂, NHR′, N(AR′)₂, —NHC(O)AR′, —NHC(O)OAR′, NHSO₂AR′, —OAR′, CH₂OH, CH2N(AR′)₂, C(O)OAR′, SO₂NHAR′, SO₂N(AR′)₂, or CH₂NHC(O)OAR′. Or, WAR^{W4} and WAR^{W5} taken together form a 5-7 membered ring containing 0-3 three heteroatoms selected from N, O, or S, wherein said ring is optionally substituted with up to three WAR^{W} substituents.

In one embodiment, compounds of formula AVA-1 have y occurrences of X-AR^{X}, wherein y is 0-4. In one embodiment, y is 0.

In one embodiment, the present invention provides compounds of formula AVA-1, wherein X is a bond and AR^{X} is hydrogen.

In one embodiment, the present invention provides compounds of formula AVA-1, wherein:

each of WAR^{W2} and WAR^{W4} is independently selected from hydrogen, CN, CF₃, halo, C1-C6 straight or branched alkyl, 3-12 membered cycloaliphatic, or phenyl, wherein said WAR^{W2} and WAR^{W4} is independently and optionally substituted with up to three substituents selected from —OAR′, —CF₃, —OCF₃, —SCF₃, halo, —COOAR′, —COAR′, —O(CH₂)₂N(AR′)(AR′), —O(CH₂)N(AR′)(AR′), —CON(AR′)(AR′), —(CH₂)₂OAR′, —(CH₂)OAR′, optionally substituted phenyl, —N(AR′)(AR′), —NC(O)OAR′, —NC(O)AR′, —(CH₂)₂N(AR′)(AR′), or —(CH₂)N(AR′)(AR′); and

WAR^{W5} is selected from hydrogen, —OH, NH₂, CN, NHAR′, N(AR′)₂, —NHC(O)AR′, —NHC(O)OAR′, NHSO₂AR′, —OAR′, CH₂OH, C(O)OAR′, SO₂NHAR′, or CH₂NHC(O)O-(AR′).

In one embodiment, the present invention provides compounds of formula AVA-1, wherein:

WAR^{W2} is a phenyl ring optionally substituted with up to three substituents selected from —OAR′, —CF₃, —OCF₃, SAR′, S(O)AR′, SO₂AR′, —SCF₃, halo, CN, —COOAR′, —COAR′, —O(CH₂)₂N(AR′)(AR′), —O(CH₂)N(AR′)(AR′), —CON(AR′)(AR′), —(CH₂)₂OAR′, —(CH₂)OAR′, CH₂CN, optionally substituted phenyl or phenoxy, —N(AR′)(AR′), —NAR′C(O)OAR′, —NAR′C(O)AR′, —(CH₂)₂N(AR′)(AR′), or —(CH₂)N(AR′)(AR′);

WAR^{W4} is C1-C6 straight or branched alkyl; and

WAR^{W5} is OH.

In one embodiment, each of WAR^{W2} and WAR^{W4} is independently selected from CF₃ or halo. In one embodiment, each of WAR^{W2} and WAR^{W4} is independently selected from optionally substituted hydrogen, C1-C6 straight or branched alkyl. In certain embodiments, each of WAR^{W2} and WAR^{W4} is independently selected from optionally substituted n-propyl, isopropyl, n-butyl, sec-butyl, t-butyl, 1,1-dimethyl-2-hydroxyethyl, 1,1-dimethyl-2-(ethoxycarbonyl)-ethyl, 1,1-dimethyl-3-(t-butoxycarbonyl-amino) propyl, or n-pentyl.

In one embodiment, each of WAR^{W2} and WAR^{W4} is independently selected from optionally substituted 3-12 membered cycloaliphatic. Exemplary embodiments of such cycloaliphatic include cyclopentyl, cyclohexyl, cycloheptyl, norbornyl, adamantyl, [2.2.2.]bicyclo-octyl, [2.3.1.]bicyclo-octyl, or [3.3.1]bicyclo-nonyl.

In certain embodiments WAR^{W2} is hydrogen and WAR^{W4} is C1-C6 straight or branched alkyl. In certain embodiments, WAR^{W4} is selected from methyl, ethyl, propyl, n-butyl, sec-butyl, or t-butyl.

In certain embodiments WAR^{W4} is hydrogen and WAR^{W2} is C1-C6 straight or branched alkyl. In certain embodiments, WAR^{W2} is selected from methyl, ethyl, propyl, n-butyl, sec-butyl, t-butyl, or n-pentyl.

In certain embodiments each of WAR^{W2} and WAR^{W4} is C1-C6 straight or branched alkyl. In certain embodiments, each of WAR^{W2} and WAR^{W4} is selected from methyl, ethyl, propyl, n-butyl, sec-butyl, t-butyl, or pentyl.

In one embodiment, WAR^{W5} is selected from hydrogen, CHF₂, NH₂, CN, NHR′, N(AR′)₂, CH₂N(AR′)₂, —NHC(O)AR′, —NHC(O)OAR′, —OAR′, C(O)OAR′, or SO₂NHAR′. Or, WAR^{W5} is —OAR′, e.g., OH.

In certain embodiments, WAR^{W5} is selected from hydrogen, NH₂, CN, CHF₂, NH(C1-C6 alkyl), N(C1-C6 alkyl)₂, —NHC(O)(C1-C6 alkyl), —CH₂NHC(O)O(C1-C6 alkyl), —NHC(O)O(C1-C6 alkyl), —OH, —O(C1-C6 alkyl), C(O)O(C1-C6 alkyl), CH₂O(C1-C6 alkyl), or SO₂NH₂. In another embodiment, WAR^{W5} is selected from —OH, OMe, NH₂, —NHMe, —N(Me)₂, —CH₂NH₂, CH₂OH, NHC(O)OMe, NHC(O)OEt, CN, CHF₂, —CH₂NHC(O)O(t-butyl), —O-(ethoxyethyl), —O-(hydroxyethyl), —C(O)OMe, or —SO₂NH₂.

In one embodiment, compound of formula AVA-1 has one, preferably more, or more preferably all, of the following features:

WAR^{W2} is hydrogen;

WAR^{W4} is C1-C6 straight or branched alkyl or monocyclic or bicyclic aliphatic; and

WAR^{W5} is selected from hydrogen, CN, CHF₂, NH₂, NH(C1-C6 alkyl), N(C1-C6 alkyl)₂, —NHC(O)(C1-C6 alkyl), —NHC(O)O(C1-C6 alkyl), —CH₂C(O)O(C1-C6 alkyl), —OH, —O(C1-C6 alkyl), C(O)O(C1-C6 alkyl), or SO₂NH₂.

In one embodiment, compound of formula AVA-1 has one, preferably more, or more preferably all, of the following features:
i) WAR^{W2} is halo, C1-C6 alkyl, CF₃, CN, or phenyl optionally substituted with up to 3 substituents selected from C1-C4 alkyl, —O(C1-C4 alkyl), or halo;
ii) WAR^{W4} is CF₃, halo, C1-C6 alkyl, or C6-C10 cycloaliphatic; and
iii) WAR^{W5} is OH, NH₂, NH(C1-C6 alkyl), or N(C1-C6 alkyl).

In one embodiment, X-AR^{X} is at the 6-position of the quinolinyl ring. In certain embodiments, X-AR^{X} taken together is C1-C6 alkyl, —O—(C1-C6 alkyl), or halo.

In one embodiment, X-AR^{X} is at the 5-position of the quinolinyl ring. In certain embodiments, X-AR^{X} taken together is —OH.

In another embodiment, the present invention provides compounds of formula AVA-1, wherein WAR^{W4} and WAR^{W5} taken together form a 5-7 membered ring containing 0-3 three heteroatoms selected from N, O, or S, wherein said ring is optionally substituted with up to three WAR^{W} substituents.

In certain embodiments, WAR^{W4} and WAR^{W5} taken together form an optionally substituted 5-7 membered saturated, unsaturated, or aromatic ring containing 0 heteroatoms. In other embodiments, WAR^{W4} and WAR^{W5} taken together form an optionally substituted 5-7 membered ring containing 1-3 heteroatoms selected from N, O, or S. In certain other embodiments, WAR^{W4} and WAR^{W5} taken together form an optionally substituted saturated, unsaturated, or aromatic 5-7 membered ring containing 1 nitrogen heteroatom. In certain other embodiments, WAR^{W4} and WAR^{W5} taken together form an optionally substituted 5-7 membered ring containing 1 oxygen heteroatom.

In another embodiment, the present invention provides compounds of formula AVA-2:

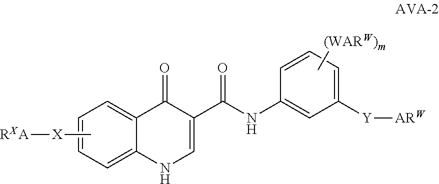

wherein:
Y is CH₂, C(O)O, C(O), or S(O)₂;
m is 0-4; and
X, AR^{X}, W, and AR^{W} are as defined above.

In one embodiment, compounds of formula AVA-2 have y occurrences of X-AR^{X}, wherein y is 0-4. In one embodiment, y is 0. Or, y is 1. Or, y is 2.

In one embodiment, Y is C(O). In another embodiment, Y is C(O)O. Or, Y is S(O)₂. Or, Y is CH₂.

In one embodiment, m is 1 or 2. Or, m is 1. Or, m is 0.

In one embodiment, W is a bond.

In another embodiment, AR^{W} is C1-C6 aliphatic, halo, CF₃, or phenyl optionally substituted with C1-C6 alkyl, halo, cyano, or CF₃, wherein up to two methylene units of said C1-C6 aliphatic or C1-C6 alkyl is optionally replaced with —CO—, —CONAR′—, —CO₂—, —OCO—, —NAR′CO₂—, —O—, —NAR′CONAR′—, —OCONAR′—, —NAR′CO—, —S—, —NAR′—, —SO₂NAR′—, NAR′SO₂—, or —NAR′SO₂NAR′—. In another embodiment, AR′ above is C1-C4 alkyl.

Exemplary embodiments of WAR^{W} include methyl, ethyl, propyl, tert-butyl, or 2-ethoxyphenyl.

In another embodiment, AR^{W} in Y-AR^{W} is C1-C6 aliphatic optionally substituted with N(AR″)₂, wherein AR″ is hydrogen, C1-C6 alkyl, or two R″ taken together form a 5-7 membered heterocyclic ring with up to 2 additional heteroatoms selected from O, S, or NAR′. Exemplary such heterocyclic rings include pyrrolidinyl, piperidyl, morpholinyl, or thiomorpholinyl.

In another embodiment, the present invention provides compounds of formula AVA-3:

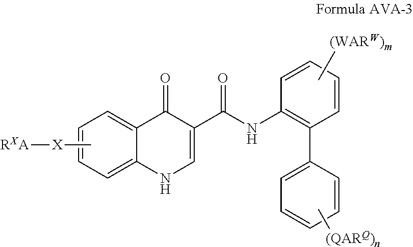

wherein:
Q is W;
AR^{Q} is AR^{W};
m is 0-4;
n is 0-4; and
X, AR^{X}, W, and AR^{W} are as defined above.

In one embodiment, compounds of formula AVA-3 have y occurrences of X-AR^{X}, wherein y is 0-4. In one embodiment, y is 0. Or, y is 1. Or, y is 2.

In one embodiment, n is 0-2.

In another embodiment, m is 0-2. In one embodiment, m is 0. In one embodiment, m is 1. Or, m is 2.

In one embodiment, QAR^{Q} taken together is halo, CF₃, OCF₃, CN, C1-C6 aliphatic, O—C1-C6 aliphatic, O-phenyl, NH(C1-C6 aliphatic), or N(C1-C6 aliphatic)₂, wherein said aliphatic and phenyl are optionally substituted with up to three substituents selected from C1-C6 alkyl, O—C1-C6 alkyl, halo, cyano, OH, or CF₃, wherein up to two methylene units of said C1-C6 aliphatic or C1-C6 alkyl is optionally replaced with —CO—, —CONAR′—, —CO₂—, —OCO—, —NAR′CO₂—, —O—, —NAR′CONAR′—, —OCONAR′—, —NAR′CO—, —S—, —NAR′—, SOAR′, SO₂AR′, —SO₂NAR′—, NAR′SO₂—, or —NAR′SO₂NAR′—. In another embodiment, AR′ above is C1-C4 alkyl.

Exemplary QAR^{Q} include methyl, isopropyl, sec-butyl, hydroxymethyl, CF₃, NMe₂, CN, CH₂CN, fluoro, chloro, OEt, OMe, SMe, OCF₃, OPh, C(O)OMe, C(O)O-iPr, S(O)Me, NHC(O)Me, or S(O)₂Me.

In another embodiment, the present invention provides compounds of formula AVA-4:

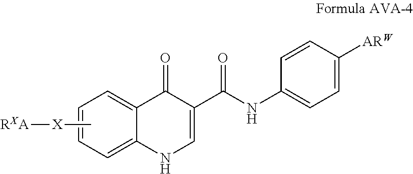

wherein X, AR^{X}, and AR^{W} are as defined above.

In one embodiment, compounds of formula AVA-4 have y occurrences of X-AR^{X}, wherein y is 0-4. In one embodiment, y is 0. Or, y is 1. Or, y is 2.

In one embodiment, AR^{W} is C1-C12 aliphatic, C5-C10 cycloaliphatic, or C5-C7 heterocyclic ring, wherein said aliphatic, cycloaliphatic, or heterocyclic ring is optionally substituted with up to three substituents selected from C1-C6 alkyl, halo, cyano, oxo, OH, or CF₃, wherein up to two methylene units of said C1-C6 aliphatic or C1-C6 alkyl is optionally replaced with —CO—, —CONAR′—, —CO₂—, —OCO—, —NAR′CO₂—, —O—, —NAR′CONAR′—, —OCONAR′—, —NAR′CO—, —S—, —NAR′—, —SO₂NAR′—, NAR′SO₂—, or —NAR′SO₂NAR′—. In another embodiment, AR′ above is C1-C4 alkyl.

Exemplary AR^{W} includes methyl, ethyl, n-propyl, isopropyl, n-butyl, sec-butyl, t-butyl, n-pentyl, vinyl, cyanomethyl, hydroxymethyl, hydroxyethyl, hydroxybutyl, cyclohexyl, adamantyl, or —C(CH₃)₂—NHC(O)O-T, wherein T is C1-C4 alkyl, methoxyethyl, or tetrahydrofuranylmethyl.

In another embodiment, the present invention provides compounds of formula AVA-5:

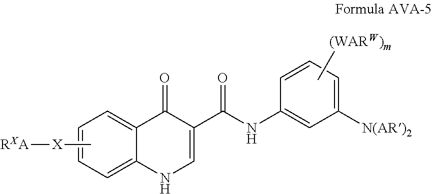

wherein:
m is 0-4; and
X, AR^{X}, W, AR^{W}, and R′ are as defined above.
In one embodiment, compounds of formula AVA-5 have y occurrences of X-AR^{X}, wherein y is 0-4. In one embodiment, y is 0. Or, y is 1. Or, y is 2.

In one embodiment, m is 0-2. Or, m is 1. Or, m is 2.

In another embodiment, both AR′ are hydrogen. Or, one AR′ is hydrogen and the other AR′ is C1-C4 alkyl, e.g., methyl. Or, both AR′ are C1-C4 alkyl, e.g., methyl.

In another embodiment, m is 1 or 2, and AR″ is halo, CF₃, CN, C1-C6 aliphatic, O—C1-C6 aliphatic, or phenyl, wherein said aliphatic and phenyl are optionally substituted with up to three substituents selected from C1-C6 alkyl, O—C1-C6 alkyl, halo, cyano, OH, or CF₃, wherein up to two methylene units of said C1-C6 aliphatic or C1-C6 alkyl is optionally replaced with —CO—, —CONAR′—, —CO₂—, —OCO—, —NAR′CO₂—, —O—, —NAR′CONAR′—, —OCONAR′—, —NAR′CO—, —S—, —NAR′—, —SO₂NAR′—, NAR′SO₂—, or —NAR′SO₂NAR′—. In another embodiment, AR′ above is C1-C4 alkyl.

Exemplary embodiments of AR″ include chloro, CF₃, OCF₃, methyl, ethyl, n-propyl, isopropyl, n-butyl, t-butyl, methoxy, ethoxy, propyloxy, or 2-ethoxyphenyl.

In another embodiment, the present invention provides compounds of Formula AVA-6:

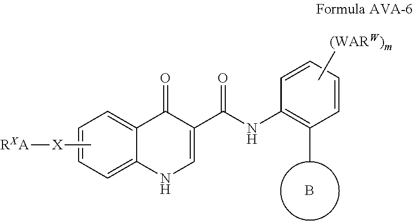

wherein:
ring B is a 5-7 membered monocyclic or bicyclic, heterocyclic or heteroaryl ring optionally substituted with up to n occurrences of -Q-AR^{Q}, wherein n is 0-4, and Q and AR^{Q} are as defined above; and

Q, AR^{Q}, X, AR^{X}, W, and AR^{W} are as defined above.

In one embodiment, compounds of formula AVA-6 have y occurrences of X-AR^{X}, wherein y is 0-4. In one embodiment, y is 0. Or, y is 1. Or, y is 2.

In one embodiment, m is 0-2. Or, m is 0. Or m is 1.

In one embodiment, n is 0-2. Or, n is 0. Or, n is 1.

In another embodiment, ring B is a 5-7 membered monocyclic, heterocyclic ring having up to 2 heteroatoms selected from O, S, or N, optionally substituted with up to n occurrences of -Q-AR^{Q}. Exemplary heterocyclic rings include N-morpholinyl, N-piperidinyl, 4-benzoyl-piperazin-1-yl, pyrrolidin-1-yl, or 4-methyl-piperidin-1-yl.

In another embodiment, ring B is a 5-6 membered monocyclic, heteroaryl ring having up to 2 heteroatoms selected from O, S, or N, optionally substituted with up to n occurrences of -Q-AR^{Q}. Exemplary such rings include benzimidazol-2-yl, 5-methyl-furan-2-yl, 2,5-dimethyl-pyrrol-1-yl, pyridine-4-yl, indol-5-yl, indol-2-yl, 2,4-dimethoxy-pyrimidin-5-yl, furan-2-yl, furan-3-yl, 2-acyl-thien-2-yl, benzothiophen-2-yl, 4-methyl-thien-2-yl, 5-cyano-thien-2-yl, 3-chloro-5-trifluoromethyl-pyridin-2-yl.

In another embodiment, the present invention provides compounds of formula AVB-1:

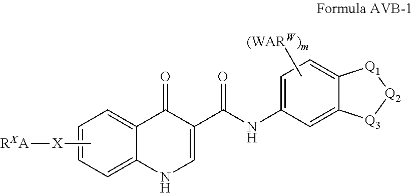

wherein:
one of Q₁ and Q₃ is N(WAR^{W}) and the other of Q₁ and Q₃ is selected from O, S, or N(WAR′);
Q₂ is C(O), CH₂—C(O), C(O)—CH₂, CH₂, CH₂—CH₂, CF₂, or CF₂—CF₂;
m is 0-3; and
X, W, AR^{X}, and AR^{W} are as defined above.

In one embodiment, compounds of formula AVB-1 have y occurrences of X-AR^{X}, wherein y is 0-4. In one embodiment, y is 0. Or, y is 1. Or, y is 2.

In one embodiment, Q₃ is N(WAR^{W}); exemplary WAR^{W} include hydrogen, C1-C6 aliphatic, C(O)C1-C6 aliphatic, or C(O)OC1-C6 aliphatic.

In another embodiment, Q₃ is N(WAR^{W}), Q₂ is C(O), CH₂, CH₂—CH₂, and Q₁ is 0.

In another embodiment, the present invention provides compounds of formula AVB-2:

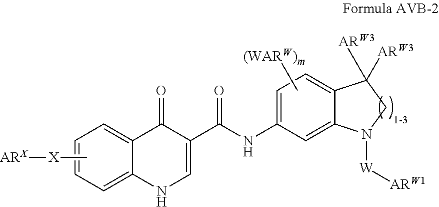

wherein:
AR^{W1} is hydrogen or C1-C6 aliphatic;
each of AR^{W3} is hydrogen or C1-C6 aliphatic; or
both AR^{W3} taken together form a C3-C6 cycloalkyl or heterocyclic ring having up to two heteroatoms selected from O, S, or NAR′, wherein said ring is optionally substituted with up to two WAR^{W} substituents;
m is 0-4; and
X, AR^{X}, W, and AR^{W} are as defined above.

In one embodiment, compounds of formula AVB-2 have y occurrences of X-AR^{X}, wherein y is 0-4. In one embodiment, y is 0. Or, y is 1. Or, y is 2.

In one embodiment, WAR^{W1} is hydrogen, C1-C6 aliphatic, C(O)C1-C6 aliphatic, or C(O)OC1-C6 aliphatic.

In another embodiment, each AR^{W3} is hydrogen, C1-C4 alkyl. Or, both AR^{W3} taken together form a C3-C6 cycloaliphatic ring or 5-7 membered heterocyclic ring having up to two heteroatoms selected from O, S, or N, wherein said cycloaliphatic or heterocyclic ring is optionally substituted with up to three substitutents selected from WAR^{W1}. Exemplary such rings include cyclopropyl, cyclopentyl, optionally substituted piperidyl, etc.

In another embodiment, the present invention provides compounds of formula AVB-3:

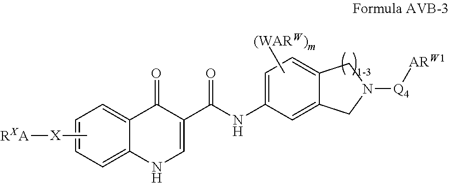

wherein:
Q₄ is a bond, C(O), C(O)O, or S(O)₂;
AR^{W1} is hydrogen or C1-C6 aliphatic;
m is 0-4; and
X, W, AR^{W}, and AR^{X} are as defined above.

In one embodiment, compounds of formula AVB-3 have y occurrences of X-AR^{X}, wherein y is 0-4. In one embodiment, y is 0.

In one embodiment, Q₄ is C(O). Or Q₄ is C(O)O. In another embodiment, AR^{W1} is C1-C6 alkyl. Exemplary AR^{W1} include methyl, ethyl, or t-butyl.

In another embodiment, the present invention provides compounds of formula AVB-4:

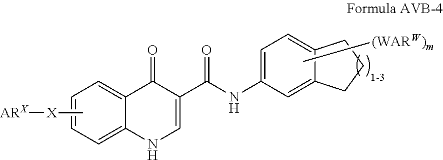

wherein:
m is 0-4; and
X, AR^{X}, W, and AR^{W} are as defined above.

In one embodiment, compounds of formula AVB-4 have y occurrences of X-AR^{X}, wherein y is 0-4. In one embodiment, y is 0. Or, y is 1. Or, y is 2.

In one embodiment, m is 0-2. Or, m is 0. Or, m is 1.

In another embodiment, said cycloaliphatic ring is a 5-membered ring. Or, said ring is a six-membered ring.

In another embodiment, the present invention provides compounds of formula AVB-5:

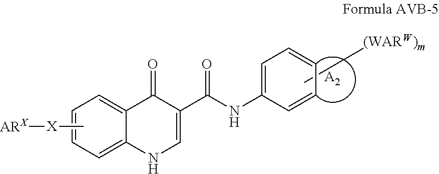

wherein:
ring A₂ is a phenyl or a 5-6 membered heteroaryl ring, wherein ring A₂ and the phenyl ring fused thereto together have up 4 substituents independently selected from WAR^{W};
m is 0-4; and
X, W, AR^{W} and AR^{X} are as defined above.

In one embodiment, compounds of formula AVB-5 have y occurrences of X-AR^{X}, wherein y is 0-4. In one embodiment, y is 0. Or, y is 1. Or, y is 2.

In one embodiment, ring A₂ is an optionally substituted 5-membered ring selected from pyrrolyl, furanyl, thienyl, pyrazolyl, imidazolyl, thiazolyl, oxazolyl, thiadiazolyl, oxadiazolyl, or triazolyl.

In one embodiment, ring A₂ is an optionally substituted 5-membered ring selected from pyrrolyl, pyrazolyl, thiadiazolyl, imidazolyl, oxazolyl, or triazolyl. Exemplary such rings include:

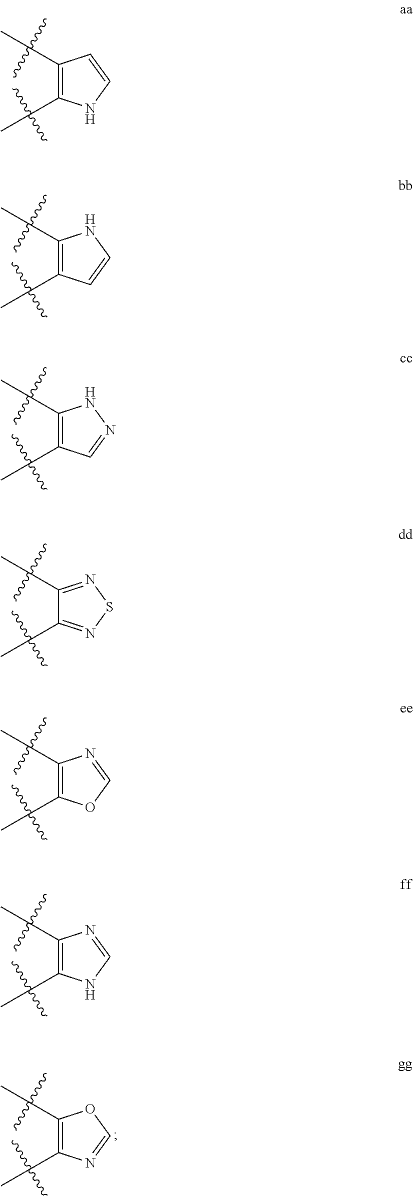

wherein said ring is optionally substituted as set forth above.

In another embodiment, ring A₂ is an optionally substituted 6-membered ring. Exemplary such rings include pyridyl, pyrazinyl, or triazinyl. In another embodiment, said ring is an optionally pyridyl.

In one embodiment, ring A₂ is phenyl.

In another embodiment, ring A₂ is pyrrolyl, pyrazolyl, pyridyl, or thiadiazolyl.

Exemplary W in formula V-B-5 includes a bond, C(O), C(O)O or C1-C6 alkylene.

Exemplary AR^{W} in formula V-B-5 include cyano, halo, C1-C6 aliphatic, C3-C6 cycloaliphatic, aryl, 5-7 membered heterocyclic ring having up to two heteroatoms selected from O, S, or N, wherein said aliphatic, phenyl, and heterocyclic are independently and optionally substituted with up to three substituents selected from C1-C6 alkyl, O—C1-C6 alkyl, halo, cyano, OH, or CF₃, wherein up to two methylene units of said C1-C6 aliphatic or C1-C6 alkyl is optionally replaced with —CO—, —CONAR′—, —CO₂—, —OCO—, —NAR′CO₂—, —O—, —NAR′CONAR′—, —OCONAR′—, —NAR′CO—, —S—, —NAR′—, —SO₂NAR′—, NAR′SO₂—, or —NAR′SO₂NAR′—. In another embodiment, AR′ above is C1-C4 alkyl.

In one embodiment, the present invention provides compounds of formula AVB-5-a:

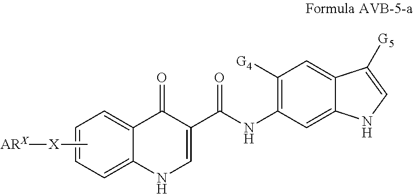

wherein:
G₄ is hydrogen, halo, CN, CF₃, CHF₂, CH₂F, optionally substituted C1-C6 aliphatic, aryl-C1-C6 alkyl, or a phenyl, wherein G₄ is optionally substituted with up to 4 WAR^{W} substituents; wherein up to two methylene units of said C1-C6 aliphatic or C1-C6 alkyl is optionally replaced with —CO—, —CONAR′—, —CO₂—, —OCO—, —NAR′CO₂—, —O—, —NAR′CONAR′—, —OCONAR′—, —NAR′CO—, —S—, —NAR′—, —SO₂NAR′—, NAR′SO₂—, or —NAR′SO₂NAR′—;
G₅ is hydrogen or an optionally substituted C1-C6 aliphatic;
wherein said indole ring system is further optionally substituted with up to 3 substituents independently selected from WAR^{W}.

In one embodiment, compounds of formula AVB-5-a have y occurrences of X-AR^{X}, wherein y is 0-4. In one embodiment, y is 0. Or, y is 1. Or, y is 2.

In one embodiment, G₄ is hydrogen. Or, G₅ is hydrogen.

In another embodiment, G₄ is hydrogen, and G₅ is C1-C6 aliphatic, wherein said aliphatic is optionally substituted with C1-C6 alkyl, halo, cyano, or CF₃, and wherein up to two methylene units of said C1-C6 aliphatic or C1-C6 alkyl is optionally replaced with —CO—, —CONAR′—, —CO₂—, —OCO—, —NAR′CO₂—, —O—, —NAR′CONAR′—, —OCONAR′—, —NAR′CO—, —S—, —NAR′—, —SO₂NAR′—, NAR′SO₂—, or —NAR′SO₂NAR′—. In another embodiment, AR′ above is C1-C4 alkyl.

In another embodiment, G₄ is hydrogen, and G₅ is cyano, methyl, ethyl, propyl, isopropyl, butyl, sec-butyl, t-butyl, cyanomethyl, methoxyethyl, CH₂C(O)OMe, (CH₂)₂—NHC(O)O-tert-butyl, or cyclopentyl.

In another embodiment, G₅ is hydrogen, and G₄ is halo, C1-C6 aliphatic or phenyl, wherein said aliphatic or phenyl is optionally substituted with C1-C6 alkyl, halo, cyano, or CF₃, wherein up to two methylene units of said C1-C6 aliphatic or C1-C6 alkyl is optionally replaced with —CO—, —CONAR′—, —CO₂—, —OCO—, —NAR′CO₂—, —O—, —NAR′CONAR′—, —OCONAR′—, —NAR′CO—, —S—, —NAR′—, —SO₂NAR′—, NAR′SO₂—, or —NAR′SO₂NAR′—. In another embodiment, AR′ above is C1-C4 alkyl.

In another embodiment, G₅ is hydrogen, and G₄ is halo, CF₃, ethoxycarbonyl, t-butyl, 2-methoxyphenyl, 2-ethoxyphenyl, (4-C(O)NH(CH₂)₂—NMe₂)-phenyl, 2-methoxy-4-chloro-phenyl, pyridine-3-yl, 4-isopropylphenyl, 2,6-dimethoxyphenyl, sec-butylaminocarbonyl, ethyl, t-butyl, or piperidin-1-ylcarbonyl.

In another embodiment, G₄ and G₅ are both hydrogen, and the nitrogen ring atom of said indole ring is substituted with C1-C6 aliphatic, C(O)(C1-C6 aliphatic), or benzyl, wherein said aliphatic or benzyl is optionally substituted with C1-C6 alkyl, halo, cyano, or CF₃, wherein up to two methylene units of said C1-C6 aliphatic or C1-C6 alkyl is optionally replaced with —CO—, —CONAR′—, —CO₂—, —OCO—, —NAR′COᵣ, —O—, —NAR′CONAR′—, —OCONAR′—, —NAR′CO—, —S—, —NAR′—, —SO₂NAR′—, NAR′SO₂—, or —NAR′SO₂NAR′—. In another embodiment, AR′ above is C1-C4 alkyl.

In another embodiment, G₄ and G₅ are both hydrogen, and the nitrogen ring atom of said indole ring is substituted with acyl, benzyl, C(O)CH₂N(Me)C(O)CH₂NHMe, or ethoxycarbonyl.

In another embodiment, the present invention provides compounds of formula AI′:

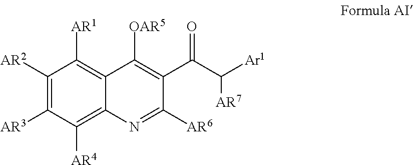

or pharmaceutically acceptable salts thereof,
wherein AR¹, AR², AR³, AR⁴, AR⁵, AR⁶, AR⁷, and Ar¹ is as defined above for compounds of formula AI′.

In one embodiment, each of AR¹, AR², AR³, AR⁴, AR⁵, AR⁶, AR⁷, and Ar¹ in compounds of formula AI′ is independently as defined above for any of the embodiments of compounds of Formula A.

Representative compounds of the present invention are set forth below in Table II.A-1 below.

TABLE II.A-1
of Column A Compounds Useful In The Present Combination Compositions

| Cmpd | |
|---|---|
| No. | Name |
| 1 | N-[5-(5-chloro-2-methoxy-phenyl)-1H-indol-6-yl]-4-oxo-1H-quinoline-3-carboxamide |
| 2 | N-(3-methoxy-4-tert-butyl-phenyl)-4-oxo-1H-quinoline-3-carboxamide |
| 3 | N-[2-(2-methoxyphenoxy)-5-(trifluoromethyl)phenyl]-4-oxo-1H-quinoline-3-carboxamide |
| 4 | N-(2-morpholinophenyl)-4-oxo-1H-quinoline-3-carboxamide |
| 5 | N-[4-(2-hydroxy-1,1-dimethyl-ethyl)phenyl]-4-oxo-1H-quinoline-3-carboxamide |
| 6 | N-[3-(hydroxymethyl)-4-tert-butyl-phenyl]-4-oxo-1H-quinoline-3-carboxamide |
| 7 | N-(4-benzoylamino-2,5-diethoxy-phenyl)-4-oxo-1H-quinoline-3-carboxamide |
| 8 | N-(3-amino-4-ethyl-phenyl)-4-oxo-1H-quinoline-3-carboxamide |
| 9 | 4-oxo-N-(3-sulfamoylphenyl)-1H-quinoline-3-carboxamide |
| 10 | 1,4-dihydro-N-(2,3,4,5-tetrahydro-1H-benzo[b]azepin-8-yl)-4-oxoquinoline-3-carboxamide |
| 11 | 4-oxo-N-[2-[2-(trifluoromethyl)phenyl]phenyl]-1H-quinoline-3-carboxamide |
| 12 | N-[2-(4-dimethylaminophenyl)phenyl]-4-oxo-1H-quinoline-3-carboxamide |
| 13 | N-(3-cyano-4-tert-butyl-phenyl)-4-oxo-1H-quinoline-3-carboxamide |
| 14 | [5-[(4-oxo-1H-quinolin-3-yl)carbonylamino]-2-tert-butyl-phenyl]aminoformic acid methyl ester |
| 15 | N-(2-methoxy-3-pyridyl)-4-oxo-1H-quinoline-3-carboxamide |
| 16 | 4-oxo-N-(2-propylphenyl)-1H-quinoline-3-carboxamide |
| 17 | N-(5-amino-2-propoxy-phenyl)-4-oxo-1H-quinoline-3-carboxamide |
| 18 | N-(9H-fluoren-1-yl)-4-oxo-1H-quinoline-3-carboxamide |
| 19 | 4-oxo-N-(2-quinolyl)-1H-quinoline-3-carboxamide |
| 20 | N-[2-(2-methylphenoxy)phenyl]-4-oxo-1H-quinoline-3-carboxamide |
| 21 | 4-oxo-N-[4-(2-pyridylsulfamoyl)phenyl]-1H-quinoline-3-carboxamide |
| 22 | 4-Oxo-1,4-dihydro-quinoline-3-carboxylic acid N-(1′,2′-dihydrospiro[cyclopropane-1,3′- |
| | [3H]indol]-6′-yl)-amide |
| 23 | N-[2-(2-ethoxyphenyl)-5-hydroxy-4-tert-butyl-phenyl]-4-oxo-1H-quinoline-3-carboxamide |
| 24 | 4-oxo-N-(3-pyrrolidin-1-ylsulfonylphenyl)-1H-quinoline-3-carboxamide |
| 25 | N-[2-(3-acetylaminophenyl)phenyl]-4-oxo-1H-quinoline-3-carboxamide |
| 26 | 4-oxo-N-[2-(1-piperidyl)phenyl]-1H-quinoline-3-carboxamide |
| 27 | N-[1-[2-[methyl-(2-methylaminoacetyl)-amino]acetyl]-1H-indol-6-yl]-4-oxo-1H-quinoline-3- |
| | carboxamide |
| 28 | [2-methyl-2-[4-[(4-oxo-1H-quinolin-3-yl)carbonylamino]phenyl]-propyl]aminoformic acid 2- |
| | methoxyethyl ester |
| 29 | 1-isopropyl-4-oxo-N-phenyl-1H-quinoline-3-carboxamide |
| 30 | [2-isopropyl-5-[(4-oxo-1H-quinolin-3-yl)carbonylamino]phenyl]aminoformic acid methyl ester |
| 31 | 4-oxo-N-(p-tolyl)-1H-quinoline-3-carboxamide |
| 32 | N-(5-chloro-1H-indol-6-yl)-4-oxo-1H-quinoline-3-carboxamide |
| 33 | N-(1H-indol-6-yl)-4-oxo-1H-quinoline-3-carboxamide |
| 34 | N-[4-(1,1-diethylpropyl)-2-fluoro-5-hydroxy-phenyl]-4-hydroxy-quinoline-3-carboxamide |
| 35 | 1,4-dihydro-N-(2,3,4,5-tetrahydro-5,5-dimethyl-1H-benzo[b]azepin-8-yl)-4-oxoquinoline-3- |
| | carboxamide |
| 36 | N-(2-isopropylphenyl)-4-oxo-1H-quinoline-3-carboxamide |
| 37 | N-(1H-indol-7-yl)-4-oxo-1H-quinoline-3-carboxamide |
| 38 | N-[2-(1H-indol-2-yl)phenyl]-4-oxo-1H-quinoline-3-carboxamide |
| 39 | [3-[(2,4-dimethoxy-3-quinolyl)carbonylamino]-4-tert-butyl-phenyl]aminoformic acid tert-butyl |
| | ester |
| 40 | N-[2-(2-hydroxyethyl)phenyl]-4-oxo-1H-quinoline-3-carboxamide |
| 41 | N-(5-amino-2-propyl-phenyl)-4-oxo-1H-quinoline-3-carboxamide |
| 42 | N-[2-[[3-chloro-5-(trifluoromethyl)-2-pyridyl]oxy]phenyl]-4-oxo-1H-quinoline-3-carboxamide |
| 43 | N-[2-(3-ethoxyphenyl)-5-hydroxy-4-tert-butyl-phenyl]-4-oxo-1H-quinoline-3-carboxamide |
| 44 | N-(2-methylbenzothiazol-5-yl)-4-oxo-1H-quinoline-3-carboxamide |
| 45 | N-(2-cyano-3-fluoro-phenyl)-4-oxo-1H-quinoline-3-carboxamide |
| 46 | N-[3-chloro-5-(2-morpholinoethylsulfonylamino)phenyl]-4-oxo-1H-quinoline-3-carboxamide |
| 47 | N-[4-isopropyl-2-(trifluoromethyl)phenyl]-4-oxo-1H-quinoline-3-carboxamide |
| 48 | N-(5-chloro-2-fluoro-phenyl)-4-oxo-1H-quinoline-3-carboxamide |
| 49 | N-[2-(2,6-dimethoxyphenyl)phenyl]-4-oxo-1H-quinoline-3-carboxamide |
| 50 | 4-oxo-N-(2,4,6-trimethylphenyl)-1H-quinoline-3-carboxamide |
| 51 | 6-[(4-methyl-1-piperidyl)sulfonyl]-4-oxo-N-(5-tert-butyl-1H-indol-6-yl)-1H-quinoline-3- |
| | carboxamide |
| 52 | N-[2-(m-tolyl)phenyl]-4-oxo-1H-quinoline-3-carboxamide |
| 53 | 4-oxo-N-(4-pyridyl)-1H-quinoline-3-carboxamide |
| 54 | 4-oxo-N-(8-thia-7,9-diazabicyclo[4.3.0]nona-2,4,6,9-tetraen-5-yl)-1H-quinoline-3-carboxamide |
| 55 | N-(3-amino-2-methoxy-5-tert-butyl-phenyl)-4-oxo-1H-quinoline-3-carboxamide |
| 56 | 1,4-dihydro-N-(1,2,3,4-tetrahydro-6-hydroxynaphthalen-7-yl)-4-oxoquinoline-3-carboxamide |
| 57 | N-[4-(3-ethyl-2,6-dioxo-3-piperidyl)phenyl]-4-oxo-1H-quinoline-3-carboxamide |
| 58 | N-[3-amino-4-(trifluoromethoxy)phenyl]-4-oxo-1H-quinoline-3-carboxamide |
| 59 | N-[2-(5-isopropyl-2-methoxy-phenyl)phenyl]-4-oxo-1H-quinoline-3-carboxamide |
| 60 | [4-isopropyl-3-[(4-oxo-1H-quinolin-3-yl)carbonylamino]phenyl]aminoformic acid tert-butyl |
| | ester |
| 61 | N-(2,3-dimethylphenyl)-4-oxo-1H-quinoline-3-carboxamide |
| 62 | 4-oxo-N-[3-(trifluoromethoxy)phenyl]-1H-quinoline-3-carboxamide |
| 63 | N-[2-(2,4-difluorophenyl)phenyl]-4-oxo-1H-quinoline-3-carboxamide |
| 64 | 4-oxo-N-(2-oxo-1,3-dihydrobenzoimidazol-5-yl)-1H-quinoline-3-carboxamide |
| 65 | 4-oxo-N-[5-(3-pyridyl)-1H-indol-6-yl]-1H-quinoline-3-carboxamide |
| 66 | N-(2,2-difluorobenzo[1,3]dioxol-5-yl)-4-oxo-1H-quinoline-3-carboxamide |
| 67 | 6-ethyl-4-hydroxy-N-(1H-indol-6-yl)quinoline-3-carboxamide |
| 68 | 3-[2-[(4-oxo-1H-quinolin-3-yl)carbonylamino]phenyl]benzoic acid methyl ester |
| 69 | N-(3-amino-4-isopropyl-phenyl)-4-oxo-1H-quinoline-3-carboxamide |
| 70 | 4-oxo-N-[2-(4-pyridyl)phenyl]-1H-quinoline-3-carboxamide |
| 71 | 3-[2-[(4-oxo-1H-quinolin-3-yl)carbonylamino]phenyl]benzoic acid isopropyl ester |
| 72 | N-(2-ethylphenyl)-4-oxo-1H-quinoline-3-carboxamide |
| 73 | 4-oxo-N-(2-phenyl-3H-benzoimidazol-5-yl)-1H-quinoline-3-carboxamide |
| 74 | 4-oxo-N-[5-(trifluoromethyl)-2-pyridyl]-1H-quinoline-3-carboxamide |
| 75 | 4-oxo-N-(3-quinolyl)-1H-quinoline-3-carboxamide |
| 76 | N-[2-(3,4-difluorophenyl)phenyl]-4-oxo-1H-quinoline-3-carboxamide |
| 77 | N-(5-fluoro-1H-indol-6-yl)-4-oxo-1H-quinoline-3-carboxamide |
| 78 | 4-oxo-N-(2-sulfamoylphenyl)-1H-quinoline-3-carboxamide |
| 79 | N-[2-(4-fluoro-3-methyl-phenyl)phenyl]-4-oxo-1H-quinoline-3-carboxamide |
| 80 | N-(2-methoxyphenyl)-4-oxo-1H-quinoline-3-carboxamide |
| 81 | 4-oxo-N-(3-propionylaminophenyl)-1H-quinoline-3-carboxamide |
| 82 | N-(4-diethylamino-2-methyl-phenyl)-4-oxo-1H-quinoline-3-carboxamide |
| 83 | N-[2-(3-cyanophenyl)phenyl]-4-oxo-1H-quinoline-3-carboxamide |
| 84 | N-(4-methyl-2-pyridyl)-4-oxo-1H-quinoline-3-carboxamide |
| 85 | N-[2-(3,4-dichlorophenyl)phenyl]-4-oxo-1H-quinoline-3-carboxamide |
| 86 | N-[4-[2-(aminomethyl)phenyl]phenyl]-4-oxo-1H-quinoline-3-carboxamide |
| 87 | 4-oxo-N-(3-phenoxyphenyl)-1H-quinoline-3-carboxamide |
| 88 | [2-methyl-2-[4-[(4-oxo-1H-quinolin-3-yl)carbonylamino]phenyl]-propyl]aminoformic acid tert- |
| | butyl ester |
| 89 | N-(2-cyano-5-methyl-phenyl)-4-oxo-1H-quinoline-3-carboxamide |
| 90 | 4-oxo-N-(2-tert-butylphenyl)-1H-quinoline-3-carboxamide |
| 91 | N-(3-chloro-2,6-diethyl-phenyl)-4-oxo-1H-quinoline-3-carboxamide |
| 92 | N-[2-fluoro-5-hydroxy-4-(1-methylcyclohexyl)-phenyl]-4-oxo-1H-quinoline-3-carboxamide |
| 93 | N-[2-(5-cyano-2-thienyl)phenyl]-4-oxo-1H-quinoline-3-carboxamide |
| 94 | N-(5-amino-2-methyl-phenyl)-4-oxo-1H-quinoline-3-carboxamide |
| 95 | N-(2-cyanophenyl)-4-oxo-1H-quinoline-3-carboxamide |
| 96 | N-[3-(cyanomethyl)-1H-indol-6-yl]-4-oxo-1H-quinoline-3-carboxamide |
| 97 | N-[2-(2,4-dimethoxypyrimidin-5-yl)phenyl]-4-oxo-1H-quinoline-3-carboxamide |
| 98 | N-(5-dimethylamino-2-propyl-phenyl)-4-oxo-1H-quinoline-3-carboxamide |
| 99 | 4-oxo-N-(4-pentylphenyl)-1H-quinoline-3-carboxamide |
| 100 | N-(1H-indol-4-yl)-4-oxo-1H-quinoline-3-carboxamide |
| 101 | N-(5-amino-2-isopropyl-phenyl)-4-oxo-1H-quinoline-3-carboxamide |
| 102 | N-[2-[3-(4-chlorophenyl)-1,2,4-oxadiazol-5-yl]phenyl]-4-oxo-1H-quinoline-3-carboxamide |
| 103 | 6-fluoro-N-(5-hydroxy-2,4-ditert-butyl-phenyl)-4-oxo-1H-quinoline-3-carboxamide |
| 104 | N-(2-methyl-1H-indol-6-yl)-4-oxo-1H-quinoline-3-carboxamide |
| 105 | 1,4-dihydro-N-(3,4-dihydro-2H-benzo[b][1,4]oxazin-6-yl)-4-oxoquinoline-3-carboxamide |
| 106 | N-(2-cyano-4,5-dimethoxy-phenyl)-4-oxo-1H-quinoline-3-carboxamide |
| 107 | 7-[(4-oxo-1H-quinolin-3-yl)carbonylamino]-1,2,3,4-tetrahydroisoquinoline-2-carboxylic acid |
| | tert-butyl ester |
| 108 | 4,4-dimethyl-7-[(4-oxo-1H-quinolin-3-yl)carbonylamino]-1,2,3,4-tetrahydroquinoline-1- |
| | carboxylic acid tert-butyl ester |
| 109 | N-(1-acetyl-2,3,4,5-tetrahydro-5,5-dimethyl-1H-benzo[b]azepin-8-yl)-1,4-dihydro-4- |
| | oxoquinoline-3-carboxamide |
| 110 | N-[4-(cyanomethyl)phenyl]-4-oxo-1H-quinoline-3-carboxamide |
| 111 | 4-oxo-N-[2-(trifluoromethyl)phenyl]-1H-quinoline-3-carboxamide |
| 112 | 6-ethoxy-4-hydroxy-N-(1H-indol-6-yl)quinoline-3-carboxamide |
| 113 | N-(3-methyl-1H-indol-6-yl)-4-oxo-1H-quinoline-3-carboxamide |
| 114 | [4-(2-ethoxyphenyl)-3-[(4-oxo-1H-quinolin-3-yl)carbonylamino]phenyl]aminoformic acid tert- |
| | butyl ester |
| 115 | N-[2-(2-furyl)phenyl]-4-oxo-1H-quinoline-3-carboxamide |
| 116 | 5-hydroxy-N-(1H-indol-6-yl)-4-oxo-1H-quinoline-3-carboxamide |
| 117 | N-(3-dimethylamino-4-isopropyl-phenyl)-4-oxo-1H-quinoline-3-carboxamide |
| 118 | N-[2-(1H-indol-5-yl)phenyl]-4-oxo-1H-quinoline-3-carboxamide |
| 119 | [2-methyl-2-[4-[(4-oxo-1H-quinolin-3-yl)carbonylamino]phenyl]-propyl]aminoformic acid |
| | ethyl ester |
| 120 | N-(2-methoxy-5-methyl-phenyl)-4-oxo-1H-quinoline-3-carboxamide |
| 121 | N-(3,4-dichlorophenyl)-4-oxo-1H-quinoline-3-carboxamide |
| 122 | N-(3,4-dimethoxyphenyl)-4-oxo-1H-quinoline-3-carboxamide |
| 123 | N-[2-(3-furyl)phenyl]-4-oxo-1H-quinoline-3-carboxamide |
| 124 | 6-fluoro-4-oxo-N-(5-tert-butyl-1H-indol-6-yl)-1H-quinoline-3-carboxamide |
| 125 | N-(6-ethyl-2-pyridyl)-4-oxo-1H-quinoline-3-carboxamide |
| 126 | N-[3-hydroxy-4-[2-(2-methoxyethoxy)-1,1-dimethyl-ethyl]-phenyl]-4-oxo-1H-quinoline-3- |
| | carboxamide |
| 127 | [5-[(4-oxo-1H-quinolin-3-yl)carbonylamino]-2-tert-butyl-phenyl]aminoformic acid ethyl ester |
| 128 | 1,6-dimethyl-4-oxo-N-(2-phenylphenyl)-1H-quinoline-3-carboxamide |
| 129 | [2-ethyl-5-[(4-oxo-1H-quinolin-3-yl)carbonylamino]phenyl]aminoformic acid methyl ester |
| 130 | 4-hydroxy-N-(1H-indol-6-yl)-5,7-bis(trifluoromethyl)quinoline-3-carboxamide |
| 131 | N-(3-amino-5-chloro-phenyl)-4-oxo-1H-quinoline-3-carboxamide |
| 132 | N-(5-acetylamino-2-ethoxy-phenyl)-4-oxo-1H-quinoline-3-carboxamide |
| 133 | N-[3-chloro-5-[2-(1-piperidyl)ethylsulfonylamino]phenyl]-4-oxo-1H-quinoline-3-carboxamide |
| 134 | N-[2-(4-methylsulfinylphenyl)phenyl]-4-oxo-1H-quinoline-3-carboxamide |
| 135 | N-(2-benzo[1,3]dioxol-5-ylphenyl)-4-oxo-1H-quinoline-3-carboxamide |
| 136 | N-(2-hydroxy-3,5-ditert-butyl-phenyl)-4-oxo-1H-quinoline-3-carboxamide |
| 137 | 6-[(4-fluorophenyl)-methyl-sulfamoyl]-N-(5-hydroxy-2,4-ditert-butyl-phenyl)-4-oxo-1H- |
| | quinoline-3-carboxamide |
| 138 | N-[2-(3,5-difluorophenyl)phenyl]-4-oxo-1H-quinoline-3-carboxamide |
| 139 | N-[2-(2,4-dichlorophenyl)phenyl]-4-oxo-1H-quinoline-3-carboxamide |
| 140 | N-(4-cyclohexylphenyl)-4-oxo-1H-quinoline-3-carboxamide |
| 141 | [2-methyl-5-[(4-oxo-1H-quinolin-3-yl)carbonylamino]phenyl]aminoformic acid ethyl ester |
| 142 | 4-oxo-N-(2-sec-butylphenyl)-1H-quinoline-3-carboxamide |
| 143 | N-(2-fluoro-5-hydroxy-4-tert-butyl-phenyl)-4-oxo-1H-quinoline-3-carboxamide |
| 144 | N-(3-hydroxyphenyl)-4-oxo-1H-quinoline-3-carboxamide |
| 145 | 6-[(4-oxo-1H-quinolin-3-yl)carbonylamino]-1H-indole-4-carboxylic acid ethyl ester |
| 146 | 4-oxo-N-(1,7,9-triazabicyclo[4.3.0]nona-2,4,6,8-tetraen-5-yl)-1H-quinoline-3-carboxamide |
| 147 | N-[2-(4-fluorophenoxy)-3-pyridyl]-4-oxo-1H-quinoline-3-carboxamide |
| 148 | 4-oxo-N-[5-(1-piperidylcarbonyl)-1H-indol-6-yl]-1H-quinoline-3-carboxamide |
| 149 | N-(3-acetylamino-4-ethyl-phenyl)-4-oxo-1H-quinoline-3-carboxamide |
| 150 | 4-oxo-N-[4-[2,2,2-trifluoro-1-hydroxy-1-(trifluoromethyl)ethyl]phenyl]-1H-quinoline-3- |
| | carboxamide |
| 151 | N-[2-(4-methyl-2-thienyl)phenyl]-4-oxo-1H-quinoline-3-carboxamide |
| 152 | 4-oxo-N-(2-oxo-3H-benzooxazol-6-yl)-1H-quinoline-3-carboxamide |
| 153 | N-[4-(1,1-diethyl-2,2-dimethyl-propyl)-2-fluoro-5-hydroxy-phenyl]-4-hydroxy-quinoline-3- |
| | carboxamide |
| 154 | N-[3,5-bis(trifluoromethyl)phenyl]-4-oxo-1H-quinoline-3-carboxamide |
| 155 | 4-oxo-N-(2-pyridyl)-1H-quinoline-3-carboxamide |
| 156 | 4-oxo-N-[2-[2-(trifluoromethoxy)phenyl]phenyl]-1H-quinoline-3-carboxamide |
| 157 | N-(2-ethyl-5-methylamino-phenyl)-4-oxo-1H-quinoline-3-carboxamide |
| 158 | 4-oxo-N-(5-phenyl-1H-indol-6-yl)-1H-quinoline-3-carboxamide |
| 159 | [7-[(4-oxo-1H-quinolin-3-yl)carbonylamino]tetralin-1-yl]aminoformic acid methyl ester |
| 160 | N-(3-amino-4-propyl-phenyl)-4-oxo-1H-quinoline-3-carboxamide |
| 161 | N-[3-(2-ethoxyethoxy)-4-tert-butyl-phenyl]-4-oxo-1H-quinoline-3-carboxamide |
| 162 | N-(6-methoxy-3-pyridyl)-4-oxo-1H-quinoline-3-carboxamide |
| 163 | N-[5-(aminomethyl)-2-(2-ethoxyphenyl)-phenyl]-4-oxo-1H-quinoline-3-carboxamide |
| 164 | 4-oxo-N-[3-(trifluoromethyl)phenyl]-1H-quinoline-3-carboxamide |
| 165 | 4-oxo-N-(4-sulfamoylphenyl)-1H-quinoline-3-carboxamide |
| 166 | 4-[2-[(4-oxo-1H-quinolin-3-yl)carbonylamino]phenyl]benzoic acid methyl ester |
| 167 | N-(3-amino-4-methyl-phenyl)-4-oxo-1H-quinoline-3-carboxamide |
| 168 | 4-oxo-N-(3-pyridyl)-1H-quinoline-3-carboxamide |
| 169 | N-(1-methyl-1H-indol-6-yl)-4-oxo-1H-quinoline-3-carboxamide |
| 170 | N-(5-chloro-2-pyridyl)-4-oxo-1H-quinoline-3-carboxamide |
| 171 | N-[2-(2,3-dichlorophenyl)phenyl]-4-oxo-1H-quinoline-3-carboxamide |
| 172 | N-(2-(benzo[b]thiophen-2-yl)phenyl)-1,4-dihydro-4-oxoquinoline-3-carboxamide |
| 173 | N-(6-methyl-2-pyridyl)-4-oxo-1H-quinoline-3-carboxamide |
| 174 | N-[2-(5-acetyl-2-thienyl)phenyl]-4-oxo-1H-quinoline-3-carboxamide |
| 175 | 4-Oxo-1,4-dihydro-quinoline-3-carboxylic acid N-(1′-Acetyl-1′,2′-dihydrospiro[cyclopropane- |
| | 1,3′-3H-indol]-6′-yl)-amide |
| 176 | 4-oxo-N-[4-(trifluoromethoxy)phenyl]-1H-quinoline-3-carboxamide |
| 177 | N-(2-butoxyphenyl)-4-oxo-1H-quinoline-3-carboxamide |
| 178 | 4-oxo-N-[2-(2-tert-butylphenoxy)phenyl]-1H-quinoline-3-carboxamide |
| 179 | N-(3-carbamoylphenyl)-4-oxo-1H-quinoline-3-carboxamide |
| 180 | N-(2-ethyl-6-methyl-phenyl)-4-oxo-1H-quinoline-3-carboxamide |
| 181 | 4-oxo-N-[2-(p-tolyl)phenyl]-1H-quinoline-3-carboxamide |
| 182 | N-[2-(4-fluorophenyl)phenyl]-4-oxo-1H-quinoline-3-carboxamide |
| 183 | 7-[(4-oxo-1H-quinolin-3-yl)carbonylamino]-1,2,3,4-tetrahydroquinoline-1-carboxylic acid tert- |
| | butyl ester |
| 184 | N-(1H-indol-6-yl)-4-oxo-2-(trifluoromethyl)-1H-quinoline-3-carboxamide |
| 185 | N-(3-morpholinosulfonylphenyl)-4-oxo-1H-quinoline-3-carboxamide |
| 186 | N-(3-cyclopentyl-1H-indol-6-yl)-4-oxo-1H-quinoline-3-carboxamide |
| 187 | N-(1-acetyl-1H-indol-6-yl)-4-oxo-1H-quinoline-3-carboxamide |
| 188 | 6-[(4-oxo-1H-quinolin-3-yl)carbonylamino]-1H-indole-5-carboxylic acid ethyl ester |
| 189 | N-(4-benzyloxyphenyl)-4-oxo-1H-quinoline-3-carboxamide |
| 190 | N-[2-(3-chloro-4-fluoro-phenyl)phenyl]-4-oxo-1H-quinoline-3-carboxamide |
| 191 | 4-oxo-N-(5-quinolyl)-1H-quinoline-3-carboxamide |
| 192 | N-(3-methyl-2-pyridyl)-4-oxo-1H-quinoline-3-carboxamide |
| 193 | N-(2,6-dimethoxy-3-pyridyl)-4-oxo-1H-quinoline-3-carboxamide |
| 194 | N-(4-cyanophenyl)-4-oxo-1H-quinoline-3-carboxamide |
| 195 | N-(5-methyl-2-pyridyl)-4-oxo-1H-quinoline-3-carboxamide |
| 196 | N-[5-(3,3-dimethylbutanoylamino)-2-tert-butyl-phenyl]-4-oxo-1H-quinoline-3-carboxamide |
| 197 | 4-oxo-N-[6-(trifluoromethyl)-3-pyridyl]-1H-quinoline-3-carboxamide |
| 198 | N-(4-fluorophenyl)-4-oxo-1H-quinoline-3-carboxamide |
| 199 | N-[2-(o-tolyl)phenyl]-4-oxo-1H-quinoline-3-carboxamide |
| 200 | 1,4-dihydro-N-(1,2,3,4-tetrahydro-1-hydroxynaphthalen-7-yl)-4-oxoquinoline-3-carboxamide |
| 201 | N-(2-cyano-3-methyl-phenyl)-4-oxo-1H-quinoline-3-carboxamide |
| 202 | N-[2-(5-chloro-2-methoxy-phenyl)phenyl]-4-oxo-1H-quinoline-3-carboxamide |
| 203 | N-(1-benzyl-1H-indol-6-yl)-4-oxo-1H-quinoline-3-carboxamide |
| 204 | N-(4,4-dimethylchroman-7-yl)-4-oxo-1H-quinoline-3-carboxamide |
| 205 | N-[2-(4-methoxyphenoxy)-5-(trifluoromethyl)phenyl]-4-oxo-1H-quinoline-3-carboxamide |
| 206 | N-[2-(2,3-dimethylphenoxy)-3-pyridyl]-4-oxo-1H-quinoline-3-carboxamide |
| 207 | 2-[6-[(4-oxo-1H-quinolin-3-yl)carbonylamino]-1H-indol-3-yl]acetic acid ethyl ester |
| 208 | N-[4-(2-adamantyl)-5-hydroxy-2-methyl-phenyl]-4-oxo-1H-quinoline-3-carboxamide |
| 209 | N-[4-(hydroxymethyl)phenyl]-4-oxo-1H-quinoline-3-carboxamide |
| 210 | 2,4-dimethoxy-N-(2-phenylphenyl)-quinoline-3-carboxamide |
| 211 | N-(2-methoxy-5-tert-butyl-phenyl)-4-oxo-1H-quinoline-3-carboxamide |
| 212 | N-[3-(3-methyl-5-oxo-1,4-dihydropyrazol-1-yl)phenyl]-4-oxo-1H-quinoline-3-carboxamide |
| 213 | N-[2-(2,5-dichlorophenyl)phenyl]-4-oxo-1H-quinoline-3-carboxamide |
| 214 | N-(3-methylsulfonylaminophenyl)-4-oxo-1H-quinoline-3-carboxamide |
| 215 | 4-oxo-N-phenyl-1H-quinoline-3-carboxamide |
| 216 | N-(3H-benzoimidazol-2-yl)-4-oxo-1H-quinoline-3-carboxamide |
| 217 | N-(1H-indazol-5-yl)-4-oxo-1H-quinoline-3-carboxamide |
| 218 | 6-fluoro-N-[2-fluoro-5-hydroxy-4-(1-methylcyclohexyl)-phenyl]-4-oxo-1H-quinoline-3- |
| | carboxamide |
| 219 | 4-oxo-N-pyrazin-2-yl-1H-quinoline-3-carboxamide |
| 220 | N-(2,3-dihydroxy-4,6-ditert-butyl-phenyl)-4-oxo-1H-quinoline-3-carboxamide |
| 221 | [5-[(4-oxo-1H-quinolin-3-yl)carbonylamino]-2-propyl-phenyl]aminoformic acid methyl ester |
| 222 | N-(3-chloro-2-cyano-phenyl)-4-oxo-1H-quinoline-3-carboxamide |
| 223 | N-[2-(4-methylsulfanylphenyl)phenyl]-4-oxo-1H-quinoline-3-carboxamide |
| 224 | 4-oxo-N-[4-[2-[(2,2,2-trifluoroacetyl)aminomethyl]phenyl]phenyl]-1H-quinoline-3- |
| | carboxamide |
| 225 | [2-isopropyl-5-[(4-oxo-1H-quinolin-3-yl)carbonylamino]phenyl]aminoformic acid ethyl ester |
| 226 | 4-oxo-N-(4-propylphenyl)-1H-quinoline-3-carboxamide |
| 227 | N-[2-(3H-benzoimidazol-2-yl)phenyl]-4-oxo-1H-quinoline-3-carboxamide |
| 228 | N-[2-(hydroxy-phenyl-methyl)phenyl]-4-oxo-1H-quinoline-3-carboxamide |
| 229 | N-(2-methylsulfanylphenyl)-4-oxo-1H-quinoline-3-carboxamide |
| 230 | N-(2-methyl-1H-indol-5-yl)-4-oxo-1H-quinoline-3-carboxamide |
| 231 | 3-[4-hydroxy-2-[(4-oxo-1H-quinolin-3-yl)carbonylamino]-5-tert-butyl-phenyl]benzoic acid |
| | methyl ester |
| 232 | N-(5-acetylamino-2-propyl-phenyl)-4-oxo-1H-quinoline-3-carboxamide |
| 233 | N-(1-acetylindolin-6-yl)-4-oxo-1H-quinoline-3-carboxamide |
| 234 | 4-oxo-N-[5-(trifluoromethyl)-1H-indol-6-yl]-1H-quinoline-3-carboxamide |
| 235 | N-(6-isopropyl-3-pyridyl)-4-oxo-1H-quinoline-3-carboxamide |
| 236 | 4-oxo-N-[4-(trifluoromethyl)phenyl]-1H-quinoline-3-carboxamide |
| 237 | N-[5-(2-methoxyphenyl)-1H-indol-6-yl]-4-oxo-1H-quinoline-3-carboxamide |
| 238 | 7′-[(4-oxo-1H-quinolin-3-ylcarbonyl)amino]-spiro[piperidine-4,4′(1′H)-quinoline], 2′,3′- |
| | dihydro-carboxylic acid tert-butyl ester |
| 239 | [4-isopropyl-3-[(4-oxo-1H-quinolin-3-yl)carbonylamino]phenyl]aminoformic acid methyl ester |
| 240 | N-(2-benzyloxyphenyl)-4-oxo-1H-quinoline-3-carboxamide |
| 241 | 4-oxo-N-(8-quinolyl)-1H-quinoline-3-carboxamide |
| 242 | N-(5-amino-2,4-dichloro-phenyl)-4-oxo-1H-quinoline-3-carboxamide |
| 243 | N-(5-acetylamino-2-isopropyl-phenyl)-4-oxo-1H-quinoline-3-carboxamide |
| 244 | 4-oxo-N-(6,7,8,9-tetrahydro-5H-carbazol-2-yl)-1H-quinoline-3-carboxamide |
| 245 | N-[2-(2,4-dichlorophenoxy)phenyl]-4-oxo-1H-quinoline-3-carboxamide |
| 246 | N-(3,4-dimethylphenyl)-4-oxo-1H-quinoline-3-carboxamide |
| 247 | 4-oxo-N-[2-(2-phenoxyphenyl)phenyl]-1H-quinoline-3-carboxamide |
| 248 | N-(3-acetylamino-4-methyl-phenyl)-4-oxo-1H-quinoline-3-carboxamide |
| 249 | [4-ethyl-3-[(4-oxo-1H-quinolin-3-yl)carbonylamino]phenyl]aminoformic acid methyl ester |
| 250 | N-(5-acetylamino-2-methoxy-phenyl)-4-oxo-1H-quinoline-3-carboxamide |
| 251 | [2-methyl-2-[4-[(4-oxo-1H-quinolin-3-yl)carbonylamino]phenyl]-propyl]aminoformic acid |
| | isobutyl ester |
| 252 | N-(2-benzoylphenyl)-4-oxo-1H-quinoline-3-carboxamide |
| 253 | 4-oxo-N-[2-[3-(trifluoromethoxy)phenyl]phenyl]-1H-quinoline-3-carboxamide |
| 254 | 6-fluoro-N-(5-fluoro-1H-indol-6-yl)-4-oxo-1H-quinoline-3-carboxamide |
| 255 | N-(5-hydroxy-2,4-ditert-butyl-phenyl)-4-oxo-6-pyrrolidin-1-ylsulfonyl-1H-quinoline-3- |
| | carboxamide |
| 256 | N-(1H-benzotriazol-5-yl)-4-oxo-1H-quinoline-3-carboxamide |
| 257 | N-(4-fluoro-3-methyl-phenyl)-4-oxo-1H-quinoline-3-carboxamide |
| 258 | N-indolin-6-yl-4-oxo-1H-quinoline-3-carboxamide |
| 259 | 4-oxo-N-(3-sec-butyl-1H-indol-6-yl)-1H-quinoline-3-carboxamide |
| 260 | N-(5-amino-2-tert-butyl-phenyl)-4-oxo-1H-quinoline-3-carboxamide |
| 261 | N-[2-(3,4-dimethylphenyl)phenyl]-4-oxo-1H-quinoline-3-carboxamide |
| 262 | 1,4-dihydro-N-(3,4-dihydro-3-oxo-2H-benzo[b][1,4]thiazin-6-yl)-4-oxoquinoline-3- |
| | carboxamide |
| 263 | N-(4-bromo-2-ethyl-phenyl)-4-oxo-1H-quinoline-3-carboxamide |
| 264 | N-(2,5-diethoxyphenyl)-4-oxo-1H-quinoline-3-carboxamide |
| 265 | N-(2-benzylphenyl)-4-oxo-1H-quinoline-3-carboxamide |
| 266 | N-[5-hydroxy-4-tert-butyl-2-(trifluoromethyl)phenyl]-4-oxo-1H-quinoline-3-carboxamide |
| 267 | 4-oxo-N-(4-phenoxyphenyl)-1H-quinoline-3-carboxamide |
| 268 | 4-oxo-N-(3-sulfamoyl-4-tert-butyl-phenyl)-1H-quinoline-3-carboxamide |
| 269 | [4-isopropyl-3-[(4-oxo-1H-quinolin-3-yl)carbonylamino]phenyl]aminoformic acid ethyl ester |
| 270 | N-(2-cyano-1H-indol-6-yl)-4-oxo-1H-quinoline-3-carboxamide |
| 271 | N-(3-amino-4-tert-butyl-phenyl)-4-oxo-1H-quinoline-3-carboxamide |
| 272 | N-[3-(2-morpholinoethylsulfonylamino)-5-(trifluoromethyl)phenyl]-4-oxo-1H-quinoline-3- |
| | carboxamide |
| 273 | [7-[(4-oxo-1H-quinolin-3-yl)carbonylamino]tetralin-1-yl]aminoformic acid tert-butyl ester |
| 274 | 4-oxo-6-pyrrolidin-1-ylsulfonyl-N-(5-tert-butyl-1H-indol-6-yl)-1H-quinoline-3-carboxamide |
| 275 | 4-benzyloxy-N-(3-hydroxy-4-tert-butyl-phenyl)-quinoline-3-carboxamide |
| 276 | N-(4-morpholinosulfonylphenyl)-4-oxo-1H-quinoline-3-carboxamide |
| 277 | N-[2-(3-fluorophenyl)phenyl]-4-oxo-1H-quinoline-3-carboxamide |
| 278 | 4-oxo-N-[2-[3-(trifluoromethyl)phenyl]phenyl]-1H-quinoline-3-carboxamide |
| 279 | N-[2-(2-methylsulfanylphenyl)phenyl]-4-oxo-1H-quinoline-3-carboxamide |
| 280 | 4-oxo-N-(6-quinolyl)-1H-quinoline-3-carboxamide |
| 281 | N-(2,4-dimethylphenyl)-4-oxo-1H-quinoline-3-carboxamide |
| 282 | N-(5-amino-2-ethyl-phenyl)-4-oxo-1H-quinoline-3-carboxamide |
| 283 | N-[2-(3-methoxyphenyl)phenyl]-4-oxo-1H-quinoline-3-carboxamide |
| 284 | N-(1H-indazol-6-yl)-4-oxo-1H-quinoline-3-carboxamide |
| 285 | N-[2-(2,3-difluorophenyl)phenyl]-4-oxo-1H-quinoline-3-carboxamide |
| 286 | 1,4-dihydro-N-(1,2,3,4-tetrahydronaphthalen-5-yl)-4-oxoquinoline-3-carboxamide |
| 287 | N-[2-fluoro-5-hydroxy-4-(1-methylcyclohexyl)-phenyl]-5-hydroxy-4-oxo-1H-quinoline-3- |
| | carboxamide |
| 288 | N-(5-fluoro-2-methoxycarbonyloxy-3-tert-butyl-phenyl)-4-oxo-1H-quinoline-3-carboxamide |
| 289 | N-(2-fluoro-4-methyl-phenyl)-4-oxo-1H-quinoline-3-carboxamide |
| 290 | N-[2-(3-isopropylphenyl)phenyl]-4-oxo-1H-quinoline-3-carboxamide |
| 291 | N-(2-chloro-5-hydroxy-4-tert-butyl-phenyl)-4-oxo-1H-quinoline-3-carboxamide |
| 292 | N-(5-chloro-2-phenoxy-phenyl)-4-oxo-1H-quinoline-3-carboxamide |
| 293 | 4-oxo-N-[2-(1H-pyrrol-1-yl)phenyl]-1H-quinoline-3-carboxamide |
| 294 | N-(1H-indol-5-yl)-4-oxo-1H-quinoline-3-carboxamide |
| 295 | 4-oxo-N-(2-pyrrolidin-1-ylphenyl)-1H-quinoline-3-carboxamide |
| 296 | 2,4-dimethoxy-N-(2-tert-butylphenyl)-quinoline-3-carboxamide |
| 297 | N-[2-(2,5-dimethyl-1H-pyrrol-1-yl)phenyl]-4-oxo-1H-quinoline-3-carboxamide |
| 298 | [2-ethyl-5-[(4-oxo-1H-quinolin-3-yl)carbonylamino]phenyl]aminoformic acid ethyl ester |
| 299 | 4-oxo-N-(1,2,3,4-tetrahydroquinolin-7-yl)-1H-quinoline-3-carboxamide |
| 300 | N-(4,4-dimethyl-1,2,3,4-tetrahydroquinolin-7-yl)-4-oxo-1H-quinoline-3-carboxamide |
| 301 | N-[4-(4-methyl-4H-1,2,4-triazol-3-yl)phenyl]-4-oxo-1H-quinoline-3-carboxamide |
| 302 | N-[2-[4-(hydroxymethyl)phenyl]phenyl]-4-oxo-1H-quinoline-3-carboxamide |
| 303 | N-(2-acetyl-1,2,3,4-tetrahydroisoquinolin-7-yl)-4-oxo-1H-quinoline-3-carboxamide |
| 304 | [4-(2-ethoxyphenyl)-3-[(4-oxo-1H-quinolin-3-yl)carbonylamino]phenylmethyl]aminoformic |
| | acid tert-butyl ester |
| 305 | N-[2-(4-methoxyphenyl)phenyl]-4-oxo-1H-quinoline-3-carboxamide |
| 306 | N-[2-(3-ethoxyphenyl)phenyl]-4-oxo-1H-quinoline-3-carboxamide |
| 307 | N-[2-(3-chlorophenyl)phenyl]-4-oxo-1H-quinoline-3-carboxamide |
| 308 | N-[2-(cyanomethyl)phenyl]-4-oxo-1H-quinoline-3-carboxamide |
| 309 | N-(3-isoquinolyl)-4-oxo-1H-quinoline-3-carboxamide |
| 310 | 4-oxo-N-(4-sec-butylphenyl)-1H-quinoline-3-carboxamide |
| 311 | N-[2-(5-methyl-2-furyl)phenyl]-4-oxo-1H-quinoline-3-carboxamide |
| 312 | N-[2-(2,4-dimethoxyphenyl)phenyl]-4-oxo-1H-quinoline-3-carboxamide |
| 313 | N-[2-(2-fluorophenyl)phenyl]-4-oxo-1H-quinoline-3-carboxamide |
| 314 | N-(2-ethyl-6-isopropyl-phenyl)-4-oxo-1H-quinoline-3-carboxamide |
| 315 | N-(2,6-dimethylphenyl)-4-oxo-1H-quinoline-3-carboxamide |
| 316 | N-(5-acetylamino-2-tert-butyl-phenyl)-4-oxo-1H-quinoline-3-carboxamide |
| 317 | N-(2,6-dichlorophenyl)-4-oxo-1H-quinoline-3-carboxamide |
| 318 | 4-oxo-N-[3-[2-(1-piperidyl)ethylsulfonylamino]-5-(trifluoromethyl)phenyl]-1H-quinoline-3- |
| | carboxamide |
| 319 | 6-fluoro-N-(2-fluoro-5-hydroxy-4-tert-butyl-phenyl)-4-oxo-1H-quinoline-3-carboxamide |
| 320 | 4-oxo-N-(2-tert-butyl-1H-indol-6-yl)-1H-quinoline-3-carboxamide |
| 321 | N-[2-(4-benzoylpiperazin-1-yl)phenyl]-4-oxo-1H-quinoline-3-carboxamide |
| 322 | N-(2-ethyl-6-sec-butyl-phenyl)-4-oxo-1H-quinoline-3-carboxamide |
| 323 | [2-methyl-2-[4-[(4-oxo-1H-quinolin-3-yl)carbonylamino]phenyl]-propyl]aminoformic acid |
| | methyl ester |
| 324 | N-(4-butylphenyl)-4-oxo-1H-quinoline-3-carboxamide |
| 325 | N-(2,6-diethylphenyl)-4-oxo-1H-quinoline-3-carboxamide |
| 326 | N-[2-(4-methylsulfonylphenyl)phenyl]-4-oxo-1H-quinoline-3-carboxamide |
| 327 | N-[5-(2-ethoxyphenyl)-1H-indol-6-yl]-4-oxo-1H-quinoline-3-carboxamide |
| 328 | N-(3-acetylphenyl)-4-oxo-1H-quinoline-3-carboxamide |
| 329 | N-[2-(o-tolyl)benzooxazol-5-yl]-4-oxo-1H-quinoline-3-carboxamide |
| 330 | N-(2-chlorophenyl)-4-oxo-1H-quinoline-3-carboxamide |
| 331 | N-(2-carbamoylphenyl)-4-oxo-1H-quinoline-3-carboxamide |
| 332 | N-(4-ethynylphenyl)-4-oxo-1H-quinoline-3-carboxamide |
| 333 | N-[2-[4-(cyanomethyl)phenyl]phenyl]-4-oxo-1H-quinoline-3-carboxamide |
| 334 | 7′-[(4-oxo-1H-quinolin-3-ylcarbonyl)amino]-spiro[piperidine-4,4′(1′H)-1-acetyl-quinoline], |
| | 2′,3′-dihydro-carboxylic acid tert-butyl ester |
| 335 | N-(2-carbamoyl-5-methyl-phenyl)-4-oxo-1H-quinoline-3-carboxamide |
| 336 | N-(2-butylphenyl)-4-oxo-1H-quinoline-3-carboxamide |
| 337 | N-(5-hydroxy-2,4-ditert-butyl-phenyl)-N-methyl-4-oxo-1H-quinoline-3-carboxamide |
| 338 | N-(3-methyl-1H-indol-4-yl)-4-oxo-1H-quinoline-3-carboxamide |
| 339 | N-(3-cyano-1H-indol-6-yl)-4-oxo-1H-quinoline-3-carboxamide |
| 340 | N-(3-methylsulfonylamino-4-propyl-phenyl)-4-oxo-1H-quinoline-3-carboxamide |
| 341 | [2-methyl-2-[4-[(4-oxo-1H-quinolin-3-yl)carbonylamino]phenyl]-propyl]aminoformic acid |
| | neopentyl ester |
| 342 | N-[5-(4-isopropylphenyl)-1H-indol-6-yl]-4-oxo-1H-quinoline-3-carboxamide |
| 343 | N-[5-(isobutylcarbamoyl)-1H-indol-6-yl]-4-oxo-1H-quinoline-3-carboxamide |
| 344 | N-[2-(2-ethoxyphenyl)phenyl]-4-oxo-1H-quinoline-3-carboxamide |
| 345 | 6-fluoro-4-hydroxy-N-(1H-indol-6-yl)quinoline-3-carboxamide |
| 346 | 4-oxo-N-phenyl-7-(trifluoromethyl)-1H-quinoline-3-carboxamide |
| 347 | N-[5-[4-(2-dimethylaminoethylcarbamoyl)phenyl]-1H-indol-6-yl]-4-oxo-1H-quinoline-3- |
| | carboxamide |
| 348 | N-[2-(4-ethoxyphenyl)phenyl]-4-oxo-1H-quinoline-3-carboxamide |
| 349 | 4-oxo-N-(2-phenylsulfonylphenyl)-1H-quinoline-3-carboxamide |
| 350 | N-(1-naphthyl)-4-oxo-1H-quinoline-3-carboxamide |
| 351 | N-(5-ethyl-1H-indol-6-yl)-4-oxo-1H-quinoline-3-carboxamide |
| 352 | 2-[6-[(4-oxo-1H-quinolin-3-yl)carbonylamino]-1H-indol-3-yl]ethylaminoformic acid tert-butyl |
| | ester |
| 353 | [3-[(4-oxo-1H-quinolin-3-yl)carbonylamino]-4-tert-butyl-phenyl]aminoformic acid tert-butyl |
| | ester |
| 354 | N-[2-[(cyclohexyl-methyl-amino)methyl]phenyl]-4-oxo-1H-quinoline-3-carboxamide |
| 355 | N-[2-(2-methoxyphenyl)phenyl]-4-oxo-1H-quinoline-3-carboxamide |
| 356 | N-(5-methylamino-2-propyl-phenyl)-4-oxo-1H-quinoline-3-carboxamide |
| 357 | N-(3-isopropyl-1H-indol-6-yl)-4-oxo-1H-quinoline-3-carboxamide |
| 358 | 6-chloro-4-hydroxy-N-(1H-indol-6-yl)quinoline-3-carboxamide |
| 359 | N-[3-(2-dimethylaminoethylsulfonylamino)-5-(trifluoromethyl)phenyl]-4-oxo-1H-quinoline-3- |
| | carboxamide |
| 360 | N-[4-(difluoromethoxy)phenyl]-4-oxo-1H-quinoline-3-carboxamide |
| 361 | N-[2-(2,5-dimethoxyphenyl)phenyl]-4-oxo-1H-quinoline-3-carboxamide |
| 362 | N-(2-chloro-4-tert-butyl-phenyl)-4-oxo-1H-quinoline-3-carboxamide |
| 363 | N-[2-(2-fluoro-3-methoxy-phenyl)phenyl]-4-oxo-1H-quinoline-3-carboxamide |
| 364 | N-(2-methyl-8-quinolyl)-4-oxo-1H-quinoline-3-carboxamide |
| 365 | N-(2-acetylphenyl)-4-oxo-1H-quinoline-3-carboxamide |
| 366 | 4-oxo-N-[2-[4-(trifluoromethyl)phenyl]phenyl]-1H-quinoline-3-carboxamide |
| 367 | N-[2-(3,5-dichlorophenyl)phenyl]-4-oxo-1H-quinoline-3-carboxamide |
| 368 | N-(3-amino-4-propoxy-phenyl)-4-oxo-1H-quinoline-3-carboxamide |
| 369 | N-(2,4-dichloro-6-cyano-phenyl)-4-oxo-1H-quinoline-3-carboxamide |
| 370 | N-(3-chlorophenyl)-4-oxo-1H-quinoline-3-carboxamide |
| 371 | 4-oxo-N-[2-(trifluoromethylsulfanyl)phenyl]-1H-quinoline-3-carboxamide |
| 372 | N-[2-(4-methyl-1-piperidyl)phenyl]-4-oxo-1H-quinoline-3-carboxamide |
| 373 | N-indan-4-yl-4-oxo-1H-quinoline-3-carboxamide |
| 374 | 4-hydroxy-N-(1H-indol-6-yl)-2-methylsulfanyl-quinoline-3-carboxamide |
| 375 | 1,4-dihydro-N-(1,2,3,4-tetrahydronaphthalen-6-yl)-4-oxoquinoline-3-carboxamide |
| 376 | 4-oxo-N-(2-phenylbenzooxazol-5-yl)-1H-quinoline-3-carboxamide |
| 377 | 6,8-difluoro-4-hydroxy-N-(1H-indol-6-yl)quinoline-3-carboxamide |
| 378 | N-(3-amino-4-methoxy-phenyl)-4-oxo-1H-quinoline-3-carboxamide |
| 379 | N-[3-acetylamino-5-(trifluoromethyl)phenyl]-4-oxo-1H-quinoline-3-carboxamide |
| 380 | N-(2-ethoxyphenyl)-4-oxo-1H-quinoline-3-carboxamide |
| 381 | 4-oxo-N-(5-tert-butyl-1H-indol-6-yl)-1H-quinoline-3-carboxamide |
| 382 | [5-[(4-oxo-1H-quinolin-3-yl)carbonylamino]-2-propyl-phenyl]aminoformic acid ethyl ester |
| 383 | N-(3-ethyl-1H-indol-6-yl)-4-oxo-1H-quinoline-3-carboxamide |
| 384 | N-[2-(2,5-difluorophenyl)phenyl]-4-oxo-1H-quinoline-3-carboxamide |
| 385 | N-[2-(2,4-difluorophenoxy)-3-pyridyl]-4-oxo-1H-quinoline-3-carboxamide |
| 386 | N-(3,3-dimethylindolin-6-yl)-4-oxo-1H-quinoline-3-carboxamide |
| 387 | N-[2-methyl-3-(trifluoromethyl)phenyl]-4-oxo-1H-quinoline-3-carboxamide |
| 388 | 4-oxo-N-[2-[4-(trifluoromethoxy)phenyl]phenyl]-1H-quinoline-3-carboxamide |
| 389 | N-(3-benzylphenyl)-4-oxo-1H-quinoline-3-carboxamide |
| 390 | N-[3-(aminomethyl)-4-tert-butyl-phenyl]-4-oxo-1H-quinoline-3-carboxamide |
| 391 | N-[2-(4-isobutylphenyl)phenyl]-4-oxo-1H-quinoline-3-carboxamide |
| 392 | N-(6-chloro-3-pyridyl)-4-oxo-1H-quinoline-3-carboxamide |
| 393 | N-[5-amino-2-(2-ethoxyphenyl)-phenyl]-4-oxo-1H-quinoline-3-carboxamide |
| 394 | 1,6-dimethyl-4-oxo-N-phenyl-1H-quinoline-3-carboxamide |
| 395 | N-[4-(1-adamantyl)-2-fluoro-5-hydroxy-phenyl]-4-hydroxy-quinoline-3-carboxamide |
| 396 | [2-methyl-2-[4-[(4-oxo-1H-quinolin-3-yl)carbonylamino]phenyl]-propyl]aminoformic acid |
| | tetrahydrofuran-3-ylmethyl ester |
| 397 | 4-oxo-N-(4-phenylphenyl)-1H-quinoline-3-carboxamide |
| 398 | 4-oxo-N-[2-(p-tolylsulfonylamino)phenyl]-1H-quinoline-3-carboxamide |
| 399 | N-(2-isopropyl-5-methylamino-phenyl)-4-oxo-1H-quinoline-3-carboxamide |
| 400 | N-(6-morpholino-3-pyridyl)-4-oxo-1H-quinoline-3-carboxamide |
| 401 | N-[2-(2,3-dimethylphenyl)phenyl]-4-oxo-1H-quinoline-3-carboxamide |
| 402 | 4-oxo-N-(5-phenyl-2-pyridyl)-1H-quinoline-3-carboxamide |
| 403 | N-[2-fluoro-5-hydroxy-4-(1-methylcyclooctyl)-phenyl]-4-hydroxy-quinoline-3-carboxamide |
| 404 | N-[5-(2,6-dimethoxyphenyl)-1H-indol-6-yl]-4-oxo-1H-quinoline-3-carboxamide |
| 405 | N-(4-chlorophenyl)-4-oxo-1H-quinoline-3-carboxamide |
| 406 | 6-[(4-fluorophenyl)-methyl-sulfamoyl]-4-oxo-N-(5-tert-butyl-1H-indol-6-yl)-1H-quinoline-3- |
| | carboxamide |
| 407 | N-(2-fluoro-5-hydroxy-4-tert-butyl-phenyl)-5-hydroxy-4-oxo-1H-quinoline-3-carboxamide |
| 408 | N-(3-methoxyphenyl)-4-oxo-1H-quinoline-3-carboxamide |
| 409 | N-(5-dimethylamino-2-ethyl-phenyl)-4-oxo-1H-quinoline-3-carboxamide |
| 410 | 4-oxo-N-[2-(4-phenoxyphenyl)phenyl]-1H-quinoline-3-carboxamide |
| 411 | 7-chloro-4-oxo-N-phenyl-1H-quinoline-3-carboxamide |
| 412 | 6-[(4-oxo-1H-quinolin-3-yl)carbonylamino]-1H-indole-7-carboxylic acid ethyl ester |
| 413 | 4-oxo-N-(2-phenoxyphenyl)-1H-quinoline-3-carboxamide |
| 414 | N-(3H-benzoimidazol-5-yl)-4-oxo-1H-quinoline-3-carboxamide |
| 415 | N-(3-hydroxy-4-tert-butyl-phenyl)-4-methoxy-quinoline-3-carboxamide |
| 416 | [2-methyl-2-[4-[(4-oxo-1H-quinolin-3-yl)carbonylamino]phenyl]-propyl]aminoformic acid |
| | propyl ester |
| 417 | N-(2-(benzo[b]thiophen-3-yl)phenyl)-1,4-dihydro-4-oxoquinoline-3-carboxamide |
| 418 | N-(3-dimethylaminophenyl)-4-oxo-1H-quinoline-3-carboxamide |
| 419 | N-(3-acetylaminophenyl)-4-oxo-1H-quinoline-3-carboxamide |
| 420 | 2-methyl-2-[4-[(4-oxo-1H-quinolin-3-yl)carbonylamino]phenyl]-propanoic acid ethyl ester |
| 421 | N-[5-methoxy-4-tert-butyl-2-(trifluoromethyl)phenyl]-4-oxo-1H-quinoline-3-carboxamide |
| 422 | N-(5,6-dimethyl-3H-benzoimidazol-2-yl)-4-oxo-1H-quinoline-3-carboxamide |
| 423 | N-[3-(2-ethoxyethyl)-1H-indol-6-yl]-4-oxo-1H-quinoline-3-carboxamide |
| 424 | N-[2-(4-chlorophenyl)phenyl]-4-oxo-1H-quinoline-3-carboxamide |
| 425 | N-(4-isopropylphenyl)-4-oxo-1H-quinoline-3-carboxamide |
| 426 | N-(4-chloro-5-hydroxy-2-tert-butyl-phenyl)-4-oxo-1H-quinoline-3-carboxamide |
| 427 | 5-[(4-oxo-1H-quinolin-3-yl)carbonylamino]-1,2,3,4-tetrahydroisoquinoline-2-carboxylic acid |
| | tert-butyl ester |
| 428 | N-(3-hydroxy-4-tert-butyl-phenyl)-4-oxo-1H-quinoline-3-carboxamide |
| 429 | N-[3-amino-5-(trifluoromethyl)phenyl]-4-oxo-1H-quinoline-3-carboxamide |
| 430 | N-(2-isopropyl-6-methyl-phenyl)-4-oxo-1H-quinoline-3-carboxamide |
| 431 | N-(3-aminophenyl)-4-oxo-1H-quinoline-3-carboxamide |
| 432 | N-[2-(4-isopropylphenyl)phenyl]-4-oxo-1H-quinoline-3-carboxamide |
| 433 | N-(5-hydroxy-2,4-ditert-butyl-phenyl)-4-oxo-1H-quinoline-3-carboxamide |
| 434 | N-(2,5-dimethylphenyl)-4-oxo-1H-quinoline-3-carboxamide |
| 435 | N-[2-(2-fluorophenoxy)-3-pyridyl]-4-oxo-1H-quinoline-3-carboxamide |
| 436 | N-[2-(3,4-dimethoxyphenyl)phenyl]-4-oxo-1H-quinoline-3-carboxamide |
| 437 | N-benzo[1,3]dioxol-5-yl-4-oxo-1H-quinoline-3-carboxamide |
| 438 | N-[5-(difluoromethyl)-2,4-ditert-butyl-phenyl]-4-oxo-1H-quinoline-3-carboxamide |
| 439 | N-(4-methoxyphenyl)-4-oxo-1H-quinoline-3-carboxamide |
| 440 | N-(2,2,3,3-tetrafluoro-2,3-dihydrobenzo[b][1,4]dioxin-6-yl)-1,4-dihydro-4-oxoquinoline-3- |
| | carboxamide |
| 441 | N-[3-methylsulfonylamino-5-(trifluoromethyl)phenyl]-4-oxo-1H-quinoline-3-carboxamide |
| 442 | 4-oxo-N-[3-(1-piperidylsulfonyl)phenyl]-1H-quinoline-3-carboxamide |
| 443 | 4-oxo-N-quinoxalin-6-yl-1H-quinoline-3-carboxamide |
| 444 | 5-[(4-oxo-1H-quinolin-3-yl)carbonylamino]-2-tert-butyl-benzoic acid methyl ester |
| 445 | N-(2-isopropenylphenyl)-4-oxo-1H-quinoline-3-carboxamide |
| 446 | N-(1,1-dioxobenzothiophen-6-yl)-4-oxo-1H-quinoline-3-carboxamide |
| 447 | N-(3-cyanophenyl)-4-oxo-1H-quinoline-3-carboxamide |
| 448 | 4-oxo-N-(4-tert-butylphenyl)-1H-quinoline-3-carboxamide |
| 449 | N-(m-tolyl)-4-oxo-1H-quinoline-3-carboxamide |
| 450 | N-[4-(1-hydroxyethyl)phenyl]-4-oxo-1H-quinoline-3-carboxamide |
| 451 | N-(4-cyano-2-ethyl-phenyl)-4-oxo-1H-quinoline-3-carboxamide |
| 452 | 4-oxo-N-(4-vinylphenyl)-1H-quinoline-3-carboxamide |
| 453 | N-(3-amino-4-chloro-phenyl)-4-oxo-1H-quinoline-3-carboxamide |
| 454 | N-(2-methyl-5-phenyl-phenyl)-4-oxo-1H-quinoline-3-carboxamide |
| 455 | N-[4-(1-adamantyl)phenyl]-4-oxo-1H-quinoline-3-carboxamide |
| 456 | 4-oxo-N-[3-(trifluoromethylsulfanyl)phenyl]-1H-quinoline-3-carboxamide |
| 457 | N-(4-morpholinophenyl)-4-oxo-1H-quinoline-3-carboxamide |
| 458 | N-[3-(2-hydroxyethoxy)-4-tert-butyl-phenyl]-4-oxo-1H-quinoline-3-carboxamide |
| 459 | N-(o-tolyl)-4-oxo-1H-quinoline-3-carboxamide |
| 460 | [2-methyl-2-[4-[(4-oxo-1H-quinolin-3-yl)carbonylamino]phenyl]-propyl]aminoformic acid |
| | butyl ester |
| 461 | 4-oxo-N-(2-phenylphenyl)-1H-quinoline-3-carboxamide |
| 462 | N-(3-dimethylamino-4-propyl-phenyl)-4-oxo-1H-quinoline-3-carboxamide |
| 463 | N-(4-ethylphenyl)-4-oxo-1H-quinoline-3-carboxamide |
| 464 | 5-hydroxy-N-(5-hydroxy-2,4-ditert-butyl-phenyl)-4-oxo-1H-quinoline-3-carboxamide |
| 465 | [5-[(4-oxo-1H-quinolin-3-yl)carbonylamino]-2-tert-butyl-phenylmethyl]aminoformic acid tert- |
| | butyl ester |
| 466 | N-(2,6-diisopropylphenyl)-4-oxo-1H-quinoline-3-carboxamide |
| 467 | N-(2,3-dihydrobenzofuran-5-yl)-4-oxo-1H-quinoline-3-carboxamide |
| 468 | 1-methyl-4-oxo-N-phenyl-1H-quinoline-3-carboxamide |
| 469 | 4-oxo-N-(2-phenylphenyl)-7-(trifluoromethyl)-1H-quinoline-3-carboxamide |
| 470 | 4-oxo-N-(4-phenylsulfanylphenyl)-1H-quinoline-3-carboxamide |
| 471 | [3-[(4-oxo-1H-quinolin-3-yl)carbonylamino]-4-propyl-phenyl]aminoformic acid methyl ester |
| 472 | [4-ethyl-3-[(4-oxo-1H-quinolin-3-yl)carbonylamino]phenyl]aminoformic acid ethyl ester |
| 473 | 1-isopropyl-4-oxo-N-(2-tert-butylphenyl)-1H-quinoline-3-carboxamide |
| 474 | N-(3-methyl-2-oxo-3H-benzooxazol-5-yl)-4-oxo-1H-quinoline-3-carboxamide |
| 475 | N-(2,5-dichloro-3-pyridyl)-4-oxo-1H-quinoline-3-carboxamide |
| 476 | N-(2-cyano-5-hydroxy-4-tert-butyl-phenyl)-4-oxo-1H-quinoline-3-carboxamide |
| 477 | N-(5-fluoro-2-pyridyl)-4-oxo-1H-quinoline-3-carboxamide |
| 478 | 4-oxo-N-(3-tert-butyl-1H-indol-6-yl)-1H-quinoline-3-carboxamide |
| 479 | N-(1H-indol-6-yl)-5-methoxy-4-oxo-1H-quinoline-3-carboxamide |
| 480 | 1-ethyl-6-methoxy-4-oxo-N-phenyl-1H-quinoline-3-carboxamide |
| 481 | N-(2-naphthyl)-4-oxo-1H-quinoline-3-carboxamide |
| 482 | [7-[(4-oxo-1H-quinolin-3-yl)carbonylamino]tetralin-1-yl]aminoformic acid ethyl ester |
| 483 | N-[2-fluoro-5-hydroxy-4-(1-methylcycloheptyl)-phenyl]-4-hydroxy-quinoline-3-carboxamide |
| 484 | N-(3-methylamino-4-tert-butyl-phenyl)-4-oxo-1H-quinoline-3-carboxamide |
| 485 | N-(3-dimethylamino-4-tert-butyl-phenyl)-4-oxo-1H-quinoline-3-carboxamide |

Synthesis of Acid Precursors P-IV-A, P-IV-B or P-IV-C:

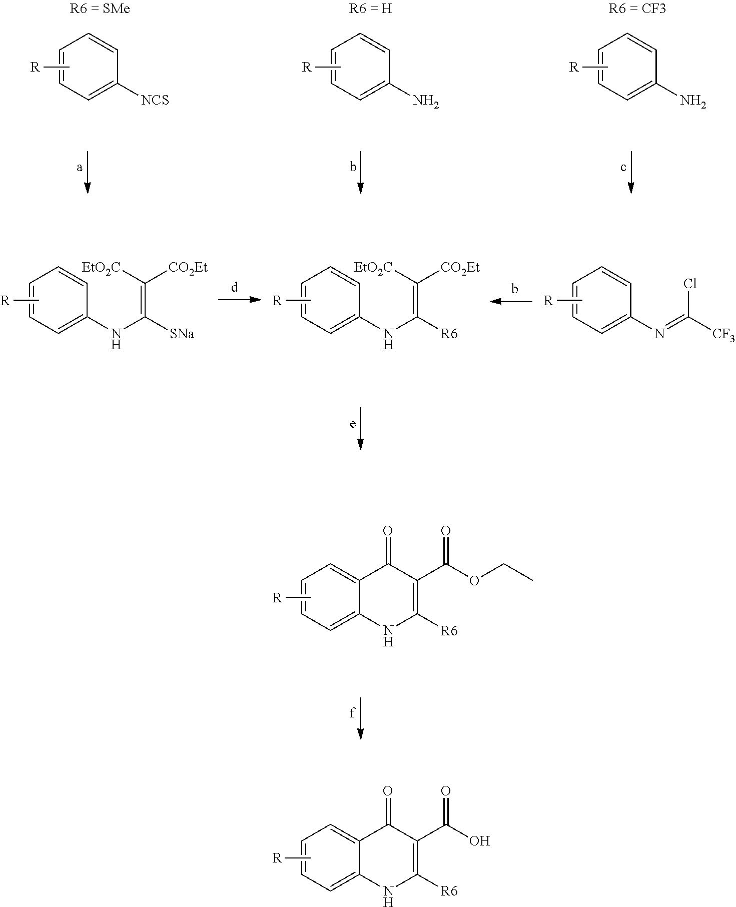

a) (CO₂Et)₂CH₂; b) (CO₂Et)₂CH═CH(OEt); c) CF₃CO₂H, PPh₃, CCl₄, Et₃N; d) MeI; e) PPA or diphenylether; f) NaOH.

Synthesis of Acid Precursors P-IV-A, P-IV-B or P-IV-C:

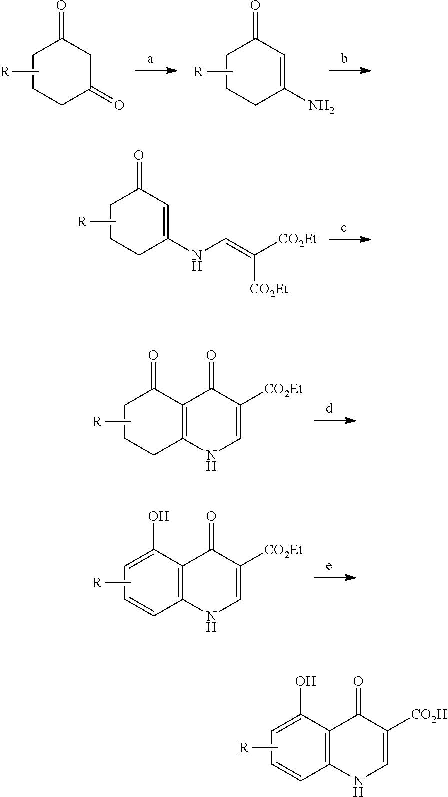

a) AcONH₄; b) EtOCHC(CO₂Et)₂, 130° C.; c) Ph₂O, T; d) I₂, EtOH; e) NaOH.

Synthesis of Acid Precursors P-IV-A, P-IV-B or P-IV-C

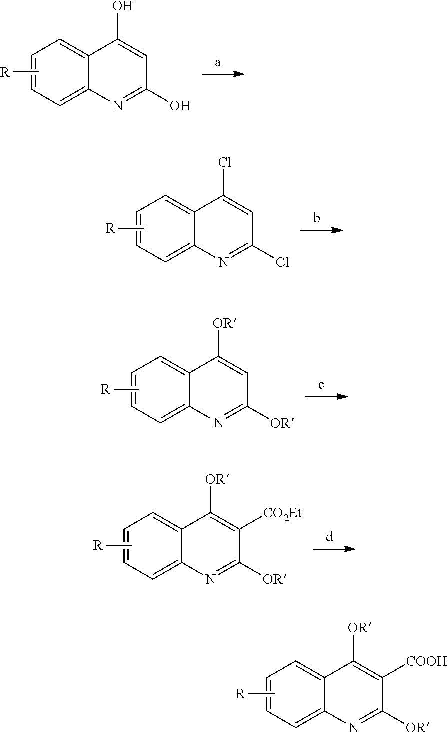

POCl₃; b) R′ONa; c) n-BuLi, ClCO₂Et; d) NaOH

Synthesis of Amine Precursor P-III-A:

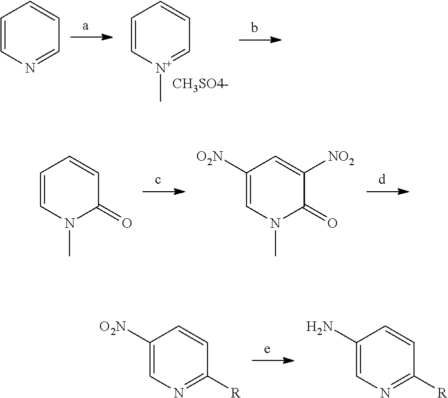

(CH₃)₂SO₄; b) K₃Fe(CN)₆, NaOH, H₂O; c) HNO₃, H₂SO₄; d) RCOCH₃, MeOH, NH₃; e) H₂, Raney Ni

Synthesis of Amine Precursor P-IV-A:

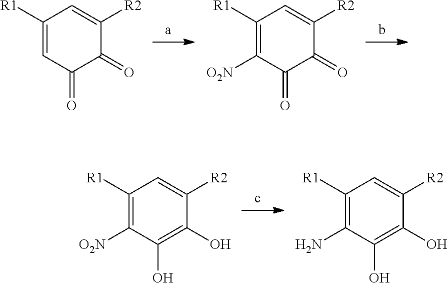

HNO₃, HOAc; b) Na₂S₂O₄, THF/H₂O; c) H₂, Pd/C.

Synthesis of Amine Precursor P-V-A-1:

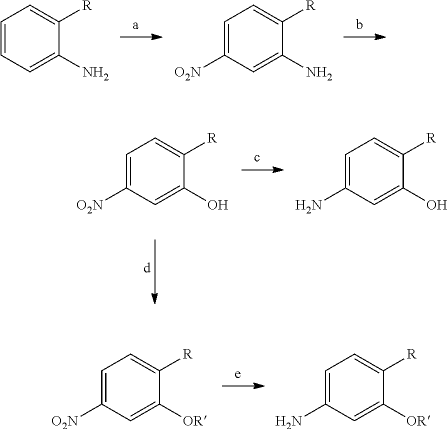

KNO₃, H₂SO₄; b) NaNO₂, H₂SO₄—H₂O; c) NH₄CO₂H, Pd—C; d) R′X; e) NH₄CO₂H, Pd—C

Synthesis of Amine Precursor P-V-A-1:

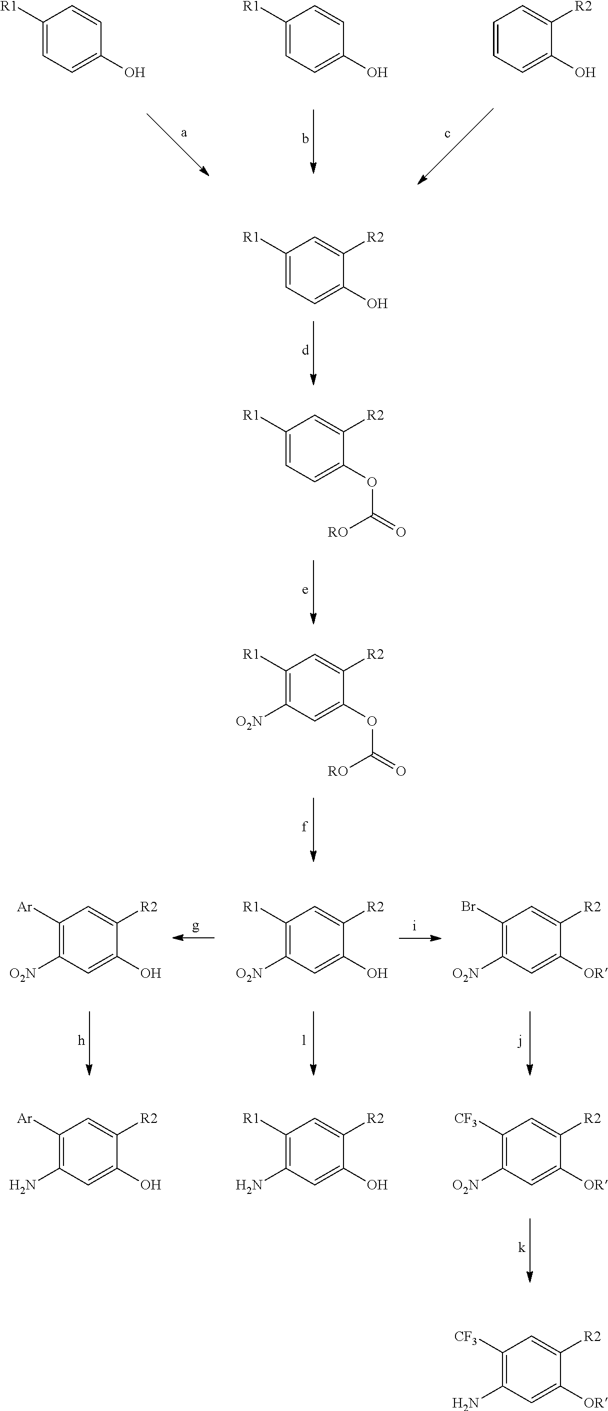

b) SO₂Cl₂, R2=Cl; b) R2OH, R2=alkyl; c) NBS, R1=Br; d) ClCO₂R, TEA; e) HNO₃, H₂SO₄; f) base; g) ArB(OH)₂, R1=Br; h) [H]; I) R′X, R1=Br; j) ClCF₂CO₂Me; k) [H]; l) [H].

Synthesis of Amine Precursor P-V-A-1:

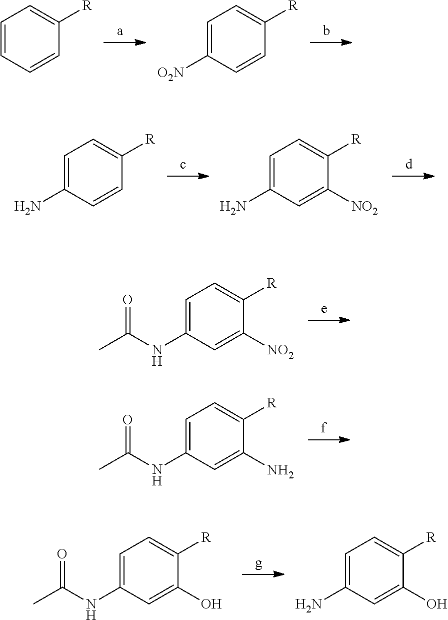

KNO₃; b) [H]; c) KNO₃; d) AcCl; e) [H]; f) i) NaNO₂; ii) H₂O; g) HCl

Synthesis of Amine Precursor P-V-A-1:

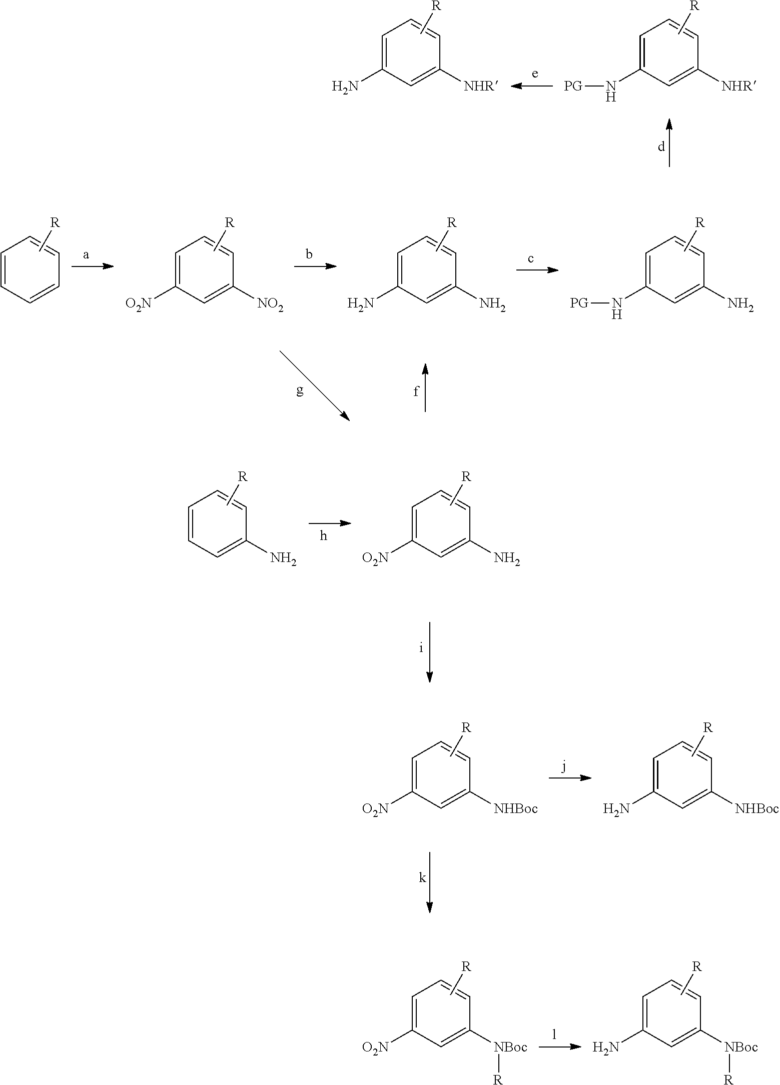

HNO₃, H₂SO₄; b) [H]; c) protection; d) R′CHO; e) deprotection; f) [H]; g) Na₂S, S, H₂O; h) nitration; i) (BOC)₂O; j) [H]; k) RX; l) [H]; PG=protecting group

Synthesis of Amine Precursors P-V-A-1 or P-V-A-2:

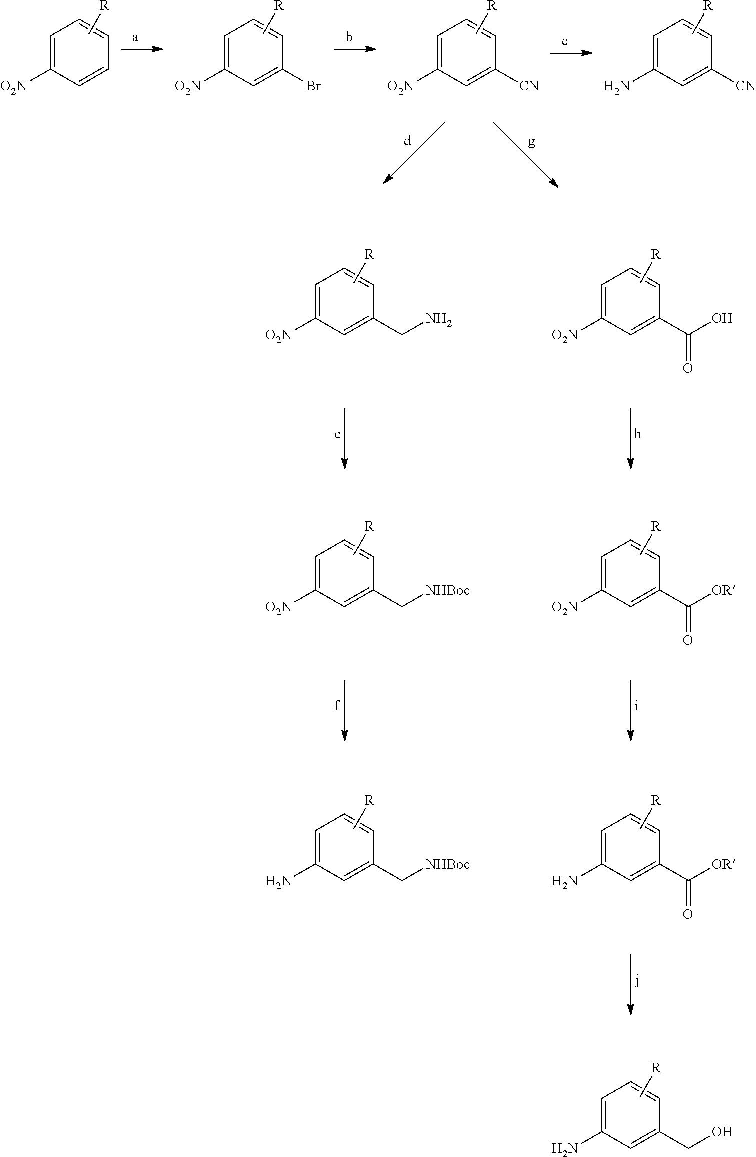

a) Br₂; b) Zn(CN)₂, Pd(PPh)₃; c) [H]; d) BH₃; e) (BOC)₂O; f) [H]; g) H₂SO₄, H₂O; h) R′X; i) [H]; j) LiAlH₄

Synthesis of Amine Precursors P-V-A-1 or P-V-A-2:

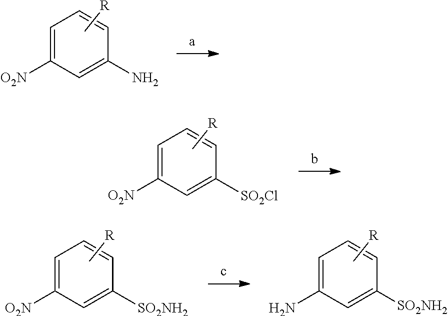

(i)NaNO₂, HCl; ii) Na₂SO₃, CuSO₄, HCl; b) NH₄Cl; c) [H]

Synthesis of Amine Precursors P-V-A-1:

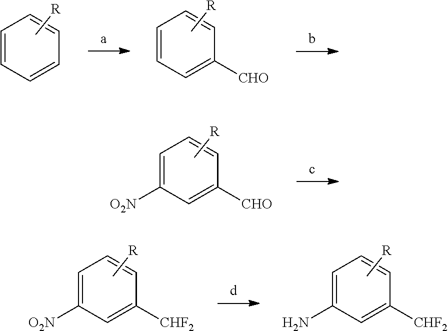

a) CHCl₂OMe; b) KNO₃, H₂SO₄; c) Deoxo-Fluor; d) Fe

Synthesis of Amine Precursors P-V-A-3:

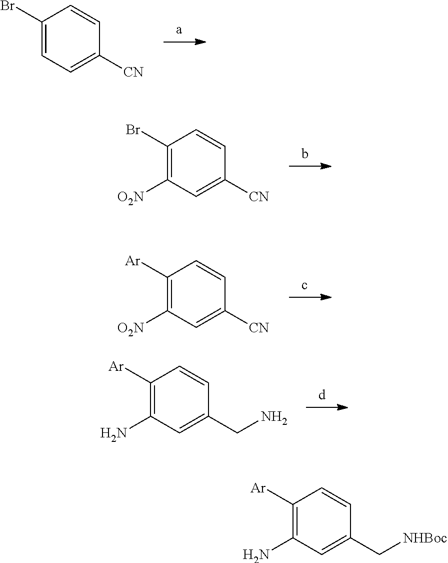

### Ar=Aryl or Heteroaryl

### a) Nitration; b) ArB(OH)₂, Pd; c) BH₃; d) (BOC)₂O

Synthesis of Amine Precursors P-V-B-1:

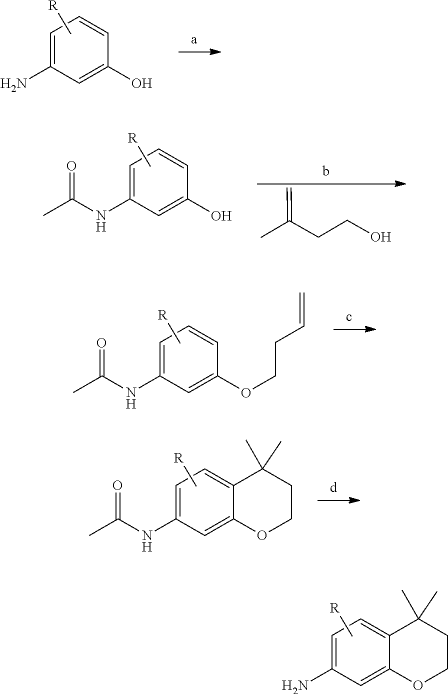

### a) AcCl; b) DEAD; c) AlCl₃; d) NaOH

Synthesis of Amine Precursors P-V-B-1:

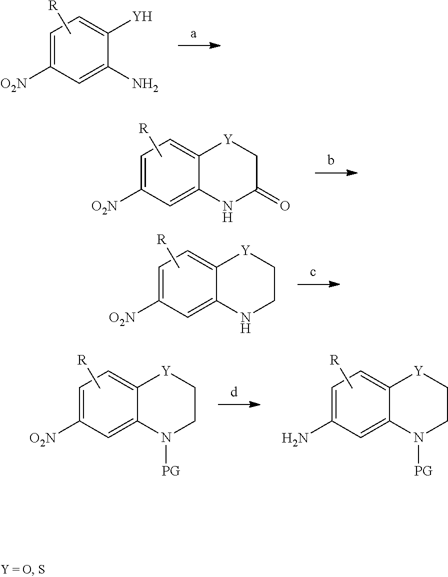

a) ClCH₂COCl; b) [H]; c) protection; d) [H]
PG=protecting group

Synthesis of Amine Precursors P-V-B-1:

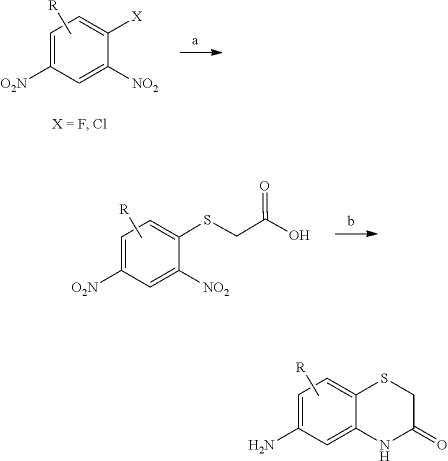

### a) HSCH₂CO₂H; b) [H]

Synthesis of Amine Precursors P-V-B-2:

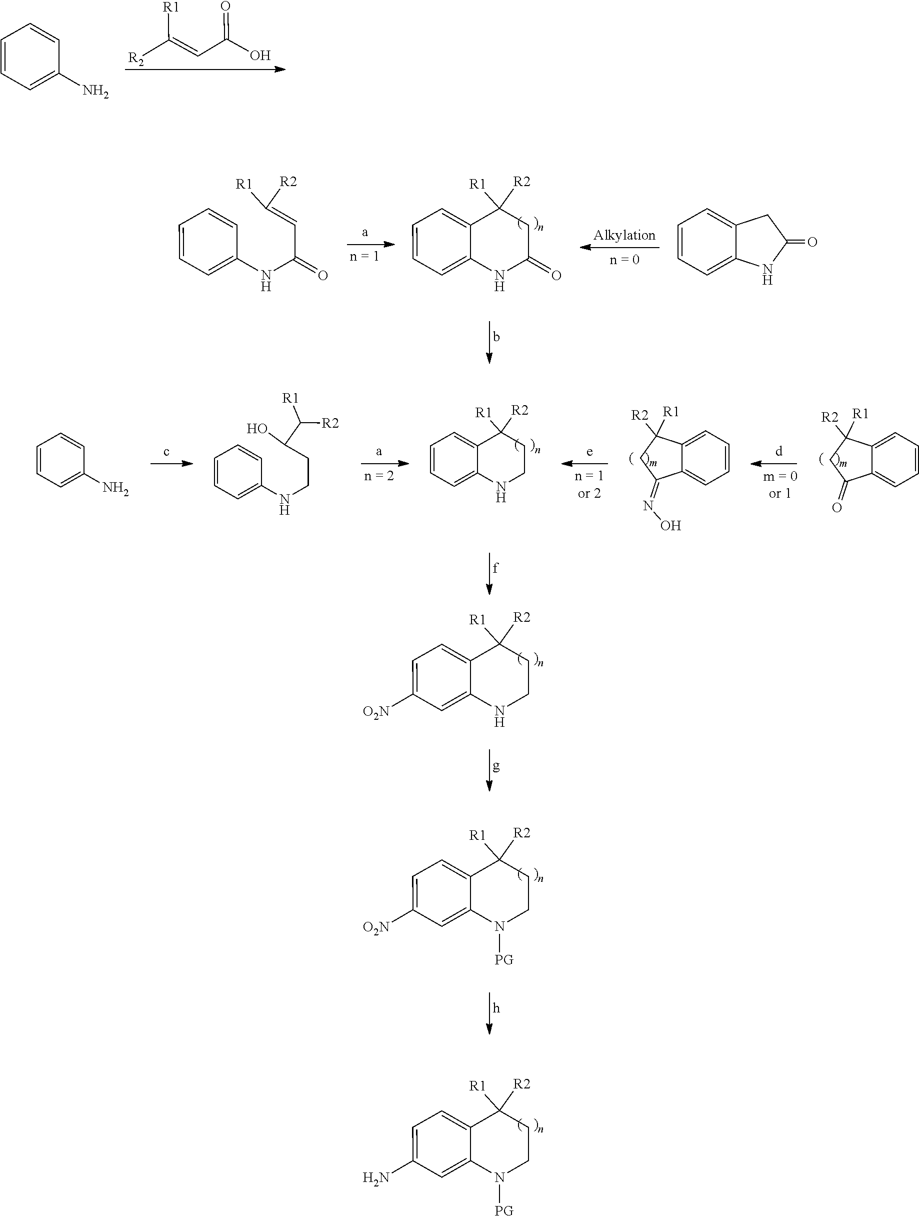

a) AlCl₃; b) [H]; c) i) R1R2CHCOCH₂CH₂Cl; ii) NaBH₄; d) NH₂OH; e) DIBAL-H; f) nitration; g) protection; h) [H]
PG=protecting group

Synthesis of Amine Precursors P-V-B-3:

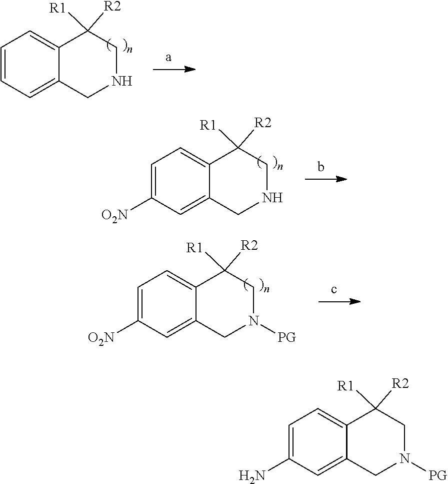

### a) Nitration; b) Protection; c) [H]

PG=protecting group

Synthesis of Amine Precursors P-V-B-5:

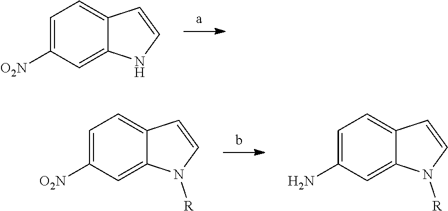

a) when X=Cl, Br, I: RX, K₂CO₃, DMF or CH₃CN; when X=OH: RX, TFFH, DIEA, THF b) H₂, Pd—C, EtOH or SnCl₂.2H₂O, EtOH or SnCl₂.2H₂O, DIEA, EtOH.

Synthesis of Amine Precursors P-V-B-5:

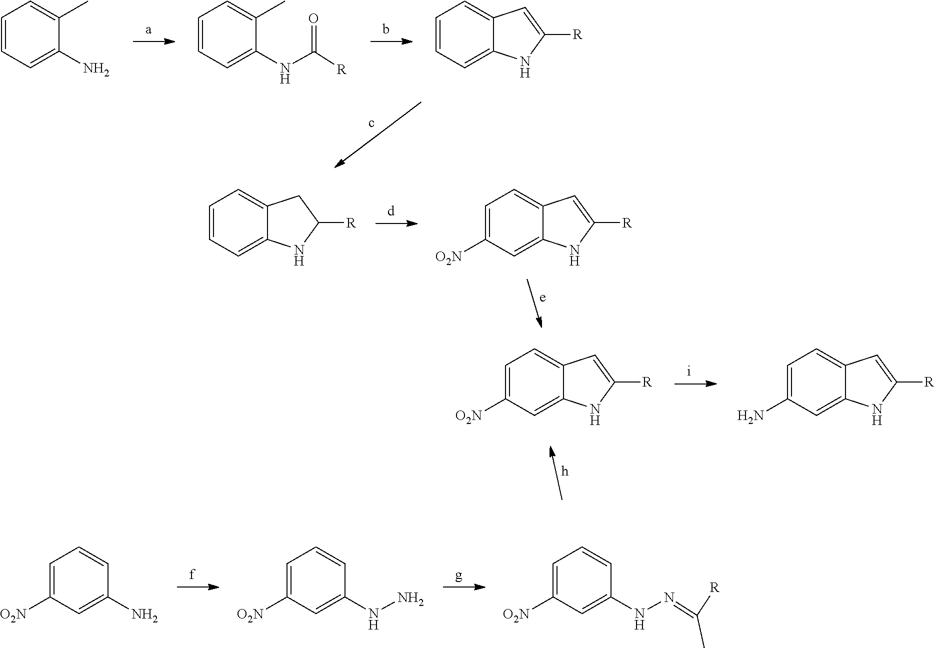

a) RCOCl, Et₃N, CH₂Cl₂; b) n-BuLi, THF; c) NaBH₄, AcOH; d) KNO₃, H₂SO₄; e) DDQ, 1,4-dioxane; f) NaNO₂, HCl, SnCl₂.2H₂O, H₂O; g) MeCOR, EtOH; h) PPA; i) LiAlH₄, THF or H₂, Raney Ni, EtOH or MeOH

Synthesis of Amine Precursors V-B-5:

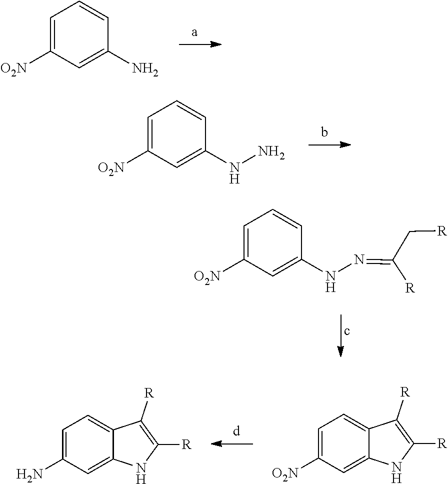

a) NaNO₂, HCl, SnCl₂.2H₂O, H₂O; b) RCH₂COR, AcOH, EtOH; c) H₃PO₄, toluene; d) H₂, Pd—C, EtOH

Synthesis of Amine Precursors P-V-B-5:

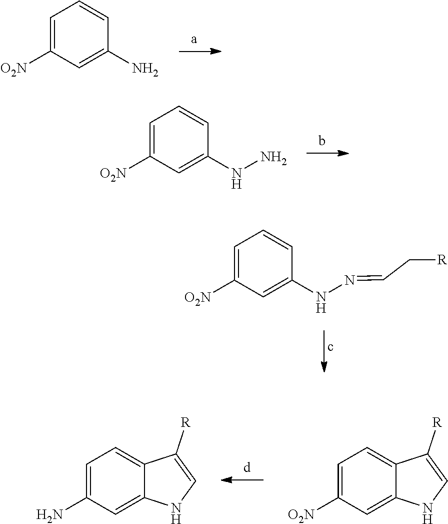

a) NaNO₂, HCl, SnCl₂.2H₂O, H₂O; b) RCH₂COH, AcOH, EtOH; c) H₃PO₄, toluene; d) H₂, Pd—C, EtOH

Synthesis of Amine Precursors P-V-B-5:

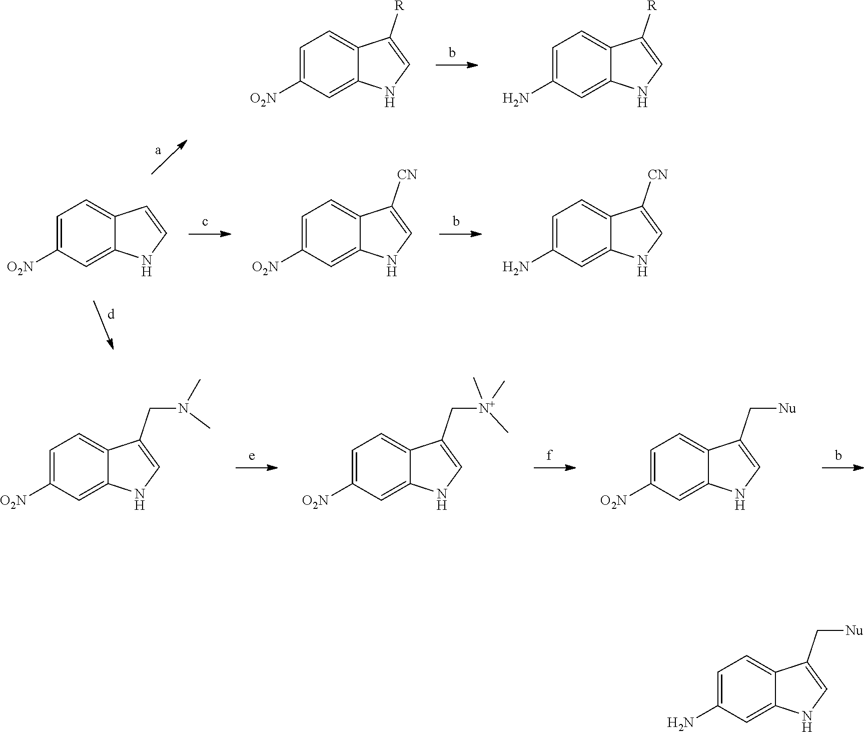

a) RX (X═Br, I), zinc triflate, TBAI, DIEA, toluene; b) H₂, Raney Ni, EtOH or H₂, Pd—C, EtOH or SnCl₂.2H₂O, EtOH; c) ClSO₂NCO, DMF, CH₃CN; d) Me₂NH, H₂CO, AcOH; e) MeI, DMF, THF, H₂O; f) MNu (M=Na, K, Li; Nu=nucleophile)

Synthesis of Amine Precursors P-V-B-5:

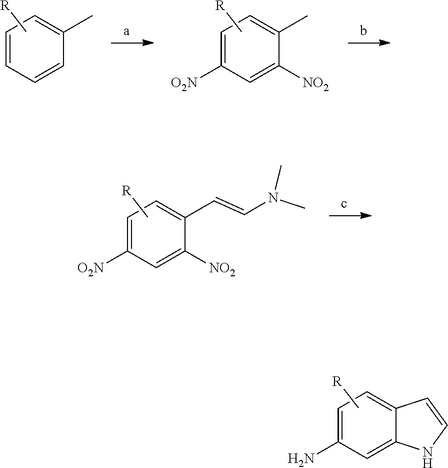

### a) HNO₃, H₂SO₄; b) Me₂NCH(OMe)₂, DMF; c) H₂, Raney Ni, EtOH

Synthesis of Amine Precursors P-V-B-5:

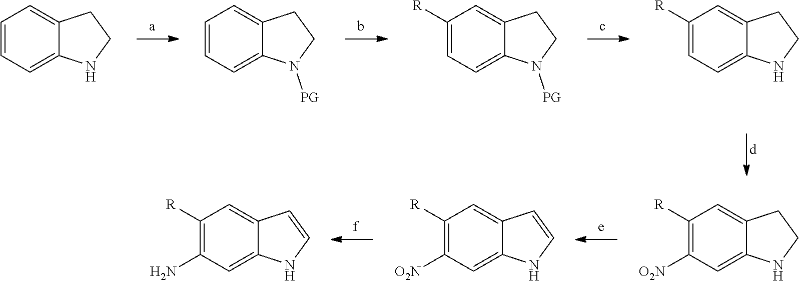

a) When PG=SO₂Ph: PhSO₂Cl, Et₃N, DMAP, CH₂Cl₂; When PG=Ac: AcCl, NaHCO₃, CH₂Cl₂; b) When R═RCO: (RCO)₂O, AlCl₃, CH₂Cl₂; When R=Br: Br₂, AcOH; c) HBr or HCl; d) KNO₃, H₂SO₄; e) MnO₂, CH₂Cl₂ or DDQ, 1,4-dioxane; f) H₂, Raney Ni, EtOH.

Synthesis of Amine Precursors P-V-B-5:

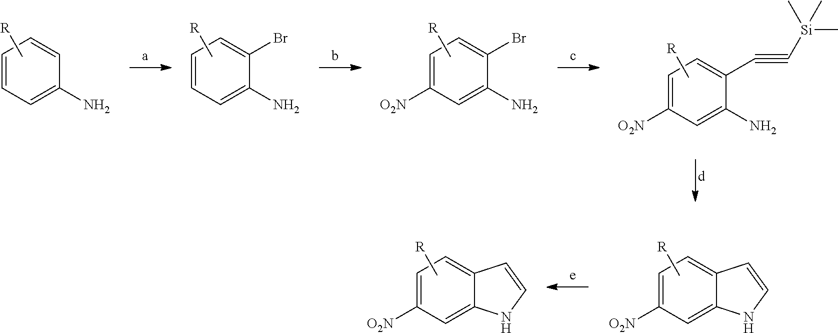

a) NBS, DMF; b) KNO₃, H₂SO₄; c) HC═CSiMe₃, Pd(PPh₃)₂Cl₂, CuI, Et₃N, Toluene, H₂O; d) CuI, DMF; e) H₂, Raney Ni, MeOH

Synthesis of Amine Precursors P-V-A-3 and P-V-A-6:

Ar=Aryl or heteroaryl

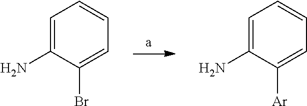

a) ArB(OH)₂, Pd(PPh₃)₄, K₂CO₃, H₂O, THF or ArB(OH)₂, Pd₂(dba)₃, P(tBu)₃, KF, THF

Synthesis of Amine Precursors P-V-A-4:

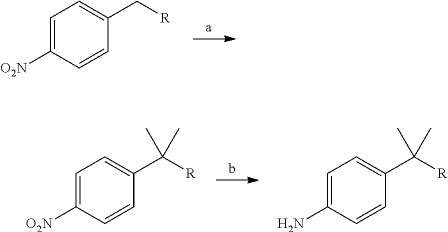

### R═CN, CO₂Et; a) MeI, NaOtBu, DMF; b) HCO₂K, Pd—C, EtOH or HCO₂NH₄, Pd—C, EtOH

Synthesis of Amine Precursors P-V-A-4:

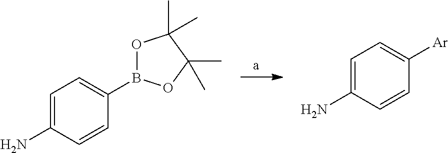

### a) ArBr, Pd(OAc)₂, PS—PPh₃, K₂CO₃, DMF

Synthesis of Amine Precursors P-V-B-4:

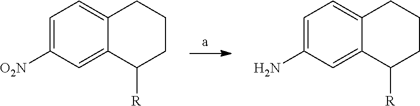

### a) H₂, Pd—C, MeOH

Synthesis of Amine Precursors P-V-B-4:

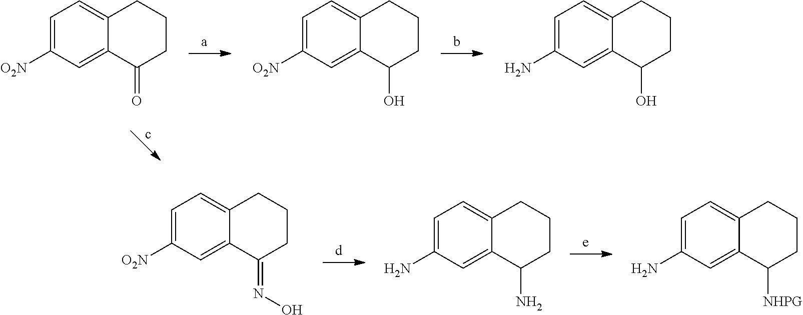

### a) NaBH₄, MeOH; b) H₂, Pd—C, MeOH; c) NH₂OH, Pyridine; d) H₂, Pd—C, MeOH; e) Boc₂O, Et₃N, MeOH

Synthesis of Compounds of Formula A:

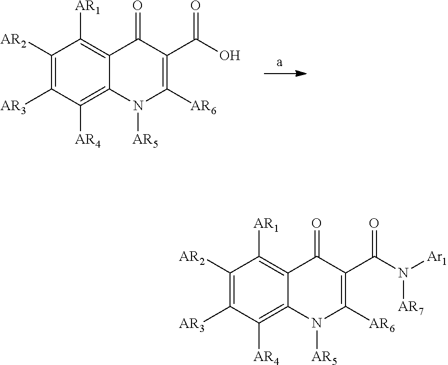

a) Ar₁R⁷NH, coupling reagent, base, solvent. Examples of conditions used:

HATU, DIEA; BOP, DIEA, DMF; HBTU, Et₃N, CH₂Cl₂; PFPTFA, pyridine.

Synthesis of Compounds of Formula AI′:

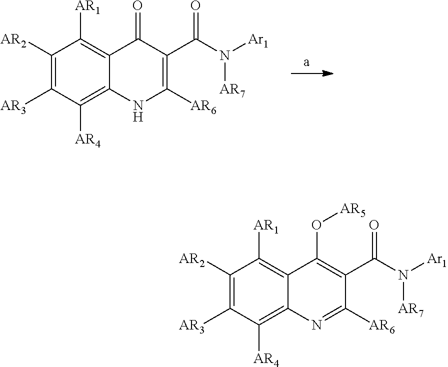

R⁵=aliphatic: a) R⁵X (X=Br, I), Cs₂CO₃, DMF

Synthesis of Compounds of formula AVB-5:

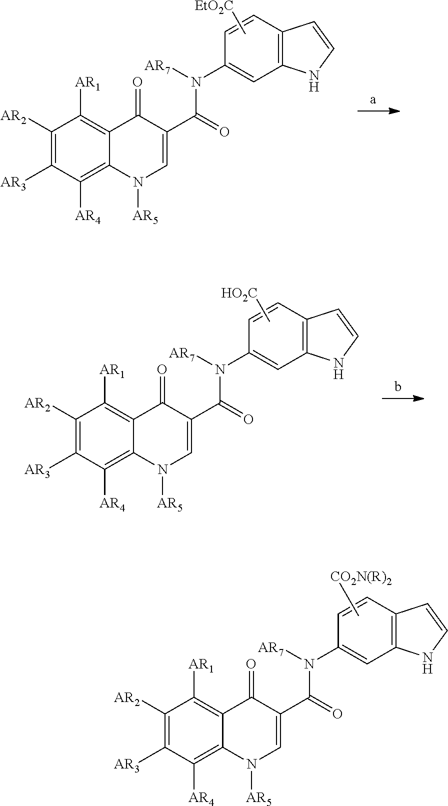

### a) NaOH, THF; b) HNR₂, HATU, DIEA, DMF

Synthesis of Compounds of formula AVB-5:

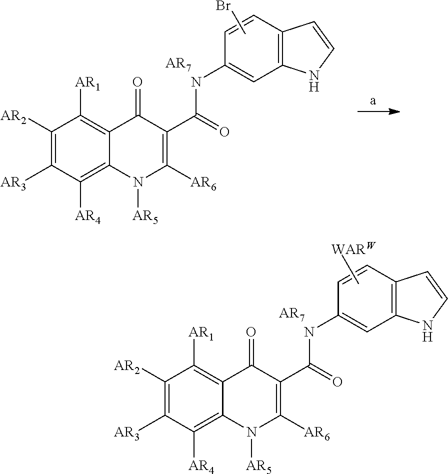

WAR^{w}=aryl or heteroaryl: a) ArB(OH)₂, (dppt)PdCl₂, K₂CO₃, DMF

Synthesis of Compounds of Formula AVA-2 & AVA-5:

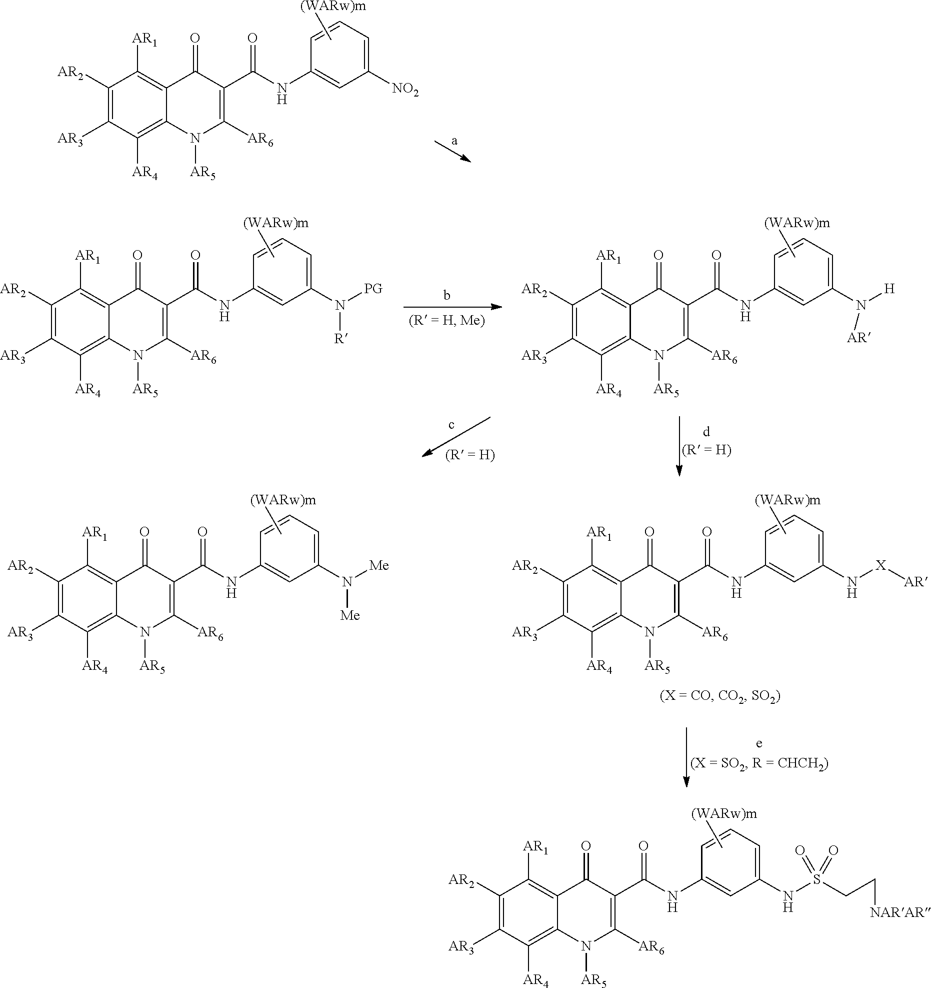

a) SnCl₂.2H₂O, EtOH; b) PG=BOC: TFA, CH₂Cl₂; c) CH₂O, NaBH₃CN, CH₂Cl₂, MeOH; d) ARXCl, DIEA, THF or ARXCl, NMM, 1,4-dioxane or ARXCl, CH₂Cl₂, DMF; e) AR′AR″NH, LiClO₄, CH₂Cl₂, iPrOH

Synthesis of Compounds of Formula AVB-2:

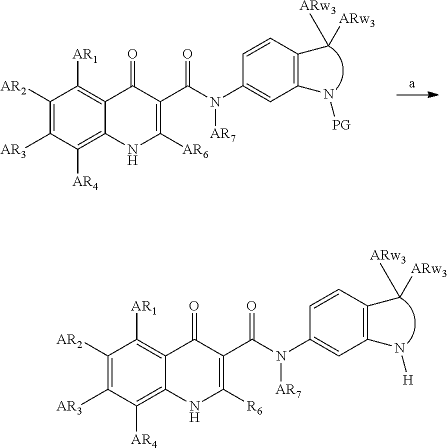

### a) When PG=BOC: TFA, CH₂Cl₂; When PG=Ac: NaOH or HCl, EtOH or THF

Synthesis of Compounds of Formula AVA-2:

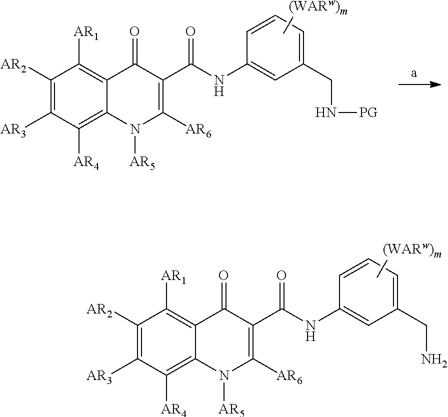

### a) When PG=BOC: TFA, CH₂Cl₂

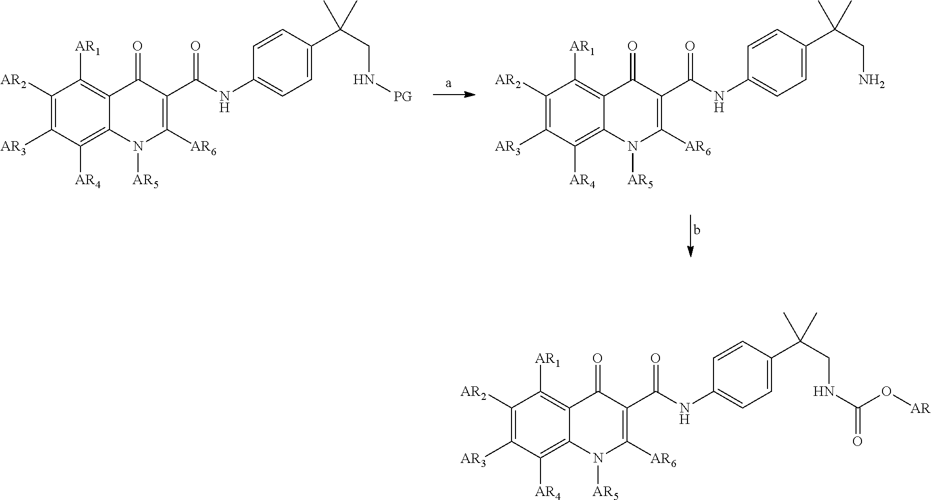

### a) When PG=BOC: TFA, CH₂Cl₂; b) ROCOCl, Et₃N, DMF

Synthesis of Compounds of Formula AVA-4:

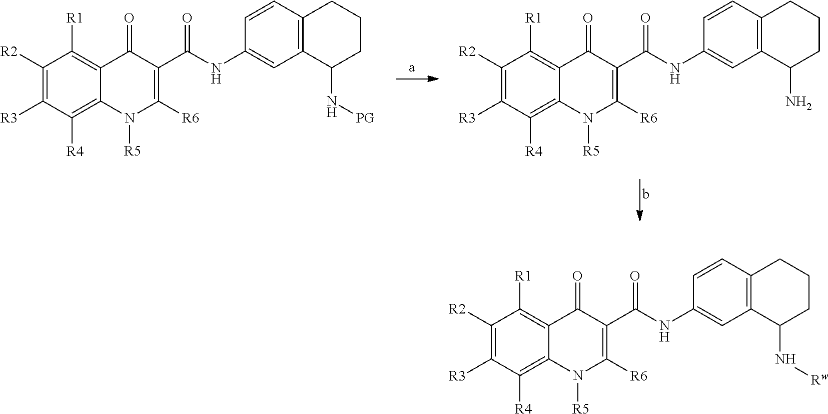

### a) When PG=BOC: TFA, CH₂Cl₂; b) When R^{w}=CO₂R: ROCOCl, DIEA, MeOH

In the schemes herein, the radical R, R′ etc. employed therein is a substituent, e.g., AR^{W}, as defined hereinabove. One of skill in the art will readily appreciate that synthetic routes suitable for various substituents of the present invention are such that the reaction conditions and steps employed do not modify the intended substituents.

### Example 1

### General Scheme to Prepare Acid Moities

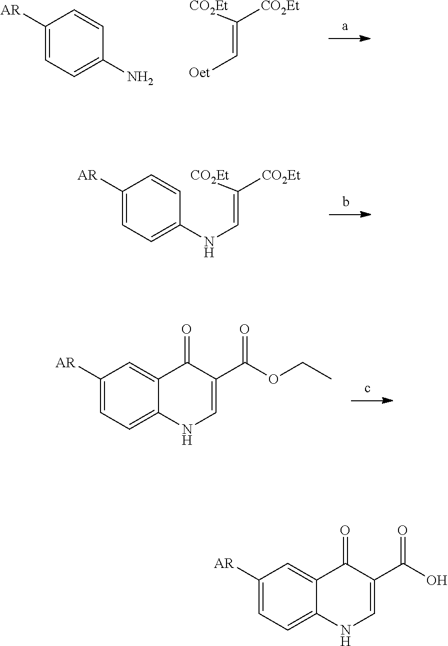

a) 140-150° C.; b) PPA, POCl₃, 70° C. or diphenyl ether, 220° C.; c) i) 2N NaOH ii) 2N HCl

### Specific Example

### 2-Phenylaminomethylene-Malonic Acid Diethyl Ester

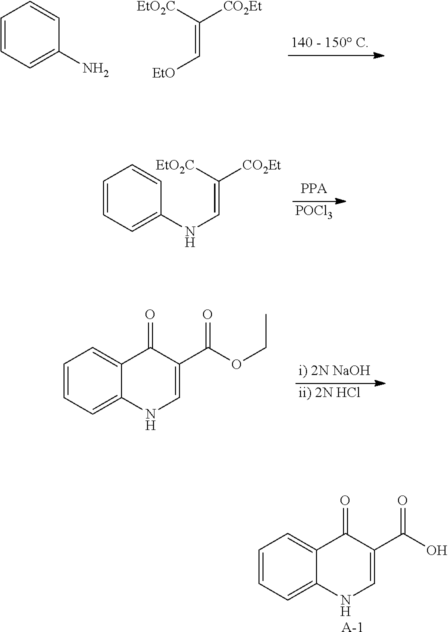

A mixture of aniline (25.6 g, 0.28 mol) and diethyl 2-(ethoxymethylene)malonate (62.4 g, 0.29 mol) was heated at 140-150° C. for 2 h. The mixture was cooled to room temperature and dried under reduced pressure to afford 2-phenylaminomethylene-malonic acid diethyl ester as a solid, which was used in the next step without further purification. ¹H NMR (d-DMSO) δ 11.00 (d, 1H), 8.54 (d, J=13.6 Hz, 1H), 7.36-7.39 (m, 2H), 7.13-7.17 (m, 3H), 4.17-4.33 (m, 4H), 1.18-1.40 (m, 6H).

### 4-Hydroxyquinoline-3-Carboxylic Acid Ethyl Ester

A 1 L three-necked flask fitted with a mechanical stirrer was charged with 2-phenylaminomethylene-malonic acid diethyl ester (26.3 g, 0.1 mol), polyphosphoric acid (270 g) and phosphoryl chloride (750 g). The mixture was heated to about 70° C. and stirred for 4 h. The mixture was cooled to room temperature, and filtered. The residue was treated with aqueous Na₂CO₃ solution, filtered, washed with water and dried. 4-Hydroxyquinoline-3-carboxylic acid ethyl ester was obtained as a pale brown solid (15.2 g, 70%). The crude product was used in next step without further purification.

### A-1; 4-Oxo-1,4-dihydroquinoline-3-carboxylic acid

4-Hydroxyquinoline-3-carboxylic acid ethyl ester (15 g, 69 mmol) was suspended in sodium hydroxide solution (2N, 150 mL) and stirred for 2 h under reflux. After cooling, the mixture was filtered, and the filtrate was acidified to pH 4 with 2N HCl. The resulting precipitate was collected via filtration, washed with water and dried under vacuum to give 4-oxo-1,4-dihydroquinoline-3-carboxylic acid (A-1) as a pale white solid (10.5 g, 92%). ¹H NMR (d-DMSO) δ 15.34 (s, 1H), 13.42 (s, 1H), 8.89 (s, 1H), 8.28 (d, J=8.0 Hz, 1H), 7.88 (m, 1H), 7.81 (d, J=8.4 Hz, 1H), 7.60 (m, 1H).

### Specific Example

### A-2; 6-Fluoro-4-hydroxy-quinoline-3-carboxylic acid

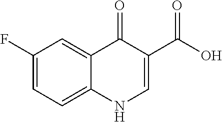 [SMILES: O=C(O)c1c[nH]c2ccc(F)cc2c1=O]

6-Fluoro-4-hydroxy-quinoline-3-carboxylic acid (A-2) was synthesized following the general scheme above starting from 4-fluoro-phenylamine. Overall yield (53%). ¹H NMR (DMSO-d₆) δ 15.2 (br s, 1H), 8.89 (s, 1H), 7.93-7.85 (m, 2H), 7.80-7.74 (m, 1H); ESI-MS 207.9 m/z (MH⁺).

### Example 2

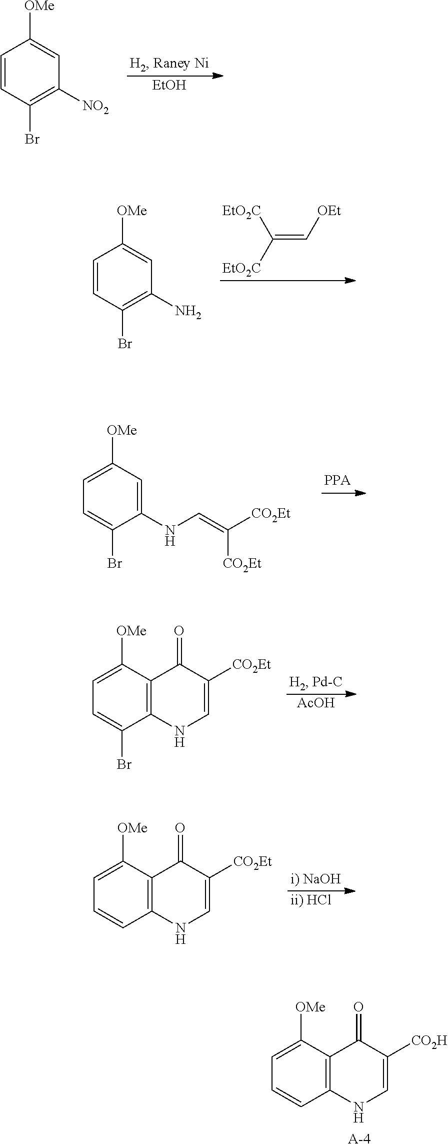

### 2-Bromo-5-methoxy-phenylamine

A mixture of 1-bromo-4-methoxy-2-nitro-benzene (10 g, 43 mmol) and Raney Ni (5 g) in ethanol (100 mL) was stirred under H₂ (1 atm) for 4 h at room temperature. Raney Ni was filtered off and the filtrate was concentrated under reduced pressure. The resulting solid was purified by column chromatography to give 2-bromo-5-methoxy-phenylamine (7.5 g, 86%).

### 2-[(2-Bromo-5-methoxy-phenylamino)-methylene]-malonic acid diethyl ester

A mixture of 2-bromo-5-methoxy-phenylamine (540 mg, 2.64 mmol) and diethyl 2-(ethoxymethylene)malonate (600 mg, 2.7 mmol) was stirred at 100° C. for 2 h. After cooling, the reaction mixture was recrystallized from methanol (10 mL) to give 2-[(2-bromo-5-methoxy-phenylamino)-methylene]-malonic acid diethyl ester as a yellow solid (0.8 g, 81%).

### 8-Bromo-5-methoxy-4-oxo-1,4-dihydro-quinoline-3-carboxylic acid ethyl ester

2-[(2-Bromo-5-methoxy-phenylamino)-methylene]-malonic acid diethyl ester (9 g, 24.2 mmol) was slowly added to polyphosphoric acid (30 g) at 120° C. The mixture was stirred at this temperature for additional 30 min and then cooled to room temperature. Absolute ethanol (30 mL) was added and the resulting mixture was refluxed for 30 min. The mixture was basified with aqueous sodium bicarbonate at 25° C. and extracted with EtOAc (4×100 mL). The organic layers were combined, dried and the solvent evaporated to give 8-bromo-5-methoxy-4-oxo-1,4-dihydro-quinoline-3-carboxylic acid ethyl ester (2.3 g, 30%).

### 5-Methoxy-4-oxo-1,4-dihydro-quinoline-3-carboxylic acid ethyl ester

A mixture of 8-bromo-5-methoxy-4-oxo-1,4-dihydro-quinoline-3-carboxylic acid ethyl ester (2.3 g, 7.1 mmol), sodium acetate (580 mg, 7.1 mmol) and 10% Pd/C (100 mg) in glacial acetic acid (50 ml) was stirred under H₂ (2.5 atm) overnight. The catalyst was removed via filtration, and the reaction mixture was concentrated under reduced pressure. The resulting oil was dissolved in CH₂Cl₂ (100 mL) and washed with aqueous sodium bicarbonate solution and water. The organic layer was dried, filtered and concentrated. The crude product was purified by column chromatography to afford 5-methoxy-4-oxo-1,4-dihydro-quinoline-3-carboxylic acid ethyl ester as a yellow solid (1 g, 57%).

### A-4; 5-Methoxy-4-oxo-1,4-dihydro-quinoline-3-carboxylic acid

A mixture of 5-methoxy-4-oxo-1,4-dihydro-quinoline-3-carboxylic acid ethyl ester (1 g, 7.1 mmol) in 10% NaOH solution (50 mL) was heated to reflux overnight and then cooled to room temperature. The mixture was extracted with ether. The aqueous phase was separated and acidified with conc. HCl solution to pH 1-2. The resulting precipitate was collected by filtration to give 5-methoxy-4-oxo-1,4-dihydro-quinoline-3-carboxylic acid (A-4) (530 mg, 52%). ¹H NMR (DMSO) δ: 15.9 (s, 1H), 13.2 (br, 1H), 8.71 (s, 1H), 7.71 (t, J=8.1 Hz, 1H), 7.18 (d, J=8.4 Hz, 1H), 6.82 (d, J=8.4 Hz, 1H), 3.86 (s, 3H); ESI-MS 219.9 m/z (MH⁺).

### Example 3

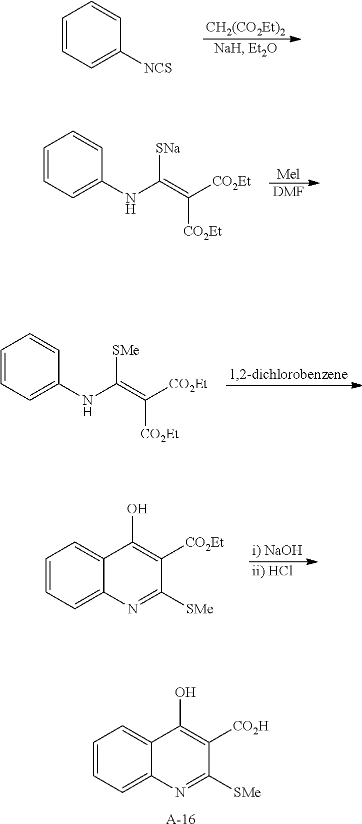

### Sodium 2-(mercapto-phenylamino-methylene)-malonic acid diethyl ester

To a suspension of NaH (60% in mineral oil, 6 g, 0.15 mol) in Et₂O at room temperature was added dropwise, over a 30 minutes period, ethyl malonate (24 g, 0.15 mol). Phenyl isothiocyanate (20.3 g, 0.15 mol) was then added dropwise with stirring over 30 min. The mixture was refluxed for 1 h and then stirred overnight at room temperature. The solid was separated, washed with anhydrous ether (200 mL), and dried under vacuum to yield sodium 2-(mercapto-phenylamino-methylene)-malonic acid diethyl ester as a pale yellow powder (46 g, 97%).

### 2-(Methylsulfanyl-phenylamino-methylene)-malonic acid diethyl ester

Over a 30 min period, methyl iodide (17.7 g, 125 mmol) was added dropwise to a solution of sodium 2-(mercapto-phenylamino-methylene)-malonic acid diethyl ester (33 g, 104 mmol) in DMF (100 mL) cooled in an ice bath. The mixture was stirred at room temperature for 1 h, and then poured into ice water (300 mL). The resulting solid was collected via filtration, washed with water and dried to give 2-(methylsulfanyl-phenylamino-methylene)-malonic acid diethyl ester as a pale yellow solid (27 g, 84%).

### 4-Hydroxy-2-methylsulfanyl-quinoline-3-carboxylic acid ethyl ester

A mixture of 2-(methylsulfanyl-phenylamino-methylene)-malonic acid diethyl ester (27 g, 87 mmol) in 1,2-dichlorobenzene (100 mL) was heated to reflux for 1.5 h. The solvent was removed under reduced pressure and the oily residue was triturated with hexane to afford a pale yellow solid that was purified by preparative HPLC to yield 4-hydroxy-2-methylsulfanyl-quinoline-3-carboxylic acid ethyl ester (8 g, 35%).

### A-16; 2-Methylsulfanyl-4-oxo-1,4-dihydro-quinoline-3-carboxylic acid

4-Hydroxy-2-methylsulfanyl-quinoline-3-carboxylic acid ethyl ester (8 g, 30 mmol) was heated under reflux in NaOH solution (10%, 100 mL) for 1.5 h. After cooling, the mixture was acidified with concentrated HCl to pH 4. The resulting solid was collected via filtration, washed with water (100 mL) and MeOH (100 mL) to give 2-methylsulfanyl-4-oxo-1,4-dihydro-quinoline-3-carboxylic acid (A-16) as a white solid (6 g, 85%). ¹H NMR (CDCl₃) δ 16.4 (br s, 1H), 11.1 (br s, 1H), 8.19 (d, J=8 Hz, 1H), 8.05 (d, J=8 Hz, 1H), 7.84 (t, J=8, 8 Hz, 1H), 7.52 (t, J=8 Hz, 1H), 2.74 (s, 3H); ESI-MS 235.9 m/z (MH⁺).

### Example 4

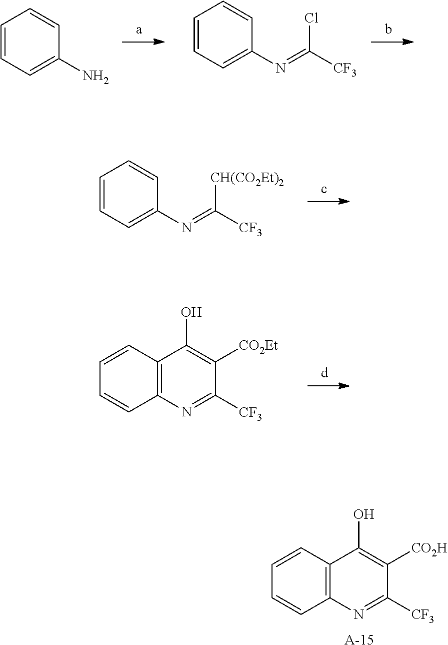

a) PPh₃, Et₃N, CCl₄, CF₃CO₂H; b) diethyl malonate; c) 200° C.; d) 10% NaOH

### 2,2,2-Trifluoro-N-phenyl-acetimidoyl chloride

A mixture of Ph₃P (138.0 g, 526 mmol), Et₃N (21.3 g, 211 mmol), CCl₄ (170 mL) and TFA (20 g, 175 mmol) was stirred for 10 min in an ice-bath. Aniline (19.6 g, 211 mmol) was dissolved in CCl₄ (20 mL) was added. The mixture was stirred at reflux for 3 h. The solvent was removed under vacuum and hexane was added. The precipitates (Ph₃PO and Ph₃P) were filtered off and washed with hexane. The filtrate was distilled under reduced pressure to yield 2,2,2-trifluoro-N-phenyl-acetimidoyl chloride (19 g), which was used in the next step without further purification.

### 2-(2,2,2-Trifluoro-1-phenylimino-ethyl)-malonic acid diethyl ester

To a suspension of NaH (3.47 g, 145 mmol, 60% in mineral oil) in THF (200 mL) was added diethyl malonate (18.5 g, 116 mmol) at 0° C. The mixture was stirred for 30 min at this temperature and 2,2,2-trifluoro-N-phenyl-acetimidoyl chloride (19 g, 92 mmol) was added at 0° C. The reaction mixture was allowed to warm to room temperature and stirred overnight. The mixture was diluted with CH₂Cl₂, washed with saturated sodium bicarbonate solution and brine. The combined organic layers were dried over Na₂SO₄, filtered and concentrated to provide 2-(2,2,2-trifluoro-1-phenylimino-ethyl)-malonic acid diethyl ester, which was used directly in the next step without further purification.

### 4-Hydroxy-2-trifluoromethyl-quinoline-3-carboxylic acid ethyl ester

2-(2,2,2-Trifluoro-1-phenylimino-ethyl)-malonic acid diethyl ester was heated at 210° C. for 1 h with continuous stirring. The mixture was purified by column chromatography (petroleum ether) to yield 4-hydroxy-2-trifluoromethyl-quinoline-3-carboxylic acid ethyl ester (12 g, 24% over 3 steps).

### A-15; 4-Hydroxy-2-trifluoromethyl-quinoline-3-carboxylic acid

A suspension of 4-hydroxy-2-trifluoromethyl-quinoline-3-carboxylic acid ethyl ester (5 g, 17.5 mmol) in 10% aqueous NaOH solution was heated at reflux for 2 h. After cooling, dichloromethane was added and the aqueous phase was separated and acidified with concentrated HCl to pH 4. The resulting precipitate was collected via filtration, washed with water and Et₂O to provide 4-hydroxy-2-trifluoromethyl-quinoline-3-carboxylic acid (A-15) (3.6 g, 80%). ¹H NMR (DMSO-d₆) δ 8.18-8.21 (d, J=7.8 Hz, 1H), 7.92-7.94 (d, J=8.4 Hz, 1H), 7.79-7.83 (t, J=14.4 Hz, 1H), 7.50-7.53 (t, J=15 Hz, 1H); ESI-MS 257.0 m/z (MH⁺).

### Example 5

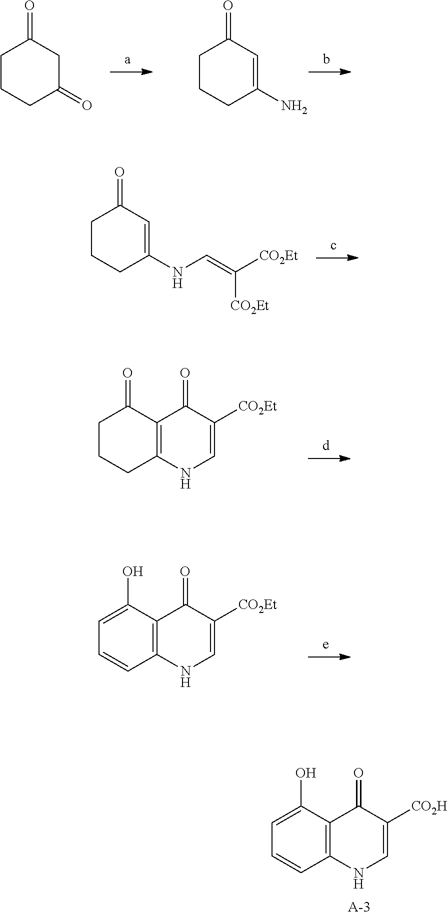

a) CH₃C(O)ONH₄, toluene; b) EtOCHC(CO₂Et)₂, 130° C.; c) Ph₂O; d) I₂, EtOH; e) NaOH

### 3-Amino-cyclohex-2-enone

A mixture of cyclohexane-1,3-dione (56.1 g, 0.5 mol) and AcONH₄ (38.5 g, 0.5 mol) in toluene was heated at reflux for 5 h with a Dean-stark apparatus. The resulting oily layer was separated and concentrated under reduced pressure to give 3-amino-cyclohex-2-enone (49.9 g, 90%), which was used directly in the next step without further purification.

### 2-[(3-Oxo-cyclohex-1-enylamino)-methylene]malonic acid diethyl ester

A mixture of 3-amino-cyclohex-2-enone (3.3 g, 29.7 mmol) and diethyl 2-(ethoxymethylene)malonate (6.7 g, 31.2 mmol) was stirred at 130° C. for 4 h. The reaction mixture was concentrated under reduced pressure and the resulting oil was purified by column chromatography (silica gel, ethyl acetate) to give 2-[(3-oxo-cyclohex-1-enylamino)-methylene]-malonic acid diethyl ester (7.5 g, 90%).

### 4,5-Dioxo-1,4,5,6,7,8-hexahydro-quinoline-3-carboxylic acid ethyl ester

A mixture of 2-[(3-oxo-cyclohex-1-enylamino)-methylene]-malonic acid diethyl ester (2.8 g, 1 mmol) and diphenylether (20 mL) was refluxed for 15 min. After cooling, n-hexane (80 mL) was added. The resulting solid was isolated via filtration and recrystallized from methanol to give 4,5-dioxo-1,4,5,6,7,8-hexahydro-quinoline-3-carboxylic acid ethyl ester (1.7 g 72%).

### 5-Hydroxy-4-oxo-1,4-dihydro-quinoline-3-carboxylic acid ethyl ester

To a solution of 4,5-dioxo-1,4,5,6,7,8-hexahydro-quinoline-3-carboxylic acid ethyl ester (1.6 g, 6.8 mmol) in ethanol (100 mL) was added iodine (4.8 g, 19 mmol). The mixture was refluxed for 19 h and then concentrated under reduced pressure. The resulting solid was washed with ethyl acetate, water and acetone, and then recrystallized from DMF to give 5-hydroxy-4-oxo-1,4-dihydro-quinoline-3-carboxylic acid ethyl ester (700 mg, 43%).

### A-3; 5-Hydroxy-4-oxo-1,4-dihydro-quinoline-3-carboxylic acid

A mixture of 5-hydroxy-4-oxo-1,4-dihydro-quinoline-3-carboxylic acid ethyl ester (700 mg, 3 mmol) in 10% NaOH (20 ml) was heated at reflux overnight. After cooling, the mixture was extracted with ether. The aqueous phase was separated and acidified with conc. HCl to pH 1-2. The resulting precipitate was collected via filtration to give 5-hydroxy-4-oxo-1,4-dihydro-quinoline-3-carboxylic acid (A-3) (540 mg, 87%). ¹H NMR (DMSO-d₆) δ 13.7 (br, 1H), 13.5 (br, 1H), 12.6 (s, 1H), 8.82 (s, 1H), 7.68 (t, J=8.1 Hz, 1H), 7.18 (d, J=8.4 Hz, 1H), 6.82 (d, J=8.4 Hz, 1H); ESI-MS 205.9 m/z (MH⁺).

### Example 6

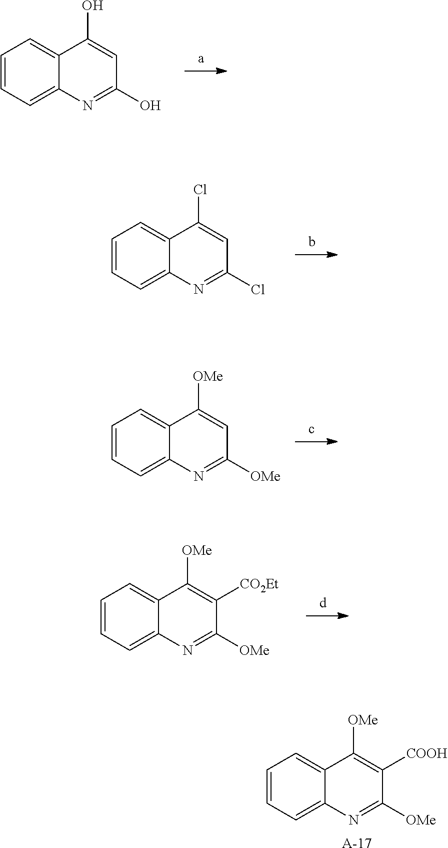

a) POCl₃; b) MeONa; c) n-BuLi, ClCO₂Et; d) NaOH

### 2,4-Dichloroquinoline

A suspension of quinoline-2,4-diol (15 g, 92.6 mmol) in POCl₃ was heated at reflux for 2 h. After cooling, the solvent was removed under reduced pressure to yield 2,4-dichloroquinoline, which was used without further purification.

### 2,4-Dimethoxyquinoline

To a suspension of 2,4-dichloroquinoline in MeOH (100 mL) was added sodium methoxide (50 g). The mixture was heated at reflux for 2 days. After cooling, the mixture was filtered. The filtrate was concentrated under reduced pressure to yield a residue that was dissolved in water and extracted with CH₂Cl₂. The combined organic layers were dried over Na₂SO₄ and concentrated to give 2,4-dimethoxyquinoline as a white solid (13 g, 74% over 2 steps).

### Ethyl 2,4-dimethoxyquinoline-3-carboxylate

To a solution of 2,4-dimethoxyquinoline (11.5 g, 60.8 mmol) in anhydrous THF was added dropwise n-BuLi (2.5 Min hexane, 48.6 mL, 122 mmol) at 0° C. After stirring for 1.5 h at 0° C., the mixture was added to a solution of ethyl chloroformate in anhydrous THF and stirred at 0° C. for additional 30 min and then at room temperature overnight. The reaction mixture was poured into water and extracted with CH₂Cl₂. The organic layer was dried over Na₂SO₄ and concentrated under vacuum. The resulting residue was purified by column chromatography (petroleum ether/EtOAc=50/1) to give ethyl 2,4-dimethoxyquinoline-3-carboxylate (9.6 g, 60%).

### A-17; 2,4-Dimethoxyquinoline-3-carboxylic acid

Ethyl 2,4-dimethoxyquinoline-3-carboxylate (1.5 g, 5.7 mmol) was heated at reflux in NaOH solution (10%, 100 mL) for 1 h. After cooling, the mixture was acidified with concentrated HCl to pH 4. The resulting precipitate was collected via filtration and washed with water and ether to give 2,4-dimethoxyquinoline-3-carboxylic acid (A-17) as a white solid (670 mg, 50%). ¹H NMR (CDCl₃) δ 8.01-8.04 (d, J=12 Hz, 1H), 7.66-7.76 (m, 2H), 7.42-7.47 (t, J=22 Hz, 2H), 4.09 (s, 3H). 3.97 (s, 3H); ESI-MS 234.1 m/z (MH⁺).

TABLE II.A-2
Commercially available acids

| Acid | Name |
|---|---|
| A-5 | 6,8-Difluoro-4-oxo-1,4-dihydro-quinoline-3-carboxylic acid |
| A-6 | 6-[(4-Fluoro-phenyl)-methyl-sulfamoyl]-4-oxo-1,4-dihydro- |
| | quinoline-3-carboxylic acid |
| A-7 | 6-(4-Methyl-piperidine-1-sulfonyl)-4-oxo-1,4-dihydro-quinoline-3- |
| | carboxylic acid |
| A-8 | 4-Oxo-6-(pyrrolidine-1-sulfonyl)-1,4-dihydro-quinoline-3- |
| | carboxylic acid |
| A-10 | 6-Ethyl-4-oxo-1,4-dihydro-quinoline-3-carboxylic acid |
| A-11 | 6-Ethoxy-4-oxo-1,4-dihydro-quinoline-3-carboxylic acid |
| A-12 | 4-Oxo-7-trifluoromethyl-1,4-dihydro-quinoline-3-carboxylic acid |
| A-13 | 7-Chloro-4-oxo-1,4-dihydro-quinoline-3-carboxylic acid |
| A-14 | 4-Oxo-5,7-bis-trifluoromethyl-1,4-dihydro-quinoline-3-carboxylic |
| | acid |
| A-20 | 1-Methyl-4-oxo-1,4-dihydro-quinoline-3-carboxylic acid |
| A-21 | 1-Isopropyl-4-oxo-1,4-dihydro-quinoline-3-carboxylic acid |
| A-22 | 1,6-Dimethyl-4-oxo-1,4-dihydro-quinoline-3-carboxylic acid |
| A-23 | 1-Ethyl-6-methoxy-4-oxo-1,4-dihydro-quinoline-3-carboxylic acid |
| A-24 | 6-Chloro-4-oxo-1,4-dihydro-quinoline-3-carboxylic acid |

Amine Moieties

N-1 Substituted 6-aminoindoles

### Example 1

### General Scheme

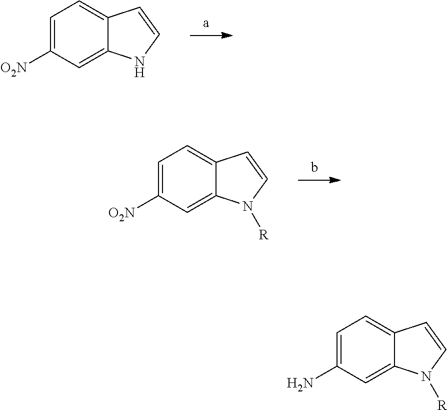

### a) RX (X=Cl, Br, I), K₂CO₃, DMF or CH₃CN; b) H₂, Pd—C, EtOH or SnCl₂.2H₂O, EtOH.

### Specific Example

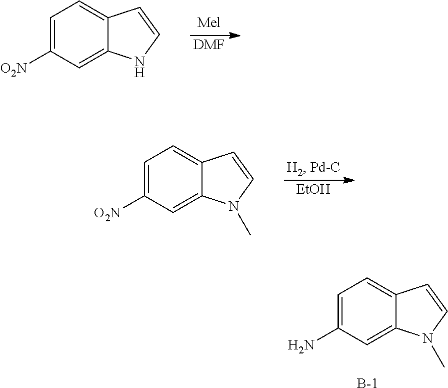

### 1-Methyl-6-nitro-1H-indole

To a solution of 6-nitroindole (4.05 g 25 mmol) in DMF (50 mL) was added K₂CO₃ (8.63 g, 62.5 mmol) and MeI (5.33 g, 37.5 mmol). After stirring at room temperature overnight, the mixture was poured into water and extracted with ethyl acetate. The combined organic layers were dried over Na₂SO₄ and concentrated under vacuum to give the product 1-methyl-6-nitro-1H-indole (4.3 g, 98%).

### B-1; 1-Methyl-1H-indol-6-ylamine

A suspension of 1-methyl-6-nitro-1H-indole (4.3 g, 24.4 mmol) and 10% Pd—C (0.43 g) in EtOH (50 mL) was stirred under H₂ (1 atm) at room temperature overnight. After filtration, the filtrate was concentrated and acidified with HCl-MeOH (4 mol/L) to give 1-methyl-1H-indol-6-ylamine hydrochloride salt (B-1) (1.74 g, 49%) as a grey powder. ¹H NMR (DMSO-d₆): δ 9.10 (s, 2H), 7.49 (d, J=8.4 Hz, 1H), 7.28 (d, J=2.0 Hz, 1H), 7.15 (s, 1H), 6.84 (d, J=8.4 Hz, 1H), 6.38 (d, J=2.8 Hz, 1H), 3.72 (s, 3H); ESI-MS 146.08 m/z (MH⁺).

### Other Examples

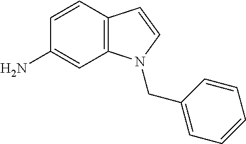 [SMILES: Nc1ccc2ccn(Cc3ccccc3)c2c1]

### B-2; 1-Benzyl-1H-indol-6-ylamine

1-Benzyl-1H-indol-6-ylamine (B-2) was synthesized following the general scheme above starting from 6-nitroindole and benzyl bromide. Overall yield (˜40%). HPLC ret. time 2.19 min, 10-99% CH₃CN, 5 min run; ESI-MS 223.3 m/z (MH⁺).

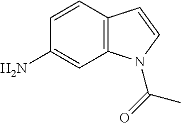

### B-3; 1-(6-Amino-indol-1-yl)-ethanone

1-(6-Amino-indol-1-yl)-ethanone (B-3) was synthesized following the general scheme above starting from 6-nitroindole and acetyl chloride. Overall yield 40%). HPLC ret. time 0.54 min, 10-99% CH₃CN, 5 min run; ESI-MS 175.1 m/z (MH+).

### Example 2

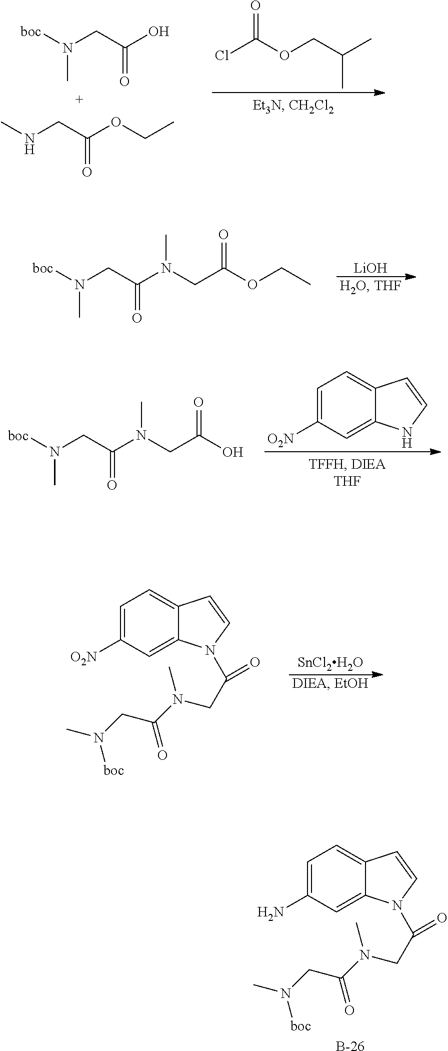

### {[2-(tert-Butoxycarbonyl-methyl-amino)-acetyl]-methyl-amino}-acetic acid ethyl ester

To a stirred solution of (tert-butoxycarbonyl-methyl-amino)-acetic acid (37 g, 0.2 mol) and Et₃N (60.6 g, 0.6 mol) in CH₂Cl₂ (300 mL) was added isobutyl chloroformate (27.3 g, 0.2 mmol) dropwise at −20° C. under argon. After stirring for 0.5 h, methylamino-acetic acid ethyl ester hydrochloride (30.5 g, 129 mmol) was added dropwise at −20° C. The mixture was allowed to warm to room temperature (c.a. 1 h) and quenched with water (500 mL). The organic layer was separated, washed with 10% citric acid solution, dried over Na₂SO₄, filtered and concentrated. The residue was purified by column chromatography (petroleum ether/EtOAc 1:1) to give {[2-(tert-butoxycarbonyl-methyl-amino)-acetyl]methyl-amino}-acetic acid ethyl ester (12.5 g, 22%).

### {[2-(tert-Butoxycarbonyl-methyl-amino)-acetyl]-methyl-amino}-acetic acid

A suspension of {[2-(tert-butoxycarbonyl-methyl-amino)-acetyl]-methyl-amino}-acetic acid ethyl ester (12.3 g, 42.7 mmol) and LiOH (8.9 g, 214 mmol) in H₂O (20 mL) and THF (100 mL) was stirred overnight. Volatile solvent was removed under vacuum and the residue was extracted with ether (2×100 mL). The aqueous phase was acidified to pH 3 with dilute HCl solution, and then extracted with CH₂Cl₂ (2×300 mL). The combined organic layers were washed with brine, dried over Na₂SO₄ and concentrated under vacuum to give {[2-(tert-butoxycarbonyl-methyl-amino)-acetyl]-methyl-amino}-acetic acid as a colorless oil (10 g, 90%. ¹H NMR (CDCl₃) δ 7.17 (br s, 1H), 4.14-4.04 (m, 4H), 3.04-2.88 (m, 6H), 1.45-1.41 (m, 9H); ESI-MS 282.9 m/z (M+Na⁺).

### Methyl-({methyl-[2-(6-nitro-indol-1-yl)-2-oxo-ethyl]-carbamoyl}-methyl)-carbamic acid tert-butyl ester

To a mixture of {[2-(tert-butoxycarbonyl-methyl-amino)-acetyl]-methyl-amino}-acetic acid (13.8 g, 53 mmol) and TFFH (21.0 g, 79.5 mmol) in anhydrous THF (125 mL) was added DIEA (27.7 mL, 159 mmol) at room temperature under nitrogen. The solution was stirred at room temperature for 20 min. A solution of 6-nitroindole (8.6 g, 53 mmol) in THF (75 mL) was added and the reaction mixture was heated at 60° C. for 18 h. The solvent was evaporated and the crude mixture was re-partitioned between EtOAc and water. The organic layer was separated, washed with water (×3), dried over Na₂SO₄ and concentrated. Diethyl ether followed by EtOAc was added. The resulting solid was collected via filtration, washed with diethyl ether and air dried to yield methyl-({methyl-[2-(6-nitro-indol-1-yl)-2-oxo-ethyl]-carbamoyl}-methyl)-carbamic acid tert-butyl ester (6.42 g, 30%). ¹H NMR (400 MHz, DMSO-d6) δ 1.37 (m, 9H), 2.78 (m, 3H), 2.95 (d, J=1.5 Hz, 1H), 3.12 (d, J=2.1 Hz, 2H), 4.01 (d, J=13.8 Hz, 0.6H), 4.18 (d, J=12.0 Hz, 1.4H), 4.92 (d, J=3.4 Hz, 1.4H), 5.08 (d, J=11.4 Hz, 0.6H), 7.03 (m, 1H), 7.90 (m, 1H), 8.21 (m, 1H), 8.35 (d, J=3.8 Hz, 1H), 9.18 (m, 1H); HPLC ret. time 3.12 min, 10-99% CH₃CN, 5 min run; ESI-MS 405.5 m/z (MH⁺).

### B-26; ({[2-(6-Amino-indol-1-yl)-2-oxo-ethyl]-methyl-carbamoyl}-methyl)-methyl-carbamic acid tert-butyl ester

A mixture of methyl-({methyl-[2-(6-nitro-indol-1-yl)-2-oxo-ethyl]-carbamoyl}-methyl)-carbamic acid tert-butyl ester (12.4 g, 30.6 mmol), SnCl₂.2H₂O (34.5 g, 153.2 mmol) and DIEA (74.8 mL, 429 mmol) in ethanol (112 mL) was heated to 70° C. for 3 h. Water and EtOAc were added and the mixture was filtered through a short plug of Celite. The organic layer was separated, dried over Na₂SO₄ and concentrated to yield ({[2-(6-Amino-indol-1-yl)-2-oxo-ethyl]-methyl-carbamoyl}-methyl)-methyl-carbamic acid tert-butyl ester (B-26) (11.4 g, quant.). HPLC ret. time 2.11 min, 10-99% CH₃CN, 5 min run; ESI-MS 375.3 m/z (MH⁺).

### 2-Substituted 6-aminoindoles

### Example 1

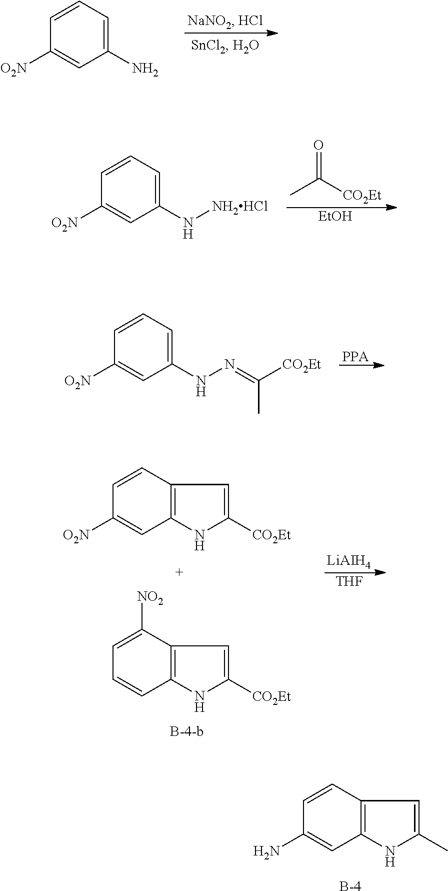

### B-4-a; (3-Nitro-phenyl)-hydrazine hydrochloride salt

3-Nitro-phenylamine (27.6 g, 0.2 mol) was dissolved in a mixture of H₂O (40 mL) and 37% HCl (40 mL). A solution of NaNO₂ (13.8 g, 0.2 mol) in H₂O (60 mL) was added at 0° C., followed by the addition of SnCl₂.H₂O (135.5 g, 0.6 mol) in 37% HCl (100 mL) at that temperature. After stirring at 0° C. for 0.5 h, the solid was isolated via filtration and washed with water to give (3-nitro-phenyl)-hydrazine hydrochloride salt (B-4-a) (27.6 g, 73%).

### 2-[(3-Nitro-phenyl)-hydrazono]-propionic acid ethyl ester

(3-Nitro-phenyl)-hydrazine hydrochloride salt (B-4-a) (30.2 g, 0.16 mol) and 2-oxo-propionic acid ethyl ester (22.3 g, 0.19 mol) was dissolved in ethanol (300 mL). The mixture was stirred at room temperature for 4 h. The solvent was evaporated under reduced pressure to give 2-[(3-nitro-phenyl)-hydrazono]-propionic acid ethyl ester, which was used directly in the next step.

### B-4-b; 4-Nitro-1H-indole-2-carboxylic acid ethyl ester and 6-Nitro-1H-indole-2-carboxylic acid ethyl ester

2-[(3-Nitro-phenyl)-hydrazono]-propionic acid ethyl ester from the preceding step was dissolved in toluene (300 mL). PPA (30 g) was added. The mixture was heated at reflux overnight and then cooled to room temperature. The solvent was removed to give a mixture of 4-nitro-1H-indole-2-carboxylic acid ethyl ester and 6-nitro-1H-indole-2-carboxylic acid ethyl ester (B-4-b) (15 g, 40%).

### B-4; 2-Methyl-1H-indol-6-ylamine

To a suspension of LiAlH₄ (7.8 g, 0.21 mol) in THF (300 mL) was added dropwise a mixture of 4-nitro-1H-indole-2-carboxylic acid ethyl ester and 6-nitro-1H-indole-2-carboxylic acid ethyl ester (B-4-b) (6 g, 25.7 mmol) in THF (50 mL) at 0° C. under N₂. The mixture was heated at reflux overnight and then cooled to 0° C. H₂O (7.8 mL) and 10% NaOH (7.8 mL) were added to the mixture at 0° C. The insoluble solid was removed via filtration. The filtrate was dried over Na₂SO₄, filtered and concentrated under reduced pressure. The crude residue was purified by column chromatography to afford 2-methyl-1H-indol-6-ylamine (B-4) (0.3 g, 8%). ¹H NMR (CDCl₃) δ 7.57 (br s, 1H), 7.27 (d, J=8.8 Hz, 1H), 6.62 (s, 1H), 6.51-6.53 (m, 1H), 6.07 (s, 1H), 3.59-3.25 (br s, 2H), 2.37 (s, 3H); ESI-MS 147.2 m/z (MH⁺).

### Example 2

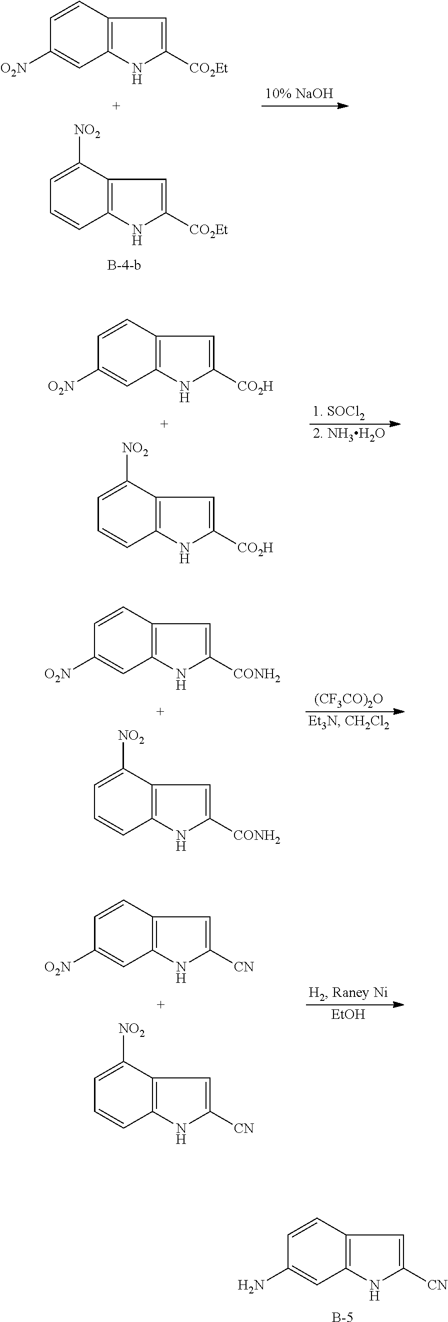

### 6-Nitro-1H-indole-2-carboxylic acid and 4-Nitro-1H-indole-2-carboxylic acid

A mixture of 4-nitro-1H-indole-2-carboxylic acid ethyl ester and 6-nitro-1H-indole-2-carboxylic acid ethyl ester (B-4-b) (0.5 g, 2.13 mmol) in 10% NaOH (20 mL) was heated at reflux overnight and then cooled to room temperature. The mixture was extracted with ether. The aqueous phase was separated and acidified with HCl to pH 1-2. The resulting solid was isolated via filtration to give a mixture of 6-nitro-1H-indole-2-carboxylic acid and 4-nitro-1H-indole-2-carboxylic acid (0.3 g, 68%).

### 6-Nitro-1H-indole-2-carboxylic acid amide and 4-Nitro-1H-indole-2-carboxylic acid amide

A mixture of 6-nitro-1H-indole-2-carboxylic acid and 4-nitro-1H-indole-2-carboxylic acid (12 g, 58 mmol) and SOCl₂ (50 mL, 64 mmol) in benzene (150 mL) was refluxed for 2 h. The benzene and excessive SOCl₂ was removed under reduced pressure. The residue was dissolved in CH₂Cl₂ (250 mL). NH₄OH (21.76 g, 0.32 mol) was added dropwise at 0° C. The mixture was stirred at room temperature for 1 h. The resulting solid was isolated via filtration to give a crude mixture of 6-nitro-1H-indole-2-carboxylic acid amide and 4-nitro-1H-indole-2-carboxylic acid amide (9 g, 68%), which was used directly in the next step.

### 6-Nitro-1H-indole-2-carbonitrile and 4-Nitro-1H-indole-2-carbonitrile

A mixture of 6-nitro-1H-indole-2-carboxylic acid amide and 4-nitro-1H-indole-2-carboxylic acid amide (5 g, 24 mmol) was dissolved in CH₂Cl₂ (200 mL). Et₃N (24.24 g, 0.24 mol) was added, followed by the addition of (CF₃CO)₂O (51.24 g, 0.24 mol) at room temperature. The mixture was stirred for 1 h and poured into water (100 mL). The organic layer was separated. The aqueous layer was extracted with EtOAc (100 mL×3). The combined organic layers were dried over Na₂SO₄, filtered and concentrated under reduced pressure. The crude residue was purified by column chromatography to give a mixture of 6-nitro-1H-indole-2-carbonitrile and 4-nitro-1H-indole-2-carbonitrile (2.5 g, 55%).

### B-5; 6-Amino-1H-indole-2-carbonitrile

A mixture of 6-nitro-1H-indole-2-carbonitrile and 4-nitro-1H-indole-2-carbonitrile (2.5 g, 13.4 mmol) and Raney Ni (500 mg) in EtOH (50 mL) was stirred at room temperature under H₂ (1 atm) for 1 h. Raney Ni was filtered off. The filtrate was evaporated under reduced pressure and purified by column chromatography to give 6-amino-1H-indole-2-carbonitrile (B-5) (1 g, 49%). ¹H NMR (DMSO-d₆) δ 12.75 (br s, 1H), 7.82 (d, J=8 Hz, 1H), 7.57 (s, 1H), 7.42 (s, 1H), 7.15 (d, J=8 Hz, 1H); ESI-MS 158.2 m/z (MH⁺).

### Example 3

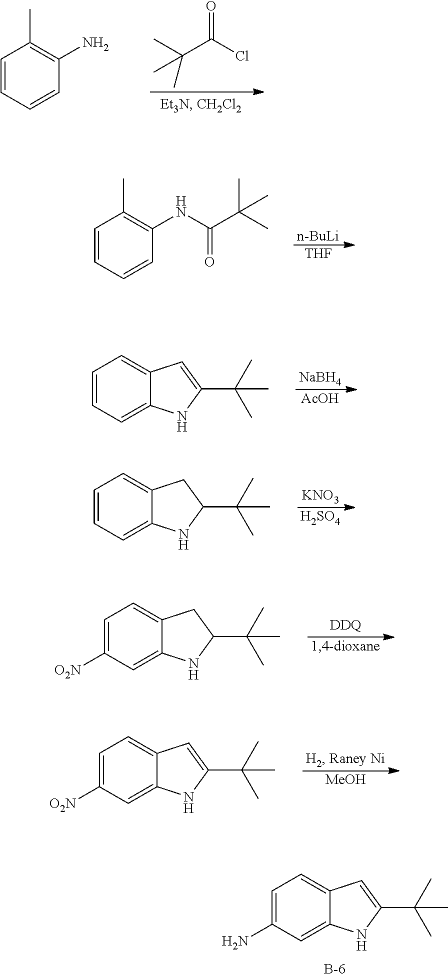

### 2,2-Dimethyl-N-o-tolyl-propionamide

To a solution of o-tolylamine (21.4 g, 0.20 mol) and Et₃N (22.3 g, 0.22 mol) in CH₂Cl₂ was added 2,2-dimethyl-propionyl chloride (25.3 g, 0.21 mol) at 10° C. The mixture was stirred overnight at room temperature, washed with aq. HCl (5%, 80 mL), saturated NaHCO₃ solution and brine, dried over Na₂SO₄ and concentrated under vacuum to give 2,2-dimethyl-N-o-tolyl-propionamide (35.0 g, 92%).

### 2-tert-Butyl-1H-indole

To a solution of 2,2-dimethyl-N-o-tolyl-propionamide (30.0 g, 159 mmol) in dry THF (100 mL) was added dropwise n-BuLi (2.5 M, in hexane, 190 mL) at 15° C. The mixture was stirred overnight at 15° C., cooled in an ice-water bath and treated with saturated NH₄Cl solution. The organic layer was separated and the aqueous layer was extracted with ethyl acetate. The combined organic layers were dried over anhydrous Na₂SO₄, filtered, and concentrated in vacuum. The residue was purified by column chromatography to give 2-tert-butyl-1H-indole (23.8 g, 88%).

### 2-tert-Butyl-2,3-dihydro-1H-indole

To a solution of 2-tert-butyl-1H-indole (5.0 g, 29 mmol) in AcOH (20 mL) was added NaBH₄ at 10° C. The mixture was stirred for 20 min at 10° C., treated dropwise with H₂O under ice cooling, and extracted with ethyl acetate. The combined organic layers were dried over anhydrous Na₂SO₄, filtered, and concentrated under vacuum to give a mixture of starting material and 2-tert-butyl-2,3-dihydro-1H-indole (4.9 g), which was used directly in the next step.

### 2-tert-Butyl-6-nitro-2,3-dihydro-1H-indole

To a solution of the mixture of 2-tert-butyl-2,3-dihydro-1H-indole and 2-tert-butyl-1H-indole (9.7 g) in H₂SO₄ (98%, 80 mL) was slowly added KNO₃ (5.6 g, 55.7 mmol) at 0° C. The reaction mixture was stirred at room temperature for 1 h, carefully poured into cracked ice, basified with Na₂CO₃ to pH ˜8 and extracted with ethyl acetate. The combined extracts were washed with brine, dried over anhydrous Na₂SO₄ and concentrated under vacuum. The residue was purified by column chromatography to give 2-tert-butyl-6-nitro-2,3-dihydro-1H-indole (4.0 g, 32% over 2 steps).

### 2-tert-Butyl-6-nitro-1H-indole

To a solution of 2-tert-butyl-6-nitro-2,3-dihydro-1H-indole (2.0 g, 9.1 mmol) in 1,4-dioxane (20 mL) was added DDQ at room temperature. After refluxing for 2.5 h, the mixture was filtered and the filtrate was concentrated under vacuum. The residue was purified by column chromatography to give 2-tert-butyl-6-nitro-1H-indole (1.6 g, 80%).

### B-6; 2-tert-Butyl-1H-indol-6-ylamine

To a solution of 2-tert-butyl-6-nitro-1H-indole (1.3 g, 6.0 mmol) in MeOH (10 mL) was added Raney Ni (0.2 g). The mixture was stirred at room temperature under H₂ (1 atm) for 3 h. The reaction mixture was filtered and the filtrate was concentrated. The residue was washed with petroleum ether to give 2-tert-butyl-1H-indol-6-ylamine (B-6) (1.0 g, 89%). ¹H NMR (DMSO-d₆) δ 10.19 (s, 1H), 6.99 (d, J=8.1 Hz, 1H), 6.46 (s, 1H), 6.25 (dd, J=1.8, 8.1 Hz, 1H), 5.79 (d, J=1.8 Hz, 1H), 4.52 (s, 2H), 1.24 (s, 9H); ESI-MS 189.1 m/z (MH⁺).

### 3-Substituted 6-aminoindoles

### Example 1

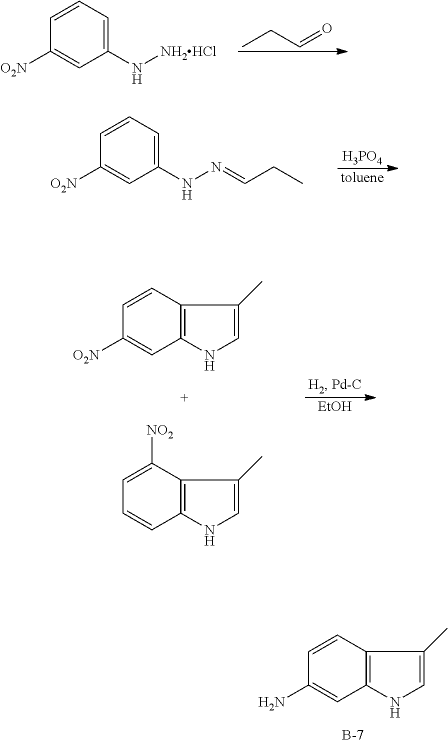

### N-(3-Nitro-phenyl)-N′-propylidene-hydrazine

Sodium hydroxide solution (10%, 15 mL) was added slowly to a stirred suspension of (3-nitro-phenyl)-hydrazine hydrochloride salt (B-4-a) (1.89 g, 10 mmol) in ethanol (20 mL) until pH 6. Acetic acid (5 mL) was added to the mixture followed by propionaldehyde (0.7 g, 12 mmol). After stirring for 3 h at room temperature, the mixture was poured into ice-water and the resulting precipitate was isolated via filtration, washed with water and dried in air to obtain N-(3-nitro-phenyl)-N′-propylidene-hydrazine, which was used directly in the next step.

### 3-Methyl-4-nitro-1H-indole and 3-Methyl-6-nitro-1H-indole

A mixture of N-(3-nitro-phenyl)-N′-propylidene-hydrazine dissolved in 85% H₃PO₄ (20 mL) and toluene (20 mL) was heated at 90-100° C. for 2 h. After cooling, toluene was removed under reduced pressure. The resultant oil was basified with 10% NaOH to pH 8. The aqueous layer was extracted with EtOAc (100 mL×3). The combined organic layers were dried, filtered and concentrated under reduced pressure to afford a mixture of 3-methyl-4-nitro-1H-indole and 3-methyl-6-nitro-1H-indole (1.5 g, 86% over two steps), which was used directly in the next step.

### B-7; 3-Methyl-1H-indol-6-ylamine

A mixture of 3-methyl-4-nitro-1H-indole and 3-methyl-6-nitro-1H-indole (3 g, 17 mol) and 10% Pd—C (0.5 g) in ethanol (30 mL) was stirred overnight under H₂ (1 atm) at room temperature. Pd—C was filtered off and the filtrate was concentrated under reduced pressure. The residue was purified by column chromatography to give 3-methyl-1H-indol-6-ylamine (B-7) (0.6 g, 24%). ¹H NMR (CDCl₃) δ 7.59 (br s, 1H), 7.34 (d, J=8.0 Hz, 1H), 6.77 (s, 1H), 6.64 (s, 1H), 6.57 (m, 1H), 3.57 (br s, 2H), 2.28 (s, 3H); ESI-MS 147.2 m/z (MH⁺).

### Example 2

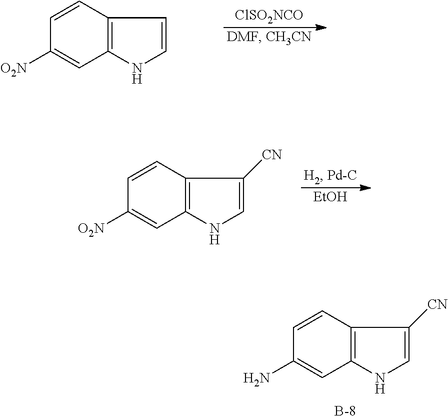

### 6-Nitro-1H-indole-3-carbonitrile

To a solution of 6-nitroindole (4.86 g 30 mmol) in DMF (24.3 mL) and CH₃CN (243 mL) was added dropwise a solution of ClSO₂NCO (5 mL, 57 mmol) in CH₃CN (39 mL) at 0° C. After addition, the reaction was allowed to warm to room temperature and stirred for 2 h. The mixture was poured into ice-water, basified with sat. NaHCO₃ solution to pH 7-8 and extracted with ethyl acetate. The organic layer was washed with brine, dried over Na₂SO₄ and concentrated to give 6-nitro-1H-indole-3-carbonitrile (4.6 g, 82%).

### B-8; 6-Amino-1H-indole-3-carbonitrile

A suspension of 6-nitro-1H-indole-3-carbonitrile (4.6 g, 24.6 mmol) and 10% Pd—C (0.46 g) in EtOH (50 mL) was stirred under H₂ (1 atm) at room temperature overnight. After filtration, the filtrate was concentrated and the residue was purified by column chromatography (Pet. Ether/EtOAc=3/1) to give 6-amino-1H-indole-3-carbonitrile (B-8) (1 g, 99%) as a pink powder. ¹H NMR (DMSO-d₆) δ 11.51 (s, 1H), 7.84 (d, J=2.4 Hz, 1H), 7.22 (d, J=8.4 Hz, 1H), 6.62 (s, 1H), 6.56 (d, J=8.4 Hz, 1H), 5.0 (s, 2H); ESI-MS 157.1 m/z (MH⁺).

### Example 3

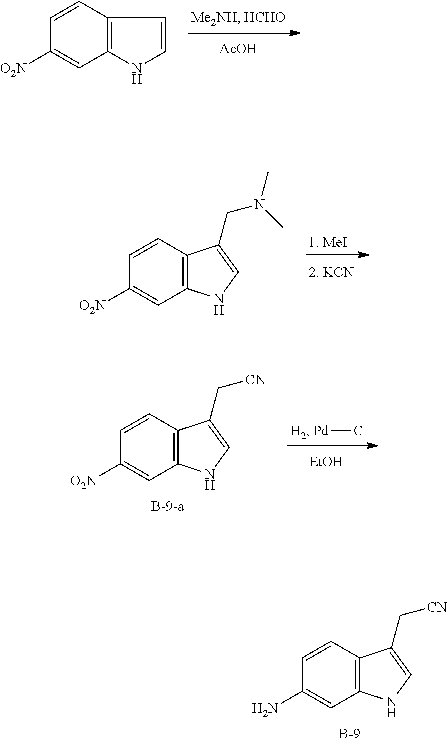

### Dimethyl-(6-nitro-1H-indol-3-ylmethyl)-amine

A solution of dimethylamine (25 g, 0.17 mol) and formaldehyde (14.4 mL, 0.15 mol) in acetic acid (100 mL) was stirred at 0° C. for 30 min. To this solution was added 6-nitro-1H-indole (20 g, 0.12 mol). After stirring for 3 days at room temperature, the mixture was poured into 15% aq. NaOH solution (500 mL) at 0° C. The precipitate was collected via filtration and washed with water to give dimethyl-(6-nitro-1H-indol-3-ylmethyl)-amine (23 g, 87%).

### B-9-a; (6-Nitro-1H-indol-3-yl)-acetonitrile

To a mixture of DMF (35 mL) and MeI (74.6 g, 0.53 mol) in water (35 mL) and THF (400 mL) was added dimethyl-(6-nitro-1H-indol-3-ylmethyl)-amine (23 g, 0.105 mol). After the reaction mixture was refluxed for 10 min, potassium cyanide (54.6 g, 0.84 mol) was added and the mixture was kept refluxing overnight. The mixture was then cooled to room temperature and filtered. The filtrate was washed with brine (300 ml, ×3), dried over Na₂SO₄, filtered and concentrated. The residue was purified by column chromatography to give (6-nitro-1H-indol-3-yl)-acetonitrile (B-9-a) (7.5 g, 36%).

### B-9; (6-Amino-1H-indol-3-yl)-acetonitrile

A mixture of (6-nitro-1H-indol-3-yl)-acetonitrile (B-9-a) (1.5 g, 74.5 mml) and 10% Pd—C (300 mg) in EtOH (50 mL) was stirred at room temperature under H₂ (1 atm) for 5 h. Pd—C was removed via filtration and the filtrate was evaporated to give (6-amino-1H-indol-3-yl)-acetonitrile (B-9) (1.1 g, 90%). ¹H NMR (DMSO-d₆) δ 10.4 (br s, 1H), 7.18 (d, J=8.4 Hz, 1H), 6.94 (s, 1H), 6.52 (s, 1H), 6.42 (dd, J=8.4, 1.8 Hz, 1H), 4.76 (s, 2H), 3.88 (s, 2H); ESI-MS 172.1 m/z (MH⁺).

### Example 4

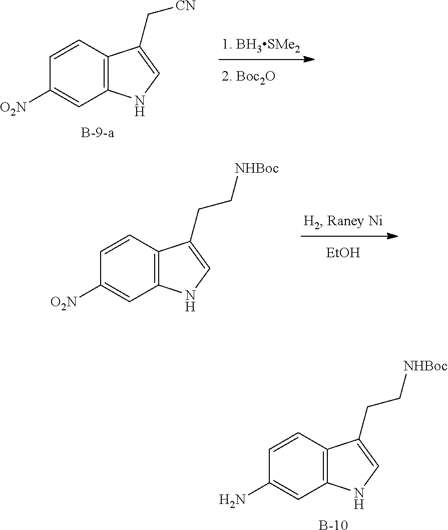

### [2-(6-Nitro-1H-indol-3-yl)-ethyl]-carbamic acid tert-butyl ester

To a solution of (6-nitro-1H-indol-3-yl)-acetonitrile (B-9-a) (8.6 g, 42.8 mmol) in dry THF (200 mL) was added a solution of 2 M borane-dimethyl sulfide complex in THF (214 mL. 0.43 mol) at 0° C. The mixture was heated at reflux overnight under nitrogen. The mixture was then cooled to room temperature and a solution of (Boc)₂O (14 g, 64.2 mmol) and Et₃N (89.0 mL, 0.64 mol) in THF was added. The reaction mixture was kept stirring overnight and then poured into ice-water. The organic layer was separated and the aqueous phase was extracted with EtOAc (200×3 mL). The combined organic layers were washed with water and brine, dried over Na₂SO₄, filtered and concentrated under reduced pressure. The crude was purified by column chromatography to give [2-(6-nitro-1H-indol-3-yl)-ethyl]-carbamic acid tert-butyl ester (5 g, 38%).

### B-10; [2-(6-Amino-1H-indol-3-yl)-ethyl]carbamic acid tert-butyl ester

A mixture of [2-(6-nitro-1H-indol-3-yl)-ethyl]-carbamic acid tert-butyl ester (5 g, 16.4 mmol) and Raney Ni (1 g) in EtOH (100 mL) was stirred at room temperature under H₂ (1 atm) for 5 h. Raney Ni was filtered off and the filtrate was evaporated under reduced pressure. The crude product was purified by column chromatography to give [2-(6-amino-1H-indol-3-yl)-ethyl]carbamic acid tert-butyl ester (B-10) (3 g, 67%). ¹H NMR (DMSO-d₆) δ 10.1 (br s, 1H), 7.11 (d, J=8.4 Hz, 1H), 6.77-6.73 (m, 2H), 6.46 (d, J=1.5 Hz, 1H), 6.32 (dd, J=8.4, 2.1 Hz, 1H), 4.62 (s, 2H), 3.14-3.08 (m, 2H), 2.67-2.62 (m, 2H), 1.35 (s, 9H); ESI-MS 275.8 m/z (MH⁺).

### Example 5

### General Scheme

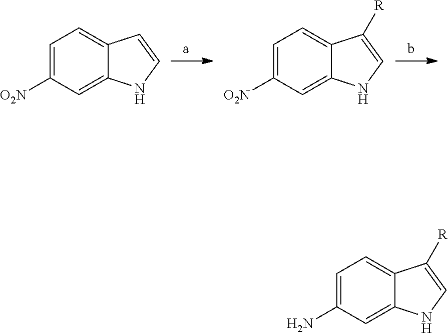

a) RX (X=Br,I), zinc triflate, TBAI, DIEA, toluene; b) H₂, Raney Ni, EtOH or SnCl₂.2H₂O, EtOH.

### Specific Example

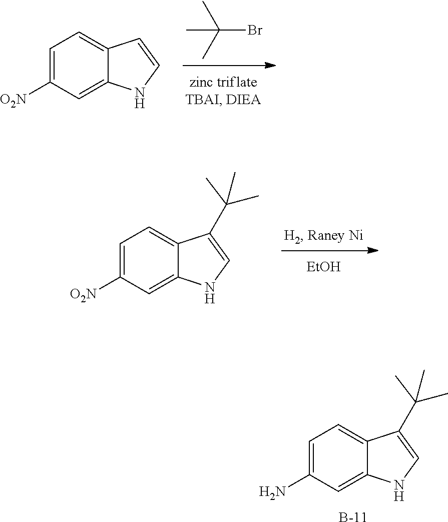

### 3-tert-Butyl-6-nitro-1H-indole

To a mixture of 6-nitroindole (1 g, 6.2 mmol), zinc triflate (2.06 g, 5.7 mmol) and TBAI (1.7 g, 5.16 mmol) in anhydrous toluene (11 mL) was added DIEA (1.47 g, 11.4 mmol) at room temperature under nitrogen. The reaction mixture was stirred for 10 min at 120° C., followed by addition of t-butyl bromide (0.707 g, 5.16 mmol). The resulting mixture was stirred for 45 min at 120° C. The solid was filtered off and the filtrate was concentrated to dryness and purified by column chromatography on silica gel (Pet.Ether./EtOAc 20:1) to give 3-tert-butyl-6-nitro-1H-indole as a yellow solid (0.25 g, 19%). ¹H NMR (CDCl₃) δ 8.32 (d, J=2.1 Hz, 1H), 8.00 (dd, J=2.1, 14.4 Hz, 1H), 7.85 (d, J=8.7 Hz, 1H), 7.25 (s, 1H), 1.46 (s, 9H).

### B-11; 3-tert-Butyl-1H-indol-6-ylamine

A suspension of 3-tert-butyl-6-nitro-1H-indole (3.0 g, 13.7 mmol) and Raney Ni (0.5 g) in ethanol was stirred at room temperature under H₂ (1 atm) for 3 h. The catalyst was filtered off and the filtrate was concentrated to dryness. The residue was purified by column chromatography on silica gel (Pet.Ether./EtOAc 4: 1) to give 3-tert-butyl-1H-indol-6-ylamine (B-11) (2.0 g, 77.3%) as a gray solid. ¹H NMR (CDCl₃): δ 7.58 (m, 2H), 6.73 (d, J=1.2 Hz, 1H), 6.66 (s, 1H), 6.57 (dd, J=0.8, 8.6 Hz, 1H), 3.60 (br s, 2H), 1.42 (s, 9H).

### Other Examples

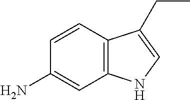

### B-12; 3-Ethyl-1H-indol-6-ylamine

3-Ethyl-1H-indol-6-ylamine (B-12) was synthesized following the general scheme above starting from 6-nitroindole and ethyl bromide. Overall yield (42%). HPLC ret. time 1.95 min, 10-99% CH3CN, 5 min run; ESI-MS 161.3 m/z (MH+).

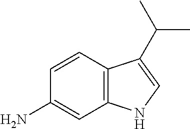

### B-13; 3-Isopropyl-1H-indol-6-ylamine

3-Isopropyl-1H-indol-6-ylamine (B-13) was synthesized following the general scheme above starting from 6-nitroindole and isopropyl iodide. Overall yield (17%). HPLC ret. time 2.06 min, 10-99% CH₃CN, 5 min run; ESI-MS 175.2 m/z (MH⁺).

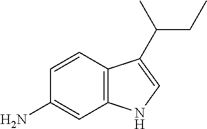

### B-14; 3-sec-Butyl-1H-indol-6-ylamine

3-sec-Butyl-1H-indol-6-ylamine (B-14) was synthesized following the general scheme above starting from 6-nitroindole and 2-bromobutane. Overall yield (20%). HPLC ret. time 2.32 min, 10-99% CH₃CN, 5 min run; ESI-MS 189.5 m/z (MH⁺).

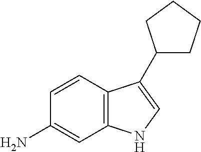 [SMILES: Nc1ccc2c(C3CCCC3)c[nH]c2c1]

### B-15; 3-Cyclopentyl-1H-indol-6-ylamine

3-Cyclopentyl-1H-indol-6-ylamine (B-15) was synthesized following the general scheme above starting from 6-nitroindole and iodo-cyclopentane. Overall yield (16%). HPLC ret. time 2.39 min, 10-99% CH₃CN, 5 min run; ESI-MS 201.5 m/z (MH⁺).

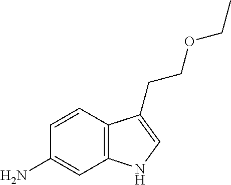

### B-16; 3-(2-Ethoxy-ethyl)-1H-indol-6-ylamine

3-(2-Ethoxy-ethyl)-1H-indol-6-ylamine (B-16) was synthesized following the general scheme above starting from 6-nitroindole and 1-bromo-2-ethoxy-ethane. Overall yield (15%). HPLC ret. time 1.56 min, 10-99% CH₃CN, 5 min run; ESI-MS 205.1 m/z (MH⁺).

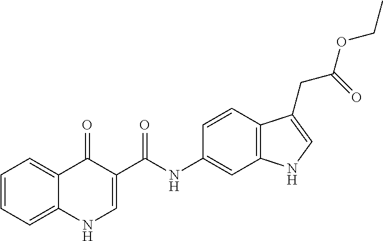 [SMILES: CCOC(=O)Cc1c[nH]c2cc(NC(=O)c3c[nH]c4ccccc4c3=O)ccc12]

### B-17; (6-Amino-1H-indol-3-yl)-acetic acid ethyl ester

(6-Amino-1H-indol-3-yl)-acetic acid ethyl ester (B-17) was synthesized following the general scheme above starting from 6-nitroindole and iodo-acetic acid ethyl ester. Overall yield (24%). HPLC ret. time 0.95 min, 10-99% CH₃CN, 5 min run; ESI-MS 219.2 m/z (MH⁺).

### 4-Substituted 6-aminoindole

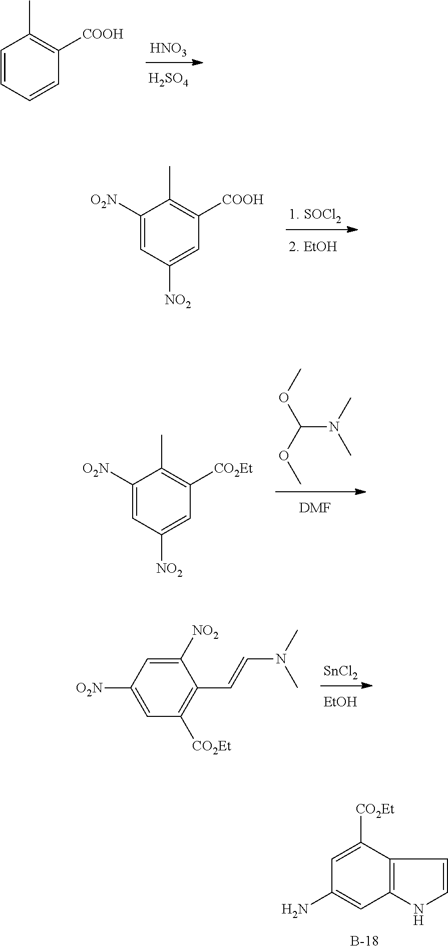

### 2-Methyl-3,5-dinitro-benzoic acid

To a mixture of HNO₃ (95%, 80 mL) and H₂SO₄ (98%, 80 mL) was slowly added 2-methylbenzoic acid (50 g, 0.37 mol) at 0° C. After addition, the reaction mixture was stirred for 1.5 h while keeping the temperature below 30° C., poured into ice-water and stirred for 15 min. The resulting precipitate was collected via filtration and washed with water to give 2-methyl-3,5-dinitro-benzoic acid (70 g, 84%).

### 2-Methyl-3,5-dinitro-benzoic acid ethyl ester

A mixture of 2-methyl-3,5-dinitro-benzoic acid (50 g, 0.22 mol) in SOCl₂ (80 mL) was heated at reflux for 4 h and then was concentrated to dryness. CH₂Cl₂ (50 mL) and EtOH (80 mL) were added. The mixture was stirred at room temperature for 1 h, poured into ice-water and extracted with EtOAc (3×100 mL). The combined extracts were washed with sat. Na₂CO₃ (80 mL), water (2×100 mL) and brine (100 mL), dried over Na₂SO₄ and concentrated to dryness to give 2-methyl-3,5-dinitro-benzoic acid ethyl ester (50 g, 88%).

### 2-(2-Dimethylamino-vinyl)-3,5-dinitro-benzoic acid ethyl ester

A mixture of 2-methyl-3,5-dinitro-benzoic acid ethyl ester (35 g, 0.14 mol) and dimethoxymethyl-dimethyl-amine (32 g, 0.27 mol) in DMF (200 mL) was heated at 100° C. for 5 h. The mixture was poured into ice-water. The precipitate was collected via filtration and washed with water to give 2-(2-dimethylamino-vinyl)-3,5-dinitro-benzoic acid ethyl ester (11.3 g, 48%).

### B-18; 6-Amino-1H-indole-4-carboxylic acid ethyl ester

A mixture of 2-(2-dimethylamino-vinyl)-3,5-dinitro-benzoic acid ethyl ester (11.3 g, 0.037 mol) and SnCl₂ (83 g. 0.37 mol) in ethanol was heated at reflux for 4 h. The mixture was concentrated to dryness and the residue was poured into water and basified with sat. Na₂CO₃ solution to pH 8. The precipitate was filtered off and the filtrate was extracted with ethyl acetate (3×100 mL). The combined extracts were washed with water (2×100 mL) and brine (150 mL), dried over Na₂SO₄ and concentrated to dryness. The residue was purified by column chromatography on silica gel to give 6-amino-1H-indole-4-carboxylic acid ethyl ester (B-18) (3 g, 40%). ¹H NMR (DMSO-d₆) δ 10.76 (br s, 1H), 7.11-7.14 (m, 2H), 6.81-6.82 (m, 1H), 6.67-6.68 (m, 1H), 4.94 (br s, 2H), 4.32-4.25 (q, J=7.2 Hz, 2H), 1.35-1.31 (t, J=7.2, 3H). ESI-MS 205.0 m/z (MH⁺).

### 5-Substituted 6-aminoindoles

### Example 1

### General Scheme

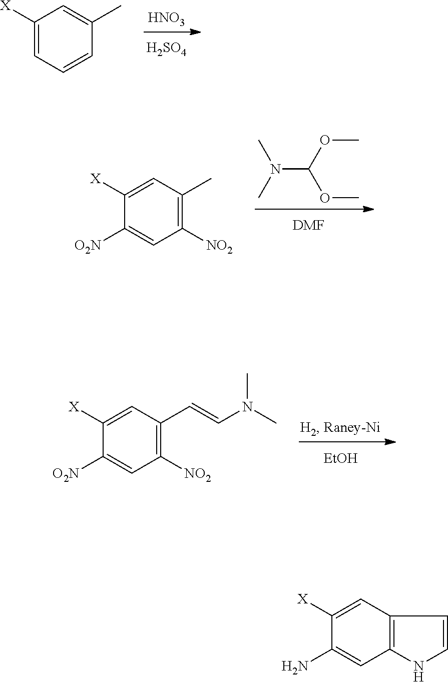

### Specific Example

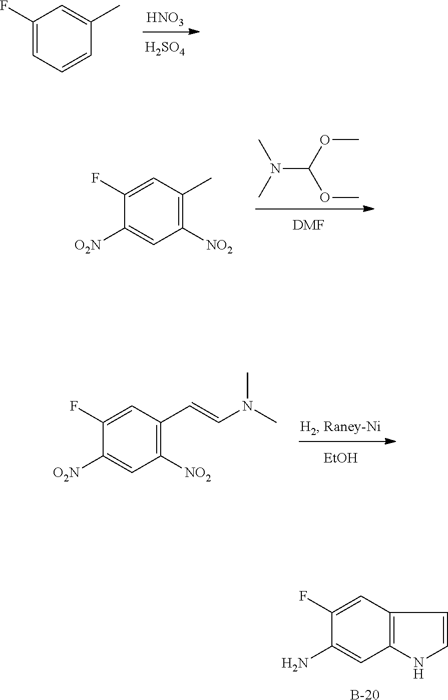

### 1-Fluoro-5-methyl-2,4-dinitro-benzene

To a stirred solution of HNO₃ (60 mL) and H₂SO₄ (80 mL), cooled in an ice bath, was added 1-fluoro-3-methyl-benzene (27.5 g, 25 mmol) at such a rate that the temperature did not rise over 35° C. The mixture was allowed to stir for 30 min at room temperature and poured into ice water (500 mL). The resulting precipitate (a mixture of the desired product and 1-fluoro-3-methyl-2,4-dinitro-benzene, approx. 7:3) was collected via filtration and purified by recrystallization from 50 mL isopropyl ether to give 1-fluoro-5-methyl-2,4-dinitro-benzene as a white solid (18 g, 36%).

### [2-(5-Fluoro-2,4-dinitro-phenyl)-vinyl]-dimethyl-amine

A mixture of 1-fluoro-5-methyl-2,4-dinitro-benzene (10 g, 50 mmol), dimethoxymethyl-dimethylamine (11.9 g, 100 mmol) and DMF (50 mL) was heated at 100° C. for 4 h. The solution was cooled and poured into water. The red precipitate was collected via filtration, washed with water adequately and dried to give [2-(5-fluoro-2,4-dinitro-phenyl)-vinyl]dimethyl-amine (8 g, 63%).

### B-20; 5-Fluoro-1H-indol-6-ylamine

A suspension of [2-(5-fluoro-2,4-dinitro-phenyl)-vinyl]-dimethyl-amine (8 g, 31.4 mmol) and Raney Ni (8 g) in EtOH (80 mL) was stirred under H₂ (40 psi) at room temperature for 1 h. After filtration, the filtrate was concentrated and the residue was purified by chromatography (Pet.Ether/EtOAc=5/1) to give 5-fluoro-1H-indol-6-ylamine (B-20) as a brown solid (1 g, 16%). ¹H NMR (DMSO-d₆) δ 10.56 (br s, 1H), 7.07 (d, J=12 Hz, 1H), 7.02 (m, 1H), 6.71 (d, J=8 Hz, 1H), 6.17 (s, 1H), 3.91 (br s, 2H); ESI-MS 150.1 m/z (MH⁺).

### Other Examples

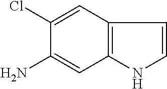 [SMILES: Nc1cc2[nH]ccc2cc1Cl]

### B-21; 5-Chloro-1H-indol-6-ylamine

5-Chloro-1H-indol-6-ylamine (B-21) was synthesized following the general scheme above starting from 1-chloro-3-methyl-benzene. Overall yield (7%). ¹H NMR (CDCl₃) δ. 7.85 (br s, 1H), 7.52 (s, 1H), 7.03 (s, 1H), 6.79 (s, 1H), 6.34 (s, 1H), 3.91 (br s, 2H); ESI-MS 166.0 m/z (MH⁺).

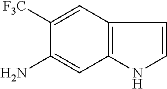 [SMILES: Nc1cc2[nH]ccc2cc1C(F)(F)F]

### B-22; 5-Trifluoromethyl-1H-indol-6-ylamine

5-Trifluoromethyl-1H-indol-6-ylamine (B-22) was synthesized following the general scheme above starting from 1-methyl-3-trifluoromethyl-benzene. Overall yield (2%). ¹H NMR (DMSO-d₆) 10.79 (br s, 1H), 7.55 (s, 1H), 7.12 (s, 1H), 6.78 (s, 1H), 6.27 (s, 1H), 4.92 (s, 2H); ESI-MS 200.8 m/z (MH⁺).

### Example 2

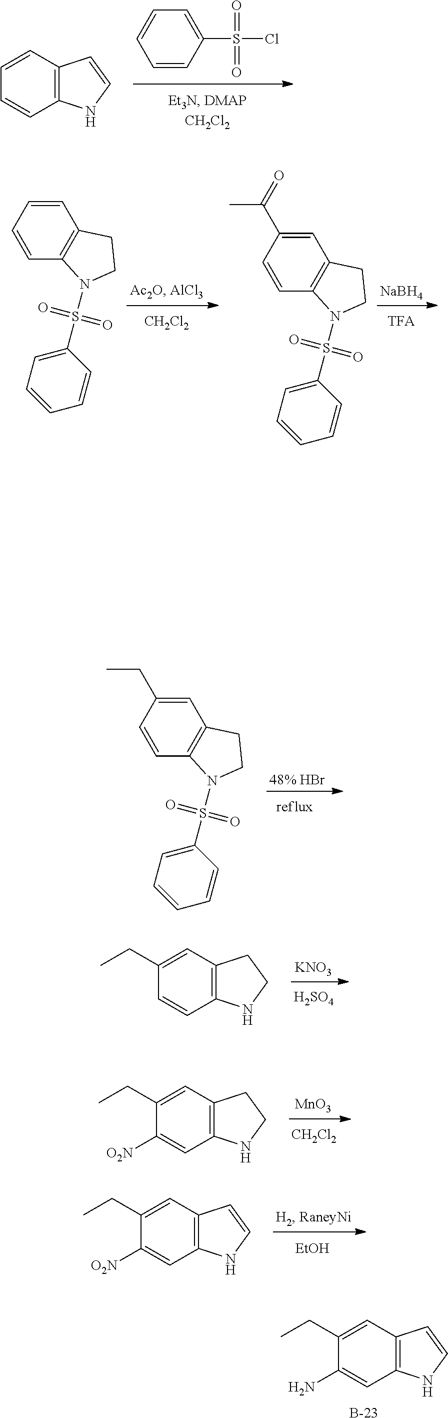

### 1-Benzenesulfonyl-2,3-dihydro-1H-indole

To a mixture of DMAP (1.5 g), benzenesulfonyl chloride (24 g, 136 mmol) and 2,3-dihydro-1H-indole (14.7 g, 124 mmol) in CH₂Cl₂ (200 mL) was added dropwise Et₃N (19 g, 186 mmol) in an ice-water bath. After addition, the mixture was stirred at room temperature overnight, washed with water, dried over Na₂SO₄ and concentrated to dryness under reduced pressure to provide 1-benzenesulfonyl-2,3-dihydro-1H-indole (30.9 g, 96%).

### 1-(1-Benzenesulfonyl-2,3-dihydro-1H-indol-5-yl)-ethanone

To a stirring suspension of AlCl₃ (144 g, 1.08 mol) in CH₂Cl₂ (1070 mL) was added acetic anhydride (54 mL). The mixture was stirred for 15 minutes. A solution of 1-benzenesulfonyl-2,3-dihydro-1H-indole (46.9 g, 0.18 mol) in CH₂Cl₂ (1070 mL) was added dropwise. The mixture was stirred for 5 h and quenched by the slow addition of crushed ice. The organic layer was separated and the aqueous layer was extracted with CH₂Cl₂. The combined organic layers were washed with saturated aqueous NaHCO₃ and brine, dried over Na₂SO₄ and concentrated under vacuum to yield 1-(1-benzenesulfonyl-2,3-dihydro-1H-indol-5-yl)-ethanone (42.6 g, 79%).

### 1-Benzenesulfonyl-5-ethyl-2,3-dihydro-1H-indole

To magnetically stirred TFA (1600 mL) was added at 0° C. sodium borohydride (64 g, 1.69 mol) over 1 h. To this mixture was added dropwise a solution of 1-(1-benzenesulfonyl-2,3-dihydro-1H-indol-5-yl)-ethanone (40 g, 0.13 mol) in TFA (700 mL) over 1 h. The mixture was stirred overnight at 25° C., diluted with H₂O (1600 ml), and basified with sodium hydroxide pellets at 0° C. The organic layer was separated and the aqueous layer was extracted with CH₂Cl₂. The combined organic layers were washed with brine, dried over Na₂SO₄ and concentrated under reduced pressure. The residue was purified by column chromatography on silica gel to give 1-benzenesulfonyl-5-ethyl-2,3-dihydro-1H-indole (16.2 g, 43%).

### 5-Ethyl-2,3-dihydro-1H-indole

A mixture of 1-benzenesulfonyl-5-ethyl-2,3-dihydro-1H-indole (15 g, 0.05 mol) in HBr (48%, 162 mL) was heated at reflux for 6 h. The mixture was basified with sat. NaOH solution to pH 9 and extracted with ethyl acetate. The organic layer was washed with brine, dried over Na₂SO₄ and concentrated under reduced pressure. The residue was purified by column chromatography on silica gel to give 5-ethyl-2,3-dihydro-1H-indole (2.5 g, 32%).

### 5-Ethyl-6-nitro-2,3-dihydro-1H-indole

To a solution of 5-ethyl-2,3-dihydro-1H-indole (2.5 g, 17 mmol) in H₂SO₄ (98%, 20 mL) was slowly added KNO₃ (1.7 g, 17 mmol) at 0° C. After addition, the mixture was stirred at 0-10° C. for 10 min, carefully poured into ice, basified with NaOH solution to pH 9 and extracted with ethyl acetate. The combined extracts were washed with brine, dried over Na₂SO₄ and concentrated to dryness. The residue was purified by column chromatography on silica gel to give 5-ethyl-6-nitro-2,3-dihydro-1H-indole (1.9 g, 58%).

### 5-Ethyl-6-nitro-1H-indole

To a solution of 5-ethyl-6-nitro-2,3-dihydro-1H-indole (1.9 g, 9.9 mmol) in CH₂Cl₂ (30 mL) was added MnO₂ (4 g, 46 mmol). The mixture was stirred at room temperature for 8 h. The solid was filtered off and the filtrate was concentrated to dryness to give crude 5-ethyl-6-nitro-1H-indole (1.9 g, quant.).

### B-23; 5-Ethyl-1H-indol-6-ylamine

A suspension of 5-ethyl-6-nitro-1H-indole (1.9 g, 10 mmol) and Raney Ni (1 g) was stirred under H₂ (1 atm) at room temperature for 2 h. The catalyst was filtered off and the filtrate was concentrated to dryness. The residue was purified by column chromatography on silica gel to give 5-ethyl-1H-indol-6-ylamine (B-23) (760 mg, 48%). ¹H NMR (CDCl₃) δ 7.90 (br s, 1H), 7.41 (s, 1H), 7.00 (s, 1H), 6.78 (s, 2H), 6.39 (s, 1H), 3.39 (br s, 2H), 2.63 (q, J=7.2 Hz, 2H), 1.29 (t, J=6.9 Hz, 3H); ESI-MS 161.1 m/z (MH⁺).

### Example 3

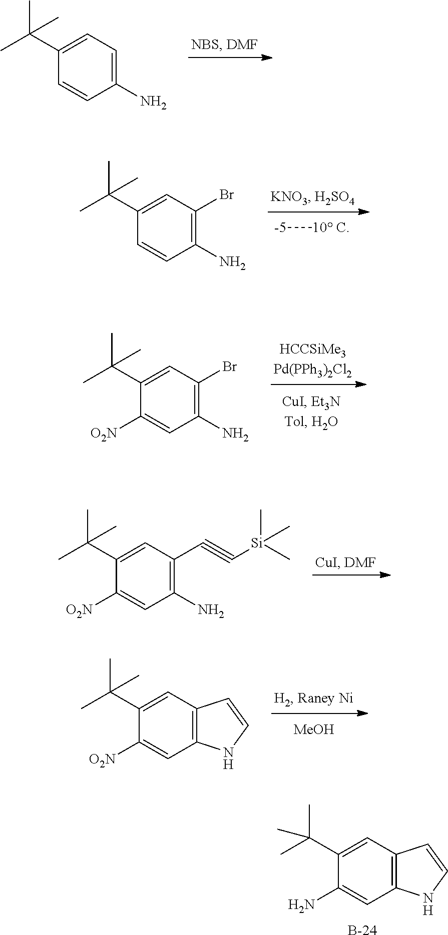

### 2-Bromo-4-tert-butyl-phenylamine

To a solution of 4-tert-butyl-phenylamine (447 g, 3 mol) in DMF (500 mL) was added dropwise NBS (531 g, 3 mol) in DMF (500 mL) at room temperature. Upon completion, the reaction mixture was diluted with water and extracted with EtOAc. The organic layer was washed with water, brine, dried over Na₂SO₄ and concentrated. The crude product was directly used in the next step without further purification.

### 2-Bromo-4-tert-butyl-5-nitro-phenylamine

2-Bromo-4-tert-butyl-phenylamine (162 g, 0.71 mol) was added dropwise to H₂SO₄ (410 mL) at room temperature to yield a clear solution. This clear solution was then cooled down to −5 to −10° C. A solution of KNO₃ (82.5 g, 0.82 mol) in H₂SO₄ (410 mL) was added dropwise while the temperature was maintained between −5 to −10° C. Upon completion, the reaction mixture was poured into ice/water and extracted with EtOAc. The combined organic layers were washed with 5% Na₂CO₃ and brine, dried over Na₂SO₄ and concentrated. The residue was purified by a column chromatography (EtOAc/petroleum ether 1/10) to give 2-bromo-4-tert-butyl-5-nitro-phenylamine as a yellow solid (152 g, 78%).

### 4-tert-Butyl-5-nitro-2-trimethylsilanylethynyl-phenylamine

To a mixture of 2-bromo-4-tert-butyl-5-nitro-phenylamine (27.3 g, 100 mmol) in toluene (200 mL) and water (100 mL) was added Et₃N (27.9 mL, 200 mmol), Pd(PPh₃)₂Cl₂ (2.11 g, 3 mmol), CuI (950 mg, 0.5 mmol) and trimethylsilyl acetylene (21.2 mL, 150 mmol) under a nitrogen atmosphere. The reaction mixture was heated at 70° C. in a sealed pressure flask for 2.5 h., cooled down to room temperature and filtered through a short plug of Celite. The filter cake was washed with EtOAc. The combined filtrate was washed with 5% NH₄OH solution and water, dried over Na₂SO₄ and concentrated. The crude product was purified by column chromatography (0-10% EtOAc/petroleum ether) to provide 4-tert-butyl-5-nitro-2-trimethylsilanylethynyl-phenylamine as a brown viscous liquid (25 g, 81%).

### 5-tert-Butyl-6-nitro-1H-indole

To a solution of 4-tert-butyl-5-nitro-2-trimethylsilanylethynyl-phenylamine (25 g, 86 mmol) in DMF (100 mL) was added CuI (8.2 g, 43 mmol) under a nitrogen atmosphere. The mixture was heated at 135° C. in a sealed pressure flask overnight, cooled down to room temperature and filtered through a short plug of Celite. The filter cake was washed with EtOAc. The combined filtrate was washed with water, dried over Na₂SO₄ and concentrated. The crude product was purified by column chromatography (10-20% EtOAc/Hexane) to provide 5-tert-butyl-6-nitro-1H-indole as a yellow solid (12.9 g, 69%).

### B-24; 5-tert-Butyl-1H-indol-6-ylamine

Raney Ni (3 g) was added to 5-tert-butyl-6-nitro-1H-indole (14.7 g, 67 mmol) in methanol (100 mL). The mixture was stirred under hydrogen (1 atm) at 30° C. for 3 h. The catalyst was filtered off. The filtrate was dried over Na₂SO₄ and concentrated. The crude dark brown viscous oil was purified by column chromatography (10-20% EtOAc/petroleum ether) to give 5-tert-butyl-1H-indol-6-ylamine (B-24) as a gray solid (11 g, 87%). ¹H NMR (300 MHz, DMSO-d6) δ 10.3 (br s, 1H), 7.2 (s, 1H), 6.9 (m, 1H), 6.6 (s, 1H), 6.1 (m, 1H), 4.4 (br s, 2H), 1.3 (s, 9H).

### Example 4

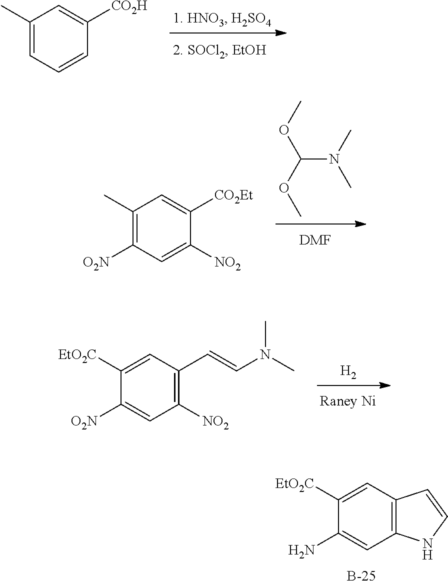

### 5-Methyl-2,4-dinitro-benzoic acid

To a mixture of HNO₃ (95%, 80 mL) and H₂SO₄ (98%, 80 mL) was slowly added 3-methylbenzoic acid (50 g, 0.37 mol) at 0° C. After addition, the mixture was stirred for 1.5 h while maintaining the temperature below 30° C. The mixture was poured into ice-water and stirred for 15 min. The precipitate was collected via filtration and washed with water to give a mixture of 3-methyl-2,6-dinitro-benzoic acid and 5-methyl-2,4-dinitro-benzoic acid (70 g, 84%). To a solution of this mixture in EtOH (150 mL) was added dropwise SOCl₂ (53.5 g, 0.45 mol). The mixture was heated at reflux for 2 h and concentrated to dryness under reduced pressure. The residue was dissolved in EtOAc (100 mL) and extracted with 10% Na₂CO₃ solution (120 mL). The organic layer was found to contain 5-methyl-2,4-dinitro-benzoic acid ethyl ester while the aqueous layer contained 3-methyl-2,6-dinitro-benzoic acid. The organic layer was washed with brine (50 mL), dried over Na₂SO₄ and concentrated to dryness to provide 5-methyl-2,4-dinitro-benzoic acid ethyl ester (20 g, 20%).

### 5-(2-Dimethylamino-vinyl)-2,4-dinitro-benzoic acid ethyl ester

A mixture of 5-methyl-2,4-dinitro-benzoic acid ethyl ester (39 g, 0.15 mol) and dimethoxymethyl-dimethylamine (32 g, 0.27 mol) in DMF (200 mL) was heated at 100° C. for 5 h. The mixture was poured into ice water. The precipitate was collected via filtration and washed with water to afford 5-(2-dimethylamino-vinyl)-2,4-dinitro-benzoic acid ethyl ester (15 g, 28%).

### B-25; 6-Amino-1H-indole-5-carboxylic acid ethyl ester

A mixture of 5-(2-dimethylamino-vinyl)-2,4-dinitro-benzoic acid ethyl ester (15 g, 0.05 mol) and Raney Ni (5 g) in EtOH (500 mL) was stirred under H₂ (50 psi) at room temperature for 2 h. The catalyst was filtered off and the filtrate was concentrated to dryness. The residue was purified by column chromatography on silica gel to give 6-amino-1H-indole-5-carboxylic acid ethyl ester (B-25) (3 g, 30%). ¹H NMR (DMSO-d₆) δ 10.68 (s, 1H), 7.99 (s, 1H), 7.01-7.06 (m, 1H), 6.62 (s, 1H), 6.27-6.28 (m, 1H), 6.16 (s, 2H), 4.22 (q, J=7.2 Hz, 2H), 1.32-1.27 (t, J=7.2 Hz, 3H).

### Example 5

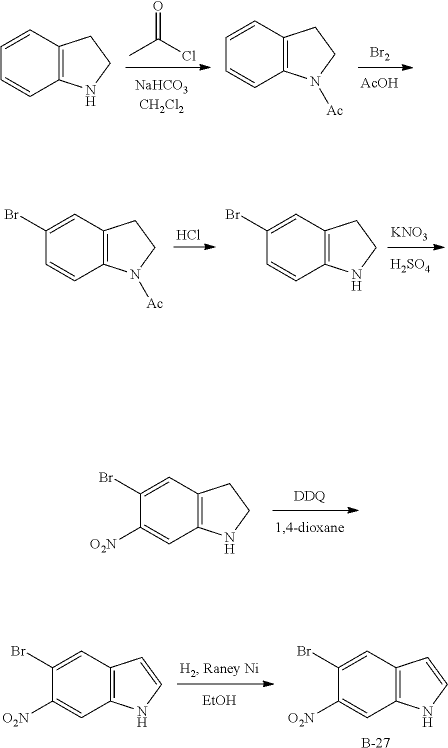

### 1-(2,3-Dihydro-indol-1-yl)-ethanone

To a suspension of NaHCO₃ (504 g, 6.0 mol) and 2,3-dihydro-1H-indole (60 g, 0.5 mol) in CH₂Cl₂ (600 mL) cooled in an ice-water bath, was added dropwise acetyl chloride (78.5 g, 1.0 mol). The mixture was stirred at room temperature for 2 h. The solid was filtered off and the filtrate was concentrated to give 1-(2,3-dihydro-indol-1-yl)-ethanone (82 g, 100%).

### 1-(5-Bromo-2,3-dihydro-indol-1-yl)-ethanone

To a solution of 1-(2,3-dihydro-indol-1-yl)-ethanone (58.0 g, 0.36 mol) in acetic acid (3000 mL) was added Br₂ (87.0 g, 0.54 mol) at 10° C. The mixture was stirred at room temperature for 4 h. The precipitate was collected via filtration to give crude 1-(5-bromo-2,3-dihydro-indol-1-yl)-ethanone (100 g, 96%), which was used directly in the next step.

### 5-Bromo-2,3-dihydro-1H-indole

A mixture of crude 1-(5-bromo-2,3-dihydro-indol-1-yl)-ethanone (100 g, 0.34 mol) in HCl (20%, 1200 mL) was heated at reflux for 6 h. The mixture was basified with Na₂CO₃ to pH 8.5-10 and then extracted with ethyl acetate. The combined organic layers were washed with brine, dried over Na₂SO₄ and concentrated under reduced pressure. The residue was purified by column chromatography on silica gel to give 5-bromo-2,3-dihydro-1H-indole (37 g, 55%).

### 5-Bromo-6-nitro-2,3-dihydro-1H-indole

To a solution of 5-bromo-2,3-dihydro-1H-indole (45 g, 0.227 mol) in H₂SO₄ (98%, 200 mL) was slowly added KNO₃ (23.5 g, 0.23 mol) at 0° C. After addition, the mixture was stirred at 0-10° C. for 4 h, carefully poured into ice, basified with Na₂CO₃ to pH 8 and extracted with ethyl acetate. The combined organic extracts were washed with brine, dried over Na₂SO₄ and concentrated to dryness. The residue was purified by column chromatography on silica gel to give 5-bromo-6-nitro-2,3-dihydro-1H-indole (42 g, 76%).

### 5-Bromo-6-nitro-1H-indole

To a solution of 5-bromo-6-nitro-2,3-dihydro-1H-indole (20 g, 82.3 mmol) in 1,4-dioxane (400 mL) was added DDQ (30 g, 0.13 mol). The mixture was stirred at 80° C. for 2 h. The solid was filtered off and the filtrate was concentrated to dryness. The residue was purified by column chromatography on silica gel to afford 5-bromo-6-nitro-1H-indole (7.5 g, 38%).

### B-27; 5-Bromo-1H-indol-6-ylamine

A mixture of 5-bromo-6-nitro-1H-indole (7.5 g, 31.1 mmol) and Raney Ni (1 g) in ethanol was stirred under H₂ (1 atm) at room temperature for 2 h. The catalyst was filtered off and the filtrate was concentrated to dryness. The residue was purified by column chromatography on silica gel to give 5-bromo-1H-indol-6-ylamine (B-27) (2 g, 30%). ¹H NMR (DMSO-d₆) δ 10.6 (s, 1H), 7.49 (s, 1H), 6.79-7.02 (m, 1H), 6.79 (s, 1H), 6.14-6.16 (m, 1H), 4.81 (s, 2H).

### 7-Substituted 6-aminoindole

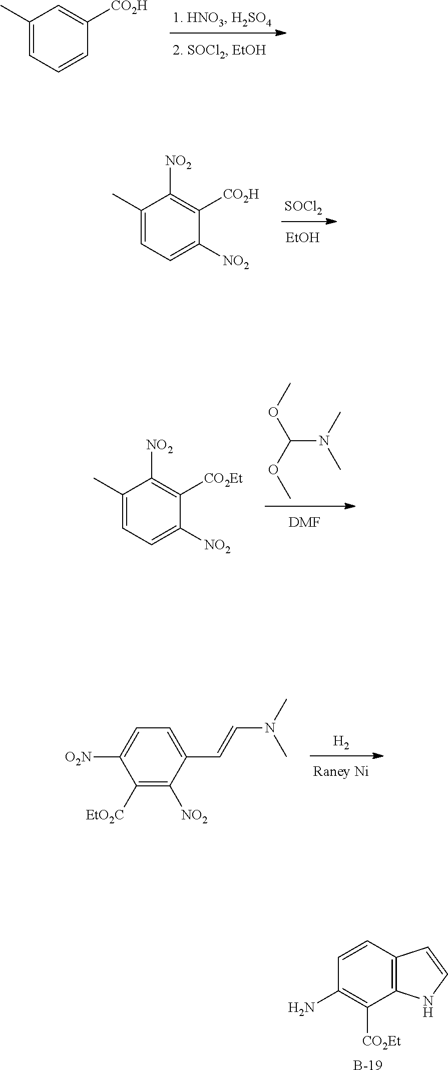

### 3-Methyl-2,6-dinitro-benzoic acid

To a mixture of HNO₃ (95%, 80 mL) and H₂SO₄ (98%, 80 mL) was slowly added 3-methylbenzoic acid (50 g, 0.37 mol) at 0° C. After addition, the mixture was stirred for 1.5 h while maintaining the temperature below 30° C. The mixture was poured into ice-water and stirred for 15 min. The precipitate was collected via filtration and washed with water to give a mixture of 3-methyl-2,6-dinitro-benzoic acid and 5-methyl-2,4-dinitro-benzoic acid (70 g, 84%). To a solution of this mixture in EtOH (150 mL) was added dropwise SOCl₂ (53.5 g, 0.45 mol). The mixture was heated to reflux for 2 h and concentrated to dryness under reduced pressure. The residue was dissolved in EtOAc (100 mL) and extracted with 10% Na₂CO₃ solution (120 mL). The organic layer was found to contain 5-methyl-2,4-dinitro-benzoic acid ethyl ester. The aqueous layer was acidified with HCl to pH 2˜3 and the resulting precipitate was collected via filtration, washed with water and dried in air to give 3-methyl-2,6-dinitro-benzoic acid (39 g, 47%).

### 3-Methyl-2,6-dinitro-benzoic acid ethyl ester

A mixture of 3-methyl-2,6-dinitro-benzoic acid (39 g, 0.15 mol) and SOCl₂ (80 mL) was heated at reflux for 4 h. The excess SOCl₂ was removed under reduced pressure and the residue was added dropwise to a solution of EtOH (100 mL) and Et₃N (50 mL). The mixture was stirred at 20° C. for 1 h and concentrated to dryness. The residue was dissolved in EtOAc (100 mL), washed with Na₂CO₃ (10%, 40 mL×2), water (50 mL×2) and brine (50 mL), dried over Na₂SO₄ and concentrated to give 3-methyl-2,6-dinitro-benzoic acid ethyl ester (20 g, 53%).

### 3-(2-Dimethylamino-vinyl)-2,6-dinitro-benzoic acid ethyl ester

A mixture of 3-methyl-2,6-dinitro-benzoic acid ethyl ester (35 g, 0.14 mol) and dimethoxymethyl-dimethylamine (32 g, 0.27 mol) in DMF (200 mL) was heated at 100° C. for 5 h. The mixture was poured into ice water and the precipitate was collected via filtration and washed with water to give 3-(2-dimethylamino-vinyl)-2,6-dinitro-benzoic acid ethyl ester (25 g, 58%).

### B-19; 6-Amino-1H-indole-7-carboxylic acid ethyl ester

A mixture of 3-(2-dimethylamino-vinyl)-2,6-dinitro-benzoic acid ethyl ester (30 g, 0.097 mol) and Raney Ni (10 g) in EtOH (1000 mL) was stirred under H₂ (50 psi) for 2 h. The catalyst was filtered off, and the filtrate was concentrated to dryness. The residue was purified by column chromatography on silica gel to give 6-amino-1H-indole-7-carboxylic acid ethyl ester (B-19) as an off-white solid (3.2 g, 16%). ¹H NMR (DMSO-d₆) δ 10.38 (s, 1H), 7.44-7.41 (d, J=8.7 Hz, 1H), 6.98 (t, 1H), 6.65 (s, 2H), 6.50-6.46 (m, 1H), 6.27-6.26 (m, 1H), 4.43-4.36 (q, J=7.2 Hz, 2H), 1.35 (t, J=7.2 Hz, 3H).

### Phenols

### Example 1

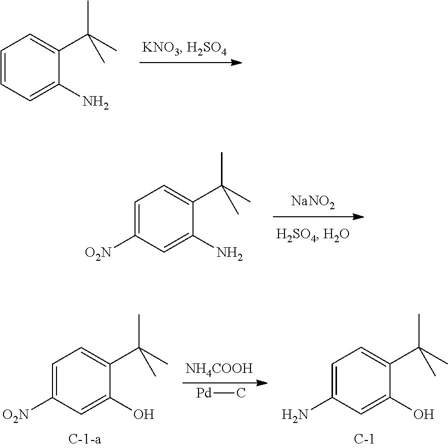

### 2-tert-Butyl-5-nitroaniline

To a cooled solution of sulfuric acid (90%, 50 mL) was added dropwise 2-tert-butyl-phenylamine (4.5 g, 30 mmol) at 0° C. Potassium nitrate (4.5 g, 45 mmol) was added in portions at 0° C. The reaction mixture was stirred at 0-5° C. for 5 min, poured into ice-water and then extracted with EtOAc three times. The combined organic layers were washed with brine and dried over Na₂SO₄. After removal of solvent, the residue was purified by recrystallization using 70% EtOH—H₂O to give 2-tert-butyl-5-nitroaniline (3.7 g, 64%). ¹H NMR (400 MHz, CDCl₃) δ 7.56 (dd, J=8.7, 2.4 Hz, 1H), 7.48 (d, J=2.4 Hz, 1H), 7.36 (d, J=8.7 Hz, 1H), 4.17 (s, 2H), 1.46 (s, 9H); HPLC ret. time 3.27 min, 10-99% CH₃CN, 5 min run; ESI-MS 195.3 m/z (MH⁺).

### C-1-a; 2-tert-Butyl-5-nitrophenol

To a mixture of 2-tert-butyl-5-nitroaniline (1.94 g, 10 mmol) in 40 mL of 15% H₂SO₄ was added dropwise a solution of NaNO₂ (763 mg, 11.0 mmol) in water (3 mL) at 0° C. The resulting mixture was stirred at 0-5° C. for 5 min. Excess NaNO₂ was neutralized with urea, then 5 mL of H₂SO₄—H₂O (v/v 1:2) was added and the mixture was refluxed for 5 min. Three additional 5 mL aliquots of H₂SO₄—H₂O (v/v 1:2) were added while heating at reflux. The reaction mixture was cooled to room temperature and extracted with EtOAc twice. The combined organic layers were washed with brine and dried over MgSO₄. After removal of solvent, the residue was purified by column chromatography (0-10% EtOAc-Hexane) to give 2-tert-butyl-5-nitrophenol (C-1-a) (1.2 g, 62%). ¹H NMR (400 MHz, CDCl₃) δ 7.76 (dd, J=8.6, 2.2 Hz, 1H), 7.58 (d, J=2.1 Hz, 1H), 7.43 (d, J=8.6 Hz, 1H), 5.41 (s, 1H), 1.45 (s, 9H); HPLC ret. time 3.46 min, 10-99% CH₃CN, 5 min run.

C-1; 2-tert-Butyl-5-aminophenol. To a refluxing solution of 2-tert-butyl-5-nitrophenol (C-1-a) (196 mg, 1.0 mmol) in EtOH (10 mL) was added ammonium formate (200 mg, 3.1 mmol), followed by 140 mg of 10% Pd—C. The reaction mixture was refluxed for additional 30 min, cooled to room temperature and filtered through a plug of Celite. The filtrate was concentrated to dryness and purified by column chromatography (20-30% EtOAc-Hexane) to give 2-tert-butyl-5-aminophenol (C-1) (144 mg, 87%). ¹H NMR (400 MHz, DMSO-d₆) δ 8.76 (s, 1H), 6.74 (d, J=8.3 Hz, 1H), 6.04 (d, J=2.3 Hz, 1H), 5.93 (dd, J=8.2, 2.3 Hz, 1H), 4.67 (s, 2H), 1.26 (s, 9H); HPLC ret. time 2.26 min, 10-99% CH₃CN, 5 min run; ESI-MS 166.1 m/z (MH⁺).

### Example 2

### General Scheme

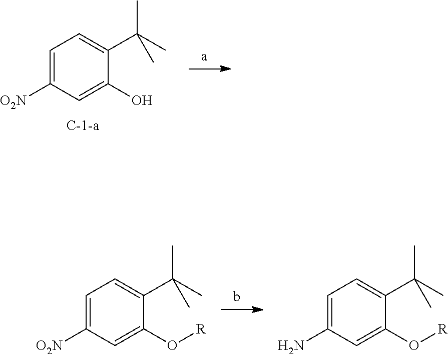

a) RX (X=Br, I), K₂CO₃ or Cs₂CO₃, DMF; b) HCO₂NH₄ or HCO₂K, Pd—C, EtOH

### Specific Example

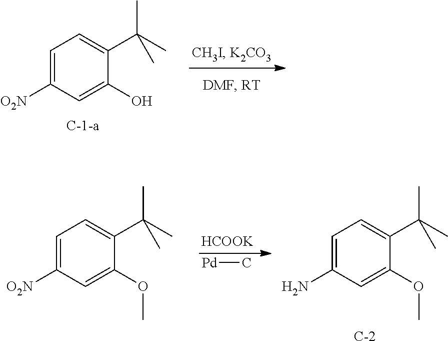

### 1-tert-Butyl-2-methoxy-4-nitrobenzene

To a mixture of 2-tert-butyl-5-nitrophenol (C-1-a) (100 mg, 0.52 mmol) and K₂CO₃ (86 mg, 0.62 mmol) in DMF (2 mL) was added CH₃I (40 μL, 0.62 mmol). The reaction mixture was stirred at room temperature for 2 h, diluted with water and extracted with EtOAc. The combined organic layers were washed with brine and dried over MgSO₄. After filtration, the filtrate was evaporated to dryness to give 1-tert-butyl-2-methoxy-4-nitrobenzene (82 mg, 76%) that was used without further purification. ¹H NMR (400 MHz, CDCl₃) δ 7.77 (t, J=4.3 Hz, 1H), 7.70 (d, J=2.3 Hz, 1H), 7.40 (d, J=8.6 Hz, 1H), 3.94 (s, 3H), 1.39 (s, 9H).

### C-2; 4-tert-Butyl-3-methoxyaniline

To a refluxing solution of 1-tert-butyl-2-methoxy-4-nitrobenzene (82 mg, 0.4 mmol) in EtOH (2 mL) was added potassium formate (300 mg, 3.6 mmol) in water (1 mL), followed by 10% Pd—C (15 mg). The reaction mixture was refluxed for additional 60 min, cooled to room temperature and filtered through Celite. The filtrate was concentrated to dryness to give 4-tert-butyl-3-methoxyaniline (C-2) (52 mg, 72%) that was used without further purification. HPLC ret. time 2.29 min, 10-99% CH₃CN, 5 min run; ESI-MS 180.0 m/z (MH⁺).

### Other Examples

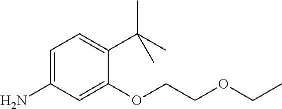 [SMILES: CCOCCOc1cc(N)ccc1C(C)(C)C]

### C-3; 3-(2-Ethoxyethoxy)-4-tert-butylbenzenamine

3-(2-Ethoxyethoxy)-4-tert-butylbenzenamine (C-3) was synthesized following the general scheme above starting from 2-tert-butyl-5-nitrophenol (C-1-a) and 1-bromo-2-ethoxyethane. ¹H NMR (400 MHz, CDCl₃) δ 6.97 (d, J=7.9 Hz, 1H), 6.17 (s, 1H), 6.14 (d, J=2.3 Hz, 1H), 4.00 (t, J=5.2 Hz, 2H), 3.76 (t, J=5.2 Hz, 2H), 3.53 (q, J=7.0 Hz, 2H), 1.27 (s, 9H), 1.16 (t, J=7.0 Hz, 3H); HPLC ret. time 2.55 min, 10-99% CH₃CN, 5 min run; ESI-MS 238.3 m/z (MH⁺).

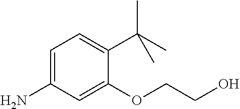

### C-4; 2-(2-tert-Butyl-5-aminophenoxy)ethanol

2-(2-tert-Butyl-5-aminophenoxy)ethanol (C-4) was synthesized following the general scheme above starting from 2-tert-butyl-5-nitrophenol (C-1-a) and 2-bromoethanol. HPLC ret. time 2.08 min, 10-99% CH₃CN, 5 min nm; ESI-MS 210.3 m/z (MH⁺).

### Example 3

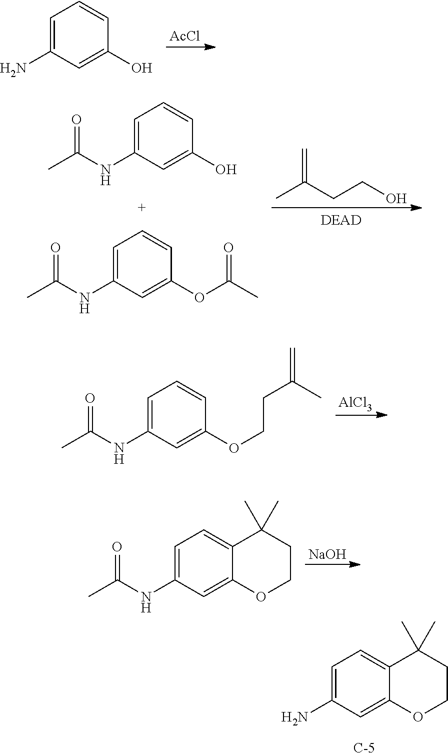

### N-(3-Hydroxy-phenyl)-acetamide and acetic acid 3-formylamino-phenyl ester

To a well stirred suspension of 3-amino-phenol (50 g, 0.46 mol) and NaHCO₃ (193.2 g, 2.3 mol) in chloroform (1 L) was added dropwise chloroacetyl chloride (46.9 g, 0.6 mol) over a period of 30 min at 0° C. After the addition was complete, the reaction mixture was refluxed overnight and then cooled to room temperature. The excess NaHCO₃ was removed via filtration. The filtrate was poured into water and extracted with EtOAc (300×3 mL). The combined organic layers were washed with brine (500 mL), dried over anhydrous Na₂SO₄ and concentrated under reduced pressure to give a mixture of N-(3-hydroxy-phenyl)-acetamide and acetic acid 3-formylamino-phenyl ester (35 g, 4:1 by NMR analysis). The mixture was used directly in the next step.

### N-[3-(3-Methyl-but-3-enyloxy)-phenyl]-acetamide

A suspension of the mixture of N-(3-hydroxy-phenyl)-acetamide and acetic acid 3-formylamino-phenyl ester (18.12 g, 0.12 mol), 3-methyl-but-3-en-1-ol (8.6 g, 0.1 mol), DEAD (87 g, 0.2 mol) and Ph₃P (31.44 g, 0.12 mol) in benzene (250 mL) was heated at reflux overnight and then cooled to room temperature. The reaction mixture was poured into water and the organic layer was separated. The aqueous phase was extracted with EtOAc (300×3 mL). The combined organic layers were washed with brine, dried over anhydrous Na₂SO₄ and concentrated. The residue was purified by column chromatography to give N-[3-(3-methyl-but-3-enyloxy)-phenyl]acetamide (11 g, 52%).

### N-(4,4-Dimethyl-chroman-7-yl)-acetamide

A mixture of N-[3-(3-methyl-but-3-enyloxy)-phenyl]-acetamide (2.5 g, 11.4 mmol) and AlCl₃ (4.52 g, 34.3 mmol) in fluoro-benzene (50 mL) was heated at reflux overnight. After cooling, the reaction mixture was poured into water. The organic layer was separated and the aqueous phase was extracted with EtOAc (40×3 mL). The combined organic layers were washed with brine, dried over anhydrous Na₂SO₄ and concentrated under vacuum. The residue was purified by column chromatography to give N-(4,4-dimethyl-chroman-7-yl)-acetamide (1.35 g, 54%).

### C-5; 3,4-Dihydro-4,4-dimethyl-2H-chromen-7-amine

A mixture of N-(4,4-dimethyl-chroman-7-yl)-acetamide (1.35 g, 6.2 mmol) in 20% HCl solution (30 mL) was heated at reflux for 3 h and then cooled to room temperature. The reaction mixture was basified with 10% aq. NaOH to pH 8 and extracted with EtOAc (30×3 mL). The combined organic layers were washed with brine, dried over anhydrous Na₂SO₄ and concentrated to give 3,4-dihydro-4,4-dimethyl-2H-chromen-7-amine (C-5) (1 g, 92%). ¹H NMR (DMSO-d₆) δ 6.87 (d, J=8.4 Hz, 1H), 6.07 (dd, J=8.4, 2.4 Hz, 1H), 5.87 (d, J=2.4 Hz, 1H), 4.75 (s, 2H), 3.99 (t, J=5.4 Hz, 2H), 1.64 (t, J=5.1 Hz, 2H), 1.15 (s, 6H); ESI-MS 178.1 m/z (MH⁺).

### Example 4

### General Scheme

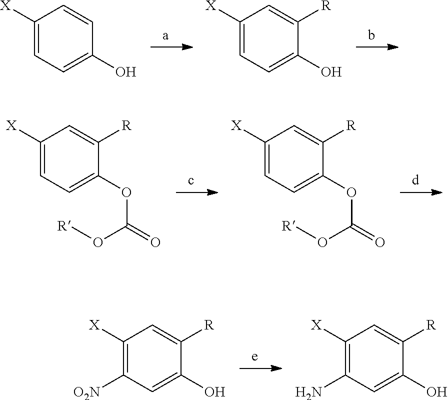

X═F, Cl; a) ROH, H₂SO₄ or MeSO₃H, CH₂Cl₂; b) R′CO₂Cl, Et₃N, 1,4-dioxane or CHCl₃; c) HNO₃, H₂SO₄ or KNO₃, H₂SO₄ or HNO₃, AcOH; d) piperidine, CH₂Cl₂; e) HCO₂NH₄, Pd—C, EtOH or SnCl₂.2H₂O, EtOH or H₂, Pd—C, MeOH.

### Specific Example

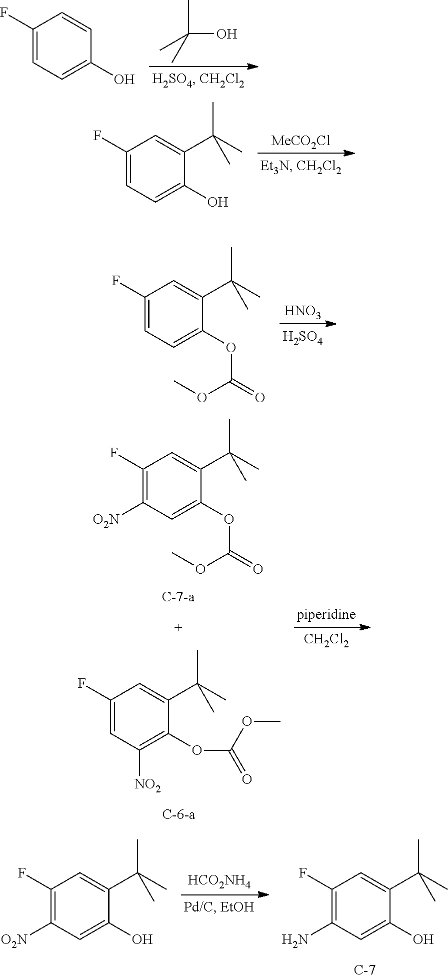

### 2-tert-Butyl-4-fluorophenol

4-Fluorophenol (5 g, 45 mmol) and tert-butanol (5.9 mL, 63 mmol) were dissolved in CH₂Cl₂ (80 mL) and treated with concentrated sulfuric acid (98%, 3 mL). The mixture was stirred at room temperature overnight. The organic layer was washed with water, neutralized with NaHCO₃, dried over MgSO₄ and concentrated. The residue was purified by column chromatography (5-15% EtOAc-Hexane) to give 2-tert-butyl-4-fluorophenol (3.12 g, 42%). ¹H NMR (400 MHz, DMSO-d₆) δ 9.32 (s, 1H), 6.89 (dd, J=11.1, 3.1 Hz, 1H), 6.84-6.79 (m, 1H), 6.74 (dd, J=8.7, 5.3 Hz, 1H), 1.33 (s, 9H).

### 2-tert-Butyl-4-fluorophenyl methyl carbonate

To a solution of 2-tert-butyl-4-fluorophenol (2.63 g, 15.7 mmol) and NEt₃ (3.13 mL, 22.5 mmol) in dioxane (45 mL) was added methyl chloroformate (1.27 mL, 16.5 mmol). The mixture was stirred at room temperature for 1 h. The precipitate was removed via filtration. The filtrate was then diluted with water and extracted with ether. The ether extract was washed with water and dried over MgSO₄. After removal of solvent, the residue was purified by column chromatography to give 2-tert-butyl-4-fluorophenyl methyl carbonate (2.08 g, 59%). ¹H NMR (400 MHz, DMSO-d₆) δ 7.24 (dd, J=8.8, 5.4 Hz, 1H), 7.17-7.10 (m, 2H), 3.86 (s, 3H), 1.29 (s, 9H).

### 2-tert-Butyl-4-fluoro-5-nitrophenyl methyl carbonate (C-7-a) and 2-tert-butyl-4-fluoro-6-nitrophenyl methyl carbonate (C-6-a)

To a solution of 2-tert-butyl-4-fluorophenyl methyl carbonate (1.81 g, 8 mmol) in H₂SO₄ (98%, 1 mL) was added slowly a cooled mixture of H₂SO₄ (1 mL) and HNO₃ (1 mL) at 0° C. The mixture was stirred for 2 h while warming to room temperature, poured into ice and extracted with diethyl ether. The ether extract was washed with brine, dried over MgSO₄ and concentrated. The residue was purified by column chromatography (0-10% EtOAc-Hexane) to give 2-tert-butyl-4-fluoro-5-nitrophenyl methyl carbonate (C-7-a) (1.2 g, 55%) and 2-tert-butyl-4-fluoro-6-nitrophenyl methyl carbonate (C-6-a) (270 mg, 12%). 2-tert-Butyl-4-fluoro-5-nitrophenyl methyl carbonate (C-7-a): ¹H NMR (400 MHz, DMSO-d₆) δ 8.24 (d, J=7.1 Hz, 1H), 7.55 (d, J=13.4 Hz, 1H), 3.90 (s, 3H), 1.32 (s, 9H). 2-tert-butyl-4-fluoro-6-nitrophenyl methyl carbonate (C-6-a): ¹H NMR (400 MHz, DMSO-d₆) δ 8.04 (dd, J=7.6, 3.1 Hz, 1H), 7.69 (dd, J=10.1, 3.1 Hz, 1H), 3.91 (s, 3H), 1.35 (s, 9H).

### 2-tert-Butyl-4-fluoro-5-nitrophenol

To a solution of 2-tert-butyl-4-fluoro-5-nitrophenyl methyl carbonate (C-7-a) (1.08 g, 4 mmol) in CH₂Cl₂ (40 mL) was added piperidine (3.94 mL, 10 mmol). The mixture was stirred at room temperature for 1 h and extracted with 1N NaOH (3×). The aqueous layer was acidified with 1N HCl and extracted with diethyl ether. The ether extract was washed with brine, dried (MgSO₄) and concentrated to give 2-tert-butyl-4-fluoro-5-nitrophenol (530 mg, 62%). ¹H NMR (400 MHz, DMSO-d₆) δ 10.40 (s, 1H), 7.49 (d, J=6.8 Hz, 1H), 7.25 (d, J=13.7 Hz, 1H), 1.36 (s, 9H).

### C-7; 2-tert-Butyl-5-amino-4-fluorophenol

To a refluxing solution of 2-tert-butyl-4-fluoro-5-nitrophenol (400 mg, 1.88 mmol) and ammonium formate (400 mg, 6.1 mmol) in EtOH (20 mL) was added 5% Pd—C (260 mg). The mixture was refluxed for additional 1 h, cooled and filtered through Celite. The solvent was removed by evaporation to give 2-tert-butyl-5-amino-4-fluorophenol (C-7) (550 mg, 83%). ¹H NMR (400 MHz, DMSO-d₆) δ 8.83 (br s, 1H), 6.66 (d, J=13.7 Hz, 1H), 6.22 (d, J=8.5 Hz, 1H), 4.74 (br s, 2H), 1.26 (s, 9H); HPLC ret. time 2.58 min, 10-99% CH₃CN, 5 min run; ESI-MS 184.0 m/z (MH⁺).

### Other Examples

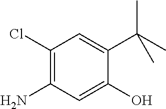

### C-10; 2-tert-Butyl-5-amino-4-chlorophenol

2-tert-Butyl-5-amino-4-chlorophenol (C-10) was synthesized following the general scheme above starting from 4-chlorophenol and tert-butanol. Overall yield (6%). HPLC ret. time 3.07 min, 10-99% CH₃CN, 5 min run; ESI-MS 200.2 m/z (MH⁺).

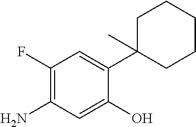 [SMILES: CC1(c2cc(F)c(N)cc2O)CCCCC1]

### C-13; 5-Amino-4-fluoro-2-(1-methylcyclohexyl)phenol

5-Amino-4-fluoro-2-(1-methylcyclohexyl)phenol (C-13) was synthesized following the general scheme above starting from 4-fluorophenol and 1-methylcyclohexanol. Overall yield (3%). HPLC ret. time 3.00 min, 10-99% CH₃CN, 5 min run; ESI-MS 224.2 m/z (MH⁺).

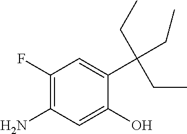 [SMILES: CCC(CC)(CC)c1cc(F)c(N)cc1O]

### C-19; 5-Amino-2-(3-ethylpentan-3-yl)-4-fluoro-phenol

5-Amino-2-(3-ethylpentan-3-yl)-4-fluoro-phenol (C-19) was synthesized following the general scheme above starting from 4-fluorophenol and 3-ethyl-3-pentanol. Overall yield (1%).

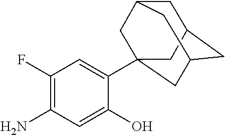 [SMILES: Nc1cc(O)c(C23CC4CC(CC(C4)C2)C3)cc1F]

### C-20; 2-Admantyl-5-amino-4-fluoro-phenol

2-Admantyl-5-amino-4-fluoro-phenol (C-20) was synthesized following the general scheme above starting from 4-fluorophenol and adamantan-1-ol.

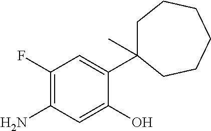 [SMILES: CC1(c2cc(F)c(N)cc2O)CCCCCC1]

### C-21; 5-Amino-4-fluoro-2-(1-methylcycloheptyl)phenol

5-Amino-4-fluoro-2-(1-methylcycloheptyl)phenol (C-21) was synthesized following the general scheme above starting from 4-fluorophenol and 1-methyl-cycloheptanol.

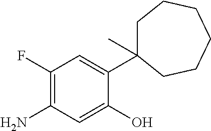 [SMILES: CC1(c2cc(F)c(N)cc2O)CCCCCC1]

### C-22; 5-Amino-4-fluoro-2-(1-methylcyclooctyl)phenol

5-Amino-4-fluoro-2-(1-methylcyclooctyl)phenol (C-22) was synthesized following the general scheme above starting from 4-fluorophenol and 1-methyl-cyclooctanol.

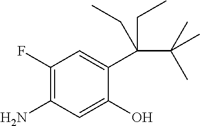 [SMILES: CCC(CC)(c1cc(F)c(N)cc1O)C(C)(C)C]

### C-23; 5-Amino-2-(3-ethyl-2,2-dimethylpentan-3-yl)-4-fluoro-phenol

5-Amino-2-(3-ethyl-2,2-dimethylpentan-3-yl)-4-fluoro-phenol (C-23) was synthesized following the general scheme above starting from 4-fluorophenol and 3-ethyl-2,2-dimethyl-pentan-3-ol.

### Example 5

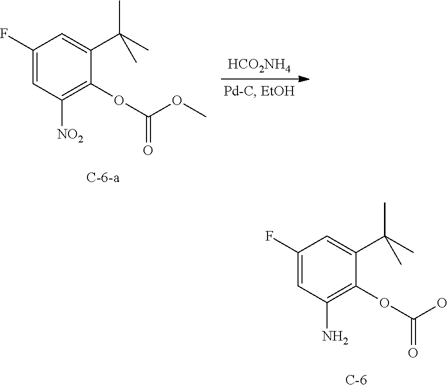

### C-6; 2-tert-Butyl-4-fluoro-6-aminophenyl methyl carbonate

To a refluxing solution of 2-tert-butyl-4-fluoro-6-nitrophenyl methyl carbonate (250 mg, 0.92 mmol) and ammonium formate (250 mg, 4 mmol) in EtOH (10 mL) was added 5% Pd—C (170 mg). The mixture was refluxed for additional 1 h, cooled and filtered through Celite. The solvent was removed by evaporation and the residue was purified by column chromatography (0-15%, EtOAc-Hexane) to give 2-tert-butyl-4-fluoro-6-aminophenyl methyl carbonate (C-6) (60 mg, 27%). HPLC ret. time 3.35 min, 10-99% CH₃CN, 5 min run; ESI-MS 242.0 m/z (MIT).

### Example 6

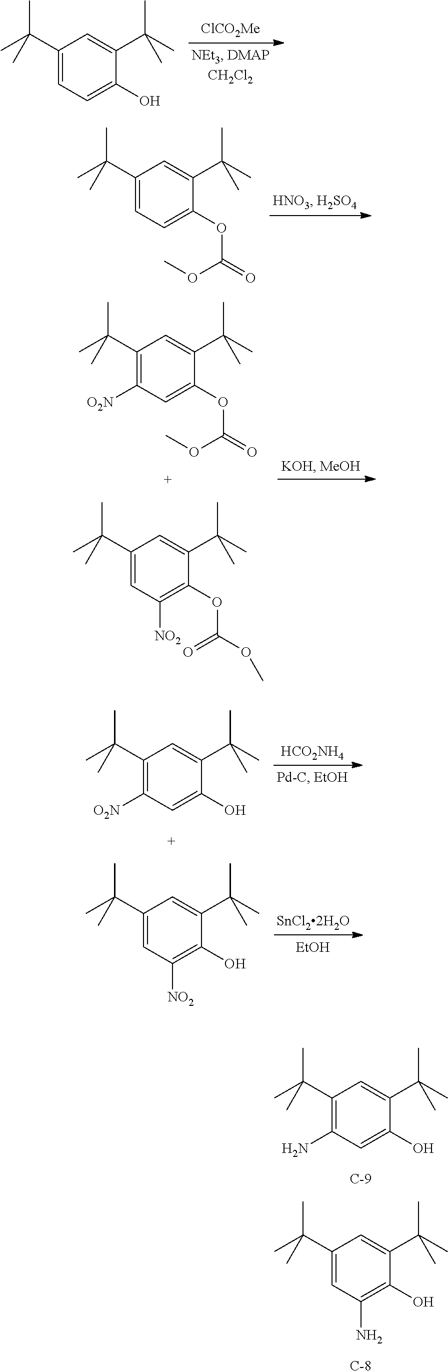

### Carbonic acid 2,4-di-tert-butyl-phenyl ester methyl ester

Methyl chloroformate (58 mL, 750 mmol) was added dropwise to a solution of 2,4-di-tert-butyl-phenol (103.2 g, 500 mmol), Et₃N (139 mL, 1000 mmol) and DMAP (3.05 g, 25 mmol) in dichloromethane (400 mL) cooled in an ice-water bath to 0° C. The mixture was allowed to warm to room temperature while stirring overnight, then filtered through silica gel (approx. 1 L) using 10% ethyl acetate-hexanes (˜4 L) as the eluent. The combined filtrates were concentrated to yield carbonic acid 2,4-di-tert-butyl-phenyl ester methyl ester as a yellow oil (132 g, quant.). ¹H NMR (400 MHz, DMSO-d₆) δ 7.35 (d, J=2.4 Hz, 1H), 7.29 (dd, J=8.5, 2.4 Hz, 1H), 7.06 (d, J=8.4 Hz, 1H), 3.85 (s, 3H), 1.30 (s, 9H), 1.29 (s, 9H).

### Carbonic acid 2,4-di-tert-butyl-5-nitro-phenyl ester methyl ester and Carbonic acid 2,4-di-tert-butyl-6-nitro-phenyl ester methyl ester

To a stirring mixture of carbonic acid 2,4-di-tert-butyl-phenyl ester methyl ester (4.76 g, 18 mmol) in conc. sulfuric acid (2 mL), cooled in an ice-water bath, was added a cooled mixture of sulfuric acid (2 mL) and nitric acid (2 mL). The addition was done slowly so that the reaction temperature did not exceed 50° C. The reaction was allowed to stir for 2 h while warming to room temperature. The reaction mixture was then added to ice-water and extracted into diethyl ether. The ether layer was dried (MgSO₄), concentrated and purified by column chromatography (0-10% ethyl acetate-hexanes) to yield a mixture of carbonic acid 2,4-di-tert-butyl-5-nitro-phenyl ester methyl ester and carbonic acid 2,4-di-tert-butyl-6-nitro-phenyl ester methyl ester as a pale yellow solid (4.28 g), which was used directly in the next step.

### 2,4-Di-tert-butyl-5-nitro-phenol and 2,4-Di-tert-butyl-6-nitro-phenol

The mixture of carbonic acid 2,4-di-tert-butyl-5-nitro-phenyl ester methyl ester and carbonic acid 2,4-di-tert-butyl-6-nitro-phenyl ester methyl ester (4.2 g, 12.9 mmol) was dissolved in MeOH (65 mL) and KOH (2.0 g, 36 mmol) was added. The mixture was stirred at room temperature for 2 h. The reaction mixture was then made acidic (pH 2-3) by adding conc. HCl and partitioned between water and diethyl ether. The ether layer was dried (MgSO₄), concentrated and purified by column chromatography (0-5% ethyl acetate-hexanes) to provide 2,4-di-tert-butyl-5-nitro-phenol (1.31 g, 29% over 2 steps) and 2,4-di-tert-butyl-6-nitro-phenol. 2,4-Di-tert-butyl-5-nitro-phenol: ¹H NMR (400 MHz, DMSO-d₆) δ 10.14 (s, 1H, OH), 7.34 (s, 1H), 6.83 (s, 1H), 1.36 (s, 9H), 1.30 (s, 9H). 2,4-Di-tert-butyl-6-nitro-phenol: ¹H NMR (400 MHz, CDCl₃) δ 11.48 (s, 1H), 7.98 (d, J=2.5 Hz, 1H), 7.66 (d, J=2.4 Hz, 1H), 1.47 (s, 9H), 1.34 (s, 9H).

### C-9; 5-Amino-2,4-di-tert-butyl-phenol

To a refluxing solution of 2,4-di-tert-butyl-5-nitro-phenol (1.86 g, 7.4 mmol) and ammonium formate (1.86 g) in ethanol (75 mL) was added Pd-5% wt. on activated carbon (900 mg). The reaction mixture was stirred at reflux for 2 h, cooled to room temperature and filtered through Celite. The Celite was washed with methanol and the combined filtrates were concentrated to yield 5-amino-2,4-di-tert-butyl-phenol as a grey solid (1.66 g, quant.). ¹H NMR (400 MHz, DMSO-d₆) δ 8.64 (s, 1H, OH), 6.84 (s, 1H), 6.08 (s, 1H), 4.39 (s, 2H, NH₂), 1.27 (m, 18H); HPLC ret. time 2.72 min, 10-99% CH₃CN, 5 min run; ESI-MS 222.4 m/z (MH⁺).

### C-8; 6-Amino-2,4-di-tert-butyl-phenol

A solution of 2,4-di-tert-butyl-6-nitro-phenol (27 mg, 0.11 mmol) and SnCl₂.2H₂O (121 mg, 0.54 mmol) in EtOH (1.0 mL) was heated in microwave oven at 100° C. for 30 min. The mixture was diluted with EtOAc and water, basified with sat. NaHCO₃ and filtered through Celite. The organic layer was separated and dried over Na₂SO₄. Solvent was removed by evaporation to provide 6-amino-2,4-di-tert-butyl-phenol (C-8), which was used without further purification. HPLC ret. time 2.74 min, 10-99% CH₃CN, 5 min run; ESI-MS 222.5 m/z (MH⁺).

### Example 7

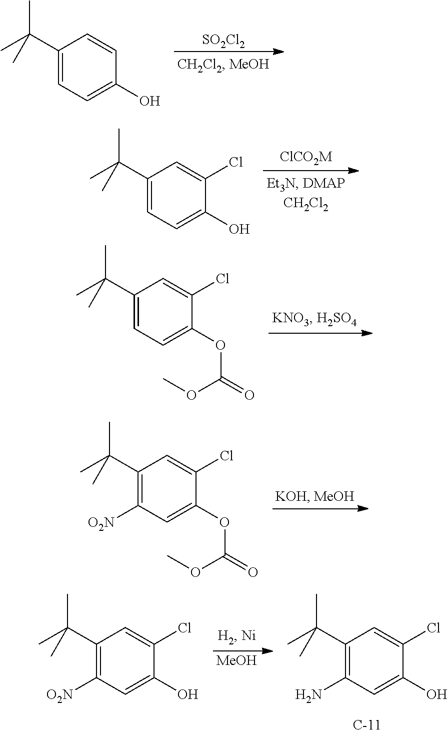

### 4-tert-butyl-2-chloro-phenol

To a solution of 4-tert-butyl-phenol (40.0 g, 0.27 mol) and SO₂Cl₂ (37.5 g, 0.28 mol) in CH₂Cl₂ was added MeOH (9.0 g, 0.28 mol) at 0° C. After addition was complete, the mixture was stirred overnight at room temperature and then water (200 mL) was added. The resulting solution was extracted with ethyl acetate. The combined organic layers were dried over anhydrous Na₂SO₄, filtered and concentrated under vacuum. The residue was purified by column chromatography (Pet. Ether/EtOAc, 50:1) to give 4-tert-butyl-2-chloro-phenol (47.0 g, 95%).

### 4-tert-Butyl-2-chlorophenyl methyl carbonate

To a solution of 4-tert-butyl-2-chlorophenol (47.0 g, 0.25 mol) in dichloromethane (200 mL) was added Et₃N (50.5 g, 0.50 mol), DMAP (1 g) and methyl chloroformate (35.4 g, 0.38 mol) at 0° C. The reaction was allowed to warm to room temperature and stirred for additional 30 min. The reaction mixture was washed with H₂O and the organic layer was dried over Na₂SO₄ and concentrated to give 4-tert-butyl-2-chlorophenyl methyl carbonate (56.6 g, 92%), which was used directly in the next step.

### 4-tert-Butyl-2-chloro-5-nitrophenyl methyl carbonate

4-tert-Butyl-2-chlorophenyl methyl carbonate (36.0 g, 0.15 mol) was dissolved in conc. H₂SO₄ (100 mL) at 0° C. KNO₃ (0.53 g, 5.2 mmol) was added in portions over 25 min. The reaction was stirred for 1.5 h and poured into ice (200 g). The aqueous layer was extracted with dichloromethane. The combined organic layers were washed with aq. NaHCO₃, dried over Na₂SO₄ and concentrated under vacuum to give 4-tert-butyl-2-chloro-5-nitrophenyl methyl carbonate (41.0 g), which was used without further purification.

### 4-tert-Butyl-2-chloro-5-nitro-phenol

Potassium hydroxide (10.1 g, 181 mmol) was added to 4-tert-butyl-2-chloro-5-nitrophenyl methyl carbonate (40.0 g, 139 mmol) in MeOH (100 mL). After 30 min, the reaction was acidified with 1N HCl and extracted with dichloromethane. The combined organic layers were combined, dried over Na₂SO₄ and concentrated under vacuum. The crude residue was purified by column chromatography (Pet. Ether/EtOAc, 30:1) to give 4-tert-butyl-2-chloro-5-nitro-phenol (23.0 g, 68% over 2 steps).

### C-11; 4-tert-Butyl-2-chloro-5-amino-phenol

To a solution of 4-tert-butyl-2-chloro-5-nitro-phenol (12.6 g, 54.9 mmol) in MeOH (50 mL) was added Ni (1.2 g). The reaction was shaken under H₂ (1 atm) for 4 h. The reaction mixture was filtered and the filtrate was concentrated. The residue was purified by column chromatography (P.E./EtOAc, 20:1) to give 4-tert-butyl-2-chloro-5-amino-phenol (C-11) (8.5 g, 78%). ¹H NMR (DMSO-d₆) δ 9.33 (s, 1H), 6.80 (s, 1H), 6.22 (s, 1H), 4.76 (s, 1H), 1.23 (s, 9H); ESI-MS 200.1 m/z (MH⁺).

### Example 8

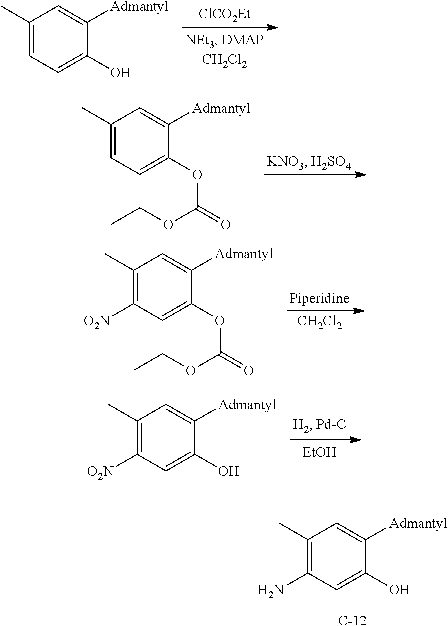

### 2-Admantyl-4-methyl-phenyl ethyl carbonate

Ethyl chloroformate (0.64 mL, 6.7 mmol) was added dropwise to a solution of 2-admantyl-4-methylphenol (1.09 g, 4.5 mmol), Et₃N (1.25 mL, 9 mmol) and DMAP (catalytic amount) in dichloromethane (8 mL) cooled in an ice-water bath to 0° C. The mixture was allowed to warm to room temperature while stirring overnight, then filtered and the filtrate was concentrated. The residue was purified by column chromatography (10-20% ethyl acetate-hexanes) to yield 2-admantyl-4-methyl-phenyl ethyl carbonate as a yellow oil (1.32 g, 94%).

### 2-Admantyl-4-methyl-5-nitrophenyl ethyl carbonate

To a cooled solution of 2-admantyl-4-methyl-phenyl ethyl carbonate (1.32 g, 4.2 mmol) in H₂SO₄ (98%, 10 mL) was added KNO₃ (510 mg, 5.0 mmol) in small portions at 0° C. The mixture was stirred for 3 h while warming to room temperature, poured into ice and then extracted with dichloromethane. The combined organic layers were washed with NaHCO₃ and brine, dried over MgSO₄ and concentrated to dryness. The residue was purified by column chromatography (0-10% EtOAc-Hexane) to yield 2-admantyl-4-methyl-5-nitrophenyl ethyl carbonate (378 mg, 25%).

### 2-Admantyl-4-methyl-5-nitrophenol

To a solution of 2-admantyl-4-methyl-5-nitrophenyl ethyl carbonate (378 mg, 1.05 mmol) in CH₂Cl₂ (5 mL) was added piperidine (1.0 mL). The solution was stirred at room temperature for 1 h, adsorbed onto silica gel under reduced pressure and purified by flash chromatography on silica gel (0-15%, EtOAc-Hexanes) to provide 2-admantyl-4-methyl-5-nitrophenol (231 mg, 77%).

### C-12; 2-Admantyl-4-methyl-5-aminophenol

To a solution of 2-admantyl-4-methyl-5-nitrophenol (231 mg, 1.6 mmol) in EtOH (2 mL) was added Pd-5% wt on carbon (10 mg). The mixture was stirred under H₂ (1 atm) overnight and then filtered through Celite. The filtrate was evaporated to dryness to provide 2-admantyl-4-methyl-5-aminophenol (C-12), which was used without further purification. HPLC ret. time 2.52 min, 10-99% CH₃CN, 5 min run; ESI-MS 258.3 m/z (MH⁺).

### Example 9

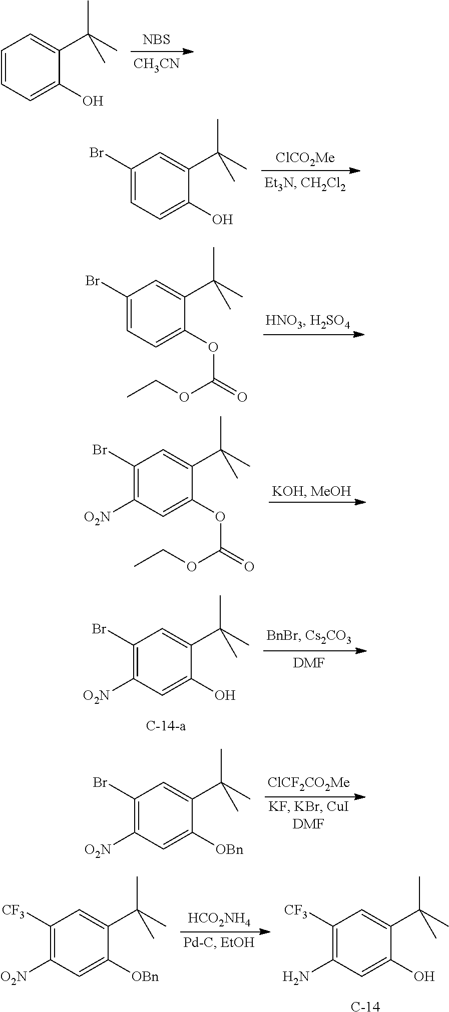

### 2-tert-Butyl-4-bromophenol

To a solution of 2-tert-butylphenol (250 g, 1.67 mol) in CH₃CN (1500 mL) was added NBS (300 g, 1.67 mol) at room temperature. After addition, the mixture was stirred at room temperature overnight and then the solvent was removed. Petroleum ether (1000 mL) was added, and the resulting white precipitate was filtered off. The filtrate was concentrated under reduced pressure to give the crude 2-tert-butyl-4-bromophenol (380 g), which was used without further purification.

### Methyl(2-tert-butyl-4-bromophenyl)carbonate

To a solution of 2-t-butyl-4-bromophenol (380 g, 1.67 mol) in dichloromethane (1000 mL) was added Et₃N (202 g, 2 mol) at room temperature. Methyl chloroformate (155 mL) was added dropwise to the above solution at 0° C. After addition, the mixture was stirred at 0° C. for 2 h., quenched with saturated ammonium chloride solution and diluted with water. The organic layer was separated and washed with water and brine, dried over Na₂SO₄, and concentrated to provide the crude methyl (2-tert-butyl-4-bromophenyl)carbonate (470 g), which was used without further purification.

### Methyl(2-tert-butyl-4-bromo-5-nitrophenyl)carbonate

Methyl(2-tert-butyl-4-bromophenyl)carbonate (470 g, 1.67 mol) was dissolved in conc. H₂SO₄ (1000 ml) at 0° C. KNO₃ (253 g, 2.5 mol) was added in portions over 90 min. The reaction mixture was stirred at 0° C. for 2 h and poured into ice-water (20 L). The resulting precipitate was collected via filtration and washed with water thoroughly, dried and recrystallized from ether to give methyl (2-tert-butyl-4-bromo-5-nitrophenyl) carbonate (332 g, 60% over 3 steps).

### C-14-a; 2-tert-Butyl-4-bromo-5-nitro-phenol

To a solution of methyl (2-tert-butyl-4-bromo-5-nitrophenyl)carbonate (121.5 g, 0.366 mol) in methanol (1000 mL) was added potassium hydroxide (30.75 g, 0.549 mol) in portions. After addition, the mixture was stirred at room temperature for 3 h and acidified with 1N HCl to pH 7. Methanol was removed and water was added. The mixture was extracted with ethyl acetate and the organic layer was separated, dried over Na₂SO₄ and concentrated to give 2-tert-butyl-4-bromo-5-nitro-phenol (C-14-a) (100 g, 99%).

### 1-tert-Butyl-2-(benzyloxy)-5-bromo-4-nitrobenzene

To a mixture of 2-tert-butyl-4-bromo-5-nitrophenol (C-14-a) (1.1 g, 4 mmol) and Cs₂CO₃ (1.56 g, 4.8 mmol) in DMF (8 mL) was added benzyl bromide (500 μL, 4.2 mmol). The mixture was stirred at room temperature for 4 h, diluted with H₂O and extracted twice with EtOAc. The combined organic layers were washed with brine and dried over MgSO₄. After removal of solvent, the residue was purified by column chloromatography (0-5% EtOAc-Hexane) to yield 1-tert-butyl-2-(benzyloxy)-5-bromo-4-nitrobenzene (1.37 g, 94%). ¹H NMR (400 MHz, CDCl₃) 7.62 (s, 1H), 7.53 (s, 1H), 7.43 (m, 5H), 5.22 (s, 2H), 1.42 (s, 9H).

### 1-tert-Butyl-2-(benzyloxy)-5-(trifluoromethyl)-4-nitrobenzene

A mixture of 1-tert-butyl-2-(benzyloxy)-5-bromo-4-nitrobenzene (913 mg, 2.5 mmol), KF (291 mg, 5 mmol), KBr (595 mg, 5 mmol), CuI (570 mg, 3 mmol), methyl chlorodifluoroacetate (1.6 mL, 15 mmol) and DMF (5 mL) was stirred at 125° C. in a sealed tube overnight, cooled to room temperature, diluted with water and extracted three times with EtOAc. The combined organic layers were washed with brine and dried over anhydrous MgSO₄. After removal of the solvent, the residue was purified by column chromatography (0-5% EtOAc-Hexane) to yield 1-tert-butyl-2-(benzyloxy)-5-(trifluoromethyl)-4-nitrobenzene (591 mg, 67%). ¹H NMR (400 MHz, CDCl₃) 7.66 (s, 1H), 7.37 (m, 5H), 7.19 (s, 1H), 5.21 (s, 2H), 1.32 (s, 9H).

### C-14; 5-Amino-2-tert-butyl-4-trifluoromethyl-phenol

To a refluxing solution of 1-tert-butyl-2-(benzyloxy)-5-(trifluoromethyl)-4-nitrobenzene (353 mg, 1.0 mmol) and ammonium formate (350 mg, 5.4 mmol) in EtOH (10 mL) was added 10% Pd—C (245 mg). The mixture was refluxed for additional 2 h, cooled to room temperature and filtered through Celite. After removal of solvent, the residue was purified by column chromatography to give 5-Amino-2-tert-butyl-4-trifluoromethyl-phenol (C-14) (120 mg, 52%). ¹H NMR (400 MHz, CDCl₃) δ 7.21 (s, 1H), 6.05 (s, 1H), 1.28 (s, 9H); HPLC ret. time 3.46 min, 10-99% CH₃CN, 5 min run; ESI-MS 234.1 m/z (MH⁺).

### Example 10

### General Scheme

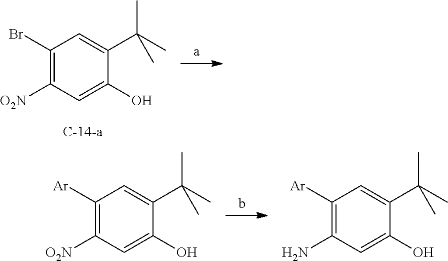

a) ArB(OH)₂, K₂CO₃, Pd(PPh₃)₄, H₂O, DMF or ArB(OH)₂, (dppf)PdCl₂, K₂CO₃, EtOH; b) H₂, Raney Ni, MeOH or HCO₂NH₄, Pd—C, EtOH or SnCl₂.2H₂O.

### Specific Example

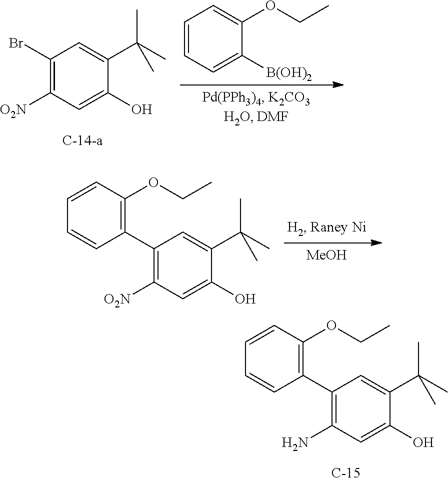

### 2-tert-Butyl-4-(2-ethoxyphenyl)-5-nitrophenol

To a solution of 2-tert-butyl-4-bromo-5-nitrophenol (C-14-a) (8.22 g, 30 mmol) in DMF (90 mL) was added 2-ethoxyphenyl boronic acid (5.48 g, 33 mmol), potassium carbonate (4.56 g, 33 mmol), water (10 ml) and Pd(PPh₃)₄ (1.73 g, 1.5 mmol). The mixture was heated at 90° C. for 3 h under nitrogen. The solvent was removed under reduced pressure. The residue was partitioned between water and ethyl acetate. The combined organic layers were washed with water and brine, dried and purified by column chromatography (petroleum ether-ethyl acetate, 10:1) to afford 2-tert-butyl-4-(2-ethoxyphenyl)-5-nitrophenol (9.2 g, 92%). ¹HNMR (DMSO-d₆) δ 10.38 (s, 1H), 7.36 (s, 1H), 7.28 (m, 2H), 7.08 (s, 1H), 6.99 (t, 1H, J=7.35 Hz), 6.92 (d, 1H, J=8.1 Hz), 3.84 (q, 2H, J=6.6 Hz), 1.35 (s, 9H), 1.09 (t, 3H, J=6.6 Hz); ESI-MS 314.3 m/z (MH⁺).

### C-15; 2-tert-Butyl-4-(2-ethoxyphenyl)-5-aminophenol

To a solution of 2-tert-butyl-4-(2-ethoxyphenyl)-5-nitrophenol (3.0 g, 9.5 mmol) in methanol (30 ml) was added Raney Ni (300 mg). The mixture was stirred under H₂ (1 atm) at room temperature for 2 h. The catalyst was filtered off and the filtrate was concentrated. The residue was purified by column chromatography (petroleum ether-ethyl acetate, 6:1) to afford 2-tert-butyl-4-(2-ethoxyphenyl)-5-aminophenol (C-15) (2.35 g, 92%). ¹HNMR (DMSO-d₆) δ 8.89 (s, 1H), 7.19 (t, 1H, J=4.2 Hz), 7.10 (d, 1H, J=1.8 Hz), 7.08 (d, 1H, J=1.8 Hz), 6.94 (t, 1H, J=3.6 Hz), 6.67 (s, 1H), 6.16 (s, 1H), 4.25 (s, 1H), 4.00 (q, 2H, J=6.9 Hz), 1.26 (s, 9H), 1.21 (t, 3H, J=6.9 Hz); ESI-MS 286.0 m/z (MH⁺).

### Other Examples

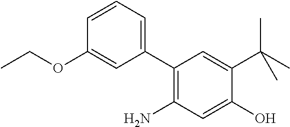 [SMILES: CCOc1cccc(-c2cc(C(C)(C)C)c(O)cc2N)c1]

### C-16; 2-tert-Butyl-4-(3-ethoxyphenyl)-5-aminophenol

2-tert-Butyl-4-(3-ethoxyphenyl)-5-aminophenol (C-16) was synthesized following the general scheme above starting from 2-tert-butyl-4-bromo-5-nitrophenol (C-14-a) and 3-ethoxyphenyl boronic acid. HPLC ret. time 2.77 min, 10-99% CH₃CN, 5 min run; ESI-MS 286.1 m/z (MH⁺).

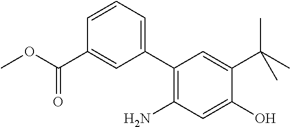 [SMILES: COC(=O)c1cccc(-c2cc(C(C)(C)C)c(O)cc2N)c1]

### C-17; 2-tert-Butyl-4-(3-methoxycarbonylphenyl)-5-aminophenol (C-17)

2-tert-Butyl-4-(3-methoxycarbonylphenyl)-5-aminophenol (C-17) was synthesized following the general scheme above starting from 2-tert-butyl-4-bromo-5-nitrophenol (C-14-a) and 3-(methoxycarbonyl)phenylboronic acid. HPLC ret. time 2.70 min, 10-99% CH₃CN, 5 min run; ESI-MS 300.5 m/z (MH⁺).

### Example 11

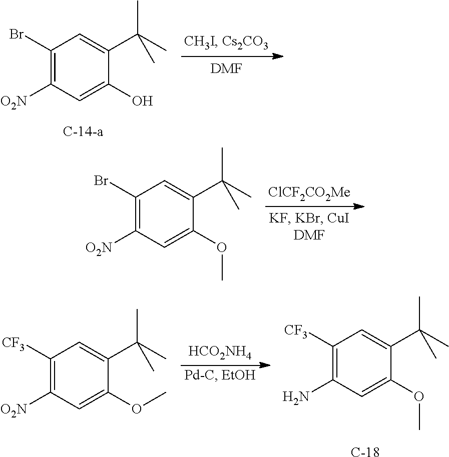

### 1-tert-Butyl-2-methoxy-5-bromo-4-nitrobenzene

To a mixture of 2-tert-butyl-4-bromo-5-nitrophenol (C-14-a) (1.5 g, 5.5 mmol) and Cs₂CO₃ (2.2 g, 6.6 mmol) in DMF (6 mL) was added methyl iodide (5150 μL, 8.3 mmol). The mixture was stirred at room temperature for 4 h, diluted with H₂O and extracted twice with EtOAc. The combined organic layers were washed with brine and dried over MgSO₄. After removal of solvent, the residue was washed with hexane to yield 1-tert-butyl-2-methoxy-5-bromo-4-nitrobenzene (1.1 g, 69%). ¹H NMR (400 MHz, CDCl₃) δ 7.58 (s, 1H), 7.44 (s, 1H), 3.92 (s, 3H), 1.39 (s, 9H).

### 1-tert-Butyl-2-methoxy-5-(trifluoromethyl)-4-nitrobenzene

A mixture of 1-tert-butyl-2-methoxy-5-bromo-4-nitrobenzene (867 mg, 3.0 mmol), KF (348 mg, 6 mmol), KBr (714 mg, 6 mmol), CuI (684 mg, 3.6 mmol), methyl chlorodifluoroacetate (2.2 mL, 21.0 mmol) in DMF (5 mL) was stirred at 125° C. in a sealed tube overnight, cooled to room temperature, diluted with water and extracted three times with EtOAc. The combined organic layers were washed with brine and dried over anhydrous MgSO₄. After removal of the solvent, the residue was purified by column chromatography (0-5% EtOAc-Hexane) to yield 1-tert-butyl-2-methoxy-5-(trifluoromethyl)-4-nitrobenzene (512 mg, 61%). ¹H NMR (400 MHz, CDCl₃) δ 7.60 (s, 1H), 7.29 (s, 1H), 3.90 (s, 3H), 1.33 (s, 9H).

### C-18; 1-tert-Butyl-2-methoxy-5-(trifluoromethyl)-4-aminobenzene

To a refluxing solution of 1-tert-butyl-2-methoxy-5-(trifluoromethyl)-4-nitrobenzene (473 mg, 1.7 mmol) and ammonium formate (473 mg, 7.3 mmol) in EtOH (10 mL) was added 10% Pd—C (200 mg). The mixture was refluxed for 1 h, cooled and filtered through Celite. The solvent was removed by evaporation to give 1-tert-butyl-2-methoxy-5-(trifluoromethyl)-4-aminobenzene (C-18) (403 mg, 95%). ¹H NMR (400 MHz, CDCl₃) δ 7.19 (s, 1H), 6.14 (s, 1H), 4.02 (bs, 2H), 3.74 (s, 3H), 1.24 (s, 9H).

### Example 12

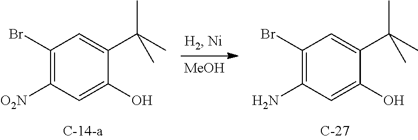

### C-27; 2-tert-Butyl-4-bromo-5-amino-phenol

To a solution of 2-tert-butyl-4-bromo-5-nitrophenol (C-14-a) (12 g, 43.8 mmol) in MeOH (90 mL) was added Ni (2.4 g). The reaction mixture was stirred under H₂ (1 atm) for 4 h. The mixture was filtered and the filtrate was concentrated. The crude product was recrystallized from ethyl acetate and petroleum ether to give 2-tert-butyl-4-bromo-5-amino-phenol (C-27) (7.2 g, 70%). ¹H NMR (DMSO-d₆) δ 9.15 (s, 1H), 6.91 (s, 1H), 6.24 (s, 1H), 4.90 (br s, 2H), 1.22 (s, 9H); ESI-MS 244.0 m/z (MH⁺).

### Example 13

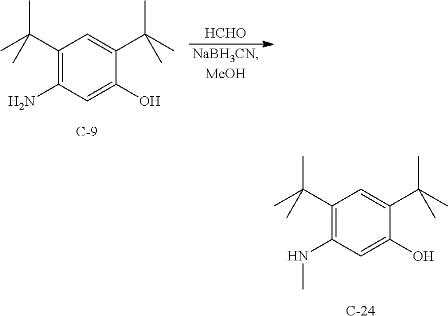

### C-24; 2,4-Di-tert-butyl-6-(N-methylamino)phenol

A mixture of 2,4-di-tert-butyl-6-amino-phenol (C-9) (5.08 g, 23 mmol), NaBH₃CN (4.41 g, 70 mmol) and paraformaldehyde (2.1 g, 70 mmol) in methanol (50 mL) was stirred at reflux for 3 h. After removal of the solvent, the residue was purified by column chromatography (petroleum ether-EtOAc, 30:1) to give 2,4-di-tert-butyl-6-(N-methylamino)phenol (C-24) (800 mg, 15%). ¹H NMR (DMSO-d₆) δ 8.67 (s, 1H), 6.84 (s, 1H), 5.99 (s, 1H), 4.36 (q, J=4.8 Hz, 1H), 2.65 (d, J=4.8 Hz, 3H), 1.23 (s, 18H); ESI-MS 236.2 m/z (MH⁺).

### Example 14

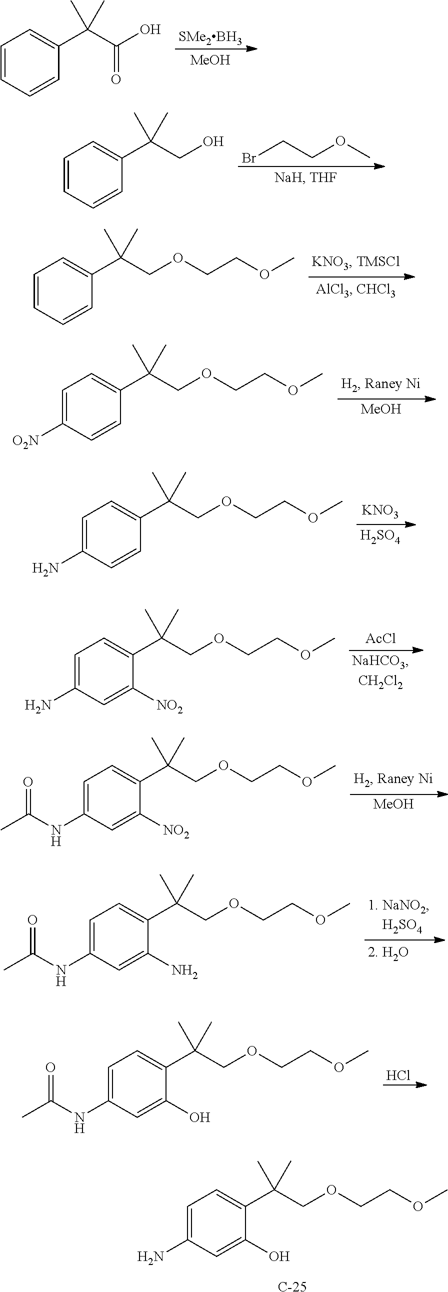

### 2-Methyl-2-phenyl-propan-1-ol

To a solution of 2-methyl-2-phenyl-propionic acid (82 g, 0.5 mol) in THF (200 mL) was added dropwise borane-dimethyl sulfide (2M, 100 mL) at 0-5° C. The mixture was stirred at this temperature for 30 min and then heated at reflux for 1 h. After cooling, methanol (150 mL) and water (50 mL) were added. The mixture was extracted with EtOAc (100 mL×3), and the combined organic layers were washed with water and brine, dried over Na₂SO₄ and concentrated to give 2-methyl-2-phenyl-propan-1-ol as an oil (70 g, 77%).

### 2-(2-Methoxy-ethoxy)-1,1-dimethyl-ethyl]-benzene

To a suspension of NaH (29 g, 0.75 mol) in THF (200 mL) was added dropwise a solution of 2-methyl-2-phenyl-propan-1-ol (75 g, 0.5 mol) in THF (50 mL) at 0° C. The mixture was stirred at 20° C. for 30 min and then a solution of 1-bromo-2-methoxy-ethane (104 g, 0.75 mol) in THF (100 mL) was added dropwise at 0° C. The mixture was stirred at 20° C. overnight, poured into water (200 mL) and extracted with EtOAc (100 mL×3). The combined organic layers were washed with water and brine, dried over Na₂SO₄, and concentrated. The residue was purified by column chromatography (silica gel, petroleum ether) to give 2-(2-Methoxy-ethoxy)-1,1-dimethyl-ethyl]-benzene as an oil (28 g, 27%).

### 1-[2-(2-Methoxy-ethoxy)-1,1-dimethyl-ethyl]-4-nitro-benzene

To a solution of 2-(2-methoxy-ethoxy)-1,1-dimethyl-ethyl]-benzene (52 g, 0.25 mol) in CHCl³ (200 mL) was added KNO₃ (50.5 g, 0.5 mol) and TMSCl (54 g, 0.5 mol). The mixture was stirred at 20° C. for 30 min and then AlCl₃ (95 g, 0.7 mol) was added. The reaction mixture was stirred at 20° C. for 1 h and poured into ice-water. The organic layer was separated and the aqueous layer was extracted with CHCl₃ (50 mL×3). The combined organic layers were washed with water and brine, dried over Na₂SO₄, and concentrated. The residue was purified by column chromatography (silica gel, petroleum ether) to obtain 1-[2-(2-methoxy-ethoxy)-1,1-dimethyl-ethyl]-4-nitro-benzene (6 g, 10%).

### 4-[2-(2-Methoxy-ethoxy)-1,1-dimethyl-ethyl]-phenylamine

A suspension of 1-[2-(2-methoxy-ethoxy)-1,1-dimethyl-ethyl]-4-nitro-benzene (8.1 g, 32 mmol) and Raney Ni (1 g) in MeOH (50 mL) was stirred under H₂ (1 atm) at room temperature for 1 h. The catalyst was filtered off and the filtrate was concentrated to obtain 4-[2-(2-methoxy-ethoxy)-1,1-dimethyl-ethyl]-phenylamine (5.5 g, 77%).

### 4-[2-(2-Methoxy-ethoxy)-1,1-dimethyl-ethyl]-3-nitro-phenylamine

To a solution of 4-[2-(2-methoxy-ethoxy)-1,1-dimethyl-ethyl]-phenylamine (5.8 g, 26 mmol) in H₂SO₄ (20 mL) was added KNO₃ (2.63 g, 26 mmol) at 0° C. After addition was complete, the mixture was stirred at this temperature for 20 min and then poured into ice-water. The mixture was extracted with EtOAc (50 mL×3). The combined organic layers were washed with water and brine, dried over Na₂SO₄, and concentrated. The residue was purified by column chromatography (petroleum ether-EtOAc, 100:1) to give 4-[2-(2-methoxy-ethoxy)-1,1-dimethyl-ethyl]-3-nitro-phenylamine (5 g, 71%).

### N-{4-[2-(2-Methoxy-ethoxy)-1,1-dimethyl-ethyl]-3-nitro-phenyl}-acetamide

To a suspension of NaHCO₃ (10 g, 0.1 mol) in dichloromethane (50 mL) was added 4-[2-(2-methoxy-ethoxy)-1,1-dimethyl-ethyl]-3-nitro-phenylamine (5 g, 30 mmol) and acetyl chloride (3 mL, 20 mmol) at 0-5° C. The mixture was stirred overnight at 15° C. and then poured into water (200 mL). The organic layer was separated and the aqueous layer was extracted with dichloromethane (50 mL×2). The combined organic layers were washed with water and brine, dried over Na₂SO₄, and concentrated to dryness to give N-{4-[2-(2-methoxy-ethoxy)-1,1-dimethyl-ethyl]-3-nitro-phenyl}-acetamide (5.0 g, 87%).

### N-{3-Amino-4-[2-(2-methoxy-ethoxy)-1,1-dimethyl-ethyl]-phenyl}-acetamide

A mixture of N-{4-[2-(2-methoxy-ethoxy)-1,1-dimethyl-ethyl]-3-nitro-phenyl}-acetamide (5 g, 16 mmol) and Raney Ni (1 g) in MeOH (50 mL) was stirred under H₂ (1 atm) at room temperature 1 h. The catalyst was filtered off and the filtrate was concentrated. The residue was purified by column chromatography (petroleum ether-EtOAc, 100:1) to give N-{3-amino-4-[2-(2-methoxy-ethoxy)-1,1-dimethyl-ethyl]-phenyl}-acetamide (1.6 g, 35%).

### N-{3-Hydroxy-4-[2-(2-methoxy-ethoxy)-1,1-dimethyl-ethyl]-phenyl}-acetamide

To a solution of N-{3-amino-4-[2-(2-methoxy-ethoxy)-1,1-dimethyl-ethyl]-phenyl}-acetamide (1.6 g, 5.7 mmol) in H₂SO₄ (15%, 6 mL) was added NaNO₂ at 0-5° C. The mixture was stirred at this temperature for 20 min and then poured into ice water. The mixture was extracted with EtOAc (30 mL×3). The combined organic layers were washed with water and brine, dried over Na₂SO₄ and concentrated. The residue was purified by column chromatography (petroleum ether-EtOAc, 100:1) to give N-{3-hydroxy-4-[2-(2-methoxy-ethoxy)-1,1-dimethyl-ethyl]-phenyl}-acetamide (0.7 g, 38%).

### C-25; 2-(1-(2-Methoxyethoxy)-2-methylpropan-2-yl)-5-aminophenol

A mixture of N-{3-hydroxy-4-[2-(2-methoxy-ethoxy)-1,1-dimethyl-ethyl]-phenyl}-acetamide (1 g, 3.5 mmol) and HCl (5 mL) was heated at reflux for 1 h. The mixture was basified with Na₂CO₃ solution to pH 9 and then extracted with EtOAc (20 mL×3). The combined organic layers were washed with water and brine, dried over Na₂SO₄ and concentrated to dryness. The residue was purified by column chromatography (petroleum ether-EtOAc, 100:1) to obtain 2-(1-(2-methoxyethoxy)-2-methylpropan-2-yl)-5-aminophenol (C-25) (61 mg, 6%). ¹H NMR (CDCl₃) δ 9.11 (br s, 1H), 6.96-6.98 (d, J=8 Hz, 1H), 6.26-6.27 (d, J=4 Hz, 1H), 6.17-6.19 (m, 1H), 3.68-3.69 (m, 2H), 3.56-3.59 (m, 4H), 3.39 (s, 3H), 1.37 (s, 6H); ESI-MS 239.9 m/z (MH⁺).

### Example 15

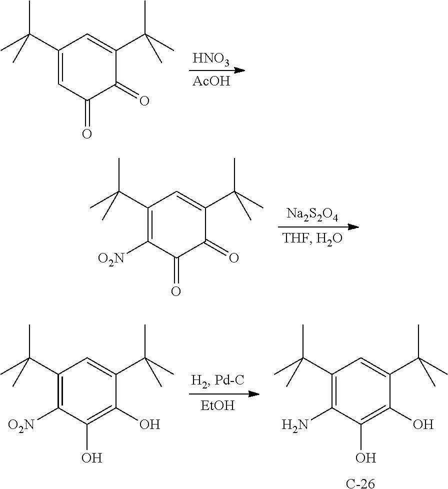

### 4,6-di-tert-Butyl-3-nitrocyclohexa-3,5-diene-1,2-dione

To a solution of 3,5-di-tert-butylcyclohexa-3,5-diene-1,2-dione (4.20 g, 19.1 mmol) in acetic acid (115 mL) was slowly added HNO₃ (15 mL). The mixture was heated at 60° C. for 40 min before it was poured into H₂O (50 mL). The mixture was allowed to stand at room temperature for 2 h, then was placed in an ice bath for 1 h. The solid was collected and washed with water to provide 4,6-di-tert-butyl-3-nitrocyclohexa-3,5-diene-1,2-dione (1.2 g, 24%). ¹H NMR (400 MHz, DMSO-d₆) δ 6.89 (s, 1H), 1.27 (s, 9H), 1.24 (s, 9H).

### 4,6-Di-tert-butyl-3-nitrobenzene-1,2-diol

In a separatory funnel was placed THF/H₂O (1:1, 400 mL), 4,6-di-tert-butyl-3-nitrocyclohexa-3,5-diene-1,2-dione (4.59 g, 17.3 mmol) and Na₂S₂O₄ (3 g, 17.3 mmol). The separatory funnel was stoppered and was shaken for 2 min. The mixture was diluted with EtOAc (20 mL). The layers were separated and the organic layer was washed with brine, dried over MgSO₄ and concentrated to provide 4,6-di-tert-butyl-3-nitrobenzene-1,2-diol (3.4 g, 74%), which was used without further purification. ¹H NMR (400 MHz, DMSO-d₆) δ 9.24 (s, 1H), 8.76 (s, 1H), 6.87 (s, 1H), 1.35 (s, 9H), 1.25 (s, 9H).

### C-26; 4,6-Di-tert-butyl-3-aminobenzene-1,2-diol

To a solution of 4,6-di-tert-butyl-3-nitrobenzene-1,2-diol (1.92 g, 7.2 mmol) in EtOH (70 mL) was added Pd-5% wt. on carbon (200 mg). The mixture was stirred under H₂ (1 atm) for 2 h. The reaction was recharged with Pd-5% wt. on carbon (200 mg) and stirred under H₂ (1 atm) for another 2 h. The mixture was filtered through Celite and the filtrate was concentrated and purified by column chromatography (10-40% ethyl acetate-hexanes) to give 4,6-di-tert-butyl-3-aminobenzene-1,2-diol (C-26) (560 mg, 33%). ¹H NMR (400 MHz, CDCl₃) δ 7.28 (s, 1H), 1.42 (s, 9H), 1.38 (s, 9H).

### Anilines

### Example 1

### General Scheme

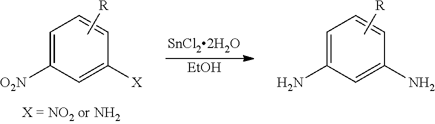

### Specific Example

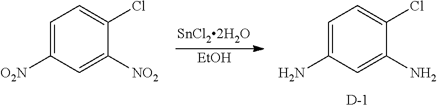

### D-1; 4-Chloro-benzene-1,3-diamine

A mixture of 1-chloro-2,4-dinitro-benzene (100 mg, 0.5 mmol) and SnCl₂.2H₂O (1.12 g, 5 mmol) in ethanol (2.5 mL) was stirred at room temperature overnight. Water was added and then the mixture was basified to pH 7-8 with saturated NaHCO₃ solution. The solution was extracted with ethyl acetate. The combined organic layers were washed with brine, dried over Na₂SO₄, filtered and concentrated to yield 4-chloro-benzene-1,3-diamine (D-1) (79 mg, quant.). HPLC ret. time 0.38 min, 10-99% CH₃CN, 5 min run; ESI-MS 143.1 m/z (MH⁺)

### Other Examples

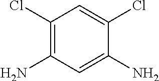

### D-2; 4,6-Dichloro-benzene-1,3-diamine

4,6-Dichloro-benzene-1,3-diamine (D-2) was synthesized following the general scheme above starting from 1,5-dichloro-2,4-dinitro-benzene. Yield (95%). HPLC ret. time 1.88 min, 10-99% CH₃CN, 5 min run; ESI-MS 177.1 m/z (MH⁺).

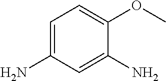

### D-3; 4-Methoxy-benzene-1,3-diamine

4-Methoxy-benzene-1,3-diamine (D-3) was synthesized following the general scheme above starting from 1-methoxy-2,4-dinitro-benzene. Yield (quant.). HPLC ret. time 0.31 min, 10-99% CH₃CN, 5 min run.

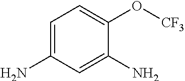 [SMILES: Nc1ccc(OC(F)(F)F)c(N)c1]

### D-4; 4-Trifluoromethoxy-benzene-1,3-diamine

4-Trifluoromethoxy-benzene-1,3-diamine (D-4) was synthesized following the general scheme above starting from 2,4-dinitro-1-trifluoromethoxy-benzene. Yield (89%). HPLC ret. time 0.91 min, 10-99% CH₃CN, 5 min run; ESI-MS 193.3 m/z (MH⁺).

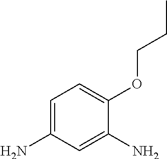

### D-5; 4-Propoxybenzene-1,3-diamine

4-Propoxybenzene-1,3-diamine (D-5) was synthesized following the general scheme above starting from 5-nitro-2-propoxy-phenylamine. Yield (79%). HPLC ret. time 0.54 min, 10-99% CH₃CN, 5 min run; ESI-MS 167.5 m/z (MH⁺).

### Example 2

### General Scheme

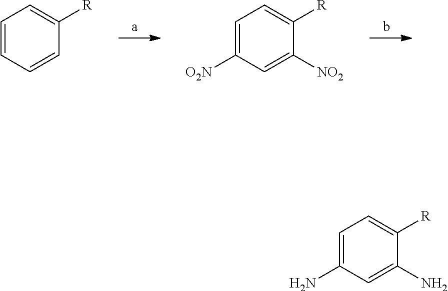

a) HNO₃, H₂SO₄; b) SnCl₂.2H₂O, EtOH or H₂, Pd—C, MeOH

### Specific Example

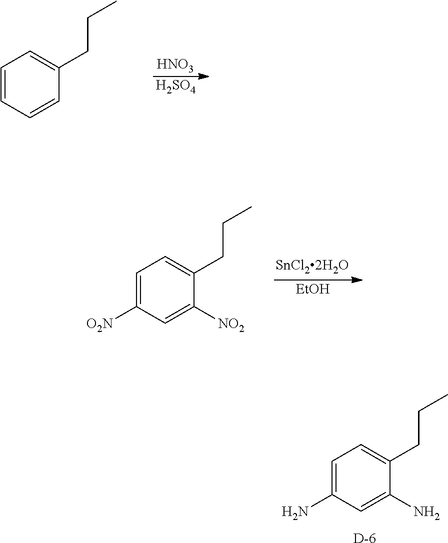

### 2,4-Dinitro-propylbenzene

A solution of propylbenzene (10 g, 83 mmol) in conc. H₂SO₄ (50 mL) was cooled at 0° C. for 30 min, and a solution of conc. H₂SO₄ (50 mL) and fuming HNO₃ (25 mL), previously cooled to 0° C., was added in portions over 15 min. The mixture was stirred at 0° C. for additional 30 min, and then allowed to warm to room temperature. The mixture was poured into ice (200 g)-water (100 mL) and extracted with ether (2×100 mL). The combined extracts were washed with H₂O (100 mL) and brine (100 mL), dried over MgSO₄, filtered and concentrated to afford 2,4-dinitro-propylbenzene (15.6 g, 89%). ¹H NMR (CDCl₃, 300 MHz) δ 8.73 (d, J=2.2 Hz, 1H), 8.38 (dd, J=8.3, J=2.2, 1H), 7.6 (d, J=8.5 Hz, 1H), 2.96 (dd, 2H), 1.73 (m, 2H), 1.06 (t, J=7.4 Hz, 3H).

### D-6; 4-Propyl-benzene-1,3-diamine

To a solution of 2,4-dinitro-propylbenzene (2.02 g, 9.6 mmol) in ethanol (100 mL) was added SnCl₂ (9.9 g, 52 mmol) followed by conc. HCl (10 mL). The mixture was refluxed for 2 h, poured into ice-water (100 mL), and neutralized with solid sodium bicarbonate. The solution was further basified with 10% NaOH solution to pH ˜10 and extracted with ether (2×100 mL). The combined organic layers were washed with brine (100 mL), dried over MgSO₄, filtered, and concentrated to provide 4-propyl-benzene-1,3-diamine (D-6) (1.2 g, 83%). No further purification was necessary for use in the next step; however, the product was not stable for an extended period of time. ¹H NMR (CDCl₃, 300 MHz) δ 6.82 (d, J=7.9 Hz, 1H), 6.11 (dd, J=7.5, J=2.2 Hz, 1H), 6.06 (d, J=2.2 Hz, 1H), 3.49 (br s, 4H, NH₂), 2.38 (t, J=7.4 Hz, 2H), 1.58 (m, 2H), 0.98 (t, J=7.2 Hz, 3H); ESI-MS 151.5 m/z (MH⁺).

### Other Examples

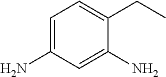

### D-7; 4-Ethylbenzene-1,3-diamine

4-Ethylbenzene-1,3-diamine (D-7) was synthesized following the general scheme above starting from ethylbenezene. Overall yield (76%).

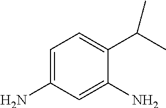

### D-8; 4-Isopropylbenzene-1,3-diamine

4-Isopropylbenzene-1,3-diamine (D-8) was synthesized following the general scheme above starting from isopropylbenezene. Overall yield (78%).

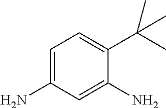

### D-9; 4-tert-Butylbenzene-1,3-diamine

4-tert-Butylbenzene-1,3-diamine (D-9) was synthesized following the general scheme above starting from tert-butylbenzene. Overall yield (48%). ¹H NMR (400 MHz, CDCl₃) δ 7.01 (4, J=8.3 Hz, 1H), 6.10 (dd, J=2.4, 8.3 Hz, 1H), 6.01 (d, J=2.4 Hz, 1H), 3.59 (br, 4H), 1.37 (s, 9H); ¹³C NMR (100 MHz, CDCl₃) δ 145.5, 145.3, 127.6, 124.9, 105.9, 104.5, 33.6, 30.1; ESI-MS 164.9 m/z (MH⁺).

### Example 3

### General Scheme

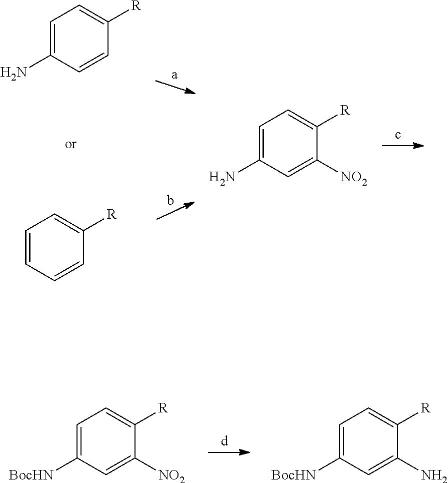

a) KNO₃, H₂SO₄; b) (i) HNO₃, H₂SO₄; (ii) Na₂S, S, H₂O; c) Boc₂O, NaOH, THF; d) H₂, Pd—C, MeOH

### Specific Example

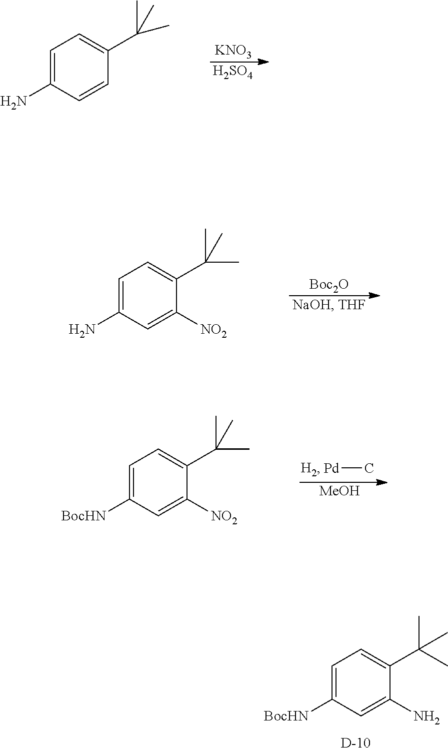

### 4-tert-Butyl-3-nitro-phenylamine

To a mixture of 4-tert-butyl-phenylamine (10.0 g, 67.01 mmol) dissolved in H₂SO₄ (98%, 60 mL) was slowly added KNO₃ (8.1 g, 80.41 mmol) at 0° C. After addition, the reaction was allowed to warm to room temperature and stirred overnight. The mixture was then poured into ice-water and basified with saturated NaHCO₃ solution to pH 8. The mixture was extracted several times with CH₂Cl₂. The combined organic layers were washed with brine, dried over Na₂SO₄ and concentrated. The residue was purified by column chromatography (petroleum ether-EtOAc, 10:1) to give 4-tert-butyl-3-nitro-phenylamine (10 g, 77%).

### (4-tert-Butyl-3-nitro-phenyl)-carbamic acid tert-butyl ester

A mixture of 4-tert-butyl-3-nitro-phenylamine (4.0 g, 20.6 mmol) and Boc₂O (4.72 g, 21.6 mmol) in NaOH (2N, 20 mL) and THF (20 mL) was stirred at room temperature overnight. THF was removed under reduced pressure. The residue was dissolved in water and extracted with CH₂Cl₂. The organic layer was washed with NaHCO₃ and brine, dried over Na₂SO₄ and concentrated to afford (4-tert-butyl-3-nitro-phenyl)-carbamic acid tert-butyl ester (4.5 g, 74%).

### D-10; (3-Amino-4-tert-butyl-phenyl)-carbamic acid tert-butyl ester

A suspension of (4-tert-butyl-3-nitro-phenyl)-carbamic acid tert-butyl ester (3.0 g, 10.19 mol) and 10% Pd—C (1 g) in MeOH (40 mL) was stirred under H₂ (1 atm) at room temperature overnight. After filtration, the filtrate was concentrated and the residue was purified by column chromatograph (petroleum ether-EtOAc, 5:1) to give (3-amino-4-tert-butyl-phenyl)-carbamic acid tert-butyl ester (D-10) as a brown oil (2.5 g, 93%). ¹H NMR (CDCl₃) δ 7.10 (d, J=8.4 Hz, 1H), 6.92 (s, 1H), 6.50-6.53 (m, 1H), 6.36 (s, 1H), 3.62 (br s, 2H), 1.50 (s, 9H), 1.38 (s, 9H); ESI-MS 528.9 m/z (2M+H⁺).

### Other Examples

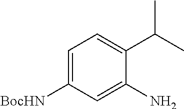 [SMILES: CC(C)c1ccc(NC(=O)OC(C)(C)C)cc1N]

### D-11; (3-Amino-4-isopropyl-phenyl)-carbamic acid tert-butyl ester

(3-Amino-4-isopropyl-phenyl)-carbamic acid tert-butyl ester (D-11) was synthesized following the general scheme above starting from isopropylbenezene. Overall yield (56%).

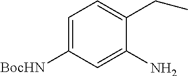 [SMILES: CCc1ccc(NC(=O)OC(C)(C)C)cc1N]

### D-12; (3-Amino-4-ethyl-phenyl)-carbamic acid tert-butyl ester

(3-Amino-4-ethyl-phenyl)-carbamic acid tert-butyl ester (D-12) was synthesized following the general scheme above starting from ethylbenezene. Overall yield (64%). ¹H NMR (CD₃OD, 300 MHz) δ 6.87 (d, J=8.0 Hz, 1H), 6.81 (d, J=2.2 Hz, 1H), 6.63 (dd, J=8.1, J=2.2, 1H), 2.47 (q, J=7.4 Hz, 2H), 1.50 (s, 9H), 1.19 (t, J=7.4 Hz, 3H); ESI-MS 237.1 m/z (MH⁺).

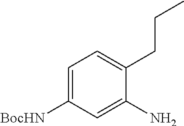 [SMILES: CCCc1ccc(NC(=O)OC(C)(C)C)cc1N]

### D-13; (3-Amino-4-propyl-phenyl)-carbamic acid tert-butyl ester

(3-Amino-4-propyl-phenyl)-carbamic acid tert-butyl ester (D-13) was synthesized following the general scheme above starting from propylbenezene. Overall yield (48%).

### Example 4

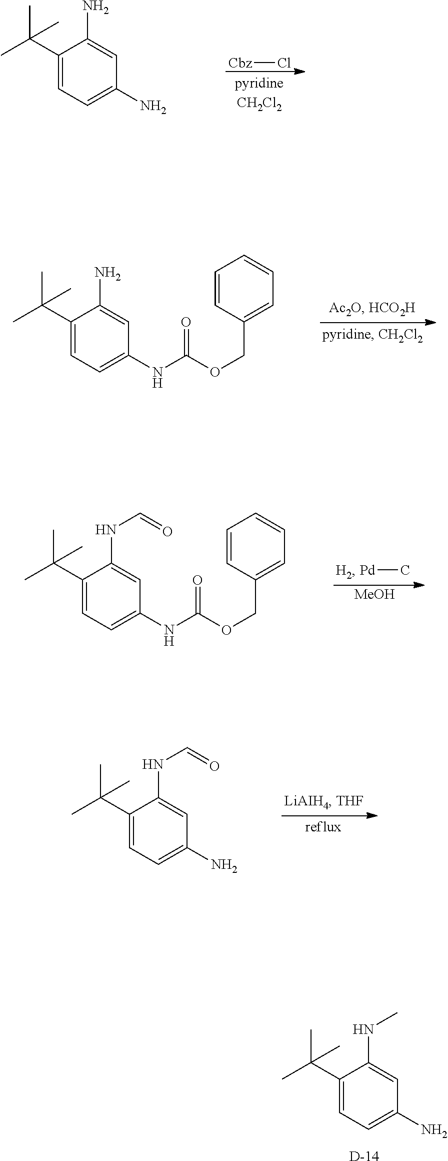

### (3-Amino-4-tert-butyl-phenyl)-carbamic acid benzyl ester

A solution of 4-tert-butylbenzene-1,3-diamine (D-9) (657 mg, 4 mmol) and pyridine (0.39 mL, 4.8 mmol) in CH₂Cl₂/MeOH (12/1, 8 mL) was cooled to 0° C., and a solution of benzyl chloroformate (0.51 mL, 3.6 mmol) in CH₂Cl₂ (8 mL) was added dropwise over 10 min. The mixture was stirred at 0° C. for 15 min, then warmed to room temperature. After 1 h, the mixture was washed with 1M citric acid (2×20 mL), saturated aqueous sodium bicarbonate (20 mL), dried (Na₂SO₄), filtered and concentrated in vacuo to afford the crude (3-amino-4-tert-butyl-phenyl)-carbamic acid benzyl ester as a brown viscous gum (0.97 g), which was used without further purification. ¹H NMR (400 MHz, CDCl₃) δ 7.41-7.32 (m, 6H,), 7.12 (d, J=8.5 Hz, 1H), 6.89 (br s, 1H), 6.57 (dd, J=2.3, 8.5 Hz, 1H), 5.17 (s, 2H), 3.85 (br s, 2H), 1.38 (s, 9H); ¹³C NMR (100 MHz, CDCl₃, rotameric) δ 153.3 (br), 145.3, 136.56, 136.18, 129.2, 128.73, 128.59, 128.29, 128.25, 127.14, 108.63 (br), 107.61 (br), 66.86, 33.9, 29.7; ESI-MS 299.1 m/z (MH⁺).

### (4-tert-Butyl-3-formylamino-phenyl)-carbamic acid benzyl ester

A solution of (3-amino-4-tert-butyl-phenyl)-carbamic acid benzyl ester (0.97 g, 3.25 mmol) and pyridine (0.43 mL, 5.25 mmol) in CH₂Cl₂ (7.5 mL) was cooled to 0° C., and a solution of formic-acetic anhydride (3.5 mmol, prepared by mixing formic acid (158 μL, 4.2 mmol, 1.3 equiv) and acetic anhydride (0.32 mL, 3.5 mmol, 1.1 eq.) neat and ageing for 1 hour) in CH₂Cl₂ (2.5 mL) was added dropwise over 2 min. After the addition was complete, the mixture was allowed to warm to room temperature, whereupon it deposited a precipitate, and the resulting slurry was stirred overnight. The mixture was washed with 1 M citric acid (2×20 mL), saturated aqueous sodium bicarbonate (20 mL), dried (Na₂SO₄), and filtered. The cloudy mixture deposited a thin bed of solid above the drying agent, HPLC analysis showed this to be the desired formamide. The filtrate was concentrated to approximately 5 mL, and diluted with hexane (15 mL) to precipitate further formamide. The drying agent (Na₂SO₄) was slurried with methanol (50 mL), filtered, and the filtrate combined with material from the CH₂Cl₂/hexane recrystallisation. The resultant mixture was concentrated to afford (4-tert-butyl-3-formylamino-phenyl)-carbamic acid benzyl ester as an off-white solid (650 mg, 50% over 2 steps). ¹H and ¹³C NMR (CD₃OD) show the product as a rotameric mixture. ¹H NMR (400 MHz, CD₃OD, rotameric) δ 8.27 (s, 1H-a), 8.17 (s, 1H-b), 7.42-7.26 (m, 8H), 5.17 (s, 1H-a), 5.15 (s, 1H-b), 4.86 (s, 2H), 1.37 (s, 9H-a), 1.36 (s, 9H-b) ¹³C NMR (100 MHz, CD₃OD, rotameric) δ 1636.9, 163.5, 155.8, 141.40, 141.32, 139.37, 138.88, 138.22, 138.14, 136.4, 135.3, 129.68, 129.65, 129.31, 129.24, 129.19, 129.13, 128.94, 128.50, 121.4 (br), 118.7 (br), 67.80, 67.67, 35.78, 35.52, 31.65, 31.34; ESI-MS 327.5 m/z (MH⁺).

### N-(5-Amino-2-tert-butyl-phenyl)-formamide

A 100 mL flask was charged with (4-tert-butyl-3-formylamino-phenyl)-carbamic acid benzyl ester (650 mg, 1.99 mmol), methanol (30 mL) and 10% Pd—C (50 mg), and stirred under H₂ (1 atm) for 20 h. CH₂Cl₂ (5 mL) was added to quench the catalyst, and the mixture then filtered through Celite, and concentrated to afford N-(5-amino-2-tert-butyl-phenyl)-formamide as an off-white solid (366 mg, 96%). Rotameric by ¹H and ¹³C NMR (DMSO-d₆). ¹H NMR (400 MHz, DMSO-d₆, rotameric) δ (d, 9.24 J=10.4 Hz, 1H), 9.15 (s, 1H), 8.23 (d, J=1.5 Hz, 1H), 8.06 (d, J=10.4 Hz, 1H), 7.06 (d, J=8.5 Hz, 1H), 7.02 (d, J=8.5 Hz, 1H), 6.51 (d, J=2.5 Hz, 1H), 6.46 (dd, J=2.5, 8.5 Hz, 1H), 6.39 (dd, J=2.5, 8.5 Hz, 1H), 6.29 (d, J=2.5 Hz, 1H), 5.05 (s, 2H), 4.93 (s, 2H), 1.27 (s, 9H); ¹³C NMR (100 MHz, DMSO-d₆, rotameric) δ 164.0, 160.4, 147.37, 146.74, 135.38, 135.72, 132.48, 131.59, 127.31, 126.69, 115.15, 115.01, 112.43, 112.00, 33.92, 33.57, 31.33, 30.92; ESI-MS 193.1 m/z (MH⁺).

### D-14; 4-tert-butyl-N³-methyl-benzene-1,3-diamine

A 100 mL flask was charged with N-(5-amino-2-tert-butyl-phenyl)-formamide (340 mg, 1.77 mmol) and purged with nitrogen. THF (10 mL) was added, and the solution was cooled to 0° C. A solution of lithium aluminum hydride in THF (4.4 mL, 1M solution) was added over 2 min. The mixture was then allowed to warm to room temperature. After refluxing for 15 h, the yellow suspension was cooled to 0° C., quenched with water (170 μL), 15% aqueous NaOH (170 μL), and water (510 μL) which were added sequentially and stirred at room temperature for 30 min. The mixture was filtered through Celite, and the filter cake washed with methanol (50 mL). The combined filtrates were concentrated in vacuo to give a gray-brown solid, which was partitioned between chloroform (75 mL) and water (50 mL). The organic layer was separated, washed with water (50 mL), dried (Na₂SO₄), filtered, and concentrated to afford 4-tert-butyl-N³-methyl-benzene-1,3-diamine (D-14) as a brown oil which solidified on standing (313 mg, 98%). ¹H NMR (400 MHz, CDCl₃) δ 7.01 (4, J=8.1 Hz, 1H), 6.05 (dd, J=2.4, 8.1 Hz, 1H), 6.03 (d, J=2.4 Hz, 1H), 3.91 (br s, 1H), 3.52 (br s, 2H), 2.86 (s, 3H), 1.36 (s, 9H); ¹³C NMR (100 MHz, CDCl₃) δ 148.4, 145.7, 127.0, 124.3, 103.6, 98.9, 33.5, 31.15, 30.31; ESI-MS 179.1 m/z (MH⁺).

### Example 5

### General Scheme

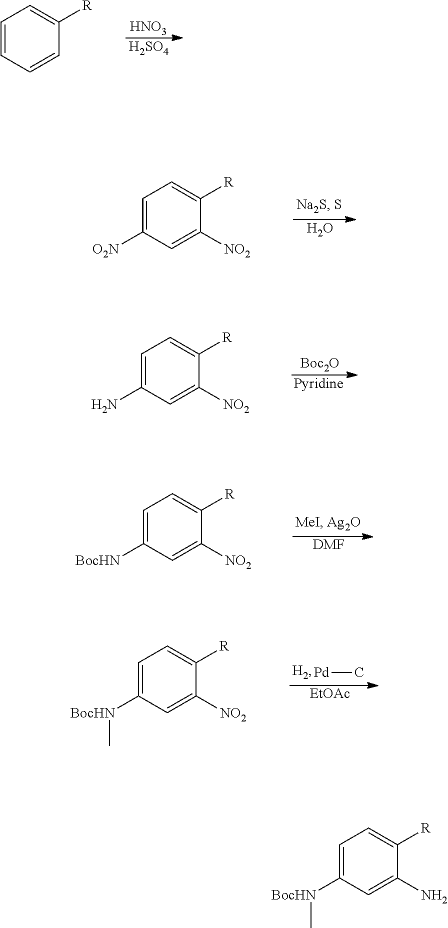

### Specific Example

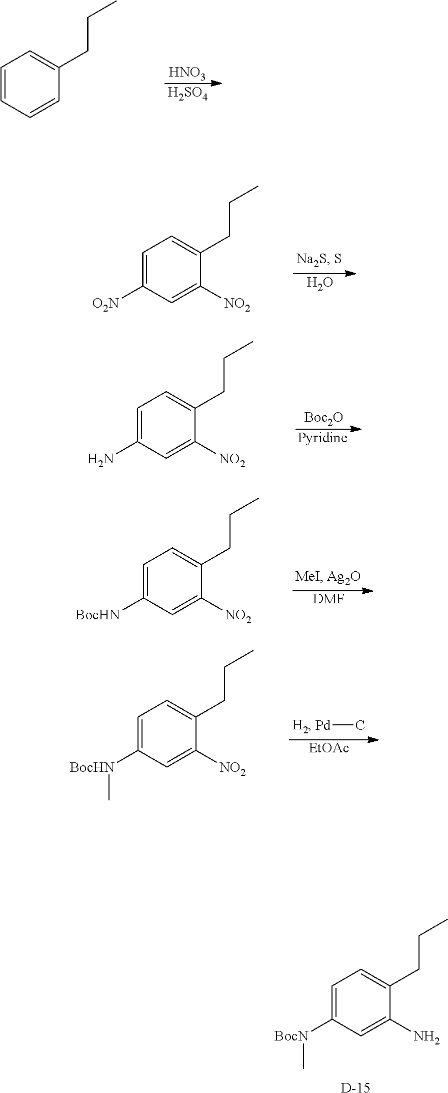

### 2,4-Dinitro-propylbenzene

A solution of propylbenzene (10 g, 83 mmol) in conc. H₂SO₄ (50 mL) was cooled at 0° C. for 30 mins, and a solution of conc. H₂SO₄ (50 mL) and fuming HNO₃ (25 mL), previously cooled to 0° C., was added in portions over 15 min. The mixture was stirred at 0° C. for additional 30 min. and then allowed to warm to room temperature. The mixture was poured into ice (200 g)-water (100 mL) and extracted with ether (2×100 mL). The combined extracts were washed with H₂O (100 mL) and brine (100 mL), dried over MgSO₄, filtered and concentrated to afford 2,4-dinitro-propylbenzene (15.6 g, 89%). ¹H NMR (CDCl₃, 300 MHz) δ 8.73 (d, J=2.2 Hz, 1H), 8.38 (dd, J=8.3, 2.2 Hz, 1H), 7.6 (d, J=8.5 Hz, 1H), 2.96 (m, 2H), 1.73 (m, 2H), 1.06 (t, J=7.4 Hz, 3H).

### 4-Propyl-3-nitroaniline

A suspension of 2,4-dinitro-propylbenzene (2 g, 9.5 mmol) in H₂O (100 mL) was heated near reflux and stirred vigorously. A clear orange-red solution of polysulfide (300 mL (10 eq.), previously prepared by heating sodium sulfide nanohydrate (10.0 g), sulfur powder (2.60 g) and H₂O (400 mL), was added dropwise over 45 mins. The red-brown solution was heated at reflux for 1.5 h. The mixture was cooled to 0° C. and then extracted with ether (2×200 mL). The combined organic extracts were dried over MgSO₄, filtered, and concentrated under reduced pressure to afford 4-propyl-3-nitroaniline (1.6 g, 93%), which was used without further purification.

### (3-Nitro-4-propyl-phenyl)-carbamic acid tert-butyl ester

4-Propyl-3-nitroaniline (1.69 g, 9.4 mmol) was dissolved in pyridine (30 mL) with stirring. Boc anhydride (2.05 g, 9.4 mmol) was added. The mixture was stirred and heated at reflux for 1 h before the solvent was removed in vacuo. The oil obtained was re-dissolved in CH₂Cl₂ (300 mL) and washed with water (300 mL) and brine (300 mL), dried over Na₂SO₄, filtered, and concentrated. The crude oil that contained both mono- and bis-acylated nitro products was purified by column chromatography (0-10% CH₂Cl₂-MeOH) to afford (3-nitro-4-propyl-phenyl)-carbamic acid tert-butyl ester (2.3 g, 87%).

### Methyl-(3-nitro-4-propyl-phenyl)-carbamic acid tert-butyl ester

To a solution of (3-nitro-4-propyl-phenyl)-carbamic acid tert-butyl ester (200 mg, 0.71 mmol) in DMF (5 mL) was added Ag₂O (1.0 g, 6.0 mmol) followed by methyl iodide (0.20 mL, 3.2 mmol). The resulting suspension was stirred at room temperature for 18 h and filtered through a pad of Celite. The filter cake was washed with CH₂Cl₂ (10 mL). The filtrate was concentrated in vacuo. The crude oil was purified by column chromatography (0-10% CH₂Cl₂-MeOH) to afford methyl-(3-nitro-4-propyl-phenyl)-carbamic acid tert-butyl ester as a yellow oil (110 mg, 52%). ¹H NMR (CDCl₃, 300 MHz) δ 7.78 (d, J=2.2 Hz, 1H), 7.42 (dd, J=8.2, 2.2 Hz, 1H), 7.26 (d, J=8.2 Hz, 1H), 3.27 (s, 3H), 2.81 (t, J=7.7 Hz, 2H), 1.66 (m, 2H), 1.61 (s, 9H), 0.97 (t, J=7.4 Hz, 3H).

### D-15; (3-Amino-4-propyl-phenyl)-methyl-carbamic acid tert-butyl ester

To a solution of methyl-(3-nitro-4-propyl-phenyl)-carbamic acid tert-butyl ester (110 mg, 0.37 mmol) in EtOAc (10 ml) was added 10% Pd—C (100 mg). The resulting suspension was stirred at room temperature under H₂ (1 atm) for 2 days. The progress of the reaction was monitored by TLC. Upon completion, the reaction mixture was filtered through a pad of Celite. The filtrate was concentrated in vacuo to afford (3-Amino-4-propyl-phenyl)-methyl-carbamic acid tert-butyl ester (D-15) as a colorless crystalline compound (80 mg, 81%). ESI-MS 265.3 m/z (MH⁺).

### Other Examples

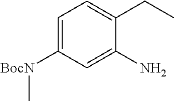 [SMILES: CCc1ccc(N(C)C(=O)OC(C)(C)C)cc1N]

### D-16; (3-Amino-4-ethyl-phenyl)-methyl-carbamic acid tert-butyl ester

(3-Amino-4-ethyl-phenyl)-methyl-carbamic acid tert-butyl ester (D-16) was synthesized following the general scheme above starting from ethylbenezene. Overall yield (57%).

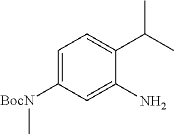 [SMILES: CC(C)c1ccc(N(C)C(=O)OC(C)(C)C)cc1N]

### D-17; (3-Amino-4-isopropyl-phenyl)-methyl-carbamic acid tert-butyl ester

(3-Amino-4-isopropyl-phenyl)-methyl-carbamic acid tert-butyl ester (D-17) was synthesized following the general scheme above starting from isopropylbenezene. Overall yield (38%).

### Example 6

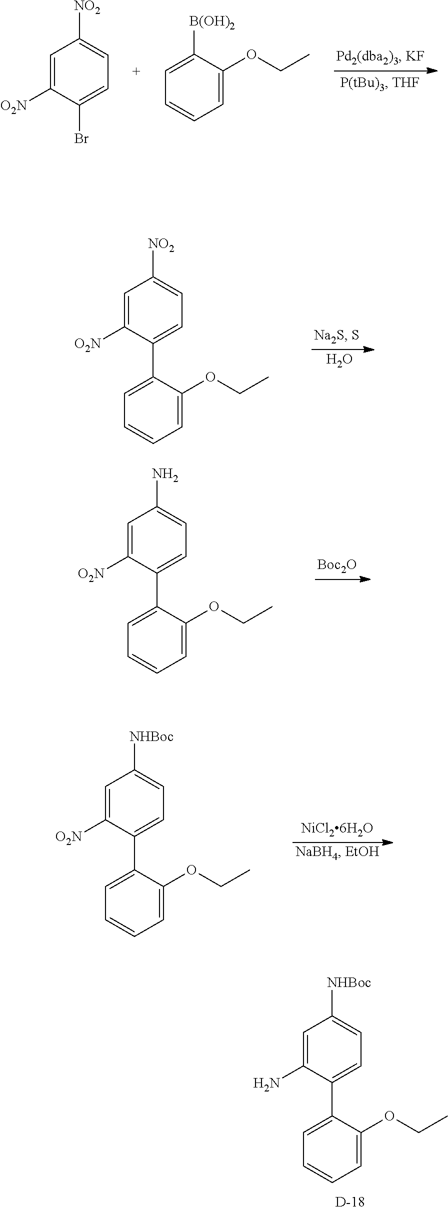

### 2′-Ethoxy-2,4-dinitro-biphenyl

A pressure flask was charged with 2-ethoxyphenylboronic acid (0.66 g, 4.0 mmol), KF (0.77 g, 13 mmol), Pd₂(dba)₃ (16 mg, 0.02 mmol), and 2,4-dinitro-bromobenzene (0.99 g, 4.0 mmol) in THF (5 mL). The vessel was purged with argon for 1 min followed by the addition of tri-tert-butylphosphine (0.15 ml, 0.48 mmol, 10% solution in hexanes). The reaction vessel was purged with argon for additional 1 min., sealed and heated at 80° C. overnight. After cooling to room temperature, the solution was filtered through a plug of Celite. The filter cake was rinsed with CH₂Cl₂ (10 mL), and the combined organic extracts were concentrated under reduced pressure to provide the crude product 2′-ethoxy-2,4-dinitro-biphenyl (0.95 g, 82%). No further purification was performed. ¹H NMR (300 MHz, CDCl₃) δ 8.75 (s, 1H), 8.43 (d, J=8.7 Hz, 1H), 7.60 (d, J=8.4 Hz, 1H), 7.40 (t, J=7.8 Hz, 1H), 7.31 (d, J=7.5 Hz, 1H), 7.08 (t, J=7.5 Hz, 1H), 6.88 (d, J=8.4 Hz, 1H), 3.44 (q, J=6.6 Hz, 2H), 1.24 (t, J=6.6 Hz, 3H); HPLC ret. time 3.14 min, 10-100% CH₃CN, 5 min gradient.

### 2′-Ethoxy-2-nitrobiphenyl-4-yl amine

A clear orange-red solution of polysulfide (120 ml, 7.5 eq.), previously prepared by heating sodium sulfide monohydrate (10 g), sulfur (1.04 g) and water (160 ml), was added dropwise at 90° C. over 45 minutes to a suspension of 2′-ethoxy-2,4-dinitro-biphenyl (1.2 g, 4.0 mmol) in water (40 ml). The red-brown solution was heated at reflux for 1.5 h. The mixture was cooled to room temperature, and solid NaCl (5 g) was added. The solution was extracted with CH₂Cl₂ (3×50 mL), and the combined organic extracts was concentrated to provide 2′-ethoxy-2-nitrobiphenyl-4-yl amine (0.98 g, 95%) that was used in the next step without further purification. ¹H NMR (300 MHz, CDCl₃) δ 7.26 (m, 2H), 7.17 (d, J=2.7 Hz, 1H), 7.11 (d, J=7.8 Hz, 1H), 7.00 (t, J=6.9 Hz, 1H), 6.83 (m, 2H), 3.91 (q, J=6.9 Hz, 2H), 1.23 (t, J=7.2 Hz, 3H); HPLC ret. time 2.81 min, 10-100% CH₃CN, 5 min gradient; ESI-MS 259.1 m/z (MH⁺).

### (2′-Ethoxy-2-nitrobiphenyl-4-yl)-carbamic acid tert-butyl ester

A mixture of 2′-ethoxy-2-nitrobipenyl-4-yl amine (0.98 g, 4.0 mmol) and Boc₂O (2.6 g, 12 mmol) was heated with a heat gun. Upon the consumption of the starting material as indicated by TLC, the crude mixture was purified by flash chromatography (silica gel, CH₂Cl₂) to provide (2′-ethoxy-2-nitrobiphenyl-4-yl)-carbamic acid tert-butyl ester (1.5 g, 83%). ¹H NMR (300 MHz, CDCl₃) δ 7.99 (s, 1H), 7.55 (d, J=8.4 Hz, 1H), 7.25 (m, 3H), 6.99 (t, J=7.5 Hz, 1H), 6.82 (m, 2H), 3.88 (q, J=6.9 Hz, 2H), 1.50 (s, 9H), 1.18 (t, J=6.9 Hz, 3H); HPLC ret. time 3.30 min, 10-100% CH₃CN, 5 min gradient.

### D-18; (2′-ethoxy-2-aminobiphenyl-4-yl)-carbamic acid tert-butyl ester

To a solution of NiCl₂.6H₂O (0.26 g, 1.1 mmol) in EtOH (5 mL) was added NaBH₄ (40 mg, 1.1 mmol) at −10° C. Gas evolution was observed and a black precipitate was formed. After stirring for 5 min, a solution of 2′-ethoxy-2-nitrobiphenyl-4-yl)carbamic acid tert-butyl ester (0.50 g, 1.1 mmol) in EtOH (2 mL) was added. Additional NaBH₄ (80 mg, 60 mmol) was added in 3 portions over 20 min. The reaction was stirred at 0° C. for 20 min followed by the addition of NH₄OH (4 mL, 25% aq. solution). The resulting solution was stirred for 20 min. The crude mixture was filtered through a short plug of silica. The silica cake was flushed with 5% MeOH in CH₂Cl₂ (10 mL), and the combined organic extracts was concentrated under reduced pressure to provide (2′-ethoxy-2-aminobiphenyl-4-yl)-carbamic acid tert-butyl ester (D-18) (0.36 g, quant.), which was used without further purification. HPLC retention time 2.41 min, 10-100% CH₃CN, 5 min gradient; ESI-MS 329.3 m/z (MH⁺).

### Example 7

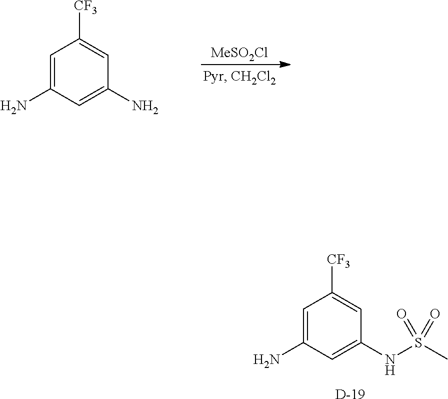

### D-19; N-(3-Amino-5-trifluoromethyl-phenyl)methanesulfonamide

A solution of 5-trifluoromethyl-benzene-1,3-diamine (250 mg, 1.42 mmol) in pyridine (0.52 mL) and CH₂Cl₂ (6.5 mL) was cooled to 0° C. Methanesulfonyl chloride (171 mg, 1.49 mmol) was slowly added at such a rate that the temperature of the solution remained below 10° C. The mixture was stirred at ˜8° C. and then allowed to warm to room temperature after 30 min. After stirring at room temperature for 4 h, reaction was almost complete as indicated by LCMS analysis. The reaction mixture was quenched with sat. aq. NH₄Cl (10 mL) solution, extracted with CH₂Cl₂ (4×10 mL), dried over Na₂SO₄, filtered, and concentrated to yield N-(3-amino-5-trifluoromethyl-phenyl)-methanesulfonamide (D-19) as a reddish semisolid (0.35 g, 97%), which was used without further purification. ¹H-NMR (CDCl₃, 300 MHz) δ 6.76 (m, 1H), 6.70 (m, 1H), 6.66 (s, 1H), 3.02 (s, 3H); ESI-MS 255.3 m/z (MH⁺).

### Cyclic Amines

### Example 1

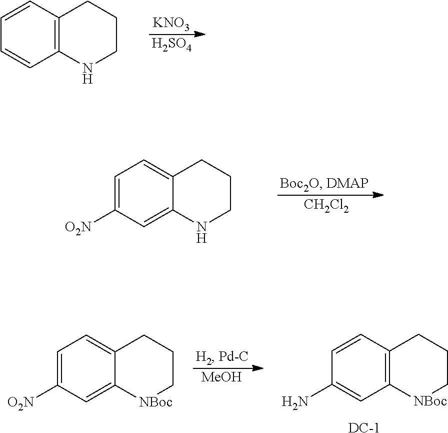

### 7-Nitro-1,2,3,4-tetrahydro-quinoline

To a mixture of 1,2,3,4-tetrahydro-quinoline (20.0 g, 0.15 mol) dissolved in H₂SO₄ (98%, 150 mL), KNO₃ (18.2 g, 0.18 mol) was slowly added at 0° C. The reaction was allowed to warm to room temperature and stirred over night. The mixture was then poured into ice-water and basified with sat. NaHCO₃ solution to pH 8. After extraction with CH₂Cl₂, the combined organic layers were washed with brine, dried over Na₂SO₄ and concentrated. The residue was purified by column chromatography (petroleum ether-EtOAc, 10:1) to give 7-nitro-1,2,3,4-tetrahydro-quinoline (6.6 g, 25%).

### 7-Nitro-3,4-dihydro-2H-quinoline-1-carboxylic acid tert-butyl ester

A mixture of 7-nitro-1,2,3,4-tetrahydro-quinoline (4.0 g, 5.61 mmol), Boc₂O (1.29 g, 5.89 mmol) and DMAP (0.4 g) in CH₂Cl₂ was stirred at room temperature overnight. After diluted with water, the mixture was extracted with CH₂Cl₂. The combined organic layers were washed with NaHCO₃ and brine, dried over Na₂SO₄ and concentrated to provide crude 7-nitro-3,4-dihydro-2H-quinoline-1-carboxylic acid tert-butyl ester that was used in the next step without further purification.

### DC-1; tert-Butyl 7-amino-3,4-dihydroquinoline-1(2H)-carboxylate

A suspension of the crude 7-nitro-3,4-dihydro-2H-quinoline-1-carboxylic acid tert-butyl ester (4.5 g, 16.2 mol) and 10% Pd—C (0.45 g) in MeOH (40 mL) was stirred under H₂ (1 atm) at room temperature overnight. After filtration, the filtrate was concentrated and the residue was purified by column chromatography (petroleum ether-EtOAc, 5:1) to give tert-butyl 7-amino-3,4-dihydroquinoline-1(2H)-carboxylate (DC-1) as a brown solid (1.2 g, 22% over 2 steps). ¹H NMR (CDCl₃) δ 7.15 (d, J=2Hz, 1H), 6.84 (d, J=8 Hz, 1H), 6.36-6.38 (m, 1H), 3.65-3.68 (m, 2H), 3.10 (br s, 2H), 2.66 (t, J=6.4 Hz, 2H), 1.84-1.90 (m, 2H), 1.52 (s, 9H); ESI-MS 496.8 m/z (2M+H⁺).

### Example 2

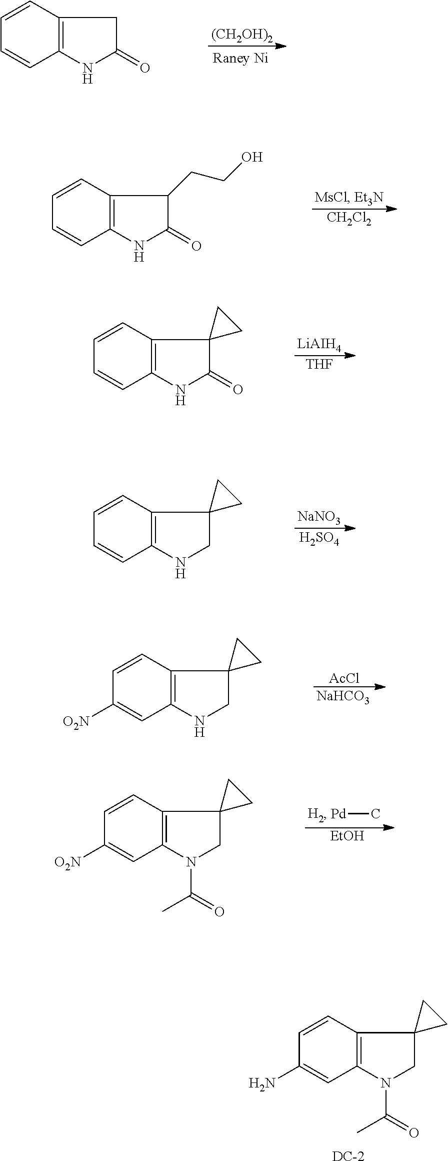

### 3-(2-Hydroxy-ethyl)-1,3-dihydro-indol-2-one

A stirring mixture of oxindole (5.7 g, 43 mmol) and Raney nickel (10 g) in ethane-1,2-diol (100 mL) was heated in an autoclave. After the reaction was complete, the mixture was filtered and the excess of diol was removed under vacuum. The residual oil was triturated with hexane to give 3-(2-hydroxy-ethyl)-1,3-dihydro-indol-2-one as a colorless crystalline solid (4.6 g, 70%).

### 1,2-Dihydro-3-spiro-1′-cyclopropyl-1H-indole-2-one

To a solution of 3-(2-hydroxy-ethyl)-1,3-dihydro-indol-2-one (4.6 g, 26 mmol) and triethylamine (10 mL) in CH₂Cl₂ (100 mL) was added MsCl (3.4 g, 30 mmol) dropwise at −20° C. The mixture was then allowed to warm up to room temperature and stirred overnight. The mixture was filtered and the filtrate was concentrated under vacuum. The residue was purified by column chromatography to give crude 1,2-dihydro-3-spiro-1′-cyclopropyl-1H-indole-2-one as a yellow solid (2.5 g), which was used directly in the next step.

### 1,2-Dihydro-3-spiro-1′-cyclopropyl-1H-indole

To a solution of 1,2-dihydro-3-spiro-1′-cyclopropyl-1H-indole-2-one (2.5 g crude) in THF (50 mL) was added LiAlH₄ (2 g, 52 mmol) portionwise. After heating the mixture to reflux, it was poured into crushed ice, basified with aqueous ammonia to pH 8 and extracted with EtOAc. The combined organic layers were washed with brine, dried over Na₂SO₄ and concentrated to give the crude 1,2-dihydro-3-spiro-1′-cyclopropyl-1H-indole as a yellow solid (about 2 g), which was used directly in the next step.

### 6-Nitro-1,2-dihydro-3-spiro-1′-cyclopropyl-1H-indole

To a cooled solution (−5° C. to −10° C.) of NaNO₃ (1.3 g, 15.3 mmol) in H₂SO₄ (98%, 30 mL) was added 1,2-dihydro-3-spiro-1′-cyclopropyl-1H-indole (2 g, crude) dropwise over a period of 20 min. After addition, the reaction mixture was stirred for another 40 min and poured over crushed ice (20 g). The cooled mixture was then basified with NH₄OH and extracted with EtOAc. The organic layer was washed with brine, dried over Na₂SO₄, and concentrated under reduced pressure to yield 6-nitro-1,2-dihydro-3-spiro-1′-cyclopropyl-1H-indole as a dark gray solid (1.3 g)

### 1-Acetyl-6-nitro-1,2-dihydro-3-spiro-1′-cyclopropyl-1H-indole

NaHCO₃ (5 g) was suspended in a solution of 6-nitro-1,2-dihydro-3-spiro-1′-cyclopropyl-1H-indole (1.3 g, crude) in CH₂Cl₂ (50 mL). While stirring vigorously, acetyl chloride (720 mg) was added dropwise. The mixture was stirred for 1 h and filtered. The filtrate was concentrated under vacuum. The residue was purified by flash column chromatography on silica gel to give 1-acetyl-6-nitro-1,2-dihydro-3-spiro-P-cyclopropyl-1H-indole (0.9 g, 15% over 4 steps).

### DC-2; 1-Acetyl-6-amino-1,2-dihydro-3-spiro-1′-cyclopropyl-1H-indole

A mixture of 1-acetyl-6-nitro-1,2-dihydro-3-spiro-1′-cyclopropyl-1H-indole (383 mg, 2 mmol) and Pd—C (10%, 100 mg) in EtOH (50 mL) was stirred at room temperature under H₂ (1 atm) for 1.5 h. The catalyst was filtered off and the filtrate was concentrated under reduced pressure. The residue was treated with HCl/MeOH to give 1-acetyl-6-amino-1,2-dihydro-3-spiro-1′-cyclopropyl-1H-indole (DC-2) (300 mg, 90%) as a hydrochloride salt.

### Example 3

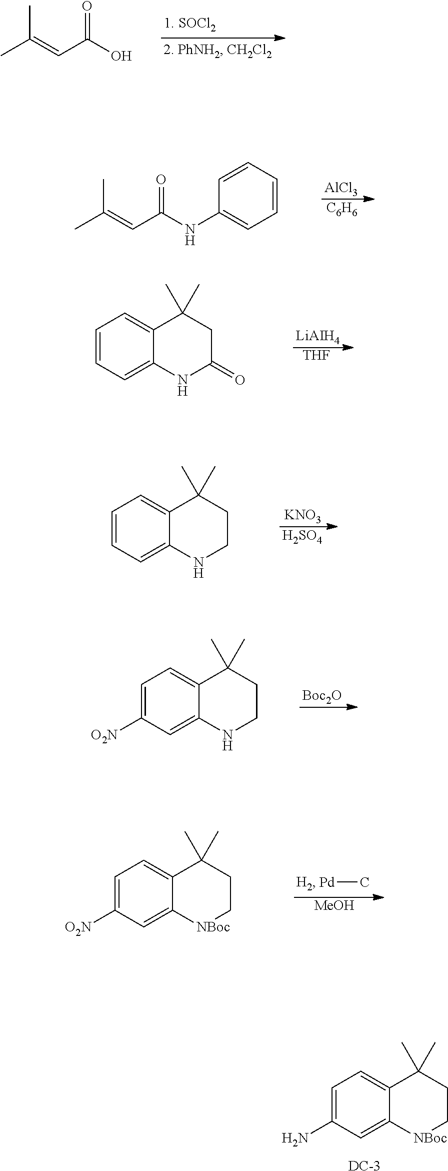

### 3-Methyl-but-2-enoic acid phenylamide

A mixture of 3-methyl-but-2-enoic acid (100 g, 1 mol) and SOCl₂ (119 g, 1 mol) was heated at reflux for 3 h. The excess SOCl₂ was removed under reduced pressure. CH₂Cl₂ (200 mL) was added followed by the addition of aniline (93 g, 1.0 mol) in Et₃N (101 g, 1 mol) at 0° C. The mixture was stirred at room temperature for 1 h and quenched with HCl (5%, 150 mL). The aqueous layer was separated and extracted with CH₂Cl₂. The combined organic layers were washed with water (2×100 mL) and brine (100 mL), dried over Na₂SO₄ and concentrated to give 3-methyl-but-2-enoic acid phenylamide (120 g, 80%).

### 4,4-Dimethyl-3,4-dihydro-1H-quinolin-2-one

AlCl₃ (500 g, 3.8 mol) was carefully added to a suspension of 3-methyl-but-2-enoic acid phenylamide (105 g, 0.6 mol) in benzene (1000 mL). The reaction mixture was stirred at 80° C. overnight and poured into ice-water. The organic layer was separated and the aqueous layer was extracted with ethyl acetate (250 mL×3). The combined organic layers were washed with water (200 mL×2) and brine (200 mL), dried over Na₂SO₄ and concentrated to give 4,4-dimethyl-3,4-dihydro-1H-quinolin-2-one (90 g, 86%).

### 4,4-Dimethyl-1,2,3,4-tetrahydro-quinoline

A solution of 4,4-dimethyl-3,4-dihydro-1H-quinolin-2-one (35 g, 0.2 mol) in THF (100 mL) was added dropwise to a suspension of LiAlH₄ (18 g, 0.47 mol) in THF (200 mL) at 0° C. After addition, the mixture was stirred at room temperature for 30 min and then slowly heated to reflux for 1 h. The mixture was then cooled to 0° C. Water (18 mL) and NaOH solution (10%, 100 mL) were carefully added to quench the reaction. The solid was filtered off and the filtrate was concentrated to give 4,4-dimethyl-1,2,3,4-tetrahydro-quinoline.

### 4,4-Dimethyl-7-nitro-1,2,3,4-tetrahydro-quinoline

To a mixture of 4,4-dimethyl-1,2,3,4-tetrahydro-quinoline (33 g, 0.2 mol) in H₂SO₄ (120 mL) was slowly added KNO₃ (20.7 g, 0.2 mol) at 0° C. After addition, the mixture was stirred at room temperature for 2 h, carefully poured into ice water and basified with Na₂CO₃ to pH 8. The mixture was extracted with ethyl acetate (3×200 mL). The combined extracts were washed with water and brine, dried over Na₂SO₄ and concentrated to give 4,4-dimethyl-7-nitro-1,2,3,4-tetrahydro-quinoline (21 g, 50%).

4,4-Dimethyl-7-nitro-3,4-dihydro-2H-quinoline-1-carboxylic acid tert-butyl ester A mixture of 4,4-dimethyl-7-nitro-1,2,3,4-tetrahydro-quinoline (25 g, 0.12 mol) and Boc₂O (55 g, 0.25 mol) was stirred at 80° C. for 2 days. The mixture was purified by silica gel chromatography to give 4,4-dimethyl-7-nitro-3,4-dihydro-2H-quinoline-1-carboxylic acid tert-butyl ester (8 g, 22%).

DC-3; tert-Butyl 7-amino-3,4-dihydro-4,4-dimethylquinoline-1(2H)-carboxylate A mixture of 4,4-dimethyl-7-nitro-3,4-dihydro-2H-quinoline-1 carboxylic acid tert-butyl ester (8.3 g, 0.03 mol) and Pd—C (0.5 g) in methanol (100 mL) was stirred under H₂ (1 atm) at room temperature overnight. The catalyst was filtered off and the filtrate was concentrated. The residue was washed with petroleum ether to give tert-butyl 7-amino-3,4-dihydro-4,4-dimethylquinoline-1(2H)-carboxylate (DC-3) (7.2 g, 95%). ¹H NMR (CDCl₃) δ 7.11-7.04 (m, 2H), 6.45-6.38 (m, 1H), 3.71-3.67 (m, 2H), 3.50-3.28 (m, 2H), 1.71-1.67 (m, 2H), 1.51 (s, 9H), 1.24 (s, 6H).

### Example 4

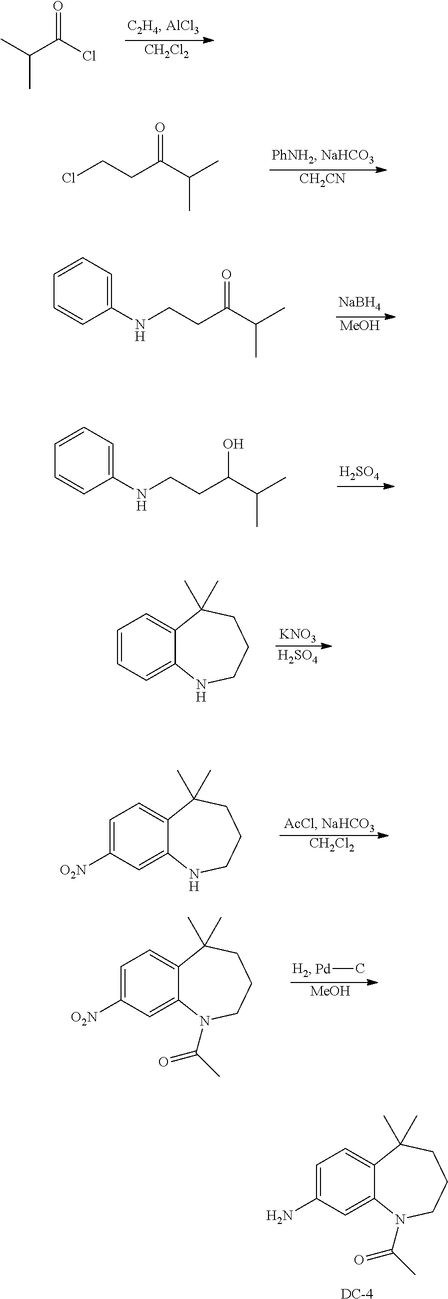

### 1-Chloro-4-methylpentan-3-one

Ethylene was passed through a solution of isobutyryl chloride (50 g, 0.5 mol) and AlCl₃ (68.8 g, 0.52 mol) in anhydrous CH₂Cl₂ (700 mL) at 5° C. After 4 h, the absorption of ethylene ceased, and the mixture was stirred at room temperature overnight. The mixture was poured into cold diluted HCl solution and extracted with CH₂Cl₂. The combined organic phases were washed with brine, dried over Na₂SO₄, filtered and concentrated to give the crude 1-chloro-4-methylpentan-3-one, which was used directly in the next step without further purification.

### 4-Methyl-1-(phenylamino)-pentan-3-one

A suspension of the crude 1-chloro-4-methylpentan-3-one (about 60 g), aniline (69.8 g, 0.75 mol) and NaHCO₃ (210 g, 2.5 mol) in CH₃CN (1000 mL) was heated at reflux overnight. After cooling, the insoluble salt was filtered off and the filtrate was concentrated. The residue was diluted with CH₂Cl₂, washed with 10% HCl solution (100 mL) and brine, dried over Na₂SO₄, filtered and concentrated to give the crude 4-methyl-1-(phenylamino)-pentan-3-one.

### 4-Methyl-1-(phenylamino)-pentan-3-ol

At −10° C., NaBH₄ (56.7 g, 1.5 mol) was gradually added to a mixture of the crude 4-methyl-1-(phenylamino)-pentan-3-one (about 80 g) in MeOH (500 mL). After addition, the reaction mixture was allowed to warm to room temperature and stirred for 20 min. The solvent was removed and the residue was repartitioned between water and CH₂Cl₂. The organic phase was separated, washed with brine, dried over Na₂SO₄, filtered and concentrated. The resulting gum was triturated with ether to give 4-methyl-1-(phenylamino)-pentan-3-ol as a white solid (22 g, 23%).

### 5,5-Dimethyl-2,3,4,5-tetrahydro-1H-benzo[b]azepine

A mixture of 4-methyl-1-(phenylamino)-pentan-3-ol (22 g, 0.11 mol) in 98% H₂SO₄ (250 mL) was stirred at 50° C. for 30 min. The reaction mixture was poured into ice-water basified with sat. NaOH solution to pH 8 and extracted with CH₂Cl₂. The combined organic phases were washed with brine, dried over Na₂SO₄, filtered and concentrated. The residue was purified by column chromatography (petroleum ether) to afford 5,5-dimethyl-2,3,4,5-tetrahydro-1H-benzo[b]azepine as a brown oil (1.5 g, 8%).

### 5,5-Dimethyl-8-nitro-2,3,4,5-tetrahydro-1H-benzo[b]azepine

At 0° C., KNO₃ (0.76 g, 7.54 mmol) was added portionwise to a solution of 5,5-dimethyl-2,3,4,5-tetrahydro-1H-benzo[b]azepine (1.1 g, 6.28 mmol) in H₂SO₄ (15 mL). After stirring 15 min at this temperature, the mixture was poured into ice water, basified with sat. NaHCO₃ to pH 8 and extracted with EtOAc. The organic layer was washed with brine, dried over Na₂SO₄ and concentrated to give crude 5,5-dimethyl-8-nitro-2,3,4,5-tetrahydro-1H-benzo[b]azepine (1.2 g), which was used directly in the next step without further purification.

### 1-(5,5-dimethyl-8-nitro-2,3,4,5-tetrahydrobenzo[b]azepin-1-yl)ethanone

Acetyl chloride (0.77 mL, 11 mmol) was added to a suspension of crude 5,5-dimethyl-8-nitro-2,3,4,5-tetrahydro-1H-benzo[b]azepine (1.2 g, 5.45 mmol) and NaHCO₃ (1.37 g, 16.3 mmol) in CH₂Cl₂ (20 mL). The mixture was heated at reflux for 1 h. After cooling, the mixture was poured into water and extracted with CH₂Cl₂. The organic layer was washed with brine, dried over Na₂SO₄ and concentrated. The residue was purified by column chromatography to afford 1-(5,5-dimethyl-8-nitro-2,3,4,5-tetrahydrobenzo[b]azepin-1-yl)ethanone (1.05 g, 64% over two steps).

### DC-4; 1-(8-Amino-2,3,4,5-tetrahydro-5,5-dimethylbenzo[b]azepin-1-yl)ethanone

A suspension of 1-(5,5-dimethyl-8-nitro-2,3,4,5-tetrahydrobenzo[b]azepin-1-yl)ethanone (1.05 g, 40 mmol) and 10% Pd—C (0.2 g) in MeOH (20 mL) was stirred under H₂ (1 atm) at room temperature for 4 h. After filtration, the filtrate was concentrated to give 1-(8-amino-2,3,4,5-tetrahydro-5,5-dimethylbenzo[b]azepin-1-yl)ethanone as a white solid (DC-4) (880 mg, 94%). ¹H NMR (CDCl₃) δ 7.06 (d, J=8.0 Hz, 1H), 6.59 (dd, J=8.4, 2.4 Hz, 1H), 6.50 (br s, 1H), 4.18-4.05 (m, 1H), 3.46-3.36 (m, 1H), 2.23 (s, 3H), 1.92-1.85 (m, 1H), 1.61-1.51 (m, 3H), 1.21 (s, 3H), 0.73 (t, J=7.2 Hz, 3H); ESI-MS 233.0 m/z (MH⁺).

### Example 5

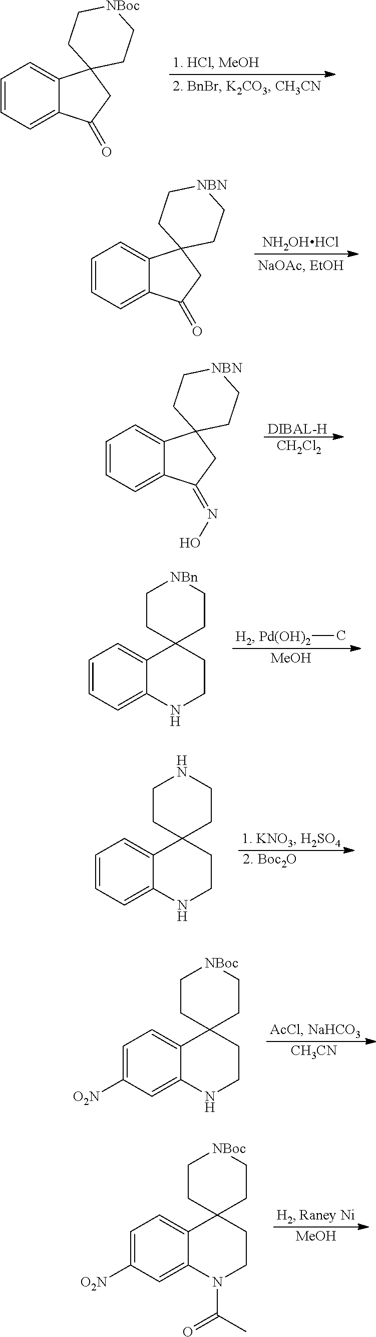

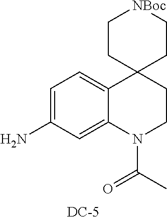 [SMILES: CC(=O)N1CCC2(CCN(C(=O)OC(C)(C)C)CC2)c2ccc(N)cc21 ; "DC-5"]

### Spiro[1H-indene-1,4′-piperidin]-3(2H)-one, 1′-benzyl

A mixture of spiro[1H-indene-1,4′-piperidine]-1′-carboxylic acid, 2,3-dihydro-3-oxo-, 1,1-dimethylethyl ester (9.50 g, 31.50 mmol) in saturated HCl/MeOH (50 mL) was stirred at 25° C. overnight. The solvent was removed under reduced pressure to yield an off-white solid (7.50 g). To a solution of this solid in dry CH₃CN (30 mL) was added anhydrous K₂CO₃ (7.85 g, 56.80 mmol). The suspension was stirred for 5 min, and benzyl bromide (5.93 g, 34.65 mmol) was added dropwise at room temperature. The mixture was stirred for 2 h, poured into cracked ice and extracted with CH₂Cl₂. The combined organic layers were dried over Na₂SO₄ and concentrated under vacuum to give crude spiro[1H-indene-1,4′-piperidin]-3(2H)-one, 1′-benzyl (7.93 g, 87%), which was used without further purification.

### Spiro[1H-indene-1,4′-piperidin]-3(2H)-one, 1′-benzyl, oxime

To a solution of spiro[1H-indene-1,4′-piperidin]-3(2H)-one, 1′-benzyl (7.93 g, 27.25 mmol) in EtOH (50 mL) were added hydroxylamine hydrochloride (3.79 g, 54.50 mmol) and anhydrous sodium acetate (4.02 g, 49.01 mmol) in one portion. The mixture was refluxed for 1 h, and then cooled to room temperature. The solvent was removed under reduced pressure and 200 mL of water was added. The mixture was extracted with CH₂Cl₂. The combined organic layers were dried over Na₂SO₄ and concentrated to yield spiro[1H-indene-1,4′-piperidin]-3(2H)-one, 1′-benzyl, oxime (7.57 g, 91%), which was used without further purification.

### 1,2,3,4-Tetrahydroquinolin-4-spiro-4′-(N′-benzyl-piperidine)

To a solution of spiro[1H-indene-1,4′-piperidin]-3(2H)-one, 1′-benzyl, oxime (7.57 g, 24.74 mmol) in dry CH₂Cl₂ (150 mL) was added dropwise DIBAL-H (135.7 mL, 1M in toluene) at 0° C. The mixture was stirred at 0° C. for 3 h, diluted with CH₂Cl₂ (100 mL), and quenched with NaF (20.78 g, 495 mmol) and water (6.7 g, 372 mmol). The resulting suspension was stirred vigorously at 0° C. for 30 min. After filtration, the residue was washed with CH₂Cl₂. The combined filtrates were concentrated under vacuum to give an off-brown oil that was purified by column chromatography on silica gel (CH₂Cl₂-MeOH, 30:1) to afford 1,2,3,4-tetrahydroquinolin-4-spiro-4′-(N′-benzyl-piperidine) (2.72 g, 38%).

### 1,2,3,4-Tetrahydroquinolin-4-spiro-4′-piperidine

A suspension of 1,2,3,4-Tetrahydroquinolin-4-spiro-4′-(N′-benzyl-piperidine) (300 mg, 1.03 mmol) and Pd(OH)₂—C (30 mg) in MeOH (3 mL) was stirred under H₂ (55 psi) at 50° C. over night. After cooling, the catalyst was filtered off and washed with MeOH. The combined filtrates were concentrated under reduced pressure to yield 1,2,3,4-tetrahydroquinolin-4-spiro-4′-piperidine as a white solid (176 mg, 85%), which was used without further purification.

### 7′-Nitro-spiro[piperidine-4,4′(1H)-quinoline], 2′,3′-dihydro-carboxylic acid tert-butyl ester

KNO₃ (69.97 mg, 0.69 mmol) was added portion-wise to a suspension of 1,2,3,4-tetrahydroquinolin-4-spiro-4′-piperidine (133 mg, 0.66 mmol) in 98% H₂SO₄ (2 mL) at 0° C. After the addition was complete, the reaction mixture was allowed to warm to room temperature and stirred for additional 2 h. The mixture was then poured into cracked ice and basified with 10% NaOH to pH ˜8. Boc₂O (172 mg, 0.79 mmol) was added dropwise and the mixture was stirred at room temperature for 1 h. The mixture was then extracted with EtOAc and the combined organic layers were dried over Na₂SO₄, filtered and concentrated to yield crude T-nitro-spiro[piperidine-4,4′(1′H)-quinoline], 2′,3′-dihydro-carboxylic acid tert-butyl ester (230 mg), which was used in the next step without further purification.

### 7′-nitro-spiro[piperidine-4,4′(1′H)-1-acetyl-quinoline], 2′,3′-dihydro-carboxylic acid tert-butyl ester

Acetyl chloride (260 mg, 3.30 mmol) was added dropwise to a suspension of 7′-nitro-spiro[piperidine-4,4′(1′H)-quinoline], 2′,3′-dihydro-carboxylic acid tert-butyl ester (230 mg) and NaHCO₃ (1.11 g, 13.17 mmol) in MeCN (5 mL) at room temperature. The reaction mixture was refluxed for 4 h. After cooling, the suspension was filtered and the filtrate was concentrated. The residue was purified by column chromatography (petroleum ether-EtOAc, 10:1) to provide 7′-nitro-spiro[piperidine-4,4′(1′H)-1-acetyl-quinoline], 2′,3′-dihydro-carboxylic acid tert-butyl ester (150 mg, 58% over 2 steps)

### DC-5; 7′Amino-spiro[piperidine-4,4′(1′H)-1-acetyl-quinoline], 2′,3′-dihydro-carboxylic acid tert-butyl ester

A suspension of 7′-nitro-spiro[piperidine-4,4′(1′H)-1-acetyl-quinoline], 2′,3′-dihydro-carboxylic acid tert-butyl ester (150 mg, 0.39 mmol) and Raney Ni (15 mg) in MeOH (2 mL) was stirred under H₂ (1 atm) at 25° C. overnight. The catalyst was removed via filtration and washed with MeOH. The combined filtrates were dried over Na₂SO₄, filtered, and concentrated to yield 7′-amino-spiro[piperidine-4,4′(1′H)-1-acetyl-quinoline], 2′,3′-dihydro-carboxylic acid tert-butyl ester (DC-5) (133 mg, 96%).

### Example 7

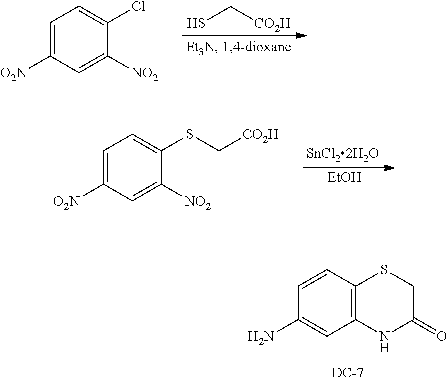

### 2-(2,4-Dinitrophenylthio)-acetic acid

Et₃N (1.5 g, 15 mmol) and mercapto-acetic acid (1 g, 11 mmol) were added to a solution of 1-chloro-2,4-dinitrobenzene (2.26 g, 10 mmol) in 1,4-dioxane (50 mL) at room temperature. After stirring at room temperature for 5 h, H₂O (100 mL) was added. The resulting suspension was extracted with ethyl acetate (100 mL×3). The ethyl acetate extract was washed with water and brine, dried over Na₂SO₄ and concentrated to give 2-(2,4-dinitrophenylthio)-acetic acid (2.3 g, 74%), which was used without further purification.

### DC-7; 6-Amino-2H-benzo[b][1,4]thiazin-3(4H)-one

A solution of 2-(2,4-dinitrophenylthio)-acetic acid (2.3 g, 9 mmol) and tin (II) chloride dihydrate (22.6 g, 0.1 mol) in ethanol (30 mL) was refluxed overnight. After removal of the solvent under reduced pressure, the residual slurry was diluted with water (100 mL) and basified with 10% Na₂CO₃ solution to pH 8. The resulting suspension was extracted with ethyl acetate (3×100 mL). The ethyl acetate extract was washed with water and brine, dried over Na₂SO₄, and concentrated. The residue was washed with CH₂Cl₂ to yield 6-amino-2H-benzo[b][1,4]thiazin-3(4H)-one (DC-7) as a yellow powder (1 g, 52%). ¹H NMR (DMSO-d₆) δ 10.24 (s. 1H), 6.88 (d, 1H, J=6 Hz), 6.19-6.21 (m, 2H), 5.15 (s, 2H), 3.28 (s, 2H); ESI-MS 181.1 m/z (MH⁺).

### Example 7

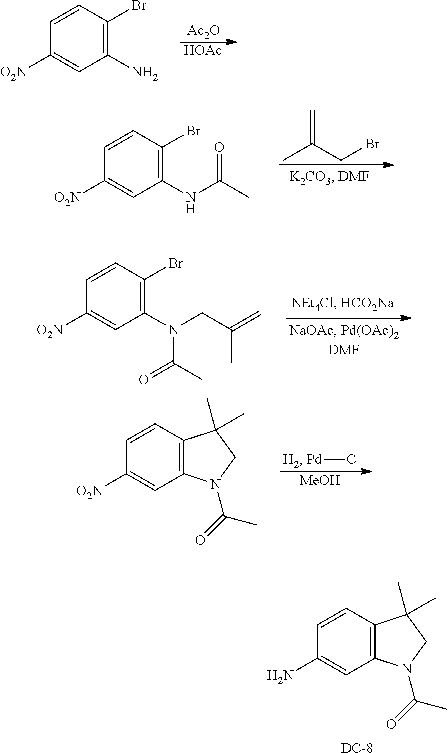

### N-(2-Bromo-5-nitrophenyl)acetamide

Acetic anhydride (1.4 mL, 13.8 mmol) was added dropwise to a stirring solution of 2-bromo-5-nitroaniline (3 g, 13.8 mmol) in glacial acetic acid (30 mL) at 25° C. The reaction mixture was stirred at room temperature overnight, and then poured into water. The precipitate was collected via filtration, washed with water and dried under vacuum to provide N-(2-bromo-5-nitrophenyl)acetamide as an off white solid (3.6 g, 90%).

### N-(2-Bromo-5-nitrophenyl)-N-(2-methylprop-2-enyl)acetamide

At 25° C., a solution of 3-bromo-2-methylpropene (3.4 g, 55.6 mmol) in anhydrous DMF (30 mL) was added dropwise to a solution of N-(2-bromo-5-nitrophenyl)acetamide (3.6 g, 13.9 mmol) and potassium carbonate (3.9 g, 27.8 mmol) in anhydrous DMF (50 mL). The reaction mixture was stirred at 25° C. overnight. The reaction mixture was then filtered and the filtrate was treated with sat. Na₂CO₃ solution. The organic layer was separated and the aqueous layer was extracted with EtOAc. The combined organic extracts were washed with water and brine, dried over MgSO₄, filtered and concentrated under vacuum to provide N-(2-bromo-5-nitrophenyl)-N-(2-methylprop-2-enyl)acetamide as a golden solid (3.1 g, 85%). ESI-MS 313 m/z (MH⁺).

### 1-(3,3-Dimethyl-6-nitroindolin-1-yl)ethanone

A solution of N-(2-bromo-5-nitrophenyl)-N-(2-methylprop-2-enyl)acetamide (3.1 g, 10.2 mmol), tetraethylammonium chloride hydrate (2.4 g, 149 mmol), sodium formate (1.08 g, 18 mmol), sodium acetate (2.76 g, 34.2 mmol) and palladium acetate (0.32 g, 13.2 mmol) in anhydrous DMF (50 mL) was stirred at 80° C. for 15 h under N₂ atmosphere. After cooling, the mixture was filtered through Celite. The Celite was washed with EtOAc and the combined filtrates were washed with sat. NaHCO₃. The separated organic layer was washed with water and brine, dried over MgSO₄, filtered and concentrated under reduced pressure to provide 1-(3,3-dimethyl-6-nitroindolin-1-yl)ethanone as a brown solid (2.1 g, 88%).

### DC-8; 1-(6-Amino-3,3-dimethyl-2,3-dihydro-indol-1-yl)-ethanone

10% Pd—C (0.2 g) was added to a suspension of 1-(3,3-dimethyl-6-nitroindolin-1-yl)ethanone (2.1 g, 9 mmol) in MeOH (20 mL). The reaction was stirred under H₂ (40 psi) at room temperature overnight. Pd—C was filtered off and the filtrate was concentrated under vacuum to give a crude product, which was purified by column chromatography to yield 1-(6-amino-3,3-dimethyl-2,3-dihydro-indol-1-yl)-ethanone (DC-8) (1.3 g, 61%).

### Example 8

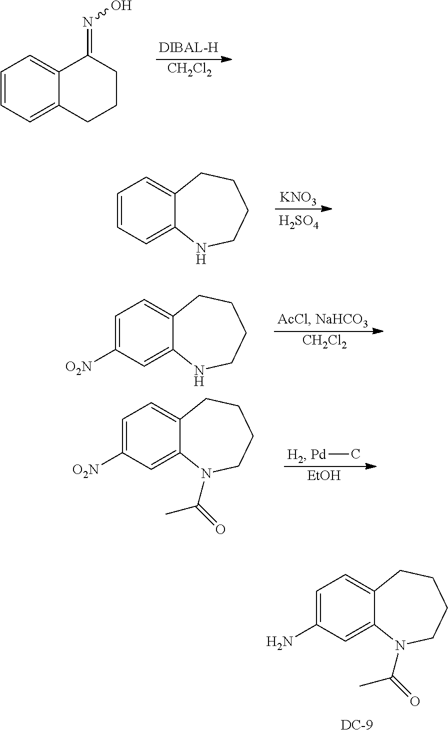

### 2,3,4,5-Tetrahydro-1H-benzo[b]azepine

DIBAL (90 mL, 90 mmol) was added dropwise to a solution of 4-dihydro-2H-naphthalen-1-one oxime (3 g, 18 mmol) in dichloromethane (50 mL) at 0° C. The mixture was stirred at this temperature for 2 h. The reaction was quenched with dichloromethane (30 mL), followed by treatment with NaF (2 g. 0.36 mol) and H₂O (5 mL, 0.27 mol). Vigorous stirring of the resulting suspension was continued at 0° C. for 30 min. After filtration, the filtrate was concentrated. The residue was purified by flash column chromatography to give 2,3,4,5-tetrahydro-1H-benzo[b]azepine as a colorless oil (1.9 g, 70%).

### 8-Nitro-2,3,4,5-tetrahydro-1H-benzo[b]azepine

At −10° C., 2,3,4,5-tetrahydro-1H-benzo[b]azepine (1.9 g, 13 mmol) was added dropwise to a solution of KNO₃ (3 g, 30 mmol) in H₂SO₄ (50 mL). The mixture was stirred for 40 min, poured over crushed ice, basified with aq. ammonia to pH 13, and extracted with EtOAc. The combined organic phases were washed with brine, dried over Na₂SO₄ and concentrated to give 8-nitro-2,3,4,5-tetrahydro-1H-benzo[b]azepine as a black solid (1.3 g, 51%), which was used without further purification.

### 1-(8-Nitro-2,3,4,5-tetrahydro-benzo[b]azepin-1-yl)-ethanone

Acetyl chloride (1 g, 13 mmol) was added dropwise to a mixture of 8-nitro-2,3,4,5-tetrahydro-1H-benzo[b]azepine (1.3 g, 6.8 mmol) and NaHCO₃ (1 g, 12 mmol) in CH₂Cl₂ (50 mL). After stirring for 1 h, the mixture was filtered and the filtrate was concentrated. The residue was dissolved in CH₂Cl₂, washed with brine, dried over Na₂SO₄ and concentrated. The residue was purified by column chromatography to give 1-(8-nitro-2,3,4,5-tetrahydro-benzo[b]azepin-1-yl)-ethanone as a yellow solid (1.3 g, 80%).

### DC-9; 1-(8-Amino-2,3,4,5-tetrahydro-benzo[b]azepin-1-yl)-ethanone

A mixture of 1-(8-nitro-2,3,4,5-tetrahydro-benzo[b]azepin-1-yl)-ethanone (1.3 g, 5.4 mmol) and Pd—C (10%, 100 mg) in EtOH (200 mL) was stirred under H₂ (1 atm) at room temperature for 1.5 h. The mixture was filtered through a layer of Celite and the filtrate was concentrated to give 1-(8-amino-2,3,4,5-tetrahydro-benzo[b]azepin-1-yl)-ethanone (DC-9) as a white solid (1 g, 90%). ¹H NMR (CDCl₃) δ 7.01 (d, J=6.0 Hz, 1H), 6.56 (dd, J=6.0, 1.8 Hz, 1H), 6.50 (d, J=1.8 Hz, 1H), 4.66-4.61 (m, 1H), 3.50 (br s, 2H), 2.64-2.55 (m, 3H), 1.94-1.91 (m, 5H), 1.77-1.72 (m, 1H), 1.32-1.30 (m, 1H); ESI-MS 204.1 m/z (MH⁺).

### Example 9

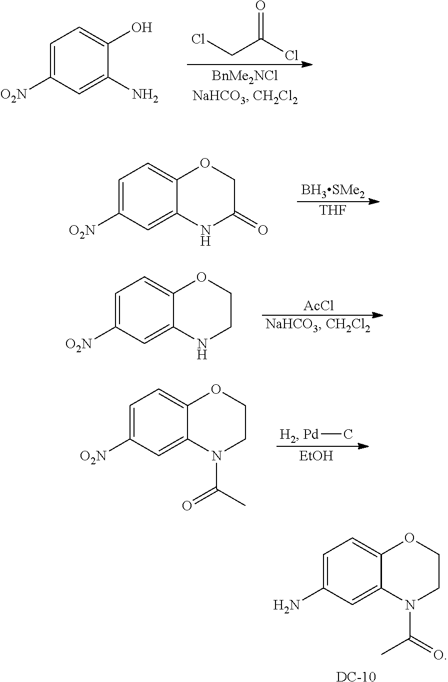

### 6-Nitro-4H-benzo[1,4]oxazin-3-one

At 0° C., chloroacetyl chloride (8.75 mL, 0.11 mol) was added dropwise to a mixture of 4-nitro-2-aminophenol (15.4 g, 0.1 mol), benzyltrimethylammonium chloride (18.6 g, 0.1 mol) and NaHCO₃ (42 g, 0.5 mol) in chloroform (350 ml) over a period of 30 min. After addition, the reaction mixture was stirred at 0° C. for 1 h, then at 50° C. overnight. The solvent was removed under reduced pressure and the residue was treated with water (50 ml). The solid was collected via filtration, washed with water and recrystallized from ethanol to provide 6-nitro-4H-benzo[1,4]oxazin-3-one as a pale yellow solid (8 g, 41%).

### 6-Nitro-3,4-dihydro-2H-benzo[1,4]oxazine

A solution of BH₃.Me₂S in THF (2 M, 7.75 mL, 15.5 mmol) was added dropwise to a suspension of 6-nitro-4H-benzo[1,4]oxazin-3-one (0.6 g, 3.1 mmol) in THF (10 mL). The mixture was stirred at room temperature overnight. The reaction was quenched with MeOH (5 mL) at 0° C. and then water (20 mL) was added. The mixture was extracted with Et₂O and the combined organic layers were washed with brine, dried over Na₂SO₄ and concentrated to give 6-nitro-3,4-dihydro-2H-benzo[1,4]oxazine as a red solid (0.5 g, 89%), which was used without further purification.

### 4-Acetyl-6-nitro-3,4-dihydro-2H-benzo[1,4]oxazine

Under vigorous stirring at room temperature, acetyl chloride (1.02 g, 13 mmol) was added dropwise to a mixture of 6-nitro-3,4-dihydro-2H-benzo[1,4]oxazine (1.8 g, 10 mmol) and NaHCO₃ (7.14 g, 85 mmol) in CH₂Cl₂ (50 mL). After addition, the reaction was stirred for 1 h at this temperature. The mixture was filtered and the filtrate was concentrated under vacuum. The residue was treated with Et₂O: hexane (1:2, 50 mL) under stirring for 30 min and then filtered to give 4-acetyl-6-nitro-3,4-dihydro-2H-benzo[1,4]oxazine as a pale yellow solid (2 g, 90%).

### DC-10; 4-Acetyl-6-amino-3,4-dihydro-2H-benzo[1,4]oxazine

A mixture of 4-acetyl-6-nitro-3,4-dihydro-2H-benzo[1,4]oxazine (1.5 g, 67.6 mmol) and Pd—C (10%, 100 mg) in EtOH (30 mL) was stirred under H₂ (1 atm) overnight. The catalyst was filtered off and the filtrate was concentrated. The residue was treated with HCl/MeOH to give 4-acetyl-6-amino-3,4-dihydro-2H-benzo[1,4]oxazine hydrochloride (DC-10) as an off-white solid (1.1 g, 85%). ¹H NMR (DMSO-d₆) δ 10.12 (br s, 2H), 8.08 (br s, 1H), 6.90-7.03 (m, 2H), 4.24 (t, J=4.8 Hz, 2H), 3.83 (t, J=4.8 Hz, 2H), 2.23 (s, 3H); ESI-MS 192.1 m/z (MH⁺).

### Example 10

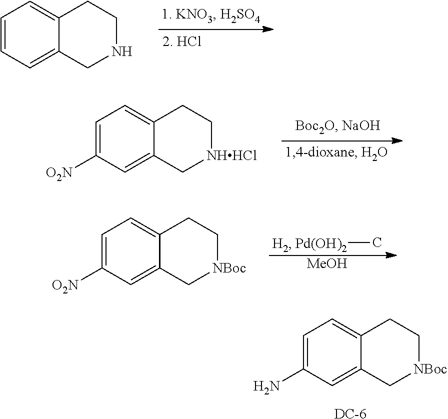

### 1,2,3,4-Tetrahydro-7-nitroisoquinoline hydrochloride

1,2,3,4-Tetrahydroisoquinoline (6.3 mL, 50.0 mmol) was added dropwise to a stirred ice-cold solution of concentrated H₂SO₄ (25 mL). KNO₃ (5.6 g, 55.0 mmol) was added portionwise while maintaining the temperature below 5° C. The mixture was stirred at room temperature overnight, carefully poured into an ice-cold solution of concentrated NH₄OH, and then extracted three times with CHCl₃. The combined organic layers were washed with brine, dried over Na₂SO₄ and concentrated. The resulting dark brown oil was taken up into EtOH, cooled in an ice bath and treated with concentrated HCl. The yellow precipitate was collected via filtration and recrystallized from methanol to give 1,2,3,4-tetrahydro-7-nitroisoquinoline hydrochloride as yellow solid (2.5 g, 23%). ¹H NMR (400 MHz, DMSO-d6) δ 9.86 (s, 2H), 8.22 (d, J=1.6 Hz, 1H), 8.11 (dd, J=8.5, 2.2 Hz, 1H), 7.53 (d, J=8.5 Hz, 1H), 4.38 (s, 2H), 3.38 (s, 2H), 3.17-3.14 (m, 2H); HPLC ret. time 0.51 min, 10-99% CH₃CN, 5 min run; ESI-MS 179.0 m/z (MH⁺).

### tert-Butyl 3,4-dihydro-7-nitroisoquinoline-2(1H)-carboxylate

A mixture of 1,2,3,4-Tetrahydro-7-nitroisoquinoline (2.5 g, 11.6 mmol), 1,4-dioxane (24 mL), H₂O (12 mL) and 1N NaOH (12 mL) was cooled in an ice-bath, and Boc₂O (2.8 g, 12.8 mmol) was added. The mixture was stirred at room temperature for 2.5 h, acidified with a 5% KHSO₄ solution to pH 2-3, and then extracted with EtOAc. The organic layer was dried over MgSO₄ and concentrated to give tert-butyl 3,4-dihydro-7-nitroisoquinoline-2(1H)-carboxylate (3.3 g, quant.), which was used without further purification. ¹H NMR (400 MHz, DMSO-d6) δ 8.13 (d, J=2.3 Hz, 1H), 8.03 (dd, J=8.4, 2.5 Hz, 1H), 7.45 (d, J=8.5 Hz, 1H), 4.63 (s, 2H), 3.60-3.57 (m, 2H), 2.90 (t, J=5.9 Hz, 2H), 1.44 (s, 9H); HPLC ret. time 3.51 min, 10-99% CH₃CN, 5 min run; ESI-MS 279.2 m/z (MH⁺).

### DC-6; tert-Butyl 7-amino-3,4-dihydroisoquinoline-2(1H)-carboxylate

Pd(OH)₂ (330.0 mg) was added to a stirring solution of tert-butyl 3,4-dihydro-7-nitroisoquinoline-2(1H)-carboxylate (3.3 g, 12.0 mmol) in MeOH (56 mL) under N₂ atmosphere. The reaction mixture was stirred under H₂ (1 atm) at room temperature for 72 h. The solid was removed by filtration through Celite. The filtrate was concentrated and purified by column chromatography (15-35% EtOAc-Hexanes) to provide tert-butyl 7-amino-3,4-dihydroisoquinoline-2(1H)-carboxylate (DC-6) as a pink oil (2.0 g, 69%). ¹H NMR (400 MHz, DMSO-d6) δ 6.79 (d, J=8.1 Hz, 1H), 6.40 (dd, J=8.1, 2.3 Hz, 1H), 6.31 (s, 1H), 4.88 (s, 2H), 4.33 (s, 2H), 3.48 (t, J=5.9 Hz, 2H), 2.58 (t, J=5.9 Hz, 2H), 1.42 (s, 9H); HPLC ret. time 2.13 min, 10-99% CH₃CN, 5 min run; ESI-MS 249.0 m/z (MH⁺).

### Other Amines

### Example 1

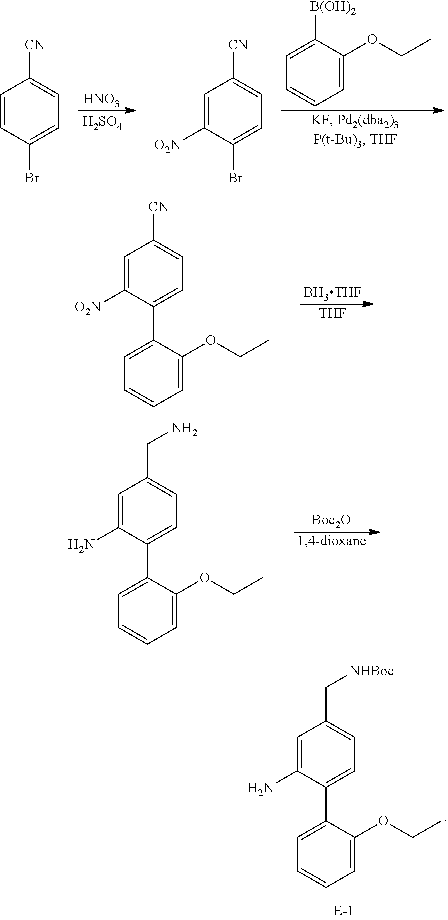

### 4-Bromo-3-nitrobenzonitrile

To a solution of 4-bromobenzonitrile (4.0 g, 22 mmol) in conc. H₂SO₄ (10 mL) was added dropwise at 0° C. nitric acid (6 mL). The reaction mixture was stirred at 0° C. for 30 min, and then at room temperature for 2.5 h. The resulting solution was poured into ice-water. The white precipitate was collected via filtration and washed with water until the washings were neutral. The solid was recrystallized from an ethanol/water mixture (1:1, 20 mL) twice to afford 4-bromo-3-nitrobenzonitrile as a white crystalline solid (2.8 g, 56%). ¹H NMR (300 MHz, DMSO-d₆) δ 8.54 (s, 1H), 8.06 (d, J=8.4 Hz, 1H), 7.99 (d, J=8.4 Hz, 1H); ¹³C NMR (75 MHz, DMSO-d₆) δ 150.4, 137.4, 136.6, 129.6, 119.6, 117.0, 112.6; HPLC ret. time 1.96 min, 10-100% CH₃CN, 5 min gradient; ESI-MS 227.1 m/z (MH⁺).

### 2′-Ethoxy-2-nitrobiphenyl-4-carbonitrile

A 50 mL round-bottom flask was charged with 4-bromo-3-nitrobenzonitrile (1.0 g 4.4 mmol), 2-ethoxyphenylboronic acid (731 mg, 4.4 mmol), Pd₂(dba)₃ (18 mg, 0.022 mmol) and potassium fluoride (786 mg, 13.5 mmol). The reaction vessel was evacuated and filled with argon. Dry THF (300 mL) was added followed by the addition of P(t-Bu)₃ (0.11 mL, 10% wt. in hexane). The reaction mixture was stirred at room temperature for 30 min., and then heated at 80° C. for 16 h. After cooling to room temperature, the resulting mixture was filtered through a Celite pad and concentrated. 2′-Ethoxy-2-nitrobiphenyl-4-carbonitrile was isolated as a yellow solid (1.12 g, 95%). ¹H NMR (300 MHz, DMSO-d₆) δ 8.51 (s, 1H), 8.20 (d, J=8.1 Hz, 1H), 7.68 (d, J=8.4 Hz, 1H), 7.41 (t, J=8.4 Hz, 1H), 7.37 (d, J=7.5 Hz, 1H), 7.08 (t, J=7.5 Hz, 1H), 7.03 (d, J=8.1 Hz, 1H), 3.91 (q, J=7.2 Hz, 2H), 1.12 (t, J=7.2 Hz, 3H); ¹³C NMR (75 MHz, DMSO-d₆) δ 154.9, 149.7, 137.3, 137.2, 134.4, 131.5, 130.4, 128.4, 125.4, 121.8, 117.6, 112.3, 111.9, 64.1, 14.7; HPLC ret. time 2.43 min, 10-100% CH₃CN, 5 min gradient; ESI-MS 269.3 m/z (MH⁺).

### 4-Aminomethyl-2′-ethoxy-biphenyl-2-ylamine

To a solution of 2′-ethoxy-2-nitrobiphenyl-4-carbonitrile (500 mg, 1.86 mmol) in THF (80 mL) was added a solution of BH₃.THF (5.6 mL, 10% wt. in THF, 5.6 mmol) at 0° C. over 30 min. The reaction mixture was stirred at 0° C. for 3 h and then at room temperature for 15 h. The reaction solution was chilled to 0° C., and a H₂O/THF mixture (3 mL) was added. After being agitated at room temperature for 6 h, the volatiles were removed under reduced pressure. The residue was dissolved in EtOAc (100 mL) and extracted with 1N HCl (2×100 mL). The aqueous phase was basified with 1N NaOH solution to pH 1 and extracted with EtOAc (3×50 mL). The combined organic layers were washed with water (50 mL), dried over Na₂SO₄, filtered, and evaporated. After drying under vacuum, 4-aminomethyl-2′-ethoxy-biphenyl-2-ylamine was isolated as a brown oil (370 mg, 82%). ¹H NMR (300 MHz, DMSO-d₆) δ 7.28 (dt, J=7.2 Hz, J=1.8 Hz, 1H), 7.09 (dd, J=7.2 Hz, J=1.8 Hz, 1H), 7.05 (d, J=7.5 Hz, 1H), 6.96 (dt, J=7.2 Hz, J=0.9 Hz, 1H), 6.83 (d, J=7.5 Hz, 1H), 6.66 (d, J=1.2 Hz, 1H), 6.57 (dd, J=7.5 Hz, J=1.5 Hz, 1H), 4.29 (s, 2H), 4.02 (q, J=6.9 Hz, 2H), 3.60 (s, 2H), 1.21 (t, J=6.9 Hz, 3H); HPLC ret. time 1.54 min, 10-100% CH₃CN, 5 min gradient; ESI-MS 243.3 m/z (MH⁺).

### E-1; (2-Amino-2′-ethoxy-biphenyl-4-ylmethyl)carbamic acid tert-butyl ester

A solution of Boc₂O (123 mg, 0.565 mmol) in 1,4-dioxane (10 mL) was added over a period of 30 min. to a solution of 4-aminomethyl-2′-ethoxy-biphenyl-2-ylamine (274 mg, 1.13 mmol) in 1,4-dioxane (10 mL). The reaction mixture was stirred at room temperature for 16 h. The volatiles were removed on a rotary evaporator. The residue was purified by flash chromatography (silica gel, EtOAc—CH₂Cl₂, 1:4) to afford (2-Amino-2′-ethoxy-biphenyl-4-ylmethyl)carbamic acid tert-butyl ester (E-1) as a pale yellow oil (119 mg, 31%). ¹H NMR (300 MHz, DMSO-d₆) δ 7.27 (m, 2H), 7.07 (dd, J=7.2 Hz, J=1.8 Hz, 1H), 7.03 (d, J=7.8 Hz, 1H), 6.95 (dt, J=7.2 Hz, J=0.9 Hz, 1H), 6.81 (d, J=7.5 Hz, 1H), 6.55 (s, 1H), 6.45 (dd, J=7.8 Hz, J=1.5 Hz, 1H), 4.47 (s, 2H), 4.00 (q, J=7.2 Hz, 2H), 1.38 (s, 9H), 1.20 (t, J=7.2 Hz, 3H); HPLC ret. time 2.34 min, 10-100% CH₃CN, 5 min gradient; ESI-MS 343.1 m/z (MH⁺).

### Example 2

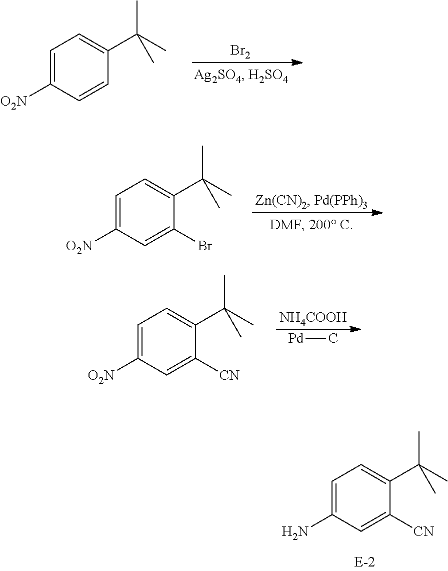

### 2-Bromo-1-tert-butyl-4-nitrobenzene

To a solution of 1-tert-butyl-4-nitrobenzene (8.95 g, 50 mmol) and silver sulfate (10 g, 32 mmol) in 50 mL of 90% sulfuric acid was added dropwise bromine (7.95 g, 50 mmol). Stirring was continued at room temperature overnight, and then the mixture was poured into dilute sodium hydrogen sulfite solution and was extracted with EtOAc three times. The combined organic layers were washed with brine and dried over MgSO₄. After filtration, the filtrate was concentrated to give 2-bromo-1-tert-butyl-4-nitrobenzene (12.7 g, 98%), which was used without further purification. ¹H NMR (400 MHz, CDCl₃) δ 8.47 (d, J=2.5 Hz, 1H), 8.11 (dd, J=8.8, 2.5 Hz, 1H), 7.63 (d, J=8.8 Hz, 1H), 1.57 (s, 9H); HPLC ret. time 4.05 min, 10-100% CH₃CN, 5 min gradient.

### 2-tert-Butyl-5-nitrobenzonitrile

To a solution of 2-bromo-1-tert-butyl-4-nitrobenzene (2.13 g, 8.2 mmol) and Zn(CN)₂ (770 mg, 6.56 mmol) in DMF (10 mL) was added Pd(PPh₃)₄ (474 mg, 0.41 mmol) under a nitrogen atmosphere. The mixture was heated in a sealed vessel at 205° C. for 5 h. After cooling to room temperature, the mixture was diluted with water and extracted with EtOAc twice. The combined organic layers were washed with brine and dried over MgSO₄. After removal of solvent, the residue was purified by column chromatography (0-10% EtOAc-Hexane) to give 2-tert-butyl-5-nitrobenzonitrile (1.33 g, 80%). ¹H NMR (400 MHz, CDCl₃) δ 8.55 (d, J=2.3 Hz, 1H), 8.36 (dd, J=8.8, 2.2 Hz, 1H), 7.73 (d, J=8.9 Hz, 1H), 1.60 (s, 9H); HPLC ret. time 3.42 min, 10-100% CH₃CN, 5 min gradient.

### E-2; 2-tert-Butyl-5-aminobenzonitrile

To a refluxing solution of 2-tert-butyl-5-nitrobenzonitrile (816 mg, 4.0 mmol) in EtOH (20 mL) was added ammonium formate (816 mg, 12.6 mmol), followed by 10% Pd—C (570 mg). The reaction mixture was refluxed for additional 90 min, cooled to room temperature and filtered through Celite. The filtrate was concentrated to give 2-tert-butyl-5-aminobenzonitrile (E-2) (630 mg, 91%), which was used without further purification. HPLC ret. time 2.66 min, 10-99% CH₃CN, 5 min run; ESI-MS 175.2 m/z (MH⁺).

### Example 3

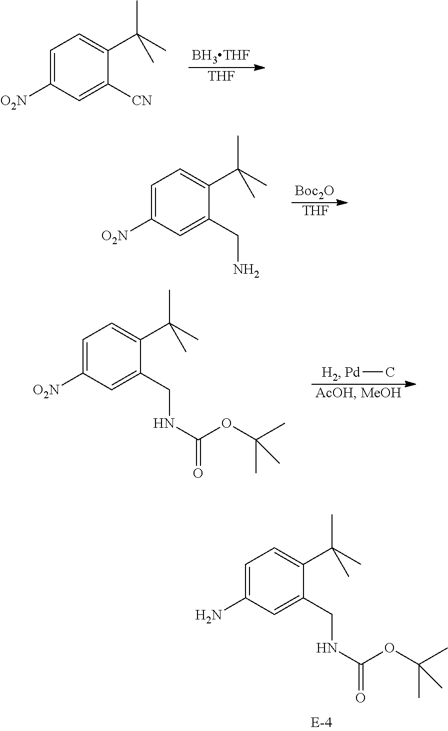

### (2-tert-Butyl-5-nitrophenyl)methanamine

To a solution of 2-tert-butyl-5-nitrobenzonitrile (612 mg, 3.0 mmol) in THF (10 mL) was added a solution of BH₃.THF (12 mL, 1M in THF, 12.0 mmol) under nitrogen. The reaction mixture was stirred at 70° C. overnight and cooled to 0° C. Methanol (2 mL) was added followed by the addition of 1N HCl (2 mL). After refluxing for 30 min, the solution was diluted with water and extracted with EtOAc. The aqueous layer was basified with 1N NaOH and extracted with EtOAc twice. The combined organic layers were washed with brine and dried over Mg₂SO₄. After removal of solvent, the residue was purified by column chromatography (0-10% MeOH—CH₂Cl₂) to give (2-tert-butyl-5-nitrophenyl)methanamine (268 mg, 43%). ¹H NMR (400 MHz, DMSO-d₆) δ 8.54 (d, J=2.7 Hz, 1H), 7.99 (dd, J=8.8, 2.8 Hz, 1H), 7.58 (d, J=8.8 Hz, 1H), 4.03 (s, 2H), 2.00 (t, J=2.1 Hz, 2H), 1.40 (s, 9H); HPLC ret. time 2.05 min, 10-100% CH₃CN, 5 min gradient; ESI-MS 209.3 m/z (MH⁺).

### tert-Butyl 2-tert-butyl-5-nitrobenzylcarbamate

A solution of (2-tert-butyl-5-nitrophenyl)methanamine (208 mg, 1 mmol) and Boc₂O (229 mg, 1.05 mmol) in THF (5 mL) was refluxed for 30 min. After cooling to room temperature, the solution was diluted with water and extracted with EtOAc. The combined organic layers were washed with brine and dried over MgSO₄. After filtration, the filtrate was concentrated to give tert-butyl 2-tert-butyl-5-nitrobenzylcarbamate (240 mg, 78%), which was used without further purification. ¹H NMR (400 MHz, DMSO-d₆) δ 8.26 (d, J=2.3 Hz, 1H), 8.09 (dd, J=8.8, 2.5 Hz, 1H), 7.79 (t, J=5.9 Hz, 1H), 7.68 (d, J=8.8 Hz, 1H), 4.52 (d, J=6.0 Hz, 2H), 1.48 (s, 18H); HPLC ret. time 3.72 min, 10-100% CH₃CN, 5 min gradient.

### E-4; tert-Butyl 2-tert-butyl-5-aminobenzylcarbamate

To a solution of tert-butyl 2-tert-butyl-5-nitrobenzylcarbamate (20 mg, 0.065 mmol) in 5% AcOH-MeOH (1 mL) was added 10% Pd—C (14 mg) under nitrogen atmosphere. The mixture was stirred under H₂ (1 atm) at room temperature for 1 h. The catalyst was removed via filtration through Celite, and the filtrate was concentrated to give tert-butyl 2-tert-butyl-5-aminobenzylcarbamate (E-4), which was used without further purification. ¹H NMR (400 MHz, CDCl₃) δ 7.09 (d, J=8.5 Hz, 1H), 6.62 (d, J=2.6 Hz, 1H), 6.47 (dd, J=8.5, 2.6 Hz, 1H), 4.61 (br s, 1H), 4.40 (d, J=5.1 Hz, 2H), 4.15 (br s, 2H), 1.39 (s, 9H), 1.29 (s, 9H); HPLC ret. time 2.47 min, 10-100% CH₃CN, 5 min gradient; ESI-MS 279.3 m/z (MH⁺).

### Example 4

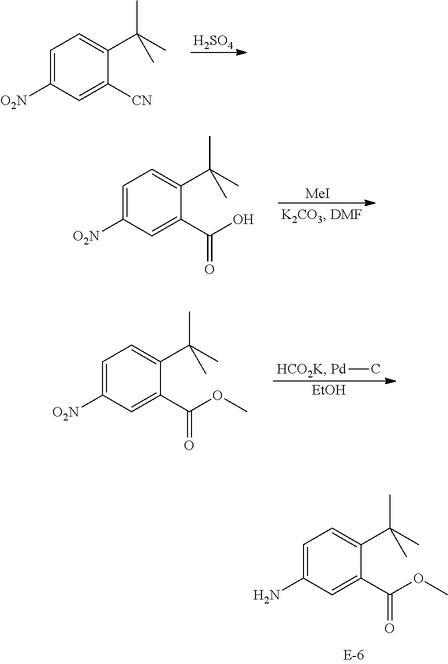

### 2-tert-Butyl-5-nitrobenzoic acid

A solution of 2-tert-butyl-5-nitrobenzonitrile (204 mg, 1 mmol) in 5 mL of 75% H₂SO₄ was microwaved at 200° C. for 30 min. The reaction mixture was poured into ice, extracted with EtOAc, washed with brine and dried over MgSO₄. After filtration, the filtrate was concentrated to give 2-tert-butyl-5-nitrobenzoic acid (200 mg, 90%), which was used without further purification. ¹H NMR (400 MHz, CDCl₃) δ 8.36 (d, J=2.6 Hz, 1H), 8.24 (dd, J=8.9, 2.6 Hz, 1H), 7.72 (d, J=8.9 Hz, 1H) 1.51 (s, 9H); HPLC ret. time 2.97 min, 10-100% CH₃CN, 5 min gradient.

### Methyl 2-tert-butyl-5-nitrobenzoate

To a mixture of 2-tert-butyl-5-nitrobenzoic acid (120 mg, 0.53 mmol) and K₂CO₃ (147 mg, 1.1 mmol) in DMF (5.0 mL) was added CH₃I (40 μL, 0.64 mmol). The reaction mixture was stirred at room temperature for 10 min, diluted with water and extracted with EtOAc. The combined organic layers were washed with brine and dried over MgSO₄. After filtration, the filtrate was concentrated to give methyl 2-tert-butyl-5-nitrobenzoate, which was used without further purification. ¹H NMR (400 MHz, CDCl₃) δ 8.20 (d, J=2.6 Hz, 1H), 8.17 (t, J=1.8 Hz, 1H), 7.66 (d, J=8.6 Hz, 1H), 4.11 (s, 3H), 1.43 (s, 9H).

### E-6; Methyl 2-tert-butyl-5-aminobenzoate

To a refluxing solution of 2-tert-butyl-5-nitrobenzoate (90 mg, 0.38 mmol) in EtOH (2.0 mL) was added potassium formate (400 mg, 4.76 mmol) in water (1 mL), followed by the addition of 20 mg of 10% Pd—C. The reaction mixture was refluxed for additional 40 min, cooled to room temperature and filtered through Celite. The filtrate was concentrated to give methyl 2-tert-butyl-5-aminobenzoate (E-6) (76 mg, 95%), which was used without further purification. ¹H NMR (400 MHz, CDCl₃) δ 7.24 (d, J=8.6 Hz, 1H), 6.67 (dd, J=8.6, 2.7 Hz, 1H), 6.60 (d, J=2.7 Hz, 1H), 3.86 (s, 3H), 1.34 (s, 9H); HPLC ret. time 2.19 min, 10-99% CH₃CN, 5 min run; ESI-MS 208.2 m/z (MH⁺).

### Example 5

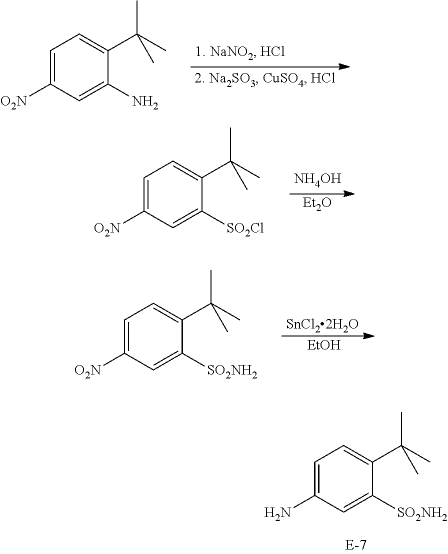

### 2-tert-Butyl-5-nitrobenzene-1-sulfonyl chloride

A suspension of 2-tert-butyl-5-nitrobenzenamine (0.971 g, 5 mmol) in conc. HCl (5 mL) was cooled to 5-10° C. and a solution of NaNO₂ (0.433 g, 6.3 mmol) in H₂O (0.83 mL) was added dropwise. Stirring was continued for 0.5 h, after which the mixture was vacuum filtered. The filtrate was added, simultaneously with a solution of Na₂SO₃ (1.57 g, 12.4 mmol) in H₂O (2.7 mL), to a stirred solution of CuSO₄ (0.190 g, 0.76 mmol) and Na₂SO₃ (1.57 g, 12.4 mmol) in HCl (11.7 mL) and H₂O (2.7 mL) at 3-5° C. Stirring was continued for 0.5 h and the resulting precipitate was filtered off, washed with water and dried to give 2-tert-butyl-5-nitrobenzene-1-sulfonyl chloride (0.235 g, 17%). ¹H NMR. (400 MHz, DMSO-d₆) δ 9.13 (d, J=2.5 Hz, 1H), 8.36 (dd, J=8.9, 2.5 Hz, 1H), 7.88 (d, J=8.9 Hz, 1H), 1.59 (s, 9H).

### 2-tert-Butyl-5-nitrobenzene-1-sulfonamide

To a solution of 2-tert-butyl-5-nitrobenzene-1-sulfonyl chloride (100 mg, 0.36 mmol) in ether (2 mL) was added aqueous NH₄OH (128 μL, 3.6 mmol) at 0° C. The mixture was stirred at room temperature overnight, diluted with water and extracted with ether. The combined ether extracts were washed with brine and dried over Na₂SO₄. After removal of solvent, the residue was purified by column chromatography (0-50% EtOAc-Hexane) to give 2-tert-butyl-5-nitrobenzene-1-sulfonamide (31.6 mg, 34%).

### E-7; 2-tert-Butyl-5-aminobenzene-1-sulfonamide

A solution of 2-tert-butyl-5-nitrobenzene-1-sulfonamide (32 mg, 0.12 mmol) and SnCl₂.2H₂O (138 mg, 0.61 mmol) in EtOH (1.5 mL) was heated in microwave oven at 100° C. for 30 min. The mixture was diluted with EtOAc and water, basified with sat. NaHCO₃ and filtered through Celite. The organic layer was separated from water and dried over Na₂SO₄. Solvent was removed by evaporation to provide 2-tert-butyl-5-aminobenzene-1-sulfonamide (E-7) (28 mg, 100%), which was used without further purification. HPLC ret. time 1.99 min, 10-99% CH₃CN, 5 min run; ESI-MS 229.3 m/z (MH⁺).

### Example 6

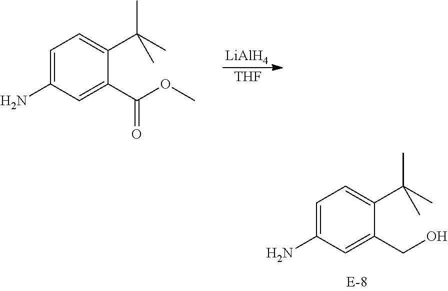

### E-8; (2-tert-Butyl-5-aminophenyl)methanol

To a solution of methyl 2-tert-butyl-5-aminobenzoate (159 mg, 0.72 mmol) in THF (5 mL) was added dropwise LiAlH₄ (1.4 mL, 1M in THF, 1.4 mmol) at 0° C. The reaction mixture was refluxed for 2 h, diluted with H₂O and extracted with EtOAc. The combined organic layers were washed with brine and dried over MgSO₄. After filtration, the filtrate was concentrated to give (2-tert-butyl-5-aminophenyl)methanol (E-8) (25 mg, 20%), which was used without further purification. ¹H NMR (400 MHz, CDCl₃) δ 7.17 (d, J=8.5 Hz, 1H), 6.87 (d, J=2.6 Hz, 1H), 6.56 (dd, J=8.4, 2.7 Hz, 1H), 4.83 (s, 2H), 1.36 (s, 9H).

### Example 7

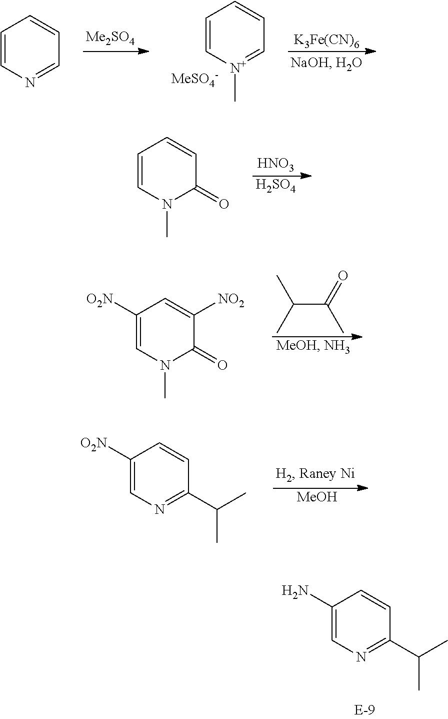

### 1-Methyl-pyridinium monomethyl sulfuric acid salt

Methyl sulfate (30 mL, 39.8 g, 0.315 mol) was added dropwise to dry pyridine (25.0 g, 0.316 mol) added dropwise. The mixture was stirred at room temperature for 10 min, then at 100° C. for 2 h. The mixture was cooled to room temperature to give crude 1-methyl-pyridinium monomethyl sulfuric acid salt (64.7 g, quant.), which was used without further purification.

### 1-Methyl-2-pyridone

A solution of 1-methyl-pyridinium monomethyl sulfuric acid salt (50 g, 0.243 mol) in water (54 mL) was cooled to 0° C. Separate solutions of potassium ferricyanide (160 g, 0.486 mol) in water (320 mL) and sodium hydroxide (40 g, 1.000 mol) in water (67 mL) were prepared and added dropwise from two separatory funnels to the well-stirred solution of 1-methyl-pyridinium monomethyl sulfuric acid salt, at such a rate that the temperature of reaction mixture did not rise above 10° C. The rate of addition of these two solutions was regulated so that all the sodium hydroxide solution had been introduced into the reaction mixture when one-half of the potassium Ferric Cyanide solution had been added. After addition was complete, the reaction mixture was allowed to warm to room temperature and stirred overnight. Dry sodium carbonate (91.6 g) was added, and the mixture was stirred for 10 min. The organic layer was separated, and the aqueous layer was extracted with CH₂Cl₂ (100 mL×3). The combined organic layers were dried and concentrated to yield 1-methyl-2-pyridone (25.0 g, 94%), which was used without further purification.

### 1-Methyl-3,5-dinitro-2-pyridone

1-Methyl-2-pyridone (25.0 g, 0.229 mol) was added to sulfuric acid (500 mL) at 0° C. After stirring for 5 min., nitric acid (200 mL) was added dropwise at 0° C. After addition, the reaction temperature was slowly raised to 100° C., and then maintained for 5 h. The reaction mixture was poured into ice, basified with potassium carbonate to pH 8 and extracted with CH₂Cl₂ (100 mL×3). The combined organic layers were dried over Na₂SO₄ and concentrated to yield 1-methyl-3,5-dinitro-2-pyridone (12.5 g, 28%), which was used without further purification.

### 2-Isopropyl-5-nitro-pyridine

To a solution of 1-methyl-3,5-dinitro-2-pyridone (8.0 g, 40 mmol) in methyl alcohol (20 mL) was added dropwise 3-methyl-2-butanone (5.1 mL, 48 mmol), followed by ammonia solution in methyl alcohol (10.0 g, 17%, 100 mmol). The reaction mixture was heated at 70° C. for 2.5 h under atmospheric pressure. The solvent was removed under vacuum and the residual oil was dissolved in CH₂Cl₂, and then filtered. The filtrate was dried over Na₂SO₄ and concentrated to afford 2-isopropyl-5-nitro-pyridine (1.88 g, 28%).

### E-9; 2-Isopropyl-5-amino-pyridine

2-Isopropyl-5-nitro-pyridine (1.30 g, 7.82 mmol) was dissolved in methyl alcohol (20 mL), and Raney Ni (0.25 g) was added. The mixture was stirred under H₂ (1 atm) at room temperature for 2 h. The catalyst was filtered off, and the filtrate was concentrated under vacuum to give 2-isopropyl-5-amino-pyridine (E-9) (0.55 g, 52%). ¹H NMR (CDCl₃) δ 8.05 (s, 1H), 6.93-6.99 (m, 2H), 3.47 (br s, 2H), 2.92-3.02 (m, 1H), 1.24-1.26 (m, 6H). ESI-MS 137.2 m/z (MH⁺).

### Example 8

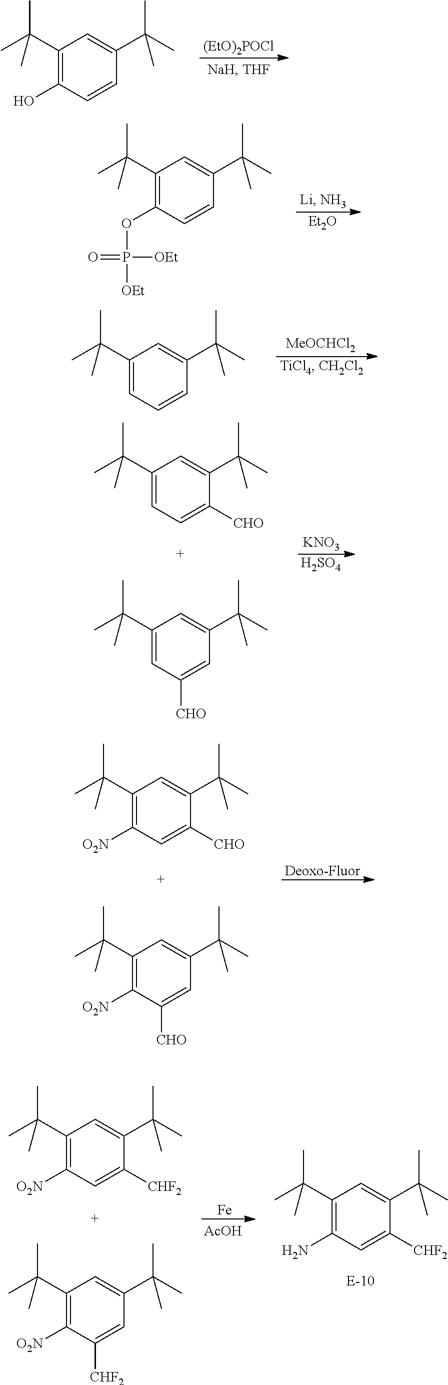

### Phosphoric acid 2,4-di-tert-butyl-phenyl ester diethyl ester

To a suspension of NaH (60% in mineral oil, 6.99 g, 174.7 mmol) in THF (350 mL) was added dropwise a solution of 2,4-di-tert-butylphenol (35 g, 169.6 mmol) in THF (150 mL) at 0° C. The mixture was stirred at 0° C. for 15 min and then phosphorochloridic acid diethyl ester (30.15 g, 174.7 mmol) was added dropwise at 0° C. After addition, the mixture was stirred at this temperature for 15 min. The reaction was quenched with sat. NH₄Cl (300 mL). The organic layer was separated and the aqueous phase was extracted with Et₂O (350 mL×2). The combined organic layers were washed with brine, dried over anhydrous Na₂SO₄ and concentrated under vacuum to give crude phosphoric acid 2,4-di-tert-butyl-phenyl ester diethyl ester as a yellow oil (51 g, contaminated with some mineral oil), which was used directly in the next step.

### 1,3-Di-tert-butyl-benzene

To NH₃ (liquid, 250 mL) was added a solution of phosphoric acid 2,4-di-tert-butyl-phenyl ester diethyl ester (51 g, crude from last step, about 0.2 mol) in Et₂O (anhydrous, 150 mL) at −78° C. under N₂ atmosphere. Lithium metal was added to the solution in small pieces until a blue color persisted. The reaction mixture was stirred at −78° C. for 15 min and then quenched with sat. NH₄Cl solution until the mixture turned colorless. Liquid NH₃ was evaporated and the residue was dissolved in water, extracted with Et₂O (300 mL×2). The combined organic phases were dried over Na₂SO₄ and concentrated to give crude 1,3-di-tert-butyl-benzene as a yellow oil (30.4 g, 94% over 2 steps, contaminated with some mineral oil), which was used directly in next step.

### 2,4-Di-tert-butyl-benzaldehyde and 3,5-di-tert-butyl-benzaldehyde

To a stirred solution of 1,3-di-tert-butyl-benzene (30 g, 157.6 mmol) in dry CH₂Cl₂ (700 mL) was added TiCl₄ (37.5 g, 197 mmol) at 0° C., and followed by dropwise addition of MeOCHCl₂ (27.3 g, 236.4 mmol). The reaction was allowed to warm to room temperature and stirred for 1 h. The mixture was poured into ice-water and extracted with CH₂Cl₂. The combined organic phases were washed with NaHCO₃ and brine, dried over Na₂SO₄ and concentrated. The residue was purified by column chromatography (petroleum ether) to give a mixture of 2,4-di-tert-butyl-benzaldehyde and 3,5-di-tert-butyl-benzaldehyde (21 g, 61%).

### 2,4-Di-tert-butyl-5-nitro-benzaldehyde and 3,5-di-tert-butyl-2-nitro-benzaldehyde

To a mixture of 2,4-di-tert-butyl-benzaldehyde and 3,5-di-tert-butyl-benzaldehyde in H₂SO₄ (250 mL) was added KNO₃ (7.64 g, 75.6 mmol) in portions at 0° C. The reaction mixture was stirred at this temperature for 20 min and then poured into crushed ice. The mixture was basified with NaOH solution to pH 8 and extracted with Et₂O (10 mL×3). The combined organic layers were washed with water and brine and concentrated. The residue was purified by column chromatography (petroleum ether) to give a mixture of 2,4-di-tert-butyl-5-nitro-benzaldehyde and 3,5-di-tert-butyl-2-nitro-benzaldehyde (2:1 by NMR) as a yellow solid (14.7 g, 82%). After further purification by column chromatography (petroleum ether), 2,4-di-tert-butyl-5-nitro-benzaldehyde (2.5 g, contains 10% 3,5-di-tert-butyl-2-nitro-benzaldehyde) was isolated.

### 1,5-Di-tert-butyl-2-difluoromethyl-4-nitro-benzene and 1,5-Di-tert-butyl-3-difluoromethyl-2-nitro-benzene

2,4-Di-tert-butyl-5-nitro-benzaldehyde (2.4 g, 9.11 mmol, contaminated with 10% 3,5-di-tert-butyl-2-nitro-benzaldehyde) in neat deoxofluor solution was stirred at room temperature for 5 h. The reaction mixture was poured into cooled sat. NaHCO₃ solution and extracted with dichloromethane. The combined organics were dried over Na₂SO₄, concentrated and purified by column chromatography (petroleum ether) to give 1,5-di-tert-butyl-2-difluoromethyl-4-nitro-benzene (1.5 g) and a mixture of 1,5-di-tert-butyl-2-difluoromethyl-4-nitro-benzene and 1,5-di-tert-butyl-3-difluoromethyl-2-nitro-benzene (0.75 g, contains 28% 1,5-di-tert-butyl-3-difluoromethyl-2-nitro-benzene).

### E-10; 1,5-Di-tert-butyl-2-difluoromethyl-4-amino-benzene

To a suspension of iron powder (5.1 g, 91.1 mmol) in 50% acetic acid (25 ml) was added 1,5-di-tert-butyl-2-difluoromethyl-4-nitro-benzene (1.3 g, 4.56 mmol). The reaction mixture was heated at 115° C. for 15 min. Solid was filtered off was washed with acetic acid and CH₂Cl₂. The combined filtrate was concentrated and treated with HCl/MeOH. The precipitate was collected via filtration, washed with MeOH and dried to give 1,5-Di-tert-butyl-2-difluoromethyl-4-amino-benzene HCl salt (E-10) as a white solid (1.20 g, 90%). ¹H NMR (DMSO-d₆) δ 7.35-7.70 (t, J=53.7 Hz, 1H), 7.56 (s, 1H), 7.41 (s, 1H), 1.33-1.36 (d, J=8.1 Hz, 1H); ESI-MS 256.3 m/z (MH⁺).

### Example 9

### General Scheme

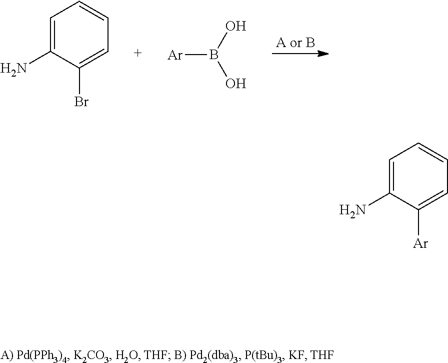

Method A

In a 2-dram vial, 2-bromoaniline (100 mg, 0.58 mmol) and the corresponding aryl boronic acid (0.82 mmol) were dissolved in THF (1 mL). H₂O (500 μL) was added followed by K₂CO₃ (200 mg, 1.0 mmol) and Pd(PPh₃)₄ (100 mg, 0.1 mmol). The vial was purged with argon and sealed. The vial was then heated at 75° C. for 18 h. The crude sample was diluted in EtOAc and filtered through a silica gel plug. The organics were concentrated via Savant Speed-vac. The crude amine was used without further purification.

Method B

In a 2-dram vial, the corresponding aryl boronic acid (0.58 mmol) was added followed by KF (110 mg, 1.9 mmol). The solids were suspended in THF (2 mL), and then 2-bromoaniline (70 μL, 0.58 mmol) was added. The vial was purged with argon for 1 min. P(^{t}Bu)₃ (100 μL, 10% sol. in hexanes) was added followed by Pd₂(dba)₃ (900 μL, 0.005 M in THF). The vial was purged again with argon and sealed. The vial was agitated on an orbital shaker at room temperature for 30 min and heated in a heating block at 80° C. for 16 h. The vial was then cooled to 20° C. and the suspension was passed through a pad of Celite. The pad was washed with EtOAc (5 mL). The organics were combined and concentrated under vacuum to give a crude amine that was used without further purification.

The table below includes the amines made following the general scheme above.

| Product | Name | Method |
|---|---|---|
| F-1 | 4′-Methyl-biphenyl-2-ylamine | A |
| F-2 | 3′-Methyl-biphenyl-2-ylamine | A |
| F-3 | 2′-Methyl-biphenyl-2-ylamine | A |
| F-4 | 2′,3′-Dimethyl-biphenyl-2-ylamine | A |
| F-5 | (2′-Amino-biphenyl-4-yl)-methanol | A |
| F-6 | N*4′*,N*4′*-Dimethyl-biphenyl-2,4′-diamine | B |
| F-7 | 2′-Trifluoromethyl-biphenyl-2-ylamine | B |
| F-8 | (2′-Amino-biphenyl-4-yl)-acetonitrile | A |
| F-9 | 4′-Isobutyl-biphenyl-2-ylamine | A |
| F-10 | 3′-Trifluoromethyl-biphenyl-2-ylamine | B |
| F-11 | 2-Pyridin-4-yl-phenylamine | B |
| F-12 | 2-(1H-Indol-5-yl)-phenylamine | B |
| F-13 | 3′,4′-Dimethyl-biphenyl-2-ylamine | A |
| F-14 | 4′-Isopropyl-biphenyl-2-ylamine | A |
| F-15 | 3′-Isopropyl-biphenyl-2-ylamine | A |
| F-16 | 4′-Trifluoromethyl-biphenyl-2-ylamine | B |
| F-17 | 4′-Methoxy-biphenyl-2-ylamine | B |
| F-18 | 3′-Methoxy-biphenyl-2-ylamine | B |
| F-19 | 2-Benzo[1,3]dioxol-5-yl-phenylamine | B |
| F-20 | 3′-Ethoxy-biphenyl-2-ylamine | B |
| F-21 | 4′-Ethoxy-biphenyl-2-ylamine | B |
| F-22 | 2′-Ethoxy-biphenyl-2-ylamine | B |
| F-23 | 4′-Methylsulfanyl-biphenyl-2-ylamine | B |
| F-24 | 3′,4′-Dimethoxy-biphenyl-2-ylamine | B |
| F-25 | 2′,6′-Dimethoxy-biphenyl-2-ylamine | B |
| F-26 | 2′,5′-Dimethoxy-biphenyl-2-ylamine | B |
| F-27 | 2′,4′-Dimethoxy-biphenyl-2-ylamine | B |
| F-28 | 5′-Chloro-2′-methoxy-biphenyl-2-ylamine | B |
| F-29 | 4′-Trifluoromethoxy-biphenyl-2-ylamine | B |
| F-30 | 3′-Trifluoromethoxy-biphenyl-2-ylamine | B |
| F-31 | 4′-Phenoxy-biphenyl-2-ylamine | B |
| F-32 | 2′-Fluoro-3′-methoxy-biphenyl-2-ylamine | B |
| F-33 | 2′-Phenoxy-biphenyl-2-ylamine | B |
| F-34 | 2-(2,4-Dimethoxy-pyrimidin-5-yl)-phenylamine | B |
| F-35 | 5′-Isopropyl-2′-methoxy-biphenyl-2-ylamine | B |
| F-36 | 2′-Trifluoromethoxy-biphenyl-2-ylamine | B |
| F-37 | 4′-Fluoro-biphenyl-2-ylamine | B |
| F-38 | 3′-Fluoro-biphenyl-2-ylamine | B |
| F-39 | 2′-Fluoro-biphenyl-2-ylamine | B |
| F-40 | 2′-Amino-biphenyl-3-carbonitrile | B |
| F-41 | 4′-Fluoro-3′-methyl-biphenyl-2-ylamine | B |
| F-42 | 4′-Chloro-biphenyl-2-ylamine | B |
| F-43 | 3′-Chloro-biphenyl-2-ylamine | B |
| F-44 | 3′,5′-Difluoro-biphenyl-2-ylamine | B |
| F-45 | 2′,3′-Difluoro-biphenyl-2-ylamine | B |
| F-46 | 3′,4′-Difluoro-biphenyl-2-ylamine | B |
| F-47 | 2′,4′-Difluoro-biphenyl-2-ylamine | B |
| F-48 | 2′,5′-Difluoro-biphenyl-2-ylamine | B |
| F-49 | 3′-Chloro-4′-fluoro-biphenyl-2-ylamine | B |
| F-50 | 3′,5′-Dichloro-biphenyl-2-ylamine | B |
| F-51 | 2′,5′-Dichloro-biphenyl-2-ylamine | B |
| F-52 | 2′,3′-Dichloro-biphenyl-2-ylamine | B |
| F-53 | 3′,4′-Dichloro-biphenyl-2-ylamine | B |
| F-54 | 2′-Amino-biphenyl-4-carboxylic acid methyl ester | B |
| F-55 | 2′-Amino-biphenyl-3-carboxylic acid methyl ester | B |
| F-56 | 2′-Methylsulfanyl-biphenyl-2-ylamine | B |
| F-57 | N-(2′-Amino-biphenyl-3-yl)-acetamide | B |
| F-58 | 4′-Methanesulfinyl-biphenyl-2-ylamine | B |
| F-59 | 2′,4′-Dichloro-biphenyl-2-ylamine | B |
| F-60 | 4′-Methanesulfonyl-biphenyl-2-ylamine | B |
| F-61 | 2′-Amino-biphenyl-2-carboxylic acid isopropyl ester | B |
| F-62 | 2-Furan-2-yl-phenylamine | B |
| F-63 | 1-[5-(2-Amino-phenyl)-thiophen-2-yl]-ethanone | B |
| F-64 | 2-Benzo[b]thiophen-2-yl-phenylamine | B |
| F-65 | 2-Benzo[b]thiophen-3-yl-phenylamine | B |
| F-66 | 2-Furan-3-yl-phenylamine | B |
| F-67 | 2-(4-Methyl-thiophen-2-yl)-phenylamine | B |
| F-68 | 5-(2-Amino-phenyl)-thiophene-2-carbonitrile | B |

### Example 10

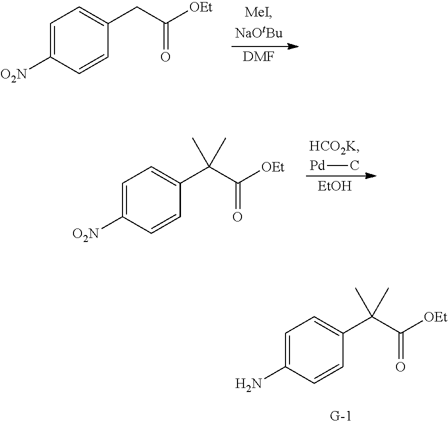

### Ethyl 2-(4-nitrophenyl)-2-methylpropanoate

Sodium t-butoxide (466 mg, 4.85 mmol) was added to DMF (20 mL) at 0° C. The cloudy solution was re-cooled to 5° C. Ethyl 4-nitrophenylacetate (1.0 g, 4.78 mmol) was added. The purple slurry was cooled to 5° C. and methyl iodide (0.688 mL, 4.85 mmol) was added over 40 min. The mixture was stirred at 5-10° C. for 20 min, and then re-charged with sodium t-butoxide (466 mg, 4.85 mmol) and methyl iodide (0.699 mL, 4.85 mmol). The mixture was stirred at 5-10° C. for 20 min and a third charge of sodium t-butoxide (47 mg, 0.48 mmol) was added followed by methyl iodide (0.057 mL, 0.9 mmol). Ethyl acetate (100 mL) and HCl (0.1N, 50 mL) were added. The organic layer was separated, washed with brine and dried over Na₂SO₄. After filtration, the filtrate was concentrated to provide ethyl 2-(4-nitrophenyl)-2-methylpropanoate (900 mg, 80%), which was used without further purification.

### G-1; Ethyl 2-(4-aminophenyl)-2-methylpropanoate

A solution of ethyl 2-(4-nitrophenyl)-2-methylpropanoate (900 mg, 3.8 mmol) in EtOH (10 mL) was treated with 10% Pd—C (80 mg) and heated to 45° C. A solution of potassium formate (4.10 g, 48.8 mmol) in H₂O (11 mL) was added over a period of 15 min. The reaction mixture was stirred at 65° C. for 2 h and then treated with additional 300 mg of Pd/C. The reaction was stirred for 1.5 h and then filtered through Celite. The solvent volume was reduced by approximately 50% under reduced pressure and extracted with EtOAc. The organic layers were dried over Na₂SO₄ and the solvent was removed under reduced pressure to yield ethyl 2-(4-aminophenyl)-2-methylpropanoate (G-1) (670 mg, 85%). ¹H NMR (400 MHz, CDCl₃) δ 7.14 (d, J=8.5 Hz, 2H), 6.65 (d, J=8.6 Hz, 2H), 4.10 (q, J=7.1 Hz, 2H), 1.53 (s, 6H), 1.18 (t, J=7.1 Hz, 3H).

### Example 11

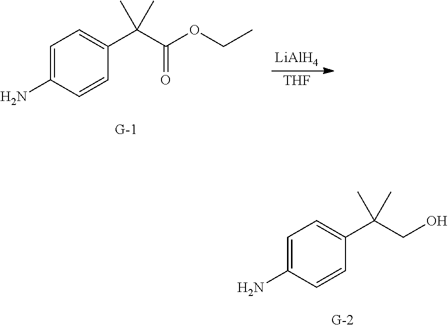

### G-2; 2-(4-Aminophenyl)-2-methylpropan-1-ol

A solution of ethyl 2-(4-aminophenyl)-2-methylpropanoate (30 mg, 0.145 mmol) in THF (1 mL) was treated with LiAlH₄ (1M solution in THF, 0.226 mL, 0.226 mmol) at 0° C. and stirred for 15 min. The reaction was treated with 0.1N NaOH, extracted with EtOAc and the organic layers were dried over Na₂SO₄. The solvent was removed under reduced pressure to yield 2-(4-aminophenyl)-2-methylpropan-1-ol (G-2), which was used without further purification: ¹H NMR (400 MHz, CDCl₃) δ 7.17 (d, J=8.5 Hz, 2H), 6.67 (d, J=8.5 Hz, 2H), 3.53 (s, 2H), 1.28 (s, 6H).

### Example 12

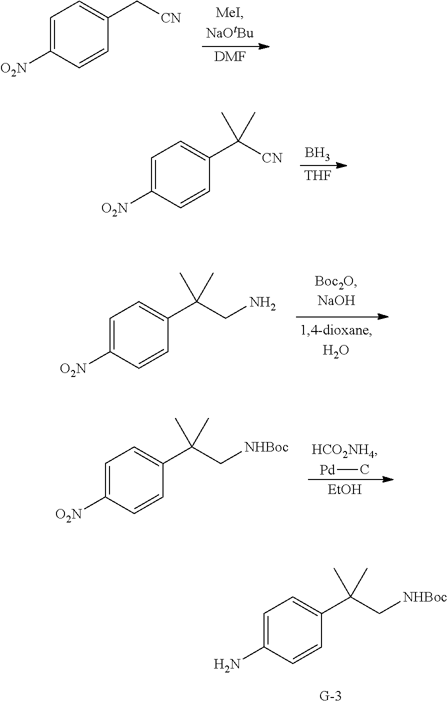

### 2-methyl-2-(4-nitrophenyl)propanenitrile

A suspension of sodium tert-butoxide (662 mg, 6.47 mmol) in DMF (20 mL) at 0° C. was treated with 4-nitrophenylacetonitrile (1000 mg, 6.18 mmol) and stirred for 10 min. Methyl iodide (400 μL, 6.47 mmol) was added dropwise over 15 min. The solution was stirred at 0-10° C. for 15 min and then at room temperature for additional 15 min. To this purple solution was added sodium tert-butoxide (662 mg, 6.47 mmol) and the solution was stirred for 15 min. Methyl iodide (400 μL, 6.47 mmol) was added dropwise over 15 min and the solution was stirred overnight. Sodium tert-butoxide (192 mg, 1.94 mmol) was added and the reaction was stirred at 0° C. for 10 minutes. Methyl iodide (186 μL, 2.98 mmol) was added and the reaction was stirred for 1 h. The reaction mixture was then partitioned between 1N HCl (50 mL) and EtOAc (75 mL). The organic layer was washed with 1N HCl and brine, dried over Na₂SO₄ and concentrated to yield 2-methyl-2-(4-nitrophenyl)propanenitrile as a green waxy solid (1.25 g, 99%). ¹H NMR (400 MHz, CDCl₃) δ 8.24 (d, J=8.9 Hz, 2H), 7.66 (d, J=8.9 Hz, 2H), 1.77 (s, 6H).

### 2-Methyl-2-(4-nitrophenyl)propan-1-amine

To a cooled solution of 2-methyl-2-(4-nitrophenyl)propanenitrile (670 mg, 3.5 mmol) in THF (15 mL) was added BH₃ (1M in THF, 14 mL, 14 mmol) dropwise at 0° C. The mixture was warmed to room temperature and heated at 70° C. for 2 h. 1N HCl solution (2 mL) was added, followed by the addition of NaOH until pH >7. The mixture was extracted with ether and ether extract was concentrated to give 2-methyl-2-(4-nitrophenyl)propan-1-amine (610 mg, 90%), which was used without further purification. ¹H NMR (400 MHz, CDCl₃) δ 8.20 (d, J=9.0 Hz, 2H), 7.54 (d, J=9.0 Hz, 2H), 2.89 (s, 2H), 1.38 (s, 6H).

### tert-Butyl 2-methyl-2-(4-nitrophenyl)propylcarbamate

To a cooled solution of 2-methyl-2-(4-nitrophenyl)propan-1-amine (600 mg, 3.1 mmol) and 1N NaOH (3 mL, 3 mmol) in 1,4-dioxane (6 mL) and water (3 mL) was added Boc₂O (742 mg, 3.4 mmol) at 0° C. The reaction was allowed to warm to room temperature and stirred overnight. The reaction was made acidic with 5% KHSO₄ solution and then extracted with ethyl acetate. The organic layer was dried over MgSO₄ and concentrated to give tert-butyl 2-methyl-2-(4-nitrophenyl)propylcarbamate (725 mg, 80%), which was used without further purification. ¹H NMR (400 MHz, CDCl₃) δ 8.11 (d, J=8.9 Hz, 2H), 7.46 (d, J=8.8 Hz, 2H), 3.63 (s, 2H), 1.31-1.29 (m, 15H).

### G-3; tert-Butyl 2-methyl-2-(4-aminophenyl)propylcarbamate

To a refluxing solution of tert-butyl 2-methyl-2-(4-nitrophenyl)propylcarbamate (725 mg, 2.5 mmol) and ammonium formate (700 mg, 10.9 mmol) in EtOH (25 mL) was added Pd-5% wt on carbon (400 mg). The mixture was refluxed for 1 h, cooled and filtered through Celite. The filtrate was concentrated to give tert-butyl 2-methyl-2-(4-aminophenyl)propylcarbamate (G-3) (550 mg, 83%), which was used without further purification. ¹H NMR (400 MHz, DMSO-d₆) δ 6.99 (d, J=8.5 Hz, 2H), 6.49 (d, J=8.6 Hz, 2H), 4.85 (s, 2H), 3.01 (d, J=6.3 Hz, 2H), 1.36 (s, 9H), 1.12 (s, 6H); HPLC ret. time 2.02 min, 10-99% CH₃CN, 5 min run; ESI-MS 265.2 m/z (MH⁺).

### Example 13

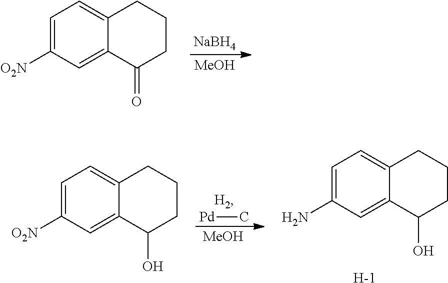

### 7-Nitro-1,2,3,4-tetrahydro-naphthalen-1-ol

7-Nitro-3,4-dihydro-2H-naphthalen-1-one (200 mg, 1.05 mmol) was dissolved in methanol (5 mL) and NaBH₄ ((78 mg, 2.05 mmol) was added in portions. The reaction was stirred at room temperature for 20 min and then concentrated and purified by column chromatography (10-50% ethyl acetate-hexanes) to yield 7-nitro-1,2,3,4-tetrahydro-naphthalen-1-ol (163 mg, 80%). ¹H NMR (400 MHz, CD₃CN) δ 8.30 (d, J=2.3 Hz, 1H), 8.02 (dd, J=8.5, 2.5 Hz, 1H), 7.33 (d, J=8.5 Hz, 1H), 4.76 (t, J=5.5 Hz, 1H), 2.96-2.80 (m, 2H), 2.10-1.99 (m, 2H), 1.86-1.77 (m, 2H); HPLC ret. time 2.32 min, 10-99% CH₃CN, 5 min run.

### H-1; 7-Amino-1,2,3,4-tetrahydro-naphthalen-1-ol

7-nitro-1,2,3,4-tetrahydro-naphthalen-1-ol (142 mg, 0.73 mmol) was dissolved in methanol (10 mL) and the flask was flushed with N₂ (g). 10% Pd—C (10 mg) was added and the reaction was stirred under H₂ (1 atm) at room temperature overnight. The reaction was filtered and the filtrate concentrated to yield 7-amino-1,2,3,4-tetrahydro-naphthalen-1-ol (H-1) (113 mg, 95%). HPLC ret. time 0.58 min, 10-99% CH₃CN, 5 min run; ESI-MS 164.5 m/z (MH⁺).

### Example 14

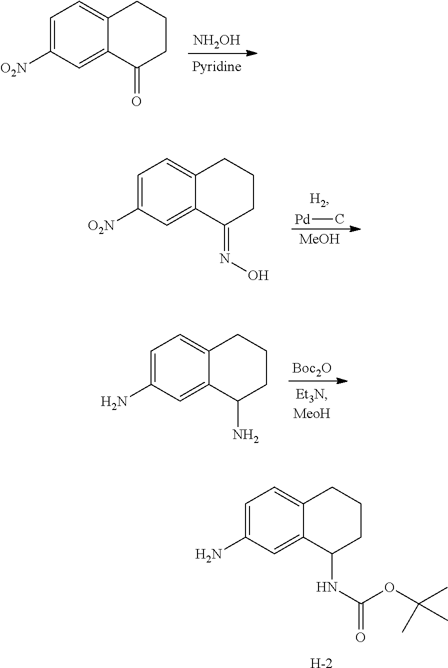

### 7-Nitro-3,4-dihydro-2H-naphthalen-1-one oxime

To a solution of 7-nitro-3,4-dihydro-2H-naphthalen-1-one (500 mg, 2.62 mmol) in pyridine (2 mL) was added hydroxylamine solution (1 mL, ˜50% solution in water). The reaction was stirred at room temperature for 1 h, then concentrated and purified by column chromatography (10-50% ethyl acetate-hexanes) to yield 7-nitro-3,4-dihydro-2H-naphthalen-1-one oxime (471 mg, 88%). HPLC ret. time 2.67 min, 10-99% CH₃CN, 5 min run; ESI-MS 207.1 m/z (MH⁺).

### 1,2,3,4-Tetrahydro-naphthalene-1,7-diamine

7-Nitro-3,4-dihydro-2H-naphthalen-1-one oxime (274 mg, 1.33 mmol) was dissolved in methanol (10 mL) and the flask was flushed with N₂ (g). 10% Pd—C (50 mg) was added and the reaction was stirred under H₂ (1 atm) at room temperature overnight. The reaction was filtered and the filtrate was concentrated to yield 1,2,3,4-tetrahydro-naphthalene-1,7-diamine (207 mg, 96%). ¹H NMR (400 MHz, DMSO-d₆) δ 6.61-6.57 (m, 2H), 6.28 (dd, J=8.0, 2.4 Hz, 1H), 4.62 (s, 2H), 3.58 (m, 1H), 2.48-2.44 (m, 2H), 1.78-1.70 (m, 2H), 1.53-1.37 (m, 2H).

### H-2; (7-Amino-1,2,3,4-tetrahydro-naphthalen-1-yl)-carbamic acid tert-butyl ester

To a solution of 1,2,3,4-tetrahydro-naphthalene-1,7-diamine (154 mg, 0.95 mmol) and triethylamine (139 μL, 1.0 mmol) in methanol (2 mL) cooled to 0° C. was added di-tert-butyl dicarbonate (207 mg, 0.95 mmol). The reaction was stirred at 0° C. and then concentrated and purified by column chromatography (5-50% methanol-dichloromethane) to yield (7-amino-1,2,3,4-tetrahydro-naphthalen-1-yl)-carbamic acid tert-butyl ester (H-2) (327 mg, quant.). HPLC ret. time 1.95 min, 10-99% CH₃CN, 5 min run; ESI-MS 263.1 m/z (MH⁺).

### Example 15

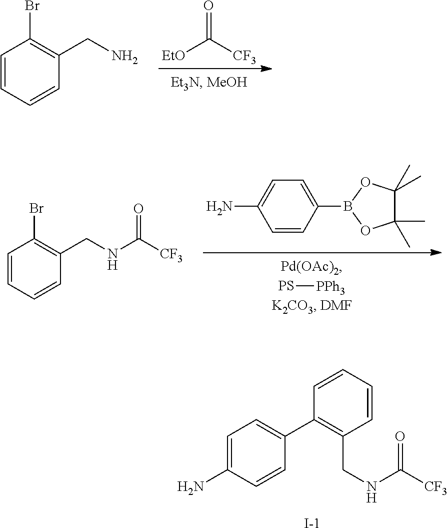

### N-(2-Bromo-benzyl)-2,2,2-trifluoro-acetamide

To a solution of 2-bromobenzylamine (1.3 mL, 10.8 mmol) in methanol (5 mL) was added ethyl trifluoroacetate (1.54 mL, 21.6 mmol) and triethylamine (1.4 mL, 10.8 mmol) under a nitrogen atmosphere. The reaction was stirred at room temperature for 1 h. The reaction mixture was then concentrated under vacuum to yield N-(2-bromo-benzyl)-2,2,2-trifluoro-acetamide (3.15 g, quant.). HPLC ret. time 2.86 min, 10-99% CH₃CN, 5 min run; ESI-MS 283.9 m/z (MH⁺).

### I-1; N-(4′-Amino-biphenyl-2-ylmethyl)-2,2,2-trifluoro-acetamide

A mixture of N-(2-bromo-benzyl)-2,2,2-trifluoro-acetamide (282 mg, 1.0 mmol), 4-(4,4,5,5-tetramethyl-1,3,2-dioxaborolan-2-yl)aniline (284 mg, 1.3 mmol), Pd(OAc)₂ (20 mg, 0.09 mmol) and PS—PPh₃ (40 mg, 3 mmol/g, 0.12 mmol) was dissolved in DMF (5 mL) and 4M K₂CO₃ solution (0.5 mL) was added. The reaction was heated at 80° C. overnight. The mixture was filtered, concentrated and purified by column chromatography (0-50% ethyl acetate-hexanes) to yield N-(4′-amino-biphenyl-2-ylmethyl)-2,2,2-trifluoro-acetamide (I-1) (143 mg, 49%). HPLC ret. time 1.90 min, 10-99% CH₃CN, 5 min run; ESI-MS 295.5 m/z (MH⁺).

Commercially available amines

| Amine | Name |
|---|---|
| J-1 | 2-methoxy-5-methylbenzenamine |
| J-2 | 2,6-diisopropylbenzenamine |
| J-3 | pyridin-2-amine |
| J-4 | 4-pentylbenzenamine |
| J-5 | isoquinolin-3-amine |
| J-6 | aniline |
| J-7 | 4-phenoxybenzenamine |
| J-8 | 2-(2,3-dimethylphenoxy)pyridin-3-amine |
| J-9 | 4-ethynylbenzenamine |
| J-10 | 2-sec-butylbenzenamine |
| J-11 | 2-amino-4,5-dimethoxybenzonitrile |
| J-12 | 2-tert-butylbenzenamine |
| J-13 | 1-(7-amino-3,4-dihydroisoquinolin-2(1H)-yl)ethanone |
| J-14 | 4-(4-methyl-4H-1,2,4-triazol-3-yl)benzenamine |
| J-15 | 2′-Aminomethyl-biphenyl-4-ylamine |
| J-16 | 1H-Indazol-6-ylamine |
| J-17 | 2-(2-methoxyphenoxy)-5-(trifluoromethyl)benzenamine |
| J-18 | 2-tert-butylbenzenamine |
| J-19 | 2,4,6-trimethylbenzenamine |
| J-20 | 5,6-dimethyl-1H-benzo[d]imidazol-2-amine |
| J-21 | 2,3-dihydro-1H-inden-4-amine |
| J-22 | 2-sec-butyl-6-ethylbenzenamine |
| J-23 | quinolin-5-amine |
| J-24 | 4-(benzyloxy)benzenamine |
| J-25 | 2′-Methoxy-biphenyl-2-ylamine |
| J-26 | benzo[c][1,2,5]thiadiazol-4-amine |
| J-27 | 3-benzylbenzenamine |
| J-28 | 4-isopropylbenzenamine |
| J-29 | 2-(phenylsulfonyl)benzenamine |
| J-30 | 2-methoxybenzenamine |
| J-31 | 4-amino-3-ethylbenzonitrile |
| J-32 | 4-methylpyridin-2-amine |
| J-33 | 4-chlorobenzenamine |
| J-34 | 2-(benzyloxy)benzenamine |
| J-35 | 2-amino-6-chlorobenzonitrile |
| J-36 | 3-methylpyridin-2-amine |
| J-37 | 4-aminobenzonitrile |
| J-38 | 3-chloro-2,6-diethylbenzenamine |
| J-39 | 3-phenoxybenzenamine |
| J-40 | 2-benzylbenzenamine |
| J-41 | 2-(2-fluorophenoxy)pyridin-3-amine |
| J-42 | 5-chloropyridin-2-amine |
| J-43 | 2-(trifluoromethyl)benzenamine |
| J-44 | (4-(2-aminophenyl)piperazin-1-yl)(phenyl)methanone |
| J-45 | 1H-benzo[d][1,2,3]triazol-5-amine |
| J-46 | 2-(1H-indol-2-yl)benzenamine |
| J-47 | 4-Methyl-biphenyl-3-ylamine |
| J-48 | pyridin-3-amine |
| J-49 | 3,4-dimethoxybenzenamine |
| J-50 | 3H-benzo[d]imidazol-5-amine |
| J-51 | 3-aminobenzonitrile |
| J-52 | 6-chloropyridin-3-amine |
| J-53 | o-toluidine |
| J-54 | 1H-indol-5-amine |
| J-55 | [1,2,4]triazolo[1,5-a]pyridin-8-amine |
| J-56 | 2-methoxypyridin-3-amine |
| J-57 | 2-butoxybenzenamine |
| J-58 | 2,6-dimethylbenzenamine |
| J-59 | 2-(methylthio)benzenamine |
| J-60 | 2-(5-methylfuran-2-yl)benzenamine |
| J-61 | 3-(4-aminophenyl)-3-ethylpiperidine-2,6-dione |
| J-62 | 2,4-dimethylbenzenamine |
| J-63 | 5-fluoropyridin-2-amine |
| J-64 | 4-cyclohexylbenzenamine |
| J-65 | 4-Amino-benzenesulfonamide |
| J-66 | 2-ethylbenzenamine |
| J-67 | 4-fluoro-3-methylbenzenamine |
| J-68 | 2,6-dimethoxypyridin-3-amine |
| J-69 | 4-tert-butylbenzenamine |
| J-70 | 4-sec-butylbenzenamine |
| J-71 | 5,6,7,8-tetrahydronaphthalen-2-amine |
| J-72 | 3-(Pyrrolidine-1-sulfonyl)-phenylamine |
| J-73 | 4-Adamantan-1-yl-phenylamine |
| J-74 | 3-amino-5,6,7,8-tetrahydronaphthalen-2-ol |
| J-75 | benzo[d][1,3]dioxol-5-amine |
| J-76 | 5-chloro-2-phenoxybenzenamine |
| J-77 | N1-tosylbenzene-1,2-diamine |
| J-78 | 3,4-dimethylbenzenamine |
| J-79 | 2-(trifluoromethylthio)benzenamine |
| J-80 | 1H-indol-7-amine |
| J-81 | 3-methoxybenzenamine |
| J-82 | quinolin-8-amine |
| J-83 | 2-(2,4-difluorophenoxy)pyridin-3-amine |
| J-84 | 2-(4-aminophenyl)acetonitrile |
| J-85 | 2,6-dichlorobenzenamine |
| J-86 | 2,3-dihydrobenzofuran-5-amine |
| J-87 | p-toluidine |
| J-88 | 2-methylquinolin-8-amine |
| J-89 | 2-tert-butylbenzenamine |
| J-90 | 3-chlorobenzenamine |
| J-91 | 4-tert-butyl-2-chlorobenzenamine |
| J-92 | 2-Amino-benzenesulfonamide |
| J-93 | 1-(2-aminophenyl)ethanone |
| J-94 | m-toluidine |
| J-95 | 2-(3-chloro-5-(trifluoromethyl)pyridin-2-yloxy)benzenamine |
| J-96 | 2-amino-6-methylbenzonitrile |
| J-97 | 2-(prop-1-en-2-yl)benzenamine |
| J-98 | 4-Amino-N-pyridin-2-yl-benzenesulfonamide |
| J-99 | 2-ethoxybenzenamine |
| J-100 | naphthalen-1-amine |
| J-101 | Biphenyl-2-ylamine |
| J-102 | 2-(trifluoromethyl)-4-isopropylbenzenamine |
| J-103 | 2,6-diethylbenzenamine |
| J-104 | 5-(trifluoromethyl)pyridin-2-amine |
| J-105 | 2-aminobenzamide |
| J-106 | 3-(trifluoromethoxy)benzenamine |
| J-107 | 3,5-bis(trifluoromethyl)benzenamine |
| J-108 | 4-vinylbenzenamine |
| J-109 | 4-(trifluoromethyl)benzenamine |
| J-110 | 2-morpholinobenzenamine |
| J-111 | 5-amino-1H-benzo[d]imidazol-2(3H)-one |
| J-112 | quinolin-2-amine |
| J-113 | 3-methyl-1H-indol-4-amine |
| J-114 | pyrazin-2-amine |
| J-115 | 1-(3-aminophenyl)ethanone |
| J-116 | 2-ethyl-6-isopropylbenzenamine |
| J-117 | 2-(3-(4-chlorophenyl)-1,2,4-oxadiazol-5-yl)benzenamine |
| J-118 | N-(4-amino-2,5-diethoxyphenyl)benzamide |
| J-119 | 5,6,7,8-tetrahydronaphthalen-1-amine |
| J-120 | 2-(1H-benzo[d]imidazol-2-yl)benzenamine |
| J-121 | 1,1-Dioxo-1H-1lambda*6*-benzo[b]thiophen-6-ylamine |
| J-122 | 2,5-diethoxybenzenamine |
| J-123 | 2-isopropyl-6-methylbenzenamine |
| J-124 | tert-butyl 5-amino-3,4-dihydroisoquinoline-2(1H)-carboxylate |
| J-125 | 2-(2-aminophenyl)ethanol |
| J-126 | (4-aminophenyl)methanol |
| J-127 | 5-methylpyridin-2-amine |
| J-128 | 2-(pyrrolidin-1-yl)benzenamine |
| J-129 | 4-propylbenzenamine |
| J-130 | 3,4-dichlorobenzenamine |
| J-131 | 2-phenoxybenzenamine |
| J-132 | Biphenyl-2-ylamine |
| J-133 | 2-chlorobenzenamine |
| J-134 | 2-amino-4-methylbenzonitrile |
| J-135 | (2-aminophenyl)(phenyl)methanone |
| J-136 | aniline |
| J-137 | 3-(trifluoromethylthio)benzenamine |
| J-138 | 2-(2,5-dimethyl-1H-pyrrol-1-yl)benzenamine |
| J-139 | 4-(Morpholine-4-sulfonyl)-phenylamine |
| J-140 | 2-methylbenzo[d]thiazol-5-amine |
| J-141 | 2-amino-3,5-dichlorobenzonitrile |
| J-142 | 2-fluoro-4-methylbenzenamine |
| J-143 | 6-ethylpyridin-2-amine |
| J-144 | 2-(1H-pyrrol-1-yl)benzenamine |
| J-145 | 2-methyl-1H-indol-5-amine |
| J-146 | quinolin-6-amine |
| J-147 | 1H-benzo[d]imidazol-2-amine |
| J-148 | 2-o-tolylbenzo[d]oxazol-5-amine |
| J-149 | 5-phenylpyridin-2-amine |
| J-150 | Biphenyl-2-ylamine |
| J-151 | 4-(difluoromethoxy)benzenamine |
| J-152 | 5-tert-butyl-2-methoxybenzenamine |
| J-153 | 2-(2-tert-butylphenoxy)benzenamine |
| J-154 | 3-aminobenzamide |
| J-155 | 4-morpholinobenzenamine |
| J-156 | 6-aminobenzo[d]oxazol-2(3H)-one |
| J-157 | 2-phenyl-3H-benzo[d]imidazol-5-amine |
| J-158 | 2,5-dichloropyridin-3-amine |
| J-159 | 2,5-dimethylbenzenamine |
| J-160 | 4-(phenylthio)benzenamine |
| J-161 | 9H-fluoren-1-amine |
| J-162 | 2-(4-aminophenyl)-1,1,1,3,3,3-hexafluoropropan-2-ol |
| J-163 | 4-bromo-2-ethylbenzenamine |
| J-164 | 4-methoxybenzenamine |
| J-165 | 3-(Piperidine-1-sulfonyl)-phenylamine |
| J-166 | quinoxalin-6-amine |
| J-167 | 6-(trifluoromethyl)pyridin-3-amine |
| J-168 | 3-(trifluoromethyl)-2-methylbenzenamine |
| J-169 | (2-aminophenyl)(phenyl)methanol |
| J-170 | aniline |
| J-171 | 6-methoxypyridin-3-amine |
| J-172 | 4-butylbenzenamine |
| J-173 | 3-(Morpholine-4-sulfonyl)-phenylamine |
| J-174 | 2,3-dimethylbenzenamine |
| J-175 | aniline |
| J-176 | Biphenyl-2-ylamine |
| J-177 | 2-(2,4-dichlorophenoxy)benzenamine |
| J-178 | pyridin-4-amine |
| J-179 | 2-(4-methoxyphenoxy)-5-(trifluoromethyl)benzenamine |
| J-180 | 6-methylpyridin-2-amine |
| J-181 | 5-chloro-2-fluorobenzenamine |
| J-182 | 1H-indol-4-amine |
| J-183 | 6-morpholinopyridin-3-amine |
| J-184 | aniline |
| J-185 | 1H-indazol-5-amine |
| J-186 | 2-[(Cyclohexyl-methyl-amino)-methyl]-phenylamine |
| J-187 | 2-phenylbenzo[d]oxazol-5-amine |
| J-188 | naphthalen-2-amine |
| J-189 | 2-aminobenzonitrile |
| J-190 | N1,N1-diethyl-3-methylbenzene-1,4-diamine |
| J-191 | aniline |
| J-192 | 2-butylbenzenamine |
| J-193 | 1-(4-aminophenyl)ethanol |
| J-194 | 2-amino-4-methylbenzamide |
| J-195 | quinolin-3-amine |
| J-196 | 2-(piperidin-1-yl)benzenamine |
| J-197 | 3-Amino-benzenesulfonamide |
| J-198 | 2-ethyl-6-methylbenzenamine |
| J-199 | Biphenyl-4-ylamine |
| J-200 | 2-(o-tolyloxy)benzenamine |
| J-201 | 5-amino-3-methylbenzo[d]oxazol-2(3H)-one |
| J-202 | 4-ethylbenzenamine |
| J-203 | 2-isopropylbenzenamine |
| J-204 | 3-(trifluoromethyl)benzenamine |
| J-205 | 2-amino-6-fluorobenzonitrile |
| J-206 | 2-(2-aminophenyl)acetonitrile |
| J-207 | 2-(4-fluorophenoxy)pyridin-3-amine |
| J-208 | aniline |
| J-209 | 2-(4-methylpiperidin-1-yl)benzenamine |
| J-210 | 4-fluorobenzenamine |
| J-211 | 2-propylbenzenamine |
| J-212 | 4-(trifluoromethoxy)benzenamine |
| J-213 | 3-aminophenol |
| J-214 | 2,2-difluorobenzo[d][1,3]dioxol-5-amine |
| J-215 | 2,2,3,3-tetrafluoro-2,3-dihydrobenzo[b][1,4]dioxin-6-amine |
| J-216 | N-(3-aminophenyl)acetamide |
| J-217 | 1-(3-aminophenyl)-3-methyl-1H-pyrazol-5(4H)-one |
| J-218 | 5-(trifluoromethyl)benzene-1,3-diamine |
| J-219 | 5-tert-butyl-2-methoxybenzene-1,3-diamine |
| J-220 | N-(3-amino-4-ethoxyphenyl)acetamide |
| J-221 | N-(3-Amino-phenyl)-methanesulfonamide |
| J-222 | N-(3-aminophenyl)propionamide |
| J-223 | N1,N1-dimethylbenzene-1,3-diamine |
| J-224 | N-(3-amino-4-methoxyphenyl)acetamide |
| J-225 | benzene-1,3-diamine |
| J-226 | 4-methylbenzene-1,3-diamine |
| J-227 | 1H-indol-6-amine |
| J-228 | 6,7,8,9-tetrahydro-5H-carbazol-2-amine |
| J-229 | 1H-indol-6-amine |
| J-230 | 1H-indol-6-amine |
| J-231 | 1H-indol-6-amine |
| J-232 | 1H-indol-6-amine |
| J-233 | 1H-indol-6-amine |
| J-234 | 1H-indol-6-amine |
| J-235 | 1H-indol-6-amine |
| J-236 | 1H-indol-6-amine |
| J-237 | 1H-indol-6-amine |
| J-238 | 1H-indol-6-amine |
| J-239 | 1-(6-Amino-2,3-dihydro-indol-1-yl)-ethanone |
| J-240 | 5-Chloro-benzene-1,3-diamine |

Amides (Compounds of Formula A)

General Scheme:

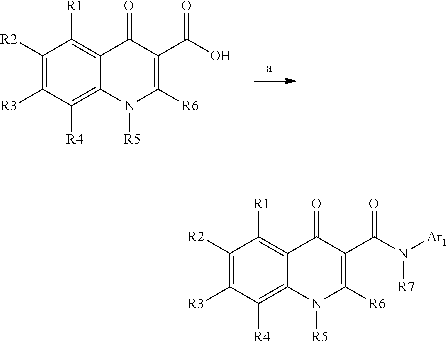

a) Ar₁R7NH, coupling reagent, base, solvent. Examples of conditions used:

HATU, DIEA, DMF; BOP, DIEA, DMF; HBTU, Et₃N, CH₂Cl₂; PFP-TFA, pyridine

### Specific Example

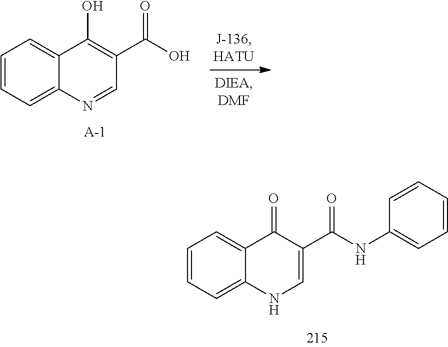

### 215; 4-Oxo-N-phenyl-1H-quinoline-3-carboxamide

To a solution of 4-hydroxy-quinoline-3-carboxylic acid (A-1) (19 mg, 0.1 mmol), HATU (38 mg, 0.1 mmol) and DIEA (34.9 μL, 0.2 mmol) in DMF (1 mL) was added aniline (18.2 μL, 0.2 mmol) and the reaction mixture was stirred at room temperature for 3 h. The resulting solution was filtered and purified by HPLC (10-99% CH₃CN/H₂O) to yield 4-oxo-N-phenyl-1H-quinoline-3-carboxamide (215) (12 mg, 45%). ¹H NMR (400 MHz, DMSO-d₆) δ 12.97 (s, 1H), 12.50 (s, 1H), 8.89 (s, 1H), 8.34 (dd, J=8.1, 1.1 Hz, 1H), 7.83 (t, J=8.3 Hz, 1H), 7.75 (m, 3H), 7.55 (t, J=8.1 Hz, 1H), 7.37 (t, J=7.9 Hz, 2H), 7.10 (t, J=6.8 Hz, 1H); HPLC ret. time 3.02 min, 10-99% CH₃CN, 5 min run; ESI-MS 265.1 m/z (MH⁺).

The table below lists other examples synthesized by the general scheme above.

| Compound of | | |
|---|---|---|
| Formula A | Acid | Amine |
| 2 | A-1 | C-2 |
| 3 | A-1 | J-17 |
| 4 | A-1 | J-110 |
| 5 | A-1 | G-2 |
| 6 | A-1 | E-8 |
| 7 | A-1 | J-118 |
| 8 | A-1 | D-7 |
| 9 | A-1 | J-197 |
| 11 | A-1 | F-7 |
| 12 | A-1 | F-6 |
| 13 | A-1 | E-2 |
| 15 | A-1 | J-56 |
| 16 | A-1 | J-211 |
| 18 | A-1 | J-161 |
| 19 | A-1 | J-112 |
| 20 | A-1 | J-200 |
| 21 | A-1 | J-98 |
| 23 | A-1 | C-15 |
| 24 | A-1 | J-72 |
| 25 | A-1 | F-57 |
| 26 | A-1 | J-196 |
| 29 | A-21 | J-208 |
| 31 | A-1 | J-87 |
| 32 | A-1 | B-21 |
| 33 | A-1 | J-227 |
| 34 | A-1 | C-19 |
| 36 | A-1 | J-203 |
| 37 | A-1 | J-80 |
| 38 | A-1 | J-46 |
| 39 | A-17 | D-10 |
| 40 | A-1 | J-125 |
| 42 | A-1 | J-95 |
| 43 | A-1 | C-16 |
| 44 | A-1 | J-140 |
| 45 | A-1 | J-205 |
| 47 | A-1 | J-102 |
| 48 | A-1 | J-181 |
| 49 | A-1 | F-25 |
| 50 | A-1 | J-19 |
| 51 | A-7 | B-24 |
| 52 | A-1 | F-2 |
| 53 | A-1 | J-178 |
| 54 | A-1 | J-26 |
| 55 | A-1 | J-219 |
| 56 | A-1 | J-74 |
| 57 | A-1 | J-61 |
| 58 | A-1 | D-4 |
| 59 | A-1 | F-35 |
| 60 | A-1 | D-11 |
| 61 | A-1 | J-174 |
| 62 | A-1 | J-106 |
| 63 | A-1 | F-47 |
| 64 | A-1 | J-111 |
| 66 | A-1 | J-214 |
| 67 | A-10 | J-236 |
| 68 | A-1 | F-55 |
| 69 | A-1 | D-8 |
| 70 | A-1 | F-11 |
| 71 | A-1 | F-61 |
| 72 | A-1 | J-66 |
| 73 | A-1 | J-157 |
| 74 | A-1 | J-104 |
| 75 | A-1 | J-195 |
| 76 | A-1 | F-46 |
| 77 | A-1 | B-20 |
| 78 | A-1 | J-92 |
| 79 | A-1 | F-41 |
| 80 | A-1 | J-30 |
| 81 | A-1 | J-222 |
| 82 | A-1 | J-190 |
| 83 | A-1 | F-40 |
| 84 | A-1 | J-32 |
| 85 | A-1 | F-53 |
| 86 | A-1 | J-15 |
| 87 | A-1 | J-39 |
| 88 | A-1 | G-3 |
| 89 | A-1 | J-134 |
| 90 | A-1 | J-18 |
| 91 | A-1 | J-38 |
| 92 | A-1 | C-13 |
| 93 | A-1 | F-68 |
| 95 | A-1 | J-189 |
| 96 | A-1 | B-9 |
| 97 | A-1 | F-34 |
| 99 | A-1 | J-4 |
| 100 | A-1 | J-182 |
| 102 | A-1 | J-117 |
| 103 | A-2 | C-9 |
| 104 | A-1 | B-4 |
| 106 | A-1 | J-11 |
| 107 | A-1 | DC-6 |
| 108 | A-1 | DC-3 |
| 109 | A-1 | DC-4 |
| 110 | A-1 | J-84 |
| 111 | A-1 | J-43 |
| 112 | A-11 | J-235 |
| 113 | A-1 | B-7 |
| 114 | A-1 | D-18 |
| 115 | A-1 | F-62 |
| 116 | A-3 | J-229 |
| 118 | A-1 | F-12 |
| 120 | A-1 | J-1 |
| 121 | A-1 | J-130 |
| 122 | A-1 | J-49 |
| 123 | A-1 | F-66 |
| 124 | A-2 | B-24 |
| 125 | A-1 | J-143 |
| 126 | A-1 | C-25 |
| 128 | A-22 | J-176 |
| 130 | A-14 | J-233 |
| 131 | A-1 | J-240 |
| 132 | A-1 | J-220 |
| 134 | A-1 | F-58 |
| 135 | A-1 | F-19 |
| 136 | A-1 | C-8 |
| 137 | A-6 | C-9 |
| 138 | A-1 | F-44 |
| 139 | A-1 | F-59 |
| 140 | A-1 | J-64 |
| 142 | A-1 | J-10 |
| 143 | A-1 | C-7 |
| 144 | A-1 | J-213 |
| 145 | A-1 | B-18 |
| 146 | A-1 | J-55 |
| 147 | A-1 | J-207 |
| 150 | A-1 | J-162 |
| 151 | A-1 | F-67 |
| 152 | A-1 | J-156 |
| 153 | A-1 | C-23 |
| 154 | A-1 | J-107 |
| 155 | A-1 | J-3 |
| 156 | A-1 | F-36 |
| 160 | A-1 | D-6 |
| 161 | A-1 | C-3 |
| 162 | A-1 | J-171 |
| 164 | A-1 | J-204 |
| 165 | A-1 | J-65 |
| 166 | A-1 | F-54 |
| 167 | A-1 | J-226 |
| 168 | A-1 | J-48 |
| 169 | A-1 | B-1 |
| 170 | A-1 | J-42 |
| 171 | A-1 | F-52 |
| 172 | A-1 | F-64 |
| 173 | A-1 | J-180 |
| 174 | A-1 | F-63 |
| 175 | A-1 | DC-2 |
| 176 | A-1 | J-212 |
| 177 | A-1 | J-57 |
| 178 | A-1 | J-153 |
| 179 | A-1 | J-154 |
| 180 | A-1 | J-198 |
| 181 | A-1 | F-1 |
| 182 | A-1 | F-37 |
| 183 | A-1 | DC-1 |
| 184 | A-15 | J-231 |
| 185 | A-1 | J-173 |
| 186 | A-1 | B-15 |
| 187 | A-1 | B-3 |
| 188 | A-1 | B-25 |
| 189 | A-1 | J-24 |
| 190 | A-1 | F-49 |
| 191 | A-1 | J-23 |
| 192 | A-1 | J-36 |
| 193 | A-1 | J-68 |
| 194 | A-1 | J-37 |
| 195 | A-1 | J-127 |
| 197 | A-1 | J-167 |
| 198 | A-1 | J-210 |
| 199 | A-1 | F-3 |
| 200 | A-1 | H-1 |
| 201 | A-1 | J-96 |
| 202 | A-1 | F-28 |
| 203 | A-1 | B-2 |
| 204 | A-1 | C-5 |
| 205 | A-1 | J-179 |
| 206 | A-1 | J-8 |
| 207 | A-1 | B-17 |
| 208 | A-1 | C-12 |
| 209 | A-1 | J-126 |
| 210 | A-17 | J-101 |
| 211 | A-1 | J-152 |
| 212 | A-1 | J-217 |
| 213 | A-1 | F-51 |
| 214 | A-1 | J-221 |
| 215 | A-1 | J-136 |
| 216 | A-1 | J-147 |
| 217 | A-1 | J-185 |
| 218 | A-2 | C-13 |
| 219 | A-1 | J-114 |
| 220 | A-1 | C-26 |
| 222 | A-1 | J-35 |
| 223 | A-1 | F-23 |
| 224 | A-1 | I-1 |
| 226 | A-1 | J-129 |
| 227 | A-1 | J-120 |
| 228 | A-1 | J-169 |
| 229 | A-1 | J-59 |
| 230 | A-1 | J-145 |
| 231 | A-1 | C-17 |
| 233 | A-1 | J-239 |
| 234 | A-1 | B-22 |
| 235 | A-1 | E-9 |
| 236 | A-1 | J-109 |
| 240 | A-1 | J-34 |
| 241 | A-1 | J-82 |
| 242 | A-1 | D-2 |
| 244 | A-1 | J-228 |
| 245 | A-1 | J-177 |
| 246 | A-1 | J-78 |
| 247 | A-1 | F-33 |
| 250 | A-1 | J-224 |
| 252 | A-1 | J-135 |
| 253 | A-1 | F-30 |
| 254 | A-2 | B-20 |
| 255 | A-8 | C-9 |
| 256 | A-1 | J-45 |
| 257 | A-1 | J-67 |
| 259 | A-1 | B-14 |
| 261 | A-1 | F-13 |
| 262 | A-1 | DC-7 |
| 263 | A-1 | J-163 |
| 264 | A-1 | J-122 |
| 265 | A-1 | J-40 |
| 266 | A-1 | C-14 |
| 267 | A-1 | J-7 |
| 268 | A-1 | E-7 |
| 270 | A-1 | B-5 |
| 271 | A-1 | D-9 |
| 273 | A-1 | H-2 |
| 274 | A-8 | B-24 |
| 276 | A-1 | J-139 |
| 277 | A-1 | F-38 |
| 278 | A-1 | F-10 |
| 279 | A-1 | F-56 |
| 280 | A-1 | J-146 |
| 281 | A-1 | J-62 |
| 283 | A-1 | F-18 |
| 284 | A-1 | J-16 |
| 285 | A-1 | F-45 |
| 286 | A-1 | J-119 |
| 287 | A-3 | C-13 |
| 288 | A-1 | C-6 |
| 289 | A-1 | J-142 |
| 290 | A-1 | F-15 |
| 291 | A-1 | C-10 |
| 292 | A-1 | J-76 |
| 293 | A-1 | J-144 |
| 294 | A-1 | J-54 |
| 295 | A-1 | J-128 |
| 296 | A-17 | J-12 |
| 297 | A-1 | J-138 |
| 301 | A-1 | J-14 |
| 302 | A-1 | F-5 |
| 303 | A-1 | J-13 |
| 304 | A-1 | E-1 |
| 305 | A-1 | F-17 |
| 306 | A-1 | F-20 |
| 307 | A-1 | F-43 |
| 308 | A-1 | J-206 |
| 309 | A-1 | J-5 |
| 310 | A-1 | J-70 |
| 311 | A-1 | J-60 |
| 312 | A-1 | F-27 |
| 313 | A-1 | F-39 |
| 314 | A-1 | J-116 |
| 315 | A-1 | J-58 |
| 317 | A-1 | J-85 |
| 319 | A-2 | C-7 |
| 320 | A-1 | B-6 |
| 321 | A-1 | J-44 |
| 322 | A-1 | J-22 |
| 324 | A-1 | J-172 |
| 325 | A-1 | J-103 |
| 326 | A-1 | F-60 |
| 328 | A-1 | J-115 |
| 329 | A-1 | J-148 |
| 330 | A-1 | J-133 |
| 331 | A-1 | J-105 |
| 332 | A-1 | J-9 |
| 333 | A-1 | F-8 |
| 334 | A-1 | DC-5 |
| 335 | A-1 | J-194 |
| 336 | A-1 | J-192 |
| 337 | A-1 | C-24 |
| 338 | A-1 | J-113 |
| 339 | A-1 | B-8 |
| 344 | A-1 | F-22 |
| 345 | A-2 | J-234 |
| 346 | A-12 | J-6 |
| 348 | A-1 | F-21 |
| 349 | A-1 | J-29 |
| 350 | A-1 | J-100 |
| 351 | A-1 | B-23 |
| 352 | A-1 | B-10 |
| 353 | A-1 | D-10 |
| 354 | A-1 | J-186 |
| 355 | A-1 | J-25 |
| 357 | A-1 | B-13 |
| 358 | A-24 | J-232 |
| 360 | A-1 | J-151 |
| 361 | A-1 | F-26 |
| 362 | A-1 | J-91 |
| 363 | A-1 | F-32 |
| 364 | A-1 | J-88 |
| 365 | A-1 | J-93 |
| 366 | A-1 | F-16 |
| 367 | A-1 | F-50 |
| 368 | A-1 | D-5 |
| 369 | A-1 | J-141 |
| 370 | A-1 | J-90 |
| 371 | A-1 | J-79 |
| 372 | A-1 | J-209 |
| 373 | A-1 | J-21 |
| 374 | A-16 | J-238 |
| 375 | A-1 | J-71 |
| 376 | A-1 | J-187 |
| 377 | A-5 | J-237 |
| 378 | A-1 | D-3 |
| 380 | A-1 | J-99 |
| 381 | A-1 | B-24 |
| 383 | A-1 | B-12 |
| 384 | A-1 | F-48 |
| 385 | A-1 | J-83 |
| 387 | A-1 | J-168 |
| 388 | A-1 | F-29 |
| 389 | A-1 | J-27 |
| 391 | A-1 | F-9 |
| 392 | A-1 | J-52 |
| 394 | A-22 | J-170 |
| 395 | A-1 | C-20 |
| 397 | A-1 | J-199 |
| 398 | A-1 | J-77 |
| 400 | A-1 | J-183 |
| 401 | A-1 | F-4 |
| 402 | A-1 | J-149 |
| 403 | A-1 | C-22 |
| 405 | A-1 | J-33 |
| 406 | A-6 | B-24 |
| 407 | A-3 | C-7 |
| 408 | A-1 | J-81 |
| 410 | A-1 | F-31 |
| 411 | A-13 | J-191 |
| 412 | A-1 | B-19 |
| 413 | A-1 | J-131 |
| 414 | A-1 | J-50 |
| 417 | A-1 | F-65 |
| 418 | A-1 | J-223 |
| 419 | A-1 | J-216 |
| 420 | A-1 | G-1 |
| 421 | A-1 | C-18 |
| 422 | A-1 | J-20 |
| 423 | A-1 | B-16 |
| 424 | A-1 | F-42 |
| 425 | A-1 | J-28 |
| 426 | A-1 | C-11 |
| 427 | A-1 | J-124 |
| 428 | A-1 | C-1 |
| 429 | A-1 | J-218 |
| 430 | A-1 | J-123 |
| 431 | A-1 | J-225 |
| 432 | A-1 | F-14 |
| 433 | A-1 | C-9 |
| 434 | A-1 | J-159 |
| 435 | A-1 | J-41 |
| 436 | A-1 | F-24 |
| 437 | A-1 | J-75 |
| 438 | A-1 | E-10 |
| 439 | A-1 | J-164 |
| 440 | A-1 | J-215 |
| 441 | A-1 | D-19 |
| 442 | A-1 | J-165 |
| 443 | A-1 | J-166 |
| 444 | A-1 | E-6 |
| 445 | A-1 | J-97 |
| 446 | A-1 | J-121 |
| 447 | A-1 | J-51 |
| 448 | A-1 | J-69 |
| 449 | A-1 | J-94 |
| 450 | A-1 | J-193 |
| 451 | A-1 | J-31 |
| 452 | A-1 | J-108 |
| 453 | A-1 | D-1 |
| 454 | A-1 | J-47 |
| 455 | A-1 | J-73 |
| 456 | A-1 | J-137 |
| 457 | A-1 | J-155 |
| 458 | A-1 | C-4 |
| 459 | A-1 | J-53 |
| 461 | A-1 | J-150 |
| 463 | A-1 | J-202 |
| 464 | A-3 | C-9 |
| 465 | A-1 | E-4 |
| 466 | A-1 | J-2 |
| 467 | A-1 | J-86 |
| 468 | A-20 | J-184 |
| 469 | A-12 | J-132 |
| 470 | A-1 | J-160 |
| 473 | A-21 | J-89 |
| 474 | A-1 | J-201 |
| 475 | A-1 | J-158 |
| 477 | A-1 | J-63 |
| 478 | A-1 | B-11 |
| 479 | A-4 | J-230 |
| 480 | A-23 | J-175 |
| 481 | A-1 | J-188 |
| 483 | A-1 | C-21 |
| 484 | A-1 | D-14 |
| B-26-I | A-1 | B-26 |
| B-27-I | A-1 | B-27 |
| C-27-I | A-1 | C-27 |
| D-12-I | A-1 | D-12 |
| D-13-I | A-1 | D-13 |
| D-15-I | A-1 | D-15 |
| D-16-I | A-1 | D-16 |
| D-17-I | A-1 | D-17 |
| DC-10-I | A-1 | DC-10 |
| DC-8-I | A-1 | DC-8 |
| DC-9-I | A-1 | DC-9 |

### Indoles

### Example 1

### General Scheme

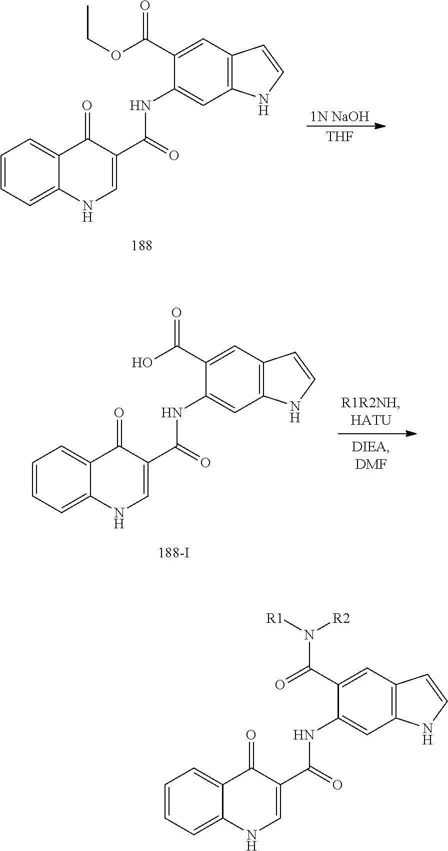

### Specific Example

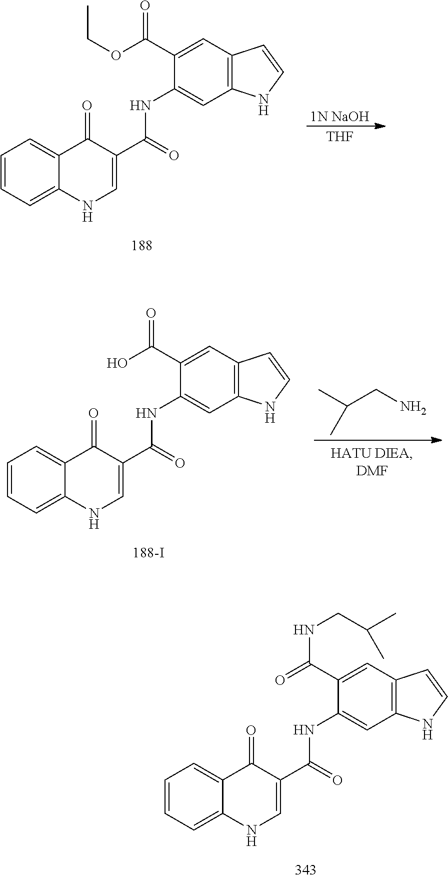

### 188-I; 6-[(4-Oxo-1H-quinolin-3-yl)carbonylamino]-1H-indole-5-carboxylic acid

A mixture of 6-[(4-oxo-1H-quinolin-3-yl)carbonylamino]-1H-indole-5-carboxylic acid ethyl ester (188) (450 mg, 1.2 mmol) and 1N NaOH solution (5 mL) in THF (10 mL) was heated at 85° C. overnight. The reaction mixture was partitioned between EtOAc and water. The aqueous layer was acidified with 1N HCl solution to pH 5, and the precipitate was filtered, washed with water and air dried to yield 6-[(4-oxo-1H-quinolin-3-yl)carbonylamino]-1H-indole-5-carboxylic acid (188-I) (386 mg, 93%). ¹H-NMR (400 MHz, DMSO-d₆) δ 12.92-12.75 (m, 2H), 11.33 (s, 1H), 8.84 (s, 1H), 8.71 (s, 1H), 8.30 (dd, J=8.1, 0.9 Hz, 1H), 8.22 (s, 1H), 7.80-7.72 (m, 2H), 7.49 (t, J=8.0 Hz, 1H), 7.41 (t, J=2.7 Hz, 1H), 6.51 (m, 1H); HPLC ret. time 2.95 min, 10-99% CH₃CN, 5 min run; ESI-MS 376.2 m/z (MH⁺).

### 343; N-[5-(Isobutylcarbamoyl)-1H-indol-6-yl]-4-oxo-1H-quinoline-3-carboxamide

To a solution of 6-[(4-oxo-1H-quinolin-3-yl)carbonylamino]-1H-indole-5-carboxylic acid (188-I) (26 mg, 0.08 mmol), HATU (38 mg, 0.1 mmol) and DIEA (35 μL, 0.2 mmol) in DMF (1 mL) was added isobutylamine (7 mg, 0.1 mmol) and the reaction mixture was stirred at 65° C. overnight. The resulting solution was filtered and purified by HPLC (10-99% CH₃CN/H₂O) to yield the product, N-[5-(isobutylcarbamoyl)-1H-indol-6-yl]-4-oxo-1H-quinoline-3-carboxamide (343) (20 mg, 66%). ¹H-NMR (400 MHz, DMSO-d₆) δ 12.66 (d, J=7.4 Hz, 1H), 12.42 (s, 1H), 11.21 (s, 1H), 8.81 (d, J=6.6 Hz, 1H), 8.47 (s, 1H), 8.36 (t, J=5.6 Hz, 1H), 8.30 (d, J=8.4 Hz, 1H), 7.79 (t, J=7.9 Hz, 1H), 7.72-7.71 (m, 2H), 7.51 (t, J=7.2 Hz, 1H), 7.38 (m, 1H), 6.48 (m, 1H), 3.10 (t, J=6.2 Hz, 2H), 1.88 (m, 1H), 0.92 (d, J=6.7 Hz, 6H); HPLC ret. time 2.73 min, 10-99% CH₃CN, 5 min run; ESI-MS 403.3 m/z (MH⁺).

### Another Example

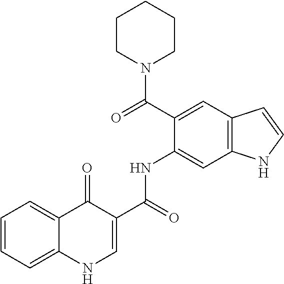 [SMILES: O=C(Nc1cc2[nH]ccc2cc1C(=O)N1CCCCC1)c1c[nH]c2ccccc2c1=O]

### 148; 4-Oxo-N-[5-(1-piperidylcarbonyl)-1H-indol-6-yl]-1H-quinoline-3-carboxamide

4-Oxo-N-[5-(1-piperidylcarbonyl)-1H-indol-6-yl]-1H-quinoline-3-carboxamide (148) was synthesized following the general scheme above, coupling the acid (188-I) with piperidine. Overall yield (12%). HPLC ret. time 2.79 min, 10-99% CH₃CN, 5 min run; ESI-MS 415.5 m/z (MH⁺).

### Example 2

### General Scheme

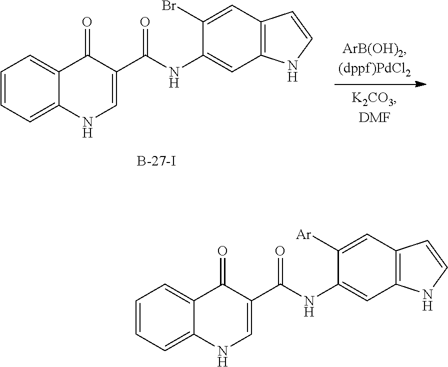

### Specific Example

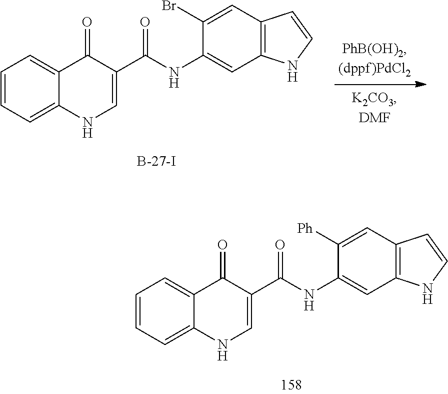 [SMILES: O=C(Nc1cc2[nH]ccc2cc1Br)c1c[nH]c2ccccc2c1=O ; O=C(Nc1cc2[nH]ccc2cc1-c1ccccc1)c1c[nH]c2ccccc2c1=O]

### 158; 4-Oxo-N-(5-phenyl-4H-indol-6-yl)-1H-quinoline-3-carboxamide

A mixture of N-(5-bromo-1H-indol-6-yl)-4-oxo-1H-quinoline-3-carboxamide (B-274) (38 mg, 0.1 mol), phenyl boronic acid (18 mg, 0.15 mmol), (dppf)PdCl₂ (cat.), and K₂CO₃ (100 μL, 2M solution) in DMF (1 mL) was heated in the microwave at 180° C. for 10 min. The reaction was filtered and purified by HPLC (10-99% CH₃CN/H₂O) to yield the product, 4-oxo-N-(5-phenyl-1H-indol-6-yl)-1H-quinoline-3-carboxamide (158) (5 mg, 13%). HPLC ret. time 3.05 min, 10-99% CH₃CN, 5 min run; ESI-MS 380.2 m/z (MH⁺).

The table below lists other examples synthesized following the general scheme above.

| Compound of | |
|---|---|
| formula I | Boronic acid |
| 237 | 2-methoxyphenylboronic acid |
| 327 | 2-ethoxyphenylboronic acid |
| 404 | 2,6-dimethoxyphenylboronic acid |
| 1 | 5-chloro-2-methoxy-phenylboronic acid |
| 342 | 4-isopropylphenylboronic acid |
| 347 | 4-(2-Dimethylaminoethylcarbamoyl)phenylboronic acid |
| 65 | 3-pyridinylboronic acid |

### Example 3

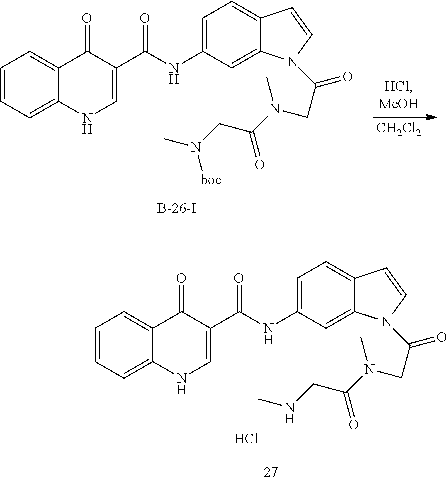

### 27; N-[1-[2-[Methyl-(2-methylaminoacetyl)-amino]acetyl]-1H-indol-6-yl]-4-oxo-1H-quinoline-3-carboxamide

To a solution of methyl-{[methyl-(2-oxo-2-{6-[(4-oxo-1,4-dihydro-quinoline-3-carbonyl)-amino]-indol-1-yl}-ethyl)-carbamoyl]-methyl}-carbamic acid tert-butyl ester (B-26-I) (2.0 g, 3.7 mmol) dissolved in a mixture of CH₂Cl₂ (50 mL) and methanol (15 mL) was added HCl solution (60 mL, 1.25 M in methanol). The reaction was stirred at room temperature for 64 h. The precipitated product was collected via filtration, washed with diethyl ether and dried under high vacuum to provide the HCl salt of the product, N-[1-[2-[methyl-(2-methylaminoacetyl)-amino]acetyl]-1H-indol-6-yl]-4-oxo-1H-quinoline-3-carboxamide (27) as a greyish white solid (1.25 g, 70%). ¹H-NMR (400 MHz, DMSO-d6) δ 13.20 (d, J=6.7 Hz, 1H), 12.68 (s, 1H), 8.96-8.85 (m, 1H), 8.35 (d, J=7.9 Hz, 1H), 7.91-7.77 (m, 3H), 7.64-7.54 (m, 3H), 6.82 (m, 1H), 5.05 (s, 0.7H), 4.96 (s, 1.3H), 4.25 (t, J=5.6 Hz, 1.3H), 4.00 (t, J=5.7 Hz, 0.7H), 3.14 (s, 2H), 3.02 (s, 1H), 2.62 (t, J=5.2 Hz, 2H), 2.54 (t, J=5.4 Hz, 1H); HPLC ret. time 2.36 min, 10-99% CH₃CN, 5 min run; ESI-MS 446.5 m/z (MH⁺).

### Phenols

### Example 1

### General Scheme

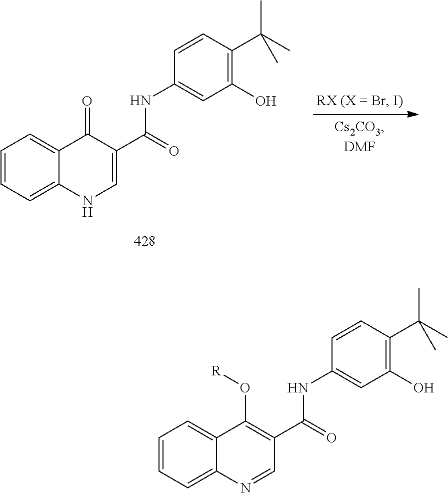

### Specific Example

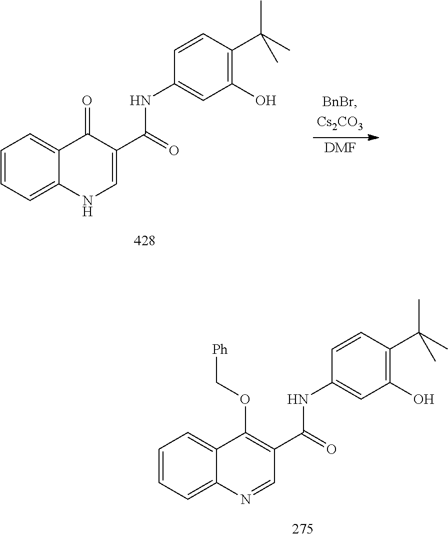

### 275; 4-Benzyloxy-N-(3-hydroxy-4-tert-butyl-phenyl)-quinoline-3-carboxamide

To a mixture of N-(3-hydroxy-4-tert-butyl-phenyl)-4-oxo-1H-quinoline-3-carboxamide (428) (6.7 mg, 0.02 mmol) and Cs₂CO₃ (13 mg, 0.04 mmol) in DMF (0.2 mL) was added BnBr (10 μL, 0.08 mmol). The reaction mixture was stirred at room temperature for 3 h. The reaction mixture was filtered and purified using HPLC to give 4-benzyloxy-N-(3-hydroxy-4-tert-butyl-phenyl)-quinoline-3-carboxamide (275). ¹H NMR (400 MHz, DMSO-d₆) δ 12.23 (s, 1H), 9.47 (s, 1H), 9.20 (s, 1H), 8.43 (d, J=7.9 Hz, 1H), 7.79 (t, J=2.0 Hz, 2H), 7.56 (m, 1H), 7.38-7.26 (m, 6H), 7.11 (d, J=8.4 Hz, 1H), 6.99 (dd, J=8.4, 2.1 Hz, 1H), 5.85 (s, 2H), 1.35 (s, 9H). HPLC ret. time 3.93 min, 10-99% CH₃CN, 5 min run; ESI-MS 427.1 m/z (MH⁺).

### Another Example

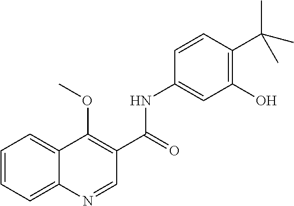 [SMILES: COc1c(C(=O)Nc2ccc(C(C)(C)C)c(O)c2)cnc2ccccc12]

### 415; N-(3-Hydroxy-4-tert-butyl-phenyl)-4-methoxy-quinoline-3-carboxamide

N-(3-Hydroxy-4-tert-butyl-phenyl)-4-methoxy-quinoline-3-carboxamide (415) was synthesized following the general scheme above reacting N-(3-hydroxy-4-tert-butyl-phenyl)-4-oxo-1H-quinoline-3-carboxamide (428) with methyl iodide. ¹H NMR (400 MHz, DMSO-d₆) δ 12.26 (s, 1H), 9.46 (s, 1H), 8.99 (s, 1H), 8.42 (t, J=4.2 Hz, 1H), 7.95-7.88 (m, 2H), 7.61-7.69 (m, 1H), 7.38 (d, J=2.1 Hz, 1H), 7.10 (d, J=8.4 Hz, 1H), 6.96 (dd, J=8.4, 2.1 Hz, 1H), 4.08 (s, 3H), 1.35 (s, 9H); HPLC ret. time 3.46 min, 10-99% CH₃CN, 5 min run; ESI-MS 351.5 m/z (MH⁺).

### Example 2

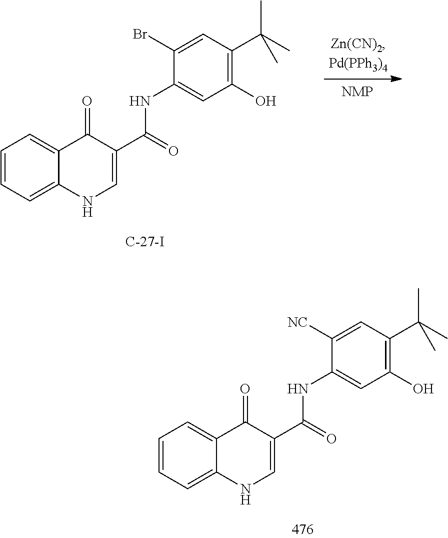 [SMILES: CC(C)(C)c1cc(Br)c(NC(=O)c2c[nH]c3ccccc3c2=O)cc1O ; CC(C)(C)c1cc(C#N)c(NC(=O)c2c[nH]c3ccccc3c2=O)cc1O]

### 476; N-(4-tert-Butyl-2-cyano-5-hydroxyphenyl)-1,4-dihydro-4-oxoquinoline-3-carboxamide

To a suspension of N-(4-tert-butyl-2-bromo-5-hydroxyphenyl)-1,4-dihydro-4-oxoquinoline-3-carboxamide (C-27-I) (84 mg, 0.2 mmol), Zn(CN)₂ (14 mg, 0.12 mmol) in NMP (1 mL) was added Pd(PPh₃)₄ (16 mg, 0.014 mmol) under nitrogen. The mixture was heated in a microwave oven at 200° C. for 1 h, filtered and purified using prepative HPLC to give N-(4-tert-butyl-2-cyano-5-hydroxyphenyl)-1,4-dihydro-4-oxoquinoline-3-carboxamide (476). ¹H NMR (400 MHz, DMSO-d₆) δ 13.00 (d, J=6.4 Hz, 1H), 12.91 (s, 1H), 10.72 (s, 1H), 8.89 (d, J=6.8 Hz, 1H), 8.34 (d, J=8.2 Hz, 1H), 8.16 (s, 1H), 7.85-7.75 (m, 2H), 7.56-7.54 (m, 1H), 7.44 (s, 1H), 1.35 (s, 9H); HPLC ret. time 3.42 min, 10-100% CH₃CN, 5 min gradient; ESI-MS 362.1 m/z (MH⁺).

### Anilines

### Example 1

### General Scheme

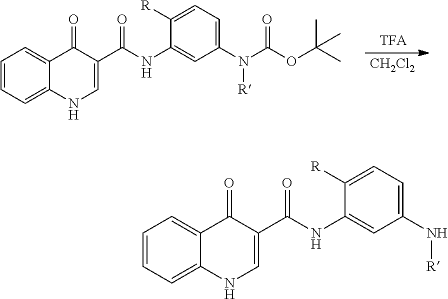

### Specific Example

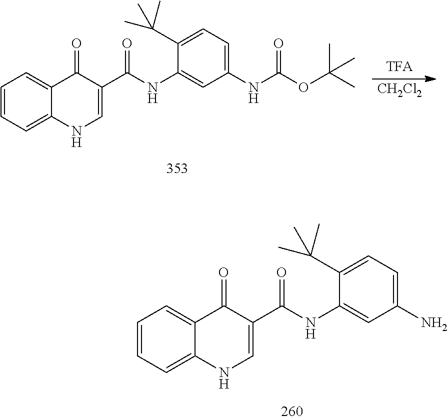

### 260; N-(5-Amino-2-tert-butyl-phenyl)-4-oxo-1H-quinoline-3-carboxamide

A mixture of [3-[(4-oxo-1H-quinolin-3-yl)carbonylamino]-4-tert-butyl-phenyl]aminoformic acid tert-butyl ester (353) (33 mg, 0.08 mmol), TFA (1 mL) and CH₂Cl₂ (1 mL) was stirred at room temperature overnight. The solution was concentrated and the residue was dissolved in DMSO (1 mL) and purified by HPLC (10-99% CH₃CN/H₂O) to yield the product, N-(5-amino-2-tert-butyl-phenyl)-4-oxo-1H-quinoline-3-carboxamide (260) (15 mg, 56%). ¹H NMR (400 MHz, DMSO-d₆) δ 13.23 (d, J=6.6 Hz, 1H), 12.20 (s, 1H), 10.22 (br s, 2H), 8.88 (d, J=6.8 Hz, 1H), 8.34 (d, J=7.8 Hz, 1H), 7.86-7.80 (m, 3H), 7.56-7.52 (m, 2H), 7.15 (dd, J=8.5, 2.4 Hz, 1H), 1.46 (s, 9H); HPLC ret. time 2.33 min, 10-99% CH₃CN, 5 min run; ESI-MS 336.3 m/z (MH⁺).

The table below lists other examples synthesized following the general scheme above.

| Starting | |
|---|---|
| Intermediate | Product |
| 60 | 101 |
| D-12-I | 282 |
| D-13-I | 41 |
| 114 | 393 |
| D-16-I | 157 |
| D-15-I | 356 |
| D-17-I | 399 |

### Example 2

### General Scheme

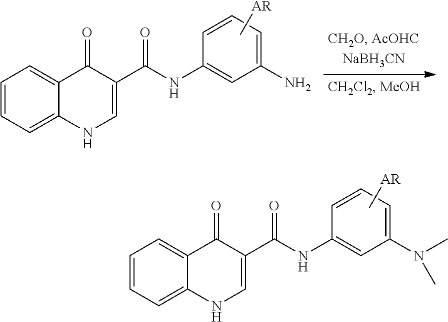

### Specific Example

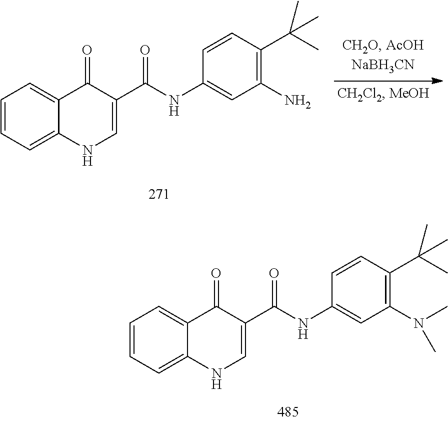 [SMILES: CC(C)(C)c1ccc(NC(=O)c2c[nH]c3ccccc3c2=O)cc1N ; CN(C)c1cc(NC(=O)c2c[nH]c3ccccc3c2=O)ccc1C(C)(C)C]

### 485; N-(3-Dimethylamino-4-tert-butyl-phenyl)-4-oxo-1H-quinoline-3-carboxamide

To a suspension of N-(3-amino-4-tert-butyl-phenyl)-4-oxo-1H-quinoline-3-carboxamide (271) (600 mg, 1.8 mmol) in CH₂Cl₂ (15 mL) and methanol (5 mL) were added acetic acid (250 μL) and formaldehyde (268 μL, 3.6 mmol, 37 wt % in water). After 10 min, sodium cyanoborohydride (407 mg, 6.5 mmol) was added in one portion. Additional formaldehyde (135 μL, 1.8 mmol, 37 wt % in water) was added at 1.5 and 4.2 h. After 4.7 h, the mixture was diluted with ether (40 mL), washed with water (25 mL) and brine (25 mL), dried (Na₂SO₄), filtered, and concentrated. The resulting red-brown foam was purified by preparative HPLC to afford N-(3-dimethylamino-4-tert-butyl-phenyl)-4-oxo-1H-quinoline-3-carboxamide (485) (108 mg, 17%). ¹H NMR (300 MHz, CDCl₃) δ 13.13 (br s, 1H), 12.78 (s, 1H), 8.91 (br s, 1H), 8.42 (br s, 1H), 8.37 (4, J=8.1 Hz, 1H), 7.72-7.58 (m, 2H), 7.47-7.31 (m, 3H), 3.34 (s, 6H), 1.46 (s, 9H); HPLC ret. time 2.15 min, 10-100% CH₃CN, 5 min run; ESI-MS 364.3 m/z (MH⁺).

The table below lists other examples synthesized following the general scheme above.

| Starting | |
|---|---|
| Intermediate | Product |
| 69 | 117 |
| 160 | 462 |
| 282 | 409 |
| 41 | 98 |

### Example 3

### General Scheme

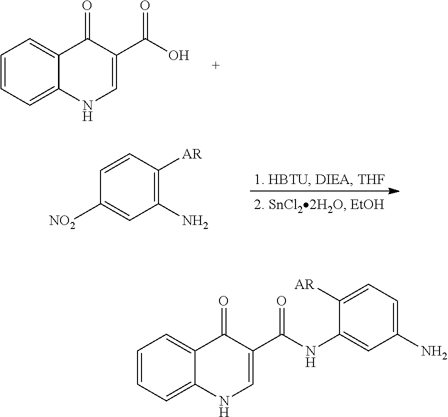

### Specific Example

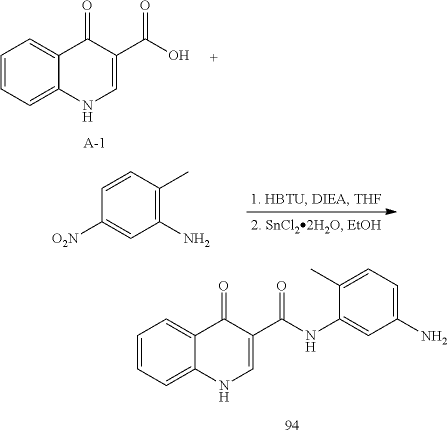

### 94; N-(5-Amino-2-methyl-phenyl)-4-oxo-1H-quinoline-3-carboxamide

To a solution of 4-hydroxy-quinoline-3-carboxylic acid (A-1) (50 mg, 0.26 mmol), HBTU (99 mg, 0.26 mmol) and DIEA (138 μL, 0.79 mmol) in THF (2.6 mL) was added 2-methyl-5-nitro-phenylamine (40 mg, 0.26 mmol). The mixture was heated at 150° C. in the microwave for 20 min and the resulting solution was concentrated. The residue was dissolved in EtOH (2 mL) and SnCl₂.2H₂O (293 mg, 1.3 mmol) was added. The reaction was stirred at room temperature overnight. The reaction mixture was basified with sat. NaHCO₃ solution to pH 7-8 and extracted with ethyl acetate. The combined organic layers were washed with brine, dried over Na₂SO₄, filtered and concentrated. The residue was dissolved in DMSO and purified by HPLC (10-99% CH₃CN/H₂O) to yield the product, N-(5-amino-2-methyl-phenyl)-4-oxo-1H-quinoline-3-carboxamide (94) (6 mg, 8%). HPLC ret. time 2.06 min, 10-99% CH₃CN, 5 min run; ESI-MS 294.2 m/z (MH⁺).

### Another Example

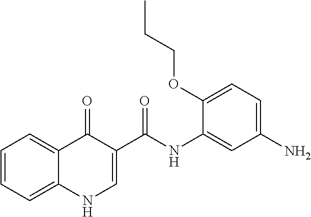 [SMILES: CCCOc1ccc(N)cc1NC(=O)c1c[nH]c2ccccc2c1=O]

### 17; N-(5-Amino-2-propoxy-phenyl)-4-oxo-1H-quinoline-3-carboxamide

N-(5-Amino-2-propoxy-phenyl)-4-oxo-1H-quinoline-3-carboxamide (17) was made following the general scheme above starting from 4-hydroxy-quinoline-3-carboxylic acid (A-1) and 5-nitro-2-propoxy-phenylamine. Yield (9%). HPLC ret. time 3.74 min, 10-99% CH₃CN, 5 min run; ESI-MS 338.3 m/z (MH⁺).

### Example 4

### General Scheme

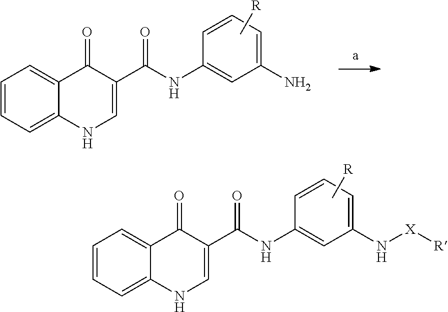

X═CO, CO₂, SO₂: a) R2XCl, DIEA, THF or R2XCl, NMM, 1,4-dioxane or R2XCl, Et₃N, CH₂Cl₂, DMF.

### Specific Example

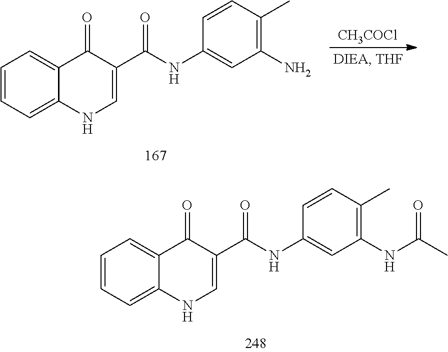

### 248; N-(3-Acetylamino-4-methyl-phenyl)-4-oxo-1H-quinoline-3-carboxamide

To a solution of N-(3-amino-4-methyl-phenyl)-4-oxo-1H-quinoline-3-carboxamide (167) (33 mg, 0.11 mmol) and DIEA (49 μL, 0.28 mmol) in THF (1 mL) was added acetyl chloride (16 μL, 0.22 mmol). The reaction was stirred at room temperature for 30 min. LCMS analysis indicated that diacylation had occurred. A solution of piperidine (81 μl., 0.82 mmol) in CH₂Cl₂ (2 mL) was added and the reaction stirred for a further 30 min at which time only the desired product was detected by LCMS. The reaction solution was concentrated and the residue was dissolved in DMSO and purified by HPLC (10-99% CH₃CN/H₂O) to yield the product, N-(3-acetylamino-4-methyl-phenyl)-4-oxo-1H-quinoline-3-carboxamide (248) (4 mg, 11%). ¹H NMR (400 MHz, DMSO-d₆) δ 12.95 (d, J=6.6 Hz, 1H), 12.42 (s, 1H), 9.30 (s, 1H), 8.86 (d, J=6.8 Hz, 1H), 8.33 (dd, J=8.1, 1.3 Hz, 1H), 7.85-7.81 (m, 2H), 7.76 (d, J=7.8 Hz, 1H), 7.55 (t, J=8.1 Hz, 1H), 7.49 (dd, J=8.2, 2.2 Hz, 1H), 7.18 (d, J=8.3 Hz, 1H), 2.18 (s, 3H), 2.08 (s, 3H); HPLC ret. time 2.46 min, 10-99% CH₃CN, 5 min run; ESI-MS 336.3 m/z (MH⁺).

The table below lists other examples synthesized following the general scheme above.

| Starting | | | |
|---|---|---|---|
| from | X | R² | Product |
| 260 | CO | Me | 316 |
| 260 | CO | neopentyl | 196 |
| 429 | CO | Me | 379 |
| 41 | CO | Me | 232 |
| 101 | CO | Me | 243 |
| 8 | CO | Me | 149 |
| 271 | CO₂ | Et | 127 |
| 271 | CO₂ | Me | 14 |
| 167 | CO₂ | Et | 141 |
| 69 | CO₂ | Me | 30 |
| 160 | CO₂ | Me | 221 |
| 160 | CO₂ | Et | 382 |
| 69 | CO₂ | Et | 225 |
| 282 | CO₂ | Me | 249 |
| 282 | CO₂ | Et | 472 |
| 41 | CO₂ | Me | 471 |
| 101 | CO₂ | Me | 239 |
| 101 | CO₂ | Et | 269 |
| 8 | CO₂ | Me | 129 |
| 8 | CO₂ | Et | 298 |
| 160 | SO₂ | Me | 340 |

### Example 5

### General Scheme

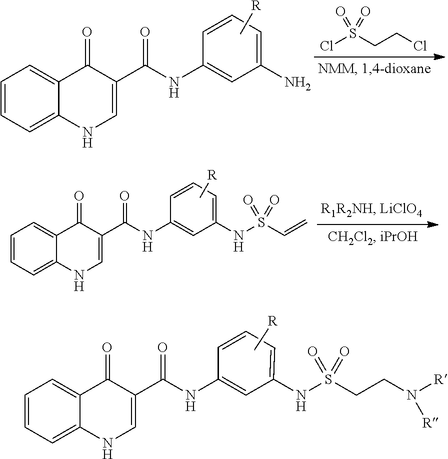

### Specific Example

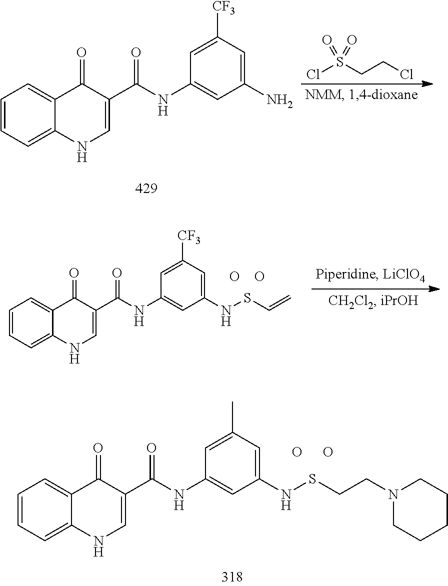

### 4-Oxo-N-[3-(trifluoromethyl)-5-(vinylsulfonamido)phenyl]-1,4-dihydroquinoline-3-carboxamide

To a suspension of N-[3-amino-5-(trifluoromethyl)phenyl]-4-oxo-1H-quinoline-3-carboxamide (429) (500 mg 1.4 mmol) in 1,4-dioxane (4 mL) was added NMM (0.4 mL, 3.6 mmol). β-Chloroethylsulfonyl chloride (0.16 mL, 1.51 mmol) was added under an argon atmosphere. The mixture was stirred at room temperature for 6½ h, after which TLC(CH₂Cl₂-EtOAc, 8:2) showed a new spot with a very similar R_{f} to the starting material. Another 0.5 eq. of NMM was added, and the mixture was stirred at room temperature overnight. LCMS analysis of the crude mixture showed >85% conversion to the desired product. The mixture was concentrated, treated with 1M HCl (5 mL), and extracted with EtOAc (3×10 mL) and CH₂Cl₂ (3×10 mL). The combined organic extracts were dried over Na₂SO₄, filtered, and concentrated to yield 4-oxo-N-[3-(trifluoromethyl)-5-(vinylsulfonamido)phenyl]-1,4-dihydroquinoline-3-carboxamide as an orange foam (0.495 g, 79%), which was used in the next step without further purification. ¹H-NMR (d₆-Acetone, 300 MHz) δ 8.92 (s, 1H), 8.41-8.38 (m, 1H), 7.94 (m, 2H), 7.78 (br s, 2H), 7.53-7.47 (m, 1H), 7.30 (s, 1H), 6.87-6.79 (dd, J=9.9 Hz, 1H), 6.28 (d, J=16.5 Hz, 1H), 6.09 (d, J=9.9 Hz, 1H); ESI-MS 436.4 m/z (MH⁻)

### 318; 4-Oxo-N-[3-[2-(1-piperidyl)ethylsulfonylamino]-5-(trifluoromethyl)phenyl]-1H-quinoline-3-carboxamide

A mixture of 4-oxo-N-[3-(trifluoromethyl)-5-(vinylsulfonamido)phenyl]-1,4-dihydroquinoline-3-carboxamide (50 mg, 0.11 mmol), piperidine (18 μL, 1.6 eq) and LiClO₄ (20 mg, 1.7 eq) was suspended in a 1:1 solution of CH₂Cl₂: isopropanol (1.5 mL). The mixture was refluxed at 75° C. for 18 h. After this time, LCMS analysis showed >95% conversion to the desired product. The crude mixture was purified by reverse-phase HPLC to provide 4-oxo-N-[3-[2-(1-piperidyl)ethylsulfonylamino]-5-(trifluoromethyl)phenyl]-1H-quinoline-3-carboxamide (318) as a yellowish solid (15 mg, 25%). ¹H-NMR (d₆-Acetone, 300 MHz) δ 8.92 (br s, 1H), 8.4 (d, J=8.1 Hz, 1H), 8.05 (br s, 1H), 7.94 (br s, 1H), 7.78 (br s, 2H), 7.53-751 (m, 1H), 7.36 (br s, 1H), 3.97 (t, J=7.2 Hz, 2H), 3.66 (t, J=8 Hz, 2H), 3.31-3.24 (m, 6H), 1.36-1.31 (m, 4H); ESI-MS 489.1 m/z (MH⁺).

The table below lists other examples synthesized following the general scheme above.

| Starting | | |
|---|---|---|
| Intermediate | Amine | Product |
| 429 | morpholine | 272 |
| 429 | dimethylamine | 359 |
| 131 | piperidine | 133 |
| 131 | morpholine | 46 |

### Example 6

### General Scheme

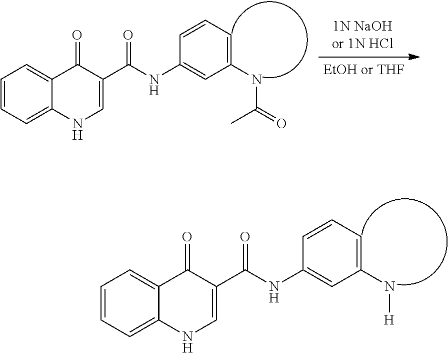 [SMILES: CC(=O)Nc1cccc(NC(=O)c2c[nH]c3ccccc3c2=O)c1 ; Nc1cccc(NC(=O)c2c[nH]c3ccccc3c2=O)c1]

### Specific Example

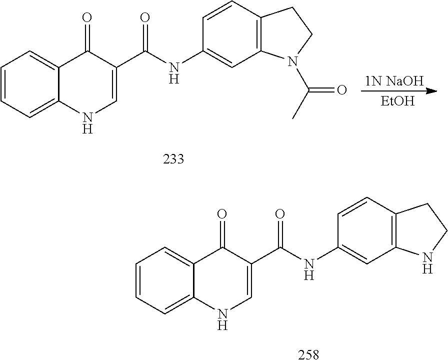

### 258; N-Indolin-6-yl-4-oxo-1H-quinoline-3-carboxamide

A mixture of N-(1-acetylindolin-6-yl)-4-oxo-1H-quinoline-3-carboxamide (233) (43 mg, 0.12 mmol), 1N NaOH solution (0.5 mL) and ethanol (0.5 mL) was heated to reflux for 48 h. The solution was concentrated and the residue was dissolved in DMSO (1 mL) and purified by HPLC (10-99% CH₃CN—H₂O) to yield the product, N-indolin-6-yl-4-oxo-1H-quinoline-3-carboxamide (258) (10 mg, 20%). HPLC ret. time 2.05 min, 10-99% CH₃CN, 5 min run; ESI-MS 306.3 m/z (MH⁺).

The table below lists other examples synthesized following the general scheme above.

| Starting from | Product | Conditions | Solvent |
|---|---|---|---|
| DC-8-I | 386 | NaOH | EtOH |
| DC-9-I | 10 | HCl | EtOH |
| 175 | 22 | HCl | EtOH |
| 109 | 35 | HCl | EtOH |
| 334 | 238 | NaOH | EtOH |
| DC-10-I | 105 | NaOH | THF |

### Example 2

### General Scheme

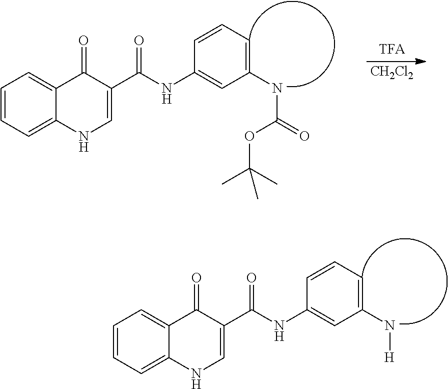 [SMILES: CC(C)(C)OC(=O)Nc1cccc(NC(=O)c2c[nH]c3ccccc3c2=O)c1 ; "CH2Cl2" ; Nc1cccc(NC(=O)c2c[nH]c3ccccc3c2=O)c1]

### Specific Example

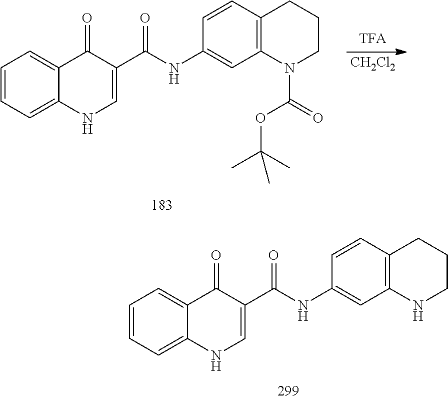

### 299; 4-Oxo-N-(1,2,3,4-tetrahydroquinolin-7-yl)-1H-quinoline-3-carboxamide

A mixture of 7-[(4-oxo-1H-quinolin-3-yl)carbonylamino]-1,2,3,4-tetrahydroquinoline-1-carboxylic acid tert-butyl ester (183) (23 mg, 0.05 mmol), TFA (1 mL) and CH₂Cl₂ (1 mL) was stirred at room temperature overnight. The solution was concentrated and the residue was dissolved in DMSO (1 mL) and purified by HPLC (10-99% CH₃CN—H₂O) to yield the product, 4-oxo-N-(1,2,3,4-tetrahydroquinolin-7-yl)-1H-quinoline-3-carboxamide (299) (7 mg, 32%). HPLC ret. time 2.18 min, 10-99% CH₃CN, 5 min run; ESI-MS 320.3 m/z (MH⁺).

### Another Example

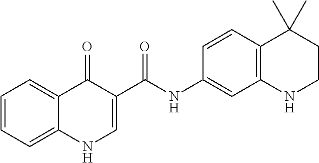 [SMILES: CC1(C)CCNc2cc(NC(=O)c3c[nH]c4ccccc4c3=O)ccc21]

### 300; N-(4,4-Dimethyl-1,2,3,4-tetrahydroquinolin-7-yl)-4-oxo-1H-quinoline-3-carboxamide

N-(4,4-Dimethyl-1,2,3,4-tetrahydroquinolin-7-yl)-4-oxo-1H-quinoline-3-carboxamide (300) was synthesized following the general scheme above starting from 4,4-dimethyl-7-[(4-oxo-1H-quinolin-3-yl)carbonylamino]-1,2,3,4-tetrahydroquinoline-1-carboxylic acid tert-butyl ester (108). Yield (33%). ¹H NMR (400 MHz, DMSO-d₆) δ 13.23 (d, J=6.6 Hz, 1H), 12.59 (s, 1H), 8.87 (d, J=6.8 Hz, 1H), 8.33 (d, J=7.7 Hz, 1H), 7.86-7.79 (m, 3H), 7.58-7.42 (m, 3H), 3.38 (m, 2H), 1.88 (m, 2H), 1.30 (s, 6H); HPLC ret. time 2.40 min, 10-99% CH₃CN, 5 min run; ESI-MS 348.2 m/z (MH⁺).

### Other

### Example 1

### General Scheme

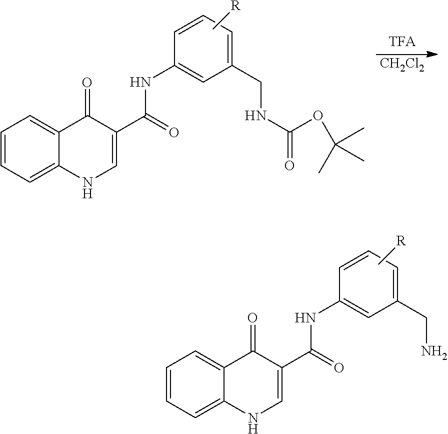

### Specific Example

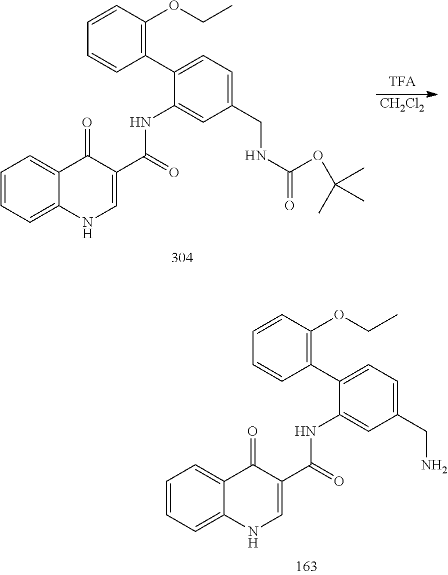 [SMILES: CCOc1ccccc1-c1ccc(CNC(=O)OC(C)(C)C)cc1NC(=O)c1c[nH]c2ccccc2c1=O ; "CH2Cl2" ; CCOc1ccccc1-c1ccc(CN)cc1NC(=O)c1c[nH]c2ccccc2c1=O]

### 163; 4-Oxo-1,4-dihydro-quinoline-3-carboxylic acid (4-aminomethyl-2′-ethoxy-biphenyl-2-yl)-amide

{2′-Ethoxy-2-[(4-oxo-1,4-dihydroquinoline-3-carbonyl)-amino]-biphenyl-4-ylmethyl}-carbamic acid tert-butyl ester (304) (40 mg, 0.078 mmol) was stirred in a CH₂Cl₂/TFA mixture (3:1, 20 mL) at room temperature for 1 h. The volatiles were removed on a rotary evaporator. The crude product was purified by preparative HPLC to afford 4-oxo-1,4-dihydroquinoline-3-carboxylix acid (4-aminomethyl-2′-ethoxybiphenyl-2-yl)amine (163) as a tan solid (14 mg. 43%). ¹H NMR (300 MHz, DMSO-d₆) δ 12.87 (d, J=6.3 Hz, 1H), 11.83 (s, 1H), 8.76 (d, J=6.3 Hz, 1H), 8.40 (s, 1H), 8.26 (br s, 2H), 8.01 (dd, J=8.4 Hz, J=1.5 Hz, 1H), 7.75 (dt, J=8.1 Hz, J=1.2 Hz, 1H), 7.67 (d, J=7.8 Hz, 1H), 7.47-7.37 (m, 2H), 7.24 (s, 2H), 7.15 (dd, J=7.5 Hz, J=1.8 Hz, 1H), 7.10 (d, J=8.1 Hz, 1H), 7.02 (dt, J=7.5 Hz, J=0.9 Hz, 1H), 4.09 (m, 2H), 4.04 (q, J=6.9 Hz, 2H), 1.09 (t, J=6.9 Hz, 3H); HPLC ret. time 1.71 min, 10-100% CH₃CN, 5 min gradient; ESI-MS 414.1 m/z (MH⁺).

### Another Example

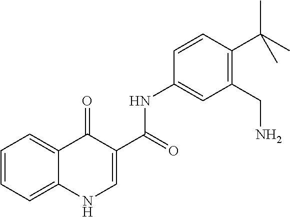 [SMILES: CC(C)(C)c1ccc(NC(=O)c2c[nH]c3ccccc3c2=O)cc1CN]

### 390; N-[3-(Aminomethyl)-4-tert-butyl-phenyl]-4-oxo-1H-quinoline-3-carboxamide

N-[3-(Aminomethyl)-4-tert-butyl-phenyl]-4-oxo-1H-quinoline-3-carboxamide (390) was synthesized following the general scheme above starting from [5-[(4-oxo-1H-quinolin-3-yl)carbonylamino]-2-tert-butyl-phenyl]methylaminoformic acid tert-butyl ester (465). HPLC ret. time 2.44 min, 10-99% CH₃CN, 5 min gradient; ESI-MS m/z 350.3 (M+H)⁺.

### Example 2

### General Scheme

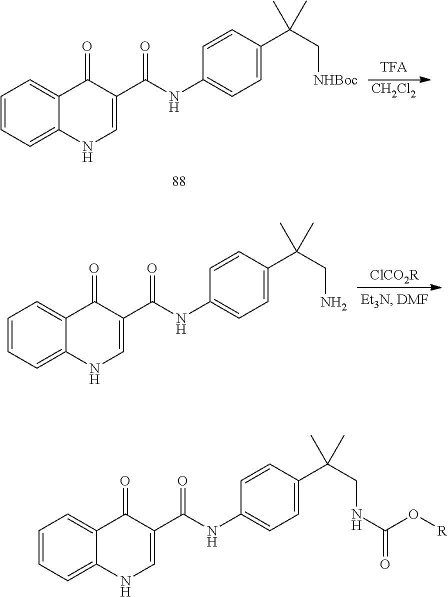

### Specific Example

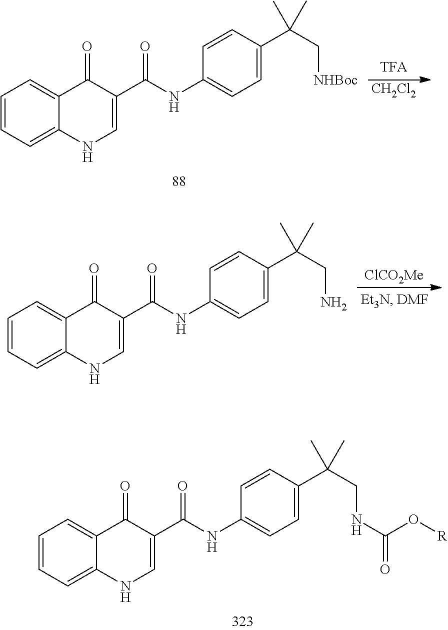

### 3-(2-(4-(1-Amino-2-methylpropan-2-yl)phenyl)acetyl)quinolin-4(1H)-one

(2-Methyl-2-{4-[2-oxo-2-(4-oxo-1,4-dihydro-quinolin-3-yl)-ethyl]-phenyl}-propyl)-carbamic acid tert-butyl ester (88) (0.50 g, 1.15 mmol), TFA (5 mL) and CH₂Cl₂ (5 mL) were combined and stirred at room temperature overnight. The reaction mixture was then neutralized with 1N NaOH. The precipitate was collected via filtration to yield the product 3-(2-(4-(1-amino-2-methylpropan-2-yl)phenyl)acetyl)quinolin-4(1H)-one as a brown solid (651 mg, 91%). HPLC ret. time 2.26 min, 10-99% CH₃CN, 5 min run; ESI-MS 336.5 m/z (MH⁺).

### 323; [2-Methyl-2-[4-[(4-oxo-1H-quinolin-3-yl)carbonylamino]phenyl]-propyl]aminoformic acid methyl ester

Methyl chloroformate (0.012 g, 0.150 mmol) was added to a solution of 3-(2-(4-(1-amino-2-methylpropan-2-yl)phenyl)acetyl)quinolin-4(1H)-one (0.025 g, 0.075 mmol), TEA (0.150 mmol, 0.021 mL) and DMF (1 mL) and stirred at room temperature for 1 h. Then piperidine (0.074 ml, 0.750 mmol) was added and the reaction was stirred for another 30 min. The reaction mixture was filtered and purified by preparative HPLC (10-99% CH₃CN—H₂O) to yield the product [2-methyl-2-[4-[(4-oxo-1H-quinolin-3-yl)carbonylamino]phenyl]-propyl]aminoformic acid methyl ester (323). ¹H NMR (400 MHz, DMSO-d6) δ 12.94 (br s, 1H), 12.44 (s, 1H), 8.89 (s, 1H), 8.33 (dd, J=8.2, 1.1 Hz, 1H), 7.82 (t, J=8.3 Hz, 1H), 7.76 (d, J=7.7 Hz, 1H), 7.67 (d, J=8.8 Hz, 2H), 7.54 (t, J=8.1 Hz, 1H), 7.35 (d, J=8.7 Hz, 2H), 7.02 (t, J=6.3 Hz, 1H), 3.50 (s, 3H), 3.17 (d, J=6.2 Hz, 2H), 1.23 (s, 6H); HPLC ret. time 2.93 min, 10-99% CH₃CN, 5 min run; ESI-MS 394.0 m/z (MH⁺).

The table below lists other examples synthesized following the general scheme above.

| Product | Chloroformate |
|---|---|
| 119 | Ethyl chloroformate |
| 416 | Propyl chloroformate |
| 460 | Butyl chloroformate |
| 251 | Isobutyl chloroformate |
| 341 | Neopentyl chloroformate |
| 28 | 2-methoxyethyl chloroformate |
| 396 | (tetrahydrofuran-3-yl)methyl chloroformate |

### Example 3

### General Scheme

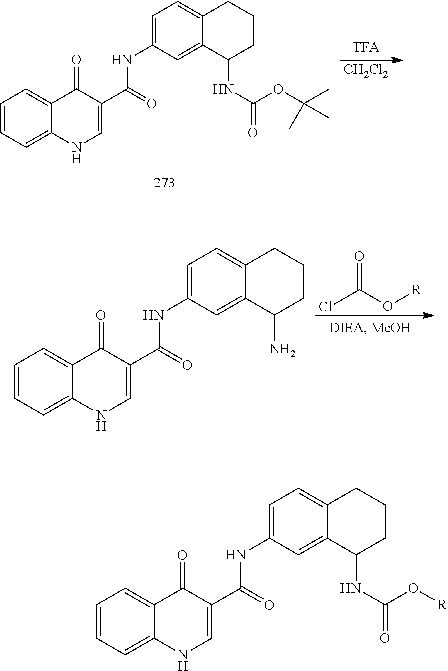

### Specific Example

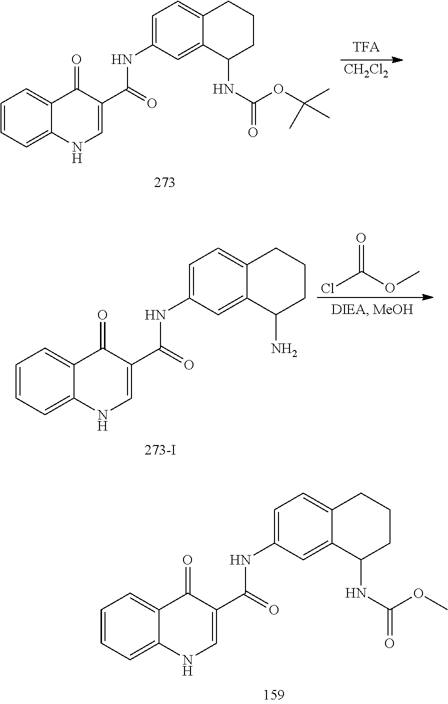

### 273-I; N-(1-Aminotetralin-7-yl)-4-oxo-1H-quinoline-3-carboxamide

To a solution of [7-[(4-oxo-1H-quinolin-3-yl)carbonylamino]tetralin-1-yl]aminoformic acid tert-butyl ester (273) (250 mg, 0.6 mmol) in dichloromethane (2 mL) was added TFA (2 mL). The reaction was stirred at room temperature for 30 min. More dichloromethane (10 mL) was added to the reaction mixture and the solution was washed with sat. NaHCO₃ solution (5 mL). A precipitate began to form in the organic layer so the combined organic layers were concentrated to yield N-(1-aminotetralin-7-yl)-4-oxo-1H-quinoline-3-carboxamide (273-I) (185 mg, 93%). HPLC ret. time 1.94 min, 10-99% CH₃CN, 5 min run; ESI-MS 334.5 m/z (MH⁺).

### 159; [7-[(4-Oxo-1H-quinolin-3-yl)carbonylamino]tetralin-1-yl]aminoformic acid methyl ester

To a solution of N-(1-aminotetralin-7-yl)-4-oxo-1H-quinoline-3-carboxamide (273-I) (65 mg, 0.20 mmol) and DIEA (52 μL, 0.29 mmol) in methanol (1 mL) was added methyl chloroformate (22 μL, 0.29 mmol). The reaction was stirred at room temperature for 1 h. LCMS analysis of the reaction mixture showed peaks corresponding to both the single and bis addition products. Piperidine (2 mL) was added and the reaction was stirred overnight after which only the single addition product was observed. The resulting solution was filtered and purified by HPLC (10-99% CH₃CN—H₂O) to yield the product, [7-[(4-oxo-1H-quinolin-3-yl)carbonylamino]tetralin-1-yl]aminoformic acid methyl ester (159) (27 mg, 35%). HPLC ret. time 2.68 min, 10-99% CH₃CN, 5 min run; ESI-MS 392.3 m/z (MH⁺).

### Another Example

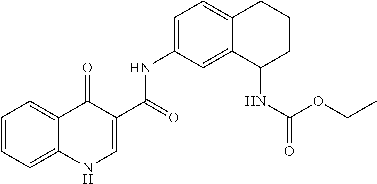 [SMILES: CCOC(=O)NC1CCCc2ccc(NC(=O)c3c[nH]c4ccccc4c3=O)cc21]

### 482; [7-[(4-Oxo-1H-quinolin-3-yl)carbonylamino]tetralin-1-yl]aminoformic acid ethyl ester

[7-[(4-Oxo-1H-quinolin-3-yl)carbonylamino]tetralin-1-yl]aminoformic acid ethyl ester (482) was synthesized following the general scheme above, from amine (273-I) and ethyl chloroformate. Overall yield (18%). HPLC ret. time 2.84 min, 10-99% CH₃CN, 5 min run; ESI-MS 406.5 m/z (MH⁺).

Set forth below is the characterizing data for compounds of the present invention prepared according to the above Examples.

TABLE II.A-3

| Cmd | LC-MS | LC-RT |
|---|---|---|
| No. | M + 1 | min |
| 1 | 444.3 | 3.19 |
| 2 | 350.1 | 3.8 |
| 3 | 455.3 | 3.75 |
| 4 | 350.3 | 2.81 |
| 5 | 337.3 | 2.76 |
| 6 | 351.4 | 3 |
| 7 | 472.3 | 3.6 |
| 8 | 307.1 | 1.21 |
| 9 | 344.1 | 2.43 |
| 10 | 334.2 | 2.2 |
| 11 | 408.1 | 2.91 |
| 12 | 383.1 | 2.63 |
| 13 | 346.3 | 3.48 |
| 14 | 394.3 | 3.07 |
| 15 | 296.3 | 2.68 |
| 16 | 307.3 | 3.38 |
| 17 | 338.3 | 3.74 |
| 18 | 352.9 | 3.62 |
| 19 | 316.3 | 2.71 |
| 20 | 371.3 | 3.53 |
| 21 | 421.1 | 2.66 |
| 22 | 332.2 | 2.21 |
| 23 | 457.5 | 3.56 |
| 24 | 398.3 | 3.13 |
| 25 | 397.1 | 2.38 |
| 26 | 348.1 | 2.51 |
| 27 | 446.2 | 2.33 |
| 28 | 438.4 | 2.9 |
| 29 | 307.1 | 3.32 |
| 30 | 379.1 | 2.62 |
| 31 | 278.9 | 3.03 |
| 32 | 338.2 | 3 |
| 33 | 303.9 | 2.83 |
| 34 | 397.1 | 4.19 |
| 35 | 362.2 | 2.53 |
| 36 | 307.3 | 3.25 |
| 37 | 303.9 | 2.98 |
| 38 | 380.3 | 3.33 |
| 39 | 480.5 | 3.82 |
| 40 | 309.1 | 2.46 |
| 41 | 321.1 | 1.88 |
| 42 | 460.0 | 3.71 |
| 43 | 457.5 | 3.6 |
| 44 | 336.1 | 2.95 |
| 45 | 308.1 | 3.18 |
| 46 | 490.1 | 1.89 |
| 47 | 375.3 | 3.33 |
| 48 | 317.1 | 3.06 |
| 49 | 400.1 | 2.88 |
| 50 | 307.3 | 3.08 |
| 51 | 521.5 | 3.79 |
| 52 | 354.1 | 3.02 |
| 53 | 266.1 | 1.99 |
| 54 | 323.3 | 2.97 |
| 55 | 366.3 | 2.6 |
| 56 | 335.4 | 3.18 |
| 57 | 403.1 | 2.86 |
| 58 | 364.3 | 3.02 |
| 59 | 412.1 | 3.31 |
| 60 | 422.2 | 3.53 |
| 61 | 293.1 | 3.05 |
| 62 | 349.1 | 3.4 |
| 63 | 376.1 | 2.89 |
| 64 | 321.1 | 2.31 |
| 65 | 381.5 | 1.85 |
| 66 | 345.1 | 3.32 |
| 67 | 332.3 | 3.17 |
| 68 | 398.1 | 2.85 |
| 69 | 322.5 | 2.37 |
| 70 | 341.1 | 2.15 |
| 71 | 426.1 | 2.6 |
| 72 | 293.1 | 3.27 |
| 73 | 380.9 | 2.4 |
| 74 | 334.1 | 3.32 |
| 75 | 316.3 | 2.43 |
| 76 | 376.1 | 2.97 |
| 77 | 322.5 | 2.93 |
| 78 | 344.1 | 2.38 |
| 79 | 372.1 | 3.07 |
| 80 | 295.3 | 2.78 |
| 81 | 336.3 | 2.73 |
| 82 | 350.3 | 2.11 |
| 83 | 365.1 | 2.76 |
| 84 | 280.3 | 2.11 |
| 85 | 408.0 | 3.25 |
| 86 | 370.3 | 2.08 |
| 87 | 357.1 | 3.5 |
| 88 | 436.3 | 3.37 |
| 89 | 303.9 | 3.1 |
| 90 | 321.1 | 3.43 |
| 91 | 355.2 | 3.47 |
| 92 | 295.2 | 3.84 |
| 93 | 371.0 | 2.75 |
| 94 | 294.2 | 2.06 |
| 95 | 290.1 | 2.78 |
| 96 | 343.0 | 2.75 |
| 97 | 402.1 | 2.59 |
| 98 | 349.1 | 1.96 |
| 99 | 334.1 | 3.13 |
| 100 | 303.9 | 2.63 |
| 101 | 322.5 | 2.35 |
| 102 | 443.1 | 3.97 |
| 103 | 411.2 | 3.85 |
| 104 | 318.0 | 2.94 |
| 105 | 322.2 | 2.4 |
| 106 | 350.3 | 2.86 |
| 107 | 420.2 | 3.37 |
| 108 | 448.2 | 3.77 |
| 109 | 404.5 | 3.17 |
| 110 | 303.9 | 2.75 |
| 111 | 333.1 | 3 |
| 112 | 348.5 | 3.07 |
| 113 | 318.3 | 3.02 |
| 114 | 499.2 | 3.74 |
| 115 | 330.1 | 2.67 |
| 116 | 320.2 | 3.18 |
| 117 | 349.1 | 1.32 |
| 118 | 379.1 | 2.61 |
| 119 | 408.4 | 3.07 |
| 120 | 309.1 | 2.93 |
| 121 | 333.1 | 3.69 |
| 122 | 325.1 | 2.66 |
| 123 | 330.1 | 2.64 |
| 124 | 378.3 | 3.4 |
| 125 | 294.3 | 2.21 |
| 126 | 411.1 | 3.06 |
| 127 | 408.5 | 3.22 |
| 128 | 369.1 | 3.53 |
| 129 | 365.1 | 1.74 |
| 130 | 440.2 | 3.57 |
| 131 | 313.0 | 2.4 |
| 132 | 365.9 | 2.73 |
| 133 | 488.1 | 1.97 |
| 134 | 402.1 | 2.25 |
| 135 | 384.1 | 2.94 |
| 136 | 393.1 | 4.33 |
| 137 | 580.5 | 4.1 |
| 138 | 376.1 | 2.98 |
| 139 | 408.0 | 3.17 |
| 140 | 346.1 | 4 |
| 141 | 366.3 | 2.89 |
| 142 | 321.3 | 3.58 |
| 143 | 355.2 | 3.45 |
| 144 | 281.3 | 2.49 |
| 145 | 376.2 | 2.98 |
| 146 | 306.3 | 2.51 |
| 147 | 376.3 | 3.27 |
| 148 | 415.5 | 2.79 |
| 149 | 349.1 | 1.45 |
| 150 | 430.0 | 3.29 |
| 151 | 360.0 | 3 |
| 152 | 322.3 | 2.31 |
| 153 | 425.1 | 4.52 |
| 154 | 401.3 | 3.77 |
| 155 | 266.1 | 2.11 |
| 156 | 424.1 | 3.12 |
| 157 | 321.0 | 2.13 |
| 158 | 380.2 | 3.05 |
| 159 | 392.3 | 2.68 |
| 160 | 321.1 | 1.34 |
| 161 | 409.2 | 3.82 |
| 162 | 296.3 | 2.61 |
| 163 | 413.1 | 1.71 |
| 164 | 333.1 | 3.33 |
| 165 | 344.1 | 2.41 |
| 166 | 398.1 | 2.83 |
| 167 | 294.3 | 2.12 |
| 168 | 265.9 | 1.96 |
| 169 | 318 | 2.98 |
| 170 | 300.3 | 3.08 |
| 171 | 408.0 | 3.08 |
| 172 | 396.0 | 3.14 |
| 173 | 280.3 | 2.14 |
| 174 | 388.0 | 2.58 |
| 175 | 374.2 | 2.85 |
| 176 | 349.1 | 3.38 |
| 177 | 337.1 | 3.5 |
| 178 | 413.3 | 4 |
| 179 | 308.5 | 2.33 |
| 180 | 307.3 | 3.08 |
| 181 | 354.1 | 2.97 |
| 182 | 358.1 | 2.89 |
| 183 | 420.3 | 3.47 |
| 184 | 372.3 | 2.66 |
| 185 | 414.1 | 2.96 |
| 186 | 372.3 | 3.59 |
| 187 | 346.3 | 2.9 |
| 188 | 376.2 | 2.95 |
| 189 | 370.9 | 3.38 |
| 190 | 392.0 | 3.09 |
| 191 | 316.3 | 2.1 |
| 192 | 280.3 | 2.13 |
| 193 | 326.3 | 3.02 |
| 194 | 290.1 | 2.98 |
| 195 | 280.3 | 2.14 |
| 196 | 434.5 | 3.38 |
| 197 | 334.1 | 3.15 |
| 198 | 283.1 | 3 |
| 199 | 354.1 | 2.96 |
| 200 | 335.5 | 2.49 |
| 201 | 303.9 | 3.08 |
| 202 | 404.0 | 3.19 |
| 203 | 394.3 | 3.42 |
| 204 | 349.3 | 3.32 |
| 205 | 455.5 | 3.74 |
| 206 | 386.1 | 3.5 |
| 207 | 390.3 | 2.71 |
| 208 | 429.7 | 3.89 |
| 209 | 294.1 | 2.39 |
| 210 | 385.2 | 3.72 |
| 211 | 351.3 | 3.53 |
| 212 | 360.9 | 2.45 |
| 213 | 408.0 | 3.3 |
| 214 | 358.1 | 2.7 |
| 215 | 265.3 | 3.07 |
| 216 | 305.3 | 2.27 |
| 217 | 305.3 | 2.41 |
| 218 | 413.2 | 3.98 |
| 219 | 266.9 | 2.48 |
| 220 | 409.0 | 3.35 |
| 221 | 379.1 | 2.68 |
| 222 | 324.3 | 3.27 |
| 223 | 386.1 | 3.14 |
| 224 | 466.3 | 3.08 |
| 225 | 393.1 | 2.75 |
| 226 | 306.1 | 3.6 |
| 227 | 381.1 | 2.24 |
| 228 | 371.1 | 2.84 |
| 229 | 311.1 | 2.93 |
| 230 | 318.1 | 2.81 |
| 231 | 471.3 | 3.41 |
| 232 | 363.1 | 2.57 |
| 233 | 348.5 | 2.75 |
| 234 | 372.3 | 3.2 |
| 235 | 308.4 | 2.12 |
| 236 | 333.1 | 3.35 |
| 237 | 410.3 | 2.96 |
| 238 | 489.4 | 2.78 |
| 239 | 379.0 | 2.62 |
| 240 | 370.9 | 3.65 |
| 241 | 316.3 | 2.61 |
| 242 | 348.3 | 3.08 |
| 243 | 363.0 | 2.44 |
| 244 | 358.1 | 3.48 |
| 245 | 425.1 | 3.69 |
| 246 | 292.9 | 3.2 |
| 247 | 432.1 | 3.23 |
| 248 | 336.3 | 2.46 |
| 249 | 365.0 | 2.54 |
| 250 | 352.3 | 2.53 |
| 251 | 436.2 | 3.38 |
| 252 | 368.9 | 3.17 |
| 253 | 424.1 | 3.25 |
| 254 | 340.1 | 3.08 |
| 255 | 526.5 | 3.89 |
| 256 | 306.1 | 2.4 |
| 257 | 297.3 | 3.28 |
| 258 | 306.3 | 2.05 |
| 259 | 360.3 | 3.46 |
| 260 | 336.3 | 2.33 |
| 261 | 368.1 | 3.08 |
| 262 | 352.3 | 2.7 |
| 263 | 372.9 | 3.69 |
| 264 | 353.1 | 3.42 |
| 265 | 354.9 | 3.4 |
| 266 | 405.3 | 4.05 |
| 267 | 357.1 | 3.43 |
| 268 | 400.3 | 6.01 |
| 269 | 393.0 | 2.75 |
| 270 | 329.3 | 3.02 |
| 271 | 336.5 | 2.75 |
| 272 | 524.1 | 1.87 |
| 273 | 434.5 | 3.17 |
| 274 | 493.5 | 3.46 |
| 275 | 427.1 | 3.93 |
| 276 | 414.3 | 2.81 |
| 277 | 358.1 | 2.89 |
| 278 | 408.1 | 3.09 |
| 279 | 386.1 | 2.88 |
| 280 | 316.3 | 2.06 |
| 281 | 293.1 | 3.22 |
| 282 | 307.1 | 1.22 |
| 283 | 370.1 | 3 |
| 284 | 305.3 | 2.57 |
| 285 | 376.1 | 2.88 |
| 286 | 319.1 | 3.35 |
| 287 | 411.2 | 4.15 |
| 288 | 413.3 | 3.8 |
| 289 | 297.3 | 3.25 |
| 290 | 382.1 | 3.19 |
| 291 | 371.0 | 3.57 |
| 292 | 391.1 | 3.69 |
| 293 | 330.3 | 3.05 |
| 294 | 303.9 | 2.67 |
| 295 | 334.3 | 2.26 |
| 296 | 365.3 | 3.6 |
| 297 | 358.3 | 3.26 |
| 298 | 379.1 | 1.91 |
| 299 | 320.3 | 2.18 |
| 300 | 348.2 | 2.4 |
| 301 | 346.3 | 2.26 |
| 302 | 370.1 | 2.28 |
| 303 | 362.2 | 2.51 |
| 304 | 513.2 | 3.66 |
| 305 | 370.1 | 2.98 |
| 306 | 384.1 | 3.11 |
| 307 | 374.0 | 3.05 |
| 308 | 304.1 | 2.71 |
| 309 | 316.3 | 2.83 |
| 310 | 320.1 | 3.73 |
| 311 | 344.9 | 3.43 |
| 312 | 400.1 | 2.86 |
| 313 | 358.1 | 2.8 |
| 314 | 335.1 | 3.52 |
| 315 | 293.1 | 2.9 |
| 316 | 378.5 | 2.84 |
| 317 | 333.2 | 2.91 |
| 318 | 522.1 | 1.8 |
| 319 | 373.3 | 3.59 |
| 320 | 360.1 | 3.5 |
| 321 | 453.5 | 3.12 |
| 322 | 349.3 | 3.7 |
| 323 | 394.0 | 2.93 |
| 324 | 320.1 | 3.81 |
| 325 | 321.3 | 3.22 |
| 326 | 418.0 | 2.5 |
| 327 | 424.2 | 3.2 |
| 328 | 307.1 | 2.76 |
| 329 | 396.3 | 3.72 |
| 330 | 299.3 | 3.02 |
| 331 | 308.3 | 2.25 |
| 332 | 288.0 | 2.5 |
| 333 | 379.1 | 2.61 |
| 334 | 531.3 | 3.26 |
| 335 | 322.3 | 2.41 |
| 336 | 321.5 | 3.52 |
| 337 | 407.5 | 3.37 |
| 338 | 318.3 | 2.73 |
| 339 | 329.0 | 2.75 |
| 340 | 399.1 | 2.6 |
| 341 | 450.4 | 3.56 |
| 342 | 422.3 | 3.41 |
| 343 | 403.3 | 2.73 |
| 344 | 384.1 | 3.07 |
| 345 | 322.2 | 2.96 |
| 346 | 333.1 | 3.38 |
| 347 | 494.5 | 1.97 |
| 348 | 384.1 | 3.12 |
| 349 | 405.3 | 2.85 |
| 350 | 315.1 | 3.23 |
| 351 | 332.3 | 3.18 |
| 352 | 447.5 | 3.17 |
| 353 | 436.3 | 3.53 |
| 354 | 390.3 | 2.36 |
| 355 | 370.9 | 3.37 |
| 356 | 335.0 | 1.81 |
| 357 | 346.3 | 3.08 |
| 358 | 338.2 | 3.15 |
| 359 | 482.1 | 1.74 |
| 360 | 331.3 | 3.07 |
| 361 | 400.1 | 2.91 |
| 362 | 355.5 | 3.46 |
| 363 | 388.1 | 2.92 |
| 364 | 330.3 | 2.68 |
| 365 | 307.1 | 2.6 |
| 366 | 408.1 | 3.09 |
| 367 | 408.0 | 3.14 |
| 368 | 338.2 | 2.33 |
| 369 | 358.1 | 3.29 |
| 370 | 299.1 | 3.03 |
| 371 | 365.0 | 3.27 |
| 372 | 362.1 | 2.66 |
| 373 | 305.3 | 3.38 |
| 374 | 350.3 | 3.01 |
| 375 | 319.3 | 3.4 |
| 376 | 382.3 | 3.48 |
| 377 | 340.2 | 3.08 |
| 378 | 310.3 | 2.07 |
| 379 | 389.0 | 2.53 |
| 380 | 309.3 | 3.02 |
| 381 | 360.2 | 3.18 |
| 382 | 393.1 | 2.84 |
| 383 | 332.3 | 3.2 |
| 384 | 376.1 | 2.87 |
| 385 | 393.9 | 3.32 |
| 386 | 334.3 | 2.3 |
| 387 | 347.1 | 3.22 |
| 388 | 424.1 | 3.3 |
| 389 | 355.3 | 3.65 |
| 390 | 350.3 | 2.44 |
| 391 | 396.1 | 3.43 |
| 392 | 300.3 | 2.86 |
| 393 | 399.4 | 2.12 |
| 394 | 293.1 | 3.17 |
| 395 | 433.5 | 4.21 |
| 396 | 464.4 | 2.97 |
| 397 | 341.3 | 3.45 |
| 398 | 434.3 | 3.1 |
| 399 | 335.0 | 1.75 |
| 400 | 351.3 | 2.11 |
| 401 | 368.1 | 3.09 |
| 402 | 342.1 | 2.96 |
| 403 | 423.1 | 4.45 |
| 404 | 440.3 | 2.87 |
| 405 | 299.3 | 3.16 |
| 406 | 547.3 | 3.74 |
| 407 | 371.3 | 3.8 |
| 408 | 295.3 | 2.9 |
| 409 | 335.1 | 1.82 |
| 410 | 432.1 | 3.41 |
| 411 | 299.1 | 3.17 |
| 412 | 376.2 | 2.93 |
| 413 | 357.1 | 3.37 |
| 414 | 305.3 | 2.11 |
| 415 | 351.5 | 3.44 |
| 416 | 422.4 | 3.23 |
| 417 | 396.0 | 2.67 |
| 418 | 308.3 | 2.23 |
| 419 | 322.3 | 2.48 |
| 420 | 379.1 | 3.2 |
| 421 | 419.2 | 3.82 |
| 422 | 333.1 | 2.48 |
| 423 | 376.3 | 3.02 |
| 424 | 374.0 | 3.06 |
| 425 | 306.1 | 3.53 |
| 426 | 371.3 | 2.95 |
| 427 | 420.3 | 3.3 |
| 428 | 337.2 | 3.32 |
| 429 | 348.3 | 2.98 |
| 430 | 321.3 | 3.22 |
| 431 | 280.3 | 2.09 |
| 432 | 382.1 | 3.22 |
| 433 | 393.2 | 3.71 |
| 434 | 293.1 | 3.12 |
| 435 | 376.3 | 3.22 |
| 436 | 400.1 | 2.88 |
| 437 | 309.3 | 2.82 |
| 438 | 427.5 | 3.87 |
| 439 | 295.3 | 2.8 |
| 440 | 395.3 | 3.61 |
| 441 | 425.0 | 2.67 |
| 442 | 412.3 | 3.35 |
| 443 | 317.3 | 2.45 |
| 444 | 379.2 | 3.42 |
| 445 | 305.5 | 3.08 |
| 446 | 353.1 | 2.85 |
| 447 | 290.1 | 2.88 |
| 448 | 321.3 | 3.5 |
| 449 | 279.1 | 3.22 |
| 450 | 308.1 | 1.97 |
| 451 | 318.1 | 3.28 |
| 452 | 290.1 | 3.32 |
| 453 | 314.1 | 2.75 |
| 454 | 355.1 | 3.58 |
| 455 | 398.1 | 3.6 |
| 456 | 365.1 | 3.65 |
| 457 | 350.3 | 2.26 |
| 458 | 381.2 | 3.19 |
| 459 | 279.3 | 2.9 |
| 460 | 436.2 | 3.38 |
| 461 | 341.3 | 3.23 |
| 462 | 349.1 | 1.9 |
| 463 | 292.1 | 3.35 |
| 464 | 409.4 | 4.03 |
| 465 | 450.5 | 3.65 |
| 466 | 349.3 | 3.5 |
| 467 | 307.3 | 2.98 |
| 468 | 279.1 | 2.98 |
| 469 | 409.1 | 3.69 |
| 470 | 373.3 | 3.64 |
| 471 | 379.0 | 2.73 |
| 472 | 379.0 | 2.67 |
| 473 | 363.3 | 3.64 |
| 474 | 336.3 | 2.8 |
| 475 | 334.3 | 3.23 |
| 476 | 362.1 | 3.42 |
| 477 | 283.9 | 2.8 |
| 478 | 360.3 | 3.44 |
| 479 | 334.3 | 2.59 |
| 480 | 323.5 | 3.22 |
| 481 | 315.3 | 3.25 |
| 482 | 406.5 | 2.84 |
| 483 | 409.5 | 4.35 |
| 484 | 349.1 | 2.16 |
| 485 | 363.1 | 2.15 |

NMR data for selected compounds is shown below in Table 2-A:

| Compound | |
|---|---|
| No. | NMR Data |
| 2 | 1H NMR (300 MHz, CDCl₃) δ 12.53 (s, 1H), 11.44 (br d, J = 6.0 Hz, 1H), |
| | 9.04 (d, J = 6.7 Hz, 1H), 8.43 (d, J = 7.8 Hz, 1H), 7.51 (t, J = 7.3 Hz, 1H), |
| | 7.43 (t, J = 7.5 Hz, 1H), 7.33-7.21 (m, 3H), 7.10 (d, J = 8.2 Hz, 1H), 3.79 |
| | (s, 3H), 1.36 (s, 9H) |
| 5 | H NMR (400 MHz, DMSO-d6) δ 12.94 (bs, 1H), 12.41 (s, 1H), 8.88 (s, 1H), |
| | 8.34 (dd, J = 8, 1 Hz, 1H), 7.82 (ddd, J = 8, 8, 1 Hz, 1H), 7.75 (d, J = 8 Hz, 1H), |
| | 7.64 (dd, J = 7, 2 HZ, 2H), 7.54 (ddd, J = 8, 8, 1 Hz, 1H), 7.35 (dd, J = 7, 2 Hz, |
| | 2H), 4.66 (t, J = 5 Hz, 1H), 3.41 (d, J = 5 Hz, 2H), 1.23 (s, 6H). |
| 8 | 1H NMR (CD3OD, 300 MHz) δ 8.86 (s, 1H), 8.42 (d, J = 8.5 Hz, 1H), 7.94 |
| | (s, 1H), 7.81 (t, J = 8.3 Hz, 1H), 7.67 (d, J = 8.3 Hz, 1H), 7.54-7.47 (m, 2H), |
| | 7.38 (d, J = 8.5 Hz, 1H), 2.71 (q, J = 7.7 Hz, 2H), 1.30 (t, J = 7.4 Hz, 3H). |
| 10 | H NMR (400 MHz, DMSO-d6) δ 13.02 (d, J = 6.4 Hz, 1H), 12.58 (s, 1H), |
| | 8.87 (d, J = 6.8 Hz, 1H), 8.33 (dd, J = 8.1, 1.2 Hz, 1H), 7.89-7.77 (m, 3H), |
| | 7.56 (t, J = 8.1 Hz, 1H), 7.39 (d, J = 7.8 Hz, 1H), 7.26 (d, J = 8.4 Hz, 1H), |
| | 3.23 (m, 2H), 2.81 (m, 2H), 1.94 (m, 2H), 1.65 (m, 2H) |
| 13 | H NMR (400 MHz, DMSO-d6) δ 13.05 (bs, 1H), 12.68 (s, 1H), 8.89 (s, 1H), |
| | 8.35 (t, J = 2.5 Hz, 1H), 8.32 (d, J = 1.1 Hz, 1H), 7.85-7.76 (m, 3H), |
| | 7.58-7.54 (m, 2H), 1.47 (s, 9H) |
| 14 | H NMR (400 MHz, DMSO-d6) δ 1.32 (s, 9H), 3.64 (s, 3H), 7.36 (d, J = 8.4 Hz, |
| | 1H), 7.55 (m, 3H), 7.76 (d, J = 8.0 Hz, 1H), 7.83 (m, 1H), 8.33 (d, J = 7.0 Hz, |
| | 1H), 8.69 (s, 1H), 8.87 (d, J = 6.7 Hz, 1H), 12.45 (s, 1H), 12.97 (s, 1H) |
| 27 | H NMR (400 MHz, DMSO-d6) δ 13.20 (d, J = 6.7 Hz, 1H), 12.68 (s, 1H), |
| | 8.96-8.85 (m, 4H), 8.35 (d, J =7.9 Hz, 1H), 7.91-7.77 (m, 3H), 7.64-7.54 (m, |
| | 3H), 6.82 (m, 1H), 5.05 (s, 0.7H), 4.96 (s, 1.3H), 4.25 (t, J = 5.6 Hz, 1.3H), |
| | 4.00 (t, J = 5.7 Hz, 0.7H), 3.14 (s, 2H), 3.02 (s, 1H), 2.62 (t, J = 5.2 Hz, 2H), |
| | 2.54 (t, J = 5.4 Hz, 1H) |
| 29 | H NMR (400 MHz, CDCl₃) δ 9.0 9 (s, 1H), 8.62 (dd, J = 8.1 and 1.5 Hz, 1H), |
| | 7.83-7.79 (m, 3H), 7.57 (d, J = 7.2 Hz, 1H), 7.38 (t, J = 7.6 Hz, 2H), 7.14 |
| | (t, J = 7.4 Hz, 2H), 5.05 (m, 1H), 1.69 (d, J = 6.6 Hz, 6H) |
| 32 | H NMR (400 MHz, DMSO-d6) δ 12.93 (d, J = 6.6 Hz, 1H), 12.74 (s, 1H), |
| | 11.27 (s, 1H), 8.91 (d, J = 6.7 Hz, 1H), 8.76 (s, 1H), 8.37 (d, J = 8.1 Hz, 1H), |
| | 7.83 (t, J = 8.3 Hz, 1H), 7.77 (d, J = 7.6 Hz, 1H), 7.70 (s, 1H), 7.54 (t, J = 8.1 |
| | Hz, 1H), 7.38 (m, 1H), 6.40 (m, 1H) |
| 33 | H NMR (400 MHz, DMSO-d6) δ 12.92 (s, 1H), 12.47 (s, 1H), 11.08 (s, 1H), |
| | 8.90 (s, 1H), 8.35 (dd, J = 8.1, 1.1 Hz, 1H), 8.20 (t, J = 0.8 Hz, 1H), 7.83 |
| | (t, J = 8.3 Hz, 1H), 7.76 (d, J = 7.7 Hz, 1H), 7.55 (t, J = 8.1 Hz, 1H), 7.50 |
| | (d, J = 8.4 Hz, 1H), 7.30 (t, J = 2.7 Hz, 1H), 7.06 (dd, J = 8.4, 1.8 Hz, 1H), |
| | 6.39 (m, 1H) |
| 35 | H NMR (400 MHz, DMSO-d6) δ 13.01 (d, J = 6.7 Hz, 1H), 12.37 (s, 1H), |
| | 8.86 (d, J = 6.8 Hz, 1H), 8.33 (dd, J = 8.1, 1.3 Hz, 1H), 7.82 (t, J = 8.3 Hz, |
| | 1H), 7.76 (d, J = 8.2 Hz, 1H), 7.54 (t, J = 8.1 Hz, 1H), 7.36 (s, 1H), , 7.19 |
| | (d, J = 8.4 Hz, 1H), 7.08 (d, J= 8.2 Hz, 1H), 3.29 (m, 2H), 1.85 (m, 1H), |
| | 1.73-1.53 (m, 3H), 1.21 (s, 3H), 0.76 (t, J = 7.4 Hz, 3H) |
| 43 | H NMR (400 MHz, DMSO-d6) δ 12.77 (s, 1H), 11.94 (s, 1H), 9.56 (s, 1H), |
| | 8.81 (s, 1H), 8.11 (dd, J = 8.2, 1.1 Hz, 1H), 7.89 (s, 1H), 7.79-7.75 (m, 1H), |
| | 7.70 (d, J = 7.7 Hz, 1H), 7.49-7.45 (m, 1H), 7.31 (t, J = 8.1 Hz, 1H), 7.00 |
| | (s, 1H), 6.93-6.87 (m, 3H), 4.07 (q, J = 7.0 Hz, 2H), 1.38 (s, 9H), 1.28 (t, |
| | J = 7.0 Hz, 3H) |
| 47 | H NMR (400 MHz, DMSO-d6) δ 1.24 (d, J = 6.9 Hz, 6H), 3.00 (m, 1H), 7.55 |
| | (m, 3H), 7.76 (d, J = 7.7 Hz, 1H), 7.83 (m, 1H), 8.26 (d, J = 8.2 Hz, 1H), 8.33 |
| | (d, J = 9.2 Hz, 1H), 8.89 (s, 1H), 12.65 (s, 1H), 12.95 (s, 1H) |
| 56 | H NMR (400 MHz, DMSO-d6) δ 12.81 (d, J = 6.7 Hz, 1H), 12.27 (s, 1H), |
| | 9.62 (s, 1H), 8.82 (d, J = 6.7 Hz, 1H), 8.32 (dd, J = 8.2, 1.3 Hz, 1H), 8.07 |
| | (s, 1H), 7.80 (t, J = 8.4 Hz, 1H), 7.73 (d, J = 7.8 Hz, 1H), 7.52 (t, J = 8.1 Hz, |
| | 1H), 6.58 (s, 1H), 2.62 (m, 4H), 1.71 (m, 4H) |
| 58 | H NMR (400 MHz, DMSO-d6) δ 12.95 (d, J = 6.6 Hz, 1H), 12.39 (s, 1H), |
| | 8.86 (d, J = 6.8 Hz, 1H), 8.33 (d, J = 7.3 Hz, 1H), 7.82 (t, J = 8.3 Hz, 1H), |
| | 7.75 (d, J = 7.8 Hz, 1H), 7.54 (t, J = 8.1 Hz, 1H), 7.29 (d, J = 2.5 Hz, 1H), |
| | 7.07 (dd, J = 8.7, 1.3 Hz, 1H), 6.91 (dd, J = 8.8, 2.5 Hz, 1H), 5.44 (br s, 2H) |
| 64 | H NMR (400 MHz, DMSO-d6) δ 12.92 (s, 1H), 12.41 (s, 1H), 10.63 (s, 1H), |
| | 10.54 (s, 1H), 8.86 (s, 1H), 8.33 (d, J = 8.1 Hz, 1H), 7.82 (t, J = 8.3 Hz, 1H), |
| | 7.76 (d, J = 7.7 Hz, 1H), 7.69 (s, 1H), 7.54 (t, J = 8.1 Hz, 1H), 7.04 (d, J = 8.3 |
| | Hz, 1H), 6.90 (d, J = 8.3 Hz, 1H) |
| 69 | H NMR (400 MHz, DMSO-d6) δ 13.06 (d, J = 6.5 Hz, 1H), 12.51 (s, 1H), |
| | 8.88 (d, J = 6.6 Hz, 1H), 8.33 (dd, J = 8.1, 1.0 Hz, 1H), 7.85-7.74 (m, 3H), |
| | 7.55 (t, J = 8.1 Hz, 1H), 7.38 (dd, J = 8.4, 1.9 Hz, 1H), 7.32 (d, J = 8.5 Hz, 1H), |
| | 3.03 (septet, J = 6.8 Hz, 1H), 1.20 (d, J = 6.7 Hz, 6H) |
| 76 | 1H-NMR (CDCl3, 300 MHz) δ 8.84 (d, J = 6.6 Hz, 1H), 8.31 (d, J = 6.2 Hz, |
| | 1H), 8.01 (d, J = 7.9 Hz, 1H), 7.44-7.13 (m, 8H), 6.78 (d, J = 7.5 Hz, 1H). |
| 77 | H NMR (400 MHz, DMSO-d6) δ 6.40 (m, 1H), 7.36 (t, J = 2.7 Hz, 1H), |
| | 7.43 (d, J = 11.8 Hz, 1H), 7.55 (t, J = 8.1 Hz, 1H), 7.80 (m, 2H), 8.36 (d, |
| | J = 9.2 Hz, 1H), 8.65 (d, J = 6.8 Hz, 1H), 8.91 (s, 1H), 11.19 (s, 1H), 12.72 |
| | (s, 1H), 12.95 (s, 1H) |
| 88 | H NMR (400 MHz, DMSO-d6) δ 12.96 (d, J = 6.6 Hz, 1H), 12.42 (s, 1H), |
| | 8.89 (d, J =6.7 Hz, 1H), 8.33 (dd, J =8.1, 1.2 Hz, 1H), 7.82 (t, J = 8.3 Hz, 1H), |
| | 7.76 (d, J = 7.8 Hz, 1H), 7.66 (d, J = 8.7 Hz, 2H), 7.54 (t, J = 8.1 Hz, 1H), |
| | 7.34 (d, J = 8.7 Hz, 2H), 6.67 (t, J = 6.3 Hz, 1H), 3.12 (d, J = 6.3 Hz, 2H), |
| | 1.35 (s, 9H), 1.22 (s, 6H) |
| 90 | 1H NMR (400 MHz, DMSO-d6) δ 11.98 (s, 1H), 8.89 (s, 1H), 8.34 (dd, J = 8.2, |
| | 1.1 Hz, 1H), 7.84-7.75 (m, 2H), 7.59 (dd, J = 7.8, 1.5 Hz, 1H), 7.55-7.51 |
| | (m, 1H), 7.42 (dd, J = 7.9, 1.5 Hz, 1H), 7.26-7.21 (m, 1H), 7.19-7.14 |
| | (m, 1H), 1.43 (s, 9H) |
| 96 | 1H NMR (400 MHz, DMSO-d6) δ 12.58 (s, 1H), 11.11 (s, 1H), 8.89 (s, 1H), |
| | 8.35 (dd, J = 8.1, 1.1 Hz, 1H), 8.22 (d, J = 1.5 Hz, 1H), 7.83-7.74 (m, 2H), |
| | 7.56-7.51 (m, 2H), 7.30 (d, J = 2.3 Hz, 1H), 7.13 (dd, J = 8.5, 1.8 Hz, 1H), |
| | 4.03 (d, J = 0.5 Hz, 2H) |
| 103 | H NMR (400 MHz, DMSO-d6) δ 1.37 (s, 9H), 1.38 (s, 9H), 7.08 (s, 1H), |
| | 7.17 (s, 1H), 7.74 (m, 1H), 7.86 (m, 1H), 7.98 (dd, J = 9.2, 2.9 Hz, 1H), |
| | 8.90 (d, J = 6.7 Hz, 1H), 9.21 (s, 1H), 11.71 (s, 1H), 13.02 (d, J = 6.7 Hz, 1H) |
| 104 | 1H NMR (400 MHz, DMSO-d6) δ 12.93 (d, J = 6.6 Hz, 1H), 12.41 (s, 1H), |
| | 10.88 (s, 1H), 8.88 (d, J = 6.7 Hz, 1H), 8.36-8.34 (m, 1H), 8.05 (d, J = 0.8 Hz, |
| | 1H), 7.84-7.75 (m, 2H), 7.56-7.52 (m, 1H), 7.35 (d, J = 8.3 Hz, 1H), 7.01 |
| | (dd, J = 8.4, 1.9 Hz, 1H), 6.07-6.07 (m, 1H), 2.37 (s, 3H) |
| 107 | H NMR (400 MHz, DMSO-d6) δ 12.52 (s, 1H), 8.87 (s, 1H), 8.33 (dd, J = 8.2, |
| | 1.1 Hz, 1H), 7.81 (t, J = 8.3 Hz, 1H), 7.75 (d, J = 7.7 Hz, 1H), 7.57-7.51 |
| | (m, 3H), 7.15 (d, J = 8.3 Hz, 1H), 4.51 (s, 2H), 3.56 (t, J = 5.7 Hz, 2H), 2.75 |
| | (t, J = 5.5 Hz, 2H), 1.44 (s, 9H) |
| 109 | H NMR (400 MHz, DMSO-d6) δ 12.97 (br s, 1H), 12.45 (s, 1H), 8.89 (s, 1H), |
| | 8.33 (dd, J = 8.2, 1.1 Hz, 1H), 7.88 (s, 1H), 7.82 (t, J = 8.4 Hz, 1H), 7.75 |
| | (d, J = 7.7 Hz, 1H), 7.54 (t, J = 8.1 Hz, 1H), 7.43 (m, 1H), 7.31 (d, J = 8.5 Hz, |
| | 1H), 4.01 (m, 1H), 3.41 (m, 1H), 2.21 (s, 3H), 1.85 (m, 1H), 1.68-1.51 (m, 3H), |
| | 1.23 (s, 3H), 0.71 (t, J = 7.4 Hz, 3H) |
| 113 | 1H NMR (400 MHz, DMSO-d6) δ 12.92 (d, J = 6.6 Hz, 1H), 12.46 (s, 1H), |
| | 10.72 (d, J = 1.5 Hz, 1H), 8.89 (d, J = 6.7 Hz, 1H), 8.35 (dd, J = 8.1, 1.2 Hz, |
| | 1H), 8.13 (d, J = 1.5 Hz, 1H), 7.84-7.75 (m, 2H), 7.56-7.52 (m, 1H), 7.44 |
| | (d, J = 8.4 Hz, 1H), 7.07-7.04 (m, 2H), 2.25 (d, J = 0.9 Hz, 3H) |
| 114 | 1H NMR (300 MHz, DMSO-d6) δ 12.65 (d, J = 6.9 Hz, 1H), 11.60 (s, 1H), |
| | 9.33 (s, 1H), 8.71 (d, J = 6.6 Hz, 1H), 8.36 (d, J = 1.8 Hz, 1H), 8.03 (d, J = 7.8 |
| | Hz, 1H), 7.66 (t, J = 7.2 Hz, 1H), 7.60 (d, J = 8.1 Hz, 1H), 7.38 (t, J = 7.8 Hz, |
| | 1H), 7.29 (t, J = 7.5 Hz, 1H), 7.12 (m, 2H), 6.97 (m, 3H), 3.97 (m, 2H), 1.45 |
| | (s, 9H), 1.06 (t, J = 6.6 Hz, 3H). |
| 126 | H NMR (400 MHz, DMSO-d6) δ 12.94 (s, 1H), 12.33 (s, 1H), 9.49 (s, 1H), |
| | 8.88 (s, 1H), 8.35 (dd, J = 8.7, 0.5 Hz, 1H), 7.86-7.82 (m, 1H), 7.77 (d, |
| | J = 7.8 Hz, , 7.58-7.54 (m, 1H), 7.40 (d, J = 2.2 Hz, 1H), 7.11 (d, J = 8.5 Hz, |
| | 1H), 6.98 (dd, J = 8.4, 2.2 Hz, 1H), 3.67 (s, 2H), 3.51-3.47 (m, 2H), 3.44-3.41 |
| | (m, 2H), 3.36 (s, 3H), 1.33 (s, 6H) |
| 127 | H NMR (400 MHz, DMSO-d6) δ 1.23 (t, J = 7.0 Hz, 3H), 1.32 (s, 9H), 4.10 |
| | (q, J = 7.0 Hz, 2H), 7.36 (d, J = 8.5 Hz, 1H), 7.54 (m, 3H), 7.76 (d, J = 7.9 Hz, |
| | 1H), 7.82 (m, 1H) 8.33 (d, J = 9.2 Hz, 1H), 8.64 (s, 1H), 8.87 (s, 1H), 12.45 |
| | (s, 1H), 12.99 (s, 1H) |
| 129 | 1H-NMR (CD3OD, 300 MHz) δ 8.83 (s, 1H), 8.41 (d, J = 8.1 Hz, 1H), 7.80 |
| | (m, 2H), 7.65 (d, J = 8.1 Hz, 1H), 7.55 (m, 2H), 7.22 (d, J = 8.1 Hz, 1H), |
| | 3.76 (s, 3H, OMe), 2.62 (q, J = 7.5 Hz, 2H), 1.21 (t, J =7.5 Hz, 3H). |
| 131 | 1H NMR (300 MHz, DMSO-d6) δ 12.37 (s, 1H), 8.81 (s, 1H), 8.30 (d, J = 8.1 |
| | Hz, 1H), 7.77 (m, 2H), 7.52 (t, J = 7.2 Hz, 1H), 7.09 (s, 1H), 6.74 (s, 1H), |
| | 6.32 (s, 1H), 5.47 (s, 2H). |
| 135 | 1H-NMR (CDCl3, 300 MHz) δ 8.86 (d, J = 6.6 Hz, 1H), 8.32 (d, J = 6.2 Hz, |
| | 1H), 8.07 (d, J = 7.9 Hz, 1H), 7.47-7.24 (m, 6H), 6.95-6.83 (m, 3H), 5.95 |
| | (s, 2H). |
| 136 | H NMR (400 MHz, DMSO-d6) δ 1.29 (s, 9H), 1.41 (s, 9H), 7.09 (d, J = 2.4 Hz, |
| | 1H), 7.47 (d, J = 2.3 Hz, 1H), 7.57 (t, J = 8.1 Hz, 1H), 7.77 (d, J = 7.8 Hz, 1H), |
| | 7.85 (t, J = 8.4 Hz, 1H), 8.36 (d, J = 9.5 Hz, 1H), 8.93 (d, J = 6.8 Hz, 1H), 9.26 |
| | (s, 1H), 12.66 (s, 1H), 13.04 (d, J = 6.6 Hz, 1H) |
| 141 | H NMR (400 MHz, DMSO-d6) δ 12.96 (d, J = 6.6 Hz, 1H), 12.42 (s, 1H), |
| | 8.87 (d, J = 6.8 Hz, 1H), 8.33 (dd, J = 8.1, 1.2 Hz, 1H), 7.85-7.75 (m, 3H), |
| | 7.55 (t, J = 8.1 Hz, 1H), 7.46 (dd, J = 8.2, 2.2 Hz, 1H), 7.16 (d, J = 8.5 Hz, |
| | 1H), 4.14 (q, J = 7.1 Hz, 2H), 2.18 (s, 3H), 1.27 (t, J = 7.1 Hz, 3H) |
| 143 | H NMR (400 MHz, DMSO-d6) δ 12.96 (d, J = 6.8 Hz, 1H), 12.56 (s, 1H), |
| | 9.44 (s, 1H), 8.87 (d, J = 6.8 Hz, 1H), 8.34 (dd, J = 8.2, 1.3 Hz, 1H), 8.08 |
| | (d, J = 7.4 Hz, 1H), 7.83 (t, J = 8.3 Hz, 1H), 7.76 (d, J = 7.7 Hz, 1H), 7.55 |
| | (t, J = 8.1 Hz, 1H), 7.00 (d, J = 13.3 Hz, 1H), 1.34 (s, 9H) |
| 150 | 1H-NMR (DMSO d6, 300 MHz) δ 8.86 (d, J = 6.9 Hz, 1H), 8.63 (s, 1H), |
| | 8.30 (d, J = 8.1 Hz, 1H), 7.86 (d, J = 8.7 Hz, 2H), 7.82-7.71 (m, 2H), 7.64 |
| | (d, J = 8.4 Hz, 2H), 7.52 (td, J = 1.2Hz, 1H). |
| 157 | 1H-NMR (CD3OD, 300 MHz) δ 8.91 (s, 1H), 8.57 (s, 1H), 8.45 (d, J = 8.3 Hz, |
| | 1H), 7.83 (t, J = 7.2 Hz, 1H), 7.69 (d, J = 9.0 Hz, 1H), 7.57 (t, J = 7.9 Hz, 1H), |
| | 7.46 (d, J = 8.5 Hz, 1H), 7.16 (d, J = 6.0 Hz, 1H), 3.08 (s, 3H, NMe), 2.94 |
| | (q, J = 7.4 Hz, 2H), 1.36 (t, J = 7.4 Hz, 3H). |
| 161 | H NMR (400 MHz, DMSO-d6) δ 12.96 (s, 1H), 12.41 (s, 1H), 8.88 (s, 1H), |
| | 8.33 (dd, J =8.2, 1.2 Hz, 1H), 7.84-7.80 (m, 1H), 7.75 (d, J =7.9 Hz, 1H), |
| | 7.55 (t, J = 8.1 Hz, 1H), , 7.44 (s, 1H), 7.19 (s, 2H), 4.13 (t, J = 4.6 Hz, 2H), |
| | 3.79 (t, J = 4.6 Hz, 2H), 3.54 (q, J = 7.0 Hz, 2H), 1.36 (s, 9H), 1.15 (t, J = 7.0 |
| | Hz, 3H) |
| 163 | 1H-NMR (300 MHz, DMSO-d6) δ 12.87 (d, J = 6.3 Hz, 1H), 11.83 (s, 1H), |
| | 8.76 (d, J = 6.3 Hz, 1H), 8.40 (s, 1H), 8.26 (br s, 2H), 8.08 (dd, J = 8.4 Hz, |
| | J = 1.5 Hz, 1H), 7.75 (m, 1H), 7.67 (d, J = 7.8 Hz, 1H), 7.47-7.37 (m, 2H), 7.24 |
| | (d, J = 0.9 Hz, 1H), 7.15 (dd, J = 7.5 Hz, J = 1.8 Hz, 1H), 7.10 (d, J = 8.1 Hz, |
| | 1H), 7.02 (dt, J = 7.5 Hz, J = 0.9 Hz, 1H), 4.07 (m, 4H), 1.094 (t, J = 6.9 Hz, |
| | 3H). |
| 167 | H NMR (400 MHz, DMSO-d6) δ 2.03 (s, 3H), 4.91 (s, 2H), 6.95 (m, 3H), |
| | 7.53 (m, 1H), 7.75 (d, J = 8.2 Hz, 1H), 7.81 (m, 1H), 8.33 (d, J = 8.0 Hz, 1H), |
| | 8.84 (s, 1H), 12.20 (s, 1H), 12.90 (s, 1H) |
| 169 | 1H NMR (400 MHz, DMSO-d6) δ 12.94 (d, J = 5.3 Hz, 1H), 12.51 (s, 1H), |
| | 8.89 (d, J = 6.3 Hz, 1H), 8.36 (dd, J = 8.1, 1.1 Hz, 1H), 8.06 (t, J = 0.7 Hz, 1H), |
| | 7.85-7.75 (m, 2H), 7.57-7.51 (m, 2H), 7.28 (d, J = 3.1 Hz, 1H), 7.24 |
| | (dd, J = 8.4, 1.8 Hz, 1H), 6.39 (dd, J = 3.1, 0.8 Hz, 1H), 3.78 (s, 3H) |
| 178 | 1H NMR (400 MHz, DMSO-d6) δ 12.86 (s, 1H), 8.89 (d, J = 6.8 Hz, 1H), |
| | 8.65 (dd, J = 8.1, 1.6 Hz, 1H), 8.19 (dd, J = 8.2, 1.3 Hz, 1H), 7.80-7.71 (m, 2H), |
| | 7.48-7.44 (m, 2H), 7.24-7.20 (m, 1H), 7.16-7.09 (m, 2H), 7.04-7.00 (m, 1H), |
| | 6.80 (dd, J = 8.0, 1.3 Hz, 1H), 6.69 (dd, J = 8.1, 1.4 Hz, 1H), 1.45 (s, 9H) |
| 183 | 1H NMR (400 MHz, DMSO-d6) δ 12.42 (s, 1H), 8.88 (s, 1H), 8.33 (dd, |
| | J = 8.2, 1.1 Hz, 1H), 8.06 (d, J = 2.1 Hz, 1H), 7.84-7.75 (m, 2H), 7.56-7.52 |
| | (m, 1H), 7.38 (dd, J = 8.2, 2.1 Hz, 1H), 7.08 (d, J = 8.3 Hz, 1H), 3.66-3.63 |
| | (m, 2H), 2.70 (t, J = 6.5 Hz, 2H), 1.86-1.80 (m, 2H), 1.51 (s, 9H) |
| 186 | H NMR (400 MHz, DMSO-d6) δ 12.93 (s, 1H), 12.47 (s, 1H), 10.72 (s, 1H), |
| | 8.89 (s, 1H), 8.35 (dd, J = 8.2, 1.1 Hz, 1H), 8.13 (d, J = 1.6 Hz, 1H), 7.82 |
| | (t, J = 8.2 Hz, 1H), 7.76 (d, J = 7.8 Hz, 1H), 7.54 (t, J = 7.5 Hz, 1H), 7.50 |
| | (d, J = 8.4 Hz, 1H), 7.05-7.02 (m, 2H), 3.19 (quintet, J = 8.2 Hz, 1H), 2.08 |
| | (m, 2H), 1.82-1.60 (m, 6H) |
| 187 | 1H NMR (400 MHz, DMSO-d6) δ 12.63 (s, 1H), 8.91 (s, 1H), 8.87-8.87 |
| | (m, 1H), 8.36 (dd, J = 8.2, 1.2 Hz, 1H), 7.85-7.75 (m, 3H), 7.64-7.53 (m, 3H), |
| | 6.71 (dd, J = 3.7, 0.5 Hz, 1H), 2.67 (s, 3H) |
| 188 | H NMR (400 MHz, DMSO-d6) δ 12.84 (s, 1H), 12.73 (d, J = 6.6 Hz, 1H), |
| | 11.39 (s, 1H), 8.85 (d, J = 6.7 Hz, 1H), 8.61 (s, 1H), 8.33 (d, J = 6.8 Hz, 1H), |
| | 8.23 (s, 1H), 7.80 (t, J = 8.4 Hz, 1H), 7.73 (d, J = 7.8 Hz, 1H), 7.52 (t, J = 8.1 |
| | Hz, 1H), 7.43 (m, 1H), 6.54 (m, 1H), 4.38 (q, J = 7.1 Hz, 2H), 1.36 |
| | (t, J = 7.1 Hz, 3H) |
| 204 | H NMR (400 MHz, DMSO-d6) δ 12.97 (s, 1H), 12.37 (s, 1H), 8.87 (d, J = 1.2 |
| | Hz, 1H), 8.32 (d, J = 8.2 Hz, 1H), 7.82 (dd, J = 8.2, 7.0 Hz, 1H), 7.75 |
| | (d, J = 8.3 Hz, 1H), 7.54 (t, J = 7.5 Hz, 1H), 7.32-7.28 (m, 2H), 7.05 |
| | (d, J = 8.4 Hz, 1H), 4.16 (t, J = 4.9 Hz, 2H), 1.78 (t, J = 4.9 Hz, 2H), 1.29 |
| | (s, 6H), |
| 207 | H NMR (400 MHz, DMSO-d6) δ 12.92 (br s, 1H), 12.50 (s, 1H), 10.95 |
| | (s, 1H), 8.89 (s, 1H), 8.35 (dd, J = 8.2, 1.1 Hz, 1H), 8.17 (d, J = 1.5 Hz, |
| | 1H), 7.82 (t, J = 8.3 Hz, 1H), 7.76 (d, J = 7.7 Hz, 1H), 7.55 (t, J = 8.1 Hz, |
| | 1H), 7.46 (d, J = 8.4 Hz, IH), 7.21 (d, J = 2.3 Hz, 1H), 7.06 (dd, J = 8.5, |
| | 1.8 Hz, 1H), 4.09 (q, J = 7.1 Hz, 2H), 3.72 (s, 2H), 1.20 (t, J = 7.1 Hz, 3H) |
| 215 | H NMR (400 MHz, DMSO-d6) δ 12.97 (s, 1H), 12.50 (s, 1H), 8.89 (s, |
| | 1H), 8.34 (dd, J = 8.1, 1.1 Hz, 1H), 7.83 (t, J = 8.3 Hz, 1H), 7.75 (m, 3H), |
| | 7.55 (t, J = 8.1 Hz, 1H), 7.37 (t, J = 7.9 Hz, 2H), 7.10 (t, J = 6.8 Hz, 1H) |
| 220 | H NMR (400 MHz, DMSO-d6) δ 12.99 (d, J = 6.6 Hz, 1H), 12.07 (s, 1H), |
| | 8.93 (d, J = 6.8 Hz, 1H), 8.35 (d, J = 7.1 Hz, 1H), 8.27 (s, 1H), 8.12 (s, 1H), |
| | 7.85-7.77 (m, 2H), 7.54 (td, J = 7.5, 1.2 Hz, 1H), 6.81 (s, 1H), 1.37 (d, |
| | J = 3.9 Hz, 9H), 1.32 (d, J = 17.1 Hz, 9H) |
| 225 | 1H NMR (CD3OD, 300 MHz) δ 8.79 (s, 1H), 8.37 (d, J = 7.9 Hz, 1H), 7.75 |
| | (m, 2H), 7.61 (d, J = 8.3 Hz, 1H), 7.5 (m, 2H), 7.29 (d, J = 8.3 Hz, 1H), |
| | 4.21 (q, J = 7.2, 2H), 3.17 (m, 1H), 1.32 (t, J = 7.2 Hz, 3H), 1.24 (d, J = 6.9 |
| | Hz, 6H). |
| 232 | 1H-NMR (CD3OD, 300 MHz) δ 8.87 (s, 1H), 8.45 (d, J = 8.25, 1H), 8.27 |
| | (m, 1H), 7.83 (t, J = 6.88, 1H), 7.67 (d, J = 8.25, 1H), 7.54 (t, J = 7.15, 1H), |
| | 7.39 (d, J = 6.05, 1H), 7.18 (d, J = 8.5, 1H), 2.77 (t, J = 6.87, 2H), 2.03 (s, 3H), |
| | 1.7 (q, 2H), 1.04 (t, J = 7.42, 3H) |
| 233 | 1H NMR (400 MHz, DMSO-d6) δ 12.75 (d, J = 13.6 Hz, 1H), 8.87 (s, 1H), |
| | 8.32-8.28 (m, 2H), 7.76-7.70 (m, 2H), 7.60 (d, J = 7.8 Hz, 1H), 7.49-7.45 |
| | (m, 1H), 7.18 (d, J = 8.4 Hz, 1H), 4.11 (t, J = 8.3 Hz, 2H), 3.10 (t, J = 7.7 Hz, |
| | 2H), 2.18 (s, 3H) |
| 234 | 1H NMR (400 MHz, DMSO-d6) δ 12.49 (s, 1H), 11.50 (s, 1H), 8.90 (s, 1H), |
| | 8.36-8.34 (m, 2H), 7.97 (s, 1H), 7.85-7.81 (m, 1H), 7.77-7.75 (m, 1H), |
| | 7.56-7.50 (m, 2H), 6.59-6.58 (m, 1H) |
| 235 | H NMR (400 MHz, DMSO-d6) δ 13.09 (d, J = 6.5 Hz, 1H), 12.75 (s, 1H), |
| | 9.04 (s, 1H), 8.92 (d, J = 6.8 Hz, 1H), 8.42 (d, J = 7.1 Hz, 1H), 8.34 |
| | (d, J = 6.9 Hz, 1H), 7.85 (t, J = 8.4 Hz, 1H), 7.78 (d, J = 7.7 Hz, 1H), |
| | 7.63-7.56 (m, 2H), 3.15 (m, 1H), 1.29 (d, J = 6.9 Hz, 6H) |
| 238 | H NMR (400 MHz, DMSO-d6) δ 12.93 (d, J = 6.4 Hz, 1H), 12.29 (s, 1H), |
| | 8.85 (d, J = 6.7 Hz, 1H), 8.32 (d, J = 8.1 Hz, 1H), 7.82 (t, J = 8.3 Hz, 1H), |
| | 7.75 (d, J = 7.9 Hz, 1H), 7.54 (t, J = 8.1 Hz, 1H), 7.17 (m, 2H), 6.94 (m, 1H), |
| | 3.79 (m, 2H), 3.21-2.96 (m, 4H), 1.91-1.76 (m, 4H), 1.52 (m, 2H), 1.43 (s, 9H) |
| 242 | H NMR (400 MHz, DMSO-d6) δ 12.95 (d, J = 6.6 Hz, 1H), 12.65 (s, 1H), |
| | 8.87 (d, J = 6.8 Hz, 1H), 8.34 (dd, J = 8.1, 1.1 Hz, 1H), 8.17 (s, 1H), 7.83 |
| | (t, J = 8.3 Hz, 1H), 7.76 (d, J = 7.8 Hz, 1H), 7.54 (t, J = 8.1 Hz, 1H), 7.37 |
| | (s, 1H), 5.60 (s, 2H) |
| 243 | 1H-NMR (CD3OD, 300 MHz) δ 8.87 (s, 1H), 8.45 (d, J = 8.25, 1H), 8.27 |
| | (m, 1H), 7.83 (t, J = 6.88, 1H), 7.67 (d, J = 8.25, 1H), 7.54 (t, J = 7.15, 1H), |
| | 7.39 (d, J = 6.05, 1H), 7.18 (d, J = 8.5, 1H), 2.77 (t, J = 6.87, 2H), 2.03 |
| | (s, 3H), 1.7 (q, 2H), 1.04 (t, J = 7.42, 3H) NMR Shows regio isomer |
| 244 | H NMR (400 MHz, DMSO-d6) δ 12.89 (s, 1H), 12.42 (s, 1H), 10.63 (s, 1H), |
| | 8.88 (d, J = 6.7 Hz, 1H), 8.35 (d, J = 8.2 Hz, 1H), 8.03 (d, J = 1.6 Hz, 1H), |
| | 7.82 (t, J = 8.3 Hz, 1H), 7.76 (d, J = 7.7 Hz, 1H), 7.54 (t, J = 8.1 Hz, 1H), |
| | 7.29 (d, J = 8.3 Hz, 1H), 7.02 (dd, J = 8.4, 1.8 Hz, 1H), 2.69 (t, J = 5.3 Hz, 2H), |
| | 2.61 (t, J = 5.0 Hz, 2H), 1.82 (m, 4H) |
| 248 | H NMR (400 MHz, DMSO-d6) δ 12.95 (d, J = 6.6 Hz, 1H), 12.42 (s, 1H), |
| | 9.30 (s, 1H), 8.86 (d, J = 6.8 Hz, 1H), 8.33 (dd, J = 8.1, 1.3 Hz, 1H), |
| | 7.85-7.81 (m, 2H), 7.76 (d, J = 7.8 Hz, 1H), 7.55 (t, J = 8.1 Hz, 1H), 7.49 |
| | (dd, J = 8.2, 2.2 Hz, 1H), 7.18 (d, J = 8.3 Hz, 1H), 2.18 (s, 3H), 2.08 (s, 3H) |
| 259 | H NMR (400 MHz, DMSO-d6) δ 0.86 (t, J = 7.4 Hz, 3H), 1.29 (d, J = 6.9 Hz, |
| | 3H), 1.67 (m, 2H), 2.88 (m, 1H), 7.03 (m, 2H), 7.53 (m, 2H), 7.80 (m, 2H), |
| | 8.13 (s, 1H), 8.35 (d, J = 8.2 Hz, 1H), 8.89 (s, 1H), 10.75 (s, 1H), 12.45 (s, 1H), |
| | 12.84 (s, 1H) |
| 260 | H NMR (400 MHz, DMSO-d6) δ 13.23 (d, J = 6.6 Hz, 1H), 12.20 (s, 1H), |
| | 10.22 (br s, 2H), 8.88 (d, J = 6.8 Hz, 1H), 8.34 (d, J = 7.8 Hz, 1H), 7.86-7.80 |
| | (m, 3H), 7.56-7.52 (m, 2H), 7.15 (dd, J = 8.5, 2.4 Hz, 1H), 1.46 (s, 9H) |
| 261 | 1H-NMR (d6-DMSO, 300 MHz) δ 11.99 (s, 1H, NH), 8.76 (s, J = 6.6 Hz, 1H), |
| | 8.26 (d, J = 6.2 Hz, 1H), 8.09 (d, J = 7.9 Hz, 1H), 7.72-7.63 (m, 2H), |
| | 7.44-7.09 (m, 7H), 2.46 (s, 3H), 2.25 (s, 3H). |
| 262 | 1H NMR (400 MHz, DMSO-d6) δ 13.00 (s, 1H), 12.53 (s, 1H), 10.62 (s, 1H), |
| | 8.88 (s, 1H), 8.33 (dd, J = 8.2, 1.2 Hz, 1H), 7.85-7.75 (m, 2H), 7.57-7.50 |
| | (m, 2H), 7.34-7.28 (m, 2H), 3.46 (s, 2H) |
| 266 | H NMR (400 MHz, DMSO-d6) δ 12.94 (d, J = 6.6 Hz, 1H), 12.57 (s, 1H), |
| | 10.37 (s, 1H), 8.88 (d, J = 6.8 Hz, 1H), 8.34-8.32 (m, 1H), 7.99 (s, 1H), |
| | 7.85-7.81 (m, 1H), 7.76 (d, J = 7.8 Hz, 1H), 7.56-7.52 (m, 1H), 7.38 (s, 1H), |
| | 1.37 (s, 9H) |
| 268 | H NMR (400 MHz, DMSO-d6) δ 13.02 (s, 1H), 12.62 (s, 1H), 8.91 (s, 1H), |
| | 8.34 (dd, J = 8.1, 1.1 Hz, 1H), 8.22 (d, J = 2.4 Hz, 1H), 8.14 (dd, J = 8.8, 2.4 |
| | Hz, 1H), 7.84 (t, J = 8.3 Hz, 1H), 7.77 (d, J = 7.8 Hz, 1H), 7.65-7.54 (m, 4H), |
| | 1.52 (s, 9H) |
| 271 | H NMR (400 MHz, DMSO-d6) δ 1.38 (s, 9H), 4.01 (s, 2H), 7.35 (s, 2H), |
| | 7.55 (m, 1H), 7.65 (s, 1H), 7.79 (d, J = 8.2 Hz, 1H), 7.83 (m, 1H), 8.33 |
| | (d, J = 7.6 Hz, 1H), 8.86 (d, J = 6.8 Hz, 1H), 12.49 (s, 1H), 13.13 (s, 1H) |
| 272 | 1H-NMR (d6-Acetone, 300 MHz) δ 8.92 (d, J = 6.6 Hz, 1H), 8.39 (d, J = 7.8 Hz, |
| | 1H), 7.94 (s, 1H), 7.79 (s, 1H), 7.77 (s, 2H), 7.53 (m, 1H), 7.36 (s, 1H), |
| | 3.94-3.88 (m, 5H), 3.64-3.59 (m, 3H), 3.30 (m, 4H). |
| 274 | H NMR (400 MHz, DMSO-d6) δ 13.21 (d, J = 6.6 Hz, 1H), 11.66 (s, 1H), |
| | 10.95 (s, 1H), 9.00 (d, J = 6.5 Hz, 1H), 8.65 (d, J = 2.1 Hz, 1H), 8.18 |
| | (dd, J = 8.7, 2.2 Hz, 1H), 7.97 (d, J = 8.8 Hz, 1H), 7.57 (m, 2H), 7.31 |
| | (t, J = 2.7 Hz, 1H), 6.40 (t, J = 2.0 Hz, 1H), 3.19 (m, 4H), 1.67 (m, 4H), |
| | 1.46 (s, 9H) |
| 275 | H NMR (400 MHz, DMSO-d6) δ 12.23 (s, 1H), 9.47 (s, 1H), 9.20 (s, 1H), |
| | 8.43 (d, J = 7.9 Hz, 1H), 7.79 (t, J = 2.0 Hz, 2H), 7.56 (m, 1H), 7.38-7.26 |
| | (m, 6H), 7.11 (d, J = 8.4 Hz, 1H), 6.99 (dd, J = 8.4, 2.1 Hz, 1H), 5.85 (s, 2H), |
| | 1.35 (s, 9H) |
| 282 | 1H NMR (CD3OD, 300 MHz) δ 8.90 (s, 1H), 8.51 (s, 1H), 8.44 (d, J = 7.9 Hz, |
| | 1H), 7.82 (t, J = 8.3 Hz, 1H), 7.69 (d, J = 8.5 Hz, 1H), 7.56 (t, J = 7.7 Hz, 2H), |
| | 7.42 (d, J = 7.9 Hz, 1H), 7.07 (d, J = 5.8 Hz, 1H), 2.93 (q, J = 7.4 Hz, 2H), |
| | 1.36 (t, J = 7.5 Hz, 3H). |
| 283 | 1H-NMR (CDCl3, 300 MHz) δ 8.82 (d, J = 6.6 Hz, 1H), 8.29 (d, J = 6.2 Hz, |
| | 1H), 8.06 (d, J = 7.9 Hz, 1H), 7.43-7.24 (m, 6H), 7.02 (m, 2H), 6.87-6.81 |
| | (dd, 2H), 3.76 (s, 3H). |
| 287 | H NMR (400 MHz, DMSO-d6) δ 13.51 (s, 1H), 13.28 (d, J = 6.6 Hz, 1H), |
| | 11.72 (d, J = 2.2 Hz, 1H), 9.42 (s, 1H), 8.87 (d, J = 6.9 Hz, 1H), 8.04 |
| | (d, J = 7.4 Hz, 1H), 7.67 (t, J = 8.2 Hz, 1H), 7.17 (dd, J = 8.3, 0.8 Hz, 1H), |
| | 7.01 (d, J = 13.7 Hz, 1H), 6.81 (dd, J = 8.1, 0.8 Hz, 1H), 2.10 (m, 2H), |
| | 1.63-1.34 (m, 8H), 1.26 (s, 3H) |
| 288 | H NMR (400 MHz, DMSO-d6) δ 13.16 (s, 1H), 12.85 (s, 1H), 8.98 (s, 1H), |
| | 8.43 (dd, J = 8.1, 1.1 Hz, 1H), 8.34 (dd, J = 10.3, 3.1 Hz, 1H), 7.93 |
| | (t, J = 8.4 Hz, 1H), 7.86 (d, J = 7.7 Hz, 1H), 7.66 (t, J = 8.1 Hz, 1H), 7.03 |
| | (dd, J = 10.7, 3.2 Hz, 1H), 4.06 (s, 3H), 1.42 (s, 9H) |
| 295 | H NMR (400 MHz, DMSO-d6) δ 1.98 (m, 4H), 3.15 (m, 4H), 7.04 (m, 2H), |
| | 7.17 (d, J = 7.8 Hz, 1H), 7.52 (m, 1H), 7.74 (d, J = 7.8 Hz, 1H), 7.81 |
| | (m, 1H), 8.19 (dd, J = 7.9, 1.4 Hz, 1H), 8.33 (d, J = 8.1 Hz, 1H), 8.88 |
| | (d, J = 6.7 Hz, 1H), 12.19 (s, 1H), 12.87 (s, 1H) |
| 299 | 1H NMR (400 MHz, DMSO-d6) δ 12.93-12.88 (m, 1H), 12.18 (s, 1H), |
| | 8.83 (d, J = 6.8 Hz, 1H), 8.38-8.31 (m, 1H), 7.85-7.67 (m, 2H), 7.57-7.51 |
| | (m, 1H), 6.94 (s, 1H), 6.81-6.74 (m, 2H), 3.19-3.16 (m, 2H), 2.68-2.61 (m, 2H), |
| | 1.80-1.79 (m, 2H) |
| 300 | H NMR (400 MHz, DMSO-d6) δ 13.23 (d, J = 6.6 Hz, 1H), 12.59 (s, 1H), |
| | 8.87 (d, J =6.8 Hz, 1H), 8.33 (d, J = 7.7 Hz, 1H), 7.86-7.79 (m, 3H), |
| | 7.58-7.42 (m, 3H), 3.38 (m, 2H), 1.88 (m, 2H), 1.30 (s, 6H) |
| 303 | H NMR (400 MHz, DMSO-d6) δ 12.96 (d, J = 6.5 Hz, 1H), 12.47 (s, 0.4H), |
| | 12.43 (s, 0.6H), 8.87 (dd, J = 6.7, 2.3 Hz, 1H), 8.33 (d, J = 8.1 Hz, 1H), 7.82 |
| | (t, J = 8.2 Hz, 1H), 7.75 (d, J = 8.3 Hz, 1H), 7.62-7.52 (m, 3H), 7.17 |
| | (d, J = 8.3 Hz, 1H), 4.66 (s, 0.8H), 4.60 (s, 1.2H), 3.66 (t, J = 5.9 Hz, 2H), |
| | 2.83 (t, J = 5.8 Hz, 1.2H), 2.72 (t, J = 5.9 Hz, 0.8H), 2.09 (m, 3H) |
| 304 | 1H NMR (300 MHz, DMSO-d6) δ 11.70 (s, 1H), 8.74 (s, 1H), 8.15 (s, 1H), |
| | 8.07 (m, 1H), 7.72 (m, 1H), 7.63 (d, J = 8.4 Hz, 1H), 7.45-7.31 (m, 3H), |
| | 7.15-6.95 (m, 5H), 4.17 (d, J = 6.0 Hz, 2H), 4.02 (q, J = 6.9 Hz, 2H), 1.40 |
| | (s, 9H), 1.09 (t, J = 6.9 Hz, 3H). |
| 307 | 1H-NMR (CDCl3, 300 MHz) δ 8.81 (d, J = 6.6 Hz, 1H), 8.30 (d, J = 6.2 Hz, |
| | 1H), 8.02 (d, J = 7.9 Hz, 1H), 7.44-7.26 (m, 9H), 6.79 (d, J = 7.5 Hz, 1H). |
| 318 | 1H-NMR (d6-Acetone, 300 MHz) δ 8.92 (bs, 1H), 8.40 (d, J = 8.1 Hz, 1H), |
| | 8.05 (bs, 1H), 7.94 (bs, 1H), 7.78 (bs, 2H), 7.52 (m, 1H), 7.36 (bs, 1H), 3.97 |
| | (t, J = 7.2 Hz, 2H), 3.66 (t, J = 8 Hz, 2H), 3.31-3.24 (m, 6H), 1.36-1.31 (m, 4H). |
| 320 | ¹H NMR (400 MHz, DMSO-d6) δ 12.90 (s, 1H), 12.44 (s, 1H), 10.86 (s, 1H), |
| | 8.90 (s, 1H), 8.35 (dd, J = 8.2, 1.0 Hz, 1H), 8.12 (t, J = 0.8 Hz, 1H), |
| | 7.84-7.75 (m, 2H), 7.56-7.52 (m, 1H), 7.37 (d, J = 8.3 Hz, 1H), 6.99 (dd, |
| | J = 8.4, 1.9 Hz, 1H), 6.08-6.07 (m, 1H), 1.35 (s, 9H) |
| 321 | H NMR (400 MHz, DMSO-d6) δ 2.93 (m, 4H), 3.72 (m, 4H), 7.10 (m, 2H), |
| | 7.27 (d, J = 7.8 Hz, 1H), 7.51 (m, 6H), 7.74 (d, J = 8.2 Hz, 1H), 7.81 (m, 1H), |
| | 8.40 (d, J = 8.1 Hz, 1H), 8.58 (d, J = 8.0 Hz, 1H), 8.88 (d, J = 6.7 Hz, 1H), |
| | 12.69 (s, 1H), 12.86 (s, 1H) |
| 323 | H NMR (400 MHz, DMSO-d6) δ 12.94 (br s, 1H), 12.44 (s, 1H), 8.89 (s, 1H), |
| | 8.33 (dd, J = 8.2, 1.1 Hz, 1H), 7.82 (t, J = 8.3 Hz, 1H), 7.76 (d, J = 7.7 Hz, 1H), |
| | 7.67 (d, J = 8.8 Hz, 2H), 7.54 (t, J = 8.1 Hz, 1H), 7.35 (d, J = 8.7 Hz, 2H), |
| | 7.02 (t, J = 6.3 Hz, 1H), 3.50 (s, 3H), 3.17 (d, J = 6.2 Hz, 2H), 1.23 (s, 6H) |
| 334 | H NMR (400 MHz, DMSO-d6) δ 13.02 (br s, 1H), 12.46 (s, 1H), 8.89 (s, 1H), |
| | 8.33 (dd, J = 8.2, 1.1 Hz, 1H), 7.89 (s, 1H), 7.82 (t, J = 8.3 Hz, 1H), 7.76 |
| | (d, J = 7.8 Hz, 1H), 7.55 (t, J = 8.1 Hz, 1H), 7.44 (m, 1H), 7.37 (d, J = 8.6 Hz, |
| | 1H), 3.85 (m, 2H), 3.72 (t, J = 6.0 Hz, 2H), 3.18-3.14 (m, 2H), 2.23 (s, 3H), |
| | 1.93 (t, J = 5.7 Hz, 2H), 1.79 (m, 2H), 1.53 (m, 2H), 1.43 (s, 9H) |
| 337 | H NMR (400 MHz, DMSO-d6) δ 12.19 (s, 1H), 9.35 (s, 1H), 8.22 (dd, J = 8.1, |
| | 1.1 Hz, 1H), 8.08 (s, 1H), 7.74-7.70 (m, 1H), 7.65 (d, J = 7.8 Hz, 1H), |
| | 7.44-7.40 (m, 1H), 7.23 (s, 1H), 3.31 (s, 3H), 1.37 (s, 9H), 1.36 (s, 9H) |
| 351 | 1H NMR (400 MHz, DMSO-d6) δ 12.92 (s, 1H), 12.34 (s, 1H), 10.96 (s, 1H), |
| | 8.91 (s, 1H), 8.48 (s, 1H), 8.37 (d, J = 8.1 Hz, 1H), 7.84-7.76 (m, 2H), |
| | 7.53 (t, J = 7.4 Hz, 1H), 7.39 (s, 1H), 7.26 (t, J = 2.6 Hz, 1H), 6.34 (s, 1H), |
| | 2.89-2.84 (m, 2H), 1.29 (t, J = 7.4 Hz, 3H) |
| 353 | 1H NMR (400 MHz, DMSO-d6) δ 11.90 (s, 1H), 9.30 (s, 1H), 8.88 (s, 1H), |
| | 8.34 (dd, J = 8.2, 1.1 Hz, 1H), 7.84-7.71 (m, 3H), 7.55-7.50 (m, 1H), |
| | 7.28-7.26 (m, 1H), 7.20-7.17 (m, 1H), 1.47 (s, 9H), 1.38 (s, 9H) |
| 356 | 1H-NMR (CD3OD, 300 MHz) δ 8.89 (s, 1H), 8.59 (s, 1H), 8.45 (d, J = 8.3 Hz, |
| | 1H), 7.83 (t, J = 7.2 Hz, 1H), 7.69 (d, J = 9.0 Hz, 1H), 7.57 (t, J = 7.9 Hz, 1H), |
| | 7.42 (d, J = 8.5 Hz, 1H), 7.17 (d, J = 6.0 Hz, 1H), 3.09 (s, 3H, NMe), 2.91 |
| | (t, J = 7.4 Hz, 2H), 1.76 (m, 2H), 1.09 (t, J = 7.4 Hz, 3H). |
| 357 | H NMR (400 MHz, DMSO-d6) δ 12.91 (d, J = 6.6 Hz, 1H), 12.45 (s, 1H), |
| | 10.73 (d, J = 1.9 Hz, 1H), 8.89 (d, J = 6.7 Hz, 1H), 8.35 (dd, J = 8.1, 1.3 Hz, |
| | 1H), 8.13 (d, J = 1.6 Hz, 1H), 7.83 (t, J = 8.3 Hz, 1H), 7.76 (d, J = 7.7 Hz, |
| | 1H), 7.57-7.51 (m, 2H), 7.06-7.02 (m, 2H), 3.12 (septet, J = 6.6 Hz, 1H), |
| | 1.31 (d, J = 6.9 Hz, 6H) |
| 363 | 1H-NMR (CDCl3, 300 MHz) δ 8.86 (d, J = 6.6 Hz, 1H), 8.24 (d, J = 6.2 Hz, |
| | 1H), 8.14 (d, J = 7.9 Hz, 1H), 7.43-7.16 (m, 5H), 7.02-6.92 (m, 2H), 6.83 |
| | (d, J = 7.9 Hz, 2H), 3.87 (s, 3H). |
| 368 | H NMR (400 MHz, DMSO-d6) δ 12.97 (d, J = 6.6 Hz, 1H), 12.36 (s, 1H), |
| | 8.86 (d, J = 6.7 Hz, 1H), 8.33 (dd, J = 8.1, 1.0 Hz, 1H), 7.83 (t, J = 8.3 Hz, 1H), |
| | 7.76 (d, J = 7.8 Hz, 1H), 7.62 (s, 1H), 7.55 (t, J = 8.1 Hz, 1H), 7.25 (dd, J = 8.7, |
| | 2.2 Hz, 1H), 7.01 (d, J = 8.8 Hz, 1H), 3.98 (t, J = 6.5 Hz, 2H), 1.78 (sextet, |
| | J = 6.9 Hz, 2H), 1.02 (t, J = 7.4 Hz, 3H) |
| 375 | H NMR (400 MHz, DMSO-d6) δ 12.93 (d, J = 6.2 Hz, 1H), 12.35 (s, 1H), |
| | 8.86 (d, J = 6.7 Hz, 1H), 8.33 (d, J = 6.9 Hz, 1H), 7.82 (t, J = 8.3 Hz, 1H), |
| | 7.75 (d, J = 7.8 Hz, 1H), 7.54 (t, J = 8.1 Hz, 1H), 7.47-7.43 (m, 2H), 7.04 |
| | (d, J = 8.2 Hz, 1H), 2.71 (m, 4H), 1.75 (m, 4H) |
| 378 | H NMR (400 MHz, DMSO-d6) δ 12.98 (d, J = 6.6 Hz, 1H), 12.39 (s, 1H), |
| | 8.86 (d, J = 6.7 Hz, 1H), 8.33 (dd, J = 8.1, 1.2 Hz, 1H), 7.83 (t, J = 8.3 Hz, |
| | 1H), 7.77 (d, J = 7.7 Hz, 1H), 7.69 (s, 1H), 7.55 (t, J = 8.1 Hz, 1H), 7.31 |
| | (dd, J = 8.8, 2.4 Hz, 1H), 7.06 (d, J = 8.8 Hz, 1H), 3.85 (s, 3H) |
| 379 | 1H NMR (300 MHz, DMSO-d6) δ 12.79 (s, 1H), 10.30 (s, 1H), 8.85 (s, 1H), |
| | 8.32 (d, J = 7.8 Hz, 1H), 8.06 (s, 1H), 7.93 (s, 1H), 7.81 (t, J = 7.8 Hz, 1H), |
| | 7.74 (d, J = 6.9 Hz, 1H), 7.73 (s, 1H), 7.53 (t, J = 6.9 Hz, 1H), 2.09 (s, 3H). |
| 381 | H NMR (400 MHz, DMSO-d6) δ 12.78 (br s, 1H), 11.82 (s, 1H), 10.86 |
| | (s, 1H), 8.83 (s, 1H), 8.28 (dd, J = 8.1, 1.0 Hz, 1H), 7.75 (t, J = 8.3 Hz, 1H), |
| | 7.69 (d, J = 7.7 Hz, 1H),, 7.49-7.43 (m, 3H), 7.23 (m, 1H), 6.32 (m, 1H), |
| | 1.39 (s, 9H) |
| 382 | 1H NMR (CD3OD, 300 MHz) δ 8.83 (s, 1H), 8.40 (d, J = 7.4 Hz, 1H), |
| | 7.81-7.25 (m, 2H), 7.65 (d, J = 8.3 Hz, 1H), 7.51 (d, J = 8.2, 1H), 7.24 |
| | (d, J = 8.3, 1H), 2.58 (t, J = 7.7 Hz, 2H), 2.17 (s, 3H), 1.60 (m, 2H), 0.97 |
| | (t, J = 7.4 Hz, 3H). |
| 383 | H NMR (400 MHz, DMSO-d6) δ 1.27 (t, J = 7.5 Hz, 3H), 2.70 (q, J = 7.7 Hz, |
| | 2H), 7.05 (m, 2H), 7.47 (d, J = 8.4 Hz, 1H), 7.55 (t, J = 8.1 Hz, 1H), 7.76 |
| | (d, J = 7.7 Hz, 1H), 7.83 (t, J = 8.3 Hz, 1H), 8.13 (s, 1H), 8.35 (d, J = 6.9 Hz, |
| | 1H), 8.89 (d, J = 6.7 Hz, 1H), 10.73 (s, 1H), 12.46 (s, 1H), 12.91 (s, 1H) |
| 386 | H NMR (400 MHz, DMSO-d6) δ 13.18 (d, J = 6.8 Hz, 1H), 12.72 (s, 1H), |
| | 8.88 (d, J = 6.8 Hz, 1H), 8.34 (d, J = 8.1 Hz, 1H), 8.09 (s, 1H), 7.86-7.79 |
| | (m, 2H), 7.58-7.50 (m, 2H), 7.43 (d, J = 8.2 Hz, 1H), 3.51 (s, 2H), 1.36 (s, 6H) |
| 393 | 1H NMR (300 MHz, MeOH) δ 8.78 (s, 1H), 8.45 (d, J = 2.1 Hz, 1H), 8.16 |
| | (d, J = 8.1 Hz, 1H), 7.71 (t, J = 6.9, Hz, 1H), 7.56 (d, J = 8.7 Hz, 1H), 7.39 |
| | (m, 3H), 7.18 (m, 2H), 7.06 (m, 2H), 4.02 (m, 2H), 1.13 (t, J = 6.9, Hz, 3H); |
| 399 | 1H-NMR (CD3OD, 300 MHz) δ 8.91 (s, 1H), 8.51 (s, 1H), 8.42 (d, J = 8.3 Hz, |
| | 1H), 7.84 (t, J = 7.2 Hz, 1H), 7.67 (d, J = 9.0 Hz, 1H), 7.56 (t, J = 7.9 Hz, 1H), |
| | 7.46 (d, J = 8.5 Hz, 1H), 7.24 (d, J = 6.0 Hz, 1H), 3.48 (m, 1H), 3.09 (s, 3H, |
| | NMe), 1.39 (d, J = 6.8 Hz, 6H). |
| 412 | H NMR (400 MHz, DMSO-d6) δ 12.81-12.79 (m, 2H), 10.96 (s, 1H), 8.87 |
| | (d, J = 6.7 Hz, 1H), 8.35 (d, J = 8.1 Hz, 1H), 7.99 (d, J = 8.6 Hz, 1H), |
| | 7.83-7.73 (m, 3H), 7.53 (t, J = 8.1 Hz, 1H), 7.36 (m, 1H), 6.52 (m, 1H), |
| | 4.51 (q, J = 7.1 Hz, 2H), 1.37 (t, J = 7.1 Hz, 3H) |
| 415 | H NMR (400 MHz, DMSO-d6) δ 12.26 (s, 1H), 9.46 (s, 1H), 8.99 (s,1H), |
| | 8.43-8.41 (m, 1H), 7.94-7.88 (m, 2H), , 7.65-7.61 (m, 1H), 7.38 (d, J = 2.1 Hz, |
| | 1H), 7.10 (d, J = 8.4 Hz, 1H), 6.96 (dd, 1H), 4.08 (s, 3H), 1.35 (s, 9H) |
| 420 | H NMR (400 MHz, DMSO-d6) δ 12.91 (bs, 1H), 12.51 (s, 1H), 8.89 (s, 1H), |
| | 8.33 (dd, J = 8, 1 Hz, 2H), 7.82 (ddd, J = 8, 8, 1 Hz, 1H), 7.75 (dd, J = 8, 1 Hz, |
| | 1H), 7.70 (d, J = 9 Hz, 2H), 7.54 (ddd, J = 8, 8, 1 Hz, 1H), 4.09 (q, J = 7 Hz, |
| | 2H), 1.51 (s, 6H), 1.13 (t, J = 7 Hz, 3H). |
| 423 | H NMR (400 MHz, DMSO-d6) δ 12.91 (br s, 1H), 12.48 (s, 1H), 10.81 |
| | (d, J = 1.8 Hz, 1H), 8.89 (s, 1H), 8.35 (dd, J = 8.2, 1.1 Hz, 1H), 8.14 (d, J = 1.6 |
| | Hz, 1H), 7.82 (t, J = 7.6 Hz, 1H), 7.76 (d, J = 7.8 Hz, 1H), 7.56-7.48 (m, 2H), |
| | 7.11 (d, J = 2.2 Hz, 1H), 7.05 (dd, J = 8.5, 1.8 Hz, 1H), 3.62 (t, J = 7.3 Hz, 2H), |
| | 3.48 (q, J = 7.0 Hz, 2H), 2.91 (t, J = 7.3 Hz, 2H), 1.14 (t, J = 7.0 Hz, 3H) |
| 425 | 1H-NMR (DMSO d6, 300 MHz) δ 8.84 (s, 1H), 8.29 (d, J = 8.1 Hz, 1H), |
| | 7.78-7.70 (m, 2H), 7.61 (d, J = 8.4 Hz, 2H), 7.50 (t, J = 7.8 Hz, 1H), 7.20 |
| | (d, J = 8.7 Hz, 2H), 2.85 (h, J = 6.9 Hz, 1H), 1.19 (d, J = 6.9 Hz, 6H). |
| 427 | H NMR (400 MHz, DMSO-d6) δ 1.45 (s, 9H), 2.84 (t, J = 5.9 Hz, 2H), |
| | 3.69 (m, 2H), 4.54 (s, 1H), 6.94 (d, J = 7.5 Hz, 1H), 7.22 (t, J = 7.9 Hz, 1H), |
| | 7.55 (m, 1H), 7.77 (d, J = 7.7 Hz, 1H), 7.83 (m, 1H), 8.24 (d, J = 8.0 Hz, |
| | 1H), 8.37 (d, J = 9.2 Hz, 1H), 8.91 (s, 1H), 12.36 (s, 1H), 12.99 (s, 1H) |
| 428 | 1H NMR (300 MHz, CD3OD) δ 12.30 (s, 1H), 8.83 (s, 1H), 8.38 (d, J = 7.4 Hz, |
| | 1H), 7.78 (app dt, J = 1.1, 7.1 Hz, 1H), 7.64 (d, J= 8..3 Hz, 1H), 7.53 |
| | (app t, J = 7.5 Hz, 1H), 7.21 (br d, J = 0.9 Hz, 1H), 7.15 (d, J = 8.4 Hz, 1H), |
| | 6.98 (dd, J = 2.1, 8.4 Hz, 1H), 1.38 (s, 9H) |
| 429 | H NMR (400 MHz, DMSO-d6) δ 13.13 (d, J = 6.8 Hz, 1H), 12.63 (s, 1H), |
| | 8.86 (d, J = 6.8 Hz, 1H), 8.33 (d, J = 7.0 Hz, 1H), 7.84 (t, J = 8.3 Hz, 1H), |
| | 7.78 (d, J = 7.6 Hz, 1H), 7.56 (t, J = 8.1 Hz, 1H), 7.51 (s, 1H), 7.30 (s, 1H), |
| | 6.77 (s, 1H) |
| 433 | H NMR (400 MHz, DMSO-d6) δ 12.87 (br s, 1H), 11.82 (s, 1H), 9.20 (s, 1H), |
| | 8.87 (s, 1H), 8.33 (dd, J = 8.2, 1.1 Hz, 1H), 7.81 (t, J = 8.3 Hz, 1H), 7.75 |
| | (d, J = 7.7 Hz, 1H), 7.52 (t, J = 8.1 Hz, 1H), 7.17 (s, 1H), 7.10 (s, 1H), |
| | 1.38 (s, 9H), 1.36 (s, 9H) |
| 438 | H NMR (400 MHz, DMSO-d6) δ 12.97 (d, J = 6.6 Hz, 1H), 12.08 (s, 1H), |
| | 8.90 (d, J = 6.8 Hz, 1H), 8.35-8.34 (m, 1H), 8.03 (s, 1H), 7.85-7.81 (m, 1H), |
| | 7.77-7.71 (m, 1H), 7.58-7.44 (m, 2H), 1.46 (s, 9H), 1.42 (s, 9H) |
| 441 | 1H-NMR (d6-Acetone, 300 MHz) δ 11.90 (br s, 1H), 8.93 (br s, 1H), 8.42 |
| | (d, J = 8.1 Hz, 1H), 8.08 (s, 1H), 7.92 (s, 1H), 7.79 (m, 2H), 7.57 (m, 1H), |
| | 7.36 (s, 1H), 3.13 (s, 3H). |
| 444 | H NMR (400 MHz, DMSO-d6) δ 12.56 (s, 1H), 12.17 (br d, J = 6 Hz, 1H), |
| | 8.89 (d, J = 6 Hz, 1H), 8.42 (dd, J = 9, 2 Hz, 1H), 7.77 (d, J = 2 Hz, 1H), |
| | 7.68 (dd, J = 9, 2 Hz, 1H), 7.60 (ddd, J = 9, 9, 2 Hz, 1H), 7.46-7.40 (m, 3H), |
| | 3.47 (s, 3H), 1.35 (s, 9H). |
| 448 | H NMR (400 MHz, DMSO-d6) δ 12.96 (br s, 1H), 12.42 (s, 1H), 8.88 (s, 1H), |
| | 8.33 (dd, J = 8.2, 1.1 Hz, 1H), 7.82 (t, J = 8.3 Hz, 1H), 7.75 (d, J = 7.7 Hz, 1H), |
| | 7.66 (d, J = 8.7 Hz, 2H), 7.54 (t, J = 8.1 Hz, 1H), 7.39 (d, J = 8.7 Hz, 2H), |
| | 1.29 (s, 9H) |
| 453 | H NMR (400 MHz, DMSO-d6) δ 12.95 (d, J = 6.5 Hz, 1H), 12.38 (s, 1H), |
| | 8.86 (d, J = 6.8 Hz, 1H), 8.33 (d, J = 8.1 Hz, 1H), 7.83 (t, J = 8.3 Hz, 1H), |
| | 7.76 (d, J = 7.8 Hz, 1H), 7.54 (t, J = 8.1 Hz, 1H), 7.28 (d, J = 2.4 Hz, 1H), |
| | 7.15 (d, J = 8.6 Hz, 1H), 6.94 (dd, J = 8.6, 2.4 Hz, 1H) |
| 458 | H NMR (400 MHz, DMSO-d6) δ 12.97 (d, J = 7.1 Hz, 1H), 12.39 (s, 1H), |
| | 8.88 (d, J = 6.8 Hz, 1H), 8.33 (d, J = 7.9 Hz, 1H), 7.83 (t, J = 7.6 Hz, 1H), |
| | 7.75 (d, J = 8.2 Hz, 1H), 7.55 (t, J = 7.6 Hz, 1H), 7.47 (s, 1H), 7.17 (s, 2H), |
| | 4.04 (t, J = 5.0 Hz, 2H), 3.82 (t, J = 5.0 Hz, 2H), 1.36 (s, 9H) |
| 461 | 1H-NMR (d6-DMSO, 300 MHz) δ 11.97 (s, 1H), 8.7 (s, 1H), 8.30 (d, J = 7.7 Hz, |
| | 1H), 8.07 (d, J = 7.7 Hz, 1H), 7.726-7.699 (m, 2H), 7.446-7.357 (m, 6H), |
| | 7.236-7.178 (m, 2H). 13C-NMR (d6-DMSO, 75 MHz) d 176.3, 163.7, 144.6, |
| | 139.6, 138.9, 136.3, 134.0, 133.4, 131.0, 129.8, 129.2, 128.4, 128.1, 126.4, |
| | 126.0, 125.6, 124.7, 123.6, 119.6, 111.2. |
| 463 | 1H-NMR (DMSO d6, 300 MHz) δ 8.83 (s, 1H), 8.29 (d, J = 7.8 Hz, 1H), |
| | 7.78-7.70 (m, 2H), 7.61 (d, J = 7.8 Hz, 2H), 7.51 (t, 1H), 7.17 (d, J = 8.1 Hz, |
| | 2H), 2.57 (q, J = 7.5 Hz, 2H), 1.17 (t, J = 7.5 Hz,1H), 0.92 (t, J = 7.8 Hz, 3H). |
| 464 | H NMR (400 MHz, DMSO-d6) δ 1.37 (s, 9H), 1.38 (s, 9H), 6.80 (dd, J = 8.1, |
| | 0.9 Hz, 1H), 7.15 (m, 3H), 7.66 (t, J = 8.2 Hz, 1H), 8.87 (d, J = 6.9 Hz, 1H), |
| | 9.24 (s, 1H), 11.07 (s, 1H), 13.23 (d, J = 6.5 Hz, 1H), 13.65 (s, 1H) |
| 465 | H NMR (400 MHz, DMSO-d6) δ 12.94 (d, J = 6.0 Hz, 1H), 12.40 (s, 1H), |
| | 8.87 (d, J = 6.8 Hz, 1H), 8.33 (d, J = 8.2 Hz, 1H), 7.84-7.75 (m, 3H), 7.57-7.43 |
| | (m, 2H), 7.31 (d, J = 8.6 Hz, 1H), 4.40 (d, J = 5.8 Hz, 2H), 1.44 (s, 9H), 1.38 |
| | (s, 9H) |
| 471 | 1H-NMR (CD3OD, 300 MHz) δ 8.87 (s, 1H), 8.44 (d, J = 8.25, 1H), 8.18 |
| | (m, 1H), 7.79 (t, J = 6.88, 1H), 7.67 (d, J = 8.25, 1H), 7.54 (t, J = 7.15, 1H), |
| | 7.23 (d, J = 6.05, 1H), 7.16 (d, J = 8.5, 1H), 3.73 (s, 3H), 2.75 (t, J = 6.87, 2H), |
| | 1.7 (q, 2H), 1.03 (t, J = 7.42, 3H) |
| 476 | H NMR (400 MHz, DMSO-d6) δ 13.00 (d, J = 6.4 Hz, 1H), 12.91 (s, 1H), |
| | 10.72 (s, 1H), 8.89 (d, J = 6.8 Hz, 1H), 8.34 (d, J = 8.2 Hz, 1H), 8.16 (s, 1H), |
| | 7.85-7.75 (m, 2H), 7.56-7.54 (m, 1H), 7.44 (s, 1H), 1.35 (s, 9H) |
| 478 | H NMR (400 MHz, DMSO-d6) δ 1.40 (s, 9H), 6.98 (d, J = 2.4 Hz, 1H), 7.04 |
| | (dd, J = 8.6, 1.9 Hz, 1H), 7.55 (t, J = 8.1 Hz, 1H), 7.66 (d, J = 8.6 Hz, 1H), |
| | 7.76 (d, J = 7.7 Hz, 1H), 7.83 (t, J = 8.3 Hz, 1H), 8.13 (d, J = 1.7 Hz, 1H), |
| | 8.35 (d, J = 8.1 Hz, 1H), 8.89 (d, J = 6.7 Hz, 1H), 10.74 (s, 1H), 12.44 (s, 1H), |
| | 12.91 (s, 1H) |
| 484 | 1H NMR (300 MHz, DMSO-d6) δ 12.90 (d, J = 6.3 Hz, 1H), 12.21 (s, 1H), |
| | 8.85 (d, J = 6.8 Hz, 1H), 8.31 (d, J = 8.0 Hz, 1H), 7.79 (app dt, J = 12, 8.0 Hz, |
| | 1H), 7.72 (d, J = 8.3 Hz, 1H), 7.52 (dd, J = 6.9, 8.1 Hz, 1H), 7.05 (d, J = 8.3 Hz, |
| | 1H), 6.94 (s with fine str, 1H), 1H), 6.90 (d with fine str, J = 8.4 Hz, 1H), |
| | 2.81 (s, 3H), 1.34 (s, 9H) |
| 485 | 1H NMR (300 MHz, CDCl₃) δ 13.13 (br s, 1H), 12.78 (s, 1H), 8.91 (br s, 1H), |
| | 8.42 (br s, 1H), 8.37 (d, J = 8.1 Hz, 1H), 7.72-7.58 (m, 2H), 7.47-7.31(m, 3H), |
| | 3.34 (s, 6H), 1.46 (s, 9H) |

B) Assays for Detecting and Measuring ΔF508-CFTR Correction Properties of Compounds

I) Membrane Potential Optical Methods for Assaying ΔF508-CFTR Modulation Properties of Compounds

The optical membrane potential assay utilized voltage-sensitive FRET sensors described by Gonzalez and Tsien (See, Gonzalez, J. E. and R. Y. Tsien (1995) “Voltage sensing by fluorescence resonance energy transfer in single cells” Biophys J 69(4): 1272-80, and Gonzalez, J. E. and R. Y. Tsien (1997) “Improved indicators of cell membrane potential that use fluorescence resonance energy transfer” Chem Biol 4(4): 269-77) in combination with instrumentation for measuring fluorescence changes such as the Voltage/Ion Probe Reader (VIPR) (See, Gonzalez, J. E., K. Oades, et al. (1999) “Cell-based assays and instrumentation for screening ion-channel targets” Drug Discov Today 4(9): 431-439).

These voltage sensitive assays are based on the change in fluorescence resonant energy transfer (FRET) between the membrane-soluble, voltage-sensitive dye, DiSBAC₂(3), and a fluorescent phospholipid, CC2-DMPE, which is attached to the outer leaflet of the plasma membrane and acts as a FRET donor. Changes in membrane potential (Vₘ) cause the negatively charged DiSBAC₂(3) to redistribute across the plasma membrane and the amount of energy transfer from CC2-DMPE changes accordingly. The changes in fluorescence emission were monitored using VIPR™ II, which is an integrated liquid handler and fluorescent detector designed to conduct cell-based screens in 96- or 384-well microtiter plates.

Identification of Correction Compounds

To identify small molecules that correct the trafficking defect associated with ΔF508-CFTR; a single-addition HTS assay format was developed. The cells were incubated in serum-free medium for 16 hrs at 37° C. in the presence or absence (negative control) of test compound. As a positive control, cells plated in 384-well plates were incubated for 16 hrs at 27° C. to “temperature-correct” ΔF508-CFTR. The cells were subsequently rinsed 3× with Krebs Ringers solution and loaded with the voltage-sensitive dyes. To activate ΔF508-CFTR, 10 μM forskolin and the CFTR potentiator, genistein (20 μM), were added along with Cl⁻-free medium to each well. The addition of Cl⁻-free medium promoted Cl⁻ efflux in response to ΔF508-CFTR activation and the resulting membrane depolarization was optically monitored using the FRET-based voltage-sensor dyes.

Identification of Potentiator Compounds

To identify potentiators of ΔF508-CFTR, a double-addition HTS assay format was developed. During the first addition, a Cl⁻-free medium with or without test compound was added to each well. After 22 sec, a second addition of Cl⁻-free medium containing 2-10 μM forskolin was added to activate ΔF508-CFTR. The extracellular Cl⁻ concentration following both additions was 28 mM, which promoted Cl⁻ efflux in response to ΔF508-CFTR activation and the resulting membrane depolarization was optically monitored using the FRET-based voltage-sensor dyes. Solutions

Bath Solution #1: (in mM) NaCl 160, KCl 4.5, CaCl₂ 2, MgCl₂ 1, HEPES 10, pH 7.4 with NaOH.
Chloride-free bath solution: Chloride salts in Bath Solution #1 are substituted with gluconate salts.
CC2-DMPE: Prepared as a 10 mM stock solution in DMSO and stored at −20° C.
DiSBAC₂(3): Prepared as a 10 mM stock in DMSO and stored at −20° C.

Cell Culture

NIH3T3 mouse fibroblasts stably expressing ΔF508-CFTR are used for optical measurements of membrane potential. The cells are maintained at 37° C. in 5% CO₂ and 90% humidity in Dulbecco's modified Eagle's medium supplemented with 2 mM glutamine, 10% fetal bovine serum, 1× NEAA, β-ME, 1× pen/strep, and 25 mM HEPES in 175 cm² culture flasks. For all optical assays, the cells were seeded at 30,000/well in 384-well matrigel-coated plates and cultured for 2 hrs at 37° C. before culturing at 27° C. for 24 hrs. for the potentiator assay. For the correction assays, the cells are cultured at 27° C. or 37° C. with and without compounds for 16-24 hours B) Electrophysiological Assays for assaying ΔF508-CFTR modulation properties of compounds

1. Ussing Chamber Assay

Ussing chamber experiments were performed on polarized epithelial cells expressing ΔF508-CFTR to further characterize the ΔF508-CFTR modulators identified in the optical assays. FRT^{ΔF508-CFTR} epithelial cells grown on Costar Snapwell cell culture inserts were mounted in an Using chamber (Physiologic Instruments, Inc., San Diego, Calif.), and the monolayers were continuously short-circuited using a Voltage-clamp System (Department of Bioengineering, University of Iowa, IA, and, Physiologic Instruments, Inc., San Diego, Calif.). Transepithelial resistance was measured by applying a 2-mV pulse. Under these conditions, the FRT epithelia demonstrated resistances of 4 KΩ/cm² or more. The solutions were maintained at 27° C. and bubbled with air. The electrode offset potential and fluid resistance were corrected using a cell-free insert. Under these conditions, the current reflects the flow of Cl⁻ through ΔF508-CFTR expressed in the apical membrane. The I_{SC} was digitally acquired using an MP100A-CE interface and AcqKnowledge software (v3.2.6; BIOPAC Systems, Santa Barbara, Calif.).

Identification of Correction Compounds

Typical protocol utilized a basolateral to apical membrane Cl⁻ concentration gradient. To set up this gradient, normal ringer was used on the basolateral membrane, whereas apical NaCl was replaced by equimolar sodium gluconate (titrated to pH 7.4 with NaOH) to give a large Cl⁻ concentration gradient across the epithelium. All experiments were performed with intact monolayers. To fully activate ΔF508-CFTR, forskolin (10 μM) and the PDE inhibitor, IBMX (100 μM), were applied followed by the addition of the CFTR potentiator, genistein (50 μM).

As observed in other cell types, incubation at low temperatures of FRT cells stably expressing ΔF508-CFTR increases the functional density of CFTR in the plasma membrane. To determine the activity of correction compounds, the cells were incubated with 10 μM of the test compound for 24 hours at 37° C. and were subsequently washed 3× prior to recording. The cAMP- and genistein-mediated I_{SC} in compound-treated cells was normalized to the 27° C. and 37° C. controls and expressed as percentage activity. Preincubation of the cells with the correction compound significantly increased the cAMP- and genistein-mediated I_{SC} compared to the 37° C. controls.

Identification of Potentiator Compounds

Typical protocol utilized a basolateral to apical membrane Cl⁻ concentration gradient. To set up this gradient, normal ringers was used on the basolateral membrane and was permeabilized with nystatin (360 μg/ml), whereas apical NaCl was replaced by equimolar sodium gluconate (titrated to pH 7.4 with NaOH) to give a large Cl⁻ concentration gradient across the epithelium. All experiments were performed 30 min after nystatin permeabilization. Forskolin (10 μM) and all test compounds were added to both sides of the cell culture inserts. The efficacy of the putative ΔF508-CFTR potentiators was compared to that of the known potentiator, genistein.

Solutions
Basolateral solution (in mM): NaCl (135), CaCl₂(1.2), MgCl₂ (1.2), K₂HPO₄(2.4), KHPO₄ (0.6), N-2-hydroxyethylpiperazine-N′-2-ethanesulfonic acid (HEPES) (10), and dextrose (10). The solution was titrated to pH 7.4 with NaOH.
Apical solution (in mM): Same as basolateral solution with NaCl replaced with Na Gluconate (135).

Cell Culture

Fisher rat epithelial (FRT) cells expressing ΔF508-CFTR (FRT^{ΔF508-CFTR}) were used for Using chamber experiments for the putative ΔF508-CFTR modulators identified from our optical assays. The cells were cultured on Costar Snapwell cell culture inserts and cultured for five days at 37° C. and 5% CO₂ in Coon's modified Ham's F-12 medium supplemented with 5% fetal calf serum, 100 U/ml penicillin, and 100 μg/ml streptomycin. Prior to use for characterizing the potentiator activity of compounds, the cells were incubated at 27° C. for 16-48 hrs to correct for the ΔF508-CFTR. To determine the activity of corrections compounds, the cells were incubated at 27° C. or 37° C. with and without the compounds for 24 hours.

2. Whole-Cell Recordings

The macroscopic ΔF508-CFTR current (I_{ΔF508}) in temperature- and test compound-corrected NIH3T3 cells stably expressing ΔF508-CFTR were monitored using the perforated-patch, whole-cell recording. Briefly, voltage-clamp recordings of I_{ΔF508} were performed at room temperature using an Axopatch 200B patch-clamp amplifier (Axon Instruments Inc., Foster City, Calif.). All recordings were acquired at a sampling frequency of 10 kHz and low-pass filtered at 1 kHz. Pipettes had a resistance of 5-6 MΩ when filled with the intracellular solution. Under these recording conditions, the calculated reversal potential for Cl⁻ (E_{Cl}) at room temperature was −28 mV. All recordings had a seal resistance >20 GΩ and a series resistance <15 MΩ. Pulse generation, data acquisition, and analysis were performed using a PC equipped with a Digidata 1320 A/D interface in conjunction with Clampex 8 (Axon Instruments Inc.). The bath contained <250 μl of saline and was continuously perfused at a rate of 2 ml/min using a gravity-driven perfusion system.

Identification of Correction Compounds

To determine the activity of correction compounds for increasing the density of functional ΔF508-CFTR in the plasma membrane, we used the above-described perforated-patch-recording techniques to measure the current density following 24-hr treatment with the correction compounds. To fully activate ΔF508-CFTR, 10 μM forskolin and 20 μM genistein were added to the cells. Under our recording conditions, the current density following 24-hr incubation at 27° C. was higher than that observed following 24-hr incubation at 37° C. These results are consistent with the known effects of low-temperature incubation on the density of Δ508-CFTR in the plasma membrane. To determine the effects of correction compounds on CFTR current density, the cells were incubated with 10 μM of the test compound for 24 hours at 37° C. and the current density was compared to the 27° C. and 37° C. controls (% activity). Prior to recording, the cells were washed 3× with extracellular recording medium to remove any remaining test compound. Preincubation with 10 μM of correction compounds significantly increased the cAMP- and genistein-dependent current compared to the 37° C. controls.

Identification of Potentiator Compounds

The ability of ΔF508-CFTR potentiators to increase the macroscopic ΔF508-CFTR Cl⁻ current (I_{ΔF508}) in NIH3T3 cells stably expressing ΔF508-CFTR was also investigated using perforated-patch-recording techniques. The potentiators identified from the optical assays evoked a dose-dependent increase in I_{ΔF508} with similar potency and efficacy observed in the optical assays. In all cells examined, the reversal potential before and during potentiator application was around −30 mV, which is the calculated E_{Cl} (−28 mV).

Solutions
Intracellular solution (in mM): Cs-aspartate (90), CsCl (50), MgCl₂ (1), HEPES (10), and 240 μg/ml amphotericin-B (pH adjusted to 7.35 with CsOH).
Extracellular solution (in mM): N-methyl-D-glucamine (NMDG)-Cl (150), MgCl₂ (2), CaCl₂ (2), HEPES (10) (pH adjusted to 7.35 with HCl).

Cell Culture

NIH3T3 mouse fibroblasts stably expressing ΔF508-CFTR are used for whole-cell recordings. The cells are maintained at 37° C. in 5% CO₂ and 90% humidity in Dulbecco's modified Eagle's medium supplemented with 2 mM glutamine, 10% fetal bovine serum, 1×NEAA, β-ME, 1× pen/strep, and 25 mM HEPES in 175 cm² culture flasks. For whole-cell recordings, 2,500-5,000 cells were seeded on poly-L-lysine-coated glass coverslips and cultured for 24-48 hrs at 27° C. before use to test the activity of potentiators; and incubated with or without the correction compound at 37° C. for measuring the activity of correctors.

3. Single-Channel Recordings

The single-channel activities of temperature-corrected ΔF508-CFTR stably expressed in NIH3T3 cells and activities of potentiator compounds were observed using excised inside-out membrane patch. Briefly, voltage-clamp recordings of single-channel activity were performed at room temperature with an Axopatch 200B patch-clamp amplifier (Axon Instruments Inc.). All recordings were acquired at a sampling frequency of 10 kHz and low-pass filtered at 400 Hz. Patch pipettes were fabricated from Corning Kovar Sealing #7052 glass (World Precision Instruments, Inc., Sarasota, Fla.) and had a resistance of 5-8 MΩ when filled with the extracellular solution. The ΔF508-CFTR was activated after excision, by adding 1 mM Mg-ATP, and 75 nM of the cAMP-dependent protein kinase, catalytic subunit (PKA; Promega Corp. Madison, Wis.). After channel activity stabilized, the patch was perfused using a gravity-driven microperfusion system. The inflow was placed adjacent to the patch, resulting in complete solution exchange within 1-2 sec. To maintain ΔF508-CFTR activity during the rapid perfusion, the nonspecific phosphatase inhibitor F⁻ (10 mM NaF) was added to the bath solution. Under these recording conditions, channel activity remained constant throughout the duration of the patch recording (up to 60 min). Currents produced by positive charge moving from the intra- to extracellular solutions (anions moving in the opposite direction) are shown as positive currents. The pipette potential (Vₚ) was maintained at 80 mV.

Channel activity was analyzed from membrane patches containing ≦2 active channels. The maximum number of simultaneous openings determined the number of active channels during the course of an experiment. To determine the single-channel current amplitude, the data recorded from 120 sec of ΔF508-CFTR activity was filtered “off-line” at 100 Hz and then used to construct all-point amplitude histograms that were fitted with multigaussian functions using Bio-Patch Analysis software (Bio-Logic Comp. France). The total microscopic current and open probability (Pₒ) were determined from 120 sec of channel activity. The Pₒ was determined using the Bio-Patch software or from the relationship Pₒ=I/i(N), where I=mean current, i=single-channel current amplitude, and N=number of active channels in patch.

Solutions
Extracellular solution (in mM): NMDG (150), aspartic acid (150), CaCl₂ (5), MgCl₂ (2), and HEPES (10) (pH adjusted to 7.35 with Tris base).
Intracellular solution (in mM): NMDG-Cl (150), MgCl₂ (2), EGTA (5), TES (10), and Tris base (14) (pH adjusted to 7.35 with HCl).

Cell Culture

NIH3T3 mouse fibroblasts stably expressing ΔF508-CFTR are used for excised-membrane patch-clamp recordings. The cells are maintained at 37° C. in 5% CO₂ and 90% humidity in Dulbecco's modified Eagle's medium supplemented with 2 mM glutamine, 10% fetal bovine serum, 1×NEAA, β-ME, 1× pen/strep, and 25 mM HEPES in 175 cm² culture flasks. For single channel recordings, 2,500-5,000 cells were seeded on poly-L-lysine-coated glass coverslips and cultured for 24-48 hrs at 27° C. before use.

Compounds of the invention are useful as modulators of ATP binding cassette transporters. Table II.A-4 below illustrates the EC50 and relative efficacy of certain embodiments in Table I.

In Table II.A-4 below, the following meanings apply:

EC50: “+++” means <10 uM; “++” means between 10 uM to 25 uM; “+” means between 25 uM to 60 uM. % Efficacy: “+” means <25%; “++” means between 25% to 100%, “+++” means >100%.

TABLE II.A-4

| | | EC50 |
|---|---|---|
| Cmpd # | (um) | % Activity |
| 1 | +++ | ++ |
| 2 | +++ | ++ |
| 3 | +++ | ++ |
| 4 | +++ | ++ |
| 5 | ++ | ++ |
| 6 | +++ | +++ |
| 7 | + | + |
| 8 | +++ | ++ |
| 9 | + | + |
| 10 | +++ | ++ |
| 11 | +++ | ++ |
| 12 | +++ | ++ |
| 13 | +++ | ++ |
| 14 | +++ | ++ |
| 15 | ++ | ++ |
| 16 | +++ | ++ |
| 17 | +++ | ++ |
| 18 | +++ | ++ |
| 19 | ++ | + |
| 20 | +++ | ++ |
| 21 | + | + |
| 22 | ++ | ++ |
| 23 | +++ | ++ |
| 24 | + | + |
| 25 | ++ | ++ |
| 26 | +++ | ++ |
| 28 | ++ | ++ |
| 29 | ++ | ++ |
| 30 | +++ | ++ |
| 31 | +++ | ++ |
| 32 | +++ | ++ |
| 33 | +++ | ++ |
| 34 | +++ | ++ |
| 35 | +++ | ++ |
| 36 | +++ | ++ |
| 37 | +++ | ++ |
| 38 | +++ | ++ |
| 39 | ++ | ++ |
| 40 | + | + |
| 41 | +++ | ++ |
| 42 | +++ | ++ |
| 43 | +++ | ++ |
| 44 | ++ | ++ |
| 46 | ++ | ++ |
| 47 | +++ | ++ |
| 48 | +++ | ++ |
| 49 | +++ | ++ |
| 50 | +++ | ++ |
| 51 | +++ | ++ |
| 52 | +++ | ++ |
| 53 | + | + |
| 54 | + | + |
| 55 | + | + |
| 56 | +++ | ++ |
| 57 | ++ | +++ |
| 58 | +++ | ++ |
| 59 | +++ | +++ |
| 60 | +++ | ++ |
| 61 | +++ | ++ |
| 62 | +++ | ++ |
| 63 | +++ | ++ |
| 64 | + | + |
| 65 | +++ | ++ |
| 66 | ++ | ++ |
| 67 | +++ | ++ |
| 68 | +++ | ++ |
| 69 | +++ | ++ |
| 70 | ++ | ++ |
| 71 | +++ | ++ |
| 72 | +++ | ++ |
| 73 | + | + |
| 74 | + | + |
| 75 | + | + |
| 76 | +++ | ++ |
| 77 | +++ | ++ |
| 78 | + | + |
| 79 | +++ | ++ |
| 80 | +++ | ++ |
| 81 | + | + |
| 82 | +++ | ++ |
| 83 | +++ | ++ |
| 84 | + | + |
| 85 | +++ | ++ |
| 86 | ++ | ++ |
| 87 | +++ | ++ |
| 88 | +++ | ++ |
| 89 | + | + |
| 90 | +++ | ++ |
| 91 | +++ | ++ |
| 92 | +++ | ++ |
| 93 | +++ | ++ |
| 94 | +++ | ++ |
| 95 | ++ | ++ |
| 96 | +++ | ++ |
| 97 | +++ | ++ |
| 98 | +++ | ++ |
| 99 | +++ | ++ |
| 100 | + | + |
| 101 | +++ | ++ |
| 102 | ++ | ++ |
| 103 | +++ | +++ |
| 104 | +++ | ++ |
| 105 | ++ | ++ |
| 106 | + | + |
| 107 | ++ | ++ |
| 108 | +++ | ++ |
| 109 | ++ | ++ |
| 110 | + | + |
| 111 | +++ | ++ |
| 112 | +++ | ++ |
| 113 | +++ | ++ |
| 114 | +++ | ++ |
| 115 | +++ | ++ |
| 116 | +++ | ++ |
| 117 | +++ | ++ |
| 118 | +++ | ++ |
| 119 | +++ | ++ |
| 120 | ++ | ++ |
| 122 | + | + |
| 123 | +++ | ++ |
| 124 | +++ | +++ |
| 125 | ++ | ++ |
| 126 | +++ | ++ |
| 127 | +++ | ++ |
| 128 | + | + |
| 129 | ++ | ++ |
| 130 | +++ | ++ |
| 131 | +++ | ++ |
| 132 | + | + |
| 133 | ++ | ++ |
| 134 | +++ | ++ |
| 135 | +++ | +++ |
| 136 | +++ | ++ |
| 137 | +++ | ++ |
| 138 | +++ | ++ |
| 139 | +++ | ++ |
| 140 | +++ | ++ |
| 141 | ++ | ++ |
| 142 | +++ | ++ |
| 143 | +++ | ++ |
| 144 | +++ | ++ |
| 145 | +++ | ++ |
| 146 | + | + |
| 147 | +++ | ++ |
| 148 | +++ | ++ |
| 149 | ++ | ++ |
| 150 | +++ | ++ |
| 151 | +++ | ++ |
| 152 | + | + |
| 153 | +++ | ++ |
| 154 | + | + |
| 155 | + | + |
| 156 | +++ | ++ |
| 157 | +++ | ++ |
| 158 | +++ | ++ |
| 159 | ++ | ++ |
| 160 | +++ | ++ |
| 161 | +++ | ++ |
| 162 | + | + |
| 163 | ++ | ++ |
| 164 | +++ | ++ |
| 165 | + | + |
| 166 | +++ | ++ |
| 167 | ++ | ++ |
| 168 | + | + |
| 169 | ++ | ++ |
| 170 | + | + |
| 171 | +++ | ++ |
| 172 | +++ | ++ |
| 173 | + | + |
| 174 | +++ | ++ |
| 175 | ++ | ++ |
| 176 | +++ | ++ |
| 177 | +++ | +++ |
| 178 | +++ | ++ |
| 179 | + | + |
| 180 | +++ | ++ |
| 181 | +++ | ++ |
| 182 | +++ | ++ |
| 183 | +++ | ++ |
| 184 | + | + |
| 185 | + | + |
| 186 | +++ | ++ |
| 187 | +++ | ++ |
| 188 | +++ | ++ |
| 189 | +++ | ++ |
| 190 | +++ | ++ |
| 191 | + | + |
| 192 | + | + |
| 193 | ++ | ++ |
| 194 | + | + |
| 195 | + | + |
| 196 | +++ | ++ |
| 197 | + | + |
| 198 | +++ | ++ |
| 199 | +++ | ++ |
| 200 | ++ | ++ |
| 201 | ++ | + |
| 202 | +++ | ++ |
| 203 | +++ | ++ |
| 204 | +++ | ++ |
| 205 | +++ | ++ |
| 206 | +++ | ++ |
| 207 | +++ | ++ |
| 208 | +++ | ++ |
| 209 | ++ | ++ |
| 210 | ++ | ++ |
| 211 | +++ | ++ |
| 212 | + | + |
| 213 | +++ | ++ |
| 214 | ++ | ++ |
| 215 | +++ | ++ |
| 216 | + | + |
| 217 | ++ | ++ |
| 218 | +++ | ++ |
| 219 | + | + |
| 220 | +++ | ++ |
| 221 | +++ | ++ |
| 222 | ++ | ++ |
| 223 | +++ | ++ |
| 224 | +++ | ++ |
| 225 | +++ | ++ |
| 226 | +++ | ++ |
| 227 | + | + |
| 228 | +++ | ++ |
| 229 | +++ | ++ |
| 230 | ++ | ++ |
| 231 | +++ | ++ |
| 232 | ++ | ++ |
| 233 | ++ | + |
| 234 | +++ | ++ |
| 235 | +++ | ++ |
| 236 | +++ | ++ |
| 237 | +++ | ++ |
| 238 | +++ | ++ |
| 239 | +++ | ++ |
| 240 | +++ | ++ |
| 241 | ++ | ++ |
| 242 | +++ | ++ |
| 243 | ++ | ++ |
| 244 | +++ | ++ |
| 245 | +++ | ++ |
| 246 | +++ | ++ |
| 247 | +++ | ++ |
| 248 | ++ | ++ |
| 249 | ++ | ++ |
| 250 | + | + |
| 251 | +++ | ++ |
| 252 | ++ | ++ |
| 253 | +++ | ++ |
| 254 | +++ | ++ |
| 255 | +++ | ++ |
| 256 | + | + |
| 257 | +++ | ++ |
| 258 | +++ | ++ |
| 259 | +++ | ++ |
| 260 | +++ | ++ |
| 261 | +++ | ++ |
| 262 | +++ | ++ |
| 263 | +++ | ++ |
| 264 | ++ | ++ |
| 265 | +++ | ++ |
| 266 | +++ | ++ |
| 267 | +++ | ++ |
| 268 | ++ | ++ |
| 269 | +++ | ++ |
| 270 | +++ | ++ |
| 271 | +++ | ++ |
| 272 | ++ | ++ |
| 273 | +++ | +++ |
| 274 | +++ | ++ |
| 275 | ++ | ++ |
| 276 | ++ | ++ |
| 277 | +++ | +++ |
| 278 | +++ | ++ |
| 279 | +++ | ++ |
| 280 | + | + |
| 281 | +++ | ++ |
| 282 | +++ | ++ |
| 283 | +++ | +++ |
| 284 | ++ | ++ |
| 285 | +++ | ++ |
| 286 | +++ | +++ |
| 287 | +++ | ++ |
| 288 | +++ | ++ |
| 289 | +++ | ++ |
| 290 | +++ | ++ |
| 291 | +++ | ++ |
| 292 | +++ | ++ |
| 293 | ++ | +++ |
| 294 | ++ | ++ |
| 295 | +++ | ++ |
| 296 | ++ | ++ |
| 297 | +++ | ++ |
| 298 | +++ | ++ |
| 299 | +++ | ++ |
| 300 | +++ | ++ |
| 301 | + | + |
| 302 | ++ | ++ |
| 303 | ++ | ++ |
| 304 | +++ | ++ |
| 305 | +++ | +++ |
| 306 | +++ | +++ |
| 307 | +++ | ++ |
| 308 | ++ | ++ |
| 309 | + | + |
| 310 | +++ | ++ |
| 311 | +++ | ++ |
| 312 | +++ | ++ |
| 313 | +++ | ++ |
| 314 | +++ | ++ |
| 315 | +++ | ++ |
| 316 | ++ | ++ |
| 317 | +++ | ++ |
| 318 | ++ | ++ |
| 319 | +++ | ++ |
| 320 | +++ | ++ |
| 321 | +++ | ++ |
| 322 | +++ | ++ |
| 323 | +++ | ++ |
| 324 | +++ | ++ |
| 325 | +++ | ++ |
| 326 | ++ | ++ |
| 327 | +++ | ++ |
| 328 | + | + |
| 329 | ++ | ++ |
| 330 | +++ | ++ |
| 331 | + | + |
| 332 | +++ | ++ |
| 333 | +++ | ++ |
| 334 | ++ | ++ |
| 335 | + | + |
| 336 | +++ | ++ |
| 337 | +++ | ++ |
| 338 | ++ | ++ |
| 339 | +++ | ++ |
| 340 | +++ | ++ |
| 341 | +++ | ++ |
| 342 | +++ | ++ |
| 343 | ++ | ++ |
| 344 | +++ | ++ |
| 345 | +++ | ++ |
| 346 | +++ | ++ |
| 347 | ++ | ++ |
| 348 | +++ | ++ |
| 349 | +++ | ++ |
| 350 | +++ | ++ |
| 351 | +++ | ++ |
| 352 | +++ | ++ |
| 353 | +++ | ++ |
| 354 | +++ | ++ |
| 355 | +++ | ++ |
| 356 | +++ | ++ |
| 357 | +++ | ++ |
| 358 | +++ | ++ |
| 359 | ++ | ++ |
| 360 | +++ | ++ |
| 361 | +++ | +++ |
| 362 | +++ | ++ |
| 363 | +++ | +++ |
| 364 | +++ | ++ |
| 365 | ++ | ++ |
| 366 | +++ | ++ |
| 367 | +++ | ++ |
| 368 | +++ | ++ |
| 369 | ++ | + |
| 370 | +++ | ++ |
| 371 | +++ | ++ |
| 372 | +++ | ++ |
| 373 | +++ | ++ |
| 374 | + | + |
| 375 | +++ | ++ |
| 376 | + | + |
| 377 | ++ | ++ |
| 378 | ++ | ++ |
| 379 | ++ | ++ |
| 380 | +++ | ++ |
| 381 | +++ | ++ |
| 382 | +++ | ++ |
| 383 | +++ | ++ |
| 384 | +++ | ++ |
| 385 | +++ | ++ |
| 386 | +++ | ++ |
| 387 | +++ | ++ |
| 388 | +++ | ++ |
| 389 | +++ | ++ |
| 390 | + | + |
| 391 | +++ | ++ |
| 392 | + | + |
| 393 | +++ | ++ |
| 394 | + | + |
| 395 | +++ | ++ |
| 396 | ++ | ++ |
| 397 | +++ | ++ |
| 398 | ++ | ++ |
| 399 | +++ | ++ |
| 400 | + | + |
| 401 | +++ | ++ |
| 402 | +++ | + |
| 403 | +++ | ++ |
| 404 | +++ | ++ |
| 405 | +++ | ++ |
| 406 | +++ | ++ |
| 407 | +++ | ++ |
| 408 | +++ | ++ |
| 409 | +++ | ++ |
| 410 | +++ | +++ |
| 411 | +++ | ++ |
| 412 | +++ | ++ |
| 413 | ++ | ++ |
| 414 | + | + |
| 415 | +++ | ++ |
| 416 | +++ | ++ |
| 417 | +++ | ++ |
| 418 | ++ | ++ |
| 419 | + | + |
| 420 | +++ | ++ |
| 421 | +++ | ++ |
| 423 | +++ | ++ |
| 424 | +++ | ++ |
| 425 | +++ | ++ |
| 426 | +++ | ++ |
| 427 | +++ | ++ |
| 428 | +++ | ++ |
| 429 | +++ | ++ |
| 430 | +++ | ++ |
| 431 | +++ | ++ |
| 432 | +++ | ++ |
| 433 | +++ | ++ |
| 434 | +++ | ++ |
| 435 | +++ | ++ |
| 436 | +++ | ++ |
| 437 | + | + |
| 438 | +++ | ++ |
| 439 | +++ | ++ |
| 440 | +++ | ++ |
| 441 | +++ | ++ |
| 442 | + | + |
| 443 | + | + |
| 444 | +++ | ++ |
| 445 | +++ | +++ |
| 446 | + | + |
| 447 | ++ | ++ |
| 448 | +++ | ++ |
| 449 | +++ | ++ |
| 450 | ++ | ++ |
| 451 | +++ | ++ |
| 452 | +++ | ++ |
| 453 | +++ | ++ |
| 454 | + | + |
| 455 | +++ | ++ |
| 456 | +++ | ++ |
| 457 | +++ | ++ |
| 458 | +++ | ++ |
| 459 | +++ | ++ |
| 460 | +++ | ++ |
| 461 | +++ | ++ |
| 462 | +++ | ++ |
| 463 | +++ | ++ |
| 464 | +++ | ++ |
| 465 | +++ | ++ |
| 466 | +++ | ++ |
| 467 | + | + |
| 468 | + | + |
| 469 | +++ | ++ |
| 470 | +++ | ++ |
| 471 | +++ | ++ |
| 472 | +++ | ++ |
| 473 | ++ | ++ |
| 474 | + | + |
| 476 | +++ | ++ |
| 477 | + | + |
| 478 | +++ | ++ |
| 479 | +++ | ++ |
| 480 | + | + |
| 481 | +++ | ++ |
| 482 | ++ | ++ |
| 483 | +++ | ++ |
| 484 | +++ | ++ |
| 485 | +++ | ++ |

### II.A.2 Embodiments of Formula A1

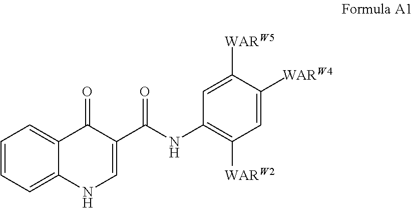

or pharmaceutically acceptable salts thereof, wherein:

Each of WR^{W2} and WR^{W4} is independently selected from CN, CF₃, halo, C₂₋₆ straight or branched alkyl, C₃₋₁₂ membered cycloaliphatic, phenyl, a 5-10 membered heteroaryl or 3-7 membered heterocyclic, wherein said heteroaryl or heterocyclic has up to 3 heteroatoms selected from O, S, or N, wherein said WR^{W2} and WR^{W4} is independently and optionally substituted with up to three substituents selected from —OR′, —CF₃, —OCF₃, SR′, S(O)R′, SO₂R′, —SCF₃, halo, CN, —COOR′, —COR′, —O(CH₂)₂N(R′)₂, —O(CH₂)N(R′)₂, —CON(R′)₂, —(CH₂)₂OR′, —(CH₂)OR′, —CH₂CN, optionally substituted phenyl or phenoxy, —N(R′)₂, —NR′C(O)OR′, —NR′C(O)R′, —(CH₂)₂N(R′)₂, or —(CH₂)N(R′)₂; WR^{W5} is selected from hydrogen, —OCF₃, —CF₃, —OH, —OCH₃, —NH₂, —CN, —CHF₂, —NHR′, —N(R′)₂, —NHC(O)R′, —NHC(O)OR′, —NHSO₂R′, —CH₂OH, —CH₂N(R′)₂, —C(O)OR′, —SO₂NHR′, —SO₂N(R′)₂, or —CH₂NHC(O)OR′; and

Each R′ is independently selected from an optionally substituted group selected from a C₁₋₈ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or two occurrences of R′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur;

provided that:

i) WR^{W2} and WR^{W4} are not both —Cl; and

WR^{W2}, WR^{W4} and WR^{W5} are not —OCH₂CH₂Ph, —OCH₂CH₂(2-trifluoromethyl-phenyl), —OCH₂CH₂-(6,7-dimethoxy-1,2,3,4-tetrahydroisoquinolin-2-yl), or substituted 1H-pyrazol-3-yl;

### Compound of Formula A1

In one embodiment of the compound of Formula A1, each of WAR^{W2} and WAR^{W4} is independently selected from CN, CF₃, halo, C₂₋₆ straight or branched alkyl, C₃₋₁₂ membered cycloaliphatic, or phenyl, wherein said WAR^{W2} and WAR^{W4} is independently and optionally substituted with up to three substituents selected from —OR′, —CF₃, —OCF₃, —SCF₃, halo, —COOAR′, —COAR′, —O(CH₂)₂N(AR′)₂, —O(CH₂)N(AR′)₂, —CON(AR′)₂, —(CH₂)₂OAR′, —(CH₂)OAR′, optionally substituted phenyl, —N(AR′)₂, —NC(O)OAR′, —NC(O)AR′, —(CH₂)₂N(AR′)₂, or —(CH₂)N(AR′)₂; and WAR^{W5} is selected from hydrogen, —OCF₃, —CF₃, —OH, —OCH₃, —NH₂, —CN, —NHAR′, —N(AR′)₂, —NHC(O)AR′, —NHC(O)OAR′, —NHSO₂AR′, —CH₂OH, —C(O)OAR′, —SO₂NHAR′, or —CH₂NHC(O)O-AR′).

Alternatively, each of WAR^{W2} and WAR^{W4} is independently selected from —CN, —CF₃, C₂₋₆ straight or branched alkyl, C₃₋₁₂ membered cycloaliphatic, or phenyl, wherein each of said WAR^{W2} and WAR^{W4} is independently and optionally substituted with up to three substituents selected from —OAR′, —CF₃, —OCF₃, —SCF₃, halo, —COOAR′, —COAR′, —O(CH₂)₂N(AR′)₂, —O(CH₂)N(AR′)₂, —CON(AR′)₂, —(CH₂)₂OAR′, —(CH₂)OAR′, optionally substituted phenyl, —N(AR′)₂, —NC(O)OAR′, —NC(O)AR′, —(CH₂)₂N(AR′)₂, or —(CH₂)N(AR′)₂; and WAR^{W5} is selected from —OH, —CN, —NHR′, —N(AR′)₂, —NHC(O)AR′, —NHC(O)OAR′, —NHSO₂AR′, —CH₂OH, —C(O)OAR′, —SO₂NHAR′, or —CH₂NHC(O)O-(AR′).

In a further embodiment, WAR^{W2} is a phenyl ring optionally substituted with up to three substituents selected from —OR′, —CF₃, —OCF₃, —SAR′, —S(O)AR′, —SO₂AR′, —SCF₃, halo, —CN, —COOAR′, —COAR′, —O(CH₂)₂N(AR′)₂, —O(CH₂)N(AR′)₂, —CON(AR′)₂, —(CH₂)₂OAR′, —(CH₂)OAR′, —CH₂CN, optionally substituted phenyl or phenoxy, —N(AR′)₂, —NAR′C(O)OAR′, —NAR′C(O)AR′, —(CH₂)₂N(AR′)₂, or —(CH₂)N(AR′)₂; WAR^{W4} is C₂₋₆ straight or branched alkyl; and WAR^{W5} is —OH.

In another embodiment, each of WAR^{W2} and WAR^{W4} is independently —CF₃, —CN, or a C₂₋₆ straight or branched alkyl.

In another embodiment, each of WAR^{W2} and WAR^{W4} is C₂₋₆ straight or branched alkyl optionally substituted with up to three substituents independently selected from —OR′, —CF₃, —OCF₃, —SAR′, —S(O)AR′, —SO₂AR′, —SCF₃, halo, —CN, —COOAR′, —COAR′, —O(CH₂)₂N(AR′)₂, —O(CH₂)N(AR′)₂, —CON(AR′)₂, —(CH₂)₂OAR′, —(CH₂)OAR′, —CH₂CN, optionally substituted phenyl or phenoxy, —N(AR′)₂, —NAR′C(O)OAR′, —NAR′C(O)AR′, —(CH₂)₂N(AR′)₂, or —(CH₂)N(AR′)₂.

In another embodiment, each of WAR^{W2} and WAR^{W4} is independently selected from optionally substituted n-propyl, isopropyl, n-butyl, sec-butyl, t-butyl, 1,1-dimethyl-2-hydroxyethyl, 1,1-dimethyl-2-(ethoxycarbonyl)-ethyl, 1,1-dimethyl-3-(t-butoxycarbonyl-amino)propyl, or n-pentyl.

In another embodiment, WAR^{W5} is selected from —CN, —NHR′, —N(AR′)₂, —CH₂N(AR′)₂, —NHC(O)AR′, —NHC(O)OAR′, —OH, C(O)OAR′, or —SO₂NHAR′.

In another embodiment, WAR^{W5} is selected from —CN, —NH(C₁₋₆ alkyl), —N(C₁₋₆ alkyl)₂, —NHC(O)(C₁₋₆ alkyl), —CH₂NHC(O)O(C₁₋₆ alkyl), —NHC(O)O(C₁₋₆ alkyl), —OH, —O(C₁₋₆ alkyl), —C(O)O(C₁₋₆ alkyl), —CH₂O(C₁₋₆ alkyl), or —SO₂NH₂.

In another embodiment, WAR^{W5} is selected from —OH, —CH₂OH, —NHC(O)OMe, —NHC(O)OEt, —CN, —CH₂NHC(O)O(t-butyl), —C(O)OMe, or —SO₂NH₂.

In another embodiment:

WAR^{W2} is C₂₋₆ straight or branched alkyl;

WAR^{W4} is C₂₋₆ straight or branched alkyl or monocyclic or bicyclic aliphatic; and

WAR^{W5} is selected from —CN, —NH(C₁₋₆ alkyl), —N(C₁₋₆alkyl)₂, —NHC(O)(C₁₋₅ alkyl), —NHC(O)O(C₁₋₆ alkyl), —CH₂C(O)O(C₁₋₆ alkyl), —OH, —O(C₁₋₆ alkyl), —C(O)O(C₁₋₆ alkyl), or —SO₂NH₂.

In another embodiment:

WAR^{W2} is C₂₋₄ alkyl, —CF₃, —CN, or phenyl optionally substituted with up to 3 substituents selected from C₁₋₄ alkyl, —O(C₁₋₄ alkyl), or halo;

WAR^{W4} is —CF₃, C₂₋₆ alkyl, or C₆₋₁₀ cycloaliphatic; and

WAR^{W5} is —OH, —NH(C₁₋₆ alkyl), or —N(C₁₋₆alkyl)₂.

In another embodiment, WAR^{W2} is tert-butyl.

In another embodiment, WAR^{W4} is tert-butyl.

In another embodiment, WAR^{W5} is —OH.

### II.A.3. Compound 1

In another embodiment, the compound of Formula A1 is Compound 1.

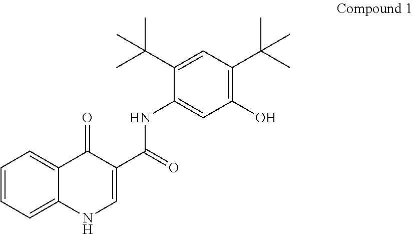 [SMILES: CC(C)(C)c1cc(C(C)(C)C)c(NC(=O)c2c[nH]c3ccccc3c2=O)cc1O]

Compound 1 is known by the name N-[2,4-bis(1,1-dimethylethyl)-5-hydroxyphenyl]-1,4-dihydro-4-oxoquinoline-3-carboxamide and by the name N-(5-hydroxy-2,4-di-tert-butyl-phenyl)-4-oxo-1H-quinoline-3-carboxamide.

Synthesis of the compounds of formula A1

Compounds of Formula A1

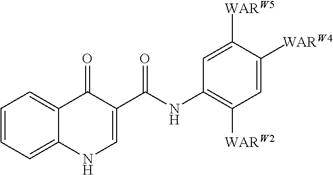

are readily prepared by combining an acid moiety

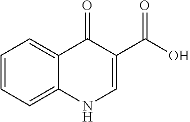 [SMILES: O=C(O)c1c[nH]c2ccccc2c1=O]

with an amine moiety

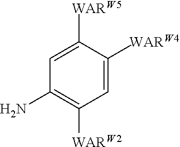

as described herein, wherein WAR^{W2}, WAR^{W4}, and WAR^{W5} are as defined previously.

a. Synthesis of the Acid Moiety of Compounds of Formula A1

The acid precursor of compounds of Formula A1, dihydroquinoline carboxylic acid, can be synthesized according to Scheme 1-1, by conjugate addition of EtOCH═C(COOEt)₂ to aniline, followed by thermal rearrangement and hydrolysis.

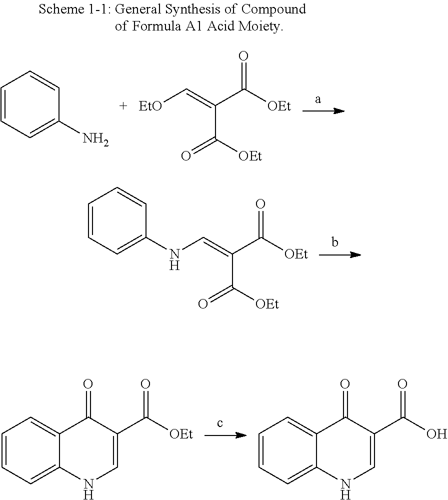

a) 140-150° C.; b) PPA, POCl₃, 70° C. or diphenyl ether, 220° C.; c) i) 2N NaOH ii) 2N HCl

b. Synthesis of the Amine Moiety of Compounds of formula A1

Amine precursors of compounds of Formula A1 are prepared as depicted in Scheme 1-2, wherein WAR^{W2}, WAR^{W4}, and WAR^{W5} are as defined previously. Thus, ortho alkylation of the para-substituted benzene in step (a) provides a tri-substituted intermediate. Optional protection when WAR^{W5} is OH (step (b) and nitration (step c) provides the trisubstituted nitrated intermediate. Optional deprotection (step d) and hydrogenation (step e) provides the desired amine moiety.

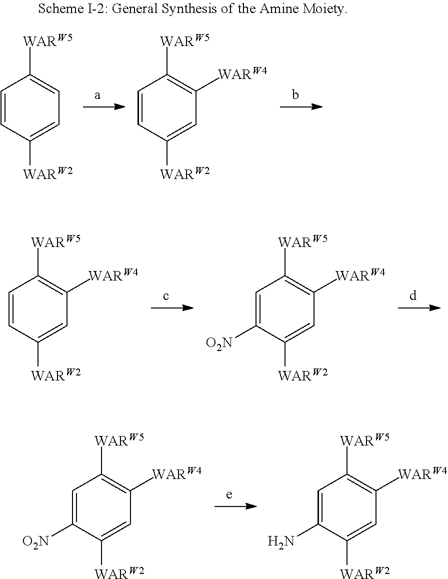

a) WAR^{W4}OH, WAR^{W4}=alkyl; b) ClCO₂R, TEA; c) HNO₃, H₂SO₄; d) base; e) hydrogenation.

c. Coupling of Acid Moiety to Amine Moiety to Form Compounds of formula A1

Compounds of Formula A1 are prepared by coupling an acid moiety with an amine moiety as depicted in Scheme 1-3. In general, the coupling reaction requires a coupling reagent, a base, as well as a solvent. Examples of conditions used include HATU, DIEA; BOP, DIEA, DMF; HBTU, Et₃N, CH₂Cl₂; PFPTFA, pyridine.

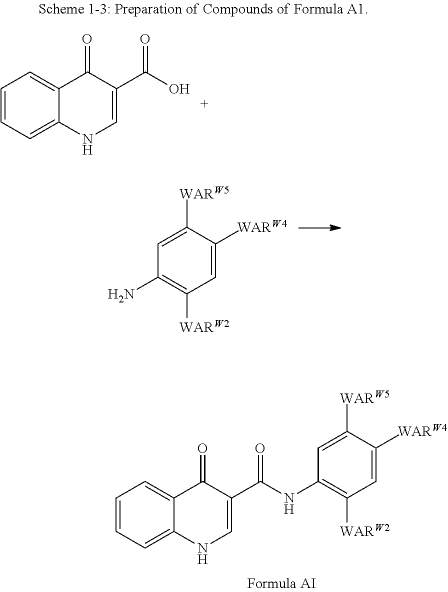

### 2. Compound 1 Synthesis

Compound 1 can be prepared generally as provided in Schemes 1-3 through 1-6, wherein an acid moiety

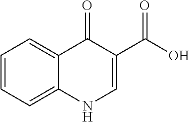 [SMILES: O=C(O)c1c[nH]c2ccccc2c1=O]

is coupled with an amine moiety

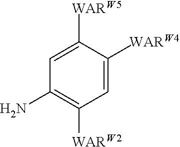

wherein WAR^{W2} and WAR^{W4} are t-butyl, and WAR^{W5} is OH. More detailed schemes and examples are provided below.

a. Synthesis of Compound 1 Acid Moiety

The synthesis of the acid moiety 4-Oxo-1,4-dihydroquinoline-3-carboxylic acid 26, is summarized in Scheme 1-4.

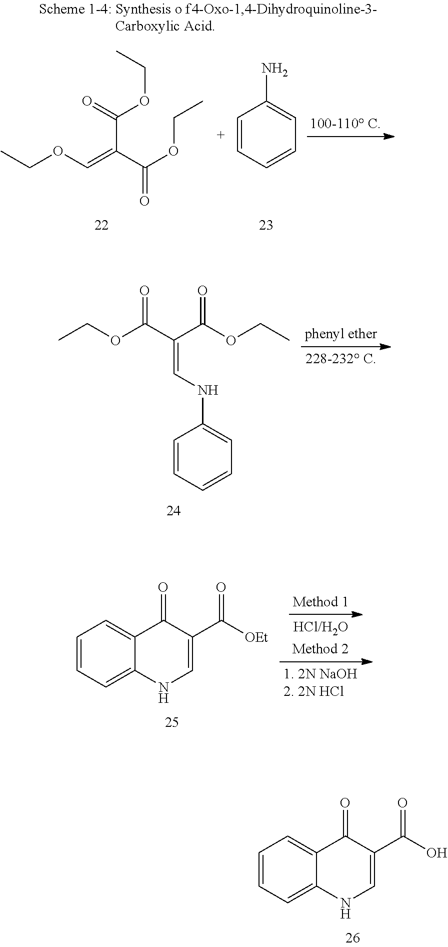

### Ethyl 4-oxo-1,4-dihydroquinoline-3-carboxylate (25)

Compound 23 (4.77 g, 47.7 mmol) was added dropwise to Compound 22 (10 g, 46.3 mmol) with subsurface N₂ flow to drive out ethanol below 30° C. for 0.5 hours. The solution was then heated to 100-110° C. and stirred for 2.5 hours. After cooling the mixture to below 60° C., diphenyl ether was added. The resulting solution was added dropwise to diphenyl ether that had been heated to 228-232° C. for 1.5 hours with subsurface N₂ flow to drive out ethanol. The mixture was stirred at 228-232° C. for another 2 hours, cooled to below 100° C. and then heptane was added to precipitate the product. The resulting slurry was stirred at 30° C. for 0.5 hours. The solids were then filtrated, and the cake was washed with heptane and dried in vacuo to give Compound 25 as a brown solid. ¹H NMR (DMSO-d₆; 400 MHz) δ 12.25 (s), δ 8.49 (d), δ 8.10 (m), δ 7.64 (m), δ 7.55 (m), δ 7.34 (m), δ 4.16 (q), δ 1.23 (t).

### -Oxo-1,4-dihydroquinoline-3-carboxylic acid (26)

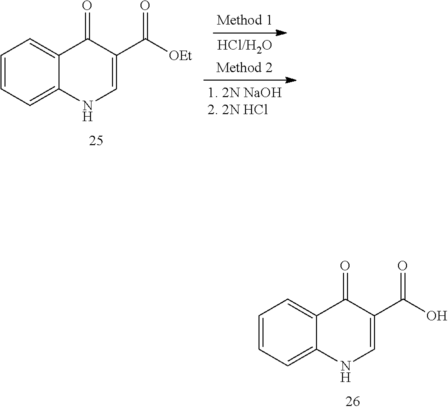 [SMILES: CCOC(=O)c1c[nH]c2ccccc2c1=O ; O=C(O)c1c[nH]c2ccccc2c1=O]

### Method 1

Compound 25 (1.0 eq) was suspended in a solution of HCl (10.0 eq) and H₂O (11.6 vol). The slurry was heated to 85-90° C., although alternative temperatures are also suitable for this hydrolysis step. For example, the hydrolysis can alternatively be performed at a temperature of from about 75 to about 100° C. In some instances, the hydrolysis is performed at a temperature of from about 80 to about 95° C. In others, the hydrolysis step is performed at a temperature of from about 82 to about 93° C. (e.g., from about 82.5 to about 92.5° C. or from about 86 to about 89° C.). After stirring at 85-90° C. for approximately 6.5 hours, the reaction was sampled for reaction completion. Stirring may be performed under any of the temperatures suited for the hydrolysis. The solution was then cooled to 20-25° C. and filtered. The reactor/cake was rinsed with H₂O (2 vol×2). The cake was then washed with 2 vol H₂O until the pH ≧3.0. The cake was then dried under vacuum at 60° C. to give Compound 26.

### Method 2

Compound 25 (11.3 g, 52 mmol) was added to a mixture of 10% NaOH (aq) (10 mL) and ethanol (100 mL). The solution was heated to reflux for 16 hours, cooled to 20-25° C. and then the pH was adjusted to 2-3 with 8% HCl. The mixture was then stirred for 0.5 hours and filtered. The cake was washed with water (50 mL) and then dried in vacuo to give Compound 26 as a brown solid. ¹H NMR (DMSO-d₆; 400 MHz) δ 15.33 (s), δ 13.39 (s), δ 8.87 (s), δ 8.26 (m), δ 7.87 (m), δ 7.80 (m), δ 7.56 (m).

b. Synthesis of Compound 1 Amine Moiety

The synthesis of the amine moiety 32, is summarized in Scheme 1-5.

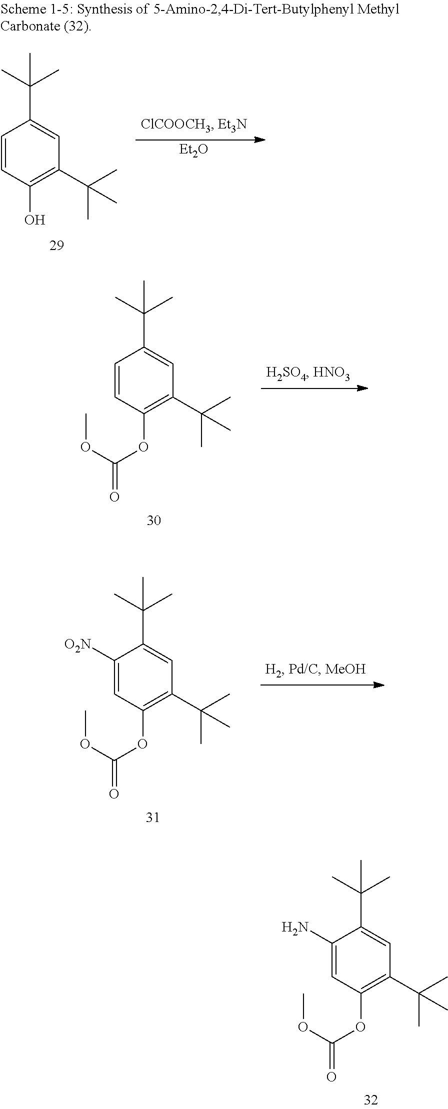 [SMILES: "Et2O" ; CC(C)(C)c1ccc(O)c(C(C)(C)C)c1 ; COC(=O)Oc1ccc(C(C)(C)C)cc1C(C)(C)C ; COC(=O)Oc1cc([N+](=O)[O-])c(C(C)(C)C)cc1C(C)(C)C ; COC(=O)Oc1cc(N)c(C(C)(C)C)cc1C(C)(C)C]

### 2,4-Di-tert-butylphenyl methyl carbonate (30)

### Method 1

To a solution of 2,4-di-tert-butyl phenol, 29, (10 g, 48.5 mmol) in diethyl ether (100 mL) and triethylamine (10.1 mL, 72.8 mmol), was added methyl chloroformate (7.46 mL, 97 mmol) dropwise at 0° C. The mixture was then allowed to warm to room temperature and stir for an additional 2 hours. An additional 5 mL triethylamine and 3.7 mL methyl chloroformate was then added and the reaction stirred overnight. The reaction was then filtered, the filtrate was cooled to 0° C., and an additional 5 mL triethylamine and 3.7 mL methyl chloroformate was then added and the reaction was allowed to warm to room temperature and then stir for an addition 1 hours. At this stage, the reaction was almost complete and was worked up by filtering, then washing with water (2×), followed by brine. The solution was then concentrated to produce a yellow oil and purified using column chromatography to give Compound 30. ¹H NMR (400 MHz, DMSO-d₆) δ 7.35 (d, J=2.4 Hz, 1H), 7.29 (dd, J=8.4, 2.4 Hz, 1H), 7.06 (d, J=8.4 Hz, 1H), 3.85 (s, 3H), 1.30 (s, 9H), 1.29 (s, 9H).

### Method 2

To a reactor vessel charged with 4-dimethylaminopyridine (DMAP, 3.16 g, 25.7 mmol) and 2,4-ditert-butyl phenol (Compound 29, 103.5 g, 501.6 mmol) was added methylene chloride (415 g, 313 mL) and the solution was agitated until all solids dissolved. Triethylamine (76 g, 751 mmol) was then added and the solution was cooled to 0-5° C. Methyl chloroformate (52 g, 550.3 mmol) was then added dropwise over 2.5-4 hours, while keeping the solution temperature between 0-5° C. The reaction mixture was then slowly heated to 23-28° C. and stirred for 20 hours. The reaction was then cooled to 10-15° C. and charged with 150 mL water. The mixture was stirred at 15-20° C. for 35-45 minutes and the aqueous layer was then separated and extracted with 150 mL methylene chloride. The organic layers were combined and neutralized with 2.5% HCl (aq) at a temperature of 5-20° C. to give a final pH of 5-6. The organic layer was then washed with water and concentrated in vacuo at a temperature below 20° C. to 150 mL to give Compound 30 in methylene chloride.

### 5-Nitro-2,4-di-tert-butylphenyl methyl carbonate (31)

### Method 1

To a stirred solution of Compound 30 (6.77 g, 25.6 mmol) was added 6 mL of a 1:1 mixture of sulfuric acid and nitric acid at 0° C. dropwise. The mixture was allowed to warm to room temperature and stirred for 1 hour. The product was purified using liquid chromatography (ISCO, 120 g, 0-7% EtOAc/Hexanes, 38 min) producing about an 8:1-10:1 mixture of regioisomers of Compound 31 as a white solid. ¹H NMR (400 MHz, DMSO-d₆) δ 7.63 (s, 1H), 7.56 (s, 1H), 3.87 (s, 3H), 1.36 (s, 9H), 1.32 (s, 9H). HPLC ret. time 3.92 min 10-99% CH₃CN, 5 min run; ESI-MS 310 m/z (MH)⁺.

### Method 2

To Compound 30 (100 g, 378 mmol) was added DCM (540 g, 408 mL). The mixture was stirred until all solids dissolved, and then cooled to −5-0° C. Concentrated sulfuric acid (163 g) was then added dropwise, while maintaining the initial temperature of the reaction, and the mixture was stirred for 4.5 hours. Nitric acid (62 g) was then added dropwise over 2-4 hours while maintaining the initial temperature of the reaction, and was then stirred at this temperature for an additional 4.5 hours. The reaction mixture was then slowly added to cold water, maintaining a temperature below 5° C. The quenched reaction was then heated to 25° C. and the aqueous layer was removed and extracted with methylene chloride. The combined organic layers were washed with water, dried using Na₂SO₄, and concentrated to 124-155 mL. Hexane (48 g) was added and the resulting mixture was again concentrated to 124-155 mL. More hexane (160 g) was subsequently added to the mixture. The mixture was then stirred at 23-27° C. for 15.5 hours, and was then filtered. To the filter cake was added hexane (115 g), the resulting mixture was heated to reflux and stirred for 2-2.5 hours. The mixture was then cooled to 3-7° C., stirred for an additional 1-1.5 hours, and filtered to give Compound 31 as a pale yellow solid.

### 5-Amino-2,4-di-tert-butylphenyl methyl carbonate (32)

2,4-Di-tert-butyl-5-nitrophenyl methyl carbonate (1.00 eq) was charged to a suitable hydrogenation reactor, followed by 5% Pd/C (2.50 wt % dry basis, Johnson-Matthey Type 37). MeOH (15.0 vol) was charged to the reactor, and the system was closed. The system was purged with N₂ (g), and was then pressurized to 2.0 Bar with H₂ (g). The reaction was performed at a reaction temperature of 25° C.+/−5° C. When complete, the reaction was filtered, and the reactor/cake was washed with MeOH (4.00 vol). The resulting filtrate was distilled under vacuum at no more than 50° C. to 8.00 vol. Water (2.00 vol) was added at 45° C.+/−5° C. The resultant slurry was cooled to 0° C.+/−5. The slurry was held at 0° C.+/−5° C. for no less than 1 hour, and filtered. The cake was washed once with 0° C.+/−5° C. MeOH/H₂O (8:2) (2.00 vol). The cake was dried under vacuum (−0.90 bar and −0.86 bar) at 35° C.-40° C. to give Compound 32. ¹H NMR (400 MHz, DMSO-d₆) δ 7.05 (s, 1H), 6.39 (s, 1H), 4.80 (s, 2H), 3.82 (s, 3H), 1.33 (s, 9H), 1.23 (s, 9H).

Once the reaction was complete, the resulting mixture was diluted with from about 5 to 10 volumes of MeOH (e.g., from about 6 to about 9 volumes of MeOH, from about 7 to about 8.5 volumes of MeOH, from about 7.5 to about 8 volumes of MeOH, or about 7.7 volumes of MeOH), heated to a temperature of about 35±5° C., filtered, washed, and dried, as described above.

c. Coupling of Acid and Amine Moiety to Form Compound 1

The coupling of the acid moiety to the amine moiety is summarized in Scheme 1-6.

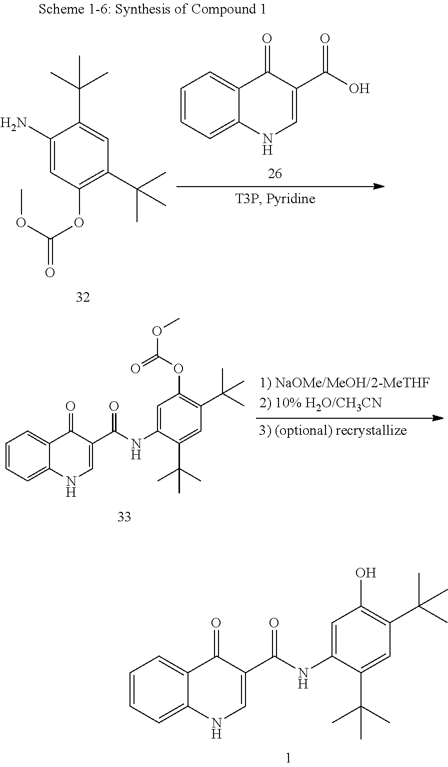 [SMILES: COC(=O)Oc1cc(N)c(C(C)(C)C)cc1C(C)(C)C ; O=C(O)c1c[nH]c2ccccc2c1=O ; COC(=O)Oc1cc(NC(=O)c2c[nH]c3ccccc3c2=O)c(C(C)(C)C)cc1C(C)(C)C ; CC(C)(C)c1cc(C(C)(C)C)c(NC(=O)c2c[nH]c3ccccc3c2=O)cc1O]

### N-(2,4-di-tert-butyl-5-hydroxyphenyl)-4-oxo-1,4-dihydroquinoline-3-carboxamide (1)

4-Oxo-1,4-dihydroquinoline-3-carboxylic acid 26 (1.0 eq) and 5-amino-2,4-di-tert-butylphenyl methyl carbonate 32 (1.1 eq) were charged to a reactor. 2-MeTHF (4.0 vol, relative to the acid) was added followed by T3P® 50% solution in 2-MeTHF (1.7 eq). The T3P charged vessel was washed with 2-MeTHF (0.6 vol). Pyridine (2.0 eq) was then added, and the resulting suspension was heated to 47.5+/−5.0° C. and held at this temperature for 8 hours. A sample was taken and checked for completion by HPLC. Once complete, the resulting mixture was cooled to 25.0° C.+/−2.5° C. 2-MeTHF was added (12.5 vol) to dilute the mixture. The reaction mixture was washed with water (10.0 vol) 2 times. 2-MeTHF was added to bring the total volume of reaction to 40.0 vol (˜16.5 vol charged). To this solution was added NaOMe/MeOH (1.7 equiv) to perform the methanolysis. The reaction was stirred for no less than 1.0 hour, and checked for completion by HPLC. Once complete, the reaction was quenched with 1N HCl (10.0 vol), and washed with 0.1N HCl (10.0 vol). The organic solution was polish filtered to remove any particulates and placed in a second reactor. The filtered solution was concentrated at no more than 35° C. (jacket temperature) and no less than 8.0° C. (internal reaction temperature) under reduced pressure to 20 vol. CH₃CN was added to 40 vol and the solution concentrated at no more than 35° C. (jacket temperature) and no less than 8.0° C. (internal reaction temperature) to 20 vol. The addition of CH₃CN and concentration cycle was repeated 2 more times for a total of 3 additions of CH₃CN and 4 concentrations to 20 vol. After the final concentration to 20 vol, 16.0 vol of CH₃CN was added followed by 4.0 vol of H₂O to make a final concentration of 40 vol of 10% H₂O/CH₃CN relative to the starting acid. This slurry was heated to 78.0° C.+/−5.0° C. (reflux). The slurry was then stirred for no less than 5 hours. The slurry was cooled to 0.0° C.+/−5° C. over 5 hours, and filtered. The cake was washed with 0.0° C.+/−5.0° C. CH₃CN (5 vol) 4 times. The resulting solid (Compound 1) was dried in a vacuum oven at 50.0° C.+/−5.0° C. ¹H NMR (400 MHz, DMSO-d₆) δ 12.8 (s, 1H), 11.8 (s, 1H), 9.2 (s, 1H), 8.9 (s, 1H), 8.3 (s, 1H), 7.2 (s, 1H), 7.9 (t, 1H), 7.8 (d, 1H), 7.5 (t, 1H), 7.1 (s, 1H), 1.4 (s, 9H), 1.4 (s, 9H).

An alternative synthesis of Compound 1 is depicted in Scheme 1-7.

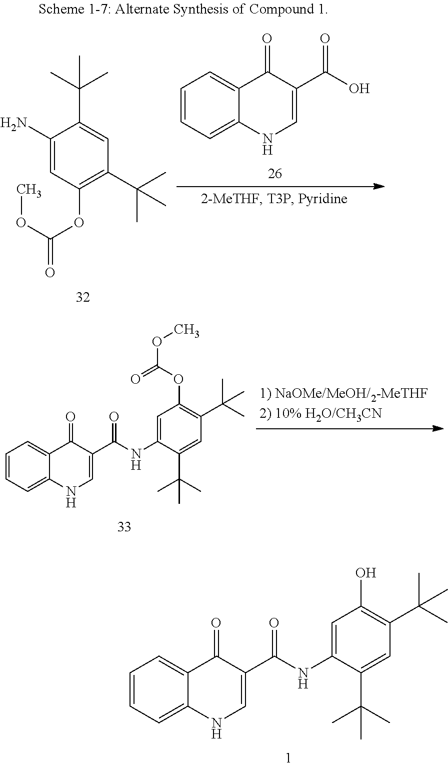 [SMILES: COC(=O)Oc1cc(N)c(C(C)(C)C)cc1C(C)(C)C ; O=C(O)c1c[nH]c2ccccc2c1=O ; COC(=O)Oc1cc(NC(=O)c2c[nH]c3ccccc3c2=O)c(C(C)(C)C)cc1C(C)(C)C ; CC(C)(C)c1cc(C(C)(C)C)c(NC(=O)c2c[nH]c3ccccc3c2=O)cc1O]

4-Oxo-1,4-dihydroquinoline-3-carboxylic acid 26 (1.0 eq) and 5-amino-2,4-di-tert-butylphenyl methyl carbonate 32 (1.1 eq) were charged to a reactor. 2-MeTHF (4.0 vol, relative to the acid) was added followed by T3P® 50% solution in 2-MeTHF (1.7 eq). The T3P charged vessel was washed with 2-MeTHF (0.6 vol). Pyridine (2.0 eq) was then added, and the resulting suspension was heated to 47.5+/−5.0° C. and held at this temperature for 8 hours. A sample was taken and checked for completion by HPLC. Once complete, the resulting mixture was cooled to 20° C.+/−5° C. 2-MeTHF was added (12.5 vol) to dilute the mixture. The reaction mixture was washed with water (10.0 vol) 2 times and 2-MeTHF (16.5 vol) was charged to the reactor. This solution was charged with 30% w/w NaOMe/MeOH (1.7 equiv) to perform the methanolysis. The reaction was stirred at 25.0° C.+/−5.0° C. for no less than 1.0 hour, and checked for completion by HPLC. Once complete, the reaction was quenched with 1.2N HCl/H₂O (10.0 vol), and washed with 0.1N HCl/H₂O (10.0 vol). The organic solution was polish filtered to remove any particulates and placed in a second reactor.

The filtered solution was concentrated at no more than 35° C. (jacket temperature) and no less than 8.0° C. (internal reaction temperature) under reduced pressure to 20 vol. CH₃CN was added to 40 vol and the solution concentrated at no more than 35° C. (jacket temperature) and no less than 8.0° C. (internal reaction temperature) to 20 vol. The addition of CH₃CN and concentration cycle was repeated 2 more times for a total of 3 additions of CH₃CN and 4 concentrations to 20 vol. After the final concentration to 20 vol, 16.0 vol of CH₃CN was charged followed by 4.0 vol of H₂O to make a final concentration of 40 vol of 10% H₂O/CH₃CN relative to the starting acid. This slurry was heated to 78.0° C.+/−5.0° C. (reflux). The slurry was then stirred for no less than 5 hours. The slurry was cooled to 20 to 25° C. over 5 hours, and filtered. The cake was washed with CH₃CN (5 vol) heated to 20 to 25° C. 4 times. The resulting solid (Compound 1) was dried in a vacuum oven at 50.0° C.+/−5.0° C. ¹H NMR (400 MHz, DMSO-d₆) δ 12.8 (s, 1H), 11.8 (s, 1H), 9.2 (s, 1H), 8.9 (s, 1H), 8.3 (s, 1H), 7.2 (s, 1H), 7.9 (t, 1H), 7.8 (d, 1H), 7.5 (t, 1H), 7.1 (s, 1H), 1.4 (s, 9H), 1.4 (s, 9H).

### II.B Embodiments of Column B Compounds

The modulators of ABC transporter activity in Column B are fully described and exemplified in Ser. No. 11/824,606, filed: Jun. 29, 2007 and commonly assigned to the Assignee of the present invention. All of the compounds recited in Ser. No. 11/824,606 are useful in the present invention and are hereby incorporated into the present disclosure in their entirety.

II.B.1 Formula B Compounds

The present invention includes a compound of Formula B:

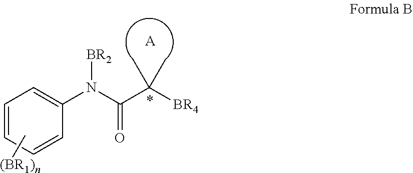

or a pharmaceutically acceptable salt thereof.

wherein each BR₁ is an optionally substituted C₁₋₆ aliphatic, an optionally substituted aryl, an optionally substituted heteroaryl, an optionally substituted C₃₋₁₀ cycloaliphatic, or an optionally substituted 4 to 10 membered heterocycloaliphatic, carboxy [e.g., hydroxycarbonyl or alkoxycarbonyl], alkoxy, amido [e.g., aminocarbonyl], amino, halo, cyano, alkylsulfanyl, or hydroxy;

provided that at least one BR₁ is an optionally substituted aryl or an optionally substituted heteroaryl and said R₁ is attached to the 3- or 4-position of the phenyl ring;

each BR₂ is hydrogen, an optionally substituted C₁₋₆ aliphatic, an optionally substituted C₃₋₆ cycloaliphatic, an optionally substituted phenyl, or an optionally substituted heteroaryl;

each BR₄ is an optionally substituted aryl or an optionally substituted heteroaryl; Each n is 1, 2, 3, 4 or 5; and

ring A is an optionally substituted cycloaliphatic or an optionally substituted heterocycloaliphatic where the atoms of ring A adjacent to C* are carbon atoms, and each of which is optionally substituted with 1, 2, or 3 substituents.

As noted in the general definitions preceding this section, all of the R variables in Column B formulas indicate that the R variable pertains to the Column B compounds. For example, BR₁ indicates that it is an R₁ variable that pertains to the Column B compounds. BR₁ is not to be mistaken as being the variable B bonded or adjacent to the variable R₁.

### Substituent BR₁

Each BR₁ is an optionally substituted C₁₋₆ aliphatic, an optionally substituted aryl, an optionally substituted heteroaryl, an optionally substituted C₃₋₁₀ cycloaliphatic, an optionally substituted 4 to 10 membered heterocycloaliphatic, carboxy [e.g., hydroxycarbonyl or alkoxycarbonyl], amido [e.g., aminocarbonyl], amino, halo, alkoxy, or hydroxy.

In some embodiments, one BR₁ is an optionally substituted C₁₋₆ aliphatic. In several examples, one BR₁ is an optionally substituted C₁₋₆ alkyl, an optionally substituted C₂₋₅ alkenyl, or an optionally substituted C₂₋₄ alkynyl. In several examples, one BR₁ is C₁₋₆ alkyl, C₂₋₄ alkenyl, or C₂₋₆ alkynyl.

In several embodiments, one BR₁ is an aryl or heteroaryl with 1, 2, or 3 substituents. In several examples, one BR₁ is a monocyclic aryl or heteroaryl. In several embodiments, BR₁ is an aryl or heteroaryl with 1, 2, or 3 substituents. In several examples, BR₁ is a monocyclic aryl or heteroaryl.

In several embodiments, at least one BR₁ is an optionally substituted aryl or an optionally substituted heteroaryl and BR₁ is bonded to the core structure at the 4-position on the phenyl ring.

In several embodiments, at least one BR₁ is an optionally substituted aryl or an optionally substituted heteroaryl and BR₁ is bonded to the core structure at the 3-position on the phenyl ring.

In several embodiments, one BR₁ is phenyl with up to 3 substituents. In several embodiments, BR₁ is phenyl with up to 2 substituents.

In several embodiments, one BR₁ is a heteroaryl ring with up to 3 substituents. In certain embodiments, one BR₁ is a monocyclic heteroaryl ring with up to 3 substituents. In other embodiments, one BR₁ is a bicyclic heteroaryl ring with up to 3 substituents. In several embodiments, BR₁ is a heteroaryl ring with up to 3 substituents.

In some embodiments, one BR₁ is an optionally substituted C₃₋₁₀ cycloaliphatic or an optionally substituted 3-8 membered heterocycloaliphatic. In several examples, one BR₁ is a monocyclic cycloaliphatic substituted with up to 3 substituents. In several examples, one BR₁ is a monocyclic heterocycloaliphatic substituted with up to 3 substituents. In one embodiment, one BR₁ is a 4 membered heterocycloaliphatic having one ring member selected from oxygen, nitrogen (including NH and NBR^{X}), or sulfur (including S, SO, and SO₂); wherein said heterocycloaliphatic is substituted with up to 3 substitutents. In one example, one BR₁ is 3-methyloxetan-3-yl.

In several embodiments, one BR₁ is carboxy [e.g., hydroxycarbonyl or alkoxycarbonyl]. Or, one BR₁ is amido [e.g., aminocarbonyl]. Or, one BR₁ is amino. Or, is halo. Or, is cyano. Or, hydroxy.

In some embodiments, BR₁ is hydrogen, methyl, ethyl, iso-propyl, tert-butyl, cyclopropyl, cyclobutyl, cyclopentyl, cyclohexyl, allyl, F, Cl, methoxy, ethoxy, iso-propoxy, tert-butoxy, CF₃, OCF₃, SCH₃, SCH₂CH₃, CN, hydroxy, or amino. In several examples, BR₁ is hydrogen, methyl, ethyl, iso-propyl, tert-butyl, methoxy, ethoxy, SCH₃, SCH₂CH₃, F, Cl, CF₃, or OCF₃. In several examples, BR₁ can be hydrogen. Or, BR₁ can be methyl. Or, BR₁ can be ethyl. Or, BR₁ can be iso-propyl. Or, BR₁ can be tert-butyl. Or, BR₁ can be F. Or, BR₁ can be Cl. Or, BR₁ can be OH. Or, BR₁ can be OCF₃. Or, BR₁ can be CF₃. Or, BR₁ can be methoxy. Or, BR₁ can be ethoxy. Or, BR₁ can be SCH₃.

In several embodiments, BR₁ is substituted with no more than three substituents independently selected from halo, oxo, or optionally substituted aliphatic, cycloaliphatic, heterocycloaliphatic, amino [e.g., (aliphatic)amino], amido [e.g., aminocarbonyl, ((aliphatic)amino)carbonyl, and ((aliphatic)₂amino)carbonyl], carboxy [e.g., alkoxycarbonyl and hydroxycarbonyl], sulfamoyl [e.g., aminosulfonyl, ((aliphatic)₂amino)sulfonyl, ((cycloaliphatic)aliphatic)aminosulfonyl, and ((cycloaliphatic)amino)sulfonyl], cyano, alkoxy, aryl, heteroaryl [e.g., monocyclic heteroaryl and bicycloheteroaryl], sulfonyl [e.g., aliphaticsulfonyl or (heterocycloaliphatic)sulfonyl], sulfinyl [e.g., aliphaticsulfinyl], aroyl, heteroaroyl, or heterocycloaliphaticcarbonyl.

In several embodiments, BR₁ is substituted with halo. Examples of BR₁ substituents include F, Cl, and Br. In several examples, BR₁ is substituted with F.

In several embodiments, BR₁ is substituted with an optionally substituted aliphatic. Examples of BR₁ substituents include optionally substituted alkoxyaliphatic, heterocycloaliphatic, aminoalkyl, hydroxyalkyl, (heterocycloalkyl)aliphatic, alkylsulfonylaliphatic, alkylsulfonylaminoaliphatic, alkylcarbonylaminoaliphatic, alkylaminoaliphatic, or alkylcarbonylaliphatic.

In several embodiments, BR₁ is substituted with an optionally substituted amino. Examples of BR₁ substituents include aliphaticcarbonylamino, aliphaticamino, arylamino, or aliphaticsulfonylamino.

In several embodiments, BR₁ is substituted with a sulfonyl. Examples of BR₁ include heterocycloaliphatic sulfonyl, aliphatic sulfonyl, aliphaticaminosulfonyl, aminosulfonyl, aliphaticcarbonylaminosulfonyl, alkoxyalkylheterocycloallcylsulfonyl, alkylheterocycloalkylsulfonyl, alkylaminosulfonyl, cycloalkylaminosulfonyl, (heterocycloalkyl)alkylaminosulfonyl, and heterocycloalkylsulfonyl.

In several embodiments, BR₁ is substituted with carboxy. Examples of BR₁ substituents include alkoxycarbonyl and hydroxycarbonyl.

In several embodiments BR₁ is substituted with amido. Examples of BR₁ substituents include alkylaminocarbonyl, aminocarbonyl, ((aliphatic)₂amino)carbonyl, and [((aliphatic)aminoaliphatic)amino]carbonyl.

In several embodiments, BR₁ is substituted with carbonyl. Examples of BR₁ substituents include arylcarbonyl, cycloaliphaticcarbonyl, heterocycloaliphaticcarbonyl, and heteroarylcarbonyl.

In several embodiments, each BR₁ is a hydroxycarbonyl, hydroxy, or halo.

In some embodiments, BR₁ is hydrogen. In some embodiments, BR₁ is —Z^{E}R₉, wherein each Z^{E} is independently a bond or an optionally substituted branched or straight C₁₋₆ aliphatic chain wherein up to two carbon units of Z^{E} are optionally and independently replaced by —CO—, —CS—, —CONBR^{E}—, —CONBR^{E}NBR^{E}—, —CO₂—, —OCO—, —NBR^{E}COᵣ, —O—, —NBR^{E}CONBR^{E}—, —OCONBR^{E}—, —NBR^{E}NBR^{E}—, —NBR^{E}CO—, —S—, —SO—, —SO₂—, —NBR^{E}—, —SO₂NBR^{E}—, —NBR^{E}SO₂—, or —NBR^{E}SO₂NBR^{E}—. Each BR₉ is hydrogen, BR^{E}, halo, —OH, —NH₂, —NO₂, —CN, —CF₃, or —OCF₃. Each BR^{E} is independently a C₁₋₈ aliphatic group, a cycloaliphatic, a heterocycloaliphatic, an aryl, or a heteroaryl, each of which is optionally substituted with 1, 2, or 3 of BR^{A}. Each BR^{A} is —Z^{A}BR₅, wherein each Z^{A} is independently a bond or an optionally substituted branched or straight C₁₋₆ aliphatic chain wherein up to two carbon units of Z^{A} are optionally and independently replaced by —CO—, —CS—, —CONBR^{B}—, —CONBR^{B}NBR^{B}—, —CO₂—, —OCO—, —NBR^{B}CO₂—, —O—, —NBR^{B}CONBR^{B}—, —OCONBR^{B}—, —NBR^{B}NBR^{B}—, —NBR^{B}CO—, —S—, —SO—, —SO₂—, —NBR^{B}—, —SO₂NBR^{B}—, —NBR^{B}SO₂—, or —NBR^{B}SO₂NBR^{B}—. Each BR₅ is independently BR^{B}, halo, —B(OH)₂, —OH, —NH₂, —NO₂, —CN, —CF₃, or —OCF₃. Each BR^{B} is independently hydrogen, an optionally substituted C₁₋₈ aliphatic group, an optionally substituted cycloaliphatic, an optionally substituted heterocycloaliphatic, an optionally substituted aryl, or an optionally substituted heteroaryl.

In several embodiments, BR₁ is —Z^{E}BR₉, wherein each Z^{E} is independently a bond or an optionally substituted branched or straight C₁₋₆ aliphatic chain wherein up to two carbon units of Z^{E} are optionally and independently replaced by —CO—, —CONBR^{E}—, —CO₂—, —O—, —S—, —SO—, —SO₂—, —NBR^{E}—, or —SO₂NBR^{E}—. Each BR₉ is hydrogen, BR^{E}, halo, —OH, —NH₂, —CN, —CF₃, or —OCF₃. Each BR^{E} is independently an optionally substituted group selected from C₁₋₈ aliphatic group, cycloaliphatic, heterocycloaliphatic, aryl, and heteroaryl. In one embodiment, Z^{E} is a bond. In one embodiment, Z^{E} is a straight C₁₋₆ aliphatic chain, wherein one carbon unit of Z_{E} is optionally replaced by —CO—, —CONBR^{E}—, —CO₂—, —O—, or —NBR^{E}—. In one embodiment, Z^{E} is a C₁₋₆ alkyl chain. In one embodiment, Z^{E} is —CH₂—. In one embodiment, Z^{E} is —CO—. In one embodiment, Z^{E} is —CO₂—. In one embodiment, Z^{E} is —CONBR^{E}—.

In some embodiments, BR₉ is H, —NH₂, hydroxy, —CN, or an optionally substituted group selected from C₁₋₅ aliphatic, C₃₋₈ cycloaliphatic, 3-8 membered heterocycloaliphatic, C₆₋₁₀ aryl, and 5-10 membered heteroaryl. In one embodiment, BR₉ is H. In one embodiment, BR₉ is hydroxy. Or, BR₉ is —NH₂. Or, BR₉ is —CN. In some embodiments, BR₉ is an optionally substituted 3-8 membered heterocycloaliphatic, having 1, 2, or 3 ring members independently selected from nitrogen (including NH and NBR^{X}), oxygen, and sulfur (including S, SO, and SO₂). In one embodiment, BR₉ is an optionally substituted five membered heterocycloaliphatic with one nitrogen (including NH and NBR^{X}) ring member. In one embodiment, BR₉ is an optionally substituted pyrrolidin-1-yl. Examples of said optionally substituted pyrrolidin-1-yl include pyrrolidin-1-yl and 3-hydroxy-pyrrolidin-1-yl. In one embodiment, R₉ is an optionally substituted six membered heterocycloaliphatic with two heteroatoms independently selected from nitrogen (including NH and NBR^{X}) and oxygen. In one embodiment, BR₉ is morpholin-4-yl. In some embodiments, BR₉ is an optionally substituted 5-10 membered heteroaryl. In one embodiment, BR₉ is an optionally substituted 5 membered heteroaryl, having 1, 2, 3, or 4 ring members independently selected from nitrogen (including NH and NBR^{X}), oxygen, and sulfur (including S, SO, and SO₂). In one embodiment, BR₉ is 1H-tetrazol-5-yl.

In one embodiment, one BR₁ is Z^{E}BR₉; wherein Z^{E} is CH₂ and BR₉ is 1H-tetrazol-5-yl. In one embodiment, one BR₁ is Z^{E}BR₉; wherein Z^{E} is CH₂ and BR₉ is morpholin-4-yl. In one embodiment, one R₁ is Z^{E}BR₉; wherein Z^{E} is CH₂ and BR₉ is pyrrolidin-1-yl. In one embodiment, one BR₁ is Z^{E}BR₉; wherein Z^{E} is CH₂ and BR₉ is 3-hydroxy-pyrrolidin-1-yl. In one embodiment, one BR₁ is Z^{E}BR₉; wherein Z^{E} is CO and BR₉ is 3-hydroxy-pyrrolidin-1-yl.

In some embodiments, BR₁ is selected from CH₂OH, COOH, CH₂OCH₃, COOCH₃, CH₂NH₂, CH₂NHCH₃, CH₂CN, CONHCH₃, CH₂CONH₂, CH₂OCH₂CH₃, CH₂N(CH₃)₂, CON(CH₃)₂, CH₂NHCH₂CH₂OH, CH₂NHCH₂CH₂COOH, CH₂OCH(CH₃)₂, CONHCH(CH₃)CH₂OH, or CONHCH(tert-butyl)CH₂OH.

In several embodiments, BR₁ is halo, or BR₁ is C₁₋₆ aliphatic, aryl, heteroaryl, alkoxy, cycloaliphatic, heterocycloaliphatic, each of which is optionally substituted with 1, 2, or 3 of BR^{A}; or BR₁ is halo; wherein each BR^{A} is —Z^{A}BR₅, each Z^{A} is independently a bond or an optionally substituted branched or straight C₁₋₆ aliphatic chain wherein up to two carbon units of Z^{A} are optionally and independently replaced by —CO—, —CS—, —CONBR^{B}—, —CONBR^{B}1NBR^{B}—, —CO₂—, —OCO—, —NBR^{B}CO₂—, —O—, —NBR^{B}CONBR^{B}—, —OCONBR^{B}—, —NBR^{B}NBR^{B}—, —NBR^{B}CO—, —S—, —SO—, —SO₂—, —NBR^{B}—, —SO₂NBR^{B}—, —NBR^{B}SOᵣ, or —NBR^{B}SO₂NBR^{B}—; each BR₅ is independently BR^{B}, halo, —B(OH)₂, —OH, —NH₂, —NO₂, —CN, —CF₃, or —OCF₃; and each BR^{B} is hydrogen, optionally substituted C₁₋₄ aliphatic, optionally substituted C₃₋₆ cycloaliphatic, optionally substituted heterocycloaliphatic, optionally substituted phenyl, or optionally substituted heteroaryl.

In some embodiments, Z^{A} is independently a bond or an optionally substituted branched or straight C₁₋₆ aliphatic chain wherein up to two carbon units of Z^{A} are optionally and independently replaced by —CO—, —CS—, —CONBR^{B}—, —CONBR^{B}NBR^{B}—, —OCO—, —NBR^{B}CO₂—, —O—, —NBR^{B}CONBR^{B}—, —OCONBR^{B}—, —NBR^{B}NBR^{B}—, —NBR^{B}CO—, —S—, —SO—, —SO₂—, —SO₂NBR^{B}—, —NBR^{B}SO₂—, or —NBR^{B}SO₂NBR^{B}—. In one embodiment, Z^{A} is a bond. In some embodiments, Z^{A} is an optionally substituted straight or branched C₁₋₆ aliphatic chain wherein up to two carbon unites of Z^{A} are optionally and independently replaced by —CO—, —CS—, —CONBR^{B}—, —CONBR^{B}NBR^{B}—, —NBR^{B}CO₂—, —O—, —NBR^{B}CONBR^{B}—, —OCONBR^{B}—, —NBR^{B}NBR^{B}—, —NBR^{B}CO—, —S—, —SO—, —SO₂—, —SO₂NBR^{B}—, —NBR^{B}SO₂—, or —NBR^{B}SO₂NBR^{B}—. In one embodiment, Z^{A} is an optionally substituted straight or branched C₁₋₆ alkyl chain wherein up to two carbon units of Z^{A} is optionally replaced by —O—, —NHC(O)—, —C(O)NBR^{B}—, —SO₂—, —NHSO₂—, —NHC(O)—, —SO—, —NBR^{B}SO₂—, —SO₂NH—, —SO₂NBR^{B}—, —NH—, or —C(O)O—. In one embodiment, Z^{A} is an optionally substituted straight or branched C₁₋₆ alkyl chain wherein one carbon unit of Z^{A} is optionally replaced by —O—, —NHC(O)—, —C(O)NBR^{B}—, —NHSO₂—, —NHC(O)—, —SO—, —NBR^{B}SO₂—, —SO₂NH—, —SO₂NBR^{B}—, —NH—, or —C(O)O—. In one embodiment, Z^{A} is an optionally substituted straight or branched C₁₋₆ alkyl chain wherein one carbon unit of Z^{A} is optionally replaced by —CO—, —CONBR^{B}—, —CO₂—, —O—, —NBR^{B}CO—, —SO₂—, —SO₂NBR^{B}—, or —NBR^{B}SO₂—. In one embodiment, Z^{A} is an optionally substituted straight or branched C₁₋₆ alkyl chain wherein one carbon unit of Z^{A} is optionally replaced by —SO₂—, —CONBR^{B}—, or —SO₂NBR^{B}—. In one embodiment, Z^{A} is —CH₂— or —CH₂CH₂—. In one embodiment, Z^{A} is an optionally substituted straight or branched C₁₋₆ alkyl chain wherein one carbon unit of Z^{A} is optionally replaced by —CO—, —CONBR^{B}—, —CO₂—, —O—, —NHCO—, —SO—, —SO₂NBR^{B}—, or —NBR^{B}SO₂—. In some embodiments, Z^{A} is —CO₂—, —CH₂CO₂—, —CH₂CH₂CO₂—, —CH(NH₂)CH₂CO₂—, or —CH(CH₃)CH₂CO₂—. In some embodiments, Z^{A} is —CONH—, —NHCO—, or —CON(CH₃)—. In some embodiments, Z^{A} is —O—. Or, Z^{A} is —SO—, —SO₂NH—, or —SO₂N(CH₃). In one embodiment, Z^{A} is an optionally substituted branched or straight C₁₋₆ aliphatic chain wherein one carbon unit of Z^{A} is optionally replaced by —SO₂—.

In some embodiments, BR₅ is H, F, Cl, —B(OH)₂, —OH, —NH₂, —CF₃, —OCF₃, or —CN. In one embodiment, BR₅ is H. Or, BR₅ is F. Or, BR₅ is Cl. Or, BR₅ is —B(OH)₂. Or, BR₅ is —OH. Or, BR₅ is —NH₂. Or, BR₅ is —CF₃. Or, BR₅ is —OCF₃. Or, BR₅ is —CN.

In some embodiments, BR₅ is an optionally substituted C₁₋₄ aliphatic. In one embodiment, BR₅ is an optionally substituted C₁₋₄ alkyl. In one embodiment, BR₅ is methyl, ethyl, iso-propyl, or tert-butyl. In one embodiment, BR₅ is an optionally substituted aryl. In one embodiment, BR₅ is an optionally substituted phenyl. In some embodiments, BR₅ is an optionally substituted heteroaryl or an optionally substituted heterocycloaliphatic. In some embodiments, BR₅ is an optionally substituted heteroaryl. In one embodiment, BR₅ is an optionally substituted monocyclic heteroaryl, having 1, 2, 3, or 4 ring members optionally and independently replaced with nitrogen (including NH and NBR^{X}), oxygen or sulfur (including S, SO, and SO₂). In one embodiment, BR₅ is an optionally substituted 5 membered heteroaryl. In one embodiment, BR₅ is 1H-tetrazol-5-yl. In one embodiment, BR₅ is an optionally substituted bicyclic heteroaryl. In one embodiment, BR₅ is a 1,3-dioxoisoindolin-2-yl. In some embodiments, BR₅ is an optionally substituted heterocycloaliphatic having 1 or 2 nitrogen (including NH and NBR^{X}) atoms and BR₅ attaches directly to —SO₂— via one ring nitrogen.

In some embodiments, two occurrences of BR^{A}, taken together with carbon atoms to which they are attached, form an optionally substituted 3-8 membered saturated, partially unsaturated, or aromatic ring, having up to 4 ring members optionally and independently replaced with nitrogen (including NH and NBR^{X}), oxygen, or sulfur (including S, SO, and SO₂). In some embodiments, two occurrences of BR^{A}, taken together with carbon atoms to which they are attached, form C₄₋₈ cycloaliphatic ring optionally substituted with 1, 2, or 3 substituents independently selected from oxo, ═NBR^{B}, ═N—N(BR^{B})₂, halo, —CN, —CO₂, —CF₃, —OCF₃, —OH, —SBR^{B}, —S(O)BR^{B}, —SO₂BR^{B}, —NH₂, —NHB^{B}, —N(BR^{B})₂, —COOH, —COOBR^{B}, —OBR^{B}, or BR^{B}. In one embodiment, said cycloaliphatic ring is substituted with oxo. In one embodiment, said cycloaliphatic ring is

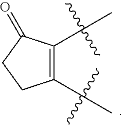 [SMILES: *C1=C(*)C(=O)CC1]

In some embodiments, two occurrences of BR^{A}, taken together with carbon atoms to which they are attached, form an optionally substituted 5-8 membered heterocycloaliphatic ring, having up to 4 ring members optionally and independently replaced with nitrogen (including NH and NBR^{X}), oxygen, or sulfur (including S, SO, and SO₂). In some embodiments, two occurrences of BR^{A}, taken together with carbon atoms to which they are attached, form a 5 or 6 membered heterocycloaliphatic ring, optionally substituted with 1, 2, or 3 substituents independently selected from oxo, ═NBR^{B}, ═N—N(BR^{B})₂, halo, CN, CO₂, CF₃, OCF₃, OH, SBR^{B}, S(O)BR^{B}, SO₂BR^{B}, NH₂, NHBR^{B}, N(BR^{B})₂, COOH, COOBR^{B}, OBR^{B}, or BR^{B}. In some embodiments, said heterocycloaliphatic ring is selected from:

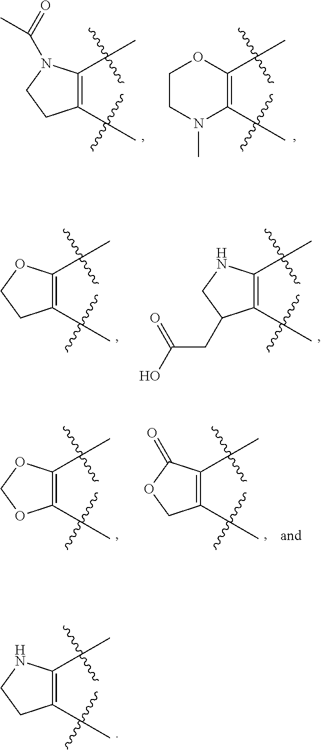

In some embodiments, two occurrences of BR^{A}, taken together with carbon atoms to which they are attached, form an optionally substituted C₆₋₁₀ aryl. In some embodiments, two occurrences of BR^{A}, taken together with carbon atoms to which they are attached, form a 6 membered aryl, optionally substituted with 1, 2, or 3 substituents independently selected from halo, —CN, —CO₂, —CF₃, —OH, —SBR^{B}, —S(O)BR^{B}, —SO₂BR^{B}, —NH₂, —NHBR^{B}, —N(BR^{B})₂, —COOH, —COOBR^{B}, —OBR^{B}, or BR^{B}. In some embodiments, said aryl is

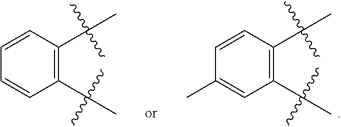

In some embodiments, two occurrences of BR^{A}, taken together with carbon atoms to which they are attached, form an optionally substituted 5-8 membered heteroaryl, having up to 4 ring members optionally and independently replaced with nitrogen (including NH and NBR^{X}), oxygen, or sulfur (including S, SO, and SO₂). In some embodiments, two occurrences of BR^{A}, taken together with carbon atoms to which they are attached, form a 5 or 6 membered heteroaryl, optionally substituted with 1, 2, or 3 substituents independently selected from halo, CN, CO₂, CF₃, OCF₃, OH, SBR^{B}, S(O)BR^{B}, SO₂BR^{B}, NH₂, NHBR^{B}, N(BR^{B})₂, COOH, COOBR^{B}, OBR^{B}, or BR^{B}. In some embodiments, said heteroaryl is selected from:

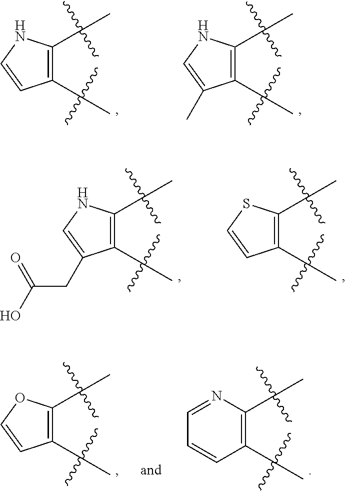

In some embodiments, one BR₁ is aryl or heteroaryl, each optionally substituted with 1, 2, or 3 of BR^{A}, wherein BR^{A} is defined above.

In several embodiments, one BR₁ is carboxy [e.g., hydroxycarbonyl or alkoxycarbonyl], amido [e.g., aminocarbonyl], amino, halo, cyano, or hydroxy.

In several embodiments, BR₁ is:

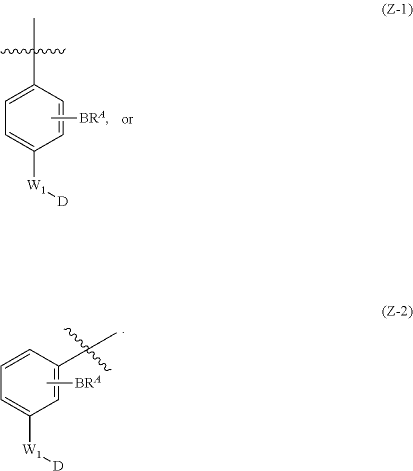

wherein

W₁ is —C(O)—, —SO₂—, —NHC(O)—, or —CH₂—;

D is H, hydroxy, or an optionally substituted group selected from aliphatic, cycloaliphatic, alkoxy, and amino; and

BR^{A} is defined above.

In several embodiments, W₁ is —C(O)—. Or, W₁ is —SO₂—. Or, W₁ is —NHC(O)—. Or, W₁ is —CH₂—.

In several embodiments, D is OH. Or, D is an optionally substituted C₁₋₆ aliphatic or an optionally substituted C₃-C₈ cycloaliphatic. Or, D is an optionally substituted alkoxy. Or, D is an optionally substituted amino.

In several examples, D is

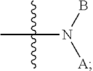

wherein each of A and B is independently H, an optionally substituted C₁₋₆ aliphatic, an optionally substituted C₃-C₈ cycloaliphatic, an optionally substituted 3-8 membered heterocycloaliphatic, acyl, sulfonyl, alkoxy or

A and B, taken together, form an optionally substituted 3-7 membered heterocycloaliphatic ring.

In some embodiments, A is H. In some embodiments, A is an optionally substituted C₁₋₆ aliphatic. In several examples, A is an optionally substituted C₁₋₆ alkyl. In one example, A is methyl. Or, A is ethyl. Or, A is n-propyl. Or, A is iso-propyl. Or, A is 2-hydroxyethyl. Or, A is 2-methoxyethyl.

In several embodiments, B is C₁₋₆ straight or branched alkyl, optionally substituted with 1, 2, or 3 substituents each independently selected from halo, oxo, CN, hydroxy, or an optionally substituted group selected from alkyl, alkenyl, hydroxyalkyl, alkoxy, alkoxyalkyl, cycloaliphatic, amino, heterocycloaliphatic, aryl, and heteroaryl. In several embodiments, B is substituted with 1, 2, or 3 substituents each independently selected from halo, oxo, CN, C₁₋₆ alkyl, C₂₋₆ alkenyl, hydroxy, hydroxy-(C₁₋₆)alkyl, (C₁₋₆)alkoxy, (C₁₋₆)alkoxy(C₁₋₆)alkyl, NH₂, NH(C₁₋₆ alkyl), N(C₁₋₆ alkyl)₂, C₃₋₈ cycloaliphatic, NH(C₃₋₈ cycloaliphatic), N(C₁₋₆ alkyl)(C₃₋₈ cycloaliphatic), N(C₃₋₈ cycloaliphatic)₂, 3-8 membered heterocycloaliphatic, phenyl, and 5-10 membered heteroaryl. In one example, said substituent is oxo. Or, said substituent is optionally substituted (C₁₋₆) alkoxy. Or, is hydroxy. Or, is NH₂. Or, is NHCH₃. Or, is NH(cyclopropyl). Or, is NH(cyclobutyl). Or, is N(CH₃)₂. Or, is CN. In one example, said substituent is optionally substituted phenyl. In some embodiments, B is substituted with 1, 2, or 3 substituents each independently selected from an optionally substituted C₃₋₈ cycloaliphatic or 3-8 membered heterocycloaliphatic. In one example, said substituent is an optionally substituted group selected from cyclopropyl, cyclobutyl, cyclopentyl, cyclohexyl, cycloheptyl, cyclohexenyl, morpholin-4-yl, pyrrolidin-1-yl, pyrrolidin-2-yl, 1,3-dioxolan-2-yl, and tetrahydrofuran-2-yl. In some embodiments, B is substituted with 1, 2, or 3 substituents each independently selected from an optionally substituted 5-8 membered heteroaryl. In one example, said substituent is an optionally substituted group selected from pyridyl, pyrazyl, 1H-imidazol-1-yl, and 1H-imidazol-5-yl.

In some embodiments, B is C₃-C₈ cycloaliphatic optionally substituted with 1, 2, or 3 substituents independently selected from halo, oxo, alkyl, hydroxy, hydroxyalkyl, alkoxy, alkoxyalkyl, dialkyamino, or an optionally substituted group selected from cycloaliphatic, heterocycloaliphatic, aryl, and heteroaryl. In several examples, B is an optionally substituted C₃₋₈ cycloalkyl. In one embodiment, B is cyclopropyl. Or, B is cyclobutyl. Or, B is cyclopentyl. Or, B is cyclohexyl. Or, B is cycloheptyl.

In some embodiments, B is 3-8 membered heterocycloaliphatic optionally substituted with 1, 2, or 3 substituents independently selected from oxo, alkyl, hydroxy, hydroxyalkyl, alkoxy, alkoxyalkyl, dialkyamino, or an optionally substituted group selected from cycloaliphatic, heterocycloaliphatic, aryl, and heteroaryl. In one example, B is 3-oxo-isoxazolid-4-yl.

In several embodiments, A is H and B is an optionally substituted C₁₋₆ aliphatic. In several embodiments, B is substituted with 1, 2, or 3 substituents. Or, both, A and B, are H. Exemplary substituents on B include halo, oxo, alkyl, hydroxy, hydroxyalkyl, alkoxy, alkoxyalkyl, dialkyamino, or an optionally substituted group selected from cycloaliphatic, heterocycloaliphatic, aryl, and heteroaryl.

In several embodiments, A is H and B is an optionally substituted C₁₋₆ aliphatic. Exemplary substituents include oxo, alkyl, hydroxy, hydroxyalkyl, alkoxy, alkoxyalkyl, and an optionally substituted heterocycloaliphatic.

In several embodiments, A and B, taken together, form an optionally substituted 3-7 membered heterocycloaliphatic ring. In several examples, the heterocycloaliphatic ring is optionally substituted with 1, 2, or 3 substituents. Exemplary such rings include pyrrolidinyl, piperidinyl, morpholinyl, piperazinyl, oxazolidin-3-yl, and 1,4-diazepan-1-yl. Exemplary said substituents on such rings include halo, oxo, alkyl, aryl, heteroaryl, hydroxy, hydroxyalkyl, alkoxy, alkoxyalkyl, acyl (e.g., alkylcarbonyl), amino, amido, and carboxy. In some embodiments, each of said substituents is independently halo, oxo, alkyl, aryl, heteroaryl, hydroxy, hydroxyalkyl, alkoxy, alkoxyalkyl, amino, amido, or carboxy. In one embodiment, the substituent is oxo, F, Cl, methyl, ethyl, iso-propyl, 2-methoxyethyl, hydroxymethyl, methoxymethyl, aminocarbonyl, —COOH, hydroxy, acetyl, or pyridyl.

In several embodiments, BR₁ is:

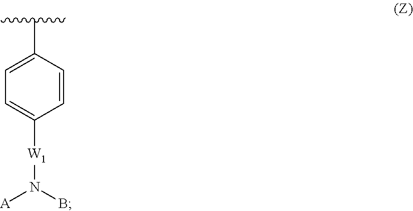

wherein:

W₁ is —C(O)—, —SO₂—, —NHC(O)—, or —CH₂—;

Each of A and B is independently H, an optionally substituted C₁₋₆ aliphatic, an optionally substituted C₃-C₈ cycloaliphatic; or

A and B, taken together, form an optionally substituted 4-7 membered heterocycloaliphatic ring.

In several examples, BR₁ is selected from any one of the exemplary compounds in Table II.B-1.

### Substituent BR₂

Each BR₂ is hydrogen, or optionally substituted C₁₋₆ aliphatic, C₃₋₆ cycloaliphatic, phenyl, or heteroaryl.

In several embodiments, BR₂ is a C₁₋₆ aliphatic that is optionally substituted with 1, 2, or 3 halo, C₁₋₂ aliphatic, or alkoxy. In several examples, BR₂ is substituted or unsubstituted methyl, ethyl, propyl, or butyl.

In several embodiments, BR₂ is hydrogen.

### Ring A

Ring A is an optionally substituted cycloaliphatic or an optionally substituted heterocycloaliphatic where the atoms of ring A adjacent to C* are carbon atoms. In several embodiments, ring A is C₃₋₇ cycloaliphatic or 3-8 membered heterocycloaliphatic, each of which is optionally substituted with 1, 2, or 3 substituents.

In several embodiments, ring A is optionally substituted with 1, 2, or 3 of —Z^{B}BR₇, wherein each Z^{B} is independently a bond, or an optionally substituted branched or straight C₁₋₄ aliphatic chain wherein up to two carbon units of Z^{B} are optionally and independently replaced by —CO—, —CS—, —CONBR^{B}—, —CONBR^{B}NBR^{B}—, —CO₂—, —OCO—, —NBR^{B}CO₂—, —O—, —NBR^{B}CONBR^{B}—, —OCONBR^{B}—, —NBR^{B}NBR^{B}—, —NBR^{B}CO—, —S—, —SO—, —SO₂—, —NBR^{B}—, —SO₂NBR^{B}—, —NBR^{B}SO₂—, or —NBR^{B}SO₂NBR^{B}—; each R₇ is independently BR^{B}, halo, —OH, —NH₂, —NO₂, —CN, or —OCF₃; and each BR^{B} is independently hydrogen, an optionally substituted C₁₋₈ aliphatic group, an optionally substituted cycloaliphatic, an optionally substituted heterocycloaliphatic, an optionally substituted aryl, or an optionally substituted heteroaryl.

In several embodiments, ring A is a C₃₋₇ cycloaliphatic or a 3-8 membered heterocycloaliphatic, each of which is optionally substituted with 1, 2, or 3 substituents.

In several embodiments, ring A is a 3, 4, 5, or 6 membered cycloaliphatic that is optionally substituted with 1, 2, or 3 substituents. In several examples, ring A is an optionally substituted cyclopropyl group. In several alternative examples, ring A is an optionally substituted cyclobutyl group. In several other examples, ring A is an optionally substituted cyclopentyl group. In other examples, ring A is an optionally substituted cyclohexyl group. In more examples, ring A is an unsubstituted cyclopropyl.

In several embodiments, ring A is a 5, 6, or 7 membered optionally substitute heterocycloaliphatic. For example, ring A is an optionally substituted tetrahydropyranyl group.

### Substituent BR₄

Each BR₄ is independently an optionally substituted aryl or heteroaryl.

In several embodiments, BR₄ is an aryl having 6 to 10 members (e.g., 7 to 10 members) optionally substituted with 1, 2, or 3 substituents. Examples of BR₄ are optionally substituted benzene, naphthalene, or indene. Or, examples of BR₄ can be optionally substituted phenyl, optionally substituted naphthyl, or optionally substituted indenyl.

In several embodiments, BR₄ is an optionally substituted heteroaryl. Examples of BR₄ include monocyclic and bicyclic heteroaryl, such a benzofused ring system in which the phenyl is fused with one or two C4-8 heterocycloaliphatic groups.

In some embodiments, BR₄ is an aryl or heteroaryl, each optionally substituted with 1, 2, or 3 of —ZCBR8. Each ZC is independently a bond or an optionally substituted branched or straight C1-6 aliphatic chain wherein up to two carbon units of ZC are optionally and independently replaced by —CO—, —CS—, —CONBRC—, —CONBRCNBRC—, —CO2-, —OCO—, —NBRCCO2-, —O—, —NBRCCONBRC—, —OCONBRC—, —NBRCNBRC—, —NBRCCO—, —S—, —SO—, —SO2-, —NBRC—, —SO2NBRC—, —NBRCSO2-, or —NBRCSO2NBRC—. Each BR8 is independently BRC, halo, —OH, —NH2, —NO2, —CN, or —OCF3. Each BRC is independently hydrogen, an optionally substituted C₁₋₈ aliphatic group, an optionally substituted cycloaliphatic, an optionally substituted heterocycloaliphatic, an optionally substituted aryl, or an optionally substituted heteroaryl. In one embodiment, BR₄ is an aryl optionally substituted with 1, 2, or 3 of ZCBR8. In one embodiment, BR₄ is an optionally substituted phenyl.

In several embodiments, BR₄ is a heteroaryl optionally substituted with 1, 2, or 3 substituents. Examples of BR4 include optionally substituted benzo[d][1,3]dioxole or 2,2-difluoro-benzo[d][1,3]dioxole.

In some embodiments, two occurrences of —ZCBR8, taken together with carbons to which they are attached, form a 4-8 membered saturated, partially saturated, or aromatic ring with up to 3 ring atoms independently selected from the group consisting of O, NH, NBRC, and S (including S, SO, and SO2); wherein BRC is defined herein.

In several embodiments, BR₄ is one selected from

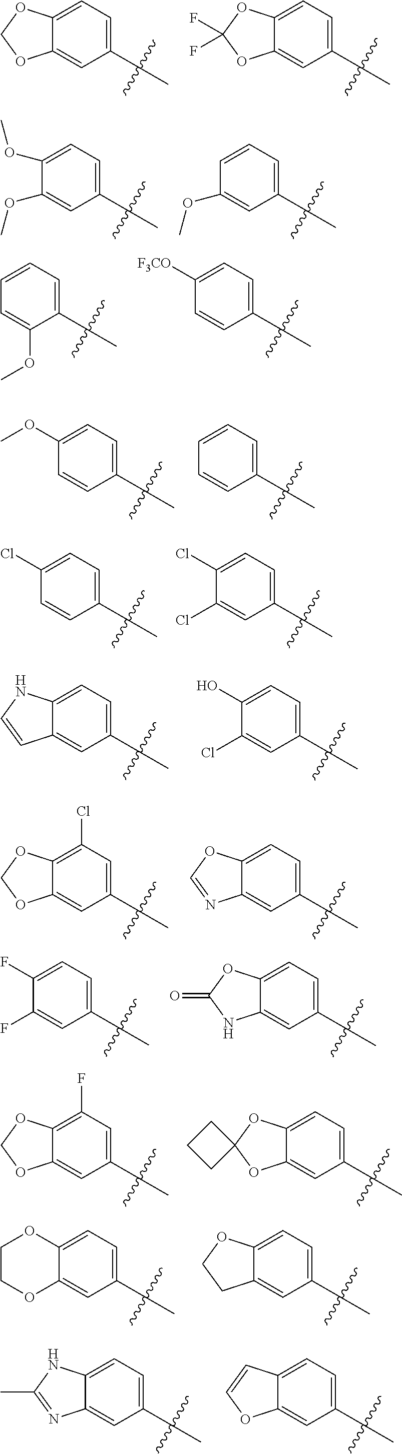

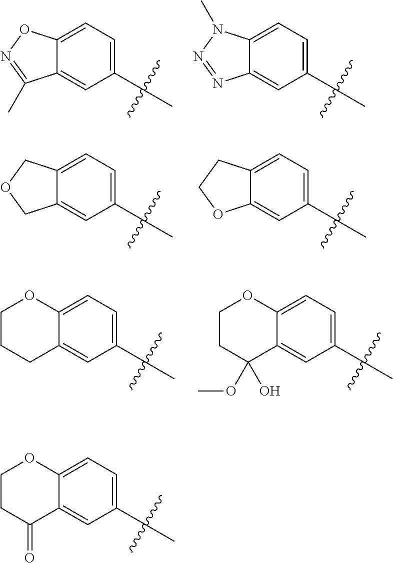

II.B.2 Compounds of Formulas B1 and B2

Another aspect of the present invention includes compounds of formula B1a:

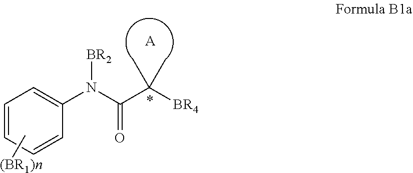

or a pharmaceutically acceptable salt thereof, wherein BR₂, BR₄, and n have been defined in Formula B.

Each BR₁ is independently aryl, monocyclic heteroaryl or indolizinyl, indolyl, isoindolyl, 3H-indolyl, indolinyl, benzo[b]furanyl, benzo[b]thiophenyl, 1H-indazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, imidazo[1,2-a]pyridinyl, or benzo[d]oxazolyl, each of which is optionally substituted with 1, 2, or 3 of BRA; or BR1 is independently methyl, trifluoromethyl, or halo. In one embodiment, BR₁ is an optionally substituted imidazo[1,2-a]pyridine-2-yl. In one embodiment, BR₁ is an optionally substituted oxazolo[4,5-b]pyridine-2-yl. In one embodiment, BR₁ is an optionally substituted 1H-pyrrolo[2,3-b]pyrid-6-yl. In one embodiment, BR₁ is an optionally substituted benzo[d]oxazol-2-yl. In one embodiment, BR1 is an optionally substituted benzo[d]thiazol-2-yl.

In some embodiments, BR₁ is a monocyclic aryl or a monocyclic heteroaryl, each is optionally substituted with 1, 2, or 3 of BRA. In some embodiments, BR₁ is substituted or unsubstituted phenyl. In one embodiment, BR₁ is substituted or unsubstituted pyrid-2-yl. In some embodiments, BR₁ is pyrid-3-yl, pyrid-4-yl, thiophen-2-yl, thiophen-3-yl, 1H-pyrrol-2-yl, 1H-pyrrol-3-yl, 1H-imidazol-5-yl, 1H-pyrazol-4-yl, 1H-pyrazol-3-yl, thiazol-4-yl, furan-3-yl, furan-2-yl, or pyrimidin-5-yl, each of which is optionally substituted. In some embodiments, BR₁ is phenyl, pyrid-2-yl, pyrid-3-yl, pyrid-4-yl, thiophen-2-yl, thiophen-3-yl, 1H-pyrrol-2-yl, 1H-pyrrol-3-yl, 1H-imidazol-5-yl, 1H-pyrazol-4-yl, 1H-pyrazol-3-yl, thiazol-4-yl, furan-3-yl, furan-2-yl, or pyrimidin-5-yl, each of which is optionally substituted with 1, 2, or 3 substituents independently selected from CN, or a group chosen from C1-6 alkyl, carboxy, alkoxy, halo, amido, acetoamino, and aryl, each of which is further optionally substituted.

Each BRA is —ZABR5, wherein each ZA is independently a bond or an optionally substituted branched or straight C1-6 aliphatic chain wherein up to two carbon units of ZA are optionally and independently replaced by —CS—, —CONBRB—, —CONBRBNBRB—, —CO2-, —NBRBCO2-, —NBRBCONBRB—, —NBRBNBRB—, —NBRBCO—, —S—, —SO—, —SO₂—, —NBRB—, —SO2NBRB—, —NBRBSO2-, or —NBRBSO2NBRB—.

Each BR₅ is independently BRB, halo, —OH, —NH2, —NO2, —CN, or —OCF3.

Each BRB is hydrogen, an optionally substituted C1-4 aliphatic, an optionally substituted C3-6 cycloaliphatic, an optionally substituted heterocycloaliphatic, an optionally substituted phenyl, or an optionally substituted heteroaryl.

Ring A is an optionally substituted cycloaliphatic, an optionally substituted 5 membered heterocycloaliphatic having 1, 2, or 3 heteroatoms independently selected from nitrogen (including NH and NBRX), oxygen, or sulfur (including S, SO, and SO2); an optionally substituted 6 membered heterocycloaliphatic having 1 heteroatom selected from O and S (including S, SO, and SO2); a piperidinyl optionally substituted with halo, aliphatic, aminocarbonyl, aminocarbonylaliphatic, aliphatic carbonyl, aliphaticsulfonyl, aryl, or combinations thereof; or an optionally substituted 7-8 membered heterocycloaliphatic having 1, 2, or 3 heteroatoms independently selected from nitrogen (including NH and NBRX), oxygen, or sulfur (including S, SO, and SO2).

In some embodiments, one BR₁ attached to the 3- or 4-position of the phenyl ring is an aryl or heteroaryl optionally substituted with 1, 2, or 3 of BRA, wherein BRA is —ZABR5; in which each ZA is independently a bond or an optionally substituted branched or straight C1-6 aliphatic chain wherein up to two carbon units of ZA are optionally and independently replaced by —CO—, —CS—, —CONRB—, —CONBRBNBRB—, —CO2-, —OCO—, —NBRBCO2-, —O—, —NBRBCONBRB—, —OCONBRB—, —NBRBNBRB—, —NBRBCO—, —S—, —SO—, —SO2-, —NBRB—, —SO2NBRB—, —NBRBSO2-, or —NBRBSO2NBRB—; each BR5 is independently BRB, halo, —OH, —NH2, —NO2, —CN, or —OCF3; and each BRB is independently hydrogen, an optionally substituted C1-8 aliphatic group, an optionally substituted cycloaliphatic, an optionally substituted heterocycloaliphatic, an optionally substituted aryl, or an optionally substituted heteroaryl.

In some embodiments, one BR₁ attached to the 3- or 4-position of the phenyl ring is a phenyl optionally substituted with 1, 2, or 3 of BRA.

In some embodiments, one BR₁ attached to the 3- or 4-position of the phenyl ring is a phenyl substituted with one of BRA, wherein BRA is —ZABR5; each ZA is independently a bond or an optionally substituted branched or straight C1-6 aliphatic chain wherein up to two carbon units of ZA are optionally and independently replaced by —O—, —NHC(O)—, —C(O)NBRB—, —SO2-, —NHSO2-, —NHC(O)—, —SO—, —NBRBSO2-, —SO2NH—, —SO2NBRB—, —NH—, or —C(O)O—. In one embodiment, one carbon unit of ZA is replaced by —O—, —NHC(O)—, —C(O)NBRB—, —SO2-, —NHSO2-, —NHC(O)—, —SO—, —NRBSO2-, —SO2NH—, —SO2NBRB—, —NH—, or —C(O)O—. In some embodiments, BR5 is independently an optionally substituted aliphatic, an optionally substituted cycloaliphatic, an optionally substituted heterocycloaliphatic, an optionally substituted aryl, an optionally substituted heteroaryl, hydrogen, or halo.

In some embodiments, one BR₁ attached to the 3- or 4-position of the phenyl ring is heteroaryl optionally substituted with 1, 2, or 3 of BRA. In several examples, one BR₁ attached to the 3- or 4-position of the phenyl ring is a 5 or 6 membered heteroaryl having 1, 2, or 3 heteroatoms independently selected from nitrogen (including NH and NBRX), oxygen or sulfur (including S, SO, and SO2), wherein the heteroaryl is substituted with one of BRA, wherein BRA is —ZABR5; wherein each ZA is independently a bond or an optionally substituted branched or straight C1-6 aliphatic chain wherein up to two carbon units of ZA are optionally and independently replaced by —O—, —NHC(O)—, —C(O)NBRB—, —SO2-, —NHSO2-, —NHC(O)—, —SO—, —NBRBSO2-, —SO2NH—, —SO2NBRB—, —NH—, or —C(O)O—. In one embodiment, one carbon unit of ZA is replaced by —O—, —NHC(O)—, —C(O)NBRB—, —SO2-, —NHSO2-, —NHC(O)—, —SO—, —NBRBSO2-, —SO2NH—, —SO2NBRB—, —NH—, or —C(O)O—. In one embodiment, BR5 is independently an optionally substituted aliphatic, an optionally substituted cycloaliphatic, an optionally substituted heterocycloaliphatic, an optionally substituted aryl, an optionally substituted heteroaryl, hydrogen, or halo.

Another aspect of the present invention includes compounds of Formula B1b:

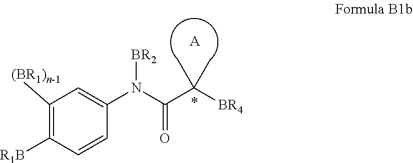

or a pharmaceutically acceptable salt thereof, wherein BR₂, BR₄ and ring A are defined in Formula B.

The BR₁ attached at the para position relative to the amide is an aryl or a heteroaryl optionally substituted with 1, 2, or 3 of BRA; wherein each BRA is —ZABR5, each ZA is independently a bond or an optionally substituted branched or straight C1-6 aliphatic chain wherein up to two carbon units of ZA are optionally and independently replaced by —CO—, —CS—, —CONBRB—, —CONBRBNBRB—, —CO2-, —OCO—, —NBRBCO2-, —O—, —NBRBCONBRB—, —OCONBRB—, —NBRBNBRB—, —NBRBCO—, —S—, —SO—, —SO2-, —NBRB—, —SO2NBRB—, —NBRBSO2-, or —NBRBSO2NBRB—; each BR5 is independently BRB, halo, —OH, —NH2, —NO2, —CN, or —OCF₃; each BRB is hydrogen, an optionally substituted C1-4 aliphatic, an optionally substituted C3-4 cycloaliphatic, an optionally substituted heterocycloaliphatic, an optionally substituted phenyl, or optionally substituted heteroaryl.

The other BR₁ are each independently hydrogen, halo, optionally substituted C1-4 aliphatic, or optionally substituted C1-4 alkoxy.

In several embodiments, the BR₁ attached at the para position relative to the amide is a phenyl optionally substituted with 1, 2, or 3 of BRA and the other BR₁s are each hydrogen. For example, the BR₁ attached at the para position relative to the amide is phenyl optionally substituted with aliphatic, alkoxy, (amino)aliphatic, hydroxyaliphatic, aminosulfonyl, aminocarbonyl, alcoxycarbonyl, (aliphatic)aminocarbonyl, COOH, (aliphatic)aminosulfonyl, or combinations thereof, each of which is optionally substituted. In other embodiments, the BR1 attached at the para position relative to the amide is phenyl optionally substituted with halo. In several examples, the BR1 attached at the para position relative to the amide is phenyl optionally substituted with alkyl, alkoxy, (amino)alkyl, hydroxyalkyl, aminosulfonyl, (alkyl)aminocarbonyl, (alkyl)aminosulfonyl, or combinations thereof, each of which is optionally substituted; or the BR1 attached at the para position relative to the amide is phenyl optionally substituted with halo.

In several embodiments, the BR₁ attached at the para position relative to the amide is an optionally substituted heteroaryl. In other embodiments, the BR₁ attached at the para position relative to the amide is an optionally substituted monocyclic or optionally substituted bicyclic heteroaryl. For example, the BR₁ attached at the para position relative to the amide is a benzo[d]oxazolyl, thiazolyl, benzo[d]thiazolyl, indolyl, or imidazo[1,2-a]pyridinyl, each of which is optionally substituted. In other examples, the BR₁ attached at the para position relative to the amide is a benzo[d]oxazolyl, thiazolyl, benzo[d]thiazolyl, or imidazo[1,2-a]pyridinyl, each of which is optionally substituted with 1, 2, or 3 of halo, hydroxy, aliphatic, alkoxy, or combinations thereof, each of which is optionally substituted.

In several embodiments, each BR₁ not attached at the para position relative to the amide is hydrogen. In some examples, each BR₁ not attached at the para position relative to the amide is methyl, ethyl, propyl, isopropyl, or tert-butyl, each of which is optionally substituted with 1, 2, or 3 of halo, hydroxy, cyano, or nitro. In other examples, each BR₁ not attached at the para position relative to the amide is halo or optionally substituted methoxy, ethoxy, or propoxy. In several embodiments, each BR1 not attached at the para position relative to the amide is hydrogen, halo, —CH3, —OCH3, or —CF3.

In several embodiments, compounds of formula BIb include compounds of formulae B1b1, B1b2, B1b3, or B1b4:

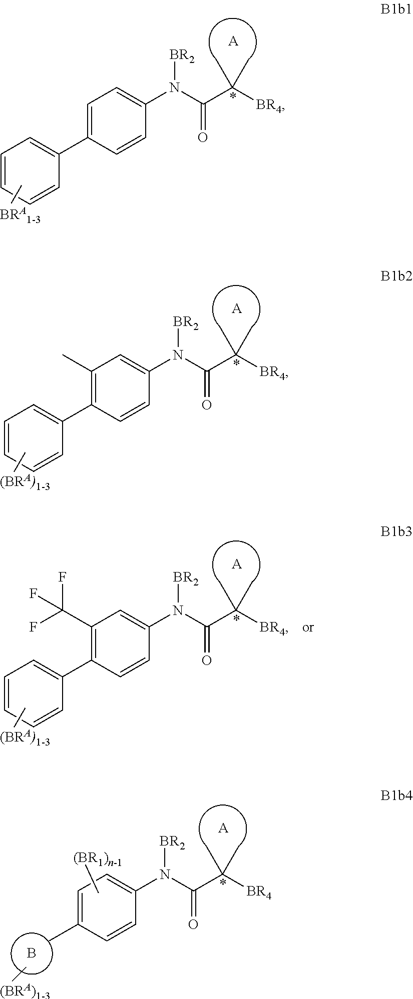

where BRA, BR₁, BR₂, BR₄, and ring A are defined above.

In formula B1b4, ring B is monocyclic or bicyclic heteroaryl that is substituted with 1, 2, or 3 RA; and “n−1” is equal to 0, 1, or 2.

In several embodiments, the BR₁ attached at the para position relative to the amide in formula Ib is an optionally substituted aryl. In several embodiments, the BR₁ attached at the para position relative to the amide is a phenyl optionally substituted with 1, 2, or 3 of BRA. For example, the BR₁ attached at the para position relative to the amide is phenyl optionally substituted with 1, 2, or 3 aliphatic, alkoxy, COOH, (amino)aliphatic, hydroxyaliphatic, aminosulfonyl, (aliphatic)aminocarbonyl, (aliphatic)aminosulfonyl, (((aliphatic)sulfonyl)amino)aliphatic, (heterocycloaliphatic)sulfonyl, heteroaryl, aliphaticsulfanyl, or combinations thereof, each of which is optionally substituted; or BR₁ is optionally substituted with 1-3 of halo.

In several embodiments, the BR₁ attached at the para position relative to the amide in formula Ib is an optionally substituted heteroaryl. In other embodiments BR₁ is an optionally substituted monocyclic or an optionally substituted bicyclic heteroaryl. For example, BR₁ is a pyridinyl, thiazolyl, benzo[d]oxazolyl, or oxazolo[4,5-b]pyridinyl, each of which is optionally substituted with 1, 2, or 3 of halo, aliphatic, alkoxy, or combinations thereof.

In several embodiments, one BR₁ not attached at the para position relative to the amide is halo, optionally substituted C1-4 aliphatic, C1-4 alkoxyC1-4 aliphatic, or optionally substituted C1-4 alkoxy, such as For example, one BR1 not attached at the para position relative to the amide is halo, —CH3, ethyl, propyl, isopropyl, tert-butyl, or —OCF₃.

In several embodiments, compounds of the invention include compounds of formulae B1c1, B1c2, B1c3, B1c4, B1c5, B1c6, B1c7, or B1c8:

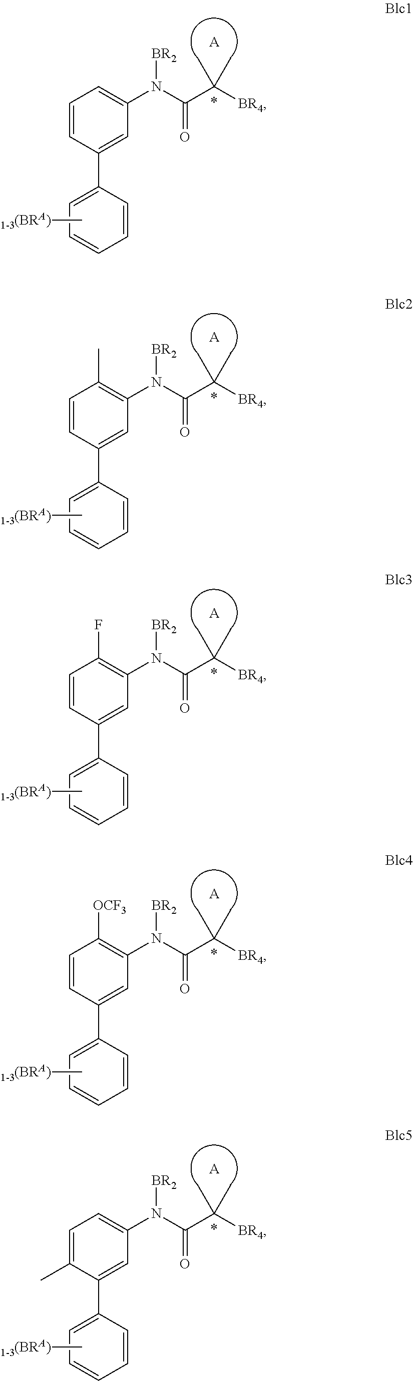

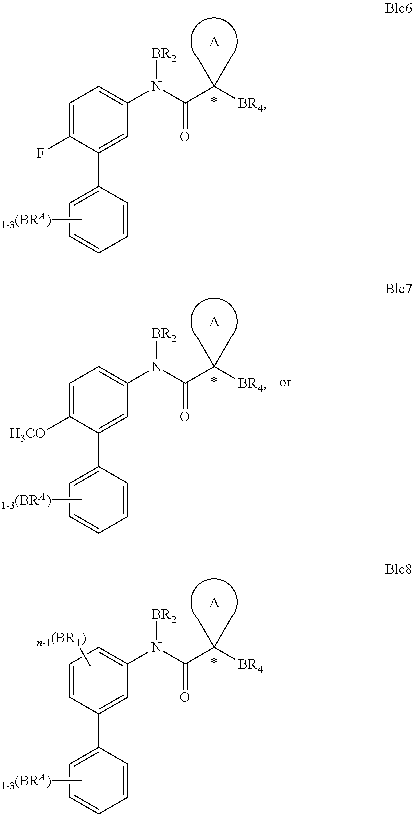

or pharmaceutically acceptable salts, wherein BRA, BR₂, BR₁, BR₄, and ring A are defined above.

In formula B1c8, ring B is monocyclic or bicyclic heteroaryl that is substituted with 1, 2, or 3 BRA; and “n−1” is equal to 0, 1, or 2.

Another aspect of the present invention provides compounds of formula B1d:

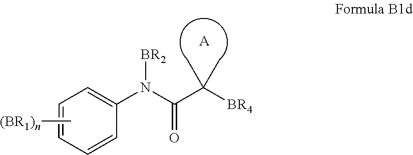

or a pharmaceutically acceptable salt thereof, wherein BR₁, BR₂, BR₄, and n are defined in Formula B.

Ring A is an optionally substituted cycloaliphatic.

In several embodiments, ring A is a cyclopropyl, cyclopentyl, or cyclohexyl, each of which is optionally substituted.

Another aspect of the present invention provides compounds of Formula B1e:

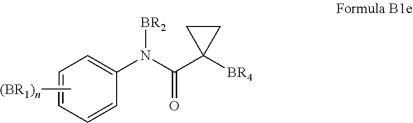

or a pharmaceutically acceptable salt thereof, wherein BR₁, BR₂, and n are defined in Formula B.

BR₄ is an optionally substituted phenyl or an optionally substituted benzo[d][1,3]dioxolyl. In several embodiments, BR₄ is optionally substituted with 1, 2, or 3 of hydrogen, halo, optionally substituted aliphatic, optionally substituted alkoxy, or combinations thereof. In several embodiments, BR₄ is phenyl that is substituted at position 2, 3, 4, or combinations thereof with hydrogen, halo, optionally substituted aliphatic, optionally substituted alkoxy, or combinations thereof. For example, BR₄ is phenyl that is optionally substituted at the 3 position with optionally substituted alkoxy. In another example, BR₄ is phenyl that is optionally substituted at the 3 position with —OCH3. In another example, BR₄ is phenyl that is optionally substituted at the 4 position with halo or substituted alkoxy. A more specific example includes an BR₄ that is phenyl optionally substituted with chloro, fluoro, —OCH3, or —OCF3. In other examples, BR₄ is a phenyl that is substituted at the 2 position with an optionally substituted alkoxy. In more specific examples, BR₄ is a phenyl optionally substituted at the 2 position with —OCH3. In other examples, BR₄ is an unsubstituted phenyl.

In several embodiments, BR₄ is optionally substituted benzo[d][1,3]dioxolyl. In several examples, BR₄ is benzo[d][1,3]dioxolyl that is optionally mono-, di-, or tri-substituted with 1, 2, or 3 halo. In more specific examples, BR₄ is benzo[d][1,3]dioxolyl that is optionally di-substituted with halo.

Another aspect of the present invention provides compounds of formula B1f:

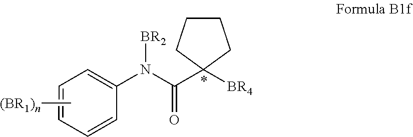

or a pharmaceutically acceptable salt thereof, wherein BR₁, BR₂, BR₄, and n are defined in Formula B.

Another aspect of the present invention provides compounds of formula BIg:

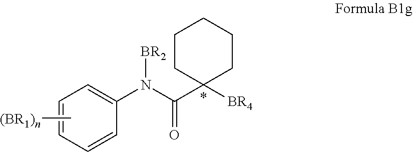

or a pharmaceutically acceptable salt thereof, wherein BR₁, BR₂, BR₄, and n are defined in Formula B.

Another aspect of the present invention provides compounds of Formula B1 h:

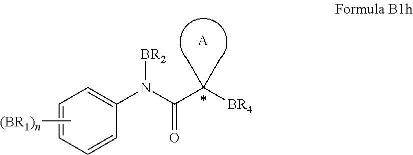

or a pharmaceutically acceptable salt thereof, wherein BR₁, BR₂, BR₄, and n are defined in Formula B.

Ring A is an optionally substituted heterocycloaliphatic.

In several embodiments, compounds of Formula B1 h include compounds of formulae B1h1:

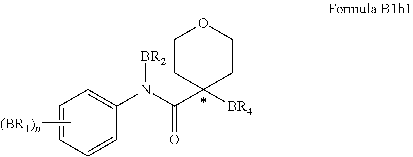

or a pharmaceutically acceptable salt thereof, wherein BR₁, BR₂, BR₄, and n are defined in Formula B.

Another aspect of the present invention provides compounds of formula B2:

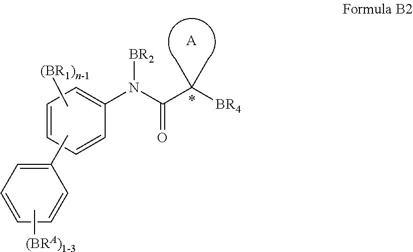

or a pharmaceutically acceptable salt thereof, wherein

BR₁, BR₂, ring A, and BR₄ are defined in Formula B;

n is 1, 2, 3, or 4; and

Each BRA is independently —ZABR5, wherein each ZA is independently a bond or an optionally substituted branched or straight C1-6 aliphatic chain wherein up to two carbon units of ZA are optionally and independently replaced by —CO—, —CS—, —CONBRB—, —CONBRBNBRB—, —CO2-, —OCO—, —NBRBCO2-, —O—, —NBRBCONBRB—, —OCONBRB—, —NBRBNBRB—, —NBRBCO—, —S—, —SO—, —SO2-, —NBRB—, —SO2NBRB—, —NBRBSO2-, or —NBRBSO2NBRB—. Each BR5 is independently BRB, halo, —OH, —NH2, —NO2, —CN, or —OCF3. Each BRB is independently hydrogen, an optionally substituted C1-8 aliphatic group, an optionally substituted cycloaliphatic, an optionally substituted heterocycloaliphatic, an optionally substituted aryl, or an optionally substituted heteroaryl.

In some embodiments, each BR₁ is an optionally substituted C1-6 aliphatic, an optionally substituted aryl, an optionally substituted heteroaryl, an optionally substituted 3 to 10 membered cycloaliphatic, or an optionally substituted 3 to 10 membered heterocycloaliphatic, each of which is optionally substituted with 1, 2, or 3 of BRA; wherein each BRA is —ZABR5, wherein each ZA is independently a bond or an optionally substituted branched or straight C1-6 aliphatic chain wherein up to two carbon units of ZA are optionally and independently replaced by —CO—, —CS—, —CONBRB—, —CONBRBNBRB—, —CO2-, —OCO—, —NBRBCO2-, —O—, —NBRBCONBRB—, —OCONBRB—, —NBRBNBRB—, —NBRBCO—, —S—, —SO—, —SO2-, —NBRB—, —SO2NBRB—, —NBRBSO2-, or —NBRBSO2NBRB—; and BR5 is independently BRB, halo, —OH, —NO2, —CN, or —OCF3; wherein each BRB is independently hydrogen, an optionally substituted C1-8 aliphatic group, an optionally substituted cycloaliphatic, an optionally substituted heterocycloaliphatic, an optionally substituted aryl, or an optionally substituted heteroaryl.

In some embodiments, BR₂ is C1-4 aliphatic, C3-4 cycloaliphatic, phenyl, or heteroaryl, each of which is optionally substituted, or BR₂ is hydrogen.

In some embodiments, ring A is an optionally substituted C3-7 cycloaliphatic or an optionally substituted C3-7 heterocycloaliphatic where the atoms of ring A adjacent to C* are carbon atoms, and said ring A is optionally substituted with 1, 2, or 3 of —ZBBR7, wherein each ZB is independently a bond, or an optionally substituted branched or straight C1-4 aliphatic chain wherein up to two carbon units of ZB are optionally and independently replaced by —CO—, —CS—, —CONBRB—, CONBRBNBRB—, —CO2-, —OCO—, —NBRBCO2-, —O—, —NBRBCONBRB—, —OCONBRB—, —NBRBNBRB—, —NBRBCO—, —S—, —SO—, —SO2-, —NBRB—, —SO2NBRB—, —NBRBSO2-, or —NBRBSO2NBRB—; Each BR7 is independently BRB, halo, —OH, —NH2, —NO2, —CN, or —OCF₃.

In some embodiments, each BR₄ is an aryl or heteroaryl, each of which is optionally substituted with 1, 2, or 3 of —ZCBR8, wherein each ZC is independently a bond or an optionally substituted branched or straight C1-6 aliphatic chain wherein up to two carbon units of ZC are optionally and independently replaced by —CO—, —CS—, —CONBRC—, —CONBRCNBRC—, —CO2-, —OCO—, —NBRCCO2-, —O—, —NBRCCONBRC—, —OCONBRC—, —NBRCNBRC—, —NBRCCO—, —S—, —SO—, —SO2-, —NBRC—, —SO2NBRC—, —NBRCSO2-, or —NBRCSO2NBRC—; wherein each BR8 is independently BRC, halo, —OH, —NH2, —NO2, —CN, or —OCF3; wherein each BRC is independently an optionally substituted C1-8 aliphatic group, an optionally substituted cycloaliphatic, an optionally substituted heterocycloaliphatic, an optionally substituted aryl, or an optionally substituted heteroaryl.

Another aspect of the present invention provides compounds of Formula B2a:

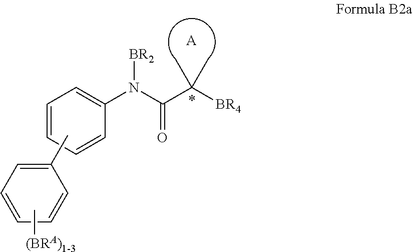

or pharmaceutically acceptable salts thereof, wherein BR₂, ring A and BR₄ are defined in Formula B, and BRA is defined above.

Another aspect of the present invention provides compounds of formula B2b:

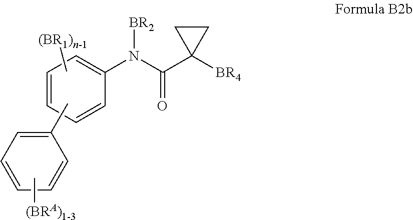

or a pharmaceutically acceptable salt thereof, wherein BR₁, BR₂, BR₄, and n are defined in Formula B and BRA is defined in Formula B2.

Another aspect of the present invention provides compounds of Formula B2c:

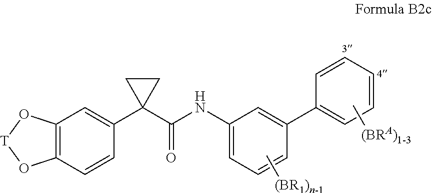

or a pharmaceutically acceptable salt thereof, wherein:

T is an optionally substituted C1-2 aliphatic chain, wherein each of the carbon units is optionally and independently replaced by —CO—, —CS—, —COCO—, —SO2-, —B(OH)—, or —B(O(C1-6 alkyl))-;

Each of BR₁ is independently an optionally substituted C1-6 aliphatic, an optionally substituted aryl, an optionally substituted heteroaryl, an optionally substituted 3 to 10 membered cycloaliphatic, an optionally substituted 3 to 10 membered heterocycloaliphatic, carboxy, amido, amino, halo, or hydroxy;

Each BRA is independently —ZABR5, wherein each ZA is independently a bond or an optionally substituted branched or straight C1-6 aliphatic chain wherein up to two carbon units of ZA are optionally and independently replaced by —CO—, —CS—, —CONBRB—, —CONBRBNBRB—, —CO2-, —OCO—, —NBRBCO2-, —O—, —NBRBCONBRB—, —OCONBRB—, —NBRBNBRB—, —NBRBCO—, —S—, —SO—, —SO2-, —NBRB—, —SO2NBRB—, —NBRBSO2-, or —NBRBSO2NBRB—;

Each BR5 is independently BRB, halo, —OH, —NH2, —NO2, —CN, —CF₃, or —OCF3; or two BRA, taken together with atoms to which they are attached, form a 3-8 membered saturated, partially unsaturated, or aromatic ring with up to 3 ring members independently selected from the group consisting of O, NH, NBRB, and S, provided that one BRA is attached to carbon 3″ or 4″.

Each BRB is independently hydrogen, an optionally substituted C1-8 aliphatic group, an optionally substituted cycloaliphatic, an optionally substituted heterocycloaliphatic, an optionally substituted aryl, or an optionally substituted heteroaryl.

n is 2 or 3 provided that when n is 3, a first BR₁ is attached ortho relative to the phenyl ring substituted with BRA and that a second one BR₁ is attached para relative to the phenyl ring substituted with BRA.

In some embodiments, T is an optionally substituted —CH2-. In some other embodiments, T is an optionally substituted —CH2CH2-.

In some embodiments, T is optionally substituted by —ZFBR10; wherein each ZF is independently a bond or an optionally substituted branched or straight C1-6 aliphatic chain wherein up to two carbon units of ZF are optionally and independently replaced by —CO—, —CS—, —CONBRF—, CONBRFNBRF—, —CO2-, —OCO—, —NBRFCO2-, —O—, —NBRFCONBRF—, —OCONBRF—, —NBRFNBRF—, —NBRFCO—, —S—, —SO—, —SO2-, —NBRF—, —SO2NBRF—, —NBRFSO2-, or —NBRFSO2NBRF—; R10 is independently RF, halo, —OH, —NH2, —NO2, —CN, —CF₃, or —OCF3; each BRF is independently hydrogen, an optionally substituted C1-8 aliphatic group, an optionally substituted cycloaliphatic, an optionally substituted heterocycloaliphatic, an optionally substituted aryl, or an optionally substituted heteroaryl. In one example, ZF is —O—.

In some embodiments, BR10 is an optionally substituted C1-6 alkyl, an optionally substituted C2-6 alkenyl, an optionally substituted C3-7 cycloaliphatic, or an optionally substituted C6-10 aryl. In one embodiment, BR10 is methyl, ethyl, iso-propyl, or tert-butyl.

In some embodiments, up to two carbon units of T are independently and optionally replaced with —CO—, —CS—, —B(OH)—, or —B(O(C1-6 alkyl)-.

In some embodiments, T is selected from the group consisting of —CH2-, —CH2CH2-, —CF2-, —C(CH3)2-, —C(O)—,

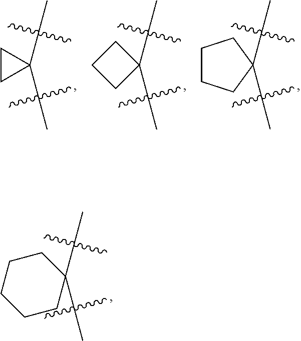 [SMILES: *C1(*)CC1 ; *C1(*)CCC1 ; *C1(*)CCCC1 ; *C1(*)CCCCC1]

—C(phenyl)₂-, —B(OH)—, and —CH(OEt)-. In some embodiments, T is —CH2-, —CF2-, —C(CH3)2-,

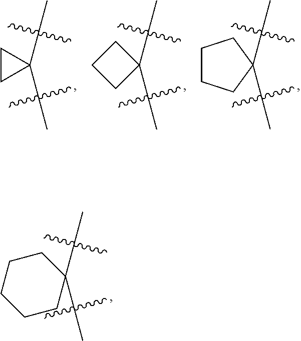 [SMILES: *C1(*)CC1 ; *C1(*)CCC1 ; *C1(*)CCCC1 ; *C1(*)CCCCC1]

or —C(Phenyl)₂-. In other embodiments, T is —CH2H2-, —C(O)—, —B(OH)—, and —CH(OEt)-. In several embodiments, T is —CH2-, —CF2-, —C(CH3)2-,

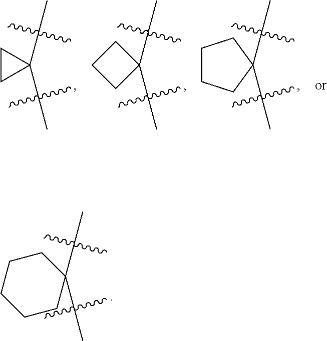 [SMILES: *C1(*)CC1 ; *C1(*)CCC1 ; *C1(*)CCCC1 ; *C1(*)CCCCC1]

More preferably, T is —CH2-, —CF2-, or —C(CH3)2-. In several embodiments, T is —CH2-. Or, T is —CF₂—. Or, T is —C(CH3)2-. Or, T is

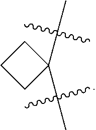 [SMILES: *C1(*)CCC1]

In some embodiments, each BR₁ is hydrogen. In some embodiments, each of BR₁ is independently —ZEBR9, wherein each ZE is independently a bond or an optionally substituted branched or straight C1-6 aliphatic chain wherein up to two carbon units of ZE are optionally and independently replaced by —CO—, —CS—, —CONBRE-, —CONBRENBRE-, —CO2-, —OCO—, —NBRECO2-, —O—, —NBRECONBRE-, —OCONBRE-, —NBRENBRE-, —NBRECO—, —S—, —SO—, —SO2-, —NBRE-, —SO2NBRE-, —NBRESO2-, or —NBRESO2NBRE-. Each BR9 is independently H, BRE, halo, —OH, —NH2, —NO2, —CN, —CF₃, or —OCF3. Each BRE is independently an optionally substituted group selected from C1-8 aliphatic group, cycloaliphatic, heterocycloaliphatic, aryl, and heteroaryl.

In several embodiments, a first BR₁ is attached ortho relative to the phenyl ring substituted with BRA is —H, —F, —Cl, —CF₃, —OCH3, —OCF3, methyl, ethyl, iso-propyl, or tert-butyl.

In several embodiments, a first BR₁ is attached ortho relative to the phenyl ring substituted with RA is —ZEBR9, wherein each ZE is independently a bond or an optionally substituted branched or straight C1-6 aliphatic chain wherein up to two carbon units of ZE are optionally and independently replaced by —CO—, —CONBRE-, —CO2-, —O—, —S—, —SO—, —SO2-, —NBRE-, or —SO2NBRE-. Each BR9 is hydrogen, BRE, halo, —OH, —NH2, —CN, —CF3, or —OCF3. Each BRE is independently an optionally substituted group selected from the group including C1-8 aliphatic group, a cycloaliphatic, a heterocycloaliphatic, an aryl, and a heteroaryl. In one embodiment, ZE is a bond. In one embodiment, ZE is a straight C1-6 aliphatic chain, wherein one carbon unit of ZE is optionally replaced by —CO—, —CONBRE-, —CO2-, —O—, or —NBRE-. In one embodiment, ZE is a C1-6 alkyl chain. In one embodiment, ZE is —CH2-. In one embodiment, ZE is —CO—. In one embodiment, ZE is —CO2-. In one embodiment, ZE is —CONRE-. In one embodiment, ZE is —CO—.

In some embodiments, BR9 is H, —NH2, hydroxy, —CN, or an optionally substituted group selected from the group of C1-8 aliphatic, C3-8 cycloaliphatic, 3-8 membered heterocycloaliphatic, C6-10 aryl, and 5-10 membered heteroaryl. In one embodiment, BR9 is H. In one embodiment, BR9 is hydroxy. Or, BR9 is —NH2. Or, BR9 is —CN. In some embodiments, BR9 is an optionally substituted 3-8 membered heterocycloaliphatic, having 1, 2, or 3 ring members independently selected from nitrogen (including NH and NBRX), oxygen, and sulfur (including S, SO, and SO2). In one embodiment, BR9 is an optionally substituted five membered heterocycloaliphatic with one nitrogen (including NH and NBRX) ring member.

In one embodiment, BR9 is an optionally substituted pyrrolidin-1-yl. Examples of said optionally substituted pyrrolidin-1-yl include pyrrolidin-1-yl and 3-hydroxy-pyrrolidin-1-yl. In one embodiment, BR9 is an optionally substituted six membered heterocycloaliphatic with two heteroatoms independently selected from nitrogen (including NH and NBRX) and oxygen. In one embodiment, BR9 is morpholin-4-yl. In some embodiments, BR9 is an optionally substituted 5-10 membered heteroaryl. In one embodiment, BR9 is an optionally substituted 5 membered heteroaryl, having 1, 2, 3, or 4 ring members independently selected from nitrogen (including NH and NBRX), oxygen, and sulfur (including S, SO, and SO2). In one embodiment, BR₉ is 1H-tetrazol-5-yl.

In one embodiment, a first BR₁ is attached ortho relative to the phenyl ring substituted with BRA is ZEBR9; wherein ZE is CH2 and BR9 is 1H-tetrazol-5-yl. In one embodiment, one BR1′ is ZEBR9; wherein ZE is CH2 and BR9 is morpholin-4-yl. In one embodiment, one BR1′ is ZEBR9; wherein ZE is CH2 and BR9 is pyrrolidin-1-yl. In one embodiment, one BR1′ is ZEBR9; wherein ZE is CH2 and BR9 is 3-hydroxy-pyrrolidin-1-yl. In one embodiment, one BR₁′ is ZEBR9; wherein ZE is C0 and BR9 is 3-hydroxy-pyrrolidin-1-yl.

In some embodiments, a first BR₁ is attached ortho relative to the phenyl ring substituted with BRA is selected from CH2OH, COOH, CH₂OCH3, COOCH3, CH2NH2, CH2NHCH3, CH2CN, CONHCH3, CH2CONH2, CH2OCH2CH3, CH2N(CH3)2, CON(CH3)2, CH2NHCH2CH2OH, CH2NHCH2CH2COOH, CH2OCH(CH3)2, CONHCH(CH3)CH2OH, or CONHCH(tert-butyl)CH2OH.

In some embodiments, a first BR₁ is attached ortho relative to the phenyl ring substituted with BRA is an optionally substituted C3-10 cycloaliphatic or an optionally substituted 4-10 membered heterocycloaliphatic. In one embodiment, BR1′ is an optionally substituted 4, 5, or 6 membered heterocycloallyl containing one oxygen atom. In one embodiment, BR1′ is 3-methyloxetan-3-yl.

In some embodiments, a second one BR₁ is attached para relative to the phenyl ring substituted with BRA is selected from the group consisting of H, halo, optionally substituted C1-6 aliphatic, and optionally substituted —O(C1-6 aliphatic). In some embodiments, a second one BR₁ is attached para relative to the phenyl ring substituted with BRA is selected from the group consisting of H, methyl, ethyl, iso-propyl, tert-butyl, F, Cl, CF3, —OCH3, —OCH2CH3, —O-(iso-propyl), —O-(tert-butyl), and —OCF3. In one embodiment, a second one BR₁ is attached para relative to the phenyl ring substituted with RA is H. In one embodiment, a second one BR₁ is attached para relative to the phenyl ring substituted with BRA is methyl. In one embodiment, a second one BR₁ is attached para relative to the phenyl ring substituted with BRA is F. In one embodiment, a second one BR₁ is attached para relative to the phenyl ring substituted with BRA is —OCF3. In one embodiment, a second one BR₁ is attached para relative to the phenyl ring substituted with BRA is —OCH3.

In some embodiments, one BRA is attached to carbon 3″ or 4″ and is —ZABR5, wherein each ZA is independently a bond or an optionally substituted branched or straight C1-6 aliphatic chain wherein up to two carbon units of ZA are optionally and independently replaced by —CO—, —CS—, —CONBRB—, —CONBRBNBRB—, —CO2-, —OCO—, —NBRBCO2-, —O—, —NBRBCONBRB—, —OCONBRB—, —NBRBNBRB—, —NBRBCO—, —S—, —SO—, —SO2-, —NBRB—, —SO2NBRB—, —NBRBSO2-, or —NBRBSO2NBRB—. In yet some embodiments, ZA is independently a bond or an optionally substituted branched or straight C1-6 aliphatic chain wherein one carbon unit of ZA is optionally replaced by —CO—, —SO—, —SO2-, —COO—, —OCO—, —CONBRB—, —NBRBCO—, —NBRBCO2-, —O—, —NBRBSO2-, or —SO2NBRB—. In some embodiments, one carbon unit of ZA is optionally replaced by —CO—. Or, by —SO—. Or, by —SO2-. Or, by —COO—. Or, by —OCO—. Or, by —CONBRB—. Or, by —NBRBCO—. Or, by —NBRBCO2-. Or, by —O—. Or, by —NBRBSO2-. Or, by —SO2NBRB—.

In several embodiments, BR5 is hydrogen, halo, —OH, —NH2, —CN, —CF3, —OCF3, or an optionally substituted group selected from the group consisting of C1-6 aliphatic, C3-8 cycloaliphatic, 3-8 membered heterocycloaliphatic, C6-10 aryl, and 5-10 membered heteroaryl. In several examples, BR5 is hydrogen, F, Cl, —OH, —CN, —CF3, or —OCF3. In some embodiments, BR5 is C1-6 aliphatic, C3-8 cycloaliphatic, 3-8 membered heterocycloaliphatic, C6-10 aryl, and 5-10 membered heteroaryl, each of which is optionally substituted with 1 or 2 substituents independently selected from the group consisting of BRB, oxo, halo, —OH, —NBRBBRB, —OBRB, —COOBRB, and —CONBRBBRB. In several examples, BR5 is optionally substituted by 1 or 2 substituents independently selected from the group consisting of oxo, F, Cl, methyl, ethyl, iso-propyl, tert-butyl, —CH2OH, —CH2CH2OH, —C(O)OH, —C(O)NH2, —CH2O(C1-6 alkyl), —CH2CH2O(C1-6 alkyl), and —C(O)(C1-6 alkyl).

In one embodiment, BR5 is hydrogen. In some embodiments, BR5 is selected from the group consisting of straight or branched C1-6 alkyl or straight or branched C2-6 alkenyl; wherein said alkyl or alkenyl is optionally substituted with 1 or 2 substituents independently selected from the group consisting of RB, oxo, halo, —OH, —NBRBBRB, —OBRB, —COOBRB, and —CONBRBBRB.

In other embodiments, BR5 is C3-8 cycloaliphatic optionally substituted with 1 or 2 substituents independently selected from the group consisting of BRB, oxo, halo, —OH, —NBRBBRB, —OBRB, —COOBRB, and —CONBRBBRB. Examples of cycloaliphatic include but are not limited to cyclopropyl, cyclobutyl, cyclopentyl, cyclohexyl, and cycloheptyl.

In yet other embodiments, BR5 is a 3-8 membered heterocyclic with 1 or 2 heteroatoms independently selected from the group consisting of nitrogen (including NH and NBRX), oxygen, and sulfur (including S, SO, and SO2); wherein said heterocyclic is optionally substituted with 1 or 2 substituents independently selected from the group BRB, oxo, halo, —OH, —NBRBBRB, —OBRB, —COOBRB, and —CONBRBBRB. Examples of 3-8 membered heterocyclic include but are not limited to

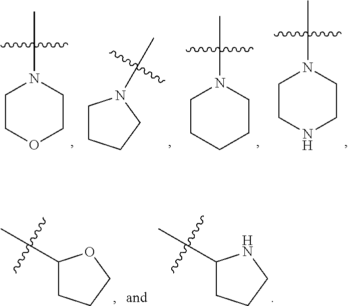

In yet some other embodiments, BR5 is an optionally substituted 5-8 membered heteroaryl with one or two ring atom independently selected from the group consisting of nitrogen (including NH and NRX), oxygen, and sulfur (including S, SO, and SO2). Examples of 5-8 membered heteroaryl include but are not limited to

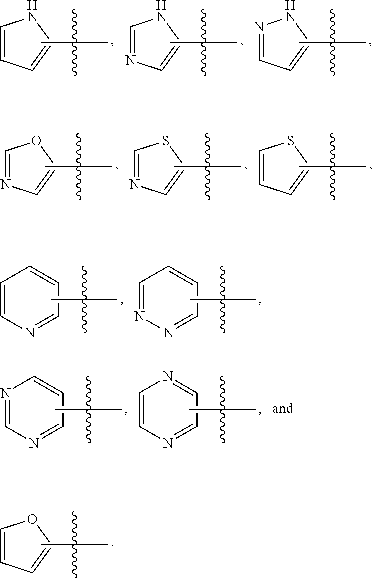

In some embodiments, two BRAs, taken together with carbons to which they are attached, form an optionally substituted 4-8 membered saturated, partially unsaturated, or aromatic ring with 0-2 ring atoms independently selected from the group consisting of nitrogen (including NH and NBRX), oxygen, and sulfur (including S, SO, and SO2). Examples of two BRAs, taken together with phenyl containing carbon atoms to which they are attached, include but are not limited to

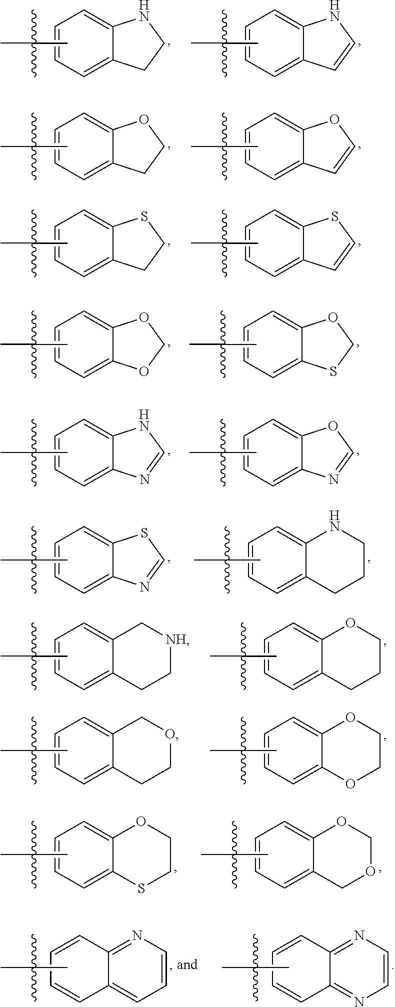

In some embodiments, one BRA not attached top the carbon 3″ or 4″ is selected from the group consisting of H, BRB, halo, —OH, —(CH2)rNBRBBRB, —(CH2)r—OBRB, —SO2-BRB, —NBRB—SO2-BRB, —SO2NBRBBRB, —C(O)BRB, —C(O)OBRB, —OC(O)OBRB, —NBRBC(O)OBRB, and —C(O)NBRBBRB; wherein r is 0, 1, or 2; and each BRB is independently hydrogen, an optionally substituted C1-8 aliphatic group, an optionally substituted cycloaliphatic, an optionally substituted heterocycloaliphatic, an optionally substituted aryl, or an optionally substituted heteroaryl. In other embodiments, one BRA not attached top the carbon 3″ or 4″ is selected from the group consisting of H, C1-6 aliphatic, halo, —CN, —NH2, —NH(C1-6 aliphatic), —N(C1-6 aliphatic)2, —CH2-N(C1-6 aliphatic)2, —CH2-NH(C1-6 aliphatic), —CH2NH2, —OH, —O(C1-6 aliphatic), —CH2OH, —CH2-O(C1-6 aliphatic), —SO2(C1-6 aliphatic), —N(C1-6 aliphatic)-SO2(C1-6 aliphatic), —NH—SO2(C1-6 aliphatic), —SO2NH2, —SO2NH(C1-6 aliphatic), —SO2N(C1-6 aliphatic)2, —C(O)(C1-6 aliphatic), —C(O)O(C1-6 aliphatic), —C(O)OH, —OC(O)O(C1-6 aliphatic), —NHC(O)(C1-6 aliphatic), —NHC(O)O(C1-6 aliphatic), —N(C1-6 aliphatic)C(O)O(C1-6 aliphatic), —C(O)NH2, and —C(O)N(C1-6 aliphatic)₂. In several examples, BRA2 is selected from the group consisting of H, C1-6 aliphatic, halo, —CN, —NH2, —CH2NH2, OH, —O(C1-6 aliphatic), —CH2OH, —SO2(C1-6 aliphatic), —NH—SO2(C1-6 aliphatic), —C(O)O(C1-6 aliphatic), —C(O)OH, —NHC(O)(C1-6 aliphatic), —C(O)NH2, —C(O)NH(C1-6 aliphatic), and —C(O)N(C1-6 aliphatic)2. For examples, one BRA not attached top the carbon 3″ or 4″ is selected from the group consisting of H, methyl, ethyl, n-propyl, iso-propyl, tert-butyl, F, Cl, CN, —NH2, —CH2NH2, —OH, —OCH3, —O-ethyl, —O-(iso-propyl), —O-(n-propyl), —CH2OH, —SO2CH3, —NH—SO2CH3, —C(O)OCH3, —C(O)OCH2CH3, —C(O)OH, NHC(O)CH3, —C(O)NH2, and —C(O)N(CH3)2. In one embodiment, all BRAs not attached top the carbon 3″ or 4″ are hydrogen. In another embodiment, one BRA not attached top the carbon 3″ or 4″ is methyl. Or, one BRA not attached top the carbon 3″ or 4″ is ethyl. Or, one BRA not attached top the carbon 3″ or 4″ is F. Or, one BRA not attached top the carbon 3″ or 4″ is Cl. Or, one BRA not attached top the carbon 3″ or 4″ is —OCH3.

In one embodiment, the present invention provides compounds of Formula B2d or Formula B2e:

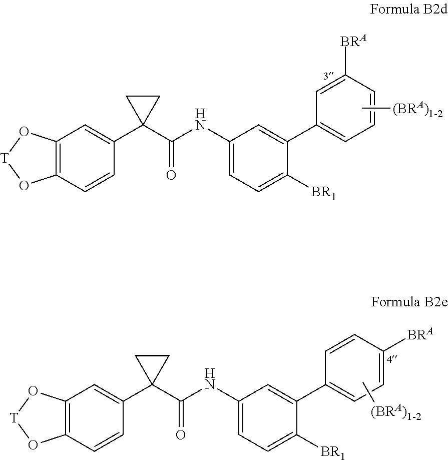

wherein T, each BRA, and BR₁ are as defined above.

In one embodiment, T is —CH2-, —CF₂—, —C(CH3)₂—, or

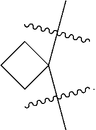 [SMILES: *C1(*)CCC1]

In one embodiment, T is —CH2-. In one embodiment, T is —CF₂—. In one embodiment, T is —C(CH3)₂—. In one embodiment, T is

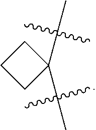 [SMILES: *C1(*)CCC1]

In one embodiment, BR₁ is selected from the group consisting of H, halo, —CF3, or an optionally substituted group selected from —C1-6 aliphatic, —O(C1-6 aliphatic), —C3-5 cycloalkyl, 3-6 membered heterocycloalkyl containing one oxygen atom, carboxy, and aminocarbonyl. Said —C1-6 aliphatic, —O(C1-6 aliphatic), —C3-5 cycloalkyl, 3-6 membered heterocycloalkyl containing one oxygen atom, carboxy, or aminocarbonyl is optionally substituted with halo, —CN, hydroxy, or a group selected from amino, branched or straight C1-6 aliphatic, branched or straight alkoxy, aminocarbonyl, C3-8 cycloaliphatic, 3-10 membered heterocyclicaliphatic having 1, 2, or 3 ring membered independently selected from nitrogen (including NH and NBRX), oxygen, or sulfur (including S, SO, and SO2), C6-10 aryl, and 5-10 membered heteroaryl, each of which is further optionally substituted with halo or hydroxy. Exemplary embodiments include H, methyl, ethyl, iso-propyl, tert-butyl, F, Cl, CF3, CHF2, —OCF3, —OCH3, —OCH2CH3, —O-(iso-propyl), —O-(tert-butyl), —COOH, —COOCH3, —CONHCH(tert-butyl)CH2OH, —CONHCH(CH3)CH2OH, —CON(CH3)2, —CONHCH3, —CH2CONH2, pyrrolid-1-yl-methyl, 3-hydroxy-pyrrolid-1-yl-methyl, morpholin-4-yl-methyl, 3-hydroxy-pyrrolid-1-yl-formyl, tetrazol-5-yl-methyl, cyclopropyl, hydroxymethyl, methoxymethyl, ethoxymethyl, methylaminomethyl, dimethylaminomethyl, cyanomethyl, 2-hydroxyethylaminomethyl, iso-propoxymethyl, or 3-methyloxetan-3-yl. In still other embodiments, BR₁ is H. Or, BR₁ is methyl. Or, BR₁ is ethyl. Or, BR₁ is CF3. Or, BR₁ is oxetanyl.

In some embodiments, BRA attached at the carbon carbon 3″ or 4″ is H, halo, —OH, —CF3, —OCF3, —CN, —SCH3, or an optionally substituted group selected from C1-6 aliphatic, amino, alkoxy, or 3-8 membered heterocycloaliphatic having 1, 2, or 3 ring members each independently chosen from nitrogen (including NH and NBRX), oxygen, or sulfur (including S, SO, and SO2). In some embodiments, BRA attached at the carbon carbon 3″ or 4″ is H, F, Cl, OH, CF3, OCF3, CN, or SCH3. In some embodiments, BRA attached at the carbon carbon 3″ or 4″ is C1-6 alkyl, amino, alkoxy, or 3-8 membered heterocycloalkyl having 1, 2, or 3 ring members each independently chosen from nitrogen (including NH and NBRX), oxygen, or sulfur (including S, SO, and SO2); wherein said alkyl, amino, alkoxy, or heterocycloalkyl each is optionally substituted with 1, 2, or 3 groups independently selected from oxo, halo, hydroxy, or an optionally substituted group selected from C1-6 aliphatic, cycloaliphatic, heterocycloaliphatic, aryl, heteroaryl, carbonyl, amino, and carboxy. In one embodiment, BRA attached at the carbon carbon 3″ or 4″ is H, F, Cl, —OH, —CF3, —OCF3, —CN, —SCH3, methyl, ethyl, iso-propyl, tert-butyl, 2-methylpropyl, cyanomethyl, aminomethyl, hydroxymethyl, 1-hydroxyethyl, methoxymethyl, methylaminomethyl, (2′-methylpropylamino)-methyl, 1-methyl-1-cyanoethyl, n-propylaminomethyl, dimethylaminomethyl, 2-(methylsulfonyl)-ethyl, CH2COOH, CH(OH)COOH, diethylamino, piperid-1-yl, 3-methyloxetan-3-yl, 2,5-dioxopyrrolid-1-yl, morpholin-4-yl, 2-oxopyrrolid-1-yl, tetrazol-5-yl, methoxy, ethoxy, OCH2COOH, amino, dimethylamino, NHCH2COOH, or acetyl.

In one embodiment, BRA attached at the carbon carbon 3″ or 4″ is ZABR5, wherein ZA is selected from —CONH—, —CON(C1-6 alkyl)-, NHCO—, SO2NH, SO2N(C1-6 alkyl)-, NHSO2-, —CH2NHSO2-, CH2N(CH3)SO2-, —CH2NHCO—, —CH2N(CH3)CO—, —COO—, —SO2-, —SO—, or —CO—. In one embodiment, BRA attached at the carbon carbon 3″ or 4″ is ZABR5, wherein ZA is selected from —CONH—, —SO2NH—, —SO2N(C1-6 alkyl)-, —CH2NHSO2-, —CH2N(CH3)SO2-, —CH2NHCO—, —COO—, —SO2-, or —CO—.

In one embodiment, ZA is COO and BR5 is H. In one embodiment, ZA is COO and BR5 is an optionally substituted straight or branched C1-6 aliphatic. In one embodiment, ZA is COO and BR5 is an optionally substituted straight or branched C1-6 alkyl. In one embodiment, ZA is COO and BR5 is C1-6 alkyl. In one embodiment, ZA is COO and BR5 is methyl.

In one embodiment, ZA is CONH and BR5 is H. In one embodiment, ZA is CONH and BR5 is an optionally substituted straight or branched C1-6 aliphatic. In one embodiment, ZA is CONH and BR5 is C1-6 straight or branched alkyl optionally substituted with one or more groups independently selected from —OH, halo, CN, optionally substituted C1-6 alkyl, optionally substituted C3-10 cycloaliphatic, optionally substituted 3-8 membered heterocycloaliphatic, optionally substituted C6-10 aryl, optionally substituted 5-8 membered heteroaryl, optionally substituted alkoxy, optionally substituted amino, and optionally substituted aminocarbonyl. In one embodiment, ZA is CONH and BR5 is 2-(dimethylamino)ethyl, cyclopropylmethyl, cyclohexylmethyl, 2-(cyclohexen-1-yl)ethyl, 3-(morpholin-4-yl)propyl, 2-(morpholin-4-yl)ethyl, 2-(1H-imidazol-4-yl)ethyl, tetrahydrofuran-2-yl-methyl, 2-(pyrid-2-yl)ethyl, (1-ethyl-pyrrolidin-2-yl)methyl, 1-hydroxymethylpropyl, 1-hydroxymethylbutyl, 1-hydroxymethylpentyl, 1-hydroxymethyl-2-hydroxyethyl, 1-hydroxymethyl-2-methylpropyl, 1-hydroxymethyl-3-methyl-butyl, 2,2-dimethyl-1-hydroxymethyl-propyl, 1,1-di(hydroxymethyl)ethyl, 1,1-di(hydroxymethyl)propyl, 3-ethoxypropyl, 2-acetoaminoethyl, 2-(2′-hydroxyethoxy)ethyl, 2 hydroxyethyl, 3-hydroxypropyl, 2-hydroxypropyl, 4-hydroxybutyl, 2,3-dihydroxypropyl, 2-hydroxy-1-methylethyl, 2-methoxyethyl, 3-methoxypropyl, 2-cyanoethyl, or aminoformylmethyl. In one embodiment, ZA is CONH and BR5 is straight or branched C1-6 alkyl. In one embodiment, ZA is CONH and R5 is methyl, ethyl, n-propyl, iso-propyl, 3-methylbutyl, 3,3-dimethylbutyl, 2-methylpropyl, or tert-butyl.

In one embodiment, ZA is CONH and BR5 is an optionally substituted C3-10 cycloaliphatic. In one embodiment, ZA is CONH and BR5 is an optionally substituted C3-10 cycloalkyl. In one embodiment, ZA is CONH and BR5 is cyclopropyl, cyclobutyl, cyclopentyl, or cyclohexyl.

In some embodiments, ZA is CONH and BR5 is an optionally substituted 3-8 membered heterocycloaliphatic. In several examples, ZA is CONH and BR5 is an optionally substituted 3-8 membered heterocycloalkyl, having 1, 2, or 3 ring members independently selected from nitrogen (including NH and NBRX), oxygen, or sulfur (including S, SO, and SO2). In several examples, ZA is CONH and BR5 is 3-8 membered heterocycloalkyl optionally substituted with 1, 2, or 3 groups independently selected from oxo, halo, hydroxy, or an optionally substituted group selected from C1-6 aliphatic, cycloaliphatic, heterocycloaliphatic, aryl, heteroaryl, carbonyl, amino, and carboxy. In one embodiment, ZA is CONH and BR5 is 3-oxo-isoxazolidin-4-yl.

In some embodiments, ZA is CON(C1-6 aliphatic) and BR5 is an optionally substituted C1-6 aliphatic or an optionally substituted C3-8 cycloaliphatic. In some embodiments, ZA is CON(branched or straight C1-6 alkyl) and BR5 is branched or straight C1-6 alkyl or C3-8 cycloaliphatic, each optionally substituted with 1, 2, or 3 groups independently selected from CN, OH, and an optionally substituted group chosen from amino, branched or straight C1-6 aliphatic, C3-8 cycloaliphatic, 3-8 membered heterocycloaliphatic, C6-10 aryl, and 5-10 membered heteroaryl. In one embodiment, ZA is CON(CH3) and BR5 is methyl, ethyl, n-propyl, butyl, 2-pyrid-2-ylethyl, dimethylaminomethyl, 2-dimethylaminoethyl, 1,3-dioxolan-2-ylmethyl, 2-cyanoethyl, cyanomethyl, or 2-hydroxyethyl. In one embodiment, ZA is CON(CH2CH3) and BR5 is ethyl, propyl, iso-propyl, n-butyl, tert-butyl, cyclohexyl, 2-dimethylaminoethyl, or 2-hydroxyethyl. In one embodiment, ZA is CON(CH2CH2CH3) and BR5 is cyclopropylmethyl or 2-hydroxyethyl. In one embodiment, ZA is CON(iso-propyl) and BR5 is iso-propyl.

In some embodiments, ZA is CH2NHCO and BR5 is an optionally substituted straight or branched C1-6 aliphatic, an optionally substituted C3-8 cycloaliphatic, an optionally substituted alkoxy, or an optionally substituted heteroaryl. In some embodiments, ZA is CH2NHCO and BR5 is straight or branched C1-6 alkyl, C3-8 cycloalkyl, or alkoxy, each of which is optionally substituted with 1, 2, or 3 groups independently selected from halo, oxo, hydroxy, or an optionally substituted group selected from C1-6 aliphatic, C3-8 cycloaliphatic, 3-8 membered heterocycloaliphatic, C6-10 aryl, 5-10 membered heteroaryl, alkoxy, amino, carboxyl, and carbonyl. In one embodiment, ZA is CH2NHCO and BR5 is methyl, ethyl, 1-ethylpropyl, 2-methylpropyl, 1-methylpropyl, 2,2-dimethylpropyl, n-propyl, iso-propyl, n-butyl, tert-butyl, cyclopentyl, dimethylaminomethyl, methoxymethyl, (2′-methoxyethoxy)methyl, (2′-methoxy)ethoxy, methoxy, ethoxy, iso-propoxy, or tert-butoxy. In one embodiment, ZA is CH2NHCO and BR5 is an optionally substituted heteroaryl. In one embodiment, ZA is CH2NHCO and BR5 is pyrazinyl.

In some embodiments, ZA is CH2N(CH3)CO and BR5 is an optionally substituted straight or branched C1-6 aliphatic, C3-8 cycloaliphatic, or an optionally substituted heteroaryl. In some embodiments, ZA is CH2N(CH3)CO and BR5 is straight or branched C1-6 alkyl, or 5 or 6 membered heteroaryl, each of which is optionally substituted with 1, 2, or 3 groups independently selected from halo, oxo, hydroxy, or an optionally substituted group selected from C1-6 aliphatic, C3-8 cycloaliphatic, 3-8 membered heterocycloaliphatic, C6-10 aryl, 5-10 membered heteroaryl, alkoxy, amino, carboxyl, and carbonyl. In one embodiment, ZA is CH2N(CH3)CO and BR5 is methoxymethyl, (2′-methoxyethoxy)methyl, dimethylaminomethyl, or pyrazinyl. In some embodiments, ZA is CH2N(CH3)CO and BR5 is branched or straight C1-6 alkyl or C3-8 cycloalkyl. In one embodiment, ZA is CH2N(CH3)CO and BR5 is methyl, ethyl, iso-propyl, n-propyl, n-butyl, tert-butyl, 1-ethylpropyl, 2-methylpropyl, 2,2-dimethylpropyl, or cyclopentyl.

In one embodiment, ZA is SO2NH and BR5 is H. In some embodiments, ZA is SO2NH and BR5 is an optionally substituted straight or branched C1-6 aliphatic. In some embodiments, ZA is SO2NH and BR5 is straight or branched C1-6 alkyl optionally substituted with halo, oxo, hydroxy, or an optionally substituted group selected from C1-6 aliphatic, C3-8 cycloaliphatic, 3-8 membered heterocycloaliphatic, C6-10 aryl, 5-10 membered heteroaryl, alkoxy, amino, amido, carboxyl, or carbonyl. In one embodiment, ZA is SO2NH and BR5 is methyl. In one embodiment, ZA is SO2NH and BR5 is ethyl. In one embodiment, ZA is SO2NH and BR5 is n-propyl. In one embodiment, ZA is SO2NH and BR5 is iso-propyl. In one embodiment, ZA is SO2NH and BR5 is tert-butyl. In one embodiment, ZA is SO2NH and BR5 is 3,3-dimethylbutyl. In one embodiment, ZA is SO2NH and BR5 is CH2CH2OH. In one embodiment, ZA is SO2NH and BR5 is CH2CH2OCH3. In one embodiment, ZA is SO2NH and BR5 is CH(CH3)CH2OH. In one embodiment, ZA is SO2NH and BR5 is CH2CH(CH3)OH. In one embodiment, ZA is SO2NH and BR5 is CH(CH2OH)2. In one embodiment, ZA is SO2NH and BR5 is CH2CH(OH)CH2OH. In one embodiment, ZA is SO2NH and BR5 is CH2CH(OH)CH2CH3. In one embodiment, ZA is SO2NH and BR5 is C(CH3)₂CH2OH. In one embodiment, ZA is SO2NH and BR5 is CH(CH2CH3)CH2OH. In one embodiment, ZA is SO2NH and BR5 is CH2CH2OCH2CH2OH. In one embodiment, ZA is SO2NH and BR5 is C(CH3)(CH2OH)2. In one embodiment, ZA is SO2NH and BR5 is CH(CH3)C(O)OH. In one embodiment, ZA is SO2NH and BR5 is CH(CH2OH)C(O)OH. In one embodiment, ZA is SO2NH and BR5 is CH2C(O)OH. In one embodiment, ZA is SO2NH and BR5 is CH2CH2C(O)OH. In one embodiment, ZA is SO2NH and BR5 is CH2CH(OH)CH2C(O)OH. In one embodiment, ZA is SO2NH and BR5 is CH2CH2N(CH3)2. In one embodiment, ZA is SO2NH and BR5 is CH2CH2NHC(O)CH3. In one embodiment, ZA is SO2NH and BR5 is CH(CH(CH3)2)CH2OH. In one embodiment, ZA is SO2NH and BR5 is CH(CH2CH2CH3)CH2OH. In one embodiment, ZA is SO2NH and BR5 is tetrahydrofuran-2-ylmethyl. In one embodiment, ZA is SO2NH and BR5 is furylmethyl. In one embodiment, ZA is SO2NH and BR5 is (5-methylfuryl)-methyl. In one embodiment, ZA is SO2NH and BR5 is 2-pyrrolidinylethyl. In one embodiment, ZA is SO2NH and BR5 is 2-(1-methylpyrrolidinyl)-ethyl. In one embodiment, ZA is SO2NH and BR5 is 2-(morpholin-4-yl)-ethyl. In one embodiment, ZA is SO2NH and BR5 is 3-(morpholin-4-yl)-propyl. In one embodiment, ZA is SO2NH and BR5 is C(CH2CH3)(CH2OH)2. In one embodiment, ZA is SO2NH and BR5 is 2-(1H-imidazol-4-yl) ethyl. In one embodiment, ZA is SO2NH and BR5 is 3-(1H-imidazol-1-yl)-propyl. In one embodiment, ZA is SO2NH and BR5 is 2-(pyridin-2-yl)-ethyl.

In some embodiment, ZA is SO2NH and BR5 is an optionally substituted C3-8 cycloaliphatic. In several examples, ZA is SO2NH and BR5 is an optionally substituted C3-8 cycloalkyl. In several examples, ZA is SO2NH and BR5 is C3-8 cycloalkyl. In one embodiment, ZA is SO2NH and BR5 is cyclobutyl. In one embodiment, ZA is SO2NH and BR5 is cyclopentyl. In one embodiment, ZA is SO2NH and BR5 is cyclohexyl.

In some embodiment, ZA is SO2NH and BR5 is an optionally substituted 3-8 membered heterocycloaliphatic. In several examples, ZA is SO2NH and BR5 is an optionally substituted 3-8 membered heterocycloalkyl, having 1, 2, or 3 ring members independently selected from nitrogen (including NH and NBRX), oxygen, or sulfur (including S, SO, and SO2). In several examples, ZA is SO2NH and BR5 is 3-8 membered heterocycloalkyl optionally substituted with 1, 2, or 3 groups independently selected from oxo, halo, hydroxy, or an optionally substituted group selected from C1-6 aliphatic, aryl, heteroaryl, carbonyl, amino, and carboxy. In one embodiment, ZA is SO2NH and BR5 is 3-oxo-isoxazolidin-4-yl.

In some embodiments, ZA is SO2N(C1-6 alkyl) and BR5 is an optionally substituted straight or branched C1-6 aliphatic or an optionally substituted cycloaliphatic. In some embodiments, ZA is SO2N(C1-6 alkyl) and BR5 is an optionally substituted straight or branched C1-6 aliphatic. In some embodiments, ZA is SO2N(C1-6 alkyl) and BR5 is an optionally substituted straight or branched C1-6 alkyl or an optionally substituted straight or branched C2-6 alkenyl. In one embodiments, ZA is SO2N(CH3) and BR5 is methyl. In one embodiments, ZA is SO2N(CH3) and BR5 is n-propyl. In one embodiments, ZA is SO2N(CH3) and BR5 is n-butyl. In one embodiments, ZA is SO2N(CH3) and BR5 is cyclohexyl. In one embodiments, ZA is SO2N(CH3) and BR5 is allyl. In one embodiments, ZA is SO2N(CH3) and BR5 is CH2CH2OH. In one embodiments, ZA is SO2N(CH3) and BR5 is CH2CH(OH)CH2OH. In one embodiments, ZA is SO2N(ethyl) and BR5 is ethyl. In one embodiment, ZA is SO2N(CH2CH3) and BR5 is CH2CH3OH. In one embodiments, ZA is SO2N(CH2CH2CH3) and BR5 is cyclopropylmethyl. In one embodiments, ZA is SO2N(n-propyl) and BR5 is n-propyl. In one embodiments, ZA is SO2N(iso-propyl) and BR5 is iso-prpopyl.

In some embodiments, ZA is CH2NHSO2 and BR5 is an optionally substituted C1-6 aliphatic. In some embodiments, ZA is CH2NHSO2 and BR5 is an optionally substituted straight or branched C1-6 alkyl. In one embodiment, ZA is CH2NHSO2 and BR5 is methyl, ethyl, n-propyl, iso-propyl, or n-butyl. In some embodiments, ZA is CH2N(C1-6 aliphatic)SO2 and BR5 is an optionally substituted C1-6 aliphatic. In some embodiments, ZA is CH2N(C1-6 aliphatic)SO2 and BR5 is an optionally substituted straight or branched C1-6 alkyl. In one embodiment, ZA is CH2N(CH3)SO2 and BR5 is methyl, ethyl, n-propyl, iso-propyl, or n-butyl.

In one embodiment, ZA is SO and BR5 is methyl. In one embodiment, ZA is SO2 and BR5 is OH. In some embodiments, ZA is SO2 and BR5 is an optionally substituted straight or branched C1-6 aliphatic or an optionally substituted 3-8 membered heterocyclic, having 1, 2, or 3 ring members independently selected from the group consisting of nitrogen (including NH and NBRX), oxygen, or sulfur (including S, SO, and SO2). In some embodiments, ZA is SO2 and BR5 is straight or branched C1-6 alkyl or 3-8 membered heterocycloaliphatic; each of which is optionally substituted with 1, 2, or 3 of oxo, halo, hydroxy, or an optionally substituted group selected from C1-6 aliphatic, aryl, heteroaryl, carbonyl, amino, and carboxy. In one embodiment, ZA is SO2 and BR5 is methyl, ethyl, or iso-propyl. In some embodiments, ZA is SO2 and examples of BR5 include but are not limited to:

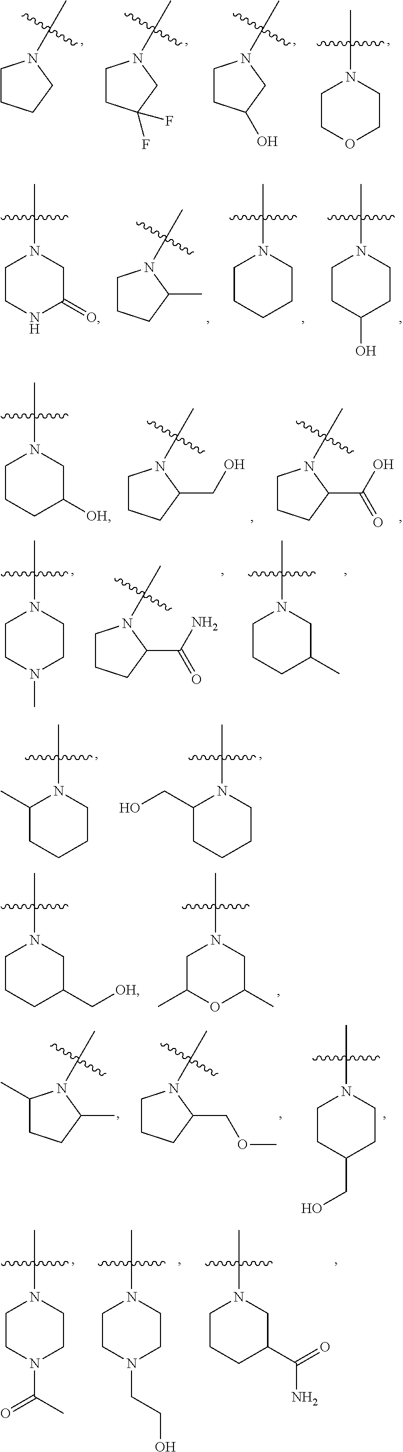

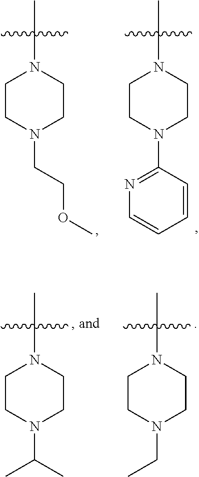

In one embodiment, ZA is CO and BR5 is an optionally substituted amino, an optionally substituted C1-6 straight or branched aliphatic, or an optionally substituted 3-8 membered heterocyclic, having 1, 2, or 3 ring members independently selected from the group consisting of nitrogen (including NH and NBRX), oxygen, or sulfur (including S, SO, and SO2). In one embodiment, ZA is CO and BR5 is di-(2-methoxyethyl)amino or di-(2-hydroxyethyl)amino. In some embodiments, ZA is CO and BR5 is straight or branched C1-6 alkyl or 3-8 membered heterocycloaliphatic each of which is optionally substituted with 1, 2, or 3 of oxo, halo, hydroxy, or an optionally substituted group selected from C1-6 aliphatic, aryl, heteroaryl, carbonyl, amino, and carboxy. In one embodiment, ZA is CO and BR5 is

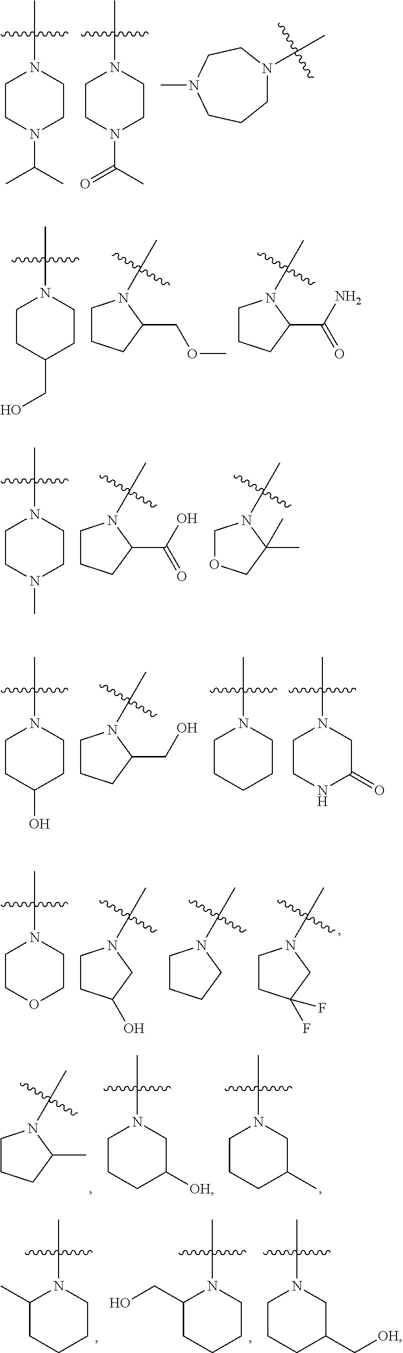

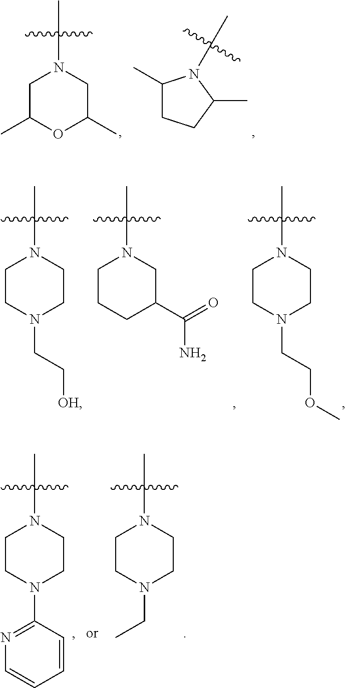

In some embodiments, ZA is NHCO and BR5 is an optionally substituted group selected from C1-6 aliphatic, C1-6 alkoxy, amino, and heterocycloaliphatic. In one embodiment, ZA is NHCO and BR5 is C1-6 alkyl, C1-6 alkoxy, amino, or 3-8 membered heterocycloalkyl having 1, 2, or 3 ring member independently selected from nitrogen (including NH and NBRX), oxygen, or sulfur (including S, SO, and SO2); wherein said alkyl, alkoxy, amino or heterocycloalkyl each is optionally substituted with 1, 2, or 3 groups independently selected from oxo, halo, hydroxy, or an optionally substituted group selected from C1-6 aliphatic, 3-8 membered heterocycloaliphatic, alkoxy, carbonyl, amino, and carboxy. In one embodiment, ZA is NHCO and BR5 is methyl, methoxymethyl, hydroxymethyl, (morpholin-4-yl)-methyl, CH2COOH, ethoxy, dimethylamino, or morpholin-4-yl.

In some embodiments, one BRA not attached at the carbon carbon 3″ or 4″ is selected from the group consisting of H, BRB, halo, —OH, —(CH2)rNBRBBRB, —(CH2)r-OBRB, —SO2-BRB, —NBRB—SO2-BRB, —SO2NBRBBRB, —C(O)BRB, —C(O)OBRB, —OC(O)OBRB, —NBRBC(O)OBRB, and —C(O)NBRBBRB; wherein r is 0, 1, or 2; and each BRB is independently hydrogen, an optionally substituted C1-8 aliphatic group, an optionally substituted cycloaliphatic, an optionally substituted heterocycloaliphatic, an optionally substituted aryl, or an optionally substituted heteroaryl. In other embodiments, one BRA not attached at the carbon carbon 3″ or 4″ is selected from the group consisting of H, C1-6 aliphatic, C3-8 cycloaliphatic, 3-8 membered heterocycloaliphatic, C6-10 aryl, 5-8 membered heteroaryl, halo, —CN, —NH2, —NH(C1-6 aliphatic), —N(C1-6 aliphatic)₂, —CH2-N(C1-6 aliphatic)2, —CH2-(heteroaryl), —CH2-NH(C1-6 aliphatic), —CH2NH2, —OH, —O(C1-6 aliphatic), —CH2OH, —CH2-O(C1-6 aliphatic), —SO2(C1-6 aliphatic), —N(C1-6 aliphatic)-SO2(C1-6 aliphatic), —NH—SO2(C1-6 aliphatic), —SO2NH2, —SO2NH(C1-6 aliphatic), —SO2N(C1-6 aliphatic)2, —C(O)(C1-6 aliphatic), —C(O)O(C1-6 aliphatic), —C(O)OH, —OC(O)O(C1-6 aliphatic), —NHC(O)(C1-6 aliphatic), —NHC(O)O(C1-6 aliphatic), —N(C1-6 aliphatic)C(O)O(C1-6 aliphatic), —C(O)NH2, and —C(O)N(C1-6 aliphatic)2. In several examples, BRA2 is selected from the group consisting of H, C1-6 aliphatic, 5-8 membered heteroaryl, halo, —CN, —NH2, —CH2NH2, —OH, —O(C1-6 aliphatic), —CH2OH, —CH2-(5-8 membered heteroaryl), —SO2(C1-6 aliphatic), —NH—SO2(C1-6 aliphatic), —C(O)O(C1-6 aliphatic), —C(O)OH, —NHC(O)(C1-6 aliphatic), —C(O)NH2, —C(O)NH(C1-6 aliphatic), and —C(O)N(C1-6 aliphatic)2. For examples, one BRA not attached at the carbon carbon 3″ or 4″ is selected from the group consisting of H, methyl, ethyl, n-propyl, iso-propyl, tert-butyl, tetrazol-5-yl, F, Cl, CN, —NH2, —CH2NH2, —CH2CN, —CH2COOH, —CH2CH2COOH, 1,3-dioxo-isoindolin-2-ylmethyl, —OH, —OCH3, —OCF3, ethoxy, iso-propoxy, n-propoxy, —CH2OH, —CH2CH2OH, —SO2CH3, —NH—SO2CH3, —C(O)OCH3, —C(O)OCH2CH3, —C(O)OH, —NHC(O)CH3, —C(O)NH2, and —C(O)N(CH3)2. In one embodiment, one BRA not attached at the carbon carbon 3″ or 4″ is hydrogen. In another embodiment, one BRA not attached at the carbon carbon 3″ or 4″ is methyl, ethyl, F, Cl, or —OCH3.

In some embodiments, one BRA not attached at the carbon carbon 3″ or 4″ is H, hydroxy, halo, C1-6 alkyl, C1-6 alkoxy, C3-4 cycloalkyl, or NH2. In several examples, BRA2 is H, halo, C1-4 alkyl, or C1-4 alkoxy. Examples of one BRA not attached at the carbon carbon 3″ or 4″ include H, F, Cl, methyl, ethyl, and methoxy.

5. Exemplary Compounds

Exemplary Column B compounds of the present invention include, but are not limited to, those illustrated in Table II.B-1 below.

TABLE II.B-1
Examples of Column B compounds of the present invention.

| | |
|---|---|
| 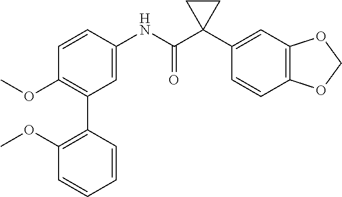 [SMILES: COc1ccccc1-c1cc(NC(=O)C2(c3ccc4c(c3)OCO4)CC2)ccc1OC] | 1 |
| 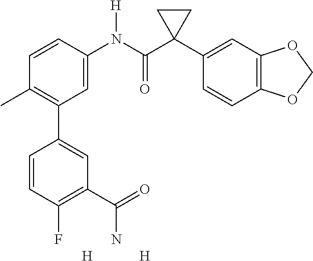 | 2 |
| 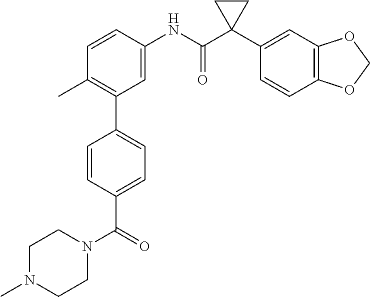 [SMILES: Cc1ccc(NC(=O)C2(c3ccc4c(c3)OCO4)CC2)cc1-c1ccc(C(=O)N2CCN(C)CC2)cc1] | 3 |
| 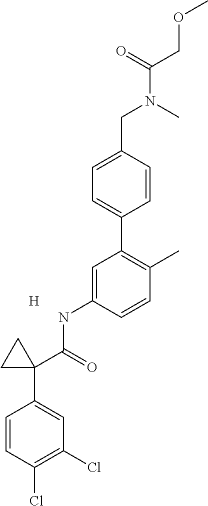 | 4 |
| 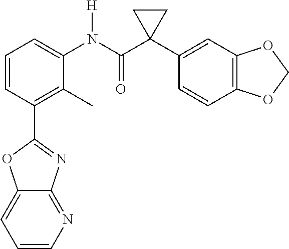 [SMILES: Cc1c(NC(=O)C2(c3ccc4c(c3)OCO4)CC2)cccc1-c1nc2ncccc2o1] | 5 |
| 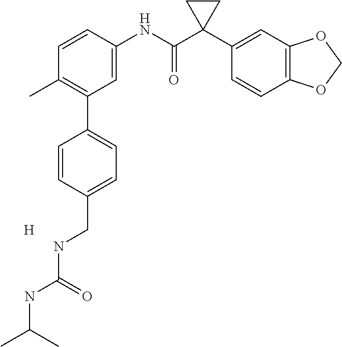 | 6 |
| 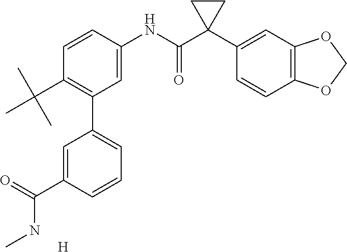 | 7 |
| 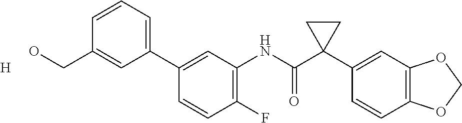 | 8 |
| 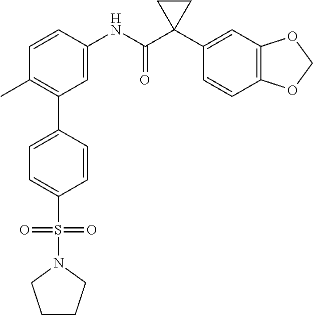 [SMILES: Cc1ccc(NC(=O)C2(c3ccc4c(c3)OCO4)CC2)cc1-c1ccc(S(=O)(=O)N2CCCC2)cc1] | 9 |
| 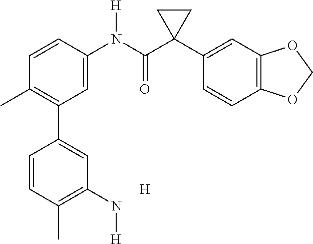 | 10 |
| 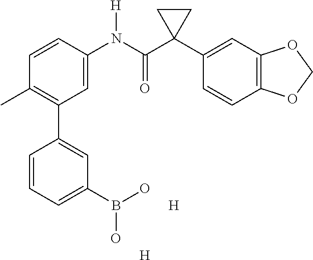 | 11 |
| 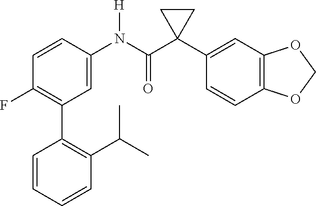 [SMILES: CC(C)c1ccccc1-c1cc(NC(=O)C2(c3ccc4c(c3)OCO4)CC2)ccc1F] | 12 |
| 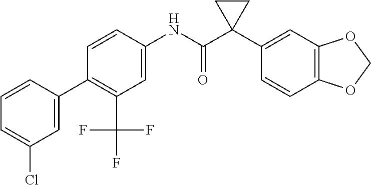 [SMILES: O=C(Nc1ccc(-c2cccc(Cl)c2)c(C(F)(F)F)c1)C1(c2ccc3c(c2)OCO3)CC1] | 13 |
| 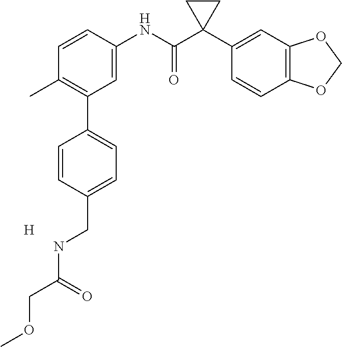 | 14 |
| 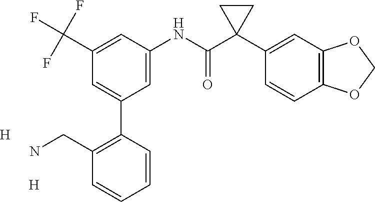 | 15 |
| 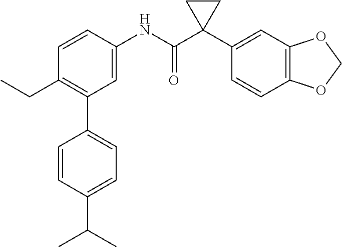 [SMILES: CCc1ccc(NC(=O)C2(c3ccc4c(c3)OCO4)CC2)cc1-c1ccc(C(C)C)cc1] | 16 |
| 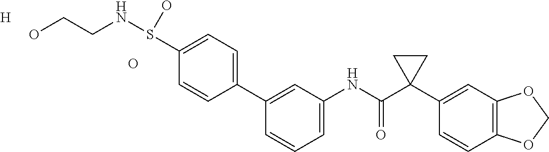 | 17 |
| 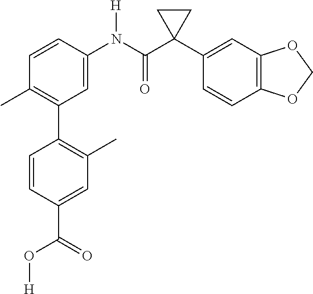 [SMILES: Cc1cc(C(=O)O)ccc1-c1cc(NC(=O)C2(c3ccc4c(c3)OCO4)CC2)ccc1C] | 18 |
| 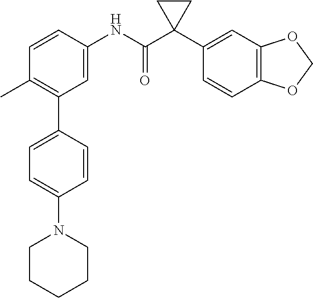 [SMILES: Cc1ccc(NC(=O)C2(c3ccc4c(c3)OCO4)CC2)cc1-c1ccc(N2CCCCC2)cc1] | 19 |
| 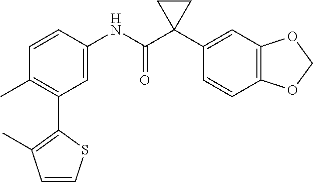 [SMILES: Cc1ccc(NC(=O)C2(c3ccc4c(c3)OCO4)CC2)cc1-c1sccc1C] | 20 |
| 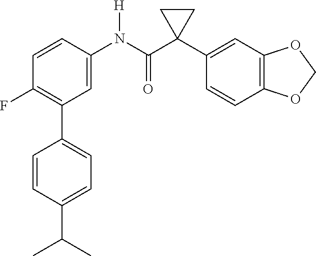 [SMILES: CC(C)c1ccc(-c2cc(NC(=O)C3(c4ccc5c(c4)OCO5)CC3)ccc2F)cc1] | 21 |
| 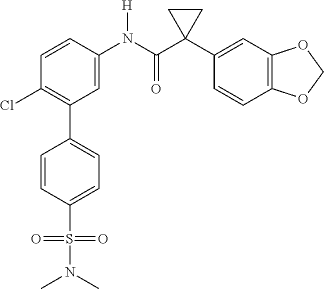 [SMILES: CN(C)S(=O)(=O)c1ccc(-c2cc(NC(=O)C3(c4ccc5c(c4)OCO5)CC3)ccc2Cl)cc1] | 22 |
| 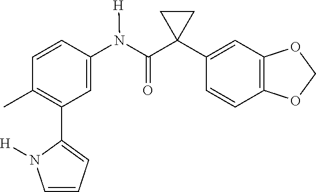 [SMILES: Cc1ccc(NC(=O)C2(c3ccc4c(c3)OCO4)CC2)cc1-c1ccc[nH]1] | 23 |
| 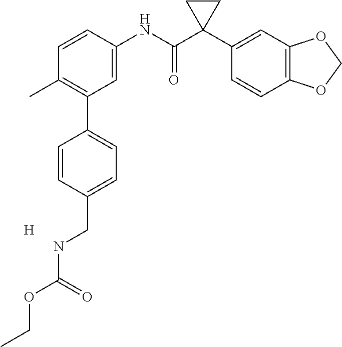 | 24 |
| 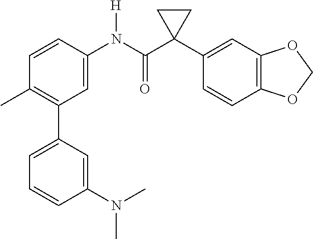 [SMILES: Cc1ccc(NC(=O)C2(c3ccc4c(c3)OCO4)CC2)cc1-c1cccc(N(C)C)c1] | 25 |
| 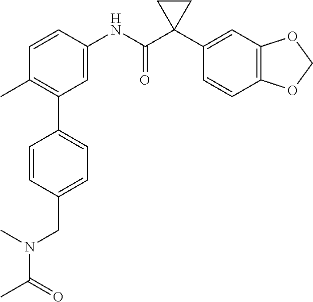 [SMILES: CC(=O)N(C)Cc1ccc(-c2cc(NC(=O)C3(c4ccc5c(c4)OCO5)CC3)ccc2C)cc1] | 26 |
| 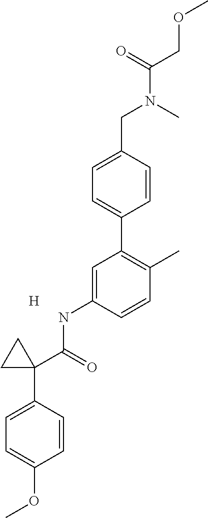 | 27 |
| 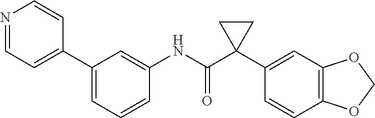 [SMILES: O=C(Nc1cccc(-c2ccncc2)c1)C1(c2ccc3c(c2)OCO3)CC1] | 28 |
| 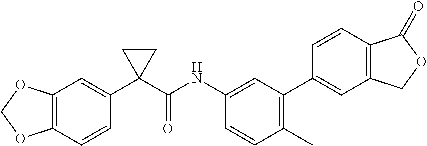 [SMILES: Cc1ccc(NC(=O)C2(c3ccc4c(c3)OCO4)CC2)cc1-c1ccc2c(c1)COC2=O] | 29 |
| 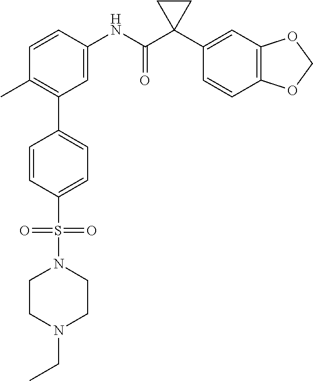 [SMILES: CCN1CCN(S(=O)(=O)c2ccc(-c3cc(NC(=O)C4(c5ccc6c(c5)OCO6)CC4)ccc3C)cc2)CC1] | 30 |
| 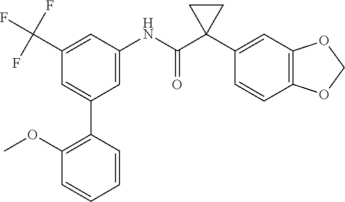 [SMILES: COc1ccccc1-c1cc(NC(=O)C2(c3ccc4c(c3)OCO4)CC2)cc(C(F)(F)F)c1] | 31 |
| 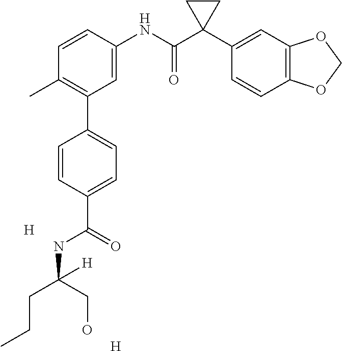 | 32 |
| 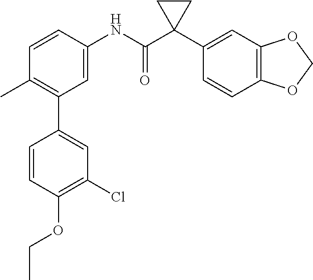 [SMILES: CCOc1ccc(-c2cc(NC(=O)C3(c4ccc5c(c4)OCO5)CC3)ccc2C)cc1Cl] | 33 |
| 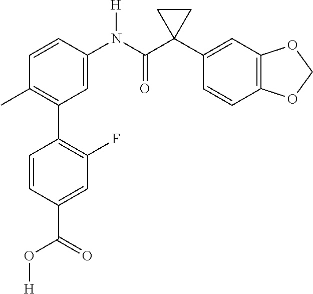 [SMILES: Cc1ccc(NC(=O)C2(c3ccc4c(c3)OCO4)CC2)cc1-c1ccc(C(=O)O)cc1F] | 34 |
| 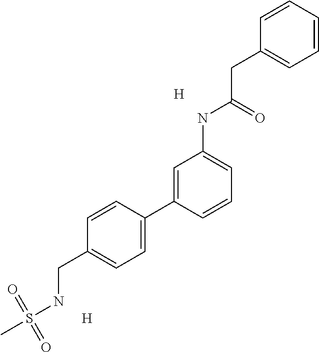 | 35 |
| 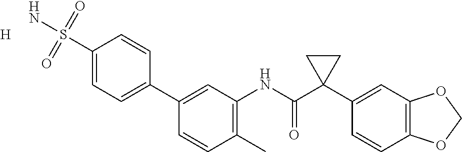 | 36 |
| 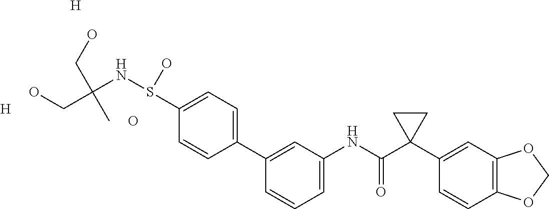 | 37 |
| 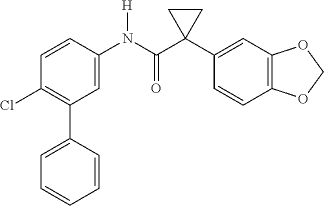 [SMILES: O=C(Nc1ccc(Cl)c(-c2ccccc2)c1)C1(c2ccc3c(c2)OCO3)CC1] | 38 |
| 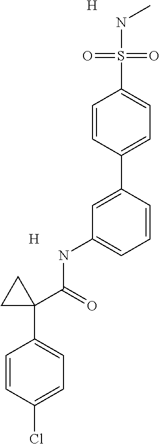 | 39 |
| 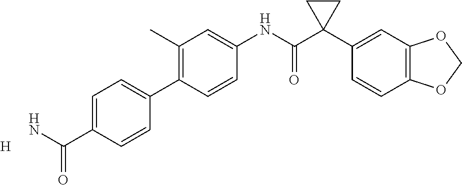 | 40 |
| 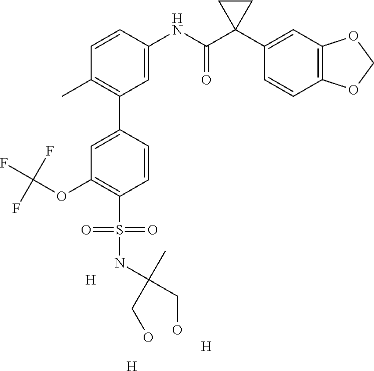 | 41 |
| 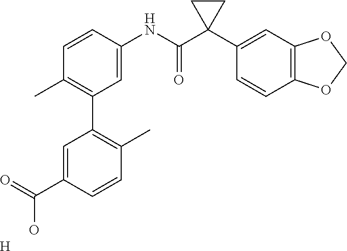 | 42 |
| 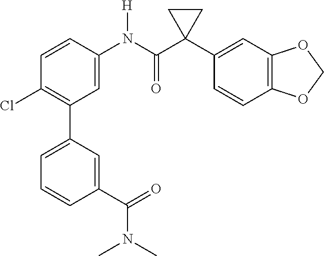 [SMILES: CN(C)C(=O)c1cccc(-c2cc(NC(=O)C3(c4ccc5c(c4)OCO5)CC3)ccc2Cl)c1] | 43 |
| 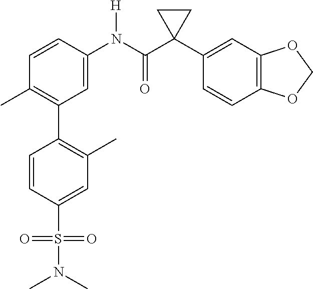 [SMILES: Cc1cc(S(=O)(=O)N(C)C)ccc1-c1cc(NC(=O)C2(c3ccc4c(c3)OCO4)CC2)ccc1C] | 44 |
| 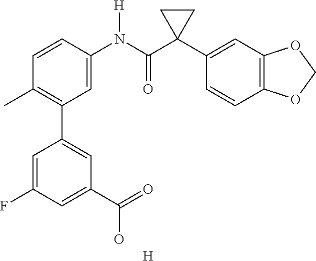 | 45 |
| 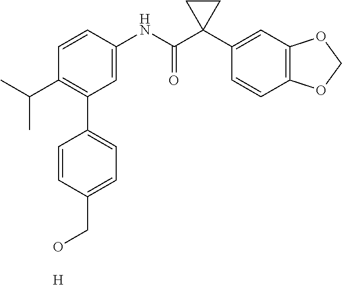 | 46 |
| 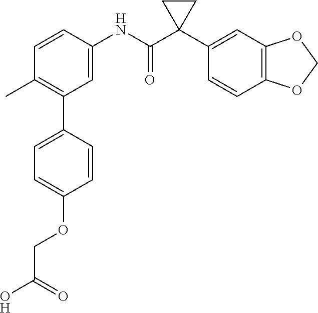 [SMILES: Cc1ccc(NC(=O)C2(c3ccc4c(c3)OCO4)CC2)cc1-c1ccc(OCC(=O)O)cc1] | 47 |
| 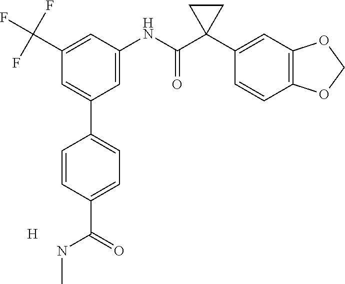 | 48 |
| 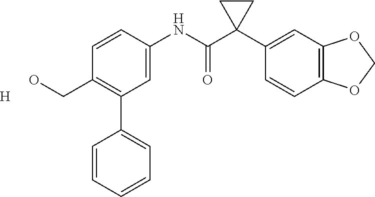 | 49 |
| 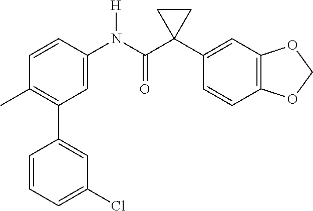 [SMILES: Cc1ccc(NC(=O)C2(c3ccc4c(c3)OCO4)CC2)cc1-c1cccc(Cl)c1] | 50 |
| 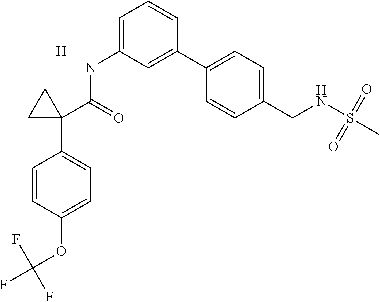 | 51 |
| 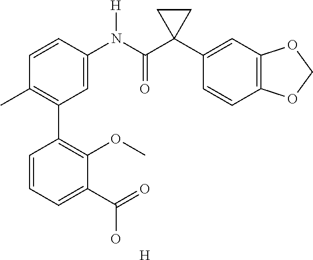 | 52 |
| 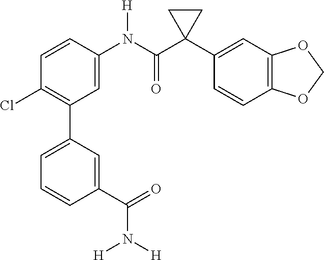 [SMILES: NC(=O)c1cccc(-c2cc(NC(=O)C3(c4ccc5c(c4)OCO5)CC3)ccc2Cl)c1] | 53 |
| 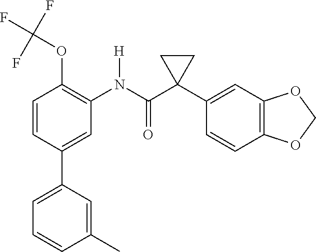 [SMILES: Cc1cccc(-c2ccc(OC(F)(F)F)c(NC(=O)C3(c4ccc5c(c4)OCO5)CC3)c2)c1] | 54 |
| 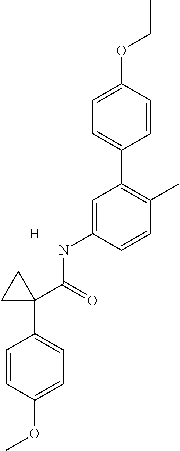 | 55 |
| 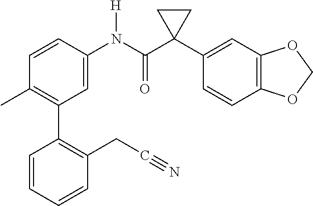 [SMILES: Cc1ccc(NC(=O)C2(c3ccc4c(c3)OCO4)CC2)cc1-c1ccccc1CC#N] | 56 |
| 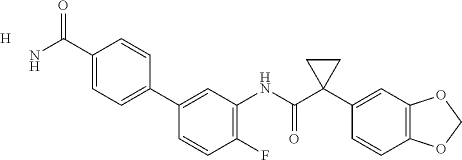 | 57 |
| 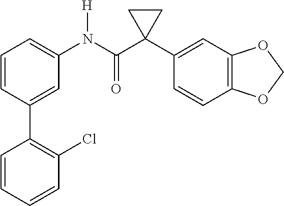 [SMILES: O=C(Nc1cccc(-c2ccccc2Cl)c1)C1(c2ccc3c(c2)OCO3)CC1] | 58 |
| 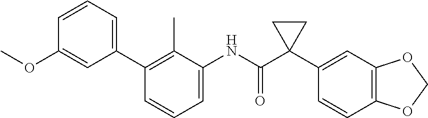 [SMILES: COc1cccc(-c2cccc(NC(=O)C3(c4ccc5c(c4)OCO5)CC3)c2C)c1] | 59 |
| 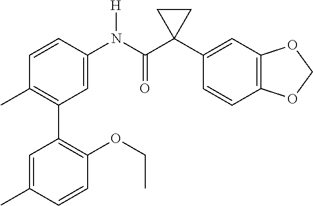 [SMILES: CCOc1ccc(C)cc1-c1cc(NC(=O)C2(c3ccc4c(c3)OCO4)CC2)ccc1C] | 60 |
| 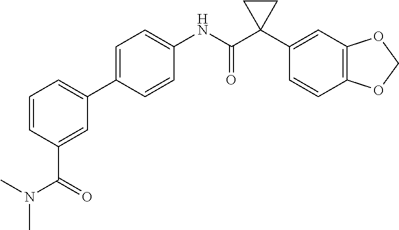 [SMILES: CN(C)C(=O)c1cccc(-c2ccc(NC(=O)C3(c4ccc5c(c4)OCO5)CC3)cc2)c1] | 61 |
| 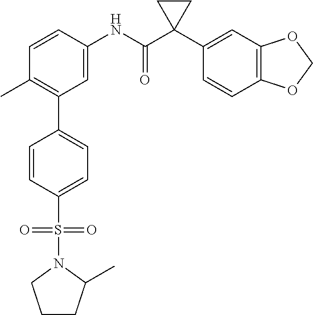 [SMILES: Cc1ccc(NC(=O)C2(c3ccc4c(c3)OCO4)CC2)cc1-c1ccc(S(=O)(=O)N2CCCC2C)cc1] | 62 |
| 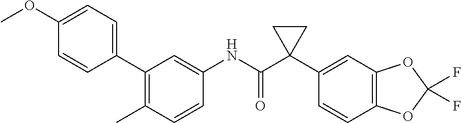 [SMILES: COc1ccc(-c2cc(NC(=O)C3(c4ccc5c(c4)OC(F)(F)O5)CC3)ccc2C)cc1] | 63 |
| 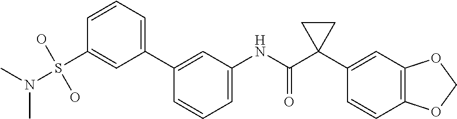 [SMILES: CN(C)S(O)(O)c1cccc(-c2cccc(NC(=O)C3(c4ccc5c(c4)OCO5)CC3)c2)c1] | 64 |
| 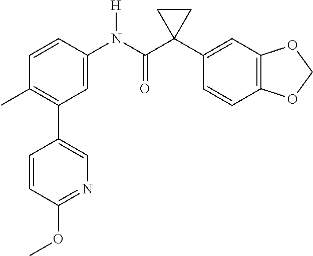 [SMILES: COc1ccc(-c2cc(NC(=O)C3(c4ccc5c(c4)OCO5)CC3)ccc2C)cn1] | 65 |
| 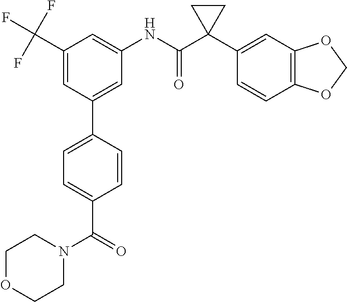 [SMILES: O=C(c1ccc(-c2cc(NC(=O)C3(c4ccc5c(c4)OCO5)CC3)cc(C(F)(F)F)c2)cc1)N1CCOCC1] | 66 |
| 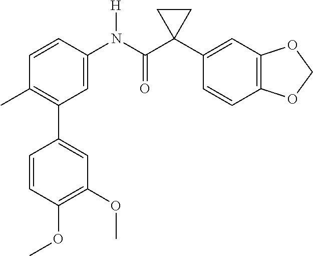 [SMILES: COc1ccc(-c2cc(NC(=O)C3(c4ccc5c(c4)OCO5)CC3)ccc2C)cc1OC] | 67 |
| 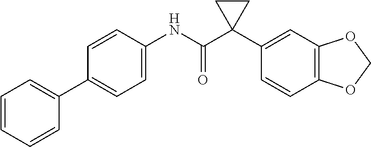 [SMILES: O=C(Nc1ccc(-c2ccccc2)cc1)C1(c2ccc3c(c2)OCO3)CC1] | 68 |
| 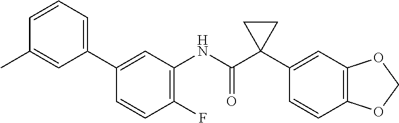 [SMILES: Cc1cccc(-c2ccc(F)c(NC(=O)C3(c4ccc5c(c4)OCO5)CC3)c2)c1] | 69 |
| 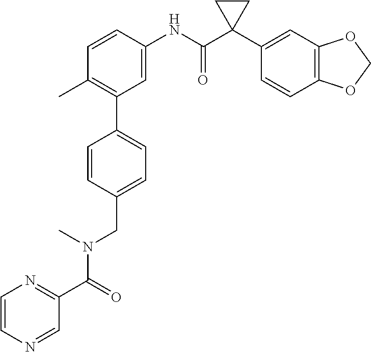 [SMILES: Cc1ccc(NC(=O)C2(c3ccc4c(c3)OCO4)CC2)cc1-c1ccc(CN(C)C(=O)c2cnccn2)cc1] | 70 |
| 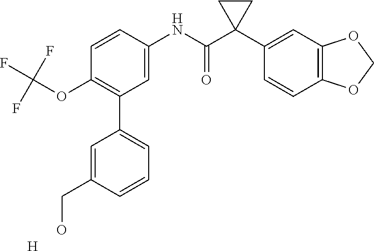 | 71 |
| 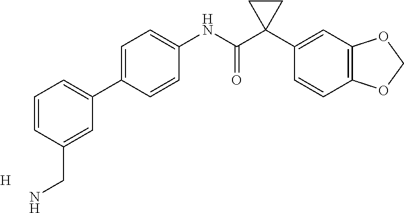 | 72 |
| 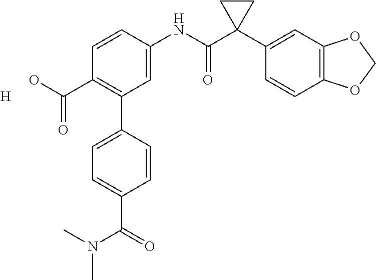 | 73 |
| 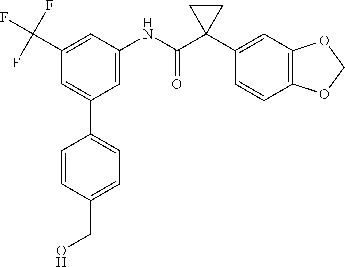 [SMILES: O=C(Nc1cc(-c2ccc(CO)cc2)cc(C(F)(F)F)c1)C1(c2ccc3c(c2)OCO3)CC1] | 74 |
| 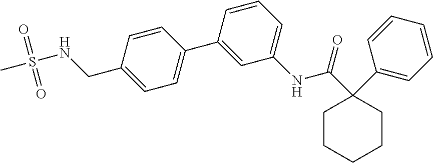 [SMILES: CS(=O)(=O)NCc1ccc(-c2cccc(NC(=O)C3(c4ccccc4)CCCCC3)c2)cc1] | 75 |
| 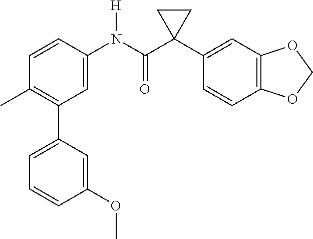 [SMILES: COc1cccc(-c2cc(NC(=O)C3(c4ccc5c(c4)OCO5)CC3)ccc2C)c1] | 76 |
| 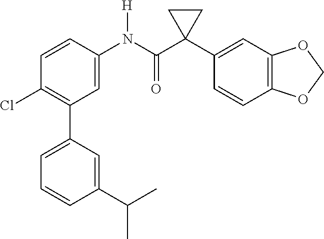 [SMILES: CC(C)c1cccc(-c2cc(NC(=O)C3(c4ccc5c(c4)OCO5)CC3)ccc2Cl)c1] | 77 |
| 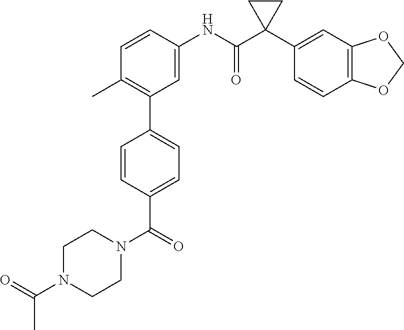 [SMILES: CC(=O)N1CCN(C(=O)c2ccc(-c3cc(NC(=O)C4(c5ccc6c(c5)OCO6)CC4)ccc3C)cc2)CC1] | 78 |
| 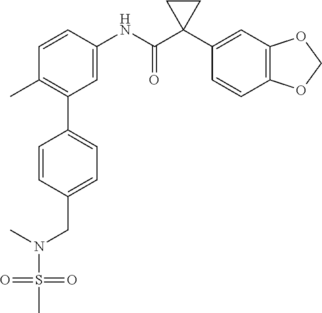 [SMILES: Cc1ccc(NC(=O)C2(c3ccc4c(c3)OCO4)CC2)cc1-c1ccc(CN(C)S(C)(=O)=O)cc1] | 79 |
| 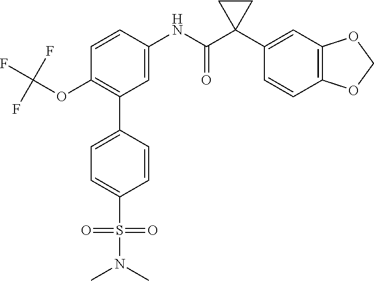 [SMILES: CN(C)S(=O)(=O)c1ccc(-c2cc(NC(=O)C3(c4ccc5c(c4)OCO5)CC3)ccc2OC(F)(F)F)cc1] | 80 |
| 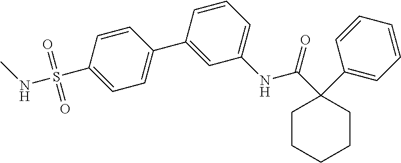 [SMILES: CNS(=O)(=O)c1ccc(-c2cccc(NC(=O)C3(c4ccccc4)CCCCC3)c2)cc1] | 81 |
| 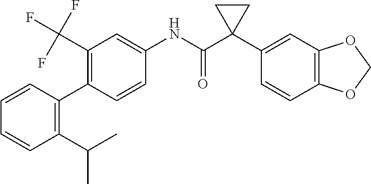 [SMILES: CC(C)c1ccccc1-c1ccc(NC(=O)C2(c3ccc4c(c3)OCO4)CC2)cc1C(F)(F)F] | 82 |
| 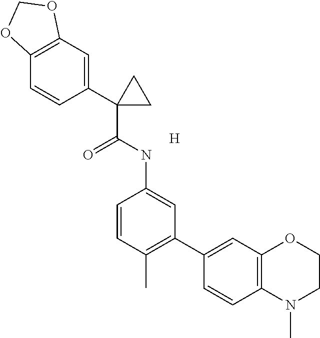 | 83 |
| 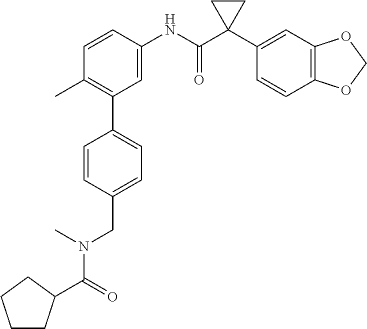 [SMILES: Cc1ccc(NC(=O)C2(c3ccc4c(c3)OCO4)CC2)cc1-c1ccc(CN(C)C(=O)C2CCCC2)cc1] | 84 |
| 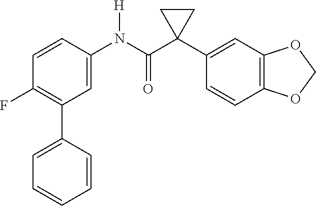 [SMILES: O=C(Nc1ccc(F)c(-c2ccccc2)c1)C1(c2ccc3c(c2)OCO3)CC1] | 85 |
| 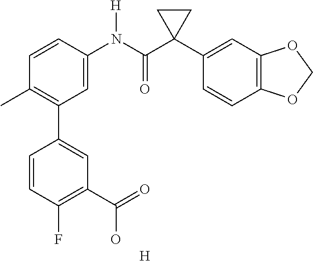 | 86 |
| 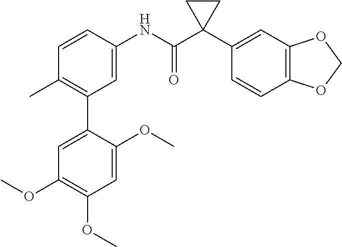 [SMILES: COc1cc(OC)c(-c2cc(NC(=O)C3(c4ccc5c(c4)OCO5)CC3)ccc2C)cc1OC] | 87 |
| 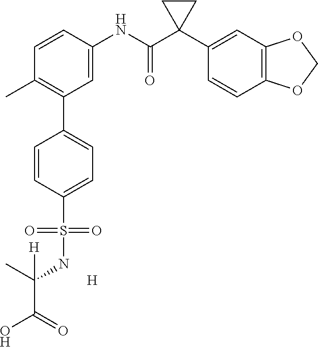 | 88 |
| 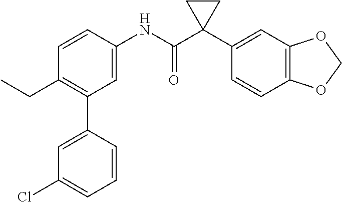 [SMILES: CCc1ccc(NC(=O)C2(c3ccc4c(c3)OCO4)CC2)cc1-c1cccc(Cl)c1] | 89 |
| 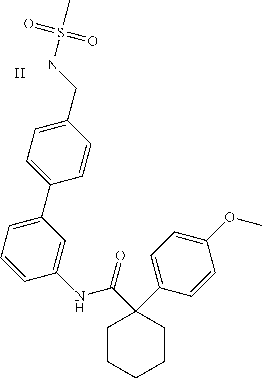 | 90 |
| 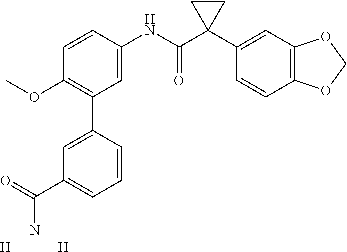 | 91 |
| 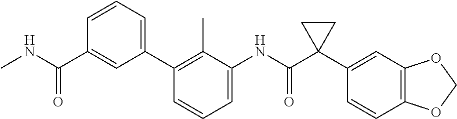 [SMILES: CNC(=O)c1cccc(-c2cccc(NC(=O)C3(c4ccc5c(c4)OCO5)CC3)c2C)c1] | 92 |
| 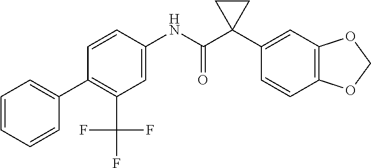 [SMILES: O=C(Nc1ccc(-c2ccccc2)c(C(F)(F)F)c1)C1(c2ccc3c(c2)OCO3)CC1] | 93 |
| 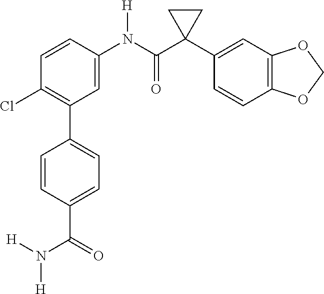 [SMILES: NC(=O)c1ccc(-c2cc(NC(=O)C3(c4ccc5c(c4)OCO5)CC3)ccc2Cl)cc1] | 94 |
| 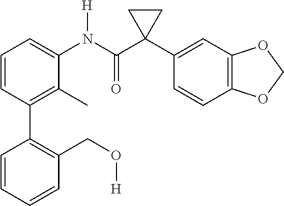 [SMILES: Cc1c(NC(=O)C2(c3ccc4c(c3)OCO4)CC2)cccc1-c1ccccc1CO] | 95 |
| 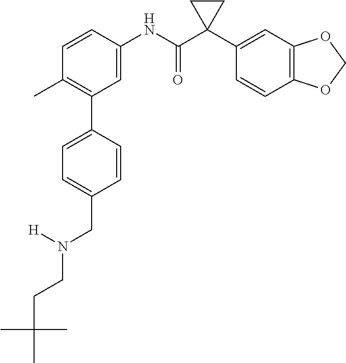 [SMILES: Cc1ccc(NC(=O)C2(c3ccc4c(c3)OCO4)CC2)cc1-c1ccc(CNCCC(C)(C)C)cc1] | 96 |
| 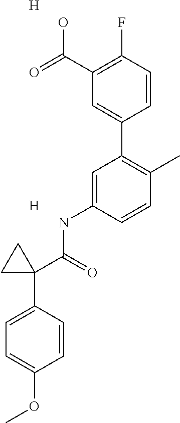 | 97 |
| 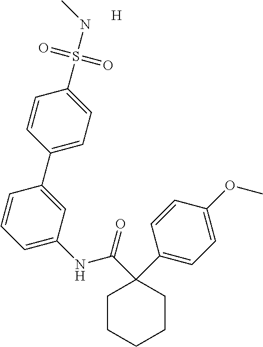 | 98 |
| 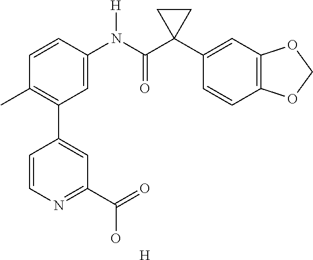 | 99 |
| 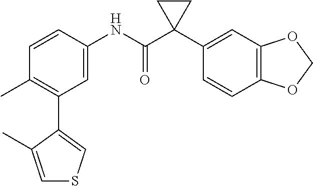 [SMILES: Cc1cscc1-c1cc(NC(=O)C2(c3ccc4c(c3)OCO4)CC2)ccc1C] | 100 |
| 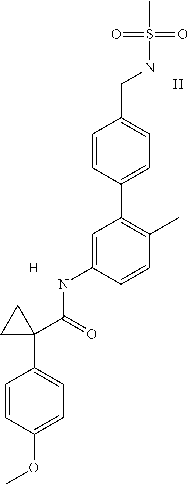 | 101 |
| 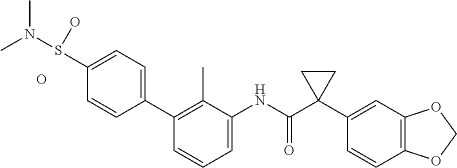 | 102 |
| 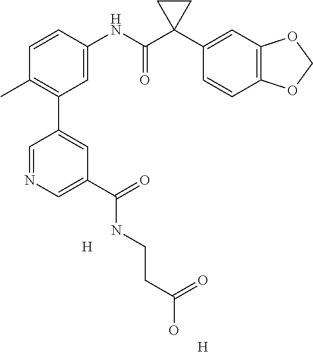 | 103 |
| 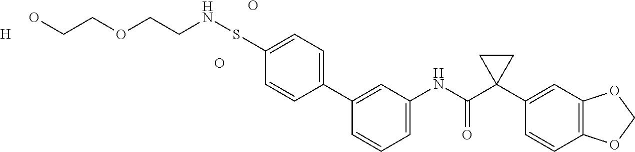 | 104 |
| 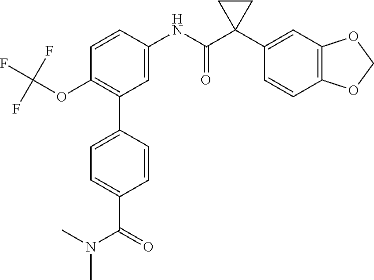 [SMILES: CN(C)C(=O)c1ccc(-c2cc(NC(=O)C3(c4ccc5c(c4)OCO5)CC3)ccc2OC(F)(F)F)cc1] | 105 |
| 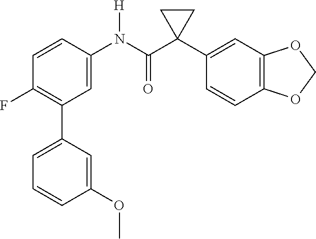 [SMILES: COc1cccc(-c2cc(NC(=O)C3(c4ccc5c(c4)OCO5)CC3)ccc2F)c1] | 106 |
| 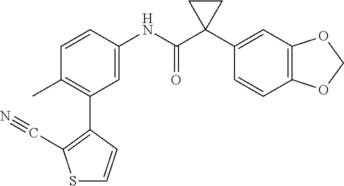 [SMILES: Cc1ccc(NC(=O)C2(c3ccc4c(c3)OCO4)CC2)cc1-c1ccsc1C#N] | 107 |
| 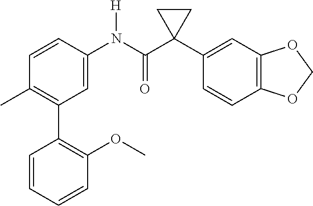 [SMILES: COc1ccccc1-c1cc(NC(=O)C2(c3ccc4c(c3)OCO4)CC2)ccc1C] | 108 |
| 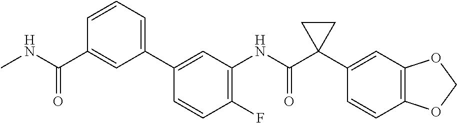 [SMILES: CNC(=O)c1cccc(-c2ccc(F)c(NC(=O)C3(c4ccc5c(c4)OCO5)CC3)c2)c1] | 109 |
| 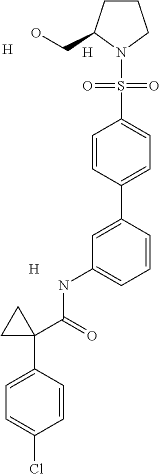 | 110 |
| 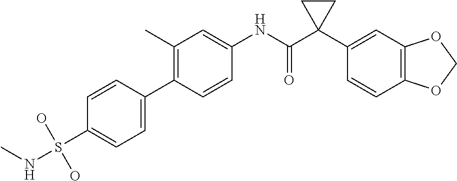 [SMILES: CNS(O)(O)c1ccc(-c2ccc(NC(=O)C3(c4ccc5c(c4)OCO5)CC3)cc2C)cc1] | 111 |
| 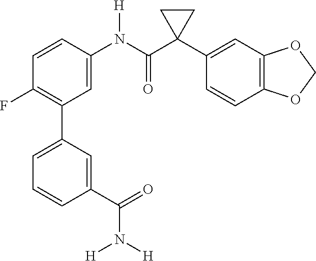 [SMILES: NC(=O)c1cccc(-c2cc(NC(=O)C3(c4ccc5c(c4)OCO5)CC3)ccc2F)c1] | 112 |
| 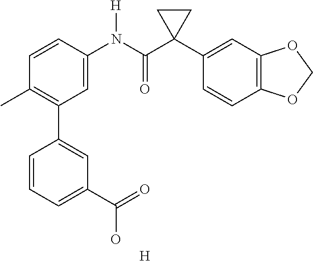 | 113 |
| 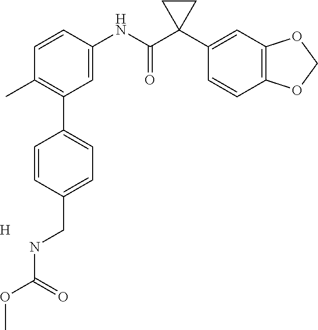 | 114 |
| 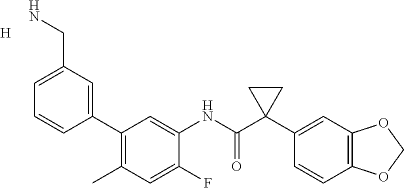 | 115 |
| 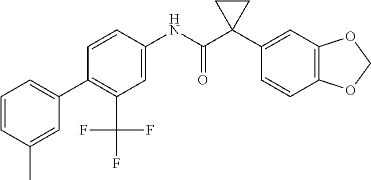 [SMILES: Cc1cccc(-c2ccc(NC(=O)C3(c4ccc5c(c4)OCO5)CC3)cc2C(F)(F)F)c1] | 116 |
| 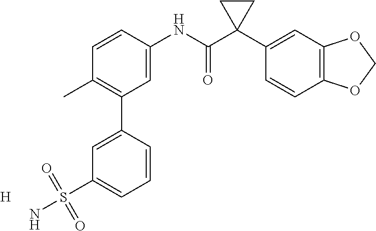 | 117 |
| 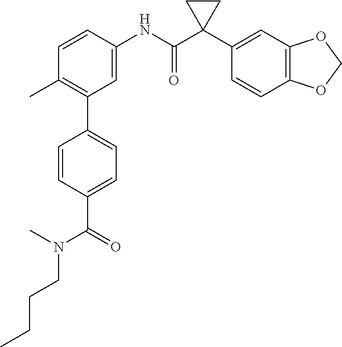 [SMILES: CCCCN(C)C(=O)c1ccc(-c2cc(NC(=O)C3(c4ccc5c(c4)OCO5)CC3)ccc2C)cc1] | 118 |
| 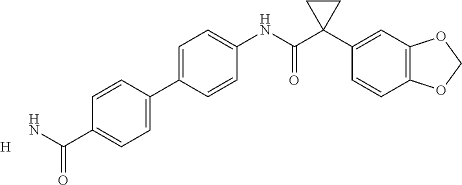 | 119 |
| 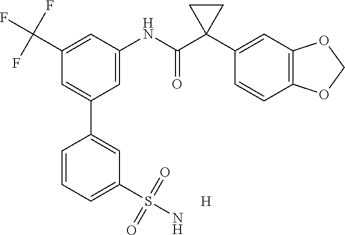 | 120 |
| 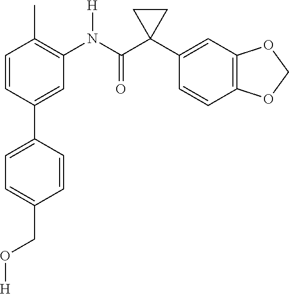 [SMILES: Cc1ccc(-c2ccc(CO)cc2)cc1NC(=O)C1(c2ccc3c(c2)OCO3)CC1] | 121 |
| 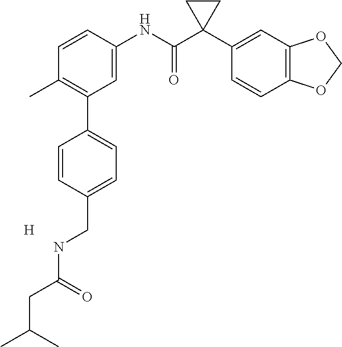 | 122 |
| 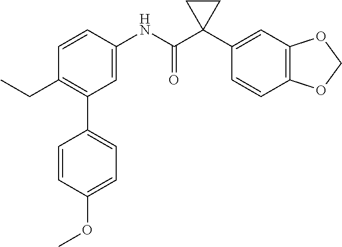 [SMILES: CCc1ccc(NC(=O)C2(c3ccc4c(c3)OCO4)CC2)cc1-c1ccc(OC)cc1] | 123 |
| 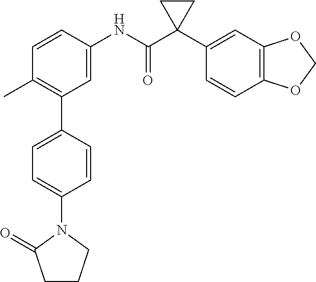 [SMILES: Cc1ccc(NC(=O)C2(c3ccc4c(c3)OCO4)CC2)cc1-c1ccc(N2CCCC2=O)cc1] | 124 |
| 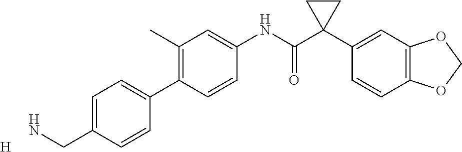 | 125 |
| 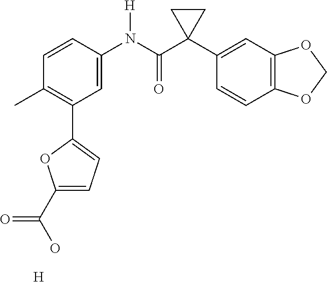 | 126 |
| 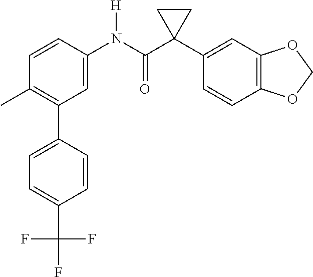 [SMILES: Cc1ccc(NC(=O)C2(c3ccc4c(c3)OCO4)CC2)cc1-c1ccc(C(F)(F)F)cc1] | 127 |
| 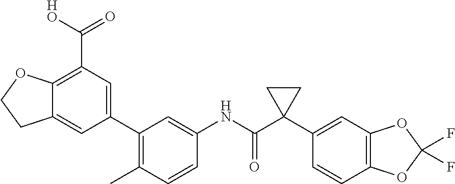 [SMILES: Cc1ccc(NC(=O)C2(c3ccc4c(c3)OC(F)(F)O4)CC2)cc1-c1cc2c(c(C(=O)O)c1)OCC2] | 128 |
| 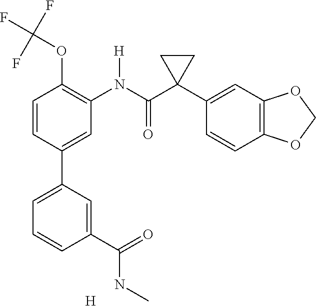 | 129 |
| 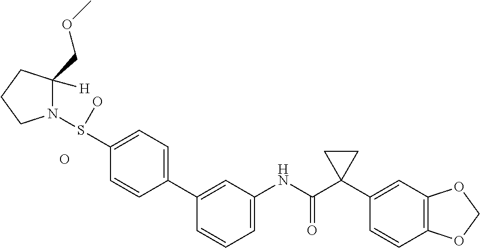 | 130 |
| 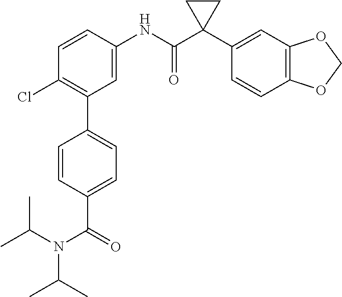 [SMILES: CC(C)N(C(=O)c1ccc(-c2cc(NC(=O)C3(c4ccc5c(c4)OCO5)CC3)ccc2Cl)cc1)C(C)C] | 131 |
| 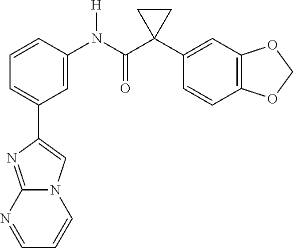 [SMILES: O=C(Nc1cccc(-c2cn3cccnc3n2)c1)C1(c2ccc3c(c2)OCO3)CC1] | 132 |
| 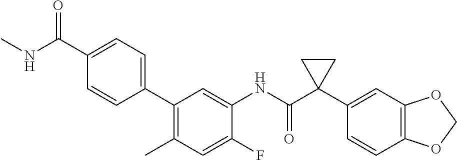 [SMILES: CNC(=O)c1ccc(-c2cc(NC(=O)C3(c4ccc5c(c4)OCO5)CC3)c(F)cc2C)cc1] | 133 |
| 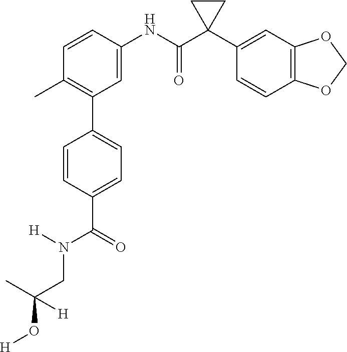 [SMILES: Cc1ccc(NC(=O)C2(c3ccc4c(c3)OCO4)CC2)cc1-c1ccc(C(=O)NC[C@H](C)O)cc1] | 134 |
| 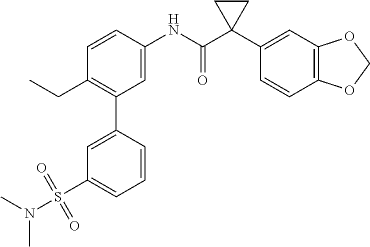 [SMILES: CCc1ccc(NC(=O)C2(c3ccc4c(c3)OCO4)CC2)cc1-c1cccc(S(=O)(=O)N(C)C)c1] | 135 |
| 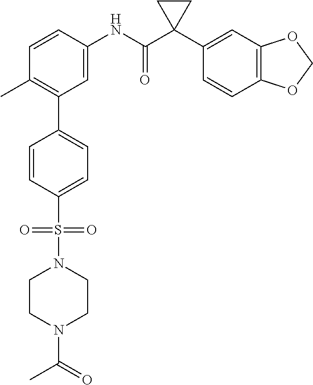 [SMILES: CC(=O)N1CCN(S(=O)(=O)c2ccc(-c3cc(NC(=O)C4(c5ccc6c(c5)OCO6)CC4)ccc3C)cc2)CC1] | 136 |
| 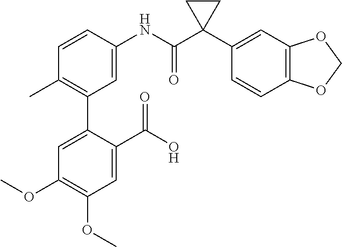 [SMILES: COc1cc(C(=O)O)c(-c2cc(NC(=O)C3(c4ccc5c(c4)OCO5)CC3)ccc2C)cc1OC] | 137 |
| 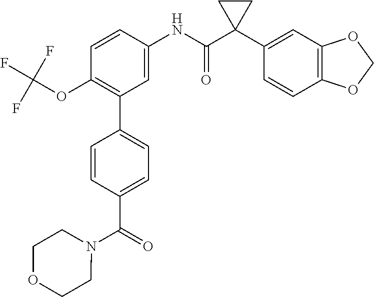 [SMILES: O=C(c1ccc(-c2cc(NC(=O)C3(c4ccc5c(c4)OCO5)CC3)ccc2OC(F)(F)F)cc1)N1CCOCC1] | 138 |
| 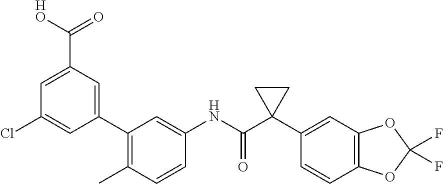 [SMILES: Cc1ccc(NC(=O)C2(c3ccc4c(c3)OC(F)(F)O4)CC2)cc1-c1cc(Cl)cc(C(=O)O)c1] | 139 |
| 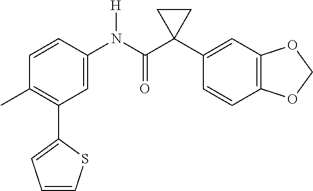 [SMILES: Cc1ccc(NC(=O)C2(c3ccc4c(c3)OCO4)CC2)cc1-c1cccs1] | 140 |
| 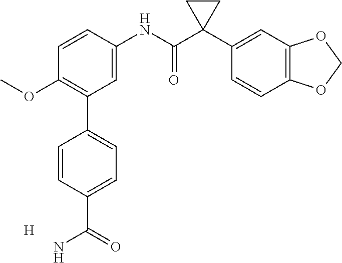 | 141 |
| 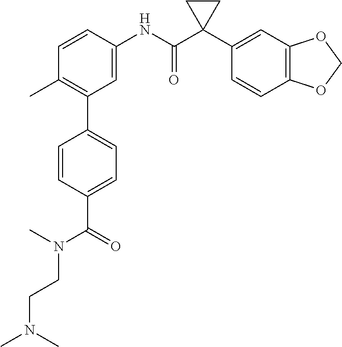 [SMILES: Cc1ccc(NC(=O)C2(c3ccc4c(c3)OCO4)CC2)cc1-c1ccc(C(=O)N(C)CCN(C)C)cc1] | 142 |
| 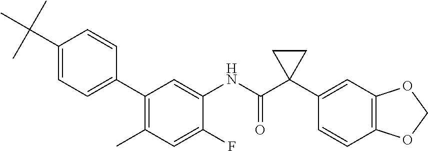 [SMILES: Cc1cc(F)c(NC(=O)C2(c3ccc4c(c3)OCO4)CC2)cc1-c1ccc(C(C)(C)C)cc1] | 143 |
| 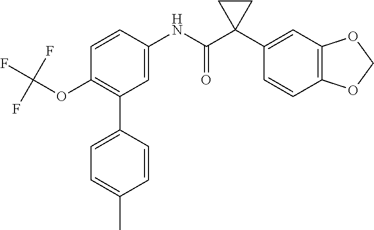 [SMILES: Cc1ccc(-c2cc(NC(=O)C3(c4ccc5c(c4)OCO5)CC3)ccc2OC(F)(F)F)cc1] | 144 |
| 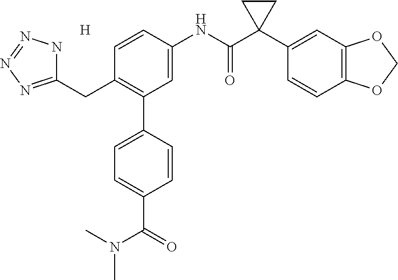 | 145 |
| 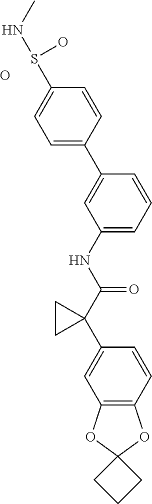 | 146 |
| 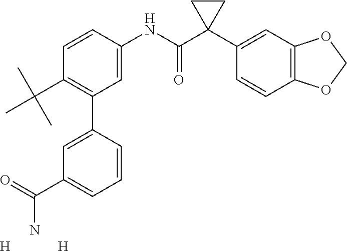 | 147 |
| 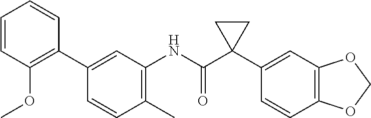 [SMILES: COc1ccccc1-c1ccc(C)c(NC(=O)C2(c3ccc4c(c3)OCO4)CC2)c1] | 148 |
| 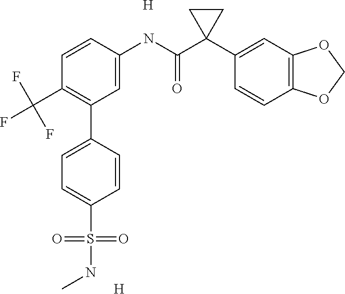 | 149 |
| 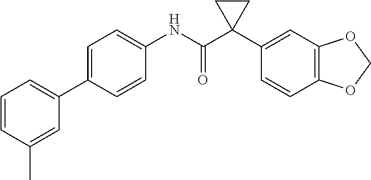 [SMILES: Cc1cccc(-c2ccc(NC(=O)C3(c4ccc5c(c4)OCO5)CC3)cc2)c1] | 150 |
| 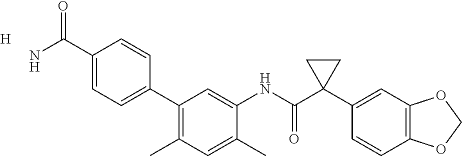 | 151 |
| 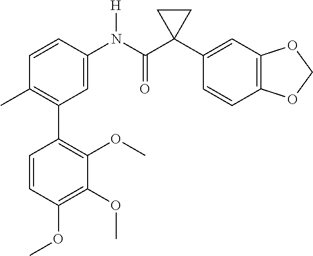 [SMILES: COc1ccc(-c2cc(NC(=O)C3(c4ccc5c(c4)OCO5)CC3)ccc2C)c(OC)c1OC] | 152 |
| 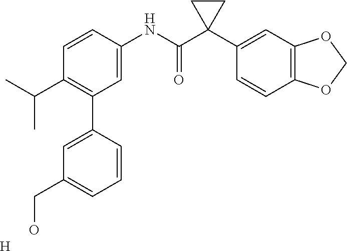 | 153 |
| 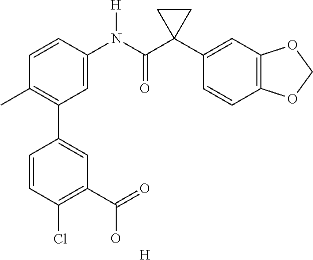 | 154 |
| 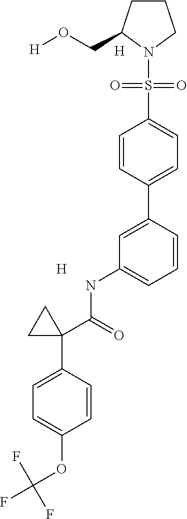 | 155 |
| 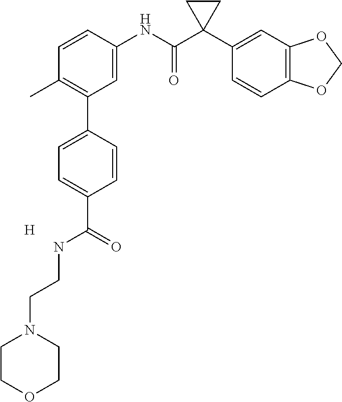 | 156 |
| 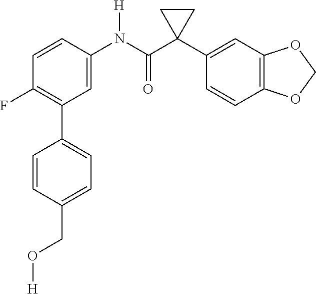 [SMILES: O=C(Nc1ccc(F)c(-c2ccc(CO)cc2)c1)C1(c2ccc3c(c2)OCO3)CC1] | 157 |
| 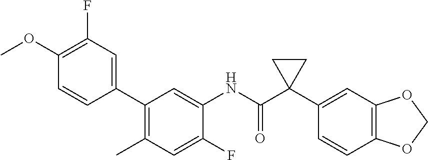 [SMILES: COc1ccc(-c2cc(NC(=O)C3(c4ccc5c(c4)OCO5)CC3)c(F)cc2C)cc1F] | 158 |
| 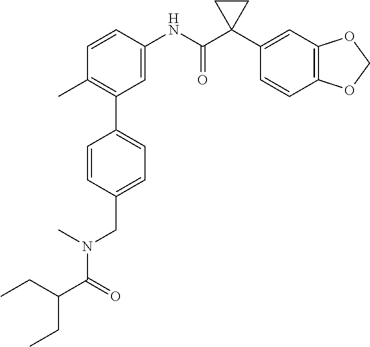 [SMILES: CCC(CC)C(=O)N(C)Cc1ccc(-c2cc(NC(=O)C3(c4ccc5c(c4)OCO5)CC3)ccc2C)cc1] | 159 |
| 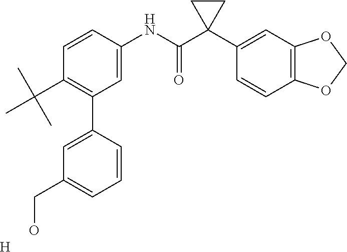 | 160 |
| 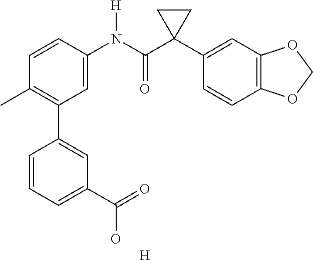 | 161 |
| 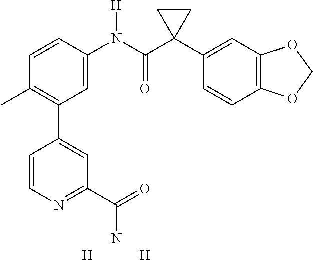 | 162 |
| 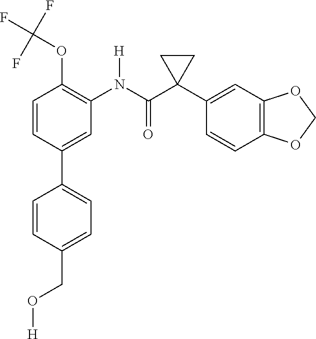 [SMILES: O=C(Nc1cc(-c2ccc(CO)cc2)ccc1OC(F)(F)F)C1(c2ccc3c(c2)OCO3)CC1] | 163 |
| 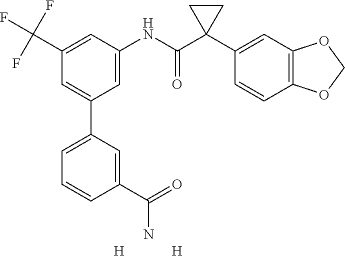 | 164 |
| 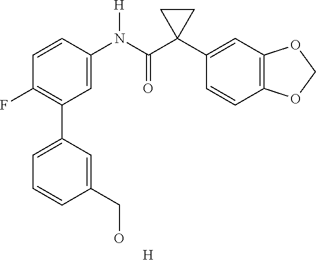 | 165 |
| 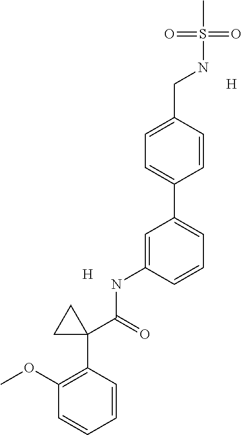 | 166 |
| 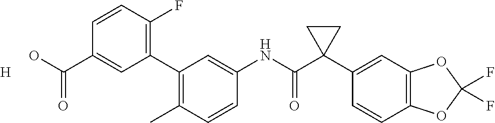 | 167 |
| 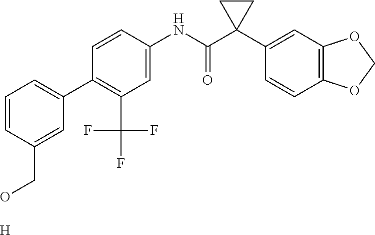 | 168 |
| 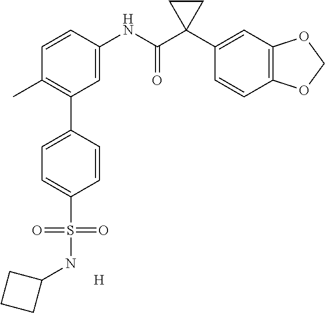 | 169 |
| 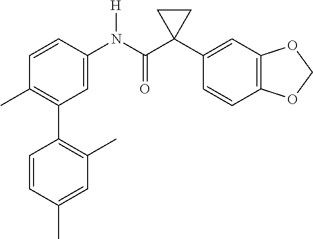 [SMILES: Cc1ccc(-c2cc(NC(=O)C3(c4ccc5c(c4)OCO5)CC3)ccc2C)c(C)c1] | 170 |
| 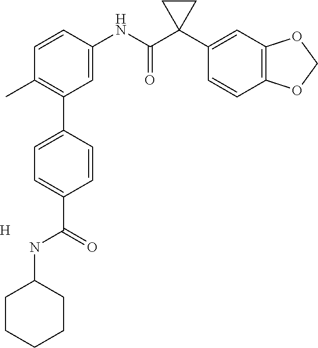 | 171 |
| 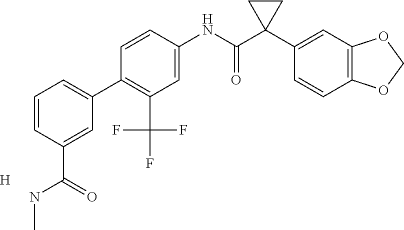 | 172 |
| 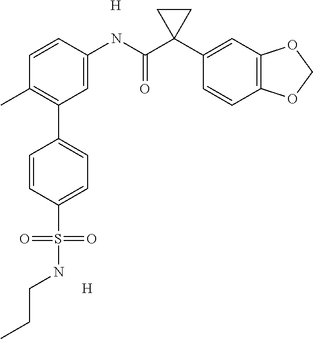 | 173 |
| 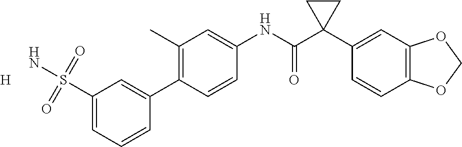 | 174 |
| 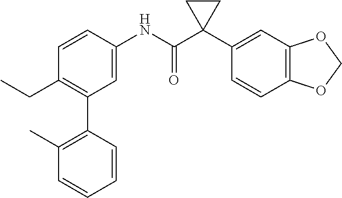 [SMILES: CCc1ccc(NC(=O)C2(c3ccc4c(c3)OCO4)CC2)cc1-c1ccccc1C] | 175 |
| 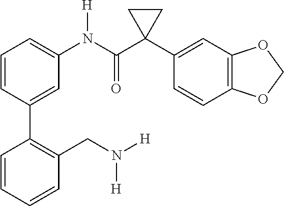 [SMILES: NCc1ccccc1-c1cccc(NC(=O)C2(c3ccc4c(c3)OCO4)CC2)c1] | 176 |
| 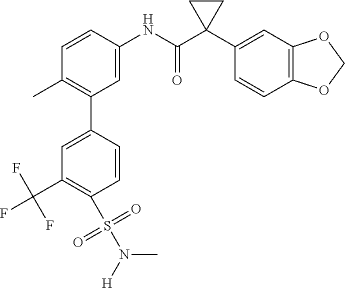 [SMILES: CNS(=O)(=O)c1ccc(-c2cc(NC(=O)C3(c4ccc5c(c4)OCO5)CC3)ccc2C)cc1C(F)(F)F] | 177 |
| 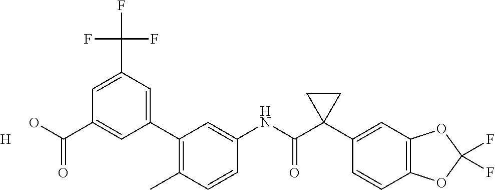 | 178 |
| 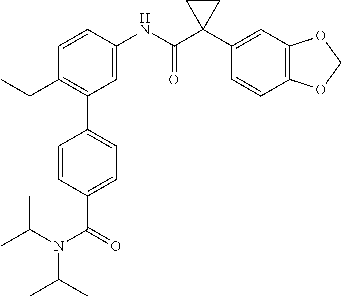 [SMILES: CCc1ccc(NC(=O)C2(c3ccc4c(c3)OCO4)CC2)cc1-c1ccc(C(=O)N(C(C)C)C(C)C)cc1] | 179 |
| 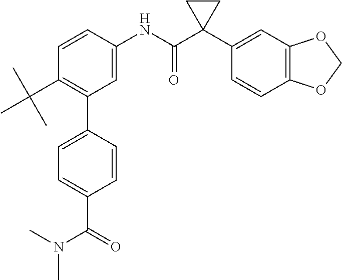 [SMILES: CN(C)C(=O)c1ccc(-c2cc(NC(=O)C3(c4ccc5c(c4)OCO5)CC3)ccc2C(C)(C)C)cc1] | 180 |
| 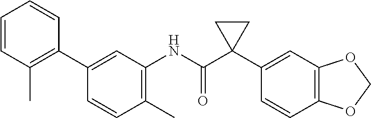 [SMILES: Cc1ccc(-c2ccccc2C)cc1NC(=O)C1(c2ccc3c(c2)OCO3)CC1] | 181 |
| 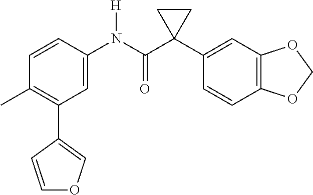 [SMILES: Cc1ccc(NC(=O)C2(c3ccc4c(c3)OCO4)CC2)cc1-c1ccoc1] | 182 |
| 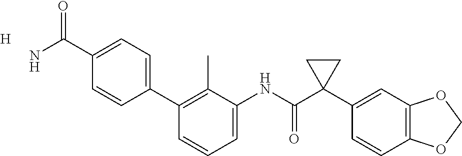 | 183 |
| 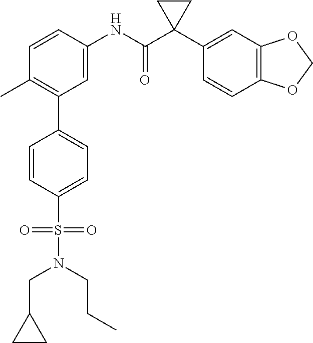 [SMILES: CCCN(CC1CC1)S(=O)(=O)c1ccc(-c2cc(NC(=O)C3(c4ccc5c(c4)OCO5)CC3)ccc2C)cc1] | 184 |
| 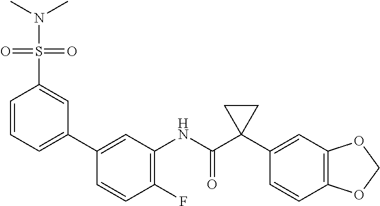 [SMILES: CN(C)S(=O)(=O)c1cccc(-c2ccc(F)c(NC(=O)C3(c4ccc5c(c4)OCO5)CC3)c2)c1] | 185 |
| 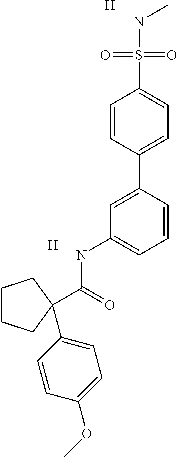 | 186 |
| 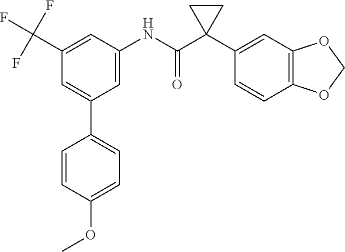 [SMILES: COc1ccc(-c2cc(NC(=O)C3(c4ccc5c(c4)OCO5)CC3)cc(C(F)(F)F)c2)cc1] | 187 |
| 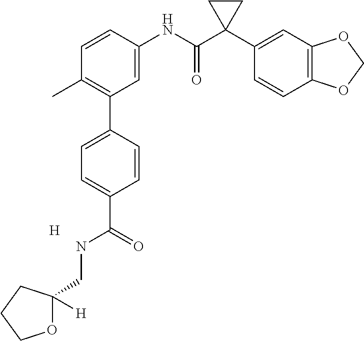 | 188 |
| 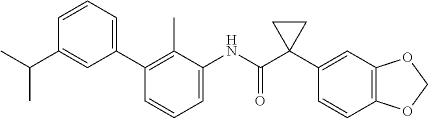 [SMILES: Cc1c(NC(=O)C2(c3ccc4c(c3)OCO4)CC2)cccc1-c1cccc(C(C)C)c1] | 189 |
| 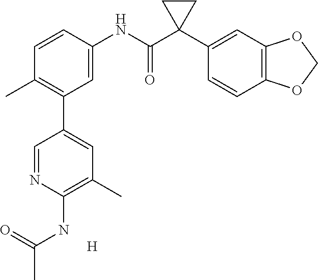 | 190 |
| 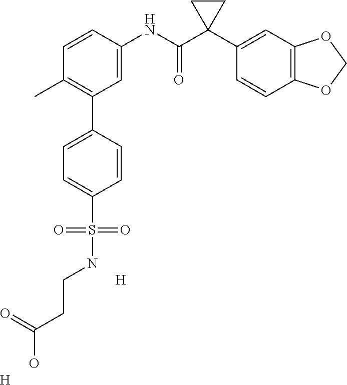 | 191 |
| 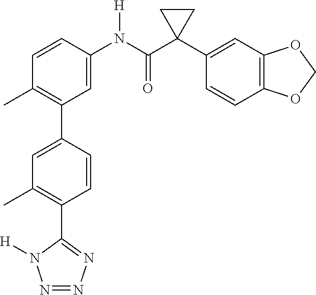 [SMILES: Cc1ccc(NC(=O)C2(c3ccc4c(c3)OCO4)CC2)cc1-c1ccc(-c2nnn[nH]2)c(C)c1] | 192 |
| 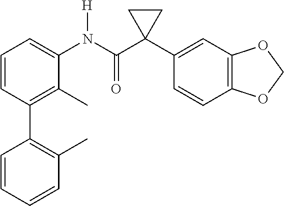 [SMILES: Cc1ccccc1-c1cccc(NC(=O)C2(c3ccc4c(c3)OCO4)CC2)c1C] | 193 |
| 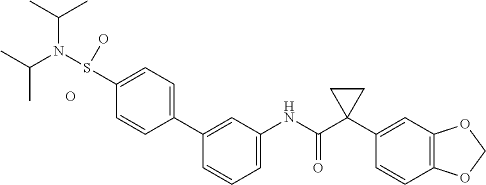 | 194 |
| 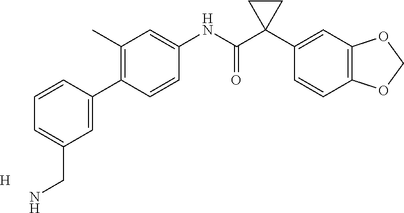 | 195 |
| 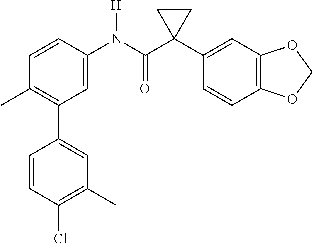 [SMILES: Cc1cc(-c2cc(NC(=O)C3(c4ccc5c(c4)OCO5)CC3)ccc2C)ccc1Cl] | 196 |
| 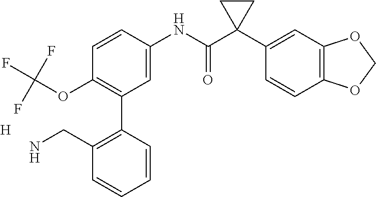 | 197 |
| 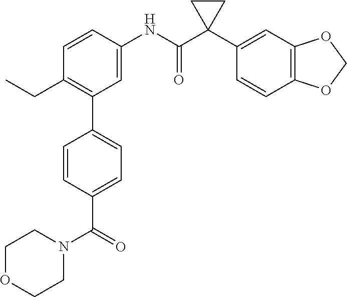 [SMILES: CCc1ccc(NC(=O)C2(c3ccc4c(c3)OCO4)CC2)cc1-c1ccc(C(=O)N2CCOCC2)cc1] | 198 |
| 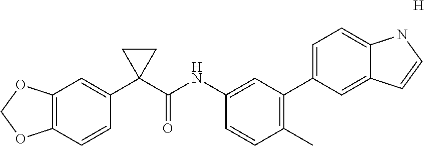 | 199 |
| 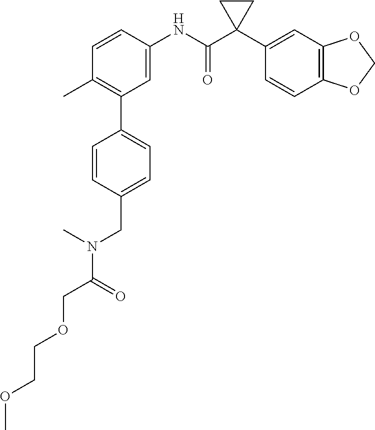 [SMILES: COCCOCC(=O)N(C)Cc1ccc(-c2cc(NC(=O)C3(c4ccc5c(c4)OCO5)CC3)ccc2C)cc1] | 200 |
| 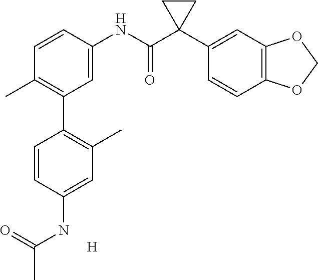 | 201 |
| 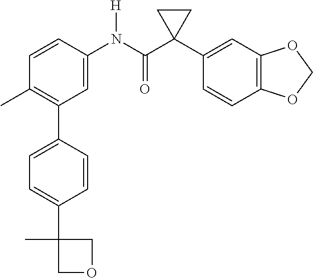 [SMILES: Cc1ccc(NC(=O)C2(c3ccc4c(c3)OCO4)CC2)cc1-c1ccc(C2(C)COC2)cc1] | 202 |
| 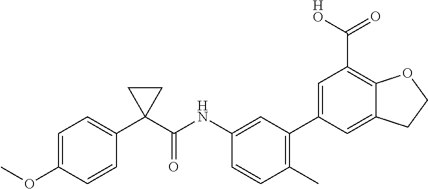 [SMILES: COc1ccc(C2(C(=O)Nc3ccc(C)c(-c4cc5c(c(C(=O)O)c4)OCC5)c3)CC2)cc1] | 203 |
| 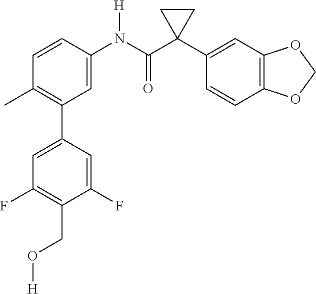 [SMILES: Cc1ccc(NC(=O)C2(c3ccc4c(c3)OCO4)CC2)cc1-c1cc(F)c(CO)c(F)c1] | 204 |
| 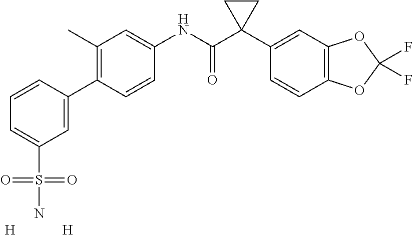 | 205 |
| 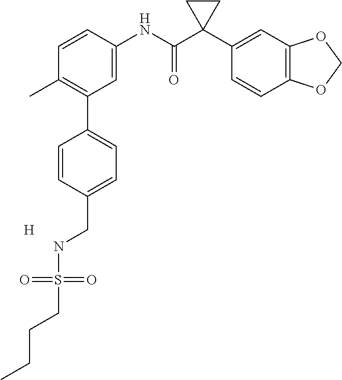 | 206 |
| 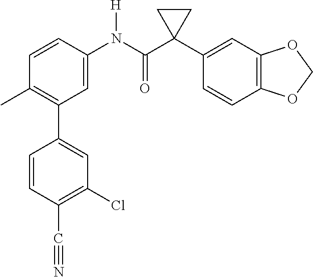 [SMILES: Cc1ccc(NC(=O)C2(c3ccc4c(c3)OCO4)CC2)cc1-c1ccc(C#N)c(Cl)c1] | 207 |
| 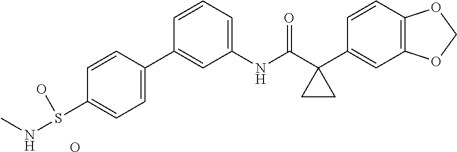 | 208 |
| 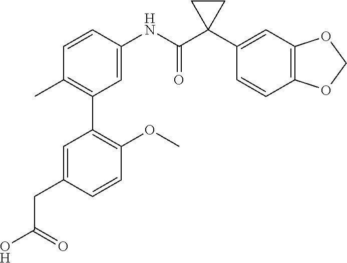 [SMILES: COc1ccc(CC(=O)O)cc1-c1cc(NC(=O)C2(c3ccc4c(c3)OCO4)CC2)ccc1C] | 209 |
| 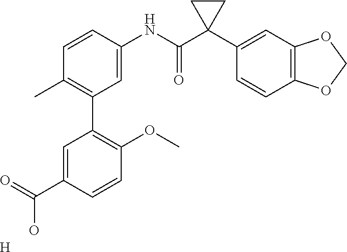 | 210 |
| 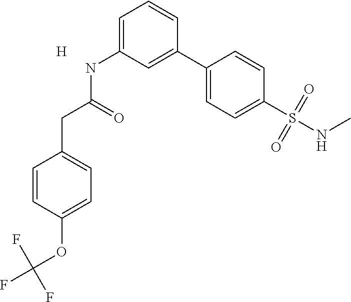 | 211 |
| 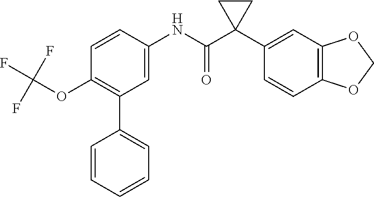 [SMILES: O=C(Nc1ccc(OC(F)(F)F)c(-c2ccccc2)c1)C1(c2ccc3c(c2)OCO3)CC1] | 212 |
| 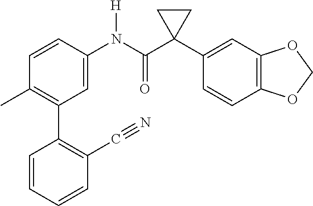 [SMILES: Cc1ccc(NC(=O)C2(c3ccc4c(c3)OCO4)CC2)cc1-c1ccccc1C#N] | 213 |
| 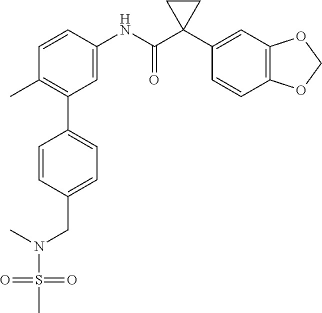 [SMILES: Cc1ccc(NC(=O)C2(c3ccc4c(c3)OCO4)CC2)cc1-c1ccc(CN(C)S(C)(=O)=O)cc1] | 214 |
| 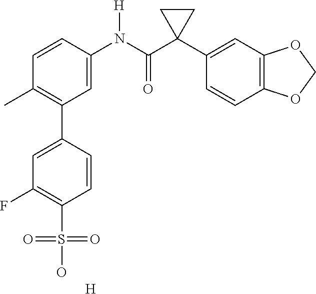 | 215 |
| 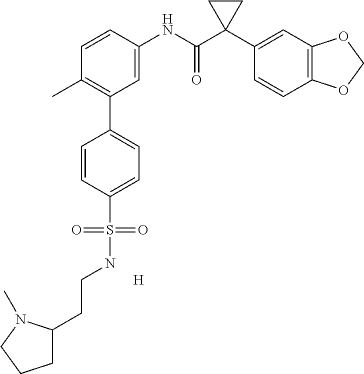 | 216 |
| 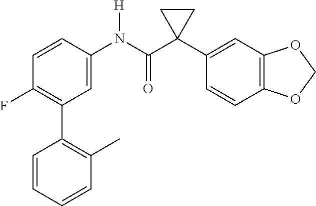 [SMILES: Cc1ccccc1-c1cc(NC(=O)C2(c3ccc4c(c3)OCO4)CC2)ccc1F] | 217 |
| 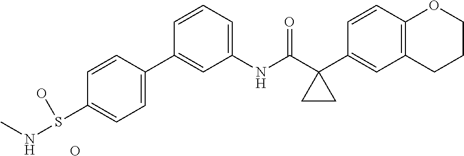 | 218 |
| 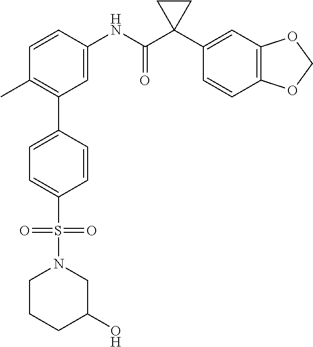 [SMILES: Cc1ccc(NC(=O)C2(c3ccc4c(c3)OCO4)CC2)cc1-c1ccc(S(=O)(=O)N2CCCC(O)C2)cc1] | 219 |
| 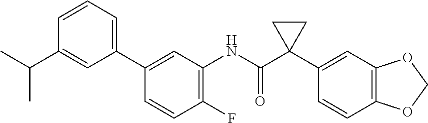 [SMILES: CC(C)c1cccc(-c2ccc(F)c(NC(=O)C3(c4ccc5c(c4)OCO5)CC3)c2)c1] | 220 |
| 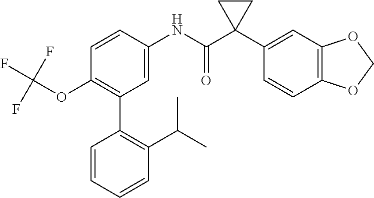 [SMILES: CC(C)c1ccccc1-c1cc(NC(=O)C2(c3ccc4c(c3)OCO4)CC2)ccc1OC(F)(F)F] | 221 |
| 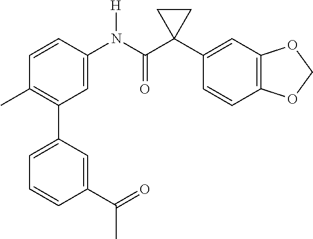 [SMILES: CC(=O)c1cccc(-c2cc(NC(=O)C3(c4ccc5c(c4)OCO5)CC3)ccc2C)c1] | 222 |
| 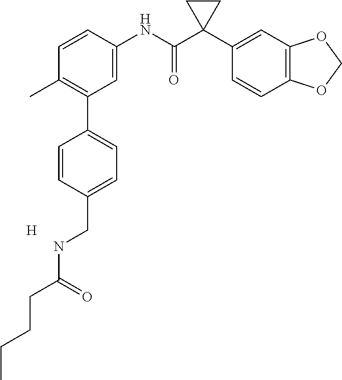 | 223 |
| 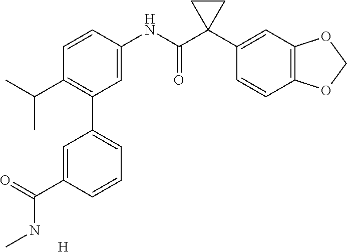 | 224 |
| 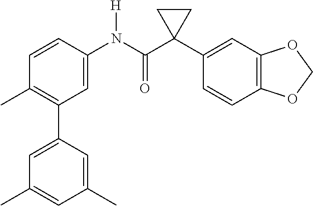 [SMILES: Cc1cc(C)cc(-c2cc(NC(=O)C3(c4ccc5c(c4)OCO5)CC3)ccc2C)c1] | 225 |
| 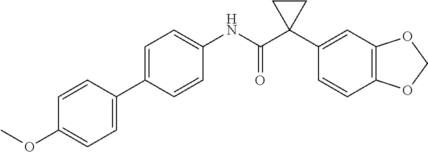 [SMILES: COc1ccc(-c2ccc(NC(=O)C3(c4ccc5c(c4)OCO5)CC3)cc2)cc1] | 226 |
| 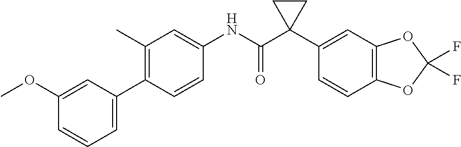 [SMILES: COc1cccc(-c2ccc(NC(=O)C3(c4ccc5c(c4)OC(F)(F)O5)CC3)cc2C)c1] | 227 |
| 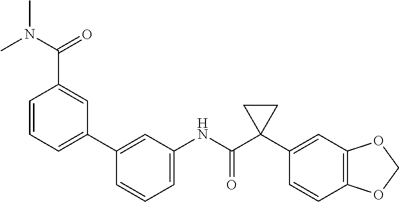 [SMILES: CN(C)C(=O)c1cccc(-c2cccc(NC(=O)C3(c4ccc5c(c4)OCO5)CC3)c2)c1] | 228 |
| 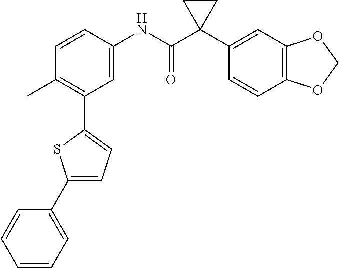 [SMILES: Cc1ccc(NC(=O)C2(c3ccc4c(c3)OCO4)CC2)cc1-c1ccc(-c2ccccc2)s1] | 229 |
| 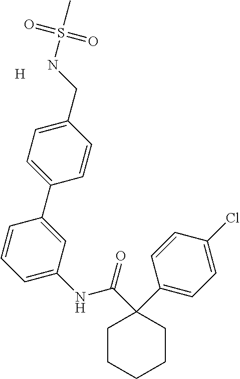 | 230 |
| 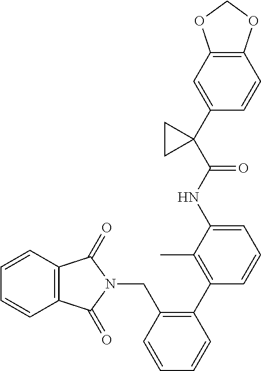 [SMILES: Cc1c(NC(=O)C2(c3ccc4c(c3)OCO4)CC2)cccc1-c1ccccc1CN1C(=O)c2ccccc2C1=O] | 231 |
| 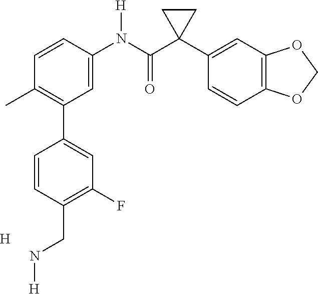 | 232 |
| 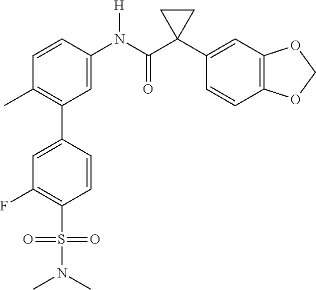 [SMILES: Cc1ccc(NC(=O)C2(c3ccc4c(c3)OCO4)CC2)cc1-c1ccc(S(=O)(=O)N(C)C)c(F)c1] | 233 |
| 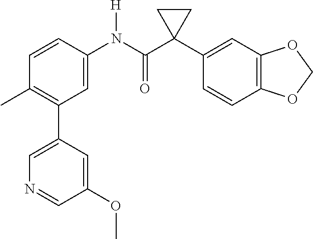 [SMILES: COc1cncc(-c2cc(NC(=O)C3(c4ccc5c(c4)OCO5)CC3)ccc2C)c1] | 234 |
| 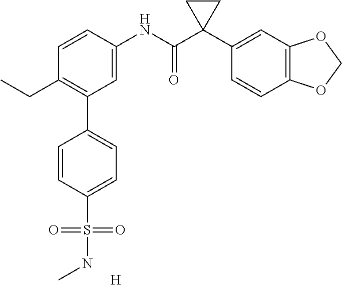 | 235 |
| 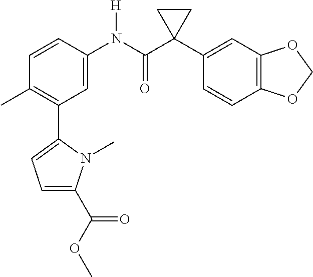 [SMILES: COC(=O)c1ccc(-c2cc(NC(=O)C3(c4ccc5c(c4)OCO5)CC3)ccc2C)n1C] | 236 |
| 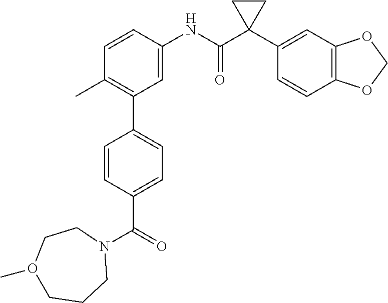 | 237 |
| 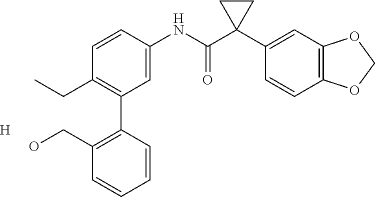 | 238 |
| 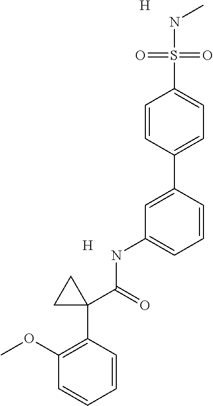 | 239 |
| 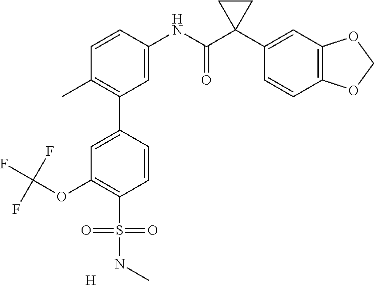 | 240 |
| 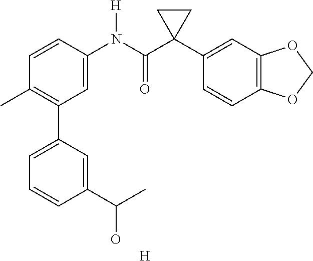 | 241 |
| 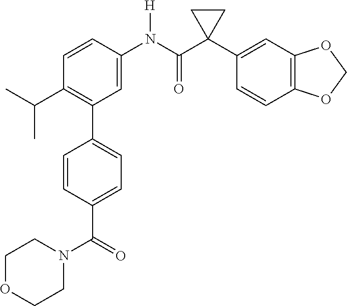 [SMILES: CC(C)c1ccc(NC(=O)C2(c3ccc4c(c3)OCO4)CC2)cc1-c1ccc(C(=O)N2CCOCC2)cc1] | 242 |
| 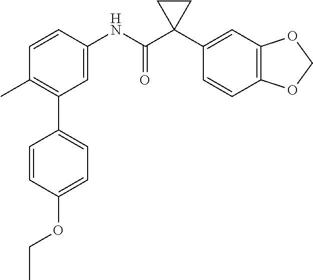 [SMILES: CCOc1ccc(-c2cc(NC(=O)C3(c4ccc5c(c4)OCO5)CC3)ccc2C)cc1] | 243 |
| 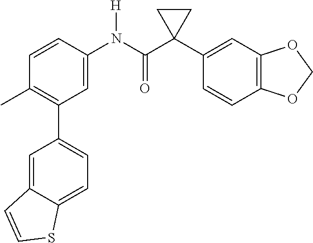 [SMILES: Cc1ccc(NC(=O)C2(c3ccc4c(c3)OCO4)CC2)cc1-c1ccc2sccc2c1] | 244 |
| 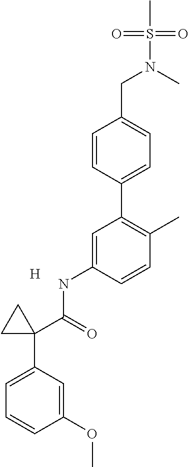 | 245 |
| 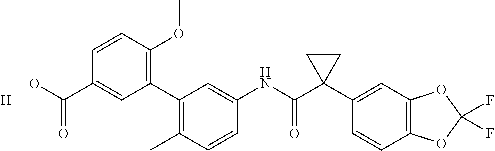 | 246 |
| 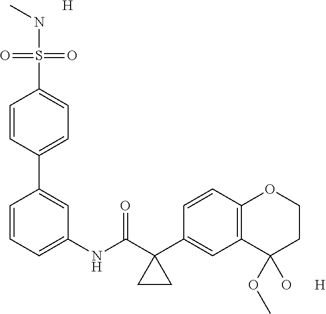 | 247 |
| 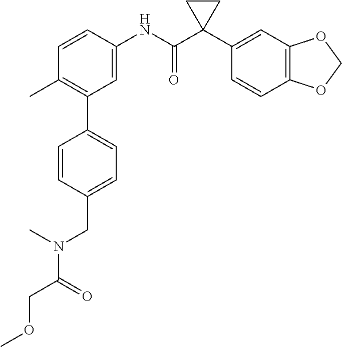 [SMILES: COCC(=O)N(C)Cc1ccc(-c2cc(NC(=O)C3(c4ccc5c(c4)OCO5)CC3)ccc2C)cc1] | 248 |
| 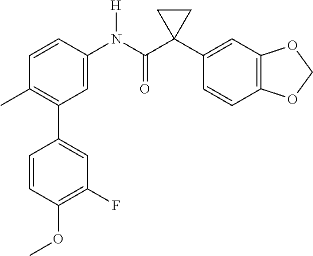 [SMILES: COc1ccc(-c2cc(NC(=O)C3(c4ccc5c(c4)OCO5)CC3)ccc2C)cc1F] | 249 |
| 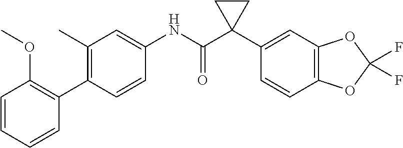 [SMILES: COc1ccccc1-c1ccc(NC(=O)C2(c3ccc4c(c3)OC(F)(F)O4)CC2)cc1C] | 250 |
| 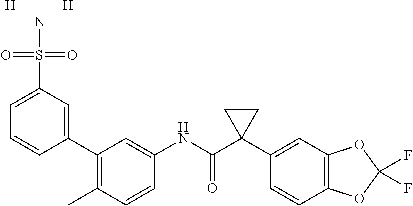 | 251 |
| 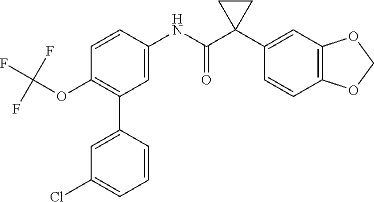 [SMILES: O=C(Nc1ccc(OC(F)(F)F)c(-c2cccc(Cl)c2)c1)C1(c2ccc3c(c2)OCO3)CC1] | 252 |
| 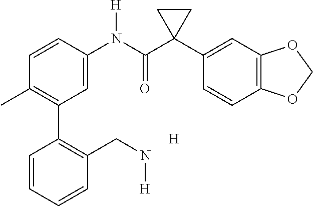 | 253 |
| 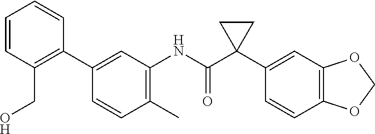 [SMILES: Cc1ccc(-c2ccccc2CO)cc1NC(=O)C1(c2ccc3c(c2)OCO3)CC1] | 254 |
| 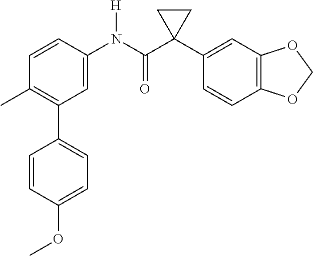 [SMILES: COc1ccc(-c2cc(NC(=O)C3(c4ccc5c(c4)OCO5)CC3)ccc2C)cc1] | 255 |
| 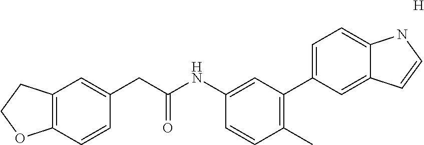 | 256 |
| 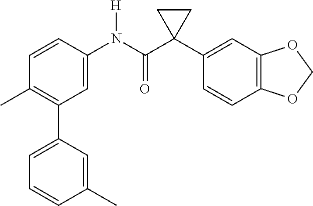 [SMILES: Cc1cccc(-c2cc(NC(=O)C3(c4ccc5c(c4)OCO5)CC3)ccc2C)c1] | 257 |
| 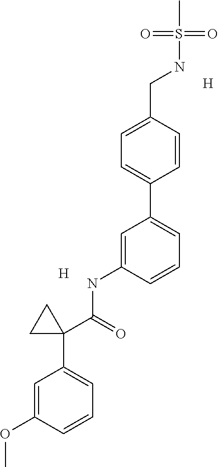 | 258 |
| 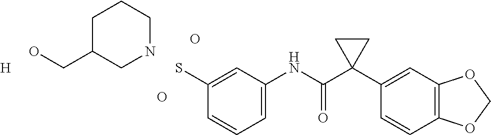 | 259 |
| 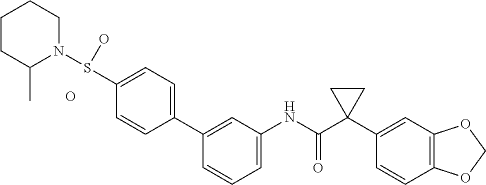 | 260 |
| 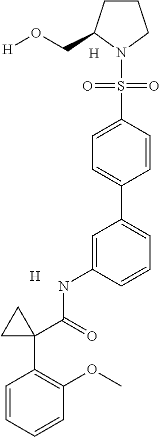 | 261 |
| 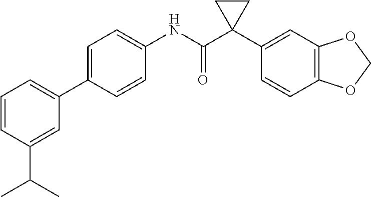 [SMILES: CC(C)c1cccc(-c2ccc(NC(=O)C3(c4ccc5c(c4)OCO5)CC3)cc2)c1] | 262 |
| 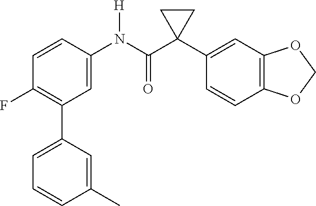 [SMILES: Cc1cccc(-c2cc(NC(=O)C3(c4ccc5c(c4)OCO5)CC3)ccc2F)c1] | 263 |
| 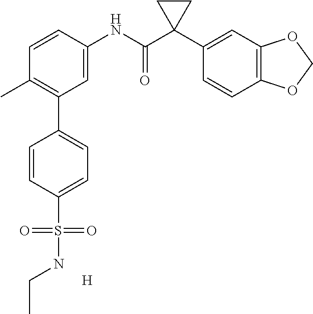 | 264 |
| 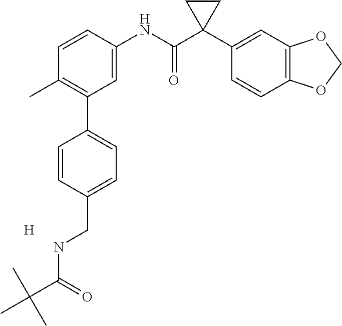 | 265 |
| 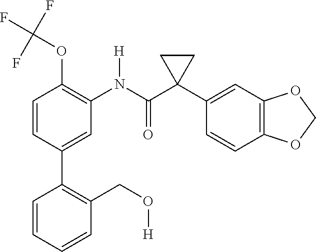 [SMILES: O=C(Nc1cc(-c2ccccc2CO)ccc1OC(F)(F)F)C1(c2ccc3c(c2)OCO3)CC1] | 266 |
| 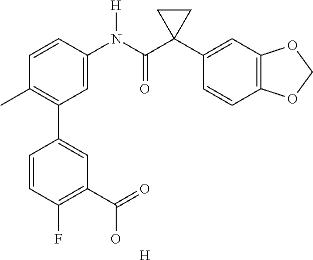 | 267 |
| 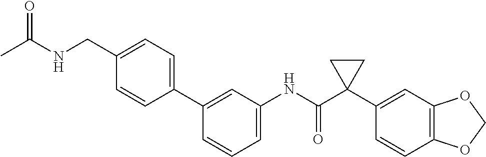 [SMILES: CC(=O)NCc1ccc(-c2cccc(NC(=O)C3(c4ccc5c(c4)OCO5)CC3)c2)cc1] | 268 |
| 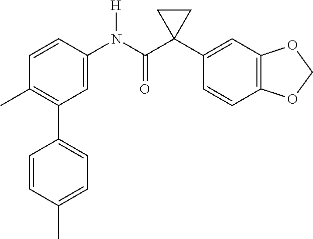 [SMILES: Cc1ccc(-c2cc(NC(=O)C3(c4ccc5c(c4)OCO5)CC3)ccc2C)cc1] | 269 |
| 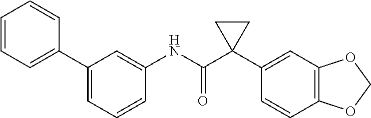 [SMILES: O=C(Nc1cccc(-c2ccccc2)c1)C1(c2ccc3c(c2)OCO3)CC1] | 270 |
| 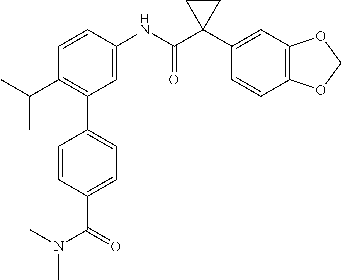 [SMILES: CC(C)c1ccc(NC(=O)C2(c3ccc4c(c3)OCO4)CC2)cc1-c1ccc(C(=O)N(C)C)cc1] | 271 |
| 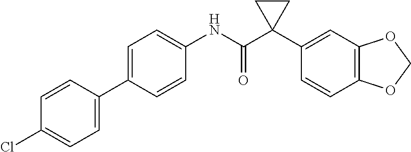 [SMILES: O=C(Nc1ccc(-c2ccc(Cl)cc2)cc1)C1(c2ccc3c(c2)OCO3)CC1] | 272 |
| 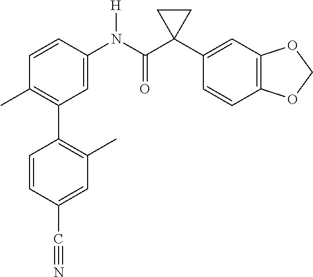 [SMILES: Cc1cc(C#N)ccc1-c1cc(NC(=O)C2(c3ccc4c(c3)OCO4)CC2)ccc1C] | 273 |
| 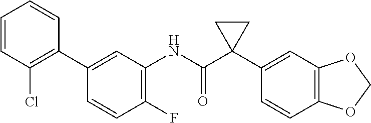 [SMILES: O=C(Nc1cc(-c2ccccc2Cl)ccc1F)C1(c2ccc3c(c2)OCO3)CC1] | 274 |
| 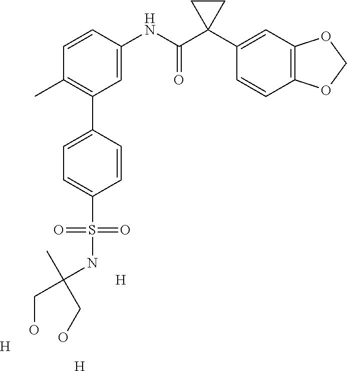 | 275 |
| 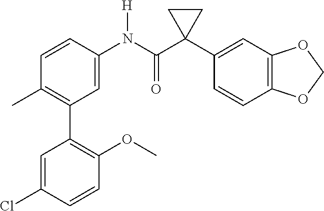 [SMILES: COc1ccc(Cl)cc1-c1cc(NC(=O)C2(c3ccc4c(c3)OCO4)CC2)ccc1C] | 276 |
| 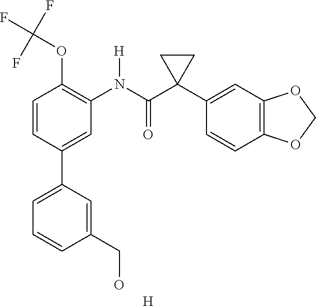 | 277 |
| 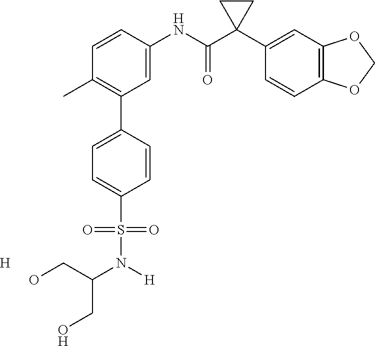 | 278 |
| 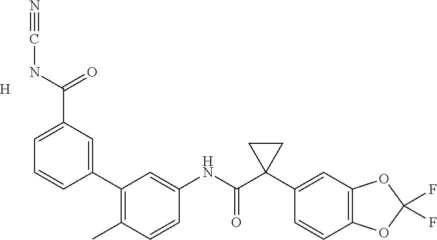 | 279 |
| 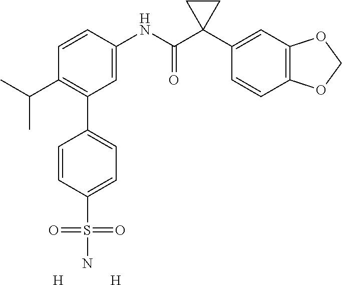 | 280 |
| 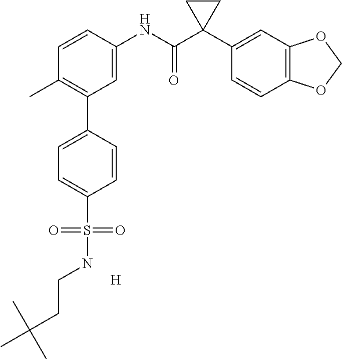 | 281 |
| 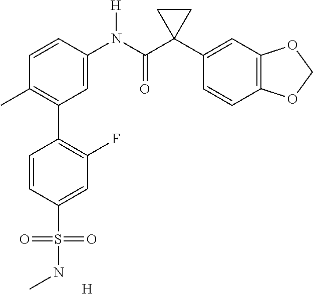 | 282 |
| 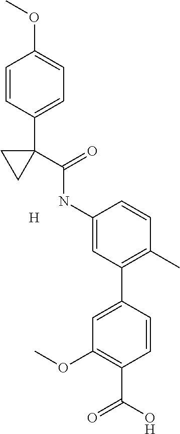 | 283 |
| 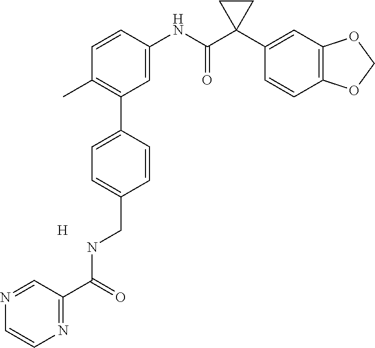 | 284 |
| 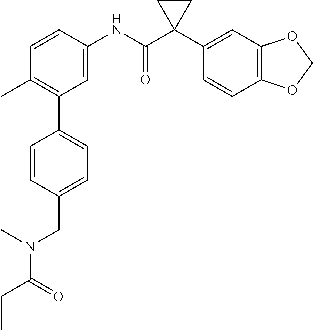 [SMILES: CCC(=O)N(C)Cc1ccc(-c2cc(NC(=O)C3(c4ccc5c(c4)OCO5)CC3)ccc2C)cc1] | 285 |
| 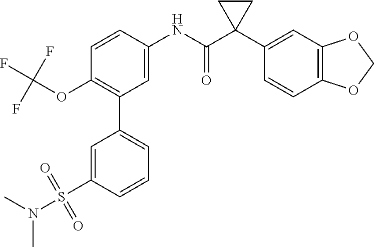 [SMILES: CN(C)S(=O)(=O)c1cccc(-c2cc(NC(=O)C3(c4ccc5c(c4)OCO5)CC3)ccc2OC(F)(F)F)c1] | 286 |
| 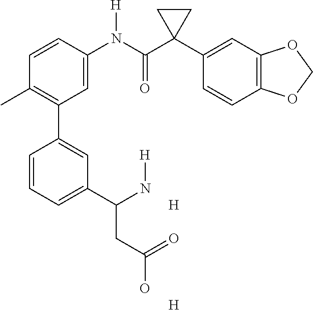 | 287 |
| 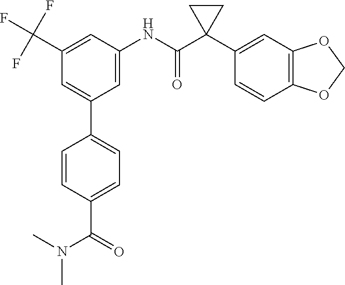 [SMILES: CN(C)C(=O)c1ccc(-c2cc(NC(=O)C3(c4ccc5c(c4)OCO5)CC3)cc(C(F)(F)F)c2)cc1] | 288 |
| 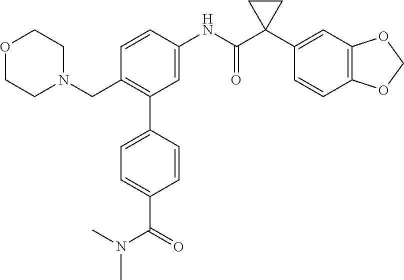 [SMILES: CN(C)C(=O)c1ccc(-c2cc(NC(=O)C3(c4ccc5c(c4)OCO5)CC3)ccc2CN2CCOCC2)cc1] | 289 |
| 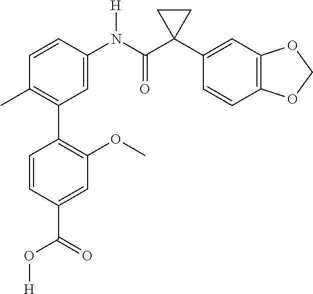 [SMILES: COc1cc(C(=O)O)ccc1-c1cc(NC(=O)C2(c3ccc4c(c3)OCO4)CC2)ccc1C] | 290 |
| 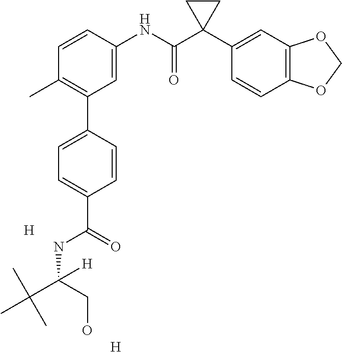 | 291 |
| 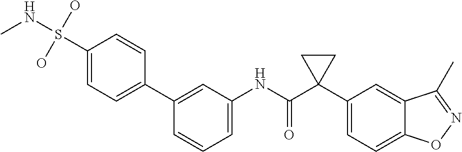 [SMILES: CNS(O)(O)c1ccc(-c2cccc(NC(=O)C3(c4ccc5onc(C)c5c4)CC3)c2)cc1] | 292 |
| 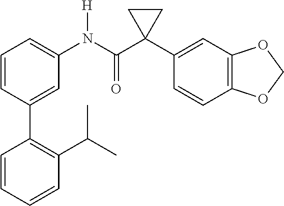 [SMILES: CC(C)c1ccccc1-c1cccc(NC(=O)C2(c3ccc4c(c3)OCO4)CC2)c1] | 293 |
| 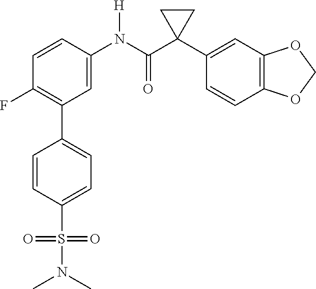 [SMILES: CN(C)S(=O)(=O)c1ccc(-c2cc(NC(=O)C3(c4ccc5c(c4)OCO5)CC3)ccc2F)cc1] | 294 |
| 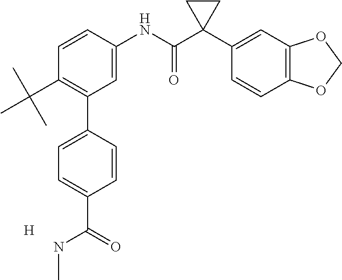 | 295 |
| 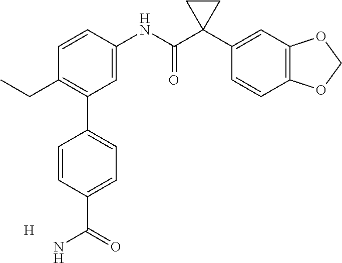 | 296 |
| 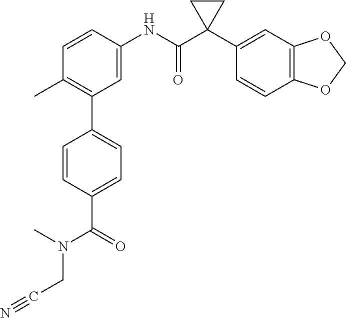 [SMILES: Cc1ccc(NC(=O)C2(c3ccc4c(c3)OCO4)CC2)cc1-c1ccc(C(=O)N(C)CC#N)cc1] | 297 |
| 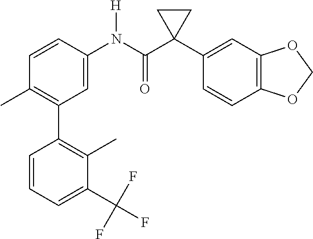 [SMILES: Cc1ccc(NC(=O)C2(c3ccc4c(c3)OCO4)CC2)cc1-c1cccc(C(F)(F)F)c1C] | 298 |
| 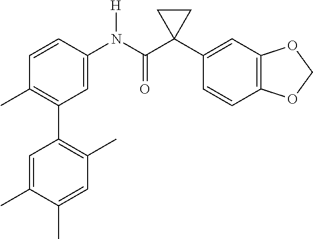 [SMILES: Cc1cc(C)c(-c2cc(NC(=O)C3(c4ccc5c(c4)OCO5)CC3)ccc2C)cc1C] | 299 |
| 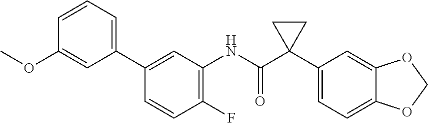 [SMILES: COc1cccc(-c2ccc(F)c(NC(=O)C3(c4ccc5c(c4)OCO5)CC3)c2)c1] | 300 |
| 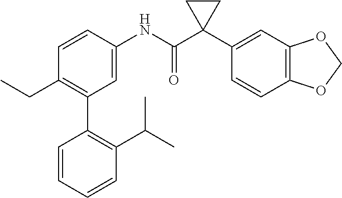 [SMILES: CCc1ccc(NC(=O)C2(c3ccc4c(c3)OCO4)CC2)cc1-c1ccccc1C(C)C] | 301 |
| 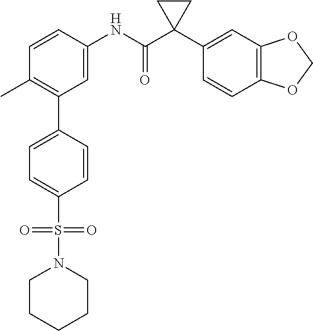 [SMILES: Cc1ccc(NC(=O)C2(c3ccc4c(c3)OCO4)CC2)cc1-c1ccc(S(=O)(=O)N2CCCCC2)cc1] | 302 |
| 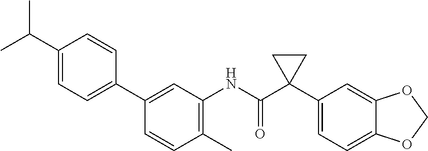 [SMILES: Cc1ccc(-c2ccc(C(C)C)cc2)cc1NC(=O)C1(c2ccc3c(c2)OCO3)CC1] | 303 |
| 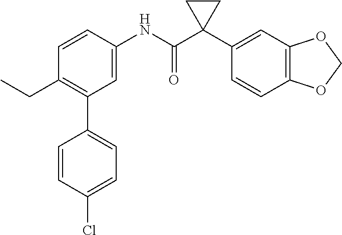 [SMILES: CCc1ccc(NC(=O)C2(c3ccc4c(c3)OCO4)CC2)cc1-c1ccc(Cl)cc1] | 304 |
| 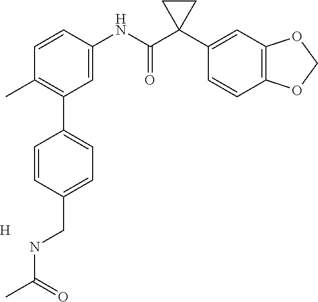 | 305 |
| 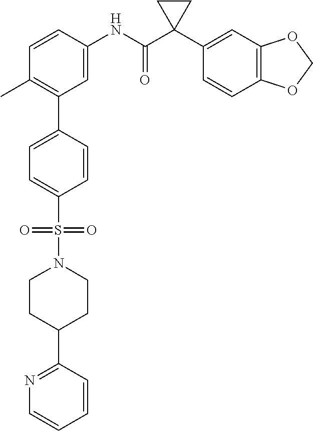 [SMILES: Cc1ccc(NC(=O)C2(c3ccc4c(c3)OCO4)CC2)cc1-c1ccc(S(=O)(=O)N2CCC(c3ccccn3)CC2)cc1] | 306 |
| 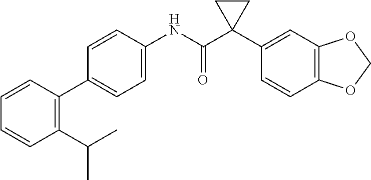 [SMILES: CC(C)c1ccccc1-c1ccc(NC(=O)C2(c3ccc4c(c3)OCO4)CC2)cc1] | 307 |
| 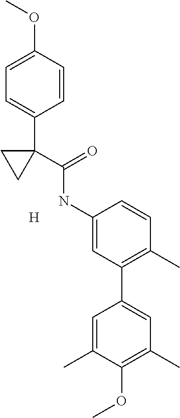 | 308 |
| 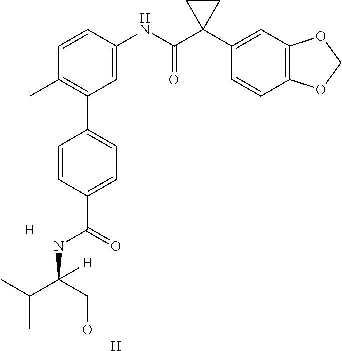 | 309 |
| 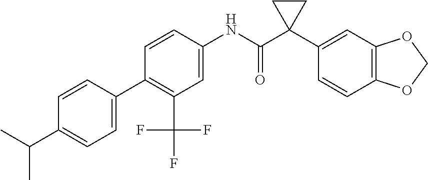 [SMILES: CC(C)c1ccc(-c2ccc(NC(=O)C3(c4ccc5c(c4)OCO5)CC3)cc2C(F)(F)F)cc1] | 310 |
| 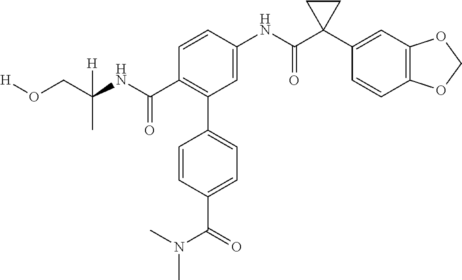 [SMILES: C[C@@H](CO)NC(=O)c1ccc(NC(=O)C2(c3ccc4c(c3)OCO4)CC2)cc1-c1ccc(C(=O)N(C)C)cc1] | 311 |
| 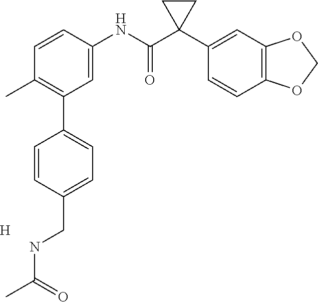 | 312 |
| 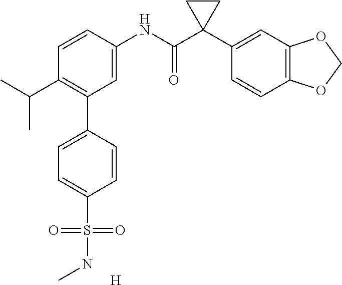 | 313 |
| 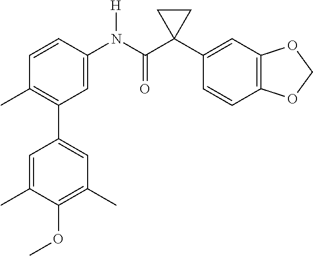 [SMILES: COc1c(C)cc(-c2cc(NC(=O)C3(c4ccc5c(c4)OCO5)CC3)ccc2C)cc1C] | 314 |
| 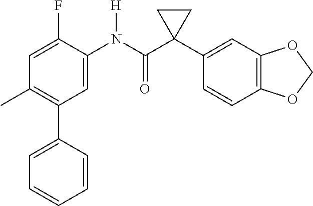 [SMILES: Cc1cc(F)c(NC(=O)C2(c3ccc4c(c3)OCO4)CC2)cc1-c1ccccc1] | 315 |
| 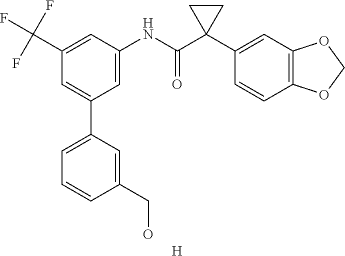 | 316 |
| 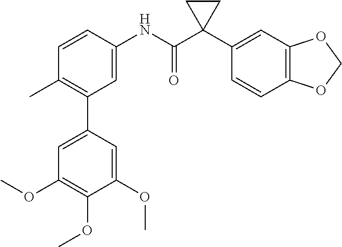 [SMILES: COc1cc(-c2cc(NC(=O)C3(c4ccc5c(c4)OCO5)CC3)ccc2C)cc(OC)c1OC] | 317 |
| 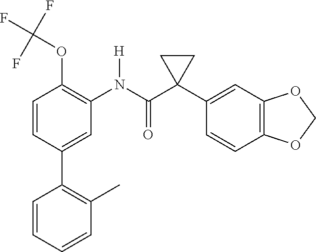 [SMILES: Cc1ccccc1-c1ccc(OC(F)(F)F)c(NC(=O)C2(c3ccc4c(c3)OCO4)CC2)c1] | 318 |
| 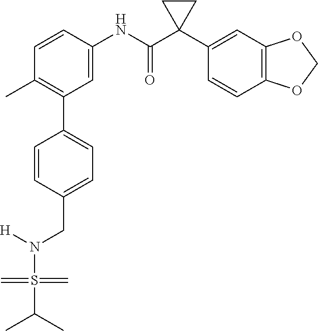 [SMILES: C=S(=C)(NCc1ccc(-c2cc(NC(=O)C3(c4ccc5c(c4)OCO5)CC3)ccc2C)cc1)C(C)C] | 319 |
| 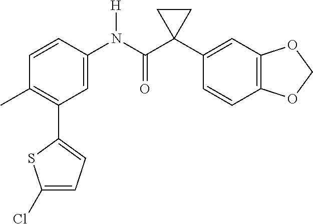 [SMILES: Cc1ccc(NC(=O)C2(c3ccc4c(c3)OCO4)CC2)cc1-c1ccc(Cl)s1] | 320 |
| 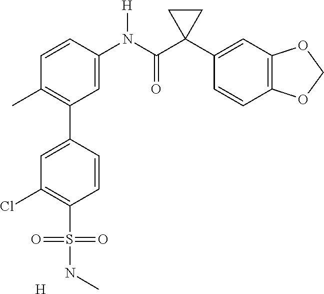 | 321 |
| 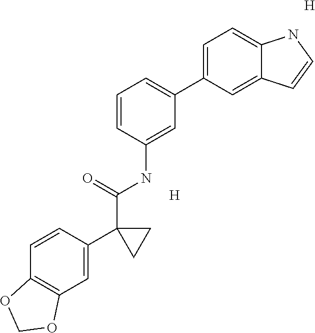 | 322 |
| 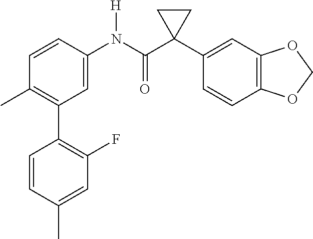 [SMILES: Cc1ccc(-c2cc(NC(=O)C3(c4ccc5c(c4)OCO5)CC3)ccc2C)c(F)c1] | 323 |
| 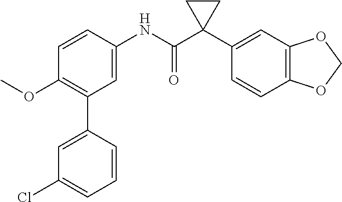 [SMILES: COc1ccc(NC(=O)C2(c3ccc4c(c3)OCO4)CC2)cc1-c1cccc(Cl)c1] | 324 |
| 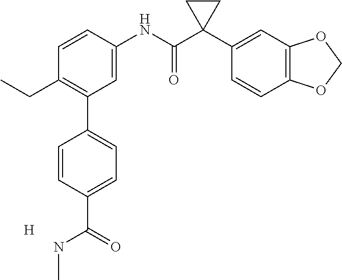 | 325 |
| 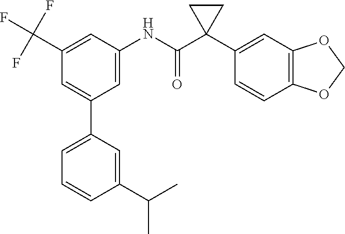 [SMILES: CC(C)c1cccc(-c2cc(NC(=O)C3(c4ccc5c(c4)OCO5)CC3)cc(C(F)(F)F)c2)c1] | 326 |
| 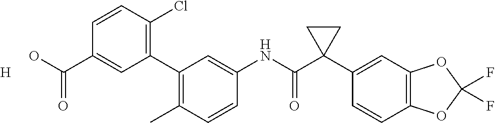 | 327 |
| 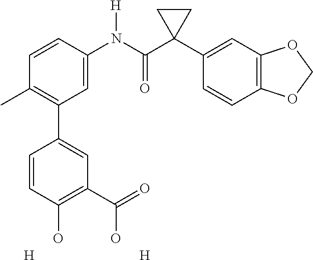 | 328 |
| 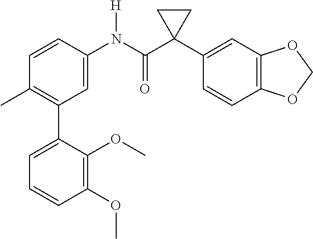 [SMILES: COc1cccc(-c2cc(NC(=O)C3(c4ccc5c(c4)OCO5)CC3)ccc2C)c1OC] | 329 |
| 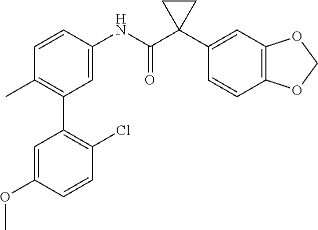 [SMILES: COc1ccc(Cl)c(-c2cc(NC(=O)C3(c4ccc5c(c4)OCO5)CC3)ccc2C)c1] | 330 |
| 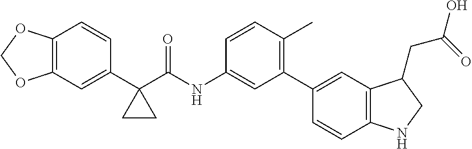 [SMILES: Cc1ccc(NC(=O)C2(c3ccc4c(c3)OCO4)CC2)cc1-c1ccc2c(c1)C(CC(=O)O)CN2] | 331 |
| 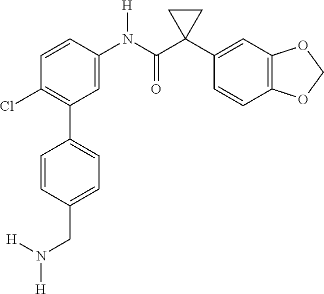 [SMILES: NCc1ccc(-c2cc(NC(=O)C3(c4ccc5c(c4)OCO5)CC3)ccc2Cl)cc1] | 332 |
| 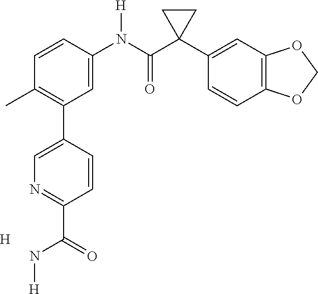 | 333 |
| 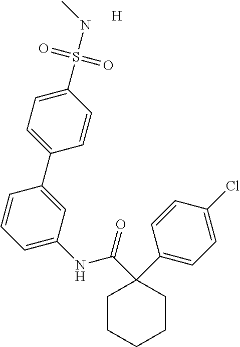 | 334 |
| 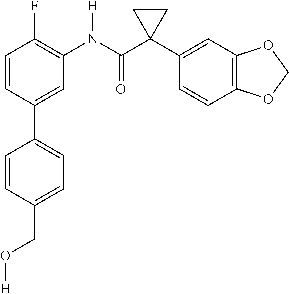 [SMILES: O=C(Nc1cc(-c2ccc(CO)cc2)ccc1F)C1(c2ccc3c(c2)OCO3)CC1] | 335 |
| 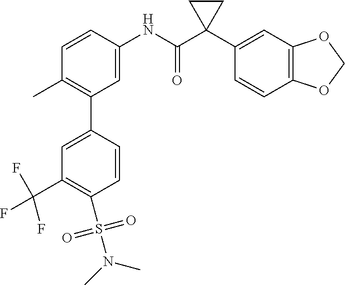 [SMILES: Cc1ccc(NC(=O)C2(c3ccc4c(c3)OCO4)CC2)cc1-c1ccc(S(=O)(=O)N(C)C)c(C(F)(F)F)c1] | 336 |
| 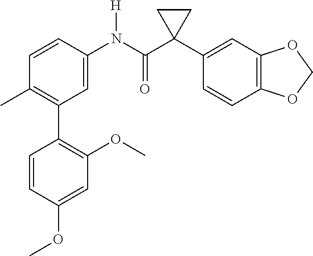 [SMILES: COc1ccc(-c2cc(NC(=O)C3(c4ccc5c(c4)OCO5)CC3)ccc2C)c(OC)c1] | 337 |
| 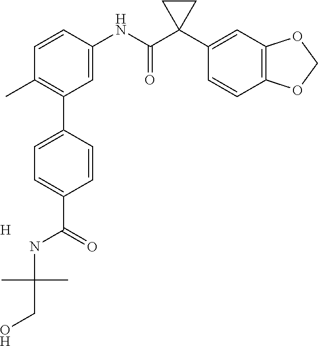 | 338 |
| 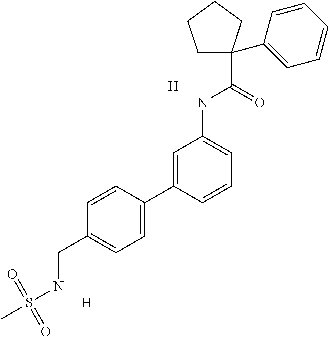 | 339 |
| 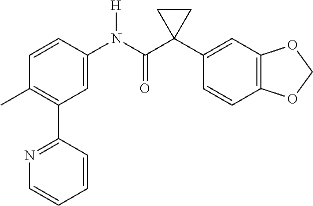 [SMILES: Cc1ccc(NC(=O)C2(c3ccc4c(c3)OCO4)CC2)cc1-c1ccccn1] | 340 |
| 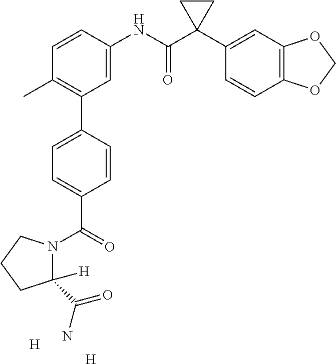 | 341 |
| 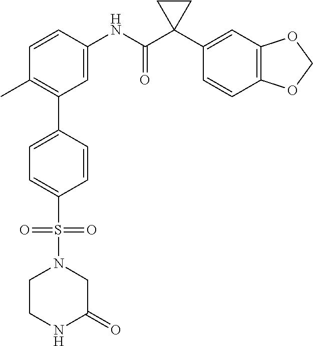 [SMILES: Cc1ccc(NC(=O)C2(c3ccc4c(c3)OCO4)CC2)cc1-c1ccc(S(=O)(=O)N2CCNC(=O)C2)cc1] | 342 |
| 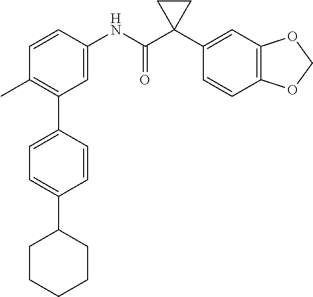 [SMILES: Cc1ccc(NC(=O)C2(c3ccc4c(c3)OCO4)CC2)cc1-c1ccc(C2CCCCC2)cc1] | 343 |
| 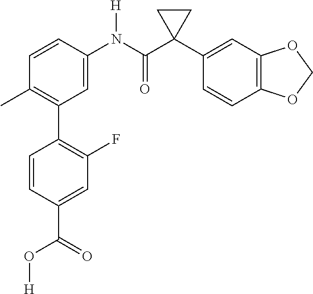 [SMILES: Cc1ccc(NC(=O)C2(c3ccc4c(c3)OCO4)CC2)cc1-c1ccc(C(=O)O)cc1F] | 344 |
| 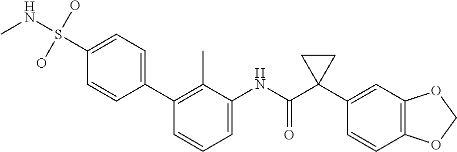 [SMILES: CNS(O)(O)c1ccc(-c2cccc(NC(=O)C3(c4ccc5c(c4)OCO5)CC3)c2C)cc1] | 345 |
| 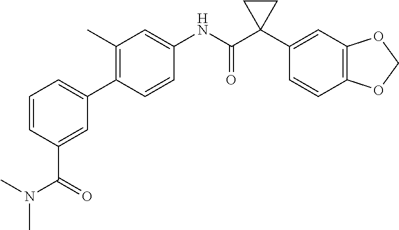 [SMILES: Cc1cc(NC(=O)C2(c3ccc4c(c3)OCO4)CC2)ccc1-c1cccc(C(=O)N(C)C)c1] | 346 |
| 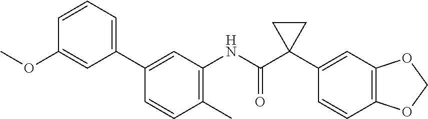 [SMILES: COc1cccc(-c2ccc(C)c(NC(=O)C3(c4ccc5c(c4)OCO5)CC3)c2)c1] | 347 |
| 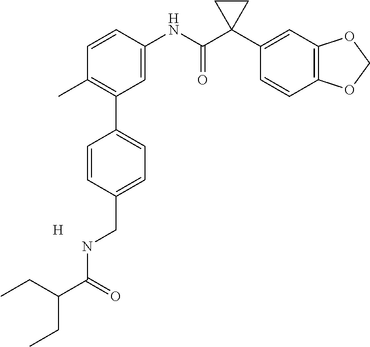 | 348 |
| 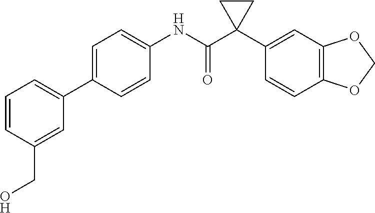 [SMILES: O=C(Nc1ccc(-c2cccc(CO)c2)cc1)C1(c2ccc3c(c2)OCO3)CC1] | 349 |
| 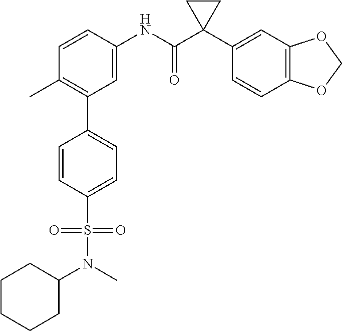 [SMILES: Cc1ccc(NC(=O)C2(c3ccc4c(c3)OCO4)CC2)cc1-c1ccc(S(=O)(=O)N(C)C2CCCCC2)cc1] | 350 |
| 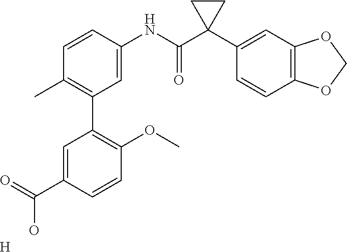 | 351 |
| 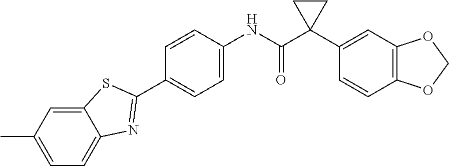 [SMILES: Cc1ccc2nc(-c3ccc(NC(=O)C4(c5ccc6c(c5)OCO6)CC4)cc3)sc2c1] | 352 |
| 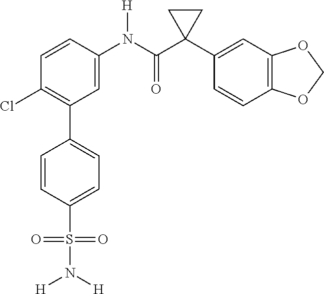 [SMILES: NS(=O)(=O)c1ccc(-c2cc(NC(=O)C3(c4ccc5c(c4)OCO5)CC3)ccc2Cl)cc1] | 353 |
| 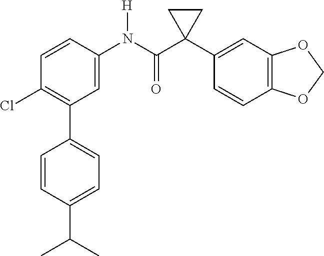 [SMILES: CC(C)c1ccc(-c2cc(NC(=O)C3(c4ccc5c(c4)OCO5)CC3)ccc2Cl)cc1] | 354 |
| 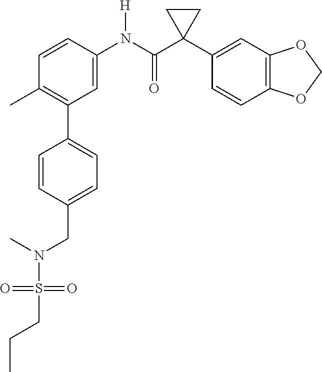 [SMILES: CCCS(=O)(=O)N(C)Cc1ccc(-c2cc(NC(=O)C3(c4ccc5c(c4)OCO5)CC3)ccc2C)cc1] | 355 |
| 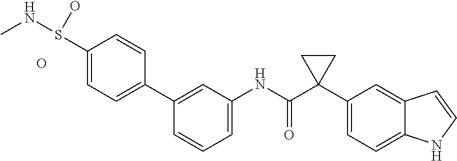 | 356 |
| 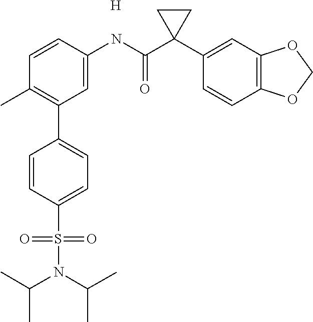 | 357 |
| 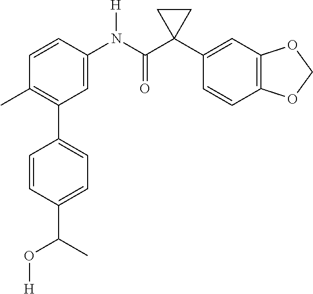 [SMILES: Cc1ccc(NC(=O)C2(c3ccc4c(c3)OCO4)CC2)cc1-c1ccc(C(C)O)cc1] | 358 |
| 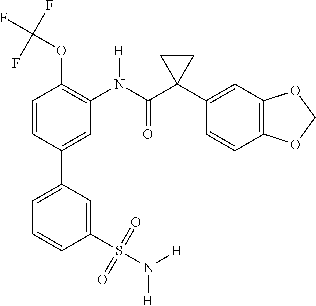 [SMILES: NS(=O)(=O)c1cccc(-c2ccc(OC(F)(F)F)c(NC(=O)C3(c4ccc5c(c4)OCO5)CC3)c2)c1] | 359 |
| 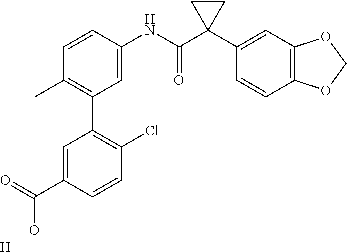 | 360 |
| 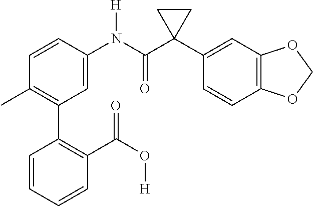 [SMILES: Cc1ccc(NC(=O)C2(c3ccc4c(c3)OCO4)CC2)cc1-c1ccccc1C(=O)O] | 361 |
| 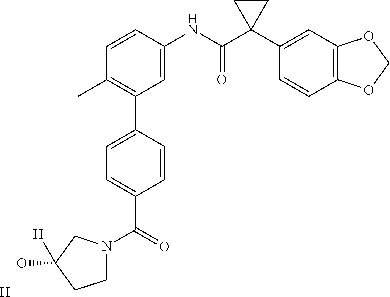 | 362 |
| 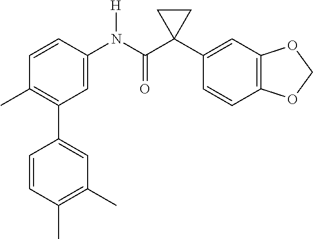 [SMILES: Cc1ccc(-c2cc(NC(=O)C3(c4ccc5c(c4)OCO5)CC3)ccc2C)cc1C] | 363 |
| 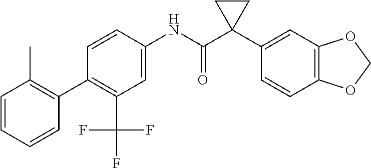 [SMILES: Cc1ccccc1-c1ccc(NC(=O)C2(c3ccc4c(c3)OCO4)CC2)cc1C(F)(F)F] | 364 |
| 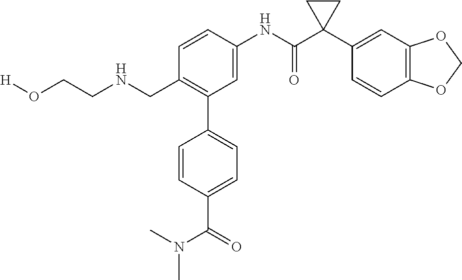 [SMILES: CN(C)C(=O)c1ccc(-c2cc(NC(=O)C3(c4ccc5c(c4)OCO5)CC3)ccc2CNCCO)cc1] | 365 |
| 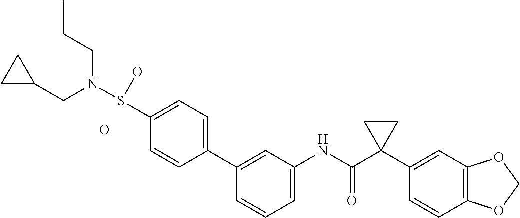 | 366 |
| 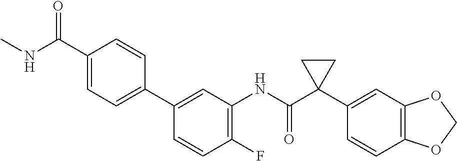 [SMILES: CNC(=O)c1ccc(-c2ccc(F)c(NC(=O)C3(c4ccc5c(c4)OCO5)CC3)c2)cc1] | 367 |
| 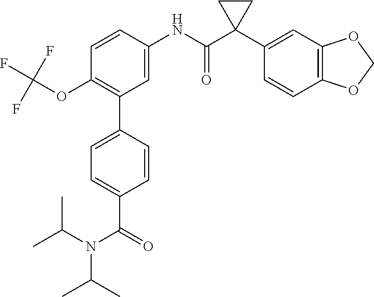 [SMILES: CC(C)N(C(=O)c1ccc(-c2cc(NC(=O)C3(c4ccc5c(c4)OCO5)CC3)ccc2OC(F)(F)F)cc1)C(C)C] | 368 |
| 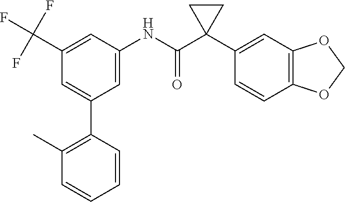 | 369 |
| 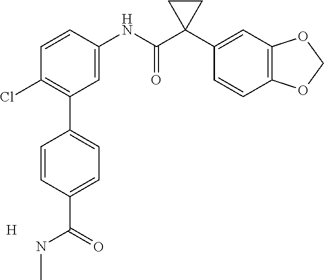 | 370 |
| 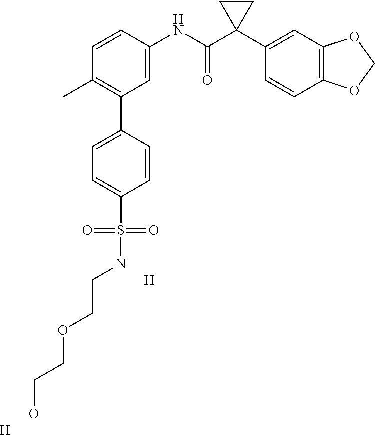 | 371 |
| 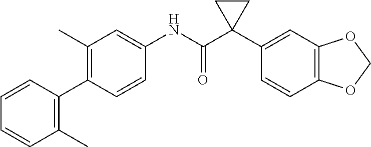 [SMILES: Cc1ccccc1-c1ccc(NC(=O)C2(c3ccc4c(c3)OCO4)CC2)cc1C] | 372 |
| 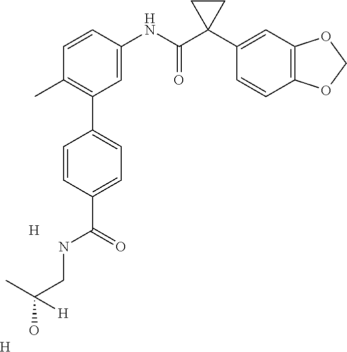 | 373 |
| 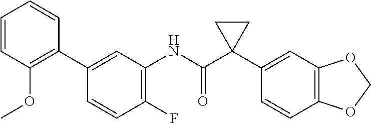 [SMILES: COc1ccccc1-c1ccc(F)c(NC(=O)C2(c3ccc4c(c3)OCO4)CC2)c1] | 374 |
| 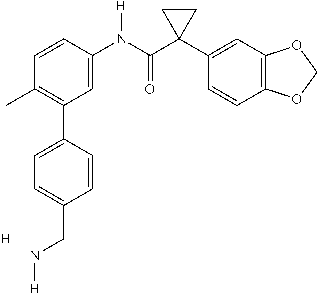 | 375 |
| 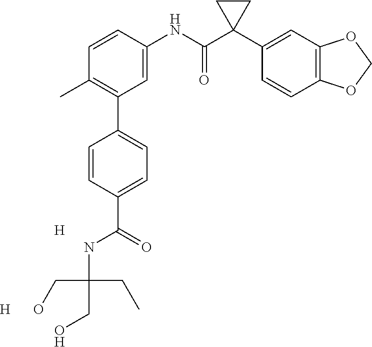 | 376 |
| 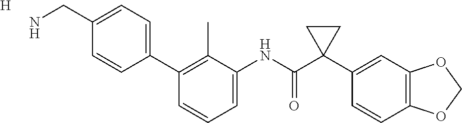 | 377 |
| 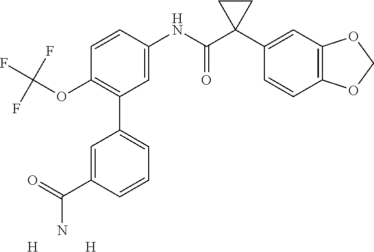 | 378 |
| 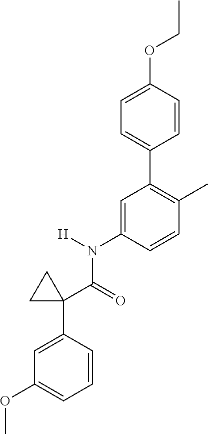 [SMILES: CCOc1ccc(-c2cc(NC(=O)C3(c4cccc(OC)c4)CC3)ccc2C)cc1] | 379 |
| 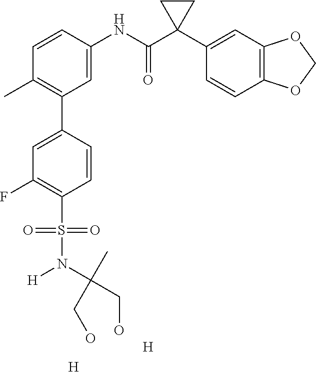 | 380 |
| 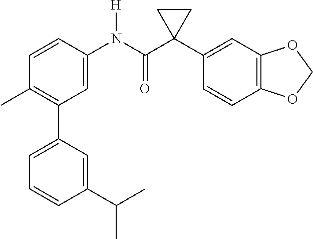 [SMILES: Cc1ccc(NC(=O)C2(c3ccc4c(c3)OCO4)CC2)cc1-c1cccc(C(C)C)c1] | 381 |
| 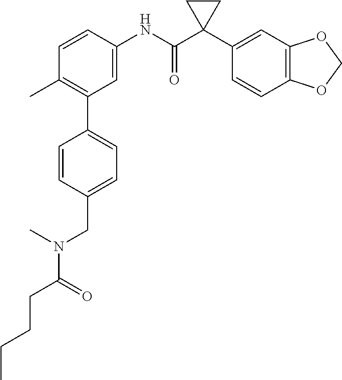 [SMILES: CCCCC(=O)N(C)Cc1ccc(-c2cc(NC(=O)C3(c4ccc5c(c4)OCO5)CC3)ccc2C)cc1] | 382 |
| 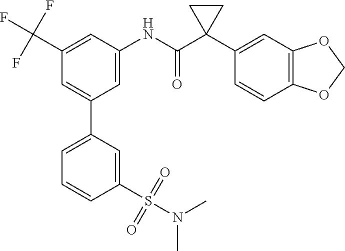 [SMILES: CN(C)S(=O)(=O)c1cccc(-c2cc(NC(=O)C3(c4ccc5c(c4)OCO5)CC3)cc(C(F)(F)F)c2)c1] | 383 |
| 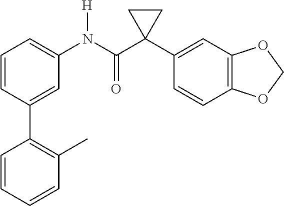 [SMILES: Cc1ccccc1-c1cccc(NC(=O)C2(c3ccc4c(c3)OCO4)CC2)c1] | 384 |
| 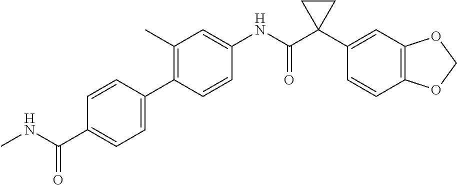 [SMILES: CNC(=O)c1ccc(-c2ccc(NC(=O)C3(c4ccc5c(c4)OCO5)CC3)cc2C)cc1] | 385 |
| 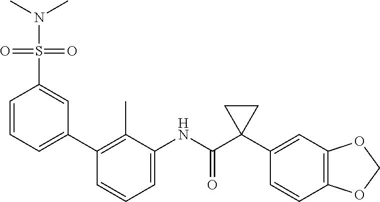 [SMILES: Cc1c(NC(=O)C2(c3ccc4c(c3)OCO4)CC2)cccc1-c1cccc(S(=O)(=O)N(C)C)c1] | 386 |
| 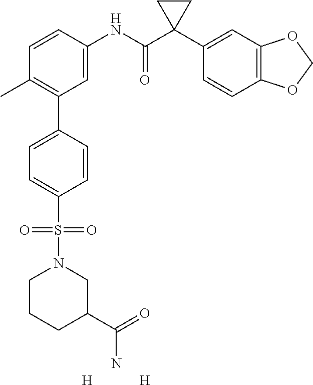 | 387 |
| 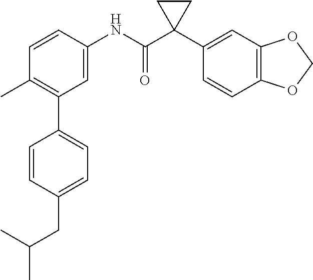 [SMILES: Cc1ccc(NC(=O)C2(c3ccc4c(c3)OCO4)CC2)cc1-c1ccc(CC(C)C)cc1] | 388 |
| 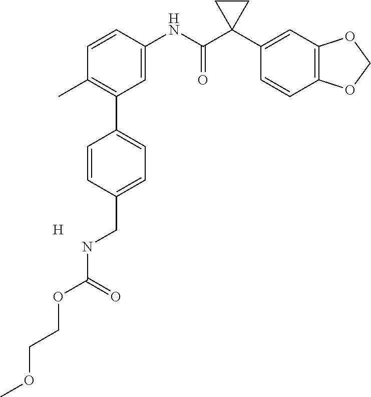 | 389 |
| 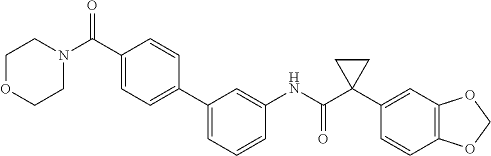 [SMILES: O=C(c1ccc(-c2cccc(NC(=O)C3(c4ccc5c(c4)OCO5)CC3)c2)cc1)N1CCOCC1] | 390 |
| 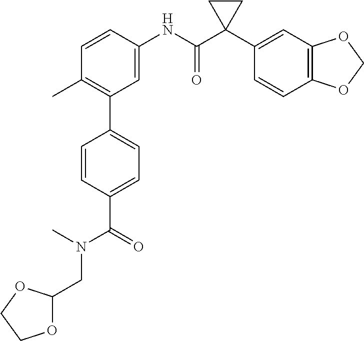 [SMILES: Cc1ccc(NC(=O)C2(c3ccc4c(c3)OCO4)CC2)cc1-c1ccc(C(=O)N(C)CC2OCCO2)cc1] | 391 |
| 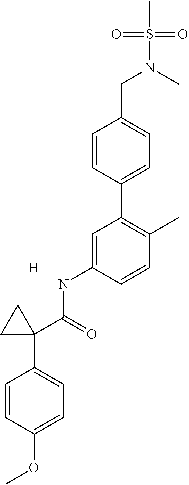 | 392 |
| 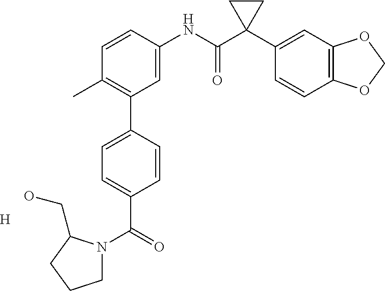 | 393 |
| 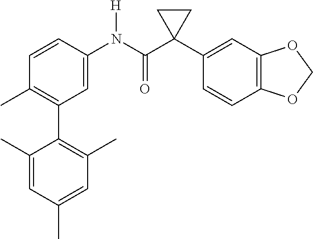 [SMILES: Cc1cc(C)c(-c2cc(NC(=O)C3(c4ccc5c(c4)OCO5)CC3)ccc2C)c(C)c1] | 394 |
| 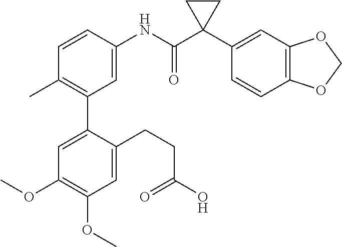 [SMILES: COc1cc(CCC(=O)O)c(-c2cc(NC(=O)C3(c4ccc5c(c4)OCO5)CC3)ccc2C)cc1OC] | 395 |
| 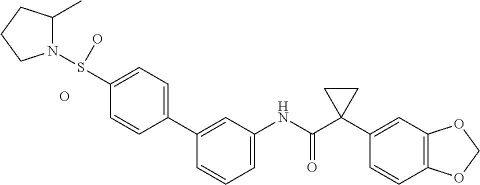 | 396 |
| 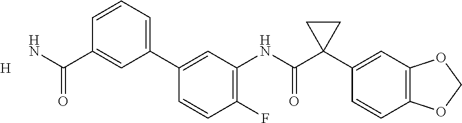 | 397 |
| 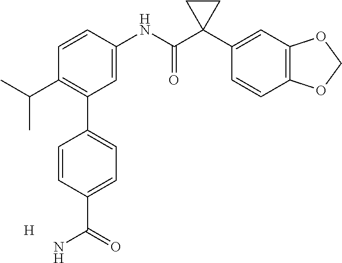 | 398 |
| 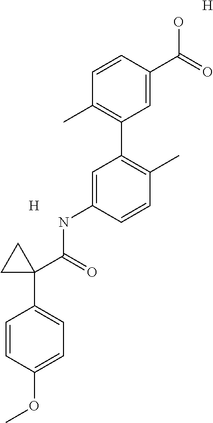 | 399 |
| 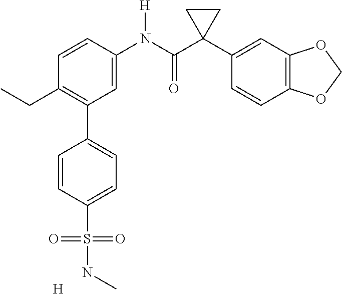 | 400 |
| 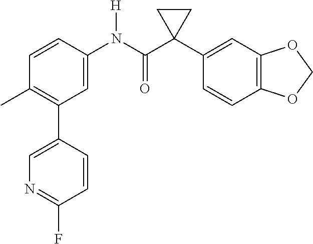 [SMILES: Cc1ccc(NC(=O)C2(c3ccc4c(c3)OCO4)CC2)cc1-c1ccc(F)nc1] | 401 |
| 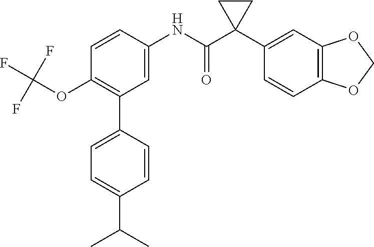 [SMILES: CC(C)c1ccc(-c2cc(NC(=O)C3(c4ccc5c(c4)OCO5)CC3)ccc2OC(F)(F)F)cc1] | 402 |
| 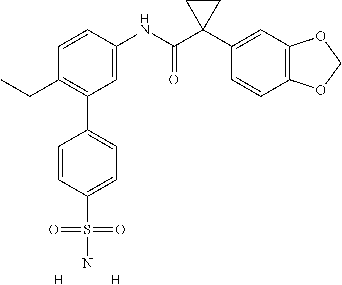 | 403 |
| 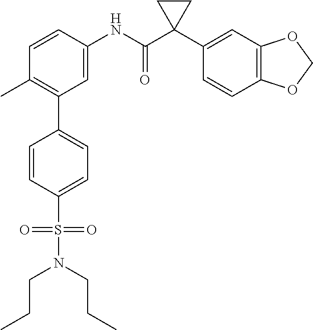 [SMILES: CCCN(CCC)S(=O)(=O)c1ccc(-c2cc(NC(=O)C3(c4ccc5c(c4)OCO5)CC3)ccc2C)cc1] | 404 |
| 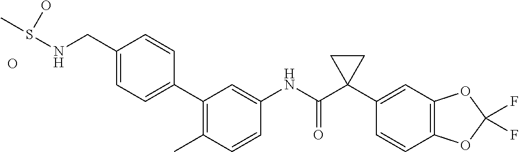 | 405 |
| 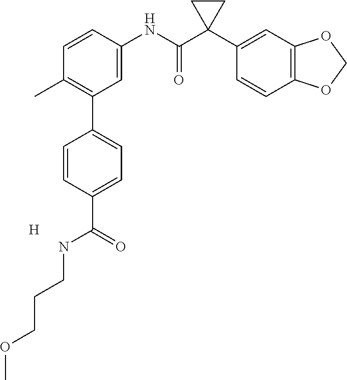 | 406 |
| 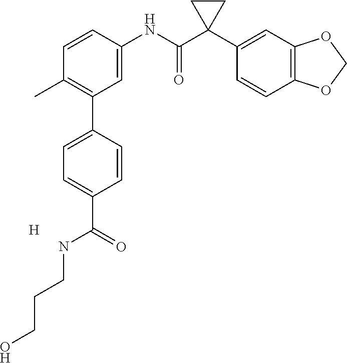 | 407 |
| 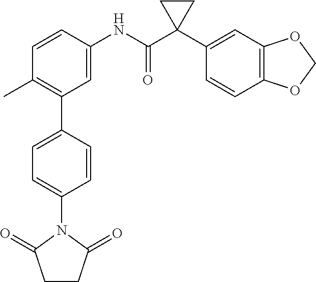 [SMILES: Cc1ccc(NC(=O)C2(c3ccc4c(c3)OCO4)CC2)cc1-c1ccc(N2C(=O)CCC2=O)cc1] | 408 |
| 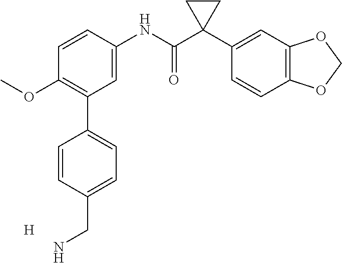 | 409 |
| 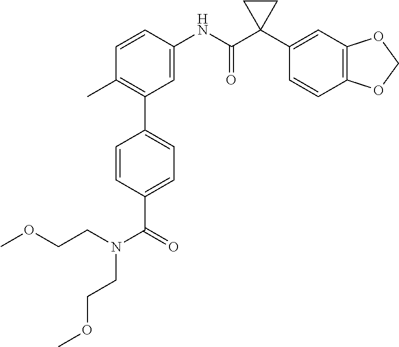 [SMILES: COCCN(CCOC)C(=O)c1ccc(-c2cc(NC(=O)C3(c4ccc5c(c4)OCO5)CC3)ccc2C)cc1] | 410 |
| 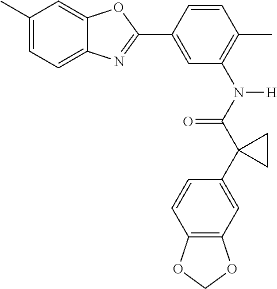 [SMILES: Cc1ccc2nc(-c3ccc(C)c(NC(=O)C4(c5ccc6c(c5)OCO6)CC4)c3)oc2c1] | 411 |
| 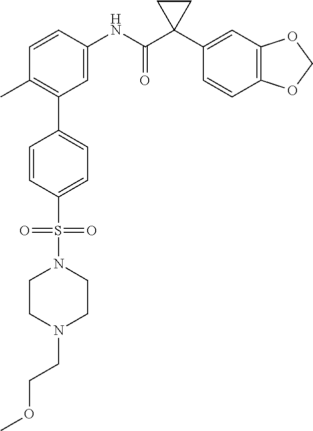 [SMILES: COCCN1CCN(S(=O)(=O)c2ccc(-c3cc(NC(=O)C4(c5ccc6c(c5)OCO6)CC4)ccc3C)cc2)CC1] | 412 |
| 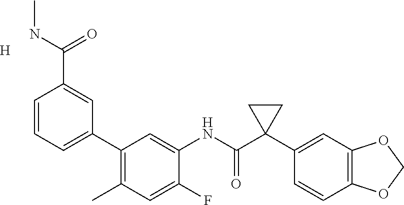 | 413 |
| 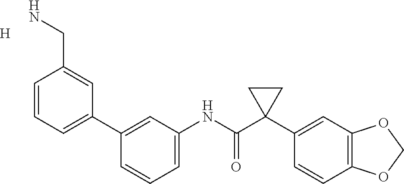 | 414 |
| 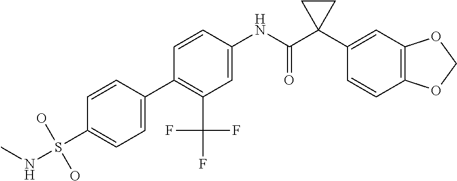 [SMILES: CNS(O)(O)c1ccc(-c2ccc(NC(=O)C3(c4ccc5c(c4)OCO5)CC3)cc2C(F)(F)F)cc1] | 415 |
| 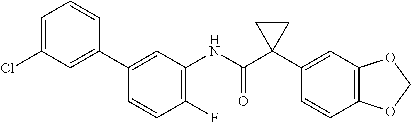 [SMILES: O=C(Nc1cc(-c2cccc(Cl)c2)ccc1F)C1(c2ccc3c(c2)OCO3)CC1] | 416 |
| 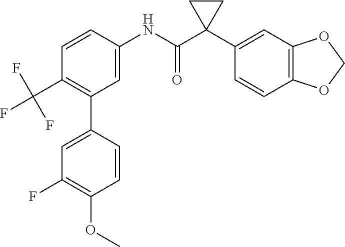 [SMILES: COc1ccc(-c2cc(NC(=O)C3(c4ccc5c(c4)OCO5)CC3)ccc2C(F)(F)F)cc1F] | 417 |
| 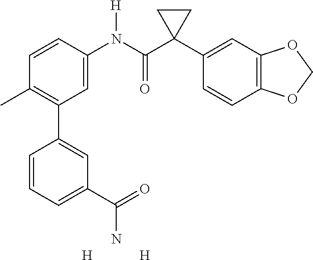 | 418 |
| 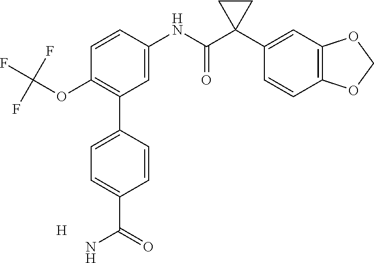 | 419 |
| 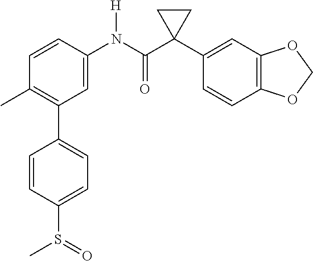 [SMILES: Cc1ccc(NC(=O)C2(c3ccc4c(c3)OCO4)CC2)cc1-c1ccc(S(C)=O)cc1] | 420 |
| 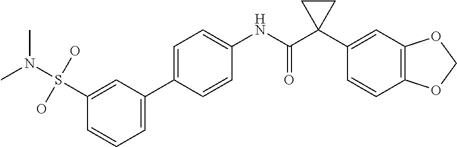 [SMILES: CN(C)S(O)(O)c1cccc(-c2ccc(NC(=O)C3(c4ccc5c(c4)OCO5)CC3)cc2)c1] | 421 |
| 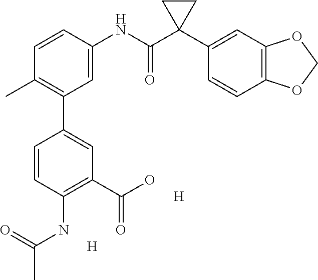 | 422 |
| 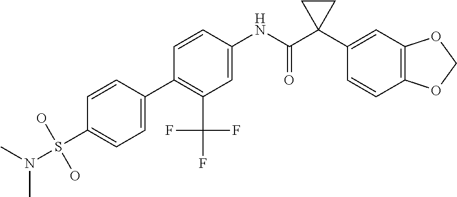 [SMILES: CN(C)S(O)(O)c1ccc(-c2ccc(NC(=O)C3(c4ccc5c(c4)OCO5)CC3)cc2C(F)(F)F)cc1] | 423 |
| 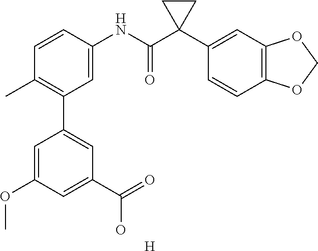 | 424 |
| 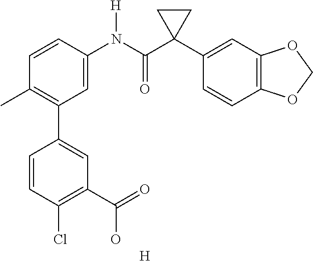 | 425 |
| 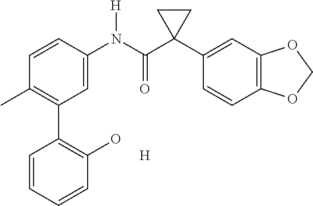 | 426 |
| 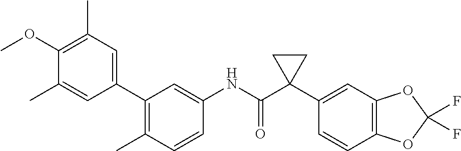 [SMILES: COc1c(C)cc(-c2cc(NC(=O)C3(c4ccc5c(c4)OC(F)(F)O5)CC3)ccc2C)cc1C] | 427 |
| 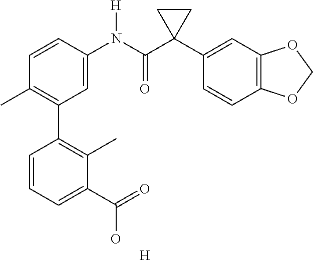 | 428 |
| 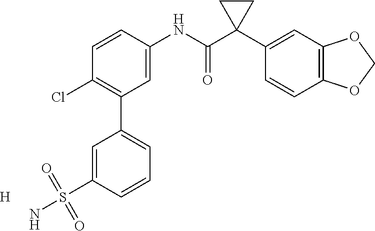 | 429 |
| 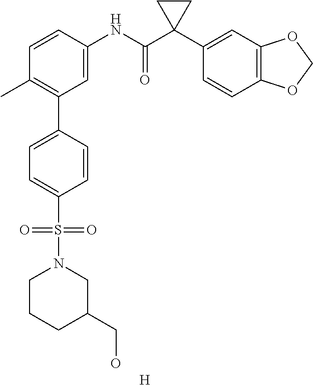 | 430 |
| 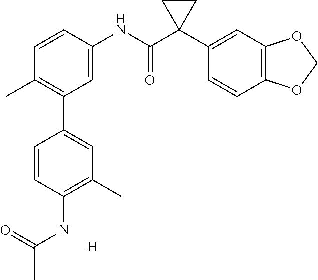 | 431 |
| 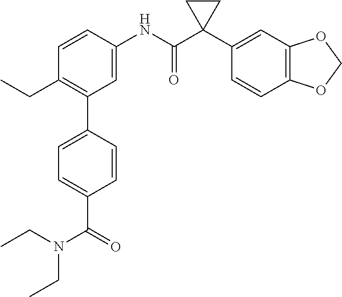 [SMILES: CCc1ccc(NC(=O)C2(c3ccc4c(c3)OCO4)CC2)cc1-c1ccc(C(=O)N(CC)CC)cc1] | 432 |
| 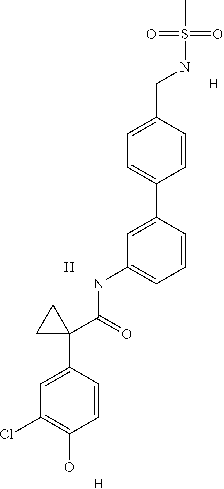 | 433 |
| 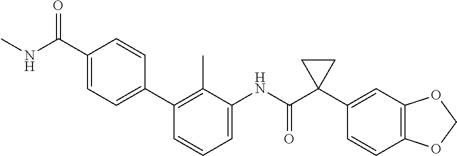 [SMILES: CNC(=O)c1ccc(-c2cccc(NC(=O)C3(c4ccc5c(c4)OCO5)CC3)c2C)cc1] | 434 |
| 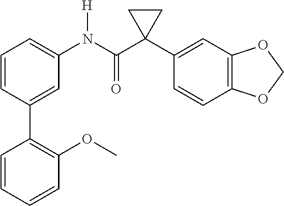 [SMILES: COc1ccccc1-c1cccc(NC(=O)C2(c3ccc4c(c3)OCO4)CC2)c1] | 435 |
| 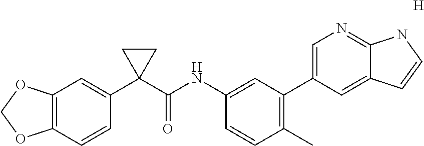 | 436 |
| 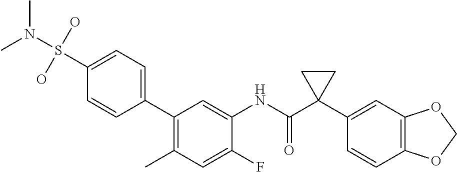 [SMILES: Cc1cc(F)c(NC(=O)C2(c3ccc4c(c3)OCO4)CC2)cc1-c1ccc(S(O)(O)N(C)C)cc1] | 437 |
| 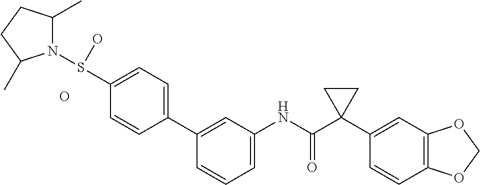 | 438 |
| 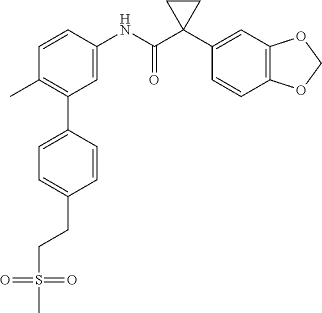 [SMILES: Cc1ccc(NC(=O)C2(c3ccc4c(c3)OCO4)CC2)cc1-c1ccc(CCS(C)(=O)=O)cc1] | 439 |
| 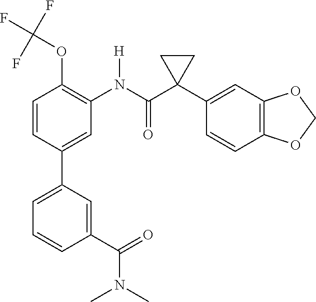 [SMILES: CN(C)C(=O)c1cccc(-c2ccc(OC(F)(F)F)c(NC(=O)C3(c4ccc5c(c4)OCO5)CC3)c2)c1] | 440 |
| 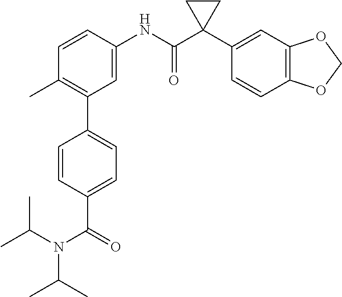 [SMILES: Cc1ccc(NC(=O)C2(c3ccc4c(c3)OCO4)CC2)cc1-c1ccc(C(=O)N(C(C)C)C(C)C)cc1] | 441 |
| 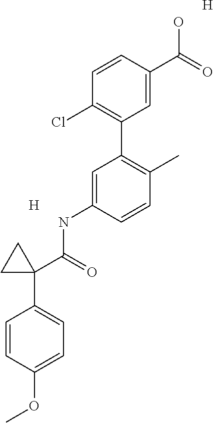 | 442 |
| 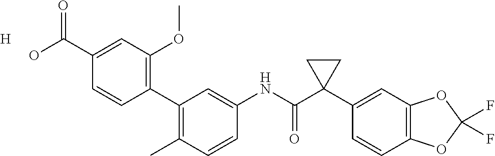 | 443 |
| 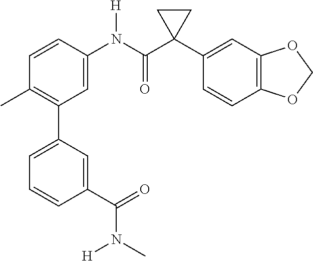 | 444 |
| 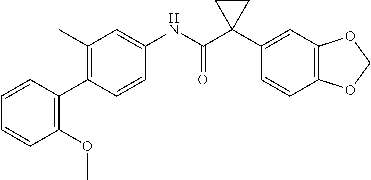 [SMILES: COc1ccccc1-c1ccc(NC(=O)C2(c3ccc4c(c3)OCO4)CC2)cc1C] | 445 |
| 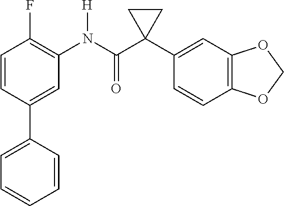 [SMILES: O=C(Nc1cc(-c2ccccc2)ccc1F)C1(c2ccc3c(c2)OCO3)CC1] | 446 |
| 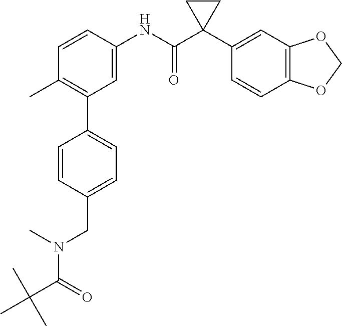 [SMILES: Cc1ccc(NC(=O)C2(c3ccc4c(c3)OCO4)CC2)cc1-c1ccc(CN(C)C(=O)C(C)(C)C)cc1] | 447 |
| 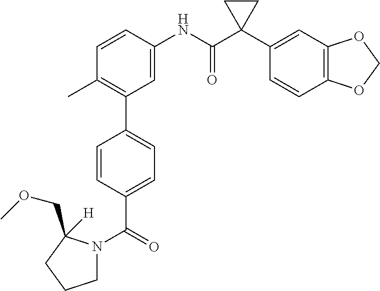 [SMILES: COC[C@@H]1CCCN1C(=O)c1ccc(-c2cc(NC(=O)C3(c4ccc5c(c4)OCO5)CC3)ccc2C)cc1] | 448 |
| 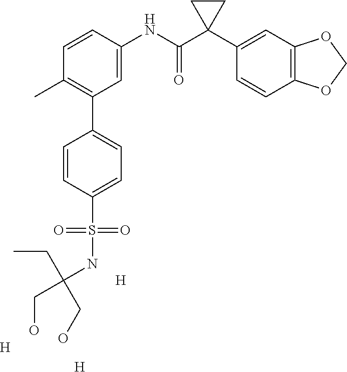 | 449 |
| 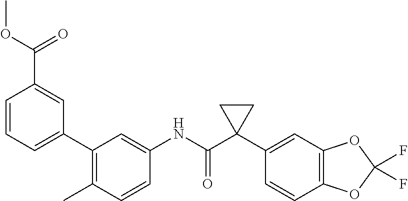 [SMILES: COC(=O)c1cccc(-c2cc(NC(=O)C3(c4ccc5c(c4)OC(F)(F)O5)CC3)ccc2C)c1] | 450 |
| 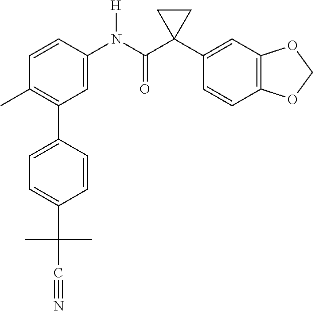 [SMILES: Cc1ccc(NC(=O)C2(c3ccc4c(c3)OCO4)CC2)cc1-c1ccc(C(C)(C)C#N)cc1] | 451 |
| 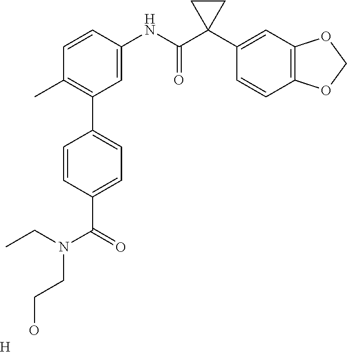 | 452 |
| 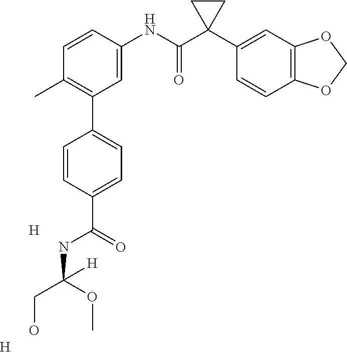 | 453 |
| 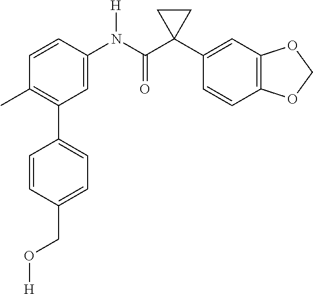 [SMILES: Cc1ccc(NC(=O)C2(c3ccc4c(c3)OCO4)CC2)cc1-c1ccc(CO)cc1] | 454 |
| 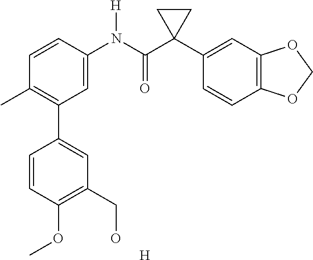 | 455 |
| 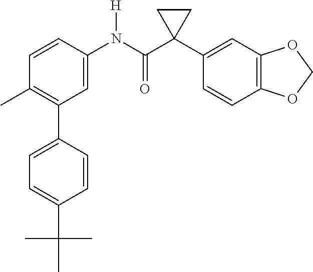 [SMILES: Cc1ccc(NC(=O)C2(c3ccc4c(c3)OCO4)CC2)cc1-c1ccc(C(C)(C)C)cc1] | 456 |
| 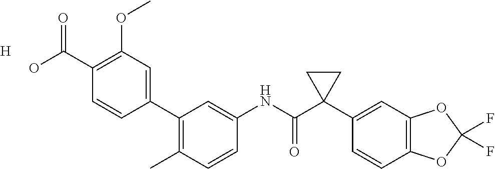 | 457 |
| 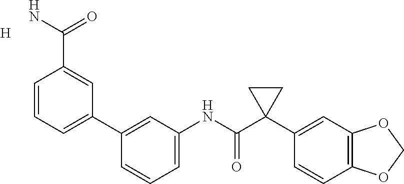 | 458 |
| 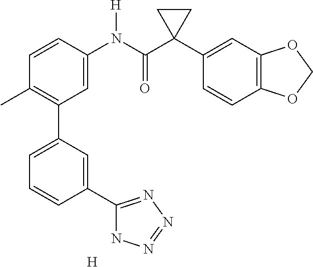 | 459 |
| 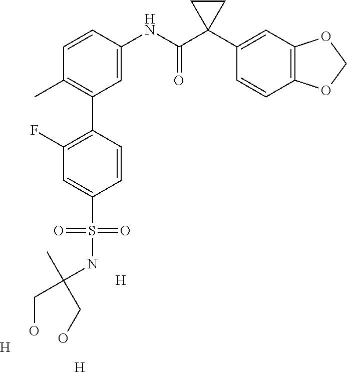 | 460 |
| 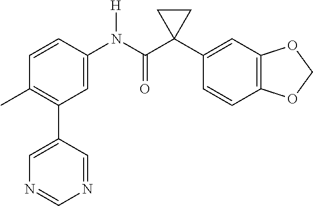 [SMILES: Cc1ccc(NC(=O)C2(c3ccc4c(c3)OCO4)CC2)cc1-c1cncnc1] | 461 |
| 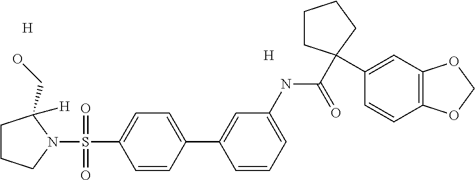 | 462 |
| 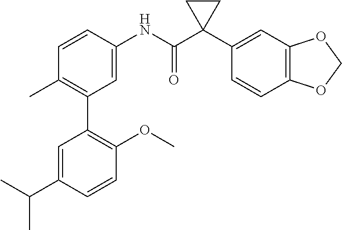 [SMILES: COc1ccc(C(C)C)cc1-c1cc(NC(=O)C2(c3ccc4c(c3)OCO4)CC2)ccc1C] | 463 |
| 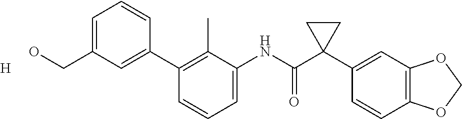 | 464 |
| 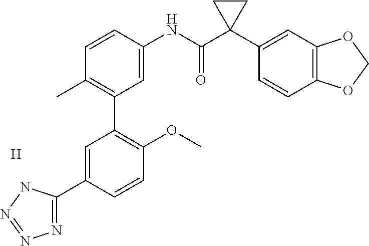 | 465 |
| 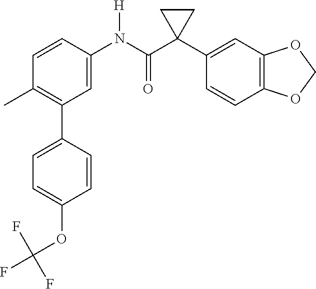 [SMILES: Cc1ccc(NC(=O)C2(c3ccc4c(c3)OCO4)CC2)cc1-c1ccc(OC(F)(F)F)cc1] | 466 |
| 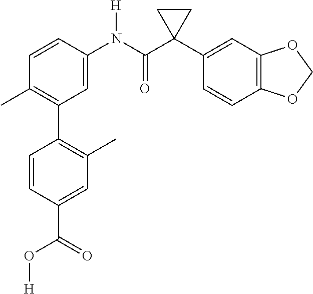 [SMILES: Cc1cc(C(=O)O)ccc1-c1cc(NC(=O)C2(c3ccc4c(c3)OCO4)CC2)ccc1C] | 467 |
| 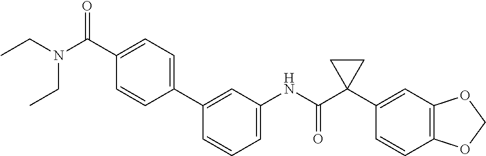 [SMILES: CCN(CC)C(=O)c1ccc(-c2cccc(NC(=O)C3(c4ccc5c(c4)OCO5)CC3)c2)cc1] | 468 |
| 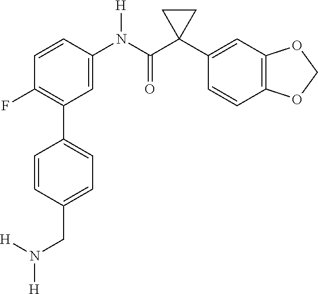 [SMILES: NCc1ccc(-c2cc(NC(=O)C3(c4ccc5c(c4)OCO5)CC3)ccc2F)cc1] | 469 |
| 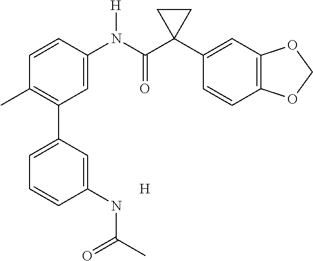 | 470 |
| 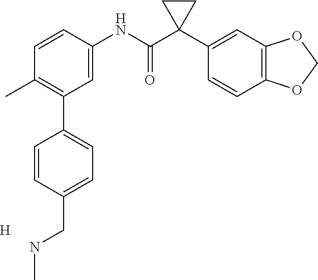 | 471 |
| 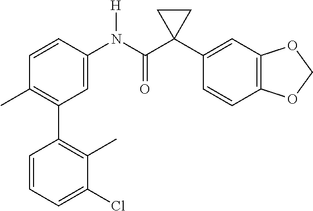 [SMILES: Cc1ccc(NC(=O)C2(c3ccc4c(c3)OCO4)CC2)cc1-c1cccc(Cl)c1C] | 472 |
| 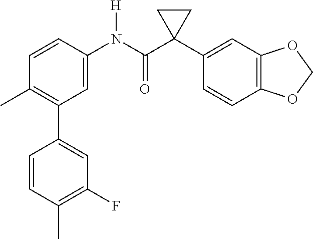 [SMILES: Cc1ccc(-c2cc(NC(=O)C3(c4ccc5c(c4)OCO5)CC3)ccc2C)cc1F] | 473 |
| 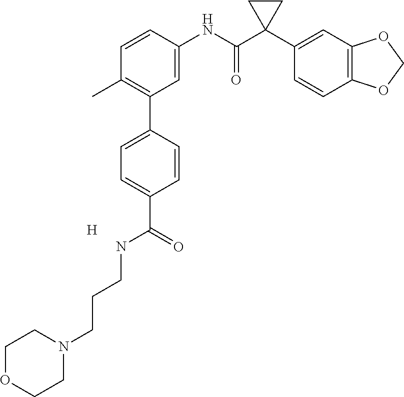 | 474 |
| 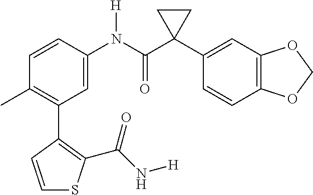 [SMILES: Cc1ccc(NC(=O)C2(c3ccc4c(c3)OCO4)CC2)cc1-c1ccsc1C(N)=O] | 475 |
| 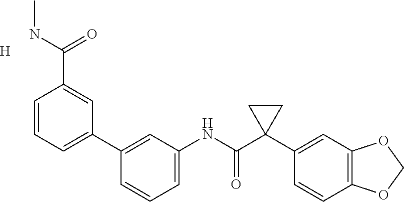 | 476 |
| 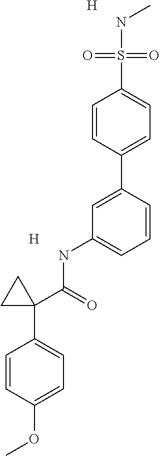 | 477 |
| 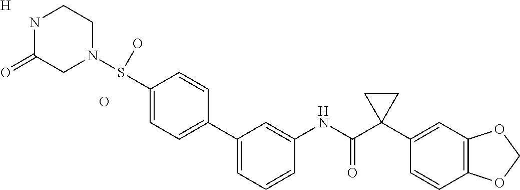 | 478 |
| 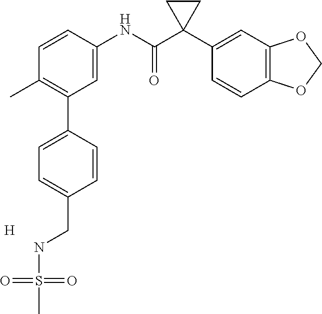 | 479 |
| 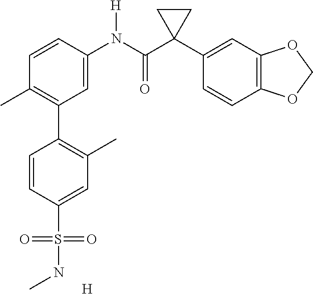 | 480 |
| 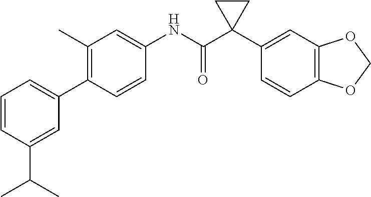 [SMILES: Cc1cc(NC(=O)C2(c3ccc4c(c3)OCO4)CC2)ccc1-c1cccc(C(C)C)c1] | 481 |
| 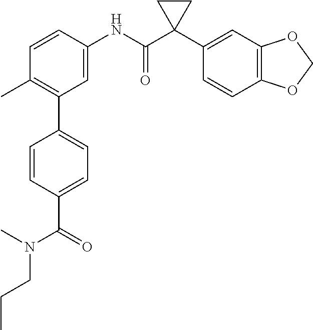 [SMILES: CCCN(C)C(=O)c1ccc(-c2cc(NC(=O)C3(c4ccc5c(c4)OCO5)CC3)ccc2C)cc1] | 482 |
| 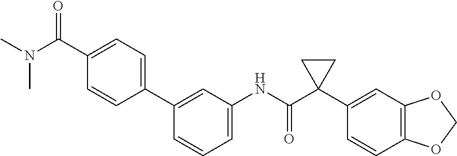 [SMILES: CN(C)C(=O)c1ccc(-c2cccc(NC(=O)C3(c4ccc5c(c4)OCO5)CC3)c2)cc1] | 483 |
| 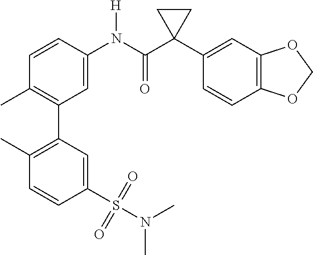 [SMILES: Cc1ccc(NC(=O)C2(c3ccc4c(c3)OCO4)CC2)cc1-c1cc(S(=O)(=O)N(C)C)ccc1C] | 484 |
| 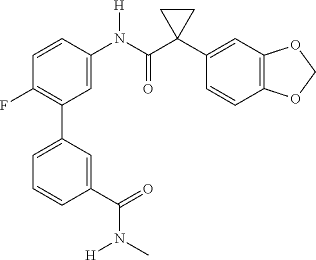 [SMILES: CNC(=O)c1cccc(-c2cc(NC(=O)C3(c4ccc5c(c4)OCO5)CC3)ccc2F)c1] | 485 |
| 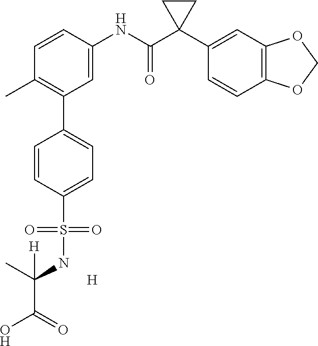 | 486 |
| 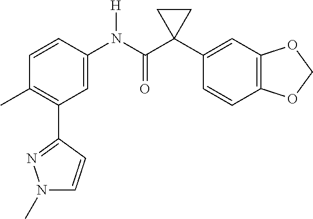 [SMILES: Cc1ccc(NC(=O)C2(c3ccc4c(c3)OCO4)CC2)cc1-c1ccn(C)n1] | 487 |
| 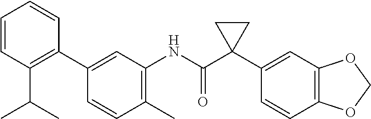 [SMILES: Cc1ccc(-c2ccccc2C(C)C)cc1NC(=O)C1(c2ccc3c(c2)OCO3)CC1] | 488 |
| 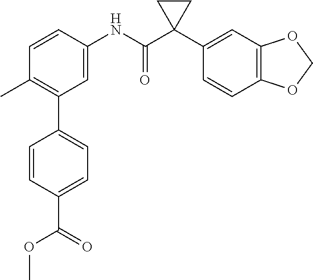 [SMILES: COC(=O)c1ccc(-c2cc(NC(=O)C3(c4ccc5c(c4)OCO5)CC3)ccc2C)cc1] | 489 |
| 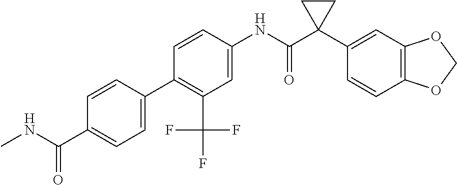 [SMILES: CNC(=O)c1ccc(-c2ccc(NC(=O)C3(c4ccc5c(c4)OCO5)CC3)cc2C(F)(F)F)cc1] | 490 |
| 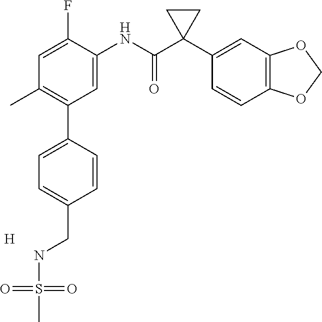 | 491 |
| 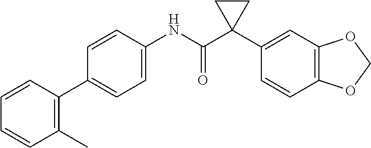 [SMILES: Cc1ccccc1-c1ccc(NC(=O)C2(c3ccc4c(c3)OCO4)CC2)cc1] | 492 |
| 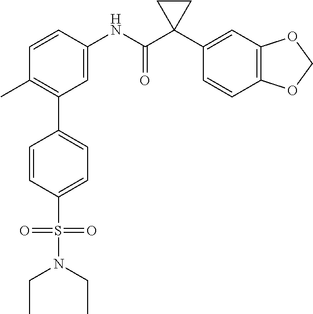 [SMILES: CCN(CC)S(=O)(=O)c1ccc(-c2cc(NC(=O)C3(c4ccc5c(c4)OCO5)CC3)ccc2C)cc1] | 493 |
| 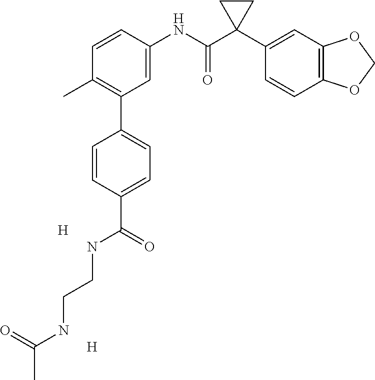 | 494 |
| 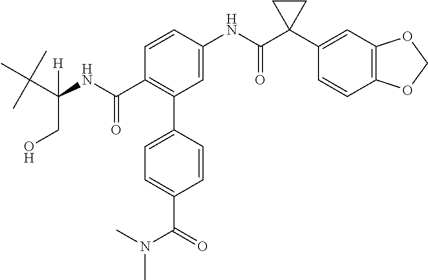 [SMILES: CN(C)C(=O)c1ccc(-c2cc(NC(=O)C3(c4ccc5c(c4)OCO5)CC3)ccc2C(=O)N[C@@H](CO)C(C)(C)C)cc1] | 495 |
| 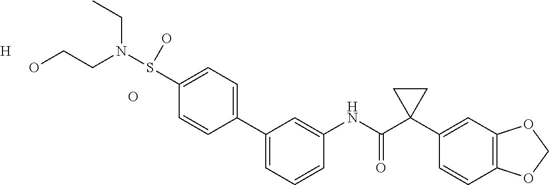 | 496 |
| 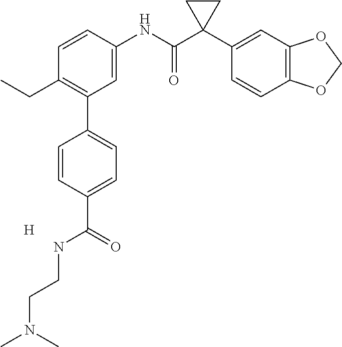 | 497 |
| 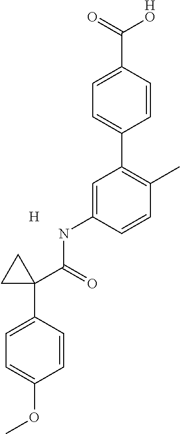 | 498 |
| 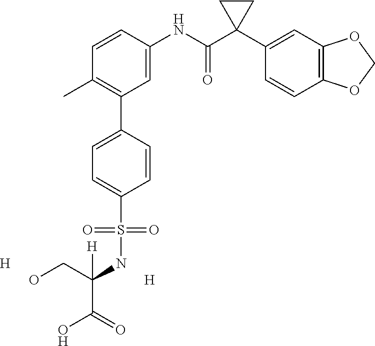 | 499 |
| 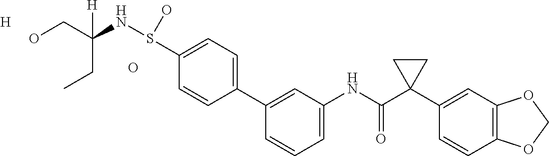 | 500 |
| 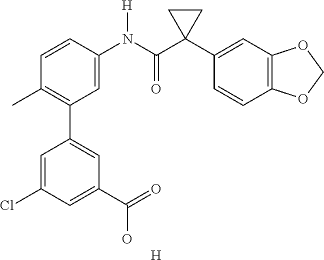 | 501 |
| 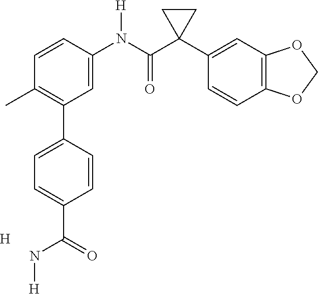 | 502 |
| 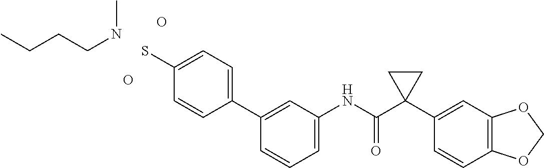 | 503 |
| 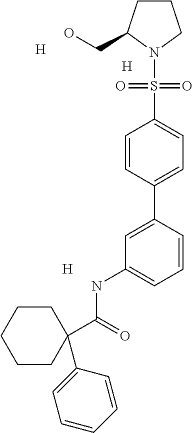 | 504 |
| 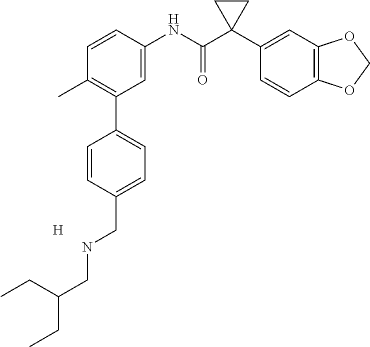 | 505 |
| 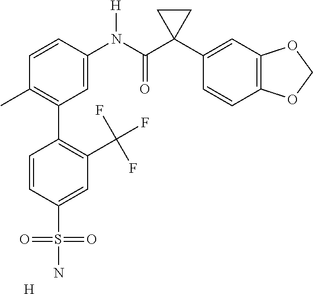 | 506 |
| 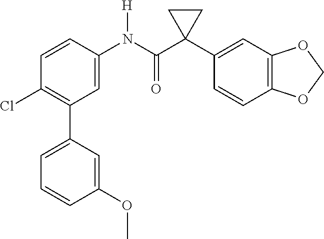 [SMILES: COc1cccc(-c2cc(NC(=O)C3(c4ccc5c(c4)OCO5)CC3)ccc2Cl)c1] | 507 |
| 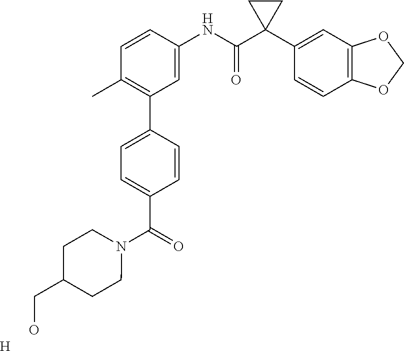 | 508 |
| 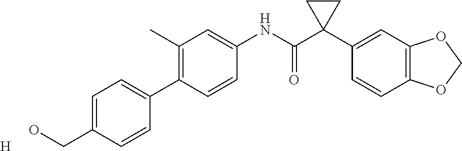 | 509 |
| 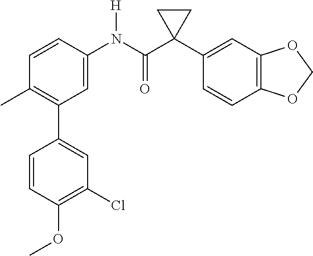 [SMILES: COc1ccc(-c2cc(NC(=O)C3(c4ccc5c(c4)OCO5)CC3)ccc2C)cc1Cl] | 510 |
| 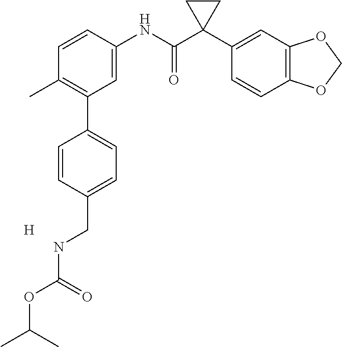 | 511 |
| 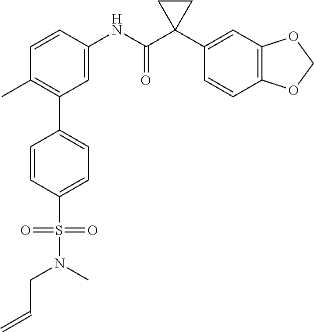 [SMILES: C=CCN(C)S(=O)(=O)c1ccc(-c2cc(NC(=O)C3(c4ccc5c(c4)OCO5)CC3)ccc2C)cc1] | 512 |
| 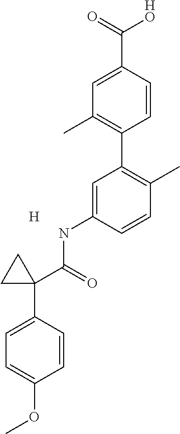 | 513 |
| 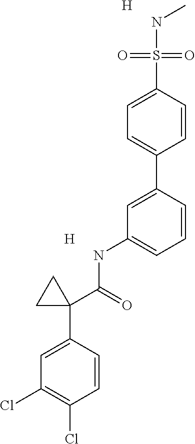 | 514 |
| 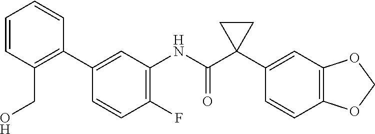 [SMILES: O=C(Nc1cc(-c2ccccc2CO)ccc1F)C1(c2ccc3c(c2)OCO3)CC1] | 515 |
| 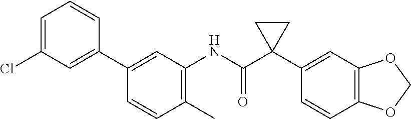 [SMILES: Cc1ccc(-c2cccc(Cl)c2)cc1NC(=O)C1(c2ccc3c(c2)OCO3)CC1] | 516 |
| 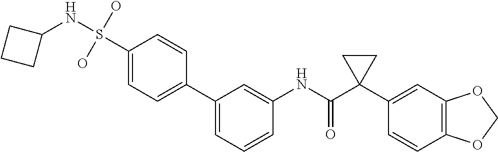 [SMILES: O=C(Nc1cccc(-c2ccc(S(O)(O)NC3CCC3)cc2)c1)C1(c2ccc3c(c2)OCO3)CC1] | 517 |
| 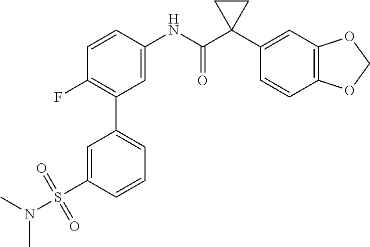 [SMILES: CN(C)S(=O)(=O)c1cccc(-c2cc(NC(=O)C3(c4ccc5c(c4)OCO5)CC3)ccc2F)c1] | 518 |
| 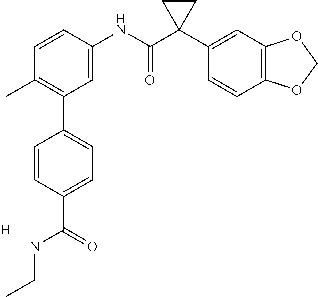 | 519 |
| 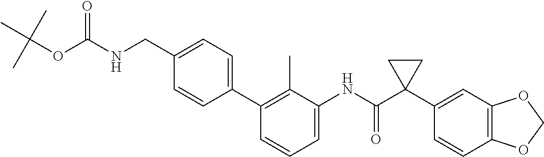 [SMILES: Cc1c(NC(=O)C2(c3ccc4c(c3)OCO4)CC2)cccc1-c1ccc(CNC(=O)OC(C)(C)C)cc1] | 520 |
| 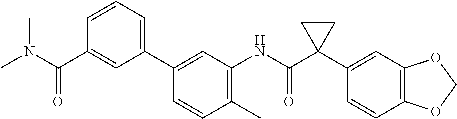 [SMILES: Cc1ccc(-c2cccc(C(=O)N(C)C)c2)cc1NC(=O)C1(c2ccc3c(c2)OCO3)CC1] | 521 |
| 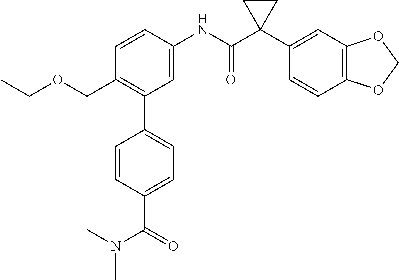 [SMILES: CCOCc1ccc(NC(=O)C2(c3ccc4c(c3)OCO4)CC2)cc1-c1ccc(C(=O)N(C)C)cc1] | 522 |
| 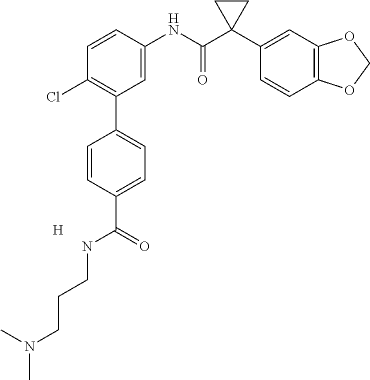 | 523 |
| 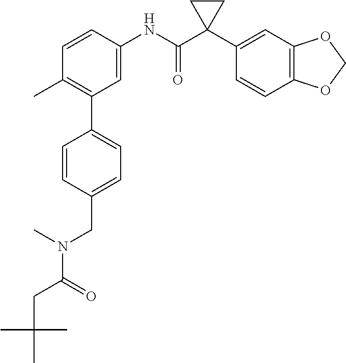 [SMILES: Cc1ccc(NC(=O)C2(c3ccc4c(c3)OCO4)CC2)cc1-c1ccc(CN(C)C(=O)CC(C)(C)C)cc1] | 524 |
| 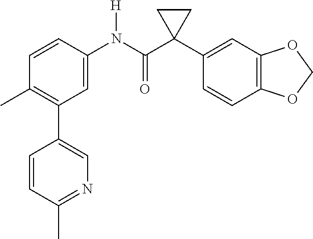 [SMILES: Cc1ccc(-c2cc(NC(=O)C3(c4ccc5c(c4)OCO5)CC3)ccc2C)cn1] | 525 |
| 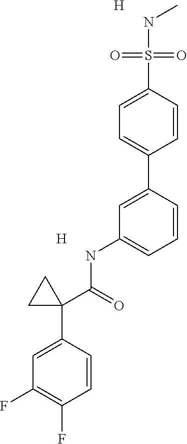 | 526 |
| 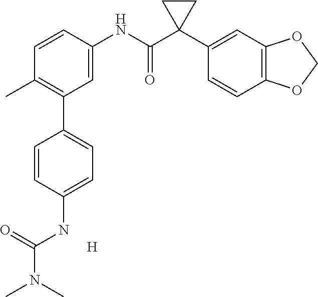 | 527 |
| 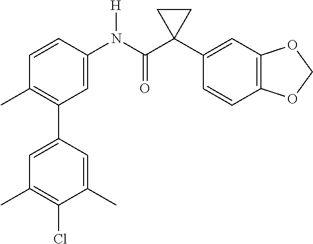 [SMILES: Cc1ccc(NC(=O)C2(c3ccc4c(c3)OCO4)CC2)cc1-c1cc(C)c(Cl)c(C)c1] | 528 |
| 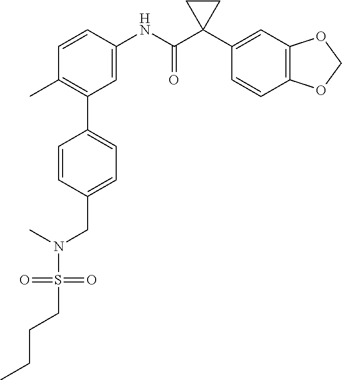 [SMILES: CCCCS(=O)(=O)N(C)Cc1ccc(-c2cc(NC(=O)C3(c4ccc5c(c4)OCO5)CC3)ccc2C)cc1] | 529 |
| 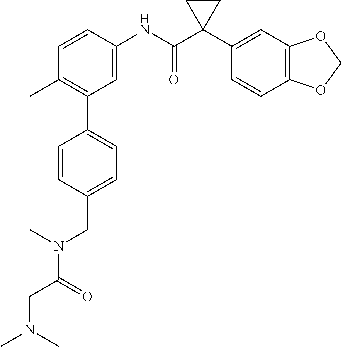 [SMILES: Cc1ccc(NC(=O)C2(c3ccc4c(c3)OCO4)CC2)cc1-c1ccc(CN(C)C(=O)CN(C)C)cc1] | 530 |
| 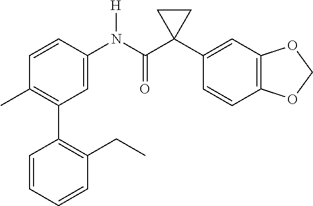 [SMILES: CCc1ccccc1-c1cc(NC(=O)C2(c3ccc4c(c3)OCO4)CC2)ccc1C] | 531 |
| 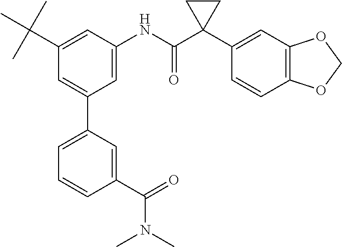 [SMILES: CN(C)C(=O)c1cccc(-c2cc(NC(=O)C3(c4ccc5c(c4)OCO5)CC3)cc(C(C)(C)C)c2)c1] | 532 |
| 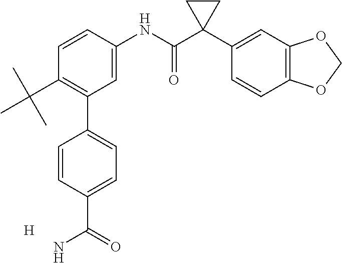 | 533 |
| 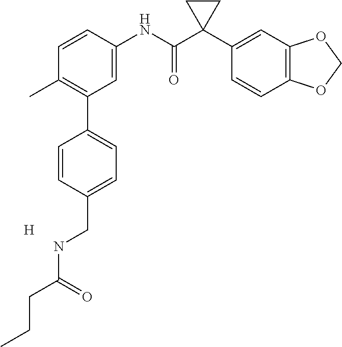 | 534 |
| 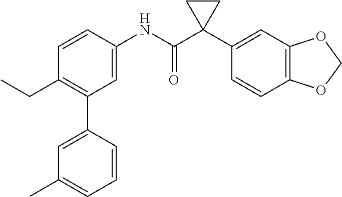 [SMILES: CCc1ccc(NC(=O)C2(c3ccc4c(c3)OCO4)CC2)cc1-c1cccc(C)c1] | 535 |
| 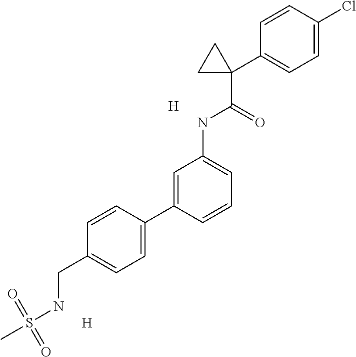 | 536 |
| 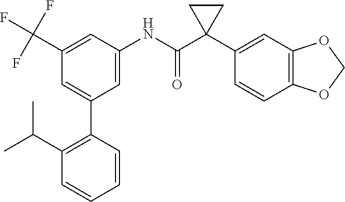 [SMILES: CC(C)c1ccccc1-c1cc(NC(=O)C2(c3ccc4c(c3)OCO4)CC2)cc(C(F)(F)F)c1] | 537 |
| 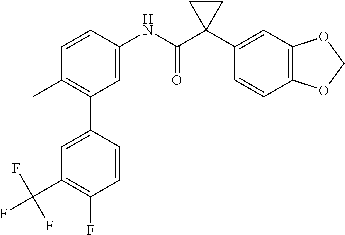 [SMILES: Cc1ccc(NC(=O)C2(c3ccc4c(c3)OCO4)CC2)cc1-c1ccc(F)c(C(F)(F)F)c1] | 538 |
| 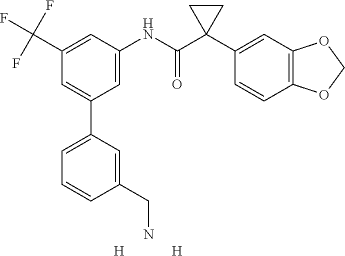 | 539 |
| 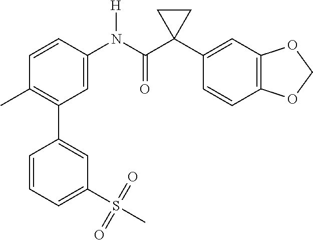 [SMILES: Cc1ccc(NC(=O)C2(c3ccc4c(c3)OCO4)CC2)cc1-c1cccc(S(C)(=O)=O)c1] | 540 |
| 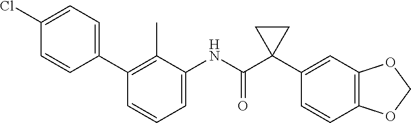 [SMILES: Cc1c(NC(=O)C2(c3ccc4c(c3)OCO4)CC2)cccc1-c1ccc(Cl)cc1] | 541 |
| 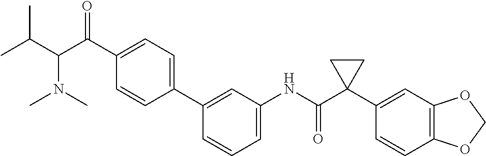 [SMILES: CC(C)C(C(=O)c1ccc(-c2cccc(NC(=O)C3(c4ccc5c(c4)OCO5)CC3)c2)cc1)N(C)C] | 542 |
| 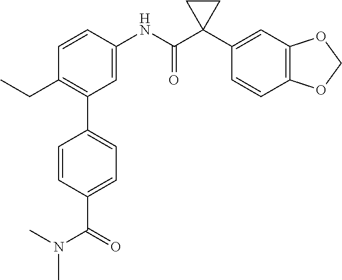 [SMILES: CCc1ccc(NC(=O)C2(c3ccc4c(c3)OCO4)CC2)cc1-c1ccc(C(=O)N(C)C)cc1] | 543 |
| 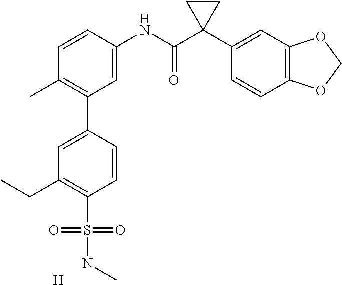 | 544 |
| 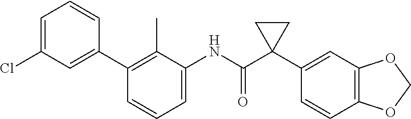 [SMILES: Cc1c(NC(=O)C2(c3ccc4c(c3)OCO4)CC2)cccc1-c1cccc(Cl)c1] | 545 |
| 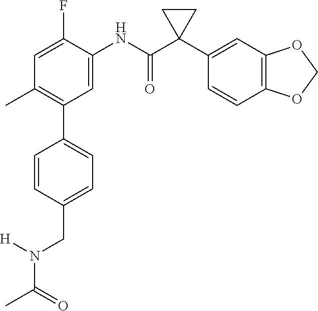 [SMILES: CC(=O)NCc1ccc(-c2cc(NC(=O)C3(c4ccc5c(c4)OCO5)CC3)c(F)cc2C)cc1] | 546 |
| 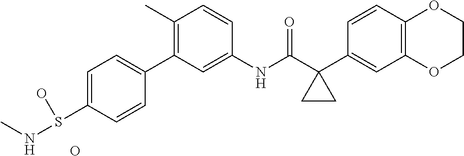 | 547 |
| 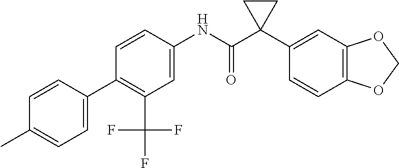 [SMILES: Cc1ccc(-c2ccc(NC(=O)C3(c4ccc5c(c4)OCO5)CC3)cc2C(F)(F)F)cc1] | 548 |
| 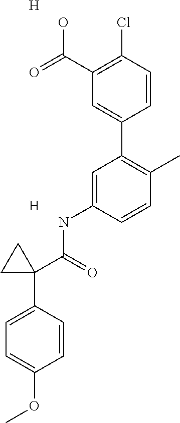 | 549 |
| 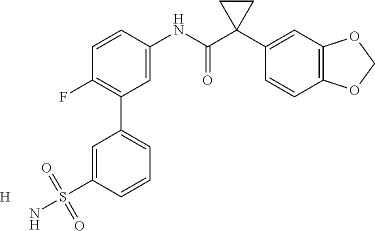 | 550 |
| 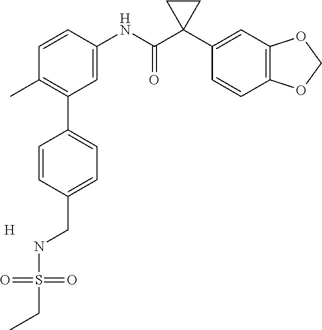 | 551 |
| 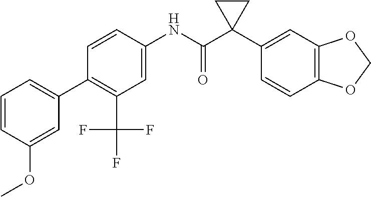 [SMILES: COc1cccc(-c2ccc(NC(=O)C3(c4ccc5c(c4)OCO5)CC3)cc2C(F)(F)F)c1] | 552 |
| 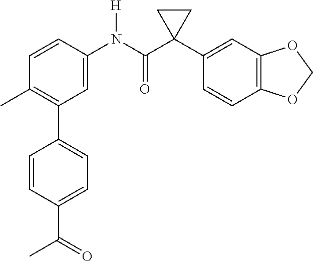 [SMILES: CC(=O)c1ccc(-c2cc(NC(=O)C3(c4ccc5c(c4)OCO5)CC3)ccc2C)cc1] | 553 |
| 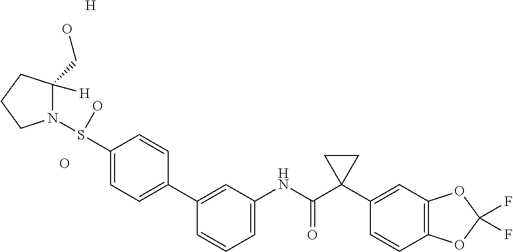 | 554 |
| 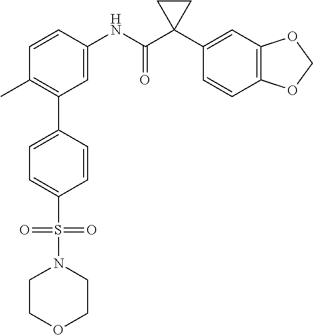 [SMILES: Cc1ccc(NC(=O)C2(c3ccc4c(c3)OCO4)CC2)cc1-c1ccc(S(=O)(=O)N2CCOCC2)cc1] | 555 |
| 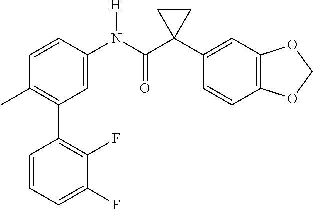 [SMILES: Cc1ccc(NC(=O)C2(c3ccc4c(c3)OCO4)CC2)cc1-c1cccc(F)c1F] | 556 |
| 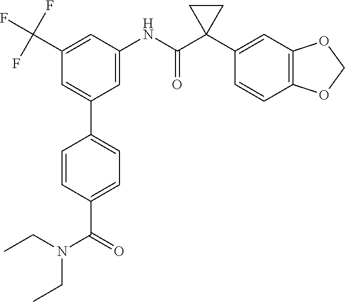 [SMILES: CCN(CC)C(=O)c1ccc(-c2cc(NC(=O)C3(c4ccc5c(c4)OCO5)CC3)cc(C(F)(F)F)c2)cc1] | 557 |
| 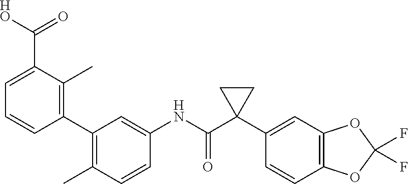 [SMILES: Cc1ccc(NC(=O)C2(c3ccc4c(c3)OC(F)(F)O4)CC2)cc1-c1cccc(C(=O)O)c1C] | 558 |
| 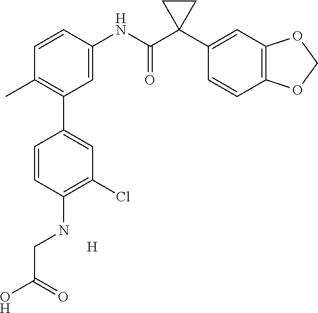 | 559 |
| 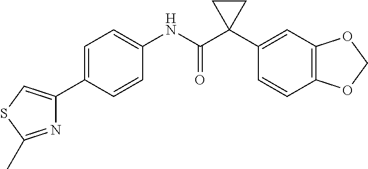 [SMILES: Cc1nc(-c2ccc(NC(=O)C3(c4ccc5c(c4)OCO5)CC3)cc2)cs1] | 560 |
| 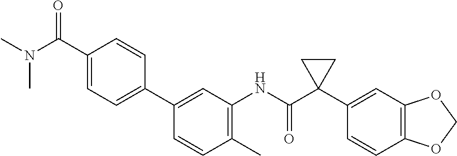 [SMILES: Cc1ccc(-c2ccc(C(=O)N(C)C)cc2)cc1NC(=O)C1(c2ccc3c(c2)OCO3)CC1] | 561 |
| 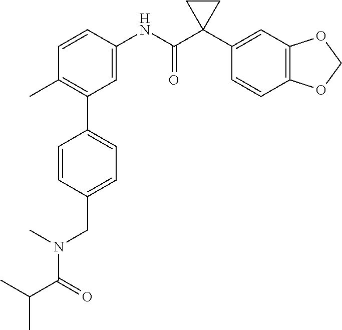 [SMILES: Cc1ccc(NC(=O)C2(c3ccc4c(c3)OCO4)CC2)cc1-c1ccc(CN(C)C(=O)C(C)C)cc1] | 562 |
| 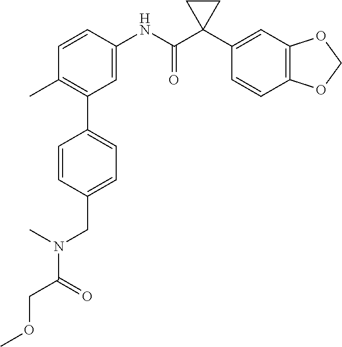 [SMILES: COCC(=O)N(C)Cc1ccc(-c2cc(NC(=O)C3(c4ccc5c(c4)OCO5)CC3)ccc2C)cc1] | 563 |
| 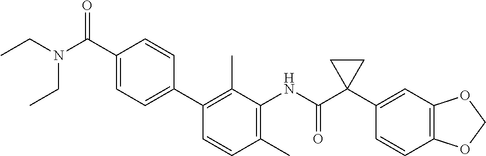 [SMILES: CCN(CC)C(=O)c1ccc(-c2ccc(C)c(NC(=O)C3(c4ccc5c(c4)OCO5)CC3)c2C)cc1] | 564 |
| 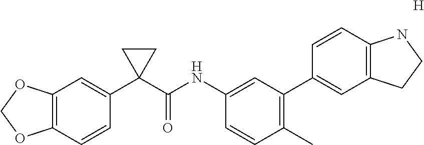 | 565 |
| 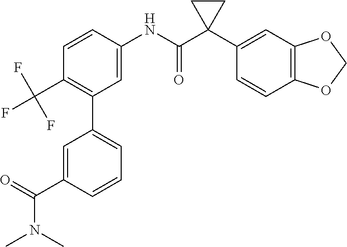 [SMILES: CN(C)C(=O)c1cccc(-c2cc(NC(=O)C3(c4ccc5c(c4)OCO5)CC3)ccc2C(F)(F)F)c1] | 566 |
| 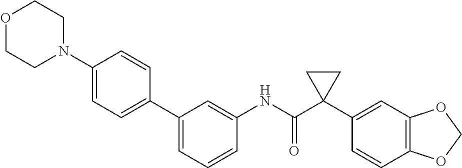 [SMILES: O=C(Nc1cccc(-c2ccc(N3CCOCC3)cc2)c1)C1(c2ccc3c(c2)OCO3)CC1] | 567 |
| 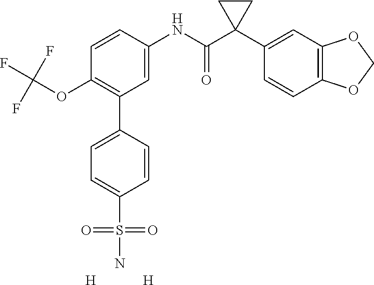 | 568 |
| 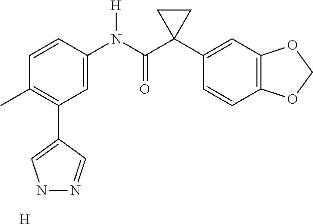 | 569 |
| 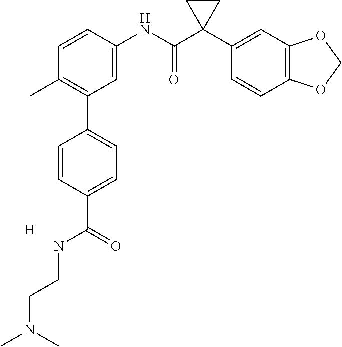 | 570 |
| 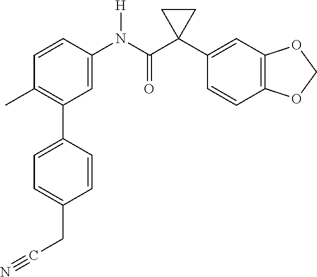 [SMILES: Cc1ccc(NC(=O)C2(c3ccc4c(c3)OCO4)CC2)cc1-c1ccc(CC#N)cc1] | 571 |
| 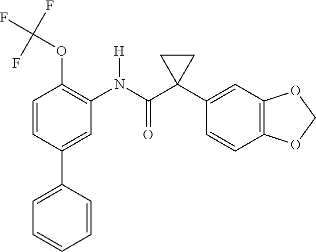 [SMILES: O=C(Nc1cc(-c2ccccc2)ccc1OC(F)(F)F)C1(c2ccc3c(c2)OCO3)CC1] | 572 |
| 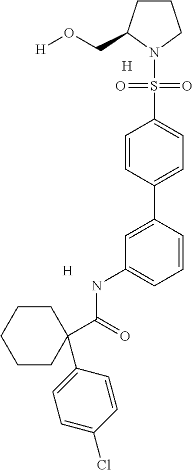 | 573 |
| 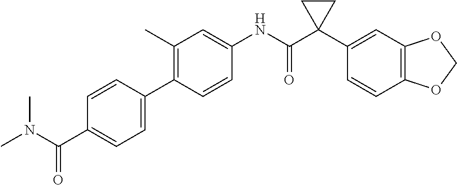 [SMILES: Cc1cc(NC(=O)C2(c3ccc4c(c3)OCO4)CC2)ccc1-c1ccc(C(=O)N(C)C)cc1] | 574 |
| 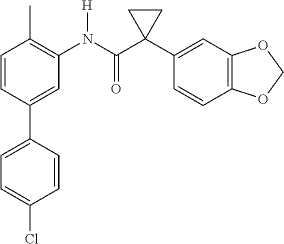 [SMILES: Cc1ccc(-c2ccc(Cl)cc2)cc1NC(=O)C1(c2ccc3c(c2)OCO3)CC1] | 575 |
| 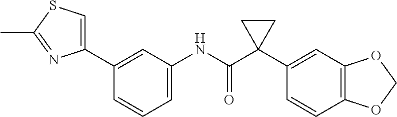 [SMILES: Cc1nc(-c2cccc(NC(=O)C3(c4ccc5c(c4)OCO5)CC3)c2)cs1] | 576 |
| 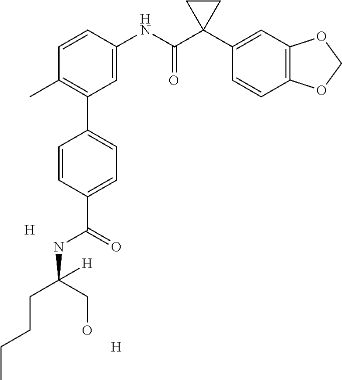 | 577 |
| 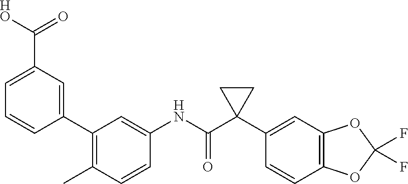 [SMILES: Cc1ccc(NC(=O)C2(c3ccc4c(c3)OC(F)(F)O4)CC2)cc1-c1cccc(C(=O)O)c1] | 578 |
| 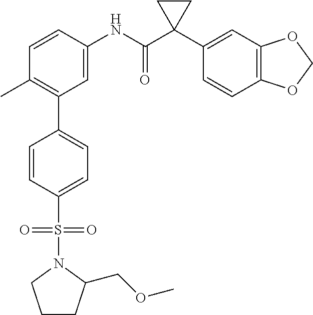 [SMILES: COCC1CCCN1S(=O)(=O)c1ccc(-c2cc(NC(=O)C3(c4ccc5c(c4)OCO5)CC3)ccc2C)cc1] | 579 |
| 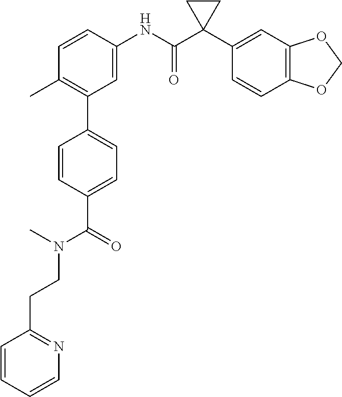 [SMILES: Cc1ccc(NC(=O)C2(c3ccc4c(c3)OCO4)CC2)cc1-c1ccc(C(=O)N(C)CCc2ccccn2)cc1] | 580 |
| 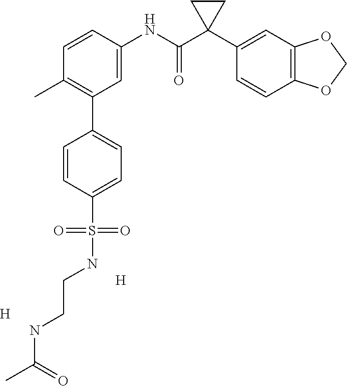 | 581 |
| 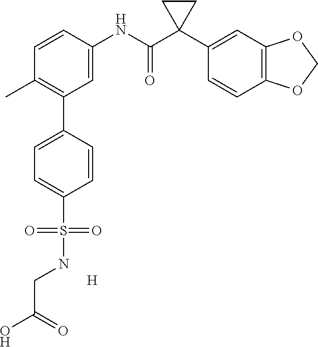 | 582 |
| 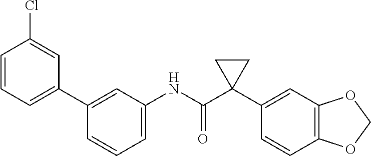 [SMILES: O=C(Nc1cccc(-c2cccc(Cl)c2)c1)C1(c2ccc3c(c2)OCO3)CC1] | 583 |
| 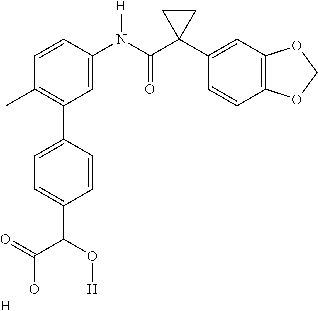 | 584 |
| 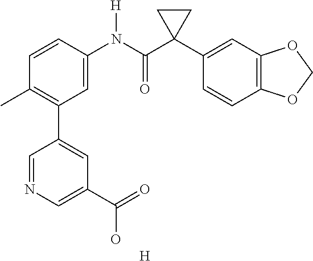 | 585 |
| 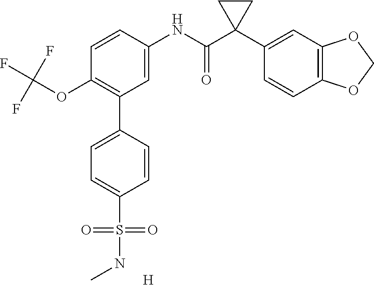 | 586 |
| 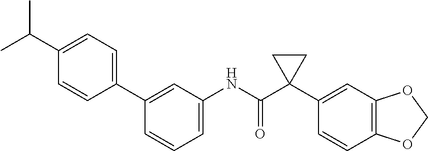 [SMILES: CC(C)c1ccc(-c2cccc(NC(=O)C3(c4ccc5c(c4)OCO5)CC3)c2)cc1] | 587 |
| 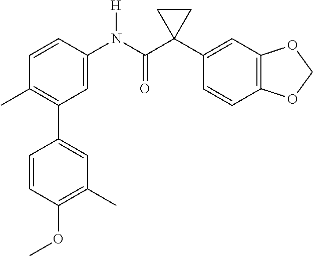 [SMILES: COc1ccc(-c2cc(NC(=O)C3(c4ccc5c(c4)OCO5)CC3)ccc2C)cc1C] | 588 |
| 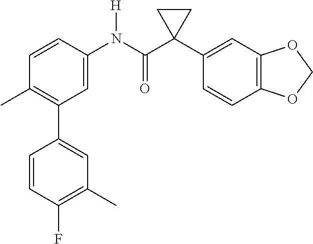 [SMILES: Cc1cc(-c2cc(NC(=O)C3(c4ccc5c(c4)OCO5)CC3)ccc2C)ccc1F] | 589 |
| 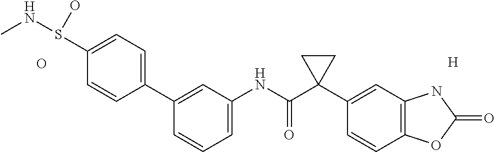 | 590 |
| 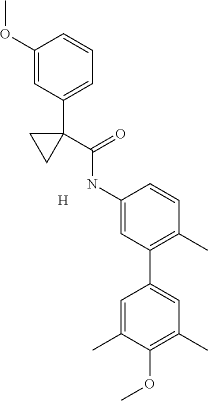 | 591 |
| 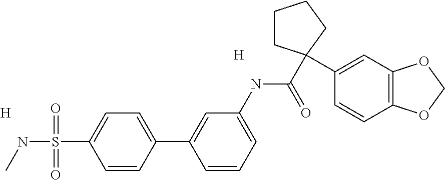 | 592 |
| 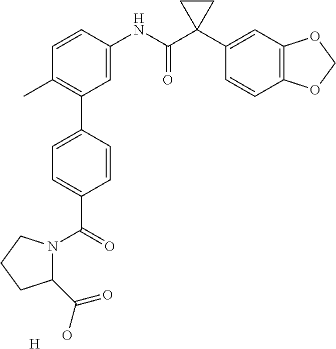 | 593 |
| 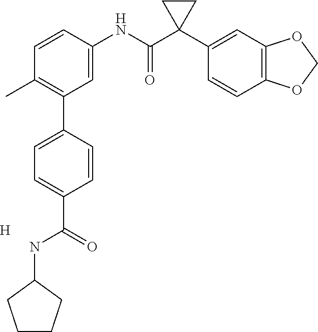 | 594 |
| 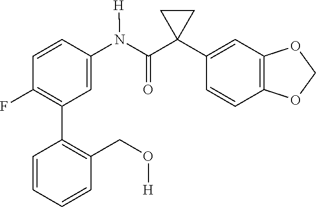 [SMILES: O=C(Nc1ccc(F)c(-c2ccccc2CO)c1)C1(c2ccc3c(c2)OCO3)CC1] | 595 |
| 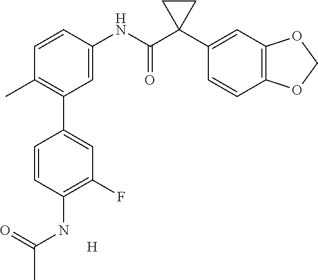 | 596 |
| 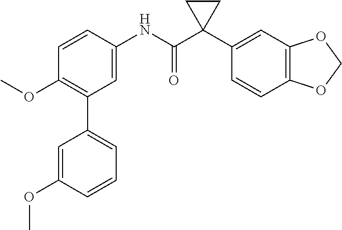 [SMILES: COc1cccc(-c2cc(NC(=O)C3(c4ccc5c(c4)OCO5)CC3)ccc2OC)c1] | 597 |
| 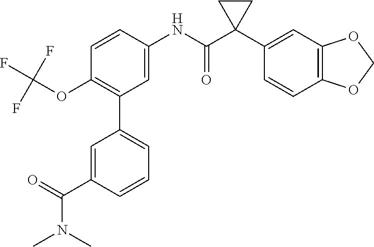 [SMILES: CN(C)C(=O)c1cccc(-c2cc(NC(=O)C3(c4ccc5c(c4)OCO5)CC3)ccc2OC(F)(F)F)c1] | 598 |
| 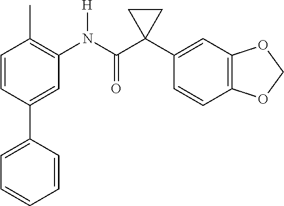 [SMILES: Cc1ccc(-c2ccccc2)cc1NC(=O)C1(c2ccc3c(c2)OCO3)CC1] | 599 |
| 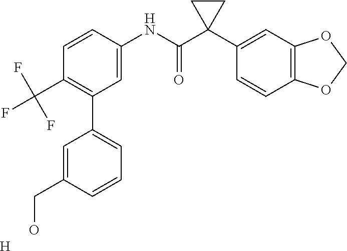 | 600 |
| 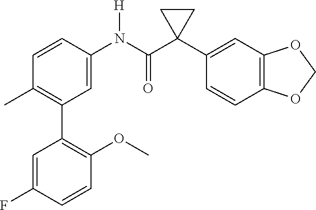 [SMILES: COc1ccc(F)cc1-c1cc(NC(=O)C2(c3ccc4c(c3)OCO4)CC2)ccc1C] | 601 |
| 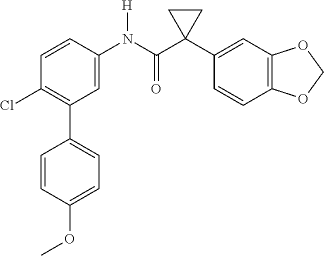 [SMILES: COc1ccc(-c2cc(NC(=O)C3(c4ccc5c(c4)OCO5)CC3)ccc2Cl)cc1] | 602 |
| 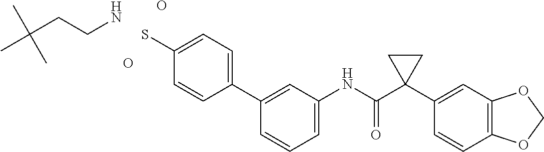 | 603 |
| 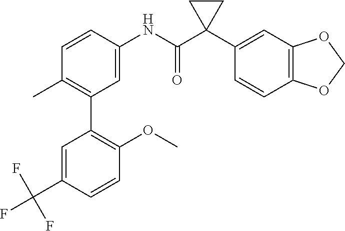 [SMILES: COc1ccc(C(F)(F)F)cc1-c1cc(NC(=O)C2(c3ccc4c(c3)OCO4)CC2)ccc1C] | 604 |
| 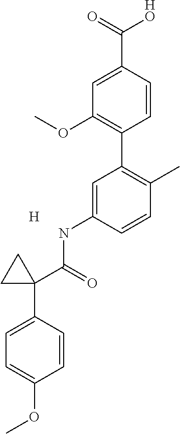 | 605 |
| 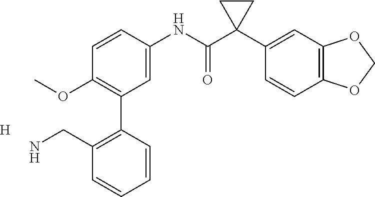 | 606 |
| 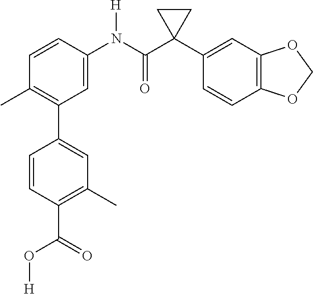 [SMILES: Cc1cc(-c2cc(NC(=O)C3(c4ccc5c(c4)OCO5)CC3)ccc2C)ccc1C(=O)O] | 607 |
| 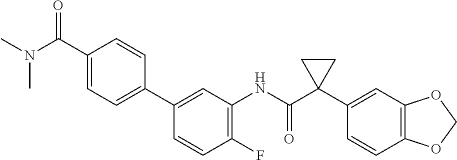 [SMILES: CN(C)C(=O)c1ccc(-c2ccc(F)c(NC(=O)C3(c4ccc5c(c4)OCO5)CC3)c2)cc1] | 608 |
| 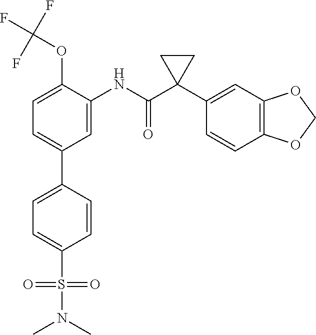 [SMILES: CN(C)S(=O)(=O)c1ccc(-c2ccc(OC(F)(F)F)c(NC(=O)C3(c4ccc5c(c4)OCO5)CC3)c2)cc1] | 609 |
| 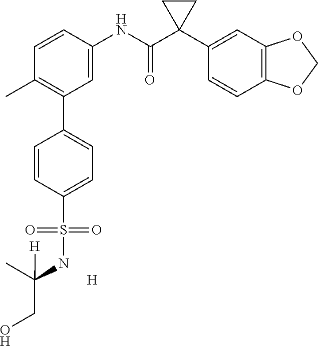 | 610 |
| 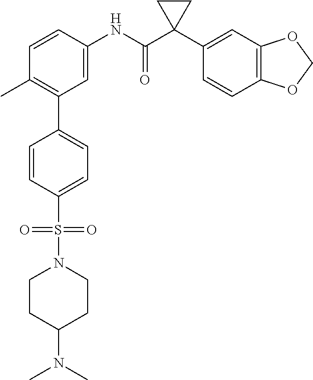 [SMILES: Cc1ccc(NC(=O)C2(c3ccc4c(c3)OCO4)CC2)cc1-c1ccc(S(=O)(=O)N2CCC(N(C)C)CC2)cc1] | 611 |
| 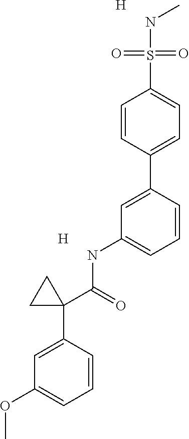 | 612 |
| 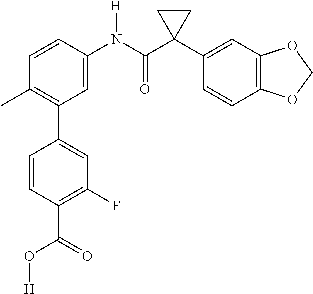 [SMILES: Cc1ccc(NC(=O)C2(c3ccc4c(c3)OCO4)CC2)cc1-c1ccc(C(=O)O)c(F)c1] | 613 |
| 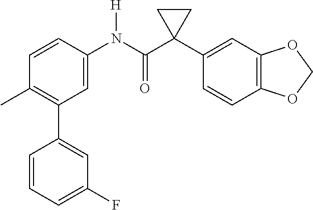 [SMILES: Cc1ccc(NC(=O)C2(c3ccc4c(c3)OCO4)CC2)cc1-c1cccc(F)c1] | 614 |
| 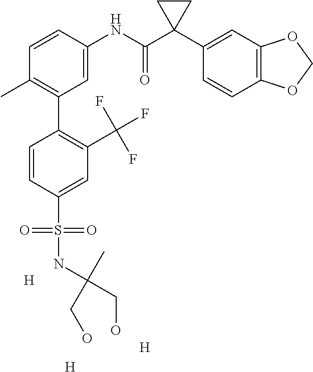 | 615 |
| 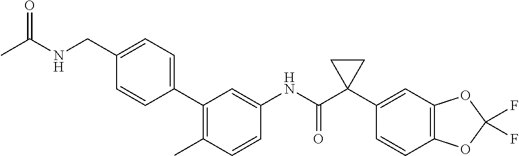 [SMILES: CC(=O)NCc1ccc(-c2cc(NC(=O)C3(c4ccc5c(c4)OC(F)(F)O5)CC3)ccc2C)cc1] | 616 |
| 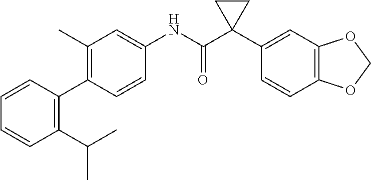 [SMILES: Cc1cc(NC(=O)C2(c3ccc4c(c3)OCO4)CC2)ccc1-c1ccccc1C(C)C] | 617 |
| 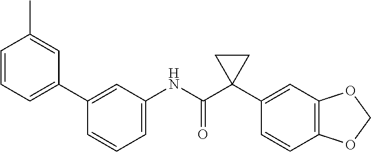 [SMILES: Cc1cccc(-c2cccc(NC(=O)C3(c4ccc5c(c4)OCO5)CC3)c2)c1] | 618 |
| 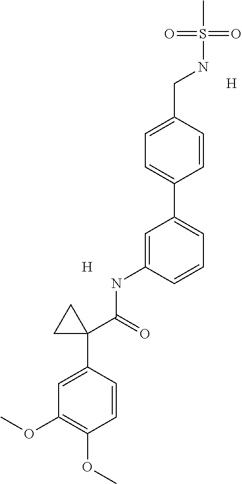 | 619 |
| 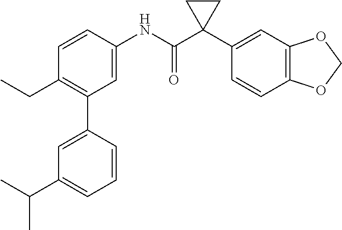 [SMILES: CCc1ccc(NC(=O)C2(c3ccc4c(c3)OCO4)CC2)cc1-c1cccc(C(C)C)c1] | 620 |
| 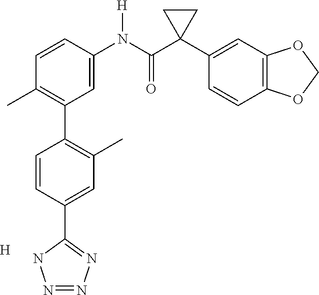 | 621 |
| 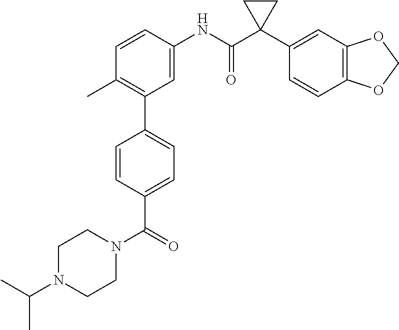 [SMILES: Cc1ccc(NC(=O)C2(c3ccc4c(c3)OCO4)CC2)cc1-c1ccc(C(=O)N2CCN(C(C)C)CC2)cc1] | 622 |
| 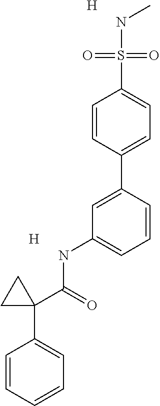 | 623 |
| 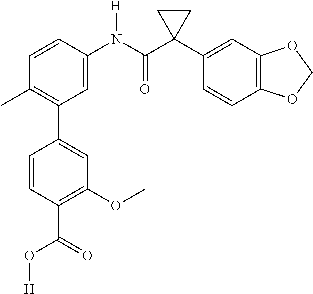 [SMILES: COc1cc(-c2cc(NC(=O)C3(c4ccc5c(c4)OCO5)CC3)ccc2C)ccc1C(=O)O] | 624 |
| 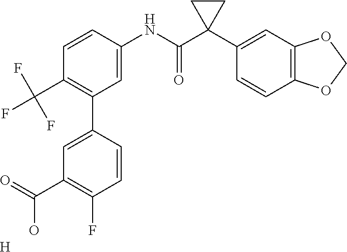 | 625 |
| 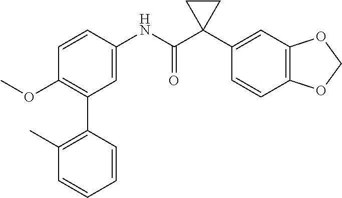 [SMILES: COc1ccc(NC(=O)C2(c3ccc4c(c3)OCO4)CC2)cc1-c1ccccc1C] | 626 |
| 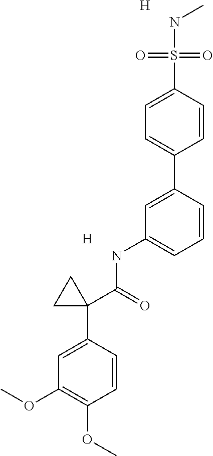 | 627 |
| 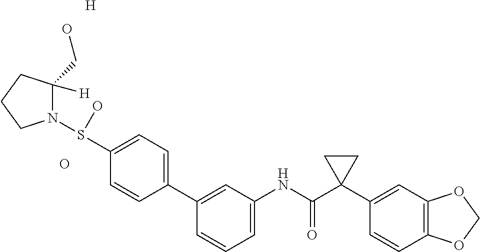 | 628 |
| 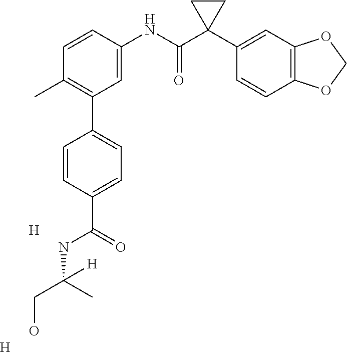 | 629 |
| 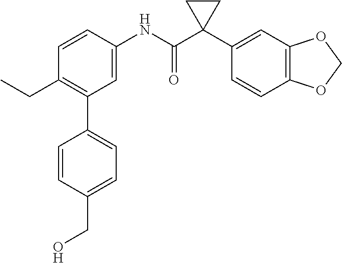 [SMILES: CCc1ccc(NC(=O)C2(c3ccc4c(c3)OCO4)CC2)cc1-c1ccc(CO)cc1] | 630 |
| 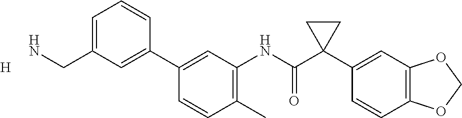 | 631 |
| 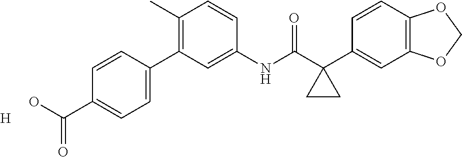 | 632 |
| 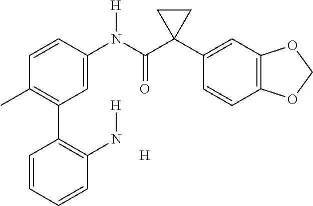 | 633 |
| 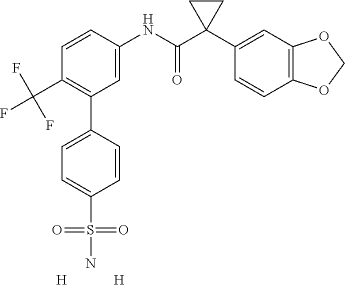 | 634 |
| 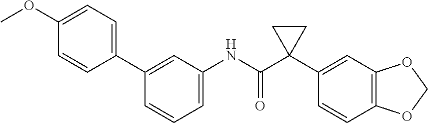 [SMILES: COc1ccc(-c2cccc(NC(=O)C3(c4ccc5c(c4)OCO5)CC3)c2)cc1] | 635 |
| 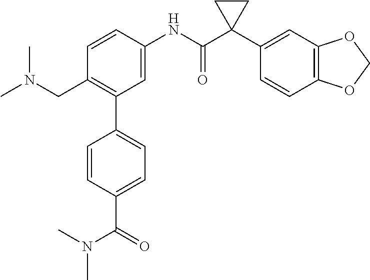 [SMILES: CN(C)Cc1ccc(NC(=O)C2(c3ccc4c(c3)OCO4)CC2)cc1-c1ccc(C(=O)N(C)C)cc1] | 636 |
| 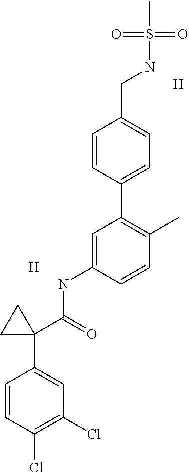 | 637 |
| 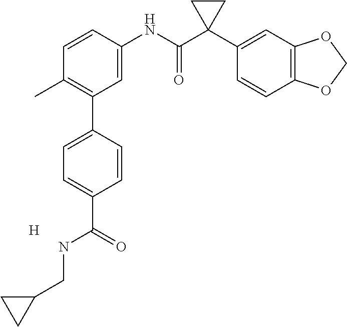 | 638 |
| 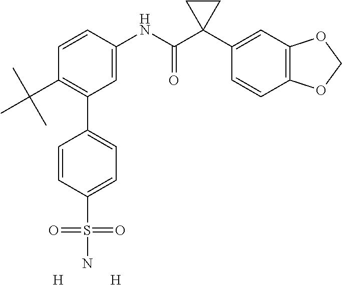 | 639 |
| 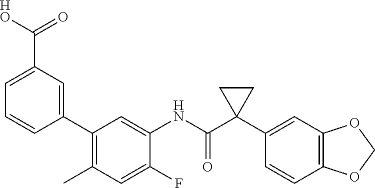 [SMILES: Cc1cc(F)c(NC(=O)C2(c3ccc4c(c3)OCO4)CC2)cc1-c1cccc(C(=O)O)c1] | 640 |
| 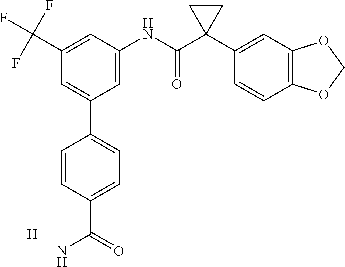 | 641 |
| 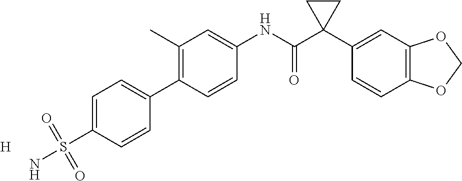 | 642 |
| 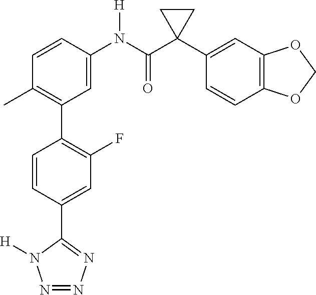 [SMILES: Cc1ccc(NC(=O)C2(c3ccc4c(c3)OCO4)CC2)cc1-c1ccc(-c2nnn[nH]2)cc1F] | 643 |
| 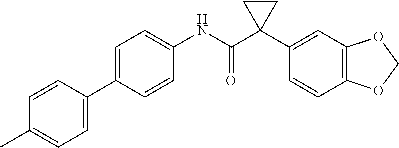 [SMILES: Cc1ccc(-c2ccc(NC(=O)C3(c4ccc5c(c4)OCO5)CC3)cc2)cc1] | 644 |
| 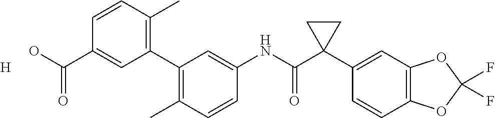 | 645 |
| 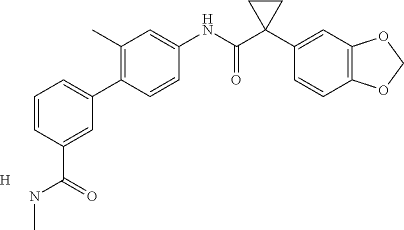 | 646 |
| 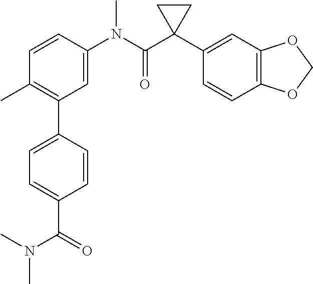 [SMILES: Cc1ccc(N(C)C(=O)C2(c3ccc4c(c3)OCO4)CC2)cc1-c1ccc(C(=O)N(C)C)cc1] | 647 |
| 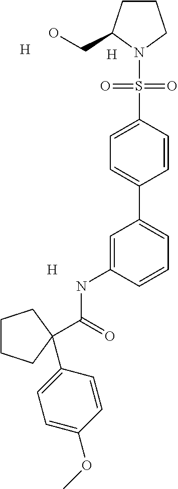 | 648 |
| 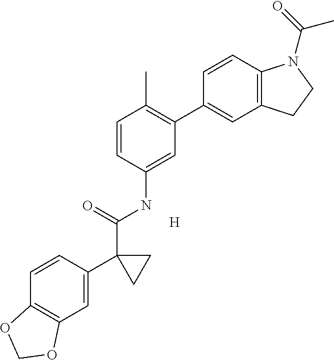 | 649 |
| 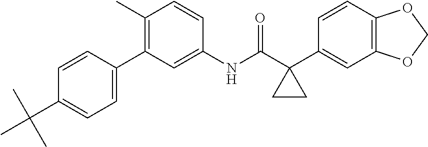 [SMILES: Cc1ccc(NC(=O)C2(c3ccc4c(c3)OCO4)CC2)cc1-c1ccc(C(C)(C)C)cc1] | 650 |
| 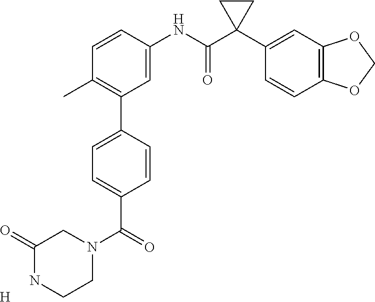 | 651 |
| 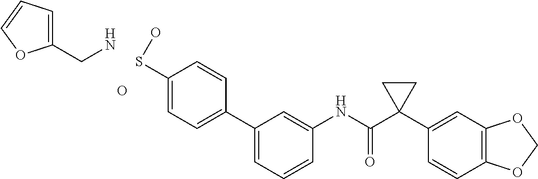 | 652 |
| 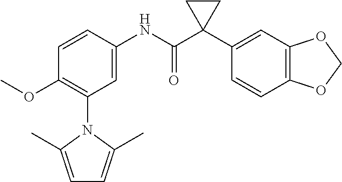 [SMILES: COc1ccc(NC(=O)C2(c3ccc4c(c3)OCO4)CC2)cc1-n1c(C)ccc1C] | 653 |
| 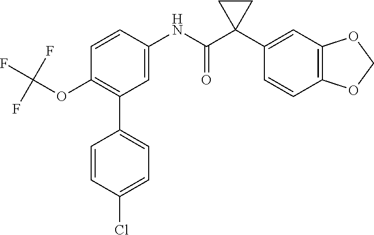 [SMILES: O=C(Nc1ccc(OC(F)(F)F)c(-c2ccc(Cl)cc2)c1)C1(c2ccc3c(c2)OCO3)CC1] | 654 |
| 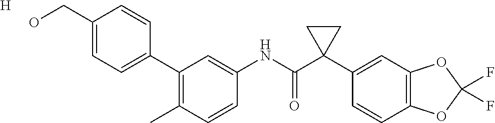 | 655 |
| 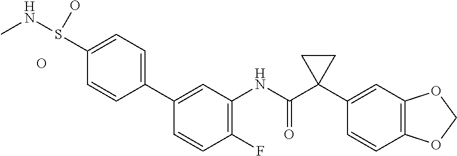 | 656 |
| 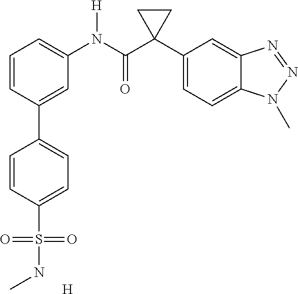 | 657 |
| 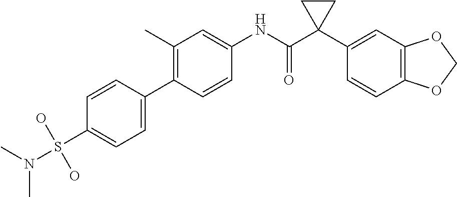 | 658 |
| 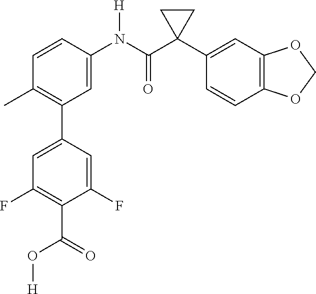 [SMILES: Cc1ccc(NC(=O)C2(c3ccc4c(c3)OCO4)CC2)cc1-c1cc(F)c(C(=O)O)c(F)c1] | 659 |
| 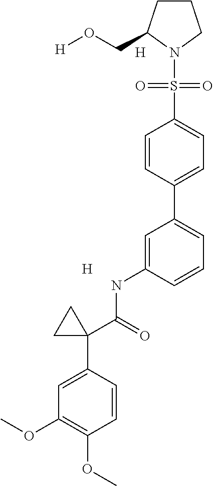 | 660 |
| 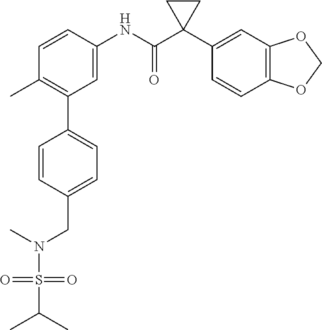 [SMILES: Cc1ccc(NC(=O)C2(c3ccc4c(c3)OCO4)CC2)cc1-c1ccc(CN(C)S(=O)(=O)C(C)C)cc1] | 661 |
| 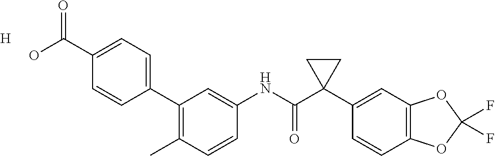 | 662 |
| 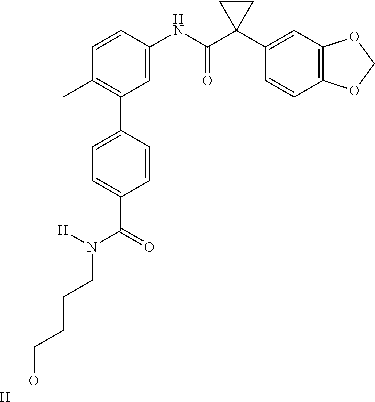 | 663 |
| 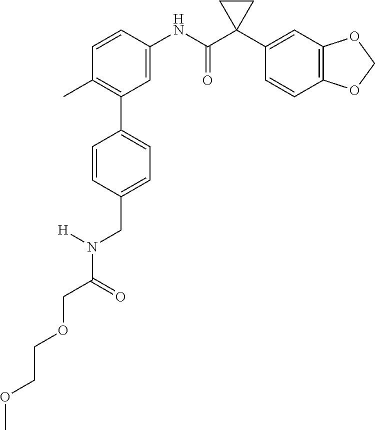 [SMILES: COCCOCC(=O)NCc1ccc(-c2cc(NC(=O)C3(c4ccc5c(c4)OCO5)CC3)ccc2C)cc1] | 664 |
| 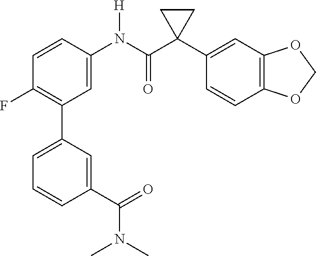 [SMILES: CN(C)C(=O)c1cccc(-c2cc(NC(=O)C3(c4ccc5c(c4)OCO5)CC3)ccc2F)c1] | 665 |
| 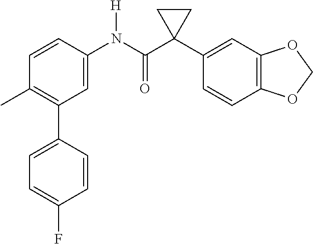 [SMILES: Cc1ccc(NC(=O)C2(c3ccc4c(c3)OCO4)CC2)cc1-c1ccc(F)cc1] | 666 |
| 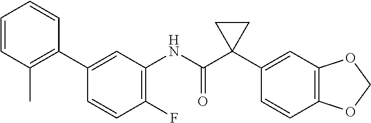 [SMILES: Cc1ccccc1-c1ccc(F)c(NC(=O)C2(c3ccc4c(c3)OCO4)CC2)c1] | 667 |
| 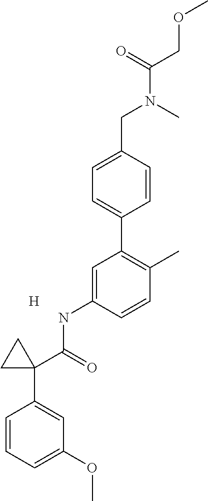 | 668 |
| 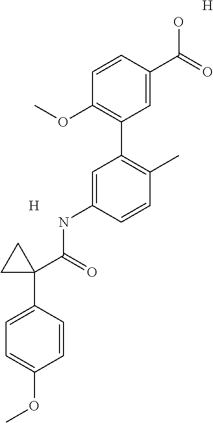 | 669 |
| 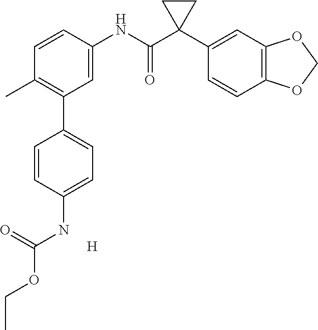 | 670 |
| 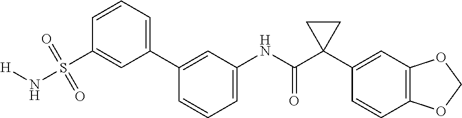 [SMILES: NS(=O)(=O)c1cccc(-c2cccc(NC(=O)C3(c4ccc5c(c4)OCO5)CC3)c2)c1] | 671 |
| 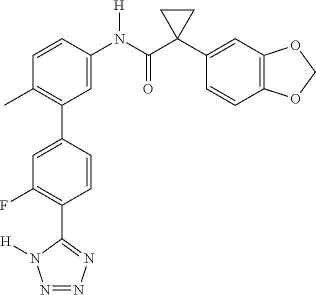 [SMILES: Cc1ccc(NC(=O)C2(c3ccc4c(c3)OCO4)CC2)cc1-c1ccc(-c2nnn[nH]2)c(F)c1] | 672 |
| 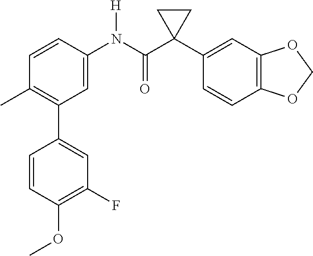 [SMILES: COc1ccc(-c2cc(NC(=O)C3(c4ccc5c(c4)OCO5)CC3)ccc2C)cc1F] | 673 |
| 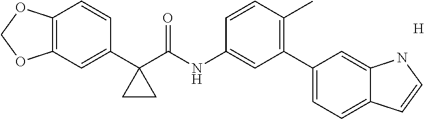 | 674 |
| 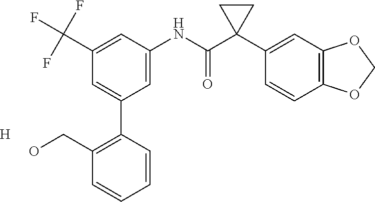 | 675 |
| 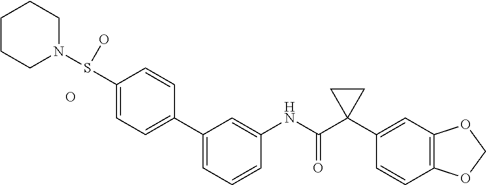 | 676 |
| 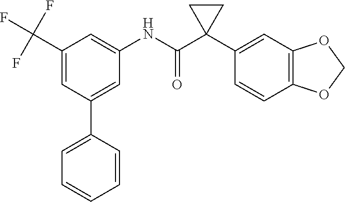 [SMILES: O=C(Nc1cc(-c2ccccc2)cc(C(F)(F)F)c1)C1(c2ccc3c(c2)OCO3)CC1] | 677 |
| 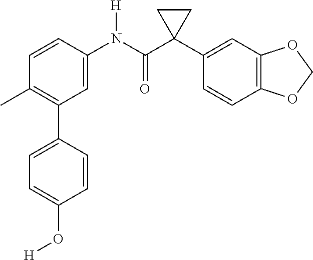 [SMILES: Cc1ccc(NC(=O)C2(c3ccc4c(c3)OCO4)CC2)cc1-c1ccc(O)cc1] | 678 |
| 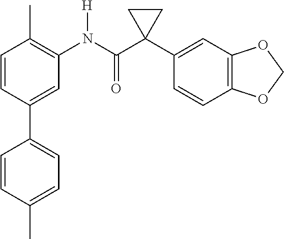 [SMILES: Cc1ccc(-c2ccc(C)c(NC(=O)C3(c4ccc5c(c4)OCO5)CC3)c2)cc1] | 679 |
| 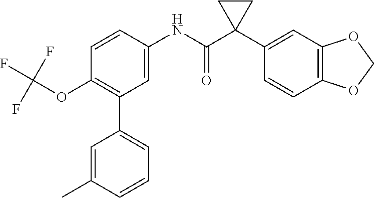 [SMILES: Cc1cccc(-c2cc(NC(=O)C3(c4ccc5c(c4)OCO5)CC3)ccc2OC(F)(F)F)c1] | 680 |
| 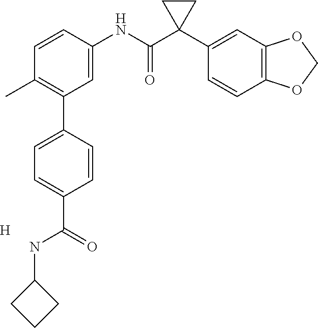 | 681 |
| 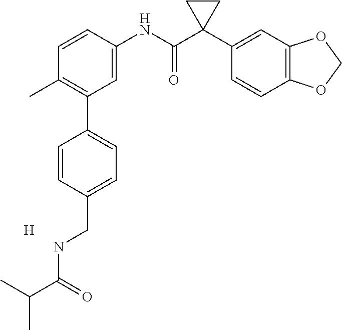 | 682 |
| 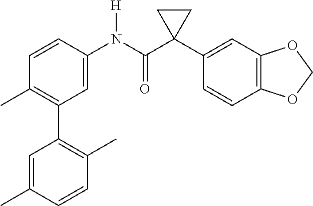 [SMILES: Cc1ccc(C)c(-c2cc(NC(=O)C3(c4ccc5c(c4)OCO5)CC3)ccc2C)c1] | 683 |
| 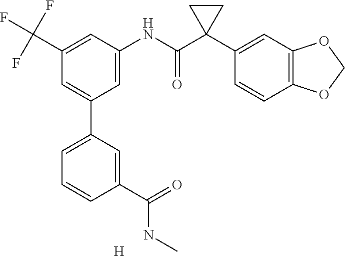 | 684 |
| 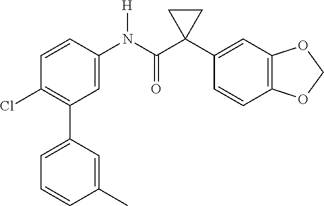 [SMILES: Cc1cccc(-c2cc(NC(=O)C3(c4ccc5c(c4)OCO5)CC3)ccc2Cl)c1] | 685 |
| 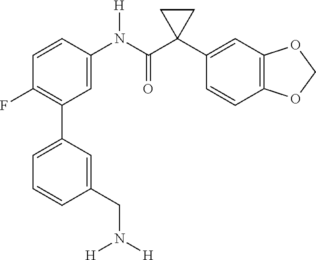 [SMILES: NCc1cccc(-c2cc(NC(=O)C3(c4ccc5c(c4)OCO5)CC3)ccc2F)c1] | 686 |
| 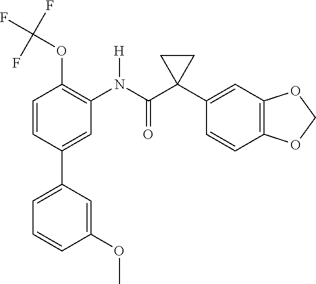 [SMILES: COc1cccc(-c2ccc(OC(F)(F)F)c(NC(=O)C3(c4ccc5c(c4)OCO5)CC3)c2)c1] | 687 |
| 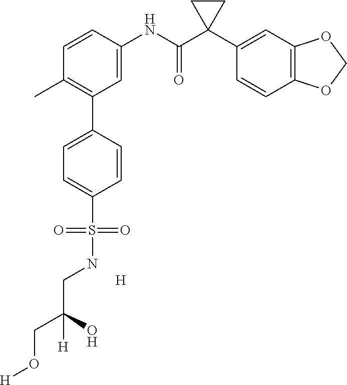 | 688 |
| 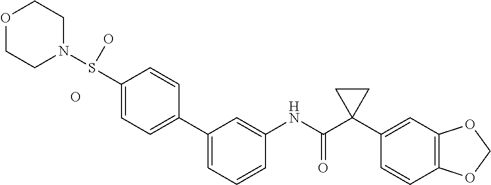 | 689 |
| 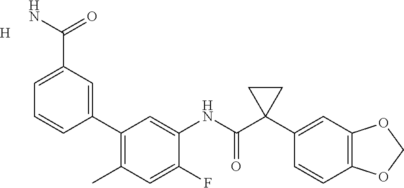 | 690 |
| 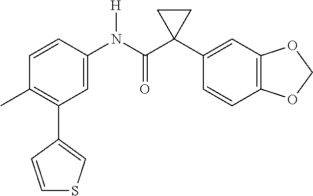 [SMILES: Cc1ccc(NC(=O)C2(c3ccc4c(c3)OCO4)CC2)cc1-c1ccsc1] | 691 |
| 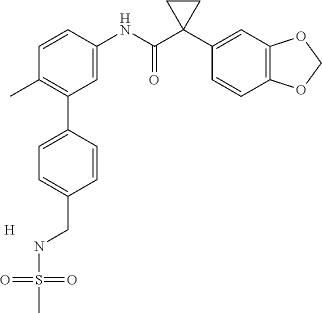 | 692 |
| 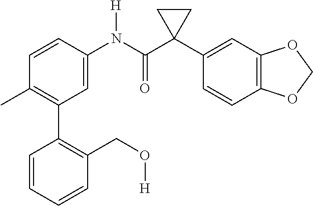 [SMILES: Cc1ccc(NC(=O)C2(c3ccc4c(c3)OCO4)CC2)cc1-c1ccccc1CO] | 693 |
| 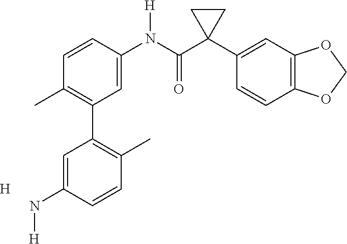 | 694 |
| 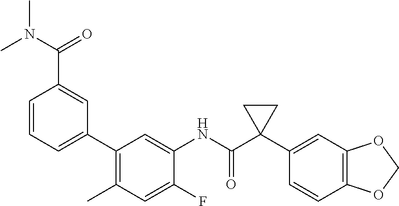 [SMILES: Cc1cc(F)c(NC(=O)C2(c3ccc4c(c3)OCO4)CC2)cc1-c1cccc(C(=O)N(C)C)c1] | 695 |
| 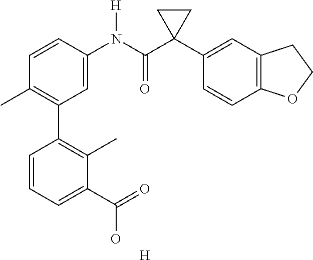 | 696 |
| 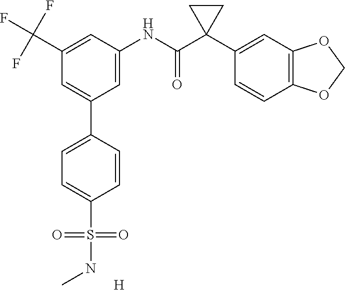 | 697 |
| 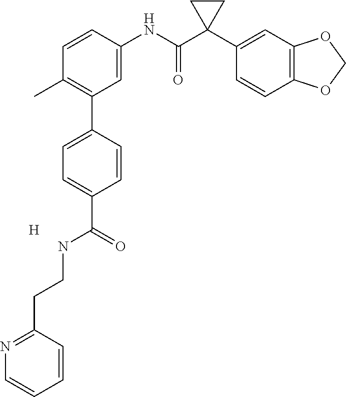 | 698 |
| 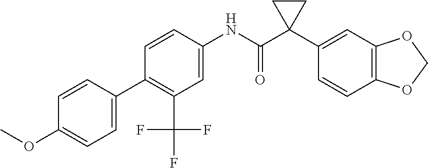 [SMILES: COc1ccc(-c2ccc(NC(=O)C3(c4ccc5c(c4)OCO5)CC3)cc2C(F)(F)F)cc1] | 699 |
| 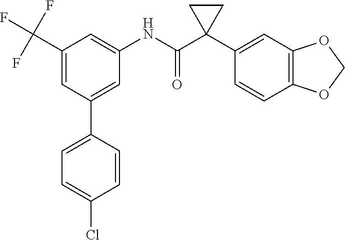 [SMILES: O=C(Nc1cc(-c2ccc(Cl)cc2)cc(C(F)(F)F)c1)C1(c2ccc3c(c2)OCO3)CC1] | 700 |
| 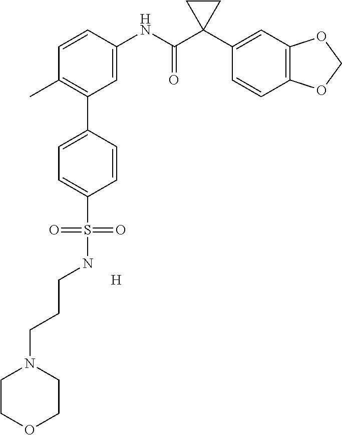 | 701 |
| 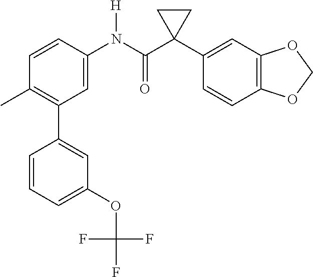 [SMILES: Cc1ccc(NC(=O)C2(c3ccc4c(c3)OCO4)CC2)cc1-c1cccc(OC(F)(F)F)c1] | 702 |
| 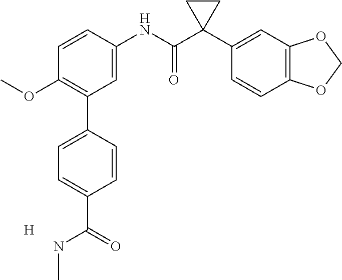 | 703 |
| 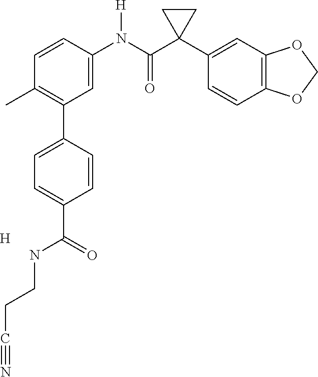 | 704 |
| 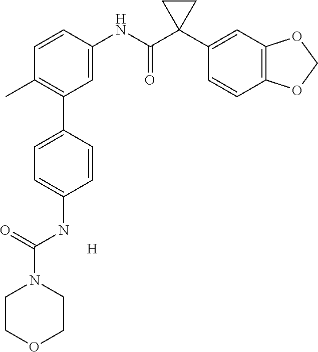 | 705 |
| 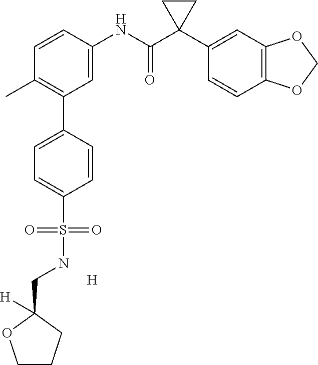 | 706 |
| 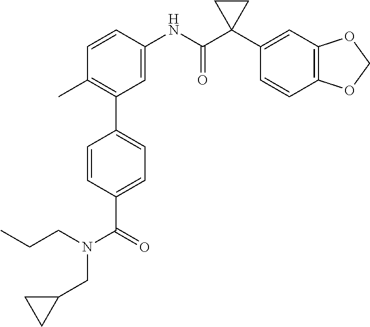 [SMILES: CCCN(CC1CC1)C(=O)c1ccc(-c2cc(NC(=O)C3(c4ccc5c(c4)OCO5)CC3)ccc2C)cc1] | 707 |
| 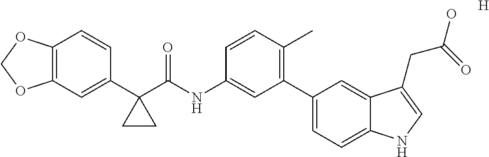 | 708 |
| 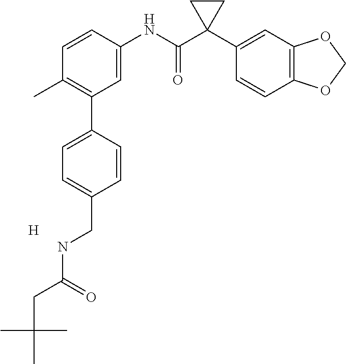 | 709 |
| 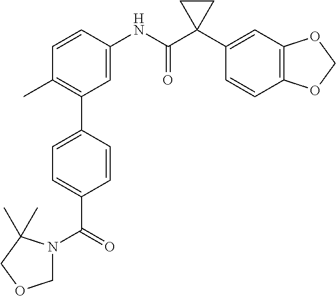 [SMILES: Cc1ccc(NC(=O)C2(c3ccc4c(c3)OCO4)CC2)cc1-c1ccc(C(=O)N2COCC2(C)C)cc1] | 710 |
| 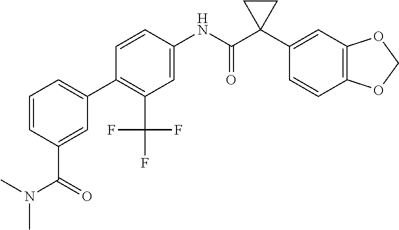 [SMILES: CN(C)C(=O)c1cccc(-c2ccc(NC(=O)C3(c4ccc5c(c4)OCO5)CC3)cc2C(F)(F)F)c1] | 711 |
| 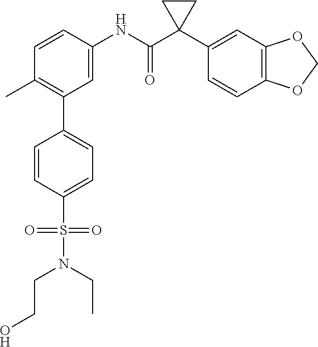 [SMILES: CCN(CCO)S(=O)(=O)c1ccc(-c2cc(NC(=O)C3(c4ccc5c(c4)OCO5)CC3)ccc2C)cc1] | 712 |
| 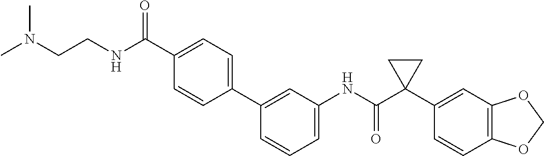 [SMILES: CN(C)CCNC(=O)c1ccc(-c2cccc(NC(=O)C3(c4ccc5c(c4)OCO5)CC3)c2)cc1] | 713 |
| 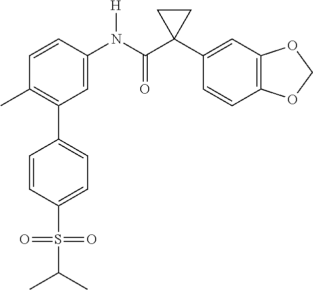 [SMILES: Cc1ccc(NC(=O)C2(c3ccc4c(c3)OCO4)CC2)cc1-c1ccc(S(=O)(=O)C(C)C)cc1] | 714 |
| 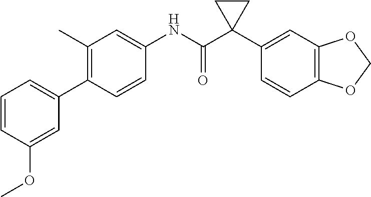 [SMILES: COc1cccc(-c2ccc(NC(=O)C3(c4ccc5c(c4)OCO5)CC3)cc2C)c1] | 715 |
| 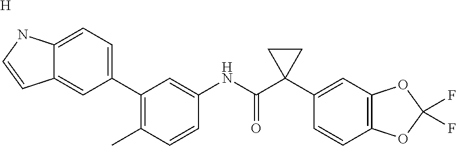 | 716 |
| 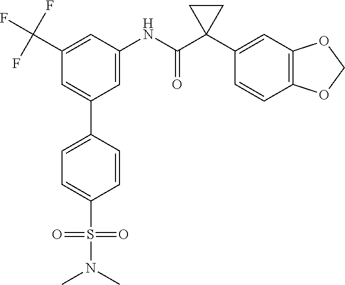 [SMILES: CN(C)S(=O)(=O)c1ccc(-c2cc(NC(=O)C3(c4ccc5c(c4)OCO5)CC3)cc(C(F)(F)F)c2)cc1] | 717 |
| 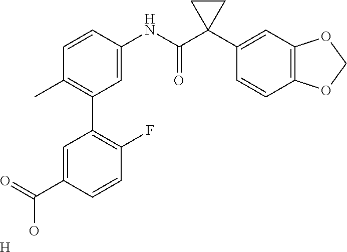 | 718 |
| 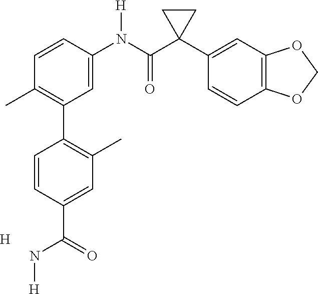 | 719 |
| 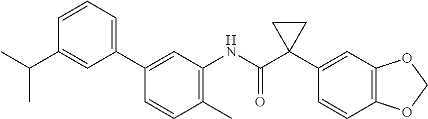 [SMILES: Cc1ccc(-c2cccc(C(C)C)c2)cc1NC(=O)C1(c2ccc3c(c2)OCO3)CC1] | 720 |
| 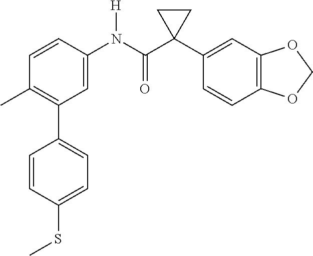 [SMILES: CSc1ccc(-c2cc(NC(=O)C3(c4ccc5c(c4)OCO5)CC3)ccc2C)cc1] | 721 |
| 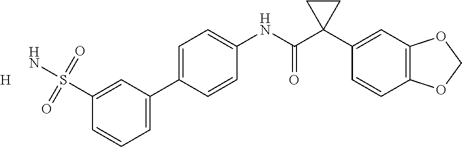 | 722 |
| 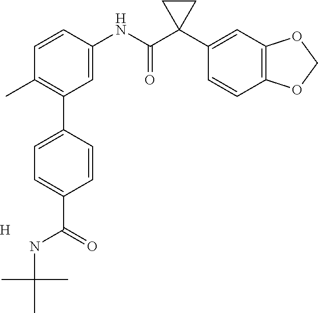 | 723 |
| 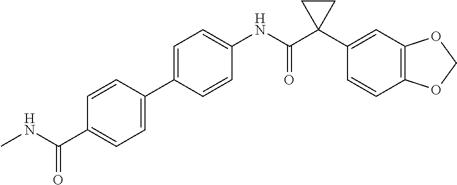 [SMILES: CNC(=O)c1ccc(-c2ccc(NC(=O)C3(c4ccc5c(c4)OCO5)CC3)cc2)cc1] | 724 |
| 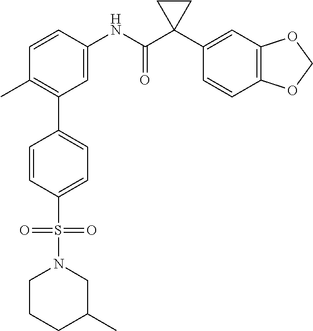 [SMILES: Cc1ccc(NC(=O)C2(c3ccc4c(c3)OCO4)CC2)cc1-c1ccc(S(=O)(=O)N2CCCC(C)C2)cc1] | 725 |
| 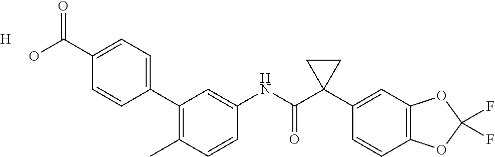 | 726 |
| 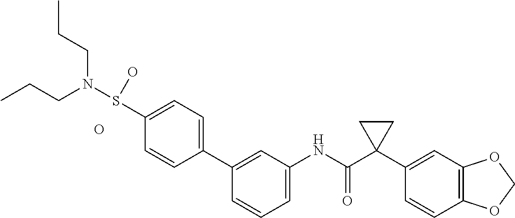 | 727 |
| 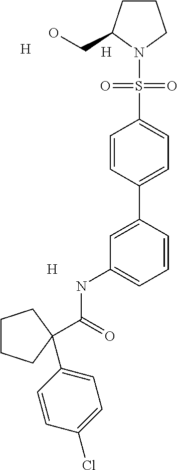 | 728 |
| 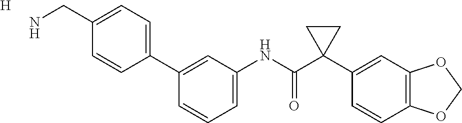 | 729 |
| 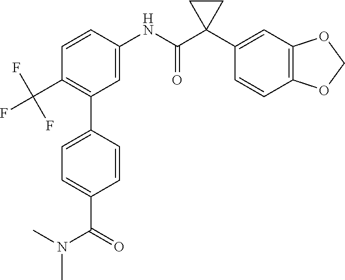 [SMILES: CN(C)C(=O)c1ccc(-c2cc(NC(=O)C3(c4ccc5c(c4)OCO5)CC3)ccc2C(F)(F)F)cc1] | 730 |
| 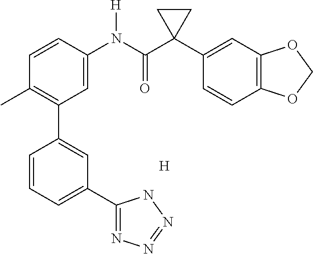 | 731 |
| 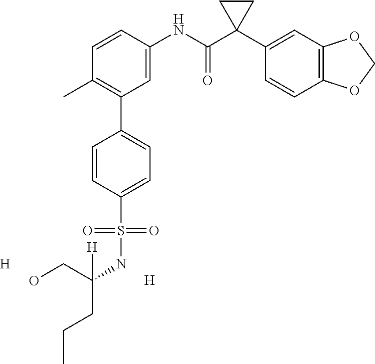 | 732 |
| 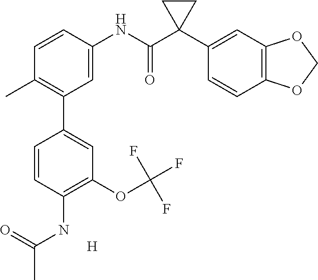 | 733 |
| 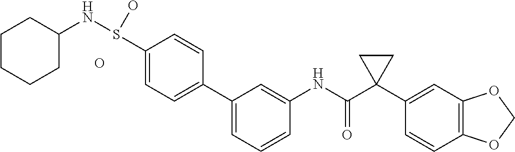 | 734 |
| 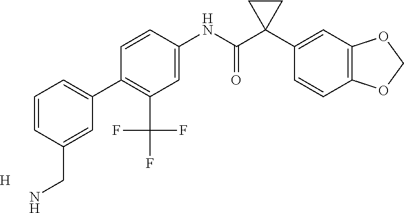 | 735 |
| 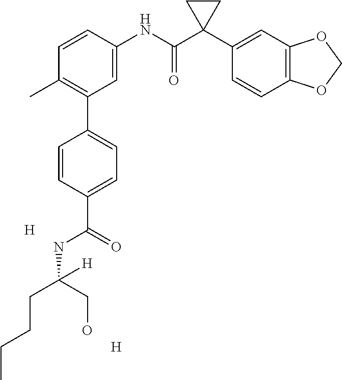 | 736 |
| 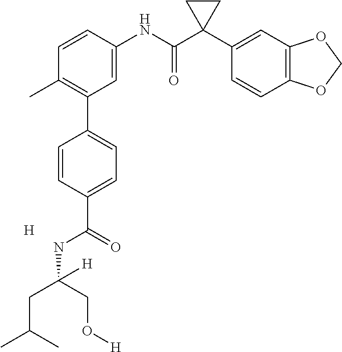 | 737 |
| 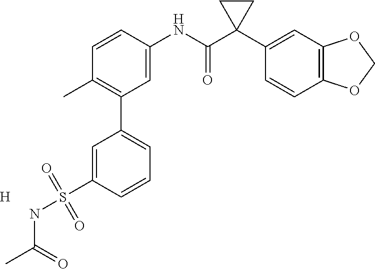 | 738 |
| 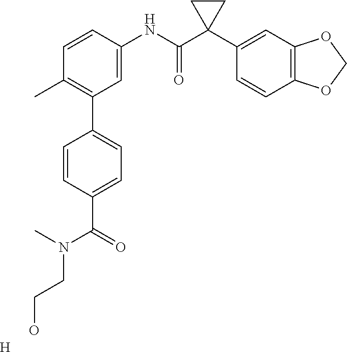 | 739 |
| 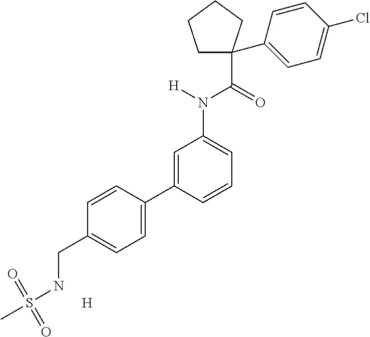 | 740 |
| 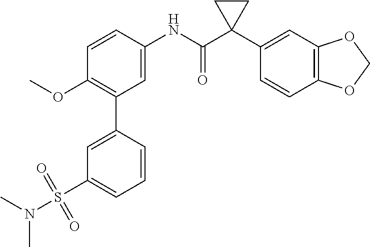 [SMILES: COc1ccc(NC(=O)C2(c3ccc4c(c3)OCO4)CC2)cc1-c1cccc(S(=O)(=O)N(C)C)c1] | 741 |
| 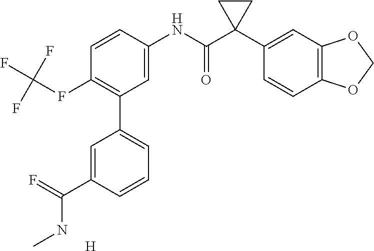 | 742 |
| 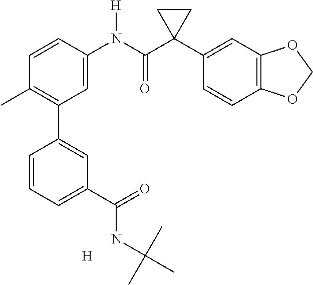 | 743 |
| 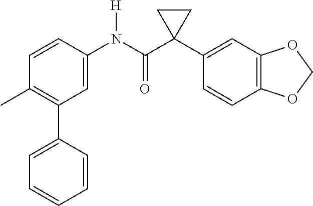 [SMILES: Cc1ccc(NC(=O)C2(c3ccc4c(c3)OCO4)CC2)cc1-c1ccccc1] | 744 |
| 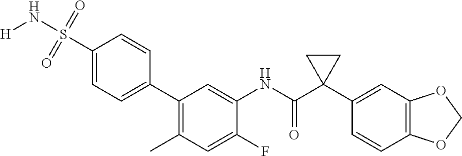 [SMILES: Cc1cc(F)c(NC(=O)C2(c3ccc4c(c3)OCO4)CC2)cc1-c1ccc(S(N)(=O)=O)cc1] | 745 |
| 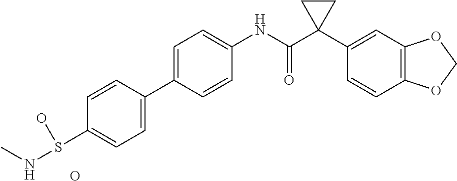 | 746 |
| 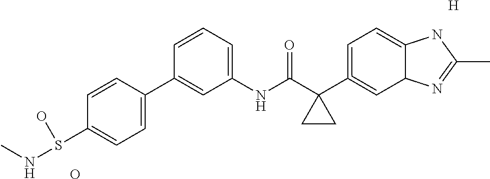 | 747 |
| 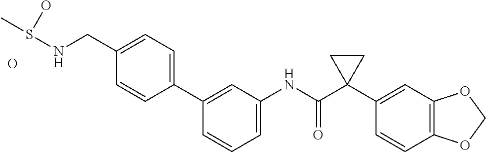 | 748 |
| 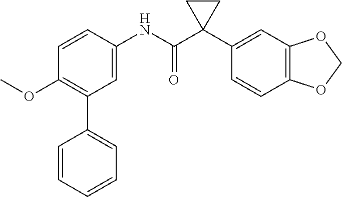 [SMILES: COc1ccc(NC(=O)C2(c3ccc4c(c3)OCO4)CC2)cc1-c1ccccc1] | 749 |
| 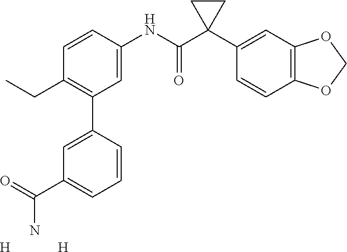 | 750 |
| 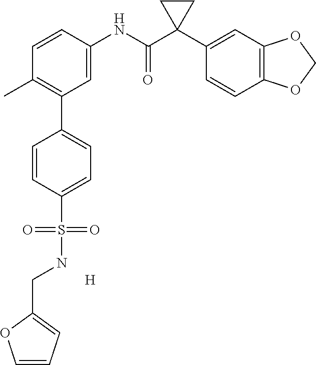 | 751 |
| 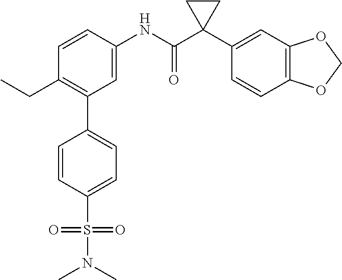 [SMILES: CCc1ccc(NC(=O)C2(c3ccc4c(c3)OCO4)CC2)cc1-c1ccc(S(=O)(=O)N(C)C)cc1] | 752 |
| 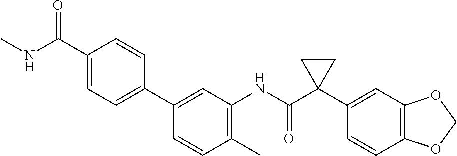 [SMILES: CNC(=O)c1ccc(-c2ccc(C)c(NC(=O)C3(c4ccc5c(c4)OCO5)CC3)c2)cc1] | 753 |
| 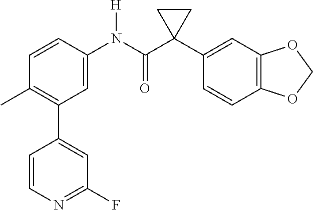 [SMILES: Cc1ccc(NC(=O)C2(c3ccc4c(c3)OCO4)CC2)cc1-c1ccnc(F)c1] | 754 |
| 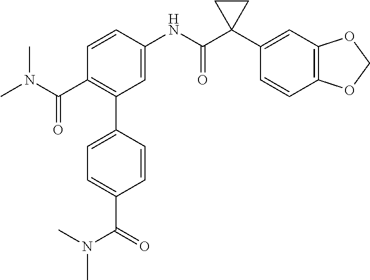 [SMILES: CN(C)C(=O)c1ccc(-c2cc(NC(=O)C3(c4ccc5c(c4)OCO5)CC3)ccc2C(=O)N(C)C)cc1] | 755 |
| 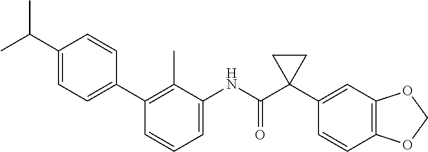 [SMILES: Cc1c(NC(=O)C2(c3ccc4c(c3)OCO4)CC2)cccc1-c1ccc(C(C)C)cc1] | 756 |
| 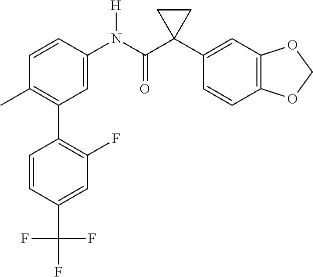 [SMILES: Cc1ccc(NC(=O)C2(c3ccc4c(c3)OCO4)CC2)cc1-c1ccc(C(F)(F)F)cc1F] | 757 |
| 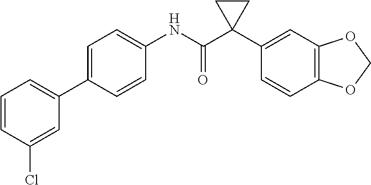 [SMILES: O=C(Nc1ccc(-c2cccc(Cl)c2)cc1)C1(c2ccc3c(c2)OCO3)CC1] | 758 |
| 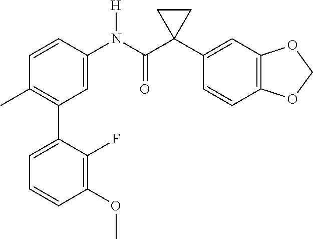 [SMILES: COc1cccc(-c2cc(NC(=O)C3(c4ccc5c(c4)OCO5)CC3)ccc2C)c1F] | 759 |
| 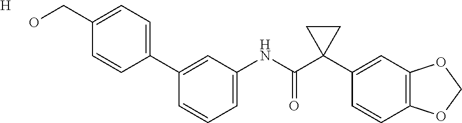 | 760 |
| 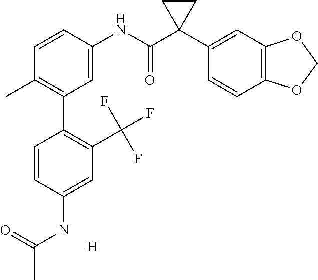 | 761 |
| 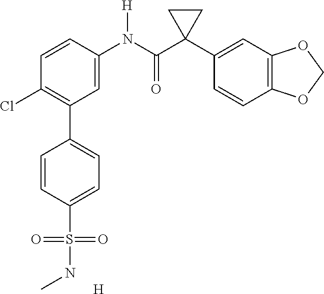 | 762 |
| 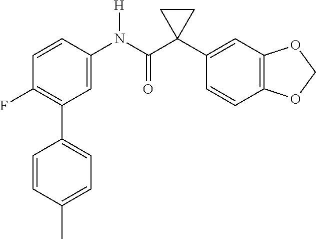 [SMILES: Cc1ccc(-c2cc(NC(=O)C3(c4ccc5c(c4)OCO5)CC3)ccc2F)cc1] | 763 |
| 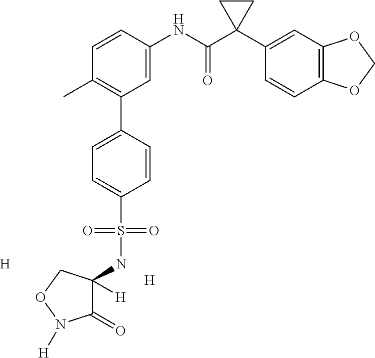 | 764 |
| 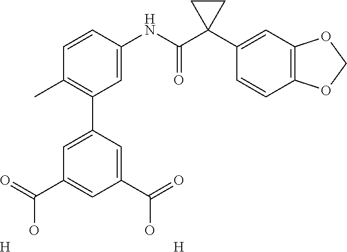 | 765 |
| 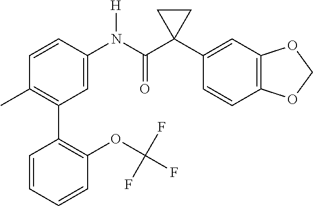 [SMILES: Cc1ccc(NC(=O)C2(c3ccc4c(c3)OCO4)CC2)cc1-c1ccccc1OC(F)(F)F] | 766 |
| 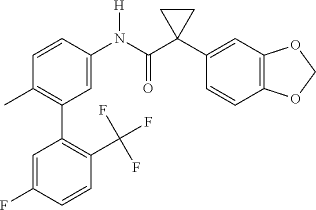 [SMILES: Cc1ccc(NC(=O)C2(c3ccc4c(c3)OCO4)CC2)cc1-c1cc(F)ccc1C(F)(F)F] | 767 |
| 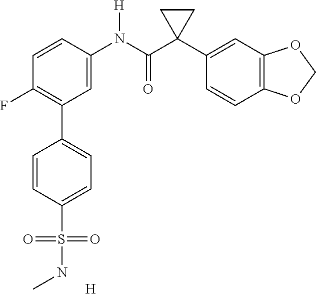 | 768 |
| 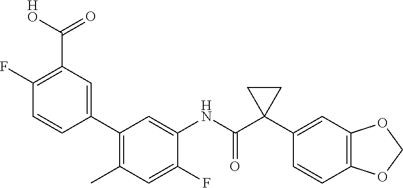 [SMILES: Cc1cc(F)c(NC(=O)C2(c3ccc4c(c3)OCO4)CC2)cc1-c1ccc(F)c(C(=O)O)c1] | 769 |
| 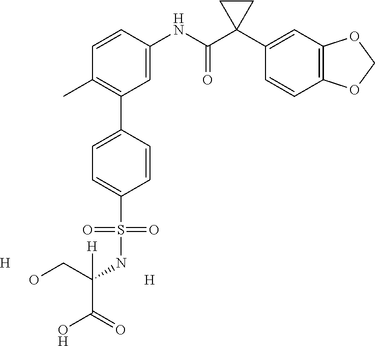 | 770 |
| 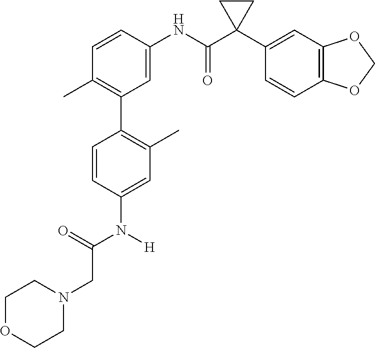 [SMILES: Cc1cc(NC(=O)CN2CCOCC2)ccc1-c1cc(NC(=O)C2(c3ccc4c(c3)OCO4)CC2)ccc1C] | 771 |
| 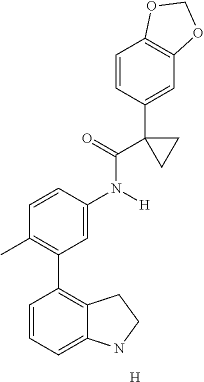 | 772 |
| 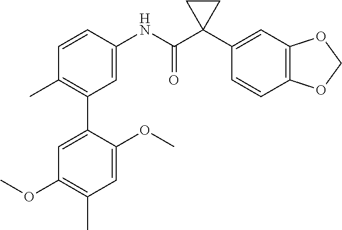 [SMILES: COc1cc(-c2cc(NC(=O)C3(c4ccc5c(c4)OCO5)CC3)ccc2C)c(OC)cc1C] | 773 |
| 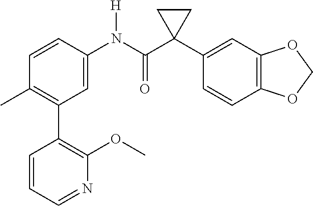 [SMILES: COc1ncccc1-c1cc(NC(=O)C2(c3ccc4c(c3)OCO4)CC2)ccc1C] | 774 |
| 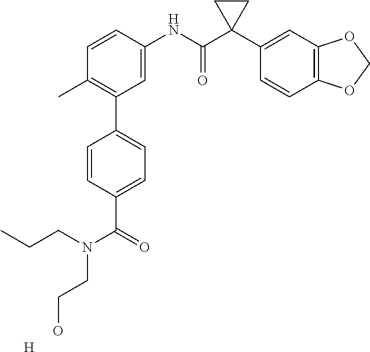 | 775 |
| 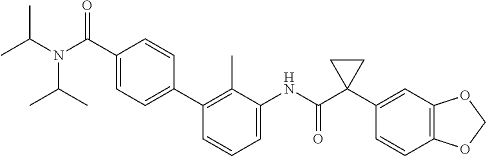 [SMILES: Cc1c(NC(=O)C2(c3ccc4c(c3)OCO4)CC2)cccc1-c1ccc(C(=O)N(C(C)C)C(C)C)cc1] | 776 |
| 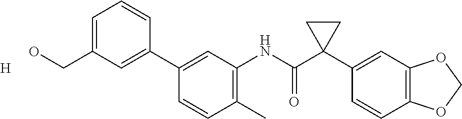 | 777 |
| 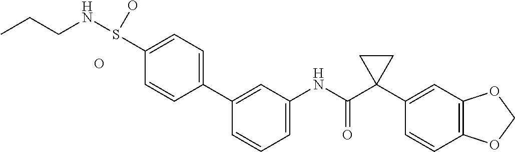 | 778 |
| 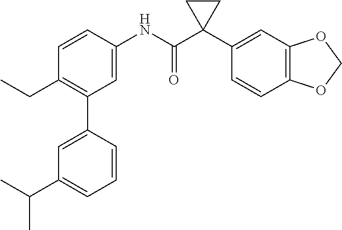 [SMILES: CCc1ccc(NC(=O)C2(c3ccc4c(c3)OCO4)CC2)cc1-c1cccc(C(C)C)c1] | 779 |
| 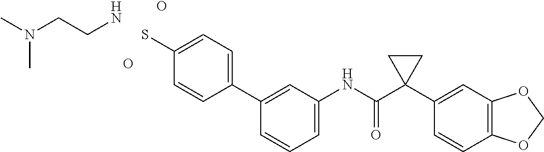 | 780 |
| 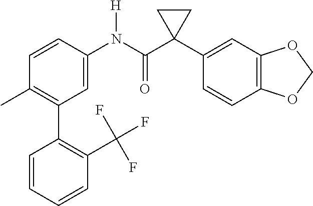 [SMILES: Cc1ccc(NC(=O)C2(c3ccc4c(c3)OCO4)CC2)cc1-c1ccccc1C(F)(F)F] | 781 |
| 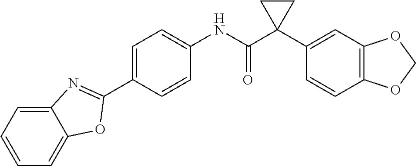 [SMILES: O=C(Nc1ccc(-c2nc3ccccc3o2)cc1)C1(c2ccc3c(c2)OCO3)CC1] | 782 |
| 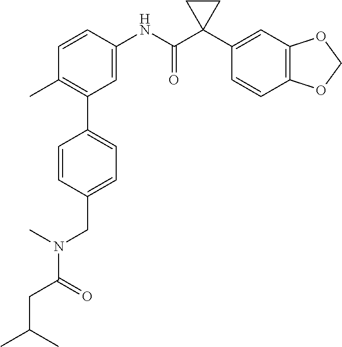 [SMILES: Cc1ccc(NC(=O)C2(c3ccc4c(c3)OCO4)CC2)cc1-c1ccc(CN(C)C(=O)CC(C)C)cc1] | 783 |
| 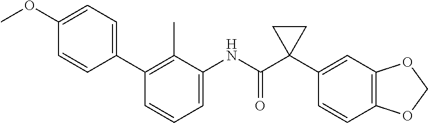 [SMILES: COc1ccc(-c2cccc(NC(=O)C3(c4ccc5c(c4)OCO5)CC3)c2C)cc1] | 784 |
| 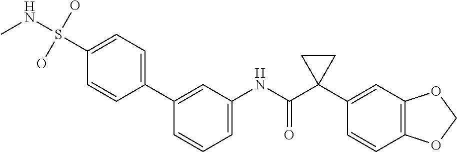 [SMILES: CNS(O)(O)c1ccc(-c2cccc(NC(=O)C3(c4ccc5c(c4)OCO5)CC3)c2)cc1] | 785 |
| 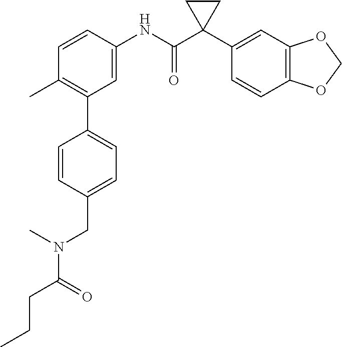 [SMILES: CCCC(=O)N(C)Cc1ccc(-c2cc(NC(=O)C3(c4ccc5c(c4)OCO5)CC3)ccc2C)cc1] | 786 |
| 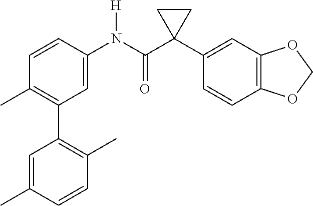 [SMILES: Cc1ccc(C)c(-c2cc(NC(=O)C3(c4ccc5c(c4)OCO5)CC3)ccc2C)c1] | 787 |
| 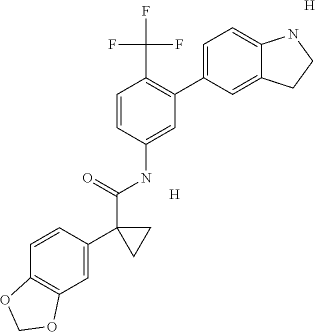 | 788 |
| 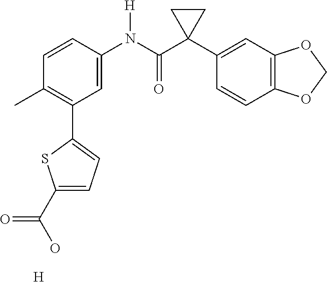 | 789 |
| 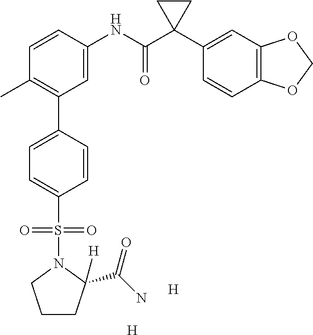 | 790 |
| 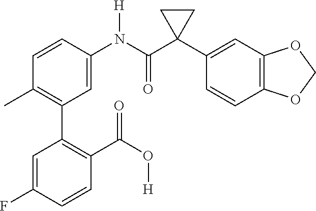 [SMILES: Cc1ccc(NC(=O)C2(c3ccc4c(c3)OCO4)CC2)cc1-c1cc(F)ccc1C(=O)O] | 791 |
| 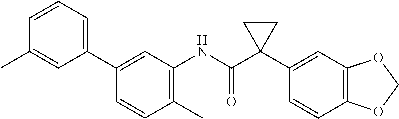 [SMILES: Cc1cccc(-c2ccc(C)c(NC(=O)C3(c4ccc5c(c4)OCO5)CC3)c2)c1] | 792 |
| 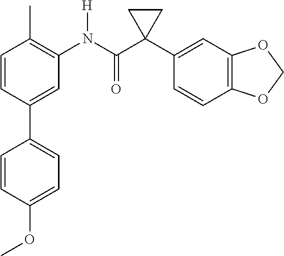 [SMILES: COc1ccc(-c2ccc(C)c(NC(=O)C3(c4ccc5c(c4)OCO5)CC3)c2)cc1] | 793 |
| 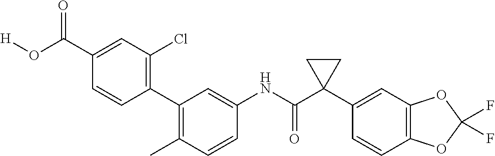 [SMILES: Cc1ccc(NC(=O)C2(c3ccc4c(c3)OC(F)(F)O4)CC2)cc1-c1ccc(C(=O)O)cc1Cl] | 794 |
| 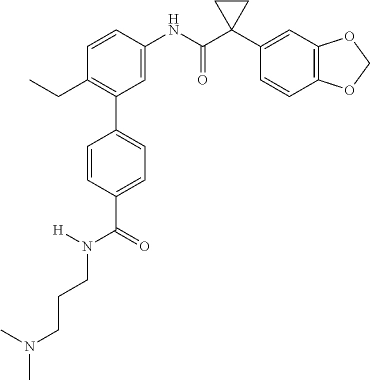 [SMILES: CCc1ccc(NC(=O)C2(c3ccc4c(c3)OCO4)CC2)cc1-c1ccc(C(=O)NCCCN(C)C)cc1] | 795 |
| 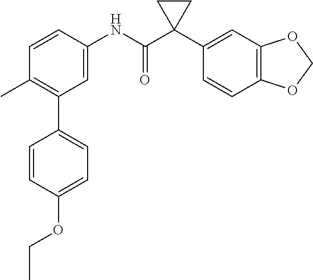 [SMILES: CCOc1ccc(-c2cc(NC(=O)C3(c4ccc5c(c4)OCO5)CC3)ccc2C)cc1] | 796 |
| 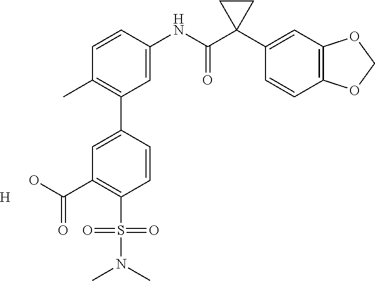 | 797 |
| 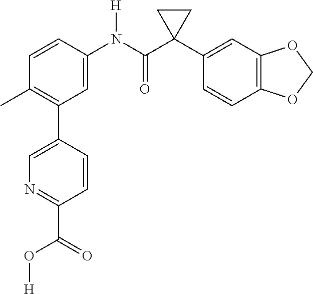 [SMILES: Cc1ccc(NC(=O)C2(c3ccc4c(c3)OCO4)CC2)cc1-c1ccc(C(=O)O)nc1] | 798 |
| 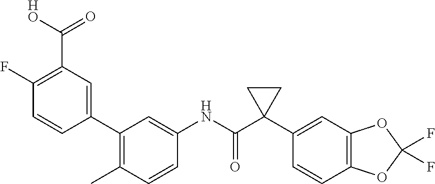 [SMILES: Cc1ccc(NC(=O)C2(c3ccc4c(c3)OC(F)(F)O4)CC2)cc1-c1ccc(F)c(C(=O)O)c1] | 799 |
| 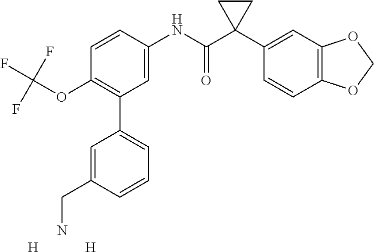 | 800 |
| 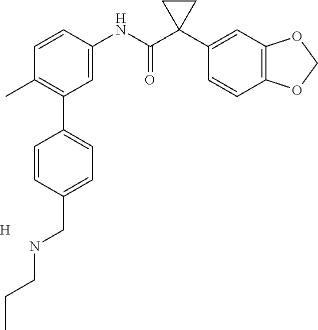 | 801 |
| 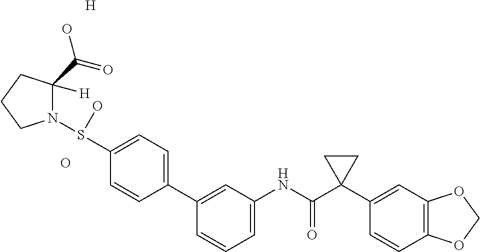 | 802 |
| 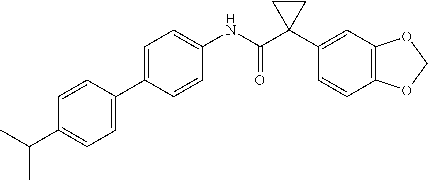 [SMILES: CC(C)c1ccc(-c2ccc(NC(=O)C3(c4ccc5c(c4)OCO5)CC3)cc2)cc1] | 803 |
| 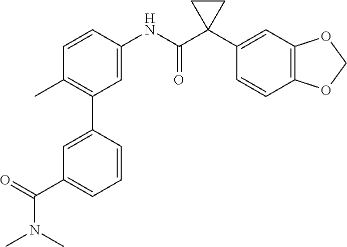 [SMILES: Cc1ccc(NC(=O)C2(c3ccc4c(c3)OCO4)CC2)cc1-c1cccc(C(=O)N(C)C)c1] | 804 |
| 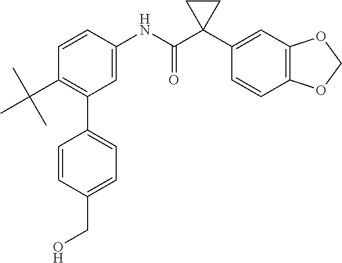 [SMILES: CC(C)(C)c1ccc(NC(=O)C2(c3ccc4c(c3)OCO4)CC2)cc1-c1ccc(CO)cc1] | 805 |
| 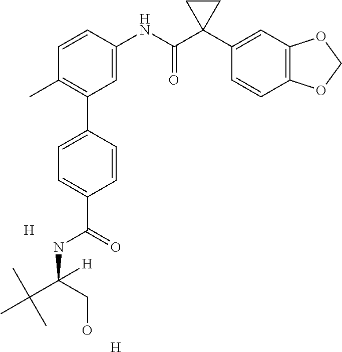 | 806 |
| 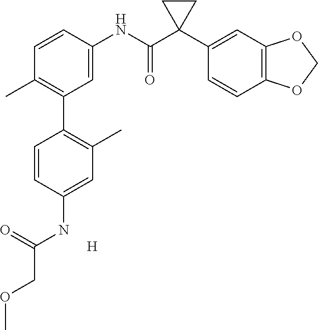 | 807 |
| 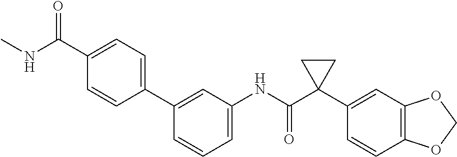 [SMILES: CNC(=O)c1ccc(-c2cccc(NC(=O)C3(c4ccc5c(c4)OCO5)CC3)c2)cc1] | 808 |
| 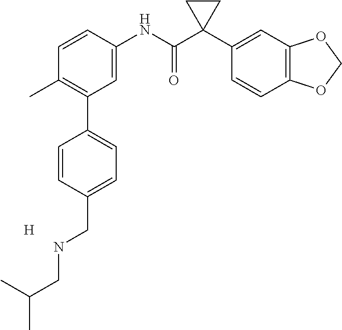 | 809 |
| 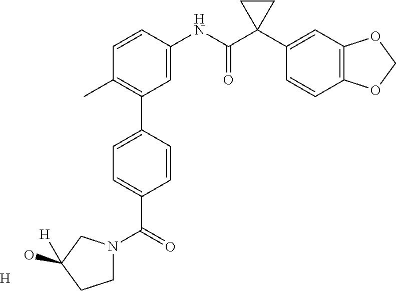 | 810 |
| 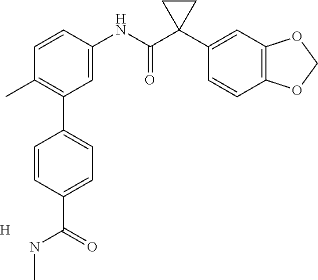 | 811 |
| 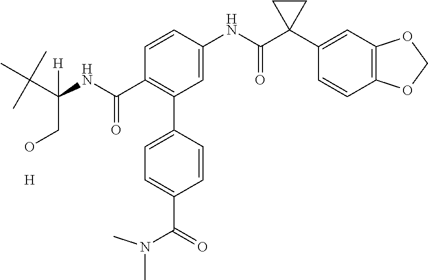 | 812 |
| 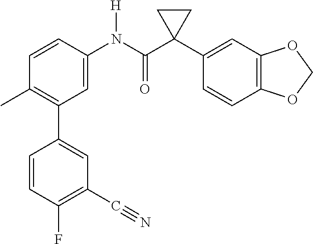 [SMILES: Cc1ccc(NC(=O)C2(c3ccc4c(c3)OCO4)CC2)cc1-c1ccc(F)c(C#N)c1] | 813 |
| 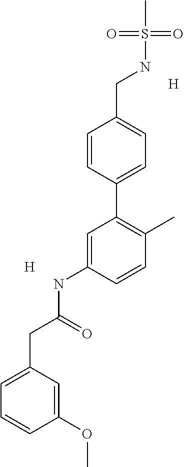 | 814 |
| 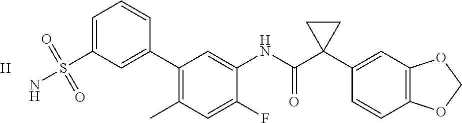 | 815 |
| 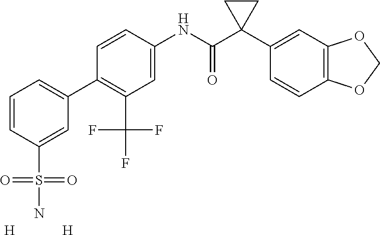 | 816 |
| 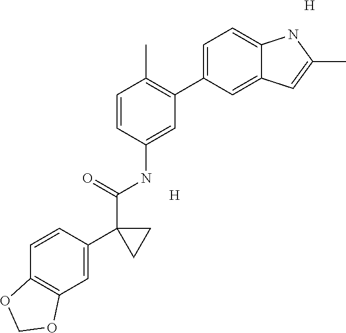 | 817 |
| 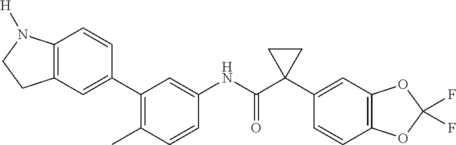 [SMILES: Cc1ccc(NC(=O)C2(c3ccc4c(c3)OC(F)(F)O4)CC2)cc1-c1ccc2c(c1)CCN2] | 818 |
| 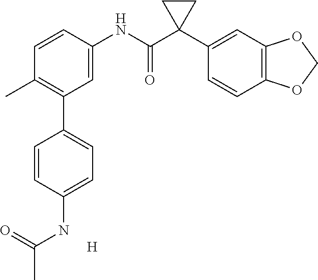 | 819 |
| 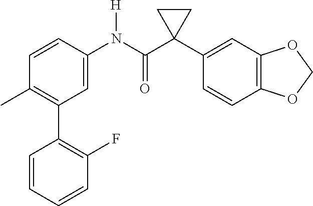 [SMILES: Cc1ccc(NC(=O)C2(c3ccc4c(c3)OCO4)CC2)cc1-c1ccccc1F] | 820 |
| 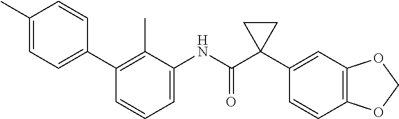 [SMILES: Cc1ccc(-c2cccc(NC(=O)C3(c4ccc5c(c4)OCO5)CC3)c2C)cc1] | 821 |
| 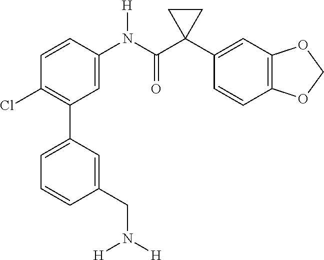 [SMILES: NCc1cccc(-c2cc(NC(=O)C3(c4ccc5c(c4)OCO5)CC3)ccc2Cl)c1] | 822 |
| 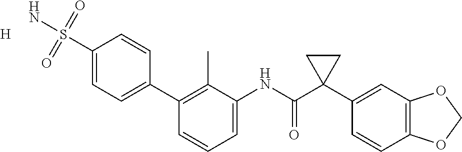 | 823 |
| 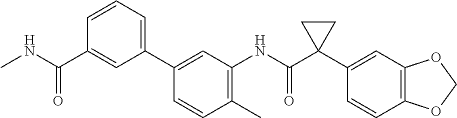 [SMILES: CNC(=O)c1cccc(-c2ccc(C)c(NC(=O)C3(c4ccc5c(c4)OCO5)CC3)c2)c1] | 824 |
| 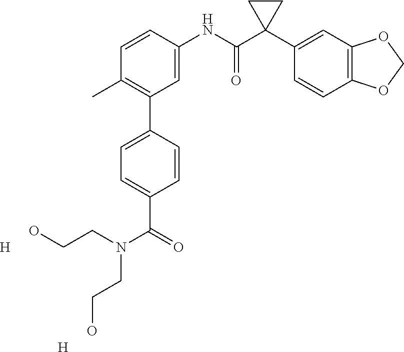 | 825 |
| 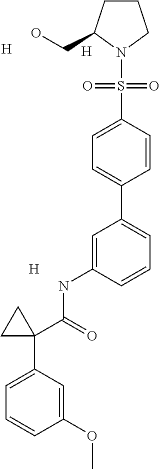 | 826 |
| 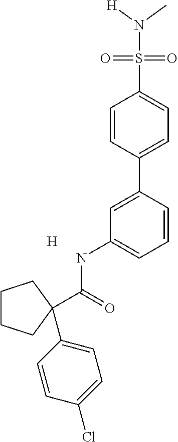 | 827 |
| 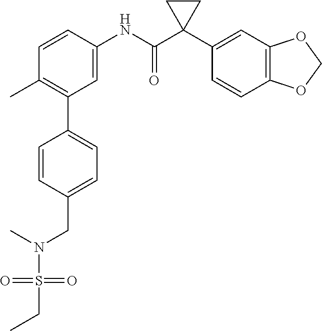 [SMILES: CCS(=O)(=O)N(C)Cc1ccc(-c2cc(NC(=O)C3(c4ccc5c(c4)OCO5)CC3)ccc2C)cc1] | 828 |
| 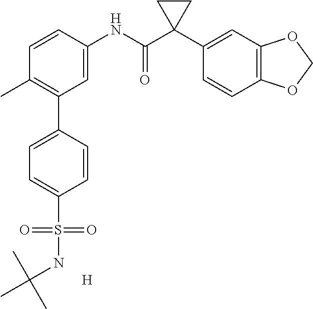 | 829 |
| 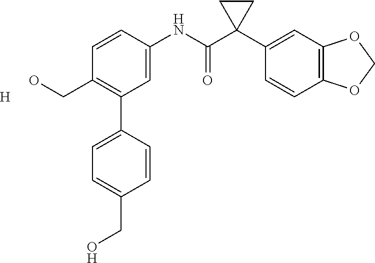 | 830 |
| 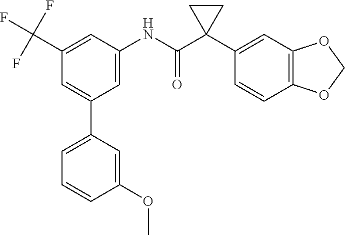 [SMILES: COc1cccc(-c2cc(NC(=O)C3(c4ccc5c(c4)OCO5)CC3)cc(C(F)(F)F)c2)c1] | 831 |
| 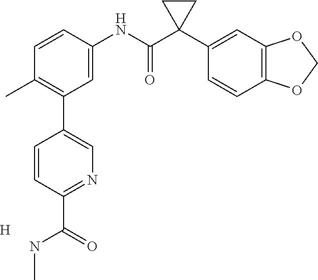 | 832 |
| 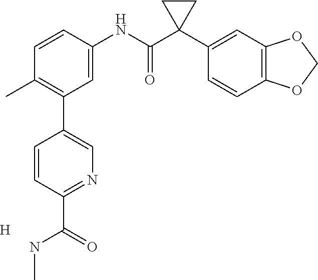 | 833 |
| 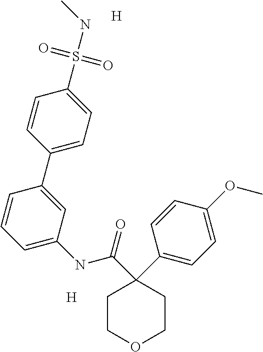 | 834 |
| 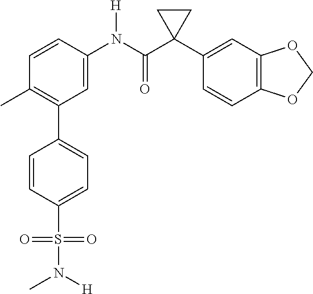 [SMILES: CNS(=O)(=O)c1ccc(-c2cc(NC(=O)C3(c4ccc5c(c4)OCO5)CC3)ccc2C)cc1] | 835 |
| 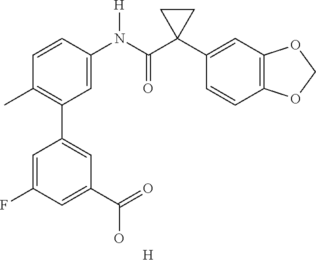 | 836 |
| 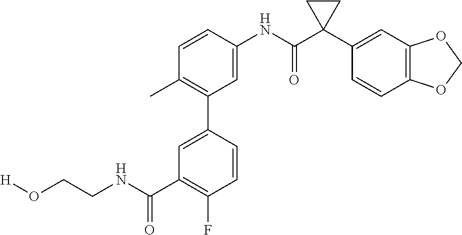 [SMILES: Cc1ccc(NC(=O)C2(c3ccc4c(c3)OCO4)CC2)cc1-c1ccc(F)c(C(=O)NCCO)c1] | 837 |
| 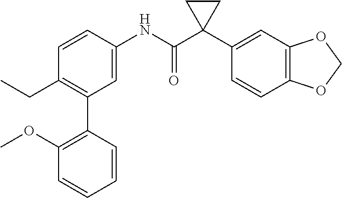 [SMILES: CCc1ccc(NC(=O)C2(c3ccc4c(c3)OCO4)CC2)cc1-c1ccccc1OC] | 838 |
| 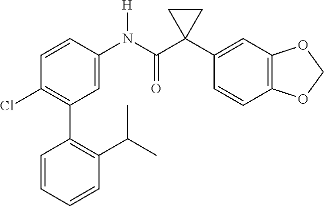 | 839 |
| 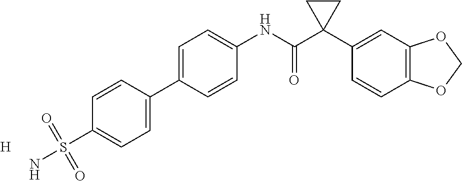 | 840 |
| 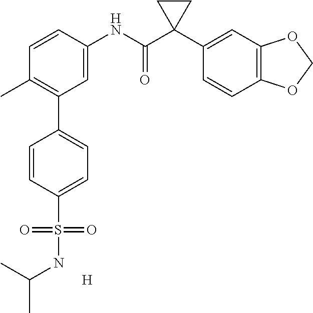 | 841 |
| 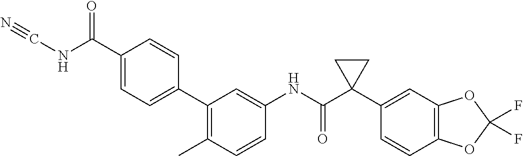 [SMILES: Cc1ccc(NC(=O)C2(c3ccc4c(c3)OC(F)(F)O4)CC2)cc1-c1ccc(C(=O)NC#N)cc1] | 842 |
| 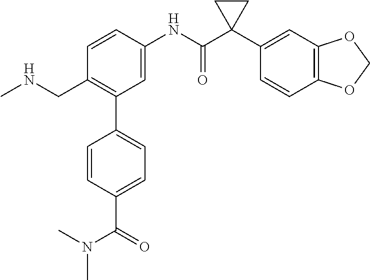 [SMILES: CNCc1ccc(NC(=O)C2(c3ccc4c(c3)OCO4)CC2)cc1-c1ccc(C(=O)N(C)C)cc1] | 843 |
| 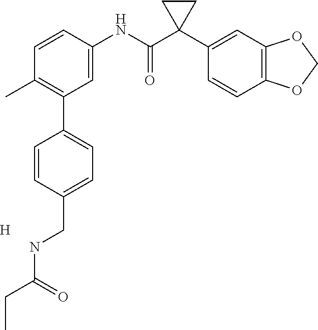 | 844 |
| 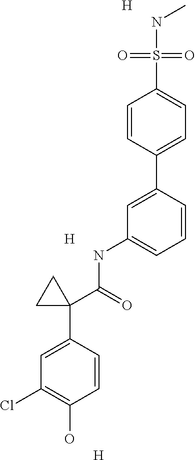 | 845 |
| 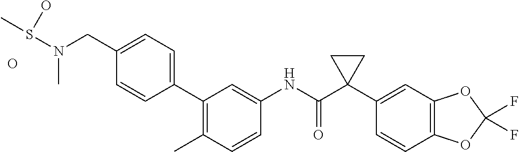 | 846 |
| 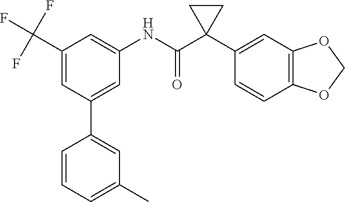 [SMILES: Cc1cccc(-c2cc(NC(=O)C3(c4ccc5c(c4)OCO5)CC3)cc(C(F)(F)F)c2)c1] | 847 |
| 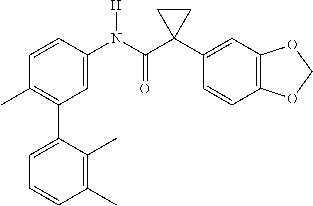 [SMILES: Cc1ccc(NC(=O)C2(c3ccc4c(c3)OCO4)CC2)cc1-c1cccc(C)c1C] | 848 |
| 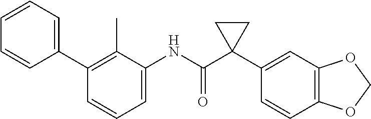 [SMILES: Cc1c(NC(=O)C2(c3ccc4c(c3)OCO4)CC2)cccc1-c1ccccc1] | 849 |
| 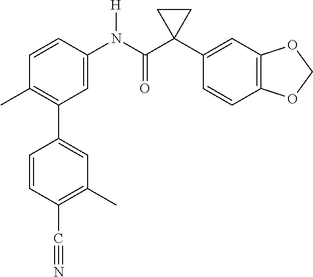 [SMILES: Cc1cc(-c2cc(NC(=O)C3(c4ccc5c(c4)OCO5)CC3)ccc2C)ccc1C#N] | 850 |
| 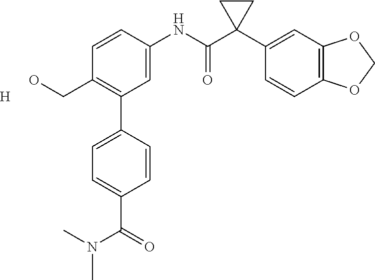 | 851 |
| 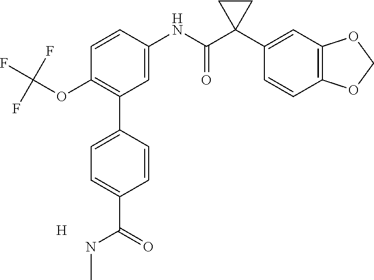 | 852 |
| 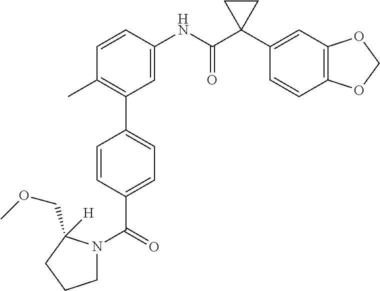 [SMILES: COC[C@H]1CCCN1C(=O)c1ccc(-c2cc(NC(=O)C3(c4ccc5c(c4)OCO5)CC3)ccc2C)cc1] | 853 |
| 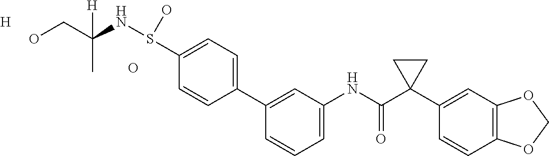 | 854 |
| 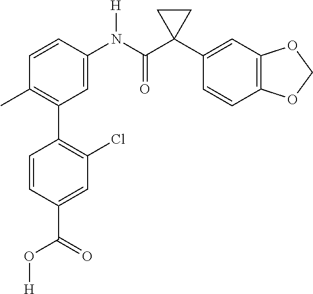 [SMILES: Cc1ccc(NC(=O)C2(c3ccc4c(c3)OCO4)CC2)cc1-c1ccc(C(=O)O)cc1Cl] | 855 |
| 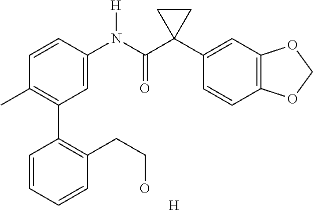 | 856 |
| 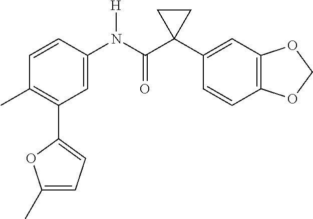 [SMILES: Cc1ccc(-c2cc(NC(=O)C3(c4ccc5c(c4)OCO5)CC3)ccc2C)o1] | 857 |
| 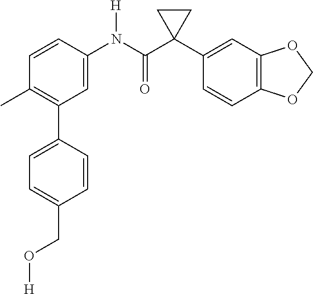 [SMILES: Cc1ccc(NC(=O)C2(c3ccc4c(c3)OCO4)CC2)cc1-c1ccc(CO)cc1] | 858 |
| 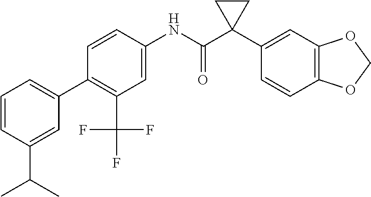 [SMILES: CC(C)c1cccc(-c2ccc(NC(=O)C3(c4ccc5c(c4)OCO5)CC3)cc2C(F)(F)F)c1] | 859 |
| 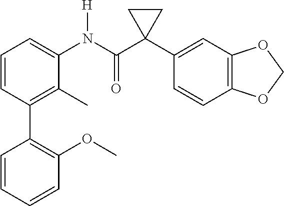 [SMILES: COc1ccccc1-c1cccc(NC(=O)C2(c3ccc4c(c3)OCO4)CC2)c1C] | 860 |
| 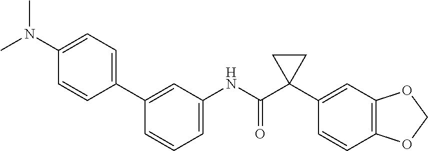 [SMILES: CN(C)c1ccc(-c2cccc(NC(=O)C3(c4ccc5c(c4)OCO5)CC3)c2)cc1] | 861 |
| 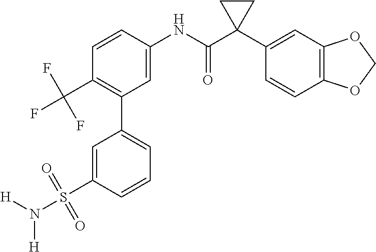 [SMILES: NS(=O)(=O)c1cccc(-c2cc(NC(=O)C3(c4ccc5c(c4)OCO5)CC3)ccc2C(F)(F)F)c1] | 862 |
| 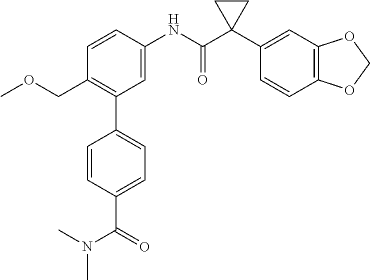 [SMILES: COCc1ccc(NC(=O)C2(c3ccc4c(c3)OCO4)CC2)cc1-c1ccc(C(=O)N(C)C)cc1] | 863 |
| 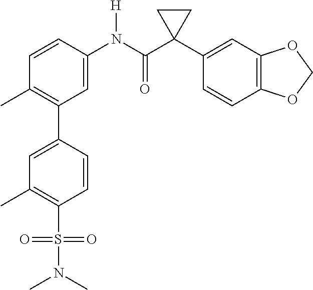 [SMILES: Cc1ccc(NC(=O)C2(c3ccc4c(c3)OCO4)CC2)cc1-c1ccc(S(=O)(=O)N(C)C)c(C)c1] | 864 |
| 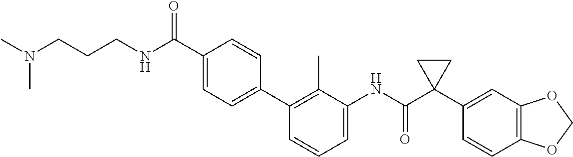 [SMILES: Cc1c(NC(=O)C2(c3ccc4c(c3)OCO4)CC2)cccc1-c1ccc(C(=O)NCCCN(C)C)cc1] | 865 |
| 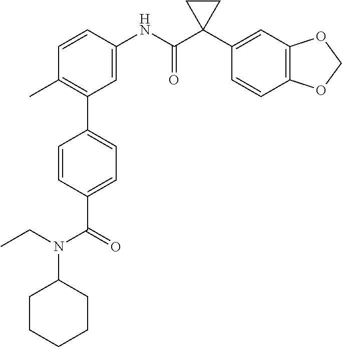 [SMILES: CCN(C(=O)c1ccc(-c2cc(NC(=O)C3(c4ccc5c(c4)OCO5)CC3)ccc2C)cc1)C1CCCCC1] | 866 |
| 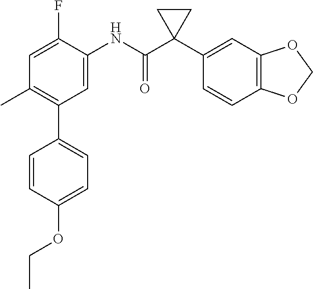 [SMILES: CCOc1ccc(-c2cc(NC(=O)C3(c4ccc5c(c4)OCO5)CC3)c(F)cc2C)cc1] | 867 |
| 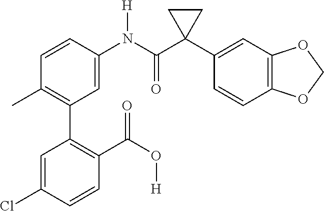 [SMILES: Cc1ccc(NC(=O)C2(c3ccc4c(c3)OCO4)CC2)cc1-c1cc(Cl)ccc1C(=O)O] | 868 |
| 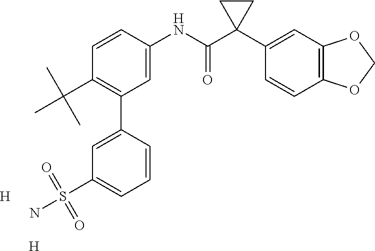 | 869 |
| 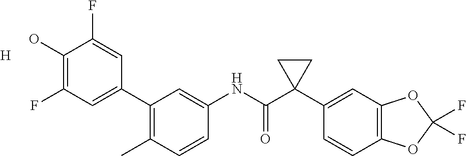 | 870 |
| 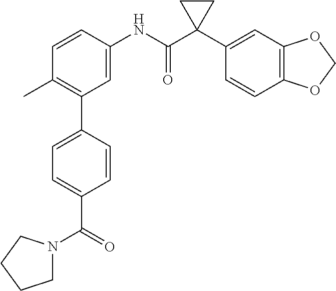 [SMILES: Cc1ccc(NC(=O)C2(c3ccc4c(c3)OCO4)CC2)cc1-c1ccc(C(=O)N2CCCC2)cc1] | 871 |
| 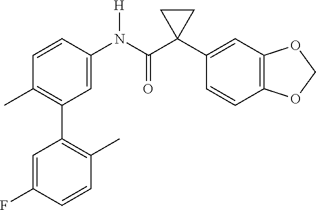 [SMILES: Cc1ccc(F)cc1-c1cc(NC(=O)C2(c3ccc4c(c3)OCO4)CC2)ccc1C] | 872 |
| 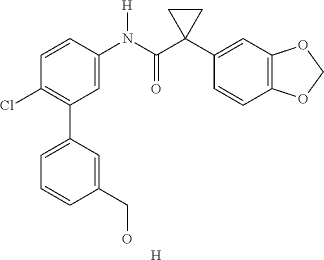 | 873 |
| 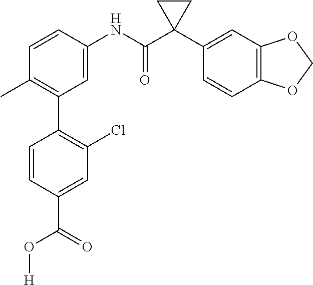 [SMILES: Cc1ccc(NC(=O)C2(c3ccc4c(c3)OCO4)CC2)cc1-c1ccc(C(=O)O)cc1Cl] | 874 |
| 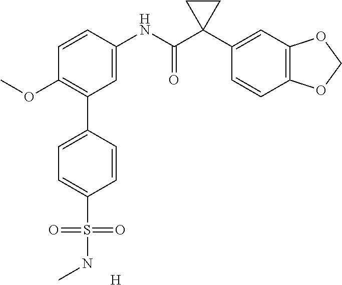 | 875 |
| 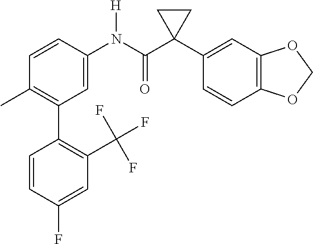 [SMILES: Cc1ccc(NC(=O)C2(c3ccc4c(c3)OCO4)CC2)cc1-c1ccc(F)cc1C(F)(F)F] | 876 |
| 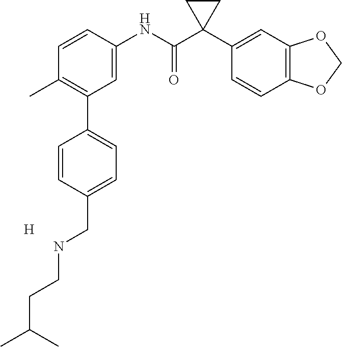 | 877 |
| 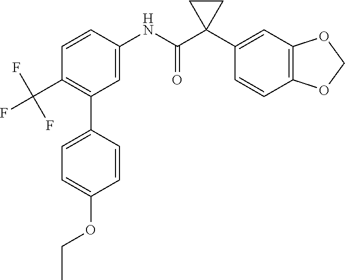 [SMILES: CCOc1ccc(-c2cc(NC(=O)C3(c4ccc5c(c4)OCO5)CC3)ccc2C(F)(F)F)cc1] | 878 |
| 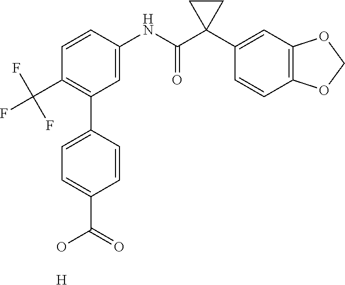 | 879 |
| 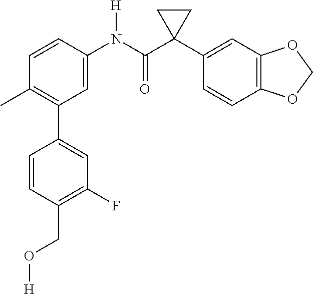 [SMILES: Cc1ccc(NC(=O)C2(c3ccc4c(c3)OCO4)CC2)cc1-c1ccc(CO)c(F)c1] | 880 |
| 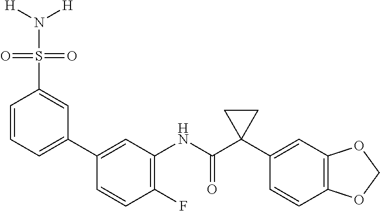 [SMILES: NS(=O)(=O)c1cccc(-c2ccc(F)c(NC(=O)C3(c4ccc5c(c4)OCO5)CC3)c2)c1] | 881 |
| 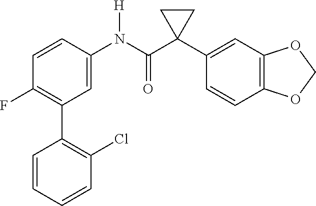 [SMILES: O=C(Nc1ccc(F)c(-c2ccccc2Cl)c1)C1(c2ccc3c(c2)OCO3)CC1] | 882 |
| 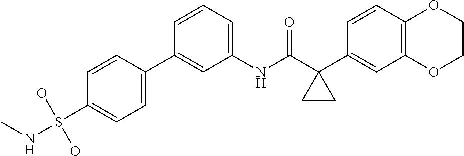 | 883 |
| 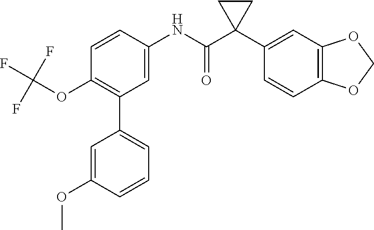 [SMILES: COc1cccc(-c2cc(NC(=O)C3(c4ccc5c(c4)OCO5)CC3)ccc2OC(F)(F)F)c1] | 884 |
| 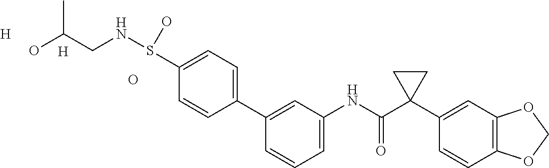 | 885 |
| 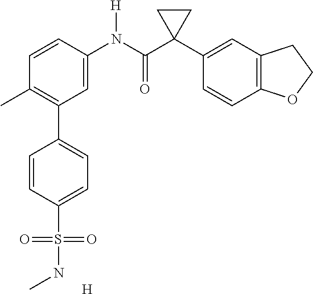 | 886 |
| 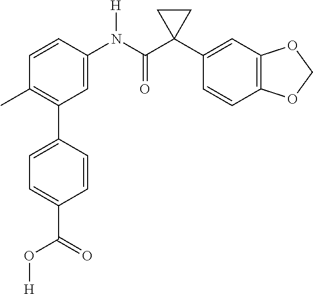 [SMILES: Cc1ccc(NC(=O)C2(c3ccc4c(c3)OCO4)CC2)cc1-c1ccc(C(=O)O)cc1] | 887 |
| 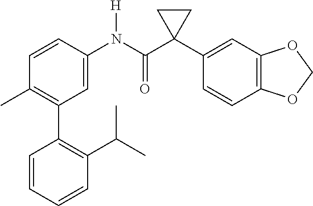 [SMILES: Cc1ccc(NC(=O)C2(c3ccc4c(c3)OCO4)CC2)cc1-c1ccccc1C(C)C] | 888 |
| 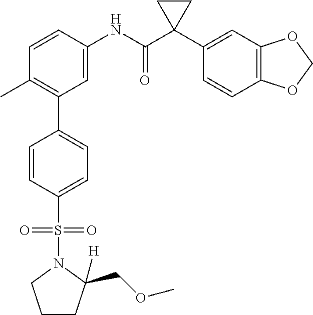 [SMILES: COC[C@H]1CCCN1S(=O)(=O)c1ccc(-c2cc(NC(=O)C3(c4ccc5c(c4)OCO5)CC3)ccc2C)cc1] | 889 |
| 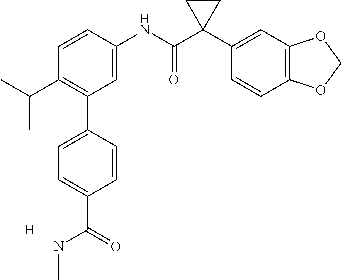 | 890 |
| 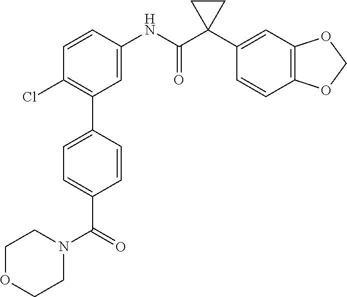 [SMILES: O=C(c1ccc(-c2cc(NC(=O)C3(c4ccc5c(c4)OCO5)CC3)ccc2Cl)cc1)N1CCOCC1] | 891 |
| 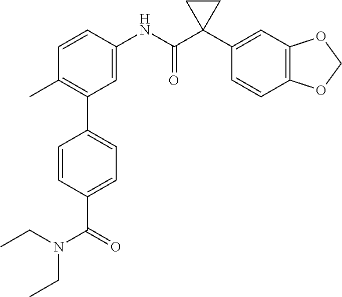 [SMILES: CCN(CC)C(=O)c1ccc(-c2cc(NC(=O)C3(c4ccc5c(c4)OCO5)CC3)ccc2C)cc1] | 892 |
| 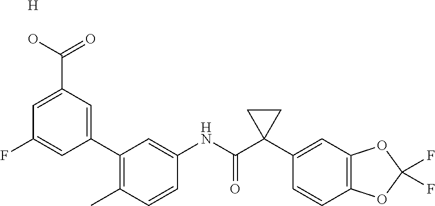 | 893 |
| 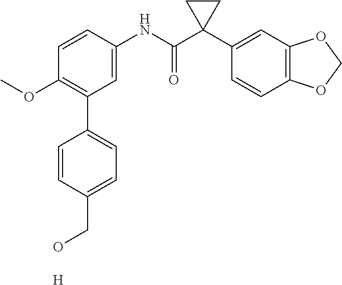 | 894 |
| 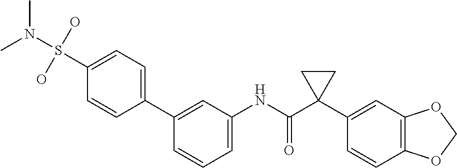 [SMILES: CN(C)S(O)(O)c1ccc(-c2cccc(NC(=O)C3(c4ccc5c(c4)OCO5)CC3)c2)cc1] | 895 |
| 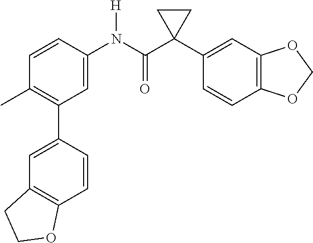 [SMILES: Cc1ccc(NC(=O)C2(c3ccc4c(c3)OCO4)CC2)cc1-c1ccc2c(c1)CCO2] | 896 |
| 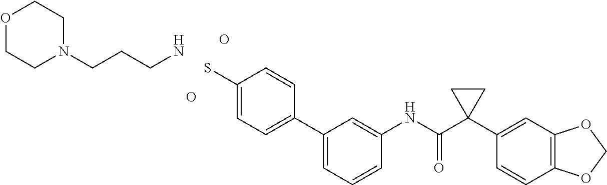 | 897 |
| 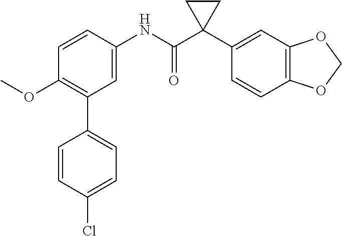 [SMILES: COc1ccc(NC(=O)C2(c3ccc4c(c3)OCO4)CC2)cc1-c1ccc(Cl)cc1] | 898 |
| 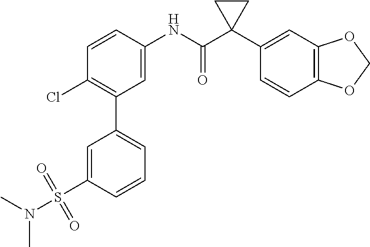 [SMILES: CN(C)S(=O)(=O)c1cccc(-c2cc(NC(=O)C3(c4ccc5c(c4)OCO5)CC3)ccc2Cl)c1] | 899 |
| 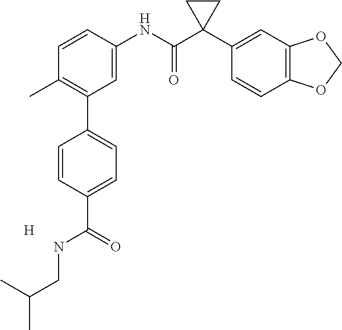 | 900 |
| 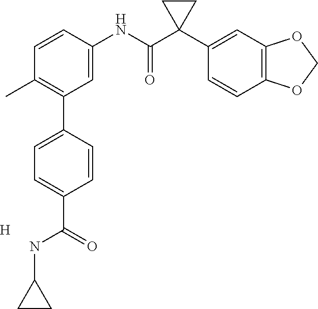 | 901 |
| 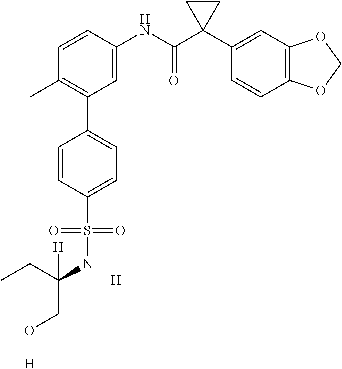 | 902 |
| 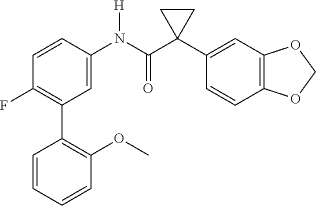 [SMILES: COc1ccccc1-c1cc(NC(=O)C2(c3ccc4c(c3)OCO4)CC2)ccc1F] | 903 |
| 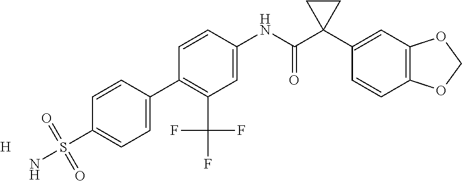 | 904 |
| 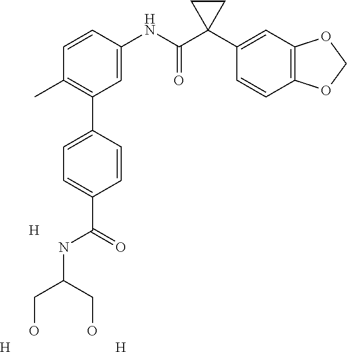 | 905 |
| 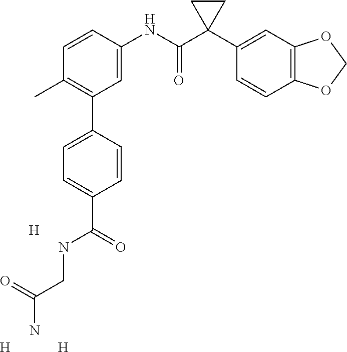 | 906 |
| 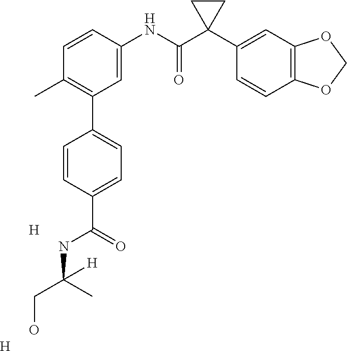 | 907 |
| 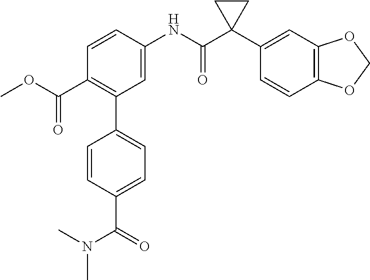 [SMILES: COC(=O)c1ccc(NC(=O)C2(c3ccc4c(c3)OCO4)CC2)cc1-c1ccc(C(=O)N(C)C)cc1] | 908 |
| 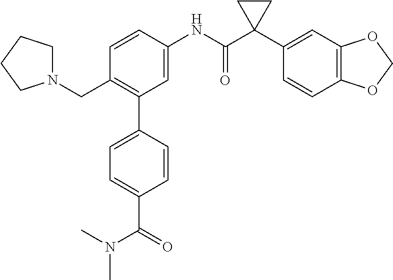 [SMILES: CN(C)C(=O)c1ccc(-c2cc(NC(=O)C3(c4ccc5c(c4)OCO5)CC3)ccc2CN2CCCC2)cc1] | 909 |
| 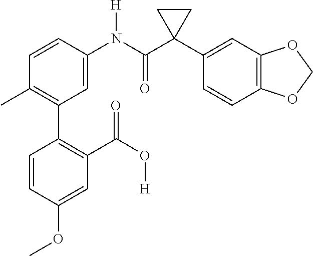 [SMILES: COc1ccc(-c2cc(NC(=O)C3(c4ccc5c(c4)OCO5)CC3)ccc2C)c(C(=O)O)c1] | 910 |
| 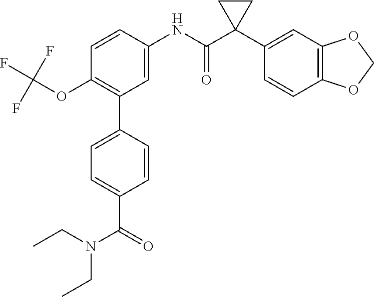 [SMILES: CCN(CC)C(=O)c1ccc(-c2cc(NC(=O)C3(c4ccc5c(c4)OCO5)CC3)ccc2OC(F)(F)F)cc1] | 911 |
| 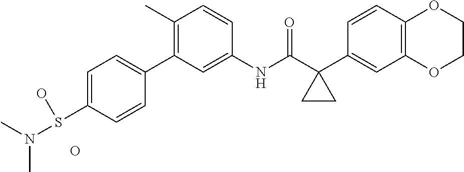 | 912 |
| 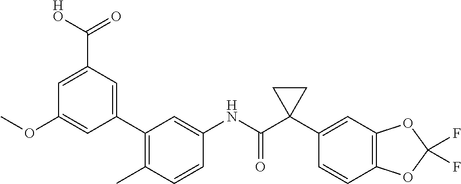 [SMILES: COc1cc(C(=O)O)cc(-c2cc(NC(=O)C3(c4ccc5c(c4)OC(F)(F)O5)CC3)ccc2C)c1] | 913 |
| 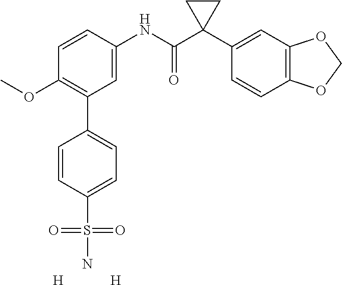 | 914 |
| 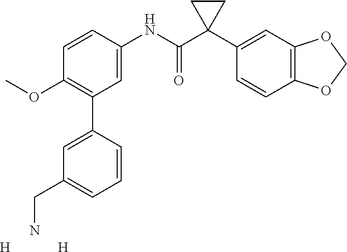 | 915 |
| 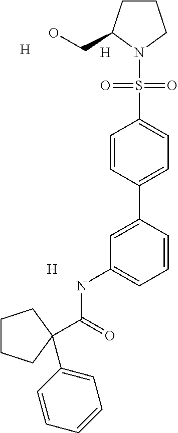 | 916 |
| 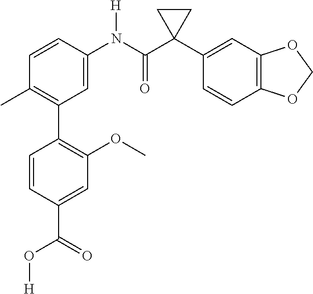 [SMILES: COc1cc(C(=O)O)ccc1-c1cc(NC(=O)C2(c3ccc4c(c3)OCO4)CC2)ccc1C] | 917 |
| 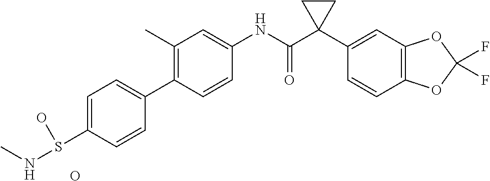 | 918 |
| 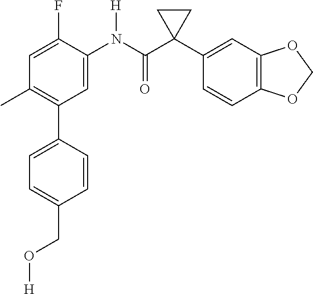 [SMILES: Cc1cc(F)c(NC(=O)C2(c3ccc4c(c3)OCO4)CC2)cc1-c1ccc(CO)cc1] | 919 |
| 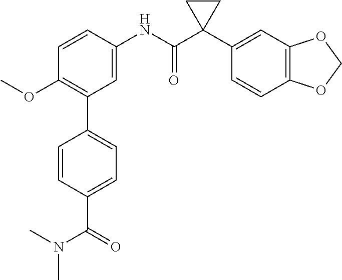 [SMILES: COc1ccc(NC(=O)C2(c3ccc4c(c3)OCO4)CC2)cc1-c1ccc(C(=O)N(C)C)cc1] | 920 |
| 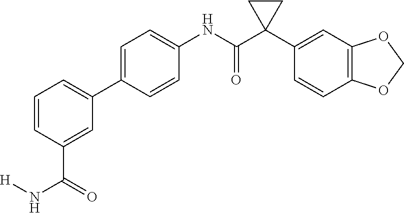 [SMILES: NC(=O)c1cccc(-c2ccc(NC(=O)C3(c4ccc5c(c4)OCO5)CC3)cc2)c1] | 921 |
| 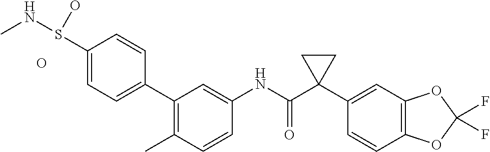 | 922 |
| 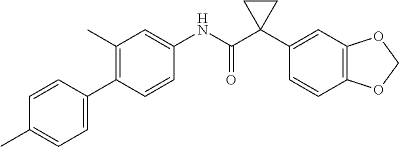 [SMILES: Cc1ccc(-c2ccc(NC(=O)C3(c4ccc5c(c4)OCO5)CC3)cc2C)cc1] | 923 |
| 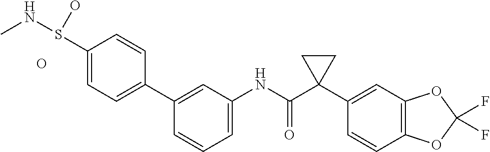 | 924 |
| 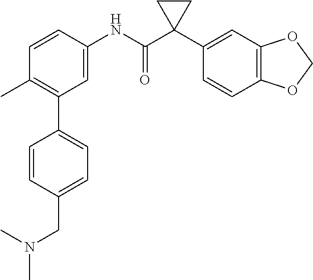 [SMILES: Cc1ccc(NC(=O)C2(c3ccc4c(c3)OCO4)CC2)cc1-c1ccc(CN(C)C)cc1] | 925 |
| 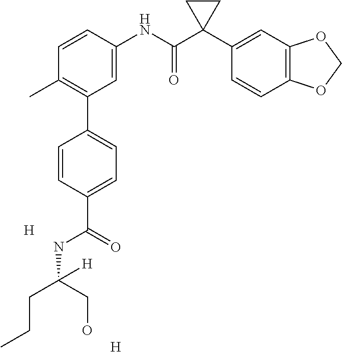 | 926 |
| 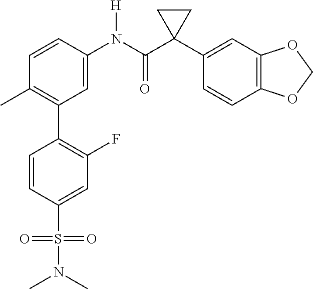 [SMILES: Cc1ccc(NC(=O)C2(c3ccc4c(c3)OCO4)CC2)cc1-c1ccc(S(=O)(=O)N(C)C)cc1F] | 927 |
| 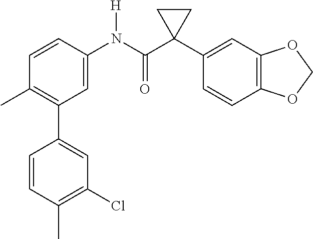 [SMILES: Cc1ccc(-c2cc(NC(=O)C3(c4ccc5c(c4)OCO5)CC3)ccc2C)cc1Cl] | 928 |
| 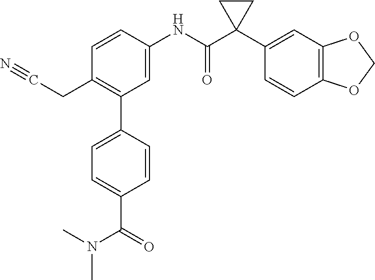 [SMILES: CN(C)C(=O)c1ccc(-c2cc(NC(=O)C3(c4ccc5c(c4)OCO5)CC3)ccc2CC#N)cc1] | 929 |
| 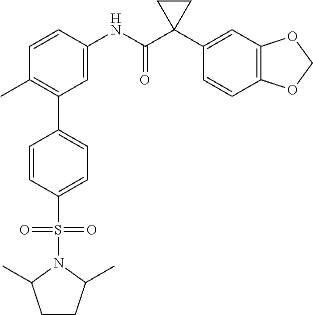 [SMILES: Cc1ccc(NC(=O)C2(c3ccc4c(c3)OCO4)CC2)cc1-c1ccc(S(=O)(=O)N2C(C)CCC2C)cc1] | 930 |
| 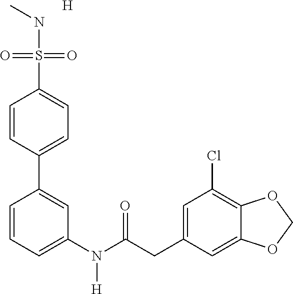 | 931 |
| 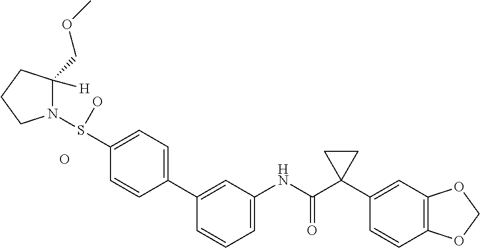 | 932 |
| 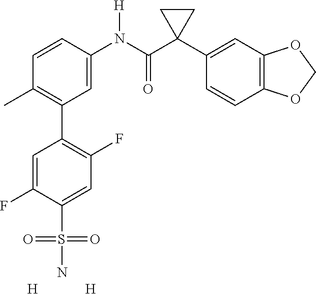 | 933 |
| 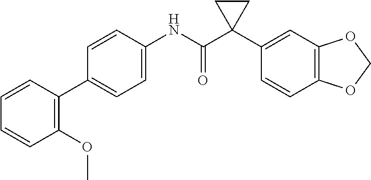 [SMILES: COc1ccccc1-c1ccc(NC(=O)C2(c3ccc4c(c3)OCO4)CC2)cc1] | 934 |
| 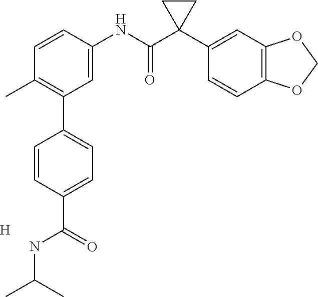 | 935 |
| 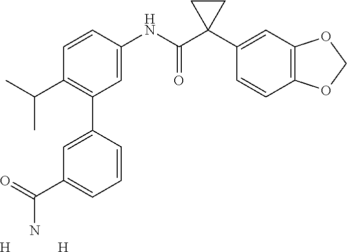 | 936 |
| 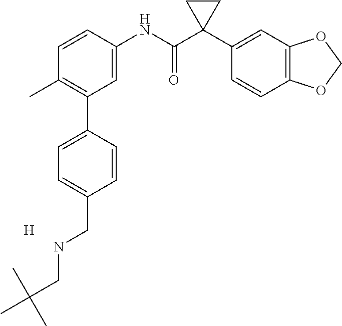 | 937 |
| 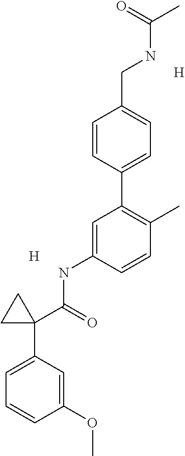 | 938 |
| 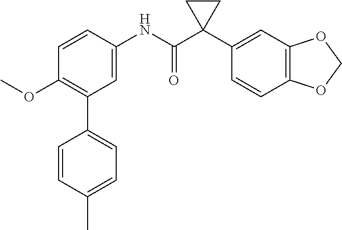 [SMILES: COc1ccc(NC(=O)C2(c3ccc4c(c3)OCO4)CC2)cc1-c1ccc(C)cc1] | 939 |
| 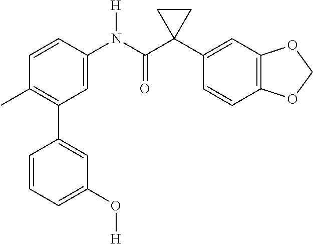 [SMILES: Cc1ccc(NC(=O)C2(c3ccc4c(c3)OCO4)CC2)cc1-c1cccc(O)c1] | 940 |
| 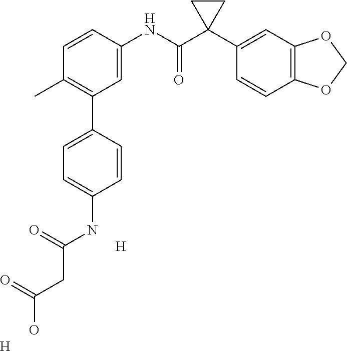 | 941 |
| 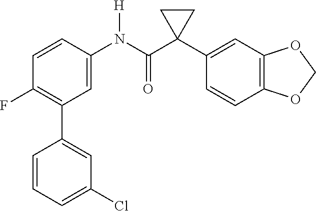 [SMILES: O=C(Nc1ccc(F)c(-c2cccc(Cl)c2)c1)C1(c2ccc3c(c2)OCO3)CC1] | 942 |
| 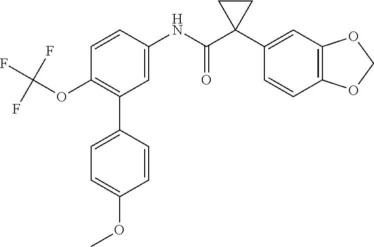 [SMILES: COc1ccc(-c2cc(NC(=O)C3(c4ccc5c(c4)OCO5)CC3)ccc2OC(F)(F)F)cc1] | 943 |
| 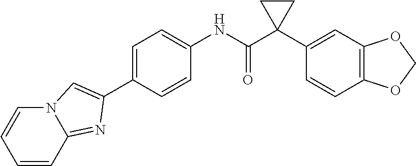 [SMILES: O=C(Nc1ccc(-c2cn3ccccc3n2)cc1)C1(c2ccc3c(c2)OCO3)CC1] | 944 |
| 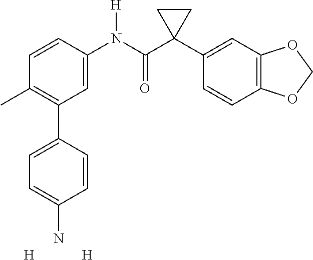 | 945 |
| 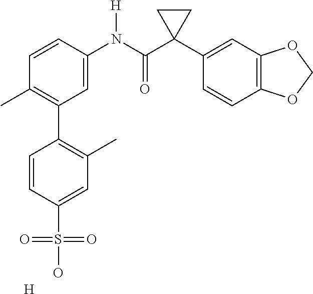 | 946 |
| 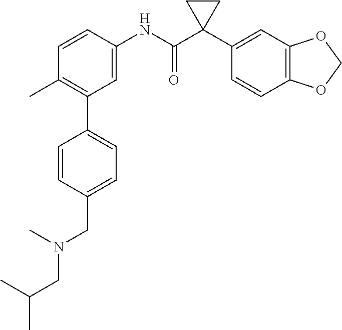 [SMILES: Cc1ccc(NC(=O)C2(c3ccc4c(c3)OCO4)CC2)cc1-c1ccc(CN(C)CC(C)C)cc1] | 947 |
| 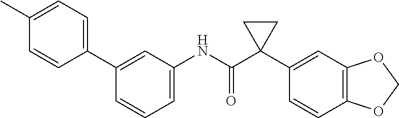 [SMILES: Cc1ccc(-c2cccc(NC(=O)C3(c4ccc5c(c4)OCO5)CC3)c2)cc1] | 948 |
| 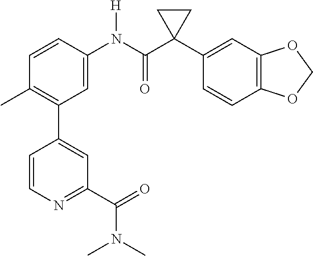 [SMILES: Cc1ccc(NC(=O)C2(c3ccc4c(c3)OCO4)CC2)cc1-c1ccnc(C(=O)N(C)C)c1] | 949 |
| 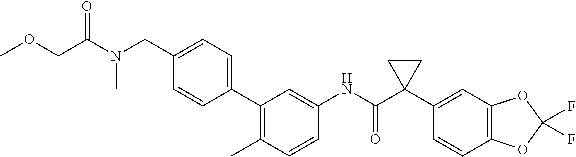 [SMILES: COCC(=O)N(C)Cc1ccc(-c2cc(NC(=O)C3(c4ccc5c(c4)OC(F)(F)O5)CC3)ccc2C)cc1] | 950 |
| 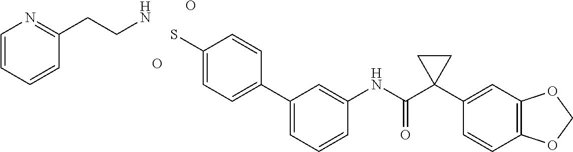 | 951 |
| 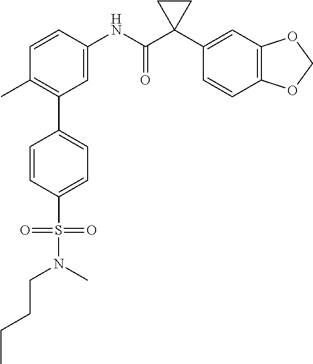 [SMILES: CCCCN(C)S(=O)(=O)c1ccc(-c2cc(NC(=O)C3(c4ccc5c(c4)OCO5)CC3)ccc2C)cc1] | 952 |
| 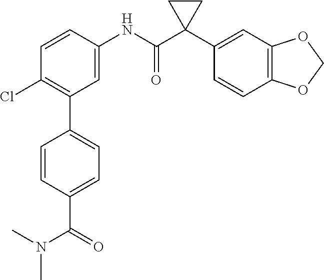 [SMILES: CN(C)C(=O)c1ccc(-c2cc(NC(=O)C3(c4ccc5c(c4)OCO5)CC3)ccc2Cl)cc1] | 953 |
| 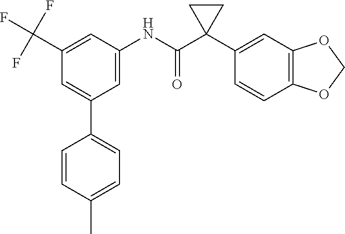 [SMILES: Cc1ccc(-c2cc(NC(=O)C3(c4ccc5c(c4)OCO5)CC3)cc(C(F)(F)F)c2)cc1] | 954 |
| 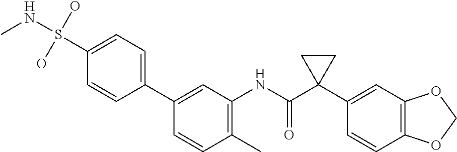 [SMILES: CNS(O)(O)c1ccc(-c2ccc(C)c(NC(=O)C3(c4ccc5c(c4)OCO5)CC3)c2)cc1] | 955 |
| 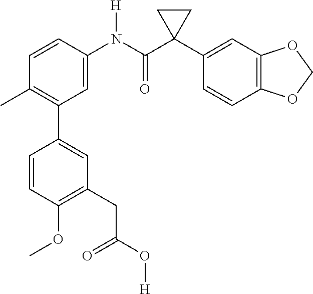 [SMILES: COc1ccc(-c2cc(NC(=O)C3(c4ccc5c(c4)OCO5)CC3)ccc2C)cc1CC(=O)O] | 956 |
| 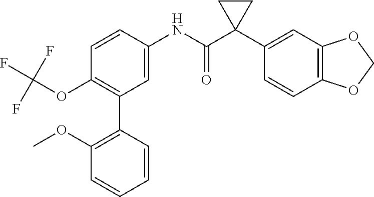 [SMILES: COc1ccccc1-c1cc(NC(=O)C2(c3ccc4c(c3)OCO4)CC2)ccc1OC(F)(F)F] | 957 |
| 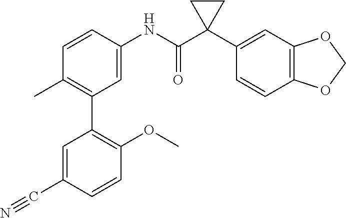 | 958 |
| 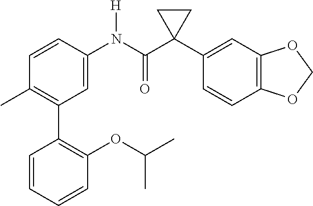 | 960 |
| 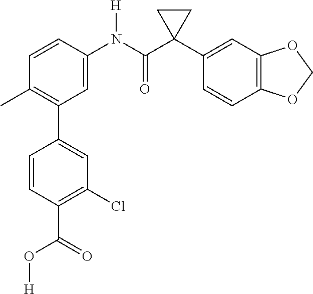 [SMILES: Cc1ccc(NC(=O)C2(c3ccc4c(c3)OCO4)CC2)cc1-c1ccc(C(=O)O)c(Cl)c1] | 961 |
| 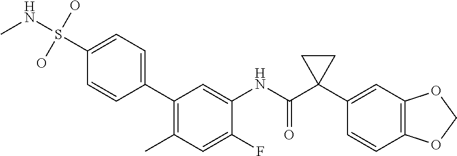 [SMILES: CNS(O)(O)c1ccc(-c2cc(NC(=O)C3(c4ccc5c(c4)OCO5)CC3)c(F)cc2C)cc1] | 962 |
| 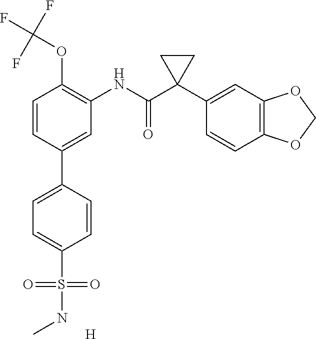 | 963 |
| 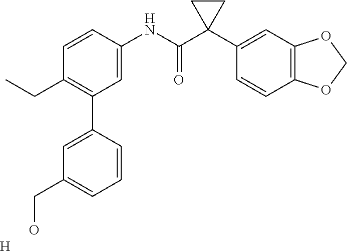 | 964 |
| 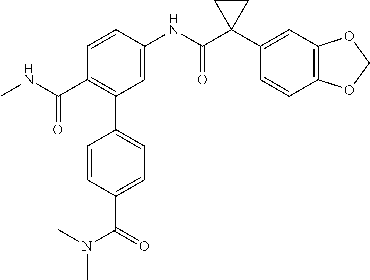 [SMILES: CNC(=O)c1ccc(NC(=O)C2(c3ccc4c(c3)OCO4)CC2)cc1-c1ccc(C(=O)N(C)C)cc1] | 965 |
| 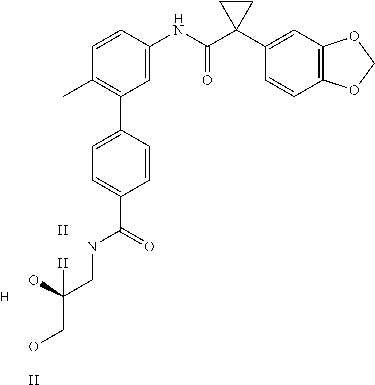 | 966 |
| 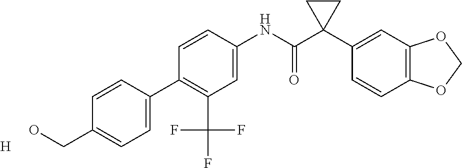 | 967 |
| 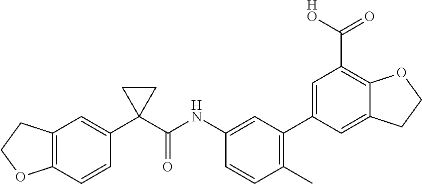 [SMILES: Cc1ccc(NC(=O)C2(c3ccc4c(c3)CCO4)CC2)cc1-c1cc2c(c(C(=O)O)c1)OCC2] | 968 |
| 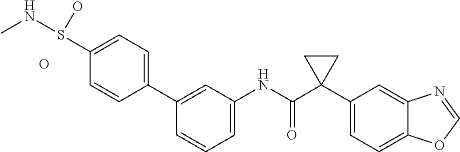 | 969 |
| 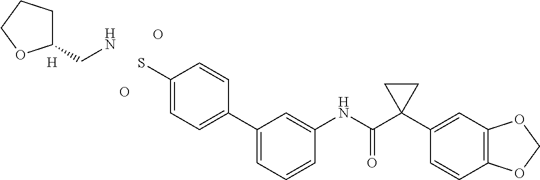 | 970 |
| 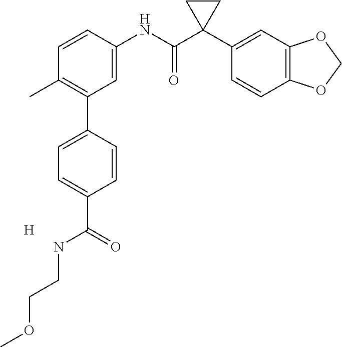 | 971 |
| 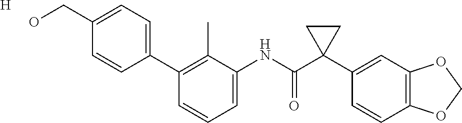 | 972 |
| 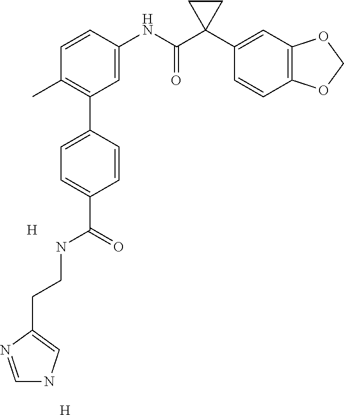 | 973 |
| 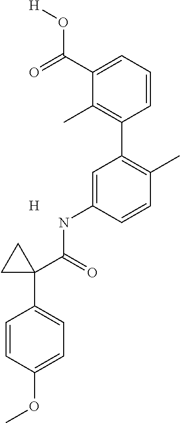 | 974 |
| 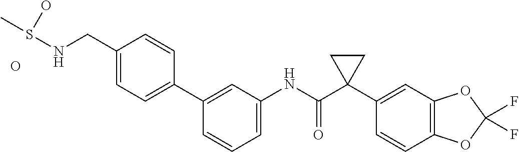 | 975 |
| 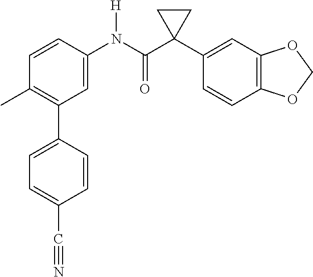 [SMILES: Cc1ccc(NC(=O)C2(c3ccc4c(c3)OCO4)CC2)cc1-c1ccc(C#N)cc1] | 976 |
| 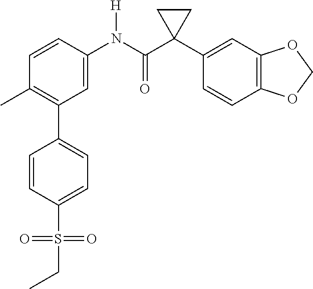 [SMILES: CCS(=O)(=O)c1ccc(-c2cc(NC(=O)C3(c4ccc5c(c4)OCO5)CC3)ccc2C)cc1] | 977 |
| 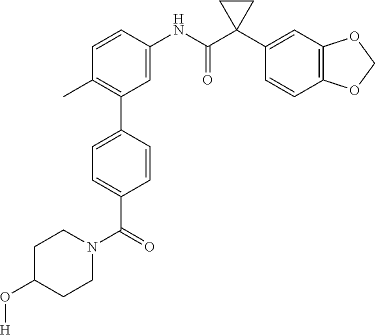 [SMILES: Cc1ccc(NC(=O)C2(c3ccc4c(c3)OCO4)CC2)cc1-c1ccc(C(=O)N2CCC(O)CC2)cc1] | 978 |
| 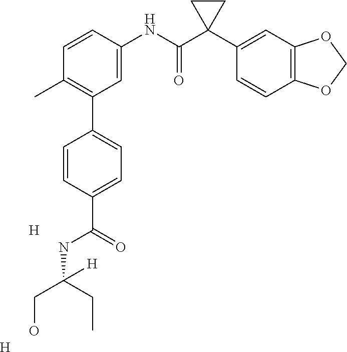 | 979 |
| 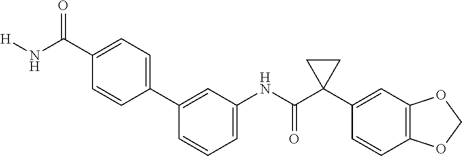 [SMILES: NC(=O)c1ccc(-c2cccc(NC(=O)C3(c4ccc5c(c4)OCO5)CC3)c2)cc1] | 980 |
| 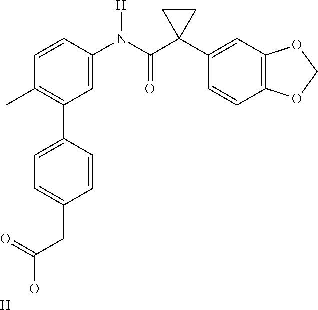 | 981 |
| 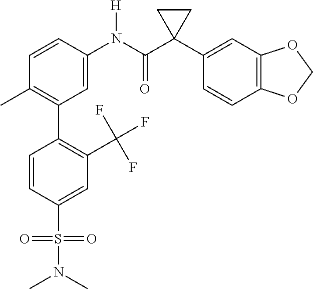 [SMILES: Cc1ccc(NC(=O)C2(c3ccc4c(c3)OCO4)CC2)cc1-c1ccc(S(=O)(=O)N(C)C)cc1C(F)(F)F] | 982 |
| 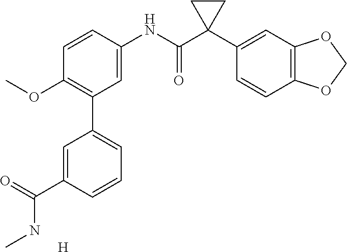 | 983 |
| 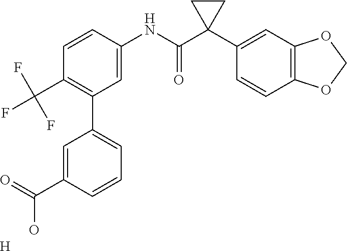 | 984 |
| 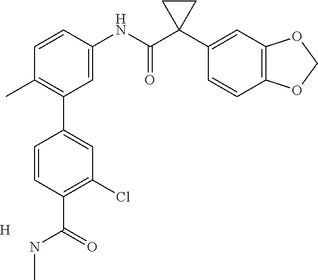 | 985 |
| 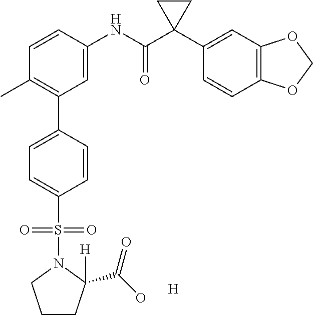 | 986 |
| 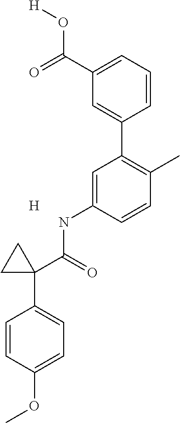 | 987 |
| 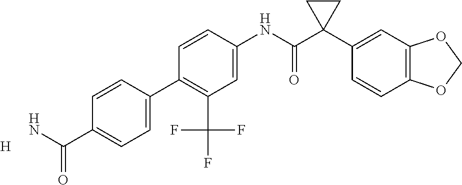 | 988 |
| 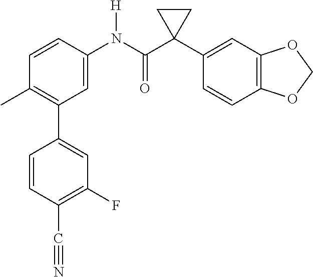 [SMILES: Cc1ccc(NC(=O)C2(c3ccc4c(c3)OCO4)CC2)cc1-c1ccc(C#N)c(F)c1] | 989 |
| 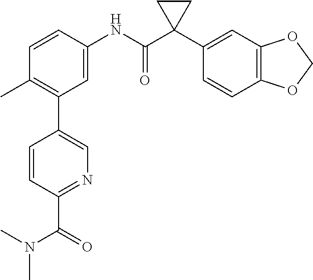 [SMILES: Cc1ccc(NC(=O)C2(c3ccc4c(c3)OCO4)CC2)cc1-c1ccc(C(=O)N(C)C)nc1] | 990 |
| 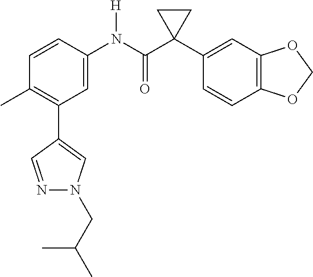 [SMILES: Cc1ccc(NC(=O)C2(c3ccc4c(c3)OCO4)CC2)cc1-c1cnn(CC(C)C)c1] | 991 |
| 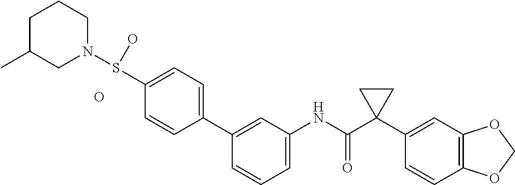 | 992 |
| 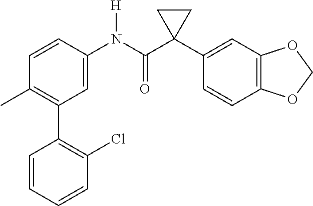 [SMILES: Cc1ccc(NC(=O)C2(c3ccc4c(c3)OCO4)CC2)cc1-c1ccccc1Cl] | 993 |
| 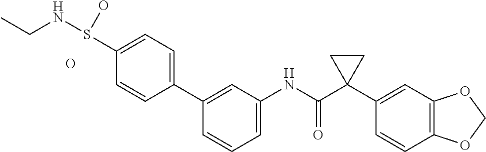 | 994 |
| 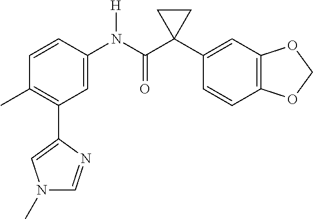 [SMILES: Cc1ccc(NC(=O)C2(c3ccc4c(c3)OCO4)CC2)cc1-c1cn(C)cn1] | 995 |
| 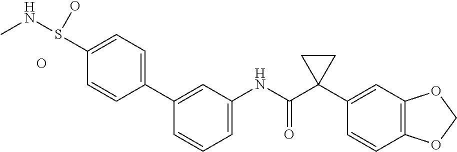 | 996 |
| 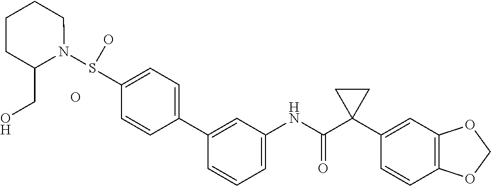 | 997 |
| 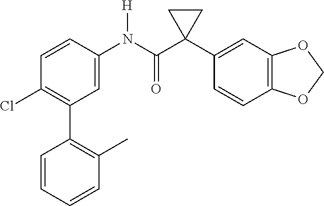 [SMILES: Cc1ccccc1-c1cc(NC(=O)C2(c3ccc4c(c3)OCO4)CC2)ccc1Cl] | 998 |
| 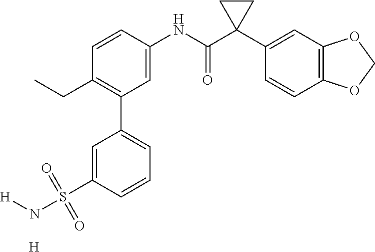 | 999 |
| 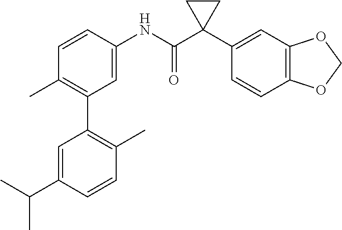 [SMILES: Cc1ccc(NC(=O)C2(c3ccc4c(c3)OCO4)CC2)cc1-c1cc(C(C)C)ccc1C] | 1000 |
| 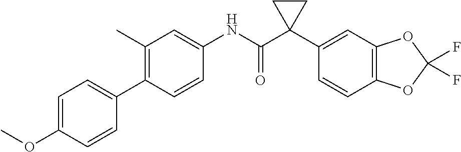 [SMILES: COc1ccc(-c2ccc(NC(=O)C3(c4ccc5c(c4)OC(F)(F)O5)CC3)cc2C)cc1] | 1001 |
| 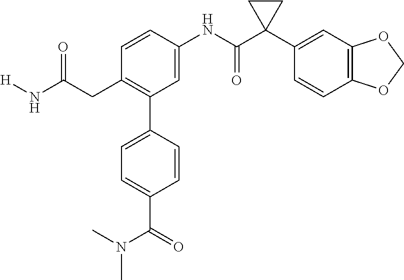 [SMILES: CN(C)C(=O)c1ccc(-c2cc(NC(=O)C3(c4ccc5c(c4)OCO5)CC3)ccc2CC(N)=O)cc1] | 1002 |
| 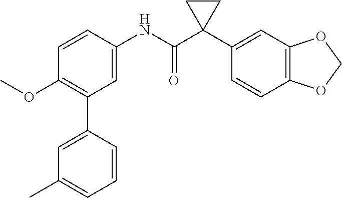 [SMILES: COc1ccc(NC(=O)C2(c3ccc4c(c3)OCO4)CC2)cc1-c1cccc(C)c1] | 1003 |
| 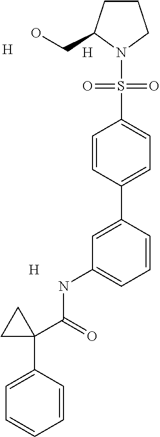 | 1004 |
| 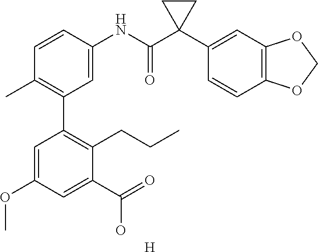 | 1005 |
| 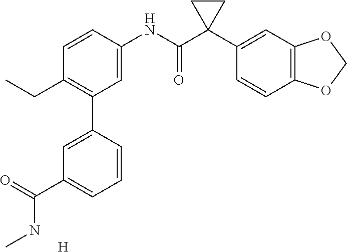 | 1006 |
| 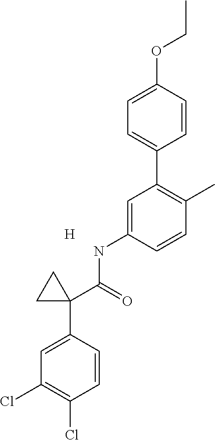 | 1007 |
| 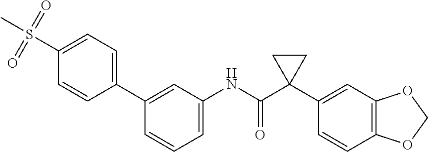 [SMILES: CS(=O)(=O)c1ccc(-c2cccc(NC(=O)C3(c4ccc5c(c4)OCO5)CC3)c2)cc1] | 1008 |
| 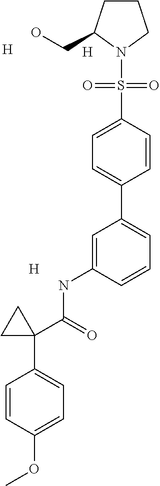 | 1009 |
| 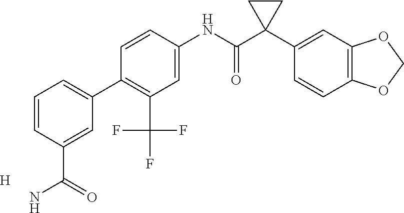 | 1010 |
| 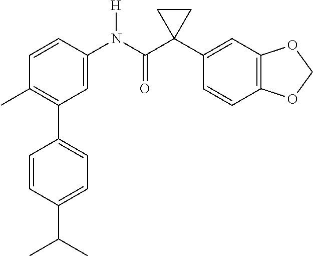 [SMILES: Cc1ccc(NC(=O)C2(c3ccc4c(c3)OCO4)CC2)cc1-c1ccc(C(C)C)cc1] | 1011 |
| 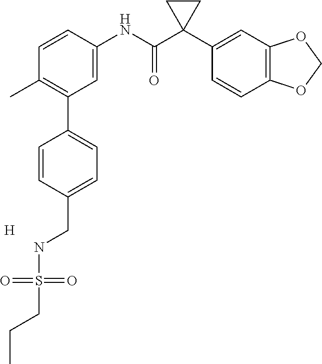 | 1012 |
| 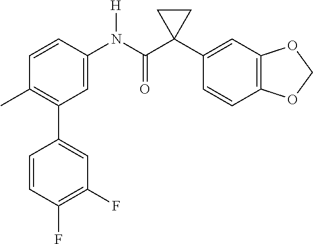 [SMILES: Cc1ccc(NC(=O)C2(c3ccc4c(c3)OCO4)CC2)cc1-c1ccc(F)c(F)c1] | 1013 |
| 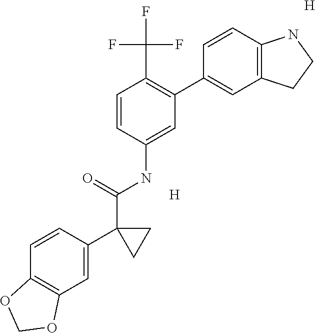 | 1014 |
| 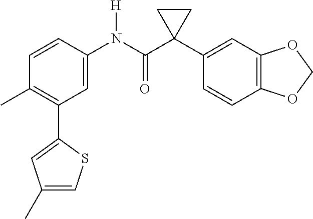 [SMILES: Cc1csc(-c2cc(NC(=O)C3(c4ccc5c(c4)OCO5)CC3)ccc2C)c1] | 1015 |
| 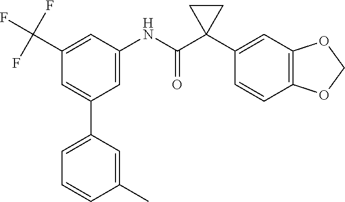 [SMILES: Cc1cccc(-c2cc(NC(=O)C3(c4ccc5c(c4)OCO5)CC3)cc(C(F)(F)F)c2)c1] | 1016 |
| 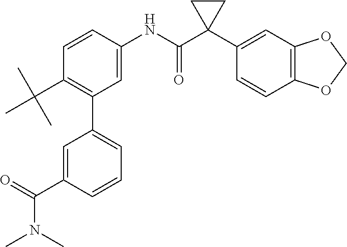 [SMILES: CN(C)C(=O)c1cccc(-c2cc(NC(=O)C3(c4ccc5c(c4)OCO5)CC3)ccc2C(C)(C)C)c1] | 1017 |
| 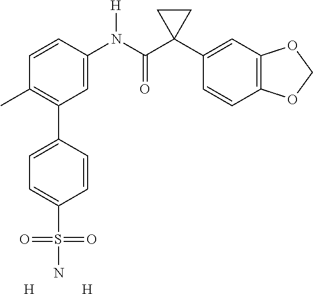 | 1018 |
| 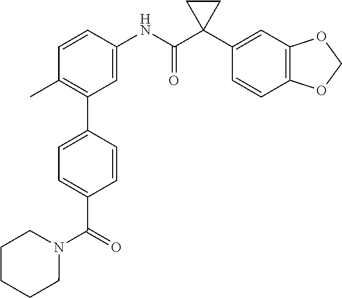 [SMILES: Cc1ccc(NC(=O)C2(c3ccc4c(c3)OCO4)CC2)cc1-c1ccc(C(=O)N2CCCCC2)cc1] | 1019 |
| 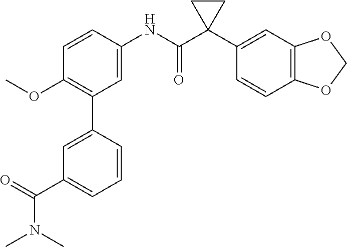 [SMILES: COc1ccc(NC(=O)C2(c3ccc4c(c3)OCO4)CC2)cc1-c1cccc(C(=O)N(C)C)c1] | 1020 |
| 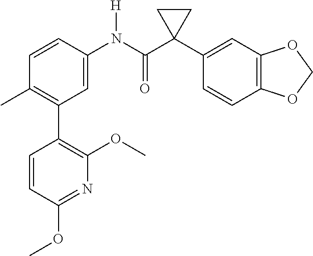 [SMILES: COc1ccc(-c2cc(NC(=O)C3(c4ccc5c(c4)OCO5)CC3)ccc2C)c(OC)n1] | 1021 |
| 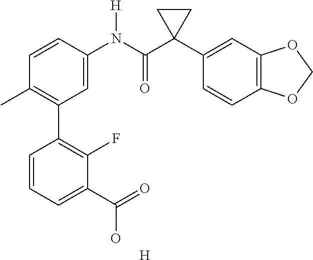 | 1022 |
| 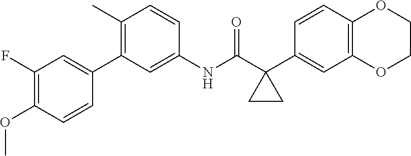 | 1023 |
| 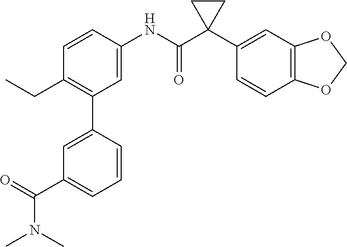 [SMILES: CCc1ccc(NC(=O)C2(c3ccc4c(c3)OCO4)CC2)cc1-c1cccc(C(=O)N(C)C)c1] | 1024 |
| 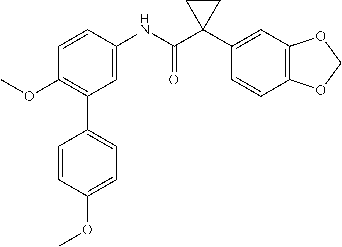 [SMILES: COc1ccc(-c2cc(NC(=O)C3(c4ccc5c(c4)OCO5)CC3)ccc2OC)cc1] | 1025 |
| 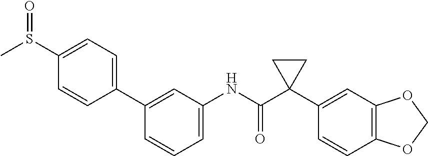 [SMILES: CS(=O)c1ccc(-c2cccc(NC(=O)C3(c4ccc5c(c4)OCO5)CC3)c2)cc1] | 1026 |
| 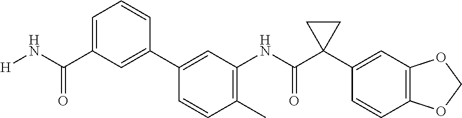 [SMILES: Cc1ccc(-c2cccc(C(N)=O)c2)cc1NC(=O)C1(c2ccc3c(c2)OCO3)CC1] | 1027 |
| 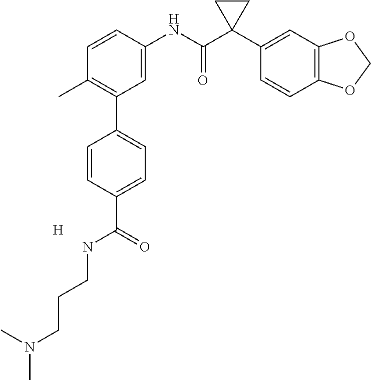 | 1028 |
| 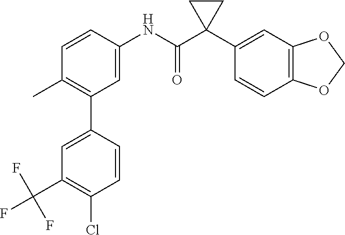 [SMILES: Cc1ccc(NC(=O)C2(c3ccc4c(c3)OCO4)CC2)cc1-c1ccc(Cl)c(C(F)(F)F)c1] | 1029 |
| 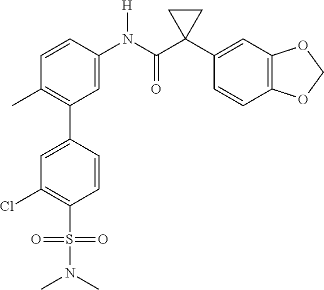 [SMILES: Cc1ccc(NC(=O)C2(c3ccc4c(c3)OCO4)CC2)cc1-c1ccc(S(=O)(=O)N(C)C)c(Cl)c1] | 1030 |
| 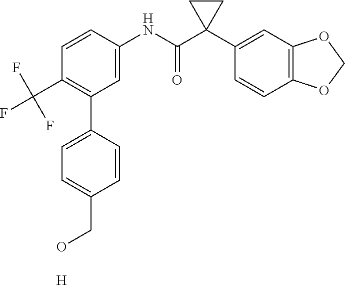 | 1031 |
| 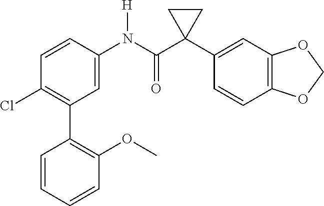 [SMILES: COc1ccccc1-c1cc(NC(=O)C2(c3ccc4c(c3)OCO4)CC2)ccc1Cl] | 1032 |
| 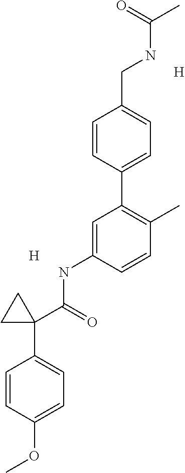 | 1033 |
| 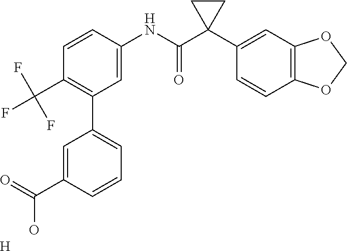 | 1034 |
| 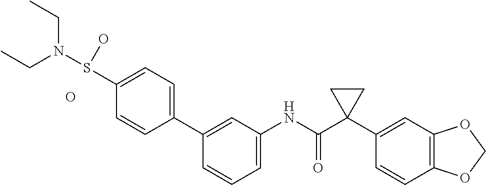 | 1035 |
| 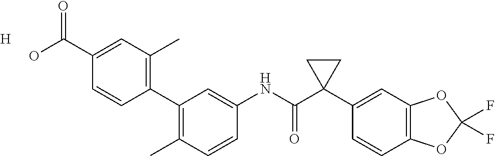 | 1036 |
| 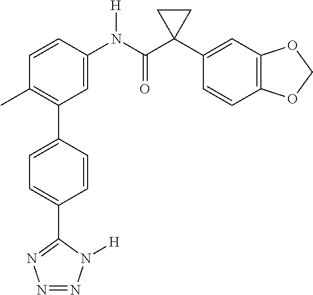 [SMILES: Cc1ccc(NC(=O)C2(c3ccc4c(c3)OCO4)CC2)cc1-c1ccc(-c2nnn[nH]2)cc1] | 1037 |
| 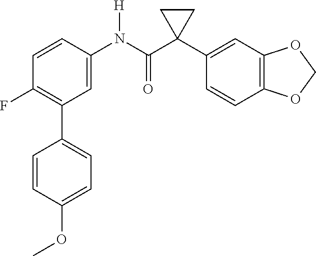 [SMILES: COc1ccc(-c2cc(NC(=O)C3(c4ccc5c(c4)OCO5)CC3)ccc2F)cc1] | 1038 |
| 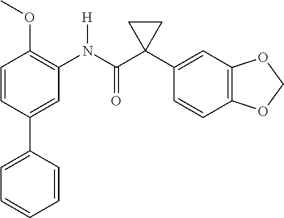 [SMILES: COc1ccc(-c2ccccc2)cc1NC(=O)C1(c2ccc3c(c2)OCO3)CC1] | 1039 |
| 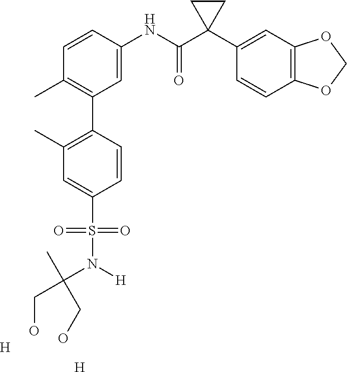 | 1040 |
| 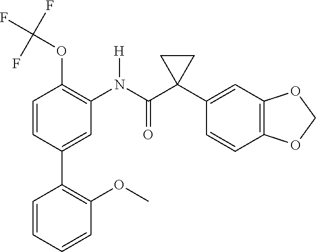 [SMILES: COc1ccccc1-c1ccc(OC(F)(F)F)c(NC(=O)C2(c3ccc4c(c3)OCO4)CC2)c1] | 1041 |
| 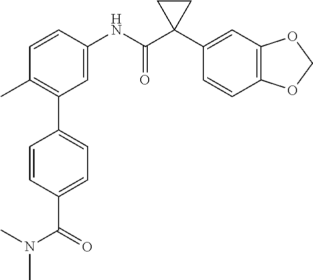 [SMILES: Cc1ccc(NC(=O)C2(c3ccc4c(c3)OCO4)CC2)cc1-c1ccc(C(=O)N(C)C)cc1] | 1042 |
| 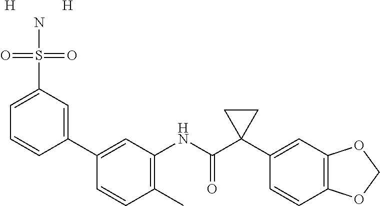 | 1043 |
| 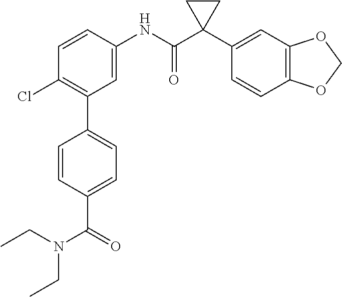 [SMILES: CCN(CC)C(=O)c1ccc(-c2cc(NC(=O)C3(c4ccc5c(c4)OCO5)CC3)ccc2Cl)cc1] | 1044 |
| 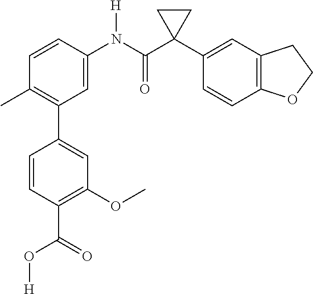 [SMILES: COc1cc(-c2cc(NC(=O)C3(c4ccc5c(c4)CCO5)CC3)ccc2C)ccc1C(=O)O] | 1045 |
| 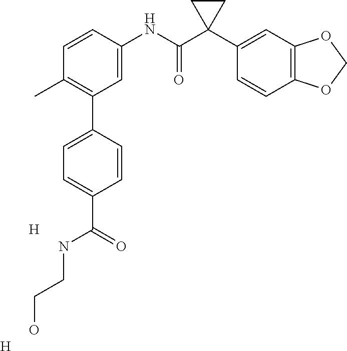 | 1046 |
| 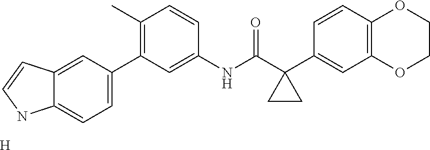 | 1047 |
| 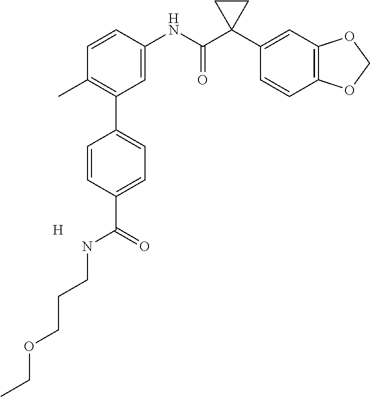 | 1048 |
| 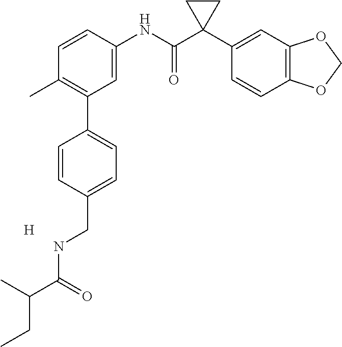 | 1049 |
| 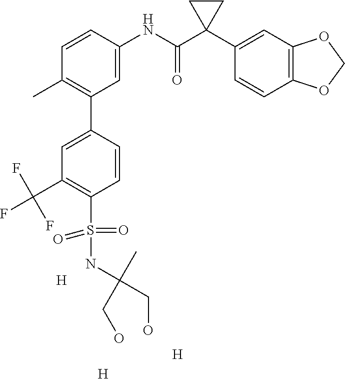 | 1050 |
| 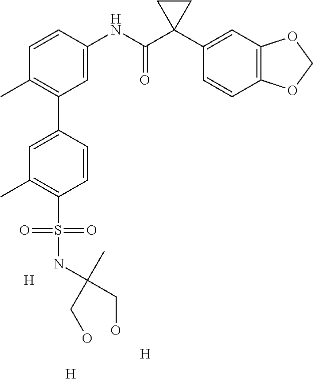 | 1051 |
| 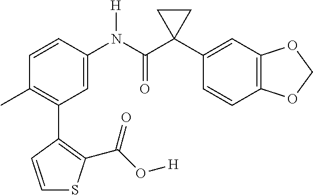 [SMILES: Cc1ccc(NC(=O)C2(c3ccc4c(c3)OCO4)CC2)cc1-c1ccsc1C(=O)O] | 1052 |
| 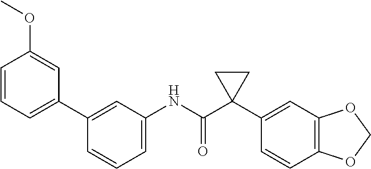 [SMILES: COc1cccc(-c2cccc(NC(=O)C3(c4ccc5c(c4)OCO5)CC3)c2)c1] | 1053 |
| 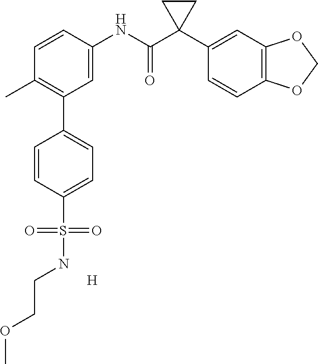 | 1054 |
| 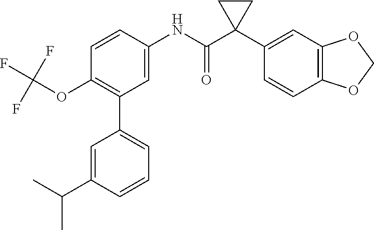 [SMILES: CC(C)c1cccc(-c2cc(NC(=O)C3(c4ccc5c(c4)OCO5)CC3)ccc2OC(F)(F)F)c1] | 1055 |
| 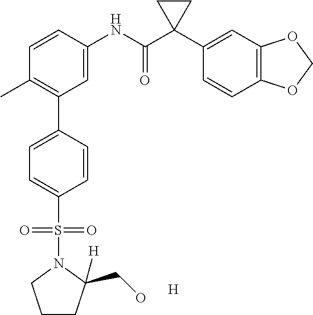 | 1056 |
| 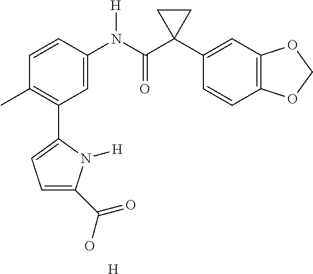 | 1057 |
| 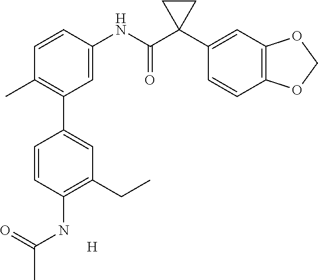 | 1058 |
| 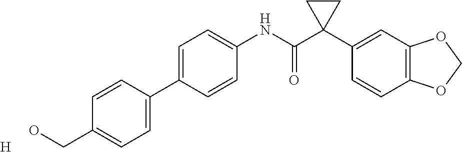 | 1059 |
| 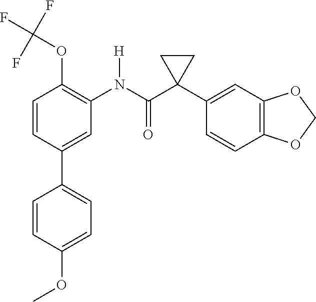 [SMILES: COc1ccc(-c2ccc(OC(F)(F)F)c(NC(=O)C3(c4ccc5c(c4)OCO5)CC3)c2)cc1] | 1060 |
| 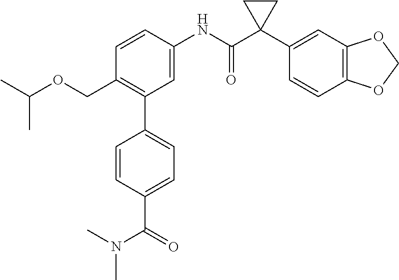 [SMILES: CC(C)OCc1ccc(NC(=O)C2(c3ccc4c(c3)OCO4)CC2)cc1-c1ccc(C(=O)N(C)C)cc1] | 1061 |
| 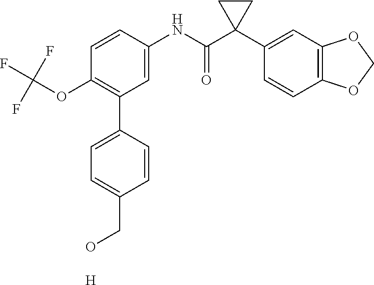 | 1062 |
| 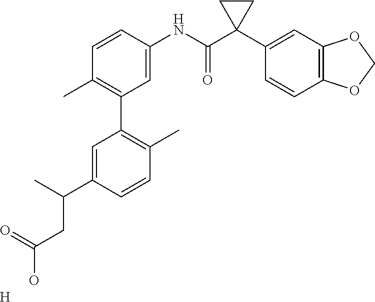 | 1063 |
| 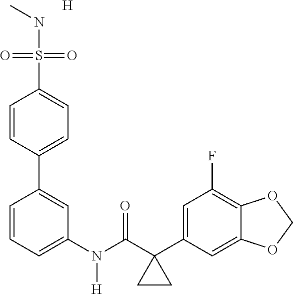 | 1064 |
| 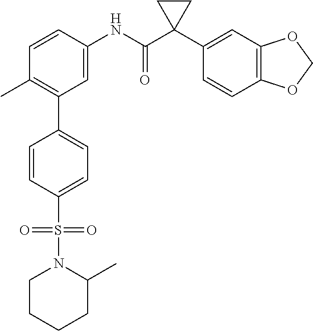 [SMILES: Cc1ccc(NC(=O)C2(c3ccc4c(c3)OCO4)CC2)cc1-c1ccc(S(=O)(=O)N2CCCCC2C)cc1] | 1065 |
| 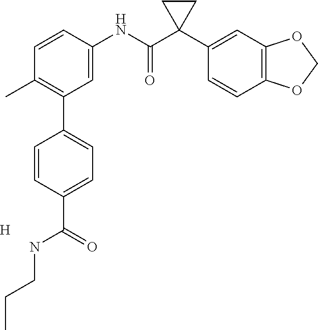 | 1066 |
| 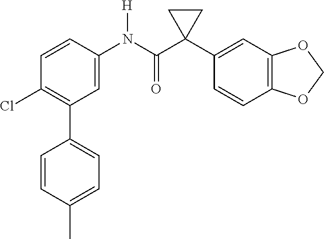 [SMILES: Cc1ccc(-c2cc(NC(=O)C3(c4ccc5c(c4)OCO5)CC3)ccc2Cl)cc1] | 1067 |
| 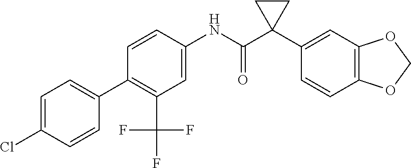 [SMILES: O=C(Nc1ccc(-c2ccc(Cl)cc2)c(C(F)(F)F)c1)C1(c2ccc3c(c2)OCO3)CC1] | 1068 |
| 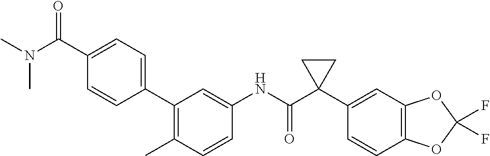 [SMILES: Cc1ccc(NC(=O)C2(c3ccc4c(c3)OC(F)(F)O4)CC2)cc1-c1ccc(C(=O)N(C)C)cc1] | 1069 |
| 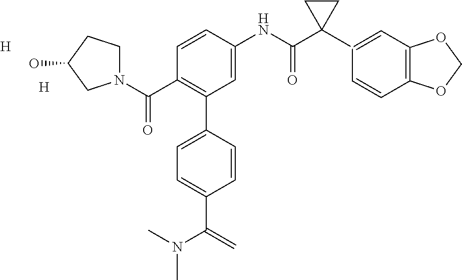 | 1070 |
| 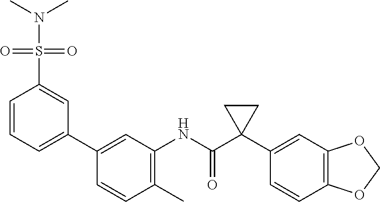 | 1071 |
| 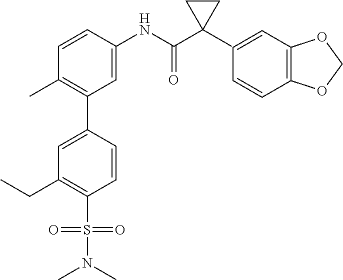 | 1072 |
| 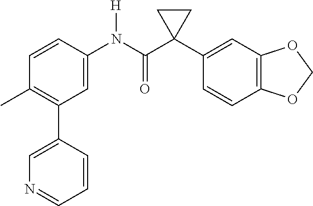 | 1073 |
| 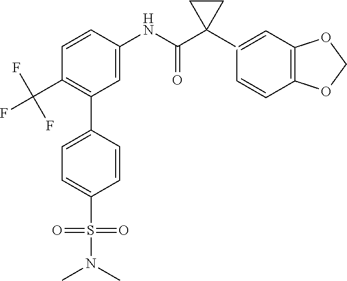 | 1074 |
| 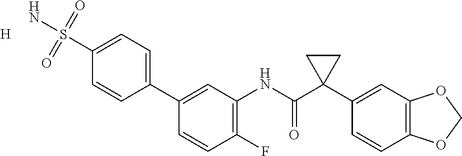 | 1075 |
| 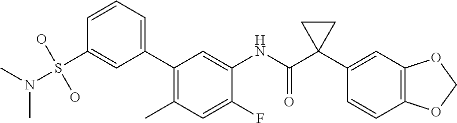 [SMILES: Cc1cc(F)c(NC(=O)C2(c3ccc4c(c3)OCO4)CC2)cc1-c1cccc(S(O)(O)N(C)C)c1] | 1076 |
| 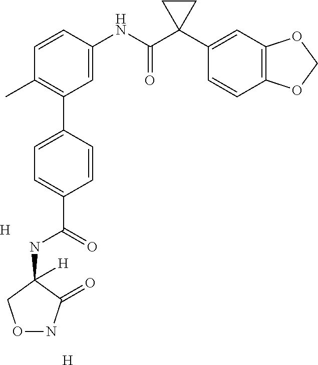 | 1077 |
| 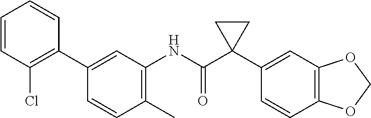 [SMILES: Cc1ccc(-c2ccccc2Cl)cc1NC(=O)C1(c2ccc3c(c2)OCO3)CC1] | 1078 |
| 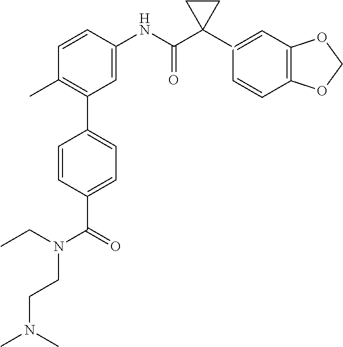 | 1079 |
| 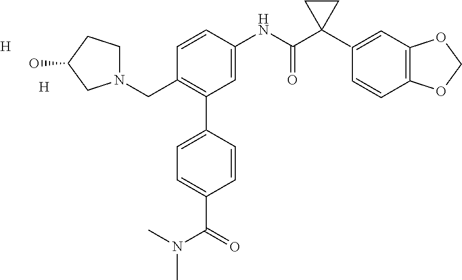 | 1080 |
| 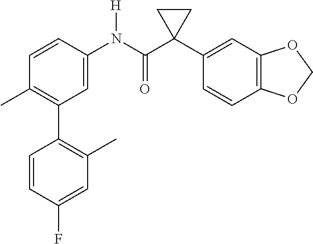 | 1081 |
| 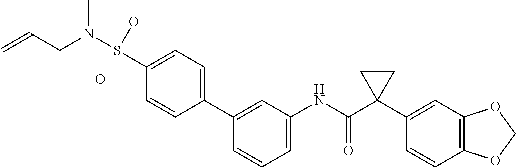 | 1082 |
| 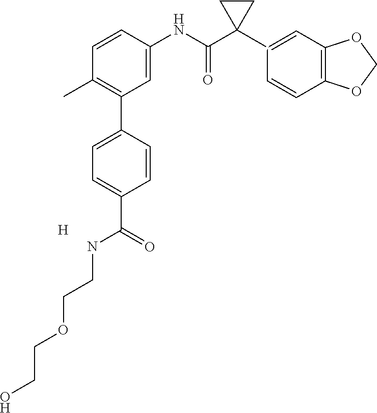 | 1083 |
| 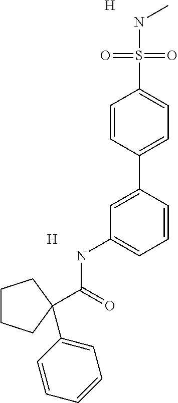 | 1084 |
| 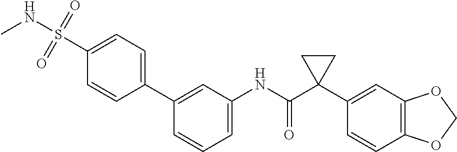 [SMILES: CNS(=O)(=O)c1ccc(-c2cccc(NC(=O)C3(c4ccc5c(c4)OCO5)CC3)c2)cc1] | 1085 |
| 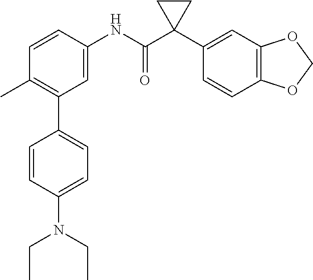 | 1086 |
| 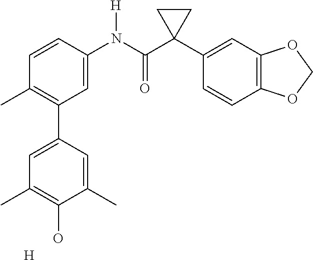 | 1087 |
| 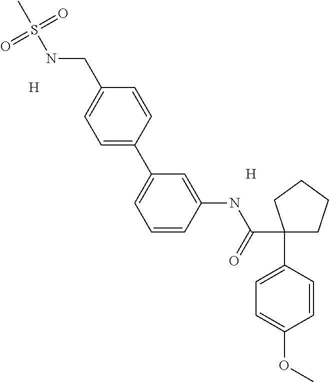 | 1088 |
| 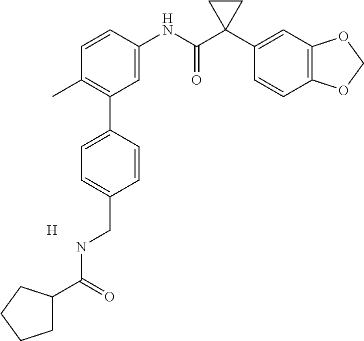 | 1089 |
| 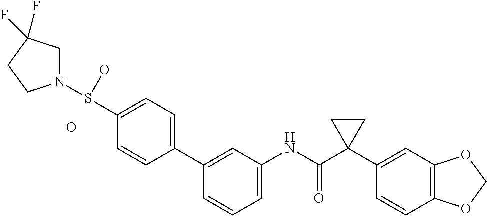 | 1090 |
| 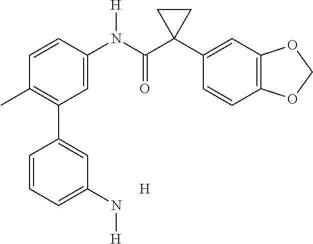 | 1091 |
| 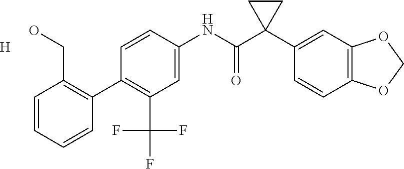 | 1092 |
| 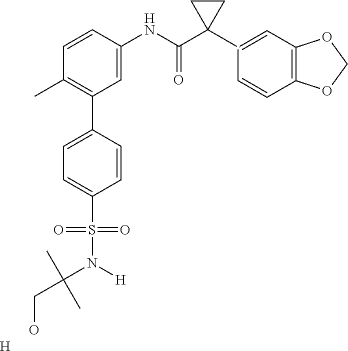 | 1093 |
| 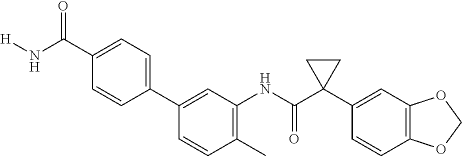 [SMILES: Cc1ccc(-c2ccc(C(N)=O)cc2)cc1NC(=O)C1(c2ccc3c(c2)OCO3)CC1] | 1094 |
| 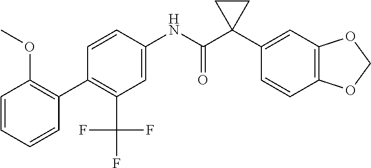 | 1095 |
| 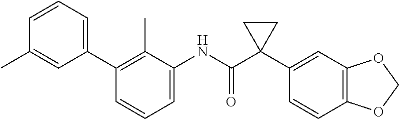 [SMILES: Cc1cccc(-c2cccc(NC(=O)C3(c4ccc5c(c4)OCO5)CC3)c2C)c1] | 1096 |
| 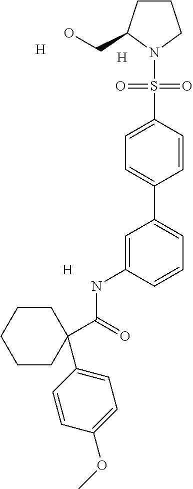 | 1097 |
| 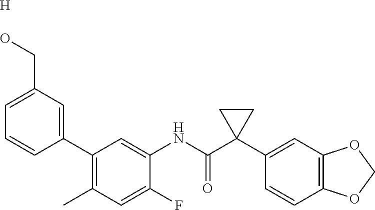 | 1098 |
| 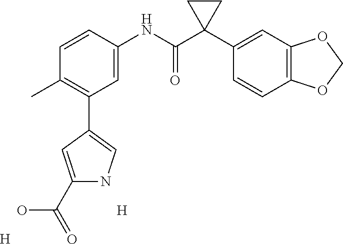 | 1099 |
| 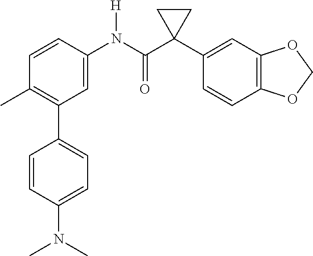 | 1100 |
| 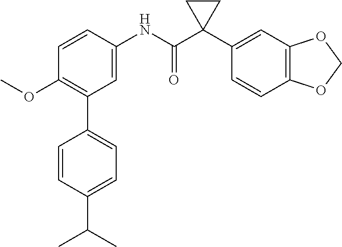 | 1101 |
| 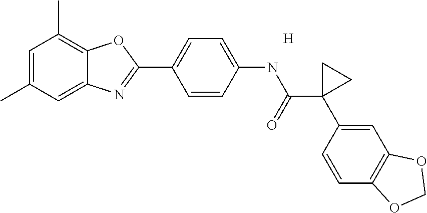 | 1102 |
| 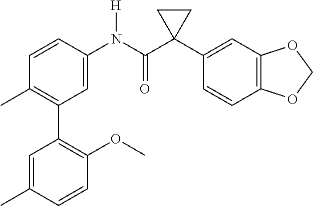 | 1103 |
| 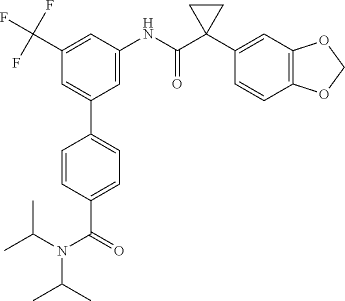 | 1104 |
| 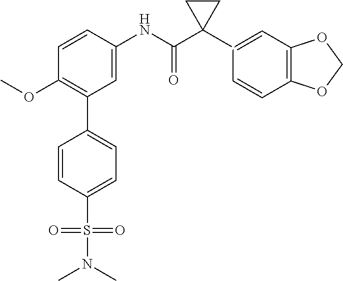 | 1105 |
| 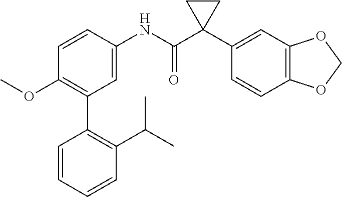 | 1106 |
| 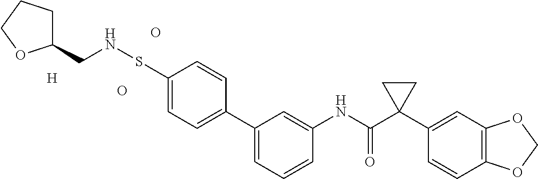 | 1107 |
| 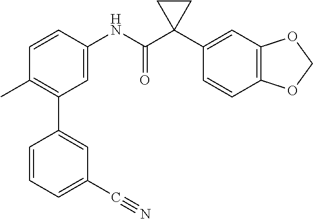 | 1108 |
| 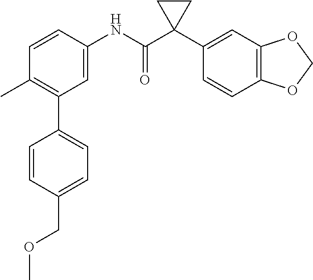 | 1109 |
| 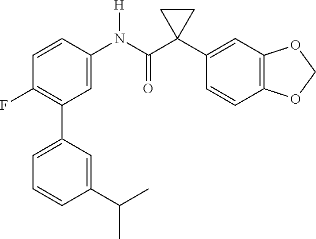 | 1110 |
| 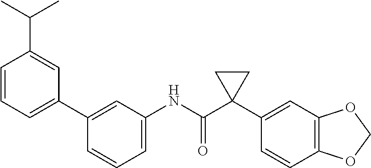 | 1111 |
| 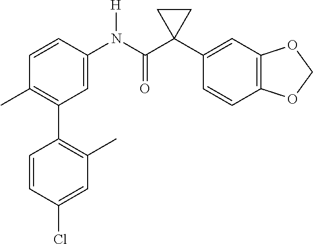 | 1112 |
| 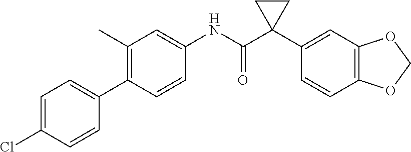 [SMILES: Cc1cc(NC(=O)C2(c3ccc4c(c3)OCO4)CC2)ccc1-c1ccc(Cl)cc1] | 1113 |
| 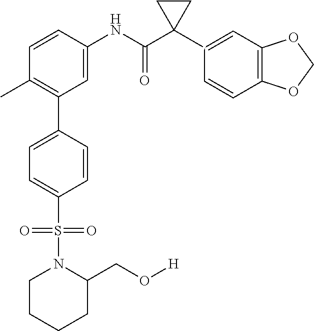 | 1114 |
| 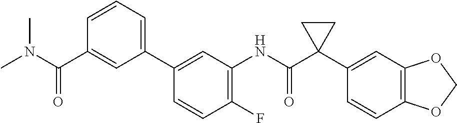 [SMILES: CN(C)C(=O)c1cccc(-c2ccc(F)c(NC(=O)C3(c4ccc5c(c4)OCO5)CC3)c2)c1] | 1115 |
| 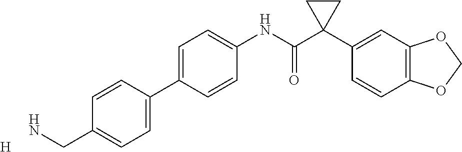 | 1116 |
| 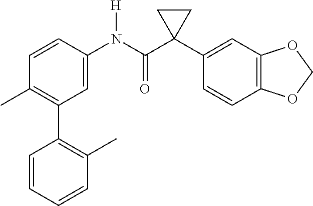 | 1117 |
| 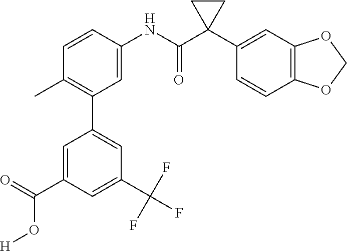 | 1118 |
| 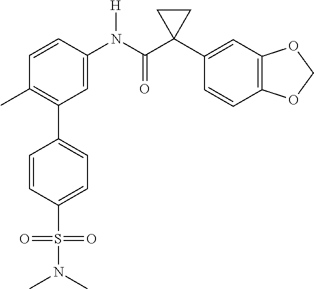 | 1119 |
| 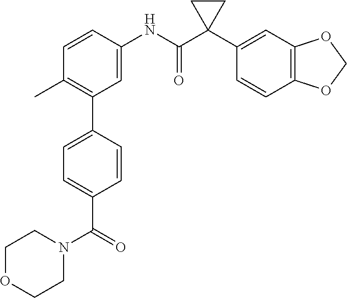 | 1120 |
| 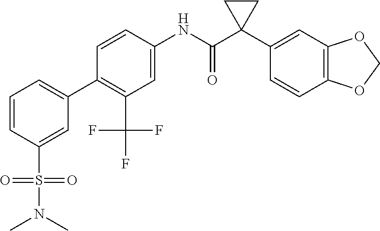 [SMILES: CN(C)S(=O)(=O)c1cccc(-c2ccc(NC(=O)C3(c4ccc5c(c4)OCO5)CC3)cc2C(F)(F)F)c1] | 1121 |
| 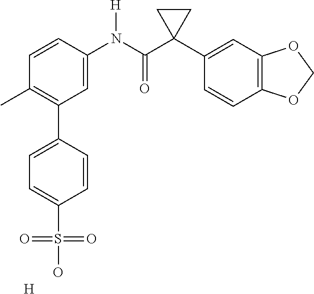 | 1122 |
| 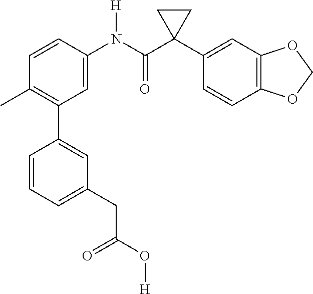 | 1123 |
| 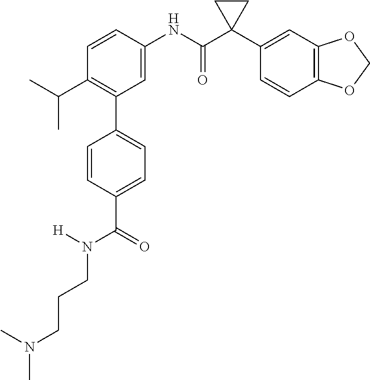 | 1124 |
| 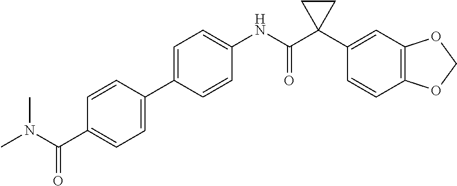 [SMILES: CN(C)C(=O)c1ccc(-c2ccc(NC(=O)C3(c4ccc5c(c4)OCO5)CC3)cc2)cc1] | 1125 |
| 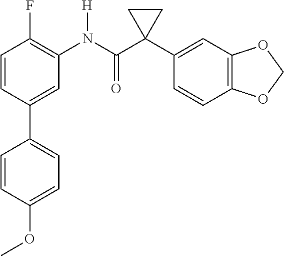 | 1126 |
| 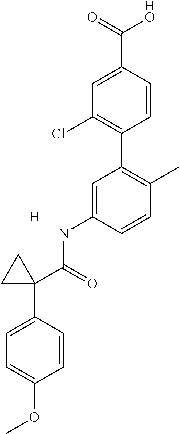 | 1127 |
| 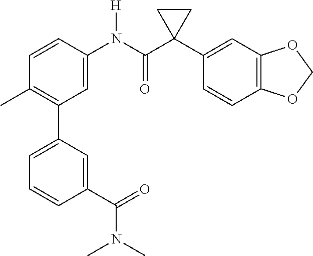 | 1128 |
| 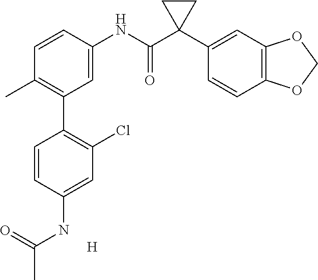 | 1129 |
| 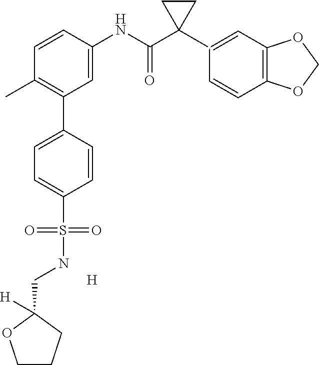 | 1130 |
| 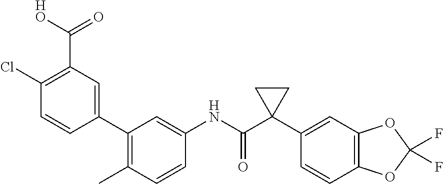 [SMILES: Cc1ccc(NC(=O)C2(c3ccc4c(c3)OC(F)(F)O4)CC2)cc1-c1ccc(Cl)c(C(=O)O)c1] | 1131 |
| 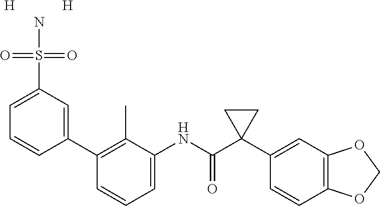 | 1132 |
| 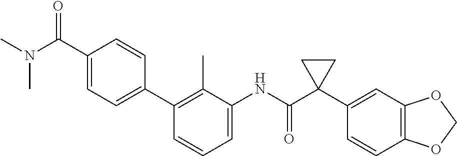 [SMILES: Cc1c(NC(=O)C2(c3ccc4c(c3)OCO4)CC2)cccc1-c1ccc(C(=O)N(C)C)cc1] | 1133 |
| 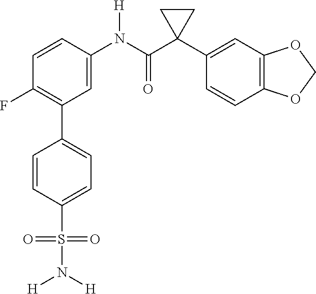 [SMILES: NS(=O)(=O)c1ccc(-c2cc(NC(=O)C3(c4ccc5c(c4)OCO5)CC3)ccc2F)cc1] | 1134 |
| 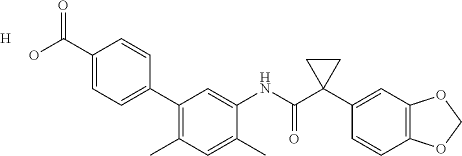 | 1135 |
| 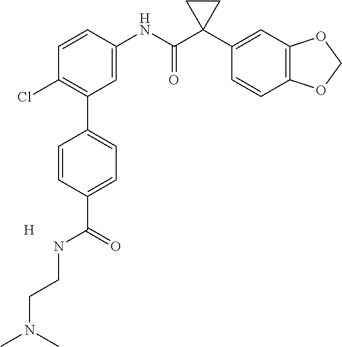 | 1136 |
| 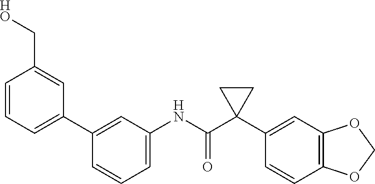 [SMILES: O=C(Nc1cccc(-c2cccc(CO)c2)c1)C1(c2ccc3c(c2)OCO3)CC1] | 1137 |
| 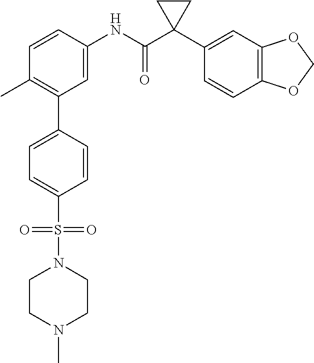 [SMILES: Cc1ccc(NC(=O)C2(c3ccc4c(c3)OCO4)CC2)cc1-c1ccc(S(=O)(=O)N2CCN(C)CC2)cc1] | 1138 |
| 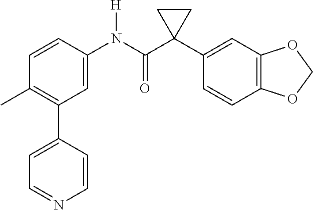 | 1139 |
| 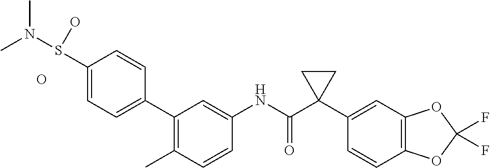 | 1140 |
| 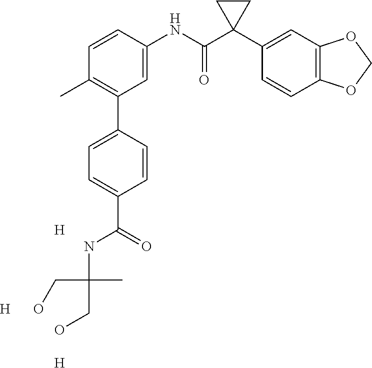 | 1141 |
| 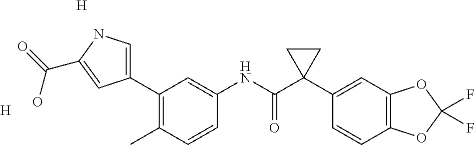 | 1142 |
| 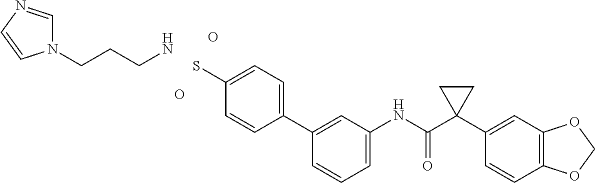 | 1143 |
| 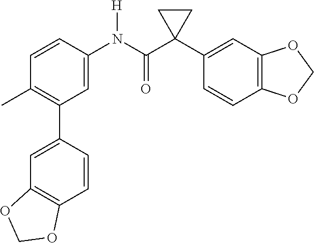 [SMILES: Cc1ccc(NC(=O)C2(c3ccc4c(c3)OCO4)CC2)cc1-c1ccc2c(c1)OCO2] | 1144 |
| 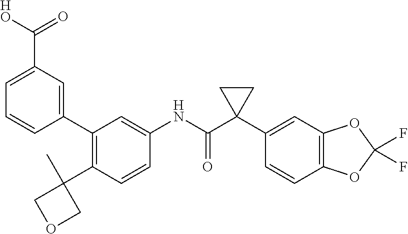 [SMILES: CC1(c2ccc(NC(=O)C3(c4ccc5c(c4)OC(F)(F)O5)CC3)cc2-c2cccc(C(=O)O)c2)COC1] | 1145 |
| 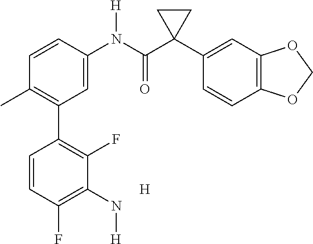 | 1146 |
| 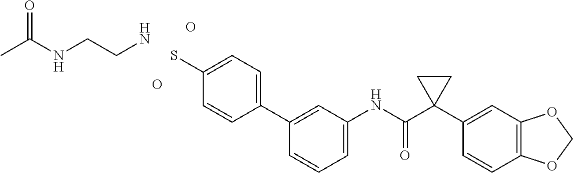 | 1147 |
| 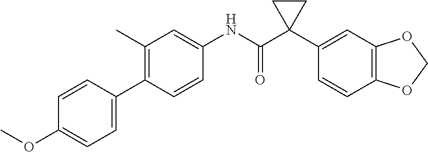 [SMILES: COc1ccc(-c2ccc(NC(=O)C3(c4ccc5c(c4)OCO5)CC3)cc2C)cc1] | 1148 |
| 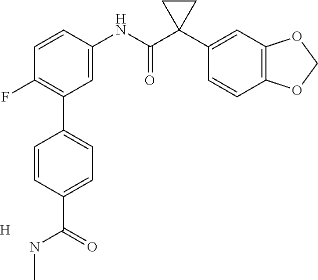 | 1149 |
| 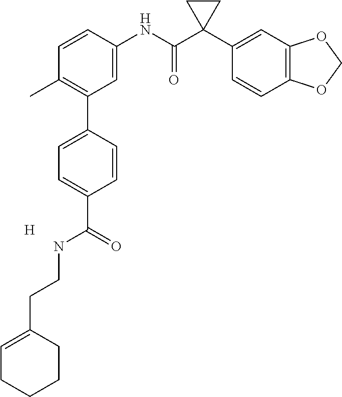 | 1150 |
| 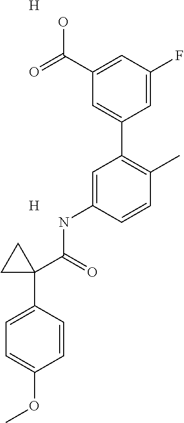 | 1151 |
| 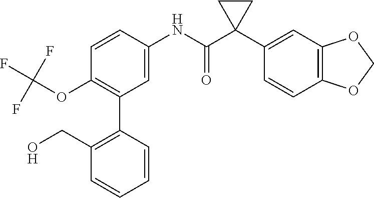 | 1152 |
| 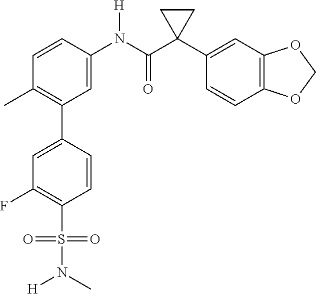 [SMILES: CNS(=O)(=O)c1ccc(-c2cc(NC(=O)C3(c4ccc5c(c4)OCO5)CC3)ccc2C)cc1F] | 1153 |
| 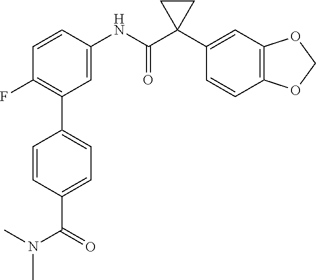 [SMILES: CN(C)C(=O)c1ccc(-c2cc(NC(=O)C3(c4ccc5c(c4)OCO5)CC3)ccc2F)cc1] | 1154 |
| 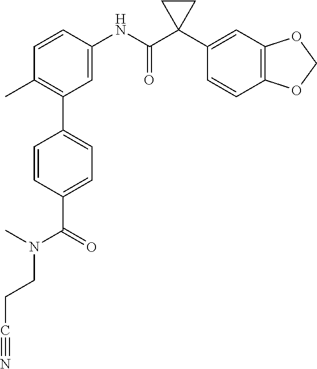 [SMILES: Cc1ccc(NC(=O)C2(c3ccc4c(c3)OCO4)CC2)cc1-c1ccc(C(=O)N(C)CCC#N)cc1] | 1155 |
| 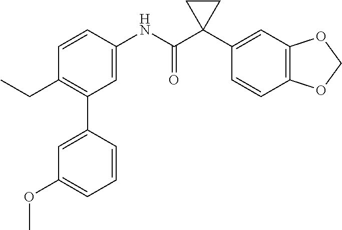 [SMILES: CCc1ccc(NC(=O)C2(c3ccc4c(c3)OCO4)CC2)cc1-c1cccc(OC)c1] | 1156 |
| 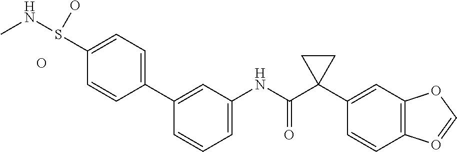 | 1157 |
| 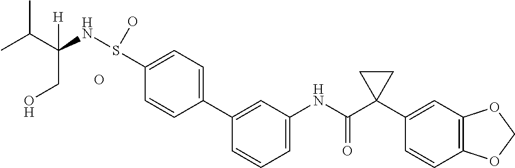 | 1158 |
| 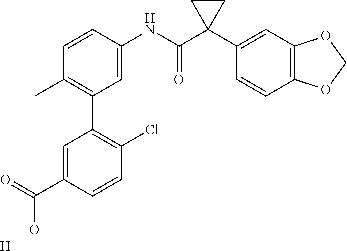 | 1159 |
| 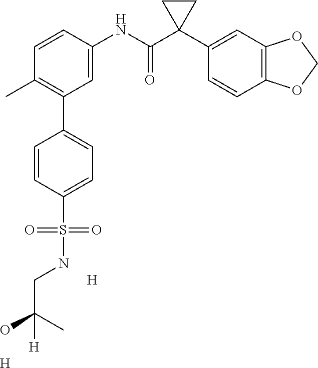 | 1160 |
| 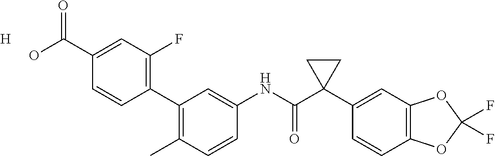 | 1161 |
| 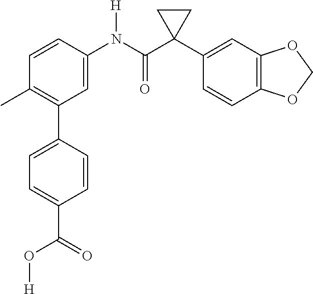 [SMILES: Cc1ccc(NC(=O)C2(c3ccc4c(c3)OCO4)CC2)cc1-c1ccc(C(=O)O)cc1] | 1162 |
| 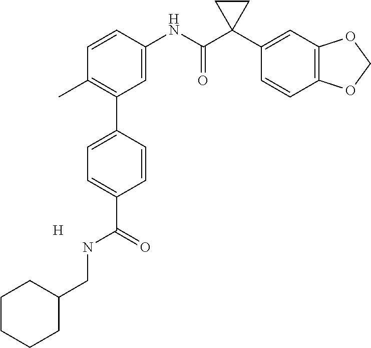 | 1163 |
| 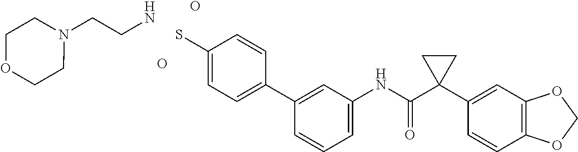 | 1164 |
| 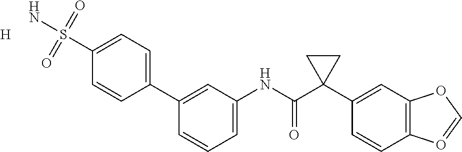 | 1165 |
| 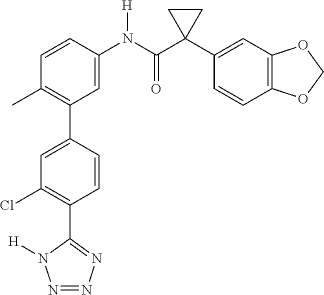 [SMILES: Cc1ccc(NC(=O)C2(c3ccc4c(c3)OCO4)CC2)cc1-c1ccc(-c2nnn[nH]2)c(Cl)c1] | 1166 |
| 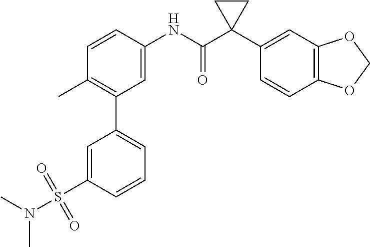 [SMILES: Cc1ccc(NC(=O)C2(c3ccc4c(c3)OCO4)CC2)cc1-c1cccc(S(=O)(=O)N(C)C)c1] | 1167 |
| 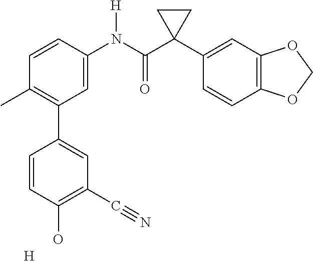 | 1168 |
| 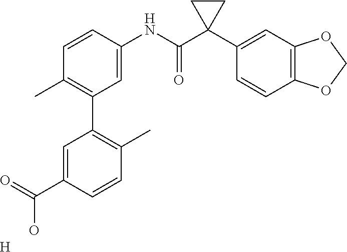 | 1169 |
| 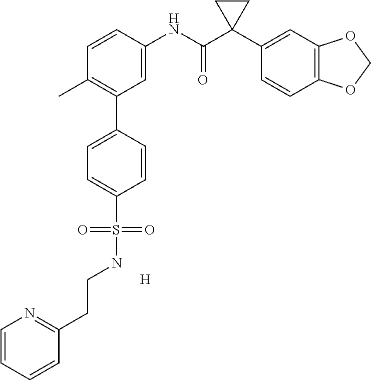 | 1170 |
| 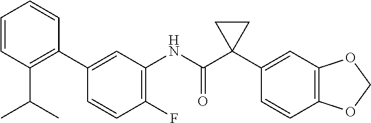 [SMILES: CC(C)c1ccccc1-c1ccc(F)c(NC(=O)C2(c3ccc4c(c3)OCO4)CC2)c1] | 1171 |
| 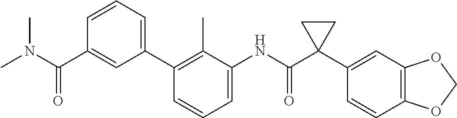 [SMILES: Cc1c(NC(=O)C2(c3ccc4c(c3)OCO4)CC2)cccc1-c1cccc(C(=O)N(C)C)c1] | 1172 |
| 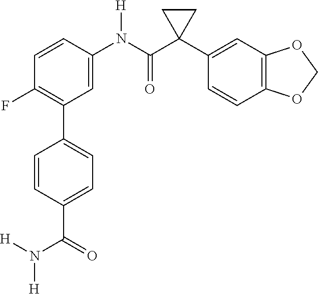 [SMILES: NC(=O)c1ccc(-c2cc(NC(=O)C3(c4ccc5c(c4)OCO5)CC3)ccc2F)cc1] | 1173 |
| 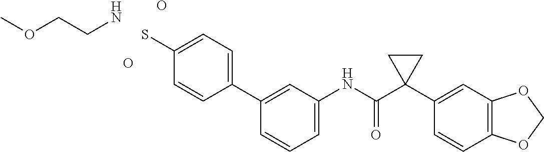 | 1174 |
| 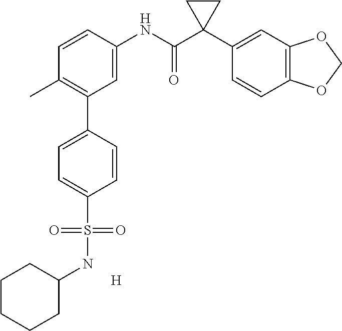 | 1175 |
| 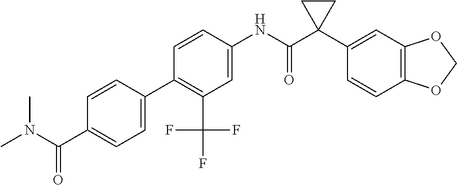 [SMILES: CN(C)C(=O)c1ccc(-c2ccc(NC(=O)C3(c4ccc5c(c4)OCO5)CC3)cc2C(F)(F)F)cc1] | 1176 |
| 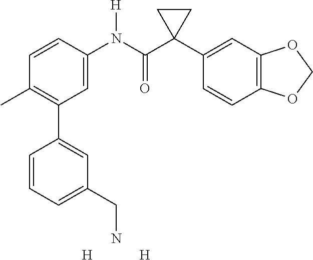 | 1177 |
| 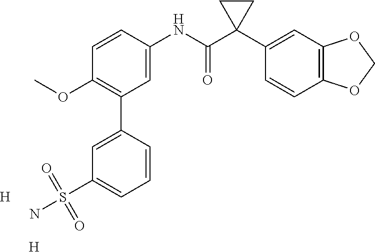 | 1178 |
| 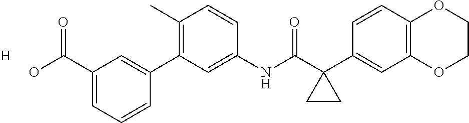 | 1179 |
| 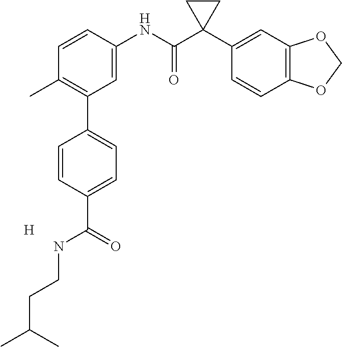 | 1180 |
| 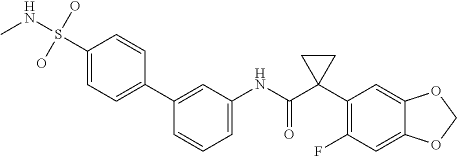 [SMILES: CNS(O)(O)c1ccc(-c2cccc(NC(=O)C3(c4cc5c(cc4F)OCO5)CC3)c2)cc1] | 1181 |
| 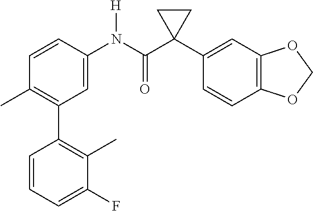 [SMILES: Cc1ccc(NC(=O)C2(c3ccc4c(c3)OCO4)CC2)cc1-c1cccc(F)c1C] | 1182 |
| 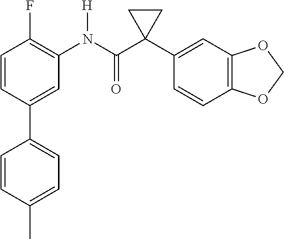 [SMILES: Cc1ccc(-c2ccc(F)c(NC(=O)C3(c4ccc5c(c4)OCO5)CC3)c2)cc1] | 1183 |
| 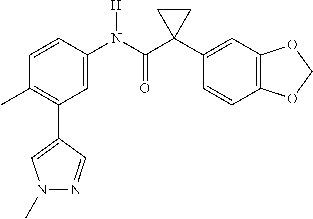 | 1184 |
| 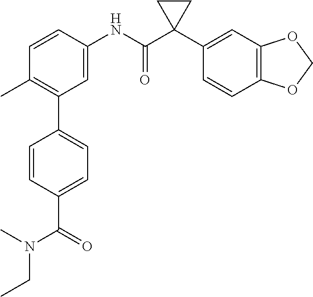 | 1185 |
| 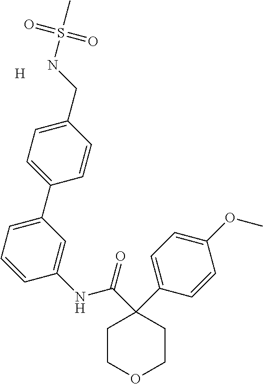 | 1186 |
| 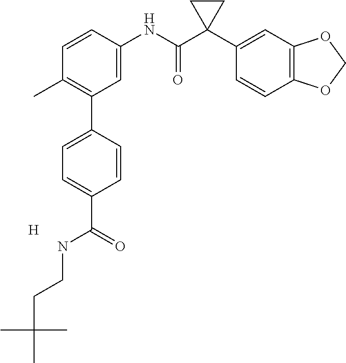 | 1187 |
| 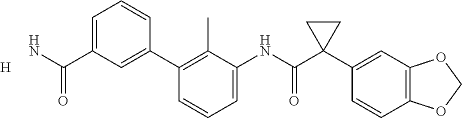 | 1188 |
| 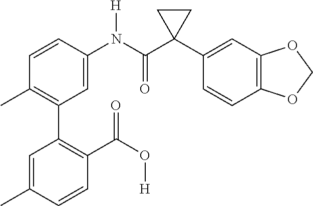 [SMILES: Cc1ccc(C(=O)O)c(-c2cc(NC(=O)C3(c4ccc5c(c4)OCO5)CC3)ccc2C)c1] | 1189 |
| 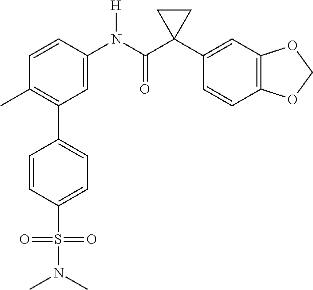 [SMILES: Cc1ccc(NC(=O)C2(c3ccc4c(c3)OCO4)CC2)cc1-c1ccc(S(=O)(=O)N(C)C)cc1] | 1190 |
| 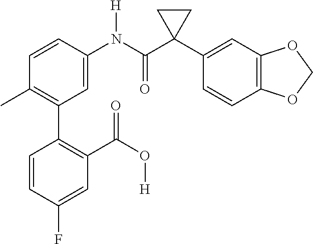 [SMILES: Cc1ccc(NC(=O)C2(c3ccc4c(c3)OCO4)CC2)cc1-c1ccc(F)cc1C(=O)O] | 1192 |
| 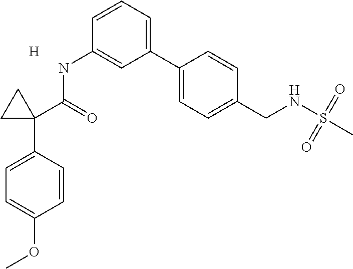 | 1193 |
| 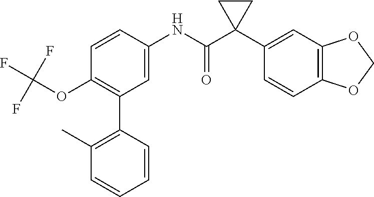 [SMILES: Cc1ccccc1-c1cc(NC(=O)C2(c3ccc4c(c3)OCO4)CC2)ccc1OC(F)(F)F] | 1194 |
| 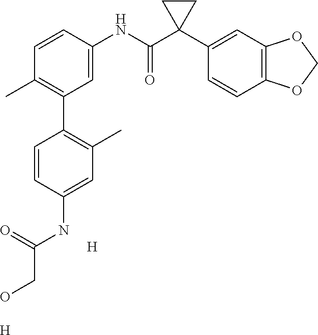 | 1195 |
| 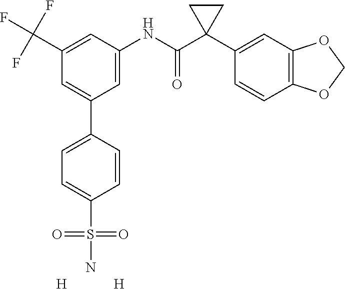 | 1196 |
| 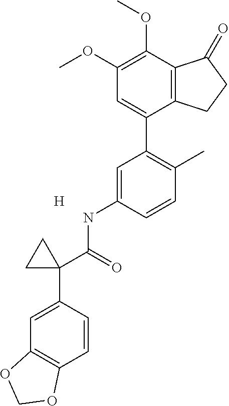 | 1197 |
| 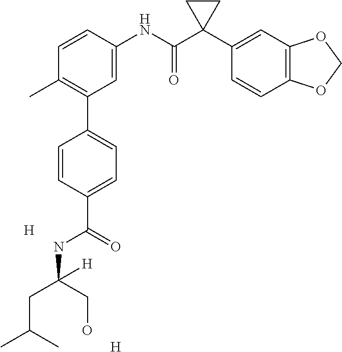 | 1198 |
| 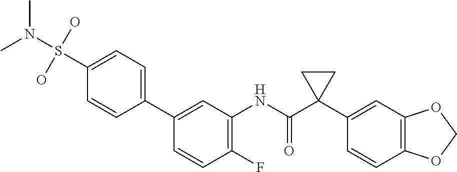 [SMILES: CN(C)S(O)(O)c1ccc(-c2ccc(F)c(NC(=O)C3(c4ccc5c(c4)OCO5)CC3)c2)cc1] | 1199 |
| 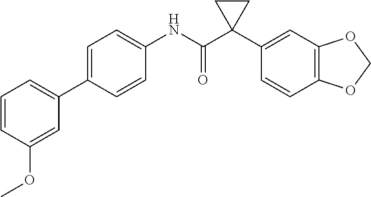 [SMILES: COc1cccc(-c2ccc(NC(=O)C3(c4ccc5c(c4)OCO5)CC3)cc2)c1] | 1200 |
| 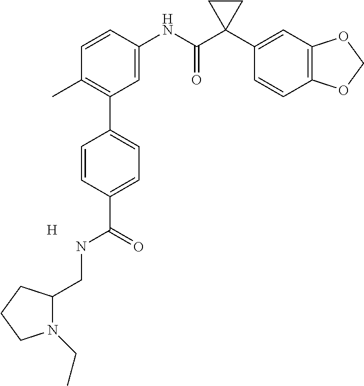 | 1201 |
| 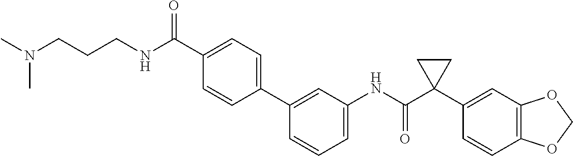 [SMILES: CN(C)CCCNC(=O)c1ccc(-c2cccc(NC(=O)C3(c4ccc5c(c4)OCO5)CC3)c2)cc1] | 1202 |
| 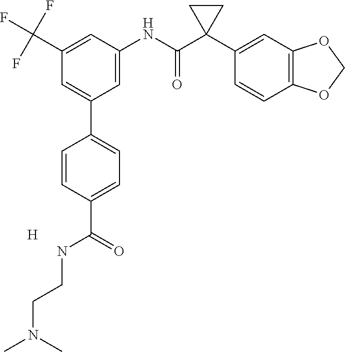 | 1203 |
| 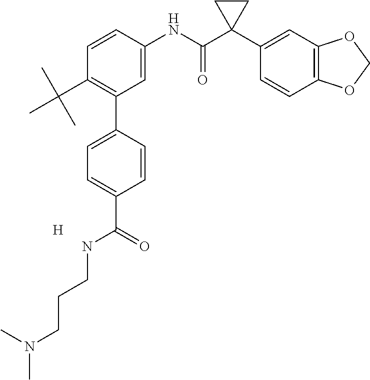 | 1204 |
| 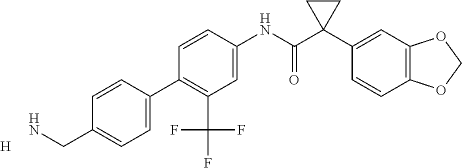 | 1205 |
| 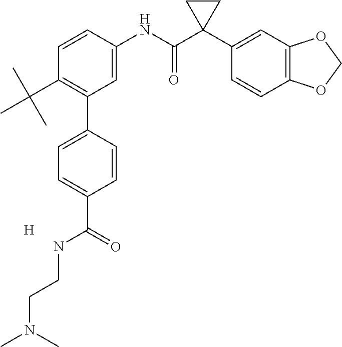 | 1191 |
| 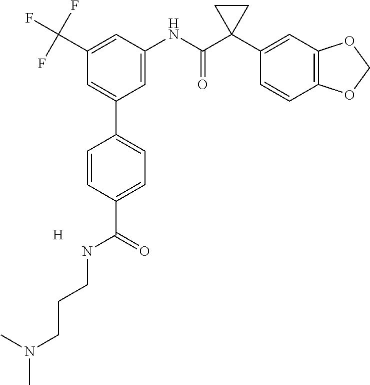 | 959 |
| 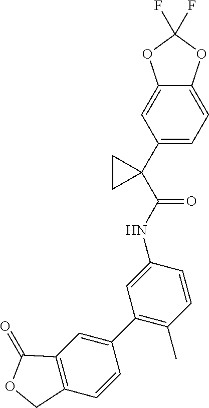 [SMILES: Cc1ccc(NC(=O)C2(c3ccc4c(c3)OC(F)(F)O4)CC2)cc1-c1ccc2c(c1)C(=O)OC2] | 1206 |
| 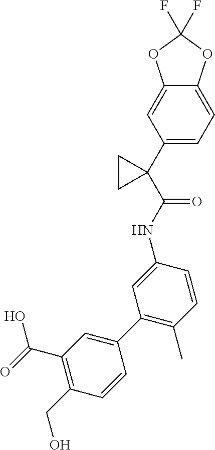 [SMILES: Cc1ccc(NC(=O)C2(c3ccc4c(c3)OC(F)(F)O4)CC2)cc1-c1ccc(CO)c(C(=O)O)c1] | 1207 |
| 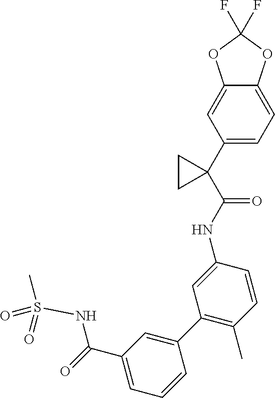 [SMILES: Cc1ccc(NC(=O)C2(c3ccc4c(c3)OC(F)(F)O4)CC2)cc1-c1cccc(C(=O)NS(C)(=O)=O)c1] | 1208 |
| 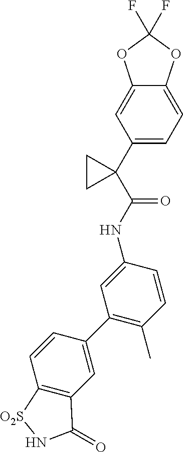 [SMILES: Cc1ccc(NC(=O)C2(c3ccc4c(c3)OC(F)(F)O4)CC2)cc1-c1ccc2c(c1)C(=O)NS2(=O)=O] | 1209 |
| 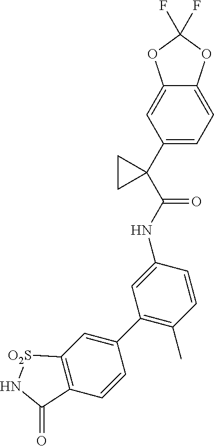 [SMILES: Cc1ccc(NC(=O)C2(c3ccc4c(c3)OC(F)(F)O4)CC2)cc1-c1ccc2c(c1)S(=O)(=O)NC2=O] | 1210 |
| 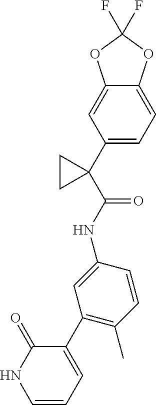 [SMILES: Cc1ccc(NC(=O)C2(c3ccc4c(c3)OC(F)(F)O4)CC2)cc1-c1ccc[nH]c1=O] | 1211 |
| 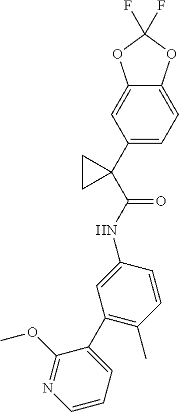 [SMILES: COc1ncccc1-c1cc(NC(=O)C2(c3ccc4c(c3)OC(F)(F)O4)CC2)ccc1C] | 1212 |
| 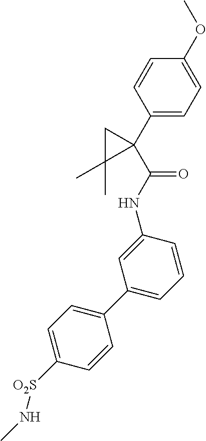 [SMILES: CNS(=O)(=O)c1ccc(-c2cccc(NC(=O)C3(c4ccc(OC)cc4)CC3(C)C)c2)cc1] | 1213 |
| 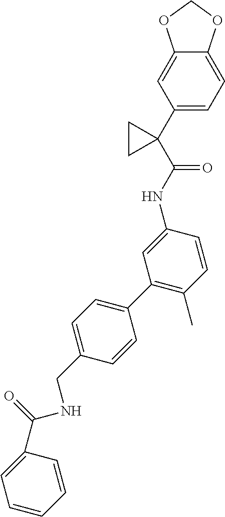 [SMILES: Cc1ccc(NC(=O)C2(c3ccc4c(c3)OCO4)CC2)cc1-c1ccc(CNC(=O)c2ccccc2)cc1] | 1214 |
| 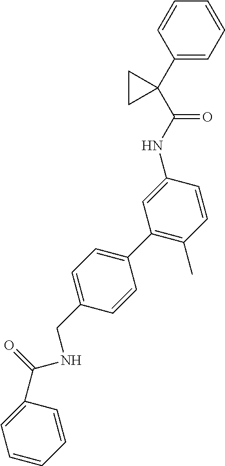 [SMILES: Cc1ccc(NC(=O)C2(c3ccccc3)CC2)cc1-c1ccc(CNC(=O)c2ccccc2)cc1] | 1215 |
| 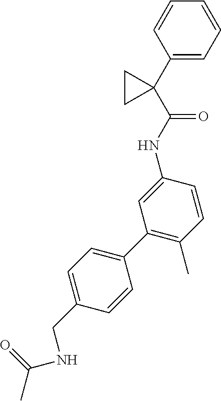 [SMILES: CC(=O)NCc1ccc(-c2cc(NC(=O)C3(c4ccccc4)CC3)ccc2C)cc1] | 1216 |

### Synthetic Schemes

Compounds of the invention may be prepared by well-known methods in the art. Exemplary methods are illustrated below in Scheme I-Scheme IV.

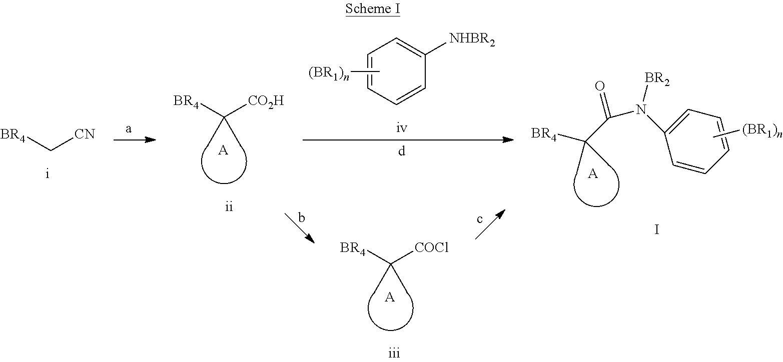

Referring to Scheme I, a nitrile of formula i is alkylated (step a) with a dihalo-aliphatic in the presence of a base such as, for example, 50% sodium hydroxide and, optionally, a phase transfer reagent such as, for example, benzyltriethylammonium chloride (BTEAC), to produce the corresponding alkylated nitrile (not shown) which on hydrolysis in situ produces the acid ii. Compounds of formula ii may be converted to the acid chloride iii (step b) with a suitable reagent such as, for example, thionyl chloride/DMF. Reaction of the acid chloride iii with an aniline of formula iv under known conditions, (step c) produces the amide compounds of the invention formula I. Alternatively, the acid ii may be reacted directly with the aniline iv (step d) in the presence of a coupling reagent such as, for example, HATU, under known conditions to give the amides I.

In some instances, when one of BR1 is a halogen (X in formula v), compounds of Formula B may be further modified as shown below in Scheme II.

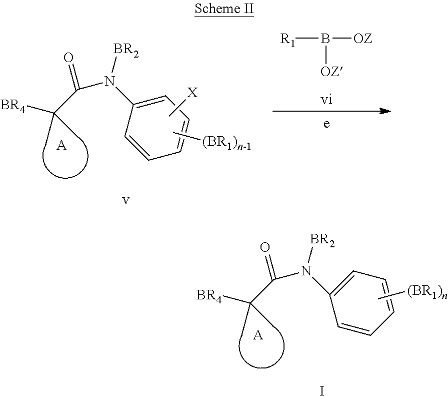

Referring to Scheme II, reaction of the amide v, wherein X is halogen, with a boronic acid derivative vi (step e) wherein Z and Z′ are independently H, alkyl or Z and Z′ together with the atoms to which they are bound form a five or six membered optionally substituted cycloaliphatic ring, in the presence of a catalyst such as, for example, palladium acetate or dichloro-[1,1-bis(diphenylphosphino) ferrocene]palladium(II) (Pd(dppf)Cl2), provides compounds of the invention wherein one of BR1 is aryl or heteroaryl.

The phenylacetonitriles of formula i are commercially available or may be prepared as shown in Scheme III.

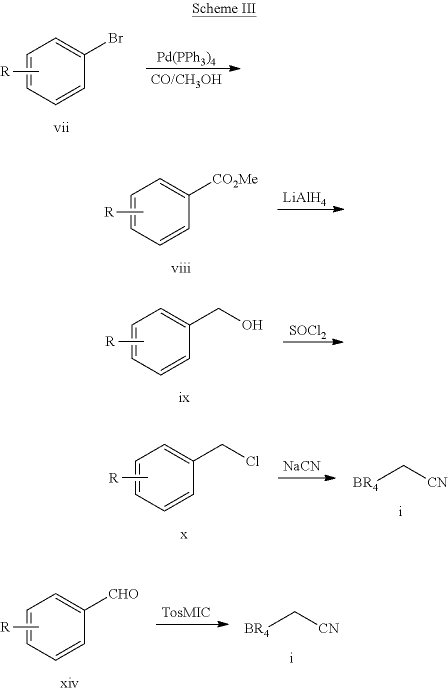

Referring to Scheme III, wherein R represents substituents as described for BR4, the aryl bromide vii is converted to the ester viii with carbon monoxide and methanol in the presence of tetrakis(triphenylphosphine)palladium (0). The ester viii is reduced to the alcohol ix with a reducing reagent such as lithium aluminum hydride. The benzyl alcohol ix is converted to the corresponding benzylchloride with, for example, thionyl chloride. Reaction of the benzylchloride x with a cyanide, for example sodium cyanide, provides the starting nitriles i. Or the aldehyde xiv can also be converted into the corresponding nitrile i by reaction with TosMIC reagent.

The aryl bromides vii are commercially available or may be prepared by known methods.

In some instances, the anilines iv (Scheme I) wherein one of BR1 is aryl or heteroaryl may be prepared as shown in Scheme IV.

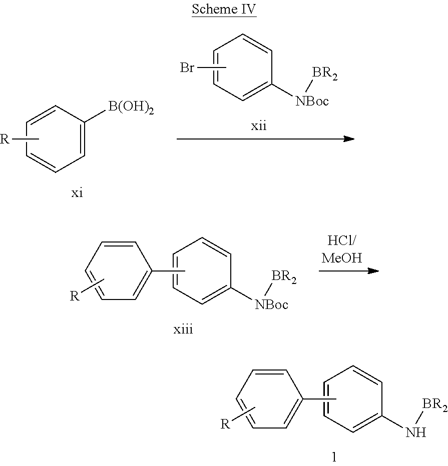

Referring to Scheme IV, an aryl boronic acid xi is coupled with an aniline xii protected as, for example, a tert-butoxycarbonyl derivative (BOC), in the presence of a palladium reagent as previously described for Scheme II to give xiii. Removal of the protecting group under known conditions such as aqueous HCl provides the desired substituted aniline.

Boronic acids are commercially available or may be prepared by known methods.

In some instances, BR1 and BR4 may contain functionality such as, for example, a carboxylate, a nitrile or an amine, which may be further modified using known methods. For example, carboxylates may be converted to amides or carbamates; amines may be converted to amides, sulfonamides or carbamates; nitriles may be reduced to amino methyl compounds which in turn may be further converted to amine derivatives.

### Preparations and Examples

### General Procedure 1

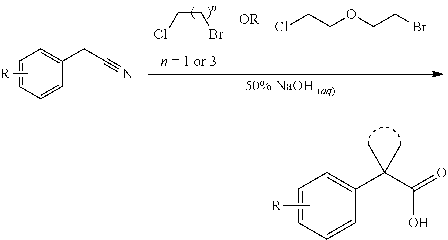

### Preparation 1

### 1-Benzo[1,3]dioxol-5-yl-cyclopropanecarboxylic acid (A-8)

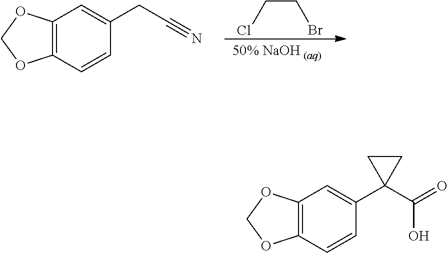

A mixture of benzo[1,3]dioxole-5-acetonitrile (5.10 g 31.7 mmol), 1-bromo-2-chloro-ethane (9.00 mL 109 mmol), and benzyltriethylammonium chloride (0.181 g, 0.795 mmol) was heated at 70° C. and then 50% (wt./wt.) aqueous sodium hydroxide (26 mL) was slowly added to the mixture. The reaction was stirred at 70° C. for 24 hours and was then heated at 130° C. for 48 hours. The dark brown reaction mixture was diluted with water (400 mL) and extracted once with an equal volume of ethyl acetate and once with an equal volume of dichloromethane. The basic aqueous solution was acidified with concentrated hydrochloric acid to pH less than one and the precipitate was filtered and washed with 1 M hydrochloric acid. The solid material was dissolved in dichloromethane (400 mL) and extracted twice with equal volumes of 1 M hydrochloric acid and once with a saturated aqueous solution of sodium chloride. The organic solution was dried over sodium sulfate and evaporated to dryness to give a white to slightly off-white solid (5.23 g, 80%) ESI-MS m/z calc. 206.1. found 207.1 (M+1)+. Retention time 2.37 minutes. 1H NMR (400 MHz, DMSO-d6) δ 1.07-1.11 (m, 2H), 1.38-1.42 (m, 2H), 5.98 (s, 2H), 6.79 (m, 2H), 6.88 (m, 1H), 12.26 (s, 1H).

### Preparation 2

### 1-(2,2-Difluoro-benzo[1,3]dioxol-5-yl)-cyclopropanecarboxylic acid (A-9)

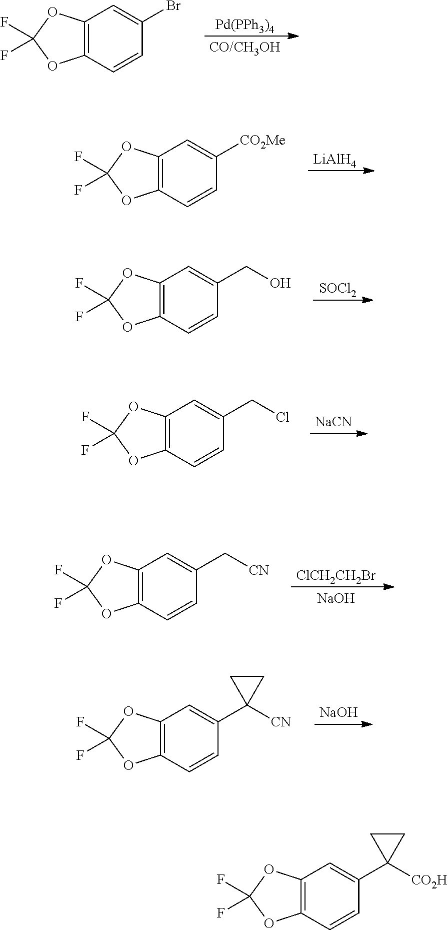

### Step a: 2,2-Difluoro-benzo[1,3]dioxole-5-carboxylic acid methyl ester

A solution of 5-bromo-2,2-difluoro-benzo[1,3]dioxole (11.8 g, 50.0 mmol) and tetrakis(triphenylphosphine)palladium (0) [Pd(PPh3)4, 5.78 g, 5.00 mmol] in methanol (20 mL) containing acetonitrile (30 mL) and triethylamine (10 mL) was stirred under a carbon monoxide atmosphere (55 PSI) at 75° C. (oil bath temperature) for 15 hours. The cooled reaction mixture was filtered and the filtrate was evaporated to dryness. The residue was purified by silica gel column chromatography to give crude 2,2-difluoro-benzo[1,3]dioxole-5-carboxylic acid methyl ester (11.5 g), which was used directly in the next step.

### Step b: (2,2-Difluoro-benzo[1,3]dioxol-5-yl)-methanol

Crude 2,2-difluoro-benzo[1,3]dioxole-5-carboxylic acid methyl ester (11.5 g) dissolved in 20 mL of anhydrous tetrahydrofuran (THF) was slowly added to a suspension of lithium aluminum hydride (4.10 g, 106 mmol) in anhydrous THF (100 mL) at 0° C. The mixture was then warmed to room temperature. After being stirred at room temperature for 1 hour, the reaction mixture was cooled to 0° C. and treated with water (4.1 g), followed by sodium hydroxide (10% aqueous solution, 4.1 mL). The resulting slurry was filtered and washed with THF. The combined filtrate was evaporated to dryness and the residue was purified by silica gel column chromatography to give (2,2-difluoro-benzo[1,3]dioxol-5-yl)-methanol (7.2 g, 76% over two steps) as a colorless oil.

### Step c: 5-Chloromethyl-2,2-difluoro-benzo[1,3]dioxole

Thionyl chloride (45 g, 38 mmol) was slowly added to a solution of (2,2-difluoro-benzo[1,3]dioxol-5-yl)-methanol (7.2 g, 38 mmol) in dichloromethane (200 mL) at 0° C. The resulting mixture was stirred overnight at room temperature and then evaporated to dryness. The residue was partitioned between an aqueous solution of saturated sodium bicarbonate (100 mL) and dichloromethane (100 mL). The separated aqueous layer was extracted with dichloromethane (150 mL) and the organic layer was dried over sodium sulfate, filtrated, and evaporated to dryness to give crude 5-chloromethyl-2,2-difluoro-benzo[1,3]dioxole (4.4 g) which was used directly in the next step.

### Step d: (2,2-Difluoro-benzo[1,3]dioxol-5-yl)-acetonitrile

A mixture of crude 5-chloromethyl-2,2-difluoro-benzo[1,3]dioxole (4.4 g) and sodium cyanide (1.36 g, 27.8 mmol) in dimethylsulfoxide (50 mL) was stirred at room temperature overnight. The reaction mixture was poured into ice and extracted with ethyl acetate (300 mL). The organic layer was dried over sodium sulfate and evaporated to dryness to give crude (2,2-difluoro-benzo[1,3]dioxol-5-yl)-acetonitrile (3.3 g) which was used directly in the next step.

### Step e: 1-(2,2-Difluoro-benzo[1,3]dioxol-5-yl)-cyclopropanecarbonitrile

Sodium hydroxide (50% aqueous solution, 10 mL) was slowly added to a mixture of crude (2,2-difluoro-benzo[1,3]dioxol-5-yl)-acetonitrile, benzyltriethylammonium chloride (3.00 g, 15.3 mmol), and 1-bromo-2-chloroethane (4.9 g, 38 mmol) at 70° C. The mixture was stirred overnight at 70° C. before the reaction mixture was diluted with water (30 mL) and extracted with ethyl acetate. The combined organic layers were dried over sodium sulfate and evaporated to dryness to give crude 1-(2,2-difluoro-benzo[1,3]dioxol-5-yl)-cyclopropanecarbonitrile, which was used directly in the next step.

### Step f: 1-(2,2-Difluoro-benzo[1,3]dioxol-5-yl)-cyclopropanecarboxylic acid (A-9)

To 1-(2,2-difluoro-benzo[1,3]dioxol-5-yl)-cyclopropanecarbonitrile (crude from the last step) was added 10% aqueous sodium hydroxide (50 mL) and the mixture was heated at reflux for 2.5 hours. The cooled reaction mixture was washed with ether (100 mL) and the aqueous phase was acidified to pH 2 with 2M hydrochloric acid. The precipitated solid was filtered to give 1-(2,2-difluoro-benzo[1,3]dioxol-5-yl)-cyclopropanecarboxylic acid as a white solid (0.15 g, 2% over four steps). ESI-MS m/z calc. 242.2. found 243.3; ¹H NMR (CDCl₃) δ 7.14-7.04 (m, 2H), 6.98-6.96 (m, 1H), 1.74-1.64 (m, 2H), 1.26-1.08 (m, 2H).

### Preparation 3

### 2-(4-(Benzyloxy)-3-chlorophenyl)acetonitrile

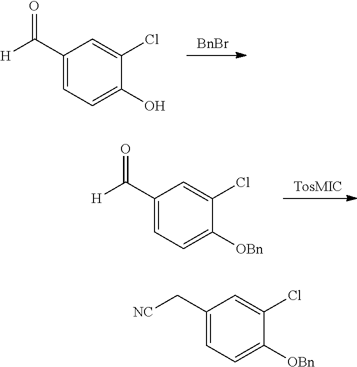

### Step a: 4-Benzyloxy-3-chloro-benzaldehyde

To a solution of 3-chloro-4-hydroxy-benzaldehyde (5.0 g, 32 mmol) and BnBr (6.6 g, 38 mmol) in CH3CN (100 mL) was added K2CO3 (8.8 g, 64 mmol). The mixture was heated at reflux for 2 hours. The resulting mixture was poured into water (100 mL), and extracted with EtOAc (100 mL×3). The combined organic layers were washed with brine, dried over anhydrous Na2SO4 and evaporated under vacuum to give crude product, which was purified by column (petroleum ether/EtOAc 15:1) to give 4-benzyloxy-3-chloro-benzaldehyde (7.5 g, 95%). 1H NMR (CDCl3, 400 MHz) δ 9.85 (s, 1H), 7.93 (d, J=2.0 Hz, 1H), 7.73 (dd, J=2.0, 8.4 Hz, 1H), 7.47-7.34 (m, 5H), 7.08 (d, J=8.8 Hz, 1H), 4.26 (s, 2H).

### Step b: 2-(4-(Benzyloxy)-3-chlorophenyl)acetonitrile

To a suspension of t-BuOK (11.7 g, 96 mmol) in THF (200 mL) was added a solution of TosMIC (9.4 g, 48 mmol) in THF (100 mL) at −78° C. The mixture was stirred for 15 minutes, treated with a solution of 4-benzyloxy-3-chloro-benzaldehyde (7.5 g, 30 mmol) in THF (50 mL) dropwise, and continued to stir for 1.5 hours at −78° C. To the cooled reaction mixture was added methanol (30 mL). The mixture was heated at reflux for 30 minutes. Solvent of the reaction mixture was removed to give a crude product, which was dissolved in water (300 mL). The aqueous phase was extracted with EtOAc (3×100 mL). The combined organic layers were dried and evaporated under reduced pressure to give crude product, which was purified by column chromatography (petroleum ether/EtOAc 10:1) to afford 2-(4-(benzyloxy)-3-chlorophenyl)acetonitrile (2.7 g, 34%). 1H NMR (400 MHz, CDCl3) δ 7.52-7.32 (m, 6H), 7.15 (dd, J=2.4, 8.4 Hz, 1H), 6.95 (d, J=8.4 Hz, 1H), 5.26 (s, 2H), 3.73 (s, 2H). ¹³C NMR (100 MHz, CDCl3) δ 154.0, 136.1, 129.9, 128.7, 128.7, 128.1, 127.2, 127.1, 127.1, 124.0, 123.0, 117.5, 114.4, 70.9, 22.5.

### Preparation 4

### 1-(2-Oxo-2,3-dihydrobenzo[d]oxazol-5-yl)cyclopropane-carboxylic acid (A-19)

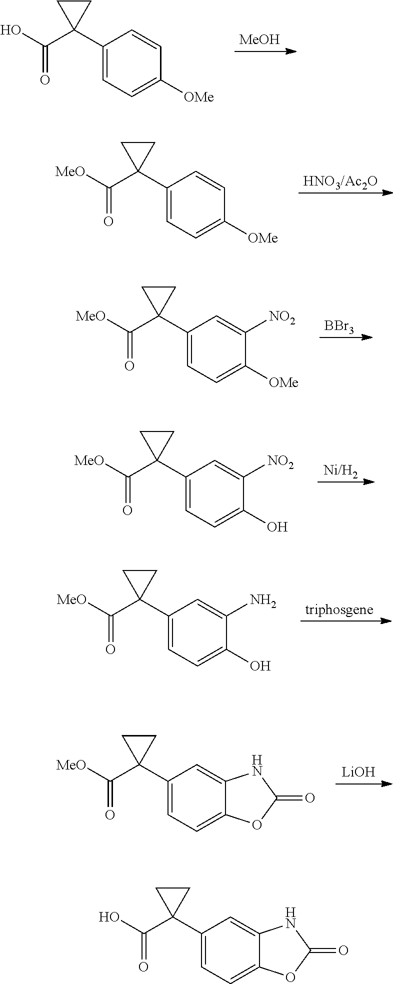

### Step a: 1-(4-Methoxy-phenyl)-cyclopropanecarboxylic acid methyl ester

To a solution of 1-(4-methoxy-phenyl)-cyclopropanecarboxylic acid (50.0 g, 0.26 mol) in MeOH (500 mL) was added toluene-4-sulfonic acid monohydrate (2.5 g, 13.1 mmol) at room temperature. The reaction mixture was heated at reflux for 20 hours. MeOH was removed by evaporation under vacuum and EtOAc (200 mL) was added. The organic layer was washed with sat. aq. NaHCO3 (100 mL) and brine, dried over anhydrous Na2SO4 and evaporated under vacuum to give 1-(4-methoxy-phenyl)-cyclopropanecarboxylic acid methyl ester (53.5 g, 99%). 1H NMR (CDCl3, 400 MHz) δ 7.25-7.27 (m, 2H), 6.85 (d, J=8.8 Hz, 2H), 3.80 (s, 3H), 3.62 (s, 3H), 1.58 (q, J=3.6 Hz, 2H), 1.15 (q, J=3.6 Hz, 2H).

### Step b: 1-(4-Methoxy-3-nitro-phenyl)-cyclopropanecarboxylic acid methyl ester

To a solution of 1-(4-methoxy-phenyl)-cyclopropanecarboxylic acid methyl ester (30.0 g, 146 mmol) in Ac2O (300 mL) was added a solution of HNO3 (14.1 g, 146 mmol, 65%) in AcOH (75 mL) at 0° C. The reaction mixture was stirred at 0˜5° C. for 3 h before aq. HCl (20%) was added dropwise at 0° C. The resulting mixture was extracted with EtOAc (200 mL×3). The organic layer was washed with sat. aq. NaHCO3 then brine, dried over anhydrous Na2SO4 and evaporated under vacuum to give 1-(4-methoxy-3-nitro-phenyl)-cyclopropanecarboxylic acid methyl ester (36.0 g, 98%), which was directly used in the next step. ¹H NMR (CDCl3, 300 MHz) δ 7.84 (d, J=2.1 Hz, 1H), 7.54 (dd, J=2.1, 8.7 Hz, 1H), 7.05 (d, J=8.7 Hz, 1H), 3.97 (s, 3H), 3.65 (s, 3H), 1.68-1.64 (m, 2H), 1.22-1.18 (m, 2H).

### Step c: 1-(4-Hydroxy-3-nitro-phenyl)-cyclopropanecarboxylic acid methyl ester

To a solution of 1-(4-methoxy-3-nitro-phenyl)-cyclopropane-carboxylic acid methyl ester (10.0 g, 39.8 mmol) in CH2Cl2 (100 mL) was added BBr3 (12.0 g, 47.8 mmol) at −70° C. The mixture was stirred at −70° C. for 1 hour, then allowed to warm to −30° C. and stirred at this temperature for 3 hours. Water (50 mL) was added dropwise at −20° C., and the resulting mixture was allowed to warm room temperature before it was extracted with EtOAc (200 mL×3). The combined organic layers were dried over anhydrous Na2SO4 and evaporated under vacuum to give the crude product, which was purified by column chromatography on silica gel (petroleum ether/EtOAc 15:1) to afford 1-(4-hydroxy-3-nitro-phenyl)-cyclopropanecarboxylic acid methyl ester (8.3 g, 78%). 1H NMR (CDCl3, 400 MHz) δ 10.5 (s, 1H), 8.05 (d, J=2.4 Hz, 1H), 7.59 (dd, J=2.0, 8.8 Hz, 1H), 7.11 (d, J=8.4 Hz, 1H), 3.64 (s, 3H), 1.68-1.64 (m, 2H), 1.20-1.15 (m, 2H).

### Step d: 1-(3-Amino-4-hydroxy-phenyl)-cyclopropanecarboxylic acid methyl ester

To a solution of 1-(4-hydroxy-3-nitro-phenyl)-cyclopropanecarboxylic acid methyl ester (8.3 g, 35.0 mmol) in MeOH (100 mL) was added Raney Ni (0.8 g) under nitrogen atmosphere. The mixture was stirred under hydrogen atmosphere (1 atm) at 35° C. for 8 hours. The catalyst was filtered off through a Celite pad and the filtrate was evaporated under vacuum to give crude product, which was purified by column chromatography on silica gel (P.E./EtOAc 1:1) to give 1-(3-amino-4-hydroxy-phenyl)-cyclopropanecarboxylic acid methyl ester (5.3 g, 74%). 1H NMR (CDCl3, 400 MHz) δ 6.77 (s, 1H), 6.64 (d, J=2.0 Hz, 2H), 3.64 (s, 3H), 1.55-1.52 (m, 2H), 1.15-1.12 (m, 2H).

### Step e: 1-(2-Oxo-2,3-dihydro-benzooxazol-5-yl)-cyclopropanecarboxylic acid methyl ester

To a solution of 1-(3-amino-4-hydroxy-phenyl)-cyclopropanecarboxylic acid methyl ester (2.0 g, 9.6 mmol) in THF (40 mL) was added triphosgene (4.2 g, 14 mmol) at room temperature. The mixture was stirred for 20 minutes at this temperature before water (20 mL) was added dropwise at 0° C. The resulting mixture was extracted with EtOAc (100 mL×3). The combined organic layers were dried over anhydrous Na2SO4 and evaporated under vacuum to give 1-(2-oxo-2,3-dihydro-benzooxazol-5-yl)-cyclopropanecarboxylic acid methyl ester (2.0 g, 91%), which was directly used in the next step. 1H NMR (CDCl3, 300 MHz) δ 8.66 (s, 1H), 7.13-7.12 (m, 2H), 7.07 (s, 1H), 3.66 (s, 3H), 1.68-1.65 (m, 2H), 1.24-1.20 (m, 2H).

### Step f: 1-(2-Oxo-2,3-dihydrobenzo[d]oxazol-5-yl)cyclopropanecarboxylic acid

To a solution of 1-(2-oxo-2,3-dihydro-benzooxazol-5-yl)-cyclopropanecarboxylic acid methyl ester (1.9 g, 8.1 mmol) in MeOH (20 mL) and water (2 mL) was added LiOH.H2O (1.7 g, 41 mmol) in portions at room temperature. The reaction mixture was stirred for 20 hours at 50° C. MeOH was removed by evaporation under vacuum before water (100 mL) and EtOAc (50 mL) were added. The aqueous layer was separated, acidified with HCl (3 mol/L) and extracted with EtOAc (100 mL×3). The combined organic layers were dried over anhydrous Na2SO4 and evaporated under vacuum to give 1-(2-oxo-2,3-dihydrobenzo[d]oxazol-5-yl)cyclopropanecarboxylic acid (1.5 g, 84%). 1H NMR (DMSO, 400 MHz) δ 12.32 (brs, 1H), 11.59 (brs, 1H), 7.16 (d, J=8.4 Hz, 1H), 7.00 (d, J=8.0 Hz, 1H), 1.44-1.41 (m, 2H), 1.13-1.10 (m, 2H). MS (ESI) m/e (M+H+) 218.1.

### Preparation 5

### 1-(Benzo[d]oxazol-5-yl)cyclopropanecarboxylic acid (A-20)

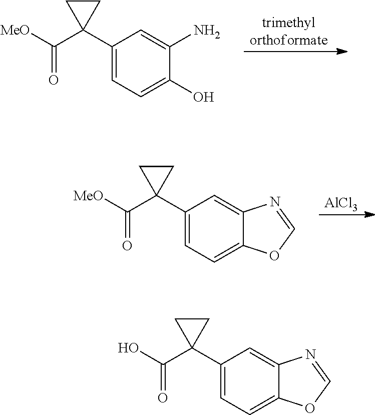

### Step a: 1-Benzooxazol-5-yl-cyclopropanecarboxylic acid methyl ester

To a solution of 1-(3-amino-4-hydroxy-phenyl)-cyclopropanecarboxylic acid methyl ester (3.00 g, 14.5 mmol) in DMF were added trimethyl orthoformate (5.30 g, 14.5 mmol) and a catalytic amount of p-tolueneslufonic acid monohydrate (0.3 g) at room temperature. The mixture was stirred for 3 hours at room temperature. The mixture was diluted with water and extracted with EtOAc (100 mL×3). The combined organic layers were dried over anhydrous Na2SO4 and evaporated under vacuum to give crude 1-benzooxazol-5-yl-cyclopropanecarboxylic acid methyl ester (3.1 g), which was directly used in the next step. 1H NMR (CDCl3, 400 MHz) δ 8.09 (s, 1), 7.75 (d, J=1.2 Hz, 1H), 7.53-7.51 (m, 1H), 7.42-7.40 (m, 1H), 3.66 (s, 3H), 1.69-1.67 (m, 2H), 1.27-1.24 (m, 2H).

### Step b: 1-(Benzo[d]oxazol-5-yl)cyclopropanecarboxylic acid

To a solution of crude 1-benzooxazol-5-yl-cyclopropanecarboxylic acid methyl ester (2.9 g) in EtSH (30 mL) was added AlCl3 (5.3 g, 40.1 mmol) in portions at 0° C. The reaction mixture was stirred for 18 hours at room temperature. Water (20 mL) was added dropwise at 0° C. The resulting mixture was extracted with EtOAc (100 mL×3). The combined organic layers were dried over anhydrous Na2SO4 and evaporated under vacuum to give the crude product, which was purified by column chromatography on silica gel (petroleum ether/EtOAc 1:2) to give 1-(benzo[d]oxazol-5-yl)cyclopropanecarboxylic acid (280 mg, two steps: 11%). 1H NMR (DMSO, 400 MHz) δ 12.25 (brs, 1H), 8.71 (s, 1H), 7.70-7.64 (m, 2H), 7.40 (dd, J=1.6, 8.4 Hz, 1H), 1.49-1.46 (m, 2H), 1.21-1.18 (m, 2H). MS (ESI) m/e (M+H+) 204.4.

### Preparation 6

### 2-(7-Chlorobenzo[d][1,3]dioxol-5-yl)acetonitrile

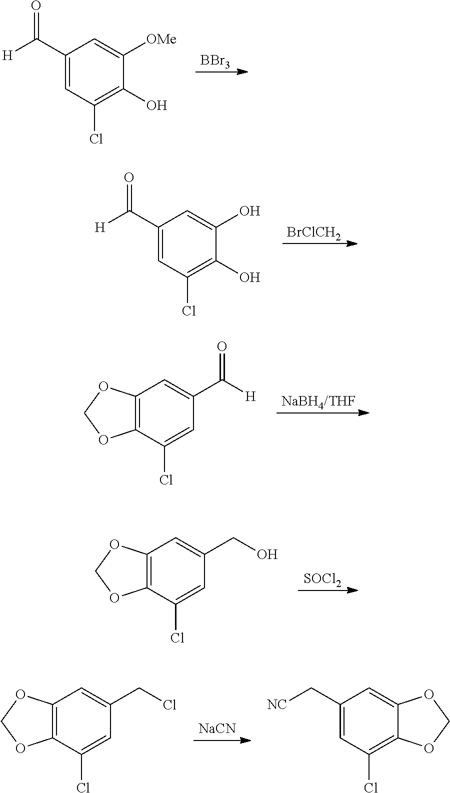

### Step a: 3-Chloro-4,5-dihydroxybenzaldehyde

To a suspension of 3-chloro-4-hydroxy-5-methoxy-benzaldehyde (10 g, 54 mmol) in dichloromethane (300 mL) was added BBr3 (26.7 g, 107 mmol) dropwise at −40° C. under N2. After addition, the mixture was stirred at this temperature for 5 h and then was poured into ice water. The precipitated solid was filtered and washed with petroleum ether. The filtrate was evaporated under reduced pressure to afford 3-chloro-4,5-dihydroxybenzaldehyde (9.8 g, 89%), which was directly used in the next step.

### Step b: 7-Chlorobenzo[d][1,3]dioxole-5-carbaldehyde

To a solution of 3-chloro-4,5-dihydroxybenzaldehyde (8.0 g, 46 mmol) and BrClCH2 (23.9 g, 185 mmol) in dry DMF (100 mL) was added Cs2CO3 (25 g, 190 mmol). The mixture was stirred at 60° C. overnight and was then poured into water. The resulting mixture was extracted with EtOAc (50 mL×3). The combined extracts were washed with brine (100 mL), dried over Na2SO4 and concentrated under reduced pressure to afford 7-chlorobenzo[d][1,3]dioxole-5-carbaldehyde (6.0 g, 70%). 1H NMR (400 MHz, CDCl3) δ 9.74 (s, 1H), 7.42 (d, J=0.4 Hz, 1H), 7.26 (d, J=3.6 Hz, 1H), 6.15 (s, 2H)

### Step c: (7-Chlorobenzo[d][1,3]dioxol-5-yl)methanol

To a solution of 7-chlorobenzo[d][1,3]dioxole-5-carbaldehyde (6.0 g, 33 mmol) in THF (50 mL) was added NaBH4 (2.5 g, 64 mmol)) in portion at 0° C. The mixture was stirred at this temperature for 30 min and then poured into aqueous NH4Cl solution. The organic layer was separated, and the aqueous phase was extracted with EtOAc (50 mL×3). The combined extracts were dried over Na2SO4 and evaporated under reduced pressure to afford (7-chlorobenzo[d][1,3]dioxol-5-yl)methanol, which was directly used in the next step.

### Step d: 4-Chloro-6-(chloromethyl)benzo[d][1,3]dioxole

A mixture of (7-chlorobenzo[d][1,3]dioxol-5-yl)methanol (5.5 g, 30 mmol) and SOCl2 (5.0 mL, 67 mmol) in dichloromethane (20 mL) was stirred at room temperature for 1 h and was then poured into ice water. The organic layer was separated and the aqueous phase was extracted with dichloromethane (50 mL×3). The combined extracts were washed with water and aqueous NaHCO3 solution, dried over Na2SO4 and evaporated under reduced pressure to afford 4-chloro-6-(chloromethyl)benzo[d][1,3]dioxole, which was directly used in the next step.

### Step e: 2-(7-Chlorobenzo[d][1,3]dioxol-5-yl)acetonitrile

A mixture of 4-chloro-6-(chloromethyl)benzo[d][1,3]dioxole (6.0 g, 29 mmol) and NaCN (1.6 g, 32 mmol) in DMSO (20 mL) was stirred at 40° C. for 1 h and was then poured into water. The mixture was extracted with EtOAc (30 mL×3). The combined organic layers were washed with water and brine, dried over Na2SO4 and evaporated under reduced pressure to afford 2-(7-chlorobenzo[d][1,3]dioxol-5-yl)acetonitrile (3.4 g, 58%). 1H NMR δ 6.81 (s, 1H), 6.71 (s, 1H), 6.07 (s, 2H), 3.64 (s, 2H). 13C-NMR 8149.2, 144.3, 124.4, 122.0, 117.4, 114.3, 107.0, 102.3, 23.1.

### Preparation 7

### 2-(7-Fluorobenzo[d][1,3]dioxol-5-yl)acetonitrile

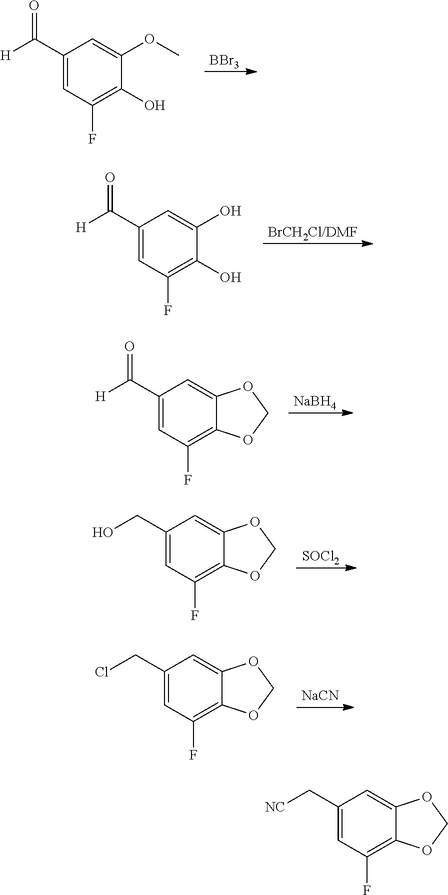

### Step a: 3-Fluoro-4,5-dihydroxy-benzaldehyde

To a suspension of 3-fluoro-4-hydroxy-5-methoxy-benzaldehyde (1.35 g, 7.94 mmol) in dichloromethane (100 mL) was added BBr3 (1.5 mL, 16 mmol) dropwise at −78° C. under N2. After addition, the mixture was warmed to −30° C. and it was stirred at this temperature for 5 h. The reaction mixture was poured into ice water. The precipitated solid was collected by filtration and washed with dichloromethane to afford 3-fluoro-4,5-dihydroxy-benzaldehyde (1.1 g, 89%), which was directly used in the next step.

### Step b: 7-Fluoro-benzo[1,3]dioxole-5-carbaldehyde

To a solution of 3-fluoro-4,5-dihydroxy-benzaldehyde (1.5 g, 9.6 mmol) and BrClCH2 (4.9 g, 38.5 mmol) in dry DMF (50 mL) was added Cs2CO3 (12.6 g, 39 mmol). The mixture was stirred at 60° C. overnight and was then poured into water. The resulting mixture was extracted with EtOAc (50 mL×3). The combined organic layers were washed with brine (100 mL), dried over Na2SO4 and evaporated under reduced pressure to give the crude product, which was purified by column chromatography on silica gel (petroleum ether/E.A.=10/1) to afford 7-fluoro-benzo[1,3]dioxole-5-carbaldehyde (0.80 g, 49%). 1H NMR (300 MHz, CDCl3) δ 9.78 (d, J=0.9 Hz, 1H), 7.26 (dd, J=1.5, 9.3 Hz, 1H), 7.19 (d, J=1.2 Hz, 1H), 6.16 (s, 2H).

### Step c: (7-Fluoro-benzo[1,3]dioxol-5-yl)-methanol

To a solution of 7-fluoro-benzo[1,3]dioxole-5-carbaldehyde (0.80 g, 4.7 mmol) in MeOH (50 mL) was added NaBH4 (0.36 g, 9.4 mmol) in portions at 0° C. The mixture was stirred at this temperature for 30 min and was then concentrated to dryness. The residue was dissolved in EtOAc. The EtOAc layer was washed with water, dried over Na2SO4 and concentrated to dryness to afford (7-fluoro-benzo[1,3]dioxol-5-yl)-methanol (0.80 g, 98%), which was directly used in the next step.

### Step d: 6-Chloromethyl-4-fluoro-benzo[1,3]dioxole

To SOCl2 (20 mL) was added (7-fluoro-benzo[1,3]dioxol-5-yl)-methanol (0.80 g, 4.7 mmol) in portions at 0° C. The mixture was warmed to room temperature over 1 h and then was heated at reflux for 1 h. The excess SOCl2 was evaporated under reduced pressure to give the crude product, which was basified with saturated aqueous NaHCO3 to pH ˜7. The aqueous phase was extracted with EtOAc (50 mL×3). The combined organic layers were dried over Na2SO4 and evaporated under reduced pressure to give 6-chloromethyl-4-fluoro-benzo[1,3]dioxole (0.80 g, 92%), which was directly used in the next step.

### Step e: 2-(7-Fluorobenzo[d][1,3]dioxol-5-yl)acetonitrile

A mixture of 6-chloromethyl-4-fluoro-benzo[1,3]dioxole (0.80 g, 4.3 mmol) and NaCN (417 mg, 8.51 mmol) in DMSO (20 mL) was stirred at 30° C. for 1 h and was then poured into water. The mixture was extracted with EtOAc (50 mL×3). The combined organic layers were washed with water (50 mL) and brine (50 mL), dried over Na2SO4 and evaporated under reduced pressure to give the crude product, which was purified by column chromatography on silica gel (petroleum ether/E.A.=10/1) to afford 2-(7-fluorobenzo[d][1,3]dioxol-5-yl)acetonitrile (530 mg, 70%). 1H NMR (300 MHz, CDCl3) δ 6.68-6.64 (m, 2H), 6.05 (s, 2H), 3.65 (s, 2H). 13C-NMR 5151.1, 146.2, 134.1, 124.2, 117.5, 110.4, 104.8, 102.8, 23.3.

Additional acids given in Table II.B-2 were either commercially available or synthesized using appropriate starting materials and the procedures of preparations 1-7.

TABLE II
B-2: Carboxylic Acids.

| Acids | Name |
|---|---|
| A-1 | 1-Phenylcyclopropanecarboxylic acid |
| A-2 | 1-(2-Methoxyphenyl)cyclopropanecarboxylic acid |
| A-3 | 1-(3-Methoxyphenyl)cyclopropanecarboxylic acid |
| A-4 | 1-(4-Methoxyphenyl)cyclopropanecarboxylic acid |
| A-5 | 1-(4-(Trifluoromethoxy)phenyl)cyclopropanecarboxylic acid |
| A-6 | 1-(4-Chlorophenyl)cyclopropanecarboxylic acid |
| A-7 | 1-(3,4-Dimethoxyphenyl)cyclopropanecarboxylic acid |
| A-8 | 1-Benzo[1,3]dioxol-5-yl-cyclopropanecarboxylic acid |
| A-9 | 1-(2,2-Difluoro-benzo[1,3]dioxol-5-yl)-cyclopropanecarboxylic |
| | acid |
| A-10 | 1-Phenylcyclopentanecarboxylic acid |
| A-11 | 1-(4-Chlorophenyl)cyclopentanecarboxylic acid |
| A-12 | 1-(4-Methoxyphenyl)cyclopentanecarboxylic acid |
| A-13 | 1-(Benzo[d][1,3]dioxol-5-yl)cyclopentanecarboxylic acid |
| A-14 | 1-Phenylcyclohexanecarboxylic acid |
| A-15 | 1-(4-Chlorophenyl)cyclohexanecarboxylic acid |
| A-16 | 1-(4-Methoxyphenyl)cyclohexanecarboxylic acid |
| A-17 | 4-(4-Methoxyphenyl)tetrahydro-2H-pyran-4-carboxylic acid |
| A-18 | 1-(3-Chloro-4-hydroxyphenyl)cyclopropanecarboxylic acid |
| A-19 | 1-(2-Oxo-2,3-dihydrobenzo[d]oxazol-5-yl) |
| | cyclopropanecarboxylic acid |
| A-20 | 1-(Benzo[d]oxazol-5-yl)cyclopropanecarboxylic acid |
| A-21 | 1-(7-Chlorobenzo[d][1,3]dioxol-5-yl)cyclopentanecarboxylic acid |
| A-22 | 1-(7-Fluorobenzo[d][1,3]dioxol-5-yl)cyclopentanecarboxylic acid |
| A-23 | 1-(3,4-Difluorophenyl)cyclopropanecarboxylic acid |
| A-24 | 1-(1H-Indol-5-yl)cyclopropanecarboxylic acid |
| A-25 | 1-(2,3-Dihydrobenzo[b][1,4]dioxin-6-yl)cyclopropanecarboxylic |
| | acid |
| A-26 | 1-(2,3-Dihydrobenzofuran-5-yl)cyclopropanecarboxylic acid |
| A-27 | 1-(3,4-Dichlorophenyl)cyclopropanecarboxylic acid |
| A-28 | 1-(2-Methyl-1H-benzo[d]imidazol-5-yl)cyclopropanecarboxylic |
| | acid |
| A-29 | 1(4-Hydroxy-4-methoxychroman-6-yl)cyclopropanecarboxylic |
| | acid |
| A-30 | 1-(Benzofuran-6-yl)cyclopropanecarboxylic acid |
| A-31 | 1-(1-Methyl-1H-benzo[d][1,2,3]triazol-5- |
| | yl)cyclopropanecarboxylic acid |
| A-32 | 1-(2,3-Dihydrobenzofuran-6-yl)cyclopropanecarboxylic acid |
| A-33 | 1(3-Methylbenzo[d]isoxazol-5-yl)cyclopropanecarboxylic acid |
| A-34 | 1-(4-Oxochroman-6-yl)cyclopropanecarboxylic acid |
| A-35 | 1-(Spiro[benzo[d][1,3]dioxole-2,1′-cyclobutane]-5- |
| | yl)cyclopropanecarboxylic acid |
| A-36 | 1-(1,3-Dihydroisobenzofuran-5-yl)cyclopropanecarboxylic acid |
| A-37 | 1(6-Fluorobenzo[d][1,3]dioxol-5-yl)cyclopropanecarboxylic acid |
| A-38 | 1-(Chroman-6-yl)cyclopropanecarboxylic acid |

### Preparation 8

### 3-Bromo-4-methoxybenzenamine

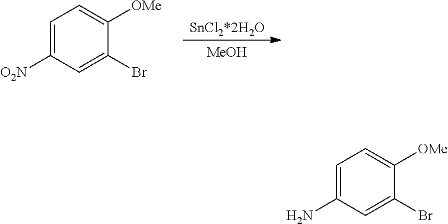 [SMILES: "MeOH" ; COc1ccc([N+](=O)[O-])cc1Br ; COc1ccc(N)cc1Br]

2-Bromo-1-methoxy-4-nitrobenzene (2.50 g, 10.8 mmol), SnCl2.2H2O (12.2 g, 53.9 mmol), and MeOH (30 mL) were combined and allowed to stir for 3 h at ambient temperature. To the mixture was added H2O (100 mL) and EtOAc (100 mL) resulting in the formation of a thick emulsion. To this was added sat. aq. NaHCO3 (30 mL). The layers were separated and the aqueous layer was extracted with EtOAc (3×30 mL). The organics were combined and dried over MgSO4 before being filtered. Concentration of the filtrate in vacuo gave 2.02 g of an off-white solid. This material was used without further purification.

In addition to bromo-anilines prepared according to preparation 8, non-limiting examples of commercially available bromo anilines and bromo nitrobenzenes are given in Table II.B-3.

TABLE II
B-3: Non-limiting examples of commercially available anilines.
Name
4-Bromoaniline
4-Bromo-3-methylaniline
4-Bromo-3-(trifluoromethyl)aniline
3-Bromoaniline
5-Bromo-2-methylaniline
5-Bromo-2-fluoroaniline
5-Bromo-2-(trifluoromethoxy)aniline
3-Bromo-4-methylaniline
3-Bromo-4-fluoroaniline
2-Bromo-1-methoxy-4-nitrobenzene
2-Bromo-1-chloro-4-nitrobenzene
4-Bromo-3-methylaniline
3-Bromo-4-methylaniline
3-Bromo-4-(trifluoromethoxy)aniline
3-Bromo-5-(trifluoromethyl)aniline
3-Bromo-2-methylaniline

### Preparation 9

### 1-(Benzo[d][1,3]dioxol-5-yl)-N-(3-bromo-4-methoxyphenyl)cyclopropane-carboxamide (B-10)

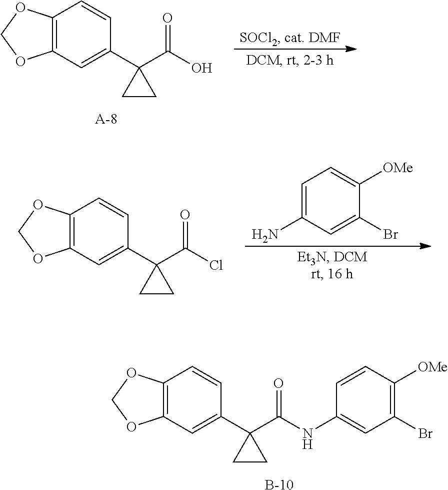

### Step a: 1-Benzo[1,3]dioxol-5-yl-cyclopropanecarbonyl chloride

To an oven-dried round bottom flask containing 1-(benzo[d][1,3]dioxol-5-yl)-cyclopropanecarboxylic acid (A-8) (618 mg, 3.0 mmol) and CH₂Cl₂ (3 mL) was added thionyl chloride (1.07 g, 9.0 mmol) and N,N-dimethylformamide (0.1 mL). The reaction mixture was stirred at ambient temperature under an Ar atmosphere until the gas evolution ceased (2-3 h). The excess thionyl chloride was removed under vacuum and the resulting residue dissolved in CH₂Cl₂ (3 mL). The mixture was used without further manipulation.

### Step b: 1-(Benzo[d][1,3]dioxol-5-yl)-N-(3-bromo-4-methoxyphenyl)-cyclopropane-carboxamide (B-10)

To a solution of the crude 1-benzo[1,3]dioxol-5-yl-cyclopropanecarbonyl chloride (3.0 mmol) in CH2Cl2 (30 mL) at ambient temperature was added a solution of 3-bromo-4-methoxybenzenamine (3.3 mmol), Et3N (15 mmol), and CH2Cl2 (90 mL) dropwise. The mixture was allowed to stir for 16 h before it was diluted with CH2Cl2 (500 mL). The solution was washed with 1N HCl (2×250 mL), sat. aq. NaHCO3 (2×250 mL), then brine (250 mL). The organics were dried over Na2SO4, filtered, and concentrated in vacuo to provide 1-(benzo[d][1,3]dioxol-5-yl)-N-(3-bromo-4-methoxyphenyl)cyclopropanecarboxamide (B-10) with suitable purity to be used without further purification.

TABLE II
B-4 lists additional N-bromophenyl amides prepared according
to preparation 9 and using appropriate starting materials.

| Aryl | | |
|---|---|---|
| bromides | Name | Anilines |
| B-1 | 1-(Benzo[d][1,3]dioxol-5-yl)-N-(4- | 4-Bromoaniline |
| | bromophenyl)cyclopropanecarboxamide | |
| B-2 | 1-(Benzo[d][1,3]dioxol-5-yl)-N-(4-bromo-3- | 4-Bromo-3-methylaniline |
| | methylphenyl)cyclopropanecarboxamide | |
| B-3 | 1-(Benzo[d][1,3]dioxol-5-yl)-N-(4-bromo-3- | 4-Bromo-3- |
| | (trifluoromethyl)phenyl)cyclopropanecarboxamide | (trifluoromethyl)aniline |
| B-4 | 1-(Benzo[d][1,3]dioxol-5-yl)-N-(3- | 3-Bromoaniline |
| | bromophenyl)cyclopropanecarboxamide | |
| B-5 | 1-(Benzo[d][1,3]dioxol-5-yl)-N-(5-bromo-2- | 5-Bromo-2-methylaniline |
| | methylphenyl)cyclopropanecarboxamide | |
| B-6 | 1-(Benzo[d][1,3]dioxol-5-yl)-N-(5-bromo-2- | 5-Bromo-2-fluoroaniline |
| | fluorophenyl)cyclopropanecarboxamide | |
| B-7 | 1-(Benzo[d][1,3]dioxol-5-yl)-N-(5-bromo-2- | 5-Bromo-2- |
| | (trifluoromethoxy)phenyl)cyclopropanecarboxamide | (trifluoromethoxy)aniline |
| B-8 | 1-(Benzo[d][1,3]dioxol-5-yl)-N-(3-bromo-4- | 3-Bromo-4-methylaniline |
| | methylphenyl)cyclopropanecarboxamide | |
| B-9 | 1-(Benzo[d][1,3]dioxol-5-yl)-N-(3-bromo-4- | 3-Bromo-4-fluoroaniline |
| | fluorophenyl)cyclopropanecarboxamide | |
| B-10 | 1-(Benzo[d][1,3]dioxol-5-yl)-N-(3-bromo-4- | 3-Bromo-4- |
| | methoxyphenyl)cyclopropanecarboxamide | methoxybenzenamine |
| B-11 | 1-(Benzo[d][1,3]dioxol-5-yl)-N-(3-bromo-4- | 3-Bromo-4-chloroaniline |
| | chlorophenyl)cyclopropanecarboxamide | |
| B-13 | 1-(Benzo[d][1,3]dioxol-5-yl)-N-(3-bromo-4- | 3-Bromo-4-isopropylaniline |
| | isopropylphenyl)cyclopropanecarboxamide | |
| B-14 | N-(4-Bromo-3-methylphenyl)-1-(2,2- | 4-Bromo-3-methylaniline |
| | difluorobenzo[d][1,3]dioxol-5- | |
| | yl)cyclopropanecarboxamide | |
| B-15 | N-(3-Bromo-4-methylphenyl)-1-(2,2- | 3-Bromo-4-methylaniline |
| | difluorobenzo[d][l,3]dioxol-5- | |
| | yl)cyclopropanecarboxamide | |
| B-16 | 1-(Benzo[d][1,3]dioxol-5-yl)-N-(3-bromo-4-tert- | 3-Bromo-4-tert-butylaniline |
| | butylphenyl)cyclopropanecarboxamide | |
| B-18 | 1-(Benzo[d][1,3]dioxol-5-yl)-N-(3-bromo-4- | 3-Bromo-4-ethylaniline |
| | ethylphenyl)cyclopropanecarboxamide | |
| B-19 | 1-(Benzo[d][1,3]dioxol-5-yl)-N-(3-bromo-4- | 3 -Bromo-4- |
| | (trifluoromethoxy)phenyl)cyclopropanecarboxamide | (trifluoromethoxy)aniline |
| B-20 | 1-(Benzo[d][1,3]dioxol-5-yl)-N-(5-bromo-2-fluoro- | 5-Bromo-2-fluoro-4- |
| | 4-methylphenyl)cyclopropanecarboxamide | methylaniline |
| B-21 | 1-(Benzo[d][1,3]dioxol-5-yl)-N-(3-bromo-5- | 3-Bromo-5- |
| | (trifluoromethyl)phenyl)cyclopropanecarboxamide | (trifluoromethyl)aniline |
| B-22 | 1-(Benzo[d][1,3]dioxol-5-yl)-N-(3-bromo-2- | 3-Bromo-2-methylaniline |
| | methylphenyl)cyclopropanecarboxamide | |
| B-23 | N-(3-Bromo-4-(3-methyloxetan-3-yl)phenyl)-1- | 3-Bromo-4-(3-methyloxetan- |
| | (2,2-difluorobenzo[d][1,3]dioxol-5- | 3-yl)aniline |
| | yl)cyclopropanecarboxamide | |
| B-24 | N-(3-Bromo-4-methylphenyl)-1-(4- | 3-Bromo-4-methylaniline |
| | methoxyphenyl)cyclopropanecarboxamide | |

### Preparation 10

### ((3′-Aminobiphenyl-4-yl)methyl)-methanesulfonamide (C-1)

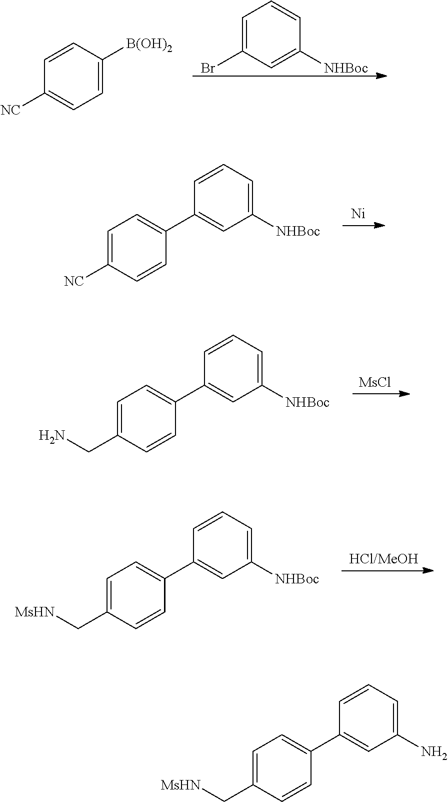

### Step a: (4′-Cyano-biphenyl-3-yl)-carbamic acid tert-butyl ester

A mixture of 4-cyanobenzeneboronic acid (14.7 g, 0.10 mol), 3-bromo-phenyl-carbamic acid tert-butyl ester (27.2 g, 0.10 mol), Pd(Ph3P)4 (11.6 g, 0.01 mol) and K2CO3 (21 g, 0.15 mol) in DMF/H2O (1:1, 350 mL) was stirred under argon at 80° C. overnight. The DMF was evaporated under reduced pressure, and the residue was dissolved in EtOAc (200 mL). The mixture was washed with water and brine, dried over Na2SO4, and concentrated to dryness. The residue was purified by column chromatography (petroleum ether/EtOAc 50:1) on silica gel to give (4′-cyano-biphenyl-3-yl)-carbamic acid tert-butyl ester (17 g, 59%). 1H NMR (300 MHz, DMSO-d6) δ 9.48 (s, 1H), 7.91 (d, J=8.4 Hz, 2H), 7.85 (s, 1H), 7.76 (d, J=8.4 Hz, 2H), 7.32-7.48 (m, 3H), 1.47 (s, 9H).

### Step b: (4′-Aminomethyl-biphenyl-3-yl)-carbamic acid tert-butyl ester

A suspension of (4′-cyano-biphenyl-3-yl)-carbamic acid tert-butyl ester (7.6 g, 26 mmol) and Raney Ni (1 g) in EtOH (500 mL) and NH3.H2O (10 mL) was hydrogenated under 50 psi of H2 at 50° C. for 6 h. The catalyst was filtered off and the filtrate was concentrated to dryness to give (4′-aminomethyl-biphenyl-3-yl)-carbamic acid tert-butyl ester, which was used directly in next step.

### Step c: [4′-(Methanesulfonylamino-methyl)-biphenyl-3-yl]-carbamic acid tert-butyl ester

To a solution of crude (4′-aminomethyl-biphenyl-3-yl)-carbamic acid tert-butyl ester (8.2 g 27 mmol) and Et3N (4.2 g, 40 mmol) in dichloromethane (250 mL) was added dropwise MsCl (3.2 g, 27 mmol) at 0° C. The reaction mixture was stirred at this temperature for 30 min and was then washed with water and saturated aqueous NaCl solution, dried over Na2SO4, and concentrated to dryness. The residue was recrystallized with DCM/pet ether (1:3) to give [4′-(methanesulfonylamino-methyl)-biphenyl-3-yl]-carbamic acid tert-butyl ester (7.5 g, yield 73%). 1H NMR (300 MHz, CDCl3) δ 7.67 (s, 1H), 7.58 (d, J=8.1 Hz, 2H), 7.23-7.41 (m, 5H), 6.57 (s, 1H), 4.65-4.77 (m, 1H), 4.35 (d, J=6 Hz, 2H), 2.90 (s, 3H), 1.53 (s, 9H).

### Step d: N-((3′-Aminobiphenyl-4-yl)methyl)methanesulfonamide

A solution of [4′-(methanesulfonylamino-methyl)-biphenyl-3-yl]-carbamic acid tert-butyl ester (5 g, 13 mmol) in HCl/MeOH (4M, 150 mL) was stirred at room temperature overnight. The mixture was concentrated to dryness and the residue was washed with ether to give the target compound N-((3′-aminobiphenyl-4-yl)methyl)methanesulfonamide as its HCl salt (3.0 g, 71%). 1H NMR (300 MHz, DMSO-d6) δ 7.54-7.71 (m, 6H), 7.46 (d, J=7.8 Hz, 2H), 7.36 (d, J=7.5 Hz, 1H), 4.19 (s, 2H), 2.87 (s, 3H). MS (ESI) m/e (M+H+): 277.0.

### Preparation 11

### (R)-(1-(3′-Aminobiphenyl-4-ylsulfonyl)pyrrolidin-2-yl)methanol (C-2)

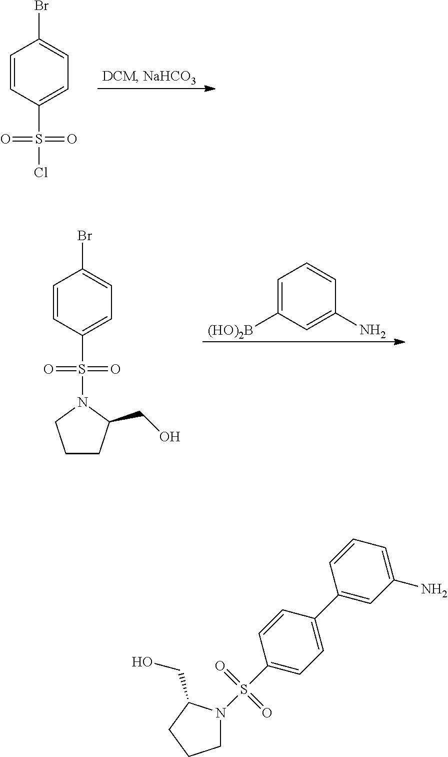 [SMILES: O=S(=O)(Cl)c1ccc(Br)cc1 ; O=S(=O)(c1ccc(Br)cc1)N1CCC[C@@H]1CO ; Nc1cccc(B(O)O)c1 ; Nc1cccc(-c2ccc(S(=O)(=O)N3CCC[C@@H]3CO)cc2)c1]

### Step a: (R)-Bromo-benzenesulfonyl)-pyrrolidin-2-yl]methanol

To a mixture of sat aq. NaHCO3 (44 g, 0.53 mol), CH2Cl2 (400 mL) and (R)-pyrrolidin-2-yl-methanol (53 g, 0.53 mol) was added 4-bromo-benzenesulfonyl chloride (130 g, 0.50 mol) in CH2Cl2 (100 mL). The reaction was stirred at 20° C. overnight. The organic phase was separated and dried over Na2SO4. Evaporation of the solvent under reduced pressure provided (R)-[1-(4-bromo-benzenesulfonyl)-pyrrolidin-2-yl]-methanol (145 g, crude), which was used in the next step without further purification. 1H NMR (CDCl3, 300 MHz) δ 7.66-7.73 (m, 4H), 3.59-3.71 (m, 3H), 3.43-3.51 (m, 1H), 3.18-3.26 (m, 1H), 1.680-1.88 (m, 3H), 1.45-1.53 (m, 1H).

### Step b: (R)-(1-(3′-Aminobiphenyl-4-ylsulfonyl)pyrrolidin-2-yl)methanol (C-2)

To a solution of (R)-[1-(4-bromo-benzenesulfonyl)-pyrrolidin-2-yl]-methanol (1.6 g, 5.0 mmol) in DMF (10 mL) was added 3-amino-phenyl boronic acid (0.75 g, 5.5 mmol), Pd(PPh3)4 (45 mg, 0.15 mmol), potassium carbonate (0.75 g, 5.5 mmol) and water (5 mL). The resulting mixture was degassed by gently bubbling argon through the solution for 5 minutes at 20° C. The reaction mixture was then heated at 80° C. overnight. The reaction was filtered through a pad of silica gel, which was washed with CH2Cl2 (25 mL×3). The combined organics were concentrated under reduced pressure to give the crude product, which was washed with EtOAc to give pure (R)-(1-(3′-aminobiphenyl-4-ylsulfonyl)pyrrolidin-2-yl)methanol (C-2) (810 mg, 49%). 1H NMR (300 MHz, CDCl3) δ 7.88 (d, J=8.7 Hz, 2H), 7.70 (d, J=8.7 Hz, 2H), 7.23-7.28 (m, 1H), 6.98 (d, J=7.8 Hz, 1H), 6.91 (d, J=1.8 Hz, 1H), 6.74 (dd, J=7.8, 1.2 Hz, 1H), 3.66-3.77 (m, 3H), 3.45-3.53 (m, 1H), 3.26-3.34 (m, 1H), 1.68-1.88 (m, 3H), 1.45-1.55 (m, 1H). MS (ESI) m/e (M+H+) 333.0.

### Preparation 12

### 3′-Amino-N-methylbiphenyl-4-sulfonamide (C-3)

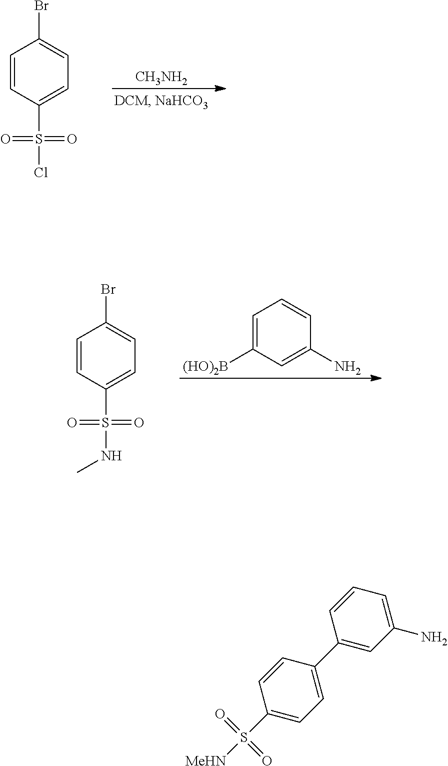 [SMILES: O=S(=O)(Cl)c1ccc(Br)cc1 ; "CH3NH2" ; CNS(=O)(=O)c1ccc(Br)cc1 ; Nc1cccc(B(O)O)c1 ; CNS(=O)(=O)c1ccc(-c2cccc(N)c2)cc1]

### Step a: 4-Bromo-N-methyl-benzenesulfonamide

To a mixture of sat aq. NaHCO3 (42 g, 0.50 mol), CH2Cl2 (400 mL) and methylamine (51.7 g, 0.50 mol, 30% in methanol) was added a solution of 4-bromo-benzenesulfonyl chloride (130 g, 0.50 mol) in CH2Cl2 (100 mL). The reaction was stirred at 20° C. overnight. The organic phase was separated and dried over Na2SO4. Evaporation of the solvent under reduced pressure provided 4-bromo-N-methyl-benzenesulfonamide (121 g, crude), which was used in the next step without further purification. 1H NMR (CDCl3, 300 MHz) δ 7.65-7.74 (m, 4H), 4.40 (br, 1H), 2.67 (d, J=5.4 Hz, 3H).

### Step b: 3′-Amino-N-methylbiphenyl-4-sulfonamide (C-3)

To a solution of 4-bromo-N-methyl-benzene sulfonamide (2.49 g, 10 mmol) in DMF (20 mL) was added 3-amino-phenyl boronic acid (1.51 g, 11 mmol), Pd(PPh3)4 (90 mg, 0.30 mmol), potassium carbonate (1.52 g, 11 mmol) and water (5 mL). The resulting mixture was degassed by gently bubbling argon through the solution for 5 minutes at 20° C. The reaction mixture was then heated at 80° C. overnight. The reaction was filtered through a pad of silica gel, which was washed with CH2Cl2 (50 mL×3). The combined organics were concentrated under reduced pressure to give crude product, which was washed with EtOAc to give pure 3′-amino-N-methylbiphenyl-4-sulfonamide (C-3) (1.3 g, 50%). 1H NMR (300 MHz, CDCl3) δ 7.85 (d, J=8.7 Hz, 2H), 7.75 (d, J=8.7 Hz, 2H), 7.19 (t, J=7.8 Hz, 1H), 6.95-7.01 (m, 2H), 6.73-6.77 (m, 1H), 2.54 (s, 3H). MS (ESI) m/e (M+H+) 263.0.

### Preparation 13

### 5′-(1-(Benzo[d][1,3]dioxol-5-yl)cyclopropanecarboxamido)-2′-(hydroxymethyl)-N,N-dimethylbiphenyl-4-carboxamide

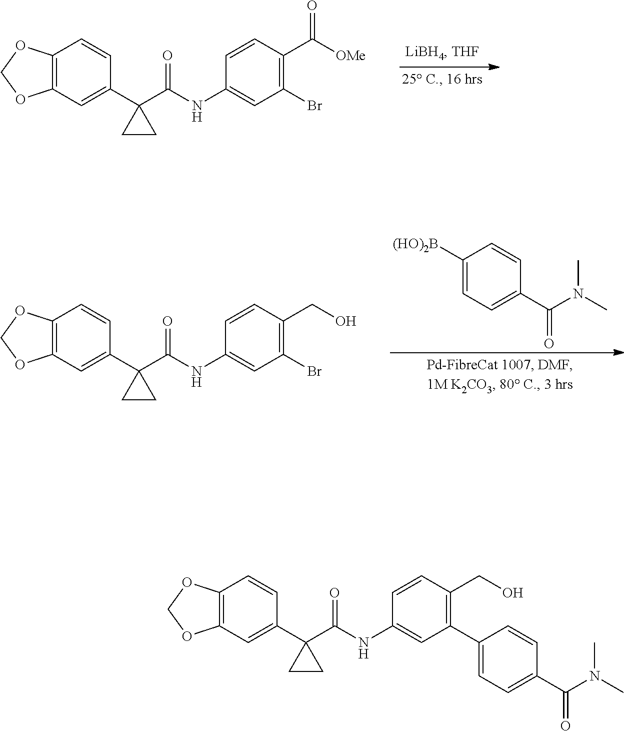 [SMILES: COC(=O)c1ccc(NC(=O)C2(c3ccc4c(c3)OCO4)CC2)cc1Br ; O=C(Nc1ccc(CO)c(Br)c1)C1(c2ccc3c(c2)OCO3)CC1 ; CN(C)C(=O)c1ccc(B(O)O)cc1 ; CN(C)C(=O)c1ccc(-c2cc(NC(=O)C3(c4ccc5c(c4)OCO5)CC3)ccc2CO)cc1]

### Step a: 1-(Benzo[d][1,3]dioxol-5-yl)-N-(3-bromo-4-(hydroxymethyl)phenyl)cyclopropanecarboxamide

Methyl 4-(1-(benzo[d][1,3]dioxol-5-yl)cyclopropanecarboxamido)-2-bromobenzoate (4.12 g, 9.9 mmol) was added to a solution of LiBH4 (429 mg, 19.8 mmol) in THF/ether/H2O (20/20/1 mL) and was allowed to stir at 25° C. After 16 hours, the reaction was quenched with H2O (10 mL). The reaction mixture was diluted with dichloromethane (25 mL) and was extracted with 1N HCl (30 mL×3) and brine (30 mL). The organic extracts were dried over Na2SO4 and evaporated. The crude product was purified by chromatography on silica gel (eluting with 0-100% ethyl acetate in hexanes) to afford 1-(benzo[d][1,3]dioxol-5-yl)-N-(3-bromo-4-(hydroxymethyl)phenyl)cyclopropanecarboxamide (2.84 g, 74%). ESI-MS m/z calc. 389.0. found 390.1 (M+1)+; retention time 2.91 minutes.

### Step b: 5′-(1-(Benzo[d][1,3]dioxol-5-yl)cyclopropanecarboxamido)-2′-(hydroxymethyl)-N,N-dimethylbiphenyl-4-carboxamide

1-(Benzo[d][1,3]dioxol-5-yl)-N-(3-bromo-4-(hydroxymethyl)-phenyl)cyclopropanecarboxamide (39 mg, 0.10 mmol), 4-(dimethylcarbamoyl)-phenylboronic acid (29 mg, 0.15 mmol), 1 M K2CO3 (0.3 mL, 0.3 mmol), Pd-FibreCat 1007 (8 mg, 0.1 mmol), and N,N-dimethylformamide (1 mL) were combined. The mixture was heated at 80° C. for 3 h. After cooling, the mixture was filtered and purified by reverse phase HPLC to yield 5′-(1-(benzo[d][1,3]dioxol-5-yl)cyclopropanecarboxamido)-2′-(hydroxymethyl)-N,N-dimethylbiphenyl-4-carboxamide (16 mg, 34%). ESI-MS m/z calc. 458.5. found 459.5 (M+1)+; Retention time 2.71 minutes.

### Preparation 14

### 5′-(1-(Benzo[d][1,3]dioxol-5-yl)cyclopropanecarboxamido)-2′-(ethoxymethyl)-N,N-dimethylbiphenyl-4-carboxamide

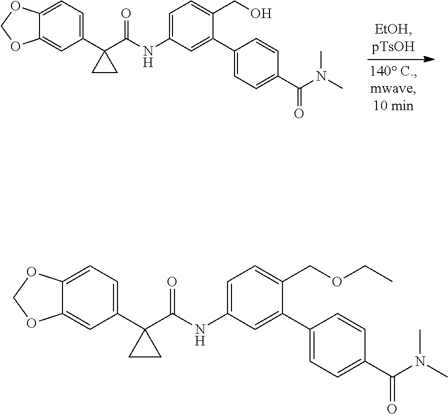 [SMILES: CN(C)C(=O)c1ccc(-c2cc(NC(=O)C3(c4ccc5c(c4)OCO5)CC3)ccc2CO)cc1 ; CCOCc1ccc(NC(=O)C2(c3ccc4c(c3)OCO4)CC2)cc1-c1ccc(C(=O)N(C)C)cc1]

5′-(1-(Benzo[d][1,3]dioxol-5-yl)cyclopropanecarboxamido)-2′-(hydroxymethyl)-N,N-dimethylbiphenyl-4-carboxamide (49 mg, 0.10 mmol) and para-toluenesulfonic acid (38 mg, 0.2 mmol) were dissolved in ethanol (1.0 mL) and irradiated in the microwave at 140° C. for 10 minutes. Volatiles were removed in vacuo and crude product was purified by reverse phase HPLC to afford the pure product (6.4 mg, 13%). ESI-MS m/z calc. 486.2. found 487.5 (M+1)+; retention time 3.17 minutes.

### Preparation 15

### 5′-(1-(Benzo[d][1,3]dioxol-5-yl)cyclopropane-carboxamido)-2′-(isopropoxymethyl)-N,N-dimethylbiphenyl-4-carboxamide

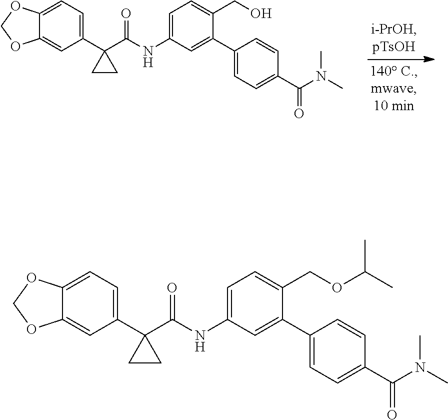 [SMILES: CN(C)C(=O)c1ccc(-c2cc(NC(=O)C3(c4ccc5c(c4)OCO5)CC3)ccc2CO)cc1 ; CC(C)OCc1ccc(NC(=O)C2(c3ccc4c(c3)OCO4)CC2)cc1-c1ccc(C(=O)N(C)C)cc1]

5′-(1-(Benzo[d][1,3]dioxol-5-yl)cyclopropanecarboxamido)-2′-(hydroxymethyl)-N,N-dimethylbiphenyl-4-carboxamide (46 mg, 0.10 mmol) and para-toluenesulfonic acid (38 mg, 0.2 mmol) were dissolved in isopropanol (1.0 mL) and irradiated in the microwave at 140° C. for 10 minutes. Volatiles were removed in vacuo and crude product was purified by reverse phase HPLC to afford the pure product (22 mg, 44%). ESI-MS m/z calc. 500.2. found 501.3 (M+1)+; retention time 3.30 minutes.

### Preparation 16

### 5′-(1-(Benzo[d][1,3]dioxol-5-yl)cyclopropanecarboxamido)-2′-(cyanomethyl)-N,N-dimethylbiphenyl-4-carboxamide

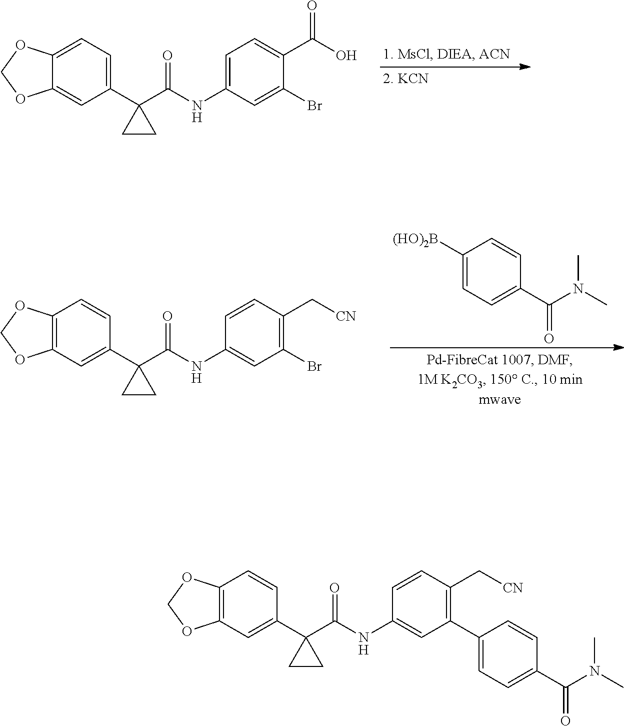 [SMILES: O=C(O)c1ccc(NC(=O)C2(c3ccc4c(c3)OCO4)CC2)cc1Br ; N#CCc1ccc(NC(=O)C2(c3ccc4c(c3)OCO4)CC2)cc1Br ; CN(C)C(=O)c1ccc(B(O)O)cc1 ; CN(C)C(=O)c1ccc(-c2cc(NC(=O)C3(c4ccc5c(c4)OCO5)CC3)ccc2CC#N)cc1]

### Step a: 1-(Benzo[d][1,3]dioxol-5-yl)-N-(3-bromo-4-(cyanomethyl)phenyl)cyclo-propane carboxamide

1-(Benzo[d][1,3]dioxol-5-yl)-N-(3-bromo-4-(hydroxymethyl)phenyl)cyclopropane-carboxamide (1.08 g, 2.78 mmol), methanesulfonyl chloride (0.24 mL, 3.1 mmol), and N,N-diisopropylethylamine (0.72 mL, 4.1 mmol) were dissolved in acetonitrile (27 mL) at 25° C. After complete dissolution, KCN (450 mg, 6.95 mmol) was added and the reaction was stirred for 14 d. The reaction was diluted with dichloromethane (25 mL) and washed with water (25 mL). The organic extracts were dried over Na2SO4 and evaporated. The crude product was purified by chromatography on silica gel (eluting with 0-100% ethyl acetate in hexanes) to afford 1-(benzo[d][1,3]dioxol-5-yl)-N-(3-bromo-4-(cyanomethyl)phenyl)cyclo-propane carboxamide (514 mg, 46%). ESI-MS m/z calc. 398.0. found 399.1 (M+1)+; retention time 3.24 minutes.

### Step b: 5′-(1-(Benzo[d][1,3]dioxol-5-yl)cyclopropanecarboxamido)-2′-(cyanomethyl)-N,N-dimethylbiphenyl-4-carboxamide

1-(Benzo[d][1,3]dioxol-5-yl)-N-(3-bromo-4-(cyanomethyl)phenyl)cyclopropane-carboxamide (40 mg, 0.10 mmol), 4-(dimethylcarbamoyl)phenylboronic acid (29 mg, 0.15 mmol), 1 M K2CO3 (0.2 mL, 0.2 mmol), Pd-FibreCat 1007 (8 mg, 0.1 mmol), and N,N-dimethylformamide (1 mL) were combined. The mixture was irradiated in the microwave at 150° C. for 10 minutes. Volatiles were removed in vacuo and crude product was purified by chromatography on silica gel (eluting with 0-100% ethyl acetate in hexanes) to afford 5′-(1-(benzo[d][1,3]dioxol-5-yl)cyclopropanecarboxamido)-2′-(cyanomethyl)-N,N-dimethylbiphenyl-4-carboxamide (9.1 mg, 20%). ESI-MS m/z calc. 467.2. found 468.5 (M+1)+; retention time 2.96 minutes.

### Preparation 17

### 2′-((1H-Tetrazol-5-yl)methyl)-5′-(1-(benzo[d][1,3]dioxol-5-yl)cyclopropane carboxamido)-N,N-dimethylbiphenyl-4-carboxamide

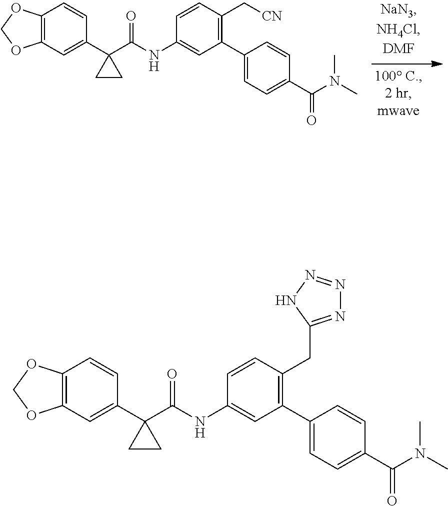 [SMILES: CN(C)C(=O)c1ccc(-c2cc(NC(=O)C3(c4ccc5c(c4)OCO5)CC3)ccc2CC#N)cc1 ; CN(C)C(=O)c1ccc(-c2cc(NC(=O)C3(c4ccc5c(c4)OCO5)CC3)ccc2Cc2nnn[nH]2)cc1]

5′-(1-(Benzo[d][1,3]dioxol-5-yl)cyclopropanecarboxamido)-2′-(cyanomethyl)-N,N-dimethylbiphenyl-4-carboxamide (32 mg, 0.070 mmol), sodium azide (55 mg, 0.84 mmol), and ammonium chloride (45 mg, 0.84 mmol) were dissolved in N,N-dimethylformamide (1.5 mL) and irradiated in the microwave at 100° C. for 2 hours. After cooling, the mixture was filtered and purified by reverse phase HPLC to yield 2′-((1H-tetrazol-5-yl)methyl)-5′-(1-(benzo[d][1,3]dioxol-5-yl)cyclopropane carboxamido)-N,N-dimethylbiphenyl-4-carboxamide (9.2 mg, 26%). ESI-MS m/z calc. 510.2. found 511.5 (M+1)+; Retention time 2.68 minutes.

### Preparation 18

### 2′-(2-Amino-2-oxoethyl)-5′-(1-(benzo[d][1,3]dioxol-5-yl)cyclopropanecarboxamido)-N,N-dimethylbiphenyl-4-carboxamide

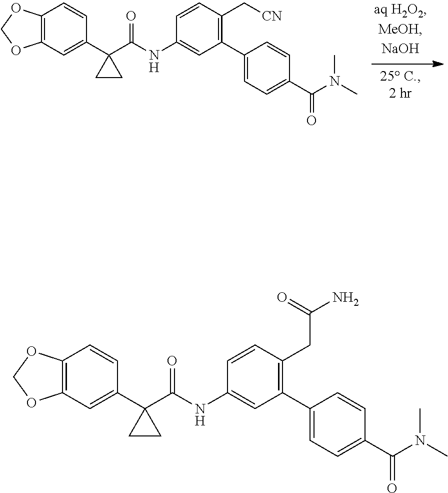 [SMILES: CN(C)C(=O)c1ccc(-c2cc(NC(=O)C3(c4ccc5c(c4)OCO5)CC3)ccc2CC#N)cc1 ; CN(C)C(=O)c1ccc(-c2cc(NC(=O)C3(c4ccc5c(c4)OCO5)CC3)ccc2CC(N)=O)cc1]

5′-(1-(Benzo[d][1,3]dioxol-5-yl)cyclopropanecarboxamido)-2′-(cyanomethyl)-N,N-dimethylbiphenyl-4-carboxamide (58 mg, 0.12 mmol), H2O2 (30 wt % solution in water, 36 μL, 1.2 mmol), and NaOH (10 wt % in water, 0.15 mL, 0.42 mmol) were dissolved in MeOH (1.2 mL) and stirred at 25° C. for 2 hours. The reaction was filtered and purified by reverse phase HPLC to yield 2′-(2-amino-2-oxoethyl)-5′-(1-(benzo[d][1,3]dioxol-5-yl)cyclopropanecarboxamido)-N,N-dimethylbiphenyl-4-carboxamide (14 mg, 23%). ESI-MS m/z calc. 485.2. found 486.5 (M+1)+; Retention time 2.54 minutes.

### Preparation 19

### N-(4′-(Aminomethyl)-6-methylbiphenyl-3-yl)-1-(benzo[d][1,3]dioxol-5-yl)cyclopropanecarboxamide

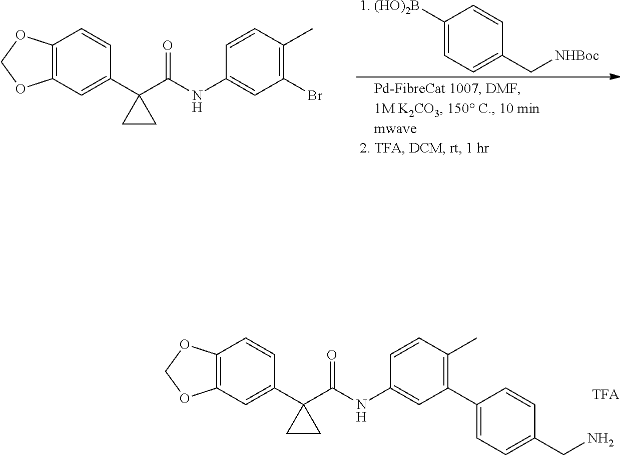

1-(Benzo[d][1,3]dioxol-5-yl)-N-(3-bromo-4-methylphenyl)cyclopropanecarboxamide (37 mg, 0.10 mmol), 4-((tert-butoxycarbonylamino)methyl)phenylboronic acid (37 mg, 0.15 mmol), 1 M K2CO3 (0.2 mL, 0.2 mmol), Pd-FibreCat 1007 (8 mg, 0.1 mmol), and N,N-dimethylformamide (1 mL) were combined. The mixture was irradiated in the microwave at 150° C. for 10 minutes. The reaction was filtered and purified by reverse phase HPLC. The obtained material was dissolved in dichloromethane (2 mL) containing trifluoroacetic acid (2 mL) and stirred at 25° C. for 1 hour. The reaction was filtered and purified by reverse phase HPLC to yield N-(4′-(aminomethyl)-6-methylbiphenyl-3-yl)-1-(benzo[d][1,3]dioxol-5-yl)cyclopropanecarboxamide as the TFA salt (8.1 mg, 20%). ESI-MS m/z calc. 400.2. found 401.5 (M+1)+; retention time 2.55 minutes.

### Preparation 20

### 1-(Benzo[d][1,3]dioxol-5-yl)-N-(6-methyl-4′-(propionamidomethyl)biphenyl-3-yl)cyclopropanecarboxamide

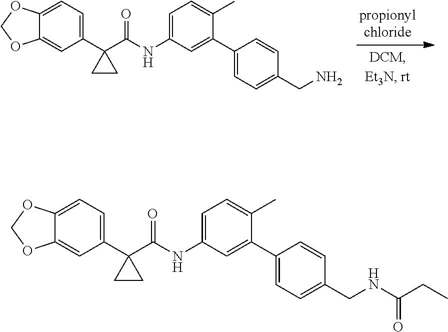 [SMILES: Cc1ccc(NC(=O)C2(c3ccc4c(c3)OCO4)CC2)cc1-c1ccc(CN)cc1 ; CCC(=O)NCc1ccc(-c2cc(NC(=O)C3(c4ccc5c(c4)OCO5)CC3)ccc2C)cc1]

N-(4′-(Aminomethyl)-6-methylbiphenyl-3-yl)-1-(benzo[d][1,3]dioxol-5-yl)cyclopropanecarboxamide (40 mg, 0.10 mmol), propionyl chloride (8.7 μL, 0.10 mmol) and Et3N (28 μL, 0.20 mmol) were dissolved in dichloromethane (1.0 mL) and allowed to stir at 25° C. for 3 hours. Volatiles were removed in vacuo and crude product was purified by reverse phase HPLC to afford 1-(benzo[d][1,3]dioxol-5-yl)-N-(6-methyl-4′-(propionamidomethyl)biphenyl-3-yl)cyclopropanecarboxamide (13 mg, 28%). ESI-MS m/z calc. 456.5. found 457.5 (M+1)+; retention time 3.22 minutes.

### Preparation 21

### 1-(Benzo[d][1,3]dioxol-5-yl)-N-(6-methyl-4′-(propylsulfonamidomethyl)biphenyl-3-yl)cyclopropanecarboxamide

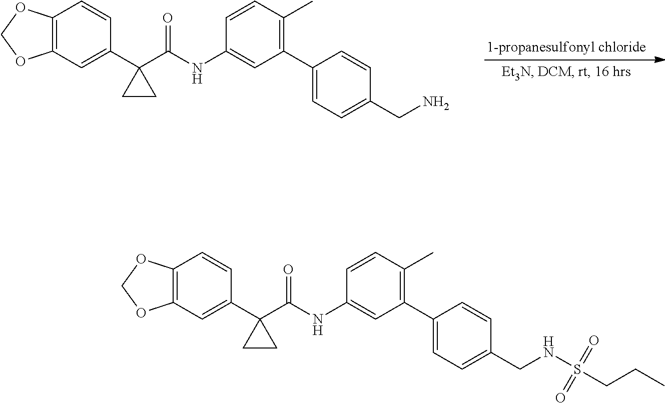 [SMILES: Cc1ccc(NC(=O)C2(c3ccc4c(c3)OCO4)CC2)cc1-c1ccc(CN)cc1 ; CCCS(=O)(=O)NCc1ccc(-c2cc(NC(=O)C3(c4ccc5c(c4)OCO5)CC3)ccc2C)cc1]

N-(4′-(Aminomethyl)-6-methylbiphenyl-3-yl)-1-(benzo[d][1,3]dioxol-5-yl)cyclopropanecarboxamide (40 mg, 0.10 mmol), 1-propanesulfonyl chloride (11 μL, 0.10 mmol) and Et3N (28 μL, 0.20 mmol) were dissolved in dichloromethane (1.0 mL) and allowed to stir at 25° C. for 16 hours. Volatiles were removed in vacuo and crude product was purified by reverse phase HPLC to afford 1-(benzo[d][1,3]dioxol-5-yl)-N-(6-methyl-4′-(propylsulfonamidomethyl)biphenyl-3-yl)cyclopropanecarboxamide (5.3 mg, 10%). ESI-MS m/z calc. 506.6. found 507.3 (M+1)+; retention time 3.48 minutes.

### Preparation 22

### 1-(Benzo[d][1,3]dioxol-5-yl)-N-(6-methyl-4′-((propylamino)methyl)biphenyl-3-yl)cyclopropanecarboxamide

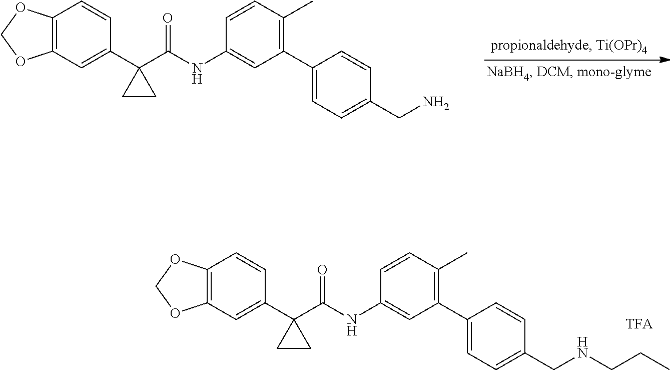 [SMILES: Cc1ccc(NC(=O)C2(c3ccc4c(c3)OCO4)CC2)cc1-c1ccc(CN)cc1 ; CCCNCc1ccc(-c2cc(NC(=O)C3(c4ccc5c(c4)OCO5)CC3)ccc2C)cc1]

N-(4′-(Aminomethyl)-6-methylbiphenyl-3-yl)-1-(benzo[d][1,3]dioxol-5-yl)cyclopropanecarboxamide (40 mg, 0.10 mmol), propionaldehyde (5.1 μL, 0.10 mmol) and Ti(OPr)4 (82 μL, 0.30 mmol) were dissolved in dichloromethane (1.0 mL) and mono-glyme (1.0 mL). The mixture was allowed to stir at 25° C. for 16 hours. NaBH4 (5.7 mg, 0.15 mmol) was added and the reaction was stirred for an additional 1 h. The reaction was diluted to 5 mL with dichloromethane before water (5 mL) was added. The reaction was filtered through celite to remove the titanium salts and the layers separated. The organic extracts were dried over Na2SO4 and evaporated. The crude product was purified by reverse phase HPLC to afford 1-(benzo[d][1,3]dioxol-5-yl)-N-(6-methyl-4′-((propylamino)methyl)biphenyl-3-yl)cyclopropanecarboxamide (7.8 mg, 14%). ESI-MS m/z calc. 442.6. found 443.5 (M+1)+; retention time 2.54 minutes.

### Preparation 23

### 1-(Benzo[d][1,3]dioxol-5-yl)-N-(4′-((isopentylamino)methyl)-6-methylbiphenyl-3-yl)cyclopropanecarboxamide

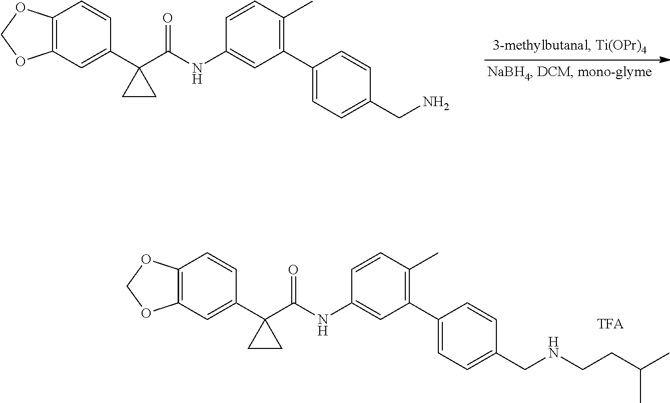 [SMILES: Cc1ccc(NC(=O)C2(c3ccc4c(c3)OCO4)CC2)cc1-c1ccc(CN)cc1 ; Cc1ccc(NC(=O)C2(c3ccc4c(c3)OCO4)CC2)cc1-c1ccc(CNCCC(C)C)cc1]

N-(4′-(Aminomethyl)-6-methylbiphenyl-3-yl)-1-(benzo[d][1,3]dioxol-5-yl)cyclopropanecarboxamide (40 mg, 0.10 mmol), 3-methylbutanal (8.6 mg, 0.10 mmol) and Ti(OPr)4 (82 μL, 0.30 mmol) were dissolved in dichloromethane (1.0 mL) and mono-glyme (1.0 mL) and allowed to stir at 25° C. for 16 hours. NaBH4 (5.7 mg, 0.15 mmol) was added and the reaction was stirred for an additional 1 h. The reaction was diluted to 5 mL with dichloromethane before water (5 mL) was added. The reaction was filtered through celite to remove the titanium salts and the layers separated. The organic extracts were dried over Na2SO4 and evaporated. The crude product was purified by reverse phase HPLC to afford 1-(benzo[d][1,3]dioxol-5-yl)-N-(4′-((isopentylamino)methyl)-6-methylbiphenyl-3-yl)cyclopropanecarboxamide (5.7 mg, 10%). ESI-MS m/z calc. 470.3. found 471.5 (M+1)+; retention time 2.76 minutes.

### Preparation 24

### 1-(Benzo[d][1,3]dioxol-5-yl)-N-(4′-(hydroxymethyl)-6-methylbiphenyl-3-yl)cyclopropanecarboxamide

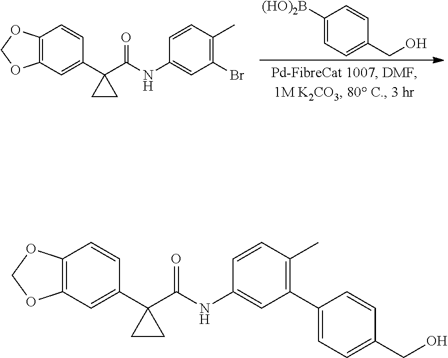 [SMILES: Cc1ccc(NC(=O)C2(c3ccc4c(c3)OCO4)CC2)cc1Br ; OCc1ccc(B(O)O)cc1 ; Cc1ccc(NC(=O)C2(c3ccc4c(c3)OCO4)CC2)cc1-c1ccc(CO)cc1]

1-(Benzo[d][1,3]dioxol-5-yl)-N-(3-bromo-4-methylphenyl)cyclopropanecarboxamide (3.0 g, 8.1 mmol), 4-(hydroxymethyl)phenylboronic acid (1.5 g, 9.7 mmol), 1 M K2CO3 (16 mL, 16 mmol), Pd-FibreCat 1007 (640 mg), and N,N-dimethylformamide (80 mL) were combined. The mixture was heated at 80° C. for 3 h. The volatiles were removed in vacuo and residue was redissolved in dichloromethane (100 mL). The organics were washed with 1N HCl (100 mL×2), then dried over Na2SO4 and evaporated. The crude product was purified by chromatography on silica gel to afford 1-(benzo[d][1,3]dioxol-5-yl)-N-(4′-(hydroxymethyl)-6-methylbiphenyl-3-yl)cyclopropanecarboxamide (1.9 g, 59%). ESI-MS m/z calc. 401.5. found 402.5 (M+1)+; retention time 3.18 minutes.

### Preparation 25

### 1-(Benzo[d][1,3]dioxol-5-yl)-N-(4′-(methoxymethyl)-6-methylbiphenyl-3-yl)cyclopropanecarboxamide

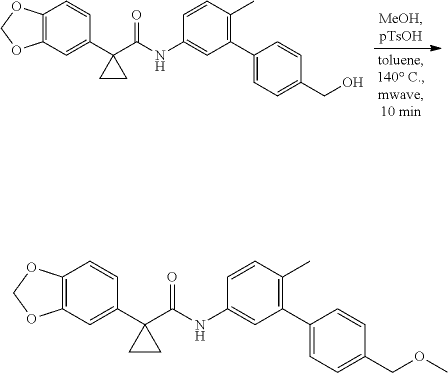 [SMILES: Cc1ccc(NC(=O)C2(c3ccc4c(c3)OCO4)CC2)cc1-c1ccc(CO)cc1 ; COCc1ccc(-c2cc(NC(=O)C3(c4ccc5c(c4)OCO5)CC3)ccc2C)cc1]

1-(Benzo[d][1,3]dioxol-5-yl)-N-(4′-(hydroxymethyl)-6-methylbiphenyl-3-yl)cyclopropanecarboxamide (40 mg, 0.10 mmol), para-toluenesulfonic acid (24 mg, 0.13 mmol) and MeOH (53 μL, 1.3 mmol) were dissolved in toluene (2.0 mL) and irradiated in the microwave at 140° C. for 10 minutes. Volatiles were removed in vacuo and crude product was purified by reverse phase HPLC to afford 1-(benzo[d][1,3]dioxol-5-yl)-N-(4′-(methoxymethyl)-6-methylbiphenyl-3-yl)cyclopropanecarboxamide (9.6 mg, 23%). ESI-MS m/z calc. 415.5. found 416.5 (M+1)+; retention time 3.68 minutes.

### Preparation 26

### 1-(Benzo[d][1,3]dioxol-5-yl)-N-(6-methyl-4′-((methylamino)methyl)biphenyl-3-yl)cyclopropanecarboxamide

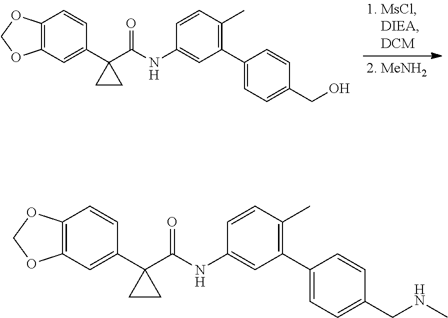 [SMILES: Cc1ccc(NC(=O)C2(c3ccc4c(c3)OCO4)CC2)cc1-c1ccc(CO)cc1 ; CNCc1ccc(-c2cc(NC(=O)C3(c4ccc5c(c4)OCO5)CC3)ccc2C)cc1]

1-(Benzo[d][1,3]dioxol-5-yl)-N-(4′-(hydroxymethyl)-6-methylbiphenyl-3-yl)cyclopropanecarboxamide (610 mg, 1.52 mmol), methanesulfonyl chloride (0.13 mL, 1.7 mmol), and N,N-diisopropylethylamine (0.79 mL, 4.6 mmol) were dissolved in dichloromethane (10 mL) at 25° C. The reaction was stirred for 10 minutes before a 2.0 M solution of MeNH2 in THF (15 mL, 30 mmol) was added. The mixture was stirred for 30 minutes at ambient temperature before it was extracted with 1N HCl (20 mL×2) and saturated NaHCO3 (20 mL×2). The organic extracts were dried over Na2SO4 and evaporated. The crude product was purified by chromatography on silica gel (eluting with 0-20% methanol in dichloromethane) to afford 1-(Benzo[d][1,3]dioxol-5-yl)-N-(6-methyl-4′-((methylamino)methyl)biphenyl-3-yl)cyclopropanecarboxamide (379 mg, 60%). ESI-MS m/z calc. 414.5. found 415.5 (M+1)+; retention time 2.44 minutes.

### Preparation 27

### 1-(Benzo[d][1,3]dioxol-5-yl)-N-(6-methyl-4′-((N-methylpivalamido)methyl)biphenyl-3-yl)cyclopropanecarboxamide

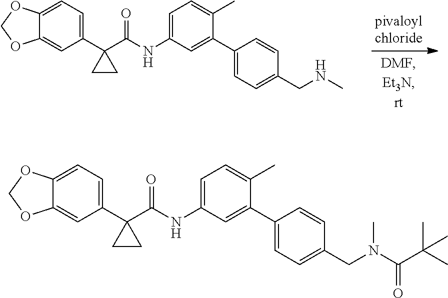 [SMILES: CNCc1ccc(-c2cc(NC(=O)C3(c4ccc5c(c4)OCO5)CC3)ccc2C)cc1 ; Cc1ccc(NC(=O)C2(c3ccc4c(c3)OCO4)CC2)cc1-c1ccc(CN(C)C(=O)C(C)(C)C)cc1]

1-(Benzo[d][1,3]dioxol-5-yl)-N-(6-methyl-4′-((methylamino)methyl)biphenyl-3-yl)cyclopropanecarboxamide (30 mg, 0.070 mmol), pivaloyl chloride (12.3 μL, 0.090 mmol) and Et3N (20 μL, 0.14 mmol) were dissolved in N,N-dimethylformamide (1.0 mL) and allowed to stir at 25° C. for 3 hours. The crude reaction was purified by reverse phase HPLC to afford 1-(benzo[d][1,3]dioxol-5-yl)-N-(6-methyl-4′-((N-methylpivalamido)methyl)biphenyl-3-yl)cyclopropanecarboxamide (15 mg, 30%). ESI-MS m/z calc. 498.3. found 499.3 (M+1)+; retention time 3.75 minutes.

### Preparation 28

### 1-(Benzo[d][1,3]dioxol-5-yl)-N-(6-methyl-4′-((N-methylmethylsulfonamido) methyl)biphenyl-3-yl)cyclopropanecarboxamide

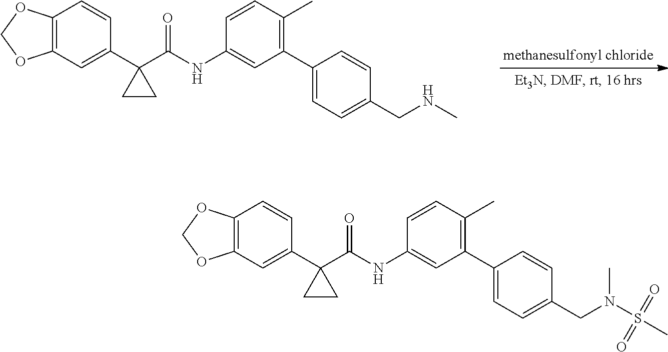 [SMILES: CNCc1ccc(-c2cc(NC(=O)C3(c4ccc5c(c4)OCO5)CC3)ccc2C)cc1 ; Cc1ccc(NC(=O)C2(c3ccc4c(c3)OCO4)CC2)cc1-c1ccc(CN(C)S(C)(=O)=O)cc1]

1-(Benzo[d][1,3]dioxol-5-yl)-N-(6-methyl-4′-((methylamino)-methyl)biphenyl-3-yl)cyclopropane carboxamide (30 mg, 0.070 mmol), methanesulfonyl chloride (7.8 μL, 0.14 mmol) and Et3N (30 μL, 0.22 mmol) were dissolved in N,N-dimethylformamide (1.0 mL) and allowed to stir at 25° C. for 16 hours. The crude reaction was purified by reverse phase HPLC to afford 1-(benzo[d][1,3]dioxol-5-yl)-N-(6-methyl-4′-((N-methylmethylsulfonamido) methyl)biphenyl-3-yl)cyclopropanecarboxamide (22 mg, 64%). ESI-MS m/z calc. 492.2. found 493.3 (M+1)+; retention time 3.45 minutes.

### Preparation 29

### 1-(Benzo[d][1,3]dioxol-5-yl)-N-(4′-((isobutyl(methyl)amino)-methyl)-6-methylbiphenyl-3-yl)cyclopropanecarboxamide

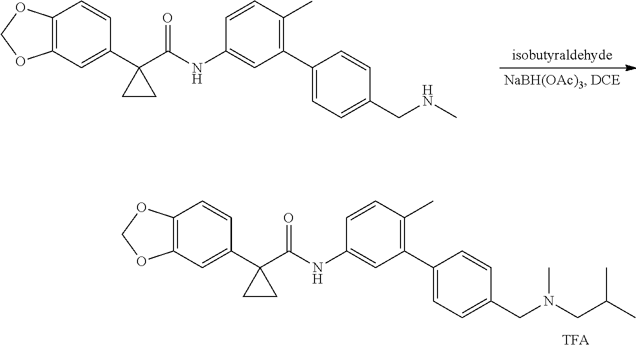 [SMILES: CNCc1ccc(-c2cc(NC(=O)C3(c4ccc5c(c4)OCO5)CC3)ccc2C)cc1 ; Cc1ccc(NC(=O)C2(c3ccc4c(c3)OCO4)CC2)cc1-c1ccc(CN(C)CC(C)C)cc1]

1-(Benzo[d][1,3]dioxol-5-yl)-N-(6-methyl-4′-((methylamino)methyl)biphenyl-3-yl)cyclopropanecarboxamide (49 mg, 0.12 mmol), isobutyraldehyde (11 μL, 0.12 mmol) and NaBH(OAc)3 (76 mg, 0.36 mmol) were dissolved in dichloroethane (2.0 mL) and heated at 70° C. for 16 hours. The reaction was quenched with MeOH (0.5 mL) and 1N HCl (0.5 mL). The volatiles were removed in vacuo and the crude product was purified by reverse phase HPLC to afford 1-(benzo[d][1,3]dioxol-5-yl)-N-(4′-((isobutyl(methyl)amino)-methyl)-6-methylbiphenyl-3-yl)cyclopropanecarboxamide as the TFA salt (5.0 mg, 9%). ESI-MS m/z calc. 470.3. found 471.3 (M+1)+; retention time 2.64 minutes.

The following compounds were prepared using procedures 20-23 and 27-29 above: 6, 14, 24, 26, 70, 79, 84, 96, 114, 122, 159, 200, 206, 214, 223, 248, 284-5, 348, 355, 382, 389, 391, 447, 471, 505, 511, 524, 529-30, 534, 551, 562, 661, 682, 709, 783, 786, 801, 809, 828, 844, 846, 877, 937, 947, 1012, 1049, 1089.

### Preparation 30

### 1-(Benzo[d][1,3]dioxol-5-yl)-N-(4-(2-methylthiazol-4-yl)phenyl)cyclopropane-carboxamide

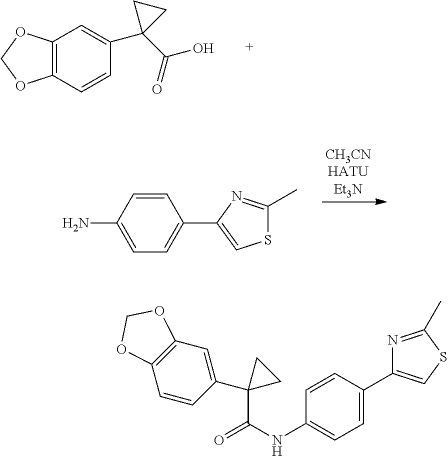 [SMILES: O=C(O)C1(c2ccc3c(c2)OCO3)CC1 ; Cc1nc(-c2ccc(N)cc2)cs1 ; Cc1nc(-c2ccc(NC(=O)C3(c4ccc5c(c4)OCO5)CC3)cc2)cs1]

4-(2-Methylthiazol-4-yl)aniline (19 mg, 0.10 mmol) and 1-(benzo[d][1,3]dioxol-5-yl)cyclopropanecarboxylic acid (20.6 mg, 0.100 mmol) were dissolved in acetonitrile (1.0 mL) containing triethylamine (42 μL, 0.30 mmol). O-(7-Azabenzotriazol-1-yl)-N,N,N′,N′-tetramethyluronium hexafluorophosphate (42 mg, 0.11 mmol) was added to the mixture and the resulting solution was allowed to stir for 16 hours. The crude product was purified by reverse-phase preparative liquid chromatography to yield 1-(benzo[d][1,3]dioxol-5-yl)-N-(4-(2-methylthiazol-4-yl)phenyl)cyclopropane-carboxamide. ESI-MS m/z calc. 378.1. found; 379.1 (M+1)+; Retention time 2.72 minutes. 1H NMR (400 MHz, DMSO-d6) δ 1.04-1.10 (m, 2H), 1.40-1.44 (m, 2H), 2.70 (s, 3H), 6.03 (s, 2H), 6.88-6.96 (m, 2H), 7.01 (d, J=1.4 Hz, 1H), 7.57-7.61 (m, 2H), 7.81-7.84 (m, 3H), 8.87 (s, 1H).

### Preparation 31

### 1-Benzo[1,3]dioxol-5-yl-N-[3-[4-(methylsulfamoyl)phenyl]phenyl]-cyclopropane-1-carboxamide

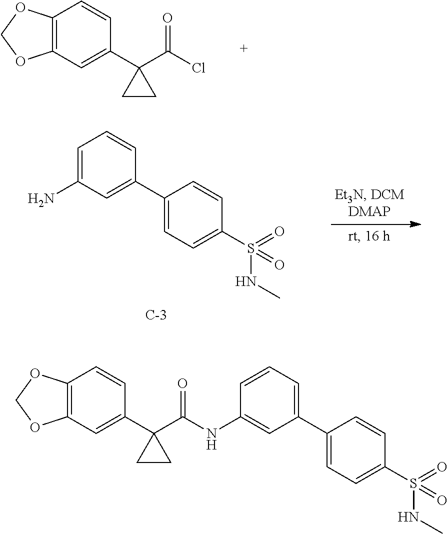

To a solution of 1-benzo[1,3]dioxol-5-yl-cyclopropanecarbonyl chloride (0.97 mmol) in CH2Cl2 (3 mL) at ambient temperature was added a solution of 3′-amino-N-methylbiphenyl-4-sulfonamide (0.25 g, 0.97 mmol), Et3N (0.68 mL, 4.9 mmol), DMAP (0.050 g, 0.058 mmol), and CH2Cl2 (1 mL) dropwise. The mixture was allowed to stir for 16 h before it was diluted with CH2Cl2 (50 mL). The solution was washed with 1N HCl (2×25 mL), sat. aq. NaHCO3 (2×25 mL), then brine (25 mL). The organics were dried over Na2SO4, filtered, and concentrated in vacuo. The residue was purified by column chromatography (5-25% EtOAc/hexanes) to provide 1-benzo[1,3]dioxol-5-yl-N-[3-[4-(methylsulfamoyl)phenyl]phenyl]-cyclopropane-1-carboxamide as a white solid. ESI-MS m/z calc. 450.5. found 451.3 (M+1)+. Retention time of 3.13 minutes.

The following compounds were prepared using procedures 30 and 31 above: 4-5, 27, 35, 39, 51, 55, 75, 81, 90, 97-8, 101, 110, 132, 146, 155, 166, 186, 208, 211, 218, 230, 239, 245, 247, 258, 261, 283, 292, 308, 334, 339, 352, 356, 379, 405, 411, 433, 462, 477, 504, 514, 526, 536, 554, 563, 573, 590-2, 612, 619, 623, 627, 637, 648, 653, 660, 668-9, 692, 728, 740, 747, 748, 782, 814, 826-7, 834-6, 845, 916, 931-2, 938, 944, 950, 969, 975, 996, 1004, 1007, 1009, 1033, 1064, 1084-5, 1088, 1097, 1102, 1127, 1151, 1157, 1159, 1162, 1186, 1193.

### Preparation 32

### 4-[5-(1-Benzo[1,3]dioxol-5-ylcyclopropyl)carbonylamino-2-methyl-phenyl]benzoic acid

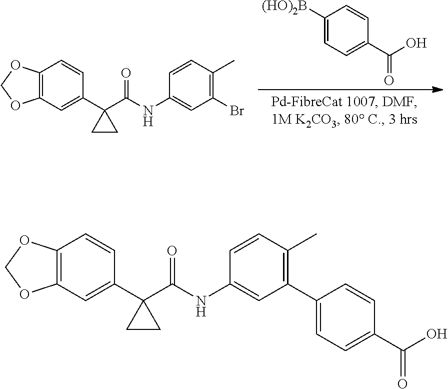 [SMILES: Cc1ccc(NC(=O)C2(c3ccc4c(c3)OCO4)CC2)cc1Br ; O=C(O)c1ccc(B(O)O)cc1 ; Cc1ccc(NC(=O)C2(c3ccc4c(c3)OCO4)CC2)cc1-c1ccc(C(=O)O)cc1]

1-(Benzo[d][1,3]dioxol-5-yl)-N-(3-bromo-4-methylphenyl)cyclopropanecarbox-amide (B-8) (5.1 g, 14 mmol), 4-boronobenzoic acid (3.4 g, 20 mmol), 1 M K2CO3 (54 mL, 54 mmol), Pd-FibreCat 1007 (810 mg, 1.35 mmol), and DMF (135 mL) were combined. The mixture was heated at 80° C. for 3 h. After cooling, the mixture was filtered and DMF was removed in vacuo. The residue was partitioned between dichloromethane (250 mL) and 1N HCl (250 mL). The organics were separated, washed with saturated NaCl solution (250 mL), and dried over Na2SO4. Evaporation of organics yielded 4-[5-(1-benzo[1,3]dioxol-5-ylcyclopropyl)carbonylamino-2-methyl-phenyl]benzoic acid (5.5 g, 98%). ESI-MS m/z calc. 415.1. found 416.5 (M+1)+; Retention time 3.19 minutes. 1H NMR (400 MHz, DMSO-d6) δ 13.06 (s, 1H), 8.83 (s, 1H), 8.06-8.04 (m, 2H), 7.58-7.56 (m, 1H), 7.50-7.48 (m, 3H), 7.27-7.24 (m, 1H), 7.05-7.04 (m, 1H), 6.98-6.94 (m, 2H), 6.07 (s, 2H), 2.22 (s, 3H), 1.46-1.44 (m, 2H), 1.12-1.09 (m, 2H).

### Preparation 33

### 5′-(1-(Benzo[d][1,3]dioxol-5-yl)cyclopropanecarboxamido)-2′-methyl-N-(2-(pyridin-2-yl)ethyl)biphenyl-4-carboxamide

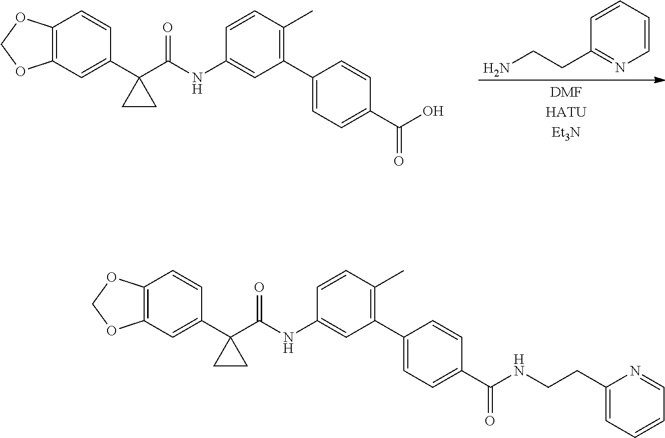 [SMILES: Cc1ccc(NC(=O)C2(c3ccc4c(c3)OCO4)CC2)cc1-c1ccc(C(=O)O)cc1 ; NCCc1ccccn1 ; Cc1ccc(NC(=O)C2(c3ccc4c(c3)OCO4)CC2)cc1-c1ccc(C(=O)NCCc2ccccn2)cc1]

2-(Pyridin-2-yl)ethanamine (12 mg, 0.10 mmol) and 5′-(1-(benzo[d][1,3]dioxol-5-yl)cyclopropanecarboxamido)-2′-methylbiphenyl-4-carboxylic acid (42 mg, 0.10 mmol) were dissolved in N,N-dimethylformamide (1.0 mL) containing triethylamine (28 μL, 0.20 mmol). O-(7-Azabenzotriazol-1-yl)-N,N,N′N-tetramethyluronium hexafluorophosphate (42 mg, 0.11 mmol) was added to the mixture and the resulting solution was allowed to stir for 1 hour at ambient temperature. The crude product was purified by reverse-phase preparative liquid chromatography to yield 5′-(1-(benzo[d][1,3]dioxol-5-yl)cyclopropanecarboxamido)-2′-methyl-N-(2-(pyridin-2-yl)ethyl)biphenyl-4-carboxamide as the trifluoroacetic acid salt (43 mg, 67%). ESI-MS m/z calc. 519.2. found 520.5 (M+1)+; Retention time 2.41 minutes. 1H NMR (400 MHz, DMSO-d6) δ 8.77 (s, 1H), 8.75-8.74 (m, 1H), 8.68-8.65 (m, 1H), 8.23 (m, 1H), 7.83-7.82 (m, 2H), 7.75-7.68 (m, 2H), 7.48-7.37 (m, 4H), 7.20-7.18 (m, 1H), 6.99-6.98 (m, 1H), 6.90-6.89 (m, 2H), 6.01 (s, 2H), 3.72-3.67 (m, 2H), 3.20-3.17 (m, 2H), 2.15 (s, 3H), 1.40-1.37 (m, 2H), 1.06-1.03 (m, 2H).

The following compounds were prepared using procedure 33 above: 32, 78, 118, 134, 156, 171, 188, 237, 279, 291, 297, 309, 319, 338, 341, 362, 373, 376, 393, 406-7, 410, 448, 452-3, 474, 482, 494, 508, 577, 580, 593-4, 622, 629, 638, 651, 663-4, 681, 698, 704, 707, 710, 736-7, 739, 775, 806, 810, 825, 842, 853, 866, 871, 900, 905-7, 926, 935, 941, 966, 971, 973, 978-9, 1046, 1048, 1066, 1077, 1079, 1083, 1141, 1150, 1155-6, 1163, 1180, 1185, 1187, 1198, 1201.

### Preparation 34

### 4-[5-(1-Benzo[1,3]dioxol-5-ylcyclopropyl)carbonylamino-2-methyl-phenyl]-N,N-dimethyl-benzamide

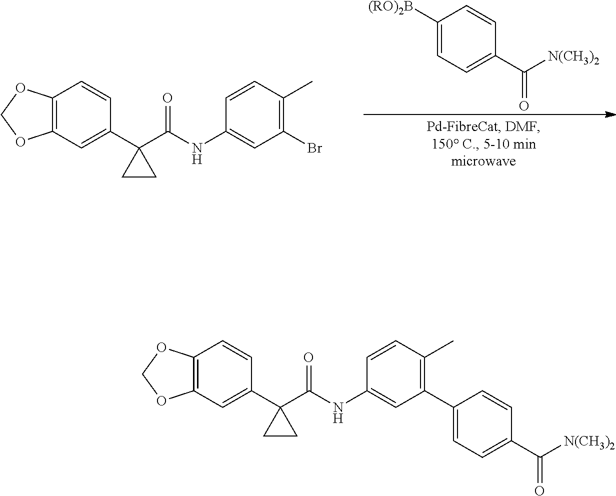

1-(Benzo[d][1,3]dioxol-5-yl)-N-(3-bromo-4-methylphenyl)cyclopropanecarbox-amide (0.10 mmol), N,N-dimethyl-4-(4,4,5,5-tetramethyl-1,3,2-dioxaborolan-2-yl)benzamide (0.11 mmol), K2CO3 (240 μL, 1M), Pd-FibreCat (7 mg), and DMF (1 mL) were combined. The mixture was heated at 150° C. for 5 min (5 min ramp time) in a microwave reactor. After cooling, the mixture was filtered and purified by prep-HPLC to provide 4-[5-(1-benzo[1,3]dioxol-5-ylcyclopropyl)carbonylamino-2-methyl-pheny]-N,N-dimethyl-benzamide. ESI-MS m/z calc. 442.2. found 443.5 (M+1)+; Retention time 3.12 minutes. 1H NMR (400 MHz, DMSO-d6) δ 1.02-1.08 (m, 2H), 1.37-1.44 (m, 2H), 2.17 (s, 3H), 2.96 (s, 3H), 3.00 (s, 3H), 6.01 (s, 2H), 6.87-6.93 (m, 2H), 6.98 (d, J=1.3 Hz, 1H), 7.19 (d, J=8.4 Hz, 1H), 7.34-7.37 (m, 2H), 7.40-7.52 (m, 4H), 8.75 (s, 1H).

### Preparation 35

### 5′-(1-(Benzo[d][1,3]dioxol-5-yl)cyclopropanecarboxamido)-2′-(isopropoxymethyl)-N,N-dimethylbiphenyl-4-carboxamide

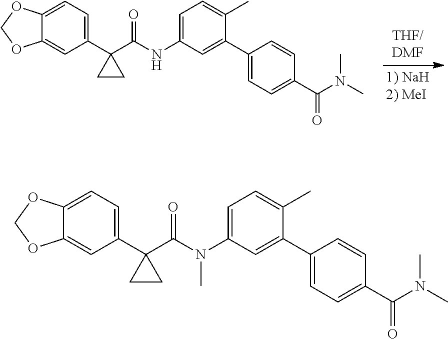 [SMILES: Cc1ccc(NC(=O)C2(c3ccc4c(c3)OCO4)CC2)cc1-c1ccc(C(=O)N(C)C)cc1 ; Cc1ccc(N(C)C(=O)C2(c3ccc4c(c3)OCO4)CC2)cc1-c1ccc(C(=O)N(C)C)cc1]

Sodium hydride (2.2 mg, 0.055 mmol, 60% by weight dispersion in oil) was slowly added to a stirred solution of 5′-(1-(benzo[d][1,3]dioxol-5-yl)cyclopropanecarboxamido)-N,N,2′-trimethylbiphenyl-4-carboxamide (21 mg, 0.048 mmol) in a mixture of 0.90 mL of anhydrous tetrahydrofuran (THF) and 0.10 mL of anhydrous N,N-dimethylformamide (DMF). The resulting suspension was allowed to stir for 3 minutes before iodomethane (0.0048 mL, 0.072 mmol) was added to the reaction mixture. An additional aliquot of sodium hydride and iodomethane were required to consume all of the starting material which was monitored by LCMS. The crude reaction product was evaporated to dryness, redissolved in a minimum of DMF and purified by preparative LCMS chromatography to yield 5′-(1-(benzo[d][1,3]dioxol-5-yl)cyclopropanecarboxamido)-2′-(isopropoxymethyl)-N,N-dimethylbiphenyl-4-carboxamide (9.1 mg, 42%) ESI-MS m/z calc. 456.2. found 457.5 (M+1)+. Retention time of 2.94 minutes. 1H NMR (400 MHz, CD3CN) δ 0.91-0.93 (m, 2H), 1.41-1.45 (m, 2H), 2.23 (s, 3H), 3.00 (s, 3H), 3.07 (s, 3H), 3.20 (s, 3H), 5.81 (s, 2H), 6.29-6.36 (m, 2H), 6.56 (d, J=8.0 Hz, 1H), 6.69 (s, 1H), 6.92 (dd, J=1.6, 7.9 Hz, 1H), 7.17 (d, J=8.1 Hz, 1H), 7.28 (d, J=8.1 Hz, 2H), 7.46 (dd, J=1.8, 6.4 Hz, 2H).

### Preparation 36

### (S)-1-(5′-(1-(Benzo[d][1,3]dioxol-5-yl)cyclopropanecarboxamido)-2′-methylbiphenyl-4-ylsulfonyl)pyrrolidine-2-carboxylic acid

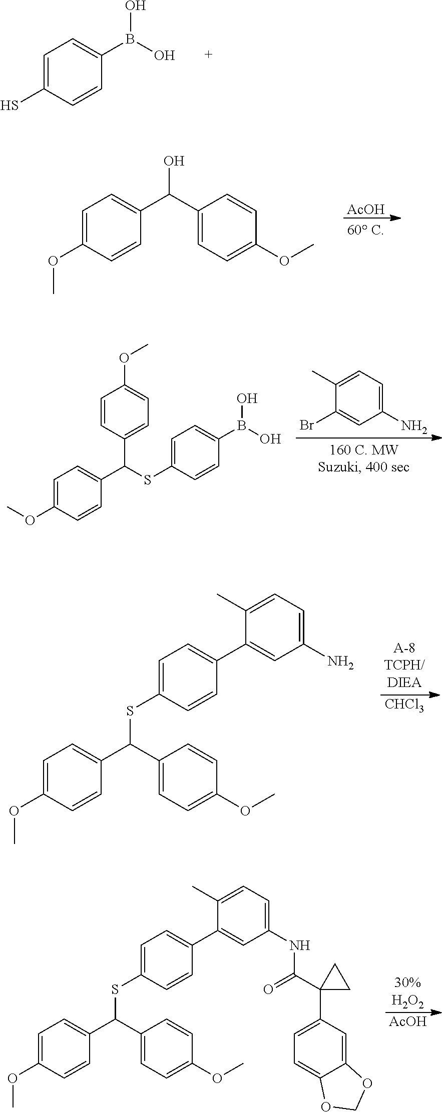

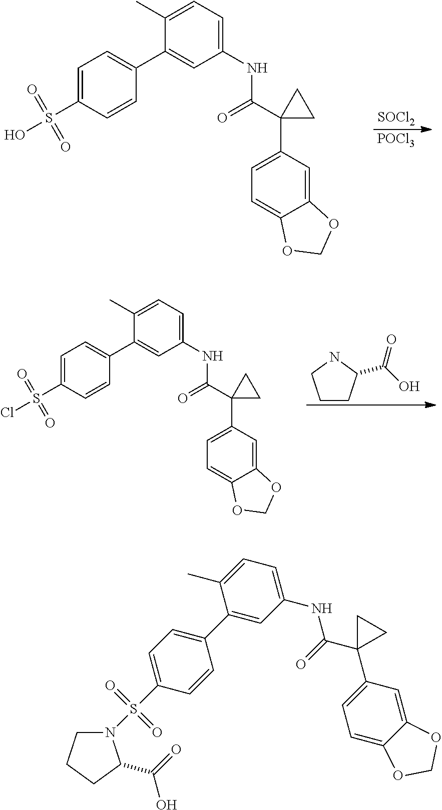

### Step a: 4-(4,4′-Dimethoxybenzhydryl)-thiophenyl boronic acid

4,4′-Dimethoxybenzhydrol (2.7 g, 11 mmol) and 4-mercaptophenylboronic acid (1.54 g, 10 mmol) were dissolved in AcOH (20 mL) and heated at 60° C. for 1 h. Solvent was evaporated and the residue was dried under high vacuum. This material was used without further purification.

### Step b: 4′-[Bis-(4-methoxyphenyl)-methylsulfanyl]-6-methylbiphenyl-3-ylamine

4-(4,4′-Dimethoxybenzhydryl)-thiophenyl boronic acid (10 mmol) and 3-bromo-4-methylaniline (1.86 g, 10 mmol) were dissolved in MeCN (40 mL). Pd (PPh3)4 (˜50 mg) and aqueous solution K2CO3 (1M, 22 mL) were added before the reaction mixture was heated portion-wise in a microwave oven (160° C., 400 sec). Products were distributed between ethyl acetate and water. The organic layer was washed with water, brine and dried over MgSO4. Evaporation yielded an oil that was used without purification in the next step. ESI-MS m/z calc. 441.0. found 442.1 (M+1).

### Step c: 1-Benzo[1,3]dioxol-5-yl-cyclopropanecarboxylic acid 4′-[bis-(4-methoxyphenyl)-methylsulfanyl]-6-methylbiphenyl-3-ylamide

4′-[Bis-(4-methoxyphenyl)-methylsulfanyl]-6-methylbiphenyl-3-ylamine (˜10 mmol) and 1-benzo[1,3]dioxol-5-yl-cyclopropanecarboxylic acid (2.28 g, 11 mmol) were dissolved in chloroform (25 mL) followed by addition of TCPH (4.1 g, 12 mmol) and DIEA (5.0 mL, 30 mmol). The reaction mixture was heated at 65° C. for 48 h. The volatiles were removed under reduced pressure. The residue was distributed between water (200 mL) and ethyl acetate (150 mL). The organic layer was washed with 5% NaHCO3 (2×150 mL), water (1×150 mL), brine (1×150 mL) and dried over MgSO4. Evaporation of the solvent yielded crude 1-benzo[1,3]dioxol-5-yl-cyclopropanecarboxylic acid 4′-[bis-(4-methoxyphenyl)-methylsulfanyl]-6-methylbiphenyl-3-ylamide as a pale oil, which was used without further purification. ESI-MS m/z calc. 629.0. found 630.0 (M+1) (HPLC purity ˜85-90%, UV254 nm).

### Step d: 5′-[(1-Benzo[1,3]dioxol-5-yl-cyclopropanecarbonyl)-amino]-2′-methylbiphenyl-4-sulfonic acid

1-Benzo[1,3]dioxol-5-yl-cyclopropanecarboxylic acid 4′-[bis-(4-methoxyphenyl)-methylsulfanyl]-6-methylbiphenyl-3-ylamide (˜8.5 mmol) was dissolved in acetic acid (75 mL) followed by addition of 30% H2O2 (10 mL). Additional hydrogen peroxide (10 mL) was added 2 h later. The reaction mixture was stirred at 35-45° C. overnight (90% conversion, HPLC). The volume of reaction mixture was reduced to a third by evaporation (bath temperature below 40° C.). The reaction mixture was loaded directly onto a prep RP HPLC column (C-18) and purified. The appropriate fractions with were collected and evaporated to provide 5′-[(1-benzo[1,3]dioxol-5-yl-cyclopropanecarbonyl)-amino]-2′-methylbiphenyl-4-sulfonic acid (2.1 g, 46%, cal. based on 4-mercaptophenylboronic acid). ESI-MS m/z calc. 451.0. found 452.2 (M+1).

### Step e: 5′-[(1-Benzo[1,3]dioxol-5-yl-cyclopropanecarbonyl)-amino]-2′-methylbiphenyl-4-sulfonyl chloride

5′-[(1-Benzo[1,3]dioxol-5-yl-cyclopropanecarbonyl)-amino]-2′-methylbiphenyl-4-sulfonic acid (1.9 g, 4.3 mmol) was dissolved in POCl3 (30 mL) followed by the addition of SOCl2 (3 mL) and DMF (100 μl). The reaction mixture was heated at 70-80° C. for 15 min. The reagents were evaporated and re-evaporated with chloroform-toluene. The residual brown oil was diluted with chloroform (22 mL) and immediately used for sulfonylation. ESI-MS m/z calc. 469.0. found 470.1 (M+1).

### Step f: (S)-1-{5′-[(1-Benzo[1,3]dioxol-5-yl-cyclopropane-carbonyl)-amino]-2′-methyl-biphenyl-4-sulfonyl}-pyrrolidine-2-carboxylic acid

L-Proline (57 mg, 0.50 mmol) was treated with N,O-bis(trimethylsilyl)acetamide (250 μl, 1.0 mmol) in 1 mL dioxane overnight at 50° C. To this mixture was added 5′-(1-(benzo[d][1,3]dioxol-5-yl)cyclopropanecarboxamido)-2′-methylbiphenyl-4-sulfonyl chloride (˜35 μmol, 400 μl solution in chloroform) followed by DIEA (100 μL). The reaction mixture was kept at room temperature for 1 h, evaporated, and diluted with DMSO (404 μl). The resulting solution was subjected to preparative HPLC purification. Fractions containing the desired material were combined and concentrated in vacuum centrifuge at 40° C. to provide the trifluoroacetic salt of (S)-1-{5′-[(1-Benzo[1,3]dioxol-5-yl-cyclopropanecarbonyl)-amino]-2′-methyl-biphenyl-4-sulfonyl}-pyrrolidine-2-carboxylic acid. ESI-MS m/z calc. 548.1. found 549.1 (M+1), retention time 3.40 min; 1H NMR (250 MHz, DMSO-d6) δ 1.04 (m. 2H), δ 1.38 (m, 2H), δ 1.60 (m, 1H), δ 1.80-1.97 (m, 3H) δ 2.16 (s, 3H), δ 3.21 (m, 1H), 3.39 (m, 1H), 4.15 (dd, 1H, J=4.1 Hz, J=7.8 Hz), δ 6.01 (s, 2H), δ 6.89 (s, 2H), δ 6.98 (s, 1H), δ 7.21 (d, 1H, J=8.3 Hz), δ 7.45 (d, 1H, J=2 Hz), δ 7.52 (dd, 1H, J=2 Hz, J=8.3 Hz), δ 7.55 (d, 2H, J=8.3 Hz), δ 7.88 (d, 2H, J=8.3 Hz), δ 8.80 (s, 1H).

The following compounds were prepared using procedure 36 above: 9, 17, 30, 37, 41, 62, 88, 104, 130, 136, 169, 173, 184, 191, 216, 219, 259-60, 265, 275, 278, 281, 302, 306, 342, 350, 366, 371, 380, 387, 396, 404, 412, 430, 438, 449, 460, 478, 486, 496, 499-500, 503, 512, 517, 579, 581-2, 603, 610, 611, 615, 652, 676, 688, 701, 706, 712, 725, 727, 732, 734, 751, 764, 770, 778, 780, 790, 802, 829, 841, 854, 885, 889, 897, 902, 930, 951-2, 970, 986, 992, 994, 997, 1040, 1050-1, 1054, 1056, 1065, 1082, 1090, 1093, 1107, 1114, 1130, 1143, 1147, 1158, 1160, 1164, 1170, 1174-5.

### Preparation 37

5′-(1-(Benzo[d][1,3]dioxol-5-yl)cyclopropanecarboxamido)-2-fluoro-2′-methylbiphenyl-4-carboxamide

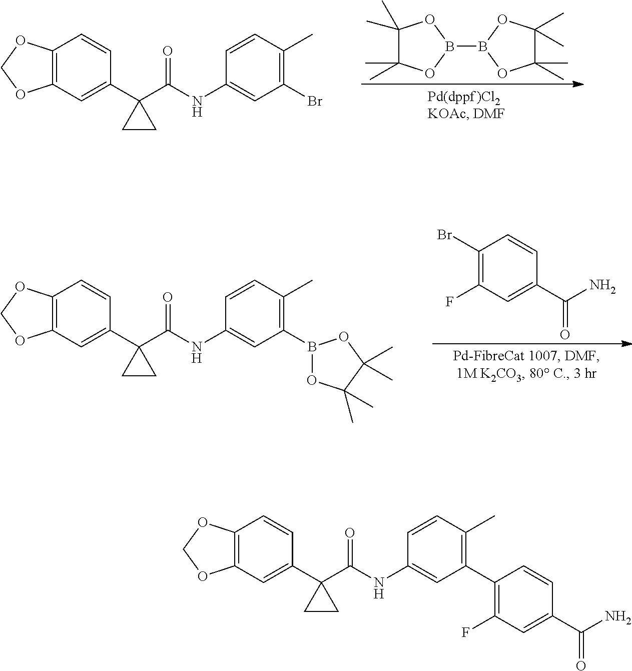

### Step a: 1-(Benzo[d][1,3]dioxol-5-yl)-N-(4-methyl-3-(4,4,5,5-tetramethyl-1,3,2-dioxaborolan-2-yl)phenyl)cyclopropanecarboxamide

1-(Benzo[d][1,3]dioxol-5-yl)-N-(3-bromo-4-methylphenyl)cyclopropanecarboxamide (5.0 g, 13 mmol), 4,4,4′,4′,5,5,5′,5′-octamethyl-2,2′-bi(1,3,2-dioxaborolane) (4.1 g, 16 mmol), Pd(dppf)Cl2 (0.66 g, 0.81 mmol), and DMF (100 mL) were added to a flask containing oven-dried KOAc (3.9 g, 40 mmol). The mixture was heated at 80° C. for 2 h (˜40% conversion). The mixture was cooled to ambient temperature and the volatiles were removed under vacuum. The residue was taken up in CH2Cl2, filtered, and loaded onto a SiO2 column (750 g of SiO2). The product was eluted with EtOAc/Hexanes (0-25%, 70 min, 250 mL/min) to provide 1-(benzo[d][1,3]dioxol-5-yl)-N-(4-methyl-3-(4,4,5,5-tetramethyl-1,3,2-dioxaborolan-2-yl)phenyl)cyclopropanecarboxamide (1.5 g, 27%) and unreacted starting material: 1-(benzo[d][1,3]dioxol-5-yl)-N-(3-bromo-4-methylphenyl)cyclopropanecarboxamide (3.0 g).

### Step b: 5′-(1-(Benzo[d][1,3]dioxol-5-yl)cyclopropanecarboxamido)-2-fluoro-2′-methylbiphenyl-4-carboxamide

1-(Benzo[d][1,3]dioxol-5-yl)-N-(4-methyl-3-(4,4,5,5-tetramethyl-1,3,2-dioxaborolan-2-yl)phenyl)cyclopropanecarboxamide (42 mg, 0.10 mmol), 4-bromo-3-fluorobenzamide (24 mg, 0.11 mmol), Pd-FibreCat 1007 (10 mg), K2CO3 (1M, 240 mL), and DMF (1 mL) were combined in a scintillation vial and heated at 80° C. for 3 hr. The mixture was filtered and purified using reverse-phase preparative HPLC to provide 5′-(1-(benzo[d][1,3]dioxol-5-yl)cyclopropanecarboxamido)-2-fluoro-2′-methylbiphenyl-4-carboxamide (ESI-MS m/z calc. 428.5. found 429.5 (M+1); retention time 3.30 min).

### Preparation 38

### 1-(Benzo[d][1,3]dioxol-5-yl)-N-(6-methyl-3′-(2H-tetrazol-5-yl)biphenyl-3-yl)cyclopropanecarboxamide

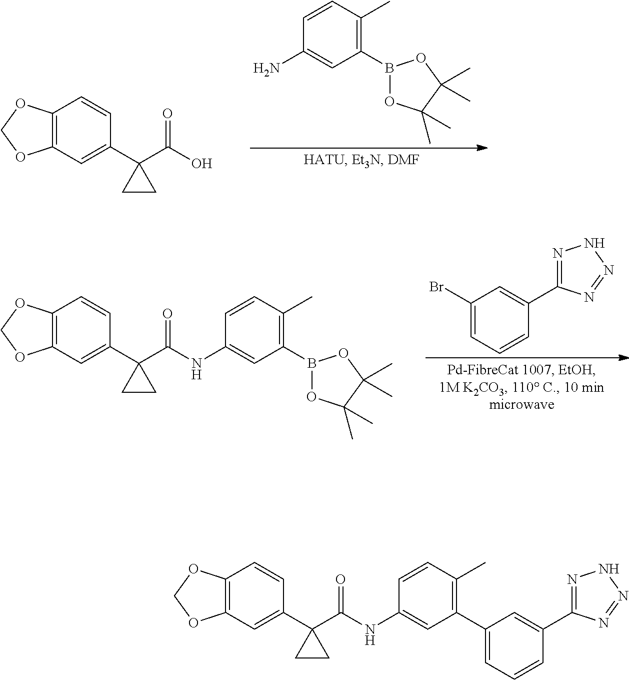 [SMILES: O=C(O)C1(c2ccc3c(c2)OCO3)CC1 ; Cc1ccc(N)cc1B1OC(C)(C)C(C)(C)O1 ; Cc1ccc(NC(=O)C2(c3ccc4c(c3)OCO4)CC2)cc1B1OC(C)(C)C(C)(C)O1 ; Brc1cccc(-c2nn[nH]n2)c1 ; Cc1ccc(NC(=O)C2(c3ccc4c(c3)OCO4)CC2)cc1-c1cccc(-c2nn[nH]n2)c1]

### Step a: 1-(Benzo[d][1,3]dioxol-5-yl)-N-(4-methyl-3-(4,4,5,5-tetramethyl-1,3,2-dioxaborolan-2-yl)phenyl)cyclopropanecarboxamide

To a solution of 1-(benzo[d][1,3]dioxol-5-yl)cyclopropanecarboxylic acid (1.74 g, 8.57 mmol) in DMF (10 mL) was added HATU (3.59 g, 9.45 mmol), Et3N (3.60 mL, 25.8 mmol), then 4-methyl-3-(4,4,5,5-tetramethyl-1,3,2-dioxaborolan-2-yl)aniline (2.19 g, 9.40 mmol) at ambient temperature. The mixture was heated at 70° C. for 18 h. The mixture was cooled, then concentrated under reduced pressure. The residue was taken up in EtOAc before it was washed with H2O, then brine (2×). The organics were dried (Na2SO4) and concentrated under reduced pressure to provide an orange-tan foam/semi-solid. Column chromatography on the residue (5-15% EtOAc/hexanes) provided a white foam. MeOH was added to the material and the slurry was concentrated under reduced pressure to yield 3.10 g of 1-(benzo[d][1,3]dioxol-5-yl)-N-(4-methyl-3-(4,4,5,5-tetramethyl-1,3,2-dioxaborolan-2-yl)phenyl)cyclopropanecarboxamide as a white, granular solid, (85%).

### Step b: 1-(Benzo[d][1,3]dioxol-5-yl)-N-(6-methyl-3′-(2H-tetrazol-5-yl)-biphenyl-3-yl)cyclopropanecarboxamide

1-(Benzo[d][1,3]dioxol-5-yl)-N-(4-methyl-3-(4,4,5,5-tetramethyl-1,3,2-dioxaborolan-2-yl)phenyl)cyclopropanecarboxamide (42.1 mg, 0.100 mmol), 5-(3-bromophenyl)-tetrazole (22.5 mg, 0.100 mmol), a 1 M aqueous solution of potassium carbonate (0.50 mL), Pd-FibreCat 1007 (6 mg), and ethanol (0.50 mL) were combined. The mixture was heated at 110° C. for 5 min (5 min ramp time) in a microwave reactor. After cooling, the mixture was filtered and purified by prep-HPLC to provide 1-(benzo[d][1,3]dioxol-5-yl)-N-(6-methyl-3′-(2H-tetrazol-5-yl)-biphenyl-3-yl)cyclopropanecarboxamide. ESI-MS m/z calc. 439.2. found 440.2 (M+1)+; Retention time 2.59 minutes.

The following compounds were prepared using procedures 13, 24, 32, 34, 37 and 38 above: 1-3, 7-8, 10-13, 15-6, 18-23, 25, 28-9, 31, 33-4, 36, 38, 40, 42-50, 52-54, 56-61, 63-9, 71, 72(1), 73-4, 76-7, 80, 82-3, 85-7, 89, 91-5, 99-100, 102-3, 105-9, 111-113, 115(1), 116-7, 119-21, 123-4, 125(2), 126-9, 131, 133, 135, 137-45, 147-54, 157-8, 160-5, 167-8, 170, 172, 174-5, 176(1), 177-83, 185, 187, 189-90, 193-4, 195(1), 196, 197(1), 198-9, 201-5, 207, 209-10, 212-3, 215, 217, 220-2, 224-9, 231, 232(2), 233-6, 238, 240-4, 246, 249-52, 253(1), 254-7, 262-74, 276-7, 280, 282, 286-8, 290, 293-6, 298-301, 303-5, 307, 310, 312-8, 320-31, 332(2), 333, 335-7, 340, 340, 343-7, 349, 351, 353-4, 357-61, 363-4, 367-70, 372, 374, 375(2), 377(2), 378, 381, 383-6, 388, 390, 394-5, 397-403, 408, 409(2), 413, 414(1), 415-29, 431-2, 434-7, 439-46, 450-1, 454-8, 461, 463-4, 466-8, 469(2), 470, 472-3, 475-6, 479, 480-1, 483-5, 487-93, 497-8, 501-2, 506-7, 509-510, 513, 515-6, 518-21, 523, 525, 527-8, 531-3, 535, 537-8, 539(1), 540-50, 552-3, 555-561, 564-72, 574-6, 578, 583-89, 595-602, 604-5, 606(1), 607-9, 613-4, 616-8, 620, 624-6, 630, 631(1), 632-6, 639-42, 644-7, 649-50, 654-9, 662, 665-7, 670-1, 673-5, 677-80, 683-5, 686(1), 687, 689-91, 693-97, 699-700, 702-3, 705, 708, 711, 713-24, 726, 729(2), 730, 733, 735(1), 738, 741-6, 752-4, 756-63, 765-9, 771-4, 776-7, 779, 781, 784-5, 787-9, 791-6, 798-799, 800(1), 803-5, 807-8, 811, 813, 815-21, 822(1), 823-4, 830-3, 837-40, 847-52, 855-65, 867-70, 872-76, 878-84, 886-8, 890-6, 898-9, 901, 903-4, 908, 910-4, 915(1), 917-25, 927-8, 933-4, 936, 939-40, 942-3, 945-6, 948-9, 953-64, 967-8, 972, 974, 976-7, 980-5, 987-91, 993, 995, 998-1001, 1003, 1005-6, 1008, 1010-11, 1013-32, 1034-6, 1038-9, 1041-5, 1047, 1052-3, 1055, 1057-60, 1062-3, 1067-9, 1071-6, 1078, 1081, 1086-7, 1091-2, 1094-6, 1098-1101, 1103-6, 1108-13, 1115, 1116(2), 1117-26, 1128-9, 1131-40, 1142, 1144-6, 1148-9, 1152-4, 1161, 1165, 1167-9, 1171-3, 1176, 1177(1), 1178-9, 1181-4, 1188-92, 1194, 1197, 1199-1200, 1202-4, 1205(2).

Following the coupling with 2-(3-(4,4,5,5-tetramethyl-1,3,2-dioxaborolan-2-yl)benzyl)isoindoline-1,3-dione and 2-(2-(4,4,5,5-tetramethyl-1,3,2-dioxaborolan-2-yl)benzyl)isoindoline-1,3-dione, examples were obtained after removal of the phthalimide group with hydrazine using known deprotecting procedures.

Following the coupling with 4-((tert-butoxycarbonylamino)methyl)phenylboronic acid, examples were obtained after removal of the Boc-group with TFA using known deprotecting procedures.

### Preparation 39

### 5-(1-(Benzo[d][1,3]dioxol-5-yl)cyclopropanecarboxamido)-N2,N4′,N4′-trimethylbiphenyl-2,4′-dicarboxamide

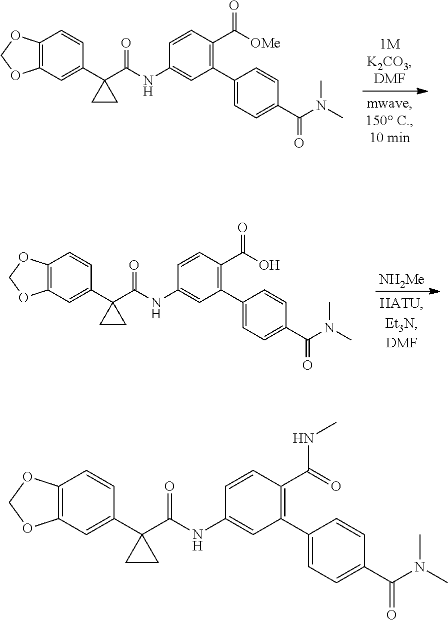 [SMILES: COC(=O)c1ccc(NC(=O)C2(c3ccc4c(c3)OCO4)CC2)cc1-c1ccc(C(=O)N(C)C)cc1 ; CN(C)C(=O)c1ccc(-c2cc(NC(=O)C3(c4ccc5c(c4)OCO5)CC3)ccc2C(=O)O)cc1 ; CNC(=O)c1ccc(NC(=O)C2(c3ccc4c(c3)OCO4)CC2)cc1-c1ccc(C(=O)N(C)C)cc1 ; "NH2Me"]

### Step a: 5-(1-(Benzo[d][1,3]dioxol-5-yl)cyclopropanecarboxamido)-4′-(dimethylcarbamoyl)biphenyl-2-carboxylic acid

Methyl 5-(1-(benzo[d][1,3]dioxol-5-yl)cyclopropanecarboxamido)-4′-(dimethykarbamoyl)biphenyl-2-carboxylate (84 mg, 0.20 mmol) was dissolved in DMF (2.0 mL) with 1M K2CO3 (1.0 mL) and irradiated in the microwave at 150° C. for 10 minutes. Purification by reverse phase HPLC yielded 5-(1-(benzo[d][1,3]dioxol-5-yl)cyclopropanecarboxamido)-4′-(dimethylcarbamoyl)-biphenyl-2-carboxylic acid (7.3 mg, 8%). ESI-MS m/z calc. 472.5. found 473.3 (M+1)+; retention time 2.79 minutes.

### Step b: 5-(1-(Benzo[d][1,3]dioxol-5-yl)cyclopropanecarboxamido)-N2,N4′,N4′-trimethylbiphenyl-2,4′-dicarboxamide

5-(1-(Benzo[d][1,3]dioxol-5-yl)cyclopropanecarboxamido)-4′-(dimethylcarbamoyl) biphenyl-2-carboxylic acid (47 mg, 0.10 mmol) and 75 μL of a 2.0 M solution of methylamine in tetrahydrofuran (0.15 mmol) were dissolved in DMF (1.0 mL) containing Et3N (28 μL, 0.20 mmol). O-(7-Azabenzotriazol-1-yl)-N,N,N′,N_-tetramethyluronium hexafluorophosphate (42 mg, 0.11 mmol) was added to the mixture and the resulting solution was allowed to stir for 3 hours. The mixture was filtered and purified by reverse phase HPLC to yield 5-(1-(benzo[d][1,3]dioxol-5-yl)cyclopropane-carboxamido)-N2,N4′,N4′-trimethylbiphenyl-2,4′-dicarboxamide (5.0 mg, 10%). ESI-MS m/z calc. 485.5. found 486.5 (M+1)+; retention time 2.54 minutes.

The following compounds were prepared using procedure 39 above: 311, 495, 755, 812, 1070.

### Preparation 40

### 5′-(1-(Benzo[d][1,3]dioxol-5-yl)cyclopropanecarboxamido)-2′-((2-hydroxyethylamino)methyl)-N,N-dimethylbiphenyl-4-carboxamide

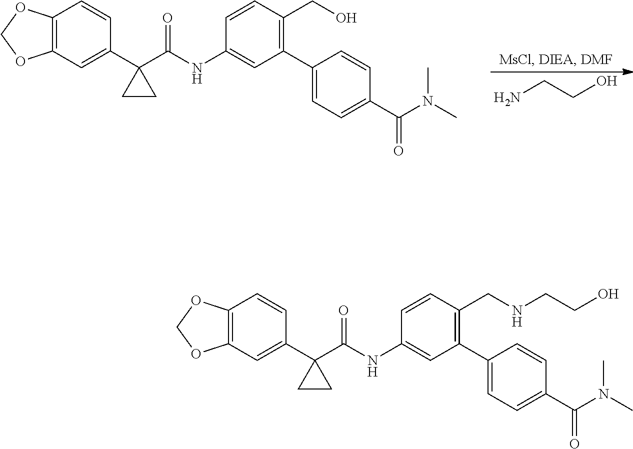

To a solution of 5′-(1-(benzo[d][1,3]dioxol-5-yl)cyclopropanecarboxamido)-2′-(hydroxymethyl)-N,N-dimethylbiphenyl-4-carboxamide (46 mg, 0.10 mmol) and diisopropylethylamine (30 μL, 0.20 mmol) in DMF (1.0 mL) was added methanesulfonyl chloride (8.5 μL, 0.11 mmol). After stirring at 25° C. for 15 minutes, ethanolamine (13 μL, 0.30 mmol) was added and the mixture was stirring for an additional 1 hour. The mixture was filtered and purified by reverse phase HPLC to yield 5′-(1-(benzo[d][1,3]dioxol-5-yl)cyclopropanecarboxamido)-2′-((2-hydroxyethyl-amino)methyl)-N,N-dimethylbiphenyl-4-carboxamide as the trifluoroacetic acid salt (5.0 mg, 8%). ESI-MS m/z calc. 501.2. found 502.5 (M+1)+; retention time 2.28 minutes.

The following compounds were prepared using procedure 40 above: 843, 909, 1080.

### Preparation 41

### 5′-(1-(Benzo[d][1,3]dioxol-5-yl)cyclopropanecarboxamido)-2′-((2-hydroxyethylamino)methyl)-N,N-dimethylbiphenyl-4-carboxamide

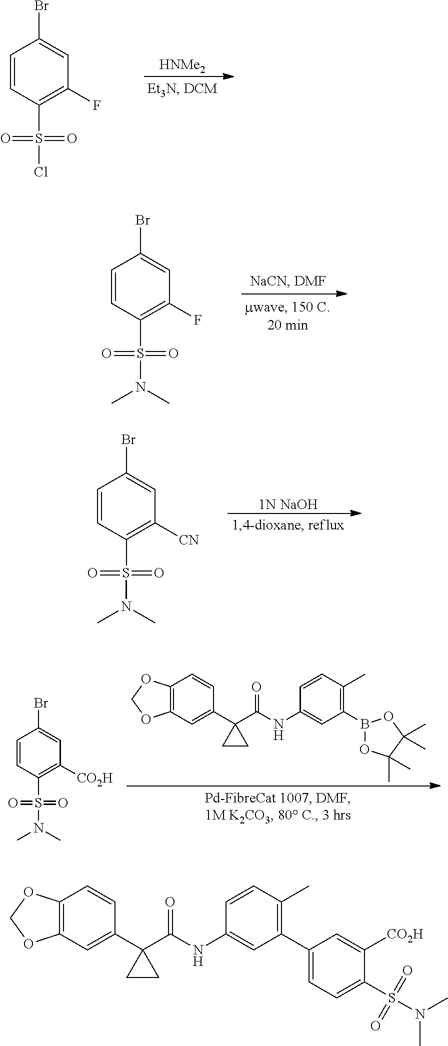

### Step a: 4-Bromo-2-fluoro-N,N-dimethylbenzenesulfonamide

To 4-bromo-2-fluorobenzene-1-sulfonyl chloride (1.0 g, 3.7 mmol) and Et3N (1.5 mL, 11 mmol) in dichloromethane (10 mL) was added a solution of dimethylamine 2.0 M in THF (2.2 mL, 4.4 mmol). The reaction was stirred at ambient temperature for 30 minutes. The reaction was washed with 10 mL of 1N aqueous HCl and 10 mL of brine. Organics were dried over Na2SO4 and evaporated to dryness. Crude product was purified by chromatography on silica gel (eluting with 0-25% ethyl acetate in hexanes) to afford 4-bromo-2-fluoro-N,N-dimethylbenzenesulfonamide (780 mg, 75%).

### Step b: 4-Bromo-2-cyano-N,N-dimethylbenzenesulfonamide

4-Bromo-2-fluoro-N,N-dimethylbenzenesulfonamide (1.0 g, 3.5 mmol) and sodium cyanide (350 mg, 7.1 mmol) were dissolved in DMF (3 mL) and irradiated in the microwave at 150° C. for 20 minutes. DMF was removed in vacuo and the residue was redissolved in dichloromethane (5 mL). The organics were washed with 5 mL of each 1N aqueous HCl, saturated aqueous NaHCO3, and brine. Organics were dried over Na2SO4 and evaporated to dryness. Crude product was purified by chromatography on silica gel (eluting with 0-50% ethyl acetate in hexanes) to afford 4-bromo-2-cyano-N,N-dimethylbenzenesulfonamide (72 mg, 7%). ESI-MS m/z calc. 288.0. found 288.9 (M+1)+; retention time 1.44 minutes.

### Step c: 5-Bromo-2-(N,N-dimethylsulfamoyl)benzoic acid

A mixture of 4-bromo-2-cyano-N,N-dimethylbenzenesulfonamide (110 mg, 0.38 mmol) and 1N aqueous NaOH (2.0 mL, 2.0 mmol) in 1,4-dioxane (2 mL) was heated at reflux. The cooled reaction mixture was washed with dichloromethane (5 mL). The aqueous layer was acidified by the addition of 1N aqueous HCl. The acidified aqueous layer was extracted with dichloromethane (2×5 mL). The combined organics were dried over Na2SO4 and evaporated to dryness to yield 5-bromo-2-(N,N-dimethylsulfamoyl)benzoic acid in 34% yield (40 mg, 0.13 mmol). ESI-MS m/z calc. 307.0. found 308.1 (M+1)+; retention time 1.13 minutes.

### Step d: 5′-(1-(Benzo[d][1,3]dioxol-5-yl)cyclopropanecarboxamido)-4-(N,N-dimethylsulfamoyl)-2′-methylbiphenyl-3-carboxylic acid

1-(Benzo[d][1,3]dioxol-5-yl)-N-(4-methyl-3-(4,4,5,5-tetramethyl-1,3,2-dioxaborolan-2-yl)phenyl)cyclopropanecarboxamide (42 mg, 0.10 mmol), 5-bromo-2-(N,N-dimethylsulfamoyl)benzoic acid (31 mg, 0.10 mmol), 1 M K2CO3 (0.30 mL, 0.30 mmol), and Pd-FibreCat 1007 (8 mg, 0.004 mmol) were dissolved in DMF (1 mL) and heated at 80° C. for 3 hr in an oil bath. The mixture was filtered and purified by reverse phase HPLC to yield 5′-(1-(benzo[d][1,3]dioxol-5-yl)cyclopropanecarboxamido)-4-(N,N-dimethylsulfamoyl)-2′-methylbiphenyl-3-carboxylic acid. ESI-MS m/z calc. 522.6. found 523.5 (M+1)+; retention time 1.79 minutes.

### Preparation 42

### 3-Bromo-4-(3-methyloxetan-3-yl)aniline

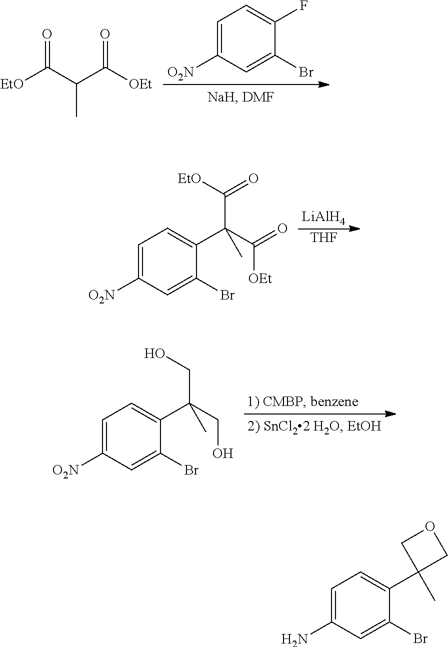

### Step a: Diethyl 2-(2-bromo-4-nitrophenyl)-2-methylmalonate

Diethyl 2-methylmalonate (4.31 mL, 25.0 mmol) was dissolved in 25 mL of anhydrous DMF. This solution was cooled to 0° C. under an atmosphere of nitrogen. Sodium hydride (1.04 g, 26 mmol, 60% by weight in mineral oil) was slowly added to the solution. The resulting mixture was allowed to stir for 3 minutes at 0° C., and then at room temperature for 10 minutes. 2-Bromo-1-fluoro-4-nitrobenzene (5.00 g, 22.7 mmol) was quickly added and the mixture turned bright red. After stirring for 10 minutes at room temperature, the crude mixture was evaporated to dryness and then partitioned between dichloromethane and a saturated aqueous solution of sodium chloride. The layers were separated and the organic phase was washed twice with a saturated aqueous solution of sodium chloride. The organics were concentrated to yield diethyl 2-(2-bromo-4-nitrophenyl)-2-methylmalonate (8.4 g, 99%) as a pale yellow oil which was used without further purification. Retention time 1.86 min.

### Step b: 2-(2-Bromo-4-nitrophenyl)-2-methylpropane-1,3-diol

Diethyl 2-(2-bromo-4-nitrophenyl)-2-methylmalonate (8.12 g, 21.7 mmol) was dissolved in 80 mL of anhydrous tetrahydrofuran (THF) under an atmosphere of nitrogen. The solution was then cooled to 0° C. before a solution of lithium aluminum hydride (23 mL, 23 mmol, 1.0 M in THF) was added slowly. The pale yellow solution immediately turned bright red upon the addition of the lithium aluminum hydride. After 5 min, the mixture was quenched by the slow addition of methanol while maintaining the temperature at 0° C. The reaction mixture was then partitioned between dichloromethane and 1N hydrochloric acid. The layers were separated and the aqueous layer was extracted three times with dichloromethane. The combined organics were evaporated to dryness and then purified by column chromatography (SiO₂, 120 g) utilizing a gradient of 0-100% ethyl acetate in hexanes over 45 minutes. 2-(2-Bromo-4-nitrophenyl)-2-methylpropane-1,3-diol was isolated as a red solid (2.0 g, 31%). 1H NMR (400 MHz, d6-DMSO) [symbol] 8.34 (d, J=2.6 Hz, 1H), 8.16 (dd, J=2.6, 8.9 Hz, 1H), 7.77 (d, J=8.9 Hz, 1H), 4.78 (t, J=5.2 Hz, 2H), 3.98-3.93 (m, 2H), 3.84-3.79 (m, 2H), 1.42 (s, 3H). Retention time 0.89 min.

### Step c: 3-Bromo-4-(3-methyloxetan-3-yl)aniline

2-(2-Bromo-4-nitrophenyl)-2-methylpropane-1,3-diol (0.145 g, 0.500 mmol) was dissolved in 2.5 mL of anhydrous benzene. Cyanomethylenetributylphosphorane (CMBP) (0.181 g, 0.750 mmol) was then added and the solution was allowed to stir at room temperature for 72 hours. The mixture was evaporated to dryness and then re-dissolved in 4 mL of EtOH. Tin(II) chloride dihydrate (0.564 g, 2.50 mmol) was then added and the resulting solution was heated at 70° C. for 1 hour. The mixture was cooled to room temperature and then quenched with a saturated aqueous solution of sodium bicarbonate. The mixture was then extracted three times with ethyl acetate. The combined ethyl acetate extracts were evaporated to dryness and purified by preparative LC/MS to yield 3-bromo-4-(3-methyloxetan-3-yl)aniline as a pale yellow oil (0.032 g, 32%) 1H NMR (400 MHz, CD3CN) [symbol] 7.13 (dd, J=0.7, 1.8 Hz, 1H), 6.94-6.88 (m, 2H), 6.75 (br s, 2H), 4.98 (d, J=5.6 Hz, 2H), 4.51 (d, J=6.1 Hz, 2H), 1.74 (s, 3H). ESI-MS m/z calc. 241.0. found; 242.1 (M+1)+ Retention time 0.53 minutes.

### Preparation 43

3-Bromo-4-ethylaniline

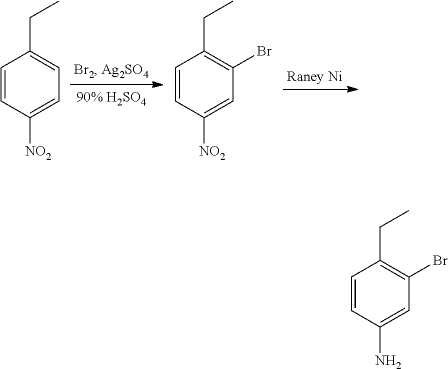

### Step a: 2-Bromo-1-ethyl-4-nitrobenzene

To a mixture of 1-ethyl-4-nitro-benzene (30 g, 0.20 mol), silver sulfate (62 g, 0.20 mol), concentrated sulfuric acid (180 mL) and water (20 g) was added bromine (20 mL, 0.40 mol) dropwise at ambient temperature. After addition, the mixture was stirred for 2 hours at ambient temperature, and then was poured into dilute sodium hydrogen sulfite solution (1 L, 10%). The mixture was extracted with diethylether. The combined organics were dried over Na2SO4 and then concentrated under vacuum to provide a mixture of 2-bromo-1-ethyl-4-nitrobenzene and 1,3-dibromo-2-ethyl-5-nitro-benzene. The mixture was purified by column chromatography (petroleum ether/EtOAc 100:1) to yield 2-bromo-1-ethyl-4-nitrobenzene (25 g) as a yellow oil with a purity of 87%. 1H NMR (300 MHz, CDCl3) δ 8.39 (d, J=2.4 Hz, 1H), 8.09 (dd, J=2.4, 8.4 Hz, 1H), 7.39 (d, J=8.4 Hz, 1H), 2.83 (q, J=7.5 Hz, 2H), 1.26 (t, J=7.5 Hz, 3H).

### Step b: 3-Bromo-4-ethylaniline

To a solution of 2-bromo-1-ethyl-4-nitro-benzene (25 g, 0.019 mol) in MeOH (100 mL) was added Raney-Ni (2.5 g). The reaction mixture was hydrogenated under hydrogen (1 atm) at room temperature. After stirring for 3 hours, the mixture was filtered and concentrated under reduced pressure. The crude material was purified by preparative HPLC to give 3-bromo-4-ethylaniline (8.0 g, 48%). 1H NMR (400 MHz, CDCl3) δ 6.92 (d, J=8.4 Hz, 1H), 6.83 (d, J=2.4 Hz, 1H), 6.52 (dd, J=2.4, 8.4 Hz, 1H), 2.57 (q, J=7.6 Hz, 2H), 1.10 (t, J=7.6 Hz, 3H). MS (ESI) m/e (M+H+) 200.

3-Bromo-4-iso-propylaniline and 3-bromo-4-tert-butylaniline were synthesized following preparation 43 above.

### Preparation 44

### 5-Bromo-2-fluoro-4-methylaniline

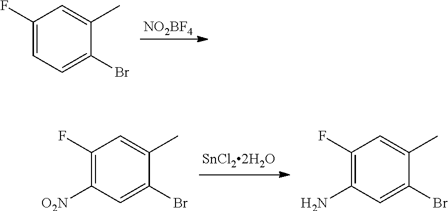

### Step a: 1-Bromo-4-fluoro-2-methyl-5-nitrobenzene

To a stirred solution of 1-bromo-4-fluoro-2-methyl-benzene (15.0 g, 79.8 mmol) in dichloromethane (300 mL) was added nitronium tetrafluoroborate (11.7 g, 87.8 mmol) in portions at 0° C. The mixture was heated at reflux for 5 h and was then poured into ice water. The organic layer was separated and the aqueous phase was extracted with dichloromethane (100 mL×3). The combined organic layers were dried over anhydrous Na2SO4 and evaporated under reduced pressure to give crude 1-bromo-4-fluoro-2-methyl-5-nitrobenzene (18.0 g), which was used directly in the next step.

### Step b: 5-Bromo-2-fluoro-4-methylaniline

To a stirred solution of 1-bromo-4-fluoro-2-methyl-5-nitrobenzene (18.0 g) in ethanol (300 mL) was added SnCl2.2H2O (51.8 g, 0.230 mol) at room temperature. The mixture was heated at reflux for 3 h. The solvent was evaporated under reduced pressure to give a residue, which was poured into ice water. The aqueous phase was basified with sat. NaHCO3 to pH 7. The solid was filtered off and the filtrate was extracted with dichloromethane (200 mL×3). The combined organics were dried over anhydrous Na2SO4 and evaporated under reduced pressure. The residue was purified by column chromatography (petroleum ether/EtOAc=10/1) to afford 5-bromo-2-fluoro-4-methylaniline (5.0 g, 30% yield for two steps). 1H NMR (400 MHz, CDCl3) δ 6.96 (d, J=8.8 Hz, 1H), 6.86 (d, J=11.6 Hz, 1H), 3.64 (br, 2H), 2.26 (s, 3H). MS (ESI) m/z (M+H+) 204.0.

### Preparation 45

### 1-(Benzo[d][1,3]dioxol-5-yl)-N-(3′-chloro-6-methyl-4′-(2H-tetrazol-5-yl)biphenyl-3-yl)cyclopropanecarboxamide

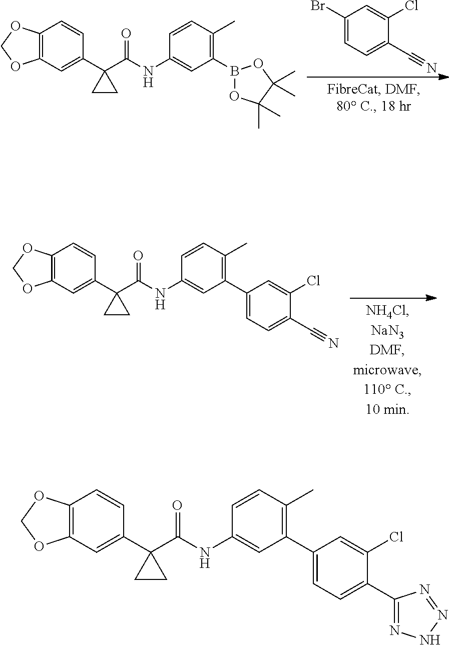 [SMILES: Cc1ccc(NC(=O)C2(c3ccc4c(c3)OCO4)CC2)cc1B1OC(C)(C)C(C)(C)O1 ; N#Cc1ccc(Br)cc1Cl ; Cc1ccc(NC(=O)C2(c3ccc4c(c3)OCO4)CC2)cc1-c1ccc(C#N)c(Cl)c1 ; Cc1ccc(NC(=O)C2(c3ccc4c(c3)OCO4)CC2)cc1-c1ccc(-c2nn[nH]n2)c(Cl)c1]

### Step a: 1-(Benzo[d][1,3]dioxol-5-yl)-N-(3′-chloro-6-methyl-4′-(2H-tetrazol-5-yl)biphenyl-3-yl)cyclopropanecarboxamide

1-(Benzo[d][1,3]dioxol-5-yl)-N-(4-methyl-3-(4,4,5,5-tetramethyl-1,3,2-dioxaborolan-2-yl)phenyl)cyclopropanecarboxamide (0.084 g, 0.20 mmol), 4-bromo-2-chlorobenzonitrile (0.043 g, 0.20 mmol), aqueous potassium carbonate (520 μL, 1M), FibreCat 1007 (7 mg), and DMF (1 mL) were combined. The mixture was heated at 80° C. for 18 hours. After cooling, the mixture was filtered and purified by preparative HPLC to provide 1-(benzo[d][1,3]dioxol-5-yl)-N-(3′-chloro-4′-cyano-6-methylbiphenyl-3-yl)cyclopropanecarboxamide.

### Step b: 1-(Benzo[d][1,3]dioxol-5-yl)-N-(3′-chloro-6-methyl-4′-(2H-tetrazol-5-yl)biphenyl-3-yl)cyclopropanecarboxamide

To 1-(benzo[d][1,3]dioxol-5-yl)-N-(3′-chloro-4′-cyano-6-methylbiphenyl-3-yl)-cyclopropanecarboxamide was added ammonium chloride (0.13 g, 2.4 mmol), sodium azide (0.156 g, 2.40 mmol) and 1 mL of DMF. The mixture was heated at 110° C. in a microwave reactor for 10 minutes. After cooling, the mixture was filtered and purified by preparative HPLC to provide 1-(benzo[d][1,3]dioxol-5-yl)-N-(3′-chloro-6-methyl-4′-(2H-tetrazol-5-yl)biphenyl-3-yl)cyclopropanecarboxamide (8.6 mg, 9%). ESI-MS m/z calc. 473.1. found 474.3 (M+1)+; retention time 1.86 minutes.

### Preparation 46

### 3-Bromo-4-(3-methyloxetan-3-yl)aniline

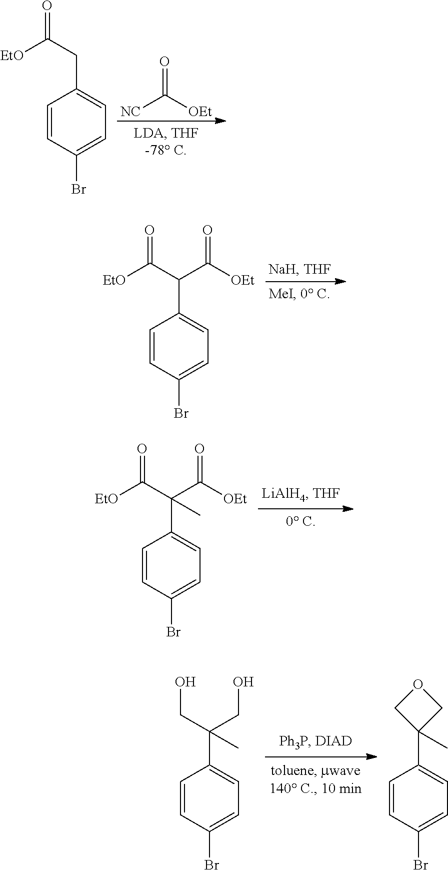

### Step a: Diethyl 2-(4-bromophenyl)malonate

To a solution of ethyl 2-(4-bromophenyl)acetate (5.0 g, 21 mmol) in dry THF (40 mL) at −78° C. was added a 2.0M solution of lithium diisopropylamide in THF (11 mL, 22 mmol). After stirring for 30 minutes at −78° C., ethyl cyanoformate (2.0 mL, 21 mmol) was added and the mixture was allowed to warm to room temperature. After stirring for 48 h at room temperature, the mixture was quenched with water (10 mL). The reaction was partitioned between 1N HCl (50 mL) and dichloromethane (50 mL), and the organic layer was separated. The organic layer was washed with 1N HCl (50 mL), dried over Na2SO4 and evaporated. The crude material was purified by silica gel chromatography, eluting with 0-20% ethyl acetate in hexanes to give diethyl 2-(4-bromophenyl)malonate (2.6 g, 41%) 1H NMR (400 MHz, DMSO-d6) δ 7.60-7.58 (m, 2H), 7.36-7.34 (m, 2H), 5.03 (s, 1H), 4.21-4.09 (m, 4H), 1.20-1.16 (m, 6H).

### Step b: Diethyl 2-(4-bromophenyl)-2-methylmalonate

To a solution of diethyl 2-(4-bromophenyl)malonate (1.5 g, 4.8 mmol) in dry THF (5 mL) at 0° C. was added sodium hydride (380 mg, 9.5 mmol). After stirring for 30 minutes at 0° C., iodomethane (600 μL, 9.5 mmol) was added and the reaction was allowed to warm to room temperature. After stirring for 12 h at room temperature, the reaction was quenched with water (3 mL). The mixture was partitioned between 1N HCl (10 mL) and dichloromethane (10 mL), and the organic layer was separated. The organic layer was washed with 1N HCl (10 mL), dried over Na2SO4 and evaporated. The crude material was purified by silica gel chromatography, eluting with 0-20% ethyl acetate in hexanes, to give diethyl 2-(4-bromophenyl)-2-methylmalonate (850 mg, 55%) 1H NMR (400 MHz, DMSO-d6) δ 7.59-7.55 (m, 2H), 7.31-7.27 (m, 2H), 4.21-4.14 (m, 4H), 1.75 (s, 3H), 1.19-1.16 (m, 6H).

### Step c: 2-(4-Bromophenyl)-2-methylpropane-1,3-diol

To a solution of diethyl 2-(4-bromophenyl)-2-methylmalonate (850 mg, 2.6 mmol) in dry THF (5 mL) at 0° C. was added a 1.0M solution of lithium aluminum hydride in THF (2.6 mL, 2.6 mmol). After stirring for 2 h at 0° C., the mixture was quenched by slow addition of water (5 mL). The mixture was made acidic by addition of 1N HCl and was then extracted with dichloromethane (2×20 mL). The organics were combined, dried over Na2SO4 and evaporated to give 2-(4-bromophenyl)-2-methylpropane-1,3-diol (500 mg, 79%) 1H NMR (400 MHz, DMSO-d6) δ 7.47-7.43 (m, 2H), 7.35-7.32 (m, 2H), 4.59-4.55 (m, 2H), 3.56-3.51 (m, 4H), 1.17 (s, 3H).

### Step d: 3-(4-Bromophenyl)-3-methyloxetane

2-(4-Bromophenyl)-2-methylpropane-1,3-diol (100 mg, 0.41 mmol), triphenyl phosphine (210 mg, 0.82 mmol), and diisopropyl azodicarboxylate (160 μL, 0.82 mmol) were combined in toluene (2 mL) and irradiated in the microwave at 140° C. for 10 minutes. The mixture was directly purified by silica gel chromatography eluting with 0-20% ethyl acetate in hexanes to give 3-(4-bromophenyl)-3-methyloxetane (39 mg, 42%) 1H NMR (400 MHz, DMSO-d6) δ 7.38-7.34 (m, 2H), 7.26-7.22 (m, 2H), 4.82-4.80 (m, 2H), 4.55-4.54 (m, 2H), 1.62 (s, 3H).

### Preparation 47

### N-(4-bromophenylsulfonyl)acetamide

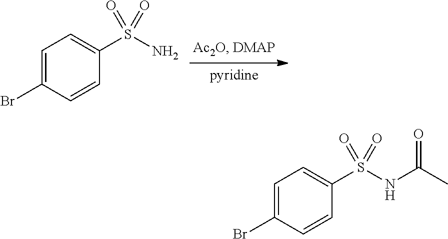

3-Bromobenzenesulfonamide (470 mg, 2.0 mmol) was dissolved in pyridine (1 mL). To this solution was added DMAP (7.3 mg, 0.060 mmol) and then acetic anhydride (570 μL, 6.0 mmol). The reaction was stirred for 3 h at room temperature during which time the reaction changed from a yellow solution to a clear solution. The solution was diluted with ethyl acetate, and then washed with aqueous NH4Cl solution (×3) and water. The organic layer was dried over MgSO4 and concentrated. The resulting oil was triturated with hexanes and the precipitate was collected by filtration to obtain N-(3-bromophenylsulfonyl)-acetamide as a shiny white solid (280 mg, 51%). 1H NMR (400 MHz, DMSO-d6) δ 12.43 (s, 1H), 8.01 (t, J=1.8 Hz, 1H), 7.96-7.90 (m, 2H), 7.61 (t, J=8.0 Hz, 1H), 1.95 (s, 3H); HPLC ret. time 1.06 min; ESI-MS 278.1 m/z (MH+).

### Preparation 48

### 6-Bromoisobenzofuran-1(3H)-one

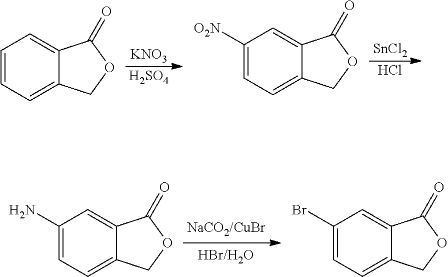

### Step a: 6-Nitroisobenzofuran-1(3H)-one

To a stirred solution of 3H-isobenzofuran-1-one (30.0 g, 0.220 mol) in H2SO4 (38 mL) was added KNO3 (28.0 g, 0.290 mol) in H2SO4 (60 mL) at 0° C. The mixture was stirred at 20° C. for 1 h. The reaction mixture was poured into ice and the resulting precipitate was filtered off. The solid was recrystallized from ethanol to give 6-nitroisobenzofuran-1(3H)-one (32.0 g, 80%). 1H NMR (300 MHz, CDCl3) δ 8.76 (d, J=2.1, 1H), 8.57 (dd, J=8.4, 2.1, 1H), 7.72 (d, J=8.4, 1H), 5.45 (s, 2H).

### Step b: 6-Aminoisobenzofuran-1(3H)-one

To a solution of 6-nitroisobenzofuran-1(3H)-one (15 g, 0.080 mol) in HCl/H2O (375 mL/125 mL) was added SnCl2.2H2O (75 g, 0.33 mol). The reaction mixture was heated at reflux for 4 h before it was quenched with water and extracted with EtOAc (300 mL×3). The organics were dried over Na2SO4 and evaporated in vacuo to give 6-aminoisobenzofuran-1(3H)-one (10 g, 78%). 1H NMR (300 MHz, CDCl3) δ 7.23 (d, J=8.1, 1H), 7.13 (d, J=2.1, 1H), 6.98 (dd, J=8.1, 2.1, 1H), 5.21 (s, 2H), 3.99 (br s, 2H).

### Step c: 6-Bromoisobenzofuran-1(3H)-one

A solution of NaNO2 (2.2 g, 0.040 mol) in H2O (22 mL) was added to a mixture of 6-aminoisobenzofuran-1(3H)-one (5.0 g, 0.030 mol) in HBr (70 mL, 48%) over 5 min at 0° C. The mixture was stirred for 20 minutes before it was pipetted into an ice cold solution of CuBr (22 g, 0.21 mol) in HBr (48%, 23 mL). The resulting dark brown mixture was stirred for 20 min and was then diluted with H₂O (200 mL) to produce an orange precipitate. The precipitate was filtered off, treated with sat. NaHCO₃ solution, and extracted with EtOAc (20 mL×3). The organics were dried over Na2SO4 and evaporated in vacuo to give 6-bromoisobenzofuran-1(3H)-one (5.4 g, 84%). 1H NMR (300 MHz, CDCl3) δ 8.05 (d, J=1.8, 1H), 7.80 (dd, J=8.1, 1.8, 1H), 7.39 (d, J=8.1, 1H), 5.28 (s, 2H).

### Preparation 49

### 6-Bromo-1,1-dioxo-1,2-dihydro-1λ6-benzo[d]isothiazol-3-one

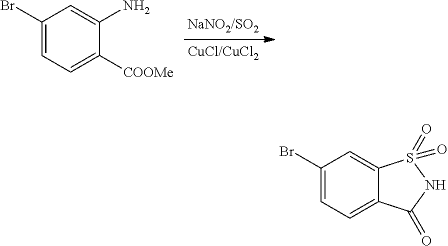 [SMILES: COC(=O)c1ccc(Br)cc1N ; O=C1NS(=O)(=O)c2cc(Br)ccc21]

A solution of methyl 2-amino-4-bromobenzoate (4.5 g, 20 mmol) in 20% hydrochloric acid (30 mL) was stirred until all solids were dissolved. The solution was cooled to 0° C. and a solution of sodium nitrite (1.4 g, 0.020 mol) in water (20 mL) was added dropwise at such a rate that the internal reaction temperature did not exceed 5° C. The mixture was stirred at 0° C. for 45 minutes. Sulfur dioxide was bubbled into a mixture of acetic acid (50 mL) and water (5 mL) at 0° C. until the solution was saturated. Copper (I) chloride (2.0 g, 0.020 mol) was then added to the saturated sulfur dioxide solution. The mixture was cooled to 0° C. To this mixture was added the diazonium salt solution dropwise with vigorous stirring over a period of 30 minutes. The reaction mixture was stirred at 0° C. for 1 hour and then the mixture was allowed to warm to room temperature. The mixture was stirred at room temperature for 2 h before it was poured into ice water (250 mL) and extracted with EtOAc (3×50 mL). The organics were washed with sat. NaHCO₃ solution and dried over anhydrous Na2SO₄. The solvent was removed in vacuo to afford an oily residue which was dissolved in tetrahydrofuran (40 mL) and cooled to 0° C. To this mixture was added a cold (0° C.) solution of ammonium hydroxide (28%, 40 mL) portion-wise at such a rate that the internal reaction temperature was maintained below 10° C. The mixture was allowed to warm to room temperature and was then stirred at room temperature for 1 h. The solvent was removed in vacuo and the residue was dissolved in saturated aqueous sodium bicarbonate (40 mL) and washed with diethyl ether (50 mL). The aqueous layer was acidified with concentrated hydrochloric acid to pH 1. The resulting precipitate was collected by filtration and was dried under vacuum to produce of 6-Bromo-1,1-dioxo-1,2-dihydro-1λ6-benzo[d]isothiazol-3-one (500 mg, 10% yield). 1H NMR (400 MHz, DMSO) δ 8.44 (d, J=1.5, 1H), 8.04 (dd, J=8.1, 1.5, 1H), 7.81 (d, J=8.0, 1H).

### Preparation 50

### 5-Bromo-1,1-dioxo-1,2-dihydro-1λ6-benzo[d]isothiazol-3-one

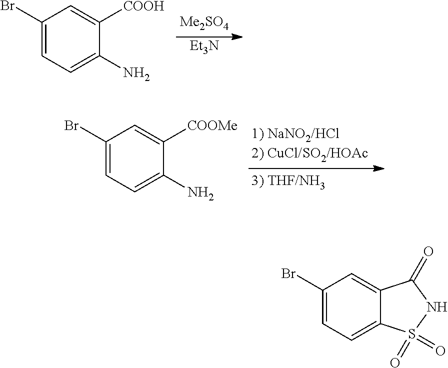 [SMILES: "Et3N" ; Nc1ccc(Br)cc1C(=O)O ; COC(=O)c1cc(Br)ccc1N ; O=C1NS(=O)(=O)c2ccc(Br)cc21]

### Step a: Methyl 2-amino-5-bromobenzoate

MeSO4 (26.3 mL, 0.280 mol) was added to a solution of 2-amino-5-bromobenzoic acid (50.0 g, 0.230 mol) in DMF and Et3N (40 mL, 0.28 mol). The reaction mixture was stirred at rt for 48 h. The mixture was quenched with water, extracted with EtOAc and dried over MgSO4. The solvent was evaporated in vacuo and the residue was purified by chromatography on silica gel (5% EtOAc in petroleum ether) to afford methyl 2-amino-5-bromobenzoate (30 g, 56% yield). 1H NMR (300 MHz, DMSO) δ 7.74 (d, J=2.7, 1H), 7.35 (dd, J=9.0, 2.1, 1H), 6.78-6.73 (m, 3H), 3.77 (s, 3H).

### Step b: 6-Bromo-1,1-dioxo-1,2-dihydro-1λ6-benzo[d]isothiazol-3-one

A solution of the methyl 2-amino-5-bromobenzoate (20.0 g, 86.9 mol) in 20% hydrochloric acid (60 mL) was warmed until all solids were dissolved. The solution was cooled to 0° C. with stirring to precipitate the hydrochloride salt. To this suspension was added a solution of sodium nitrite (6.10 g, 8.84 mol) in water (20 mL) dropwise at such a rate that the internal reaction temperature did not exceed 5° C. The mixture was stirred at 0° C. for 45 minutes to afford a clear solution. Sulfur dioxide was bubbled into a mixture of acetic acid (100 mL) and water (10 mL) at 0° C. Copper (I) chloride (8.6 g, 0.088 mol) was then added to the sulfur dioxide solution. The mixture was then cooled to 0° C. To this mixture was added the diazonium salt solution portion-wise with vigorous stirring over a period of 30 minutes. The reaction mixture was stirred at 0° C. for 1 h and then the mixture was allowed to warm to room temperature. The mixture was stirred at room temperature for 2 h before it was quenched with ice water (500 mL). The mixture was extracted with EtOAc (3×) and the extracts were washed with sat. NaHCO3 and dried over anhydrous Na2SO4. The solvent was removed in vacuo to afford an oily residue. The residue was dissolved in THF (60 mL) and the solution was cooled to 0° C. To this mixture was added a cold (0° C.) solution of sat. NH3 (50 mL) in MeOH portion-wise at such a rate that the internal reaction temperature was maintained below 10° C. After the addition was complete, the mixture was allowed to warm to room temperature and was stirred for 1 h. The solvent was removed in vacuo and the residue was dissolved in saturated aqueous sodium bicarbonate (60 mL) and washed with diethyl ether (80 mL). The aqueous layer was acidified with concentrated HCl to pH to 1. The resulting precipitate was collected by filtration and was dried in vacuo to afford 6-bromo-1,1-dioxo-1,2-dihydro-1λ6-benzo[d]isothiazol-3-one (2.1 g, 9% yield). 1H NMR (300 MHz, CDCl3) δ 8.18 (d, J=1.8, 1H), 8.03 (dd, J=8.1, 1.8, 1H), 7.79 (d, J=8.1, 1H).

### Preparation 51

### 1-(2,2-Difluorobenzo[d][1,3]dioxol-5-yl)-N-(4-methyl-3-(3-oxo-1,3-dihydroisobenzofuran-5-yl)phenyl)cyclopropanecarboxamide and 5′-(1-(2,2-difluorobenzo[d][1,3]dioxol-5-yl)cyclopropanecarboxamido)-4-(hydroxymethyl)-2′-methylbiphenyl-3-carboxylic acid

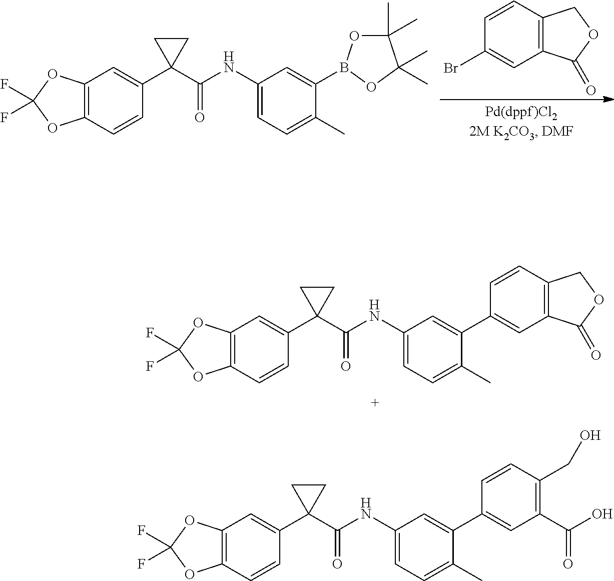

1-(2,2-Difluorobenzo[d][1,3]dioxol-5-yl)-N-(4-methyl-3-(4,4,5,5-tetramethyl-1,3,2-dioxaborolan-2-yl)phenyl)cyclopropanecarboxamide (45 mg, 0.10 mmol), 6-bromoisobenzofuran-1(3H)-one (42 mg, 0.20 mmol), and Pd(dppf)Cl2 (5 mg, 0.006 mmol) were combined in a reaction tube. DMF (1 mL) and 2M K₂CO₃ aqueous solution (250 μL) were added and the mixture was stirred under N2 atmosphere at 80° C. overnight. The mixture was filtered and purified by reverse-phase HPLC (10-99% CH3CN—H2O without TFA modifier) to yield two products: 1-(2,2-difluorobenzo[d][1,3]dioxol-5-yl)-N-(4-methyl-3-(3-oxo-1,3-dihydroisobenzofuran-5-yl)phenyl)cyclopropanecarboxamide: ESI-MS m/z calc. 463.1. found 464.3 (M+1)+. Retention time 2.07 minutes. 1H NMR (400 MHz, DMSO-d6) δ 8.85 (s, 1H), 7.76-7.69 (m, 3H), 7.53-7.48 (m, 2H), 7.42 (d, J=2.2 Hz, 1H), 7.37 (d, J=8.3 Hz, 1H), 7.27 (dd, J=1.7, 8.3 Hz, 1H), 7.22 (d, J=8.4 Hz, 1H), 5.47 (s, 2H), 2.17 (s, 3H), 1.48-1.45 (m, 2H), 1.14-1.11 (m, 2H); and 5′-(1-(2,2-difluorobenzo[d][1,3]dioxol-5-yl)cyclopropanecarboxamido)-4-(hydroxymethyl)-2′-methylbiphenyl-3-carboxylic acid: ESI-MS m/z calc. 481.1. found 482.3 (M+1)+. Retention time 1.84 minutes. 1H NMR (400 MHz, DMSO-d6) δ 8.84 (s, 1H), 8.21 (t, J=6.4 Hz, 1H), 7.67 (d, J=1.9 Hz, 1H), 7.49 (d, J=1.6 Hz, 1H), 7.45 (dd, J=2.2, 8.3 Hz, 1H), 7.37-7.33 (m, 2H), 7.27 (dd, J=1.7, 8.3 Hz, 1H), 7.16-7.10 (m, 3H), 4.44 (d, J=6.2 Hz, 2H), 2.16 (s, 3H), 1.48-1.45 (m, 2H), 1.12-1.09 (m, 2H).

### Preparation 52

### 5′-(1-(2,2-Difluorobenzo[d][1,3]dioxol-5-yl)cyclopropanecarboxamido)-2′-methyl-N-(methylsulfonyl)biphenyl-3-carboxamide

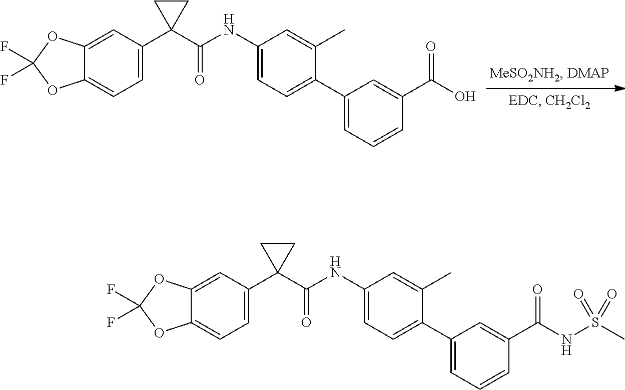 [SMILES: Cc1cc(NC(=O)C2(c3ccc4c(c3)OC(F)(F)O4)CC2)ccc1-c1cccc(C(=O)O)c1 ; Cc1cc(NC(=O)C2(c3ccc4c(c3)OC(F)(F)O4)CC2)ccc1-c1cccc(C(=O)NS(C)(=O)=O)c1]

To a mixture of 5′-(1-(2,2-difluorobenzo[d][1,3]dioxol-5-yl)cyclopropanecarboxamido)-2′-methylbiphenyl-3-carboxylic acid (50 mg, 0.11 mmol), methansulfonamide (7.0 mg, 0.074 mmol), DMAP (13 mg, 0.11 mmol), and CH2Cl2 (1 mL) was added EDC (28 mg, 0.15 mmol) at ambient temperature. The mixture was allowed to stir for 18 h before it was concentrated. The residue was taken up in DMF (1 mL) and was purified by reverse phase preparatory HPLC to provide 5′-(1-(2,2-difluorobenzo[d][1,3]dioxol-5-yl)cyclopropanecarboxamido)-2′-methyl-N-(methylsulfonyl)biphenyl-3-carboxamide as a white solid. ESI-MS m/z calc. 528.5. found 529.2 (M+1)+. Retention time 1.97 minutes.

### Preparation 53

### 1-(2,2-Difluoro-2H-1,3-benzodioxol-5-yl)-N-[4-methyl-3-(1,1,3-trioxo-2,3-dihydro-1λ6,2-benzothiazol-5-yl)phenyl]cyclopropane-1-carboxamide

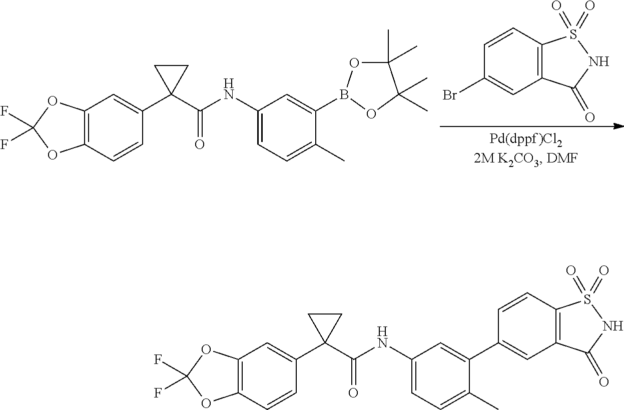

1-(2,2-Difluorobenzo[d][1,3]dioxol-5-yl)-N-(4-methyl-3-(4,4,5,5-tetramethyl-1,3,2-dioxaborolan-2-yl)phenyl)cyclopropanecarboxamide (46 mg, 0.10 mmol), 5-bromo-1,1-dioxo-1,2-dihydro-1λ6-benzo[d]isothiazol-3-one (26 mg, 0.10 mmol), Pd(dppf)Cl₂ (4.0 mg, 0.0050 mmol), 2M Na₂CO₃ (150 μL, 0.30 mmol), and DMF (1 mL) were combined and heated at 120° C. in the microwave for 10 min. The mixture was filtered and purified by reverse phase preparatory HPLC to give 1-(2,2-difluoro-2H-1,3-benzodioxol-5-yl)-N-[4-methyl-3-(1,1,3-trioxo-2,3-dihydro-1λ6,2-benzothiazol-5-yl)phenyl]cyclopropane-1-carboxamide. ESI-MS m/z calc. 512.5. found 513.1 (M+1)+. Retention time 1.94 minutes.

### Preparation 54

### 1-(2,2-Difluoro-2H-1,3-benzodioxol-5-yl)-N-[4-methyl-3-(1,1,3-trioxo-2,3-dihydro-1λ6,2-benzothiazol-6-yl)phenyl]cyclopropane-1-carboxamide

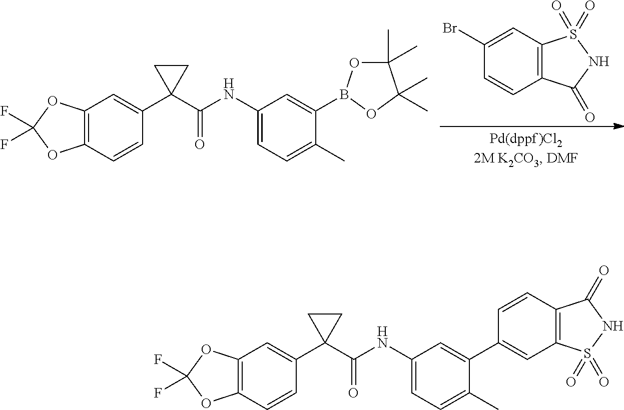

1-(2,2-Difluorobenzo[d][1,3]dioxol-5-yl)-N-(4-methyl-3-(4,4,5,5-tetramethyl-1,3,2-dioxaborolan-2-yl)phenyl)cyclopropanecarboxamide (46 mg, 0.10 mmol), 6-bromo-1,1-dioxo-1,2-dihydro-1λ6-benzo[d]isothiazol-3-one (26 mg, 0.10 mmol), Pd(dppf)Cl₂ (4.0 mg, 0.0050 mmol), 2M Na₂CO₃ (150 μL, 0.30 mmol), and DMF (1 mL) were combined and heated at 120° C. in the microwave for 10 min. The mixture was filtered and purified by reverse phase preparatory HPLC to give 1-(2,2-difluoro-2H-1,3-benzodioxol-5-yl)-N-[4-methyl-3-(1,1,3-trioxo-2,3-dihydro-1λ6,2-benzothiazol-6-yl)phenyl]cyclopropane-1-carboxamide. ESI-MS m/z calc. 512.5. found 513.5 (M+1)+. Retention time 1.93 minutes.

### II.C. Embodiments of Column C Compounds

The modulators of ABC transporter activity in Column C are fully described and exemplified in U.S. Pat. Nos. 7,741,321 and 7,659,268, and also in U.S. patent application Ser. No. 12/114,935, published as US 2008/0306062 A1. All of which are commonly assigned to the Assignee of the present invention. All of the compounds recited in the above publications are useful in the present invention and are hereby incorporated into the present disclosure in their entirety.

### II.C.1 Compounds of Formula C

The present invention includes a compound of Formula C,

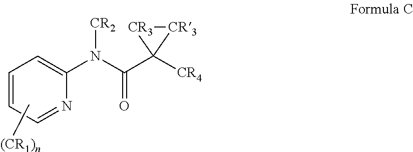

or a pharmaceutically acceptable salt thereof, wherein:

Each CR₁ is a an optionally substituted C₁-C₆ aliphatic, an optionally substituted aryl, an optionally substituted heteroaryl, an optionally substituted 3 to 10 membered cycloaliphatic, an optionally substituted 3 to 10 membered heterocycloaliphatic, carboxy [e.g., hydroxycarbonyl or alkoxycarbonyl], amido [e.g., aminocarbonyl], amino, halo, or hydroxy, provided that at least one R₁ is an optionally substituted aryl or an optionally substituted heteroaryl attached to the 5- or 6-position of the pyridyl ring.

Each CR₂ is hydrogen, an optionally substituted C₁₋₆ aliphatic, an optionally substituted C₃₋₆ cycloaliphatic, an optionally substituted phenyl, or an optionally substituted heteroaryl.

Each CR₃ and CR′₃ together with the carbon atom to which they are attached form an optionally substituted C₃₋₇ cycloaliphatic or an optionally substituted heterocycloaliphatic.

Each CR₄ is an optionally substituted aryl or an optionally substituted heteroaryl.

Each n is 1-4.

### B. Specific Embodiments

### 1. Substituent CR₁

Each CR₁ is an optionally substituted C₁-C₆ aliphatic, an optionally substituted aryl, an optionally substituted heteroaryl, an optionally substituted 3 to 10 membered cycloaliphatic, an optionally substituted 3 to 10 membered heterocycloaliphatic, carboxy [e.g., hydroxycarbonyl or alkoxycarbonyl], amido [e.g., aminocarbonyl], amino, halo, or hydroxy.

In several embodiments, CR₁ is an aryl or heteroaryl with 1-3 substituents. In several examples, R₁ is a monocyclic aryl or heteroaryl.

In several embodiments, at least one CR₁ is an aryl or a heteroaryl and CR₁ is bonded to the core structure at the 6 position on the pyridine ring.

In several embodiments, at least one CR₁ is an aryl or a heteroaryl and CR₁ is bonded to the core structure at the 5 position on the pyridine ring.

In several embodiments, CR₁ is phenyl with up to 3 substituents.

In several embodiments, CR₁ is a heteroaryl ring with up to 3 substituents. In certain embodiments, CR₁ is a monocyclic heteroaryl ring with up to 3 substituents. In other embodiments, CR₁ is a bicyclic heteroaryl ring with up to 3 substituents

In several embodiments, CR₁ is substituted with no more than three substituents selected from halo, oxo, or optionally substituted aliphatic, cycloaliphatic, heterocycloaliphatic, amino [e.g., (aliphatic)amino], amido [e.g., aminocarbonyl, ((aliphatic)amino)carbonyl, and ((aliphatic)₂amino)carbonyl], carboxy [e.g., alkoxycarbonyl and hydroxycarbonyl], sulfamoyl [e.g., aminosulfonyl, ((aliphatic)₂amino)sulfonyl, ((cycloaliphatic)aliphatic)aminosulfonyl, and ((cycloaliphatic)amino)sulfonyl], cyano, alkoxy, aryl, heteroaryl [e.g., monocyclic heteroaryl and bicycloheteroaryl], sulfonyl [e.g., aliphaticsulfonyl or (heterocycloaliphatic)sulfonyl], sulfonyl [e.g., aliphaticsulfinyl], aroyl, heteroaroyl, or heterocycloaliphaticcarbonyl.

In several embodiments, CR₁ is substituted with an optionally substituted aliphatic. Examples of CR₁ substituents include optionally substituted alkoxyaliphatic, heterocycloaliphatic, aminoalkyl, hydroxyalkyl, (heterocycloalkyl)aliphatic, alkylsulfonylaliphatic, alkylsulfonylaminoaliphatic, alkylcarbonylaminoaliphatic, alkylaminoaliphatic, or alkylcarbonylaliphatic.

In several embodiments, CR₁ is substituted with an optionally substituted amino. Examples of CR₁ substituents include aliphaticcarbonylamino, aliphaticamino, arylamino, or aliphaticsulfonylamino.

In several embodiments, CR₁ is substituted with a sulfonyl. Examples of CR₁ substituents include heterocycloaliphaticsulfonyl, aliphatic sulfonyl, aliphaticaminosulfonyl, aminosulfonyl, aliphaticcarbonylaminosulfonyl, alkoxyalkylheterocycloalkylsulfonyl, alkylheterocycloalkylsulfonyl, alkylaminosulfonyl, cycloalkylaminosulfonyl, (heterocycloalkyl)alkylaminosulfonyl, and heterocycloalkylsulfonyl.

In several embodiments, CR₁ is substituted with carboxy. Examples of CR₁ substituents include alkoxycarbonyl and hydroxycarbonyl.

In several embodiments CR₁ is substituted with amido. Examples of CR₁ substituents include alkylaminocarbonyl, aminocarbonyl, ((aliphatic)₂amino)carbonyl, and [((aliphatic)aminoaliphatic)amino]carbonyl.

In several embodiments, CR₁ is substituted with arylcarbonyl, cycloaliphaticcarbonyl, heterocycloaliphaticcarbonyl, or heteroarylcarbonyl.

In some embodiments, CR₁ is hydrogen, or —Z^{A}CR₅, wherein each Z^{A} is independently a bond or an optionally substituted branched or straight C₁₋₆ aliphatic chain wherein up to two carbon units of Z^{A} are optionally and independently replaced by —CO—, —CS—, —CONR^{A}—, —CONR^{A}NR^{A}—, —CO₂—, —OCO—, —NR^{A}CO₂—, —O—, —NR^{A}CONR^{A}—, —OCONR^{A}—, —NR^{A}NR^{A}—, —NR^{A}CO—, —S—, —SO—, —SO₂—, —NR^{A}—, —SO₂NR^{A}—, —NR^{A}SO₂—, or —NR^{A}SO₂NR^{A}—. Each CR₅ is independently R^{A}, halo, —OH, —NH₂, —NO₂, —CN, or —OCF₃. Each R^{A} is independently a C₁₋₈ aliphatic group, a cycloaliphatic, a heterocycloaliphatic, an aryl, or a heteroaryl, each of which is optionally substituted with 1 to 3 of CR^{D}. Each CR^{D} is —Z^{D}CR₉, wherein each Z^{D} is independently a bond or an optionally substituted branched or straight C₁₋₆ aliphatic chain wherein up to two carbon units of Z^{D} are optionally and independently replaced by —CO—, —CS—, —CONR^{E}—, —CONR^{E}NR^{E}—, —CO₂—, —OCO—, —NR^{E}CO₂—, —O—, —NR^{E}CONR^{E}—, —OCONR^{E}—, —NR^{E}NR^{E}—, —NR^{E}CO—, —S—, —SO—, —SO₂—, —SO₂NR^{E}—, —NR^{E}SO₂—, or —NR^{E}SO₂NR^{E}—. Each CR₉ is independently R^{E}, halo, —OH, —NH₂, —NO₂, —CN, or —OCF₃. Each R^{E} is independently hydrogen, an optionally substituted C₁₋₈ aliphatic group, an optionally substituted cycloaliphatic, an optionally substituted heterocycloaliphatic, an optionally substituted aryl, or an optionally substituted heteroaryl.

In some embodiments, one CR₁ is aryl or heteroaryl, each optionally substituted with 1 to 3 of R^{D}, wherein R^{D} is defined above.

In several embodiments, one R₁ is carboxy [e.g., hydroxycarbonyl or alkoxycarbonyl], amido [e.g., aminocarbonyl], amino, halo, cyano, or hydroxyl.

In several embodiments, CR₁ is:

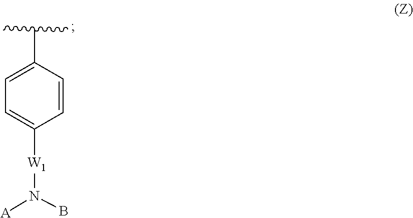

wherein:

W₁ is —C(O)—, —SO₂—, or —CH₂—;

Each of A and B is independently H, an optionally substituted C₁-C₆ aliphatic, an optionally substituted C₃-C₈ cycloaliphatic; or

A and B, taken together, form an optionally substituted 3-7 membered heterocycloaliphatic ring.

In several embodiments, W₁ is —C(O)—. Or, W₁ is —SO₂—. Or, W₁ is —CH₂—.

In several embodiments, A is H and B is an optionally substituted C₁-C₆ aliphatic. Or, both, A and B, are H. Exemplary substituents include oxo, alkyl, hydroxy, hydroxyalkyl, alkoxy, alkoxyalkyl, or an optionally substituted heterocycloaliphatic.

In several embodiments, A and B, taken together, form an optionally substituted 3-7 membered heterocycloaliphatic ring. Exemplary such rings include optionally substituted pyrrolidinyl, piperidinyl, morpholinyl, or piperazinyl. Exemplary substituents on such rings include oxo, alkyl, hydroxy, hydroxyalkyl, alkoxy, alkoxyalkyl, halo, acyl (e.g., alkylcarbonyl), or amido.

In several examples, CR₁ is one selected from:

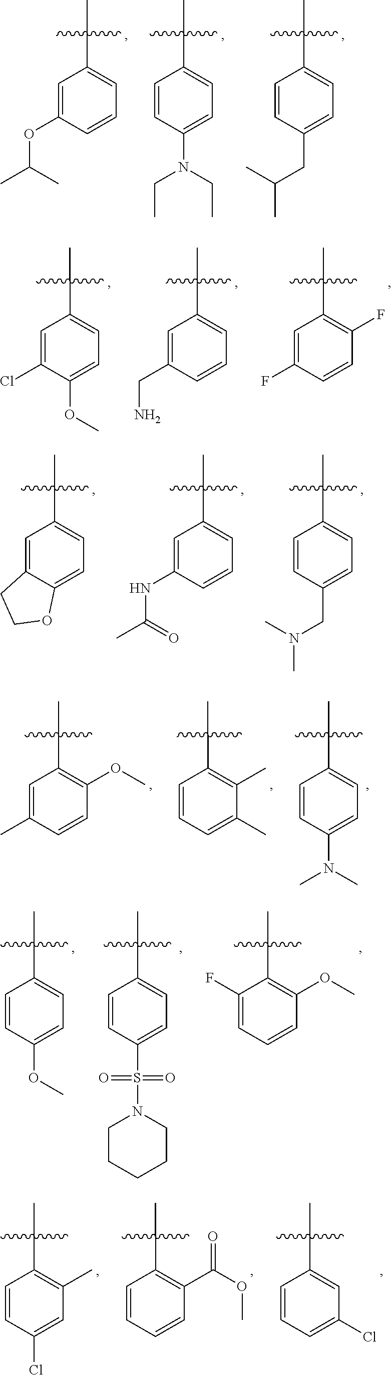

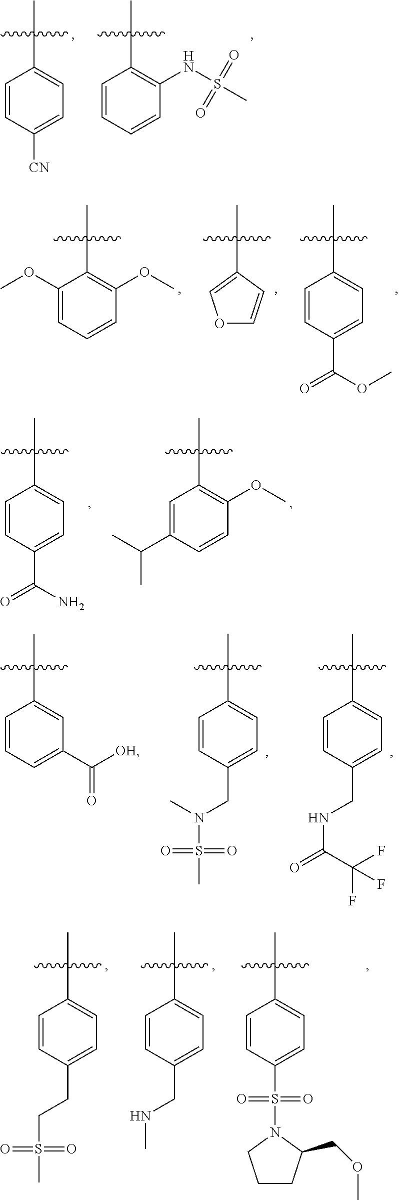

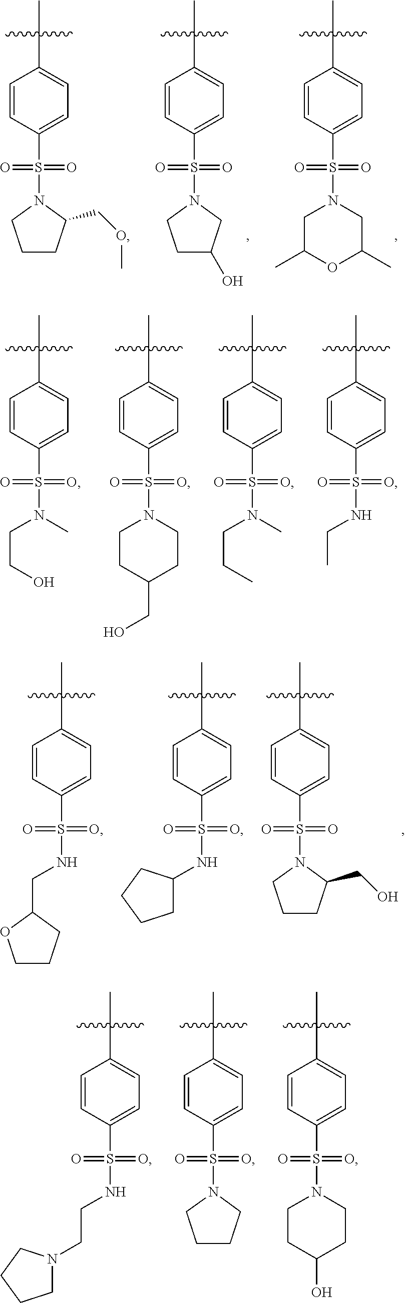

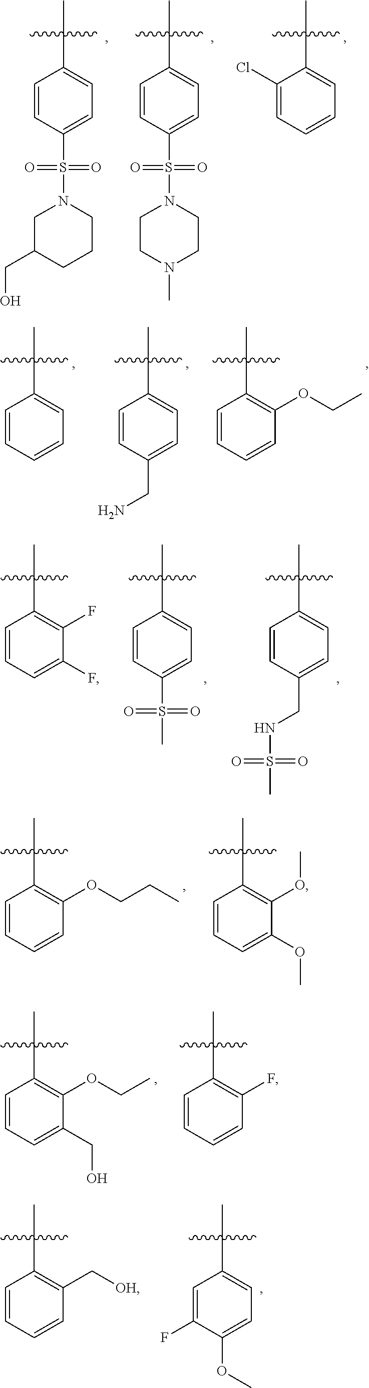

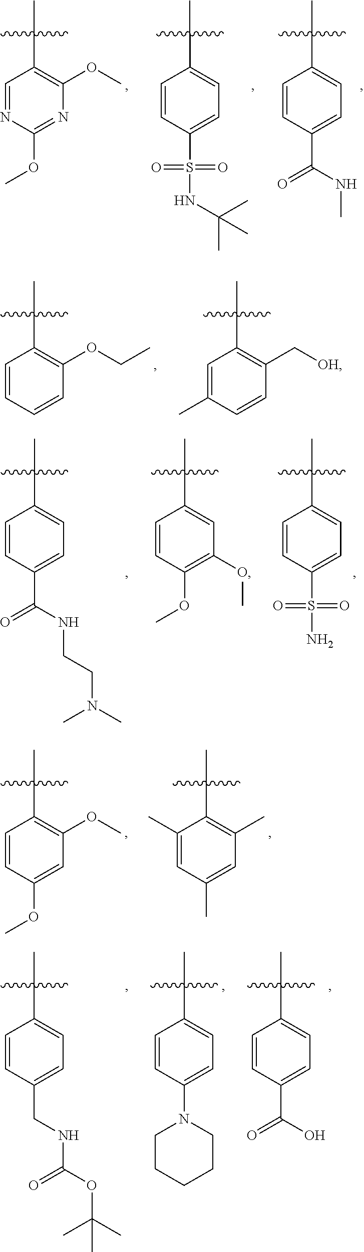

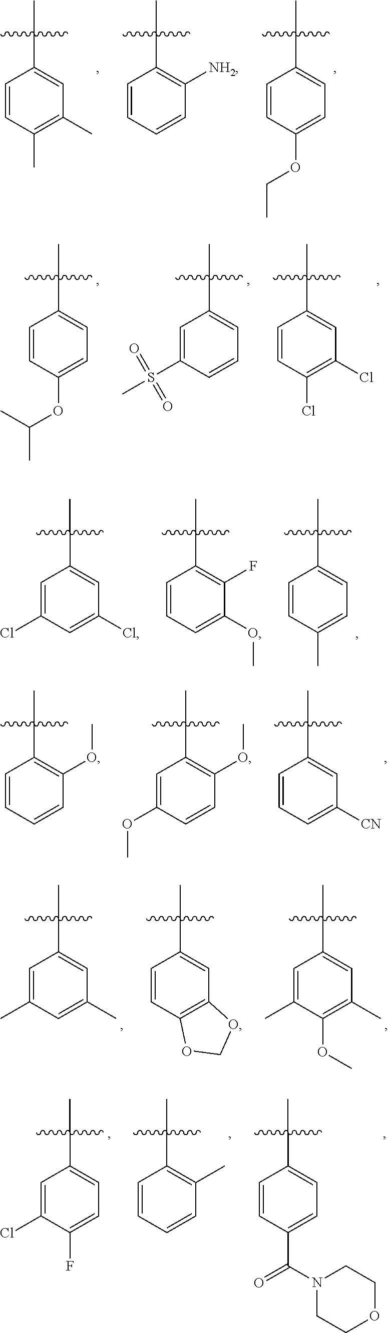

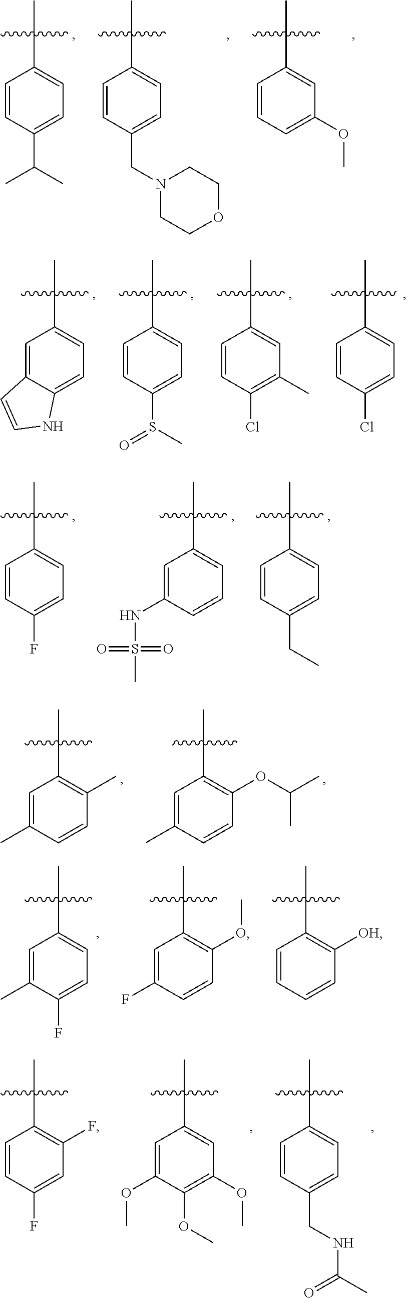

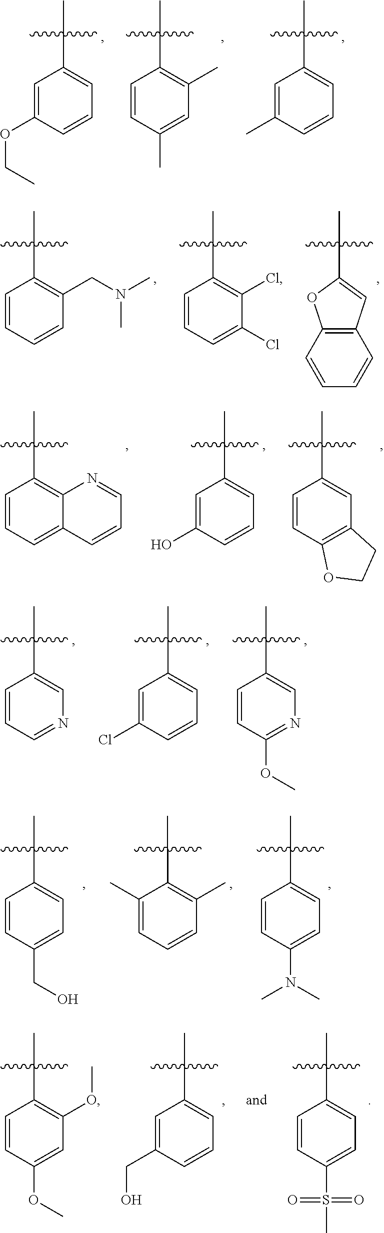

### 2. Substituent CR₂

Each CR₂ is hydrogen, or optionally substituted C₁₋₆ aliphatic, C₃₋₆ cycloaliphatic, phenyl, or heteroaryl.

In several embodiments, CR₂ is a C₁₋₆ aliphatic that is optionally substituted with 1-3 halo, C₁₋₂ aliphatic, or alkoxy. In several examples, R₂ is substituted or unsubstituted methyl, ethyl, propyl, or butyl.

In several embodiments, CR₂ is hydrogen.

### 3. Substituents CR₃ and CR′₃

Each CR₃ and CR′₃ together with the carbon atom to which they are attached form a C₃₋₇ cycloaliphatic or a heterocycloaliphatic, each of which is optionally substituted with 1-3 substituents.

In several embodiments, CR₃ and CR′₃ together with the carbon atom to which they are attached form a C₃₋₇ cycloaliphatic or a C₃₋₇ heterocycloaliphatic, each of which is optionally substituted with 1-3 of —Z^{B}CR₇, wherein each Z^{B} is independently a bond, or an optionally substituted branched or straight C₁₋₄ aliphatic chain wherein up to two carbon units of Z^{B} are optionally and independently replaced by —CO—, —CS—, —CONCR^{B}—, —CONCR^{B}NCR^{B}—, —CO₂—, —OCO—, —NCR^{B}CO₂—, —O—, —NCR^{B}CONCR^{B}—, —OCONCR^{B}—, —NCR^{B}NCR^{B}—, —NCR^{B}CO—, —S—, —SO—, —SO₂—, —NCR^{B}—, —SO₂NCR^{B}—, —NCR^{B}SO₂—, or —NCR^{B}SO₂NCR^{B}—; each CR₇ is independently CR⁸, halo, —OH, —NH₂, —NO₂, —CN, or —OCF₃; and each CR^{B} is independently hydrogen, an optionally substituted C₁₋₈ aliphatic group; an optionally substituted cycloaliphatic, an optionally substituted heterocycloaliphatic, an optionally substituted aryl, or an optionally substituted heteroaryl.

In several embodiments, CR₃ and CR′₃ together with the carbon atom to which they are attached form a 3, 4, 5, or 6 membered cycloaliphatic that is optionally substituted with 1-3 substituents. In several examples, CR₃, CR′₃, and the carbon atom to which they are attached form an optionally substituted cyclopropyl group. In several alternative examples, CR₃, CR′₃, and the carbon atom to which they are attached form an optionally substituted cyclobutyl group. In several other examples, CR₃, CR′₃, and the carbon atom to which they are attached form an optionally substituted cyclopentyl group. In other examples, CR₃, CR′₃, and the carbon atom to which they are attached form an optionally substituted cyclohexyl group. In more examples, CR₃ and CR′₃ together with the carbon atom to which they are attached form an unsubstituted cyclopropyl.

In several embodiments, CR₃ and CR′₃ together with the carbon atom to which they are attached form a 5, 6, or 7 membered optionally substitute heterocycloaliphatic. In other examples, CR₃, CR′₃, and the carbon atom to which they are attached form an optionally substituted tetrahydropyranyl group.

### 4. Substituent CR₄

Each CR₄ is independently an optionally substituted aryl or heteroaryl.

In several embodiments, CR₄ is an aryl including 6 to 10 members (e.g., 7 to 10 members) optionally substituted with 1 to 3 substituents. Examples of CR₄ are optionally substituted benzene, naphthalene, or indene.

In several embodiments, CR₄ is an optionally substituted heteroaryl. Examples of CR₄ include monocyclic and bicyclic heteroaryl, such a benzofused ring system in which the phenyl is fused with one or two C₄₋₈ heterocycloaliphatic groups.

In some embodiments, CR₄ is an aryl or heteroaryl, each optionally substituted with 1-3 of —Z^{C}CR₈. Each Z^{C} is independently a bond or an optionally substituted branched or straight C₁₋₆ aliphatic chain wherein up to two carbon units of Z^{C} are optionally and independently replaced by —CO—, —CS—, —CONCR^{C}—, —CONCR^{C}NCR^{C}—, —CO₂—, —OCO—, —NR^{C}CO₂—, —O—, —NCR^{C}CONCR^{C}—, —OCONCR^{C}—, —NCR^{C}NCR^{C}—, —NCR^{C}CO—, —S—, —SO—, —SO₂—, —NCR^{C}—, —SO₂NCR^{C}—, —NCR^{C}SO₂—, or —NCR^{C}SO₂NCR^{C}—. Each CR₈ is independently CR^{C}, halo, —OH, —NH₂, —NO₂, —CN, or —OCF₃. Each CR^{C} is independently hydrogen, an optionally substituted C₁₋₈ aliphatic group; an optionally substituted cycloaliphatic, an optionally substituted heterocycloaliphatic, an optionally substituted aryl, an optionally substituted heteroaryl.

In several embodiments, CR₄ is one selected from

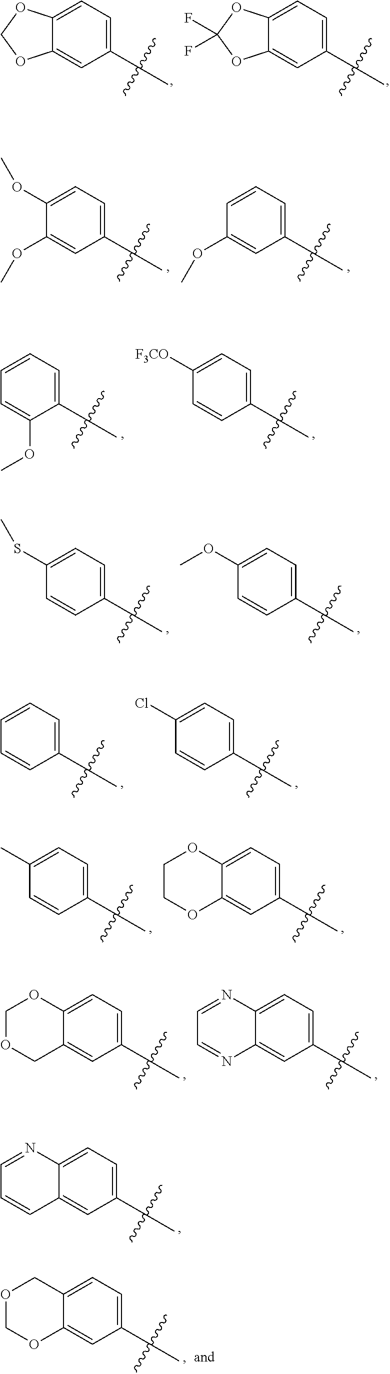

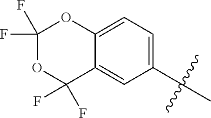 [SMILES: *c1ccc2c(c1)C(F)(F)OC(F)(F)O2]

### 5. Exemplary Compound Families

In several embodiments, CR₁ is an optionally substituted cyclic group that is attached to the core structure at the 5 or 6 position of the pyridine ring.

In several examples, CR₁ is an optionally substituted aryl that is attached to the 5 position of the pyridine ring. In other examples, CR₁ is an optionally substituted aryl that is attached to the 6 position of the pyridine ring.

In more examples, CR₁ is an optionally substituted heteroaryl that is attached to the 5 position of the pyridine ring. In still other examples, CR₁ is an optionally substituted heteroaryl that is attached to the 6 position of the pyridine ring.

In other embodiments, CR₁ is an optionally substituted cycloaliphatic or heterocycloaliphatic that is attached to the pyridine ring at the 5 or 6 position.

Accordingly, another aspect of the present invention provides compounds of Formula (CII):

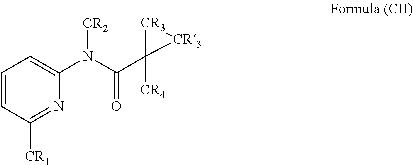

or a pharmaceutically acceptable salt thereof, wherein CR₂, CR₃, CR′₃, and CR₄ are defined in Formula C.

Each CR₁ is aryl or heteroaryl optionally substituted with 1 to 3 of CR^{D}, wherein CR^{D} is —Z^{D}CR₉, wherein each Z^{D} is independently a bond or an optionally substituted branched or straight C₁₋₆ aliphatic chain wherein up to two carbon units of Z^{D} are optionally and independently replaced by —CO—, —CS—, —CONCR^{E}—, —CONCR^{E}NCR^{E}—, —CO₂—, —OCO—, —NCR^{E}CO₂—, —O—, —NCR^{E}CONCR^{E}—, —OCONCR^{E}—, —NCR^{E}NCR^{E}—, —NCR^{E}CO—, —S—, —SO—, —SO₂—, —NCR^{E}—, —SO₂NCR^{E}—, —NCR^{E}SO₂—, or —NCR^{E}SO₂NCR^{E}—; each CR₉ is independently CR^{E}, halo, —OH, —NH₂, —NO₂, —CN, or —OCF₃; each CR^{E} is independently hydrogen, an optionally substituted C₁₋₈ aliphatic group, an optionally substituted cycloaliphatic, an optionally substituted heterocycloaliphatic, an optionally substituted aryl, or an optionally substituted heteroaryl.

Another aspect of the present invention provides compounds of formula (CIII):

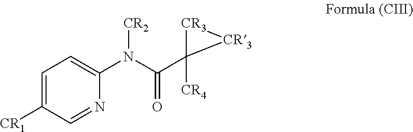

or a pharmaceutically acceptable salt thereof, wherein CR₂, CR₃, CR′₃, and CR₄ are defined in Formula C.

Each CR₁ is aryl or heteroaryl optionally substituted with 1 to 3 of CR^{D}, wherein CR^{D} is —Z^{D}CR₉, wherein each Z^{D} is independently a bond or an optionally substituted branched or straight C₁₋₆ aliphatic chain wherein up to two carbon units of Z^{D} are optionally and independently replaced by —CO—, —CS—, —CONCR^{E}—, —CONCR^{E}NCR^{E}—, —CO₂—, —OCO—, —NCR^{E}CO₂—, —O—, —NCR^{E}CONCR^{E}—, —OCONCR^{E}—, —NCR^{E}NCR^{E}—, —NCR^{E}CO—, —S—, —SO—, —SO₂—, —NCR^{E}—, —SO₂NCR^{E}—, —NCR^{E}SO₂—, or —NCR^{E}SO₂NCR^{E}—; each CR₉ is independently CR^{E}, halo, —OH, —NH₂, —NO₂, —CN, or —OCF₃; each CR^{E} is independently hydrogen, an optionally substituted C₁₋₈ aliphatic group, an optionally substituted cycloaliphatic, an optionally substituted heterocycloaliphatic, an optionally substituted aryl, or an optionally substituted heteroaryl.

In another aspect, the present invention includes compounds of Formula (CIV):

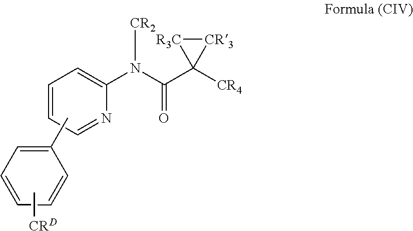

or a pharmaceutically acceptable salt thereof, wherein CR₂, CR₃, CR′₃, and CR₄ are defined in Formula C.

R^{D} is —Z^{D}CR₉, wherein each Z^{D} is independently a bond or an optionally substituted branched or straight C₁₋₆ aliphatic chain wherein up to two carbon units of Z^{D} are optionally and independently replaced by —CO—, —CONCR^{E}—, —CO₂—, —OCO—, —NCR^{E}CO₂—, —O—, —OCONCR^{E}—, —NCR^{E}CO—, —S—, —SO—, —SO₂—, —NCR^{E}—, —SO₂NCR^{E}—, or —NCR^{E}SO₂—.

Each CR₉ is independently CR^{E}, halo, —OH, —NH₂, —NO₂, —CN, or —OCF₃.

Each CR^{E} is independently hydrogen, an optionally substituted C₁₋₈ aliphatic group, an optionally substituted cycloaliphatic, an optionally substituted heterocycloaliphatic, an optionally substituted aryl, or an optionally substituted heteroaryl.

In several embodiments, Z^{D} is independently a bond or an optionally substituted branched or straight C₁₋₆ aliphatic chain wherein one carbon unit of Z^{D} is optionally replaced by —SO₂—, —CONCR^{E}—, or —SO₂NCR^{C}—. For example, Z^{D} is an optionally substituted branched or straight C₁₋₆ aliphatic chain wherein one carbon unit of Z^{D} is optionally replaced by —SO₂—. In other examples, CR₉ is an optionally substituted heteroaryl or an optionally substituted heterocycloaliphatic. In additional examples, CR₉ is an optionally substituted heterocycloaliphatic having 1-2 nitrogen atoms, and CR₉ attaches directly to —SO₂— via a ring nitrogen.

### 6. Exemplary Compounds

Exemplary Column C compounds of the present invention include, but are not limited to, those illustrated in Table II.C-1 below.

TABLE II.C-1
Examples of Column C compounds of the present invention

| | |
|---|---|
| 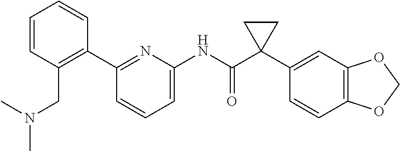 [SMILES: CN(C)Cc1ccccc1-c1cccc(NC(=O)C2(c3ccc4c(c3)OCO4)CC2)n1] | 1 |
| 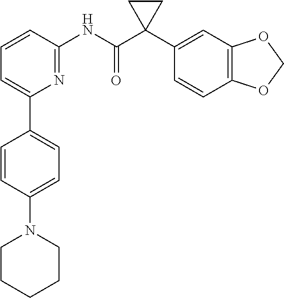 [SMILES: O=C(Nc1cccc(-c2ccc(N3CCCCC3)cc2)n1)C1(c2ccc3c(c2)OCO3)CC1] | 2 |
| 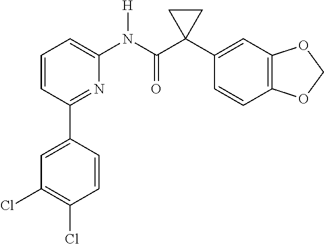 [SMILES: O=C(Nc1cccc(-c2ccc(Cl)c(Cl)c2)n1)C1(c2ccc3c(c2)OCO3)CC1] | 3 |
| 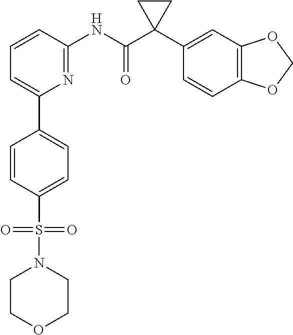 [SMILES: O=C(Nc1cccc(-c2ccc(S(=O)(=O)N3CCOCC3)cc2)n1)C1(c2ccc3c(c2)OCO3)CC1] | 4 |
| 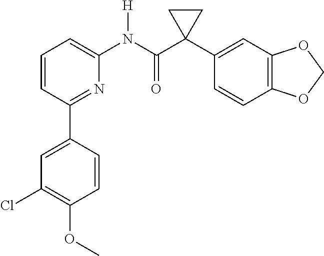 [SMILES: COc1ccc(-c2cccc(NC(=O)C3(c4ccc5c(c4)OCO5)CC3)n2)cc1Cl] | 5 |
| 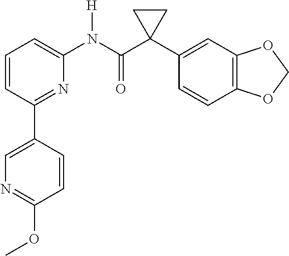 [SMILES: COc1ccc(-c2cccc(NC(=O)C3(c4ccc5c(c4)OCO5)CC3)n2)cn1] | 6 |
| 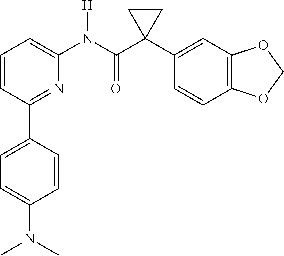 [SMILES: CN(C)c1ccc(-c2cccc(NC(=O)C3(c4ccc5c(c4)OCO5)CC3)n2)cc1] | 7 |
| 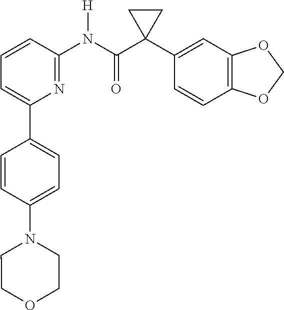 [SMILES: O=C(Nc1cccc(-c2ccc(N3CCOCC3)cc2)n1)C1(c2ccc3c(c2)OCO3)CC1] | 8 |
| 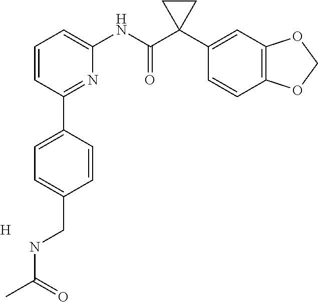 | 9 |
| 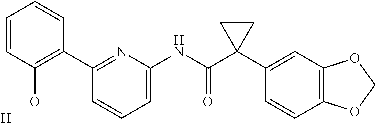 | 10 |
| 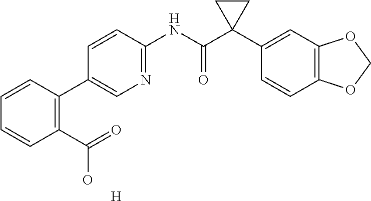 | 11 |
| 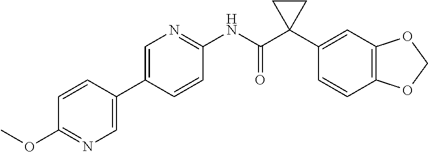 [SMILES: COc1ccc(-c2ccc(NC(=O)C3(c4ccc5c(c4)OCO5)CC3)nc2)cn1] | 12 |
| 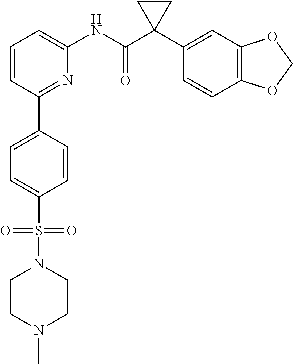 [SMILES: CN1CCN(S(=O)(=O)c2ccc(-c3cccc(NC(=O)C4(c5ccc6c(c5)OCO6)CC4)n3)cc2)CC1] | 13 |
| 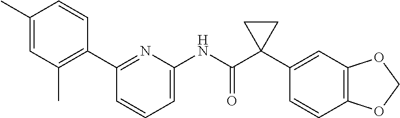 [SMILES: Cc1ccc(-c2cccc(NC(=O)C3(c4ccc5c(c4)OCO5)CC3)n2)c(C)c1] | 14 |
| 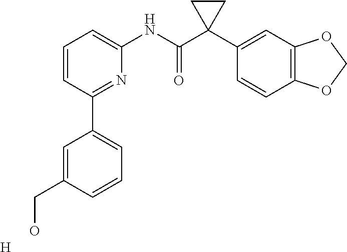 | 15 |
| 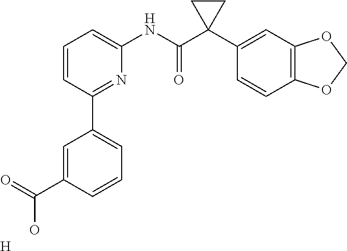 | 16 |
| 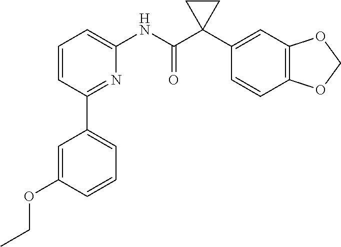 [SMILES: CCOc1cccc(-c2cccc(NC(=O)C3(c4ccc5c(c4)OCO5)CC3)n2)c1] | 17 |
| 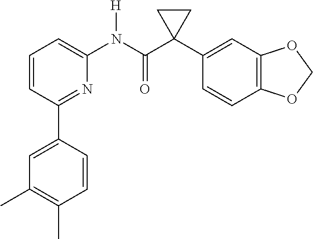 [SMILES: Cc1ccc(-c2cccc(NC(=O)C3(c4ccc5c(c4)OCO5)CC3)n2)cc1C] | 18 |
| 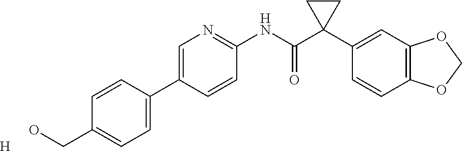 | 19 |
| 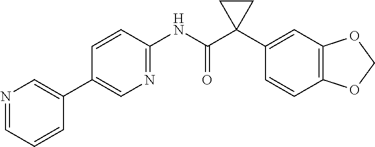 [SMILES: O=C(Nc1ccc(-c2cccnc2)cn1)C1(c2ccc3c(c2)OCO3)CC1] | 20 |
| 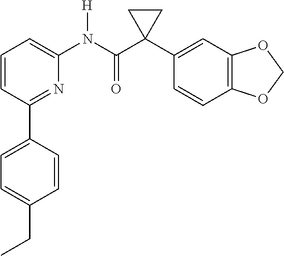 [SMILES: CCc1ccc(-c2cccc(NC(=O)C3(c4ccc5c(c4)OCO5)CC3)n2)cc1] | 21 |
| 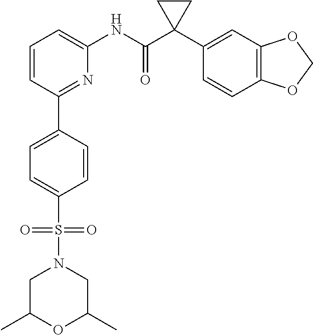 [SMILES: CC1CN(S(=O)(=O)c2ccc(-c3cccc(NC(=O)C4(c5ccc6c(c5)OCO6)CC4)n3)cc2)CC(C)O1] | 22 |
| 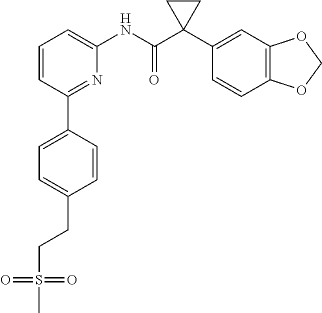 [SMILES: CS(=O)(=O)CCc1ccc(-c2cccc(NC(=O)C3(c4ccc5c(c4)OCO5)CC3)n2)cc1] | 23 |
| 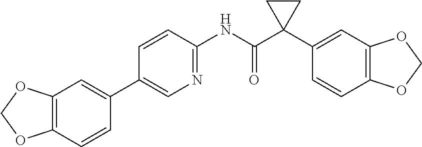 [SMILES: O=C(Nc1ccc(-c2ccc3c(c2)OCO3)cn1)C1(c2ccc3c(c2)OCO3)CC1] | 24 |
| 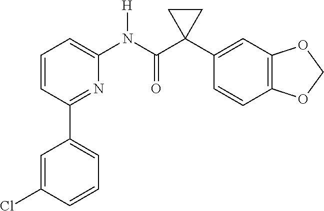 [SMILES: O=C(Nc1cccc(-c2cccc(Cl)c2)n1)C1(c2ccc3c(c2)OCO3)CC1] | 25 |
| 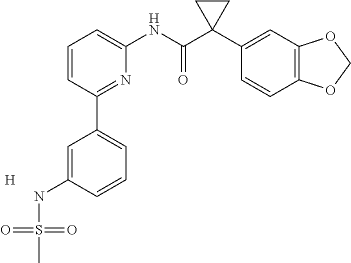 | 26 |
| 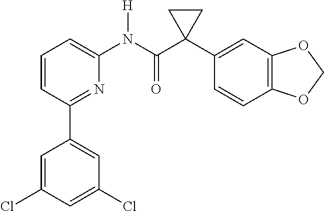 [SMILES: O=C(Nc1cccc(-c2cc(Cl)cc(Cl)c2)n1)C1(c2ccc3c(c2)OCO3)CC1] | 27 |
| 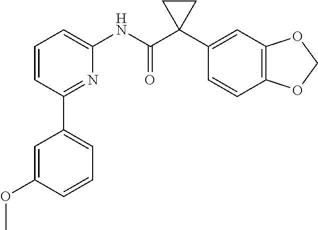 [SMILES: COc1cccc(-c2cccc(NC(=O)C3(c4ccc5c(c4)OCO5)CC3)n2)c1] | 28 |
| 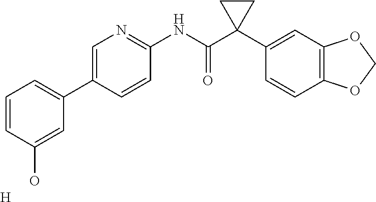 | 29 |
| 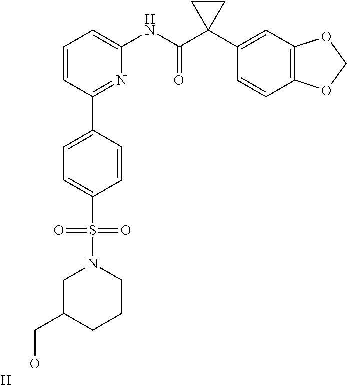 | 30 |
| 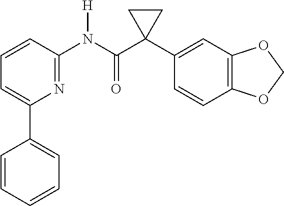 [SMILES: O=C(Nc1cccc(-c2ccccc2)n1)C1(c2ccc3c(c2)OCO3)CC1] | 31 |
| 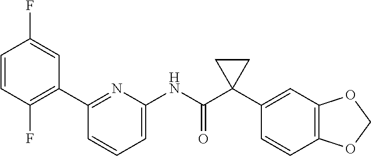 [SMILES: O=C(Nc1cccc(-c2cc(F)ccc2F)n1)C1(c2ccc3c(c2)OCO3)CC1] | 32 |
| 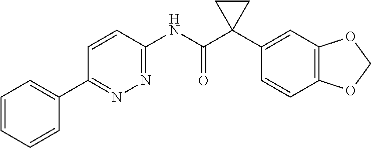 [SMILES: O=C(Nc1ccc(-c2ccccc2)nn1)C1(c2ccc3c(c2)OCO3)CC1] | 33 |
| 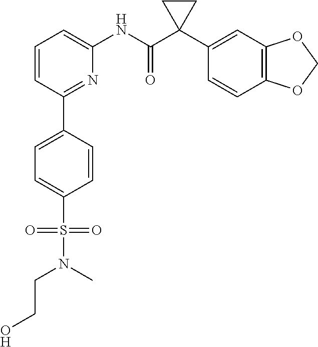 [SMILES: CN(CCO)S(=O)(=O)c1ccc(-c2cccc(NC(=O)C3(c4ccc5c(c4)OCO5)CC3)n2)cc1] | 34 |
| 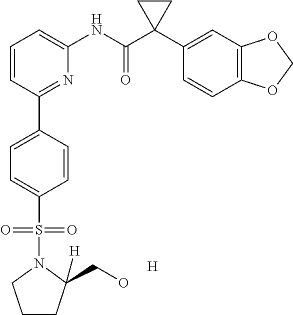 | 35 |
| 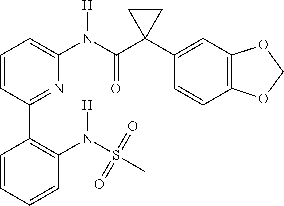 [SMILES: CS(=O)(=O)Nc1ccccc1-c1cccc(NC(=O)C2(c3ccc4c(c3)OCO4)CC2)n1] | 36 |
| 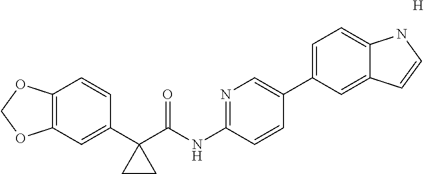 | 37 |
| 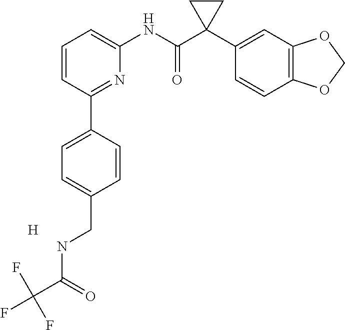 | 38 |
| 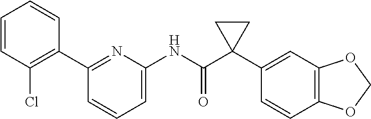 [SMILES: O=C(Nc1cccc(-c2ccccc2Cl)n1)C1(c2ccc3c(c2)OCO3)CC1] | 39 |
| 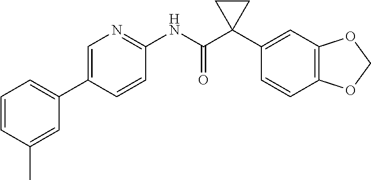 [SMILES: Cc1cccc(-c2ccc(NC(=O)C3(c4ccc5c(c4)OCO5)CC3)nc2)c1] | 40 |
| 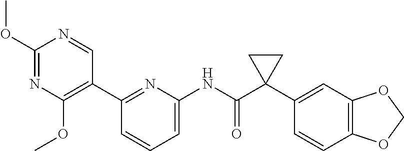 [SMILES: COc1ncc(-c2cccc(NC(=O)C3(c4ccc5c(c4)OCO5)CC3)n2)c(OC)n1] | 41 |
| 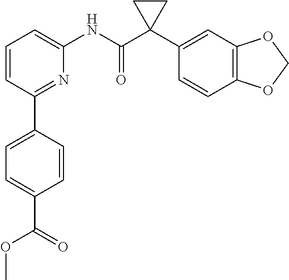 [SMILES: COC(=O)c1ccc(-c2cccc(NC(=O)C3(c4ccc5c(c4)OCO5)CC3)n2)cc1] | 42 |
| 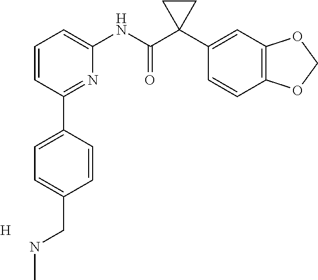 | 43 |
| 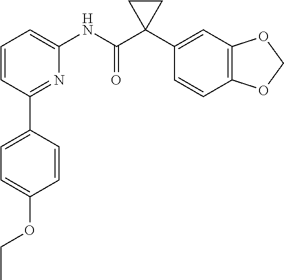 [SMILES: CCOc1ccc(-c2cccc(NC(=O)C3(c4ccc5c(c4)OCO5)CC3)n2)cc1] | 44 |
| 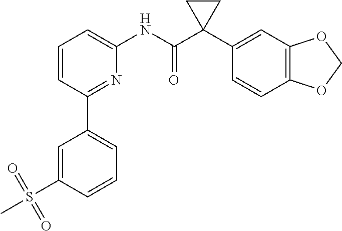 [SMILES: CS(=O)(=O)c1cccc(-c2cccc(NC(=O)C3(c4ccc5c(c4)OCO5)CC3)n2)c1] | 45 |
| 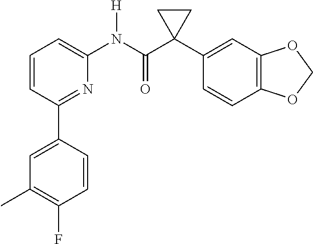 [SMILES: Cc1cc(-c2cccc(NC(=O)C3(c4ccc5c(c4)OCO5)CC3)n2)ccc1F] | 46 |
| 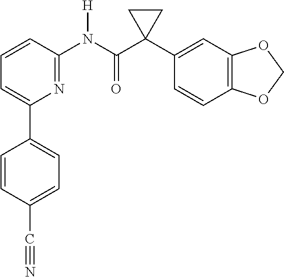 [SMILES: N#Cc1ccc(-c2cccc(NC(=O)C3(c4ccc5c(c4)OCO5)CC3)n2)cc1] | 47 |
| 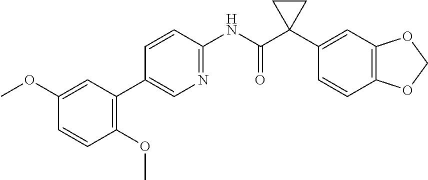 [SMILES: COc1ccc(OC)c(-c2ccc(NC(=O)C3(c4ccc5c(c4)OCO5)CC3)nc2)c1] | 48 |
| 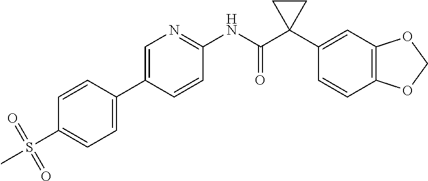 [SMILES: CS(=O)(=O)c1ccc(-c2ccc(NC(=O)C3(c4ccc5c(c4)OCO5)CC3)nc2)cc1] | 49 |
| 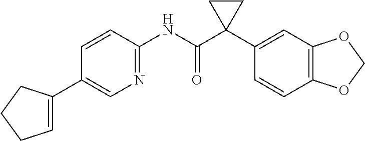 [SMILES: O=C(Nc1ccc(C2=CCCC2)cn1)C1(c2ccc3c(c2)OCO3)CC1] | 50 |
| 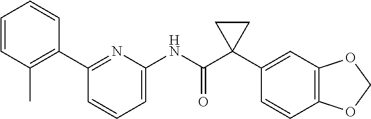 [SMILES: Cc1ccccc1-c1cccc(NC(=O)C2(c3ccc4c(c3)OCO4)CC2)n1] | 51 |
| 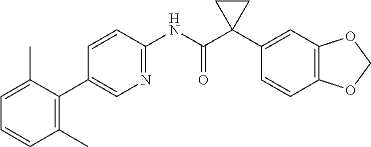 [SMILES: Cc1cccc(C)c1-c1ccc(NC(=O)C2(c3ccc4c(c3)OCO4)CC2)nc1] | 52 |
| 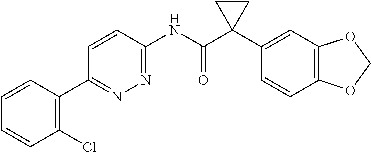 [SMILES: O=C(Nc1ccc(-c2ccccc2Cl)nn1)C1(c2ccc3c(c2)OCO3)CC1] | 53 |
| 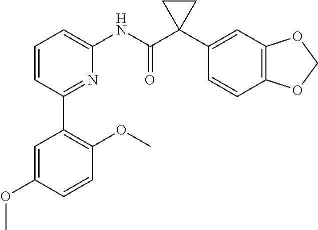 [SMILES: COc1ccc(OC)c(-c2cccc(NC(=O)C3(c4ccc5c(c4)OCO5)CC3)n2)c1] | 54 |
| 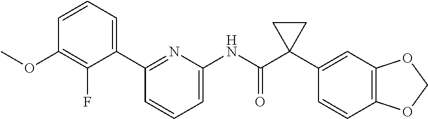 [SMILES: COc1cccc(-c2cccc(NC(=O)C3(c4ccc5c(c4)OCO5)CC3)n2)c1F] | 55 |
| 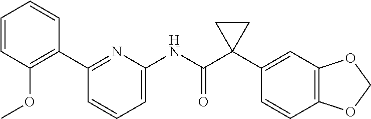 [SMILES: COc1ccccc1-c1cccc(NC(=O)C2(c3ccc4c(c3)OCO4)CC2)n1] | 56 |
| 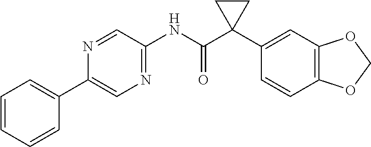 [SMILES: O=C(Nc1cnc(-c2ccccc2)cn1)C1(c2ccc3c(c2)OCO3)CC1] | 57 |
| 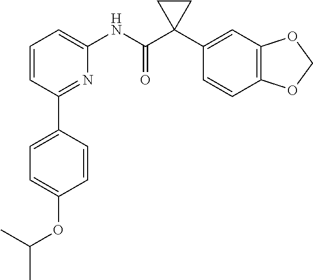 [SMILES: CC(C)Oc1ccc(-c2cccc(NC(=O)C3(c4ccc5c(c4)OCO5)CC3)n2)cc1] | 58 |
| 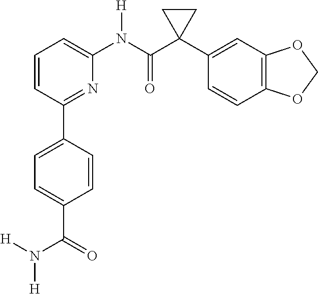 [SMILES: NC(=O)c1ccc(-c2cccc(NC(=O)C3(c4ccc5c(c4)OCO5)CC3)n2)cc1] | 59 |
| 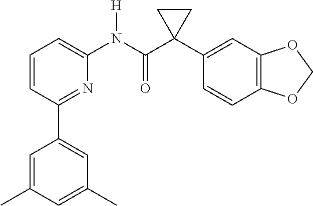 [SMILES: Cc1cc(C)cc(-c2cccc(NC(=O)C3(c4ccc5c(c4)OCO5)CC3)n2)c1] | 60 |
| 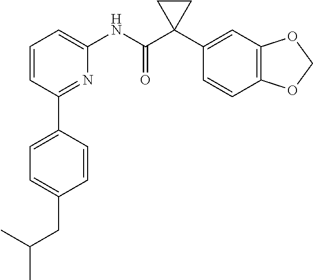 [SMILES: CC(C)Cc1ccc(-c2cccc(NC(=O)C3(c4ccc5c(c4)OCO5)CC3)n2)cc1] | 61 |
| 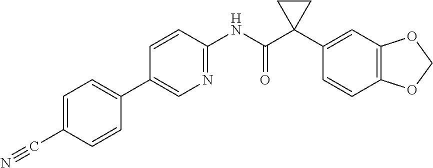 [SMILES: N#Cc1ccc(-c2ccc(NC(=O)C3(c4ccc5c(c4)OCO5)CC3)nc2)cc1] | 62 |
| 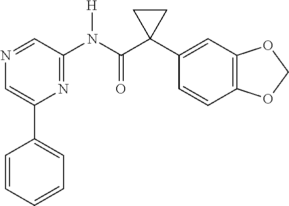 [SMILES: O=C(Nc1cncc(-c2ccccc2)n1)C1(c2ccc3c(c2)OCO3)CC1] | 63 |
| 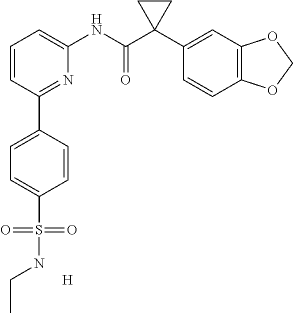 | 64 |
| 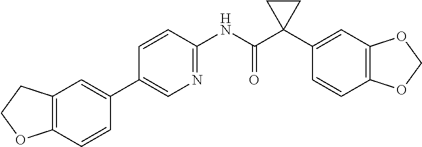 [SMILES: O=C(Nc1ccc(-c2ccc3c(c2)CCO3)cn1)C1(c2ccc3c(c2)OCO3)CC1] | 65 |
| 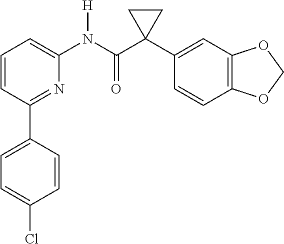 [SMILES: O=C(Nc1cccc(-c2ccc(Cl)cc2)n1)C1(c2ccc3c(c2)OCO3)CC1] | 66 |
| 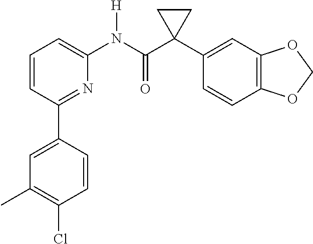 [SMILES: Cc1cc(-c2cccc(NC(=O)C3(c4ccc5c(c4)OCO5)CC3)n2)ccc1Cl] | 67 |
| 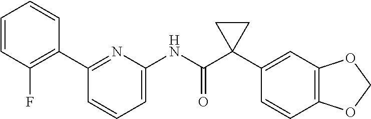 [SMILES: O=C(Nc1cccc(-c2ccccc2F)n1)C1(c2ccc3c(c2)OCO3)CC1] | 68 |
| 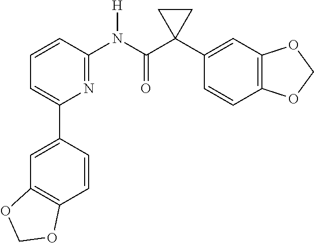 [SMILES: O=C(Nc1cccc(-c2ccc3c(c2)OCO3)n1)C1(c2ccc3c(c2)OCO3)CC1] | 69 |
| 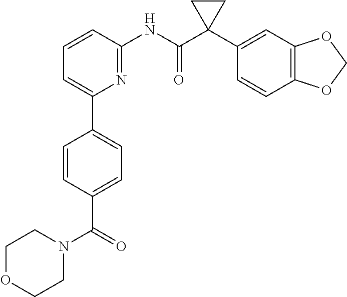 [SMILES: O=C(c1ccc(-c2cccc(NC(=O)C3(c4ccc5c(c4)OCO5)CC3)n2)cc1)N1CCOCC1] | 70 |
| 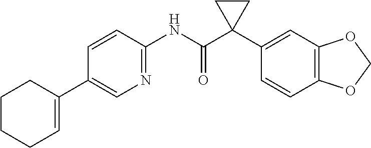 [SMILES: O=C(Nc1ccc(C2=CCCCC2)cn1)C1(c2ccc3c(c2)OCO3)CC1] | 71 |
| 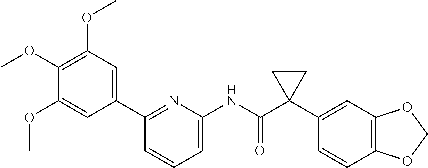 [SMILES: COc1cc(-c2cccc(NC(=O)C3(c4ccc5c(c4)OCO5)CC3)n2)cc(OC)c1OC] | 72 |
| 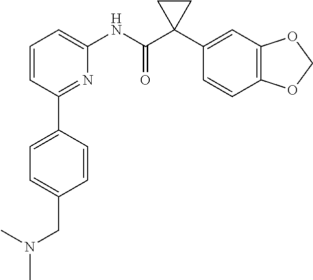 [SMILES: CN(C)Cc1ccc(-c2cccc(NC(=O)C3(c4ccc5c(c4)OCO5)CC3)n2)cc1] | 73 |
| 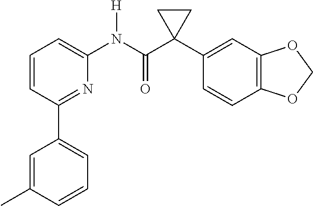 [SMILES: Cc1cccc(-c2cccc(NC(=O)C3(c4ccc5c(c4)OCO5)CC3)n2)c1] | 74 |
| 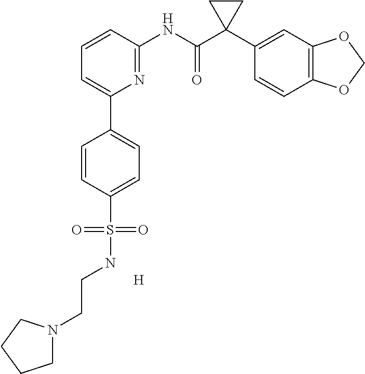 | 75 |
| 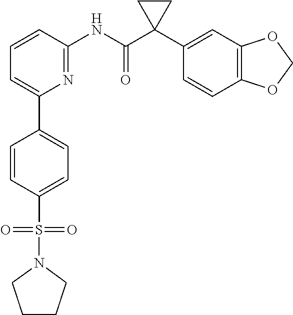 [SMILES: O=C(Nc1cccc(-c2ccc(S(=O)(=O)N3CCCC3)cc2)n1)C1(c2ccc3c(c2)OCO3)CC1] | 76 |
| 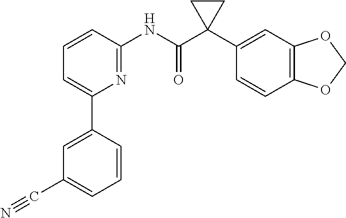 [SMILES: N#Cc1cccc(-c2cccc(NC(=O)C3(c4ccc5c(c4)OCO5)CC3)n2)c1] | 77 |
| 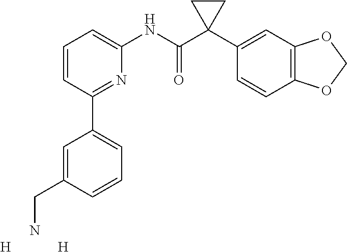 | 78 |
| 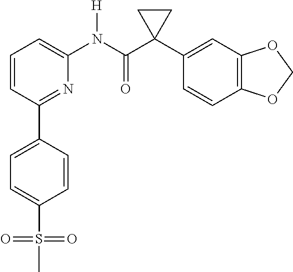 [SMILES: CS(=O)(=O)c1ccc(-c2cccc(NC(=O)C3(c4ccc5c(c4)OCO5)CC3)n2)cc1] | 79 |
| 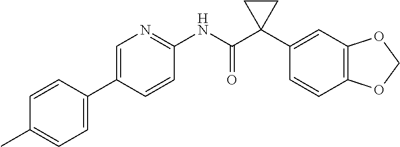 [SMILES: Cc1ccc(-c2ccc(NC(=O)C3(c4ccc5c(c4)OCO5)CC3)nc2)cc1] | 80 |
| 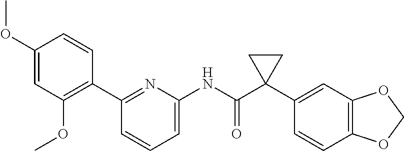 [SMILES: COc1ccc(-c2cccc(NC(=O)C3(c4ccc5c(c4)OCO5)CC3)n2)c(OC)c1] | 81 |
| 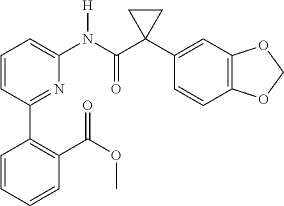 [SMILES: COC(=O)c1ccccc1-c1cccc(NC(=O)C2(c3ccc4c(c3)OCO4)CC2)n1] | 82 |
| 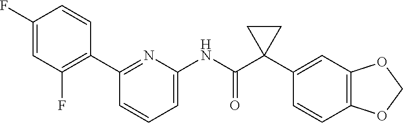 [SMILES: O=C(Nc1cccc(-c2ccc(F)cc2F)n1)C1(c2ccc3c(c2)OCO3)CC1] | 83 |
| 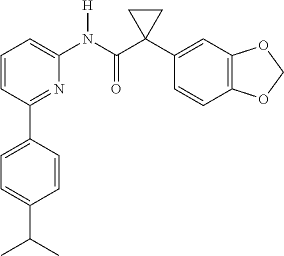 [SMILES: CC(C)c1ccc(-c2cccc(NC(=O)C3(c4ccc5c(c4)OCO5)CC3)n2)cc1] | 84 |
| 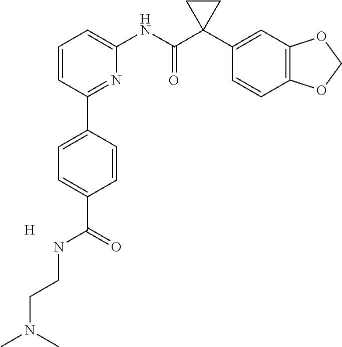 | 85 |
| 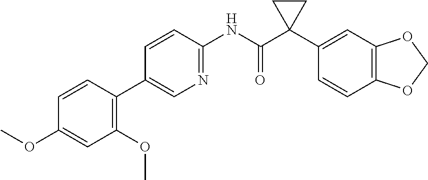 [SMILES: COc1ccc(-c2ccc(NC(=O)C3(c4ccc5c(c4)OCO5)CC3)nc2)c(OC)c1] | 86 |
| 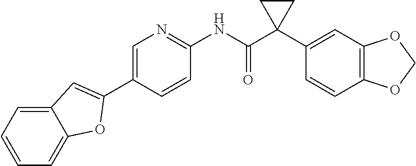 [SMILES: O=C(Nc1ccc(-c2cc3ccccc3o2)cn1)C1(c2ccc3c(c2)OCO3)CC1] | 87 |
| 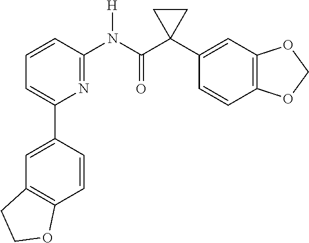 [SMILES: O=C(Nc1cccc(-c2ccc3c(c2)CCO3)n1)C1(c2ccc3c(c2)OCO3)CC1] | 88 |
| 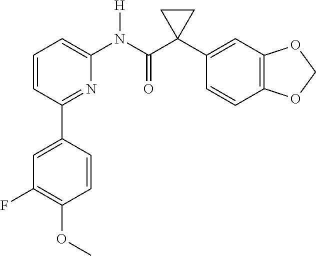 [SMILES: COc1ccc(-c2cccc(NC(=O)C3(c4ccc5c(c4)OCO5)CC3)n2)cc1F] | 89 |
| 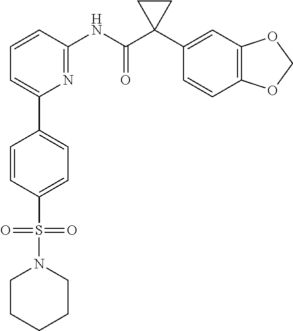 [SMILES: O=C(Nc1cccc(-c2ccc(S(=O)(=O)N3CCCCC3)cc2)n1)C1(c2ccc3c(c2)OCO3)CC1] | 90 |
| 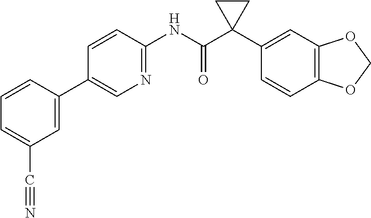 [SMILES: N#Cc1cccc(-c2ccc(NC(=O)C3(c4ccc5c(c4)OCO5)CC3)nc2)c1] | 91 |
| 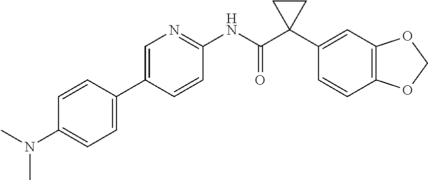 [SMILES: CN(C)c1ccc(-c2ccc(NC(=O)C3(c4ccc5c(c4)OCO5)CC3)nc2)cc1] | 92 |
| 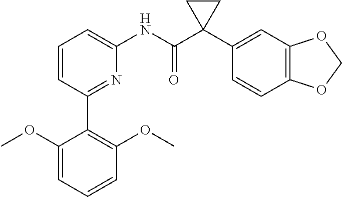 [SMILES: COc1cccc(OC)c1-c1cccc(NC(=O)C2(c3ccc4c(c3)OCO4)CC2)n1] | 93 |
| 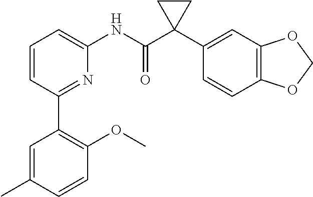 [SMILES: COc1ccc(C)cc1-c1cccc(NC(=O)C2(c3ccc4c(c3)OCO4)CC2)n1] | 94 |
| 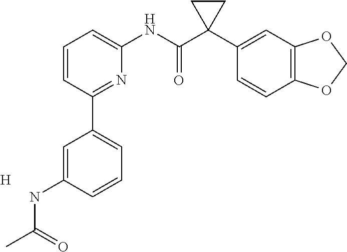 | 95 |
| 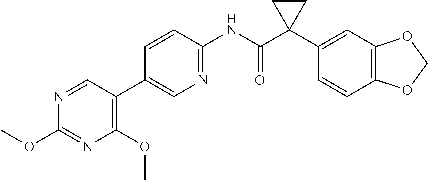 [SMILES: COc1ncc(-c2ccc(NC(=O)C3(c4ccc5c(c4)OCO5)CC3)nc2)c(OC)n1] | 96 |
| 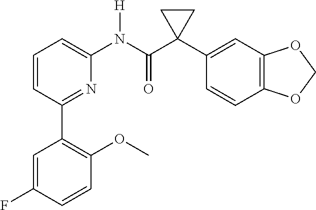 [SMILES: COc1ccc(F)cc1-c1cccc(NC(=O)C2(c3ccc4c(c3)OCO4)CC2)n1] | 97 |
| 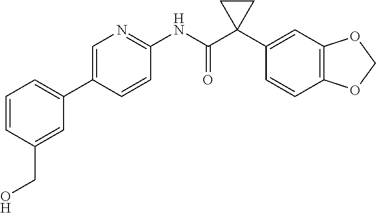 [SMILES: O=C(Nc1ccc(-c2cccc(CO)c2)cn1)C1(c2ccc3c(c2)OCO3)CC1] | 98 |
| 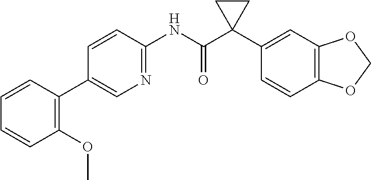 [SMILES: COc1ccccc1-c1ccc(NC(=O)C2(c3ccc4c(c3)OCO4)CC2)nc1] | 99 |
| 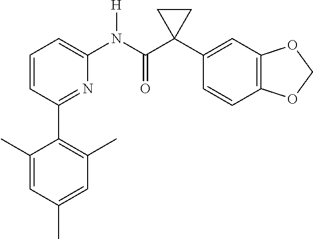 [SMILES: Cc1cc(C)c(-c2cccc(NC(=O)C3(c4ccc5c(c4)OCO5)CC3)n2)c(C)c1] | 100 |
| 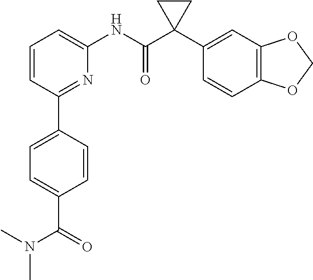 [SMILES: CN(C)C(=O)c1ccc(-c2cccc(NC(=O)C3(c4ccc5c(c4)OCO5)CC3)n2)cc1] | 101 |
| 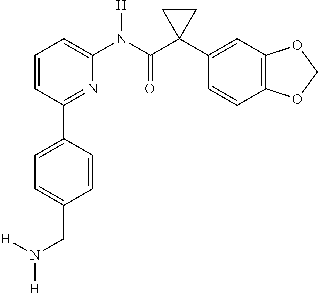 [SMILES: NCc1ccc(-c2cccc(NC(=O)C3(c4ccc5c(c4)OCO5)CC3)n2)cc1] | 102 |
| 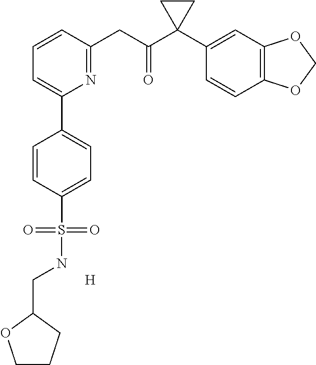 | 103 |
| 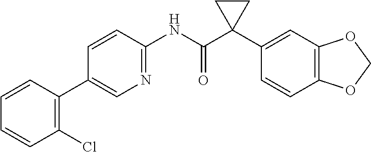 [SMILES: O=C(Nc1ccc(-c2ccccc2Cl)cn1)C1(c2ccc3c(c2)OCO3)CC1] | 104 |
| 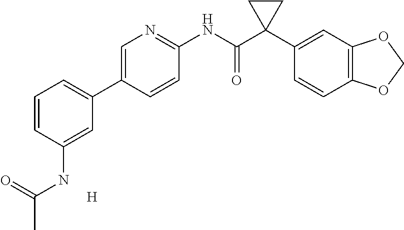 | 105 |
| 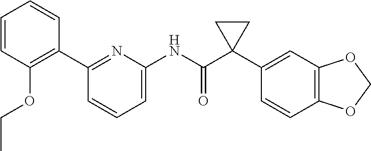 [SMILES: CCOc1ccccc1-c1cccc(NC(=O)C2(c3ccc4c(c3)OCO4)CC2)n1] | 106 |
| 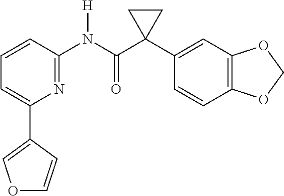 [SMILES: O=C(Nc1cccc(-c2ccoc2)n1)C1(c2ccc3c(c2)OCO3)CC1] | 107 |
| 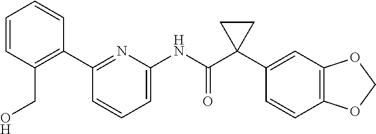 [SMILES: O=C(Nc1cccc(-c2ccccc2CO)n1)C1(c2ccc3c(c2)OCO3)CC1] | 108 |
| 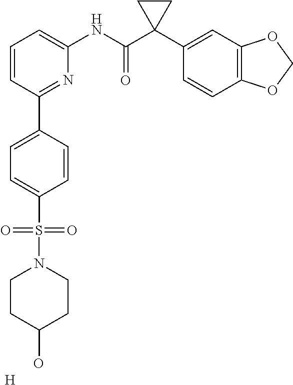 | 109 |
| 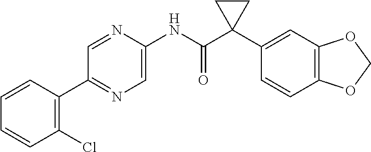 [SMILES: O=C(Nc1cnc(-c2ccccc2Cl)cn1)C1(c2ccc3c(c2)OCO3)CC1] | 110 |
| 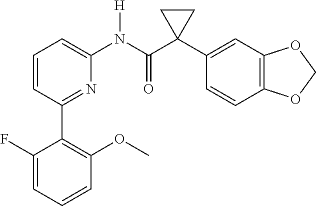 [SMILES: COc1cccc(F)c1-c1cccc(NC(=O)C2(c3ccc4c(c3)OCO4)CC2)n1] | 111 |
| 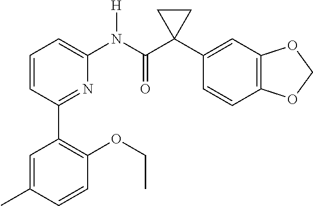 [SMILES: CCOc1ccc(C)cc1-c1cccc(NC(=O)C2(c3ccc4c(c3)OCO4)CC2)n1] | 112 |
| 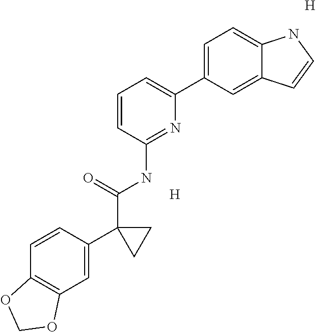 | 113 |
| 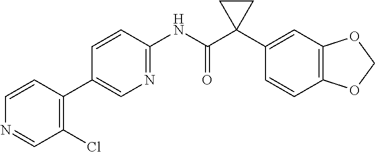 [SMILES: O=C(Nc1ccc(-c2ccncc2Cl)cn1)C1(c2ccc3c(c2)OCO3)CC1] | 114 |
| 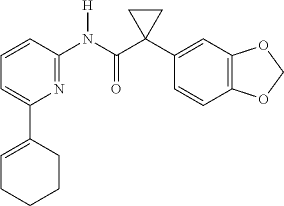 [SMILES: O=C(Nc1cccc(C2=CCCCC2)n1)C1(c2ccc3c(c2)OCO3)CC1] | 115 |
| 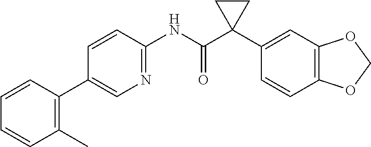 [SMILES: Cc1ccccc1-c1ccc(NC(=O)C2(c3ccc4c(c3)OCO4)CC2)nc1] | 116 |
| 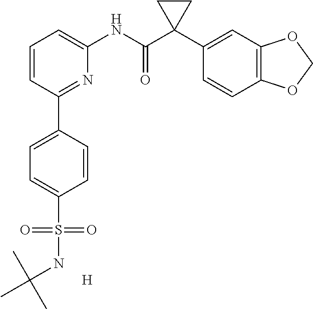 | 117 |
| 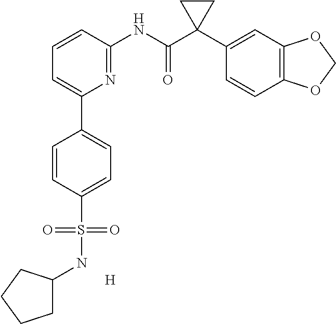 | 118 |
| 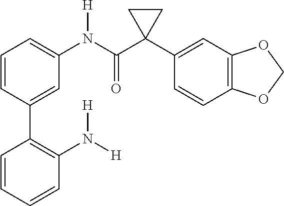 [SMILES: Nc1ccccc1-c1cccc(NC(=O)C2(c3ccc4c(c3)OCO4)CC2)c1] | 119 |
| 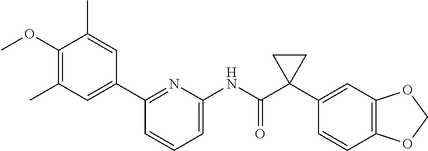 [SMILES: COc1c(C)cc(-c2cccc(NC(=O)C3(c4ccc5c(c4)OCO5)CC3)n2)cc1C] | 120 |
| 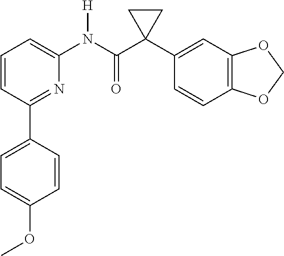 [SMILES: COc1ccc(-c2cccc(NC(=O)C3(c4ccc5c(c4)OCO5)CC3)n2)cc1] | 121 |
| 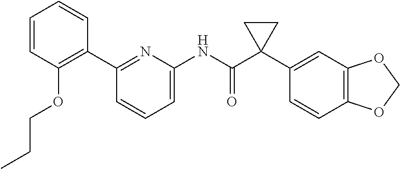 [SMILES: CCCOc1ccccc1-c1cccc(NC(=O)C2(c3ccc4c(c3)OCO4)CC2)n1] | 122 |
| 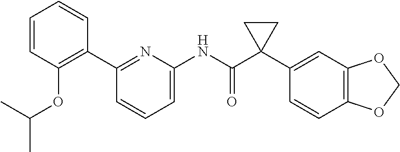 [SMILES: CC(C)Oc1ccccc1-c1cccc(NC(=O)C2(c3ccc4c(c3)OCO4)CC2)n1] | 123 |
| 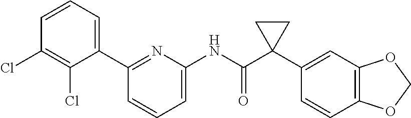 [SMILES: O=C(Nc1cccc(-c2cccc(Cl)c2Cl)n1)C1(c2ccc3c(c2)OCO3)CC1] | 124 |
| 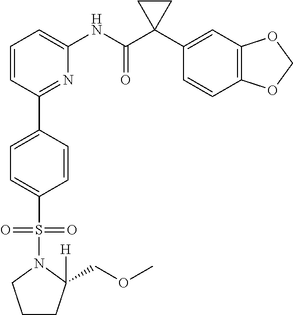 [SMILES: COC[C@@H]1CCCN1S(=O)(=O)c1ccc(-c2cccc(NC(=O)C3(c4ccc5c(c4)OCO5)CC3)n2)cc1] | 125 |
| 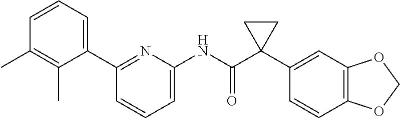 [SMILES: Cc1cccc(-c2cccc(NC(=O)C3(c4ccc5c(c4)OCO5)CC3)n2)c1C] | 126 |
| 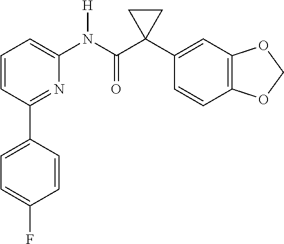 [SMILES: O=C(Nc1cccc(-c2ccc(F)cc2)n1)C1(c2ccc3c(c2)OCO3)CC1] | 127 |
| 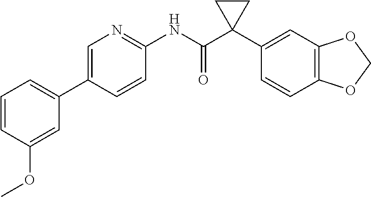 [SMILES: COc1cccc(-c2ccc(NC(=O)C3(c4ccc5c(c4)OCO5)CC3)nc2)c1] | 128 |
| 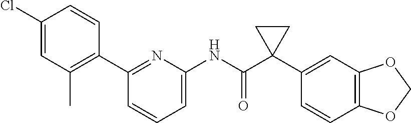 [SMILES: Cc1cc(Cl)ccc1-c1cccc(NC(=O)C2(c3ccc4c(c3)OCO4)CC2)n1] | 129 |
| 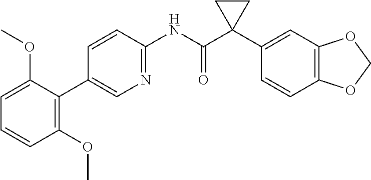 [SMILES: COc1cccc(OC)c1-c1ccc(NC(=O)C2(c3ccc4c(c3)OCO4)CC2)nc1] | 130 |
| 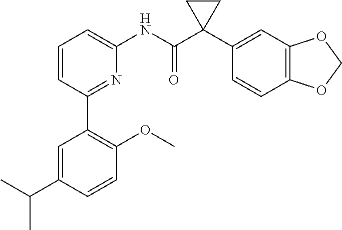 [SMILES: COc1ccc(C(C)C)cc1-c1cccc(NC(=O)C2(c3ccc4c(c3)OCO4)CC2)n1] | 131 |
| 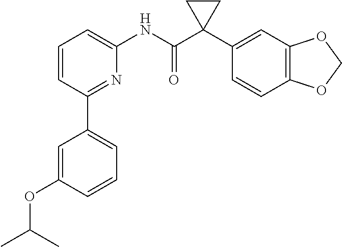 [SMILES: CC(C)Oc1cccc(-c2cccc(NC(=O)C3(c4ccc5c(c4)OCO5)CC3)n2)c1] | 132 |
| 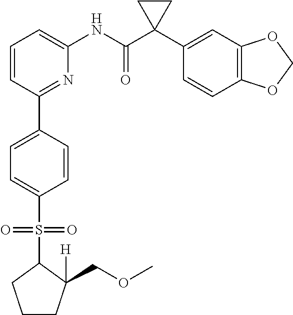 [SMILES: COC[C@H]1CCCC1S(=O)(=O)c1ccc(-c2cccc(NC(=O)C3(c4ccc5c(c4)OCO5)CC3)n2)cc1] | 133 |
| 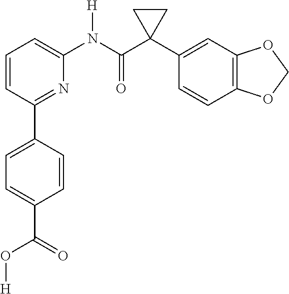 [SMILES: O=C(O)c1ccc(-c2cccc(NC(=O)C3(c4ccc5c(c4)OCO5)CC3)n2)cc1] | 134 |
| 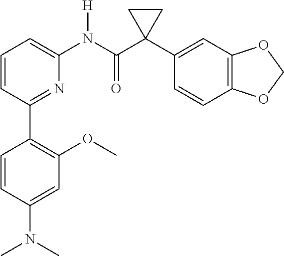 [SMILES: COc1cc(N(C)C)ccc1-c1cccc(NC(=O)C2(c3ccc4c(c3)OCO4)CC2)n1] | 135 |
| 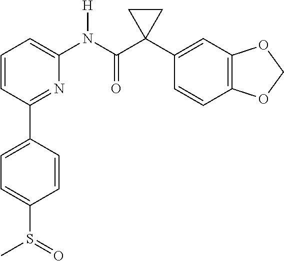 [SMILES: CS(=O)c1ccc(-c2cccc(NC(=O)C3(c4ccc5c(c4)OCO5)CC3)n2)cc1] | 136 |
| 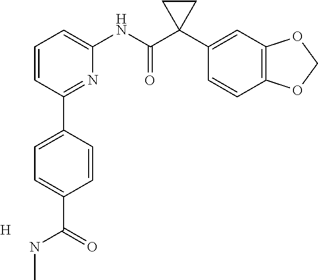 | 137 |
| 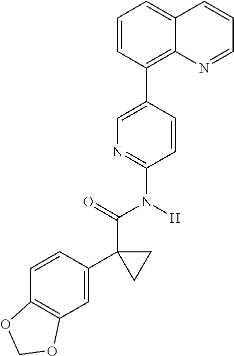 [SMILES: O=C(Nc1ccc(-c2cccc3cccnc23)cn1)C1(c2ccc3c(c2)OCO3)CC1] | 138 |
| 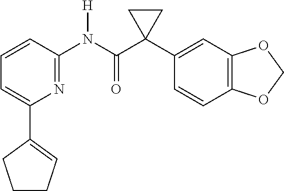 [SMILES: O=C(Nc1cccc(C2=CCCC2)n1)C1(c2ccc3c(c2)OCO3)CC1] | 139 |
| 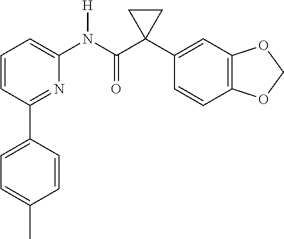 [SMILES: Cc1ccc(-c2cccc(NC(=O)C3(c4ccc5c(c4)OCO5)CC3)n2)cc1] | 140 |
| 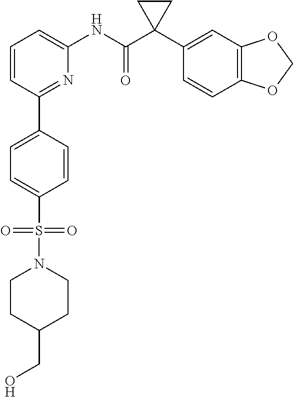 [SMILES: O=C(Nc1cccc(-c2ccc(S(=O)(=O)N3CCC(CO)CC3)cc2)n1)C1(c2ccc3c(c2)OCO3)CC1] | 141 |
| 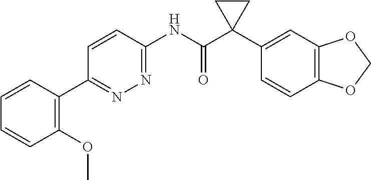 [SMILES: COc1ccccc1-c1ccc(NC(=O)C2(c3ccc4c(c3)OCO4)CC2)nn1] | 142 |
| 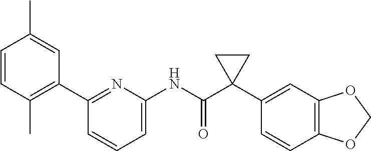 [SMILES: Cc1ccc(C)c(-c2cccc(NC(=O)C3(c4ccc5c(c4)OCO5)CC3)n2)c1] | 143 |
| 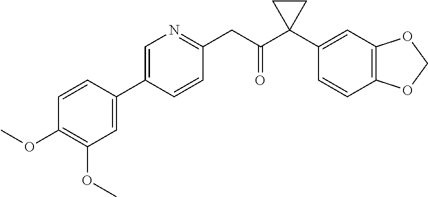 [SMILES: COc1ccc(-c2ccc(CC(=O)C3(c4ccc5c(c4)OCO5)CC3)nc2)cc1OC] | 144 |
| 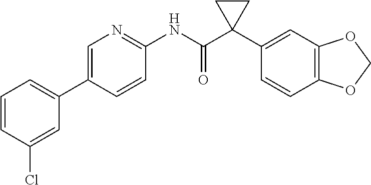 [SMILES: O=C(Nc1ccc(-c2cccc(Cl)c2)cn1)C1(c2ccc3c(c2)OCO3)CC1] | 145 |
| 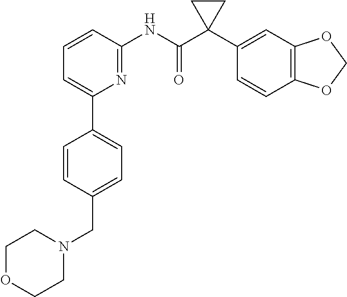 [SMILES: O=C(Nc1cccc(-c2ccc(CN3CCOCC3)cc2)n1)C1(c2ccc3c(c2)OCO3)CC1] | 146 |
| 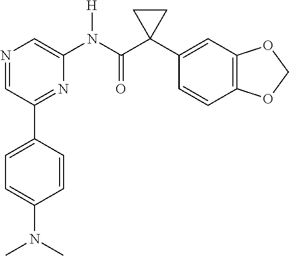 [SMILES: CN(C)c1ccc(-c2cncc(NC(=O)C3(c4ccc5c(c4)OCO5)CC3)n2)cc1] | 147 |
| 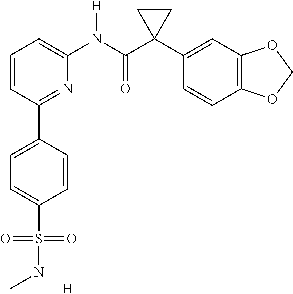 | 148 |
| 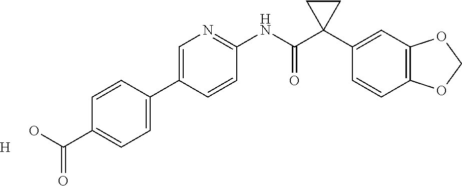 | 149 |
| 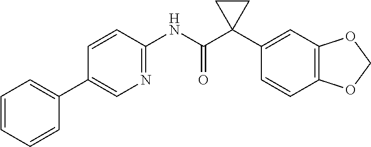 [SMILES: O=C(Nc1ccc(-c2ccccc2)cn1)C1(c2ccc3c(c2)OCO3)CC1] | 150 |
| 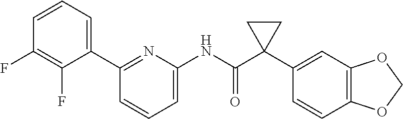 [SMILES: O=C(Nc1cccc(-c2cccc(F)c2F)n1)C1(c2ccc3c(c2)OCO3)CC1] | 151 |
| 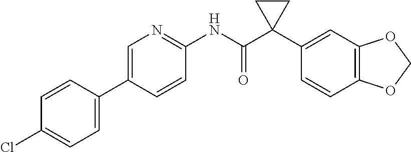 [SMILES: O=C(Nc1ccc(-c2ccc(Cl)cc2)cn1)C1(c2ccc3c(c2)OCO3)CC1] | 152 |
| 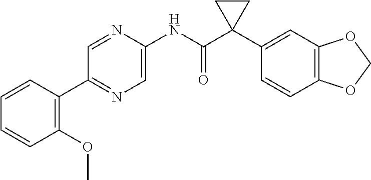 [SMILES: COc1ccccc1-c1cnc(NC(=O)C2(c3ccc4c(c3)OCO4)CC2)cn1] | 153 |
| 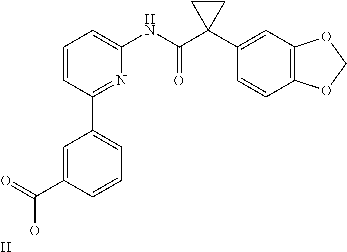 | 154 |
| 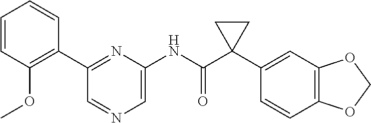 [SMILES: COc1ccccc1-c1cncc(NC(=O)C2(c3ccc4c(c3)OCO4)CC2)n1] | 155 |
| 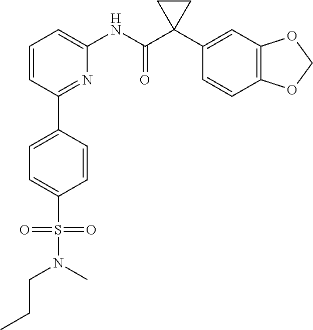 [SMILES: CCCN(C)S(=O)(=O)c1ccc(-c2cccc(NC(=O)C3(c4ccc5c(c4)OCO5)CC3)n2)cc1] | 156 |
| 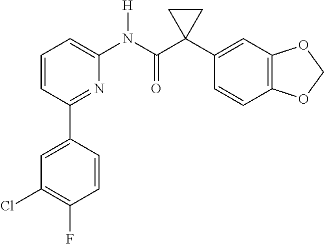 [SMILES: O=C(Nc1cccc(-c2ccc(F)c(Cl)c2)n1)C1(c2ccc3c(c2)OCO3)CC1] | 157 |
| 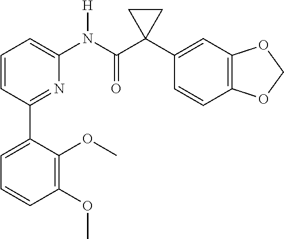 [SMILES: COc1cccc(-c2cccc(NC(=O)C3(c4ccc5c(c4)OCO5)CC3)n2)c1OC] | 158 |
| 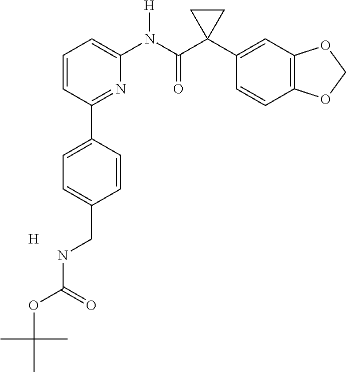 | 159 |
| 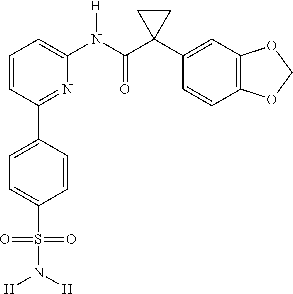 [SMILES: NS(=O)(=O)c1ccc(-c2cccc(NC(=O)C3(c4ccc5c(c4)OCO5)CC3)n2)cc1] | 160 |
| 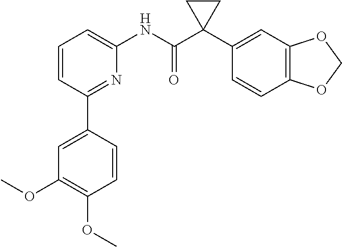 [SMILES: COc1ccc(-c2cccc(NC(=O)C3(c4ccc5c(c4)OCO5)CC3)n2)cc1OC] | 161 |
| 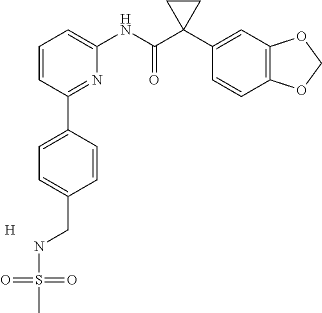 | 162 |
| 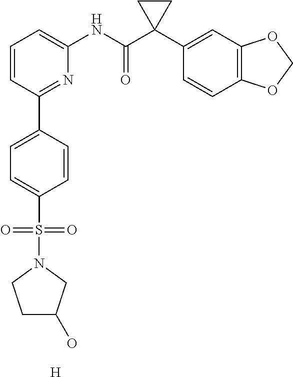 | 163 |
| 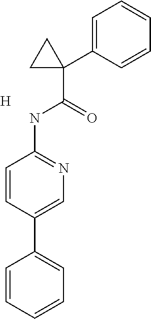 | 164 |
| 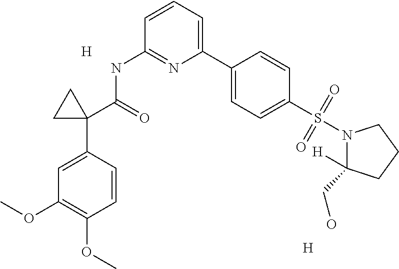 | 165 |
| 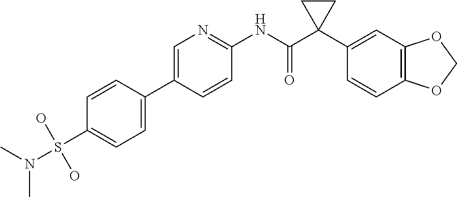 [SMILES: CN(C)S(O)(O)c1ccc(-c2ccc(NC(=O)C3(c4ccc5c(c4)OCO5)CC3)nc2)cc1] | 166 |
| 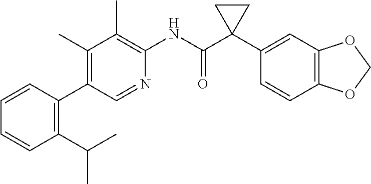 [SMILES: Cc1c(-c2ccccc2C(C)C)cnc(NC(=O)C2(c3ccc4c(c3)OCO4)CC2)c1C] | 167 |
| 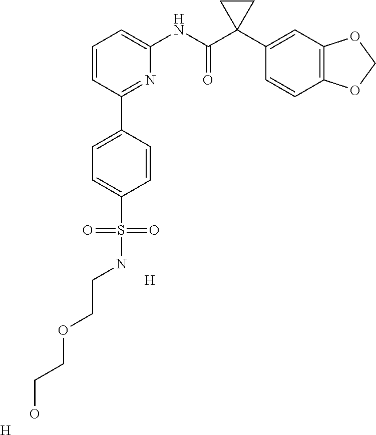 | 168 |
| 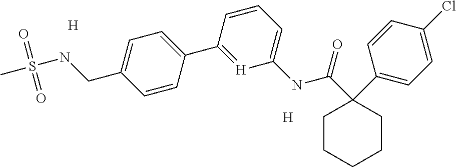 | 169 |
| 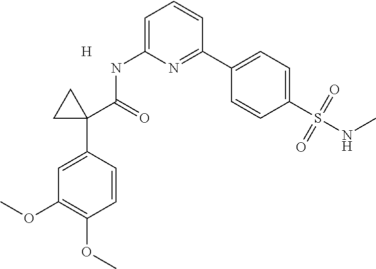 | 170 |
| 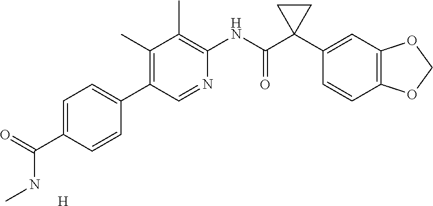 | 171 |
| 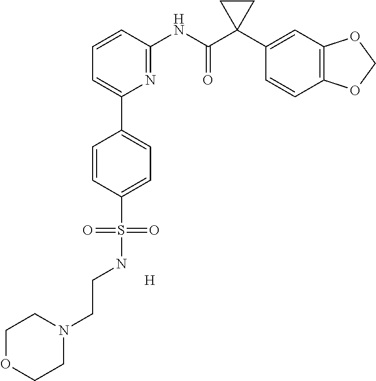 | 172 |
| 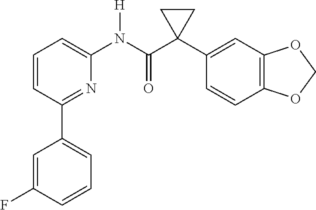 [SMILES: O=C(Nc1cccc(-c2cccc(F)c2)n1)C1(c2ccc3c(c2)OCO3)CC1] | 173 |
| 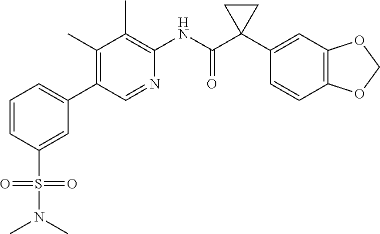 [SMILES: Cc1c(-c2cccc(S(=O)(=O)N(C)C)c2)cnc(NC(=O)C2(c3ccc4c(c3)OCO4)CC2)c1C] | 174 |
| 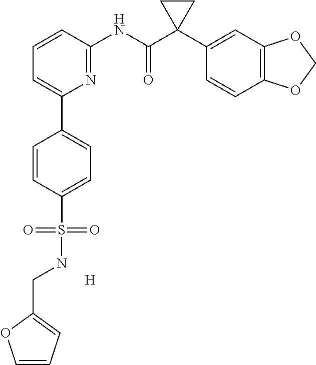 | 175 |
| 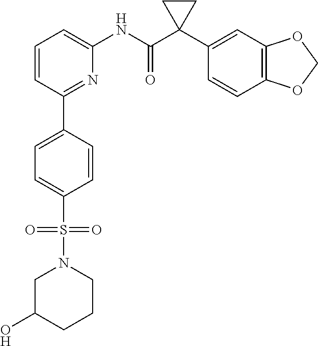 [SMILES: O=C(Nc1cccc(-c2ccc(S(=O)(=O)N3CCCC(O)C3)cc2)n1)C1(c2ccc3c(c2)OCO3)CC1] | 176 |
| 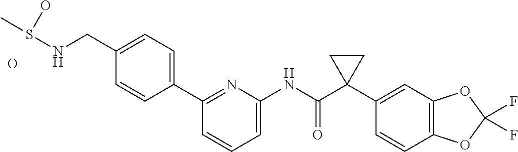 | 177 |
| 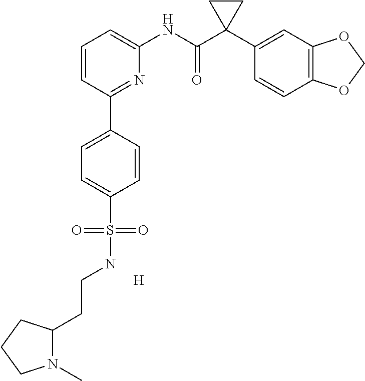 | 178 |
| 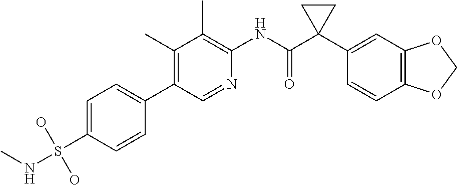 [SMILES: CNS(O)(O)c1ccc(-c2cnc(NC(=O)C3(c4ccc5c(c4)OCO5)CC3)c(C)c2C)cc1] | 179 |
| 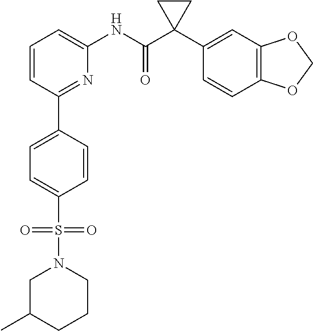 [SMILES: CC1CCCN(S(=O)(=O)c2ccc(-c3cccc(NC(=O)C4(c5ccc6c(c5)OCO6)CC4)n3)cc2)C1] | 180 |
| 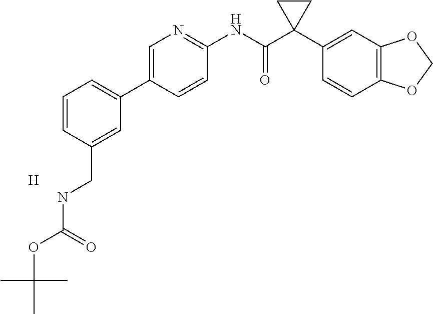 | 181 |
| 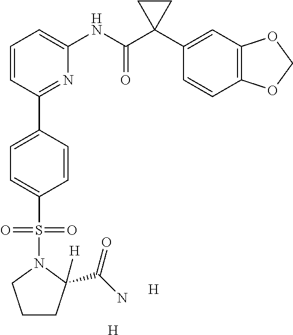 | 182 |
| 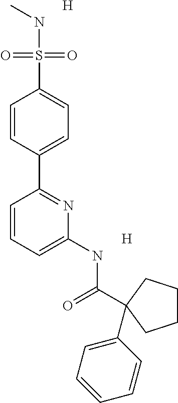 | 183 |
| 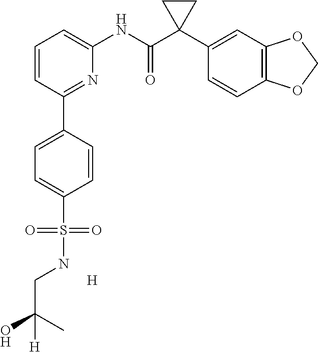 | 184 |
| 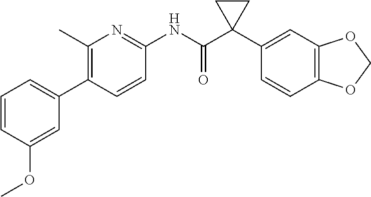 [SMILES: COc1cccc(-c2ccc(NC(=O)C3(c4ccc5c(c4)OCO5)CC3)nc2C)c1] | 185 |
| 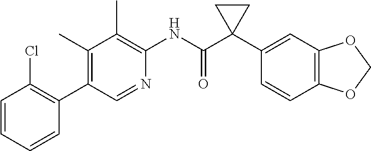 [SMILES: Cc1c(-c2ccccc2Cl)cnc(NC(=O)C2(c3ccc4c(c3)OCO4)CC2)c1C] | 186 |
| 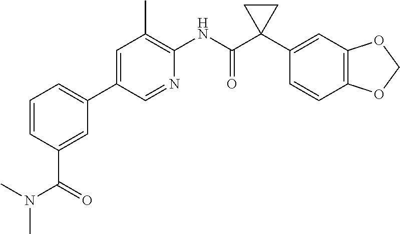 [SMILES: Cc1cc(-c2cccc(C(=O)N(C)C)c2)cnc1NC(=O)C1(c2ccc3c(c2)OCO3)CC1] | 187 |
| 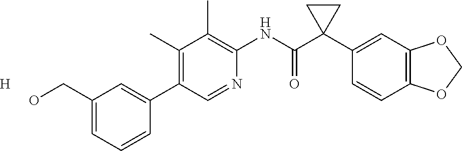 | 188 |
| 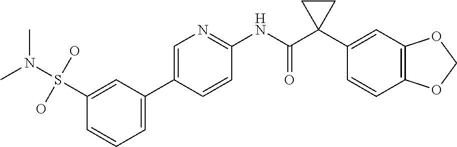 [SMILES: CN(C)S(O)(O)c1cccc(-c2ccc(NC(=O)C3(c4ccc5c(c4)OCO5)CC3)nc2)c1] | 189 |
| 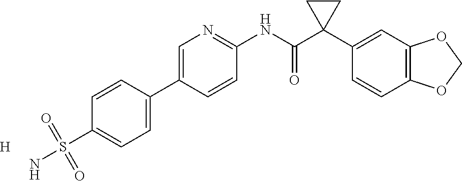 | 190 |
| 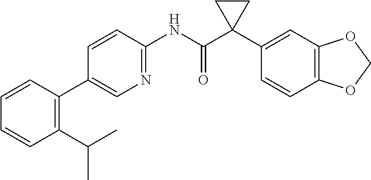 [SMILES: CC(C)c1ccccc1-c1ccc(NC(=O)C2(c3ccc4c(c3)OCO4)CC2)nc1] | 191 |
| 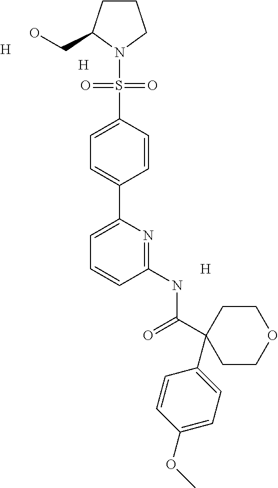 | 192 |
| 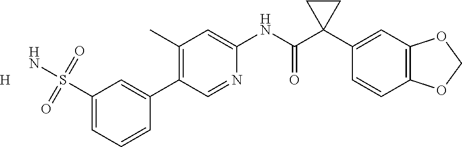 | 193 |
| 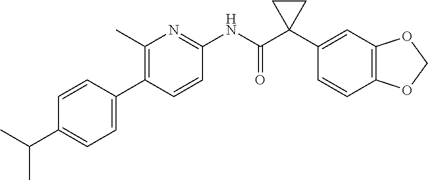 [SMILES: Cc1nc(NC(=O)C2(c3ccc4c(c3)OCO4)CC2)ccc1-c1ccc(C(C)C)cc1] | 194 |
| 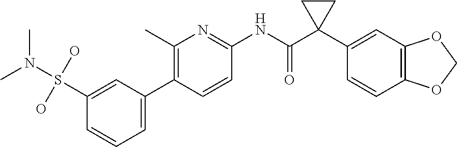 [SMILES: Cc1nc(NC(=O)C2(c3ccc4c(c3)OCO4)CC2)ccc1-c1cccc(S(O)(O)N(C)C)c1] | 195 |
| 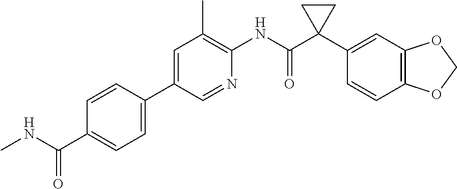 [SMILES: CNC(=O)c1ccc(-c2cnc(NC(=O)C3(c4ccc5c(c4)OCO5)CC3)c(C)c2)cc1] | 196 |
| 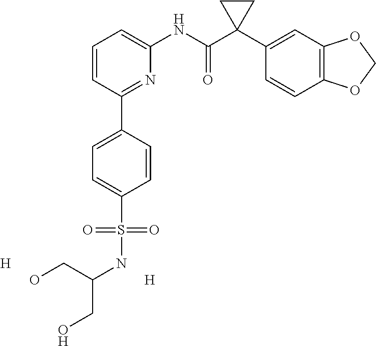 | 197 |
| 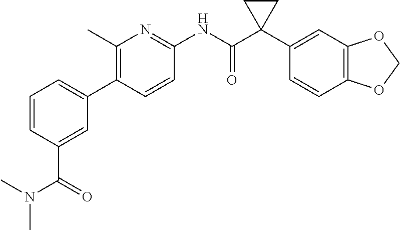 [SMILES: Cc1nc(NC(=O)C2(c3ccc4c(c3)OCO4)CC2)ccc1-c1cccc(C(=O)N(C)C)c1] | 198 |
| 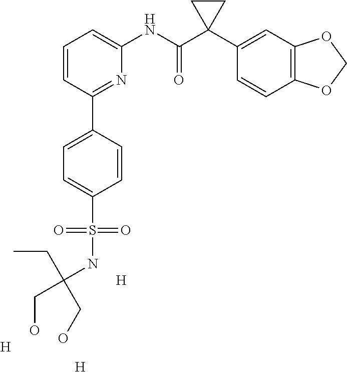 | 199 |
| 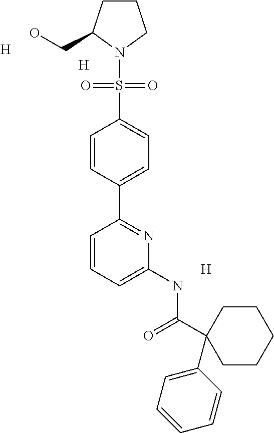 | 200 |
| 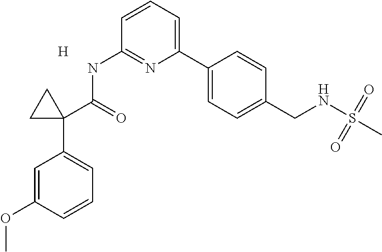 | 201 |
| 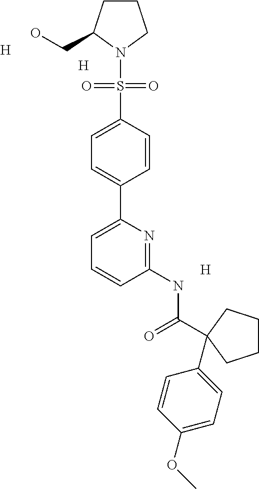 | 202 |
| 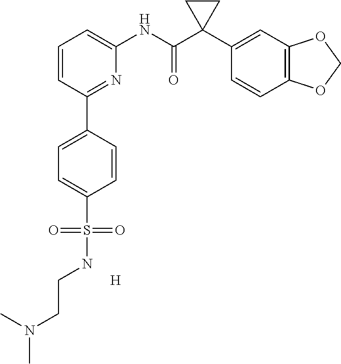 | 203 |
| 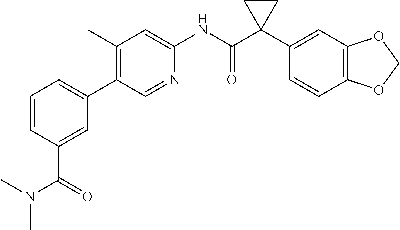 [SMILES: Cc1cc(NC(=O)C2(c3ccc4c(c3)OCO4)CC2)ncc1-c1cccc(C(=O)N(C)C)c1] | 204 |
| 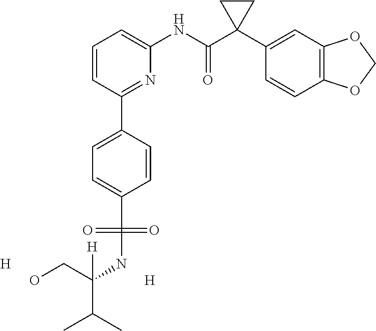 | 205 |
| 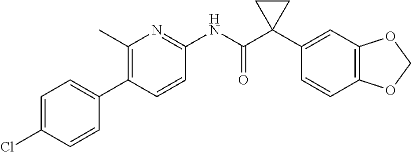 [SMILES: Cc1nc(NC(=O)C2(c3ccc4c(c3)OCO4)CC2)ccc1-c1ccc(Cl)cc1] | 206 |
| 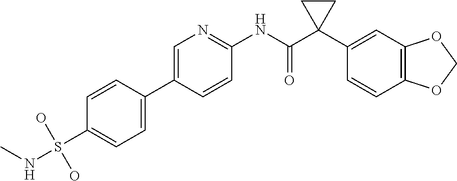 [SMILES: CNS(O)(O)c1ccc(-c2ccc(NC(=O)C3(c4ccc5c(c4)OCO5)CC3)nc2)cc1] | 207 |
| 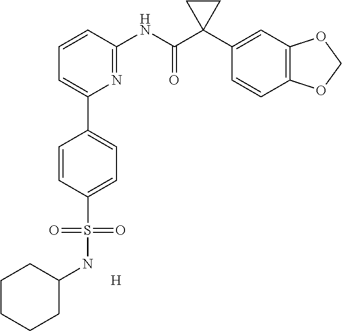 | 208 |
| 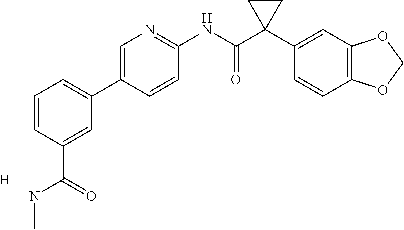 | 209 |
| 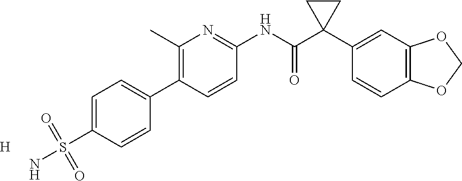 | 210 |
| 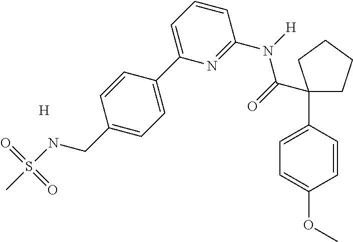 | 211 |
| 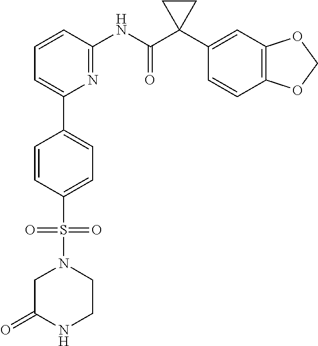 [SMILES: O=C1CN(S(=O)(=O)c2ccc(-c3cccc(NC(=O)C4(c5ccc6c(c5)OCO6)CC4)n3)cc2)CCN1] | 212 |
| 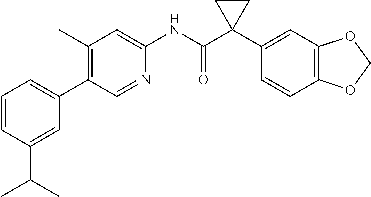 [SMILES: Cc1cc(NC(=O)C2(c3ccc4c(c3)OCO4)CC2)ncc1-c1cccc(C(C)C)c1] | 213 |
| 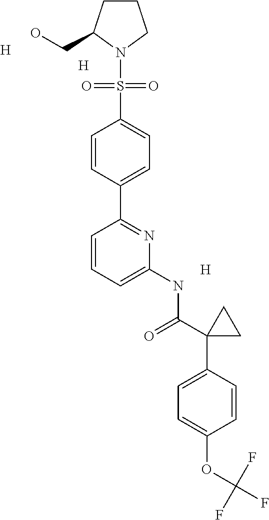 | 214 |
| 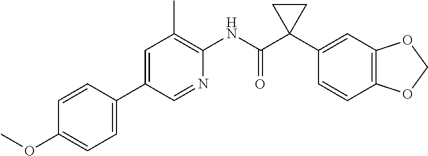 [SMILES: COc1ccc(-c2cnc(NC(=O)C3(c4ccc5c(c4)OCO5)CC3)c(C)c2)cc1] | 215 |
| 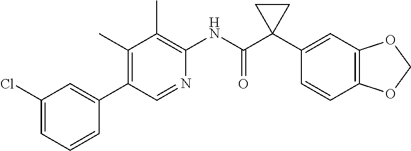 [SMILES: Cc1c(-c2cccc(Cl)c2)cnc(NC(=O)C2(c3ccc4c(c3)OCO4)CC2)c1C] | 216 |
| 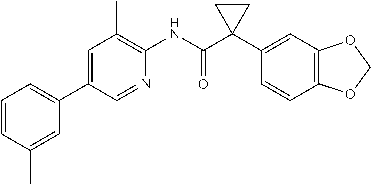 [SMILES: Cc1cccc(-c2cnc(NC(=O)C3(c4ccc5c(c4)OCO5)CC3)c(C)c2)c1] | 217 |
| 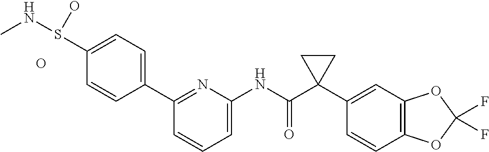 | 218 |
| 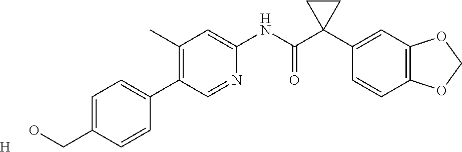 | 219 |
| 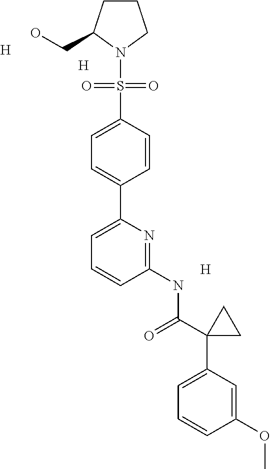 | 220 |
| 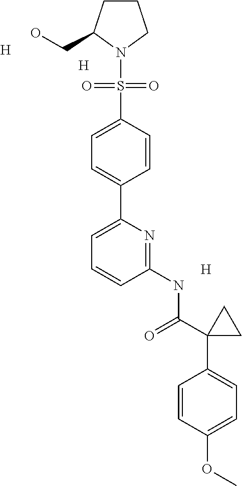 | 221 |
| 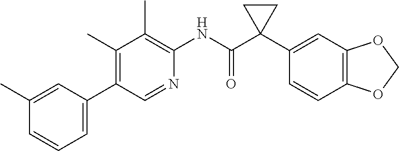 [SMILES: Cc1cccc(-c2cnc(NC(=O)C3(c4ccc5c(c4)OCO5)CC3)c(C)c2C)c1] | 222 |
| 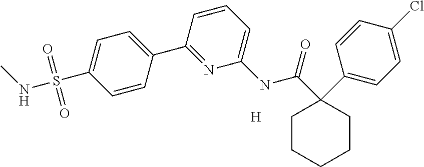 | 223 |
| 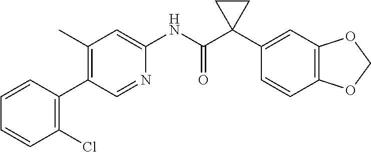 [SMILES: Cc1cc(NC(=O)C2(c3ccc4c(c3)OCO4)CC2)ncc1-c1ccccc1Cl] | 224 |
| 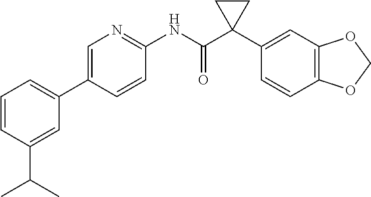 [SMILES: CC(C)c1cccc(-c2ccc(NC(=O)C3(c4ccc5c(c4)OCO5)CC3)nc2)c1] | 225 |
| 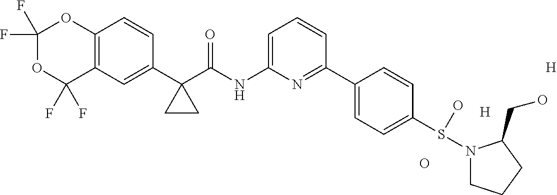 | 226 |
| 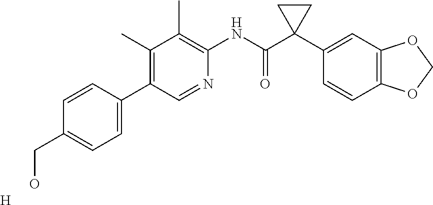 | 227 |
| 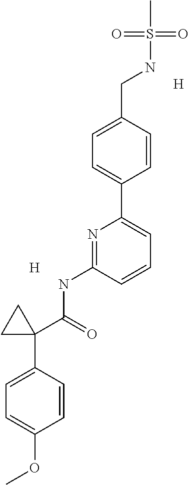 | 228 |
| 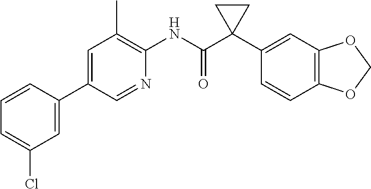 [SMILES: Cc1cc(-c2cccc(Cl)c2)cnc1NC(=O)C1(c2ccc3c(c2)OCO3)CC1] | 229 |
| 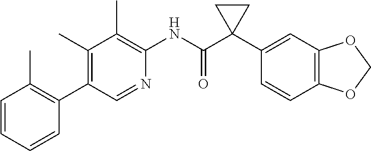 [SMILES: Cc1ccccc1-c1cnc(NC(=O)C2(c3ccc4c(c3)OCO4)CC2)c(C)c1C] | 230 |
| 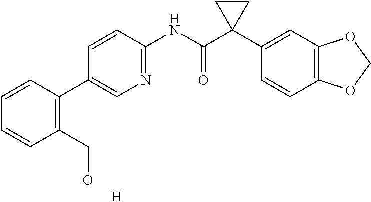 | 231 |
| 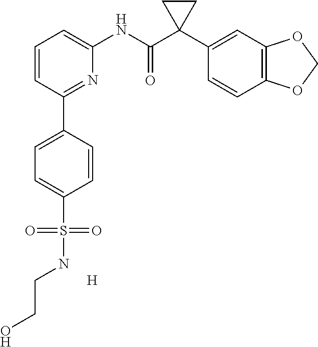 | 232 |
| 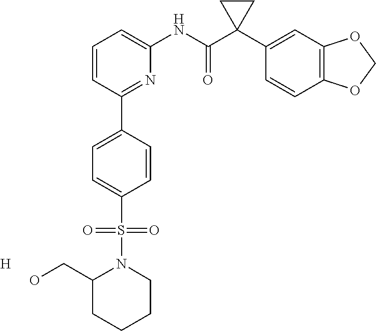 | 233 |
| 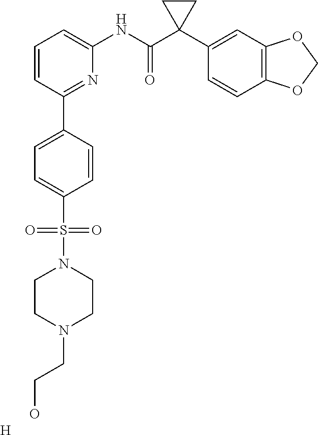 | 234 |
| 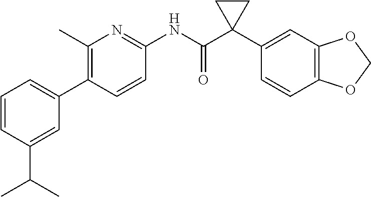 [SMILES: Cc1nc(NC(=O)C2(c3ccc4c(c3)OCO4)CC2)ccc1-c1cccc(C(C)C)c1] | 235 |
| 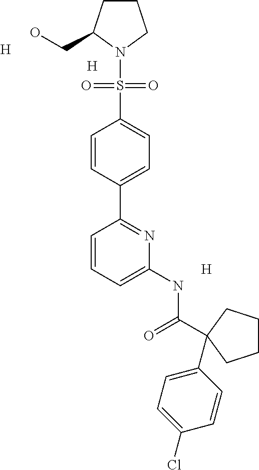 | 236 |
| 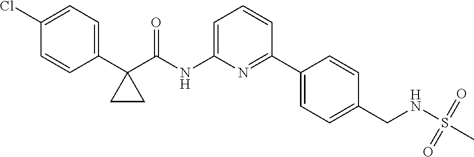 [SMILES: CS(=O)(=O)NCc1ccc(-c2cccc(NC(=O)C3(c4ccc(Cl)cc4)CC3)n2)cc1] | 237 |
| 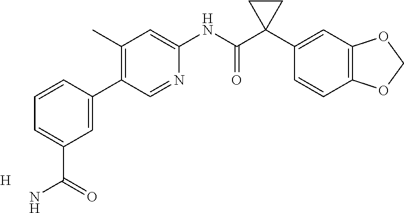 | 238 |
| 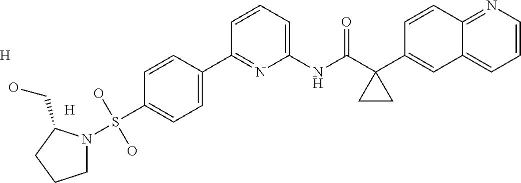 | 239 |
| 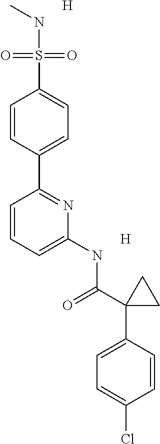 | 240 |
| 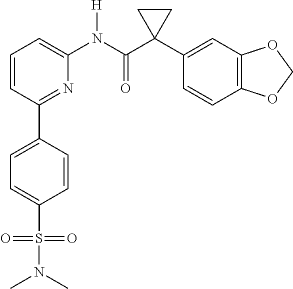 [SMILES: CN(C)S(=O)(=O)c1ccc(-c2cccc(NC(=O)C3(c4ccc5c(c4)OCO5)CC3)n2)cc1] | 241 |
| 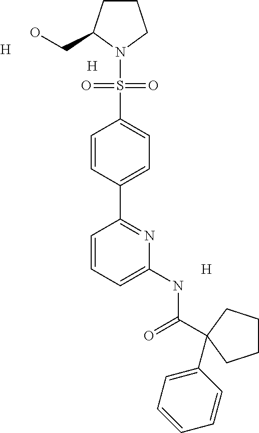 | 242 |
| 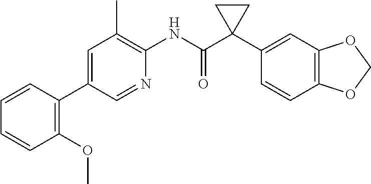 [SMILES: COc1ccccc1-c1cnc(NC(=O)C2(c3ccc4c(c3)OCO4)CC2)c(C)c1] | 243 |
| 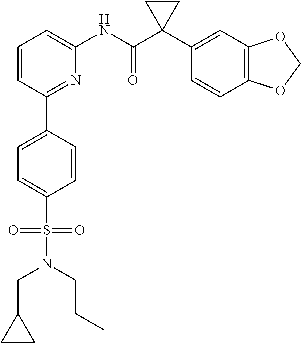 [SMILES: CCCN(CC1CC1)S(=O)(=O)c1ccc(-c2cccc(NC(=O)C3(c4ccc5c(c4)OCO5)CC3)n2)cc1] | 244 |
| 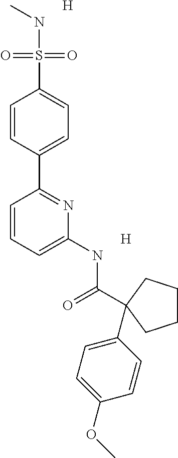 | 245 |
| 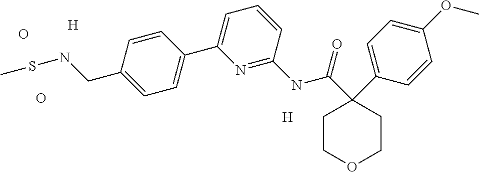 | 246 |
| 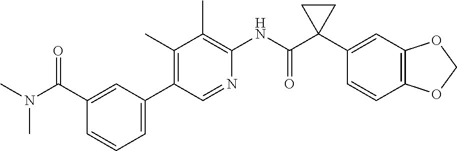 [SMILES: Cc1c(-c2cccc(C(=O)N(C)C)c2)cnc(NC(=O)C2(c3ccc4c(c3)OCO4)CC2)c1C] | 247 |
| 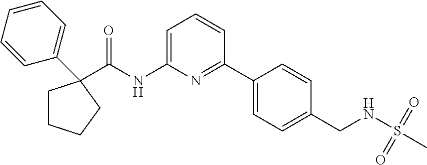 [SMILES: CS(=O)(=O)NCc1ccc(-c2cccc(NC(=O)C3(c4ccccc4)CCCC3)n2)cc1] | 248 |
| 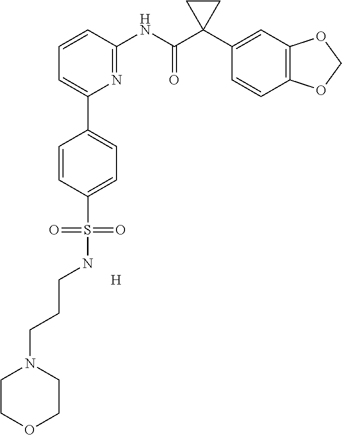 | 249 |
| 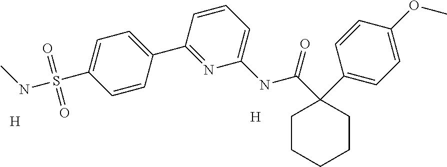 | 250 |
| 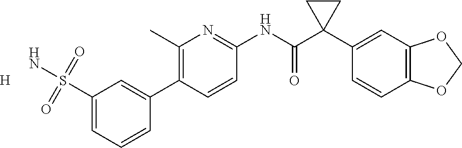 | 251 |
| 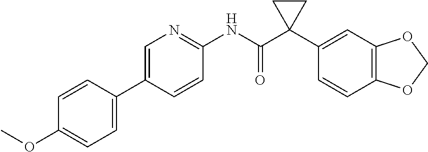 [SMILES: COc1ccc(-c2ccc(NC(=O)C3(c4ccc5c(c4)OCO5)CC3)nc2)cc1] | 252 |
| 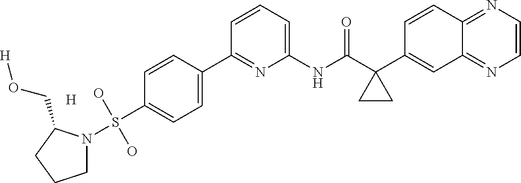 | 253 |
| 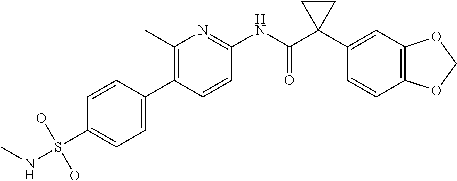 [SMILES: CNS(O)(O)c1ccc(-c2ccc(NC(=O)C3(c4ccc5c(c4)OCO5)CC3)nc2C)cc1] | 254 |
| 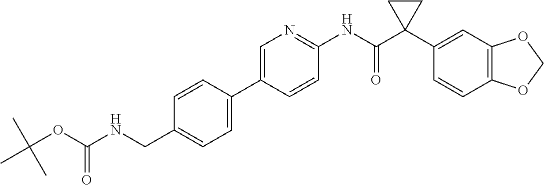 [SMILES: CC(C)(C)OC(=O)NCc1ccc(-c2ccc(NC(=O)C3(c4ccc5c(c4)OCO5)CC3)nc2)cc1] | 255 |
| 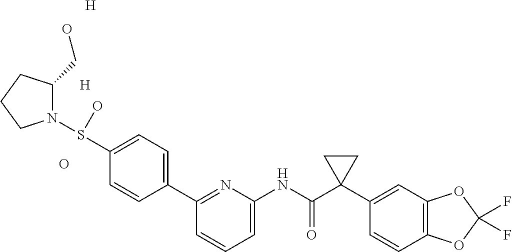 | 256 |
| 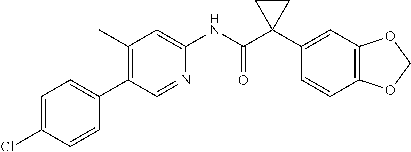 [SMILES: Cc1cc(NC(=O)C2(c3ccc4c(c3)OCO4)CC2)ncc1-c1ccc(Cl)cc1] | 257 |
| 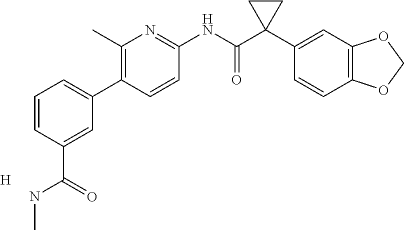 | 258 |
| 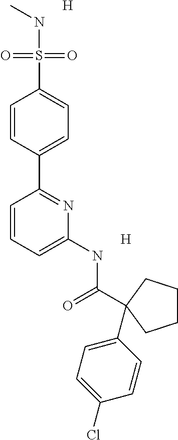 | 259 |
| 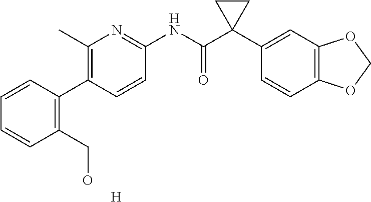 | 260 |
| 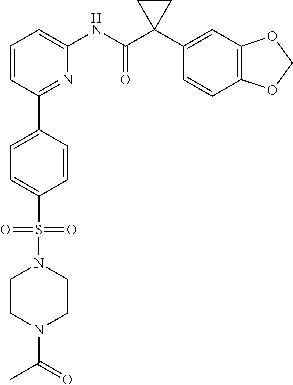 [SMILES: CC(=O)N1CCN(S(=O)(=O)c2ccc(-c3cccc(NC(=O)C4(c5ccc6c(c5)OCO6)CC4)n3)cc2)CC1] | 261 |
| 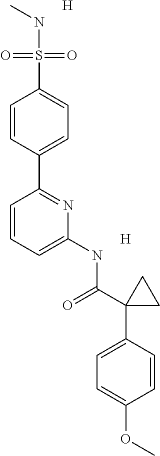 | 262 |
| 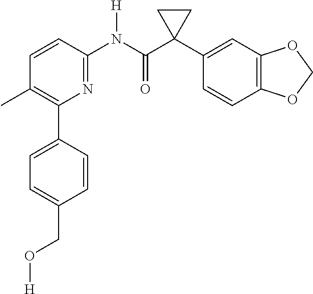 [SMILES: Cc1ccc(NC(=O)C2(c3ccc4c(c3)OCO4)CC2)nc1-c1ccc(CO)cc1] | 263 |
| 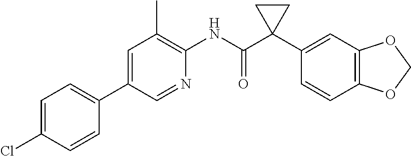 [SMILES: Cc1cc(-c2ccc(Cl)cc2)cnc1NC(=O)C1(c2ccc3c(c2)OCO3)CC1] | 264 |
| 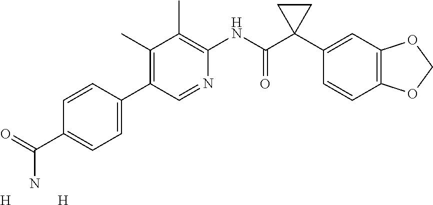 | 265 |
| 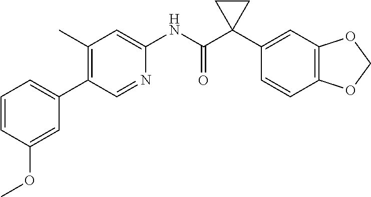 [SMILES: COc1cccc(-c2cnc(NC(=O)C3(c4ccc5c(c4)OCO5)CC3)cc2C)c1] | 266 |
| 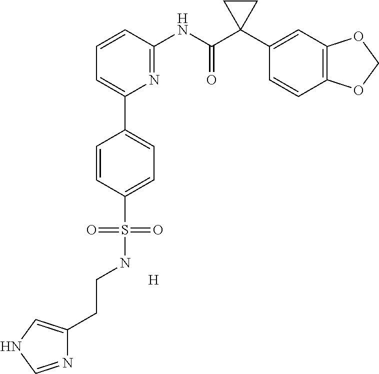 | 267 |
| 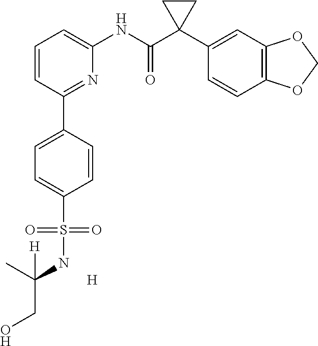 | 268 |
| 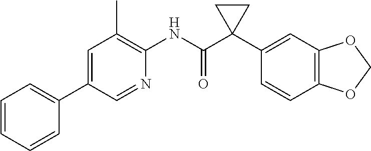 [SMILES: Cc1cc(-c2ccccc2)cnc1NC(=O)C1(c2ccc3c(c2)OCO3)CC1] | 269 |
| 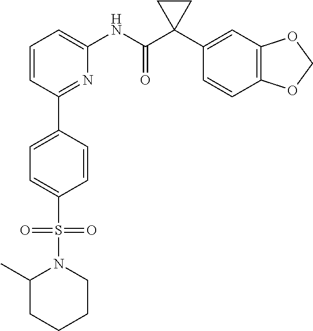 [SMILES: CC1CCCCN1S(=O)(=O)c1ccc(-c2cccc(NC(=O)C3(c4ccc5c(c4)OCO5)CC3)n2)cc1] | 270 |
| 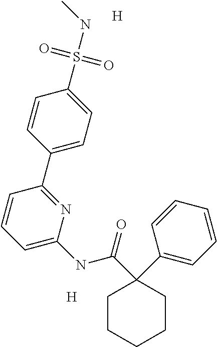 | 271 |
| 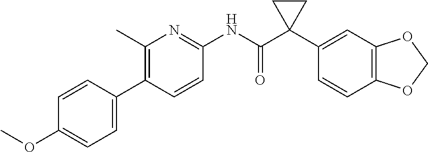 [SMILES: COc1ccc(-c2ccc(NC(=O)C3(c4ccc5c(c4)OCO5)CC3)nc2C)cc1] | 272 |
| 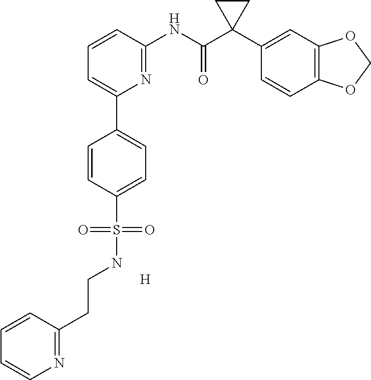 | 273 |
| 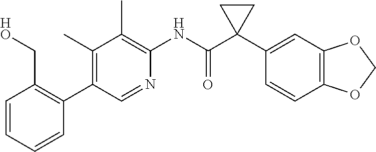 [SMILES: Cc1c(-c2ccccc2CO)cnc(NC(=O)C2(c3ccc4c(c3)OCO4)CC2)c1C] | 274 |
| 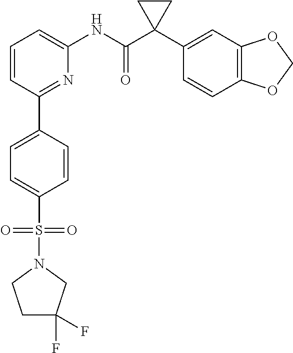 [SMILES: O=C(Nc1cccc(-c2ccc(S(=O)(=O)N3CCC(F)(F)C3)cc2)n1)C1(c2ccc3c(c2)OCO3)CC1] | 275 |
| 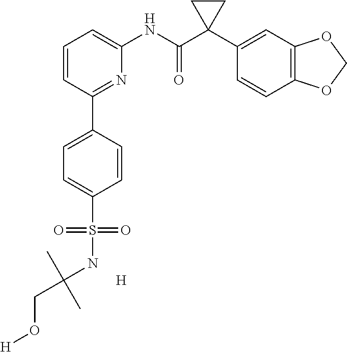 | 276 |
| 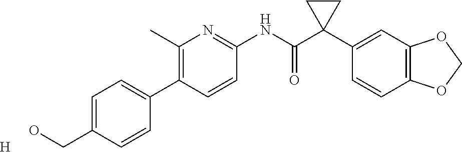 | 277 |
| 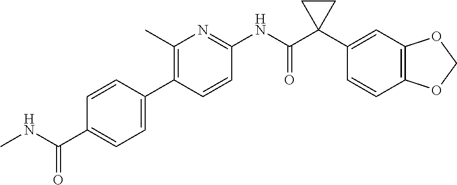 [SMILES: CNC(=O)c1ccc(-c2ccc(NC(=O)C3(c4ccc5c(c4)OCO5)CC3)nc2C)cc1] | 278 |
| 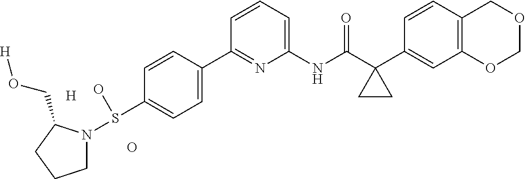 | 279 |
| 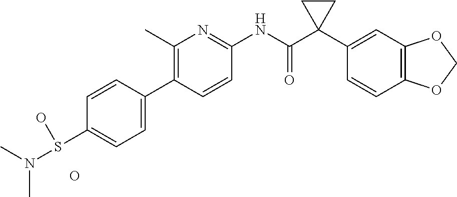 | 280 |
| 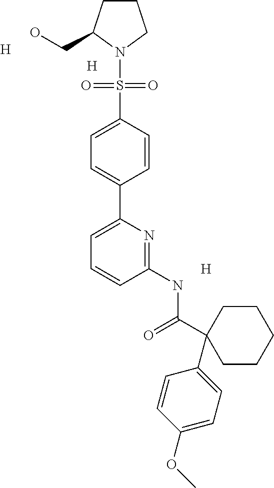 | 281 |
| 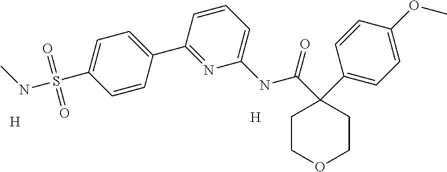 | 282 |
| 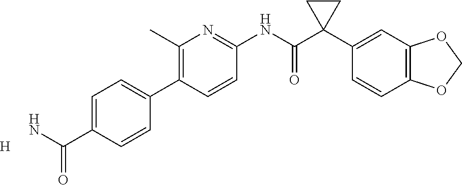 | 283 |
| 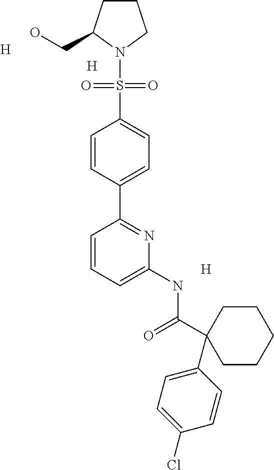 | 284 |
| 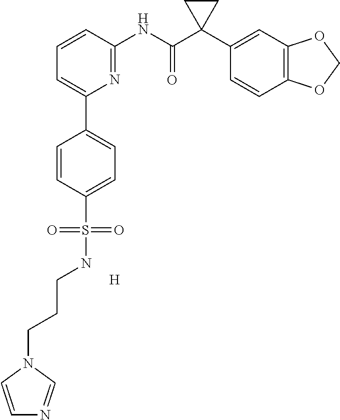 | 285 |
| 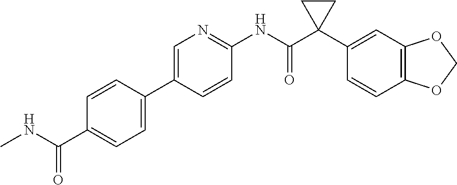 [SMILES: CNC(=O)c1ccc(-c2ccc(NC(=O)C3(c4ccc5c(c4)OCO5)CC3)nc2)cc1] | 286 |
| 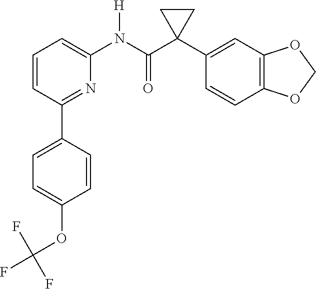 [SMILES: O=C(Nc1cccc(-c2ccc(OC(F)(F)F)cc2)n1)C1(c2ccc3c(c2)OCO3)CC1] | 287 |
| 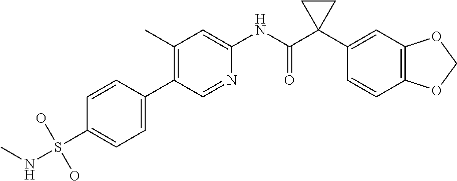 [SMILES: CNS(O)(O)c1ccc(-c2cnc(NC(=O)C3(c4ccc5c(c4)OCO5)CC3)cc2C)cc1] | 288 |
| 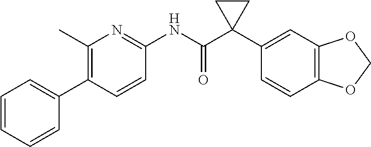 [SMILES: Cc1nc(NC(=O)C2(c3ccc4c(c3)OCO4)CC2)ccc1-c1ccccc1] | 289 |
| 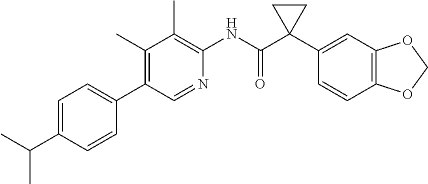 [SMILES: Cc1c(-c2ccc(C(C)C)cc2)cnc(NC(=O)C2(c3ccc4c(c3)OCO4)CC2)c1C] | 290 |
| 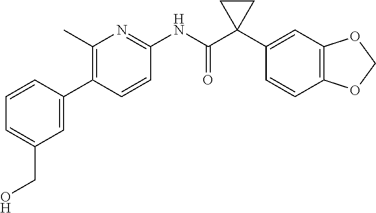 [SMILES: Cc1nc(NC(=O)C2(c3ccc4c(c3)OCO4)CC2)ccc1-c1cccc(CO)c1] | 291 |
| 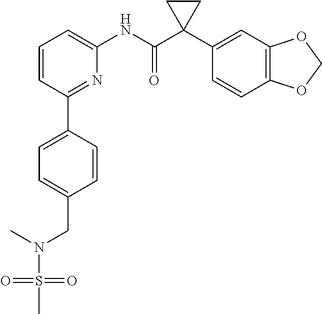 [SMILES: CN(Cc1ccc(-c2cccc(NC(=O)C3(c4ccc5c(c4)OCO5)CC3)n2)cc1)S(C)(=O)=O] | 292 |
| 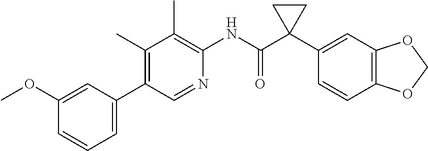 [SMILES: COc1cccc(-c2cnc(NC(=O)C3(c4ccc5c(c4)OCO5)CC3)c(C)c2C)c1] | 293 |
| 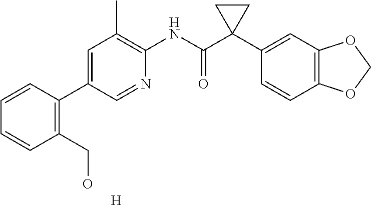 | 294 |
| 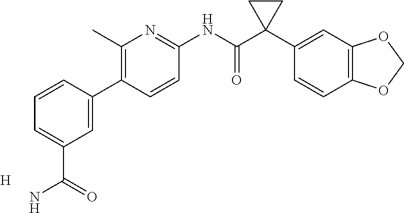 | 295 |
| 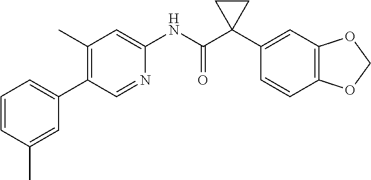 [SMILES: Cc1cccc(-c2cnc(NC(=O)C3(c4ccc5c(c4)OCO5)CC3)cc2C)c1] | 296 |
| 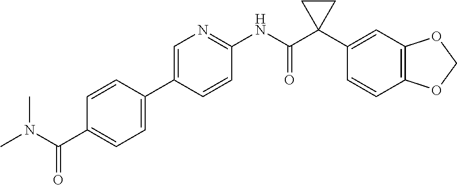 [SMILES: CN(C)C(=O)c1ccc(-c2ccc(NC(=O)C3(c4ccc5c(c4)OCO5)CC3)nc2)cc1] | 297 |
| 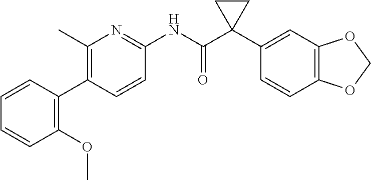 [SMILES: COc1ccccc1-c1ccc(NC(=O)C2(c3ccc4c(c3)OCO4)CC2)nc1C] | 298 |
| 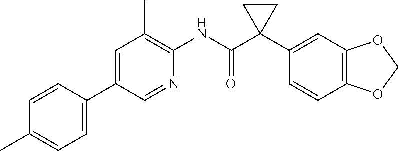 [SMILES: Cc1ccc(-c2cnc(NC(=O)C3(c4ccc5c(c4)OCO5)CC3)c(C)c2)cc1] | 299 |
| 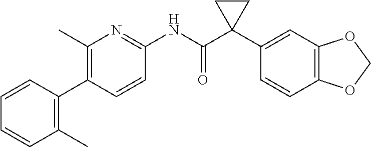 [SMILES: Cc1ccccc1-c1ccc(NC(=O)C2(c3ccc4c(c3)OCO4)CC2)nc1C] | 300 |
| 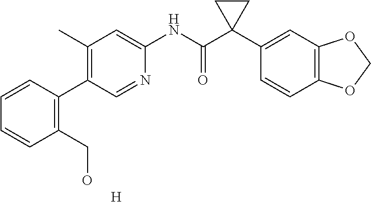 | 301 |
| 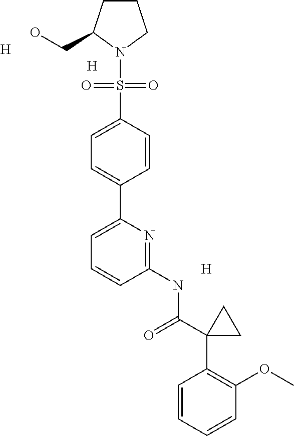 | 302 |
| 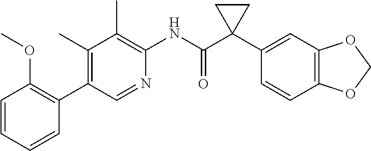 [SMILES: COc1ccccc1-c1cnc(NC(=O)C2(c3ccc4c(c3)OCO4)CC2)c(C)c1C] | 303 |
| 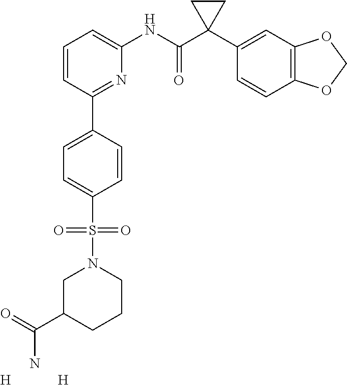 | 304 |
| 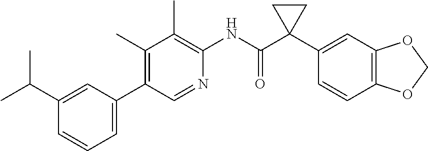 [SMILES: Cc1c(-c2cccc(C(C)C)c2)cnc(NC(=O)C2(c3ccc4c(c3)OCO4)CC2)c1C] | 305 |
| 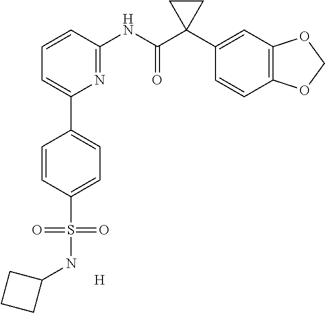 | 306 |
| 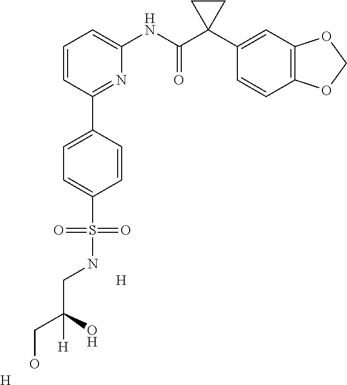 | 307 |
| 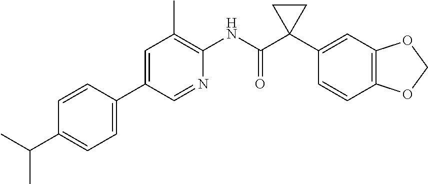 [SMILES: Cc1cc(-c2ccc(C(C)C)cc2)cnc1NC(=O)C1(c2ccc3c(c2)OCO3)CC1] | 308 |
| 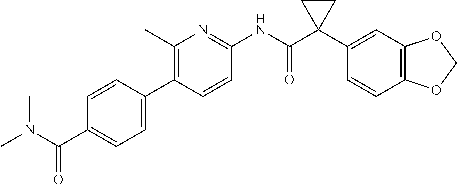 [SMILES: Cc1nc(NC(=O)C2(c3ccc4c(c3)OCO4)CC2)ccc1-c1ccc(C(=O)N(C)C)cc1] | 309 |
| 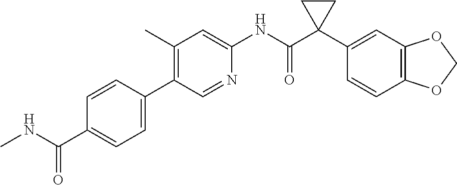 [SMILES: CNC(=O)c1ccc(-c2cnc(NC(=O)C3(c4ccc5c(c4)OCO5)CC3)cc2C)cc1] | 310 |
| 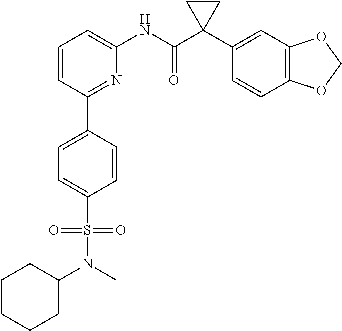 [SMILES: CN(C1CCCCC1)S(=O)(=O)c1ccc(-c2cccc(NC(=O)C3(c4ccc5c(c4)OCO5)CC3)n2)cc1] | 311 |
| 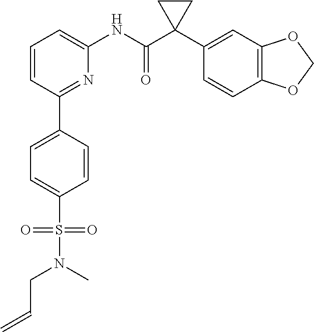 [SMILES: C=CCN(C)S(=O)(=O)c1ccc(-c2cccc(NC(=O)C3(c4ccc5c(c4)OCO5)CC3)n2)cc1] | 312 |
| 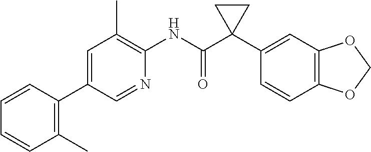 [SMILES: Cc1ccccc1-c1cnc(NC(=O)C2(c3ccc4c(c3)OCO4)CC2)c(C)c1] | 313 |
| 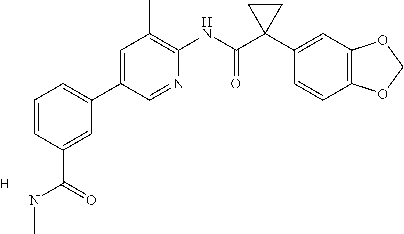 | 314 |
| 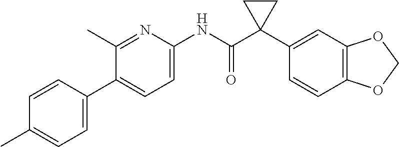 [SMILES: Cc1ccc(-c2ccc(NC(=O)C3(c4ccc5c(c4)OCO5)CC3)nc2C)cc1] | 315 |
| 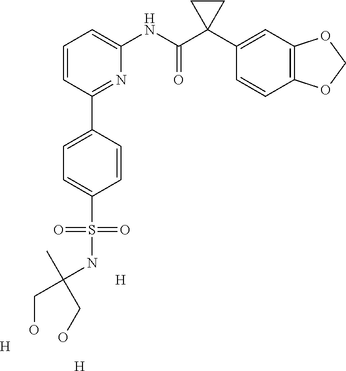 | 316 |
| 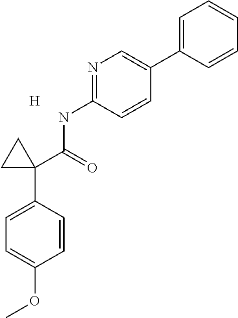 | 317 |
| 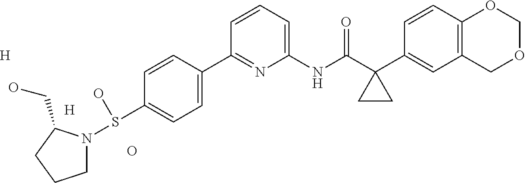 | 318 |
| 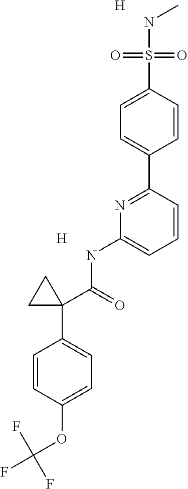 | 319 |
| 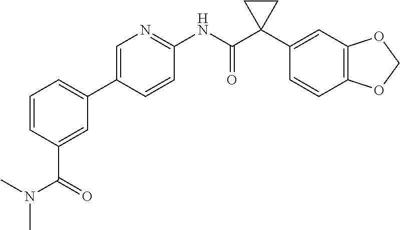 [SMILES: CN(C)C(=O)c1cccc(-c2ccc(NC(=O)C3(c4ccc5c(c4)OCO5)CC3)nc2)c1] | 320 |
| 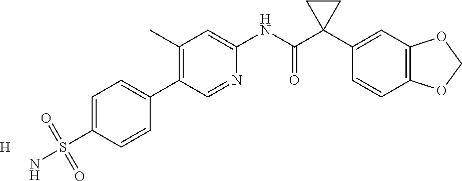 | 321 |
| 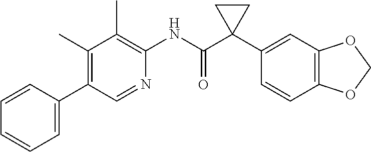 [SMILES: Cc1c(-c2ccccc2)cnc(NC(=O)C2(c3ccc4c(c3)OCO4)CC2)c1C] | 322 |
| 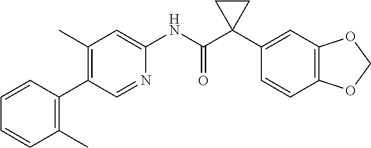 [SMILES: Cc1ccccc1-c1cnc(NC(=O)C2(c3ccc4c(c3)OCO4)CC2)cc1C] | 323 |
| 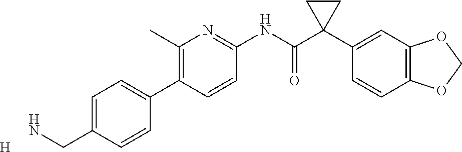 | 324 |
| 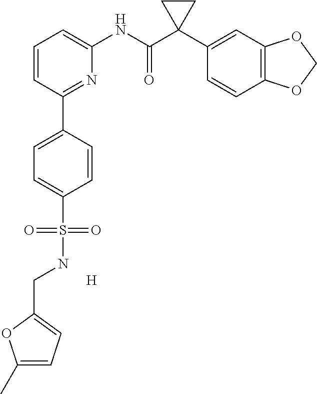 | 325 |
| 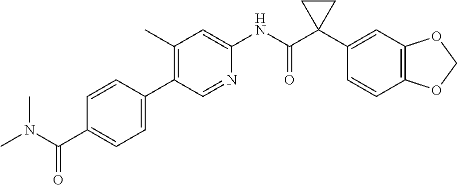 [SMILES: Cc1cc(NC(=O)C2(c3ccc4c(c3)OCO4)CC2)ncc1-c1ccc(C(=O)N(C)C)cc1] | 326 |
| 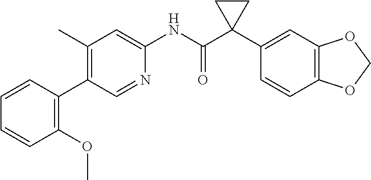 [SMILES: COc1ccccc1-c1cnc(NC(=O)C2(c3ccc4c(c3)OCO4)CC2)cc1C] | 327 |
| 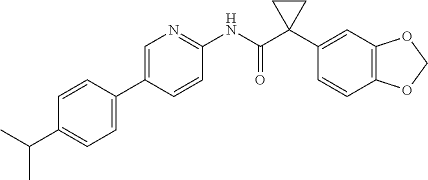 [SMILES: CC(C)c1ccc(-c2ccc(NC(=O)C3(c4ccc5c(c4)OCO5)CC3)nc2)cc1] | 328 |
| 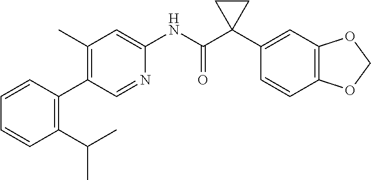 [SMILES: Cc1cc(NC(=O)C2(c3ccc4c(c3)OCO4)CC2)ncc1-c1ccccc1C(C)C] | 329 |
| 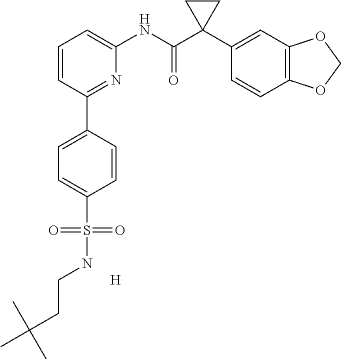 | 330 |
| 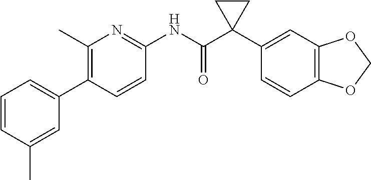 [SMILES: Cc1cccc(-c2ccc(NC(=O)C3(c4ccc5c(c4)OCO5)CC3)nc2C)c1] | 331 |
| 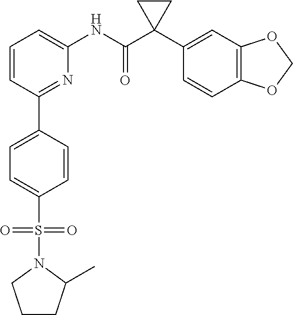 [SMILES: CC1CCCN1S(=O)(=O)c1ccc(-c2cccc(NC(=O)C3(c4ccc5c(c4)OCO5)CC3)n2)cc1] | 332 |
| 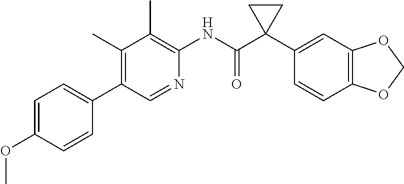 [SMILES: COc1ccc(-c2cnc(NC(=O)C3(c4ccc5c(c4)OCO5)CC3)c(C)c2C)cc1] | 333 |
| 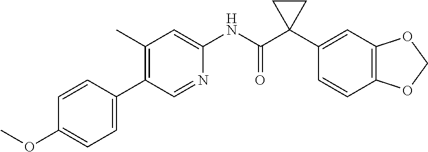 [SMILES: COc1ccc(-c2cnc(NC(=O)C3(c4ccc5c(c4)OCO5)CC3)cc2C)cc1] | 334 |
| 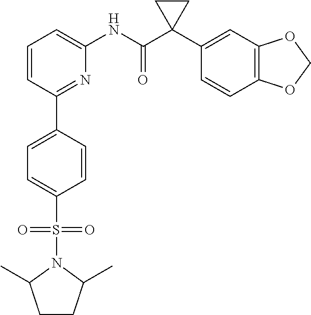 [SMILES: CC1CCC(C)N1S(=O)(=O)c1ccc(-c2cccc(NC(=O)C3(c4ccc5c(c4)OCO5)CC3)n2)cc1] | 335 |
| 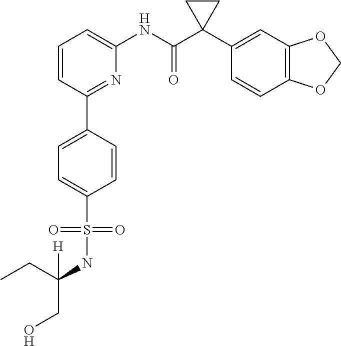 [SMILES: CC[C@H](CO)NS(=O)(=O)c1ccc(-c2cccc(NC(=O)C3(c4ccc5c(c4)OCO5)CC3)n2)cc1] | 336 |
| 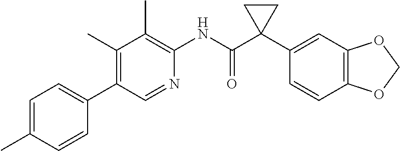 [SMILES: Cc1ccc(-c2cnc(NC(=O)C3(c4ccc5c(c4)OCO5)CC3)c(C)c2C)cc1] | 337 |
| 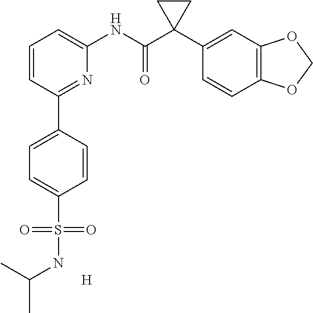 | 338 |
| 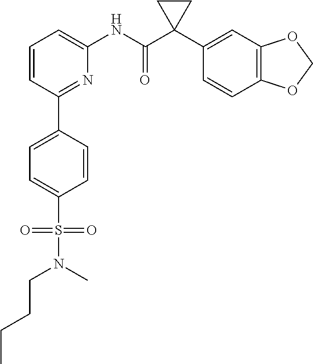 [SMILES: CCCCN(C)S(=O)(=O)c1ccc(-c2cccc(NC(=O)C3(c4ccc5c(c4)OCO5)CC3)n2)cc1] | 339 |
| 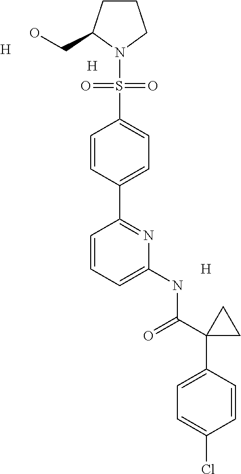 | 340 |
| 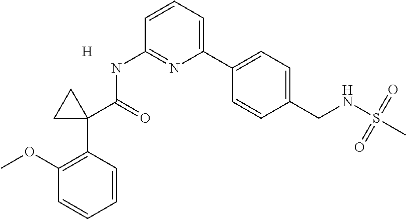 | 341 |
| 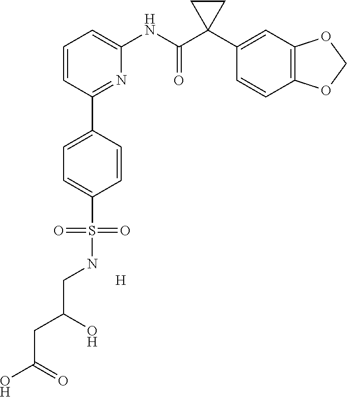 | 342 |
| 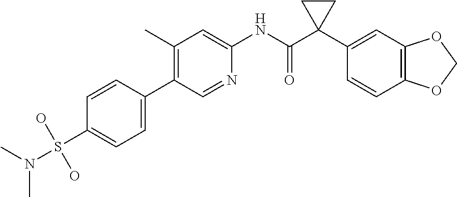 [SMILES: Cc1cc(NC(=O)C2(c3ccc4c(c3)OCO4)CC2)ncc1-c1ccc(S(O)(O)N(C)C)cc1] | 343 |
| 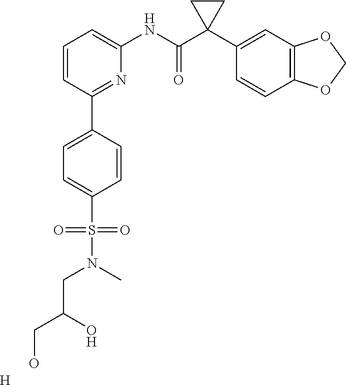 | 344 |
| 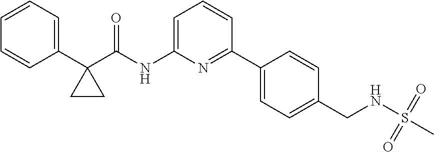 [SMILES: CS(=O)(=O)NCc1ccc(-c2cccc(NC(=O)C3(c4ccccc4)CC3)n2)cc1] | 345 |
| 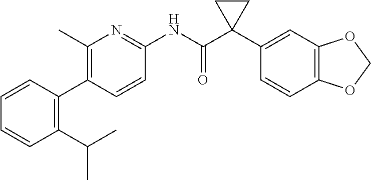 [SMILES: Cc1nc(NC(=O)C2(c3ccc4c(c3)OCO4)CC2)ccc1-c1ccccc1C(C)C] | 346 |
| 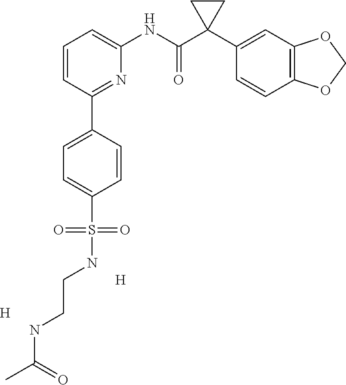 | 347 |
| 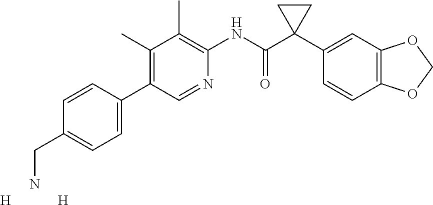 | 348 |
| 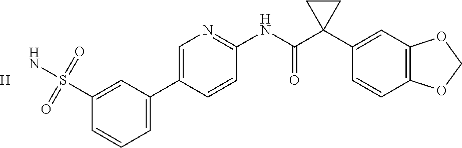 | 349 |
| 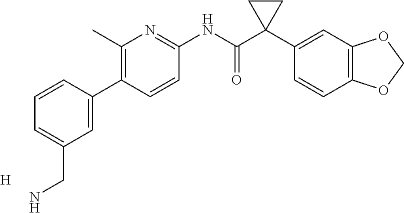 | 350 |
| 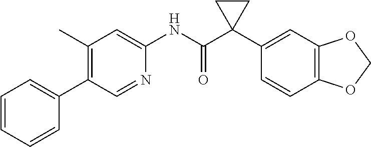 [SMILES: Cc1cc(NC(=O)C2(c3ccc4c(c3)OCO4)CC2)ncc1-c1ccccc1] | 351 |
| 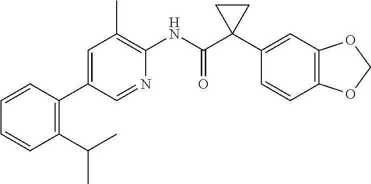 [SMILES: Cc1cc(-c2ccccc2C(C)C)cnc1NC(=O)C1(c2ccc3c(c2)OCO3)CC1] | 352 |
| 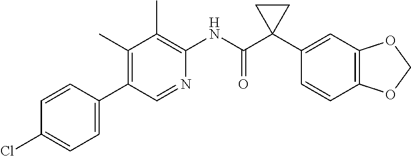 [SMILES: Cc1c(-c2ccc(Cl)cc2)cnc(NC(=O)C2(c3ccc4c(c3)OCO4)CC2)c1C] | 353 |
| 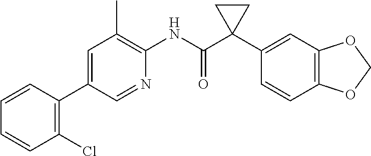 [SMILES: Cc1cc(-c2ccccc2Cl)cnc1NC(=O)C1(c2ccc3c(c2)OCO3)CC1] | 354 |
| 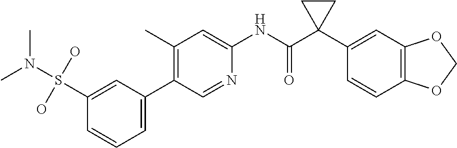 [SMILES: Cc1cc(NC(=O)C2(c3ccc4c(c3)OCO4)CC2)ncc1-c1cccc(S(O)(O)N(C)C)c1] | 355 |
| 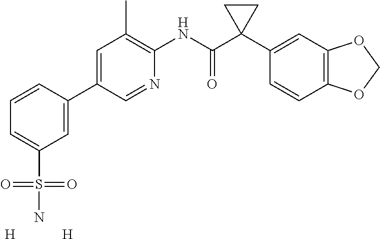 | 356 |
| 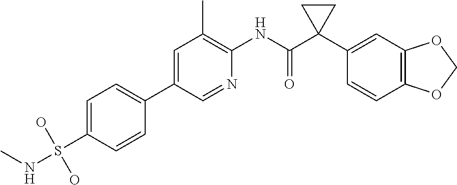 [SMILES: CNS(O)(O)c1ccc(-c2cnc(NC(=O)C3(c4ccc5c(c4)OCO5)CC3)c(C)c2)cc1] | 357 |
| 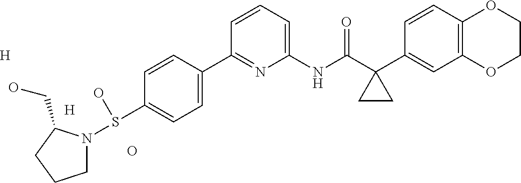 | 358 |
| 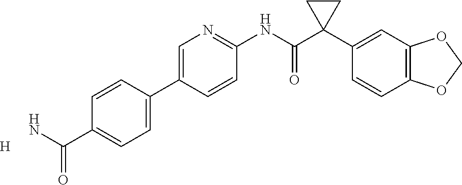 | 359 |
| 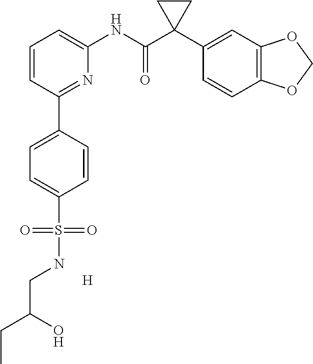 | 360 |
| 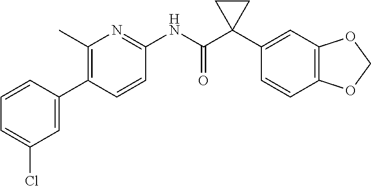 [SMILES: Cc1nc(NC(=O)C2(c3ccc4c(c3)OCO4)CC2)ccc1-c1cccc(Cl)c1] | 361 |
| 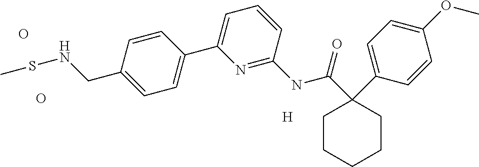 | 362 |
| 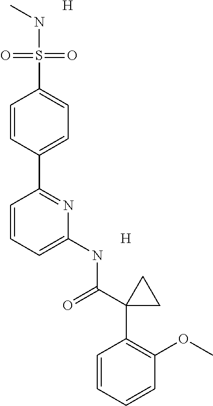 | 363 |
| 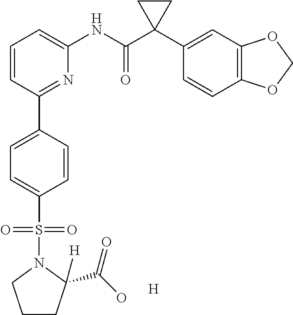 | 364 |
| 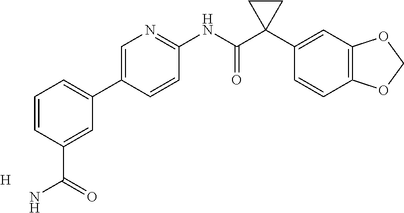 | 365 |
| 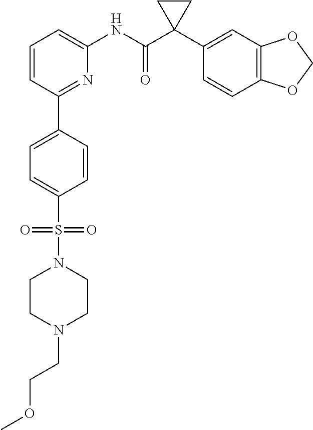 [SMILES: COCCN1CCN(S(=O)(=O)c2ccc(-c3cccc(NC(=O)C4(c5ccc6c(c5)OCO6)CC4)n3)cc2)CC1] | 366 |
| 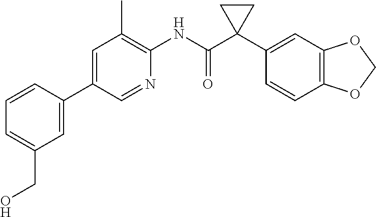 [SMILES: Cc1cc(-c2cccc(CO)c2)cnc1NC(=O)C1(c2ccc3c(c2)OCO3)CC1] | 367 |
| 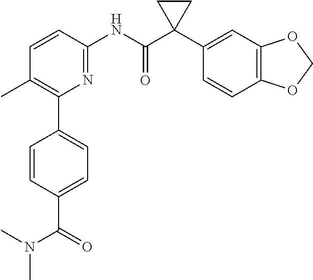 [SMILES: Cc1ccc(NC(=O)C2(c3ccc4c(c3)OCO4)CC2)nc1-c1ccc(C(=O)N(C)C)cc1] | 368 |
| 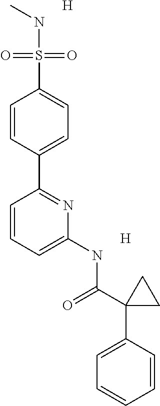 | 369 |
| 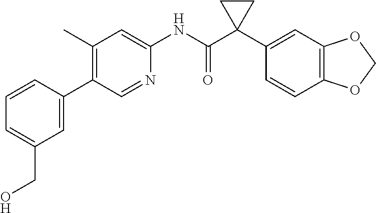 [SMILES: Cc1cc(NC(=O)C2(c3ccc4c(c3)OCO4)CC2)ncc1-c1cccc(CO)c1] | 370 |
| 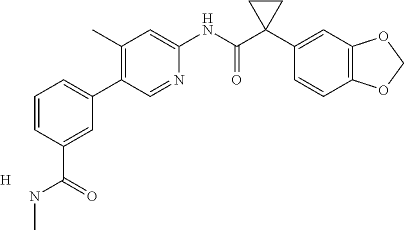 | 371 |
| 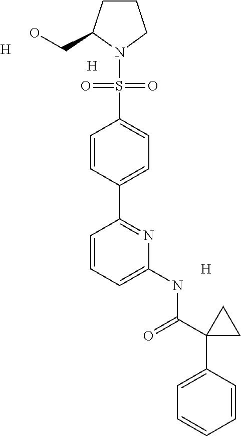 | 372 |
| 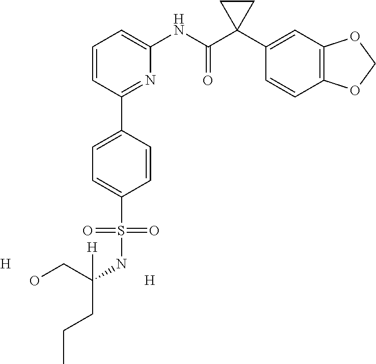 | 373 |
| 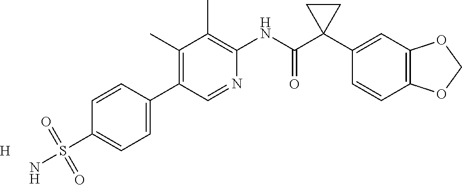 | 374 |
| 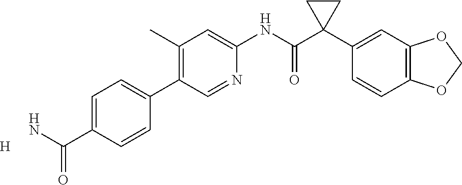 | 375 |
| 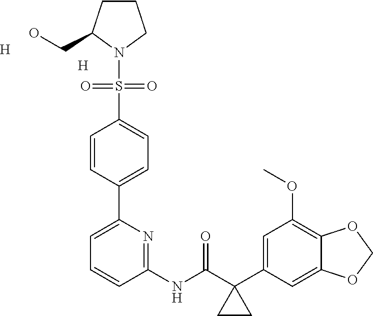 | 376 |
| 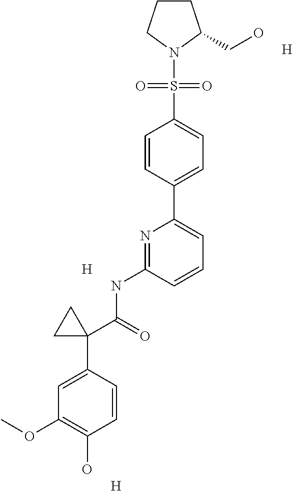 | 377 |
| 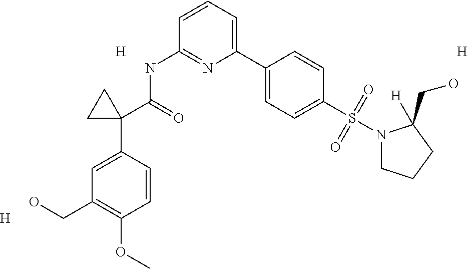 | 378 |
| 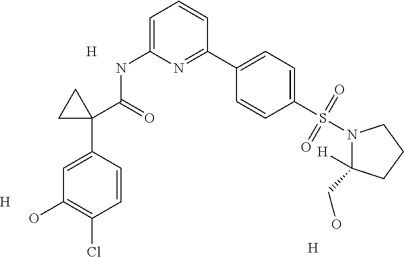 | 379 |
| 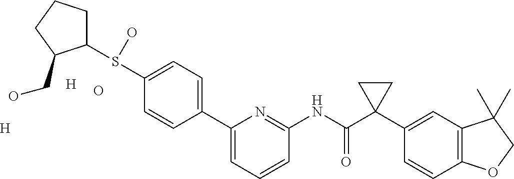 | 380 |
| 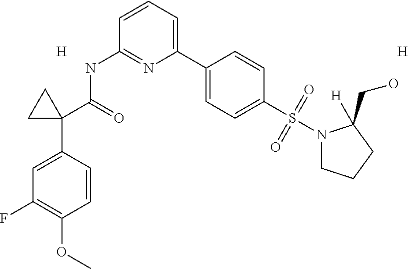 | 381 |
| 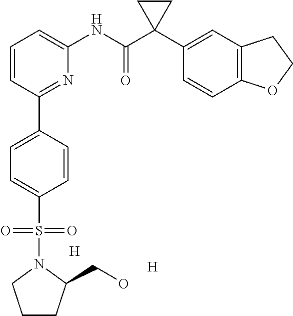 | 382 |
| 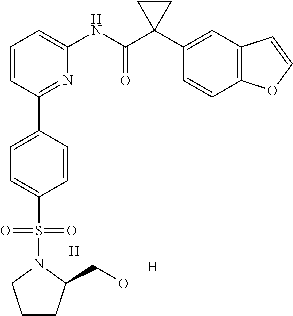 | 383 |
| 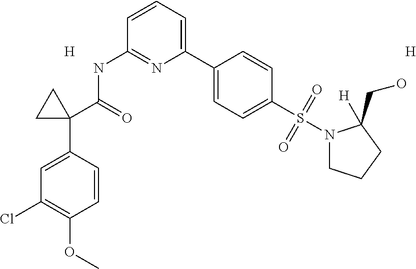 | 384 |
| 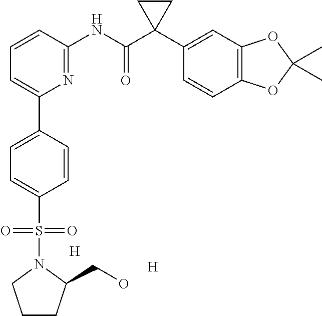 | 385 |
| 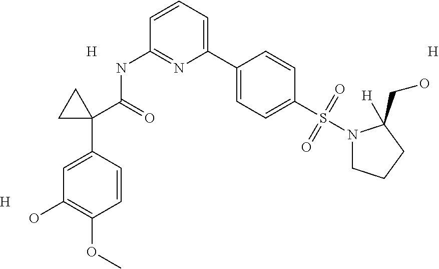 | 386 |
| 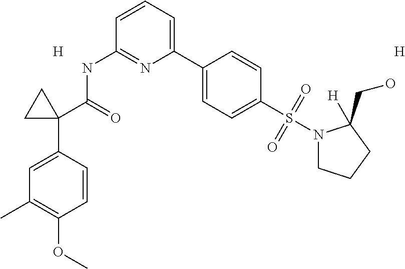 | 387 |
| 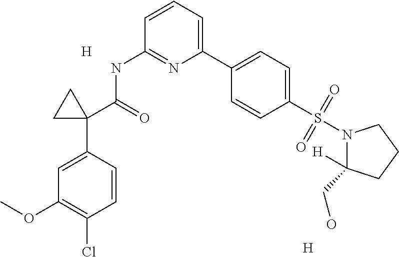 | 388 |
| 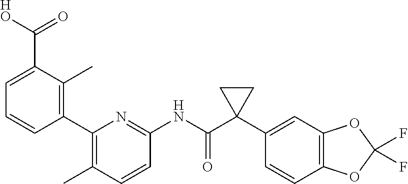 [SMILES: Cc1ccc(NC(=O)C2(c3ccc4c(c3)OC(F)(F)O4)CC2)nc1-c1cccc(C(=O)O)c1C] | 389 |
| 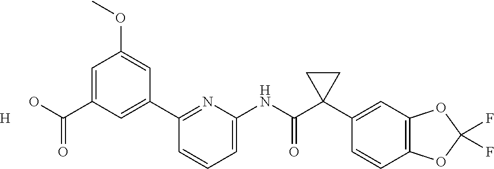 | 390 |
| 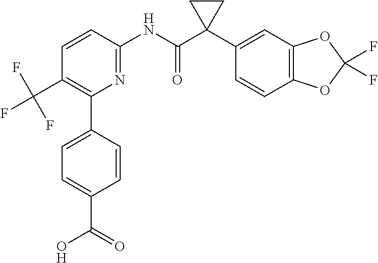 [SMILES: O=C(O)c1ccc(-c2nc(NC(=O)C3(c4ccc5c(c4)OC(F)(F)O5)CC3)ccc2C(F)(F)F)cc1] | 391 |
| 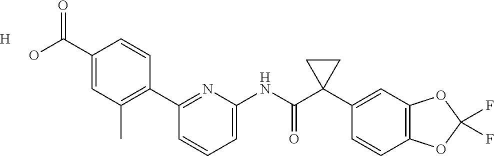 | 392 |
| 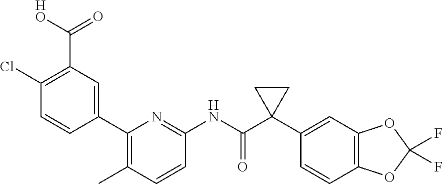 [SMILES: Cc1ccc(NC(=O)C2(c3ccc4c(c3)OC(F)(F)O4)CC2)nc1-c1ccc(Cl)c(C(=O)O)c1] | 393 |
| 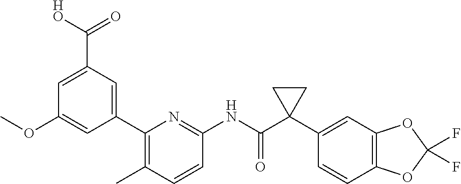 [SMILES: COc1cc(C(=O)O)cc(-c2nc(NC(=O)C3(c4ccc5c(c4)OC(F)(F)O5)CC3)ccc2C)c1] | 394 |
| 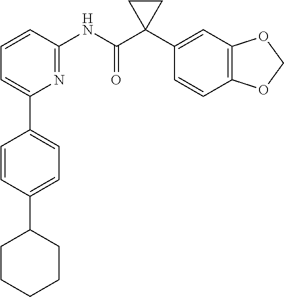 [SMILES: O=C(Nc1cccc(-c2ccc(C3CCCCC3)cc2)n1)C1(c2ccc3c(c2)OCO3)CC1] | 395 |
| 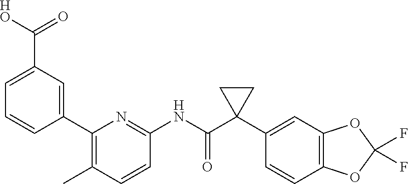 [SMILES: Cc1ccc(NC(=O)C2(c3ccc4c(c3)OC(F)(F)O4)CC2)nc1-c1cccc(C(=O)O)c1] | 396 |
| 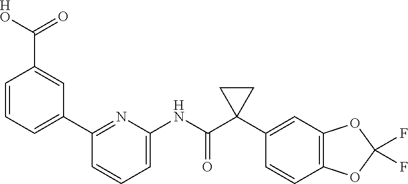 [SMILES: O=C(O)c1cccc(-c2cccc(NC(=O)C3(c4ccc5c(c4)OC(F)(F)O5)CC3)n2)c1] | 397 |
| 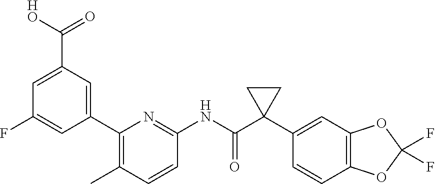 [SMILES: Cc1ccc(NC(=O)C2(c3ccc4c(c3)OC(F)(F)O4)CC2)nc1-c1cc(F)cc(C(=O)O)c1] | 398 |
| 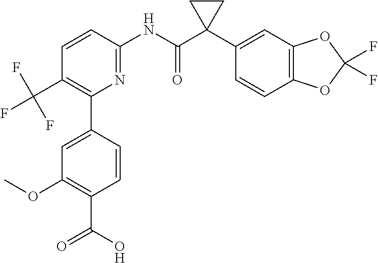 [SMILES: COc1cc(-c2nc(NC(=O)C3(c4ccc5c(c4)OC(F)(F)O5)CC3)ccc2C(F)(F)F)ccc1C(=O)O] | 399 |
| 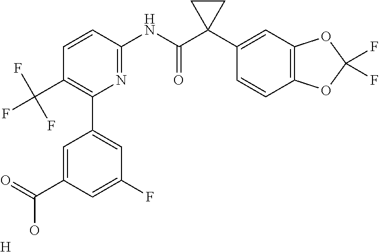 | 400 |
| 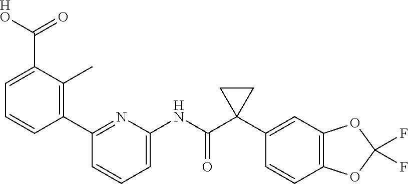 [SMILES: Cc1c(C(=O)O)cccc1-c1cccc(NC(=O)C2(c3ccc4c(c3)OC(F)(F)O4)CC2)n1] | 401 |
| 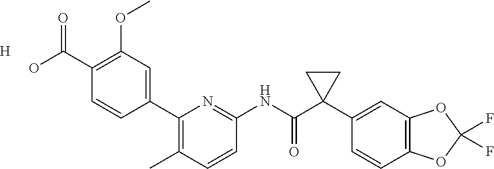 | 402 |
| 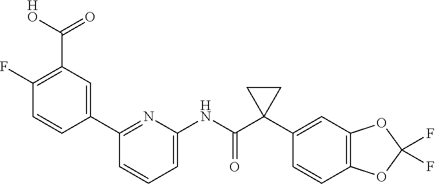 [SMILES: O=C(O)c1cc(-c2cccc(NC(=O)C3(c4ccc5c(c4)OC(F)(F)O5)CC3)n2)ccc1F] | 403 |
| 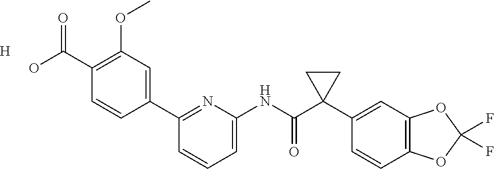 | 404 |
| 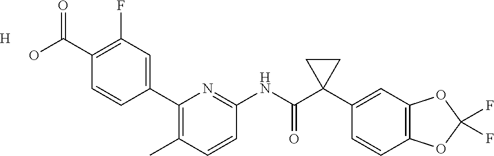 | 405 |
| 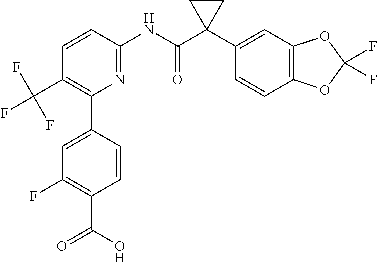 [SMILES: O=C(O)c1ccc(-c2nc(NC(=O)C3(c4ccc5c(c4)OC(F)(F)O5)CC3)ccc2C(F)(F)F)cc1F] | 406 |
| 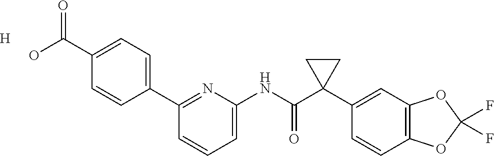 | 407 |
| 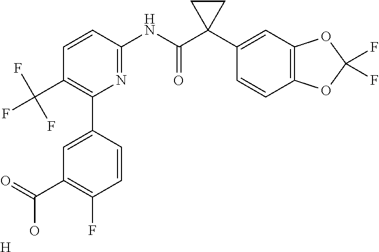 | 408 |
| 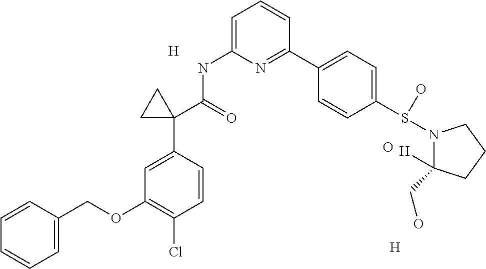 | 409 |
| 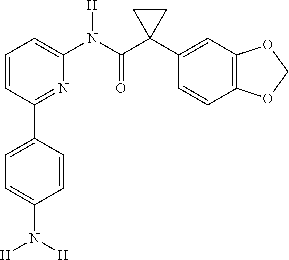 [SMILES: Nc1ccc(-c2cccc(NC(=O)C3(c4ccc5c(c4)OCO5)CC3)n2)cc1] | 410 |
| 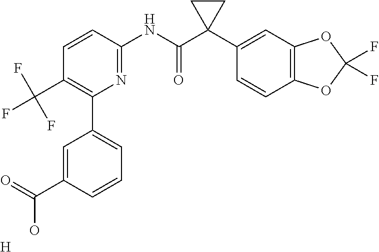 | 411 |
| 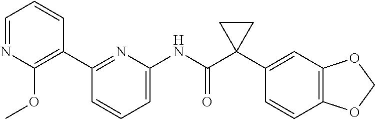 [SMILES: COc1ncccc1-c1cccc(NC(=O)C2(c3ccc4c(c3)OCO4)CC2)n1] | 412 |
| 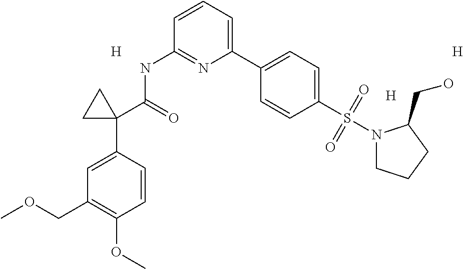 | 413 |
| 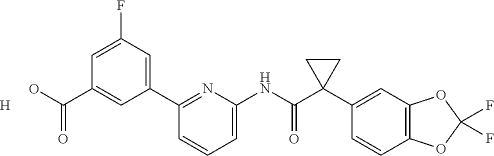 | 414 |
| 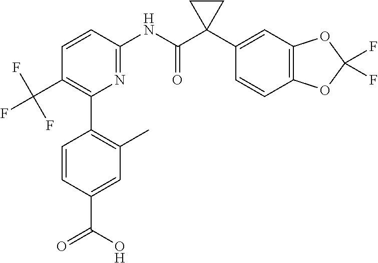 [SMILES: Cc1cc(C(=O)O)ccc1-c1nc(NC(=O)C2(c3ccc4c(c3)OC(F)(F)O4)CC2)ccc1C(F)(F)F] | 415 |
| 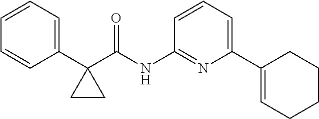 [SMILES: O=C(Nc1cccc(C2=CCCCC2)n1)C1(c2ccccc2)CC1] | 416 |
| 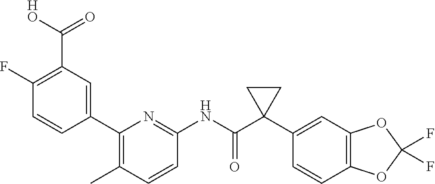 [SMILES: Cc1ccc(NC(=O)C2(c3ccc4c(c3)OC(F)(F)O4)CC2)nc1-c1ccc(F)c(C(=O)O)c1] | 417 |
| 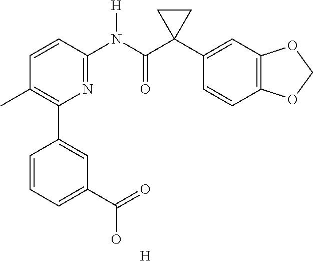 | 418 |
| 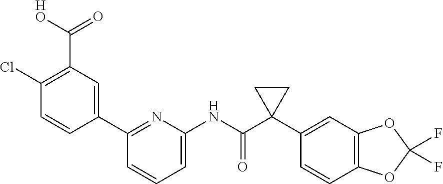 [SMILES: O=C(O)c1cc(-c2cccc(NC(=O)C3(c4ccc5c(c4)OC(F)(F)O5)CC3)n2)ccc1Cl] | 419 |
| 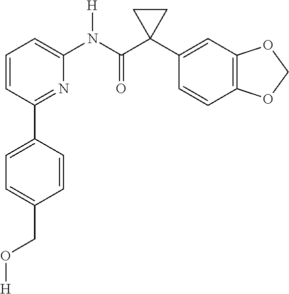 [SMILES: O=C(Nc1cccc(-c2ccc(CO)cc2)n1)C1(c2ccc3c(c2)OCO3)CC1] | 420 |
| 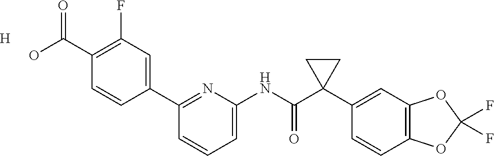 | 421 |
| 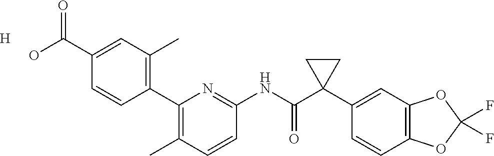 | 422 |
| 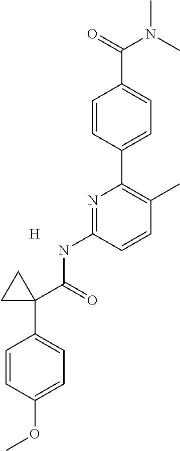 | 423 |
| 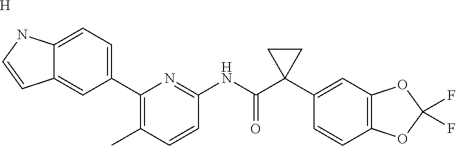 | 424 |
| 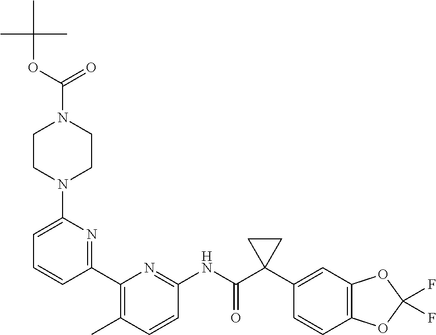 [SMILES: Cc1ccc(NC(=O)C2(c3ccc4c(c3)OC(F)(F)O4)CC2)nc1-c1cccc(N2CCN(C(=O)OC(C)(C)C)CC2)n1] | 425 |
| 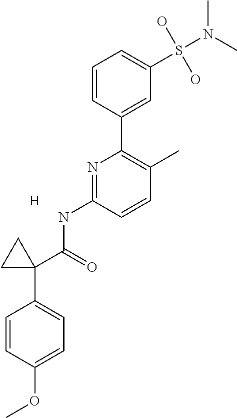 | 426 |
| 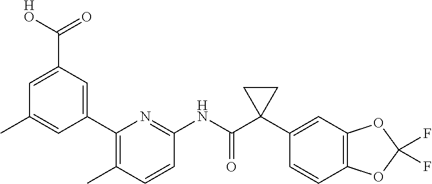 [SMILES: Cc1cc(C(=O)O)cc(-c2nc(NC(=O)C3(c4ccc5c(c4)OC(F)(F)O5)CC3)ccc2C)c1] | 427 |
| 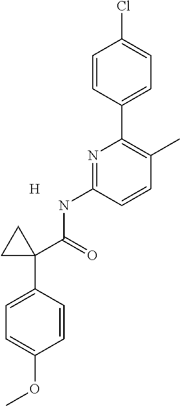 | 428 |
| 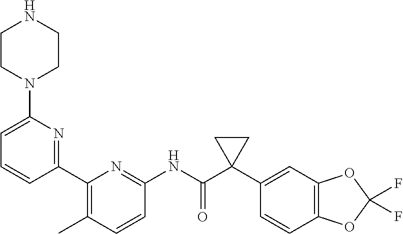 [SMILES: Cc1ccc(NC(=O)C2(c3ccc4c(c3)OC(F)(F)O4)CC2)nc1-c1cccc(N2CCNCC2)n1] | 429 |
| 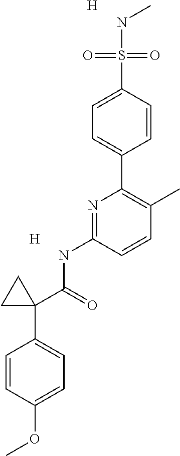 | 430 |
| 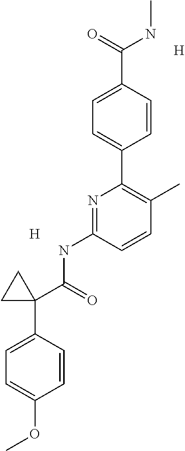 | 431 |
| 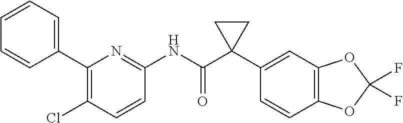 [SMILES: O=C(Nc1ccc(Cl)c(-c2ccccc2)n1)C1(c2ccc3c(c2)OC(F)(F)O3)CC1] | 432 |
| 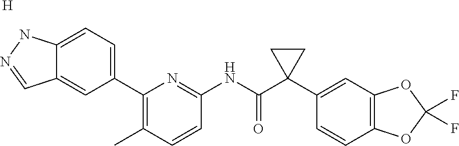 | 433 |
| 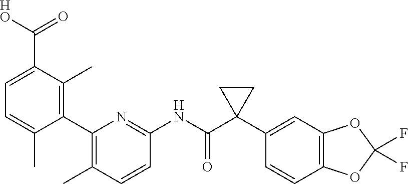 [SMILES: Cc1ccc(NC(=O)C2(c3ccc4c(c3)OC(F)(F)O4)CC2)nc1-c1c(C)ccc(C(=O)O)c1C] | 434 |
| 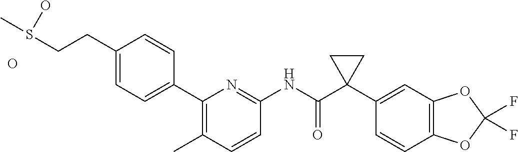 | 435 |
| 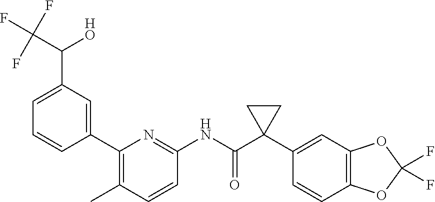 [SMILES: Cc1ccc(NC(=O)C2(c3ccc4c(c3)OC(F)(F)O4)CC2)nc1-c1cccc(C(O)C(F)(F)F)c1] | 436 |
| 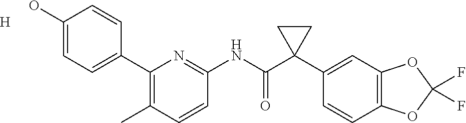 | 437 |
| 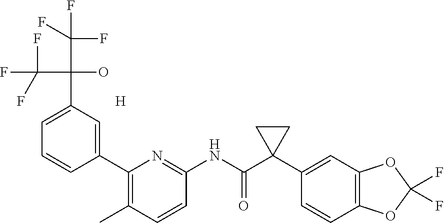 | 438 |
| 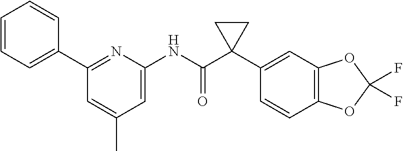 [SMILES: Cc1cc(NC(=O)C2(c3ccc4c(c3)OC(F)(F)O4)CC2)nc(-c2ccccc2)c1] | 439 |
| 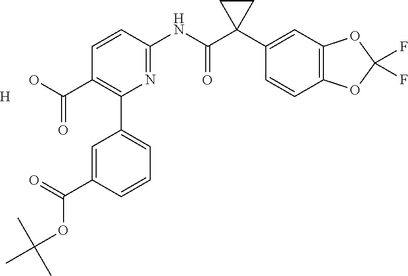 | 440 |
| 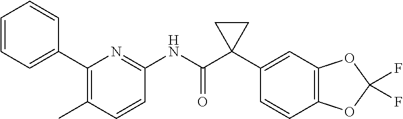 [SMILES: Cc1ccc(NC(=O)C2(c3ccc4c(c3)OC(F)(F)O4)CC2)nc1-c1ccccc1] | 441 |
| 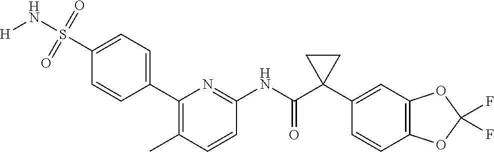 [SMILES: Cc1ccc(NC(=O)C2(c3ccc4c(c3)OC(F)(F)O4)CC2)nc1-c1ccc(S(N)(=O)=O)cc1] | 442 |
| 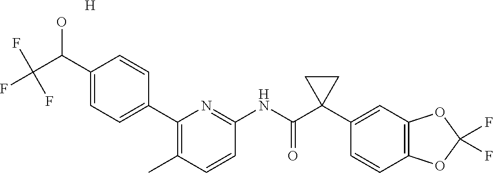 | 443 |
| 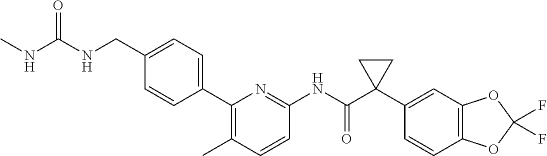 [SMILES: CNC(=O)NCc1ccc(-c2nc(NC(=O)C3(c4ccc5c(c4)OC(F)(F)O5)CC3)ccc2C)cc1] | 444 |
| 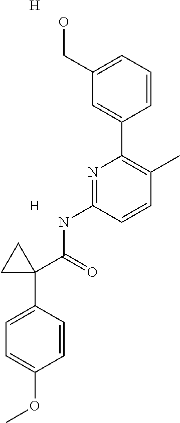 | 445 |
| 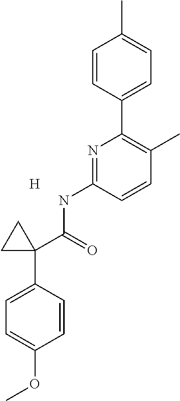 | 446 |
| 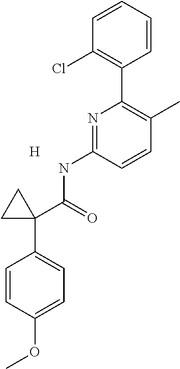 | 447 |
| 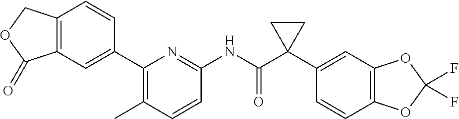 [SMILES: Cc1ccc(NC(=O)C2(c3ccc4c(c3)OC(F)(F)O4)CC2)nc1-c1ccc2c(c1)C(=O)OC2] | 448 |
| 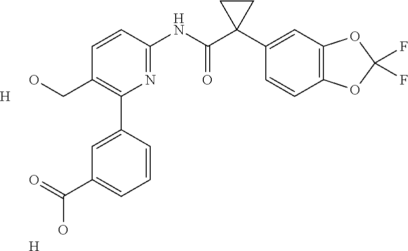 | 449 |
| 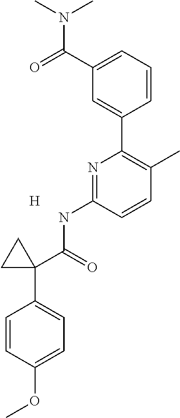 | 450 |
| 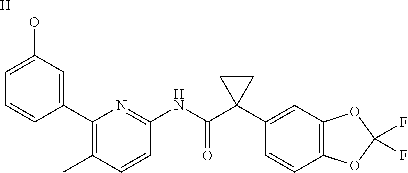 | 451 |
| 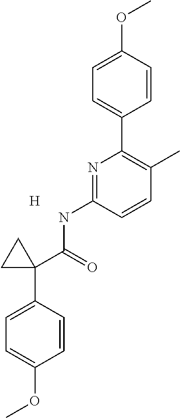 | 452 |
| 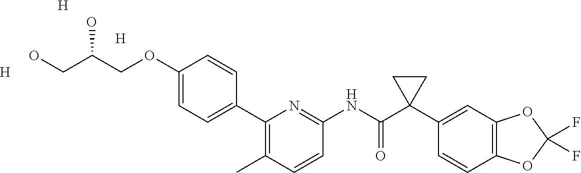 | 453 |
| 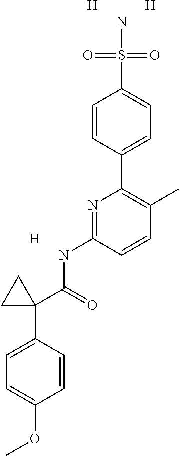 | 454 |
| 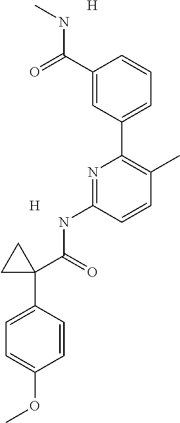 | 455 |
| 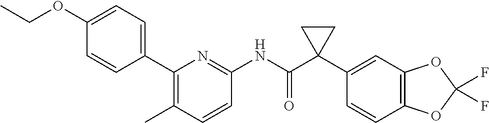 [SMILES: CCOc1ccc(-c2nc(NC(=O)C3(c4ccc5c(c4)OC(F)(F)O5)CC3)ccc2C)cc1] | 456 |
| 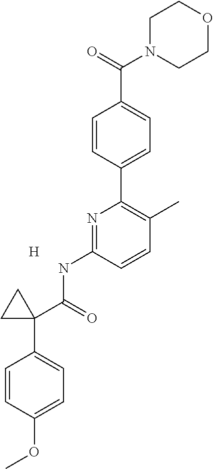 | 457 |
| 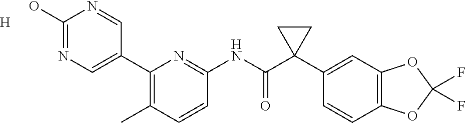 | 458 |
| 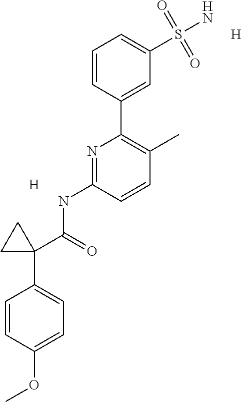 | 459 |
| 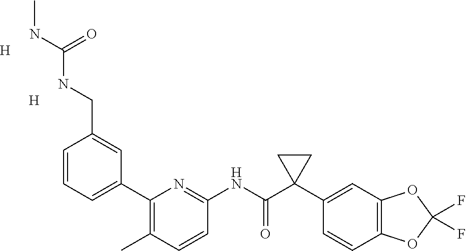 | 460 |
| 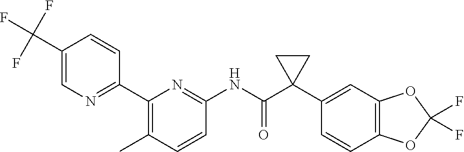 [SMILES: Cc1ccc(NC(=O)C2(c3ccc4c(c3)OC(F)(F)O4)CC2)nc1-c1ccc(C(F)(F)F)cn1] | 461 |
| 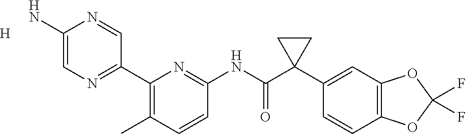 | 462 |
| 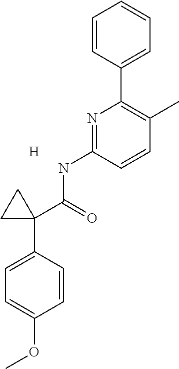 | 463 |
| 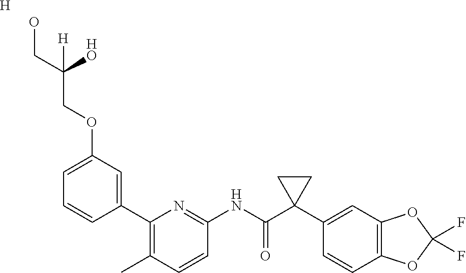 | 465 |
| 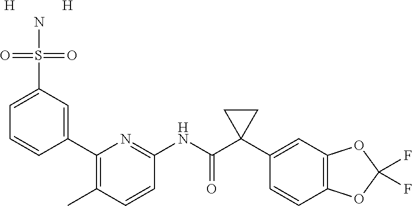 | 466 |
| 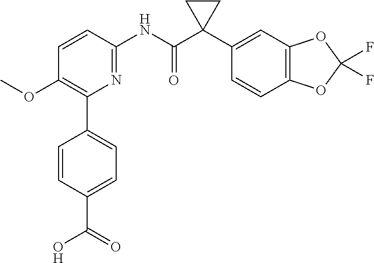 [SMILES: COc1ccc(NC(=O)C2(c3ccc4c(c3)OC(F)(F)O4)CC2)nc1-c1ccc(C(=O)O)cc1] | 467 |
| 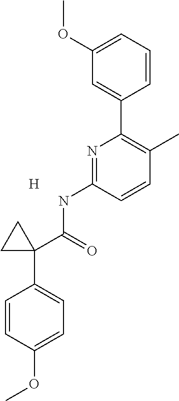 | 468 |
| 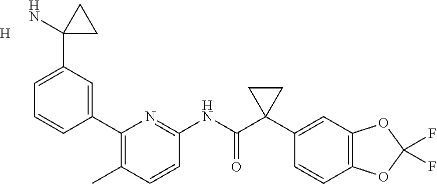 | 469 |
| 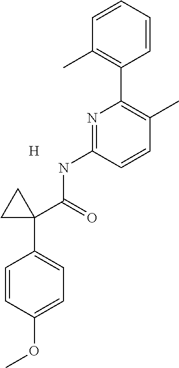 | 470 |
| 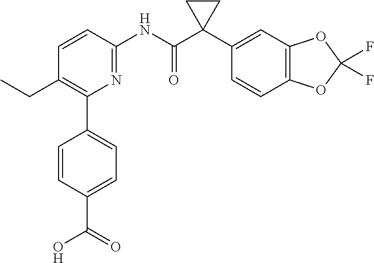 [SMILES: CCc1ccc(NC(=O)C2(c3ccc4c(c3)OC(F)(F)O4)CC2)nc1-c1ccc(C(=O)O)cc1] | 471 |
| 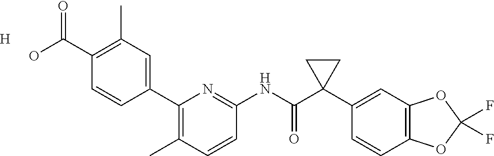 | 472 |
| 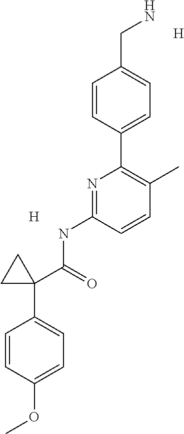 | 473 |
| 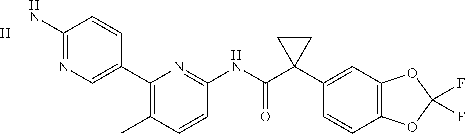 | 474 |
| 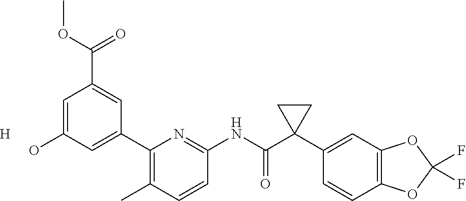 | 475 |
| 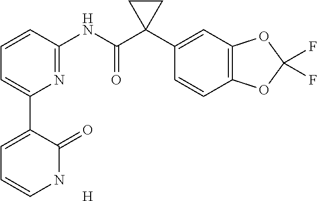 | 476 |
| 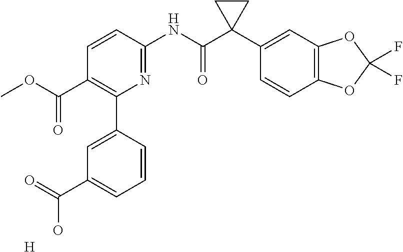 | 477 |
| 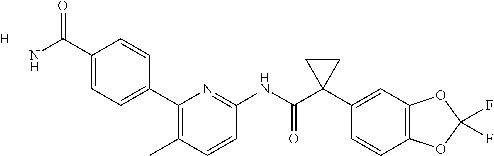 | 478 |
| 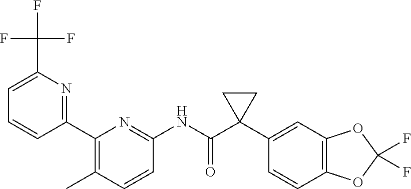 [SMILES: Cc1ccc(NC(=O)C2(c3ccc4c(c3)OC(F)(F)O4)CC2)nc1-c1cccc(C(F)(F)F)n1] | 479 |
| 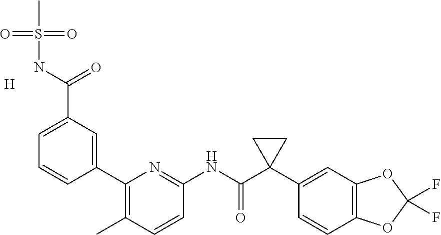 | 480 |
| 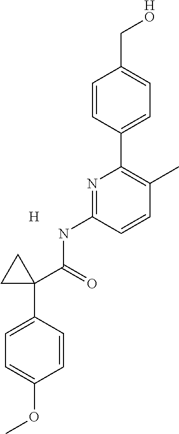 | 481 |
| 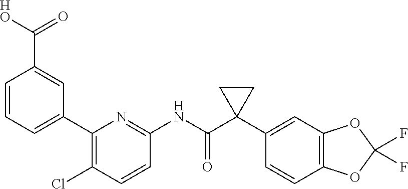 [SMILES: O=C(O)c1cccc(-c2nc(NC(=O)C3(c4ccc5c(c4)OC(F)(F)O5)CC3)ccc2Cl)c1] | 482 |
| 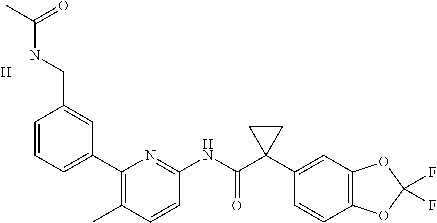 | 483 |
| 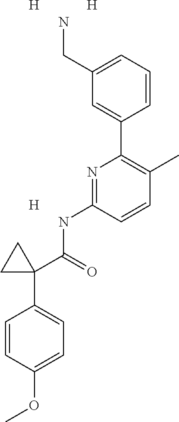 | 484 |
| 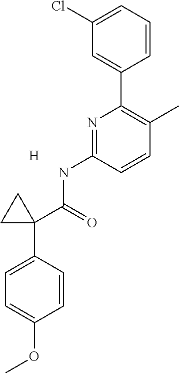 | 485 |
| 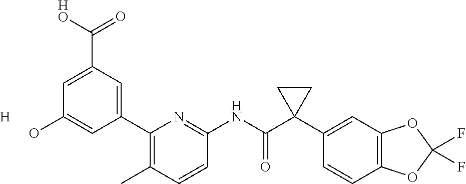 | 486 |
| 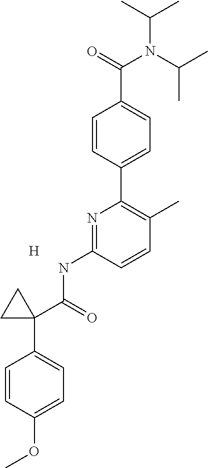 | 487 |
| 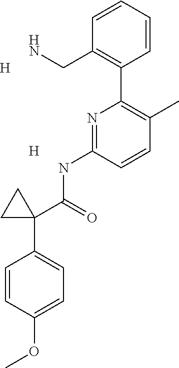 | 488 |
| 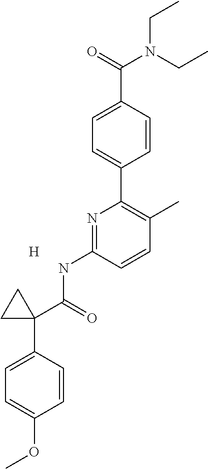 | 489 |
| 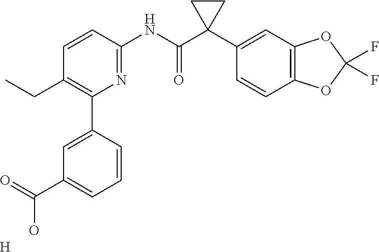 | 490 |
| 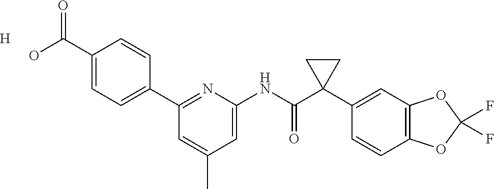 | 491 |
| 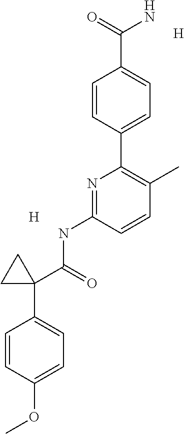 | 492 |
| 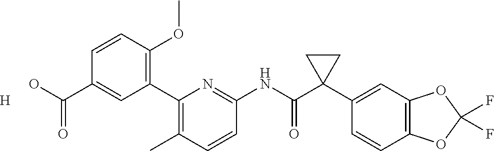 | 493 |
| 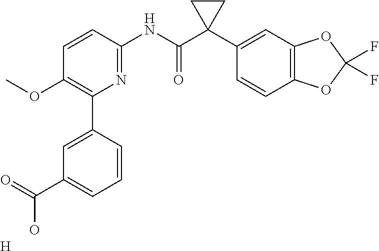 | 494 |
| 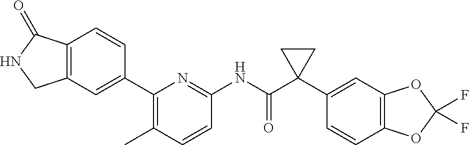 [SMILES: Cc1ccc(NC(=O)C2(c3ccc4c(c3)OC(F)(F)O4)CC2)nc1-c1ccc2c(c1)CNC2=O] | 495 |
| 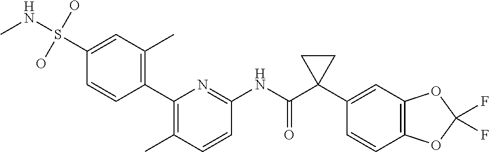 [SMILES: CNS(O)(O)c1ccc(-c2nc(NC(=O)C3(c4ccc5c(c4)OC(F)(F)O5)CC3)ccc2C)c(C)c1] | 496 |
| 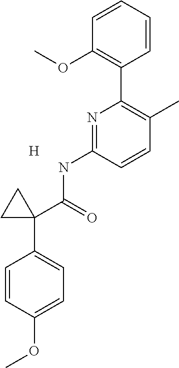 | 497 |
| 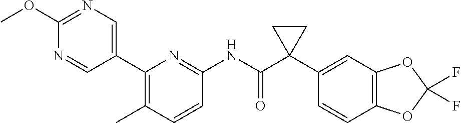 [SMILES: COc1ncc(-c2nc(NC(=O)C3(c4ccc5c(c4)OC(F)(F)O5)CC3)ccc2C)cn1] | 498 |
| 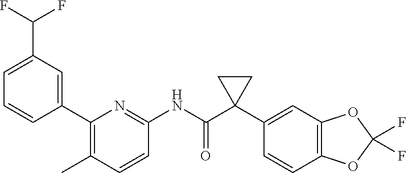 [SMILES: Cc1ccc(NC(=O)C2(c3ccc4c(c3)OC(F)(F)O4)CC2)nc1-c1cccc(C(F)F)c1] | 499 |
| 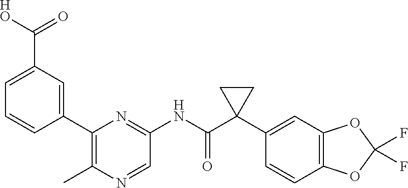 [SMILES: Cc1ncc(NC(=O)C2(c3ccc4c(c3)OC(F)(F)O4)CC2)nc1-c1cccc(C(=O)O)c1] | 500 |
| 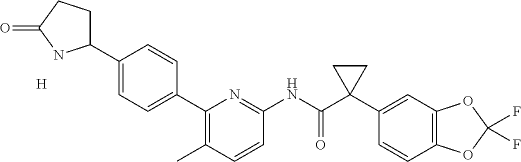 | 501 |
| 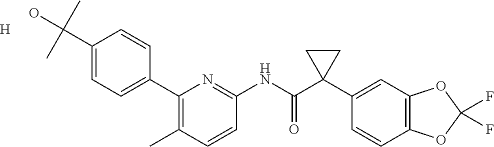 | 502 |
| 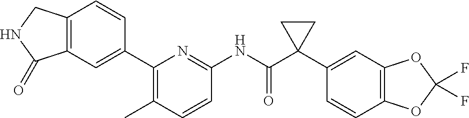 [SMILES: Cc1ccc(NC(=O)C2(c3ccc4c(c3)OC(F)(F)O4)CC2)nc1-c1ccc2c(c1)C(=O)NC2] | 503 |
| 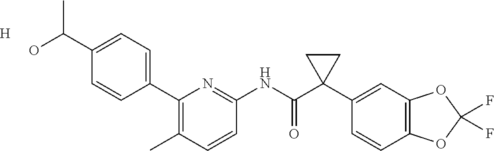 | 504 |
| 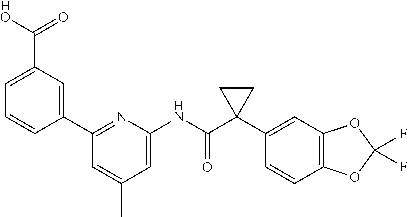 [SMILES: Cc1cc(NC(=O)C2(c3ccc4c(c3)OC(F)(F)O4)CC2)nc(-c2cccc(C(=O)O)c2)c1] | 505 |
| 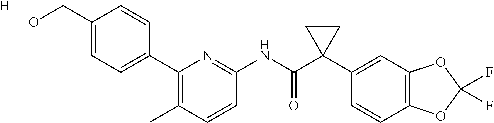 | 506 |
| 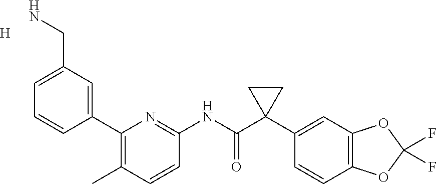 | 507 |
| 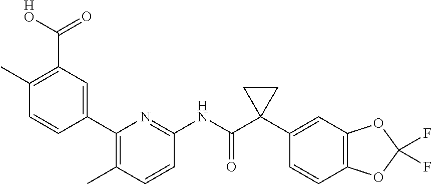 [SMILES: Cc1ccc(-c2nc(NC(=O)C3(c4ccc5c(c4)OC(F)(F)O5)CC3)ccc2C)cc1C(=O)O] | 508 |
| 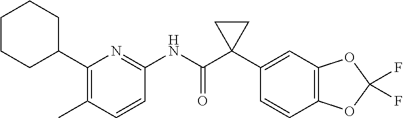 [SMILES: Cc1ccc(NC(=O)C2(c3ccc4c(c3)OC(F)(F)O4)CC2)nc1C1CCCCC1] | 509 |
| 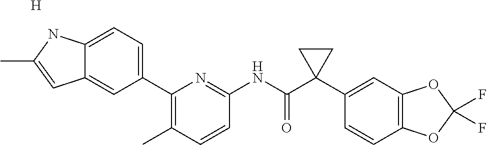 | 510 |
| 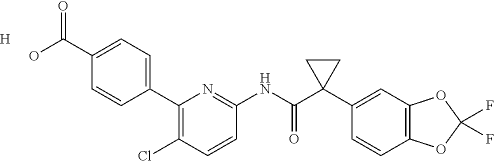 | 511 |
| 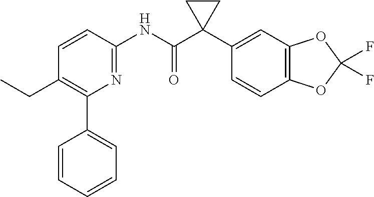 [SMILES: CCc1ccc(NC(=O)C2(c3ccc4c(c3)OC(F)(F)O4)CC2)nc1-c1ccccc1] | 512 |
| 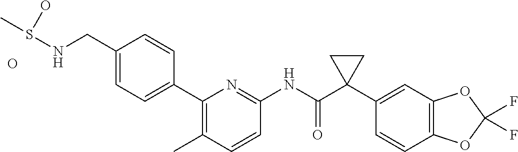 | 513 |
| 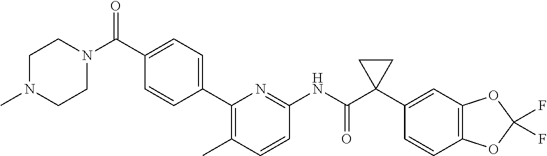 [SMILES: Cc1ccc(NC(=O)C2(c3ccc4c(c3)OC(F)(F)O4)CC2)nc1-c1ccc(C(=O)N2CCN(C)CC2)cc1] | 514 |
| 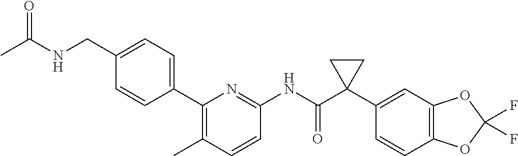 [SMILES: CC(=O)NCc1ccc(-c2nc(NC(=O)C3(c4ccc5c(c4)OC(F)(F)O5)CC3)ccc2C)cc1] | 515 |
| 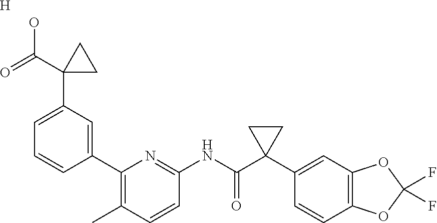 | 516 |
| 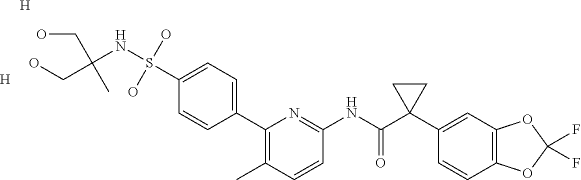 | 517 |
| 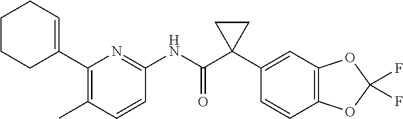 [SMILES: Cc1ccc(NC(=O)C2(c3ccc4c(c3)OC(F)(F)O4)CC2)nc1C1=CCCCC1] | 518 |
| 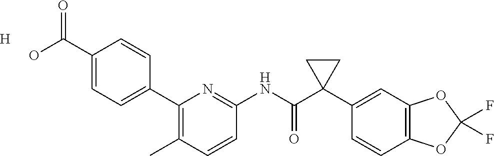 | 519 |
| 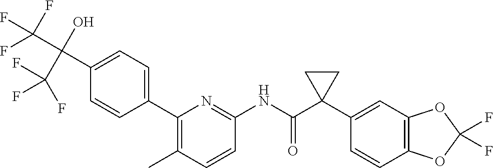 [SMILES: Cc1ccc(NC(=O)C2(c3ccc4c(c3)OC(F)(F)O4)CC2)nc1-c1ccc(C(O)(C(F)(F)F)C(F)(F)F)cc1] | 520 |
| 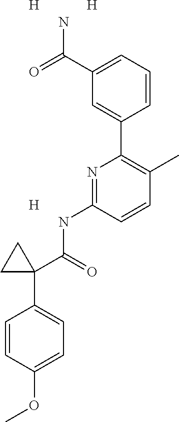 | 521 |
| 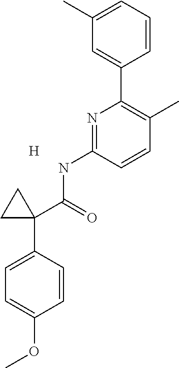 | 522 |
| 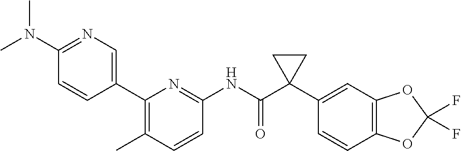 [SMILES: Cc1ccc(NC(=O)C2(c3ccc4c(c3)OC(F)(F)O4)CC2)nc1-c1ccc(N(C)C)nc1] | 523 |
| 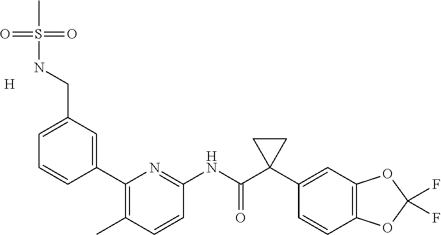 | 524 |
| 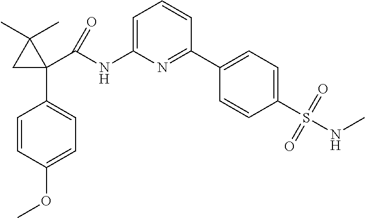 | 525 |
| 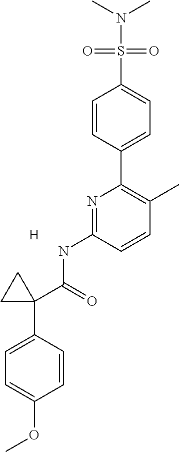 | 526 |
| 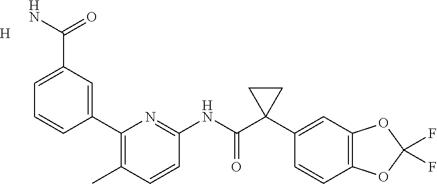 | 527 |
| 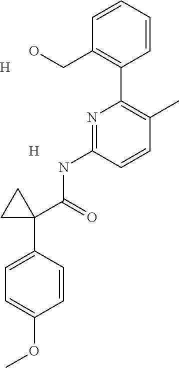 | 528 |

### IV. Synthetic Schemes

Compounds of the invention may be prepared by known methods or as illustrated in the examples. In one instance wherein CR₁ is aryl or heteroaryl, the compounds of the invention may be prepared as illustrated in Scheme I.

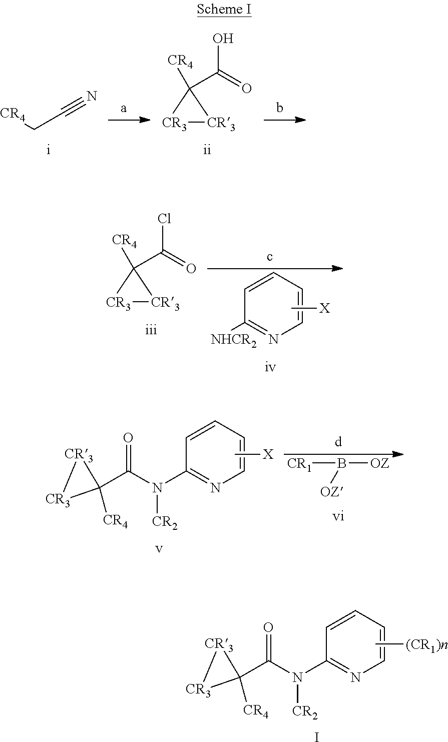

a) 50% NaOH, X—CR₃—CR′₃—Y, BTEAC; Y=leaving group; b) SOCl₂, DMF; c) pyridine; d) Pd(dppf)Cl₂, K₂CO₃, DMF, H₂O

Referring to Scheme I, a nitrile of formula i is alkylated (step a) with a dihalo-aliphatic in the presence of a base such as, for example, 50% sodium hydroxide and, optionally, a phase transfer reagent such as, for example, benzyltriethylammonium chloride (BTEAC), to produce the corresponding alkylated nitrile (not shown) which on hydrolysis produces the acid ii. Compounds of formula ii are converted to the acid chloride iii with a suitable reagent such as, for example, thionyl chloride/DMF. Reaction of the acid chloride iii with an aminopyridine, wherein X is a halo, of formula iv (step c) produces the amide of formula v. Reaction of the amide v with a boronic acid derivative vi (step d) wherein Z and Z′ are independently H, alkyl or Z and Z′ together with the atoms to which they are bound form a five or six membered optionally substituted cycloaliphatic ring, in the presence of a catalyst such as, for example, palladium acetate or dichloro-[1,1-bis(diphenylphosphino)ferrocene]palladium(II) (Pd(dppf)Cl₂), provides compounds of the invention wherein R₁ is aryl or heteroaryl. The boronic acid derivatives vi are commercially available or may be prepared by known methods such as reaction of an aryl bromide with a diborane ester in the presence of a coupling reagent such as, for example, palladium acetate as described in the examples.

In another instance where one CR₁ is aryl and another CR₁ is an aliphatic, alkoxy, cycloaliphatic, or heterocycloaliphatic, compounds of the invention can be prepared as described in steps a, b, and c of Scheme I using an appropriately substituted aminopyridine such as

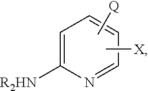

where X is halo and Q is C₁₋₆ aliphatic, aryl, heteroaryl, or 3 to 10 membered cycloaliphatic or heterocycloaliphatic as a substitute for the aminopyridine of formula iv.

### VI. Preparations and Examples

### General Procedure I

### Carboxylic Acid Building Block

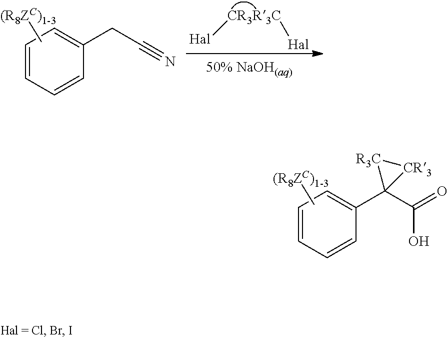

Benzyltriethylammonium chloride (0.025 equivalents) and the appropriate dihalo compound (2.5 equivalents) were added to a substituted phenyl acetonitrile. The mixture was heated to 70° C. and then 50% sodium hydroxide (10 equivalents) was slowly added to the mixture. The reaction was stirred at 70° C. for 12-24 hours to insure complete formation of the cycloalkyl moiety and then heated at 150° C. for 24-48 hours to insure complete conversion from the nitrile to the carboxylic acid. The dark brown/black reaction mixture was diluted with water and extracted with dichloromethane three times to remove side products. The basic aqueous solution was acidified with concentrated hydrochloric acid to pH less than one and the precipitate which began to form at pH 4 was filtered and washed with 1 M hydrochloric acid two times. The solid material was dissolved in dichloromethane and extracted two times with 1 M hydrochloric acid and one time with a saturated aqueous solution of sodium chloride. The organic solution was dried over sodium sulfate and evaporated to dryness to give the cycloalkylcarboxylic acid a white solid.

### Example I-1

### 1-Benzo[1,3]dioxol-5-yl-cyclopropanecarboxylic acid (A-1)

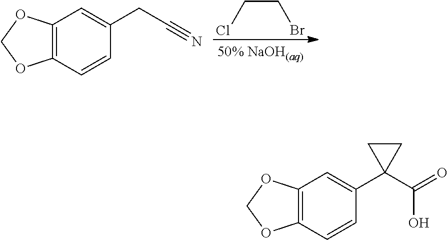

A mixture of benzo[1,3]dioxole-5-carbonitrile (5.10 g 31.7 mmol), 1-bromo-2-chloro-ethane (9.00 mL 109 mmol), and benzyltriethylammonium chloride (0.181 g, 0.795 mmol) was heated to 70° C. and then 50% (wt./wt.) aqueous sodium hydroxide (26 mL) was slowly added to the mixture. The reaction was stirred at 70° C. for 24 hours and then heated to 130° C. for 48 hours. The dark brown reaction mixture was diluted with water (400 mL) and extracted once with an equal volume of ethyl acetate and once with an equal volume of dichloromethane. The basic aqueous solution was acidified with concentrated hydrochloric acid to pH less than one and the precipitate filtered and washed with 1 M hydrochloric acid. The solid material was dissolved in dichloromethane (400 mL) and extracted twice with equal volumes of 1 M hydrochloric acid and once with a saturated aqueous solution of sodium chloride. The organic solution was dried over sodium sulfate and evaporated to dryness to give a white to slightly off-white solid. ESI-MS m/z calc. 206.1. found 207.1 (M+1)+. Retention time 2.37 minutes. ¹H NMR (400 MHz, DMSO-d₆) δ 1.07-1.11 (m, 2H), 1.38-1.42 (m, 2H), 5.98 (s, 2H), 6.79 (m, 2H), 6.88 (m, 1H), 12.26 (s, 1H).

### General Procedure II

### Carboxylic Acid Building Block

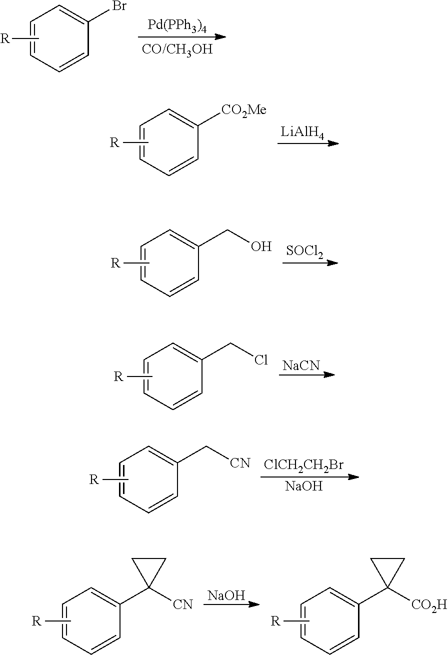

wherein R is —Z^{C}R₈.

### Example II-1

### 1-(2,2-Difluoro-benzo[1,3]dioxol-5-yl)-cyclopropanecarboxylic acid (A-2)

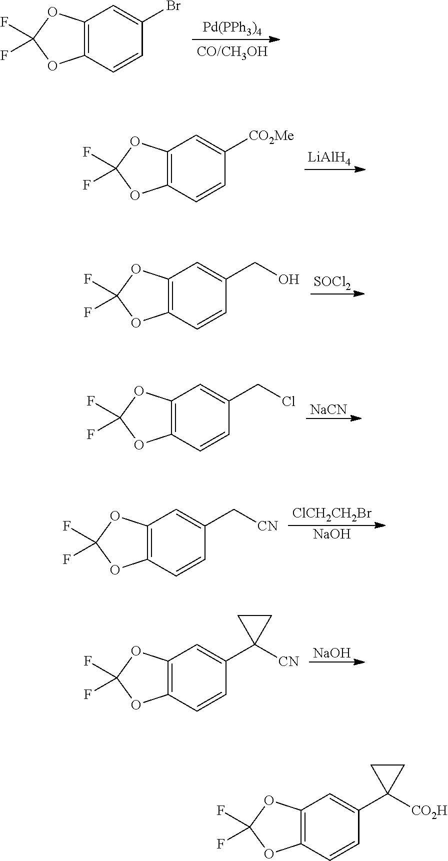

### Step a: 2,2-Difluoro-benzo[1,3]dioxole-5-carboxylic acid methyl ester

A solution of 5-bromo-2,2-difluoro-benzo[1,3]dioxole (11.8 g, 50.0 mmol) and tetrakis(triphenylphosphine)palladium (0) [Pd(PPh₃)₄, 5.78 g, 5.00 mmol] in methanol (20 mL) containing acetonitrile (30 mL) and triethylamine (10 mL) was stirred under a carbon monoxide atmosphere (55 PSI) at 75° C. (oil bath temperature) for 15 hours. The cooled reaction mixture was filtered and the filtrate was evaporated to dryness. The residue was purified by silica gel column chromatography to give crude 2,2-difluoro-benzo[1,3]dioxole-5-carboxylic acid methyl ester (11.5 g), which was used directly in the next step.

### Step b: (2,2-Difluoro-benzo[1,3]dioxol-5-yl)-methanol

Crude 2,2-Difluoro-benzo[1,3]dioxole-5-carboxylic acid methyl ester (11.5 g) dissolved in 20 mL of anhydrous tetrahydrofuran (THF) was slowly added to a suspension of lithium aluminum hydride (4.10 g, 106 mmol) in anhydrous THF (100 mL) at 0° C. The mixture was then warmed to room temperature. After being stirred at room temperature for 1 hour, the reaction mixture was cooled to 0° C. and treated with water (4.1 g), followed by sodium hydroxide (10% aqueous solution, 4.1 mL). The resulting slurry was filtered and washed with THF. The combined filtrate was evaporated to dryness and the residue was purified by silica gel column chromatography to give (2,2-difluoro-benzo[1,3]dioxol-5-yl)-methanol as a colorless oil.

### Step c: 5-Chloromethyl-2,2-difluoro-benzo[1,3]dioxole

Thionyl chloride (45 g, 38 mmol) was slowly added to a solution of (2,2-difluoro-benzo[1,3]dioxol-5-yl)-methanol (7.2 g, 38 mmol) in dichloromethane (200 mL) at 0° C. The resulting mixture was stirred overnight at room temperature and then evaporated to dryness. The residue was partitioned between an aqueous solution of saturated sodium bicarbonate (100 mL) and dichloromethane (100 mL). The separated aqueous layer was extracted with dichloromethane (150 mL) and the organic layer was dried over sodium sulfate, filtrated, and evaporated to dryness to give crude 5-chloromethyl-2,2-difluoro-benzo[1,3]dioxole which was used directly in the next step.

### Step d: (2,2-Difluoro-benzo[1,3]dioxol-5-yl)-acetonitrile

A mixture of crude 5-chloromethyl-2,2-difluoro-benzo[1,3]dioxole (4.4 g) and sodium cyanide (1.36 g, 27.8 mmol) in dimethylsulfoxide (50 mL) was stirred at room temperature overnight. The reaction mixture was poured into ice and extracted with ethyl acetate (300 mL). The organic layer was dried over sodium sulfate and evaporated to dryness to give crude (2,2-difluoro-benzo[1,3]dioxol-5-yl)-acetonitrile which was used directly in the next step.

### Step e: 1-(2,2-Difluoro-benzo[1,3]dioxol-5-yl)-cyclopropanecarbonitrile

Sodium hydroxide (50% aqueous solution, 10 mL) was slowly added to a mixture of crude (2,2-difluoro-benzo[1,3]dioxol-5-yl)-acetonitrile, benzyltriethylammonium chloride (3.00 g, 15.3 mmol), and 1-bromo-2-chloroethane (4.9 g, 38 mmol) at 70° C. The mixture was stirred overnight at 70° C. before the reaction mixture was diluted with water (30 mL) and extracted with ethyl acetate. The combined organic layers were dried over sodium sulfate and evaporated to dryness to give crude 1-(2,2-difluoro-benzo[1,3]dioxol-5-yl)-cyclopropanecarbonitrile, which was used directly in the next step.

### Step f: 1-(2,2-Difluoro-benzo[1,3]dioxol-5-yl)-cyclopropanecarboxylic acid (A-2)

1-(2,2-Difluoro-benzo[1,3]dioxol-5-yl)-cyclopropanecarbonitrile (crude from the last step) was refluxed in 10% aqueous sodium hydroxide (50 mL) for 2.5 hours. The cooled reaction mixture was washed with ether (100 mL) and the aqueous phase was acidified to pH 2 with 2M hydrochloric acid. The precipitated solid was filtered to give 1-(2,2-difluoro-benzo[1,3]dioxol-5-yl)-cyclopropanecarboxylic acid as a white solid. ESI-MS m/z calc. 242.04. found 241.58 (M+1)⁺; ¹H NMR (CDCl₃) δ 7.14-7.04 (m, 2H), 6.98-6.96 (m, 1H), 1.74-1.64 (m, 2H), 1.26-1.08 (m, 2H).

The following Table II.C-2 contains a list of carboxylic acid building blocks that were commercially available, or prepared by one of the two methods described above:

TABLE II
C-2: Carboxylic acid building blocks.

| Compound | Name |
|---|---|
| A-1 | 1-benzo[1,3]dioxol-5-ylcyclopropane-1-carboxylic acid |
| A-2 | 1-(2,2-difluorobenzo[1,3]dioxol-5-yl)cyclopropane-1- |
| | carboxylic acid |
| A-3 | 1-(3,4-dimethoxyphenyl)cyclopropane-1-carboxylic acid |
| A-4 | 1-(3-methoxyphenyl)cyclopropane-1-carboxylic acid |
| A-5 | 1-(2-methoxyphenyl)cyclopropane-1-carboxylic acid |
| A-6 | 1-[4-(trifluoromethoxy)phenyl]cyclopropane-1-carboxylic acid |
| A-7 | 1-(4-methylsulfanylphenyl)cyclopropane-1-carboxylic acid |
| A-8 | tetrahydro-4-(4-methoxyphenyl)-2H-pyran-4-carboxylic acid |
| A-9 | 1-phenylcyclopropane-1-carboxylic acid |
| A-10 | 1-(4-methoxyphenyl)cyclopropane-1-carboxylic acid |
| A-11 | 1-(4-chlorophenyl)cyclopropane-1-carboxylic acid |
| A-12 | 1-(p-tolyl)cyclopropane-1-carboxylic acid |
| A-13 | 1-phenylcyclopentanecarboxylic acid |
| A-14 | 1-phenylcyclohexanecarboxylic acid |
| A-15 | 1-(4-methoxyphenyl)cyclopentanecarboxylic acid |
| A-16 | 1-(4-methoxyphenyl)cyclohexanecarboxylic acid |
| A-17 | 1-(4-chlorophenyl)cyclohexanecarboxylic acid |
| A-18 | 1-(2,3-dihydrobenzo[b][1,4]dioxin-7- |
| | yl)cyclopropanecarboxylic acid |
| A-19 | 1-(4H-benzo[d][1,3]dioxin-7-yl)cyclopropanecarboxylic acid |
| A-20 | 1-(2,2,4,4-tetrafluoro-4H-benzo[d][1,3]dioxin-6-yl) |
| | cyclopropanecarboxylic acid |
| A-21 | 1-(4H-benzo[d][1,3]dioxin-6-yl)cyclopropanecarboxylic acid |
| A-22 | 1-(quinoxalin-7-yl)cyclopropanecarboxylic acid |
| A-23 | 1-(quinolin-6-yl)cyclopropanecarboxylic acid |
| A-24 | 1-(4-chlorophenyl)cyclopentanecarboxylic acid |

### General Procedure III

### Coupling Reactions

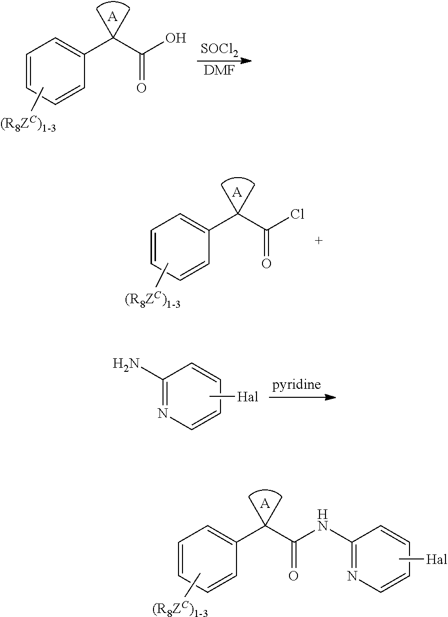 [1381] Hal=Cl, Br, I, all other variables. Ring A is the ring formed by R₃ and R′₃.

One equivalent of the appropriate carboxylic acid was placed in an oven-dried flask under nitrogen. Thionyl chloride (3 equivalents) and a catalytic amount of and N,N-dimethylformamide was added and the solution was allowed to stir at 60° C. for 30 minutes. The excess thionyl chloride was removed under vacuum and the resulting solid was suspended in a minimum of anhydrous pyridine. This solution was slowly added to a stirred solution of one equivalent the appropriate aminoheterocycle dissolved in a minimum of anhydrous pyridine. The resulting mixture was allowed to stir for 15 hours at 110° C. The mixture was evaporated to dryness, suspended in dichloromethane, and then extracted three times with 1N NaOH. The organic layer was then dried over sodium sulfate, evaporated to dryness, and then purified by column chromatography.

### Example III-1

### 1-Benzo[1,3]dioxol-5-yl-cyclopropanecarboxylic acid [5-(2-chloro-benzoyl)-thiazol-2-yl]-amide (B-1)

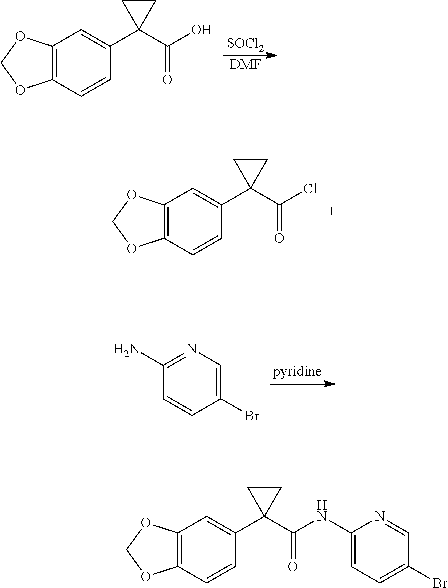

1-Benzo[1,3]dioxol-5-yl-cyclopropanecarboxylic acid (2.38 g, 11.5 mmol) was placed in an oven-dried flask under nitrogen. Thionyl chloride (2.5 mL) and N,N-dimethylformamide (0.3 mL) were added and the solution was allowed to stir for 30 minutes at 60° C. The excess thionyl chloride was removed under vacuum and the resulting solid was suspended in 7 mL of anhydrous pyridine. This solution was then slowly added to a solution of 5-bromo-pyridin-2-ylamine (2.00 g, 11.6 mmol) suspended in 10 mL of anhydrous pyridine. The resulting mixture was allowed to stir for 15 hours at 110° C. The mixture was then evaporated to dryness, suspended in 100 mL of dichloromethane, and washed with three 25 mL portions of 1N NaOH. The organic layer was dried over sodium sulfate, evaporated to near dryness, and then purified by silica gel column chromatography utilizing dichloromethane as the eluent to yield the pure product (3.46 g, 83%) ESI-MS m/z calc. 359.0. found 361.1 (M+1)+; Retention time 3.40 minutes. ¹H NMR (400 MHz, DMSO-d₆) δ 1.06-1.21 (m, 2H), 1.44-1.51 (m, 2H), 6.07 (s, 2H), 6.93-7.02 (m, 2H), 7.10 (d, J=1.6 Hz, 1H), 8.02 (d, J=1.6 Hz, 2H), 8.34 (s, 1H), 8.45 (s, 1H)

### Example III-2

### 1-(Benzo[d][1,3]dioxol-6-yl)-N-(6-bromopyridin-2-yl)cyclopropanecarboxamide (B-2)

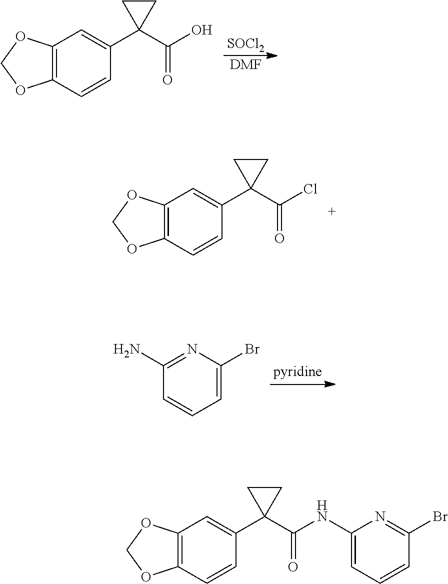

(1-Benzo[1,3]dioxol-5-yl-cyclopropanecarboxylic acid (1.2 g, 5.8 mmol) was placed in an oven-dried flask under nitrogen. Thionyl chloride (2.5 mL) and N,N-dimethylformamide (0.3 mL) were added and the solution was allowed to stir at 60° C. for 30 minutes. The excess thionyl chloride was removed under vacuum and the resulting solid was suspended in 5 mL of anhydrous pyridine. This solution was then slowly added to a solution of 6-bromopyridin-2-amine (1.0 g, 5.8 mmol) suspended in 10 mL of anhydrous pyridine. The resulting mixture was allowed to stir for 15 hours at 110° C. The mixture was then evaporated to dryness, suspended in 50 mL of dichloromethane, and washed with three 20 mL portions of 1N NaOH. The organic layer was dried over sodium sulfate, evaporated to near dryness, and then purified by silica gel column chromatography utilizing dichloromethane containing 2.5% triethylamine as the eluent to yield the pure product. ESI-MS m/z calc. 360.0. found 361.1 (M+1)⁺; Retention time 3.43 minutes. ¹H NMR (400 MHz, DMSO-d₆): δ 1.10-1.17 (m, 2H), 1.42-1.55 (m, 2H), 6.06 (s, 2H), 6.92-7.02 (m, 2H), 7.09 (d, J=1.6 Hz, 1H), 7.33 (d, J=7.6 Hz, 1H), 7.73 (t, J=8.0 Hz, 1H), 8.04 (d, J=8.2 Hz, 1H), 8.78 (s, 1H).

The compounds in the following Table II.C-3 were prepared in a manner analogous to that described above:

TABLE II.C-3
Exemplary compounds synthesized according to example III.

| | | Retention | | ¹H NMR |
|---|---|---|---|---|
| Compound | Name | Time (min) | (M + 1)⁺ | (400 MHz, DMSO-d₆) |
| B-3 | 1-(benzo[d][1,3]dioxol-5- | 3.58 | 375.3 | ¹H NMR (400 MHz, |
| | yl)-N-(5-bromo-6- | | | DMSO) δ 8.39 (s, 1H), |
| | methylpyridin-2- | | | 7.95 (d, J = 8.7 Hz, |
| | yl)cyclopropanecarboxamide | | | 1H), 7.83 (d, J = 8.8 Hz, |
| | | | | 1H), 7.10 (d, J = 1.6 Hz, |
| | | | | 1H), |
| | | | | 7.01-6.94 (m, 2H), 6.06 (s, |
| | | | | 2H), 2.41 (s, 3H), |
| | | | | 1.48-1.46 (m, 2H), |
| | | | | 1.14-1.10 (m, 2H) |
| B-4 | 1-(benzo[d][1,3]dioxol-5- | 2.90 | 331.0 | |
| | yl)-N-(6-chloro-5- | | | |
| | methylpyridin-2- | | | |
| | yl)cyclopropanecarboxamide | | | |
| B-5 | 1-(benzo[d][1,3]dioxol-5- | 3.85 | 375.1 | ¹H NMR (400 MHz, |
| | yl)-N-(5-bromo-4- | | | DMSO) δ 8.36 (s, 1H), |
| | methylpyridin-2- | | | 8.30 (s, 1H), 8.05 (s, |
| | yl)cyclopropanecarboxamide | | | 1H), 7.09 (d, J = 1.6 Hz, |
| | | | | 1H), |
| | | | | 7.01-6.95 (m, 2H), 6.07 (s, 2H), |
| | | | | 2.35 (s, 3H), |
| | | | | 1.49-1.45 (m, 2H), |
| | | | | 1.16-1.13 (m, 2H) |
| B-6 | 1-(benzo[d][1,3]dioxol-5- | 3.25 | 389.3 | |
| | yl)-N-(5-bromo-6- | | | |
| | methylpyridin-2- | | | |
| | yl)cyclopropanecarboxamide | | | |
| B-7 | 1-(benzo[d][1,3]dioxol-5- | 2.91 | 375.1 | |
| | yl)-N-(5-bromo-3- | | | |
| | methylpyridin-2- | | | |
| | yl)cyclopropanecarboxamide | | | |

### General Procedure IV

### Compounds of Formula C

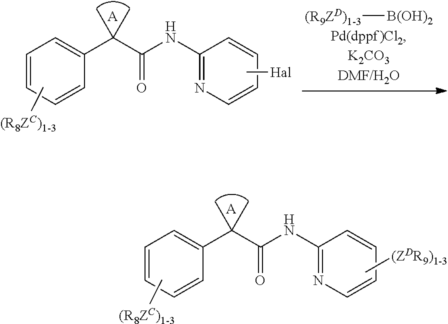 [1389] Hal=Cl, Br, I. Ring A is the ring formed by R₃ and R′₃.

The appropriate aryl halide (1 equivalent) was dissolved in 1 mL of N,N-dimethylformamide (DMF) in a reaction tube. The appropriate boronic acid (1.3 equivalents), 0.1 mL of an aqueous 2 M potassium carbonate solution (2 equivalents), and a catalytic amount of Pd(dppf)Cl₂ (0.09 equivalents) were added and the reaction mixture was heated at 80° C. for three hours or at 150° C. for 5 min in the microwave. The resulting material was cooled to room temperature, filtered, and purified by reverse-phase preparative liquid chromatography.

### Example IV-1

### 1-Benzo[1,3]dioxol-5-yl-cyclopropanecarboxylic acid [5-(2,4-dimethoxy-phenyl)-pyridin-2-yl]-amide

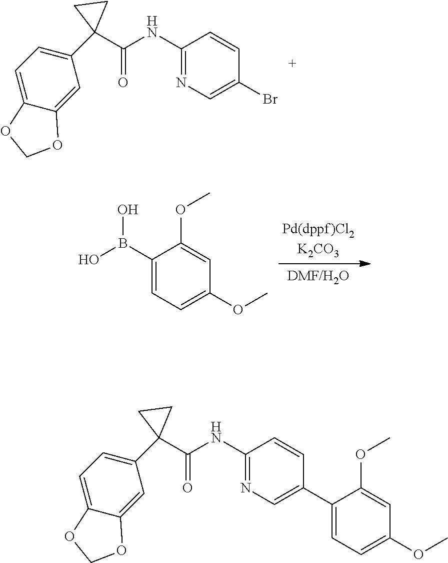

1-Benzo[1,3]dioxol-5-yl-cyclopropanecarboxylic acid (5-bromo-pyridin-2-yl)-amide (36.1 mg, 0.10 mmol) was dissolved in 1 mL of N,N-dimethylformamide in a reaction tube. 2,4-Dimethoxybenzeneboronic acid (24 mg, 0.13 mmol), 0.1 mL of an aqueous 2 M potassium carbonate solution, and a catalytic amount of Pd(dppf)Cl₂ (6.6 mg, 0.0090 mmol) were added and the reaction mixture was heated to 80° C. for three hours. The resulting material was cooled to room temperature, filtered, and purified by reverse-phase preparative liquid chromatography to yield the pure product as a trifluoroacetic acid salt. ESI-MS m/z calc. 418.2. found 419.0 (M+1)⁺. Retention time 3.18 minutes. ¹H NMR (400 MHz, CD₃CN) δ 1.25-1.29 (m, 2H), 1.63-1.67 (m, 2H), 3.83 (s, 3H), 3.86 (s, 3H), 6.04 (s, 2H), 6.64-6.68 (m, 2H), 6.92 (d, J=8.4 Hz, 1H), 7.03-7.06 (m, 2H), 7.30 (d, J=8.3 Hz, 1H), 7.96 (d, J=8.9 Hz, 1H), 8.14 (dd, J=8.9, 2.3 Hz, 1H), 8.38 (d, J=2.2 Hz, 1H), 8.65 (s, 1H).

### Example IV-2

### 1-Benzo[1,3]dioxol-5-yl-cyclopropanecarboxylic acid [6-(4-dimethylamino-phenyl)-pyridin-2-yl]-amide

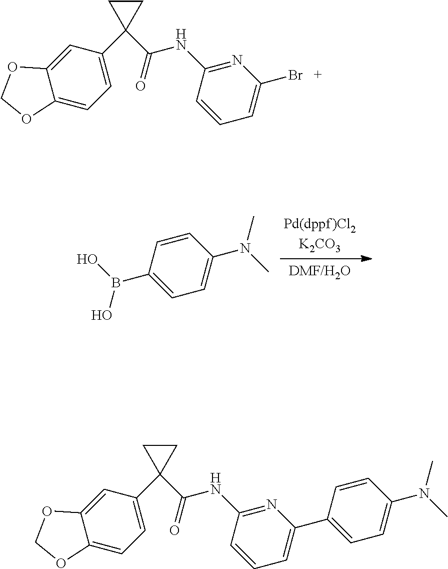

1-Benzo[1,3]dioxol-5-yl-cyclopropanecarboxylic acid (6-bromo-pyridin-2-yl)-amide (36 mg, 0.10 mmol) was dissolved in 1 mL of N,N-dimethylformamide in a reaction tube. 4-(Dimethylamino)phenylboronic acid (21 mg, 0.13 mmol), 0.1 mL of an aqueous 2 M potassium carbonate solution, and (Pd(dppf)Cl₂ (6.6 mg, 0.0090 mmol) were added and the reaction mixture was heated at 80° C. for three hours. The resulting material was cooled to room temperature, filtered, and purified by reverse-phase preparative liquid chromatography to yield the pure product as a trifluoroacetic acid salt. ESI-MS m/z calc. 401.2. found 402.5 (M+1)⁺. Retention time 2.96 minutes. ¹H NMR (400 MHz, CD₃CN) δ 1.23-1.27 (m, 2H), 1.62-1.66 (m, 2H), 3.04 (s, 6H), 6.06 (s, 2H), 6.88-6.90 (m, 2H), 6.93-6.96 (m, 1H), 7.05-7.07 (m, 2H), 7.53-7.56 (m, 1H), 7.77-7.81 (m, 3H), 7.84-7.89 (m, 1H), 8.34 (s, 1H).

### General Procedure V

The following schemes were utilized to prepare additional boronic esters which were not commercially available:

### Specific Example V-1

### 1-Methyl-4-[4-(4,4,5,5-tetramethyl-1,3,2-dioxaborolan-2-yl)phenyl]sulfonyl-piperazine

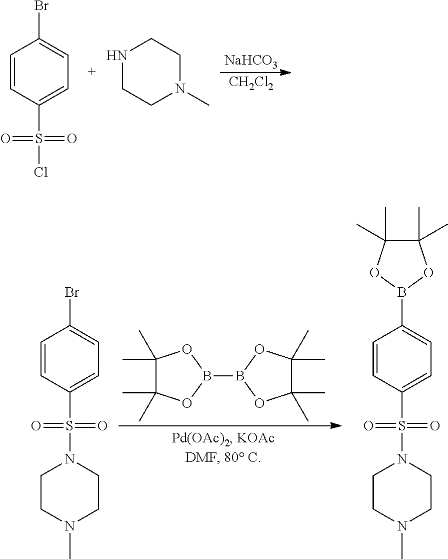

### Step a: 1-(4-Bromophenylsulfonyl)-4-methylpiperazine

A solution of 4-bromobenzene-1-sulfonyl chloride (256 mg, 1.00 mmol) in 1 mL of dichloromethane was slowly added to a vial (40 mL) containing 5 mL of a saturated aqueous solution of sodium bicarbonate, dichloromethane (5 mL) and 1-methylpiperazine (100 mg, 1.00 mmol). The reaction was stirred at room temperature overnight. The phases were separated and the organic layer was dried over magnesium sulfate. Evaporation of the solvent under reduced pressure provided the required product, which was used in the next step without further purification. ESI-MS m/z calc. 318.0. found 318.9 (M+1)⁺. Retention time of 1.30 minutes. ¹H NMR (300 MHz, CDCl₃) δ 7.65 (d, J=8.7 Hz, 2H), 7.58 (d, J=8.7 Hz, 2H), 3.03 (t, J=4.2 Hz, 4H), 2.48 (t, J=4.2 Hz, 4H), 2.26 (s, 3H).

### Step b: 1-Methyl-4-[4-(4,4,5,5-tetramethyl-1,3,2-dioxaborolan-2-yl)phenyl]sulfonyl-piperazine

A 50 mL round bottom flask was charged with 1-(4-bromophenylsulfonyl)-4-methylpiperazine (110 mg, 0.350 mmol), bis-(pinacolato)-diboron (93 mg, 0.37 mmol), palladium acetate (6 mg, 0.02 mmol), and potassium acetate (103 mg, 1.05 mmol) in N,N-dimethylformamide (6 mL). The mixture was degassed by gently bubbling argon through the solution for 30 minutes at room temperature. The mixture was then heated at 80° C. under argon until the reaction was complete (4 hours). The required product, 1-methyl-4-[4-(4,4,5,5-tetramethyl-1,3,2-dioxaborolan-2-yl)phenyl]sulfonyl-piperazine, and the bi-aryl product, 4-(4-methylpiperazin-1-ylsulfonyl)phenyl-phenylsulfonyl-4-methylpiperazine, were obtained in a ratio of 1:2 as indicated by LC/MS analysis.

### Specific Example V-2

### tert-Butyl 4-(4,4,5,5-tetramethyl-1,3,2-dioxaborolan-2-yl)benzylmethylcarbamate

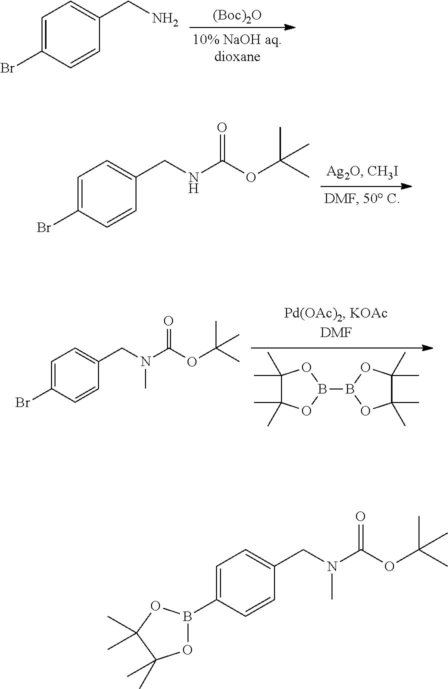

### Step a: tert-Butyl-4-bromobenzylcarbamate

Commercially available p-bromobenzylamine hydrochloride (1 g, 4 mmol) was treated with 10% aq. NaOH (5 mL). To the clear solution was added (Boc)2O (1.1 g, 4.9 mmol) dissolved in dioxane (10 mL). The mixture was vigorously stirred at room temperature for 18 hours. The resulting residue was concentrated, suspended in water (20 mL), extracted with ethyl acetate (4×20 mL), dried over Na₂SO₄, filtered, and concentrated to yield tert-butyl-4-bromobenzylcarbamate as a white solid. ¹H NMR (300 MHz, DMSO-d₆) δ 7.48 (d, J=8.4 Hz, 2H), 7.40 (t, J=6 Hz, 1H), 7.17 (d, J=8.4 Hz, 2H), 4.07 (d, J=6.3 Hz, 2H), 1.38 (s, 9H).

### Step b: tert-Butyl-4-bromobenzyl(methyl)carbamate

In a 60-mL vial, tert-butyl-4-bromobenzylcarbamate (1.25 g, 4.37 mmol) was dissolved in DMF (12 mL). To this solution was added Ag₂O (4.0 g, 17 mmol) followed by the addition of CH₃I (0.68 mL, 11 mmol). The mixture was stirred at 50° C. for 18 hours. The reaction mixture was filtered through a bed of celite and the celite was washed with methanol (2×20 mL) and dichloromethane (2×20 mL). The filtrate was concentrated to remove most of the DMF. The residue was treated with water (50 mL) and a white emulsion formed. This mixture was extracted with ethyl acetate (4×25 mL), dried over Na₂SO₄, and the solvent was evaporated to yield tert-butyl-4-bromobenzyl(methyl)carbamate as a yellow oil. ¹H NMR (300 MHz, DMSO-d₆) δ 7.53 (d, J=8.1 Hz, 2H), 7.15 (d, J=8.4 Hz, 2H), 4.32 (s, 2H), 2.74 (s, 3H), 1.38 (s, 9H).

### Step c: tert-Butyl 4-(4,4,5,5-tetramethyl-1,3,2-dioxaborolan-2-yl)benzylmethylcarbamate

The coupling reaction was achieved in the same manner as described above for 1-methyl-4-[4-(4,4,5,5-tetramethyl-1,3,2-dioxaborolan-2-yl)phenyl]sulfonyl-piperazine, Preparation 3. The protecting Boc group was removed after the coupling reaction by treating the crude reaction mixture with 0.5 mL of 1N HCl in diethyl ether for 18 hours before purification by HPLC.

### Specific Example V-3

### 4,4,5,5-Tetramethyl-2-(4-(2-(methylsulfonyl)ethyl)phenyl)-1,3,2-dioxaborolane

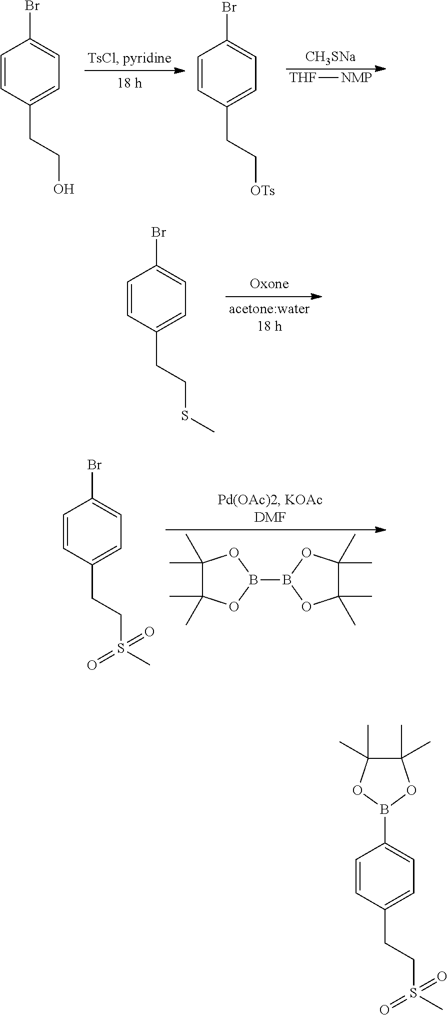

### Step a: 4-Bromophenethyl-4-methylbenzenesulfonate

To a 50 mL round-bottom flask was added p-bromophenethyl alcohol (1.0 g, 4.9 mmol), followed by the addition of pyridine (15 mL). To this clear solution was added, under argon, p-toluenesulfonyl chloride (TsCl) (1.4 g, 7.5 mmol) as a solid. The reaction mixture was purged with Argon and stirred at room temperature for 18 hours. The crude mixture was treated with 1N HCl (20 mL) and extracted with ethyl acetate (5×25 mL). The organic fraction was dried over Na₂SO₄, filtered, and concentrated to yield 4-bromophenethyl-4-methylbenzenesulfonate as a yellowish liquid. ¹H-NMR (Acetone-d₆, 300 MHz) δ 7.64 (d, J=8.4 Hz, 2H), 7.40-7.37 (d, J=8.7 Hz, 4H), 7.09 (d, J=8.5 Hz, 2H), 4.25 (t, J=6.9 Hz, 2H), 2.92 (t, J=6.3 Hz, 2H), 2.45 (s, 3H).

### Step b: (4-Bromophenethyl)(methyl)sulfane

To a 20 mL round-bottom flask were added 4-bromophenethyl 4-methylbenzenesulfonate (0.354 g, 0.996 mmol) and CH₃SNa (0.10 g, 1.5 mmol), followed by the addition of tetrahydrofuran (THF) (1.5 mL) and N-methyl-2-pynolidinone (NMP) (1.0 mL). The mixture was stirred at room temperature for 48 hours before it was treated with a saturated aqueous solution of sodium bicarbonate (10 mL). The mixture was extracted with ethyl acetate (4×10 mL), dried over Na₂SO₄, filtered, and concentrated to yield (4-bromophenethyl)(methyl)sulfane as a yellowish oil. ¹H-NMR (CDCl₃, 300 MHz) δ 7.40 (d, J=8.4 Hz, 2H), 7.06 (d, J=8.4 Hz, 2H), 2.89-2.81 (m, 2H), 2.74-2.69 (m, 2H), 2.10 (s, 3H).

### Step c: 1-Bromo-4-(2-methylsulfonyl)-ethylbenzene

To a 20 mL round-bottom flask were added (4-bromophenethyl)(methyl)sulfane (0.311 g, 1.34 mmol) and Oxone (3.1 g, 0.020 mol), followed by the addition of a 1:1 mixture of acetone/water (10 mL). The mixture was vigorously stirred at room temperature for 20 hours, before the volatiles were removed. The aqueous mixture was extracted with ethyl acetate (3×15 mL) and dichloromethane (DCM) (3×10 mL). The organic fractions were combined, dried with Na₂SO₄, filtered, and concentrated to yield a white semisolid. Purification of the crude material by flash chromatography yielded 1-bromo-4-(2-methylsulfonyl)-ethylbenzene. ¹H-NMR (DMSO-d₆, 300 MHz) δ 7.49 (d, J=8.4 Hz, 2H), 7.25 (d, J=8.7 Hz, 2H), 3.43 (m, 2H), 2.99 (m, 2H), 2.97 (s, 3H).

### Step d: 4,4,5,5-Tetramethyl-2-(4-(2-(methylsulfonyl)ethyl)phenyl)-1,3,2-dioxaborolane

The coupling reaction was achieved in the same manner as described above for 1-methyl-4-[4-(4,4,5,5-tetramethyl-1,3,2-dioxaborolan-2-yl)phenyl]sulfonyl-piperazine, step b.

The compounds in the following table were synthesized as described above using commercially available or previously described boronic acids:

### General Procedure VI

### Compound Derivatization after Coupling

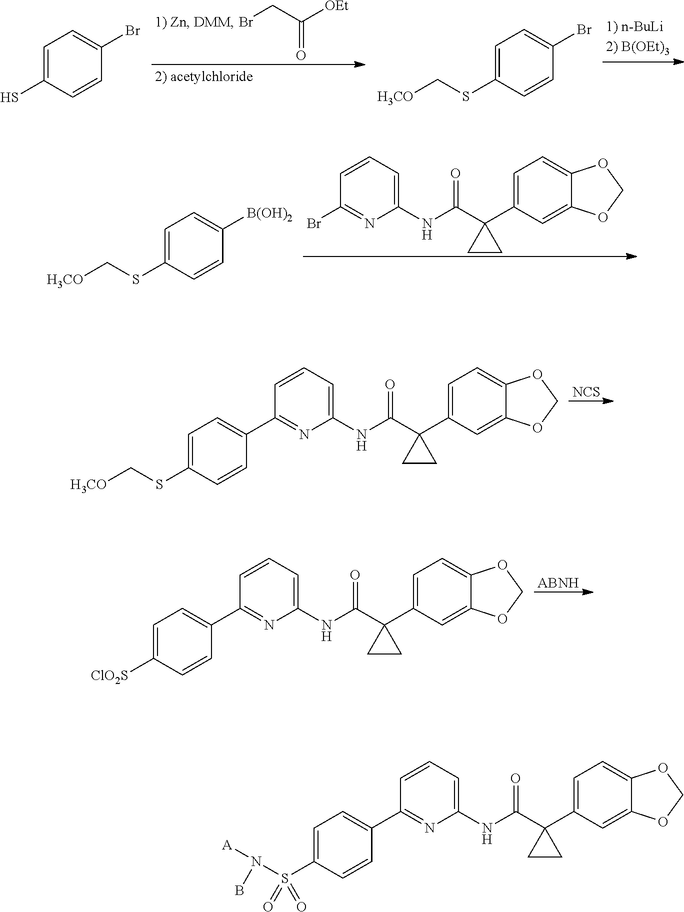

### Step a: (4-Bromophenyl)(methoxymethyl)sulfane

To a mixture of Zn (3.25 g, 50 mmol) in 10 mL dimethoxymethane was added a few drops of ethyl bromoacetate. A mixture of ethyl bromoacetate (8.35 g, 50 mmol) in 20 mL dimethoxymethane was added dropwise maintaining the temperature between 30° C. and 35° C. The mixture was then heated at reflux for an additional hour before 4-bromothiophenol (7.56 g, 40 mmol) in 10 mL dimethoxymethane was added drop-wise. The mixture was heated at reflux for 3 hours and cooled to −5° C. Acetylchloride (2.5 g, 2.27 mL, 40 mmol) was added dropwise (T<0° C.) and the mixture was stirred at RT overnight. A mixture of 25% aq. NH₃/sat. aq. NH₄Cl solution (100 mL, 1:1) was added before the mixture was extracted with TBME (2×). The combined organic layers were washed with brine, dried (NaSO₄) and the solvent was evaporated to give (4-bromophenyl)(methoxymethyl)sulfane as a yellow oil. ¹H NMR (300 MHz, CDCl₃) δ 3.42 (s, 3H), 4.92 (s, 2H), 7.31-7.36 (d, 2H), 7.40-7.45 (d, 2H).

### Step b: 4-(Methoxymethylthio)phenylboronic acid

A mixture of (4-bromophenyl)(methoxymethyl)sulfane (5.7 g, 24.4 mmol) in 25 mL THF was cooled to −78° C. n-BuLi (15.7 mL, 2.5M in hexanes, 1.6 eq.) was added dropwise (T<−78° C.) and the mixture was stirred at −78° C. for 15 minutes. Triethylborate (20.8 mL, 5 eq.) was added dropwise (T<−78° C.) and the mixture was stirred at −78° C. for 2 hours and then at RT for 4 days. Water was added and the organic solvents were evaporated. The mixture was extracted with TBME (2×). The combined organic layers were washed with brine, dried (NaSO₄) and the solvent was evaporated to give a light brown oil. The product was purified by column chromatography (SiO₂, EtOAc/Heptane 1:3) to give 4-(methoxymethylthio)phenylboronic acid as a light yellow solid. ¹H NMR (300 MHz, CDCl₃) δ 3.46 (s, 3H), 5.08 (s, 2H), 7.54-7.58 (d, 2H), 8.08-8.12 (d, 2H).

### Step c: 1-(Benzo[d][1,3]dioxol-5-yl)-N-(6-(4-(methoxymethylthio)phenyl)pyridin-2-yl)cyclopropanecarboxamide

A mixture of 1-(benzo[d][1,3]dioxol-6-yl)-N-(6-bromopyridin-2-yl)cyclopropanecarboxamide (85 mg, 0.235 mmol) and 4-(methoxymethylthio)phenylboronic acid (53 mg, 1.2 eq.) was dissolved in 5 mL dioxane before sat. aq. HaHCO₃ solution (1 mL) was added followed by Pd(PPh₃)₄ (30 mg). The mixture was stirred at 90° C. for 4 hours (NMR indicated s.m.) and then at reflux overnight. The mixture was cooled and sat. aq. NaHCO₃ was added followed by EtOAc. The layers were separated and the aq. layer was extracted with EtOAc. The combined organic layers were washed with brine, dried (Na₂SO₄) and the solvent was evaporated to give a yellow oil. This oil was purified by column chromatography (SiO₂, EtOAc/heptane 1:15) to give 1-(benzo[d][1,3]dioxol-6-yl)-N-(6-(4-(methoxymethylthio)phenyl)pyridin-2-yl)cyclopropanecarboxamide as white solid. ¹H NMR (300 MHz, CDCl₃) δ 1.12-1.16 (m, 2H), 1.64-1.70 (m, 2H), 3.41 (s, 3H), 4.98 (s, 2H), 6.02 (s, 2H), 6.84-6.86 (d, 1H), 6.96-7.00 (m, 2H), 7.37-7.41 (d, 1H), 7.47-7.53 (m, 2H), 7.68-7.73 (t, 1H), 7.76-7.81 (m, 2H), 7.82-7.88 (br, 1H), 8.10-8.13 (d, 1H).

### Specific Example VI-1

### 1-Benzo[1,3]dioxol-5-yl-N-[6-[4-[(methyl-methylsulfonyl-amino)methyl]phenyl]-2-pyridyl]-cyclopropane-1-carboxamide

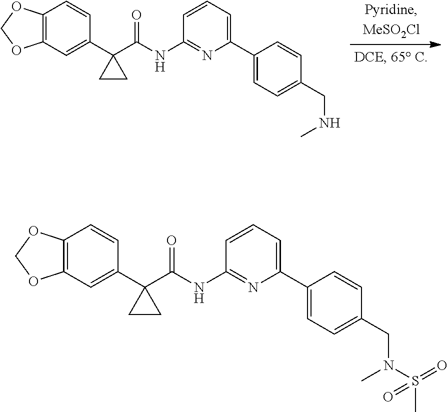 [SMILES: CNCc1ccc(-c2cccc(NC(=O)C3(c4ccc5c(c4)OCO5)CC3)n2)cc1 ; CN(Cc1ccc(-c2cccc(NC(=O)C3(c4ccc5c(c4)OCO5)CC3)n2)cc1)S(C)(=O)=O]

To the starting amine (brown semi-solid, 0.100 g, ˜0.2 mmol, obtained by treatment of the corresponding t-butyloxycarbonyl derivative by treatment with 1N HCl in ether) was added dichloroethane (DCE) (1.5 mL), followed by the addition of pyridine (0.063 mL, 0.78 mmol) and methansulfonyl chloride (0.03 mL, 0.4 mmol). The mixture was stirred at 65° C. for 3 hours. After this time, LC/MS analysis showed 50% conversion to the desired product. Two additional equivalents of pyridine and 1.5 equivalents of methansulfonyl chloride were added and the reaction was stirred for 2 hours. The mixture was concentrated then purified by HPLC to yield 1-benzo[1,3]dioxol-5-yl-N-[6-[4-[(methyl-methylsulfonyl-amino)methyl]phenyl]-2-pyridyl]-cyclopropane-1-carboxamide as a white solid. ESI-MS m/z calc. 479.2. found 480.1 (M+1)⁺.

Additional exemplary compounds of the present invention are illustrated in Table II.C-4:

TABLE II.C-4
Additional exemplary compounds of Formula C.

| Compound No. | Amine | Boronic Acid |
|---|---|---|
| 1 | B-2 | [2-(dimethylaminomethyl)phenyl]boronic acid |
| 2 | B-2 | [4-(1-piperidyl)phenyl]boronic acid |
| 3 | B-2 | (3,4-dichlorophenyl)boronic acid |
| 4 | B-2 | (4-morpholinosulfonylphenyl)boronic acid |
| 5 | B-2 | (3-chloro-4-methoxy-phenyl)boronic acid |
| 6 | B-2 | (6-methoxy-3-pyridyl)boronic acid |
| 7 | B-2 | (4-dimethylaminophenyl)boronic acid |
| 8 | B-2 | (4-morpholinophenyl)boronic acid |
| 9 | B-2 | [4-(acetylaminomethyl)phenyl]boronic acid |
| 10 | B-2 | (2-hydroxyphenyl)boronic acid |
| 11 | B-1 | 2-dihydroxyboranylbenzoic acid |
| 12 | B-1 | (6-methoxy-3-pyridyl)boronic acid |
| 13 | B-2 | 1-methyl-4-[4-(4,4,5,5-tetramethyl-1,3,2-dioxaborolan-2- |
| | | yl)phenyl]sulfonyl-piperazine |
| 14 | B-2 | (2,4-dimethylphenyl)boronic acid |
| 15 | B-2 | [3-(hydroxymethyl)phenyl]boronic acid |
| 16 | B-2 | 3-dihydroxyboranylbenzoic acid |
| 17 | B-2 | (3-ethoxyphenyl)boronic acid |
| 18 | B-2 | (3,4-dimethylphenyl)boronic acid |
| 19 | B-1 | [4-(hydroxymethyl)phenyl]boronic acid |
| 20 | B-1 | 3-pyridylboronic acid |
| 21 | B-2 | (4-ethylphenyl)boronic acid |
| 22 | B-2 | 2,6-dimethyl-4-[4-(4,4,5,5-tetramethyl-1,3,2-dioxaborolan-2- |
| | | yl)phenyl]sulfonyl-morpholine |
| 23 | B-2 | 4,4,5,5-tetramethyl-2-(4-(2-(methylsulfonyl)ethyl)phenyl)- |
| | | 1,3,2-dioxaborolane |
| 24 | B-1 | benzo[1,3]dioxol-5-ylboronic acid |
| 25 | B-2 | (3-chlorophenyl)boronic acid |
| 26 | B-2 | (3-methylsulfonylaminophenyl)boronic acid |
| 27 | B-2 | (3,5-dichlorophenyl)boronic acid |
| 28 | B-2 | (3-methoxyphenyl)boronic acid |
| 29 | B-1 | (3-hydroxyphenyl)boronic acid |
| 30 | B-2 | [1-[4-(4,4,5,5-tetramethyl-1,3,2-dioxaborolan-2- |
| | | yl)phenyl]sulfonyl-3-piperidyl]methanol |
| 31 | B-2 | phenylboronic acid |
| 32 | B-2 | (2,5-difluorophenyl)boronic acid |
| 33 | B-3 | phenylboronic acid |
| 34 | B-2 | N-(2-hydroxyethyl)-N-methyl-4-(4,4,5,5-tetramethyl-1,3,2- |
| | | dioxaborolan-2-yl)-benzenesulfonamide |
| 35 | B-2 | [(R)-1-[4-(4,4,5,5-tetramethyl-1,3,2-dioxaborolan-2- |
| | | yl)phenyl]sulfonylpyrrolidin-2-yl]methanol |
| 36 | B-2 | (2-methylsulfonylaminophenyl)boronic acid |
| 37 | B-1 | 1H-indol-5-ylboronic acid |
| 38 | B-2 | [4-[(2,2,2-trifluoroacetyl)aminomethyl]phenyl]boronic acid |
| 39 | B-2 | (2-chlorophenyl)boronic acid |
| 40 | B-1 | m-tolylboronic acid |
| 41 | B-2 | (2,4-dimethoxypyrimidin-5-yl)boronic acid |
| 42 | B-2 | (4-methoxycarbonylphenyl)boronic acid |
| 43 | B-2 | tert-butyl 4-(4,4,5,5-tetramethyl-1,3,2-dioxaborolan-2- |
| | | yl)benzylmethylcarbamate |
| 44 | B-2 | (4-ethoxyphenyl)boronic acid |
| 45 | B-2 | (3-methylsulfonylphenyl)boronic acid |
| 46 | B-2 | (4-fluoro-3-methyl-phenyl)boronic acid |
| 47 | B-2 | (4-cyanophenyl)boronic acid |
| 48 | B-1 | (2,5-dimethoxyphenyl)boronic acid |
| 49 | B-1 | (4-methylsulfonylphenyl)boronic acid |
| 50 | B-1 | cyclopent-1-enylboronic acid |
| 51 | B-2 | o-tolylboronic acid |
| 52 | B-1 | (2,6-dimethylphenyl)boronic acid |
| 53 | B-3 | 2-chlorophenylboronic acid |
| 54 | B-2 | (2,5-dimethoxyphenyl)boronic acid |
| 55 | B-2 | (2-fluoro-3-methoxy-phenyl)boronic acid |
| 56 | B-2 | (2-methoxyphenyl)boronic acid |
| 57 | B-7 | phenylboronic acid |
| 58 | B-2 | (4-isopropoxyphenyl)boronic acid |
| 59 | B-2 | (4-carbamoylphenyl)boronic acid |
| 60 | B-2 | (3,5-dimethylphenyl)boronic acid |
| 61 | B-2 | (4-isobutylphenyl)boronic acid |
| 62 | B-1 | (4-cyanophenyl)boronic acid |
| 63 | B-5 | phenylboronic acid |
| 64 | B-2 | N-ethyl-4-(4,4,5,5-tetramethyl-1,3,2-dioxaborolan-2-yl)- |
| | | benzenesulfonamide |
| 65 | B-1 | 2,3-dihydrobenzofuran-5-ylboronic acid |
| 66 | B-2 | (4-chlorophenyl)boronic acid |
| 67 | B-2 | (4-chloro-3-methyl-phenyl)boronic acid |
| 68 | B-2 | (2-fluorophenyl)boronic acid |
| 69 | B-2 | benzo[1,3]dioxol-5-ylboronic acid |
| 70 | B-2 | (4-morpholinocarbonylphenyl)boronic acid |
| 71 | B-1 | cyclohex-1-enylboronic acid |
| 72 | B-2 | (3,4,5-trimethoxyphenyl)boronic acid |
| 73 | B-2 | [4-(dimethylaminomethyl)phenyl]boronic acid |
| 74 | B-2 | m-tolylboronic acid |
| 75 | B-2 | N-(2-pyrrolidin-1-ylethyl)-4-(4,4,5,5-tetramethyl-1,3,2- |
| | | dioxaborolan-2-yl)-benzenesulfonamide |
| 76 | B-2 | 1-[4-(4,4,5,5-tetramethyl-1,3,2-dioxaborolan-2- |
| | | yl)phenyl]sulfonylpyrrolidine |
| 77 | B-2 | (3-cyanophenyl)boronic acid |
| 78 | B-2 | [3-(tert-butoxycarbonylaminomethyl)phenyl]boronic acid |
| 79 | B-2 | (4-methylsulfonylphenyl)boronic acid |
| 80 | B-1 | p-tolylboronic acid |
| 81 | B-2 | (2,4-dimethoxyphenyl)boronic acid |
| 82 | B-2 | (2-methoxycarbonylphenyl)boronic acid |
| 83 | B-2 | (2,4-difluorophenyl)boronic acid |
| 84 | B-2 | (4-isopropylphenyl)boronic acid |
| 85 | B-2 | [4-(2-dimethylaminoethylcarbamoyl)phenyl]boronic acid |
| 86 | B-1 | (2,4-dimethoxyphenyl)boronic acid |
| 87 | B-1 | benzofuran-2-ylboronic acid |
| 88 | B-2 | 2,3-dihydrobenzofuran-5-ylboronic acid |
| 89 | B-2 | (3-fluoro-4-methoxy-phenyl)boronic acid |
| 90 | B-2 | 1-[4-(4,4,5,5-tetramethyl-1,3,2-dioxaborolan-2- |
| | | yl)phenyl]sulfonylpiperidine |
| 91 | B-1 | (3-cyanophenyl)boronic acid |
| 92 | B-1 | (4-dimethylaminophenyl)boronic acid |
| 93 | B-2 | (2,6-dimethoxyphenyl)boronic acid |
| 94 | B-2 | (2-methoxy-5-methyl-phenyl)boronic acid |
| 95 | B-2 | (3-acetylaminophenyl)boronic acid |
| 96 | B-1 | (2,4-dimethoxypyrimidin-5-yl)boronic acid |
| 97 | B-2 | (5-fluoro-2-methoxy-phenyl)boronic acid |
| 98 | B-1 | [3-(hydroxymethyl)phenyl]boronic acid |
| 99 | B-1 | (2-methoxyphenyl)boronic acid |
| 100 | B-2 | (2,4,6-trimethylphenyl)boronic acid |
| 101 | B-2 | [4-(dimethylcarbamoyl)phenyl]boronic acid |
| 102 | B-2 | [4-(tert-butoxycarbonylaminomethyl)phenyl]boronic acid |
| 103 | B-2 | N-(tetrahydrofuran-2-ylmethyl)-4-(4,4,5,5-tetramethyl-1,3,2- |
| | | dioxaborolan-2-yl)-benzenesulfonamide |
| 104 | B-1 | (2-chlorophenyl)boronic acid |
| 105 | B-1 | (3-acetylaminophenyl)boronic acid |
| 106 | B-2 | (2-ethoxyphenyl)boronic acid |
| 107 | B-2 | 3-furylboronic acid |
| 108 | B-2 | [2-(hydroxymethyl)phenyl]boronic acid |
| 109 | B-2 | 1-[4-(4,4,5,5-tetramethyl-1,3,2-dioxaborolan-2- |
| | | yl)phenyl]sulfonylpiperidin-4-ol |
| 110 | B-7 | 2-chlorophenylboronic acid |
| 111 | B-2 | (2-fluoro-6-methoxy-phenyl)boronic acid |
| 112 | B-2 | (2-ethoxy-5-methyl-phenyl)boronic acid |
| 113 | B-2 | 1H-indol-5-ylboronic acid |
| 114 | B-1 | (3-chloro-4-pyridyl)boronic acid |
| 115 | B-2 | cyclohex-1-enylboronic acid |
| 116 | B-1 | o-tolylboronic acid |
| 117 | B-2 | [4-(tert-butylsulfamoyl)phenyl]boronic acid |
| 118 | B-2 | N-cyclopentyl-4-(4,4,5,5-tetramethyl-1,3,2-dioxaborolan-2- |
| | | yl)-benzenesulfonamide |
| 119 | B-2 | (2-aminophenyl)boronic acid |
| 120 | B-2 | (4-methoxy-3,5-dimethyl-phenyl)boronic acid |
| 121 | B-2 | (4-methoxyphenyl)boronic acid |
| 122 | B-2 | (2-propoxyphenyl)boronic acid |
| 123 | B-2 | (2-isopropoxyphenyl)boronic acid |
| 124 | B-2 | (2,3-dichlorophenyl)boronic acid |
| 125 | B-2 | (S)-2-(methoxymethyl)-1-[4-(4,4,5,5-tetramethyl-1,3,2- |
| | | dioxaborolan-2-yl)phenyl]sulfonyl-pyrrolidine |
| 126 | B-2 | (2,3-dimethylphenyl)boronic acid |
| 127 | B-2 | (4-fluorophenyl)boronic acid |
| 128 | B-1 | (3-methoxyphenyl)boronic acid |
| 129 | B-2 | (4-chloro-2-methyl-phenyl)boronic acid |
| 130 | B-1 | (2,6-dimethoxyphenyl)boronic acid |
| 131 | B-2 | (5-isopropyl-2-methoxy-phenyl)boronic acid |
| 132 | B-2 | (3-isopropoxyphenyl)boronic acid |
| 133 | B-2 | (R)-2-(methoxymethyl)-1-[4-(4,4,5,5-tetramethyl-1,3,2- |
| | | dioxaborolan-2-yl)phenyl]sulfonyl-pyrrolidine |
| 134 | B-2 | 4-dihydroxyboranylbenzoic acid |
| 135 | B-2 | (4-dimethylamino-2-methoxy-phenyl)boronic acid |
| 136 | B-2 | (4-methylsulfinylphenyl)boronic acid |
| 137 | B-2 | [4-(methylcarbamoyl)phenyl]boronic acid |
| 138 | B-1 | 8-quinolylboronic acid |
| 139 | B-2 | cyclopent-1-enylboronic acid |
| 140 | B-2 | p-tolylboronic acid |
| 141 | B-2 | [1-[4-(4,4,5,5-tetramethyl-1,3,2-dioxaborolan-2- |
| | | yl)phenyl]sulfonyl-4-piperidyl]methanol |
| 142 | B-3 | 2-methoxyphenylboronic acid |
| 143 | B-2 | (2,5-dimethylphenyl)boronic acid |
| 144 | B-1 | (3,4-dimethoxyphenyl)boronic acid |
| 145 | B-1 | (3-chlorophenyl)boronic acid |
| 146 | B-2 | [4-(morpholinomethyl)phenyl]boronic acid |
| 147 | B-5 | 4-(dimethylamino)phenylboronic acid |
| 148 | B-2 | [4-(methylsulfamoyl)phenyl]boronic acid |
| 149 | B-1 | 4-dihydroxyboranylbenzoic acid |
| 150 | B-1 | phenylboronic acid |
| 151 | B-2 | (2,3-difluorophenyl)boronic acid |
| 152 | B-1 | (4-chlorophenyl)boronic acid |
| 153 | B-7 | 2-methoxyphenylboronic acid |
| 154 | B-2 | 3-dihydroxyboranylbenzoic acid |
| 155 | B-5 | 2-methoxyphenylboronic acid |
| 156 | B-2 | N-methyl-N-propyl-4-(4,4,5,5-tetramethyl-1,3,2- |
| | | dioxaborolan-2-yl)-benzenesulfonamide |
| 157 | B-2 | (3-chloro-4-fluoro-phenyl)boronic acid |
| 158 | B-2 | (2,3-dimethoxyphenyl)boronic acid |
| 159 | B-2 | [4-(tert-butoxycarbonylaminomethyl)phenyl]boronic acid |
| 160 | B-2 | (4-sulfamoylphenyl)boronic acid |
| 161 | B-2 | (3,4-dimethoxyphenyl)boronic acid |
| 162 | B-2 | [4-(methylsulfonylaminomethyl)phenyl]boronic acid |
| 163 | B-2 | 1-[4-(4,4,5,5-tetramethyl-1,3,2-dioxaborolan-2- |
| | | yl)phenyl]sulfonylpyrrolidin-3-ol |

Additional exemplary compounds 164-528, as shown in Table II.C-1, can also be prepared using appropriate starting materials and methods exemplified for the previously described compounds.

TABLE II.C-5
Physical data for exemplary compounds.

| Compound | LCMS | | |
|---|---|---|---|
| No. | [M + H]⁺ | LCMS RT | NMR |
| 1 | 416.3 | 2.39 | |
| 2 | 442.5 | 2.7 | |
| 3 | 427.1 | 4.1 | |
| 4 | 508.3 | 3.43 | |
| 5 | 423.3 | 3.72 | |
| 6 | 390.1 | 3.57 | |
| 7 | 402.5 | 2.96 | 1H NMR (400 MHz, CD3CN): d 1.21-1.29 (m, |
| | | | 2H), 1.62-1.68 (m, 2H), 3.05 (s, 6H), 6.06 (s, 2H), |
| | | | 6.86-6.97 (m, 3H), 7.04-7.08 (m, 2H), |
| | | | 7.53-7.55 (m, 1H), 7.76-7.82 (m, 3H), 7.86 (t, J = 8.0 Hz, |
| | | | 1H), 8.34 (br s, 1H) |
| 8 | 444.5 | 3.09 | |
| 9 | 430.5 | 2.84 | |
| 10 | 375.3 | 3.39 | |
| 11 | 403.5 | 2.83 | |
| 12 | 390 | 3.14 | |
| 14 | 520.18 | 1.38 | |
| 15 | 387.3 | 3.71 | |
| 16 | 389.3 | 2.9 | |
| 17 | 403.5 | 3.33 | |
| 18 | 403.5 | 3.75 | |
| 19 | 387.1 | 3.76 | |
| 20 | 389 | 2.79 | 1H NMR (400 MHz, CD3CN/DMSO-d6): d |
| | | | 1.15-1.23 (m, 2H), 1.56-1.61 (m, 2H), 4.60 (s, 2H), |
| | | | 6.05 (s, 2H), 6.94 (d, J = 8.3 Hz, 1H), |
| | | | 7.05-7.09 (m, 2H), 7.44 (d, J = 8.2 Hz, 2H), 7.57-7.62 (m, |
| | | | 2H), 7.92 (s, 1H), 8.00 (dd, J = 2.5, 8.6 Hz, 1H), |
| | | | 8.17 (d, J = 8.6 Hz, 1H), 8.48 (d, J = 1.8 Hz, 1H) |
| 21 | 360 | 2.18 | |
| 22 | 387.3 | 3.77 | |
| 23 | 535.17 | 2.81 | |
| 24 | 464.14 | 2.35 | 1H-NMR (DMSO d6, 300 MHz) d 8.40 (s, 1H), |
| | | | 7.96 (d, J = 8.4 Hz, 1H), 7.86 (m, 2H), 7.82 (m, |
| | | | 1H), 7.62 (d, J = 7.8 Hz, 1H), 7.36 (d, J = 7.8 Hz, |
| | | | 1H), 7.11 (d, J = 2.1 Hz, 1H), 7.00 (m, 2H), |
| | | | 6.05 (s, 2H), 3.42 (m, 2H, overlap with water), |
| | | | 3.03 (m, J = 5.4 Hz, 2H), 2.98 (t, 1H), 1.49 (m, 2H), |
| | | | 1.14 (m, 2H). |
| 25 | 403 | 3.29 | 1H NMR (400 MHz, CD3CN/DMSO-d6): d |
| | | | 1.14-1.17 (m, 2H), 1.52-1.55 (m, 2H), 6.01 (s, 2H), |
| | | | 6.03 (s, 2H), 6.89-6.96 (m, 2H), 7.01-7.12 (m, |
| | | | 3H), 7.15 (d, J = 1.8 Hz, 1H), 7.93 (dd, J = 8.7, |
| | | | 2.5 Hz, 1H), 8.05-8.11 (m, 2H), 8.39-8.41 (m, |
| | | | 1H) |
| 26 | 393 | 3.88 | |
| 27 | 452.1 | 3.11 | |
| 28 | 427.1 | 4.19 | |
| 29 | 388.9 | 3.58 | |
| 30 | 375.3 | 2.95 | |
| 31 | 535.1777 | 2.42 | |
| 32 | 359.1 | 3.48 | |
| 33 | 394.9 | 3.77 | |
| 34 | 360.3 | 2.96 | |
| 35 | 495.1464 | 2.24 | 1H-NMR (300 MHz, CDCl3) d 8.22 (d, J = 8.7 Hz, |
| | | | 1H), 7.98 (m, 3H), 7.80 (m, 3H), 7.45 (d, J = 7.5 Hz, |
| | | | 1H), 6.99 (dd, J = 8.1, 1.8 Hz, 2H), |
| | | | 6.95 (d, J = 1.5 Hz, 1H), 6.86 (d, J = 8.1 Hz, 1H), |
| | | | 6.02 (s, 2H), 3.77 (t, J = 5.1 Hz, 2H), 3.17 (m, J = 5.1 Hz, |
| | | | 2H), 2.85 (s, 3H), 1.70 (q, J = 3.6 Hz, 2H), |
| | | | 1.19 (q, J = 3.6 Hz, 2H). |
| 36 | 521.16 | 2.36 | 1H-NMR (300 MHz, DMSO-d6) 8.51 (s, 1H), |
| | | | 8.15 (d, J = 9.0 Hz, 2H), 8.06 (d, J = 8.4 Hz, 1H), |
| | | | 7.92 (t, J = 7.8 Hz, 1H), 7.88 (d, J = 8.1 Hz, 2H), |
| | | | 7.76 (d, J = 7.5 Hz, 1H), 7.11 (d, J = 1.2 Hz, 1H), |
| | | | 7.03 (dd, J = 7.8, 1.8 Hz, 1H), 6.97 (d, J = 7.8 Hz, |
| | | | 1H), 6.06 (s, 2H), 3.55 (m, 2H, overlap with |
| | | | water), 3.15 (m, 2H), 3.07 (m, 1H), 1.77 (m, 2H), |
| | | | 1.50 (dd, J = 7.2, 4.5 Hz, 2H), 1.43 (m, 2H), |
| | | | 1.15 (dd, J = 6.9, 3.9 Hz, 2H). |
| 37 | 452.3 | 3.38 | |
| 38 | 398 | 3.02 | |
| 39 | 483.12 | 2.58 | 1H-NMR (DMSO d6, 300 MHz) d 10.01 (t, J = 6.0 Hz, |
| | | | 1H), 8.39 (s, 1H), 7.97 (d, J = 7.8 Hz, 1H), |
| | | | 7.89 (d, J = 8.4 Hz, 1H), 7.83 (d, J = 7.8 Hz, 1H), |
| | | | 7.62 (d, J = 6.9 Hz, 1H), 7.33 (d, J = 8.4 Hz, 2H), |
| | | | 7.11 (d, J = 2.1 Hz, 1H), 7.03 (d, J = 1.5 Hz, 1H), |
| | | | 6.99 (dd, 7.8 Hz, 2H), 6.05 (s, 2H), 4.41 (d, J = 6 Hz, |
| | | | 2H), 1.48 (m, 2H), 1.14 (m, 2H). |
| 40 | 393.1 | 3.89 | |
| 41 | 373.1 | 3.57 | |
| 42 | 421.1 | 3.33 | |
| 43 | 417.3 | 3.62 | |
| 44 | 401.17 | 1.26 | |
| 45 | 403.5 | 3.25 | |
| 46 | 437.3 | 3.19 | |
| 47 | 391.1 | 3.82 | |
| 48 | 384.3 | 3.74 | |
| 49 | 419.3 | 3.27 | |
| 50 | 437 | 3.02 | |
| 51 | 349 | 3.33 | |
| 52 | 373.1 | 3.58 | 1H NMR (400 MHz, CD3CN): d 1.17-1.20 (m, |
| | | | 2H), 1.58-1.61 (m, 2H), 2.24 (s, 3H), 6.01 (s, 2H), |
| | | | 6.90 (d, J = 8.4 Hz, 1H), 7.04-7.06 (m, 2H), |
| | | | 7.16 (dd, J = 7.5, 0.8 Hz, 1H), 7.23-7.33 (m, 4H), |
| | | | 7.79-7.89 (m, 2H), 8.10 (dd, J = 8.3, 0.8 Hz, 1H) |
| 53 | 387 | 3.62 | |
| 54 | 394.1 | 3.06 | |
| 55 | 419.3 | 2.92 | |
| 56 | 407.5 | 3.55 | |
| 57 | 388.9 | 2.91 | |
| 58 | 360.2 | 3.74 | |
| 59 | 417.3 | 3.64 | |
| 60 | 402.5 | 3.07 | |
| 61 | 387.1 | 3.84 | |
| 62 | 415.3 | 4.1 | |
| 63 | 384 | 3.35 | |
| 64 | 360.3 | 3.58 | |
| 65 | 465.13 | 2.47 | 1H-NMR (300 MHz, CDCl3) 8.19 (d, J = 8.1 Hz, |
| | | | 1H), 7.97 (d, J = 8.4 Hz, 2H), 7.92 (s, 1H), |
| | | | 7.89 (d, J = 8.4 Hz, 2H), 7.76 (t, J = 7.5 Hz, 1H), |
| | | | 7.44 (d, J = 7.5 Hz, 1H), 6.99 (m, 1H), 6.95 (br s, 1H), |
| | | | 6.86 (d, J = 8.1 Hz, 1H), 6.02 (s, 2H), 4.37 (t, J = 5.7 Hz, |
| | | | 1H), 3.02 (m, 2H), 1.70 (q, J = 3.9 Hz, |
| | | | 2H), 1.17 (q, J = 3.6 Hz, 2H), 1.11 (t, J = 7.2 Hz, |
| | | | 3H). |
| 66 | 401 | 3.24 | |
| 67 | 393 | 3.88 | |
| 68 | 407.5 | 4.04 | |
| 69 | 377.1 | 3.26 | |
| 70 | 403.5 | 3.69 | |
| 71 | 472.3 | 3.02 | |
| 72 | 363 | 3.38 | |
| 73 | 449.3 | 3.4 | |
| 74 | 416.3 | 2.43 | |
| 75 | 373.1 | 3.69 | |
| 76 | 534.1936 | 1.36 | |
| 77 | 491.1514 | 2.7 | |
| 78 | 384.3 | 3.72 | |
| 79 | 388.3 | 2.32 | |
| 80 | 437.3 | 3.42 | |
| 81 | 373 | 3.51 | 1H NMR (400 MHz, CD3CN/DMSO-d6): d |
| | | | 1.07-1.27 (m, 2H), 1.50-1.67 (m, 2H), 2.36 (s, 3H), |
| | | | 6.10 (s, 2H), 6.92 (d, J = 7.9 Hz, 1H), |
| | | | 7.01-7.09 (m, 2H), 7.28 (d, J = 7.9 Hz, 2H), 7.50 (d, J = 8.2 Hz, |
| | | | 2H), 7.93-8.00 (m, 2H), 8.15 (d, J = 9.3 Hz, |
| | | | 1H), 8.44 (d, J = 2.5 Hz, 1H) |
| 82 | 419 | 2.71 | 1H NMR (400 MHz, CD3CN): d 1.29-1.32 (m, |
| | | | 2H), 1.68-1.71 (m, 2H), 3.90 (s, 3H), 3.99 (s, 3H), |
| | | | 6.04 (s, 2H), 6.70-6.72 (m, 2H), 6.93 (d, J = 8.4 Hz, |
| | | | 1H), 7.03-7.05 (m, 2H), 7.59 (d, J = 8.2 Hz, |
| | | | 1H), 7.73 (t, J = 7.6 Hz, 2H), 8.01 (t, J = 8.1 Hz, |
| | | | 1H), 8.72 (br s, 1H) |
| 83 | 417.3 | 3.41 | |
| 84 | 394.9 | 3.74 | |
| 85 | 401.3 | 3.97 | |
| 86 | 473.5 | 2.69 | |
| 87 | 419.1 | 3.18 | 1H NMR (400 MHz, CD3CN): d 1.25-1.31 (m, |
| | | | 2H), 1.62-1.69 (m, 2H), 3.84 (s, 3H), 3.86 (s, 3H), |
| | | | 6.04 (s, 2H), 6.62-6.70 (m, 2H), 6.92 (d, J = 8.4 Hz, |
| | | | 1H), 7.00-7.08 (m, 2H), 7.30 (d, J = 8.3 Hz, |
| | | | 1H), 7.96 (d, J = 8.9 Hz, 1H), 8.14 (dd, J = 8.9, |
| | | | 2.3 Hz, 1H), 8.38 (d, J = 2.2 Hz, 1H), 8.65 (br s, |
| | | | 1H) |
| 88 | 399 | 3.83 | |
| 89 | 401.3 | 3.62 | |
| 90 | 407.3 | 3.59 | |
| 91 | 505.17 | 2.88 | |
| 92 | 384 | 3.36 | 1H NMR (400 MHz, CD3CN): d 1.27-1.30 (m, |
| | | | 2H), 1.65-1.67 (m, 2H), 6.05 (s, 2H), 6.93 (d, J = 8.4 Hz, |
| | | | 1H), 7.04-7.09 (m, 2H), 7.67 (t, J = 7.7 Hz, |
| | | | 1H), 7.79-7.81 (m, 1H), 7.91-7.94 (m, 1H), |
| | | | 8.02-8.08 (m, 2H), 8.23 (dd, J = 8.9, 2.5 Hz, 1H), |
| | | | 8.50 (d, J = 1.9 Hz, 1H), 8.58 (br s, 1H) |
| 93 | 402 | 2.73 | 1H NMR (400 MHz, CD3CN): d 1.16-1.24 (m, |
| | | | 2H), 1.57-1.62 (m, 2H), 6.05 (s, 2H), 6.95 (d, J = 7.6 Hz, |
| | | | 1H), 7.05-7.09 (m, 2H), 7.71-7.75 (m, |
| | | | 2H), 7.95 (br s, 1H), 8.04-8.10 (m, 3H), 8.22 (d, J = 8.7 Hz, |
| | | | 1H), 8.54 (d, J = 2.5 Hz, 1H) |
| 94 | 419.3 | 2.8 | |
| 95 | 403.3 | 2.98 | |
| 97 | 416.5 | 3.22 | |
| 98 | 421 | 3 | |
| 99 | 407.1 | 3.32 | |
| 100 | 389 | 2.83 | 1H NMR (400 MHz, CD3CN): d 1.21-1.26 (m, |
| | | | 2H), 1.60-1.65 (m, 2H), 4.65 (s, 2H), 6.03 (s, 2H), |
| | | | 6.89-6.94 (m, 1H), 7.02-7.08 (m, 2H), |
| | | | 7.36-7.62 (m, 3H), 8.12 (s, 2H), 8.36 (br s, 1H), |
| | | | 8.45-8.47 (m, 1H) |
| 101 | 388.9 | 3.27 | 1H NMR (400 MHz, CD3CN): d 1.22-1.24 (m, |
| | | | 2H), 1.61-1.63 (m, 2H), 3.82 (s, 3H), 6.04 (s, 2H), |
| | | | 6.92 (d, J = 8.4 Hz, 1H), 7.04-7.12 (m, 4H), |
| | | | 7.34 (dd, J = 7.6, 1.7 Hz, 1H), 7.38-7.43 (m, 1H), |
| | | | 8.03 (dd, J = 8.7, 2.3 Hz, 1H), 8.10 (dd, J = 8.7, 0.7 Hz, |
| | | | 1H), 8.27 (br s, 1H), 8.37-8.39 (m, 1H) |
| 102 | 401.3 | 3.77 | |
| 103 | 430.5 | 3.04 | |
| 104 | 388.3 | 2.32 | |
| 105 | 521.162 | 2.46 | |
| 106 | 393 | 3.63 | |
| 107 | 416 | 2.84 | 1H NMR (400 MHz, CD3CN/DMSO-d6): d |
| | | | 1.13-1.22 (m, 2H), 1.53-1.64 (m, 2H), 2.07 (s, 3H), |
| | | | 6.08 (s, 2H), 6.90-6.95 (m, 1H), 7.01-7.09 (m, |
| | | | 2H), 7.28 (d, J = 8.8 Hz, 1H), 7.37 (t, J = 7.9 Hz, |
| | | | 1H), 7.61 (d, J = 8.8 Hz, 1H), 7.84 (d, J = 1.6 Hz, |
| | | | 1H), 7.95 (dd, J = 2.5, 8.7 Hz, 1H), 8.03 (br s, |
| | | | 1H), 8.16 (d, J = 8.7 Hz, 1H), 8.42 (d, J = 2.4 Hz, |
| | | | 1H), 9.64 (s, 1H) |
| 108 | 403.3 | 3.07 | |
| 109 | 349.1 | 3.29 | |
| 110 | 389.2 | 3.15 | |
| 111 | 521.162 | 2.27 | |
| 112 | 394 | 3.82 | |
| 113 | 407.5 | 3.3 | |
| 114 | 417.1 | 3.17 | |
| 115 | 398.1 | 3.22 | |
| 116 | 394 | 3.1 | 1H NMR (400 MHz, CD3CN): d 1.18-1.26 (m, |
| | | | 2H), 1.59-1.64 (m, 2H), 6.05 (s, 2H), 6.95 (d, J = 8.4 Hz, |
| | | | 1H), 7.06-7.11 (m, 2H), 7.40 (d, J = 4.9 Hz, |
| | | | 1H), 7.92-7.96 (m, 2H), 8.26 (d, J = 9.3 Hz, |
| | | | 1H), 8.36 (d, J = 1.7 Hz, 1H), 8.56 (d, J = 5.0 Hz, |
| | | | 1H), 8.70 (s, 1H) |
| 117 | 363.3 | 3.48 | |
| 118 | 374.3 | 3.54 | |
| 119 | 494.3 | 3.59 | |
| 120 | 505.2 | 2.9 | |
| 121 | 374.3 | 2.55 | |
| 122 | 417.3 | 3.63 | |
| 123 | 389.3 | 3.47 | |
| 124 | 417.1 | 3.29 | |
| 125 | 417.3 | 3.08 | |
| 126 | 427.3 | 3.89 | |
| 127 | 535.2 | 2.76 | |
| 128 | 386.9 | 3.67 | |
| 129 | 377.1 | 3.67 | |
| 130 | 389.1 | 3.4 | 1H NMR (400 MHz, CD3CN): d 1.22-1.24 (m, |
| | | | 2H), 1.61-1.63 (m, 2H), 3.86 (s, 3H), 6.05 (s, 2H), |
| | | | 6.93 (d, J = 8.4 Hz, 1H), 6.97-7.00 (m, 1H), |
| | | | 7.05-7.08 (m, 2H), 7.16-7.21 (m, 2H), 7.41 (t, J = 8.0 Hz, |
| | | | 1H), 8.07-8.17 (m, 3H), 8.48-8.48 (m, 1H) |
| 131 | 407.3 | 3.49 | |
| 132 | 419 | 3.09 | 1H NMR (400 MHz, CD3CN): d 1.17-1.25 (m, |
| | | | 2H), 1.57-1.64 (m, 2H), 3.72 (s, 6H), 6.04 (s, 2H), |
| | | | 6.74 (d, J = 8.4 Hz, 2H), 6.93 (d, J = 8.4 Hz, 1H), |
| | | | 7.05-7.08 (m, 2H), 7.35 (t, J = 8.4 Hz, 1H), |
| | | | 7.75 (d, J = 10.5 Hz, 1H), 8.07-8.14 (m, 3H) |
| 133 | 431.3 | 3.27 | |
| 135 | 417.3 | 3.81 | |
| 136 | 535.2 | 2.75 | |
| 137 | 403.5 | 3.35 | |
| 138 | 432.5 | 2.76 | H NMR (400 MHz, CD3CN) 1.30-1.35 (m, 2H), |
| | | | 1.69-1.74 (m, 2H), 3.09 (s, 6H), 4.05 (s, 3H), |
| | | | 6.04 (s, 2H), 6.38 (d, J = 2.4 Hz, 1H), 6.50 (dd, J = 9.0, |
| | | | 2.4 Hz, 1H), 6.93 (d, J = 8.4 Hz, 1H), |
| | | | 7.03-7.06 (m, 2H), 7.31 (d, J = 7.7 Hz, 1H), 7.71 (d, J = 8.8 Hz, |
| | | | 2H), 7.97 (t, J = 8.3 Hz, 1H) |
| 139 | 421.1 | 2.71 | |
| 140 | 416.5 | 2.92 | |
| 141 | 410 | 2.83 | 1H NMR (400 MHz, CD3CN): d 1.28-1.37 (m, |
| | | | 2H), 1.66-1.73 (m, 2H), 6.05 (s, 2H), |
| | | | 6.91-6.97 (m, 1H), 7.05-7.09 (m, 2H), 7.69-7.74 (m, 1H), |
| | | | 7.82 (t, J = 7.7 Hz, 1H), 7.93 (d, J = 7.2 Hz, 1H), |
| | | | 8.04 (d, J = 8.8 Hz, 1H), 8.15 (d, J = 8.2 Hz, 1H), |
| | | | 8.37 (d, J = 8.8 Hz, 1H), 8.58-8.65 (m, 2H), |
| | | | 8.82 (br s, 1H), 8.94 (d, J = 6.2 Hz, 1H) |
| 142 | 349.3 | 3.33 | |
| 143 | 373.1 | 3.68 | |
| 144 | 535.1777 | 2.33 | |
| 145 | 390.3 | 3.4 | |
| 146 | 386.9 | 3.72 | |
| 147 | 419.1 | 3.13 | 1H NMR (400 MHz, CD3CN): d 1.23-1.26 (m, |
| | | | 2H), 1.62-1.64 (m, 2H), 3.86 (s, 3H), 3.89 (s, 3H), |
| | | | 6.04 (s, 2H), 6.93 (d, J = 8.4 Hz, 1H), |
| | | | 7.03-7.07 (m, 3H), 7.17-7.19 (m, 2H), 8.06-8.15 (m, 2H), |
| | | | 8.38 (br s, 1H), 8.45-8.46 (m, 1H) |
| 148 | 393.1 | 3.72 | 1H NMR (400 MHz, CD3CN): d 1.20-1.27 (m, |
| | | | 2H), 1.58-1.67 (m, 2H), 6.05 (s, 2H), 6.94 (d, J = 8.4 Hz, |
| | | | 1H), 7.05-7.09 (m, 2H), 7.41-7.50 (m, |
| | | | 2H), 7.55-7.59 (m, 1H), 7.66-7.69 (m, 1H), |
| | | | 8.07 (d, J = 11.2 Hz, 1H), 8.11 (br s, 1H), 8.16 (d, J = 8.8 Hz, |
| | | | 1H), 8.48 (d, J = 1.9 Hz, 1H) |
| 149 | 458.5 | 2.42 | |
| 150 | 403.5 | 3.04 | |
| 151 | 452.3 | 3.44 | H NMR (400 MHz, MeOD) 1.30-1.36 (m, 2H), |
| | | | 1.71-1.77 (m, 2H), 2.58 (s, 3H), 6.04 (s, 2H), |
| | | | 6.93 (dd, J = 0.8, 7.5 Hz, 1H), 7.04-7.08 (m, 2H), |
| | | | 7.86 (dd, J = 0.8, 7.7 Hz, 1H), 8.00-8.02 (m, 2H), |
| | | | 8.08-8.12 (m, 3H), 8.19-8.23 (m, 1H) |
| 152 | 403 | 2.97 | |
| 153 | 359.1 | 3.36 | 1H NMR (400 MHz, CD3CN): d 1.24-1.26 (m, |
| | | | 2H), 1.62-1.65 (m, 2H), 6.05 (s, 2H), 6.93 (d, J = 8.4 Hz, |
| | | | 1H), 7.05-7.08 (m, 2H), 7.42-7.46 (m, |
| | | | 1H), 7.49-7.53 (m, 2H), 7.63-7.66 (m, 2H), |
| | | | 8.10-8.16 (m, 2H), 8.33 (br s, 1H), 8.48-8.48 (m, 1H) |
| 154 | 395.1 | 3.34 | |
| 155 | 393 | 3.7 | |
| 156 | 390.2 | 3.7 | |
| 157 | 403.5 | 3.33 | |
| 158 | 390.2 | 3.58 | |
| 159 | 493.1671 | 2.85 | |
| 160 | 411.3 | 3.94 | |
| 161 | 419.1 | 3.2 | |
| 162 | 488.1 | 3.62 | |
| 163 | 438.1 | 3 | |
| 164 | 314.1419 | 3.38 | |
| 165 | 538.5 | 3.28 | |
| 166 | 466.1 | 2.9 | |
| 167 | 429.3 | 2.95 | |
| 168 | 526.3422 | 3.189189 | |
| 169 | 498.3 | 3.7 | |
| 170 | 468.3 | 3.27 | |
| 171 | 444.5 | 2.24 | |
| 172 | 551.1496 | 2.849824 | |
| 173 | 377 | 3.7 | |
| 174 | 493.9 | 2.69 | |
| 175 | 517.9397 | 3.423179 | |
| 176 | 522.341 | 3.49262 | |
| 177 | 502.1 | 3.43 | |
| 178 | 549.149 | 2.906129 | |
| 179 | 480.1 | 2.51 | |
| 180 | 520.3405 | 4.295395 | |
| 181 | 488.2 | 3.07 | |
| 182 | 535.1448 | 3.267469 | |
| 183 | 436.3 | 3.62 | |
| 184 | 496.3333 | 3.265482 | |
| 185 | 403.5 | 2.88 | |
| 186 | 420.9 | 2.86 | |
| 187 | 444.3 | 2.39 | |
| 188 | 417.3 | 2.24 | |
| 189 | 466.1 | 2.88 | |
| 190 | 438.1 | 2.39 | |
| 191 | 401.1 | 3.44 | |
| 192 | 552.3 | 3.18 | |
| 193 | 452.3 | 2.55 | |
| 194 | 415 | 4 | |
| 195 | 479.1 | 1.08 | |
| 196 | 430.5 | 2.34 | |
| 197 | 512.3381 | 2.961206 | |
| 198 | 444.5 | 2.75 | H NMR (400 MHz, DMSO-d6) 1.11-1.19 (m, |
| | | | 2H), 1.46-1.52 (m, 2H), 2.31 (s, 3H), 2.94 (s, 3H), |
| | | | 2.99 (s, 3H), 6.08 (s, 2H), 6.97-7.05 (m, 2H), |
| | | | 7.13 (d, J = 1.6 Hz, 1H), 7.35 (t, J = 1.5 Hz, 1H), |
| | | | 7.41 (t, J = 7.8 Hz, 2H), 7.51 (t, J = 7.6 Hz, 1H), |
| | | | 7.68 (d, J = 8.4 Hz, 1H), 7.97 (d, J = 8.4 Hz, 1H), |
| | | | 8.34 (s, 1H) |
| 199 | 540.3464 | 3.182981 | |
| 200 | 520.3 | 3.79 | |
| 201 | 452.3 | 3.22 | |
| 202 | 536.5 | 3.63 | |
| 203 | 509.1371 | 2.815619 | |
| 204 | 444.5 | 2.5 | |
| 205 | 524.3416 | 3.476111 | |
| 206 | 407.5 | 3.6 | |
| 207 | 452.1 | 2.62 | |
| 208 | 520.3405 | 4.058878 | |
| 209 | 416.1 | 2.3 | |
| 210 | 452.3 | 2.8 | H NMR (400 MHz, DMSO-d6) 1.11-1.19 (m, |
| | | | 2H), 1.47-1.52 (m, 2H), 2.31 (s, 6.08 (s, 2H), |
| | | | 6.96-7.07 (m, 2H), 7.13 (d, J = 1.6 Hz, 1H), |
| | | | 7.43 (s, 1H), 7.57 (d, J = 8.1 Hz, 2H), 7.69 (d, J = 8.5 Hz, |
| | | | 2H), 7.89 (d, J = 8.2 Hz, 2H), 7.99 (d, J = 8.4 Hz, |
| | | | 1H), 8.38 (s, 1H) |
| 211 | 480.3 | 3.33 | |
| 212 | 521.1407 | 3.231696 | |
| 213 | 415.3 | 3.4 | |
| 214 | 562.3 | 3.71 | |
| 215 | 403.3 | 2.67 | |
| 216 | 421.1 | 2.91 | |
| 217 | 387.1 | 2.89 | |
| 218 | 488.3 | 3.73 | |
| 219 | 403.7 | 2.43 | |
| 220 | 508.5 | 3.46 | |
| 221 | 508.3 | 3.46 | |
| 222 | 401.1 | 2.76 | |
| 223 | 484.5 | 3.95 | |
| 224 | 407.5 | 3.23 | |
| 225 | 401.2 | 3.49 | |
| 226 | 608.3 | 3.58 | |
| 227 | 417.1 | 2.24 | |
| 228 | 452.3 | 3.21 | |
| 229 | 407.1 | 3.08 | |
| 230 | 401.3 | 2.68 | |
| 231 | 389.1 | 2.36 | |
| 232 | 481.9291 | 3.155919 | |
| 233 | 535.9451 | 3.577682 | |
| 234 | 551.1496 | 2.903536 | |
| 235 | 415.3 | 3.71 | H NMR (400 MHz, DMSO-d6) 1.12-1.17 (m, |
| | | | 2H), 1.23 (d, J = 6.9 Hz, 6H), 1.47-1.51 (m, 2H), |
| | | | 2.30 (s, 3H), 2.92 (septet, J = 6.9 Hz, 1H), 6.08 (s, |
| | | | 2H), 6.97-7.05 (m, 2H), 7.12-7.17 (m, 2H), |
| | | | 7.20-7.22 (m, 1H), 7.24-7.26 (m, 1H), 7.36 (t, J = 7.6 Hz, |
| | | | 1H), 7.65 (d, J = 8.4 Hz, 1H), 7.95 (d, J = 8.4 Hz, |
| | | | 1H), 8.32 (s, 1H) |
| 236 | 540.3 | 3.85 | |
| 237 | 456.5 | 3.35 | |
| 238 | 416.5 | 2.35 | |
| 239 | 529.3 | 2.29 | |
| 240 | 442.3 | 3.57 | |
| 241 | 466.3 | 3.5 | |
| 242 | 506.3 | 3.67 | |
| 243 | 403.3 | 2.69 | |
| 244 | 534.3446 | 3.933966 | |
| 245 | 466.3 | 3.6 | |
| 246 | 496.3 | 2.9 | |
| 247 | 458.5 | 2.3 | |
| 248 | 450.3 | 3.01 | |
| 249 | 565.1537 | 2.890517 | |
| 250 | 480.5 | 3.74 | |
| 251 | 452.1 | 1.07 | |
| 252 | 389.1 | 2.82 | |
| 253 | 530.3 | 2.8 | |
| 254 | 466.1 | 1.06 | |
| 255 | 488.2 | 3.05 | |
| 256 | 558.3 | 3.46 | |
| 257 | 407.5 | 3.27 | |
| 258 | 430.5 | 2.66 | H NMR (400 MHz, DMSO-d6) 1.12-1.18 (m, |
| | | | 2H), 1.47-1.54 (m, 2H), 2.30 (s, 3H), 2.79 (d, J = 4.5 Hz, |
| | | | 3H), 6.08 (s, 2H), 6.96-7.07 (m, 2H), |
| | | | 7.13 (d, J = 1.6 Hz, 1H), 7.48-7.57 (m, 2H), 7.70 (d, J = 8.4 Hz, |
| | | | 1H), 7.78 (d, J = 1.5 Hz, 1H), 7.84 (dt, J = 7.3, |
| | | | 1.7 Hz, 1H), 7.98 (d, J = 8.4 Hz, 1H), |
| | | | 8.36 (s, 1H), 8.50-8.51 (m, 1H) |
| 259 | 470.3 | 3.82 | |
| 260 | 403.1 | 2.27 | |
| 261 | 549.149 | 3.390635 | |
| 262 | 438.1 | 3.43 | |
| 263 | 403.3 | 2.8 | |
| 264 | 407.1 | 3.04 | |
| 265 | 430.5 | 2.18 | |
| 266 | 403.3 | 2.96 | |
| 267 | 531.9439 | 2.812401 | |
| 268 | 496.3333 | 3.24369 | |
| 269 | 373.5 | 2.76 | |
| 270 | 520.3405 | 4.209111 | |
| 271 | 450.3 | 3.77 | |
| 272 | 403.2 | 1.09 | |
| 273 | 543.1472 | 2.891489 | |
| 274 | 417.3 | 2.26 | |
| 275 | 527.9427 | 3.907424 | |
| 276 | 510.3375 | 3.374722 | |
| 277 | 403.1 | 2.2 | |
| 278 | 430.5 | 2.68 | H NMR (400 MHz, DMSO-d6) 1.12-1.19 (m, |
| | | | 2H), 1.47-1.51 (m, 2H), 2.31 (s, 3H), 2.80 (d, J = 4.5 Hz, |
| | | | 3H), 6.08 (s, 2H), 6.97-7.05 (m, 2H), |
| | | | 7.13 (d, J = 1.6 Hz, 1H), 7.45 (d, J = 8.4 Hz, 2H), |
| | | | 7.68 (d, J = 8.4 Hz, 1H), 7.90 (d, J = 8.5 Hz, 2H), |
| | | | 7.97 (d, J = 8.3 Hz, 1H), 8.35 (s, 1H), 8.50 (q, J = 4.5 Hz, |
| | | | 1H) |
| 279 | 536.5 | 3.19 | |
| 280 | 480.3 | 3.25 | |
| 281 | 550.5 | 3.78 | |
| 282 | 482.5 | 3.15 | |
| 283 | 416.3 | 2.58 | |
| 284 | 554.3 | 3.99 | |
| 285 | 546.3481 | 2.872586 | |
| 286 | 416.1 | 2.29 | |
| 287 | 443 | 4.02 | |
| 288 | 466.3 | 2.76 | |
| 289 | 373.1 | 2.84 | |
| 290 | 429.3 | 3 | |
| 291 | 403.1 | 2.24 | |
| 292 | 479.15 | 2.49 | |
| 293 | 417.3 | 2.65 | |
| 294 | 403.5 | 2.39 | |
| 295 | 416.3 | 2.61 | H NMR (400 MHz, DMSO-d6) 1.14-1.18 (m, |
| | | | 2H), 1.46-1.54 (m, 2H), 2.31 (s, 3H), 6.08 (s, 2H), |
| | | | 6.97-7.05 (m, 2H), 7.13 (d, J = 1.6 Hz, 1H), |
| | | | 7.44 (s, 1H), 7.49-7.56 (m, 2H), 7.72 (d, J = 8.4 Hz, |
| | | | 1H), 7.83-7.85 (m, 1H), 7.87-7.91 (m, 1H), |
| | | | 7.99 (d, J = 8.4 Hz, 1H), 8.05 (s, 1H), 8.39 (s, 1H) |
| 296 | 387.1 | 3.09 | |
| 297 | 430.2 | 2.38 | |
| 298 | 403.2 | 2.72 | |
| 299 | 387.3 | 2.86 | |
| 300 | 387.3 | 3.03 | |
| 301 | 403.5 | 2.44 | |
| 302 | 508.3 | 3.45 | |
| 303 | 417.3 | 2.58 | |
| 304 | 549.149 | 3.346045 | |
| 305 | 429.5 | 3.01 | |
| 306 | 492.3321 | 3.811817 | |
| 307 | 512.3381 | 2.973403 | |
| 308 | 415.3 | 2.85 | |
| 309 | 444.5 | 2.75 | |
| 310 | 430.5 | 2.41 | |
| 311 | 534.3446 | 3.920694 | |
| 312 | 492.3321 | 3.992977 | |
| 313 | 387.3 | 2.84 | |
| 314 | 430.5 | 2.37 | |
| 315 | 387 | 1.12 | |
| 316 | 526.3422 | 3.08259 | |
| 317 | 344.1524 | 3.35 | |
| 318 | 536.5 | 3.17 | |
| 319 | 492.3 | 3.69 | |
| 320 | 430.2 | 2.38 | |
| 321 | 452.3 | 2.55 | |
| 322 | 387.1 | 2.6 | |
| 323 | 387.1 | 3.01 | |
| 324 | 402.5 | 2.14 | |
| 325 | 531.9439 | 3.830608 | |
| 326 | 444.5 | 2.5 | |
| 327 | 403.3 | 2.83 | |
| 328 | 401.1 | 3.48 | |
| 329 | 415.3 | 3.36 | |
| 330 | 522.341 | 4.140655 | |
| 331 | 387.1 | 3.01 | |
| 332 | 505.9362 | 4.059895 | |
| 333 | 417.1 | 2.58 | |
| 334 | 403.5 | 2.92 | |
| 335 | 520.3405 | 4.215356 | |
| 336 | 510.3375 | 3.363424 | |
| 337 | 401.1 | 2.73 | |
| 338 | 479.9284 | 3.436073 | |
| 339 | 508.3369 | 3.825972 | |
| 340 | 512.5 | 3.6 | |
| 341 | 452.3 | 3.15 | |
| 342 | 540.3464 | 3.06556 | |
| 343 | 480.3 | 3 | |
| 344 | 526.3422 | 3.151655 | |
| 345 | 422.1 | 3.21 | |
| 346 | 415 | 4.05 | |
| 347 | 523.1413 | 3.095885 | |
| 348 | 416.3 | 1.87 | |
| 349 | 438.1 | 2.4 | |
| 350 | 402.5 | 2.18 | |
| 351 | 373.1 | 3.08 | |
| 352 | 415.7 | 3.13 | |
| 353 | 420.9 | 2.9 | |
| 354 | 407.3 | 3.03 | |
| 355 | 480.3 | 2.96 | |
| 356 | 452.3 | 2.47 | |
| 357 | 466.3 | 2.63 | |
| 358 | 536.5 | 3.26 | |
| 359 | 402.1 | 2.2 | |
| 360 | 510.3375 | 3.420695 | |
| 361 | 407 | 3.11 | |
| 362 | 494.5 | 3.45 | |
| 363 | 438.1 | 3.42 | |
| 364 | 535.9451 | 3.443787 | |
| 365 | 402.1 | 2.21 | |
| 366 | 565.1538 | 3.006094 | |
| 367 | 403.5 | 2.36 | |
| 368 | 444.5 | 2.97 | |
| 369 | 408.5 | 3.43 | |
| 370 | 403.3 | 2.45 | |
| 371 | 430.5 | 2.43 | |
| 372 | 478.3 | 3.47 | |
| 373 | 524.3416 | 3.499365 | |
| 374 | 466.3 | 2.35 | |
| 375 | 416.5 | 2.36 | |
| 376 | 552.3 | 3.42 | |
| 377 | 524.5 | 3.17 | |
| 378 | 538.5 | 3.07 | |
| 379 | 528.3 | 3.33 | |
| 380 | 548.3 | 3.75 | |
| 381 | 526.3 | 3.46 | |
| 382 | 520.5 | 3.48 | |
| 383 | 518.1 | 3.55 | |
| 384 | 542.3 | 3.59 | |
| 385 | 550.5 | 3.69 | |
| 386 | 524.3 | 3.15 | |
| 387 | 522.5 | 3.78 | |
| 388 | 542.2 | 3.6 | |
| 389 | 467.3 | 1.93 | |
| 390 | 469.3 | 1.99 | |
| 391 | 507.5 | 2.12 | |
| 392 | 453.5 | 1.99 | |
| 393 | 487.3 | 2.03 | |
| 394 | 483.5 | 1.92 | |
| 395 | 441.3 | 4.33 | |
| 396 | 453.3 | 1.93 | H NMR (400 MHz, DMSO-d6) 9.14 (s, 1H), |
| | | | 7.99-7.93 (m, 3H), 7.80-7.78 (m, 1H), |
| | | | 7.74-7.72 (m, 1H), 7.60-7.55 (m, 2H), 7.41-7.33 (m, 2H), |
| | | | 2.24 (s, 3H), 1.53-1.51 (m, 2H), 1.19-1.17 (m, |
| | | | 2H) |
| 397 | 439.5 | 1.94 | |
| 398 | 471.3 | 2 | |
| 399 | 537.5 | 2.1 | |
| 400 | 525.3 | 2.19 | |
| 401 | 453.5 | 1.96 | |
| 402 | 483.3 | 1.87 | |
| 403 | 457.5 | 1.99 | |
| 404 | 469.5 | 1.95 | |
| 405 | 471.3 | 1.98 | |
| 406 | 525.3 | 2.15 | |
| 407 | 439.4 | 1.97 | |
| 408 | 525.1 | 2.14 | |
| 409 | 618.7 | 3.99 | |
| 410 | 374.5 | 2.46 | |
| 411 | 507.5 | 2.14 | |
| 412 | 390.1 | 3.09 | |
| 413 | 552.3 | 4.04 | |
| 414 | 457.5 | 2.06 | |
| 415 | 521.5 | 2.14 | |
| 416 | 319 | 3.32 | |
| 417 | 471.3 | 1.96 | |
| 418 | 417.3 | 1.75 | |
| 419 | 473.3 | 2.04 | |
| 420 | 389.3 | 2.94 | |
| 421 | 457.5 | 1.99 | |
| 422 | 467.3 | 1.96 | |
| 423 | 430.7 | 1.54 | |
| 424 | 448.1 | 1.74 | |
| 425 | 594.5 | 1.99 | |
| 426 | 466.5 | 1.93 | |
| 427 | 467.3 | 1.89 | |
| 428 | 393.3 | 2.09 | |
| 429 | 494.5 | 1.34 | |
| 430 | 452.3 | 1.75 | |
| 431 | 416.5 | 1.48 | |
| 432 | 429.3 | 2.41 | |
| 433 | 449.3 | 1.73 | |
| 434 | 481.3 | 1.89 | |
| 435 | 515.5 | 1.81 | |
| 436 | 507.3 | 2.02 | |
| 437 | 425.3 | 1.64 | |
| 438 | 575.3 | 2.13 | |
| 439 | 409.3 | 2.24 | |
| 440 | 539.5 | 2.2 | |
| 441 | 409.1 | 2.11 | |
| 442 | 488.3 | 1.81 | |
| 443 | 507.3 | 2 | |
| 444 | 495.5 | 1.63 | |
| 445 | 389.5 | 1.43 | |
| 446 | 373.3 | 1.81 | |
| 447 | 393.3 | 2.11 | |
| 448 | 465.3 | 1.96 | H NMR (400 MHz, DMSO) 8.99 (s, 1H), |
| | | | 7.94-7.86 (m, 3H), 7.76-7.73 (m, 2H), 7.56 (d, J = 1.5 Hz, |
| | | | 1H), 7.41-7.33 (m, 2H), 5.47 (s, 2H), |
| | | | 2.26 (s, 3H), 1.53-1.50 (m, 2H), 1.19-1.16 (m, 2H) |
| 449 | 469.3 | 1.67 | H NMR (400 MHz, DMSO) 9.10 (s, 1H), 8.06 (d, |
| | | | J = 1.5 Hz, 1H), 8.01-7.93 (m, 3H), 7.76 (d, J = 7.5 Hz, |
| | | | 1H), 7.57-7.54 (m, 2H), 7.40-7.34 (m, |
| | | | 2H), 5.33 (s, 1H), 4.38 (s, 2H), 1.53-1.51 (m, |
| | | | 2H), 1.19-1.16 (m, 2H) |
| 450 | 430.7 | 1.64 | |
| 451 | 425.3 | 1.72 | |
| 452 | 389.5 | 1.68 | |
| 453 | 499.5 | 1.56 | |
| 454 | 438.7 | 1.66 | |
| 455 | 416.5 | 1.47 | |
| 456 | 453.3 | 2.03 | |
| 457 | 472.5 | 1.64 | |
| 458 | 427.5 | 1.45 | |
| 459 | 438.5 | 4.51 | |
| 460 | 495.5 | 1.63 | |
| 461 | 478.3 | 2.33 | |
| 462 | 426.3 | 1.49 | |
| 463 | 359.3 | 1.9 | |
| 465 | 499.5 | 1.61 | |
| 466 | 488.3 | 1.83 | |
| 467 | 469.3 | 1.91 | |
| 468 | 389.5 | 1.8 | |
| 469 | 464 | 1.39 | |
| 470 | 373.3 | 1.84 | |
| 471 | 467.3 | 1.96 | |
| 472 | 467.3 | 1.9 | |
| 473 | 388.5 | 1.23 | |
| 474 | 425 | 1.32 | |
| 475 | 483.5 | 1.86 | |
| 476 | 412.5 | 1.29 | |
| 477 | 497.3 | 1.93 | |
| 478 | 452.3 | 1.66 | |
| 479 | 478.1 | 2.34 | |
| 480 | 530.2 | 1.79 | 1H NMR (400 MHz, CD3CN) 9.57 (s, 1H) |
| | | | 8.01 (d, J = 8.4 Hz, 1H), 7.91-7.87 (m, 1H), 7.75 (s, |
| | | | 1H), 7.68-7.66 (m, 2H), 7.58-7.53 (m, 1H), |
| | | | 7.36-7.32 (m, 2H), 7.21 (d, J = 8.2 Hz, 1H), 3.30 (s, |
| | | | 3H), 2.25 (s, 3H), 1.63-1.58 (m, 2H), |
| | | | 1.20-1.16 (m, 2H). |
| 481 | 389.5 | 1.41 | |
| 482 | 473.1 | 2.06 | |
| 483 | 480.3 | 1.66 | |
| 484 | 388.5 | 1.27 | |
| 485 | 393.3 | 2.13 | |
| 486 | 469.3 | 1.67 | |
| 487 | 486.5 | 2.02 | |
| 488 | 388.5 | 1.32 | |
| 489 | 458.7 | 1.83 | |
| 490 | 467.3 | 1.94 | |
| 491 | 453.3 | 2.04 | |
| 492 | 402.5 | 1.44 | |
| 493 | 482.9 | 1.61 | |
| 494 | 469.3 | 1.92 | |
| 495 | 464.3 | 1.66 | |
| 496 | 516.5 | 1.96 | |
| 497 | 389.5 | 1.68 | |
| 498 | 441 | 1.89 | |
| 499 | 459 | 2.16 | |
| 500 | 454.5 | 1.81 | H NMR (400 MHz, DMSO) 9.59 (s, 1H), 9.08 (s, |
| | | | 1H), 8.10 (d, J = 1.6 Hz, 1H), 8.02 (d, J = 7.8 Hz, |
| | | | 1H), 7.85 (d, J = 7.7 Hz, 1H), 7.62 (t, J = 7.7 Hz, |
| | | | 1H), 7.54 (d, J = 1.6 Hz, 1H), 7.38 (d, J = 8.3 Hz, |
| | | | 1H), 7.32 (dd, J = 1.7, 8.3 Hz, 1H), 2.54 (s, 3H), |
| | | | 1.56-1.54 (m, 2H), 1.22-1.19 (m, 2H) |
| 501 | 492.3 | 1.75 | H NMR (400 MHz, DMSO) 8.78 (s, 1H), 8.12 (s, |
| | | | 1H), 7.88 (d, J = 8.4 Hz, 1H), 7.72 (d, J = 8.5 Hz, |
| | | | 1H), 7.57 (d, J = 1.6 Hz, 1H), 7.44-7.34 (m, 6H), |
| | | | 4.71 (t, J = 7.1 Hz, 1H), 2.50-2.44 (m, 1H), |
| | | | 2.27-2.23 (m, 5H), 1.81-1.72 (m, 1H), |
| | | | 1.53-1.50 (m, 2H), 1.19-1.16 (m, 2H) |
| 502 | 467.5 | 1.8 | |
| 503 | 464.3 | 1.63 | |
| 504 | 453.3 | 1.76 | |
| 505 | 453.5 | 2 | |
| 506 | 439.5 | 1.68 | |
| 507 | 438.3 | 1.43 | |
| 508 | 467.3 | 1.91 | H NMR (400 MHz, DMSO) 8.98 (s, 1H), |
| | | | 7.90-7.88 (m, 2H), 7.72 (d, J = 8.5 Hz, 1H), |
| | | | 7.56-7.53 (m, 2H), 7.40-7.33 (m, 3H), 2.56 (s, 3H), |
| | | | 2.23 (s, 3H), 1.52-1.50 (m, 2H), 1.18-1.15 (m, 2H) |
| 509 | 415 | 1.78 | |
| 510 | 462.3 | 1.76 | |
| 511 | 473.1 | 2.07 | |
| 512 | 423.3 | 2.12 | |
| 513 | 516.5 | 1.79 | |
| 514 | 535.5 | 1.45 | |
| 515 | 480.3 | 1.68 | |
| 516 | 493.2 | 1.8 | |
| 517 | 576.5 | 1.71 | |
| 518 | 413 | 1.79 | |
| 519 | 453.1 | 1.89 | |
| 520 | 575.3 | 2.21 | |
| 521 | 402.7 | 1.53 | |
| 522 | 373.5 | 1.84 | |
| 523 | 453.1 | 1.37 | |
| 524 | 516.5 | 1.82 | |
| 525 | 466.5 | 1.98 | |
| 526 | 466.5 | 1.95 | |
| 527 | 452.3 | 1.69 | |
| 528 | 389.5 | 1.61 | |

### II.C.2. Compound of Formula C1

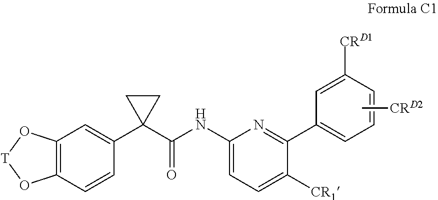

### 1. Embodiments of the Compounds of Formula C1

In one embodiment, in the compound of Formula C1 of the composition

### T is —CH₂—, —CH₂CH₂—, —CF₂—, —C(CH₃)₂—, or —C(O)—;

CR₁′ is H, C₁₋₆ aliphatic, halo, CF₃, CHF₂, O(C₁₋₆ aliphatic); and

### CR^{D1} or CR^{D2} is Z^{D}CR₉

wherein:
Z^{D} is a bond, CONH, SO₂NH, SO₂N(C₁₋₆ alkyl), CH₂NHSO₂, CH₂N(CH₃)SO₂, CH₂NHCO, COO, SO₂, or CO; and CR₉ is H, C₁₋₆ aliphatic, or aryl.

### II.C.2. Compound 2 of Formula C1

In another embodiment, the compound of Formula C1 is Compound 2, depicted below, which is also known by its chemical name 3-(6-(1-(2,2-Difluorobenzo[d][1,3]dioxol-5-yl)cyclopropanecarboxamido)-3-methylpyridin-2-yl)benzoic acid.

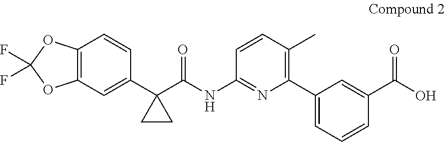 [SMILES: Cc1ccc(NC(=O)C2(c3ccc4c(c3)OC(F)(F)O4)CC2)nc1-c1cccc(C(=O)O)c1]

### 1. Synthesis of Compounds of Formula C1

Compound 2 can be prepared by coupling an acid chloride moiety with an amine moiety according to following Schemes 2-1 to 2-3.

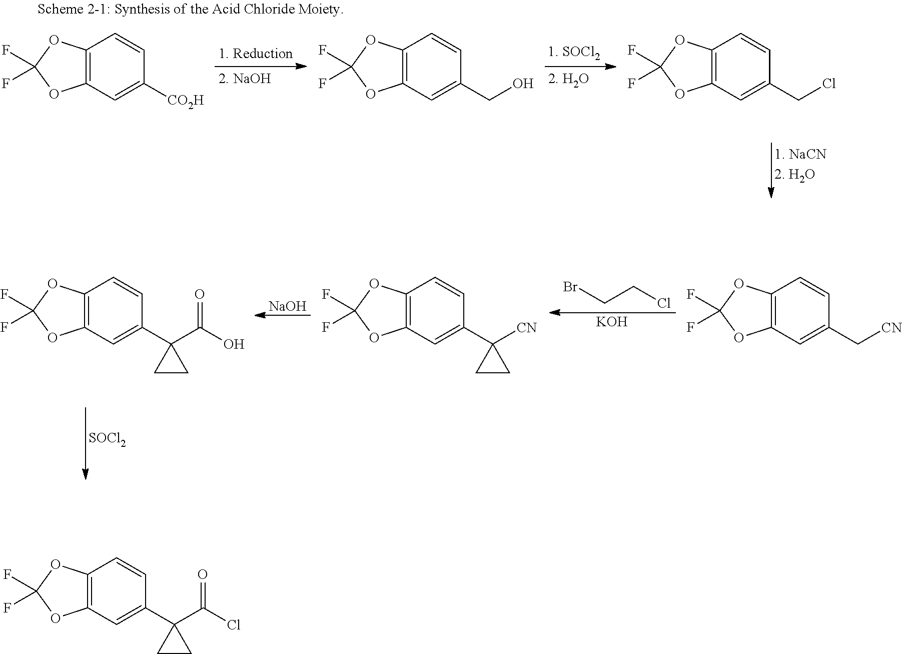

Scheme 2-1 depicts the preparation of 1-(2,2-difluorobenzo[d][1,3]dioxol-5-yl)cyclopropanecarbonyl chloride, which is used in Scheme 2-3 to make the amide linkage of Compound 2.

The starting material, 2,2-difluorobenzo[d][1,3]dioxole-5-carboxylic acid, is commercially available from Saltigo (an affiliate of the Lanxess Corporation). Reduction of the carboxylic acid moiety in 2,2-difluorobenzo[d][1,3]dioxole-5-carboxylic acid to the primary alcohol, followed by conversion to the corresponding chloride using thionyl chloride (SOCl₂), provides 5-(chloromethyl)-2,2-difluorobenzo[d][1,3]dioxole, which is subsequently converted to 2-(2,2-difluorobenzo[d][1,3]dioxol-5-yl)acetonitrile using sodium cyanide. Treatment of 2-(2,2-difluorobenzo[d][1,3]dioxol-5-yl)acetonitrile with base and 1-bromo-2-chloroethane provides 1-(2,2-difluorobenzo[d][1,3]dioxol-5-yl)cyclopropanecarbonitrile. The nitrile moiety in 1-(2,2-difluorobenzo[d][1,3]dioxol-5-yl)cyclopropanecarbonitrile is converted to a carboxylic acid using base to give 1-(2,2-difluorobenzo[d][1,3]dioxol-5-yl)cyclopropanecarboxylic acid, which is converted to the desired acid chloride using thionyl chloride.

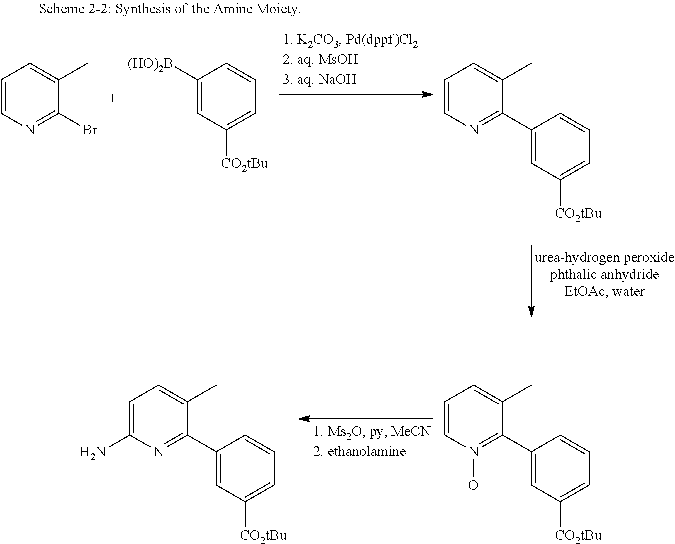

Scheme 2-2 depicts the preparation of the requisite tert-butyl 3-(6-amino-3-methylpyridin-2-yl)benzoate, which is coupled with 1-(2,2-difluorobenzo[d][1,3]dioxol-5-yl)cyclopropanecarbonyl chloride in Scheme 3-3 to give Compound 2. Palladium-catalyzed coupling of 2-bromo-3-methylpyridine with 3-(tert-butoxycarbonyl)phenylboronic acid gives tert-butyl 3-(3-methylpyridin-2-yl)benzoate, which is subsequently converted to the desired compound.

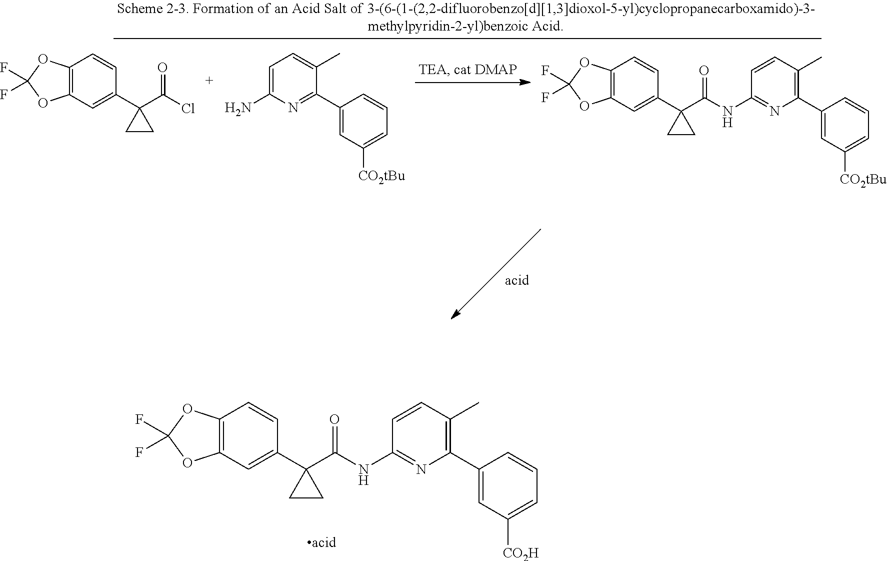

Scheme 2-3 depicts the coupling of 1-(2,2-difluorobenzo[d][1,3]dioxol-5-yl)cyclopropanecarbonyl chloride with tert-butyl 3-(6-amino-3-methylpyridin-2-yl)benzoate using triethyl amine and 4-dimethylaminopyridine to initially provide the tert-butyl ester of Compound 2. Treatment of the tert-butyl ester with an acid such as HCl, gives the HCl salt of Compound 2, which is typically a crystalline solid.

### Experimentals

Vitride® (sodium bis(2-methoxyethoxy)aluminum hydride [or NaAlH₂(OCH₂CH₂OCH₃)₂], 65 wgt % solution in toluene) was purchased from Aldrich Chemicals.

2,2-Difluoro-1,3-benzodioxole-5-carboxylic acid was purchased from Saltigo (an affiliate of the Lanxess Corporation).

### (2,2-Difluoro-1,3-benzodioxol-5-yl)-methanol

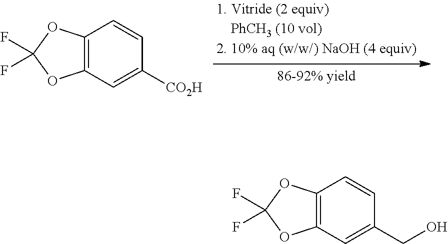 [SMILES: O=C(O)c1ccc2c(c1)OC(F)(F)O2 ; OCc1ccc2c(c1)OC(F)(F)O2]

Commercially available 2,2-difluoro-1,3-benzodioxole-5-carboxylic acid (1.0 eq) was slurried in toluene (10 vol). Vitride® (2 eq) was added via addition funnel at a rate to maintain the temperature at 15-25° C. At the end of the addition, the temperature was increased to 40° C. for 2 hours (h), then 10% (w/w) aqueous (aq) NaOH (4.0 eq) was carefully added via addition funnel, maintaining the temperature at 40-50° C. After stirring for an additional 30 minutes (min), the layers were allowed to separate at 40° C. The organic phase was cooled to 20° C., then washed with water (2×1.5 vol), dried (Na₂SO₄), filtered, and concentrated to afford crude (2,2-difluoro-1,3-benzodioxol-5-yl)-methanol that was used directly in the next step.

### 5-Chloromethyl-2,2-difluoro-1,3-benzodioxole

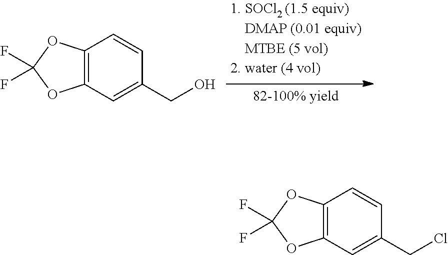 [SMILES: OCc1ccc2c(c1)OC(F)(F)O2 ; FC1(F)Oc2ccc(CCl)cc2O1]

(2,2-difluoro-1,3-benzodioxol-5-yl)-methanol (1.0 eq) was dissolved in MTBE (5 vol). A catalytic amount of 4-(N,N-dimethyl)aminopyridine (DMAP) (1 mol %) was added and SOCl₂ (1.2 eq) was added via addition funnel. The SOCl₂ was added at a rate to maintain the temperature in the reactor at 15-25° C. The temperature was increased to 30° C. for 1 h, and then was cooled to 20° C. Water (4 vol) was added via addition funnel while maintaining the temperature at less than 30° C. After stirring for an additional 30 min, the layers were allowed to separate. The organic layer was stirred and 10% (w/v) aq NaOH (4.4 vol) was added. After stirring for 15 to 20 min, the layers were allowed to separate. The organic phase was then dried (Na₂SO₄), filtered, and concentrated to afford crude 5-chloromethyl-2,2-difluoro-1,3-benzodioxole that was used directly in the next step.

### (2,2-Difluoro-1,3-benzodioxol-5-yl)-acetonitrile

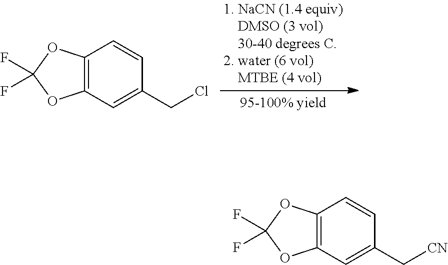 [SMILES: FC1(F)Oc2ccc(CCl)cc2O1 ; N#CCc1ccc2c(c1)OC(F)(F)O2]

A solution of 5-chloromethyl-2,2-difluoro-1,3-benzodioxole (1 eq) in DMSO (1.25 vol) was added to a slurry of NaCN (1.4 eq) in DMSO (3 vol), while maintaining the temperature between 30-40° C. The mixture was stirred for 1 h, and then water (6 vol) was added, followed by methyl tert-butyl ether (MTBE) (4 vol). After stirring for 30 min, the layers were separated. The aqueous layer was extracted with MTBE (1.8 vol). The combined organic layers were washed with water (1.8 vol), dried (Na₂SO₄), filtered, and concentrated to afford crude (2,2-difluoro-1,3-benzodioxol-5-yl)-acetonitrile (95%) that was used directly in the next step.

### (2,2-Difluoro-1,3-benzodioxol-5-yl)-cyclopropanecarbonitrile

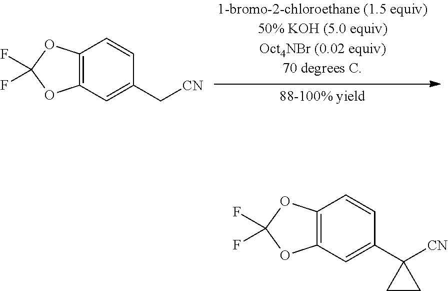 [SMILES: N#CCc1ccc2c(c1)OC(F)(F)O2 ; N#CC1(c2ccc3c(c2)OC(F)(F)O3)CC1]

A mixture of (2,2-difluoro-1,3-benzodioxol-5-yl)-acetonitrile (1.0 eq), 50 wt % aqueous KOH (5.0 eq) 1-bromo-2-chloroethane (1.5 eq), and Oct₄NBr (0.02 eq) was heated at 70° C. for 1 h. The reaction mixture was cooled, then worked up with MTBE and water. The organic phase was washed with water and brine. The solvent was removed to afford (2,2-difluoro-1,3-benzodioxol-5-yl)-cyclopropanecarbonitrile.

### 1-(2,2-Difluoro-1,3-benzodioxol-5-yl)-cyclopropanecarboxylic acid

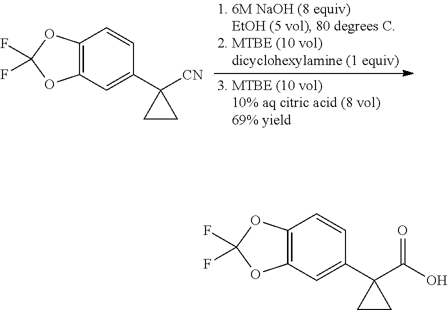 [SMILES: N#CC1(c2ccc3c(c2)OC(F)(F)O3)CC1 ; O=C(O)C1(c2ccc3c(c2)OC(F)(F)O3)CC1]

(2,2-difluoro-1,3-benzodioxol-5-yl)-cyclopropanecarbonitrile was hydrolyzed using 6 M NaOH (8 equiv) in ethanol (5 vol) at 80° C. overnight. The mixture was cooled to room temperature and the ethanol was evaporated under vacuum. The residue was taken up in water and MTBE, 1 M HCl was added, and the layers were separated. The MTBE layer was then treated with dicyclohexylamine (DCHA) (0.97 equiv). The slurry was cooled to 0° C., filtered and washed with heptane to give the corresponding DCHA salt. The salt was taken into MTBE and 10% citric acid and stirred until all the solids had dissolved. The layers were separated and the MTBE layer was washed with water and brine. A solvent swap to heptane followed by filtration gave 1-(2,2-difluoro-1,3-benzodioxol-5-yl)-cyclopropanecarboxylic acid after drying in a vacuum oven at 50° C. overnight.

### 1-(2,2-Difluoro-1,3-benzodioxol-5-yl)-cyclopropanecarbonyl chloride

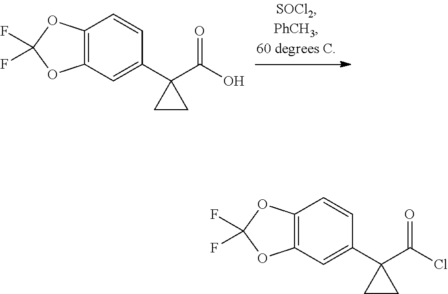 [SMILES: O=C(O)C1(c2ccc3c(c2)OC(F)(F)O3)CC1 ; O=C(Cl)C1(c2ccc3c(c2)OC(F)(F)O3)CC1]

1-(2,2-difluoro-1,3-benzodioxol-5-yl)-cyclopropanecarboxylic acid (1.2 eq) is slurried in toluene (2.5 vol) and the mixture was heated to 60° C. SOCl₂ (1.4 eq) was added via addition funnel. The toluene and SOCl₂ were distilled from the reaction mixture after 30 minutes. Additional toluene (2.5 vol) was added and the resulting mixture was distilled again, leaving the product acid chloride as an oil, which was used without further purification.

### tert-Butyl-3-(3-methylpyridin-2-yl)benzoate

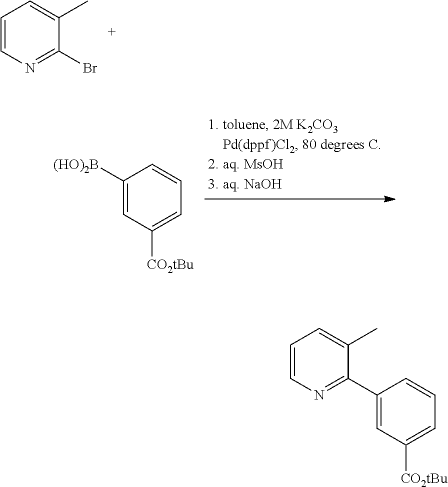

2-Bromo-3-methylpyridine (1.0 eq) was dissolved in toluene (12 vol). K₂CO₃ (4.8 eq) was added, followed by water (3.5 vol). The resulting mixture was heated to 65° C. under a stream of N₂ for 1 hour. 3-(t-Butoxycarbonyl)phenylboronic acid (1.05 eq) and Pd(dppf)Cl₂.CH₂Cl₂ (0.015 eq) were then added and the mixture was heated to 80° C. After 2 hours, the heat was turned off, water was added (3.5 vol), and the layers were allowed to separate. The organic phase was then washed with water (3.5 vol) and extracted with 10% aqueous methanesulfonic acid (2 eq MsOH, 7.7 vol). The aqueous phase was made basic with 50% aqueous NaOH (2 eq) and extracted with EtOAc (8 vol). The organic layer was concentrated to afford crude tert-butyl-3-(3-methylpyridin-2-yl)benzoate (82%) that was used directly in the next step.

### 2-(3-(tert-Butoxycarbonyl)phenyl)-3-methylpyridine-1-oxide

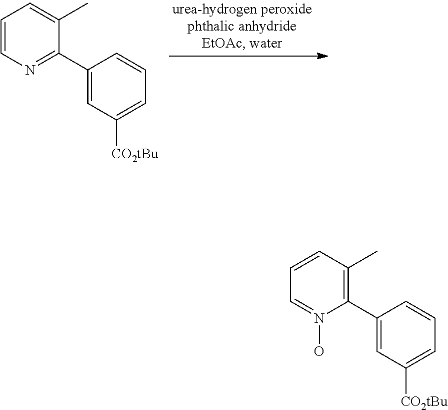

Tert-butyl-3-(3-methylpyridin-2-yl)benzoate (1.0 eq) was dissolved in EtOAc (6 vol). Water (0.3 vol) was added, followed by urea-hydrogen peroxide (3 eq). Phthalic anhydride (3 eq) was then added portionwise to the mixture as a solid at a rate to maintain the temperature in the reactor below 45° C. After completion of the phthalic anhydride addition, the mixture was heated to 45° C. After stirring for an additional 4 hours, the heat was turned off. 10% w/w aqueous Na₂SO₃ (1.5 eq) was added via addition funnel. After completion of Na₂SO₃ addition, the mixture was stirred for an additional 30 min and the layers separated. The organic layer was stirred and 10% wt/wt aqueous. Na₂CO₃ (2 eq) was added. After stirring for 30 minutes, the layers were allowed to separate. The organic phase was washed 13% w/v aq NaCl. The organic phase was then filtered and concentrated to afford crude 2-(3-(tert-butoxycarbonyl)phenyl)-3-methylpyridine-1-oxide (95%) that was used directly in the next step.

### tert-Butyl-3-(6-amino-3-methylpyridin-2-yl)benzoate

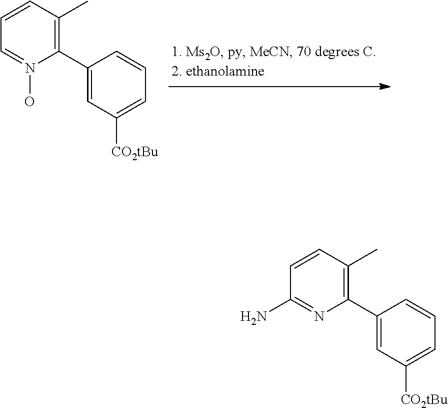

A solution of 2-(3-(tert-butoxycarbonyl)phenyl)-3-methylpyridine-1-oxide (1 eq) and pyridine (4 eq) in acetonitrile (8 vol) was heated to 70° C. A solution of methanesulfonic anhydride (1.5 eq) in MeCN (2 vol) was added over 50 min via addition funnel while maintaining the temperature at less than 75° C. The mixture was stirred for an additional 0.5 hours after complete addition. The mixture was then allowed to cool to ambient. Ethanolamine (10 eq) was added via addition funnel. After stirring for 2 hours, water (6 vol) was added and the mixture was cooled to 10° C. After stirring for 3 hours, the solid was collected by filtration and washed with water (3 vol), 2:1 acetonitrile/water (3 vol), and acetonitrile (2×1.5 vol). The solid was dried to constant weight (<1% difference) in a vacuum oven at 50° C. with a slight N₂ bleed to afford tert-butyl-3-(6-amino-3-methylpyridin-2-yl)benzoate as a red yellow solid (53% yield).

### 3-(6-(1-(2,2-Difluorobenzo[d][1,3]dioxol-5-yl)-cyclopropanecarboxamido)-3-methylpyridin-2-yl)-t-butylbenzoate

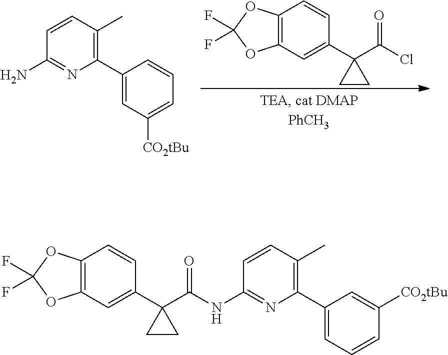 [SMILES: Cc1ccc(N)nc1-c1cccc(C(=O)OC(C)(C)C)c1 ; O=C(Cl)C1(c2ccc3c(c2)OC(F)(F)O3)CC1 ; Cc1ccc(NC(=O)C2(c3ccc4c(c3)OC(F)(F)O4)CC2)nc1-c1cccc(C(=O)OC(C)(C)C)c1]

The crude acid chloride described above was dissolved in toluene (2.5 vol based on acid chloride) and added via addition funnel to a mixture of tert-butyl-3-(6-amino-3-methylpyridin-2-yl)benzoate (1 eq), DMAP, (0.02 eq), and triethylamine (3.0 eq) in toluene (4 vol based on tert-butyl-3-(6-amino-3-methylpyridin-2-yl)benzoate). After 2 hours, water (4 vol based on tert-butyl-3-(6-amino-3-methylpyridin-2-yl)benzoate) was added to the reaction mixture. After stirring for 30 minutes, the layers were separated. The organic phase was then filtered and concentrated to afford a thick oil of 3-(6-(1-(2,2-difluorobenzo[d][1,3]dioxol-5-yl)cyclopropanecarboxamido)-3-methylpyridin-2-yl)-t-butylbenzoate (quantitative crude yield). Acetonitrile (3 vol based on crude product) was added and distilled until crystallization occurs. Water (2 vol based on crude product) was added and the mixture stirred for 2 h. The solid was collected by filtration, washed with 1:1 (by volume) acetonitrile/water (2×1 volumes based on crude product), and partially dried on the filter under vacuum. The solid was dried to a constant weight (<1% difference) in a vacuum oven at 60° C. with a slight N₂ bleed to afford 3-(6-(1-(2,2-difluorobenzo[d][1,3]dioxol-5-yl)cyclopropanecarboxamido)-3-methylpyridin-2-yl)-t-butylbenzoate as a brown solid.

### 3-(6-(1-(2,2-Difluorobenzo[d][1,3]dioxol-5-yl)cyclopropanecarboxamido)-3-methylpyridin-2-yl)benzoic acid.HCl salt

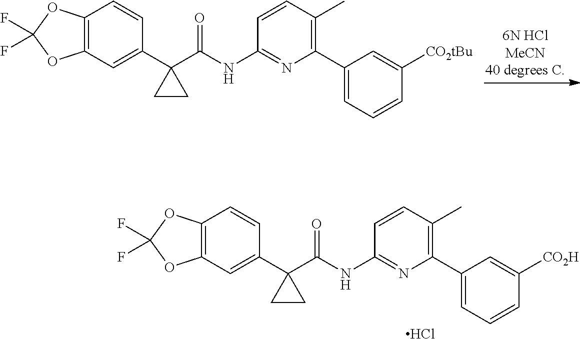 [SMILES: Cc1ccc(NC(=O)C2(c3ccc4c(c3)OC(F)(F)O4)CC2)nc1-c1cccc(C(=O)OC(C)(C)C)c1 ; Cc1ccc(NC(=O)C2(c3ccc4c(c3)OC(F)(F)O4)CC2)nc1-c1cccc(C(=O)O)c1 ; Cl]

To a slurry of 3-(6-(1-(2,2-difluorobenzo[d][1,3]dioxol-5-yl)cyclopropanecarboxamido)-3-methylpyridin-2-yl)-t-butylbenzoate (1.0 eq) in MeCN (3.0 vol) was added water (0.83 vol) followed by concentrated aqueous HCl (0.83 vol). The mixture was heated to 45±5° C. After stirring for 24 to 48 h, the reaction was complete, and the mixture was allowed to cool to ambient. Water (1.33 vol) was added and the mixture stirred. The solid was collected by filtration, washed with water (2×0.3 vol), and partially dried on the filter under vacuum. The solid was dried to a constant weight (<1% difference) in a vacuum oven at 60° C. with a slight N₂ bleed to afford 3-(6-(1-(2,2-difluorobenzo[d][1,3]dioxol-5-yl)cyclopropanecarboxamido)-3-methylpyridin-2-yl)benzoic acid.HCl as an off-white solid.

Table II.C-6 below recites physical data for Compound 2.

TABLE II.C-6

| | LC/MS | LC/RT | |
|---|---|---|---|
| Compound | M + 1 | minutes | NMR |
| Compound 2 | 453.3 | 1.93 | ¹HNMR (400 MHz, DMSO-d6) δ 9.14 |
| | | | (s, 1H), 7.99-7.93 (m, 3H), 7.80-7.78 |
| | | | (m, 1H), 7.74-7.72 (m, 1H), 7.60-7.55 |
| | | | (m, 2H), 7.41-7.33 (m, 2H), 2.24 (s, |
| | | | 3H), 1.53-1.51 (m, 2H), 1.19-1.17 |
| | | | (m, 2H). |

### II.D Embodiments of Column D Compounds

### II.D.1 Compounds of Formula D

The present invention relates to compounds of Formula D, which are useful as modulators of ABC transporter activity:

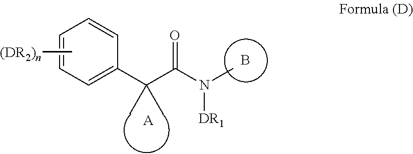

or a pharmaceutically acceptable salt thereof. The modulators of ABC transporter activity in Column D are fully described and exemplified in U.S. Pat. Nos. 7,645,789 and 7,776,905, which are commonly assigned to the Assignee of the present invention. All of the compounds recited in the above patents are useful in the present invention and are hereby incorporated into the present disclosure in their entirety.

DR₁ is —Z^{A}DR₄, wherein each Z^{A} is independently a bond or an optionally substituted branched or straight C₁₋₆ aliphatic chain wherein up to two carbon units of Z^{A} are optionally and independently replaced by —CO—, —CS—, —CONDR^{A}—, —CONDR^{A}NDR^{A}—, —CO₂—, —OCO—, —NDR^{A}CO₂—, —O—, —NDR^{A}CONDR^{A}—, —OCONDR^{A}—, —NDR^{A}NDR^{A}—, —NDR^{A}CO—, —S—, —SO—, —SO₂—, —NDR^{A}—, —SO₂NDR^{A}—, —NDR^{A}SO₂—, or —NDR^{A}SO₂NDR^{A}—. Each DR₄ is independently DR^{A}, halo, —OH, —NH₂, —NO₂, —CN, or —OCF₃. Each DR^{A} is independently hydrogen, an optionally substituted aliphatic, an optionally substituted cycloaliphatic, an optionally substituted heterocycloaliphatic, an optionally substituted aryl, or an optionally substituted heteroaryl.

DR₂ is —Z^{B}DR₅, wherein each Z^{B} is independently a bond or an optionally substituted branched or straight C₁₋₆ aliphatic chain wherein up to two carbon units of Z^{B} are optionally and independently replaced by —CO—, —CS—, —CONDR^{B}—, —CONDR^{B}NDR^{B}—, —CO₂—, —OCO—, —NDR^{B}CO₂—, —O—, —NDR^{B}CONDR^{B}—, —OCONDR^{B}—, —NDR^{B}NDR^{B}—, —NDR^{B}CO—, —S—, —SO—, —SO₂—, —NDR^{B}—, —SO₂NDR^{B}—, —NDR^{B}SO₂—, or —NDR^{B}SO₂NDR^{B}—. Each DR₅ is independently DR^{B}, halo, —OH, —NH₂, —NO₂, —CN, —CF₃, or —OCF₃. Each DR^{B} is independently hydrogen, an optionally substituted aliphatic, an optionally substituted cycloaliphatic, an optionally substituted heterocycloaliphatic, an optionally substituted aryl, or an optionally substituted heteroaryl. Alternatively, any two adjacent DR₂ groups together with the atoms to which they are attached form an optionally substituted carbocycle or an optionally substituted heterocycle.

Ring A is an optionally substituted 3-7 membered monocyclic ring having 0-3 heteroatoms selected from N, O, and S.

Ring B is a group having formula DIa:

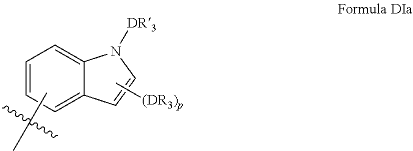

or a pharmaceutically acceptable salt thereof, wherein p is 0-3 and each DR₃ and DR′₃ is independently —Z^{C}DR₆, where each Z^{C} is independently a bond or an optionally substituted branched or straight C₁₋₆ aliphatic chain wherein up to two carbon units of Z^{C} are optionally and independently replaced by —CO—, —CS—, —CONDR^{C}—, —CONDR^{C}NDR^{C}—, —CO₂—, —OCO—, —NDR^{C}CO₂—, —O—, —NDR^{C}CONDR^{C}—, —OCONDR^{C}—, —NDR^{C}NDR^{C}—, —NDR^{C}CO—, —S—, —SO—, —SO₂—, —NDR^{C}—, —SO₂NDR^{C}—, —NDR^{C}SO₂—, or —NDR^{C}SO₂NR^{C}—. Each DR₆ is independently DR^{C}, halo, —OH, —NH₂, —NO₂, —CN, or —OCF₃. Each DR^{C} is independently hydrogen, an optionally substituted aliphatic, an optionally substituted cycloaliphatic, an optionally substituted heterocycloaliphatic, an optionally substituted aryl, or an optionally substituted heteroaryl. Alternatively, any two adjacent DR₃ groups together with the atoms to which they are attached form an optionally substituted carbocycle or an optionally substituted heterocycle. Furthermore, DR′₃ and an adjacent DR₃ group, together with the atoms to which they are attached, form an optionally substituted heterocycle.

n is 1-3.

However, in several embodiments, when ring A is unsubstituted cyclopentyl, n is 1, DR₂ is 4-chloro, and DR₁ is hydrogen, then ring B is not 2-(tertbutyl)indol-5-yl, or (2,6-dichlorophenyl(carbonyl))-3-methyl-1H-indol-5-yl; and when ring A is unsubstituted cyclopentyl, n is 0, and DR₁ is hydrogen, then ring B is not

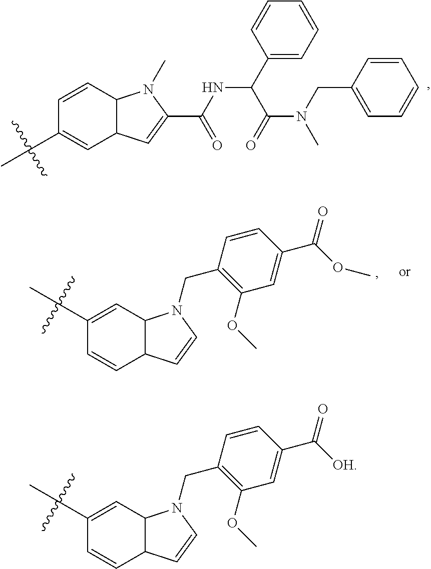 [SMILES: *C1=CC2C=C(C(=O)NC(C(=O)N(C)Cc3ccccc3)c3ccccc3)N(C)C2C=C1 ; *C1=CC2C(C=C1)C=CN2Cc1ccc(C(=O)OC)cc1OC ; *C1=CC2C(C=C1)C=CN2Cc1ccc(C(=O)O)cc1OC]

B. Specific Compounds

1. DR₁ Group

DR₁ is —Z^{A}DR₄, wherein each Z^{A} is independently a bond or an optionally substituted branched or straight C₁₋₆ aliphatic chain wherein up to two carbon units of Z^{A} are optionally and independently replaced by —CO—, —CS—, —CONDR^{A}—, —CONDR^{A}NDR^{A}—, —CO₂—, —OCO—, —NDR^{A}CO₂—, —O—, —NDR^{A}CONDR^{A}—, —OCONDR^{A}—, —NDR^{A}NDR^{A}—, —NDR^{A}CO—, —S—, —SO—, —SO₂—, —NDR^{A}—, —SO₂NDR^{A}—, —NDR^{A}SO₂—, or —NDR^{A}SO₂NDR^{A}—. Each DR₄ is independently DR^{A}, halo, —OH, —NH₂, —NO₂, —CN, or —OCF₃. Each DR^{A} is independently hydrogen, an optionally substituted aliphatic, an optionally substituted cycloaliphatic, an optionally substituted heterocycloaliphatic, an optionally substituted aryl, or an optionally substituted heteroaryl.

In several embodiments, DR₁ is —Z^{A}DR₄, wherein each Z^{A} is independently a bond or an optionally substituted branched or straight C₁₋₆ aliphatic chain and each DR₄ is hydrogen.

In other embodiments, DR₁ is —Z^{A}DR₄, wherein each Z^{A} is a bond and each DR₄ is hydrogen.

2. DR₂ Group

Each DR₂ is independently —Z^{B}DR₅, wherein each Z^{B} is independently a bond or an optionally substituted branched or straight C₁₋₆ aliphatic chain wherein up to two carbon units of Z^{B} are optionally and independently replaced by —CO—, —CS—, —CONDR^{B}—, —CONDR^{B}NDR^{B}—, —CO₂—, —OCO—, —NDR^{B}CO₂—, —O—, —NDR^{B}CONDR^{B}—, —OCONDR^{B}—, —NDR^{B}NDR^{B}—, —NDR^{B}CO—, —S—, —SO—, —SO₂—, —SO₂NDR^{B}—, —NDR^{B}SO₂—, or —NDR^{B}SO₂NDR^{B}—. Each DR₅ is independently DR^{B}, halo, —OH, —NH₂, —NO₂, —CN, —CF₃, or —OCF₃. Each DR^{B} is independently hydrogen, an optionally substituted aliphatic, an optionally substituted cycloaliphatic, an optionally substituted heterocycloaliphatic, an optionally substituted aryl, or an optionally substituted heteroaryl. Alternatively, any two adjacent DR₂ groups together with the atoms to which they are attached form an optionally substituted carbocycle or an optionally substituted heterocycle, or an optionally substituted heteroaryl.

In several embodiments, DR₂ is an optionally substituted aliphatic. For example, DR₂ is an optionally substituted branched or straight C₁₋₆ aliphatic chain. In other examples, DR₂ is an optionally substituted branched or straight C₁₋₆ alkyl chain, an optionally substituted branched or straight C2-6 alkenyl chain, or an optionally substituted branched or straight C2-6 alkynyl chain. In alternative embodiments, DR₂ is a branched or straight C₁₋₆ aliphatic chain that is optionally substituted with 1-3 of halo, hydroxy, cyano, cycloaliphatic, heterocycloaliphatic, aryl, heteroaryl, or combinations thereof. For example, DR₂ is a branched or straight C₁₋₆ alkyl that is optionally substituted with 1-3 of halo, hydroxy, cyano, cycloaliphatic, heterocycloaliphatic, aryl, heteroaryl, or combinations thereof. In still other examples, DR₂ is a methyl, ethyl, propyl, butyl, isopropyl, or tert-butyl, each of which is optionally substituted with 1-3 of halo, hydroxy, cyano, aryl, heteroaryl, cycloaliphatic, or heterocycloaliphatic. In still other examples, DR₂ is a methyl, ethyl, propyl, butyl, isopropyl, or tert-butyl, each of which is unsubstituted.

In several other embodiments, DR₂ is an optionally substituted branched or straight C₁₋₅ alkoxy. For example, DR₂ is a C₁₋₅ alkoxy that is optionally substituted with 1-3 of hydroxy, aryl, heteroaryl, cycloaliphatic, heterocycloaliphatic, or combinations thereof. In other examples, DR₂ is a methoxy, ethoxy, propoxy, butoxy, or pentoxy, each of which is optionally substituted with 1-3 of hydroxy, aryl, heteroaryl, cycloaliphatic, heterocycloaliphatic, or combinations thereof.

In other embodiments, DR₂ is hydroxy, halo, or cyano.

In several embodiments, DR₂ is —Z^{B}DR₅, and Z^{B} is independently a bond or an optionally substituted branched or straight C₁₋₄ aliphatic chain wherein up to two carbon units of Z^{B} are optionally and independently replaced by —C(O)—, —O—, —S—, —S(O)2-, or —NH—, and DR₅ is DR^{B}, halo, —OH, —NH₂, —NO₂, —CN, —CF₃, or —OCF₃, and DR^{B} is hydrogen or aryl.

In several embodiments, two adjacent DR₂ groups form an optionally substituted carbocycle or an optionally substituted heterocycle. For example, two adjacent DR₂ groups form an optionally substituted carbocycle or an optionally substituted heterocycle, either of which is fused to the phenyl of Formula D, wherein the carbocycle or heterocycle has Formula DIb:

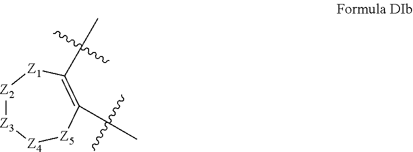

Each of Z₁, Z₂, Z₃, Z₄, and Z₅ is independently a bond, —CDR₇DR′₇—, —NDR₇—, or —O—; each DR₇ is independently —Z^{D}DR₈, wherein each Z^{D} is independently a bind or an optionally substituted branched or straight C₁₋₆ aliphatic chain wherein up to two carbon units of Z^{D} are optionally and independently replaced by —CO—, —CS—, —CONDR^{D}—, —CO₂—, —OCO—, —NDR^{D}CO₂—, —O—, —NDR^{D}CONDR^{D}—, —OCONDR^{D}—, —NDR^{D}NDR^{D}—, —NDR^{D}CO—, —S—, —SO—, —SO₂—, —NDR^{D}—, —SO₂NDR^{D}—, —NDR^{D}SO₂—, or —NDR^{D}SO₂NDR^{D}—. Each DR₈ is independently DR^{D}, halo, —OH, —NH₂, —NO₂, —CN, —CF₃, or —OCF₃. Each DR^{D} is independently hydrogen, an optionally substituted cycloaliphatic, an optionally substituted heterocycloaliphatic, an optionally substituted aryl, or an optionally substituted heteroaryl. Each DR′₇ is independently hydrogen, optionally substituted C₁₋₆ aliphatic, hydroxy, halo, cyano, nitro, or combinations thereof. Alternatively, any two adjacent DR₇ groups together with the atoms to which they are attached form an optionally substituted 3-7 membered carbocyclic ring, such as an optionally substituted cyclobutyl ring, or any two DR₇ and DR′₇ groups together with the atom or atoms to which they are attached form an optionally substituted 3-7 membered carbocyclic ring or a heterocarbocyclic ring.

In several other examples, two adjacent DR₂ groups form an optionally substituted carbocycle. For example, two adjacent DR₂ groups form an optionally substituted 5-7 membered carbocycle that is optionally substituted with 1-3 of halo, hydroxy, cyano, oxo, cyano, alkoxy, alkyl, or combinations thereof. In another example, two adjacent DR₂ groups form a 5-6 membered carbocycle that is optionally substituted with 1-3 of halo, hydroxy, cyano, oxo, cyano, alkoxy, alkyl, or combinations thereof. In still another example, two adjacent DR₂ groups form an unsubstituted 5-7 membered carbocycle.

In alternative examples, two adjacent DR₂ groups form an optionally substituted heterocycle. For instance, two adjacent DR₂ groups form an optionally substituted 5-7 membered heterocycle having 1-3 heteroatoms independently selected from N, O, and S. In several examples, two adjacent DR₂ groups form an optionally substituted 5-6 membered heterocycle having 1-2 oxygen atoms. In other examples, two adjacent DR₂ groups form an unsubstituted 5-7 membered heterocycle having 1-2 oxygen atoms. In other embodiments, two adjacent DR₂ groups form a ring selected from:

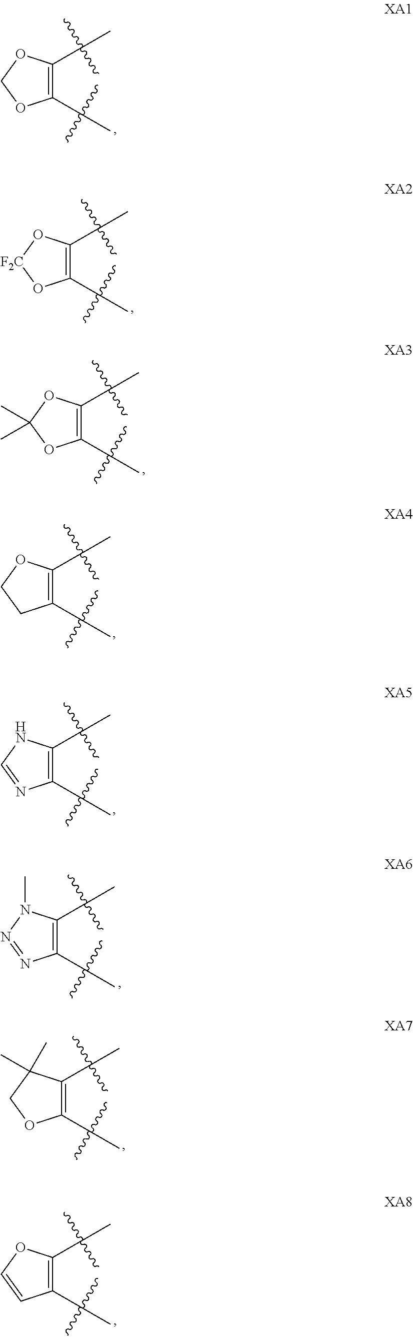

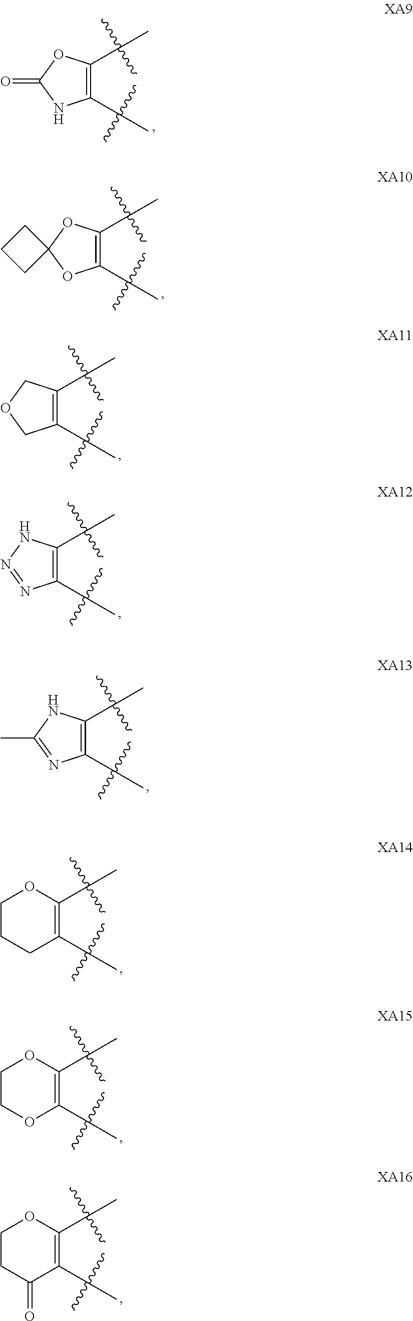

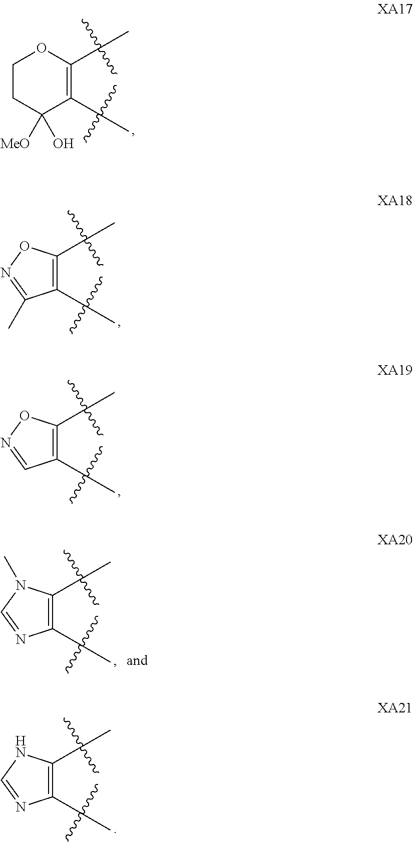

In alternative examples, two adjacent DR₂ groups form an optionally substituted carbocycle or an optionally substituted heterocycle, and a third DR₂ group is attached to any chemically feasible position on the phenyl of formula DI. For instance, an optionally substituted carbocycle or an optionally substituted heterocycle, both of which is formed by two adjacent DR₂ groups; a third DR₂ group; and the phenyl of Formula D form a group having Formula DIc:

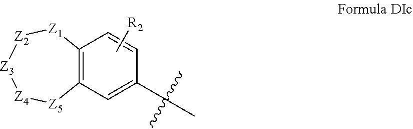

Z₁, Z₂, Z₃, Z₄, and Z₅ has been defined above in Formula DIb, and DR₂ has been defined above in Formula D.

In several embodiments, each DR₂ group is independently selected from hydrogen, halo, —OCH₃, —OH, —CH₂OH, —CH₃, and —OCF₃, and/or two adjacent DR₂ groups together with the atoms to which they are attached form

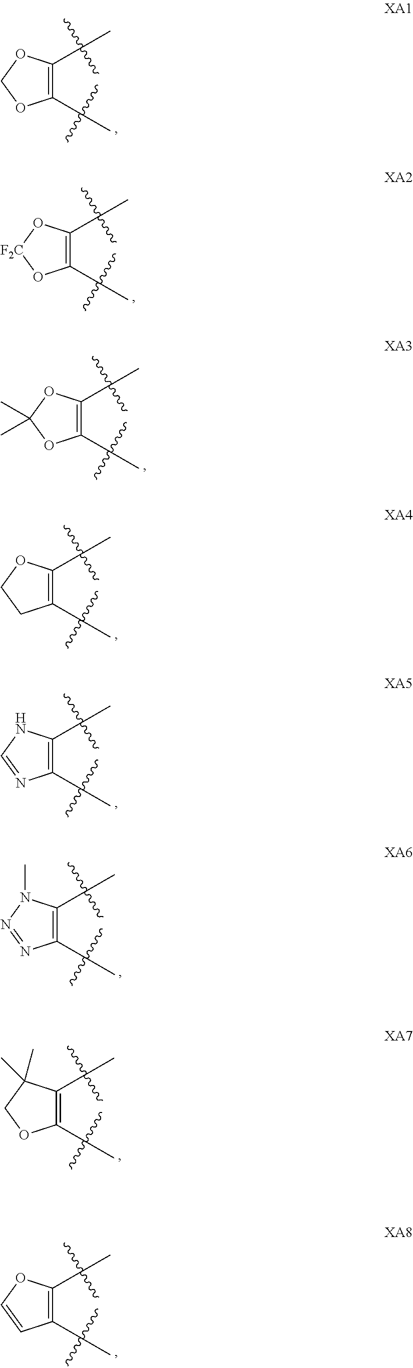

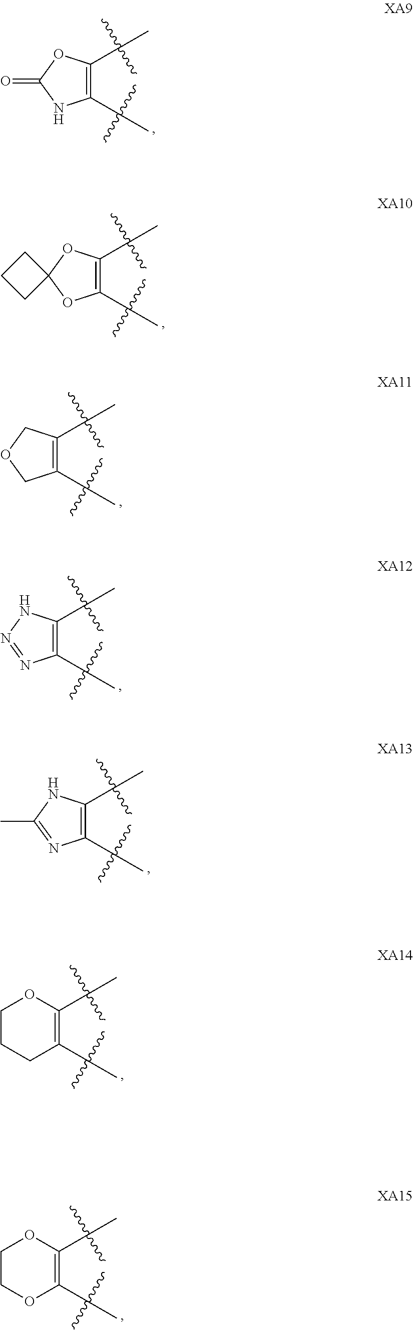

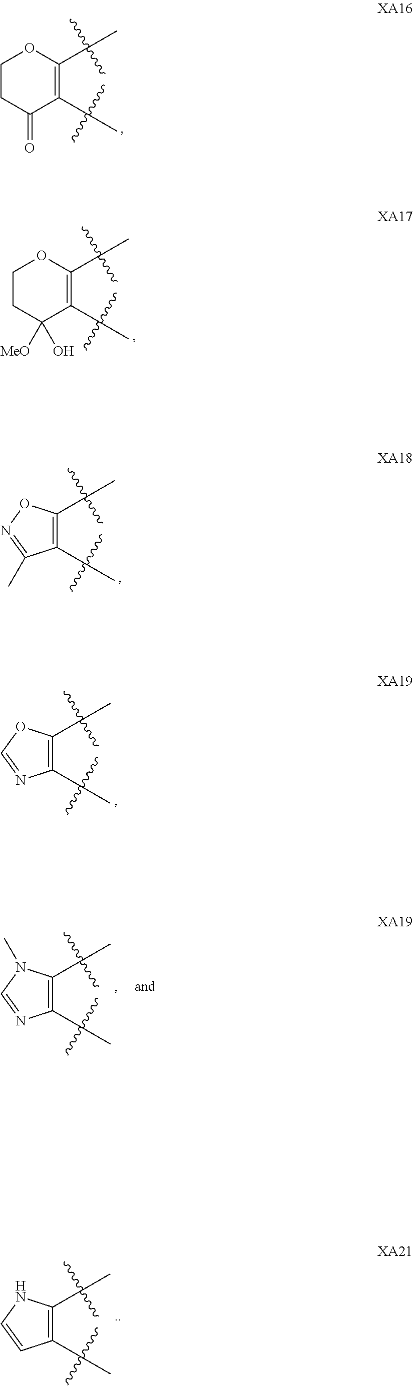

In other embodiments, R₂ is at least one selected from hydrogen, halo, methoxy, phenylmethoxy, hydroxy, hydroxymethyl, trifluoromethoxy, and methyl.

In some embodiments, two adjacent DR₂ groups, together with the atoms to which they are attached, form

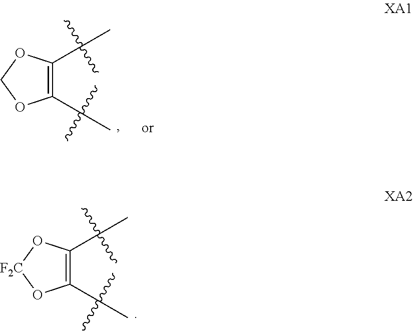 [SMILES: *C1=C(*)OCO1 ; *C1=C(*)OC(F)(F)O1]

3. Ring A

Ring A is an optionally substituted 3-7 membered monocyclic ring having 0-3 heteroatoms selected from N, O, and S.

In several embodiments, ring A is an optionally substituted 3-7 membered monocyclic cycloaliphatic. For example, ring A is a cyclopropyl, cyclobutyl, cyclopentyl, cyclohexyl, or cycloheptyl, each of which is optionally substituted with 1-3 of halo, hydroxy, C₁₋₅ aliphatic, or combinations thereof.

In other embodiments, ring A is an optionally substituted 3-7 membered monocyclic heterocycloaliphatic. For example, ring A is an optionally substituted 3-7 membered monocyclic heterocycloaliphatic having 1-2 heteroatoms independently selected from N, O, and S. In other examples, ring A is tetrahydrofuran-yl, tetrahydro-2H-pyran-yl, pyrrolidone-yl, or piperidine-yl, each of which is optionally substituted.

In still other examples, ring A is selected from

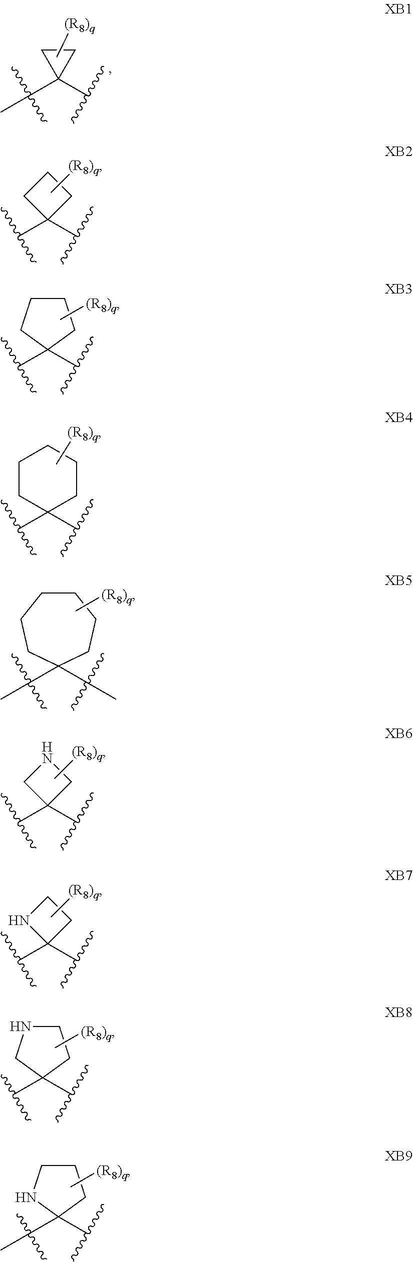

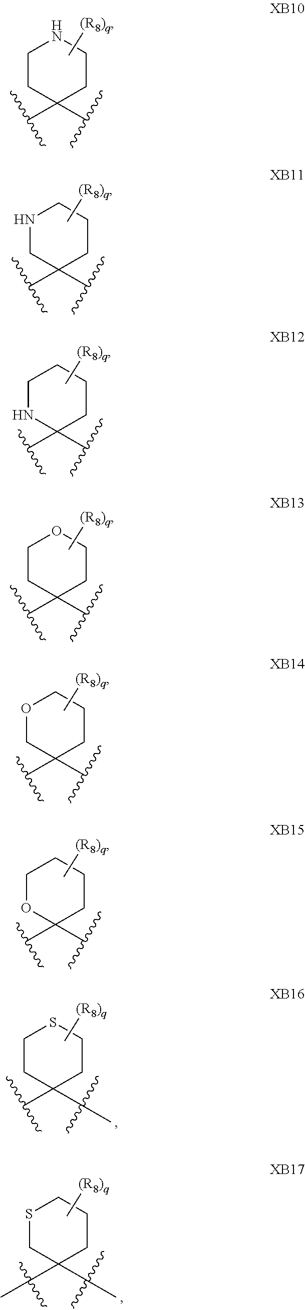

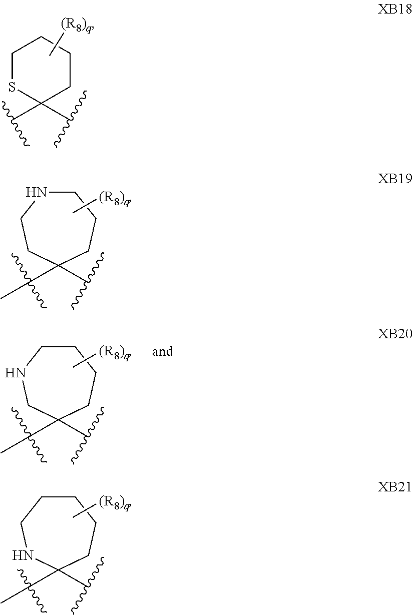

Each DR₃ is independently —Z^{E}DR₉, wherein each Z^{E} is independently a bond or an optionally substituted branched or straight C₁₋₅ aliphatic chain wherein up to two carbon units of Z^{E} are optionally and independently replaced by —CO—, —CS—, —CONDR^{E}—, —CO₂—, —OCO—, —NDR^{E}COᵣ, —O—, —NDR^{E}CONDR^{E}—, —OCONDR^{E}—, —NDR^{E}NDR^{E}—, —NDR^{E}CO—, —S—, —SO—, —SO₂—, —NDR^{E}—, —SO₂NDR^{E}—, —NDR^{E}SO₂—, or —NDR^{E}SO₂NDR^{E}—, each DR₉ is independently DR^{E}, —OH, —NO₂, —CN, —CF₃, oxo, or —OCF₃. Each DR^{E} is independently hydrogen, an optionally substituted cycloaliphatic, an optionally substituted heterocycloaliphatic, an optionally substituted aryl, or an optionally substituted heteroaryl.

q is 0-5.

In other embodiments, ring A is one selected from

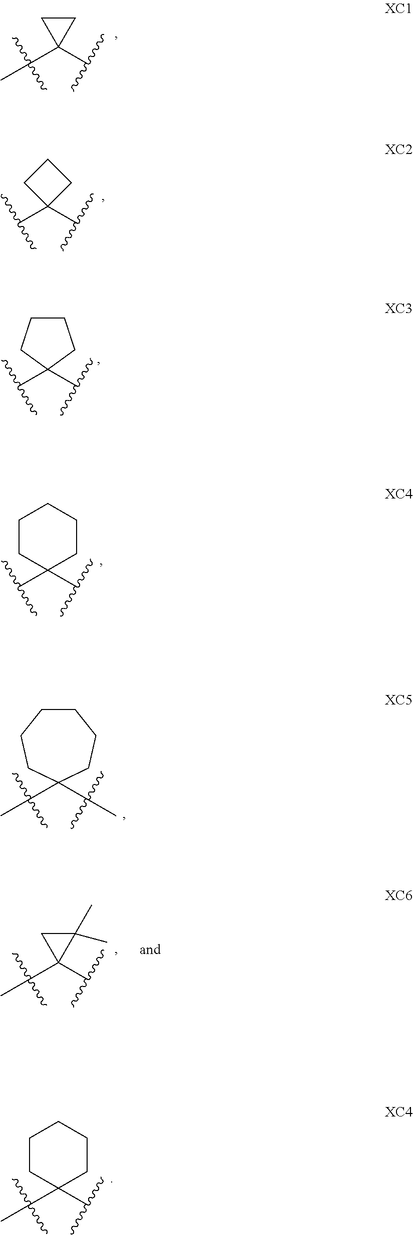

In several embodiments, ring A is

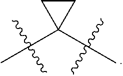 [SMILES: *C1(*)CC1]

4. Ring B

Ring B is a group having Formula DIa:

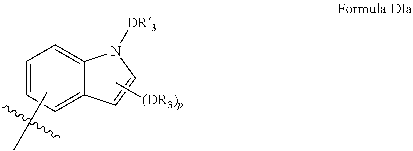

or a pharmaceutically acceptable salt thereof, wherein p is 0-3.

Each DR₃ and DR′₃ is independently —Z^{C}DR₆, where each Z^{C} is independently a bond or an optionally substituted branched or straight C₁₋₆ aliphatic chain wherein up to two carbon units of Z^{C} are optionally and independently replaced by —CO—, —CS—, —CONDR^{C}—, —CONDR^{C}NDR^{C}—, —CO₂—, —OCO—, —NDR^{C}CO₂—, —O—, —NDR^{C}CONDR^{C}—, —OCONDR^{C}—, —NDR^{C}NDR^{C}—, —NDR^{C}CO—, —S—, —SO—, —SO₂—, —SO₂NDR^{C}—, —NDR^{C}SO₂—, or —NDR^{C}SO₂NDR^{C}—. Each DR₆ is independently DR^{C}, halo, —OH, —NH₂, —NO₂, —CN, or —OCF₃. Each DR^{C} is independently hydrogen, an optionally substituted aliphatic, an optionally substituted cycloaliphatic, an optionally substituted heterocycloaliphatic, an optionally substituted aryl, or an optionally substituted heteroaryl. Alternatively, any two adjacent DR₃ groups together with the atoms to which they are attached form an optionally substituted carbocycle or an optionally substituted heterocycle, or DR′₃ and an adjacent DR₃, i.e., attached to the 2 position of the indole of formula DIa, together with the atoms to which they are attached form an optionally substituted heterocycle.

In several embodiments, ring B is

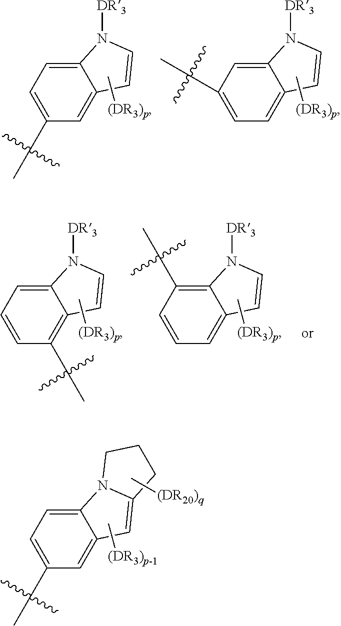

wherein q is 0-3 and each DR₂₀ is —Z^{G}DR₂₁, where each Z^{G} is independently a bond or an optionally substituted branched or straight C₁₋₅ aliphatic chain wherein up to two carbon units of Z^{G} are optionally and independently replaced by —CO—, —CS—, —CONDR^{G}—, —CO₂—, —OCO—, —NDR^{G}CO₂—, —O—, —OCONDR^{G}—, —NDR^{G}NDR^{G}—, —NDR^{G}CO—, —S—, —SO—, —SO₂—, —NDR^{G}—, —SO₂NDR^{G}—, —NDR^{G}SO₂—, or —NDR^{G}SO₂NDR^{G}—. Each DR₂₁ is independently DR^{G}, halo, —OH, —NH₂, —NO₂, —CN, or —OCF₃. Each DR^{G} is independently hydrogen, an optionally substituted aliphatic, an optionally substituted cycloaliphatic, an optionally substituted heterocycloaliphatic, an optionally substituted aryl, or an optionally substituted heteroaryl.

For example, ring B is

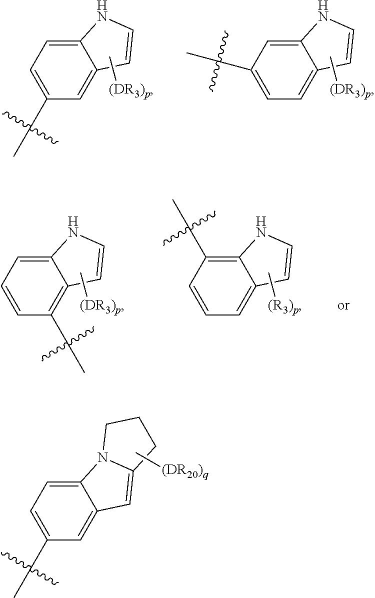

In several embodiments, DR′₃ is hydrogen and DR₃ is attached to the 2, 3, 4, 5, 6, or 7 position of the indole of Formula DIa. In several other examples, DR₃ is attached to the 2 or 3 position of the indole of Formula DIa, and DR₃ is independently an optionally substituted aliphatic. For instance, DR₃ is an optionally substituted acyl group. In several instances, DR₃ is an optionally substituted (alkoxy)carbonyl. In other instances, DR₃ is (methoxy)carbonyl, (ethoxy)carbonyl, (propoxy)carbonyl, or (butoxy)carbonyl, each of which is optionally substituted with 1-3 of halo, hydroxy, or combinations thereof. In other instances, DR₃ is an optionally substituted (aliphatic)carbonyl. For example, DR₃ is an optionally substituted (alkyl)carbonyl that is optionally substituted with 1-3 of halo, hydroxy, or combinations thereof. In other examples, DR₃ is (methyl)carbonyl, (ethyl)carbonyl, (propyl)carbonyl, or (butyl)carbonyl, each of which is optionally substituted with 1-3 of halo, hydroxy, or combinations thereof.

In several embodiments, DR₃ is an optionally substituted (cycloaliphatic)carbonyl or an optionally substituted (heterocycloaliphatic)carbonyl. In several examples, DR₃ is an optionally substituted (C_{3.7} cycloaliphatic)carbonyl. For example, DR₃ is a (cyclopropyl)carbonyl, (cyclobutyl)carbonyl, (cyclopentyl)carbonyl, (cyclohexyl)carbonyl, or (cycloheptyl)carbonyl, each of which is optionally substituted with aliphatic, halo, hydroxy, nitro, cyano, or combinations thereof. In several alternative examples, DR₃ is an optionally substituted (heterocycloaliphatic)carbonyl. For example, DR₃ is an optionally substituted (heterocycloaliphatic)carbonyl having 1-3 heteroatoms independently selected from N, O, and S. In other examples, DR₃ is an optionally substituted (heterocycloaliphatic)carbonyl having 1-3 heteroatoms independently selected from N and O. In still other examples, DR₃ is an optionally substituted 4-7 membered monocyclic (heterocycloaliphatic)carbonyl having 1-3 heteroatoms independently selected from N and O. Alternatively, DR₃ is (piperidine-1-yl,)carbonyl, (pyrrolidine-1-yl)carbonyl, or (morpholine-4-yl)carbonyl, (piperazine-1-yl)carbonyl, each of which is optionally substituted with 1-3 of halo, hydroxy, cyano, nitro, or aliphatic.

In still other instances, DR₃ is optionally substituted (aliphatic)amido such as (aliphatic(amino(carbonyl)) that is attached to the 2 or 3 position on the indole ring of Formula DIa. In some embodiments, DR₃ is an optionally substituted (alkyl(amino))carbonyl that is attached to the 2 or 3 position on the indole ring of Formula DIa. In other embodiments, DR₃ is an optionally substituted straight or branched (aliphatic(amino))carbonyl that is attached to the 2 or 3 position on the indole ring of Formula DIa. In several examples, DR₃ is (N,N-dimethyl(amino))carbonyl, (methyl(amino))carbonyl, (ethyl(amino))carbonyl, (propyl(amino))carbonyl, (prop-2-yl(amino))carbonyl, (dimethyl(but-2-yl(amino)))carbonyl, (tertbutyl(amino))carbonyl, (butyl(amino))carbonyl, each of which is optionally substituted with 1-3 of halo, hydroxy, cycloaliphatic, heterocycloaliphatic, aryl, heteroaryl, or combinations thereof.

In other embodiments, DR₃ is an optionally substituted (alkoxy)carbonyl. For example, DR₃ is (methoxy)carbonyl, (ethoxy)carbonyl, (propoxy)carbonyl, or (butoxy)carbonyl, each of which is optionally substituted with 1-3 of halo, hydroxy, or combinations thereof. In several instances, DR₃ is an optionally substituted straight or branched C₁₋₆ aliphatic. For example, DR₃ is an optionally substituted straight or branched C₁₋₆ alkyl. In other examples, DR₃ is independently an optionally substituted methyl, ethyl, propyl, butyl, isopropyl, or tertbutyl, each of which is optionally substituted with 1-3 of halo, hydroxy, cyano, nitro, or combination thereof. In other embodiments, DR₃ is an optionally substituted C₃₋₆ cycloaliphatic. Exemplary embodiments include cyclopropyl, 1-methyl-cycloprop-1-yl, etc. In other examples, p is 2 and the two DR₃ substituents are attached to the indole of Formula DIa at the 2,4- or 2,6- or 2,7-positions. Exemplary embodiments include 6-F, 3-(optionally substituted C₁₋₆ aliphatic or C₃₋₆ cycloaliphatic); 7-F-2-(−(optionally substituted C₁₋₆ aliphatic or C₃₋₆ cycloaliphatic)), 4F-2-(optionally substituted C₁₋₆ aliphatic or C₃₋₆ cycloaliphatic); 7-CN-2-(optionally substituted C₁₋₆ aliphatic or C₃₋₆ cycloaliphatic); 7-Me-2-(optionally substituted C₁₋₆ aliphatic or C₃₋₆ cycloaliphatic) and 7-OMe-2-(optionally substituted C₁₋₆ aliphatic or C₃₋₆ cycloaliphatic).

In several embodiments, DR₃ is hydrogen. In several instances, DR₃ is an optionally substituted straight or branched C₁₋₆ aliphatic. In other embodiments, DR₃ is an optionally substituted C₃₋₆ cycloaliphatic.

In several embodiments, DR₃ is one selected from:

—H, —CH₃, —CH₂OH, —CH₂CH₃, —CH₂CH₂OH, —CH₂CH₂CH₃, —NH₂, halo, —OCH₃, —CN, —CF₃, —C(O)OCH₂CH₃, —S(O)₂CH₃, —CH₂NH₂, —C(O)NH₂,

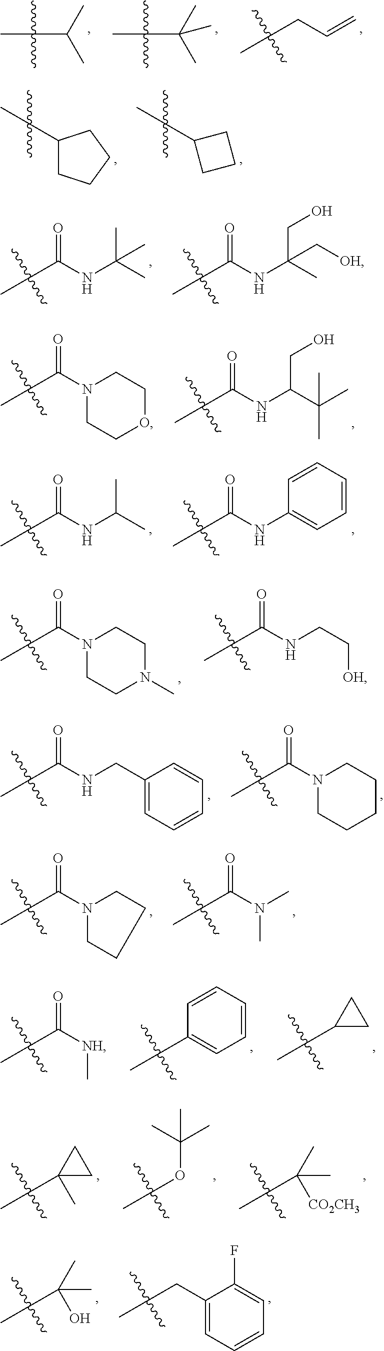

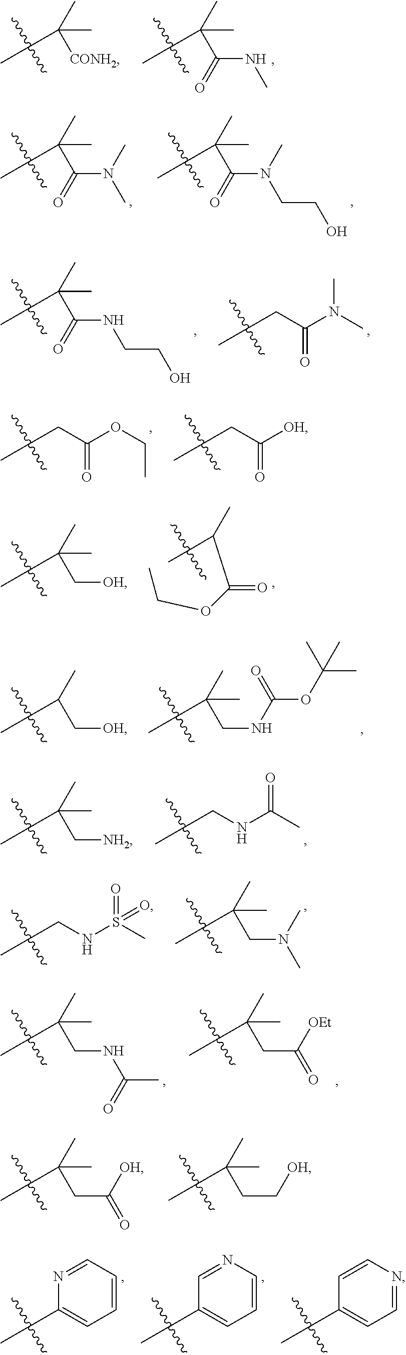

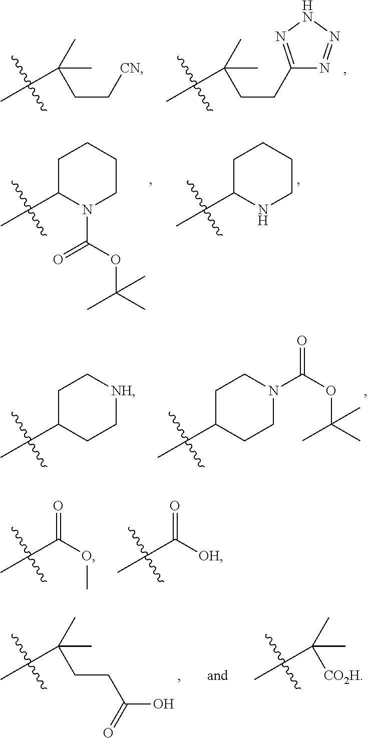

In another embodiment, two adjacent DR₃ groups form

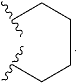 [SMILES: *CCCC*]

In several embodiments, DR′₃ is independently —Z^{C}DR₆, where each Z^{C} is independently a bond or an optionally substituted branched or straight C₁₋₆ aliphatic chain wherein up to two carbon units of Z^{C} are optionally and independently replaced by —CO—, —CS—, —CONDR^{C}—, —CONDR^{C}NDR^{C}—, —CO₂—, —OCO—, —NDR^{C}CO₂—, —O—, —NDR^{C}CONDR^{C}—, —OCONDR^{C}—, —NDR^{C}NDR^{C}—, NDR^{C}CO—, —S—, —SO—, —SO₂—, —NDR^{C}—, —SO₂NDR^{C}—, —NDR^{C}SO₂—, or —NDR^{C}SO₂NDR^{C}—. Each DR₆ is independently DR^{C}, halo, —OH, —NH₂, —NO₂, —CN, or —OCF₃. Each DR^{C} is independently hydrogen, an optionally substituted aliphatic, an optionally substituted cycloaliphatic, an optionally substituted heterocycloaliphatic, or an optionally substituted heteroaryl. In one embodiment, each DR^{C} is hydrogen, C₁₋₆ aliphatic, or C₃₋₆ cycloaliphatic, wherein either of the aliphatic or cycloaliphatic is optionally substituted with up to 4-OH substituents. In another embodiment, DR^{C} is hydrogen, or C₁₋₆ alkyl optionally substituted with up to 4-OH substituents.

For example, in many embodiments, DR′₃ is independently —Z^{C}DR₆, where each Z^{C} is independently a bond or an optionally substituted branched or straight C₁₋₆ aliphatic chain wherein up to two carbon units of Z^{C} are optionally and independently replaced by —C(O)—, —C(O)NDR^{C}—, —C(O)O—, —NDR^{C}C(O)O—, —O—, —NDR^{C}S(O)2-, or —NDR^{C}—. Each DR₆ is independently DR^{C}, —OH, or —NH₂. Each DR^{C} is independently hydrogen, an optionally substituted cycloaliphatic, an optionally substituted heterocycloaliphatic, or an optionally substituted heteroaryl. In one embodiment, each DR^{C} is hydrogen, C₁₋₆ aliphatic, or C₃₋₆ cycloaliphatic, wherein either of the aliphatic or cycloaliphatic is optionally substituted with up to 4-OH substituents. In another embodiment, DR^{C} is hydrogen, or C₁₋₆ alkyl optionally substituted with up to 4-OH substituents.

In other embodiments, DR′₃ is hydrogen or

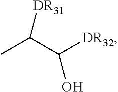

wherein DR₃₁ is H or a C₁₋₂ aliphatic that is optionally substituted with 1-3 of halo, —OH, or combinations thereof. DR₃₂ is -L-DR₃₃, wherein L is a bond, —CH₂—, —CH₂O—, —CH₂NHS(O)2-, —CH₂C(O)—, —CH₂NHC(O)—, or —CH₂NH—; and DR₃₃ is hydrogen, or C₁₋₂ aliphatic, cycloaliphatic, heterocycloaliphatic, or heteroaryl, each of which is optionally substituted with 1 of —OH, —NH₂, or —CN. For example, in one embodiment, DR₃₁ is hydrogen and DR₃₂ is C₁₋₂ aliphatic optionally substituted with —OH, —NH₂, or —CN.

In several embodiments, DR′₃ is independently selected from one of the following:

### —H, —CH₃, —CH₂CH₃, —C(O)CH₃, —CH₂CH₂OH, —C(O)OCH₃,

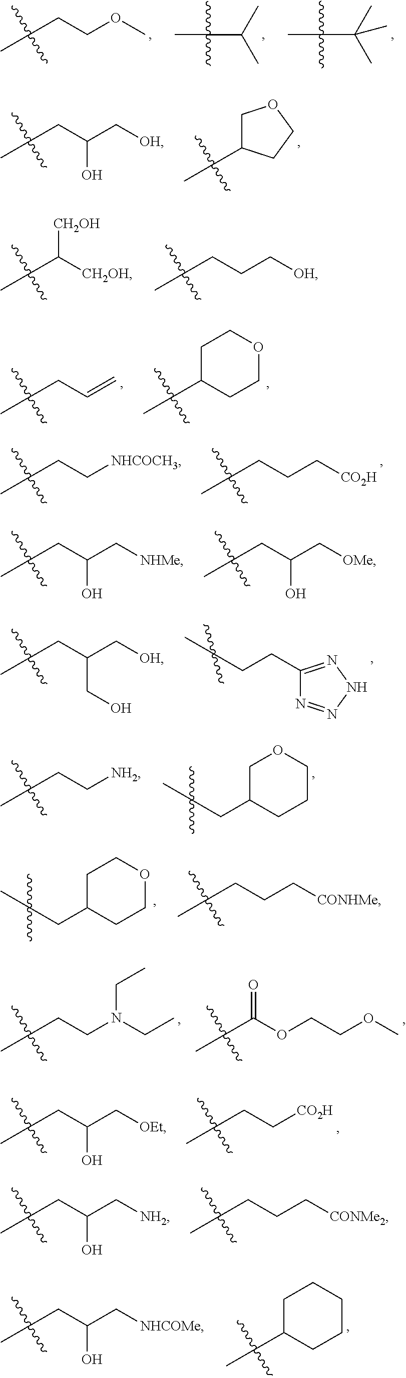

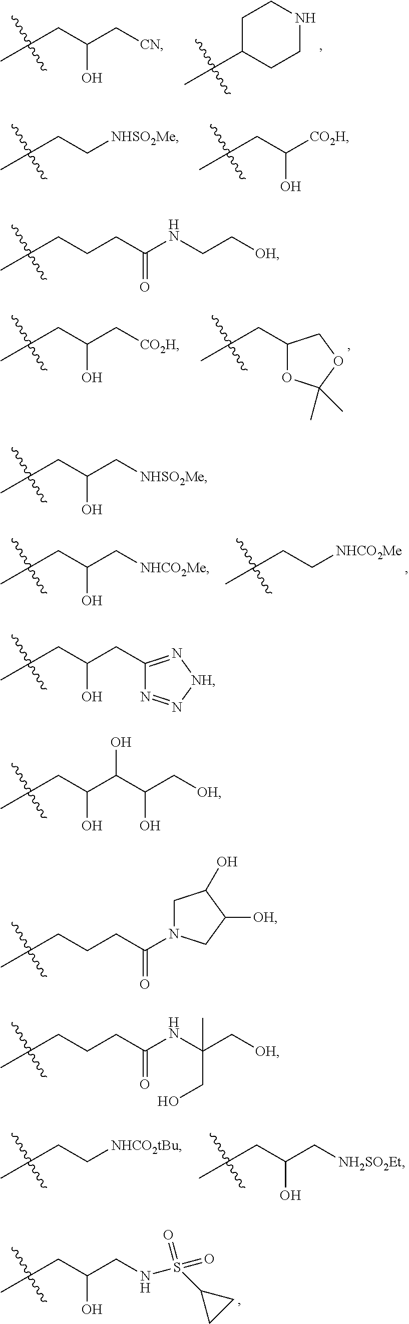

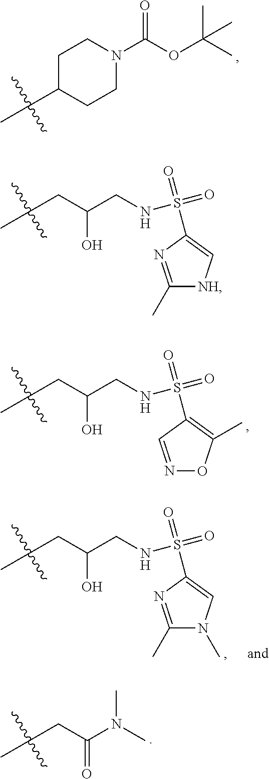

5. n Term

n is 1-3.

In several embodiments, n is 1. In other embodiments, n is 2. In still other embodiments, n is 3.

C. Exemplary Formula D Compounds 1-322 of the Present Invention

Exemplary Column D compounds (Of Formula D) 1-322 of the present invention include, but are not limited to those illustrated in Table II.D-1 below.

TABLE II.D-1
Exemplary compounds 1-322 of the present invention.

| | |
|---|---|
| 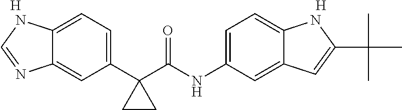 | 1 |
| 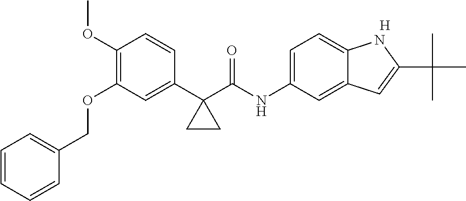 | 2 |
| 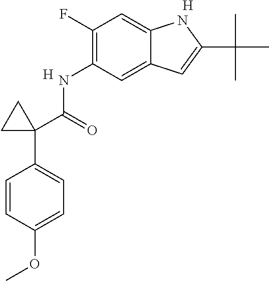 | 3 |
| 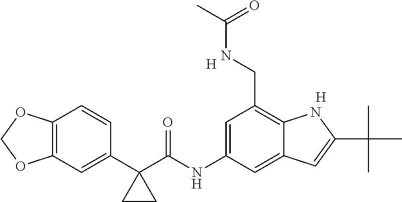 | 4 |
| 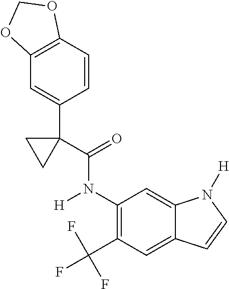 [SMILES: O=C(Nc1cc2[nH]ccc2cc1C(F)(F)F)C1(c2ccc3c(c2)OCO3)CC1] | 5 |
| 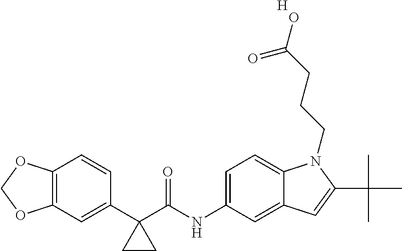 | 6 |
| 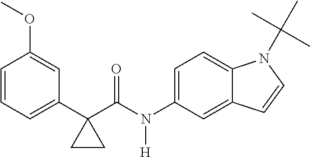 [SMILES: COc1cccc(C2(C(=O)Nc3ccc4c(ccn4C(C)(C)C)c3)CC2)c1] | 7 |
| 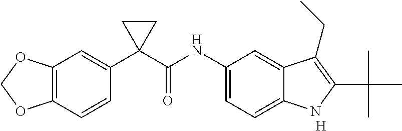 | 8 |
| 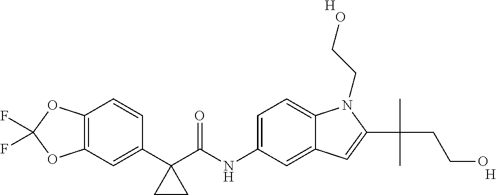 | 9 |
| 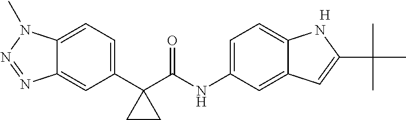 | 10 |
| 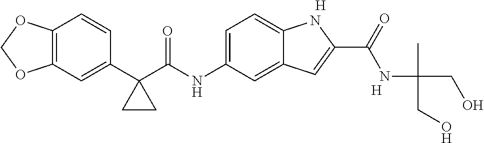 | 11 |
| 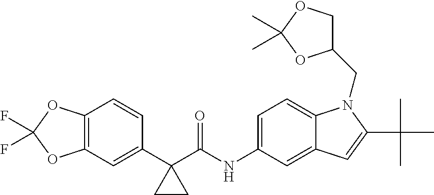 [SMILES: CC1(C)OCC(Cn2c(C(C)(C)C)cc3cc(NC(=O)C4(c5ccc6c(c5)OC(F)(F)O6)CC4)ccc32)O1] | 12 |
| 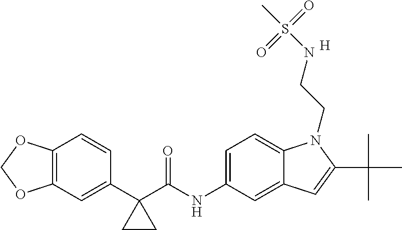 | 13 |
| 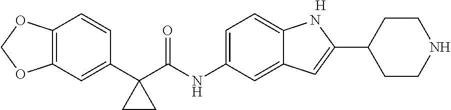 | 14 |
| 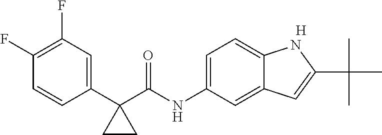 | 15 |
| 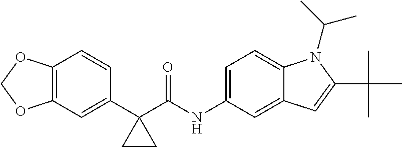 [SMILES: CC(C)n1c(C(C)(C)C)cc2cc(NC(=O)C3(c4ccc5c(c4)OCO5)CC3)ccc21] | 16 |
| 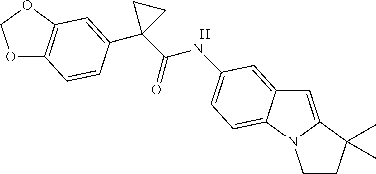 | 17 |
| 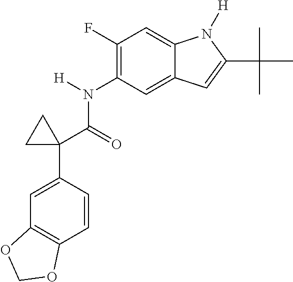 [SMILES: CC(C)(C)c1cc2cc(NC(=O)C3(c4ccc5c(c4)OCO5)CC3)c(F)cc2[nH]1] | 18 |
| 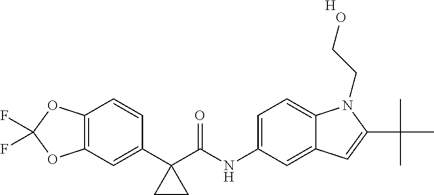 | 19 |
| 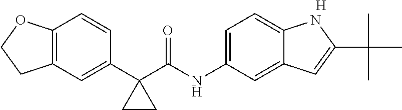 | 20 |
| 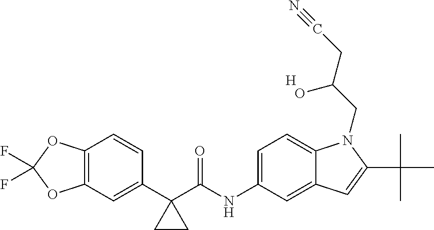 | 21 |
| 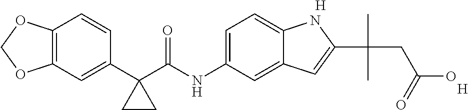 | 22 |
| 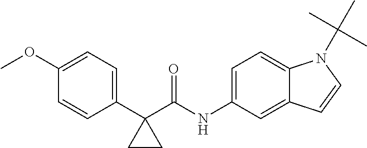 [SMILES: COc1ccc(C2(C(=O)Nc3ccc4c(ccn4C(C)(C)C)c3)CC2)cc1] | 23 |
| 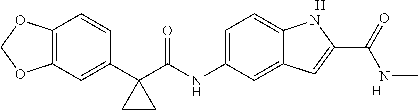 | 24 |
| 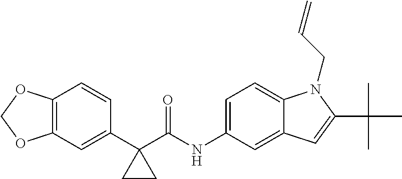 [SMILES: C=CCn1c(C(C)(C)C)cc2cc(NC(=O)C3(c4ccc5c(c4)OCO5)CC3)ccc21] | 25 |
| 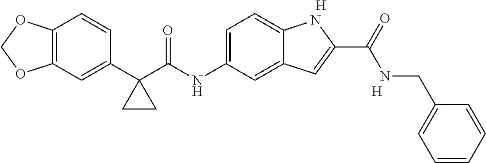 | 26 |
| 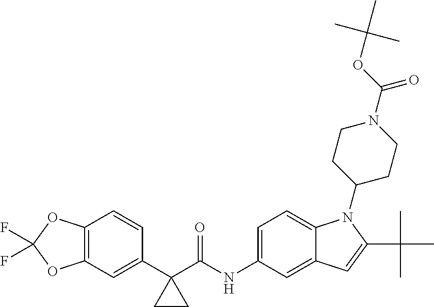 [SMILES: CC(C)(C)OC(=O)N1CCC(n2c(C(C)(C)C)cc3cc(NC(=O)C4(c5ccc6c(c5)OC(F)(F)O6)CC4)ccc32)CC1] | 27 |
| 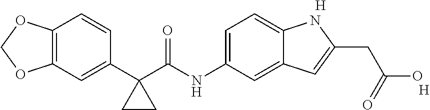 | 28 |
| 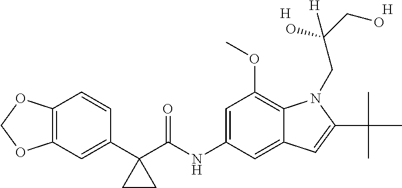 | 29 |
| 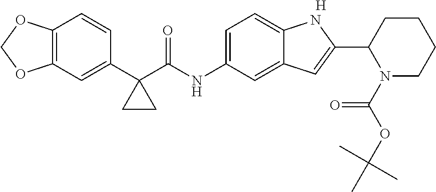 | 30 |
| 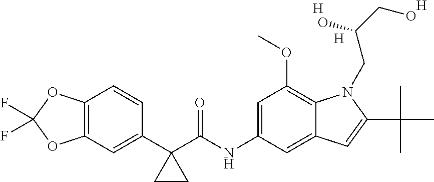 | 31 |
| 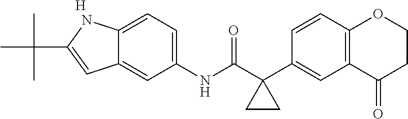 | 32 |
| 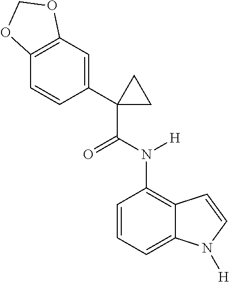 [SMILES: O=C(Nc1cccc2[nH]ccc12)C1(c2ccc3c(c2)OCO3)CC1] | 33 |
| 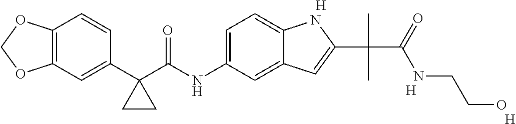 | 34 |
| 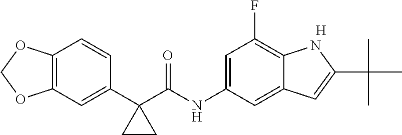 | 35 |
| 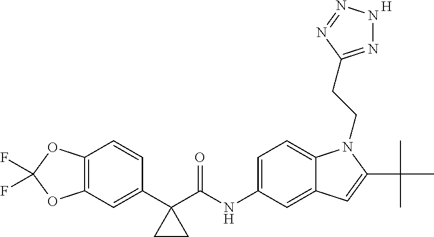 | 36 |
| 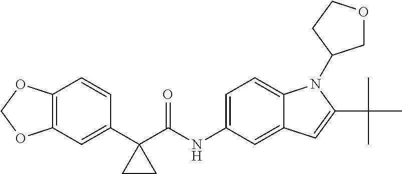 [SMILES: CC(C)(C)c1cc2cc(NC(=O)C3(c4ccc5c(c4)OCO5)CC3)ccc2n1C1CCOC1] | 37 |
| 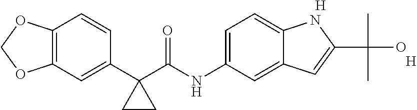 | 38 |
| 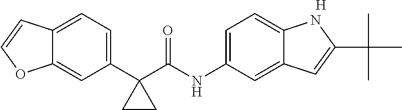 | 39 |
| 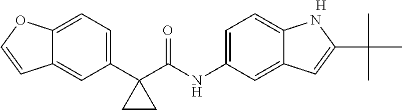 | 40 |
| 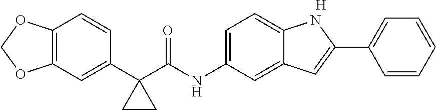 | 41 |
| 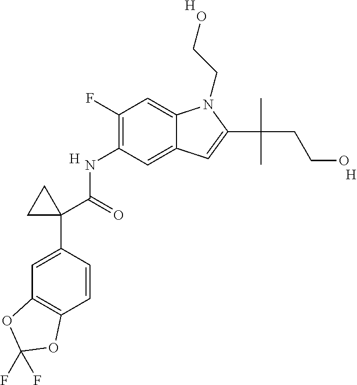 | 42 |
| 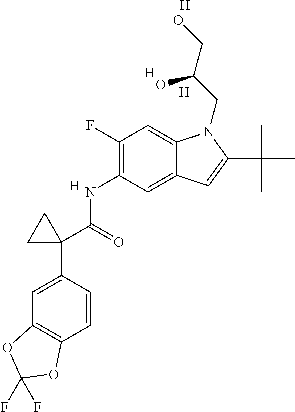 | 43 |
| 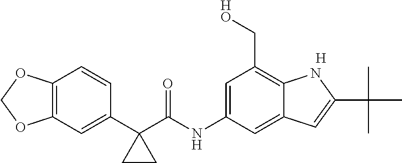 | 44 |
| 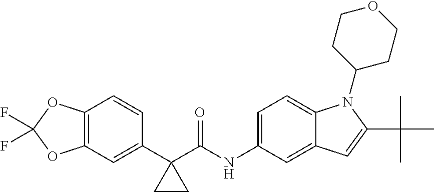 [SMILES: CC(C)(C)c1cc2cc(NC(=O)C3(c4ccc5c(c4)OC(F)(F)O5)CC3)ccc2n1C1CCOCC1] | 45 |
| 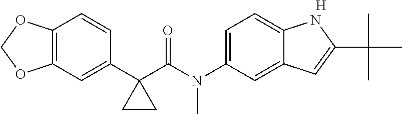 | 46 |
| 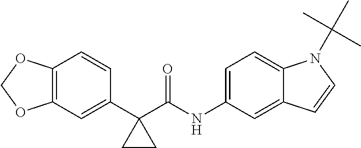 [SMILES: CC(C)(C)n1ccc2cc(NC(=O)C3(c4ccc5c(c4)OCO5)CC3)ccc21] | 47 |
| 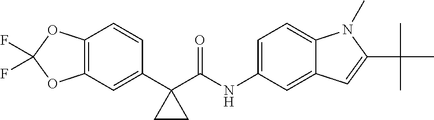 [SMILES: Cn1c(C(C)(C)C)cc2cc(NC(=O)C3(c4ccc5c(c4)OC(F)(F)O5)CC3)ccc21] | 48 |
| 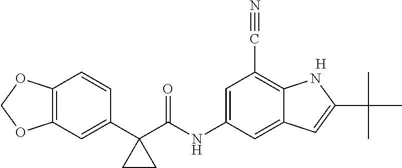 | 49 |
| 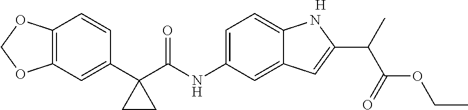 | 50 |
| 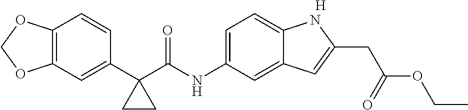 | 51 |
| 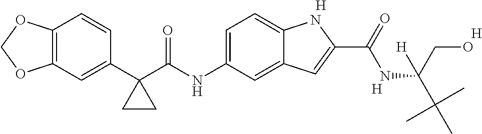 | 52 |
| 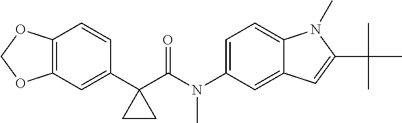 [SMILES: CN(C(=O)C1(c2ccc3c(c2)OCO3)CC1)c1ccc2c(c1)cc(C(C)(C)C)n2C] | 53 |
| 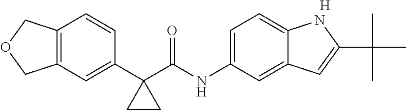 | 54 |
| 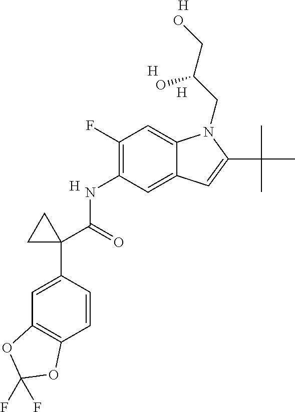 | 55 |
| 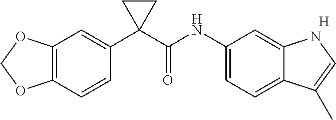 | 56 |
| 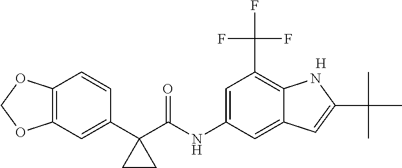 | 57 |
| 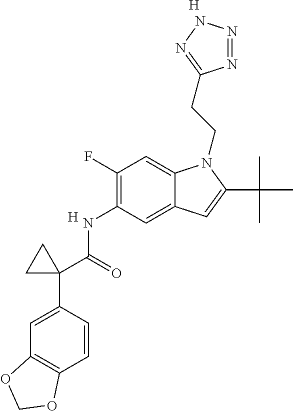 | 58 |
| 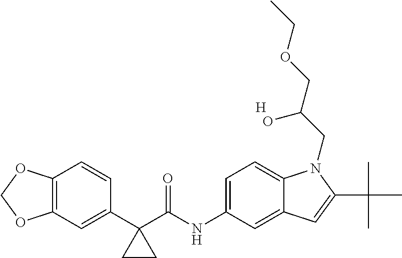 | 59 |
| 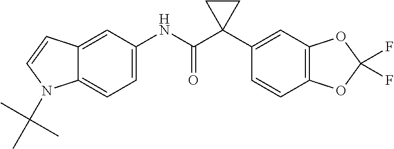 [SMILES: CC(C)(C)n1ccc2cc(NC(=O)C3(c4ccc5c(c4)OC(F)(F)O5)CC3)ccc21] | 60 |
| 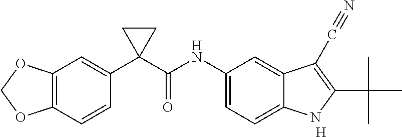 | 61 |
| 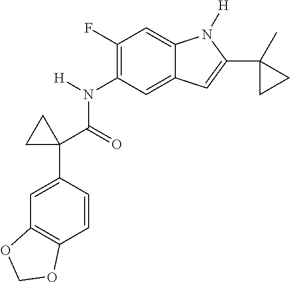 [SMILES: CC1(c2cc3cc(NC(=O)C4(c5ccc6c(c5)OCO6)CC4)c(F)cc3[nH]2)CC1] | 62 |
| 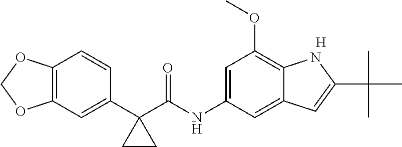 | 63 |
| 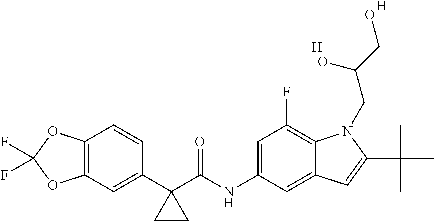 | 64 |
| 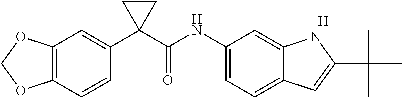 | 65 |
| 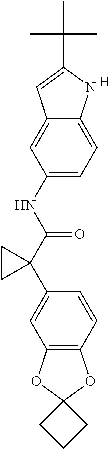 | 66 |
| 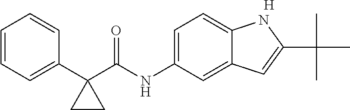 | 67 |
| 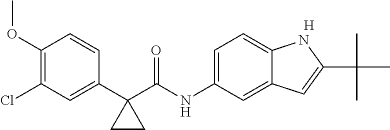 | 68 |
| 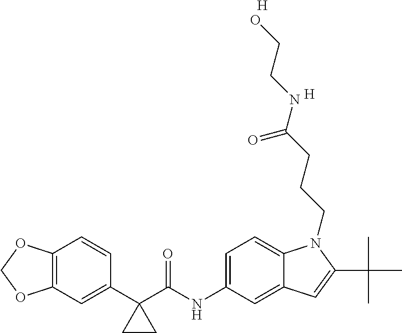 | 69 |
| 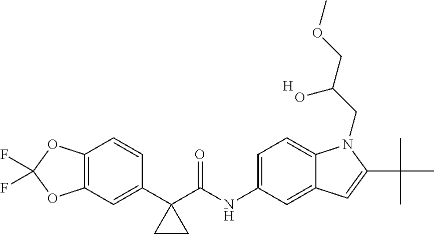 | 70 |
| 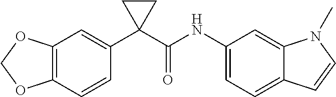 [SMILES: Cn1ccc2ccc(NC(=O)C3(c4ccc5c(c4)OCO5)CC3)cc21] | 71 |
| 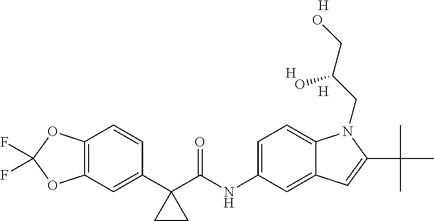 | 72 |
| 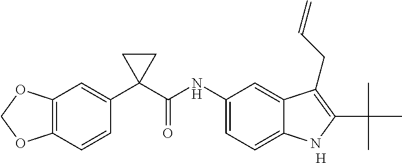 | 73 |
| 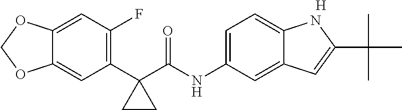 | 74 |
| 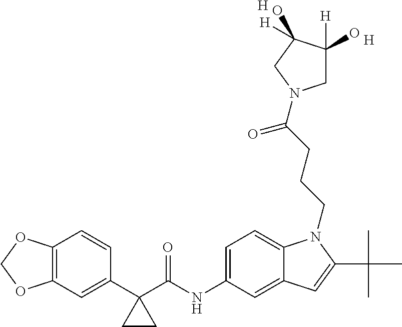 | 75 |
| 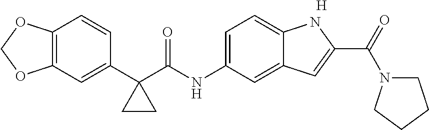 | 76 |
| 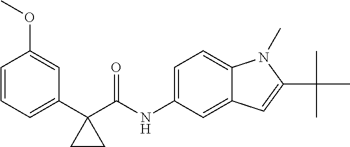 [SMILES: COc1cccc(C2(C(=O)Nc3ccc4c(c3)cc(C(C)(C)C)n4C)CC2)c1] | 77 |
| 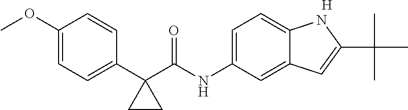 | 78 |
| 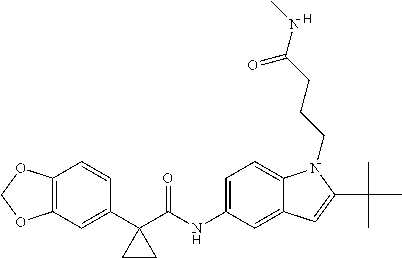 | 79 |
| 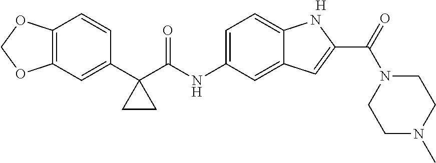 | 80 |
| 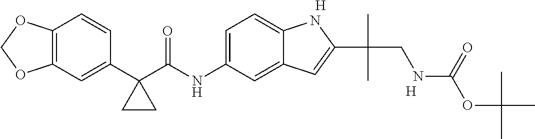 | 81 |
| 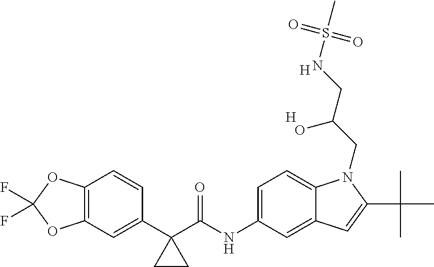 | 82 |
| 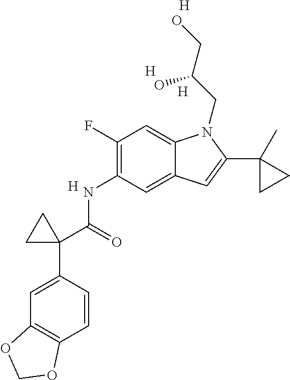 | 83 |
| 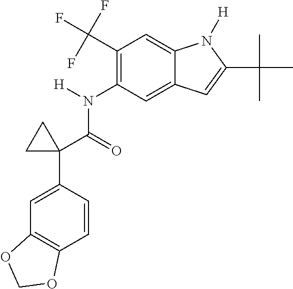 [SMILES: CC(C)(C)c1cc2cc(NC(=O)C3(c4ccc5c(c4)OCO5)CC3)c(C(F)(F)F)cc2[nH]1] | 84 |
| 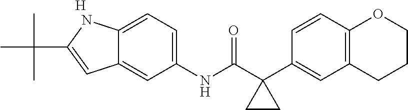 | 85 |
| 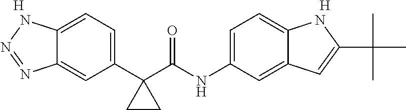 | 86 |
| 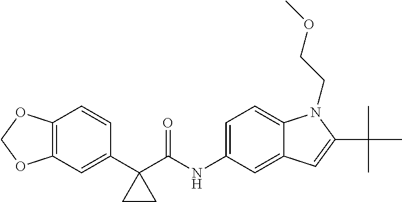 [SMILES: COCCn1c(C(C)(C)C)cc2cc(NC(=O)C3(c4ccc5c(c4)OCO5)CC3)ccc21] | 87 |
| 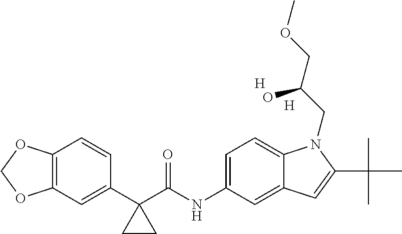 | 88 |
| 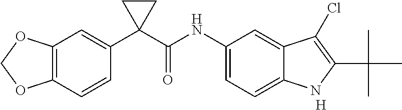 | 89 |
| 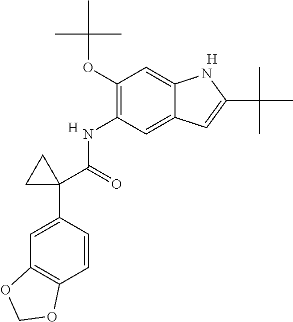 | 90 |
| 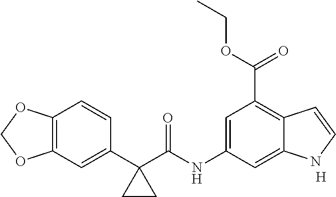 | 91 |
| 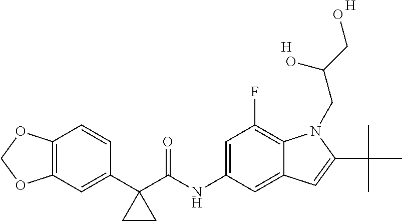 | 92 |
| 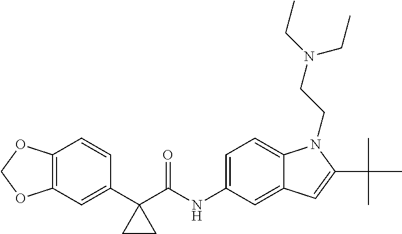 [SMILES: CCN(CC)CCn1c(C(C)(C)C)cc2cc(NC(=O)C3(c4ccc5c(c4)OCO5)CC3)ccc21] | 93 |
| 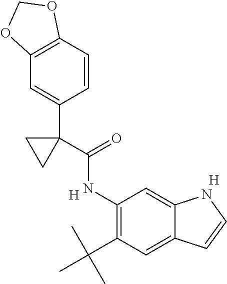 | 94 |
| 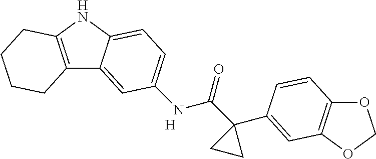 | 95 |
| 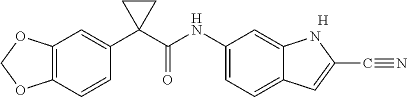 | 96 |
| 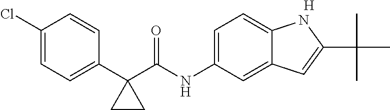 | 97 |
| 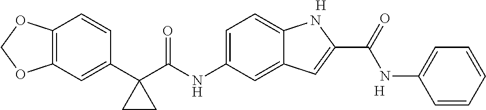 | 98 |
| 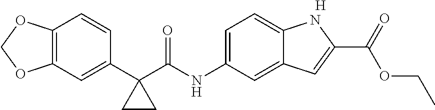 | 99 |
| 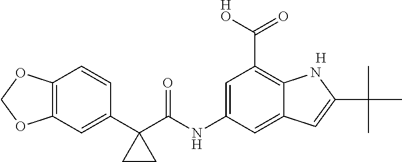 | 100 |
| 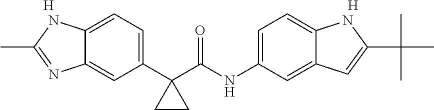 | 101 |
| 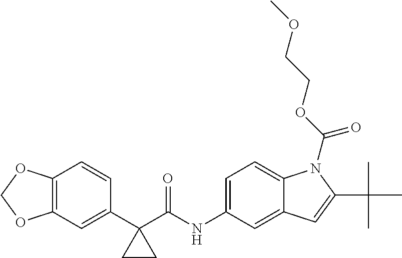 [SMILES: COCCOC(=O)n1c(C(C)(C)C)cc2cc(NC(=O)C3(c4ccc5c(c4)OCO5)CC3)ccc21] | 102 |
| 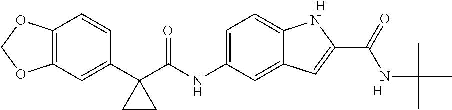 | 103 |
| 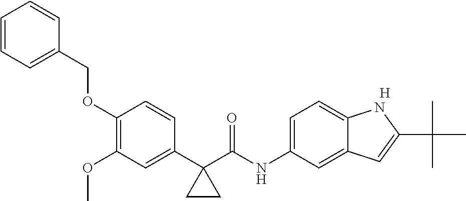 | 104 |
| 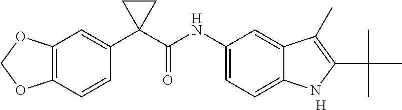 | 105 |
| 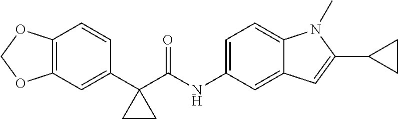 [SMILES: Cn1c(C2CC2)cc2cc(NC(=O)C3(c4ccc5c(c4)OCO5)CC3)ccc21] | 106 |
| 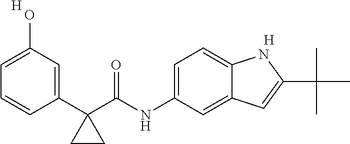 | 107 |
| 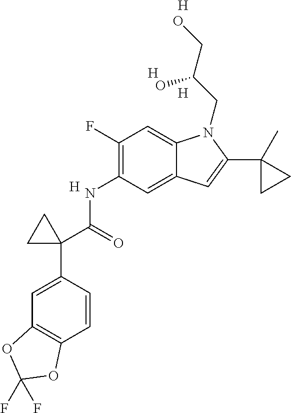 | 108 |
| 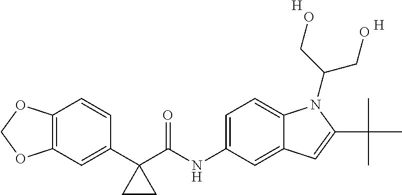 | 109 |
| 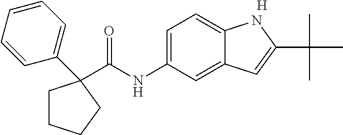 | 110 |
| 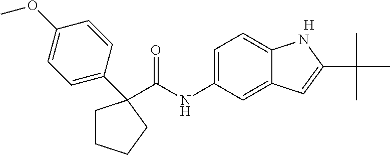 | 111 |
| 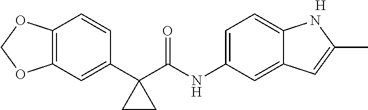 | 112 |
| 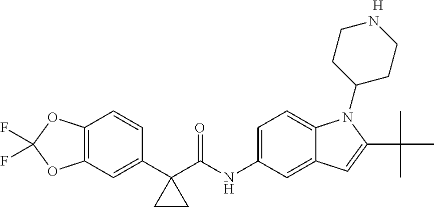 | 113 |
| 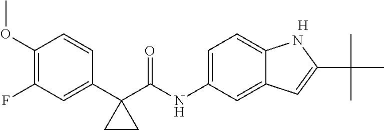 | 114 |
| 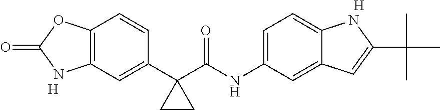 | 115 |
| 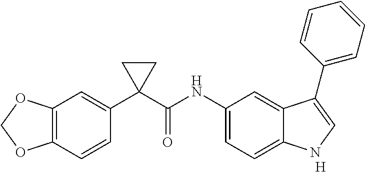 | 116 |
| 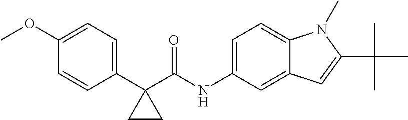 [SMILES: COc1ccc(C2(C(=O)Nc3ccc4c(c3)cc(C(C)(C)C)n4C)CC2)cc1] | 117 |
| 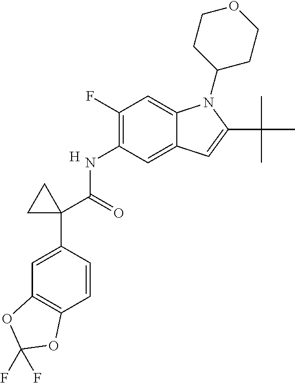 | 118 |
| 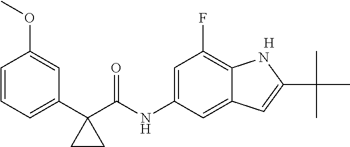 | 119 |
| 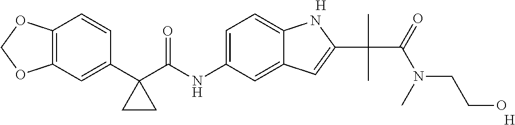 | 120 |
| 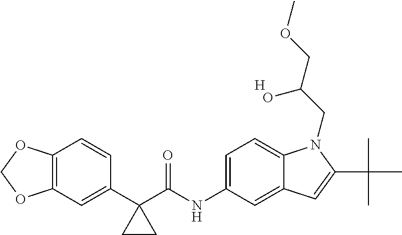 | 121 |
| 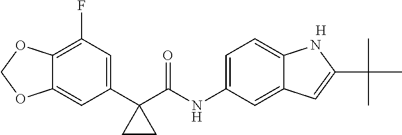 | 122 |
| 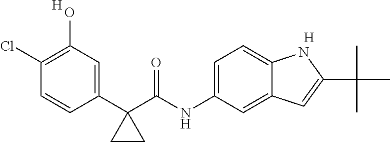 | 123 |
| 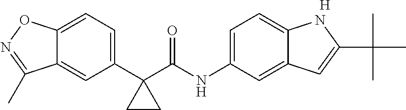 | 124 |
| 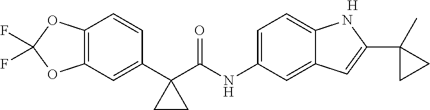 | 125 |
| 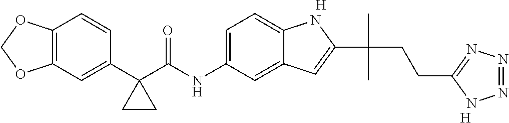 | 126 |
| 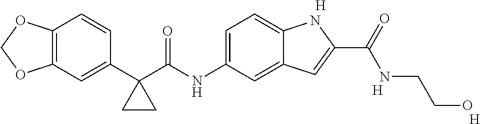 | 127 |
| 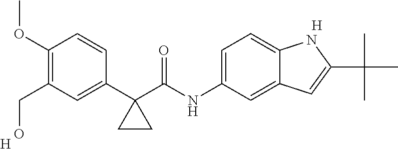 | 128 |
| 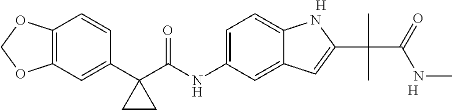 | 129 |
| 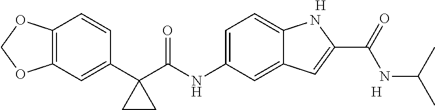 | 130 |
| 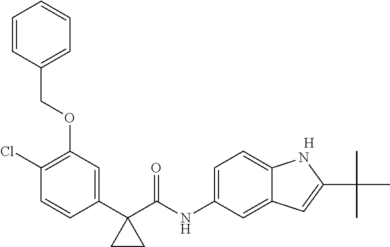 | 131 |
| 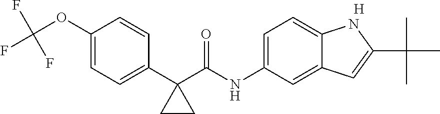 | 132 |
| 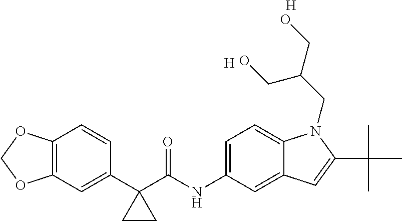 | 133 |
| 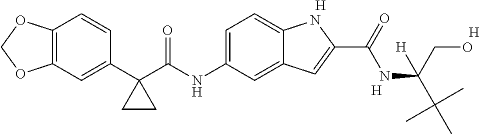 | 134 |
| 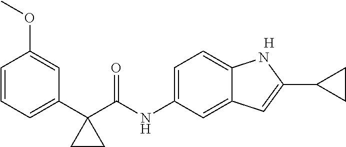 | 135 |
| 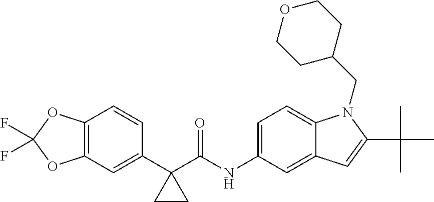 [SMILES: CC(C)(C)c1cc2cc(NC(=O)C3(c4ccc5c(c4)OC(F)(F)O5)CC3)ccc2n1CC1CCOCC1] | 136 |
| 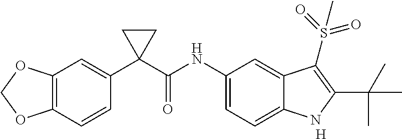 | 137 |
| 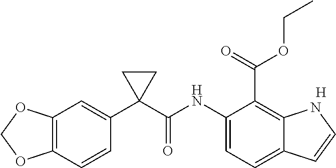 | 138 |
| 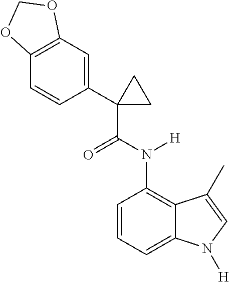 [SMILES: Cc1c[nH]c2cccc(NC(=O)C3(c4ccc5c(c4)OCO5)CC3)c12] | 139 |
| 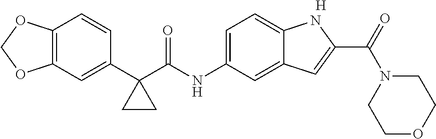 | 140 |
| 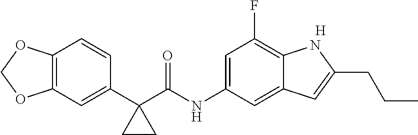 | 141 |
| 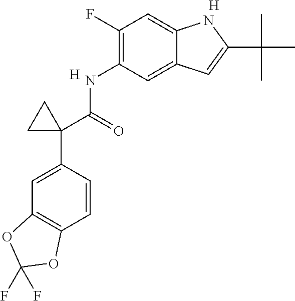 | 142 |
| 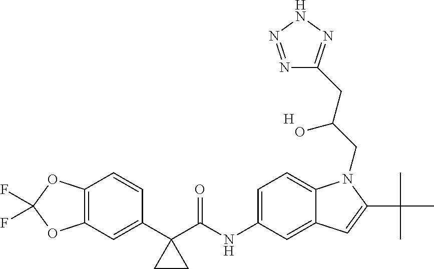 | 143 |
| 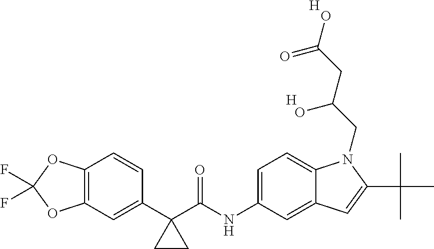 | 144 |
| 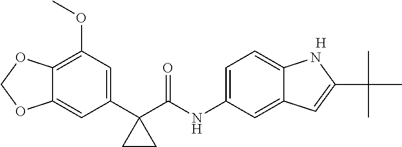 | 145 |
| 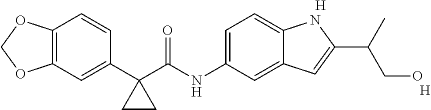 | 146 |
| 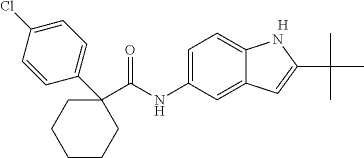 | 147 |
| 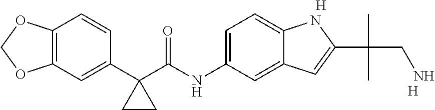 | 148 |
| 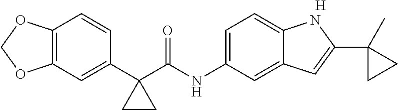 | 149 |
| 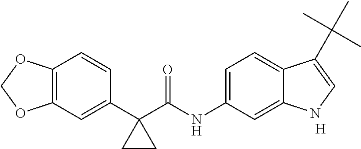 | 150 |
| 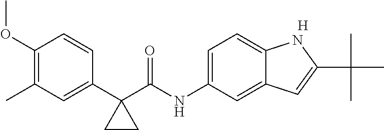 | 151 |
| 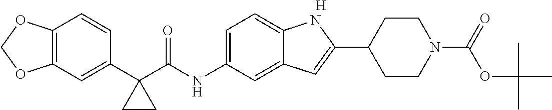 | 152 |
| 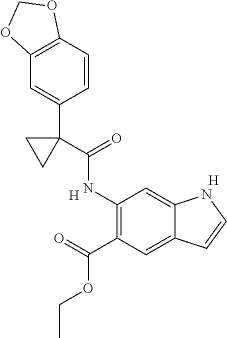 | 153 |
| 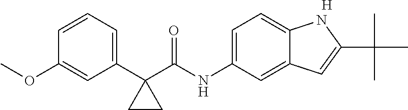 | 154 |
| 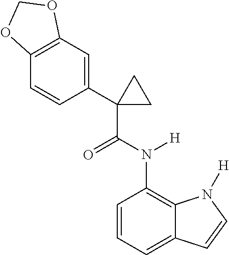 [SMILES: O=C(Nc1cccc2cc[nH]c12)C1(c2ccc3c(c2)OCO3)CC1] | 155 |
| 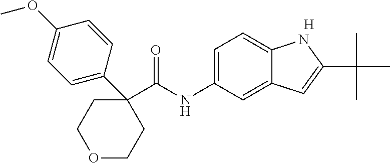 | 156 |
| 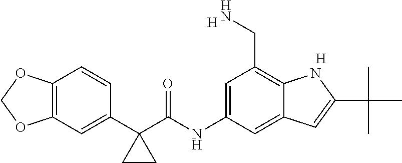 | 157 |
| 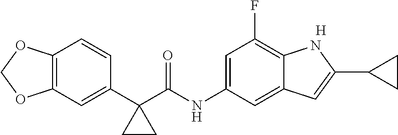 | 158 |
| 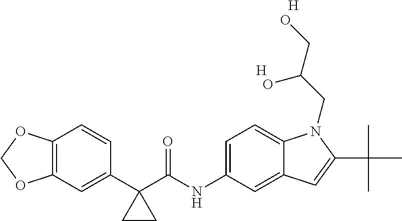 | 159 |
| 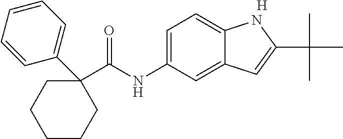 | 160 |
| 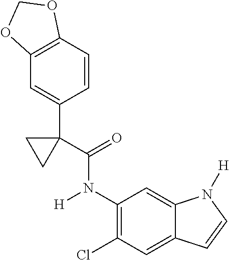 [SMILES: O=C(Nc1cc2[nH]ccc2cc1Cl)C1(c2ccc3c(c2)OCO3)CC1] | 161 |
| 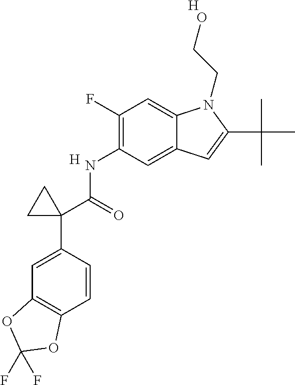 | 162 |
| 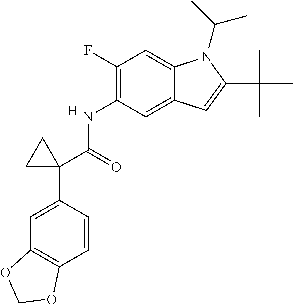 | 163 |
| 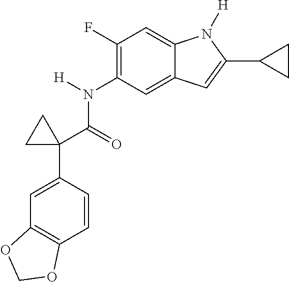 [SMILES: O=C(Nc1cc2cc(C3CC3)[nH]c2cc1F)C1(c2ccc3c(c2)OCO3)CC1] | 164 |
| 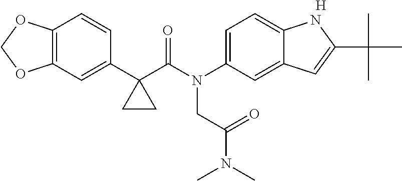 | 165 |
| 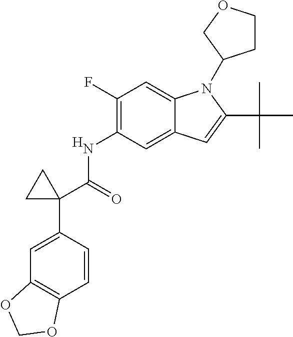 | 166 |
| 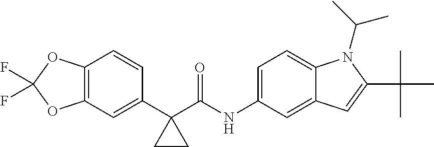 [SMILES: CC(C)n1c(C(C)(C)C)cc2cc(NC(=O)C3(c4ccc5c(c4)OC(F)(F)O5)CC3)ccc21] | 167 |
| 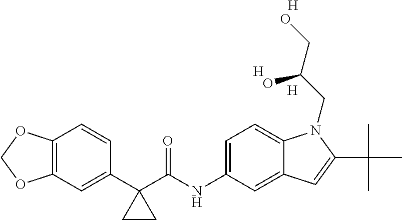 | 168 |
| 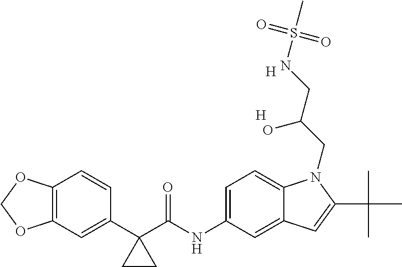 | 169 |
| 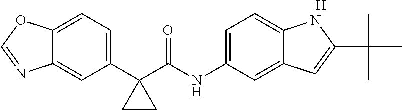 | 170 |
| 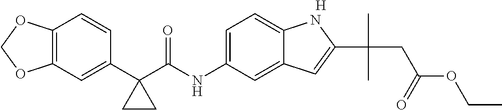 | 171 |
| 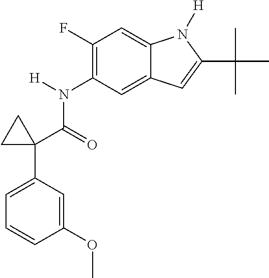 [SMILES: COc1cccc(C2(C(=O)Nc3cc4cc(C(C)(C)C)[nH]c4cc3F)CC2)c1] | 172 |
| 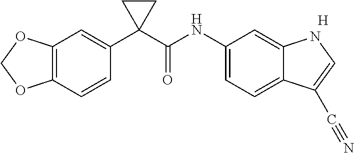 | 173 |
| 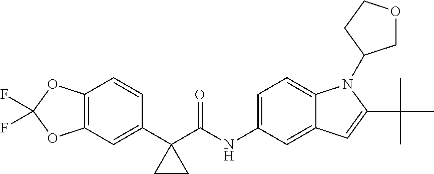 [SMILES: CC(C)(C)c1cc2cc(NC(=O)C3(c4ccc5c(c4)OC(F)(F)O5)CC3)ccc2n1C1CCOC1] | 174 |
| 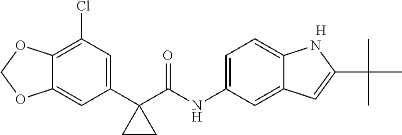 | 175 |
| 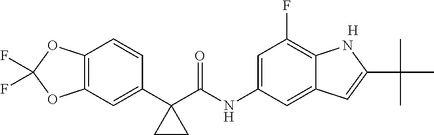 | 176 |
| 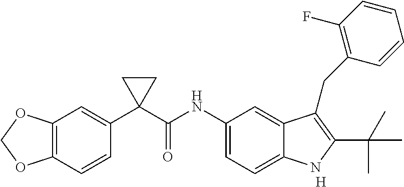 | 177 |
| 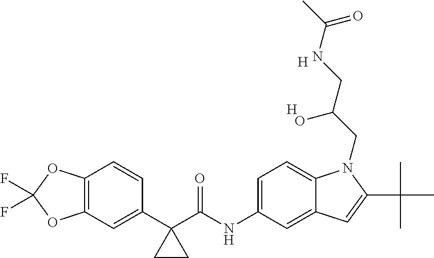 | 178 |
| 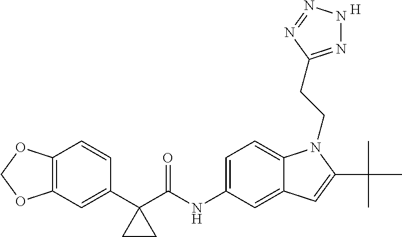 | 179 |
| 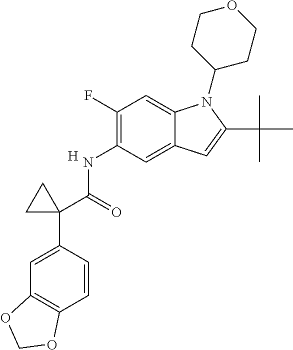 | 180 |
| 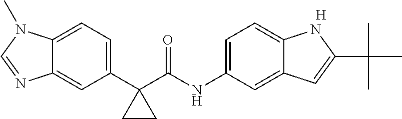 | 181 |
| 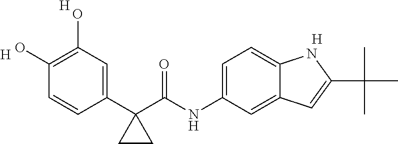 | 182 |
| 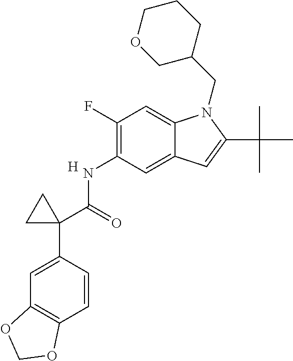 | 183 |
| 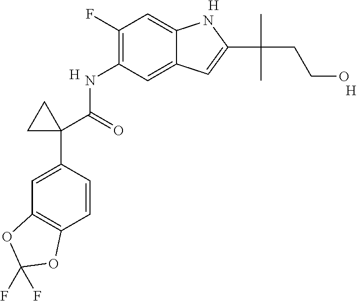 | 184 |
| 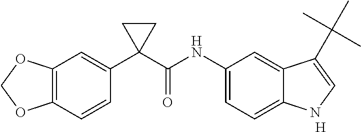 | 185 |
| 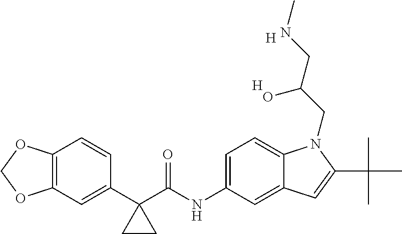 | 186 |
| 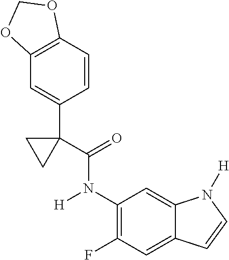 [SMILES: O=C(Nc1cc2[nH]ccc2cc1F)C1(c2ccc3c(c2)OCO3)CC1] | 187 |
| 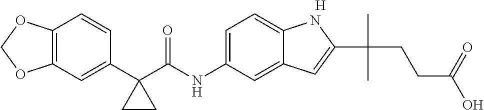 | 188 |
| 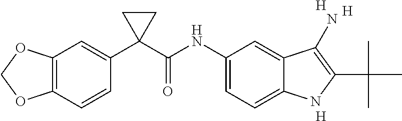 | 189 |
| 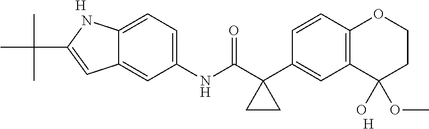 | 190 |
| 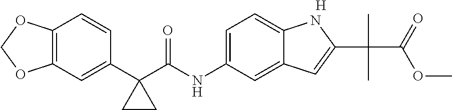 | 191 |
| 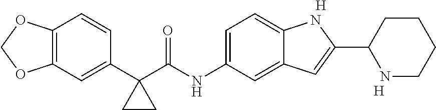 | 192 |
| 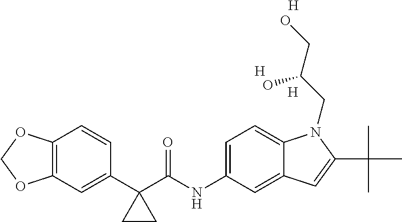 | 193 |
| 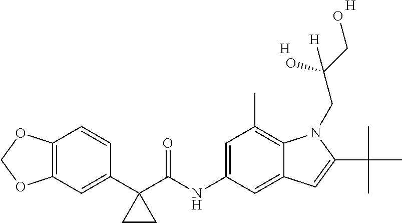 | 194 |
| 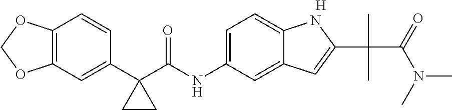 | 195 |
| 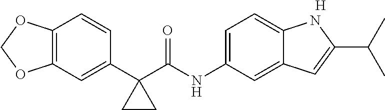 | 196 |
| 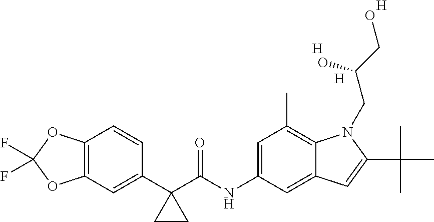 | 197 |
| 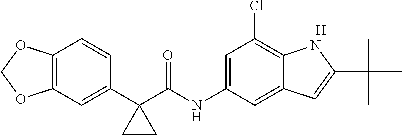 | 198 |
| 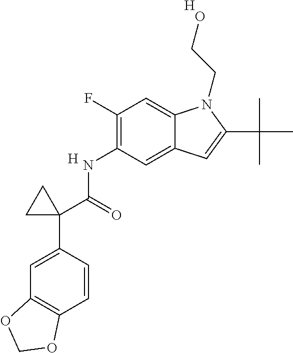 | 199 |
| 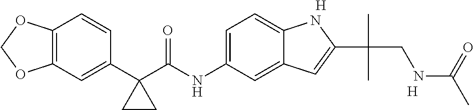 | 200 |
| 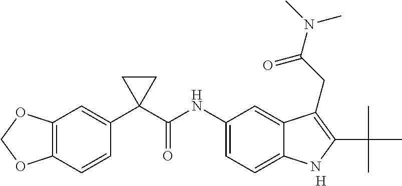 | 201 |
| 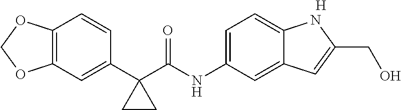 | 202 |
| 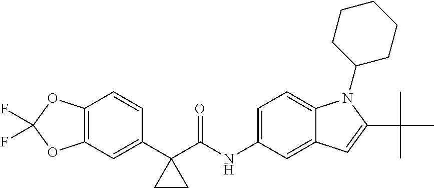 [SMILES: CC(C)(C)c1cc2cc(NC(=O)C3(c4ccc5c(c4)OC(F)(F)O5)CC3)ccc2n1C1CCCCC1] | 203 |
| 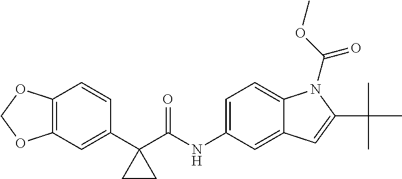 [SMILES: COC(=O)n1c(C(C)(C)C)cc2cc(NC(=O)C3(c4ccc5c(c4)OCO5)CC3)ccc21] | 204 |
| 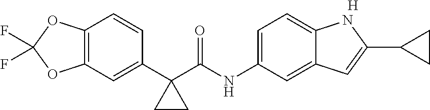 | 205 |
| 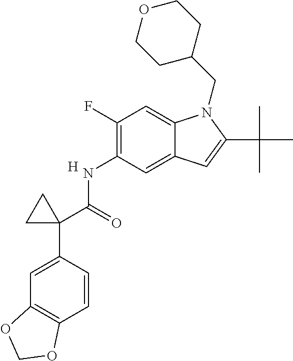 | 206 |
| 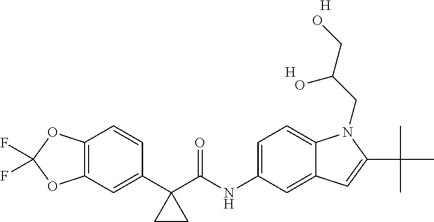 | 207 |
| 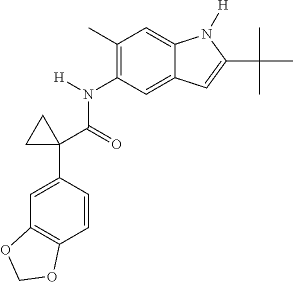 [SMILES: Cc1cc2[nH]c(C(C)(C)C)cc2cc1NC(=O)C1(c2ccc3c(c2)OCO3)CC1] | 208 |
| 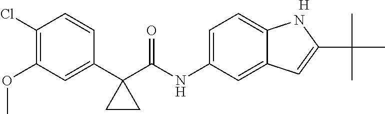 | 209 |
| 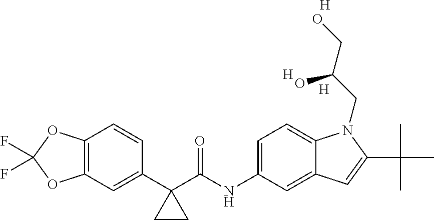 | 210 |
| 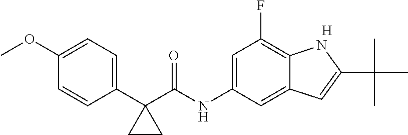 | 211 |
| 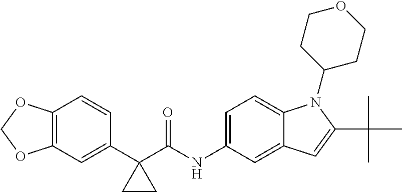 [SMILES: CC(C)(C)c1cc2cc(NC(=O)C3(c4ccc5c(c4)OCO5)CC3)ccc2n1C1CCOCC1] | 212 |
| 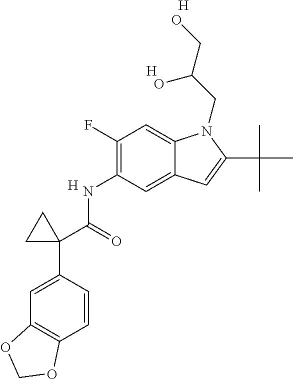 | 213 |
| 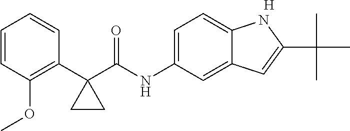 | 214 |
| 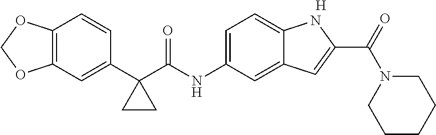 | 215 |
| 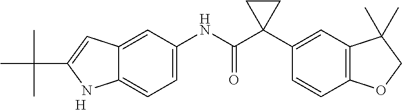 | 216 |
| 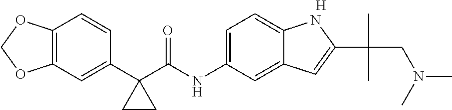 | 217 |
| 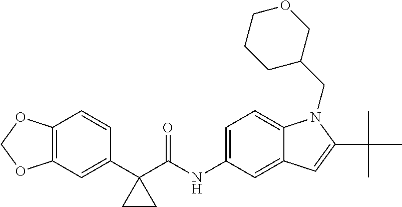 [SMILES: CC(C)(C)c1cc2cc(NC(=O)C3(c4ccc5c(c4)OCO5)CC3)ccc2n1CC1CCCOC1] | 218 |
| 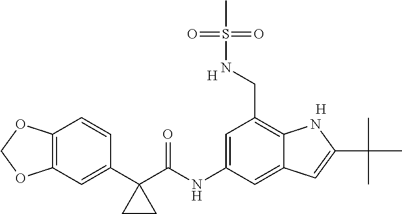 | 219 |
| 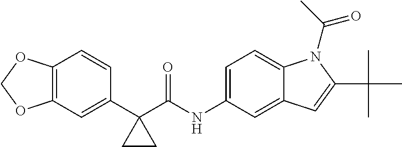 [SMILES: CC(=O)n1c(C(C)(C)C)cc2cc(NC(=O)C3(c4ccc5c(c4)OCO5)CC3)ccc21] | 220 |
| 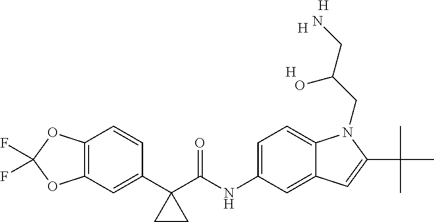 | 221 |
| 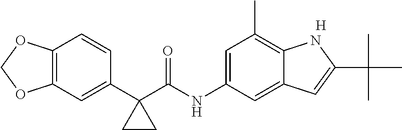 | 222 |
| 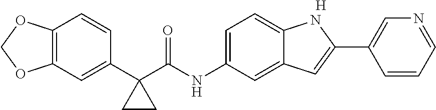 | 223 |
| 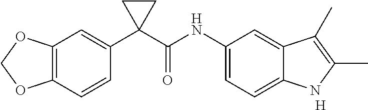 | 224 |
| 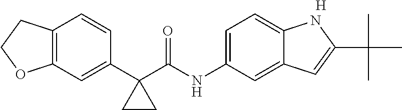 | 225 |
| 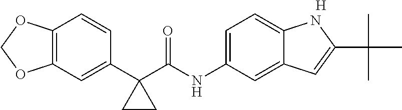 | 226 |
| 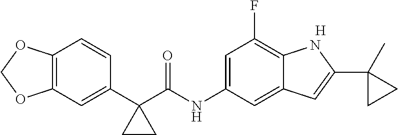 | 227 |
| 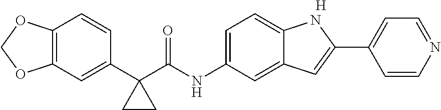 | 228 |
| 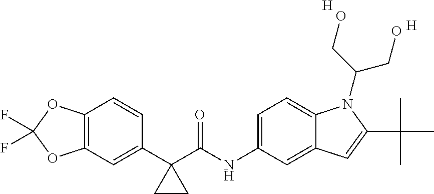 | 229 |
| 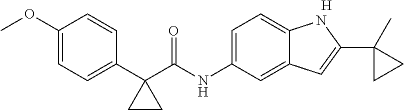 | 230 |
| 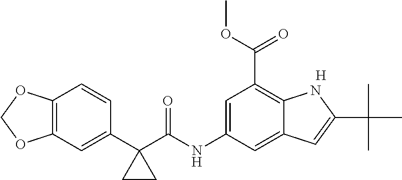 | 231 |
| 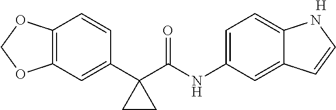 | 232 |
| 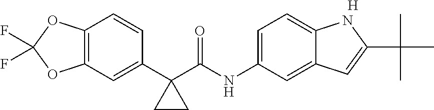 | 233 |
| 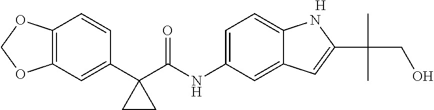 | 234 |
| 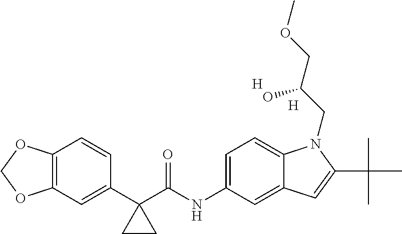 | 235 |
| 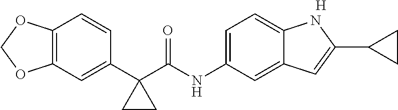 | 236 |
| 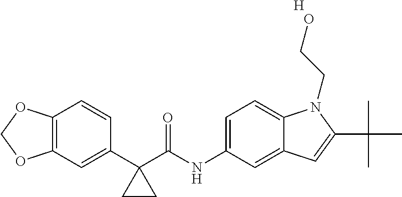 | 237 |
| 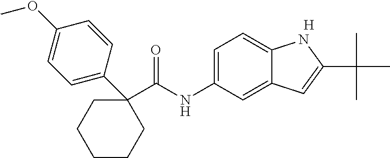 | 238 |
| 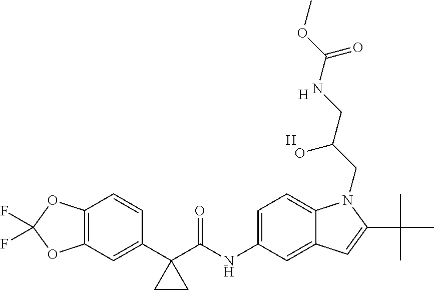 | 239 |
| 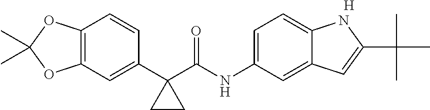 | 240 |
| 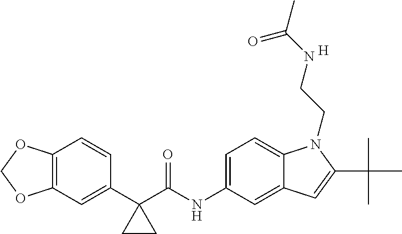 | 241 |
| 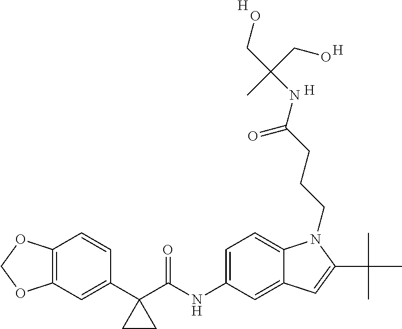 | 242 |
| 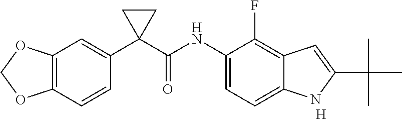 | 243 |
| 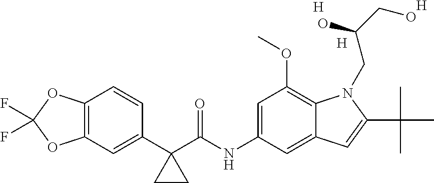 | 244 |
| 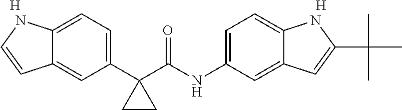 | 245 |
| 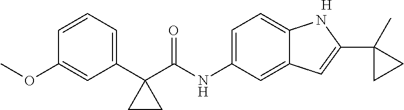 | 246 |
| 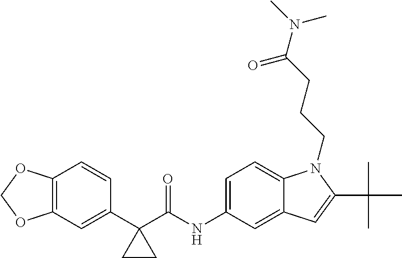 [SMILES: CN(C)C(=O)CCCn1c(C(C)(C)C)cc2cc(NC(=O)C3(c4ccc5c(c4)OCO5)CC3)ccc21] | 247 |
| 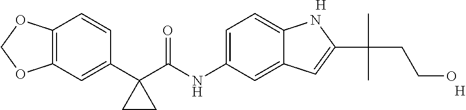 | 248 |
| 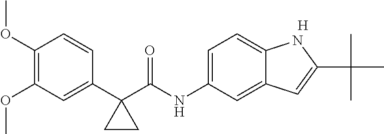 | 249 |
| 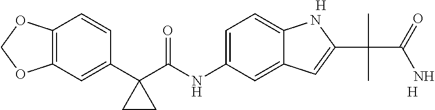 | 250 |
| 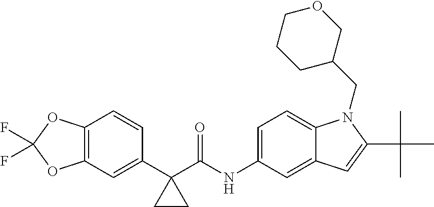 [SMILES: CC(C)(C)c1cc2cc(NC(=O)C3(c4ccc5c(c4)OC(F)(F)O5)CC3)ccc2n1CC1CCCOC1] | 251 |
| 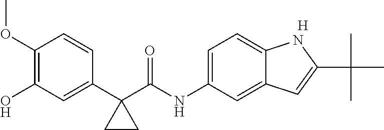 | 252 |
| 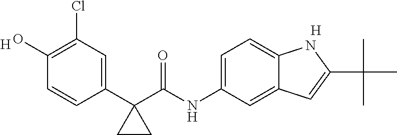 | 253 |
| 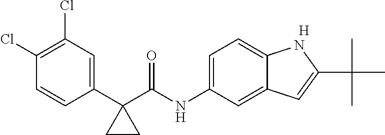 | 254 |
| 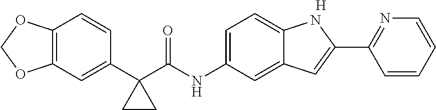 | 255 |
| 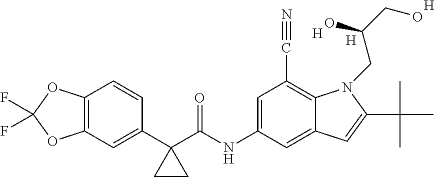 | 256 |
| 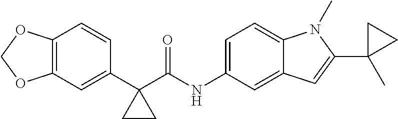 [SMILES: Cn1c(C2(C)CC2)cc2cc(NC(=O)C3(c4ccc5c(c4)OCO5)CC3)ccc21] | 257 |
| 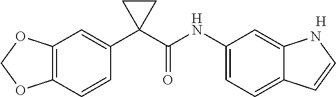 | 258 |
| 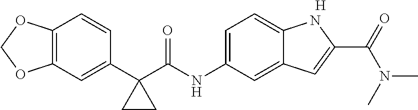 | 259 |
| 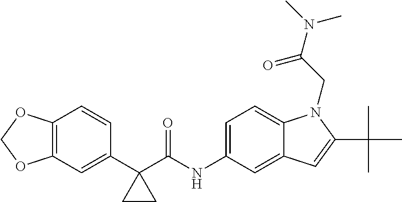 [SMILES: CN(C)C(=O)Cn1c(C(C)(C)C)cc2cc(NC(=O)C3(c4ccc5c(c4)OCO5)CC3)ccc21] | 260 |
| 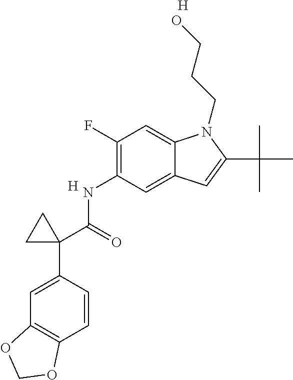 | 261 |
| 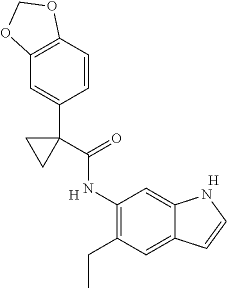 | 262 |
| 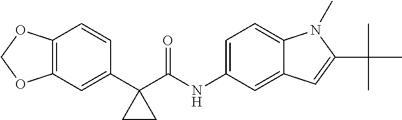 [SMILES: Cn1c(C(C)(C)C)cc2cc(NC(=O)C3(c4ccc5c(c4)OCO5)CC3)ccc21] | 263 |
| 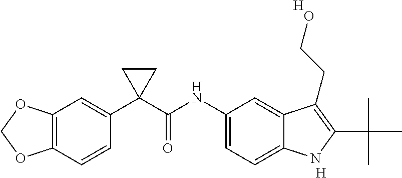 | 264 |
| 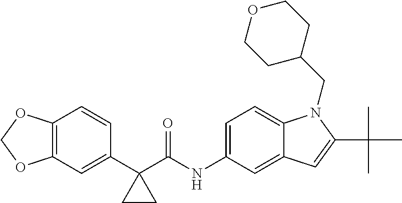 [SMILES: CC(C)(C)c1cc2cc(NC(=O)C3(c4ccc5c(c4)OCO5)CC3)ccc2n1CC1CCOCC1] | 265 |
| 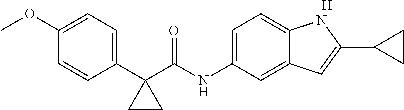 | 266 |
| 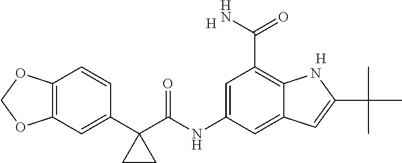 | 267 |
| 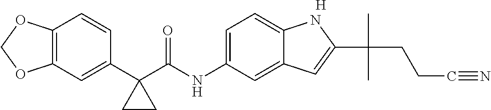 | 268 |
| 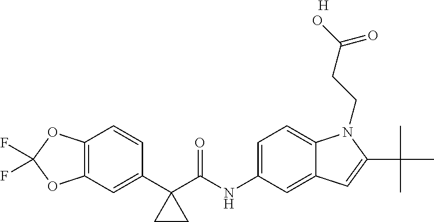 | 269 |
| 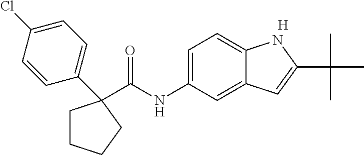 | 270 |
| 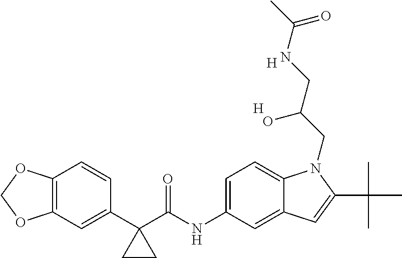 | 271 |
| 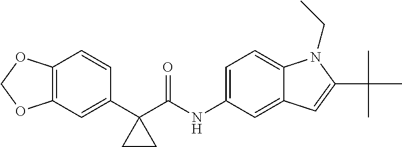 [SMILES: CCn1c(C(C)(C)C)cc2cc(NC(=O)C3(c4ccc5c(c4)OCO5)CC3)ccc21] | 272 |
| 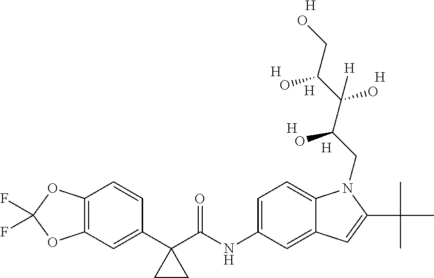 | 273 |
| 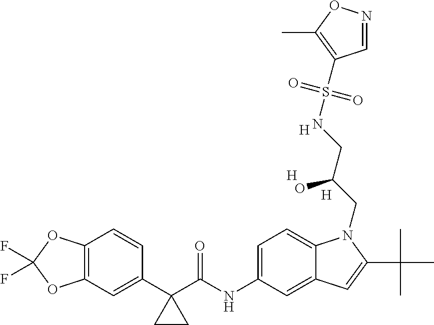 | 274 |
| 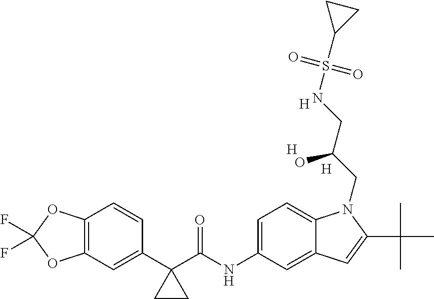 | 275 |
| 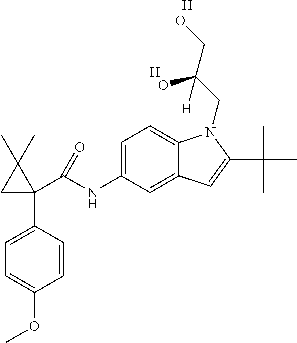 | 276 |
| 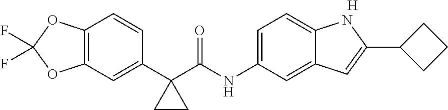 | 277 |
| 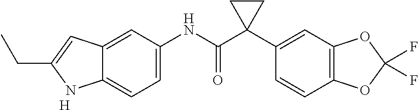 | 278 |
| 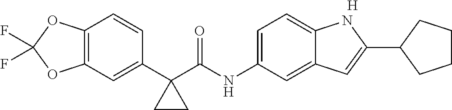 | 279 |
| 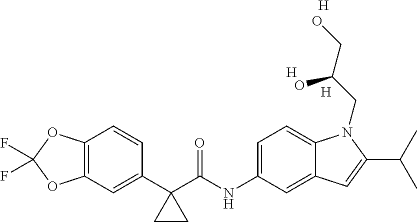 | 280 |
| 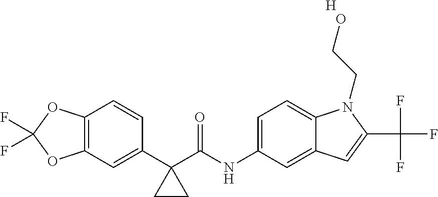 | 281 |
| 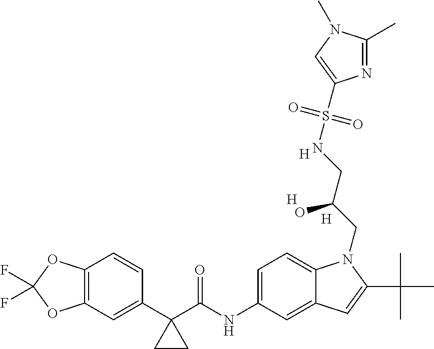 | 282 |
| 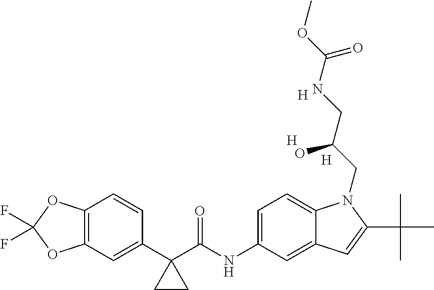 | 283 |
| 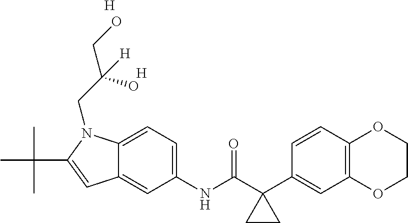 | 284 |
| 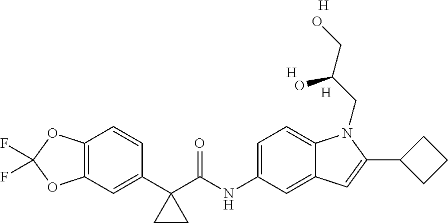 | 285 |
| 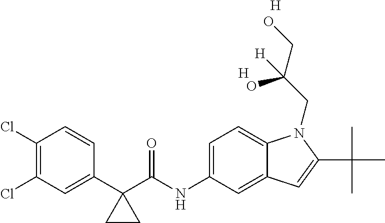 | 286 |
| 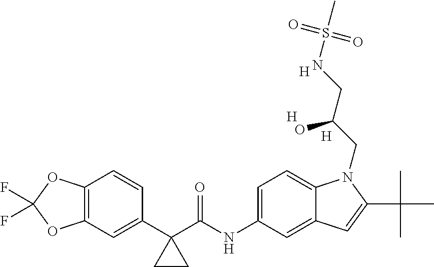 | 287 |
| 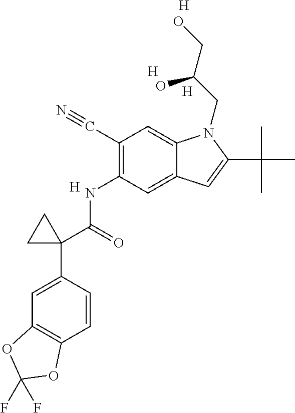 | 288 |
| 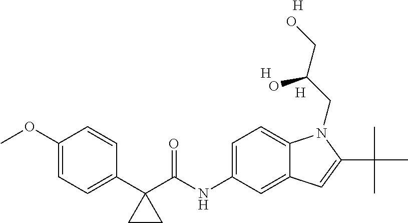 | 289 |
| 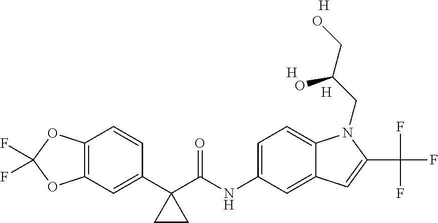 | 290 |
| 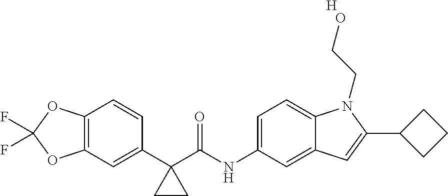 | 291 |
| 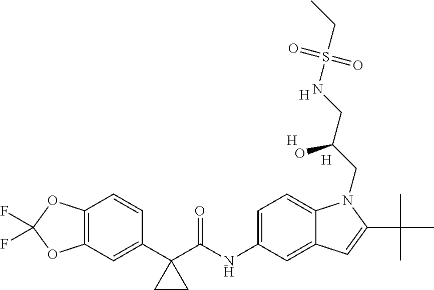 | 292 |
| 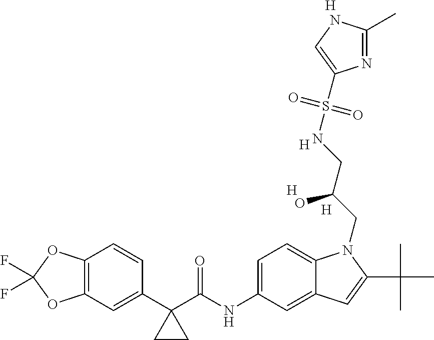 | 293 |
| 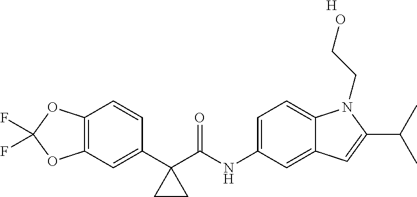 | 294 |
| 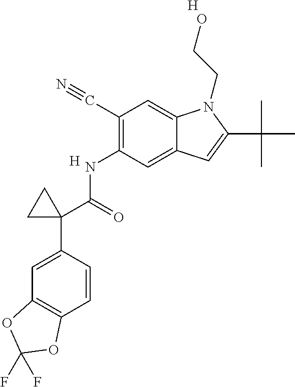 | 295 |
| 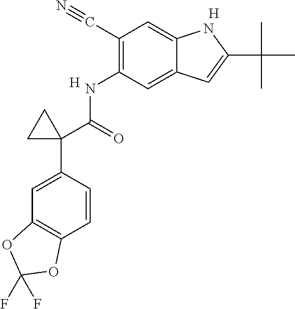 | 296 |
| 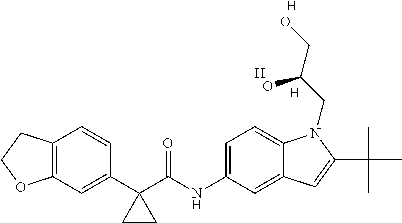 | 297 |
| 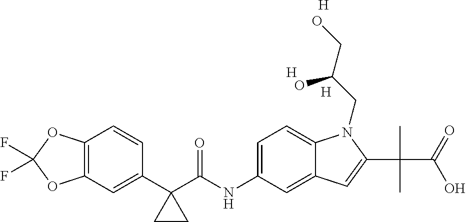 | 298 |
| 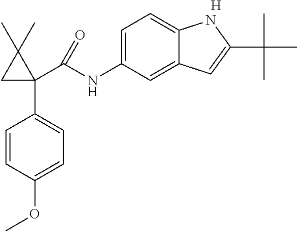 | 299 |
| 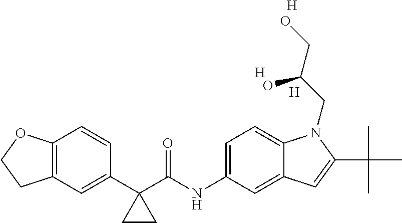 | 300 |
| 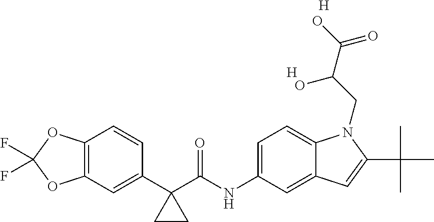 | 301 |
| 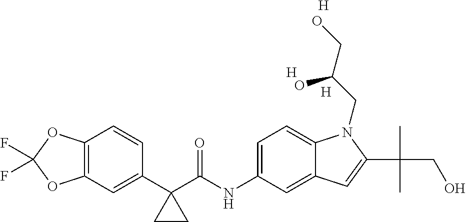 | 302 |
| 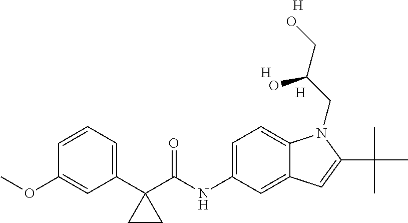 | 303 |
| 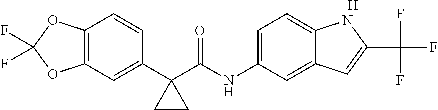 | 304 |
| 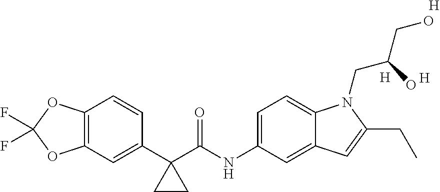 | 305 |
| 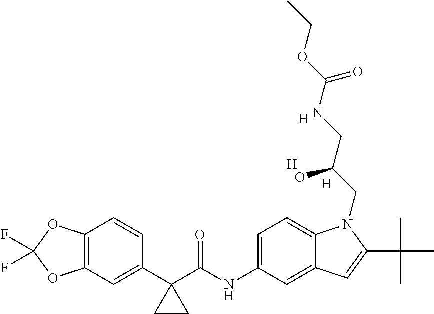 | 306 |
| 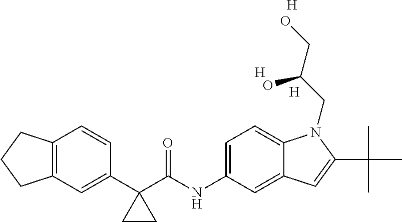 | 307 |
| 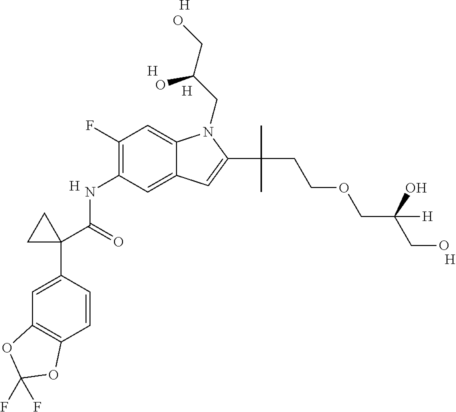 | 308 |
| 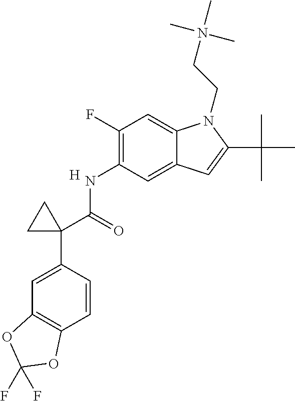 | 309 |
| 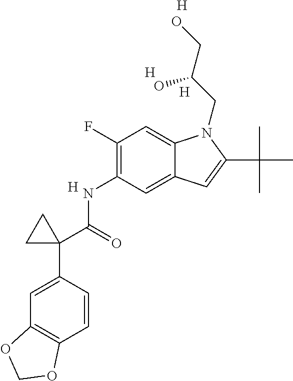 | 310 |
| 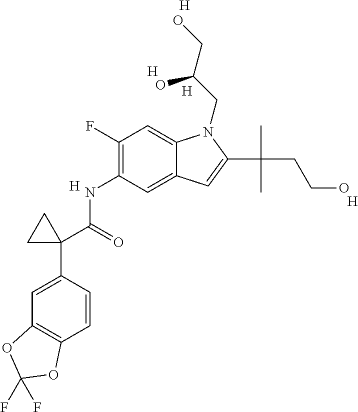 | 311 |
| 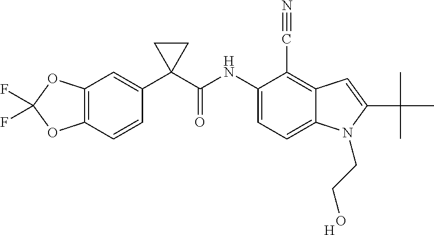 | 312 |
| 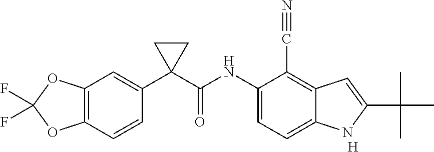 | 313 |
| 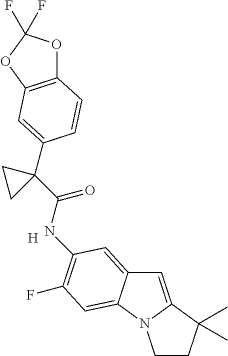 | 314 |
| 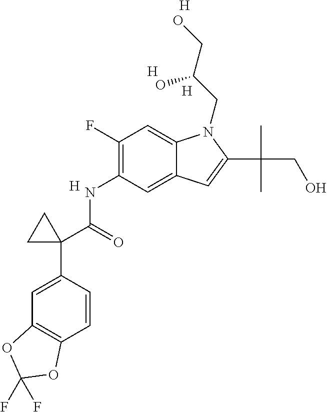 | 315 |
| 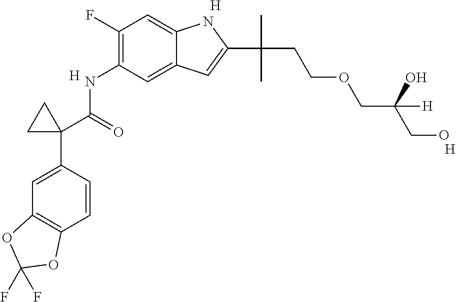 | 316 |
| 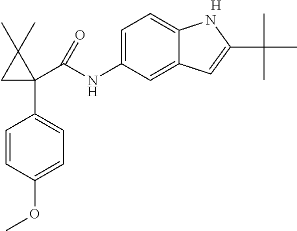 | 317 |
| 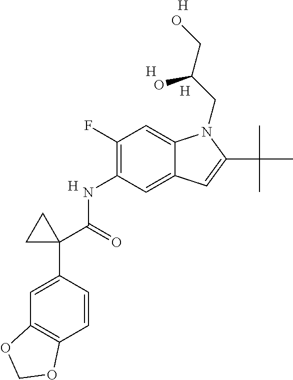 | 318 |
| 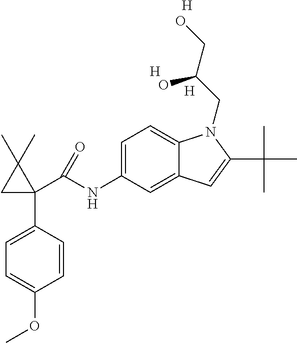 | 319 |
| 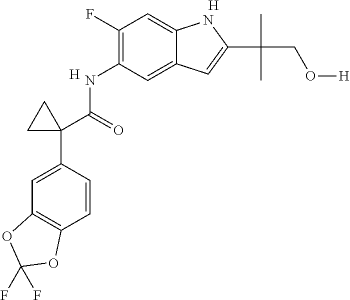 | 320 |
| 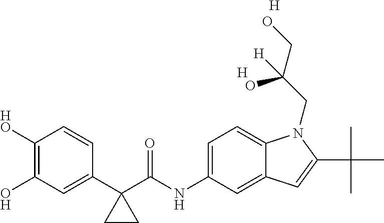 | 321 |
| 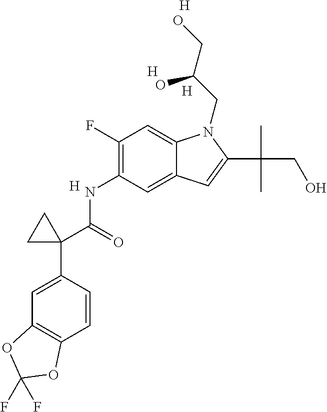 | 322 |

Another aspect of the present invention provides a compound that is useful for modulating ABC transporter activity. The compound has Formula DIc:

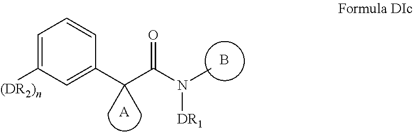

or a pharmaceutically acceptable salt thereof.

DR₁, DR₂, and ring A are defined above in Formula D, and ring B, DR₃ and p are defined in Formula DIa. Furthermore, when ring A is unsubstituted cyclopentyl, n is 1, DR₂ is 4-chloro, and DR₁ is hydrogen, then ring B is not 2-(tertbutyl)indol-5-yl, or (2,6-dichlorophenyl(carbonyl))-3-methyl-1H-indol-5-yl; and when ring A is unsubstituted cyclopentyl, n is 0, and DR₁ is hydrogen, then ring B is not

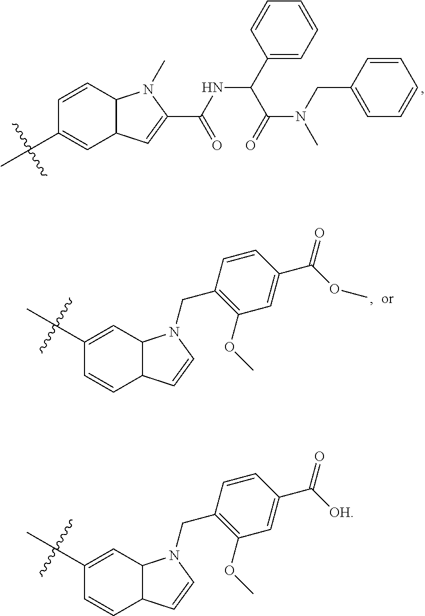 [SMILES: *C1=CC2C=C(C(=O)NC(C(=O)N(C)Cc3ccccc3)c3ccccc3)N(C)C2C=C1 ; *C1=CC2C(C=C1)C=CN2Cc1ccc(C(=O)OC)cc1OC ; *C1=CC2C(C=C1)C=CN2Cc1ccc(C(=O)O)cc1OC]

Another aspect of the present invention provides a compound that is useful for modulating ABC transporter activity. The compound has Formula DId:

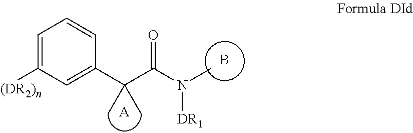

or a pharmaceutically acceptable salt thereof.

DR₁, DR₂, and ring A are defined above in Formula D, and ring B, DR₃ and p are defined in Formula DIa.

However, when DR₁ is H, n is 0, ring A is an unsubstituted cyclopentyl, and ring B is an indole-5-yl substituted with 1-2 of DR₃, then each DR₃ is independently —Z^{G}DR₁₂, where each Z^{G} is independently a bond or an unsubstituted branched or straight C₁₋₆ aliphatic chain wherein up to two carbon units of Z^{G} are optionally and independently replaced by —CS—, —CONDR^{G}NDR^{G}—, —CO₂—, —OCO—, —NDR^{G}CO₂—, —O—, —NDR^{G}CONDR^{G}—, —OCONDR^{G}—, —NDR^{G}NDR^{G}—, —S—, —SO—, —SO₂—, —NDR^{G}—, —SO₂NDR^{G}—, —NDR^{G}SO₂—, or —NDR^{G}SO₂NDR^{C}—, each DR₁₂ is independently DR^{G}, halo, —OH, —NH₂, —NO₂, —CN, or —OCF₃, and each DR^{G} is independently hydrogen, an unsubstituted aliphatic, an optionally substituted cycloaliphatic, an optionally substituted heterocycloaliphatic, an unsubstituted aryl, or an optionally substituted heteroaryl; or any two adjacent DR₃ groups together with the atoms to which they are attached form an optionally substituted heterocycle. Furthermore, when DR₁ is H, n is 1, DR₂ is 4-chloro, ring A is an unsubstituted cyclopentyl, and ring B is an indole-5-yl substituted with 1-2 of DR₃, then each DR₃ is independently —Z^{H}DR₂₂, where each Z^{H} is independently a bond or an unsubstituted branched or straight C₁₋₃ aliphatic chain wherein up to two carbon units of Z^{H} are optionally and independently replaced by —CS—, —CONDR^{H}NDR^{H}, —CO₂—, —OCO—, —NDR^{H}CO₂—, —O—, —NDR^{H}CONDR^{H}—, —OCONDR^{H}—, —NDR^{H}NDR^{H}—, —S—, —SO—, —SO₂—, —NDR^{H}—, —SO₂NDR^{H}—, —NDR^{H}SO₂—, or —NDR^{H}SO₂NDR^{H}—, each DR₂₂ is independently DR^{H}, halo, —OH, —NH₂, —NO₂, —CN, or —OCF₃, and each DR^{H} is independently hydrogen, a substituted C₄ alkyl, an optionally substituted C2-6 alkenyl, an optionally substituted C2-6 alkynyl, an optionally substituted C₄ alkenyl, an optionally substituted C₄ alkynyl, an optionally substituted cycloaliphatic, an optionally substituted heterocycloaliphatic, an optionally substituted heteroaryl, an unsubstituted phenyl, or a mono-substituted phenyl, or any two adjacent DR₃ groups together with the atoms to which they are attached form an optionally substituted heterocycle.

Another aspect of the present invention provides a compound that is useful for modulating ABC transporter activity. The compound has Formula DII:

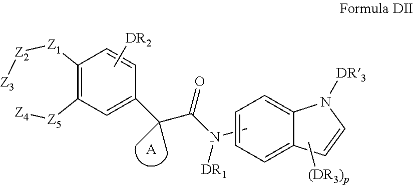

or a pharmaceutically acceptable salt thereof.

DR₁, DR₂, and ring A are defined above in formula DI; DR₃, DR′₃, and p are defined above in Formula DIa; and Z₁, Z₂, Z₃, Z₄, and Z₅ are defined above in Formula DIb.

Another aspect of the present invention provides a compound that is useful for modulating ABC transporter activity. The compound has Formula DIIa:

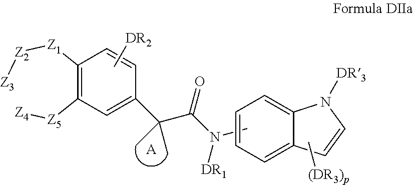

or a pharmaceutically acceptable salt thereof.

DR₁, DR₂, and ring A are defined above in Formula D; DR₃, DR′₃, and p are defined above in Formula DIa; and Z₁, Z₂, Z₃, Z₄, and Z₅ are defined above in Formula DIb.

Another aspect of the present invention provides a compound that is useful for modulating ABC transporter activity. The compound has Formula DIIb:

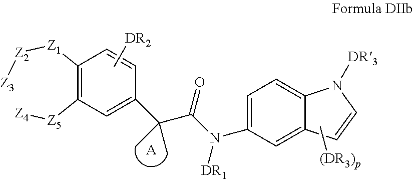

or a pharmaceutically acceptable salt thereof.

DR₁, DR₂, and ring A, are defined above in Formula D; DR₃, DR′₃, and p are defined above in Formula DIa; and Z₁, Z₂, Z₃, Z₄, and Z₅ are defined above in Formula DIb.

Another aspect of the present invention provides a compound that is useful for modulating ABC transporter activity. The compound has Formula DIIc:

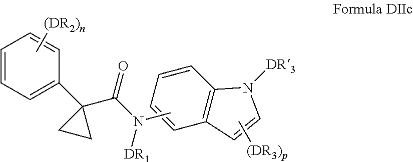

or a pharmaceutically acceptable salt thereof.

DR₁, DR₂ and n are defined above in Formula D; and DR₃, DR′₃, and p are defined in formula DIa.

Another aspect of the present invention provides a compound that is useful for modulating ABC transporter activity. The compound has Formula DIId:

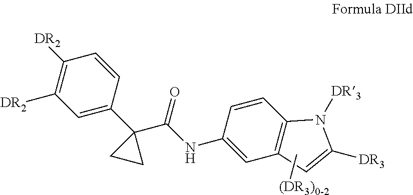

or a pharmaceutically acceptable salt thereof.

Both DR₂ groups, together with the atoms to which they are attached form a group selected from:

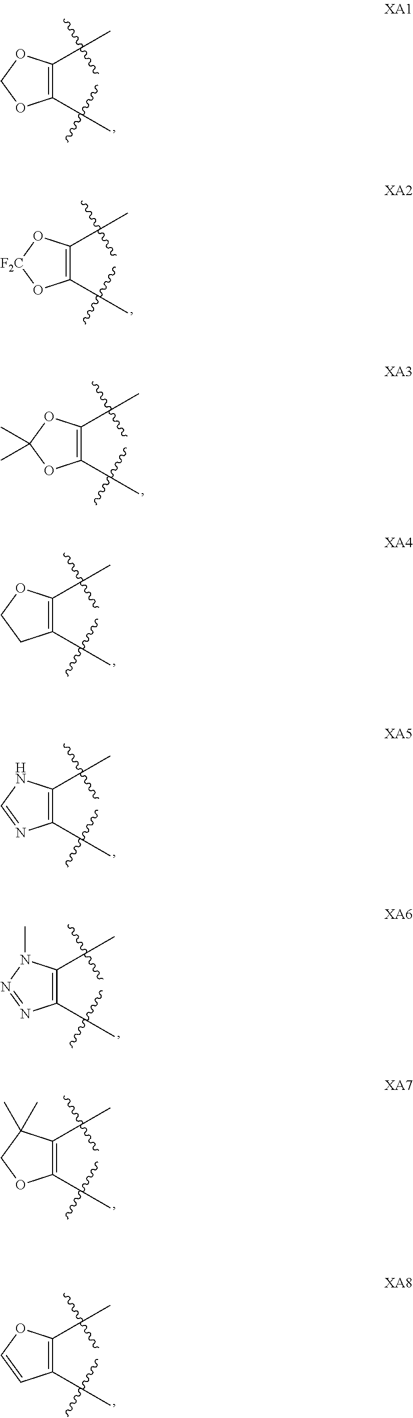

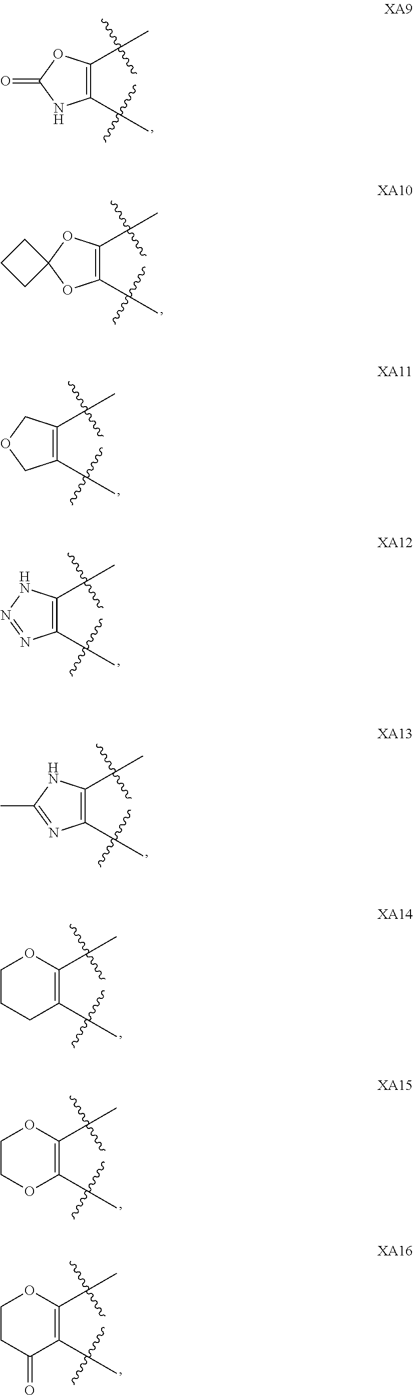

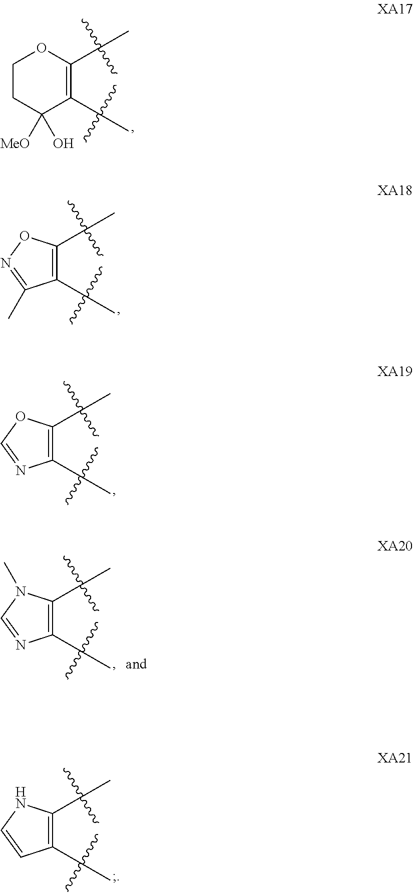

DR₃ is independently selected from one of the following:

### —H, —CH₃, —CH₂CH₃, —C(O)CH₃, —CH₂CH₂OH, —C(O)OCH₃,

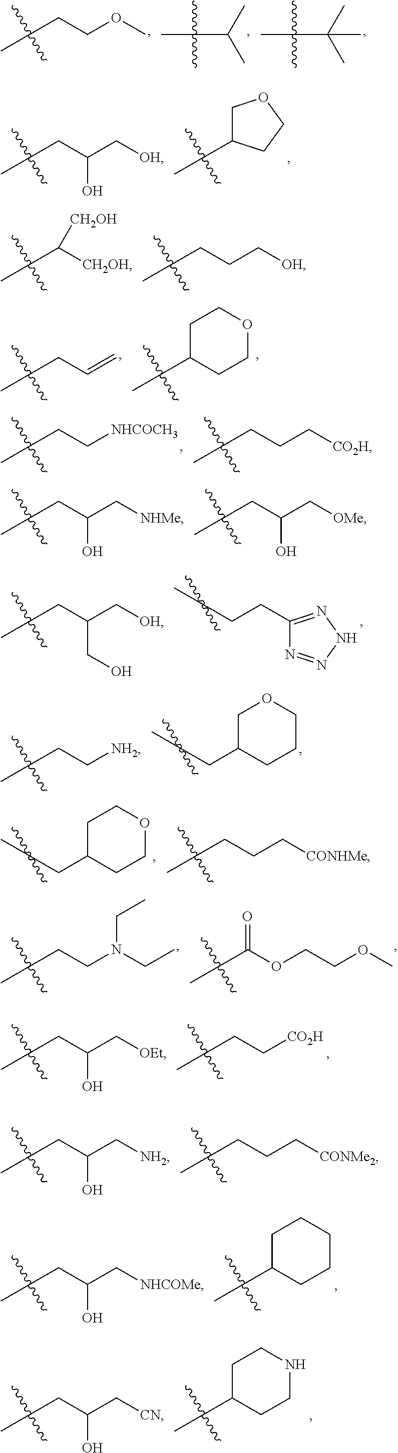

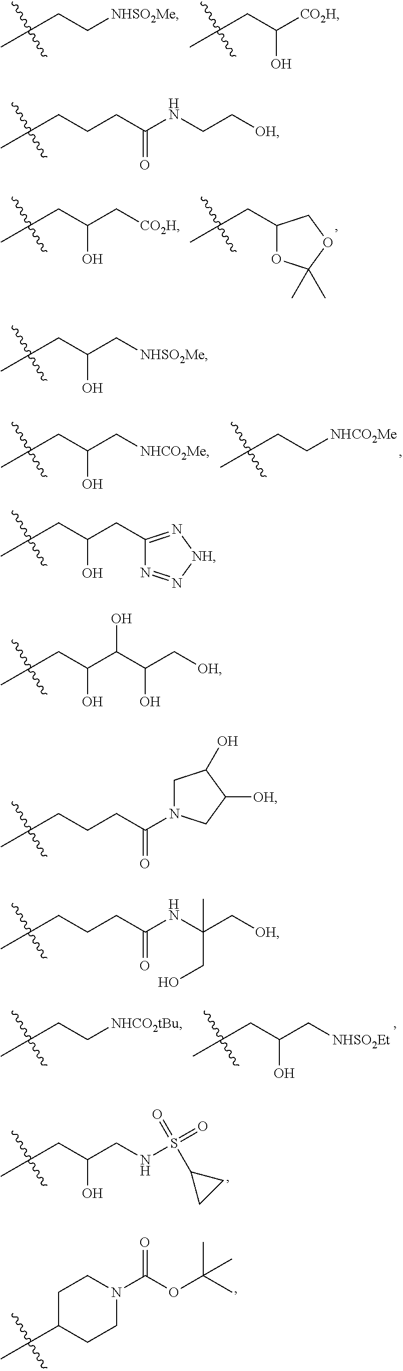

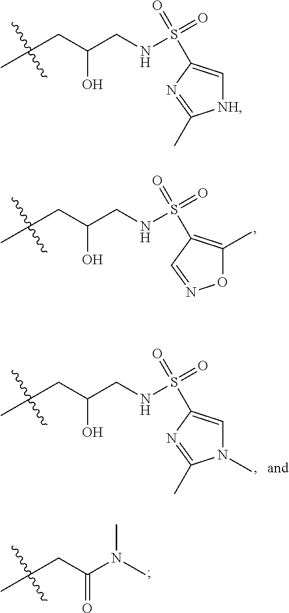

and each DR₃ is independently selected from —H, —CH₃, —CH₂OH, —CH₂CH₃, —CH₂CH₂OH, —CH₂CH₂CH₃, —NH₂, halo, —OCH₃, —CN, —CF₃, —C(O)OCH₂CH₃, —S(O)2CH₃, —CH₂NH₂, —C(O)NH₂,

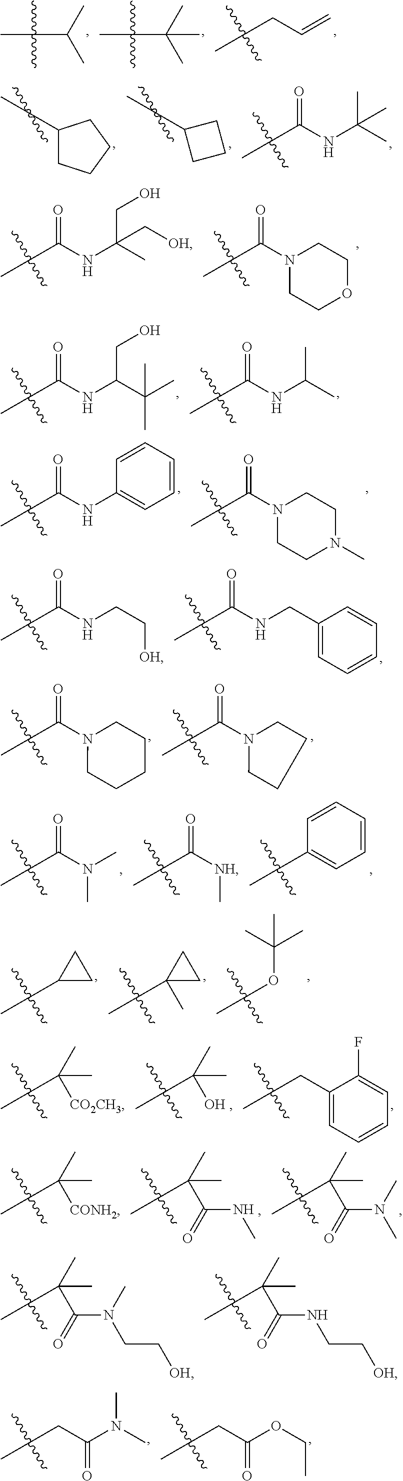

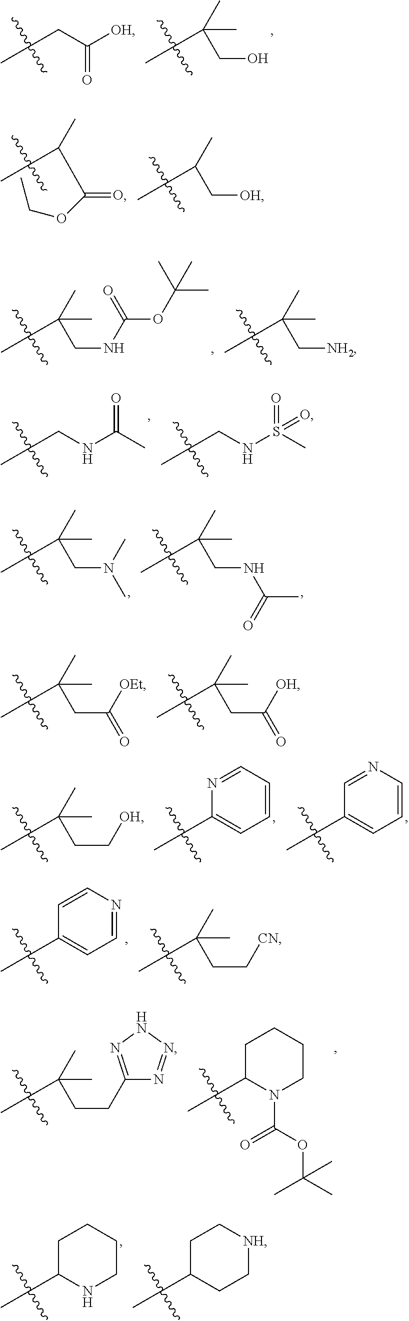

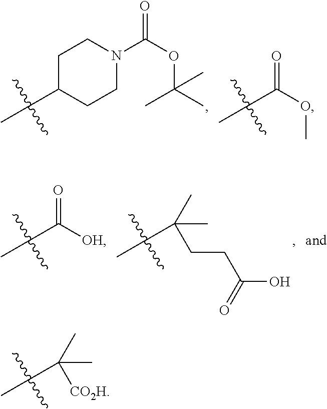

### IV. Generic Synthetic Schemes

The Column D compounds of Formulae (D, DIc, DId, DII, DIIa, DIIb, DIIc, and DIId) may be readily synthesized from commercially available or known starting materials by known methods. Exemplary synthetic routes to produce compounds of Formulae (D, DIc, DId, DII, DIIa, DIIb, DIIc, and DIId) are provided below in Schemes 1-22 below.

Preparation of the compounds of the invention is achieved by the coupling of a ring B amine with a ring A carboxylic acid as illustrated in Scheme 1.

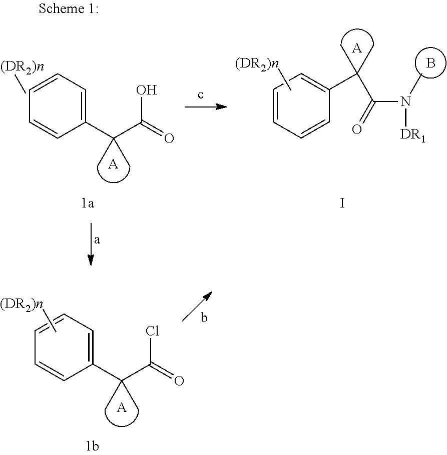

Referring to Scheme 1, the acid 1a may be converted to the corresponding acid chloride 1b using thionyl chloride in the presence of a catalystic amount of dimethylformamide. Reaction of the acid chloride with the amine

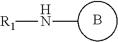

provides compounds of the invention I. Alternatively, the acid 1a may be directly coupled to the amine using known coupling reagents such as, for example, HATU in the presence of triethylamine.

Preparation of the acids 1a may be achieved as illustrated in Scheme 2.

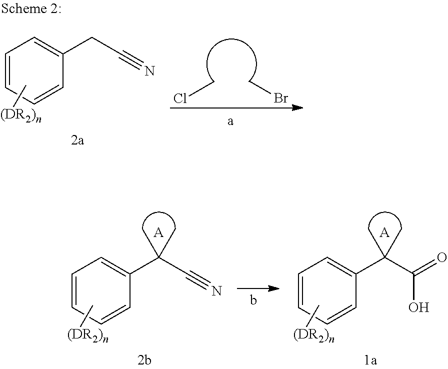

a) NaOH, BTEAC; b) NaOH, Δ

Referring to Scheme 2, the nitrile 2a reacts with a suitable bromochloroalkane in the presence of sodium hydroxide and a phase transfer catalyst such as butyltriethylammonium chloride to provide the intermediate 2b. Hydrolysis of the nitrile of 2b provides the acid 1a. In some instances, isolation of the intermediate 2b is unnecessary.

The phenylacetonitriles 2a are commercially available or may be prepared as illustrated in Scheme 3.

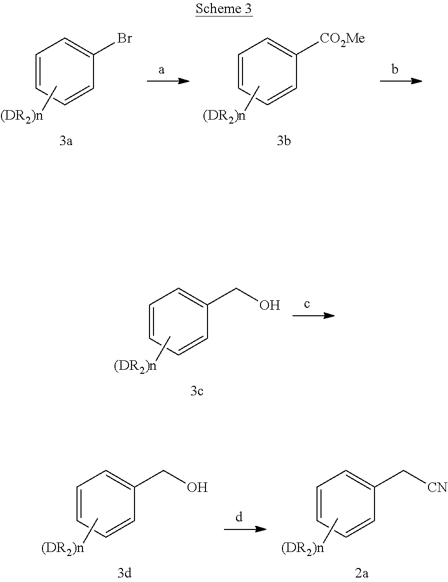

a) Pd(PPh₃)₄, CO, MeOH; b) LiAlH₄, THF; c) SOCl₂; d) NaCN

Referring to Scheme 3, reaction of an aryl bromide 3a with carbon monoxide in the presence of methanol and tetrakis(triphenylphosphine)palladium (0) provides the ester 3b. Reduction of 3b with lithium aluminum hydride provides the alcohol 3c which is converted to the halide 3d with thionyl chloride. Reaction of 3d with sodium cyanide provides the nitrile 2a.

Other methods of producing the nitrile 2a are illustrated in schemes 4 and 5 below.

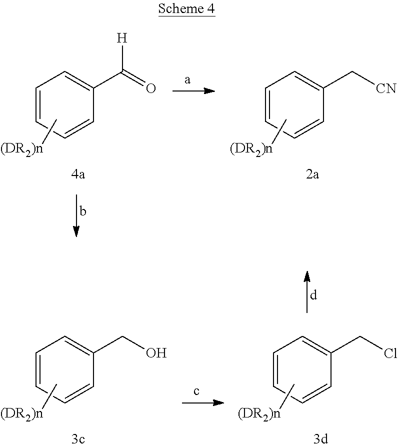

a) TosMIC; b) NaBH₄, THF; c) SOCl₂; d) NaCN

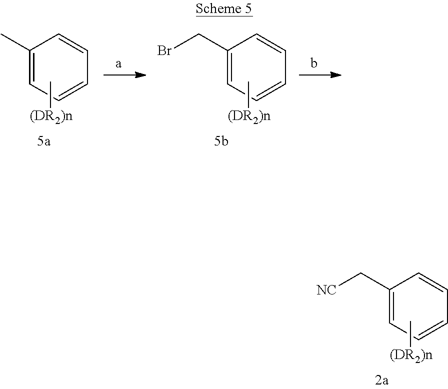

a) NBS, AIBN, CCl₄; b) NaCN, EtOH

Preparation of

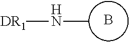

components is illustrated in the schemes that follow. A number of methods for preparing ring B compounds wherein ring B is an indole have been reported. See for example Angew. Chem. 2005, 44, 606; J. Am. Chem. Soc. 2005, 127, 5342,); J. Comb. Chem. 2005, 7, 130; Tetrahedron 2006, 62, 3439; J. Chem. Soc. Perkin Trans. 1, 2000, 1045.

One method for preparing

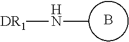

is illustrated in Scheme 6.

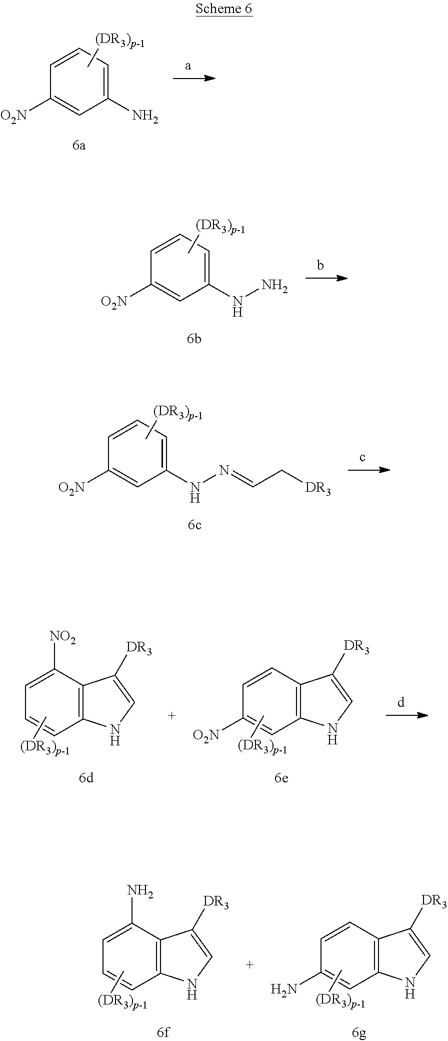

a) NaNO₂, HCl, SnCl₂; b) NaOH, DR₃CH₂C(O)DR₃, EtOH; c) H₃PO₄, toluene; d) H₂, Pd—C, EtOH

Referring to Scheme 6, a nitroaniline 6a is converted to the hydrazine 6b using nitrous acid in the presence of HCl and stannous chloride. Reaction of 6b with an aldehyde or ketone CH₃C(O)DR₃ provides the hydrazone 6c which on treatment with phosphoric acid in toluene leads to a mixture of nitro indoles 6d and 6e. Catalytic hydrogenation in the presence of palladium on carbon provides a mixture of the amino indoles 6f and 6 g which may be separated using know methods such as, for example, chromatography.

An alternative method is illustrated in scheme 7.

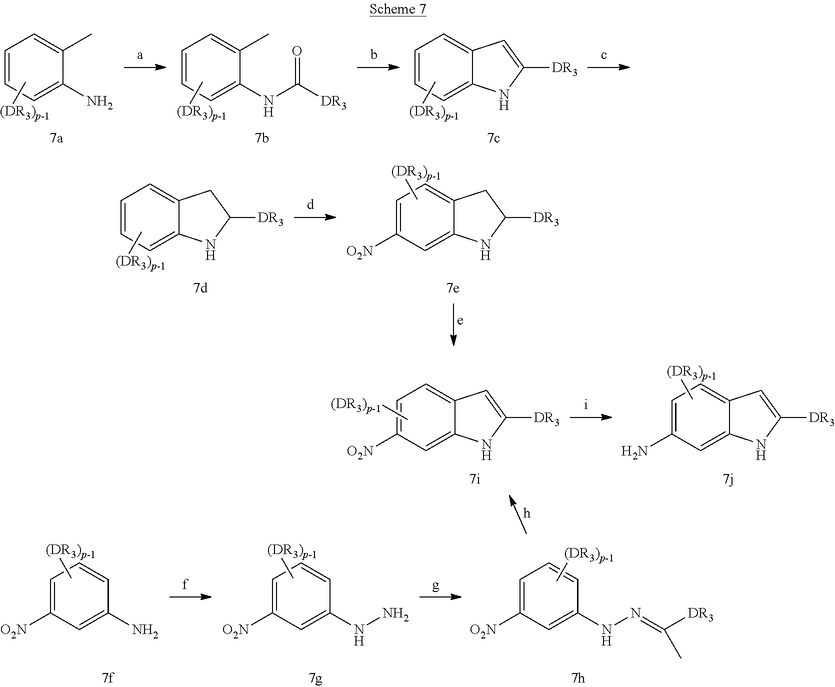

a) DR₃ₐCOCl, Et₃N, CH₂Cl₂; b) n-BuLi, THF; c) NaBH₄, AcOH; d) KNO₃, H₂SO₄; e) DDQ, 1,4-dioxane; f) NaNO₂, HCl, SnCl₂.2H₂O, H₂O; g) MeCODR₃, EtOH; h) PPA; i) Pd/C, EtOH or H₂, Raney Ni, EtOH or MeOH

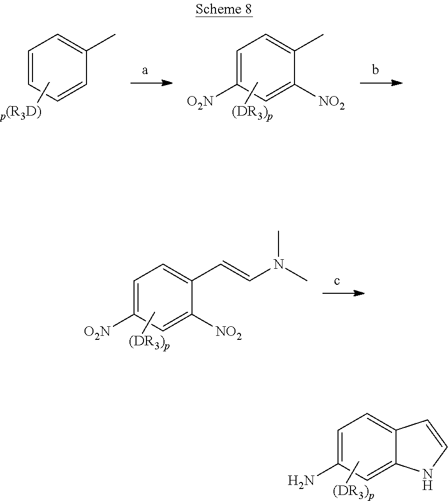

a) HNO₃, H₂SO₄; b) Me₂NCH(OMe)₂, DMF; c) H₂, Raney Ni, EtOH

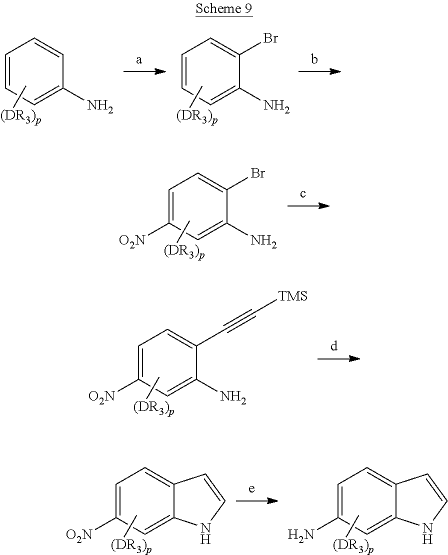

a) NBS, DMF; b) KNO₃, H₂SO₄; c) HC═C-TMS, Pd(PPh₃)2Cl₂, CuI, Et₃N, toluene, H₂O; d) CuI, DMF; e) H₂, Raney Ni, MeOH

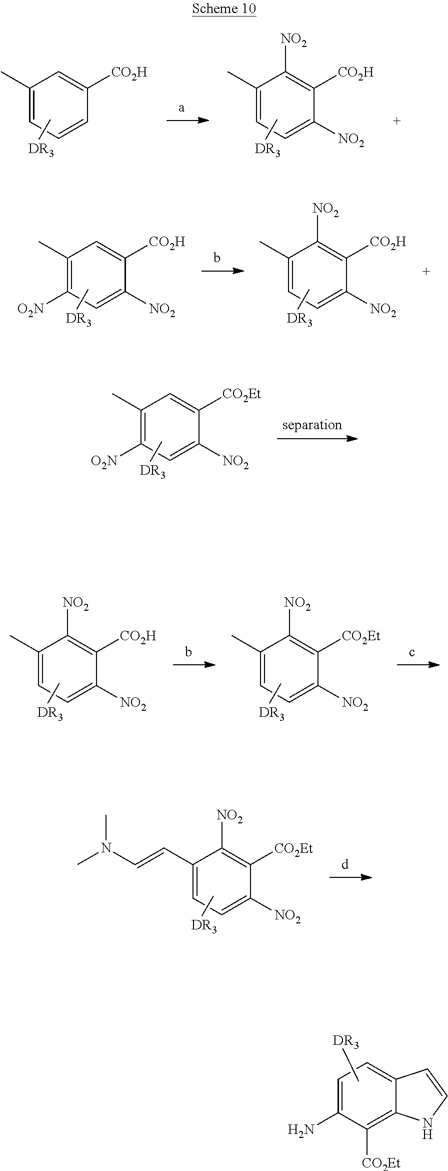

a) HNO₃, H₂SO₄; b) SOCl₂; EtOH; c) DMA, DMF; d) Raney Ni, H₂, MeOH

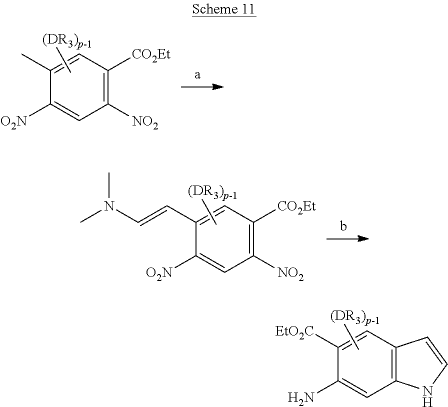

a) DMA, DMF; b) Raney Ni, H₂, MeOH

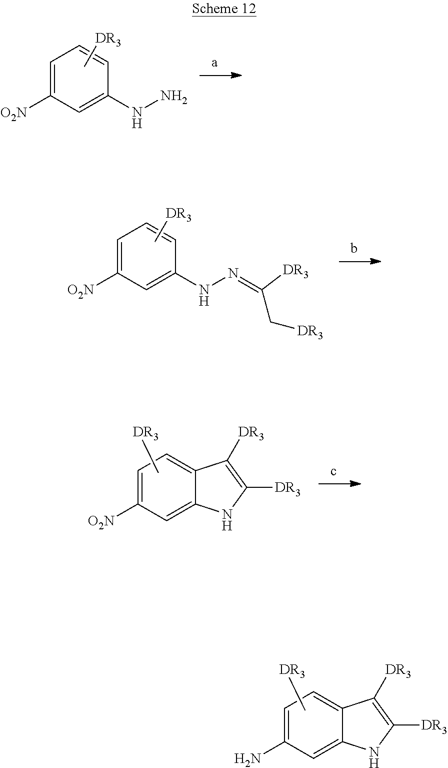

a) DR₃ₐCH₂COR_{3b}, AcOH, EtOH; b) H₃PO₄, toluene; c) H₂, Pd/C, EtOH

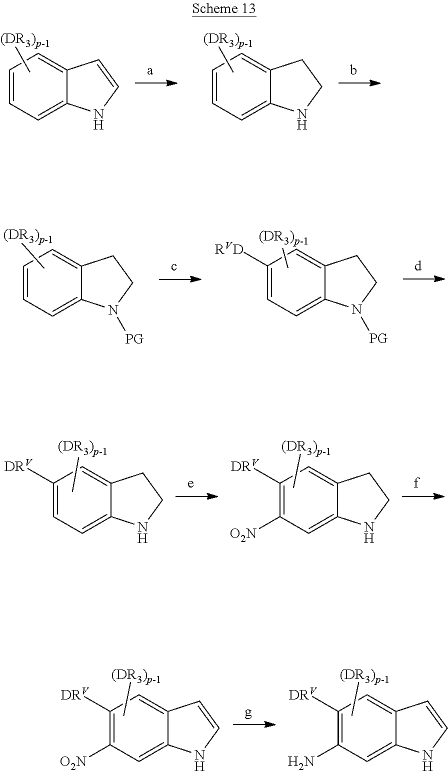

a) NaBH₃CN; b) When PG=SO₂Ph: PhSO₂Cl, Et₃N, DMAP, CH₂Cl₂; When PG=Ac: AcCl, NaHCO₃, CH₂Cl₂; c) When DR^{V}=RCO: (RCO)2O, AlCl₃, CH₂Cl₂; When DR^{V}=Br: Br₂, AcOH; d) HBr or HCl; e) KNO₃, H₂SO₄; 1) MnO₂, CH₂Cl₂ or DDQ, 1,4-dioxane; g) H₂, Raney Ni, EtOH.

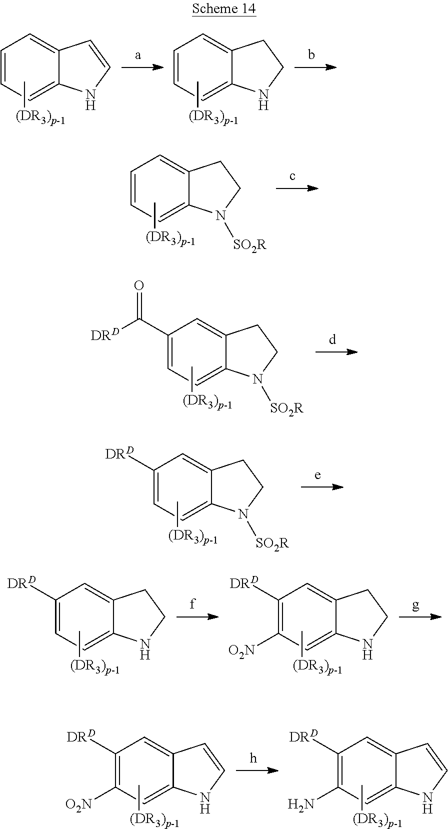

a) NaBH₃CN; b) RSO₂Cl, DMAP, Et₃N, CH₂Cl₂; c) DR^{D}C(O)Cl, AlCl₃, CH₂Cl₂; d) NaBH₄, THF; e) HBr; f) KNO₃, H₂SO₂; g) MnO₂; g) Raney Ni, H₂, EtOH

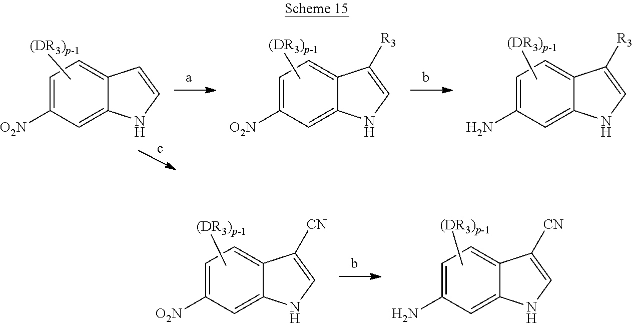

a) R₃X (X=Br, I), zinc triflate, TBAI, DIEA, toluene; b) H₂, Raney Ni, EtOH or H₂, Pd/C, EtOH or SnCl₂.2H₂O, EtOH; c) ClSO₂NCO, DMF, CH₃CN

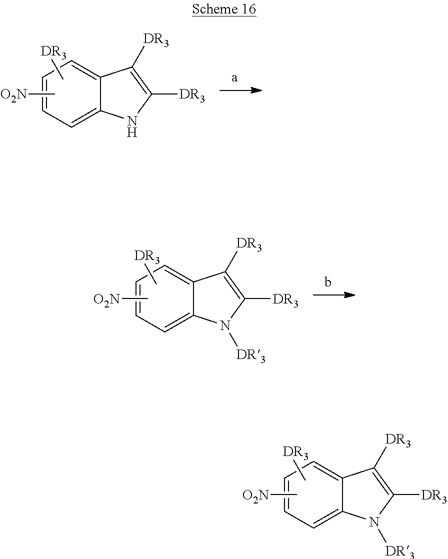

a) when X=Cl, Br, I, or OTs: DR′₃X, K₂CO₃, DMF or CH₃CN; b) H₂, Pd/C, EtOH or SnCl₂.2H₂O, EtOH or SnCl₂.2H₂O, DIEA, EtOH.

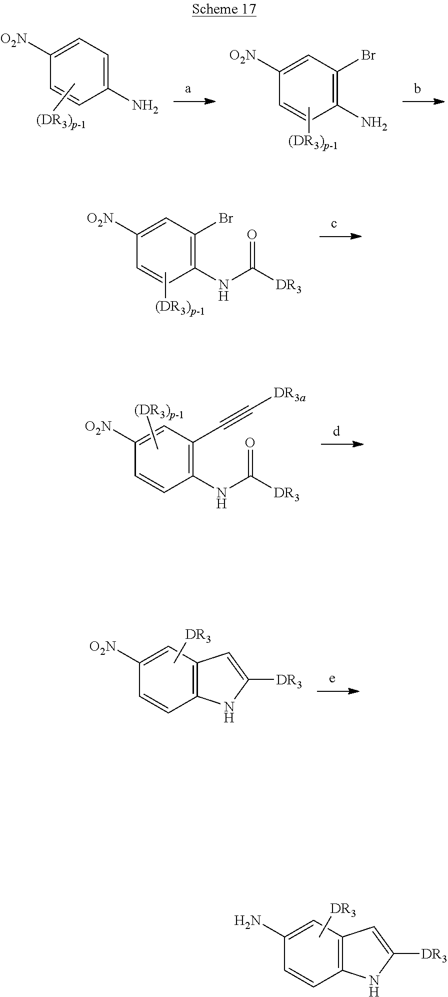

a) Br₂, AcOH; b) RC(O)Cl, Et₃N, CH₂Cl₂; c) HC═CDR₃ₐ, Pd(PPh₃)₂Cl₂, CuI, Et₃N; d) TBAF, THF or tBuOK, DMF or Pd(PPh₃)₂Cl₂, CuI, DMF; e) H₂, Pd/C, EtOH or SnCl₂, MeOH or HCO₂NH₄, Pd/C, EtOH

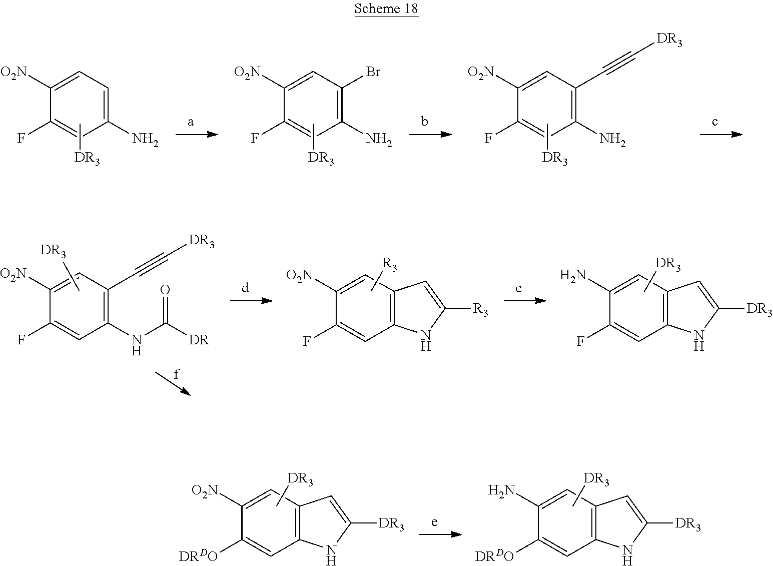

a) Br₂, AcOH, CHCl₃; b) DR₃ₐC═CH, CuI, Et₃N, Pd(PPh₃)₂Cl₂; c) DRCOCl, Et₃N, CH₂Cl₂; d) TBAF, DMF; e) Raney Ni, H₂, MeOH; I) DROK, DMF

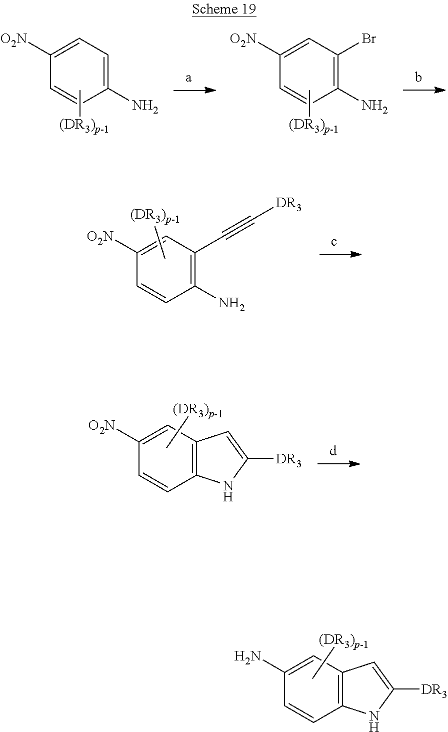

Br₂, AcOH; b) HC═CDR₃ₐ, Pd(PPh₃)₂Cl₂, CuI, Et₃N; c) Pd(PPh₃)2Cl₂, CuI, DMF; d) H₂, Pd/C, EtOH or SnCl₂, MeOH or HCO₂NH₄, Pd/C, EtOH

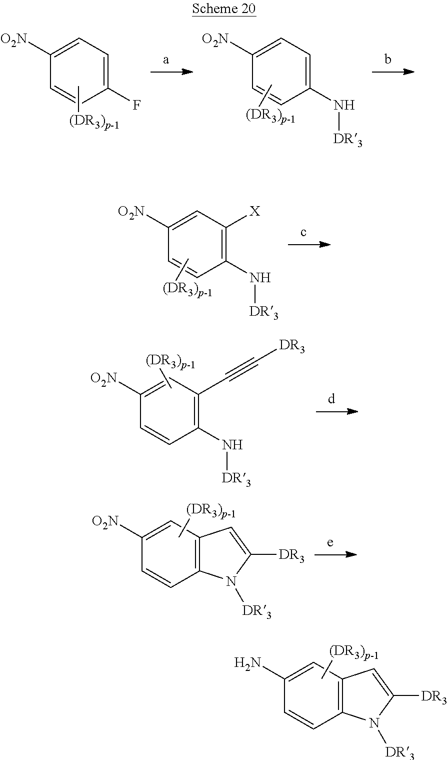

a) H₂NDR′₃; b) X=Br: Br₂, HOAc; X=I: NIS; c) HC═CR₃, Pd(PPh₃)₂Cl₂, CuI, Et₃N; d) CuI, DMF or TBAF, THF; e) H₂, Pd/C, EtOH or SnCl₂, MeOH or HCO₂NH₄, Pd/C, EtOH

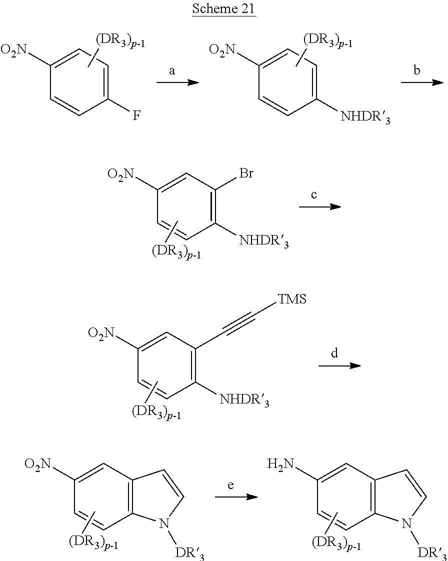

a) DR′₃NH₂, DMSO; b) Br₂, AcOH; c) TMS-C═CH, CuI, TEA, Pd(PPh₃)₂Cl₂; d) CuI, DMSO; e) Raney Ni, H₂, MeOH

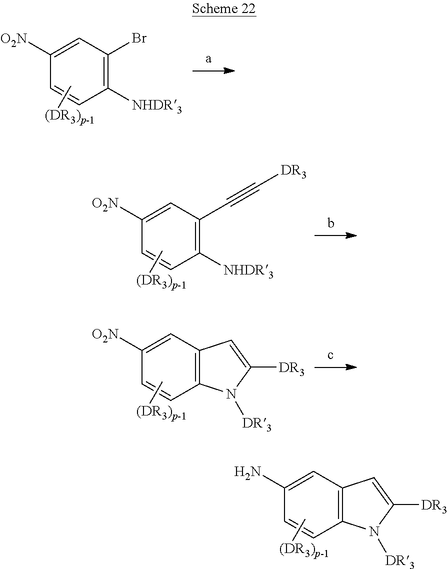

a) DR₃ₐC═CH, CuI, TEA, Pd(PPh₃)₂Cl₂; b) TBAF, THF; c) Raney Ni, MeOH

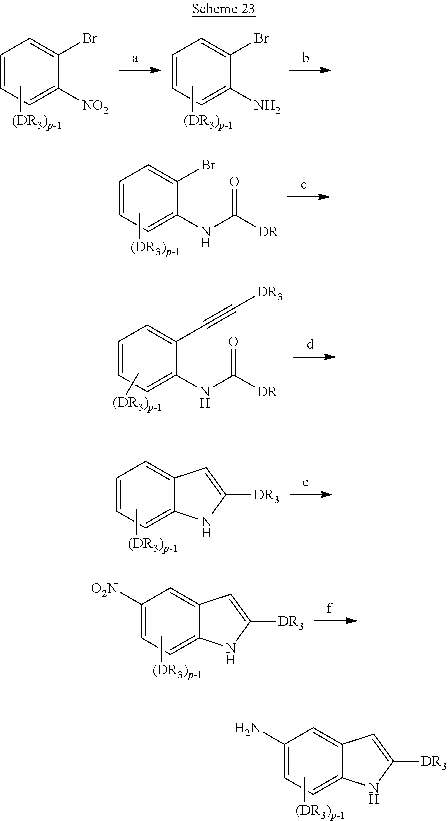

a) NaBH₄, NiCl₂, MeOH; b) DRC(O)Cl; c) Pd(PPh₃)Cl₂, HC═C-DR₃, CuI, Et₃N; d) tBuOK, DMF; e) KNO₃, H₂SO₄; f) NaBH₄, NiCl₂, MeOH

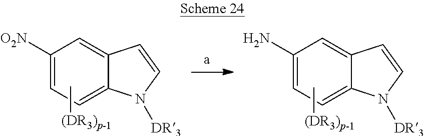

a) SnCl₂, EtOH or Pd/C, HCO₂NH₄ or H₂, Pd/C, EtOH or Raney Ni, H₂, EtOH

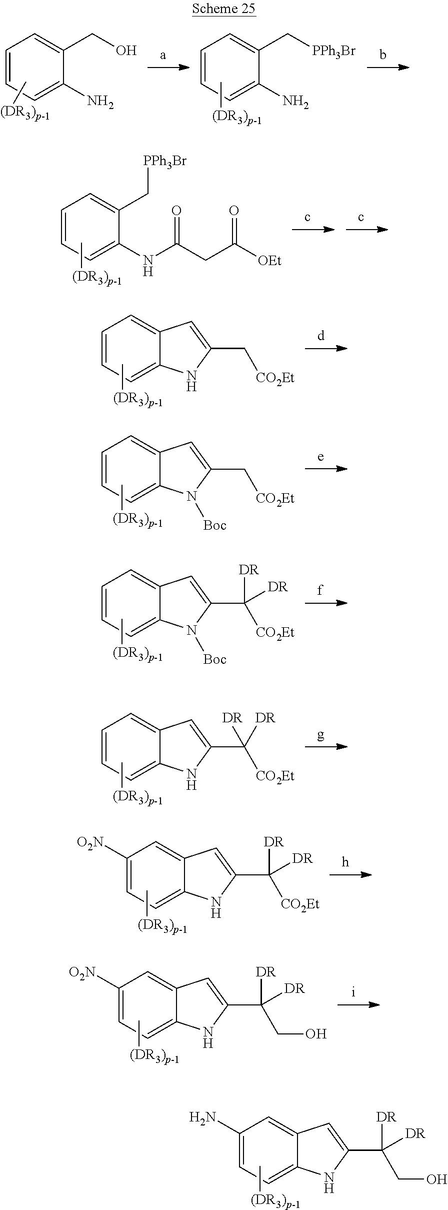

a) PPh₃, HBr; b) Cl(O)CCH₂CO₂Et; c) tBuOK; d) (Boc)₂O, DMAP; e) KHMDS, DR-X; KHMDS, DR-X; f) TFA; g) NaNO₃, H₂SO₄; h) LiAlH₄, THF; i) SnCl₂, EtOH

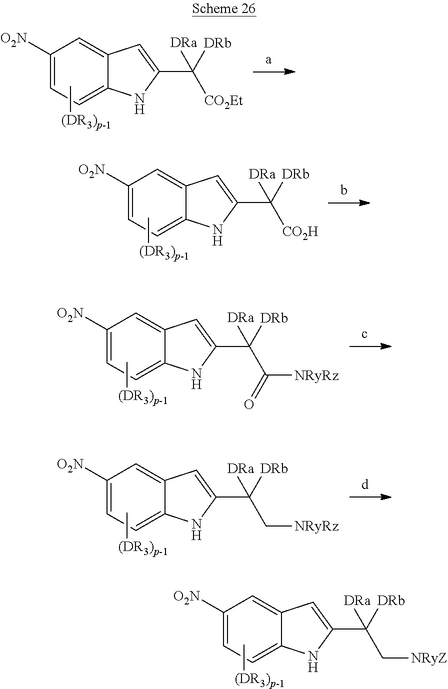

a) LiOH; b) EDC, HOBt, Et₃N, HNDRyDRz; c) BH₃-THF; d) if DRz=H, DRC(O)Cl(Z=DRC(O)—) or DRSO₂Cl (Z=DRSO₂—) or DRO(CO)Cl (Z=DRO(CO)—) or (DRO(CO))₂O (Z═Z=DRO(CO)—), Et₃N, CH₂Cl₂

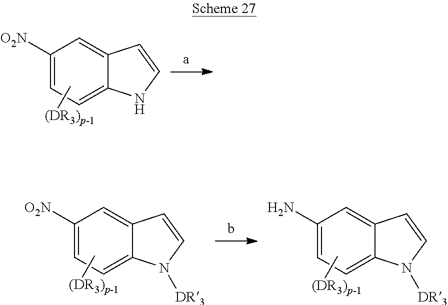

a) DR′₃—X (X=Br, I, or OTs), base (K₂CO₃ or Cs₂CO₃), DMF or CH₃CN; b) H₂, Pd/C, EtOH or Pd/C, HCO₂NH₄

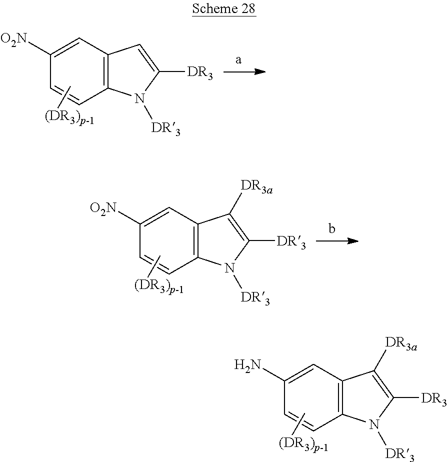

### a) DR₃ₐX (X=Cl, Br, I), AlCl₃, CH₂Cl₂; b) Raney Ni, H₂, MeOH

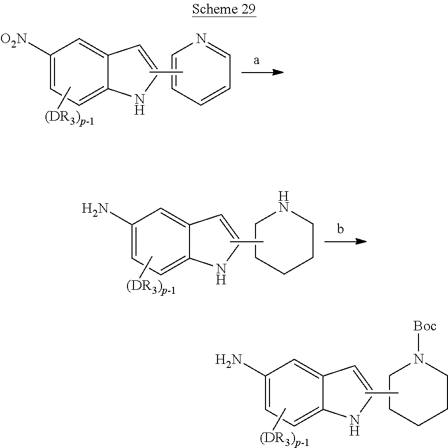

### a) Ha/MeOH; PtO₂, H₂; b) (Boc)₂O, Et₃N, THF

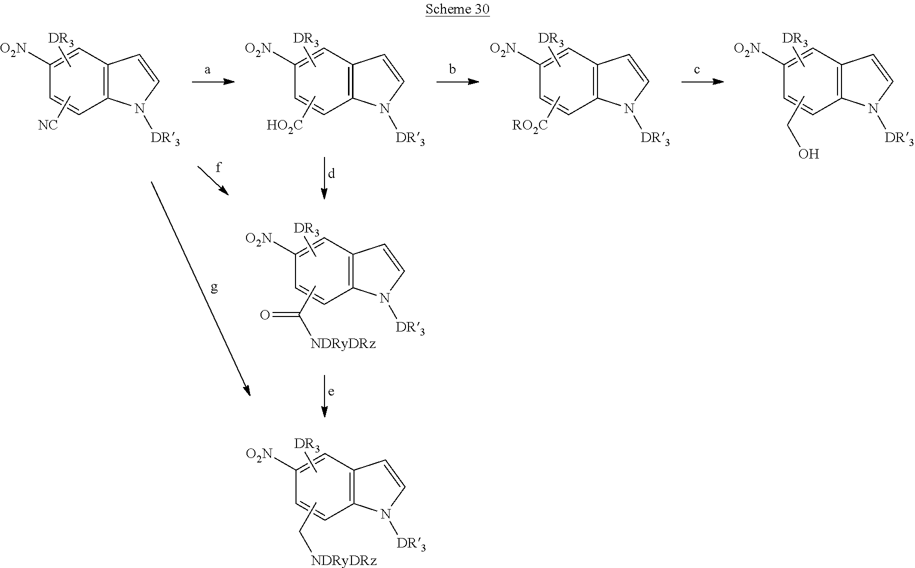

a) NaOH or LiOH; b) DROH, HCl; c) NaBH₄ or LiAlH₄ or DIBAL-H, THF; d) HNDRyDRz, HATU, Et₃N, EtOH or DMF; e) LiAlH₄, THF or BH₃-THF; f) H₂O₂, H₂O (DRy=DRz=H); g) H₂, Pd/C

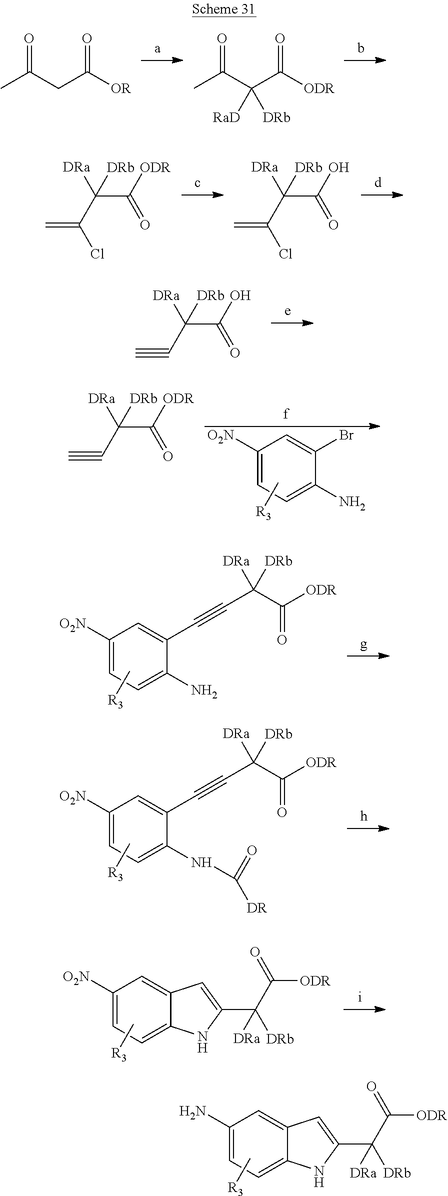

a) DRₐ—X, NaH; DR_{b}—X, NaH; b) PCl₅, CH₂Cl₂; c) NaOH; d) NaNH₂, DMSO; e) CH₂N₂; f) Pd(PPh₃)4, CuI, Et₃N; g) DRC(O)Cl, pyr, CH₂Cl₂; h) Pd(CH₃CN)₂Cl₂, CH₃CN; i) Raney Ni, H₂, MeOH

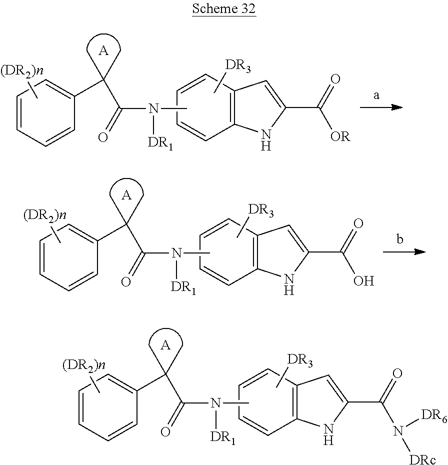

a) LiOH, THF/H₂O; b) HNDRyDRz, HATU, TEA, DMF/CH₂Cl₂

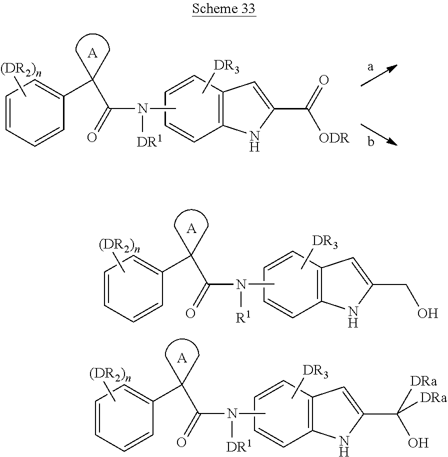

### a) LiBH₄, THF/H₂O or LiAlH₄, THF; b) Rₐ—Li, THF

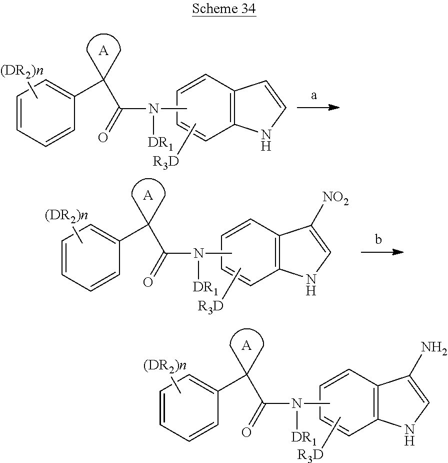

a) NaNO₂, AcOH/H₂O; b) Zn, AcOH

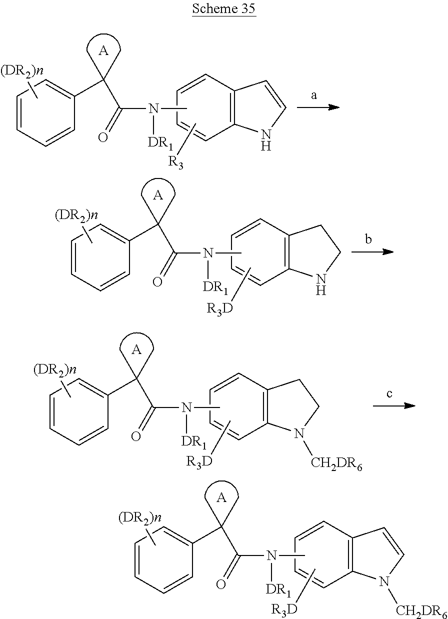

a) NaBH₃CN; b) DR₆CHO, NaHB(OAc)₃, TFA, DCE; c) chloranil or CDCl₃, light or DDQ

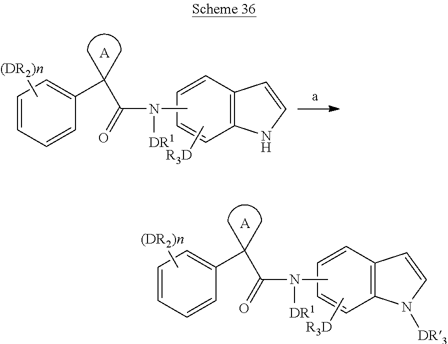

### a) NaH, DMF-THF; DR′₃—X (X=Cl, Br, I, or OTs)

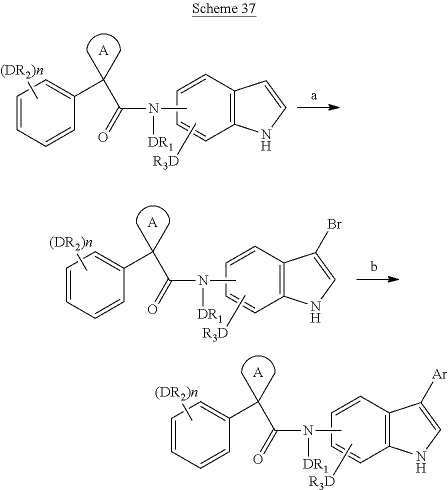

### a) NBS; b) Ar—B(ODR)₂, Pd-FibreCat 1007, K₂CO₃, EtOH

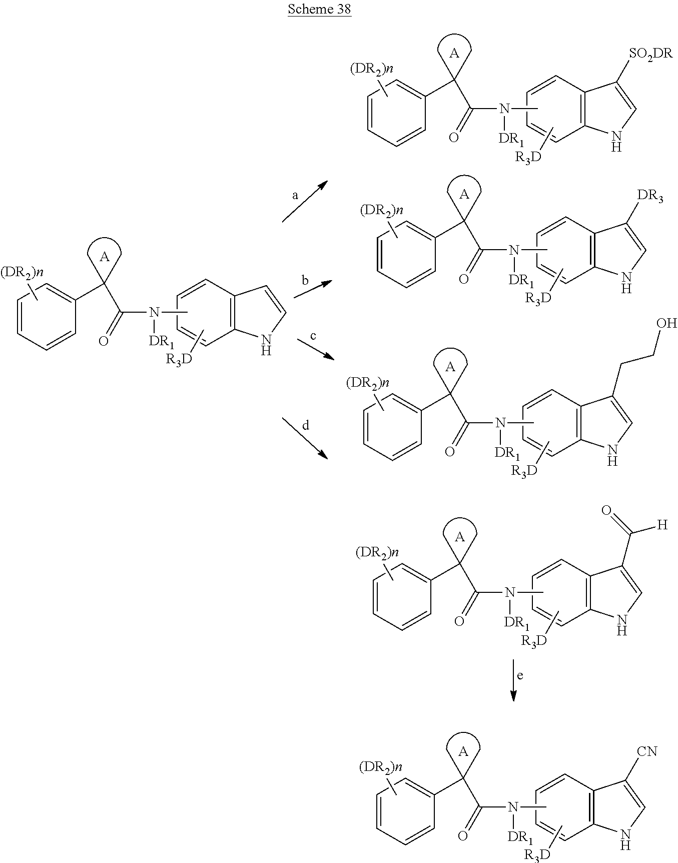 [1574] a) DRSO₂Cl, NaH, THF-DMF; b) DR₃—X (X=Br, I, or OTs), NaH, THF-DMF; c) ethylene dioxide, InCl₃; d) POCl₃, DMF; e) H₂N—OH, CH₂Cl₂; Ac₂O

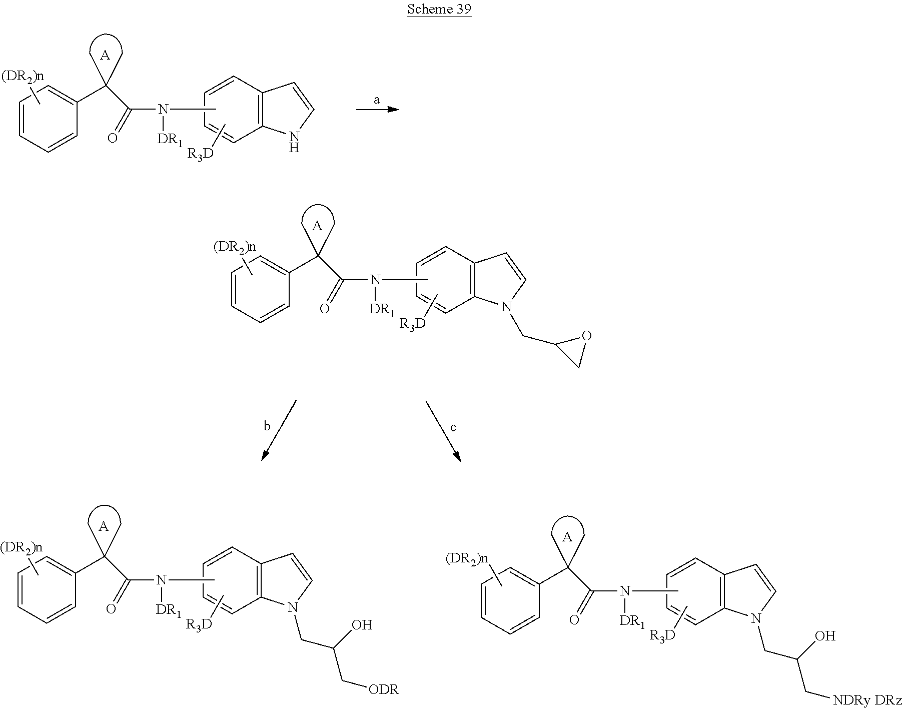

a) NaH, THF-DMF; epichlorohydrin; b) ROH; c) HNDRyDRz

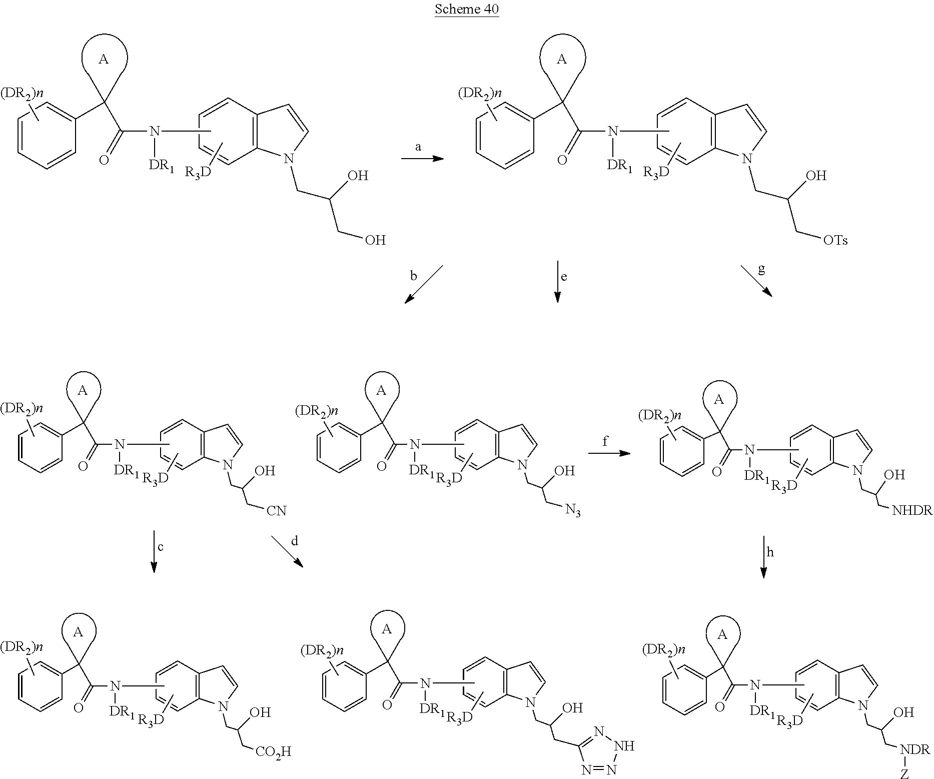

a) TsCl, Et₃N, CH₂Cl₂; b) NaCN, DMF; c) NaOH, MeOH; d) NaN₃, NH₄Cl; e) NaN₃, DMF; f) Pd/C, H₂, MeOH (DR=H); h) DR^{x}C(O)Cl (Z=DR^{x}C(O)—) or DH^{x}SO₂Cl(Z=DR^{x}SO₂—) or DR^{x}O(CO)Cl (Z=DR^{x}O(CO)—) or (DR^{x}O(CO))₂O (Z=DR^{x}O(CO)—), Et₃N, CH₂Cl₂

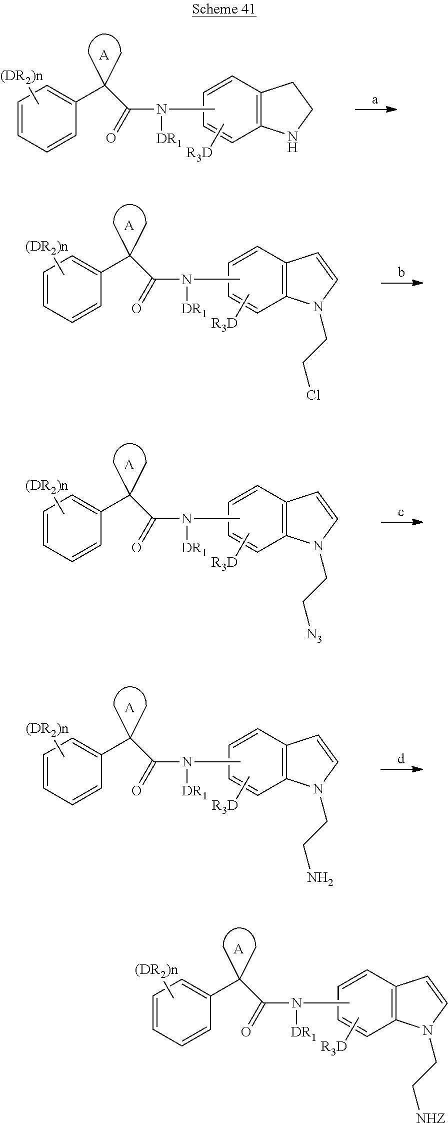

a) ClCH₂CHO, NaHB(OAc)₃, CH₂Cl₂; CDCl₃, light; b) NaN₃, NaI, DMF; c) H₂, Pd/C, MeOH, AcOH; d) DRC(O)Cl (Z=DRC(O)—) or DRSO₂Cl (Z=DRSO₂—) or DRO(CO)Cl (Z=DRO(CO)—) or (DRO(CO))₂O (Z=DRO(CO)—), Et₃N, CH₂Cl₂

In the schemes above, the radical DR employed therein is a substituent, e.g., DRW as defined hereinabove. One of skill in the art will readily appreciate that synthetic routes suitable for various substituents of the present invention are such that the reaction conditions and steps employed do not modify the intended substituents.

### VI. Preparations and Examples

### General Procedure I

### Carboxylic Acid Building Block

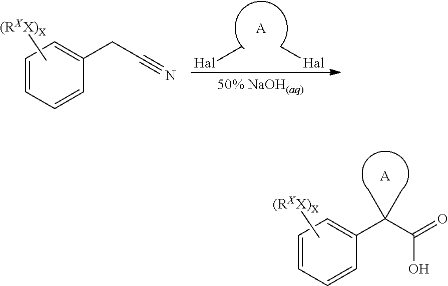 [1577] Hal=Cl, Br, I

Benzyltriethylammonium chloride (0.025 equivalents) and the appropriate dihalo compound (2.5 equivalents) were added to a substituted phenyl acetonitrile. The mixture was heated at 70° C. and then 50% sodium hydroxide (10 equivalents) was slowly added to the mixture. The reaction was stirred at 70° C. for 12-24 hours to ensure complete formation of the cycloalkyl moiety and then heated at 130° C. for 24-48 hours to ensure complete conversion from the nitrile to the carboxylic acid. The dark brown/black reaction mixture was diluted with water and extracted with dichloromethane three times to remove side products. The basic aqueous solution was acidified with concentrated hydrochloric acid to pH less than one and the precipitate which began to form at pH 4 was filtered and washed with 1 M hydrochloric acid two times. The solid material was dissolved in dichloromethane and extracted two times with 1 M hydrochloric acid and one time with a saturated aqueous solution of sodium chloride. The organic solution was dried over sodium sulfate and evaporated to dryness to give the cycloalkylcarboxylic acid. Yields and purities were typically greater than 90%.

### Example 1

### 1-Benzo[1,3]dioxol-5-yl-cyclopropanecarboxylic acid

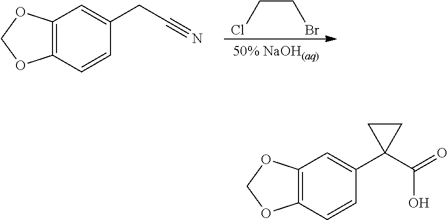

A mixture of 2-(benzo[d][1,3]dioxol-5-yl)acetonitrile (5.10 g 31.7 mmol), 1-bromo-2-chloro-ethane (9.00 mL 109 mmol), and benzyltriethylammonium chloride (0.181 g, 0.795 mmol) was heated at 70° C. and then 50% (wt./wt.) aqueous sodium hydroxide (26 mL) was slowly added to the mixture. The reaction was stirred at 70° C. for 24 hours and then heated at 130° C. for 48 hours. The dark brown reaction mixture was diluted with water (400 mL) and extracted once with an equal volume of ethyl acetate and once with an equal volume of dichloromethane. The basic aqueous solution was acidified with concentrated hydrochloric acid to pH less than one and the precipitate filtered and washed with 1 M hydrochloric acid. The solid material was dissolved in dichloromethane (400 mL) and extracted twice with equal volumes of 1 M hydrochloric acid and once with a saturated aqueous solution of sodium chloride. The organic solution was dried over sodium sulfate and evaporated to dryness to give a white to slightly off-white solid (5.23 g, 80%) ESI-MS m/z calc. 206.1. found 207.1 (M+1)⁺. Retention time 2.37 minutes. ¹H NMR (400 MHz, DMSO-d₆) δ 1.07-1.11 (m, 2H), 1.38-1.42 (m, 2H), 5.98 (s, 2H), 6.79 (m, 2H), 6.88 (m, 1H), 12.26 (s, 1H).

### General Procedure II

### Carboxylic Acid Building Block

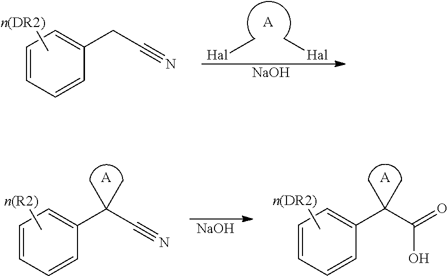 [1582] Hal=Cl, Br, I, all other variables are as defined in the text.

Sodium hydroxide (50% aqueous solution, 7.4 equivalents) was slowly added to a mixture of the appropriate phenyl acetonitrile, benzyltriethylammonium chloride (1.1 equivalents), and the appropriate dihalo compound (2.3 equivalents) at 70° C. The mixture was stirred overnight at 70° C. and the reaction mixture was diluted with water (30 mL) and extracted with ethyl acetate. The combined organic layers were dried over sodium sulfate and evaporated to dryness to give the crude cyclopropanecarbonitrile, which was used directly in the next step.

The crude cyclopropanecarbonitrile was refluxed in 10% aqueous sodium hydroxide (7.4 equivalents) for 2.5 hours. The cooled reaction mixture was washed with ether (100 mL) and the aqueous phase was acidified to pH 2 with 2M hydrochloric acid. The precipitated solid was filtered to give the cyclopropanecarboxylic acid as a white solid.

### General Procedure III

### Carboxylic Acid Building Block

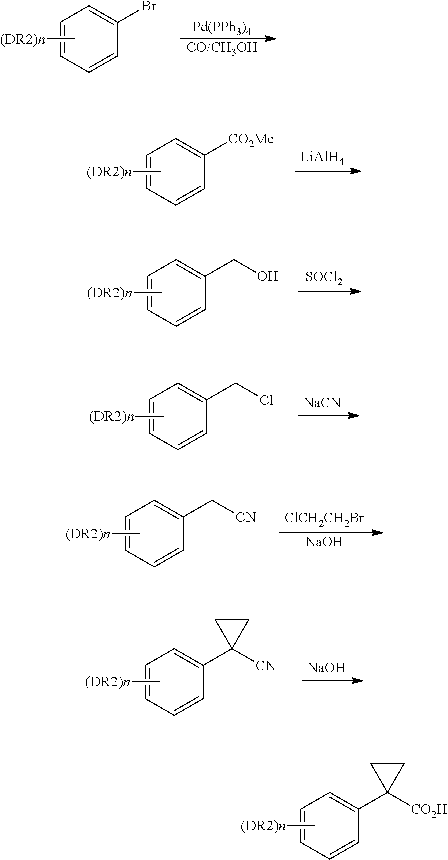

### Example 2

### 1-(2,2-Difluoro-benzo[1,3]dioxol-5-yl)-cyclopropanecarboxylic acid

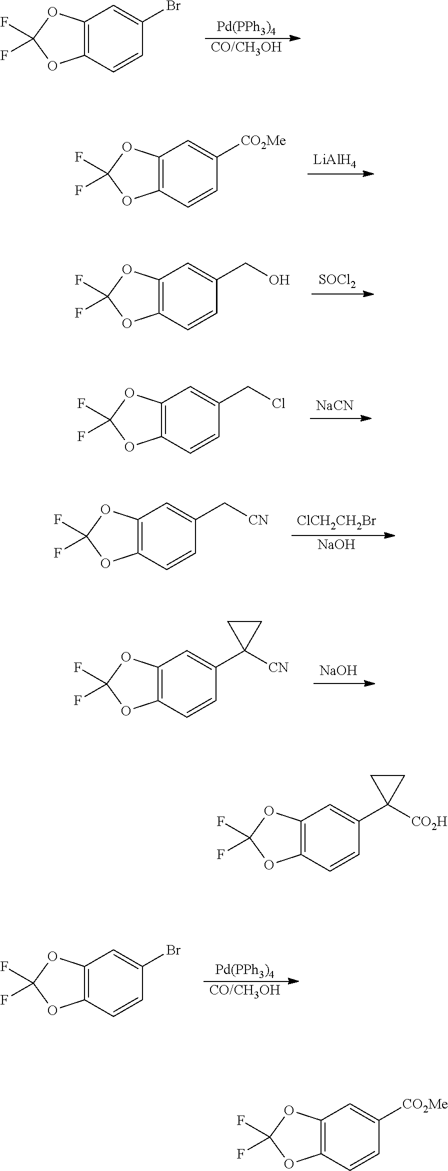

### 2,2-Difluoro-benzo[1,3]dioxole-5-carboxylic acid methyl ester

A solution of 5-bromo-2,2-difluoro-benzo[1,3]dioxole (11.8 g, 50.0 mmol) and tetrakis(triphenylphosphine)palladium (0) [Pd(PPh₃)₄, 5.78 g, 5.00 mmol] in methanol (20 mL) containing acetonitrile (30 mL) and triethylamine (10 mL) was stirred under a carbon monoxide atmosphere (55 PSI) at 75° C. (oil bath temperature) for 15 hours. The cooled reaction mixture was filtered and the filtrate was evaporated to dryness. The residue was purified by silica gel column chromatography to give crude 2,2-difluoro-benzo[1,3]dioxole-5-carboxylic acid methyl ester (11.5 g), which was used directly in the next step.

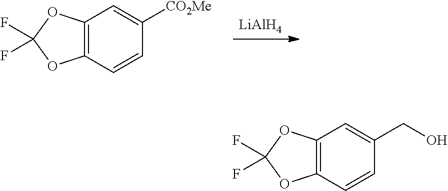 [SMILES: COC(=O)c1ccc2c(c1)OC(F)(F)O2 ; "LiAlH4" ; OCc1ccc2c(c1)OC(F)(F)O2]

### (2,2-Difluoro-benzo[1,3]dioxol-5-yl)-methanol

Crude 2,2-difluoro-benzo[1,3]dioxole-5-carboxylic acid methyl ester (11.5 g) dissolved in 20 mL of anhydrous tetrahydrofuran (THF) was slowly added to a suspension of lithium aluminum hydride (4.10 g, 106 mmol) in anhydrous THF (100 mL) at 0° C. The mixture was then warmed to room temperature. After being stirred at room temperature for 1 hour, the reaction mixture was cooled to 0° C. and treated with water (4.1 g), followed by sodium hydroxide (10% aqueous solution, 4.1 mL). The resulting slurry was filtered and washed with THF. The combined filtrate was evaporated to dryness and the residue was purified by silica gel column chromatography to give (2,2-difluoro-benzo[1,3]dioxol-5-yl)-methanol (7.2 g, 38 mmol, 76% over two steps) as a colorless oil.

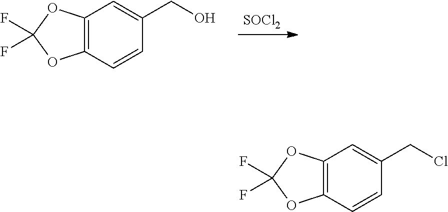

### 5-Chloromethyl-2,2-difluoro-benzo[1,3]dioxole

Thionyl chloride (45 g, 38 mmol) was slowly added to a solution of (2,2-difluoro-benzo[1,3]dioxol-5-yl)-methanol (7.2 g, 38 mmol) in dichloromethane (200 mL) at 0° C. The resulting mixture was stirred overnight at room temperature and then evaporated to dryness. The residue was partitioned between an aqueous solution of saturated sodium bicarbonate (100 mL) and dichloromethane (100 mL). The separated aqueous layer was extracted with dichloromethane (150 mL) and the organic layer was dried over sodium sulfate, filtrated, and evaporated to dryness to give crude 5-chloromethyl-2,2-difluoro-benzo[1,3]dioxole (4.4 g) which was used directly in the next step.

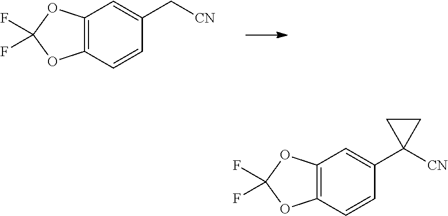 [SMILES: N#CCc1ccc2c(c1)OC(F)(F)O2 ; N#CC1(c2ccc3c(c2)OC(F)(F)O3)CC1]

### (2,2-Difluoro-benzo[1,3]dioxol-5-yl)-acetonitrile

A mixture of crude 5-chloromethyl-2,2-difluoro-benzo[1,3]dioxole (4.4 g) and sodium cyanide (1.36 g, 27.8 mmol) in dimethylsulfoxide (50 mL) was stirred at room temperature overnight. The reaction mixture was poured into ice and extracted with ethyl acetate (300 mL). The organic layer was dried over sodium sulfate and evaporated to dryness to give crude (2,2-difluoro-benzo[1,3]dioxol-5-yl)-acetonitrile (3.3 g) which was used directly in the next step.

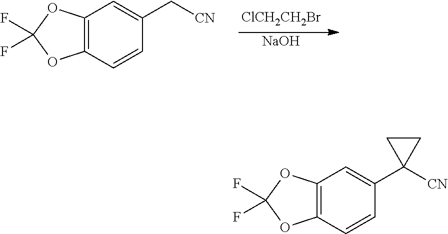

### 1-(2,2-Difluoro-benzo[1,3]dioxol-5-yl)-cyclopropanecarbonitrile

Sodium hydroxide (50% aqueous solution, 10 mL) was slowly added to a mixture of crude (2,2-difluoro-benzo[1,3]dioxol-5-yl)-acetonitrile, benzyltriethylammonium chloride (3.00 g, 15.3 mmol), and 1-bromo-2-chloroethane (4.9 g, 38 mmol) at 70° C.

The mixture was stirred overnight at 70° C. before the reaction mixture was diluted with water (30 mL) and extracted with ethyl acetate. The combined organic layers were dried over sodium sulfate and evaporated to dryness to give crude 1-(2,2-difluoro-benzo[1,3]dioxol-5-yl)-cyclopropanecarbonitrile, which was used directly in the next step.

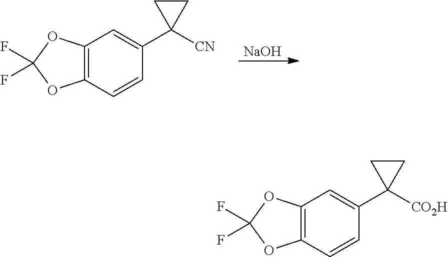

### 1-(2,2-Difluoro-benzo[1,3]dioxol-5-yl)-cyclopropanecarboxylic acid

1-(2,2-Difluoro-benzo[1,3]dioxol-5-yl)-cyclopropanecarbonitrile (crude from the last step) was refluxed in 10% aqueous sodium hydroxide (50 mL) for 2.5 hours. The cooled reaction mixture was washed with ether (100 mL) and the aqueous phase was acidified to pH 2 with 2M hydrochloric acid. The precipitated solid was filtered to give 1-(2,2-difluoro-benzo[1,3]dioxol-5-yl)-cyclopropanecarboxylic acid as a white solid (0.15 g, 1.6% over four steps). ESI-MS m/z calc. 242.04. found 241.58 (M+1)⁺; ¹H NMR (CDCl₃) δ 7.14-7.04 (m, 2H), 6.98-6.96 (m, 1H), 1.74-1.64 (m, 2H), 1.26-1.08 (m, 2H).

### Example 3

### 2-(2,2-Dimethylbenzo[d][1,3]dioxol-5-yl)acetonitrile

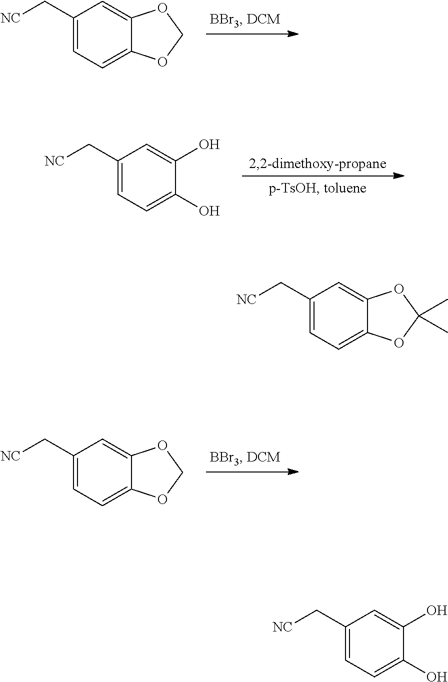

### (3,4-Dihydroxy-phenyl)-acetonitrile

To a solution of benzo[1,3]dioxol-5-yl-acetonitrile (0.50 g, 3.1 mmol) in CH₂Cl₂ (15 mL) was added dropwise BBr₃ (0.78 g, 3.1 mmol) at −78° C. under N₂. The mixture was slowly warmed to room temperature and stirred overnight. H₂O (10 mL) was added to quench the reaction and the CH₂Cl₂ layer was separated. The aqueous phase was extracted with CH₂Cl₂ (2×7 mL). The combined organics were washed with brine, dried over Na₂SO₄ and purified by column chromatography on silica gel (petroleum ether/ethyl acetate 5:1) to give (3,4-dihydroxy-phenyl)-acetonitrile (0.25 g, 54%) as a white solid. ¹H NMR (DMSO-d₆, 400 MHz) δ 9.07 (s, 1H), 8.95 (s, 1H), 6.68-6.70 (m, 2H), 6.55 (dd, J=8.0, 2.0 Hz, 1H), 3.32 (s, 2H).

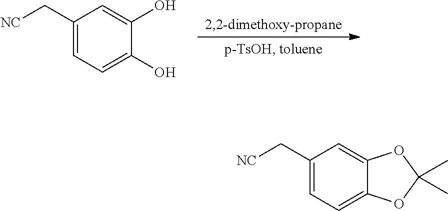

### 2-(2,2-Dimethylbenzo[d][1,3]dioxol-5-yl)acetonitrile

To a solution of (3,4-dihydroxy-phenyl)-acetonitrile (0.20 g, 1.3 mmol) in toluene (4 mL) was added 2,2-dimethoxy-propane (0.28 g, 2.6 mmol) and TsOH (0.010 g, 0.065 mmol). The mixture was heated at reflux overnight. The reaction mixture was evaporated to remove the solvent and the residue was dissolved in ethyl acetate. The organic layer was washed with NaHCO₃ solution, H₂O, brine, and dried over Na₂SO₄. The solvent was evaporated under reduced pressure to give a residue, which was purified by column chromatography on silica gel (petroleum ether/ethyl acetate 10:1) to give 2-(2,2-dimethylbenzo[d][1,3]dioxol-5-yl)acetonitrile (40 mg, 20%). ¹H NMR (CDCl₃, 400 MHz) δ 6.68-6.71 (m, 3H), 3.64 (s, 2H), 1.67 (s, 6H).

### Example 4

### 1-(3,4-Dihydroxy-phenyl)-cyclopropanecarboxylic acid

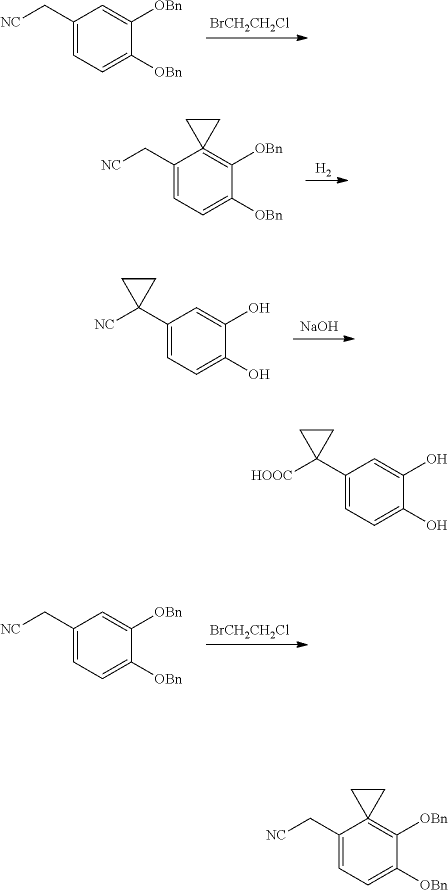

### 1-(3,4-Bis-benzyloxy-phenyl)-cyclopropanecarbonitrile

To a mixture of (n-C₄H₉)₄NBr (0.50 g, 1.5 mmol), toluene (7 mL) and (3,4-bis-benzyloxy-phenyl)-acetonitrile (14 g, 42 mmol) in NaOH (50 g) and H₂O (50 mL) was added BrCH₂CH₂Cl (30 g, 0.21 mol). The reaction mixture was stirred at 50° C. for 5 h before being cooled to room temperature. Toluene (30 mL) was added and the organic layer was separated and washed with H₂O, brine, dried over anhydrous MgSO₄, and concentrated. The residue was purified by column on silica gel (petroleum ether/ethyl acetate 10:1) to give 1-(3,4-bis-benzyloxy-phenyl)-cyclopropanecarbonitrile (10 g, 66%). ¹H NMR (DMSO 300 MHz) δ 7.46-7.30 (m, 10H), 7.03 (d, J=8.4 Hz, 1H), 6.94 (d, J=2.4 Hz, 1H), 6.89 (dd, J=2.4, 8.4 Hz, 1H), 5.12 (d, J=7.5 Hz, 4H), 1.66-1.62 (m, 2H), 1.42-1.37 (m, 2H).

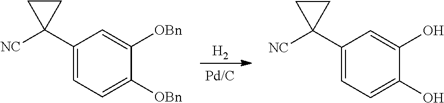 [SMILES: N#CC1(c2ccc(OCc3ccccc3)c(OCc3ccccc3)c2)CC1 ; "H2" ; "Pd/C" ; N#CC1(c2ccc(O)c(O)c2)CC1]

### 1-(3,4-Dihydroxy-phenyl)-cyclopropanecarbonitrile

To a solution of 1-(3,4-bis-benzyloxy-phenyl)-cyclopropanecarbonitrile (10 g, 28 mmol) in MeOH (50 mL) was added Pd/C (0.5 g) under nitrogen atmosphere. The mixture was stirred under hydrogen atmosphere (1 atm) at room temperature for 4 h. The catalyst was filtered off through a celite pad and the filtrate was evaporated under vacuum to give 1-(3,4-dihydroxy-phenyl)-cyclopropanecarbonitrile (4.5 g, 92%). ¹H NMR (DMSO 400 MHz) δ 9.06 (br s, 2H), 6.67-6.71 (m, 2H), 6.54 (dd, J=2.4, 8.4 Hz, 1H), 1.60-1.57 (m, 2H), 1.30-1.27 (m, 2H).

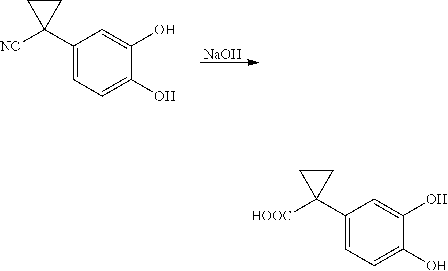 [SMILES: N#CC1(c2ccc(O)c(O)c2)CC1 ; "NaOH" ; O=C(O)C1(c2ccc(O)c(O)c2)CC1]

### 1-(3,4-Dihydroxy-phenyl)-cyclopropanecarboxylic acid

To a solution of NaOH (20 g, 0.50 mol) in H₂O (20 mL) was added 1-(3,4-dihydroxy-phenyl)-cyclopropanecarbonitrile (4.4 g, 25 mmol). The mixture was heated at reflux for 3 h before being cooled to room temperature. The mixture was neutralized with HCl (0.5N) to pH 3-4 and extracted with ethyl acetate (20 mL×3). The combined organic layers were washed with water, brine, dried over anhydrous MgSO₄, and concentrated under vacuum to obtain 1-(3,4-dihydroxy-phenyl)-cyclopropanecarboxylic acid (4.5 g crude). From 900 mg crude, 500 mg pure 1-(3,4-dihydroxy-phenyl)-cyclopropanecarboxylic acid was obtained by preparatory HPLC. ¹H NMR (DMSO, 300 MHz) δ 12.09 (br s, 1H), 8.75 (br s, 2H), 6.50-6.67 (m, 3H), 1.35-1.31 (m, 2H), 1.01-0.97 (m, 2H).

### Example 5

### 1-(2-Oxo-2,3-dihydrobenzo[d]oxazol-5-yl)cyclopropane-carboxylic acid

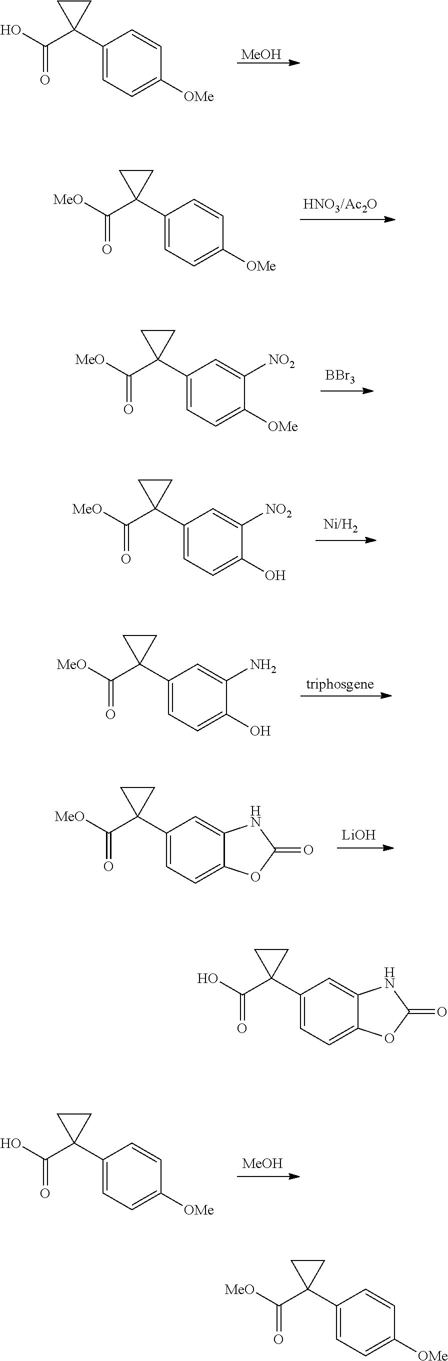

### 1-(4-Methoxy-phenyl)-cyclopropanecarboxylic acid methyl ester

To a solution of 1-(4-methoxy-phenyl)-cyclopropanecarboxylic acid (50 g, 0.26 mol) in MeOH (500 mL) was added toluene-4-sulfonic acid monohydrate (2.5 g, 13 mmol) at room temperature. The reaction mixture was heated at reflux for 20 hours. MeOH was removed by evaporation under vacuum and EtOAc (200 mL) was added. The organic layer was washed with sat. aq. NaHCO₃ (100 mL) and brine, dried over anhydrous Na₂SO₄ and evaporated under vacuum to give 1-(4-methoxy-phenyl)-cyclopropanecarboxylic acid methyl ester (53 g, 99%). ¹H NMR (CDCl₃, 400 MHz) δ 7.25-7.27 (m, 2H), 6.85 (d, J=8.8 Hz, 2H), 3.80 (s, 3H), 3.62 (s, 3 μl), 1.58 (q, J=3.6 Hz, 2H), 1.15 (q, J=3.6 Hz, 2H).

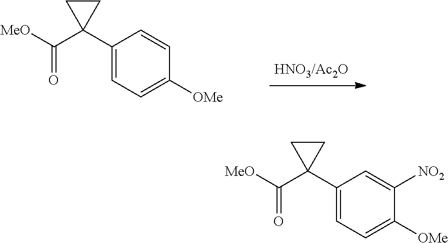 [SMILES: COC(=O)C1(c2ccc(OC)cc2)CC1 ; COC(=O)C1(c2ccc(OC)c([N+](=O)[O-])c2)CC1]

### 1-(4-Methoxy-3-nitro-phenyl)-cyclopropanecarboxylic acid methyl ester

To a solution of 1-(4-methoxy-phenyl)-cyclopropanecarboxylic acid methyl ester (30.0 g, 146 mmol) in Ac₂O (300 mL) was added a solution of HNO₃ (14.1 g, 146 mmol, 65%) in AcOH (75 mL) at 0° C. The reaction mixture was stirred at 0˜5° C. for 3 h before aq. HCl (20%) was added dropwise at 0° C. The resulting mixture was extracted with EtOAc (200 mL×3). The organic layer was washed with sat. aq. NaHCO₃ then brine, dried over anhydrous Na₂SO₄ and evaporated under vacuum to give 1-(4-methoxy-3-nitro-phenyl)-cyclopropanecarboxylic acid methyl ester (36.0 g, 98%), which was directly used in the next step. ¹H NMR (CDCl₃, 300 MHz) δ 7.84 (d, J=2.1 Hz, 1H), 7.54 (dd, J=2.1, 8.7 Hz, 1H), 7.05 (d, J=8.7 Hz, 1H), 3.97 (s, 3H), 3.65 (s, 3H), 1.68-1.64 (m, 2H), 1.22-1.18 (m, 2H).

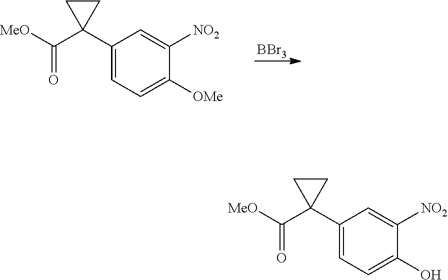

### 1-(4-Hydroxy-3-nitro-phenyl)-cyclopropanecarboxylic acid methyl ester

To a solution of 1-(4-methoxy-3-nitro-phenyl)-cyclopropane-carboxylic acid methyl ester (10.0 g, 39.8 mmol) in CH₂Cl₂ (100 mL) was added BBr₃ (12.0 g, 47.8 mmol) at −70° C. The mixture was stirred at −70° C. for 1 hour, then allowed to warm to −30° C. and stirred at this temperature for 3 hours. Water (50 mL) was added dropwise at −20° C., and the resulting mixture was allowed to warm room temperature before it was extracted with EtOAc (200 mL×3). The combined organic layers were dried over anhydrous Na₂SO₄ and evaporated under vacuum to give the crude product, which was purified by column chromatography on silica gel (petroleum ether/ethyl acetate 15:1) to afford 1-(4-hydroxy-3-nitro-phenyl)-cyclopropanecarboxylic acid methyl ester (8.3 g, 78%). ¹H NMR (CDCl₃,400 MHz) δ 10.5 (s, 1H), 8.05 (d, J=2.4 Hz, 1H), 7.59 (dd, J=2.0, 8.8 Hz, 1H), 7.11 (d, J=8.4 Hz, 1H), 3.64 (s, 3H), 1.68-1.64 (m, 2H), 1.20-1.15 (m, 2H).

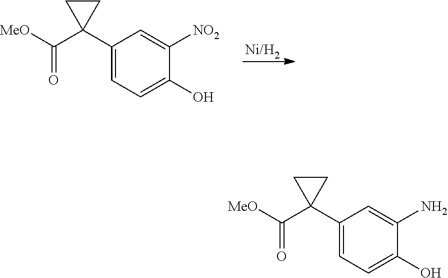 [SMILES: COC(=O)C1(c2ccc(O)c([N+](=O)[O-])c2)CC1 ; "Ni/H2" ; COC(=O)C1(c2ccc(O)c(N)c2)CC1]

### 1-(3-Amino-4-hydroxy-phenyl)-cyclopropanecarboxylic acid methyl ester

To a solution of 1-(4-hydroxy-3-nitro-phenyl)-cyclopropanecarboxylic acid methyl ester (8.3 g, 35 mmol) in MeOH (100 mL) was added Raney Nickel (0.8 g) under nitrogen atmosphere. The mixture was stirred under hydrogen atmosphere (1 atm) at 35° C. for 8 hours. The catalyst was filtered off through a Celite pad and the filtrate was evaporated under vacuum to give crude product, which was purified by column chromatography on silica gel (petroleum ether/ethyl acetate 1:1) to give 1-(3-amino-4-hydroxy-phenyl)-cyclopropanecarboxylic acid methyl ester (5.3 g, 74%). ¹H NMR (CDCl₃,400 MHz) δ 6.77 (s, 1H), 6.64 (d, J=2.0 Hz, 2H), 3.64 (s, 3H), 1.55-1.52 (m, 2H), 1.15-1.12 (m, 2H).

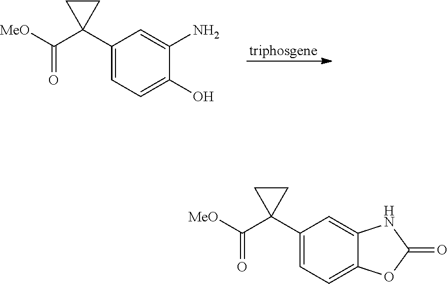 [SMILES: COC(=O)C1(c2ccc(O)c(N)c2)CC1 ; "triphosgene" ; COC(=O)C1(c2ccc3oc(=O)[nH]c3c2)CC1]

### 1-(2-Oxo-2,3-dihydro-benzooxazol-5-yl)-cyclopropanecarboxylic acid methyl ester

To a solution of 1-(3-amino-4-hydroxy-phenyl)-cyclopropanecarboxylic acid methyl ester (2.0 g, 9.6 mmol) in THF (40 mL) was added triphosgene (4.2 g, 14 mmol) at room temperature. The mixture was stirred for 20 minutes at this temperature before water (20 mL) was added dropwise at 0° C. The resulting mixture was extracted with EtOAc (100 mL×3). The combined organic layers were dried over anhydrous Na₂SO₄ and evaporated under vacuum to give 1-(2-oxo-2,3-dihydro-benzooxazol-5-yl)-cyclopropanecarboxylic acid methyl ester (2.0 g, 91%), which was directly used in the next step. ¹H NMR (CDCl₃,300 MHz) δ 8.66 (s, 1H), 7.13-7.12 (m, 2H), 7.07 (s, 1H), 3.66 (s, 3H), 1.68-1.65 (m, 2H), 1.24-1.20 (m, 2H).

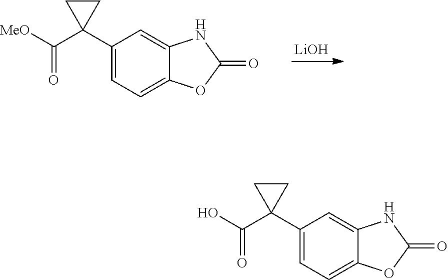

### 1-(2-Oxo-2,3-dihydrobenzo[d]oxazol-5-yl)cyclopropanecarboxylic acid

To a solution of 1-(2-oxo-2,3-dihydro-benzooxazol-5-yl)-cyclopropanecarboxylic acid methyl ester (1.9 g, 8.1 mmol) in MeOH (20 mL) and water (2 mL) was added LiOH.H₂O (1.7 g, 41 mmol) in portions at room temperature. The reaction mixture was stirred for 20 hours at 50° C. MeOH was removed by evaporation under vacuum before water (100 mL) and EtOAc (50 mL) were added. The aqueous layer was separated, acidified with HCl (3 mol/L) and extracted with EtOAc (100 mL×3). The combined organic layers were dried over anhydrous Na₂SO₄ and evaporated under vacuum to give 1-(2-oxo-2,3-dihydrobenzo[d]oxazol-5-yl)cyclopropanecarboxylic acid (1.5 g, 84%). ¹H NMR (DMSO, 400 MHz) δ 12.32 (brs, 1H), 11.59 (brs, 1H), 7.16 (d, J=8.4 Hz, 1H), 7.00 (d, J=8.0 Hz, 1H), 1.44-1.41 (m, 2H), 1.13-1.10 (m, 2H). MS (ESI) m/e (M+H+) 218.1.

### Example 6

### 1-(6-Fluoro-benzo[1,3]dioxol-5-yl)-cyclopropanecarboxylic acid

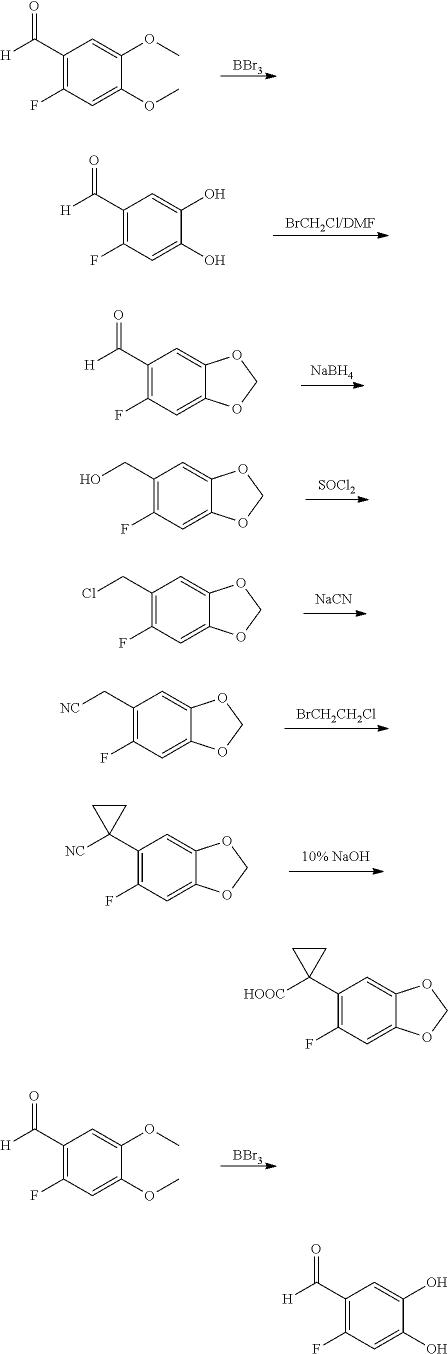

### 2-Fluoro-4,5-dihydroxy-benzaldehyde

To a stirred suspension of 2-fluoro-4,5-dimethoxy-benzaldehyde (3.00 g, 16.3 mmol) in dichloromethane (100 mL) was added BBr₃ (12.2 mL, 130 mmol) dropwise at −78° C. under nitrogen atmosphere. After addition, the mixture was warmed to −30° C. and stirred at this temperature for 5 h. The reaction mixture was poured into ice water and the precipitated solid was collected by filtration and washed with dichloromethane to afford 2-fluoro-4,5-dihydroxy-benzaldehyde (8.0 g), which was used directly in the next step.

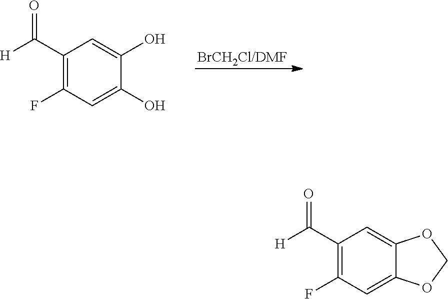 [SMILES: O=Cc1cc(O)c(O)cc1F ; "BrCH2Cl/DMF" ; O=Cc1cc2c(cc1F)OCO2]

6-Fluoro-benzo[1,3]dioxole-5-carbaldehyde To a stirred solution of 2-fluoro-4,5-dihydroxy-benzaldehyde (8.0 g) and BrClCH₂ (24.8 g, 190 mmol) in dry DMF (50 mL) was added Cs₂CO₃ (62.0 g, 190 mmol) in portions. The resulting mixture was stirred at 60° C. overnight and then poured into water. The mixture was extracted with EtOAc (200 mL×3). The combined organic layers were washed with brine (200 mL), dried over Na₂SO₄, and evaporated in vacuo to give crude product, which was purified by column chromatography on silica gel (5-20% ethyl acetate/petroleum ether) to afford 6-fluoro-benzo[1,3]dioxole-5-carbaldehyde (700 mg, two steps yield: 24%). ¹H-NMR (400 MHz, CDCl₃) δ 10.19 (s, 1H), 7.23 (d, J=5.6, 1H), 6.63 (d, J=9.6, 1H), 6.08 (s, 2H).

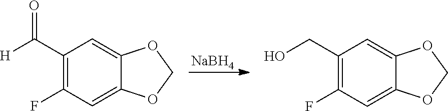

### (6-Fluoro-benzo[1,3]dioxol-5-yl)-methanol

To a stirred solution of 6-fluoro-benzo[1,3]dioxole-5-carbaldehyde (700 mg, 4.2 mmol) in MeOH (50 mL) was added NaBH₄ (320 mg, 8.4 mmol) in portions at 0° C. The mixture was stirred at this temperature for 30 min and was then concentrated in vacuo to give a residue. The residue was dissolved in EtOAc and the organic layer was washed with water, dried over Na₂SO₄, and concentrated in vacuo to afford (6-fluoro-benzo[1,3]dioxol-5-yl)-methanol (650 mg, 92%), which was directly used in the next step.

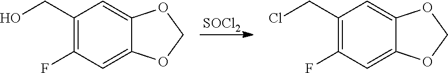

### 5-Chloromethyl-6-fluoro-benzo[1,3]dioxole

(6-Fluoro-benzo[1,3]dioxol-5-yl)-methanol (650 mg, 3.8 mmol) was added to SOCl₂ (20 mL) in portions at 0° C. The mixture was warmed to room temperature for 1 h and then heated at reflux for 1 h. The excess SOCl₂ was evaporated under reduced pressure to give the crude product, which was basified with sat. NaHCO₃ solution to pH ˜7. The aqueous phase was extracted with EtOAc (50 mL×3). The combined organic layers were dried over Na₂SO₄ and evaporated under reduced pressure to give 5-chloromethyl-6-fluoro-benzo[1,3]dioxole (640 mg, 90%), which was directly used in the next step.

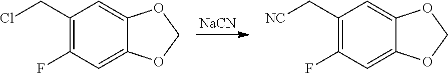

### (6-Fluoro-benzo[1,3]dioxol-5-yl)-acetonitrile

A mixture of 5-chloromethyl-6-fluoro-benzo[1,3]dioxole (640 mg, 3.4 mmol) and NaCN (340 mg, 6.8 mmol) in DMSO (20 mL) was stirred at 30° C. for 1 h and then poured into water. The mixture was extracted with EtOAc (50 mL×3). The combined organic layers were washed with water (50 mL) and brine (50 mL), dried over Na₂SO₄, and evaporated under reduced pressure to give the crude product, which was purified by column chromatography on silica gel (5-10% ethyl acetate/petroleum ether) to afford (6-fluoro-benzo[1,3]dioxol-5-yl)-acetonitrile (530 mg, 70%). ¹H-NMR (300 MHz, CDCl₃) δ 6.82 (d, J=4.8, 1 H), 6.62 (d, J=5.4, 1H), 5.99 (s, 2H), 3.65 (s, 2H).

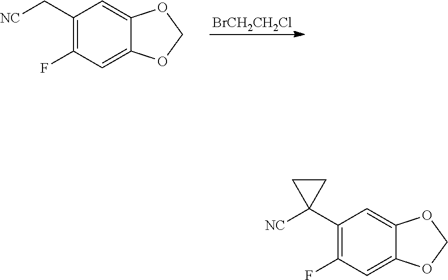

### 1-(6-Fluoro-benzo[1,3]dioxol-5-yl)-cyclopropanecarbonitrile

A flask was charged with water (10 mL), followed by a rapid addition of NaOH (10 g, 0.25 mol) in three portions over a 5 min period. The mixture was allowed to cool to room temperature. Subsequently, the flask was charged with toluene (6 mL), tetrabutyl-ammonium bromide (50 mg, 0.12 mmol), (6-fluoro-benzo[1,3]dioxol-5-yl)-acetonitrile (600 mg, 3.4 mmol) and 1-bromo-2-chloroethane (1.7 g, 12 mmol). The mixture stirred vigorously at 50° C. overnight. The cooled flask was charged with additional toluene (20 mL). The organic layer was separated and washed with water (30 mL) and brine (30 mL). The organic layer was removed in vacuo to give the crude product, which was purified by column chromatography on silica gel (5-10% ethyl acetate/petroleum ether) to give 1-(6-fluoro-benzo[1,3]dioxol-5-yl)-cyclopropanecarbonitrile (400 mg, 60%). ¹H NMR (300 MHz, CDCl₃) δ 6.73 (d, J=3.0 Hz, 1H), 6.61 (d, J=9.3 Hz, 1H), 5.98 (s, 2H), 1.67-1.62 (m, 2H), 1.31-1.27 (m, 2H).

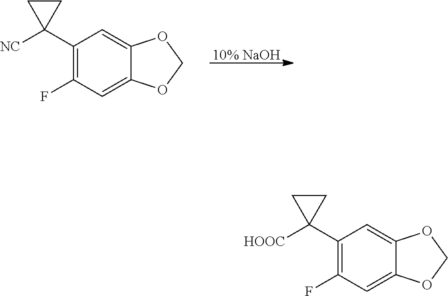

### 1-(6-Fluoro-benzo[1,3]dioxol-5-yl)-cyclopropanecarboxylic acid

A mixture of 1-(6-fluoro-benzo[1,3]dioxol-5-yl)-cyclopropanecarbonitrile (400 mg, 0.196 mmol) and 10% NaOH (10 mL) was stirred at 100° C. overnight. After the reaction was cooled, 5% HCl was added until the pH<5 and then EtOAc (30 mL) was added to the reaction mixture. The layers were separated and combined organic layers were evaporated in vacuo to afford 1-(6-fluoro-benzo[1,3]dioxol-5-yl)-cyclopropanecarboxylic acid (330 mg, 76%). ¹H NMR (400 MHz, DMSO) δ 12.2 (s, 1H), 6.87-6.85 (m, 2H), 6.00 (s, 1H), 1.42-1.40 (m, 2H), 1.14-1.07 (m, 2H).

### Example 7

### 1-(Benzofuran-5-yl)cyclopropanecarboxylic acid

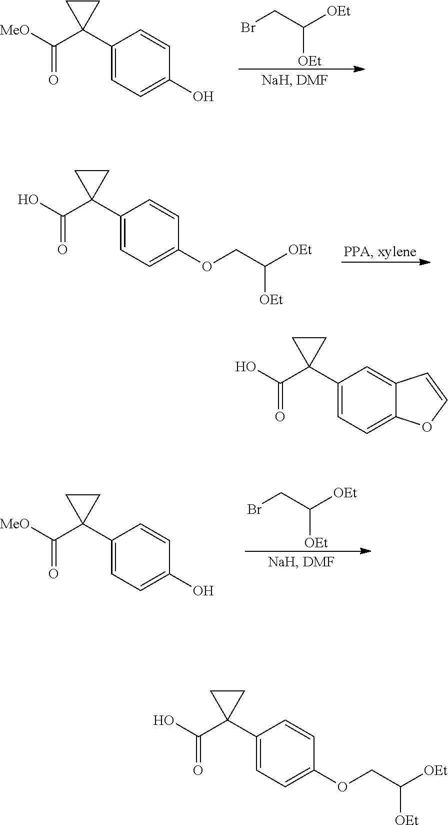

### 1-[4-(2,2-Diethoxy-ethoxy)-phenyl]-cyclopropanecarboxylic acid

To a stirred solution of 1-(4-hydroxy-phenyl)-cyclopropanecarboxylic acid methyl ester (15.0 g, 84.3 mmol) in DMF (50 mL) was added sodium hydride (6.7 g, 170 mmol, 60% in mineral oil) at 0° C. After hydrogen evolution ceased, 2-bromo-1,1-diethoxy-ethane (16.5 g, 84.3 mmol) was added dropwise to the reaction mixture. The reaction was stirred at 160° C. for 15 hours. The reaction mixture was poured onto ice (100 g) and was extracted with CH₂Cl₂. The combined organics were dried over Na₂SO₄. The solvent was evaporated under vacuum to give 1-[4-(2,2-diethoxy-ethoxy)phenyl]-cyclopropanecarboxylic acid (10 g), which was used directly in the next step without purification.

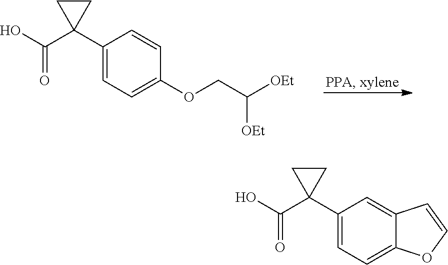 [SMILES: CCOC(COc1ccc(C2(C(=O)O)CC2)cc1)OCC ; "PPA, xylene" ; O=C(O)C1(c2ccc3occc3c2)CC1]

### 1-Benzofuran-5-yl-cyclopropanecarboxylic acid

To a suspension of 1-[4-(2,2-diethoxy-ethoxy)-phenyl]-cyclopropanecarboxylic acid (20 g, ˜65 mmol) in xylene (100 mL) was added PPA (22.2 g, 64.9 mmol) at room temperature. The mixture was heated at reflux (140° C.) for 1 hour before it was cooled to room temperature and decanted from the PPA. The solvent was evaporated under vacuum to obtain the crude product, which was purified by preparative HPLC to provide 1-(benzofuran-5-yl)cyclopropanecarboxylic acid (1.5 g, 5%). ¹H NMR (400 MHz, DMSO-d₆) δ 12.25 (br s, 1H), 7.95 (d, J=2.8 Hz, 1H), 7.56 (d, J=2.0 Hz, 1H), 7.47 (d, J=11.6 Hz, 1H), 7.25 (dd, J=2.4, 11.2 Hz, 1H), 6.89 (d, J=1.6 Hz, 1H), 1.47-1.44 (m, 2H), 1.17-1.14 (m, 2H).

### Example 8

### 1-(2,3-Dihydrobenzofuran-6-yl)cyclopropanecarboxylic acid

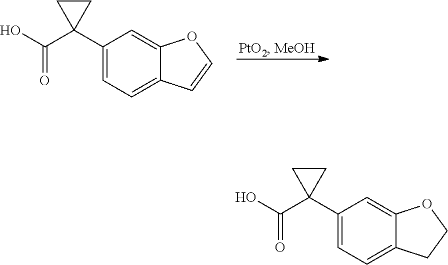

To a solution of 1-(benzofuran-6-yl)cyclopropanecarboxylic acid (370 mg, 1.8 mmol) in MeOH (50 mL) was added PtO₂ (75 mg, 20%) at room temperature. The reaction mixture was stirred under hydrogen atmosphere (1 atm) at 20° C. for 3 d. The reaction mixture was filtered and the solvent was evaporated in vacuo to afford the crude product, which was purified by prepared HPLC to give 1-(2,3-dihydrobenzofuran-6-yl)cyclopropanecarboxylic acid (155 mg, 42%). ¹H NMR (300 MHz, MeOD) δ 7.13 (d, J=7.5 Hz, 1H), 6.83 (d, J=7.8 Hz, 1H), 6.74 (s, 1H), 4.55 (t, J=8.7 Hz, 2H), 3.18 (t, J=8.7 Hz, 2H), 1.56-1.53 (m, 2H), 1.19-1.15 (m, 2H).

### Example 9

### 1-(3,3-Dimethyl-2,3-dihydrobenzofuran-5-yl)cyclopropanecarboxylic acid

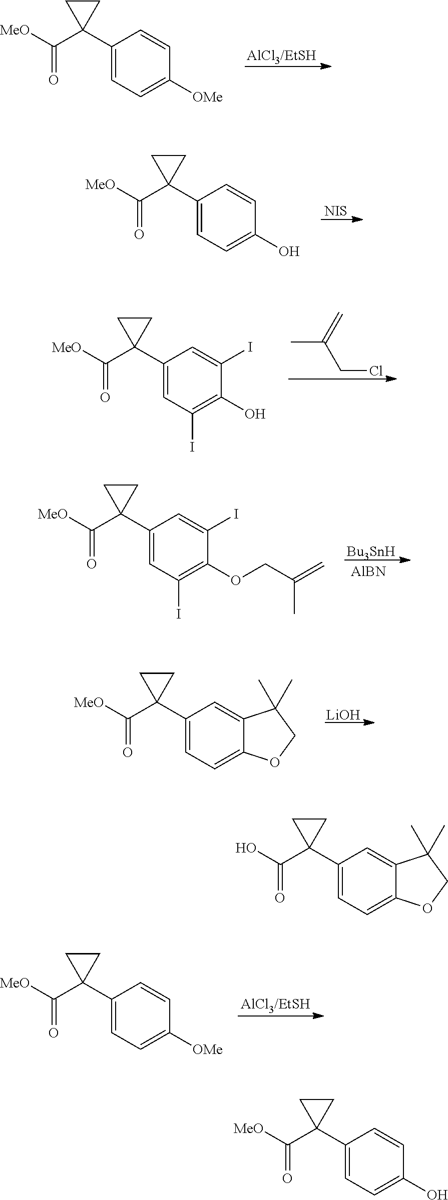

### 1-(4-Hydroxy-phenyl)-cyclopropanecarboxylic acid methyl ester

To a solution of methyl 1-(4-methoxyphenyl)cyclopropanecarboxylate (10.0 g, 48.5 mmol) in dichloromethane (80 mL) was added EtSH (16 mL) under ice-water bath. The mixture was stirred at 0° C. for 20 min before AlCl₃ (19.5 g, 0.15 mmol) was added slowly at 0° C. The mixture was stirred at 0° C. for 30 min. The reaction mixture was poured into ice-water, the organic layer was separated, and the aqueous phase was extracted with dichloromethane (50 mL×3). The combined organic layers were washed with H₂O, brine, dried over Na₂SO₄ and evaporated under vacuum to give 1-(4-hydroxy-phenyl)-cyclopropanecarboxylic acid methyl ester (8.9 g, 95%). ¹H NMR (400 MHz, CDCl₃) δ 7.20-7.17 (m, 2H), 6.75-6.72 (m, 2H), 5.56 (s, 1H), 3.63 (s, 3H), 1.60-1.57 (m, 2H), 1.17-1.15 (m, 2H).

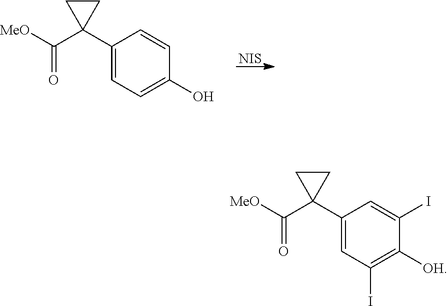

### 1-(4-Hydroxy-3,5-diiodo-phenyl)-cyclopropanecarboxylic acid methyl ester

To a solution of 1-(4-hydroxy-phenyl)-cyclopropanecarboxylic acid methyl ester (8.9 g, 46 mmol) in CH₃CN (80 mL) was added NIS (15.6 g, 69 mmol). The mixture was stirred at room temperature for 1 hour. The reaction mixture was concentrated and the residue was purified by column chromatography on silica gel (petroleum ether/ethyl acetate 10:1) to give 1-(4-hydroxy-3,5-diiodo-phenyl)-cyclopropanecarboxylic acid methyl ester (3.5 g, 18%). ¹H NMR (400 MHz, CDCl₃) δ 7.65 (s, 2H), 5.71 (s, 1H), 3.63 (s, 3H), 1.59-1.56 (m, 2H), 1.15-1.12 (m, 2H).

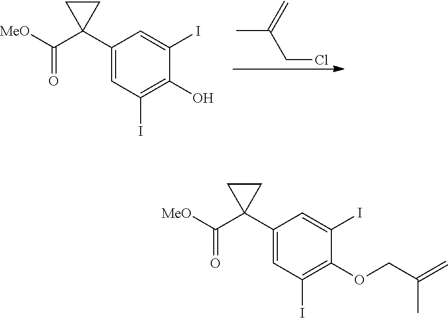

### 1-[3,5-Diiodo-4-(2-methyl-allyloxy)-phenyl]-cyclopropanecarboxylic acid methyl ester

A mixture of 1-(4-hydroxy-3,5-diiodo-phenyl)-cyclopropanecarboxylic acid methyl ester (3.2 g, 7.2 mmol), 3-chloro-2-methyl-propene (1.0 g, 11 mmol), K₂CO₃ (1.2 g, 8.6 mmol), NaI (0.1 g, 0.7 mmol) in acetone (20 mL) was stirred at 20° C. overnight. The solid was filtered off and the filtrate was concentrated under vacuum to give 1-[3,5-diiodo-4-(2-methyl-allyloxy)-phenyl]-cyclopropane-carboxylic acid methyl ester (3.5 g, 97%). ¹H NMR (300 MHz, CDCl₃) δ 7.75 (s, 2H), 5.26 (s, 1H), 5.06 (s, 1H), 4.38 (s, 2H), 3.65 (s, 3H), 1.98 (s, 3H), 1.62-1.58 (m, 2H), 1.18-1.15 (m, 2H).

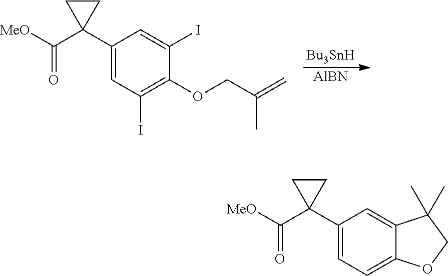

### 1-(3,3-Dimethyl-2,3-dihydro-benzofuran-5-yl)-cyclopropanecarboxylic acid methyl ester

To a solution of 1-[3,5-diiodo-4-(2-methyl-allyloxy)-phenyl]-cyclopropane-carboxylic acid methyl ester (3.5 g, 7.0 mmol) in toluene (15 mL) was added Bu₃SnH (2.4 g, 8.4 mmol) and AIBN (0.1 g, 0.7 mmol). The mixture was heated at reflux overnight. The reaction mixture was concentrated under vacuum and the residue was purified by column chromatography on silica gel (petroleum ether/ethyl acetate 20:1) to give 1-(3,3-dimethyl-2,3-dihydro-benzofuran-5-yl)-cyclopropanecarboxylic acid methyl ester (1.05 g, 62%). ¹H NMR (400 MHz, CDCl₃) δ 7.10-7.07 (m, 2H), 6.71 (d, J=8 Hz, 1H), 4.23 (s, 2H), 3.62 (s, 3H), 1.58-1.54 (m, 2H), 1.34 (s, 6H), 1.17-1.12 (m, 2H).

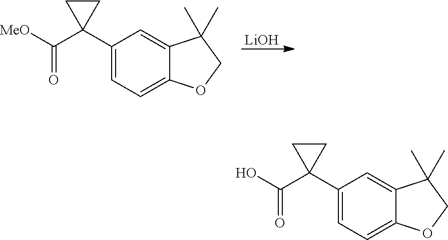

### 1-(3,3-Dimethyl-2,3-dihydrobenzofuran-5-yl)cyclopropanecarboxylic acid

To a solution of 1-(3,3-dimethyl-2,3-dihydro-benzofuran-5-yl)-cyclopropanecarboxylic acid methyl ester (1.0 g, 4.0 mmol) in MeOH (10 mL) was added LiOH (0.40 g, 9.5 mmol). The mixture was stirred at 40° C. overnight. HCl (10%) was added slowly to adjust the pH to 5. The resulting mixture was extracted with ethyl acetate (10 mL×3). The extracts were washed with brine and dried over Na₂SO₄. The solvent was removed under vacuum and the crude product was purified by preparative HPLC to give 1-(3,3-dimethyl-2,3-dihydrobenzofuran-5-yl)cyclopropanecarboxylic acid (0.37 g, 41%). ¹H NMR (400 MHz, CDCl₃) δ 7.11-7.07 (m, 2H), 6.71 (d, J=8 Hz, 1H), 4.23 (s, 2H), 1.66-1.63 (m, 2H), 1.32 (s, 6H), 1.26-1.23 (m, 2H).

### Example 10

### 2-(7-Methoxybenzo[d][1,3]dioxol-5-yl)acetonitrile

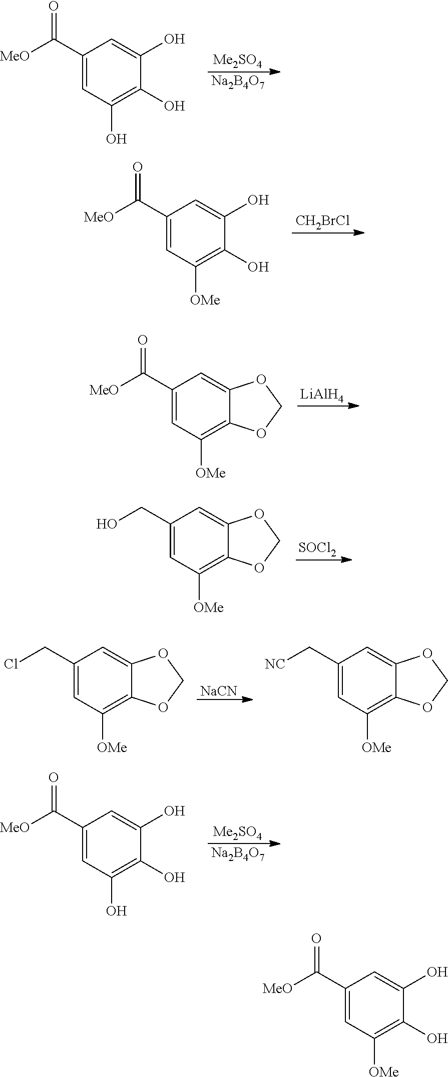

### 3,4-Dihydroxy-5-methoxybenzoate

To a solution of 3,4,5-trihydroxy-benzoic acid methyl ester (50 g, 0.27 mol) and Na₂B₄O₇ (50 g) in water (1000 mL) was added Me₂SO₄ (120 mL) and aqueous NaOH solution (25%, 200 mL) successively at room temperature. The mixture was stirred at room temperature for 6 h before it was cooled to 0° C. The mixture was acidified to pH ˜2 by adding conc. H₂SO₄ and then filtered. The filtrate was extracted with EtOAc (500 mL×3). The combined organic layers were dried over anhydrous Na₂SO₄ and evaporated under reduced pressure to give methyl 3,4-dihydroxy-5-methoxybenzoate (15.3 g 47%), which was used in the next step without further purification.

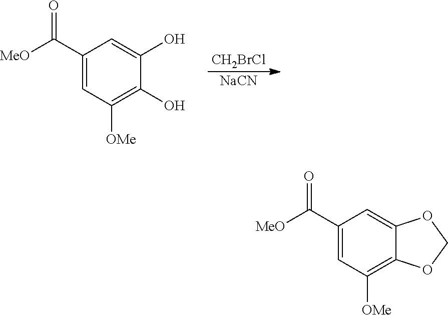

### Methyl 7-methoxybenzo[d][1,3]dioxole-5-carboxylate

To a solution of methyl 3,4-dihydroxy-5-methoxybenzoate (15.3 g, 0.0780 mol) in acetone (500 mL) was added CH₂BrCl (34.4 g, 0.270 mol) and K₂CO₃ (75.0 g, 0.540 mol) at 80° C. The resulting mixture was heated at reflux for 4 h. The mixture was cooled to room temperature and solid K₂CO₃ was filtered off. The filtrate was concentrated under reduced pressure, and the residue was dissolved in EtOAc (100 mL). The organic layer was washed with water, dried over anhydrous Na₂SO₄, and evaporated under reduced pressure to give the crude product, which was purified by column chromatography on silica gel (petroleum ether/ethyl acetate=10:1) to afford methyl 7-methoxybenzo[d][1,3]dioxole-5-carboxylate (12.6 g, 80%). ¹H NMR (400 MHz, CDCl₃) δ 7.32 (s, 1H), 7.21 (s, 1H), 6.05 (s, 2H), 3.93 (s, 3H), 3.88 (s, 3H).

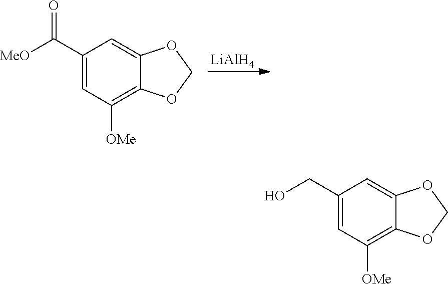

### (7-Methoxybenzo[d][1,3]dioxol-5-yl)methanol

To a solution of methyl 7-methoxybenzo[d][1,3]dioxole-5-carboxylate (14 g, 0.040 mol) in THF (100 mL) was added LiAlH₄ (3.1 g, 0.080 mol) in portions at room temperature. The mixture was stirred for 3 h at room temperature. The reaction mixture was cooled to 0° C. and treated with water (3.1 g) and NaOH (10%, 3.1 mL) successively. The slurry was filtered off and washed with THF. The combined filtrates were evaporated under reduced pressure to give (7-methoxy-benzo[d][1,3]dioxol-5-yl)methanol (7.2 g, 52%). ¹H NMR (400 MHz, CDCl₃) δ 6.55 (s, 1H), 6.54 (s, 1H), 5.96 (s, 2H), 4.57 (s, 2H), 3.90 (s, 3H).

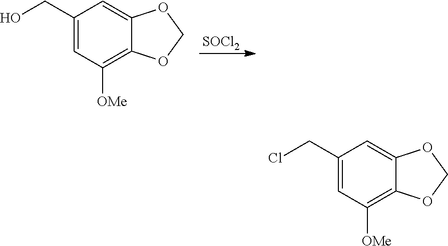

### 6-(Chloromethyl)-4-methoxybenzo[d][1,3]dioxole

To a solution of SOCl₂ (150 mL) was added (7-methoxybenzo[d][1,3]dioxol-5-yl)methanol (9.0 g, 54 mmol) in portions at 0° C. The mixture was stirred for 0.5 h. The excess SOCl₂ was evaporated under reduced pressure to give the crude product, which was basified with sat. aq. NaHCO₃ to pH ˜7. The aqueous phase was extracted with EtOAc (100 mL×3). The combined organic layers were dried over anhydrous Na₂SO₄ and evaporated to give 6-(chloromethyl)-4-methoxybenzo[d][1,3]dioxole (10 g 94%), which was used in the next step without further purification. ¹H NMR (400 MHz, CDCl₃) δ 6.58 (s, 1H), 6.57 (s, 1H), 5.98 (s, 2H), 4.51 (s, 2H), 3.90 (s, 3H).

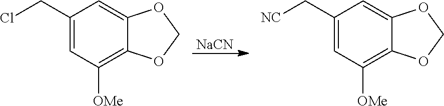

### 2-(7-Methoxybenzo[d][1,3]dioxol-5-yl)acetonitrile

To a solution of 6-(chloromethyl)-4-methoxybenzo[d][1,3]dioxole (10 g, 40 mmol) in DMSO (100 mL) was added NaCN (2.4 g, 50 mmol) at room temperature. The mixture was stirred for 3 h and poured into water (500 mL). The aqueous phase was extracted with EtOAc (100 mL×3). The combined organic layers were dried over anhydrous Na₂SO₄ and evaporated to give the crude product, which was washed with ether to afford 2-(7-methoxybenzo[d][1,3]dioxol-5-yl)acetonitrile (4.6 g, 45%). ¹H NMR (400 MHz, CDCl₃) δ 6.49 (s, 2H), 5.98 (s, 2H), 3.91 (s, 3H), 3.65 (s, 2H). ¹³C NMR (400 MHz, CDCl₃) δ 148.9, 143.4, 134.6, 123.4, 117.3, 107.2, 101.8, 101.3, 56.3, 23.1.

### Example 11

### 2-(3-(Benzyloxy)-4-methoxyphenyl)acetonitrile

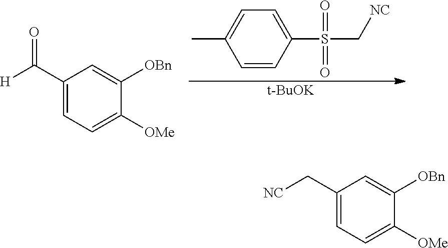

To a suspension of t-BuOK (20.2 g, 0.165 mol) in THF (250 mL) was added a solution of TosMIC (16.1 g, 82.6 mmol) in THF (100 mL) at −78° C. The mixture was stirred for 15 minutes, treated with a solution of 3-benzyloxy-4-methoxy-benzaldehyde (10.0 g, 51.9 mmol) in THF (50 mL) dropwise, and continued to stir for 1.5 hours at −78° C. To the cooled reaction mixture was added methanol (50 mL). The mixture was heated at reflux for 30 minutes. Solvent was removed to give a crude product, which was dissolved in water (300 mL). The aqueous phase was extracted with EtOAc (100 mL×3). The combined organic layers were dried and evaporated under reduced pressure to give crude product, which was purified by column chromatography (petroleum ether/ethyl acetate 10:1) to afford 2-(3-(benzyloxy)-4-methoxyphenyl)-acetonitrile (5.0 g, 48%). ¹H NMR (300 MHz, CDCl₃) δ 7.48-7.33 (m, 5H), 6.89-6.86 (m, 3H), 5.17 (s, 2H), 3.90 (s, 3H), 3.66 (s, 2H). ¹³C NMR (75 MHz, CDCl₃) δ 149.6, 148.6, 136.8, 128.8, 128.8, 128.2, 127.5, 127.5, 122.1, 120.9, 118.2, 113.8, 112.2, 71.2, 56.2, 23.3.

### Example 12

### 2-(3-(Benzyloxy)-4-chlorophenyl)acetonitrile

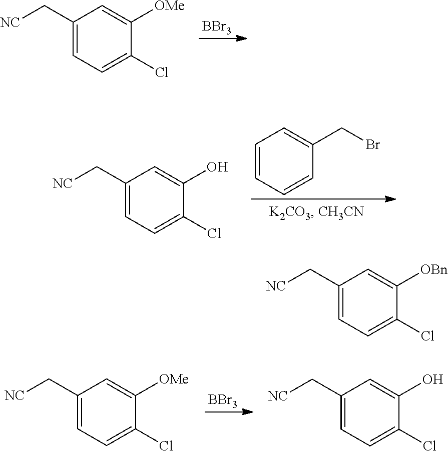

### (4-Chloro-3-hydroxy-phenyl)acetonitrile

BBr₃ (17 g, 66 mmol) was slowly added to a solution of 2-(4-chloro-3-methoxyphenyl)acetonitrile (12 g, 66 mmol) in dichloromethane (120 mL) at −78° C. under N₂. The reaction temperature was slowly increased to room temperature. The reaction mixture was stirred overnight and then poured into ice and water. The organic layer was separated, and the aqueous layer was extracted with dichloromethane (40 mL×3). The combined organic layers were washed with water, brine, dried over Na₂SO₄, and concentrated under vacuum to give (4-chloro-3-hydroxy-phenyl)-acetonitrile (9.3 g, 85%). ¹H NMR (300 MHz, CDCl₃) δ 7.34 (d, J=8.4 Hz, 1H), 7.02 (d, J=2.1 Hz, 1H), 6.87 (dd, J=2.1, 8.4 Hz, 1H), 5.15 (brs, 1H), 3.72 (s, 2H).

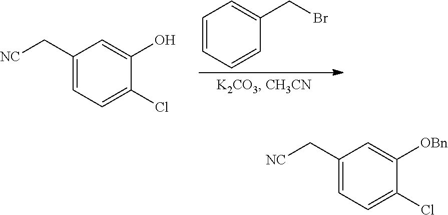

### 2-(3-(Benzyloxy)-4-chlorophenyl)acetonitrile

To a solution of (4-chloro-3-hydroxy-phenyl)acetonitrile (6.2 g, 37 mmol) in CH₃CN (80 mL) was added K₂CO₃ (10 g, 74 mmol) and BnBr (7.6 g, 44 mmol). The mixture was stirred at room temperature overnight. The solids were filtered off and the filtrate was evaporated under vacuum. The residue was purified by column chromatography on silica gel (petroleum ether/ethyl acetate 50:1) to give 2-(3-(benzyloxy)-4-chlorophenyl)-acetonitrile (5.6 g, 60%). ¹H NMR (400 MHz, CDCl₃) δ 7.48-7.32 (m, 6H), 6.94 (d, J=2 Hz, 2H), 6.86 (dd, J=2.0, 8.4 Hz, 1H), 5.18 (s, 2H), 3.71 (s, 2H).

### Example 13

### 2-(3-(Benzyloxy)-4-methoxyphenyl)acetonitrile

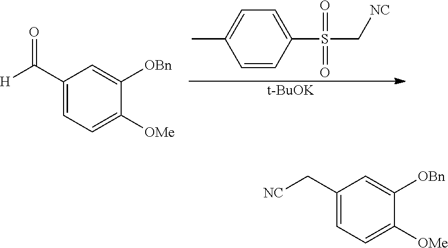

To a suspension of t-BuOK (20.2 g, 0.165 mol) in THF (250 mL) was added a solution of TosMIC (16.1 g, 82.6 mmol) in THF (100 mL) at −78° C. The mixture was stirred for 15 minutes, treated with a solution of 3-benzyloxy-4-methoxy-benzaldehyde (10.0 g, 51.9 mmol) in THF (50 mL) dropwise, and continued to stir for 1.5 hours at −78° C. To the cooled reaction mixture was added methanol (50 mL). The mixture was heated at reflux for 30 minutes. Solvent of the reaction mixture was removed to give a crude product, which was dissolved in water (300 mL). The aqueous phase was extracted with EtOAc (100 mL×3). The combined organic layers were dried and evaporated under reduced pressure to give crude product, which was purified by column chromatography (petroleum ether/ethyl acetate 10:1) to afford 2-(3-(benzyloxy)-4-methoxyphenyl)acetonitril (5.0 g, 48%). ¹H NMR (300 MHz, CDCl₃) δ 7.48-7.33 (m, 5H), 6.89-6.86 (m, 3H), 5.17 (s, 2H), 3.90 (s, 3H), 3.66 (s, 2H). ¹³C NMR (75 MHz, CDCl₃) δ 149.6, 148.6, 136.8, 128.8, 128.8, 128.2, 127.5, 127.5, 122.1, 120.9, 118.2, 113.8, 112.2, 71.2, 56.2, 23.3.

### Example 14

### 2-(3-Chloro-4-methoxyphenyl)acetonitrile

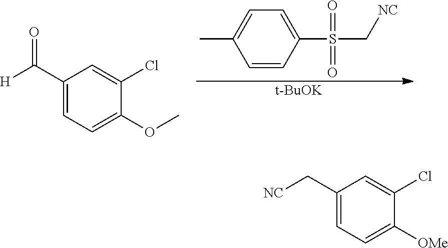

To a suspension of t-BuOK (4.8 g, 40 mmol) in THF (30 mL) was added a solution of TosMIC (3.9 g, 20 mmol) in THF (10 mL) at −78° C. The mixture was stirred for 10 minutes, treated with a solution of 3-chloro-4-methoxy-benzaldehyde (1.7 g, 10 mmol) in THF (10 mL) dropwise, and continued to stir for 1.5 hours at −78° C. To the cooled reaction mixture was added methanol (10 mL). The mixture was heated at reflux for 30 minutes. Solvent of the reaction mixture was removed to give a crude product, which was dissolved in water (20 mL). The aqueous phase was extracted with EtOAc (20 mL×3). The combined organic layers were dried and evaporated under reduced pressure to give crude product, which was purified by column chromatography (petroleum ether/ethyl acetate 10:1) to afford 2-(3-chloro-4-methoxyphenyl)acetonitrile (1.5 g, 83%). ¹H NMR (400 MHz, CDCl₃) δ 7.33 (d, J=2.4 Hz, 1H), 7.20 (dd, J=2.4, 8.4 Hz, 1H), 6.92 (d, J=8.4 Hz, 1H), 3.91 (s, 3H), 3.68 (s, 2H). ¹³C NMR (100 MHz, CDCl₃) δ 154.8, 129.8, 127.3, 123.0, 122.7, 117.60, 112.4, 56.2, 22.4.

### Example 15

### 2-(3-Fluoro-4-methoxyphenyl)acetonitrile

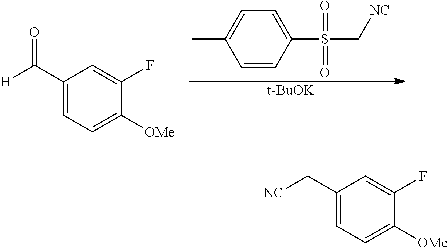

To a suspension of t-BuOK (25.3 g, 0.207 mol) in THF (150 mL) was added a solution of TosMIC (20.3 g, 0.104 mol) in THF (50 mL) at −78° C. The mixture was stirred for 15 minutes, treated with a solution of 3-fluoro-4-methoxy-benzaldehyde (8.00 g, 51.9 mmol) in THF (50 mL) dropwise, and continued to stir for 1.5 hours at −78° C. To the cooled reaction mixture was added methanol (50 mL). The mixture was heated at reflux for 30 minutes. Solvent of the reaction mixture was removed to give a crude product, which was dissolved in water (200 mL). The aqueous phase was extracted with EtOAc (100 mL×3). The combined organic layers were dried and evaporated under reduced pressure to give crude product, which was purified by column chromatography (petroleum ether/ethyl acetate 10:1) to afford 2-(3-fluoro-4-methoxyphenyl)acetonitrile (5.0 g, 58%). ¹H NMR (400 MHz, CDCl₃) δ 7.02-7.05 (m, 2H), 6.94 (t, J=8.4 Hz, 1H), 3.88 (s, 3H), 3.67 (s, 2H). ¹³C NMR (100 MHz, CDCl₃) δ 152.3, 147.5, 123.7, 122.5, 117.7, 115.8, 113.8, 56.3, 22.6.

### Example 16

### 2-(4-Chloro-3-methoxyphenyl)acetonitrile

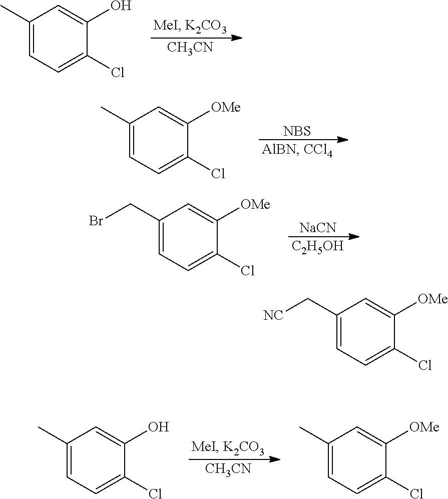

### Chloro-2-methoxy-4-methyl-benzene

To a solution of 2-chloro-5-methyl-phenol (93 g, 0.65 mol) in CH₃CN (700 mL) was added CH₃I (110 g, 0.78 mol) and K₂CO₃ (180 g, 1.3 mol). The mixture was stirred at 25° C. overnight. The solid was filtered off and the filtrate was evaporated under vacuum to give 1-chloro-2-methoxy-4-methyl-benzene (90 g, 89%). ¹H NMR (300 MHz, CDCl₃) δ 7.22 (d, J=7.8 Hz, 1H), 6.74-6.69 (m, 2H), 3.88 (s, 3H), 2.33 (s, 3H).

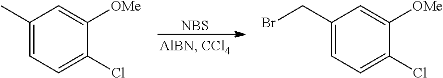

### 4-Bromomethyl-1-chloro-2-methoxy-benzene

To a solution of 1-chloro-2-methoxy-4-methyl-benzene (50 g, 0.32 mol) in CCl₄ (350 mL) was added NBS (57 g, 0.32 mol) and AIBN (10 g, 60 mmol). The mixture was heated at reflux for 3 hours. The solvent was evaporated under vacuum and the residue was purified by column chromatography on silica gel (petroleum ether/ethyl acetate=20:1) to give 4-bromomethyl-1-chloro-2-methoxy-benzene (69 g, 92%). ¹H NMR (400 MHz, CDCl₃) δ 7.33-7.31 (m, 1H), 6.95-6.91 (m, 2H), 4.46 (s, 2H), 3.92 (s, 3H).

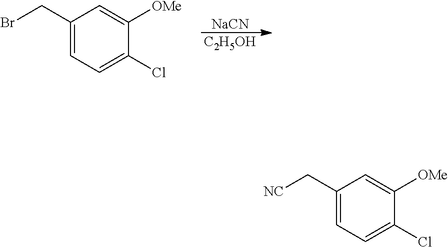

### 2-(4-Chloro-3-methoxyphenyl)acetonitrile

To a solution of 4-bromomethyl-1-chloro-2-methoxy-benzene (68.5 g, 0.290 mol) in C₂H₅OH (90%, 500 mL) was added NaCN (28.5 g, 0.580 mol). The mixture was stirred at 60° C. overnight. Ethanol was evaporated and the residue was dissolved in H₂O. The mixture was extracted with ethyl acetate (300 mL×3). The combined organic layers were washed with brine, dried over Na₂SO₄ and purified by column chromatography on silica gel (petroleum ether/ethyl acetate 30:1) to give 2-(4-chloro-3-methoxyphenyl)acetonitrile (25 g, 48%). ¹H NMR (400 MHz, CDCl₃) δ 7.36 (d, J=8 Hz, 1H), 6.88-6.84 (m, 2H), 3.92 (s, 3H), 3.74 (s, 2H). ¹³C NMR (100 MHz, CDCl₃) δ 155.4, 130.8, 129.7, 122.4, 120.7, 117.5, 111.5, 56.2, 23.5.

### Example 17

### 1-(3-(Hydroxymethyl)-4-methoxyphenyl)cyclopropanecarboxylic acid

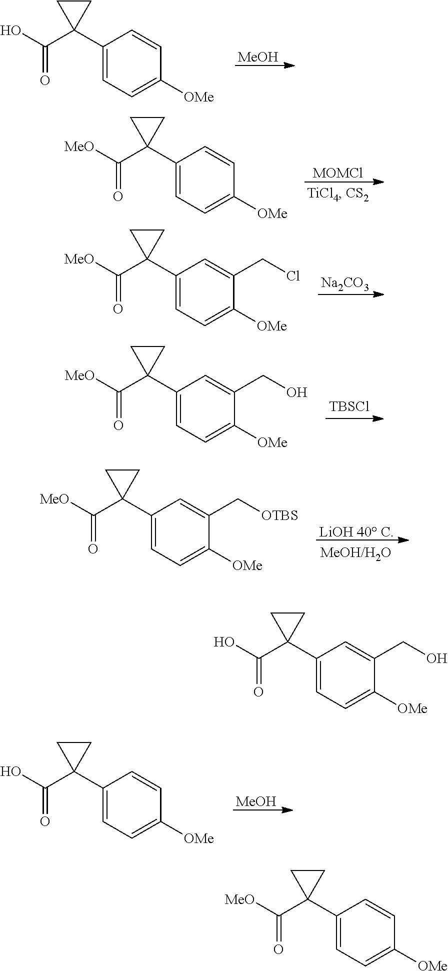

### 1-(4-Methoxy-phenyl)-cyclopropanecarboxylic acid methyl ester

To a solution of 1-(4-methoxy-phenyl)-cyclopropanecarboxylic acid (50 g, 0.26 mol) in MeOH (500 mL) was added toluene-4-sulfonic acid monohydrate (2.5 g, 13 mmol) at room temperature. The reaction mixture was heated at reflux for 20 hours. MeOH was removed by evaporation under vacuum and EtOAc (200 mL) was added. The organic layer was washed with sat. aq. NaHCO₃ (100 mL) and brine, dried over anhydrous Na₂SO₄ and evaporated under vacuum to give 1-(4-methoxy-phenyl)-cyclopropanecarboxylic acid methyl ester (53 g, 99%). ¹H NMR (CDCl₃, 400 MHz) δ 7.25-7.27 (m, 2H), 6.85 (d, J=8.8 Hz, 2H), 3.80 (s, 3H), 3.62 (s, 3H), 1.58 (m, 2H), 1.15 (m, 2H).

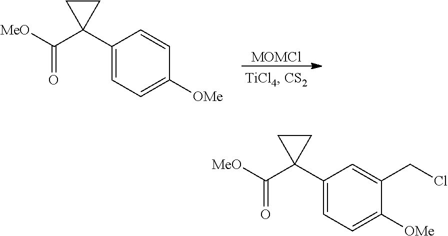 [SMILES: COC(=O)C1(c2ccc(OC)cc2)CC1 ; "MOMCl" ; COC(=O)C1(c2ccc(OC)c(CCl)c2)CC1]

### 1-(3-Chloromethyl-4-methoxy-phenyl)-cyclopropanecarboxylic acid methyl ester

To a solution of 1-(4-methoxy-phenyl)-cyclopropanecarboxylic acid methyl ester (30.0 g, 146 mmol) and MOMCl (29.1 g, 364 mmol) in CS₂ (300 mL) was added TiCl₄ (8.30 g, 43.5 mmol) at 5° C. The reaction mixture was heated at 30° C. for 1 d and poured into ice-water. The mixture was extracted with CH₂Cl₂ (150 mL×3). The combined organic extracts were evaporated under vacuum to give 1-(3-chloromethyl-4-methoxy-phenyl)-cyclopropanecarboxylic acid methyl ester (38.0 g), which was used in the next step without further purification.

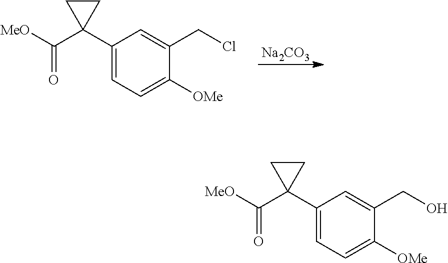

### 1-(3-Hydroxymethyl-4-methoxy-phenyl)-cyclopropanecarboxylic acid methyl ester

To a suspension of 1-(3-chloromethyl-4-methoxy-phenyl)-cyclopropanecarboxylic acid methyl ester (20 g) in water (350 mL) was added Bu₄NBr (4.0 g) and Na₂CO₃ (90 g, 0.85 mol) at room temperature. The reaction mixture was heated at 65° C. overnight. The resulting solution was acidified with aq. HCl (2 mol/L) and extracted with EtOAc (200 mL×3). The organic layer was washed with brine, dried over anhydrous Na₂SO₄ and evaporated under vacuum to give crude product, which was purified by column (petroleum ether/ethyl acetate 15:1) to give 1-(3-hydroxymethyl-4-methoxy-phenyl)-cyclopropanecarboxylic acid methyl ester (8.0 g, 39%). ¹H NMR (CDCl₃,400 MHz) δ 7.23-7.26 (m, 2H), 6.83 (d, J=8.0 Hz, 1H), 4.67 (s, 2H), 3.86 (s, 3H), 3.62 (s, 3H), 1.58 (q, J=3.6 Hz, 2 μl), 1.14-1.17 (m, 2H).

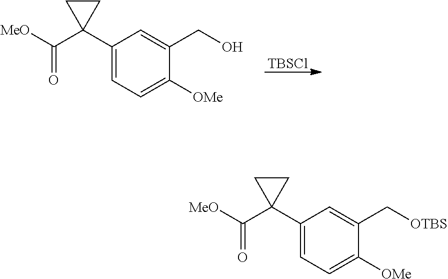

### 1-[3-(tert-Butyl-dimethyl-silanyloxymethyl)-4-methoxy-phenyl]cyclopropane carboxylic acid methyl ester

To a solution of 1-(3-hydroxymethyl-4-methoxy-phenyl)-cyclopropanecarboxylic acid methyl ester (8.0 g, 34 mmol) in CH₂Cl₂ (100 mL) were added imidazole (5.8 g, 85 mmol) and TBSCl (7.6 g, 51 mmol) at room temperature. The mixture was stirred overnight at room temperature. The mixture was washed with brine, dried over anhydrous Na₂SO₄ and evaporated under vacuum to give crude product, which was purified by column (petroleum ether/ethyl acetate 30:1) to give 1-[3-(tert-butyl-dimethyl-silanyloxymethyl)-4-methoxy-phenyl]-cyclopropanecarboxylic acid methyl ester (6.7 g, 56%). ¹H NMR (CDCl₃,400 MHz) δ 7.44-7.45 (m, 1H), 7.19 (dd, J=2.0, 8.4 Hz, 1H), 6.76 (d, J=8.4 Hz, 1H), 4.75 (s, 2H), 3.81 (s, 3H), 3.62 (s, 3H), 1.57-1.60 (m, 2H), 1.15-1.18 (m, 2H), 0.96 (s, 9H), 0.11 (s, 6H).

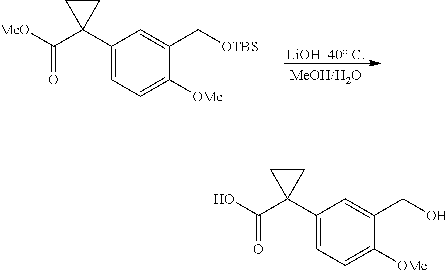 [SMILES: COC(=O)C1(c2ccc(OC)c(CO[Si](C)(C)C(C)(C)C)c2)CC1 ; "LiOH  40° C." ; COc1ccc(C2(C(=O)O)CC2)cc1CO]

### 1-(3-Hydroxymethyl-4-methoxy-phenyl)-cyclopropanecarboxylic acid

To a solution of 1-[3-(tert-butyl-dimethyl-silanyloxymethyl)-4-methoxy-phenyl]-cyclopropane carboxylic acid methyl ester (6.2 g, 18 mmol) in MeOH (75 mL) was added a solution of LiOH.H₂O (1.5 g, 36 mmol) in water (10 mL) at 0° C. The reaction mixture was stirred overnight at 40° C. MeOH was removed by evaporation under vacuum. AcOH (1 mol/L, 40 mL) and EtOAc (200 mL) were added. The organic layer was separated, washed with brine, dried over anhydrous Na₂SO₄ and evaporated under vacuum to provide 1-(3-hydroxymethyl-4-methoxy-phenyl)-cyclopropanecarboxylic acid (5.3 g).

### Example 18

### 2-(7-Chlorobenzo[d][1,3]dioxol-5-yl)acetonitrile

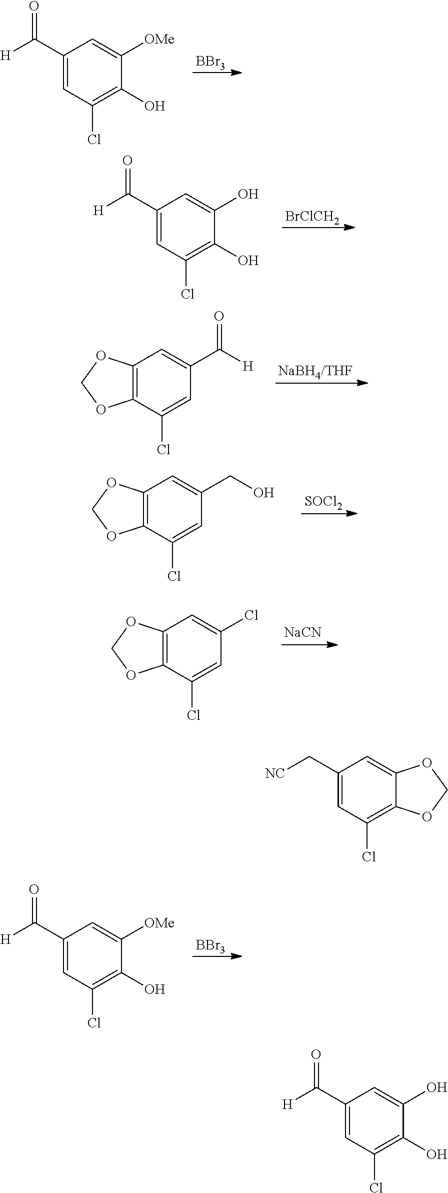

### 3-Chloro-4,5-dihydroxybenzaldehyde

To a suspension of 3-chloro-4-hydroxy-5-methoxy-benzaldehyde (10 g, 54 mmol) in dichloromethane (300 mL) was added BBr₃ (26.7 g, 107 mmol) dropwise at −40° C. under N₂. After addition, the mixture was stirred at this temperature for 5 h and then was poured into ice water. The precipitated solid was filtered and washed with petroleum ether. The filtrate was evaporated under reduced pressure to afford 3-chloro-4,5-dihydroxybenzaldehyde (9.8 g, 89%), which was directly used in the next step.

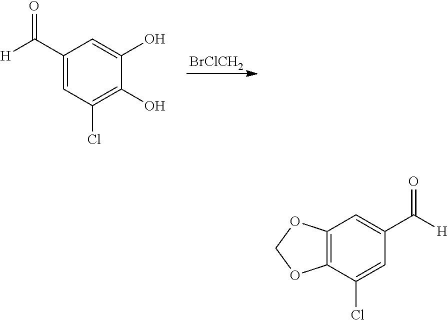

### 7-Chlorobenzo[d][1,3]dioxole-5-carbaldehyde

To a solution of 3-chloro-4,5-dihydroxybenzaldehyde (8.0 g, 46 mmol) and BrClCH₂ (23.9 g, 185 mmol) in dry DMF (100 mL) was added Cs₂CO₃ (25 g, 190 mmol). The mixture was stirred at 60° C. overnight and was then poured into water. The resulting mixture was extracted with EtOAc (50 mL×3). The combined extracts were washed with brine (100 mL), dried over Na₂SO₄ and concentrated under reduced pressure to afford 7-chlorobenzo[d][1,3]dioxole-5-carbaldehyde (6.0 g, 70%). ¹H NMR (400 MHz, CDCl₃) δ 9.74 (s, 1H), 7.42 (d, J=0.4 Hz, 1H), 7.26 (d, J=3.6 Hz, 1H), 6.15 (s, 2H).

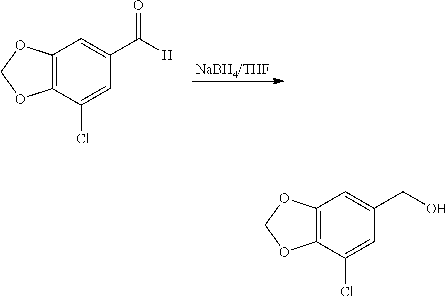

### (7-Chlorobenzo[d][1,3]dioxol-5-yl)methanol

To a solution of 7-chlorobenzo[d][1,3]dioxole-5-carbaldehyde (6.0 g, 33 mmol) in THF (50 mL) was added NaBH₄ (2.5 g, 64 mmol)) in portions at 0° C. The mixture was stirred at this temperature for 30 min and then poured into aqueous NH₄Cl solution. The organic layer was separated, and the aqueous phase was extracted with EtOAc (50 mL×3). The combined extracts were dried over Na₂SO₄ and evaporated under reduced pressure to afford (7-chlorobenzo[d][1,3]dioxol-5-yl)methanol, which was directly used in the next step.

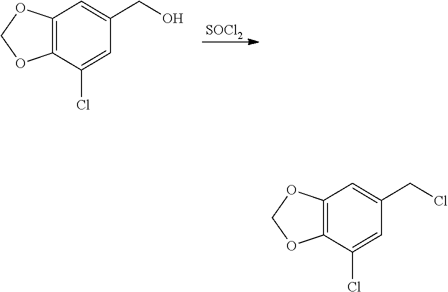

### 4-Chloro-6-(chloromethyl)benzo[d][1,3]dioxole

A mixture of (7-chlorobenzo[d][1,3]-dioxol-5-yl)methanol (5.5 g, 30 mmol) and SOCl₂ (5.0 mL, 67 mmol) in dichloromethane (20 mL) was stirred at room temperature for 1 h and was then poured into ice water. The organic layer was separated and the aqueous phase was extracted with dichloromethane (50 mL×3). The combined extracts were washed with water and aqueous NaHCO₃ solution, dried over Na₂SO₄ and evaporated under reduced pressure to afford 4-chloro-6-(chloromethyl)benzo[d][1,3]dioxole, which was directly used in the next step.

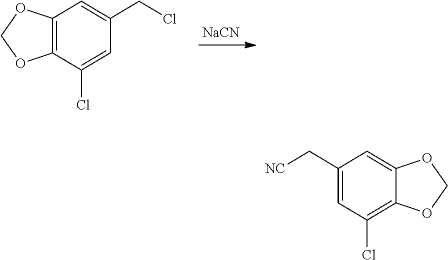

### 2-(7-Chlorobenzo[d][1,3]dioxol-5-yl)acetonitrile

A mixture of 4-chloro-6-(chloromethyl)benzo[d][1,3]dioxole (6.0 g, 29 mmol) and NaCN (1.6 g, 32 mmol) in DMSO (20 mL) was stirred at 40° C. for 1 h and was then poured into water. The mixture was extracted with EtOAc (30 mL three times). The combined organic layers were washed with water and brine, dried over Na₂SO₄ and evaporated under reduced pressure to afford 2-(7-chlorobenzo[d][1,3]dioxol-5-yl)acetonitrile (3.4 g, 58%). ¹H NMR δ 6.81 (s, 1H), 6.71 (s, 1H), 6.07 (s, 2H), 3.64 (s, 2H). ¹³C-NMR 8149.2, 144.3, 124.4, 122.0, 117.4, 114.3, 107.0, 102.3, 23.1.

### Example 19

### 1-(Benzo[d]oxazol-5-yl)cyclopropanecarboxylic acid

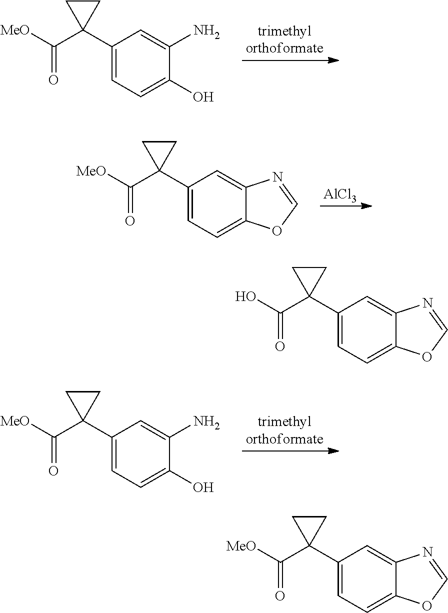

### 1-Benzooxazol-5-yl-cyclopropanecarboxylic acid methyl ester

To a solution of 1-(3-amino-4-hydroxyphenyl)cyclopropanecarboxylic acid methyl ester (3.00 g, 14.5 mmol) in DMF were added trimethyl orthoformate (5.30 g, 14.5 mmol) and a catalytic amount of p-toluenesulfonic acid monohydrate (0.3 g) at room temperature. The mixture was stirred for 3 hours at room temperature. The mixture was diluted with water and extracted with EtOAc (100 mL×3). The combined organic layers were dried over anhydrous Na₂SO₄ and evaporated under vacuum to give 1-benzooxazol-5-yl-cyclopropanecarboxylic acid methyl ester (3.1 g), which was directly used in the next step. ¹H NMR (CDCl₃,400 MHz) δ 8.09 (s, 1), 7.75 (d, J=1.2 Hz, 1H), 7.53-7.51 (m, 1H), 7.42-7.40 (m, 1H), 3.66 (s, 3H), 1.69-1.67 (m, 2H), 1.27-1.24 (m, 2H).

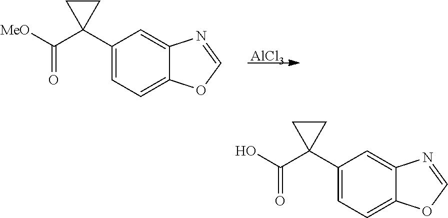

### 1-(Benzo[d]oxazol-5-yl)cyclopropanecarboxylic acid

To a solution of 1-benzooxazol-5-yl-cyclopropanecarboxylic acid methyl ester (2.9 g) in EtSH (30 mL) was added AlCl₃ (5.3 g, 40 mmol) in portions at 0° C. The reaction mixture was stirred for 18 hours at room temperature. Water (20 mL) was added dropwise at 0° C. The resulting mixture was extracted with EtOAc (100 mL three times). The combined organic layers were dried over anhydrous Na₂SO₄ and evaporated under vacuum to give the crude product, which was purified by column chromatography on silica gel (petroleum ether/ethyl acetate 1:2) to give 1-(benzo[d]oxazol-5-yl)cyclopropanecarboxylic acid (280 mg, 11% over two steps). ¹H NMR (DMSO, 400 MHz) δ 12.25 (brs, 1H), 8.71 (s, 1H), 7.70-7.64 (m, 2H), 7.40 (dd, J=1.6, 8.4 Hz, 1H), 1.49-1.46 (m, 2H), 1.21-1.18 (m, 2H). MS (ESI) m/e (M+H⁺) 204.4.

### Example 20

### 2-(7-Fluorobenzo[d][1,3]dioxol-5-yl)acetonitrile

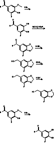

### 3-Fluoro-4,5-dihydroxy-benzaldehyde

To a suspension of 3-fluoro-4-hydroxy-5-methoxy-benzaldehyde (1.35 g, 7.94 mmol) in dichloromethane (100 mL) was added BBr₃ (1.5 mL, 16 mmol) dropwise at −78° C. under N₂. After addition, the mixture was warmed to −30° C. and it was stirred at this temperature for 5 h. The reaction mixture was poured into ice water. The precipitated solid was collected by filtration and washed with dichloromethane to afford 3-fluoro-4,5-dihydroxy-benzaldehyde (1.1 g, 89%), which was directly used in the next step.

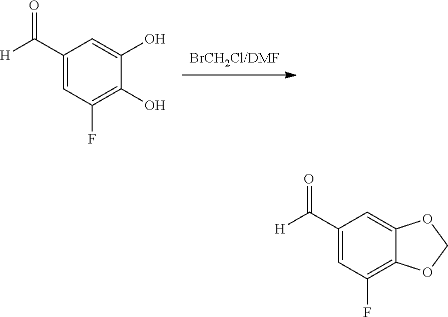 [SMILES: O=Cc1cc(O)c(O)c(F)c1 ; O=Cc1cc(F)c2c(c1)OCO2]

### 7-Fluoro-benzo[1,3]dioxole-5-carbaldehyde

To a solution of 3-fluoro-4,5-dihydroxy-benzaldehyde (1.5 g, 9.6 mmol) and BrClCH₂(4.9 g, 38.5 mmol) in dry DMF (50 mL) was added Cs₂CO₃ (12.6 g, 39 mmol). The mixture was stirred at 60° C. overnight and was then poured into water. The resulting mixture was extracted with EtOAc (50 mL×3). The combined organic layers were washed with brine (100 mL), dried over Na₂SO₄ and evaporated under reduced pressure to give the crude product, which was purified by column chromatography on silica gel (petroleum ether/ethyl acetate=10/1) to afford 7-fluoro-benzo[1,3]dioxole-5-carbaldehyde (0.80 g, 49%). ¹H NMR (300 MHz, CDCl₃) δ 9.78 (d, J=0.9 Hz, 1H), 7.26 (dd, J=1.5, 9.3 Hz, 1H), 7.19 (d, J=1.2 Hz, 1H), 6.16 (s, 2H).

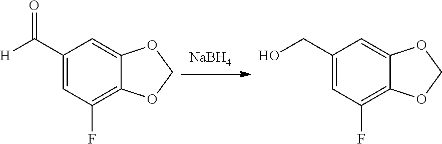

### (7-Fluoro-benzo[1,3]dioxol-5-yl)-methanol

To a solution of 7-fluoro-benzo[1,3]dioxole-5-carbaldehyde (0.80 g, 4.7 mmol) in MeOH (50 mL) was added NaBH₄ (0.36 g, 9.4 mmol) in portions at 0° C. The mixture was stirred at this temperature for 30 min and was then concentrated to dryness. The residue was dissolved in EtOAc. The EtOAc layer was washed with water, dried over Na₂SO₄ and concentrated to dryness to afford (7-fluoro-benzo[1,3]dioxol-5-yl)-methanol (0.80 g, 98%), which was directly used in the next step.

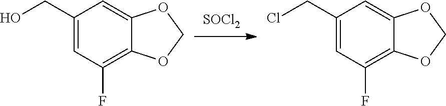

### 6-Chloromethyl-4-fluoro-benzo[1,3]dioxole

To SOCl₂ (20 mL) was added (7-fluoro-benzo[1,3]dioxol-5-yl)-methanol (0.80 g, 4.7 mmol) in portions at 0° C. The mixture was warmed to room temperature over 1 h and then was heated at reflux for 1 h. The excess SOCl₂ was evaporated under reduced pressure to give the crude product, which was basified with saturated aqueous NaHCO₃ to pH ˜7. The aqueous phase was extracted with EtOAc (50 mL three times). The combined organic layers were dried over Na₂SO₄ and evaporated under reduced pressure to give 6-chloromethyl-4-fluoro-benzo[1,3]dioxole (0.80 g, 92%), which was directly used in the next step.

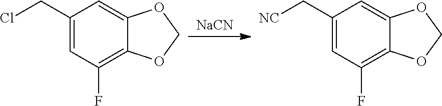

### 2-(7-Fluorobenzo[d][1,3]dioxol-5-yl)acetonitrile

A mixture of 6-chloromethyl-4-fluoro-benzo[1,3]dioxole (0.80 g, 4.3 mmol) and NaCN (417 mg, 8.51 mmol) in DMSO (20 mL) was stirred at 30° C. for 1 h and was then poured into water. The mixture was extracted with EtOAc (50 mL×3). The combined organic layers were washed with water (50 mL) and brine (50 mL), dried over Na₂SO₄ and evaporated under reduced pressure to give the crude product, which was purified by column chromatography on silica gel (petroleum ether/ethyl acetate=10/1) to afford 2-(7-fluorobenzo[d][1,3]dioxol-5-yl)acetonitrile (530 mg, 70%). ¹H NMR (300 MHz, CDCl₃) δ 6.68-6.64 (m, 2H), 6.05 (s, 2H), 3.65 (s, 2H). ¹³C-NMR 6151.1, 146.2, 134.1, 124.2, 117.5, 110.4, 104.8, 102.8, 23.3.

### Example 21

### 1-(1H-Indol-5-yl)cyclopropanecarboxylic acid

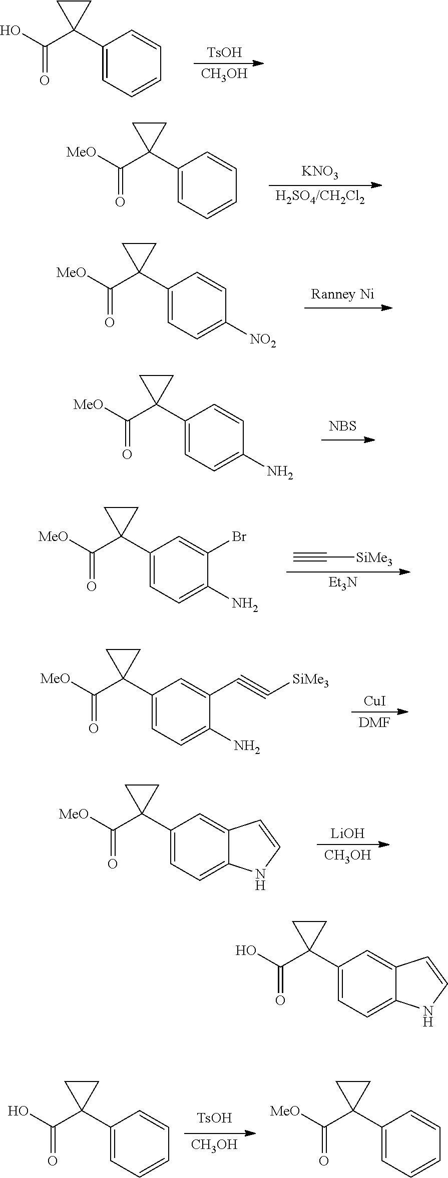

### Methyl 1-phenylcyclopropanecarboxylate

To a solution of 1-phenylcyclopropanecarboxylic acid (25 g, 0.15 mol) in CH₃OH (200 mL) was added TsOH (3 g, 0.1 mol) at room temperature. The mixture was refluxed overnight. The solvent was evaporated under reduced pressure to give crude product, which was dissolved into EtOAc. The EtOAc layer was washed with aq. sat. NaHCO₃. The organic layer was dried over anhydrous Na₂SO₄ and evaporated under reduced pressure to give methyl 1-phenylcyclopropanecarboxylate (26 g, 96%), which was used directly in the next step. ¹H NMR (400 MHz, CDCl₃) δ 7.37-7.26 (m, 5H), 3.63 (s, 3H), 1.63-1.60 (m, 2H), 1.22-1.19 (m, 2H).

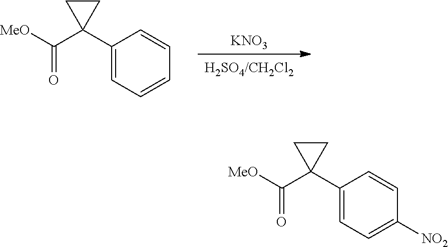

### Methyl 1-(4-nitrophenyl)cyclopropanecarboxylate

To a solution of 1-phenylcyclopropanecarboxylate (20.62 g, 0.14 mol) in H₂SO₄/CH₂Cl₂ (40 mL/40 mL) was added KNO₃(12.8 g, 0.13 mol) in portion at 0° C. The mixture was stirred for 0.5 hr at 0° C. Ice water was added and the mixture was extracted with EtOAc (100 mL×3). The organic layers were dried with anhydrous Na₂SO₄ and evaporated to give methyl 1-(4-nitrophenyl)cyclopropanecarboxylate (21 g, 68%), which was used directly in the next step. ¹H NMR (300 MHz, CDCl₃) δ 8.18 (dd, J=2.1, 6.9 Hz, 2H), 7.51 (dd, J=2.1, 6.9 Hz, 2H), 3.64 (s, 3H), 1.72-1.69 (m, 2H), 1.25-1.22 (m, 2H).

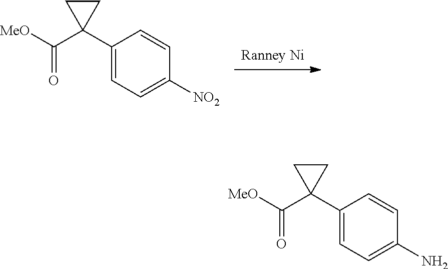 [SMILES: COC(=O)C1(c2ccc([N+](=O)[O-])cc2)CC1 ; COC(=O)C1(c2ccc(N)cc2)CC1]

### Methyl 1-(4-aminophenyl)cyclopropanecarboxylate

To a solution of methyl 1-(4-nitrophenyl)cyclopropanecarboxylate (20 g, 0.09 mol) in MeOH (400 mL) was added Ni (2 g) under nitrogen atmosphere. The mixture was stirred under hydrogen atmosphere (1 atm) at room temperature overnight. The catalyst was filtered off through a pad of Celite and the filtrate was evaporated under vacuum to give crude product, which was purified by chromatography column on silica gel (petroleum ether/ethyl acetate=10:1) to give methyl 1-(4-aminophenyl)cyclopropanecarboxylate (11.38 g, 66%). ¹H NMR (300 MHz, CDCl₃) δ 7.16 (d, J=8.1 Hz, 2H), 6.86 (d, J=7.8 Hz, 2H), 4.31 (br, 2H), 3.61 (s, 3H), 1.55-1.50 (m, 2H), 1.30-1.12 (m, 2H).

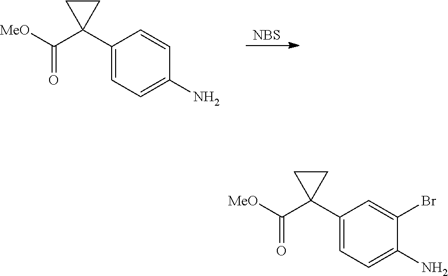 [SMILES: COC(=O)C1(c2ccc(N)cc2)CC1 ; "NBS" ; COC(=O)C1(c2ccc(N)c(Br)c2)CC1]

### Methyl 1-(4-amino-3-bromophenyl)cyclopropanecarboxylate

To a solution of methyl 1-(4-aminophenyl)cyclopropanecarboxylate (10.38 g, 0.05 mol) in acetonitrile (200 mL) was added NBS (9.3 g, 0.05 mol) at room temperature. The mixture was stirred overnight. Water (200 mL) was added. The organic layer was separated and the aqueous phase was extracted with EtOAc (80 mL×3). The organic layers were dried with anhydrous Na₂SO₄ and evaporated to give methyl 1-(4-amino-3-bromophenyl)cyclopropanecarboxylate (10.6 g, 78%), which was used directly in the next step. ¹H NMR (400 MHz, CDCl₃) δ 7.38 (d, J=2.0 Hz, 1H), 7.08 (dd, J=1.6, 8.4 Hz, 1H), 6.70 (d, J=8.4 Hz, 1H), 3.62 (s, 3H), 1.56-1.54 (m, 2H), 1.14-1.11 (m, 2H).

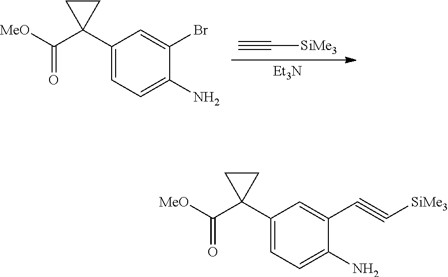

### Methyl 1-(4-amino-3-((trimethylsilyl)ethynyl)phenyl)cyclopropane carboxylate

To a degassed solution of methyl 1-(4-amino-3-bromophenyl)cyclopropane carboxylate (8 g, 0.03 mol) in Et₃N (100 mL) was added ethynyl-trimethyl-silane (30 g, 0.3 mol), DMAP (5% mol) and Pd(PPh₃)₂Cl₂ (5% mol) under N₂. The mixture was refluxed at 70° C. overnight. The insoluble solid was filtered off and washed with EtOAc (100 mL×3). The filtrate was evaporated under reduced pressure to give a residue, which was purified by chromatography column on silica gel (petroleum ether/ethyl acetate=20:1) to give methyl 1-(4-amino-3-((trimethylsilyl)ethynyl)phenyl)cyclopropanecarboxylate (4.8 g, 56%). ¹H NMR (300 MHz, CDCl₃) δ7.27 (s, 1H), 7.10 (dd, J=2.1, 8.4 Hz, 1H), 6.64 (d, J=8.4 Hz, 1H), 3.60 (s, 3H), 1.55-1.51 (m, 2H), 1.12-1.09 (m, 2H), 0.24 (s, 9H).

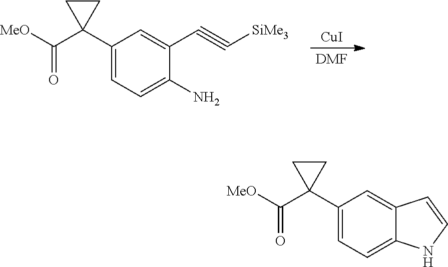

### Methyl 1-(1H-indol-5-yl)cyclopropanecarboxylate

To a degassed solution of methyl 1-(4-amino-3-((trimethylsilyl)ethynyl)phenyl)cyclopropanecarboxylate (4.69 g, 0.02 mol) in DMF (20 mL) was added CuI (1.5 g, 0.008 mol) under N₂ at room temperature. The mixture was stirred for 3 hr at room temperature. The insoluble solid was filtered off and washed with EtOAc (50 mL×3). The filtrate was evaporated under reduced pressure to give a residue, which was purified by chromatography column on silica gel (petroleum ether/ethyl acetate=20:1) to give methyl 1-(1H-indol-5-yl)cyclopropanecarboxylate (2.2 g, 51%). ¹H NMR (400 MHz, CDCl₃) δ 7.61 (s, 1H), 7.33 (d, J=8.4 Hz, 1H), 7.23-7.18 (m, 2 μl), 6.52-6.51 (m, 1H) 3.62 (s, 3H), 1.65-1.62 (m, 2H), 1.29-1.23 (m, 2H).

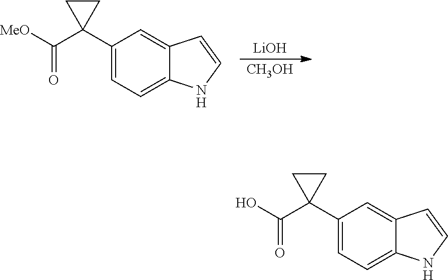

### 1-(1H-Indol-5-yl)cyclopropanecarboxylic acid

To a solution of methyl 1-(1H-indol-5-yl)cyclopropanecarboxylate (1.74 g, 8 mmol) in CH₃OH (50 mL) and water (20 mL) was added LiOH (1.7 g, 0.04 mol). The mixture was heated at 45° C. for 3 hr. Water was added and the mixture was acidified with concentrated HCl to pH ˜3 before being extracted with EtOAc (20 mL×3). The organic layers were dried over anhydrous Na₂SO₄ and evaporated to give 1-(1H-indol-5-yl)cyclopropanecarboxylic acid (1.4 g, 87%). ¹H NMR (300 MHz, DMSO-d₆) 7.43 (s, 1H), 7.30-7.26 (m, 2H), 7.04 (dd, J=1.5, 8.4 Hz, 1H), 6.35 (s, 1 μl), 1.45-1.41 (m, 2H), 1.14-1.10 (m, 2H).

### Example 22

### 1-(4-Oxochroman-6-yl)cyclopropanecarboxylic acid

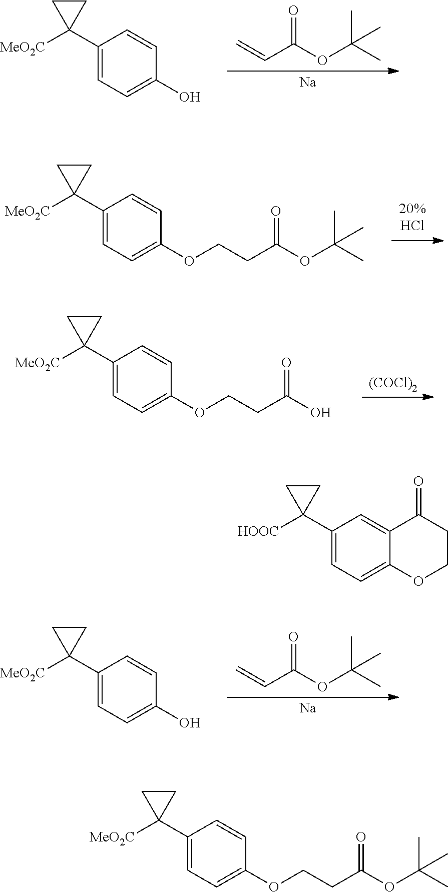

### 1-[4-(2-tert-Butoxycarbonyl-ethoxy)-phenyl]-cyclopropanecarboxylic methyl ester

To a solution of 1-(4-hydroxy-phenyl)-cyclopropanecarboxylic methyl ester (7.0 g, 3.6 mmol) in acrylic tert-butyl ester (50 mL) was added Na (42 mg, 1.8 mmol) at room temperature. The mixture was heated at 110° C. for 1 h. After cooling to room temperature, the resulting mixture was quenched with water and extracted with EtOAc (100 mL×3). The combined organic extracts were dried over anhydrous Na₂SO₄ and evaporated under vacuum to give the crude product, which was purified by column chromatography on silica gel (petroleum ether/ethyl acetate 20:1) to give 1-[4-(2-tert-butoxycarbonyl-ethoxy)-phenyl]-cyclopropanecarboxylic methyl ester (6.3 g, 54%) and unreacted start material (3.0 g). ¹H NMR (300 MHz, CDCl₃) δ 7.24 (d, J=8.7 Hz, 2H), 6.84 (d, J=8.7 Hz, 2H), 4.20 (t, J=6.6 Hz, 2H), 3.62 (s, 3H), 2.69 (t, J=6.6 Hz, 2H), 1.59-1.56 (m, 2H), 1.47 (s, 9H), 1.17-1.42 (m, 2H).

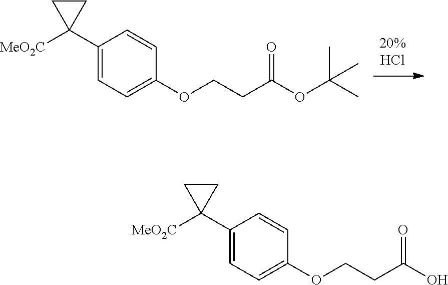 [SMILES: COC(=O)C1(c2ccc(OCCC(=O)OC(C)(C)C)cc2)CC1 ; COC(=O)C1(c2ccc(OCCC(=O)O)cc2)CC1]

### 1-[4-(2-Carboxy-ethoxy)-phenyl]-cyclopropanecarboxylic methyl ester

A solution of 1-[4-(2-tert-butoxycarbonyl-ethoxy)-phenyl]-cyclopropanecarboxylic methyl ester (6.3 g, 20 mmol) in HCl (20%, 200 mL) was heated at 110° C. for 1 h. After cooling to room temperature, the resulting mixture was filtered. The solid was washed with water and dried under vacuum to give 1-[4-(2-carboxy-ethoxy)-phenyl]-cyclopropanecarboxylic methyl ester (5.0 g, 96%). ¹H NMR (300 MHz, DMSO) δ 7.23-7.19 (m, 2H), 6.85-6.81 (m, 2H), 4.13 (t, J=6.0 Hz, 2H), 3.51 (s, 3H), 2.66 (t, J=6.0 Hz, 2H), 1.43-1.39 (m, 2H), 1.14-1.10 (m, 2H).

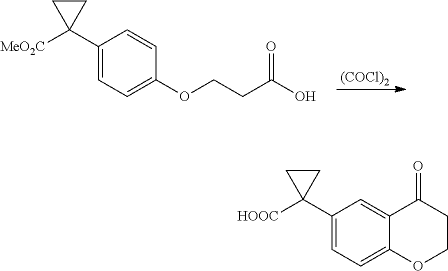 [SMILES: COC(=O)C1(c2ccc(OCCC(=O)O)cc2)CC1 ; "(COCl)2" ; O=C1CCOc2ccc(C3(C(=O)O)CC3)cc21]

### 1-(4-Oxochroman-6-yl)cyclopropanecarboxylic acid

To a solution of 1-[4-(2-carboxy-ethoxy)-phenyl]-cyclopropanecarboxylic methyl ester (5.0 g, 20 mmol) in CH₂Cl₂ (50 mL) were added oxalyl chloride (4.8 g, 38 mmol) and two drops of DMF at 0° C. The mixture was stirred at 0˜5° C. for 1 h and then evaporated under vacuum. To the resulting mixture was added CH₂Cl₂ (50 mL) at 0° C. and stirring was continued at 0-5° C. for 1 h. The reaction was slowly quenched with water and was extracted with EtOAc (50 mL×3). The combined organic extracts were dried over anhydrous Na₂SO₄ and evaporated under vacuum to give the crude product, which was purified by column chromatography on silica gel (petroleum ether/ethyl acetate 20:1-2:1) to give 1-(4-oxochroman-6-yl)cyclopropanecarboxylic acid (830 mg, 19%) and methyl 1-(4-oxochroman-6-yl)cyclopropanecarboxylate (1.8 g, 38%). 1-(4-Oxochroman-6-yl)cyclopropane-carboxylic acid: ¹H NMR (400 MHz, DMSO) δ 12.33 (br s, 1H), 7.62 (d, J=2.0 Hz, 1H), 7.50 (dd, J=2.4, 8.4 Hz, 1H), 6.95 (d, J=8.4 Hz, 1H), 4.50 (t, J=6.4 Hz, 2H), 2.75 (t, J=6.4 Hz, 2H), 1.44-1.38 (m, 2H), 1.10-1.07 (m, 2H). MS (ESI) m/z (M+H⁺) 231.4. 1-(4-Oxochroman-6-yl)cyclopropanecarboxylate: ¹H NMR (400 MHz, CDCl₃) δ 7.83 (d, J=2.4 Hz, 1H), 7.48 (dd, J=2.4, 8.4 Hz, 1H), 6.93 (d, J=8.4 Hz, 1H), 4.55-4.52 (m, 2H), 3.62 (s, 3H), 2.80 (t, J=6.4 Hz, 2H), 1.62-1.56 (m, 2H), 1.18-1.15 (m, 2H).

### Example 23

### 1-(4-Hydroxy-4-methoxychroman-6-yl)cyclopropanecarboxylic acid

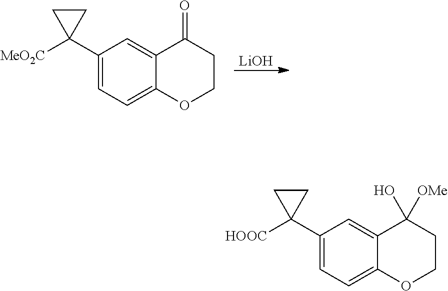

### 1-(4-Hydroxy-4-methoxychroman-6-yl)cyclopropanecarboxylic acid

To a solution of methyl 1-(4-oxochroman-6-yl)cyclopropanecarboxylate (1.0 g, 4.1 mmol) in MeOH (20 mL) and water (20 mL) was added LiOH.H₂O (0.70 g, 16 mmol) in portions at room temperature. The mixture was stirred overnight at room temperature before the MeOH was removed by evaporation under vacuum. Water and Et₂O were added to the residue and the aqueous layer was separated, acidified with HCl and extracted with EtOAc (50 mL×3). The combined organic extracts dried over anhydrous Na₂SO₄ and evaporated under vacuum to give 1-(4-hydroxy-4-methoxychroman-6-yl)cyclopropanecarboxylic acid (480 mg, 44%). ¹H NMR (400 MHz, CDCl₃) δ 12.16 (s, 1H), 7.73 (d, J=2.0 Hz, 1H), 7.47 (dd, J=2.0, 8.4 Hz, 1H), 6.93 (d, J=8.8 Hz, 1H), 3.83-3.80 (m, 2H), 3.39 (s, 3H), 3.28-3.25 (m, 2H), 1.71-1.68 (m, 2H), 1.25-1.22 (m, 2H). MS (ESI) m/z (M+H⁺⁾ 263.1.

### Example 24

### 1-(4-Hydroxy-4-methoxychroman-6-yl)cyclopropanecarboxylic acid

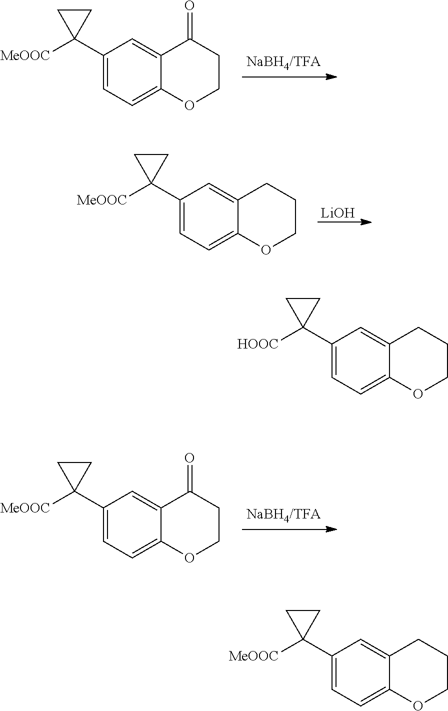

### 1-Chroman-6-yl-cyclopropanecarboxylic methyl ester

To trifluoroacetic acid (20 mL) was added NaBH₄ (0.70 g, 130 mmol) in portions at 0° C. under N₂ atmosphere. After stirring for 5 min, a solution of 1-(4-oxo-chroman-6-yl)-cyclopropanecarboxylic methyl ester (1.6 g, 6.5 mmol) was added at 15° C. The reaction mixture was stirred for 1 h at room temperature before being slowly quenched with water. The resulting mixture was extracted with EtOAc (50 mL×3). The combined organic extracts dried over anhydrous Na₂SO₄ and evaporated under vacuum to give 1-chroman-6-yl-cyclopropanecarboxylic methyl ester (1.4 g, 92%), which was used directly in the next step. ¹H NMR (300 MHz, CDCl₃) δ 7.07-7.00 (m, 2H), 6.73 (d, J=8.4 Hz, 1H), 4.17 (t, J=5.1 Hz, 2H), 3.62 (s, 3H), 2.79-2.75 (m, 2H), 2.05-1.96 (m, 2H), 1.57-1.54 (m, 2H), 1.16-1.13 (m, 2H).

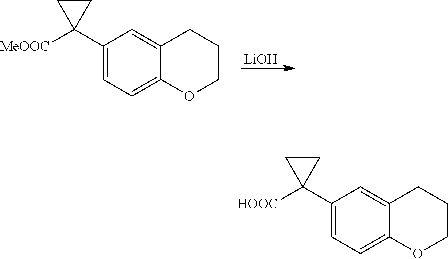

### 1-(4-Hydroxy-4-methoxychroman-6-yl)cyclopropanecarboxylic acid

To a solution of 1-chroman-6-yl-cyclopropanecarboxylic methyl ester (1.4 g, 60 mmol) in MeOH (20 mL) and water (20 mL) was added LiOH.H₂O (1.0 g, 240 mmol) in portions at room temperature. The mixture was stirred overnight at room temperature before the MeOH was removed by evaporation under vacuum. Water and Et₂O were added and the aqueous layer was separated, acidified with HCl and extracted with EtOAc (50 mL×3). The combined organic extracts dried over anhydrous Na₂SO₄ and evaporated under vacuum to give 1-(4-Hydroxy-4-methoxychroman-6-yl)cyclopropanecarboxylic acid (1.0 g, 76%). ¹H NMR (400 MHz, DMSO) δ 12.10 (br s, 1H), 6.95 (d, J=2.4 Hz, 2H), 6.61-6.59 (m, 1H), 4.09-4.06 (m, 2H), 2.70-2.67 (m, 2H), 1.88-1.86 (m, 2H), 1.37-1.35 (m, 2H), 1.04-1.01 (m, 2H). MS (ESI) m/z (M+H⁺) 217.4.

### Example 25

### 1-(3-Methylbenzo[d]isoxazol-5-yl)cyclopropanecarboxylic acid

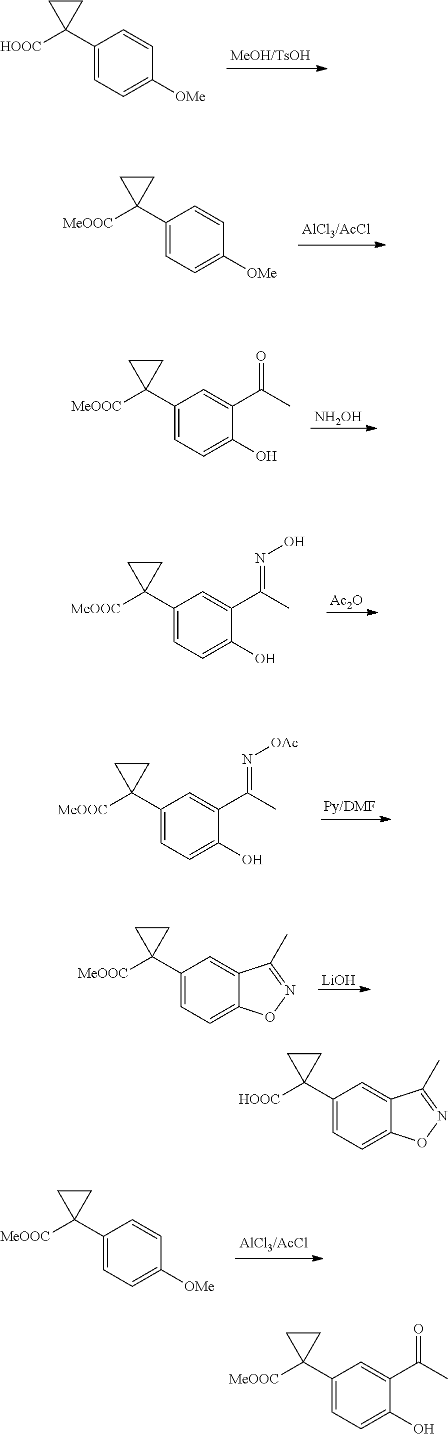

### 1-(3-Acetyl-4-hydroxy-phenyl)-cyclopropanecarboxylic methyl ester

To a stirred suspension of AlCl₃ (58 g, 440 mmol) in CS₂ (500 mL) was added acetyl chloride (7.4 g, 95 mmol) at room temperature. After stirring for 5 min, methyl 1-(4-methoxyphenyl)cyclopropanecarboxylate (15 g, 73 mmol) was added. The reaction mixture was heated at reflux for 2 h before ice water was added carefully to the mixture at room temperature. The resulting mixture was extracted with EtOAc (150 mL×3). The combined organic extracts were dried over anhydrous Na₂SO₄ and evaporated under reduced pressure to give 1-(3-acetyl-4-hydroxy-phenyl)-cyclopropanecarboxylic methyl ester (15 g, 81%), which was used in the next step without further purification. ¹H NMR (CDCl₃, 400 MHz) δ 12.28 (s, 1H), 7.67 (d, J=2.0 Hz, 1H), 7.47 (dd, J=2.0, 8.4 Hz, 1H), 6.94 (d, J=8.4 Hz, 1H), 3.64 (s, 3H), 2.64 (s, 3H), 1.65-1.62 (m, 2H), 1.18-1.16 (m, 2H).

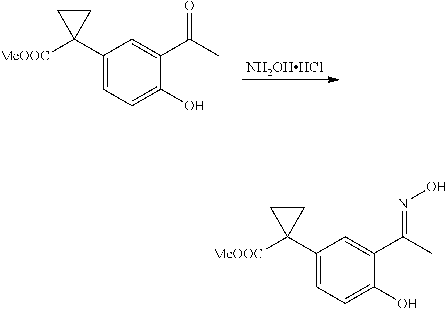

### 1-[4-Hydroxy-3-(1-hydroxyimino-ethyl)-phenyl]-cyclopropanecarboxylic methyl ester

To a stirred solution of 1-(3-acetyl-4-hydroxy-phenyl)-cyclopropanecarboxylic methyl ester (14.6 g, 58.8 mmol) in EtOH (500 mL) were added hydroxylamine hydrochloride (9.00 g, 129 mmol) and sodium acetate (11.6 g, 141 mmol) at room temperature. The resulting mixture was heated at reflux overnight. After removal of EtOH under vacuum, water (200 mL) and EtOAc (200 mL) were added. The organic layer was separated and the aqueous layer was extracted with EtOAc (100 mL×3). The combined organic layers were dried over anhydrous Na₂SO₄ and evaporated under vacuum to give 1-[4-hydroxy-3-(1-hydroxyimino-ethyl)-phenyl]-cyclopropanecarboxylic methyl ester (14.5 g, 98%), which was used in the next step without further purification. ¹H NMR (CDCl₃,400 MHz) δ 11.09 (s, 1H), 7.39 (d, J=2.0 Hz, 1H), 7.23 (d, J=2.0 Hz, 1H), 7.14 (s, 1H), 6.91 (d, J=8.4 Hz, 1H), 3.63 (s, 3H), 2.36 (s, 3H), 1.62-1.59 (m, 2H), 1.18-1.15 (m, 2H).

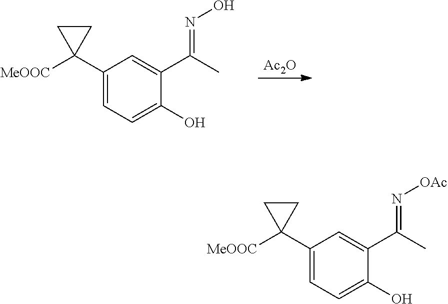 [SMILES: "Ac2O" ; COC(=O)C1(c2ccc(O)c(/C(C)=N/O)c2)CC1 ; COC(=O)C1(c2ccc(O)c(/C(C)=N/OC(C)=O)c2)CC1]

### (E)-Methyl 1-(3-(1-(acetoxyimino)ethyl)-4-hydroxyphenyl)cyclopropane carboxylate

The solution of 1-[4-hydroxy-3-(1-hydroxyimino-ethyl)-phenyl]-cyclopropanecarboxylic methyl ester (10.0 g, 40.1 mmol) in Ac₂O (250 mL) was heated at 45° C. for 4 h. The Ac₂O was removed by evaporation under vacuum before water (100 mL) and EtOAc (100 mL) were added. The organic layer was separated and the aqueous layer was extracted with EtOAc (100 mL×2). The combined organic layers were dried over anhydrous Na₂SO₄ and evaporated under vacuum to give (E)-methyl 1-(3-(1-(acetoxyimino)ethyl)-4-hydroxyphenyl)cyclopropanecarboxylate (10.5 g, 99%), which was used in the next step without further purification.

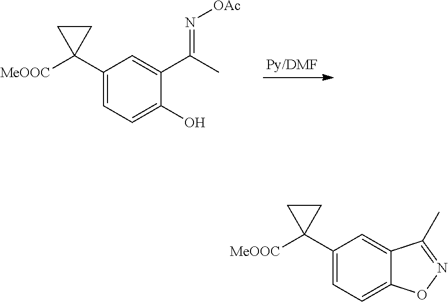 [SMILES: COC(=O)C1(c2ccc(O)c(/C(C)=N/OC(C)=O)c2)CC1 ; COC(=O)C1(c2ccc3onc(C)c3c2)CC1]

### Methyl 1-(3-methylbenzo[d]isoxazol-5-yl)cyclopropanecarboxylate

A solution of (E)-methyl 1-(3-(1-(acetoxyimino)ethyl)-4-hydroxyphenyl)cyclopropane carboxylate (10.5 g, 39.6 mmol) and pyridine (31.3 g, 396 mmol) in DMF (150 mL) was heated at 125° C. for 10 h. The cooled reaction mixture was poured into water (250 mL) and was extracted with EtOAc (100 mL×3). The combined organic layers were dried over anhydrous Na₂SO₄ and evaporated under vacuum to give the crude product, which was purified by column chromatography on silica gel (petroleum ether/ethyl acetate 50:1) to give methyl 1-(3-methylbenzo[d]isoxazol-5-yl)cyclopropanecarboxylate (7.5 g, 82%). ¹H NMR (CDCl₃ 300 MHz) δ 7.58-7.54 (m, 2H), 7.48 (dd, J=1.5, 8.1 Hz, 1H), 3.63 (s, 3H), 2.58 (s, 3H), 1.71-1.68 (m, 2H), 1.27-1.23 (m, 2H).

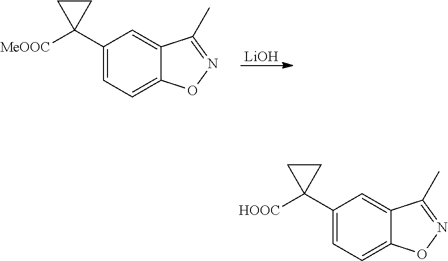

### 1-(3-Methylbenzo[d]isoxazol-5-yl)cyclopropanecarboxylic acid

To a solution of methyl 1-(3-methylbenzo[d]isoxazol-5-yl)cyclopropanecarboxylate (1.5 g, 6.5 mmol) in MeOH (20 mL) and water (2 mL) was added LiOH.H₂O (0.80 g, 19 mmol) in portions at room temperature. The reaction mixture was stirred at room temperature overnight before the MeOH was removed by evaporation under vacuum. Water and Et₂O were added and the aqueous layer was separated, acidified with HCl and extracted with EtOAc (50 mL×3). The combined organic extracts were dried over anhydrous Na₂SO₄ and evaporated under vacuum to give 1-(3-methylbenzo[d]isoxazol-5-yl)cyclopropanecarboxylic acid (455 mg, 32%). ¹H NMR (400 MHz, DMSO) δ 12.40 (br s, 1H), 7.76 (s, 1H), 7.60-7.57 (m, 2H), 2.63 (s, 3H), 1.52-1.48 (m, 2H), 1.23-1.19 (m, 2H). MS (ESI) m/z (M+H⁺) 218.1.

### Example 26

### 1-(Spiro[benzo[d][1,3]dioxole-2,1′-cyclobutane]-5-yl)cyclopropane carboxylic acid

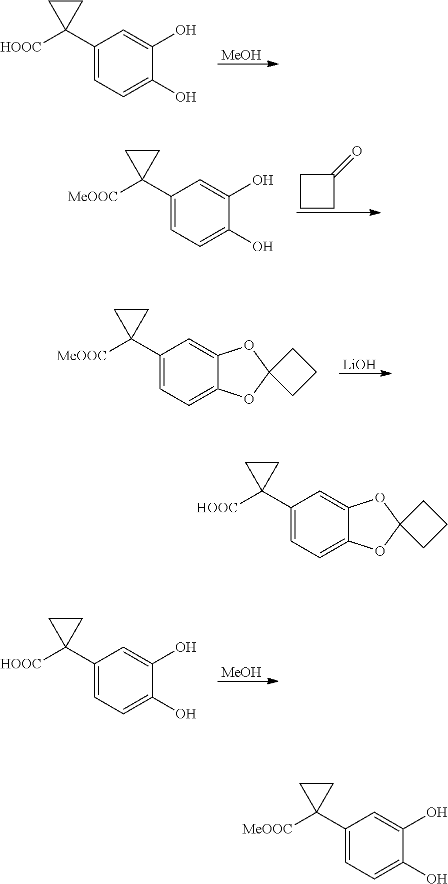

### 1-(3,4-Dihydroxy-phenyl)-cyclopropanecarboxylic methyl ester

To a solution of 1-(3,4-dihydroxyphenyl)cyclopropanecarboxylic acid (4.5 g) in MeOH (30 mL) was added TsOH (0.25 g, 1.3 mmol). The stirring was continued at 50° C. overnight before the mixture was cooled to room temperature. The mixture was concentrated under vacuum and the residue was purified by column chromatography on silica gel (petroleum ether/ethyl acetate 3:1) to give 1-(3,4-dihydroxy-phenyl)-cyclopropanecarboxylic methyl ester (2.1 g). ¹H NMR (DMSO 300 MHz) δ 8.81 (brs, 2H), 6.66 (d, J=2.1 Hz, 1H), 6.61 (d, J=8.1 Hz, 1H), 6.53 (dd, J=2.1, 8.1 Hz, 1H), 3.51 (s, 3H), 1.38-1.35 (m, 2H), 1.07-1.03 (m, 2H).

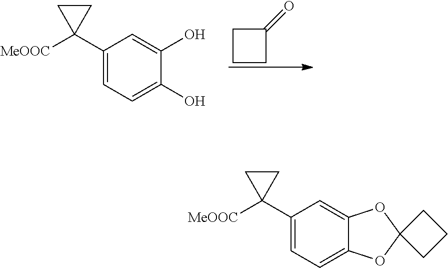

### Methyl 1-(spiro[benzo[d][1,3]dioxole-2,1′-cyclobutane]-5-yl)cyclopropane carboxylate

To a solution of 1-(3,4-dihydroxy-phenyl)-cyclopropanecarboxylic methyl ester (1.0 g, 4.8 mmol) in toluene (30 mL) was added TsOH (0.10 g, 0.50 mmol) and cyclobutanone (0.70 g, 10 mmol). The reaction mixture was heated at reflux for 2 h before being concentrated under vacuum. The residue was purified by chromatography on silica gel (petroleum ether/ethyl acetate 15:1) to give methyl 1-(spiro[benzo[d][1,3]dioxole-2,1′-cyclobutane]-5-yl)cyclopropanecarboxylate (0.6 g, 50%). ¹H NMR (CDCl₃ 300 MHz) δ 6.78-6.65 (m, 3H), 3.62 (s, 3H), 2.64-2.58 (m, 4H), 1.89-1.78 (m, 2H), 1.56-1.54 (m, 2H), 1.53-1.12 (m, 2H).

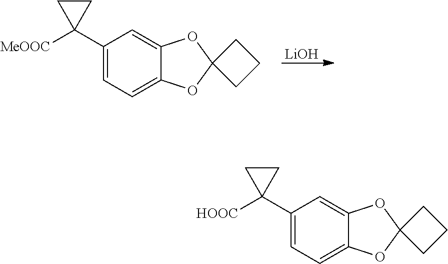

### 1-(Spiro[benzo[d][1,3]dioxole-2,1′-cyclobutane]-5-yl)cyclopropane carboxylic acid

To a mixture of methyl 1-(spiro[benzo[d][1,3]dioxole-2,1′-cyclobutane]-5-yl)cyclopropanecarboxylate (0.60 g, 2.3 mmol) in THF/H₂O (4:1, 10 mL) was added LiOH (0.30 g, 6.9 mmol). The mixture was stirred at 60° C. for 24 h. HCl (0.5N) was added slowly to the mixture at 0° C. until pH 2-3. The mixture was extracted with EtOAc (10 mL×3). The combined organic phases were washed with brine, dried over anhydrous MgSO₄, and washed with petroleum ether to give 1-(spiro[benzo[d][1,3]-dioxole-2,1′-cyclobutane]-5-yl)cyclopropane carboxylic acid (330 mg, 59%). ¹H NMR (400 MHz, CDCl₃) δ 6.78-6.65 (m, 3H), 2.65-2.58 (m, 4H), 1.86-1.78 (m, 2H), 1.63-1.60 (m, 2H), 1.26-1.19 (m, 2H).

### Example 27

### 2-(2,3-Dihydrobenzo[b][1,4]dioxin-6-yl)acetonitrile

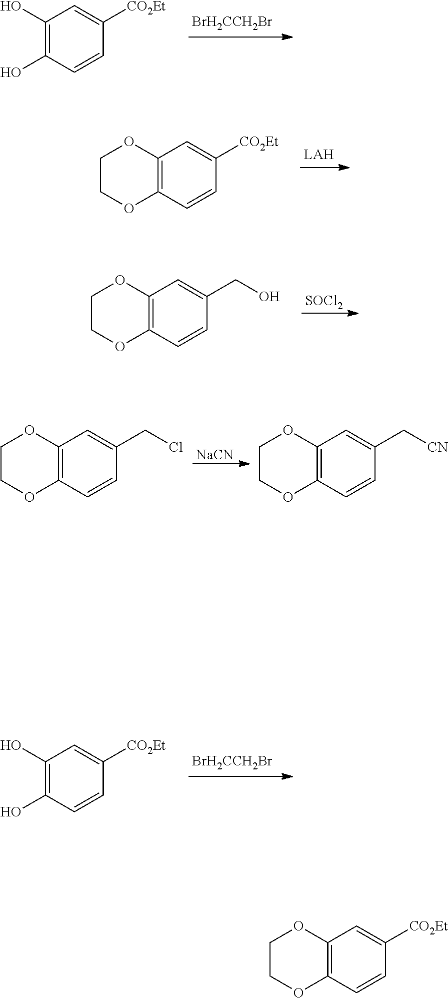

### 2,3-Dihydro-benzo[1,4]dioxine-6-carboxylic acid ethyl ester

To a suspension of Cs₂CO₃ (270 g, 1.49 mol) in DMF (1000 mL) were added 3,4-dihydroxybenzoic acid ethyl ester (54.6 g, 0.3 mol) and 1,2-dibromoethane (54.3 g, 0.29 mol) at room temperature. The resulting mixture was stirred at 80° C. overnight and then poured into ice-water. The mixture was extracted with EtOAc (200 mL×3). The combined organic layers were washed with water (200 mL×3) and brine (100 mL), dried over Na₂SO₄ and concentrated to dryness. The residue was purified by column (petroleum ether/ethyl acetate 50:1) on silica gel to obtain 2,3-dihydro-benzo[1,4]dioxine-6-carboxylic acid ethyl ester (18 g, 29%). ¹H NMR (300 MHz, CDCl₃) δ 7.53 (dd, J=1.8, 7.2 Hz, 2H), 6.84-6.87 (m, 1H), 4.22-4.34 (m, 6H), 1.35 (t, J=7.2 Hz, 3H).

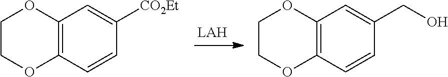 [SMILES: CCOC(=O)c1ccc2c(c1)OCCO2 ; OCc1ccc2c(c1)OCCO2]

### (2,3-Dihydro-benzo[1,4]dioxin-6-yl)-methanol

To a suspension of LiAlH₄ (2.8 g, 74 mmol) in THF (20 mL) was added dropwise a solution of 2,3-dihydro-benzo[1,4]dioxine-6-carboxylic acid ethyl ester (15 g, 72 mmol) in THF (10 mL) at 0° C. under N₂. The mixture was stirred at room temperature for 1 h and then quenched carefully with addition of water (2.8 mL) and NaOH (10%, 28 mL) with cooling. The precipitated solid was filtered off and the filtrate was evaporated to dryness to obtain (2,3-dihydro-benzo[1,4]dioxin-6-yl)-methanol (10.6 g). ¹H NMR (300 MHz, DMSO-d₆) δ 6.73-6.78 (m, 3H), 5.02 (t, J=5.7 Hz, 1H), 4.34 (d, J=6.0 Hz, 2H), 4.17-4.20 (m, 4H).

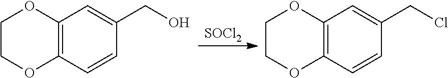

### 6-Chloromethyl-2,3-dihydro-benzo[1,4]dioxine

A mixture of (2,3-dihydro-benzo[1,4]dioxin-6-yl)methanol (10.6 g) in SOCl₂ (10 mL) was stirred at room temperature for 10 min and then poured into ice-water. The organic layer was separated and the aqueous phase was extracted with dichloromethane (50 mL×3). The combined organic layers were washed with NaHCO₃ (sat solution), water and brine, dried over Na₂SO₄ and concentrated to dryness to obtain 6-chloromethyl-2,3-dihydro-benzo[1,4]dioxine (12 g, 88% over two steps), which was used directly in next step.

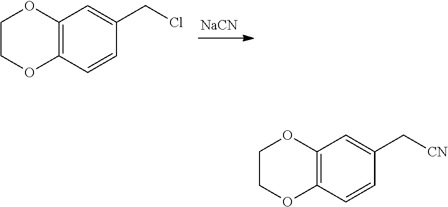

### 2-(2,3-Dihydrobenzo[b][1,4]dioxin-6-yl)acetonitrile

A mixture of 6-chloromethyl-2,3-dihydro-benzo[1,4]dioxine (12.5 g, 67.7 mmol) and NaCN (4.30 g, 87.8 mmol) in DMSO (50 mL) was stirred at rt for 1 h. The mixture was poured into water (150 mL) and then extracted with dichloromethane (50 mL×4). The combined organic layers were washed with water (50 mL×2) and brine (50 mL), dried over Na₂SO₄ and concentrated to dryness. The residue was purified by column (petroleum ether/ethyl acetate 50:1) on silica gel to obtain 2-(2,3-dihydrobenzo[b][1,4]dioxin-6-yl)acetonitrile as a yellow oil (10.2 g, 86%). ¹H-NMR (300 MHz, CDCl₃) δ 6.78-6.86 (m, 3H), 4.25 (s, 4H), 3.63 (s, 2H).

The following Table II.D-2 contains a list of carboxylic acid building blocks that were commercially available, or prepared by one of the three methods described above:

TABLE II.D-2
Carboxylic acid building blocks.

| Name | Structure |
|---|---|
| 1-benzo[1,3]dioxol-5-ylcyclopropane-1-carboxylic acid | 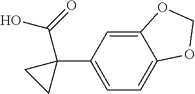 [SMILES: O=C(O)C1(c2ccc3c(c2)OCO3)CC1] |
| 1-(2,2-difluorobenzo[1,3]dioxol-5-yl)cyclopropane- 1-carboxylic acid | 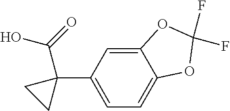 [SMILES: O=C(O)C1(c2ccc3c(c2)OC(F)(F)O3)CC1] |
| 1-(3,4-dihydroxyphenyl)cyclopropanecarboxylic acid | 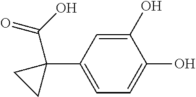 [SMILES: O=C(O)C1(c2ccc(O)c(O)c2)CC1] |
| 1-(3-methoxyphenyl)cyclopropane-1-carboxylic acid | 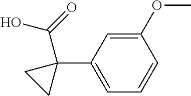 |
| 1-(2-methoxyphenyl)cyclopropane-1-carboxylic acid | 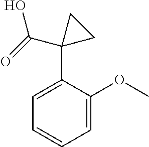 |
| 1-[4-(trifluoromethoxy)phenyl]cyclopropane-1- carboxylic acid | 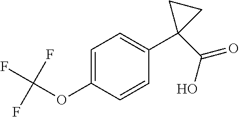 [SMILES: O=C(O)C1(c2ccc(OC(F)(F)F)cc2)CC1] |
| 1-(2,2-dimethylbenzo[d][1,3]dioxol-5- yl)cyclopropanecarboxylic acid | 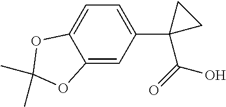 [SMILES: CC1(C)Oc2ccc(C3(C(=O)O)CC3)cc2O1] |
| tetrahydro-4-(4-methoxyphenyl)-2H-pyran-4- carboxylic acid | 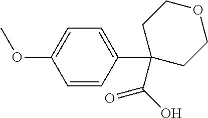 [SMILES: COc1ccc(C2(C(=O)O)CCOCC2)cc1] |
| 1-phenylcyclopropane-1-carboxylic acid | 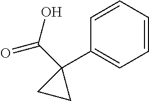 [SMILES: O=C(O)C1(c2ccccc2)CC1] |
| 1-(4-methoxyphenyl)cyclopropane-1-carboxylic acid | 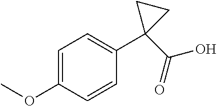 |
| 1-(4-chlorophenyl)cyclopropane-1-carboxylic acid | 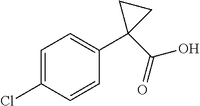 [SMILES: O=C(O)C1(c2ccc(Cl)cc2)CC1] |
| 1-(3-hydroxyphenyl)cyclopropanecarboxylic acid | 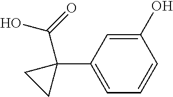 [SMILES: O=C(O)C1(c2cccc(O)c2)CC1] |
| 1-phenylcyclopentanecarboxylic acid | 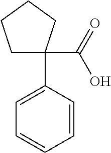 [SMILES: O=C(O)C1(c2ccccc2)CCCC1] |
| 1-(2-oxo-2,3-dihydrobenzo[d]oxazol-5- yl)cyclopropanecarboxylic acid | 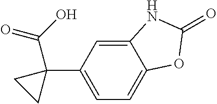 [SMILES: O=C(O)C1(c2ccc3oc(=O)[nH]c3c2)CC1] |
| 1-(benzofuran-5-yl)cyclopropanecarboxylic acid | 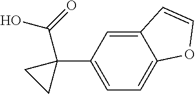 [SMILES: O=C(O)C1(c2ccc3occc3c2)CC1] |
| 1-(4-methoxyphenyl)cyclohexanecarboxylic acid | 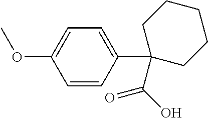 [SMILES: COc1ccc(C2(C(=O)O)CCCCC2)cc1] |
| 1-(4-chlorophenyl)cyclohexanecarboxylic acid | 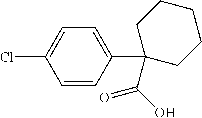 [SMILES: O=C(O)C1(c2ccc(Cl)cc2)CCCCC1] |
| 1-(2,3-dihydrobenzofuran-5- yl)cyclopropanecarboxylic acid | 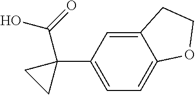 [SMILES: O=C(O)C1(c2ccc3c(c2)CCO3)CC1] |
| 1-(3,3-dimethyl-2,3-dihydrobenzofuran-5- yl)cyclopropanecarboxylic acid | 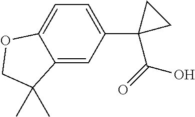 [SMILES: CC1(C)COc2ccc(C3(C(=O)O)CC3)cc21] |
| 1-(7-methoxybenzo[d][1,3]dioxol-5- yl)cyclopropanecarboxylic acid | 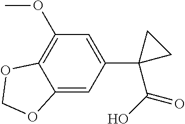 [SMILES: COc1cc(C2(C(=O)O)CC2)cc2c1OCO2] |
| 1-(3-hydroxy-4- methoxyphenyl)cyclopropanecarboxylic acid | 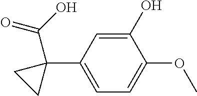 |
| 1-(4-chloro-3- hydroxyphenyl)cyclopropanecarboxylic acid | 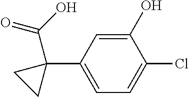 [SMILES: O=C(O)C1(c2ccc(Cl)c(O)c2)CC1] |
| 1-(3-(benzyloxy)-4- chlorophenyl)cyclopropanecarboxylic acid | 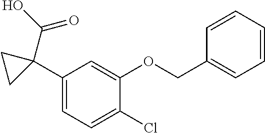 [SMILES: O=C(O)C1(c2ccc(Cl)c(OCc3ccccc3)c2)CC1] |
| 1-(4-chlorophenyl)cyclopentanecarboxylic acid | 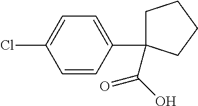 [SMILES: O=C(O)C1(c2ccc(Cl)cc2)CCCC1] |
| 1-(3-(benzyloxy)-4- methoxyphenyl)cyclopropanecarboxylic acid | 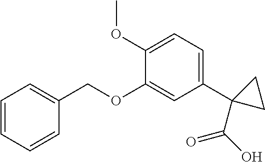 [SMILES: COc1ccc(C2(C(=O)O)CC2)cc1OCc1ccccc1] |
| 1-(3-chloro-4- methoxyphenyl)cyclopropanecarboxylic acid | 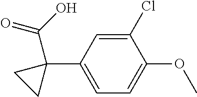 |
| 1-(3-fluoro-4- methoxyphenyl)cyclopropanecarboxylic acid | 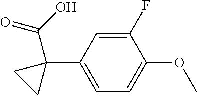 |
| 1-(4-methoxy-3- methylphenyl)cyclopropanecarboxylic acid | 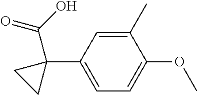 |
| 1-(4-(benzyloxy)-3- methoxyphenyl)cyclopropanecarboxylic acid | 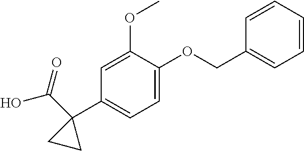 [SMILES: COc1cc(C2(C(=O)O)CC2)ccc1OCc1ccccc1] |
| 1-(4-chloro-3- methoxyphenyl)cyclopropanecarboxylic acid | 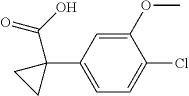 |
| 1-(3-chloro-4- hydroxyphenyl)cyclopropanecarboxylic acid | 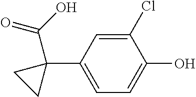 [SMILES: O=C(O)C1(c2ccc(O)c(Cl)c2)CC1] |
| 1-(3-(hydroxymethyl)-4- methoxyphenyl)cyclopropanecarboxylic acid | 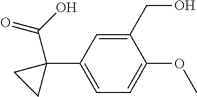 [SMILES: COc1ccc(C2(C(=O)O)CC2)cc1CO] |
| 1-(4-methoxyphenyl)cyclopentanecarboxylic acid | 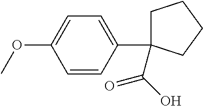 [SMILES: COc1ccc(C2(C(=O)O)CCCC2)cc1] |
| 1-phenylcyclohexanecarboxylic acid | 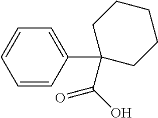 [SMILES: O=C(O)C1(c2ccccc2)CCCCC1] |
| 1-(3,4-dimethoxyphenyl)cyclopropanecarboxylic acid | 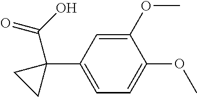 [SMILES: COc1ccc(C2(C(=O)O)CC2)cc1OC] |
| 1-(7-chlorobenzo[d][1,3]dioxol-5- yl)cyclopropanecarboxylic acid | 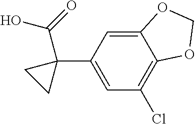 [SMILES: O=C(O)C1(c2cc(Cl)c3c(c2)OCO3)CC1] |
| 1-(benzo[d]oxazol-5-yl)cyclopropanecarboxylic acid | 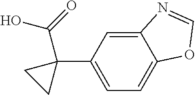 [SMILES: O=C(O)C1(c2ccc3ocnc3c2)CC1] |
| 1-(7-fluorobenzo[d][1,3]dioxol-5- yl)cyclopropanecarboxylic acid | 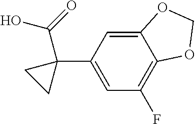 [SMILES: O=C(O)C1(c2cc(F)c3c(c2)OCO3)CC1] |
| 1-(3,4-difluorophenyl)cyclopropanecarboxylic acid | 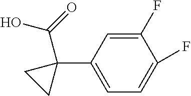 [SMILES: O=C(O)C1(c2ccc(F)c(F)c2)CC1] |
| 1-(1H-indol-5-yl)cyclopropanecarboxylic acid | 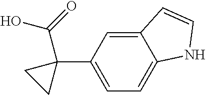 [SMILES: O=C(O)C1(c2ccc3[nH]ccc3c2)CC1] |
| 1-(1H-benzo[d]imidazol-5- yl)cyclopropanecarboxylic acid | 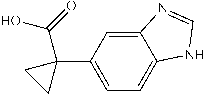 [SMILES: O=C(O)C1(c2ccc3[nH]cnc3c2)CC1] |
| 1-(2-methyl-1H-benzo[d]imidazol-5- yl)cyclopropanecarboxylic acid | 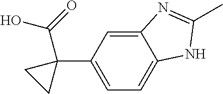 [SMILES: Cc1nc2cc(C3(C(=O)O)CC3)ccc2[nH]1] |
| 1-(1-methyl-1H-benzo[d]imidazol-5- yl)cyclopropanecarboxylic acid | 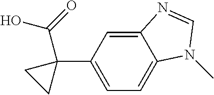 [SMILES: Cn1cnc2cc(C3(C(=O)O)CC3)ccc21] |
| 1-(3-methylbenzo[d]isoxazol-5- yl)cyclopropanecarboxylic acid | 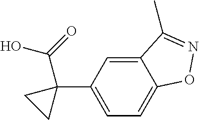 [SMILES: Cc1noc2ccc(C3(C(=O)O)CC3)cc12] |
| 1-(spiro[benzo[d][1,3]dioxole-2,1′-cyclobutane]-5- yl)cyclopropanecarboxylic acid | 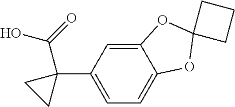 [SMILES: O=C(O)C1(c2ccc3c(c2)OC2(CCC2)O3)CC1] |
| 1-(1H-benzo[d][1,2,3]triazol-5- yl)cyclopropanecarboxylic acid | 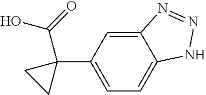 [SMILES: O=C(O)C1(c2ccc3[nH]nnc3c2)CC1] |
| 1-(1-methyl-1H-benzo[d][1,2,3]triazol-5- yl)cyclopropanecarboxylic acid | 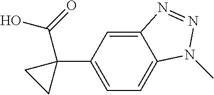 [SMILES: Cn1nnc2cc(C3(C(=O)O)CC3)ccc21] |
| 1-(1,3-dihydroisobenzofuran-5- yl)cyclopropanecarboxylic acid | 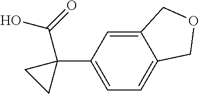 [SMILES: O=C(O)C1(c2ccc3c(c2)COC3)CC1] |
| 1-(6-fluorobenzo[d][1,3]dioxol-5- yl)cyclopropanecarboxylic acid | 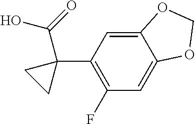 [SMILES: O=C(O)C1(c2cc3c(cc2F)OCO3)CC1] |
| 1-(2,3-dihydrobenzofuran-6- yl)cyclopropanecarboxylic acid | 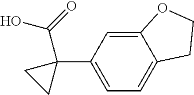 [SMILES: O=C(O)C1(c2ccc3c(c2)OCC3)CC1] |
| 1-(chroman-6-yl)cyclopropanecarboxylic acid | 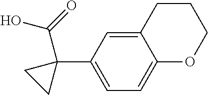 [SMILES: O=C(O)C1(c2ccc3c(c2)CCCO3)CC1] |
| 1-(4-hydroxy-4-methoxychroman-6- yl)cyclopropanecarboxylic acid | 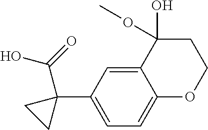 [SMILES: COC1(O)CCOc2ccc(C3(C(=O)O)CC3)cc21] |
| 1-(4-oxochroman-6-yl)cyclopropanecarboxylic acid | 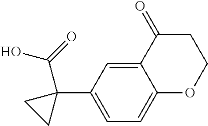 [SMILES: O=C1CCOc2ccc(C3(C(=O)O)CC3)cc21] |
| 1-(3,4-dichlorophenyl)cyclopropanecarboxylic acid | 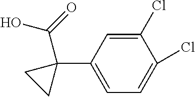 [SMILES: O=C(O)C1(c2ccc(Cl)c(Cl)c2)CC1] |
| 1-(2,3-dihydrobenzo[b][1,4]dioxin-6- yl)cyclopropanecarboxylic acid | 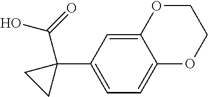 [SMILES: O=C(O)C1(c2ccc3c(c2)OCCO3)CC1] |
| 1-(benzofuran-6-yl)cyclopropanecarboxylic acid | 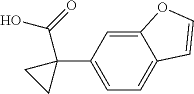 [SMILES: O=C(O)C1(c2ccc3ccoc3c2)CC1] |

### Specific Procedures

### Synthesis of Aminoindole Building Blocks

### Example 28

### 3-Methyl-1H-indol-6-amine

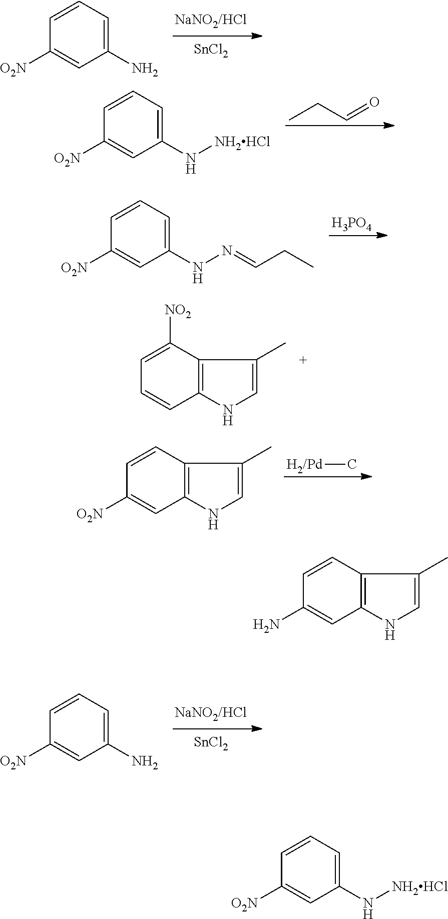

### (3-Nitro-phenyl)-hydrazine hydrochloride salt

3-Nitro-phenylamine (27.6 g, 0.2 mol) was dissolved in the mixture of H₂O (40 mL) and 37% HCl (40 mL). A solution of NaNO₂ (13.8 g, 0.2 mol) in H₂O (60 mL) was added to the mixture at 0° C., and then a solution of SnCl₂.H₂O (135.5 g, 0.6 mol) in 37% HCl (100 mL) was added at that temperature. After stirring at 0° C. for 0.5 h, the insoluble material was isolated by filtration and was washed with water to give (3-nitrophenyl)hydrazine hydrochloride (27.6 g, 73%).

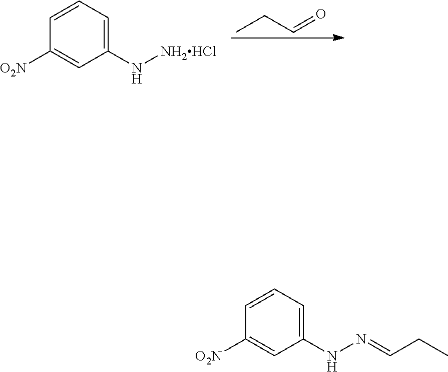

### N-(3-Nitro-phenyl)-N-propylidene-hydrazine

Sodium hydroxide solution (10%, 15 mL) was added slowly to a stirred suspension of (3-nitrophenyl)hydrazine hydrochloride (1.89 g, 10 mmol) in ethanol (20 mL) until pH 6. Acetic acid (5 mL) was added to the mixture followed by propionaldehyde (0.7 g, 12 mmol). After stirring for 3 h at room temperature, the mixture was poured into ice-water and the resulting precipitate was isolated by filtration, washed with water and dried in air to obtain (E)-1-(3-nitrophenyl)-2-propylidenehydrazine, which was used directly in the next step.

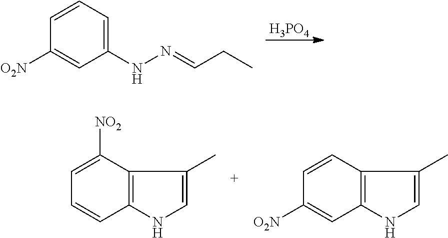

### 3-Methyl-4-nitro-1H-indole 3 and 3-methyl-6-nitro-1H-indole

A mixture of (E)-1-(3-nitrophenyl)-2-propylidenehydrazine dissolved in 85% H₃PO₄ (20 mL) and toluene (20 mL) was heated at 90-100° C. for 2 h. After cooling, toluene was removed under reduced pressure. The resultant oil was basified to pH 8 with 10% NaOH. The aqueous layer was extracted with EtOAc (100 mL three times). The combined organic layers were dried, filtered and concentrated under reduced pressure to afford the mixture of 3-methyl-4-nitro-1H-indole and 3-methyl-6-nitro-1H-indole [1.5 g in total, 86%, two steps from (3-nitrophenyl)hydrazine hydrochloride] which was used to the next step without further purification.

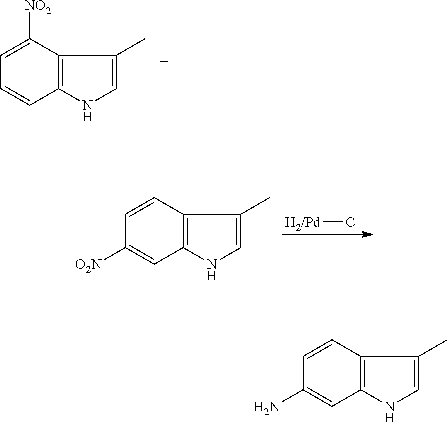

### 3-Methyl-1H-indol-6-amine

The crude mixture from previous steps (3 g, 17 mmol) and 10% Pd—C (0.5 g) in ethanol (30 mL) was stirred overnight under H₂ (1 atm) at room temperature. Pd—C was filtered off and the filtrate was concentrated under reduced pressure. The solid residue was purified by column to give 3-methyl-1H-indol-6-amine (0.6 g, 24%). ¹H NMR (CDCl₃) δ 7.59 (br s. 1H), 7.34 (d, J=8.0 Hz, 1H), 6.77 (s, 1H), 6.64 (s, 1H), 6.57 (m, 1H), 3.57 (brs, 2H), 2.28 (s, 3H); MS (ESI) m/e (M+H₊) 147.2.

### Example 29

### 3-tert-Butyl-1H-indol-5-amine

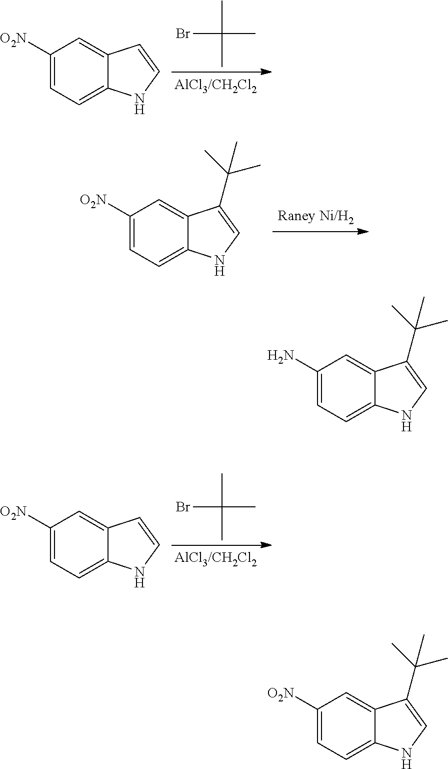

### 3-tert-Butyl-5-nitro-1H-indole

To a mixture of 5-nitro-1H-indole (6.0 g, 37 mmol) and AlCl₃ (24 g, 0.18 mol) in CH₂Cl₂ (100 mL) at 0° C. was added 2-bromo-2-methyl-propane (8.1 g, 37 mmol) dropwise. After being stirred at 15° C. overnight, the mixture was poured into ice (100 mL). The precipitated salts were removed by filtration and the aqueous layer was extracted with CH₂Cl₂ (30 mL×3). The combined organic layers were washed with water, brine, dried over Na₂SO₄ and concentrated under vacuum to obtain the crude product, which was purified by column chromatography on silica gel (petroleum ether/ethyl acetate=20:1) to give 3-tert-butyl-5-nitro-1H-indole (2.5 g, 31%). ¹H NMR (CDCl₃, 400 MHz) δ 8.49 (d, J=1.6 Hz, 1H), 8.31 (brs, 1H), 8.05 (dd, J=2.0, 8.8 Hz, 1H), 7.33 (d, J=8.8 Hz, 1H), 6.42 (d, J=1.6 Hz, 1H), 1.42 (s, 9H).

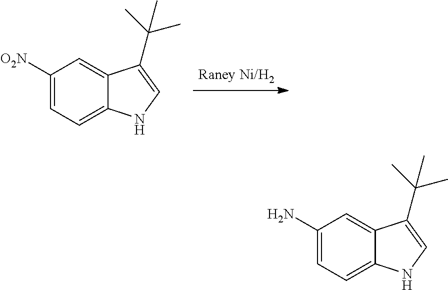 [SMILES: CC(C)(C)c1c[nH]c2ccc([N+](=O)[O-])cc12 ; CC(C)(C)c1c[nH]c2ccc(N)cc12]

### 3-tert-Butyl-1H-indol-5-amine

To a solution of 3-tert-butyl-5-nitro-1H-indole (2.5 g, 12 mmol) in MeOH (30 mL) was added Raney Nickel (0.2 g) under N₂ protection. The mixture was stirred under hydrogen atmosphere (1 atm) at 15° C. for 1 h. The catalyst was filtered off and the filtrate was concentrated to dryness under vacuum. The residue was purified by preparative HLPC to afford 3-tert-butyl-1H-indol-5-amine (0.43 g, 19%). ¹H NMR (CDCl₃, 400 MHz) δ 7.72 (br.s, 1H), 7.11 (d, J=8.4 Hz, 1H), 6.86 (d, J=2.0 Hz, 1H), 6.59 (dd, J=2.0, 8.4 Hz, 1H), 6.09 (d, J=1.6 Hz, 1H), 1.37 (s, 9H); MS (ESI) m/e (M+H⁺) 189.1.

### Example 30

### 2-tert-Butyl-6-fluoro-1H-indol-5-amine and 6-tert-butoxy-2-tert-butyl-1H-indol-5-amine

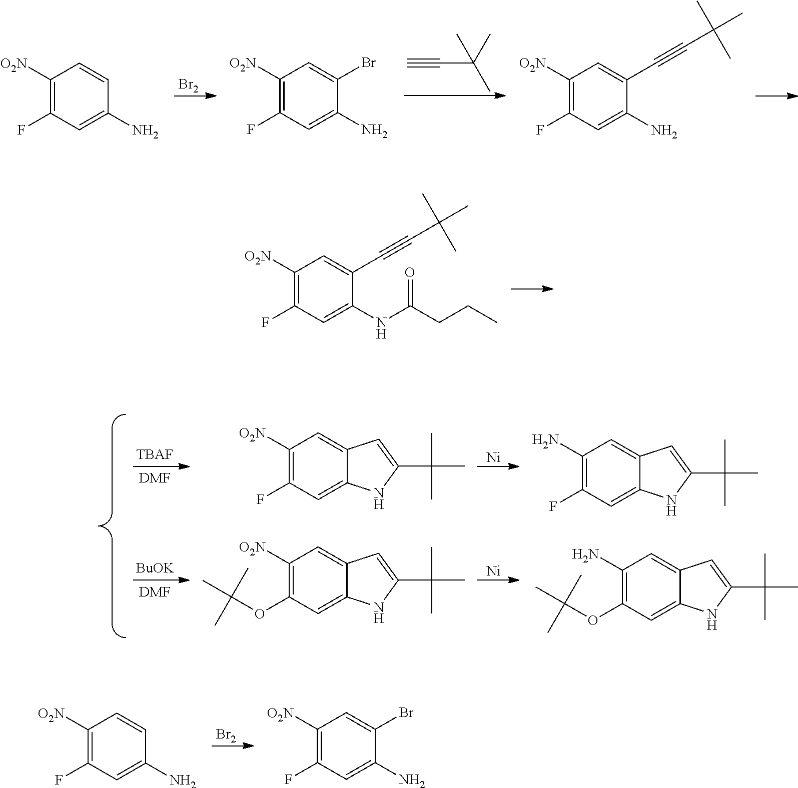

### 2-Bromo-5-fluoro-4-nitroaniline

To a mixture of 3-fluoro-4-nitroaniline (6.5 g, 42.2 mmol) in AcOH (80 mL) and chloroform (25 mL) was added dropwise Br₂ (2.15 mL, 42.2 mmol) at 0° C. After addition, the resulting mixture was stirred at room temperature for 2 h and then poured into ice water. The mixture was basified with aqueous NaOH (10%) to pH ˜8.0-9.0 under cooling and then extracted with EtOAc (50 mL×3). The combined organic layers were washed with water (80 mL×2) and brine (100 mL), dried over Na₂SO₄ and concentrated under reduced pressure to give 2-bromo-5-fluoro-4-nitroaniline (9 g, 90%). ¹H-NMR (400 MHz, DMSO-d₆) δ 8.26 (d, J=8.0, Hz, 1H), 7.07 (brs, 2H), 6.62 (d, J=9.6 Hz, 1H).

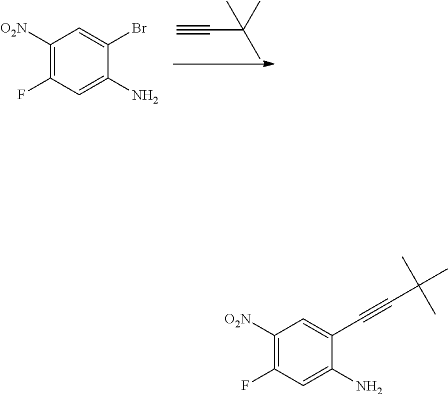

### 2-(3,3-Dimethylbut-1-ynyl)-5-fluoro-4-nitroaniline

A mixture of 2-bromo-5-fluoro-4-nitroaniline (9.0 g, 38.4 mmol), 3,3-dimethyl-but-1-yne (9.95 g, 121 mmol), CuI (0.5 g 2.6 mmol), Pd(PPh₃)2Cl₂ (3.4 g, 4.86 mmol) and Et₃N (14 mL, 6.9 mmol) in toluene (100 mL) and water (50 mL) was heated at 70° C. for 4 h. The aqueous layer was separated and the organic layer was washed with water (80 mL×2) and brine (100 mL), dried over Na₂SO₄ and concentrated under reduced pressure to dryness. The residue was recrystallized with ether to afford 2-(3,3-dimethylbut-1-ynyl)-5-fluoro-4-nitroaniline (4.2 g, 46%). ¹H-NMR (400 MHz, DMSO-d₆) δ 7.84 (d, J=8.4 Hz, 1H), 6.84 (brs, 2H), 6.54 (d, J=14.4 Hz, 1H), 1.29 (s, 9H).

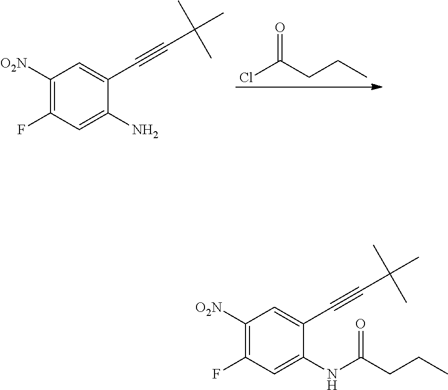

### N-(2-(3,3-Dimethylbut-1-ynyl)-5-fluoro-4-nitrophenyl)butyramide

To a solution of 2-(3,3-dimethylbut-1-ynyl)-5-fluoro-4-nitroaniline (4.2 g, 17.8 mmol) in dichloromethane (50 mL) and Et₃N (10.3 mL, 71.2 mmol) was added butyryl chloride (1.9 g, 17.8 mmol) at 0° C. The mixture was stirred at room temperature for 1 h and then poured into water. The aqueous phase was separated and the organic layer was washed with water (50 mL×2) and brine (100 mL), dried over Na₂SO₄ and concentrated under reduced pressure to dryness. The residue was washed with ether to give N-(2-(3,3-dimethylbut-1-ynyl)-5-fluoro-4-nitrophenyl)butyramide (3.5 g, 67%), which was used in the next step without further purification.

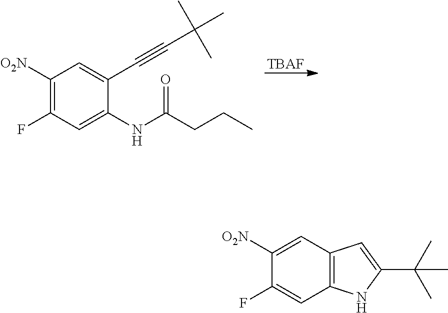

### 2-tert-Butyl-6-fluoro-5-nitro-1H-indole

A solution of N-(2-(3,3-dimethylbut-1-ynyl)-5-fluoro-4-nitrophenyl)butyramide (3.0 g, 9.8 mmol) and TBAF (4.5 g, 17.2 mmol) in DMF (25 mL) was heated at 100° C. overnight. The mixture was poured into water and then extracted with EtOAc (80 mL×3). The combined extracts were washed with water (50 mL) and brine (50 mL), dried over Na₂SO₄ and concentrated under reduced pressure to dryness. The residue was purified by column chromatography on silica gel (petroleum ether/ethyl acetate 20:1) to give compound 2-tert-butyl-6-fluoro-5-nitro-1H-indole (1.5 g, 65%). ¹H-NMR (400 MHz, CDCl₃) δ 8.30 (d, J=7.2 Hz, 1H), 7.12 (d, J=11.6 Hz, 1H), 6.35 (d, J=1.2 Hz, 1H), 1.40 (s, 9H).

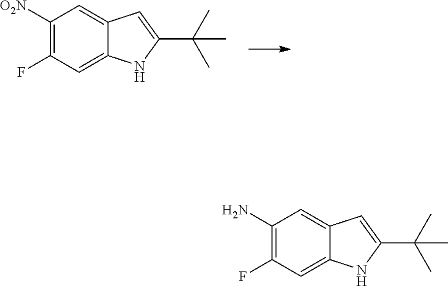 [SMILES: CC(C)(C)c1cc2cc([N+](=O)[O-])c(F)cc2[nH]1 ; CC(C)(C)c1cc2cc(N)c(F)cc2[nH]1]

### 2-tert-Butyl-6-fluoro-1H-indol-5-amine

A suspension of 2-tert-butyl-6-fluoro-5-nitro-1H-indole (1.5 g, 6.36 mmol) and Ni (0.5 g) in MeOH (20 mL) was stirred under H₂ atmosphere (1 atm) at the room temperature for 3 h. The catalyst was filtered off and the filtrate was concentrated under reduced pressure to dryness. The residue was recrystallized in ether to give 2-tert-butyl-6-fluoro-1H-indol-5-amine (520 mg, 38%). ¹H-NMR (300 MHz, DMSO-d₆) δ 10.46 (brs, 1H), 6.90 (d, J=8.7 Hz, 1H), 6.75 (d, J=9.0 Hz, 1H), 5.86 (s, 1H), 4.37 (brs, 2H), 1.29 (s, 9H); MS (ESI) m/e 206.6.

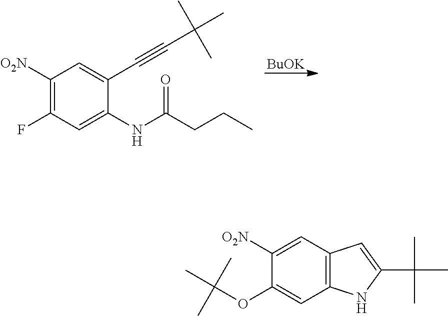 [SMILES: "BuOK" ; CCCC(=O)Nc1cc(F)c([N+](=O)[O-])cc1C#CC(C)(C)C ; CC(C)(C)Oc1cc2[nH]c(C(C)(C)C)cc2cc1[N+](=O)[O-]]

### 6-tert-Butoxy-2-tert-butyl-5-nitro-1H-indole

A solution of N-(2-(3,3-dimethylbut-1-ynyl)-5-fluoro-4-nitrophenyl)butyramide (500 mg, 1.63 mmol) and t-BuOK (0.37 g, 3.26 mmol) in DMF (10 mL) was heated at 70° C. for 2 h. The mixture was poured into water and then extracted with EtOAc (50 mL×3). The combined extracts were washed with water (50 mL) and brine (50 mL), dried over Na₂SO₄ and concentrated under reduced pressure to give 6-tert-butoxy-2-tert-butyl-5-nitro-1H-indole (100 mg, 21%). ¹H-NMR (300 MHz, DMSO-d₆) δ 11.35 (brs, 1H), 7.99 (s, 1H), 7.08 (s, 1H), 6.25 (s, 1H), 1.34 (s, 9H), 1.30 (s, 9H).

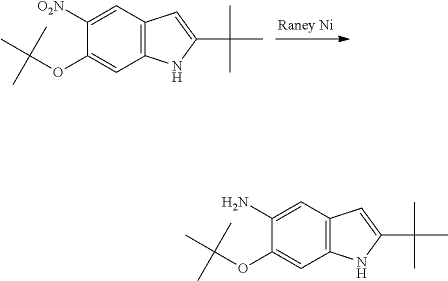 [SMILES: CC(C)(C)Oc1cc2[nH]c(C(C)(C)C)cc2cc1[N+](=O)[O-] ; CC(C)(C)Oc1cc2[nH]c(C(C)(C)C)cc2cc1N]

### 6-tert-Butoxy-2-tert-butyl-1H-indol-5-amine

A suspension of 6-tert-butoxy-2-tert-butyl-5-nitro-1H-indole (100 mg, 0.36 mmol) and Raney Ni (0.5 g) in MeOH (15 mL) was stirred under H₂ atmosphere (1 atm) at the room temperature for 2.5 h. The catalyst was filtered off and the filtrate was concentrated under reduced pressure to dryness. The residue was recrystallized in ether to give 6-tert-butoxy-2-tert-butyl-1H-indol-5-amine (30 mg, 32%). ¹H-NMR (300 MHz, MeOD) 6.98 (s, 1H), 6.90 (s, 1H), 5.94 (d, J=0.6 Hz, 1H), 1.42 (s, 9H), 1.36 (s, 9H); MS (ESI) m/e 205.0.

### Example 31

### 1-tert-Butyl-1H-indol-5-amine

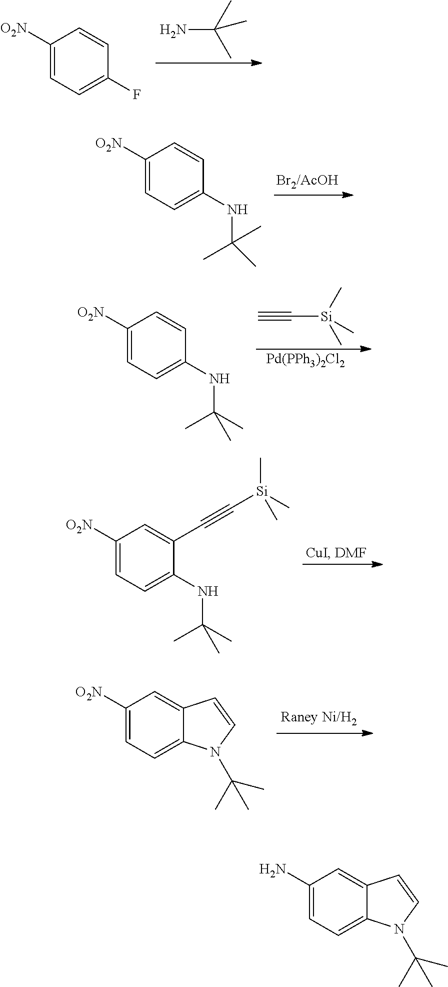

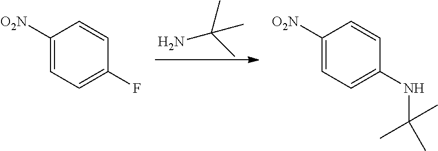

### N-tert-Butyl-4-nitroaniline

A solution of 1-fluoro-4-nitro-benzene (1 g, 7.1 mmol) and tert-butylamine (1.5 g, 21 mmol) in DMSO (5 mL) was stirred at 75° C. overnight. The mixture was poured into water (10 mL) and extracted with EtOAc (7 mL×3). The combined organic layers were washed with water, brine, dried over Na₂SO₄ and concentrated under vacuum to dryness. The residue was purified by column chromatography on silica gel (petroleum ether/ethyl acetate 30:1) to afford N-tert-butyl-4-nitroaniline (1 g, 73%). ¹H NMR (CDCl₃, 400 MHz) δ 8.03-8.00 (m, 2H), 6.61-6.57 (m, 2H), 4.67 (brs, 1H), 1.42 (s, 9H).

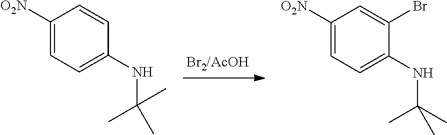

### (2-Bromo-4-nitro-phenyl)-tert-butyl-amine

To a solution of N-tert-butyl-4-nitroaniline (1 g, 5.1 mmol) in AcOH (5 mL) was added Br₂ (0.86 g, 54 mmol) dropwise at 15° C. After addition, the mixture was stirred at 30° C. for 30 min and then filtered. The filter cake was basified to pH 8-9 with aqueous NaHCO₃. The aqueous layer was extracted with EtOAc (10 mL×3). The combined organic layers were washed with water, brine, dried over Na₂SO₄ and concentrated under vacuum to give (2-bromo-4-nitro-phenyl)-tert-butyl-amine (0.6 g, 43%). ¹H-NMR (CDCl₃, 400 MHz) δ 8.37 (dd, J=2.4 Hz, 1H), 8.07 (dd, J=2.4, 9.2 Hz, 1H), 6.86 (d, J=9.2 Hz, 1H), 5.19 (brs, 1H), 1.48 (s, 9H).

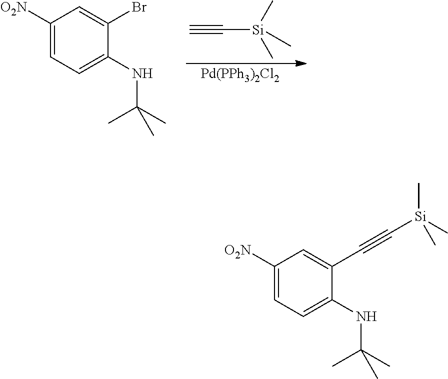

### tert-Butyl-(4-nitro-2-trimethylsilanylethynyl-phenyl)-amine

To a solution of (2-bromo-4-nitro-phenyl)-tert-butyl-amine (0.6 g, 2.2 mmol) in Et₃N (10 mL) was added Pd(PPh₃)2Cl₂ (70 mg, 0.1 mmol), CuI (20.9 mg, 0.1 mmol) and ethynyl-trimethyl-silane (0.32 g, 3.3 mmol) successively under N₂ protection. The reaction mixture was heated at 70° C. overnight. The solvent was removed under vacuum and the residue was washed with EtOAc (10 mL×3). The combined organic layers were washed with water, brine, dried over Na₂SO₄ and concentrated under vacuum to dryness. The residue was purified by column chromatography on silica gel (petroleum ether/ethyl acetate 20:1) to afford tert-butyl-(4-nitro-2-trimethylsilanylethynyl-phenyl)-amine (100 mg, 16%). ¹H-NMR (CDCl₃, 400 MHz) δ 8.20 (d, J=2.4, Hz, 1H), 8.04 (dd, J=2.4, 9.2 Hz, 1H), 6.79 (d, J=9.6 Hz, 1H), 5.62 (brs, 1H), 1.41 (s, 9H), 0.28 (s, 9H).

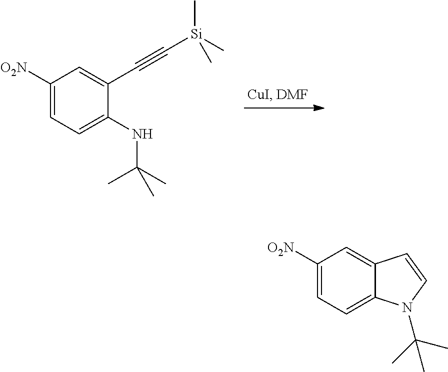 [SMILES: CC(C)(C)Nc1ccc([N+](=O)[O-])cc1C#C[Si](C)(C)C ; CC(C)(C)n1ccc2cc([N+](=O)[O-])ccc21]

### 1-tert-Butyl-5-nitro-1H-indole

To a solution of tert-butyl-(4-nitro-2-trimethylsilanylethynyl-phenyl)-amine (10 mg, 0.035 mmol) in DMF (2 mL), was added CuI (13 mg, 0.07 mmol) under N₂ protection. The reaction mixture was stirred at 100° C. overnight. At this time, EtOAc (4 mL) was added to the mixture. The mixture was filtered and the filtrate was washed with water, brine, dried over Na₂SO₄ and concentrated under vacuum to obtain 1-tert-butyl-5-nitro-1H-indole (7 mg, 93%). ¹H-NMR (CDCl₃, 300 MHz) δ 8.57 (d, J=2.1 Hz, 1H), 8.06 (dd, J=2.4, 9.3 Hz, 1H), 7.65 (d, J=9.3 Hz, 1H), 7.43 (d, J=3.3 Hz, 1H), 6.63 (d, J=3.3 Hz, 1H), 1.76 (s, 9H).

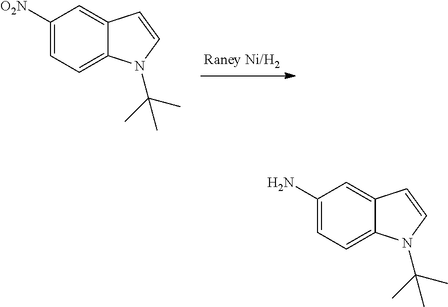 [SMILES: CC(C)(C)n1ccc2cc([N+](=O)[O-])ccc21 ; CC(C)(C)n1ccc2cc(N)ccc21]

### 1-tert-Butyl-1H-indol-5-amine

To a solution of 1-tert-butyl-5-nitro-1H-indole (6.5 g, 0.030 mol) in MeOH (100 mL) was added Raney Nickel (0.65 g, 10%) under N₂ protection. The mixture was stirred under hydrogen atmosphere (1 atm) at 30° C. for 1 h. The catalyst was filtered off and the filtrate was concentrated under vacuum to dryness. The residue was purified by column chromatography on silica gel (PE/EtOAc 1:2) to give 1-tert-butyl-1H-indol-5-amine (2.5 g, 45%). ¹H-NMR (CDCl₃, 400 MHz) δ 7.44 (d, J=8.8 Hz, 1H), 7.19 (dd, J=3.2 Hz, 1H), 6.96 (d, J=2.0 Hz, 1H), 6.66 (d, J=2.0, 8.8 Hz, 1H), 6.26 (d, J=3.2 Hz, 1H), 1.67 (s, 9H). MS (ESI) m/e (M+H⁺) 189.2.

### Example 32

### 2-tert-Butyl-1-methyl-1H-indol-5-amine

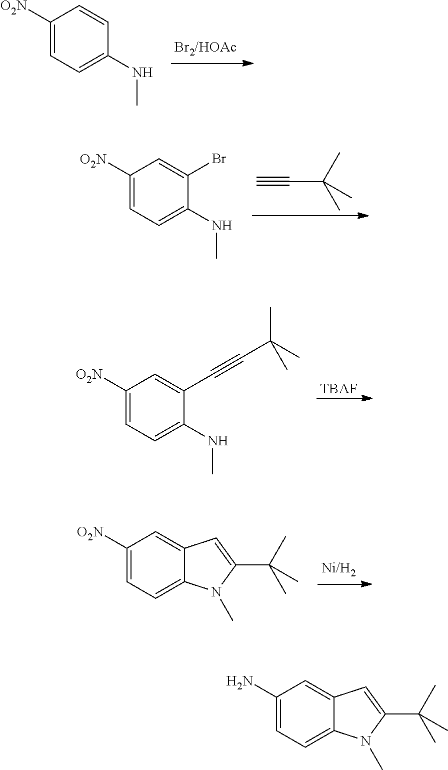

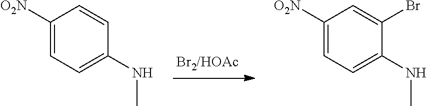

### (2-Bromo-4-nitro-phenyl)-methyl-amine

To a solution of methyl-(4-nitro-phenyl)-amine (15.2 g, 0.1 mol) in AcOH (150 mL) and CHCl₃ (50 mL) was added Br₂ (16.0 g, 0.1 mol) dropwise at 5° C. The mixture was stirred at 10° C. for 1 h and then basified with sat. aq. NaHCO₃. The resulting mixture was extracted with EtOAc (100 mL×3), and the combined organics were dried over anhydrous Na₂SO₄ and evaporated under vacuum to give (2-bromo-4-nitro-phenyl)-methyl-amine (2-bromo-4-nitro-phenyl)-methyl-amine (23.0 g, 99%), which was used in the next step without further purification. ¹H NMR (300 MHz, CDCl₃) δ 8.37 (d, J=2.4 Hz, 1H), 8.13 (dd, J=2.4, 9.0 Hz, 1H), 6.58 (d, J=9.0 Hz, 1H), 5.17 (brs, 1H), 3.01 (d, J=5.4 Hz, 3H).

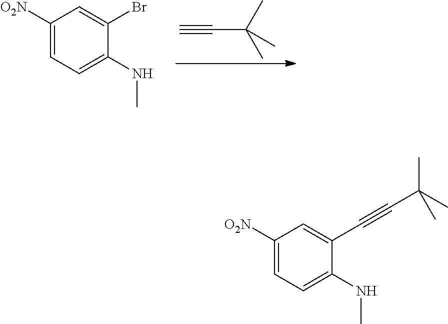

### [2-(3,3-Dimethyl-but-1-ynyl)-4-nitro-phenyl]methyl-amine

To a solution of (2-bromo-4-nitro-phenyl)-methyl-amine (22.5 g, 97.4 mmol) in toluene (200 mL) and water (100 mL) were added Et₃N (19.7 g, 195 mmol), Pd(PPh₃)₂Cl₂ (6.8 g, 9.7 mmol), CuI (0.7 g, 3.9 mmol) and 3,3-dimethyl-but-1-yne (16.0 g, 195 mmol) successively under N₂ protection. The mixture was heated at 70° C. for 3 hours and then cooled down to room temperature. The resulting mixture was extracted with EtOAc (100 mL×3). The combined organic extracts were dried over anhydrous Na₂SO₄ and evaporated under vacuum to give [2-(3,3-dimethyl-but-1-ynyl)-4-nitro-phenyl]methyl-amine (20.1 g, 94%), which was used in the next step without further purification. ¹H NMR (400 MHz, CDCl₃) δ 8.15 (d, J=2.4 Hz, 1H), 8.08 (dd, J=2.8, 9.2 Hz, 1H), 6.50 (d, J=9.2 Hz, 1H), 5.30 (brs, 1H), 3.00 (s, 3H), 1.35 (s, 9H).

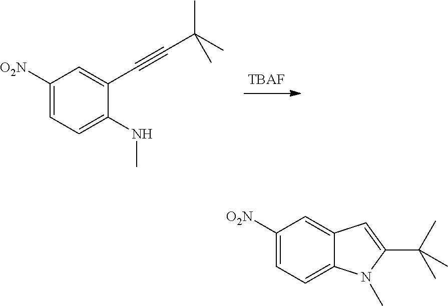

### 2-tert-Butyl-1-methyl-5-nitro-1H-indole

A solution of [2-(3,3-dimethyl-but-1-ynyl)-4-nitro-phenyl]methyl-amine (5.0 g, 22.9 mmol) and TBAF (23.9 g, 91.6 mmol) in THF (50 mL) was heated at reflux overnight. The solvent was removed by evaporation under vacuum and the residue was dissolved in brine (100 mL) and EtOAc (100 mL). The organic phase was separated, dried over Na₂SO₄ and evaporated under vacuum to give 2-tert-butyl-1-methyl-5-nitro-1H-indole (5.0 g, 99%), which was used in the next step without further purification. ¹H NMR (CDCl₃, 400 MHz) δ 8.47 (d, J=2.4 Hz, 1H), 8.07 (dd, J=2.4, 9.2 Hz, 1H), 7.26-7.28 (m, 1H), 6.47 (s, 1H), 3.94 (s, 3H), 1.50 (s, 9H).

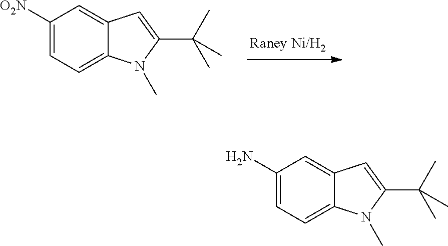 [SMILES: Cn1c(C(C)(C)C)cc2cc([N+](=O)[O-])ccc21 ; Cn1c(C(C)(C)C)cc2cc(N)ccc21]

### 2-tert-Butyl-1-methyl-1H-indol-5-amine

To a solution of 2-tert-butyl-1-methyl-5-nitro-1H-indole (3.00 g, 13.7 mmol) in MeOH (30 mL) was added Raney Ni (0.3 g) under nitrogen atmosphere. The mixture was stirred under hydrogen atmosphere (1 atm) at room temperature overnight. The mixture was filtered through a Celite pad and the filtrate was evaporated under vacuum. The crude residue was purified by column chromatography on silica gel (P.E/EtOAc 20:1) to give 2-tert-butyl-1-methyl-1H-indol-5-amine (1.7 g, 66%). ¹H NMR (300 MHz, CDCl₃) δ 7.09 (d, J=8.4 Hz, 1H), 6.89-6.9 (m, 1H), 6.66 (dd, J=2.4, 8.7 Hz, 1H), 6.14 (d, J=0.6 Hz, 1H), 3.83 (s, 3H), 3.40 (brs, 2H), 1.45 (s, 9H); MS (ESI) m/e (M+H⁺) 203.1.

### Example 33

### 2-Cyclopropyl-1H-indol-5-amine

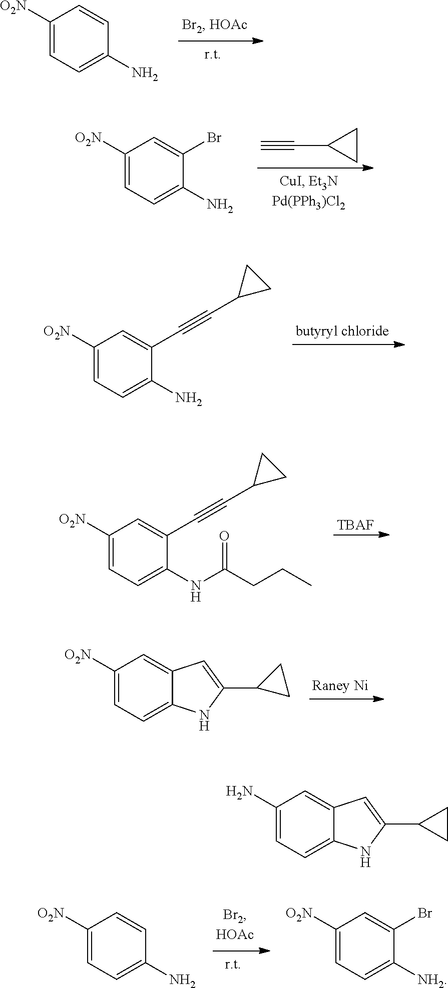

### 2-Bromo-4-nitroaniline

To a solution of 4-nitro-aniline (25 g, 0.18 mol) in HOAc (150 mL) was added liquid Br₂ (30 g, 0.19 mol) dropwise at room temperature. The mixture was stirred for 2 hours. The solid was collected by filtration and poured into water (100 mL), which was basified with sat. aq. NaHCO₃ to pH 7 and extracted with EtOAc (300 mL×3). The combined organic layers were dried over anhydrous Na₂SO₄ and evaporated under reduced pressure to give 2-bromo-4-nitroaniline (30 g, 80%), which was directly used in the next step.

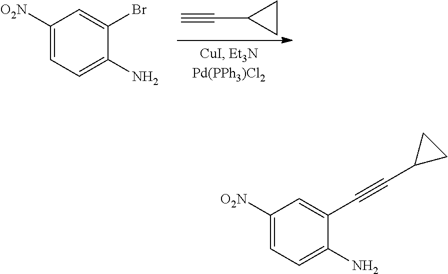

### 2-(Cyclopropylethynyl)-4-nitroaniline

To a deoxygenated solution of 2-bromo-4-nitroaniline (2.17 g, 0.01 mmol), ethynyl-cyclopropane (1 g, 15 mmol) and CuI (10 mg, 0.05 mmol) in triethylamine (20 mL) was added Pd(PPh₃)₂Cl₂ (210 mg, 0.3 mmol) under N₂. The mixture was heated at 70° C. and stirred for 24 hours. The solid was filtered off and washed with EtOAc (50 mL×3). The filtrate was evaporated under reduced pressure, and the residue was purified by column chromatography on silica gel (petroleum ether/ethyl acetate=10/1) to give 2-(cyclopropylethynyl)-4-nitroaniline (470 mg, 23%). ¹H NMR (300 MHz, CDCl₃) δ 8.14 (d, J=2.7 Hz, 1H), 7.97 (dd, J=2.7, 9.0 Hz, 1H), 6.63 (d, J=9.0 Hz, 1H), 4.81 (brs, 2H), 1.55-1.46 (m, 1H), 0.98-0.90 (m, 2H), 0.89-0.84 (m, 2H).

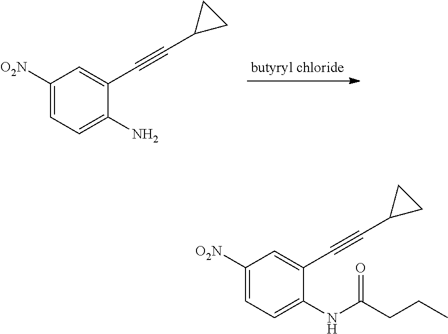 [SMILES: Nc1ccc([N+](=O)[O-])cc1C#CC1CC1 ; CCCC(=O)Nc1ccc([N+](=O)[O-])cc1C#CC1CC1]

### N-(2-(Cyclopropylethynyl)phenyl)-4-nitrobutyramide

To a solution of 2-(cyclopropylethynyl)-4-nitroaniline (3.2 g, 15.8 mmol) and pyridine (2.47 g, 31.7 mmol) in CH₂Cl₂ (60 mL) was added butyryl chloride (2.54 g, 23.8 mmol) at 0° C. The mixture was warmed to room temperature and stirred for 3 hours. The resulting mixture was poured into ice-water. The organic layer was separated. The aqueous phase was extracted with CH₂Cl₂ (30 mL×3). The combined organic layers were dried over anhydrous Na₂SO₄ and evaporated under reduced pressure to give the crude product, which was purified by column chromatography on silica gel (petroleum ether/ethyl acetate=10/1) to give N-(2-(cyclopropylethynyl)phenyl)-4-nitrobutyramide (3.3 g, 76%). ¹H NMR (400 MHz, CDCl₃) δ 8.61 (d, J=9.2 Hz, 1H), 8.22 (d, J=2.8 Hz, 1H), 8.18 (brs, 1H), 8.13 (dd, J=2.4, 9.2 Hz, 1H), 2.46 (t, J=7.2 Hz, 2H), 1.83-1.76 (m, 2H), 1.59-1.53 (m, 1H), 1.06 (t, J=7.2 Hz, 3H), 1.03-1.01 (m, 2H), 0.91-0.87 (m, 2H).

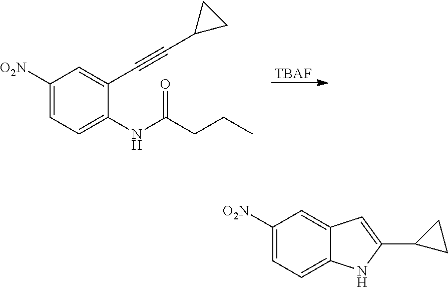

### 2-Cyclopropyl-5-nitro-1H-indole

A mixture of N-(2-(cyclopropylethynyl)phenyl)-4-nitobutyramide (3.3 g, 0.01 mol) and TBAF (9.5 g, 0.04 mol) in THF (100 mL) was heated at reflux for 24 hours. The mixture was cooled to the room temperature and poured into ice water. The mixture was extracted with CH₂Cl₂ (50 mL×3). The combined organic layers were dried over anhydrous Na₂SO₄ and evaporated under reduced pressure. The residue was purified by column chromatography on silica gel (petroleum ether/ethyl acetate=10/1) to give 2-cyclopropyl-5-nitro-1H-indole (1.3 g, 64%). ¹H NMR (400 MHz, CDCl₃) δ 8.44 (d, J=2.0 Hz, 1H), 8.40 (brs, 1H), 8.03 (dd, J=2.0, 8.8 Hz, 1H), 7.30 (d, J=8.8 Hz, 1H), 6.29 (d, J=0.8 Hz, 1H), 2.02-1.96 (m, 1H) 1.07-1.02 (m, 2H), 0.85-0.81 (m, 2H).

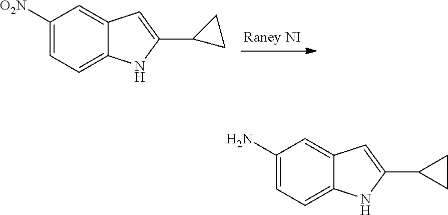 [SMILES: O=[N+]([O-])c1ccc2[nH]c(C3CC3)cc2c1 ; Nc1ccc2[nH]c(C3CC3)cc2c1]

### 2-Cyclopropyl-1H-indol-5-amine

To a solution of 2-cyclopropyl-5-nitro-1H-indole (1.3 g, 6.4 mmol) in MeOH (30 mL) was added Raney Nickel (0.3 g) under nitrogen atmosphere. The mixture was stirred under hydrogen atmosphere (1 atm) at room temperature overnight. The catalyst was filtered through a Celite pad and the filtrate was evaporated under vacuum to give the crude product, which was purified by column chromatography on silica gel (petroleum ether/ethyl acetate=5/1) to give 2-cyclopropyl-1H-indol-5-amine (510 mg, 56%). ¹H NMR (400 MHz, CDCl₃) δ 6.89 (d, J=8.4 Hz, 1H), 6.50 (d, J=1.6 Hz, 1H), 6.33 (dd, J=2.0, 8.4 Hz, 1H), 5.76 (s, 1H), 4.33 (brs, 2H), 1.91-1.87 (m, 1H), 0.90-0.85 (m, 2H), 0.70-0.66 (m, 2H); MS (ESI) m/e (M+H⁺) 173.2.

### Example 34

### 3-tert-Butyl-1H-indol-5-amine

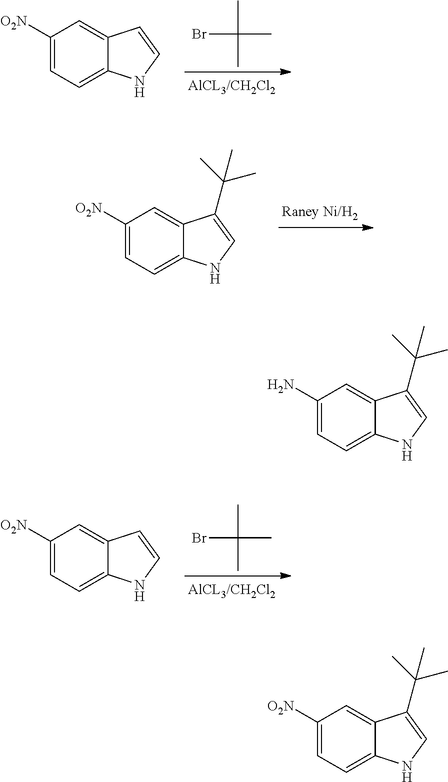

### 3-tert-Butyl-5-nitro-1H-indole

To a mixture of 5-nitro-1H-indole (6 g, 36.8 mmol) and AlCl₃ (24 g, 0.18 mol) in CH₂Cl₂ (100 mL) was added 2-bromo-2-methyl-propane (8.1 g, 36.8 mmol) dropwise at 0° C. After being stirred at 15° C. overnight, the reaction mixture was poured into ice (100 mL). The precipitated salts were removed by filtration and the aqueous layer was extracted with CH₂Cl₂ (30 mL×3). The combined organic layers were washed with water, brine, dried over Na₂SO₄ and concentrated under vacuum to obtain the crude product, which was purified by column chromatography on silica gel (petroleum ether/ethyl acetate 20:1) to give 3-tert-butyl-5-nitro-1H-indole (2.5 g, 31%). ¹H NMR (CDCl₃, 400 MHz) δ 8.49 (d, J=1.6 Hz, 1H), 8.31 (brs, 1H), 8.05 (dd, J=2.0, 8.8 Hz, 1H), 7.33 (d, J=8.8 Hz, 1H), 6.42 (d, J=1.6 Hz, 1H), 1.42 (s, 9H).

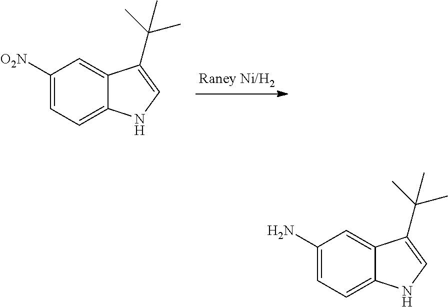 [SMILES: CC(C)(C)c1c[nH]c2ccc([N+](=O)[O-])cc12 ; CC(C)(C)c1c[nH]c2ccc(N)cc12]

### 3-tert-Butyl-1H-indol-5-amine

To a solution of 3-tert-butyl-5-nitro-1H-indole (2.5 g, 11.6 mmol) in MeOH (30 mL) was added Raney Nickel (0.2 g) under N₂ protection. The mixture was stirred under hydrogen atmosphere (1 atm) at 15° C. for 1 hr. The catalyst was filtered off and the filtrate was concentrated under vacuum to dryness. The residue was purified by preparative HLPC to afford 3-tert-butyl-1H-indol-5-amine (0.43 g, 19%). ¹H NMR (CDCl₃, 400 MHz) δ 7.72 (brs, 1H), 7.11 (d, J=8.4 Hz, 1H), 6.86 (d, J=2.0 Hz, 1H), 6.59 (dd, J=2.0, 8.4 Hz, 1H), 6.09 (d, J=1.6 Hz, 1H), 1.37 (s, 9H); MS (ESI) m/e (M+H⁺) 189.1.

### Example 35

### 2-Phenyl-1H-indol-5-amine

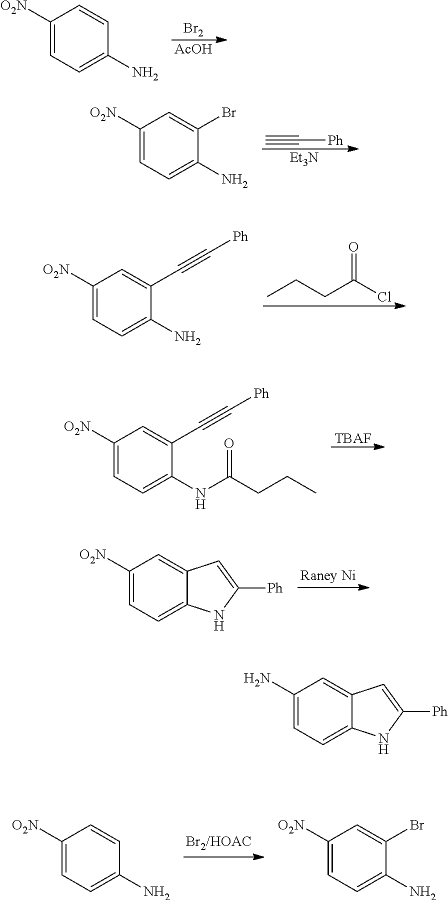

### 2-Bromo-4-nitroaniline

To a solution of 4-nitroaniline (50 g, 0.36 mol) in AcOH (500 mL) was added liquid Br₂ (60 g, 0.38 mol) dropwise at 5° C. The mixture was stirred for 30 min at that temperature. The insoluble solid was collected by filtration and poured into EtOAc (200 mL). The mixture was basified with saturated aqueous NaHCO₃ to pH 7. The organic layer was separated. The aqueous phase was extracted with EtOAc (300 mL×3). The combined organic layers were dried and evaporated under reduced pressure to give 2-bromo-4-nitroaniline (56 g, 72%), which was directly used in the next step.

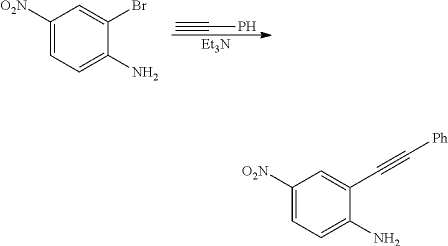

### 4-Nitro-2-(phenylethynyl)aniline

To a deoxygenated solution of 2-bromo-4-nitroaniline (2.17 g, 0.01 mmol), ethynyl-benzene (1.53 g, 0.015 mol) and CuI (10 mg, 0.05 mmol) in triethylamine (20 mL) was added Pd(PPh₃)₂Cl₂ (210 mg, 0.2 mmol) under N₂. The mixture was heated at 70° C. and stirred for 24 hours. The solid was filtered off and washed with EtOAc (50 mL×3). The filtrate was evaporated under reduced pressure and the residue was purified by column chromatography on silica gel (petroleum ether/ethyl acetate=10/1) to give 4-nitro-2-(phenylethynyl)aniline (340 mg, 14%). ¹H NMR (300 MHz, CDCl₃) δ 8.37-8.29 (m, 1H), 8.08-8.00 (m, 1H), 7.56-7.51 (m, 2H), 7.41-7.37 (m, 3H), 6.72 (m, 1H), 4.95 (brs, 2H).

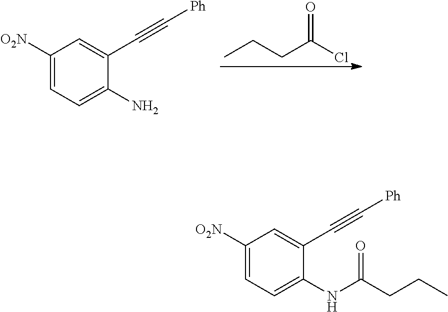

### N-(2-(Phenylethynyl)phenyl)-4-nitrobutyramide

To a solution of 4-nitro-2-(phenylethynyl)aniline (17 g, 0.07 mmol) and pyridine (11.1 g, 0.14 mol) in CH₂Cl₂ (100 mL) was added butyryl chloride (11.5 g, 0.1 mol) at 0° C. The mixture was warmed to room temperature and stirred for 3 hours. The resulting mixture was poured into ice-water. The organic layer was separated. The aqueous phase was extracted with CH₂Cl₂ (30 mL×3). The combined organic layers were dried over anhydrous Na₂SO₄ and evaporated under reduced pressure. The residue was purified by column chromatography on silica gel (petroleum ether/ethyl acetate=10/1) to give N-(2-(phenylethynyl)phenyl)-4-nitrobutyramide (12 g, 55%). ¹H NMR (400 MHz, CDCl₃) δ 8.69 (d, J=9.2 Hz, 1H), 8.39 (d, J=2.8 Hz, 1H), 8.25-8.20 (m, 2H), 7.58-7.55 (m, 2H), 7.45-7.42 (m, 3H), 2.49 (t, J=7.2 Hz, 2H), 1.85-1.79 (m, 2H), 1.06 (t, J=7.2 Hz, 3H).

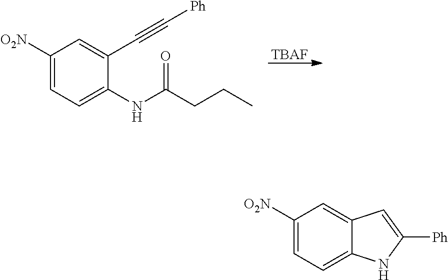 [SMILES: CCCC(=O)Nc1ccc([N+](=O)[O-])cc1C#Cc1ccccc1 ; "TBAF" ; O=[N+]([O-])c1ccc2[nH]c(-c3ccccc3)cc2c1]

### 5-Nitro-2-phenyl-1H-indole

A mixture of N-(2-(phenylethynyl)phenyl)-4-nitrobutyramide (5.0 g, 0.020 mol) and TBAF (12.7 g, 0.050 mol) in THF (30 mL) was heated at reflux for 24 h. The mixture was cooled to room temperature and poured into ice water. The mixture was extracted with CH₂Cl₂ (50 mL×3). The combined organic layers were dried over anhydrous Na₂SO₄ and evaporated under reduced pressure. The residue was purified by column chromatography on silica gel (petroleum ether/ethyl acetate=10/1) to give 5-nitro-2-phenyl-1H-indole (3.3 g, 69%). ¹H NMR (400 MHz, CDCl₃) δ 8.67 (s, 1H), 8.06 (dd, J=2.0, 8.8 Hz, 1H), 7.75 (d, J=7.6 Hz, 2H), 7.54 (d, J=8.8 Hz, 1H), 7.45 (t, J=7.6 Hz, 2H), 7.36 (t, J=7.6 Hz, 1H). 6.95 (s, 1H).

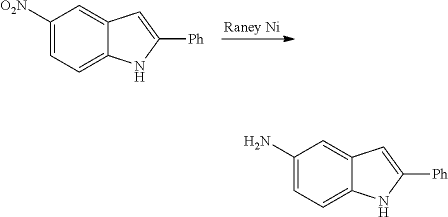

### 2-Phenyl-1H-indol-5-amine

To a solution of 5-nitro-2-phenyl-1H-indole (2.83 g, 0.01 mol) in MeOH (30 mL) was added Raney Ni (510 mg) under nitrogen atmosphere. The mixture was stirred under hydrogen atmosphere (1 atm) at room temperature overnight. The catalyst was filtered through a Celite pad and the filtrate was evaporated under vacuum to give the crude product, which was purified by column chromatography on silica gel (petroleum ether/ethyl acetate=5/1) to give 2-phenyl-1H-indol-5-amine (1.6 g, 77%). ¹H NMR (400 MHz, CDCl₃) δ 7.76 (d, J=7.6 Hz, 2H), 7.39 (t, J=7.6 Hz, 2H), 7.24 (t, J=7.6 Hz, 1H), 7.07 (d, J=8.4 Hz, 1H), 6.64 (d, J=1.6 Hz, 1H), 6.60 (d, J=1.2 Hz, 1H), 6.48 (dd, J=2.0, 8.4 Hz, 1H), 4.48 (brs, 2H); MS (ESI) m/e (M+H⁺) 209.0.

### Example 36

### 2-tert-Butyl-4-fluoro-1H-indol-5-amine

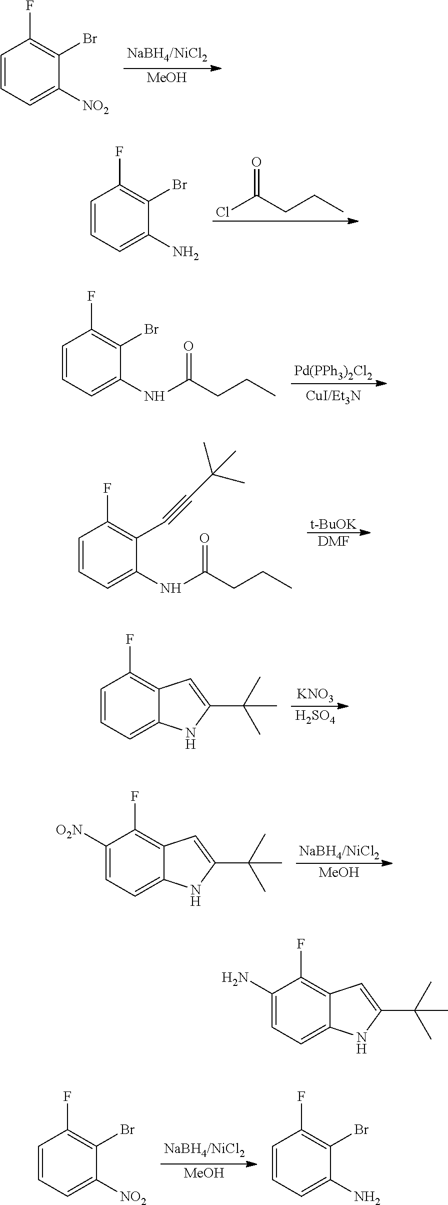

### 2-Bromo-3-fluoroaniline

To a solution of 2-bromo-1-fluoro-3-nitrobenzene (1.0 g, 5.0 mmol) in CH₃OH (50 mL) was added NiCl₂ (2.2 g 10 mmol) and NaBH₄ (0.50 g 14 mmol) at 0° C. After the addition, the mixture was stirred for 5 min. Water (20 mL) was added and the mixture was extracted with EtOAc (20 mL×3). The organic layers were dried over anhydrous Na₂SO₄ and evaporated under vacuum to give 2-bromo-3-fluoroaniline (600 mg, 70%). ¹H NMR (400 MHz, CDCl₃) 7.07-7.02 (m, 1H), 6.55-6.49 (m, 1H), 4.22 (br s, 2H).

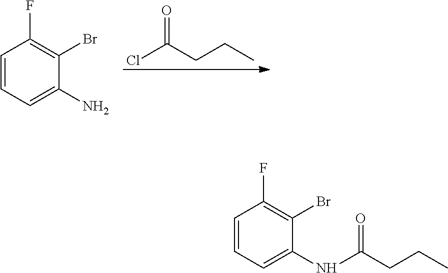

### N-(2-Bromo-3-fluorophenyl)butyramide

To a solution of 2-bromo-3-fluoroaniline (2.0 g, 11 mmol) in CH₂Cl₂ (50 mL) was added butyryl chloride (1.3 g, 13 mmol) and pyridine (1.7 g, 21 mmol) at 0° C. The mixture was stirred at room temperature for 24 h. Water (20 mL) was added and the mixture was extracted with CH₂Cl₂ (50 mL×3). The organic layers were dried anhydrous over Na₂SO₄ and evaporated under vacuum to give N-(2-bromo-3-fluorophenyl)butyramide (2.0 g, 73%), which was directly used in the next step.

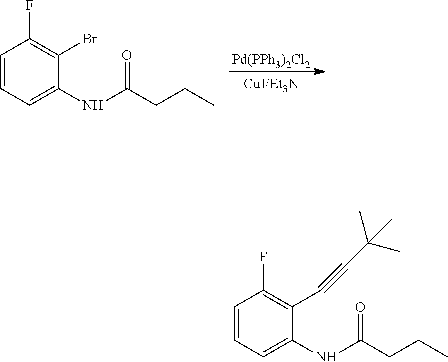 [SMILES: CCCC(=O)Nc1cccc(F)c1Br ; CCCC(=O)Nc1cccc(F)c1C#CC(C)(C)C ; "Pd(PPh3)2Cl2"]

### N-(2-(3,3-Dimethylbut-1-ynyl)-3-fluorophenyl)butyramide

To a solution of N-(2-bromo-3-fluorophenyl)butyramide (2.0 g, 7.0 mmol) in Et₃N (100 mL) was added 4,4-dimethylpent-2-yne (6.0 g, 60 mmol), CuI (70 mg, 3.8 mmol), and Pd(PPh₃)2Cl₂ (500 mg) successively at room temperature under N₂. The mixture was heated at 80° C. overnight. The cooled mixture was filtered and the filtrate was extracted with EtOAc (40 mL×3). The organic layers were washed with sat. NaCl, dried over anhydrous Na₂SO₄, and evaporated under vacuum. The crude compound was purified by column chromatography on silica gel (10% EtOAc in petroleum ether) to give N-(2-(3,3-dimethylbut-1-ynyl)-3-fluorophenyl)butyramide (1.1 g, 55%). ¹H NMR (400 MHz, CDCl₃) 8.20 (d, J=7.6, 1H), 7.95 (s, 1H), 7.21 (m, 1H), 6.77 (t, J=7.6 Hz, 1H), 2.39 (t, J=7.6 Hz, 2H), 1.82-1.75 (m, 2H), 1.40 (s, 9H), 1.12 (t, J=7.2 Hz, 3H).

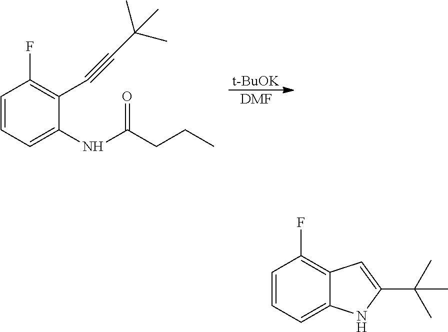

### 2-tert-Butyl-4-fluoro-1H-indole

To a solution of N-(2-(3,3-dimethylbut-1-ynyl)-3-fluorophenyl)butyramide (6.0 g, 20 mmol) in DMF (100 mL) was added t-BuOK (5.0 g, 50 mmol) at room temperature. The mixture was heated at 90° C. overnight before it was poured into water and extracted with EtOAc (100 mL×3). The organic layers were washed with sat. NaCl and water, dried over anhydrous Na₂SO₄, and evaporated under vacuum to give 2-tert-butyl-4-fluoro-1H-indole (5.8 g, 97%). ¹H NMR (400 MHz, CDCl₃) 8.17 (br s, 1H), 7.11 (d, J=7.2 Hz, 1H), 7.05-6.99 (m, 1H), 6.76-6.71 (m, 1H), 6.34 (m, 1H), 1.41 (s, 9H).

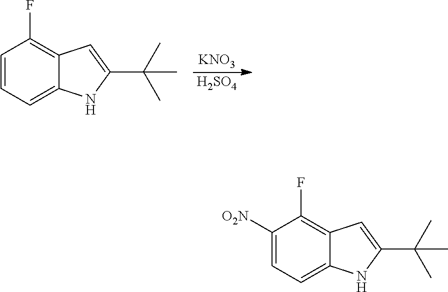

### 2-tert-Butyl-4-fluoro-5-nitro-1H-indole

To a solution of 2-tert-butyl-4-fluoro-1H-indole (2.5 g, 10 mmol) in H₂SO₄ (30 mL) was added KNO₃ (1.3 g, 10 mmol) at 0° C. The mixture was stirred for 0.5 h at −10° C. The mixture was poured into water and extracted with EtOAc (100 mL×3). The organic layers were washed with sat. NaCl and water, dried over anhydrous Na₂SO₄, and evaporated under vacuum. The crude compound was purified by column chromatography on silica gel (10% EtOAc in petroleum ether) to give 2-tert-butyl-4-fluoro-5-nitro-1H-indole (900 mg, 73%). ¹H NMR (400 MHz, CDCl₃) 8.50 (br s, 1H), 7.86 (dd, J=7.6, 8.8 Hz, 1H), 7.13 (d, J=8.8 Hz, 1H), 6.52 (dd, J=0.4, 2.0 Hz, 1H), 1.40 (s, 9H).

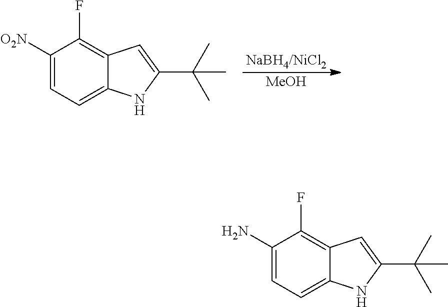

### 2-tert-Butyl-4-fluoro-1H-indol-5-amine

To a solution of 2-tert-butyl-4-fluoro-5-nitro-1H-indole (2.1 g, 9.0 mmol) in methanol (50 mL) was added NiCl₂ (4.2 g, 18 mmol) and NaBH₄ (1.0 g, 27 mmol) at 0° C. After the addition, the mixture was stirred for 5 min. Water (20 mL) was added and the mixture was extracted with EtOAc (30 mL×3). The organic layers were washed with sat. NaCl and water, dried over anhydrous Na₂SO₄, evaporated under vacuum to give 2-tert-butyl-4-fluoro-1H-indol-5-amine (900 mg, 50%). ¹H NMR (300 MHz, CDCl₃) 7.80 (brs, 1H), 6.91 (d, J=8.4 Hz, 1H), 6.64 (dd, J=0.9, 2.4 Hz, 1H), 6.23 (s, 1H), 1.38 (s, 9H).

### Example 37

### 2,3,4,9-Tetrahydro-1H-carbazol-6-amine

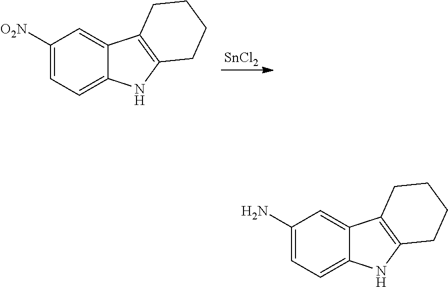

### 2,3,4,9-Tetrahydro-1H-carbazol-6-amine

6-Nitro-2,3,4,9-tetrahydro-1H-carbazole (0.100 g, 0.462 mmol) was dissolved in a 40 mL scintillation vial containing a magnetic stir bar and 2 mL of ethanol. Tin(II) chloride dihydrate (1.04 g, 4.62 mmol) was added to the reaction mixture and the resulting suspension was heated at 70° C. for 16 h. The crude reaction mixture was then diluted with 15 mL of a saturated aqueous solution of sodium bicarbonate and extracted three times with an equivalent volume of ethyl acetate. The ethyl acetate extracts were combined, dried over sodium sulfate, and evaporated to dryness to yield 2,3,4,9-tetrahydro-1H-carbazol-6-amine (82 mg, 95%) which was used without further purification.

### Example 38

### 2-tert-Butyl-7-fluoro-1H-indol-5-amine

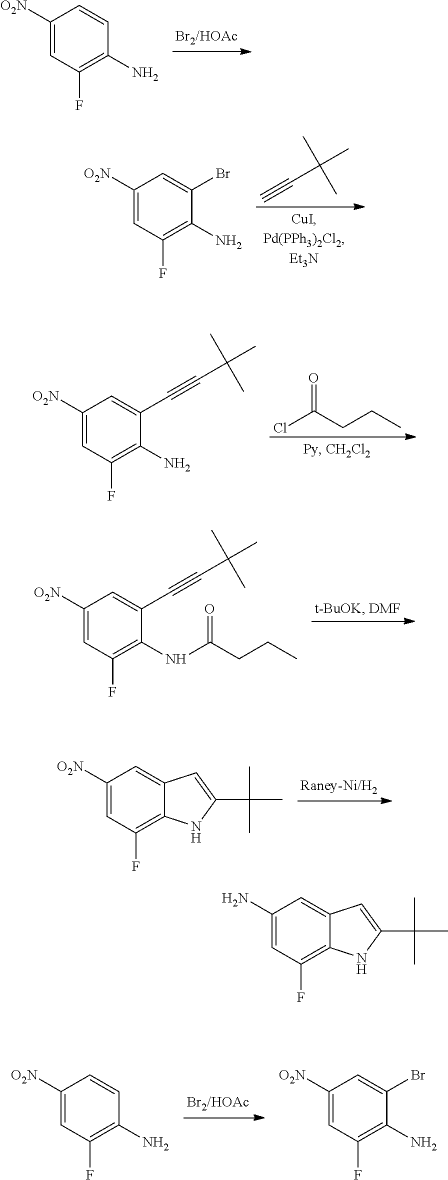

### 2-Bromo-6-fluoro-4-nitro-phenylamine

To a solution of 2-fluoro-4-nitro-phenylamine (12 g, 77 mmol) in AcOH (50 mL) was added Br₂ (3.9 mL, 77 mmol) dropwise at 0° C. The mixture was stirred at 20° C. for 3 h. The reaction mixture was basified with sat. aq. NaHCO₃, and extracted with EtOAc (100 mL×3). The combined organics were dried over anhydrous Na₂SO₄ and evaporated under vacuum to give 2-bromo-6-fluoro-4-nitro-phenylamine (18 g, 97%). ¹H NMR (400 MHz, CDCl₃) δ 8.22 (m, 1H), 7.90 (dd, J=2.4, 10.8 Hz, 1H), 4.88 (brs, 2H).

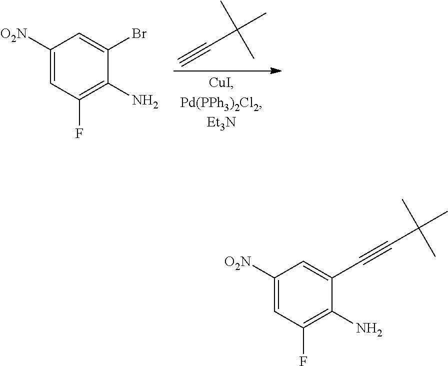

### 2-(3,3-Dimethyl-but-1-ynyl)-6-fluoro-4-nitro-phenylamine

To a solution of 2-bromo-6-fluoro-4-nitro-phenylamine (11 g, 47 mmol) in dry Et₃N (100 mL) was added CuI (445 mg, 5% mol), Pd(PPh₃)2Cl₂ (550 mg, 5% mol) and 3,3-dimethyl-but-1-yne (9.6 g, 120 mmol) under N₂ protection. The mixture was stirred at 80° C. for 10 h. The reaction mixture was filtered, poured into ice (100 g), and extracted with EtOAc (50 mL×3). The combined organic extracts were dried over anhydrous Na₂SO₄ and evaporated under vacuum to give the crude product, which was purified by column chromatography on silica gel (petroleum ether/ethyl acetate 50:1) to give 2-(3,3-dimethyl-but-1-ynyl)-6-fluoro-4-nitro-phenylamine (4.0 g, 36%). ¹H NMR (400 MHz, CDCl₃) δ 8.02 (d, J=1.2 Hz, 1H), 7.84 (dd, J=2.4, 10.8 Hz, 1H), 4.85 (brs, 2H), 1.36 (s, 9H).

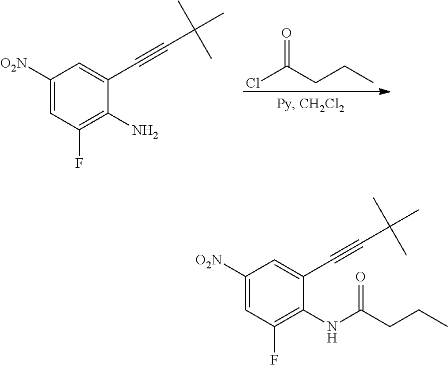

### N-[2-(3,3-Dimethyl-but-1-ynyl)-6-fluoro-4-nitro-phenyl]-butyramide

To a solution of 2-(3,3-dimethyl-but-1-ynyl)-6-fluoro-4-nitro-phenylamine (4.0 g, 17 mmol) and pyridine (2.7 g, 34 mmol) in anhydrous CH₂Cl₂ (30 mL) was added and butyryl chloride (1.8 g, 17 mmol) dropwise at 0° C. After stirring for 5 h at 0° C., the reaction mixture was poured into ice (50 g) and extracted with CH₂Cl₂ (30 mL×3). The combined organic extracts were dried over anhydrous Na₂SO₄ and evaporated under vacuum to give N-[2-(3,3-dimethyl-but-1-ynyl)-6-fluoro-4-nitro-phenyl]-butyramide (3.2 g, 62%), which was used in the next step without further purification. ¹H NMR (300 MHz, DMSO) δ 8.10 (dd, J=1.5, 2.7 Hz, 1H), 7.95 (dd, J=2.4, 9.6 Hz, 1H), 7.22 (brs, 1H), 2.45 (t, J=7.5 Hz, 2H), 1.82 (m, 2H), 1.36 (s, 9H), 1.06 (t, J=7.5 Hz, 3H).

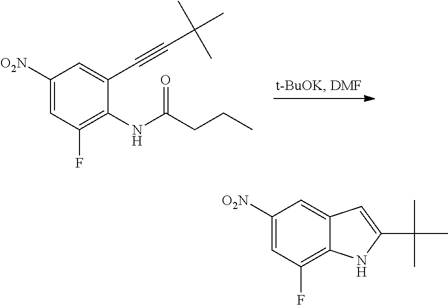 [SMILES: CCCC(=O)Nc1c(F)cc([N+](=O)[O-])cc1C#CC(C)(C)C ; CC(C)(C)c1cc2cc([N+](=O)[O-])cc(F)c2[nH]1]

### 2-tert-Butyl-7-fluoro-5-nitro-1H-indole

To a solution of N-[2-(3,3-dimethyl-but-1-ynyl)-6-fluoro-4-nitro-phenyl]-butyramide (3.2 g, 10 mmol) in DMF (20 mL) was added t-BuOK (2.3 g, 21 mmol) at room temperature. The mixture was heated at 120° C. for 2 g before being cooled down to room temperature. Water (50 mL) was added to the reaction mixture and the resulting mixture was extracted with CH₂Cl₂ (30 mL×3). The combined organic extracts were dried over anhydrous Na₂SO₄ and evaporated under vacuum to give 2-tert-butyl-7-fluoro-5-nitro-1H-indole (2.0 g, 81%), which was used in the next step without further purification. ¹H NMR (300 MHz, CDCl₃) δ 9.95 (brs, 1H), 8.30 (d, J=2.1 Hz, 1H), 7.74 (dd, J=1.8, 11.1 Hz, 1H), 6.43 (dd, J=2.4, 3.3 Hz, 1H), 1.43 (s, 9H).

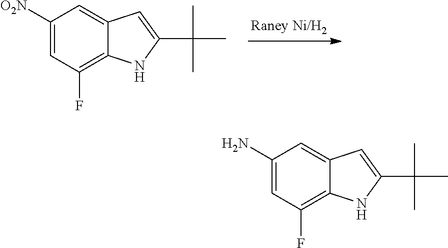 [SMILES: CC(C)(C)c1cc2cc([N+](=O)[O-])cc(F)c2[nH]1 ; CC(C)(C)c1cc2cc(N)cc(F)c2[nH]1]

### 2-tert-Butyl-7-fluoro-1H-indol-5-amine

To a solution of 2-tert-butyl-7-fluoro-5-nitro-1H-indole (2.0 g, 8.5 mmol) in MeOH (20 mL) was added Ni (0.3 g) under nitrogen atmosphere. The reaction mixture was stirred under hydrogen atmosphere (1 atm) at room temperature overnight. The catalyst was filtered off through the celite pad and the filtrate was evaporated under vacuum. The crude product was purified by column chromatography on silica gel (petroleum ether/ethyl acetate 100:1) to give 2-tert-butyl-7-fluoro-1H-indol-5-amine (550 mg, 24%). ¹H NMR (300 MHz, CDCl₃) δ 7.87 (brs, 1H), 6.64 (d, J=1.5 Hz, 1H), 6.37 (dd, J=1.8, 12.3 Hz, 1H), 6.11 (dd, J=2.4, 3.6 Hz, 1H), 1.39 (s, 9H). MS (ESI) m/z (M+H⁺) 207.

### Example 39

### 5-Amino-2-tert-butyl-1H-indole-7-carbonitrile

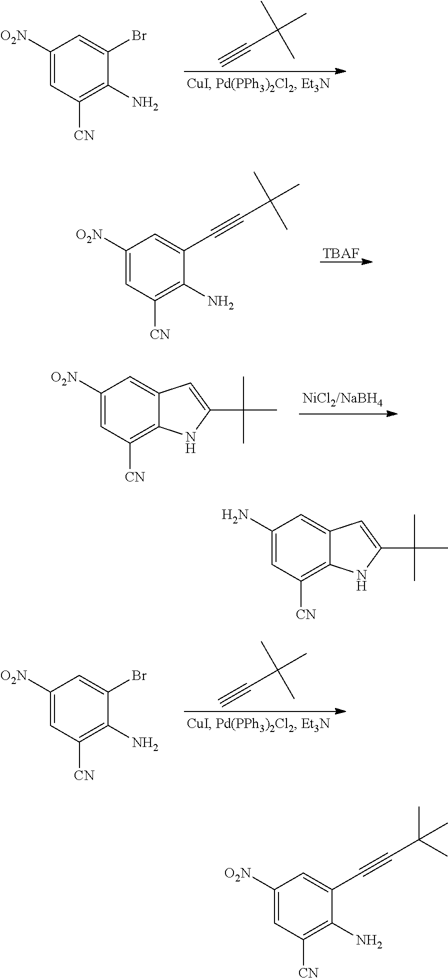

### 2-Amino-3-(3,3-dimethylbut-1-ynyl)-5-nitrobenzonitrile

To a stirred solution of 2-amino-3-bromo-5-nitrobenzonitrile (2.4 g, 10 mmol) in dry Et₃N (60 mL) was added CuI (380 mg, 5% mol) and Pd(PPh₃)2Cl₂ (470 mg, 5% mol) at room temperature. 3,3-dimethyl-but-1-yne (2.1 g, 25 mmol) was added dropwise to the mixture at room temperature. The reaction mixture was stirred at 80° C. for 10 h. The reaction mixture was filtered and the filtrate was poured into ice (60 g), extracted with ethyl acetate. The phases were separated and the organic phase was dried over Na₂SO₄. The solvent was removed under vacuum to obtain the crude product, which was purified by column chromatography (2-10% EtOAc in petroleum ether) to obtain 2-amino-3-(3,3-dimethylbut-1-ynyl)-5-nitrobenzonitrile (1.7 g, 71%). ¹H NMR (300 MHz, CDCl₃) δ 8.28 (d, J=2.7 Hz, 1H), 8.27 (d, J=2.7 Hz, 1H), 5.56 (br s, 2H), 1.37 (s, 9H).

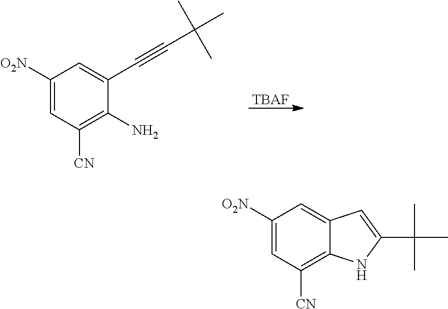

### 2-tert-Butyl-5-nitro-1H-indole-7-carbonitrile

To a solution of 2-amino-3-(3,3-dimethylbut-1-ynyl)-5-nitrobenzonitrile (1.7 g, 7.0 mmol) in THF (35 mL) was added TBAF (9.5 g, 28 mmol) at room temperature. The mixture was heated at reflux overnight. The reaction mixture was cooled and the THF was removed under reduced pressure. Water (50 ml) was added to the residue and the mixture was extracted with EtOAc. The organics were dried over Na₂SO₄ and the solvent was evaporated under vacuum to obtain 0.87 g of crude product 2-tert-butyl-5-nitro-1H-indole-7-carbonitrile which was used directly in the next step without purification.

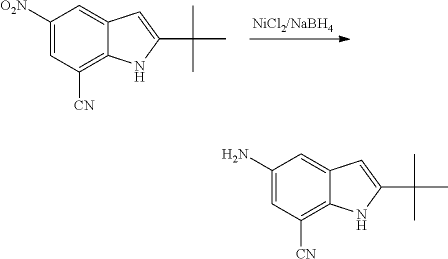 [SMILES: CC(C)(C)c1cc2cc([N+](=O)[O-])cc(C#N)c2[nH]1 ; CC(C)(C)c1cc2cc(N)cc(C#N)c2[nH]1]

### 5-Amino-2-tert-butyl-1H-indol-7-carbonitrile

To a solution of crude product 2-tert-butyl-5-nitro-1H-indole-7-carbonitrile (0.87 g, 3.6 mmol) in MeOH (10 mL) was added NiCl₂.6H₂O (1.8 g, 7.2 mmol) at −5° C. The reaction mixture was stirred for 30 min, then NaBH₄ (0.48 g, 14.32 mmol) was added to the reaction mixture at 0° C. After 5 min, the reaction mixture was quenched with water, filtered and extracted with EtOAc. The combined organic layers were dried over Na₂SO₄ and concentrated under vacuum to obtain the crude product, which was purified by column chromatography (5-20% EtOAc in petroleum ether) to obtain 5-amino-2-tert-butyl-1H-indol-7-carbonitrile (470 mg, 32% over two steps). ¹H NMR (400 MHz, CDCl₃) δ 8.25 (s, 1H), 7.06 (d, J=2.4 Hz, 1H), 6.84 (d, J=2.4 Hz, 1H), 6.14 (d, J=2.4 Hz, 1H), 3.57 (br s, 2H), 1.38 (s, 9H). MS (ESI) m/z: 214 (M+H⁴).

### Example 40

### Methyl 5-amino-2-tert-butyl-1H-indole-7-carboxylate

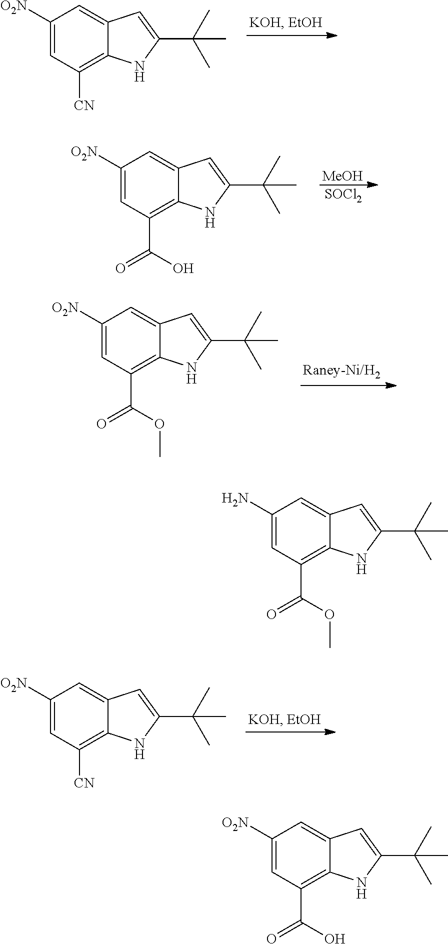

### 2-tert-Butyl-5-nitro-1H-indole-7-carboxylic acid

2-tert-Butyl-5-nitro-1H-indole-7-carbonitrile (4.6 g, 19 mmol) was added to a solution of KOH in EtOH (10%, 100 mL) and the mixture was heated at reflux overnight. The solution was evaporated to remove alcohol, a small amount of water was added, and then the mixture was acidified with dilute hydrochloric acid. Upon standing in the refrigerator, an orange-yellow solid precipitated, which was purified by chromatography on silica gel (15% EtOAc in petroleum ether) to afford 2-tert-butyl-5-nitro-1H-indole-7-carboxylic acid (4.0 g, 77%). ¹H NMR (CDCl₃, 300 MHz) δ 10.79 (brs, 1H), 8.66 (s, 1H), 8.45 (s, 1H), 6.57 (s, 1H), 1.39 (s, 9H).

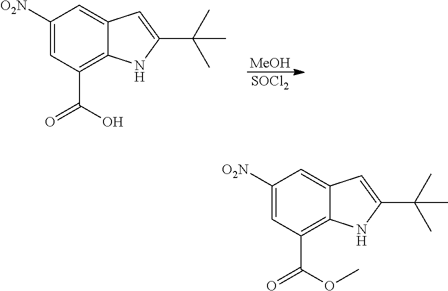

### Methyl 2-tert-butyl-5-nitro-1H-indole-7-carboxylate

SOCl₂ (3.6 g, 30 mol) was added dropwise to a solution of 2-tert-butyl-5-nitro-1H-indole-7-carboxylic acid (4.0 g, 15 mol) and methanol (30 mL) at 0° C. The mixture was stirred at 80° C. for 12 h. The solvent was evaporated under vacuum and the residue was purified by column chromatography on silica gel (5% EtOAc in petroleum ether) to afford methyl 2-tert-butyl-5-nitro-1H-indole-7-carboxylate (2.95 g, 70%). ¹H NMR (CDCl₃, 300 MHz) δ 9.99 (brs, 1H), 8.70 (d, J=2.1 Hz, 1H), 8.65 (d, J=2.1 Hz, 1H), 6.50 (d, J=2.4 Hz, 1H), 4.04 (s, 3H), 1.44 (s, 9H).

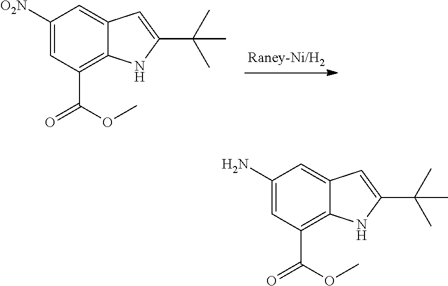 [SMILES: COC(=O)c1cc([N+](=O)[O-])cc2cc(C(C)(C)C)[nH]c12 ; COC(=O)c1cc(N)cc2cc(C(C)(C)C)[nH]c12]

### Methyl 5-amino-2-tert-butyl-1H-indole-7-carboxylate

A solution of 2-tert-butyl-5-nitro-1H-indole-7-carboxylate (2.0 g, 7.2 mmol) and Raney Nickel (200 mg) in CH₃OH (50 mL) was stirred for 5 h at the room temperature under H₂ atmosphere. The catalyst was filtered off through a celite pad and the filtrate was evaporated under vacuum to give methyl 5-amino-2-tert-butyl-1H-indole-7-carboxylate (1.2 g, 68%) ¹H NMR (CDCl₃, 400 MHz) δ 9.34 (brs, 1H), 7.24 (d, J=1.6 Hz, 1H), 7.10 (s, 1H), 6.12 (d, J=1.6 Hz, 1H), 3.88 (s, 3H), 1.45 (s, 9H).

### Example 41

### (5-Amino-2-tert-butyl-1H-indol-7-yl)methanol

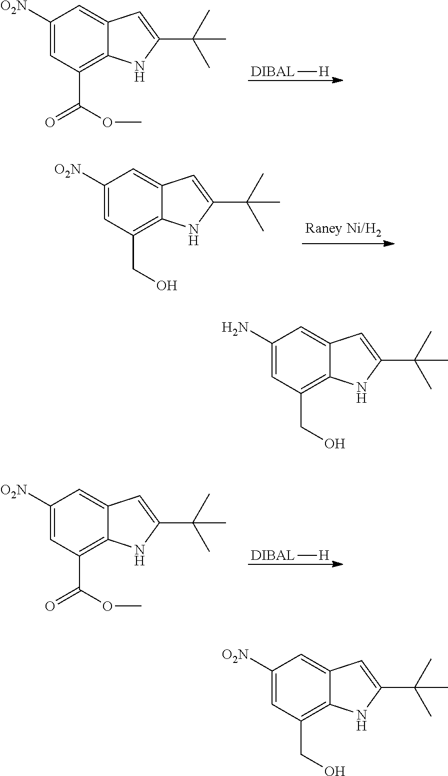

### (2-tert-Butyl-5-nitro-1H-indol-7-yl)methanol

To a solution of methyl 2-tert-butyl-5-nitro-1H-indole-7-carboxylate (6.15 g, 22.3 mmol) and dichloromethane (30 ml) was added DIBAL-H (1.0 M, 20 mL, 20 mmol) at 78° C. The mixture was stirred for 1 h before water (10 mL) was added slowly. The resulting mixture was extracted with EtOAc (120 mL×3). The combined organic extracts were dried over anhydrous Na₂SO₄ and evaporated under vacuum to give (2-tert-butyl-5-nitro-1H-indol-7-yl)methanol (4.0 g, 73%), which was used in the next step directly.

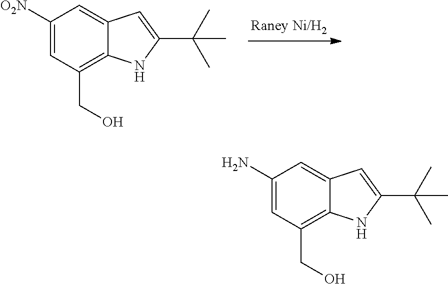 [SMILES: CC(C)(C)c1cc2cc([N+](=O)[O-])cc(CO)c2[nH]1 ; CC(C)(C)c1cc2cc(N)cc(CO)c2[nH]1]

### (5-Amino-2-tert-butyl-1H-indol-7-yl)methanol

A mixture of (2-tert-butyl-5-nitro-1H-indol-7-yl)methanol (4.0 g, 16 mmol) and Raney Nickel (400 mg) in CH₃OH (100 mL) was stirred for 5 g at room temperature under H₂. The catalyst was filtered off through a celite pad and the filtrate was evaporated under vacuum to give (5-amino-2-tert-butyl-1H-indol-7-yl)methanol (3.4 g, 80%). ¹H NMR (CDCl₃, 400 MHz) δ 8.53 (br s, 1H), 6.80 (d, J=2.0 Hz, 1H), 6.38 (d, J=1.6 Hz, 1H), 4.89 (s, 2H), 1.37 (s, 9H).

### Example 42

### 2-(1-Methylcyclopropyl)-1H-indol-5-amine

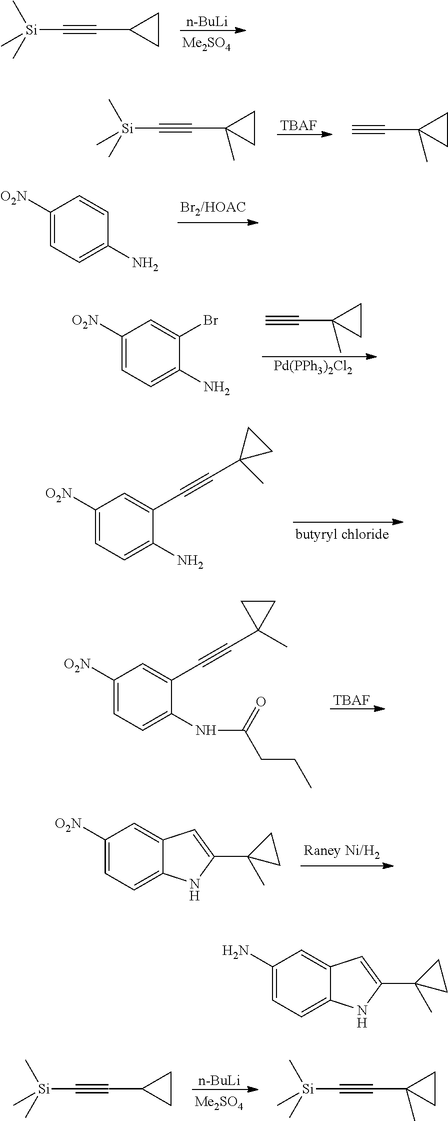

### Trimethyl-(1-methyl-cyclopropylethynyl)-silane

To a solution of cyclopropylethynyl-trimethyl-silane (3.0 g, 22 mmol) in ether (20 mL) was added dropwise n-BuLi (8.6 mL, 21.7 mol, 2.5 M solution in hexane) at 0° C. The reaction mixture was stirred at ambient temperature for 24 h before dimethyl sulfate (6.85 g, 54.3 mmol) was added dropwise at −10° C. The resulting solution was stirred at 10° C. and then at 20° C. for 30 min each. The reaction was quenched by adding a mixture of sat. aq. NH₄Cl and 25% aq. ammonia (1:3, 100 mL). The mixture was then stirred at ambient temperature for 1 h. The aqueous phase was extracted with diethyl ether (3×50 mL) and the combined organic layers were washed successively with 5% aqueous hydrochloric acid (100 mL), 5% aq.

NaHCO₃ solution (100 mL), and water (100 mL). The organics were dried over anhydrous NaSO₄ and concentrated at ambient pressure. After fractional distillation under reduced pressure, trimethyl-(1-methyl-cyclopropylethynyl)-silane (1.7 g, 52%) was obtained as a colorless liquid. ¹H NMR (400 MHz, CDCl₃) δ 1.25 (s, 3H), 0.92-0.86 (m, 2H), 0.58-0.56 (m, 2H), 0.15 (s, 9H).

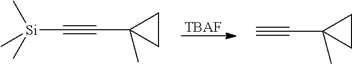

### 1-Ethynyl-1-methyl-cyclopropane

To a solution of trimethyl-(1-methyl-cyclopropylethynyl)-silane (20 g, 0.13 mol) in THF (250 mL) was added TBAF (69 g, 0.26 mol). The mixture was stirred overnight at 20° C. The mixture was poured into water and the organic layer was separated. The aqueous phase was extracted with THF (50 mL). The combined organic layers were dried over anhydrous Na₂SO₄ and distilled under atmospheric pressure to obtain 1-ethynyl-1-methyl-cyclopropane (7.0 g, contained ½ THF, 34%). ¹H NMR (400 MHz, CDCl₃) δ 1.82 (s, 1H), 1.26 (s, 3H), 0.90-0.88 (m, 2H), 0.57-0.55 (m, 2H).

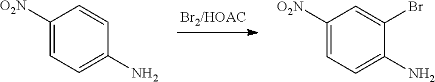 [SMILES: Nc1ccc([N+](=O)[O-])cc1 ; Nc1ccc([N+](=O)[O-])cc1Br]

### 2-Bromo-4-nitroaniline

To a solution of 4-nitro-phenylamine (50 g, 0.36 mol) in AcOH (500 mL) was added Br₂ (60 g, 0.38 mol) dropwise at 5° C. The mixture was stirred for 30 min at that temperature. The insoluble solid was collected by filtration and basified with saturated aqueous NaHCO₃ to pH 7. The aqueous phase was extracted with EtOAc (300 mL×3). The combined organic layers were dried and evaporated under reduced pressure to obtain compound 2-bromo-4-nitroaniline (56 g, 72%), which was directly used in the next step.

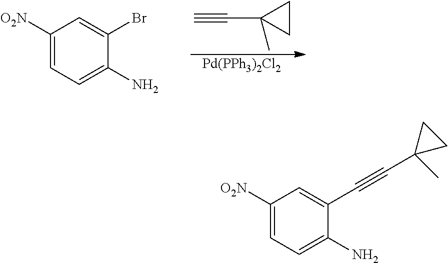

### 2-((1-Methylcyclopropyl)ethynyl)-4-nitroaniline

To a deoxygenated solution of 2-bromo-4-nitroaniline (430 mg, 2.0 mmol) and 1-ethynyl-1-methyl-cyclopropane (630 mg, 8.0 mmol) in triethylamine (20 mL) was added CuI (76 mg, 0.40 mmol) and Pd(PPh₃)2Cl₂ (140 mg, 0.20 mmol) under N₂. The mixture was heated at 70° C. and stirred for 24 h. The solid was filtered off and washed with EtOAc (50 mL×3). The filtrate was evaporated under reduced pressure and the residue was purified by column chromatography on silica gel (petroleum ether/ethyl acetate=10/1) to give 2-((1-methylcyclopropyl)ethynyl)-4-nitroaniline (340 mg, 79%). ¹H NMR (300 MHz, CDCl₃) δ 8.15-8.14 (m, 1H), 7.98-7.95 (m, 1H), 6.63 (d, J=6.9 Hz, 1H), 4.80 (brs, 2H), 1.38 (s, 3H), 1.04-1.01 (m, 2H), 0.76-0.73 (m, 2H).

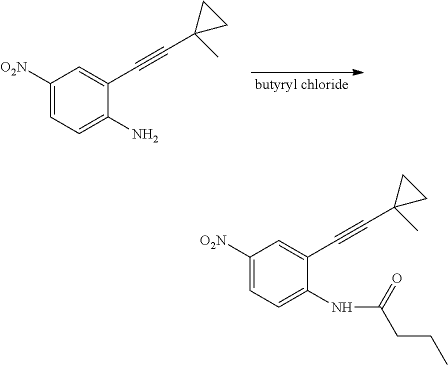 [SMILES: CC1(C#Cc2cc([N+](=O)[O-])ccc2N)CC1 ; CCCC(=O)Nc1ccc([N+](=O)[O-])cc1C#CC1(C)CC1]

### N-[2-(1-Methyl-cyclopropylethynyl)-4-nitro-phenyl]-butyramide

To a solution of 2-((1-methylcyclopropyl)ethynyl)-4-nitroaniline (220 mg, 1.0 mmol) and pyridine (160 mg, 2.0 mol) in CH₂Cl₂ (20 mL) was added butyryl chloride (140 mg, 1.3 mmol) at 0° C. The mixture was warmed to room temperature and stirred for 3 h. The mixture was poured into ice-water. The organic layer was separated and the aqueous phase was extracted with CH₂Cl₂ (30 mL×3). The combined organic layers were dried over anhydrous Na₂SO₄ and evaporated under reduced pressure to obtain N-[2-(1-methyl-cyclopropyl-ethynyl)-4-nitro-phenyl]-butyramide (230 mg, 82%), which was directly used in the next step.

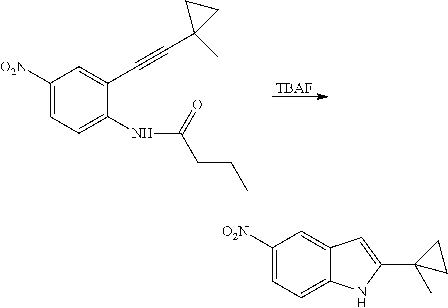

### 2-(1-Methylcyclopropyl)-5-nitro-1H-indole

A mixture of N-[2-(1-methyl-cyclopropylethynyl)-4-nitro-phenyl]-butyramide (1.3 g, 4.6 mmol) and TBAF (2.4 g, 9.2 mmol) in THF (20 mL) was heated at reflux for 24 h. The mixture was cooled to room temperature and poured into ice water. The mixture was extracted with CH₂Cl₂ (30 mL×3). The combined organic layers were dried over anhydrous Na₂SO₄ and evaporated under reduced pressure. The residue was purified by column chromatography on silica gel (petroleum ether/ethyl acetate=10/1) to afford 2-(1-methylcyclopropyl)-5-nitro-1H-indole (0.70 g, 71%). ¹H NMR (400 MHz, CDCl₃) δ 8.56 (brs, 1H), 8.44 (d, J=2.0 Hz, 1H), 8.01 (dd, J=2.4, 8.8 Hz, 1H), 7.30 (d, J=8.8 Hz, 1H), 6.34 (d, J=1.6 Hz, 1H), 1.52 (s, 3H), 1.03-0.97 (m, 2H), 0.89-0.83 (m, 2H).

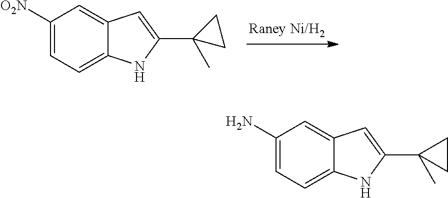 [SMILES: CC1(c2cc3cc([N+](=O)[O-])ccc3[nH]2)CC1 ; CC1(c2cc3cc(N)ccc3[nH]2)CC1]

### 2-(1-Methyl-cyclopropyl)-1H-indol-5-ylamine

To a solution of 2-(1-methylcyclopropyl)-5-nitro-1H-indole (0.70 g, 3.2 mmol) in EtOH (20 mL) was added Raney Nickel (100 mg) under nitrogen atmosphere. The mixture was stirred under hydrogen atmosphere (1 atm) at room temperature overnight. The catalyst was filtered off through a celite pad and the filtrate was evaporated under vacuum. The residue was purified by column chromatography on silica gel (petroleum ether/ethyl acetate=5/1) to afford 2-(1-methyl-cyclopropyl)-1H-indol-5-ylamine (170 mg, 28%). ¹H NMR (400 MHz, CDCl₃) δ 7.65 (brs, 1H), 7.08 (d, J=8.4 Hz, 1H), 6.82 (s, 1H), 6.57 (d, J=8.4 Hz, 1H), 6.14 (s, 1H), 3.45 (brs, 2H), 1.47 (s, 3H), 0.82-0.78 (m, 2H), 0.68-0.63 (m, 2H).

### Example 43

### Methyl 2-(5-amino-1H-indol-2-yl)-2-methylpropanoate

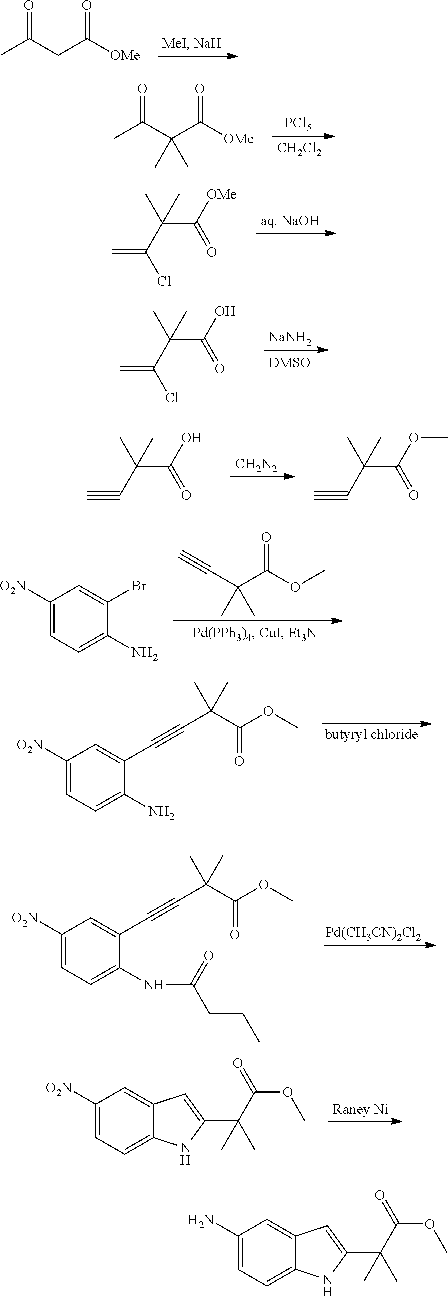

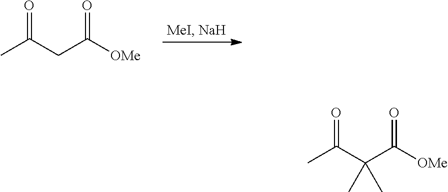

### Methyl 2,2-dimethyl-3-oxobutanoate

To a suspension of NaH (42 g, 1.1 mol, 60%) in THF (400 mL) was added dropwise a solution of methyl 3-oxobutanoate (116 g, 1.00 mol) in THF (100 mL) at 0° C. The mixture was stirred for 0.5 h at that temperature before MeI (146 g, 1.1 mol) was added dropwise at 0° C. The resultant mixture was warmed to room temperature and stirred for 1 h. NaH (42 g, 1.05 mol, 60%) was added in portions at 0° C. and the resulting mixture was continued to stir for 0.5 h at this temperature. MeI (146 g, 1.05 mol) was added dropwise at 0° C. The reaction mixture was warmed to room temperature and stirred overnight. The mixture was poured into ice water and the organic layer was separated. The aqueous phase was extracted with EtOAc (500 mL×3). The combined organic layers were dried and evaporated under reduced pressure to give methyl 2,2-dimethyl-3-oxobutanoate (85 g), which was used directly in the next step.

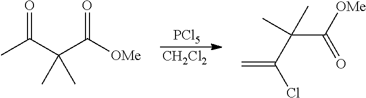

### Methyl 3-chloro-2,2-dimethylbut-3-enoate

To a suspension of PCl₅ (270 g, 1.3 mol) in CH₂Cl₂ (1000 mL) was added dropwise methyl 2,2-dimethyl-3-oxobutanoate (85 g) at 0° C., following by addition of approximately 30 drops of dry DMF. The mixture was heated at reflux overnight. The reaction mixture was cooled to ambient temperature and slowly poured into ice water. The organic layer was separated and the aqueous phase was extracted with CH₂Cl₂ (500 mL×3). The combined organic layers were washed with saturated aqueous NaHCO₃ and dried over anhydrous Na₂SO₄. The solvent was evaporated and the residue was distilled under reduced pressure to give methyl 3-chloro-2,2-dimethylbut-3-enoate (37 g, 23%). ¹H NMR (400 MHz, CDCl₃) δ 5.33 (s, 1H), 3.73 (s, 3H), 1.44 (s, 6H).

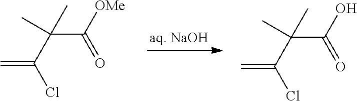

### 3-Chloro-2,2-dimethylbut-3-enoic acid

A mixture of methyl 3-chloro-2,2-dimethylbut-3-enoate (33 g, 0.2 mol) and NaOH (9.6 g, 0.24 mol) in water (200 mL) was heated at reflux for 5 h. The mixture was cooled to ambient temperature and extracted with ether. The organic layer was discarded. The aqueous layer was acidified with cold 20% HCl solution and extracted ether (200 mL×3). The combined organic layers were dried and evaporated under reduced pressure to give 3-chloro-2,2-dimethyl-but-3-enoic acid (21 g, 70%), which was used directly in the next step. ¹H NMR (400 MHz, CDCl₃) δ 7.90 (brs, 1H), 5.37 (dd, J=2.4, 6.8 Hz, 2H), 1.47 (s, 6H).

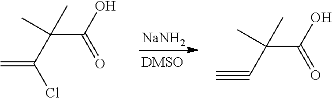

### 2,2-Dimethyl-but-3-ynoic acid

Liquid NH₃ was condensed in a 3-neck, 250 mL round bottom flask at −78° C. Na (3.98 g, 0.173 mol) was added to the flask in portions. The mixture was stirred for 2 h at −78° C. before anhydrous DMSO (20 mL) was added dropwise at −78° C. The mixture was stirred at room temperature until no more NH₃ was given off. A solution of 3-chloro-2,2-dimethyl-but-3-enoic acid (6.5 g, 43 mmol) in DMSO (10 mL) was added dropwise at −40° C. The mixture was warmed and stirred at 50° C. for 5 h, then stirred at room temperature overnight. The cloudy, olive green solution was poured into cold 20% HCl solution and then extracted three times with ether. The ether extracts were dried over anhydrous Na₂SO₄ and concentrated to give crude 2,2-dimethyl-but-3-ynoic acid (2 g), which was used directly in the next step. ¹H NMR (400 MHz, CDCl₃) δ 2.30 (s, 1H), 1.52 (s, 6H).

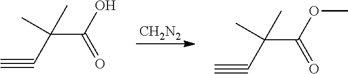

### Methyl 2,2-dimethylbut-3-ynoate

To a solution of diazomethane (˜10 g) in ether (400 mL) was added dropwise 2,2-dimethyl-but-3-ynoic acid (10.5 g, 93.7 mmol) at 0° C. The mixture was warmed to room temperature and stirred overnight. The mixture was distilled under atmospheric pressure to give crude methyl 2,2-dimethylbut-3-ynoate (14 g), which was used directly in the next step. ¹H NMR (400 MHz, CDCl₃) δ 3.76 (s, 3H), 2.28 (s, 1H), 1.50 (s, 6H).

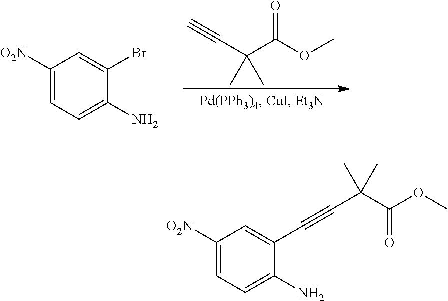

### Methyl 4-(2-amino-5-nitrophenyl)-2,2-dimethylbut-3-ynoate

To a deoxygenated solution of compound 2-bromo-4-nitroaniline (9.43 g, 43.7 mmol), methyl 2,2-dimethylbut-3-ynoate (5.00 g, 39.7 mmol), CuI (754 mg, 3.97 mmol) and triethylamine (8.03 g, 79.4 mmol) in toluene/H₂O (100/30 mL) was added Pd(PPh₃)₄ (6.17 g, 3.97 mmol) under N₂. The mixture was heated at 70° C. and stirred for 24 h. After cooling, the solid was filtered off and washed with EtOAc (50 mL×3). The organic layer was separated and the aqueous phase was washed with EtOAc (50 mL×3). The combined organic layers were dried and evaporated under reduced pressure to give a residue, which was purified by column chromatography on silica gel (petroleum ether/ethyl acetate=10/1) to obtain methyl 4-(2-amino-5-nitrophenyl)-2,2-dimethylbut-3-ynoate (900 mg, 9%). ¹H NMR (400 MHz, CDCl₃) δ 8.17 (d, J=2.8 Hz, 1H), 8.01 (dd, J=2.8, 9.2 Hz, 1H), 6.65 (d, J=9.2 Hz, 1H), 5.10 (brs, 2H), 3.80 (s, 3H), 1.60 (s, 6H).

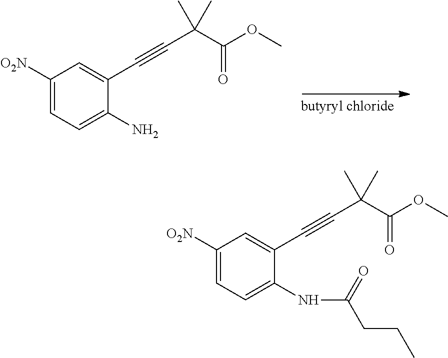 [SMILES: COC(=O)C(C)(C)C#Cc1cc([N+](=O)[O-])ccc1N ; CCCC(=O)Nc1ccc([N+](=O)[O-])cc1C#CC(C)(C)C(=O)OC]

### Methyl 4-(2-butyramido-5-nitrophenyl)-2,2-dimethylbut-3-ynoate

To a solution of methyl 4-(2-amino-5-nitrophenyl)-2,2-dimethylbut-3-ynoate (260 mg, 1.0 mmol) and pyridine (160 mg, 2.0 mol) in CH₂Cl₂ (20 mL) was added butyryl chloride (140 mg, 1.3 mmol) at 0° C. The reaction mixture was warmed to room temperature and stirred for 3 h before the mixture was poured into ice-water. The organic layer was separated and the aqueous phase was extracted with CH₂Cl₂ (30 mL×3). The combined organic layers were dried over anhydrous Na₂SO₄ and evaporated under reduced pressure to obtain methyl 4-(2-butyramido-5-nitrophenyl)-2,2-dimethylbut-3-ynoate (150 mg, 45%), which was used directly in the next step. ¹H NMR (400 MHz, CDCl₃) δ 8.79 (brs, 1H), 8.71 (d, J=9.2 Hz, 1H), 8.24 (d, J=2.8 Hz, 1H), 8.17 (dd, J=2.8, 9.2 Hz, 1H), 3.82 (s, 3H), 2.55 (t, J=7.2 Hz, 2H), 1.85-1.75 (m, 2H), 1.63 (s, 6H), 1.06 (t, J=6.8 Hz, 3H).

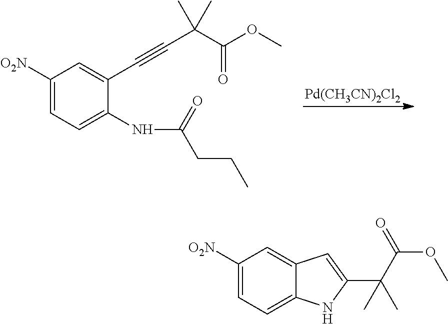

Methyl 2-methyl-2-(5-nitro-1H-indol-2-yl)propanoate

To a deoxygenated solution of methyl 4-(2-butyramido-5-nitrophenyl)-2,2-dimethylbut-3-ynoate (1.8 g, 5.4 mmol) in acetonitrile (30 mL) was added Pd(CH₃CN)2Cl₂ (0.42 g, 1.6=mmol) under N₂. The mixture was heated at reflux for 24 h. After cooling the mixture to ambient temperature, the solid was filtered off and washed with EtOAc (50 mL×3). The filtrate was evaporated under reduced pressure to give a residue, which was purified by column chromatography on silica gel (petroleum ether/ethyl acetate=30/1) to give methyl 2-methyl-2-(5-nitro-1H-indol-2-yl) propanoate (320 mg, 23%). ¹H NMR (400 MHz, CDCl₃) δ 9.05 (brs, 1H), 8.52 (d, J=2.0 Hz, 1H), 8.09 (dd, J=2.0, 8.8 Hz, 1H), 7.37 (d, J=8.8 Hz, 1H), 6.54 (d, J=1.6 Hz, 1H), 3.78 (d, J=9.6 Hz, 3H), 1.70 (s, 6H).

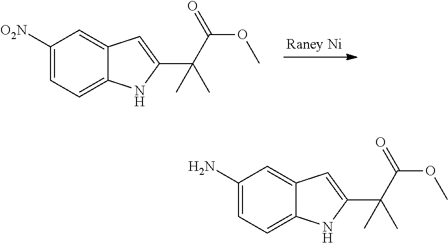 [SMILES: COC(=O)C(C)(C)c1cc2cc([N+](=O)[O-])ccc2[nH]1 ; COC(=O)C(C)(C)c1cc2cc(N)ccc2[nH]1]

### Methyl 2-(5-amino-1H-indol-2-yl)-2-methylpropanoate

A suspension of methyl 2-methyl-2-(5-nitro-1H-indol-2-yl)propanoate (60 mg, 0.23 mmol) and Raney Nickel (10 mg) in MeOH (5 mL) was hydrogenated under hydrogen (1 atm) at room temperature overnight. The catalyst was filtered off through a celite pad and the filtrate was evaporated under vacuum to give a residue, which was purified by column chromatography on silica gel (petroleum ether/ethyl acetate=5/1) to give methyl 2-(5-amino-1H-indol-2-yl)-2-methylpropanoate (20 mg, 38%). ¹H NMR (400 MHz, CDCl₃) δ 8.37 (br s, 1H), 7.13 (d, J=8.4 Hz, 1H), 6.87 (d, J=2.0 Hz, 1H), 6.63 (dd, J=2.0, 8.4 Hz, 1H), 6.20 (d, J=1.2 Hz, 1H), 3.72 (d, J=7.6 Hz, 3H), 3.43 (br s, 1H), 1.65 (s, 6H); MS (ESI) m/e (M+H⁺) 233.2.

### Example 44

### 2-Isopropyl-1H-indol-5-amine

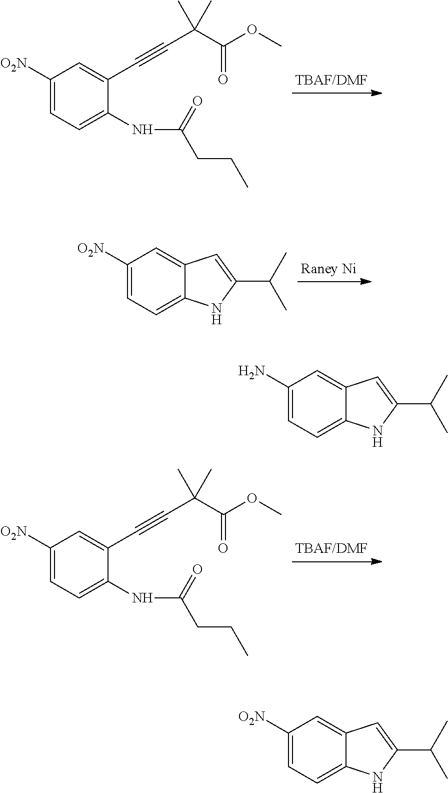 [SMILES: CCCC(=O)Nc1ccc([N+](=O)[O-])cc1C#CC(C)(C)C(=O)OC ; CC(C)c1cc2cc([N+](=O)[O-])ccc2[nH]1 ; CC(C)c1cc2cc(N)ccc2[nH]1 ; CCCC(=O)Nc1ccc([N+](=O)[O-])cc1C#CC(C)(C)C(=O)OC ; CC(C)c1cc2cc([N+](=O)[O-])ccc2[nH]1]

### 2-Isopropyl-5-nitro-1H-indole

A mixture of methyl 4-(2-butyramido-5-nitrophenyl)-2,2-dimethylbut-3-ynoate (0.50 g, 1.5 mmol) and TBAF (790 mg, 3.0 mmol) in DMF (20 mL) was heated at 70° C. for 24 h. The reaction mixture was cooled to room temperature and poured into ice water. The mixture was extracted with ether (30 mL×3). The combined organic layers were dried over anhydrous Na₂SO₄ and evaporated under reduced pressure to give a residue, which was purified by column chromatography on silica gel (petroleum ether/ethyl acetate=20/1) to give 2-isopropyl-5-nitro-1H-indole (100 mg, 33%). ¹H NMR (400 MHz, CDCl₃) δ 8.68 (s, 1H), 8.25 (br s, 1H), 8.21 (dd, J=2.4, 10.0 Hz, 1H), 7.32 (d, J=8.8 Hz, 1H), 6.41 (s, 1H), 3.07-3.14 (m, 1H), 1.39 (d, J=6.8 Hz, 6H).

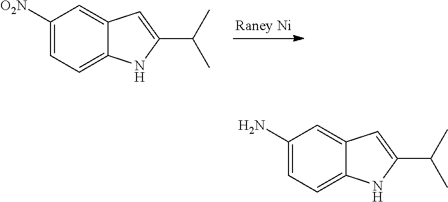

### 2-Isopropyl-1H-indol-5-amine

A suspension of 2-isopropyl-5-nitro-1H-indole (100 mg, 0.49 mmol) and Raney Nickel (10 mg) in MeOH (10 mL) was hydrogenated under hydrogen (1 atm) at the room temperature overnight. The catalyst was filtered off through a celite pad and the filtrate was evaporated under vacuum to give a residue, which was purified by column (petroleum ether/ethyl acetate=5/1) to give 2-isopropyl-1H-indol-5-amine (35 mg, 41%). ¹H NMR (400 MHz, CDCl₃) δ 7.69 (br s, 1H), 7.10 (d, J=8.4 Hz, 1H), 6.86 (d, J=2.4 Hz, 1H), 6.58 (dd, J=2.4, 8.8 Hz, 1H), 6.07 (t, J=1.2 Hz, 1H), 3.55 (br s, 2H), 3.06-2.99 (m, 1H), 1.33 (d, J=7.2 Hz, 6H); MS (ESI) m/e (M+H⁺) 175.4.

### Example 45

### 1-(Benzo[d][1,3]dioxol-5-yl)-N-(2-(1-hydroxy-2-methylpropan-2-yl)-1H-indol-5-yl)cyclopropanecarboxamide

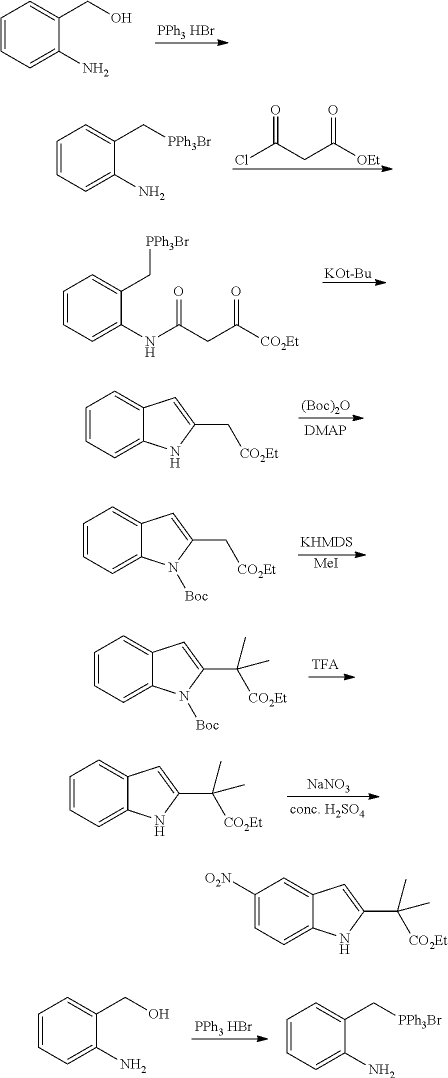

Triphenyl(2-aminobenzyl)phosphonium bromide

2-Aminobenzyl alcohol (60.0 g, 0.487 mol) was dissolved in acetonitrile (2.5 L) and brought to reflux. Triphenylphosphine hydrobromide (167 g, 0.487 mol) was added and the mixture was heated at reflux for 3 h. The reaction mixture was concentrated to approximately 500 mL and left at room temperature for 1 h. The precipitate was filtered and washed with cold acetonitrile followed by hexane. The solid was dried overnight at 40° C. under vacuum to give triphenyl(2-aminobenzyl)phosphonium bromide (193 g, 88%).

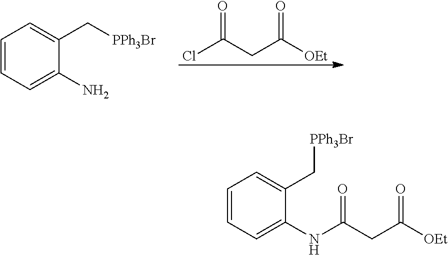

### Triphenyl((ethyl(2-carbamoyl)acetate)-2-benzyl)phosphonium bromide

To a suspension of triphenyl(2-aminobenzyl)phosphonium bromide (190 g, 0.43 mol) in anhydrous dichloromethane (1 L) was added ethyl malonyl chloride (55 ml, 0.43 mol). The reaction was stirred for 3 h at room temperature. The mixture was evaporated to dryness before ethanol (400 mL) was added. The mixture was heated at reflux until a clear solution was obtained. The solution was left at room temperature for 3 h. The precipitate was filtered, washed with cold ethanol followed by hexane and dried. A second crop was obtained from the mother liquor in the same way. In order to remove residual ethanol both crops were combined and dissolved in dichloromethane (approximately 700 mL) under heating and evaporated. The solid was dried overnight at 50° C. under vacuum to give triphenyl((ethyl(2-carbamoyl)acetate)-2-benzyl)-phosphonium bromide (139 g, 58%).

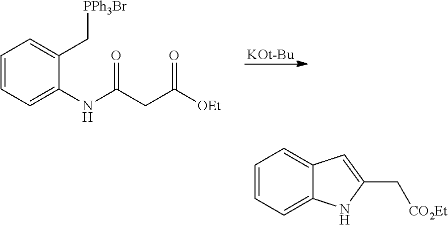

### Ethyl 2-(1H-indol-2-yl)acetate

Triphenyl((ethyl(2-carbamoyl)acetate)-2-benzyl)phosphonium bromide (32.2 g, 57.3 mmol) was added to anhydrous toluene (150 mL) and the mixture was heated at reflux. Fresh potassium tert-butoxide (7.08 g, 63.1 mmol) was added in portions over 15 minutes. Reflux was continued for another 30 minutes. The mixture was filtered hot through a plug of celite and evaporated under reduced pressure. The residue was purified by column chromatography on silica gel (0-30% ethyl acetate in hexane over 45 min) to give ethyl 2-(1H-indol-2-yl)acetate (9.12 g, 78%).

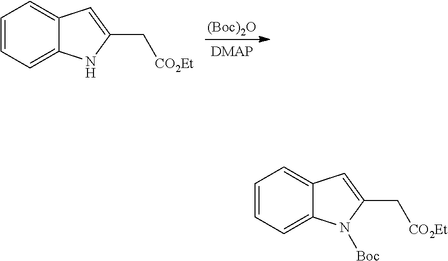 [SMILES: "(Boc)2O" ; CCOC(=O)Cc1cc2ccccc2[nH]1 ; CCOC(=O)Cc1cc2ccccc2n1C(=O)OC(C)(C)C]

### tert-Butyl 2-((ethoxycarbonyl)methyl)-1H-indole-1-carboxylate

To a solution of ethyl 2-(1H-indol-2-yl)acetate (14.7 g, 72.2 mmol) in dichloromethane (150 mL) was added 4-dimethylaminopyridine (8.83 g, 72.2 mmol) and di-tert-butyl carbonate (23.7 g, 108 mmol) in portions. After stirring for 2 h at room temperature, the mixture was diluted with dichloromethane, washed with water, dried over magnesium sulfate and purified by silica gel chromatography (0 to 20% EtOAc in hexane) to give tert-butyl 2-((ethoxycarbonyl)methyl)-1H-indole-1-carboxylate (20.0 g, 91%).

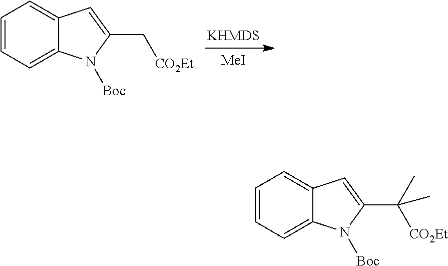 [SMILES: "MeI" ; CCOC(=O)Cc1cc2ccccc2n1C(=O)OC(C)(C)C ; CCOC(=O)C(C)(C)c1cc2ccccc2n1C(=O)OC(C)(C)C]

### tert-Butyl 2-(2-(ethoxycarbonyl)propan-2-yl)-1H-indole-1-carboxylate

tert-Butyl 2-((ethoxycarbonyl)methyl)-1H-indole-1-carboxylate (16.7 g, 54.9 mmol) was added to anhydrous THF (100 mL) and cooled to −78° C. A 0.5M solution of potassium hexamethyldisilazane (165 mL, 82 mmol) was added slowly such that the internal temperature stayed below −60° C. Stirring was continued for 30 minutes at −78° C. To this mixture, methyl iodide (5.64 mL, 91 mmol) was added. The mixture was stirred for 30 min at room temperature and then cooled to −78° C. A 0.5M solution of potassium hexamethyldisilazane (210 mL, 104 mmol) was added slowly and the mixture was stirred for another 30 minutes at −78° C. More methyl iodide (8.6 mL, 137 mmol) was added and the mixture was stirred for 1.5 h at room temperature. The reaction was quenched with sat. aq. ammonium chloride and partitioned between water and dichloromethane. The aqueous phase was extracted with dichloromethane and the combined organic phases were dried over magnesium sulfate and evaporated under reduced pressure. The residue was purified by column chromatography on silica gel (0 to 20% ethylacetate in hexane) to give tert-butyl 2-(2-(ethoxycarbonyl)propan-2-yl)-1H-indole-1-carboxylate (17.1 g, 94%).

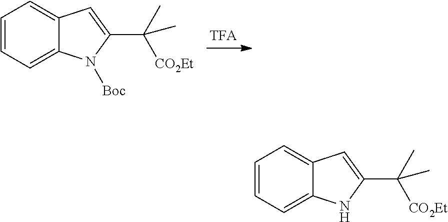 [SMILES: CCOC(=O)C(C)(C)c1cc2ccccc2n1C(=O)OC(C)(C)C ; CCOC(=O)C(C)(C)c1cc2ccccc2[nH]1]

### Ethyl 2-(1H-indol-2-yl)-2-methylpropanoate

tert-Butyl 2-(2-(ethoxycarbonyl)propan-2-yl)-1H-indole-1-carboxylate (22.9 g, 69.1 mmol) was dissolved in dichloromethane (200 mL) before TFA (70 mL) was added. The mixture was stirred for 5 h at room temperature. The mixture was evaporated to dryness, taken up in dichloromethane and washed with saturated sodium bicarbonate solution, water, and brine. The product was purified by column chromatography on silica gel (0-20% EtOAc in hexane) to give ethyl 2-(1H-indol-2-yl)-2-methylpropanoate (12.5 g, 78%).

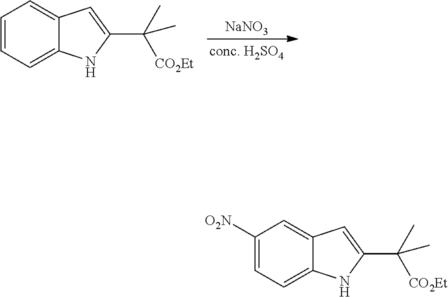

### Ethyl 2-methyl-2-(5-nitro-1H-indol-2-yl)propanoate

Ethyl 2-(1H-indol-2-yl)-2-methylpropanoate (1.0 g, 4.3 mmol) was dissolved in concentrated sulfuric acid (6 mL) and cooled to −10° C. (salt/ice-mixture). A solution of sodium nitrate (370 mg, 4.33 mmol) in concentrated sulfuric acid (3 mL) was added dropwise over 30 min. Stirring was continued for another 30 min at −10° C. The mixture was poured into ice and the product was extracted with dichloromethane. The combined organic phases were washed with a small amount of sat. aq. sodium bicarbonate. The product was purified by column chromatography on silica gel (5-30% EtOAc in hexane) to give ethyl 2-methyl-2-(5-nitro-1H-indol-2-yl)propanoate (0.68 g, 57%).

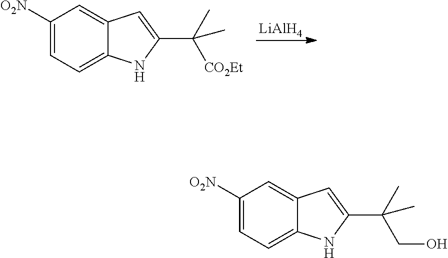

### 2-Methyl-2-(5-nitro-1H-indol-2-yl)propan-1-01

To a cooled solution of LiAlH₄ (1.0 M in THF, 1.1 mL, 1.1 mmol) in THF (5 mL) at 0° C. was added a solution of ethyl 2-methyl-2-(5-nitro-1H-indol-2-yl)propanoate (0.20 g, 0.72 mmol) in THF (3.4 mL) dropwise. After addition, the mixture was allowed to warm up to room temperature and was stirred for 3 h. The mixture was cooled to 0° C. before water (2 mL) was slowly added followed by careful addition of 15% NaOH (2 mL) and water (4 mL). The mixture was stirred at room temperature for 0.5 h and was filtered through a short plug of celite using ethyl acetate. The organic layer was separated from the aqueous layer, dried over Na₂SO₄, filtered and evaporated under reduced pressure. The residue was purified by column chromatography on silica gel (ethyl acetate/hexane=1/1) to give 2-methyl-2-(5-nitro-1H-indol-2-yl)propan-1-ol (0.098 g, 58%).

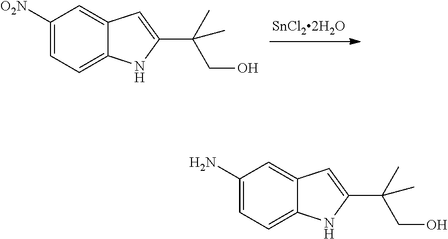 [SMILES: CC(C)(CO)c1cc2cc([N+](=O)[O-])ccc2[nH]1 ; CC(C)(CO)c1cc2cc(N)ccc2[nH]1]

### 2-(5-Amino-1H-indol-2-yl)-2-methylpropan-1-ol

To a solution of 2-methyl-2-(5-nitro-1H-indol-2-yl)propan-1-ol (0.094 g, 0.40 mmol) in ethanol (4 mL) was added tin chloride dihydrate (0.451 g, 2.0 mmol). The mixture was heated in the microwave at 120° C. for 1 h. The mixture was diluted with ethyl acetate and water before being quenched with saturated aqueous NaHCO₃. The reaction mixture was filtered through a plug of celite using ethyl acetate. The organic layer was separated from the aqueous layer, dried over Na₂SO₄, filtered and evaporated under reduced pressure to give 2-(5-amino-1H-indol-2-yl)-2-methylpropan-1-ol (0.080 g, 98%).

### Example 46

### 2-(Pyridin-2-yl)-1H-indol-5-amine

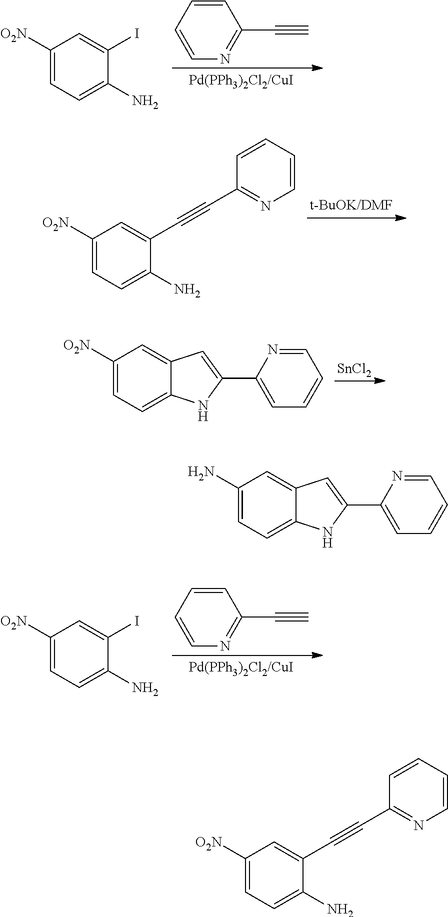

### 4-Nitro-2-(pyridin-2-ylethynyl)aniline

To the solution of 2-iodo-4-nitroaniline (3.0 g, 11 mmol) in DMF (60 mL) and Et₃N (60 mL) was added 2-ethynylpyridine (3.0 g, 45 mmol), Pd(PPh₃)2Cl₂ (600 mg) and CuI (200 mg) under N₂. The reaction mixture was stirred at 60° C. for 12 h. The mixture was diluted with water and extracted with dichloromethane (3×100 mL). The combined organic layers were washed with brine, dried over anhydrous Na₂SO₄ and concentrated in vacuum. The residue was purified by chromatography on silica gel (5-10% ethyl acetate/petroleum ether) to afford 4-nitro-2-(pyridin-2-ylethynyl)aniline (1.5 g, 60%). ¹H NMR (300 MHz, CDCl₃) δ 8.60 (s, 1H), 8.13 (d, J=2.1 Hz, 1H), 7.98 (d, J=1.8, 6.9 Hz, 1H), 7.87-7.80 (m, 2H), 7.42-7.39 (m, 1H), 7.05 (brs, 2H), 6.80 (d, J=6.9 Hz, 1H).

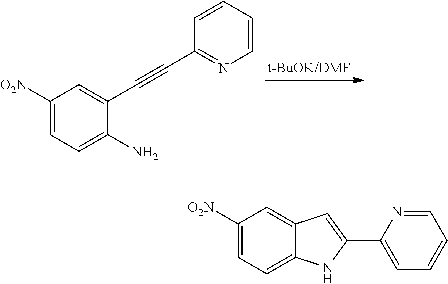 [SMILES: Nc1ccc([N+](=O)[O-])cc1C#Cc1ccccn1 ; O=[N+]([O-])c1ccc2[nH]c(-c3ccccn3)cc2c1]

### 5-Nitro-2-(pyridin-2-yl)-1H-indole

To the solution of 4-nitro-2-(pyridin-2-ylethynyl)aniline (1.5 g, 6.3 mmol) in DMF (50 mL) was added t-BuOK (1.5 g, 13 mmol). The reaction mixture was stirred at 90° C. for 2 h. The mixture was diluted with water and extracted with dichloromethane (3×50 mL). The combined organic layers were washed with brine, dried over anhydrous Na₂SO₄ and concentrated in vacuum. The residue was purified by chromatography on silica gel (5-10% ethyl acetate/petroleum ether) to afford 5-nitro-2-(pyridin-2-yl)-1H-indole (1.0 g, 67% yield). ¹H NMR (300 MHz, d-DMSO) δ 12.40 (s, 1H), 8.66 (d, J=2.1 Hz, 1H), 8.58 (d, J=1.8 Hz, 1H), 8.07-7.91 (m, 3H), 7.59 (d, J=6.6 Hz, 1H), 7.42-7.37 (m, 2H).

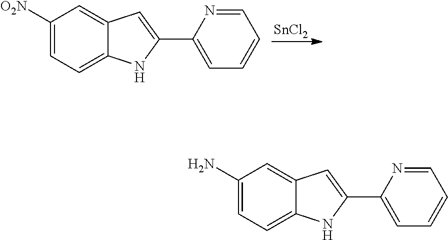

### 2-(Pyridin-2-yl)-1H-indol-5-amine

To a solution of 5-nitro-2-(pyridin-2-yl)-1H-indole (700 mg, 2.9 mmol) in EtOH (20 mL) was added SnCl₂ (2.6 g, 12 mmol). The mixture was heated at reflux for 10 h. Water was added and the mixture was extracted with EtOAc (50 mL×3). The combined organic layers were washed with brine, dried over anhydrous Na₂SO₄ and concentrated in vacuum. The residue was purified by chromatography on silica gel (5-10% ethyl acetate/petroleum ether) to afford 2-(pyridin-2-yl)-1H-indol-5-amine (120 mg, 20%).

NMR (400 MHz, CDCl₃) δ 9.33 (brs, 1H), 8.55 (dd, J=1.2, 3.6 Hz, 1H), 7.76-7.67 (m, 2H), 7.23 (d, J=6.4 Hz, 1H), 7.16-7.12 (m, 1H), 6.94 (d, J=2.0 Hz, 1H), 6.84 (d, J=2.4 Hz, 1H), 6.71-6.69 (dd, J=2.0, 8.4 Hz, 1H).

### Example 47

### 2-(Pyridin-2-yl)-1H-indol-5-amine

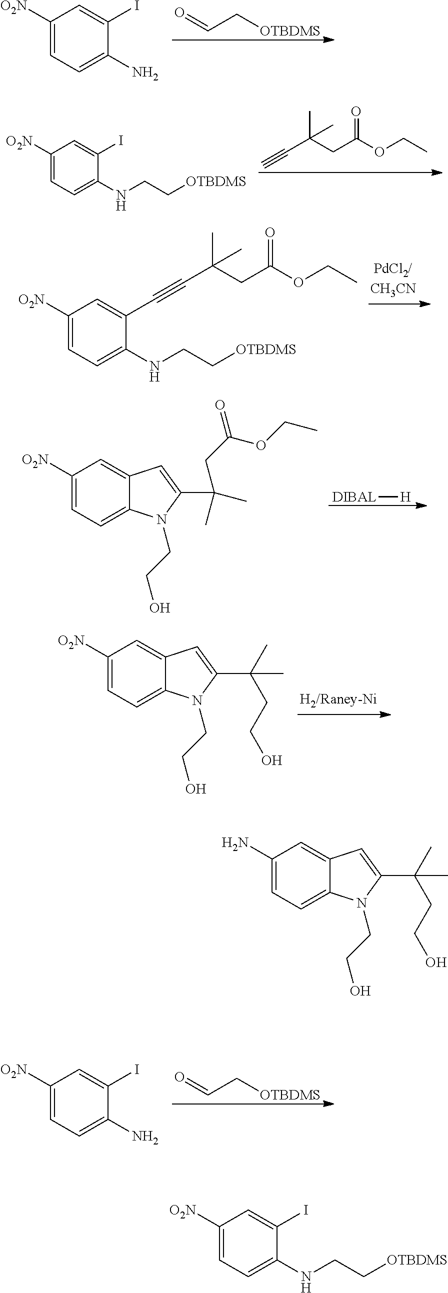

### [2-(tert-Butyl-dimethyl-silanyloxy)-ethyl]-(2-iodo-4-nitro-phenyl)-amine

To a solution of 2-iodo-4-nitroaniline (2.0 g, 7.6 mmol) and 2-(tert-butyldimethylsilyloxy)-acetaldehyde (3.5 g, 75% purity, 15 mmol) in methanol (30 mL) was added TFA (1.5 mL) at 0° C. The reaction mixture was stirred at this temperature for 30 min before NaCNBH₃ (900 mg, 15 mmol) was added in portions. The mixture was stirred for 2 h and was then quenched with water. The resulting mixture was extracted with EtOAc (30 mL×3), the combined organic extracts were dried over anhydrous Na₂SO₄ and evaporated under vacuum, and the residue was purified by chromatography on silica gel (5% ethyl acetate/petroleum) to afford [2-(tert-butyl-dimethyl-silanyloxy)-ethyl]-(2-iodo-4-nitro-phenyl)-amine (800 mg, 25%). ¹H NMR (300 MHz, CDCl₃) δ 8.57 (d, J=2.7 Hz, 1H), 8.12 (dd, J=2.4, 9.0 Hz, 1H), 6.49 (d, J=9.3 Hz, 1H), 5.46 (br s, 1H), 3.89 (t, J=5.4 Hz, 2H), 3.35 (q, J=5.4 Hz, 2H), 0.93 (s, 9H), 0.10 (s, 6H).

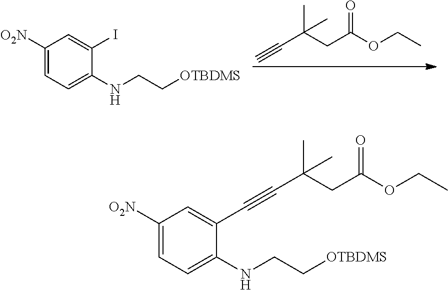 [SMILES: CC(C)(C)[Si](C)(C)OCCNc1ccc([N+](=O)[O-])cc1I ; C#CC(C)(C)CC(=O)OCC ; CCOC(=O)CC(C)(C)C#Cc1cc([N+](=O)[O-])ccc1NCCO[Si](C)(C)C(C)(C)C]

5-{2-[2-(tert-Butyl-dimethyl-silanyloxy)-ethylamino]-5-nitro-phenyl}-3,3-dimethyl-pent-4-ynoic acid ethyl ester

To a solution of [2-(tert-butyl-dimethyl-silanyloxy)-ethyl]-(2-iodo-4-nitro-phenyl)-amine (800 mg, 1.9 mmol) in Et₃N (20 mL) was added Pd(PPh₃)2Cl₂ (300 mg, 0.040 mmol), CuI (76 mg, 0.040 mmol) and 3,3-dimethyl-but-1-yne (880 mg, 5.7 mmol) successively under N₂ protection. The reaction mixture was heated at 80° C. for 6 h and allowed to cool down to room temperature. The resulting mixture was extracted with EtOAc (30 mL×3). The combined organic extracts were dried over anhydrous Na₂SO₄ and evaporated under vacuum to give 5-{2-[2-(tert-butyl-dimethyl-silanyloxy)-ethylamino]-5-nitro-phenyl}-3,3-dimethyl-pent-4-ynoic acid ethyl ester (700 mg, 82%), which was used in the next step without further purification. ¹H NMR (400 MHz, CDCl₃) δ 8.09 (s, 1H), 8.00 (d, J=9.2 Hz, 1H), 6.54 (d, J=9.2 Hz, 1H), 6.45 (brs, 1H), 4.17-4.10 (m, 4H), 3.82 (t, J=5.6 Hz, 2H), 3.43 (q, J=5.6 Hz, 2H), 2.49 (s, 2H), 1.38 (s, 6H), 1.28 (t, J=7.2 Hz, 3H), 0.84 (s, 9H), 0.00 (s, 6H).

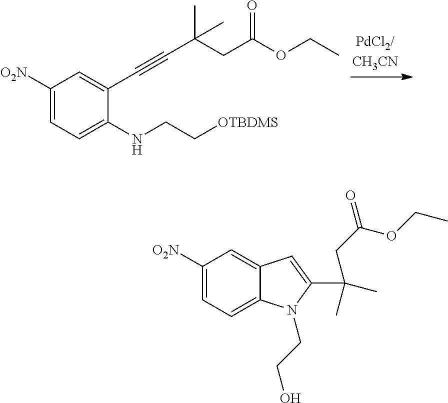 [SMILES: CCOC(=O)CC(C)(C)C#Cc1cc([N+](=O)[O-])ccc1NCCO[Si](C)(C)C(C)(C)C ; CCOC(=O)CC(C)(C)c1cc2cc([N+](=O)[O-])ccc2n1CCO]

### 3-[1-(2-Hydroxy-ethyl)-5-nitro-1H-indol-2-yl]-3-methyl-butyric acid ethyl ester

A solution of 5-{2-[2-(tert-butyl-dimethyl-silanyloxy)-ethylamino]-5-nitro-phenyl}-3,3-dimethyl-pent-4-ynoic acid ethyl ester (600 mg, 1.34 mmol) and PdCl₂(650 mg) in CH₃CN (30 mL) was heated at reflux overnight. The resulting mixture was extracted with EtOAc (30 mL×3). The combined organic extracts were dried over anhydrous Na₂SO₄ and evaporated under vacuum. The residue was dissolved in THF (20 mL) and TBAF (780 mg, 3.0 mmol) was added. The mixture was stirred at room temperature for 1 h, the solvent was removed under vacuum, and the residue was purified by chromatography on silica gel (10% ethyl acetate/petroleum) to afford 3-[1-(2-hydroxy-ethyl)-5-nitro-1H-indol-2-yl]-3-methyl-butyric acid ethyl ester (270 mg, 60%). ¹H NMR (300 MHz, CDCl₃) δ 8.45 (d, J=2.1 Hz, 1H), 8.05 (dd, J=2.1, 9.0 Hz, 1H), 6.36 (d, J=9.0 Hz, 1H), 6.48 (s, 1H), 4.46 (t, J=6.6 Hz, 2H), 4.00-3.91 (m, 4H), 2.76 (s, 2H), 1.61 (s, 6H), 0.99 (t, J=7.2 Hz, 1H), 0.85 (s, 9H), 0.03 (s, 6H).

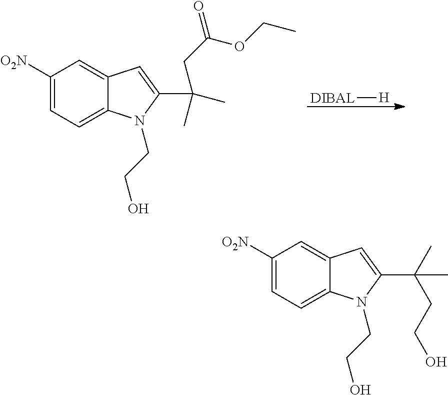

### 3-[1-(2-Hydroxy-ethyl)-5-nitro-1H-indol-2-yl]-3-methyl-butan-1-ol

To a solution of 3-[1-(2-hydroxy-ethyl)-5-nitro-1H-indol-2-yl]-3-methyl-butyric acid ethyl ester (700 mg, 2.1 mmol) in THF (25 mL) was added DIBAL-H (1.0 M, 4.2 mL, 4.2 mmol) at −78° C. The mixture was stirred at room temperature for 1 h. Water (2 mL) was added and the resulting mixture was extracted with EtOAc (15 mL×3). The combined organic layers were dried over anhydrous Na₂SO₄ and evaporated under vacuum. The residue was purified by chromatography on silica gel (15% ethyl acetate/petroleum) to afford 3-[1-(2-hydroxy-ethyl)-5-nitro-1H-indol-2-yl]-3-methyl-butan-1-ol (300 mg, 49%). ¹H NMR (300 MHz, d-DMSO) δ 8.42 (d, J=1.5 Hz, 1H), 7.95 (dd, J=1.2, 8.7 Hz, 1H), 6.36 (d, J=9.3 Hz, 1H), 6.50 (s, 1H), 5.25 (br s, 1H), 4.46-4.42 (m, 4H), 3.69-3.66 (m, 2H), 3.24-3.21 (m, 2H), 1.42 (s, 6H).

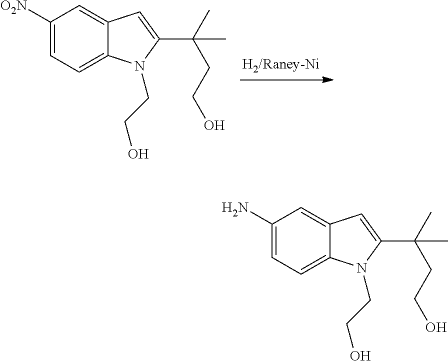 [SMILES: CC(C)(CCO)c1cc2cc([N+](=O)[O-])ccc2n1CCO ; CC(C)(CCO)c1cc2cc(N)ccc2n1CCO]

### 3-[5-Amino-1-(2-hydroxy-ethyl)-1H-indol-2-yl]-3-methyl-butan-1-01

A solution of 3-[1-(2-hydroxy-ethyl)-5-nitro-1H-indol-2-yl]-3-methyl-butan-1-ol (300 mg, 1.03 mmol) and Raney Nickel (200 mg,) in CH₃OH (30 mL) was stirred for 5 h at room temperature under a H₂ atmosphere. The catalyst was filtered through a celite pad and the filtrate was evaporated under vacuum to give a residue, which was purified by preparative TLC to afford 3-[5-amino-1-(2-hydroxy-ethyl)-1H-indol-2-yl]-3-methyl-butan-1-ol (70 mg, 26%). ¹H NMR (300 MHz, CDCl₃) δ 7.07 (d, J=8.7 Hz, 1H), 6.83 (d, J=2.1 Hz, 1H), 6.62 (dd, J=2.1, 8.4 Hz, 1H), 6.15 (s, 1H), 4.47 (t, J=5.4 Hz, 2H), 4.07 (t, J=5.4 Hz, 2H), 3.68 (t, J=5.7 Hz, 2H), 2.16 (t, J=5.7 Hz, 2H), 4.00-3.91 (m, 4H), 2.76 (s, 2H), 1.61 (s, 6H), 1.42 (s, 6H).

### Example 48

### tert-Butyl 2-(5-amino-1H-indol-2-yl)piperidine-1-carboxylate

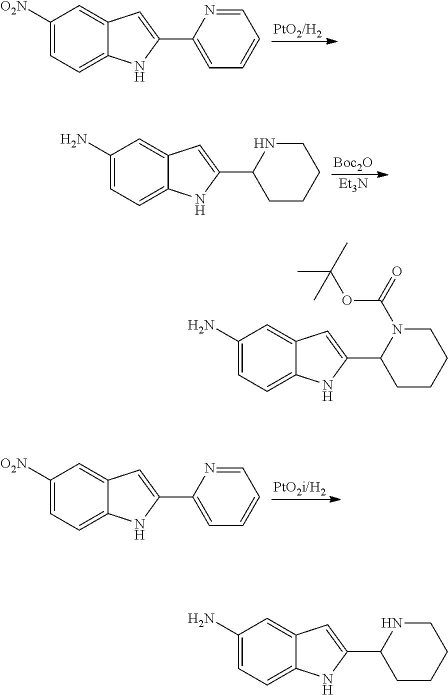

### 2-(Piperidin-2-yl)-1H-indol-5-amine

5-Nitro-2-(pyridin-2-yl)-1H-indole (1.0 g, 4.2 mmol) was added to HCl/MeOH (2 M, 50 mL). The reaction mixture was stirred at room temperature for 1 h and the solvent was evaporated under vacuum. PtO₂ (200 mg) was added to a solution of the residue in MeOH (50 mL) and the reaction mixture was stirred under hydrogen atmosphere (1 atm) at room temperature for 2 h. The catalyst was filtered through a celite pad and the solvent was evaporated under vacuum to afford 2-(piperidin-2-yl)-1H-indol-5-amine (1.0 g), which was directly used in the next step.

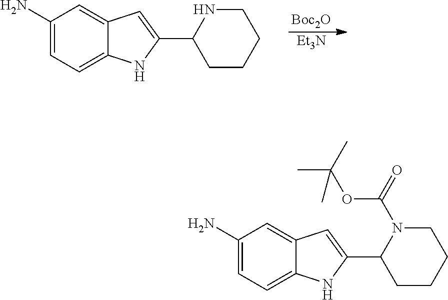 [SMILES: "Boc2O" ; "Et3N" ; Nc1ccc2[nH]c(C3CCCCN3)cc2c1 ; CC(C)(C)OC(=O)N1CCCCC1c1cc2cc(N)ccc2[nH]1]

### tert-Butyl 2-(5-amino-1H-indol-2-yl)piperidine-1-carboxylate

To a solution of 2-(piperidin-2-yl)-1H-indol-5-amine (1.0 g) in Et₃N (25 mL) and THF (25 mL) was added Boc₂O (640 mg, 2.9 mmol). The reaction mixture was stirred at room temperature overnight. The mixture was diluted with water and extracted with dichloromethane (3×25 mL). The combined organic layers were washed with brine, dried over anhydrous Na₂SO₄ and concentrated in vacuum. The residue was purified by chromatography on silica gel (5-10% ethyl acetate/petroleum ether) followed by preparative HPLC to afford tert-butyl 2-(5-amino-1H-indol-2-yl)piperidine-1-carboxylate (15 mg, 1% over 2 steps). ¹H NMR (400 MHz, CDCl₃) δ 8.82 (s, 1H), 7.58 (s, 1H), 7.22 (d, J=8.8 Hz, 1H), 7.02 (d, J=1.6, 8.0 Hz, 1H), 6.42 (s, 1H), 6.25 (s, 1H), 3.91-3.88 (m, 1H), 3.12-3.10 (m, 1H), 2.81-2.76 (m, 1H), 2.06-1.97 (m, 4H), 1.70-1.58 (m, 2H), 1.53 (s, 9H).

### Example 49

### 6-amino-1H-indole-2-carbonitrile

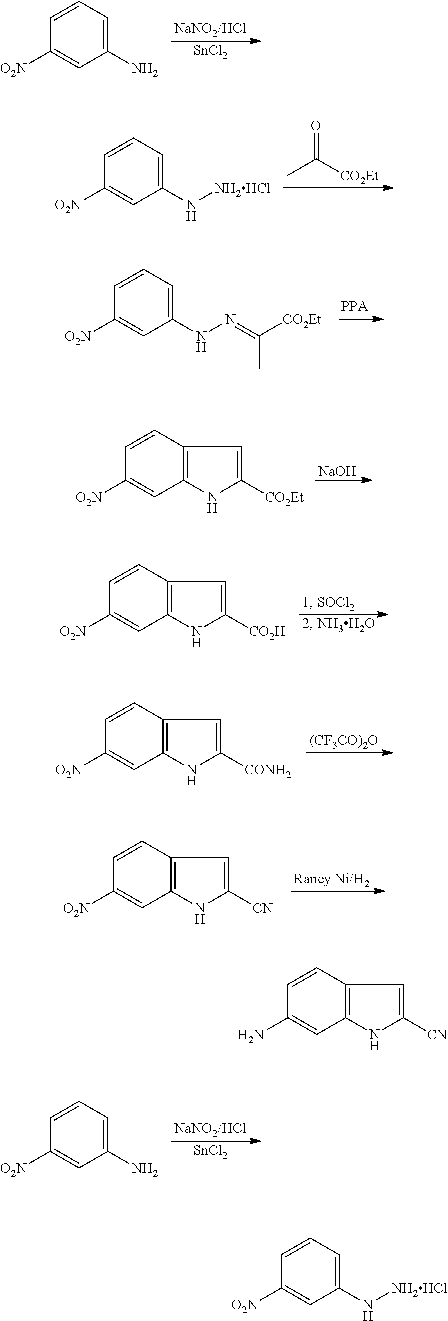

### (3-Nitrophenyl)hydrazine hydrochloride

3-Nitroaniline (28 g, 0.20 mol) was dissolved in a mixture of H₂O (40 mL) and 37% HCl (40 mL). A solution of NaNO₂ (14 g, 0.20 mol) in H₂O (60 mL) was added to the mixture at 0° C., and then a solution of SnCl₂.H₂O (140 g, 0.60 mol) in 37% HCl (100 mL) was added. After stirring at 0° C. for 0.5 h, the insoluble material was isolated by filtration and was washed with water to give (3-nitrophenyl)hydrazine hydrochloride (28 g, 73%).

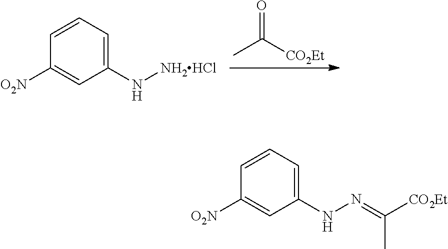

### (E)-Ethyl 2-(2-(3-nitrophenyl)hydrazono)propanoate

(3-Nitrophenyl)hydrazine hydrochloride (30 g, 0.16 mol) and 2-oxo-propionic acid ethyl ester (22 g, 0.19 mol) were dissolved in ethanol (300 mL). The mixture was stirred at room temperature for 4 h before the solvent was evaporated under reduced pressure to give (E)-ethyl 2-(2-(3-nitrophenyl)hydrazono)propanoate, which was used directly in the next step.

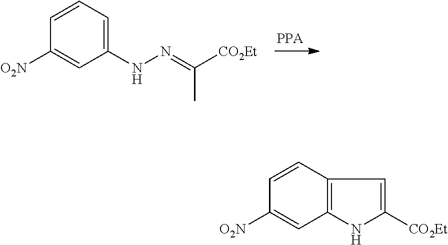 [SMILES: CCOC(=O)/C(C)=N/Nc1cccc([N+](=O)[O-])c1 ; CCOC(=O)c1cc2ccc([N+](=O)[O-])cc2[nH]1]

### Ethyl 4-nitro-1H-indole-2-carboxylate and ethyl 6-nitro-1H-indole-2-carboxylate

(E)-Ethyl 2-(2-(3-nitrophenyl)hydrazono)propanoate was dissolved in toluene (300 mL) and PPA (30 g) was added. The mixture was heated at reflux overnight and then was cooled to room temperature. The solvent was decanted and evaporated to obtain a crude mixture that was taken on to the next step without purification (15 g, 40%).

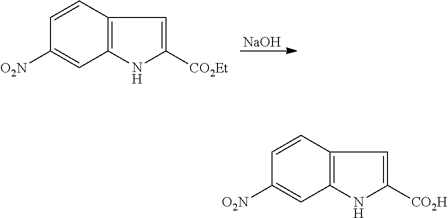

### 4-Nitro-1H-indole-2-carboxylic acid and 6-nitro-1H-indole-2-carboxylic acid

A mixture of ethyl 6-nitro-1H-indole-2-carboxylate (0.5 g) and 10% NaOH (20 mL) was heated at reflux overnight and then was cooled to room temperature. The mixture was extracted with ether and the aqueous phase was acidified with HCl to pH 1-2. The insoluble solid was isolated by filtration to give a crude mixture that was taken on to the next step without purification (0.3 g, 68%).

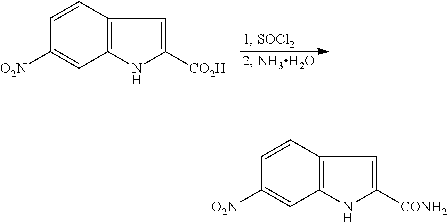 [SMILES: O=C(O)c1cc2ccc([N+](=O)[O-])cc2[nH]1 ; "2, NH3·H2O" ; "1, SOCl2" ; NC(=O)c1cc2ccc([N+](=O)[O-])cc2[nH]1]

### 4-Nitro-1H-indole-2-carboxamide and 6-nitro-1H-indole-2-carboxamide

A mixture of 6-nitro-1H-indole-2-carboxylic acid (12 g, 58 mmol) and SOCl₂ (50 mL, 64 mmol) in benzene (150 mL) was heated at reflux for 2 h. The benzene and excess SOCl₂ was removed under reduced pressure. The residue was dissolved in anhydrous CH₂Cl₂ (250 mL) and NH₃.H₂O (22 g, 0.32 mol) was added dropwise at 0° C. The mixture was stirred at room temperature for 1 h. The insoluble solid was isolated by filtration to obtain crude mixture (9.0 g, 68%), which was used directly in the next step.

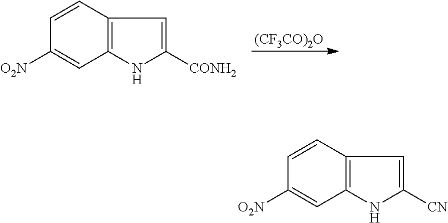

### 4-Nitro-1H-indole-2-carbonitrile and 6-nitro-1H-indole-2-carbonitrile

6-Nitro-1H-indole-2-carboxamide (5.0 g, 24 mmol) was dissolved in CH₂Cl₂ (200 mL). Et₃N (24 g, 0.24 mol) and (CF₃CO)2O (51 g, 0.24 mol) were added dropwise to the mixture at room temperature. The mixture was continued to stir for 1 h and was then poured into water (100 mL). The organic layer was separated and the aqueous layer was extracted with EtOAc (100 mL three times). The combined organic layers were dried over Na₂SO₄, filtered and concentrated under reduced pressure to obtain crude product which was purified by column chromatography on silica gel to give a impure sample of 4-nitro-1H-indole-2-carbonitrile (2.5 g, 55%).

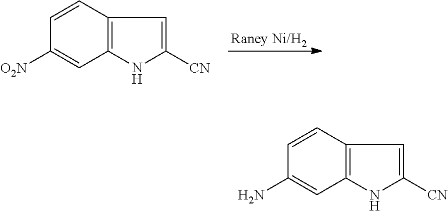 [SMILES: N#Cc1cc2ccc([N+](=O)[O-])cc2[nH]1 ; "Raney Ni/H2" ; N#Cc1cc2ccc(N)cc2[nH]1]

### 6-Amino-1H-indole-2-carbonitrile

A mixture of 6-nitro-1H-indole-2-carbonitrile (2.5 g, 13 mmol) and Raney Nickel (500 mg) in EtOH (50 mL) was stirred at room temperature under H₂ (1 atm) for 1 h. Raney Nickel was removed via filtration and the filtrate was evaporated under reduced pressure to give a residue, which was purified by column chromatography on silica get to give 6-amino-1H-indole-2-carbonitrile (1.0 g, 49%). ¹H NMR (DMSO-d₆) δ 12.75 (br s, 1H), 7.82 (d, J=8 Hz, 1H), 7.57 (s, 1H), 7.42 (s, 1H), 7.15 (d, J=8 Hz, 1H); MS (ESI) m/e (M+H⁺) 158.2.

### Example 50

### 6-Amino-1H-indole-3-carbonitrile

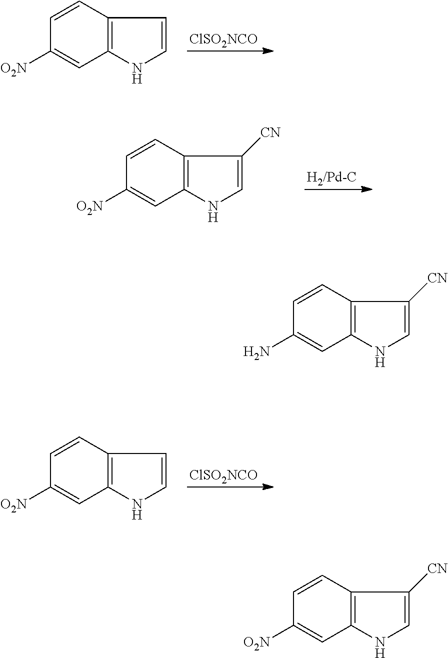

### 6-Nitro-1H-indole-3-carbonitrile

To a solution of 6-nitroindole (4.9 g 30 mmol) in DMF (24 mL) and CH₃CN (240 mL) was added dropwise a solution of ClSO₂NCO (5.0 mL) in CH₃CN (39 mL) at 0° C. After addition, the reaction was allowed to warm to room temperature and was stirred for 2 h. The mixture was then poured into ice-water and basified with sat. NaHCO₃ solution to pH 7-8. The mixture was extracted with ethyl acetate. The organics were washed with brine, dried over Na₂SO₄ and concentrated to give 6-nitro-1H-indole-3-carbonitrile (4.6 g, 82%).

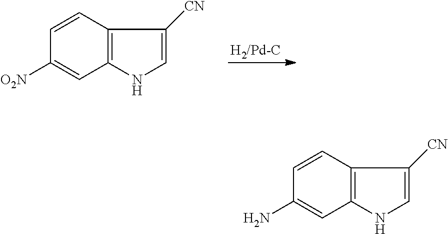 [SMILES: N#Cc1c[nH]c2cc([N+](=O)[O-])ccc12 ; "H2/Pd-C" ; N#Cc1c[nH]c2cc(N)ccc12]

### 6-Amino-1H-indole-3-carbonitrile

A suspension of 6-nitro-1H-indole-3-carbonitrile (4.6 g, 25 mmol) and 10% Pd—C (0.46 g) in EtOH (50 mL) was stirred under H₂ (1 atm) at room temperature overnight. After filtration, the filtrate was concentrated and the residue was purified by column chromatography on silica gel (petroleum ether/ethyl acetate=3/1) to give 6-amino-1H-indole-3-carbonitrile (1.0 g, 98%) as a pink solid. ¹H NMR (DMSO-d₆) δ 11.51 (s, 1H), 7.84 (d, J=2.4 Hz, 1H), 7.22 (d, J=8.4 Hz, 1H), 6.62 (s, 1H), 6.56 (d, J=8.4 Hz, 1H), 5.0 (s, 2H); MS (ESI) m/e (M+H⁺) 157.1.

### Example 51

### 3 2-tert-Butyl-1H-indol-6-amine

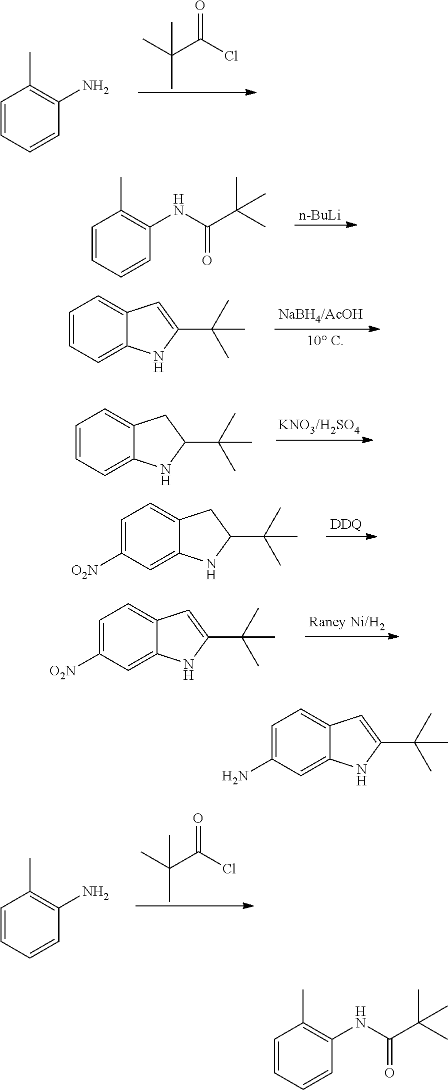

### N-o-Tolylpivalamide

To a solution of o-tolylamine (21 g, 0.20 mol) and Et₃N (22 g, 022 mol) in CH₂Cl₂ was added 2,2-dimethyl-propionyl chloride (25 g, 0.21 mol) at 10° C. After addition, the mixture was stirred overnight at room temperature. The mixture was washed with aq. HCl (5%, 80 mL), saturated aq. NaHCO₃ and brine. The organic layer was dried over Na₂SO₄ and concentrated under vacuum to give N-o-tolylpivalamide (35 g, 91%). ¹H NMR (300 MHz, CDCl₃) δ 7.88 (d, J=7.2 Hz, 1H), 7.15-7.25 (m, 2H), 7.05 (t, J=7.2 Hz, 1H), 2.26 (s, 3H), 1.34 (s, 9H).

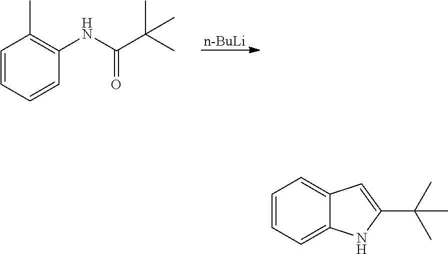

### 2-tert-Butyl-1H-indole

To a solution of N-o-tolylpivalamide (30.0 g, 159 mmol) in dry THF (100 mL) was added dropwise n-BuLi (2.5 M in hexane, 190 mL) at 15° C. After addition, the mixture was stirred overnight at 15° C. The mixture was cooled in an ice-water bath and treated with saturated NH₄Cl. The organic layer was separated and the aqueous layer was extracted with ethyl acetate. The combined organic layers were dried over anhydrous Na₂SO₄, filtered, and concentrated in vacuum. The residue was purified by column chromatography on silica gel to give 2-tert-butyl-1H-indole (24 g, 88%). ¹H NMR (300 MHz, CDCl₃) δ 7.99 (br. s, 1H), 7.54 (d, J=7.2 Hz, 1H), 7.05 (d, J=7.8 Hz, 1H), 7.06-7.13 (m, 2H), 6.26 (s, 1H), 1.39 (s, 9H).

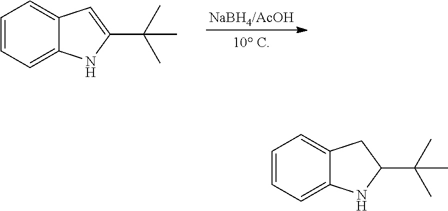

### 2-tert-Butylindoline

To a solution of 2-tert-butyl-1H-indole (10 g, 48 mmol) in AcOH (40 mL) was added NaBH₄ at 10° C. The mixture was stirred for 20 minutes at 10° C. before being treated dropwise with H₂O under ice cooling. The mixture was extracted with ethyl acetate. The combined organic layers were dried over anhydrous Na₂SO₄, filtered, and concentrated under vacuum to give 2-tert-butylindoline (9.8 g), which was used directly in the next step.

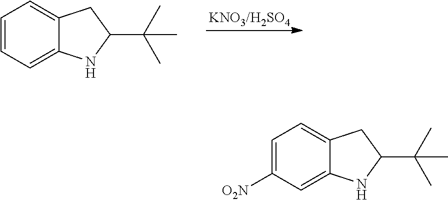 [SMILES: CC(C)(C)C1Cc2ccccc2N1 ; CC(C)(C)C1Cc2ccc([N+](=O)[O-])cc2N1]

### 2-tert-butyl-6-nitroindoline and 2-tert-butyl-5-nitro-1H-indole

To a solution of 2-tert-butylindoline (9.7 g) in H₂SO₄ (98%, 80 mL) was slowly added KNO₃ (5.6 g, 56 mmol) at 0° C. After addition, the reaction mixture was stirred at room temperature for 1 h. The mixture was carefully poured into cracked ice, basified with Na₂CO₃ to pH 8 and extracted with ethyl acetate. The combined extracts were washed with brine, dried over anhydrous Na₂SO₄ and concentrated under vacuum. The residue was purified by column chromatography to give 2-tert-butyl-6-nitroindoline (4.0 g, 31% over two steps). ¹H NMR (300 MHz, CDCl₃) δ 7.52 (dd, J=1.8, 8.1 Hz, 1H), 7.30 (s, 1H), 7.08 (d, J=7.8 Hz, 1H), 3.76 (t, J=9.6 Hz, 1H), 2.98-3.07 (m, 1H), 2.82-2.91 (m, 1H), 0.91 (s, 9H).

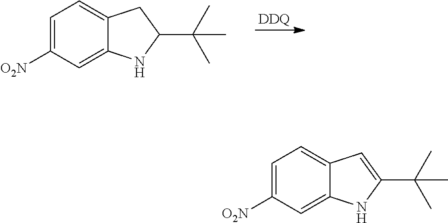

### 2-tert-Butyl-6-nitro-1H-indole

To a solution of 2-tert-butyl-6-nitroindoline (2.0 g, 9.1 mmol) in 1,4-dioxane (20 mL) was added DDQ (6.9 g, 30 mmol) at room temperature. The mixture was heated at reflux for 2.5 h before being filtered and concentrated under vacuum. The residue was purified by column chromatography to give 2-tert-butyl-6-nitro-1H-indole (1.6 g, 80%). ¹H NMR (300 MHz, CDCl₃) δ 8.30 (br. s, 1H), 8.29 (s, 1H), 8.00 (dd, J=2.1, 8.7 Hz, 1H), 7.53 (d, J=9.3 Hz, 1H), 6.38 (s, 1H), 1.43 (s, 9H).

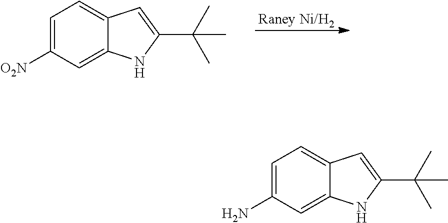

### 2-tert-Butyl-1H-indol-6-amine

To a solution of 2-tert-butyl-6-nitro-1H-indole (1.3 g, 6.0 mmol) in MeOH (10 mL) was added Raney Nickel (0.2 g). The mixture was hydrogenated under 1 atm of hydrogen at room temperature for 3 h. The reaction mixture was filtered and the filtrate was concentrated. The residue was washed with petroleum ether to give 2-tert-butyl-1H-indol-6-amine (1.0 g, 89%). ¹H NMR (300 MHz, DMSO-d₆) δ 10.19 (s, 1H), 6.99 (d, J=8.1 Hz, 1H), 6.46 (s, 1H), 6.25 (dd, J=1.8, 8.1 Hz, 1H), 5.79 (d, J=1.8 Hz, 1H), 4.52 (s, 2H), 1.24 (s, 9H); MS (ESI) m/e (M+H⁺) 189.1.

### Example 52

### 3-tert-Butyl-1H-indol-6-amine

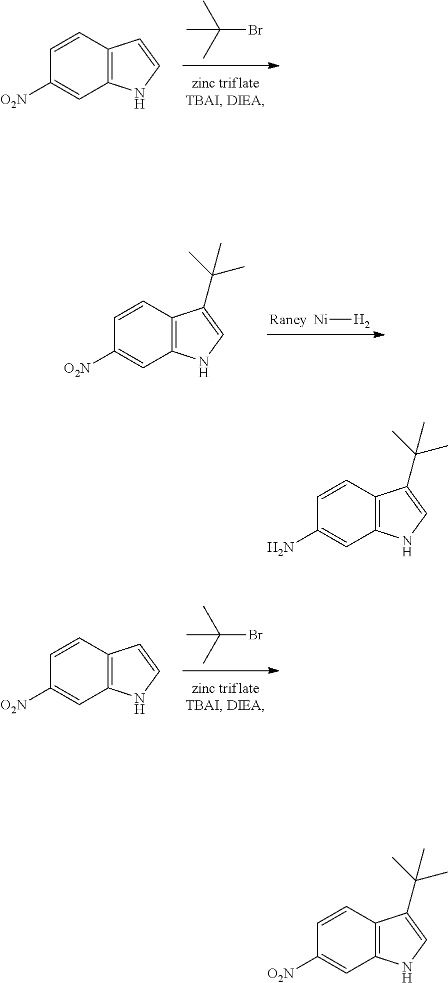

### 3-tert-Butyl-6-nitro-1H-indole

To a mixture of 6-nitroindole (1.0 g, 6.2 mmol), zinc triflate (2.1 g, 5.7 mmol), and TBAI (1.7 g, 5.2 mmol) in anhydrous toluene (11 mL) was added DIEA (1.5 g, 11 mmol) at room temperature under nitrogen. The reaction mixture was stirred for 10 min at 120° C., followed by the addition of t-butyl bromide (0.71 g, 5.2 mmol). The resulting mixture was stirred for 45 min at 120° C. The solid was filtered off and the filtrate was concentrated to dryness. The residue was purified by column chromatography on silica gel (petroleum ether/ethyl acetate=20:1) to give 3-tert-butyl-6-nitro-1H-indole (0.25 g, 19%) as a yellow solid. ¹H-NMR (CDCl₃) δ 8.32 (d, J=2.1 Hz, 1H), 8.00 (dd, J=2.1, 14.4 Hz, 1H), 7.85 (d, J=8.7 Hz, 1H), 7.25 (s, 1H), 1.46 (s, 9H).

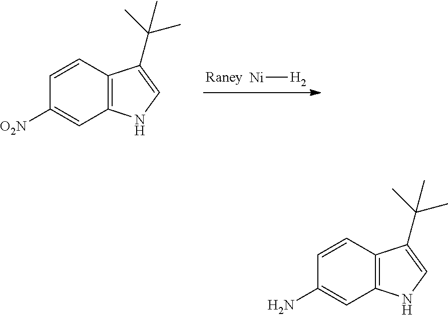

### 3-tert-Butyl-1H-indol-6-amine

A suspension of 3-tert-butyl-6-nitro-1H-indole (3.0 g, 14 mmol) and Raney Nickel (0.5 g) was hydrogenated under H₂ (1 atm) at room temperature for 3 h. The catalyst was filtered off and the filtrate was concentrated to dryness. The residue was purified by column on silica gel (petroleum ether/ethyl acetate=4:1) to give 3-tert-butyl-1H-indol-6-amine (2.0 g, 77%) as a gray solid. ¹HNMR (CDCl₃) δ 7.58 (m, 2H), 6.73 (d, J=1.2 Hz, 1H), 6.66 (s, 1H), 6.57 (dd, J=0.8, 8.6 Hz, 1H), 3.60 (br, 2H), 1.42 (s, 9H).

### Example 53

### 5-(Trifluoromethyl)-1H-indol-6-amine

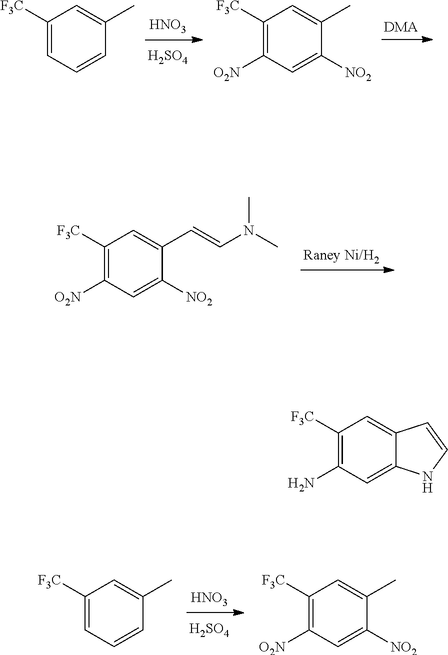

### 1-Methyl-2,4-dinitro-5-(trifluoromethyl)benzene

To a mixture of HNO₃ (98%, 30 mL) and H₂SO₄ (98%, 30 mL) was added dropwise 1-methyl-3-trifluoromethyl-benzene (10 g, 63 mmol) at 0° C. After addition, the mixture was stirred at rt for 30 min and was then poured into ice-water. The precipitate was filtered and washed with water to give 1-methyl-2,4-dinitro-5-trifluoromethyl-benzene (2.0 g, 13%).

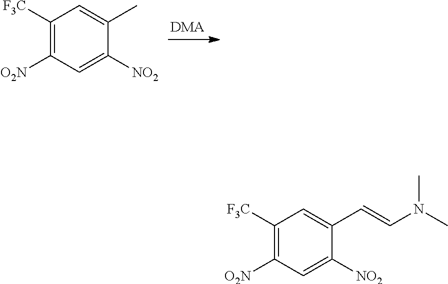

### (E)-2-(2,4-Dinitro-5-(trifluoromethyl)phenyl)-N,N-dimethylethenamine

A mixture of 1-methyl-2,4-dinitro-5-trifluoromethyl-benzene (2.0 g, 8.0 mmol) and DMA (1.0 g, 8.2 mmol) in DMF (20 mL) was stirred at 100° C. for 30 min. The mixture was poured into ice-water and stirred for 1 h. The precipitate was filtered and washed with water to give (E)-2-(2,4-dinitro-5-(trifluoromethyl)phenyl)-N,N-dimethylethenamine (2.1 g, 86%).

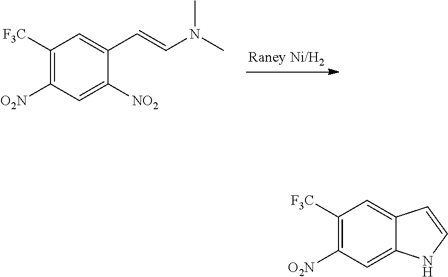

### 5-(Trifluoromethyl)-1H-indol-6-amine

A suspension of (E)-2-(2,4-dinitro-5-(trifluoromethyl)phenyl)-N,N-dimethylethenamine (2.1 g, 6.9 mmol) and Raney Nickel (1 g) in ethanol (80 mL) was stirred under H₂ (1 atm) at room temperature for 5 h. The catalyst was filtered off and the filtrate was concentrated to dryness. The residue was purified by column on silica gel to give 5-(trifluoromethyl)-1H-indol-6-amine (200 mg, 14%). ¹H NMR (DMSO-d₆) δ 10.79 (br s, 1H), 7.55 (s, 1H), 7.12 (s, 1H), 6.78 (s, 1H), 6.27 (s, 1H), 4.92 (s, 2H); MS (ESI) m/e (M+H⁺): 200.8.

### Example 54

### 5-Ethyl-1H-indol-6-amine

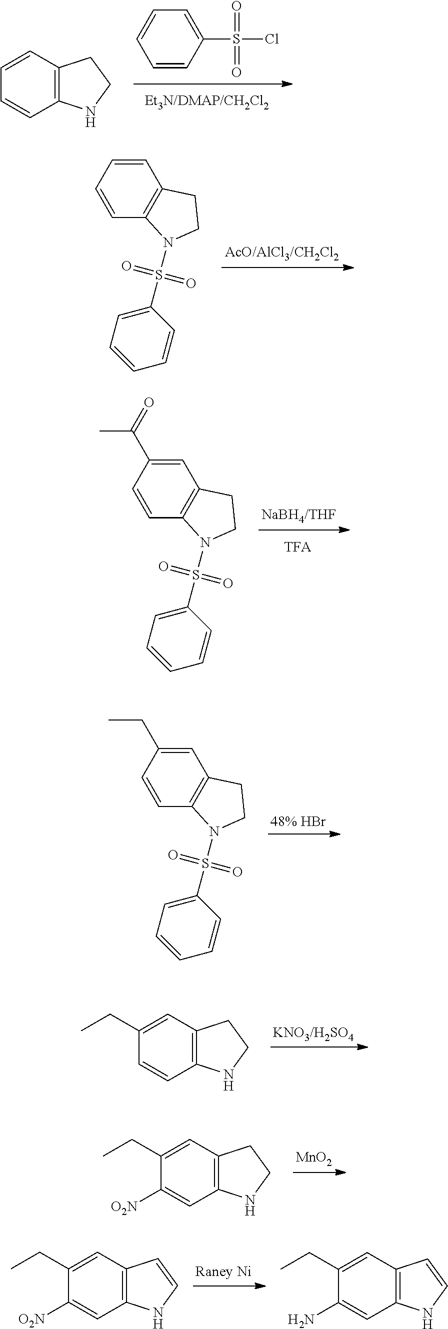

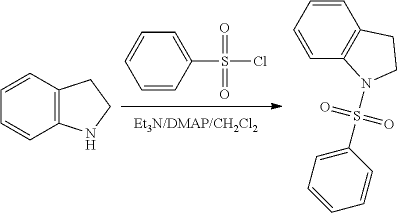

### 1-(Phenylsulfonyl)indoline

To a mixture of DMAP (1.5 g), benzenesulfonyl chloride (24.0 g, 136 mmol) and indoline (14.7 g, 124 mmol) in CH₂Cl₂ (200 mL) was added dropwise Et₃N (19.0 g, 186 mmol) at 0° C. The mixture was stirred at room temperature overnight. The organic layer was washed with water (2×), dried over Na₂SO₄ and concentrated to dryness under reduced pressure to obtain 1-(phenylsulfonyl)indoline (30.9 g, 96%).

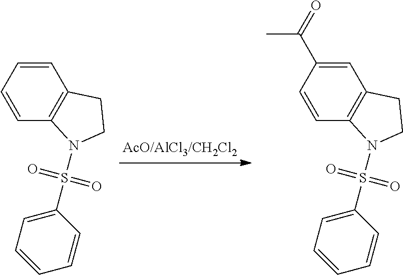 [SMILES: O=S(=O)(c1ccccc1)N1CCc2ccccc21 ; CC(=O)c1ccc2c(c1)CCN2S(=O)(=O)c1ccccc1]

### 1-(1-(Phenylsulfonyl)indolin-5-yl)ethanone

To a suspension of AlCl₃ (144 g, 1.08 mol) in CH₂Cl₂ (1070 mL) was added acetic anhydride (54 mL). The mixture was stirred for 15 minutes before a solution of 1-(phenylsulfonyl)indoline (46.9 g, 0.180 mol) in CH₂Cl₂ (1070 mL) was added dropwise. The mixture was stirred for 5 h and was quenched by the slow addition of crushed ice. The organic layer was separated and the aqueous layer was extracted with CH₂Cl₂. The combined organics were washed with saturated aqueous NaHCO₃ and brine, dried over Na₂SO₄, and concentrated under vacuum to obtain 1-(1-(phenylsulfonyl)indolin-5-yl)ethanone (42.6 g).

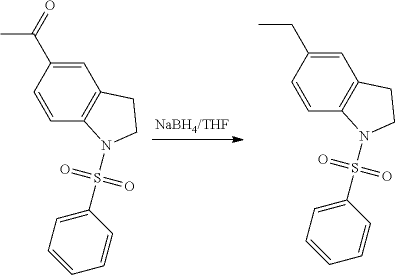 [SMILES: CC(=O)c1ccc2c(c1)CCN2S(=O)(=O)c1ccccc1 ; CCc1ccc2c(c1)CCN2S(=O)(=O)c1ccccc1]

### 5-Ethyl-1-(phenylsulfonyl)indoline

To TFA (1600 mL) at 0° C. was added sodium borohydride (64.0 g, 1.69 mol) over 1 h. To this mixture was added dropwise a solution of 1-(1-(phenylsulfonyl)indolin-5-yl)ethanone (40.0 g, 0.133 mol) in TFA (700 mL) over 1 h. The mixture was then stirred overnight at 25° C. After dilution with H₂O (1600 mL), the mixture was made basic by the addition of sodium hydroxide pellets at 0° C. The organic layer was separated and the aqueous layer was extracted with CH₂Cl₂. The combined organic layers were washed with brine, dried over Na₂SO₄ and concentrated under reduced pressure. The residue was purified by silica column to give 5-ethyl-1-(phenylsulfonyl)indoline (16.2 g, 47% over two steps).

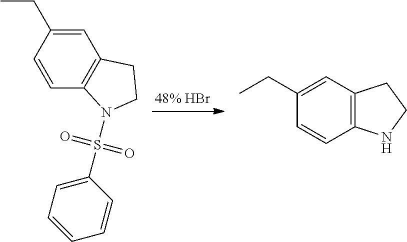 [SMILES: CCc1ccc2c(c1)CCN2S(=O)(=O)c1ccccc1 ; CCc1ccc2c(c1)CCN2]

### 5-Ethylindoline

A mixture of 5-ethyl-1-(phenylsulfonyl)indoline (15 g, 0.050 mol) in HBr (48%, 162 mL) was heated at reflux for 6 h. The mixture was basified with sat. NaOH to pH 9 and then it was extracted with ethyl acetate. The organic layer was washed with brine, dried over Na₂SO₄, and concentrated under reduced pressure. The residue was purified by silica column to give 5-ethylindoline (2.5 g, 32%).

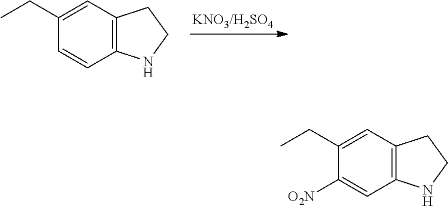

### 5-Ethyl-6-nitroindoline

To a solution of 5-ethylindoline (2.5 g, 17 mmol) in H₂SO₄ (98%, 20 mL) was slowly added KNO₃ (1.7 g, 17 mmol) at 0° C. The mixture was stirred at 0-10° C. for 10 minutes. The mixture was then carefully poured into ice, basified with NaOH solution to pH 9, and extracted with ethyl acetate. The combined extracts were washed with brine, dried over Na₂SO₄ and concentrated to dryness. The residue was purified by silica column to give 5-ethyl-6-nitroindoline (1.9 g, 58%).

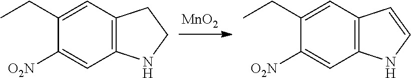

### 5-Ethyl-6-nitro-1H-indole

To a solution of 5-ethyl-6-nitroindoline (1.9 g, 9.9 mmol) in CH₂Cl₂ (30 mL) was added MnO₂ (4.0 g, 46 mmol). The mixture was stirred at ambient temperature for 8 h. The solid was filtered off and the filtrate was concentrated to dryness to give 5-ethyl-6-nitro-1H-indole (1.9 g).

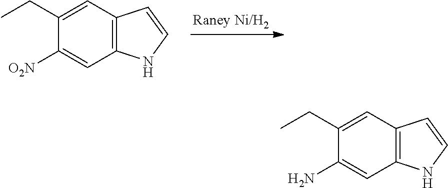

### 5-Ethyl-1H-indol-6-amine

A suspension of 5-ethyl-6-nitro-1H-indole (1.9 g, 10 mmol) and Raney Nickel (1 g) was hydrogenated under H₂ (1 atm) at room temperature for 2 h. The catalyst was filtered off and the filtrate was concentrated to dryness. The residue was purified by silica gel column to give 5-ethyl-1H-indol-6-amine (760 mg, 48% over two steps). ¹H NMR (CDCl₃) δ 7.90 (br s, 1H), 7.41 (s, 1H), 7.00 (s, 1H), 6.78 (s, 2H), 6.39 (s, 1H), 3.39 (br s, 2H), 2.63 (q, J=7.2 Hz, 2H), 1.29 (t, J=6.9 Hz, 3H); MS (ESI) m/e (M+H⁺) 161.1.

### Example 55

### Ethyl 6-amino-1H-indole-4-carboxylate

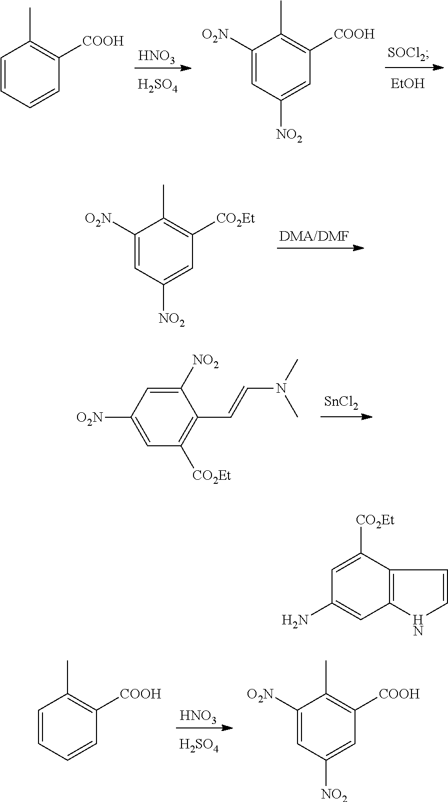

### 2-Methyl-3,5-dinitrobenzoic acid

To a mixture of HNO₃ (95%, 80 mL) and H₂SO₄ (98%, 80 mL) was slowly added 2-methylbenzic acid (50 g, 0.37 mol) at 0° C. After addition, the reaction mixture was stirred below 30° C. for 1.5 h. The mixture then was poured into ice-water and stirred for 15 min. The precipitate was filtered and washed with water to give 2-methyl-3,5-dinitrobenzoic acid (70 g, 84%).

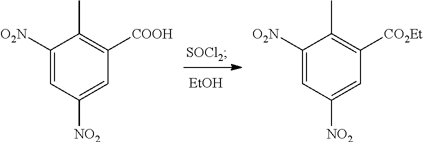 [SMILES: "EtOH" ; Cc1c(C(=O)O)cc([N+](=O)[O-])cc1[N+](=O)[O-] ; "SOCl2;" ; CCOC(=O)c1cc([N+](=O)[O-])cc([N+](=O)[O-])c1C]

### Ethyl 2-methyl-3,5-dinitrobenzoate

A mixture of 2-methyl-3,5-dinitrobenzoic acid (50 g, 0.22 mol) in SOCl₂ (80 mL) was heated at reflux for 4 h and then was concentrated to dryness. The residue was dissolved in CH₂Cl₂ (50 mL), to which EtOH (80 mL) was added and the mixture was stirred at room temperature for 1 h. The mixture was poured into ice-water and extracted with EtOAc (3×100 mL). The combined extracts were washed sat. Na₂CO₃ (80 mL), water (2×100 mL) and brine (100 mL), dried over Na₂SO₄ and concentrated to dryness to give ethyl 2-methyl-3,5-dinitrobenzoate (50 g, 88%)

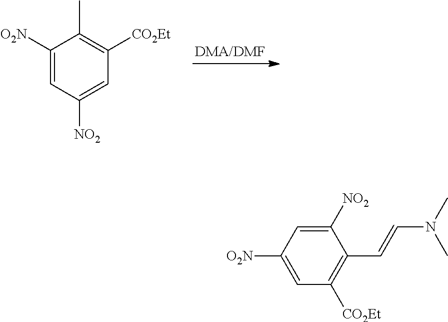 [SMILES: CCOC(=O)c1cc([N+](=O)[O-])cc([N+](=O)[O-])c1C ; "DMA/DMF" ; CCOC(=O)c1cc([N+](=O)[O-])cc([N+](=O)[O-])c1/C=C/N(C)C]

### (E)-Ethyl 2-(2-(dimethylamino)vinyl)-3,5-dinitrobenzoate

A mixture of ethyl 2-methyl-3,5-dinitrobenzoate (35 g, 0.14 mol) and DMA (32 g, 0.27 mol) in DMF (200 mL) was heated at 100° C. for 5 h. The mixture was poured into ice-water and the precipitated solid was filtered and washed with water to give (E)-ethyl 2-(2-(dimethylamino)vinyl)-3,5-dinitrobenzoate (11 g, 48%)

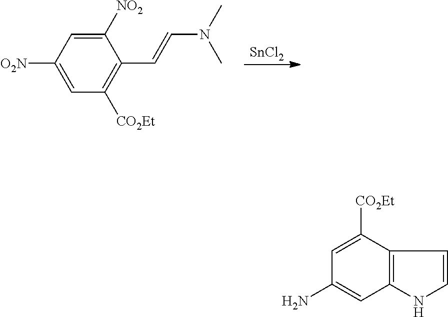

### Ethyl 6-amino-1H-indole-4-carboxylate

A mixture of (E)-ethyl 2-(2-(dimethylamino)vinyl)-3,5-dinitrobenzoate (11 g, 0.037 mol) and SnCl₂ (83 g, 0.37 mol) in ethanol was heated at reflux for 4 h. The mixture was concentrated to dryness and the residue was poured into water and basified using sat. aq. Na₂CO₃ to pH 8. The precipitated solid was filtered and the filtrate was extracted with ethyl acetate (3×100 mL). The combined extracts were washed with water (2×100 mL) and brine (150 mL), dried over Na₂SO₄, and concentrated to dryness. The residue was purified by column on silica gel to give ethyl 6-amino-1H-indole-4-carboxylate (3.0 g, 40%). ¹HNMR (DMSO-d₆) δ 10.76 (br s, 1H), 7.11-7.14 (m, 2H), 6.81-6.82 (m, 1H), 6.67-6.68 (m, 1H), 4.94 (br s, 2H), 4.32-4.25 (q, J=7.2 Hz, 2H), 1.35-1.31 (t, J=7.2, 3H); MS (ESI) m/e (M+H⁺) 205.0.

### Example 56

### 5-Fluoro-1H-indol-6-amine

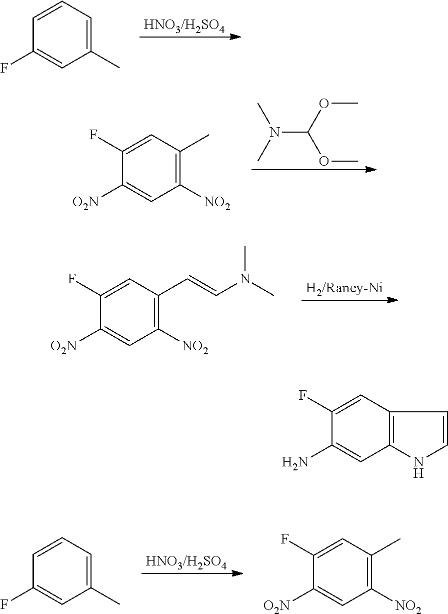

### 1-Fluoro-5-methyl-2,4-dinitrobenzene

To a stirred solution of HNO₃(60 mL) and H₂SO₄ (80 mL) was added dropwise 1-fluoro-3-methylbenzene (28 g, 25 mmol) under ice-cooling at such a rate that the temperature did not rise above 35° C. The mixture was allowed to stir for 30 min at rt and was then poured into ice water (500 mL). The resulting precipitate (a mixture of 1-fluoro-5-methyl-2,4-dinitrobenzene and 1-fluoro-3-methyl-2,4-dinitrobenzene, 32 g, ca. 7:3 ratio) was collected by filtration and purified by recrystallization from 50 mL isopropyl ether to give pure 1-fluoro-5-methyl-2,4-dinitro-benzene as a white solid (18 g, 36%).

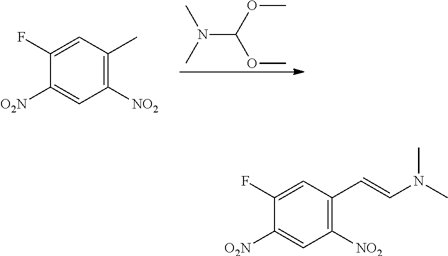

### (E)-2-(5-Fluoro-2,4-dinitrophenyl)-N,N-dimethylethenamine

A mixture of 1-fluoro-5-methyl-2,4-dinitro-benzene (10 g, 50 mmol), DMA (12 g, 100 mmol) and DMF (50 mL) was heated at 100° C. for 4 h. The solution was cooled and poured into water. The precipitated red solid was collected, washed with water, and dried to give (E)-2-(5-fluoro-2,4-dinitrophenyl)-N,N-dimethylethenamine (8.0 g, 63%).

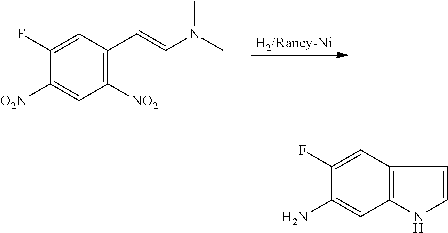

### 5-Fluoro-1H-indol-6-amine

A suspension of (E)-2-(5-fluoro-2,4-dinitrophenyl)-N,N-dimethylethenamine (8.0 g, 31 mmol) and Raney Nickel (8 g) in EtOH (80 mL) was stirred under H₂ (40 psi) at room temperature for 1 h. After filtration, the filtrate was concentrated and the residue was purified by column chromatography (petroleum ether/ethyl acetate=5/1) to give 5-fluoro-1H-indol-6-amine (1.0 g, 16%) as a brown solid. ¹HNMR (DMSO-d₆) δ 10.56 (br s, 1H), 7.07 (d, J=12 Hz, 1H), 7.02 (m, 1H), 6.71 (d, J=8 Hz, 1H), 6.17 (s, 1H), 3.91 (br s, 2H); MS (ESI) m/e (M+H⁺) 150.1.

### Example 57

### 5-Chloro-1H-indol-6-amine

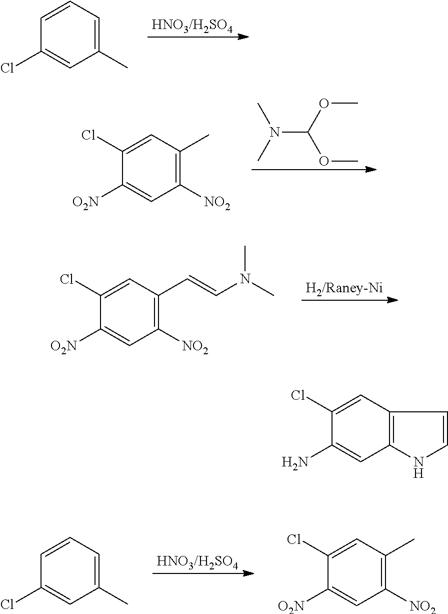

### 1-Chloro-5-methyl-2,4-dinitrobenzene

To a stirred solution of HNO₃ (55 mL) and H₂SO₄ (79 mL) was added dropwise 1-chloro-3-methylbenzene (25.3 g, 200 mmol) under ice-cooling at such a rate that the temperature did not rise above 35° C. The mixture was allowed to stir for 30 min at ambient temperature and was then poured into ice water (500 mL). The resulting precipitate was collected by filtration and purified by recrystallization to give 1-chloro-5-methyl-2,4-dinitrobenzene (26 g, 60%).

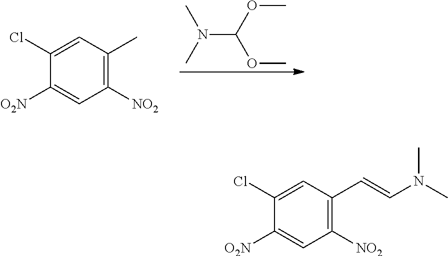

### (E)-2-(5-Chloro-2,4-dinitrophenyl)-N,N-dimethylethenamine

A mixture of 1-chloro-5-methyl-2,4-dinitro-benzene (11.6 g, 50.0 mmol), DMA (11.9 g, 100 mmol) in DMF (50 mL) was heated at 100° C. for 4 h. The solution was cooled and poured into water. The precipitated red solid was collected by filtration, washed with water, and dried to give (E)-2-(5-chloro-2,4-dinitrophenyl)-N,N-dimethylethenamine (9.84 g, 72%).

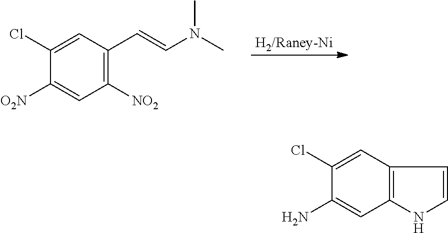

### 5-Chloro-1H-indol-6-amine

A suspension of (E)-2-(5-chloro-2,4-dinitrophenyl)-N,N-dimethylethenamine (9.8 g, 36 mmol) and Raney Nickel (9.8 g) in EtOH (140 mL) was stirred under H₂ (1 atm) at room temperature for 4 h. After filtration, the filtrate was concentrated and the residue was purified by column chromatograph (petroleum ether/ethyl acetate=10:1) to give 5-chloro-1H-indol-6-amine (0.97 g, 16%) as a gray powder. ¹HNMR (CDCl₃) δ 7.85 (br s, 1H), 7.52 (s, 1H), 7.03 (s, 1H), 6.79 (s, 1H), 6.34 (s, 1H), 3.91 (br s, 1H); MS (ESI) m/e (M+H⁺) 166.0.

### Example 58

### Ethyl 6-amino-1H-indole-7-carboxylate

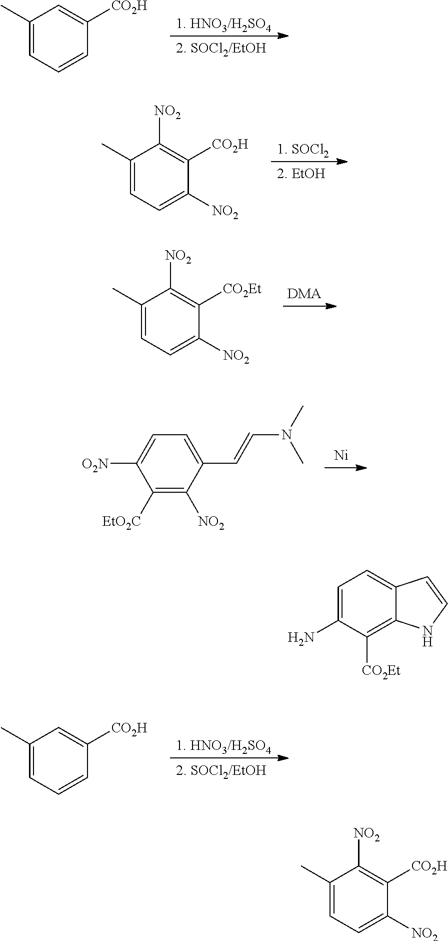

### 3-Methyl-2,6-dinitrobenzoic acid

To a mixture of HNO₃ (95%, 80 mL) and H₂SO₄(98%, 80 mL) was slowly added 3-methylbenzic acid (50 g, 0.37 mol) at 0° C. After addition, the mixture was stirred below 30° C. for 1.5 hours. The mixture was then poured into ice-water and stirred for 15 min. The precipitate solid was filtered and washed with water to give a mixture of 3-methyl-2,6-dinitro-benzoic acid and 5-methyl-2,4-dinitrobenzoic acid (70 g, 84%). To a solution of this mixture (70 g, 0.31 mol) in EtOH (150 mL) was added dropwise SOCl₂ (54 g, 0.45 mol). The mixture was heated at reflux for 2 h before being concentrated to dryness under reduced pressure. The residue was partitioned between EtOAc (100 mL) and aq. Na₂CO₃ (10%, 120 mL). The organic layer was washed with brine (50 mL), dried over Na₂SO₄, and concentrated to dryness to obtain ethyl 5-methyl-2,4-dinitrobenzoate (20 g), which was placed aside. The aqueous layer was acidified by HCl to pH 2˜3 and the precipitated solid was filtered, washed with water, and dried in air to give 3-methyl-2,6-dinitrobenzoic acid (39 g, 47%).

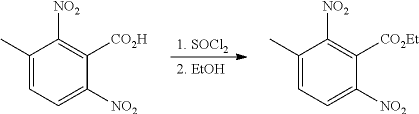 [SMILES: Cc1ccc([N+](=O)[O-])c(C(=O)O)c1[N+](=O)[O-] ; "1. SOCl2" ; "2. EtOH" ; CCOC(=O)c1c([N+](=O)[O-])ccc(C)c1[N+](=O)[O-]]

### Ethyl 3-methyl-2,6-dinitrobenzoate

A mixture of 3-methyl-2,6-dinitrobenzoic acid (39 g, 0.15 mol) and SOCl₂ (80 mL) was heated at reflux 4 h. The excess SOCl₂ was evaporated off under reduced pressure and the residue was added dropwise to a solution of EtOH (100 mL) and Et₃N (50 mL). The mixture was stirred at 20° C. for 1 h and then concentrated to dryness. The residue was dissolved in EtOAc (100 mL), washed with Na₂CO₃ (10%, 40 mL×2), water (50 mL×2) and brine (50 mL), dried over Na₂SO₄ and concentrated to give ethyl 3-methyl-2,6-dinitrobenzoate (20 g, 53%).

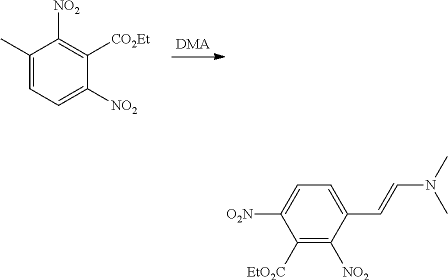

### (E)-Ethyl 3-(2-(dimethylamino)vinyl)-2,6-dinitrobenzoate

A mixture of ethyl 3-methyl-2,6-dinitrobenzoate (35 g, 0.14 mol) and DMA (32 g, 0.27 mol) in DMF (200 mL) was heated at 100° C. for 5 h. The mixture was poured into ice water. The precipitated solid was filtered and washed with water to give (E)-ethyl 3-(2-(dimethylamino)vinyl)-2,6-dinitrobenzoate (25 g, 58%).

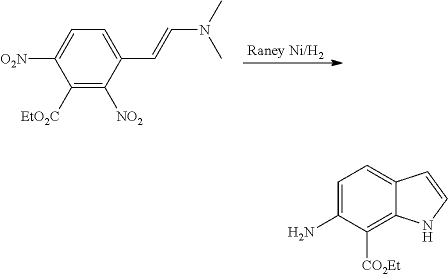

### Ethyl 6-amino-1H-indole-7-carboxylate

A mixture of (E)-ethyl 3-(2-(dimethylamino)vinyl)-2,6-dinitrobenzoate (30 g, 0.097 mol) and Raney Nickel (10 g) in EtOH (1000 mL) was hydrogenated at room temperature under 50 psi for 2 h. The catalyst was filtered off and the filtrate was concentrated to dryness. The residue was purified by column on silica gel to give ethyl 6-amino-1H-indole-7-carboxylate as an off-white solid (3.2 g, 16%).

NMR (DMSO-d₆) δ 10.38 (s, 1H), 7.42 (d, J=8.7 Hz, 1H), 6.98 (t, J=3.0 Hz, 1H), 6.65 (s, 2H), 6.48 (d, J=8.7 Hz, 1H), 6.27-6.26 (m, 1H), 4.38 (q, J=7.2 Hz, 2H), 1.35 (t, J=7.2 Hz, 3H).

### Example 59

### Ethyl 6-amino-1H-indole-5-carboxylate

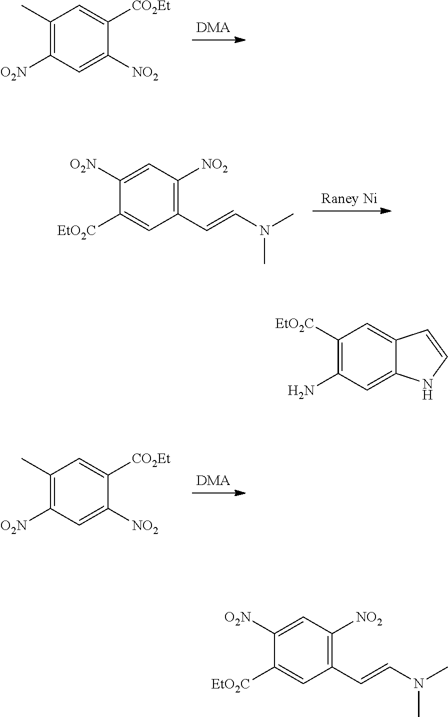

### (E)-Ethyl 5-(2-(dimethylamino)vinyl)-2,4-dinitrobenzoate

A mixture of ethyl 5-methyl-2,4-dinitrobenzoate (39 g, 0.15 mol) and DMA (32 g, 0.27 mol) in DMF (200 mL) was heated at 100° C. for 5 h. The mixture was poured into ice water and the precipitated solid was filtered and washed with water to afford (E)-ethyl 5-(2-(dimethylamino)vinyl)-2,4-dinitrobenzoate (15 g, 28%).

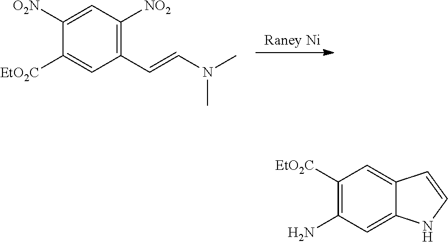

### Ethyl 6-amino-1H-indole-5-carboxylate

A mixture of (E)-ethyl 5-(2-(dimethylamino)vinyl)-2,4-dinitrobenzoate (15 g, 0.050 mol) and Raney Nickel (5 g) in EtOH (500 mL) was hydrogenated at room temperature under 50 psi of hydrogen for 2 h. The catalyst was filtered off and the filtrate was concentrated to dryness. The residue was purified by column on silica gel to give ethyl 6-amino-1H-indole-5-carboxylate (3.0 g, 30%). ¹H NMR (DMSO-d₆) δ 10.68 (s, 1H), 7.99 (s, 1H), 7.01-7.06 (m, 1H), 6.62 (s, 1H), 6.27-6.28 (m, 1H), 6.16 (s, 2H), 4.22 (q, J=7.2 Hz, 2H), 1.32-1.27 (t, J=7.2 Hz, 3H).

### Example 60

### 5-tert-Butyl-1H-indol-6-amine

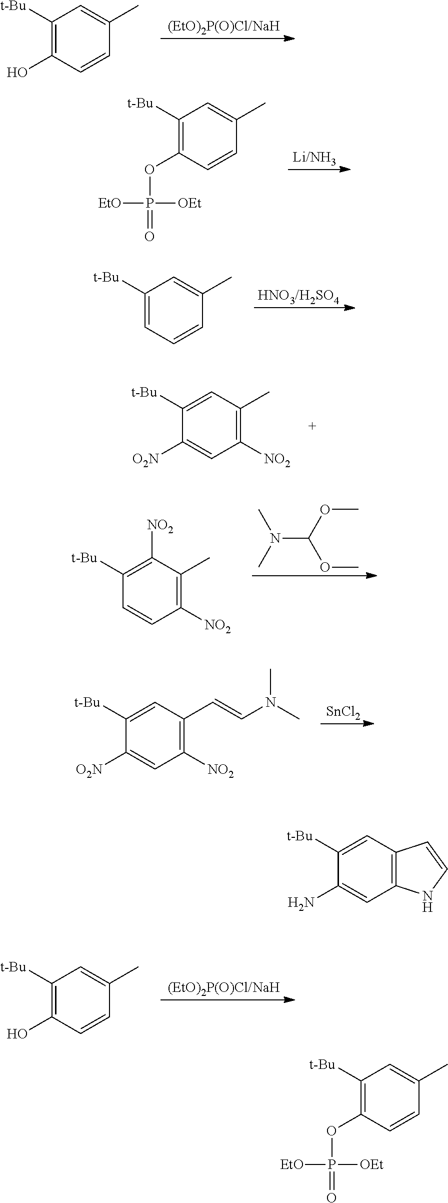

### 2-tert-Butyl-4-methylphenyl diethyl phosphate

To a suspension of NaH (60% in mineral oil, 8.4 g, 0.21 mol) in THF (200 mL) was added dropwise a solution of 2-tert-butyl-4-methylphenol (33 g, 0.20 mol) in THF (100 mL) at 0° C. The mixture was stirred at 0° C. for 15 min and then phosphorochloridic acid diethyl ester (37 g, 0.21 mol) was added dropwise at 0° C. After addition, the mixture was stirred at ambient temperature for 30 min. The reaction was quenched with sat. NH₄Cl (300 mL) and then extracted with Et₂O (350 mL×2). The combined organic layers were washed with brine, dried over anhydrous Na₂SO₄, and then evaporated under vacuum to give 2-tert-butyl-4-methylphenyl diethyl phosphate (contaminated with mineral oil) as a colorless oil (60 g, ˜100%), which was used directly in the next step.

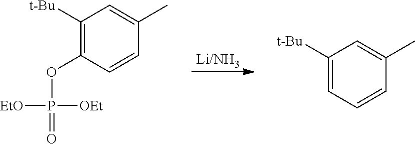 [SMILES: CCOP(=O)(OCC)Oc1ccc(C)cc1C(C)(C)C ; "Li/NH3" ; Cc1cccc(C(C)(C)C)c1]

### 1-tert-Butyl-3-methylbenzene

To NH₃ (liquid, 1000 mL) was added a solution of 2-tert-butyl-4-methylphenyl diethyl phosphate (60 g, crude from last step, about 0.2 mol) in Et₂O (anhydrous, 500 mL) at −78° C. under N₂ atmosphere. Lithium metal was added to the solution in small pieces until the blue color persisted. The reaction mixture was stirred at −78° C. for 15 min and then was quenched with sat. NH₄Cl until the mixture turned colorless. Liquid NH₃ was evaporated and the residue was dissolved in water. The mixture was extracted with Et₂O (400 mL×2). The combined organics were dried over Na₂SO₄ and evaporated to give 1-tert-butyl-3-methylbenzene (contaminated with mineral oil) as a colorless oil (27 g, 91%), which was used directly in next step.

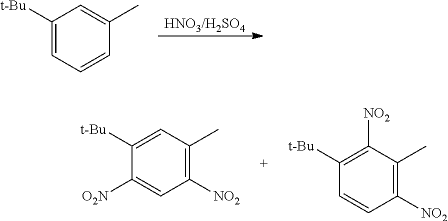 [SMILES: Cc1cccc(C(C)(C)C)c1 ; Cc1cc(C(C)(C)C)c([N+](=O)[O-])cc1[N+](=O)[O-] ; Cc1c([N+](=O)[O-])ccc(C(C)(C)C)c1[N+](=O)[O-]]

### 1-tert-Butyl-5-methyl-2,4-dinitrobenzene and 1-tert-butyl-3-methyl-2,4-dinitro-benzene

To HNO₃ (95%, 14 mL) was added H₂SO₄ (98%, 20 mL) at 0° C. and then 1-tert-butyl-3-methylbenzene (7.4 g, ˜50 mmol, crude from last step) dropwise to the with the temperature being kept below 30° C. The mixture was stirred at ambient temperature for 30 min, poured onto crushed ice (100 g), and extracted with EtOAc (50 mL three times). The combined organic layers were washed with water and brine, before being evaporated to give a brown oil, which was purified by column chromatography to give a mixture of 1-tert-butyl-5-methyl-2,4-dinitrobenzene and 1-tert-butyl-3-methyl-2,4-dinitrobenzene (2:1 by NMR) as a yellow oil (9.0 g, 61%).

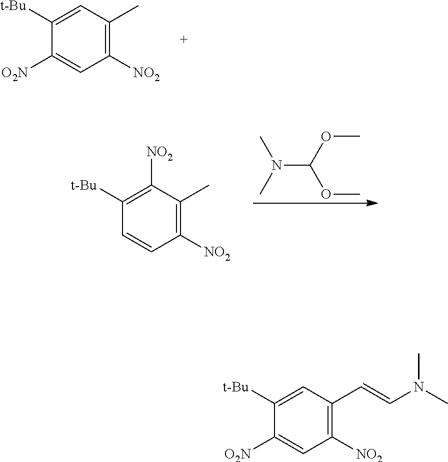

### (E)-2-(5-tert-Butyl-2,4-dinitrophenyl)-N,N-dimethylethenamine

A mixture of 1-tert-butyl-5-methyl-2,4-dinitrobenzene and 1-tert-butyl-3-methyl-2,4-dinitrobenzene (9.0 g, 38 mmol, 2:1 by NMR) and DMA (5.4 g, 45 mmol) in DMF (50 mL) was heated at reflux for 2 h before being cooled to room temperature. The reaction mixture was poured into water-ice and extracted with EtOAc (50 mL three times). The combined organic layers were washed with water and brine, before being evaporated to give a brown oil, which was purified by column to give (E)-2-(5-tert-butyl-2,4-dinitrophenyl)-N,N-dimethylethen-amine (5.0 g, 68%).

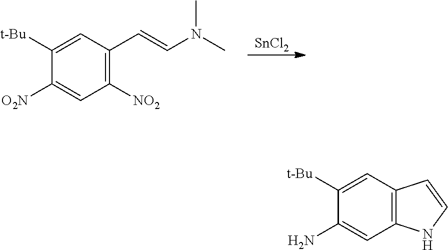

### 5-tert-Butyl-1H-indol-6-amine

A solution of (E)-2-(5-tert-butyl-2,4-dinitrophenyl)-N,N-dimethylethen-amine (5.3 g, 18 mmol) and tin (II) chloride dihydrate (37 g, 0.18 mol) in ethanol (200 mL) was heated at reflux overnight. The mixture was cooled to room temperature and the solvent was removed under vacuum. The residual slurry was diluted with water (500 mL) and was basifed with 10% aq. Na₂CO₃ to pH 8. The resulting suspension was extracted with ethyl acetate (3×100 mL). The ethyl acetate extract was washed with water and brine, dried over Na₂SO₄, and concentrated. The residual solid was washed with CH₂Cl₂ to afford a yellow powder, which was purified by column chromatography to give 5-tert-butyl-1H-indol-6-amine (0.40 g, 12%). ¹H NMR (DMSO d₆) δ 10.34 (br s, 1H), 7.23 (s, 1H), 6.92 (s, 1H), 6.65 (s, 1H), 6.14 (s, 1H), 4.43 (br s, 2H), 2.48 (s, 9H); MS (ESI) m/e (M+H⁴) 189.1.

### General Procedure IV

### Synthesis of Acylaminoindoles

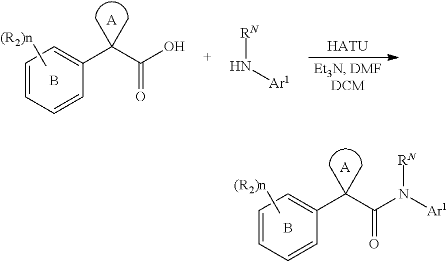

One equivalent of the appropriate carboxylic acid and one equivalent of the appropriate amine were dissolved in N,N-dimethylformamide (DMF) containing triethylamine (3 equivalents). 047-Azabenzotriazol-1-yl)-N,N,N′,N′-tetramethyluronium hexafluorophosphate (HATU) was added and the solution was allowed to stir. The crude product was purified by reverse-phase preparative liquid chromatography to yield the pure product.

### Example 61

### N-(2-tert-Butyl-1H-indol-5-yl)-1-(4-methoxyphenyl)-cyclopropanecarboxamide

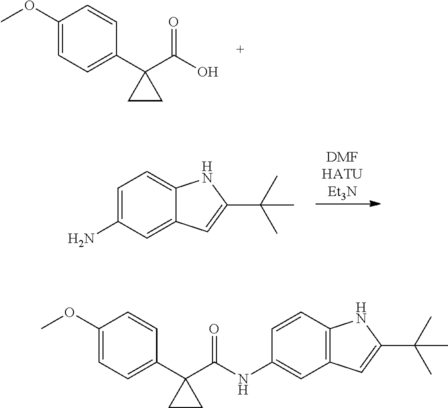 [SMILES: COc1ccc(C2(C(=O)O)CC2)cc1 ; CC(C)(C)c1cc2cc(N)ccc2[nH]1 ; COc1ccc(C2(C(=O)Nc3ccc4[nH]c(C(C)(C)C)cc4c3)CC2)cc1]

2-tert-Butyl-1H-indol-5-amine (19 mg, 0.10 mmol) and 1-(4-methoxyphenyl)-cyclopropanecarboxylic acid (19 mg, 0.10 mmol) were dissolved in N,N-dimethylformamide (1.00 mL) containing triethylamine (28 μL, 0.20 mmol). 0-(7-Azabenzotriazol-1-yl)-N,N,N′,N′-tetramethyluronium hexafluorophosphate (42 mg, 0.11 mmol) was added to the mixture and the resulting solution was allowed to stir for 3 hours. The crude reaction mixture was filtered and purified by reverse phase HPLC. ESI-MS m/z calc. 362.2. found 363.3 (M+1)⁺; Retention time 3.48 minutes.

### General Procedure V

### Synthesis of Acylaminoindoles

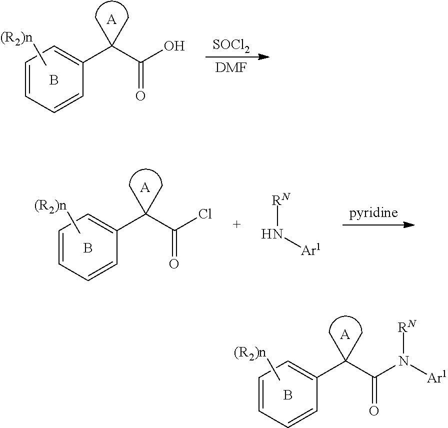

One equivalent of the appropriate carboxylic acid was placed in an oven-dried flask under nitrogen. A minimum (3 equivalents) of thionyl chloride and a catalytic amount of and N,N-dimethylformamide were added and the solution was allowed to stir for 20 minutes at 60° C. The excess thionyl chloride was removed under vacuum and the resulting solid was suspended in a minimum of anhydrous pyridine. This solution was slowly added to a stirred solution of one equivalent the appropriate amine dissolved in a minimum of anhydrous pyridine. The resulting mixture was allowed to stir for 15 hours at 110° C. The mixture was evaporated to dryness, suspended in dichloromethane, and then extracted three times with 1N HCl. The organic layer was then dried over sodium sulfate, evaporated to dryness, and then purified by column chromatography.

### Example 62

### Ethyl 5-(1-(benzo[d][1,3]dioxol-5-yl)cyclopropanecarboxamido)-1H-indole-2-carboxylate (Compound. 28)

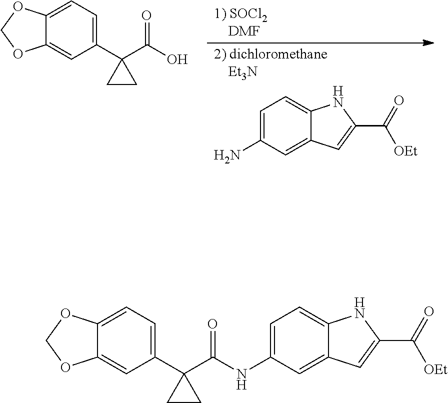 [SMILES: O=C(O)C1(c2ccc3c(c2)OCO3)CC1 ; CCOC(=O)c1cc2cc(N)ccc2[nH]1 ; CCOC(=O)c1cc2cc(NC(=O)C3(c4ccc5c(c4)OCO5)CC3)ccc2[nH]1]

1-Benzo[1,3]dioxol-5-yl-cyclopropanecarboxylic acid (2.07 g, 10.0 mmol) was dissolved in thionyl chloride (2.2 mL) under N₂. N,N-dimethylformamide (0.3 mL) was added and the solution was allowed to stir for 30 minutes. The excess thionyl chloride was removed under vacuum and the resulting solid was dissolved in anhydrous dichloromethane (15 mL) containing triethylamine (2.8 mL, 20.0 mmol). Ethyl 5-amino-1H-indole-2-carboxylate (2.04 g, 10.0 mmol) in 15 mL of anhydrous dichloromethane was slowly added to the reaction. The resulting solution was allowed to stir for 1 hour. The reaction mixture was diluted to 50 mL with dichloromethane and washed three times with 50 mL of 1N HCl, saturated aqueous sodium bicarbonate, and saturated aqueous sodium chloride. The organic layer was dried over sodium sulfate and evaporated to dryness to yield ethyl 5-(1-(benzo[d][1,3]dioxol-5-yl)cyclopropanecarboxamido)-1H-indole-2-carboxylate as a gray solid (3.44 g, 88%). ESI-MS m/z calc. 392.4. found 393.1 (M+1)⁺ Retention time 3.17 minutes. ¹H NMR (400 MHz, DMSO-d6) δ 11.80 (s, 1H), 8.64 (s, 1H), 7.83 (m, 1H), 7.33-7.26 (m, 2H), 7.07 (m, 1H), 7.02 (m, 1H), 6.96-6.89 (m, 2H), 6.02 (s, 2H), 4.33 (q, J=7.1 Hz, 2H), 1.42-1.39 (m, 2H), 1.33 (t, J=7.1 Hz, 3H), 1.06-1.03 (m, 2H).

### Example 63

### 1-(Benzo[d][1,3]dioxol-5-yl)-N-(2-tert-butyl-1H-indol-5-yl)cyclopropanecarboxamide

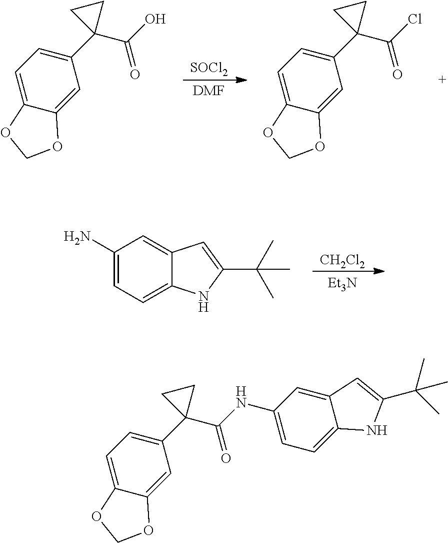

1-Benzo[1,3]dioxol-5-yl-cyclopropanecarboxylic acid (1.09 g, 5.30 mmol) was dissolved in 2 mL of thionyl chloride under nitrogen. A catalytic amount (0.3 mL) of N,N-dimethylformamide (DMF) was added and the reaction mixture was stirred for 30 minutes. The excess thionyl chloride was evaporated and the resulting residue was dissolved in 15 mL of dichloromethane. This solution was slowly added to a solution of 2-tert-butyl-1H-indol-5-amine (1.0 g, 5.3 mmol) in 10 mL of dichloromethane containing triethylamine (1.69 mL, 12.1 mmol). The resulting solution was allowed to stir for 10 minutes. The solvent was evaporated to dryness and the crude reaction mixture was purified by silica gel column chromatography using a gradient of 5-50% ethyl acetate in hexanes. The pure fractions were combined and evaporated to dryness to yield a pale pink powder (1.24 g 62%). ESI-MS m/z calc. 376.18. found 377.3 (M+1)⁺. Retention time of 3.47 minutes. ¹H NMR (400 MHz, DMSO) δ 10.77 (s, 1H), 8.39 (s, 1H), 7.56 (d, J=1.4 Hz, 1H), 7.15 (d, J=8.6 Hz, 1H), 7.05-6.87 (m, 4H), 6.03 (s, 3H), 1.44-1.37 (m, 2H), 1.33 (s, 9H), 1.05-1.00 (m, 2H).

### Example 64

### 1-(Benzo[d][1,3]dioxol-5-yl)-N-(1-methyl-2-(1-methylcyclopropyl)-1H-indol-5-yl)cyclopropanecarboxamide

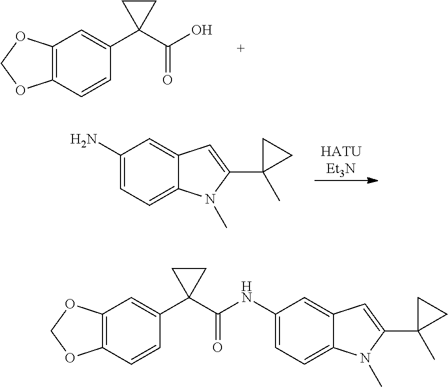

1-Methyl-2-(1-methylcyclopropyl)-1H-indol-5-amine (20.0 mg, 0.100 mmol) and 1-(benzo[d][1,3]dioxol-5-yl)cyclopropanecarboxylic acid (20.6 mg, 0.100 mmol) were dissolved in N,N-dimethylformamide (1 mL) containing triethylamine (42.1 μL, 0.300 mmol) and a magnetic stir bar. 047-Azabenzotriazol-1-yl)-N,N,N′,N′-tetramethyluronium hexafluorophosphate (42 mg, 0.11 mmol) was added to the mixture and the resulting solution was allowed to stir for 6 h at 80° C. The crude product was then purified by preparative HPLC utilizing a gradient of 0-99% acetonitrile in water containing 0.05% trifluoroacetic acid to yield 1-(benzo[d][1,3]dioxol-5-yl)-N-(1-methyl-2-(1-methylcyclopropyl)-1H-indol-5-yl)cyclopropanecarboxamide. ESI-MS m/z calc. 388.2. found 389.2 (M+1)⁺. Retention time of 3.05 minutes.

### Example 65

### 1-(Benzo[d][1,3]dioxol-5-yl)-N-(1,1-dimethyl-2,3-dihydro-1H-pyrrolo[1,2-a]indol-7-yl)cyclopropanecarboxamide

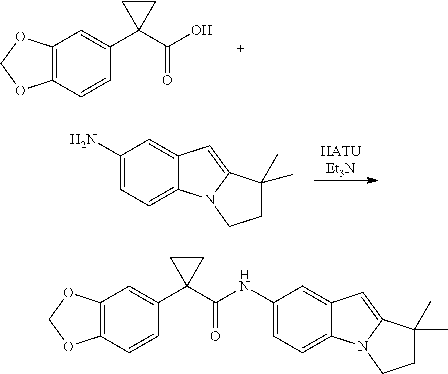

1,1-Dimethyl-2,3-dihydro-1H-pyrrolo[1,2-a]indol-7-amine (40.0 mg, 0.200 mmol) and 1-(benzo[d][1,3]dioxol-5-yl)cyclopropanecarboxylic acid (41.2 mg, 0.200 mmol) were dissolved in N,N-dimethylformamide (1 mL) containing triethylamine (84.2 μL, 0.600 mmol) and a magnetic stir bar. O-(7-Azabenzotriazol-1-yl)-N,N,N′,N′-tetramethyluronium hexafluorophosphate (84 mg, 0.22 mmol) was added to the mixture and the resulting solution was allowed to stir for 5 minutes at room temperature. The crude product was then purified by preparative HPLC utilizing a gradient of 0-99% acetonitrile in water containing 0.05% trifluoroacetic acid to yield 1-(benzo[d][1,3]dioxol-5-yl)-N-(1,1-dimethyl-2,3-dihydro-1H-pyrrolo[1,2-a]-indol-7-yl)cyclopropanecarboxamide. ESI-MS m/z calc. 388.2. found 389.2 (M+1)⁺. Retention time of 2.02 minutes. ¹H NMR (400 MHz, DMSO-d6) δ 8.41 (s, 1H), 7.59 (d, J=1.8 Hz, 1H), 7.15 (d, J=8.6 Hz, 1H), 7.06-7.02 (m, 2H), 6.96-6.90 (m, 2H), 6.03 (s, 2H), 5.98 (d, J=0.7 Hz, 1H), 4.06 (t, J=6.8 Hz, 2H), 2.35 (t, J=6.8 Hz, 2H), 1.42-1.38 (m, 2H), 1.34 (s, 6H), 1.05-1.01 (m, 2H).

### Example 66

### Methyl 5-(1-(benzo[d][1,3]dioxol-5-yl)cyclopropanecarboxamido)-2-tert-butyl-1H-indole-7-carboxylate

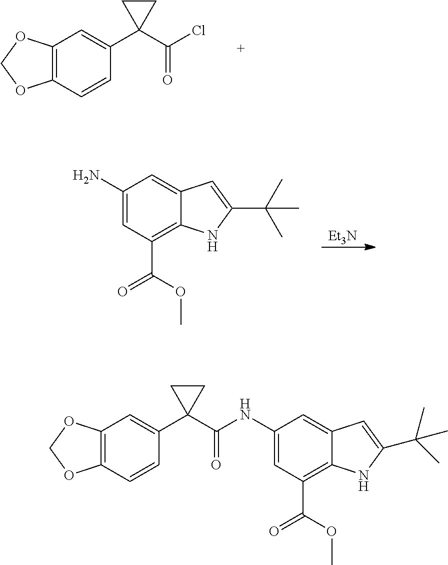 [SMILES: "Et3N" ; O=C(Cl)C1(c2ccc3c(c2)OCO3)CC1 ; COC(=O)c1cc(N)cc2cc(C(C)(C)C)[nH]c12 ; COC(=O)c1cc(NC(=O)C2(c3ccc4c(c3)OCO4)CC2)cc2cc(C(C)(C)C)[nH]c12]

1-(Benzo[d][1,3]dioxol-5-yl)cyclopropanecarbonyl chloride (45 mg, 0.20 mmol) and methyl 5-amino-2-tert-butyl-1H-indole-7-carboxylate (49.3 mg, 0.200 mmol) were dissolved in N,N-dimethylformamide (2 mL) containing a magnetic stir bar and triethylamine (0.084 mL, 0.60 mmol). The resulting solution was allowed to stir for 10 minutes at room temperature. The crude product was then purified by preparative HPLC using a gradient of 0-99% acetonitrile in water containing 0.05% trifluoroacetic acid to yield methyl 5-(1-(benzo[d][1,3]dioxol-5-yl)cyclopropanecarbox-amido)-2-tert-butyl-1H-indole-7-carboxylate. ESI-MS m/z calc. 434.2. found 435.5. (M+1)⁺. Retention time of 2.12 minutes.

### Example 67

### 1-(Benzo[d][1,3]dioxol-5-yl)-N-(2-(1-hydroxy-2-methylpropan-2-yl)-1H-indol-5-yl)cyclopropanecarboxamide

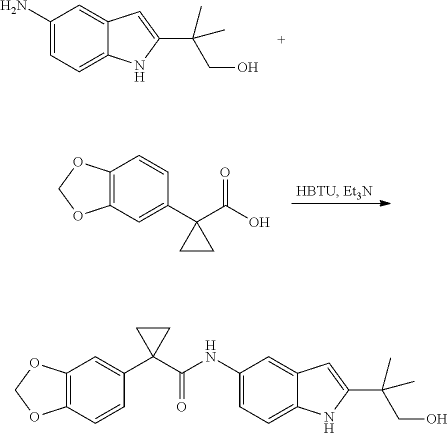 [SMILES: CC(C)(CO)c1cc2cc(N)ccc2[nH]1 ; O=C(O)C1(c2ccc3c(c2)OCO3)CC1 ; CC(C)(CO)c1cc2cc(NC(=O)C3(c4ccc5c(c4)OCO5)CC3)ccc2[nH]1]

To a solution of 1-(benzo[d][1,3]dioxol-5-yl)cyclopropanecarboxylic acid (0.075 g, 0.36 mmol) in acetonitrile (1.5 mL) were added HBTU (0.138 g, 0.36 mmol) and Et₃N (152 μL, 1.09 mmol) at room temperature. The mixture was stirred at room temperature for 10 minutes before a solution of 2-(5-amino-1H-indol-2-yl)-2-methylpropan-1-ol (0.074 g, 0.36 mmol) in acetonitrile (1.94 mL) was added. After addition, the reaction mixture was stirred at room temperature for 3 h. The solvent was evaporated under reduced pressure and the residue was dissolved in dichloromethane. The organic layer was washed with 1N HCl (1×3 mL) and saturated aqueous NaHCO₃ (1×3 mL). The organic layer was dried over Na₂SO₄, filtered and evaporated under reduced pressure. The crude material was purified by column chromatography on silica gel (ethyl acetate/hexane=1/1) to give 1-(benzo[d][1,3]dioxol-5-yl)-N-(2-(1-hydroxy-2-methylpropan-2-yl)-1H-indol-5-yl)cyclopropanecarboxamide (0.11 g, 75%). ¹H NMR (400 MHz, DMSO-d6) δ 10.64 (s, 1H), 8.38 (s, 1H), 7.55 (s, 1H), 7.15 (d, J=8.6 Hz, 1H), 7.04-6.90 (m, 4H), 6.06 (s, 1H), 6.03 (s, 2H), 4.79 (t, J=2.7 Hz, 1H), 3.46 (d, J=0.0 Hz, 2H), 1.41-1.39 (m, 2H), 1.26 (s, 6H), 1.05-1.02 (m, 2H).

### Example 67

### 1-(Benzo[d][1,3]dioxol-5-yl)-N-(2,3,4,9-tetrahydro-1H-carbazol-6-yl)cyclopropanecarboxamide

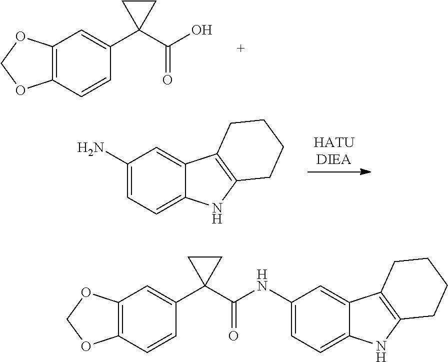 [SMILES: O=C(O)C1(c2ccc3c(c2)OCO3)CC1 ; Nc1ccc2[nH]c3c(c2c1)CCCC3 ; O=C(Nc1ccc2[nH]c3c(c2c1)CCCC3)C1(c2ccc3c(c2)OCO3)CC1]

2,3,4,9-Tetrahydro-1H-carbazol-6-amine (81.8 mg, 0.439 mmol) and 1-(benzo[d][1,3]dioxol-5-yl)cyclopropanecarboxylic acid (90.4 mg, 0.439 mmol) were dissolved in acetonitrile (3 mL) containing diisopropylethylamine (0.230 mL, 1.32 mmol) and a magnetic stir bar. 047-Azabenzotriazol-1-yl)-N,N,N′,N′-tetramethyluronium hexafluorophosphate (183 mg, 0.482 mmol) was added to the mixture and the resulting solution was allowed to stir for 16 h at 70° C. The solvent was evaporated and the crude product was then purified on 40 g of silica gel utilizing a gradient of 5-50% ethyl acetate in hexanes to yield 1-(benzo[d][1,3]dioxol-5-yl)-N-(2,3,4,9-tetrahydro-1H-carbazol-6-yl)cyclopropanecarboxamide as a beige powder (0.115 g, 70%) after drying. ESI-MS m/z calc. 374.2. found 375.3 (M+1)⁺. Retention time of 3.43 minutes. ¹H NMR (400 MHz, DMSO-d6) δ 10.52 (s, 1H), 8.39 (s, 1H), 7.46 (d, J=1.8 Hz, 1H), 7.10-6.89 (m, 5H), 6.03 (s, 2H), 2.68-2.65 (m, 2H), 2.56-2.54 (m, 2H), 1.82-1.77 (m, 4H), 1.41-1.34 (m, 2H), 1.04-0.97 (m, 2H).

### Example 69

### tert-Butyl 4-(5-(1-(benzo[d][1,3]dioxol-5-yl)cyclopropanecarbox-amido)-1H-indol-2-yl)piperidine-1-carboxylate

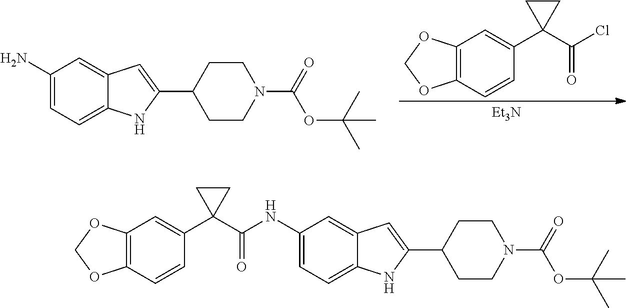 [SMILES: "Et3N" ; CC(C)(C)OC(=O)N1CCC(c2cc3cc(N)ccc3[nH]2)CC1 ; O=C(Cl)C1(c2ccc3c(c2)OCO3)CC1 ; CC(C)(C)OC(=O)N1CCC(c2cc3cc(NC(=O)C4(c5ccc6c(c5)OCO6)CC4)ccc3[nH]2)CC1]

1-(Benzo[d][1,3]dioxol-5-yl)cyclopropanecarbonyl chloride (43 mg, 0.19 mmol) and tert-butyl 4-(5-amino-1H-indol-2-yl)piperidine-1-carboxylate (60 mg, 0.19 mmol) were dissolved in dichloromethane (1 mL) containing a magnetic stir bar and triethylamine (0.056 mL, 0.40 mmol). The resulting solution was allowed to stir for two days at room temperature. The crude product was then evaporated to dryness, dissolved in a minimum of N,N-dimethylformamide, and then purified by preparative HPLC using a gradient of 0-99% acetonitrile in water containing 0.05% trifluoroacetic acid to yield tert-butyl 4-(5-(1-(benzo[d][1,3]dioxol-5-yl)cyclopropanecarboxamido)-1H-indol-2-yl)piperidine-1-carboxylate. ESI-MS m/z calc. 503.2. found 504.5. (M+1)⁺. Retention time of 1.99 minutes.

### Example 70

### Ethyl 2-(5-(1-(benzo[d][1,3]dioxol-5-yl)cyclopropanecarboxamido)-1H-indol-2-yl)propanoate

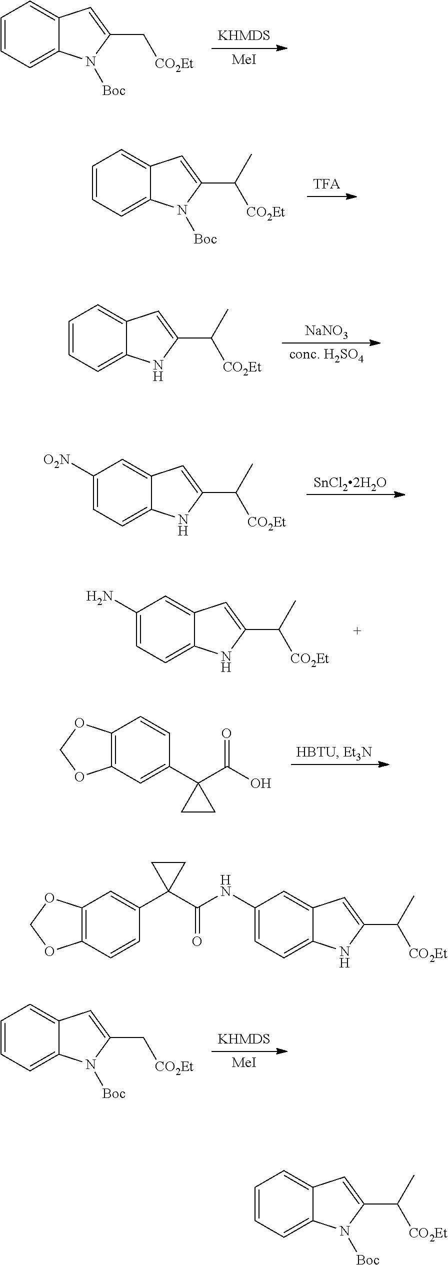

### tert-Butyl 2-(1-ethoxy-1-oxopropan-2-yl)-1H-indole-1-carboxylate

tert-Butyl 2-(2-ethoxy-2-oxoethyl)-1H-indole-1-carboxylate (3.0 g, 9.9 mmol) was added to anhydrous THF (29 mL) and cooled to −78° C. A 0.5M solution of potassium hexamethyldisilazane (20 mL, 9.9 mmol) was added slowly such that the internal temperature stayed below −60° C. Stirring was continued for 1 h at −78° C. Methyl iodide (727 μL, 11.7 mmol) was added to the mixture. The mixture was stirred for 30 minutes at room temperature. The mixture was quenched with sat. aq. ammonium chloride and partitioned between water and dichloromethane. The aqueous phase was extracted with dichloromethane and the combined organic phases were dried over Na₂SO₄ and evaporated under reduced pressure. The residue was purified by column chromatography on silica gel (ethylacetate/hexane=1/9) to give tert-butyl 2-(1-ethoxy-1-oxopropan-2-yl)-1H-indole-1-carboxylate (2.8 g, 88%).

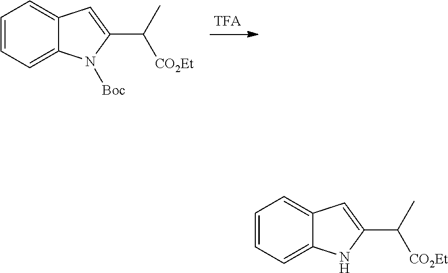 [SMILES: CCOC(=O)C(C)c1cc2ccccc2n1C(=O)OC(C)(C)C ; CCOC(=O)C(C)c1cc2ccccc2[nH]1]

### Ethyl 2-(1H-indol-2-yl)propanoate

tert-Butyl 2-(1-ethoxy-1-oxopropan-2-yl)-1H-indole-1-carboxylate (2.77 g, 8.74 mmol) was dissolved in dichloromethane (25 mL) before TFA (9.8 mL) was added. The mixture was stirred for 1.5 h at room temperature. The mixture was evaporated to dryness, taken up in dichloromethane and washed with sat. aq. sodium bicarbonate, water, and brine. The product was purified by column chromatography on silica gel (0-20% EtOAc in hexane) to give ethyl 2-(1H-indol-2-yl)propanoate (0.92 g, 50%).

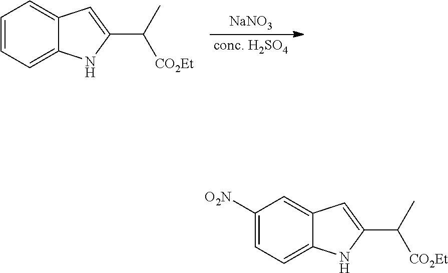

### Ethyl 2-(5-nitro-1H-indol-2-yl)propanoate

Ethyl 2-(1H-indol-2-yl)propanoate (0.91 g, 4.2 mmol) was dissolved in concentrated sulfuric acid (3.9 mL) and cooled to −10° C. (salt/ice-mixture). A solution of sodium nitrate (0.36 g, 4.2 mmol) in concentrated sulfuric acid (7.8 mL) was added dropwise over 35 min. Stirring was continued for another 30 min at −10° C. The mixture was poured into ice and the product was extracted with ethyl acetate. The combined organic phases were washed with a small amount of sat. aq. sodium bicarbonate. The product was purified by column chromatography on silica gel (5-30% EtOAc in hexane) to give ethyl 2-(5-nitro-1H-indol-2-yl)propanoate (0.34 g, 31%).

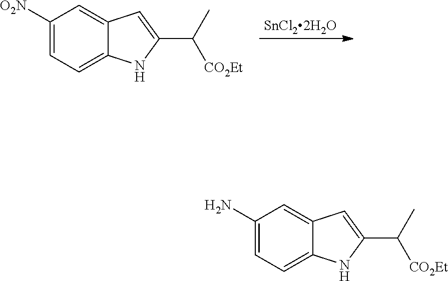 [SMILES: CCOC(=O)C(C)c1cc2cc([N+](=O)[O-])ccc2[nH]1 ; "SnCl2·2H2O" ; CCOC(=O)C(C)c1cc2cc(N)ccc2[nH]1]

### Ethyl 2-(5-amino-1H-indol-2-yl)propanoate

To a solution of ethyl 2-(5-nitro-1H-indol-2-yl)propanoate (0.10 g, 0.38 mmol) in ethanol (4 mL) was added tin chloride dihydrate (0.431 g, 1.91 mmol). The mixture was heated in the microwave at 120° C. for 1 h. The mixture was diluted with ethyl acetate before water and saturated aqueous NaHCO₃ were added. The reaction mixture was filtered through a plug of celite using ethyl acetate. The organic layer was separated from the aqueous layer. The organic layer was dried over Na₂SO₄, filtered and evaporated under reduced pressure to give ethyl 2-(5-amino-1H-indol-2-yl)propanoate (0.088 g, 99%).

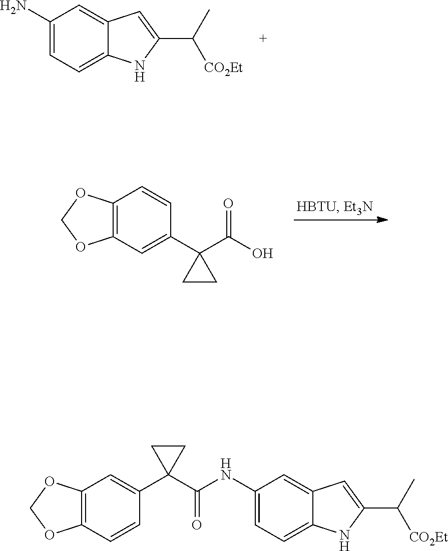

### Ethyl 2-(5-(1-(benzo[d][1,3]dioxol-5-yl)cyclopropanecarboxamido)-1H-indol-2-yl)propanoate

To a solution of 1-(benzo[d][1,3]dioxol-5-yl)cyclopropanecarboxylic acid (0.079 g, 0.384 mmol) in acetonitrile (1.5 mL) were added HBTU (0.146 g, 0.384 mmol) and Et₃N (160 μL, 1.15 mmol) at room temperature. The mixture was allowed to stir at room temperature for 10 min before a solution of ethyl 2-(5-amino-1H-indol-2-yl)propanoate (0.089 g, 0.384 mmol) in acetonitrile (2.16 mL) was added. After addition, the reaction mixture was stirred at room temperature for 2 h. The solvent was evaporated under reduced pressure and the residue was dissolved in dichloromethane. The organic layer was washed with 1N HCl (1×3 mL) and then saturated aqueous NaHCO₃ (1×3 mL). The organic layer was dried over Na₂SO₄, filtered and evaporated under reduced pressure. The crude material was purified by column chromatography on silica gel (ethyl acetate/hexane=1/1) to give ethyl 2-(5-(1-(benzo[d][1,3]dioxol-5-yl)cyclopropanecarboxamido)-1H-indol-2-yl)propanoate (0.081 g, 50%). ¹H NMR (400 MHz, CDCl₃) δ 8.51 (s, 1H), 7.67 (s, 1H), 7.23-7.19 (m, 2H), 7.04-7.01 (m, 3H), 6.89 (d, J=0.0 Hz, 1H), 6.28 (s, 1H), 6.06 (s, 2H), 4.25-4.17 (m, 2H), 3.91 (q, J=7.2 Hz, 1H), 1.72-1.70 (m, 2H), 1.61 (s, 2H), 1.29 (t, J=7.1 Hz, 4H), 1.13-1.11 (m, 2H).

### Example 71

### tert-Butyl 2-(5-(1-(benzo[d][1,3]dioxol-5-yl)cyclopropanecarbox-amido)-1H-indol-2-yl)-2-methylpropylcarbamate

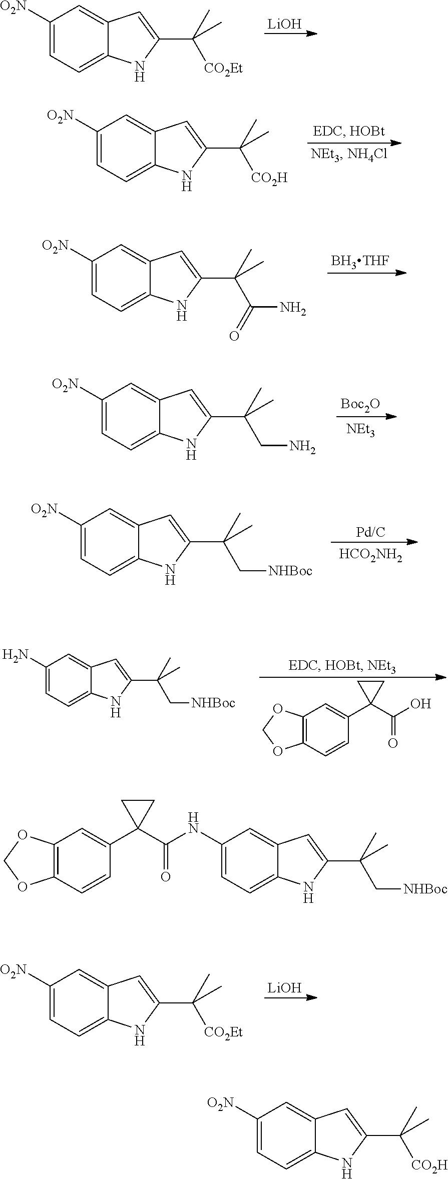

### 2-Methyl-2-(5-nitro-1H-indol-2-yl)propanoic acid

Ethyl 2-methyl-2-(5-nitro-1H-indol-2-yl)propanoate (4.60 g, 16.7 mmol) was dissolved in THF/water (2:1, 30 mL). LiORH₂O (1.40 g, 33.3 mmol) was added and the mixture was stirred at 50° C. for 3 h. The mixture was made acidic by the careful addition of 3N HCl. The product was extracted with ethylacetate and the combined organic phases were washed with brine and dried over magnesium sulfate to give 2-methyl-2-(5-nitro-1H-indol-2-yl)propanoic acid (4.15 g, 99%).

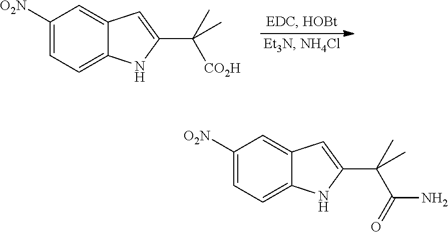

### 2-Methyl-2-(5-nitro-1H-indol-2-yl)propanamide

2-Methyl-2-(5-nitro-1H-indol-2-yl)-propanoic acid (4.12 g, 16.6 mmol) was dissolved in acetonitrile (80 mL). EDC (3.80 g, 0.020 mmol), HOBt (2.70 g, 0.020 mmol), Et₃N (6.9 mL, 0.050 mmol) and ammonium chloride (1.34 g, 0.025 mmol) were added and the mixture was stirred overnight at room temperature. Water was added and the mixture was extracted with ethylacetate. Combined organic phases were washed with brine, dried over magnesium sulfate and dried to give 2-methyl-2-(5-nitro-1H-indol-2-yl)propanamide (4.3 g, 99%).

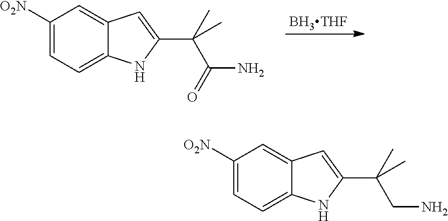 [SMILES: CC(C)(C(N)=O)c1cc2cc([N+](=O)[O-])ccc2[nH]1 ; CC(C)(CN)c1cc2cc([N+](=O)[O-])ccc2[nH]1 ; "BH3·THF"]

### 2-Methyl-2-(5-nitro-1H-indol-2-yl)propan-1-amine

2-Methyl-2-(5-nitro-1H-indol-2-yl) propanamide (200 mg, 0.81 mmol) was suspended in THF (5 ml) and cooled to 0° C. Borane-THF complex solution (1.0 M, 2.4 mL, 2.4 mmol) was added slowly and the mixture was allowed to stir overnight at room temperature. The mixture was cooled to 0° C. and carefully acidified with 3N HCl. THF was evaporated off, water was added and the mixture was washed with ethylacetate. The aqueous layer was made alkaline with 50% NaOH and the mixture was extracted with ethylacetate. The combined organic layers were dried over magnesium sulfate, filtered and evaporated to give 2-methyl-2-(5-nitro-1H-indol-2-yl)propan-1-amine (82 mg, 43%).

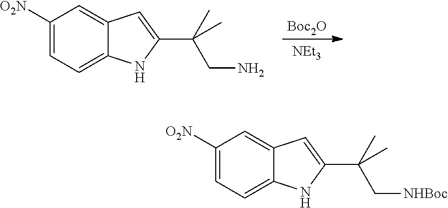 [SMILES: "Boc2O" ; CC(C)(CN)c1cc2cc([N+](=O)[O-])ccc2[nH]1 ; CC(C)(C)OC(=O)NCC(C)(C)c1cc2cc([N+](=O)[O-])ccc2[nH]1 ; "NEt3"]

### tert-Butyl 2-methyl-2-(5-nitro-1H-indol-2-yl)propylcarbamate

2-Methyl-2-(5-nitro-1H-indol-2-yl) propan-1-amine (137 mg, 0.587 mmol) was dissolved in THF (5 mL) and cooled to 0° C. Et₃N (82 μL, 0.59 mmol) and di-tert-butyl dicarbonate (129 mg, 0.587 mmol) were added and the mixture was stirred at room temperature overnight. Water was added and the mixture was extracted with ethylacetate. The residue was purified by silica gel chromatography (10-40% ethylacetate in hexane) to give tert-butyl 2-methyl-2-(5-nitro-1H-indol-2-yl)propylcarbamate (131 mg, 67%).

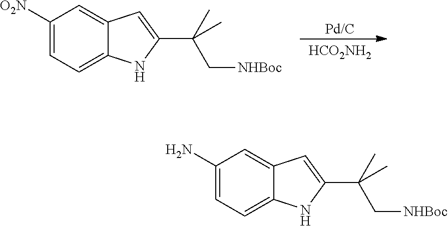 [SMILES: CC(C)(C)OC(=O)NCC(C)(C)c1cc2cc([N+](=O)[O-])ccc2[nH]1 ; "HCO2NH2" ; "Pd/C" ; CC(C)(C)OC(=O)NCC(C)(C)c1cc2cc(N)ccc2[nH]1]

### tert-Butyl 2-(5-amino-1H-indol-2-yl)-2-methylpropylcarbamate

To a solution of tert-butyl 2-methyl-2-(5-nitro-1H-indol-2-yl)propylcarbamate (80 mg, 0.24 mmol) in THF (9 mL) and water (2 mL) was added ammonium formate (60 mg, 0.96 mmol) followed by 10% Pd/C (50 mg). The mixture was stirred at room temperature for 45 minutes. Pd/C was filtered off and the organic solvent was removed by evaporation. The remaining aqueous phase was extracted with dichloromethane. The combined organic phases were dried over magnesium sulfate and evaporated to give tert-butyl 2-(5-amino-1H-indol-2-yl)-2-methylpropylcarbamate (58 mg, 80%).

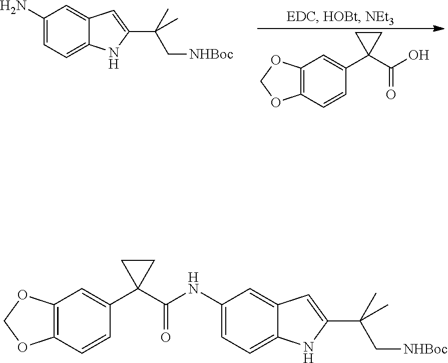

### tert-Butyl 2-(5-(1-(benzo[d][1,3]dioxol-5-yl)cyclopropanecarboxamido)-1H-indol-2-yl)-2-methylpropylcarbamate

tert-Butyl 2-(5-amino-1H-indol-2-yl)-2-methylpropylcarbamate (58 mg, 0.19 mmol), 1-(benzo[d][1,3]dioxol-6-yl)cyclopropanecarboxylic acid (47 mg, 0.23 mmol), EDC (45 mg, 0.23 mmol), HOBt (31 mg, 023 mmol) and Et₃N (80 μL, 0.57 mmol) were dissolved in DMF (4 mL) and stirred overnight at room temperature. The mixture was diluted with water and extracted with ethylacetate. The combined organic phases were dried over magnesium sulfate and evaporated to dryness. The residue was purified by silica gel chromatography (10-30% ethylacetate in hexane) to give tert-butyl 2-(5-(1-(benzo[d][1,3]dioxol-5-yl)cyclopropanecarboxamido)-1H-indol-2-yl)-2-methylpropyl-carbamate (88 mg, 94%). ¹H NMR (400 MHz, CDCl₃) δ 8.32 (s, 1H), 7.62 (d, J=1.5 Hz, 1H), 7.18-7.16 (m, 2H), 7.02-6.94 (m, 3H), 6.85 (d, J=7.8 Hz, 1H), 6.19 (d, J=1.5 Hz, 1H), 6.02 (s, 2H), 4.54 (m, 1H), 3.33 (d, J=6.2 Hz, 2H), 1.68 (dd, J=3.7, 6.8 Hz, 2H), 1.36 (s, 9H), 1.35 (s, 6H), 1.09 (dd, J=3.7, 6.8 Hz, 2H).

### Example 72

### (R)—N-(2-tert-Butyl-1-(2,3-dihydroxypropyl)-1H-indol-5-yl)-1-(2,2-difluorobenzo[d][1,3]dioxol-5-yl)cyclopropanecarboxamide

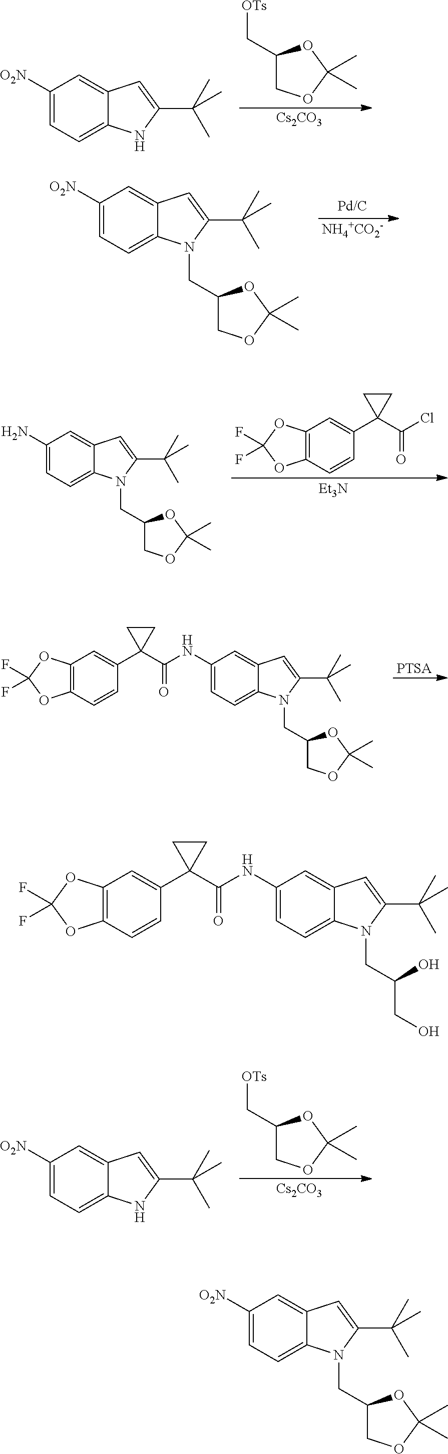

### (R)-2-tert-Butyl-1-((2,2-dimethyl-1,3-dioxolan-4-yl)methyl)-5-nitro-1H-indole

To a stirred solution of (S)-(2,2-dimethyl-1,3-dioxolan-4-yl)methyl 4-methylbenzenesulfonate (1.58 g, 5.50 mmol) in anhydrous DMF (10 mL) under nitrogen gas was added 2-tert-butyl-5-nitro-1H-indole (1.00 g, 4.58 mmol) followed by Cs₂CO₃ (2.99 g, 9.16 mol). The mixture was stirred and heated at 80° C. under nitrogen gas. After 20 hours, 50% conversion was observed by LCMS. The reaction mixture was re-treated with Cs₂CO₃ (2.99 g, 9.16 mol) and (S)-(2,2-dimethyl-1,3-dioxolan-4-yl)methyl 4-methylbenzenesulfonate (1.58 g, 5.50 mmol) and heated at 80° C. for 24 hours. The reaction mixture was cooled to room temperature. The solids were filtered and washed with ethyl acetate and hexane (1:1). The layers were separated and the organic layer was washed with water (2×10 mL) and brine (2×10 mL). The organic layer was dried over Na₂SO₄, filtered and evaporated under reduced pressure. The residue was purified by column chromatography on silica gel (dichloromethane/hexane=1.5/1) to give (R)-2-tert-butyl-1-((2,2-dimethyl-1,3-dioxolan-4-yl)methyl)-5-nitro-1H-indole (1.0 g, 66%). ¹H NMR (400 MHz, CDCl₃) δ 8.48 (d, J=2.2 Hz, 1H), 8.08 (dd, J=2.2, 9.1 Hz, 1H), 7.49 (d, J=9.1 Hz, 1H), 6.00 (s, 1H), 4.52-4.45 (m, 3H), 4.12 (dd, J=6.0, 8.6 Hz, 1H), 3.78 (dd, J=6.0, 8.6 Hz, 1H), 1.53 (s, 3H), 1.51 (s, 9H), 1.33 (s, 3H).

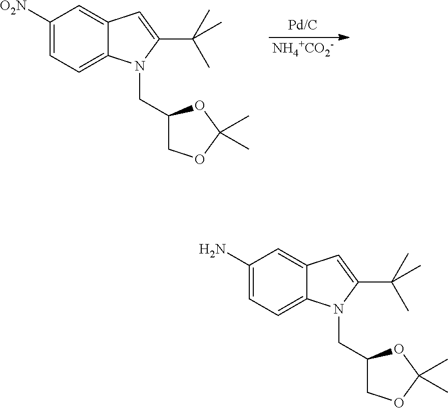

### (R)-2-tert-Butyl-14(2,2-dimethyl-1,3-dioxolan-4-yl)methyl-1H-indol-5-amine

To a stirred solution of (R)-2-tert-butyl-1-((2,2-dimethyl-1,3-dioxolan-4-yl)methyl)-5-nitro-1H-indole (1.0 g, 3.0 mmol) in ethanol (20 mL) and water (5 mL) was added ammonium formate (0.76 g, 12 mmol) followed by slow addition of 10% palladium on carbon (0.4 g). The mixture was stirred at room temperature for 1 h. The reaction mixture was filtered through a plug of celite and rinsed with ethyl acetate. The filtrate was evaporated under reduced pressure and the crude product was dissolved in ethyl acetate. The organic layer was washed with water (2×5 mL) and brine (2×5 mL). The organic layer was dried over Na₂SO₄, filtered and evaporated under reduced pressure to give (R)-2-tert-butyl-1-((2,2-dimethyl-1,3-dioxolan-4-yl)methyl-1H-indol-5-amine (0.89 g, 98%). ¹H NMR (400 MHz, CDCl₃) δ 7.04 (d, J=4 Hz, 1H), 6.70 (d, J=2.2 Hz, 1H), 6.48 (dd, J=2.2, 8.6 Hz, 1H), 6.05 (s, 1H,), 4.38-4.1 (m, 2H), 4.21 (dd, J=7.5, 16.5 Hz, 1H), 3.87 (dd, J=6.0, 8.6 Hz, 1H), 3.66 (dd, J=6.0, 8.6 Hz, 1H), 3.33 (br s, 2H), 1.40 (s, 3H), 1.34 (s, 9H), 1.25 (s, 3H).

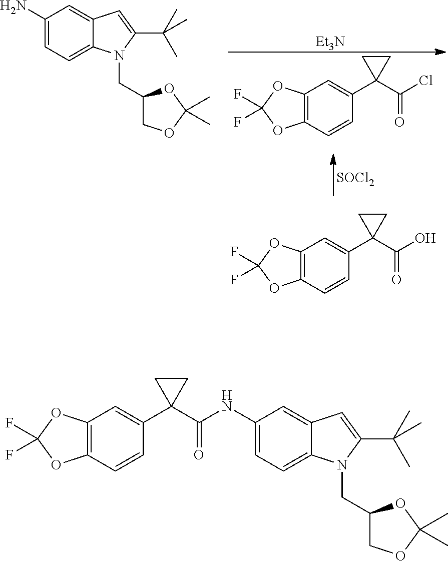

### N—((R)-2-tert-Butyl-1-((2,2-dimethyl-1,3-dioxolan-4-yl)methyl)-1H-indol-5-yl)-1-(2,2-difluorobenzo[d][1,3]dioxol-5-yl)cyclopropanecarboxamide

To 1-(benzo[d][1,3]dioxol-5-yl)cyclopropanecarboxylic acid (0.73 g, 3.0 mmol) was added thionyl chloride (660 μL, 9.0 mmol) and DMF (20 μL) at room temperature. The mixture was stirred for 30 minutes before the excess thionyl chloride was evaporated under reduced pressure. To the resulting acid chloride, dichloromethane (6.0 mL) and Et₃N (2.1 mL, 15 mmol) were added. A solution of (R)-2-tert-butyl-1-(2,2-dimethyl-1,3-dioxolan-4-yl)methyl-1H-indol-5-amine (3.0 mmol) in dichloromethane (3.0 mL) was added to the cooled acid chloride solution. After addition, the reaction mixture was stirred at room temperature for 45 minutes. The reaction mixture was filtered and the filtrate was evaporated under reduced pressure. The residue was purified by column chromatography on silica gel (ethyl acetate/hexane=3/7) to give N4R)-2-tert-butyl-14(2,2-dimethyl-1,3-dioxolan-4-yl)methyl)-1H-indol-5-yl)-1-(2,2-difluorobenzo[d][1,3]dioxol-5-yl)cyclopropanecarboxamide (1.33 g, 84%). ¹H NMR (400 MHz, CDCl₃) δ 7.48 (d, J=2 Hz, 1H,), 7.31 (dd, J=2, 8 Hz, 1H), 7.27 (dd, J=2, 8 Hz, 1H), 7.23 (d, J=8 Hz, 1H), 7.14 (d, J=8 Hz, 1H), 7.02 (dd, J=2, 8 Hz, 1H), 6.92 (br s, 1H), 6.22 (s, 1H), 4.38-4.05 (m, 3H), 3.91 (dd, J=5, 8 Hz, 1H), 3.75 (dd, J=5, 8 Hz, 1H), 2.33 (q, J=8 Hz, 2H), 1.42 (s, 3H), 1.37 (s, 9H), 1.22 (s, 3H), 1.10 (q, J=8 Hz, 2H).

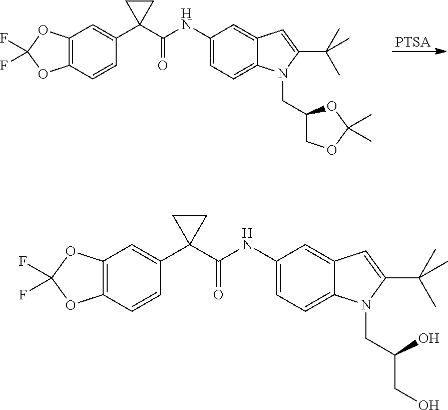

### N—((R)-2-tert-Butyl-1-((2,3-dihydroxypropyl)-1H-indol-5-yl)-1-(2,2-difluorobenzo-[d][1,3]dioxol-5-yl)cyclopropanecarboxamide

To a stirred solution of N-(2-tert-butyl-1-((2,2-dimethyl-1,3-dioxolan-4-yl)methyl)-1H-indol-5-yl)-1-(2,2-difluorobenzo[d][1,3]dioxol-5-yl)cyclopropanecarboxamide (1.28 g, 2.43 mmol) in methanol (34 mL) and water (3.7 mL) was added para-toluenesulfonic acid-hydrate (1.87 g, 9.83 mmol). The reaction mixture was stirred and heated at 80° C. for 25 minutes. The solvent was evaporated under reduced pressure. The crude product was dissolved in ethyl acetate. The organic layer was washed with saturated aqueous NaHCO₃ (2×10 mL) and brine (2×10 mL). The organic layer was dried over Na₂SO₄, filtered and evaporated under reduced pressure. The residue was purified by column chromatography on silica gel (ethyl acetate/hexane=13/7) to give N—((R)-2-tert-butyl-1-((2,3-dihydroxypropyl)-1H-indol-5-yl)-1-(2,2-difluorobenzo[d][1,3]dioxol-5-yl)cyclopropanecarboxamide (0.96 g, 81%). ¹H NMR (400 MHz, CDCl₃) δ 7.50 (d, J=2 Hz, 1H), 7.31 (dd, J=2, 8 Hz, 1H), 7.27 (dd, J=2, 8 Hz, 1H), 7.23 (d, J=8 Hz, 1H), 7.14 (d, J=8 Hz, 1H), 7.02 (br s, 1H,), 6.96 (dd, J=2, 8 Hz, 1H), 6.23 (s, 1H), 4.35 (dd, J=8, 15 Hz, 1H), 4.26 (dd, J=4, 15 Hz, 1H,), 4.02-3.95 (m, 1H), 3.60 (dd, J=4, 11 Hz, 1H), 3.50 (dd, J=5, 11 Hz, 1H), 1.75 (q, J=8 Hz, 3H), 1.43 (s, 9H), 1.14 (q, J=8 Hz, 3H).

### Example 73

### 3-(2-tert-Butyl-5-(1-(2,2-difluorobenzo[d][1,3]dioxol-5-yl)cyclopropanecarboxamido)-1H-indol-1-yl)-2-hydroxypropanoic acid

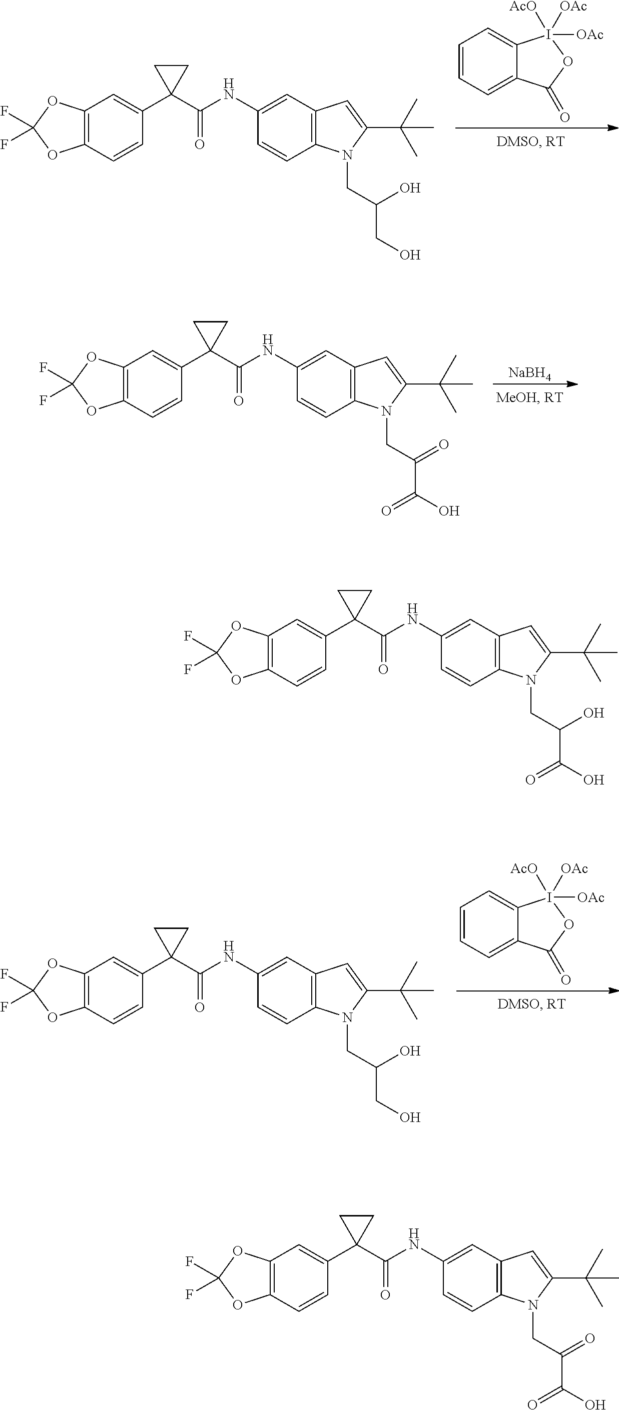

### 3-(2-tert-Butyl-5-(1-(2,2-difluorobenzo[d][1,3]dioxol-5-yl)cyclopropanecarbox-amido)-1H-indol-1-yl)-2-oxopropanoic acid

To a solution of N-(2-tert-butyl-1-(2,3-dihydroxypropyl)-1H-indol-5-yl)-1-(2,2-difluorobenzo[d][1,3]dioxol-5-yl)cyclopropane-carboxamide (97 mg, 0.20 mmol) in DMSO (1 mL) was added Dess-Martin periodinane (130 mg, 0.30 mmol). The mixture was stirred at room temperature for 3 h. The solid was filtered off and washed with EtOAc. The filtrate was partitioned between EtOAc and water. The aqueous layer was extracted with EtOAc twice and the combined organic layers were washed with brine and dried over MgSO₄. After the removal of solvent, the residue was purified by preparative TLC to yield 3-(2-tert-butyl-5-(1-(2,2-difluorobenzo[d][1,3]dioxol-5-yl)cyclopropanecarboxamido)-1H-indol-1-yl)-2-oxopropanoic acid that was used without fluffier purification.

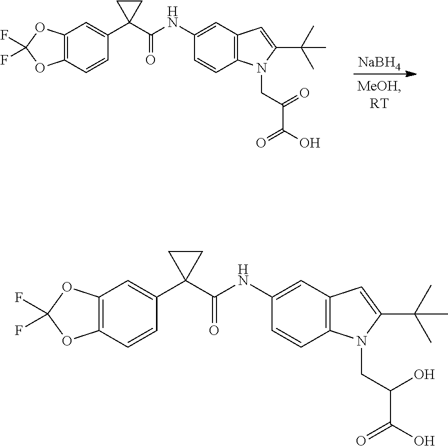

### 3-(2-tert-Butyl-5-(1-(2,2-difluorobenzo[d][1,3]dioxol-5-yl)cyclopropanecarbox-amido)-1H-indol-1-yl)-2-hydroxypropanoic acid

To a solution of 3-(2-tert-butyl-5-(1-(2,2-difluorobenzo[d][1,3]dioxol-5-yl)cyclopropanecarboxamido)-1H-indol-1-yl)-2-oxopropanoic acid (50 mg, 0.10 mmol) in MeOH (1 mL) was added NaBH₄ (19 mg, 0.50 mmol) at 0° C. The mixture was stirred at room temperature for 15 min. The resulting mixture was partitioned between EtOAc and water. The aqueous layer was extracted with EtOAc twice and the combined organic layers were washed with brine and dried over anhydrous MgSO₄. After the removal of the solvent, the residue was taken up in DMSO and purified by preparative LC/MS to give 3-(2-tert-butyl-5-(1-(2,2-difluorobenzo[d][1,3]dioxol-5-yl)cyclopropanecarboxamido)-1H-indol-1-yl)-2-hydroxypropanoic acid. ¹H NMR (400 MHz, CDCl₃) δ 7.36 (s), 7.27-7.23 (m, 2H), 7.15-7.11 (m, 2H), 6.94 (d, J=8.5 Hz, 1H), 6.23 (s, 1H), 4.71 (s, 3H), 4.59 (q, J=10.3 Hz, 1H), 4.40-4.33 (m, 2H), 1.70 (d, J=1.9 Hz, 2H), 1.15 (q, J=4.0 Hz, 2H). ¹³C NMR (400 MHz, CDCl₃) δ 173.6, 173.1, 150.7, 144.1, 143.6, 136.2, 135.4, 134.3, 131.7, 129.2, 129.0, 127.6, 126.7, 116.6, 114.2, 112.4, 110.4, 110.1, 99.7, 70.3, 48.5, 32.6, 30.9, 30.7, 16.8. MS (ESI) m/e (M+H⁺) 501.2.

### Example 74

### (R)—N-(2-tert-Butyl-1-(2,3-dihydroxypropyl)-1H-indol-5-yl)-1-(2,2-dideuteriumbenzo[d][1,3]dioxol-5-yl)cyclopropanecarboxamide

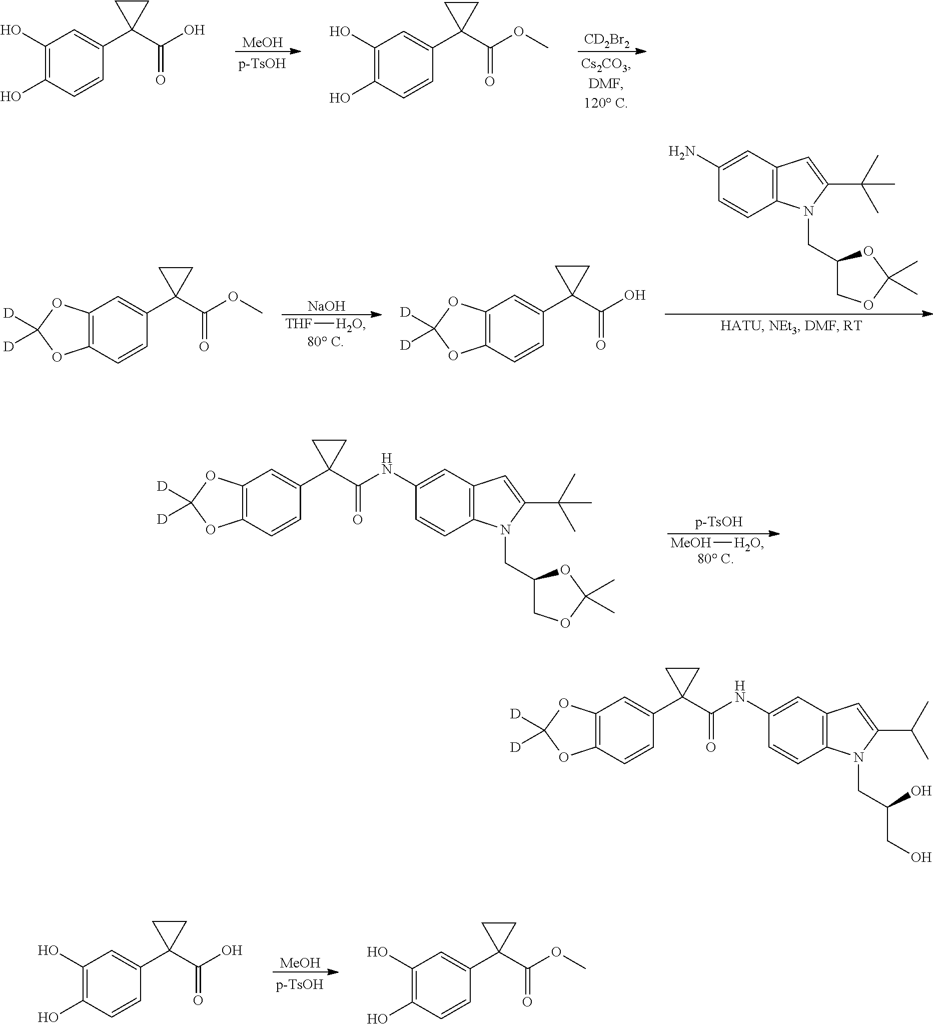

### Methyl 1-(3,4-dihydroxyphenyl)cyclopropanecarboxylate

To a solution of 1-(3,4-dihydroxyphenyl)cyclopropanecarboxylic acid (190 mg, 1.0 mmol) in MeOH (3 mL) was added 4-methylbenzenesulfonic acid (19 mg, 0.10 mmol). The mixture was heated at 80° C. overnight. The reaction mixture was concentrated in vacuo and partitioned between EtOAc and water. The aqueous layer was extracted with EtOAc twice and the combined organic layers were washed with sat. NaHCO₃ and brine and dried over MgSO₄. After the removal of solvent, the residue was dried in vacuo to yield methyl 1-(3,4-dihydroxyphenyl)cyclopropanecarboxylate (190 mg, 91%) that was used without further purification. ¹H NMR (400 MHz, DMSO-d⁶) S 6.76-6.71 (m, 2H), 6.66 (d, J=7.9 Hz, 1H), 3.56 (s, 3H), 1.50 (q, J=3.6 Hz, 2H), 1.08 (q, J=3.6 Hz, 2H).

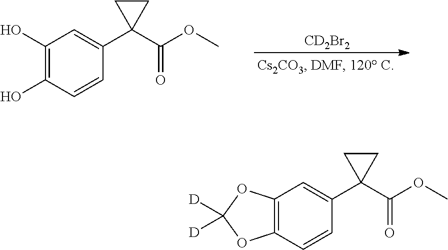 [SMILES: COC(=O)C1(c2ccc(O)c(O)c2)CC1 ; "CD2Br2" ; [2H]C1([2H])Oc2ccc(C3(C(=O)OC)CC3)cc2O1]

### Methyl 1-(2,2-dideuteriumbenzo[d][1,3]dioxol-5-yl)cyclopropanecarboxylate

To a solution of methyl 1-(3,4-dihydroxyphenyl)cyclopropanecarboxylate (21 mg, 0.10 mmol) and CD₂Br₂ (35 mg, 0.20 mmol) in DMF (0.5 mL) was added Cs₂CO₃ (19 mg, 0.10 mmol). The mixture was heated at 120° C. for 30 min. The reaction mixture was partitioned between EtOAc and water. The aqueous layer was extracted with EtOAc twice and the combined organic layers were washed with 1N NaOH and brine before being dried over MgSO₄. After the removal of solvent, the residue was dried in vacuo to yield methyl 1-(2,2-dideuteriumbenzo[d][1,3]dioxol-5-yl)cyclopropanecarboxylate (22 mg) that was used without further purification. ¹H NMR (400 MHz, CDCl₃) δ 6.76-6.71 (m, 2H), 6.66 (d, J=7.9 Hz, 1H), 3.56 (s, 3H), 1.50 (q, J=3.6 Hz, 2H), 1.08 (q, J=3.6 Hz, 2H).

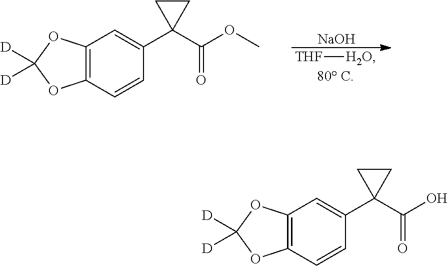

### 1-(2,2-Dideuteriumbenzo[d][1,3]dioxol-5-yl)cyclopropanecarboxylic acid

To a solution of methyl 1-(2,2-dideuteriumbenzo[d][1,3]dioxol-5-yl)cyclopropanecarboxylate (22 mg, 0.10 mmol) in THF (0.5 mL) was added NaOH (1N, 0.25 mL, 0.25 mmol). The mixture was heated at 80° C. for 2 h. The reaction mixture was partitioned between EtOAc and 1N NaOH. The aqueous layer was extracted with EtOAc twice, neutralized with 1N HCl and extracted with EtOAc twice. The combined organic layers were washed with brine and dried over MgSO₄. After the removal of solvent, the residue was dried in vacuo to yield 1-(2,2-dideuteriumbenzo[d][1,3]dioxol-5-yl)cyclopropanecarboxylic acid (21 mg) that was used without further purification.

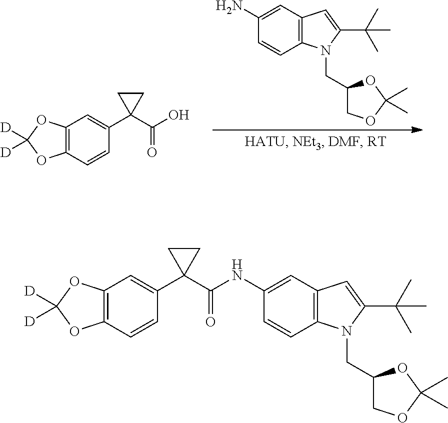 [SMILES: [2H]C1([2H])Oc2ccc(C3(C(=O)O)CC3)cc2O1 ; CC1(C)OC[C@@H](Cn2c(C(C)(C)C)cc3cc(N)ccc32)O1 ; [2H]C1([2H])Oc2ccc(C3(C(=O)Nc4ccc5c(c4)cc(C(C)(C)C)n5C[C@@H]4COC(C)(C)O4)CC3)cc2O1]

### (R)—N-(2-tert-Butyl-14(2,2-dimethyl-1,3-dioxolan-4-yl)methyl)-1H-indol-5-yl)-1-(2,2-dideuteriumbenzo[d][1,3]dioxol-5-yl)cyclopropanecarboxamide

To a solution of 1-(2,2-dideuteriumbenzo[d][1,3]dioxol-5-yl)cyclopropanecarboxylic acid (21 mg, 0.10 mmol), (R)-2-tert-butyl-1-((2,2-dimethyl-1,3-dioxolan-4-yl)methyl)-1H-indol-5-amine (30 mg, 0.10 mmol), HATU (42 mg, 0.11 mol) in DMF (1 mL) was added triethylamine (0.030 mL, 0.22 mmol). The mixture was heated at room temperature for 5 min. The reaction mixture was partitioned between EtOAc and water. The aqueous layer was extracted with EtOAc twice and the combined organic layers were washed with 1N NaOH, 1N HCl, and brine before being dried over MgSO₄. After the removal of solvent, the residue was purified by column chromatography (20-40% ethyl acetate/hexane) to yield (R)—N-(2-tert-butyl-1-((2,2-dimethyl-1,3-dioxolan-4-yl)methyl)-1H-indol-5-yl)-1-(2,2-dideuteriumbenzo[d][1,3]dioxol-5-yl)cyclopropanecarboxamide (24 mg, 49% from methyl 1-(3,4-dihydroxyphenyl)cyclopropanecarboxylate). MS (ESI) m/e (M+H₊) 493.5.

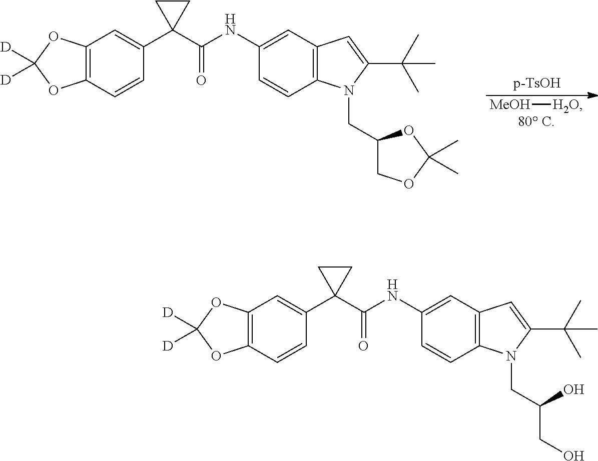

### (R)—N-(2-tert-Butyl-1-(2,3-dihydroxypropyl)-1H-indol-5-yl)-1-(2,2-dideuterium-benzo[d][1,3]dioxol-5-yl)cyclopropanecarboxamide

To a solution of (R)—N-(2-tert-butyl-1-((2,2-dimethyl-1,3-dioxolan-4-yl)methyl)-1H-indol-5-yl)-1-(2,2-dideuterium-benzo[d][1,3]dioxol-5-yl)cyclopropanecarboxamide (24 mg, 0.050 mmol), in methanol (0.5 mL) and water (0.05 mL) was added 4-methylbenzenesulfonic acid (2.0 mg, 0.010 mmol). The mixture was heated at 80° C. for 30 min. The reaction mixture was partitioned between EtOAc and water. The aqueous layer was extracted with EtOAc twice and the combined organic layers were washed with sat. NaHCO₃ and brine before being dried over MgSO₄. After the removal of solvent, the residue was purified by preparative HPLC to yield (R)—N-(2-tert-butyl-14(2,2-dimethyl-1,3-dioxolan-4-yl)methyl)-1H-indol-5-yl)-1-(2,2-dideuteriumbenzo[d][1,3]dioxol-5-yl)cyclopropanecarboxamide (12 mg, 52%). ¹H NMR (400 MHz, CDCl₃) δ 7.44 (d, J=2.0 Hz, 1H), 7.14 (dd, J=22.8, 14.0 Hz, 2H), 6.95-6.89 (m, 2H), 6.78 (d, J=7.8 Hz, 1H), 6.14 (s, 1H), 4.28 (dd, J=15.1, 8.3 Hz, 1H), 4.19 (dd, J=15.1, 4.5 Hz, 1H), 4.05 (q, J=7.1 Hz, 1H), 3.55 (dd, J=11.3, 4.0 Hz, 1H), 3.45 (dd, J=11.3, 5.4 Hz, 1H), 1.60 (q, J=3.5 Hz, 2H), 1.35 (s, 9H), 1.02 (q, J=3.5 Hz, 2H). ¹³C NMR (400 MHz, CDCl₃) δ 171.4, 149.3, 147.1, 146.5, 134.8, 132.3, 129.2, 126.5, 123.6, 114.3, 111.4, 110.4, 109.0, 107.8, 98.5, 70.4, 63.1, 46.6, 31.6, 30.0, 29.8, 15.3. MS (ESI) m/e (M+H⁺) 453.5.

It is further noted that the mono-deuterated analogue for this compound can be synthesized by substitution the reagent CHDBR₂ for CD₂BR₂ and following the procedures described in example 74. Furthermore, deuterated analogues of the compounds as described herein such as of Formula D can be produced using known synthetic methods as well as the methodology described herein. The deuterated analogues include both di and mono-deuterated analogues of the compounds of the present invention. The di and mono deuterated analogues of the compounds exhibit measurable acitivity when tested using the assays described below.

### Example 75

### 4-(5-(1-(Benzo[d][1,3]dioxol-5-yl)cyclopropanecarboxamido)-1H-indol-2-yl)-4-methylpentanoic acid

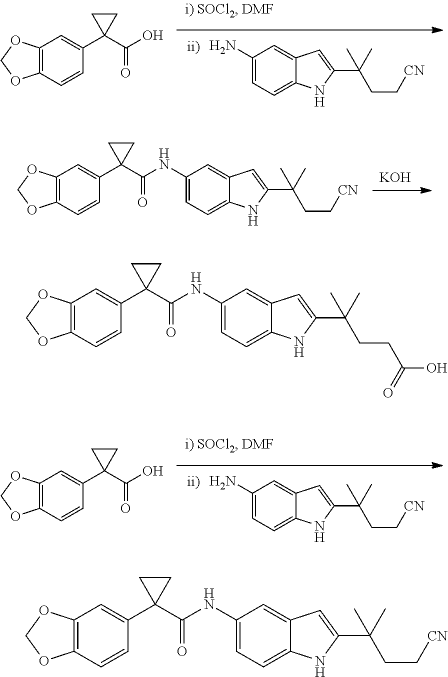

### 1-(Benzo[d][1,3]dioxol-5-yl)-N-(2-(4-cyano-2-methylbutan-2-yl)-1H-indol-5-yl)cyclopropanecarboxamide

To 1-(benzo[d][1,3]dioxol-5-yl)cyclopropanecarboxylic acid (0.068 g, 0.33 mmol) was added thionyl chloride (72 μL, 0.99 mmol) and DMF (20 μL) at room temperature. The mixture was stirred for 30 minutes before the excess thionyl chloride was evaporated under reduced pressure. To the resulting acid chloride, dichloromethane (0.5 mL) and Et₃N (230 μL, 1.7 mmol) were added. A solution of 4-(5-amino-1H-indol-2-yl)-4-methylpentanenitrile (0.33 mmol) in dichloromethane (0.5 mL) was added to the acid chloride solution and the mixture was stirred at room temperature for 1.5 h. The resulting mixture was diluted with dichloromethane and washed with 1N HCl (2×2 mL), saturated aqueous NaHCO₃ (2×2 mL) and brine (2×2 mL). The organic layer was dried over anhydrous Na₂SO₄ and evaporated under reduced pressure to give 1-(benzo[d][1,3]dioxol-5-yl)-N-(2-(4-cyano-2-methylbutan-2-yl)-1H-indol-5-yl)cyclopropanecarboxamide.

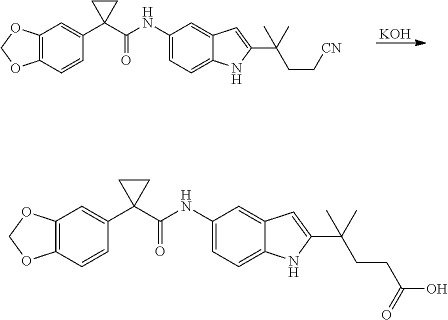 [SMILES: CC(C)(CCC#N)c1cc2cc(NC(=O)C3(c4ccc5c(c4)OCO5)CC3)ccc2[nH]1 ; "KOH" ; CC(C)(CCC(=O)O)c1cc2cc(NC(=O)C3(c4ccc5c(c4)OCO5)CC3)ccc2[nH]1]

### 4-(5-(1-(Benzo[d][1,3]dioxol-5-yl)cyclopropanecarboxamido)-1H-indol-2-yl)-4-methylpentanoic acid

A mixture of 1-(benzo[d][1,3]dioxol-5-yl)-N-(2-(4-cyano-2-methylbutan-2-yl)-1H-indol-5-yl)cyclopropanecarboxamide (0.060 g, 0.15 mmol) and KOH (0.081 g, 1.5 mmol) in 50% EtOH/water (2 mL) was heated in the microwave at 100° C. for 1 h. The solvent was evaporated under reduced pressure. The crude product was dissolved in DMSO (1 mL), filtered, and purified by reverse phase preparative HPLC to give 4-(5-(1-(benzo[d][1,3]dioxol-5-yl)cyclopropanecarboxamido)-1H-indol-2-yl)-4-methylpentanoic acid. ¹H NMR (400 MHz, DMSO-d6) δ 11.98 (s, 1H), 10.79 (s, 1H), 8.44 (s, 1H), 7.56 (s, 1H), 7.15 (d, J=8.6 Hz, 1H), 7.03-6.90 (m, 4H), 6.05 (s, 1H), 6.02 (s, 2H), 1.97-1.87 (m, 4H), 1.41-1.38 (m, 2H), 1.30 (s, 6H), 1.04-1.02 (m, 2H).

### Example 76

### 1-(Benzo[d][1,3]dioxol-5-yl)-N-(2-(1-hydroxypropan-2-yl)-1H-indol-5-yl)cyclopropanecarboxamide

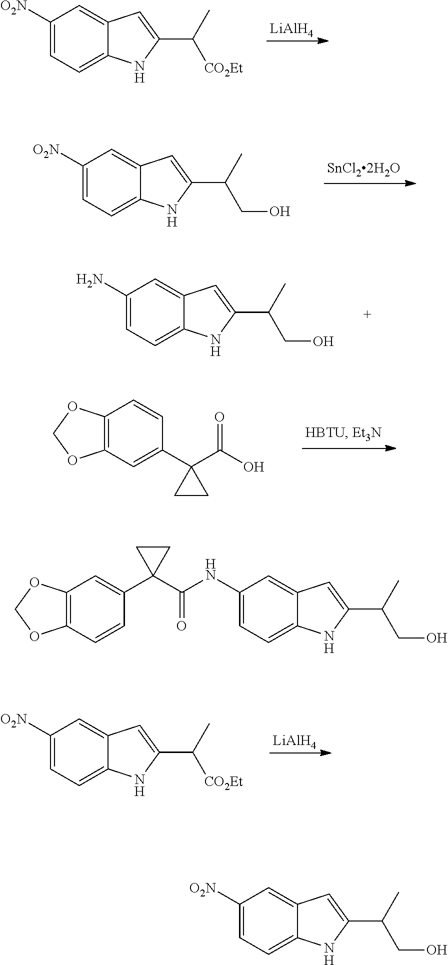

### 2-(5-Nitro-1H-indol-2-yl)propan-1-ol

To a cooled solution of LiAlH₄ (1.0 M in THF, 1.2 mL, 1.2 mmol) in THF (5.3 mL) at 0° C. was added a solution of ethyl 2-(5-nitro-1H-indol-2-yl)propanoate (0.20 g, 0.76 mmol) in THF (3.66 mL) dropwise. After addition, the mixture was allowed to warm up to room temperature and was stirred at room temperature for 3 h. The mixture was cooled to 0° C. Water (2 mL) was slowly added followed by careful addition of 15% NaOH (2 mL) and water (4 mL). The mixture was stirred at room temperature for 0.5 h and was then filtered through a short plug of celite using ethyl acetate. The organic layer was separated from the aqueous layer, dried over Na₂SO₄, filtered and evaporated under reduced pressure. The residue was purified by column chromatography on silica gel (ethyl acetate/hexane=1/1) to give 2-(5-nitro-1H-indol-2-yl)propan-1-ol (0.14 g, 81%).

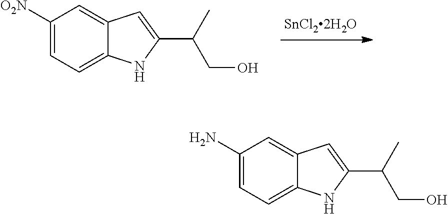 [SMILES: CC(CO)c1cc2cc([N+](=O)[O-])ccc2[nH]1 ; CC(CO)c1cc2cc(N)ccc2[nH]1]

### 2-(5-Amino-1H-indol-2-yl)propan-1-ol

To a solution of 2-(5-nitro-1H-indol-2-yl)propan-1-ol (0.13 g, 0.60 mmol) in ethanol (5 mL) was added tin chloride dihydrate (0.67 g, 3.0 mmol). The mixture was heated in the microwave at 120° C. for 1 h. The mixture was diluted with ethyl acetate before water and saturated aqueous NaHCO₃ were added. The reaction mixture was filtered through a plug of celite using ethyl acetate. The organic layer was separated from the aqueous layer, dried over Na₂SO₄, filtered and evaporated under reduced pressure to give 2-(5-amino-1H-indol-2-yl)propan-1-ol (0.093 g, 82%).

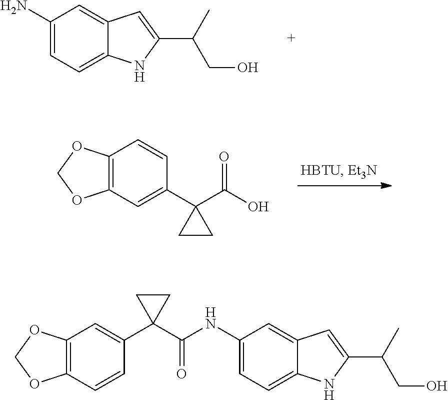 [SMILES: CC(CO)c1cc2cc(N)ccc2[nH]1 ; O=C(O)C1(c2ccc3c(c2)OCO3)CC1 ; CC(CO)c1cc2cc(NC(=O)C3(c4ccc5c(c4)OCO5)CC3)ccc2[nH]1]

### 1-(Benzo[d][1,3]dioxol-5-yl)-N-(2-(1-hydroxypropan-2-yl)-1H-indol-5-yl)cyclopropanecarboxamide

To a solution of 1-(benzo[d][1,3]dioxol-5-yl)cyclopropanecarboxylic acid (0.10 g, 0.49 mmol) in acetonitrile (2.0 mL) were added HBTU (0.185 g, 0.49 mmol) and Et₃N (205 μL, 1.47 mmol) at room temperature. The mixture was allowed to stir at room temperature for 10 minutes before a slurry of 2-(5-amino-1H-indol-2-yl)propan-1-ol (0.093 g, 0.49 mmol) in acetonitrile (2.7 mL) was added. After addition, the reaction mixture was stirred at room temperature for 5.5 h. The solvent was evaporated under reduced pressure and the residue was dissolved in dichloromethane. The organic layer was washed with 1N HCl (1×3 mL) and saturated aqueous NaHCO₃ (1×3 mL). The organic layer was dried over Na₂SO₄, filtered and evaporated under reduced pressure. The crude material was purified by column chromatography on silica gel (ethyl acetate/hexane=13/7) to give 1-(benzo[d][1,3]dioxol-5-yl)-N-(2-(1-hydroxypropan-2-yl)-1H-indol-5-yl)cyclopropanecarboxamide (0.095 g, 51%). ¹H NMR (400 MHz, DMSO-d6) δ 10.74 (s, 1H), 8.38 (s, 1H), 7.55 (s, 1H), 7.14 (d, J=8.6 Hz, 1H), 7.02-6.90 (m, 4H), 6.06 (s, 1H), 6.02 (s, 2H), 4.76 (t, J=5.3 Hz, 1H), 3.68-3.63 (m, 1H), 3.50-3.44 (m, 1H), 2.99-2.90 (m, 1H), 1.41-1.38 (m, 2H), 1.26 (d, J=7.0 Hz, 3H), 1.05-1.02 (m, 2H).

### Example 77

### 1-(Benzo[d][1,3]dioxol-5-yl)-N-(2-tert-butyl-1H-indol-5-yl)-N-methylcyclopropanecarboxamide

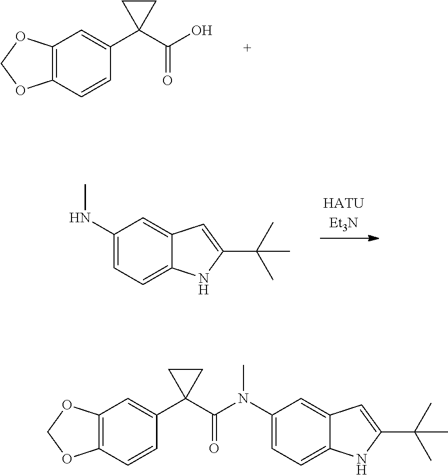 [SMILES: O=C(O)C1(c2ccc3c(c2)OCO3)CC1 ; CNc1ccc2[nH]c(C(C)(C)C)cc2c1 ; CN(C(=O)C1(c2ccc3c(c2)OCO3)CC1)c1ccc2[nH]c(C(C)(C)C)cc2c1]

### 1-(Benzo[d][1,3]dioxol-5-yl)-N-(2-tert-butyl-1H-indol-5-yl)-N-methylcyclopropanecarboxamide

2-tert-Butyl-N-methyl-1H-indol-5-amine (20.2 mg, 0.100 mmol) and 1-(benzo[d][1,3]dioxol-5-yl)cyclopropanecarboxylic acid (20.6 mg, 0.100 mmol) were dissolved in N,N-dimethylformamide (1 mL) containing triethylamine (42.1 μL, 0.300 mmol) and a magnetic stir bar. O-(7-Azabenzotriazol-1-yl)-N,N,N′,N′-tetramethyluronium hexafluorophosphate (42 mg, 0.11 mmol) was added to the mixture and the resulting solution was allowed to stir for 16 h at 80° C. The crude product was then purified by preparative HPLC utilizing a gradient of 0-99%. acetonitrile in water containing 0.05% trifluoroacetic acid to yield 1-(benzo[d][1,3]dioxol-5-yl)-N-(2-tert-butyl-1H-indol-5-yl)-N-methylcyclopropanecarboxamide. ESI-MS m/z calc. 390.2. found 391.3 (M+1)⁺. Retention time of 3.41 minutes.

### Example 78

### N-(2-tert-Butyl-1-methyl-1H-indol-5-yl)-1-(benzo[d][1,3]dioxol-6-yl)-N-methylcyclopropanecarboxamide

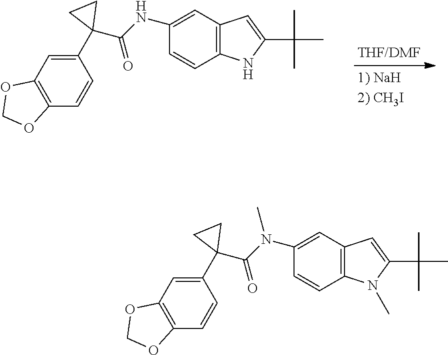 [SMILES: CC(C)(C)c1cc2cc(NC(=O)C3(c4ccc5c(c4)OCO5)CC3)ccc2[nH]1 ; CN(C(=O)C1(c2ccc3c(c2)OCO3)CC1)c1ccc2c(c1)cc(C(C)(C)C)n2C]

Sodium hydride (0.028 g, 0.70 mmol, 60% by weight dispersion in oil) was slowly added to a stirred solution of N-(2-tert-butyl-1H-indol-5-yl)-1-(benzo[d][1,3]dioxol-6-yl)cyclopropanecarboxamide (0.250 g, 0.664 mmol) in a mixture of 4.5 mL of anhydrous tetrahydrofuran (THF) and 0.5 mL of anhydrous N,N-dimethylformamide (DMF). The resulting suspension was allowed to stir for 2 minutes and then iodomethane (0.062 mL, 1.0 mmol) was added to the reaction mixture. Two additional aliquots of sodium hydride and iodomethane were required to consume all of the starting material which was monitored by LC/MS. The crude reaction product was evaporated to dryness, redissolved in a minimum of DMF and purified by preparative LC/MS chromatography to yield the pure product (0.0343 g, 13%) ESI-MS m/z calc. 404.2. found 405.3 (M+1)⁺. Retention time of 3.65 minutes.

### Example 79

### 1-(Benzo[d][1,3]dioxol-5-yl)-N-(2-(hydroxymethyl)-1H-indol-5-yl)cyclopropanecarboxamide

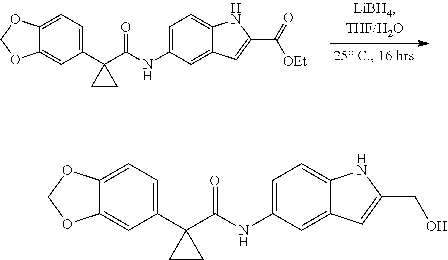 [SMILES: CCOC(=O)c1cc2cc(NC(=O)C3(c4ccc5c(c4)OCO5)CC3)ccc2[nH]1 ; O=C(Nc1ccc2[nH]c(CO)cc2c1)C1(c2ccc3c(c2)OCO3)CC1]

Ethyl 5-(1-(benzo[d][1,3]dioxol-5-yl)cyclopropanecarboxamido)-1H-indole-2-carboxylate (1.18 g, 3.0 mmol) was added to a solution of LiBH₄ (132 mg, 6.0 mmol) in THF (10 mL) and water (0.1 mL). The mixture was allowed to stir for 16 h at 25° C. before it was quenched with water (10 mL) and slowly made acidic by addition of 1N HCl. The mixture was extracted with three 50-mL portions of ethyl acetate. The organic extracts were dried over Na₂SO₄ and evaporated to yield 1-(benzo[d][1,3]dioxol-5-yl)-N-(2-(hydroxymethyl)-1H-indol-5-yl)cyclopropanecarboxamide (770 mg, 73%). A small amount was further purified by reverse phase HPLC. ESI-MS m/z calc. 350.4. found 351.3 (M+1)⁺; retention time 2.59 minutes.

### Example 80

### 5-(1-(Benzo[d][1,3]dioxol-5-yl)cyclopropanecarboxamido)-N-tert-butyl-1H-indole-2-carboxamide

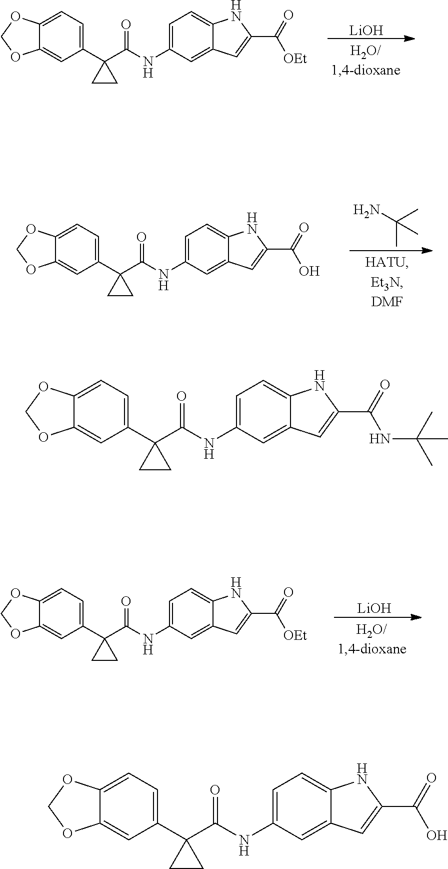

### 5-O-(Benzo[d][1,3]dioxol-5-yl)cyclopropanecarboxamido)-1H-indole-2-carboxylic acid

Ethyl 5-(1-(benzo[d][1,3]dioxol-5-yl)cyclopropanecarboxamido)-1H-indole-2-carboxylate (392 mg, 1.0 mmol) and LiOH (126 mg, 3 mmol) were dissolved in H₂O (5 mL) and 1,4-dioxane (3 mL). The mixture was heated in an oil bath at 100° C. for 24 hours before it was cooled to room temperature. The mixture was acidified with 1N HCl and it was extracted with three 20 mL portions of dichloromethane. The organic extracts were dried over Na₂SO₄ and evaporated to yield 5-(1-(benzo[d][1,3]-dioxol-5-yl)cyclopropanecarboxamido)-1H-indole-2-carboxylic acid (302 mg, 83%). A small amount was further purified by reverse phase HPLC. ESI-MS m/z calc. 364.1. found 365.1 (M+1)⁺; retention time 2.70 minutes.

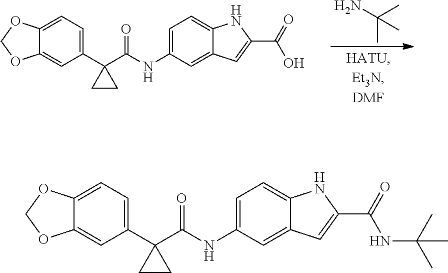

### 5-(1-(benzo[d][1,3]dioxol-5-yl)cyclopropanecarboxamido)-N-tert-butyl-1H-indole-2-carboxamide

5-(1-(Benzo[d][1,3]dioxol-5-yl)cyclopropane-carboxamido)-1H-indole-2-carboxylic acid (36 mg, 0.10 mmol) and 2-methylpropan-2-amine (8.8 mg, 0.12 mmol) were dissolved in N,N-dimethylformamide (1.0 mL) containing triethylamine (28 μL, 0.20 mmol). 0-(7-Azabenzotriazol-1-yl)-N,N,N′,N′-tetramethyluronium hexafluorophosphate (46 mg, 0.12 mmol) was added to the mixture and the resulting solution was allowed to stir for 3 hours. The mixture was filtered and purified by reverse phase HPLC to yield 5-(1-(benzo[d][1,3]dioxol-5-yl)cyclopropanecarboxamido)-N-tert-butyl-1H-indole-2-carboxamide. ESI-MS m/z calc. 419.2. found 420.3 (M+1)⁺; retention time 3.12 minutes.

### Example 81

### N-(3-Amino-2-tert-butyl-1H-indol-5-yl)-1-(benzo[d][1,3]dioxol-5-yl)cyclopropanecarboxamide

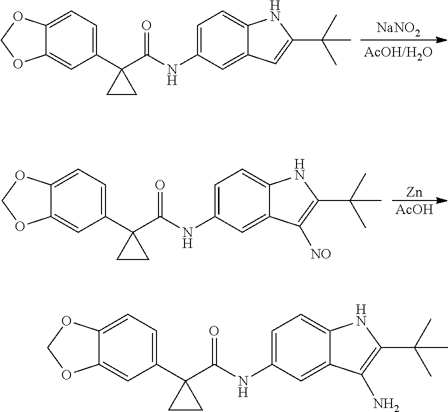

A solution of 1-(benzo[d][1,3]dioxol-5-yl)-N-(2-tert-butyl-1H-indol-5-yl)cyclopropane carboxamide (50 mg, 0.13 mmol) was dissolved in AcOH (2 mL) and warmed to 45° C. To the mixture was added a solution of NaNO₂ (9 mg) in H₂O (0.03 mL). The mixture was allowed to stir for 30 min at 45° C. before the precipitate was collected and washed with Et₂O. This material was used in the next step without further purification. To the crude material, 1-(benzo[d][1,3]dioxol-5-yl)-N-(2-tert-butyl-3-nitroso-1H-indol-5-yl)cyclopropanecarboxamide, was added AcOH (2 mL) and Zn dust (5 mg). The mixture was allowed to stir for 1 h at ambient temperature. EtOAc and H₂O were added to the mixture. The layers were separated and the organic layer was washed with sat. aq. NaHCO₃, dried over MgSO₄, and concentrated in vacuo. The residue was taken up in DMF (1 mL) and was purified using prep-HPLC. LCMS: m/z 392.3; retention time of 2.18 min.

### Example 82

### 1-(Benzo[d][1,3]dioxol-5-yl)-N-(2-tert-butyl-3-(methylsulfonyl)-1H-indol-5-yl)cyclopropanecarboxamide

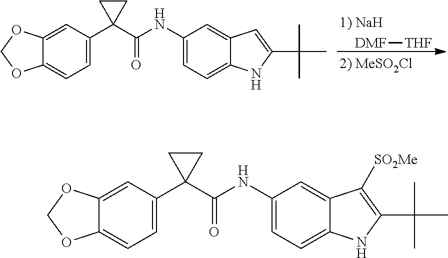

### 1-(Benzo[d][1,3]dioxol-5-yl)-N-(2-tert-butyl-3-(methylsulfonyl)-1H-indol-5-yl)cyclopropanecarboxamide

To a solution of 1-(benzo[d][1,3]dioxol-5-yl)-N-(2-tert-butyl-1H-indol-5-yl)cyclopropanecarboxamide (120 mg, 0.31 mmol) in anhydrous DMF-THF (3.3 mL, 1:9) was added NaH (60% in mineral oil, 49 mg, 1.2 mmol) at room temperature. After 30 min under N₂, the suspension was cooled down to −15° C. and a solution of methanesulfonyl chloride (1.1 eq.) in DMF (0.5 mL) was added dropwise. The reaction mixture was stirred for 30 min at −15° C. then for 6 h at room temperature. Water (0.5 mL) was added at 0° C., solvent was removed, and the residue was diluted with MeOH, filtrated and purified by preparative HPLC to give 1-(Benzo[d][1,3]dioxol-5-yl)-N-(2-tert-butyl-3-(methylsulfonyl)-1H-indol-5-yl)cyclopropanecarboxamide. ¹H NMR (400 MHz, DMSO) δ 11.6 (s, 1H), 8.7 (s, 1H), 7.94 (d, J=1.7 Hz, 1H), 7.38 (d, J=8.7 Hz, 1H), 7.33 (dd, J1=1.9 Hz, J2=8.7 Hz, 1H), 7.03 (d, J=1.7 Hz, 1H), 6.95 (dd, J1=1.7 Hz, J2=8.0 Hz, 1H), 6.90 (d, J=8.0 Hz, 1H), 6.02 (s, 2H), 3.07 (s, 3H), 1.56-1.40 (m, 9H), 1.41 (dd, J1=4.0 Hz, J2=6.7 Hz, 2H), 1.03 (dd, J1=4.0 Hz, J2=6.7 Hz, 2H). MS (ESI) m/e (M+H⁺) 455.5.

### Example 83

### 1-(Benzo[d][1,3]dioxol-5-yl)-N-(3-phenyl-1H-indol-5-yl)cyclopropane carboxamide

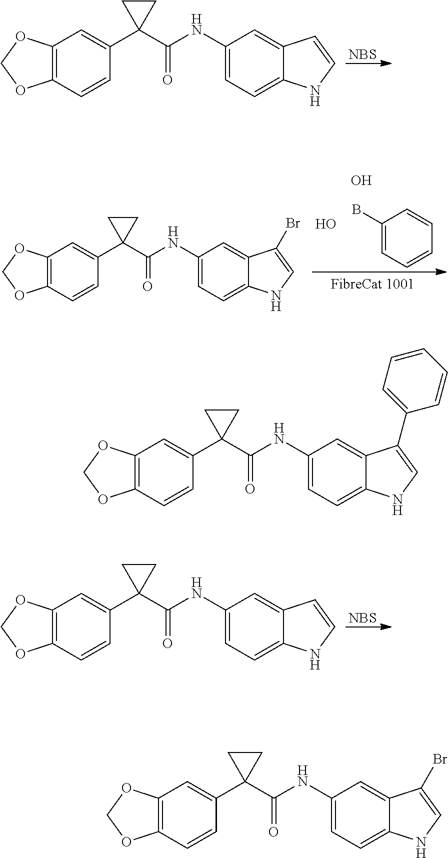

### 1-(Benzo[d][1,3]dioxol-5-yl)-N-(3-bromo-1H-indol-5-yl)cyclopropanecarboxamide

Freshly recrystallized N-bromosuccinimde (0.278 g, 1.56 mmol) was added portionwise to a solution of 1-(benzo[d][1,3]dioxol-5-yl)-N-(1H-indol-5-yl)cyclopropanecarboxamide (0.500 g, 1.56 mmol) in N,N-dimethylformamide (2 mL) over 2 minutes. The reaction mixture was protected from light and was stirred bar for 5 minutes. The resulting green solution was poured into 40 mL of water. The grey precipitate which formed was filtered and washed with water to yield 1-(benzo[d][1,3]dioxol-5-yl)-N-(3-bromo-1H-indol-5-yl)cyclopropanecarboxamide (0.564 g, 91%). ESI-MS m/z calc. 398.0. found 399.3 (M+1)⁺. Retention time of 3.38 minutes. ¹H NMR (400 MHz, DMSO-d6) 11.37 (s, 1H), 8.71 (s, 1H), 7.67 (d, J=1.8 Hz, 1H), 7.50 (d, J=2.6 Hz, 1H), 7.29 (d, J=8.8 Hz, 1H), 7.22 (dd, J=2.0, 8.8 Hz, 1H), 7.02 (d, J=1.6 Hz, 1H), 6.96-6.88 (m, 2H), 6.03 (s, 2H), 1.43-1.40 (m, 2H), 1.09-1.04 (m, 2H).

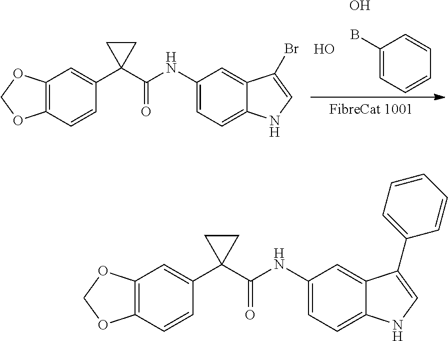

### 1-(Benzo[d][1,3]dioxol-5-yl)-N-(3-phenyl-1H-indol-5-yl)cyclopropanecarboxamide

Phenyl boronic acid (24.6 mg, 0.204 mmol) was added to a solution of 1-(benzo[d][1,3]-dioxol-5-yl)-N-(3-bromo-1H-indol-5-yl)cyclopropanecarboxamide (39.9 mg, 0.100 mmol) in ethanol (1 mL) containing FibreCat 1001 (6 mg) and 1M aqueous potassium carbonate (0.260 mL). The reaction mixture was then heated at 130° C. in a microwave reactor for 20 minutes. The crude product was then purified by preparative HPLC utilizing a gradient of 0-99% acetonitrile in water containing 0.05% trifluoroacetic acid to yield 1-(benzo[d][1,3]dioxol-5-yl)-N-(3-phenyl-1H-indol-5-yl)cyclopropane carboxamide. ESI-MS m/z calc. 396.2. found 397.3 (M+1)⁺. Retention time of 3.52 minutes. ¹H NMR (400 MHz, DMSO-d6) 11.27 (d, J=1.9 Hz, 1H), 8.66 (s, 1H), 8.08 (d, J=1.6 Hz, 1H), 7.65-7.61 (m, 3H), 7.46-7.40 (m, 2H), 7.31 (d, J=8.7 Hz, 1H), 7.25-7.17 (m, 2H), 7.03 (d, J=1.6 Hz, 1H), 6.98-6.87 (m, 2H), 6.02 (s, 2H), 1.43-1.39 (m, 2H), 1.06-1.02 (m, 2H).

### Example 84

### 1-(Benzo[d][1,3]dioxol-5-yl)-N-(2-tert-butyl-3-cyano-1H-indol-5-yl)cyclopropanecarboxamide

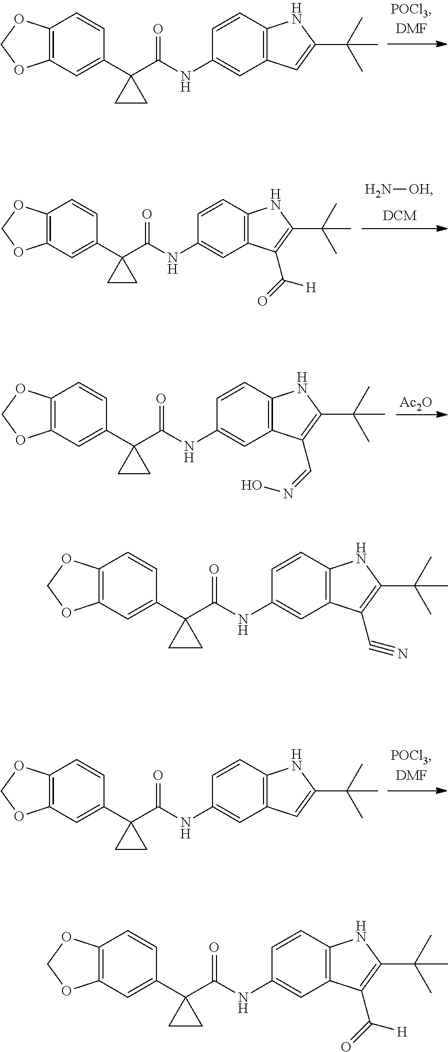

### 1-(Benzo[d][1,3]dioxol-5-yl)-N-(2-tert-butyl-3-formyl-1H-indol-5-yl)cyclopropane-carboxamide

POCl₃ (12 g, 80 mmol) was added dropwise to DMF (40 mL) held at −20° C. After the addition was complete, the reaction mixture was allowed to warm to 0° C. and was stirred for 1 h. 1-(Benzo[d][1,3]dioxol-5-yl)-N-(2-tert-butyl-1H-indol-5-yl)cyclopropanecarboxamide (3.0 g, 8.0 mmol) was added and the mixture was warmed to 25° C. After stirring for 30 minutes the reaction mixture was poured over ice and stirred for 2 h. The mixture was then heated at 100° C. for 30 min. The mixture was cooled and the solid precipitate was collected and washed with water. The solid was then dissolved in 200 mL dichloromethane and washed with 200 mL of a saturated aq. NaHCO₃. The organics were dried over Na₂SO₄ and evaporated to yield 1-(benzo[d][1,3]dioxol-5-yl)-N-(2-tert-butyl-3-formyl-1H-indol-5-yl)cyclopropane-carboxamide (2.0 g, 61%). ESI-MS m/z calc. 404.5. found 405.5 (M+1)⁺; retention time 3.30 minutes. ¹H NMR (400 MHz, DMSO-d6) δ 11.48 (s, 1H), 10.39 (s, 1H), 8.72 (s, 1H), 8.21 (s, 1H), 7.35-7.31 (m, 2H), 7.04-7.03 (m, 1H), 6.97-6.90 (m, 2H), 6.03 (s, 2H), 1.53 (s, 9H), 1.42-1.39 (m, 2H), 1.05-1.03 (m, 2H).

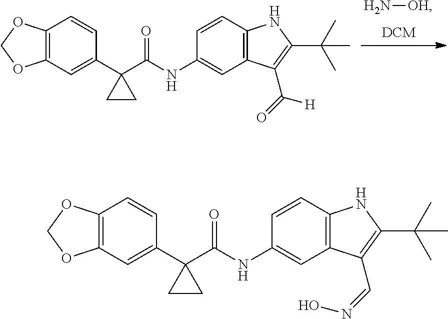

### (Z)-1-(Benzo[d][1,3]dioxol-5-yl)-N-(2-tert-butyl-3-((hydroxyimino)methyl)-1H-indol-5-yl)cyclopropanecarboxamide

To a solution of 1-(benzo[d][1,3]dioxol-5-yl)-N-(2-tert-butyl-3-formyl-1H-indol-5-yl)cyclopropanecarboxamide (100 mg, 0.25 mmol) in dichloromethane (5 mL) was added hydroxylamine hydrochloride (21 mg, 0.30 mmol). After stirring for 48 h, the mixture was evaporated to dryness and purified by column chromatography (0-100% ethyl acetate/hexanes) to yield (Z)-1-(benzo[d][1,3]dioxol-5-yl)-N-(2-tert-butyl-3-((hydroxyimino)methyl)-1H-indol-5-yl)cyclopropanecarboxamide (81 mg, 77%). ESI-MS m/z calc. 419.5. found 420.5 (M+1)⁺; retention time 3.42 minutes. ¹H NMR (400 MHz, DMSO-d6) δ 10.86 (s, 0.5H), 10.55 (s, 0.5H), 8.56-8.50 (m, 2H), 8.02 (m, 1H), 7.24-7.22 (m, 1H), 7.12-7.10 (m, 1H), 7.03 (m, 1H), 6.96-6.90 (m, 2H), 6.03 (s, 2H), 1.43 (s, 9H), 1.40-1.38 (m, 2H), 1.04-1.01 (m, 2H).

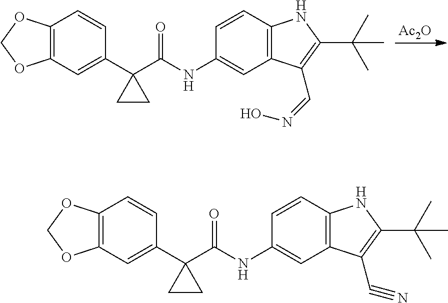 [SMILES: "Ac2O" ; CC(C)(C)c1[nH]c2ccc(NC(=O)C3(c4ccc5c(c4)OCO5)CC3)cc2c1/C=N\O ; CC(C)(C)c1[nH]c2ccc(NC(=O)C3(c4ccc5c(c4)OCO5)CC3)cc2c1C#N]

### 1-(Benzo[d][1,3]dioxol-5-yl)-N-(2-tert-butyl-3-cyano-1H-indol-5-yl)cyclopropane-carboxamide

(Z)-1-(Benzo[d][1,3]dioxol-5-yl)-N-(2-tert-butyl-3-((hydroxyimino)-methyl)-1H-indol-5-yl)cyclopropanecarboxamide (39 mg, 0.090 mmol) was dissolved in acetic anhydride (1 mL) and heated at reflux for 3 h. The mixture was cooled in an ice bath and the precipitate was collected and washed with water. The solid was further dried under high vacuum to yield 1-(benzo[d][1,3]dioxol-5-yl)-N-(2-tert-butyl-3-cyano-1H-indol-5-yl)cyclopropanecarboxamide. ESI-MS m/z calc. 401.5. found 402.5 (M+1)⁺; retention time 3.70 minutes. ¹H NMR (400 MHz, DMSO-d6) δ 11.72 (s, 1H), 8.79 (s, 1H), 7.79 (s, 1H), 7.32 (m, 2H), 7.03-7.02 (m, 1H), 6.95-6.89 (m, 2H), 6.03 (s, 2H), 1.47 (s, 9H), 1.43-1.41 (m, 2H), 1.06-1.04 (m, 2H).

### Example 85

### 1-(Benzo[d][1,3]dioxol-5-yl)-N-(2-tert-butyl-3-methyl-1H-indol-5-yl)cyclopropanecarboxamide

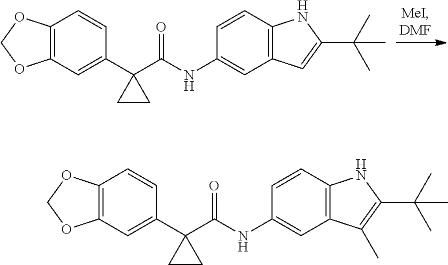 [SMILES: CC(C)(C)c1cc2cc(NC(=O)C3(c4ccc5c(c4)OCO5)CC3)ccc2[nH]1 ; Cc1c(C(C)(C)C)[nH]c2ccc(NC(=O)C3(c4ccc5c(c4)OCO5)CC3)cc12]

A solution of 1-(benzo[d][1,3]dioxol-5-yl)-N-(2-tert-butyl-1H-indol-5-yl)cyclopropanecarboxamide (75 mg, 0.20 mmol) and iodomethane (125 μL, 2.0 mmol) in N,N-dimethylformamide (1 mL) was heated at 120° C. in a sealed tube for 24 h. The reaction was filtered and purified by reverse phase HPLC. ESI-MS m/z calc. 390.5. found 391.3 (M+1)⁺; retention time 2.04 minutes. ¹H NMR (400 MHz, DMSO-d6) δ 10.30 (s, 1H), 8.39 (s, 1H), 7.51 (m, 1H), 7.13-7.11 (m, 1H), 7.03-6.90 (m, 4H), 6.03 (s, 2H), 2.25 (s, 3H), 1.40-1.38 (m, 11H), 1.03-1.01 (m, 2H).

### Example 86

### 1-(Benzo[d][1,3]dioxol-5-yl)-N-(2-tert-butyl-3-(2-hydroxyethyl)-1H-indol-5-yl)cyclopropanecarboxamide

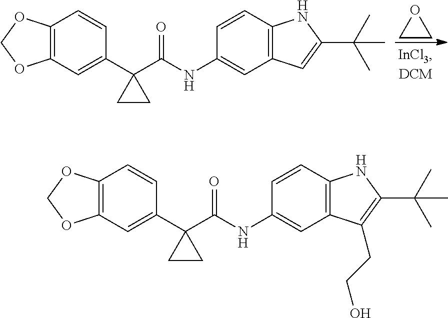

Approximately 100 μL of ethylene dioxide was condensed in a reaction tube at −78° C. A solution of 1-(benzo[d][1,3]dioxol-5-yl)-N-(2-tert-butyl-1H-indol-5-yl)cyclopropanecarboxamide (200 mg, 0.50 mmol) and indium trichloride (20 mg, 0.10 mmol) in dichloromethane (2 mL) was added and the reaction mixture was irradiated in the microwave for 20 min at 100° C. The volatiles were removed and the residue was purified by column chromatography (0-100% ethyl acetate/hexanes) to give 1-(benzo[d][1,3]dioxol-5-yl)-N-(2-tert-butyl-3-(2-hydroxyethyl)-1H-indol-5-yl)cyclopropanecarboxamide (5 mg, 3%). ESI-MS m/z calc. 420.5. found 421.3 (M+1)⁺; retention time 1.67 minutes. ¹H NMR (400 MHz, CD₃CN) 8.78 (s, 1H), 7.40 (m, 1H), 7.33 (s, 1H), 7.08 (m, 1H), 6.95-6.87 (m, 3H), 6.79 (m, 1H), 5.91 (s, 2H), 3.51 (dd, J=5.9, 7.8 Hz, 2H), 2.92-2.88 (m, 2H), 2.64 (t, J=5.8 Hz, 1H), 1.50 (m, 2H), 1.41 (s, 9H), 1.06 (m, 2H).

### Example 87

### 2-(5-(1-(Benzo[d][1,3]dioxol-5-yl)cyclopropanecarboxamido)-1H-indol-2-yl)acetic acid

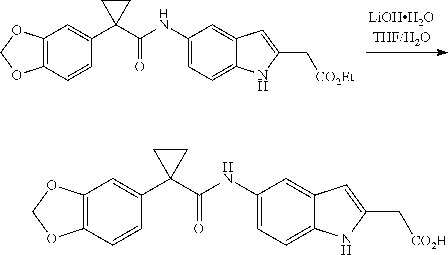 [SMILES: CCOC(=O)Cc1cc2cc(NC(=O)C3(c4ccc5c(c4)OCO5)CC3)ccc2[nH]1 ; O=C(O)Cc1cc2cc(NC(=O)C3(c4ccc5c(c4)OCO5)CC3)ccc2[nH]1]

To a solution of ethyl 2-(5-(1-(benzo[d][1,3]dioxol-5-yl)cyclopropanecarboxamido)-1H-indol-2-yl)acetate (0.010 g, 0.025 mmol) in THF (0.3 mL) were added LiOH.H₂O (0.002 g, 0.05 mmol) and water (0.15 mL) were added. The mixture was stirred at room temperature for 2 h. dichloromethane (3 mL) was added to the reaction mixture and the organic layer was washed with 1N HCl (2×1.5 mL) and water (2×1.5 mL). The organic layer was dried over Na₂SO₄ and filtered. The filtrate was evaporated under reduced pressure to give 2-(5-(1-(benzo[d][1,3]dioxol-5-yl)cyclopropanecarboxamido)-1H-indol-2-yl)-acetic acid. ¹H NMR (400 MHz, DMSO-d6) δ 12.53 (s, 1H), 10.90 (s, 1H), 8.42 (s, 1H), 7.57 (s, 1H), 7.17 (d, J=8.6 Hz, 1H), 7.05-6.90 (m, 4H), 6.17 (s, 1H), 6.02 (s, 2H), 3.69 (s, 2H), 1.41-1.39 (m, 2H), 1.04-1.02 (m, 2H).

### Example 88

### 5-(1-(Benzo[d][1,3]dioxol-5-yl)cyclopropanecarboxamido)-2-tert-butyl-1H-indole-7-carboxylic acid

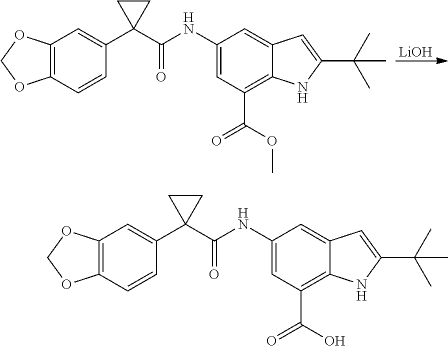

Methyl 5-(1-(benzo[d][1,3]dioxol-5-yl)cyclopropanecarboxamido)-2-tert-butyl-1H-indole-7-carboxylate (30 mg, 0.069 mmol) was dissolved in a mixture of 1,4-dioxane (1.5 mL) and water (2 mL) containing a magnetic star bar and lithium hydroxide (30 mg, 0.71 mmol). The resulting solution was stirred at 70° C. for 45 minutes. The crude product was then acidified with 2.6 M hydrochloric acid and extracted three times with an equivalent volume of dichloromethane. The dichloromethane extracts were combined, dried over sodium sulfate, filtered, and evaporated to dryness. The residue was dissolved in a minimum of N,N-dimethylformamide and then purified by preparative HPLC using a gradient of 0-99% acetonitrile in water containing 0.05% trifluoroacetic acid to yield 5-(1-(benzo[d][1,3]dioxol-5-yl)cyclopropanecarboxamido)-2-tert-butyl-1H-indole-7-carboxylic acid. ESI-MS m/z calc. 434.2. found 435.5. Retention time of 1.85 minutes. ¹H NMR (400 MHz, DMSO-d6) δ 13.05 (s, 1H), 9.96 (d, J=1.6 Hz, 1H), 7.89 (d, J=1.9 Hz, 1H), 7.74 (d, J=2.0 Hz, 1H), 7.02 (d, J=1.6 Hz, 1H), 6.96-6.88 (m, 2H), 6.22 (d, J=2.3 Hz, 1H), 6.02 (s, 2H), 1.43-1.40 (m, 2H), 1.37 (s, 9H), 1.06-1.02 (m, 2H).

### Example 89

### 1-(Benzo[d][1,3]dioxol-5-yl)-N-(2-tert-butyl-1-(1,3-dihydroxypropan-2-yl)-1H-indol-5-yl)cyclopropanecarboxamide

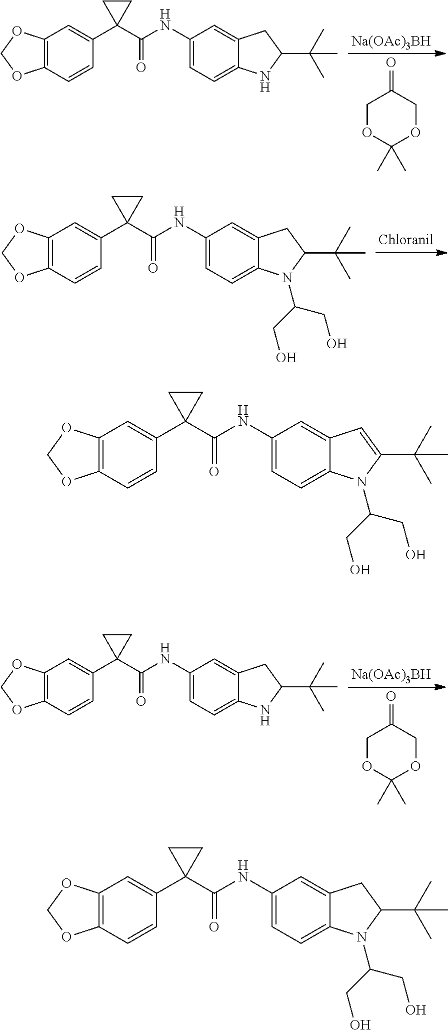

### 1-(Benzo[d][1,3]dioxol-5-yl)-N-(2-tert-butyl-1-(1,3-dihydroxypropan-2-yl)indolin-5-yl)cyclopropanecarboxamide

1-(Benzo[d][1,3]dioxol-5-yl)-N-(2-tert-butylindolin-5-yl)cyclopropanecarboxamide (50 mg, 0.13 mmol) was dissolved in dichloroethane (0.20 mL) and 2,2-dimethyl-1,3-dioxan-5-one (0.20 mL). Trifluoroacetic acid was added (0.039 mL) and the resulting solution was allowed to stir for 20 minutes. Sodium triacetoxyborohydride was added (55 mg, 0.26 mmol) and the reaction mixture was stirred for 30 minutes. The crude reaction mixture was then evaporated to dryness, dissolved in N,N-dimethylformamide and purified by preparative HPLC using a gradient of 0-99% acetonitrile in water containing 0.05% trifluoroacetic acid.

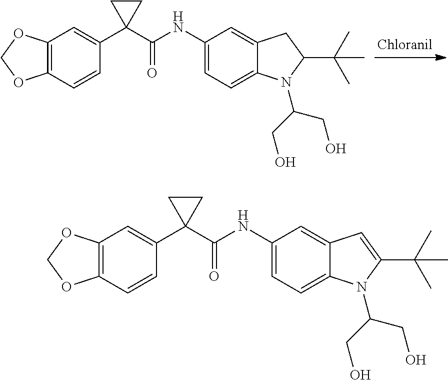 [SMILES: CC(C)(C)C1Cc2cc(NC(=O)C3(c4ccc5c(c4)OCO5)CC3)ccc2N1C(CO)CO ; CC(C)(C)c1cc2cc(NC(=O)C3(c4ccc5c(c4)OCO5)CC3)ccc2n1C(CO)CO]

### 1-(Benzo[d][1,3]dioxol-5-yl)-N-(2-tert-butyl-1-(1,3-dihydroxypropan-2-yl)-1H-indol-5-yl)cyclopropanecarboxamide

1-(Benzo[d][1,3]dioxol-5-yl)-N-(2-tert-butyl-1-(1,3-dihydroxypropan-2-yl)indolin-5-yl)cyclopropanecarboxamide (40.3 mg, 0.0711 mmol as the trifluoracetic acid salt) was dissolved in toluene (1 mL). To the resulting solution was added 2,3,5,6-tetrachlorocyclohexa-2,5-diene-1,4-dione (35 mg, 0.14 mmol). The resulting suspension was heated at 100° C. in an oil bath for 10 minutes. The crude product was then evaporated to dryness, dissolved in a 1 mL of N,N-dimethylformamide and purified by purified by preparative HPLC using a gradient of 0-99% acetonitrile in water containing 0.05% trifluoroacetic acid to yield 1-(benzo[d][1,3]dioxol-5-yl)-N-(2-tert-butyl-1-(1,3-dihydroxypropan-2-yl)-1H-indol-5-yl)cyclopropanecarboxamide. ESI-MS m/z calc. 450.2. found 451.5 (M+1)⁺. Retention time of 1.59 minutes.

### Example 90

### N-(7-(Aminomethyl)-2-tert-butyl-1H-indol-5-yl)-1-(benzo[d][1,3]-dioxol-5-yl)cyclopropanecarboxamide

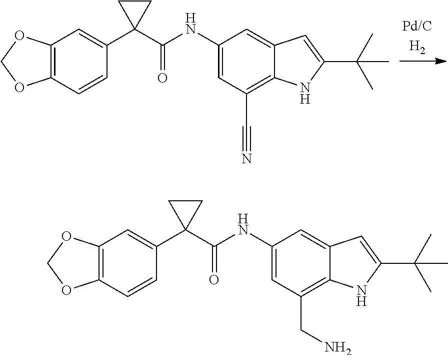 [SMILES: CC(C)(C)c1cc2cc(NC(=O)C3(c4ccc5c(c4)OCO5)CC3)cc(C#N)c2[nH]1 ; CC(C)(C)c1cc2cc(NC(=O)C3(c4ccc5c(c4)OCO5)CC3)cc(CN)c2[nH]1]

### N-(7-(Aminomethyl)-2-tert-butyl-1H-indol-5-yl)-1-(benzo[d][1,3]dioxol-5-yl)cyclopropanecarboxamide

1-(Benzo[d][1,3]dioxol-5-yl)-N-(2-tert-butyl-7-cyano-1H-indol-5-yl)cyclopropanecarboxamide (375 mg, 0.934 mmol) was dissolved in 35 mL of ethyl acetate. The solution was recirculated through a continuous flow hydrogenation reactor containing 10% palladium on carbon at 100° C. under 100 bar of hydrogen for 8 h. The crude product was then evaporated to dryness and purified on 12 g of silica gel utilizing a gradient of 0-100% ethyl acetate (containing 0.5% triethylamine) in hexanes to yield N-(7-(aminomethyl)-2-tert-butyl-1H-indol-5-yl)-1-(benzo[d][1,3]-dioxol-5-yl)-cyclopropanecarboxamide (121 mg, 32%). ESI-MS m/z calc. 405.2. found 406.5 (M+1)⁺. Retention time of 1.48 minutes.

### Example 91

### 5-(1-(Benzo[d][1,3]dioxol-5-yl)cyclopropanecarboxamido)-2-tert-butyl-1H-indole-7-carboxamide

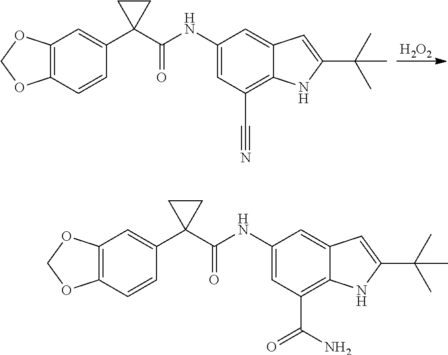

### 5-(1-(Benzo[d][1,3]dioxol-5-yl)cyclopropanecarboxamido)-2-tert-butyl-1H-indole-7-carboxamide

1-(Benzo[d][1,3]dioxol-5-yl)-N-(2-tert-butyl-7-cyano-1H-indol-5-yl)-cyclopropanecarboxamide (45 mg, 0.11 mmol) was suspended in a mixture of methanol (1.8 mL), 30% aqueous hydrogen peroxide (0.14 mL, 4.4 mmol) and 10% aqueous sodium hydroxide (0.150 mL). The resulting suspension was stirred for 72 h at room temperature. The hydrogen peroxide was then quenched with sodium sulfite. The reaction mixture was diluted with 0.5 mL of N,N-dimethylformamide, filtered, and purified by preparative HPLC using a gradient of 0-99% acetonitrile in water containing 0.05% trifluoroacetic acid to yield 5-(1-(benzo[d][1,3]dioxol-5-yl)cyclopropane-carboxamido)-2-tert-butyl-1H-indole-7-carboxamide. ESI-MS m/z calc. 419.2. found 420.3 (M+1)⁺. Retention time of 1.74 minutes.

### Example 92

### 1-(Benzo[d][1,3]dioxol-5-yl)-N-(2-tert-butyl-7-(methylsulfonamido-methyl)-1H-indol-5-yl)cyclopropanecarboxamide

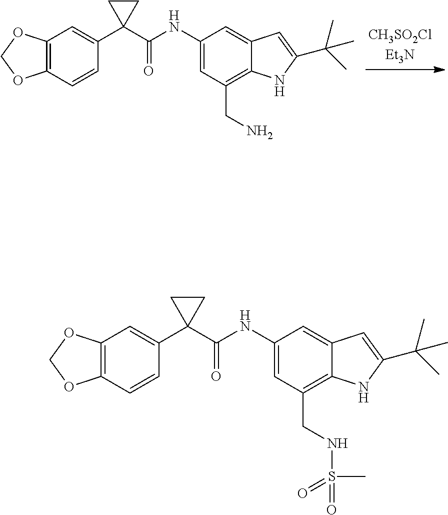 [SMILES: CC(C)(C)c1cc2cc(NC(=O)C3(c4ccc5c(c4)OCO5)CC3)cc(CN)c2[nH]1 ; CC(C)(C)c1cc2cc(NC(=O)C3(c4ccc5c(c4)OCO5)CC3)cc(CNS(C)(=O)=O)c2[nH]1]

### 1-(Benzo[d][1,3]dioxol-5-yl)-N-(2-tert-butyl-7-(methylsulfonamidomethyl)-1H-indol-5-yl)cyclopropanecarboxamide

N-(7-(Aminomethyl)-2-tert-butyl-1H-indol-5-yl)-1-(benzo[d][1,3]dioxol-5-yl)cyclopropanecarboxamide (20 mg, 0.049 mmol) was dissolved in DMF (0.5 mL) containing triethylamine (20.6 μL, 0.147 mmol) and a magnetic stir bar. Methanesulfonyl chloride (4.2 μL, 0.054 mmol) was then added to the reaction mixture. The reaction mixture was allowed to stir for 12 h at room temperature. The crude product was purified by preparative HPLC using a gradient of 0-99% acetonitrile in water containing 0.05% trifluoroacetic acid to yield 1-(benzo[d][1,3]dioxol-5-yl)-N-(2-tert-butyl-7-(methylsulfonamidomethyl)-1H-indol-5-yl)cyclopropanecarboxamide. ESI-MS m/z calc. 483.2. found 484.3 (M+1)⁺. Retention time of 1.84 minutes.

### Example 93

### N-(7-(Acetamidomethyl)-2-tert-butyl-1H-indol-5-yl)-1-(benzo[d][1,3]-dioxol-5-yl)cyclopropanecarboxamide

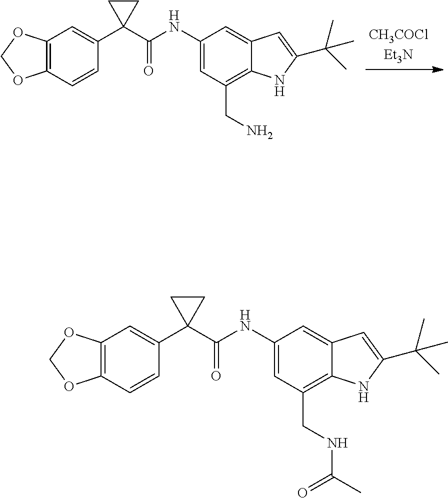 [SMILES: CC(C)(C)c1cc2cc(NC(=O)C3(c4ccc5c(c4)OCO5)CC3)cc(CN)c2[nH]1 ; CC(=O)NCc1cc(NC(=O)C2(c3ccc4c(c3)OCO4)CC2)cc2cc(C(C)(C)C)[nH]c12]

N-(7-(Aminomethyl)-2-tert-butyl-1H-indol-5-yl)-1-(benzo[d][1,3]dioxol-5-yl)cyclopropanecarboxamide (20 mg, 0.049 mmol) was dissolved in DMF (0.5 mL) containing triethylamine (20.6 μL, 0.147 mmol) and a magnetic stir bar. Acetyl chloride (4.2 μL, 0.054 mmol) was then added to the reaction mixture. The reaction mixture was allowed to stir for 16 h at room temperature. The crude product was purified by preparative HPLC using a gradient of 0-99% acetonitrile in water containing 0.05% trifluoroacetic acid to yield N-(7-(acetamidomethyl)-2-tert-butyl-1H-indol-5-yl)-1-(benzo[d][1,3]dioxol-5-yl)cyclopropanecarboxamide. ESI-MS m/z calc. 447.2. found 448.3 (M+1)⁺. Retention time of 1.76 minutes.

### Example 94

### N-(1-Acetyl-2-tert-butyl-1H-indol-5-yl)-1-(benzo[d][1,3]dioxol-5-yl)-cyclopropanecarboxamide

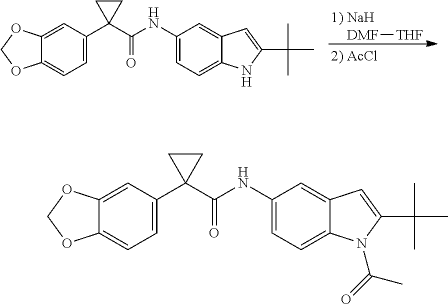

To a solution of 1-(benzo[d][1,3]dioxol-5-yl)-N-(2-tert-butyl-1H-indol-5-yl)cyclopropanecarboxamide (120 mg, 0.31 mmol) in anhydrous DMF-THF (3.3 mL, 1:9) was added NaH (60% in mineral oil, 49 mg, 1.2 mmol) at room temperature. After 30 min under N₂, the suspension was cooled down to −15° C. and a solution of acetyl chloride (1.1 eq.) in DMF (0.5 mL) was added dropwise. The reaction mixture was stirred for 30 min at −15° C. then for 6 h at room temperature. Water (0.5 mL) was added at 0° C., solvent was removed, and the residue was diluted with MeOH, filtrated and purified by preparative HPLC to give N-(1-acetyl-2-tert-butyl-1H-indol-5-yl)-1-(benzo[d][1,3]dioxol-5-yl)cyclo-propanecarboxamide. ¹H NMR (400 MHz, DMSO) δ 8.9 (s, 1H), 7.74 (d, J=2.1 Hz, 1H), 7.54 (d, J=9.0 Hz, 1H), 7.28 (dd, J1=2.1 Hz, J2=9.0 Hz, 1H), 7.01 (d, J=1.5 Hz, 1H), 6.93 (dd, J1=1.7 Hz, J2=8.0 Hz, 1H), 6.89 (d, J=8.0 Hz, 1H), 6.54 (bs, 1H), 6.02 (s, 2H), 2.80 (s, 3H), 1.42-1.40 (m, 11H), 1.06-1.05 (m, 2H). MS (ESI) m/z (M+H⁺) 419.3.

### Example 95

### N-(1-(2-Acetamidoethyl)-2-tert-butyl-6-fluoro-1H-indol-5-yl)-1-(2,2-difluorobenzo[d][1,3]dioxol-5-yl)cyclopropanecarboxamide

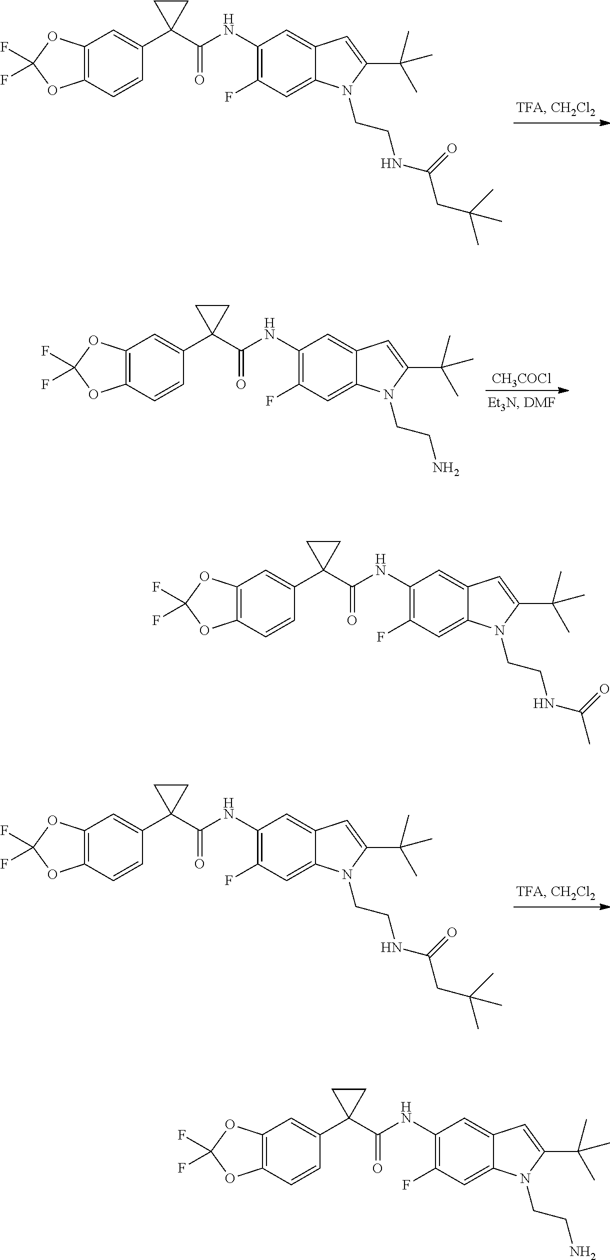

### N-(1-(2-Aminoethyl)-2-tert-butyl-6-fluoro-1H-indol-5-yl)-1-(2,2-difluorobenzo-[d][1,3]dioxol-5-yl)cyclopropanecarboxamide

To a solution of tert-butyl 2-(2-tert-butyl-5-(1-(2,2-difluorobenzo[d][1,3]dioxol-5-yl)cyclopropanecarboxamido)-6-fluoro-1H-indol-1-yl)ethylcarbamate (620 mg, 1.08 mmol) in CH₂Cl₂ (8 mL) was added TFA (2 mL). The reaction was stirred at room temperature for 1.5 h before being neutralized with solid NaHCO₃. The solution was partitioned between H₂O and CH₂Cl₂. The organic layer was dried over MgSO₄, filtered and concentrated to yield the product as a cream colored solid (365 mg, 71%). ¹H NMR (400 MHz, DMSO-d6) δ 8.38 (s, 1H), 7.87 (br s, 3H, NH₃⁺), 7.52 (s, 1H), 7.45-7.38 (m, 3H), 7.32 (dd, J=8.3, 1.5 Hz, 1H), 6.21 (s, 1H), 4.46 (m, 2H), 3.02 (m, 2H), 1.46 (m, 2H), 1.41 (s, 9H), 1.14 (m, 2H). HPLC ret. time 1.66 min, 10-99% CH₃CN, 3 min run; ESI-MS 474.4 m/z (M+H⁺).

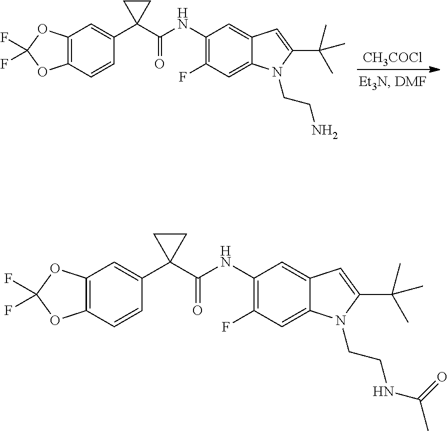

### N-(1-(2-Acetamidoethyl)-2-tert-butyl-6-fluoro-1H-indol-5-yl)-1-(2,2-difluorobenzo[d][1,3]dioxol-5-yl)cyclopropanecarboxamide

To a solution of N-(1-(2-aminoethyl)-2-tert-butyl-6-fluoro-1H-indol-5-yl)-1-(2,2-difluorobenzo[d][1,3]dioxol-5-yl)cyclopropane-carboxamide (47 mg, 0.10 mmol) and Et₃N (28 μL, 0.20 mmol) in DMF (1 mL) was added acetyl chloride (7.1 μL, 0.10 mmol). The mixture was stirred at room temperature for 1 h before being filtered and purified by reverse phase HPLC (10-99% CH₃CN/H₂O) to yield N-(1-(2-acetamidoethyl)-2-tert-butyl-6-fluoro-1H-indol-5-yl)-1-(2,2-difluorobenzo[d][1,3]dioxol-5-yl)cyclopropanecarboxamide. ¹H NMR (400 MHz, DMSO-d6) δ 8.35 (s, 1H), 8.15 (t, J=5.9 Hz, 1H), 7.53 (s, 1H), 7.43-7.31 (m, 4H), 6.17 (s, 1H), 4.22 (m, 2H), 3.30 (m, 2H), 1.85 (s, 3H), 1.47 (m, 2H), 1.41 (s, 9H), 1.13 (m, 2H). HPLC ret. time 2.06 min, 10-99% CH₃CN, 3 min run; ESI-MS 516.4 m/z (M+H⁺).

### Example 96

### 1-(Benzo[d][1,3]dioxol-5-yl)-N-(2-tert-butyl-1-(2-hydroxy-3-methoxy-propyl)-1H-indol-5-yl)cyclopropenecarboxamide

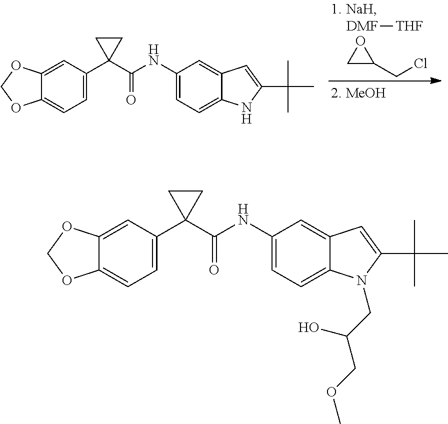

1-(Benzo[d][1,3]dioxol-5-yl)-N-(2-tert-butyl-1H-indol-5-yl)cyclopropanecarboxamide (320 mg, 0.84 mmol) was dissolved in a mixture composed of anhydrous DMF (0.5 mL) and anhydrous THF (5 mL) under N₂. NaH (60% in mineral oil, 120 mg, 3.0 mmol) was added at room temperature. After 30 min of stirring, the reaction mixture was cooled to −15° C. before a solution of epichlorohydrin (79 μL, 1.0 mmol) in anhydrous DMF (1 mL) was added dropwise. The reaction mixture was stirred for 15 min at −15° C., then for 8 h at room temperature. MeOH (1 mL) was added and the mixture was heated for 10 min at 105° C. in the microwave oven. The mixture was cooled, filtered and purified by preparative HPLC to give 1-(benzo[d][1,3]dioxol-5-yl)-N-(2-tert-butyl-1-(2-hydroxy-3-methoxy-propyl)-1H-indol-5-yl)cyclopropanecarboxamide. ¹H NMR (400 MHz, DMSO-d6) 8.44 (s, 1H), 7.59 (d, J=1.9 Hz, 1H), 7.31 (d, J=8.9 Hz, 1H), 7.03 (dd, J=8.7, 1.9 Hz, 2H), 6.95 (dd, J=8.0, 1.7 Hz, 1H), 6.90 (d, J=8.0 Hz, 1H), 6.16 (s, 1H), 6.03 (s, 2H), 4.33 (dd, J=15.0, 4.0 Hz, 1H), 4.19 (dd, J=15.0, 8.1 Hz, 1H), 4.02 (ddd, J=8.7, 4.8 Hz, 1H), 3.41-332 (m, 2H), 3.30 (s, 3H), 1.41 (s, 9H), 1.41-1.38 (m, 2H), 1.03 (dd, J=6.7, 4.0 Hz, 2H). MS (ESI) m/e (M+H⁺) 465.0.

### Example 97

### 1-(Benzo[d][1,3]dioxol-5-yl)-N-(2-tert-butyl-1-(2-hydroxy-3-(methyl-amino)propyl)-1H-indol-5-yl)cyclopropanecarboxamide

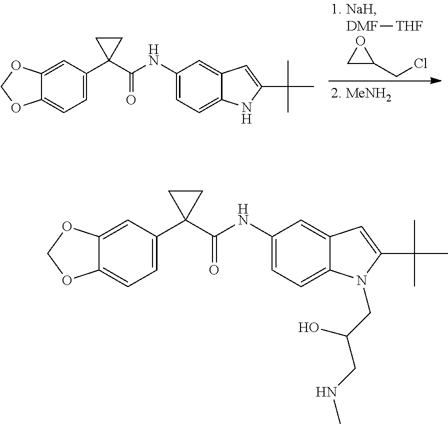

1-(Benzo[d][1,3]dioxol-5-yl)-N-(2-tert-butyl-1H-indol-5-yl)cyclopropanecarboxamide (320 mg, 0.84 mmol) was dissolved in a mixture composed of anhydrous DMF (0.5 mL) and anhydrous THF (5 mL) under N₂. NaH (60% in mineral oil, 120 mg, 3.0 mmol) was added at room temperature. After 30 min of stirring, the reaction mixture was cooled to −15° C. before a solution of epichlorohydrin (79 μL, 1.0 mmol) in anhydrous DMF (1 mL) was added dropwise. The reaction mixture was stirred for 15 min at −15° C., then for 8 h at room temperature. MeNH₂ (2.0 M in MeOH, 1.0 mL) was added and the mixture was heated for 10 min at 105° C. in the microwave oven. The mixture was cooled, filtered and purified by preparative HPLC to give 1-(benzo[d][1,3]dioxol-5-yl)-N-(2-tert-butyl-1-(2-hydroxy-3-(methylamino)propyl)-1H-indol-5-yl)cyclopropanecarboxamide. ¹H NMR (400 MHz, DMSO-d6) δ 8.50 (s, 1H), 7.60-7.59 (m, 1H), 7.35 (dd, J=14.3, 8.9 Hz, 1H), 7.10 (d, J=8.8 Hz, 1H), 1H), 6.94 (dd, J=8.0, 1.6 Hz, 1H), 6.91 (d, J=7.9 Hz, 1H), 6.20 (d, J=2.3 Hz, 1H), 6.03 (s, 2H), 2.82 (d, J=4.7 Hz, 1H), 2.72 (d, J=4.7 Hz, 1H), 2.55 (dd, J=5.2, 5.2 Hz, 1H), 2.50 (s, 3H), 1.43 (s, 9H), 1.39 (dd, J=6.4, 3.7 Hz, 2H), 1.04 (dd, J=6.5, 3.9 Hz, 2H). MS (ESI) m/e (M+H⁺) 464.0.

### Example 98

### (S)—N-(1-(3-Amino-2-hydroxypropyl)-2-tert-butyl-1H-indol-5-yl)-1-(2,2-difluorobenzo[d][1,3]dioxol-5-yl)cyclopropanecarboxamide

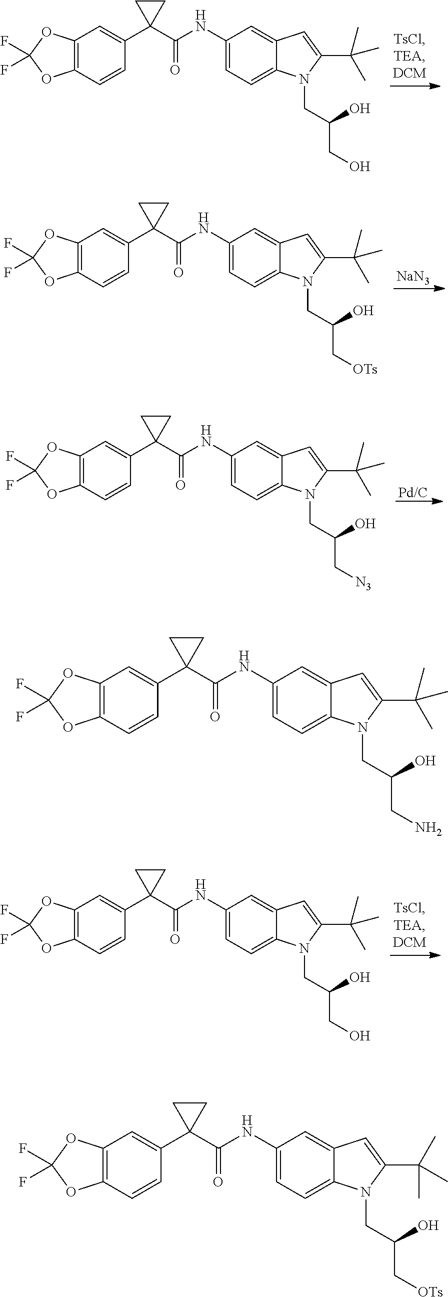

### (R)-3-(2-tert-Butyl-5-(1-(2,2-difluorobenzo[d][1,3]dioxol-5-yl)cyclopropanecarbox-amido)-1H-indol-1-yl)-2-hydroxypropyl-4-methylbenzenesulfonate

To a stirred solution of (R)—N-(2-tert-butyl-1-(2,3-dihydroxypropyl)-1H-indol-5-yl)-1-(2,2-difluoro-benzo[d][1,3]dioxol-5-yl)cyclopropanecarboxamide (3.0 g, 6.1 mmol) in dichloromethane (20 mL) was added triethylamine (2 mL) and para-toluenesulfonylchloride (1.3 g, 7.0 mmol). After 18 hours, the reaction mixture was partitioned between 10 mL of water and 10 mL of ethyl acetate. The organic layer was dried over magnesium sulfate, filtered and evaporated. The residue was purified using column chromatography on silica gel (0-60% ethyl acetate/hexane) providing (R)-3-(2-tert-butyl-5-(1-(2,2-difluorobenzo[d][1,3]-dioxol-5-yl)cyclopropanecarboxamido)-1H-indol-1-yl)-2-hydroxypropyl-4-methyl-benzenesulfonate (3.21 g, 86%). LC/MS (M+1)=641.2. ¹H NMR (400 MHz, CDCl₃) δ 7.77 (d, 2H, J=16 Hz), 7.55 (d, 1H, J=2 Hz), 7.35 (d, 2H, J=16 Hz), 7.31 (m, 3H), 6.96 (s, 1H), 6.94 (dd, 1H, J=2, 8 Hz), 6.22 (s, 1H), 4.33 (m, 1H), 4.31 (dd, 1H, J=6, 15 Hz), 4.28 (dd, 1H, J=11, 15 Hz), 4.18 (m, 1H), 3.40 (dd, 1H, J=3, 6 Hz), 3.36 (dd, 1H, J=3, 6 Hz), 2.46 (s, 3H), 2.40 (br s, 1H), 1.74 (m, 2H), 1.40 (s, 9H), 1.11 (m, 2H).

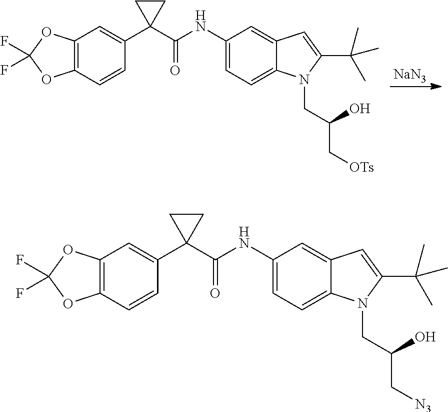 [SMILES: Cc1ccc(S(=O)(=O)OC[C@H](O)Cn2c(C(C)(C)C)cc3cc(NC(=O)C4(c5ccc6c(c5)OC(F)(F)O6)CC4)ccc32)cc1 ; CC(C)(C)c1cc2cc(NC(=O)C3(c4ccc5c(c4)OC(F)(F)O5)CC3)ccc2n1C[C@@H](O)CN=[N+]=[N-] ; "NaN3"]

### (R)—N-(1-(3-Azido-2-hydroxypropyl)-2-tert-butyl-1H-indol-5-yl)-1-(2,2-difluorobenzo[d][1,3]dioxol-5-yl)cyclopropanecarboxamide

To a stirred solution (R)-3-(2-tert-butyl-5-(1-(2,2-difluorobenzo[d][1,3]dioxol-5-yl)cyclopropanecarboxamido)-1H-indol-1-yl)-2-hydroxypropyl-4-methylbenzenesulfonate (3.2 g, 5.0 mmol) in DMF (6 mL) was added sodium azide (2.0 g, 30 mmol). The reaction was heated at 80° C. for 2 h. The mixture was partitioned between 20 mL ethyl acetate and 20 mL water. The layers were separated and the organic layer was evaporated. The residue was purified using column chromatography (0-85% ethyl acetate/hexane) to give (R)—N-(1-(3-azido-2-hydroxypropyl)-2-tert-butyl-1H-indol-5-yl)-1-(2,2-difluorobenzo[d][1,3]dioxol-5-yl)-cyclopropanecarboxamide (2.48 g). LC/MS (M+1)=512.5. ¹H NMR (400 MHz, CDCl₃) δ 7.55 (d, 1H, J=2 Hz), 7.31 (m, 3H), 6.96 (s, 1H), 6.94 (dd, 1H, J=2, 8 Hz), 6.22 (s, 1H), 4.33 (m, 1H), 4.31 (dd, 1H, J=6, 15 Hz), 4.28 (dd, 1H, J=11, 15 Hz), 4.18 (m, 1H), 3.40 (dd, 1H, J=3, 6 Hz), 3.36 (dd, 1H, J=3, 6 Hz), 2.40 (br s, 1H), 1.74 (m, 2H), 1.40 (s, 9H), 1.11 (m, 2H).

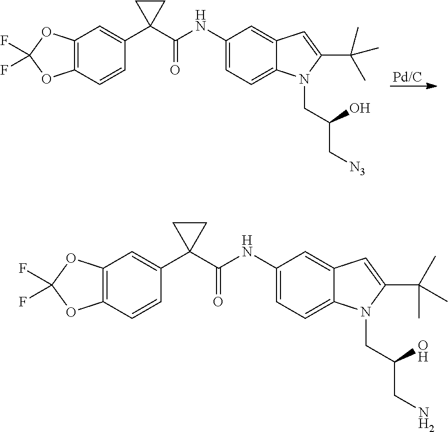

### (S)—N-(1-(3-Amino-2-hydroxypropyl)-2-tert-butyl-1H-indol-5-yl)-1-(2,2-difluoro-benzo[d][1,3]dioxol-5-yl)cyclopropanecarboxamide

To a stirred solution (R)—N-(1-(3-azido-2-hydroxypropyl)-2-tert-butyl-1H-indol-5-yl)-1-(2,2-difluorobenzo[d][1,3]dioxol-5-yl)cyclopropanecarboxamide (2.4 g, 4.0 mmol) in MeOH (25 mL) was added 5% Pd/C (2.4 g) under a Hydrogen gas filled balloon. After 18 h, the reaction mixture was filtered through celite and rinsed with 300 mL ethyl acetate. The organic layer was washed with 1N HCl and evaporated to give (S)—N-(1-(3-amino-2-hydroxypropyl)-2-tert-butyl-1H-indol-5-yl)-1-(2,2-difluoro-benzo[d][1,3]-dioxol-5-yl)cyclopropane-carboxamide (1.37 g). MS (M+1)=486.5.

### Example 99

### (5)-Methyl 3-(2-tert-butyl-5-(1-(2,2-difluorobenzo[d][1,3]dioxol-5-yl)cyclopropanecarboxamido)-1H-indol-1-yl)-2-hydroxypropylcarbamate

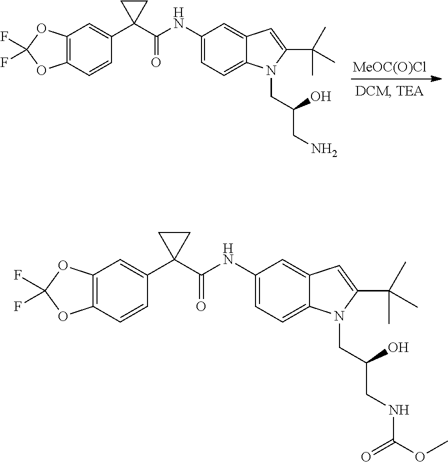 [SMILES: "MeOC(O)Cl" ; CC(C)(C)c1cc2cc(NC(=O)C3(c4ccc5c(c4)OC(F)(F)O5)CC3)ccc2n1C[C@@H](O)CN ; COC(=O)NC[C@H](O)Cn1c(C(C)(C)C)cc2cc(NC(=O)C3(c4ccc5c(c4)OC(F)(F)O5)CC3)ccc21]

To a stirred solution (R)—N-(1-(3-amino-2-hydroxypropyl)-2-tert-butyl-1H-indol-5-yl)-1-(2,2-difluorobenzo[d][1,3]dioxol-5-yl)cyclopropanecarboxamide (0.10 g, 0.20 mmol) in methanol (1 mL) was added 2 drops of triethylamine and methylchloroformyl chloride (0.020 mL, 0.25 mmol). After 30 min, the reaction mixture was filtered and purified using reverse phase HPLC providing (S)-methyl 3-(2-tert-butyl-5-(1-(2,2-difluorobenzo[d][1,3]dioxol-5-yl)cyclo-propanecarboxamido)-1H-indol-1-yl)-2-hydroxypropylcarbamate. The retention time on a three minute run is 1.40 min. LC/MS (M+1)=5443. ¹H NMR (400 MHz, CDCl₃) δ 7.52 (d, 1H, J=2 Hz), 7.30 (dd, 1H, J=2, 8 Hz), 7.28 (m, 1H), 7.22 (d, 1H, J=8 Hz), 7.14 (d, 1H, J=8 Hz), 7.04 (br s, 1H), 6.97 (dd, 1H, J=2, 8 Hz), 6.24 (s, 1H), 5.19 (1H, br s), 4.31 (dd, 1H, J=6, 15 Hz), 4.28 (dd, 1H, J=11, 15 Hz), 4.18 (m, 1H), 3.70 (s, 3H), 3.40 (dd, 1H, J=3, 6 Hz), 3.36 (dd, 1H, J=3, 6 Hz), 3.26 (m, 1H), 1.74 (m, 2H), 1.40 (s, 9H), 1.11 (m, 2H).

### Example 100

### 4-(5-(1-(Benzo[d][1,3]dioxol-5-yl)cyclopropanecarboxamido)-2-tert-butyl-1H-indol-1-yl)butanoic acid

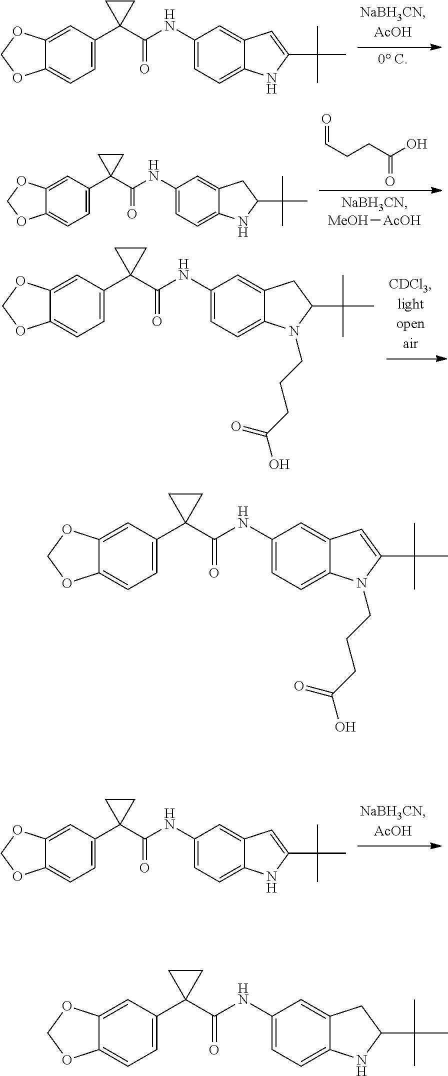

### 1-(Benzo[d][1,3]dioxol-5-yl)-N-(2-tert-butylindolin-5-yl)cyclopropanecarboxamide

To a solution of 1-(benzo[d][1,3]dioxol-5-yl)-N-(2-tert-butyl-1H-indol-5-yl)cyclo-propanecarboxamide (851 mg, 2.26 mmol) in acetic acid (60 mL) was added NaBH₃CN (309 mg, 4.91 mmol) at 0° C. The reaction mixture was stirred for 5 min at room temperature after which no starting material could be detected by LCMS. The solvent was evaporated under reduced pressure and the residue was purified by column chromatography on silica gel (5-40% ethyl acetate/hexanes) to give 1-(benzo[d][1,3]dioxol-5-yl)-N-(2-tert-butylindolin-5-yl)cyclopropanecarboxamide (760 mg, 89%).

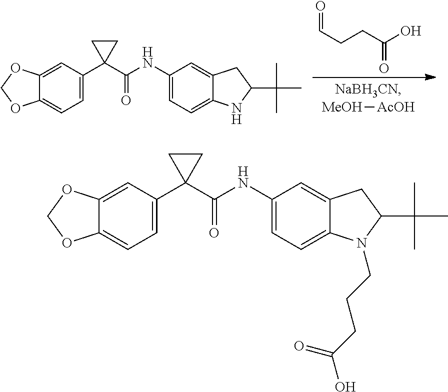

### 4-(5-(1-(Benzo[d][1,3]dioxol-5-yl)cyclopropanecarboxamido)-2-tert-butylindolin-1-yl)butanoic acid

To a solution of 1-(benzo[d][1,3]dioxol-5-yl)-N-(2-tert-butylindolin-5-yl)cyclopropanecarboxamide (350 mg, 0.93 mmol, 1 eq) in anhydrous methanol (6.5 mL) and AcOH (65 μL) was added 4-oxobutanoic acid (15% in water, 710 mg, 1.0 mmol) at room temperature. After 20 min of stirring, NaBH₃CN (130 mg, 2.0 mmol) was added in one portion and the reaction mixture was stirred for another 4 h at room temperature. The reaction mixture was quenched by addition of AcOH (0.5 mL) at 0° C. and the solvent was removed under reduced pressure. The residue was purified by column chromatography on silica gel (5-75% ethyl acetate/hexanes) to give 4-(5-(1-(benzo[d][1,3]dioxol-5-yl)cyclopropanecarboxamido)-2-tert-butylindolin-1-yl)butanoic acid (130 ma. 30%).

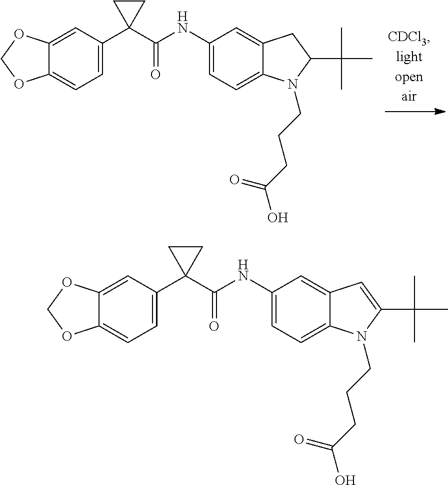 [SMILES: CC(C)(C)C1Cc2cc(NC(=O)C3(c4ccc5c(c4)OCO5)CC3)ccc2N1CCCC(=O)O ; CC(C)(C)c1cc2cc(NC(=O)C3(c4ccc5c(c4)OCO5)CC3)ccc2n1CCCC(=O)O]

### 4-(5-(1-(Benzo[d][1,3]dioxol-5-yl)cyclopropanecarboxamido)-2-tert-butyl-1H-indol-1-yl)butanoic acid

4-(5-(1-(Benzo[d][1,3]dioxol-5-yl)cyclopropanecarboxamido)-2-tert-butylindolin-1-yl)butanoic acid (130 mg, 0.28 mmol) was taken up in a mixture of acetonitrile-H₂O-TFA. The solvent was removed under reduced pressure and the residue obtained was dissolved in CDCl₃. After a brief exposition to daylight (5-10 min), the solution turned purple. The mixture was stirred open to the atmosphere at room temperature until complete disappearance of the starting material (8 h). Solvent was removed under reduced pressure and the residue was purified by reverse phase HPLC to give 4-(5-(1-(benzo[d][1,3]dioxol-5-yl)cyclopropanecarboxamido)-2-tert-butyl-1H-indol-1-yl)butanoic acid. ¹H NMR (400 MHz, CDCl₃) δ 7.52 (d, J=1.9 Hz, 1H), 7.18 (d, J=2.1 Hz, 1H), 7.16 (s, 1H), 7.03 (dd, J=9.4, 1.9 Hz, 1H), 7.00-6.98 (m, 2H), 6.85 (d, J=7.9 Hz, 1H), 6.16 (s, 1H), 6.02 (s, 2H), 4.29-4.24 (m, 2H), 2.48 (dd, J=6.9, 6.9 Hz, 2H), 2.12-2.04 (m, 2H), 1.69 (dd, J=6.8, 3.7 Hz, 2H), 1.43 (s, 9H), 1.09 (dd, J=6.8, 3.7 Hz, 2H). MS (ESI) m/e (M+H⁺) 463.0.

### Example 101

### 1-(Benzo[d][1,3]dioxol-5-yl)-N-(2-tert-butyl-1-(4-(2-hydroxyethyl-amino)-4-oxobutyl)-1H-indol-5-yl)cyclopropanecarboxamide

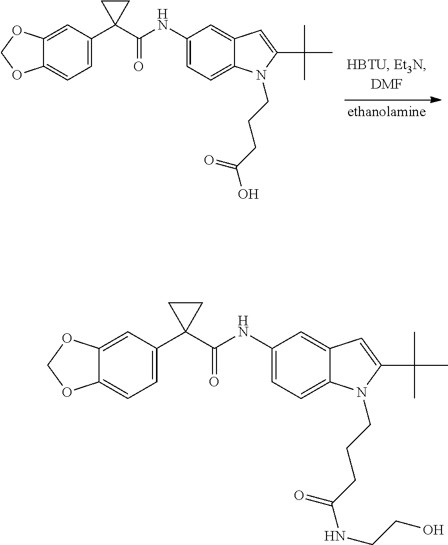 [SMILES: CC(C)(C)c1cc2cc(NC(=O)C3(c4ccc5c(c4)OCO5)CC3)ccc2n1CCCC(=O)O ; CC(C)(C)c1cc2cc(NC(=O)C3(c4ccc5c(c4)OCO5)CC3)ccc2n1CCCC(=O)NCCO]

To a solution of 4-(5-(1-(benzo[d][1,3]dioxol-5-yl)cyclopropanecarboxamido)-2-tert-butyl-1H-indol-1-yl)butanoic acid (10 mg) in anhydrous DMF (0.25 mL) were successively added Et₃N (9.5 mL, 0.069 mmol) and HBTU (8.2 mg, 0.022 mmol). After stirring for 10 min at 60° C., ethanolamine (1.3 μL, 0.022 mmol) was added, and the mixture was stirred for another 4 h at 60° C. 1-(Benzo[d][1,3]dioxol-5-yl)-N-(2-tert-butyl-1-(4-(2-hydroxyethyl-amino)-4-oxobutyl)-1H-indol-5-yl)cyclopropanecarboxamide (5.8 mg, 64%) was obtained after purification by preparative HPLC. MS (ESI) m/e (M+H⁺) 506.0.

### Example 102

### 1-(Benzo[d][1,3]dioxol-5-yl)-N-(2-tert-butyl-1-(2-(dimethylamino)-2-oxoethyl)-1H-indol-5-yl)cyclopropanecarboxamide

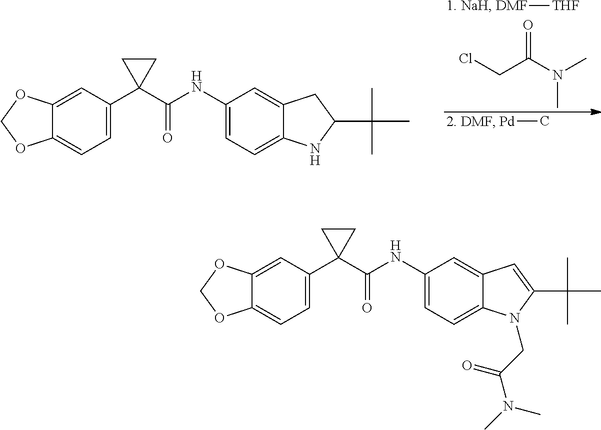

To a solution of 1-(benzo[d][1,3]dioxol-5-yl)-N-(2-tert-butylindolin-5-yl)cyclopropanecarboxamide (62 mg, 0.16 mmol) in anhydrous DMF (0.11 mL) and THF (1 mL) was added NaH (60% in mineral oil, 21 mg, 0.51 mmol) at room temperature under N₂. After 30 min of stirring, the reaction mixture was cooled to 0° C. and 2-chloro-N,N-dimethylacetamide (11 mL, 0.14 mmol,) was added. The reaction mixture was stirred for 5 min at 0° C. and then for 10 h at room temperature. The mixture was purified by preparative HPLC and the resultant solid was dissolved in DMF (0.6 mL) in the presence of Pd—C (10 mg). The mixture was stirred open to the atmosphere overnight at room temperature. The reaction mixture was filtrated and purified by preparative HPLC providing 1-(benzo[d][1,3]dioxol-5-yl)-N-(2-tert-butyl-1-(2-(dimethylamino)-2-oxoethyl)-1H-indol-5-yl)cyclopropanecarboxamide. MS (ESI) m/e (M+H⁺) 462.0.

### Example 103

### 3-(2-tert-Butyl-5-(1-(2,2-difluorobenzo[d][1,3]dioxol-5-yl)cyclo-propanecarboxamido)-1H-indol-1-yl)propanoic acid

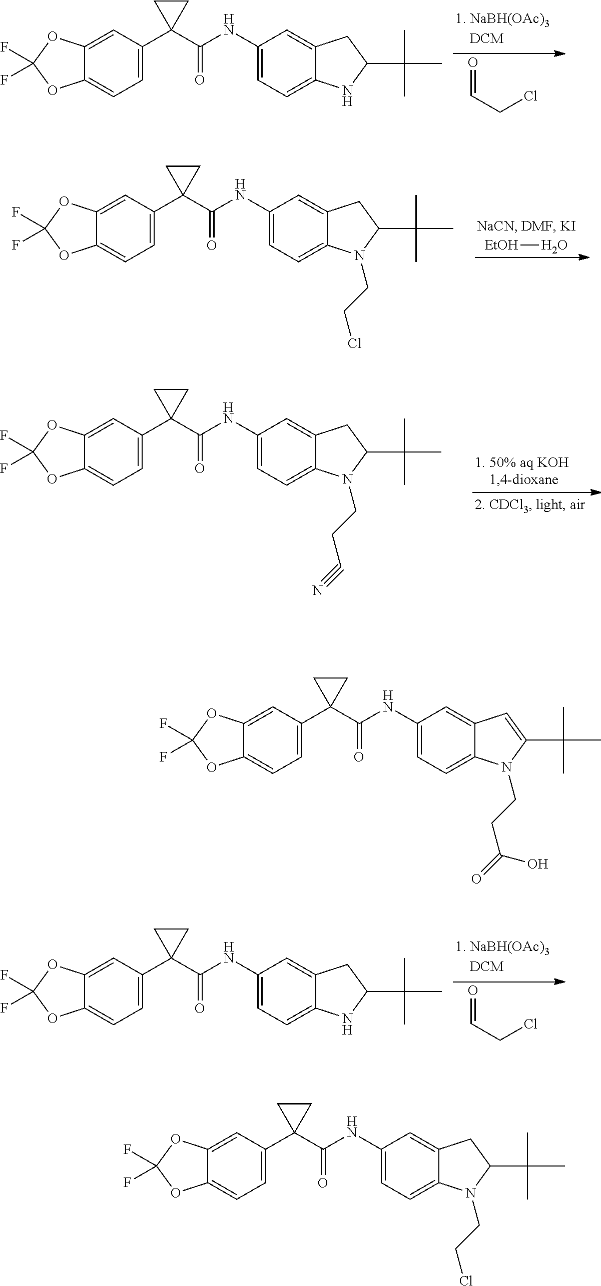

### N-(2-tert-Butyl-1-(2-chloroethyl)indolin-5-yl)-1-(2,2-difluorobenzo[d][1,3]dioxol-5-yl)cyclopropanecarboxamide

To a solution of N-(2-tert-butyl-1-(2-cyanoethyl)indolin-5-yl)-1-(2,2-difluorobenzo[d][1,3]dioxol-5-yl)cyclopropanecarboxamide (71 mg, 0.17 mmol) in anhydrous dichloromethane (1 mL) was added chloroacetaldehyde (53 μL, 0.41 mmol) at room temperature under N₂. After 20 min of stirring, NaBH(OAc)₃ (90 mg, 0.42 mmol) was added in two portions. The reaction mixture was stirred overnight at room temperature. The product was purified by column chromatography on silica gel (2-15% ethyl acetate/hexanes) providing N-(2-tert-butyl-1-(2-chloroethyl)indolin-5-yl)-1-(2,2-difluorobenzo[d][1,3]dioxol-5-yl)cyclopropanecarboxamide (51 mg. 63%).

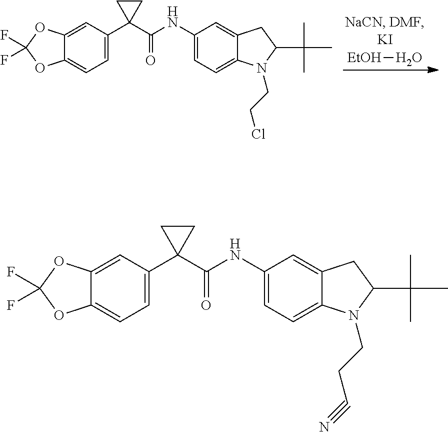

### N-(2-tert-Butyl-1-(2-cyanoethyl)indolin-5-yl)-1-(2,2-difluorobenzo[d][1,3]dioxol-5-yl)cyclopropanecarboxamide

N-(2-tert-butyl-1-(2-chloroethyl)indolin-5-yl)-1-(2,2-difluorobenzo[d][1,3]dioxol-5-yl)cyclopropanecarboxamide (51 mg), NaCN (16 mg, 0.32 mmol) and KI (cat) in EtOH (0.6 mL) and water (0.3 mL) were combined and heated at 110° C. for 30 min in the microwave. The solvent was removed under reduced pressure and the residue was purified by column chromatography on silica gel (2-15% ethyl acetate/hexanes) providing N-(2-tert-butyl-1-(2-cyanoethyl)indolin-5-yl)-1-(2,2-difluorobenzo[d][1,3]dioxol-5-yl)cyclopropanecarboxamide (24 mg, 48%).

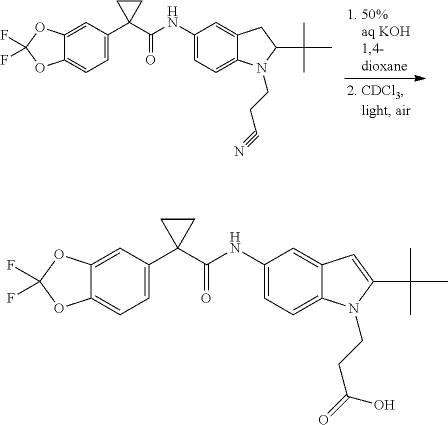 [SMILES: CC(C)(C)C1Cc2cc(NC(=O)C3(c4ccc5c(c4)OC(F)(F)O5)CC3)ccc2N1CCC#N ; CC(C)(C)c1cc2cc(NC(=O)C3(c4ccc5c(c4)OC(F)(F)O5)CC3)ccc2n1CCC(=O)O]

### 3-(2-tert-Butyl-5-(1-(2,2-difluorobenzo[d][1,3]dioxol-5-yl)cyclo-propanecarbox-amido)-1H-indol-1-yl)propanoic acid

N-(2-tert-butyl-1-(2-cyanoethyl)indolin-5-yl)-1-(2,2-difluorobenzo[d][1,3]dioxol-5-yl)cyclopropane-carboxamide (24 mg, 0.050 mmol) was taken up in 50% aq. KOH (0.5 mL) and 1,4-dioxane (1 mL). The mixture was heated at 125° C. for 2 h. The solvent was removed and the residue was purified by preparative HPLC. The residue was dissolved in CDCl₃ (1 mL) then briefly exposed to daylight. The purple solution that formed was stirred until complete disappearance of the starting material (1 h). The solvent was removed under reduced pressure and the residue was purified by preparative HPLC providing 3-(2-tert-butyl-5-(1-(2,2-difluorobenzo[d][1,3]dioxol-5-yl)cyclo-propanecarboxamido)-1H-indol-1-yl)propanoic acid. MS (ESI) m/e (M+H⁺) 485.0.

### Example 104

### 1-(Benzo[d][1,3]dioxol-5-yl)-N-(2-tert-butyl-6-fluoro-1-(2-hydroxy-ethyl)-1H-indol-5-yl)cyclopropenecarboxamide

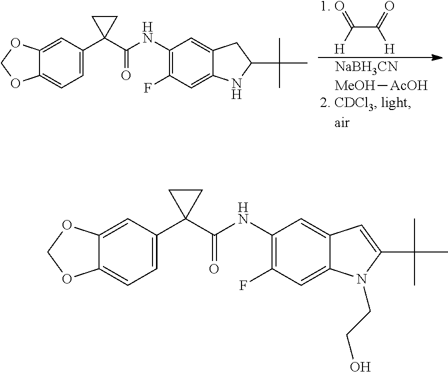

To a solution of 1-(benzo[d][1,3]dioxol-5-yl)-N-(2-tert-butyl-6-fluoroindolin-5-yl)cyclopropanecarboxamide (340 mg, 0.86 mmol) in anhydrous MeOH (5.7 mL) containing 1% of acetic acid was added glyoxal 40% in water (0.60 mL, 5.2 mmol) at room temperature under N₂. After 20 min of stirring, NaBH₃CN (120 mg, 1.9 mmol) was added in one portion and the reaction mixture was stirred overnight at room temperature. The solvent was removed under reduced pressure and the residue obtained was purified by column chromatography on silica gel (10-40% ethyl acetate/hexanes) providing a pale yellow oil which was treated with 50/50 CH₃CN—H₂O containing 0.05% TFA and CDCl₃. Solvent was removed under reduced pressure and the residue was purified by column chromatography on silica gel (20-35% ethyl acetate/hexanes) to give 1-(benzo[d][1,3]dioxol-5-yl)-N-(2-tert-butyl-6-fluoro-1-(2-hydroxyethyl)-1H-indol-5-yl)cyclopropanecarboxamide. ¹H NMR (400 MHz, CDCl₃) 8.02 (d, J=7.7 Hz, 1H), 7.30 (d, J=2.1 Hz, 1H), 6.93 (dd, J=1.6, 7.9 Hz, 1H), 6.90 (d, J=1.6 Hz, 1H), 6.90 (d, J=1.6 Hz, 1H), 6.78 (d, J=7.9 Hz, 1H), 6.08 (s, 1H), 5.92 (s, 2H), 4.21 (dd, J=6.9, 6.9 Hz, 2H), 3.68 (m, 2H), 2.28 (s, 1H), 1.60 (dd, J=3.7, 6.7 Hz, 2H), 1.35-1.32 (m, 9H), 1.04 (dd, J=3.7, 6.8 Hz, 2H). MS (ESI) m/e (M+H⁺) 439.0.

### Example 105

### 1-(Benzo[d][1,3]dioxol-5-yl)-N-(2-tert-butyl-6-fluoro-1-(3-hydroxy-propyl)-1H-indol-5-yl)cyclopropanecarboxamide

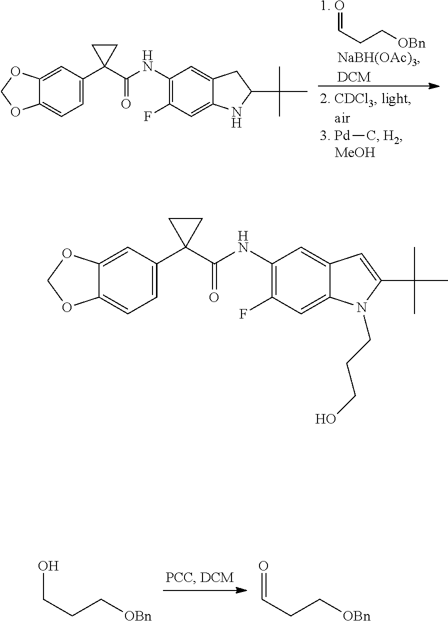

### 3-(Benzyloxy)propanal

To a suspension of PCC (606 mg, 2.82 mmol) in anhydrous dichloromethane (8 mL) at room temperature under N₂ was added a solution of 3-benzyloxy-1-propanol (310 mg, 1.88 mmol) in anhydrous dichloromethane. The reaction mixture was stirred overnight at room temperature, filtrated through Celite, and concentrated. The residue was purified by column chromatography on silica gel (1-10% ethyl acetate/hexanes) to give 3-(benzyloxy)propanal (243 mg, 79%).

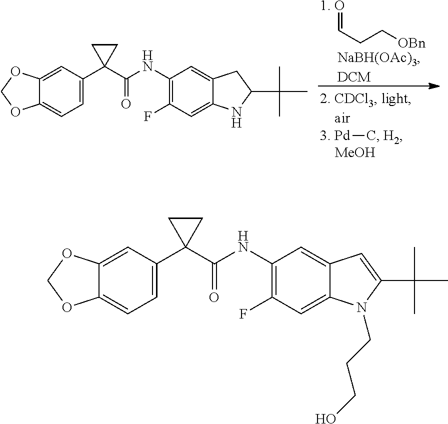

### 1-(Benzo[d][1,3]dioxol-5-yl)-N-(2-tert-butyl-6-fluoro-1-(3-hydroxypropyl)-1H-indol-5-yl)cyclopropanecarboxamide

To a solution of 1-(benzo[d][1,3]dioxol-5-yl)-N-(2-tert-butyl-6-fluoroindolin-5-yl)cyclopropanecarboxamide (160 mg, 0.50 mmol) in anhydrous dichloromethane (3.4 mL) was added 3-(benzyloxy)propanal (160 mg, 0.98 mmol) at room temperature. After 10 min of stirring, NaBH(OAc)₃ (140 mg, 0.65 mmol) was added in one portion and the reaction mixture was stirred for 4 h at room temperature. The solvent was removed under reduced pressure and the residue was taken-up in a mixture of 50/50 CH₃CN—H₂O containing 0.05% TFA. The mixture was concentrated to dryness and the residue was dissolved in CDCl₃ (5 mL) and briefly exposed to daylight. The purple solution was stirred open to the atmosphere at room temperature for 2 h. The solvent was removed under reduced pressure and the residue was treated with Pd—C (10 mg) in MeOH (2 mL) under 1 atm of H₂ for 2 h. The catalyst was filtered through Celite and the solvent was removed under reduced pressure. The residue was purified by preparative TLC 30% ethyl acetate/hexanes to provide 1-(benzo[d][1,3]dioxol-5-yl)-N-(2-tert-butyl-6-fluoro-1-(3-hydroxypropyl)-1H-indol-5-yl)cyclopropanecarboxamide (18 mg, 8% from 1-(benzo[d][1,3]dioxol-5-yl)-N-(2-tert-butyl-6-fluoroindolin-5-yl)cyclopropane-carboxamide). ¹H NMR (400 MHz, CDCl₃) δ 8.11 (d, J=7.8 Hz, 1H), 7.31 (d, J=2.2 Hz, 1H), 6.94 (dd, J=7.9, 1.7 Hz, 1H), 6.91 (d, J=1.6 Hz, 1H), 6.85 (d, J=11.7 Hz, 1H), 6.79 (d, J=7.9 Hz, 1H), 6.10 (s, 1H), 5.94 (s, 2H), 4.25-4.21 (m, 2H), 3.70 (dd, J=5.7, 5.7 Hz, 2H), 1.93-1.86 (m, 2H), 1.61 (dd, J=6.8, 3.7 Hz, 2H), 1.35 (s, 9H), 1.04 (dd, J=6.8, 3.7 Hz, 2H). MS (ESI) m/e (M+H⁺) 453.0.

### Example 106

### N-(1-(2-Acetamidoethyl)-2-tert-butyl-1H-indol-5-yl)-1-(benzo[d][1,3]-dioxol-5-yl)cyclopropanecarboxamide

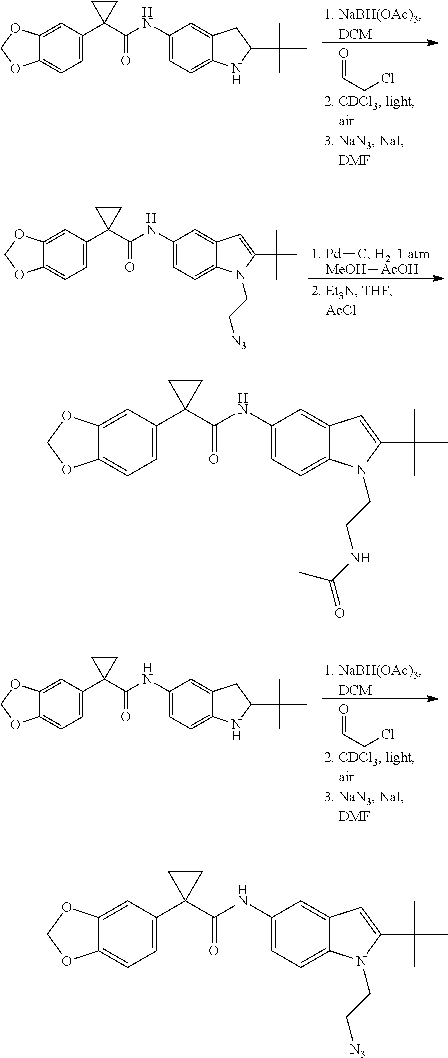

### N-(1-(2-azidoethyl)-2-tert-butyl-1H-indol-5-yl)-1-(benzo[d][1,3]dioxol-5-yl)-cyclopropanecarboxamide

To a solution of 1-(benzo[d][1,3]dioxol-5-yl)-N-(2-tert-butylindolin-5-yl)cyclopropane-carboxamide (73 mg, 0.19 mmol) in anhydrous dichloromethane (1.2 mL) was added chloroacetaldehyde (60 μL, 0.24 mmol) at room temperature. After 10 min of stirring, NaBH(OAc)₃ (52 mg, 0.24 mmol) was added in one portion and the reaction mixture was stirred for another 30 min at room temperature. The solvent was removed under reduced pressure and the residue was purified by preparative HPLC to give the indoline, which oxidized to the corresponding indole when taken-up in CDCl₃. The resulting indole was treated with NaN₃ (58 mg, 0.89 mmol) and NaI (cat) in anhydrous DMF (0.8 mL) for 2 h at 85° C. The reaction mixture was purified by preparative HPLC providing N-(1-(2-azidoethyl)-2-tert-butyl-1H-indol-5-yl)-1-(benzo[d][1,3]dioxol-5-yl)cyclopropanecarboxamide (15 mg, 18% from 1-(benzo[d][1,3]dioxol-5-yl)-N-(2-tert-butylindolin-5-yl)cyclopropane-carboxamide).

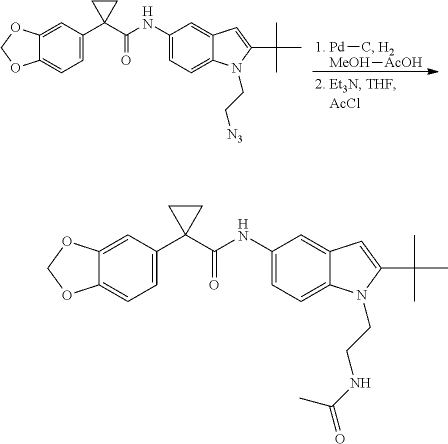

### N-(1-(2-Acetamidoethyl)-2-tert-butyl-1H-indol-5-yl)-1-(benzo[d][1,3]-dioxol-5-yl)cyclopropanecarboxamide

A solution of N-(1-(2-azidoethyl)-2-tert-butyl-1H-indol-5-yl)-1-(benzo[d][1,3]dioxol-5-yl)cyclopropanecarboxamide (13 mg, 0.029 mmol) in MeOH—AcOH (0.2 mL, 99:1) in the presence of Pd—C (2 mg) was stirred at room temperature under 1 atm of H₂ for 2 h, filtered through Celite, and concentrated under reduced pressure. The crude product was treated with AcCl (0.05 mL) and Et₃N (0.05 mL) in anhydrous THF (0.2 mL) at 0° C. for 30 min and then 1 h at room temperature. The mixture was purified by preparative HPLC providing N-(1-(2-acetamidoethyl)-2-tert-butyl-1H-indol-5-yl)-1-(benzo[d][1,3]-dioxol-5-yl)cyclopropanecarboxamide. MS (ESI) m/e (M+H⁺) 462.0.

### Example 107

### N-(2-tert-Butyl-1-(3-cyano-2-hydroxypropyl)-1H-indol-5-yl)-1-(2,2-difluorobenzo[d][1,3]dioxol-5-yl)cyclopropanecarboxamide

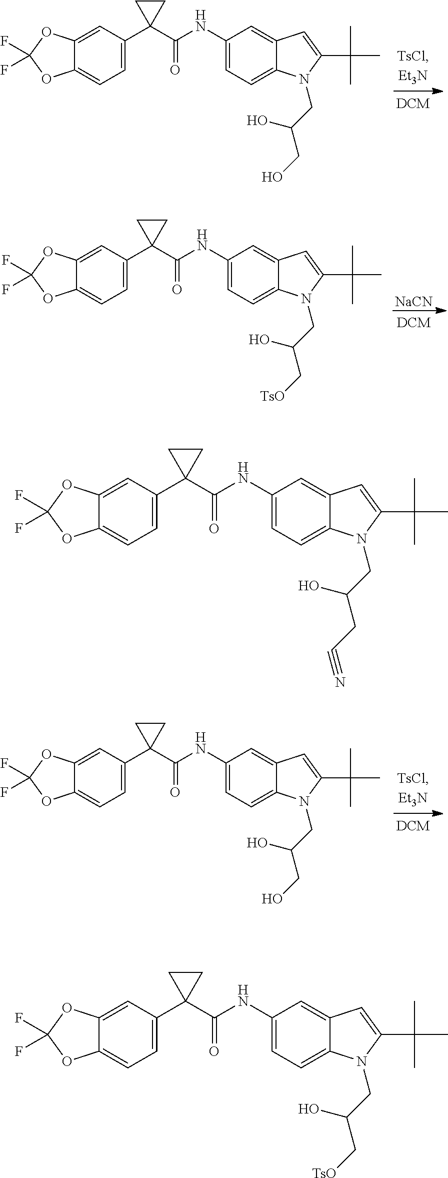

### 3-(2-tert-Butyl-5-(1-(2,2-difluorobenzo[d][1,3]dioxol-5-yl)cyclopropanecarbox-amido)-1H-indol-1-yl)-2-hydroxypropyl-4-methylbenzenesulfonate

To a solution of N-(2-tert-butyl-1-(2,3-dihydroxypropyl)-1H-indol-5-yl)-1-(2,2-difluorobenzo[d][1,3]-dioxol-5-yl)cyclopropanecarboxamide (172 mg, 0.35 mmol) in anhydrous dichloromethane (1.4 mL) at 0° C. in the presence of Et₃N (56 μL, 0.40 mmol) was added TsCl (71 mg, 0.37 mmol). The reaction mixture was stirred for 2 h at room temperature before being cooled to 0° C. and another portion of TsCl (71 mg, 0.37 mmol) was added. After 1 h of stirring at room temperature, the mixture was purified by column chromatography on silica gel (10-30% ethyl acetate/hexanes) providing 3-(2-tert-butyl-5-(1-(2,2-difluorobenzo[d][1,3]dioxol-5-yl)cyclopropanecarboxamido)-1H-indol-1-yl)-2-hydroxypropyl-4-methylbenzene-sulfonate (146 mg, 64%).

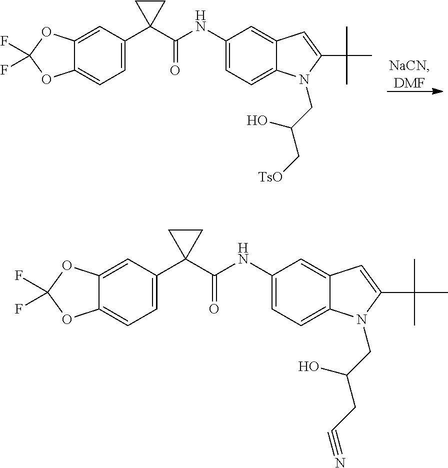

### N-(2-tert-Butyl-1-(3-cyano-2-hydroxypropyl)-1H-indol-5-yl)-1-(2,2-difluorobenzo[d][1,3]dioxol-5-yl)cyclopropanecarboxamide

N-(2-tert-Butyl-1-(3-cyano-2-hydroxypropyl)-1H-indol-5-yl)-1-(2,2-difluorobenzo[d][1,3]dioxol-5-yl)-cyclopropanecarboxamide (145 mg, 0.226 mmol) was treated with powdered NaCN (34 mg, 0.69 mmol) in anhydrous DMF (1.5 mL) at 85° C. for 2 h. The reaction mixture was cooled down to room temperature before it was diluted with dichloromethane (10 mL) and aq. sat. NaHCO₃ (10 mL). The organic phase was separated and the aqueous phase was extracted with dichloromethane (2×10 mL). The organic phases were combined, washed with brine, dried with sodium sulfate, filtered then concentrated. The residue was purified by column chromatography on silica gel (25-55% ethyl acetate/hexanes) providing N-(2-tert-butyl-1-(3-cyano-2-hydroxypropyl)-1H-indol-5-yl)-1-(2,2-difluorobenzo[d][1,3]dioxol-5-yl)cyclopropanecarboxamide (89 mg, 79%). ¹H NMR (400 MHz, CDCl₃) δ 7.43 (d, J=1.9 Hz, 1H), 7.20-7.16 (m, 2H), 7.08 (d, J=8.8 Hz, 1H), 7.04 (d, J=8.2 Hz, 1H), 6.94 (s, 1H), 6.88 (dd, J=8.7, 2.0 Hz, 1H), 6.16 (s, 1H), 4.32-4.19 (m, 3H), 2.83 (s, 1H), 2.40 (dd, J=5.2, 5.2 Hz, 2H), 1.62 (dd, J=6.6, 3.6 Hz, 2H), 1.35 (s, 9H), 1.04 (dd, J=6.9, 3.9 Hz, 2H). MS (ESI) m/e (M+H⁺) 496.0.

### Example 108

### N-(2-tert-Butyl-1-(2-hydroxy-3-(2H-tetrazol-5-yl)propyl)4H-indol-5-yl)-1-(2,2-difluorobenzo[d][1,3]dioxol-5-yl)cyclopropanecarboxamide

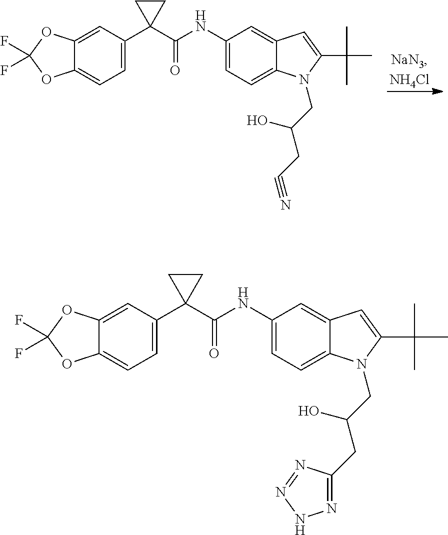

To a solution of N-(2-tert-butyl-1-(3-cyano-2-hydroxypropyl)-1H-indol-5-yl)-1-(2,2-difluorobenzo[d][1,3]dioxol-5-yl)cyclopropanecarboxamide (27 mg, 0.054 mmol) in anhydrous DMF (1.2 mL) were successively added NH₄Cl (35 mg, 0.65 mmol) and NaN₃ (43 mg, 0.65 mmol) at room temperature. The reaction mixture was stirred for 4 h at 110° C. in the microwave, at which stage 50% of the starting material was converted to the desired product. The reaction mixture was purified by preparative HPLC to provide N-(2-tert-butyl-1-(2-hydroxy-3-(2H-tetrazol-5-yl)propyl)-1H-indol-5-yl)-1-(2,2-difluorobenzo-[d][1,3]dioxol-5-yl)cyclopropanecarboxamide. MS (ESI) m/e (M+H⁺) 539.0.

### Example 109

### 4-(2-tert-Butyl-5-(1-(2,2-difluorobenzo[d][1,3]dioxol-5-yl)cyclo-propanecarboxamido)-1H-indol-1-yl)-3-hydroxybutanoic acid

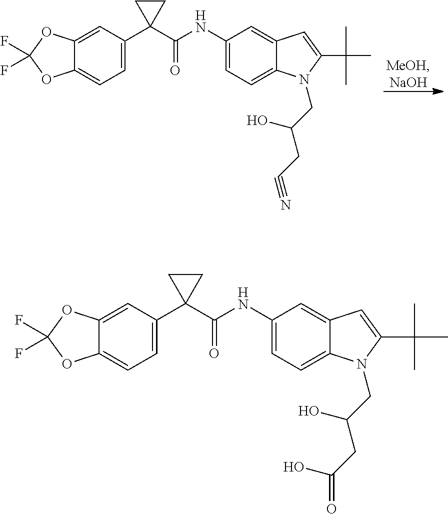

A solution of N-(2-tert-butyl-1-(3-cyano-2-hydroxypropyl)-1H-indol-5-yl)-1-(2,2-difluorobenzo[d][1,3]dioxol-5-yl)cyclopropanecarboxamide (14 mg, 0.028 mmol) in methanol (0.8 mL) and 4 M NaOH (0.8 mL) was stirred at 60° C. for 4 h. The reaction mixture was neutralized with 4 M HCl and concentrated. The residue was purified by preparative HPLC to provide 4-(2-tert-butyl-5-(1-(2,2-difluorobenzo[d][1,3]dioxol-5-yl)cyclopropanecarboxamido)-1H-indol-1-yl)-3-hydroxybutanoic acid. MS (ESI) m/e (M+H⁴) 515.0.

### Example 110

### N-(1-(2-(2H-Tetrazol-5-yl)ethyl)-2-tert-butyl-1H-indol-5-yl)-1-(benzo[d][1,3]dioxol-5-yl)cyclopropanecarboxamide

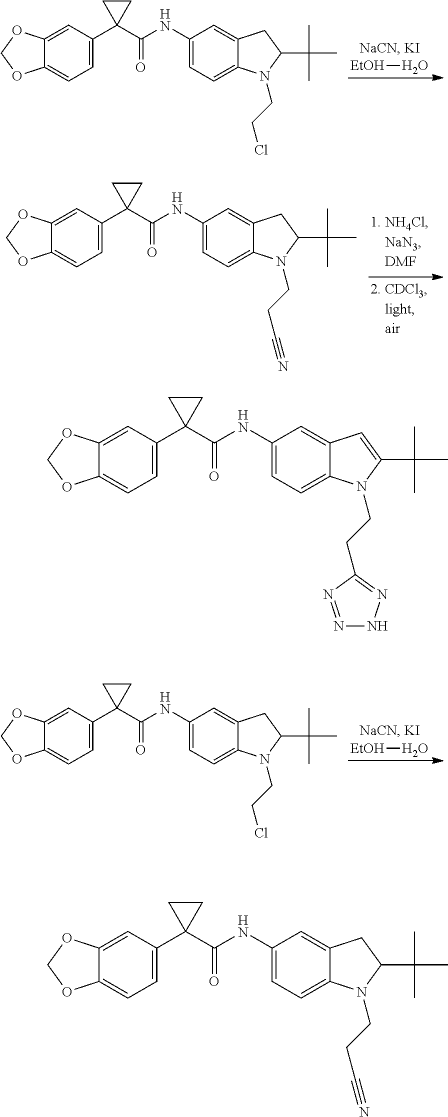

### 1-(Benzo[d][1,3]dioxol-5-yl)-N-(2-tert-butyl-1-(2-cyanoethyl)indolin-5-yl)-cyclopropanecarboxamide

To a solution of 1-(benzo[d][1,3]dioxol-5-yl)-N-(2-tert-butyl-1-(2-chloroethyl)indolin-5-yl)cyclopropanecarboxamide (66 mg, 0.15 mmol) in ethanol (0.8 mL) and water (0.4 mL) were added NaCN (22 mg, 0.45 mmol) and KI (cat) at room temperature. The reaction mixture was stirred for 30 min at 110° C. in the microwave before being purified by column chromatography on silica gel (5-15% ethyl acetate/hexanes) to provide 1-(benzo[d][1,3]dioxol-5-yl)-N-(2-tert-butyl-1-(2-cyano-ethyl)indolin-5-yl)cyclopropanecarboxamide (50 mg, 77%).

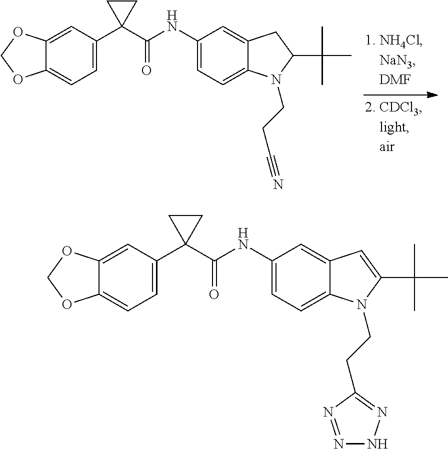 [SMILES: CC(C)(C)C1Cc2cc(NC(=O)C3(c4ccc5c(c4)OCO5)CC3)ccc2N1CCC#N ; CC(C)(C)c1cc2cc(NC(=O)C3(c4ccc5c(c4)OCO5)CC3)ccc2n1CCc1nn[nH]n1]

### N-(1-(2-(2H-Tetrazol-5-yl)ethyl)-2-tert-butyl-1H-indol-5-yl)-1-(benzo[d][1,3]dioxol-5-yl)cyclopropanecarboxamide

To a solution of 1-(benzo[d][1,3]dioxol-5-yl)-N-(2-tert-butyl-1-(2-cyano-ethyl)indolin-5-yl)cyclopropanecarboxamide (50 mg, 0.12 mmol) in anhydrous DMF (2.6 mL) was added NH₄Cl (230 mg, 4.3 mmol) and NaN₃ (280 mg, 4.3 mmol). The reaction mixture was stirred for 30 min at 110° C. in the microwave, filtrated, and purified by preparative HPLC. The solid residue was dissolved in CDCl₃ (3 mL) and briefly (2 to 4 min) exposed to daylight, which initiated a color change (purple). After 2 h of stirring open to the atmosphere at room temperature, the solvent was removed and the residue was purified by preparative HPLC to give N-(1-(2-(2H-tetrazol-5-yl)ethyl)-2-tert-butyl-1H-indol-5-yl)-1-(benzo[d][1,3]dioxol-5-yl)cyclopropanecarboxamide. MS (ESI) m/e (M+H⁺) 473.0.

### Example 111

### 1-(Benzo[d][1,3]dioxol-5-yl)-N-(2-tert-butyl-6-fluoro-1-((tetrahydro-2H-pyran-3-yl)methyl)-1H-indol-5-yl)cyclopropanecarboxamide

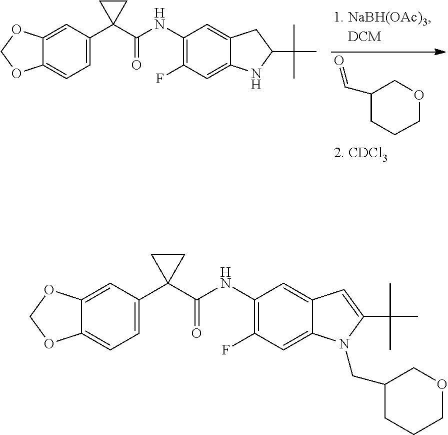

To a solution of 1-(benzo[d][1,3]dioxol-5-yl)-N-(2-tert-butyl-6-fluoroindolin-5-yl)cyclopropane-carboxamide (150 mg, 0.38 mmol) in anhydrous dichloromethane (2.3 mL) at room temperature under N₂ was added tetrahydropyran-3-carbaldehyde (54 mg, 0.47 mmol). After 20 min of stirring, NaBH(OAc)₃ (110 mg, 0.51 mmol) was added in one portion at room temperature. The reaction mixture was stirred for 6 h at room temperature before being purified by column chromatography on silica gel (5-20% ethyl acetate/hexanes) to provide 1-(benzo[d][1,3]dioxol-5-yl)-N-(2-tert-butyl-6-fluoro-1-((tetrahydro-2H-pyran-3-yl)methyl)indolin-5-yl)cyclopropanecarboxamide (95 mg, 50%). CDCl₃ was added to the indoline and the solution was allowed to stir overnight at ambient temperature. The solution was concentrated to give 1-(benzo[d][1,3]dioxol-5-yl)-N-(2-tert-butyl-6-fluoro-1-((tetrahydro-2H-pyran-3-yl)methyl)-1H-indol-5-yl)cyclopropanecarboxamide. MS (ESI) m/e (M+H⁺) 493.0.

### Example 112

### 1-(Benzo[d][1,3]dioxol-5-yl)-N-(2-(2-hydroxypropan-2-yl)-1H-indol-5-yl)cyclopropanecarboxamide

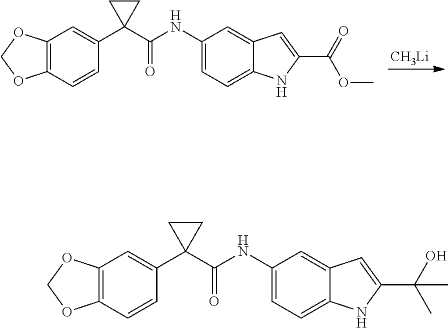

Methyl 5-(1-(benzo[d][1,3]dioxol-5-yl)cyclopropane-carboxamido)-1H-indole-2-carboxylate (100 mg, 0.255 mmol) was dissolved in anhydrous tetrahydrofuran (2 mL) under an argon atmosphere. The solution was cooled to 0° C. in an ice water bath before methyllithium (0.85 mL, 1.6 M in diethyl ether) was added by syringe. The mixture was allowed to warm to room temperature. The crude product was then partitioned between a saturated aqueous solution of sodium chloride (5 mL) and dichloromethane (5 mL). The organic layers were combined, dried over sodium sulfate, filtered, evaporated to dryness, and purified on 12 g of silica gel utilizing a gradient of 20-80% ethyl acetate in hexanes to yield 1-(benzo[d][1,3]dioxol-5-yl)-N-(2-(2-hydroxypropan-2-yl)-1H-indol-5-yl)cyclopropanecarboxamide (35 mg, 36%) as a white solid. ESI-MS m/z calc. 378.2. found 379.1 (M+1)⁺. Retention time of 2.18 minutes. ¹H NMR (400 MHz, DMSO-d6) 10.78 (s, 1H), 8.39 (s, 1H), 7.57 (d, J=1.7 Hz, 1H), 7.17 (d, J=8.6 Hz, 1H), 7.03-6.90 (m, 4H), 6.12 (d, J=1.5 Hz, 1H), 6.03 (s, 2H), 5.18 (s, 1H), 1.50 (s, 6H), 1.41-1.38 (m, 2H), 1.05-0.97 (m, 2H).

### Example 113

### N-(2-(1-Amino-2-methylpropan-2-yl)-1H-indol-5-yl)-1-(benzo[d][1,3]-dioxol-5-yl)cyclopropanecarboxamide

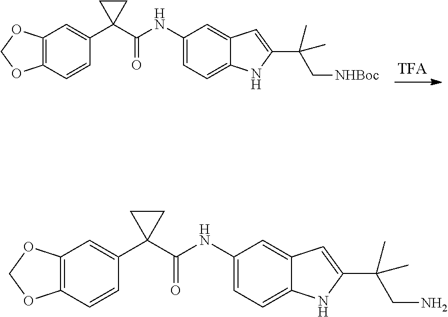 [SMILES: CC(C)(C)OC(=O)NCC(C)(C)c1cc2cc(NC(=O)C3(c4ccc5c(c4)OCO5)CC3)ccc2[nH]1 ; CC(C)(CN)c1cc2cc(NC(=O)C3(c4ccc5c(c4)OCO5)CC3)ccc2[nH]1]

Trifluoroacetic acid (0.75 mL) was added to a solution of tert-butyl 2-(5-(1-(benzo[d][1,3]dioxol-5-yl)cyclopropanecarboxamido)-1H-indol-2-yl)-2-methylpropylcarbamate (77 mg, 0.16 mmol) in dichloromethane (3 mL) and the mixture was stirred at room temperature for 1.5 h. The mixture was evaporated, dissolved in dichloromethane, washed with saturated sodium bicarbonate solution, dried over magnesium sulfate and evaporated to dryness to give N-(2-(1-amino-2-methylpropan-2-yl)-1H-indol-5-yl)-1-(benzo[d][1,3]dioxol-5-yl)cyclopropanecarboxamide (53 mg, 86%). ¹H NMR (400 MHz, CDCl₃) 9.58 (s, 1H), 7.60 (d, J=1.6 Hz, 1H), 7.18-7.15 (m, 2H), 7.02-6.94 (m, 3H), 6.85 (d, J=7.8 Hz, 1H), 6.14 (d, J=1.2 Hz, 1H), 6.02 (s, 2H), 2.84 (s, 2H), 1.68 (dd, J=3.6, 6.7 Hz, 2H), 1.32 (s, 6H), 1.08 (dd, J=3.7, 6.8 Hz, 2H).

### Example 114

### 1-(Benzo[d][1,3]dioxol-5-yl)-N-(2-(1-(dimethylamino)-2-methyl-propan-2-yl)-1H-indol-5-yl)cyclopropanecarboxamide

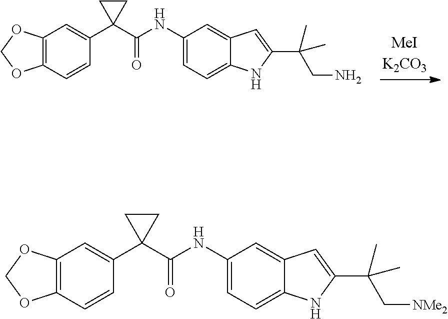 [SMILES: CC(C)(CN)c1cc2cc(NC(=O)C3(c4ccc5c(c4)OCO5)CC3)ccc2[nH]1 ; CN(C)CC(C)(C)c1cc2cc(NC(=O)C3(c4ccc5c(c4)OCO5)CC3)ccc2[nH]1]

To a solution of N-(2-(1-amino-2-methylpropan-2-yl)-1H-indol-5-yl)-1-(benzo[d][1,3]dioxol-5-yl)cyclopropanecarboxamide (20 mg, 0.051 mmol) in DMF (1 mL) was added potassium carbonate (35 mg, 0.26 mmol) and iodomethane (7.0 μL, 0.11 mmol). The mixture was stirred for 2 h. Water was added and the mixture was extracted with dichloromethane. Combined organic phases were dried over magnesium sulfate, evaporated, coevaporated with toluene (3×) and purified by silica gel chromatography (0-30% EtOAc in hexane) to give 1-(benzo[d][1,3]dioxol-5-yl)-N-(2-(1-(dimethylamino)-2-methylpropan-2-yl)-1H-indol-5-yl)cyclopropanecarboxamide (7 mg, 33%). ¹H NMR (400 MHz, CDCl₃) 9.74 (s, 1H), 7.58 (d, J=1.9 Hz, 1H), 7.20 (d, J=8.6 Hz, 1H), 7.15 (s, 1H), 7.01-6.95 (m, 3H), 6.85 (d, J=7.9 Hz, 1H), 6.10 (d, J=0.9 Hz, 1H), 6.02 (s, 2H), 2.43 (s, 2H), 2.24 (s, 6H), 1.68 (dd, J=3.7, 6.7 Hz, 2H), 1.33 (s, 6H), 1.08 (dd, J=3.7, 6.8 Hz, 2H).

### Example 115

### N-(2-(1-Acetamido-2-methylpropan-2-yl)-1H-indol-5-yl)-1-(benzo[d][1,3]-dioxol-5-yl)cyclopropanecarboxamide

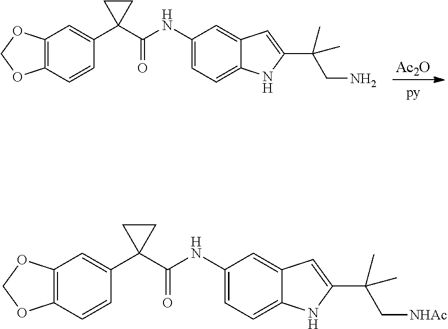 [SMILES: "Ac2O" ; CC(C)(CN)c1cc2cc(NC(=O)C3(c4ccc5c(c4)OCO5)CC3)ccc2[nH]1 ; CC(=O)NCC(C)(C)c1cc2cc(NC(=O)C3(c4ccc5c(c4)OCO5)CC3)ccc2[nH]1]

To a solution of N-(2-(1-amino-2-methylpropan-2-yl)-1H-indol-5-yl)-1-(benzo[d][1,3]dioxol-5-yl)cyclopropanecarboxamide (21 mg, 0.054 mmol) in dichloromethane (1 mL) was added pyridine (14 μL, 0.16 mmol) followed by acetic anhydride (6.0 μL, 0.059 mmol). The mixture was stirred for 2 h. Water was added and the mixture was extracted with dichloromethane, evaporated, coevaporated with toluene (3×) and purified by silica gel chromatography (60-100% ethylacetate in hexane) to give N-(2-(1-acetamido-2-methylpropan-2-yl)-1H-indol-5-yl)-1-(benzo[d][1,3]-dioxol-5-yl)cyclopropanecarboxamide (17 mg, 73%). ¹H NMR (400 MHz, DMSO) δ 10.79 (s, 1H), 8.39 (s, 1H), 7.66 (t, J=6.2 Hz, 1H), 7.56 (d, J=1.7 Hz, 1H), 7.18-7.14 (m, 1H), 7.02-6.89 (m, 4H), 6.08 (d, J=1.5 Hz, 1H), 6.03 (s, 2H), 3.31 (d, J=6.2 Hz, 2H), 1.80 (s, 3H), 1.41-1.38 (m, 2H), 1.26 (s, 6H), 1.04-1.01 (m, 2H).

### Example 116

### 1-(Benzo[d][1,3]dioxol-5-yl)-N-(2-(2-methyl-4-(1H-tetrazol-5-yl)butan-2-yl)-1H-indol-5-yl)cyclopropanecarboxamide

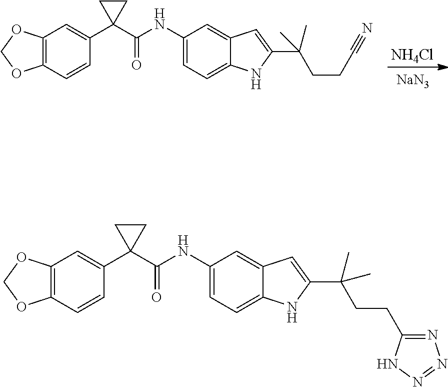 [SMILES: CC(C)(CCC#N)c1cc2cc(NC(=O)C3(c4ccc5c(c4)OCO5)CC3)ccc2[nH]1 ; "NH4Cl" ; CC(C)(CCc1nnn[nH]1)c1cc2cc(NC(=O)C3(c4ccc5c(c4)OCO5)CC3)ccc2[nH]1 ; "NaN3"]

1-(Benzo[d][1,3]dioxol-5-yl)-N-(2-(4-cyano-2-methylbutan-2-yl)-1H-indol-5-yl)cyclopropanecarboxamide (83 mg, 0.20 mmol) was dissolved in N,N-dimethylformamide (1 mL) containing ammonium chloride (128 mg, 2.41 mmol), sodium azide (156 mg, 2.40 mmol), and a magnetic stir bar. The reaction mixture was heated at 110° C. for 40 minutes in a microwave reactor. The crude product was filtered and then purified by preparative HPLC using a gradient of 0-99% acetonitrile in water containing 0.05% trifluoroacetic acid to yield 1-(benzo[d][1,3]dioxol-5-yl)-N-(2-(2-methyl-4-(1H-tetrazol-5-yl)butan-2-yl)-1H-indol-5-yl)cyclopropanecarboxamide. ESI-MS m/z calc. 458.2. found 459.2 (M+1)⁺. Retention time of 1.53 minutes. ¹H NMR (400 MHz, CD₃CN) 9.23 (s, 1H), 7.51-7.48 (m, 2H), 7.19 (d, J=8.6 Hz, 1H), 7.06-7.03 (m, 2H), 6.95-6.89 (m, 2H), 6.17 (dd, J=0.7, 2.2 Hz, 1H), 6.02 (s, 2H), 2.61-2.57 (m, 2H), 2.07-2.03 (m, 2H), 1.55-1.51 (m, 2H), 1.39 (s, 6H), 1.12-1.09 (m, 2H).

### Example 117

### 1-(Benzo[d][1,3]dioxol-5-yl)-N-(2-(piperidin-2-yl)-1H-indol-5-yl)cyclopropanecarboxamide

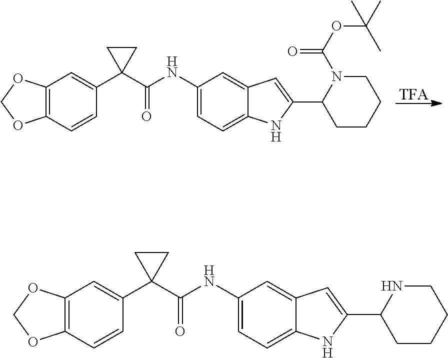 [SMILES: CC(C)(C)OC(=O)N1CCCCC1c1cc2cc(NC(=O)C3(c4ccc5c(c4)OCO5)CC3)ccc2[nH]1 ; O=C(Nc1ccc2[nH]c(C3CCCCN3)cc2c1)C1(c2ccc3c(c2)OCO3)CC1 ; "TFA"]

tert-Butyl 2-(5-(1-(benzo[d][1,3]dioxol-5-yl)cyclo-propanecarboxamido)-1H-indol-2-yl)piperidine-1-carboxylate (55 mg, 0.11 mmol) was dissolved in dichloromethane (2.5 mL) containing trifluoroacetic acid (1 mL). The reaction mixture was stirred for 6 h at room temperature. The crude product was purified by preparative HPLC using a gradient of 0-99% acetonitrile in water containing 0.05% trifluoroacetic acid to yield 1-(benzo[1,3]dioxol-5-yl)-N-(2-(piperidin-2-yl)-1H-indol-5-yl)cyclopropanecarboxamide. ESI-MS m/z calc. 403.2. found 404.4 (M+1)⁺. Retention time of 0.95 minutes.

### Example 118

### 5-tert-Butyl-1H-indol-6-ylamine

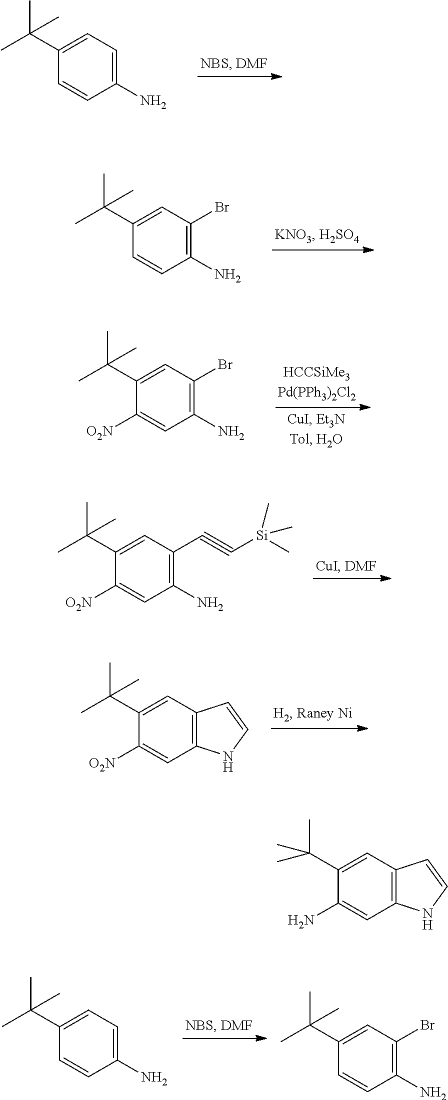

### 2-Bromo-4-tert-butyl-phenylamine

To a solution of 4-tert-Butyl-phenylamine (447 g, 3.00 mol) in DMF (500 mL) was added dropwise NBS (531 g, 3.00 mol) in DMF (500 mL) at room temperature. Upon completion, the reaction mixture was diluted with water and extracted with EtOAc. The organic layer was washed with water, brine, dried over Na₂SO₄ and concentrated. The crude product was directly used in the next step without further purification.

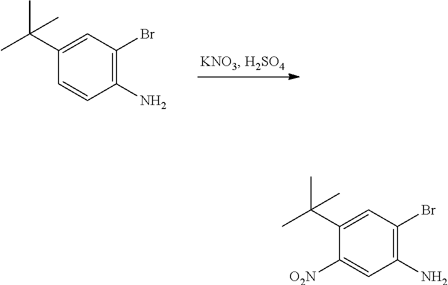

### 2-Bromo-4-tert-butyl-5-nitro-phenylamine

2-Bromo-4-tert-butyl-phenylamine (160 g, 0.71 mol) was added dropwise to H₂SO₄ (410 mL) at room temperature to yield a clear solution. This clear solution was then cooled down to −5 to −10° C. A solution of KNO₃ (83 g, 0.82 mol) in H₂SO₄ (410 mL) was added dropwise while the temperature was maintained between −5 to −10° C. Upon completion, the reaction mixture was poured into ice/water and extracted with EtOAc. The combined organic layers were washed with 5% Na₂CO₃ and brine, dried over Na₂SO₄ and concentrated. The residue was purified by a column chromatography (ethyl acetate/petroleum ether 1:10) to give 2-bromo-4-tert-butyl-5-nitro-phenylamine as a yellow solid (150 g, 78%).

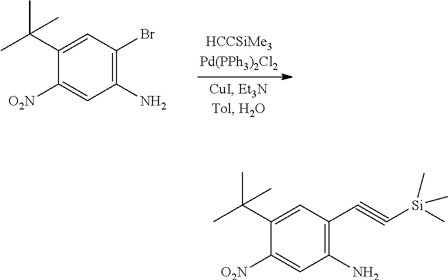 [SMILES: CC(C)(C)c1cc(Br)c(N)cc1[N+](=O)[O-] ; CC(C)(C)c1cc(C#C[Si](C)(C)C)c(N)cc1[N+](=O)[O-]]

### 4-tert-Butyl-5-nitro-2-trimethylsilanylethynyl-phenylamine

To a mixture of 2-bromo-4-tert-butyl-5-nitro-phenylamine (27.3 g, 100 mmol) in toluene (200 mL) and water (100 mL) was added Et₃N (27.9 mL, 200 mmol), Pd(PPh₃)2Cl₂ (2.11 g, 3.00 mmol), CuI (950 mg, 0.500 mmol) and trimethylsilyl acetylene (21.2 mL, 150 mmol) under a nitrogen atmosphere. The reaction mixture was heated at 70° C. in a sealed pressure flask for 2.5 h., cooled down to room temperature and filtered through a short plug of Celite. The filter cake was washed with EtOAc. The combined filtrate was washed with 5% NH₄OH solution and water, dried over Na₂SO₄ and concentrated. The crude product was purified by column chromatography (0-10% ethyl acetate/petroleum ether) to provide 4-tert-butyl-5-nitro-2-trimethylsilanylethynyl-phenylamine as a brown viscous liquid (25 g, 81%).

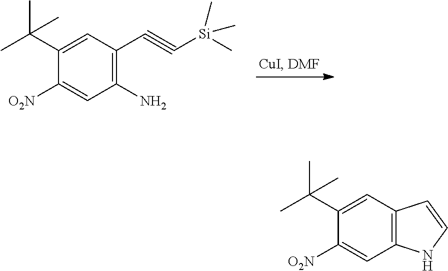 [SMILES: CC(C)(C)c1cc(C#C[Si](C)(C)C)c(N)cc1[N+](=O)[O-] ; CC(C)(C)c1cc2cc[nH]c2cc1[N+](=O)[O-]]

### 5-tert-Butyl-6-nitro-1H-indole

To a solution of 4-tert-butyl-5-nitro-2-trimethylsilanylethynyl-phenylamine (25 g, 86 mmol) in DMF (100 mL) was added CuI (8.2 g, 43 mmol) under a nitrogen atmosphere. The mixture was heated at 135° C. in a sealed pressure flask overnight, cooled down to room temperature and filtered through a short plug of Celite. The filter cake was washed with EtOAc. The combined filtrate was washed with water, dried over Na₂SO₄ and concentrated. The crude product was purified by column chromatography (10-20% ethyl acetate/hexane) to provide 5-tert-butyl-6-nitro-1H-indole as a yellow solid (13 g, 69%).

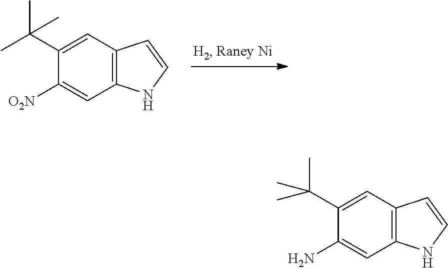 [SMILES: CC(C)(C)c1cc2cc[nH]c2cc1[N+](=O)[O-] ; CC(C)(C)c1cc2cc[nH]c2cc1N]

### 5-tert-Butyl-1H-indol-6-ylamine

Raney Nickel (3 g) was added to 5-tert-butyl-6-nitro-1H-indole (15 g, 67 mmol) in methanol (100 mL). The mixture was stirred under hydrogen (1 atm) at 30° C. for 3 h. The catalyst was filtered off. The filtrate was dried over Na₂SO₄ and concentrated. The crude dark brown viscous oil was purified by column chromatography (10-20% ethyl acetate/petroleum ether) to give 5-tert-butyl-1H-indol-6-ylamine as a gray solid (11 g, 87%). ¹H NMR (300 MHz, DMSO-d6) δ 10.3 (br s, 1H), 7.2 (s, 1H), 6.9 (m, 1H), 6.6 (s, 1H), 6.1 (m, 1H), 4.4 (br s, 2H), 1.3 (s, 9H).

A person skilled in the chemical arts can use the examples and schemes along with known synthetic methodologies to synthesize compounds of the present invention, including the compounds in Table II.D-3, below.

TABLE II
D-3: Physical data of exemplary compounds.

| Compound | LC/MS | LC/RT | |
|---|---|---|---|
| No. | M + 1 | Min | NMR |
| 1 | 373.3 | 2.49 | |
| 2 | 469.4 | 3.99 | |
| 3 | 381.3 | 3.69 | |
| 4 | 448.3 | 1.75 | |
| 5 | 389.3 | 3.3 | |
| 6 | 463 | 1.87 | |
| 7 | 363.3 | 3.7 | |
| 8 | 405.5 | 3.87 | |
| 9 | 487.3 | 2.12 | H NMR (400 |
| | | | MHz, |
| | | | DMSO-d6) δ |
| | | | 8.65 (s, 1H), |
| | | | 7.55 (d, J = |
| | | | 1.7 Hz, 1H), |
| | | | 7.49 (d, J = |
| | | | 1.4 Hz, 1H), |
| | | | 7.38 (d, J = |
| | | | 8.3 Hz, 1H), |
| | | | 7.30-7.25 (m, |
| | | | 2H), 7.08 |
| | | | (dd, J = 8.8, |
| | | | 1.9 Hz, 1H), |
| | | | 6.11 (s, 1H), |
| | | | 4.31 (t, J = |
| | | | 7.4 Hz, 2H), |
| | | | 3.64 (t, J = |
| | | | 7.3 Hz, 2H), |
| | | | 3.20 (t, J = |
| | | | 7.6 Hz, 2H), |
| | | | 1.92 (t, J = |
| | | | 7.6 Hz, 2H), |
| | | | 1.45 (m, 2H), |
| | | | 1.39 (s, 6H), |
| | | | 1.10 (m, 2H) |
| 10 | 388 | 334 | |
| 11 | 452.3 | 2.51 | |
| 12 | 527 | 2.36 | |
| 13 | 498 | 1.85 | |
| 14 | 404.5 | 1.18 | |
| 15 | 369.2 | 3.81 | |
| 16 | 419.2 | 2.24 | |
| 17 | 389.2 | 2.02 | H NMR (400 |
| | | | MHz, |
| | | | DMSO) δ |
| | | | 8.41 (s, 1H), |
| | | | 7.59 (d, J = |
| | | | 1.8 Hz, 1H), |
| | | | 7.15 (d, J = |
| | | | 8.6 Hz, 1H), |
| | | | 7.06-7.02 |
| | | | (m, 2H), |
| | | | 6.96-6.90 (m, |
| | | | 2H), 6.03 (s, |
| | | | 2H), 5.98 (d, |
| | | | J = 0.7 Hz, |
| | | | 1H), 4.06 (t, J = |
| | | | 6.8 Hz, |
| | | | 2H), 2.35 (t, |
| | | | J = 6.8 Hz, |
| | | | 2H), 1.42-1.38 |
| | | | (m, 2H), |
| | | | 1.34 (s, 6H), |
| | | | 1.05-1.01 (m, |
| | | | 2H) |
| 18 | 395.3 | 3.6 | H NMR (400 |
| | | | MHz, |
| | | | DMSO) δ |
| | | | 10.91 (s, 1H), |
| | | | 7.99 (s, 1H), |
| | | | 7.67 (d, J = |
| | | | 7.7 Hz, 1H), |
| | | | 7.08-6.92 (m, |
| | | | 4H), 6.09-6.03 |
| | | | (m, 3H), |
| | | | 1.47-1.42 |
| | | | (m, 2H), 1.31 |
| | | | (d, J = 7.3 |
| | | | Hz, 9H), |
| | | | 1.09-1.05 (m, |
| | | | 2H) |
| 19 | 457.2 | 1.97 | H NMR (400 |
| | | | MHz, |
| | | | CD3CN) |
| | | | 7.50 (d, J = |
| | | | 1.9 Hz, 1H), |
| | | | 7.41 (d, J = |
| | | | 1.6 Hz, 2H), |
| | | | 7.36 (dd, J = |
| | | | 1.7, 8.3 Hz, |
| | | | 1H), 7.29-7.24 |
| | | | (m, 2H), |
| | | | 7.02 (dd, J = |
| | | | 2.1, 8.8 Hz, |
| | | | 1H), 6.24 (s, |
| | | | 1H), 4.40 (t, |
| | | | J = 7.1 Hz, |
| | | | 2H), 3.80 (t, |
| | | | J = 7.1 Hz, |
| | | | 2H), 1.59-1.55 |
| | | | (m, 2H), |
| | | | 1.50 (s, 9H), |
| | | | 1.15-1.12 (m, |
| | | | 2H) |
| 20 | 375.5 | 3.71 | |
| 21 | 496 | 206 | |
| 22 | 421.14 | 1.53 | |
| 23 | 363.3 | 3.62 | |
| 24 | 378.5 | 2.66 | |
| 25 | 417.5 | 3.53 | |
| 26 | 454.3 | 3.18 | |
| 27 | 596.2 | 2.58 | |
| 28 | 379.3 | 2.92 | |
| 29 | 481 | 1.69 | |
| 30 | 504.2 | 1.95 | |
| 31 | 517 | 1.92 | |
| 32 | 403.5 | 3.5 | H NMR (400 |
| | | | MHz, |
| | | | DMSO) δ |
| | | | 10.76 (s, 1H), |
| | | | 8.72 (s, 1H), |
| | | | 7.79 (d, J = |
| | | | 2.3 Hz, 1H), |
| | | | 7.62 (dd, J = |
| | | | 2.4, 8.6 Hz, |
| | | | 1H), 7.55 (d, |
| | | | J = 1.5 Hz, |
| | | | 1H), 7.14 (d, |
| | | | J = 8.6 Hz, |
| | | | 1H), 7.05-7.01 |
| | | | (m, 2H), |
| | | | 6.03 (d, J = |
| | | | 1.6 Hz, 1H), |
| | | | 4.54 (t, J = |
| | | | 6.4 Hz, 2H), |
| | | | 2.79 (t, J = |
| | | | 6.4 Hz, 2H), |
| | | | 1.44 (m, 2H), |
| | | | 1.32 (s, 9H), |
| | | | 1.03 (m, 2H) |
| 33 | 321.3 | 2.98 | |
| 34 | 450.2 | 2.02 | |
| 35 | 395.1 | 3.59 | |
| 36 | 509 | 2.01 | |
| 37 | 447.2 | 2.02 | |
| 38 | 379.1 | 2.16 | H NMR (400 |
| | | | MHz, |
| | | | DMSO) δ |
| | | | 10.78 (s, 1H), |
| | | | 8.39 (s, 1H), |
| | | | 7.57 (d, J = |
| | | | 1.7 Hz, 1H), |
| | | | 7.17 (d, J = |
| | | | 8.6 Hz, 1H), |
| | | | 7.03-6.90 |
| | | | (m, 4H), 6.12 |
| | | | (d, J = 1.5 |
| | | | Hz, 1H), 6.03 |
| | | | (s, 2H), 5.18 |
| | | | (s, 1H), 1.50 |
| | | | (s, 6H), |
| | | | 1.41-1.38 (m, |
| | | | 2H), 1.05-0.97 |
| | | | (m, 2H) |
| 39 | 373.3 | 3.74 | |
| 40 | 372.8 | 3.8 | |
| 41 | 397.3 | 3.41 | H NMR (400 |
| | | | MHz, |
| | | | DMSO) δ |
| | | | 11.44 (s, 1H), |
| | | | 8.52 (s, 1H), |
| | | | 7.85 (d, J = |
| | | | 1.2 Hz, 2H), |
| | | | 7.71(d, J = |
| | | | 1.7 Hz, 1H), |
| | | | 7.47-7.43 |
| | | | (m, 2H), |
| | | | 7.32-7.26 (m, |
| | | | 2H), 7.12 |
| | | | (dd, J = 2.0, |
| | | | 8.7 Hz, 1H), |
| | | | 7.04 (d, J = |
| | | | 1.6 Hz, 1H), |
| | | | 6.97-6.90 |
| | | | (m, 2H), 6.84 |
| | | | (d, J = 1.3 |
| | | | Hz, 1H), 6.03 |
| | | | (s, 2H), |
| | | | 1.43-1.40 (m, |
| | | | 211), 1.07-1.03 |
| | | | (m, 2H) |
| 42 | 505.3 | 2.23 | H NMR (400 |
| | | | MHz, |
| | | | DMSO-d6) δ |
| | | | 8.33 (s, 1H), |
| | | | 7.52 (s, 1H), |
| | | | 7.42-7.39 (m, |
| | | | 2H), 7.33-7.25 |
| | | | (m, 2H), |
| | | | 6.14 (s, 1H), |
| | | | 4.99 (s, 1H), |
| | | | 4.31-4.27 (m, |
| | | | 3H), 3.64 (t, |
| | | | J = 7.0 Hz, |
| | | | 2H), 320 (t, |
| | | | J = 7.6 Hz, |
| | | | 2H), 1.91 (t, |
| | | | J = 7.6 Hz, |
| | | | 2H), 1.46 (m, |
| | | | 2H), 1.39 (s, |
| | | | 6H), 1.13 (m, |
| | | | 2H) |
| 43 | 505.4 | 1.97 | |
| 44 | 407.7 | 1.76 | H NMR (400 |
| | | | MHz, |
| | | | DMSO) δ |
| | | | 10.31 (s, 1H), |
| | | | 8.34 (s, 1H), |
| | | | 7.53 (d, J = |
| | | | 1.8 Hz, 1H), |
| | | | 7.03 (d, J = |
| | | | 1.6 Hz, 1H), |
| | | | 6.97-6.90 |
| | | | (m, 3H), |
| | | | 6.05-6.03 (m, |
| | | | 3H), 4.72 (s, |
| | | | 2H), 1.40-1.38 |
| | | | (m, 2H), |
| | | | 1.34 (s, 9H), |
| | | | 1.04-1.00(m, |
| | | | 2H) |
| 45 | 497.2 | 2.26 | |
| 46 | 391.3 | 3.41 | |
| 47 | 377.5 | 3.48 | |
| 48 | 427.5 | 4.09 | |
| 49 | 402.2 | 3.06 | |
| 50 | 421.1 | 1.81 | |
| 51 | 407.5 | 3.34 | |
| 52 | 464.3 | 2.87 | |
| 53 | 405.3 | 3.65 | |
| 54 | 375 | 1.84 | |
| 55 | 505.4 | 1.96 | |
| 56 | 335.3 | 3.18 | |
| 57 | 445.2 | 3.27 | |
| 58 | 491 | 1.88 | |
| 59 | 478 | 1.98 | |
| 60 | 413.3 | 3.95 | |
| 61 | 402.5 | 3.71 | |
| 62 | 393.3 | 1.98 | |
| 63 | 407.2 | 2.91 | |
| 64 | 505.4 | 1.98 | |
| 65 | 377.5 | 3.53 | |
| 66 | 417.5 | 4.06 | |
| 67 | 333.3 | 3.53 | |
| 68 | 397.3 | 3.86 | |
| 69 | 506 | 1.67 | |
| 70 | 501 | 2.1 | |
| 71 | 335.3 | 322 | |
| 72 | 487 | 1.93 | |
| 73 | 417.5 | 3.88 | |
| 74 | 395 | 1.95 | |
| 75 | 548 | 1.64 | |
| 76 | 418.3 | 2.9 | |
| 77 | 377.3 | 3.87 | |
| 78 | 363.3 | 3.48 | |
| 79 | 476 | 1.8 | |
| 80 | 447.3 | 2.18 | |
| 81 | 492.4 | 2 | |
| 82 | 564.3 | 1.35 | |
| 83 | 467.3 | 1.72 | |
| 84 | 445.2 | 3.08 | |
| 85 | 389.5 | 3.86 | |
| 86 | 374.3 | 3.11 | |
| 87 | 435 | 3.87 | |
| 88 | 465 | 1.89 | |
| 89 | 411.3 | 3.89 | |
| 90 | 449.3 | 3.92 | |
| 91 | 393.3 | 3.12 | |
| 92 | 469.6 | 1.75 | |
| 93 | 476.5 | 2.88 | |
| 94 | 377.5 | 3.41 | |
| 95 | 375.3 | 3.43 | H NMR (400 |
| | | | MHz, |
| | | | DMSO)δ |
| | | | 10.52 (s, 1H), |
| | | | 8.39 (s, 1H), |
| | | | 7.46 (d, J = |
| | | | 1.8 Hz, 1H), |
| | | | 7.10-6.89 |
| | | | (m, 5H), 6.03 |
| | | | (s, 2H), |
| | | | 2.68-2.65 (m, |
| | | | 2H), 2.56-2.54 |
| | | | (m, 2H), |
| | | | 1.82-1.77 |
| | | | (m, 4H), |
| | | | 1.41-1.34 (m, |
| | | | 2H), 1.04-0.97 |
| | | | (m, 2H) |
| 96 | 346.1 | 3.1 | |
| 97 | 367.3 | 3.72 | |
| 98 | 440.3 | 3.26 | |
| 99 | 393.1 | 3.18 | H NMR (400 |
| | | | MHz, |
| | | | DMSO-d6)δ |
| | | | 11.80 (s, 1H), |
| | | | 8.64 (s, 1H), |
| | | | 7.83 (m, 1H), |
| | | | 7.33-7.26 (m, |
| | | | 2H), 7.07 (m, |
| | | | 1H), 7.02 (m, |
| | | | 1H), 6.96-6.89 |
| | | | (m, 2H), |
| | | | 6.02 (s, 2H), |
| | | | 4.33 (q, J = |
| | | | 7.1 Hz, 2H), |
| | | | 1.42-1.39 (m, |
| | | | 2H), 1.33 (t, |
| | | | J = 7.1 Hz, |
| | | | 3H), 1.06-1.03 |
| | | | (m, 2H) |
| 100 | 421.3 | 1.85 | H NMR (400 |
| | | | MHz, |
| | | | DMSO) δ |
| | | | 13.05 (s, 1H), |
| | | | 9.96 (d, J = |
| | | | 1.6 Hz, 1H), |
| | | | 7.89 (d, J = |
| | | | 1.9 Hz, 1H), |
| | | | 7.74 (d, J = |
| | | | 2.0 Hz, 1H), |
| | | | 7.02 (d, J = |
| | | | 1.6 Hz, 1H), |
| | | | 6.96-6.88 (m, |
| | | | 2H), 6.22 (d, |
| | | | J = 2.3 Hz, |
| | | | 1H), 6.02 (s, |
| | | | 2H), 1.43-1.40 |
| | | | (m, 2H), |
| | | | 1.37 (s, 9H), |
| | | | 1.06-1.02 (m, |
| | | | 2H) |
| 101 | 387.5 | 2.51 | |
| 102 | 479 | 3.95 | |
| 103 | 420.3 | 3.12 | |
| 104 | 469.5 | 3.97 | |
| 105 | 391.3 | 2.04 | |
| 106 | 375.2 | 2.82 | |
| 107 | 349.3 | 3.33 | |
| 108 | 503.3 | 1.88 | |
| 109 | 451.5 | 1.59 | |
| 110 | 361.5 | 3.7 | |
| 111 | 391.3 | 3.65 | |
| 112 | 335.3 | 3.03 | |
| 113 | 496.5 | 1.68 | |
| 114 | 381.5 | 3.72 | |
| 115 | 390.3 | 3.22 | |
| 116 | 397.3 | 3.52 | H NMR (400 |
| | | | MHz, |
| | | | DMSO-d6) δ |
| | | | 11.27 (d, J = |
| | | | 1.9 Hz, 1H), |
| | | | 8.66 (s, 1H), |
| | | | 8.08 (d, J = |
| | | | 1.6 Hz, 1H), |
| | | | 7.65-7.61 (m, |
| | | | 3H), 7.46-7.40 |
| | | | (m, 2H), |
| | | | 7.31 (d, J = |
| | | | 8.7 Hz, 1H), |
| | | | 7.25-7.17 (m, |
| | | | 2H), 7.03 (d, |
| | | | J = 1.6 Hz, |
| | | | 1H), 6.98-6.87 |
| | | | (m, 2H), |
| | | | 6.02 (s, 2H), |
| | | | 1.43-1.39 (m, |
| | | | 2H), 1.06-1.02 |
| | | | (m, 2H) |
| 117 | 377.5 | 3.77 | |
| 118 | 515.3 | 2.3 | |
| 119 | 381.3 | 3.8 | |
| 120 | 464.2 | 2.1 | |
| 121 | 465 | 1.74 | |
| 122 | 395.2 | 3.74 | |
| 123 | 383.3 | 3.52 | |
| 124 | 388.5 | 3.56 | |
| 125 | 411.3 | 3.85 | |
| 126 | 459.2 | 1.53 | H NMR (400 |
| | | | MHz, |
| | | | CD3CN) δ |
| | | | 9.23 (s, 1H), |
| | | | 7.51-7.48 |
| | | | (m, 2H), 7.19 |
| | | | (d, J = 8.6 |
| | | | Hz, 1H), |
| | | | 7.06-7.03 (m, |
| | | | 2H), 6.95-6.89 |
| | | | (m, 2H), |
| | | | 6.17 (dd, J = |
| | | | 0.7, 2.2 Hz, |
| | | | 1H), 6.02 (s, |
| | | | 2H), 2.61-2.57 |
| | | | (m, 2H), |
| | | | 2.07-2.03 |
| | | | (m, 2H), |
| | | | 1.55-1.51 (m, |
| | | | 2H), 1.39 (s, |
| | | | 6H), 1.12-1.09 |
| | | | (m, 2H) |
| 127 | 408.5 | 2.48 | |
| 128 | 393 | 3.26 | |
| 129 | 420.2 | 2.16 | |
| 130 | 406.3 | 2.88 | |
| 131 | 473.3 | 4.22 | |
| 132 | 417.3 | 3.8 | |
| 133 | 465 | 1.74 | |
| 134 | 464.3 | 2.91 | |
| 135 | 347.3 | 3.42 | |
| 136 | 511 | 2.35 | |
| 137 | 455.5 | 3.29 | |
| 138 | 393.3 | 3.54 | |
| 139 | 335.1 | 3.08 | |
| 140 | 434.5 | 2.74 | |
| 141 | 381.3 | 2.91 | |
| 142 | 431.5 | 3.97 | |
| 143 | 539 | 1.89 | |
| 144 | 515 | 1.89 | |
| 145 | 407.5 | 3.6 | |
| 146 | 379.5 | 1.51 | |
| 147 | 409.3 | 4 | |
| 148 | 392.2 | 1.22 | |
| 149 | 375.3 | 3.37 | |
| 150 | 377.3 | 3.61 | |
| 151 | 377.22 | 3.96 | |
| 152 | 504.5 | 1.99 | |
| 153 | 393.1 | 3.47 | |
| 154 | 363.3 | 3.52 | |
| 155 | 321.3 | 3.13 | |
| 156 | 407.5 | 3.2 | |
| 157 | 406.3 | 1.43 | |
| 158 | 379.3 | 1.89 | |
| 159 | 451 | 3.34 | |
| 160 | 375.3 | 3.82 | |
| 161 | 355.1 | 3.32 | |
| 162 | 475 | 2.06 | |
| 163 | 437.2 | 2.35 | |
| 164 | 379.2 | 2.76 | |
| 165 | 462 | 3.44 | |
| 166 | 465.2 | 2.15 | |
| 167 | 455.2 | 2.45 | |
| 168 | 451 | 1.65 | |
| 169 | 528 | 1.71 | |
| 170 | 374.3 | 3.4 | |
| 171 | 449.5 | 1.95 | |
| 172 | 381.3 | 3.8 | |
| 173 | 346.3 | 2.93 | |
| 174 | 483.1 | 2.25 | |
| 175 | 411.2 | 3.85 | |
| 176 | 431.5 | 4.02 | |
| 177 | 485.5 | 4.02 | |
| 178 | 528.5 | 1.18 | |
| 179 | 473 | 1.79 | |
| 180 | 479 | 2.15 | |
| 181 | 387.5 | 2.56 | |
| 182 | 365.3 | 3.13 | |
| 183 | 493 | 2.3 | |
| 184 | 461.3 | 2.4 | H NMR (400 |
| | | | MHz, |
| | | | DMSO-d6)δ |
| | | | 10.89 (s, 1H), |
| | | | 8.29 (s, 1H), |
| | | | 7.52 (s, 1H), |
| | | | 7.42-7.37 (m, |
| | | | 2H), 7.32 |
| | | | (dd, J = 8.3, |
| | | | 1.4 Hz, 1H), |
| | | | 7.01 (d, J = |
| | | | 10.9 Hz, 1H), |
| | | | 6.05 (d, J = |
| | | | 1.7 Hz, 1H), |
| | | | 4.29 (t, J = |
| | | | 5.0 Hz, 1H), |
| | | | 3.23 (m, 2H), |
| | | | 1.81 (t, J = |
| | | | 7.7 Hz, 2H), |
| | | | 1.46 (m, 2H), |
| | | | 1.29 (s, 6H), |
| | | | 1.13 (m, 2H) |
| 185 | 377.5 | 3.63 | |
| 186 | 464 | 1.46 | |
| 187 | 339.1 | 3.2 | |
| 188 | 435.5 | 1.64 | |
| 189 | 392.3 | 2.18 | |
| 190 | 435.5 | 3.67 | H NMR (400 |
| | | | MHz, |
| | | | DMSO) δ |
| | | | 11.83 (s, 1H), |
| | | | 10.76 (s, 1H), |
| | | | 8.53 (s, 1H), |
| | | | 7.93 (d, J = |
| | | | 1.8 Hz, 1H), |
| | | | 7.60 (dd, J = |
| | | | 2.3, 8.5 Hz, |
| | | | 1H), 7.53 (d, |
| | | | J = 1.4 Hz, |
| | | | 1H), 7.14 (d, |
| | | | J = 8.6 Hz, |
| | | | 1H), 7.02-6.97 |
| | | | (m, 2H), |
| | | | 6.02 (d, J = |
| | | | 1.5 Hz, 1H), |
| | | | 3.71 (t, J = |
| | | | 6.2 Hz, 2H), |
| | | | 3.37 (t, J = |
| | | | 6.2 Hz, 2H), |
| | | | 3.25 (s, 3H), |
| | | | 1.44 (m, 2H), |
| | | | 1.32 (s, 9H), |
| | | | 1.08 (m, 2H) |
| 191 | 421.3 | 3.32 | |
| 192 | 404.4 | 0.95 | |
| 193 | 451 | 1.71 | |
| 194 | 465 | 1.69 | |
| 195 | 434.2 | 2.29 | |
| 196 | 363.3 | 3.4 | |
| 197 | 501 | 1.91 | |
| 198 | 411.2 | 3.14 | |
| 199 | 439 | 1.89 | |
| 200 | 434.4 | 1.53 | |
| 201 | 462 | 3.22 | |
| 202 | 351.3 | 2.59 | |
| 203 | 495.2 | 2.71 | |
| 204 | 435 | 3.94 | |
| 205 | 397.3 | 3.69 | |
| 206 | 493 | 2.26 | |
| 207 | 487 | 1.87 | |
| 208 | 391.3 | 2.94 | |
| 209 | 397.2 | 3.3 | |
| 210 | 487.2 | 1.85 | H NMR (400 |
| | | | MHz, |
| | | | CD3CN) δ |
| | | | 7.50 (d, J = |
| | | | 2.0 Hz, 1H), |
| | | | 7.41 (d, J = |
| | | | 1.6 Hz, 2H), |
| | | | 7.37-7.32 (m, |
| | | | 2H), 7.25 (d, |
| | | | J = 8.3 Hz, |
| | | | 1H), 6.98 |
| | | | (dd, J = 2.1, |
| | | | 8.8 Hz, 1H), |
| | | | 6.27 (d, J = |
| | | | 0.6 Hz, 1H), |
| | | | 4.40-4.28 |
| | | | (m, 2H), |
| | | | 4.12-4.06 (m, |
| | | | 1H), 3.59-3.51 |
| | | | (m, 2H), |
| | | | 1.59-1.50 |
| | | | (m, 2H), 1.47 |
| | | | (s, 9H), |
| | | | 1.15-1.12 (m, |
| | | | 2H) |
| 211 | 381.3 | 3.69 | |
| 212 | 461 | 2.04 | |
| 213 | 469 | 1.72 | |
| 214 | 363.3 | 3.48 | |
| 215 | 432.3 | 3.07 | |
| 216 | 403.5 | 3.94 | |
| 217 | 420.4 | 1.27 | |
| 218 | 475 | 2.2 | |
| 219 | 484.3 | 1.84 | |
| 220 | 419.3 | 3.87 | |
| 221 | 486.3 | 0.91 | |
| 222 | 391.3 | 3.01 | |
| 223 | 398.3 | 1.3 | |
| 224 | 349.2 | 2.54 | |
| 225 | 375.5 | 3.74 | |
| 226 | 377.5 | 3.47 | H NMR (400 |
| | | | MHz, |
| | | | DMSO-d6) δ |
| | | | 10.76 (s, 1H), |
| | | | 8.39 (s, 1H), |
| | | | 7.55 (s, 1H), |
| | | | 7.15-7.13 (m, |
| | | | 1H), 7.03-6.89 |
| | | | (m, 4H), |
| | | | 6.03 (m, 3H), |
| | | | 1.41-1.38 (m, |
| | | | 2H), 1.32 (s, |
| | | | 9H), 1.04-1.01 |
| | | | (m, 2H) |
| 227 | 393.3 | 2.03 | |
| 228 | 398.3 | 1.24 | |
| 229 | 487.2 | 1.78 | |
| 230 | 361.1 | 3.47 | |
| 231 | 435.5 | 2.12 | |
| 232 | 321.3 | 2.91 | |
| 233 | 413.3 | 3.77 | |
| 234 | 393.3 | 1.58 | |
| 235 | 465 | 1.92 | |
| 236 | 361.3 | 3.18 | |
| 237 | 421 | 1.8 | |
| 238 | 405.5 | 3.79 | |
| 239 | 544.3 | 1.4 | |
| 240 | 405.3 | 3.9 | |
| 241 | 462 | 1.74 | |
| 242 | 550 | 1.68 | |
| 243 | 395.2 | 1.98 | |
| 244 | 517.3 | 1.94 | |
| 245 | 372.2 | 3.59 | |
| 246 | 361.3 | 3.58 | |
| 247 | 490 | 1.95 | |
| 248 | 407.3 | 1.52 | H NMR (400 |
| | | | MHz, |
| | | | DMSO) δ |
| | | | 10.74 (d, J = |
| | | | 1.2 Hz, 1H), |
| | | | 8.40 (s, 1H), |
| | | | 7.54 (d, J = |
| | | | 1.8 Hz, 1H), |
| | | | 7.15 (d, J = |
| | | | 8.6 Hz, 1H), |
| | | | 7.03-6.90 |
| | | | (m, 4H), |
| | | | 6.03-6.00 (m, |
| | | | 3H), 3.26-3.22 |
| | | | (m, 2H), |
| | | | 1.85-1.80 (m, |
| | | | 2H), 1.41-1.38 |
| | | | (m, 2H), |
| | | | 1.31 (s, 6H), |
| | | | 1.05-1.01 (m, |
| | | | 2H) |
| 249 | 393.3 | 3.32 | |
| 250 | 406.2 | 2.08 | |
| 251 | 511 | 2.39 | |
| 252 | 379.3 | 3.3 | |
| 253 | 383 | 3.46 | |
| 254 | 401.2 | 3.26 | |
| 255 | 398.3 | 1.38 | |
| 256 | 512.5 | 1.96 | |
| 257 | 389.2 | 3.05 | |
| 258 | 321.3 | 3.02 | |
| 259 | 392.1 | 2.74 | |
| 260 | 462 | 1.81 | |
| 261 | 453 | 1.91 | |
| 262 | 349.3 | 3.22 | |
| 263 | 391.1 | 3.67 | H NMR (400 |
| | | | MHz, |
| | | | DMSO) |
| | | | 1.01-1.05 (dd, |
| | | | J = 4.0, 6.7 Hz, |
| | | | 2H), |
| | | | 1.41-1.39 (m, |
| | | | 11H), 3.81 (s, |
| | | | 3H), 6.03 (s, |
| | | | 2H), 6.15 (s, |
| | | | 1H), 6.96-6.90 |
| | | | (m, 2H), |
| | | | 7.02 (d, J = |
| | | | 1.6 Hz, 1H), |
| | | | 7.09 (dd, J = |
| | | | 2.0, 8.8 Hz, |
| | | | 1H), 7.25 (d, |
| | | | J = 8.8 Hz, |
| | | | 1H), 7.60 (d, |
| | | | J= 1.9 Hz, |
| | | | 1H), 8.46 (s, |
| | | | 1H) |
| 264 | 421.3 | 1.66 | H NMR (400 |
| | | | MHz, |
| | | | CD3CN) |
| | | | 8.78 (s, 1H), |
| | | | 7.40 (m, 1H), |
| | | | 7.33 (s, 1H), |
| | | | 7.08 (m, 1H), |
| | | | 6.95-6.87 |
| | | | (m, 3H), 6.79 |
| | | | (m, 1H), 5.91 |
| | | | (s, 2H), 3.51 |
| | | | (dd, J = 5.9, |
| | | | 7.8 Hz, 2H), |
| | | | 2.92-2.88 |
| | | | (m, 2H), 2.64 |
| | | | (t, J = 5.8 Hz, |
| | | | 1H), 1.50 (m, |
| | | | 2H), 1.41 (s, |
| | | | 9H), 1.06 (m, |
| | | | 2H) |
| 265 | 475 | 2.15 | |
| 266 | 347.3 | 3.32 | |
| 267 | 420.5 | 1.81 | |
| 268 | 416.2 | 1.76 | |
| 269 | 485 | 2.06 | |
| 270 | 395.3 | 3.89 | |
| 271 | 492 | 1.59 | |
| 272 | 405.5 | 3.96 | |
| 273 | 547.2 | 1.65 | |
| 274 | 631.6 | 1.91 | |
| 275 | 590.4 | 2.02 | |
| 276 | 465.7 | 1.79 | |
| 277 | 411.3 | 2.14 | |
| 278 | 385.3 | 1.99 | |
| 279 | 425.3 | 2.19 | |
| 280 | 473.2 | 1.74 | |
| 281 | 469.4 | 2.02 | H NMR (400 |
| | | | MHz, |
| | | | DMSO)δ |
| | | | 8.82 (s, 1H), |
| | | | 7.84 (d, J = |
| | | | 1.7 Hz, 1H), |
| | | | 7.55-7.51 |
| | | | (m, 2H), |
| | | | 7.40-7.35 (m, |
| | | | 2H), 7.29 |
| | | | (dd, J = 1.7, |
| | | | 8.3 Hz, 1H), |
| | | | 7.04 (s, 1H), |
| | | | 4.98 (t, J = |
| | | | 5.6 Hz, 1H), |
| | | | 4.27 (t, J = |
| | | | 6.1 Hz, 2H), |
| | | | 3.67 (q, J = |
| | | | 6.0 Hz, 2H), |
| | | | 1.48 (dd, J = |
| | | | 4.0, 6.7 Hz, |
| | | | 2H), 1.13 |
| | | | (dd, J = 4.1, |
| | | | 6.8 Hz, 2H) |
| 282 | 644.4 | 1.83 | |
| 283 | 544.6 | 1.97 | |
| 284 | 465.4 | 1.56 | |
| 285 | 485.2 | 1.8 | |
| 286 | 475.2 | 1.87 | |
| 287 | 564.2 | 1.95 | |
| 288 | 512.5 | 1.89 | H NMR (400 |
| | | | MHz, |
| | | | DMSO) δ |
| | | | 8.77 (s, 1H), |
| | | | 7.97 (s, 1H), |
| | | | 7.51 (s, 1H), |
| | | | 7.43-7.40 |
| | | | (m, 2H), 7.33 |
| | | | (d, J = 8.2 |
| | | | Hz, 1H), 6.36 |
| | | | (s, 1H), |
| | | | 4.99-4.97 (m, |
| | | | 2H), 4.52 (d, |
| | | | J = 13.1 Hz, |
| | | | 1H), 4.21 |
| | | | (dd, J = 9.2, |
| | | | 15.2 Hz, 1H), |
| | | | 3.86 (m, 1H), |
| | | | 3.51-3.36 |
| | | | (m, 2H), |
| | | | 1.51-1.48 (m, |
| | | | 2H), 1.43 (s, |
| | | | 9H), 1.17-1.15 |
| | | | (m, 2H) |
| 289 | 437.3 | 1.6 | |
| 290 | 499.5 | 1.81 | H NMR (400 |
| | | | MHz, |
| | | | DMSO) δ |
| | | | 8.82 (s, 1H), |
| | | | 7.83 (d, J = |
| | | | 1.7 Hz, 1H), |
| | | | 7.55-7.50 |
| | | | (m, 2H), |
| | | | 7.39-7.28 (m, |
| | | | 3H), 7.03 (s, |
| | | | 1H), 4.97 (d, |
| | | | J = 5.6 Hz, |
| | | | 1H), 4.83 (t, |
| | | | J = 5.6 Hz, |
| | | | 1H), 4.33 |
| | | | (dd, J = 3.4, |
| | | | 15.1 Hz, 1H), |
| | | | 4.09 (dd, J = |
| | | | 8.7, 15.1 Hz, |
| | | | 1H), 3.80-3.78 |
| | | | (m, 1H), |
| | | | 3.43-3.38 |
| | | | (m, 1H), |
| | | | 3.35-3.30 (m, |
| | | | 1H), 1.49-1.46 |
| | | | (m, 2H), |
| | | | 1.14-1.11 |
| | | | (m, 2H) |
| 291 | 455.4 | 2.02 | H NMR (400 |
| | | | MHz, |
| | | | DMSO) δ |
| | | | 8.62 (s, 1H), |
| | | | 7.56 (s, 1H), |
| | | | 7.50 (s, 1H), |
| | | | 7.38 (d, J = |
| | | | 8.3 Hz, 1H), |
| | | | 7.29 (dd, J = |
| | | | 1.5, 8.3 Hz, |
| | | | 1H), 7.23 (d, |
| | | | J = 8.7 Hz, |
| | | | 1H), 7.06 |
| | | | (dd, J = 1.7, |
| | | | 8.7 Hz, 1H), |
| | | | 6.19 (s, 1H), |
| | | | 4.86 (t, J = |
| | | | 5.4 Hz, 1H), |
| | | | 4.03 (t, J = |
| | | | 6.1 Hz, 2H), |
| | | | 3.73 (qn, J = |
| | | | 8.5 Hz, 1H), |
| | | | 3.57 (q, J = |
| | | | 5.9 Hz, 2H), |
| | | | 2.39-2.33 |
| | | | (m, 2H), |
| | | | 2.18-1.98 (m, |
| | | | 3H), 1.88-1.81 |
| | | | (m, 1H), |
| | | | 1.47-1.44 |
| | | | (m, 2H), |
| | | | 1.11-1.09 (m, |
| | | | 2H) |
| 292 | 578.4 | 1.99 | |
| 293 | 630.4 | 1.8 | |
| 294 | 443.4 | 1.98 | H NMR (400 |
| | | | MHz, |
| | | | DMSO) δ |
| | | | 8.62 (s, 1H), |
| | | | 7.55 (d, J = |
| | | | 1.8 Hz, 1H), |
| | | | 7.50 (d, J = |
| | | | 1.5 Hz, 1H), |
| | | | 7.38 (d, J = |
| | | | 8.3 Hz, 1H), |
| | | | 7.30-7.24 |
| | | | (m, 2H), 7.05 |
| | | | (dd, J = 2.0, |
| | | | 8.8 Hz, 1H), |
| | | | 6.13 (s, 1H), |
| | | | 4.88 (t, J = |
| | | | 5.5 Hz, 1H), |
| | | | 4.14 (t, J = |
| | | | 6.1 Hz, 2H), |
| | | | 3.61 (m, 2H), |
| | | | 3.21 (septet, |
| | | | J = 6.8 Hz, |
| | | | 1H), 1.47-1.44 |
| | | | (m, 2H), |
| | | | 1.26 (d, J = |
| | | | 6.8 Hz, 6H), |
| | | | 1.11-1.08 |
| | | | (m, 2H) |
| 295 | 482.3 | 2 | H NMR (400 |
| | | | MHz, |
| | | | DMSO) 8.78 |
| | | | (s, 1H), 7.92 |
| | | | (s, 1H), 7.51 |
| | | | (s, 1H), 7.45 |
| | | | (s, 1H), 7.41 |
| | | | (d, J = 8.3 |
| | | | Hz, 1H), 7.33 |
| | | | (d, J = 8.4 |
| | | | Hz, 1H), 6.34 |
| | | | (s, 1H), 5.01 |
| | | | (t, J = 5.7 Hz, |
| | | | 1H), 4.41 (t, |
| | | | J = 6.6 Hz, |
| | | | 2H), 3.68 (m, |
| | | | 2H), 1.51-1.47 |
| | | | (m, 2H), |
| | | | 1.42 (s, 9H), |
| | | | 1.19-1.15 |
| | | | (m, 2H) |
| 296 | 438.7 | 2.12 | H NMR (400 |
| | | | MHz, |
| | | | DMSO) δ |
| | | | 11.43 (s, 1H), |
| | | | 8.74 (s, 1H), |
| | | | 7.63 (s, 1H), |
| | | | 7.51 (s, 1H), |
| | | | 7.45-7.40 |
| | | | (m, 2H), 7.33 |
| | | | (dd, J = 1.4, |
| | | | 8.3 Hz, 1H), |
| | | | 6.25 (d, J = |
| | | | 1.5 Hz, 1H), |
| | | | 1.51-1.48 |
| | | | (m, 2H), 1.34 |
| | | | (s, 9H), |
| | | | 1.17-1.14 (m, |
| | | | 2H) |
| 297 | 449.3 | 1.6 | |
| 298 | 517.5 | 1.64 | |
| 299 | 391.5 | 2.05 | |
| 300 | 449.3 | 1.59 | |
| 301 | 501.2 | 1.93 | |
| 302 | 503.5 | 1.63 | |
| 303 | 437.3 | 1.6 | |
| 304 | 425.1 | 2.04 | H NMR (400 |
| | | | MHz, |
| | | | DMSO) δ |
| | | | 12.16 (s, 1H), |
| | | | 8.80 (s, 1H), |
| | | | 7.83 (s, 1H), |
| | | | 7.51 (d, J = |
| | | | 1.4 Hz, 1H), |
| | | | 7.39-7.28 |
| | | | (m, 4H), 6.95 |
| | | | (s, 1H), 1.48 |
| | | | (dd, J = 4.0, |
| | | | 6.6 Hz, 2H), |
| | | | 1.13 (dd, J = |
| | | | 4.0, 6.7 Hz, |
| | | | 2H) |
| 305 | 459.2 | 1.67 | |
| 306 | 558.4 | 2.05 | |
| 307 | 447.5 | 1.93 | |
| 308 | 516.7 | 1.69 | ^{J}H NMR |
| | | | (400 MHz, |
| | | | DMSO-d6) δ |
| | | | 8.32 (s, 1H), |
| | | | 7.53 (s, 1H), |
| | | | 7.43-7.31 |
| | | | (m, 4H), 6.19 |
| | | | (s, 1H), |
| | | | 4.95-4.93 (m, |
| | | | 2H), 4.51 (d, |
| | | | J = 5.0 Hz, |
| | | | 1H), 4.42-4.39 |
| | | | (m, 2H), |
| | | | 4.10-4.04 |
| | | | (m, 1H), 3.86 |
| | | | (s, 1H), |
| | | | 3.49-3.43 (m, |
| | | | 2H), 3.41-333 |
| | | | (m, 1H), |
| | | | 3.30-3.10 |
| | | | (m, 6H), |
| | | | 2.02-1.97 (m, |
| | | | 2H), 1.48-1.42 |
| | | | (m, 8H) |
| | | | and 1.13 (dd, |
| | | | J = 4.0, 6.7 |
| | | | Hz, 2H) ppm |
| 309 | 535.7 | 1.79 | 1H NMR |
| | | | (400.0 MHz, |
| | | | DMSO) δ |
| | | | 8.43 (s, 1H), |
| | | | 7.53 (s, 1H), |
| | | | 7.45-7.41 |
| | | | (m, 2H), |
| | | | 7.36-7.31 (m, |
| | | | 2H), 6.27 (s, |
| | | | 1H), 4.74-4.70 |
| | | | (m, 2H), |
| | | | 3.57-3.53 |
| | | | (m, 2H), 3.29 |
| | | | (s, 9H), |
| | | | 1.48-1.42 (m, |
| | | | 11H), and |
| | | | 1.15 (dd, J = |
| | | | 3.9, 6.8 Hz, |
| | | | 2H) ppm. |
| 310 | 609.5 | 1.64 | |
| 311 | 535.7 | 1.7 | ^{l}H NMR |
| | | | (400 MHz, |
| | | | DMSO-d6) δ |
| | | | 8.32 (s, 1H), |
| | | | 7.53 (d, J = |
| | | | 1.0 Hz, 1H), |
| | | | 7.43-7.31 |
| | | | (m, 4H), 6.17 |
| | | | (s, 1H), |
| | | | 4.97-4.92 (m, |
| | | | 2H),4.41 |
| | | | (dd, J = 2.4, |
| | | | 15.0 Hz, 1H), |
| | | | 4.23 (t, J = |
| | | | 5.0 Hz, 1H), |
| | | | 4.08 (dd, J = |
| | | | 8.6, 15.1 Hz, |
| | | | 1H), 3.87 (s, |
| | | | 1H), 3.48-3.44 |
| | | | (m, 1H), |
| | | | 3.41-3.33 |
| | | | (m, 1H), 3.20 |
| | | | (dd, J = 7.4, |
| | | | 12.7 Hz, 2H), |
| | | | 1.94-1.90 |
| | | | (m, 2H), |
| | | | 1.48-1.45 (m, |
| | | | 2H), 1.42 (s, |
| | | | 3H), 1.41 (s, |
| | | | 3H) and |
| | | | 1.15-1.12 (m, |
| | | | 2H) ppm. |
| 312 | 443 | 2.31 | ^{l}H NMR |
| | | | (400 MHz, |
| | | | DMSO-d6) δ |
| | | | 8.93 (s, 1H), |
| | | | 7.71 (d, J = |
| | | | 8.8 Hz, 1H), |
| | | | 7.51 (s, 1H), |
| | | | 7.42 (d, J = |
| | | | 8.3 Hz, 1H), |
| | | | 7.33 (d, J = |
| | | | 1.6 Hz, 1H), |
| | | | 7.08 (d, J = |
| | | | 8.8 Hz, 1H), |
| | | | 6.28 (s, 1H), |
| | | | 5.05 (t, J = |
| | | | 5.6 Hz, 1H), |
| | | | 4.42 (t, J = |
| | | | 6.8 Hz, 2H), |
| | | | 3.70-3.65 |
| | | | (m, 2H), |
| | | | 1.51-1.48 (m, |
| | | | 2H), 1.44 (s, |
| | | | 9H), 1.19-1.16 |
| | | | (m, 2H) |
| | | | ppm. |
| 313 | 521.5 | 1.69 | 1H NMR |
| | | | (400.0 MHz, |
| | | | CD3CN) d |
| | | | 7.69 (d, J = |
| | | | 7.7 Hz, 1H), |
| | | | 7.44 (d, J = |
| | | | 1.6 Hz, 1H), |
| | | | 7.39 (dd, J = |
| | | | 1.7, 8.3 Hz, |
| | | | 1H), 7.31 (s, |
| | | | 1H), 7.27 (d, |
| | | | J = 8.3 Hz, |
| | | | 1H), 7.20 (d, |
| | | | J = 12.0 Hz |
| | | | 1H), 6.34 (s, |
| | | | 1H), 4.32 (d, |
| | | | J = 6.8 Hz, |
| | | | 2H), 4.15-4.09 |
| | | | (m, 1H), |
| | | | 3.89 (dd, J = |
| | | | 6.0, 11.5 Hz, |
| | | | 1H), 3.63-3.52 |
| | | | (m, 3H), |
| | | | 3.42 (d, J = |
| | | | 4.6 Hz, 1H), |
| | | | 3.21 (dd, J = |
| | | | 6.2, 7.2 Hz, |
| | | | 1H), 3.04 (t, |
| | | | J = 5.8 Hz, |
| | | | 1H), 1.59 |
| | | | (dd, J = 3.8, |
| | | | 6.8 Hz, 2H), |
| | | | 1.44 (s, 3H), |
| | | | 1.33 (s, 3H) |
| | | | and 1.18 (dd, |
| | | | J = 3.7, 6.8 |
| | | | Hz, 2H) ppm |
| 314 | 447.5 | 1.86 | ^{l}H NMR |
| | | | (400 MHz, |
| | | | DMSO-d6) δ |
| | | | 8.20 (d, J = |
| | | | 7.6 Hz, 1H), |
| | | | 7.30-7.25 |
| | | | (m, 3H), 7.20 |
| | | | (in, 1H), 7.12 |
| | | | (d, J = 8.2 |
| | | | Hz, 1H), 6.84 |
| | | | (d, J = 11.1 |
| | | | Hz, 1H), 6.01 |
| | | | (d, J = 0.5 |
| | | | Hz, 1H), 3.98 |
| | | | (t, J = 6.8 Hz, |
| | | | 2H), 2.37 (t, |
| | | | J = 6.8 Hz, |
| | | | 2H), 1.75 |
| | | | (dd, J = 3.8, |
| | | | 6.9 Hz, 2H), |
| | | | 1.37 (s, 6H) |
| | | | and 1.14 (dd, |
| | | | J = 3.9, 6.9 |
| | | | Hz, 2H) ppm. |
| 315 | 482.5 | 1.99 | H NMR (400 |
| | | | MHz, |
| | | | DMSO) 8.93 |
| | | | (s, 1H), 7.71 |
| | | | (d, J = 8.8 |
| | | | Hz, 1H),7.51 |
| | | | (s, 1H), 7.42 |
| | | | (d, J = 8.3 |
| | | | Hz, 1H), 7.33 |
| | | | (d, J = 1.6 |
| | | | Hz, 1H), 7.08 |
| | | | (d, J = 8.8 |
| | | | Hz, 1H), 6.28 |
| | | | (s, 1H), 5.05 |
| | | | (t, J = 5.6 Hz, |
| | | | 1H), 4.42 (t, |
| | | | J = 6.8 Hz, |
| | | | 2H), 3.70-3.65 |
| | | | (m, 2H), |
| | | | 1.51-1.48 |
| | | | (m, 2H), 1.44 |
| | | | (s, 9H), |
| | | | 1.19-1.16 (m, |
| | | | 2H) |
| 316 | 438.7 | 2.1 | H NMR (400 |
| | | | MHz, |
| | | | DMSO) |
| | | | 11.48 (s, 1H), |
| | | | 8.88 (s, 1H), |
| | | | 7.52 (d, J = |
| | | | 8.5 Hz, 2H), |
| | | | 7.41 (d, J = |
| | | | 8.3 Hz, 1H), |
| | | | 7.32 (dd, J = |
| | | | 1.5, 8.3 Hz, |
| | | | 1H), 7.03 (d, |
| | | | J = 8.6 Hz, |
| | | | 1H), 621 (d, |
| | | | J = 1.8 Hz, |
| | | | 1H), 1.51-1.49 |
| | | | (m, 2H), |
| | | | 1.36 (s, 9H), |
| | | | 1.18-1.16 |
| | | | (m, 2H) ppm. |
| 317 | 439.4 | 1.36 | |
| 318 | 469.016 | 1.66 | |
| 319 | 469.016 | 1.66 | |
| 320 | 465.7 | 1.79 | H NMR (400 |
| | | | MHz, |
| | | | DMSO) 9.26 |
| | | | (s, 1H), 7.65 |
| | | | (d, J = 1.9 |
| | | | Hz, 1H), 7.49 |
| | | | (d, J = 8.7 |
| | | | Hz, 2H), 7.36 |
| | | | (d, J = 8.9 |
| | | | Hz, 1H), 7.11 |
| | | | (dd, J = 1.9, |
| | | | 8.9 Hz, 1H), |
| | | | 6.89 (d, J = |
| | | | 8.8 Hz, 2H), |
| | | | 6.14 (s, 1H), |
| | | | 4.42-4.37 |
| | | | (m, 1H), |
| | | | 4.16-4.10 (m, |
| | | | 1H), 3.90-3.88 |
| | | | (m, 1H), |
| | | | 3.73 (s, 3H), |
| | | | 3.46-3.42 |
| | | | (m, 2H), 1.41 |
| | | | (s, 9H), 1.36 |
| | | | (d, J = 5.0 |
| | | | Hz, 1H), 1.21 |
| | | | (s, 3H), 0.99 |
| | | | (d, J = 5.0 |
| | | | Hz, 1H), 0.84 |
| | | | (s, 3H) |
| 321 | 391.5 | 2.05 | H NMR (400 |
| | | | MHz, |
| | | | DMSO) |
| | | | 10.73 (s, 1H), |
| | | | 9.23 (s, 1H), |
| | | | 7.61 (d, J = |
| | | | 1.5 Hz, 1H), |
| | | | 7.49 (d, J = |
| | | | 8.8 Hz, 2H), |
| | | | 7.13 (s, 1H), |
| | | | 7.10 (d, J = |
| | | | 1.9 Hz, 1H), |
| | | | 6.88 (d, J = |
| | | | 8.8 Hz, 2H), |
| | | | 6.02 (d, J = |
| | | | 1.8 Hz, 1H), |
| | | | 3.73 (s, 3H), |
| | | | 1.36 (d, J = |
| | | | 5.0 Hz, 1H), |
| | | | 1.31 (s, 9H), |
| | | | 1.22 (s, 3H), |
| | | | 0.98 (d, J = |
| | | | 5.0 Hz, 1H), |
| | | | 0.84 (s, 3H) |
| 322 | 521.5 | 1.67 | 1H NMR |
| | | | (400.0 MHz, |
| | | | DMSO) d |
| | | | 8.31 (s, 1H), |
| | | | 7.53 (d, J = |
| | | | 1.1 Hz, 1H), |
| | | | 7.42-7.37 |
| | | | (m, 2H), |
| | | | 7.33-7.30 (m, |
| | | | 2H), 6.22 (s, |
| | | | 1H), 5.01 (d, |
| | | | J = 5.0 Hz, |
| | | | 1H), 4.91 (t, |
| | | | J = 5.5 Hz, |
| | | | 1H), 4.75 (t, |
| | | | J = 5.8 Hz, |
| | | | 1H), 4.42-4.38 |
| | | | (m, 1H), |
| | | | 4.10 (dd, J = |
| | | | 8.8, 15.1 Hz, |
| | | | 1H), 3.90 (s, |
| | | | 1H), 3.64-3.54 |
| | | | (m, 2H), |
| | | | 3.48-3.33 |
| | | | (m, 2H), |
| | | | 1.48-1.45 (m, |
| | | | 2H), 1.35 (s, |
| | | | 3H), 1.32 (s, |
| | | | 3H) and |
| | | | 1.14-1.11 (m, |
| | | | 2H) ppm |

### II.D.2. Compound of Formula D1

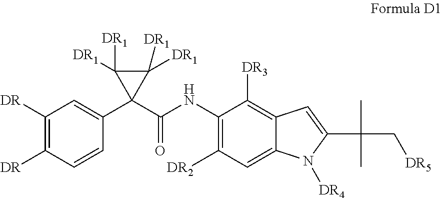

or pharmaceutically acceptable salts thereof, wherein:
DR is H, OH, OCH₃ or two R taken together form —OCH₂O— or —OCF₂O—;
DR₄ is H or alkyl;

### DR₅ is H or F;

### DR₆ is H or CN;

DR₇ is H, —CH₂CH(OH)CH₂OH, —CH₂CH₂N⁺(CH₃)₃, or —CH₂CH₂OH; DR₈ is H, OH, —CH₂CH(OH)CH₂OH, —CH₂OH, or DR₇ and DR₈ taken together form a five membered ring.

### II.D.3 Compound 3

In another embodiment, the compound of Formula D1 is Compound 3, which is known by its chemical name (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide.

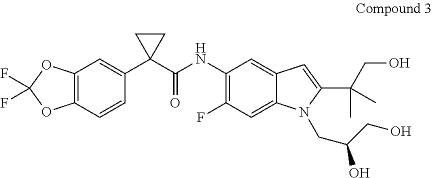 [SMILES: CC(C)(CO)c1cc2cc(NC(=O)C3(c4ccc5c(c4)OC(F)(F)O5)CC3)c(F)cc2n1C[C@H](O)CO]

### 1. Synthesis of Compounds of Formula D1

### a. General Schemes

Compound 3 can be prepared by coupling an acid chloride moiety with an amine moiety according to Schemes 3-1 through 3-3.

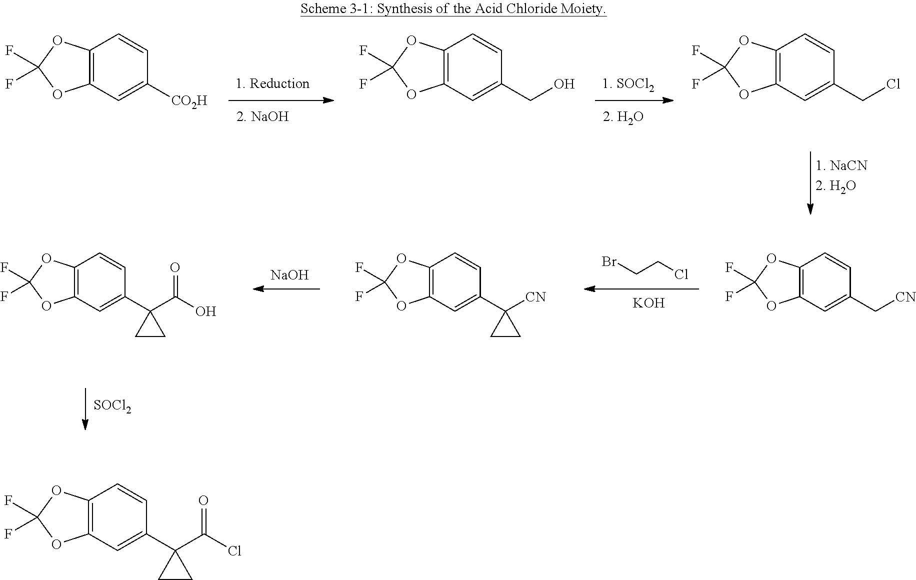

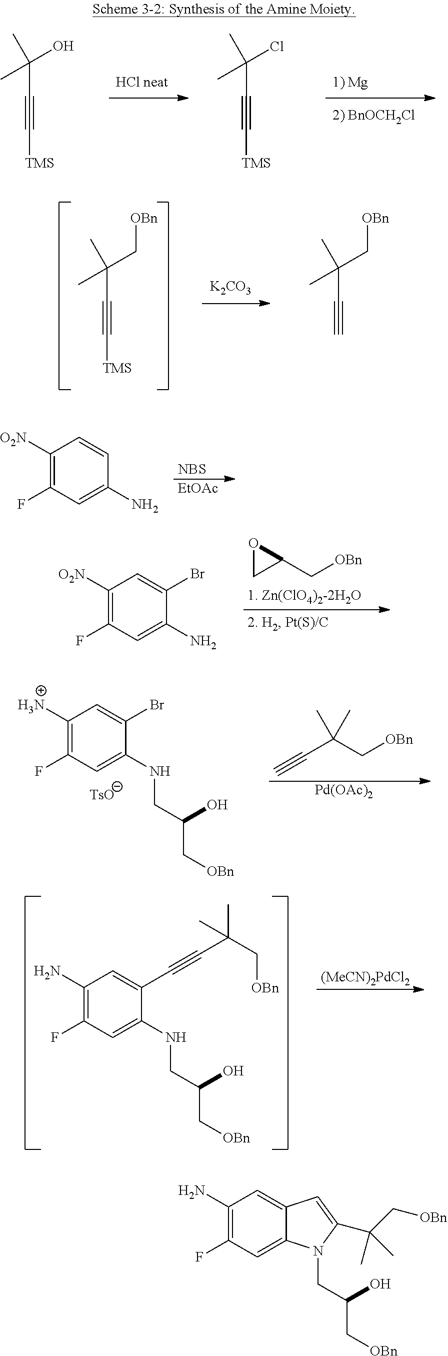

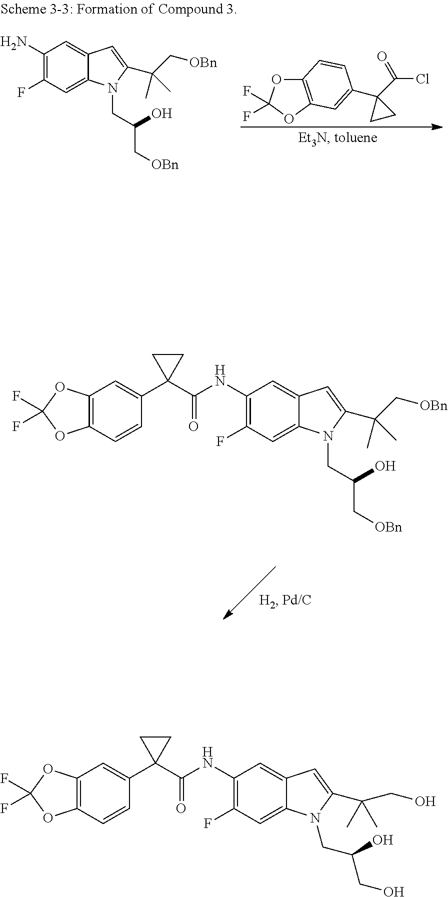 [SMILES: CC(C)(COCc1ccccc1)c1cc2cc(N)c(F)cc2n1C[C@@H](O)COCc1ccccc1 ; O=C(Cl)C1(c2ccc3c(c2)OC(F)(F)O3)CC1 ; CC(C)(COCc1ccccc1)c1cc2cc(NC(=O)C3(c4ccc5c(c4)OC(F)(F)O5)CC3)c(F)cc2n1C[C@@H](O)COCc1ccccc1 ; CC(C)(CO)c1cc2cc(NC(=O)C3(c4ccc5c(c4)OC(F)(F)O5)CC3)c(F)cc2n1C[C@@H](O)CO]

### b. Examples

Vitride® (sodium bis(2-methoxyethoxy)aluminum hydride [or NaAlH2(OCH2CH2OCH3)2], 65 wgt % solution in toluene) was purchased from Aldrich Chemicals.

2,2-Difluoro-1,3-benzodioxole-5-carboxylic acid was purchased from Saltigo (an affiliate of the Lanxess Corporation).

### Compound 3 Acid Moiety Synthesis

### (2,2-Difluoro-1,3-benzodioxol-5-yl)-methanol

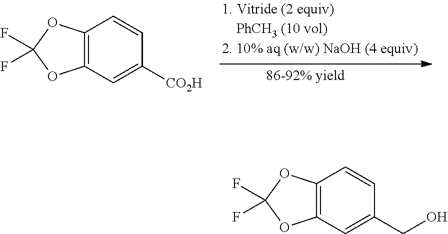 [SMILES: O=C(O)c1ccc2c(c1)OC(F)(F)O2 ; OCc1ccc2c(c1)OC(F)(F)O2]

Commercially available 2,2-difluoro-1,3-benzodioxole-5-carboxylic acid (1.0 eq) was slurried in toluene (10 vol). Vitride® (2 eq) was added via addition funnel at a rate to maintain the temperature at 15-25° C. At the end of addition the temperature was increased to 40° C. for 2 hours (h) then 10% (w/w) aq. NaOH (4.0 eq) was carefully added via addition funnel maintaining the temperature at 40-50° C. After stirring for an additional 30 minutes (min), the layers were allowed to separate at 40° C. The organic phase was cooled to 20° C. then washed with water (2×1.5 vol), dried (Na2SO4), filtered, and concentrated to afford crude (2,2-difluoro-1,3-benzodioxol-5-yl)-methanol that was used directly in the next step.

### 5-Chloromethyl-2,2-difluoro-1,3-benzodioxole

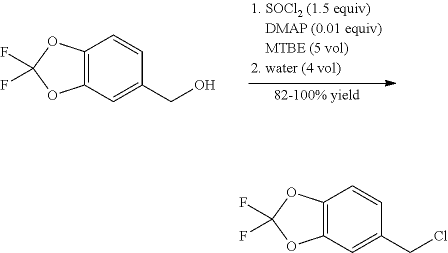 [SMILES: OCc1ccc2c(c1)OC(F)(F)O2 ; FC1(F)Oc2ccc(CCl)cc2O1]

(2,2-difluoro-1,3-benzodioxol-5-yl)-methanol (1.0 eq) was dissolved in MTBE (5 vol). A catalytic amount of DMAP (1 mol %) was added and SOCl2 (1.2 eq) was added via addition funnel. The SOCl2 was added at a rate to maintain the temperature in the reactor at 15-25° C. The temperature was increased to 30° C. for 1 hour then cooled to 20° C. then water (4 vol) was added via addition funnel maintaining the temperature at less than 30° C. After stirring for an additional 30 minutes, the layers were allowed to separate. The organic layer was stirred and 10% (w/v) aq. NaOH (4.4 vol) was added. After stirring for 15 to 20 minutes, the layers were allowed to separate. The organic phase was then dried (Na2SO4), filtered, and concentrated to afford crude 5-chloromethyl-2,2-difluoro-1,3-benzodioxole that was used directly in the next step.

### (2,2-Difluoro-1,3-benzodioxol-5-yl)-acetonitrile

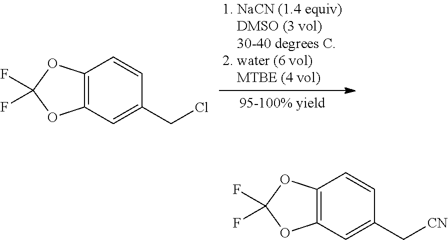 [SMILES: FC1(F)Oc2ccc(CCl)cc2O1 ; N#CCc1ccc2c(c1)OC(F)(F)O2]

A solution of 5-chloromethyl-2,2-difluoro-1,3-benzodioxole (1 eq) in DMSO (1.25 vol) was added to a slurry of NaCN (1.4 eq) in DMSO (3 vol) maintaining the temperature between 30-40° C. The mixture was stirred for 1 hour then water (6 vol) was added followed by MTBE (4 vol). After stirring for 30 min, the layers were separated. The aqueous layer was extracted with MTBE (1.8 vol). The combined organic layers were washed with water (1.8 vol), dried (Na2SO4), filtered, and concentrated to afford crude (2,2-difluoro-1,3-benzodioxol-5-yl)-acetonitrile (95%) that was used directly in the next step. 1H NMR (500 MHz, DMSO) δ 7.44 (br s, 1H), 7.43 (d, J=8.4 Hz, 1H), 7.22 (dd, J=8.2, 1.8 Hz, 1H), 4.07 (s, 2H).

### (2,2-Difluoro-1,3-benzodioxol-5-yl)-cyclopropanecarbonitrile

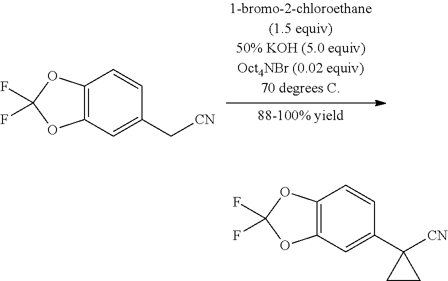 [SMILES: N#CCc1ccc2c(c1)OC(F)(F)O2 ; N#CC1(c2ccc3c(c2)OC(F)(F)O3)CC1]

A mixture of (2,2-difluoro-1,3-benzodioxol-5-yl)-acetonitrile (1.0 eq), 50 wt % aqueous KOH (5.0 eq) 1-bromo-2-chloroethane (1.5 eq), and Oct4NBr (0.02 eq) was heated at 70° C. for 1 h. The reaction mixture was cooled then worked up with MTBE and water. The organic phase was washed with water and brine then the solvent was removed to afford (2,2-difluoro-1,3-benzodioxol-5-yl)-cyclopropanecarbonitrile. 1H NMR (500 MHz, DMSO) δ 7.43 (d, J=8.4 Hz, 1H), 7.40 (d, J=1.9 Hz, 1H), 7.30 (dd, J=8.4, 1.9 Hz, 1H), 1.75 (m, 2H), 1.53 (m, 2H).

### 1-(2,2-Difluoro-1,3-benzodioxol-5-yl)-cyclopropanecarboxylic Acid

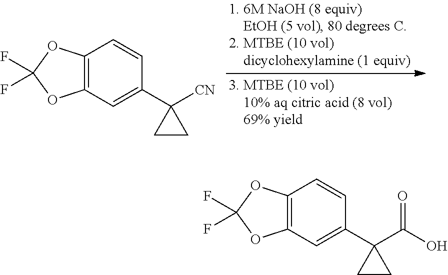 [SMILES: N#CC1(c2ccc3c(c2)OC(F)(F)O3)CC1 ; O=C(O)C1(c2ccc3c(c2)OC(F)(F)O3)CC1]

(2,2-difluoro-1,3-benzodioxol-5-yl)-cyclopropanecarbonitrile was hydrolyzed using 6 M NaOH (8 equiv) in ethanol (5 vol) at 80° C. overnight. The mixture was cooled to room temperature and ethanol was evaporated under vacuum. The residue was taken into water and MTBE, 1 M HCl was added and the layers were separated. The MTBE layer was then treated with dicyclohexylamine (0.97 equiv). The slurry was cooled to 0° C., filtered and washed with heptane to give the corresponding DCHA salt. The salt was taken into MTBE and 10% citric acid and stirred until all solids dissolve. The layers were separated and the MTBE layer was washed with water and brine. Solvent swap to heptane followed by filtration gives 1-(2,2-difluoro-1,3-benzodioxol-5-yl)-cyclopropanecarboxylic acid after drying in a vacuum oven at 50° C. overnight. ESI-MS m/z calc. 242.04. found 241.58 (M+1)+; 1H NMR (500 MHz, DMSO) δ 12.40 (s, 1H), 7.40 (d, J=1.6 Hz, 1H), 7.30 (d, J=8.3 Hz, 1H), 7.17 (dd, J=8.3, 1.7 Hz, 1H), 1.46 (m, 2H), 1.17 (m, 2H).

### Compound 3 Amine Moiety Synthesis

### 2-Bromo-5-fluoro-4-nitroaniline

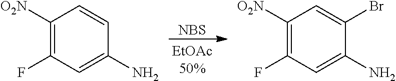

A flask was charged with 3-fluoro-4-nitroaniline (1.0 equiv) followed by ethyl acetate (10 vol) and stirred to dissolve all solids. N-Bromosuccinimide (1.0 equiv) was added portion-wise as to maintain an internal temperature of 22° C. At the end of the reaction, the reaction mixture was concentrated in vacuo on a rotavap. The residue was slurried in distilled water (5 vol) to dissolve and remove succinimide. (The succinimide can also be removed by water workup procedure.) The water was decanted and the solid was slurried in 2-propanol (5 vol) overnight. The resulting slurry was filtered and the wetcake was washed with 2-propanol, dried in vacuum oven at 50° C. overnight with N2 bleed until constant weight was achieved. A yellowish tan solid was isolated (50% yield, 97.5% AUC). Other impurities were a bromo-regioisomer (1.4% AUC) and a di-bromo adduct (1.1% AUC). 1H NMR (500 MHz, DMSO) δ 8.19 (1H, d, J=8.1 Hz), 7.06 (br. s, 2H), 6.64 (d, 1H, J=14.3 Hz).

### Benzylglycolated-4-ammonium-2-bromo-5-fluoroaniline tosylate salt

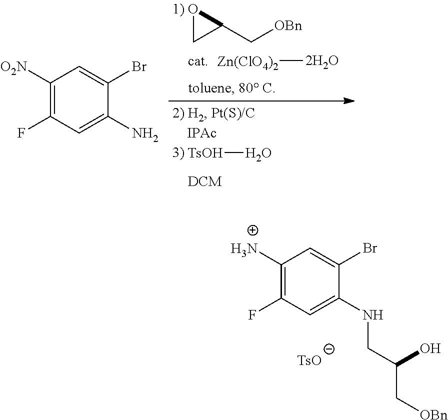

A thoroughly dried flask under N2 was charged with the following: Activated powdered 4 Å molecular sieves (50 wt % based on 2-bromo-5-fluoro-4-nitroaniline), 2-Bromo-5-fluoro-4-nitroaniline (1.0 equiv), zinc perchlorate dihydrate (20 mol %), and toluene (8 vol). The mixture was stirred at room temperature for no more than 30 min. Lastly, (R)-benzyl glycidyl ether (2.0 equiv) in toluene (2 vol) was added in a steady stream. The reaction was heated to 80° C. (internal temperature) and stirred for approximately 7 hours or until 2-Bromo-5-fluoro-4-nitroaniline was <5% AUC.

The reaction was cooled to room temperature and Celite® (50 wt %) was added, followed by ethyl acetate (10 vol). The resulting mixture was filtered to remove Celite® and sieves and washed with ethyl acetate (2 vol). The filtrate was washed with ammonium chloride solution (4 vol, 20% w/v). The organic layer was washed with sodium bicarbonate solution (4 vol×2.5% w/v). The organic layer was concentrated in vacuo on a rotovap. The resulting slurry was dissolved in isopropyl acetate (10 vol) and this solution was transferred to a Buchi hydrogenator.

The hydrogenator was charged with 5 wt % Pt(S)/C (1.5 mol %) and the mixture was stirred under N2 at 30° C. (internal temperature). The reaction was flushed with N2 followed by hydrogen. The hydrogenator pressure was adjusted to 1 Bar of hydrogen and the mixture was stirred rapidly (>1200 rpm). At the end of the reaction, the catalyst was filtered through a pad of Celite® and washed with dichloromethane (10 vol). The filtrate was concentrated in vacuo. Any remaining isopropyl acetate was chased with dichloromethane (2 vol) and concentrated on a rotavap to dryness.

The resulting residue was dissolved in dichloromethane (10 vol). p-Toluenesulfonic acid monohydrate (1.2 equiv) was added and stirred overnight. The product was filtered and washed with dichloromethane (2 vol) and suction dried. The wetcake was transferred to drying trays and into a vacuum oven and dried at 45° C. with N2 bleed until constant weight was achieved. Benzylglycolated-4-ammonium-2-bromo-5-fluoroaniline tosylate salt was isolated as an off-white solid.

### (3-Chloro-3-methylbut-1-ynyl)trimethylsilane

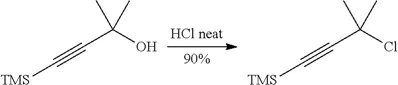

Propargyl alcohol (1.0 equiv) was charged to a vessel. Aqueous hydrochloric acid (37%, 3.75 vol) was added and stirring begun. During dissolution of the solid alcohol, a modest endotherm (5-6° C.) was observed. The resulting mixture was stirred overnight (16 h), slowly becoming dark red. A 30 L jacketed vessel was charged with water (5 vol) which was then cooled to 10° C. The reaction mixture was transferred slowly into the water by vacuum, maintaining the internal temperature of the mixture below 25° C. Hexanes (3 vol) was added and the resulting mixture was stirred for 0.5 h. The phases were settled and the aqueous phase (pH<1) was drained off and discarded. The organic phase was concentrated in vacuo using a rotary evaporator, furnishing the product as red oil.

### (4-(Benzyloxy)-3,3-dimethylbut-1-ynyl)trimethylsilane

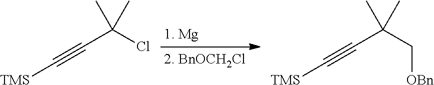

Method A

All equivalent and volume descriptors in this part are based on a 250 g reaction. Magnesium turnings (69.5 g, 2.86 mol, 2.0 equiv) were charged to a 3 L 4-neck reactor and stirred with a magnetic stirrer under nitrogen for 0.5 h. The reactor was immersed in an ice-water bath. A solution of the propargyl chloride (250 g, 1.43 mol, 1.0 equiv) in THF (1.8 L, 7.2 vol) was added slowly to the reactor, with stirring, until an initial exotherm (about 10° C.) was observed. The Grignard reagent formation was confirmed by IPC using 1H-NMR spectroscopy. Once the exotherm subsided, the remainder of the solution was added slowly, maintaining the batch temperature<15° C. The addition required about 3.5 h. The resulting dark green mixture was decanted into a 2 L capped bottle.

All equivalent and volume descriptors in this part are based on a 500 g reaction. A 22 L reactor was charged with a solution of benzyl chloromethyl ether (95%, 375 g, 2.31 mol, 0.8 equiv) in THF (1.5 L, 3 vol). The reactor was cooled in an ice-water bath. Two of the four Grignard reagent batches prepared above were combined and then added slowly to the benzyl chloromethyl ether solution via an addition funnel, maintaining the batch temperature below 25° C. The addition required 1.5 h. The reaction mixture was stirred overnight (16 h).

All equivalent and volume descriptors in this part are based on a 1 kg reaction. A solution of 15% ammonium chloride was prepared in a 30 L jacketed reactor (1.5 kg in 8.5 kg of water, 10 vol). The solution was cooled to 5° C. The two Grignard reaction mixtures above were combined and then transferred into the ammonium chloride solution via a header vessel. An exotherm was observed in this quench, which was carried out at a rate such as to keep the internal temperature below 25° C. Once the transfer was complete, the vessel jacket temperature was set to 25° C. Hexanes (8 L, 8 vol) was added and the mixture was stirred for 0.5 h. After settling the phases, the aqueous phase (pH 9) was drained off and discarded. The remaining organic phase was washed with water (2 L, 2 vol). The organic phase was concentrated in vacuo using a 22 L rotary evaporator, providing the crude product as an orange oil.

Method B

Magnesium turnings (106 g, 4.35 mol, 1.0 eq) were charged to a 22 L reactor and then suspended in THF (760 mL, 1 vol). The vessel was cooled in an ice-water bath such that the batch temperature reached 2° C. A solution of the propargyl chloride (760 g, 4.35 mol, 1.0 equiv) in THF (4.5 L, 6 vol) was added slowly to the reactor. After 100 mL was added, the addition was stopped and the mixture stirred until a 13° C. exotherm was observed, indicating the Grignard reagent initiation. Once the exotherm subsided, another 500 mL of the propargyl chloride solution was added slowly, maintaining the batch temperature <20° C. The Grignard reagent formation was confirmed by IPC using 1H-NMR spectroscopy. The remainder of the propargyl chloride solution was added slowly, maintaining the batch temperature <20° C. The addition required about 1.5 h. The resulting dark green solution was stirred for 0.5 h. The Grignard reagent formation was confirmed by IPC using ¹H-NMR spectroscopy. Neat benzyl chloromethyl ether was charged to the reactor addition funnel and then added dropwise into the reactor, maintaining the batch temperature below 25° C. The addition required 1.0 h. The reaction mixture was stirred overnight. The aqueous work-up and concentration was carried out using the same procedure and relative amounts of materials as in Method A to give the product as an orange oil.

### Benzyloxy-3,3-dimethylbut-1-yne

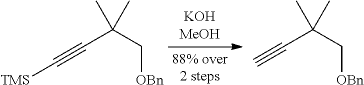 [SMILES: CC(C)(C#C[Si](C)(C)C)COCc1ccccc1 ; C#CC(C)(C)COCc1ccccc1]

A 30 L jacketed reactor was charged with methanol (6 vol) which was then cooled to 5° C. Potassium hydroxide (85%, 1.3 equiv) was added to the reactor. A 15-20° C. exotherm was observed as the potassium hydroxide dissolved. The jacket temperature was set to 25° C. A solution of 4-benzyloxy-3,3-dimethyl-1-trimethylsilylbut-1-yne (1.0 equiv) in methanol (2 vol) was added and the resulting mixture was stirred until reaction completion, as monitored by HPLC. Typical reaction time at 25° C. was 3-4 h. The reaction mixture was diluted with water (8 vol) and then stirred for 0.5 h. Hexanes (6 vol) was added and the resulting mixture was stirred for 0.5 h. The phases were allowed to settle and then the aqueous phase (pH 10-11) was drained off and discarded. The organic phase was washed with a solution of KOH (85%, 0.4 equiv) in water (8 vol) followed by water (8 vol). The organic phase was then concentrated down using a rotary evaporator, yielding the title material as a yellow-orange oil. Typical purity of this material was in the 80% range with primarily a single impurity present. 1H NMR (400 MHz, C6D6) δ 7.28 (d, 2H, J=7.4 Hz), 7.18 (t, 2H, J=7.2 Hz), 7.10 (d, 1H, J=7.2 Hz), 4.35 (s, 2H), 3.24 (s, 2H), 1.91 (s, 1H), 1.25 (s, 6H).

### Benzylglycolated 4-Amino-2-(4-benzyloxy-3,3-dimethylbut-1-ynyl)-5-fluoroaniline

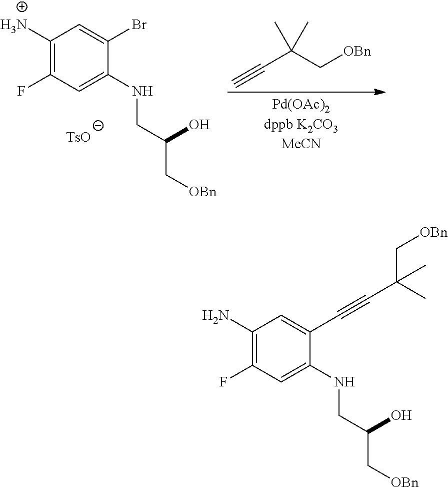 [SMILES: [NH3+]c1cc(Br)c(NC[C@@H](O)COCc2ccccc2)cc1F ; C#CC(C)(C)COCc1ccccc1 ; CC(C)(C#Cc1cc(N)c(F)cc1NC[C@@H](O)COCc1ccccc1)COCc1ccccc1]

Benzylglocolated 4-ammonium-2-bromo-5-fluoroaniline tosylate salt was freebased by stirring the solid in EtOAc (5 vol) and saturated NaHCO3 solution (5 vol) until a clear organic layer was achieved. The resulting layers were separated and the organic layer was washed with saturated NaHCO₃ solution (5 vol) followed by brine and concentrated in vacuo to obtain benzylglocolated 4-ammonium-2-bromo-5-fluoroaniline tosylate salt as an oil.

Then, a flask was charged with benzylglocolated 4-ammonium-2-bromo-5-fluoroaniline tosylate salt (freebase, 1.0 equiv), Pd(OAc) (4.0 mol %), dppb (6.0 mol %) and powdered K₂CO₃ (3.0 equiv) and stirred with acetonitrile (6 vol) at room temperature. The resulting reaction mixture was degassed for approximately 30 min by bubbling in N₂ with vent. Then 4-benzyloxy-3,3-dimethylbut-1-yne (1.1 equiv) dissolved in acetonitrile (2 vol) was added in a fast stream and heated to 80° C. and stirred until complete consumption of 4-ammonium-2-bromo-5-fluoroaniline tosylate salt was achieved. The reaction slurry was cooled to room temperature and filtered through a pad of Celite® and washed with acetonitrile (2 vol). Filtrate was concentrated in vacuo and the residue was redissolved in EtOAc (6 vol). The organic layer was washed twice with NH4Cl solution (20% w/v, 4 vol) and brine (6 vol). The resulting organic layer was concentrated to yield brown oil and used as is in the next reaction.

### N-Benzylglycolated-5-amino-2-(2-benzyloxy-1,1-dimethylethyl)-6-fluoroindole

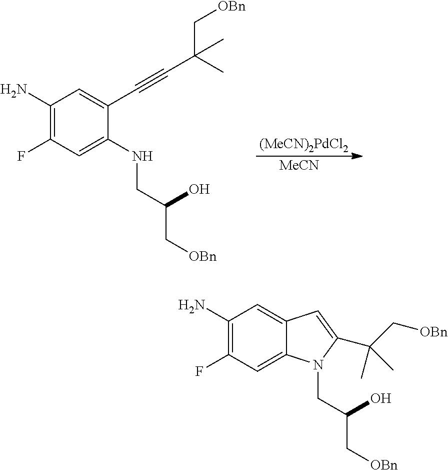 [SMILES: "(MeCN)2PdCl2" ; "MeCN" ; CC(C)(C#Cc1cc(N)c(F)cc1NC[C@@H](O)COCc1ccccc1)COCc1ccccc1 ; CC(C)(COCc1ccccc1)c1cc2cc(N)c(F)cc2n1C[C@@H](O)COCc1ccccc1]

Crude oil of benzylglycolated 4-amino-2-(4-benzyloxy-3,3-dimethylbut-1-ynyl)-5-fluoroaniline was dissolved in acetonitrile (6 vol) and added (MeCN)2PdCl2 (15 mol %) at room temperature. The resulting mixture was degassed using N2 with vent for approximately 30 min. Then the reaction mixture was stirred at 80° C. under N2 blanket overnight. The reaction mixture was cooled to room temperature and filtered through a pad of Celite® and washed the cake with acetonitrile (1 vol). The resulting filtrate was concentrated in vacuo and redissolved in EtOAc (5 vol). Deloxan-II® THP (5 wt % based on the theoretical yield of N-benzylglycolated-5-amino-2-(2-benzyloxy-1,1-dimethylethyl)-6-fluoroindole) was added and stirred at room temperature overnight. The mixture was then filtered through a pad of silica (2.5 inch depth, 6 inch diameter filter) and washed with EtOAc (4 vol). The filtrate was concentrated down to a dark brown residue, and used as is in the next reaction.

### Repurification of crude N-benzylglycolated-5-amino-2-(2-benzyloxy-1,1-dimethylethyl)-6-fluoroindole

The crude N-benzylglycolated-5-amino-2-(2-benzyloxy-1,1-dimethylethyl)-6-fluoroindole was dissolved in dichloromethane (about 1.5 vol) and filtered through a pad of silica initially using 30% EtOAc/heptane where impurities were discarded. Then the silica pad was washed with 50% EtOAc/heptane to isolate N-benzylglycolated-5-amino-2-(2-benzyloxy-1,1-dimethylethyl)-6-fluoroindole until faint color was observed in the filtrate. This filtrate was concentrated in vacuo to afford brown oil which crystallized on standing at room temperature. 1H NMR (400 MHz, DMSO) δ 7.38-7.34 (m, 4H), 7.32-7.23 (m, 6H), 7.21 (d, 1H, J=12.8 Hz), 6.77 (d, 1H, J=9.0 Hz), 6.06 (s, 1H), 5.13 (d, 1H, J=4.9 Hz), 4.54 (s, 2H), 4.46 (br. s, 2H), 4.45 (s, 2H), 4.33 (d, 1H, J=12.4 Hz), 4.09-4.04 (m, 2H), 3.63 (d, 1H, J=9.2 Hz), 3.56 (d, 1H, J=9.2 Hz), 3.49 (dd, 1H, J=9.8, 4.4 Hz), 3.43 (dd, 1H, J=9.8, 5.7 Hz), 1.40 (s, 6H).

### Synthesis of Compound 3

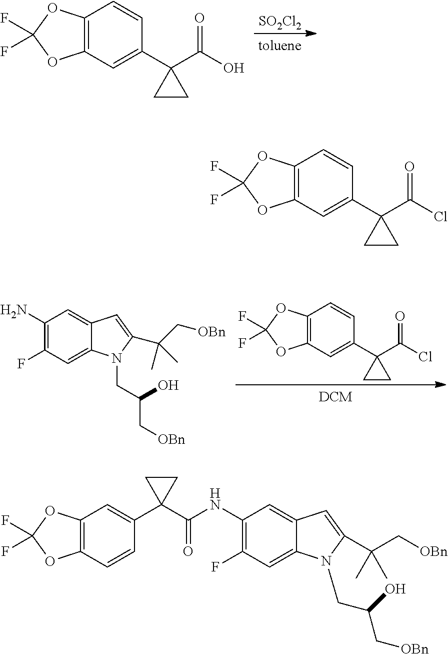

1-(2,2-Difluoro-1,3-benzodioxol-5-yl)-cyclopropanecarboxylic acid (1.3 equiv) was slurried in toluene (2.5 vol, based on 1-(2,2-difluoro-1,3-benzodioxol-5-yl)-cyclopropanecarboxylic acid) and the mixture was heated to 60° C. SOCl2 (1.7 equiv) was added via addition funnel. The resulting mixture was stirred for 2 h. The toluene and the excess SOCl2 were distilled off using rotavop. Additional toluene (2.5 vol, based on 1-(2,2-difluoro-1,3-benzodioxol-5-yl)-cyclopropanecarboxylic acid) was added and distilled again. The crude acid chloride was dissolved in dichloromethane (2 vol) and added via addition funnel to a mixture of N-benzylglycolated-5-amino-2-(2-benzyloxy-1,1-dimethylethyl)-6-fluoroindole (1.0 equiv), and triethylamine (2.0 equiv) in dichloromethane (7 vol) while maintaining 0-3° C. (internal temperature). The resulting mixture was stirred at 0° C. for 4 h and then warmed to room temperature overnight. Distilled water (5 vol) was added to the reaction mixture and stirred for no less than 30 min and the layers were separated. The organic phase was washed with 20 wt % K2CO3 (4 vol x 2) followed by a brine wash (4 vol) and concentrated to afford crude benzyl protected Compound 2 as a thick brown oil, which was purified further using silica pad filtration.

Silica gel pad filtration: Crude benzyl protected Compound 3 was dissolved in ethyl acetate (3 vol) in the presence of activated carbon Darco-G (10 wt %, based on theoretical yield of benzyl protected Compound 3) and stirred at room temperature overnight. To this mixture was added heptane (3 vol) and filtered through a pad of silica gel (2× weight of crude benzyl protected Compound 3). The silica pad was washed with ethyl acetate/heptane (1:1, 6 vol) or until little color was detected in the filtrate. The filtrate was concentrated in vacuo to afford benzyl protected Compound 3 as viscous reddish brown oil, and used directly in the next step.

Repurification: Benzyl protected Compound 3 was redissolved in dichloromethane (1 vol, based on theoretical yield of benzyl protected Compound 3) and loaded onto a silica gel pad (2× weight of crude benzyl protected Compound 3). The silica pad was washed with dichloromethane (2 vol, based on theoretical yield of benzyl protected Compound 3) and the filtrate was discarded. The silica pad was washed with 30% ethyl acetate/heptane (5 vol) and the filtrate was concentrated in vacuo to afford benzyl protected Compound 3 as viscous reddish orange oil, and used directly in the next step.

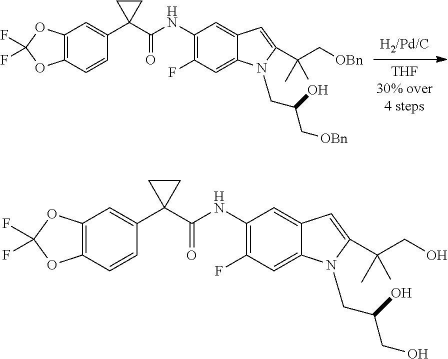 [SMILES: CC(C)(COCc1ccccc1)c1cc2cc(NC(=O)C3(c4ccc5c(c4)OC(F)(F)O5)CC3)c(F)cc2n1C[C@@H](O)COCc1ccccc1 ; CC(C)(CO)c1cc2cc(NC(=O)C3(c4ccc5c(c4)OC(F)(F)O5)CC3)c(F)cc2n1C[C@@H](O)CO]

Method A

A 20 L autoclave was flushed three times with nitrogen gas and then charged with palladium on carbon (Evonik E 101 NN/W, 5% Pd, 60% wet, 200 g, 0.075 mol, 0.04 equiv). The autoclave was then flushed with nitrogen three times. A solution of crude benzyl protected Compound 3 (1.3 kg, about 1.9 mol) in THF (8 L, 6 vol) was added to the autoclave via suction. The vessel was capped and then flushed three times with nitrogen gas. With gentle stirring, the vessel was flushed three times with hydrogen gas, evacuating to atmosphere by diluting with nitrogen. The autoclave was pressurized to 3 Bar with hydrogen and the agitation rate was increased to 800 rpm. Rapid hydrogen uptake was observed (dissolution). Once uptake subsided, the vessel was heated to 50° C.

For safety purposes, the thermostat was shut off at the end of every work-day. The vessel was pressurized to 4 Bar with hydrogen and then isolated from the hydrogen tank.

After 2 full days of reaction, more Pd/C (60 g, 0.023 mol, 0.01 equiv) was added to the mixture. This was done by flushing three times with nitrogen gas and then adding the catalyst through the solids addition port. Resuming the reaction was done as before. After 4 full days, the reaction was deemed complete by HPLC by the disappearance of not only the starting material, but also the peak corresponding to a mono-benzylated intermediate.

The reaction mixture was filtered through a Celite® pad. The vessel and filter cake were washed with THF (2 L, 1.5 vol). The Celite® pad was then wetted with water and the cake discarded appropriately. The combined filtrate and THF wash were concentrated using a rotary evaporator yielding the crude product as a black oil, 1 kg.

The equivalents and volumes in the following purification are based on 1 kg of crude material. The crude black oil was dissolved in 1:1 ethyl acetate-heptane. The mixture was charged to a pad of silica gel (1.5 kg, 1.5 wt. equiv) in a flitted funnel that had been saturated with 1:1 ethyl acetate-heptane. The silica pad was flushed first with 1:1 ethyl acetate-heptane (6 L, 6 vol) and then with pure ethyl acetate (14 L, 14 vol). The eluent was collected in 4 fractions that were analyzed by HPLC.

The equivalents and volumes in the following purification are based on 0.6 kg of crude material. Fraction 3 was concentrated by rotary evaporation to give a brown foam (600 g) and then redissolved in MTBE (1.8 L, 3 vol). The dark brown solution was stirred overnight at ambient temperature, during which time, crystallization occurred. Heptane (55 mL, 0.1 vol) was added and the mixture was stirred overnight. The mixture was filtered using a Buchner funnel and the filter cake was washed with 3:1 MTBE-heptane (900 mL, 1.5 vol). The filter cake was air-dried for 1 h and then vacuum dried at ambient temperature for 16 h, furnishing 253 g of Compound 3 as an off-white solid.

The equivalents and volumes for the following purification are based on 1.4 kg of crude material. Fractions 2 and 3 from the above silica gel filtration as well as material from a previous reaction were combined and concentrated to give 1.4 kg of a black oil. The mixture was resubmitted to the silica gel filtration (1.5 kg of silica gel, eluted with 3.5 L, 2.3 vol of 1:1 ethyl acetate-heptane then 9 L, 6 vol of pure ethyl acetate) described above, which upon concentration gave a tan foamy solid (390 g).

The equivalents and volumes for the following purification are based on 390 g of crude material. The tan solid was insoluble in MTBE, so was dissolved in methanol (1.2 L, 3 vol). Using a 4 L Morton reactor equipped with a long-path distillation head, the mixture was distilled down to 2 vol. MTBE (1.2 L, 3 vol) was added and the mixture was distilled back down to 2 vol. A second portion of MTBE (1.6 L, 4 vol) was added and the mixture was distilled back down to 2 vol. A third portion of MTBE (1.2 L, 3 vol) was added and the mixture was distilled back down to 3 vol. Analysis of the distillate by GC revealed it to consist of about 6% methanol. The thermostat was set to 48° C. (below the boiling temp of the MTBE-methanol azeotrope, which is 52° C.). The mixture was cooled to 20° C. over 2 h, during which time a relatively fast crystallization occurred. After stirring the mixture for 2 h, heptane (20 mL, 0.05 vol) was added and the mixture was stirred overnight (16 h). The mixture was filtered using a Buchner funnel and the filter cake was washed with 3:1 MTBE-heptane (800 mL, 2 vol). The filter cake was air-dried for 1 h and then vacuum dried at ambient temperature for 16 h, furnishing 130 g of Compound 3 as an off-white solid.

Method B

Benzyl protected Compound 3 was dissolved and flushed with THF (3 vol) to remove any remaining residual solvent. Benzyl protected Compound 3 was redissolved in THF (4 vol) and added to the hydrogenator containing 5 wt % Pd/C (2.5 mol %, 60% wet, Degussa E5 E101 NN/W). The internal temperature of the reaction was adjusted to 50° C., and flushed with N2 (×5) followed by hydrogen (×3). The hydrogenator pressure was adjusted to 3 Bar of hydrogen and the mixture was stirred rapidly (>1100 rpm). At the end of the reaction, the catalyst was filtered through a pad of Celite® and washed with THF (1 vol). The filtrate was concentrated in vacuo to obtain a brown foamy residue. The resulting residue was dissolved in MTBE (5 vol) and 0.5N HCl solution (2 vol) and distilled water (1 vol) were added. The mixture was stirred for no less than 30 min and the resulting layers were separated. The organic phase was washed with 10 wt % K₂CO₃ solution (2 vol×2) followed by a brine wash. The organic layer was added to a flask containing silica gel (25 wt %), Deloxan-II® THP (5 wt %, 75% wet), and Na2SO4 and stirred overnight. The resulting mixture was filtered through a pad of Celite® and washed with 10% THF/MTBE (3 vol). The filtrate was concentrated in vacuo to afford crude Compound 3 as a pale tan foam.

Recovery of Compound 3 Mother Liquor:

Option A.

Silica gel pad filtration: The mother liquor was concentrated in vacuo to obtain a brown foam, dissolved in dichloromethane (2 vol), and filtered through a pad of silica (3× weight of the crude Compound 3). The silica pad was washed with ethyl acetate/heptane (1:1, 13 vol) and the filtrate was discarded. The silica pad was washed with 10% THF/ethyl acetate (10 vol) and the filtrate was concentrated in vacuo to afford Compound 3 as pale tan foam. The above crystallization procedure was followed to isolate the remaining Compound 3.

Option B.

Silica gel column chromatography: After chromatography on silica gel (50% ethyl acetate/hexanes to 100% ethyl acetate), the desired compound was isolated as pale tan foam. The above crystallization procedure was followed to isolate the remaining Compound 3.

Compound 3 may also be prepared by one of several synthetic routes disclosed in US published patent application US 2009/0131492, incorporated herein by reference in its entirety.

TABLE II
D-4: Physical Data for Compound 3.

| Cmpd. | LC/MS | LC/RT | |
|---|---|---|---|
| No. | M + 1 | min | NMR |
| 3 | 521.5 | 1.69 | 1H NMR (400.0 MHz, CD3CN) d 7.69 (d, J = 7.7 Hz, 1H), |
| | | | 7.44 (d, J = 1.6 Hz, 1H), 7.39 (dd, J = 1.7, 8.3 Hz, 1H), 7.31 (s, |
| | | | 1H), 7.27 (d, J = 8.3 Hz, 1H), 7.20 (d, J = 12.0 Hz, 1H), 6.34 (s, |
| | | | 1H), 4.32 (d, J = 6.8 Hz, 2H), 4.15-4.09 (m, 1H), 3.89 (dd, J = |
| | | | 6.0, 11.5 Hz, 1H), 3.63-3.52 (m, 3H), 3.42 (d, J = 4.6 Hz, 1H), |
| | | | 3.21 (dd, J = 6.2, 7.2 Hz, 1H), 3.04 (t, J = 5.8 Hz, 1H), 1.59 (dd, |
| | | | J = 3.8, 6.8 Hz, 2H), 1.44 (s, 3H), 1.33 (s, 3H) and 1.18 (dd, J = |
| | | | 3.7, 6.8 Hz, 2H) ppm. |

### II.E Embodiments of Column E Compounds

### II.E.1 Embodiments of ENaC Compounds

The inhibitors of ENaC activity in Column E are fully described and exemplified in International Patent Application No. PCT/EP2008/067110 filed: Dec. 9, 2008 and is Assigned to Novartis AG. All of the compounds recited in PCT/EP2008/067110, are useful in the present invention and the compounds and methods for making such compounds are hereby incorporated into the present disclosure in their entirety.

Column E compounds (ENaC inhibitors) can also include the compounds of Formula E described below, and one or more of: camostat (a trypsin-like protease inhibitor), QAU145, 552-02, GS-9411, INO-4995, Aerolytic, amiloride, benzamil, dimethyl-amiloride, and ENaC inhibitor compounds disclosed in International Applications: PCT/EP2006/003387 filed Oct. 19, 2006; PCT/EP2006/012314 filed Jun. 28, 2007 and PCT/EP2006/012320 filed Jun. 28, 2007. All of these International Patent Application disclosures are hereby incorporated herein by reference in their entireties. In some embodiments, the ENaC inhibitor is amiloride. Methods for determining whether a compound is an ENaC inhibitor are known in the art and can be used to identify an ENaC inhibitor that can be used in the combination with CF modulator component described herein.

### II.E.2 ENaC Compounds of Formula E

The present invention is directed to pharmaceutical compositions comprising at least one ABC transporter modulator component as provided by Columns A-D in Table I and at least one ENaC inhibitor as provided in Column E of Table I. The invention also provides methods for treating CF and other chronic diseases, methods for preparing the compositions and methods for using the compositions for the treatment of CF and other chronic diseases, including chronic diseases involving regulation of fluid volumes across epithelial membranes, using compositions containing an ABC transporter modulator compound and ENaC inhibitor compounds. As uses herein, ENaC inhibitors can include the compounds of Formula E, including compounds of Formula E1.

In one aspect, the invention provides ENaC inhibitor compounds according to Formula E:

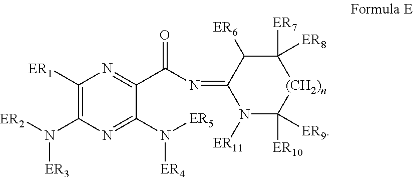

or solvates, hydrates or pharmaceutically acceptable salts thereof, wherein ER¹ is H, halogen, C₁-C₈-alkyl, C₁C₈-haloalkyl, C₁-C₈-haloalkoxy, C₃C₁₅-carbocyclic group, nitro, cyano, a C₆-C₁₅-membered aromatic carbocyclic group, or a C₁-C₈-alkyl substituted by a C₆-C₁₅-membered aromatic carbocyclic group;

ER², ER³, ER⁴ and ER⁵ are each independently selected from H and C₁-C₆ alkyl;

ER⁶, ER⁷, ER⁸, ER⁹, ER¹⁰ and ER¹¹ are each independently selected from H; SO₂ER¹⁶; aryl optionally substituted by one or more Z groups; a C₃-C₁₀ carbocyclic group optionally substituted by one or more Z groups; C₃-C₁₄ heterocyclic group optionally substituted by one or more Z groups; C₁-C₈ alkyl optionally substituted by an aryl group which is optionally substituted by one or more Z groups, a C₃-C₁₀ carbocyclic group optionally substituted by one or more Z groups or a C₃-C₁₄ heterocyclic group optionally substituted by one or more Z groups; or is represented by the Formula E2:

—(C₀-C₆ alkylene)-A-(C₀-C₆ alkylene)-B-(X-ER¹²)_{q}-ER²²,

wherein the alkylene groups are optionally substituted by one or more Z groups;

or ER⁶ and ER⁷ together with the atoms to which they are attached form a 3- to 10-membered heterocyclic group, the heterocyclic group including one or more further heteroatoms selected from N, O and S, and the heterocyclic group being optionally substituted by one or more Z groups; SO₂ER¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the formula 2;

or ER⁷ and ER⁸ together with the carbon atom to which they are attached form a 3- to 10-membered carbocyclic or a 3- to 10-membered heterocyclic group, the heterocyclic group including one or more heteroatoms selected from N, O and S, and the carbocyclic and heterocyclic groups being optionally substituted by one or more Z groups; SO₂R¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the formula 2;

or ER⁹ and ER¹⁰ together with the carbon atom to which they are attached form a 3- to 10-membered carbocyclic or a 3- to 10-membered heterocyclic group, the heterocyclic group including one or more heteroatoms selected from N, O and S, and the carbocyclic and heterocyclic groups being optionally substituted by one or more Z groups; SO₂ER¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the Formula E2;

or ER⁸ and ER⁹ together with the carbon atoms to which they are attached form a 3- to 10-membered cycloalkyl or a 3- to 10-membered heterocyclic group, the heterocyclic group including one or more heteroatoms selected from N, O and S, and the carbocyclic and heterocyclic groups being optionally substituted by one or more Z groups; SO₂ER¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the formula 2;

or ER¹⁰ and ER¹¹ together with the atoms to which they are attached form a 3- to 10-membered heterocyclic group, the heterocyclic group including one or more further heteroatoms selected from N, O and S, and the heterocyclic group being optionally substituted by one or more Z groups; SO₂ER¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the formula 2;

A is selected from a bond, —NER¹³(SO₂)—, —(SO₂)NER¹³—, —(SO₂)—, —NER¹³C(O)—, —C(O)NER¹³—, —NER¹³C(O)NER¹⁴—, —NER¹³C(O)O—, —NER¹³—, C(O)O, OC(O), C(O), O and S;

B is selected from a bond, —(C₂-C₄ alkenyl group)-, —(C₂-C₄ alkynyl group)-, —NH—, aryl, O-aryl, NH-aryl, a C₃-C₁₄ carbocyclic group and a 3- to 14-membered heterocyclic group, the heterocyclic group including one or more heteroatoms selected from N, O and S, wherein the aryl, carbocyclic and heterocyclic groups are each optionally substituted by one or more Z groups;

X is selected from a bond, —NER¹⁵(SO₂)—, —(SO₂)NER¹⁵—, —(SO₂)—, —NER¹⁵C(O)—, —C(O)NER¹⁵—, —NER¹⁵C(O)NER¹⁷—, —NER¹⁵C(O)O—, —NER¹⁵—, C(O)O, OC(O), C(O), O and S;

ER¹² is selected from C₁-C₈ alkylene, C₁-C₈ alkenylene, —C₃-C₈ cycloalkyl-, —C₁-C₈ alkylene-C₃-C₈ cycloalkyl-, and -aryl-, wherein the alkylene, cycloalkyl and aryl groups are optionally substituted by one or more Z groups;

ER¹³, ER¹⁴, ER¹⁵ and ER¹⁷ are each independently selected from H and C₁-C₆ alkyl;

ER¹⁶ is selected from C₁-C₈ alkyl, aryl and a 3- to 14-membered heterocyclic group, the heterocyclic group including one or more heteroatoms selected from N, O and S;

Z is independently selected from OH, aryl, O-aryl, C₇-C₁₄ aralkyl, O—C₇-C₁₄ aralkyl, C₁-C₆ alkyl, C₁-C₆ alkoxy, NER¹⁹(SO₂)ER²¹, (SO₂)NER¹⁹ER²¹, (SO₂)ER²⁰, NER¹⁹C(O)ER²⁰, C(O)NER¹⁹ER²⁰, NER¹⁹C(O)NER²⁰ER¹⁸, NER¹⁹C(O)OER²⁰, NER¹⁹ER²¹, C(O)OER¹⁹, C(O)ER¹⁹, SER¹⁹, OER¹⁹, oxo, CN, NO₂, and halogen, wherein the alkyl, alkoxy, aralkyl and aryl groups are each optionally substituted by one or more substituents selected from OH, halogen, C₁-C₄ haloalkyl and C₁-C₄ alkoxy;

ER¹⁸ and ER²⁰ are each independently selected from H and C₁-C₆ alkyl;

ER¹⁹ and ER²¹ are each independently selected from H; C₁-C₈ alkyl; C₃-C₈ cycloalkyl; C₁-C₄ alkoxy-C₁-C₆ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl and C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

ER¹⁹ and ER²⁰ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy;

ER²² is selected from H, halogen, C₁-C₈ alkyl, C₁-C₈ alkoxy, aryl, O-aryl, S(O)₂-aryl, S(O)2-C₁-C₆ alkyl, S(O)₂NER²³ER²⁴, NHS(O)₂NER²³ER²⁴, a C₃-C₁₄ carbocyclic group, a 3- to 14-membered heterocyclic group, the heterocyclic group including one or more heteroatoms selected from N, O and S, and O-(3- to 14-membered heterocyclic group, the heterocyclic group including one or more heteroatoms selected from N, O and S), wherein the alkyl, aryl, carbocyclic and heterocyclic groups are each optionally substituted by one or more Z groups;

ER²³ and ER²⁴ are each independently selected from H, C₁-C₈ alkyl and C₃-C₈ cycloalkyl; or

ER²³ and ER²⁴ together with the nitrogen atom to which they are attached form a 5- to 10-membered heterocyclic group, optionally including one or more further heteroatoms selected from N, O and S, wherein the heterocyclic group is optionally substituted by one or more Z groups;

n is 0, 1 or 2;

and p are each independently an integer from 0 to 6; and

q is 0, 1, 2 or 3;

with the proviso that when n is 0, at least one of ER⁶, ER⁷, ER⁸, ER⁹, ER¹⁰ and ER¹¹ is other than H.

In an embodiment of the invention, there is provided a compound according to the Formula Ea:

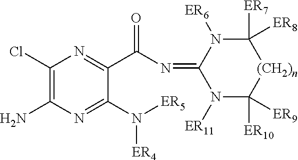

wherein

ER⁶, ER⁷, ER⁸, ER⁹, ER¹⁰ and ER¹¹ are each independently selected from H; SO₂ER¹⁶; aryl optionally substituted by one or more Z groups; a C₃-C₁₀ carbocyclic group optionally substituted by one or more Z groups; C₃-C₁₄ heterocyclic group optionally substituted by one or more Z groups; C₁-C₈ alkyl optionally substituted by an aryl group, a C₃-C₁₀ carbocyclic group optionally substituted by one or more Z groups or a C₃-C₁₄ heterocyclic group optionally substituted by one or more Z groups; or is represented by the Formula E2a:

-(CH₂)ₒ-A-(CH₂)ₚ—B—(X-ER¹²)_{q}-ER²²;

or ER⁷ and ER⁸ together with the carbon atom to which they are attached form a 3- to 7-membered carbocyclic or a 3- to 7-membered heterocyclic group, the heterocyclic group including one or more heteroatoms selected from N, O and S, and the carbocyclic and heterocyclic groups being optionally substituted by one or more Z groups; SO₂ER¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the Formula E2a;

or ER⁹ and E¹⁰ together with the carbon atom to which they are attached form a 3- to 7-membered carbocyclic or a 3- to 7-membered heterocyclic group, the heterocyclic group including one or more heteroatoms selected from N, O and S, and the carbocyclic and heterocyclic groups being optionally substituted by one or more Z groups; SO₂ER¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the Formula E2a;

or ER⁸ and ER⁹ together with the carbon atoms to which they are attached form a 3- to 7-membered cycloalkyl or a 3- to 7-membered heterocyclic group, the heterocyclic group including one or more heteroatoms selected from N, O and S, and the carbocyclic and heterocyclic groups being optionally substituted by one or more Z groups; SO₂ER¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the Formula Eta;

A is selected from a bond, —NER¹³(SO₂)—, —(SO₂)NER¹³—, —(SO₂)—, —NER¹³C(O)—, —(O)NER¹³, —NER¹³C(O)NER¹⁴—, —NER¹³C(O)O—, —NER¹³—, C(O)O, OC(O), C(O), O and S;

B is selected from a bond, aryl, a C₃-C₁₄ carbocyclic group and a C₃-C₁₄ heterocyclic group, wherein the ring systems are optionally substituted by one or more Z groups;

X is selected from a bond, —NER¹⁵(SO₂)—, —(SO₂)NER¹⁵—, —(SO₂)—, —NER¹⁵C(O)—, —C(O)NER¹⁵—, —NER¹⁵C(O)NER¹⁷—, —NER¹⁵C(O)O—, —NER¹⁵—, C(O)O, OC(O), C(O), O and S;

ER¹² is selected from H, C₁-C₈ alkyl, C₃-C₈ cycloalkyl, C₁-C₈ alkyl-C₃-C₈ cycloalkyl, C₁-C₈ alkyl-aryl and aryl, wherein the alkyl, cycloalkyl and aryl groups are optionally substituted by one or more Z groups;

ER¹³, ER¹⁴, ER¹⁵ and R¹⁷ are each independently selected from H and C₁-C₆ alkyl;

ER¹⁶ is selected from C₁-C₈ alkyl, aryl and a 3- to 14-membered heterocyclic group; Z is independently selected from OH, aryl, O-aryl, C₇-C₁₄ aralkyl, O—C₇-C₁₄ aralkyl, C₁-C₆ alkyl, C₁-C₆ alkoxy, NER¹⁹(SO₂)ER²¹, (SO₂)NER¹⁹ER²¹, (SO₂)ER²⁰, NER¹⁹C(O)ER²⁰, C(O)NER¹⁹ER²⁰, NER¹⁹C(O)NER²⁰ER¹⁸, NER¹⁹C(O)OER²⁰, NER¹⁹ER²¹, C(O)OER¹⁹, C(O)ER¹⁹, SER¹⁹, OER¹⁹, oxo, CN, NO₂, and halogen, wherein the alkyl, alkoxy, aralkyl and aryl groups are each optionally substituted by one or more substituents selected from OH, halogen, C₁-C₄ haloalkyl and C₁-C₄ alkoxy;

ER¹⁸, ER¹⁹ and ER²⁰ are each independently selected from H and C₁-C₆ alkyl; ER²¹ is selected from C₁-C₈ alkyl, aryl and a 3- to 14-membered heterocyclic group;

ER²² is selected from H and C₁-C₈ alkyl;

n is 0, 1 or 2;

and p are each independently an integer from 0 to 6; and

q is 0, 1, 2 or 3;

with the proviso that when n is 0, at least one of ER⁶, ER⁷, ER⁸, ER⁹, ER¹⁰ and ER¹¹ is other than H.

In a further embodiment of the invention as defined anywhere above, ER⁶ is selected from H, C₁-C₃ alkyl and (CH₂)_{d}-phenyl, where the phenyl group is optionally substituted by OER²³;

ER²³ is H or C₁-C₆ alkyl; and

d is an integer from 1 to 5 (optionally 2 to 4).

In a still further embodiment of the invention as defined anywhere above, ER⁷ is H or C₁-C₆; and

ER⁸ is selected from H, C₁-C₆ alkyl; (CH₂)ₑphenyl, where the phenyl group is optionally substituted by one or more groups selected from halo and OER²⁴; (CH₂)_{f}COOER²⁵; (CH₂)_{g}OC₁-C₆ alkyl, where the alkyl group is optionally substituted by 1 to 3 groups selected from OH, C₁-C₃ alkyl and phenyl; and (CH₂)ₕNHCO₂(CH₂)ᵢphenyl;

ER²⁴ is H or C₁-C₆ alkyl, where the alkyl group is optionally substituted by 1 to 3 groups selected from OH and OC₁-C₃ alkyl;

ER²⁵ is H or C₁-C₃ alkyl;

e is 0, 1, 2, 3, 4 or 5 (optionally 0, 1, 2, 3 or 4);

f, g and h are each independently an integer from 1 to 4; and

i is 1 or 2;

or ER⁷ and ER⁸ together with the carbon atom to which they attached form a 5- or 6-membered non-aromatic carbocyclic ring system or a 5- or 6-membered non-aromatic heterocyclic ring system containing one or more heteroatoms selected from N, O and S, the ring systems being optionally substituted by one or more Z groups; SO₂R¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the Formula E2 or E2a. Suitably, the ring system defined by ER⁷, ER⁸ and the carbon to which they are attached is optionally substituted by C₁-C₃ alkyl, halo or benzyl.

Optionally, f is 2 or 3. Additionally or alternatively, g may be 2 or 3. Additionally or alternatively, h may be 2, 3 or 4. Additionally or alternatively, i may be 1. In the immediately preceding sub-definitions off, g, h and i, each sub-definition may be combined with more other sub-definitions or they may be combined with the definitions for the relevant variables given above.

In a yet further embodiment of the invention as defined anywhere above, ER⁹ is H, C₁-C₆ alkyl or phenyl;

or R⁸ and R⁹ together with the carbon atoms to which they attached form a 5-, 6- or 7-membered non-aromatic carbocyclic ring system or a 5-, 6- or 7-membered non-aromatic heterocyclic ring system containing one or more heteroatoms selected from N, O and S, the ring systems being optionally substituted by C₁-C₃ alkyl, halo or benzyl.

In a further embodiment of the invention as defined anywhere above, R¹¹ is H, SO₂C₁-C₆ alkyl or SO₂phenyl.

In a further embodiment of the invention as defined anywhere above, R⁶ and R¹¹ are both H.

A further embodiment of the invention provides a compound according to the Formula Eb:

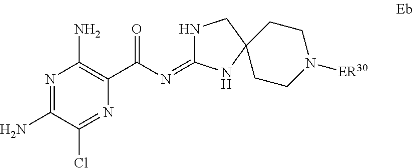

or the Formula Ec:

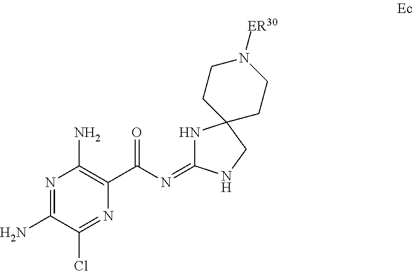

wherein ER³⁰ is -A-(C₀-C₆ alkylene)-B-(X-ER¹²)_{q}-ER²²

and A, B, X, ER¹², q and ER²² are as defined anywhere herein.

In a further aspect, of the embodiments of ENaC inhibitors, compounds of Formula E2 can include:

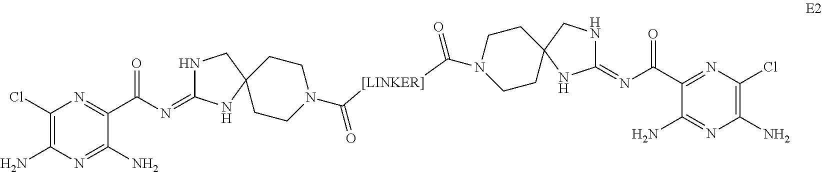

Exemplary compounds of Formula E include:

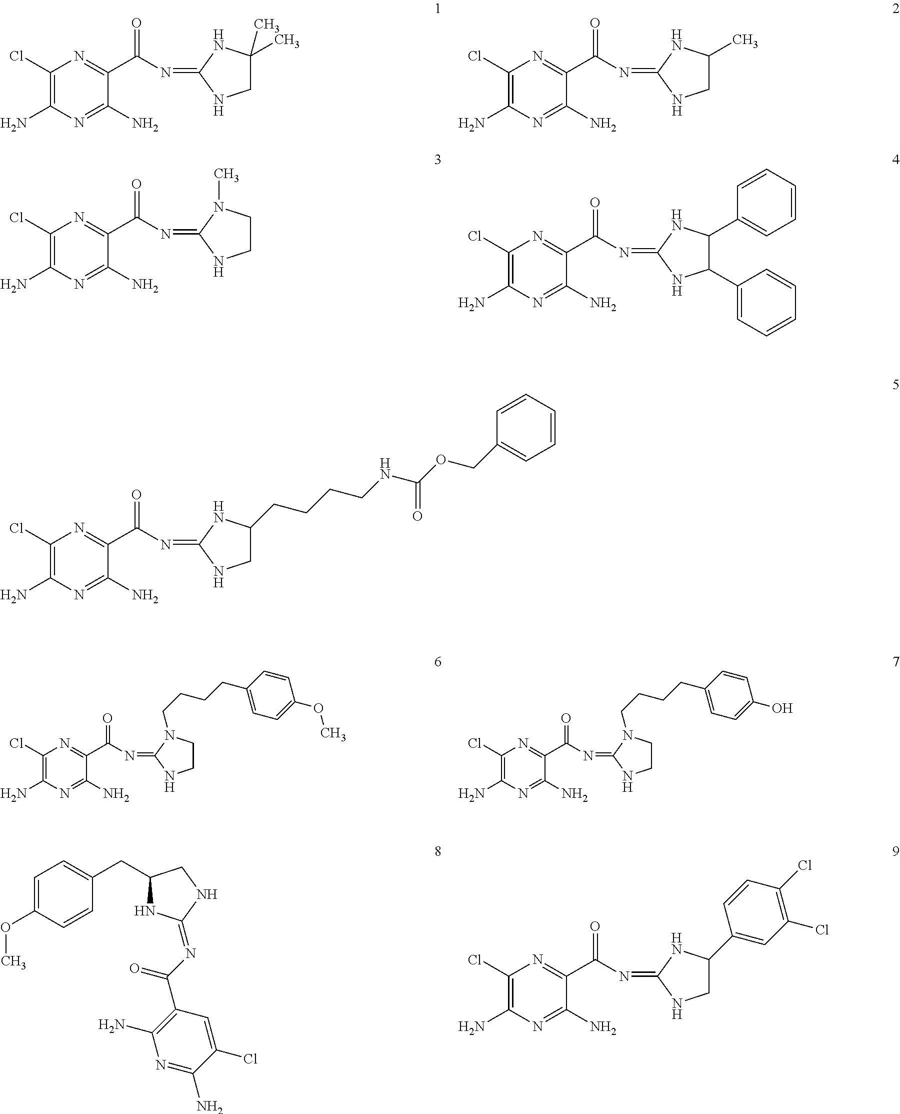

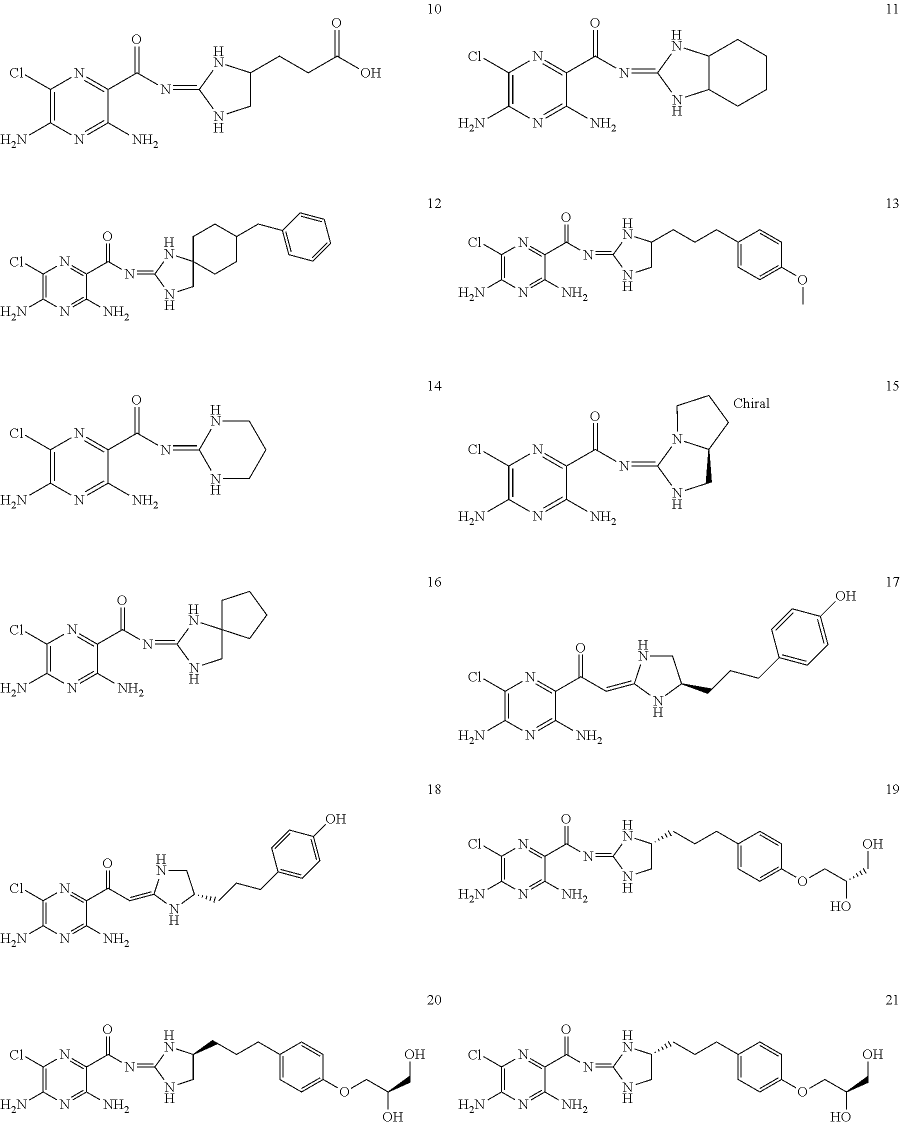

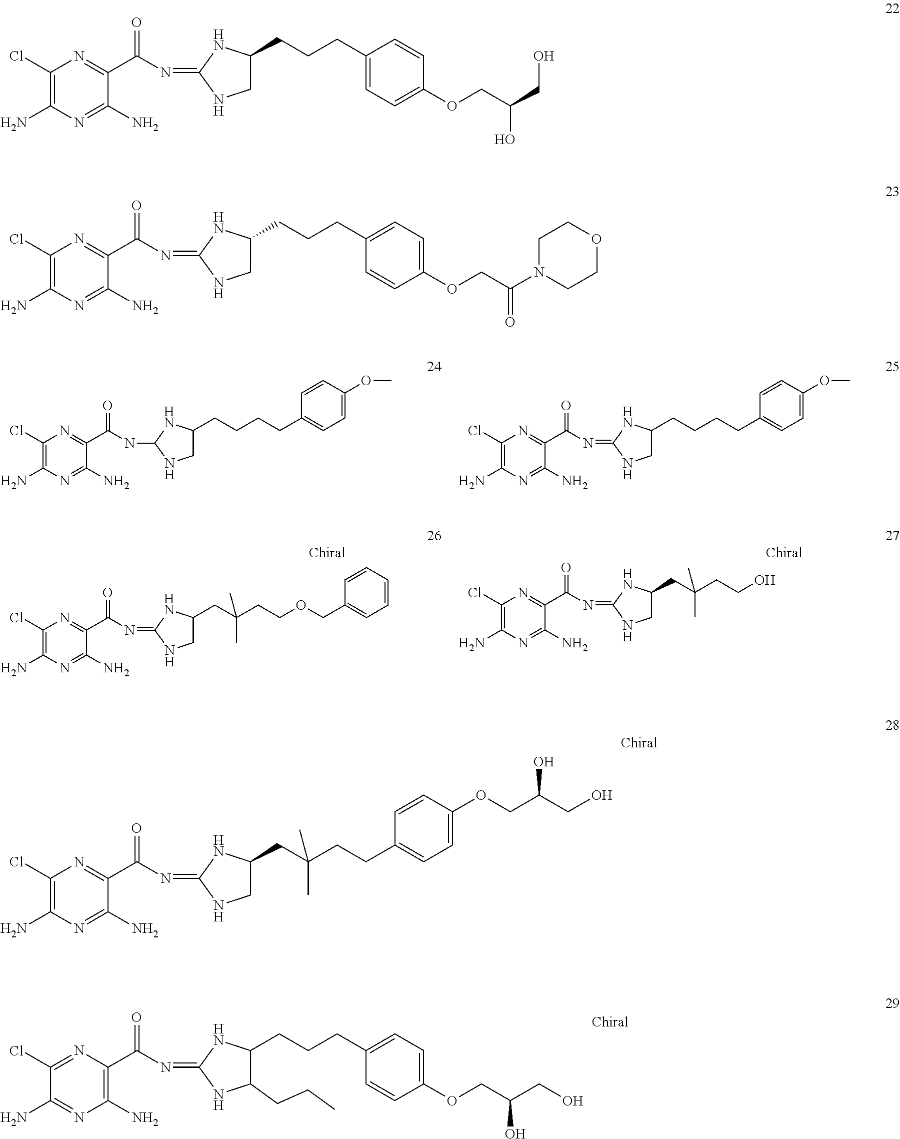

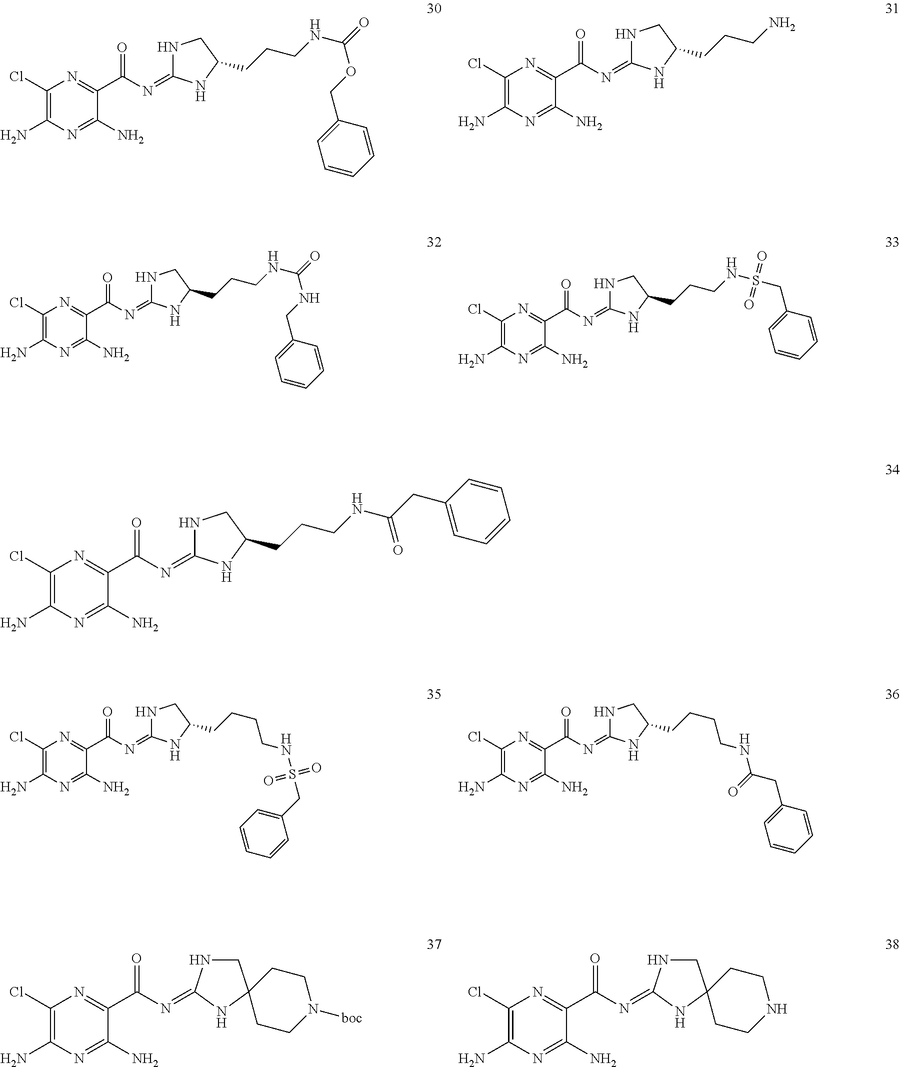

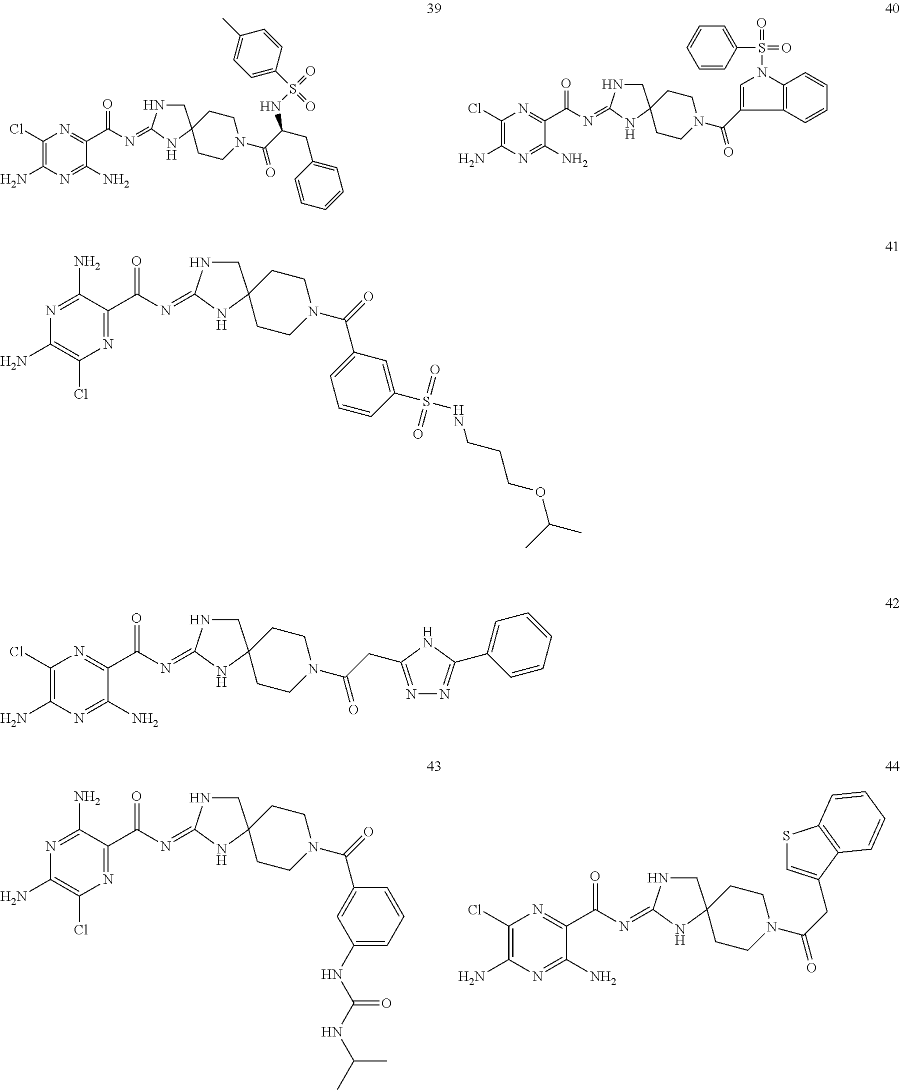 [SMILES: Cc1ccc(S(=O)(=O)N[C@@H](Cc2ccccc2)C(=O)N2CCC3(CC2)CN/C(=N\C(=O)c2nc(Cl)c(N)nc2N)N3)cc1 ; Nc1nc(N)c(C(=O)/N=C2\NCC3(CCN(C(=O)c4cn(S(=O)(=O)c5ccccc5)c5ccccc45)CC3)N2)nc1Cl ; CC(C)OCCCNS(=O)(=O)c1cccc(C(=O)N2CCC3(CC2)CN/C(=N\C(=O)c2nc(Cl)c(N)nc2N)N3)c1 ; Nc1nc(N)c(C(=O)/N=C2\NCC3(CCN(C(=O)Cc4nnc(-c5ccccc5)[nH]4)CC3)N2)nc1Cl ; CC(C)NC(=O)Nc1cccc(C(=O)N2CCC3(CC2)CN/C(=N\C(=O)c2nc(Cl)c(N)nc2N)N3)c1 ; Nc1nc(N)c(C(=O)/N=C2\NCC3(CCN(C(=O)Cc4csc5ccccc45)CC3)N2)nc1Cl]

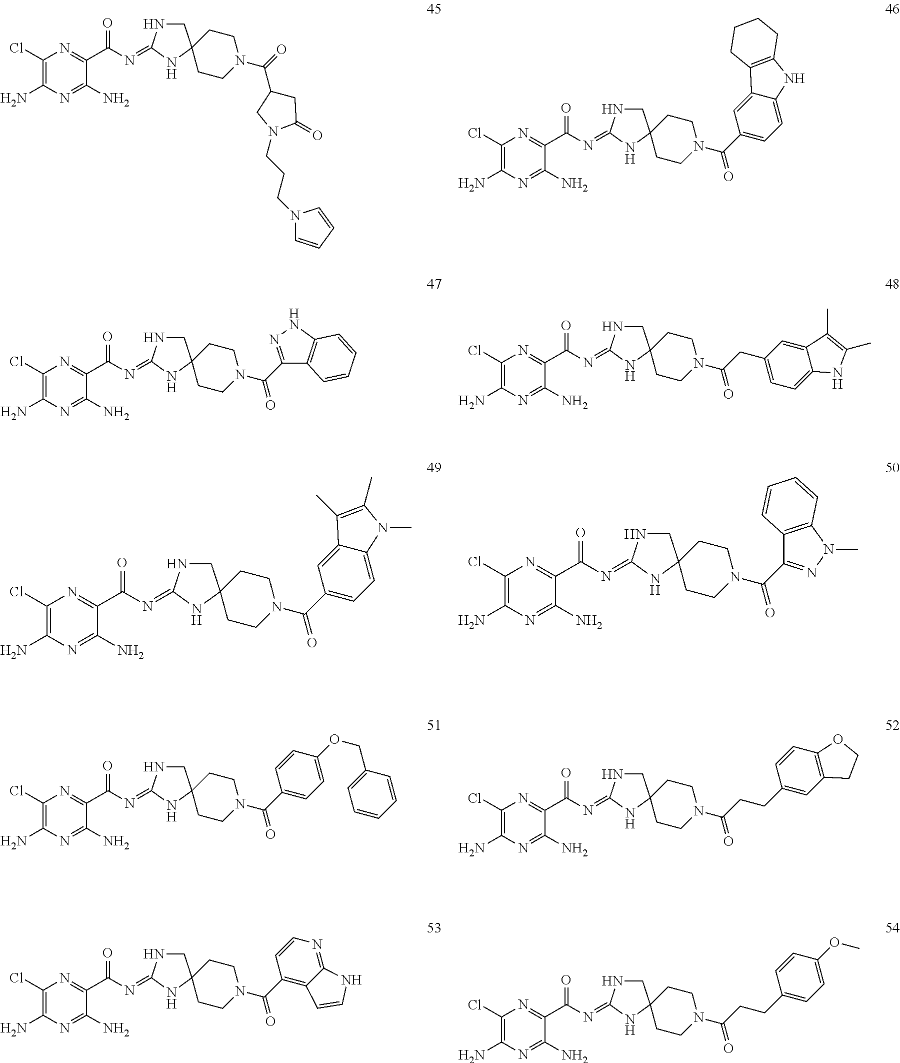

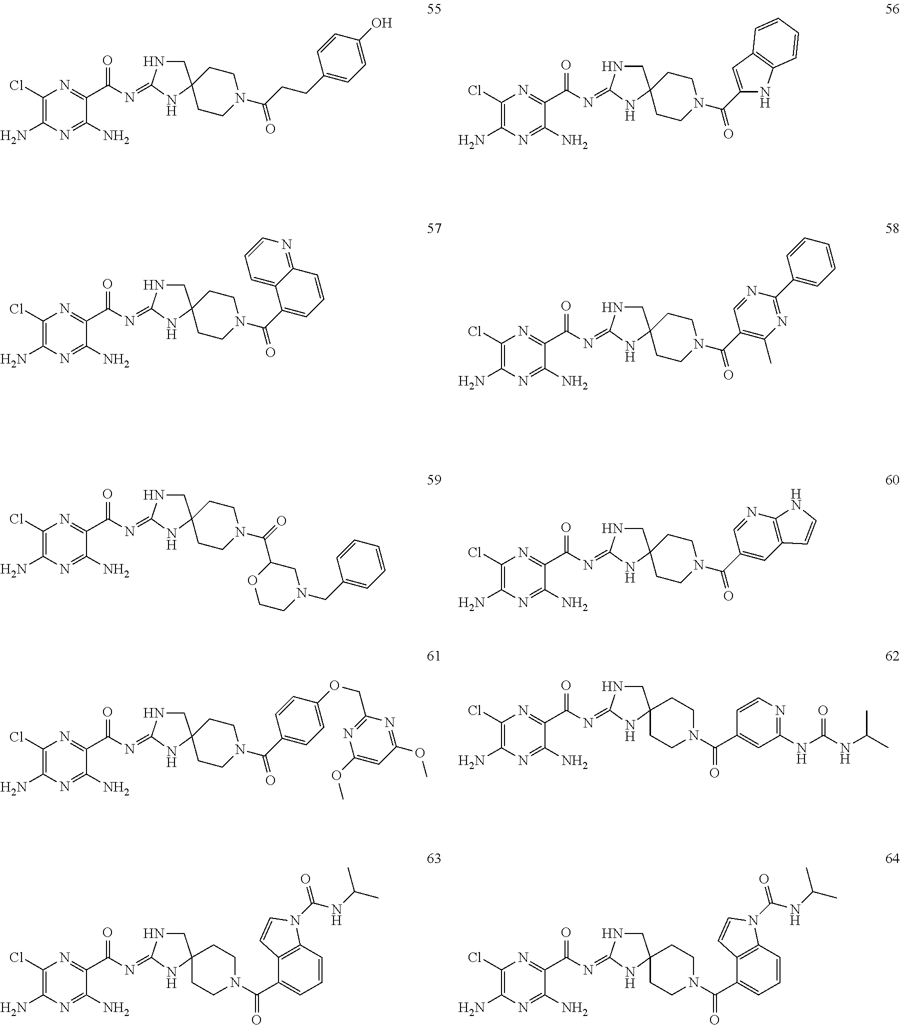

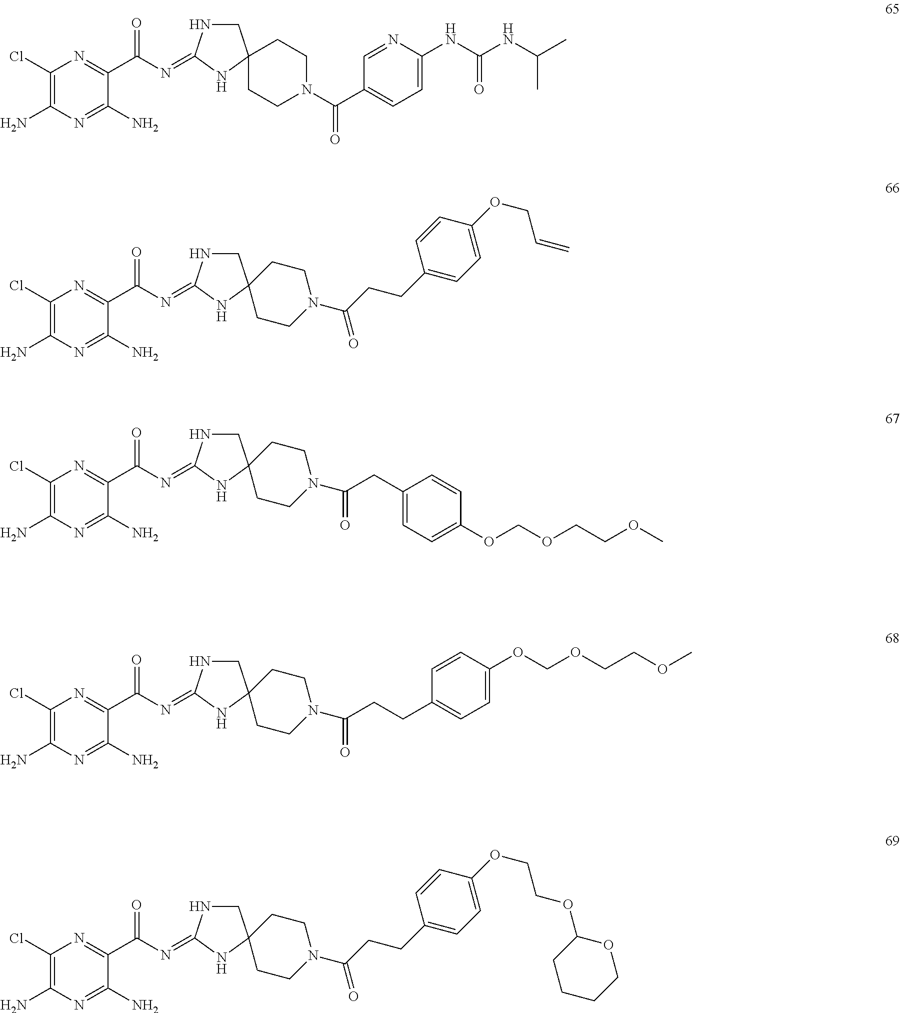 [SMILES: CC(C)NC(=O)Nc1ccc(C(=O)N2CCC3(CC2)CN/C(=N\C(=O)c2nc(Cl)c(N)nc2N)N3)cn1 ; C=CCOc1ccc(CCC(=O)N2CCC3(CC2)CN/C(=N\C(=O)c2nc(Cl)c(N)nc2N)N3)cc1 ; COCCOCOc1ccc(CC(=O)N2CCC3(CC2)CN/C(=N\C(=O)c2nc(Cl)c(N)nc2N)N3)cc1 ; COCCOCOc1ccc(CCC(=O)N2CCC3(CC2)CN/C(=N\C(=O)c2nc(Cl)c(N)nc2N)N3)cc1 ; Nc1nc(N)c(C(=O)/N=C2\NCC3(CCN(C(=O)CCc4ccc(OCCOC5CCCCO5)cc4)CC3)N2)nc1Cl]

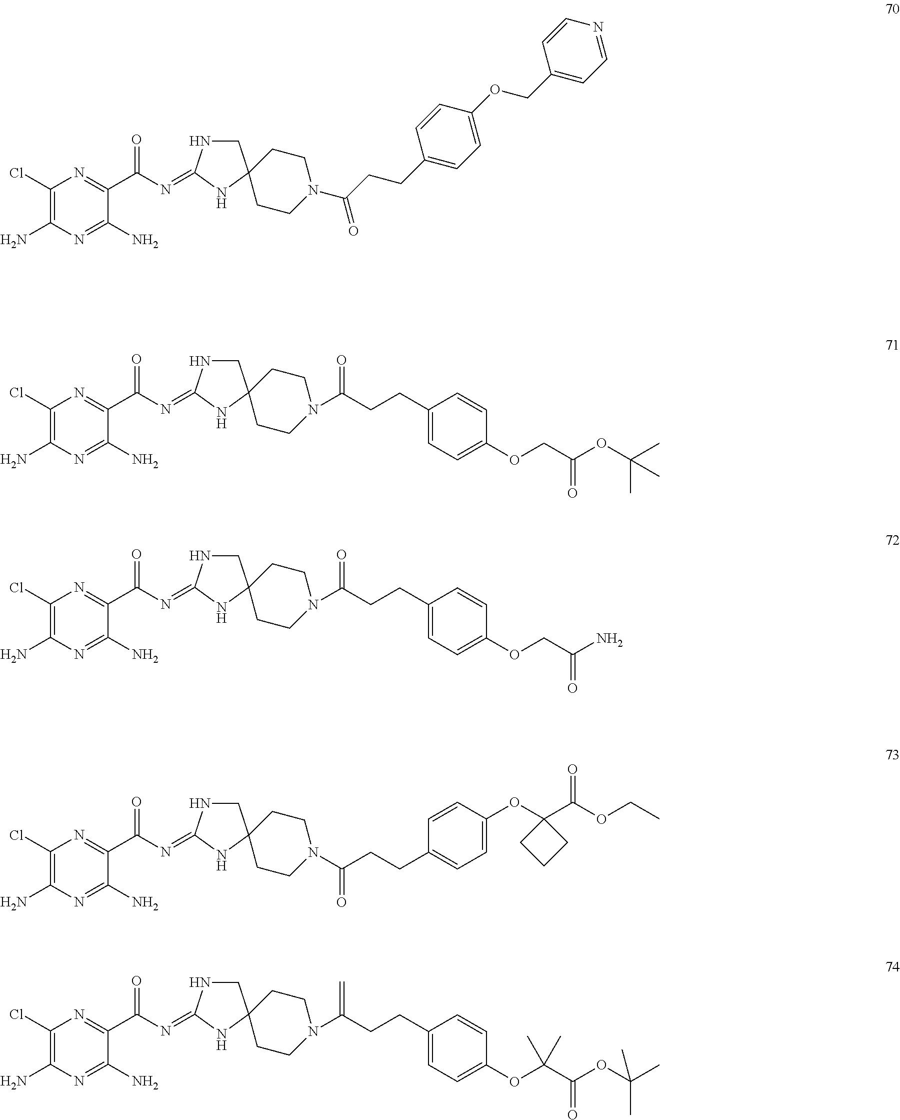 [SMILES: Nc1nc(N)c(C(=O)/N=C2\NCC3(CCN(C(=O)CCc4ccc(OCc5ccncc5)cc4)CC3)N2)nc1Cl ; CC(C)(C)OC(=O)COc1ccc(CCC(=O)N2CCC3(CC2)CN/C(=N\C(=O)c2nc(Cl)c(N)nc2N)N3)cc1 ; NC(=O)COc1ccc(CCC(=O)N2CCC3(CC2)CN/C(=N\C(=O)c2nc(Cl)c(N)nc2N)N3)cc1 ; CCOC(=O)C1(Oc2ccc(CCC(=O)N3CCC4(CC3)CN/C(=N\C(=O)c3nc(Cl)c(N)nc3N)N4)cc2)CCC1 ; C=C(CCc1ccc(OC(C)(C)C(=O)OC(C)(C)C)cc1)N1CCC2(CC1)CN/C(=N\C(=O)c1nc(Cl)c(N)nc1N)N2]

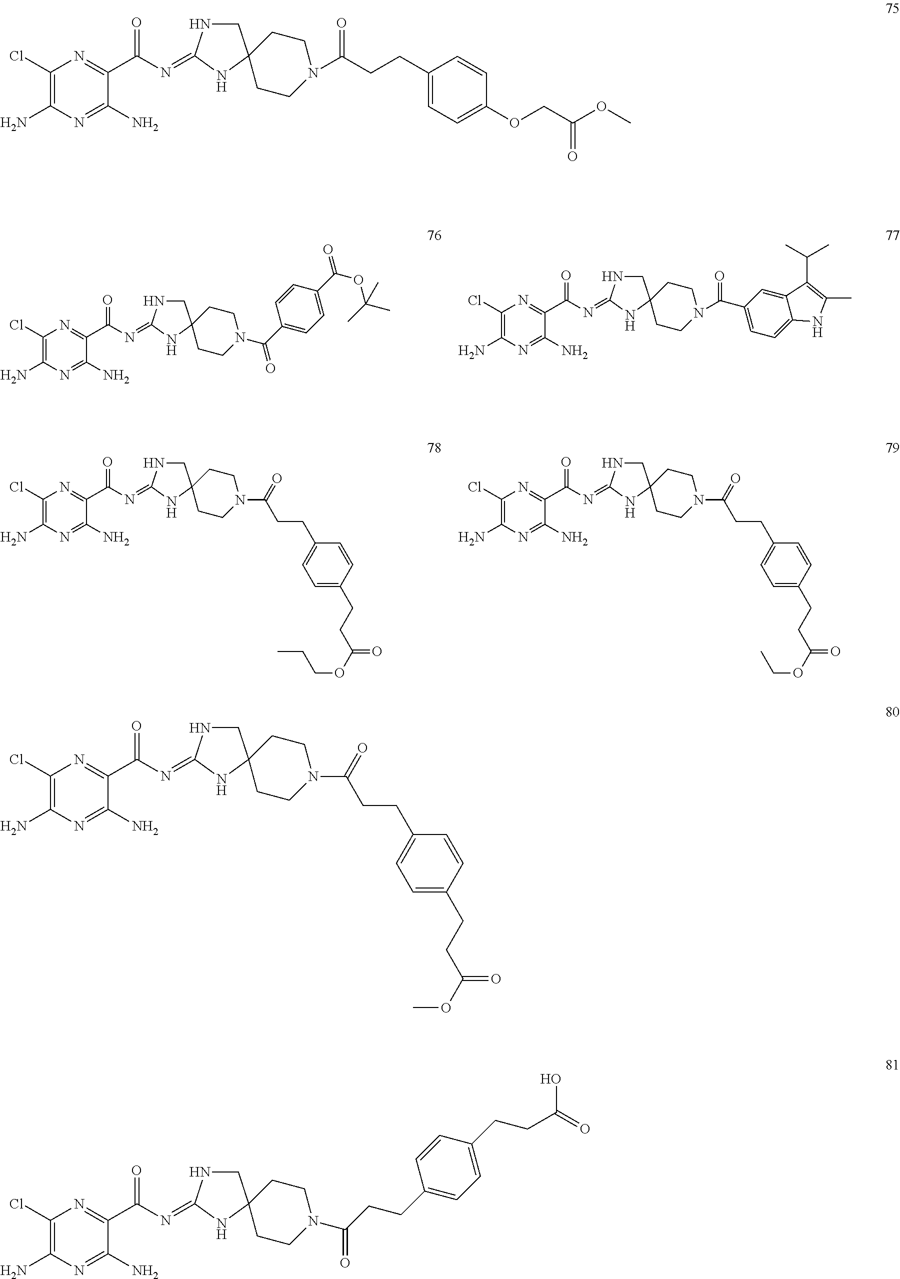

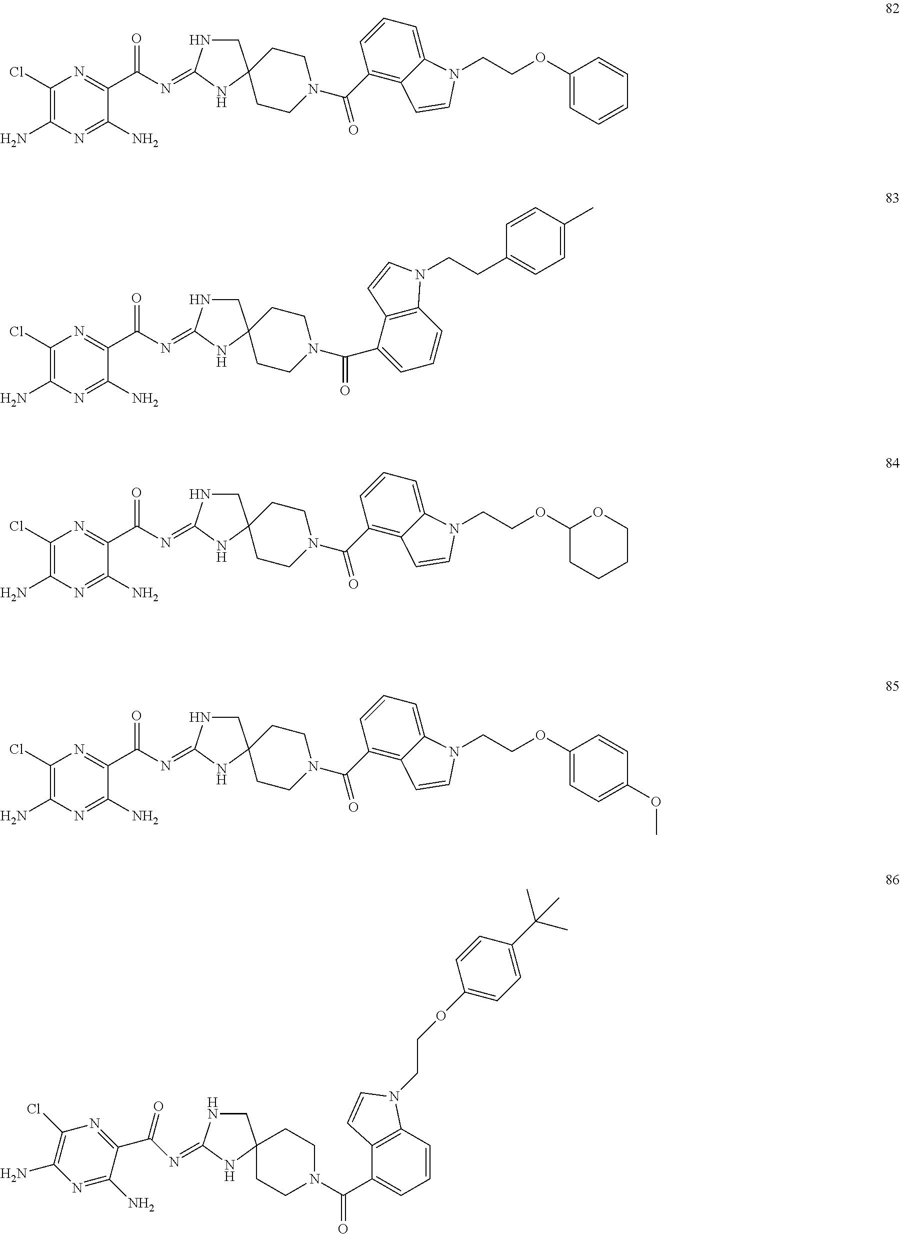 [SMILES: Nc1nc(N)c(C(=O)/N=C2\NCC3(CCN(C(=O)c4cccc5c4ccn5CCOc4ccccc4)CC3)N2)nc1Cl ; Cc1ccc(CCn2ccc3c(C(=O)N4CCC5(CC4)CN/C(=N\C(=O)c4nc(Cl)c(N)nc4N)N5)cccc32)cc1 ; Nc1nc(N)c(C(=O)/N=C2\NCC3(CCN(C(=O)c4cccc5c4ccn5CCOC4CCCCO4)CC3)N2)nc1Cl ; COc1ccc(OCCn2ccc3c(C(=O)N4CCC5(CC4)CN/C(=N\C(=O)c4nc(Cl)c(N)nc4N)N5)cccc32)cc1 ; CC(C)(C)c1ccc(OCCn2ccc3c(C(=O)N4CCC5(CC4)CN/C(=N\C(=O)c4nc(Cl)c(N)nc4N)N5)cccc32)cc1]

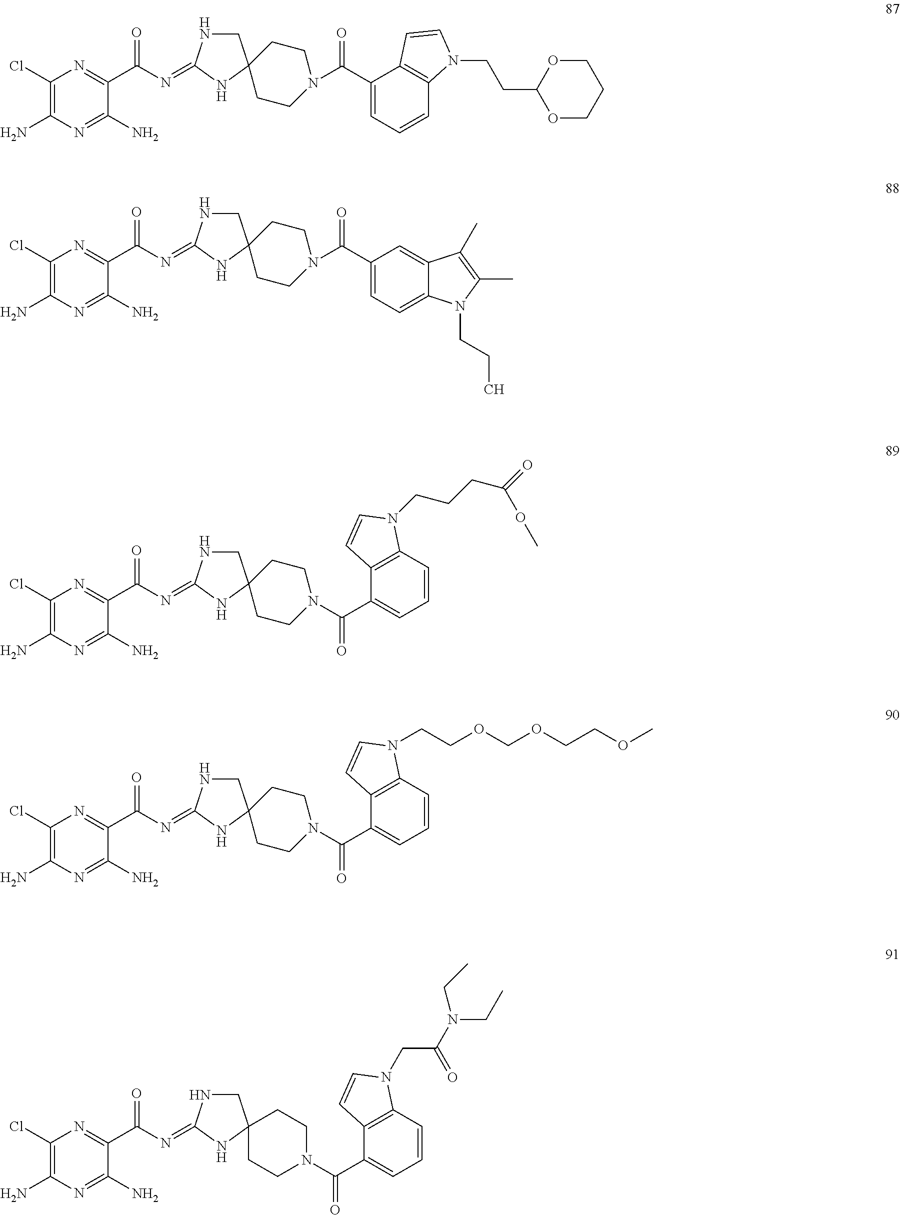 [SMILES: Nc1nc(N)c(C(=O)/N=C2\NCC3(CCN(C(=O)c4cccc5c4ccn5CCC4OCCCO4)CC3)N2)nc1Cl ; CCCn1c(C)c(C)c2cc(C(=O)N3CCC4(CC3)CN/C(=N\C(=O)c3nc(Cl)c(N)nc3N)N4)ccc21 ; COC(=O)CCCn1ccc2c(C(=O)N3CCC4(CC3)CN/C(=N\C(=O)c3nc(Cl)c(N)nc3N)N4)cccc21 ; COCCOCOCCn1ccc2c(C(=O)N3CCC4(CC3)CN/C(=N\C(=O)c3nc(Cl)c(N)nc3N)N4)cccc21 ; CCN(CC)C(=O)Cn1ccc2c(C(=O)N3CCC4(CC3)CN/C(=N\C(=O)c3nc(Cl)c(N)nc3N)N4)cccc21]

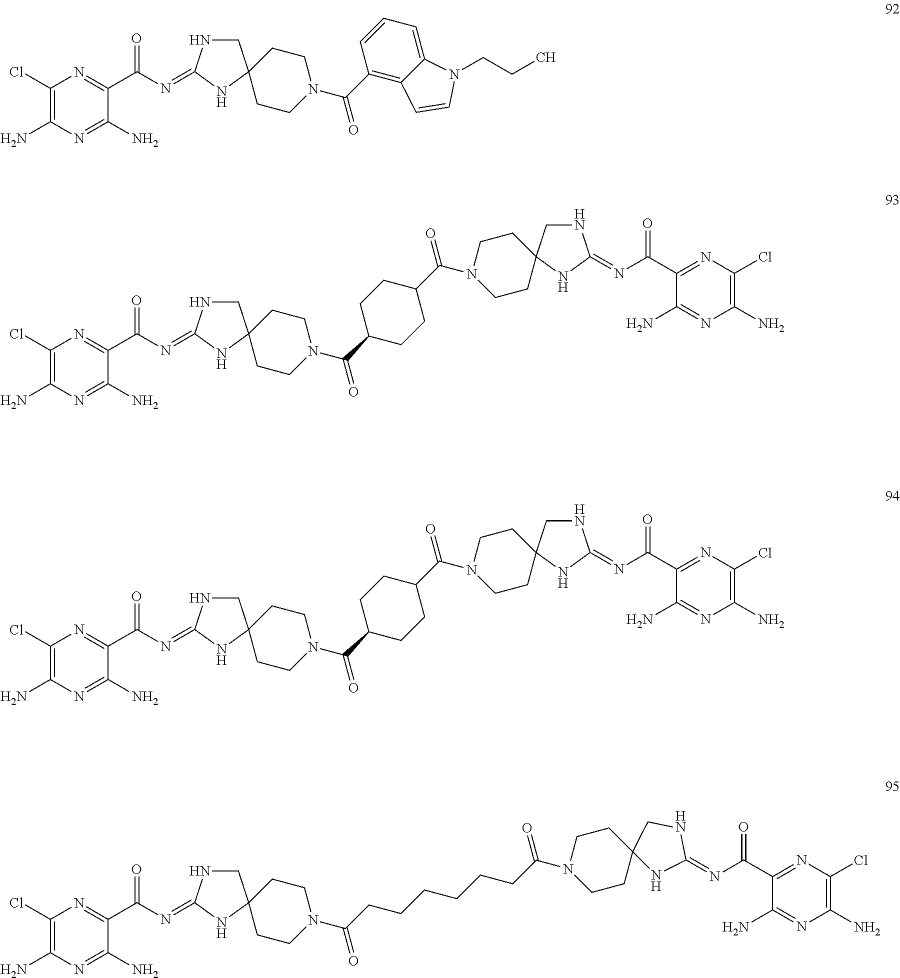

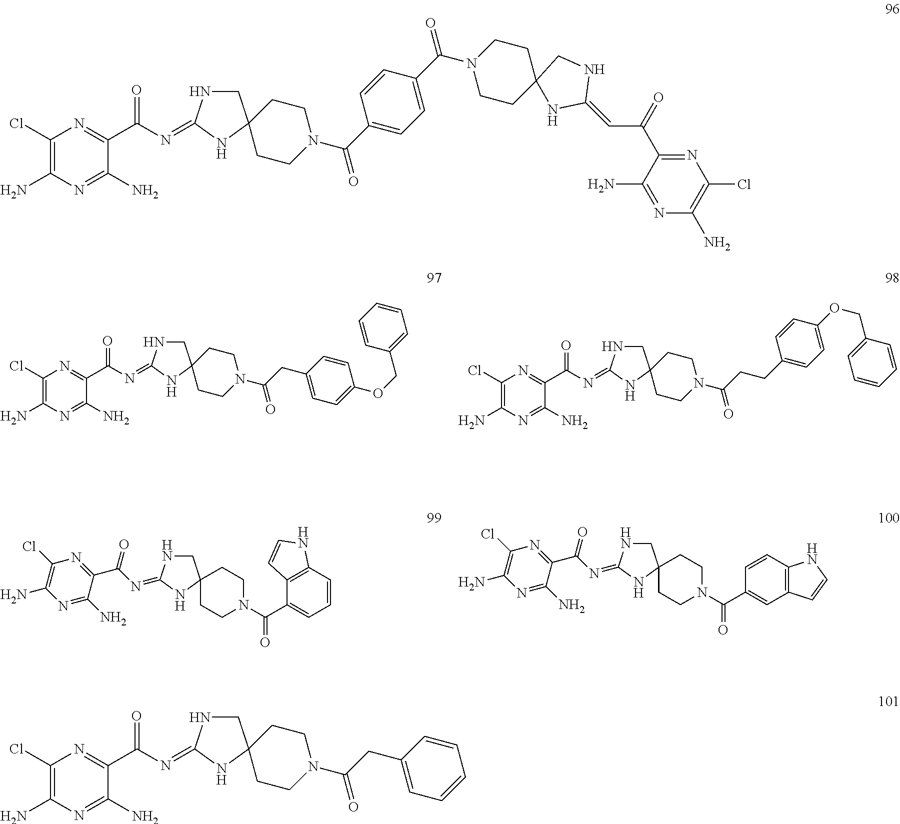

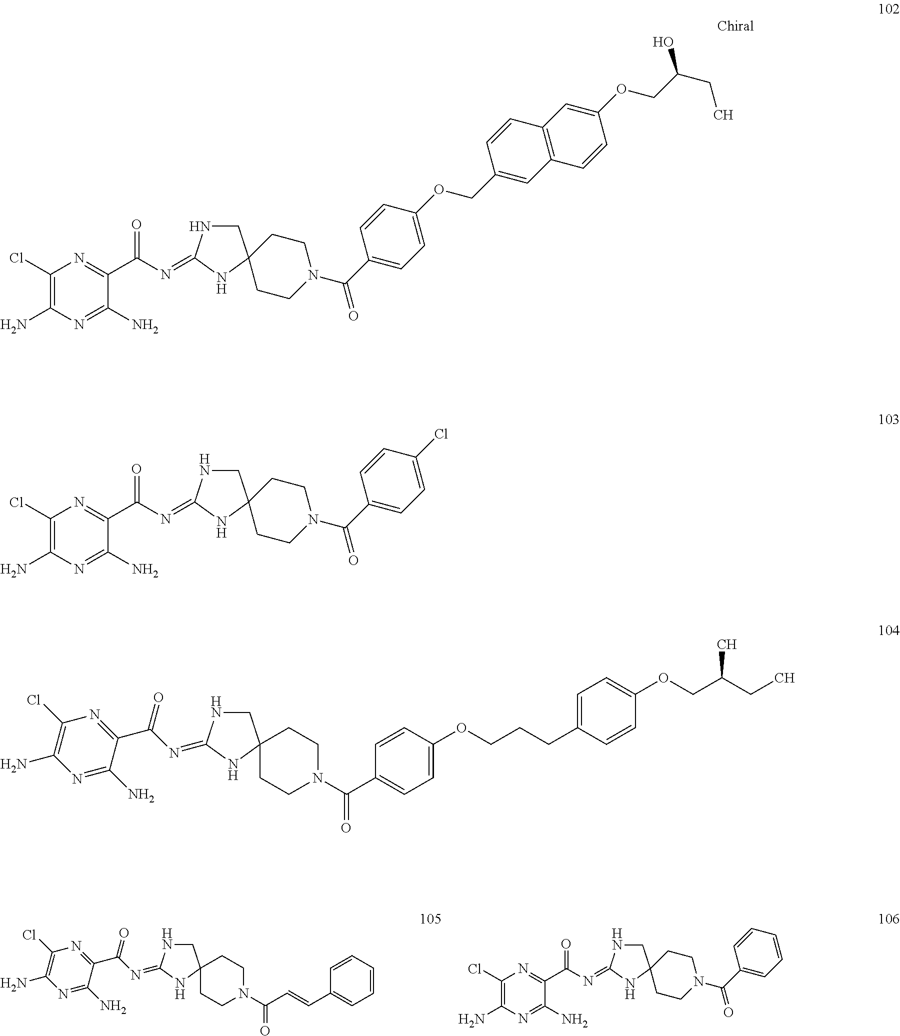

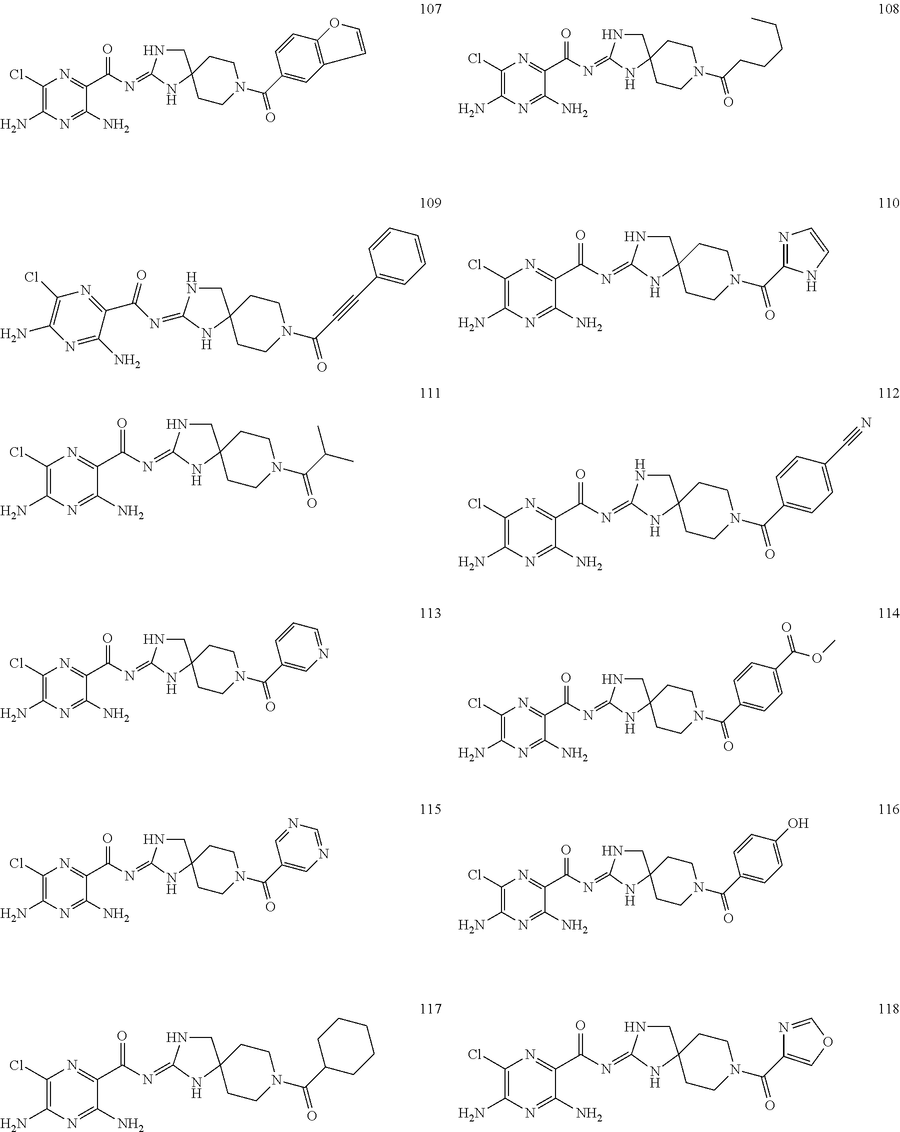

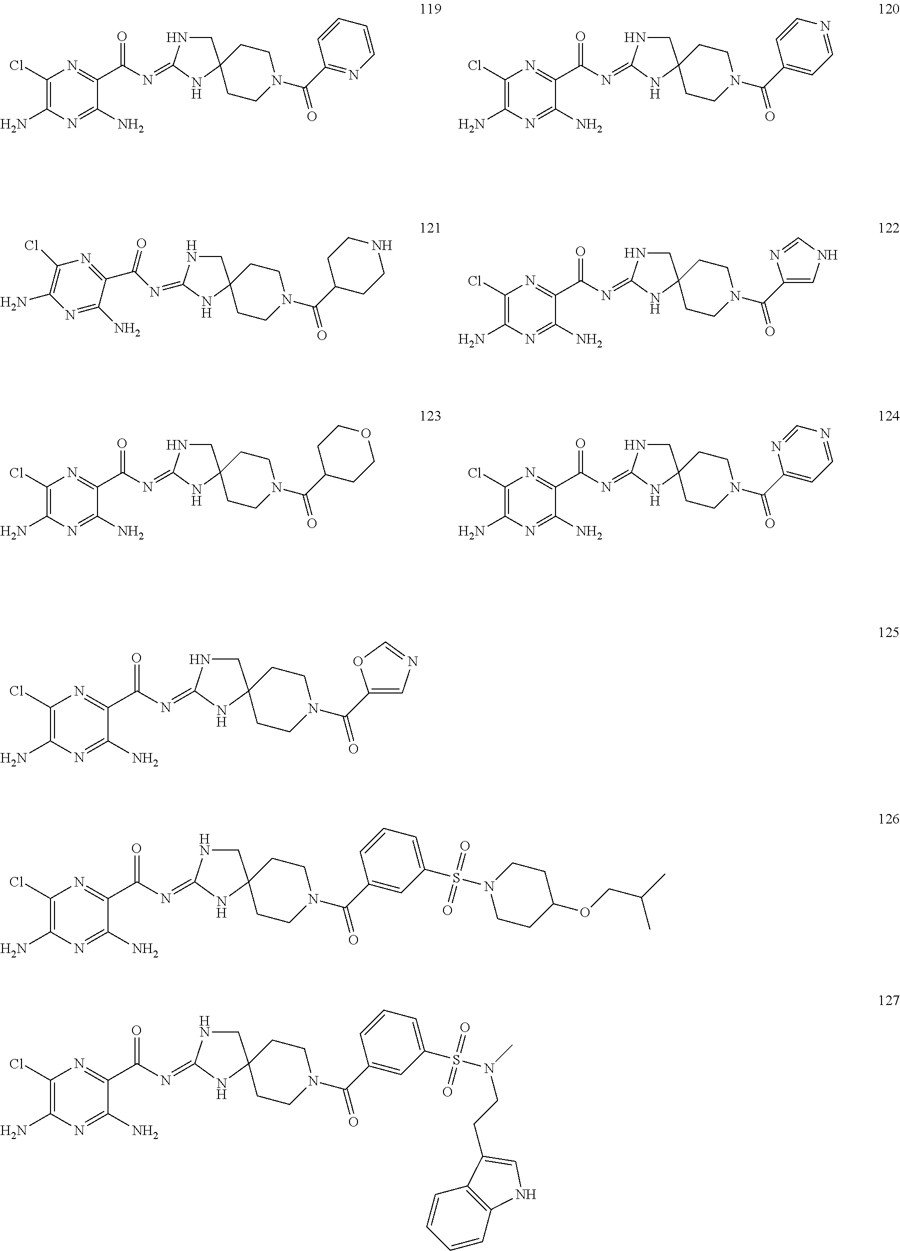

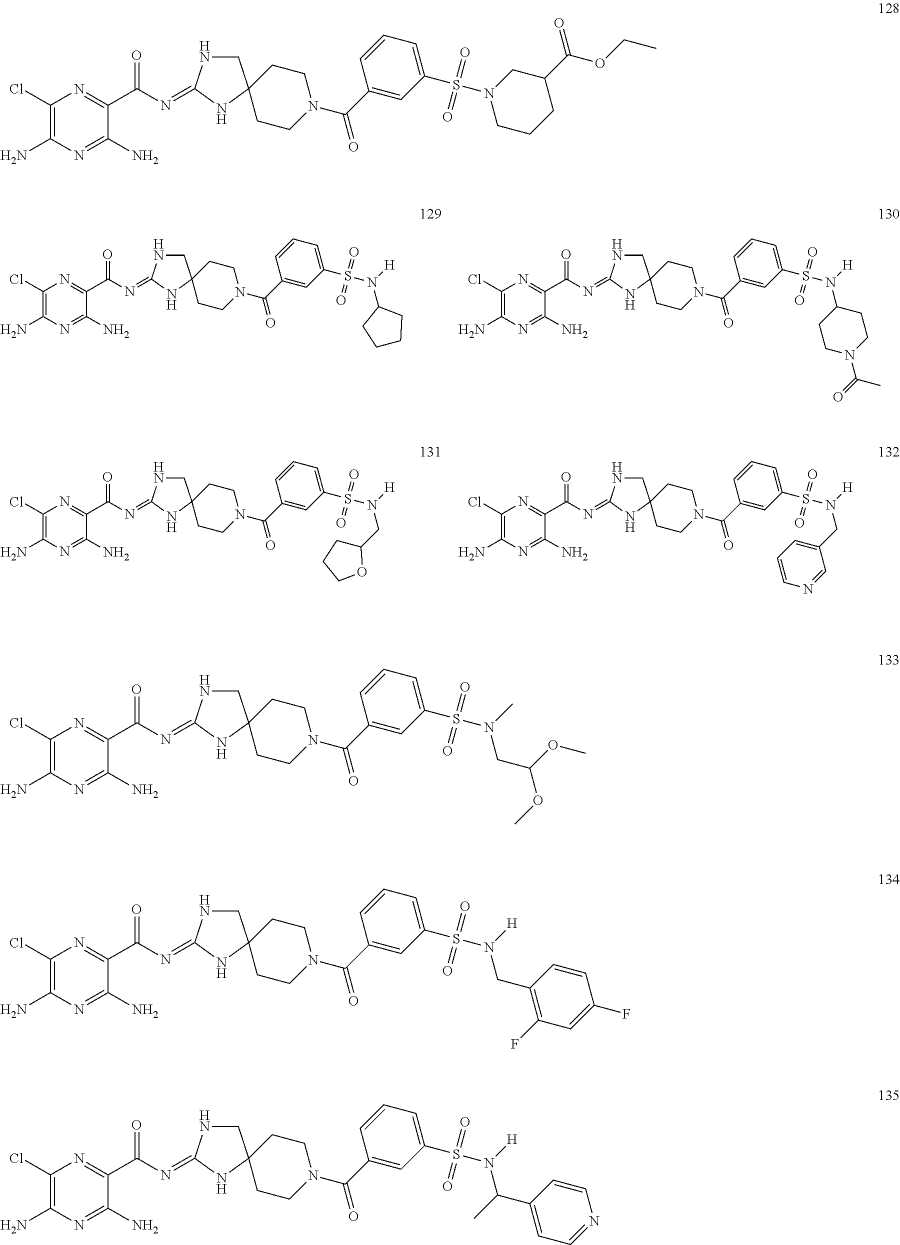

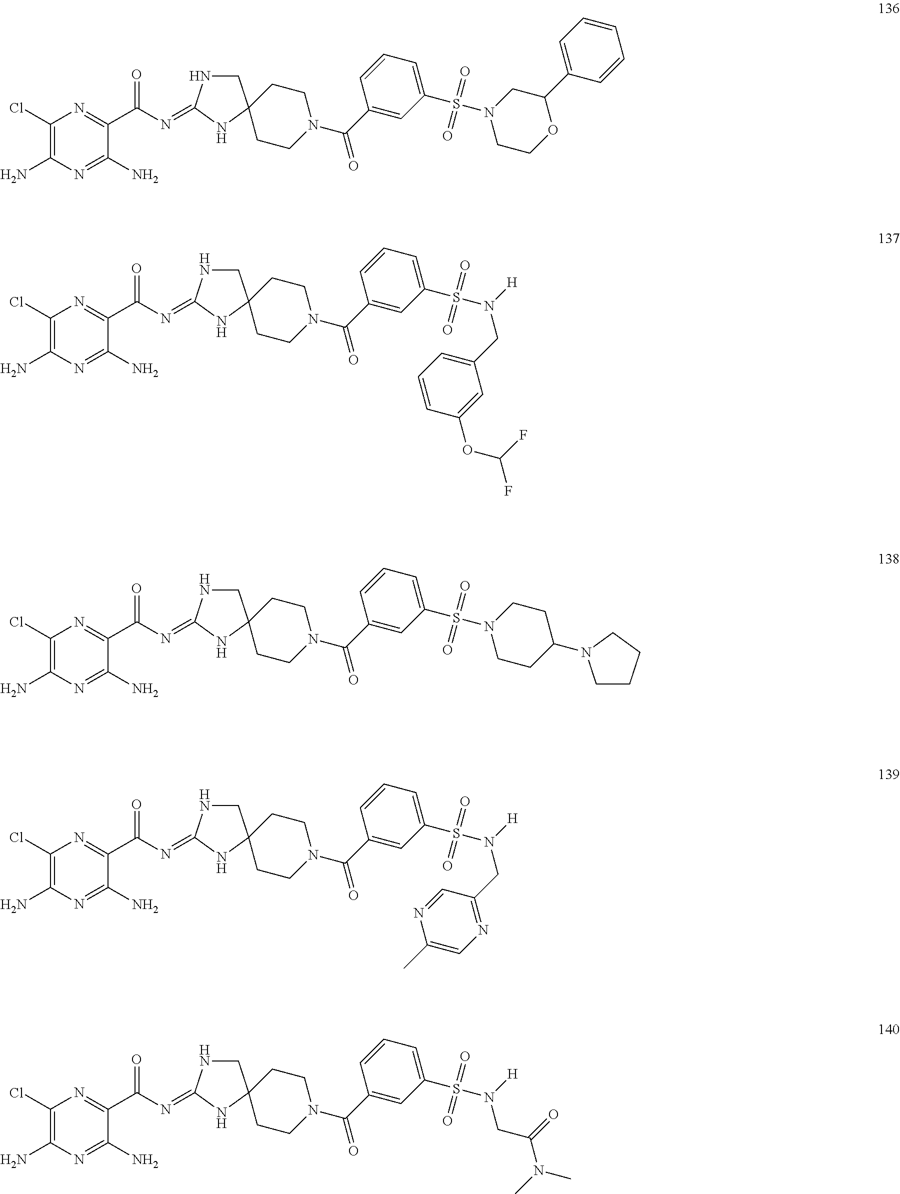 [SMILES: Nc1nc(N)c(C(=O)/N=C2\NCC3(CCN(C(=O)c4cccc(S(=O)(=O)N5CCOC(c6ccccc6)C5)c4)CC3)N2)nc1Cl ; Nc1nc(N)c(C(=O)/N=C2\NCC3(CCN(C(=O)c4cccc(S(=O)(=O)NCc5cccc(OC(F)F)c5)c4)CC3)N2)nc1Cl ; Nc1nc(N)c(C(=O)/N=C2\NCC3(CCN(C(=O)c4cccc(S(=O)(=O)N5CCC(N6CCCC6)CC5)c4)CC3)N2)nc1Cl ; Cc1cnc(CNS(=O)(=O)c2cccc(C(=O)N3CCC4(CC3)CN/C(=N\C(=O)c3nc(Cl)c(N)nc3N)N4)c2)cn1 ; CN(C)C(=O)CNS(=O)(=O)c1cccc(C(=O)N2CCC3(CC2)CN/C(=N\C(=O)c2nc(Cl)c(N)nc2N)N3)c1]

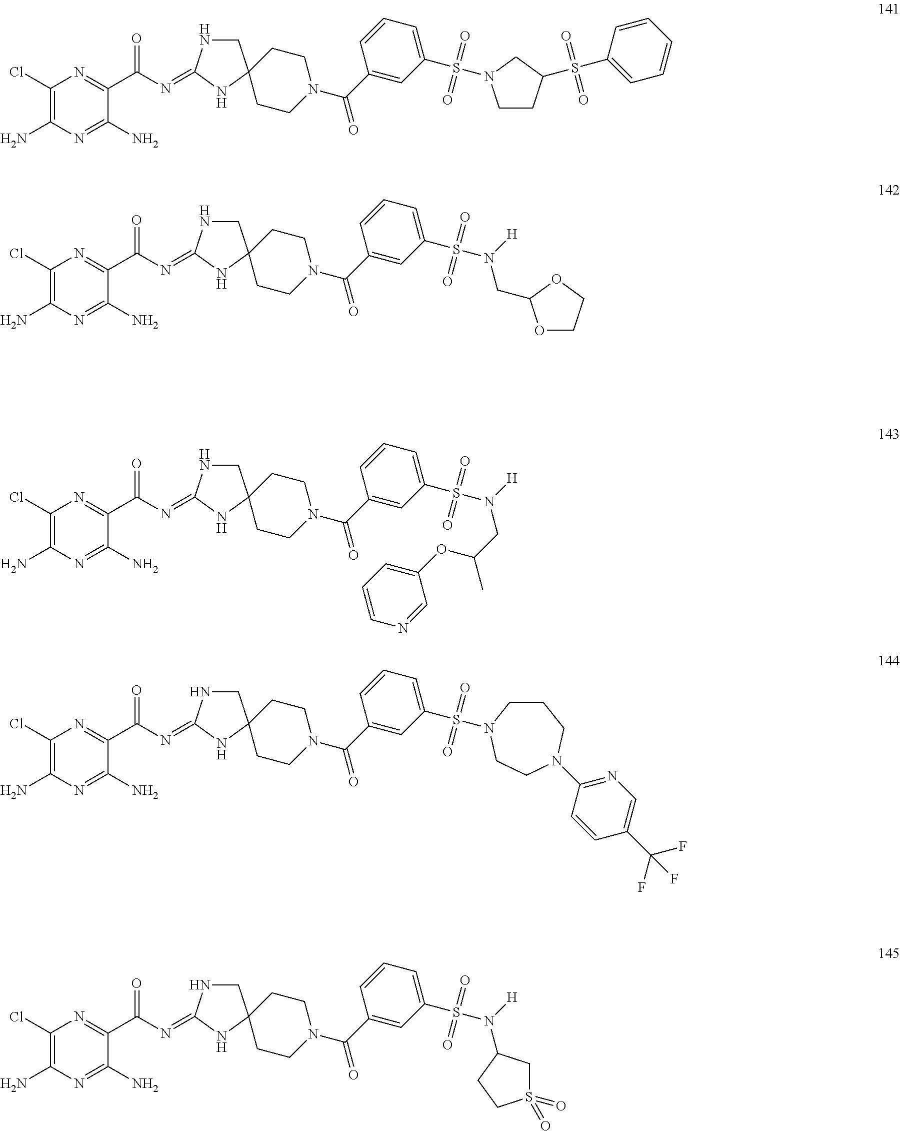

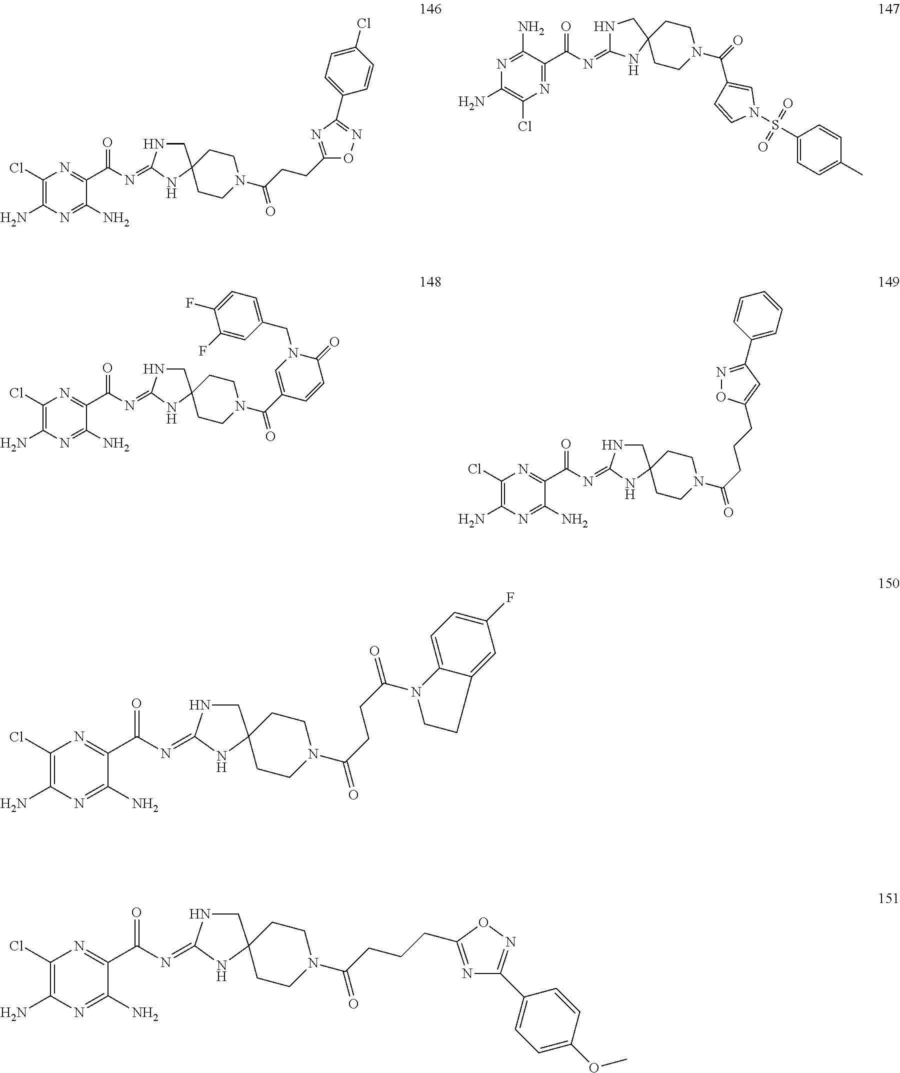 [SMILES: Nc1nc(N)c(C(=O)/N=C2\NCC3(CCN(C(=O)CCc4nc(-c5ccc(Cl)cc5)no4)CC3)N2)nc1Cl ; Cc1ccc(S(=O)(=O)n2ccc(C(=O)N3CCC4(CC3)CN/C(=N\C(=O)c3nc(Cl)c(N)nc3N)N4)c2)cc1 ; Nc1nc(N)c(C(=O)/N=C2\NCC3(CCN(C(=O)c4ccc(=O)n(Cc5ccc(F)c(F)c5)c4)CC3)N2)nc1Cl ; Nc1nc(N)c(C(=O)/N=C2\NCC3(CCN(C(=O)CCCc4cc(-c5ccccc5)no4)CC3)N2)nc1Cl ; Nc1nc(N)c(C(=O)/N=C2\NCC3(CCN(C(=O)CCC(=O)N4CCc5cc(F)ccc54)CC3)N2)nc1Cl ; COc1ccc(-c2noc(CCCC(=O)N3CCC4(CC3)CN/C(=N\C(=O)c3nc(Cl)c(N)nc3N)N4)n2)cc1]

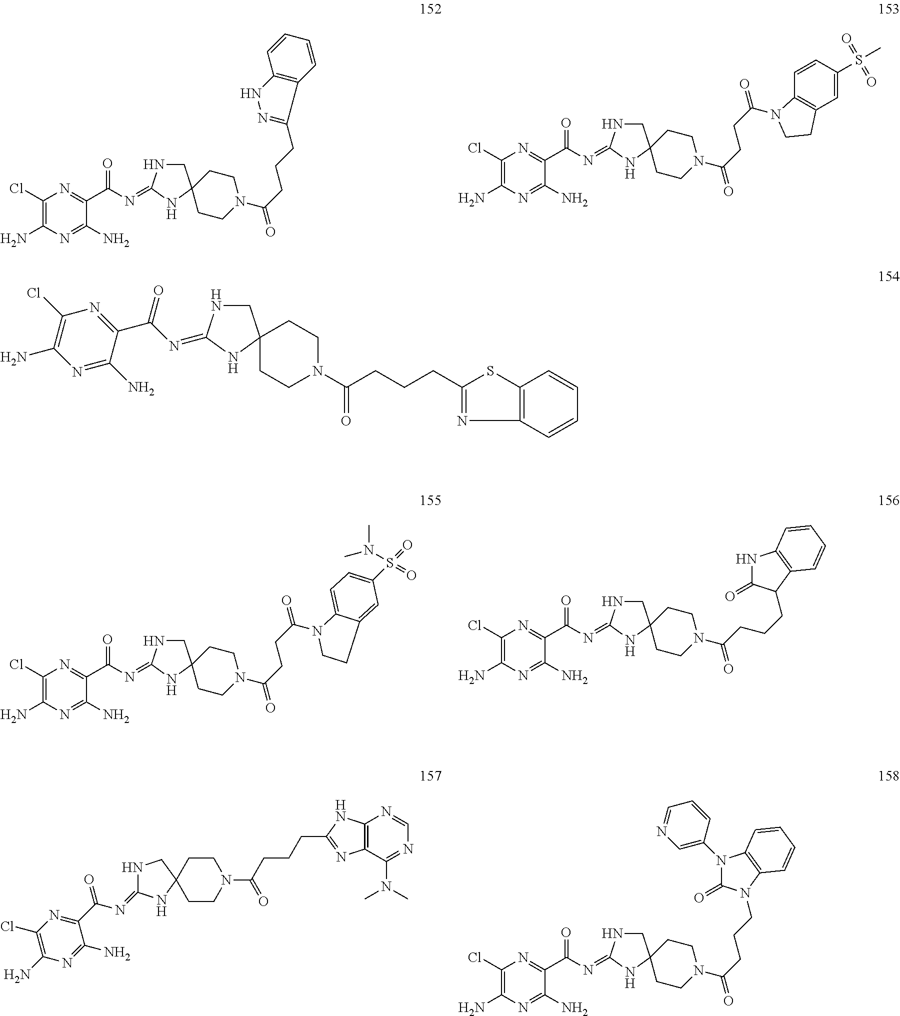

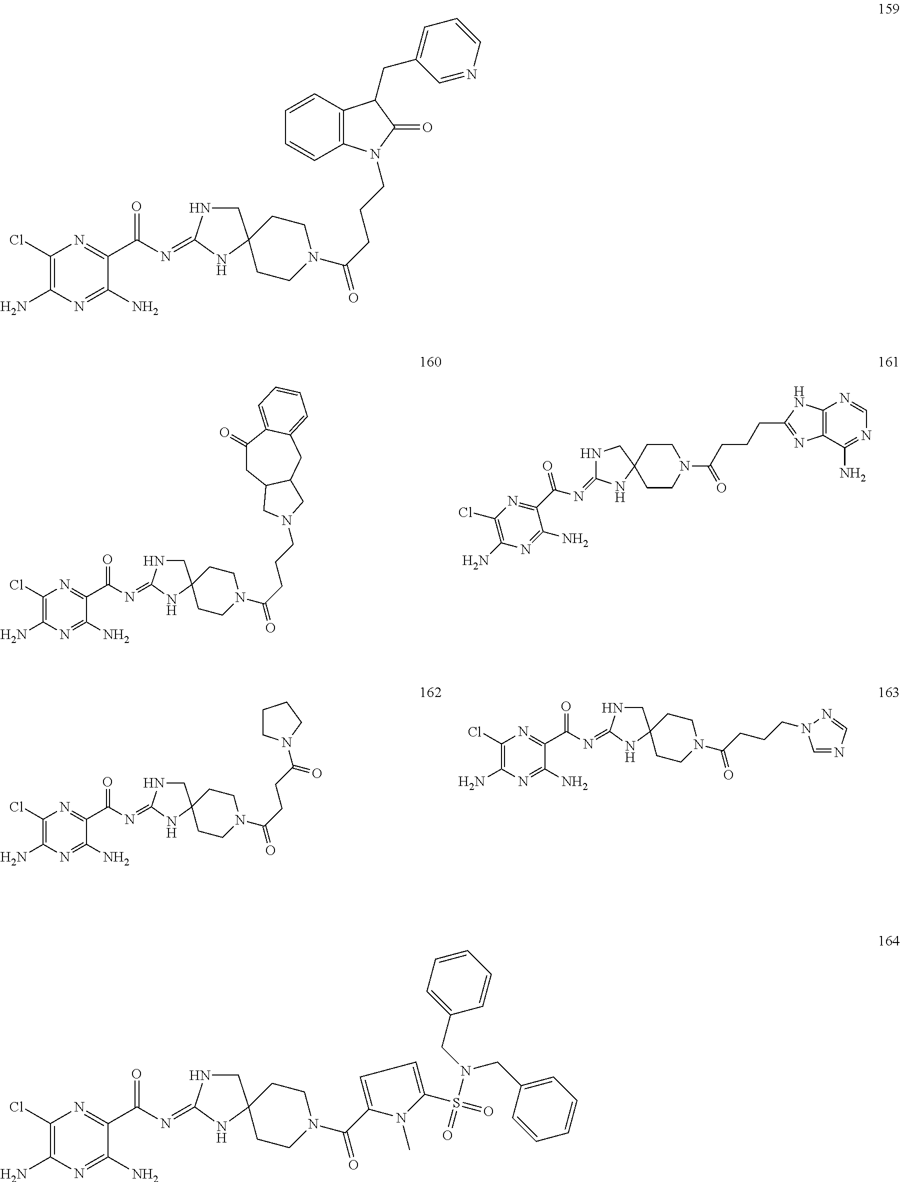 [SMILES: Nc1nc(N)c(C(=O)/N=C2\NCC3(CCN(C(=O)CCCN4C(=O)C(Cc5cccnc5)c5ccccc54)CC3)N2)nc1Cl ; Nc1nc(N)c(C(=O)/N=C2\NCC3(CCN(C(=O)CCCN4CC5CC(=O)c6ccccc6CC5C4)CC3)N2)nc1Cl ; Nc1nc(N)c(C(=O)/N=C2\NCC3(CCN(C(=O)CCCc4nc5c(N)ncnc5[nH]4)CC3)N2)nc1Cl ; Nc1nc(N)c(C(=O)/N=C2\NCC3(CCN(C(=O)CCC(=O)N4CCCC4)CC3)N2)nc1Cl ; Nc1nc(N)c(C(=O)/N=C2\NCC3(CCN(C(=O)CCCn4cncn4)CC3)N2)nc1Cl ; Cn1c(C(=O)N2CCC3(CC2)CN/C(=N\C(=O)c2nc(Cl)c(N)nc2N)N3)ccc1S(=O)(=O)N(Cc1ccccc1)Cc1ccccc1]

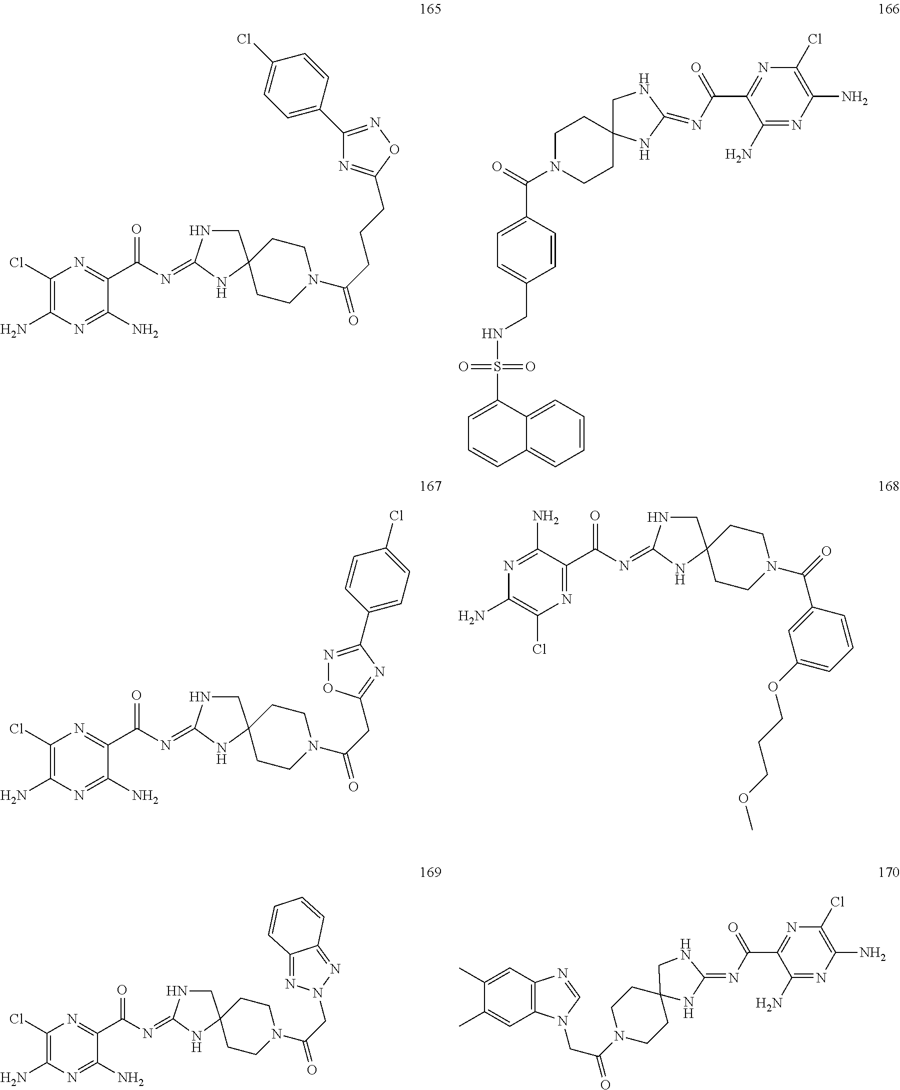 [SMILES: Nc1nc(N)c(C(=O)/N=C2\NCC3(CCN(C(=O)CCCc4nc(-c5ccc(Cl)cc5)no4)CC3)N2)nc1Cl ; Nc1nc(N)c(C(=O)/N=C2\NCC3(CCN(C(=O)c4ccc(CNS(=O)(=O)c5cccc6ccccc56)cc4)CC3)N2)nc1Cl ; Nc1nc(N)c(C(=O)/N=C2\NCC3(CCN(C(=O)Cc4nc(-c5ccc(Cl)cc5)no4)CC3)N2)nc1Cl ; COCCCOc1cccc(C(=O)N2CCC3(CC2)CN/C(=N\C(=O)c2nc(Cl)c(N)nc2N)N3)c1 ; Nc1nc(N)c(C(=O)/N=C2\NCC3(CCN(C(=O)Cn4nc5ccccc5n4)CC3)N2)nc1Cl ; Cc1cc2ncn(CC(=O)N3CCC4(CC3)CN/C(=N\C(=O)c3nc(Cl)c(N)nc3N)N4)c2cc1C]

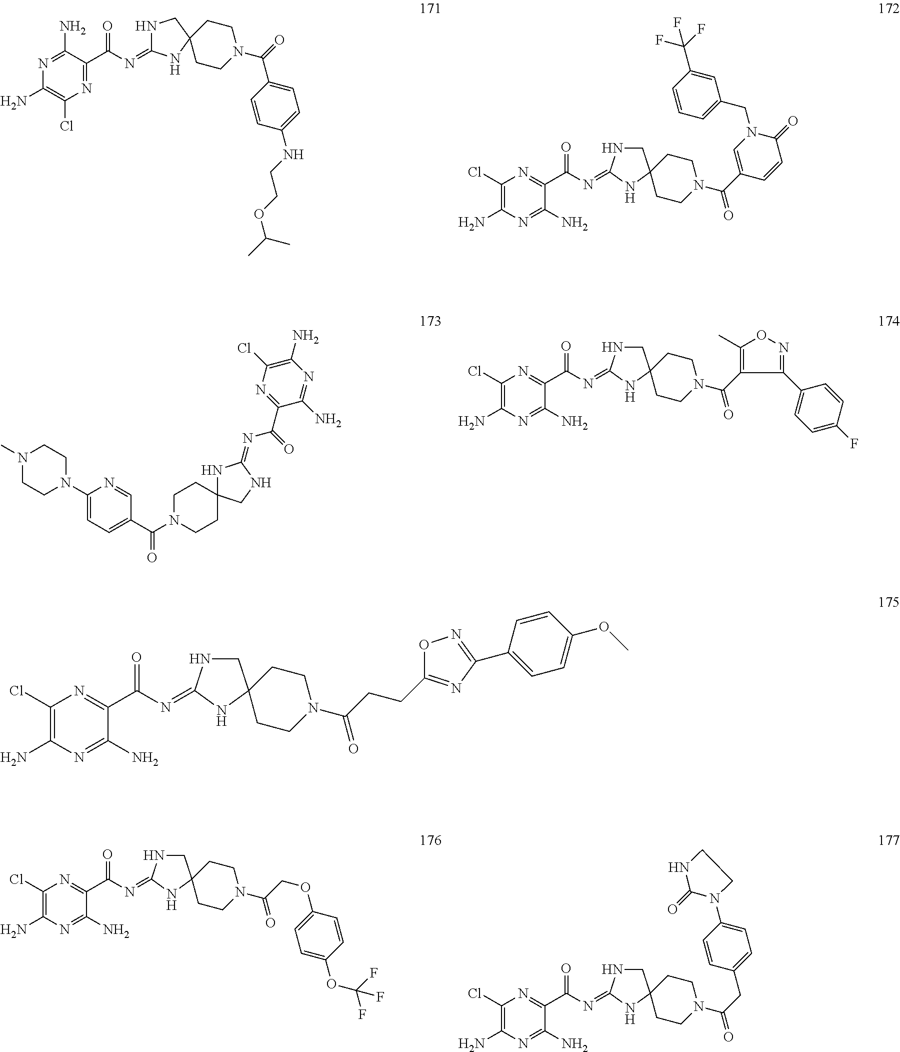

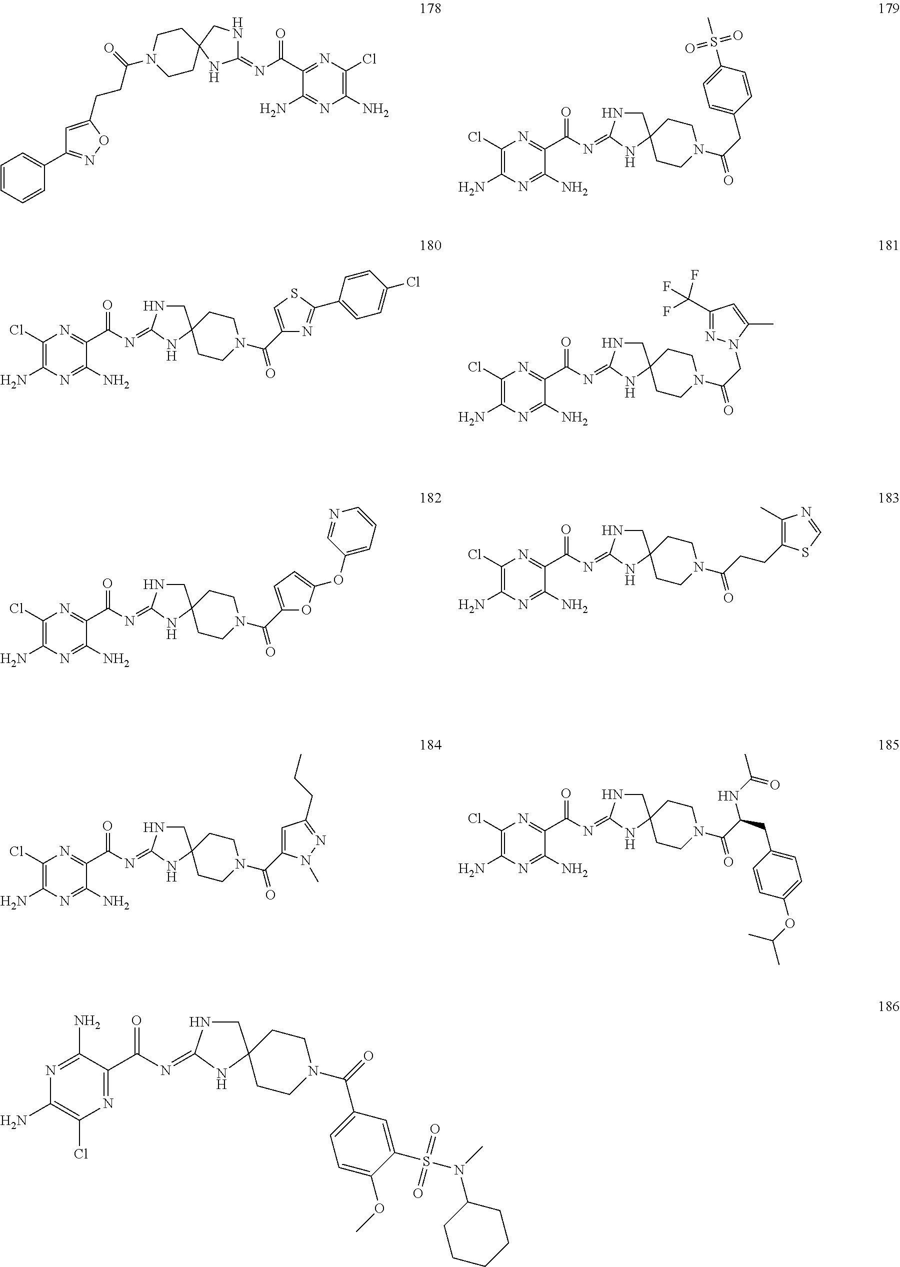

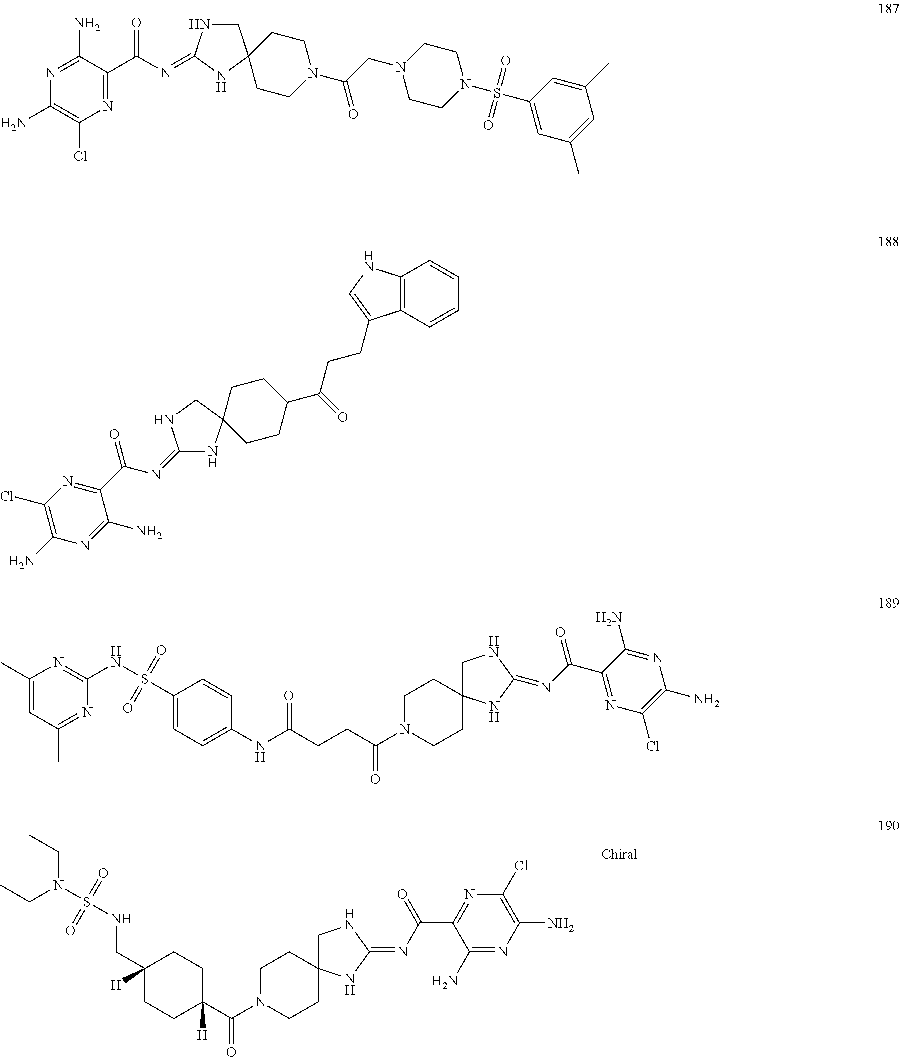

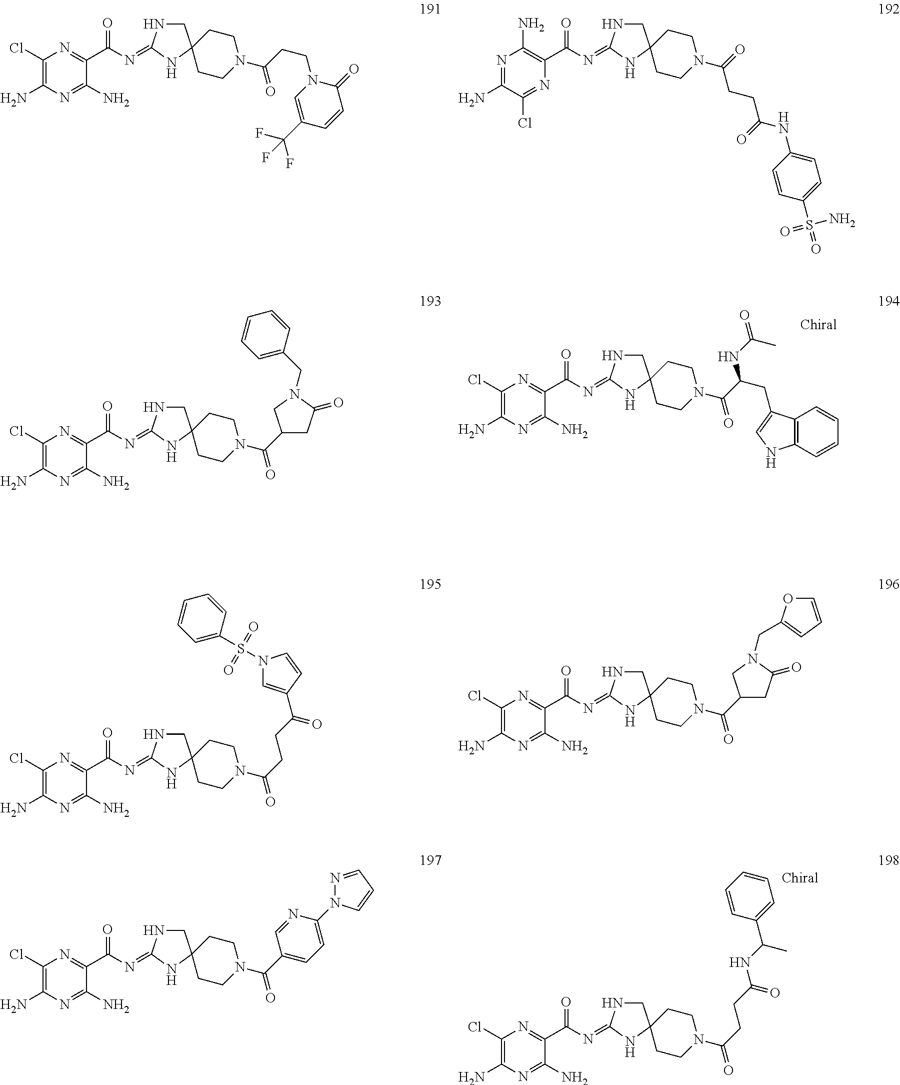

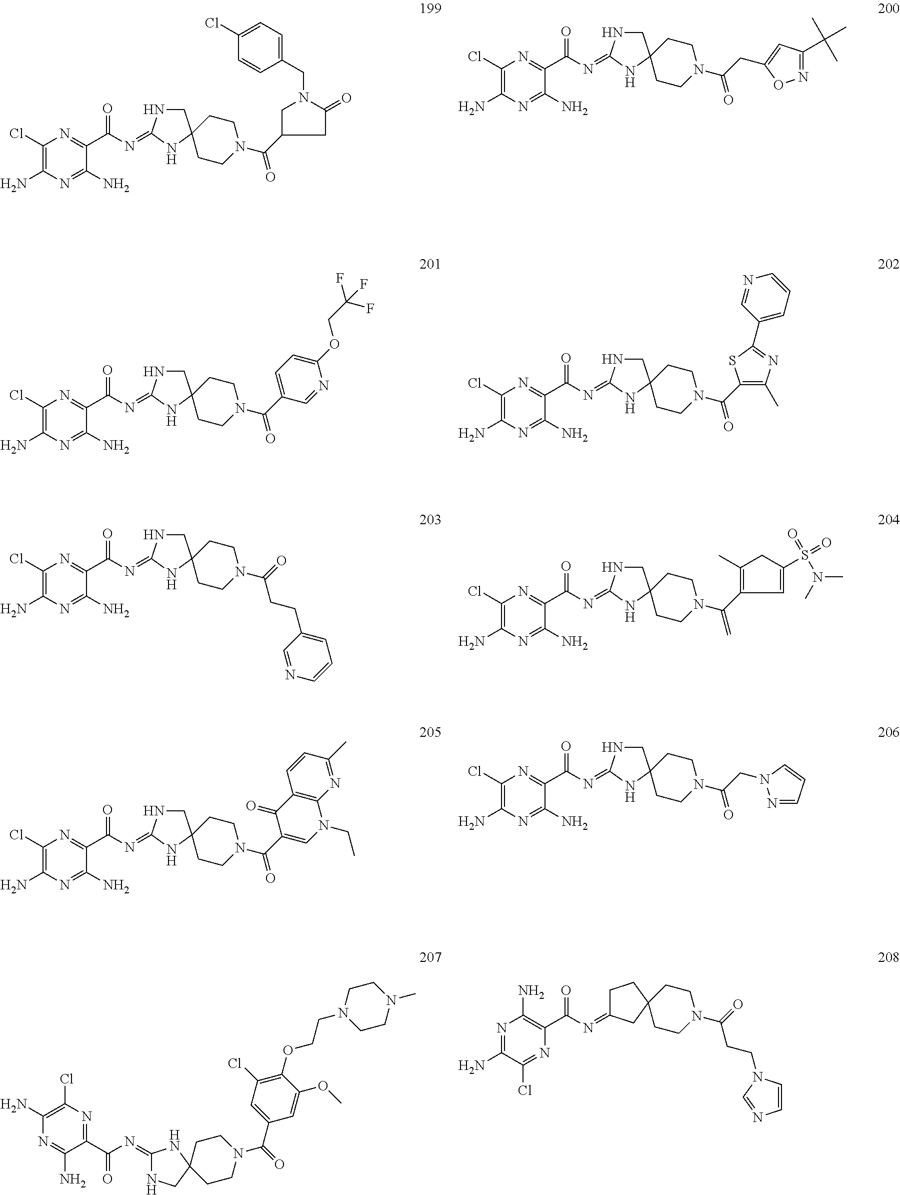

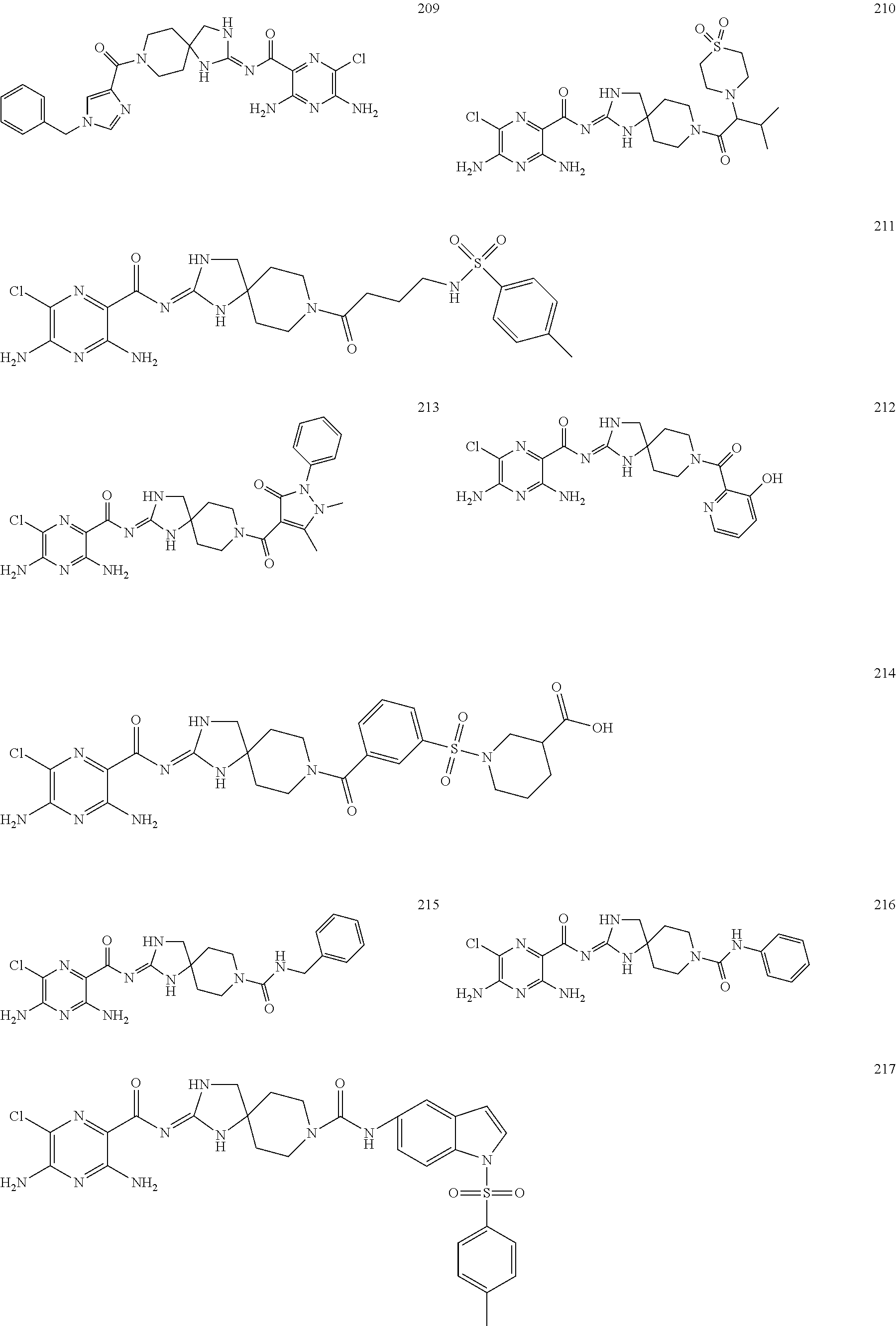

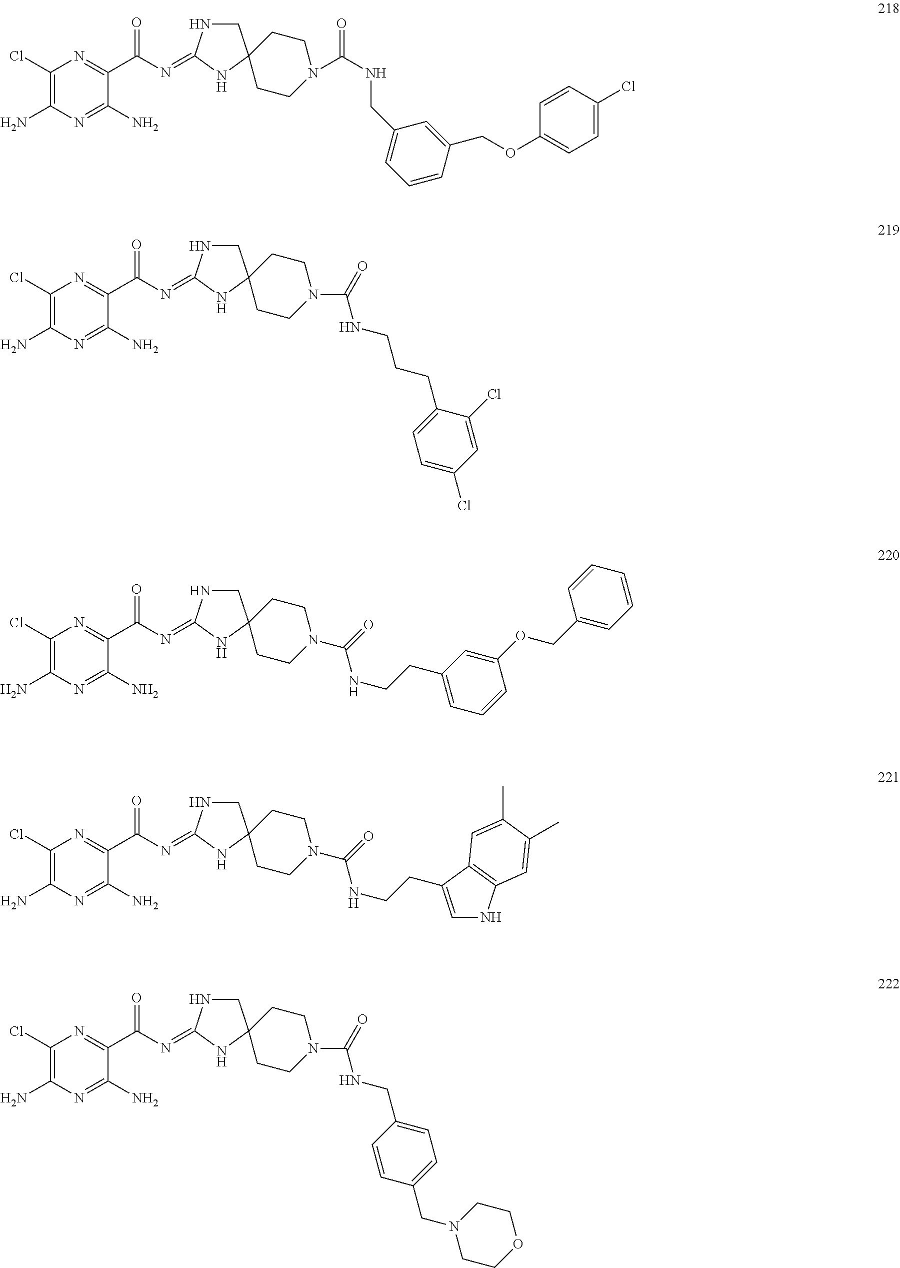 [SMILES: Nc1nc(N)c(C(=O)/N=C2\NCC3(CCN(C(=O)NCc4cccc(COc5ccc(Cl)cc5)c4)CC3)N2)nc1Cl ; Nc1nc(N)c(C(=O)/N=C2\NCC3(CCN(C(=O)NCCCc4ccc(Cl)cc4Cl)CC3)N2)nc1Cl ; Nc1nc(N)c(C(=O)/N=C2\NCC3(CCN(C(=O)NCCc4cccc(OCc5ccccc5)c4)CC3)N2)nc1Cl ; Cc1cc2[nH]cc(CCNC(=O)N3CCC4(CC3)CN/C(=N\C(=O)c3nc(Cl)c(N)nc3N)N4)c2cc1C ; Nc1nc(N)c(C(=O)/N=C2\NCC3(CCN(C(=O)NCc4ccc(CN5CCOCC5)cc4)CC3)N2)nc1Cl]

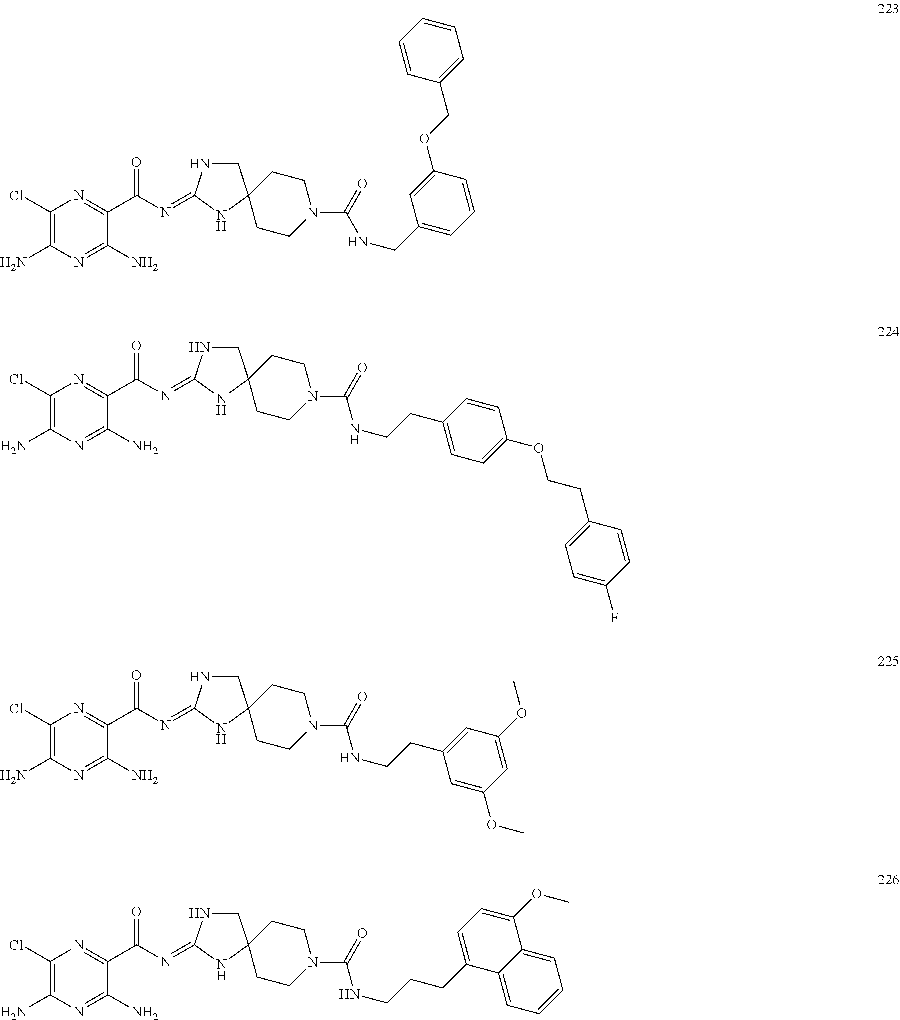 [SMILES: Nc1nc(N)c(C(=O)/N=C2\NCC3(CCN(C(=O)NCc4cccc(OCc5ccccc5)c4)CC3)N2)nc1Cl ; Nc1nc(N)c(C(=O)/N=C2\NCC3(CCN(C(=O)NCCc4ccc(OCCc5ccc(F)cc5)cc4)CC3)N2)nc1Cl ; COc1cc(CCNC(=O)N2CCC3(CC2)CN/C(=N\C(=O)c2nc(Cl)c(N)nc2N)N3)cc(OC)c1 ; COc1ccc(CCCNC(=O)N2CCC3(CC2)CN/C(=N\C(=O)c2nc(Cl)c(N)nc2N)N3)c2ccccc12]

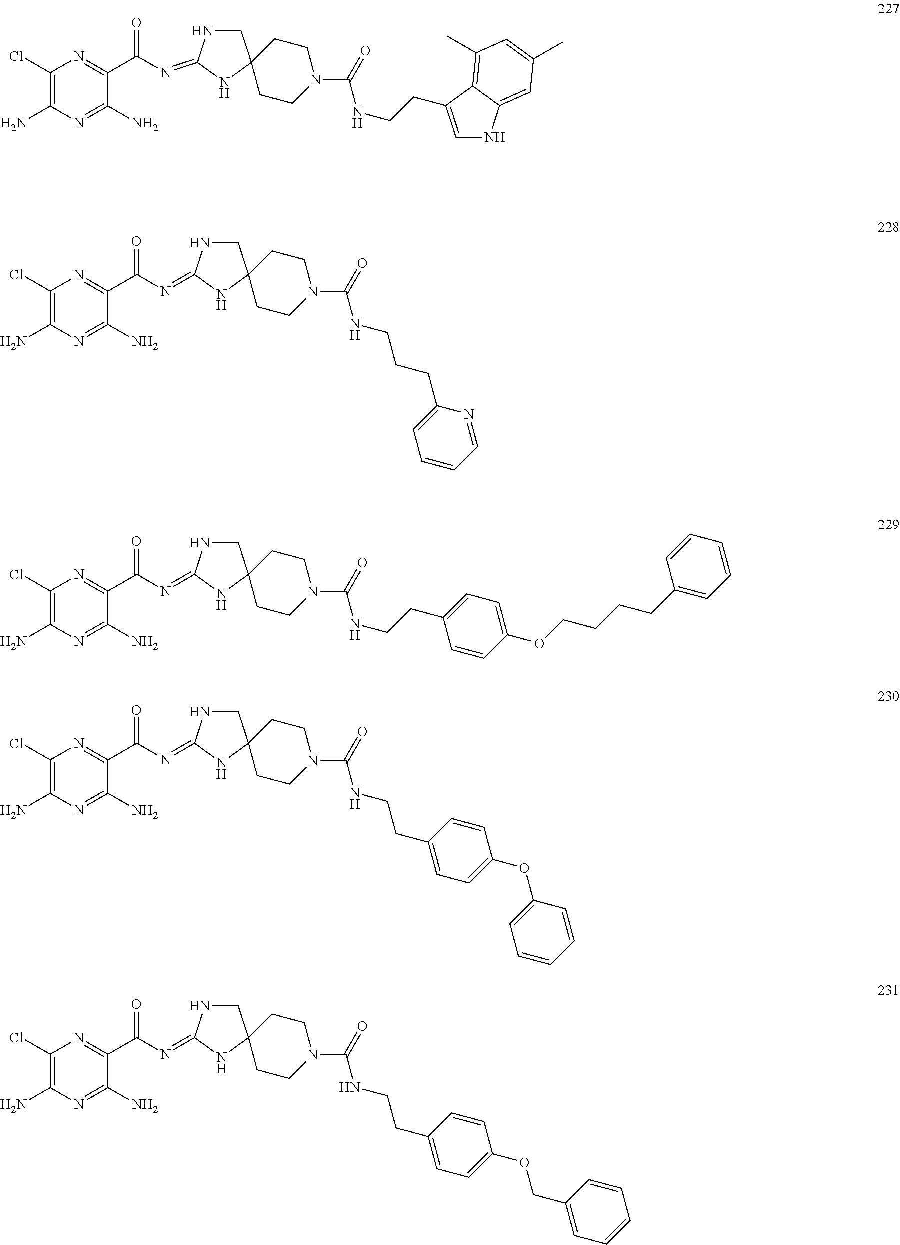 [SMILES: Cc1cc(C)c2c(CCNC(=O)N3CCC4(CC3)CN/C(=N\C(=O)c3nc(Cl)c(N)nc3N)N4)c[nH]c2c1 ; Nc1nc(N)c(C(=O)/N=C2\NCC3(CCN(C(=O)NCCCc4ccccn4)CC3)N2)nc1Cl ; Nc1nc(N)c(C(=O)/N=C2\NCC3(CCN(C(=O)NCCc4ccc(OCCCCc5ccccc5)cc4)CC3)N2)nc1Cl ; Nc1nc(N)c(C(=O)/N=C2\NCC3(CCN(C(=O)NCCc4ccc(Oc5ccccc5)cc4)CC3)N2)nc1Cl ; Nc1nc(N)c(C(=O)/N=C2\NCC3(CCN(C(=O)NCCc4ccc(OCc5ccccc5)cc4)CC3)N2)nc1Cl]

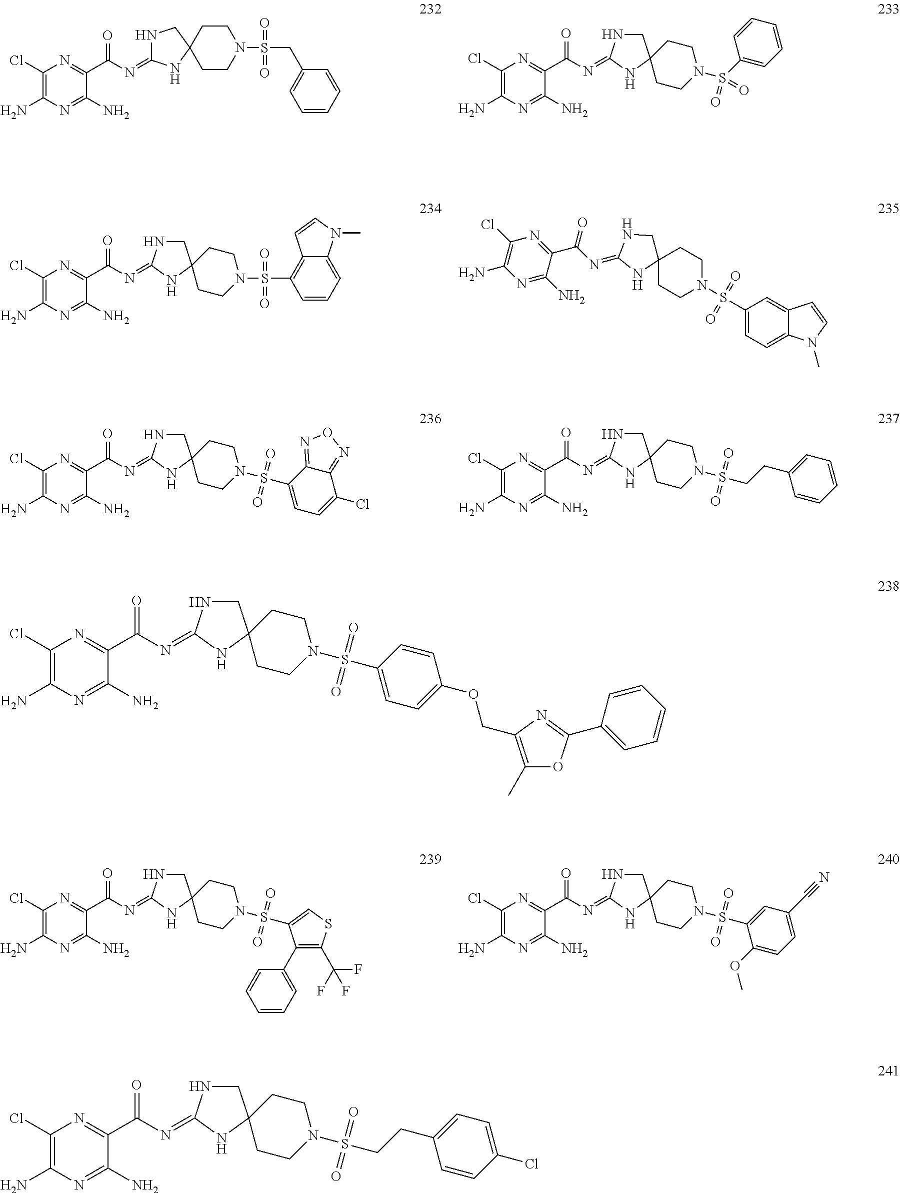

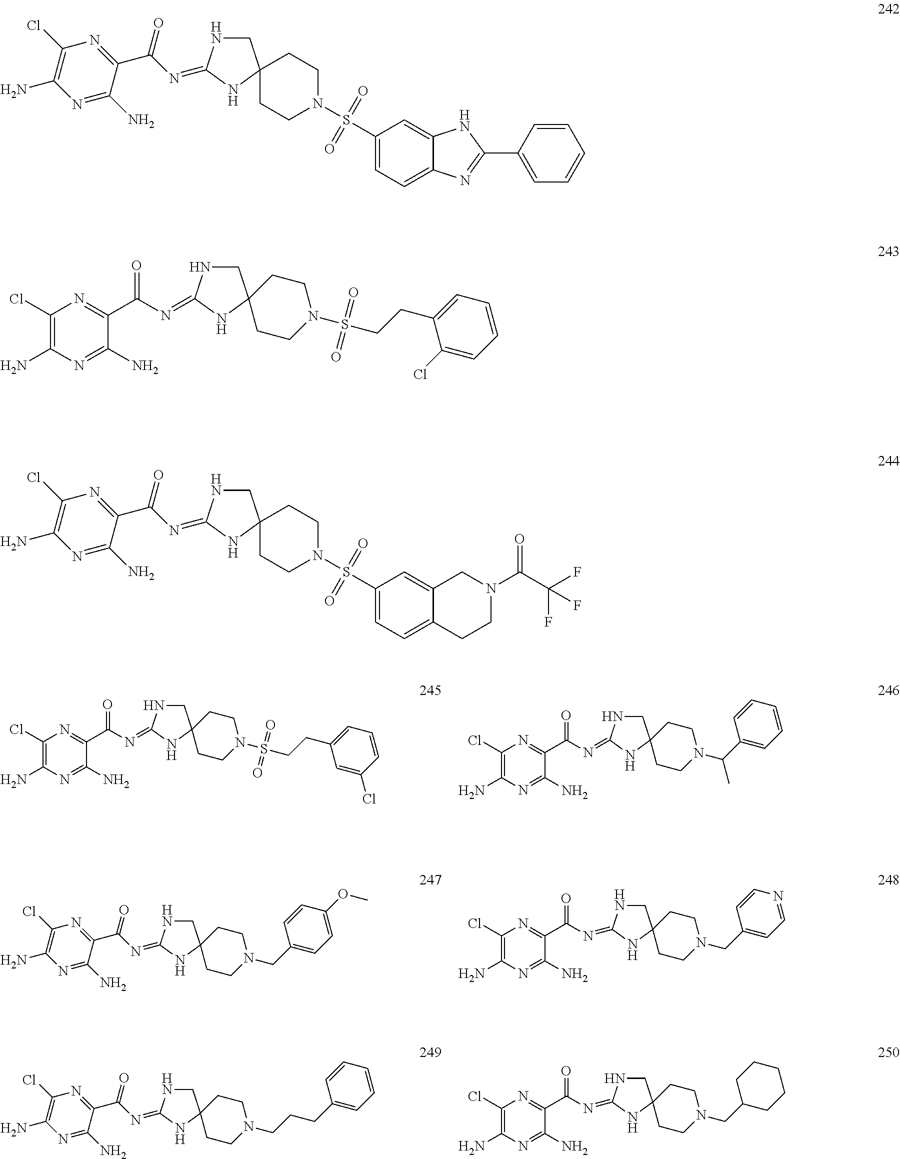

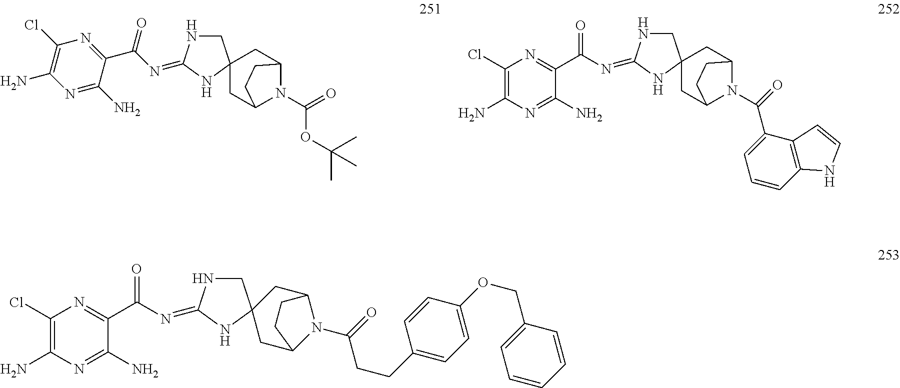 [SMILES: CC(C)(C)OC(=O)N1C2CCC1CC1(CN/C(=N\C(=O)c3nc(Cl)c(N)nc3N)N1)C2 ; Nc1nc(N)c(C(=O)/N=C2\NCC3(CC4CCC(C3)N4C(=O)c3cccc4[nH]ccc34)N2)nc1Cl ; Nc1nc(N)c(C(=O)/N=C2\NCC3(CC4CCC(C3)N4C(=O)CCc3ccc(OCc4ccccc4)cc3)N2)nc1Cl]

### Definitions for Compounds of Formula E

Terms used in the specification have the following meanings:

“Optionally substituted” means the group referred to can be substituted at one or more positions by any one or any combination of the radicals listed thereafter.

“optionally substituted by one or more Z groups” denotes that the relevant group may include one or more substituents, each independently selected from the groups included within the definition of Z. Thus, where there are two or more Z group substituents, these may be the same or different.

“Halo” or “halogen”, as used herein, may be fluorine, chlorine, bromine or iodine.

“C₁-C₈-Alkyl”, as used herein, denotes straight chain or branched alkyl having 1-8 carbon atoms. If a different number of carbon atoms is specified, such as C₆ or C₃, then the definition is to be amended accordingly.

“C₁-C₈-Alkoxy”, as used herein, denotes straight chain or branched alkoxy having 1-8 carbon atoms. If a different number of carbon atoms is specified, such as C₆ or C₃, then the definition is to be amended accordingly.

The term “alkylene” denotes a straight chain or branched saturated hydrocarbon chain containing between 1 and 8 carbon atoms. If a different number of carbon atoms is specified, such as C₆ or C₃, then the definition is to be amended accordingly.

“Amino-C₁-C₈-alkyl” and “amino-C₁-C₈-alkoxy” denote amino attached by a nitrogen atom to C₁-C₈-alkyl, e.g., NH₂—(C₁-C₈)—, or to C₁-C₈-alkoxy, e.g., NH₂—(C₁-C₈)—O—. If a different number of carbon atoms is specified, such as C₆ or C₃, then the definition is to be amended accordingly.

“C₁-C₈-Alkylamino” and “di(C₁-C₈-alkyl)amino” denote C₁-C₈-alkyl, as hereinbefore defined, attached by a carbon atom to an amino group. The C₁-C₈-alkyl groups in di(C₁-C₈-alkyl)amino may be the same or different. If a different number of carbon atoms is specified, such as C₆ or C₃, then the definition is to be amended accordingly.

“Amino-(hydroxy)-C₁-C₈-alkyl” denotes amino attached by a nitrogen atom to C₁-C₈-alkyl and hydroxy attached by an oxygen atom to the same C₁-C₈-alkyl. If a different number of carbon atoms is specified, such as C₆ or C₃, then the definition is to be amended accordingly.

“C₁-C₈-Alkylcarbonyl” and “C₁-C₈-alkoxycarbonyl”, as used herein, denote C₁-C₈-alkyl or C₁-C₈-alkoxy, respectively, as hereinbefore defined, attached by a carbon atom to a carbonyl group. If a different number of carbon atoms is specified, such as C₆ or C₃, then the definition is to be amended accordingly.

“C₃-C₈-Cycloalkylcarbonyl”, as used herein, denotes C₃-C₈-cycloalkyl, as hereinbefore defined, attached by a carbon atom to a carbonyl group. If a different number of carbon atoms is specified, such as C₆ or C₃, then the definition is to be amended accordingly.

“C₇-C₁₄-Aralkyl”, as used herein, denotes alkyl, e.g., C₁-C₄-alkyl, as hereinbefore defined, substituted by a C₆-C₁₀-aromatic carbocyclic group, as herein defined. If a different number of carbon atoms is specified, such as C₆ or C₃, then the definition is to be amended accordingly.

“C₃-C₁₅-Carbocyclic group”, as used herein, denotes a carbocyclic group having 3- to 15-ring carbon atoms that is saturated or partially saturated, such as a C₃-C₈-cycloalkyl. Examples of C₃-C₁₅-carbocyclic groups include but are not limited to cyclopropyl, cyclobutyl, cyclopentyl, cyclohexyl, cycloheptyl or cyclooctyl or a bicyclic group, such as bicyclooctyl; bicyclononyl including indanyl and indenyl and bicyclodecyl. If a different number of carbon atoms is specified, such as C₆, then the definition is to be amended accordingly.

“aryl” or “C₆-C₁₅-Aromatic carbocyclic group”, as used herein, denotes an aromatic group having 6- to 15-ring carbon atoms. Examples of C₆-C₁₅-aromatic carbocyclic groups include, but are not limited to, phenyl, phenylene, benzenetriyl, naphthyl, naphthylene, naphthalenetriyl or anthrylene. If a different number of carbon atoms is specified, such as C₁₀, then the definition is to be amended accordingly.

“4- to 8-Membered heterocyclic group”, “5- to 6-membered heterocyclic group”, “3- to 10-membered heterocyclic group”, “3- to 14-membered heterocyclic group”, “4- to 14-membered heterocyclic group” and “5- to 14-membered heterocyclic group”, refers, respectively, to 4- to 8-membered, 5- to 6-membered, 3- to 10-membered, 3- to 14-membered, 4- to 14-membered and 5- to 14-membered heterocyclic rings containing at least one ring heteroatom selected from the group consisting of nitrogen, oxygen and sulphur, which may be saturated, partially saturated or unsaturated (aromatic). The heterocyclic group includes single ring groups, fused ring groups and bridged groups. Examples of such heterocyclic groups include, but are not limited to, furan, pyrrole, pyrrolidine, pyrazole, imidazole, triazole, isotriazole, tetrazole, thiadiazole, isothiazole, oxadiazole, pyridine, piperidine, pyrazine, oxazole, isoxazole, pyrazine, pyridazine, pyrimidine, piperazine, pyrrolidine, pyrrolidinone, morpholine, triazine, oxazine, tetrahyrofuran, tetrahydrothiophene, tetrahydrothiopyran, tetrahydropyran, 1,4-dioxane, 1,4-oxathiane, indazole, quinoline, indazole, indole, 8-aza-bicyclo[3.2.1]octane or thiazole.

A second aspect of the present invention provides for the use of a compound of Formula E in any of the aforementioned embodiments, in free or pharmaceutically acceptable salt form, for the manufacture of a medicament for the treatment of an inflammatory or allergic condition, particularly an inflammatory or obstructive airways disease or mucosal hydration.

An embodiment of the present invention provides for the use of a compound of Formula E in any of the aforementioned embodiments, in free or pharmaceutically acceptable salt form, for the manufacture of a medicament for the treatment of an inflammatory or allergic condition selected from cystic fibrosis, primary ciliary dyskinesia, chronic bronchitis, chronic obstructive pulmonary disease, asthma, respiratory tract infections, lung carcinoma, xerostomia and keratoconjunctivitis sire.

It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments. It is understood by those skilled in the art that combinations of substituents where not possible are not an aspect of the present invention.

Throughout this specification and in the claims that follow, unless the context requires otherwise, the word “comprise”, or variations, such as “comprises” or “comprising”, will be understood to imply the inclusion of a stated integer or step or group of integers or steps but not the exclusion of any other integer or step or group of integers or steps.

Especially preferred specific compounds of Formula E are those described hereinafter in the Examples.

The compounds represented by Formula E may be capable of forming acid addition salts, particularly pharmaceutically acceptable acid addition salts. Pharmaceutically acceptable acid addition salts of the compound of Formula E include those of inorganic acids, e.g., hydrohalic acids, such as hydrofluoric acid, hydrochloric acid, hydrobromic acid or hydroiodic acid, nitric acid, sulfuric acid, phosphoric acid; and organic acids, e.g., aliphatic monocarboxylic acids, such as formic acid, acetic acid, trifluoroacetic acid, propionic acid and butyric acid; aliphatic hydroxy acids, such as lactic acid, citric acid, tartaric acid or malic acid; dicarboxylic acids, such as maleic acid or succinic acid; aromatic carboxylic acids, such as benzoic acid, p-chlorobenzoic acid, diphenylacetic acid, para-biphenyl benzoic acid or triphenylacetic acid; aromatic hydroxy acids, such as o-hydroxybenzoic acid, p-hydroxybenzoic acid, 1-hydroxynaphthalene-2-carboxylic acid or 3-hydroxynaphthalene-2-carboxylic acid; cinnamic acids, such as 3-(2-naphthalenyl)propenoic acid, para-methoxy cinnamic acid or para-methyl cinnamic acid; and sulfonic acids, such as methanesulfonic acid or benzenesulfonic acid. These salts may be prepared from compounds of Formula E by known salt-forming procedures.

Compounds of Formula E which may contain acidic, e.g., carboxyl, groups, are also capable of forming salts with bases, in particular, pharmaceutically acceptable bases, such as those well-known in the art; suitable such salts include metal salts, particularly alkali metal or alkaline earth metal salts, such as sodium, potassium, magnesium or calcium salts; or salts with ammonia or pharmaceutically acceptable organic amines or heterocyclic bases, such as ethanolamines, benzylamines or pyridine. These salts may be prepared from compounds of Formula E by known salt-forming procedures.

Stereoisomers are those compounds where there is an asymmetric carbon atom. The compounds exist in individual optically active isomeric forms or as mixtures thereof, e.g., as diastereomeric mixtures. The present invention embraces both individual optically active R and S isomers, as well as mixtures thereof. Individual isomers can be separated by methods well-known to those skilled in the art, e.g., chiral high performance liquid chromatography (HPLC).

Tautomers are one of two or more structural isomers that exist in equilibrium and are readily converted from one isomeric form to another.

More specifically, for example, compounds of Formula Ea where ER⁶ and/or ER¹¹ are

hydrogen may exist in one or both of the following tautomeric forms:

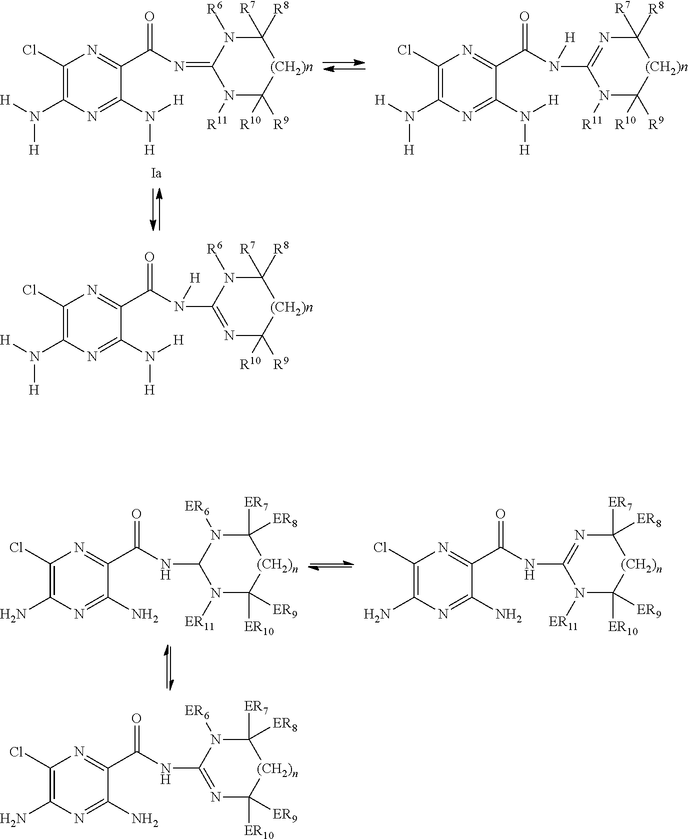

Compounds according to Formula E may exist in corresponding tautomeric forms.

Examples of tautomers include but are not limited to those compounds defined in the claims.

The compounds of the invention may exist in both unsolvated and solvated forms. The term “solvate” is used herein to describe a molecular complex comprising the compound of the invention and one or more pharmaceutically acceptable solvent molecules, e.g., ethanol. The term “hydrate” is employed when said solvent is water.

### Synthesis

Generally, compounds according to Formula E can be synthesized by the routes described in Scheme 1 and the Examples.

For instance, intermediate 1 can be reacted with intermediate 2 in an organic solvent to provide compound 3 which can be isolated as the free base. The free base can then be converted to a salt form by treatment with an appropriate acid.

Intermediates can be prepared from methods known by those skilled in the art or are commercially available.

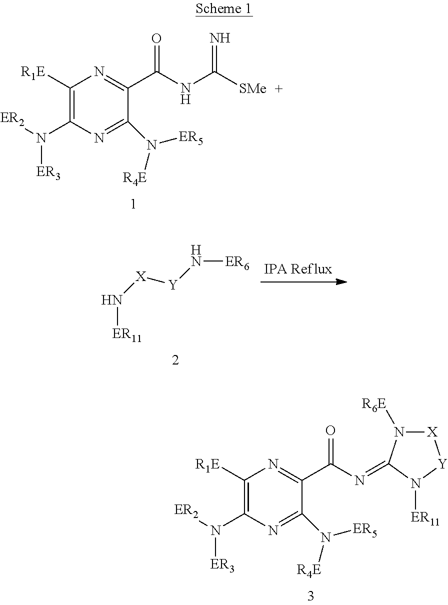

In Scheme 1, ER¹, ER², ER³, ER⁴, ER⁵, ER⁶ and ER¹¹ are as defined above; Y is CER⁷ER⁸; X is CER⁹ER¹⁰; n is 0; and ER⁷, ER⁸, ER⁹ and ER¹⁰ are also as defined above. For compounds where n is 1 or 2, then the appropriate methylene or ethylene linking groups are inserted between X and Y in the diamine reactant 2.

The compounds of Formula E and Formula E2 above can be prepared according to conventional routes described in the literature.

Compounds of Formula E, in free form, may be converted into salt form, and vice versa, in a conventional manners understood by those skilled in the art. The compounds in free or salt form can be obtained in the form of hydrates or solvates containing a solvent used for crystallization. Compounds of Formula E can be recovered from reaction mixtures and purified in a conventional manner. Isomers, such as stereoisomers, may be obtained in a conventional manner, e.g., by fractional crystallisation or asymmetric synthesis from correspondingly asymmetrically substituted, e.g., optically active, starting materials. The compounds of Formula E can be prepared, e.g., using the reactions and techniques described below and in the Examples. The reactions may be performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of Formula E into another compound of Formula E. Examples of such manipulations are conversion of an ester or a ketone to an alcohol; conversion of an ester to a ketone; interconversions of esters, acids and amides; alkylation, acylation and sulfonylation of alcohols and amines; and many others. Substituents can also be added using common reactions, such as alkylation, acylation, halogenation or oxidation. Such manipulations are well-known in the art, and many reference works summarize procedures and methods for such manipulations. Some reference works which gives examples and references to the primary literature of organic synthesis for many functional group manipulations, as well as other transformations commonly used in the art of organic synthesis are March's Organic Chemistry, 5^{th} Edition, Wiley and Chichester, Eds. (2001); Comprehensive Organic Transformations, Larock, Ed., VCH (1989); Comprehensive Organic Functional Group Transformations, Katritzky et al. (series editors), Pergamon (1995); and Comprehensive Organic Synthesis, Trost and Fleming (series editors), Pergamon (1991). It will also be recognized that another major consideration in the planning of any synthetic route in this field is the judicious choice of the protecting group used for protection of the reactive functional groups present in the compounds described in this invention. Multiple protecting groups within the same molecule can be chosen such that each of these protecting groups can either be removed without removal of other protecting groups in the same molecule, or several protecting groups can be removed using the same reaction step, depending upon the outcome desired. An authoritative account describing many alternatives to the trained practitioner is Greene and Wuts, Protective Groups in Organic Synthesis, Wiley and Sons (1999).

### Pharmacological Activity

Having regard to their blockade of the epithelial sodium channel (ENaC), compounds of Formula E, in free or pharmaceutically acceptable salt form, hereinafter alternately referred to as “agents of the invention”, are useful in the treatment of conditions which respond to the blockade of the epithelial sodium channel, particularly conditions benefiting from mucosal hydration.

Diseases mediated by blockade of the epithelial sodium channel, include diseases associated with the regulation of fluid volumes across epithelial membranes. For example, the volume of airway surface liquid is a key regulator of mucociliary clearance and the maintenance of lung health. The blockade of the epithelial sodium channel will promote fluid accumulation on the mucosal side of the airway epithelium thereby promoting mucus clearance and preventing the accumulation of mucus and sputum in respiratory tissues (including lung airways). Such diseases include respiratory diseases, such as cystic fibrosis, primary ciliary dyskinesia, chronic bronchitis, chronic obstructive pulmonary disease (COPD), asthma, respiratory tract infections (acute and chronic; viral and bacterial) and lung carcinoma. Diseases mediated by blockade of the epithelial sodium channel also include diseases other than respiratory diseases that are associated with abnormal fluid regulation across an epithelium, perhaps involving abnormal physiology of the protective surface liquids on their surface, e.g., xerostomia (dry mouth) or keratoconjunctivitis sire (dry eye). Furthermore, blockade of the epithelial sodium channel in the kidney could be used to promote diuresis and thereby induce a hypotensive effect.

Treatment in accordance with the invention may be symptomatic or prophylactic.

Asthma includes both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., cortico-steroid) or bronchodilatory. Prophylactic benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 am, i.e., at a time normally substantially distant from any previously administered symptomatic asthma therapy.

Chronic obstructive pulmonary disease includes chronic bronchitis or dyspnea associated therewith, emphysema, as well as exacerbation of airways hyperreactivity consequent to other drug therapy, in particular, other inhaled drug therapy. The invention is also applicable to the treatment of bronchitis of whatever type or genesis including, e.g., acute, arachidic, catarrhal, croupus, chronic or phthinoid bronchitis.

The agents of the invention may also be useful as acid-sensing ion channel (ASIC) blockers. Thus they may be useful in the treatment of conditions which respond to the blockade of the acid-sensing ion channel.

The suitability of epithelial sodium channel blocker as a treatment of a disease benefiting from mucosal hydration, may be tested by determining the inhibitory effect of the channel blocker on ENaC in a suitable cell-based assay. For example single cells or confluent epithelia, endogenously expressing or engineered to over express ENaC can be used to assess channel function using electrophysiological techniques or ion flux studies. See methods described in: Hirsh et al., J Pharm Exp Ther (2004); Moody et al., Am J Physiol Cell Physiol (2005).

Epithelial sodium channel blockers, including the compounds of formula (I), are also useful as co-therapeutic agents for use in combination with other drug substances, such as anti-inflammatory, bronchodilatory, antihistamine or anti-tussive drug substances, particularly in the treatment of cystic fibrosis or obstructive or inflammatory airways diseases such as those mentioned hereinbefore, e.g., as potentiators of therapeutic activity of such drugs or as a means of reducing required dosaging or potential side effects of such drugs.

The epithelial sodium channel blocker may be mixed with the other drug substance in a fixed pharmaceutical composition or it may be administered separately, before, simultaneously with or after the other drug substance.

Accordingly, the invention includes as a further aspect a combination of ENaC inhibitor and an CF Modulator modulator selected from at least one of Columns A, B, C, or D, optionally, with osmotic agents (hypertonic saline, dextran, mannitol, Xylitol)+modifiers of CFTR function, both wild-type and mutant (correctors+potentiators), e.g., those described in WO 2007/021982, WO 2006/099256, WO 2006/127588, WO 2004/080972, WO 2005/026137, WO 2005/035514, WO 2005/075435, WO 2004/111014, WO 2006/101740, WO 2004/110352, WO 2005/120497 and US 2005/0176761, an anti-inflammatory, bronchodilatory, antihistamine, anti-tussive, antibiotic or DNase drug substance, said epithelial sodium channel blocker and said drug substance being in the same or different pharmaceutical composition.

Suitable antibiotics include macrolide antibiotics, e.g., tobramycin (TOBI™).

Suitable DNase drug substances include dornase alfa (Pulmozyme™), a highly-purified solution of recombinant human deoxyribonuclease I (rhDNase), which selectively cleaves DNA. Dornase alfa is used to treat cystic fibrosis.

Other useful combinations of epithelial sodium channel blockers with anti-inflammatory drugs are those with antagonists of chemokine receptors, e.g., CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists, such as Schering-Plough antagonists SC-351125, SCH-55700 and SCH-D; Takeda antagonists, such as N-[[4-[[[6,7-dihydro-2-(4-methyl-phenyl)-5H-benzo-cyclohepten-8-yl]carbonyl]amino]phenyl]-methyl]tetrahydro-N,N-dimethyl-2H-pyran-4-amin-ium chloride (TAK-770); and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

Suitable anti-inflammatory drugs include steroids, in particular, glucocorticosteroids, such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTD4 antagonists, such as montelukast and zafirlukast; PDE4 inhibitors, such as cilomilast (Ariflo® GlaxoSmithKline), Roflumilast (Byk Gulden),V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; adenosine A2B receptor antagonists such as those described in WO 02/42298; and beta-2 adrenoceptor agonists, such as albuterol (salbutamol), metaproterenol, terbutaline, salmeterol fenoterol, procaterol, and especially, formoterol, carmoterol and pharmaceutically acceptable salts thereof, and compounds (in free or salt or solvate form) of formula (I) of WO 0075114, which document is incorporated herein by reference, preferably compounds of the Examples thereof, especially a compound of formula:

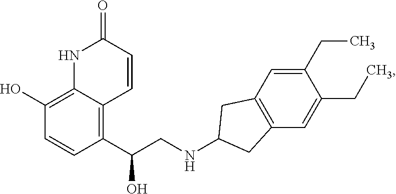 [SMILES: CCc1cc2c(cc1CC)CC(NC[C@@H](O)c1ccc(O)c3[nH]c(=O)ccc13)C2]

corresponding to indacaterol and pharmaceutically acceptable salts thereof, as well as compounds (in free or salt or solvate form) of Formula E of WO 04/16601, and also compounds of EP 1440966, JP 05025045, WO 93/18007, WO 99/64035, USP 2002/0055651, WO 01/42193, WO 01/83462, WO 02/66422, WO 02/70490, WO 02/76933, WO 03/2-[439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618, WO 04/46083, WO 04/80964, WO 04/108765 and WO 04/108676.

Suitable bronchodilatory drugs include anticholinergic or antimuscarinic agents, in particular, ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, WO 01/04118, WO 02/00652, WO 02/51841, WO 02/53564, WO 03/00840, WO 03/33495, WO 03/53966, WO 03/87094, WO 04/018422 and WO 04/05285.

Suitable dual anti-inflammatory and bronchodilatory drugs include dual beta-2 adrenoceptor agonist/muscarinic antagonists such as those disclosed in USP 2004/0167167, WO 04/74246 and WO 04/74812.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine, as well as those disclosed in JP 2004107299, WO 03/099807 and WO 04/026841.

In accordance with the foregoing, the invention also provides as a further aspect a method for the treatment of a condition responsive to blockade of the epithelial sodium channel, e.g., diseases associated with the regulation of fluid volumes across epithelial membranes, particularly an obstructive airways disease, which comprises administering to a subject, particularly a human subject, in need thereof a compound of Formula E, in free form or in the form of a pharmaceutically acceptable salt in combination with an ABC transporter modulator component of any one of Columns A, B, C, or D.

In another aspect the invention provides a compound of Formula E, in free form or in the form of a pharmaceutically acceptable salt in combination with an ABC transporter modulator component of any one of Columns A, B, C, or D, for use in the manufacture of a medicament for the treatment of a condition responsive to blockade of the epithelial sodium channel, particularly an obstructive airways disease, e.g., cystic fibrosis and COPD.

The agents of the invention may be administered by any appropriate route, e.g. orally, e.g., in the form of a tablet or capsule; parenterally, e.g., intravenously; by inhalation, e.g., in the treatment of an obstructive airways disease; intranasally, e.g., in the treatment of allergic rhinitis; topically to the skin; or rectally. In a further aspect, the invention also provides a pharmaceutical composition comprising a compound of Formula E, in free form or in the form of a pharmaceutically acceptable salt, optionally together with a pharmaceutically acceptable diluent or carrier. In some embodiments, the pharmaceutical composition can include a compound of Formula E, in combination with at least one ABC transporter modulator from Columns A, B, C, or D. The composition may contain a co-therapeutic agent, such as an anti-inflammatory, broncho-dilatory, antihistamine or anti-tussive drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g., patches. Compositions for inhalation may comprise aerosol or other atomizable formulations or dry powder formulations.

When the composition comprises an aerosol formulation, it preferably contains, e.g., a hydro-fluoro-alkane (HFA) propellant, such as HFA134a or HFA227 or a mixture of these, and may contain one or more co-solvents known in the art, such as ethanol (up to 20% by weight), and/or one or more surfactants, such as oleic acid or sorbitan trioleate, and/or one or more bulking agents, such as lactose. When the composition comprises a dry powder formulation, it preferably contains, e.g., the compound of Formula E having a particle diameter up to 10 microns, optionally together with a diluent or carrier, such as lactose, of the desired particle size distribution and a compound that helps to protect against product performance deterioration due to moisture, e.g., magnesium stearate. When the composition comprises a nebulised formulation, it preferably contains, e.g., the compound of Formula E either dissolved, or suspended, in a vehicle containing water, a co-solvent, such as ethanol or propylene glycol and a stabilizer, which may be a surfactant.

Further aspects of the invention include:

a compound of Formula E in inhalable form, e.g., in an aerosol or other atomisable composition or in inhalable particulate, e.g., micronised form;

an inhalable medicament comprising a compound of Formula E in inhalable form;

a pharmaceutical product comprising a compound of Formula E in inhalable form in association with an inhalation device; and an inhalation device containing a compound of Formula E in inhalable form.

Dosages of compounds of Formula E employed in practicing the present invention will of course vary depending, e.g., on the particular condition to be treated, the effect desired and the mode of administration. In general, suitable daily dosages for administration by inhalation are of the order of 0.005 to about 100 mg, for example, from about 0.01 to about 50 mg, or from about 0.1 to about 30 mg while for oral administration suitable daily doses are of the order of 0.05 to about 200 mg, or from about 0.1 to about 180 mg, or from about 0.1 to about 160 mg, or from about 0.1 to about 140 mg or from about 0.1 to about 120 mg, or from about 0.1 to about 100 mg or from about 0.1 to about 80 mg or from about 0.1 to about 60 mg, or from about 0.1 to about 40 mg, or from about 0.1 to about 20 mg, or from about 0.01 to about 200 mg, or from about 0.1 to about 180 mg, or from about 0.5 to about 180 mg, or from about 1 to about 180 mg, or from about 10 to about 180 mg, or from about 20 to about 180 mg, or from about 30 to about 180 mg, or from about 40 to about 180 mg, or from about 50 to about 180 mg, or from about 60 to about 180 mg, or from about 70 to about 180 mg, or from about 80 to about 180 mg, or from about 90 to about 180 mg, or from about 100 to about 180 mg, or from about 110 to about 180 mg, or from about 120 to about 180 mg, or from about 130 to about 180 mg, or from about 140 to about 180 mg, or from about 150 to about 180 mg, or from about 160 to about 180 mg, or from about 15 to about 175 mg, or from about 25 to about 150 mg, or from about 50 to about 125 mg, or from about 75 to about 100 mg. As used herein, dosage ranges are provided merely for exemplary amounts, and all values within the stated ranges are also contemplated and may be determined using ordinary skill in the medical art given the customary factors used to calculate or titrate a dose of a drug for a given patients. Exemplary factors which may be used in the determination of an appropriate dose can include the disorder being treated and the severity of the disorder; the activity of the composition employed; the specific composition employed; the age, body weight, general health, sex and diet of the patient; the time of administration, route of administration, and rate of excretion of the specific composition employed; the duration of the treatment; drugs used in combination or coincidental with the specific composition employed, and like factors well known in the medical arts.

### Pharmaceutical Use and Assay

Compounds of Formula E and their pharmaceutically acceptable salts, hereinafter referred to alternatively as “illustrative ENaC inhibitors of the invention”, are useful as pharmaceuticals. In particular, the compounds have good ENaC blocker activity and may be tested in the following assays.

### Cell Culture

Human Bronchial Epithelial cells (HBECs) (Cambrex) were cultured under air-liquid interface conditions to provide a well differentiated mucociliary phenotype.

HBECs were cultured using a modification of the method described by Gray and colleagues (Gray et al., 1996). Cells were seeded in plastic T-162 flasks and were grown in bronchial epithelial cell growth medium (BEGM; Cambrex) supplemented with bovine pituitary extract (52 [μg/mL), hydrocortisone (0.5 [μg/mL), human recombinant epidermal growth factor (0.5 ng/mL), epinephrine (0.5 [μg/mL), transferrin (10 μg/mL), insulin (5 μg/mL), retinoic acid (0.1 μg/mL), triiodothyronine (6.5 μg/mL), gentamycin (50 μg/mL) and amphotericin B (50 ng/mL). Medium was changed every 48 hours until cells were 90% confluent. Cells were then passaged and seeded (8.25×10⁵ cells/insert) on polycarbonate Snapwell inserts (Costar) in differentiation media containing 50% DMEM in BEGM with the same supplements as above but without triiodothyronine and a final retinoic acid concentration of 50 nM (all-trans retinoic acid). Cells were maintained submerged for the first 7 days in culture, after which time they were exposed to an apical air interface for the remainder of the culture period. At this time, media was changed to DMEM:F12 media containing 2% v/v Ultroser G for the remainder of culture. Amphotericin B was removed from all media 3 feeds prior to use in the Using Chambers. Cells were used between days 7 and 21 after establishment of the apical-air interface. At all stages of culture, cells were maintained at 37° C. in 5% CO₂ in an air incubator.

### Short Circuit Current (ISC) Measurements

Snapwell inserts were mounted in Vertical Diffusion Chambers (Costar) and were bathed with continuously gassed Ringer solution (5% CO₂ in O₂; pH 7.4) maintained at 37° C. containing (in mM): 120 NaCl, 25 NaHCO₃, 3.3 KH₂PO₄, 0.8 K₂HPO₄, 1.2 CaCl₂, 1.2 MgCl₂, and 10 glucose. The solution osmolarity was between 280 and 300 mOsmol/kg H₂O for all physiological salt solutions used. Cells were voltage clamped to 0 mV (model EVC4000; WPI). RT was measured by applying a 1- or 2-mV pulse at 30-s intervals and calculating RT by Ohm's law. Data were recorded using a PowerLab workstation (ADInstruments).

Test compounds were prepared as a 10 mM stock solution in DMSO (95%). Serial 3-fold dilutions were freshly prepared in an appropriate vehicle (distilled H₂O or Ringers solution). The initial concentration was added to the apical chamber as a 1000× concentrate in 5 μL, resulting in a final 1× concentration the 5 mL volume of the Using chamber. Subsequent additions of compound were added in a 3.3 μL volume of the 1000× serially diluted stock solution. At the completion of the concentration-response experiment, amiloride (10 μM) was added into the apical chamber to enable the total amiloride-sensitive current to be measured. An amiloride control IC₅₀ was established at the start of each experiment.

Results are expressed as the mean % inhibition of the amiloride-sensitive ISC. Concentration-response curves were plotted and IC₅₀ values generated using GraphPad Prism 3.02. Cell inserts were typically run in duplicate and the IC₅₀ calculated on the mean % inhibition data.

Compounds of the Examples, herein below, generally have IC₅₀ values in the data measurements described above below 10 μM. For example, the compounds of the Examples shown below have the indicated IC₅₀ values.

| | IC₅₀ | | IC₅₀ |
|---|---|---|---|
| EX | (μM) | EX | (μM) |
| 5 | 0.065 | 70 | 0.074 |
| 11 | 1.686 | 71 | 0.042 |
| 19 | 0.018 | 76 | 0.012 |
| 23 | 0.0335 | 86 | 0.008 |
| 25 | 0.270 | 91 | 0.0885 |
| 26 | 0.011 | 94 | 0.009 |
| 29 | 0.005 | 96 | 0.037 |
| 32 | 0.018 | 99 | 0.019 |
| 34 | 0.095 | 118 | 0.175 |
| 35 | 0.031 | 126 | 0.025 |
| 39 | 0.0055 | 128 | 0.0115 |
| 40 | 0.0055 | 141 | 0.002 |
| 41 | 0.0095 | 146 | 0.006 |
| 42 | 0.011 | 147 | 0.016 |
| 43 | 0.013 | 185 | 0.062 |
| 44 | 0.0295 | 215 | 0.036 |
| 45 | 0.0426 | 220 | 0.0085 |
| 48 | 0.0165 | 228 | 0.0935 |
| 58 | 0.143 | 232 | 0.054 |
| 61 | 0.3465 | 235 | 0.364 |
| 62 | 0.013 | 238 | 0.0119 |
| 64 | 0.0255 | 246 | 0.025 |
| 65 | 0.0395 | 252 | 0.028 |

The invention is illustrated by the following Examples.

### EXAMPLES

Compounds of Formula Eb are shown in Table II.E-1.

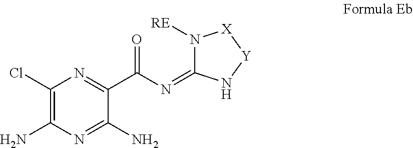

Methods for preparing such compounds are described hereinafter. The table also shows mass spectrometry [M+H]⁺ data.

TABLE II.E-1

| Ex- | | M/s |
|---|---|---|
| ample | Structure | [M + H]⁺ |
| 1 | 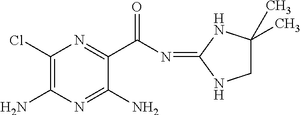 [SMILES: CC1(C)CN/C(=N/C(=O)c2nc(Cl)c(N)nc2N)N1] | 284 |
| 2 | 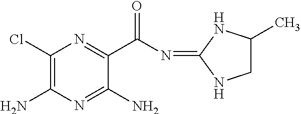 [SMILES: CC1CN/C(=N/C(=O)c2nc(Cl)c(N)nc2N)N1] | 270 |
| 3 | 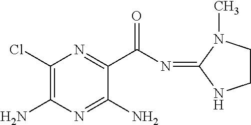 [SMILES: CN1CCN/C1=N/C(=O)c1nc(Cl)c(N)nc1N] | 270 |
| 4 | 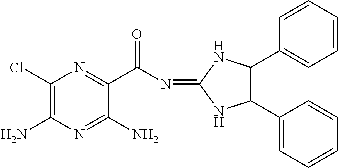 [SMILES: Nc1nc(N)c(C(=O)N=C2NC(c3ccccc3)C(c3ccccc3)N2)nc1Cl] | 408 |
| 5 | 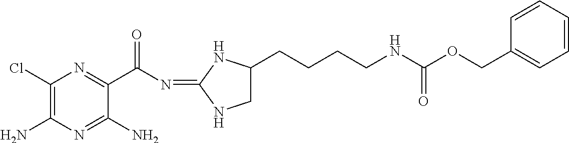 [SMILES: Nc1nc(N)c(C(=O)/N=C2/NCC(CCCCNC(=O)OCc3ccccc3)N2)nc1Cl] | 461 |
| 6 | 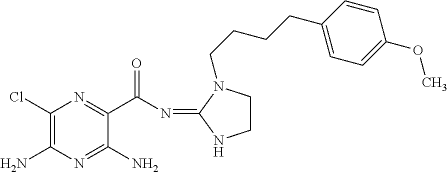 [SMILES: COc1ccc(CCCCN2CCN/C2=N/C(=O)c2nc(Cl)c(N)nc2N)cc1] | 418 |
| 7 | 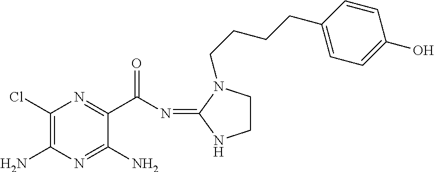 [SMILES: Nc1nc(N)c(C(=O)/N=C2/NCCN2CCCCc2ccc(O)cc2)nc1Cl] | 404 |
| 8 | 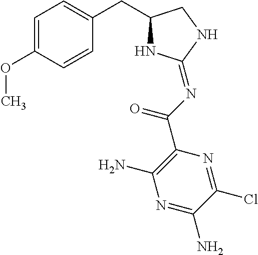 [SMILES: COc1ccc(C[C@H]2CN/C(=N/C(=O)c3nc(Cl)c(N)nc3N)N2)cc1] | 376/378 |
| 9 | 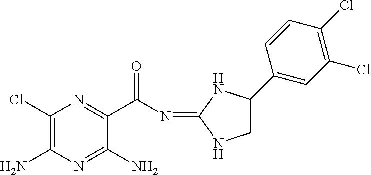 [SMILES: Nc1nc(N)c(C(=O)/N=C2/NCC(c3ccc(Cl)c(Cl)c3)N2)nc1Cl] | 400 |
| 10 | 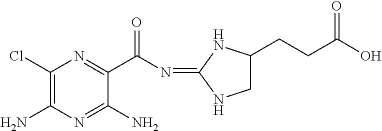 [SMILES: Nc1nc(N)c(C(=O)/N=C2/NCC(CCC(=O)O)N2)nc1Cl] | 328 |
| 11 | 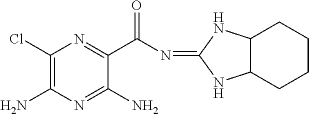 [SMILES: Nc1nc(N)c(C(=O)N=C2NC3CCCCC3N2)nc1Cl] | 310 |
| 12 | 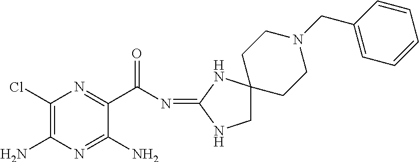 [SMILES: Nc1nc(N)c(C(=O)/N=C2/NCC3(CCN(Cc4ccccc4)CC3)N2)nc1Cl] | 415 |
| 13 | 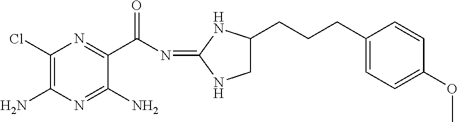 [SMILES: COc1ccc(CCCC2CN/C(=N/C(=O)c3nc(Cl)c(N)nc3N)N2)cc1] | 404 |
| 14 | 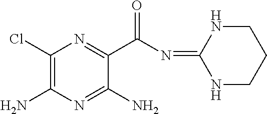 [SMILES: Nc1nc(N)c(C(=O)N=C2NCCCN2)nc1Cl] | 270 |
| 15 | 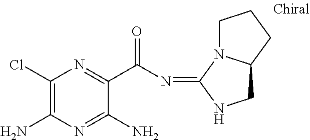 [SMILES: Nc1nc(N)c(C(=O)/N=C2/NC[C@@H]3CCCN23)nc1Cl] | 296 |
| 16 | 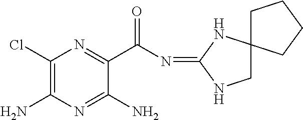 [SMILES: Nc1nc(N)c(C(=O)/N=C2/NCC3(CCCC3)N2)nc1Cl] | 310 |
| 17 | 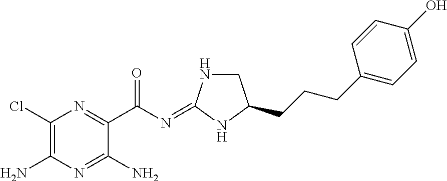 [SMILES: Nc1nc(N)c(C(=O)/N=C2\NC[C@@H](CCCc3ccc(O)cc3)N2)nc1Cl] | 390 |
| 18 | 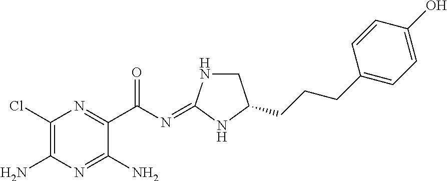 [SMILES: Nc1nc(N)c(C(=O)/N=C2\NC[C@H](CCCc3ccc(O)cc3)N2)nc1Cl] | 390 |
| 19 | 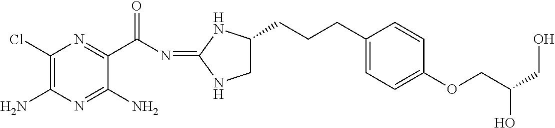 [SMILES: Nc1nc(N)c(C(=O)/N=C2/NC[C@@H](CCCc3ccc(OC[C@@H](O)CO)cc3)N2)nc1Cl] | 464 |
| 20 | 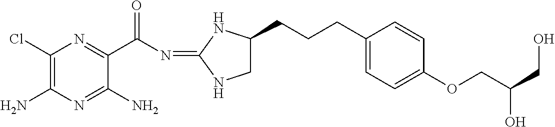 [SMILES: Nc1nc(N)c(C(=O)/N=C2/NC[C@H](CCCc3ccc(OC[C@H](O)CO)cc3)N2)nc1Cl] | 464 |
| 21 | 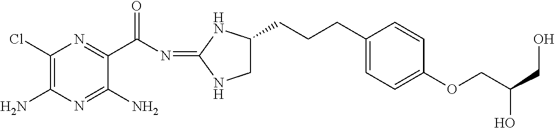 [SMILES: Nc1nc(N)c(C(=O)/N=C2/NC[C@@H](CCCc3ccc(OC[C@H](O)CO)cc3)N2)nc1Cl] | 464 |
| 22 | 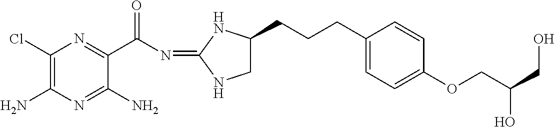 [SMILES: Nc1nc(N)c(C(=O)/N=C2/NC[C@H](CCCc3ccc(OC[C@H](O)CO)cc3)N2)nc1Cl] | 464 |
| 23 | 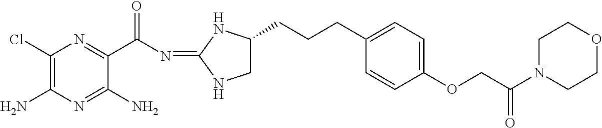 [SMILES: Nc1nc(N)c(C(=O)/N=C2/NC[C@@H](CCCc3ccc(OCC(=O)N4CCOCC4)cc3)N2)nc1Cl] | 517 |
| 24 | 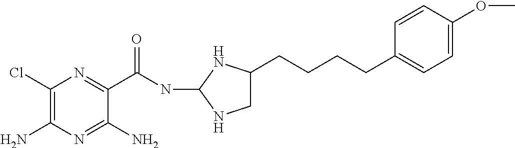 [SMILES: COc1ccc(CCCCC2CNC(NC(=O)c3nc(Cl)c(N)nc3N)N2)cc1] | 418.2 |
| 25 | 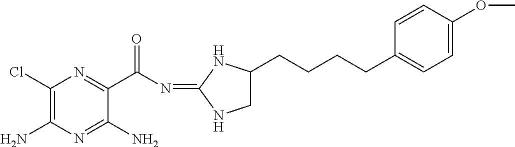 [SMILES: COc1ccc(CCCCC2CN/C(=N/C(=O)c3nc(Cl)c(N)nc3N)N2)cc1] | 418.2 |
| 26 | 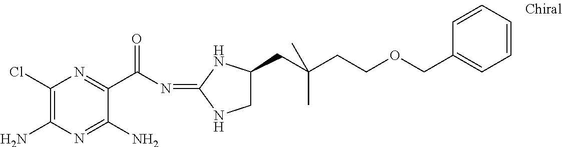 [SMILES: CC(C)(CCOCc1ccccc1)C[C@H]1CN/C(=N/C(=O)c2nc(Cl)c(N)nc2N)N1] | 446 |
| 27 | 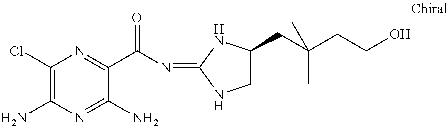 [SMILES: CC(C)(CCO)C[C@H]1CN/C(=N/C(=O)c2nc(Cl)c(N)nc2N)N1] | 356 |
| 28 | 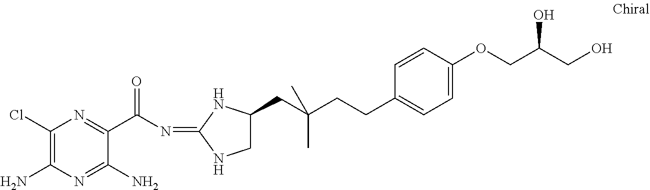 [SMILES: CC(C)(CCc1ccc(OC[C@@H](O)CO)cc1)C[C@H]1CN/C(=N/C(=O)c2nc(Cl)c(N)nc2N)N1] | 506 |
| 29 | 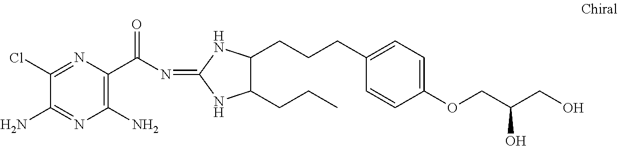 [SMILES: CCCC1N/C(=N/C(=O)c2nc(Cl)c(N)nc2N)NC1CCCc1ccc(OC[C@H](O)CO)cc1] | 506.37 |
| 30 | 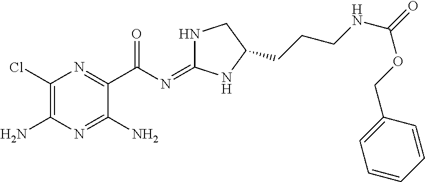 [SMILES: Nc1nc(N)c(C(=O)/N=C2\NC[C@H](CCCNC(=O)OCc3ccccc3)N2)nc1Cl] | 447.1 |
| 31 | 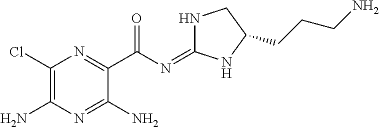 [SMILES: NCCC[C@H]1CN/C(=N\C(=O)c2nc(Cl)c(N)nc2N)N1] | 313.1 |
| 32 | 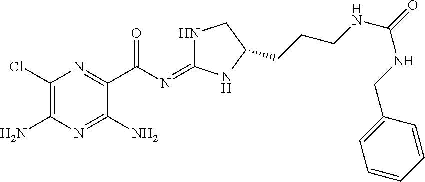 [SMILES: Nc1nc(N)c(C(=O)/N=C2\NC[C@H](CCCNC(=O)NCc3ccccc3)N2)nc1Cl] | 446.1 |
| 33 | 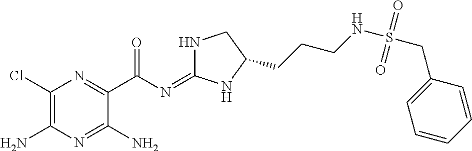 [SMILES: Nc1nc(N)c(C(=O)/N=C2\NC[C@H](CCCNS(=O)(=O)Cc3ccccc3)N2)nc1Cl] | 467.0 |
| 34 | 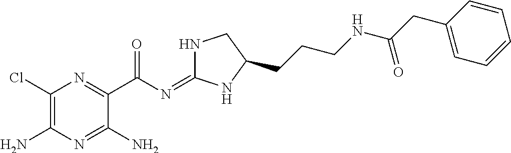 [SMILES: Nc1nc(N)c(C(=O)/N=C2\NC[C@@H](CCCNC(=O)Cc3ccccc3)N2)nc1Cl] | 430.98 |
| 35 | 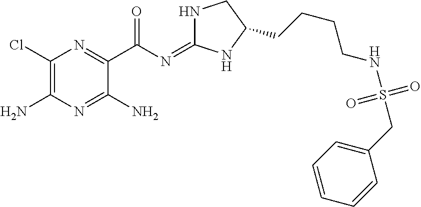 [SMILES: Nc1nc(N)c(C(=O)/N=C2\NC[C@H](CCCCNS(=O)(=O)Cc3ccccc3)N2)nc1Cl] | 481.0 |
| 36 | 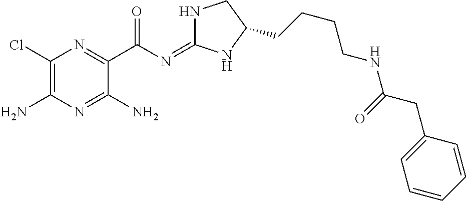 [SMILES: Nc1nc(N)c(C(=O)/N=C2\NC[C@H](CCCCNC(=O)Cc3ccccc3)N2)nc1Cl] | 445.1 |
| 37 | 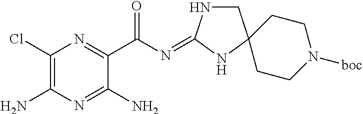 | 425 |
| 38 | 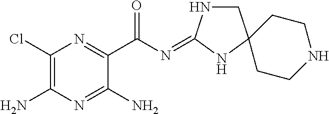 [SMILES: Nc1nc(N)c(C(=O)/N=C2\NCC3(CCNCC3)N2)nc1Cl] | 325 |
| 39 | 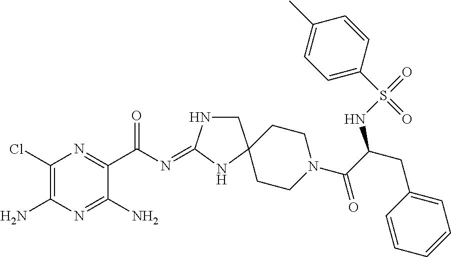 [SMILES: Cc1ccc(S(=O)(=O)N[C@@H](Cc2ccccc2)C(=O)N2CCC3(CC2)CN/C(=N\C(=O)c2nc(Cl)c(N)nc2N)N3)cc1] | 626.4 |
| 40 | 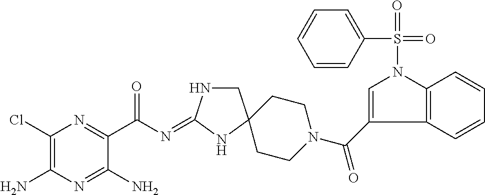 [SMILES: Nc1nc(N)c(C(=O)/N=C2\NCC3(CCN(C(=O)c4cn(S(=O)(=O)c5ccccc5)c5ccccc45)CC3)N2)nc1Cl] | 607.42 |
| 41 | 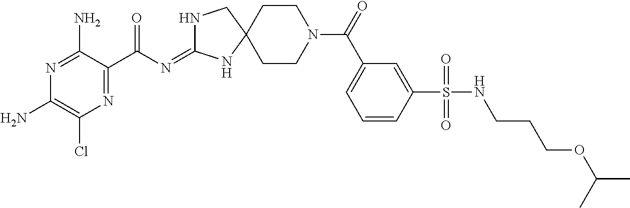 [SMILES: CC(C)OCCCNS(=O)(=O)c1cccc(C(=O)N2CCC3(CC2)CN/C(=N\C(=O)c2nc(Cl)c(N)nc2N)N3)c1] | 607.98 |
| 42 | 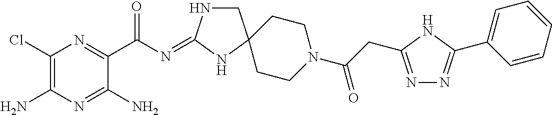 [SMILES: Nc1nc(N)c(C(=O)/N=C2\NCC3(CCN(C(=O)Cc4nnc(-c5ccccc5)[nH]4)CC3)N2)nc1Cl] | 510.4 |
| 43 | 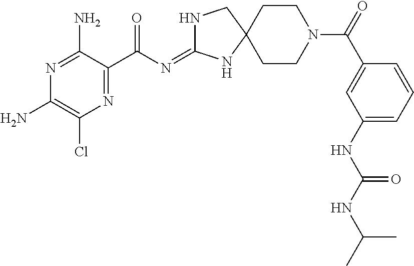 [SMILES: CC(C)NC(=O)Nc1cccc(C(=O)N2CCC3(CC2)CN/C(=N\C(=O)c2nc(Cl)c(N)nc2N)N3)c1] | 529.05 |
| 44 | 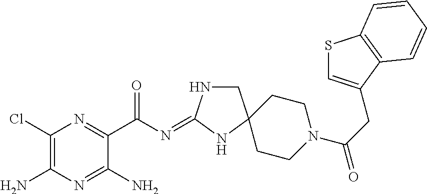 [SMILES: Nc1nc(N)c(C(=O)/N=C2\NCC3(CCN(C(=O)Cc4csc5ccccc45)CC3)N2)nc1Cl] | 499.0 |
| 45 | 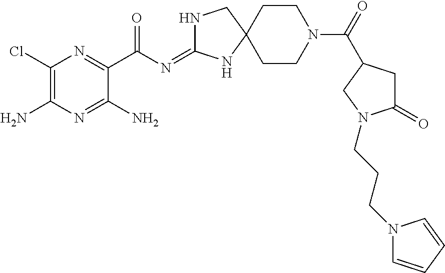 [SMILES: Nc1nc(N)c(C(=O)/N=C2\NCC3(CCN(C(=O)C4CC(=O)N(CCCn5cccc5)C4)CC3)N2)nc1Cl] | 542.91 |
| 46 | 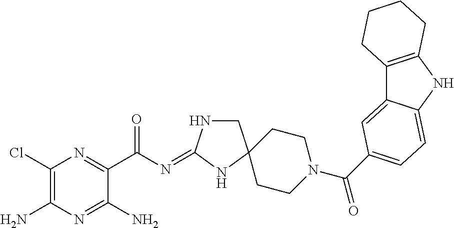 [SMILES: Nc1nc(N)c(C(=O)/N=C2\NCC3(CCN(C(=O)c4ccc5[nH]c6c(c5c4)CCCC6)CC3)N2)nc1Cl] | 552.1 |
| 47 | 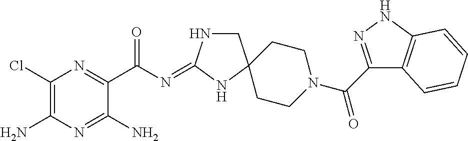 [SMILES: Nc1nc(N)c(C(=O)/N=C2\NCC3(CCN(C(=O)c4n[nH]c5ccccc45)CC3)N2)nc1Cl] | 469.17 |
| 48 | 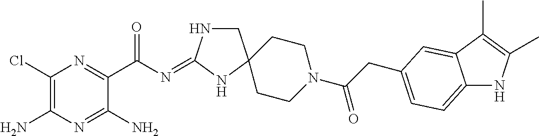 [SMILES: Cc1[nH]c2ccc(CC(=O)N3CCC4(CC3)CN/C(=N\C(=O)c3nc(Cl)c(N)nc3N)N4)cc2c1C] | 510.23 |
| 49 | 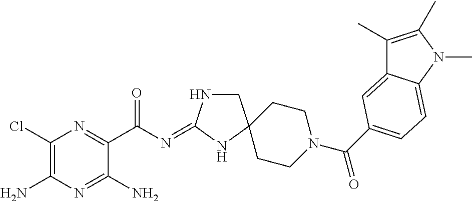 [SMILES: Cc1c(C)n(C)c2ccc(C(=O)N3CCC4(CC3)CN/C(=N\C(=O)c3nc(Cl)c(N)nc3N)N4)cc12] | 510.1 |
| 50 | 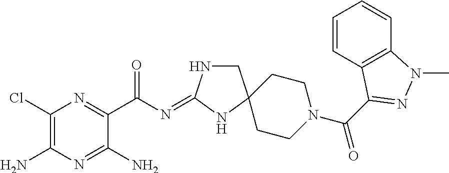 [SMILES: Cn1nc(C(=O)N2CCC3(CC2)CN/C(=N\C(=O)c2nc(Cl)c(N)nc2N)N3)c2ccccc21] | 483.1 |
| 51 | 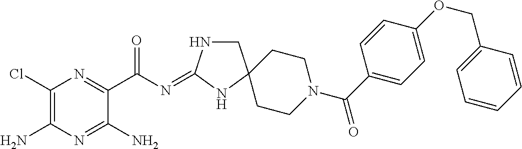 [SMILES: Nc1nc(N)c(C(=O)/N=C2\NCC3(CCN(C(=O)c4ccc(OCc5ccccc5)cc4)CC3)N2)nc1Cl] | 535.1 |
| 52 | 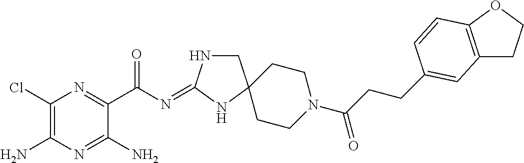 [SMILES: Nc1nc(N)c(C(=O)/N=C2\NCC3(CCN(C(=O)CCc4ccc5c(c4)CCO5)CC3)N2)nc1Cl] | 499.1 |
| 53 | 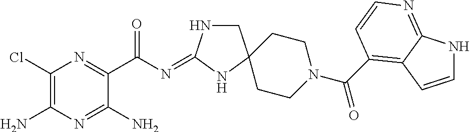 [SMILES: Nc1nc(N)c(C(=O)/N=C2\NCC3(CCN(C(=O)c4ccnc5[nH]ccc45)CC3)N2)nc1Cl] | 469.14 |
| 54 | 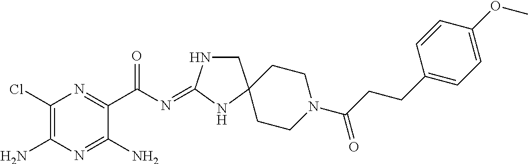 [SMILES: COc1ccc(CCC(=O)N2CCC3(CC2)CN/C(=N\C(=O)c2nc(Cl)c(N)nc2N)N3)cc1] | 487.0 |
| 55 | 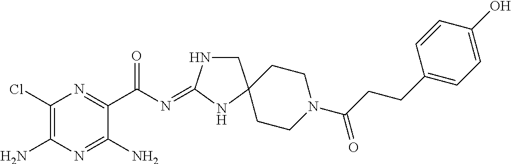 [SMILES: Nc1nc(N)c(C(=O)/N=C2\NCC3(CCN(C(=O)CCc4ccc(O)cc4)CC3)N2)nc1Cl] | 472.98 |
| 56 | 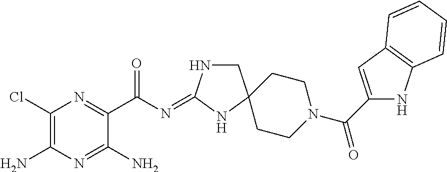 [SMILES: Nc1nc(N)c(C(=O)/N=C2\NCC3(CCN(C(=O)c4cc5ccccc5[nH]4)CC3)N2)nc1Cl] | 468.1 |
| 57 | 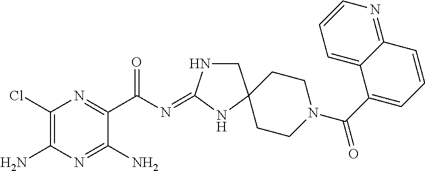 [SMILES: Nc1nc(N)c(C(=O)/N=C2\NCC3(CCN(C(=O)c4cccc5ncccc45)CC3)N2)nc1Cl] | 480.1 |
| 58 | 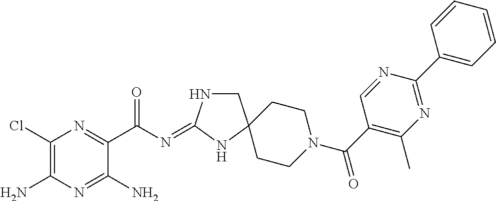 [SMILES: Cc1nc(-c2ccccc2)ncc1C(=O)N1CCC2(CC1)CN/C(=N\C(=O)c1nc(Cl)c(N)nc1N)N2] | 521.1 |
| 59 | 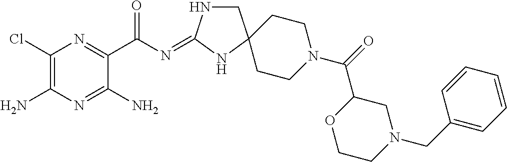 [SMILES: Nc1nc(N)c(C(=O)/N=C2\NCC3(CCN(C(=O)C4CN(Cc5ccccc5)CCO4)CC3)N2)nc1Cl] | 528.2 |
| 60 | 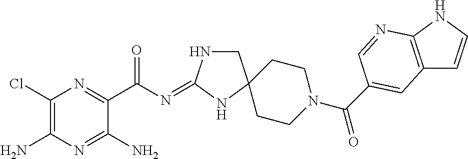 [SMILES: Nc1nc(N)c(C(=O)/N=C2\NCC3(CCN(C(=O)c4cnc5[nH]ccc5c4)CC3)N2)nc1Cl] | 469.08 |
| 61 | 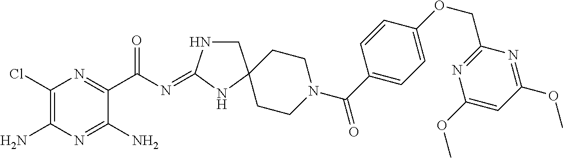 [SMILES: COc1cc(OC)nc(COc2ccc(C(=O)N3CCC4(CC3)CN/C(=N\C(=O)c3nc(Cl)c(N)nc3N)N4)cc2)n1] | 597.07 |
| 62 | 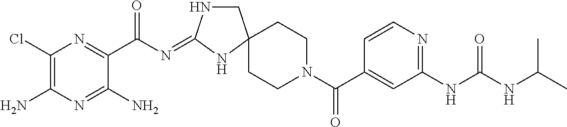 [SMILES: CC(C)NC(=O)Nc1cc(C(=O)N2CCC3(CC2)CN/C(=N\C(=O)c2nc(Cl)c(N)nc2N)N3)ccn1] | 530.21 |
| 63 | 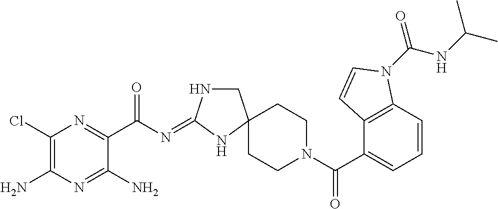 [SMILES: CC(C)NC(=O)n1ccc2c(C(=O)N3CCC4(CC3)CN/C(=N\C(=O)c3nc(Cl)c(N)nc3N)N4)cccc21] | 553.54 |
| 64 | 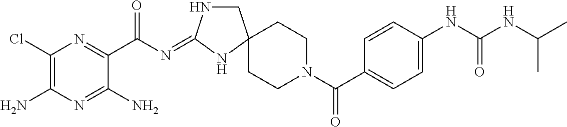 [SMILES: CC(C)NC(=O)Nc1ccc(C(=O)N2CCC3(CC2)CN/C(=N\C(=O)c2nc(Cl)c(N)nc2N)N3)cc1] | 529.54 |
| 65 | 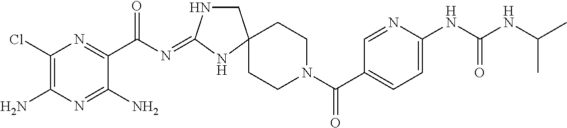 [SMILES: CC(C)NC(=O)Nc1ccc(C(=O)N2CCC3(CC2)CN/C(=N\C(=O)c2nc(Cl)c(N)nc2N)N3)cn1] | 530.46 |
| 66 | 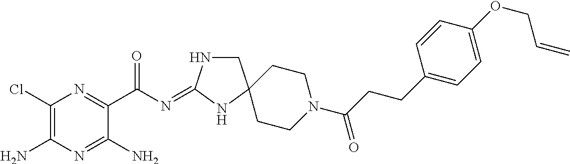 [SMILES: C=CCOc1ccc(CCC(=O)N2CCC3(CC2)CN/C(=N\C(=O)c2nc(Cl)c(N)nc2N)N3)cc1] | 513.40 |
| 67 | 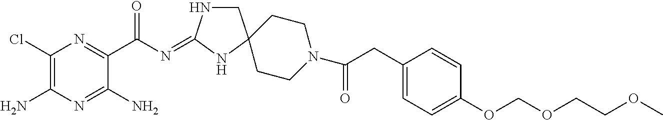 [SMILES: COCCOCOc1ccc(CC(=O)N2CCC3(CC2)CN/C(=N\C(=O)c2nc(Cl)c(N)nc2N)N3)cc1] | 547.42 |
| 68 | 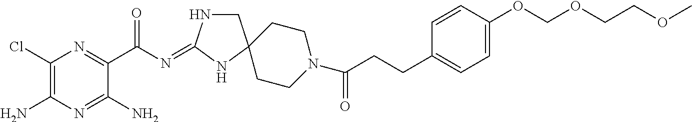 [SMILES: COCCOCOc1ccc(CCC(=O)N2CCC3(CC2)CN/C(=N\C(=O)c2nc(Cl)c(N)nc2N)N3)cc1] | 561.04 |
| 69 | 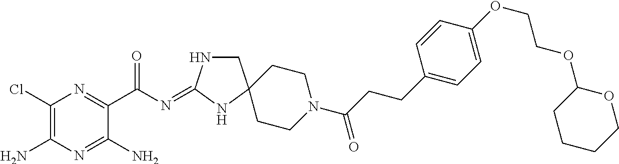 [SMILES: Nc1nc(N)c(C(=O)/N=C2\NCC3(CCN(C(=O)CCc4ccc(OCCOC5CCCCO5)cc4)CC3)N2)nc1Cl] | 601.10 |
| 70 | 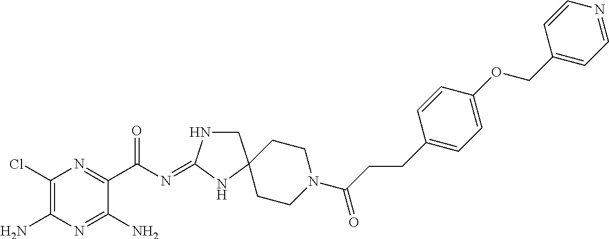 [SMILES: Nc1nc(N)c(C(=O)/N=C2\NCC3(CCN(C(=O)CCc4ccc(OCc5ccncc5)cc4)CC3)N2)nc1Cl] | 564.10 |
| 71 | 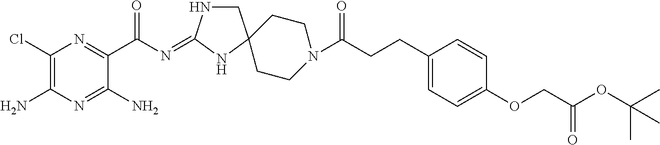 [SMILES: CC(C)(C)OC(=O)COc1ccc(CCC(=O)N2CCC3(CC2)CN/C(=N\C(=O)c2nc(Cl)c(N)nc2N)N3)cc1] | 587.50 |
| 72 | 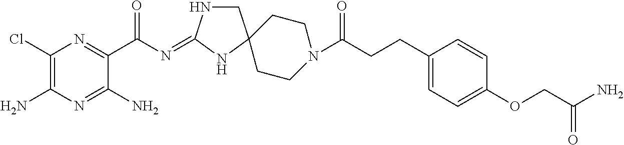 [SMILES: NC(=O)COc1ccc(CCC(=O)N2CCC3(CC2)CN/C(=N\C(=O)c2nc(Cl)c(N)nc2N)N3)cc1] | 530.10 |
| 73 | 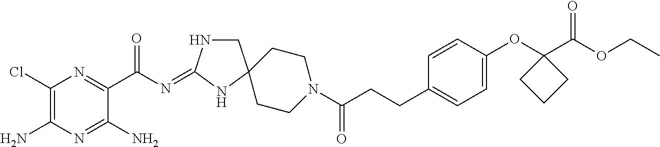 [SMILES: CCOC(=O)C1(Oc2ccc(CCC(=O)N3CCC4(CC3)CN/C(=N\C(=O)c3nc(Cl)c(N)nc3N)N4)cc2)CCC1] | 599.10 |
| 74 | 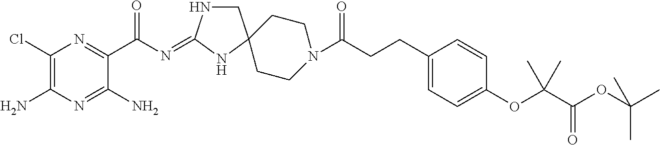 [SMILES: CC(C)(C)OC(=O)C(C)(C)Oc1ccc(CCC(=O)N2CCC3(CC2)CN/C(=N\C(=O)c2nc(Cl)c(N)nc2N)N3)cc1] | 615.20 |
| 75 | 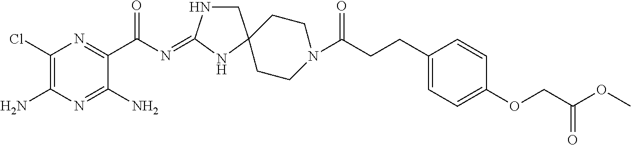 [SMILES: COC(=O)COc1ccc(CCC(=O)N2CCC3(CC2)CN/C(=N\C(=O)c2nc(Cl)c(N)nc2N)N3)cc1] | 545.10 |
| 76 | 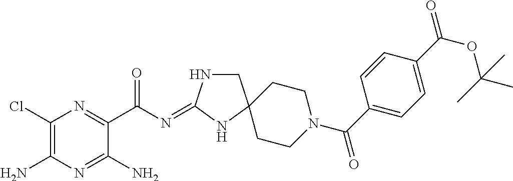 [SMILES: CC(C)(C)OC(=O)c1ccc(C(=O)N2CCC3(CC2)CN/C(=N\C(=O)c2nc(Cl)c(N)nc2N)N3)cc1] | 529.41 |
| 77 | 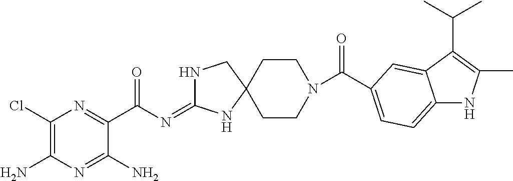 [SMILES: Cc1[nH]c2ccc(C(=O)N3CCC4(CC3)CN/C(=N\C(=O)c3nc(Cl)c(N)nc3N)N4)cc2c1C(C)C] | 524 |
| 78 | 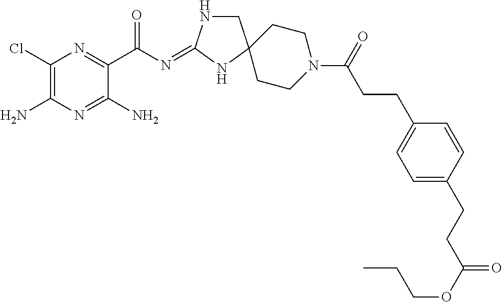 [SMILES: CCCOC(=O)CCc1ccc(CCC(=O)N2CCC3(CC2)CN/C(=N\C(=O)c2nc(Cl)c(N)nc2N)N3)cc1] | 571 |
| 79 | 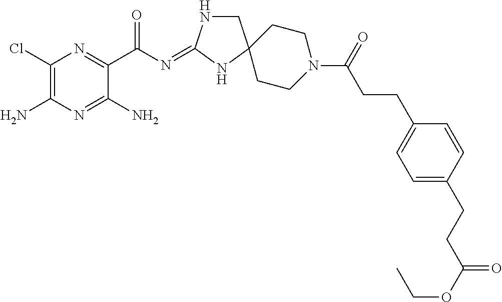 [SMILES: CCOC(=O)CCc1ccc(CCC(=O)N2CCC3(CC2)CN/C(=N\C(=O)c2nc(Cl)c(N)nc2N)N3)cc1] | 557 |
| 80 | 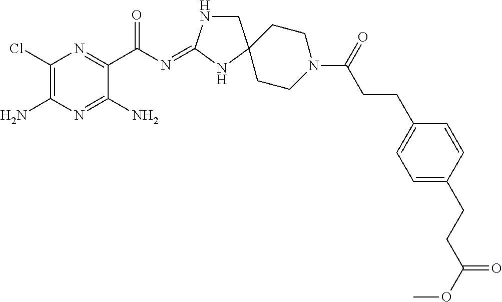 [SMILES: COC(=O)CCc1ccc(CCC(=O)N2CCC3(CC2)CN/C(=N\C(=O)c2nc(Cl)c(N)nc2N)N3)cc1] | 543 |
| 81 | 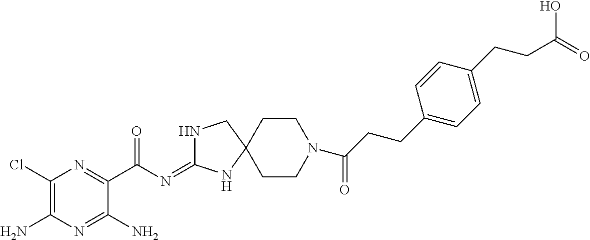 [SMILES: Nc1nc(N)c(C(=O)/N=C2\NCC3(CCN(C(=O)CCc4ccc(CCC(=O)O)cc4)CC3)N2)nc1Cl] | 529 |
| 82 | 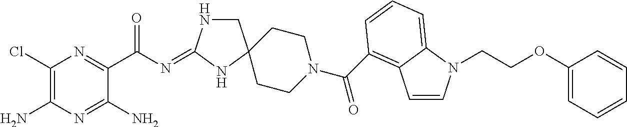 [SMILES: Nc1nc(N)c(C(=O)/N=C2\NCC3(CCN(C(=O)c4cccc5c4ccn5CCOc4ccccc4)CC3)N2)nc1Cl] | 588 |
| 83 | 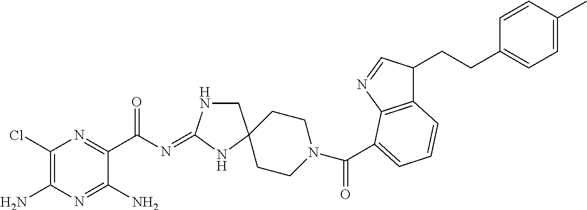 [SMILES: Cc1ccc(CCC2C=Nc3c(C(=O)N4CCC5(CC4)CN/C(=N\C(=O)c4nc(Cl)c(N)nc4N)N5)cccc32)cc1] | 586/588 |
| 84 | 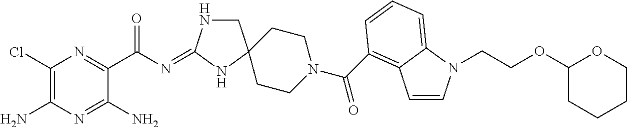 [SMILES: Nc1nc(N)c(C(=O)/N=C2\NCC3(CCN(C(=O)c4cccc5c4ccn5CCOC4CCCCO4)CC3)N2)nc1Cl] | 597 |
| 85 | 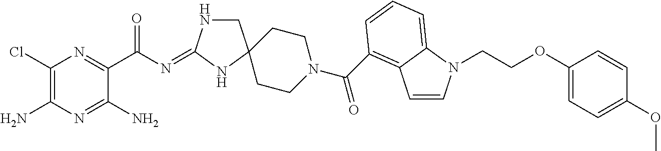 [SMILES: COc1ccc(OCCn2ccc3c(C(=O)N4CCC5(CC4)CN/C(=N\C(=O)c4nc(Cl)c(N)nc4N)N5)cccc32)cc1] | 618 |
| 86 | 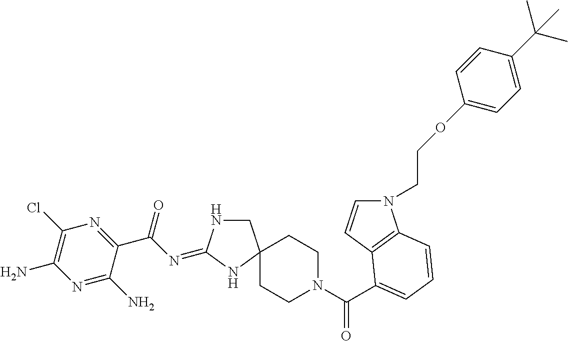 [SMILES: CC(C)(C)c1ccc(OCCn2ccc3c(C(=O)N4CCC5(CC4)CN/C(=N\C(=O)c4nc(Cl)c(N)nc4N)N5)cccc32)cc1] | 644/646 |
| 87 | 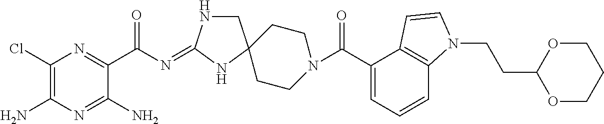 [SMILES: Nc1nc(N)c(C(=O)/N=C2\NCC3(CCN(C(=O)c4cccc5c4ccn5CCC4OCCCO4)CC3)N2)nc1Cl] | 582/584 |
| 88 | 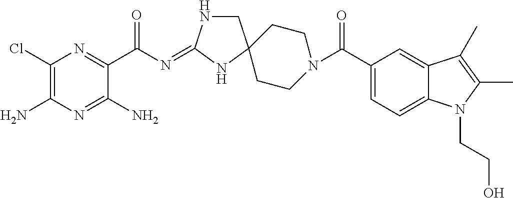 [SMILES: Cc1c(C)n(CCO)c2ccc(C(=O)N3CCC4(CC3)CN/C(=N\C(=O)c3nc(Cl)c(N)nc3N)N4)cc12] | 540 |
| 89 | 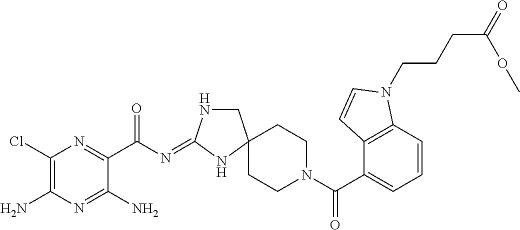 [SMILES: COC(=O)CCCn1ccc2c(C(=O)N3CCC4(CC3)CN/C(=N\C(=O)c3nc(Cl)c(N)nc3N)N4)cccc21] | 568/570 |
| 90 | 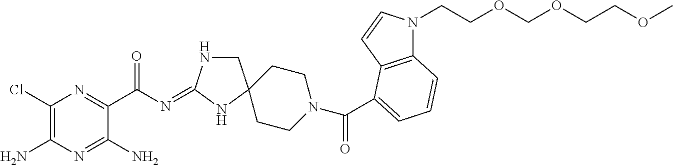 [SMILES: COCCOCOCCn1ccc2c(C(=O)N3CCC4(CC3)CN/C(=N\C(=O)c3nc(Cl)c(N)nc3N)N4)cccc21] | 600/602 |
| 91 | 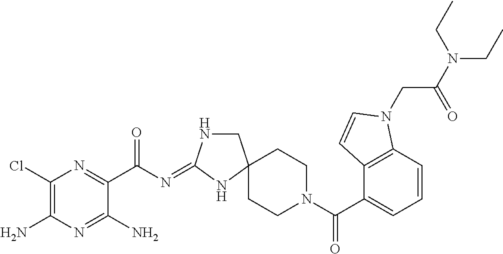 [SMILES: CCN(CC)C(=O)Cn1ccc2c(C(=O)N3CCC4(CC3)CN/C(=N\C(=O)c3nc(Cl)c(N)nc3N)N4)cccc21] | 581/583 |
| 92 | 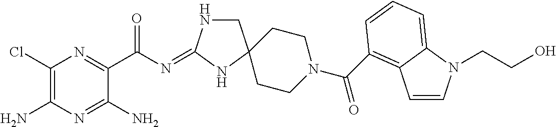 [SMILES: Nc1nc(N)c(C(=O)/N=C2\NCC3(CCN(C(=O)c4cccc5c4ccn5CCO)CC3)N2)nc1Cl] | 512/514 |
| 93 | 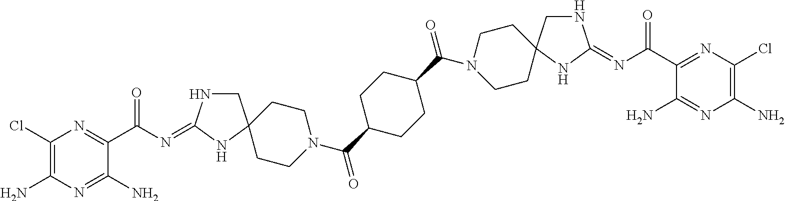 [SMILES: Nc1nc(N)c(C(=O)/N=C2\NCC3(CCN(C(=O)[C@H]4CC[C@@H](C(=O)N5CCC6(CC5)CN/C(=N\C(=O)c5nc(Cl)c(N)nc5N)N6)CC4)CC3)N2)nc1Cl] | 785 |
| 94 | 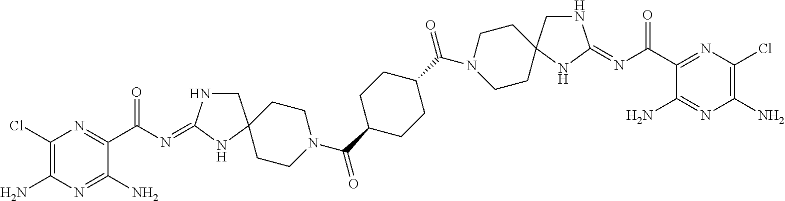 [SMILES: Nc1nc(N)c(C(=O)/N=C2\NCC3(CCN(C(=O)[C@H]4CC[C@H](C(=O)N5CCC6(CC5)CN/C(=N\C(=O)c5nc(Cl)c(N)nc5N)N6)CC4)CC3)N2)nc1Cl] | [M + 2H]²⁺ = 393 |
| 95 | 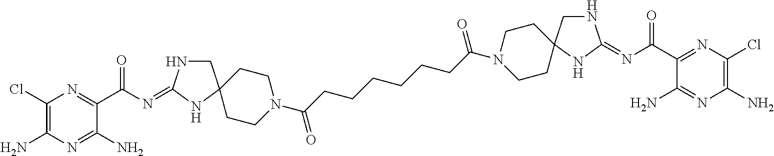 [SMILES: Nc1nc(N)c(C(=O)/N=C2\NCC3(CCN(C(=O)CCCCCCC(=O)N4CCC5(CC4)CN/C(=N\C(=O)c4nc(Cl)c(N)nc4N)N5)CC3)N2)nc1Cl] | 787 |
| 96 | 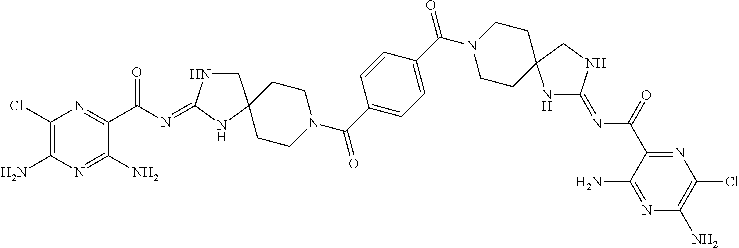 [SMILES: Nc1nc(N)c(C(=O)/N=C2\NCC3(CCN(C(=O)c4ccc(C(=O)N5CCC6(CC5)CN/C(=N\C(=O)c5nc(Cl)c(N)nc5N)N6)cc4)CC3)N2)nc1Cl] | 779 |
| 97 | 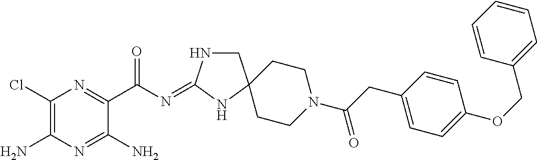 [SMILES: Nc1nc(N)c(C(=O)/N=C2\NCC3(CCN(C(=O)Cc4ccc(OCc5ccccc5)cc4)CC3)N2)nc1Cl] | 549 |
| 98 | 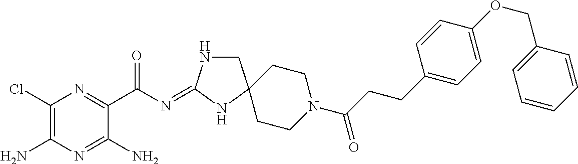 [SMILES: Nc1nc(N)c(C(=O)/N=C2\NCC3(CCN(C(=O)CCc4ccc(OCc5ccccc5)cc4)CC3)N2)nc1Cl] | 563 |
| 99 | 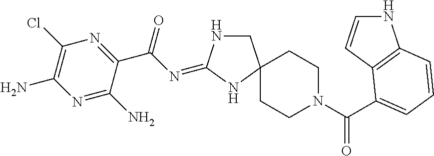 [SMILES: Nc1nc(N)c(C(=O)/N=C2\NCC3(CCN(C(=O)c4cccc5[nH]ccc45)CC3)N2)nc1Cl] | 468 |
| 100 | 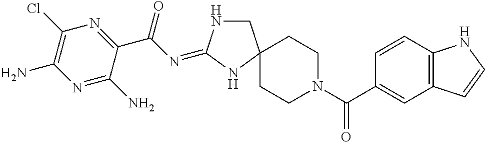 [SMILES: Nc1nc(N)c(C(=O)/N=C2\NCC3(CCN(C(=O)c4ccc5[nH]ccc5c4)CC3)N2)nc1Cl] | 468 |
| 101 | 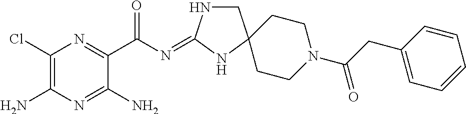 [SMILES: Nc1nc(N)c(C(=O)/N=C2\NCC3(CCN(C(=O)Cc4ccccc4)CC3)N2)nc1Cl] | 443 |
| 102 | 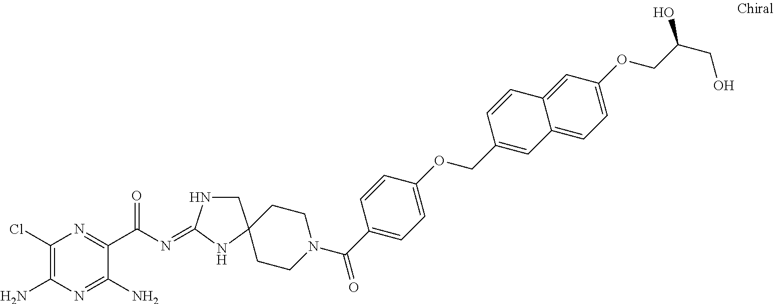 [SMILES: Nc1nc(N)c(C(=O)/N=C2\NCC3(CCN(C(=O)c4ccc(OCc5ccc6cc(OC[C@@H](O)CO)ccc6c5)cc4)CC3)N2)nc1Cl] | 675 |
| 103 | 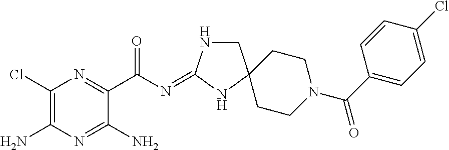 [SMILES: Nc1nc(N)c(C(=O)/N=C2\NCC3(CCN(C(=O)c4ccc(Cl)cc4)CC3)N2)nc1Cl] | 463 |
| 104 | 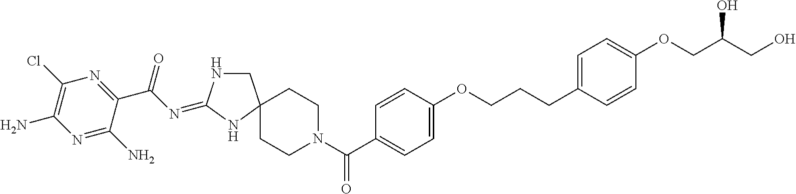 [SMILES: Nc1nc(N)c(C(=O)/N=C2\NCC3(CCN(C(=O)c4ccc(OCCCc5ccc(OC[C@@H](O)CO)cc5)cc4)CC3)N2)nc1Cl] | 653 |
| 105 | 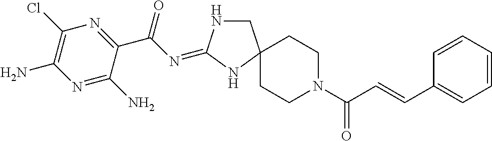 [SMILES: Nc1nc(N)c(C(=O)/N=C2\NCC3(CCN(C(=O)/C=C/c4ccccc4)CC3)N2)nc1Cl] | 455 |
| 106 | 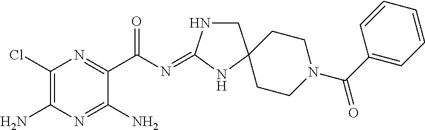 [SMILES: Nc1nc(N)c(C(=O)/N=C2\NCC3(CCN(C(=O)c4ccccc4)CC3)N2)nc1Cl] | 429 |
| 107 | 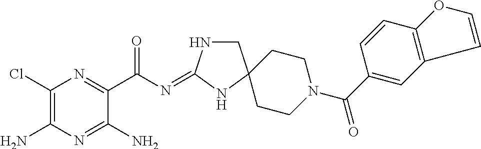 [SMILES: Nc1nc(N)c(C(=O)/N=C2\NCC3(CCN(C(=O)c4ccc5occc5c4)CC3)N2)nc1Cl] | 469 |
| 108 | 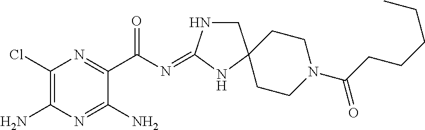 [SMILES: CCCCCC(=O)N1CCC2(CC1)CN/C(=N\C(=O)c1nc(Cl)c(N)nc1N)N2] | 423 |
| 110 | 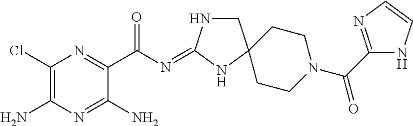 [SMILES: Nc1nc(N)c(C(=O)/N=C2\NCC3(CCN(C(=O)c4ncc[nH]4)CC3)N2)nc1Cl] | 419 |
| 111 | 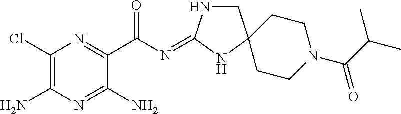 [SMILES: CC(C)C(=O)N1CCC2(CC1)CN/C(=N\C(=O)c1nc(Cl)c(N)nc1N)N2] | 395 |
| 112 | 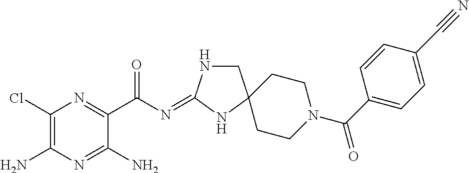 [SMILES: N#Cc1ccc(C(=O)N2CCC3(CC2)CN/C(=N\C(=O)c2nc(Cl)c(N)nc2N)N3)cc1] | 454 |
| 113 | 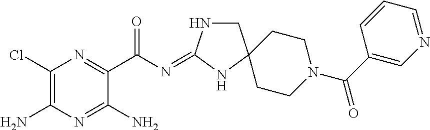 [SMILES: Nc1nc(N)c(C(=O)/N=C2\NCC3(CCN(C(=O)c4cccnc4)CC3)N2)nc1Cl] | 430 |
| 114 | 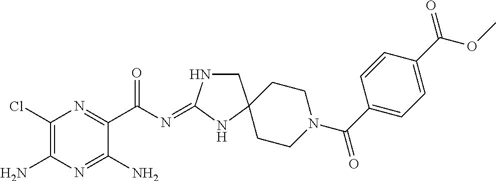 [SMILES: COC(=O)c1ccc(C(=O)N2CCC3(CC2)CN/C(=N\C(=O)c2nc(Cl)c(N)nc2N)N3)cc1] | 487 |
| 115 | 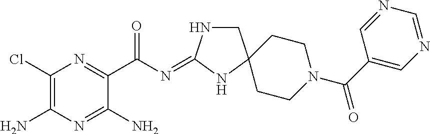 [SMILES: Nc1nc(N)c(C(=O)/N=C2\NCC3(CCN(C(=O)c4cncnc4)CC3)N2)nc1Cl] | 431 |
| 116 | 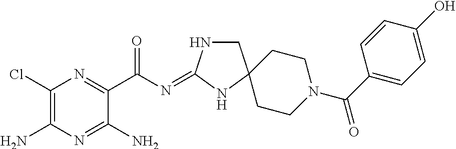 [SMILES: Nc1nc(N)c(C(=O)/N=C2\NCC3(CCN(C(=O)c4ccc(O)cc4)CC3)N2)nc1Cl] | 445 |
| 117 | 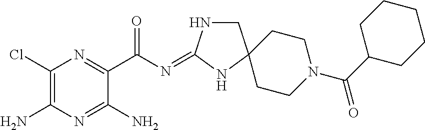 [SMILES: Nc1nc(N)c(C(=O)/N=C2\NCC3(CCN(C(=O)C4CCCCC4)CC3)N2)nc1Cl] | 435 |
| 118 | 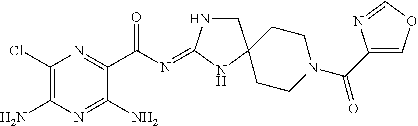 [SMILES: Nc1nc(N)c(C(=O)/N=C2\NCC3(CCN(C(=O)c4cocn4)CC3)N2)nc1Cl] | 420 |
| 119 | 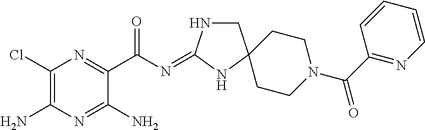 [SMILES: Nc1nc(N)c(C(=O)/N=C2\NCC3(CCN(C(=O)c4ccccn4)CC3)N2)nc1Cl] | 430 |
| 120 | 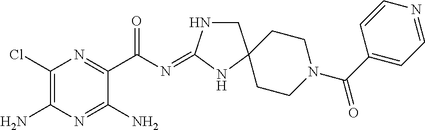 [SMILES: Nc1nc(N)c(C(=O)/N=C2\NCC3(CCN(C(=O)c4ccncc4)CC3)N2)nc1Cl] | 430 |
| 121 | 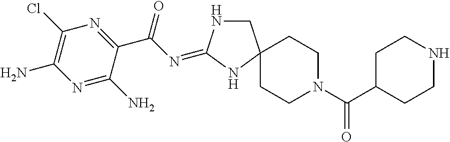 [SMILES: Nc1nc(N)c(C(=O)/N=C2\NCC3(CCN(C(=O)C4CCNCC4)CC3)N2)nc1Cl] | 436 |
| 122 | 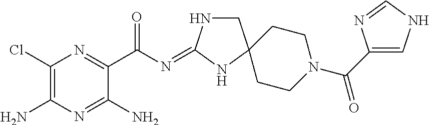 [SMILES: Nc1nc(N)c(C(=O)/N=C2\NCC3(CCN(C(=O)c4c[nH]cn4)CC3)N2)nc1Cl] | 419 |
| 123 | 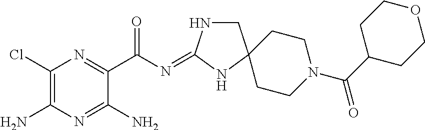 [SMILES: Nc1nc(N)c(C(=O)/N=C2\NCC3(CCN(C(=O)C4CCOCC4)CC3)N2)nc1Cl] | 437 |
| 124 | 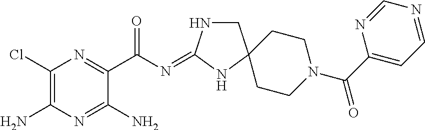 [SMILES: Nc1nc(N)c(C(=O)/N=C2\NCC3(CCN(C(=O)c4ccncn4)CC3)N2)nc1Cl] | 431 |
| 125 | 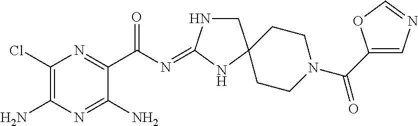 [SMILES: Nc1nc(N)c(C(=O)/N=C2\NCC3(CCN(C(=O)c4cnco4)CC3)N2)nc1Cl] | 420 |
| 126 | 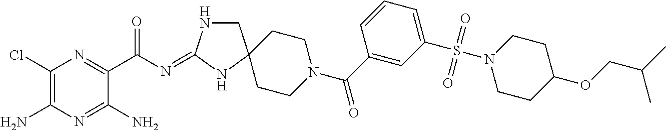 [SMILES: CC(C)COC1CCN(S(=O)(=O)c2cccc(C(=O)N3CCC4(CC3)CN/C(=N\C(=O)c3nc(Cl)c(N)nc3N)N4)c2)CC1] | 648.4 |
| 127 | 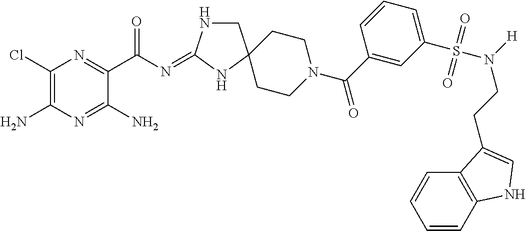 [SMILES: Nc1nc(N)c(C(=O)/N=C2\NCC3(CCN(C(=O)c4cccc(S(=O)(=O)NCCc5c[nH]c6ccccc56)c4)CC3)N2)nc1Cl] | 651.3 |
| 128 | 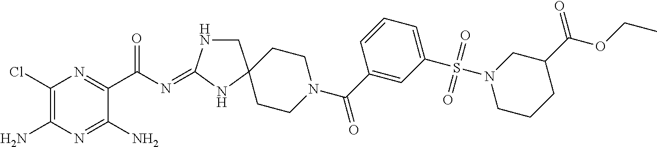 [SMILES: CCOC(=O)C1CCCN(S(=O)(=O)c2cccc(C(=O)N3CCC4(CC3)CN/C(=N\C(=O)c3nc(Cl)c(N)nc3N)N4)c2)C1] | 648.3 |
| 129 | 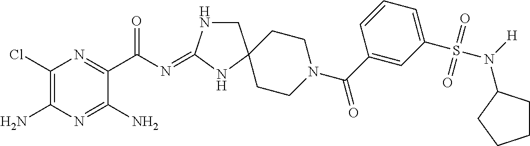 [SMILES: Nc1nc(N)c(C(=O)/N=C2\NCC3(CCN(C(=O)c4cccc(S(=O)(=O)NC5CCCC5)c4)CC3)N2)nc1Cl] | 576.3 |
| 130 | 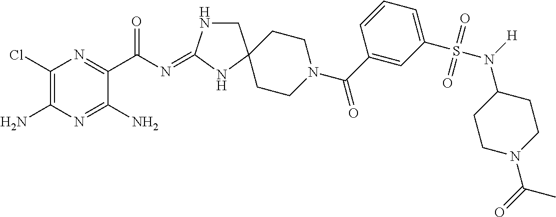 [SMILES: CC(=O)N1CCC(NS(=O)(=O)c2cccc(C(=O)N3CCC4(CC3)CN/C(=N\C(=O)c3nc(Cl)c(N)nc3N)N4)c2)CC1] | 633.3 |
| 131 | 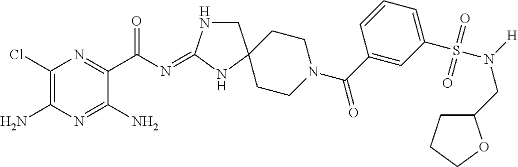 [SMILES: Nc1nc(N)c(C(=O)/N=C2\NCC3(CCN(C(=O)c4cccc(S(=O)(=O)NCC5CCCO5)c4)CC3)N2)nc1Cl] | 592.3 |
| 132 | 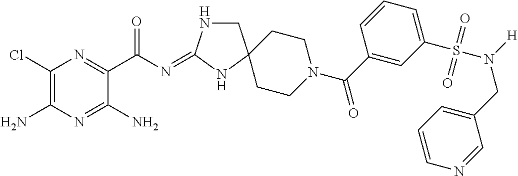 [SMILES: Nc1nc(N)c(C(=O)/N=C2\NCC3(CCN(C(=O)c4cccc(S(=O)(=O)NCc5cccnc5)c4)CC3)N2)nc1Cl] | 599.3 |
| 133 | 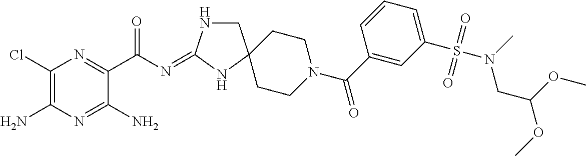 [SMILES: COC(CN(C)S(=O)(=O)c1cccc(C(=O)N2CCC3(CC2)CN/C(=N\C(=O)c2nc(Cl)c(N)nc2N)N3)c1)OC] | 610.3 |
| 134 | 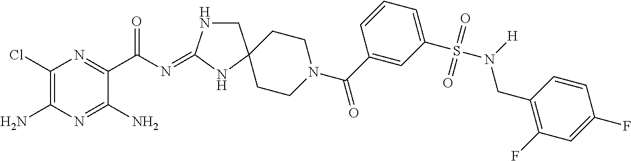 [SMILES: Nc1nc(N)c(C(=O)/N=C2\NCC3(CCN(C(=O)c4cccc(S(=O)(=O)NCc5ccc(F)cc5F)c4)CC3)N2)nc1Cl] | 634.3 |
| 135 | 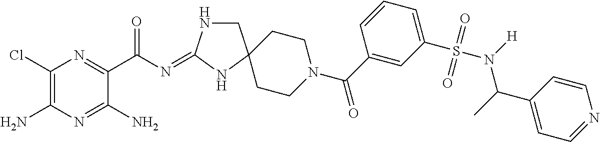 [SMILES: CC(NS(=O)(=O)c1cccc(C(=O)N2CCC3(CC2)CN/C(=N\C(=O)c2nc(Cl)c(N)nc2N)N3)c1)c1ccncc1] | 613.3 |
| 136 | 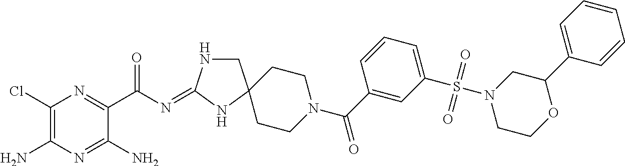 [SMILES: Nc1nc(N)c(C(=O)/N=C2\NCC3(CCN(C(=O)c4cccc(S(=O)(=O)N5CCOC(c6ccccc6)C5)c4)CC3)N2)nc1Cl] | 654.3 |
| 137 | 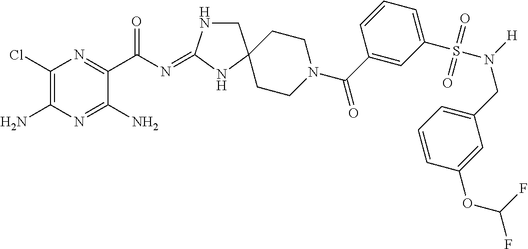 [SMILES: Nc1nc(N)c(C(=O)/N=C2\NCC3(CCN(C(=O)c4cccc(S(=O)(=O)NCc5cccc(OC(F)F)c5)c4)CC3)N2)nc1Cl] | 664.3 |
| 138 | 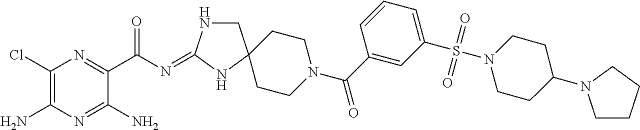 [SMILES: Nc1nc(N)c(C(=O)/N=C2\NCC3(CCN(C(=O)c4cccc(S(=O)(=O)N5CCC(N6CCCC6)CC5)c4)CC3)N2)nc1Cl] | 645.4 |
| 139 | 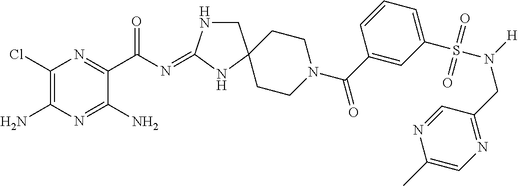 [SMILES: Cc1cnc(CNS(=O)(=O)c2cccc(C(=O)N3CCC4(CC3)CN/C(=N\C(=O)c3nc(Cl)c(N)nc3N)N4)c2)cn1] | 614.3 |
| 140 | 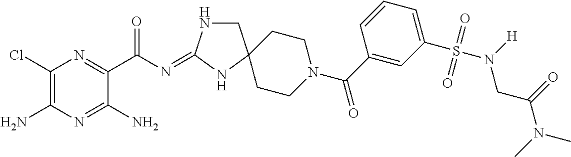 [SMILES: CN(C)C(=O)CNS(=O)(=O)c1cccc(C(=O)N2CCC3(CC2)CN/C(=N\C(=O)c2nc(Cl)c(N)nc2N)N3)c1] | 593.4 |
| 141 | 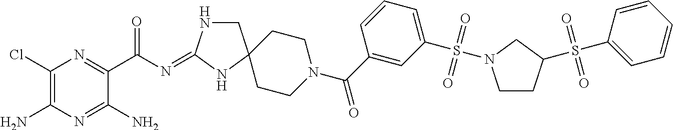 [SMILES: Nc1nc(N)c(C(=O)/N=C2\NCC3(CCN(C(=O)c4cccc(S(=O)(=O)N5CCC(S(=O)(=O)c6ccccc6)C5)c4)CC3)N2)nc1Cl] | 702.3 |
| 142 | 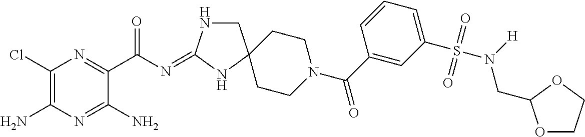 [SMILES: Nc1nc(N)c(C(=O)/N=C2\NCC3(CCN(C(=O)c4cccc(S(=O)(=O)NCC5OCCO5)c4)CC3)N2)nc1Cl] | 594.3 |
| 143 | 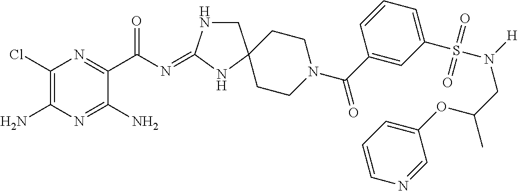 [SMILES: CC(CNS(=O)(=O)c1cccc(C(=O)N2CCC3(CC2)CN/C(=N\C(=O)c2nc(Cl)c(N)nc2N)N3)c1)Oc1cccnc1] | 643.3 |
| 144 | 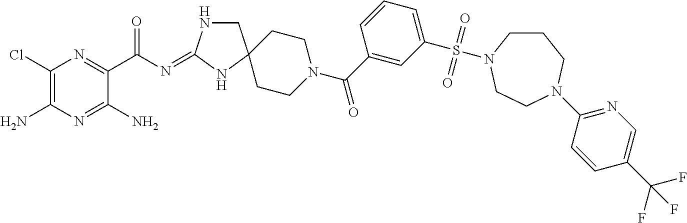 [SMILES: Nc1nc(N)c(C(=O)/N=C2\NCC3(CCN(C(=O)c4cccc(S(=O)(=O)N5CCCN(c6ccc(C(F)(F)F)cn6)CC5)c4)CC3)N2)nc1Cl] | 736.4 |
| 145 | 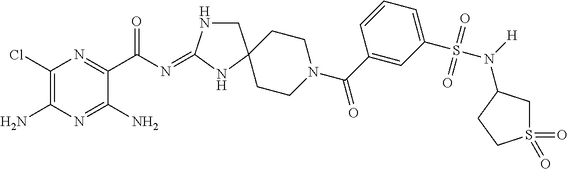 [SMILES: Nc1nc(N)c(C(=O)/N=C2\NCC3(CCN(C(=O)c4cccc(S(=O)(=O)NC5CCS(=O)(=O)C5)c4)CC3)N2)nc1Cl] | 626.3 |
| 146 | 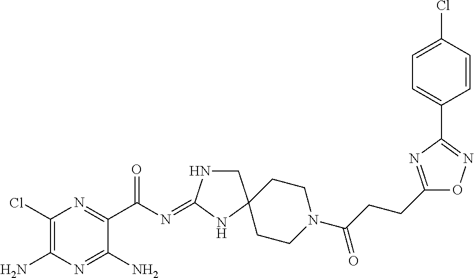 [SMILES: Nc1nc(N)c(C(=O)/N=C2\NCC3(CCN(C(=O)CCc4nc(-c5ccc(Cl)cc5)no4)CC3)N2)nc1Cl] | 559.3 |
| 147 | 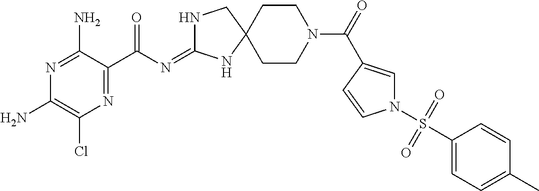 [SMILES: Cc1ccc(S(=O)(=O)n2ccc(C(=O)N3CCC4(CC3)CN/C(=N\C(=O)c3nc(Cl)c(N)nc3N)N4)c2)cc1] | 572.08 |
| 148 | 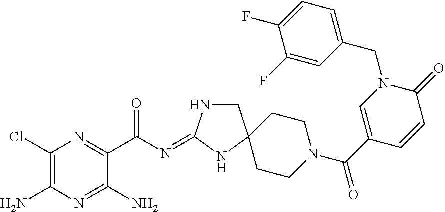 [SMILES: Nc1nc(N)c(C(=O)/N=C2\NCC3(CCN(C(=O)c4ccc(=O)n(Cc5ccc(F)c(F)c5)c4)CC3)N2)nc1Cl] | 572.0 |
| 149 | 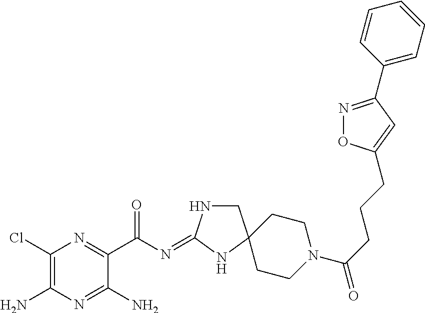 [SMILES: Nc1nc(N)c(C(=O)/N=C2\NCC3(CCN(C(=O)CCCc4cc(-c5ccccc5)no4)CC3)N2)nc1Cl] | 538.4 |
| 150 | 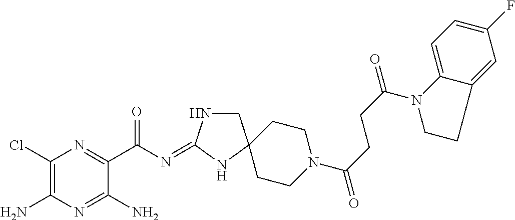 [SMILES: Nc1nc(N)c(C(=O)/N=C2\NCC3(CCN(C(=O)CCC(=O)N4CCc5cc(F)ccc54)CC3)N2)nc1Cl] | 544.4 |
| 151 | 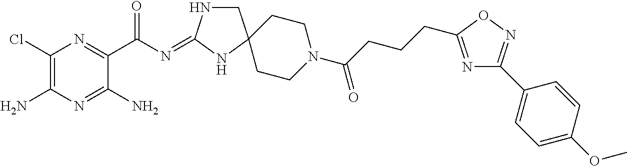 [SMILES: COc1ccc(-c2noc(CCCC(=O)N3CCC4(CC3)CN/C(=N\C(=O)c3nc(Cl)c(N)nc3N)N4)n2)cc1] | 569.4 |
| 152 | 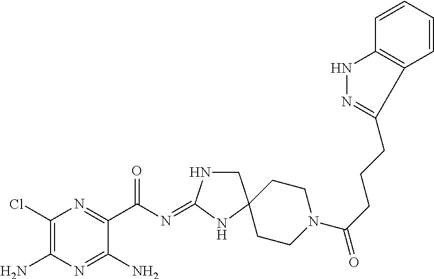 [SMILES: Nc1nc(N)c(C(=O)/N=C2\NCC3(CCN(C(=O)CCCc4n[nH]c5ccccc45)CC3)N2)nc1Cl] | 511.4 |
| 153 | 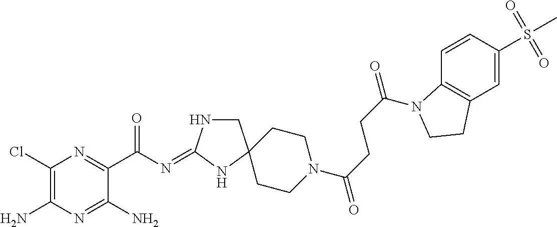 [SMILES: CS(=O)(=O)c1ccc2c(c1)CCN2C(=O)CCC(=O)N1CCC2(CC1)CN/C(=N\C(=O)c1nc(Cl)c(N)nc1N)N2] | 604.3 |
| 154 | 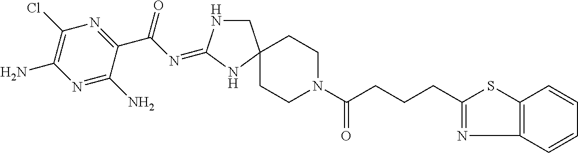 [SMILES: Nc1nc(N)c(C(=O)/N=C2\NCC3(CCN(C(=O)CCCc4nc5ccccc5s4)CC3)N2)nc1Cl] | 528.3 |
| 155 | 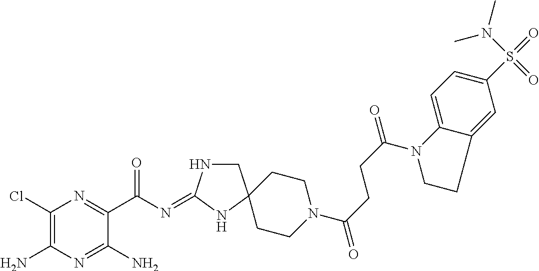 [SMILES: CN(C)S(=O)(=O)c1ccc2c(c1)CCN2C(=O)CCC(=O)N1CCC2(CC1)CN/C(=N\C(=O)c1nc(Cl)c(N)nc1N)N2] | 633.4 |
| 156 | 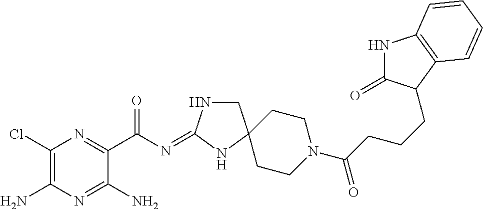 [SMILES: Nc1nc(N)c(C(=O)/N=C2\NCC3(CCN(C(=O)CCCC4C(=O)Nc5ccccc54)CC3)N2)nc1Cl] | 526.3 |
| 157 | 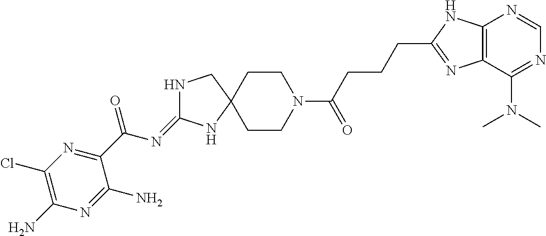 [SMILES: CN(C)c1ncnc2[nH]c(CCCC(=O)N3CCC4(CC3)CN/C(=N\C(=O)c3nc(Cl)c(N)nc3N)N4)nc12] | 556.4 |
| 158 | 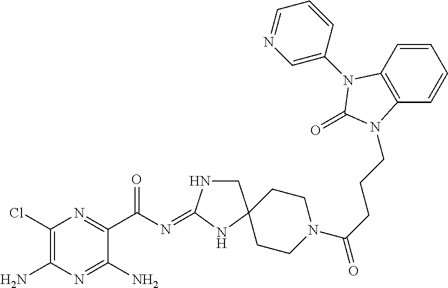 [SMILES: Nc1nc(N)c(C(=O)/N=C2\NCC3(CCN(C(=O)CCCn4c(=O)n(-c5cccnc5)c5ccccc54)CC3)N2)nc1Cl] | 604.4 |
| 159 | 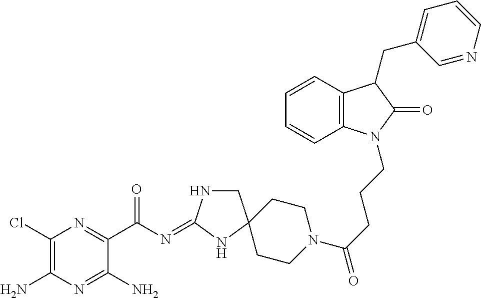 [SMILES: Nc1nc(N)c(C(=O)/N=C2\NCC3(CCN(C(=O)CCCN4C(=O)C(Cc5cccnc5)c5ccccc54)CC3)N2)nc1Cl] | 617.4 |
| 160 | 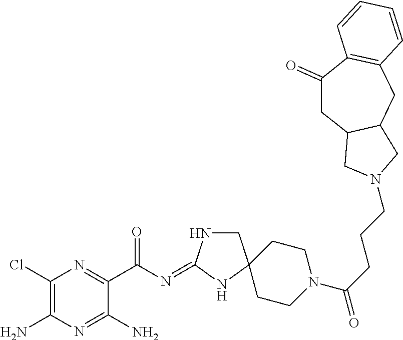 [SMILES: Nc1nc(N)c(C(=O)/N=C2\NCC3(CCN(C(=O)CCCN4CC5CC(=O)c6ccccc6CC5C4)CC3)N2)nc1Cl] | 594.4 |
| 161 | 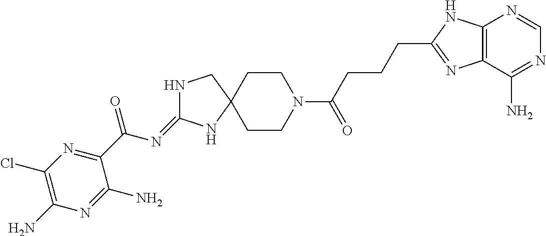 [SMILES: Nc1nc(N)c(C(=O)/N=C2\NCC3(CCN(C(=O)CCCc4nc5c(N)ncnc5[nH]4)CC3)N2)nc1Cl] | 528.4 |
| 162 | 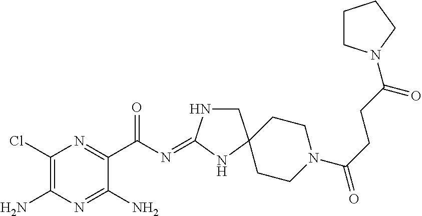 [SMILES: Nc1nc(N)c(C(=O)/N=C2\NCC3(CCN(C(=O)CCC(=O)N4CCCC4)CC3)N2)nc1Cl] | 478.3 |
| 163 | 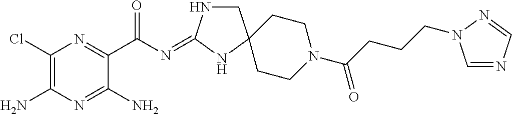 [SMILES: Nc1nc(N)c(C(=O)/N=C2\NCC3(CCN(C(=O)CCCn4cncn4)CC3)N2)nc1Cl] | 462.3 |
| 164 | 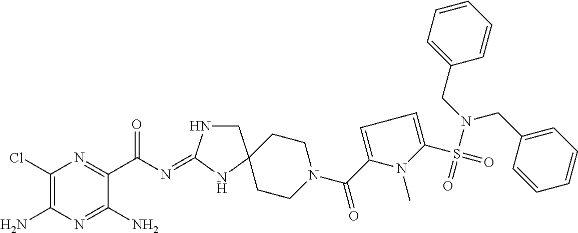 [SMILES: Cn1c(C(=O)N2CCC3(CC2)CN/C(=N\C(=O)c2nc(Cl)c(N)nc2N)N3)ccc1S(=O)(=O)N(Cc1ccccc1)Cc1ccccc1] | 691.04 |
| 165 | 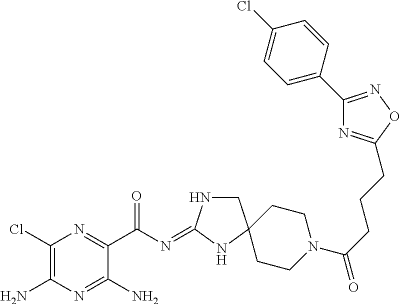 [SMILES: Nc1nc(N)c(C(=O)/N=C2\NCC3(CCN(C(=O)CCCc4nc(-c5ccc(Cl)cc5)no4)CC3)N2)nc1Cl] | 573.05 |
| 166 | 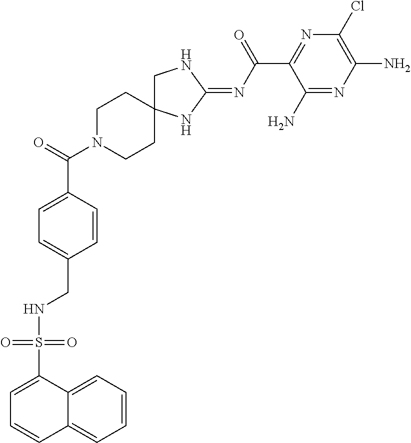 [SMILES: Nc1nc(N)c(C(=O)/N=C2\NCC3(CCN(C(=O)c4ccc(CNS(=O)(=O)c5cccc6ccccc56)cc4)CC3)N2)nc1Cl] | 648.06 |
| 167 | 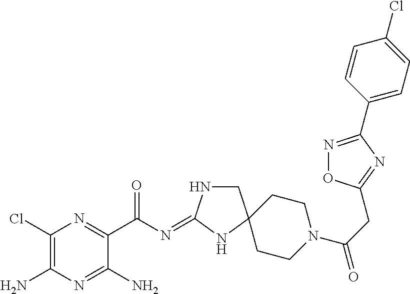 [SMILES: Nc1nc(N)c(C(=O)/N=C2\NCC3(CCN(C(=O)Cc4nc(-c5ccc(Cl)cc5)no4)CC3)N2)nc1Cl] | 545.3 |
| 168 | 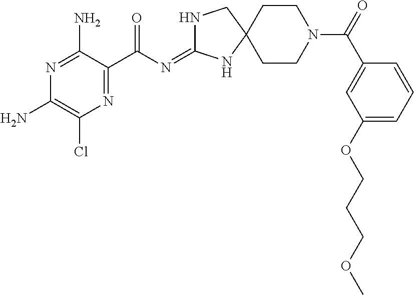 [SMILES: COCCCOc1cccc(C(=O)N2CCC3(CC2)CN/C(=N\C(=O)c2nc(Cl)c(N)nc2N)N3)c1] | 517.07 |
| 169 | 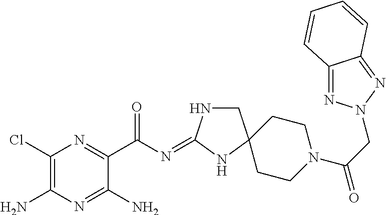 [SMILES: Nc1nc(N)c(C(=O)/N=C2\NCC3(CCN(C(=O)Cn4nc5ccccc5n4)CC3)N2)nc1Cl] | 484.04 |
| 170 | 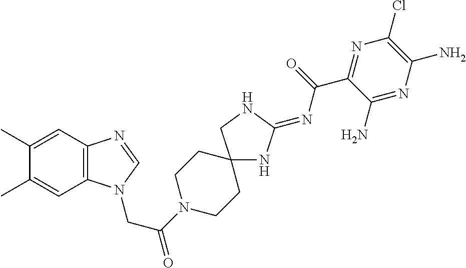 [SMILES: Cc1cc2ncn(CC(=O)N3CCC4(CC3)CN/C(=N\C(=O)c3nc(Cl)c(N)nc3N)N4)c2cc1C] | 511.04 |
| 171 | 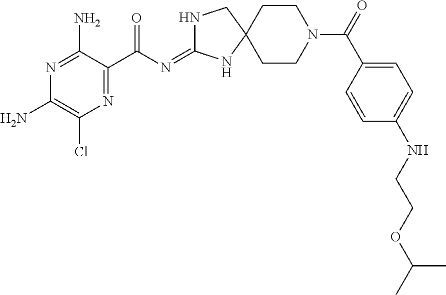 [SMILES: CC(C)OCCNc1ccc(C(=O)N2CCC3(CC2)CN/C(=N\C(=O)c2nc(Cl)c(N)nc2N)N3)cc1] | 530.08 |
| 172 | 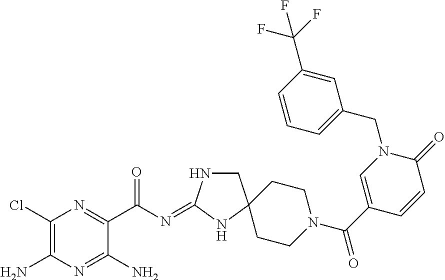 [SMILES: Nc1nc(N)c(C(=O)/N=C2\NCC3(CCN(C(=O)c4ccc(=O)n(Cc5cccc(C(F)(F)F)c5)c4)CC3)N2)nc1Cl] | 603.99 |
| 173 | 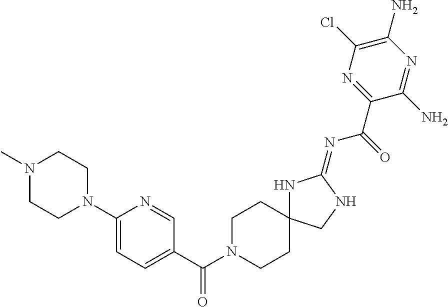 [SMILES: CN1CCN(c2ccc(C(=O)N3CCC4(CC3)CN/C(=N\C(=O)c3nc(Cl)c(N)nc3N)N4)cn2)CC1] | 530.19 |
| 174 | 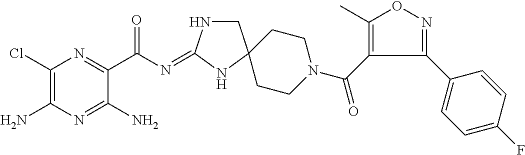 [SMILES: Cc1onc(-c2ccc(F)cc2)c1C(=O)N1CCC2(CC1)CN/C(=N\C(=O)c1nc(Cl)c(N)nc1N)N2] | 527.99 |
| 175 | 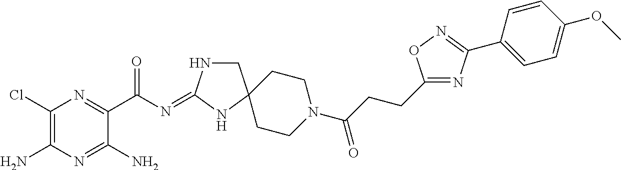 [SMILES: COc1ccc(-c2noc(CCC(=O)N3CCC4(CC3)CN/C(=N\C(=O)c3nc(Cl)c(N)nc3N)N4)n2)cc1] | 555.07 |
| 176 | 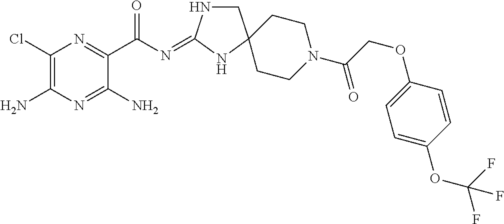 [SMILES: Nc1nc(N)c(C(=O)/N=C2\NCC3(CCN(C(=O)COc4ccc(OC(F)(F)F)cc4)CC3)N2)nc1Cl] | 608.05 |
| 177 | 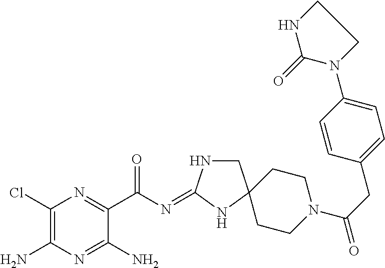 [SMILES: Nc1nc(N)c(C(=O)/N=C2\NCC3(CCN(C(=O)Cc4ccc(N5CCNC5=O)cc4)CC3)N2)nc1Cl] | 527.07 |
| 178 | 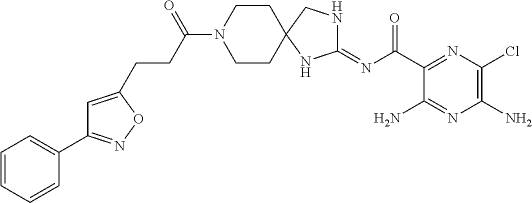 [SMILES: Nc1nc(N)c(C(=O)/N=C2\NCC3(CCN(C(=O)CCc4cc(-c5ccccc5)no4)CC3)N2)nc1Cl] | 524.1 |
| 179 | 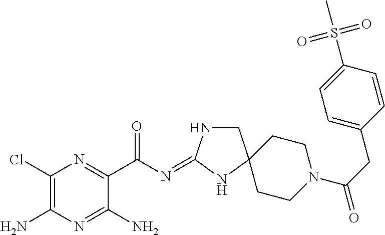 [SMILES: CS(=O)(=O)c1ccc(CC(=O)N2CCC3(CC2)CN/C(=N\C(=O)c2nc(Cl)c(N)nc2N)N3)cc1] | 520.99 |
| 180 | 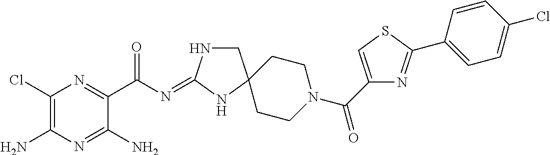 [SMILES: Nc1nc(N)c(C(=O)/N=C2\NCC3(CCN(C(=O)c4csc(-c5ccc(Cl)cc5)n4)CC3)N2)nc1Cl] | 545.95 |
| 181 | 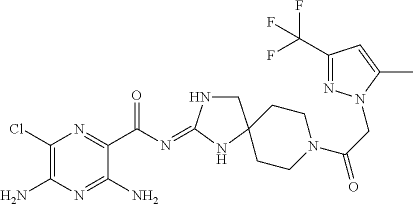 [SMILES: Cc1cc(C(F)(F)F)nn1CC(=O)N1CCC2(CC1)CN/C(=N\C(=O)c1nc(Cl)c(N)nc1N)N2] | 514.98 |
| 182 | 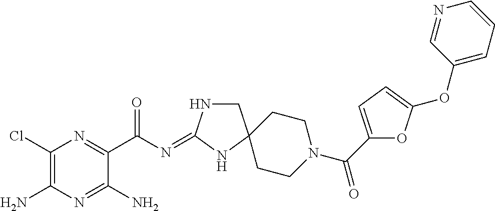 [SMILES: Nc1nc(N)c(C(=O)/N=C2\NCC3(CCN(C(=O)c4ccc(Oc5cccnc5)o4)CC3)N2)nc1Cl] | 512.01 |
| 183 | 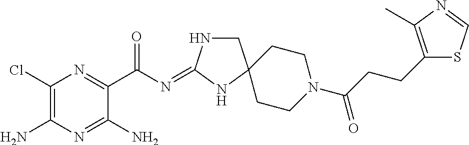 [SMILES: Cc1ncsc1CCC(=O)N1CCC2(CC1)CN/C(=N\C(=O)c1nc(Cl)c(N)nc1N)N2] | 478.01 |
| 184 | 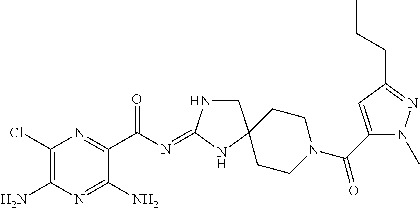 [SMILES: CCCc1cc(C(=O)N2CCC3(CC2)CN/C(=N\C(=O)c2nc(Cl)c(N)nc2N)N3)n(C)n1] | 475.08 |
| 185 | 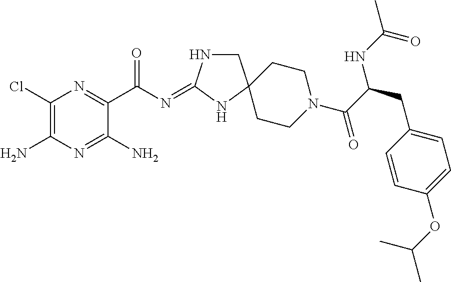 [SMILES: CC(=O)N[C@@H](Cc1ccc(OC(C)C)cc1)C(=O)N1CCC2(CC1)CN/C(=N\C(=O)c1nc(Cl)c(N)nc1N)N2] | 572.09 |
| 186 | 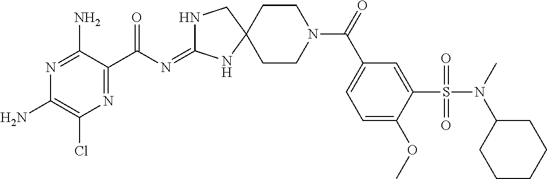 [SMILES: COc1ccc(C(=O)N2CCC3(CC2)CN/C(=N\C(=O)c2nc(Cl)c(N)nc2N)N3)cc1S(=O)(=O)N(C)C1CCCCC1] | 634.09 |
| 187 | 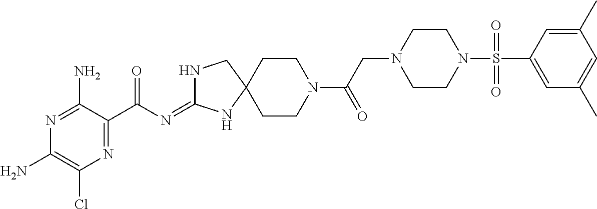 [SMILES: Cc1cc(C)cc(S(=O)(=O)N2CCN(CC(=O)N3CCC4(CC3)CN/C(=N\C(=O)c3nc(Cl)c(N)nc3N)N4)CC2)c1] | 619.12 |
| 188 | 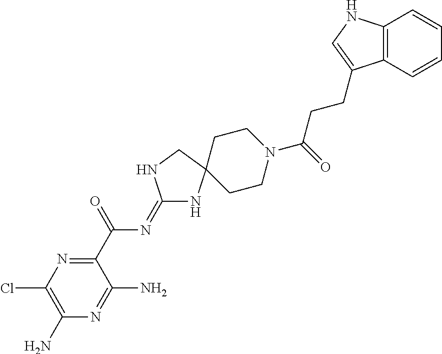 [SMILES: Nc1nc(N)c(C(=O)/N=C2\NCC3(CCN(C(=O)CCc4c[nH]c5ccccc45)CC3)N2)nc1Cl] | 496.02 |
| 189 | 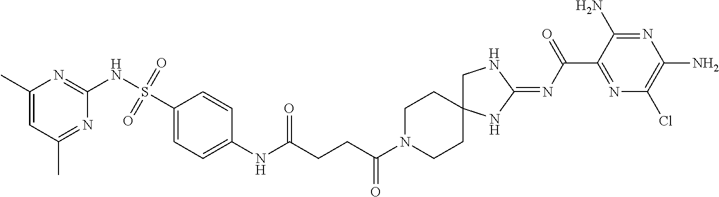 [SMILES: Cc1cc(C)nc(NS(=O)(=O)c2ccc(NC(=O)CCC(=O)N3CCC4(CC3)CN/C(=N\C(=O)c3nc(Cl)c(N)nc3N)N4)cc2)n1] | 685.08 |
| 190 | 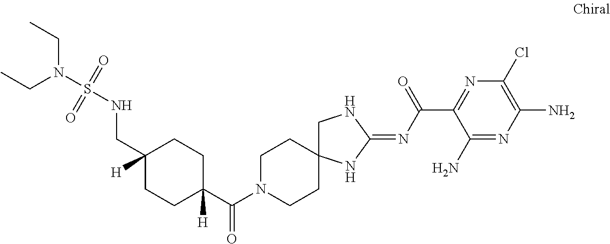 [SMILES: CCN(CC)S(=O)(=O)NC[C@H]1CC[C@@H](C(=O)N2CCC3(CC2)CN/C(=N\C(=O)c2nc(Cl)c(N)nc2N)N3)CC1] | 599.2 |
| 191 | 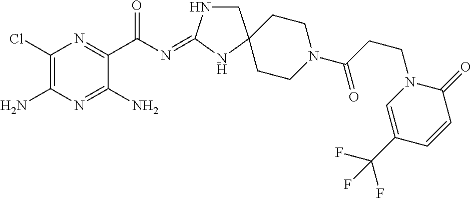 [SMILES: Nc1nc(N)c(C(=O)/N=C2\NCC3(CCN(C(=O)CCn4cc(C(F)(F)F)ccc4=O)CC3)N2)nc1Cl] | 542.01 |
| 192 | 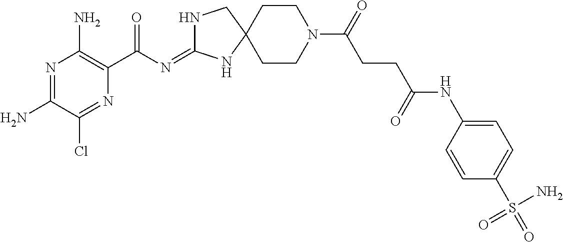 [SMILES: Nc1nc(N)c(C(=O)/N=C2\NCC3(CCN(C(=O)CCC(=O)Nc4ccc(S(N)(=O)=O)cc4)CC3)N2)nc1Cl] | 579.03 |
| 193 | 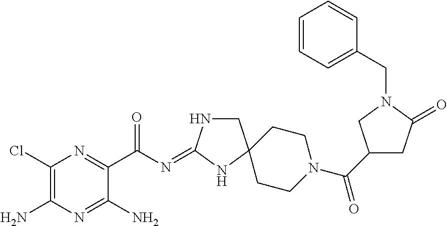 [SMILES: Nc1nc(N)c(C(=O)/N=C2\NCC3(CCN(C(=O)C4CC(=O)N(Cc5ccccc5)C4)CC3)N2)nc1Cl] | 526.05 |
| 194 | 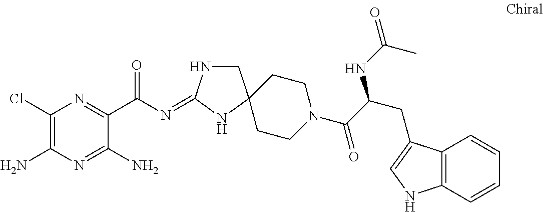 [SMILES: CC(=O)N[C@@H](Cc1c[nH]c2ccccc12)C(=O)N1CCC2(CC1)CN/C(=N\C(=O)c1nc(Cl)c(N)nc1N)N2] | 553.09 |
| 195 | 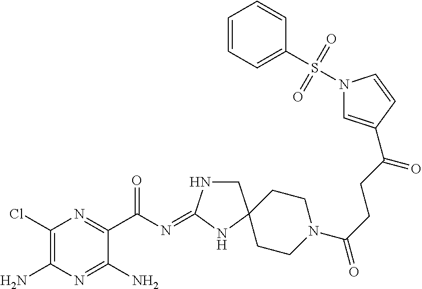 [SMILES: Nc1nc(N)c(C(=O)/N=C2\NCC3(CCN(C(=O)CCC(=O)c4ccn(S(=O)(=O)c5ccccc5)c4)CC3)N2)nc1Cl] | 614.3 |
| 196 | 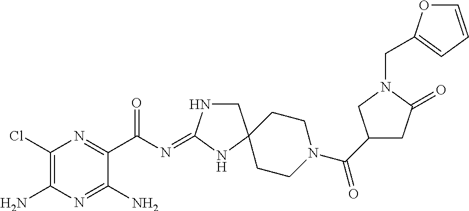 [SMILES: Nc1nc(N)c(C(=O)/N=C2\NCC3(CCN(C(=O)C4CC(=O)N(Cc5ccco5)C4)CC3)N2)nc1Cl] | 516.06 |
| 197 | 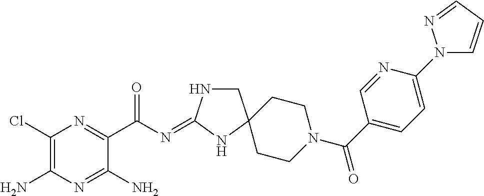 [SMILES: Nc1nc(N)c(C(=O)/N=C2\NCC3(CCN(C(=O)c4ccc(-n5cccn5)nc4)CC3)N2)nc1Cl] | 496.01 |
| 198 | 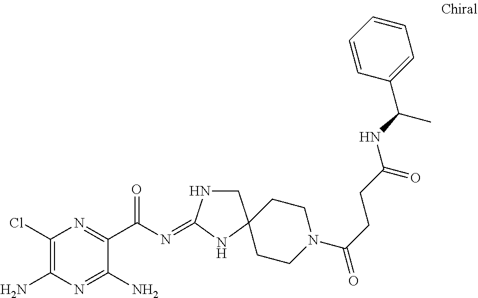 [SMILES: C[C@@H](NC(=O)CCC(=O)N1CCC2(CC1)CN/C(=N\C(=O)c1nc(Cl)c(N)nc1N)N2)c1ccccc1] | 528.04 |
| 199 | 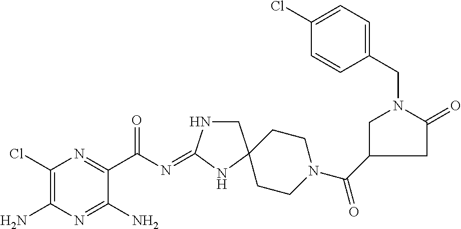 [SMILES: Nc1nc(N)c(C(=O)/N=C2\NCC3(CCN(C(=O)C4CC(=O)N(Cc5ccc(Cl)cc5)C4)CC3)N2)nc1Cl] | 560.14 |
| 200 | 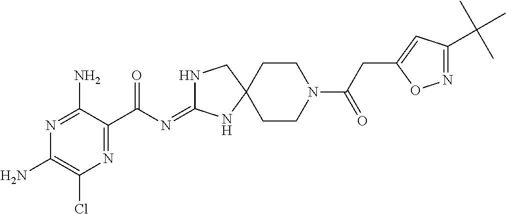 [SMILES: CC(C)(C)c1cc(CC(=O)N2CCC3(CC2)CN/C(=N\C(=O)c2nc(Cl)c(N)nc2N)N3)on1] | 490.05 |
| 201 | 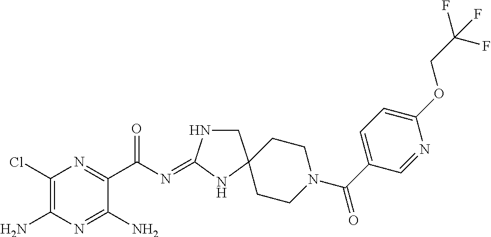 [SMILES: Nc1nc(N)c(C(=O)/N=C2\NCC3(CCN(C(=O)c4ccc(OCC(F)(F)F)nc4)CC3)N2)nc1Cl] | 528.06 |
| 202 | 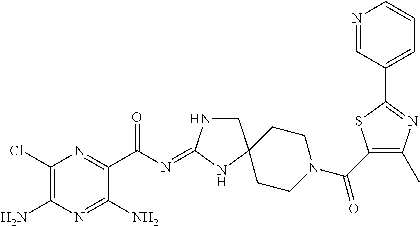 [SMILES: Cc1nc(-c2cccnc2)sc1C(=O)N1CCC2(CC1)CN/C(=N\C(=O)c1nc(Cl)c(N)nc1N)N2] | 527.02 |
| 203 | 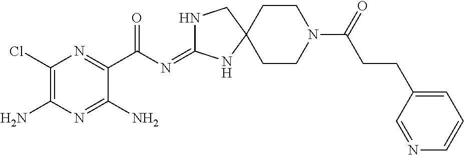 [SMILES: Nc1nc(N)c(C(=O)/N=C2\NCC3(CCN(C(=O)CCc4cccnc4)CC3)N2)nc1Cl] | 458.1 |
| 204 | 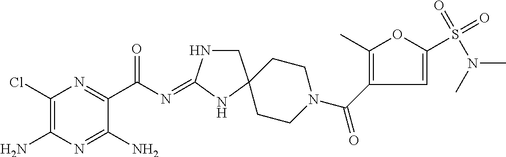 [SMILES: Cc1oc(S(=O)(=O)N(C)C)cc1C(=O)N1CCC2(CC1)CN/C(=N\C(=O)c1nc(Cl)c(N)nc1N)N2] | 540.02 |
| 205 | 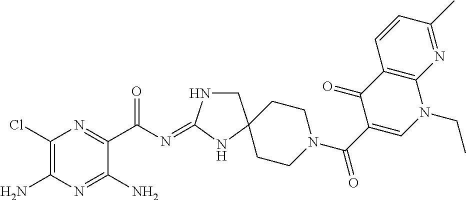 [SMILES: CCn1cc(C(=O)N2CCC3(CC2)CN/C(=N\C(=O)c2nc(Cl)c(N)nc2N)N3)c(=O)c2ccc(C)nc21] | 539.11 |
| 206 | 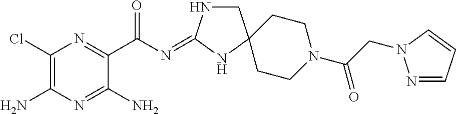 [SMILES: Nc1nc(N)c(C(=O)/N=C2\NCC3(CCN(C(=O)Cn4cccn4)CC3)N2)nc1Cl] | 433.05 |
| 207 | 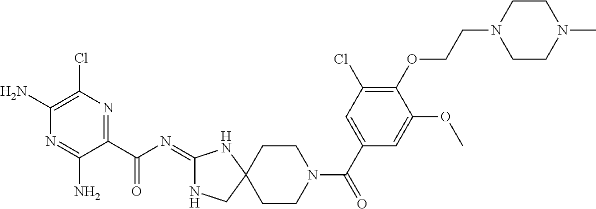 [SMILES: COc1cc(C(=O)N2CCC3(CC2)CN/C(=N\C(=O)c2nc(Cl)c(N)nc2N)N3)cc(Cl)c1OCCN1CCN(C)CC1] | 635.19 |
| 208 | 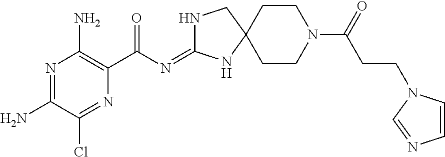 [SMILES: Nc1nc(N)c(C(=O)/N=C2\NCC3(CCN(C(=O)CCn4ccnc4)CC3)N2)nc1Cl] | 447.09 |
| 209 | 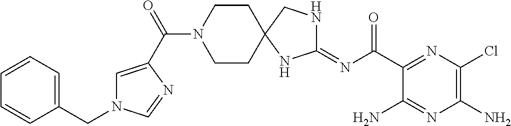 [SMILES: Nc1nc(N)c(C(=O)/N=C2\NCC3(CCN(C(=O)c4cn(Cc5ccccc5)cn4)CC3)N2)nc1Cl] | 509.09 |
| 210 | 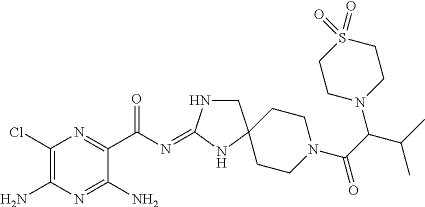 [SMILES: CC(C)C(C(=O)N1CCC2(CC1)CN/C(=N\C(=O)c1nc(Cl)c(N)nc1N)N2)N1CCS(=O)(=O)CC1] | 542.00 |
| 211 | 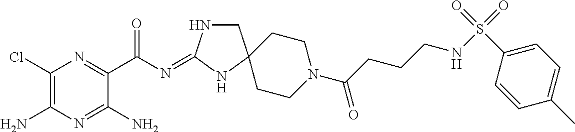 [SMILES: Cc1ccc(S(=O)(=O)NCCCC(=O)N2CCC3(CC2)CN/C(=N\C(=O)c2nc(Cl)c(N)nc2N)N3)cc1] | 564.06 |
| 212 | 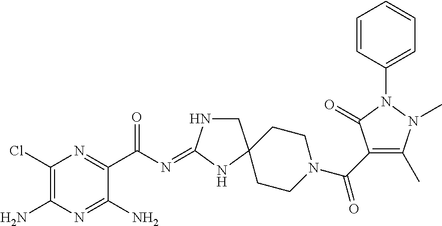 [SMILES: Cc1c(C(=O)N2CCC3(CC2)CN/C(=N\C(=O)c2nc(Cl)c(N)nc2N)N3)c(=O)n(-c2ccccc2)n1C] | 539.11 |
| 213 | 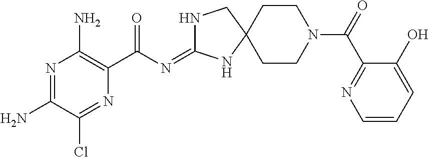 [SMILES: Nc1nc(N)c(C(=O)/N=C2\NCC3(CCN(C(=O)c4ncccc4O)CC3)N2)nc1Cl] | 445.96 |
| 214 | 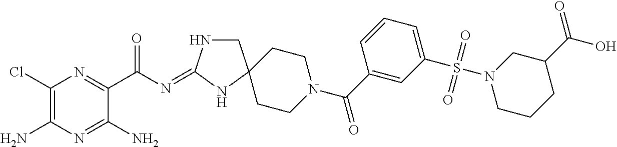 [SMILES: Nc1nc(N)c(C(=O)/N=C2\NCC3(CCN(C(=O)c4cccc(S(=O)(=O)N5CCCC(C(=O)O)C5)c4)CC3)N2)nc1Cl] | 620.1 |
| 215 | 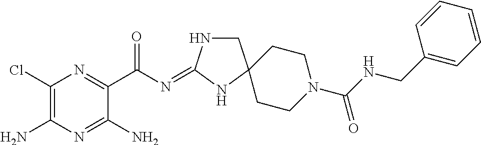 [SMILES: Nc1nc(N)c(C(=O)/N=C2\NCC3(CCN(C(=O)NCc4ccccc4)CC3)N2)nc1Cl] | 458.1 |
| 216 | 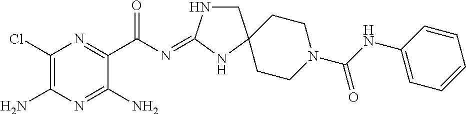 [SMILES: Nc1nc(N)c(C(=O)/N=C2\NCC3(CCN(C(=O)Nc4ccccc4)CC3)N2)nc1Cl] | |
| 217 | 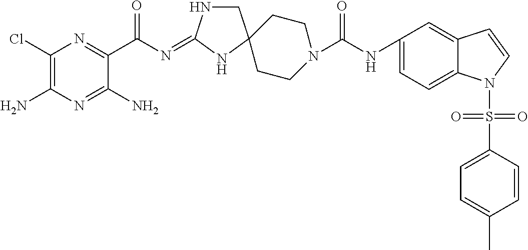 [SMILES: Cc1ccc(S(=O)(=O)n2ccc3cc(NC(=O)N4CCC5(CC4)CN/C(=N\C(=O)c4nc(Cl)c(N)nc4N)N5)ccc32)cc1] | 637.1 |
| 218 | 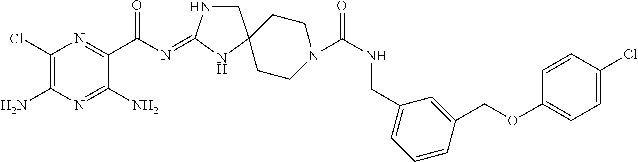 [SMILES: Nc1nc(N)c(C(=O)/N=C2\NCC3(CCN(C(=O)NCc4cccc(COc5ccc(Cl)cc5)c4)CC3)N2)nc1Cl] | 598.05 |
| 219 | 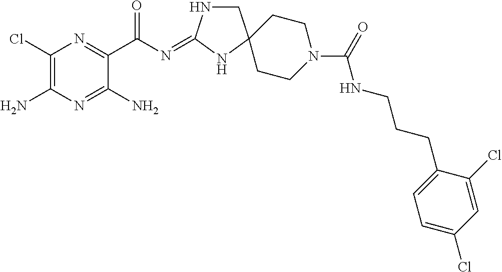 [SMILES: Nc1nc(N)c(C(=O)/N=C2\NCC3(CCN(C(=O)NCCCc4ccc(Cl)cc4Cl)CC3)N2)nc1Cl] | 554.0 |
| 220 | 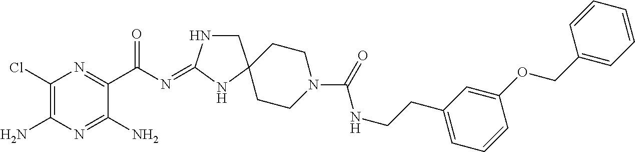 [SMILES: Nc1nc(N)c(C(=O)/N=C2\NCC3(CCN(C(=O)NCCc4cccc(OCc5ccccc5)c4)CC3)N2)nc1Cl] | 578.2 |
| 221 | 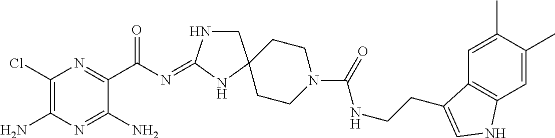 [SMILES: Cc1cc2[nH]cc(CCNC(=O)N3CCC4(CC3)CN/C(=N\C(=O)c3nc(Cl)c(N)nc3N)N4)c2cc1C] | 539.2 |
| 222 | 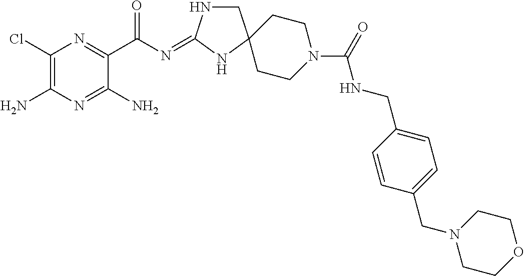 [SMILES: Nc1nc(N)c(C(=O)/N=C2\NCC3(CCN(C(=O)NCc4ccc(CN5CCOCC5)cc4)CC3)N2)nc1Cl] | 557.2 |
| 223 | 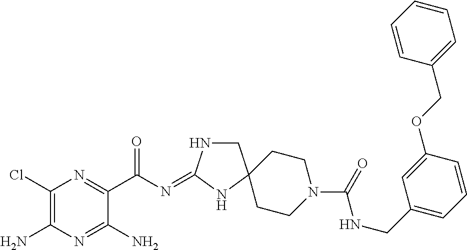 [SMILES: Nc1nc(N)c(C(=O)/N=C2\NCC3(CCN(C(=O)NCc4cccc(OCc5ccccc5)c4)CC3)N2)nc1Cl] | 564.1 |
| 224 | 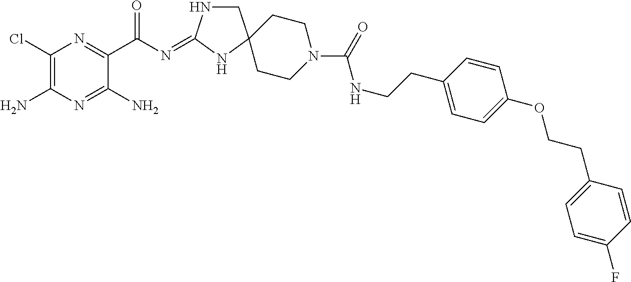 [SMILES: Nc1nc(N)c(C(=O)/N=C2\NCC3(CCN(C(=O)NCCc4ccc(OCCc5ccc(F)cc5)cc4)CC3)N2)nc1Cl] | 610.2 |
| 225 | 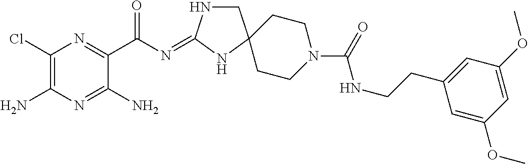 [SMILES: COc1cc(CCNC(=O)N2CCC3(CC2)CN/C(=N\C(=O)c2nc(Cl)c(N)nc2N)N3)cc(OC)c1] | 532.1 |
| 226 | 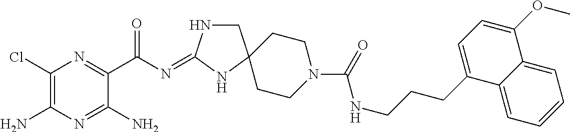 [SMILES: COc1ccc(CCCNC(=O)N2CCC3(CC2)CN/C(=N\C(=O)c2nc(Cl)c(N)nc2N)N3)c2ccccc12] | 566.1 |
| 227 | 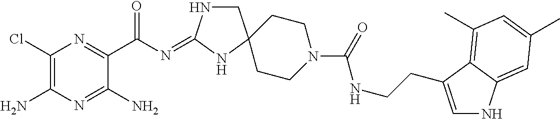 [SMILES: Cc1cc(C)c2c(CCNC(=O)N3CCC4(CC3)CN/C(=N\C(=O)c3nc(Cl)c(N)nc3N)N4)c[nH]c2c1] | 539.2 |
| 228 | 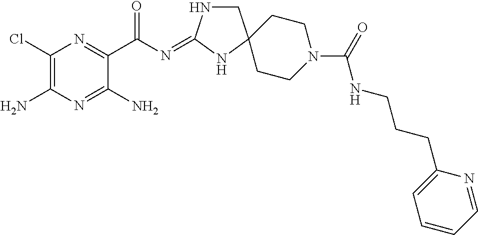 [SMILES: Nc1nc(N)c(C(=O)/N=C2\NCC3(CCN(C(=O)NCCCc4ccccn4)CC3)N2)nc1Cl] | 487.1 |
| 229 | 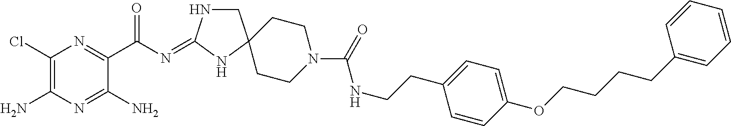 [SMILES: Nc1nc(N)c(C(=O)/N=C2\NCC3(CCN(C(=O)NCCc4ccc(OCCCCc5ccccc5)cc4)CC3)N2)nc1Cl] | 620.2 |
| 230 | 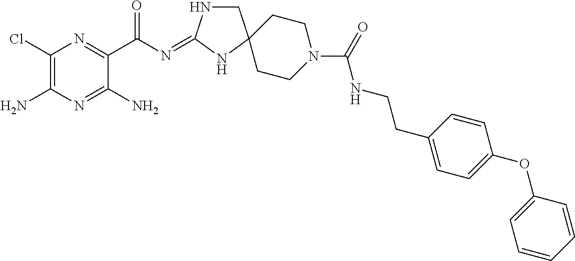 [SMILES: Nc1nc(N)c(C(=O)/N=C2\NCC3(CCN(C(=O)NCCc4ccc(Oc5ccccc5)cc4)CC3)N2)nc1Cl] | 564.2 |
| 231 | 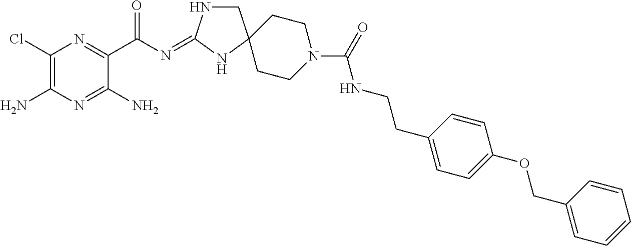 [SMILES: Nc1nc(N)c(C(=O)/N=C2\NCC3(CCN(C(=O)NCCc4ccc(OCc5ccccc5)cc4)CC3)N2)nc1Cl] | 578.2 |
| 232 | 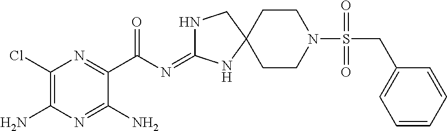 [SMILES: Nc1nc(N)c(C(=O)/N=C2\NCC3(CCN(S(=O)(=O)Cc4ccccc4)CC3)N2)nc1Cl] | 478.98 |
| 233 | 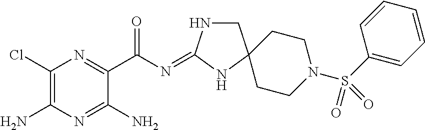 [SMILES: Nc1nc(N)c(C(=O)/N=C2\NCC3(CCN(S(=O)(=O)c4ccccc4)CC3)N2)nc1Cl] | |
| 234 | 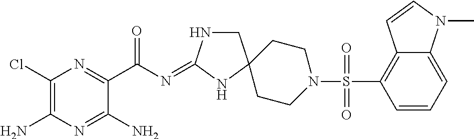 [SMILES: Cn1ccc2c(S(=O)(=O)N3CCC4(CC3)CN/C(=N\C(=O)c3nc(Cl)c(N)nc3N)N4)cccc21] | 517.9 |
| 235 | 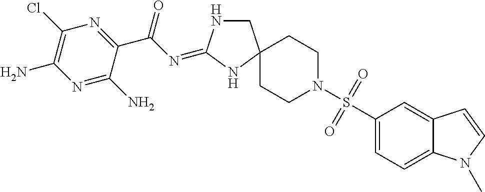 [SMILES: Cn1ccc2cc(S(=O)(=O)N3CCC4(CC3)CN/C(=N\C(=O)c3nc(Cl)c(N)nc3N)N4)ccc21] | 518.1 |
| 236 | 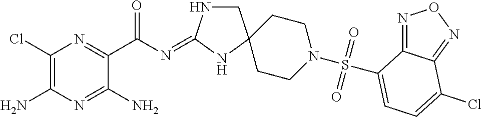 [SMILES: Nc1nc(N)c(C(=O)/N=C2\NCC3(CCN(S(=O)(=O)c4ccc(Cl)c5nonc45)CC3)N2)nc1Cl] | 540.9 |
| 237 | 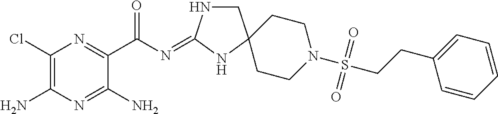 [SMILES: Nc1nc(N)c(C(=O)/N=C2\NCC3(CCN(S(=O)(=O)CCc4ccccc4)CC3)N2)nc1Cl] | 493.1 |
| 238 | 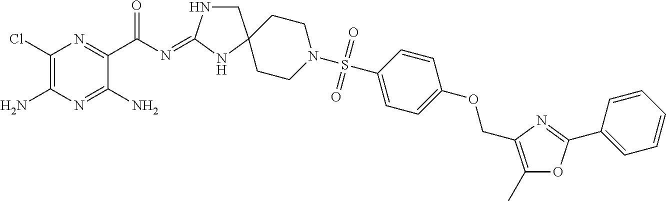 [SMILES: Cc1oc(-c2ccccc2)nc1COc1ccc(S(=O)(=O)N2CCC3(CC2)CN/C(=N\C(=O)c2nc(Cl)c(N)nc2N)N3)cc1] | 652.2 |
| 239 | 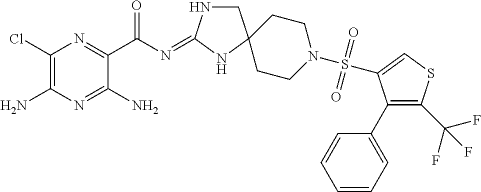 [SMILES: Nc1nc(N)c(C(=O)/N=C2\NCC3(CCN(S(=O)(=O)c4csc(C(F)(F)F)c4-c4ccccc4)CC3)N2)nc1Cl] | 615.1 |
| 240 | 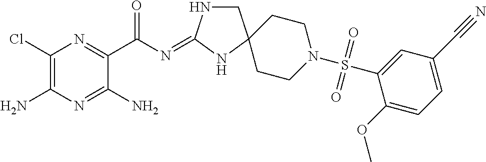 [SMILES: COc1ccc(C#N)cc1S(=O)(=O)N1CCC2(CC1)CN/C(=N\C(=O)c1nc(Cl)c(N)nc1N)N2] | 520.1 |
| 241 | 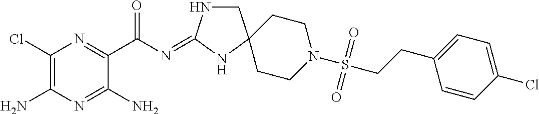 [SMILES: Nc1nc(N)c(C(=O)/N=C2\NCC3(CCN(S(=O)(=O)CCc4ccc(Cl)cc4)CC3)N2)nc1Cl] | 527.0 |
| 242 | 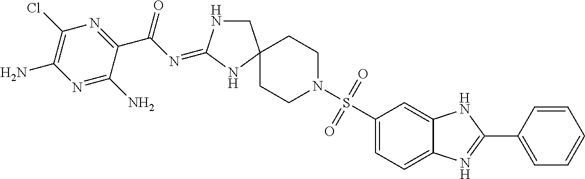 [SMILES: Nc1nc(N)c(C(=O)/N=C2\NCC3(CCN(S(=O)(=O)c4ccc5nc(-c6ccccc6)[nH]c5c4)CC3)N2)nc1Cl] | 581.1 |
| 243 | 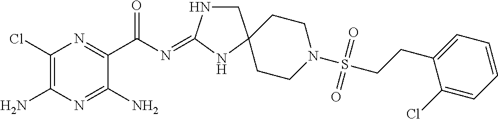 [SMILES: Nc1nc(N)c(C(=O)/N=C2\NCC3(CCN(S(=O)(=O)CCc4ccccc4Cl)CC3)N2)nc1Cl] | 527.1 |
| 244 | 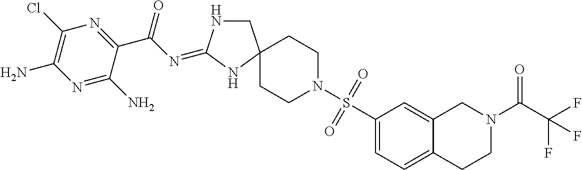 [SMILES: Nc1nc(N)c(C(=O)/N=C2\NCC3(CCN(S(=O)(=O)c4ccc5c(c4)CN(C(=O)C(F)(F)F)CC5)CC3)N2)nc1Cl] | 616.1 |
| 245 | 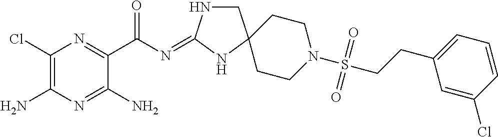 [SMILES: Nc1nc(N)c(C(=O)/N=C2\NCC3(CCN(S(=O)(=O)CCc4cccc(Cl)c4)CC3)N2)nc1Cl] | 527.0 |
| 246 | 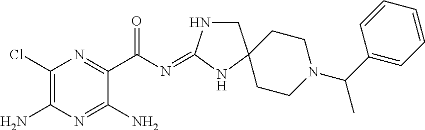 [SMILES: CC(c1ccccc1)N1CCC2(CC1)CN/C(=N\C(=O)c1nc(Cl)c(N)nc1N)N2] | 429 |
| 247 | 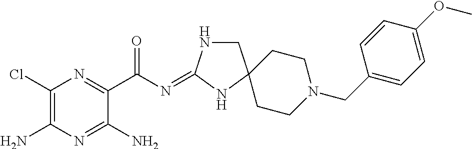 [SMILES: COc1ccc(CN2CCC3(CC2)CN/C(=N\C(=O)c2nc(Cl)c(N)nc2N)N3)cc1] | 445 |
| 248 | 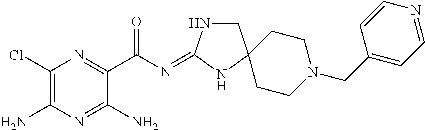 [SMILES: Nc1nc(N)c(C(=O)/N=C2\NCC3(CCN(Cc4ccncc4)CC3)N2)nc1Cl] | 416 |
| 249 | 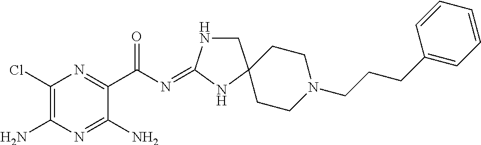 [SMILES: Nc1nc(N)c(C(=O)/N=C2\NCC3(CCN(CCCc4ccccc4)CC3)N2)nc1Cl] | 443 |
| 250 | 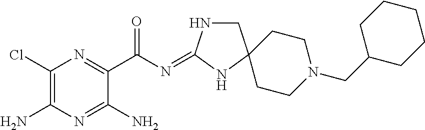 [SMILES: Nc1nc(N)c(C(=O)/N=C2\NCC3(CCN(CC4CCCCC4)CC3)N2)nc1Cl] | 421 |
| 251 | 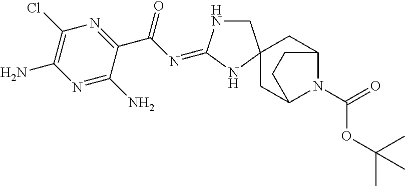 [SMILES: CC(C)(C)OC(=O)N1C2CCC1CC1(CN/C(=N\C(=O)c3nc(Cl)c(N)nc3N)N1)C2] | 451 |
| 252 | 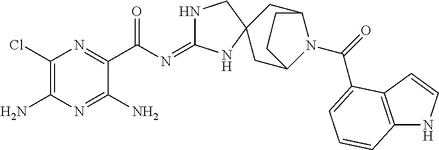 [SMILES: Nc1nc(N)c(C(=O)/N=C2\NCC3(CC4CCC(C3)N4C(=O)c3cccc4[nH]ccc34)N2)nc1Cl] | 494.15 |
| 253 | 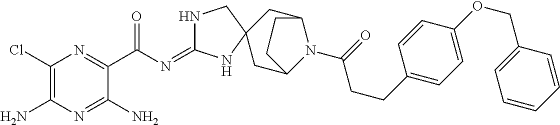 [SMILES: Nc1nc(N)c(C(=O)/N=C2\NCC3(CC4CCC(C3)N4C(=O)CCc3ccc(OCc4ccccc4)cc3)N2)nc1Cl] | 589.20 |

### General Conditions

LCMS are recorded using a Phenomenex Gemini 50 mm×3.0 mm, 3 um column. Low pH methods use a gradient of 5-95% acetonitrile in water −0.1% TFA, high pH methods use 5-95% acetonitrile in water −0.1% NH₃. [M+H]⁺ refer to monoisotopic molecular weights.

9-BBN 9-Borabicyclo[3.3.1]nonane
DBU Diazabicyclo[5.4.0]undec-7-ene
DMF dimethylformamide
DMSO dimethyl sulfoxide
DCM dichloromethane
DEAD diethyl azodicarboxylate
DIAD diisopropyl azodicarboxylate
DIPEA diisopropylethylamine
EDCI 1-ethyl-3-(3′-dimethylaminopropyl)carbodiimide
Er0Ac ethyl acetate
HATU 2-(7-Aza-1H-benzotriazole-1-yl)-1,1,3,3-tetramethyluronium hexafluorophosphate
HPLC high performance liquid chromatography
IPA Isopropyl alcohol (iso-propanol)
MeOH methanol
MEMCl 2-methoxyethoxymethyl chloride
NMR nuclear magnetic resonance
PS polymer supported
PPTS Pyridinium para-toluenesulfonate
PEAX PE-anion exchange (e.g. Isolute® PE-AX columns from Biotage)
SCX-2 strong cation exchange (e.g. Isolute® SCX-2 columns from Biotage)
TEA triethylamine
THF tetrahydrofuran
TFA trifluoroacetic acid

### Preparation of Examples

For clarity in describing the Examples described below. Examples 2, 9, and 10 are racemic mixtures. Examples 4, 13 and 29 are mixtures of diastereomers. Examples 24 and 25 are single enantiomers wherein the stereochemistry of the unassigned stereocentre is not determined. All other examples are single enantiomers of defined stereochemistry.

Where not stated, the compounds are recovered from reaction mixtures and purified using conventional techniques such as flash chromatography, filtration, recrystallisation and trituration.

### Example 1

### 3,5-Diamino-6-chloro-pyrazine-2-carboxylic acid [4,4-dimethyl-imidazolidin-(2Z)-ylidene]-amide

A suspension of 1-(3,5-diamino-6-chloro-pyrazine-2-carbonyl)-2-methyl-isothiourea (Intermediate A) (0.2 g, 0.517 mmol) in EtOH (2 ml) is treated with triethylamine (0.029 ml, 0.258 mmol) followed by 1,2-diamino-2-methylpropane (0.07 ml, 0.672 mmol) and stirred at reflux overnight. The resulting suspension is filtered under vacuum to afford the title compound as a pale yellow solid; [M+H]⁺284

### Example 2

### 3,5-Diamino-6-chloro-pyrazine-2-carboxylic acid [4-methyl-imidazolidin-(2Z)-ylidene]-amide

This compound is prepared analogously to Example 1 by replacing 1,2-diamino-2-methylpropane with 1,2,diaminopropane; [M+H]⁺270.

### Example 3

### 3,5-Diamino-6-chloro-pyrazine-2-carboxylic acid [1-methyl-imidazolidin-(2Z)-ylidene]-amide

This compound is prepared analogously to Example 1 by replacing 1,2-diamino-2-methylpropane with N-methylenediamine; [M+H]⁺270.

### Example 4

### 3,5-Diamino-6-chloro-pyrazine-2-carboxylic acid (4,5-diphenyl-imidazolidin-2-ylidene)-amide

This compound is prepared analogously to Example 1 by replacing 1,2-diamino-2-methylpropane with 1,2 diphenylethylene diamine; [M+H]⁺408.

### Example 5

### (4-{2-[(Z)-3,5-Diamino-6-chloro-pyrazine-2-carbonylimino]-imidazolidin-4-yl}-butyl)-carbamic acid benzyl ester

This compound is prepared analogously to Example 1 by replacing 1,2-diamino-2-methylpropane with ((S)-5,6-Diamino-hexyl)-carbamic acid benzyl ester (Intermediate B); [M+H]⁺461.

### Example 6

### 3,5-Diamino-6-chloro-pyrazine-2-carboxylic acid [1-[4-(4-methoxy-phenyl)-butyl]-imidazolidin-(2Z)-ylidene]-amide

This compound is prepared analogously to Example 1 by replacing 1,2-diamino-2-methylpropane with N*1*-[4-(4-methoxy-phenyl)-butyl]-ethane-1,2-diamine (Intermediate C); [M+H]⁺418.

### Example 7

### 3,5-Diamino-6-chloro-pyrazine-2-carboxylic acid [1-[4-(4-hydroxy-phenyl)-butyl]-imidazolidin-(2Z)-ylidene]-amide

This compound is prepared analogously to Example 1 by replacing 1,2-diamino-2-methylpropane with 4-[4-(2-amino-ethylamino)-butyl]-phenol (Intermediate C); [M+H]⁺404.

### Example 8

### 3,5-Diamino-6-chloro-pyrazine-2-carboxylic acid [(S)-4-(4-methoxy-benzyl)-imidazolidin-(2Z)-ylidene]-amide

This compound is prepared analogously to Example 1 by replacing 1,2-diamino-2-methylpropane with (S)-3-(4-methoxy-phenyl)-propane-1,2 diamine (Intermediate D); [M+H]⁺376.

### Example 9

### 3,5-Diamino-6-chloro-pyrazine-2-carboxylic acid [4-(3,4-dichloro-phenyl)-imidazolidin-(2Z)-ylidene]-amide

This compound is prepared analogously to Example 1 by replacing 1,2-diamino-2-methylpropane with 1-(3,4-Dichloro-phenyl)-ethane-1,2-diamine (Intermediate E); [M+H]⁺400.

### Example 10

### 3-{2-[(Z)-3,5-Diamino-6-chloro-pyrazine-2-carbonylimino]-imidazolidin-4-yl}-propionic acid

This compound is prepared analogously to Example 1 by replacing 1,2-diamino-2-methylpropane with 4,5-Diaminopentanoic acid dihydrochloride (Intermediate F); [M+H]⁺328.

### Examples 2-10

These compounds are recovered from reaction mixtures and purified using conventional techniques such as flash chromatography, filtration, capture release resin or preparative HPLC.

### Example 11

### 3,5-Diamino-6-chloro-pyrazine-2-carboxylic acid (octahydro-benzoimidazol-2-ylidene)-amide

This compound is prepared analogously to Example 1 by replacing 1,2-diamino-2-methylpropane with cyclohexane-1,2-diamine. The reaction is carried out in propan-2-ol; [M+H]⁺310.

### Example 12

### 3,5-Diamino-6-chloro-pyrazine-2-carboxylic acid [8-benzyl-1,3,8-triazaspiro[4.5]dec-(2Z)-ylidene]-amide

4-Amino-1-benzyl-piperidine-4-carbonitrile (Intermediate G) (200 mg, 0.91 mmol) in dry propan-2-ol (10 ml) is treated with triethylamine (0.25 ml) followed by 1-(3,5-diamino-6-chloro-pyrazine-2-carbonyl)-2-methyl-isothiourea (Intermediate A) (355 mg, 0.91 mmol). The mixture is heated at 70° C. for 5 hours and then allowed to cool to room temperature. The precipitate is collected and washed with methanol to afford the title compound as a light yellow solid, 190 mg; [M+H]⁺415.

### Example 13

### 3,5-Diamino-6-chloro-pyrazine-2-carboxylic acid [4-[3-(4-methoxy-phenyl)-propyl]-imidazolidin-(2Z)-ylidene]-amide

This compound is prepared analogously to Example 12 by replacing 4-Amino-1-benzyl-piperidine-4-carbonitrile (Intermediate G) with 5-(4-methoxy-phenyl)-pentane-1,2-diamine (Intermediate I); [M+H]⁺404.

### Example 14

### 3,5-Diamino-6-chloro-pyrazine-2-carboxylic acid (tetrahydro-pyrimidin-2-ylidene)-amide

1-(3,5-Diamino-6-chloro-pyrazine-2-carbonyl)-2-methyl-isothiourea (Intermediate A) (1.0 g, 2.58 mmol) is suspended in propan-2-ol (10 ml) and 1,3-diaminopropane (0.32 ml, 3.9 mmol) is added. The mixture is heated at 60° C. for 18 hours and then allowed to cool to room temperature and the solids present are collected by filtration. The solids are washed with THF and MeOH to yield the title compound as a yellow solid; [M+H]⁺270.

### Example 15

### 3,5-diamino-6-chloro-N-(1H-pyrrolo[1,2-c]imidazol-3 (2H,5H,6H,7H,7aH)-ylidene)pyrazine-2-carboxamide

1-(3,5-Diamino-6-chloro-pyrazine-2-carbonyl)-2-methyl-isothiourea (Intermediate A) (195 mg, 0.5 mmol) is suspended in propan-2-ol (10 ml) and (S)-2-(aminomethyl)pyrrolidine (100 mg, 1 mmol) is added. The mixture is heated at 60° C. for 18 hours, allowed to cool to room temperature and the precipitate is removed by filtration. The filtrate is concentrated in vacuo and the residue purified by chromatography (SiO₂, DCM/MeOH) to afford the title compound as a light, yellow gum; [M+H]⁺296.

### Example 16

### 3,5-Diamino-6-chloro-pyrazine-2-carboxylic acid [1,3-d]aza-spiro[4.4]non-(2Z)-ylidene]-amide

A solution of crude 1-aminomethyl-cyclopentylamine (Intermediate J) (80 mg, 0.70 mmol) in propan-2-ol (1.0 ml) is added to a suspension of 1-(3,5-diamino-6-chloro-pyrazine-2-carbonyl)-2-methyl-isothiourea (Intermediate A) (208 mg, 0.54 mmol) in propan-2-ol (1.08 ml) and heated at 70° C. for 2 days. After cooling to room temperature, the reaction mixture is filtered under vacuum, and the solid is rinsed with MeOH. The filtrate is concentrated in vacuo to afford a bright yellow residue which is loaded onto a SCX-2 cartridge and eluted with 33% NH₃ (4 drops) in MeOH (5 ml×2). The methanolic ammonia fractions are combined and concentrated in vacuo. Purification using mass directed preparative LCMS eluting with 95% Water+0.1% NH₃: 5% Acetonitrile to affords the title compound; [M+H]⁺310.

### Example 17

### 3,5-Diamino-6-chloro-pyrazine-2-carboxylic acid [(R)-4-[3-(4-hydroxy-phenyl)-propyl]-imidazolidin-(2E)-ylidene]-amide

To a stirred solution of (4-((R)-4,5-Diamino-pentyl)-phenol (intermediate K) (1.5 g, 7.72 mmol) in propan-2-ol (100 ml) at 30° C. is added in one portion 1-(3,5-Diamino-6-chloro-pyrazine-2-carbonyl)-2-methyl-isothiourea (Intermediate A) and the reaction is heated at 30° C. for 18 hours followed by 50° C. for a further 18 hours. The reaction mixture is filtered hot and the filtrate solvent is removed in vacuo to afford a yellow foam. The foam is purified by chromatography (SiO₂, DCM/MeOH/5% NH₃) to afford the title compound; [M+H]⁺390.

### Example 18

### 3,5-Diamino-6-chloro-pyrazine-2-carboxylic acid [(S)-4-[3-(4-hydroxy-phenyl)-propyl]-imidazolidin-(2E)-ylidene]-amide

This compound is prepared analogously to Example 17 replacing (4-((R)-4,5-Diamino-pentyl)-phenol (Intermediate K) with 4-((S)-4,5-Diamino-pentyl)-phenol (intermediate L; [M+H]⁺390.

### Example 19

### 3,5-Diamino-6-chloro-pyrazine-2-carboxylic acid [(R)-4-{3-[4-((S)-2,3-dihydroxy-propoxy)-phenyl]-propyl}imidazolidin-(2Z)-ylidene]-amide

To a stirred solution of 3,5-Diamino-6-chloro-pyrazine-2-carboxylic acid [(R)-4-[3-(4-hydroxy-phenyl)-propyl]-imidazolidin-(2E)-ylidene]-amide (Ex. 17) (1.0 g, 2.57 mmol) in 1,4 dioxane (38 ml) at 50° C. is added in one portion 0.5 M KOH (5.3 ml, 2.7 mmol) followed by (S)-(−)-Glycidiol (0.170 ml, 2.57 mmol). The resulting mixture is heated at 50° C. for 18 hours and then further (S)-(−)-Glycidiol (0.07 ml, 1.05 mmol) is added in one portion. The resulting mixture is heated at 50° C. for 60 hours and then allowed to cool to room temperature. The solvent is removed in vacuo to afford an orange oil which is dissolved in EtOAc/MeOH 9:1 (100 ml) and washed with 1 M NaOH (50 ml). The organic layer is dried over Na₂SO₄ and the solvent is removed in vacuo to afford a brown/orange foam. Purification by chromatography (SiO₂, DCM/MeOH/NH₃) affords the title compound as a yellow foam; [M+H]⁺464; ¹H NMR (400 MHz, DMSO-d6): 1.65-1.40 (m, 4H), 2.52 (m, 2H), 3.13 (dd, J=9.6, 7.1 Hz, 1H), 3.42 (br d, J=4.7 Hz, 2H), 3.62 (dd, J=9.6, 9.6 Hz, 1H), 3.76 (m, 1H), 3.78 (m, 1H), 3.80 (m, 1H), 3.94 (dd, J=9.5, 4.0 Hz, 1H), 4.62 (br s, 1H), 4.89 (br s, 1H), 6.68 (br s, 2H), 6.82 (d, J=8.5 Hz, 2H), 7.09 (d, J=8.5 Hz, 2H), 7.2-6.0 (br s, 1H), 8.18 (br s, 1H), 9.3-7.5 (br s, 1H).

### Example 20

### 3,5-Diamino-6-chloro-pyrazine-2-carboxylic acid [(S)-4-{3-[4-((S)-2,3-dihydroxy-propoxy)-phenyl]-propyl}-imidazolidin-(2Z)-ylidene]-amide

To a solution of 3,5-Diamino-6-chloro-pyrazine-2-carboxylic acid [(S)-4-[3-(4-hydroxy-phenyl)-propyl]-imidazolidin-(2E)-ylidene]-amide (Example 18) (37.5 mg, 0.09 mmol) in Ethanol (2 ml) is added triethylamine (63 μl, 0.45 mmol) and (S)-glycidol (6.07 μl, 0.09 mmol). The resulting mixture is heated at reflux for 18 hours and then allowed to cool to room temperature. The reaction mixture is diluted with MeOH (1 ml) and purified on a Waters 3000 prep HPLC system, (Microsorb C18, Water (0.1% TFA): MeCN) to afford the title compound; [M+H]⁺464.

### Example 21

### (3,5-Diamino-6-chloro-pyrazine-2-carboxylic acid [(R)-4-{3-[4-((R)-2,3-dihydroxy-propoxy)-phenyl]propyl}-imidazolidin-(2Z)-ylidene]-amide

To a stirred solution of (R)-3-[4-((R)-4,5-Diamino-pentyl)-phenoxy]-propane-1,2-diol (Intermediate 0) (32.8 mg, 0.122 mmol) in propan-2-ol (3 ml) is added 1-(3,5-Diamino-6-chloro-pyrazine-2-carbonyl)-2-methyl-isothiourea (Intermediate A) (45.8 mg, 0.122 mmol) and the resultant reaction mixture is heated at 90° C. for 18 hours. The reaction is allowed to cool to room temperature and diluted with DMSO (1.5 ml) and purified on a Waters 3000 preperative HPLC system (Microsorb™ C18, Water (0.1% TFA): MeCN). The fractions containing product are passed through a 1 g SCX-2 cartridge which is eluted with 1:1 Water:MeCN (20 ml), MeCN (20 ml) and 7M NH₃ in MeOH (20 ml). The ammonia elutions are concentrated in vacuo to afford the title compound; [M+H]⁺464.

### Example 22

### 3,5-Diamino-6-chloro-pyrazine-2-carboxylic acid [(S)-4-{3-[4-((R)-2,3-dihydroxy-propoxy)-phenyl]propyl}-imidazolidin-(2Z)-ylidene]-amide trifluoroacetate

This compound is prepared analogously to Example 21 replacing (R)-3-[4-((R)-4,5-Diamino-pentyl)-phenoxy]-propane-1,2-diol (Intermediate O) with (R)-3-[4-((S)-4,5-Diamino-pentyl)-phenoxy]-propane-1,2-diol (Intermediate P); [M+H]⁺464.

### Example 23

### 3,5-Diamino-6-chloro-pyrazine-2-carboxylic acid [(R)-4-{3-[4-(2-morpholin-4-yl-2-oxo-ethoxy)-phenyl]-propyl}-imidazolidin-(2Z)-ylidene]-amide

This compound is prepared analogously to Example 21 replacing (R)-3-[4-((R)-4,5-Diamino-pentyl)-phenoxy]-propane-1,2-diol (Intermediate 0) with 2-[4-((R)-4,5-Diamino-pentyl)-phenoxy]-1-morpholin-4-yl-ethanone (Intermediate Q); [M+H]⁺517.

### Examples 24 and 25

Both Enantiomers of 3,5-Diamino-6-chloro-pyrazine-2-carboxylic acid [4-[3-(4-methoxy-phenyl)-butyl]-imidazolidin-(2Z)-ylidene]-amide

The racemate of these compounds is prepared analogously to Example 12 replacing 4-Amino-1-benzyl-piperidine-4-carbonitrile (Intermediate G) with 5-(4-methoxy-phenyl)-hexane-1,2-diamine (Intermediate K). The enantiomers are separated by chiral HPLC:

Mobile phase: 100% EtOH (0.2% IPAm)

Column: Chirapak-AD 25 cm×4.6 mm i.d

Flow rate: 1 ml/min

UV 280 nM

Concentration 1 mg/mL

Inj Vol 10 μL

### Example 26

### 3,5-Diamino-6-chloro-pyrazine-2-carboyxlic acid [(S)-4-(4-benzyloxy-2,2-dimethyl-butyl-imidazolidin-(2Z)-ylidene]-amide

### Step 1

DEAD (4.49 ml, 28 mmol) is added to a stirred suspension of ((S)-5-benzyloxy-1-hydroxymethyl-3,-3-dimethyl-pentyl)-carbamic acid tert-butyl ester (prepared as described in EP 0702004 A2, Rueger et al., 10 g, 0.028 mmol), phthalimide (4.19 g, 0.028 mmol) and PS-triphenylphosphine (29.8 g, 56 mmol) in THF (500 ml), and the resulting reaction is stirred at room temperature for 3 days. The reaction is filtered to remove the PS-triphenylphosphine resin and the resin is washed with EtOAc (2×50 ml). The solvent is removed in vacuo and the residue is purified by flash chromatography (SiO₂, EtOAc/iso-hexane) to afford [(S)-5-benzyloxy-1-(1,3-dioxo-1,3-dihydro-isoindol-2-ylmethyl)-3,3-dimethyl-pentyl]-carbamic acid tert-butyl ester as a white solid; [M+H]⁺481.

### Step 2

Hydrazine (66.6 ml of a 1M solution in THF, 66.6 mmol) is added to a suspension of [(S)-5-benzyloxy-1-(1,3-dioxo-1,3-dihydro-isoindol-2-ylmethyl)-3,3-dimethyl-pentyl]-carbamic acid tert-butyl ester (4 g, 8.32 mmol) in ethanol (100 ml), and the resulting solution is heated at 40° C. overnight. A fluffy white precipitate forms. The reaction is allowed to cool to room temperature and diethyl ether (100 ml) is added and the resulting white suspension cooled at 0° C. for 30 minutes. The white precipitate is removed by filtration and the solvent removed in vacuo. The residue is then stirred with diethyl ether (100 ml) for 1 hour, filtered and the solvent is removed in vacuo to afford ((S)-1-Aminomethyl-5-benzyloxy-3,3-dimethyl-pentyl)-carbamic acid tert-butyl ester as a pale yellow oil; [M+H]⁺351.

### Step 3

Iodotrimethylsilane (1.63 ml, 11.94 mmol) is added dropwise to a solution of ((S)-1-Aminomethyl-5-benzyloxy-3,3-dimethyl-pentyl)-carbamic acid tert-butyl ester (2.79 g, 7.96 mmol) in DCM (30 ml) and the resulting yellow solution is stirred for 1 hour at room temperature. The reaction is filtered and the filtrate diluted with DCM (50 ml) and washed with 2 M NaOH (100 ml). The aqueous layer is allowed to stand overnight and any product which has oiled out of solution is extracted into EtOAc (100 ml). The organic layers are combined, dried over MgSO₄, and the solvent is removed in vacuo to yield (S)-Benzyloxy-4,4-dimethyl-hexane-1,2-diamine as a pale yellow oil; [M+H]⁺251.

### Step 4

A suspension of 1-(3,5-diamino-6-chloro-pyrazine-2-carbonyl)-2-methyl-isothiourea (Intermediate A) (2.56 g, 6.87 mmol) and (S)-Benzyloxy-4,4-dimethyl-hexane-1,2-diamine (1.72 g, 6.87 mmol) in propan-2-ol (50 ml) is heated at 90° C. for 3 hours. The reaction is allowed to cool to room temperature, filtered to remove any insoluble material and the filter paper is washed with MeOH (50 ml). The filtrate is loaded on to a SCX-2 cartridge which has been pre-eluted with MeOH. The cartridge is eluted with MeOH and then 7M NH₃ in MeOH. Upon standing, a pale yellow solid crystallizes out of the NH₃ in MeOH solution. The solid is collected by filtration, washed with MeOH (20 ml) and dried in vacuo at 40° C. to afford the title compound. [M+H]⁺446.

### Example 27

### 3,5-Diamino-6-chloro-pyrazine-2-carboxylic acid [(S)-4-(hydroxyl-2,2-dimethyl-butyl)-imidazolidin-(2Z)-ylidene]-amide

To a suspension of 3,5-Diamino-6-chloro-pyrazine-2-carboyxlic acid [(S)-4-(4-benzyloxy-2,2-dimethyl-butyl-imidazolidin-(2Z)-ylidene]-amide (Ex. 26) (100 mg, 0.22 mmol) in DCM (5 ml) is added dropwise iodotrimethylsilane (0.061 ml, 0.448 mmol). The resulting yellow solution is heated at reflux for 2 days. The reaction is allowed to cool to room temperature and the yellow solid that has formed is collected by filtration, dissolved in MeOH (3 ml) and loaded onto a 10 g SCX-2 cartridge which has been pre-eluted with MeOH. The cartridge is eluted with MeOH (30 ml) and 7M NH₃ in MeOH (30 ml). The pale yellow 7M NH₃ in MeOH wash is concentrated in vacuo to afford the title compound as a yellow solid. [M+H]⁺356.

### Example 28

### 3,5-Diamino-6-chloro-pyrazine-2-carboxylic acid [(S)-4-{4-[4-(S)-2,3-dihydroxy-propoxy)-phenyl]-2,2-dimethyl-butyl}-imidazolidin-(2Z)-ylidene]-amide

### Step 1

(S)-Glycidol (0.36 ml, 5.5 mmol) is added to a solution of 4-iodophenol (1 g, 4.5 mmol) and triethylamine (31 ml, 0.2 mmol) in ethanol (5 ml) and the resulting light brown solution is heated at reflux for 15 hours. The reaction is allowed to cool to room temperature and the solvent removed in vacuo. The residue is purified by chromatography (SiO₂, EtOAc/iso-hexane) to afford (S)-3-(4-Iodo-phenoxy)-propane-1,2-diol as a colorless oil.

### Step 2

2,2-Dimethoxypropane (1.94 ml, 15.8 mmol) and PPTS (0.079 mg, 0.32 mmol) are added to a solution of (S)-3-(4-Iodo-phenoxy)-propane-1,2-diol (0.93 g, 3.16 mmol) in DMF (20 ml), and the resulting solution is left to stir at room temperature overnight. The solvent is removed in vacuo and the residue is purified by chromatography (SiO₂, EtOAc:Iso-hexane) to afford (R)-4-(4-Iodo-phenoxymethyl)-2,2-dimethyl-[1,3]dioxolane as a colorless oil.

### Step 3

DEAD (0.63 ml, 4 mmol) is added to a suspension of ((S)-1-Hydroxymethyl-3,3-dimethyl-pent-4-enyl)-carbamic acid tert-butyl ester (1 g, 4 mmol), phthalimide (588 mg, 4 mmol) and PS-triphenylphosphine (3.72 g, 8 mmol) in THF (50 ml) and the resulting solution is stirred at room temperature overnight. The resin is removed by filtration, and the filtrate concentrated in vacuo. Purification by flash chromatography (SiO₂, EtOAc/iso-hexane) yields [(S)-1-(1,3-Dioxo-1,3-dihydro-isoindol-2-ylmethyl)-3,3-dimethyl-pent-4-enyl]-carbamic acid tert-butyl ester as a white solid; [M+H−BOC]⁺273.

### Step 4

9-BBN (4.63 ml of a 0.5 M solution in THF, 0.23 mmol) is added to a solution of [(S)-1-(1,3-Dioxo-1,3-dihydro-isoindol-2-ylmethyl)-3,3-dimethyl-pent-4-enyl]-carbamic acid tert-butyl ester (0.43 g, 0.116 mmol) in THF (15 ml) and the resulting colorless solution is stirred at room temperature overnight. Anhydrous DMF (15 ml) is added to the solution, followed by 3 M aqueous K₃PO₄ solution (0.77 ml, 2.3 mmol), (R)-4-(4-Iodo-phenoxymethyl)-2,2-dimethyl-[1,3]dioxolane (267 mg, 0.28 mmol) and Pd(dppf)Cl₂.DCM (47 mg, 0.058 mmol). The reaction is stirred at room temperature for 3 hours, 50° C. for 2 hours and then is allowed to cool to room temperature and filtered through a pad of Celite™ (filter material) which is washed with EtOAc (3×50 ml). The combined filtrates are washed with sat. aq. NaHCO₃ solution (30 ml), dried (MgSO₄) and the solvent removed in vacuo to afford a black oil. Multiple chromatography (SiO₂, EtOAc/iso-hexane) yields [(S)-5-[4-((R)-2,2-Dimethyl-[1,3]dioxolan-4-ylmethoxy)-phenyl]-1-(1,3-dioxo-1,3-dihydro-isoindol-2-ylmethyl)-3,3-dimethyl-pentyl]-carbamic acid tert-butyl ester as a cream solid; [M+H−BOC]⁺481.

### Step 5

Hydrazine (2.2 ml of a 1M solution in THF, 2.2 mmol) is added to a solution of [(S)-5-[4-((R)-2,2-Dimethyl-[1,3]dioxolan-4-ylmethoxy)-phenyl]-1-(1,3-dioxo-1,3-dihydro-isoindol-2-ylmethyl)-3,3-dimethyl-pentyl]-carbamic acid tert-butyl ester (0.16 g, 0.28 mmol) in ethanol (5 ml), and the resulting colorless solution is heated at 45° C. overnight. The reaction is allowed to cool to room temperature, and diethyl ether (30 ml) is added and the resulting white suspension cooled at 0° C. for 30 minutes. The white solid is removed by filtration, and the solvent removed in vacuo to yield {(S)-1-Aminomethyl-5-[4-((R)-2,2-dimethyl-[1,3]dioxolan-4-ylmethoxy)-phenyl]-3,3-dimethyl-pentyl]-carbamic acid tert-butyl ester as a colorless oil; [M+H]⁺451.

### Step 6

A solution of {(S)-1-Aminomethyl-5-[4-((R)-2,2-dimethyl-[1,3]-dioxolan-4-ylmethoxy)-phenyl]-3,3-dimethyl-pentyl}carbamic acid tert-butyl ester (0.13 g, 0.28 mmol) and TFA (1 ml) in DCM (5 ml) is stirred at room temperature for 1 hour, then loaded onto an SCX-2 cartridge which has been pre-eluted with MeOH. The cartridge is eluted with MeOH (2×5 ml), followed by 7M NH₃ in MeOH (2×5 ml) to yield (S)-3-[4-((S)-5,6-Diamino-3,3-dimethyl-hexyl)-phenoxy]-propane-1,2-diol in 80% purity as a colorless oil; [M+H]⁺311.

### Step 7

A suspension of (S)-3-[4-((S)-5,6-Diamino-3,3-dimethyl-hexyl)-phenoxy]-propane-1,2-diol (60 mg, 0.19 mmol) and 1-(3,5-diamino-6-chloro-pyrazine-2-carbonyl)-2-methylisothiourea (Intermediate A) (72 mg, 0.19 mmol) in propan-2-ol (3 ml) is heated at 80° C. for 35 minutes. The reaction mixture is allowed to cool to room temperature and diluted with MeOH until any solid dissolves. The solution is passed through a SCX-2 cartridge which is then eluted with further MeOH. The combined methanol elutions are concentrated in vacuo. Reverse phase chromatography (Isolute™ C18, Water/CH₃CN/0.1% TFA) yields the title compound as a yellow solid; [M+H]⁺506.

### Example 29

### (E)-3,5-diamino-6-chloro-N-(4-(3-(4-((S)-2,3-dihydroxypropoxy)phenyl)propyl)-5-propylimidazolidin-2-ylidene)pyrazine-2-carboxamide hydrochloride

### Step 1

4-(4-Methoxyphenyl)butyric acid (25 g, 129 mmol) is dissolved in 48% HBr (125 ml) and AcOH (125 ml). The resultant solution is heated at 150° C. overnight. The resultant mixture is concentrated in vacuo and the residue taken up in EtOAc (500 ml). This solution is washed with water (500 ml), dried (MgSO₄) and concentrated to give 4-(4-Hydroxy-phenyl)-butyric acid as a tan solid; ¹H NMR (d6-DMSO): 1.72 (2H, tt, J=7.4 and 7.8 Hz), 2.18 (2H, t, J=7.4 Hz), 2.45 (2H, t, J=7.8 Hz), 6.66 (2H, dd, J=1.98 and 9.3 Hz), 6.96 (2H, dd, J=2.8 and 9.3 Hz), 9.12 (1H, s), 12.0 (1H, s).

### Step 2

4-(4-Hydroxy-phenyl)-butyric acid (22.1 g, 123 mmol) is dissolved in THF (750 ml) and borane-dimethyl sulfide (23.3 ml, 245 mmol) is slowly added. The yellow suspension formed is heated at reflux for 3 hours until most of the solid slowly dissolves. The flask is removed from the heating mantle, and MeOH is slowly added until bubbling ceases and the residual solid has dissolved. The flask is cooled to room temperature and water (1 L) is added. The pH is corrected to 3 with AcOH, then the mixture is extracted with EtOAc (2×500 ml). The organics are washed with brine, dried (MgSO₄) and concentrated. The crude product is slurried with silica (500 g) in 25% EtOAc/iso-hexanes (1 L). This is filtered, then flushed with 50% EtOAc/iso-hexanes (2 L) to elute the product. The organics are concentrated to give 4-(4-Hydroxy-butyl)-phenol as a brown oil which crystallizes on standing; ¹H NMR (CDCl₃): 1.55-1.72 (4H, m), 2.58 (2H, t, J=7.0 Hz), 3.1 (2H, br signal), 3.70 (2H, t, J=6.4 Hz), 6.77 (2H, d, J=8.4 Hz), 7.05 (2H, d, J=8.4 Hz).

### Step 3

To 4-(4-Hydroxy-butyl)-phenol (32.7 g, 197 mmol) in acetone (600 ml) is added potassium carbonate (40.8 g, 295 mmol) followed by (S)-glycidol (13.7 ml, 207 mmol). The mixture is heated at reflux overnight. Further potassium carbonate (20 g) is added, followed by (S)-glycidol (5 g) and the mixture is heated at reflux for 72 hours. The suspension is cooled, filtered and the filtrate concentrated in vacuo. The residue is partitioned between EtOAc (500 ml) and 5% citric acid solution (500 ml). The organics are separated, dried (MgSO₄) and concentrated in vacuo to give (S)-3-[4-(4-Hydroxy-butyl)-phenoxy]-propane-1,2-diol as a brown oil; ¹H NMR (CDCl₃): 1.56-1.74 (4H, m), 2.20 (1H, t, J=2.46 Hz), 2.61 (2H, t, J=7.6 Hz), 3.68 (2H, t, J=6.2 Hz), 3.78 (1H, dd, J=5.4 and 11.5 Hz), 3.86 (1H, dd, J=3.9 and 11.5 Hz), 4.0-4.16 (3H, m), 6.85 (2H, d, J=8.6 Hz), 7.12 (2H, d, J=8.6 Hz).

### Step 4

To (S)-3-[4-(4-Hydroxy-butyl)-phenoxy]-propane-1,2-diol (43 g, 179 mmol) in THF (700 ml) is added 2,2-dimethoxypropane (94 ml, 760 mmol) followed by PPTS (4.5 g, 17.9 mmol). The resultant mixture is stirred at room temperature overnight. The solution is concentrated in vacuo and the residue taken up in DCM (500 ml). This is washed with water, dried (MgSO₄) and concentrated in vacuo. The residue is purified through a silica plug (300 g) eluting with 10% followed by 25% EtOAc/iso-hexanes. The desired fractions are concentrated to give 4-[4-((R)-2,2-Dimethyl-[1,3]dioxolan-4-ylmethoxy)-phenyl]-butan-1-ol as a clear oil; ¹H NMR (CDCl₃): 1.42, (3H, s), 1.48 (3H, s), 1.53-1.73 (4H, m), 2.20 (1H, t, J=2.5 Hz), 2.60 (2H, t, J=7.2 Hz), 3.68 (2H, t, J=6.4 Hz), 3.92 (2H, dt, J=5.8 and 8.5 Hz), 4.07 (1H, dd, J=5.4 and 9.5 Hz), 4.19 (1H, dd, J=6.4 and 8.5 Hz), 4.49 (1H, p, J=5.7 Hz), 6.85 (2H, d, J=8.7 Hz), 7.11 (2H, d, J=8.7 Hz).

### Step 5

To 4-[4-((R)-2,2-Dimethyl-[1,3]dioxolan-4-ylmethoxy)-phenyl]-butan-1-ol (5.0 g, 17.8 mmol) in DCM (180 ml) is added Dess-Martin periodinane (7.56 g, 17.8 mmol). The yellowish solution is stirred at room temperature for 1 hour. The resultant yellow suspension is treated with 1N NaOH solution (200 ml) and stirred at room temperature for 30 minutes. The organic phase is separated, dried (MgSO₄) and concentrated to give 4-[4-((R)-2,2-Dimethyl-[1,3]dioxolan-4-ylmethoxy)-phenyl]-butyraldehyde as a clear oil; ¹H NMR (CDCl₃): 1.42, (3H, s), 1.48 (3H, s), 1.95 (2H, dt, J=7.6 and 14.2 Hz), 2.46 (2H, dt, J=1.5 and 7.3 Hz), 2.62 (2H, t, J=7.6 Hz), 3.90-3.96 (2H, m), 4.07 (1H, dd, J=5.2 and 9.3 Hz), 4.19 (1H, dd, J=6.4 and 8.1 Hz), 4.49 (1H, p, J=5.8 Hz), 6.86 (2H, d, J=9.4 Hz), 7.10 (2H, d, J=9.4 Hz), 9.77 (1H, t, J=1.6 Hz).

### Step 6

To 4-[4-((R)-2,2-Dimethyl-[1,3]dioxolan-4-ylmethoxy)-phenyl]-butyraldehyde (4.28 g, 15.4 mmol) in THF (150 ml) is added tert-butyl sulfinamide (2.05 g, 16.9 mmol) followed by titanium ethoxide (6.5 ml, 30.8 mmol). The yellow solution formed is stirred at room temperature overnight. The solution is quenched with 1N NaOH (200 ml) and EtOAc (100 ml) and stirred for 30 minutes at room temperature. The resultant mixture is filtered through Celite™ (filter material) and the organic phase is separated and dried (MgSO₄). Concentration in vacuo gives 2-Methyl-propane-2-sulfinic acid [4-[4-((R)-2,2-dimethyl-[1,3]dioxolan-4-ylmethoxy)-phenyl]-but-(E)-ylidene]-amide as a yellow oil; [M+H]⁺382.23.

### Step 7

To a solution of 2-Methyl-propane-2-sulfnic acid [4-[4-((R)-2,2-dimethyl-[1,3]dioxolan-4-ylmethoxy)-phenyl]-but-(E)-ylidene]-amide (4.51 g, 11.8 mmol) in THF (120 ml) at 0° C. is added vinylmagnesium bromide (11.8 ml of a 1 M solution in THF, 11.8 mmol) dropwise. After addition is complete, the mixture is stirred at 0° C. for 30 minutes then quenched with sat. aq. NH₄Cl solution (20 ml). This mixture is allowed to warm to room temperature and diluted with water (50 ml) and EtOAc (50 ml). The organic phase is separated, dried (MgSO₄) and concentrated in vacuo. Purification by chromatography (SiO₂, EtOAc/iso-hexane) affords 2-Methyl-propane-2-sulfinic acid {4-[4-((R)-2,2-dimethyl-[1,3]dioxolan-4-ylmethoxy)-phenyl]-1-vinyl-butyl}-amide as a mixture of diastereomers as a gum; [M+H]⁺410.39.

### Step 8

A solution of 2-methyl-propane-2-sulfinic acid {4-[4-((R)-2,2-dimethyl-[1,3]dioxolan-4-ylmethoxy)-phenyl]-1-vinyl-butyl}-amide (1.0 g, 2.4 mmol) in DCM (25 ml) at −78° C. is saturated with oxygen, then ozone (generated using Fischer Technology Ozon Generator 500 m) until a blue solution is obtained. Dimethyl sulfide (1.8 ml, 24 mmol) is then added and the mixture stirred to room temperature over 30 minutes. The resultant solution is washed with water (25 ml) and the organic phase is concentrated under high vacuum at low temperature to give 2-Methyl-propane-2-sulfinic acid {4-[4-((R)-2,2-dimethyl-[1,3]dioxolan-4-ylmethoxy)-phenyl]-1-formyl-butyl}-amide as an oil; [M+H]⁺412.36.

### Step 9

To a solution of 2-methyl-propane-2-sulfinic acid {4-[4-((R)-2,2-dimethyl-[1,3]dioxolan-4-ylmethoxy)-phenyl]-1-formyl-butyl}-amide in THF (20 ml) is added tert-butyl sulfinamide (323 mg, 2.7 mmol) followed by titanium ethoxide (1.0 ml, 4.8 mmol). The yellow solution formed is stirred at room temperature overnight. The solution is quenched with 1N NaOH (50 ml) and EtOAc (50 ml) and stirred for 30 minutes at room temperature. The resultant mixture is filtered through Celite™ (filter material) and the organic phase separated and dried (MgSO₄). Concentration gives 2-Methyl-propane-2-sulfnic acid (4-[4-((R)-2,2-dimethyl-[1,3]dioxolan-4-ylmethoxy)-phenyl]-1-{[(E)-2-methyl-propane-2-sulfinylimino]-methyl}-butyl)-amide as a mixture of diastereomers as a yellow oil; [M+H]⁺515.38.

### Step 10

To a solution of 2-methyl-propane-2-sulfnic acid (4-[4-((R)-2,2-dimethyl-[1,3]dioxolan-4-ylmethoxy)-phenyl]-1-{[(E)-2-methyl-propane-2-sulfinylimino]-methyl}-butyl)-amide (907 mg, 1.7 mmol) in THF (20 ml) at 0° C. n-propylmagnesium chloride (1.76 ml of a 2M solution in diethyl ether, 3.52 mmol). The solution is stirred at 0° C. for 30 minutes then at room temperature for 3 hours. A further portion of n-propylmagnesium (1.76 ml of a 2M solution in diethyl ether, 3.52 mmol) is added and the mixture is stirred at room temperature overnight. The resulting mixture is quenched with sat. aq. NH₄C₁ solution (50 ml) and extracted with EtOAc (2×50 ml). The organic phase is dried (MgSO₄) and concentrated in vacuo. The residue is dissolved in EtOAc (10 ml) and treated with 4M HCl/dioxan (10 ml). After 10 minutes, the solution is concentrated in vacuo and the residue diluted with DCM (100 ml). This is treated with sat. aq. NaHCO₃ solution (100 ml) and the organic phase is removed and dried (MgSO₄). The DCM solution is applied to a SCX-2 cartridge (10 g) and this is eluted with DCM and MeOH. The product is released with 2M NH₃ in MeOH, and the methanolic ammonia fraction concentrated to give (S)-3-[4-(4,5-Diamino-octyl)-phenoxy]-propane-1,2-diol as a mixture of diastereomers as a gum; [M+H]⁺515.38.

### Step 11

To a solution of (S)-3-[4-(4,5-Diamino-octyl)-phenoxy]-propane-1,2-diol (100 mg, 0.32 mmol) in propan-2-ol (5 ml) is added 1-(3,5-diamino-6-chloro-pyrazine-2-carbonyl)-2-methyl-isothiourea (Intermediate A) (121 mg, 0.32 mmol). The resulting suspension is heated at 90° C. for 2 hours then cooled and concentrated in vacuo. The residue is dissolved in MeOH (20 ml) and applied to a 10 g SCX-2 cartridge. This is washed well with MeOH, water and MeCN, and then 2M NH₃ in MeOH. The methanolic ammonia fraction is concentrated then purified by chromatography (SiO₂, 5-10% 2M NH₃ in MeOH/DCM). Concentration of the relevant fractions gives the free base as a gum. This is dissolved in MeOH (10 ml) and treated with 1M HCl in diethyl ether (2 ml). Concentration yields the dihydrochloride salt of (E)-3,5-diamino-6-chloro-N-(4-(3-(4-((S)-2,3-dihydroxypropoxy)phenyl)propyl)-5-propylimidazolidin-2-ylidene)pyrazine-2-carboxamide as a yellow solid; [M+H]⁺506.37, 508.36 for Cl isotopes.

### Example 30

### (3-{(S)-2-[(E)-3,5-Diamino-6-chloro-pyrazine-2-carbonylimino]-imidazolidin-4-yl}-propyl)-carbamic acid benzyl ester

1-(3,5-Diamino-6-chloro-pyrazine-2-carbonyl)-2-methyl-isothiourea (Intermediate A) (0.97 g, 3.72 mmol) is stirred in a three necked round bottom flask fitted with a bleach trap and condenser and ((S)-4,5-Diamino-pentyl)-carbamic acid benzyl ester (Intermediate S) (0.85 g, 3.38 mmol) in propan-2-ol (20 ml) is added. The reaction mixture is stirred at 85° C. for 66 hours. Purification by catch and release resin (SCX-2) followed by elution through a silica pad flushed with EtOAc, ethanol and MeOH yields the title compound as an orange foam; [M+H]⁺447.1.

### Example 31

### 3,5-Diamino-6-chloro-pyrazine-2-carboxylic acid [(S)-4-(3-amino-propyl)-imidazolidin-(2E)-ylidene]-amide

To a solution of (3-{(S)-2-[(E)-3,5-Diamino-6-chloro-pyrazine-2-carbonylimino]-imidazolidin-4-yl}-propyl)-carbamic acid benzyl ester (Ex. 30) (0.44 g, 0.98 mmol) in DCM (20 ml) is added iodotrimethylsilane (0.27 ml, 1.96 mmol) in a dropwise manner. The orange suspension is stirred at room temperature for 65 minutes. Purification by catch and release resin (SCX-2) eluting with MeOH followed by 7M NH₃ in MeOH yields the title compound as a yellow foam; [M+H]⁺313.1.

### Example 32

### 3,5-Diamino-6-chloro-pyrazine-2-carboxylic acid [(S)-4-[3-(3-benzyl-ureido)-propyl]-imidazolidin-(2E)-ylidene]-amide

To a solution of 3,5-Diamino-6-chloro-pyrazine-2-carboxylic acid [(S)-4-(3-amino-propyl)-imidazolidin-(2E)-ylidene]-amide (Ex. 31) (0.040 g, 0.128 mmol) in DMF (2 ml) is added 1,1′-carbonyldiimidazole (0.023 g, 0.141 mmol) and the reaction mixture is stirred for 1 hour at room temperature. Benzylamine (0.014 ml, 0.128 mmol) is added and additional benzylamine (0.014 ml, 0.128 mmol) is added at hourly intervals for a total of 3 hours. Purification is by diluting the reaction with 2N NaOH (30 ml) and extracting the product into EtOAc (40 ml). The organic phase is washed with 2N NaOH (30 ml), dried over MgSO₄ and the solvent evaporated in vacuo to yield a yellow oil. The oil is dissolved in methanol (0.75 ml) and diethyl ether (5 ml) added to triturate a yellow solid. This solid is filtered off and the filtrate formed a further precipitate. This yellow solid is collected by filtration and rinsed with diethyl ether to give the title compound; [M+H]⁺446.1.

### Example 33

### 3,5-Diamino-6-chloro-pyrazine-2-carboxylic acid [(S)-4-(3-phenyl methanesulfonylamino-propyl)-imidazolidin-(2E)-ylidene]-amide

To a solution of 3,5-Diamino-6-chloro-pyrazine-2-carboxylic acid [(S)-4-(3-amino-propyl)-imidazolidin-(2E)-ylidene]-amide (Ex. 31) (0.030 g, 0.096 mmol) in DMF (5 ml) at 5° C. is added alpha-toluenesulfonyl chloride (0.018 g, 0.096 mmol) and triethylamine (0.013 ml, 0.096 mmol). The solution is stirred for 10 minutes. Purification by reverse phase chromatography (Isolute™ C18, 0-100% MeCN in water −0.1% TFA) to affords the title compound as a yellow solid; [M+H]⁺467.0.

### Example 34

### 3,5-Diamino-6-chloro-pyrazine-2-carboxylic acid [(S)-4-(3-phenylacetylamino-propyl)-imidazolidin-(2E)-ylidene]-amide

To a solution of 3,5-Diamino-6-chloro-pyrazine-2-carboxylic acid [(S)-4-(3-amino-propyl)-imidazolidin-(2E)-ylidene]-amide (Ex. 31) (0.030 g, 0.96 mmol) in DMF (2 ml), phenylacetyl chloride (0.013 ml, 0.096 mmol) is added. The yellow solution is stirred at room temperature for 10 minutes. Purification by catch and release resin (SCX-2) eluting with MeOH and 7M NH₃ in MeOH affords the title compound; [M+H]⁺430.98.

### Example 35

### 3,5-Diamino-6-chloro-pyrazine-2-carboxylic acid [(S)-4-(4-phenylmethanesulfonylamino-butyl)-imidazolidin-(2E)-ylidene]-amide

To a suspension of 3,5-Diamino-6-chloro-pyrazine-2-carboxylic acid [(S)-4-(4-amino-butyl)-imidazolidin-(2E)-ylidene]-amide (Intermediate T) (0.023 g, 0.071 mmol) in DMF (2 ml) is added triethylamine (0.010 ml, 0.071 mmol) followed by alpha-toluenesulfonyl chloride (0.014 g, 0.071 mmol). The suspension is stirred at room temperature for 30 minutes. Purification by reverse phase chromatography (Isolute™ C18, 0-100% MeCN in water −0.1% TFA) followed by catch and release resin (SCX-2) eluting with MeOH and 7M NH₃ in MeOH gives the title compound as a yellow solid; [M+H]⁺481.0.

### Example 36

### 3,5-Diamino-6-chloro-pyrazine-2-carboxylic acid [(S)-4-(4-phenylacetyl amino-butyl)-imidazolidin-(2E)-ylidene]-amide

To a suspension of 3,5-Diamino-6-chloro-pyrazine-2-carboxylic acid [(S)-4-(4-amino-butyl)-imidazolidin-(2E)-ylidene]-amide (Intermediate T) (0.032 g, 0.098 mmol) in DMF (1 ml) is added triethylamine (0.014 ml, 0.098 mmol) followed by phenylacetyl chloride (0.013 ml, 0.098 mmol). The suspension is stirred at room temperature for 90 minutes before a further 0.5 equivalents of phenylacetyl chloride (0.006 ml, 0.049 mmol) is added. The reaction is left to stir at room temperature for a further 18 hours. Purification by reverse phase chromatography (Isolute™ C18, 0-100% MeCN in water −0.1% TFA) followed by catch and release resin (SCX-2) eluting with MeOH and 7M NH₃ in MeOH affords the title compound as an off-white solid; [M+H]⁺445.1.

### Example 37

### 2-[(E)-3,5-Diamino-6-chloro-pyrazine-2-carbonylimino]-1,3,8-triaza-spiro[4.5]decane-8-carboxylic acid tert-butyl ester trifluoroacetate

A suspension of 4-amino-4-aminomethyl-piperidine-1-carboxylic acid tert-butyl ester (Intermediate U) (218 g, 0.95 mol) in tert-butanol (6 L) and 1-(3,5-diamino-6-chloro-pyrazine-2-carbonyl)-2-methyl-isothiourea (Intermediate A) (338 g, 0.82 mol) is stirred at 40° C. overnight. The temperature is then raised to 85° C. and the suspension stirred at this temperature for a further 4 days. The reaction mixture is concentrated in vacuo and the residue is taken up in water (1 L), sonicated and heated to 45-50° C. The solid is collected by vacuum filtration and washed with ice cold water, then dried under vacuum at 50° C. overnight to afford the title compound as a yellow solid; [M+H]⁺425.1.

### Example 38

### 3,5-Diamino-6-chloro-pyrazine-2-carboxylic acid [1,3,8-triaza-spiro[4.5]dec-(2E)-ylidene]-amide dihydrochloride

To a stirred solution of 4M HCl in dioxane (1 L) is added 2-[(E)-3,5-diamino-6-chloro-pyrazine-2-carbonylimino]-1,3,8-triaza-spiro[4,5]decane-8-carboxylic acid tert-butyl ester trifluoroacetate (Ex. 37) (104 g, 193 mmol). The resulting thick suspension is stirred at room temperature for 2 hours. The product is isolated by vacuum filtration, rinsing with dioxane. The solid is dried under vacuum at 50° C. to afford the title compound as a dihydrochloride salt as a dark yellow solid; [M+H]⁺=325.

### Example 39

### 3,5-Diamino-6-chloro-pyrazine-2-carboxylic acid [8-[(S)-3-phenyl-2-(toluene-4-sulfonylamino)-propionyl]-1,3,8-triaza-spiro[4.5]dec-(2E)-ylidene]-amide

To a solution of Tosyl-L-phenylalanine (1.0 g, 3.13 mmol) in DMF (25 ml) is added N-methyl morpholine (1.033 ml, 9.39 mmol) and 3,5-Diamino-6-chloro-pyrazine-2-carboxylic acid [1,3,8-triaza-spiro[4.5]dec-(2E)-ylidene]-amide dihydrochloride (Ex. 38) (1.37 g, 3.44 mmol), followed by HATU (1.31 g, 3.44 mmol) and the resulting solution is stirred at room temperature for 20 minutes. The crude product is diluted with water (300 ml) and the resultant solid is isolated. Purification by reverse phase chromatography (Isolute™ C18, 0-100% MeCN in water −0.1% TFA) followed by catch and release resin (SCX-2) eluted with MeOH and 7M NH₃ in MeOH yields a yellow solid which is triturated with MeOH and diethyl ether to give the title compound as a free base. The free base is stirred in 5M HCl at 100° C. for 30 minutes forming a gum. MeOH (5 ml) is added to the gum and then all solvent is removed in vacuo. The residue is triturated with MeOH and diethyl ether to give the title compound; [M+H]⁺626.4; ¹H NMR (DMSO-d6): 1.12-1.71 (4H, m), 2.36-2.38 (3H, s), 2.59-2.83 (2H, m), 2.93-3.52 (4H, m), 3.41-3.60 (2H, m), 4.42 (1H, m), 7.12 (2H, d, J=6.9 Hz), 7.17-7.28 (3H, m), 7.35 (2H, d, J=7.7 Hz), 7.54-7.37 (2H, br), 7.57 (2H, d, J=7.7 Hz), 8.12 (1H, d, J=9.0 Hz), 7.70-8.26 (2H, br), 9.22 (1H, s), 9.95 (1H, s), 10.99 (1H, s).

### Example 40

### 3,5-Diamino-6-chloro-pyrazine-2-carboxylic acid [8-(1-benzenesulfonyl-1H-indole-3-carbonyl)-1,3,8-triaza-spiro[4.5]dec-(2E)-ylidene]-amide

To a solution of 1-(phenylsulfonyl)-1H-indole-3-carboxylic acid (1.0 g, 3.32 mmol) in DMF (15 ml) is added HATU (1.388 g, 3.65 mmol) and N-methyl morpholine (1.095 ml, 9.96 mmol) and the solution is stirred at room temperature for 5 minutes. 3,5-Diamino-6-chloro-pyrazine-2-carboxylic acid [1,3,8-triaza-spiro[4.5]dec-(2E)-ylidene]-amide dihydrochloride (Ex. 38) (1.452 g, 3.65 mmol) is added and the reaction stirred at room temperature for 45 minutes. The reaction mixture is diluted with water (100 ml) and the precipitate that forms is isolated by filtration. The crude product is suspended in 2N NaOH and extracted into EtOAc. The organic portion is dried over MgSO₄ and concentrated in vacuo to yield a brown solid. The solid is suspended in a 1:1 mixture of water (+0.1% TFA) and acetonitrile. A fine brown solid is removed by filtration and the yellow filtrate is concentrated in vacuo until 10 ml of solvent remains and a pale yellow solid has precipitated. This solid is washed with 2N NaOH (60 ml) and then suspended in 2N NaOH (100 ml) and extracted into EtOAc (2×100 ml). The organic phases are combined, dried over MgSO₄ and concentrated in vacuo to yield a pale cream solid. The cream solid is suspended in a 1:4 mixture of EtOAc: iso-hexane (100 ml) and the solid filtered off to give the free base of the title compound, which is suspended in 5N HCl (20 ml) and stirred for 2 hours. MeOH (20 ml) is added to dissolve all solid and the solvent is concentrated in vacuo until a yellow solid precipitates. This solid is filtered off, rinsed with water and dried at 40° C. for 18 hours to give the title compound; [M+H]⁺607.42; ¹H NMR (DMSO-d6): 1.86-1.92 (4H, m), 3.42-3.63 (4H, m), 3.68 (2H, s), 7.34 (1H, dd, J+7.5 Hz, J=7.5 Hz), 7.43 (1H, dd, J=7.5 Hz, J=7.5 Hz), 7.36-7.55 (2H, br), 7.62 (1H, d, J=7.5 Hz), 7.63 (2H, m), 7.73 (1H, m), 7.99 (1H, d, J=7.5 Hz), 8.06 (2H, obs), 8.07 (1H, s), 7.50-8.16 (2H, br), 9.18 (1H, s), 9.77 (1H, s), 11.09 (1H, s).

### Example 41

### 3,5-Diamino-6-chloro-pyrazine-2-carboxylic acid [843-(3-isopropoxy-propylsulfamoyl)-benzoyl]-1,3,8-triaza-spiro[4.5]dec-(2E)-ylidene]-amide

To a solution of 3-(3-Isopropoxy-propylsulfamoyl)-benzoic acid (Intermediate V) (1.10 g, 3.65 mmol) in DMF (20 ml) is added HATU (1.53 g, 4.02 mmol) and N-methyl morpholine (1.204 ml, 10.95 mmol) and the solution is stirred at room temperature for 5 minutes. 3,5-Diamino-6-chloro-pyrazine-2-carboxylic acid [1,3,8-triaza-spiro[4.5]dec-(2E)-ylidene]-amide dihydrochloride (Ex. 38) (1.60 g, 4.02 mmol) is added and the reaction stirred at room temperature for 45 minutes. The reaction mixture is diluted with 2N NaOH (150 ml) and the crude product extracted into EtOAc (2×250 ml). The organic phase is dried over MgSO₄ and the solvent evaporated in vacuo to yield a yellow oil. Purification on a Waters preparative Delta 3000 HPLC using a gradient of water (+0.1% TFA) and acetonitrile yields a yellow oil. 2N NaOH is added to the oil and the product is extracted into EtOAc (2×400 ml). The organic phases are combined, dried over MgSO₄ and the solvent concentrated in vacuo to a volume of approximately 150 ml. To this solution is added iso-hexane (400 ml) and a pale yellow solid precipitates. This solid is collected by filtration and rinsed with iso-hexane to afford the title compound; [M+H]⁺607.98; ¹H Nmr (DMSO): 1.00 (6H, d, J=6.0 Hz), 1.55 (2H, m), 1.69-1.79 (4H, m), 2.81 (2H, t, 5.9 Hz), 3.29 (2H, tr, J=6.0 Hz), 3.42 (1H, m), 3.44 (2H, br), 3.29-3.82 (4H, m), 6.15-7.30 (3H, br), 7.66 (1H, d, J=7.4 Hz), 7.70 (1H, dd, J=7.4 Hz, J=7.4 Hz), 7.76 (1H, s), 7.86 (1H, d, J=7.4 Hz), 7.44-8.00 (1H, br), 8.00-9.05 (3H, br).

### Example 42

### 3,5-Diamino-6-chloro-pyrazine-2-carboxylic acid [8-[2-(5-phenyl-4H-[1,2,4]triazol-3-yl)-acetyl]-1,3,8-triaza-spiro[4.5]dec-(2E)-ylidene]-amide

(5-Phenyl-4H[1,2,4]traizol-3-yl)acetic acid (0.48 g, 2.364 mmol), HATU (0.988 g, 2.6 mmol), 5-Diamino-6-chloro-pyrazine-2-carboxylic acid [1,3,8-triaza-spiro[4.5]dec-(2E)-ylidene]-amide dihydrochloride (Example. 38) (1.033 g, 2.60 mmol), DMF (20 ml) and N-methyl morpholine (0.78 ml, 7.08 mmol) are added to a round bottomed flask and stirred at room temperature for 20 minutes. The crude product is precipitated from the reaction mixture by adding water (200 ml) and is isolated by filtration. Purification by reverse phase chromatography (Isolute™ C18, 0-100% MeCN in water −0.1% TFA) yields a yellow semi-solid. This is dissolved in MeOH (100 ml) and left to stand. An off white solid precipitates and this is collected by filtration to give the title compound; [M+H]+510.0; ¹H NMR (DMSO-d6): 1.78-1.94 (4H, m), 3.67 (2H, s), 3.30-3.82 (4H, m), 4.05-4.08 (2H, m), 7.45-7.55 (3H m), 7.01-7.75 (3H, br), 8.05 (2H, d, J=7.1 Hz), 7.78-8.33 (2H, br), 9.24 (1H, s), 9.85 (1H, s), 11.04 (1H, s).

### Example 43

### 3,5-Diamino-6-chloro-pyrazine-2-carboxylic acid [8-[3-(3-isopropyl-ureido)-benzoyl]-1,3,8-triaza-spiro[4.5]dec-(2E)-ylidene]-amide

3-(3-Isopropyl-ureido)-benzoic acid (Intermediate W) (1.08 g, 4.86 mmol) and HATU (2.03 g, 5.35 mmol) are stirred in DMF (25 ml) at room temperature and N-methyl morpholine (1.60 ml, 14.59 mmol) is added. The solution is stirred at room temperature for 5 minutes and 5-Diamino-6-chloro-pyrazine-2-carboxylic acid [1,3,8-triaza-spiro[4.5]dec-(2E)-ylidene]-amide dihydrochloride (Example 38) (2.13 g, 5.35 mmol) is added. The brown solution is stirred at room temperature for 45 minutes. The crude product is precipitated by the addition of 2N NaOH and collected by filtration. The solid is purified by reverse phase chromatography (Isolute™ C18, 0-100% MeCN in water −0.1% TFA). The clean fractions are concentrated in vacuo to approximately 30 ml and 2N NaOH added. The off white solid is collected by filtration and rinsed with water to give the title compound; [M+H]+529.05; ¹H NMR (DMSO-d6): 1.09 (6H, d, J=6.5 Hz), 1.67-1.73 (4H, m), 3.42 (2H, br), 3.75 (1H, septet, J=6.5 Hz), 3.31-3.79 (4H, br), 6.15 (1H, d, J=7.5 Hz), 6.70 (2H, br), 6.40-7.01 (1H, br), 6.86 (1H, d, J=7.2 Hz), 7.26 (1H, dd, J=8.3 Hz, J=7.2 Hz), 7.31 (1H, d, J=8.3 Hz), 7.53 (1H, s), 8.36 (1H, br), 8.48 (1H, br), 8.55 (1H, s), 8.00-9.00 (1H, br).

### Example 44

### 3,5-Diamino-6-chloro-pyrazine-2-carboxylic acid [8-(2-Benzo[b]thiophen-3-yl-acetyl)-1,3,8-triaza-spiro[4.5]dec-(2E)-ylidene]-Amide

This compound is prepared analogously to Example 43 by replacing 3-(3-Isopropyl-ureido)-benzoic acid (Intermediate W) with benzo[b]thiophene-3-acetic acid. [M−H]⁺499.0; ¹H NMR (DMSO-d6): 1.59-1.74 (4H, m), 3.42 (2H, s), 3.48-3.95 (4H, m), 3.97 (2H, s), 6.20-7.11 (3H, br), 7.38 (1H, m), 7.39 (1H, m), 7.50 (1H, s), 7.83 (1H, d, J=7.3 Hz), 7.97 (111, d, J=7.6 Hz), 7.75-9.30 (3H, br).

### Example 45

### 3,5-Diamino-6-chloro-pyrazine-2-carboxylic acid [8-[5-oxo-1-(3-pyrrol-1-yl-propyl)-pyrrolidine-3-carbonyl]-1,3,8-triaza-spiro[4.5]dec-(2E)-ylidene]-amide

A solution of 5-Oxo-1-(3-pyrrol-1-yl-propyl)-pyrrolidine-3-carboxylic acid (Intermediate X) (1.15 g, 4.85 mmol), HATU (2.03 g, 5.33 mmol), DMF (20 ml) and N-methyl morpholine (1.60 ml, 14.54 mmol) is stirred at room temperature for 5 minutes before 5-Diamino-6-chloro-pyrazine-2-carboxylic acid [1,3,8-triaza-spiro[4.5]dec-(2E)-ylidene]-amide dihydrochloride (Ex. 38) (1.731 g, 5.33 mmol) is added. After stirring for 60 minutes at room temperature EtOAc (200 ml) is added and the organic phase is washed with 2N NaOH (2×100 ml) and brine (100 ml). The organic phase is dried over MgSO₄ and the solvent evaporated in vacuo. Purification by reverse phase chromatography (Isolute™ C18, 0-100% MeCN in water −0.1% TFA) followed by catch and release resin (SCX-2) eluting with MeOH and 7M NH₃ in MeOH yields a yellow oil. The oil is dissolved in DCM (10 ml) and product is precipitated out of solution by the addition of iso-hexane to yield a yellow solid which is filtered and rinsed with iso-hexane to yield the title product; [M+H]⁺542.8; ¹H NMR (DMSO-d6): 1.64-1.70 (4H, m), 1.84-1.89 (2H, m), 2.43-2.51 (2H, m), 3.39-3.43 (2H, m), 3.43-3.50 (2H, m), 3.55 (1H, m), 3.40-3.69 (4H, m), 3.84 (2H, m), 5.97 (2H, m), 6.65-6.74 (2H, br), 6.75 (2H, m), 6.2-7.6 (1H, br), 7.6-9.5 (1H, br).

### Example 46

### 3,5-Diamino-6-chloro-pyrazine-2-carboxylic acid [8-(6,7,8,9-tetrahydro-5H-carbazole-3-carbonyl)-1,3,8-triaza-spiro[4.5]dec-(2E)-ylidene]-amide

To a stirring solution of 6,7,8,9-Tetrahydro-5H-carbazole-3-carboxylic acid (0.05 g, 0.25 mmol) and HATU (0.11 g, 0.28 mmol) in dry DMF (5 ml) is added N-methyl morpholine (0.08 ml, 0.76 mmol). After 5 minutes stirring at room temperature, 5-Diamino-6-chloro-pyrazine-2-carboxylic acid [1,3,8-triaza-spiro[4.5]dec-(2E)-ylidene]-amide dihydrochloride (Ex. 38) (0.10 g, 0.28 mmol) is added and the reaction is left to stir at room temperature for 1 hour. Purification by reverse phase chromatography (Isolute™ C18, 0-100% MeCN in water) yields the title compound as a yellow powder; [M+H]⁺524.2.

### Example 47

### 3,5-Diamino-6-chloro-pyrazine-2-carboxylic acid [8-(1H-indazole-3-carbonyl)-1,3,8-triaza-spiro[4.5]dec-(2E)-ylidene]-amide

To a stirring solution of 1H-indazole-3-carboxylic acid (0.041 g, 0.25 mmol) and HATU (0.096 g, 0.25 mmol) in dry DMF (4 ml) is added N-methyl morpholine (0.08 ml, 0.76 mmol). After 5 minutes, 5-Diamino-6-chloro-pyrazine-2-carboxylic acid [1,3,8-triaza-spiro[4.5]dec-(2E)-ylidene]-amide dihydrochloride (Ex. 38) (0.10 g, 0.25 mmol) is added and the reaction left to stir at room temperature for 1 hour. Purification by reverse phase chromatography (Isolute™ C18, 0-100% MeCN in water −0.1% TFA) yields an oily residue that is ultrasonicated in acetonitrile to give a yellow suspension. The yellow solid is collected by filtration and rinsed with acetonitrile to afford the title compound; [M+H]⁺469.17.

### Example 48

### 3,5-Diamino-6-chloro-pyrazine-2-carboxylic acid [8-[2-(2,3-dimethyl-1H-indol-5-yl)-acetyl]-1,3,8-triaza-spiro[4.5]dec-(2E)-ylidene]-amide

This compound is prepared analogously to Example 47 by replacing 1H-indazole-3-carboxylic acid with 2-(2,3-dimethyl-1H-indol-5-yl)acetic acid with 2-(2,3-dimethyl-1H-indol-5-yl)acetic acid. [M+H]⁺510.23.

### Example 49

### 3,5-Diamino-6-chloro-pyrazine-2-carboxylic acid [8-(1,2,3-trimethyl-1H-indole-5-carbonyl)-1,3,8-triaza-spiro[4.5]dec-(2E)-ylidene]-amide

To a stirring solution of 1,2,3-trimethyl-1H-indole-5-carboxylic acid (0.051 g, 0.25 mmol) and HATU (0.11 g, 0.28 mmol) in dry DMF (5 ml) is added N-methyl morpholine (0.083 ml, 0.76 mmol). After 5 minutes 5-Diamino-6-chloro-pyrazine-2-carboxylic acid [1,3,8-triaza-spiro[4.5]dec-(2E)-ylidene]-amide dihydrochloride (Ex. 38) (0.10 g, 0.28 mmol) is added and the reaction left to stir at room temperature for 1 hour. Purification by chromatography (SiO₂, MeOH/DCM) yields the title compound; [M+H]⁺510.1.

### Example 50

### 3,5-Diamino-6-chloro-pyrazine-2-carboxylic acid [8-(1-methyl-1H-indazole-3-carbonyl)-1,3,8-triaza-spiro[4.5]dec-(2E)-ylidene]-amide

This compound is prepared analogously to Example 46 by replacing 6,7,8,9-Tetrahydro-5H-carbazole-3-carboxylic acid with 1-methyl-1H-indazole-3-carboxylic acid. [M+H]⁺483.1.

### Example 51

### 3,5-Diamino-6-chloro-pyrazine-2-carboxylic acid [8-(4-benzyloxy-benzoyl)-1,3,8-triaza-spiro[4.5]dec-(2E)-ylidene]-amide

5-Diamino-6-chloro-pyrazine-2-carboxylic acid [1,3,8-triaza-spiro[4.5]dec-(2E)-ylidene]-amide dihydrochloride (Example 38) (0.05 g, 0.13 mmol), 4-(benzyloxy)benzoic acid (0.029 g, 0.13 mmol), HATU (0.05 g, 0.13 mmol), N-methyl morpholine (0.041 ml, 0.38 mmol) and DMF (2 ml) are stirred together at room temperature for 72 hours. The reaction mixture is diluted with EtOAc (25 ml) and washed with water (25 ml) and sat. NaHCO₃ (25 ml). The organic phase is dried over MgSO₄ and evaporated in vacuo to yield a yellow oil. The oil is dissolved in ethyl acetate and a drop of methanol and iso-hexane are added. The resulting pale yellow solid is collected by filtration to give the title compound; [M+H]⁺535.1.

### Example 52

### 3,5-Diamino-6-chloro-pyrazine-2-carboxylic acid [8-(3-2,3-dihydro-benzofuran-5-yl-propionyl)-1,3,8-triaza-spiro[4.5]dec-(2E)-ylidene]-amide

This compound is prepared analogously to Example 51 by replacing 4-(benzyloxy)benzoic acid with 3-(2,3-dihydrobenzofuran-5-yl)propanoic acid. [M+H]⁺499.1.

### Example 53

### 3,5-Diamino-6-chloro-pyrazine-2-carboxylic acid [8-(1H-pyrrolo[2,3-b]pyridine-4-carbonyl)-1,3,8-triaza-spiro[4.5]dec-(2E)-ylidene]-amide

This compound is prepared analogously to Example 47 by replacing 1H-indazole-3-carboxylic acid with 1H-pyrrolo[2,3-b]pyridine-4-carboxylic acid; [M+H]⁺469.14.

### Example 54

### 3,5-Diamino-6-chloro-pyrazine-2-carboxylic acid [8-[3-(4-methoxy-phenyl)-propionyl]-1,3,8-triaza-spiro[4.5]dec-(2E)-ylidene]-amide

5-Diamino-6-chloro-pyrazine-2-carboxylic acid [1,3,8-triaza-spiro[4.5]dec-(2E)-ylidene]-amide dihydrochloride (Example 38) (0.05 g, 0.13 mmol), 3-(4-methoxyphenyl)-propionic acid (0.023 g, 0.13 mmol), HATU (0.048 g, 0.13 mmol), N-methyl morpholine (0.041 ml, 0.38 mmol) and DMF (2 ml) are stirred together at room temperature for 48 hours. The reaction mixture is diluted with EtOAc (50 ml) and product is extracted into 1 M HCl. The aqueous phase is basified to pH 12 with 2 N NaOH and product extracted into EtOAc (50 ml). The organic phase is dried over MgSO₄ and the solvent evaporated in vacuo to yield a brown glass. The product is triturated with MeOH and EtOAc to give a pale brown solid as the title compound; [M+H]⁺487.0.

### Example 55

### 3,5-Diamino-6-chloro-pyrazine-2-carboxylic acid [8-[3-(4-hydroxy-phenyl)-propionyl]-1,3,8-triaza-spiro[4.5]dec-(2E)-ylidene]-amide

This compound is prepared analogously to Example 49 by replacing 1,2,3-trimethyl-1H-indole-5-carboxylic acid with 13-(4-hydroxyphenyl)propionic acid; [M+H]⁺472.98.

### Example 56

### 3,5-Diamino-6-chloro-pyrazine-2-carboxylic acid [8-(1H-indole-2-carbonyl)-1,3,8-triaza-spiro[4.5]dec-(2E)-ylidene]-amide

This compound is prepared analogously to Example 46 by replacing 6,7,8,9-Tetrahydro-5H-carbazole-3-carboxylic acid with 1H-indole-2-carboxylic acid; [M+H]⁺468.1.

### Example 57

### 3,5-Diamino-6-chloro-pyrazine-2-carboxylic acid [8-(quinoline-5-carbonyl)-1,3,8-triaza-spiro[4.5]dec-(2E)-ylidene]-amide

This compound is prepared analogously to Example 46 by replacing 6,7,8,9-Tetrahydro-5H-carbazole-3-carboxylic acid with quinoline-5-carboxylic acid; [M+H]⁺480.1.

### Example 58

### 3,5-Diamino-6-chloro-pyrazine-2-carboxylic acid [8-(4-methyl-2-phenyl-pyrimidine-5-carbonyl)-1,3,8-triaza-spiro[4.5]dec-(2E)-ylidene]-amide

This compound is prepared analogously to Example 45 by replacing 5-Oxo-1-(3-pyrrol-1-yl-propyl)-pyrrolidine-3-carboxylic acid (Intermediate X) with 4-methyl-2-phenylpyrimidine-5-carboxylic acid; [M+H]⁺521.1.

### Example 59

### 3,5-Diamino-6-chloro-pyrazine-2-carboxylic acid [8-(4-benzyl-morpholine-2-carbonyl)-1,3,8-triaza-spiro[4.5]dec-(2E)-ylidene]-amide

This compound is prepared analogously to Example 51 by replacing 4-(benzyloxy)benzoic acid with 4-benzyl-2-morpholinecarboxylic acid hydrochloride; [M+H]⁺528.2.

### Example 60

### 3,5-Diamino-6-chloro-pyrazine-2-carboxylic acid [8-(1H-pyrrolo[2,3-b]pyridine-5-carbonyl)-1,3,8-triaza-spiro[4.5]dec-(2E)-ylidene]-amide

This compound is prepared analogously to Example 47 by replacing 1H-indazole-3-carboxylic acid with 1H-pyrrolo[2,3-b]pyridine-5-carboxylic acid; [M+H]⁺469.1.

### Example 61

### 3,5-Diamino-6-chloro-pyrazine-2-carboxylic acid [8-[4-(4,6-dimethoxy-pyrimidin-2-ylmethoxy)-benzoyl]-1,3,8-triaza-spiro[4.5]dec-(2E)-ylidene]-amide

This compound is prepared analogously to Example 51 by replacing 4-(benzyloxy)benzoic acid with 4-((4,6-dimethoxypyrimidin-2-yl)methoxy)benzoic acid; [M+H]⁺597.07.

### Example 62

### 3,5-Diamino-6-chloro-pyrazine-2-carboxylic acid [8-[2-(3-isopropyl-ureido)-pyridine-4-carbonyl]-1,3,8-triaza-spiro[4.5]dec-(2E)-ylidene]-amide

This compound is prepared analogously to Example 51 by replacing 4-(benzyloxy)benzoic acid with 2-(3-Isopropyl-ureido)-isonicotinic acid (intermediate Y) [M+H]⁺530.2; ¹H NMR (DMSO-d6): 1.13 (6H, d, J=6.5), 1.77-1.94 (4H, m), 3.66 (2H, d, J=11), 3.25-3.99 (5H, m), 6.97 (1H, br m), 7.50 (1H, br s), 7.31-7.60 (2H, br s), 7.61 (1H, br s), 7.74-8.25 (2H, br s), 8.28 (1H, d, J=5.5), 9.08-9.21 (1H, br s), 9.60-9.80 (1H, br s), 9.70-10.25 (1H, br s), 11.07 (s, 1H).

### Example 63

### 4-12-[(E)-3,5-Diamino-6-chloro-pyrazine-2-carbonylimino]-1,3,8-triaza-spiro[4.5]decane-8-carbonyl}-indole-1-carboxylic acid isopropylamide

This compound is prepared analogously to Example 51 by replacing 4-(benzyloxy)benzoic acid with 1-isopropylcarbamoyl-1H-indole-4-carboxylic acid (Intermediate Z): [M+H]⁺553.5.

### Example 64

### 3,5-Diamino-6-chloro-pyrazine-2-carboxylic acid [8-[4-(3-isopropyl-ureido)-benzoyl]-1,3,8-triaza-spiro[4.5]dec-(2E)-ylidene]-amide

This compound is prepared analogously to Example 51 by replacing 4-(benzyloxy)benzoic acid with 4-(3-isopropyl-ureido)-benzoic acid (Intermediate AA); [M+H]⁺529.5.

### Example 65

### 3,5-Diamino-6-chloro-pyrazine-2-carboxylic acid [8-[6-(3-isopropyl-ureido)-pyridine-3-carbonyl]-1,3,8-triaza-spiro[4.5]dec-(2E)-ylidene]-amide

This compound is prepared analogously to Example 51 by replacing 4-(benzyloxy)benzoic acid with 6-(3-isopropyl-ureido)-nicotinic acid (Intermediate AB); [M+H]⁺530.5.

### Example 66

### 3,5-Diamino-6-chloro-pyrazine-2-carboxylic acid [8-[3-(4-allyloxy-phenyl)-propionyl]-1,3,8-triaza-spiro[4.5]dec-(2E)-ylidene]-amide

This compound is prepared analogously to Example 51 by replacing 4-(benzyloxy)benzoic acid with 3-(4-allyloxy)phenyl)propanoic acid; [M+H]⁺513.4.

### Example 67

### 3,5-Diamino-6-chloro-pyrazine-2-carboxylic acid [8-{2-[4-(2-methoxy-ethoxymethoxy)-phenyl]-acetyl}-1,3,8-triaza-spiro[4.5]dec-(2E)-ylidene]-amide

This compound is prepared analogously to Example 51 by replacing 4-(benzyloxy)benzoic acid with [4-(2-methoxy-ethoxymethoxy)-phenyl]-acetic acid (Intermediate AC); 547.4.

### Example 68

### 3,5-Diamino-6-chloro-pyrazine-2-carboxylic acid [8-{3-[4-(2-methoxy-ethoxymethoxy)-phenyl}-propionyl}-1,3,8-triaza-spiro[4.5]dec-(2E)-ylidene]-amide

This compound is prepared analogously to Example 2.13 by replacing 4-(benzyloxy)benzoic acid with 3-[4-(2-Methoxy-ethoxymethoxy)-phenyl]-propionic acid (Intermediate AD); [M+H]⁺561.0.

### Example 69

### 3,5-Diamino-6-chloro-pyrazine-2-carboxylic acid [8-(3-{4[2-(tetrahydro-pyran-2yloxy)-ethoxy]-phenyl}-propionyl)-1,3,8-triaza-spiro[4.5]dec-(2E)-ylidene]-amide

This compound is prepared analogously to Example 51 by replacing 4-(benzyloxy)benzoic acid with 3-{4-[2-(tetrahydro-pyran-2-yloxy)-ethoxy]-phenyl}-propionic acid (Intermediate AE); [M+H]⁺601.1.

### Example 70

### 3,5-Diamino-6-chloro-pyrazine-2-carboxylic acid [8-{3-[4-(pyridin-4-ylmethoxy)-phenyl]-propionyl}-1,3,8-triaza-spiro[4.5]dec-(2E)-ylidene]-amide

This compound is prepared analogously to Example 51 by replacing 4-(benzyloxy)benzoic acid with 3[4-(Pyridin-4-ylmethoxy)-phenyl]-propionic acid (Intermediate AF); [M+H]⁺564.1.

### Example 71

### [4-(3-{2-[(E)-3,5-Diamino-6-chloro-pyrazine-2-carbonylimino]-1,3,8-triaza-spiro[4.5]dec-8-yl}-3-oxo-propyl)-phenoxy]-acetic acid tert-butyl

This compound is prepared analogously to Example 51 by replacing 4-(benzyloxy)benzoic acid with 3-(4-tert-butoxycarbonylmethoxy-phenyl)-propionic acid (Intermediate AG); [M+H]⁺587.5.

### Example 72

### 3,5-Diamino-6-chloro-pyrazine-2-carboxylic acid [8-[3-(4-carbamoylmethoxy-phenyl)-propionyl]-1,3,8-triaza-spiro[4.5]dec-(2E)-ylidene]-amide

This compound is prepared analogously to Example 51 by replacing 4-(benzyloxy)benzoic acid with 3-(4-Carbamoylmethoxy-phenyl)-propionic acid (Intermediate AH); [M+H]⁺530.1.

### Example 73

### 1-[4-(3-{2-[(E)-3,5-Diamino-6-chloro-pyrazine-2-carbonylimino]-1,3,8-triaza-spiro[4.5]dec-8-yl}-3-oxo-propyl)-phenoxy]-cyclobutanecarboxylic acid ethyl ester

This compound is prepared analogously to Example 51 by replacing 4-(benzyloxy)benzoic acid with 1-[4-(2-Carboxy-ethyl)-phenoxy]-cyclobutanecarboxylic acid ethyl ester (Intermediate AI); [M+H]⁺599.1.

### Example 74

### 2-[4-(3-{2-[(E)-3,5-Diamino-6-chloro-pyrazine-2-carbonylimino]-1,3,8-triaza-spiro[4.5]dec-8-yl}-3-oxo-propyl)-phenoxy]-2-methyl-propionic acid tert-butyl ester

This compound is prepared analogously to Example 51 by replacing 4-(benzyloxy)benzoic acid with 2-[4-(2-Carboxy-ethyl)-phenoxy]-2-methyl-propionic acid tert-butyl ester (Intermediate AJ); [M+H]⁺615.2.

### Example 75

### [4-(3-{2-[(E)-3,5-Diamino-6-chloro-pyrazine-2-carbonylimino]-1,3,8-triaza-spiro [4.5]dec-8-yl}-3-oxo-propyl)-phenoxy]-acetic acid methyl ester

This compound is prepared analogously to Example 51 by replacing 4-(benzyloxy)benzoic acid with 3-(4-Methoxycarbonylmethoxy-phenyl)-propionic acid (Intermediate AK); [M+H]⁺545.1.

### Example 76

### 4-{2-[(E)-3,5-Diamino-6-chloro-pyrazine-2-carbonylimino]-1,3,8-triaza-spiro[4.5]decane-8-carbonyll-benzoic acid tert-butyl ester

This compound is prepared analogously to Example 51 by replacing 4-(benzyloxy)benzoic acid with 4-(tert-Butoxycarbonyl)benzoic acid; [M+H]⁺529.4.

### Example 77

### 3,5-Diamino-6-chloro-pyrazine-2-carboxylic acid [8-(3-isopropyl-2-methyl-1H-indole-5-carbonyl)-1,3,8-triaza-spiro[4.5]dec-(2E)-ylidene]-amide

This compound is prepared analogously to Example 51 by replacing 4-(benzyloxy)benzoic acid with 3-isopropyl-2-methyl-1H-indole-5-carboxylic acid; [M+H]⁺524.

### Example 78

### 3-[4-(3-{2-[(E)-3,5-Diamino-6-chloro-pyrazine-2-carbonylimino]-1,3,8-triaza-spiro [4.5]dec-8-yl}-3-oxo-propyl)-phenyl]-propionic acid propyl ester

This compound is prepared analogously to Example 51 by replacing 4-(benzyloxy)benzoic acid with 344-(2-Propoxycarbonyl-ethyl)-phenyl]-propionic acid (intermediate AL); [M+H]⁺571.

### Example 79

### 3-[4-(3-{2-[(E)-3,5-Diamino-6-chloro-pyrazine-2-carbonylimino]-1,3,8-triaza-spiro[4.5]dec-8-yl}-3-oxo-propyl)-phenyl]-propionic acid ethyl ester

This compound is prepared analogously to Example 51 by replacing 4-(benzyloxy)benzoic acid with 344-(2-Ethoxycarbonyl-ethyl)-phenyl]-propionic acid (intermediate AM); [M+H]⁺557.

### Example 80

3-[4-(3-{2-[(E)-3,5-Diamino-6-chloro-pyrazine-2-carbonylimino]-1,3,8-triaza-spiro[4.5]dec-8-yl}-3-oxo-propyl)-phenyl]-propionic acid methyl ester

This compound is prepared analogously to Example 51 by replacing 4-(benzyloxy)benzoic acid with 3-[4-(2-Methoxycarbonyl-ethyl)-phenyl]-propionic acid (intermediate AN); [M+H]⁺543.

### Example 81

### 3-[4-(3-{2-[(E)-3,5-Diamino-6-chloro-pyrazine-2-carbonylimino]-1,3,8-triaza-spiro[4.5]dec-8-yl}-3-oxo-propyl)-phenyl]-propionic acid

This compound is prepared analogously to Example 51 by replacing 4-(benzyloxy)benzoic acid with 3,3′-(1,4-phenylene)dipropanoic acid; [M+H]⁺529.

### Example 82

### 3,5-Diamino-6-chloro-pyrazine-2-carboxylic acid [8-[1-(2-phenoxy-ethyl)-1H-indole-4-carbonyl]-1,3,8-triaza-spiro[4.5]dec-(2E)-ylidene]-amide

This compound is prepared analogously to Example 51 by replacing 4-(benzyloxy)benzoic acid with 1-(2-Phenoxy-ethyl)-1H-indole-4-carboxylic acid (Intermediate AO); [M+H]⁺588.

### Example 83

### 3,5-Diamino-6-chloro-pyrazine-2-carboxylic acid [8-[1-(2-p-tolyl-ethyl) 1H-indole-4-carbonyl]-1,3,8-triaza-spiro[4.5]dec-(2E)-ylidene]-amide

This compound is prepared analogously to Example 51 by replacing 4-(benzyloxy)benzoic acid with 1-(2-p-Tolyl-ethyl)-1H-indole-4-carboxylic acid (Intermediate AP); [M+H]⁺586.

### Example 84

### 3,5-Diamino-6-chloro-pyrazine-2-carboxylic acid [8-{1-[2-(tetrahydro-pyran-2-yloxy)-ethyl]-1H-indole-4-carbonyl}-1,3,8-triaza-spiro[4.5]dec-(2E)-ylidene]-amide

This compound is prepared analogously to Example 51 by replacing 4-(benzyloxy)benzoic acid with 1-[2-(Tetrahydro-pyran-2-yloxy)-ethyl]-1H-indole-4-carboxylic acid (Intermediate AQ); [M+H]⁺597.

### Example 85

### 3,5-Diamino-6-chloro-pyrazine-2-carboxylic acid [8-{1-[2-(4-methoxy-phenoxy)-ethyl]-1H-indole-4-carbonyl}-1,3,8-triaza-spiro[4.5]dec-(2E)-ylidene]-amide

This compound is prepared analogously to Example 51 by replacing 4-(benzyloxy)benzoic acid with 1-[2-(4-Methoxy-phenoxy)-ethyl]-1H-indole-4-carboxylic acid (Intermediate AR); [M+H]⁺618.

### Example 86

### 3,5-Diamino-6-chloro-pyrazine-2-carboxylic acid [8-{1-[2-(4-tert-butyl-phenoxy)-ethyl]-1H-indole-4-carbonyl}-1,3,8-triaza-spiro[4.5]dec-(2E)-ylidene]-amide

This compound is prepared analogously to Example 51 by replacing 4-(benzyloxy)benzoic acid with 1-[2-(4-tert-Butyl-phenoxy)-ethyl]-1H-indole-4-carboxylic acid (Intermediate AS); [M+H]⁺644.

### Example 87

### 3,5-Diamino-6-chloro-pyrazine-2-carboxylic acid [8-[1-(2-[1,3]dioxan-2-yl-ethyl)-1H-indole-4-carbonyl]-1,3,8-triaza-spiro[4.5]dec-(2E)-ylidene]-amide

This compound is prepared analogously to Example 51 by replacing 4-(benzyloxy)benzoic acid with 1-(2-[1,3]Dioxan-2-yl-ethyl)-1H-indole-4-carboxylic acid (Intermediate AT; [M+H]⁺582.

### Example 88

### 3,5-Diamino-6-chloro-pyrazine-2-carboxylic acid [8-[1-(2-hydroxy-ethyl)-2,3-dimethyl-1H-indole-5-carbonyl]-1,3,8-triaza-spiro[4.5]dec-(2E)-ylidene]-amide

This compound is prepared analogously to Example 51 by replacing 4-(benzyloxy)benzoic acid with 2,3-Dimethyl-1-[2-(tetrahydro-pyran-2-yloxy)-ethyl]-1H-indole-5-carboxylic acid (Intermediate AU); [M-4-1]⁺540.

### Example 89

### 4-(4-{2-[(E)-3,5-Diamino-6-chloro-pyrazine-2-carbonylimino]-1,3,8-triaza-spiro[4.5]decane-8-carbonyl}-indol-1-yl)-butyric acid methyl ester

This compound is prepared analogously to Example 51 by replacing 4-(benzyloxy)benzoic acid with 1-(4,4,4-Trimethoxy-butyl)-1H-indole-4-carboxylic acid (Intermediate AW); [M+H]⁺568

### Example 90

### 3,5-Diamino-6-chloro-pyrazine-2-carboxylic acid [8-{1-[2-(2-methoxy-ethoxymethoxy)-ethyl]-1H-indole-4-carbonyl}-1,3,8-triaza-spiro[4.5]dec-(2E)-ylidene]-amide

This compound is prepared analogously to Example 51 by replacing 4-(benzyloxy)benzoic acid with 1-[2-(2-Methoxy-ethoxymethoxy)-ethyl]-1H-indole-4-carboxylic acid (Intermediate AW); [M+H]⁺600

### Example 91

### 3,5-Diamino-6-chloro-pyrazine-2-carboxylic acid [8-(1-diethylcarbamoylmethyl-1H-indole-4-carbonyl)-1,3,8-triaza-spiro[4.5]dec-(2E)-ylidene]-amide

This compound is prepared analogously to Example 51 by replacing 4-(benzyloxy)benzoic acid with 1-Diethylcarbamoylmethyl-1H-indole-4-carboxylic acid (Intermediate AX); [M+H]⁺581

### Example 92

### 3,5-Diamino-6-chloro-pyrazine-2-carboxylic acid [8-[1-(2-hydroxy-ethyl)-1H-indole-4-carbonyl]-1,3,8-triaza-spiro[4.5]dec-(2E)-ylidene]-amide

p-Toluenesulfonic acid monohydrate (1.6 mg, 0.0084 mmol) is added to a stirred solution of 3,5-diamino-6-chloro-pyrazine-2-carboxylic acid [8-{1-[2-(tetrahydro-pyran-2-yloxy)-ethyl]-1H-indole-4-carbonyl}-1,3,8-triaza-spiro[4.5]dec-(2E)-ylidene]-amide (Ex. 84) (50 mg, 0.084 mmol) in MeOH (3 ml) and the resulting solution is stirred at room temperature for 3 hrs, then heated at 50° C. for 16 hours. The solvent is removed in vacuo and the residue is dissolved in MeOH (3 ml) and loaded onto a 1 g PEAX cartridge which is eluted with MeOH (20 ml). The filtrate is concentrated in vacuo to afford the title compound; [M+H]⁺512/514

### Example 93

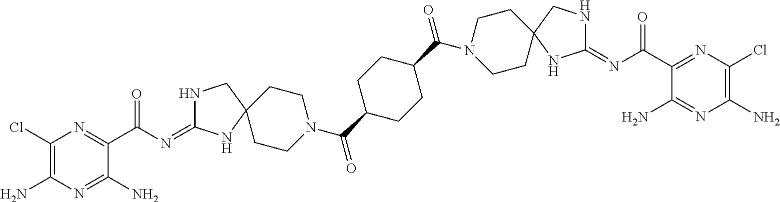 [SMILES: Nc1nc(N)c(C(=O)/N=C2\NCC3(CCN(C(=O)[C@H]4CC[C@@H](C(=O)N5CCC6(CC5)CN/C(=N\C(=O)c5nc(Cl)c(N)nc5N)N6)CC4)CC3)N2)nc1Cl]

A mixture of 3,5-diamino-6-chloro-pyrazine-2-carboxylic acid [1,3,8-triazaspiro[4.5]dec-(2E)-ylidene]-amide dihydrochloride (Ex. 38) (300 mg, 0.83 mmol), cis-1,4-cyclohexanedicarboxylic acid (72 mg, 0.42 mmol), N-methyl morpholine (0.30 ml, 2.73 mmol) and HATU (315 mg, 0.83 mmol) in anhydrous DMF is stirred at room temperature for 16 hours. The reaction mixture is concentrated in vacuo and is subjected to column chromatography (basic alumina, 0-3% MeOH in DCM) to obtain off-white solid. The product is dissolved in DCM and re-precipitated by addition of diethyl ether. The supernatant solvent mixture is decanted and the product is washed again with diethyl ether and dried under vacuum to afford the compound shown as off-white solid; [M+H]⁺785.

### Example 94

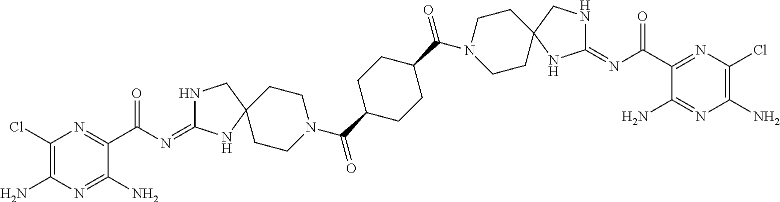 [SMILES: Nc1nc(N)c(C(=O)/N=C2\NCC3(CCN(C(=O)[C@H]4CC[C@@H](C(=O)N5CCC6(CC5)CN/C(=N\C(=O)c5nc(Cl)c(N)nc5N)N6)CC4)CC3)N2)nc1Cl]

This compound is prepared analogously to Example 93 by replacing cis-1,4-cyclohexanedicarboxylic acid with trans-1,4-cyclohexanedicarboxylic acid; [M+2H]²⁺393.

### Example 95

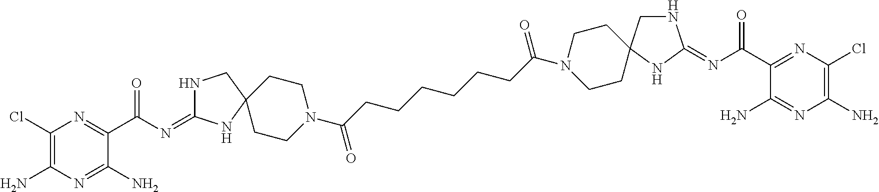 [SMILES: Nc1nc(N)c(C(=O)/N=C2\NCC3(CCN(C(=O)CCCCCCC(=O)N4CCC5(CC4)CN/C(=N\C(=O)c4nc(Cl)c(N)nc4N)N5)CC3)N2)nc1Cl]

This compound is prepared analogously to Example 93 by replacing cis-1,4-cyclohexanedicarboxylic acid with suberic acid; [M+H]⁺787.

### Example 96

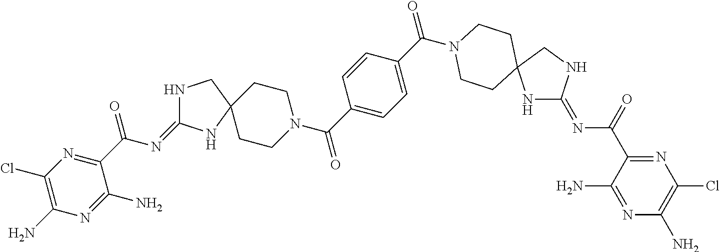 [SMILES: Nc1nc(N)c(C(=O)/N=C2\NCC3(CCN(C(=O)c4ccc(C(=O)N5CCC6(CC5)CN/C(=N\C(=O)c5nc(Cl)c(N)nc5N)N6)cc4)CC3)N2)nc1Cl]

This compound is prepared analogously to Example 93 by replacing cis-1,4-cyclohexanedicarboxylic acid with terephthalic acid; [M+H]⁺779.

### Example 97

### 3,5-Diamino-6-chloro-pyrazine-2-carboxylic acid [8-[2-(4-benzyloxy-phenyl)-acetyl]-1,3,8-triaza-spiro[4.5]dec-(2E)-ylidene]-amide

A mixture of 3,5-diamino-6-chloro-pyrazine-2-carboxylic acid [1,3,8-triaza-spiro[4.5]dec-(2E)-ylidene]-amide dihydrochloride (Ex. 38) (300 mg, 0.83 mmol), 4-benzyloxyphenylacetic acid (200 mg, 0.83 mmol), N-methyl morpholine (0.40 ml, 3.64 mmol) and HATU (315 mg, 0.83 mmol) in anhydrous DMF (20 ml) is stirred at room temperature for 16 hours. The reaction mixture is concentrated in vacuo and subjected to column chromatography (basic alumina, 0-3% MeOH in DCM) to obtain pale yellow solid. The product is dissolved in DCM and MeOH and re-precipitated by adding diethyl ether. The supernatant solvent mixture is decanted and the product is washed again with diethyl ether and dried under vacuum to afford the title compound as a pale yellow solid; [M+H]⁺549.

### Example 98

### 3,5-Diamino-6-chloro-pyrazine-2-carboxylic acid [8-[3-(4-benzyloxy-phenyl)-propionyl]-1,3,8-triaza-spiro[4.5]dec-(2E)-ylidene]-amide

This compound is prepared analogously to Example 97 by replacing 4-benzyloxyphenylacetic acid with 3-(4-benzyloxyphenyl)propionic acid; [M+H]⁺563.

### Example 99

### 3,5-Diamino-6-chloro-pyrazine-2-carboxylic acid [8-(1H-indole-4-carbonyl)-1,3,8-triaza-spiro[4.5]dec-(2E)-ylidene]-amide

This compound is prepared analogously to Example 97 by replacing 4-benzyloxyphenylacetic acid with indole-4-carboxylic acid; [M+H]⁺468.

### Example 100

### 3,5-Diamino-6-chloro-pyrazine-2-carboxylic acid [8-(1H-indole-5-carbonyl)-1,3,8-triaza-spiro[4.5]dec-(2E)-ylidene]-amide

This compound is prepared analogously to Example 97 by replacing 4-benzyloxyphenylacetic acid with indole-5-carboxylic acid; [M+H]⁺468.

### Example 101

### 3,5-Diamino-6-chloro-pyrazine-2-carboxylic acid [8-phenylacetyl-1,3,8-triaza-spiro[4.5]dec-(2E)-ylidene]-amide

This compound is prepared analogously to Example 97 by replacing 4-benzyloxyphenylacetic acid with phenylacetic acid; [M+H]⁺443.

### Example 102

### 3,5-Diamino-6-chloro-pyrazine-2-carboxylic acid [8-{4-[6-((S)-2,3-dihydroxy-propoxy)-naphthalen-2-ylmethoxy]-benzoyl}-1,3,8-triaza-spiro[4,5]dec-(2E)-ylidene]-amide

### Step 1

3,5-Diamino-6-chloro-pyrazine-2-carboxylic acid [8-{4-[6-((R)-2,2-dimethyl-[1,3]dioxolan-4-ylmethoxy)-naphthalen-2-ylmethoxy]-benzoyl}-1,3,8-triaza-spiro[4.5]dec-(2E)-ylidene]-amide is prepared analogously to Example 97 by replacing 4-benzyloxyphenylacetic acid with 4-[6-((R)-2,2-dimethyl-[1,3]dioxolan-4-ylmethoxy)-naphthalen-2-ylmethoxy]-benzoic acid (Intermediate AY); [M+H]⁺715.

### Step 2:

To a solution of 3,5-diamino-6-chloro-pyrazine-2-carboxylic acid [8-{4-[6-((R)-2,2-dimethyl-[1,3]dioxolan-4-ylmethoxy)-naphthalen-2-ylmethoxy]-benzoyl}-1,3,8-triaza-spiro[4.5]dec-(2E)-ylidene]-amide (0.16 g, 0.22 mmol) in MeOH (10 ml) is added SCX-2 resin (−2 g), the resultant slurry is stirred for 0.5 hours and then the solvent is removed in vacuo. The slurry is loaded onto a column of SCX-2 resin (˜3 g) and eluted with MeOH and then with 2 M NH₃ in MeOH. The methanolic ammonia fractions are concentrated in vacuo and the residue is triturated with diethyl ether to obtain 3,5-Diamino-6-chloro-pyrazine-2-carboxylic acid [8-{4-[6-((S)-2,3-dihydroxy-propoxy)-naphthalen-2-ylmethoxy]-benzoyl}-1,3,8-triaza-spiro[4.5]dec-(2E)-ylidene]-amide as yellow solid; [M+H]⁺675.

### Example 103

### 3,5-Diamino-6-chloro-pyrazine-2-carboxylic acid [8-(4-chloro-benzoyl)-1,3,8-triaza-spiro[4.5]dec-(2E)-ylidene]-amide

This compound is prepared analogously to Example 97 by replacing 4-benzyloxyphenylacetic acid with p-chlorobenzoic acid; [M+H]⁺463.

### Example 104

### 3,5-Diamino-6-chloro-pyrazine-2-carboxylic acid [8-(4-{3-[4-((5)-2,3-dihydroxy-propoxy)-phenyl]-propoxy}-benzoyl)-1,3,8-triaza-spiro[4,5]dec-(2E)-ylidene]-amide

This compound is prepared analogously to Example 102 by replacing 4-[6-((R)-2,2-dimethyl-[1,3]dioxolan-4-ylmethoxy)-naphthalen-2-ylmethoxy]-benzoic acid, (Intermediate AY) with 4-{3-[4-((R)-2,2-dimethyl-[1,3]dioxolan-4-ylmethoxy)-phenyl]-propoxy}-benzoic acid (Intermediate AZ); [M+H]⁺653.

### Example 105

### 3,5-Diamino-6-chloro-pyrazine-2-carboxylic acid [8-[(E)-(3-phenyl-acryloyl)]-1,3,8-triaza-spiro[4.5]dec-(2E)-ylidene]-amide

This compound is prepared analogously to Example 97 by replacing 4-benzyloxyphenylacetic acid with trans-cinnamic acid; [M+H]⁺455.

### Example 106

### 3,5-Diamino-6-chloro-pyrazine-2-carboxylic acid [8-benzoyl-1,3,8-triaza-spiro[4.5]dec-(2E)-ylidene]-amide

This compound is prepared analogously to Example 97 by replacing 4-benzyloxyphenylacetic acid with benzoic acid; [M+H]⁺429.

### Example 107

### 3,5-Diamino-6-chloro-pyrazine-2-carboxylic acid [8-(benzofuran-5-carbonyl)-1,3,8-triaza-spiro[4.5]dec-(2E)-ylidene]-amide

This compound is prepared analogously to Example 97 by replacing 4-benzyloxyphenylacetic acid with benzofuran-5-carboxylic acid; [M+H]⁺469.

### Example 108

### 3,5-Diamino-6-chloro-pyrazine-2-carboxylic acid [8-hexanoyl-1,3,8-triaza-spiro[4.5]dec-(2E)-ylidene]-amide

This compound is prepared analogously to Example 97 by replacing 4-benzyloxyphenylacetic acid with hexanoic acid; [M+H]⁺423.

### Example 109

### 3,5-Diamino-6-chloro-pyrazine-2-carboxylic acid [8-(3-phenyl-propynoyl)-1,3,8-triaza-spiro[4.5]dec-(2E)-ylidene]-amide

This compound is prepared analogously to Example 97 by replacing 4-benzyloxyphenylacetic acid with phenylpropiolic acid; [M+H]⁺453.

### Example 110

### 3,5-Diamino-6-chloro-pyrazine-2-carboxylic acid [8-(1H-imidazole-2-carbonyl)-1,3,8-triaza-spiro[4.5]dec-(2E)-ylidene]-amide

This compound is prepared analogously to Example 97 by replacing 4-benzyloxyphenylacetic acid with 2-imidazolecarboxylic acid; [M+H]⁺419.

### Example 111

### 3,5-Diamino-6-chloro-pyrazine-2-carboxylic acid [8-isobutyryl-1,3,8-triaza-spiro[4.5]dec-(2E)-ylidene]-amide

This compound is prepared analogously to Example 97 by replacing 4-benzyloxyphenylacetic acid with isobuteric acid; [M+H]⁺395.

### Example 112

### 3,5-Diamino-6-chloro-pyrazine-2-carboxylic acid [8-(4-cyano-benzoyl)-1,3,8-triaza-spiro[4.5]dec-(2E)-ylidene]-amide

This compound is prepared analogously to Example 97 by replacing 4-benzyloxyphenylacetic acid with p-cyanobenzoic acid; [M+H]⁺454.

### Example 113

### 3,5-Diamino-6-chloro-pyrazine-2-carboxylic acid [8-(pyridine-3-carbonyl)-1,3,8-triaza-spiro[4.5]dec-(2E)-ylidene]-amide

This compound is prepared analogously to Example 97 by replacing 4-benzyloxyphenylacetic acid with nicotinic acid; [M+H]⁺430.

### Example 114

### 4-{2-[(E)-3,5-Diamino-6-chloro-pyrazine-2-carbonylimino]-1,3,8-triaza-spiro[4.5]decane-8-carbonyl}-benzoic acid methyl ester

This compound is prepared analogously to Example 97 by replacing 4-benzyloxyphenylacetic acid with monomethyl terephthalate; [M+H]⁺487.

### Example 115

### 3,5-Diamino-6-chloro-pyrazine-2-carboxylic acid [8-(pyrimidine-5-carbonyl)-1,3,8-triaza-spiro[4.5]dec-(2E)-ylidene]-amide

This compound is prepared analogously to Example 97 by replacing 4-benzyloxyphenylacetic acid with pyrimidine-5-carboxylic acid; [M+H]⁺431.

### Example 116

### 3,5-Diamino-6-chloro-pyrazine-2-carboxylic acid [8-(4-hydroxy-benzoyl)-1,3,8-triaza-spiro[4.5]dec-(2E)-ylidene]-amide

This compound is prepared analogously to Example 97 by replacing 4-benzyloxyphenylacetic acid with 4-hydroxybenzoic acid; [M+H]⁺445.

### Example 117

### 3,5-Diamino-6-chloro-pyrazine-2-carboxylic acid [8-cyclohexanecarbonyl-1,3,8-triaza-spiro[4.5]dec-(2E)-ylidene]-amide

This compound is prepared analogously to Example 97 by replacing 4-benzyloxyphenylacetic acid with cyclohexanecarboxylic acid; [M+H]⁺435.

### Example 118

### 3,5-Diamino-6-chloro-pyrazine-2-carboxylic acid [8-(oxazole-4-carbonyl)-1,3,8-triaza-spiro[4.5]dec-(2E)-ylidene]-amide

This compound is prepared analogously to Example 97 by replacing 4-benzyloxyphenylacetic acid with oxazole-4-carboxylic acid; [M+H]⁺420.

### Example 119

### 3,5-Diamino-6-chloro-pyrazine-2-carboxylic acid [8-(pyridine-2-carbonyl)-1,3,8-triaza-spiro[4.5]dec-(2E)-ylidene]-amide

This compound is prepared analogously to Example 97 by replacing 4-benzyloxyphenylacetic acid with 2-picolinic acid; [M+H]⁺430.

### Example 120

### 3,5-Diamino-6-chloro-pyrazine-2-carboxylic acid [8-(pyridine-4-carbonyl)-1,3,8-triaza-spiro[4.5]dec-(2E)-ylidene]-amide

This compound is prepared analogously to Example 97 by replacing 4-benzyloxyphenylacetic acid with isonicotinic acid; [M+H]⁺430.

### Example 121

### 3,5-Diamino-6-chloro-pyrazine-2-carboxylic acid [8-(piperidine-4-carbonyl)-1,3,8-triaza-spiro[4.5]dec-(2E)-ylidene]-amide hydrochloride

4 M HCl in dioxane (5 ml) is added to a solution of 4-{2-[(E)-3,5-diamino-6-chloro-pyrazine-2-carbonylimino]-1,3,8-triaza-spiro[4.5]decane-8-carbonyl}-piperidine-1-carboxylic acid tert-butyl ester (Intermediate BA) (0.14 g, 0.26 mmol) in dioxane (10 ml) and the reaction mixture is stirred at room temperature for 3 hours. The reaction mixture is concentrated in vacuo and the yellow solid obtained is triturated with DCM. The DCM layer is decanted and the compound is washed with MeOH and dried under vacuum to afford the title compound as yellow solid; [M+H]⁺436.

### Example 122

### 3,5-Diamino-6-chloro-pyrazine-2-carboxylic acid [8-(1H-imidazole-4-carbonyl)-1,3,8-triaza-spiro[4.5]dec-(2E)-ylidene]-amide

This compound is prepared analogously to Example 97 by replacing 4-benzyloxyphenylacetic acid with 4-imidazolecarboxylic acid; [M+H]⁺419.

### Example 123

### 3,5-Diamino-6-chloro-pyrazine-2-carboxylic acid [8-(tetrahydro-pyran-4-carbonyl)-1,3,8-triaza-spiro[4.5]dec-(2E)-ylidene]-amide

This compound is prepared analogously to Example 97 by replacing 4-benzyloxyphenylacetic acid with tetrahydropyran-4-carboxylic acid; [M+H]⁺437.

### Example 124

### 3,5-Diamino-6-chloro-pyrazine-2-carboxylic acid [8-(pyrimidine-4-carbonyl)-1,3,8-triaza-spiro[4.5]dec-(2E)-ylidene]-amide

This compound is prepared analogously to Example 97 by replacing 4-benzyloxyphenylacetic acid with pyrimidine-4-carboxylic acid; [M+H]⁺431.

### Example 125

### 3,5-Diamino-6-chloro-pyrazine-2-carboxylic acid [8-(oxazole-5-carbonyl)-1,3,8-triaza-spiro[4.5]dec-(2E)-ylidene]-amide

This compound is prepared analogously to Example 97 by replacing 4-benzyloxyphenylacetic acid with oxazole-5-carboxylic acid; [M+H]⁺420.

### Example 126

### 3,5-Diamino-6-chloro-pyrazine-2-carboxylic acid [8-[3-(4-isobutoxy-piperidine-1-sulfonyl)-benzoyl]-1,3,8-triaza-spiro[4.5]dec-(2E)-ylidene]-amide

### Step 1

A solution of N,N-Diisopropylethylamine (0.0078 ml, 0.045 mmol) in THF (1 ml) is added to 4-Isobutoxy-piperidine (0.008 g, 0.05 mmol) followed by a solution of 3-(Chlorosulfonyl)benzoic acid (9.93 mg, 0.045 mmol) and shaken at room temperature for 48 hours. The solution is evaporated under vacuum to afford 3-(4-Isobutoxy-piperidine-1-sulfonyl)-benzoic acid which is used without purification; [M+H]⁺342.00.

### Step 2

3-(4-Isobutoxy-piperidine-1-sulfonyl)-benzoic acid (0.03 mmol, 10.2 mg) is treated with a solution of HATU (11.4 mg, 0.03 mmol) in DMF (1 ml) followed by a solution of 3,5-Diamino-6-chloro-pyrazine-2-carboxylic acid [1,3,8-triaza-spiro[4.5]dec-(2E)-ylidene]-amide dihydrochloride (Ex. 38) (11.9 mg, 0.03 mmol) and N-methyl morpholine (0.010 ml, 0.03 mmol) in DMF (1 ml) and shaken at room temperature overnight. The solution is evaporated under vacuum, redissolved in DMSO (0.5 ml) and purified by mass-directed preparative HPLC. The purified fractions are evaporated under vacuum to afford the title compound; [M+H]⁺648.4.

### Examples 127-145

These compounds, namely 3,5-Diamino-6-chloro-pyrazine-2-carboxylic acid [8-{3-[2-(1H-indol-3-yl)-ethylsulfamoyl]-benzoybenzoyl}-1,3,8-triaza-spiro[4.5]dec-(2E)-ylidene]-amide (Example 127); 1-(3-{2-[(E)-3,5-Diamino-6-chloro-pyrazine-2-carbonylimino]-1,3,8-triaza-spiro[4.5]decane-8-carbonyl}-benzenesulfonyl)-piperidine-3-carboxylic acid ethyl ester (Example. 128);
3,5-Diamino-6-chloro-pyrazine-2-carboxylic acid [8-(3-cyclopentylsulfamoyl-benzoyl)-1,3,8-triaza-spiro[4.5]dec-(2E)-ylidene]-amide (Example 127);
3,5-Diamino-6-chloro-pyrazine-2-carboxylic acid [8-(3-(1-acetyl-piperidin-4-ylsulfamoyl)-benzoyl]-1,3,8-triaza-spiro[4.5]dec-(2E)-ylidene]-amide (Example 130);
3,5-Diamino-6-chloro-pyrazine-2-carboxylic acid [8-{3-[(tetrahydro-furan-2-ylmethyl)-sulfamoyl]-benzoybenzoyl}-1,3,8-triaza-spiro[4.5]dec-(2E)-ylidene]-amide (Example 131);
3,5-Diamino-6-chloro-pyrazine-2-carboxylic acid [8-{3-[(pyridin-3-ylmethyl)-sulfamoyl]-benzoybenzoyl}-1,3,8-triaza-spiro[4.5]dec-(2E)-ylidene]-amide (Example 132);
3,5-Diamino-6-chloro-pyrazine-2-carboxylic acid [8-{3-[(2,2-dimethoxy-ethyl)-methyl-sulfamoyl]-benzoybenzoyl}-1,3,8-triaza-spiro[4.5]dec-(2E)-ylidene]-amide (Example 133);
3,5-Diamino-6-chloro-pyrazine-2-carboxylic acid [8-[3-(2,4-difluoro-benzylsulfamoyl)-benzoyl]-1,3,8-triaza-spiro[4.5]dec-(2E)-ylidene]-amide (Example 134);
3,5-Diamino-6-chloro-pyrazine-2-carboxylic acid [8-[3-(1-pyridin-4-yl-ethylsulfamoyl)-benzoyl]-1,3,8-triaza-spiro[4.5]dec-(2E)-ylidene]-amide (Example 135);
3,5-Diamino-6-chloro-pyrazine-2-carboxylic acid [8-[3-(2-phenyl-morpholine-4-sulfonyl)-benzoyl]-1,3,8-triaza-spiro[4.5]dec-(2E)-ylidene]-amide (Example 136);
3,5-Diamino-6-chloro-pyrazine-2-carboxylic acid [8-[3-(3-difluoromethoxy-benzylsulfamoyl)-benzoyl]-1,3,8-triaza-spiro[4.5]dec-(2E)-ylidene]-amide (Example 137);
3,5-Diamino-6-chloro-pyrazine-2-carboxylic acid [8-[3-(4-pyrrolidin-1-yl-piperidine-1-sulfonyl)-benzoyl]-1,3,8-triaza-spiro[4.5]dec-(2E)-ylidene]-amide (Example 138);
3,5-Diamino-6-chloro-pyrazine-2-carboxylic acid [8-{3-[(5-methyl-pyrazin-2-ylmethyl)-sulfamoyl]-benzoybenzoyl}-1,3,8-triaza-spiro[4.5]dec-(2E)-ylidene]-amide (Example 139);
3,5-Diamino-6-chloro-pyrazine-2-carboxylic acid [8-[3-(dimethylcarbamoylmethyl-sulfamoyl)-benzoyl]-1,3,8-triaza-spiro[4.5]dec-(2E)-ylidene]-amide (Example 140);
3,5-Diamino-6-chloro-pyrazine-2-carboxylic acid [8-[3-(3-benzenesulfonyl-pyrrolidine-1-sulfonyl)-benzoyl]-1,3,8-triaza-spiro[4.5]dec-(2E)-ylidene]-amide (Example 141);
3,5-Diamino-6-chloro-pyrazine-2-carboxylic acid [8-{3-[([1,3]dioxolan-2-ylmethyl)-sulfamoyl]-benzoybenzoyl}-1,3,8-triaza-spiro[4.5]dec-(2E)-ylidene]-amide (Example 142);
3,5-Diamino-6-chloro-pyrazine-2-carboxylic acid [8-{3-[2-(pyridin-3-yloxy)-propylsulfamoyl]-benzoybenzoyl}-1,3,8-triaza-spiro[4.5]dec-(2E)-ylidene]-amide (Example 143);
3,5-Diamino-6-chloro-pyrazine-2-carboxylic acid [8-{3-[4-(5-trifluoromethyl-pyridin-2-yl)-[1,4]diazepane-1-sulfonylFbenzoybenzoyl}-1,3,8-triaza-spiro[4.5]dec-(2E)-ylidene]-amide (Example 144);
3,5-Diamino-6-chloro-pyrazine-2-carboxylic acid [8-[3-(1,1-dioxo-tetrahydro-1lambda*6*-thiophen-3-ylsulfamoyl)-benzoyl]-1,3,8-triaza-spiro[4.5]dec-(2E)-ylidene]-amide (Example 145); are made analogously to Examples 126 replacing 4-isobutoxy-piperidine in step 1 with the appropriate amines which are all commercially available. The compounds are recovered from the reaction mixture and purified using conventional techniques.

### Example 146

### 3,5-Diamino-6-chloro-pyrazine-2-carboxylic acid [8-{3-[3-(4-chlorophenyl)-[1,2,4]oxadiazol-5-yl]-propionyl}-1,3,8-triaza-spiro[4.5]dec-(2E)-ylidene-amide trifluoroacetate

N-methyl morpholine (33 μl, 0.3 mmol) is added to 3-(3-p-Tolyl-[1,2,4]oxadiazol-5-yl)-propionic acid (0.1 mmol), followed by HATU (41.8 mg, 0.11 mmol) dissolved in peptide grade DMF (250 μl) and 3,5-Diamino-6-chloro-pyrazine-2-carboxylic acid [1,3,8-tiaza-spiro[4.5]dec-(2E)-ylidene]-amide dihydrochloride (Ex. 38) (40 mg, 0.1 mmol) dissolved in peptide grade DMF (250 μl). The reaction is sealed and shaken overnight at room temperature. Purification is by mass-directed preparative HPLC to give the title compound; [M+H]⁺559.3.

### Example 147

### 3,5-Diamino-6-chloro-pyrazine-2-carboxylic acid [8-[1-(toluene-4-sulfonyl)-1H-pyrrole-3-carbonyl]-1,3,8-triaza-spiro[4.5]dec-(2E)-ylidene]-amide

A solution of 1-(Toluene-4-sulfonyl)-1H-pyrrole-3-carboxylic acid (0.023 g, 0.085 mmol) in NMP (850 μl) is added to PS-carbodiimide (190 mg of 1.3 mmol/g loading, 0.24 mmol), followed by a solution of 3,5-Diamino-6-chloro-pyrazine-2-carboxylic acid [1,3,8-triaza-spiro[4.5]dec-(2E)-ylidene]-amide dihydrochloride (Ex. 38) (0.08 mmol) and N-methyl morpholine (8 μl, 0.08 mmol) in NMP (1 ml), and the resulting reaction mixture is shaken at room temperature. The reaction mixture is filtered and the resin is washed with NMP (1 ml). The collected filtrate is concentrated in vacuo and the residues are purified by mass-directed preparative HPLC. The purified fractions are evaporated under vacuum to afford the title compound; [M+H]⁺572.08.

### Example 148

### 3,5-Diamino-6-chloro-pyrazine-2-carboxylic acid [8-[1-(3,4-difluoro-benzyl)-6-oxo-1,6-dihydro-pyridine-3-carbonyl]-1,3,8-triaza-spiro[4.5]dec-(2E)-ylidene]-amide

A solution of 1-(3,4-Difluoro-benzyl)-6-oxo-1,6-dihydro-pyridine-3-carboxylic acid (0.15 mmol) in NMP (0.5 ml) is added to a solution of 3,5-Diamino-6-chloro-pyrazine-2-carboxylic acid [1,3,8-triaza-spiro[4.5]dec-(2E)-ylidene]-amide dihydrochloride (Ex. 38) (0.049 g, 0.15 mmol) and N-methyl morpholine (0.033 ml, 0.30 mmol) in NMP (1 ml), followed by a solution of HATU (0.11 g, 0.3 mmol) in NMP (0.5 ml). The reaction mixture is shaken at room temperature overnight. The reaction mixture is purified by mass-directed preparative HPLC. Fractions containing pure product are eluted through SCX-2 cartridges (Biotage 1 g/6 ml cartridge), and the cartridge is washed with MeOH (4 ml), followed by 3M NH₃ in MeOH solution (4 ml) to afford the title compound; [M+H]⁺572.0.

### Examples 149-213

Exemplary compounds,
namely 3,5-Diamino-6-chloro-pyrazine-2-carboxylic acid [8-[4-(3-phenyl-isoxazol-5-yl)-butyryl]-1,3,8-triaza-spiro[4.5]dec-(2E)-ylidene-amide (Example 149);
3,5-Diamino-6-chloro-pyrazine-2-carboxylic acid [8-[4-(5-fluoro-2,3-dihydro-indol-1yl)-4-oxo-butyryl]-1,3,8-triaza-spiro[4.5]dec-(2E)-ylidene-amide (Example 150);
3,5-Diamino-6-chloro-pyrazine-2-carboxylic acid [8-{4-[3-(4-methoxy-phenyl)-[1,2,4]oxadiazol-5yl]-butyryl}-1,3,8-triaza-spiro[4.5]dec-(2E)-ylidene-amide (Example 151);
3,5-Diamino-6-chloro-pyrazine-2-carboxylic acid [8-(4-1H-indazol-3-yl-butyryl)-1,3,8-triaza-spiro[4.5]dec-(2E)-ylidene-amide (Example 152);
3,5-Diamino-6-chloro-pyrazine-2-carboxylic acid [8-[4-(5-methanesulfonyl-2,3-dihydro-indol-1yl)-4-oxo-butyryl]-1,3,8-triaza-spiro[4.5]dec-(2E)-ylidene-amide (Example 153);
3,5-Diamino-6-chloro-pyrazine-2-carboxylic acid [8-(4-benzothiazol-2-yl-butyryl)-1,3,8-triaza-spiro[4.5]dec-(2E)-ylidene-amide (Example 154);
3,5-Diamino-6-chloro-pyrazine-2-carboxylic acid [8-[4-(5-dimethylsulfamoyl-2,3-dihydro-indol-1yl)-4-oxo-butyryl]-1,3,8-triaza-spiro[4.5]dec-(2E)-ylidene-amide (Example 155);
3,5-Diamino-6-chloro-pyrazine-2-carboxylic acid [8-[4-(2-oxo-2,3-dihydro-1H-indol-3yl)-butyryl]-1,3,8-triaza-spiro[4.5]dec-(2E)-ylidene-amide (Example 156);
3,5-Diamino-6-chloro-pyrazine-2-carboxylic acid [8-[4-(6-dimethylamino-9H-purin-8yl)-butryrl]-1,3,8-triaza-spiro[4.5]dec-(2E)-ylidene-amide (Example 157);
3,5-Diamino-6-chloro-pyrazine-2-carboxylic acid [8-[4-(2-oxo-3-pyridin-3yl-2,3-dihydro-benzoimidazol-1-yl)-butyryl]-1,3,8-triaza-spiro[4.5]dec-(2E)-ylidene-amide (Example 158);
3,5-Diamino-6-chloro-pyrazine-2-carboxylic acid [8-[4-(2-oxo-3-pyridine-3ylmethyl-2,3-dihydro-indol-1-yl)-butryrl]-1,3,8-triaza-spiro[4.5]dec-(2E)-ylidene-amide (Example 159);
3,5-Diamino-6-chloro-pyrazine-2-carboxylic acid [8-[4-(9-oxo-3,3a,4,9,10,10a-hexahydro-1H-2-aza-benzol[F]azulen-2yl)-butyryl]-1,3,8-triaza-spiro[4.5]dec-(2E)-ylidene-amide (Example 160);
3,5-Diamino-6-chloro-pyrazine-2-carboxylic acid [8-[4-(6-amino-9H-purine-8yl)-butyryl]-1,3,8-triaza-spiro[4.5]dec-(2E)-ylidene-amide (Example 161);
3,5-Diamino-6-chloro-pyrazine-2-carboxylic acid [8-(4-oxo-4-pyrrolidin-1-yl-butyryl)-1,3,8-triaza-spiro[4.5]dec-(2E)-ylidene-amide (Example 162);
3,5-Diamino-6-chloro-pyrazine-2-carboxylic acid [8-(4-[1,2,4]triazol-1-yl-butyryl)-1,3,8-triaza-spiro[4.5]dec-(2E)-ylidene-amide (Example 163);
3,5-Diamino-6-chloro-pyrazine-2-carboxylic acid [8-(5-dibenzylsulfamoyl-1-methyl-1H-pyrrole-2-carbonyl)-1,3,8-triaza-spiro[4.5]dec-(2E)-ylidene]-amide (Example 164);
3,5-Diamino-6-chloro-pyrazine-2-carboxylic acid [8-{4-[3-(4-chloro-phenyl)-[1,2,4]oxadiazol-5-yl]-butyrybenzoyl}-1,3,8-triaza-spiro[4.5]dec-(2E)-ylidene]-amide (Example 165);
3,5-Diamino-6-chloro-pyrazine-2-carboxylic acid [8-{4-[(naphthalene-1-sulfonylamino)-methyl]-benzoybenzoyl}-1,3,8-triaza-spiro[4.5]dec-(2E)-ylidene]-amide (Example 166);
3,5-Diamino-6-chloro-pyrazine-2-carboxylic acid [8-{2[3-(4-chlorophenyl)-[1,2,4]oxadiazol-5-ylFacetybenzoyl}-1,3,8-triaza-spiro[4.5]dec-(2E)-ylidene-amide (Example 167);
3,5-Diamino-6-chloro-pyrazine-2-carboxylic acid [8-[3-(3-methoxy-propoxy)-benzoyl]-1,3,8-triaza-spiro[4.5]dec-(2E)-ylidene]-amide (Example 168);
3,5-Diamino-6-chloro-pyrazine-2-carboxylic acid [8-(2-benzotriazol-2-yl-acetyl)-1,3,8-triaza-spiro[4.5]dec-(2E)-ylidene]-amide (Example 169)
3,5-Diamino-6-chloro-pyrazine-2-carboxylic acid [8-(2-benzotriazol-2-yl-acetyl)-1,3,8-triaza-spiro[4.5]dec-(2E)-ylidene]-amide (Example 170);
3,5-Diamino-6-chloro-pyrazine-2-carboxylic acid [8-[4-(2-isopropoxy-ethylamino)-benzoyl]-1,3,8-triaza-spiro[4.5]dec-(2E)-ylidene]-amide (Example 171);
3,5-Diamino-6-chloro-pyrazine-2-carboxylic acid [8-[6-oxo-1-(3-trifluoromethyl-benzyl)-1,6-dihydro-pyridine-3-carbonyl]-1,3,8-triaza-spiro[4.5]dec-(2E)-ylidene]-amide (Example 172);
3,5-Diamino-6-chloro-pyrazine-2-carboxylic acid [8-[6-(4-methyl-piperazin-1-yl)-pyridine-3-carbonyl]-1,3,8-triaza-spiro[4.5]dec-(2E)-ylidene]-amide (Example 173);
3,5-Diamino-6-chloro-pyrazine-2-carboxylic acid [8-[3-(4-fluoro-phenyl)-5-methyl-isoxazole-4-carbonyl]-1,3,8-triaza-spiro[4.5]dec-(2E)-ylidene]-amide (Example 174);
3,5-Diamino-6-chloro-pyrazine-2-carboxylic acid [8-{3-[3-(4-methoxy-phenyl)-[1,2,4]oxadiazol-5-yl]-propionyll-1,3,8-triaza-spiro[4.5]dec-(2E)-ylidene]-amide (Example 175);
3,5-Diamino-6-chloro-pyrazine-2-carboxylic acid [8-[2-(4-trifluoromethoxy-phenoxy)-acetyl]-1,3,8-triaza-spiro[4.5]dec-(2E)-ylidene]-amide (Example 176);
3,5-Diamino-6-chloro-pyrazine-2-carboxylic acid [8-{2-[4-(2-oxo-imidazolidin-1-yl)-phenyl]-acetybenzoyl}-1,3,8-triaza-spiro[4.5]dec-(2E)-ylidene]-amide (Example 177);
3,5-Diamino-6-chloro-pyrazine-2-carboxylic acid [8-[3-(3-phenyl-isoxazol-5-yl)-propionyl]-1,3,8-triaza-spiro[4.5]dec-(2E)-ylidene]-amide (Example 178);
3,5-Diamino-6-chloro-pyrazine-2-carboxylic acid [8-[2-(4-methanesulfonyl-phenyl)-acetyl]-1,3,8-triaza-spiro[4.5]dec-(2E)-ylidene]-amide (Example 179);
3,5-Diamino-6-chloro-pyrazine-2-carboxylic acid [8-[2-(4-chloro-phenyl)-thiazole-4-carbonyl]-1,3,8-triaza-spiro[4.5]dec-(2E)-ylidene]-amide (Example 180);
3,5-Diamino-6-chloro-pyrazine-2-carboxylic acid [8-[2-(5-methyl-3-trifluoromethyl-pyrazol-1-yl)-acetyl]-1,3,8-triaza-spiro[4.5]dec-(2E)-ylidene]-amide (Example 181);
3,5-Diamino-6-chloro-pyrazine-2-carboxylic acid [8-[5-(pyridin-3-yloxy)-furan-2-carbonyl]-1,3,8-triaza-spiro[4.5]dec-(2E)-ylidene]-amide (Example 182);
3,5-Diamino-6-chloro-pyrazine-2-carboxylic acid [8-[3-(4-methyl-thiazol-5-yl)-propionyl]-1,3,8-triaza-spiro[4.5]dec-(2E)-ylidene]-amide (Example 183);
3,5-Diamino-6-chloro-pyrazine-2-carboxylic acid [8-(2-methyl-5-propyl-2H-pyrazole3-carbonyl)-1,3,8-triaza-spiro[4.5]dec-(2E)-ylidene]-amide (Example 184);
3,5-Diamino-6-chloro-pyrazine-2-carboxylic acid [8-[(S)-2-acetylamino-3-(4-isopropoxy-phenyl)-propionyl]-1,3,8-triaza-spiro[4.5]dec-(2E)-ylidene]-amide (Example 185);
3,5-Diamino-6-chloro-pyrazine-2-carboxylic acid [8-[3-(cyclohexyl-methyl-sulfamoyl)-4-methoxy-benzoyl]-1,3,8-triaza-spiro[4.5]dec-(2E)-ylidene]-amide (Example 186);
3,5-Diamino-6-chloro-pyrazine-2-carboxylic acid [8-{2-[4-(3,5-dimethyl-benzenesulfonyl)-piperazin-1-yl]-acetybenzoyl}-1,3,8-triaza-spiro[4.5]dec-(2E)-ylidene]-amide (Example 187);
3,5-Diamino-6-chloro-pyrazine-2-carboxylic acid [8-(3-1H-indol-3-yl-propionyl)-1,3,8-triaza-spiro[4.5]dec-(2E)-ylidene]-amide (Example 188);
3,5-Diamino-6-chloro-pyrazine-2-carboxylic acid [8-{3-[4-(4,6-dimethyl-pyrimidin-2-ylsulfamoyl)-phenylcarbamoyl]-propionyll-1,3,8-triaza-spiro[4.5]dec-(2E)-ylidene]-amide (Example 189);
3,5-Diamino-6-chloro-pyrazine-2-carboxylic acid [8-[3-(2-oxo-5-trifluoromethyl-2H-pyridin-1-yl)-propionyl]-1,3,8-triaza-spiro[4.5]dec-(2E)-ylidene]-amide (Example 191);
3,5-Diamino-6-chloro-pyrazine-2-carboxylic acid [8-[3-(4-sulfamoyl-phenylcarbamoyl)-propionyl]-1,3,8-triaza-spiro[4.5]dec-(2E)-ylidene]-amide (Example 192);
3,5-Diamino-6-chloro-pyrazine-2-carboxylic acid [8-(1-benzyl-5-oxo-pyrrolidine-3-carbonyl)-1,3,8-triaza-spiro[4.5]dec-(2E)-ylidene]-amide (Example 193);
3,5-Diamino-6-chloro-pyrazine-2-carboxylic acid [8-[(R)-2-acetylamino-3-(1H-indol-3-yl)-propionyl]-1,3,8-triaza-spiro[4.5]dec-(2E)-ylidene]-amide Example 194);
3,5-Diamino-6-chloro-pyrazine-2-carboxylic acid [8-[4-(1-benzenesulfonyl-1H-pyrrol3-yl)-4-oxo-butyryl]-1,3,8-triaza-spiro[4.5]dec-(2E)-ylidene-amide (Example 195);
3,5-Diamino-6-chloro-pyrazine-2-carboxylic acid [8-(1-furan-2-ylmethyl-5-oxo-pyrrolidine-3-carbonyl)-1,3,8-triaza-spiro[4.5]dec-(2E)-ylidene]-amide (Example 196);
3,5-Diamino-6-chloro-pyrazine-2-carboxylic acid [8-(6-pyrazol-1-yl-pyridine-3-carbonyl)-1,3,8-triaza-spiro[4.5]dec-(2E)-ylidene]-amide (Example 197);
3,5-Diamino-6-chloro-pyrazine-2-carboxylic acid [8-(3-((R)-1-phenyl-ethylcarbamoyl)-propionyl]-1,3,8-triaza-spiro[4.5]dec-(2E)-ylidene]-amide (Example 198);
3,5-Diamino-6-chloro-pyrazine-2-carboxylic acid [8-[1-(4-chloro-benzyl)-5-oxo-pyrrolidine-3-carbonyl]-1,3,8-triaza-spiro[4.5]dec-(2E)-ylidene]-amide (Example 199);
3,5-Diamino-6-chloro-pyrazine-2-carboxylic acid [8-[2-(3-tert-butyl-isoxazol-5-yl)-acetyl]-1,3,8-triaza-spiro[4.5]dec-(2E)-ylidene]-amide (Example 200);
3,5-Diamino-6-chloro-pyrazine-2-carboxylic acid [8-[6-(2,2,2-trifluoro-ethoxy)-pyridine-3-carbonyl]-1,3,8-triaza-spiro[4.5]dec-(2E)-ylidene]-amide (Example 201);
3,5-Diamino-6-chloro-pyrazine-2-carboxylic acid [8-(4-methyl-2-pyridin-3-yl-thiazole5-carbonyl)-1,3,8-triaza-spiro[4.5]dec-(2E)-ylidene]-amide (Example 202);
3,5-Diamino-6-chloro-pyrazine-2-carboxylic acid [8-(3-pyridin-3-yl-propionyl)-1,3,8-triaza-spiro[4.5]dec-(2E)-ylidene]-amide (Example 203);
3,5-Diamino-6-chloro-pyrazine-2-carboxylic acid [8-(5-dimethylsulfamoyl-2-methyl-furan-3-carbonyl)-1,3,8-triaza-spiro[4.5]dec-(2E)-ylidene]-amide (Example 204);
3,5-Diamino-6-chloro-pyrazine-2-carboxylic acid [8-(1-ethyl-7-methyl-4-oxo-1,4-dihydro-[1,8]naphthyridine-3-carbonyl)-1,3,8-triaza-spiro[4.5]dec-(2E)-ylidene]-amide (Example 205);
3,5-Diamino-6-chloro-pyrazine-2-carboxylic acid [8-(2-pyrazol-1-yl-acetyl)-1,3,8-triaza-spiro[4.5]dec-(2E)-ylidene]-amide (Example 206);
3,5-Diamino-6-chloro-pyrazine-2-carboxylic acid [8-{3-chloro-5-methoxy-4-[2-(4-methyl-piperazin-1-yl)-ethoxy]-benzoybenzoyl}-1,3,8-triaza-spiro[4.5]dec-(2E)-ylidene]-amide (Example 207);
3,5-Diamino-6-chloro-pyrazine-2-carboxylic acid [8-(3-imidazol-1-yl-propionyl)-1,3,8-triaza-spiro[4.5]dec-(2E)-ylidene]-amide (Example 208);
3,5-Diamino-6-chloro-pyrazine-2-carboxylic acid [8-(1-benzyl-1H-imidazole-4-carbonyl)-1,3,8-triaza-spiro[4.5]dec-(2E)-ylidene]-amide (Example 209);
3,5-Diamino-6-chloro-pyrazine-2-carboxylic acid [8-[2-(1,1-dioxo-1lambda*6*-thiomorpholin-4-yl)-3-methyl-butyryl]-1,3,8-triaza-spiro[4.5]dec-(2E)-ylidene]-amide (Example 210);
3,5-Diamino-6-chloro-pyrazine-2-carboxylic acid [8-[4-(toluene-4-sulfonylamino)-butyryl]-1,3,8-triaza-spiro[4.5]dec-(2E)-ylidene]-amide (Example 211);
3,5-Diamino-6-chloro-pyrazine-2-carboxylic acid [8-(1,5-dimethyl-3-oxo-2-phenyl-2,3-dihydro-1H-pyrazole-4-carbonyl)-1,3,8-triaza-spiro[4.5]dec-(2E)-ylidene]-amide (Example 212);
3,5-Diamino-6-chloro-pyrazine-2-carboxylic acid [8-(3-hydroxy-pyridine-2-carbonyl)-1,3,8-triaza-spiro[4.5]dec-(2E)-ylidene]-amide (Example 213);
   are made analogously to Examples 146, 147 or 148 replacing the carboxylic acid reagents with the appropriate carboxylic acids which are all commercially available or prepared as described in section ‘Preparation of Intermediate Compounds’. The compounds are recovered from the reaction mixture and purified using conventional techniques.

### Example 214

### 1-(3-{2-[(E)-3,5-Diamino-6-chloro-pyrazine-2-carbonylimino]-1,3,8-triayrazine-2-carbonylimino]-1,3,8-triazenesulfonyl)-piperidine-3-carboxylic acid

1-(3-{2-[(E)-3,5-Diamino-6-chloro-pyrazine-2-carbonylimino]-1,3,8-triaza-spiro[4.5]decane-8-carbonyl}-benzenesulfonyl)-piperidine-3-carboxylic acid ethyl ester (Example 128) (0.29 g, 0.45 mmol) is dissolved in THF (4 ml) and 2M LiOH (0.22 ml, 0.45 mmol) added. The yellow solution is stirred at room temperature for 5 hours. On concentration in vacuo the resulting sticky yellow solid is ultrasonicated in water (15 ml) until complete dissolution. The pH is adjusted to pH 2 by addition of 1N HCl. The resultant yellow solid is collected by filtration and rinsed with water to yield the title compound; [M+H]⁺620.1.

### Example 215

### 2-[(E)-3,5-Diamino-6-chloro-pyrazine-2-carbonylimino]-1,3,8-triaza-spiro[4.5]decane-8-carboxylic acid benzylamide

To a solution of benzylamine (0.017 ml, 0.154 mmol) in DMF (1 ml) is added 1,1′-carbonyldiimidazole (0.03 g, 0.17 mmol) and the resulting solution is stirred at room temperature for 45 minutes. To this is added 5-Diamino-6-chloro-pyrazine-2-carboxylic acid [1,3,8-triaza-spiro[4.5]dec-(2E)-ylidene]-amide dihydrochloride (Example 38) (0.05 g, 0.15 mmol) and the yellow suspension is stirred for 24 hours. Purification by reverse phase chromatography (Isolute™ C18, 0-100% MeCN in water −0.1% TFA) followed by catch and release resin (SCX-2) eluting with MeOH and 7M NH₃ in MeOH affords the title compound as an off white solid; [M+H]⁺458.1.

### Examples 216-231

These compounds, namely
2-[(E)-3,5-Diamino-6-chloro-pyrazine-2-carbonylimino]-1,3,8-triaza-spiro[4.5]decane-8-carboxylic acid phenylamide (Example 216),
2-[(E)-3,5-Diamino-6-chloro-pyrazine-2-carbonylimino]-1,3,8-triaza-spiro[4.5]decane-8-carboxylic acid [1-(toluene-4-sulfonyl)-1H-indol-5-yl]-amide (Example 217);
2-[(E)-3,5-Diamino-6-chloro-pyrazine-2-carbonylimino]-1,3,8-triaza-spiro[4.5]decane-8-carboxylic acid 3-(4-chloro-phenoxymethyl)-benzylamide (Example 218);
2-[(E)-3,5-Diamino-6-chloro-pyrazine-2-carbonylimino]-1,3,8-triaza-spiro[4.5]decane-8-carboxylic acid [3-(2,4-dichloro-phenyl)-propyl]-amide (Example 219);
2-[(E)-3,5-Diamino-6-chloro-pyrazine-2-carbonylimino]-1,3,8-triaza-spiro[4.5]decane-8-carboxylic acid [2-(3-benzyloxy-phenyl)-ethyl]amide (Example 220);
2-[(E)-3,5-Diamino-6-chloro-pyrazine-2-carbonylimino]-1,3,8-triaza-spiro[4.5]decane-8-carboxylic acid [2-(5,6-dimethyl-1H-indol-3-yl)-ethyl]amide (Example 221);
2-[(E)-3,5-Diamino-6-chloro-pyrazine-2-carbonylimino]-1,3,8-triaza-spiro[4.5]decane-8-carboxylic acid 4-morpholin-4-ylmethyl-benzylamide (Example 222);
2-[(E)-3,5-Diamino-6-chloro-pyrazine-2-carbonylimino]-1,3,8-triaza-spiro[4.5]decane-8-carboxylic acid 3-benzyloxy-benzylamide (Example 223);
2-[(E)-3,5-Diamino-6-chloro-pyrazine-2-carbonylimino]-1,3,8-triaza-spiro[4.5]decane-8-carboxylic acid (2-{442-(4-fluoro-phenyl)-ethoxy]-phenyll-ethyl)-amide (Example 224);
2-[(E)-3,5-Diamino-6-chloro-pyrazine-2-carbonylimino]-1,3,8-triaza-spiro[4.5]decane-8-carboxylic acid [2-(3,5-dimethoxy-phenyl)-ethyl]-amide (Example 225);
2-[(E)-3,5-Diamino-6-chloro-pyrazine-2-carbonylimino]-1,3,8-triaza-spiro[4.5]decane-8-carboxylic acid [3-(4-methoxy-naphthalen-1-yl)-propyl]-amide (Example 226);
2-[(E)-3,5-Diamino-6-chloro-pyrazine-2-carbonylimino]-1,3,8-triaza-spiro[4.5]decane-8-carboxylic acid [2-(4,6-dimethyl-1H-indol-3-yl)-ethyl]amide (Example 227);
2-[(E)-3,5-Diamino-6-chloro-pyrazine-2-carbonylimino]-1,3,8-triaza-spiro[4.5]decane-8-carboxylic acid (3-pyridin-2-yl-propyl)-amide (Example 228);
2-[(E)-3,5-Diamino-6-chloro-pyrazine-2-carbonylimino]-1,3,8-triaza-spiro[4.5]decane-8-carboxylic acid {2-[4-(4-phenyl-butoxy)-phenyl]-ethyl}-amide (Example 229);
2-[(E)-3,5-Diamino-6-chloro-pyrazine-2-carbonylimino]-1,3,8-triaza-spiro[4.5]decane-8-carboxylic acid [2-(4-phenoxy-phenyl)-ethyl]-amide (Example 230);
2-[(E)-3,5-Diamino-6-chloro-pyrazine-2-carbonylimino]-1,3,8-triaza-spiro[4.5]decane-8-carboxylic acid [2-(4-benzyloxy-phenyl)-ethyl]-amide (Example 231);
   are prepared by an analogous procedure to Example 215, replacing benzylamine with the appropriate amines which are either commercially available or synthesized as described in the section ‘Preparation of Intermediate compounds’. The compounds are recovered from reaction mixtures and purified using conventional techniques such as flash chromatography, filtration, recrystallisation and trituration.

### Example 232

### 3,5-Diamino-6-chloro-pyrazine-2-carboxylic acid [8-phenylmethanesulfonyl-1,3,8-triaza-spiro[4.5]dec-(2E)-ylidene]-amide

To a solution of 5-Diamino-6-chloro-pyrazine-2-carboxylic acid [1,3,8-triaza-spiro[4.5]dec-(2E)-ylidene]-amide dihydrochloride (Example 38) (0.05 g, 0.15 mmol) in DMF (2 ml) is added alpha-toluenesulfonyl chloride (0.04 g, 0.20 mmol) and triethylamine (0.02 ml, 0.15 mmol) and the yellow solution is stirred at room temperature for 2 hours. Purification by reverse phase chromatography (Isolute™ C18, 0-100% MeCN in water −0.1% TFA) followed by catch and release resin (SCX-2) eluting with MeOH and 7M NH₃ in MeOH affords the title compound as a yellow solid; [M+H]⁺478.98.

### Examples 233-245

The following compounds, namely
3,5-Diamino-6-chloro-pyrazine-2-carboxylic acid [8-benzenesulfonyl-1,3,8-triaza-spiro[4.5]dec-(2E)-ylidene]-amide (Example 233);
3,5-Diamino-6-chloro-pyrazine-2-carboxylic acid [8-(1-methyl-1H-indole-4-sulfonyl)-1,3,8-triaza-spiro[4.5]dec-(2E)-ylidene]-amide (Example 234);
3,5-Diamino-6-chloro-pyrazine-2-carboxylic acid [8-(1-methyl-1H-indole-5-sulfonyl)-1,3,8-triaza-spiro[4.5]dec-(2E)-ylidene]-amide (Example 235);
3,5-Diamino-6-chloro-pyrazine-2-carboxylic acid [8-(7-chloro-benzo[1,2,5]oxadiazole-4-sulfonyl)-1,3,8-triaza-spiro[4.5]dec-(2E)-ylidene]-amide (Example 236);
3,5-Diamino-6-chloro-pyrazine-2-carboxylic acid [8-(2-phenyl-ethanesulfonyl)-1,3,8-triaza-spiro[4.5]dec-(2E)-ylidene]-amide (Example 237);
3,5-Diamino-6-chloro-pyrazine-2-carboxylic acid [8-[4-(5-methyl-2-phenyl-oxazol-4-ylmethoxy)-benzenesulfonyl]-1,3,8-triaza-spiro[4.5]dec-(2E)-ylidene]-amide (Example 238);
3,5-Diamino-6-chloro-pyrazine-2-carboxylic acid [8-(4-phenyl-5-trifluoromethyl-thiophene-3-sulfonyl)-1,3,8-triaza-spiro[4.5]dec-(2E)-ylidene]-amide (Example 239);
3,5-Diamino-6-chloro-pyrazine-2-carboxylic acid [8-(5-cyano-2-methoxy-benzenesulfonyl)-1,3,8-triaza-spiro[4.5]dec-(2E)-ylidene]-amide (Example 240);
3,5-Diamino-6-chloro-pyrazine-2-carboxylic acid [8-[2-(4-chloro-phenyl)-ethanesulfonyl]-1,3,8-triaza-spiro[4.5]dec-(2E)-ylidene]-amide (Example 241);
3,5-Diamino-6-chloro-pyrazine-2-carboxylic acid [8-(2-phenyl-3H-benzoimidazole-5-sulfonyl)-1,3,8-triaza-spiro[4.5]dec-(2E)-ylidene]-amide (Example 242);
3,5-Diamino-6-chloro-pyrazine-2-carboxylic acid [8-[2-(2-chloro-phenyl)-ethanesulfonyl]-1,3,8-triaza-spiro[4.5]dec-(2E)-ylidene]-amide (Example 243);
3,5-Diamino-6-chloro-pyrazine-2-carboxylic acid [8-[2-(2,2,2-trifluoro-acetyl)-1,2,3,4-tetrahydro-isoquinoline-7-sulfonyl]-1,3,8-triaza-spiro[4.5]dec-(2E)-ylidene]-amide (Example 244);
3,5-Diamino-6-chloro-pyrazine-2-carboxylic acid [8-[2-(3-chloro-phenyl)-ethanesulfonyl]-1,3,8-triaza-spiro[4.5]dec-(2E)-ylidene]amide (Example 245);
   are prepared by an analogous procedure to Example 232, replacing alpha-toluenesulfonyl chloride with the appropriate sulfonyl chlorides which are either commercially available or synthesized as described in the section ‘Preparation of Intermediate compounds’. The compounds are recovered from reaction mixtures and purified using conventional techniques such as flash chromatography, filtration, recrystallisation and trituration.

### Example 246

### 3,5-Diamino-6-chloro-pyrazine-2-carboxylic acid [8-(1-phenyl-ethyl)-1,3,8-triaza-spiro[4.5]dec-(2E)-ylidene]-amide

A mixture of 1-(3,5-diamino-6-chloro-pyrazine-2-carbonyl)-2-methyl-isothiourea (Intermediate A) (1.7 g, 4.54 mmol) and 4-aminomethyl-1-(1-phenyl-ethyl)-piperidin-4-ylamine (Intermediate BM) (1.6 g, 4.59 mmol) in propan-2-ol (50 ml) is stirred at 80° C. for 16 hours. The reaction mixture is concentrated in vacuo and purified by column chromatography (basic alumina, 0-2% MeOH in DCM) to obtain pale yellow solid. The compound obtained is further dissolved in MeOH and precipitated by adding diethyl ether. The supernatant solvent mixture is decanted and the product is washed again with diethyl ether and dried under vacuum to afford the title compound as off-white solid; [M+H]⁺429.

### Example 247

### 3,5-Diamino-6-chloro-pyrazine-2-carboxylic acid [8-(4-methoxy-benzyl)-1,3,8-triaza-spiro[4.5]dec-(2E)-ylidene]-amide

This compound is prepared analogously to Example 246 by replacing 4-aminomethyl-1-(1-phenyl-ethyl)-piperidin-4-ylamine (Intermediate BM) with 4-aminomethyl-1-(4-methoxy-benzyl)-piperidin-4-ylamine (Intermediate BN) [M+H]⁺445.

### Example 248

### 3,5-Diamino-6-chloro-pyrazine-2-carboxylic acid [8-pyridin-4-ylmethyl-1,3,8-triaza-spiro[4.5]dec-(2E)-ylidene]-amide

This compound is prepared analogously to Example 246 by replacing 4-aminomethyl-1-(1-phenyl-ethyl)-piperidin-4-ylamine (Intermediate BM) with 4-aminomethyl-1-pyridin-4-ylmethyl-piperidin-4-ylamine (Intermediate BO); [M+H]⁺=416.

### Example 249

### 3,5-Diamino-6-chloro-pyrazine-2-3carboxylic acid [8-(3-phenyl-propyl)-1,3,8-triaza-spiro[4.5]dec-(2E)-ylidene]-amide

This compound is prepared analogously to Example 246 by replacing 4-aminomethyl-1-(1-phenyl-ethyl)-piperidin-4-ylamine (Intermediate BM) with 4-aminomethyl-1-(3-phenyl-propyl)-piperidin-4-ylamine (Intermediate BP) [M+H]⁺443.

### Example 250

### 3,5-Diamino-6-chloro-pyrazine-2-carboxylic acid [8-cyclohexylmethyl-1,3,8-triaza-spiro [4.5]dec-(2E)-ylidene]-amide

This compound is prepared analogously to Example 246 by replacing 4-aminomethyl-1-(1-phenyl-ethyl)-piperidin-4-ylamine (Intermediate BM) with 4-aminomethyl-1-cyclohexylmethyl-piperidin-4-ylamine (Intermediate BQ) [M+H]⁺421.

### Example 251

### (E)-tert-Butyl 2′-(3,5-diamino-6-chloropyrazine-2-carbonylimino)-8-azaspiro[bicyclo [3.2.1]octane-3,4′-imidazolidine]-8-carboxylate

This compound is prepared analogously to Example 246 by replacing 4-aminomethyl-1-(1-phenyl-ethyl)-piperidin-4-ylamine (Intermediate BM) with 3-amino-3-aminomethyl-8-aza-bicyclo [3.2.1]octane-8-carboxylic acid tert-butyl ester (Intermediate BR) [M+H]⁺451.

### Example 252

### (E)-N-(8-(1H-indole-4-carbonyl)-8-azaspiro[bicyclo[3.2.1]octane-3,4′-imidazolidine]-2′-ylidene)-3,5-diamino-6-chloropyrazine-2-carboxamide

### Step 1

Iodotrimethylsilane (0.23 ml, 1.66 mmol) is added to a suspension of (E)-tert-Butyl 2′-(3,5-diamino-6-chloropyrazine-2-carbonylimino)-8-azaspiro[bicyclo[3.2.1]octane-3,4′-imidazolidine]-8-carboxylate (Ex. 251) (500 mg, 1.11 mmol) in DCM (10 ml). DMF (5 ml) is then added and the reaction is stirred at room temperature overnight. Iodotrimethylsilane (0.5 ml) is added and the reaction mixture is concentrated in vacuo. The yellow solid is suspended in DCM and collected by filtration. The solid is dissolved in 1:1 MeOH/DCM and loaded onto an SCX-2 cartridge eluted with DCM followed by MeOH and NH₃/MeOH. The methanolic ammonia fractions are concentrated in vacuo to afford (E)-3,5-diamino-6-chloro-N-(8-azaspiro[bicyclo[3.2.1]octane-3,4′-imidazolidine]-2′-ylidene)pyrazine-2-carboxamide as a yellow gum; [M+H]⁺351.

### Step 2

(E)-3,5-diamino-6-chloro-N-(8-azaspiro[bicyclo[3.2.1]octane-3,4′-imidazolidine]-2′-ylidene)pyrazine-2-carboxamide (170 mg, 0.49 mmol) is dissolved in DMF (10 ml) along with HATU (184 mg, 0.49 mmol) and 4-indole-carboxylic acid (78 mg, 0.49 mmol). N-Methyl morpholine (160 ml, 1.45 mmol) is added and the solution stirred at room temperature overnight. The mixture is then concentrated in vacuo. EtOAc (100 ml) is added and the solution washed with water (100 ml). The organic phase is dried (MgSO₄) and concentrated in vacuo. Purification by flash chromatography (SiO₂, DCM/MeOH) gives the title compound as a yellow solid; [M+H]⁺494.15, 496.27 for Cl isotopes.

### Example 253

### (E)-3,5-diamino-N-(8-(3-(4-(benzyloxy)phenyl)propanoyl)-8-azaspiro[bicyclo[3.2.1]octane-3,4′-imidazolidine]-2′-ylidene)-6-chloropyrazine-2-carboxamide

(E)-3,5-diamino-6-chloro-N-(8-azaspiro[bicyclo[3.2.1]octane-3,4′-imidazolidine]-2′-ylidene)pyrazine-2-carboxamide (prepared as described for Ex. 252) (280 mg, 0.798 mmol) is dissolved in DMF (8 ml) along with HATU (303 mg, 0.798 mmol) and 3-(4-benzyloxy-phenyl)-propionic acid (205 mg, 0.798 mmol). N-Methyl morpholine (0.263 ml, 2.394 mmol) is added and the solution stirred at room temperature for 6 hours. The mixture is then concentrated in vacuo. EtOAc (100 ml) is added and the solution washed with water (100 ml). The organic phase is dried (MgSO₄) and concentrated. The residue is dissolved in MeOH (20 ml) and dry loaded onto silica (5 g). Purification by flash chromatography (SiO₂, DCM/MeOH) gives the title compound as a tan solid; [M+H]⁺589.20, 591.19 for Cl isotopes.

### Preparation of Intermediate Compounds

### Intermediate A

### 1-(3,5-Diamino-6-chloro-pyrazine-2-carbonyl)-2-methyl-isothiourea hydroiodide

### Method 1

This compound is prepared according to Cragoe, Edward J., Jr.; Woltersdorf, Otto W., Jr.; De Solms, Susan Jane. Heterocyclic-substituted pyrazinoylguanidines, and a pharmaceutical composition containing them. EP 17152 Page 4

### Method 2

### Step 1

A stirred suspension of 3,5-diamino-6-chloro-pyrazine-2-carboxylic acid methyl ester (110 g, 542.9 mmol) in MeOH (500 ml) at 5-10° C. (ice-bath) is treated dropwise with a suspension of lithium hydroxide (46.6 g, benzoyl}benzoyl} mmol) in water (500 ml). The reaction mixture is heated to 50° C. for 5 hours then cooled to room temperature and stirred overnight. The resulting precipitate is collected by filtration and dried in a vacuum oven to afford Lithium 3,5-diamino-6-chloro-pyrazine-2-carboxylic acid as the lithium salt (di-hydrate); [M−Li]⁻187.

### Step 2

A stirred suspension of S-methyl-iso-thiourea sulphate (10 g, 35.9 mmol) in toluene (75 ml) is treated with 4 M NaOH (15 ml) at room temperature. To the two-phase mixture is added di-tert butyl dicarbonate (3.27 g, 15 mmol) in one portion. The reaction mixture is stirred at room temperature for 1 hour, then heated to 60° C. overnight. The organic portion is separated, washed with brine solution, then dried over Na₂SO₄, filtered and concentrated in vacuo to a viscous oil, which crystallized under high vacuum to afford tert-Butyl amino(methylthio)methylenecarbamate as a colorless solid.

### Step 3

A stirring suspension of lithium 3,5-diamino-6-chloro-pyrazine-2-carboxylic acid (22.6 g, 98.03 mmol) in DMF (400 ml) is treated portionwise with HATU (41 g, 107.83 mmol), under an inert atmosphere of nitrogen. The reaction mixture is stirred at room temperature for 2 hours and then tert-butyl amino(methylthio)methylenecarbamate (20.5 g, 107.83 mmol) is added portion wise over a period of 10 minutes. The reaction mixture is stirred at room temperature for a further 1.5 hours then heated to 50° C. and stirred overnight. The resulting precipitate is hot filtered, washing with water and dried in a vacuum oven (40° C.) overnight to afford tert-Butyl (3,5-diamino-6-chloropyrazine-2-carboxamido)(methylthio) methylene carbamate; [M+H]⁺361.

### Step 4

tert-Butyl (3,5-diamino-6-chloropyrazine-2-carboxamido)(methylthio)methylene carbamate (50 g, 139 mmol) is slurried in DCM (500 ml). TFA (53.4 ml, 693 mmol) is dissolved in DCM (100 ml) and added dropwise over 45 mins to form a brown solution. The solution is stirred at room temperature overnight, after which time a yellow precipitate has formed. The solid is collected by filtration, and dried in vacuo to yield the title compound; [M+H]⁺261.1.

### Intermediate B

### ((S)-5,6-Diamino-hexyl)-carbamic acid benzyl ester

### Step 1

A solution of BOC-lysinol-(Z)—OH (0.5 g, 1.36 mmol) in dry THF (1 ml) under an inert atmosphere of argon is treated with PS-triphenylphosphine (0.91 g, 3.00 mmol/g loading). To this mixture is added phthalimide (0.2 g, 1.36 mmol) and DEAD (0.24 ml, 1.50 mmol) in dry THF (4 ml) and the reaction mixture is stirred at room temperature overnight. The resin is removed by filtration under vacuum and the filtrate is concentrated in vacuo. Purification of the crude white solid by chromatography on silica eluting with 20-50% EtOAc in iso-hexane (1% TEA) affords [(S)-5-Benzyloxycarbonylamino-1-(1,3-dioxo-1,3-dihydro-isoindol-2-ylmethyl)-pentybenzoyl}-carbamic acid tert-butyl ester as a white crystalline solid; [M+H]⁺496.

### Step 2

A solution of [(S)-5-benzyloxycarbonylamino-1-(1,3-dioxo-1,3-dihydro-isoindol-2-ylmethyl)-pentyl]-carbamic acid tert-butyl ester (0.63 g, 1.27 mmol) in DCM (5.1 ml) and EtOH (5.1 ml) is treated with hydrazine hydrate (0.318 g, 6.35 mmol) and the reaction mixture is stirred at room temperature overnight. A white precipitate forms which is removed by filtration and washed with DCM (3×10 ml). The filtrate is concentrated in vacuo and redissolved in DCM (15 ml) and MeOH (2 ml). Undissolved material is removed by filtration and the filtrate is concentrated in vacuo. The resulting oily yellow solid is purified by chromatography on silica eluting with 10-50% MeOH in DCM (1% TEA) to afford ((S)-1-Aminomethyl-5-benzyloxycarbonylamino-pentyl)-carbamic acid tert-butyl ester as a clear oil; [M+H]⁺366.

### Step 3

A solution of ((S)-1-aminomethyl-5-benzyloxycarbonylamino-pentyl)-carbamic acid tert-butyl ester (0.24 g, 0.657 mmol) in DCM (2.4 ml) is treated dropwise with TFA (0.6 ml) and stirred at room temperature for 3 days. The solvent is removed in vacuo to afford ((S)-5,6-Diamino-hexyl)-carbamic acid benzyl ester as a yellow oil; [M+H]⁺266.

### Intermediate C

A mixture of 4-[4-(2-amino-ethylamino)-butyl]-phenol and N*1*-[4-(4-methoxy-phenyl)-butyl]ethane-1,2-diamine

### Step 1

A solution of 4-methoxyphenylbutryric acid (6.99 g, 36 mmol) in THF (70 ml) is treated with EDCI (7.6 g, 36.9 mmol) followed by N-ethylmorpholine (9.2 ml, 72 mmol). After stirring at room temperature for 1 hour, N—BOC-ethylene diamine (5.84 g, 36 mmol) is added and the resulting mixture is stirred at room temperature overnight. The reaction is quenched by addition of saturated sodium hydrogen carbonate solution and extracted with EtOAc. The organic portion is washed with citric acid solution, brine, dried (MgSO₄) and concentrated in vacuo until 25 ml of solvent remained. The suspension is filtered to afford {2-[4-(4-Methoxy-phenyl)-butyrylamino]-ethyl}-carbamic acid tert-butyl ester: as a white solid.

### Step 2

A solution of {2-[4-(4-methoxy-phenyl)-butyrylamino]-ethyl}-carbamic acid tert-butyl ester (6 g, 17.88 mmol) in dry THF (60 ml) under an inert atmosphere of Argon is treated carefully with borane. THF complex (53.88 ml, 1M Borane in THF). The reaction mixture is heated at reflux for 2 hours and then allowed to cool to room temperature overnight. The mixture is quenched by addition of MeOH and then heated to 70° C. for a further 2 hours. After cooling to room temperature, the solvent is removed in vacuo to afford {2-[4-(4-Methoxy-phenyl)-butylamino]-ethyl}-carbamic acid tert-butyl ester as a viscous oil; [M+H]⁺323.

### Step 3

A suspension of {2-[4-(4-methoxy-phenyl)-butylamino]-ethyl}-carbamic acid tert-butyl ester (5.85 g, 18.1 mmol) in HBr (30 ml of a 48% solution) is heated at reflux for 2 hours. After cooling to room temperature, the solvent is removed in vacuo. The crude residue is suspended in EtOAc and filtered to afford a solid which consisted of a mixture of 4-[4-(2-amino-ethylamino)-butyl]-phenol and N*1*-14-(4-methoxy-phenyl)-butyl]-ethane-1,2-diamine in approximately 1:1 ratio; [M+H]⁺209 and 223.

### Intermediate D

### (S)-3-(4-methoxy-phenyl)-propane-1,2 diamine

(S)-2-Amino-3-(4-methoxy-phenyl)-propionamide is prepared according to the procedure described on page 3880, Method 2.1.3 of Journal of Physical Chemistry B, 108(12), 3879-3889; 2004 and is reduced analogously to Intermediate C.1-(3,4-Dichloro-phenyl)-ethane-1,2-diamine This compound is prepared according to the procedure described on page 907, Method 5 in the Journal of Medicinal Chemistry (1973), 16(8), 901-8.

### Intermediate F

### 4,5-Diaminopentanoic acid dihydrochloride

This compound is prepared according to the procedure described in ‘Radiolabeling chelating compounds comprising sulfur atoms, with metal radionuclides.’ EP 300431 page 12, Intermediate 3.

### Intermediate G

### 4-Amino-1-benzyl-piperidine-4-carbonitrile

Step 1 To a solution of ammonium chloride (1.73 g, 32.3 mmol) in water (20 ml) is added a 30% ammonia solution (2 ml) followed by 1-benzyl-4-piperidone. After 20 minutes sodium cyanide (1.47 g, 30 mmol) is added portionwise over 15 minutes. After stirring for one hour, water (50 ml) is added and the products are extracted with DCM (3×50 ml), dried (MgSO₄) filtered and concentrated in vacuo. Purification by chromatography on silica eluting with 50-100% EtOAc in iso-hexane affords 4-Aminomethyl-1-benzyl-piperidine-4-ylamine; [M+H]⁺216

### Step 2

To a solution of lithium aluminum hydride (1 M in THF, 10.4 ml) in dry diethyl ether (15 ml), cooled to 0° C., under an argon atmosphere is added dropwise 4-amino methyl-1-benzyl-piperidine-4-ylamine (900 mg, 4.18 mmol) in dry diethyl ether (15 ml). The reaction mixture is heated at reflux for 24 h and then cooled to 0° C. Water (0.25 ml) is added followed by a 15% aqueous NaOH (0.25 ml) and then water (0.7 ml). After warming to room temperature MgSO₄ (150 mg) is added and stirred for 15 minutes. The solids are removed by suction filtration and the filtrate evaporated to give an oil. The solids are extracted with refluxing diethyl ether (80 ml) using a Soxhlet extractor for 14 hours. The diethyl ether is removed in vacuo and the two oils combined and purified by chromatography on silica eluting with 10-25% 2M ammonia in methanol solution in dichloromethane to give 4-Amino-1-benzyl-piperidine-4-carbonitrile; [M+H]⁺220

### Intermediate H

### 5-14-((R)-2,2-Dimethyl-[1,3]-dioxolane-4-ylmethoxy)-phenybenzoyl}-pentane-1,2-diamine

Step 1

To 3-(4-hydroxyphenyl)-1-propanol (10 g, 66 mmol) and potassium carbonate (13.5 g, 100 mmol) in acetone (200 ml) is added (S)-glycidol (6.5 ml, 100 mmol). The mixture is heated at reflux for 18 hours. After cooling to room temperature the solvent is removed in vacuo and the residue partitioned between EtOAc and water. The aqueous layer is further extracted twice with EtOAc and the combined organic portions are washed with water, brine, dried (MgSO₄), filtered and concentrated in vacuo. The crude residue is purified by flash column chromatography on silica eluting with 1:1 EtOAc/iso-hexane to afford (S)-3-[4-(3-Hydroxy-propyl)-phenoxy]-propane-1,2-diol as a white solid; ¹H NMR (CDCl₃): 1.20 (1H, br), 1.85 (2H, pent, J=6.8 Hz), 1.98 (1H, br), 2.58 (1H, br), 2.65 (2H, tr, J=6.9 Hz), 3.56 (2H, tr, J=6.8 Hz), 3.72 (1H, m), 3.83 (1H, m), 4.00 (2H, dd, J=2.1 Hz, J=6.5 Hz), 4.09 (1H, br), 6.82 (2H, d, J=7.4 Hz), 7.10 (2H, d, J=7.4 Hz).

Step 2

To (S)-3-[4-(3-hydroxy-propyl)-phenoxy]-propane-1,2-diol (benzoyl} 0.5 g, 50.9 mmol) in dry DMF (150 ml) is added pyridinium p-toluenesulfonate (1.28 g, 5 mmol) and 2,2-dimethoxypropane (31 ml, 250 mmol). The mixture is stirred at room temperature for 18 hours and then the solvent is removed in vacuo. The residue is dissolved in EtOAc (150 ml) and washed with water, saturated aqueous sodium hydrogen carbonate solution, brine, dried (MgSO₄) and concentrated in vacuo. The residue is purified by chromatography on silica eluting with 1:4 EtOAc/iso-hexane to 1:1 EtOAc/iso-hexane to afford (3-[4-((R)-2,2-Dimethyl-[1,3]-dioxolan-4-ylmethoxy)-phenyl]-propan-1-ol as a colorless oil; 1H NMR (CDCl3): 1.25 (1H, br), 1.39 (3H, s), 1.43 (3H, s), 1.85 (2H, pent, J=6.9 Hz), 2.63 (2H, tr, J=6.9 Hz), 3.63 (2H, tr, J=6.9 Hz), 3.90 (2H, m), 4.02 (1H, m), 4.12 (1H, m), 4.50 (1H, pent, J=6.8 Hz), 6.82 (2H, d, J=7.4 Hz), 7.10 (2H, d, J=7.4 Hz).

Step 3

To (3-[4-((R)-2,2-dimethyl-[1,3]dioxolan-4-ylmethoxy)-phenyl]-propan-1-ol (12.2 g, 46 mmol) in dry ether (150 ml) is added TEA (12.8 ml, 92 mmol). The mixture is cooled to 0° C. and treated dropwise with methanesulfonyl chloride (5.3 ml, 69 mmol). The reaction mixture is allowed to warm to room temperature and then stirring continued for 3 hours. The resulting mixture is washed with water (2×100 ml), saturated aqueous sodium hydrogencarbonate, brine, dried (MgSO4) and concentrated in vacuo to give Methanesulfonic acid 3-[4-((R)-2,2-dimethyl[1,3]dioxolan-4-ylmethoxy)-phenyl]-propylester as a white solid; 1H NMR (CDCl3): 1.39 (3H, s), 1.43 (3H, s), 2.02 (2H, pent, J=6.9 Hz), 2.63 (2H, tr, J=6.9 Hz), 3.00 (3H, s), 3.90 (2H, m), 4.05 (1H, m), 4.14 (3 h, m), 4.46 (1H, pent, J=6.8 Hz), 6.82 (2H, d, J=7.4 Hz), 7.10 (2H, d, J=7.4 Hz).

Step 4

Methanesulfonic acid 3-[4-((R)-2,2-dimethyl[1,3]dioxolan-4-ylmethoxy)-phenyl]-propylester (11.8 g, 34.2 mmol) in acetone (200 ml) is treated with lithium bromide (8.9 g, 100 mmol) and then heated at reflux for 5 h. After cooling to room temperature, the mixture is concentrated in vacuo. The resulting residue is dissolved in EtOAc (150 ml), washed with water (2×50 ml), brine, dried (MgSO₄), filtered and concentrated in vacuo to give an oil. Purification by chromatography on silica eluting with 4:1 iso-hexane/EtOAc gives (R)-4-[4-(3-Bromo-propyl)-phenoxymethyl]-2,2-dimethyl-[1,3]dioxolane as a colorless oil which solidifies; 1H NMR (CDCl3): 1.39 (3H, s), 1.43 (3H, s), 2.02 (2H, pent, J=6.9 Hz), 2.63 (2H, tr, J=6.9 Hz), 3.38 (2H, tr, J=6.9 Hz), 3.90 (2H, m), 4.02 (1H, m), 4.15 (1H, m), 4.46 (1H, pent, J=6.9 Hz), 6.82 (2H, d, J=7.4 Hz), 7.10 (2H, d, J=7.4 Hz).

Step 5

A solution of N-(diphenylmethylene)aminoacetonitrile (5.14 g, 23.4 mmol) in DCM (12 ml) is treated with (R)-4-[4-(3-bromo-propyl)-phenoxymethyl]-2,2-dimethyl-[1,3]dioxolane (8.1 g, 24 mmol) in DCM (12 ml) and cooled to 0° C. 48% aqueous NaOH (20 ml) is added followed by benzyltriethylammonium chloride (530 mg, 2.4 mmol) and the resulting mixture is allowed to warm to room temperature. After stirring vigorously for 4 hours mixture is diluted with DCM (100 ml) and the aqueous portion is removed. The organic layer is washed with water (2×50 ml), brine, dried (MgSO4), filtered and concentrated in vacuo. The crude product is purified by chromatography on silica eluting with 15:1 iso-hexane/diethyl ether to 4:1 iso-hexane/diethyl ether to yield 2-(Benzhydrylidene-amino)-544-((R)-2,2-dimethyl-[1,3]dioxolan-4-ylmethoxy)-phenyl]pentanenitrile as a yellow oil; 1H NMR (CDCl3): mix of diastereoisomers 1.39 (3H, s), 1.43 (3H, s), 1.71 (2H, m), 1.80-1.98 (2H, m), 2.52 (2H, tr, J=7.0 Hz) 3.90, (2H, m), 4.02 (1H, m), 4.10-4.22 (2H, m), 4.47 (1H, pent, J=6.9 Hz), 6.82 (2H, d, J=7.4 Hz), 7.05 (2H, d, J=7.4 Hz), 7.19 (2H, m), 7.35 (2H, tr, J=7.2 Hz), 7.40-7.50 (4H, m), 7.60 (2H, d, J=7.1 Hz).

Step 6

To a solution of 2-(benzhydrylidene-amino)-5-[4-((R)-2,2-dimethyl-[1,3]dioxolan-4-ylmethoxy)-phenyl]pentanenitrile (7.2 g, 15.5 mmol) in THF (50 ml) is added a 2M HCl (aq) (5 ml). The solution is heated at 40° C. for 4 hours and then allowed to cool to room temperature. The pH is adjusted to pH 9-10 using saturated aqueous sodium hydrogen carbonate solution and the organic solvent is removed in vacuo. The crude residue is dissolved in EtOAc (100 ml) and washed with water, brine, dried (MgSO4), filtered and concentrated in vacuo. The resulting residue is purified by chromatography on silica eluting with 5:1 to 1:1 iso-hexane/ethyl aEtOAc and 1% triethylamine to yield 2-Amino-5-[4-((R)-2,2-dimethyl-[1,3]dioxolan-4-ylmethoxy)-phenyl]-pentanenitrile as a colorless oil which solidifies; 1H NMR (CDCl3): mixture of diastereoisomers 1.39 (3H, s), 1.43 (3H, s), 1.70-1.87 (4H, m), 2.60 (2H, tr, J=7.1 Hz), 3.62 (1H, br), 3.90 (2H, m), 4.00-4.18 (2H, m), 4.48 (1H, pent, J=6.9 Hz), 6.82 (2H, d, J=7.4 Hz), 7.10 (2H, d, J=7.4 Hz). [M+H]+305.

Step 7

A solution of 2-amino-5-[4-((R)-2,2-dimethyl-[1,3]dioxolan-4-ylmethoxy)-phenyl]-pentanenitrile (1.7 g, 4.28 mmol) in a 2M ammonia in methanol solution (50 ml) is passed through a H-CUBE apparatus fitted with a Raney nickel CatCart at 50° C. and a hydrogen pressure of 50 bar and a flow rate of 1.5 ml/min. After 5 hours of continuous cycling of the solution the reaction mixture is concentrated in vacuo to give 5-[4-((R)-2,2-Dimethyl-[1,3]dioxolane-4-ylmethoxy)-phenyl]-pentane-1,2-diamine as a light-yellow oil; [M+H]+309.

### Intermediate I

### 5-(4-Methoxy-phenyl)-pentane-1,2-diamine

This compound is prepared analogously to Intermediate H by replacing (3-[4-((R)-2,2-dimethyl-1,3]dioxolan-4-ylmethoxy)-phenyl]-propan-1-ol with 4-(4-methoxyphenyl)-1-butanol.

### Intermediate J

### 1-Aminomethyl-cyclopentylamine

Step 1

To a cooled 0° C. solution of (1-cyano-cyclopentyl)-carbamic acid tert-butyl ester (430 mg, 2.04 mmol) in dry THF (4.3 ml) under an atmosphere of argon is added dropwise 1.0 M LiAlH4 (6.13 ml, 6.13 mmol). The reaction mixture is allowed to warm to room temperature and stirred for 3.5 hours. The mixture is then re-cooled to 0° C. and cautiously quenched with water (0.4 ml): 15% NaOH (0.8 ml): water (1.2 ml) (1:2:3 eq). The resultant mixture is filtered through Celite® (filter material) to remove the inorganic solids and rinsed with MeOH. The filtrate is concentrated in vacuo, to yield a white solid, which is purified by chromatography on silica eluting with 30% MeOH in DCM to afford (1-Aminomethyl-cyclopentyl)-carbamic acid tert-butyl ester; [M+H]+215.

Step 2

Iodotrimethylsilane (0.091 ml, 0.67 mmol) is added dropwise to a solution of (1-aminomethyl-cyclopentyl)-carbamic acid tert-butyl ester (120 mg, 0.56 mmol) in DCM (2.4 ml) and left to stir overnight. The resulting suspension is quenched with MeOH (2.4 ml) and concentrated in vacuo to yield 1-Aminomethyl-cyclopentylamine as a dark oil, which is used without further purification.

### Intermediate K

### (4-((R)-4,5-Diamino-pentyl)-phenol

Steps 1 and 2

(R)-2-tert-Butoxycarbonylamino-5-(4-tert-butoxy-phenyl)-pentanoic acid ethyl ester is prepared according to the procedure of Ding, Chuanyong.; Ma, Rujian.; Rong, Guobin. Preparation of w-Phenyl-(2S)—N-Boc-amino Acid Ethyl esters; Chinese Journal of Organic Chemistry Vol 26(12) 2006, 1694 &1695, replacing Ethyl Boc-L-pyroglutamate with Ethyl Boc-D-pyroglutamate & Bromomethyl-benzene with 1-Bromo-4-tert-butoxy-benzene in Example 2a, using preparation steps 2.2, 2.3, and 2.5; [M+H]+394.

Step 3

(R)-2-tert-Butoxycarbonylamino-5-(4-tert-butoxy-phenyl)-pentanoic acid ethyl ester (179 g, 460 mmol) is dissolved in 7M NH3 in MeOH (400 ml, 2800 mmol) and stirred at room temperature for 4 days. The reaction is concentrated in vacuo keeping the temperature below 30° C. to afford [(R)-4-(4-tert-Butoxy-phenyl)-1-carbamoyl-butyl]-carbamic acid tert-butyl ester [M+H]+364.

Step 4

A solution of [(R)-4-(4-tert-Butoxy-phenyl)-1-carbamoyl-butyl]-carbamic acid tertbutyl ester (167 g, 458 mmol) in 1 M HCl in Et2O (4000 ml) is stirred at room temperature for 3 days. After this time, a white solid forms which is collected by filtration and washed with Et2O to yield (R)-2-Amino-5-(4-hydroxy-phenyl)-pentanoic acid amide; [M+H]+209.

Step 5

To a stirred solution of (R)-2-Amino-5-(4-hydroxy-phenyl)-pentanoic acid amide (5 g, 24.01 mmol) in THF (250 ml) is added imidazole (4.90 g, 72 mmol), followed by tert-butyldimethylchlorosilane (3.98 g, 26.4 mmol). The resulting solution is heated at 70° C. for 4 hours and then allowed to cool to room temperature. Dilution with Et2O (200 ml) washing with water (2×100 ml) and brine (100 ml), drying MgSO4, and concentration in vacuo yields (R)-2-Amino-5-[4-(tert-butyl-dimethyl-silanyloxy)-phenyl]-pentanoic acid amide; [M+H]+323.

Step 6

A solution of (R)-2-Amino-5[4-(tert-butyl-dimethyl-silanyloxy)-phenyl]-pentanoic acid amide (7.74 g, 24 mmol) in THF is stirred at 5° C. and borane (96 ml of a 1 M solution in THF, 96 mmol) is added. The mixture is stirred at 5° C. until a homogeneous mixture is obtained and then stirred at room temperature for 30 minutes and 35° C. for 3 hours. After this time, further borane (24 ml of a 1 M solution in THF, 24 mmol) is added and the reaction is heated at 35° C. for 18 hours. After this time, a further portion of borane (24 ml of a 1 M solution in THF, 24 mmol) is added and the reaction heated at 35° C. for a further 24 hours. After this time, the reaction is cooled to 10° C., and quenched by adding dropwise to MeOH (50 ml) at −5° C. After allowing to warm to room temperature the solvent is removed in vacuo to afford a yellow oil. The oil is dissolved in MeOH (250 ml) and SCX-2 silica (180 g, 0.63 mmol/g, 120 mmol) is added. The silica suspension is shaken for 18 hours, the silica is removed by filtration, washed with MeOH (3×100 ml), then suspended in 7M NH3 in MeOH and shaken for 18 hours. The silica is removed by filtration and the 7M NH3 in MeOH is removed in vacuo to afford the title compound as a yellow oil; [M+H]+195.

### Intermediate L

### 4-((S)-4,5-Diamino-pentyl)-phenol

This compound is prepared analogously to Intermediate K (NVP-QBM333), replacing Ethyl Boc-D-pyroglutamate in step 1 with Ethyl Boc-L-pyroglutamate; [M+H]+195.

### Intermediate M

### (R)-tert-butyl 5-(4-hydroxyphenyl)pentane-1,2-diyldicarbamate

To a solution of (4-((R)-4,5-Diamino-pentyl)-phenol (Intermediate K) (775 mg, 1.99 mmol) in DCM (10 ml) is added triethylamine (1.14 ml, 8.08 mmol) and a solution of di-tert-butyl dicarbonate (1.33 g, 6.08 mmol) in DCM (10 ml) and the resulting solution is stirred at room temperature for 18 hours. The solvent is removed in vacuo and the residue purified by chromatography (SiO2, EtOAc/iso-hexane) to afford the title compound; [M+H]+395.

### Intermediate N

### (S)-tert-butyl 5-(4-hydroxyphenyl)pentane-1,2-diyldicarbamate

This compound is prepared analogously to Intermediate M, (R)-tert-butyl 5-(4-hydroxyphenyl)pentane-1,2-diyldicarbamate replacing Intermediate K, (4-((R)-4,5-Diamino-pentyl)-phenol with Intermediate L, 4-((S)-4,5-Diamino-pentyl)-phenol; [M+H]+395.

### Intermediate O

### (R)-3-[4-((R)-4,5-Diamino-pentyl)-phenoxy]-propane-1,2-diol

Step 1

Triethylamine (8.37 μl, 0.06 mmol) and (R)-(+)-glycidol (96 μl, 1.442 mmol) are added to a solution of (R)-tert-butyl 5-(4-hydroxyphenyl)pentane-1,2-diyldicarbamate (Intermediate M) (474 mg, 1.20 mmol) in EtOH (5 ml) and the resulting solution is heated at 90° C. for 18 hours. The reaction is allowed to cool to room temperature and concentrated in vacuo. Purification by chromatography (SiO2, EtOAc/iso-hexane) affords {(R)-2-tert-Butoxycarbonylamino-5-[4-((R)-2,3-dihydroxy-propoxy)-phenyl]-pentyl}-carbamic acid tert-butyl ester; [M+H]+469.

Step 2

{(R)-2-tert-Butoxycarbonylamino-5-[4-((R)-2,3-dihydroxy-propoxy)-phenyl]-pentyl}-carbamic acid tert-butyl ester (94 mg, 0.201 mmol) is stirred with a solution of 1 M HCl in Et2O (3 ml) for 18 hours and then loaded onto a 1 g SCX-2 cartridge washed with MeOH (30 ml), followed by 7M NH3 in MeOH (30 ml). The NH3 fraction is concentrated in vacuo to give the title compound, (R)-3-[4-((R)-4,5-Diamino-pentyl)-phenoxy]-propane-1,2-diol Intermediate H(R)-3-[4-((R)-4,5-Diamino-pentyl)-phenoxy]propane-1,2-diol; [M+H]+269.

### Intermediate P

### (R)-3-[4-((S)-4,5-Diamino-pentyl)-phenoxy]-propane-1,2-diol

This compound is prepared analogously to Intermediate O replacing (R)-tert-butyl 5-(4-hydroxyphenyl)pentane-1,2-diyldicarbamate (Intermediate M with (S)-tert-butyl 5-(4-hydroxyphenyl)pentane-1,2-diyldicarbamate (Intermediate N); [M+H]+269.

### Intermediate Q

### 2-[4-((R)-4,5-Diamino-pentyl)-phenoxy]-1-morpholin-4-yl-ethanone

(R)-tert-butyl 5-(4-hydroxyphenyl)pentane-1,2-diyldicarbamate (Intermediate M) (446 mg, 0.565 mmol) is dissolved in DMF (10 ml) and Cs2CO3 (368 mg, 1.131 mmol) and 2-bromo-1-morpholinethanone (118 mg, 0.565 mmol) are added. The reaction is stirred at room temperature for 40 minutes, then diluted with water (20 ml) and extracted with EtOAc (2×50 ml). The organic layers are dried over MgSO4 and the solvent concentrated in vacuo to give a clear oil. Purification by chromatography on a Waters 3000 prep HPLC system (Microsorb™ C18 Water/MeCN+0.1% TFA) yields a clear oil, which is dissolved in dioxane (4 ml) and treated with 4 M HCl in dioxane (4 ml) and stirred at room temperature for 4 days. Concentration in vacuo affords a white foam which is dissolved in MeOH (3 ml) and loaded onto a 10 g SCX-2 cartridge which is washed with MeOH (60 ml) and 7M NH3 in MeOH (60 ml). The NH3 fractions are combined and concentrated in vacuo to give the title compound as a colorless oil; [M+H]+322.

### Intermediate R

### 5-(4-Methoxy-phenyl)-hexane-1,2-diamine

This compound is prepared analogously to Intermediate I by replacing 4-(4-methoxyphenyl)-1-butanol with 4-(4-methoxyphenyl)-1-pentanol.

### Intermediate S

### ((S)-4,5-Diamino-pentyl)-carbamic acid benzyl ester

Step 1

Concentrated HCl (15 ml) is added to a suspension of Na-BOC—N8-Z-L-ornithine (5.00 g, 13.65 mmol) in 2,2-dimethoxypropane (150 ml). An endotherm occurs and the resulting solution is left to stir at room temperature for 6 hours. The solvent is then reduced in vacuo to approximately 50 ml and diethyl ether (100 ml) is added to turn the solution turbid. On stirring a thick white suspension forms. The white solid is collected by filtration and rinsed with diethyl ether (100 ml). The white solid is dissolved in MeOH (30 ml) and diethyl ether (200 ml) is added to precipitate a white solid that is collected by filtration and rinsed with diethyl ether. The solid is dissolved in DCM and washed with 2 N NaOH (75 ml). The organic phase is dried over MgSO4 and the solvent evaporated in vacuo to yield (S)-2-Amino-5-benzyloxycarbonylaminopentanoic acid methyl ester as a colorless oil; [M+H]+280.78.

Step 2

(S)-2-Amino-5-benzyloxycarbonylamino-pentanoic acid methyl ester (2.80 g, 9.99 mmol) and 7M NH3 in MeOH (20 ml) is stirred at room temperature for 72 hours. The reaction mixture is evaporated to dryness in vacuo to yield a white solid. The white solid is suspended in diethyl ether before filtration and drying to yield ((S)-4-Amino-4-carbamoyl-butyl)-carbamic acid benzyl ester.

Step 3

((S)-4-Amino-4-carbamoyl-butyl)-carbamic acid benzyl ester (1.87 g, 7.071 mmol) is suspended in dry THF (40 ml) and cooled to 10° C. in an ice bath under nitrogen. Borane (28.3 ml of a 1 M solution in THF, 28.3 mmol) is added. The ice bath is removed and the suspension heated to 70° C. and then left to stir at this temperature for 3 hours. Further borane (28.3 ml of a 1 M solution in THF, 28.3 mmol) is added and then after an hour the same amount of 1M borane in THF is added again. After a final hour at 70° C. the reaction mixture is quenched with MeOH (40 ml). The solvent is reduced in vacuo to approximately 50 ml. This is diluted with 5 M HCl (100 ml) and washed with diethyl ether (3×100 ml). The aqueous phase is basified to pH 12 with 2N NaOH and product extracted into EtOAc (3×100 ml). The organic phases are combined, dried over MgSO4 and the solvent evaporated in vacuo to yield the title compound as a colorless oil.

### Intermediate T

### 3,5-Diamino-6-chloro-pyrazine-2-carboxylic acid [(S)-4-(4-amino-butyl)-imidazolidin-(2E)-ylidene]-amide

To a suspension of (4-{(S)-2-[(E)-3,5-Diamino-6-chloro-pyrazine-2-carbonylimino]-imidazolidin-4-ybenzoyl}-butyl)-carbamic acid benzyl ester (Ex. 5) (0.110 g, 0.239 mmol) in dry DCM (20 ml) is added iodotrimethylsilane (0.130 ml, 0.956 mmol). The reaction mixture is stirred at room temperature for 3.5 hours. MeOH is added to the suspension yielding a solution. Purification by catch and release resin (SCX-2) eluting with MeOH and 7 M NH3 in MeOH yields the title compound as a brown oil; [M+H]+327.1

### Intermediate U

### 4-Amino-4-aminomethyl-piperidine-1-carboxylic acid tert-butyl ester

### Step 1

To a solution of 4-amino-4-cyano-piperidine-1-carboxylic acid tert-butyl ester (11.5 g, 51.0 mmol) in pyridine (20 ml) at 0° C. is added trifluoroacetic anhydride (11.0 ml) slowly and the reaction mixture is stirred at 0° C. for 4 h. The reaction mixture is diluted with DCM, washed with brine, dried over Na2SO4 and concentrated in vacuo. The residue obtained is dissolved in DCM and re-precipitated by adding petroleum ether. The supernatant solvent mixture is decanted and the product is washed again with petroleum ether and dried under vacuum to afford 4-Cyano-4-(2,2,2-trifluoro-acetylamino)-piperidine-1-carboxylic acid tert-butyl ester as an oil; 1H NMR (d6-DMSO): 1.40 (9H, s), 1.81-1.88 (2H, m), 2.26-2.32 (2H, m), 2.99-3.15 (2H, m), 3.79-3.82 (2H, m), 10.1 (1H, s).

### Step 2

To a solution of cyano-4-(2,2,2-trifluoro-acetylamino)-piperidine-1-carboxylic acid tert-butyl ester (10.0 g, 31.0 mmol) in EtOH (150 ml) is added Raney nickel (˜1.5 g) and the reaction mixture is stirred under an atmosphere of hydrogen for 3 days. A further quantity of Raney nickel (˜1.5 g) is added and the reaction mixture is further stirred for 2 days. The reaction mixture is filtered through a plug of Celite™ (filter material) and the filtrate is concentrated in vacuo to obtain 4-Aminomethyl-4-(2,2,2-trifluoro-acetylamino)-piperidine-1-carboxylic acid tert-butyl ester as a viscous oil that is used crude without further purification.

### Step 3

### 4-Amino-4-aminomethyl-piperidine-1-carboxylic acid tert-butyl ester

To a solution of 4-aminomethyl-4-(2,2,2-trifluoro-acetylamino)-piperidine-1-carboxylic acid tert-butyl ester in MeOH (70 ml) is added a 30% aqueous solution of ammonia (70 ml) and the reaction mixture is stirred at 80° C. overnight. The reaction mixture is concentrated in vacuo to 4-Amino-4-aminomethyl-piperidine-1-carboxylic acid tert-butyl ester as a brown oil that is used crude without further purification; [M+H]+230.

### Intermediate V

### 3-(3-Isopropoxy-propylsulfamoyl)-benzoic acid

3-Isopropoxypropylamine (1.1 eq.) is dissolved in THF with stirring at room temperature. N,N-diisopropylethylamine (1 eq.) is added followed by methyl 3-(chlorosulfonyl)benzoic acid (1 eq.). The reaction mixture is stirred at room temperature for 2 hours before the solvent is evaporated in vacuo to yield the crude titled product.

### Intermediate W

### 3-(3-Isopropyl-ureido)-benzoic acid

A suspension of 3-Aminobenzoic acid (20 g, 145.8 mmol) in THF (300 ml) is heated to 60° C. to form a clear solution. I-propylisocyanate (14.9 g, 175 mmol) is added over 30 minutes. During the addition the product starts to precipitate. After complete addition toluene (300 ml) is added. The reaction mixture is stirred at 60° C. for 4.5 hours. The heating bath is removed and the mixture is stirred overnight at room temperature. Finally the suspension is filtered and washed with a mixture of 1:1 THF: toluene (200 ml). The product is dried at 60° C. for 18 hours to yield 3-(3-Isopropyl-ureido)-benzoic acid.

### Intermediate X

### 5-Oxo-1-(3-pyrrol-1-yl-propyl)-pyrrolidine-3-carboxylic acid

Step 1

To a solution of 5-Oxo-pyrrolidine-3-carboxylic acid methyl ester (1 eq.) in dry DMF is added NaH (1.1 eq.) followed by 1-(3-bromo-propyl)-1H-pyrrole (1 eq.). The reaction mixture is stirred at room temperature overnight. Purification is by normal phase chromatography to yield 5-Oxo-1-(3-pyrrol-1-yl-propyl)-pyrrolidine-3-carboxylic acid methyl ester.

Step 2

To a cooled solution (0° C.) of 5-Oxo-1-(3-pyrrol-1-yl-propyl)-pyrrolidine-3-carboxylic acid methyl ester in THF, 0.2M LiOH is added and RM is stirred for 3 hours gradually warming to room temperature. Reaction mixture is acidified with 1N HCl and product extracted into ethyl acetate. The organic phase is washed with brine, dried over magnesium sulphate and the solvent evaporated in vacuo to yield 5-Oxo-1-(3-pyrrol-1-yl-propyl)-pyrrolidine-3-carboxylic acid.

### Intermediate Y

### 2-(3-Isopropyl-ureido)-isonicotinic acid

Step 1

To a solution of ethyl 2-aminoisonicotinate (500 mg, 3.01 mmol) in DMF (10 ml) is added triethylamine (1.26 ml, 9.03 mmol) and then isopropyl isocyanate (512 mg, 6.02 mmol). The reaction mixture is heated in a microwave at 140° C. for 2 hours. The reaction mixture is diluted with EtOAc, washed with water (×5), brine, dried (MgSO4) and concentrated in vacuo. Chromatography (SiO2, MeOH/DCM) affords 2-(3-Isopropyl-ureido)-isonicotinic acid ethyl ester; [M+H]+252.

Step 2

To a solution of 2-(3-Isopropyl-ureido)-isonicotinic acid ethyl ester (130 mg, 0.52 mmol) in MeOH (5 ml) is added 2 M NaOH (2.5 ml) and the resulting solution is stirred for 1.5 hours at room temperature. The solvent is removed in vacuo and sat. aq. NH4Cl solution is added. The pH of the aqueous phase is adjusted to 1 using 1 M HCl and the product extracted into EtOAc, dried (MgSO4) the solvent removed in vacuo to afford 2-(3-Isopropyl-ureido)-isonicotinic acid as a white solid; [M+H]+ 224.

### Intermediate Z

### 1-Isopropylcarbamoyl-1H-indole-4-carboxylic acid

This compound is prepared analogously to Intermediate Y by replacing ethyl 2-aminoisonicotinate in step 1 with methyl indol-4-carboxylate; [M+H]+247.

### Intermediate AA

### -4-(3-Isopropyl-ureido)-benzoic acid

This compound is prepared analogously to Intermediate Y by replacing ethyl 2-aminoisonicotinate in step 1 with methyl 4-aminobenzoate; [M+H]+237.

### Intermediate AB

### -6-(3-Isopropyl-ureido)-nicotinic acid

This compound is prepared analogously to Intermediate Y by replacing ethyl 2-aminoisonicotinate in step 1 with methyl 6-aminonicotinate; [M+H]+224.

### Intermediate AC

-[4-(2-Methoxy-ethoxymethoxy)-phenyl]-acetic acid

Step 1

To a solution of methyl 4-hydroxyphenylacetate (200 mg, 1.20 mmol) in DCM (5 ml) is added DIPEA (0.315 ml, 1.81 mmol), and then MEMCl (0.204 ml, 1.81 mmol), and the resulting reaction mixture is stirred for 2 hours at room temperature. An additional portion of MEMCl (0.102 ml, 1 mmol) and of DIPEA (0.158 ml, 1 mmol) are added, and the reaction mixture is stirred for a further 16 hours. An additional portion of MEMCl (0.102 ml, 1 mmol) and of DIPEA (0.158 ml, 1 mmol) are added and the reaction mixture is stirred for 3 hours. The reaction mixture is diluted with DCM and washed with 0.5 M HCl, 1 M NaOH and then 0.5 M HCl, dried (MgSO4) and concentrated in vacuo to afford [4-(2-Methoxy-ethoxymethoxy)-phenyl]-acetic acid methyl ester.

Step 2

To a solution of [4-(2-Methoxy-ethoxymethoxy)-phenyl]acetic acid methyl ester (192 mg, 0.76 mmol) in MeOH (3 ml) is added 2 M NaOH (3 ml). The reaction mixture is stirred for 16 hours at room temperature. The solvent is removed in vacuo and the residue dissolved in EtOAc and washed with. NH4Cl solution, dried (MgSO₄) and concentrated in vacuo to yield [4-(2-Methoxy-ethoxymethoxy)-phenyl]-acetic acid.

### Intermediate AD

### 3-[4-(2-Methoxy-ethoxymethoxy)-phenyl]-propionic acid

This compound is prepared analogously to Intermediate AC by replacing methyl 4-hydroxyphenylacetate in step 1 with methyl-3-(4-hydroxyphenyl)propionate.

### Intermediate AE

### 3-{4[2-(Tetrahydro-pyran-2-yloxy)-ethoxy]-phenyl}-propionic acid

Step 1

Methyl 3-(4-hydroxyphenyl)propianoate (0.1 g, 0.55 mmol) is dissolved in DMF (5 ml) and NaH (0.033 g of a 60% dispersion in mineral oil, 0.83 mmol) is added. The reaction mixture is stirred at room temperature for 15 minutes then 2-(2-bromethoxy)tehtrahydro-2-H-pyran (0.109 ml, 0.72 mmol) is added and the reaction mixture is left to stir for 18 hours. Dilution with EtOAc (50 ml), washing with water (25 ml), saturated NaHCO3 (25 ml) and brine (25 ml), drying over MgSO4, and concentration in vacuo yields 3-{442-(Tetrahydro-pyran-2-yloxy)-ethoxy]-phenyllpropionic acid methyl ester as a colorless oil; [M+H]+309.

Step 2

3-{4-[2-(Tetrahydro-pyran-2-yloxy)-ethoxy]-phenyl}-propionic acid methyl ester (0.12 g, 0.39 mmol) is dissolved in MeOH (3 ml) and 2M NaOH solution (3 ml) is added and the resulting solution is stirred at room temperature for 18 hours. The reaction mixture is diluted with saturated ammonium chloride solution (20 ml) and extracted with EtOAc (100 ml×2). The organic phased are combined, dried over MgSO4, the solvent removed in vacuo to yield the title compound as a colorless oil; [M+H]+=295.

### Intermediate AF

### 3-[4-(Pyridin-4-ylmethoxy)-phenyl]-propionic acid

Step 1

To a solution of Methyl 3-(4-hydroxyphenyl)propanoate (0.5 g, 2.77 mmol) in dry DMF (10 ml) is added potassium carbonate (0.76 g, 5.55 mmol) followed by 4-(bromomethyl)pyridine hydrobromide (0.7 g, 2.77 mmol). The reaction mixture is stirred at room temperature overnight then poured into water (80 ml) and extracted with EtOAc (40 ml). The organic phase is washed with brine, dried (MgSO4) and the solvent removed in vacuo to yield a dark brown oil. Chromatography (SiO2, EtOAc) yields 3-[4-(Pyridin-4-ylmethoxy)-phenyl]-propionic acid methyl ester as a colorless oil; [M+H]+272.0.

Step 2

To a solution of 3-[4-(Pyridin-4-ylmethoxy)-phenyl]-propionic acid methyl ester (0.28 g, 1.03 mmol) in THF (5 ml) and MeOH (5 ml) at room temperature is added 2 N LiOH (0.52 ml, 1.032 mmol) and the resulting solution is stirred overnight. Further 2 N LiOH (0.103 ml) is added and the reaction mixture stirred for a further 1 hour. The reaction mixture is concentrated in vacuo and the residue is diluted with water (50 ml) followed by EtOAc. The aqueous phase is acidified to pH 2 with 1N HCl, and extracted with DCM. The organic phase is concentrated to a third of its volume in vacuo until a white powder precipitates which is collected by filtration to yield the title compound; [M+H]+258.0.

### Intermediate AG

### 3-(4-tert-Butoxycarbonylmethoxy-phenyl)-propionic acid

Step 1

To a stirring solution of methyl 3-(4-hydroxyphenyl)propanoate (2 g, 11.10 mmol) in dry DMF (30 ml) at room temperature is added potassium carbonate (1.53 g, 10 mmol) followed by tert-butyl 2-bromoacetate (2.17 g, 11.10 mmol). The reaction mixture is purged with nitrogen, then stoppered and left stirring at room temperature for 7 days. The reaction mixture is poured into water (200 ml) and extracted with EtOAc (100 ml), washed with brine, dried (MgSO₄), filtered and evaporated in vacuo to yield a pale yellow oil. Flash chromatography (SiO2, EtOAc/iso-hexane) yields 3-(4-tert-Butoxycarbonylmethoxy-phenyl)-propionic acid methyl ester as a clear oil.

Step 2

To a solution of 3-(4-tert-Butoxycarbonylmethoxy-phenyl)-propionic acid methyl ester (2.70 g, 9.17 mmol) in THF (80 ml) is added 0.2N lithium hydroxide (45.9 ml, 9.17 mmol) at 0° C. and the reaction mixture is stirred at 0° C. for 4.5 hours. 1M HCl (15 ml) is added and the product is extracted using EtOAc (×3). The organic phase is dried (Na2SO4) and concentrated in vacuo to yield a white solid. Flash chromatography (SiO2, 10% EtOAc in CH2Cl2, then 20% EtOAc in CH2Cl2) yields 3-(4-tert-Butoxycarbonylmethoxy-phenyl)-propionic acid as a white solid.

### Intermediate AH

### 3-(4-Carbamoylmethoxy-phenyl)-propionic acid

This compound is prepared analogously to Intermediate AG by replacing tert-butyl 2-bromoacetate in step 1 with 2-bromoacetamide; [M+H]+530.1.

### Intermediate AI

### 1-[4-(2-Carboxy-ethyl)phenoxy]-cyclobutanecarboxylic acid ethyl ester

This compound is prepared analogously to Intermediate AG by replacing tert-butyl 2-bromoacetate in step 1 with ethyl 1-bromocyclobutane-carboxylate; [M+H]+293.0.

### Intermediate AJ

### 2-[4-(2-Carboxy-ethyl)-phenoxy]-2-methyl-propionic acid tert-butyl ester

This compound is prepared analogously to Intermediate AG by replacing tert-butyl 2-bromoacetate in step 1 with tert-butyl 2-bromoisobutyrate. 1H NMR (DMSO-d6): 1.40 (9H, s), 1.48 (6H, s), 2.49 (2H, t, J=7.5), 2.75 (2H, t, J=7.5), 6.71 (2H, d, J=8.5), 7.11 (2H, d, J=8.50), 12.10 (1H, s).

### Intermediate AK

### 3-(4-Methoxycarbonylmethoxy-phenyl)-propionic acid

Step 1

To a solution of 3-(4-hydroxyphenyl)propanoic acid (3.32 g, 20 mmol) in dry DMF (20 ml) is carefully added 1,1′-carbonyldiimidazole (3.24 g, 20 mmol) portionwise. The reaction mixture is stirred at 40° C. for 2 hours after which time DBU (6.02 ml, 40 mmol) and tert-butanol (4.78 ml, 50 mmol) are added and the reaction mixture is now stirred at 65° C. for 2 days. The reaction mixture is allowed to cool to room temperature and poured into water (50 ml) and the product is extracted with diethyl ether (3×30 ml). The organics are combined, dried (MgSO₄) and the solvent removed in vacuo to give a yellow oil. Purification by flash chromatography (SiO2, EtOAc/iso-hexane) yields 3-(4-Hydroxy-phenyl)-propionic acid tert-butyl ester as a colorless oil. ¹H NMR (DMSO-d6) 9.1 (1H, s), 7.0 (2H, d, J=8.45), 6.65 (2H, d, J=8.45), 2.7 (2H, t, J=7.28), 2.4 (2H, t, J=7.28), 1.4 (9H, s).

Step 2

To a solution of -(4-Hydroxy-phenyl)-propionic acid tert-butyl ester (1 g, 4.50 mmol) in dry DMF (20 ml) at room temperature under argon is added potassium carbonate (0.62 g, 4.50 mmol). followed by methyl bromoacetate (0.43 ml, 4.50 mmol) and the reaction mixture is stirred at room temperature. The reaction mixture is diluted with EtOAc and washed with water, dried (MgSO₄) and evaporated in vacuo to yield a clear colorless liquid. Purification on a Waters 3000 prep HPLC system (C18, MeCN/water) yields 3-(4-Methoxycarbonylmethoxy-phenyl)-propionic acid tert-butyl ester as a pale yellow oil.

Step 3

To 3-(4-Methoxycarbonylmethoxy-phenyl)-propionic acid tert-butyl ester (0.097 g, 0.33 mmol) is added a 90% solution of TFA in DCM (2 ml) and the resulting solution is stirred at room temperature for 1 hour. The solvents are removed in vacuo to yield 3-(4-Methoxycarbonylmethoxy-phenyl)-propionic acid as an off-white powder; [M+H−18]+256.0

### Intermediate AL

### 3-[4-(2-Propoxycarbonyl-ethyl)-phenyl]-propionic acid

To a solution of 3,3′-(1,4-phenylene)dipropanoic acid (250 mg, 1.125 mmol) DCM (15 ml) is added 4-dimethylaminopyridine (137 mg, 1.125 mmol) and propanol (3 ml, 40.1 mmol). The solution is cooled to 0° C. and dicyclohexylcarbodiimide (232 mg, 1.125 mmol) is added and the resulting solution is stirred at 0° C. for 30 minutes and 2 hours at room temperature. Concentration in vacuo affords a white solid which is suspended in Et2O (50 ml) and filtered to remove any insoluble material. The filtrate is concentrated in vacuo and purification by chromatography (SiO2, EtOAc/iso-hexane) affords the title compound.

### Intermediate AM

### 3-[4-(2-Ethoxycarbonyl-ethyl)-phenyl]-propionic acid

This compound is prepared analogously to Intermediate AL replacing propanol with ethanol.

### Intermediate AN

### 3-[4-(2-Methoxycarbonyl-ethyl)-phenyl]-propionic acid

This compound is prepared analogously to Intermediate AL replacing propanol with methanol.

### Intermediate AO

### 1-(2-Phenoxy-ethyl)-1H-indole-4-carboxylic acid

Step 1

NaH (60% dispersion in mineral oil, 68.5 mg, 1.71 mmol) is added to solution of methyl indole-4-carboxylate (200 mg, 1.142 mmol) in DMF (5 ml) and the resulting suspension is stirred at room temperature for 20 minutes. After this time (2-bromoethoxy)benzene (298 mg, 1.484 mmol) is added and the reaction is stirred at room temperature for 18 hours. Dilution with EtOAc (50 ml) and washing with water (25 ml×2), saturated NaHCO3 (25 ml) and brine (25 ml), drying over MgSO4, concentration in vacuo and purification by chromatography (SiO2, EtOAc/iso-hexane) affords 1-(2-Phenoxy-ethyl)-1H-indole-4-carboxylic acid methyl ester; [M+H]+296.

Step 2

1-(2-Phenoxy-ethyl)-1H-indole-4-carboxylic acid methyl ester (185 mg, 0.626 mmol) is suspended in a mixture of MeOH (3 ml) and 2 M NaOH (2 ml). The suspension is stirred at room temperature for 2 hours, THF (1 ml) is added and the reaction is heated at 60° C. for 1 hour. The reaction is allowed to cool to room temperature and diluted with sat. NH4Cl solution (10 ml), extracted with EtOAc (10 ml×3), dried over MgSO4, and concentrated in vacuo to give the title compound; [M+H]+282.

### Intermediate AP

### 1-(2-p-Tolyl-ethyl)-1H-indole-4-carboxylic acid

This compound is prepared analogously to Intermediate AO replacing (2-bromoethoxy)benzene with 4-methylphenethyl bromide; [M+H]+280.

### Intermediate AQ

### 1-[2-(Tetrahydro-pyran-2-yloxy)-ethyl]-1H-indole-4-carboxylic acid

This compound is prepared analogously to Intermediate AO replacing (2-bromoethoxy)benzene with 2-(2-bromoethoxy)tetrahydro-2H-pyran; [M+H]+290.

### Intermediate AR

### 1-[2-(4-Methoxy-phenoxy)-ethyl]-1H-indole-4-carboxylic acid

This compound is prepared analogously to Intermediate AO replacing (2-bromoethoxy)benzene with 1-(2-bromoethoxy)-4-methoxybenzene; [M+H]+312.

### Intermediate AS

### 1[2-(4-tert-Butyl-phenoxy)-ethyl]-1H-indole-4-carboxylic acid

This compound is prepared analogously to Intermediate AO replacing (2-bromoethoxy)benzene with 1-(2-bromoethoxy)-4-tert-butylbenzene; [M+H]+338.

### Intermediate AT

### 1-(2-[1,3]Dioxan-2-yl-ethyl)-1H-indole-4-carboxylic acid

This compound is prepared analogously to Intermediate AO replacing (2-bromoethoxy)benzene with (2-bromethyl)1,3-dioxane; [M+H]+276.

### Intermediate AU

### 2,3-Dimethyl-[2-(tetrahydro-pyran-2-yloxy)-ethyl]-1H-indole-5-carboxylic acid

This compound is prepared analogously to Intermediate A replacing (2-bromoethoxy)benzene with (2-(2-bromoethoxy)tetrahydro-2H-pyran and replacing Methyl indole-4-carboxylate with 2,3-dimethyl-1H-indole-5-carboxylate; [M+H]+318.

### Intermediate AV

### 1-(4,4,4-Trimethoxy-butyl)-1H-indole-4-carboxylic acid

This compound is prepared analogously to Intermediate AO 1-(2-Phenoxy-ethyl)-1H-indole-4-carboxylic acid replacing (2-bromoethoxy)benzene with trimethyl 4-bromoorthobutyrate.

### Intermediate AW

### 1-[2-(2-Methoxy-ethoxymethoxy)-ethyl]-1H-indole-4-carboxylic acid

Step 1

NaH (60% dispersion in mineral oil, 86 mg, 2.14 mmol) is added to a solution of methyl indole-4-carboxylate (250 mg, 1.427 mmol) in DMF (20 ml) and the resulting suspension is stirred at room temperature for 30 minutes. After this time (2-(2-bromoethoxy)tetrahydro-2H-pyran (388 mg, 1.86 mmol) is added and the reaction is stirred at room temperature for 22 hours. Dilution with EtOAc (50 ml), washing with water (25 ml×3), saturated NaHCO3 (25 ml×2) and brine (25 ml), drying over MgSO4, concentration in vacuo and purification by chromatography (SiO2, DCM/MeOH) affords 1[2-(Tetrahydro-pyran-2-yloxy)-ethyl]-1H-indole-4-carboxylic acid methyl ester; [M+H]+304.

Step 2

To a solution of 1[2-(Tetrahydro-pyran-2-yloxy)-ethyl]-1H-indole-4-carboxylic acid methyl ester (120 mg, 0.396 mmol) in MeOH (10 ml) is added p-toluenesulfonic acid monohydrate (7.25 mg, 0.04 mmol). The reaction is stirred at room temperature for 16 hours and the solvent is removed in vacuo. The residue is dissolved in MeOH (3 ml) and loaded onto a 1 g PEAX cartridge washed with MeOH (20 ml). The filtrate is concentrated in vacuo to give 1-(2-Hydroxy-ethyl)-1H-indole-4-carboxylic acid methyl ester; [M+H]+220.

Step 3

To a solution of -(2-Hydroxy-ethyl)-1H-indole-4-carboxylic acid methyl ester in DCM (3 ml) is added DIPEA (0.129 ml, 0.739 mmol) and 1-Chloromethoxy-2-methoxy-ethane (0.084 ml, 0.739 mmol). The solution is stirred at room temperature for 72 hours. The reaction is diluted with DCM (50 ml) and washed with 0.5 M HCl (20 ml), 1 M NaOH (20 ml) and 0.5 M HCl (20 ml). The organic layer is dried over MgSO4 and the solvent is removed in vacuo. Purification by chromatography (SiO2, DCM/MeOH) affords 1-[2-(2-Methoxy-ethoxymethoxy)-ethyl]-1H-indole-4-carboxylic acid methyl ester; [M+H]+308.

Step 4

To a solution of 1-[2-(2-Methoxy-ethoxymethoxy)-ethyl]-1H-indole-4-carboxylic acid methyl ester (69 mg, 0.225 mmol) in MeOH (2 ml) is added 2 M NaOH (1 ml) and the reaction is stirred at room temperature for 19.5 hours, then for 2 hours at 50° C. The reaction is allowed to cool to room temperature and the solvent removed in vacuo. To the residue is added sat. NH4Cl (10 ml), and the product is extracted with EtOAc (5×25 ml), washed with brine (10 ml), dried over Na2SO4, and the solvent is removed in vacuo, to give the title compound 1-[2-(2-Methoxy-ethoxymethoxy)-ethyl]-1H-indole-4-carboxylic acid; [M+H]+294.

### Intermediate AX

### 1-Diethylcarbamoylmethyl-1H-indole-4-carboxylic acid

Step 1

Methyl indole-4-carboxylate (50 mg, 2.85 mmol) and 2-chloro-N,N-diethylacetamide (854 mg, 5.71 mmol) are dissolved in DMF (10 ml) and to the solution is added potassium carbonate (986 mg, 7.14 mmol). The reaction is heated using microwave radiation at 100° C. for 2 hours, then diluted with DCM (60 ml) and washed with water (5×10 ml). Drying over MgSO4, concentration in vacuo, and trituration with Et20 affords 1-Diethylcarbamoylmethyl-1H-indole-4-carboxylic acid methyl ester; [M+H]+289.

Step 2

To a solution of Diethylcarbamoylmethyl-1H-indole-4-carboxylic acid methyl ester (480 mg, 1.665 mmol) in MeOH (5 ml) is added 2 M NaOH (5 ml). The reaction is heated at 50° C. for 20 hours and then allowed to cool to room temperature. The solvent is removed in vacuo and the residue dissolved in water (10 ml). The pH of the solution is adjusted to 5 using 1 M HCl and the resulting solid is collected by filtration to give the title compound 1-Diethylcarbamoylmethyl-1H-indole-4-carboxylic acid; [M+H]+275.

### Intermediate AY

### 4-[6-((R)-2,2-Dimethyl-[1,3]dioxolan-4-ylmethoxy)-naphthalen-2-ylmethoxy]-benzoic acid

Step 1

To a solution of methyl 6-hydroxy-2-naphthoate (4.55 g, 22.5 mmol) in anhydrous acetone (60 ml) are added S-(−)-glycidol (2.0 g, 27.0 mmol) and K2CO3 (9.3 g, 67.3 mmol). The reaction mixture is heated to reflux for 3 days. The reaction mixture is filtered through Celite™ (filter material) and the filtrate is concentrated in vacuo to afford 6-((S)-2,3-Dihydroxy-propoxy)-naphthalene-2-carboxylic acid methyl ester as a white solid; 1H NMR (DMSO-d6): 3.49 (2H, t, J=6.0 Hz), 3.85-3.88 (1H, m), 3.89 (3H, s), 4.02 (1H, dd, J=9.9, 6.0 Hz), 4.16 (1H, dd, J=9.9, 4.0 Hz), 4.73 (1H, t, J=6.0 Hz), 5.04 (1H, d, J=5.2 Hz), 7.26 (1H, dd, J=9.0, 2.0 Hz), 7.41 (1H, d, J=2.0 Hz), 7.88-7.94 (2H, m), 8.04 (1H, d, J=9.0 Hz), 8.55 (1H, s).

Step 2

To 6-((S)-2,3-dihydroxy-propoxy)-naphthalene-2-carboxylic acid methyl ester (0.9 g, 3.26 mmol) in anhydrous DMF (10 ml) is added 2,2-dimethoxypropane (2.0 ml, 16.3 mmol) and pyridinium p-toluenesulfonate (0.08 g, 0.32 mmol) and the reaction mixture is stirred at room temperature for 16 hours. The reaction mixture is concentrated in vacuo and the residue is dissolved in EtOAc. The EtOAc layer is washed with 10% NaHCO3, water, and brine, dried over anhydrous Na₂SO4 and the solvent is evaporated in vacuo to obtain 6-((R)-2,2-Dimethyl-[1,3]dioxolan-4-ylmethoxy)-naphthalene-2-carboxylic acid methyl ester as solid; 1H NMR (DMSO-d6): 1.32 (3H, s), 1.37 (3H, s), 3.78-3.82 (1H, m), 3.88 (3H, s), 4.benzoyl}-4.20 (3H, m), 4.45-4.50 (1H, m), 7.26 (1H, dd, J=9.0, 2.0 Hz), 7.45 (1H, d, J=2.0 Hz), 7.88 (1H, d, J=9.0 Hz), 7.93 (1H, d, J=9.0 Hz), 8.04 (1H, d, J=9.0 Hz), 8.55 (1H, s).

Step 3

To a solution of 6-((R)-2,2-dimethyl-[1,3]dioxolan-4-ylmethoxy)-naphthalene-2-carboxylic acid methyl ester (1.0 g, 3.16 mmol) in anhydrous THF (20 ml) at 0° C. is added LiAlH4 (1.9 ml of a 2M solution in THF, 3.8 mmol). The reaction mixture is stirred at room temperature overnight. The reaction mixture is concentrated in vacuo and the residue is purified by column chromatography (SiO2, DCM) to afford [6-((R)-2,2-Dimethyl-[1,3]dioxolan-4-ylmethoxy)-naphthalen-2-yl]-MeOH as a colorless viscous oil which solidified on standing; 1H NMR (d6-DMSO): 1.32 (3H, s), 1.37 (3H, s), 3.78 (1H, dd, J=8.3, 6.0 Hz), 4.01-4.15 (3H, m), 4.45-4.48 (1H, m), 4.60 (2H, d, J=6.0 Hz), 5.24 (1H, t, J=6.0 Hz), 7.14 (1H, dd, J=8.5, 2.5 Hz), 7.32 (1H, d, J=2.5 Hz), 7.41 (1H, dd, J=8.5, 1.5 Hz), 7.33-7.80 (3H, m).

Step 4

A mixture of methyl 4-hydroxybenzoate (0.5 g, 3.28 mmol), [6-((R)-2,2-dimethyl-[1,3]dioxolan-4-ylmethoxy)-naphthalen-2-yl]-methanol (0.9 g, 3.12 mmol) and triphenylphosphine (0.83 g, 3.16 mmol) in DCM (20 ml) is cooled to 0° C. Diethyl azodicarboxylate (0.5 ml, 3.17 mmol) is added dropwise. The reaction mixture is stirred at room temperature overnight. The reaction mixture is concentrated in vacuo and purified by column chromatography (SiO2, EtOAc/iso-hexane) to obtain white solid. The product obtained is once again purified by column chromatography (neutral alumina, EtOAc/petroleum ether) to obtain 4-[6-((R)-2,2-Dimethyl-[1,3]dioxolan-4-ylmethoxy)-naphthalen-2-ylmethoxy]-benzoic acid methyl ester as white solid; 1H NMR (d6-DMSO): 1.32 (3H, s), 1.38 (3H, s), 3.77-3.82 (4H, m), 4.08-4.16 (3H, m), 4.46-4.49 (1H, m), 5.30 (2H, s), 7.15-7.21 (3H, m), 7.37 (1H, d, J=2.0 Hz), 7.53 (1H, dd, J=8.50, 1.5 Hz), 7.83 (2H, dd, J=9.0, 6.0 Hz), 7.92 (3H, m).

Step 5

To a solution of 446-((R)-2,2-dimethyl-[1,3]dioxolan-4-ylmethoxy)-naphthalen-2-ylmethoxy]-benzoic acid methyl ester (0.46, 1.09 mmol) in THF/water (10 ml of a 1:1 mixture) is added lithium hydroxide (0.15 g, 3.57 mmol). The reaction mixture is stirred at room temperature overnight, then at 70° C. for 24 h. The reaction mixture is cooled to room temperature, neutralized with 1.5 M HCl and the white solid obtained is collected by vacuum filtration, washed with water and dried under vacuum to afford 4-[6-((R)-2,2-Dimethyl-[1,3]dioxolan-4-ylmethoxy)-naphthalen-2-ylmethoxy]-benzoic acid. [M]−407.

### Intermediate AZ

### 4-{3-[4-((R)-2,2-Dimethyl-[1,3]dioxolan-4-ylmethoxy)-phenyl]-propoxy}-benzoic acid

This compound is prepared analogously to Intermediate AY by replacing [6-((R)-2,2-dimethyl-[1,3]dioxolan-4-ylmethoxy)-naphthalen-2-yl]-methanol in Step 4 with 3-[4-((R)-2,2-dimethyl-[1,3]dioxolan-4-ylmethoxy)-phenyl]-propan-1-ol; 1H NMR (DMSO-d6): 1.30 (3H, s), 1.35 (3H, s), 1.97-2.01 (2H, m), 2.68 (2H, t, J=7.5 Hz), 3.72-3.75 (1H, m), 3.93-4.00 (4H, m), 4.06-4.10 (1H, m), 4.38 (1H, dd, J=6.0, 5.0), 6.87 (2H, d, J=9.0 Hz), 6.92 (2H, d, J=9.0 Hz), 7.14 (2H, d, J=9.0 Hz), 7.84 (2H, d, J=9.0 Hz).

### Intermediate BA

### 4-{2-[(E)-3,5-Diamino-6-chloro-pyrazine-2-carbonylimino]-1,3,8-triaza-spiro[4.5]decane-8-carbonyl}-piperidine-1-carboxylic acid tert-butyl ester

This compound is prepared analogously to Example 97 by replacing 4-benzyloxyphenylacetic acid with 1-Boc-piperidine-4-carboxylic acid; [M+H]+536.

### Intermediate BB

### 4-[(Naphthalene-1-sulfonylamino)-methyl]benzoic acid

4 N NaOH solution (30 ml) is added to a suspension of 4-(aminomethyl)benzoic acid (5.01 g, 31.82 mmol) in acetone (100 ml). Toluene (100 ml) is added and the reaction is heated at 40° C. to obtain dissolution. The solution is cooled to 0° C. and treated with 1-naphthalene sulfonyl chloride (12 g, 51.35 mmol) in acetone (100 ml) and the resulting reaction mixture is stirred for 3 hours. The reaction is acidified using citric acid and concentrated in vacuo. The residue is taken up in EtOAc and washed with water. The aqueous layer is back extracted with EtOAc and the combined organic layers are washed with water, brine, dried (Na2SO4) and the solvent removed in vacuo to yield a light brown solid. Trituration with Et2O yields the title compound.

### Intermediate BC

### 3-(Cyclohexyl-methyl-sulfamoyl)-4-methoxy-benzoic acid

Step 1

A solution of methyl 3-(chlorosulfonyl)-4-methoxybenzoate (2.0 g, 7.56 mmol) and diisopropylethylamine (1.94 ml, 11.34 mmol) in DCM (50 ml) is treated with N-methyl cyclohexylamine (0.70 ml, 9.07 mmol) at 0° C. The solution is stirred at room temperature for 3 hours and N-methyl cyclohexylamine (0.70 ml, 9.07 mmol) is added. The solution is partitioned between DCM (250 ml) and 0.5N HCl (100 ml). The organic layer is washed with 0.5N HCl (2×100 ml), NaHCO3 (2×100 ml) and water (100 ml), dried over MgSO4, and the solvent removed in vacuo to yield a yellow oil. Crystallisation (iPr2O/EtOAc) yields 3-(Cyclohexyl-methyl-sulfamoyl)-4-methoxy-benzoic acid methyl ester as yellow crystals; [M+H]+342.

Step 2

A solution of 3-(Cyclohexyl-methyl-sulfamoyl)-4-methoxy-benzoic acid methyl ester (1.50 g, 4.39 mmol) in 1,4 dioxane (40 ml) is treated with 2 N NaOH (10 ml) and the resulting solution is stirred at room temperature for 21 hours. The solvent is removed in vacuo and ice cold 2N HCl (25 ml) is added and the white solid which forms is extracted into DCM (150 ml). The organic layer is washed with water, dried (MgSO4) and the solvent removed in vacuo to yield the title compound as a white solid; [M−1]−326.

### Intermediate BD

### 3-Chloro-5-methoxy-4-[2-(4-methyl-piperazin-1-yl)-ethoxy]-benzoic acid

Step 1

A mixture of 5-chlorovanillic acid (5.0 g, 24.6 mmol) and conc. HCl (5 ml) in MeOH (100 ml) is heated at reflux for 48 hours. The solvent is removed in vacuo and water is added to the residue to yield a white precipitate, which is collected by filtration, washed with water, and then dissolved in Et2O. The solution is dried (Na2SO4) and the solvent removed in vacuo to yield 3-Chloro-4-hydroxy-5-methoxy-benzoic acid methyl ester as a white solid.

Step 2

Triphenylphosphine (6.4 g, 24.4 mmol) and DIAD (4.8 ml, 202.2 mmol) are added to a solution of 3-Chloro-4-hydroxy-5-methoxy-benzoic acid methyl ester (2.5 g, benzoyl} 0.5 mmol) in THF (40 ml) at 0° C. and the resulting solution is stirred for 2 hours at 0° C. and 16 hours at room temperature. The solvent is removed in vacuo, and water is added to the residue. The product is extracted in EtOAc, dried (Na2SO4) and the solvent removed in vacuo to afford a yellow oil. Flash chromatography (SiO2, EtOAc/MeOH) yields 3-Chloro-5-methoxy-4-[2-(4-methyl-piperazin-1-yl)-ethoxy]-benzoic acid methyl ester as an orange solid.

Step 3

A solution of 3-Chloro-5-methoxy-4-[2-(4-methyl-piperazin-1-yl)-ethoxy]-benzoic acid methyl ester (3.7 g, 10.7 mmol) in 2 N NaOH (20 ml) and THF (40 ml) is heated at reflux for 1 hour. The reaction mixture is washed with Et2O. The aqueous phase is concentrated in vacuo, and water (50 ml) is added. The pH is adjusted to 3-4 using 2N HCl. To this solution is added DOWEX 50WX4 (previously washed with MeOH, 2N HCl and water), and the resulting mixture is stirred at room temperature for 1 hour. The resin is filtered, washed with water, and the product is released from the resin by washing with MeOH/NH4OH. The solution is concentrated in vacuo, diluted with DCM and MeOH, dried (Na2SO4) and the solvent removed in vacuo to yield the title compound as a light cream solid.

### Intermediate BE

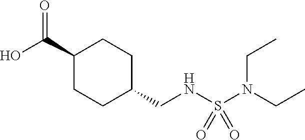 [SMILES: CCN(CC)S(=O)(=O)NC[C@H]1CC[C@H](C(=O)O)CC1]

Step 1

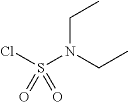

To a stirred solution of diethyl amine (500 ml, 4.8 mol) in Et₂O (1200 ml) is added sulfuryl chloride (177.3 ml, 2.19 mol) over 80 minutes at −15° C. The reaction is stirred at room temperature for 2.5 hours. Et₂O (1000 ml) is added and the white solid present is removed by filtration, and washed with Et₂O (2000 ml). The combined filtrates are concentrated under reduced pressure to yield as a colorless oil.

Step 2

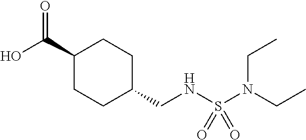 [SMILES: CCN(CC)S(=O)(=O)NC[C@H]1CC[C@H](C(=O)O)CC1]

To a stirred solution of trans-4-(aminomethyl)-cyclohexane carboxylic acid (10 g, 63.6 mmol) in 1N NaOH (153 ml) is added (10.91 g, 63.6 mmol) and the resulting mixture is stirred at room temperature for 15 hours. The reaction is cooled to 10° C. and conc. HCl solution (15 ml) is added and the mixture stirred for 10 minutes at this temperature. White crystals form which are isolated by filtration and washed with Et₂O (40 ml) to yield the title compound.

### Intermediate BF

### 3-(3-Phenyl-isoxazol-5-yl)-propionic acid

This compound is prepared as described by G. S. d'Alcontres; C Caristi; A Ferlazzo; M Gattuso, J. Chem. Soc. Perkin 1, (1976) 16, 1694.

### Intermediate BG

### 3-(4-Chloro-phenoxymethyl)-benzylamine

This compound is prepared as described in US 2008200523.

### Intermediate BH

### 2-{4-[2-(4-Fluoro-phenyl)-ethoxy]-phenyl}-ethylamine

### Step 1

A suspension of 4-Hydroxybenzyl cyanide (7.9 g, 59.57 mmol), 1-(2-Bromo-ethyl)-4-fluoro-benzene (17.4 g, 71.48 mmol), potassium carbonate (19.8 g, 143 mmol) and sodium iodide (2.68 g, 17.87 mmol) in acetonitrile (120 ml) is heated at reflux for 44 hours. The reaction mixture is cooled and filtered and the solvent removed in vacuo to yield a dark brown oil. Flash chromatography (SiO₂, EtOAc/iso-hexane) yields {442-(4-Fluoro-phenyl)-ethoxy}-phenyl}-acetonitrile as a yellow oil.

### Step 2

2 N NaOH solution (45.2 ml, 90.3 mmol) is added to a solution of {4-[2-(4-Fluoro-phenyl)-ethoxy]-phenyl}-acetonitrile (3.29 g, 12.9 mmol) in EtOH (45.2 mol) followed by Al—Ni Alloy (2.5 g) and the resulting reaction mixture is stirred for 1 hour at room temperature. The reaction mixture is filtered and the EtOH removed in vacuo. The product is extracted into DCM (2×80 ml), dried (MgSO₄) and the solvent removed in vacuo to yield the title compound as a yellow oil.

### Intermediate BI

### 2-(4,6-Dimethyl-1H-indol-3-yl)-ethylamine

This compound is prepared as described in EP 620222.

### Intermediate BJ

### 2-[4-(4-Phenyl-butoxy)-phenyl]-ethylamine

This compound is prepared as described in WOP 2004016601.

### Intermediate BK

### 4-(5-Methyl-2-phenyl-oxazol-4-ylmethoxy)-benzenesulfonyl chloride

This compound is prepared as described in WO 2005026134.

### Intermediate BL

### 2-Phenyl-3H-benzoimidazole-5-sulfonyl chloride

This compound is prepared as described in EP 1205475.

### Intermediate BM

### 4-Aminomethyl-1-(1-phenyl-ethyl)-piperidin-4-ylamine

### Step 1

1-(1-Phenyl-ethyl)-piperidin-4-one is prepared according to the procedure described on page 525 of J. Org. Chem. 1991, 56(2), 513-528.

To a mixture of 1-(1-phenyl-ethyl)-piperidin-4-one (10.9 g, 53.6 mmol), ammonium chloride (4.3 g, 80.4 mmol) and 30% aqueous ammonia solution (30 ml) in water (30 ml) at room temperature is added sodium cyanide (4.0 g, 81.6 mmol) portion wise. The reaction mixture is stirred at room temperature for 18 hours, then diluted with water and extracted with DCM. The organic phase is washed with brine, dried over Na₂SO₄, filtered and concentrated in vacuo to obtain 4-Amino-1-(1-phenyl-ethyl)-piperidine-4-carbonitrile as a brown oil; [M+H]⁺230.

### Step 2

4-Aminomethyl-1-(1-phenyl-ethyl)-piperidin-4-ylaminen is prepared analogously to Intermediate U by replacing 4-amino-4-cyano-piperidine-1-carboxylic acid tert-butyl ester in Step 1 with 4-amino-1-(1-phenyl-ethyl)-piperidine-4-carbonitrile; [M+H]⁺234.

### Intermediate BN

### 4-Aminomethyl-1-(4-methoxy-benzyl)-piperidin-4-ylamine

This compound is prepared analogously to Intermediate BM by replacing 1-(1-phenyl-ethyl)-piperidin-4-one with 1-(4-methoxybenzyl)piperidin-4-one in step 2; ¹H NMR (DMSO-d6): 1.46-1.64 (4H, m), 2.38-2.55 (4H, m), 2.67 (2H, s), 3.26 (2H, s), 4.08 (3H, s), 6.87 (2H, d, J=8.2 Hz), 7.18 (2H, d, J=8.2 Hz).

### Intermediate BO

### 4-Aminomethyl-1-pyridin-4-ylmethyl-piperidin-4-ylamine

### Step 1

To a solution of 4-aminomethyl-4-(2,2,2-trifluoro-acetylamino)-piperidine-1-carboxylic acid tert-butyl ester (Intermediate U, Step 2) (5.0 g, 15.4 mmol) in DCM (50 ml) at 0° C. is added pyridine (10 ml) followed by trifluoroacetic anhydride (3.5 ml, 25.3 mmol) and the reaction mixture is stirred at room temperature for 16 hours. The reaction mixture is diluted with DCM, washed with brine, dried over Na₂SO₄ and concentrated in vacuo. The residue obtained is dissolved in diethyl ether and re-precipitated by adding petroleum ether. The solvent mixture is decanted and the solid dried under vacuum to afford 4-(2,2,2-Trifluoro-acetylamino)-4-[(2,2,2-trifluoroacetylamino)-methyl]-piperidine-1-carboxylic acid tert-butyl ester; [M+H]⁺420.

### Step 2

To a solution of 4-(2,2,2-trifluoro-acetylamino)-4-[(2,2,2-trifluoro-acetylamino)-methyl]-piperidine-1-carboxylic acid tert-butyl ester (5.25 g, 12.5 mmol) in dioxane (50 ml) is added 4 M HCl in dioxane (15 ml) and the reaction mixture is stirred at room temperature for 3 hours. The reaction mixture is concentrated in vacuo and the off-white solid obtained dissolved in the minimum amount of MeOH and re-precipitated by adding diethyl ether. The supernatant solvent mixture is decanted and the product is washed again with diethyl ether and dried under vacuum to afford 2,2,2-Trifluoro-N-{4-[(2,2,2-trifluoro-acetylamino)-methyl]-piperidin-4-yl}-acetamide hydrochloride; [M+H]⁺322.

### Step 3

To a suspension of NaH (170 mg of a 60% dispersion in mineral oil, 4.25 mmol) in anhydrous DMF (20 ml) is added 2,2,2-trifluoro-N-{4-[(2,2,2-trifluoro-acetylamino)-methyl]-piperidin-4-yl}-acetamide hydrochloride) (500 mg, 1.4 mmol) followed by 4-bromomethylpyridine hydrobromide (350 mg, 1.4 mmol). The reaction mixture is stirred at room temperature for 3 hours. The reaction mixture is quenched with sat. NH₄Cl solution and is concentrated in vacuo. The residue is purified by column chromatography (basic alumina, MeOH/DCM) to obtain 2,2,2-Trifluoro-N-[1-pyridin-4-ylmethyl-4-(2,2,2-trifluoro-acetylamino)-piperidin-4-ylmethyl]-acetamide as off-white solid; [M+H]⁺413.

### Step 4

To a solution of 2,2,2-trifluoro-N-[1-pyridin-4-ylmethyl-4-(2,2,2-trifluoro-acetylamino)-piperidin-4-ylmethyl]-acetamide (200 mg, 0.49 mmol) in MeOH (10 ml) is added 30% aqueous ammonia solution (10 ml) and the reaction mixture is stirred at 60° C. for 3 h. The reaction mixture is concentrated in vacuo to obtain 4-Aminomethyl-1-pyridin-4-ylmethyl-piperidin-4-ylamine as a colorless gummy oil that is used without further purification; ¹H NMR (DMSO-d6): 1.63-1.77 (4H, m), 2.45-2.54 (4H, m), 2.49 (2H, s), 3.57 (3H, s), 7.30 (2H, d, J=5.5 Hz), 8.68 (2H, d, J=5.5 Hz).

### Intermediate BP

### 4-Aminomethyl-1-(3-phenyl-propyl)-piperidin-4-ylamine

This compound is prepared analogously to Intermediate BO by replacing -bromomethylpyridine hydrobromide (Step 3) with 1-bromo-3-phenylpropane; [M+H]⁺248.

### Intermediate BQ

### 4-Aminomethyl-1-cyclohexylmethyl-piperidin-4-ylamine

This compound is prepared analogously to Intermediate BO by replacing -bromomethylpyridine hydrobromide (Step 3) with cyclohexylmethylbromide. This intermediate is used crude in the preparation of Example 250.

### Intermediate BR

### 3-Amino-3-aminomethyl-8-aza-bicyclo[3.2.1]octane-8-carboxylic acid tert-butyl ester

This compound is prepared analogously to Intermediate BM by replacing 1-(1-phenyl-ethyl)-piperidin-4-one (Step 1) with N-Boc-nortropinone; ¹H NMR (DMSO-d6): 1.40 (9H, s), 1.63-1.85 (8H, m), 2.79 (2H, s), 4.06 (2H, s).

### IV. Formulations

In one aspect, the invention features a pharmaceutical formulation comprising an inhibitor of ENaC activity as provided in Column D and a modulator of CF Modulator activity as provided in Columns A, B, or C according to Table I. In some embodiments, the modulator of CF Modulator activity can include a compound of Formula I, or a compound of Formula II, or a compound of Formula III, or combinations thereof according to Table I. In some embodiments, the modulator of CF Modulator activity can include Compound 1, or Compound 2, or Compound 3 or combinations thereof according to Table I.

Table I is reproduced here for convenience.

TABLE I
Compounds

| Column A | | Column B | | Column C | | Column D | | Column E | |
|---|---|---|---|---|---|---|---|---|---|
| Embodiments | | Embodiments | | Embodiments | | Embodiments | | Embodiments | |
| Section | Heading | Section | Heading | Section | Heading | Section | Heading | Section | Heading |
| II.A.1. | Compound | II.B.1. | Compound | II.C.1. | Compound | II.D.1. | Compound | II.E.1. | ENAC |
| | of Formula | | of Formula | | of Formula | | of Formula | | Compounds |
| | A | | B | | C | | D | | |
| II.A.2 | Compound | II.B.2 | Compound | II.C.2 | Compound | II.D.2 | Compound | II.E.2 | Compound |
| | of Formula | | of Formula | | of Formula | | of Formula | | of Formula E |
| | A1 | | B1 & B2 | | C1 | | D1 | | |
| II.A.3. | Compound | | | II.C.3. | Compound | II.D.3. | Compound | | |
| | 1 | | | | 2 | | 3 | | |

Formulations Containing an ABC Transporter Modulator and an ENaC Inhibitor.

In various embodiments, the present invention also provides formulations comprising at least one component from Columns A, or B, or C, or D and at least one component from Column E for the treatment of a condition, disease, or disorder implicated by CFTR and/or ENaC dysfunction. The formulations can comprise any number of pharmaceutically acceptable dosage forms including, solid forms such as: tablets, mini-tablets, micro-tablets, particles, mini-particles, microparticles, powders, trouches, capsules, pellets, mini-pellets and the like commonly employed in oral administration of pharmaceuticals. These solid forms may be formulated using compressed or compacted powders, granules and other variably sized particles. In still other embodiments, the pharmaceutical compositions described herein may be formulated into liquid forms for parenteral or enteral administration. Illustrative dosage forms described above, can include pharmaceutically acceptable excipients and carriers which are generally known to those skilled in the art and are thus included in the instant invention. Such excipients and carriers are described, for example, in “Remingtons Pharmaceutical Sciences” Mack Pub. Co., New Jersey (1991), which is incorporated herein by reference.

The formulations of the invention may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into granules, mini-tablets, pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

The pharmaceutical composition of Table I can be administered in one vehicle or separately. In another aspect, the pharmaceutical combination composition comprising an inhibitor of ENaC activity as exemplified in Column D of Table I, can be formulated into a unitary dosage unit, for example, a tablet, a capsule, a liquid suspension or solution for administration to the mammal in need thereof. The ENaC inhibitor can include an amorphous form, a substantially amorphous form or a crystalline form of the ENaC compound. Alternatively, each active agent can be formulated separately as a single dosage unit to be administered with the other active agent of the combination concurrently, or sequentially, i.e. prior to, or subsequent to each other, or within predetermined time periods apart, for example, within 5 minutes, within 30 minutes, within 1 hr., within 2 hrs, within 3 hrs. within 6 hrs., or within 12 hrs from administration of the other active agent. In some embodiments, the time period may be 24 hrs or more. For example, the first active agent (ENaC inhibitor or CF Modulator modulator) is administered on day 1, and the second active agent of the combination is administered the next day. The sequential administration regime is intended to only exemplify one of a number of possibilities of delayed administration of the second active agent from the first active agent and could be readily determined by one of ordinary skill in the art, for example, a prescribing physician.

The pharmaceutical compositions described herein may encompass one active agent or two different active agents selected from Table I, with the understanding that if the formulation includes two active agents, one of the active agents is an inhibitor of ENaC activity as exemplified by the components of Column D and the other active agent is a modulator of CF Modulator activity exemplified by the components of Columns A-C. In some embodiments, the pharmaceutical composition may contain more than one CF Modulator modulator as provided in Columns A-C.

In some embodiments, the pharmaceutical composition optionally comprises a pharmaceutically acceptable carrier, adjuvant or vehicle. In certain embodiments, these compositions optionally further comprise one or more additional therapeutic agents.

It will also be appreciated that certain of the Compounds of present invention can exist in free form for treatment, or where appropriate, as a pharmaceutically acceptable derivative, enantiomer, tautomer or a prodrug thereof. According to the present invention, a pharmaceutically acceptable derivative or a prodrug includes, but is not limited to, pharmaceutically acceptable salts, esters, salts of such esters, or any other adduct or derivative which upon administration to a patient in need thereof is capable of providing, directly or indirectly, a Compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a Compound of this invention that, upon administration to a recipient, is capable of providing, either directly or indirectly, a Compound of this invention or an inhibitory active metabolite or residue thereof.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of the Compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4alkyl)4 salts. The present invention also envisions the quaternization of any basic nitrogen-containing groups of the Compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the Compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutically acceptable compositions of this invention can be administered to humans and other animals orally, rectally, parenterally, intracistemally, intravaginally, intraperitoneally, topically (as by powders, ointments, or drops), bucally, as an oral or nasal spray, or the like, depending on the severity of the infection being treated. In certain embodiments, the compositions of the invention may be administered orally or parenterally, wherein the ENaC inhibitor compound and/or the CF Modulator modulator is/are present independently in the administered composition at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active Compounds of the composition, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a composition of the present invention, it is often desirable to slow the absorption of the composition from subcutaneous or intramuscular injection. This may be accomplished by the use of a liquid suspension of crystalline or amorphous material with poor water solubility. The rate of absorption of the composition then depends upon its rate of dissolution that, in turn, may depend upon crystal size and crystalline form. Alternatively, delayed absorption of a parenterally administered composition form is accomplished by dissolving or suspending the composition in an oil vehicle. Injectable depot forms are made by forming microencapsule matrices of the composition in biodegradable polymers such as polylactide-polyglycolide. Depending upon the ratio of composition to polymer and the nature of the particular polymer employed, the rate of composition release can be controlled. Examples of other biodegradable polymers include poly(orthoesters) and poly(anhydrides). Depot injectable formulations are also prepared by entrapping the composition in liposomes or microemulsions that are compatible with body tissues.

Compositions for rectal or vaginal administration are preferably suppositories which can be prepared by mixing the Compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active Compound.

Solid dosage forms for oral administration include capsules, tablets, mini-tablets, micro-tablets, particulates, micro and nano-particulates, pills, powders, and granules. In such solid dosage forms, the active Compound or combination of ENaC inhibitor and CF Modulator Compounds are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium Compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of capsules, tablets, mini-tablets, micro-tablets, particulates, micro and nano-particulates, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active Compound or combination of Compounds can also be in microencapsulated form with one or more excipients as noted above. The solid dosage forms of capsules, tablets, mini-tablets, micro-tablets, particulates, micro and nano-particulates, pills, and granules can be prepared with coatings and shells such as enteric coatings, release controlling coatings and other coatings well known in the pharmaceutical formulating art. In such solid dosage forms the active Compound or combination of compounds may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills; the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a Compound or combination of Compounds of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a Compound to the body. Such dosage forms are prepared by dissolving or dispensing the Compound in the proper medium. Absorption enhancers can also be used to increase the flux of the Compound across the skin. The rate can be controlled by either providing a rate controlling membrane or by dispersing the Compound in a polymer matrix or gel.

It will also be appreciated that the compositions disclosed herein can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive Compound or combination of Compounds may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

In one embodiment, the additional agent is selected from a mucolytic agent, bronchodialator, an anti-biotic, an anti-infective agent, an anti-inflammatory agent, a CFTR modulator other than a Compound of the present invention, or a nutritional agent.

In one embodiment, the additional agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In another embodiment, the additional agent is a mucolyte. Exemplary mucolytes useful herein includes Pulmozyme®.

In another embodiment, the additional agent is a bronchodialator. Exemplary bronchodialtors include albuterol, metaprotenerol sulfate, pirbuterol acetate, salmeterol, or tetrabuline sulfate.

In another embodiment, the additional agent is effective in restoring lung airway surface liquid. Such agents improve the movement of salt in and out of cells, allowing mucus in the lung airway to be more hydrated and, therefore, cleared more easily. Exemplary such agents include hypertonic saline, denufosol tetrasodium ([[(3S,5R)-5-(4-amino-2-oxopyrimidin-1-yl)-3-hydroxyoxolan-2-yl]methoxy-hydroxyphosphoryl][[[(2R,3S,4R,5R)-5-(2,4-dioxopyrimidin-1-yl)-3, 4-dihydroxyoxolan-2-yl]methoxy-hydroxyphosphoryl]oxy-hydroxyphosphoryl]hydrogen phosphate), or bronchitol (inhaled formulation of mannitol).

In another embodiment, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In another embodiment, the additional agent is a CFTR modulator other than the components disclosed in Columns A-D, i.e., an agent that has the effect of modulating CFTR activity. Exemplary such agents include ataluren (“PTC124®”; 3-[5-(2-fluorophenyl)-1,2,4-oxadiazol-3-yl]benzoic acid), sinapultide, lancovutide, depelestat (a human recombinant neutrophil elastase inhibitor), cobiprostone (7-{(2R,4aR,5R,7aR)-2-[(3S)-1,1-difluoro-3-methylpentyl]-2-hydroxy-6-oxooctahydrocyclopenta[b]pyran-5-yl}heptanoic acid), or (3-(6-(1-(2,2-difluorobenzo[d][1,3]dioxol-5-yl)cyclopropanecarboxamido)-3-methylpyridin-2-yl)benzoic acid. In another embodiment, the additional agent is (3-(6-(1-(2,2-difluorobenzo[d][1,3]dioxol-5-yl)cyclopropanecarboxamido)-3-methylpyridin-2-yl)benzoic acid.

In another embodiment, the additional agent is a nutritional agent. Exemplary such agents include pancrelipase (pancreating enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

A composition of the invention as disclosed herein may also be incorporated into compositions for coating an implantable medical device, such as prostheses, artificial valves, vascular grafts, stents and catheters. Accordingly, the present invention, in another aspect, includes a composition for coating an implantable device comprising a composition as disclosed herein or a pharmaceutically acceptable composition thereof, and in classes and subclasses herein, and a carrier suitable for coating said implantable device. In still another aspect, the present invention includes an implantable device coated with a composition comprising a composition as described herein or a pharmaceutically acceptable composition thereof, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

In order that the invention described herein may be more fully understood, the following examples are set forth. It should be understood that these examples are for illustrative purposes only and are not to be construed as limiting this invention in any manner.

For illustrative purposes only, formulations including any one CF modulator from Columns A, B, C, or D are intended as either single component formulations, or formulations containing the combination of CF Modulator modulator component from Columns A, B, C, or D and an ENaC inhibitor component from Column E

### III. Methods of Use

In yet another aspect, the present invention provides a method of treating a condition, disease, or disorder implicated by CFTR and/or ENaC dysfunction, the method comprising administering a pharmaceutical composition to a subject, preferably a mammal, in need thereof, the composition comprising a component from Column E (which includes an ENaC inhibitor, preferably an ENaC inhibitor that is a compound of Formula E) and at least one component from Columns A, B, C, and D according to Table I. In one embodiment, the pharmaceutical composition comprises an ENaC inhibitor from Column E and a at least one compound from Formulas A, B, C, or D. In another embodiment, the pharmaceutical composition comprises an ENaC inhibitor of Formula E and a compound of Formula A1. In one embodiment, the pharmaceutical composition comprises an ENaC inhibitor from Column E and a Compound of Formula C1. In one embodiment, the pharmaceutical composition comprises an ENaC inhibitor from Column E and a Compound of Formula D1. In another embodiment, the pharmaceutical composition comprises an ENaC inhibitor from Column E and Compound 1. In another embodiment, the pharmaceutical composition comprises an ENaC inhibitor from Column E and Compound 2. In another embodiment, the pharmaceutical composition comprises an ENaC inhibitor from Column E and Compound 3. In a further embodiment, the pharmaceutical composition comprises an ENaC inhibitor from Column E and a Compound 1 formulation. In a further embodiment, the pharmaceutical composition comprises an ENaC inhibitor from Column E and a Compound 2 formulation. In a further embodiment, the pharmaceutical composition comprises an ENaC inhibitor from Column E and a Compound 3 formulation.

In various embodiments, the administration of the combined active agents can be performed by administering each active agent of the combination as separate dosage units or as a single dosage unit. When administering the two active agents separately, each of the active agents can be administered concurrently, or one active agent can be administered prior to or after the other.

In certain embodiments, the present invention provides a method of treating a condition, disease, or disorder implicated by a deficiency of CFTR activity, the method comprising administering the pharmaceutical composition of the invention to a subject, preferably a mammal, in need thereof.

In yet another aspect, the present invention provides a method of treating, or lessening the severity of a condition, disease, or disorder implicated by CFTR mutation. In certain embodiments, the present invention provides a method of treating a condition, disease, or disorder implicated by a deficiency of the CFTR activity, the method comprising administering the pharmaceutical composition of the invention to a subject, preferably a mammal, in need thereof.

In another aspect, the invention also provides a method of treating or lessening the severity of a disease in a patient, the method comprising administering the pharmaceutical composition of the invention to a subject, preferably a mammal, in need thereof, and said disease is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear plasy, Pick's disease, several polyglutamine neurological disorders such as Huntington's, spinocerebullar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, or Sjogren's disease, Osteoporosis, Osteopenia, bone healing and bone growth (including bone repair, bone regeneration, reducing bone resorption and increasing bone deposition), Gorham's Syndrome, chloride channelopathies such as myotonia congenita (Thomson and Becker forms), Barter's syndrome type III, Dent's disease, hyperekplexia, epilepsy, hyperekplexia, lysosomal storage disease, Angelman syndrome, and Primary Ciliary Dyskinesia (PCD), a term for inherited disorders of the structure and/or function of cilia, including PCD with situs inversus (also known as Kartagener syndrome), PCD without situs inversus and ciliary aplasia.

In some embodiments, the method includes treating or lessening the severity of cystic fibrosis in a patient comprising administering to said patient one of the compositions as defined herein. In certain embodiments, the patient possesses mutant forms of human CFTR. In other embodiments, the patient possesses one or more of the following mutations ΔF508, R117H, and G551D of human CFTR. In one embodiment, the method includes treating or lessening the severity of cystic fibrosis in a patient possessing the ΔF508 mutation of human CFTR comprising administering to said patient one of the compositions as defined herein. In one embodiment, the method includes treating or lessening the severity of cystic fibrosis in a patient possessing the G551D mutation of human CFTR comprising administering to said patient one of the compositions as defined herein. In one embodiment, the method includes treating or lessening the severity of cystic fibrosis in a patient possessing the ΔF508 mutation of human CFTR on at least one allele comprising administering to said patient one of the compositions as defined herein. In one embodiment, the method includes treating or lessening the severity of cystic fibrosis in a patient possessing the ΔF508 mutation of human CFTR on both alleles comprising administering to said patient one of the compositions as defined herein. In one embodiment, the method includes treating or lessening the severity of cystic fibrosis in a patient possessing the G551D mutation of human CFTR on at least one allele comprising administering to said patient one of the compositions as defined herein. In one embodiment, the method includes treating or lessening the severity of cystic fibrosis in a patient possessing the G551D mutation of human CFTR on both alleles comprising administering to said patient one of the compositions as defined herein.

In some embodiments, the method includes lessening the severity of cystic fibrosis in a patient comprising administering to said patient one of the compositions as defined herein. In certain embodiments, the patient possesses mutant forms of human CFTR. In other embodiments, the patient possesses one or more of the following mutations ΔF508, R117H, and G551D of human CFTR. In one embodiment, the method includes lessening the severity of cystic fibrosis in a patient possessing the ΔF508 mutation of human CFTR comprising administering to said patient one of the compositions as defined herein. In one embodiment, the method includes lessening the severity of cystic fibrosis in a patient possessing the G551D mutation of human CFTR comprising administering to said patient one of the compositions as defined herein. In one embodiment, the method includes lessening the severity of cystic fibrosis in a patient possessing the ΔF508 mutation of human CFTR on at least one allele comprising administering to said patient one of the compositions as defined herein. In one embodiment, the method includes lessening the severity of cystic fibrosis in a patient possessing the ΔF508 mutation of human CFTR on both alleles comprising administering to said patient one of the compositions as defined herein. In one embodiment, the method includes lessening the severity of cystic fibrosis in a patient possessing the G551D mutation of human CFTR on at least one allele comprising administering to said patient one of the compositions as defined herein. In one embodiment, the method includes lessening the severity of cystic fibrosis in a patient possessing the G551D mutation of human CFTR on both alleles comprising administering to said patient one of the compositions as defined herein.

In some aspects, the invention provides a method of treating or lessening the severity of Osteoporosis in a patient comprising administering to said patient a composition as defined herein.

In certain embodiments, the method of treating or lessening the severity of Osteoporosis in a patient comprises administering to said patient a pharmaceutical composition as described herein.

In some aspects, the invention provides a method of treating or lessening the severity of Osteopenia in a patient comprising administering to said patient a composition as defined herein.

In certain embodiments, the method of treating or lessening the severity of Osteopenia in a patient comprises administering to said patient a pharmaceutical composition as described herein.

In some aspects, the invention provides a method of bone healing and/or bone repair in a patient comprising administering to said patient a composition as defined herein.

In certain embodiments, the method of bone healing and/or bone repair in a patient comprises administering to said patient a pharmaceutical composition as described herein.

In some aspects, the invention provides a method of reducing bone resorption in a patient comprising administering to said patient a composition as defined herein.

In some aspects, the invention provides a method of increasing bone deposition in a patient comprising administering to said patient a composition as defined herein.

In certain embodiments, the method of increasing bone deposition in a patient comprises administering to said patient a composition as defined herein.

In some aspects, the invention provides a method of treating or lessening the severity of COPD in a patient comprising administering to said patient a composition as defined herein.

In certain embodiments, the method of treating or lessening the severity of COPD in a patient comprises administering to said patient a composition as defined herein.

In some aspects, the invention provides a method of treating or lessening the severity of smoke induced COPD in a patient comprising administering to said patient a composition as defined herein.

In certain embodiments, the method of treating or lessening the severity of smoke induced COPD in a patient comprises administering to said patient a composition as defined herein.

In some aspects, the invention provides a method of treating or lessening the severity of chronic bronchitis in a patient comprising administering to said patient a composition as described herein.

In certain embodiments, the method of treating or lessening the severity of chronic bronchitis in a patient comprises administering to said patient a composition as defined herein.

According to an alternative embodiment, the present invention provides a method of treating cystic fibrosis comprising the step of administering to said mammal a composition as defined herein.

According to the invention an “effective amount” of the composition is that amount effective for treating or lessening the severity of one or more of the diseases, disorders or conditions as recited above.

Another aspect of the present invention provides a method of administering a pharmaceutical composition by orally administering to a patient at least once per day the composition as described herein. In one embodiment, the method comprises administering a composition to said patient a composition as defined herein once of Table I every 24 hours. In another embodiment, the method comprises administering to said patient a composition as defined herein every 12 hours. In a further embodiment, the method comprises administering a to said patient a composition as defined herein three times per day. In still a further embodiment, the method comprises administering to said patient a composition as defined herein.

The compositions, according to the method of the present invention, may be administered using any amount and any route of administration effective for treating or lessening the severity of one or more of the diseases, disorders or conditions as recited above.

In certain embodiments, the compositions of the present invention are useful for treating or lessening the severity of cystic fibrosis in patients who exhibit residual CFTR activity in the apical membrane of respiratory and non-respiratory epithelia. The presence of residual CFTR activity at the epithelial surface can be readily detected using methods known in the art, e.g., standard electrophysiological, biochemical, or histochemical techniques. Such methods identify CFTR activity using in vivo or ex vivo electrophysiological techniques, measurement of sweat or salivary Cl-concentrations, or ex vivo biochemical or histochemical techniques to monitor cell surface density. Using such methods, residual CFTR activity can be readily detected in patients heterozygous or homozygous for a variety of different mutations, including patients homozygous or heterozygous for the most common mutation, ΔF508.

In another embodiment, the compositions of the present invention are useful for treating or lessening the severity of cystic fibrosis in patients who have residual CFTR activity induced or augmented using pharmacological methods or gene therapy. Such methods increase the amount of CFTR present at the cell surface, thereby inducing a hitherto absent CFTR activity in a patient or augmenting the existing level of residual CFTR activity in a patient.

In one embodiment, a composition as defined herein can be useful for treating or lessening the severity of cystic fibrosis in patients within certain genotypes exhibiting residual CFTR activity, e.g., class III mutations (impaired regulation or gating), class IV mutations (altered conductance), or class V mutations (reduced synthesis) (Lee R. Choo-Kang, Pamela L., Zeitlin, Type I, II, III, IV, and V cystic fibrosis Transmembrane Conductance Regulator Defects and Opportunities of Therapy; Current Opinion in Pulmonary Medicine 6:521-529, 2000). Other patient genotypes that exhibit residual CFTR activity include patients homozygous for one of these classes or heterozygous with any other class of mutations, including class I mutations, class II mutations, or a mutation that lacks classification.

In one embodiment, a composition as defined herein can be useful for treating or lessening the severity of cystic fibrosis in patients within certain clinical phenotypes, e.g., a moderate to mild clinical phenotype that typically correlates with the amount of residual CFTR activity in the apical membrane of epithelia. Such phenotypes include patients exhibiting pancreatic insufficiency or patients diagnosed with idiopathic pancreatitis and congenital bilateral absence of the vas deferens, or mild lung disease.

The exact amount required will vary from subject to subject, depending on the species, age, and general condition of the subject, the severity of the infection, the particular agent, its mode of administration, and the like. The compositions of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compositions of the present invention will be decided by the attending physician within the scope of sound medical judgment. The specific effective dose level for any particular patient or organism will depend upon a variety of factors including the disorder being treated and the severity of the disorder; the activity of the composition employed; the specific composition employed; the age, body weight, general health, sex and diet of the patient; the time of administration, route of administration, and rate of excretion of the specific composition employed; the duration of the treatment; drugs used in combination or coincidental with the specific composition employed, and like factors well known in the medical arts. The term “patient”, as used herein, means an animal, preferably a mammal, and most preferably a human.

The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, each of the compounds used in the combination of the invention may be administered orally or parenterally at dosage levels of about 0.01 mg/kg to about 100 mg/kg and preferably from about 0.5 mg/kg to about 50 mg/kg, of subject body weight per day, one or more times a day, to obtain the desired therapeutic effect. In some embodiments, the unitary dose of each of the compounds can range from at least 0.1 mg/kg, at least 0.5 mg/kg, at least 1 mg/kg, at least 1.5 mg/kg, at least 5 mg/kg, at least 10 mg/kg, at least 15 mg/kg, at least 20 mg/kg, at least 30 mg/kg, at least 40 mg/kg, at least 50 mg/kg or at least 100 mg/kg. In some embodiments, each of the compounds formulated in a pharmaceutically acceptable composition can be administered alone or in combination to the subject in need or prophylactically in amounts ranging from about 0.1 to 1000 mg/day about 10 to 500 mg/day, for example 15, 30, 45 or 90, 100, 150, 200, 250, 300, 350, 400, or 450 mg/day

In some embodiments, the ratio of the ABC transporter modulator selected from Columns A-D to the ENaC inhibitor selected from Column E can range from 1000:1 to 1:1000, 500:1 to 1:500, 1:200 to 200:1, 100:1 to 1:100, 1:50 to 50:1, 25:1 to 1:25, 1:10 to 10:1 or from 1:5 to 5:1, preferrably, from 500:1, 400:1, 300,:1, 200:1, 100:1, 50:1, 25:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:100, 1:150, 1:200, 1:300, 1:400 or 1:500.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compounds to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, mini-tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as modulators of ABC transporters. Thus, without wishing to be bound by any particular theory, the compounds and compositions are particularly useful for treating or lessening the severity of a disease, condition, or disorder where hyperactivity or inactivity of ABC transporters is implicated in the disease, condition, or disorder. When hyperactivity or inactivity of an ABC transporter is implicated in a particular disease, condition, or disorder, the disease, condition, or disorder may also be referred to as a “ABC transporter-mediated disease, condition or disorder”. Accordingly, in another aspect, the present invention provides a method for treating or lessening the severity of a disease, condition, or disorder where hyperactivity or inactivity of an ABC transporter is implicated in the disease state.

The activity of a compound utilized in this invention as a modulator of an ABC transporter may be assayed according to methods described generally in the art and in the Examples herein.

It will also be appreciated that the compounds and pharmaceutically acceptable compositions of the present invention can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”. In some embodiments, the compounds can be administered as a single dose in one formulation e.g. a pill, tablet, capsule, trouche, granules, powdered, or solution comprising both compounds or the ABC transporter modulator selected from one or more of Columns A-D and a separate ENaC inhibitor compound from Column E can be administered to the subject in separate formulations, concurrently or sequentially.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention or pharmaceutically acceptable compositions thereof may also be incorporated into compositions for coating an implantable medical device, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

Another aspect of the invention relates to modulating ABC transporter activity and/or ENaC activity in a biological sample or a patient (e.g., in vitro or in vivo), which method comprises administering to the patient, or contacting said biological sample with a compound from Column A, and/or B and/or C and/or D and a compound from Column E to formulate the composition comprising said compounds. The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Modulation of ABC transporter activity and/or inhibition of ENaC activity in a biological sample is useful for a variety of purposes that are known to one of skill in the art. Examples of such purposes include, but are not limited to, the study of ABC transporters and ENaC activity in biological and pathological phenomena; and the comparative evaluation of new modulators of ABC transporters and/or inhibitors of ENaC activity.

In yet another embodiment, a method of modulating activity of an anion channel in vitro or in vivo, is provided comprising the step of contacting said channel with a combination composition comprising a compound from any one of Columns A and/or B, and/or C, and/or D and at least one compound from Column E. In some embodiments, the anion channel is a chloride channel or a bicarbonate channel. In other embodiments, the anion channel is a chloride channel.

According to an alternative embodiment, the present invention provides a method of increasing the number of functional ABC transporters in a membrane of a cell, comprising the step of contacting said cell with a combination composition comprising a compound from any of Columns A and/or B, and/or C, and/or D and at least one compound from Column E. The term “functional ABC transporter” as used herein means an ABC transporter that is capable of transport activity. In preferred embodiments, said functional ABC transporter is CFTR.

According to another preferred embodiment, the activity of the ABC transporter and/or ENaC activity is measured by measuring the transmembrane voltage potential. Means for measuring the voltage potential across a membrane in the biological sample may employ any of the known methods in the art, such as optical membrane potential assay or other electrophysiological methods.

The optical membrane potential assay utilizes voltage-sensitive FRET sensors described by Gonzalez and Tsien (See, Gonzalez, J. E. and R. Y. Tsien (1995) “Voltage sensing by fluorescence resonance energy transfer in single cells” Biophys J 69(4): 1272-80, and Gonzalez, J. E. and R. Y. Tsien (1997) “Improved indicators of cell membrane potential that use fluorescence resonance energy transfer” Chem Biol 4(4): 269-77) in combination with instrumentation for measuring fluorescence changes such as the Voltage/Ion Probe Reader (VIPR) (See, Gonzalez, J. E., K. Oades, et al. (1999) “Cell-based assays and instrumentation for screening ion-channel targets” Drug Discov Today 4(9): 431-439).

These voltage sensitive assays are based on the change in fluorescence resonant energy transfer (FRET) between the membrane-soluble, voltage-sensitive dye, DiSBAC2(3), and a fluorescent phospholipid, CC2-DMPE, which is attached to the outer leaflet of the plasma membrane and acts as a FRET donor. Changes in membrane potential (Vm) cause the negatively charged DiSBAC2(3) to redistribute across the plasma membrane and the amount of energy transfer from CC2-DMPE changes accordingly. The changes in fluorescence emission can be monitored using VIPR™ II, which is an integrated liquid handler and fluorescent detector designed to conduct cell-based screens in 96- or 384-well microtiter plates.

In another aspect the present invention provides a kit for use in measuring the activity of a ABC transporter or a fragment thereof in a biological sample in vitro or in vivo comprising (i) a combination composition comprising one or more compounds from any one of Columns A and/or B, and/or C, and/or D and at least one compound from Column E, or any of the above embodiments; and (ii) instructions for a.) contacting the composition with the biological sample and b.) measuring activity of said ABC transporter, a fragment thereof. and/or ENaC activity. In one embodiment, the kit further comprises instructions for a.) contacting an additional composition with the biological sample; b.) measuring the activity of said ABC transporter or a fragment thereof in the presence of said additional compound, and c.) comparing the activity of the ABC transporter in the presence of the additional compound with the density of the ABC transporter in the presence of a combination composition comprising one or more compounds from any one of Columns A and/or B, and/or C, and/or D and at least one compound from Column E. In preferred embodiments, the kit is used to measure the density of CFTR and/or ENaC.

While a number of embodiments and examples of this invention are described herein, it is apparent that these embodiments and examples may be altered to provide additional embodiments and examples which utilize the pharmaceutical formulations and drug regimens of this invention. Therefore, it will be appreciated that the scope of this invention is to be defined by the appended claims rather than by the specific embodiments that have been represented by way of example above.

In one aspect, the present invention features a kit comprising a composition as defined herein.

### IV Assays

### A. Protocol 1

Assays for Detecting and Measuring ΔF508-CFTR Potentiation Properties of Compounds

Membrane potential optical methods for assaying ΔF508-CFTR modulation properties of compounds The assay utilizes fluorescent voltage sensing dyes to measure changes in membrane potential using a fluorescent plate reader (e.g., FLIPR III, Molecular Devices, Inc.) as a readout for increase in functional ΔF508-CFTR in NIH 3T3 cells. The driving force for the response is the creation of a chloride ion gradient in conjunction with channel activation by a single liquid addition step after the cells have previously been treated with compounds and subsequently loaded with a voltage sensing dye.

Identification of Potentiator Compounds

To identify potentiators of ΔF508-CFTR, a double-addition HTS assay format was developed. This HTS assay utilizes fluorescent voltage sensing dyes to measure changes in membrane potential on the FLIPR III as a measurement for increase in gating (conductance) of ΔF508 CFTR in temperature-corrected ΔF508 CFTR NIH 3T3 cells. The driving force for the response is a Cl-ion gradient in conjunction with channel activation with forskolin in a single liquid addition step using a fluorescent plate reader such as FLIPR III after the cells have previously been treated with potentiator compounds (or DMSO vehicle control) and subsequently loaded with a redistribution dye.

Solutions

Bath Solution #1: (in mM) NaCl 160, KCl 4.5, CaCl2 2, MgCl 2 1, HEPES 10, pH 7.4 with NaOH.

Chloride-free bath solution: Chloride salts in Bath Solution #1 (above) are substituted with gluconate salts.

Cell Culture

NIH3T3 mouse fibroblasts stably expressing ΔF508-CFTR are used for optical measurements of membrane potential. The cells are maintained at 37° C. in 5% CO2 and 90% humidity in Dulbecco's modified Eagle's medium supplemented with 2 mM glutamine, 10% fetal bovine serum, 1 X NEAA, -ME, 1× pen/strep, and 25 mM HEPES in 175 cm2 culture flasks. For all optical assays, the cells were seeded at ˜20,000/well in 384-well matrigel-coated plates and cultured for 2 hrs at 37° C. before culturing at 27° C. for 24 hrs. for the potentiator assay. For the correction assays, the cells are cultured at 27° C. or 37° C. with and without compounds for 16-24 hours.

Electrophysiological Assays for assaying ΔF508-CFTR modulation properties of compounds.

Using Chamber Assay

Using chamber experiments were performed on polarized airway epithelial cells expressing ΔF508-CFTR to further characterize the ΔF508-CFTR modulators identified in the optical assays. Non-CF and CF airway epithelia were isolated from bronchial tissue, cultured as previously described (Galietta, L. J. V., Lantero, S., Gazzolo, A., Sacco, O., Romano, L., Rossi, G. A., & Zegarra-Moran, O. (1998) In Vitro Cell. Dev. Biol. 34, 478-481), and plated onto Costar® Snapwell™ filters that were precoated with NIH3T3-conditioned media. After four days the apical media was removed and the cells were grown at an air liquid interface for >14 days prior to use. This resulted in a monolayer of fully differentiated columnar cells that were ciliated, features that are characteristic of airway epithelia. Non-CF HBE were isolated from non-smokers that did not have any known lung disease. CF-HBE were isolated from patients homozygous for ΔF508-CFTR.

HBE grown on Costar® Snapwell™ cell culture inserts were mounted in an Using chamber (Physiologic Instruments, Inc., San Diego, Calif.), and the transepithelial resistance and short-circuit current in the presence of a basolateral to apical Cl-gradient (ISC) were measured using a voltage-clamp system (Department of Bioengineering, University of Iowa, IA). Briefly, HBE were examined under voltage-clamp recording conditions (Vhold=0 mV) at 37° C. The basolateral solution contained (in mM) 145 NaCl, 0.83 K2HPO4, 3.3 KH2PO4, 1.2 MgCl2, 1.2 CaCl2, 10 Glucose, 10 HEPES (pH adjusted to 7.35 with NaOH) and the apical solution contained (in mM) 145 NaGluconate, 1.2 MgCl2, 1.2 CaCl2, 10 glucose, 10 HEPES (pH adjusted to 7.35 with NaOH).

Identification of Potentiator Compounds

Typical protocol utilized a basolateral to apical membrane Cl-concentration gradient. To set up this gradient, normal ringers was used on the basolateral membrane, whereas apical NaCl was replaced by equimolar sodium gluconate (titrated to pH 7.4 with NaOH) to give a large Cl-concentration gradient across the epithelium. Forskolin (10 μM) and all test compounds were added to the apical side of the cell culture inserts. The efficacy of the putative ΔF508-CFTR potentiators was compared to that of the known potentiator, genistein.

Patch-Clamp Recordings

Total Cl-current in ΔF508-NIH3T3 cells was monitored using the perforated-patch recording configuration as previously described (Rae, J., Cooper, K., Gates, P., & Watsky, M. (1991) J. Neurosci. Methods 37, 15-26). Voltage-clamp recordings were performed at 22° C. using an Axopatch 200B patch-clamp amplifier (Axon Instruments Inc., Foster City, Calif.). The pipette solution contained (in mM) 150 N-methyl-d-glucamine (NMDG)-Cl, 2 MgCl2, 2 CaCl2, 10 EGTA, 10 HEPES, and 240 μg/mL amphotericin-B (pH adjusted to 7.35 with HCl). The extracellular medium contained (in mM) 150 NMDG-Cl, 2 MgCl2, 2 CaCl2, 10 HEPES (pH adjusted to 7.35 with HCl). Pulse generation, data acquisition, and analysis were performed using a PC equipped with a Digidata 1320 A/D interface in conjunction with Clampex 8 (Axon Instruments Inc.). To activate ΔF508-CFTR, 1011M forskolin and 20 genistein were added to the bath and the current-voltage relation was monitored every 30 sec.

Identification of Potentiator Compounds

The ability of ΔF508-CFTR potentiators to increase the macroscopic ΔF508-CFTR Cl-current (IΔF508) in NIH3T3 cells stably expressing ΔF508-CFTR was also investigated using perforated-patch-recording techniques. The potentiators identified from the optical assays evoked a dose-dependent increase in IΔF508 with similar potency and efficacy observed in the optical assays. In all cells examined, the reversal potential before and during potentiator application was around −30 mV, which is the calculated ECl (−28 mV).

Cell Culture

NIH3T3 mouse fibroblasts stably expressing ΔF508-CFTR are used for whole-cell recordings. The cells are maintained at 37° C. in 5% CO2 and 90% humidity in Dulbecco's modified Eagle's medium supplemented with 2 mM glutamine, 10% fetal bovine serum, 1×NEAA, -ME, 1× pen/strep, and 25 mM HEPES in 175 cm2 culture flasks. For whole-cell recordings, 2,500-5,000 cells were seeded on poly-L-lysine-coated glass coverslips and cultured for 24-48 hrs at 27° C. before use to test the activity of potentiators; and incubated with or without the correction compound at 37° C. for measuring the activity of correctors.

Single-Channel Recordings

Gating activity of wt-CFTR and temperature-corrected ΔF508-CFTR expressed in NIH3T3 cells was observed using excised inside-out membrane patch recordings as previously described (Dalemans, W., Barbry, P., Champigny, G., Jallat, S., Dott, K., Dreyer, D., Crystal, R. G., Pavirani, A., Lecocq, J-P., Lazdunski, M. (1991) Nature 354, 526-528) using an Axopatch 200B patch-clamp amplifier (Axon Instruments Inc.). The pipette contained (in mM): 150 NMDG, 150 aspartic acid, 5 CaCl2, 2 MgCl2, and 10 HEPES (pH adjusted to 7.35 with Tris base). The bath contained (in mM): 150 NMDG-Cl, 2 MgCl2, 5 EGTA, 10 TES, and 14 Tris base (pH adjusted to 7.35 with HCl). After excision, both wt- and ΔF508-CFTR were activated by adding 1 mM Mg-ATP, 75 nM of the catalytic subunit of cAMP-dependent protein kinase (PKA; Promega Corp. Madison, Wis.), and 10 mM NaF to inhibit protein phosphatases, which prevented current rundown. The pipette potential was maintained at 80 mV. Channel activity was analyzed from membrane patches containing ≦2 active channels. The maximum number of simultaneous openings determined the number of active channels during the course of an experiment. To determine the single-channel current amplitude, the data recorded from 120 sec of ΔF508-CFTR activity was filtered “off-line” at 100 Hz and then used to construct all-point amplitude histograms that were fitted with multigaussian functions using Bio-Patch Analysis software (Bio-Logic Comp. France). The total microscopic current and open probability (Po) were determined from 120 sec of channel activity. The Po was determined using the Bio-Patch software or from the relationship Po=I/i(N), where I=mean current, i=single-channel current amplitude, and N=number of active channels in patch.

Cell Culture

NIH3T3 mouse fibroblasts stably expressing ΔF508-CFTR are used for excised-membrane patch-clamp recordings. The cells are maintained at 37° C. in 5% CO2 and 90% humidity in Dulbecco's modified Eagle's medium supplemented with 2 mM glutamine, 10% fetal bovine serum, 1×NEAA, -ME, 1× pen/strep, and 25 mM HEPES in 175 cm2 culture flasks. For single channel recordings, 2,500-5,000 cells were seeded on poly-L-lysine-coated glass coverslips and cultured for 24-48 hrs at 27° C. before use.

Activity of the Compound 1

Compounds of the invention are useful as modulators of ATP binding cassette transporters. Table IV.A-1 below illustrates the EC50 and relative efficacy of certain embodiments in Table I. In Table IV.A-1 below, the following meanings apply. EC50. “+++” means <10 uM; “++” means between 10 uM to 25 uM; “+” means between 25 uM to 60 uM. % Efficacy: “+” means <25%; “++” means between 25% to 100%, “+++” means >100%.

TABLE IV
A-1

| Cmpd # | EC50 (uM) | % Activity |
|---|---|---|
| 1 | +++ | ++ |

### B. Protocol 2

Assays for Detecting and Measuring ΔF508-CFTR Correction Properties of Compounds

Membrane potential optical methods for assaying ΔF508-CFTR modulation properties of compounds.

The optical membrane potential assay utilized voltage-sensitive FRET sensors described by Gonzalez and Tsien (See Gonzalez, J. E. and R. Y. Tsien (1995) “Voltage sensing by fluorescence resonance energy transfer in single cells” Biophys J 69(4): 1272-80, and Gonzalez, J. E. and R. Y. Tsien (1997) “Improved indicators of cell membrane potential that use fluorescence resonance energy transfer” Chem Biol 4(4): 269-77) in combination with instrumentation for measuring fluorescence changes such as the Voltage/Ion Probe Reader (VIPR) (See, Gonzalez, J. E., K. Oades, et al. (1999) “Cell-based assays and instrumentation for screening ion-channel targets” Drug Discov Today 4(9): 431-439).

These voltage sensitive assays are based on the change in fluorescence resonant energy transfer (FRET) between the membrane-soluble, voltage-sensitive dye, DiSBAC2(3), and a fluorescent phospholipid, CC2-DMPE, which is attached to the outer leaflet of the plasma membrane and acts as a FRET donor. Changes in membrane potential (Vm) cause the negatively charged DiSBAC2(3) to redistribute across the plasma membrane and the amount of energy transfer from CC2-DMPE changes accordingly. The changes in fluorescence emission were monitored using VIPR™ II, which is an integrated liquid handler and fluorescent detector designed to conduct cell-based screens in 96- or 384-well microtiter plates.

Identification of Correction Compounds

To identify small molecules that correct the trafficking defect associated with ΔF508-CFTR; a single-addition HTS assay format was developed. The cells were incubated in serum-free medium for 16 hrs at 37° C. in the presence or absence (negative control) of test compound. As a positive control, cells plated in 384-well plates were incubated for 16 hrs at 27° C. to “temperature-correct” ΔF508-CFTR. The cells were subsequently rinsed 3× with Krebs Ringers solution and loaded with the voltage-sensitive dyes. To activate ΔF508-CFTR, 10 μM forskolin and the CFTR potentiator, genistein (20 μM), were added along with Cl-free medium to each well. The addition of Cl-free medium promoted Cl-efflux in response to ΔF508-CFTR activation and the resulting membrane depolarization was optically monitored using the FRET-based voltage-sensor dyes.

Identification of Potentiator Compounds

To identify potentiators of ΔF508-CFTR, a double-addition HTS assay format was developed. During the first addition, a Cl-free medium with or without test compound was added to each well. After 22 sec, a second addition of Cl-free medium containing 2-10 μM forskolin was added to activate ΔF508-CFTR. The extracellular Cl⁻ concentration following both additions was 28 mM, which promoted Cl⁻ efflux in response to ΔF508-CFTR activation and the resulting membrane depolarization was optically monitored using the FRET-based voltage-sensor dyes.

Solutions

Bath Solution #1: (in mM) NaCl 160, KCl 4.5, CaCl2 2, MgCl2 1, HEPES 10, pH 7.4 with NaOH.

Chloride-free bath solution: Chloride salts in Bath Solution #1 (above) are substituted with gluconate salts.

CC2-DMPE: Prepared as a 10 mM stock solution in DMSO and stored at −20° C.

DiSBAC2(3): Prepared as a 10 mM stock in DMSO and stored at −20° C.

Cell Culture

NIH3T3 mouse fibroblasts stably expressing ΔF508-CFTR are used for optical measurements of membrane potential. The cells are maintained at 37° C. in 5% CO2 and 90% humidity in Dulbecco's modified Eagle's medium supplemented with 2 mM glutamine, 10% fetal bovine serum, 1 X NEAA, β-ME, 1× pen/strep, and 25 mM HEPES in 175 cm2 culture flasks. For all optical assays, the cells were seeded at 30,000/well in 384-well matrigel-coated plates and cultured for 2 hrs at 37° C. before culturing at 27° C. for 24 hrs for the potentiator assay. For the correction assays, the cells are cultured at 27° C. or 37° C. with and without compounds for 16-24 hours.

Electrophysiological Assays for Assaying ΔF508-CFTR Modulation Properties of Compounds

Using Chamber Assay

Using chamber experiments were performed on polarized epithelial cells expressing ΔF508-CFTR to further characterize the ΔF508-CFTR modulators identified in the optical assays. FRTΔF508-CFTR epithelial cells grown on Costar Snapwell cell culture inserts were mounted in an Using chamber (Physiologic Instruments, Inc., San Diego, Calif.), and the monolayers were continuously short-circuited using a Voltage-clamp System (Department of Bioengineering, University of Iowa, IA, and, Physiologic Instruments, Inc., San Diego, Calif.). Transepithelial resistance was measured by applying a 2-mV pulse. Under these conditions, the FRT epithelia demonstrated resistances of 4 KΩ/cm2 or more. The solutions were maintained at 27° C. and bubbled with air. The electrode offset potential and fluid resistance were corrected using a cell-free insert. Under these conditions, the current reflects the flow of Cl-through ΔF508-CFTR expressed in the apical membrane. The ISC was digitally acquired using an MP100A-CE interface and AcqKnowledge software (v3.2.6; BIOPAC Systems, Santa Barbara, Calif.).

Identification of Correction Compounds

Typical protocol utilized a basolateral to apical membrane Cl-concentration gradient. To set up this gradient, normal ringer was used on the basolateral membrane, whereas apical NaCl was replaced by equimolar sodium gluconate (titrated to pH 7.4 with NaOH) to give a large Cl-concentration gradient across the epithelium. All experiments were performed with intact monolayers. To fully activate ΔF508-CFTR, forskolin (10 μM) and the PDE inhibitor, IBMX (100 μM), were applied followed by the addition of the CFTR potentiator, genistein (50 μM).

As observed in other cell types, incubation at low temperatures of FRT cells stably expressing ΔF508-CFTR increases the functional density of CFTR in the plasma membrane. To determine the activity of correction compounds, the cells were incubated with 10 μM of the test compound for 24 hours at 37° C. and were subsequently washed 3× prior to recording. The cAMP- and genistein-mediated ISC in compound-treated cells was normalized to the 27° C. and 37° C. controls and expressed as percentage activity. Preincubation of the cells with the correction compound significantly increased the cAMP- and genistein-mediated ISC compared to the 37° C. controls.

Identification of Potentiator Compounds

Typical protocol utilized a basolateral to apical membrane Cl-concentration gradient. To set up this gradient, normal ringers was used on the basolateral membrane and was permeabilized with nystatin (360 μg/ml), whereas apical NaCl was replaced by equimolar sodium gluconate (titrated to pH 7.4 with NaOH) to give a large Cl-concentration gradient across the epithelium. All experiments were performed 30 min after nystatin permeabilization. Forskolin (10 μM) and all test compounds were added to both sides of the cell culture inserts. The efficacy of the putative ΔF508-CFTR potentiators was compared to that of the known potentiator, genistein.

Solutions

Basolateral solution (in mM): NaCl (135), CaCl2 (1.2), MgCl2 (1.2), K2HPO4 (2.4), KHPO4 (0.6), N-2-hydroxyethylpiperazine-N′-2-ethanesulfonic acid (HEPES) (10), and dextrose (10). The solution was titrated to pH 7.4 with NaOH.

Apical solution (in mM): Same as basolateral solution with NaCl replaced with Na Gluconate (135).

Cell Culture

Fisher rat epithelial (FRT) cells expressing ΔF508-CFTR (FRTΔF508-CFTR) were used for Using chamber experiments for the putative ΔF508-CFTR modulators identified from our optical assays. The cells were cultured on Costar Snapwell cell culture inserts and cultured for five days at 37° C. and 5% CO2 in Coon's modified Ham's F-12 medium supplemented with 5% fetal calf serum, 100 U/ml penicillin, and 100 μg/ml streptomycin. Prior to use for characterizing the potentiator activity of compounds, the cells were incubated at 27° C. for 16-48 hrs to correct for the ΔF508-CFTR. To determine the activity of corrections compounds, the cells were incubated at 27° C. or 37° C. with and without the compounds for 24 hours.

Whole-Cell Recordings

The macroscopic ΔF508-CFTR current (IΔF508) in temperature- and test compound-corrected NIH3T3 cells stably expressing ΔF508-CFTR were monitored using the perforated-patch, whole-cell recording. Briefly, voltage-clamp recordings of IΔF508 were performed at room temperature using an Axopatch 200B patch-clamp amplifier (Axon Instruments Inc., Foster City, Calif.). All recordings were acquired at a sampling frequency of 10 kHz and low-pass filtered at 1 kHz. Pipettes had a resistance of 5-6 MΩ when filled with the intracellular solution. Under these recording conditions, the calculated reversal potential for Cl-(ECl) at room temperature was −28 mV. All recordings had a seal resistance >20 GΩ and a series resistance <15 Ma Pulse generation, data acquisition, and analysis were performed using a PC equipped with a Digidata 1320 A/D interface in conjunction with Clampex 8 (Axon Instruments Inc.). The bath contained <250 μL of saline and was continuously perfused at a rate of 2 ml/min using a gravity-driven perfusion system,

Identification of Correction Compounds

To determine the activity of correction compounds for increasing the density of functional ΔF508-CFTR in the plasma membrane, we used the above-described perforated-patch-recording techniques to measure the current density following 24-hr treatment with the correction compounds. To fully activate ΔF508-CFTR, 10 μM forskolin and 20 μM genistein were added to the cells. Under our recording conditions, the current density following 24-hr incubation at 27° C. was higher than that observed following 24-hr incubation at 37° C. These results are consistent with the known effects of low-temperature incubation on the density of ΔF508-CFTR in the plasma membrane. To determine the effects of correction compounds on CFTR current density, the cells were incubated with 10 μM of the test compound for 24 hours at 37° C. and the current density was compared to the 27° C. and 37° C. controls (% activity). Prior to recording, the cells were washed 3× with extracellular recording medium to remove any remaining test compound. Preincubation with 10 μM of correction compounds significantly increased the cAMP- and genistein-dependent current compared to the 37° C. controls.

Identification of Potentiator Compounds

The ability of ΔF508-CFTR potentiators to increase the macroscopic ΔF508-CFTR Cl-current (IΔF508) in NIH3T3 cells stably expressing ΔF508-CFTR was also investigated using perforated-patch-recording techniques. The potentiators identified from the optical assays evoked a dose-dependent increase in IΔF508 with similar potency and efficacy observed in the optical assays. In all cells examined, the reversal potential before and during potentiator application was around −30 mV, which is the calculated EC1 (−28 mV).

Solutions

Intracellular solution (in mM): Cs-aspartate (90), CsCl (50), MgCl2 (1), HEPES (10), and 240 μg/ml amphotericin-B (pH adjusted to 7.35 with CsOH).

Extracellular solution (in mM): N-methyl-d-glucamine (NMDG)-Cl (150), MgCl2 (2), CaCl2 (2), HEPES (10) (pH adjusted to 7.35 with HCl).

Cell Culture

NIH3T3 mouse fibroblasts stably expressing ΔF508-CFTR are used for whole-cell recordings. The cells are maintained at 37° C. in 5% CO2 and 90% humidity in Dulbecco's modified Eagle's medium supplemented with 2 mM glutamine, 10% fetal bovine serum, 1×NEAA, β-ME, 1× pen/strep, and 25 mM HEPES in 175 cm2 culture flasks. For whole-cell recordings, 2,500-5,000 cells were seeded on poly-L-lysine-coated glass coverslips and cultured for 24-48 hrs at 27° C. before use to test the activity of potentiators; and incubated with or without the correction compound at 37° C. for measuring the activity of correctors.

Single-Channel Recordings

The single-channel activities of temperature-corrected ΔF508-CFTR stably expressed in NIH3T3 cells and activities of potentiator compounds were observed using excised inside-out membrane patch. Briefly, voltage-clamp recordings of single-channel activity were performed at room temperature with an Axopatch 200B patch-clamp amplifier (Axon Instruments Inc.). All recordings were acquired at a sampling frequency of 10 kHz and low-pass filtered at 400 Hz. Patch pipettes were fabricated from Corning Kovar Sealing #7052 glass (World Precision Instruments, Inc., Sarasota, Fla.) and had a resistance of 5-8 MΩ when filled with the extracellular solution. The ΔF508-CFTR was activated after excision, by adding 1 mM Mg-ATP, and 75 nM of the cAMP-dependent protein kinase, catalytic subunit (PKA; Promega Corp. Madison, Wis.). After channel activity stabilized, the patch was perfused using a gravity-driven microperfusion system. The inflow was placed adjacent to the patch, resulting in complete solution exchange within 1-2 sec. To maintain ΔF508-CFTR activity during the rapid perfusion, the nonspecific phosphatase inhibitor F— (10 mM NaF) was added to the bath solution. Under these recording conditions, channel activity remained constant throughout the duration of the patch recording (up to 60 min). Currents produced by positive charge moving from the intra- to extracellular solutions (anions moving in the opposite direction) are shown as positive currents. The pipette potential (Vp) was maintained at 80 mV.

Channel activity was analyzed from membrane patches containing ≦2 active channels. The maximum number of simultaneous openings determined the number of active channels during the course of an experiment. To determine the single-channel current amplitude, the data recorded from 120 sec of ΔF508-CFTR activity was filtered “off-line” at 100 Hz and then used to construct all-point amplitude histograms that were fitted with multigaussian functions using Bio-Patch Analysis software (Bio-Logic Comp. France). The total microscopic current and open probability (Po) were determined from 120 sec of channel activity. The Po was determined using the Bio-Patch software or from the relationship Po=I/i(N), where I=mean current, i=single-channel current amplitude, and N=number of active channels in patch.

Solutions

Extracellular solution (in mM): NMDG (150), aspartic acid (150), CaCl2 (5), MgCl2 (2), and HEPES (10) (pH adjusted to 7.35 with Tris base).

Intracellular solution (in mM): NMDG-Cl (150), MgCl2 (2), EGTA (5), TES (10), and Tris base (14) (pH adjusted to 7.35 with HCl).

Cell Culture

NIH3T3 mouse fibroblasts stably expressing ΔF508-CFTR are used for excised-membrane patch-clamp recordings. The cells are maintained at 37° C. in 5% CO2 and 90% humidity in Dulbecco's modified Eagle's medium supplemented with 2 mM glutamine, 10% fetal bovine serum, 1×NEAA, -ME, 1× pen/strep, and 25 mM HEPES in 175 cm2 culture flasks. For single channel recordings, 2,500-5,000 cells were seeded on poly-L-lysine-coated glass coverslips and cultured for 24-48 hrs at 27° C. before use.

Using the procedures described above, the activity, (EC50), of Compound 2 has been measured and is shown in following Table VI.A-2

TABLE IV
A-2
IC50/EC50 Bins: +++ <= 2.0 < ++ <= 5.0 < +
Percent Activity Bins: + <= 25.0 < ++ <= 100.0 < +++

| | | |
|---|---|---|
| Cmpd. | Binned EC50 | Binned MaxEfficacy |
| Compound 2 | +++ | +++ |

### C. Protocol 3

Assays for Detecting and Measuring ΔF508-CFTR Correction Properties of Compounds

Membrane potential optical methods for assaying ΔF508-CFTR modulation properties of compounds.

The optical membrane potential assay utilized voltage-sensitive FRET sensors described by Gonzalez and Tsien (See Gonzalez, J. E. and R. Y. Tsien (1995) “Voltage sensing by fluorescence resonance energy transfer in single cells” Biophys J 69(4): 1272-80, and Gonzalez, J. E. and R. Y. Tsien (1997) “Improved indicators of cell membrane potential that use fluorescence resonance energy transfer” Chem Biol 4(4): 269-77) in combination with instrumentation for measuring fluorescence changes such as the Voltage/Ion Probe Reader (VIPR) (See, Gonzalez, J. E., K. Oades, et al. (1999) “Cell-based assays and instrumentation for screening ion-channel targets” Drug Discov Today 4(9): 431-439).

These voltage sensitive assays are based on the change in fluorescence resonant energy transfer (FRET) between the membrane-soluble, voltage-sensitive dye, DiSBAC2(3), and a fluorescent phospholipid, CC2-DMPE, which is attached to the outer leaflet of the plasma membrane and acts as a FRET donor. Changes in membrane potential (Vm) cause the negatively charged DiSBAC2(3) to redistribute across the plasma membrane and the amount of energy transfer from CC2-DMPE changes accordingly. The changes in fluorescence emission were monitored using VIPR™ II, which is an integrated liquid handler and fluorescent detector designed to conduct cell-based screens in 96- or 384-well microtiter plates.

Identification of Correction Compounds

To identify small molecules that correct the trafficking defect associated with ΔF508-CFTR; a single-addition HTS assay format was developed. The cells were incubated in serum-free medium for 16 h at 37° C. in the presence or absence (negative control) of test compound. As a positive control, cells plated in 384-well plates were incubated for 16 h at 27° C. to “temperature-correct” ΔF508-CFTR. The cells were subsequently rinsed 3× with Krebs Ringers solution and loaded with the voltage-sensitive dyes. To activate ΔF508-CFTR, 10 μM forskolin and the CFTR potentiator, genistein (20 μM), were added along with Cl-free-medium to each well. The addition of Cl-free-medium promoted Cl-efflux in response to ΔF508-CFTR activation and the resulting membrane depolarization was optically monitored using the FRET-based voltage-sensor dyes.

Identification of Potentiator Compounds

To identify potentiators of ΔF508-CFTR, a double-addition HTS assay format was developed. During the first addition, a Cl-free-medium with or without test compound was added to each well. After 22 sec, a second addition of Cl-free-medium containing 2-10 μM forskolin was added to activate ΔF508-CFTR. The extracellular Cl-concentration following both additions was 28 mM, which promoted Cl-efflux in response to ΔF508-CFTR activation and the resulting membrane depolarization was optically monitored using the FRET-based voltage-sensor dyes.

Solutions

Bath Solution #1: (in mM) NAcl 160, KCl 4.5, CaCl2 2, MgCl2 1, HEPES 10, pH 7.4 with NaOH.

Chloride-free bath solution: Chloride salts in Bath Solution #1 (above) are substituted with gluconate salts.

CC2-DMPE: Prepared as a 10 mM stOCk solution in DMSO and stored at −20° C.

DiSBAC2(3): Prepared as a 10 mM stOCk in DMSO and stored at −20° C.

Cell Culture

NIH3T3 mouse fibroblasts stably expressing ΔF508-CFTR are used for optical measurements of membrane potential. The cells are maintained at 37° C. in 5% CO2 and 90% humidity in Dulbecco's modified Eagle's medium supplemented with 2 mM glutamine, 10% fetal bovine serum, 1 X NEAA, β-ME, 1× pen/strep, and 25 mM hepes in 175 cm2 culture flasks. For all optical assays, the cells were seeded at 30,000/well in 384-well matrigel-coated plates and cultured for 2 h at 37° C. before culturing at 27° C. for 24 h for the potentiator assay. For the correction assays, the cells are cultured at 27° C. or 37° C. with and without compounds for 16-24-ours.

Electrophysiological Assays for Assaying ΔF508-CFTR Modulation Properties of Compounds

Using Chamber Assay

Using chamber experiments were performed on polarized epithelial cells expressing ΔF508-CFTR to further characterize the ΔF508-CFTR modulators identified in the optical assays. FRTΔF508-CFTR epithelial cells grown on Costar Snapwell cell culture inserts were mounted in an Using chamber (Physiologic Instruments, Inc., San Diego, Calif.), and the monolayers were continuously short-circuited using a Voltage-clamp System (Department of Bioengineering, University of Iowa, IA, and, Physiologic Instruments, Inc., San Diego, Calif.). Transepithelial resistance was measured by applying a 2-mV pulse. Under these conditions, the FRT epithelia demonstrated resistances of 4 KΩ/cm2 or more. The solutions were maintained at 27° C. and bubbled with air. The electrode offset potential and fluid resistance were corrected using a cell-free insert. Under these conditions, the current reflects the flow of Cl-through ΔF508-CFTR expressed in the apical membrane. The ISC was digitally acquired using an MP100A-CE interface and AcqKnowledge software (v3.2.6; BIOPAC Systems, Santa Barbara, Calif.).

Identification of Correction Compounds

Typical protocol utilized a basolateral to apical membrane Cl-concentration gradient. To et up this gradient, normal ringer was used on the basolateral membrane, whereas apical NaCl was replaced by equimolar sodium gluconate (titrated to pH 7.4 with NaOH) to give a large Cl-concentration gradient across the epithelium. All experiments were performed with intact monolayers. To fully activate ΔF508-CFTR, forskolin (10 μM) and the PDE inhibitor, IBMX (100 μM), were applied followed by the addition of the CFTR potentiator, genistein (50 μM).

As observed in other cell types, incubation at low temperatures of FRT cells stably expressing ΔF508-CFTR increases the functional density of CFTR in the plasma membrane. To determine the activity of correction compounds, the cells were incubated with 10 μM of the test compound for 24 hours at 37° C. and were subsequently washed 3× prior to recording. The cAMP-AND genistein-mediated ISC in compound-treated cells was normalized to the 27° C. and 37° C. controls and expressed as percentage activity. Preincubation of the cells with the correction compound significantly increased the cAMP-AND genistein-mediated ISC compared to the 37° C. controls.

Identification of Potentiator Compounds

Typical protocol utilized a basolateral to apical membrane Cl-concentration gradient. To set up this gradient, normal ringers was used on the basolateral membrane and was permeabilized with nystatin (360 μg/mL), whereas apical NaCl was replaced by equimolar sodium gluconate (titrated to pH 7.4 with NaOH) to give a large Cl-concentration gradient across the epithelium. All experiments were performed 30 min after nystatin permeabilization. Forskolin (10 μM) and all test compounds were added to both sides of the cell culture inserts. The efficacy of the putative ΔF508-CFTR potentiators was compared to that of the known potentiator, genistein.

Solutions

Basolateral solution (in mM): NaCl (135), CaCl2 (1.2), MgCl2 (1.2), K2HPO4 (2.4), KHPO4 (0.6), N-2-hydroxyethylpiperazine-N′-2-ethanesulfonic acid (HEPES) (10), and dextrose (10). The solution was titrated to pH 7.4 with NaOH.

Apical solution (in mM): same as basolateral solution with NaCl replaced with Na Gluconate (135).

Cell Culture

Fisher rat epithelial (FRT) cells expressing ΔF508-CFTR (FRT ΔF508-CFTR) were used for Using chamber experiments for the putative ΔF508-CFTR modulators identified from our optical assays. The cells were cultured on Costar Snapwell cell culture inserts and cultured for five days at 37° C. and 5% CO2 in Coon's modified Ham's F-12 medium supplemented with 5% fetal calf serum, 100 U/mL penicillin, and 100 μg/mL streptomycin. Prior to use for characterizing the potentiator activity of compounds, the cells were incubated at 27° C. for 16-48-rs to correct for the ΔF508-CFTR. To determine the activity of corrections compounds, the cells were incubated at 27° C. or 37° C. with and without the compounds for 24 hours.

Whole-Cell Recordings

The macroscopic ΔF508-CFTR current (I ΔF508) in temperature- and test compound-corrected NIH3T3 cells stably expressing ΔF508-CFTR were monitored using the perforated-patch, whole-cell recording. Briefly, voltage-clamp recordings of IΔF508 were performed at room temperature using an Axopatch 200B patch-clamp amplifier (Axon Instruments Inc., Foster City, Calif.). All recordings were acquired at a sampling frequency of 10 kHz and low-pass filtered at 1 kHz. Pipettes had a resistance of 5-6 MΩ when filled with the intracellular solution. Under these recording conditions, the calculated reversal potential for Cl-(ECl) at room temperature was −28 mV. aLl recordings had a seal resistance >20 GΩ and a series resistance <15 MΩ Pulse generation, data acquisition, and analysis were performed using a PC equipped with a Digidata 1320 A/D interface in conjunction with Clampex 8 (Axon Instruments Inc.). The bath contained <250 μl of saline and was continuously perfused at a rate of 2 mL/min using a gravity-driven perfusion system,

Identification of Correction Compounds

To determine the activity of correction compounds for increasing the density of functional ΔF508-CFTR in the plasma membrane, we used the above-described perforated-patch-recording techniques to measure the current density following 24-h treatment with the correction compounds. To fully activate ΔF508-CFTR, 10 μM forskolin and 20 μM genistein were added to the cells. Under our recording conditions, the current density following 24-h incubation at 27° C. was higher than that observed following 24-h incubation at 37° C. These results are consistent with the known effects of low-temperature incubation on the density of ΔF508-CFTR in the plasma membrane. To determine the effects of correction compounds on CFTR current density, the cells were incubated with 10 μM of the test compound for 24 hours at 37° C. and the current density was compared to the 27° C. and 37° C. controls (% activity). Prior to recording, the cells were washed 3× with extracellular recording medium to remove any remaining test compound. Preincubation with 10 μM of correction compounds significantly increased the cAMP-AND genistein-dependent current compared to the 37° C. controls.

Identification of Potentiator Compounds

The ability of ΔF508-CFTR potentiators to increase the macroscopic ΔF508-CFTR Cl-current (I ΔF508) in NIH3T3 cells stably expressing ΔF508-CFTR was also investigated using perforated-patch-recording techniques. The potentiators identified from the optical assays evoked a dose-dependent increase in I ΔF508 with similar potency and efficacy observed in the optical assays. In all cells examined, the reversal potential before and during potentiator application was around −30 mV, which is the calculated EC1 (−28 mV).

Solutions

Intracellular solution (in mM): cs-aspartate (90), CsCl (50), MgCl2 (1), HEPES (10), and 240 μg/mL amphotericin-B (pH adjusted to 7.35 with CsOH).

Extracellular solution (in mM): n-methyl-d-glucamine (NMDG)-Cl (150), MgCl2 (2), CaCl2 (2), HEPES (10) (pH adjusted to 7.35 with HCl).

Cell Culture

NIH3T3 mouse fibroblasts stably expressing ΔF508-CFTR are used for whole-cell recordings. The cells are maintained at 37° C. in 5% CO2 and 90% humidity in Dulbecco's modified Eagle's medium supplemented with 2 mM glutamine, 10% fetal bovine serum, 1×NEAA, β-ME, 1× pen/strep, and 25 mM Hepes in 175 cm2 culture flasks. For whole-cell recordings, 2,500-5, 0-0 cells were seeded on poly-L-lysine-coated glass coverslips and cultured for 24-48-rs at 27° C. before use to test the activity of potentiators; and incubated with or without the correction compound at 37° C. for measuring the activity of correctors.

Single-Channel Recordings

The single-channel activities of temperature-corrected ΔF508-CFTR stably expressed in NIH3T3 cells and activities of potentiator compounds were observed using excised inside-out membrane patch. Briefly, voltage-clamp recordings of single-channel activity were performed at room temperature with an Axopatch 200B patch-clamp amplifier (Axon Instruments Inc.). All recordings were acquired at a sampling frequency of 10 kHz and low-pass filtered at 400 Hz. Patch pipettes were fabricated from Corning Kovar Sealing #7052 glass (World Precision Instruments, Inc., Sarasota, Fla.) and had a resistance of 5-8 M—when filled with the extracellular solution. The ΔF508-CFTR was activated after excision, by adding 1 mM Mg-ATP, and 75 nM of The cAMP-dependent protein kinase, catalytic subunit (PKA; Promega Corp. Madison, Wis.). After channel activity stabilized, the patch was perfused using a gravity-driven microperfusion system. The inflow was placed adjacent to the patch, resulting in complete solution exchange within 1-2 s-c. To maintain ΔF508-CFTR activity during the rapid perfusion, the nonspecific phosphatase inhibitor F—(10 mM NaF) was added to the bath solution. Under these recording conditions, channel activity remained constant throughout the duration of the patch recording (up to 60 min). Currents produced by positive charge moving from the intra- to extracellular solutions (anions moving in the opposite direction) are shown as positive currents. The pipette potential (Vp) was maintained at 80 mV.

Channel activity was analyzed from membrane patches containing ≦2 active channels. The maximum number of simultaneous openings determined the number of active channels during the course of an experiment. To determine the single-channel current amplitude, the data recorded from 120 sec of ΔF508-CFTR activity was filtered “off-line” at 100 Hz and then used to construct all-point amplitude histograms that were fitted with multigaussian functions using Bio-Patch Analysis software (Bio-Logic Comp. France). The total microscopic current and open probability (Po) were determined from 120 sec of channel activity. The Po was determined using the Bio-Patch software or from the relationship Po=I/i(N), where I=mean current, i=single-channel current amplitude, and N=number of active channels in patch.

Solutions

Extracellular solution (in mM): nm DG (150), aspartic acid (150), CaCl2 (5), MgCl2 (2), and HEPES (10) (pH adjusted to 7.35 with Tris base).

Intracellular solution (in mM): nMDG-Cl (150), MgCl2 (2), EGTA (5), TES (10), and Tris base (14) (pH adjusted to 7.35 with HCl).

Cell Culture

NIH3T3 mouse fibroblasts stably expressing ΔF508-CFTR are used for excised-membrane patch-clamp recordings. The cells are maintained at 37° C. in 5% CO2 and 90% humidity in Dulbecco's modified Eagle's medium supplemented with 2 mM glUTamine, 10% fetal bovine serum, 1 X NEAA, -ME, 1× pen/strep, and 25 mM Hepes in 175 cm2 culture flasks. For single channel recordings, 2,500-5,0-0 cells were seeded on poly-L-lysine-coated glass cover slips and cultured for 24-48-rs at 27° C. before use.

Using the procedures described above, the activity, i.e., EC50s, of Compound 3 has been measured and is shown in Table IV.A-3.

TABLE IV
A-3
IC50/EC50 Bins: +++ <= 2.0 < ++ <= 5.0 < +
Percent Activity Bins: + <= 25.0 < ++ <= 100.0 < +++

| | | |
|---|---|---|
| Cmpd. | Binned EC50 | Binned MaxEfficacy |
| Compound 3 | +++ | +++ |

### D. Protocol 4

Methods for testing the combined effects of CFTR and ENaC modulators on fluid transport in cultures of CF HBE.

To test combinations of CFTR modulators and pharmacological agents that reduce epithelial sodium channel (ENaC) activity either directly or indirectly on epithelial cell fluid transport, the height of the airway surface liquid (ASL) on the apical surface of human bronchial epithelial (HBE) cells obtained from the bronchi of CF patients was measured using confocal immunofluorescent microscopy. The apical surface was washed 2 times with 300 μl absorption buffer (89 mM NaCl, 4 mM KCl, 1.2 mM MgCl2, 1.2 mM CaCl2, 1 mM HEPES, 16 mM Na-Gluconate, 10 mM Glucose) pre warmed to 37° C. After the final wash, 20 μl of 10,000 Kd dextran conjugated to Alexa Fluor 488 in absorption buffer was added and allowed to equilibrate for 2 days prior to testing. To test the effect of pharmacological modulation on the ASL, CFTR modulators prepared in HBE differentiation media [Dulbeco's MEM (DMEM)/F12, Ultroser-G (2.0%; Pall Catalog #15950-017), Fetal Clone II (2%), Insulin (2.5 μg/ml), Bovine Brain Extract (0.25%; Lonza Kit #CC-4133, component #CC-4092C), Hydrocortisone (20 nM), Triodothyronine (500 nM), Transferrin (2.5 μg/ml: InVitrogen Catalog #0030124SA), Ethanolamine (250 nM), Epinephrine (1.5 μM), Phosphoethanolamine (250 nM), Retinoic acid (10 nM)] were applied to the basolateral side at desired concentration. ENaC modulators were prepared in 2000 μl of Fluorinert FC-770 (3M) at the final concentration and 100 μl of the solution was added to the apical surface. After 96 hours of treatment the ASL height was measured using a Quorum Wave FX Spinning Disc Confocal System on an Inverted Zeiss microscope and 20× objective. The images were acquired and processed using Volocity 4.0.

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed:

1. A pharmaceutical composition comprising:
A. an epithelial sodium channel (ENaC) inhibitor; and
B. at least one ABC transporter modulator, the ABC transporter modulator comprising:
I. a compound of Formula A: 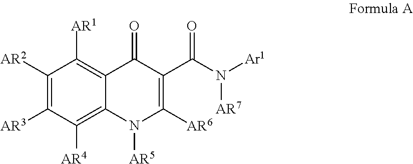
or a pharmaceutically acceptable salt thereof, wherein: Ar¹ is selected from: 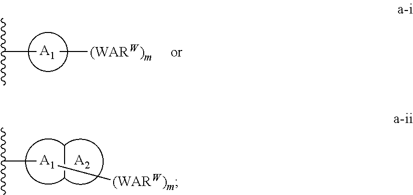
wherein ring A₁ 5-6 membered aromatic monocyclic ring having 0-4 heteroatoms independently selected from nitrogen, oxygen, or sulfur; or
A₁ and A₂, together, is an 8-14 aromatic, bicyclic or tricyclic aryl ring, wherein each ring contains 0-4 heteroatoms independently selected from nitrogen, oxygen, or sulfur;
W is a bond or is an optionally substituted C₁-C₆ alkylidene chain wherein up to two methylene units of W are optionally and independently replaced by —CO—, —CS—, —COCO—, —CON(AR′)—, —CON(AR′)N(AR′)—, —CO₂—, —OCO—, —N(AR′)CO₂—, —O—, —N(AR′)CON(AR′)—, —OCON(AR′)—, —N(AR′)N(AR′), —N(AR′)N(AR′)CO—, —N(AR′)CO—, —S—, —SO, —SO₂—, —N(AR′)—, —SO₂N(AR′)—, N(AR′)SO₂—, or —N(AR′)SO₂N(AR′)—;
AR^{W} is independently AR′, halo, NO₂, CN, CF₃, or OCF₃;
m is 0-5;
each of AR′, AR², AR³, AR⁴, and AR⁵ is independently —X-AR^{X};
X is a bond or is an optionally substituted C₁-C₆ alkylidene chain wherein up to two methylene units of X are optionally and independently replaced by —CO—, —CS—, —COCO—, —CON(AR′)—, —CON(AR′)N(AR′)—, —CO₂—, —OCO—, —N(AR′)CO₂—, —O—, —N(AR′)CON(AR′)—, —OCON(AR′)—, —N(AR′)N(AR′), —N(AR′)N(AR′)CO—, —N(AR′)CO—, —S—, —SO, —SO₂—, —N(AR′)—, —SO₂N(AR′)—, N(AR′)SO₂—, or —N(AR′)SO₂N(AR′)—;
AR^{X} is independently AR′, halo, NO₂, CN, CF₃, or OCF₃;
AR⁶ is hydrogen, CF₃, —OAR′, —SAR′, or an optionally substituted C₁₋₆ aliphatic group;
AR⁷ is hydrogen or a C₁₋₆ aliphatic group optionally substituted with —X-AR^{X}; and
AR′ is independently selected from hydrogen or an optionally substituted group selected from a C₁-C₈ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or two occurrences of AR′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur; or
II. a compound of Formula B: 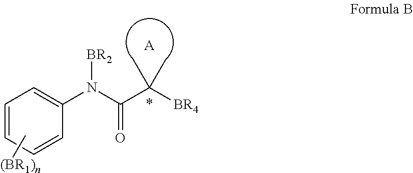
or a pharmaceutically acceptable salt thereof wherein: each BR₁ is an optionally substituted C₁₋₆ aliphatic, an optionally substituted aryl, an optionally substituted heteroaryl, an optionally substituted C₃₋₁₀ cycloaliphatic, or an optionally substituted 4 to 10 membered heterocycloaliphatic, carboxy [e.g., hydroxycarbonyl or alkoxycarbonyl], alkoxy, amido [e.g., aminocarbonyl], amino, halo, cyano, alkylsulfanyl, or hydroxy; provided that at least one R₁ is an optionally substituted aryl or an optionally substituted heteroaryl and said R₁ is attached to the 3- or 4-position of the phenyl ring; each BR₂ is hydrogen, an optionally substituted C₁₋₆ aliphatic, an optionally substituted C₃₋₆ cycloaliphatic, an optionally substituted phenyl, or an optionally substituted heteroaryl; each BR₄ is an optionally substituted aryl or an optionally substituted heteroaryl; each n is 1, 2, 3, 4 or 5; and ring A is an optionally substituted cycloaliphatic or an optionally substituted heterocycloaliphatic where the atoms of ring A adjacent to C* are carbon atoms, and each of which is optionally substituted with 1, 2, or 3 substituents; or
III. a compound of Formula C: 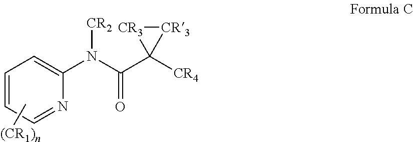
or a pharmaceutically acceptable salt thereof, wherein each CR₁ is a an optionally substituted C₁-C₆ aliphatic, an optionally substituted aryl, an optionally substituted heteroaryl, an optionally substituted 3 to 10 membered cycloaliphatic, an optionally substituted 3 to 10 membered heterocycloaliphatic, carboxy [e.g., hydroxycarbonyl or alkoxycarbonyl], amido, amino, halo, or hydroxy, provided that at least one R₁ is an optionally substituted aryl or an optionally substituted heteroaryl attached to the 5- or 6-position of the pyridyl ring, each R₂ is hydrogen, an optionally substituted C₁₋₆ aliphatic, an optionally substituted C₃₋₆ cycloaliphatic, an optionally substituted phenyl, or an optionally substituted heteroaryl, each CR₃ and CR′₃ together with the carbon atom to which they are attached form an optionally substituted C₃₋₇ cycloaliphatic or an optionally substituted heterocycloaliphatic, each CR₄ is an optionally substituted aryl or an optionally substituted heteroaryl, each n is 1-4; or
IV. a compound of Formula D: 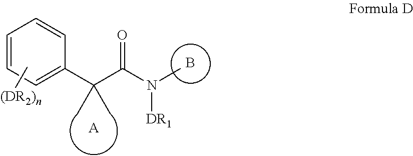
or a pharmaceutically acceptable salt thereof, wherein R₁ is —Z^{A}DR₄, and wherein each Z^{A} is independently a bond or an optionally substituted branched or straight C₁₋₆ aliphatic chain wherein up to two carbon units of Z^{A} are optionally and independently replaced by —CO—, —CS—, —CONDR^{A}—, —CONDR^{A}NDR^{A}—, —CO₂—, —OCO—, —NDR^{A}CO₂—, —O—, —NDR^{A}CONDR^{A}—, —OCONDR^{A}—, —NDR^{A}NDR^{A}—, —NDR^{A}CO—, —S—, —SO—, —SO₂—, —NDR^{A}—, —SO₂NDR^{A}—, —NDR^{A}SO₂—, or —NDR^{A}SO₂NDR^{A}—,
Each DR₄ is independently DR^{A}, halo, —OH, —NH₂, —NO₂, —CN, or —OCF₃, each DR^{A} is independently hydrogen, an optionally substituted aliphatic, an optionally substituted cycloaliphatic, an optionally substituted heterocycloaliphatic, an optionally substituted aryl, or an optionally substituted heteroaryl, DR₂ is —Z^{B}DR₅, and wherein each Z^{B} is independently a bond or an optionally substituted branched or straight C₁₋₆ aliphatic chain wherein up to two carbon units of Z^{B} are optionally and independently replaced by —CO—, —CS—, —CONDR^{B}—, —CONDR¹NDR^{B}—, —CO₂—, —OCO—, —NDR^{B}CO₂—, —O—, —NDR^{B}CONDR^{B}—, —OCONDR^{B}—, —NDR^{B}NDR^{B}—, —NDR^{B}CO—, —S—, —SO—, —SO₂—, —NDR^{B}—, —SO₂NDR^{B}—, —NDR^{B}SO₂—, or —NDR^{B}SO₂NDR^{B}—, each DR₅ is independently DR^{B}, halo, —OH, —NH₂, —NO₂, —CN, —CF₃, or —OCF₃,
Each DR^{B} is independently hydrogen, an optionally substituted aliphatic, an optionally substituted cycloaliphatic, an optionally substituted heterocycloaliphatic, an optionally substituted aryl, or an optionally substituted heteroaryl, and wherein any two adjacent R₂ groups together with the atoms to which they are attached form an optionally substituted carbocycle or an optionally substituted heterocycle,
wherein ring A is an optionally substituted 3-7 membered monocyclic ring having 0-3 heteroatoms selected from N, O, and S and ring B is a group having formula Ia

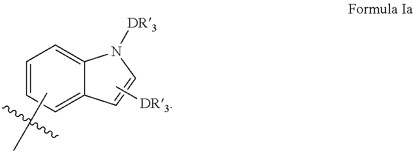

2. The pharmaceutical composition of claim 1, wherein the ENaC inhibitor is a compound of Formula E 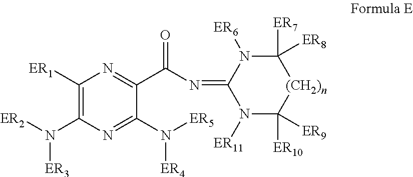
or pharmaceutically acceptable salts, solvates, hydrates thereof, wherein ER¹ is H, halogen, C₁-C₈-alkyl, C₁C₈-haloalkyl, C₁-C₈-haloalkoxy, C₃C₁₅-carbocyclic group, nitro, cyano, a C₆-C₁₅-membered aromatic carbocyclic group, or a C₁-C₈-alkyl substituted by a C₆-C₁₅-membered aromatic carbocyclic group;
ER², ER³, ER⁴ and ER⁵ are each independently selected from H and C₁-C₆ alkyl;
ER⁶, ER⁷, ER⁸, ER⁹, ER¹⁰ and ER¹¹ are each independently selected from H; SO₂ER¹⁶; aryl optionally substituted by one or more Z groups; a C₃-C₁₀ carbocyclic group optionally substituted by one or more Z groups; C₃-C₁₄ heterocyclic group optionally substituted by one or more Z groups; C₁-C₈ alkyl optionally substituted by an aryl group which is optionally substituted by one or more Z groups, a C₃-C₁₀ carbocyclic group optionally substituted by one or more Z groups or a C₃-C₁₄ heterocyclic group optionally substituted by one or more Z groups; or is represented by the Formula E2:
-(C₀-C₆ alkylene)-A-(C₀-C₆ alkylene)-B-(X-ER¹²)_{q}-ER²²,
wherein the alkylene groups are optionally substituted by one or more Z groups;
or ER⁶ and ER⁷ together with the atoms to which they are attached form a 3- to 10-membered heterocyclic group, the heterocyclic group including one or more further heteroatoms selected from N, O and S, and the heterocyclic group being optionally substituted by one or more Z groups; SO₂ER¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the formula 2;
or ER⁷ and ER⁸ together with the carbon atom to which they are attached form a 3- to 10-membered carbocyclic or a 3- to 10-membered heterocyclic group, the heterocyclic group including one or more heteroatoms selected from N, O and S, and the carbocyclic and heterocyclic groups being optionally substituted by one or more Z groups; SO₂R¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the formula 2;
or ER⁹ and ER¹⁰ together with the carbon atom to which they are attached form a 3- to 10-membered carbocyclic or a 3- to 10-membered heterocyclic group, the heterocyclic group including one or more heteroatoms selected from N, O and S, and the carbocyclic and heterocyclic groups being optionally substituted by one or more Z groups; SO₂ER¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups;
or a group represented by the Formula E2;
or ER⁸ and ER⁹ together with the carbon atoms to which they are attached form a 3- to 10-membered cycloalkyl or a 3- to 10-membered heterocyclic group, the heterocyclic group including one or more heteroatoms selected from N, O and S, and the carbocyclic and heterocyclic groups being optionally substituted by one or more Z groups; SO₂ER¹⁶;
C₆-C₁₅-aromatic carbocyclic group optionally substituted by one or more Z groups; a C₃-C₁₀ carbocyclic group; a C₃-C₁₄ heterocyclic group optionally substituted by one or more Z groups;
or a group represented by the formula 2;
or ER¹⁰ and ER¹¹ together with the atoms to which they are attached form a 3- to 10-membered heterocyclic group, the heterocyclic group including one or more further heteroatoms selected from N, O and S, and the heterocyclic group being optionally substituted by one or more Z groups; SO₂ER¹⁶; C₆-C₁₅-aromatic carbocyclic group optionally substituted by one or more Z groups; a C₃-C₁₀ carbocyclic group; a C₃-C₁₄ heterocyclic group optionally substituted by one or more Z groups; or a group represented by the formula 2;
A is selected from a bond, —NER¹³(SO₂)—, —(SO₂)NER¹³—, —(SO₂)—, —NER¹³C(O)—, —C(O)NER¹³—, —NER¹³C(O)NER¹⁴—, —NER¹³C(O)O—, —NER¹³—, C(O)O, OC(O), C(O), O and S;
B is selected from a bond, —(C₂-C₄ alkenyl group)-, —(C₂-C₄ alkynyl group)-, —NH—, aryl, O-aryl, NH-aryl, a C₃-C₁₄ carbocyclic group and a 3- to 14-membered heterocyclic group, the heterocyclic group including one or more heteroatoms selected from N, O and S, wherein the aryl, carbocyclic and heterocyclic groups are each optionally substituted by one or more Z groups;
X is selected from a bond, —NER¹⁵(SO₂)—, —(SO₂)NER¹⁵—, —(SO₂)—, —NER¹⁵C(O)—, —C(O)NER¹⁵—, —NER¹⁵C(O)NER¹⁷—, —NER¹⁵C(O)O—, —NER¹⁵—, C(O)O, OC(O), C(O), O and S;
ER¹² is selected from C₁-C₈ alkylene, C₁-C₈ alkenylene, —C₃-C₈ cycloalkyl-, —C₁-C₈ alkylene-C₃-C₈ cycloalkyl-, and -aryl-, wherein the alkylene, cycloalkyl and aryl groups are optionally substituted by one or more Z groups;
ER¹³, ER¹⁴, ER¹⁵ and ER¹⁷ are each independently selected from H and C₁-C₆ alkyl;
ER¹⁶ is selected from C₁-C₈ alkyl, aryl and a 3- to 14-membered heterocyclic group, the heterocyclic group including one or more heteroatoms selected from N, O and S;
Z is independently selected from OH, aryl, O-aryl, C₇-C₁₄ aralkyl, O—C₇-C₁₄ aralkyl, Cᵣ C₆ alkyl, C₁-C₆ alkoxy, NER¹⁹(SO₂)ER²¹, (SO₂)NER¹⁹ER²¹, (SO₂)ER²⁰, NER¹⁹C(O)ER²⁰, C(O)NER¹⁹ER²⁰, NER¹⁹C(O)NER²⁰ER¹⁸, NER¹⁹C(O)OER²⁰, NER¹⁹ER²¹, C(O)OER¹⁹, C(O)ER¹⁹, SER¹⁹, OER¹⁹, oxo, CN, NO₂, and halogen, wherein the alkyl, alkoxy, aralkyl and aryl groups are each optionally substituted by one or more substituents selected from OH, halogen, C₁-C₄ haloalkyl and C₁-C₄ alkoxy;
ER¹⁸ and ER²⁰ are each independently selected from H and C₁-C₆ alkyl;
ER¹⁹ and ER²¹ are each independently selected from H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl and C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or
ER¹⁹ and ER²⁰ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy;
ER²² is selected from H, halogen, C₁-C₈ alkyl, C₁-C₈ alkoxy, aryl, O-aryl, S(O)₂-aryl, S(O)₂—C₁-C₆ alkyl, S(O)₂NER²³ER²⁴, NHS(O)₂NER²³ER²⁴, a C₃-C₁₄ carbocyclic group, a 3- to 14-membered heterocyclic group, the heterocyclic group including one or more heteroatoms selected from N, O and S, and O-(3- to 14-membered heterocyclic group, the heterocyclic group including one or more heteroatoms selected from N, O and S), wherein the alkyl, aryl, carbocyclic and heterocyclic groups are each optionally substituted by one or more Z groups;
ER²³ and ER²⁴ are each independently selected from H, C₁-C₈ alkyl and C₃-C₈ cycloalkyl; or
ER²³ and ER²⁴ together with the nitrogen atom to which they are attached form a 5- to 10-membered heterocyclic group, optionally including one or more further heteroatoms selected from N, O and S, wherein the heterocyclic group is optionally substituted by one or more Z groups;
n is 0, 1 or 2;
and p are each independently an integer from 0 to 6; and
q is 0, 1, 2 or 3;
with the proviso that when n is 0, at least one of ER⁶, ER⁷, ER⁸, ER⁹, ER¹⁰ and ER¹¹ is other than H.

3. The composition of claim 1, wherein the ENaC inhibitor is amiloride.

4. The composition of claim 1, wherein the ABC transporter modulator of Formula A comprises a compound of Formula A1, 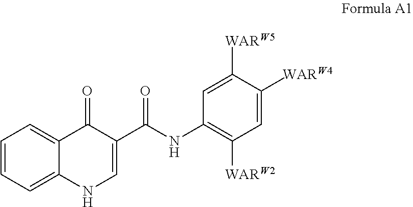
or a pharmaceutically acceptable salt thereof, wherein:
Each of WAR^{W2} and WAR^{W4} is independently selected from CN, CF₃, halo, C₂₋₆ straight or branched alkyl, C₃₋₁₂ membered cycloaliphatic, phenyl, a 5-10 membered heteroaryl or 3-7 membered heterocyclic, wherein said heteroaryl or heterocyclic has up to 3 heteroatoms selected from O, S, or N, wherein said WAR^{W2} and WAR^{W4} is independently and optionally substituted with up to three substituents selected from —OAR′, —CF₃, —OCF₃, SAR′, S(O)AR′, SO₂AR′, —SCF₃, halo, CN, —COOAR′, —COAR′, —O(CH₂)₂N(AR′)₂, —O(CH₂)N(AR′)₂, —CON(AR′)₂, —(CH₂)₂OAR′, —(CH₂)OAR′, —CH₂CN, optionally substituted phenyl or phenoxy, —N(AR′)₂, —NR′C(O)OAR′, —NR′C(O)AR′, —(CH₂)₂N(AR′)₂, or —(CH₂)N(AR′)₂;
WAR^{W5} is selected from hydrogen, —OCF₃, —CF₃, —OH, —OCH₃, —NH₂, —CN, —CHF₂, —NHR′, —N(AR′)₂, —NHC(O)AR′, —NHC(O)OAR′, —NHSO₂AR′, —CH₂OH, —CH₂N(AR′)₂, —C(O)OAR′, —SO₂NHAR′, —SO₂N(AR′)₂, or —CH₂NHC(O)OAR′; and
Each AR′ is independently selected from an optionally substituted group selected from a C₁₋₈ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or two occurrences of R′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur;
provided that:
WAR^{W2} and WAR^{W4} are not both —Cl; and
WAR^{W2}, WAR^{W4} and WAR^{W5} are not —OCH₂CH₂Ph, —OCH₂CH₂(2-trifluoromethyl-phenyl), —OCH₂CH₂-(6,7-dimethoxy-1,2,3,4-tetrahydroisoquinolin-2-yl), or substituted 1H-pyrazol-3-yl.

5. The composition of claim 4, wherein in the compound of Formula A1, each of WAR^{W2} and WAR^{W4} is independently selected from CN, CF₃, halo, C₂₋₆ straight or branched alkyl, C₃₋₁₂ membered cycloaliphatic, or phenyl, wherein said WAR^{W2} and WAR^{W4} is independently and optionally substituted with up to three substituents selected from —OR′, —CF₃, —OCF₃, —SCF₃, halo, —COOAR′, —COAR′, —O(CH₂)₂N(AR′)₂, —O(CH₂)N(AR′)₂, —CON(AR′)₂, —(CH₂)₂OAR′, —(CH₂)OAR′, optionally substituted phenyl, —N(AR′)₂, —NC(O)OAR′, —NC(O)AR′, —(CH₂)₂N(AR′)₂, or —(CH₂)N(AR′)₂; and WAR^{W5} is selected from hydrogen, —OCF₃, —CF₃, —OH, —OCH₃, —NH₂, —CN, —NHAR′, —N(AR′)₂, —NHC(O)AR′, —NHC(O)OAR′, —NHSO₂AR′, —CH₂OH, —C(O)OAR′, —SO₂NHAR′, or —CH₂NHC(O)O-(AR′).

6. The composition of claim 4, wherein in the compound of Formula A1 each of WAR^{W2} and WAR^{W4} is independently selected from —CN, —CF₃, C₂₋₆ straight or branched alkyl, C₃₋₁₂ membered cycloaliphatic, or phenyl, wherein each of said WAR^{W2} and WAR^{W4} is independently and optionally substituted with up to three substituents selected from —OAR′, —CF₃, —OCF₃, —SCF₃, halo, —COOAR′, —COAR′, —O(CH₂)₂N(AR′)₂, —O(CH₂)N(AR′)₂, —CON(AR′)₂, —(CH₂)₂OAR′, —(CH₂)OAR′, optionally substituted phenyl, —N(AR′)₂, —NC(O)OAR′, —NC(O)AR′, —(CH₂)₂N(AR′)₂, or —(CH₂)N(AR′)₂; and WAR^{W5} is selected from —OH, —CN, —NHAR′, —N(AR′)₂, —NHC(O)AR′, —NHC(O)OAR′, —NHSO₂AR′, —CH₂OH, —C(O)OAR′, —SO₂NHAR′, or —CH₂NHC(O)O-(AR′).

7. The composition of claim 4, wherein in the compound of Formula A1, WAR^{W2} is a phenyl ring optionally substituted with up to three substituents selected from —OAR′, —CF₃, —OCF₃, —SAR′, —S(O)AR′, —SO₂AR′, —SCF₃, halo, —CN, —COOAR′, —COAR′, —O(CH₂)₂N(AR′)₂, —O(CH₂)N(AR′)₂, —CON(AR′)₂, —(CH₂)₂OAR′, —(CH₂)OAR′, —CH₂CN, optionally substituted phenyl or phenoxy, —N(AR′)₂, —NR′C(O)OAR′, —NR′C(O)AR′, —(CH₂)₂N(AR′)₂, or —(CH₂)N(R′)₂; WAR^{W4} is C₂₋₆ straight or branched alkyl; and WAR^{W5} is —OH.

8. The composition of claim 4, wherein in the compound of Formula A1 each of WAR^{W2} and WAR^{W4} is independently —CF₃, —CN, or a C₂₋₆ straight or branched alkyl.

9. The composition of claim 4, wherein in the compound of Formula A1 each of WAR^{W2} and WAR^{W4} is C₂₋₆ straight or branched alkyl optionally substituted with up to three substituents independently selected from —OR′, —CF₃, —OCF₃, —SAR′, —S(O)AR′, —SO₂AR′, —SCF₃, halo, —CN, —COOAR′, —COAR′, —O(CH₂)₂N(AR′)₂, —O(CH₂)N(AR′)₂, —CON(AR′)₂, —(CH₂)₂OAR′, —(CH₂)OAR′, —CH₂CN, optionally substituted phenyl or phenoxy, —N(AR′)₂, —NR′C(O)OAR′, —NR′C(O)AR′, —(CH₂)₂N(AR′)₂, or —(CH₂)N(AR′)₂.

10. The composition of claim 4, wherein in the compound of Formula A1 each of WAR^{W2} and WAR^{W4} is independently selected from optionally substituted n-propyl, isopropyl, n-butyl, sec-butyl, t-butyl, 1,1-dimethyl-2-hydroxyethyl, 1,1-dimethyl-2-(ethoxycarbonyl)-ethyl, 1,1-dimethyl-3-(t-butoxycarbonyl-amino)propyl, or n-pentyl.

11. The composition of claim 4, wherein in the compound of Formula A1, WAR^{W5} is selected from —CN, —NHAR′, —N(AR′)₂, —CH₂N(AR′)₂, —NHC(O)AR′, —NHC(O)OAR′, —OH, C(O)OAR′, or —SO₂NHAR′.

12. The composition of claim 4, wherein in the compound of Formula A1, WAR^{W5} is selected from —CN, —NH(C₁₋₆ alkyl), —N(C₁₋₆ alkyl)₂, —NHC(O)(C₁₋₆ alkyl), —CH₂NHC(O)O(C₁₋₆ alkyl), —NHC(O)O(C₁₋₆ alkyl), —OH, —O(C₁₋₆ alkyl), —C(O)O(C₁₋₆ alkyl), —CH₂O(C₁₋₆ alkyl), or —SO₂NH₂.

13. The composition of claim 4, wherein in the compound of Formula A1 WAR^{W5} is selected from —OH, —CH₂OH, —NHC(O)OMe, —NHC(O)OEt, —CN, —CH₂NHC(O)O(t-butyl), —C(O)OMe, or —SO₂NH₂.

14. The composition of claim 4, wherein in the compound of Formula A1,
a. WAR^{W2} is C₂₋₆ straight or branched alkyl;
b. WAR^{W4} is C₂₋₆ straight or branched alkyl or monocyclic or bicyclic aliphatic; and
c. WAR^{W5} is selected from —CN, —NH(C₁₋₆ alkyl), —N(C₁₋₆ alkyl)₂, —NHC(O)(C₁₋₆ alkyl), —NHC(O)O(C₁₋₆ alkyl), —CH₂C(O)O(C₁₋₆ alkyl), —OH, —O(C₁₋₆ alkyl), —C(O)O(C₁₋₆ alkyl), or —SO₂NH₂.

15. The composition of claim 4, wherein in the compound of Formula A1,
a. WAR^{W2} is C₂₋₆ alkyl, —CF₃, —CN, or phenyl optionally substituted with up to 3 substituents selected from C₁₋₄ alkyl, —O(C₁₋₄ alkyl), or halo;
b. WAR^{W4} is —CF₃, C₂₋₆ alkyl, or C₆₋₁₀ cycloaliphatic; and
c. WAR^{W5} is —OH, —NH(C₁₋₆ alkyl), or —N(C₁₋₆ alkyl)₂.

16. The composition of claim 4, wherein in the compound of Formula A1, WAR^{W2} is tert-butyl.

17. The composition of claim 4, wherein in the compound of Formula A1, WAR^{W4} is tert-butyl.

18. The composition of claim 4, wherein in the compound of Formula A1, WAR^{W5} is —OH.

19. The composition of claim 4, wherein the compound of Formula A1, comprises Compound 1

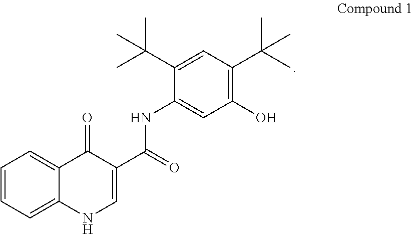 [SMILES: CC(C)(C)c1cc(C(C)(C)C)c(NC(=O)c2c[nH]c3ccccc3c2=O)cc1O]

20. The composition of claim 1, wherein the ABC transporter modulator of Formula C comprises a compound of Formula C1, 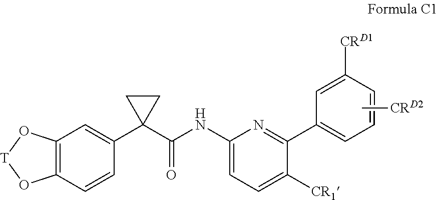
or a pharmaceutically acceptable salt thereof, wherein:
T is —CH₂—, —CH₂CH₂—, —CF₂—, —C(CH₃)₂—, or —C(O)—;
CR₁′ is H, C₁₋₆ aliphatic, halo, CF₃, CHF₂, O(C₁₋₆ aliphatic); and
CR^{D1} or CR^{D2} is Z^{D}CR₉
wherein:
Z^{D} is a bond, CONH, SO₂NH, SO₂N(C₁₋₆ alkyl), CH₂NHSO₂, CH₂N(CH₃)SO₂, CH₂NHCO, COO, SO₂, or CO; and
CR₉ is H, C₁₋₆ aliphatic, or aryl.

21. The composition of claim 20, wherein the compound of Formula C1, comprises Compound 2

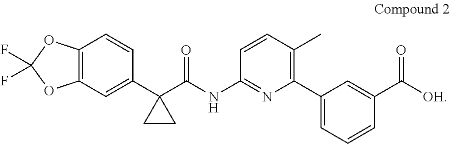 [SMILES: Cc1ccc(NC(=O)C2(c3ccc4c(c3)OC(F)(F)O4)CC2)nc1-c1cccc(C(=O)O)c1]

22. The composition of claim 21, further comprising an ENaC inhibitor of Formula E.

23. The composition of claim 1, wherein the ABC transporter modulator of Formula D comprises a compound of Formula D1, 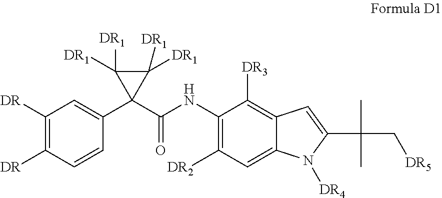
or a pharmaceutically acceptable salt thereof, wherein:
DR is H, OH, OCH₃ or two R taken together form —OCH₂O— or —OCF₂O—;
DR₁ is H or alkyl;
DR₂ is H or F;
DR₃ is H or CN;
DR₄ is H, —CH₂CH(OH)CH₂OH, —CH₂CH₂N⁺(CH₃)₃, or —CH₂CH₂OH; and
DR₅ is H, OH, —CH₂CH(OH)CH₂OH, —CH₂OH, or DR₄ and DR₅ taken together form a five membered ring.

24. The composition of claim 23, wherein the compound of Formula D1 comprises Compound 3

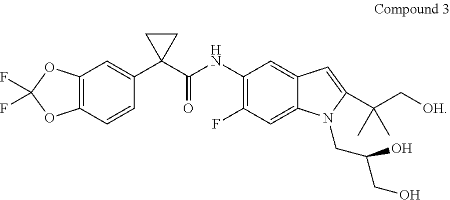 [SMILES: CC(C)(CO)c1cc2cc(NC(=O)C3(c4ccc5c(c4)OC(F)(F)O5)CC3)c(F)cc2n1C[C@@H](O)CO]

25-42. (canceled)

43. A method of treating a CFTR mediated disease in a human comprising administering to the human an effective amount of a pharmaceutical composition according to claim 1, wherein the CFTR mediated disease is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulinemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurohypophyseal DI, nephrogenic DI, Charcot-Marie Tooth syndrome, Pelizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington's, spinocerebellar ataxia type I, spinal and bulbar muscular atrophy, dentatorubral pallidoluysian atrophy, and myotonic dystrophy, as well as spongiform encephalopathies, such as hereditary Creutzfeldt-Jakob disease (due to prion protein processing defect), Fabry disease, Gerstmann-Sträussler-Scheinker syndrome, COPD, dry-eye disease, or Sjogren's disease, Osteoporosis, Osteopenia, bone healing and bone growth (including bone repair, bone regeneration, reducing bone resorption and increasing bone deposition), Gorham's Syndrome, chloride channelopathies such as myotonia congenita (Thomson and Becker forms), Bartter's syndrome type III, Dent's disease, epilepsy, hyperekplexia, lysosomal storage disease, Angelman syndrome, and Primary Ciliary Dyskinesia (PCD), a term for inherited disorders of the structure and/or function of cilia, including PCD with situs inversus (also known as Kartagener syndrome), PCD without situs inversus and ciliary aplasia.

44-57. (canceled)
